(19) United States
(12) Patent Application Publication
(10) Pub. No.: US 2025/0243210 A1
(43) Pub. Date: Jul. 31, 2025

(54) CRBN E3 LIGASE LIGAND COMPOUND, PROTEIN DEGRADER DEVELOPED BASED THEREON AND THEIR APPLICATIONS

(71) Applicant: GLUETACS THERAPEUTICS (SHANGHAI) CO., LTD., Shanghai (CN)

(72) Inventors: Xiaobao YANG, Shanghai (CN); Renhong SUN, Shanghai (CN); Baoyin ZHAO, Shanghai (CN)

(21) Appl. No.: 18/703,316

(22) PCT Filed: Oct. 20, 2022
(86) PCT No.: PCT/CN2022/126516

§ 371 Date: Apr. 19, 2024

(30) Foreign Application Priority Data
Oct. 22, 2021 (CN) 202111231963.9

(51) Int. Cl. C07D 487/08; A61K 31/4545; A61K 31/496; A61K 31/4995; A61K 47/55; A61P 35/00; C07D 401/04; C07D 401/14; C07D 471/08

(52) U.S. Cl. CPC C07D 487/08; A61K 31/4545; A61K 31/496; A61K 31/4995; A61K 47/55; A61P 35/00; C07D 401/04; C07D 401/14; C07D 471/08

(57) ABSTRACT

The present disclosure provides compounds of Formula (I), (II) and (III) or salts, enantiomers, stereoisomers, solvates, isotopically enriched analogs, prodrugs, or polymorphs thereof, and uses thereof. The present disclosure also provides pharmaceutical compositions comprising, as an active ingredient, the compounds of Formula (I), (II) and (III) or salts, enantiomers, stereoisomers, solvates, isotopically enriched analogs, prodrugs, or polymorphs thereof, and uses thereof.

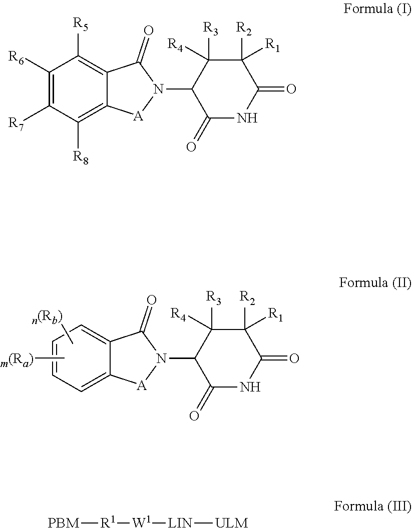

This application is the U.S. national phase of International Application No. PCT/CN2022/126516 filed Oct. 20, 2022 which designated the U.S. and claims priority to CN Patent Application No. 202111231963.9 filed Oct. 22, 2021, the entire contents of each of which are hereby incorporated by reference.

### TECHNICAL FIELD

The present disclosure relates to cereblon (CRBN) E3 ubiquitin ligase ligand compounds of Formula (I) and Formula (II), protein degrader compounds of Formula (III) comprising the same, and their applications. The CRBN E3 ubiquitin ligase ligand compounds can effectively treat or prevent cereblon protein mediated diseases or disorders. The protein degrader compounds of Formula (III) can effectively treat or prevent diseases or disorders associated with the proteins degraded.

### BACKGROUND

Bifunctional proteolysis-targeting drugs consist of three moieties, one end of which is a ligand warhead that can bind to a specific target protein, and the other end is a small molecule ligand of E3 ubiquitin ligase, both of which are connected together through linkers of different lengths and categories located in the middle. The bifunctional proteolysis-targeting small molecules can utilize the binding effects of the ligands at both ends to simultaneously bind to a specific target protein and E3 ubiquitin ligase, thereby recruiting E3 ubiquitin ligase to the vicinity of the specific target protein and enabling E3 ubiquitin ligase to ubiquitinate the target protein. The polyubiquitinated target protein will be recognized and degraded by proteasomes in the body. The biggest difference between the bifunctional proteolysis-targeting drugs and traditional small molecule-based inhibitor drugs is that the former mobilize the entire cell as a drug effector unit. This drug action mode only requires small molecule drugs to briefly bind to the target protein and tag it for degradation. Therefore, a low drug dose can meet the requirements, greatly reducing the risk of off-target effects, and potentially eliminating tumor progression caused by abnormal expression of driver genes and drug resistance caused by acquired mutations in driver genes.

The E3 ubiquitin ligases involved in the design of proteolysis-targeting drugs include over 500 different proteins, each with a distinct cellular expression profile, and can be classified into multiple categories based on the structural elements related to their E3 functional activity. Currently, only a few ligases have been successfully applied in preclinical degraders research. Among them, Cereblon (CRBN) E3 ubiquitin ligase is one of the most widely used E3 ubiquitin ligases. The known ligands for CRBN-type E3 ubiquitin ligase (CRBN ligands) include thalidomide and its analogs pomalidomide and lenalidomide, all of which possess a phthalimide backbone. CRBN-type E3 ubiquitin ligase ligands can also act as molecular glues to induce the degradation of specific proteins. Cereblon (CRBN) forms a functional E3 ubiquitin ligase complex with damaged DNA binding protein 1 (DDB1) and Cullin 4A, where CRBN serves as the substrate receptor. Molecular glue degraders like thalidomide bind to CRBN, inducing it to recognize new substrate proteins. The binding leads to the ubiquitination of these proteins, followed by their recognition and degradation by proteasomes.

CRBN ligands have been widely used in protein degradation, and a series of small molecules protein degraders based on CRBN ubiquitin ligases, which utilize the function of the CRBN E3 ubiquitin ligase complex, have gradually been developed. Since CRBN ligands can serve not only as molecular glue degraders to induce the degradation of different substrate proteins but also as a component of bifunctional proteolysis-targeting drugs to achieve effective degradation of specific pathogenic proteins, and given the unavoidable side effects and drug resistance issues associated with various existing domides compounds, such as thalidomide, it is of great significance to design novel and optimized E3 ubiquitin ligase ligands and study their binding ability to CRBN and evaluate their biological activity. Furthermore, these E3 ubiquitin ligase ligands can potentially be applied in bifunctional proteolysis-targeting small molecules.

Therefore, there is an urgent need for a series of novel CRBN ligands that can serve as effective molecular glue degraders and can also be further used to synthesize corresponding bifunctional proteolysis-targeting degraders for the treatment and/or prevention of diseases or conditions mediated or associated with the proteins degraded.

### SUMMARY OF INVENTION

In view of the above, the objectives of the present disclosure are to provide novel CRBN ligands, protein degrader molecules designed based on the CRBN ligands and various target protein ligands, as well as their applications and usage methods. The advantages of the compounds of the present disclosure lie in their ability to exhibit a wide range of pharmacological activities through the degradation/inhibition of diverse types or families of pathogenic proteins.

To achieve the above objectives and other related goals, in one aspect, the present disclosure provides novel CRBN ligands that can themselves act as molecular glues binding to CRBN E3 ligase, and subsequently induce the degradation of CRBN substrate proteins, including but not limited to GSPT1, IKZF1, IKZF2, IKZF3 (Aiolos), and IKZF4 proteins.

In some embodiments, the novel CRBN ligands of the present disclosure can be compounds of Formula (I):

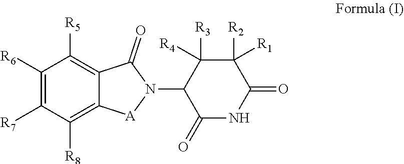

or salts, stereoisomers (including enantiomers), solvates, isotopically enriched analogs, prodrugs, or polymorphs thereof
wherein A represents CO, CH₂ or CD₂;
R₁, R₂, R₃ and R₄ are the same or different and each independently represent e.g., hydrogen, deuterium, halogen, optionally deuterated C₁₋₆ alkyl, optionally deuterated C₁₋₆ alkoxy, or halogenated C₁₋₆ alkyl;
R₅, R₆, R₇ and R₈ are the same or different and each independently represent e.g., R₉, hydrogen, deuterium, halogen, hydroxy, mercapto, nitro, amino, cyano, optionally deuterated C₁₋₆ alkyl, optionally deuterated C₁₋₆ alkoxy, halogenated C₁₋₆ alkyl, C₂₋₆ alkenyl, or C₂₋₆ alkynyl, wherein one to three of R₅, R₆, R₇ and R₈ represent R₉;
R₉ represents R—U— group, wherein
   U represents a bond, or U represents N(R₁₀) or O, wherein R₁₀ represents e.g., H or optionally deuterated C₁₋₆ alkyl;
   R represents optionally substituted nitrogen-containing heterocyclyl, said optionally substituted nitrogen-containing heterocyclyl is optionally substituted nitrogen-containing monocyclic heterocyclyl or optionally substituted nitrogen-containing bridged heterocyclyl;
      wherein
when two to four of R₅, R₆, R₇, and R₈ independently represent R₉, the compound of Formula (I) is optionally partially or fully deuterated; or
when only one of R₅, R₆, R₇, and R₈ represents R₉, the compound of Formula (I) is optionally partially or fully deuterated;
wherein in case that only one of R₅, R₆, R₇, and R₈ represents R₉ and the compound of Formula (I) is not deuterated:
   when R represents optionally substituted nitrogen-containing bridged heterocyclyl, the following compounds are excluded:
5-(2,5-diazabicyclo[2.2.1]heptan-2-yl)-2-(2,6-dioxopyridin-3-yl)isoindoline-1,3-dione;
5-((1R,4R)2,5-diazabicyclo[2.2.1]heptan-2-yl)-2-(2,6-dioxopyridin-3-yl)-6-fluoroisoindoline-1,3-dione;
5-(2,5-diazabicyclo[2.2.1]heptan-2-yl)-2-(2,6-dioxopyridin-3-yl)-6-fluoroisoindoline-1,3-dione;
3-(5-(2,5-diazabicyclo[2.2.1]heptan-2-yl)-1-oxoisoindolin-2-yl)piperidine-2,6-dione;
3-(5-(8-azabicyclo[3.2.1]octan-3-yl)-1-oxoisoindolin-2-yl)piperidine-2,6-dione; and
3-(5-(9-ethyl-3-methyl-3,9-diazabicyclo[3.3.1]nonan-7-yl)-1-oxoisoindolin-2-yl)piperidine-2,6-dione; or
   when A represents CO and one of R₆ and R₇ represents R₉, where R represents substituted nitrogen-containing monocyclic heterocyclyl, and the other one of R₆ and R₇, as well as R₅ and R₈ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, one to three of the other one of R₆ and R₇ as well as R₅ and R₈ represents halogen, and the nitrogen-containing monocyclic heterocyclyl is substituted with one or more substituents selected from the group consisting of hydroxy, amino, mercapto, nitro, halogen, cyano, C₁₋₆ alkyl, C₁₋₆ alkoxy, halogenated C₁₋₆ alkyl, NH₂—C₁₋₆ alkylene, C₁₋₆ alkyl-NH—, C₁₋₆ alkyl-NHC(O)—, C₁₋₆ alkyl-C(O)NH— or any combination thereof;
   when A represents CO and one of R₆ and R₇ represents R₉, where R represents unsubstituted nitrogen-containing monocyclic heterocyclyl, the other one of R₆ and R₇ represents hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, and R₅ and R₈ each independently represent halogen;
   when A represents CO and one of R₅ and R₈ represents R₉, where R represents substituted nitrogen-containing monocyclic heterocyclyl, and the other one of R₅ and R₈, as well as R₆ and R₇ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, one to three of the other one of R₅ and R₈ as well as R₆ and R₇ represent halogen, and the nitrogen-containing monocyclic heterocyclyl is substituted with one or more substituents selected from the group consisting of hydroxy, amino, mercapto, nitro, halogen, cyano, CHO, C₁₋₆ alkyl, halogenated C₁₋₆ alkyl, C₃₋₆ cycloalkyl, C₁₋₆ alkoxy, C₁₋₆ alkyl-NH—, NH₂—C₁₋₆ alkylene, C₁₋₆ alkyl-NHC(O)—, C₁₋₆ alkyl-C(O)NH— or any combination thereof;
   when A represents CO and one of R₅ and R₈ represents R₉, where R represents unsubstituted nitrogen-containing monocyclic heterocyclyl, the other one of R₅ and R₈ and/or R₇ each independently represent halogen, and R₆ represents hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl;
   when A represents CH₂ and one of R₆ and R₇ represents R₉, where R represents substituted nitrogen-containing monocyclic heterocyclyl, and the other one of R₆ and R₇, as well as R₅ and R₈ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, one to three of the other one of R₆ and R₇ as well as R₅ and R₈ represents halogen, and the nitrogen-containing monocyclic heterocyclyl is substituted with one or more substituents selected from the group consisting of hydroxy, amino, mercapto, nitro, halogen, CHO, cyano, C₁₋₆ alkyl, halogenated C₁₋₆ alkyl, C₃₋₆ cycloalkyl, C₁₋₆ alkoxy, C₁₋₆ alkyl-NH—, NH₂—C₁₋₆ alkylene, C₁₋₆ alkyl-NHC(O)—, C₁₋₆ alkyl-C(O)NH— or any combination thereof;
   when A represents CH₂ and one of R₆ and R₇ represents R₉, where R represents unsubstituted nitrogen-containing monocyclic heterocyclyl, R₅ represents halogen, and the other one of R₆ and R₇ and/or R₈ each independently represent halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl;
   when A represents CH₂ and one of R₅ and R₈ represents R₉, where R represents substituted nitrogen-containing monocyclic heterocyclyl, and the other one of R₅ and R₈, as well as R₆ and R₇ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, one to three of the other one of R₅ and R₈ as well as R₆ and R₇ represent halogen, and the nitrogen-containing monocyclic heterocyclyl is substituted with one or more substituents selected from the group consisting of hydroxy, amino, mercapto, nitro, halogen, CHO, cyano, C₁₋₆ alkyl, halogenated C₁₋₆ alkyl, C₃₋₆ cycloalkyl, C₁₋₆ alkoxy, C₁₋₆ alkyl-NH—, NH₂—C₁₋₆ alkylene, C₁₋₆ alkyl-NHC(O)—, C₁₋₆ alkyl-C(O)NH— or any combination thereof; or
   when A represents CH₂ and one of R₅ and R₈ represents R₉, where R represents unsubstituted nitrogen-containing monocyclic heterocyclyl, and the other one of R₅ and R₈, as well as R₆ and R₇ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, the other one of R₅ and R₈ as well as R₆ and R₇ are not simultaneously hydrogen, and when R₆ is halogen, the other one of R₅ and R₈, as well as R₇ are not simultaneously hydrogen.

In another aspect, the CRBN ligands of the present disclosure can be compounds of Formula (II):

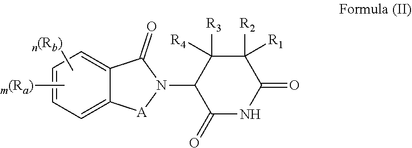

or salts, stereoisomers (including enantiomers), solvates, isotopically enriched analogs, prodrugs, or polymorphs thereof
wherein A represents CO, CH₂ or CD₂;
R₁, R₂, R₃ and R₄ are the same or different and each independently represent e.g., hydrogen, deuterium, halogen, optionally deuterated C₁₋₆ alkyl, optionally deuterated C₁₋₆ alkoxy, or halogenated C₁₋₆ alkyl;
(R_{b})ₙ indicates that benzene ring is substituted with n R_{b} substituents, and each R_{b} are the same or different and each independently represent e.g., hydrogen, deuterium, halogen, hydroxy, mercapto, nitro, amino, cyano, optionally deuterated C₁₋₆ alkyl, optionally deuterated C₁₋₆ alkoxy, halogenated C₁₋₆ alkyl, C₂₋₆ alkenyl or C₂₋₆ alkynyl;
(Rₐ)ₘ indicates that the benzene ring is substituted with m Rₐ substituents, and each Rₐ are the same or different and each independently represents the structure of the following formula:

W-LIN—U₂—R_{c}—U₁—,
wherein
   U₁ and U₂ are the same or different and each independently represent e.g., a bond, N(R_{d}) or O, wherein R_{d} represents e.g., H or optionally deuterated C₁₋₆ alkyl;
   R_{c} represents optionally substituted nitrogen-containing monocyclic heterocyclylene or optionally substituted nitrogen-containing bridged heterocyclylene;
   W represents hydrogen, optionally substituted nitrogen-containing monocyclic heterocyclyl, optionally substituted nitrogen-containing bridged heterocyclyl, optionally substituted cycloalkyl, optionally substituted heteroaryl, optionally substituted aryl, C₂₋₆ alkenyl, C₂₋₆ alkynyl, a leaving group, CHO, COOH, NH₂, hydroxy or mercapto; and
   wherein in case that the compound of Formula (II) is partially or fully deuterated:
      when W represents optionally substituted nitrogen-containing monocyclic heterocyclyl, optionally substituted nitrogen-containing bridged heterocyclyl, optionally substituted cycloalkyl, optionally substituted heteroaryl or optionally substituted aryl, LIN represents a bond, optionally substituted methylene, or optionally substituted linear or branched C₂₋₃₀ alkylene, wherein one or more groups Rₑ and/or one or more groups R_{f} and/or any combination of one or more groups Rₑ and R_{f} are optionally inserted into the backbone carbon chain of the linear or branched C₂₋₃₀ alkylene group, wherein carbon-carbon bond between one or more pairs of adjacent carbon atoms in the backbone carbon chain is interrupted by the group Rₑ, R_{f}, or a combination of Rₑ and R_{f}; wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl, and in case that two or more groups Rₑ are inserted into the backbone carbon chain of the linear or branched C₂₋₃₀ alkylene group, the two or more groups Rₑ are not directly connected to each other; and wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of e.g., deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano or any combination thereof; or
      when W represents hydrogen, C₂₋₆ alkenyl, C₂₋₆ alkynyl, a leaving group, CHO, COOH, NH₂, hydroxy or mercapto, LIN represents optionally substituted methylene, or optionally substituted linear or branched C₂₋₃₀ alkylene, wherein one or more groups Rₑ and/or one or more groups R_{f} and/or any combination of one or more groups Rₑ and R_{f} are optionally inserted into the backbone carbon chain of the linear or branched C₂₋₃₀ alkylene group, wherein carbon-carbon bond between one or more pairs of adjacent carbon atoms in the backbone carbon chain is interrupted by the group Rₑ, R_{f}, or a combination of Rₑ and R_{f}; wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl, and in case that two or more groups Rₑ are inserted into the backbone carbon chain of the linear or branched C₂₋₃₀ alkylene group, the two or more groups Rₑ are not directly connected to each other; and wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of e.g., deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano or any combination thereof; or
   in case that the compound of Formula (II) is not deuterated, LIN represents optionally substituted methylene, and W represents optionally substituted nitrogen-containing monocyclic heterocyclyl or optionally substituted nitrogen-containing bridged heterocyclyl; and
m represents an integer of 1, 2, 3 or 4, n represents an integer of 0, 1, 2 or 3, and the sum of m and n is an integer of 1, 2, 3 or 4;
   with the proviso that when the compound of Formula (II) is not deuterated, the following compounds are excluded:
3-(4,6-difluoro-1-oxo-5-(4-(piperidin-4-ylmethyl)piperazin-1-yl)isoindolin-2-yl)piperidine-2,6-dione;
2-(2,6-dioxopiperidin-3-yl)-5-(4-(piperidin-4-ylmethyl)piperazin-1-yl)isoindoline-1,3-dione;
3-(4-chloro-1-oxo-5-(4-(piperidin-4-ylmethyl)piperidin-1-yl)isoindolin-2-yl)piperidine-2,6-dione;
5-(4-(azetidin-3-ylmethyl)piperazin-1-yl)-2-(2,6-dioxopiperidin-3-yl)isoindoline-1,3-dione; and
2-(2,6-dioxopiperidin-3-yl)-5-(3-(piperazin-1-ylmethyl)azetidin-1-yl)isoindoline-1,3-dione.

In another aspect, the protein degrader molecules of the present disclosure are compounds of Formula (III):

PBM-R¹—W¹-LIN—ULM Formula (III)

or salts, stereoisomers (including enantiomers), solvates, isotopically enriched analogs, prodrugs, or polymorphs thereof,
wherein PBM-R¹—W¹— represents a targeting moiety capable of binding protein, ULM represents a E3 ubiquitin ligase ligand moiety, LIN is a linker moiety, and PBM-R¹—W¹— is covalently bonded to ULM through LIN;
ULM represents the structure of Formula (ULM):

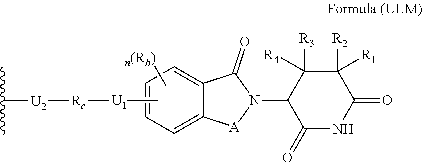

wherein A represents CO, CH₂ or CD₂;
R₁, R₂, R₃ and R₄ are the same or different and each independently represent e.g., hydrogen, deuterium, halogen, optionally deuterated C₁₋₆ alkyl, optionally deuterated C₁₋₆ alkoxy, or halogenated C₁₋₆ alkyl;
(R_{b})ₙ indicates that benzene ring is optionally substituted with n R_{b} groups, and each R_{b} are the same or different and each independently represent e.g., hydrogen, deuterium, halogen, hydroxy, mercapto, nitro, amino, cyano, optionally deuterated C₁₋₆ alkyl, optionally deuterated C₁₋₆ alkoxy, halogenated C₁₋₆ alkyl, C₂₋₆ alkenyl or C₂₋₆ alkynyl;
n represents an integer of 0, 1, 2 or 3;
U₁ and U₂ are the same or different and each independently represent e.g., a bond, N(R_{d}) or O, wherein R_{d} represents e.g., H or optionally deuterated C₁₋₆ alkyl;
R_{c} represents optionally substituted nitrogen-containing monocyclic heterocyclylene or optionally substituted nitrogen-containing bridged heterocyclylene;
LIN represents a bond, optionally substituted methylene, or optionally substituted linear or branched C₂₋₃₀ alkylene, wherein one or more groups Rₑ and/or one or more groups R_{f} and/or any combination of one or more groups Rₑ and R_{f} are optionally inserted into the backbone carbon chain of the linear or branched C₂₋₃₀ alkylene group, wherein carbon-carbon bond between one or more pairs of adjacent carbon atoms in the backbone carbon chain is interrupted by the group Rₑ, R_{f}, or a combination of Rₑ and R_{f}; wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl, and in case that two or more groups Rₑ are inserted into the backbone carbon chain of the linear or branched C₂₋₃₀ alkylene group, the two or more groups Rₑ are not directly connected to each other; and wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of e.g., deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano or any combination thereof; W¹ is t interconnected rings W² and represented by the following formula:

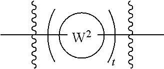

each ring W² are the same or different and each independently represent optionally substituted nitrogen-containing monocyclic heterocyclylene, optionally substituted nitrogen-containing bridged heterocyclylene, or optionally substituted cycloalkylene, and
t represents an integer of 1 or 2; and
R¹ represents:
   a bond, —O—, optionally substituted C₁₋₆ alkylene (e.g., methylene), —N(R^{1a})—R^{2a}—***(e.g.,

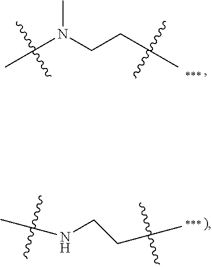

—O—R^{2a}—*** (e.g.,

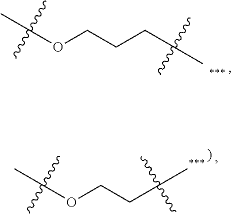 [SMILES: *CCCO* ; *CCO*]

—C(O)NH—R^{2a}—***, or —NHC(O)—R^{2a}—*** (e.g.,

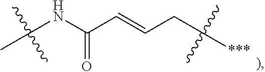

wherein symbol *** indicates the point of attachment to W¹, R^{1a} is H or C₁₋₆ alkyl, and R^{2a} is optionally substituted C₁₋₆ alkylene or optionally substituted C₁₋₆ alkenylene; or
C₆₋₁₀ aryl or heteroaryl containing one or two 5- to 7-membered rings and 1 to 4 heteroatoms selected from nitrogen, oxygen, and sulfur, wherein the C₆₋₁₀ aryl and heteroaryl are each independently optionally substituted with one or more R^{3a}, and each R^{3a} are the same or different and are each independently e.g., halogen, hydroxy, cyano, amino, nitro, C₁₋₆ alkyl, halogenated C₁₋₆ alkyl, C₁₋₆ alkoxy, or halogenated C₁₋₆ alkoxy;
   wherein one or more hydrogens of the compound of Formula (III) are optionally replaced with deuterium;
   wherein when the compound of Formula (III) is not deuterated, the following compounds are excluded:

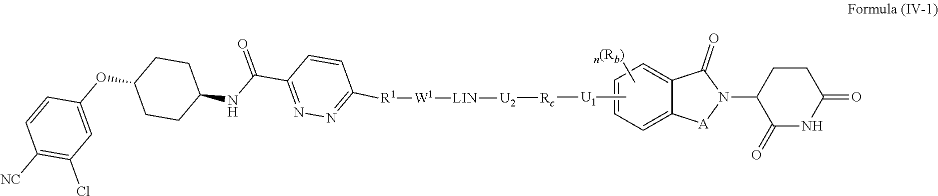

in Formula (IV-1), R¹ represents a bond, W¹ represents piperidinylene, LIN represents methylene, R_{c} represents piperazinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents fluoro, n represents an integer of 1, 2 or 3, and U₁ and U₂ are the same or different and each independently represent a bond, NH or O; or
in Formula (IV-1), R¹ represents a bond, W¹ represents piperazinylene, LIN represents methylene, R_{c} represents piperidinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents fluoro, n represents an integer of 1, 2 or 3, and U₁ and U₂ are the same or different and each independently represent a bond, NH or O;

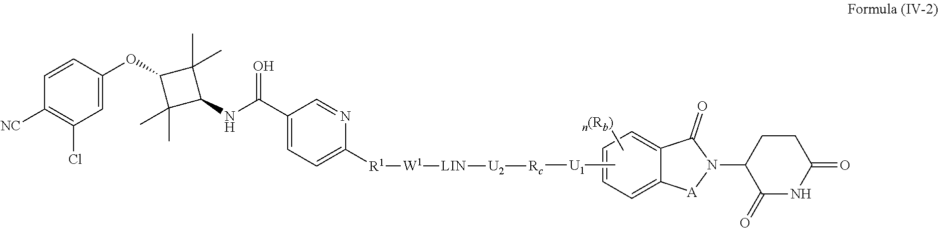

in Formula (IV-2), R¹ represents a bond, W¹ represents piperidinylene, LIN represents methylene, R_{c} represents piperazinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents fluoro, n represents an integer of 1, 2 or 3, and U₁ and U₂ are the same or different and each independently represent a bond, NH or O; or
in Formula (IV-2), R¹ represents a bond, W¹ represents piperazinylene, LIN represents methylene, R_{c} represents piperidinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents fluoro, and n represents an integer of 1, 2 or 3, [symbol] U₁ and U₂ are the same or different and each independently represent a bond, NH or O;

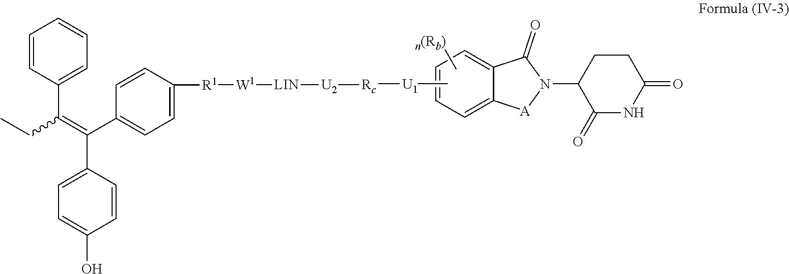

in Formula (IV-3), R¹ represents a bond or —O—CH₂—CH₂—, W¹ represents piperidinylene, LIN represents methylene, R_{c} represents piperazinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents hydrogen, and U₁ and U₂ are the same or different and each independently represent a bond, NH or O; or
in Formula (IV-3), R¹ represents a bond or —O—CH₂—CH₂—, W¹ represents piperazinylene, LIN represents methylene, R_{c} represents piperidinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents hydrogen, and U₁ and U₂ are the same or different and each independently represent a bond, NH or O;
and

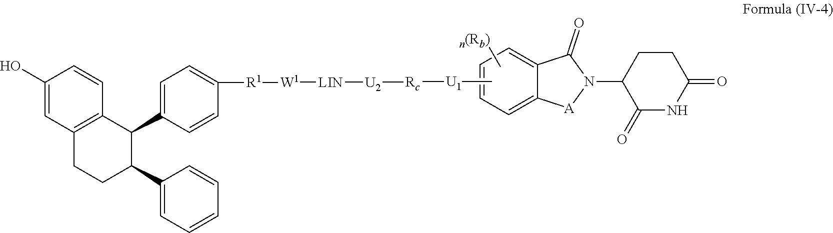

in Formula (IV-4), R¹ represents a bond or represents —O—CH₂—CH₂—, W¹ represents piperidinylene, LIN represents methylene, R_{c} represents piperazinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents hydrogen, and U₁ and U₂ are the same or different and each independently represent a bond, NH or O; or
in Formula (IV-4), R¹ represents a bond or represents —O—CH₂—CH₂—, W¹ represents piperazinylene, LIN represents methylene, R_{c} represents piperidinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents hydrogen, and U₁ and U₂ are the same or different and each independently represent a bond, NH or O.

In a further aspect, the present disclosure provides a pharmaceutical composition comprising the compound of Formula (I) or salts, stereoisomers (including enantiomers), solvates, isotopically enriched analogs, prodrugs, or polymorphs thereof, and at least one pharmaceutically acceptable carrier.

In a further aspect, the present disclosure provides a pharmaceutical composition comprising the compound of Formula (II) or salts, stereoisomers (including enantiomers), solvates, isotopically enriched analogs, prodrugs, or polymorphs thereof, and at least one pharmaceutically acceptable carrier.

In a further aspect, the present disclosure provides a pharmaceutical composition comprising the compound of Formula (III) or salts, stereoisomers (including enantiomers), solvates, isotopically enriched analogs, prodrugs, or polymorphs thereof, and at least one pharmaceutically acceptable carrier.

In a further aspect, the present disclosure further provides a medicine kit or reagent kit comprising: the compound of Formula (I) or a pharmaceutically acceptable salt, or the pharmaceutical composition comprising the same; or the compound of Formula (II) or a pharmaceutically acceptable salt, or the pharmaceutical composition comprising the same; or the compound of Formula (III) or a pharmaceutically acceptable salt, or the pharmaceutical composition comprising the same.

In a further aspect, the present disclosure provides the compound of Formula (I) or salts, stereoisomers (including enantiomers), solvates, isotopically enriched analogs, prodrugs, or polymorphs thereof, or the pharmaceutical composition comprising the same as active ingredient for use as a medicament.

In a further aspect, the present disclosure provides the compound of Formula (I) or salts, stereoisomers (including enantiomers), solvates, isotopically enriched analogs, prodrugs, or polymorphs thereof, or the pharmaceutical composition comprising the same as active ingredient for use in the prevention or treatment of cereblon protein-mediated disease or disorder.

In some embodiments, the cereblon protein-mediated disease or disorder includes, but not limited to, tumor, infectious disease, autoinflammatory disease, inflammatory disease, autoimmune disease, neurological disease, respiratory disease, anemia, hemorrhagic shock, transplant rejection, multiple organ dysfunction syndrome (MODS), sarcoidosis, adult respiratory distress syndrome, congestive heart failure, myocardial infarction, Unverricht's syndrome, Richter syndrome (RS), acute liver failure, or diabetes.

In a further aspect, the present disclosure provides the compound of Formula (II) or salts, stereoisomers (including enantiomers), solvates, isotopically enriched analogs, prodrugs, or polymorphs thereof, or the pharmaceutical composition comprising the same as active ingredient for use as a medicament.

In a further aspect, the present disclosure provides the compound of Formula (II) or salts, stereoisomers (including enantiomers), solvates, isotopically enriched analogs, prodrugs, or polymorphs thereof, or the pharmaceutical composition comprising the same as active ingredient for use in the prevention or treatment of cereblon protein-mediated disease or disorder.

In a further aspect, the present disclosure provides the compound of Formula (III) or salts, stereoisomers (including enantiomers), solvates, isotopically enriched analogs, prodrugs, or polymorphs thereof, or the pharmaceutical composition comprising the same as active ingredient for use as a medicament.

In a further aspect, the present disclosure provides the compound of Formula (III) or salts, stereoisomers (including enantiomers), solvates, isotopically enriched analogs, prodrugs, or polymorphs thereof, or the pharmaceutical composition comprising the same as active ingredient for use in the treatment and/or prevention of a disease or disorder selected from the group consisting of tumor, infectious disease, autoinflammatory disease, inflammatory disease, autoimmune disease, neurological disease, respiratory disease, anemia, hemorrhagic shock, transplant rejection, multiple organ dysfunction syndrome (MODS), sarcoidosis, adult respiratory distress syndrome, congestive heart failure, myocardial infarction, Unverricht's syndrome, Richter syndrome (RS), acute liver failure, diabetes, Kennedy disease, seborrheic alopecia, hirsutism, skin disease, cardiovascular disease, dysfunctional uterine bleeding, anemia, pediatric aplastic anemia, endometriosis, transplant rejection, polycystic ovary syndrome, or thyroid disease.

In a further aspect, the present disclosure provides use of the compound of Formula (I) or salts, stereoisomers (including enantiomers), solvates, isotopically enriched analogs, prodrugs, or polymorphs thereof for the preparation of the compound of Formula (III) or salts, enantiomers, stereoisomers, solvates, isotopically enriched analogs, prodrugs, or polymorphs thereof of the present disclosure.

In a further aspect, the present disclosure also provides a method for preparing the compound of Formula (III) or salts, enantiomers, stereoisomers, solvates, isotopically enriched analogs, prodrugs, or polymorphs thereof of the present disclosure by using the compound of Formula (I) or salts, stereoisomers (including enantiomers), solvates, isotopically enriched analogs, prodrugs, or polymorphs thereof.

In a further aspect, the present disclosure provides use of the compound of Formula (II) or salts, stereoisomers (including enantiomers), solvates, isotopically enriched analogs, prodrugs, or polymorphs thereof for the preparation of the compound of Formula (III) or salts, enantiomers, stereoisomers, solvates, isotopically enriched analogs, prodrugs, or polymorphs thereof.

In a further aspect, the present disclosure also provides a method for preparing the compound of Formula (III) or salts, enantiomers, stereoisomers, solvates, isotopically enriched analogs, prodrugs, or polymorphs thereof of the present disclosure by using the compound of Formula (II) or salts, stereoisomers (including enantiomers), solvates, isotopically enriched analogs, prodrugs, or polymorphs thereof.

In a further aspect, the present disclosure provides use of the compound of Formula (I) or salts, stereoisomers (including enantiomers), solvates, isotopically enriched analogs, prodrugs, or polymorphs thereof for the manufacture of a medicament for the prevention or treatment of cereblon protein-mediated disease or disorder.

In a further aspect, the present disclosure provides use of the compound of Formula (II) or salts, stereoisomers (including enantiomers), solvates, isotopically enriched analogs, prodrugs, or polymorphs thereof for the manufacture of a medicament for the prevention or treatment of cereblon protein-mediated disease or disorder.

In a further aspect, the present disclosure provides use of the compound of Formula (III) or salts, stereoisomers (including enantiomers), solvates, isotopically enriched analogs, prodrugs, or polymorphs thereof for the manufacture of a medicament for the prevention and/or treatment of a disease or disorder selected from the group consisting of tumor, infectious disease, autoinflammatory disease, inflammatory disease, autoimmune disease, neurological disease, respiratory disease, anemia, hemorrhagic shock, transplant rejection, multiple organ dysfunction syndrome (MODS), sarcoidosis, adult respiratory distress syndrome, congestive heart failure, myocardial infarction, Unverricht's syndrome, Richter syndrome (RS), acute liver failure, diabetes, Kennedy disease, seborrheic alopecia, hirsutism, skin disease, cardiovascular disease, dysfunctional uterine bleeding, anemia, pediatric aplastic anemia, endometriosis, transplant rejection, polycystic ovary syndrome, or thyroid disease.

In a further aspect, the present disclosure provides a method for treating or preventing a cereblon protein-mediated disease or disorder in a subject, comprising administering to the subject a therapeutically effective amount of the compound of Formula (I) or salts, enantiomers, stereoisomers, solvates, isotopically enriched analogs, prodrugs, or polymorphs thereof, or the pharmaceutical composition comprising the same.

In a further aspect, the present disclosure provides a method for treating or preventing a cereblon protein-mediated disease or disorder in a subject, comprising administering to the subject a therapeutically effective amount of the compound of Formula (II) or salts, enantiomers, stereoisomers, solvates, isotopically enriched analogs, prodrugs, or polymorphs thereof, or the pharmaceutical composition comprising the same.

In a further aspect, the present disclosure provides a method for treating or preventing a disease or disorder in a subject, comprising administering to the subject a therapeutically effective amount of the compound of Formula (III) or salts, enantiomers, stereoisomers, solvates, isotopically enriched analogs, prodrugs, or polymorphs thereof, or the pharmaceutical composition comprising the same, wherein the disease or disorder comprises tumor, infectious disease, autoinflammatory disease, inflammatory disease, autoimmune disease, neurological disease, respiratory disease, anemia, hemorrhagic shock, transplant rejection, multiple organ dysfunction syndrome (MODS), sarcoidosis, adult respiratory distress syndrome, congestive heart failure, myocardial infarction, Unverricht's syndrome, Richter syndrome (RS), acute liver failure, diabetes, Kennedy disease, seborrheic alopecia, hirsutism, skin disease, cardiovascular disease, dysfunctional uterine bleeding, anemia, pediatric aplastic anemia, endometriosis, transplant rejection, polycystic ovary syndrome, and thyroid disease.

### BRIEF DESCRIPTION OF DRAWINGS

FIG. 1 shows that the compounds of the present invention can effectively inhibit the proliferation of the tumor cell line DU-145, with a better inhibitory effect than the positive control enzalutamide.

FIG. 2 illustrates the degradation ability of the compounds of the present invention on full-length androgen receptor (AR-FL) and androgen receptor splice variant 7 (AR-V7) proteins in human prostate cancer 22RV1 cells, as determined by Western-blot.

FIG. 3 illustrates the degradation ability of the compounds of the present invention on full-length androgen receptor (AR-FL) protein in human prostate cancer C₄₋₂ cells, as determined by Western-blot.

### DETAILED DESCRIPTION OF THE INVENTION

The following detailed description is provided as exemplary specific embodiments to assist those skilled in the art in understanding and practicing the present disclosure. It should be appreciated, however, that such description is not intended to limit the scope of the present disclosure, and that various modifications and changes may be made to the specific embodiments described in the present disclosure without departing from the spirit and scope of the present disclosure. Such changes and modifications are to be understood as being included within the scope of the present invention as defined by the appended claims.

### I. Compounds

### Compounds of Formula (I)

The present disclosure provides a compound of Formula (I) or salts (including pharmaceutically acceptable salts), stereoisomers (including enantiomers), solvates, isotopically enriched analogs, prodrugs, or polymorphs thereof:

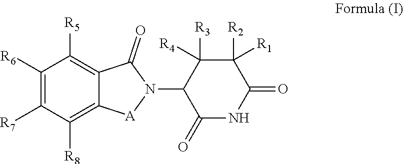

wherein A, R₁, R₂, R₃, R₄, R₅, R₆, R₇ and R₈ are as defined in the compounds of Formula (I) above.

In some embodiments, R₁, R₂, R₃ and R₄ of the compound of Formula (I) are the same or different and each independently represent hydrogen, deuterium, halogen (e.g., fluorine, chlorine, bromine, or iodine), optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—).

In some embodiments, R₁, R₂, R₃ and R₄ of the compound of Formula (I) are the same or different and each independently represent hydrogen or deuterium.

In some embodiments, R₁, R₂, R₃ and R₄ of the compound of Formula (I) represents hydrogen.

In some embodiments, A of the compound of Formula (I) represents CO.

In some embodiments, A of the compound of Formula (I) represents CH₂.

In some embodiments, A of the compound of Formula (I) represents CD₂.

In some embodiments, when two to four (e.g., 2, 3, or 4) of R₅, R₆, R₇ and R₈ of the compound of Formula (I) independently represent R₉, the compound of Formula (I) is optionally partially deuterated, for example, carrying 1 to 20 deuterium atoms (e.g., 1-15, 1-10, 1-6, 1-5, 1-4, 1-3, 1-2, or 1 deuterium atom), or the compound of Formula (I) is optionally fully deuterated. The number of deuterium atoms is not theoretically limited in any way or automatically limited by the size of the building units.

In some embodiments, when only one of R₅, R₆, R₇ and R₈ of the compound of Formula (I) represents R₉, the compound of Formula (I) is partially deuterated, for example, carrying 1 to 15 deuterium atoms (e.g., 1-10, 1-6, 1-5, 1-4, 1-3, 1-2, or 1 deuterium atom), or the compound of Formula (I) is fully deuterated. The number of deuterium atoms is not theoretically limited in any way or automatically limited by the size of the building units. In a sub-embodiment, when R₅ of the compound of Formula (I) represents R₉, R₆ and R₇ as well as R₈ are the same or different and each independently represent hydrogen, deuterium, halogen, hydroxy, mercapto, nitro, amino, cyano, optionally deuterated C₁₋₆ alkyl, optionally deuterated C₁₋₆ alkoxy, halogenated C₁₋₆ alkyl, C₂₋₆ alkenyl, or C₂₋₆ alkynyl. In a sub-embodiment, when R₆ of the compound of Formula (I) represents R₉, R₅ and R₇ as well as R₈ are the same or different and each independently represent hydrogen, deuterium, halogen, hydroxy, mercapto, nitro, amino, cyano, optionally deuterated C₁₋₆ alkyl, optionally deuterated C₁₋₆ alkoxy, halogenated C₁₋₆ alkyl, C₂₋₆ alkenyl, or C₂₋₆ alkynyl. In a sub-embodiment, when R₇ of the compound of Formula (I) represents R₉, R₅ and R₆ as well as R₈ are the same or different and each independently represent hydrogen, deuterium, halogen, hydroxy, mercapto, nitro, amino, cyano, optionally deuterated C₁₋₆ alkyl, optionally deuterated C₁₋₆ alkoxy, halogenated C₁₋₆ alkyl, C₂₋₆ alkenyl, or C₂₋₆ alkynyl. In a sub-embodiment, when R₈ of the compound of Formula (I) represents R₉, R₅ and R₆ as well as R₇ are the same or different and each independently represent hydrogen, deuterium, halogen, hydroxy, mercapto, nitro, amino, cyano, optionally deuterated C₁₋₆ alkyl, optionally deuterated C₁₋₆ alkoxy, halogenated C₁₋₆ alkyl, C₂₋₆ alkenyl, or C₂₋₆ alkynyl.

In some embodiments, in case that only one of R₅, R₆, R₇ and R₈ of the compound of Formula (I) represents R₉ and the compound of Formula (I) is not deuterated, when R represents optionally substituted nitrogen-containing bridged heterocyclyl, the compound of Formula (I) does not comprise the following compounds:
5-(2,5-diazabicyclo[2.2.1]heptan-2-yl)-2-(2,6-dioxopyridin-3-yl)isoindoline-1,3-dione;
5-((1R,4R)-2,5-diazabicyclo[2.2.1]heptan-2-yl)-2-(2,6-dioxopyridin-3-yl)-6-fluoroisoindoline-1,3-dione;
5-(2,5-diazabicyclo[2.2.1]heptan-2-yl)-2-(2,6-dioxopyridin-3-yl)-6-fluoroisoindoline-1,3-dione;
3-(5-(2,5-diazabicyclo[2.2.1]heptan-2-yl)-1-oxoisoindolin-2-yl)piperidine-2,6-dione;
3-(5-(8-azabicyclo[3.2.1]octan-3-yl)-1-oxoisoindolin-2-yl)piperidine-2,6-dione; and
3-(5-(9-ethyl-3-methyl-3,9-diazabicyclo[3.3.1]nonan-7-yl)-1-oxoisoindolin-2-yl)piperidine-2,6-dione.

In some embodiments, in case that only one of R₅, R₆, R₇ and R₈ of the compound of Formula (I) represents R₉ and the compound of Formula (I) is not deuterated, when A represents CO and one of R₆ and R₇ represents R₉, where R represents substituted nitrogen-containing monocyclic heterocyclyl, and the other one of R₆ and R₇, as well as R₅ and R₈ each independently represent hydrogen, halogen (e.g., fluorine, chlorine, bromine, or iodine), hydroxy, mercapto, nitro, amino, cyano, C₁₋₆ alkyl (e.g., C₁₋₄ alkyl, such as methyl, ethyl, propyl, isopropyl, butyl, sec-butyl or tert-butyl), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₂₋₆ alkenyl (e.g., vinyl, propenyl, or butenyl), or C₂₋₆ alkynyl (e.g., ethynyl, propynyl, or butynyl), one to three (e.g., 1, 2 or 3) of the other one of R₆ and R₇ as well as R₅ and R₈ represents halogen, and the nitrogen-containing monocyclic heterocyclyl is substituted with one or more (e.g., 1-6, such as 1, 2, 3, 4, 5 or 6) substituents selected from the group consisting of hydroxy, amino, mercapto, nitro, halogen (e.g., fluorine, chlorine, bromine, or iodine), cyano, C₁₋₆ alkyl (e.g., C₁₋₄ alkyl, such as methyl, ethyl, propyl, isopropyl, butyl, sec-butyl or tert-butyl), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), NH₂—C₁₋₆ alkylene (e.g., NH₂—C₁₋₃ alkylene-, such as NH₂CH₂—, NH₂CH₂CH₂— and NH₂CH₂CH₂CH₂—), C₁₋₆ alkyl-NH— (e.g., C₁₋₃ alkyl-NH—, such as CH₃NH—, CH₃CH₂NH— or CH₃CH₂CH₂NH—), C₁₋₆ alkyl-NHC(O)— (e.g., CH₃—NHC(O)—, CH₃CH₂—NHC(O)—, and CH₃CH₂CH₂—NHC(O)—), C₁₋₆ alkyl-C(O)NH— (e.g., CH₃—C(O)NH—, CH₃CH₂—C(O)NH—, and CH₃CH₂CH₂—C(O)NH—) or any combination thereof. The number of substituents is not theoretically limited in any way or automatically limited by the size of the building units.

In some embodiments, in case that only one of R₅, R₆, R₇ and R₈ of the compound of Formula (I) represents R₉ and the compound of Formula (I) is not deuterated, when A represents CO and one of R₆ and R₇ represents R₉, where R represents unsubstituted nitrogen-containing monocyclic heterocyclyl, the other one of R₆ and R₇ represents hydrogen, halogen (e.g., fluorine, chlorine, bromine, or iodine), hydroxy, mercapto, nitro, amino, cyano, C₁₋₆ alkyl (e.g., C₁₋₄ alkyl, such as methyl, ethyl, propyl, isopropyl, butyl, sec-butyl or tert-butyl), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₂₋₆ alkenyl (e.g., vinyl, propenyl, or butenyl), or C₂₋₆ alkynyl (e.g., ethynyl, propynyl, or butynyl), and R₅ and R₈ each independently represent halogen (e.g., fluorine, chlorine, bromine, or iodine).

In some embodiments, in case that only one of R₅, R₆, R₇ and R₈ of the compound of Formula (I) represents R₉ and the compound of Formula (I) is not deuterated, when A represents CO and one of R₅ and R₈ represents R₉, where R represents substituted nitrogen-containing monocyclic heterocyclyl, and the other one of R₅ and R₈, as well as R₆ and R₇ each independently represent hydrogen, halogen (e.g., fluorine, chlorine, bromine, or iodine), hydroxy, mercapto, nitro, amino, cyano, C₁₋₆ alkyl (e.g., C₁₋₄ alkyl, such as methyl, ethyl, propyl, isopropyl, butyl, sec-butyl or tert-butyl), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₂₋₆ alkenyl (e.g., vinyl, propenyl, or butenyl), or C₂₋₆ alkynyl (e.g., ethynyl, propynyl, or butynyl), one to three (e.g., 1, 2 or 3) of the other one of R₅ and R₈ as well as R₆ and R₇ represents halogen (e.g., fluorine, chlorine, bromine, or iodine), and the nitrogen-containing monocyclic heterocyclyl is substituted with one or more (e.g., 1-6, such as 1, 2, 3, 4, 5 or 6) substituents selected from the group consisting of hydroxy, amino, mercapto, nitro, halogen (e.g., fluorine, chlorine, bromine, or iodine), cyano, CHO, C₁₋₆ alkyl (e.g., C₁₋₄ alkyl, such as methyl, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₃₋₆ cycloalkyl (e.g., cyclopropyl, cyclobutyl, cyclopentyl, or cyclohexyl), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl-NH— (e.g., C₁₋₃ alkyl-NH—, such as CH₃NH—, CH₃CH₂NH— or CH₃CH₂CH₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), C₁₋₆ alkyl-NHC(O)— (e.g., CH₃—NHC(O)—, CH₃CH₂—NHC(O)—, and CH₃CH₂CH₂—NHC(O)—), C₁₋₆ alkyl-C(O)NH— (e.g., CH₃—C(O)NH—, CH₃CH₂—C(O)NH—, and CH₃CH₂CH₂—C(O)NH—) or any combination thereof. The number of substituents is not theoretically limited in any way or automatically limited by the size of the building units.

In some embodiments, in case that only one of R₅, R₆, R₇ and R₈ of the compound of Formula (I) represents R₉ and the compound of Formula (I) is not deuterated, when A represents CO and one of R₅ and R₈ represents R₉, where R represents unsubstituted nitrogen-containing monocyclic heterocyclyl, the other one of R₅ and R₈ and/or R₇ each independently represent halogen, and R₆ represents hydrogen, halogen (e.g., fluorine, chlorine, bromine, or iodine), hydroxy, mercapto, nitro, amino, cyano, C₁₋₆ alkyl (e.g., C₁₋₄ alkyl, such as methyl, ethyl, propyl, isopropyl, butyl, sec-butyl or tert-butyl), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₂₋₆ alkenyl (e.g., vinyl, propenyl, or butenyl), or C₂₋₆ alkynyl (e.g., ethynyl, propynyl, or butynyl).

In some embodiments, in case that only one of R₅, R₆, R₇ and R₈ of the compound of Formula (I) represents R₉ and the compound of Formula (I) is not deuterated, when A represents CH₂ and one of R₆ and R₇ represents R₉, where R represents substituted nitrogen-containing monocyclic heterocyclyl, and the other one of R₆ and R₇, as well as R₅ and R₈ each independently represent hydrogen, halogen (e.g., fluorine, chlorine, bromine, or iodine), hydroxy, mercapto, nitro, amino, cyano, C₁₋₆ alkyl (e.g., C₁₋₄ alkyl, such as methyl, ethyl, propyl, isopropyl, butyl, sec-butyl or tert-butyl), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₂₋₆ alkenyl (e.g., vinyl, propenyl, or butenyl), or C₂₋₆ alkynyl (e.g., ethynyl, propynyl, or butynyl), one to three (e.g., 1, 2 or 3) of the other one of R₆ and R₇ as well as R₅ and R₈ represents halogen (e.g., fluorine, chlorine, bromine, or iodine), and the nitrogen-containing monocyclic heterocyclyl is substituted with one or more (e.g., 1-6, such as 1, 2, 3, 4, 5 or 6) substituents selected from the group consisting of hydroxy, amino, mercapto, nitro, halogen (e.g., fluorine, chlorine, bromine, or iodine), CHO, cyano, C₁₋₆ alkyl (e.g., C₁₋₄ alkyl, such as methyl, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₃₋₆ cycloalkyl (e.g., cyclopropyl, cyclobutyl, cyclopentyl, or cyclohexyl), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl-NH— (e.g., C₁₋₃ alkyl-NH—, such as CH₃NH—, CH₃CH₂NH— or CH₃CH₂CH₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), C₁₋₆ alkyl-NHC(O)— (e.g., CH₃—NHC(O)—, CH₃CH₂—NHC(O)—, and CH₃CH₂CH₂—NHC(O)—), C₁₋₆ alkyl-C(O)NH— (e.g., CH₃—C(O)NH—, CH₃CH₂—C(O)NH—, and CH₃CH₂CH₂—C(O)NH—) or any combination thereof. The number of substituents is not theoretically limited in any way or automatically limited by the size of the building units.

In some embodiments, in case that only one of R₅, R₆, R₇ and R₈ of the compound of Formula (I) represents R₉ and the compound of Formula (I) is not deuterated, when A represents CH₂ and one of R₆ and R₇ represents R₉, where R represents unsubstituted nitrogen-containing monocyclic heterocyclyl, R₅ represents halogen (e.g., fluorine, chlorine, bromine, or iodine), and the other one of R₆ and R₇ and/or R₉ each independently represent halogen (e.g., fluorine, chlorine, bromine, or iodine), hydroxy, mercapto, nitro, amino, cyano, C₁₋₆ alkyl (e.g., C₁₋₄ alkyl, such as methyl, ethyl, propyl, isopropyl, butyl, sec-butyl or tert-butyl), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₂₋₆ alkenyl (e.g., vinyl, propenyl, or butenyl), or C₂₋₆ alkynyl (e.g., ethynyl, propynyl, or butynyl).

In some embodiments, in case that only one of R₅, R₆, R₇ and R₈ of the compound of Formula (I) represents R₉ and the compound of Formula (I) is not deuterated, when A represents CH₂ and one of R₅ and R₈ represents R₉, where R represents substituted nitrogen-containing monocyclic heterocyclyl, and the other one of R₅ and R₈, as well as R₆ and R₇ each independently represent hydrogen, halogen (e.g., fluorine, chlorine, bromine, or iodine), hydroxy, mercapto, nitro, amino, cyano, C₁₋₆ alkyl (e.g., C₁₋₄ alkyl, such as methyl, ethyl, propyl, isopropyl, butyl, sec-butyl or tert-butyl), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₂₋₆ alkenyl (e.g., vinyl, propenyl, or butenyl), or C₂₋₆ alkynyl (e.g., ethynyl, propynyl, or butynyl), one to three (e.g., 1, 2 or 3) of the other one of R₅ and R₈ as well as R₆ and R₇ represents halogen (e.g., fluorine, chlorine, bromine, or iodine), and the nitrogen-containing monocyclic heterocyclyl is substituted with one or more (e.g., 1-6, such as 1, 2, 3, 4, 5 or 6) substituents selected from the group consisting of hydroxy, amino, mercapto, nitro, halogen (e.g., fluorine, chlorine, bromine, or iodine), CHO, cyano, C₁₋₆ alkyl (e.g., C₁₋₄ alkyl, such as methyl, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₃₋₆ cycloalkyl (e.g., cyclopropyl, cyclobutyl, cyclopentyl, or cyclohexyl), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl-NH— (e.g., C₁₋₃ alkyl-NH—, such as CH₃NH—, CH₃CH₂NH— or CH₃CH₂CH₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), C₁₋₆ alkyl-NHC(O)— (e.g., CH₃—NHC(O)—, CH₃CH₂—NHC(O)—, and CH₃CH₂CH₂—NHC(O)—), C₁₋₆ alkyl-C(O)NH— (e.g., CH₃—C(O)NH—, CH₃CH₂—C(O)NH—, and CH₃CH₂CH₂—C(O)NH—) or any combination thereof. The number of substituents is not theoretically limited in any way or automatically limited by the size of the building units.

In some embodiments, in case that only one of R₅, R₆, R₇ and R₈ of the compound of Formula (I) represents R₉ and the compound of Formula (I) is not deuterated, when A represents CH₂ and one of R₅ and R₅ represents R₉, where R represents unsubstituted nitrogen-containing monocyclic heterocyclyl, and the other one of R₅ and R₈, as well as R₆ and R₇ each independently represent hydrogen, halogen (e.g., fluorine, chlorine, bromine, or iodine), hydroxy, mercapto, nitro, amino, cyano, C₁₋₆ alkyl (e.g., C₁₋₄ alkyl, such as methyl, ethyl, propyl, isopropyl, butyl, sec-butyl or tert-butyl), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₂₋₆ alkenyl (e.g., vinyl, propenyl, or butenyl), or C₂₋₆ alkynyl (e.g., ethynyl, propynyl, or butynyl), the other one of R₅ and R₈, as well as R₆ and R₇ are not simultaneously hydrogen, and when R₆ is halogen (e.g., fluorine, chlorine, bromine, or iodine), the other one of R₅ and R₈ as well as R₇ are not simultaneously hydrogen.

In some embodiments, the group R of R₉ of the compound of Formula (I) represents optionally substituted nitrogen-containing heterocyclyl, which is optionally substituted nitrogen-containing monocyclic heterocyclyl or optionally substituted nitrogen-containing bridged heterocyclyl. In a sub-embodiment, the nitrogen-containing monocyclic heterocyclyl is a 4- to 10-membered monocyclic heterocyclyl group containing one nitrogen atom and optionally a heteroatom selected from nitrogen, oxygen, and sulfur, including but not limited to the following groups: piperidinyl, piperazinyl, morpholinyl, azetidinyl, pyrrolidinyl, imidazolidinyl, pyrazolidyl, oxazolidinyl, thiazolidinyl, thiomorpholinyl, azepanyl, diazacycloheptanyl, azacyclooctyl or diazacyclooctyl. In a sub-embodiment, the nitrogen-containing bridged heterocyclyl is a C₅₋₂₀ bridged heterocyclyl group containing one nitrogen atom and optionally a heteroatom selected from nitrogen, oxygen, and sulfur, including but not limited to the following groups: azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl or diazabicyclo[2.2.2]octanyl, such as the following groups:

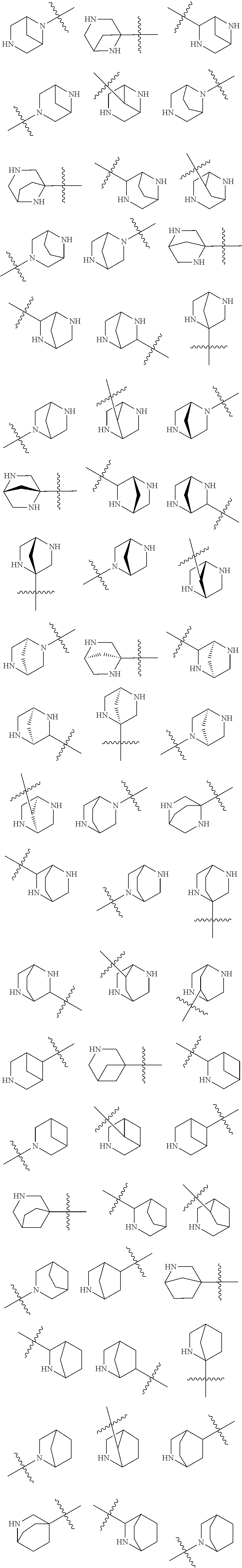

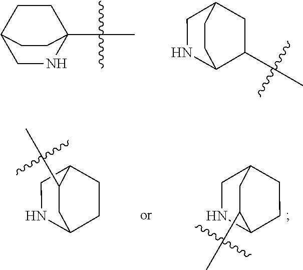 [SMILES: *C12CCC(CC1)CN2 ; *C1CC2CCC1NC2 ; *C1CC2CCC1CN2 ; "or" ; *C1CC2CCC1NC2 ; ";"]

wherein the nitrogen-containing monocyclic heterocyclyl and nitrogen-containing bridged heterocyclyl are optionally substituted. In a sub-embodiment, the nitrogen-containing monocyclic heterocyclyl and nitrogen-containing bridged heterocyclyl are optionally substituted with one or more (e.g., 1-10, 1-8 or 1-6, such as 1, 2, 3, 4, 5 or 6) substituents selected from the group consisting of deuterium, hydroxy, amino, mercapto, nitro, halogen (e.g., fluorine, chlorine, bromine, or iodine), cyano, CHO, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally deuterated cyclopropyl, cyclobutyl, cyclopentyl, or cyclohexyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., C₁₋₃ alkyl-NH—, such as CH₃NH—, CH₃CH₂NH— or CH₃CH₂CH₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), C₁₋₆ alkyl-NHC(O)— (e.g., CH₃—NHC(O)—, CH₃CH₂—NHC(O)—, and CH₃CH₂CH₂—NHC(O)—), C₁₋₆ alkyl-C(O)NH— (e.g., CH₃—C(O)NH—, CH₃CH₂—C(O)NH—, and CH₃CH₂CH₂—C(O)NH—) or any combination thereof.

In some embodiments, R₉ of the compound of Formula (I) represents the following groups:

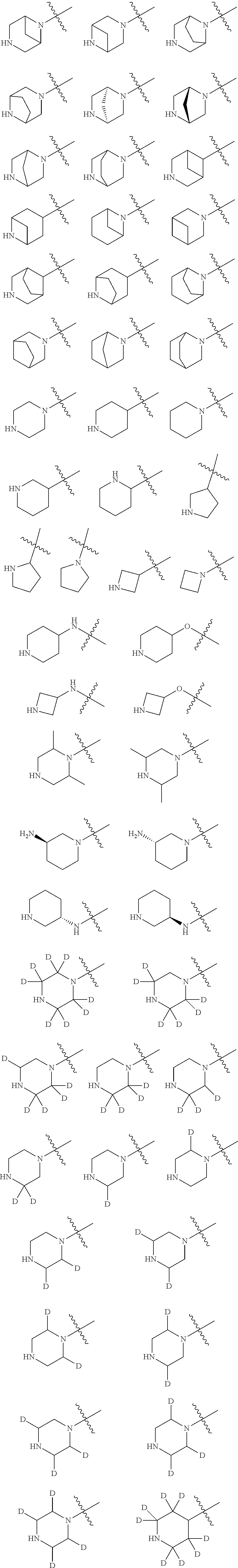

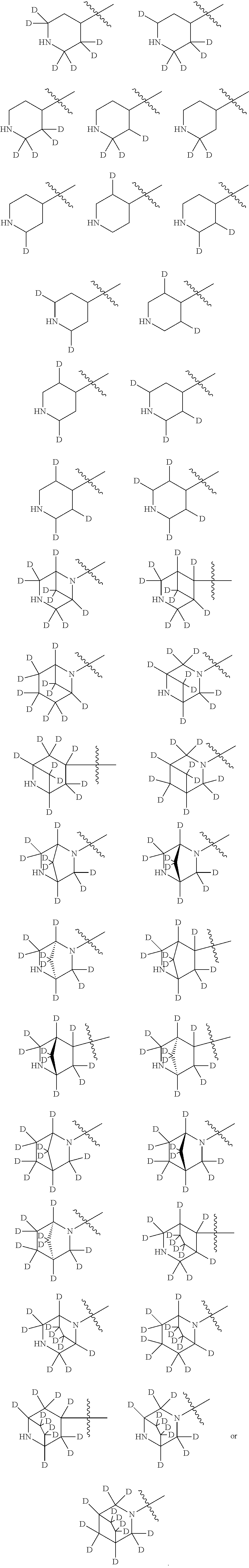

In some embodiments, the compound of Formula (I) is also of Formula (I-1) or Formula (I-2):

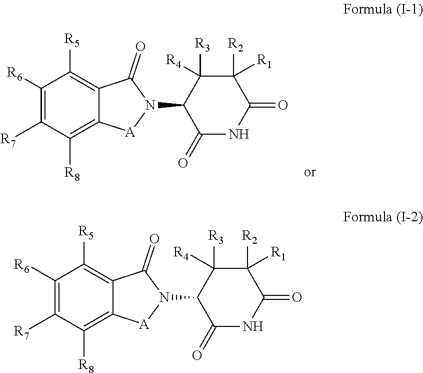

wherein A, R₁, R₂, R₃, R₄, R₅, R₆, R₇ and R₈ are as defined in the compound of Formula (I) above.

In some embodiments, the compound of Formula (I) is also of Formula (I-3), Formula (I-4) or Formula (I-5):

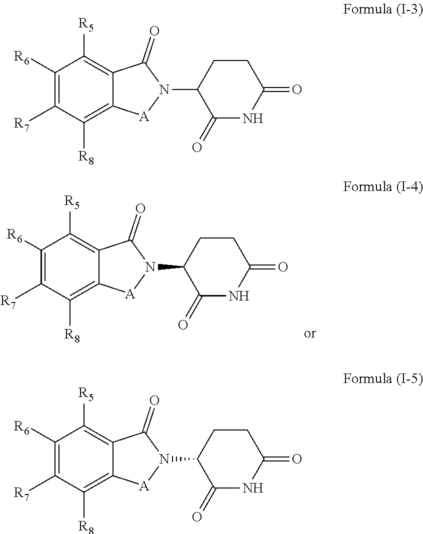

wherein A, R₅, R₆, R₇ and R₈ are as defined in the compound of Formula (I) above.

In some embodiments, the compounds of Formula (I) of the present invention and their salts (including pharmaceutically acceptable salts, such as hydrochloride, etc.), prodrugs, solvates, isotopically enriched analogs, polymorphs, stereoisomers (including enantiomers and diastereomers), or mixture of stereoisomers thereof in Table 1 below are provided.

TABLE 1
The compounds of the present disclosure

| Compound | | |
|---|---|---|
| No. | Structure of compound | Compound's name |
| GT-03100 | 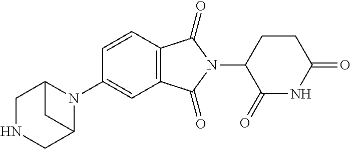 [SMILES: O=C1CCC(N2C(=O)c3ccc(N4C5CNCC4C5)cc3C2=O)C(=O)N1] | 5-(3,6-diazabicyclo[3.1.1]heptan-6-yl)-2- (2,6-dioxopiperidin- 3-yl)isoindoline-1,3-dione |
| GT-03075 | 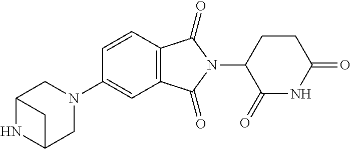 [SMILES: O=C1CCC(N2C(=O)c3ccc(N4CC5CC(C4)N5)cc3C2=O)C(=O)N1] | 5-(3,6-diazabicyclo[3.1.1]heptan-3-yl)- 2-(2,6-dioxopiperidin- 3-yl)isoindoline-1,3-dione |
| GT-03210 | 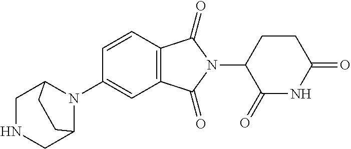 [SMILES: O=C1CCC(N2C(=O)c3ccc(N4C5CCC4CNC5)cc3C2=O)C(=O)N1] | 5-(3,8-diazabicyclo[3.2.1]octan-8-yl)- 2-(2,6-dioxopiperidin-3- yl)isoindoline-1,3-dione |
| GT-03077 | 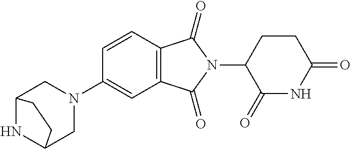 [SMILES: O=C1CCC(N2C(=O)c3ccc(N4CC5CCC(C4)N5)cc3C2=O)C(=O)N1] | 5-(3,8-diazabicyclo[3.2.1]octan-3-yl)- 2-(2,6-dioxopiperidin-3- yl)isoindoline-1,3-dione |
| GT-03072 | 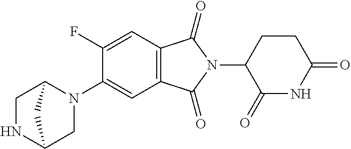 [SMILES: O=C1CCC(N2C(=O)c3cc(F)c(N4C[C@@H]5C[C@H]4CN5)cc3C2=O)C(=O)N1] | 5-((1S,4S)-2,5-diazabicyclo[2.2.1]heptan- 2-yl)-2-(2,6- dioxopiperidin-3-yl)- 6-fluoroisoindoline-1,3-dione |
| GT-03076 | 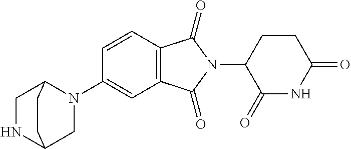 [SMILES: O=C1CCC(N2C(=O)c3ccc(N4CC5CCC4CN5)cc3C2=O)C(=O)N1] | 5-(2,5-diazabicyclo[2.2.2]octan-2-yl)- 2-(2,6-dioxopiperidin-3- yl)isoindoline-1,3-dione |
| | 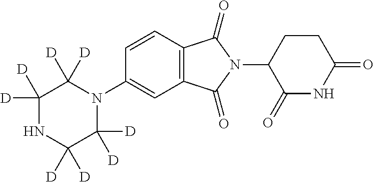 [SMILES: [2H]C1([2H])NC([2H])([2H])C([2H])([2H])N(c2ccc3c(c2)C(=O)N(C2CCC(=O)NC2=O)C3=O)C1([2H])[2H]] | 2-(2,6-dioxopiperidin-3-yl)-5- (piperazin-1-yl-2,2,3,3,5,5,6,6- d₈)isoindoline-1,3-dione |
| | 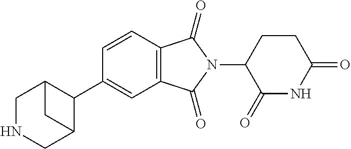 [SMILES: O=C1CCC(N2C(=O)c3ccc(C4C5CNCC4C5)cc3C2=O)C(=O)N1] | 5-(3-azabicyclo[3.1.1]heptan-6-yl)- 2-(2,6-dioxopiperidin-3- yl)isoindoline-1,3-dione |
| | 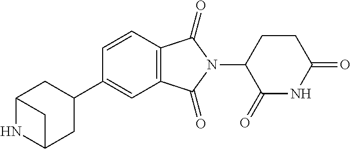 [SMILES: O=C1CCC(N2C(=O)c3ccc(C4CC5CC(C4)N5)cc3C2=O)C(=O)N1] | 5-(6-azabicyclo[3.1.1]heptan-3-yl)- 2-(2,6-dioxopiperidin-3- yl)isoindoline-1,3-dione |
| | 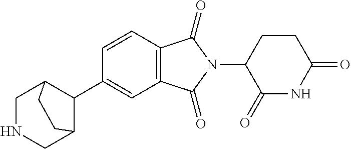 [SMILES: O=C1CCC(N2C(=O)c3ccc(C4C5CCC4CNC5)cc3C2=O)C(=O)N1] | 5-(3-azabicyclo[3.2.1]octan-8-yl)- 2-(2,6-dioxopiperidin-3- yl)isoindoline-1,3-dione |
| | 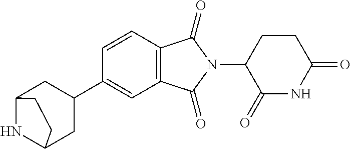 [SMILES: O=C1CCC(N2C(=O)c3ccc(C4CC5CCC(C4)N5)cc3C2=O)C(=O)N1] | 5-(8-azabicyclo[3.2.1]octan-3-yl)- 2-(2,6-dioxopiperidin-3- yl)isoindoline-1,3-dione |
| | 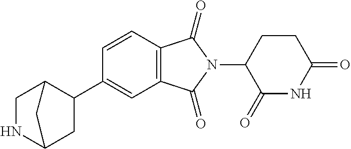 [SMILES: O=C1CCC(N2C(=O)c3ccc(C4CC5CC4CN5)cc3C2=O)C(=O)N1] | 5-(2-azabicyclo[2.2.1]heptan-5-yl)- 2-(2,6-dioxopiperidin-3- yl)isoindoline-1,3-dione |
| | 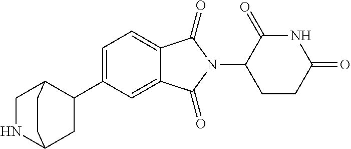 [SMILES: O=C1CCC(N2C(=O)c3ccc(C4CC5CCC4CN5)cc3C2=O)C(=O)N1] | 5-(2-azabicyclo[2.2.2]octan-5-yl)- 2-(2,6-dioxopiperidin-3- yl)isoindoline-1,3-dione |
| GT-03106 | 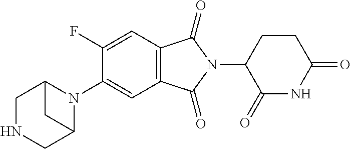 [SMILES: O=C1CCC(N2C(=O)c3cc(F)c(N4C5CNCC4C5)cc3C2=O)C(=O)N1] | 5-(3,6-diazabicyclo[3.1.1]heptan-6-yl)- 2-(2,6-dioxopiperidin- 3-yl)-6-fluoroisoindoline-1,3-dione |
| GT-03109 | 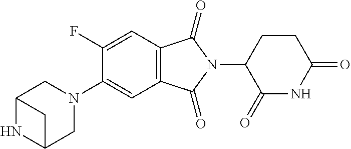 [SMILES: O=C1CCC(N2C(=O)c3cc(F)c(N4CC5CC(C4)N5)cc3C2=O)C(=O)N1] | 5-(3,6-diazabicyclo[3.1.1]heptan-3-yl)- 2-(2,6-dioxopiperidin- 3-yl)-6-fluoroisoindoline-1,3-dione |
| GT-03211 | 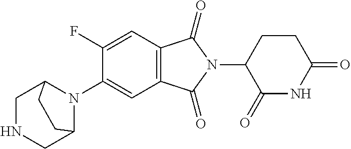 [SMILES: O=C1CCC(N2C(=O)c3cc(F)c(N4C5CCC4CNC5)cc3C2=O)C(=O)N1] | 5-(3,8-diazabicyclo[3.2.1]octan-8-yl)- 2-(2,6-dioxopiperidin-3- yl)-6-fluoroisoindoline-1,3-dione |
| GT-03107 | 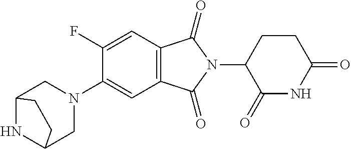 [SMILES: O=C1CCC(N2C(=O)c3cc(F)c(N4CC5CCC(C4)N5)cc3C2=O)C(=O)N1] | 5-(3,8-diazabicyclo[3.2.1]octan-3-yl)- 2-(2,6-dioxopiperidin-3- yl)-6-fluoroisoindoline-1,3-dione |
| GT-03103 | 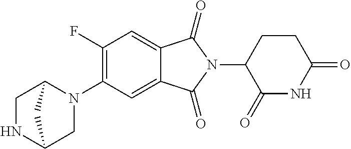 [SMILES: O=C1CCC(N2C(=O)c3cc(F)c(N4C[C@@H]5C[C@H]4CN5)cc3C2=O)C(=O)N1] | 5-((1S,4S)-2,5-diazabicyclo[2.2.1]heptan-2-yl)- 2-(2,6-dioxopiperidin-3-yl)- 6-fluoroisoindoline-1,3-dione |
| GT-03105 | 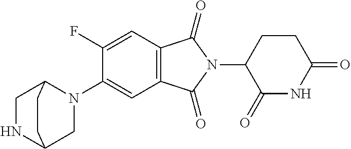 [SMILES: O=C1CCC(N2C(=O)c3cc(F)c(N4CC5CCC4CN5)cc3C2=O)C(=O)N1] | 5-(2,5-diazabicyclo[2.2.2]octan-2-yl)- 2-(2,6-dioxopiperidin-3- yl)-6-fluoroisoindoline-1,3-dione |
| | 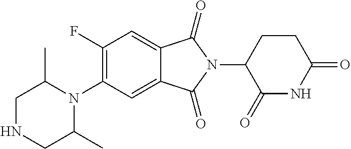 [SMILES: CC1CNCC(C)N1c1cc2c(cc1F)C(=O)N(C1CCC(=O)NC1=O)C2=O] | 5-(2,6-dimethylpiperazin-1-yl)- 2-(2,6-dioxopiperidin-3-yl)-6- fluoroisoindoline-1,3-dione |
| GT-03104 | 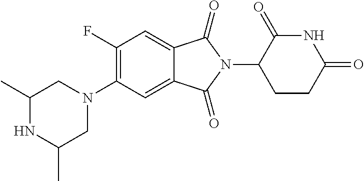 [SMILES: CC1CN(c2cc3c(cc2F)C(=O)N(C2CCC(=O)NC2=O)C3=O)CC(C)N1] | 5-(3,5-dimethylpiperazin-1-yl)- 2-(2,6-dioxopiperidin-3-yl)-6- fluoroisoindoline-1,3-dione |
| GT-03108 | 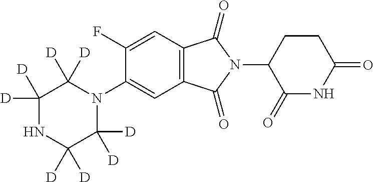 [SMILES: [2H]C1([2H])NC([2H])([2H])C([2H])([2H])N(c2cc3c(cc2F)C(=O)N(C2CCC(=O)NC2=O)C3=O)C1([2H])[2H]] | 2-(2,6-dioxopiperidin-3-yl)- 5-fluoro-6-(piperazin-1-yl- 2,2,3,3,5,5,6,6-d₈)isoindoline- 1,3-dione |
| | 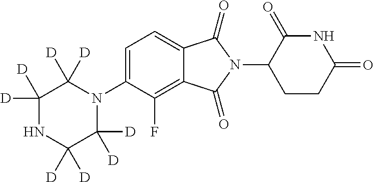 [SMILES: [2H]C1([2H])NC([2H])([2H])C([2H])([2H])N(c2ccc3c(c2F)C(=O)N(C2CCC(=O)NC2=O)C3=O)C1([2H])[2H]] | 2-(2,6-dioxopiperidin-3-yl)- 4-fluoro-5-(piperazin-1-yl- 2,2,3,3,5,5,6,6-d₈)isoindoline- 1,3-dione |
| | 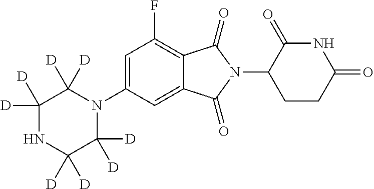 [SMILES: [2H]C1([2H])NC([2H])([2H])C([2H])([2H])N(c2cc(F)c3c(c2)C(=O)N(C2CCC(=O)NC2=O)C3=O)C1([2H])[2H]] | 2-(2,6-dioxopiperidin-3-yl)-4- fluoro-6-(piperazin-1-yl- 2,2,3,3,5,5,6,6-d₈)isoindoline- 1,3-dione |
| | 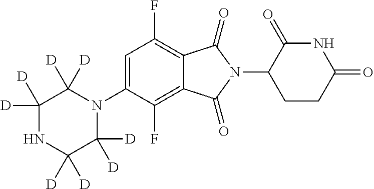 [SMILES: [2H]C1([2H])NC([2H])([2H])C([2H])([2H])N(c2cc(F)c3c(c2F)C(=O)N(C2CCC(=O)NC2=O)C3=O)C1([2H])[2H]] | 2-(2,6-dioxopiperidin-3-yl)- 4,7-difluoro-5-(piperazin-1-yl- 2,2,3,3,5,5,6,6-d₈)isoindoline- 1,3-dione |
| | 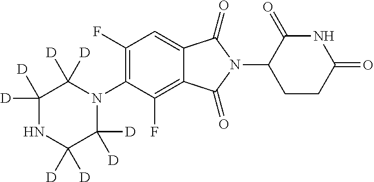 [SMILES: [2H]C1([2H])NC([2H])([2H])C([2H])([2H])N(c2c(F)cc3c(c2F)C(=O)N(C2CCC(=O)NC2=O)C3=O)C1([2H])[2H]] | 2-(2,6-dioxopiperidin-3-yl)- 4,6-difluoro-5-(piperazin-1-yl- 2,2,3,3,5,5,6,6-d₈)isoindoline- 1,3-dione |
| | 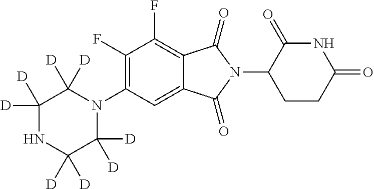 [SMILES: [2H]C1([2H])NC([2H])([2H])C([2H])([2H])N(c2cc3c(c(F)c2F)C(=O)N(C2CCC(=O)NC2=O)C3=O)C1([2H])[2H]] | 2-(2,6-dioxopiperidin-3-yl)- 4,5-difluoro-6-(piperazin-1-yl- 2,2,3,3,5,5,6,6-d₈)isoindoline- 1,3-dione |
| | 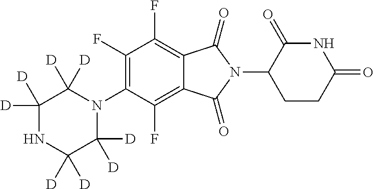 [SMILES: [2H]C1([2H])NC([2H])([2H])C([2H])([2H])N(c2c(F)c(F)c3c(c2F)C(=O)N(C2CCC(=O)NC2=O)C3=O)C1([2H])[2H]] | 2-(2,6-dioxopiperidin-3-yl)- 4,5,7-trifluoro-6-(piperazin-1-yl- 2,2,3,3,5,5,6,6-d₈)isoindoline- 1,3-dione |
| | 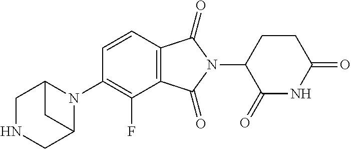 [SMILES: O=C1CCC(N2C(=O)c3ccc(N4C5CNCC4C5)c(F)c3C2=O)C(=O)N1] | 5-(3,6-diazabicyclo[3.1.1]heptan-6-yl)- 2-(2,6-dioxopiperidin- 3-yl)-4,fluoroisoindoline- 1,3-dione |
| | 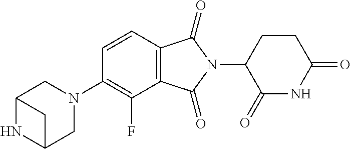 [SMILES: O=C1CCC(N2C(=O)c3ccc(N4CC5CC(C4)N5)c(F)c3C2=O)C(=O)N1] | 5-(3,6-diazabicyclo[3.1.1]heptan-3-yl)- 2-(2,6-dioxopiperidin- 3-yl)-4-fluoroisoindoline-1,3-dione |
| | 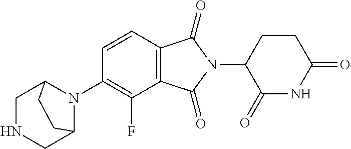 [SMILES: O=C1CCC(N2C(=O)c3ccc(N4C5CCC4CNC5)c(F)c3C2=O)C(=O)N1] | 5-(3,8-diazabicyclo[3.2.1]octan-8-yl)- 2-(2,6-dioxopiperidin-3- yl)-4-fluoroisoindoline-1,3-dione |
| | 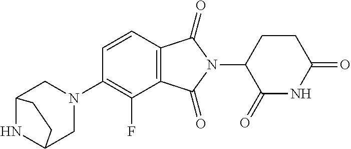 [SMILES: O=C1CCC(N2C(=O)c3ccc(N4CC5CCC(C4)N5)c(F)c3C2=O)C(=O)N1] | 5-(3,8-diazabicyclo[3.2.1]octan-3-yl)- 2-(2,6-dioxopiperidin-3- yl)-4-fluoroisoindoline-1,3-dione |
| | 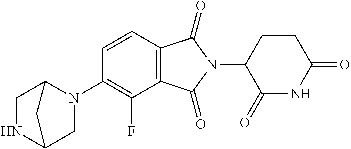 [SMILES: O=C1CCC(N2C(=O)c3ccc(N4CC5CC4CN5)c(F)c3C2=O)C(=O)N1] | 5-(2,5-diazabicyclo[2.2.1]heptan-2-yl)- 2-(2,6-dioxopiperidin- 3-yl)-4-fluoroisoindoline-1,3-dione |
| | 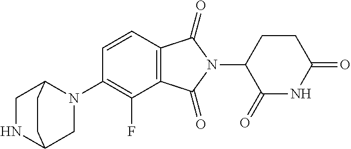 [SMILES: O=C1CCC(N2C(=O)c3ccc(N4CC5CCC4CN5)c(F)c3C2=O)C(=O)N1] | 5-(2,5-diazabicyclo[2.2.2]octan-2-yl)- 2-(2,6-dioxopiperidin-3- yl)-4,7-difluoroisoindoline-1,3-dione |
| | 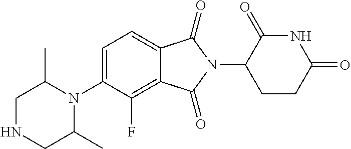 [SMILES: CC1CNCC(C)N1c1ccc2c(c1F)C(=O)N(C1CCC(=O)NC1=O)C2=O] | 5-(2,6-dimethylpiperazin-1-yl)- 2-(2,6-dioxopiperidin-3-yl)-4- fluoroisoindoline-1,3-dione |
| | 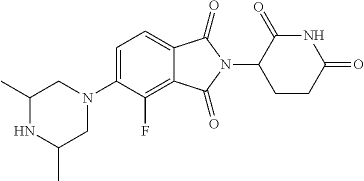 [SMILES: CC1CN(c2ccc3c(c2F)C(=O)N(C2CCC(=O)NC2=O)C3=O)CC(C)N1] | 5-(3,5-dimethylpiperazin-1-yl)- 2-(2,6-dioxopiperidin-3-yl)- 4-fluoroisoindoline-1,3-dione |
| | 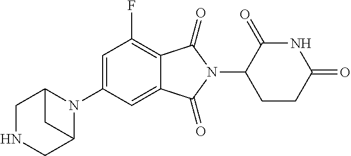 [SMILES: O=C1CCC(N2C(=O)c3cc(N4C5CNCC4C5)cc(F)c3C2=O)C(=O)N1] | 6-(3,6-diazabicyclo[3.1.1]heptan-6-yl)- 2-(2,6-dioxopiperidin- 3-yl)-4-fluoroisoindoline-1,3-dione |
| | 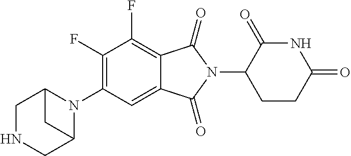 [SMILES: O=C1CCC(N2C(=O)c3cc(N4C5CNCC4C5)c(F)c(F)c3C2=O)C(=O)N1] | 6-(3,6-diazabicyclo[3.1.1]heptan-6-yl)- 2-(2,6-dioxopiperidin- 3-yl)-4,5-difluoroisoindoline-1,3-dione |
| | 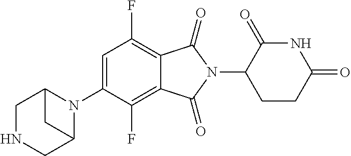 [SMILES: O=C1CCC(N2C(=O)c3c(F)cc(N4C5CNCC4C5)c(F)c3C2=O)C(=O)N1] | 5-(3,6-diazabicyclo[3.1.1]heptan-6-yl)- 2-(2,6-dioxopiperidin- 3-yl)-4,7-difluoroisoindoline-1,3-dione |
| | 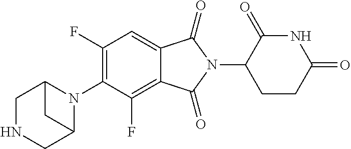 [SMILES: O=C1CCC(N2C(=O)c3cc(F)c(N4C5CNCC4C5)c(F)c3C2=O)C(=O)N1] | 5-(3,6-diazabicyclo[3.1.1]heptan-6-yl)- 2-(2,6-dioxopiperidin- 3-yl)-4,6-difluoroisoindoline-1,3-dione |
| | 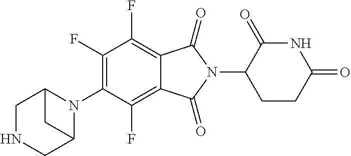 [SMILES: O=C1CCC(N2C(=O)c3c(F)c(F)c(N4C5CNCC4C5)c(F)c3C2=O)C(=O)N1] | 5-(3,6-diazabicyclo[3.1.1]heptan-6-yl)- 2-(2,6-dioxopiperidin-3-yl)- 4,6,7-trifluoroisoindoline-1,3-dione |
| | 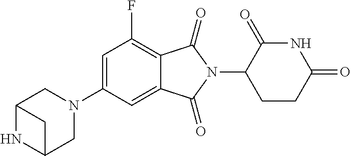 [SMILES: O=C1CCC(N2C(=O)c3cc(N4CC5CC(C4)N5)cc(F)c3C2=O)C(=O)N1] | 6-(3,6-diazabicyclo[3.1.1]heptan-3-yl)- 2-(2,6-dioxopiperidin- 3-yl)-4-fluoroisoindoline-1,3-dione |
| | 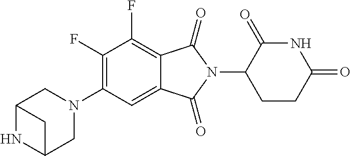 [SMILES: O=C1CCC(N2C(=O)c3cc(N4CC5CC(C4)N5)c(F)c(F)c3C2=O)C(=O)N1] | 6-(3,6-diazabicyclo[3.1.1]heptan-3-yl)- 2-(2,6-dioxopiperidin- 3-yl)-4,5-difluoroisoindoline-1,3-dione |
| | 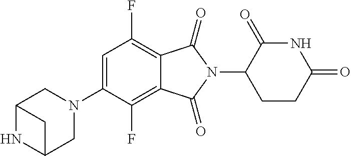 [SMILES: O=C1CCC(N2C(=O)c3c(F)cc(N4CC5CC(C4)N5)c(F)c3C2=O)C(=O)N1] | 5-(3,6-diazabicyclo[3.1.1]heptan-3-yl)- 2-(2,6-dioxopiperidin- 3-yl)-4,7-difluoroisoindoline-1,3-dione |
| | 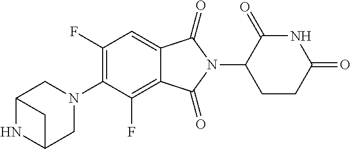 [SMILES: O=C1CCC(N2C(=O)c3cc(F)c(N4CC5CC(C4)N5)c(F)c3C2=O)C(=O)N1] | 5-(3,6-diazabicyclo[3.1.1]heptan-3-yl)- 2-(2,6-dioxopiperidin- 3-yl)-4,6-difluoroisoindoline-1,3-dione |
| | 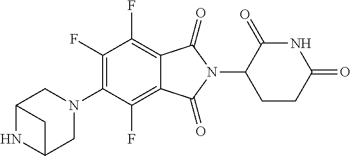 [SMILES: O=C1CCC(N2C(=O)c3c(F)c(F)c(N4CC5CC(C4)N5)c(F)c3C2=O)C(=O)N1] | 5-(3,6-diazabicyclo[3.1.1]heptan-3-yl)- 2-(2,6-dioxopiperidin- 3-yl)-4,6,7-trifluoroisoindoline-1,3-dione |
| | 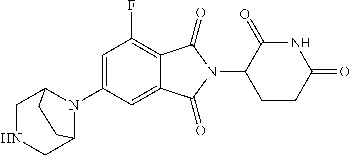 [SMILES: O=C1CCC(N2C(=O)c3cc(N4C5CCC4CNC5)cc(F)c3C2=O)C(=O)N1] | 6-(3,8-diazabicyclo[3.2.1]octan-8-yl)- 2-(2,6-dioxopiperidin-3- yl)-4-fluoroisoindoline-1,3-dione |
| | 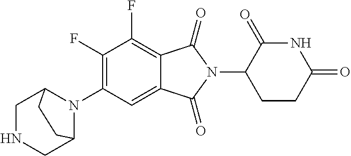 [SMILES: O=C1CCC(N2C(=O)c3cc(N4C5CCC4CNC5)c(F)c(F)c3C2=O)C(=O)N1] | 6-(3,8-diazabicyclo[3.2.1]octan-8-yl)- 2-(2,6-dioxopiperidin-3- yl)-4,5-difluoroisoindoline-1,3-dione |
| | 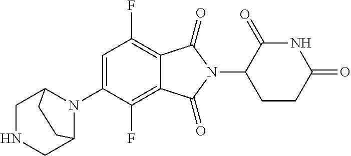 [SMILES: O=C1CCC(N2C(=O)c3c(F)cc(N4C5CCC4CNC5)c(F)c3C2=O)C(=O)N1] | 5-(3,8-diazabicyclo[3.2.1]octan-8-yl)- 2-(2,6-dioxopiperidin-3- yl)-4,7-difluoroisoindoline-1,3-dione |
| | 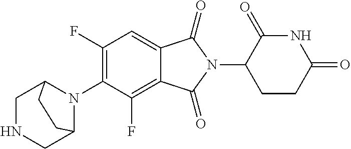 [SMILES: O=C1CCC(N2C(=O)c3cc(F)c(N4C5CCC4CNC5)c(F)c3C2=O)C(=O)N1] | 5-(3,8-diazabicyclo[3.2.1]octan-8-yl)- 2-(2,6-dioxopiperidin- 3-yl)-4,6-difluoroisoindoline-1,3-dione |
| | 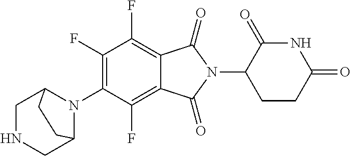 [SMILES: O=C1CCC(N2C(=O)c3c(F)c(F)c(N4C5CCC4CNC5)c(F)c3C2=O)C(=O)N1] | 5-(3,8-diazabicyclo[3.2.1]octan-8-yl)- 2-(2,6-dioxopiperidin- 3-yl)-4,6,7-trifluoroisoindoline-1,3-dione |
| | 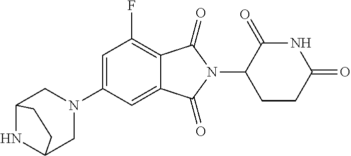 [SMILES: O=C1CCC(N2C(=O)c3cc(N4CC5CCC(C4)N5)cc(F)c3C2=O)C(=O)N1] | 6-(3,8-diazabicyclo[3.2.1]octan-3-yl)- 2-(2,6-dioxopiperidin-3- yl)-4-fluoroisoindoline-1,3-dione |
| | 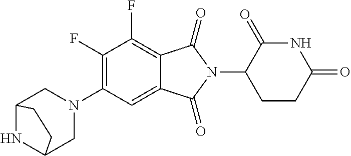 [SMILES: O=C1CCC(N2C(=O)c3cc(N4CC5CCC(C4)N5)c(F)c(F)c3C2=O)C(=O)N1] | 6-(3,8-diazabicyclo[3.2.1]octan-3-yl)- 2-(2,6-dioxopiperidin-3- yl)-4,5-difluoroisoindoline-1,3-dione |
| | 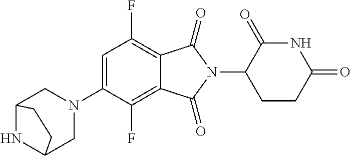 [SMILES: O=C1CCC(N2C(=O)c3c(F)cc(N4CC5CCC(C4)N5)c(F)c3C2=O)C(=O)N1] | 5-(3,8-diazabicyclo[3.2.1]octan-3-yl)- 2-(2,6-dioxopiperidin-3- yl)-4,7-difluoroisoindoline-1,3-dione |
| | 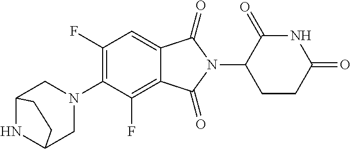 [SMILES: O=C1CCC(N2C(=O)c3cc(F)c(N4CC5CCC(C4)N5)c(F)c3C2=O)C(=O)N1] | 5-(3,8-diazabicyclo[3.2.1]octan-3-yl)- 2-(2,6-dioxopiperidin-3- yl)-4,6-difluoroisoindoline-1,3-dione |
| | 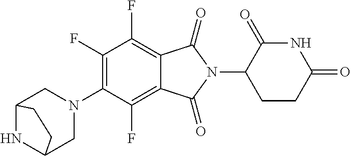 [SMILES: O=C1CCC(N2C(=O)c3c(F)c(F)c(N4CC5CCC(C4)N5)c(F)c3C2=O)C(=O)N1] | 5-(3,8-diazabicyclo[3.2.1]octan-3-yl)- 2-(2,6-dioxopiperidin- 3-yl)-4,6,7-trifluoroisoindoline-1,3-dione |
| | 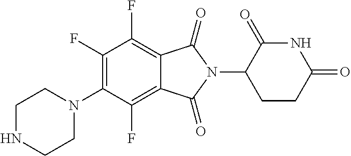 [SMILES: O=C1CCC(N2C(=O)c3c(F)c(F)c(N4CCNCC4)c(F)c3C2=O)C(=O)N1] | 2-(2,6-dioxopiperidin-3-yl)- 4,5,7-trifluoro-6- (piperazin-1-yl)isoindoline- 1,3-dione |
| | 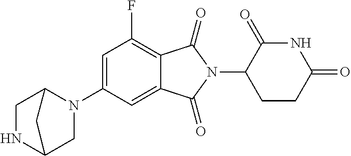 [SMILES: O=C1CCC(N2C(=O)c3cc(N4CC5CC4CN5)cc(F)c3C2=O)C(=O)N1] | 6-(2,5-diazabicyclo[2.2.1]heptan-2-yl)- 2-(2,6-dioxopiperidin-3-yl)- 4-fluoroisoindoline-1,3-dione |
| | 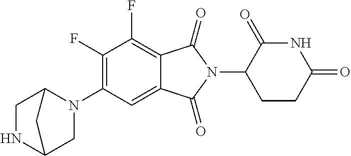 [SMILES: O=C1CCC(N2C(=O)c3cc(N4CC5CC4CN5)c(F)c(F)c3C2=O)C(=O)N1] | 6-(2,5-diazabicyclo[2.2.1]heptan-2-yl)- 2-(2,6-dioxopiperidin-3-yl)- 4,5-difluoroisoindoline-1,3-dione |
| | 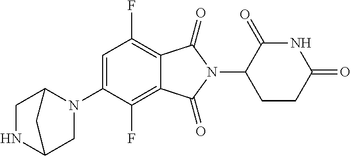 [SMILES: O=C1CCC(N2C(=O)c3c(F)cc(N4CC5CC4CN5)c(F)c3C2=O)C(=O)N1] | 5-(2,5-diazabicyclo[2.2.1]heptan-2-yl)- 2-(2,6-dioxopiperidin-3-yl)- 4,7-difluoroisoindoline-1,3-dione |
| | 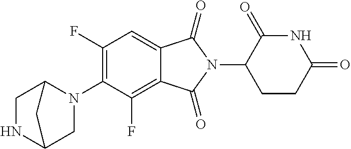 [SMILES: O=C1CCC(N2C(=O)c3cc(F)c(N4CC5CC4CN5)c(F)c3C2=O)C(=O)N1] | 5-(2,5-diazabicyclo[2.2.1]heptan-2-yl)- 2-(2,6-dioxopiperidin-3-yl)- 4,6-difluoroisoindoline-1,3-dione |
| | 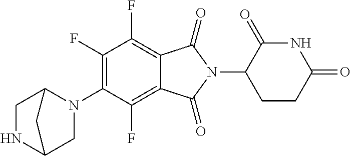 [SMILES: O=C1CCC(N2C(=O)c3c(F)c(F)c(N4CC5CC4CN5)c(F)c3C2=O)C(=O)N1] | 5-(2,5-diazabicyclo[2.2.1]heptan-2-yl)- 2-(2,6-dioxopiperidin-3-yl)- 4,6,7-trifluoroisoindoline-1,3-dione |
| | 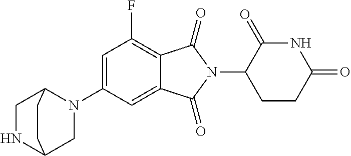 [SMILES: O=C1CCC(N2C(=O)c3cc(N4CC5CCC4CN5)cc(F)c3C2=O)C(=O)N1] | 6-(2,5-diazabicyclo[2.2.1]octan-2-yl)- 2-(2,6-dioxopiperidin-3-yl)- 4-fluoroisoindoline-1,3-dione |
| | 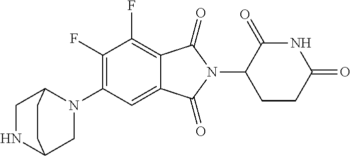 [SMILES: O=C1CCC(N2C(=O)c3cc(N4CC5CCC4CN5)c(F)c(F)c3C2=O)C(=O)N1] | 5-(2,5-diazabicyclo[2.2.1]heptan-2-yl)- 2-(2,6-dioxopiperidin-3-yl)- 4,6-difluoroisoindoline-1,3-dione |
| | 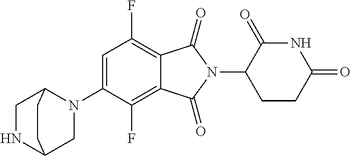 [SMILES: O=C1CCC(N2C(=O)c3c(F)cc(N4CC5CCC4CN5)c(F)c3C2=O)C(=O)N1] | 5-(2,5-diazabicyclo[2.2.1]octan-2-yl)- 2-(2,6-dioxopiperidin-3-yl)- 4,7-difluoroisoindoline-1,3-dione |
| | 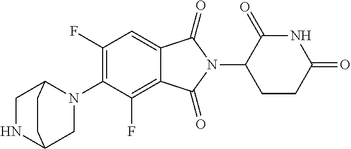 [SMILES: O=C1CCC(N2C(=O)c3cc(F)c(N4CC5CCC4CN5)c(F)c3C2=O)C(=O)N1] | 5-(2,5-diazabicyclo[2.2.1]octan-2-yl)- 2-(2,6-dioxopiperidin-3-yl)- 4,6-difluoroisoindoline-1,3-dione |
| | 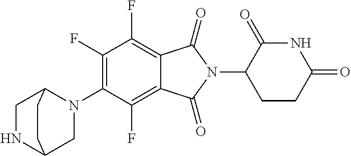 [SMILES: O=C1CCC(N2C(=O)c3c(F)c(F)c(N4CC5CCC4CN5)c(F)c3C2=O)C(=O)N1] | 5-(2,5-diazabicyclo[2.2.1]octan-2-yl)- 2-(2,6-dioxopiperidin-3-yl)- 4,6,7-trifluoroisoindoline-1,3-dione |
| | 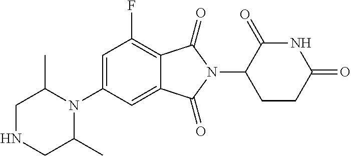 [SMILES: CC1CNCC(C)N1c1cc(F)c2c(c1)C(=O)N(C1CCC(=O)NC1=O)C2=O] | 6-(2,6-dimethylpiperazin-1-yl)- 2-(2,6-dioxopiperidin-3-yl)- 4-fluoroisoindoline-1,3-dione |
| | 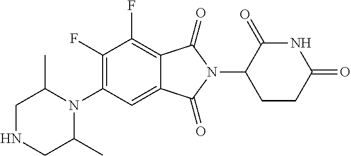 [SMILES: CC1CNCC(C)N1c1cc2c(c(F)c1F)C(=O)N(C1CCC(=O)NC1=O)C2=O] | 6-(2,6-dimethylpiperazin-1-yl)- 2-(2,6-dioxopiperidin-3-yl)- 4,5-difluoroisoindoline-1,3-dione |
| | 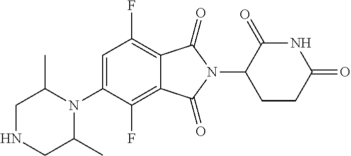 [SMILES: CC1CNCC(C)N1c1cc(F)c2c(c1F)C(=O)N(C1CCC(=O)NC1=O)C2=O] | 5-(2,6-dimethylpiperazin-1-yl)- 2-(2,6-dioxopiperidin-3-yl)- 4,7-difluoroisoindoline-1,3-dione |
| | 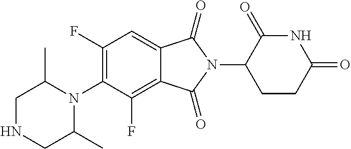 [SMILES: CC1CNCC(C)N1c1c(F)cc2c(c1F)C(=O)N(C1CCC(=O)NC1=O)C2=O] | 5-(2,6-dimethylpiperazin-1-yl)- 2-(2,6-dioxopiperidin-3-yl)- 4,6-difluoroisoindoline-1,3-dione |
| | 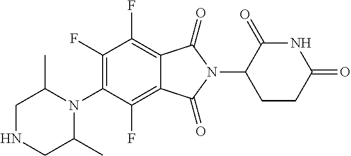 [SMILES: CC1CNCC(C)N1c1c(F)c(F)c2c(c1F)C(=O)N(C1CCC(=O)NC1=O)C2=O] | 5-(2,6-dimethylpiperazin-1-yl)- 2-(2,6-dioxopiperidin-3-yl)- 4,6,7-trifluoroisoindoline-1,3-dione |
| | 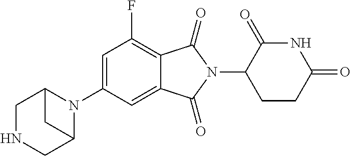 [SMILES: O=C1CCC(N2C(=O)c3cc(N4C5CNCC4C5)cc(F)c3C2=O)C(=O)N1] | 6-(3,6-diazabicyclo[3.1.1]heptan-6-yl)- 2-(2,6-dioxopiperidin-3-yl)- 4-fluoroisoindoline-1,3-dione |
| | 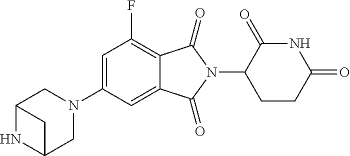 [SMILES: O=C1CCC(N2C(=O)c3cc(N4CC5CC(C4)N5)cc(F)c3C2=O)C(=O)N1] | 6-(3,6-diazabicyclo[3.1.1]heptan-3-yl)- 2-(2,6-dioxopiperidin-3-yl)- 4-fluoroisoindoline-1,3-dione |
| | 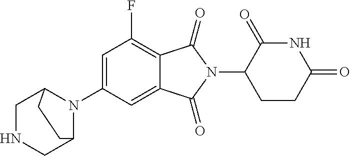 [SMILES: O=C1CCC(N2C(=O)c3cc(N4C5CCC4CNC5)cc(F)c3C2=O)C(=O)N1] | 6-(3,8-diazabicyclo[3.3.1]hoctan-8-yl)- 2-(2,6-dioxopiperidin-3-yl)- 4-fluoroisoindoline-1,3-dione |
| | 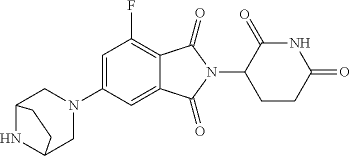 [SMILES: O=C1CCC(N2C(=O)c3cc(N4CC5CCC(C4)N5)cc(F)c3C2=O)C(=O)N1] | 6-(3,8-diazabicyclo[3.2.1]octan-3-yl)- 2-(2,6-dioxopiperidin-3- yl)-4-fluoroisoindoline-1,3-dione |
| | 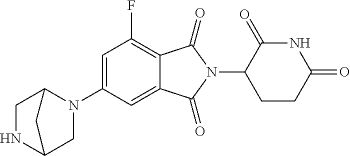 [SMILES: O=C1CCC(N2C(=O)c3cc(N4CC5CC4CN5)cc(F)c3C2=O)C(=O)N1] | 6-(2,5-diazabicyclo[2.2.1]heptan-2-yl)- 2-(2,6-dioxopiperidin- 3-yl)-4-fluoroisoindoline-1,3-dione |
| | 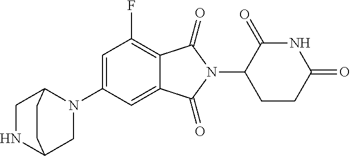 [SMILES: O=C1CCC(N2C(=O)c3cc(N4CC5CCC4CN5)cc(F)c3C2=O)C(=O)N1] | 6-(2,5-diazabicyclo[2.2.2]octan-2-yl)- 2-(2,6-dioxopiperidin-3- yl)-4-fluoroisoindoline-1,3-dione |
| | 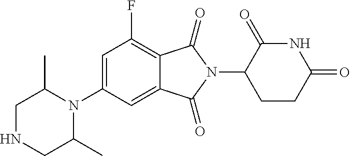 [SMILES: CC1CNCC(C)N1c1cc(F)c2c(c1)C(=O)N(C1CCC(=O)NC1=O)C2=O] | 6-(2,6-dimethylpiperazin-1-yl)- 2-(2,6-dioxopiperidin-3-yl)-4- fluoroisoindoline-1,3-dione |
| | 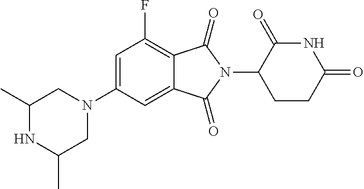 [SMILES: CC1CN(c2cc(F)c3c(c2)C(=O)N(C2CCC(=O)NC2=O)C3=O)CC(C)N1] | 6-(3,5-dimethylpiperazin-1-yl)- 2-(2,6-dioxopiperidin-3-yl)-4- fluoroisoindoline-1,3-dione |
| | 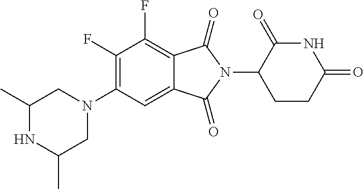 [SMILES: CC1CN(c2cc3c(c(F)c2F)C(=O)N(C2CCC(=O)NC2=O)C3=O)CC(C)N1] | 6-(3,5-dimethylpiperazin-1-yl)- 2-(2,6-dioxopiperidin-3-yl)- 4,5-difluoroisoindoline-1,3-dione |
| | 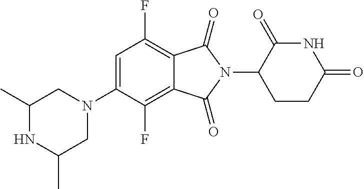 [SMILES: CC1CN(c2cc(F)c3c(c2F)C(=O)N(C2CCC(=O)NC2=O)C3=O)CC(C)N1] | 5-(3,5-dimethylpiperazin-1-yl)- 2-(2,6-dioxopiperidin-3-yl)- 4,7-difluoroisoindoline-1,3-dione |
| | 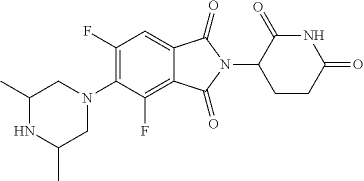 [SMILES: CC1CN(c2c(F)cc3c(c2F)C(=O)N(C2CCC(=O)NC2=O)C3=O)CC(C)N1] | 5-(3,5-dimethylpiperazin-1-yl)- 2-(2,6-dioxopiperidin-3-yl)- 4,6-difluoroisoindoline-1,3-dione |
| | 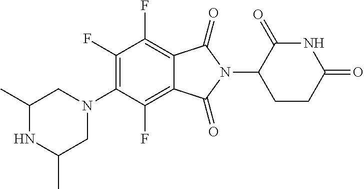 [SMILES: CC1CN(c2c(F)c(F)c3c(c2F)C(=O)N(C2CCC(=O)NC2=O)C3=O)CC(C)N1] | 5-(3,5-dimethylpiperazin-1-yl)- 2-(2,6-dioxopiperidin-3-yl)- 4,6,7-trifluoroisoindoline-1,3-dione |
| | 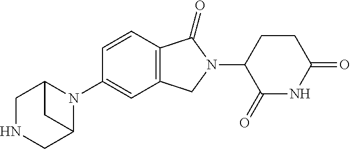 [SMILES: O=C1CCC(N2Cc3cc(N4C5CNCC4C5)ccc3C2=O)C(=O)N1] | 3-(5-(3,6-diazabicyclo[3.1.1]heptan-6-yl)- 1-oxoisoindolin-2- yl)piperidine-2,6-dione |
| | 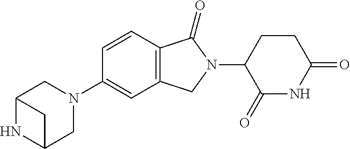 [SMILES: O=C1CCC(N2Cc3cc(N4CC5CC(C4)N5)ccc3C2=O)C(=O)N1] | 3-(5-(3,6-diazabicyclo[3.1.1]heptan-3-yl)- 1-oxoisoindolin-2- yl)piperidine-2,6-dione |
| | 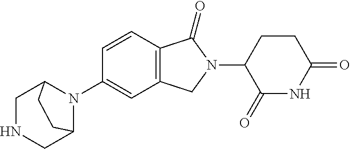 [SMILES: O=C1CCC(N2Cc3cc(N4C5CCC4CNC5)ccc3C2=O)C(=O)N1] | 3-(5-(3,8-diazabicyclo[3.2.1]octan-8-yl)- 1-oxoisoindolin-2- yl)piperidine-2,6-dione |
| | 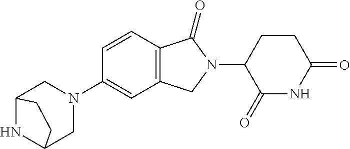 [SMILES: O=C1CCC(N2Cc3cc(N4CC5CCC(C4)N5)ccc3C2=O)C(=O)N1] | 3-(5-(3,8-diazabicyclo[3.2.1]octan-3-yl)- 1-oxoisoindolin-2- yl)piperidine-2,6-dione |
| | 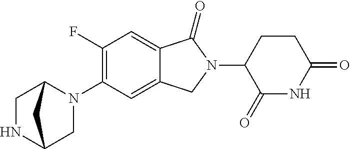 [SMILES: O=C1CCC(N2Cc3cc(N4C[C@H]5C[C@@H]4CN5)c(F)cc3C2=O)C(=O)N1] | 3-(5-((1R,4R)-2,5-diazabicyclo[2.2.1]heptan-2-yl)- 6-fluoro-1-oxoisoindolin-2-yl)piperidine-2,6-dione |
| | 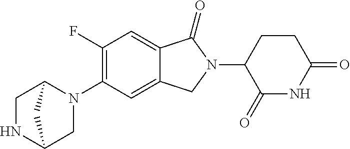 [SMILES: O=C1CCC(N2Cc3cc(N4C[C@@H]5C[C@H]4CN5)c(F)cc3C2=O)C(=O)N1] | 3-(5-((1S,4S)-2,5-diazabicyclo[2.2.1]heptan-2-yl)- 6-fluoro-1-oxoisoindolin-2-yl)piperidine-2,6-dione |
| | 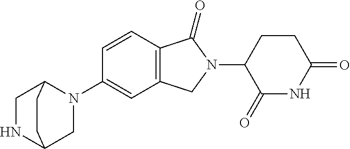 [SMILES: O=C1CCC(N2Cc3cc(N4CC5CCC4CN5)ccc3C2=O)C(=O)N1] | 3-(5-(2,5-diazabicyclo[2.2.2]octan-2-yl)- 1-oxoisoindolin-2- yl)piperidine-2,6-dione |
| | 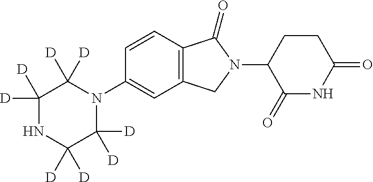 [SMILES: [2H]C1([2H])NC([2H])([2H])C([2H])([2H])N(c2ccc3c(c2)CN(C2CCC(=O)NC2=O)C3=O)C1([2H])[2H]] | 3-(1-oxo-5- (piperazin-1-yl-2,2,3,3,5,5,6,6-d₈)isoindolin-2- yl)piperidine-2,6-dione |
| | 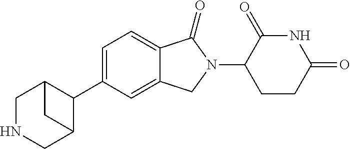 [SMILES: O=C1CCC(N2Cc3cc(C4C5CNCC4C5)ccc3C2=O)C(=O)N1] | 3-(5-(3-azabicyclo[3.1.1]heptan-6-yl)- 1-oxoisoindolin-2- yl)piperidine-2,6-dione |
| | 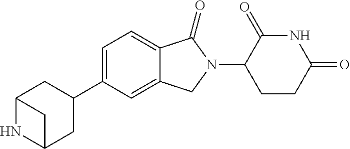 [SMILES: O=C1CCC(N2Cc3cc(C4CC5CC(C4)N5)ccc3C2=O)C(=O)N1] | 3-(5-(6-azabicyclo[3.1.1]heptan-3-yl)- 1-oxoisoindolin-2- yl)piperidine-2,6-dione |
| | 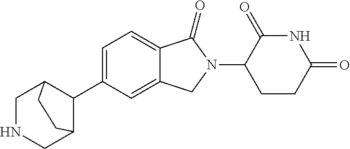 [SMILES: O=C1CCC(N2Cc3cc(C4C5CCC4CNC5)ccc3C2=O)C(=O)N1] | 3-(5-(3-azabicyclo[3.2.1]octan-8-yl)- 1-oxoisoindolin-2- yl)piperidine-2,6-dione |
| | 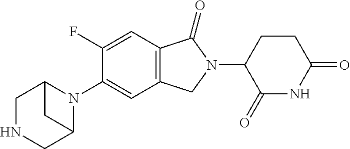 [SMILES: O=C1CCC(N2Cc3cc(N4C5CNCC4C5)c(F)cc3C2=O)C(=O)N1] | 3-(5-(3,6-diazabicyclo[3.1.1]heptan-6-yl)- 6-fluoro-1- oxoisoindolin-2-yl)piperidine-2,6-dione |
| | 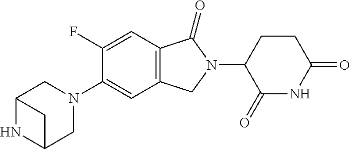 [SMILES: O=C1CCC(N2Cc3cc(N4CC5CC(C4)N5)c(F)cc3C2=O)C(=O)N1] | 3-(5-(3,6-diazabicyclo[3.1.1]heptan-3-yl)- 6-fluoro-1- oxoisoindolin-2-yl)piperidine-2,6-dione |
| | 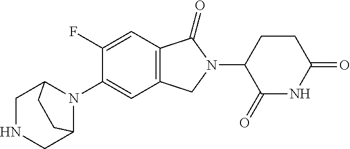 [SMILES: O=C1CCC(N2Cc3cc(N4C5CCC4CNC5)c(F)cc3C2=O)C(=O)N1] | 3-(5-(3,8-diazabicyclo[3.2.1]octan-8-yl)- 6-fluoro-1- oxoisoindolin-2-yl)piperidine-2,6-dione |
| | 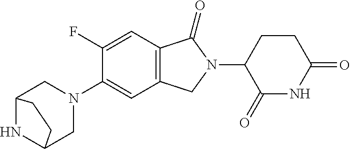 [SMILES: O=C1CCC(N2Cc3cc(N4CC5CCC(C4)N5)c(F)cc3C2=O)C(=O)N1] | 3-(5-(3,8-diazabicyclo[3.2.1]octan-3-yl)- 6-fluoro-1- oxoisoindolin-2-yl)piperidine-2,6-dione |
| | 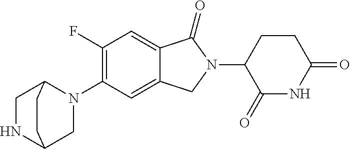 [SMILES: O=C1CCC(N2Cc3cc(N4CC5CCC4CN5)c(F)cc3C2=O)C(=O)N1] | 3-(5-(2,5-diazabicyclo[2.2.2]octan-2-yl)- 6-fluoro-1- oxoisoindolin-2-yl)piperidine-2,6-dione |
| | 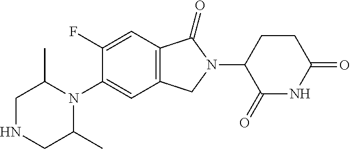 [SMILES: CC1CNCC(C)N1c1cc2c(cc1F)C(=O)N(C1CCC(=O)NC1=O)C2] | 3-(5-(2,6-dimethylpiperazin-1-yl)- 6-fluoro-1-oxoisoindolin-2- yl)piperidine-2,6-dione |
| | 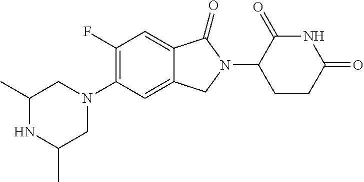 [SMILES: CC1CN(c2cc3c(cc2F)C(=O)N(C2CCC(=O)NC2=O)C3)CC(C)N1] | 3-(5-(3,5-dimethylpiperazin-1-yl)- 6-fluoro-1-oxoisoindolin-2- yl)piperidine-2,6-dione |
| | 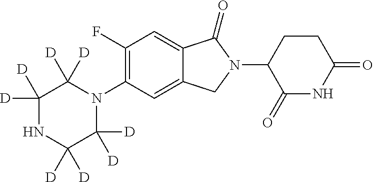 [SMILES: [2H]C1([2H])NC([2H])([2H])C([2H])([2H])N(c2cc3c(cc2F)C(=O)N(C2CCC(=O)NC2=O)C3)C1([2H])[2H]] | 3-(6-fluoro-1-oxo-5- (piperazin-1-yl-2,2,3,3,5,5,6,6- d₈)isoindolin-2-yl)piperidine-2,6-dione |
| | 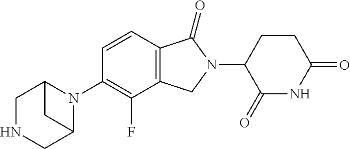 [SMILES: O=C1CCC(N2Cc3c(ccc(N4C5CNCC4C5)c3F)C2=O)C(=O)N1] | 3-(5-(3,6-diazabicyclo[3.1.1]heptan-6-yl)- 4-fluoro-1- oxoisoindolin-2-yl)piperidine-2,6-dione |
| | 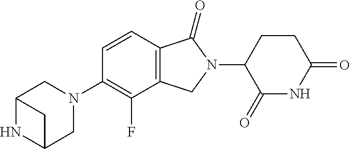 [SMILES: O=C1CCC(N2Cc3c(ccc(N4CC5CC(C4)N5)c3F)C2=O)C(=O)N1] | 3-(5-(3,6-diazabicyclo[3.1.1]heptan-3-yl)- 4-fluoro-1- oxoisoindolin-2-yl)piperidine-2,6-dione |
| | 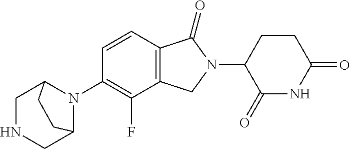 [SMILES: O=C1CCC(N2Cc3c(ccc(N4C5CCC4CNC5)c3F)C2=O)C(=O)N1] | 3-(5-(3,8-diazabicyclo[3.2.1]octan-8-yl)- 4-fluoro-1- oxoisoindolin-2-yl)piperidine-2,6-dione |
| | 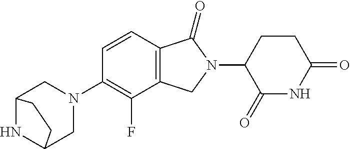 [SMILES: O=C1CCC(N2Cc3c(ccc(N4CC5CCC(C4)N5)c3F)C2=O)C(=O)N1] | 3-(5-(3,8-diazabicyclo[3.2.1]octan-3-yl)- 4-fluoro-1- oxoisoindolin-2-yl)piperidine-2,6-dione |
| | 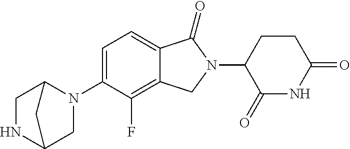 [SMILES: O=C1CCC(N2Cc3c(ccc(N4CC5CC4CN5)c3F)C2=O)C(=O)N1] | 3-(5-(2,5-diazabicyclo[2.2.1]heptan-2-yl)- 4-fluoro-1- oxoisoindolin-2-yl)piperidine-2,6-dione |
| | 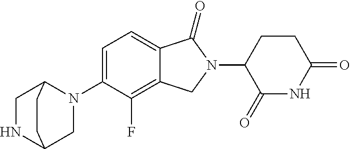 [SMILES: O=C1CCC(N2Cc3c(ccc(N4CC5CCC4CN5)c3F)C2=O)C(=O)N1] | 3-(5-(2,5-diazabicyclo[2.2.2]octan-2-yl)- 4-fluoro-1- oxoisoindolin-2-yl)piperidine-2,6-dione |
| | 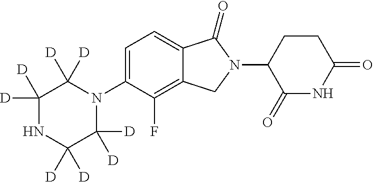 [SMILES: [2H]C1([2H])NC([2H])([2H])C([2H])([2H])N(c2ccc3c(c2F)CN(C2CCC(=O)NC2=O)C3=O)C1([2H])[2H]] | 3-(4-fluoro-1-oxo-5- (piperazin-1-yl-2,2,3,3,5,5,6,6- d₈)isoindolin-2-yl)piperidine-2,6-dione |
| | 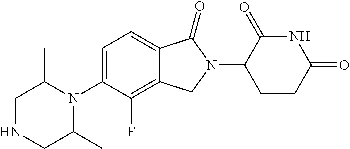 [SMILES: CC1CNCC(C)N1c1ccc2c(c1F)CN(C1CCC(=O)NC1=O)C2=O] | 3-(5-(2,6-dimethylpiperazin-1-yl)- 4-fluoro-1-oxoisoindolin-2- yl)piperidine-2,6-dione |
| | 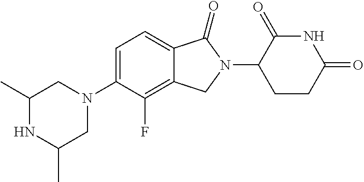 [SMILES: CC1CN(c2ccc3c(c2F)CN(C2CCC(=O)NC2=O)C3=O)CC(C)N1] | 3-(5-(3,5-dimethylpiperazin-1-yl)- 4-fluoro-1-oxoisoindolin-2- yl)piperidine-2,6-dione |
| | 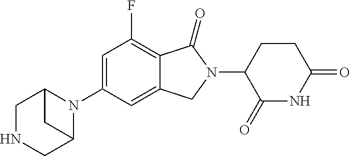 [SMILES: O=C1CCC(N2Cc3cc(N4C5CNCC4C5)cc(F)c3C2=O)C(=O)N1] | 3-(5-(3,6-diazabicyclo[3.1.1]heptan-6-yl)- 7-fluoro-1- oxoisoindolin-2-yl)piperidine-2,6-dione |
| | 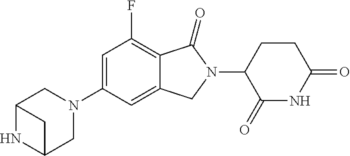 [SMILES: O=C1CCC(N2Cc3cc(N4CC5CC(C4)N5)cc(F)c3C2=O)C(=O)N1] | 3-(5-(3,6-diazabicyclo[3.1.1]heptan-3-yl)- 7-fluoro-1- oxoisoindolin-2-yl)piperidine-2,6-dione |
| | 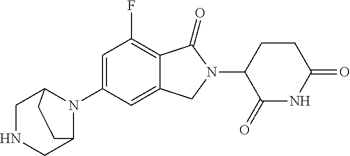 [SMILES: O=C1CCC(N2Cc3cc(N4C5CCC4CNC5)cc(F)c3C2=O)C(=O)N1] | 3-(5-(3,8-diazabicyclo[3.2.1]octan-8-yl)- 7-fluoro-1- oxoisoindolin-2-yl)piperidine-2,6-dione |
| | 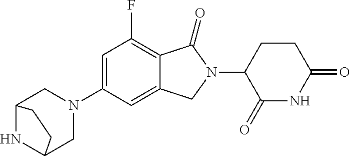 [SMILES: O=C1CCC(N2Cc3cc(N4CC5CCC(C4)N5)cc(F)c3C2=O)C(=O)N1] | 3-(5-(3,8-diazabicyclo[3.2.1]octan-3-yl)- 7-fluoro-1- oxoisoindolin-2-yl)piperidine-2,6-dione |
| | 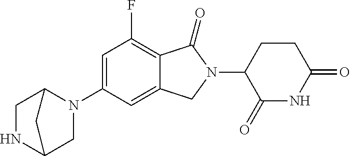 [SMILES: O=C1CCC(N2Cc3cc(N4CC5CC4CN5)cc(F)c3C2=O)C(=O)N1] | 3-(5-(2,5-diazabicyclo[2.2.1]heptan-2-yl)- 7-fluoro-1- oxoisoindolin-2-yl)piperidine-2,6-dione |
| | 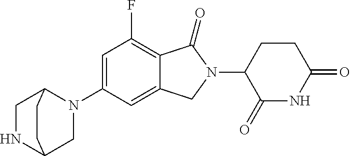 [SMILES: O=C1CCC(N2Cc3cc(N4CC5CCC4CN5)cc(F)c3C2=O)C(=O)N1] | 3-(5-(2,5-diazabicyclo[2.2.2]octan-2-yl)- 7-fluoro-1- oxoisoindolin-2-yl)piperidine-2,6-dione |
| | 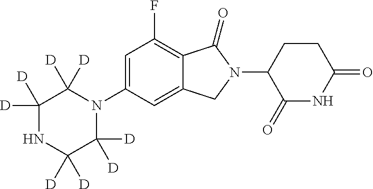 [SMILES: [2H]C1([2H])NC([2H])([2H])C([2H])([2H])N(c2cc(F)c3c(c2)CN(C2CCC(=O)NC2=O)C3=O)C1([2H])[2H]] | 3-(7-fluoro-1-oxo-5- (piperazin-1-yl-2,2,3,3,5,5,6,6- d₈)isoindolin-2-yl)piperidine-2,6-dione |
| | 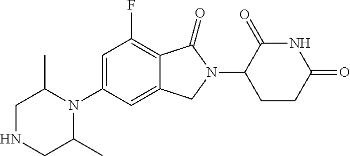 [SMILES: CC1CNCC(C)N1c1cc(F)c2c(c1)CN(C1CCC(=O)NC1=O)C2=O] | 3-(5-(2,6-dimethylpiperazin-1-yl)- 7-fluoro-1-oxoisoindolin-2- yl)piperidine-2,6-dione |
| | 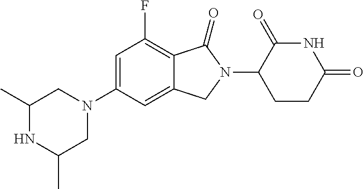 [SMILES: CC1CN(c2cc(F)c3c(c2)CN(C2CCC(=O)NC2=O)C3=O)CC(C)N1] | 3-(5-(3,5-dimethylpiperazin-1-yl)- 7-fluoro-1-oxoisoindolin-2- yl)piperidine-2,6-dione |
| | 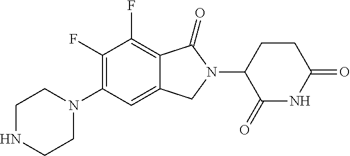 [SMILES: O=C1CCC(N2Cc3cc(N4CCNCC4)c(F)c(F)c3C2=O)C(=O)N1] | 3-(6,7-difluoro-1-oxo-5- (piperazin-1-yl)isoindolin-2- yl)piperidine-2,6-dione |
| | 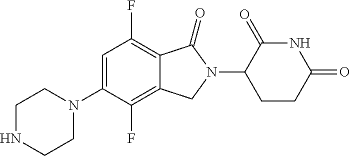 [SMILES: O=C1CCC(N2Cc3c(F)c(N4CCNCC4)cc(F)c3C2=O)C(=O)N1] | 3-(4,7-difluoro-1-oxo-5- (piperazin-1-yl)isoindolin-2- yl)piperidine-2,6-dione |
| | 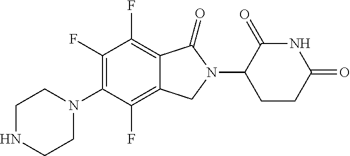 [SMILES: O=C1CCC(N2Cc3c(F)c(N4CCNCC4)c(F)c(F)c3C2=O)C(=O)N1] | 3-(4,6,7-trifluoro-1-oxo-5- (piperazin-1-yl)isoindolin-2- yl)piperidine-2,6-dione |
| | 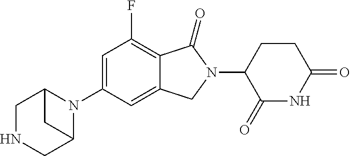 [SMILES: O=C1CCC(N2Cc3cc(N4C5CNCC4C5)cc(F)c3C2=O)C(=O)N1] | 3-(5-(3,6-diazabicyclo[3.1.1]heptan-6-yl)- 7-fluoro-1- oxoisoindolin-2-yl)piperidine-2,6-dione |
| | 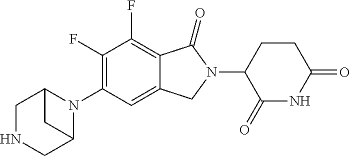 [SMILES: O=C1CCC(N2Cc3cc(N4C5CNCC4C5)c(F)c(F)c3C2=O)C(=O)N1] | 3-(5-(3,6-diazabicyclo[3.1.1]heptan-6-yl)- 6,7-difluoro-1- oxoisoindolin-2-yl)piperidine-2,6-dione |
| | 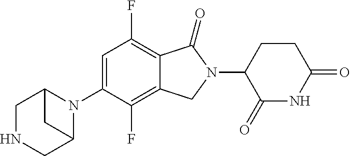 [SMILES: O=C1CCC(N2Cc3c(F)c(N4C5CNCC4C5)cc(F)c3C2=O)C(=O)N1] | 3-(5-(3,6-diazabicyclo[3.1.1]heptan-6-yl)- 4,7-difluoro-1- oxoisoindolin-2-yl)piperidine-2,6-dione |
| | 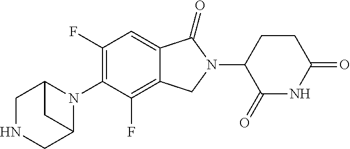 [SMILES: O=C1CCC(N2Cc3c(cc(F)c(N4C5CNCC4C5)c3F)C2=O)C(=O)N1] | 3-(5-(3,6-diazabicyclo[3.1.1]heptan-6-yl)- 4,6-difluoro-1- oxoisoindolin-2-yl)piperidine-2,6-dione |
| | 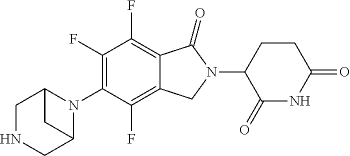 [SMILES: O=C1CCC(N2Cc3c(F)c(N4C5CNCC4C5)c(F)c(F)c3C2=O)C(=O)N1] | 3-(5-(3,6-diazabicyclo[3.1.1]heptan-6-yl)- 4,6,7-trifluoro-1- oxoisoindolin-2-yl)piperidine-2,6-dione |
| | 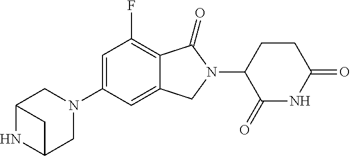 [SMILES: O=C1CCC(N2Cc3cc(N4CC5CC(C4)N5)cc(F)c3C2=O)C(=O)N1] | 3-(5-(3,6-diazabicyclo[3.1.1]heptan-3-yl)- 7-fluoro-1- oxoisoindolin-2-yl)piperidine-2,6-dione |
| | 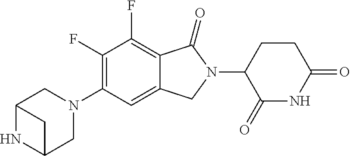 [SMILES: O=C1CCC(N2Cc3cc(N4CC5CC(C4)N5)c(F)c(F)c3C2=O)C(=O)N1] | 3-(5-(3,6-diazabicyclo[3.1.1]heptan-3-yl)- 6,7-difluoro-1- oxoisoindolin-2-yl)piperidine-2,6-dione |
| | 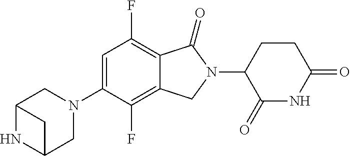 [SMILES: O=C1CCC(N2Cc3c(F)c(N4CC5CC(C4)N5)cc(F)c3C2=O)C(=O)N1] | 3-(5-(3,6-diazabicyclo[3.1.1]heptan-3-yl)- 4,7-difluoro-1- oxoisoindolin-2-yl)piperidine-2,6-dione |
| | 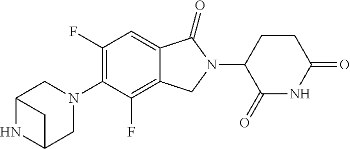 [SMILES: O=C1CCC(N2Cc3c(cc(F)c(N4CC5CC(C4)N5)c3F)C2=O)C(=O)N1] | 3-(5-(3,6-diazabicyclo[3.1.1]heptan-3-yl)- 4,6-difluoro-1- oxoisoindolin-2-yl)piperidine-2,6-dione |
| | 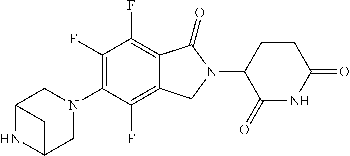 [SMILES: O=C1CCC(N2Cc3c(F)c(N4CC5CC(C4)N5)c(F)c(F)c3C2=O)C(=O)N1] | 3-(5-(3,6-diazabicyclo[3.1.1]heptan-3-yl)- 4,6,7-trifluoro-1- oxoisoindolin-2-yl)piperidine-2,6-dione |
| | 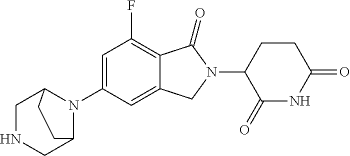 [SMILES: O=C1CCC(N2Cc3cc(N4C5CCC4CNC5)cc(F)c3C2=O)C(=O)N1] | 3-(5-(3,8-diazabicyclo[3.2.1]octan-8-yl)- 7-fluoro-1- oxoisoindolin-2-yl)piperidine-2,6-dione |
| | 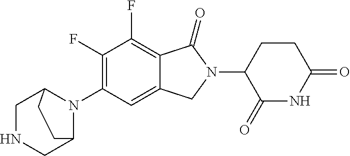 [SMILES: O=C1CCC(N2Cc3cc(N4C5CCC4CNC5)c(F)c(F)c3C2=O)C(=O)N1] | 3-(5-(3,8-diazabicyclo[3.2.1]octan-8-yl)- 6,7-difluoro-1- oxoisoindolin-2-yl)piperidine-2,6-dione |
| | 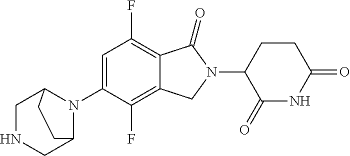 [SMILES: O=C1CCC(N2Cc3c(F)c(N4C5CCC4CNC5)cc(F)c3C2=O)C(=O)N1] | 3-(5-(3,8-diazabicyclo[3.2.1]octan-8-yl)- 4,7-difluoro-1- oxoisoindolin-2-yl)piperidine-2,6-dione |
| | 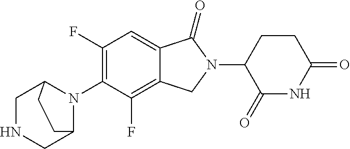 [SMILES: O=C1CCC(N2Cc3c(cc(F)c(N4C5CCC4CNC5)c3F)C2=O)C(=O)N1] | 3-(5-(3,8-diazabicyclo[3.2.1]octan-8-yl)- 4,6-difluoro-1- oxoisoindolin-2-yl)piperidine-2,6-dione |
| | 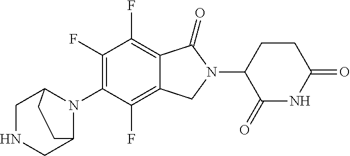 [SMILES: O=C1CCC(N2Cc3c(F)c(N4C5CCC4CNC5)c(F)c(F)c3C2=O)C(=O)N1] | 3-(5-(3,8-diazabicyclo[3.2.1]octan-8-yl)- 4,6,7-trifluoro-1- oxoisoindolin-2-yl)piperidine-2,6-dione |
| | 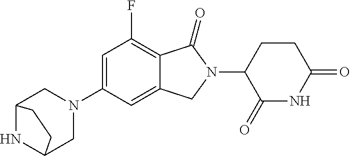 [SMILES: O=C1CCC(N2Cc3cc(N4CC5CCC(C4)N5)cc(F)c3C2=O)C(=O)N1] | 3-(5-(3,8-diazabicyclo[3.2.1]octan-3-yl)- 7-fluoro-1- oxoisoindolin-2-yl)piperidine-2,6-dione |
| | 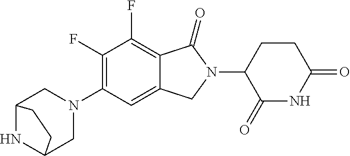 [SMILES: O=C1CCC(N2Cc3cc(N4CC5CCC(C4)N5)c(F)c(F)c3C2=O)C(=O)N1] | 3-(5-(3,8-diazabicyclo[3.2.1]octan-3-yl)- 6,7-difluoro-1- oxoisoindolin-2-yl)piperidine-2,6-dione |
| | 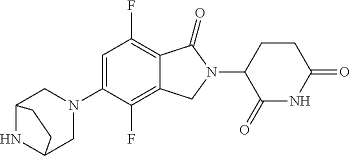 [SMILES: O=C1CCC(N2Cc3c(F)c(N4CC5CCC(C4)N5)cc(F)c3C2=O)C(=O)N1] | 3-(5-(3,8-diazabicyclo[3.2.1]octan-3-yl)- 4,7-difluoro-1- oxoisoindolin-2-yl)piperidine-2,6-dione |
| | 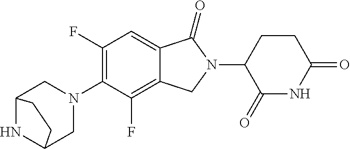 [SMILES: O=C1CCC(N2Cc3c(cc(F)c(N4CC5CCC(C4)N5)c3F)C2=O)C(=O)N1] | 3-(5-(3,8-diazabicyclo[3.2.1]octan-3-yl)- 4,6-difluoro-1- oxoisoindolin-2-yl)piperidine-2,6-dione |
| | 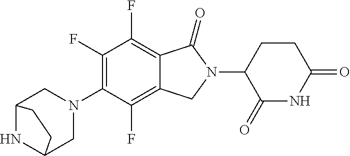 [SMILES: O=C1CCC(N2Cc3c(F)c(N4CC5CCC(C4)N5)c(F)c(F)c3C2=O)C(=O)N1] | 3-(5-(3,8-diazabicyclo[3.2.1]octan-3-yl)- 4,6,7-trifluoro-1- oxoisoindolin-2-yl)piperidine-2,6-dione |
| | 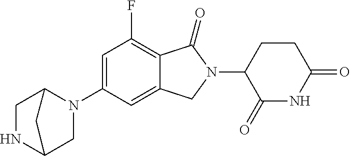 [SMILES: O=C1CCC(N2Cc3cc(N4CC5CC4CN5)cc(F)c3C2=O)C(=O)N1] | 3-(5-(2,5-diazabicyclo[2.2.1]heptan-2-yl)- 7-fluoro-1- oxoisoindolin-2-yl)piperidine-2,6-dione |
| | 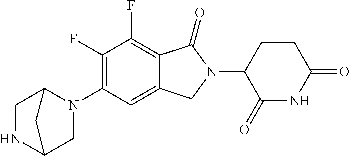 [SMILES: O=C1CCC(N2Cc3cc(N4CC5CC4CN5)c(F)c(F)c3C2=O)C(=O)N1] | 3-(5-(2,5-diazabicyclo[2.2.1]heptan-2-yl)- 6,7-difluoro-1- oxoisoindolin-2-yl)piperidine-2,6-dione |
| | 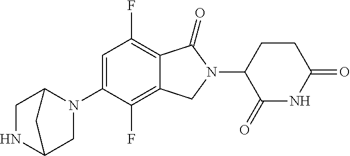 [SMILES: O=C1CCC(N2Cc3c(F)c(N4CC5CC4CN5)cc(F)c3C2=O)C(=O)N1] | 3-(5-(2,5-diazabicyclo[2.2.1]heptan-2-yl)- 4,7-difluoro-1- oxoisoindolin-2-yl)piperidine-2,6-dione |
| | 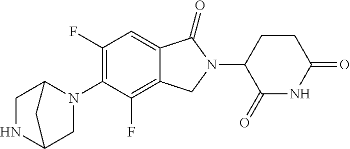 [SMILES: O=C1CCC(N2Cc3c(cc(F)c(N4CC5CC4CN5)c3F)C2=O)C(=O)N1] | 3-(5-(2,5-diazabicyclo[2.2.1]heptan-2-yl)- 4,6-difluoro-1- oxoisoindolin-2-yl)piperidine-2,6-dione |
| | 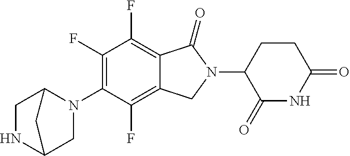 [SMILES: O=C1CCC(N2Cc3c(F)c(N4CC5CC4CN5)c(F)c(F)c3C2=O)C(=O)N1] | 3-(5-(2,5-diazabicyclo[2.2.1]heptan-2-yl)- 4,6,7-trifluoro-1- oxoisoindolin-2-yl)piperidine-2,6-dione |
| | 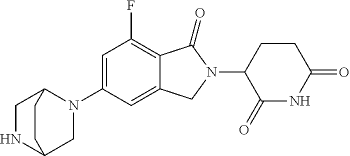 [SMILES: O=C1CCC(N2Cc3cc(N4CC5CCC4CN5)cc(F)c3C2=O)C(=O)N1] | 3-(5-(2,5-diazabicyclo[2.2.2]octan-2-yl)- 7-fluoro-1- oxoisoindolin-2-yl)piperidine-2,6-dione |
| | 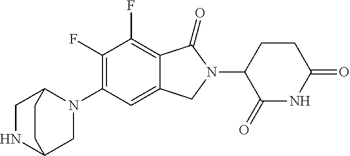 [SMILES: O=C1CCC(N2Cc3cc(N4CC5CCC4CN5)c(F)c(F)c3C2=O)C(=O)N1] | 3-(5-(2,5-diazabicyclo[2.2.2]octan-2-yl)- 6,7-difluoro-1- oxoisoindolin-2-yl)piperidine-2,6-dione |
| | 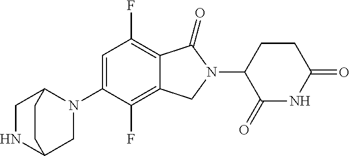 [SMILES: O=C1CCC(N2Cc3c(F)c(N4CC5CCC4CN5)cc(F)c3C2=O)C(=O)N1] | 3-(5-(2,5-diazabicyclo[2.2.2]octan-2-yl)- 4,7-difluoro-1- oxoisoindolin-2-yl)piperidine-2,6-dione |
| | 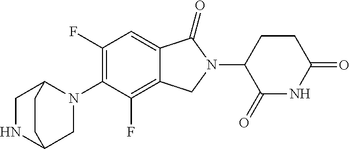 [SMILES: O=C1CCC(N2Cc3c(cc(F)c(N4CC5CCC4CN5)c3F)C2=O)C(=O)N1] | 3-(5-(2,5-diazabicyclo[2.2.2]octan-2-yl)- 4,6-difluoro-1- oxoisoindolin-2-yl)piperidine-2,6-dione |
| | 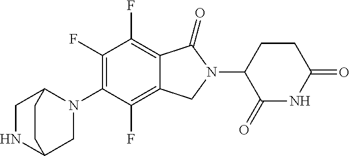 [SMILES: O=C1CCC(N2Cc3c(F)c(N4CC5CCC4CN5)c(F)c(F)c3C2=O)C(=O)N1] | 3-(5-(2,5-diazabicyclo[2.2.2]octan-2-yl)- 4,6,7-trifluoro-1- oxoisoindolin-2-yl)piperidine-2,6-dione |
| | 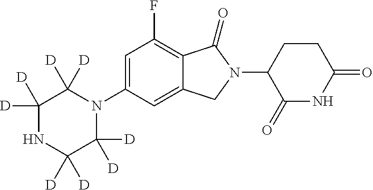 [SMILES: [2H]C1([2H])NC([2H])([2H])C([2H])([2H])N(c2cc(F)c3c(c2)CN(C2CCC(=O)NC2=O)C3=O)C1([2H])[2H]] | 3-(7-fluoro-1-oxo-5- (piperazin-1-yl-2,2,3,3,5,5,6,6- d₈)isoindolin-2-yl)piperidine-2,6-dione |
| | 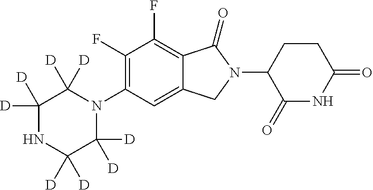 [SMILES: [2H]C1([2H])NC([2H])([2H])C([2H])([2H])N(c2cc3c(c(F)c2F)C(=O)N(C2CCC(=O)NC2=O)C3)C1([2H])[2H]] | 3-(6,7-difluoro-1-oxo-5- (piperazin-1-yl-2,2,3,3,5,5,6,6- d₈)isoindolin-2-yl)piperidine-2,6-dione |
| | 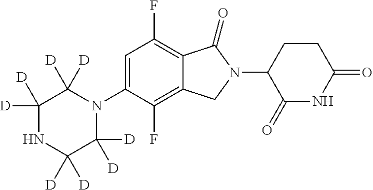 [SMILES: [2H]C1([2H])NC([2H])([2H])C([2H])([2H])N(c2cc(F)c3c(c2F)CN(C2CCC(=O)NC2=O)C3=O)C1([2H])[2H]] | 3-(4,7-difluoro-1-oxo-5- (piperazin-1-yl-2,2,3,3,5,5,6,6- d₈)isoindolin-2-yl)piperidine-2,6-dione |
| | 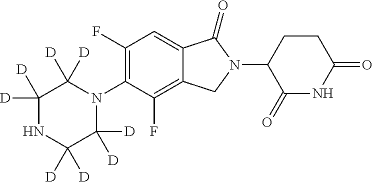 [SMILES: [2H]C1([2H])NC([2H])([2H])C([2H])([2H])N(c2c(F)cc3c(c2F)CN(C2CCC(=O)NC2=O)C3=O)C1([2H])[2H]] | 3-(4,6-difluoro-1-oxo-5- (piperazin-1-yl-2,2,3,3,5,5,6,6- d₈)isoindolin-2-yl)piperidine-2,6-dione |
| | 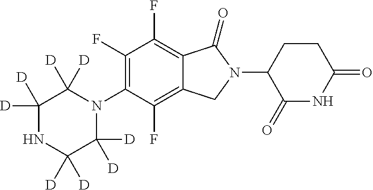 [SMILES: [2H]C1([2H])NC([2H])([2H])C([2H])([2H])N(c2c(F)c(F)c3c(c2F)CN(C2CCC(=O)NC2=O)C3=O)C1([2H])[2H]] | 3-(4,6,7-trifluoro-1-oxo-5- (piperazin-1-yl-2,2,3,3,5,5,6,6- d₈)isoindolin-2-yl)piperidine-2,6-dione |
| | 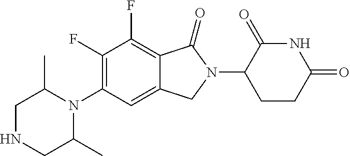 [SMILES: CC1CNCC(C)N1c1cc2c(c(F)c1F)C(=O)N(C1CCC(=O)NC1=O)C2] | 3-(5-(2,6-dimethylpiperazin-1-yl)- 6,7-difluoro-1- oxoisoindolin-2-yl)piperidine-2,6-dione |
| | 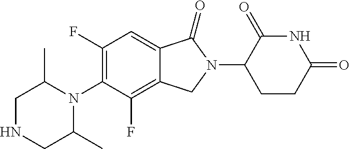 [SMILES: CC1CNCC(C)N1c1c(F)cc2c(c1F)CN(C1CCC(=O)NC1=O)C2=O] | 3-(5-(2,6-dimethylpiperazin-1-yl)- 4,6-difluoro-1- oxoisoindolin-2-yl)piperidine-2,6-dione |
| | 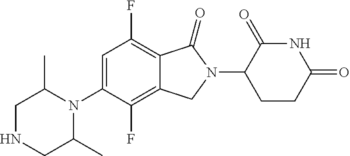 [SMILES: CC1CNCC(C)N1c1cc(F)c2c(c1F)CN(C1CCC(=O)NC1=O)C2=O] | 3-(5-(2,6-dimethylpiperazin-1-yl)- 4,7-difluoro-1- oxoisoindolin-2-yl)piperidine-2,6-dione |
| | 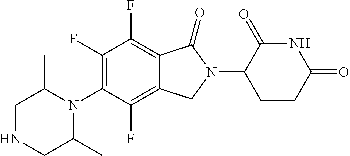 [SMILES: CC1CNCC(C)N1c1c(F)c(F)c2c(c1F)CN(C1CCC(=O)NC1=O)C2=O] | 3-(5-(2,6-dimethylpiperazin-1-yl)- 4,6,7-trifluoro-1- oxoisoindolin-2-yl)piperidine-2,6-dione |
| | 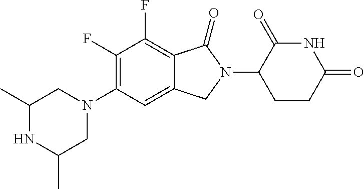 [SMILES: CC1CN(c2cc3c(c(F)c2F)C(=O)N(C2CCC(=O)NC2=O)C3)CC(C)N1] | 3-(5-(3,5-dimethylpiperazin-1-yl)- 6,7-difluoro-1- oxoisoindolin-2-yl)piperidine-2,6-dione |
| | 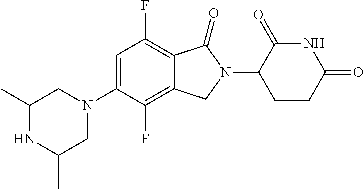 [SMILES: CC1CN(c2cc(F)c3c(c2F)CN(C2CCC(=O)NC2=O)C3=O)CC(C)N1] | 3-(5-(3,5-dimethylpiperazin-1-yl)- 4,7-difluoro-1- oxoisoindolin-2-yl)piperidine-2,6-dione |
| | 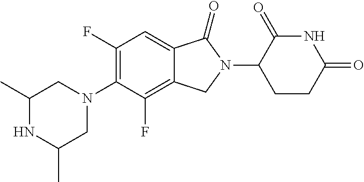 [SMILES: CC1CN(c2c(F)cc3c(c2F)CN(C2CCC(=O)NC2=O)C3=O)CC(C)N1] | 3-(5-(3,5-dimethylpiperazin-1-yl)- 4,6-difluoro-1- oxoisoindolin-2-yl)piperidine-2,6-dione |
| | 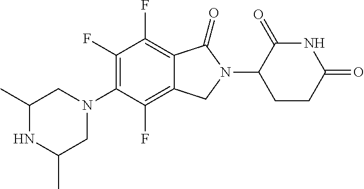 [SMILES: CC1CN(c2c(F)c(F)c3c(c2F)CN(C2CCC(=O)NC2=O)C3=O)CC(C)N1] | 3-(5-(3,5-dimethylpiperazin-1-yl)- 4,6,7-trifluoro-1- oxoisoindolin-2-yl)piperidine-2,6-dione |
| | 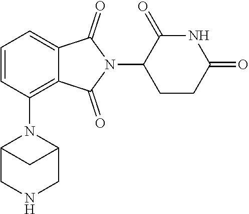 [SMILES: O=C1CCC(N2C(=O)c3cccc(N4C5CNCC4C5)c3C2=O)C(=O)N1] | 4-(3,6-diazabicyclo[3.1.1]heptan-6-yl)- 2-(2,6-dioxopiperidin- 3-yl)isoindoline-1,3-dione |
| | 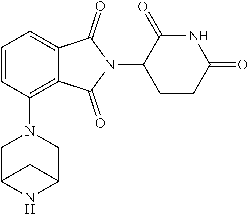 [SMILES: O=C1CCC(N2C(=O)c3cccc(N4CC5CC(C4)N5)c3C2=O)C(=O)N1] | 4-(3,6-diazabicyclo[3.1.1]heptan-3-yl)- 2-(2,6-dioxopiperidin- 3-yl)isoindoline-1,3-dione |
| | 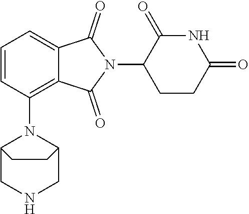 [SMILES: O=C1CCC(N2C(=O)c3cccc(N4C5CCC4CNC5)c3C2=O)C(=O)N1] | 4-((1R,5S)-3,8-diazabicyclo[3.2.1]octan-8-yl)- 2-(2,6- dioxopiperidin-3-yl)isoindoline-1,3-dione |
| | 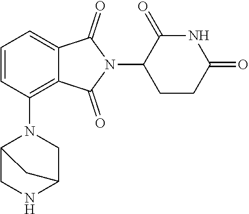 [SMILES: O=C1CCC(N2C(=O)c3cccc(N4CC5CC4CN5)c3C2=O)C(=O)N1] | 4-(2,5-diazabicyclo[2.2.1]heptan-2-yl)- 2-(2,6-dioxopiperidin- 3-yl)isoindoline-1,3-dione |
| | 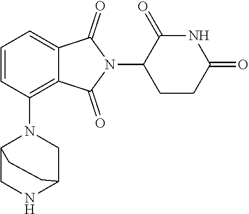 [SMILES: O=C1CCC(N2C(=O)c3cccc(N4CC5CCC4CN5)c3C2=O)C(=O)N1] | 4-(2,5-diazabicyclo[2.2.2]octan-2-yl)- 2-(2,6-dioxopiperidin-3- yl)isoindoline-1,3-dione |
| | 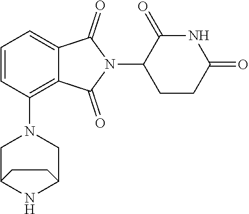 [SMILES: O=C1CCC(N2C(=O)c3cccc(N4CC5CCC(C4)N5)c3C2=O)C(=O)N1] | 4-((1R,5S)-3,8-diazabicyclo[3.2.1]octan-3-yl)- 2-(2,6- dioxopiperidin-3-yl)isoindoline-1,3-dione |
| | 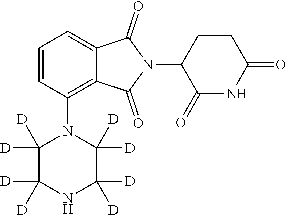 [SMILES: [2H]C1([2H])NC([2H])([2H])C([2H])([2H])N(c2cccc3c2C(=O)N(C2CCC(=O)NC2=O)C3=O)C1([2H])[2H]] | 2-(2,6-dioxopiperidin-3-yl)-4- (piperazin-1-yl-2,2,3,3,5,5,6,6- d₈)isoindoline-1,3-dione |
| | 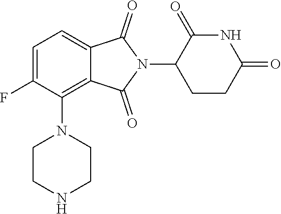 [SMILES: O=C1CCC(N2C(=O)c3ccc(F)c(N4CCNCC4)c3C2=O)C(=O)N1] | 2-(2,6-dioxopiperidin-3-yl)- 5-fluoro-4-(piperazin-1- yl)isoindoline-1,3-dione |
| | 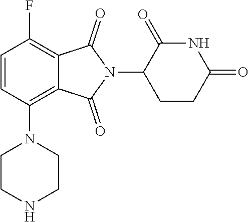 [SMILES: O=C1CCC(N2C(=O)c3c(F)ccc(N4CCNCC4)c3C2=O)C(=O)N1] | 2-(2,6-dioxopiperidin-3-yl)- 4-fluoro-7-(piperazin-1- yl)isoindoline-1,3-dione |
| | 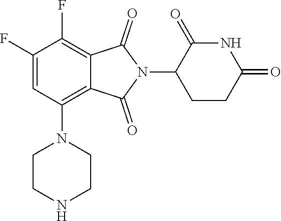 [SMILES: O=C1CCC(N2C(=O)c3c(N4CCNCC4)cc(F)c(F)c3C2=O)C(=O)N1] | 2-(2,6-dioxopiperidin-3-yl)- 4,5-difluoro-7-(piperazin-1- yl)isoindoline-1,3-dione |
| | 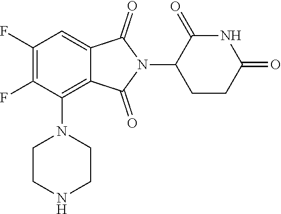 [SMILES: O=C1CCC(N2C(=O)c3cc(F)c(F)c(N4CCNCC4)c3C2=O)C(=O)N1] | 2-(2,6-dioxopiperidin-3-yl)- 5,6-difluoro-4-(piperazin-1- yl)isoindoline-1,3-dione |
| | 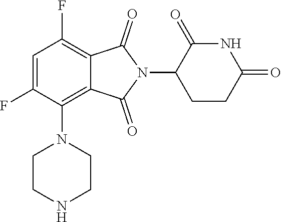 [SMILES: O=C1CCC(N2C(=O)c3c(F)cc(F)c(N4CCNCC4)c3C2=O)C(=O)N1] | 2-(2,6-dioxopiperidin-3-yl)- 5,7-difluoro-4-(piperazin-1- yl)isoindoline-1,3-dione |
| | 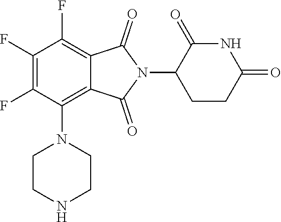 [SMILES: O=C1CCC(N2C(=O)c3c(F)c(F)c(F)c(N4CCNCC4)c3C2=O)C(=O)N1] | 2-(2,6-dioxopiperidin-3-yl)- 4,5,6-trifluoro-7-(piperazin-1- yl)isoindoline-1,3-dione |
| | 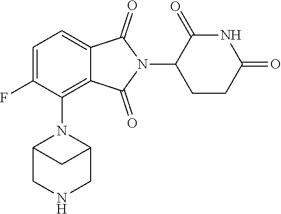 [SMILES: O=C1CCC(N2C(=O)c3ccc(F)c(N4C5CNCC4C5)c3C2=O)C(=O)N1] | 4-(3,6-diazabicyclo[3.1.1]heptan-6-yl)- 2-(2,6-dioxopiperidin- 3-yl)-5-fluoroisoindoline-1,3-dione |
| | 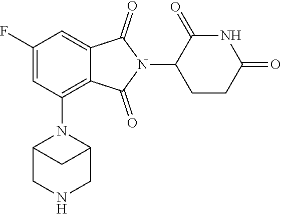 [SMILES: O=C1CCC(N2C(=O)c3cc(F)cc(N4C5CNCC4C5)c3C2=O)C(=O)N1] | 4-(3,6-diazabicyclo[3.1.1]heptan-6-yl)- 2-(2,6-dioxopiperidin- 3-yl)-6-fluoroisoindoline-1,3-dione |
| | 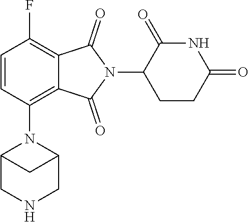 [SMILES: O=C1CCC(N2C(=O)c3c(F)ccc(N4C5CNCC4C5)c3C2=O)C(=O)N1] | 4-(3,6-diazabicyclo[3.1.1]heptan-6-yl)- 2-(2,6-dioxopiperidin- 3-yl)-7-fluoroisoindoline-1,3-dione |
| | 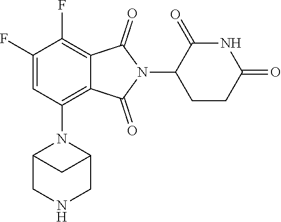 [SMILES: O=C1CCC(N2C(=O)c3c(N4C5CNCC4C5)cc(F)c(F)c3C2=O)C(=O)N1] | 7-(3,6-diazabicyclo[3.1.1]heptan-6-yl)- 2-(2,6-dioxopiperidin- 3-yl)-4,5-difluoroisoindoline-1,3-dione |
| | 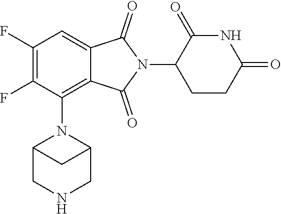 [SMILES: O=C1CCC(N2C(=O)c3cc(F)c(F)c(N4C5CNCC4C5)c3C2=O)C(=O)N1] | 4-(3,6-diazabicyclo[3.1.1]heptan-6-yl)- 2-(2,6-dioxopiperidin- 3-yl)-5,6-difluoroisoindoline-1,3-dione |
| | 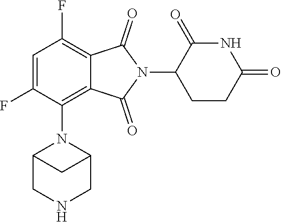 [SMILES: O=C1CCC(N2C(=O)c3c(F)cc(F)c(N4C5CNCC4C5)c3C2=O)C(=O)N1] | 4-(3,6-diazabicyclo[3.1.1]heptan-6-yl)- 2-(2,6-dioxopiperidin- 3-yl)-5,7-difluoroisoindoline-1,3-dione |
| | 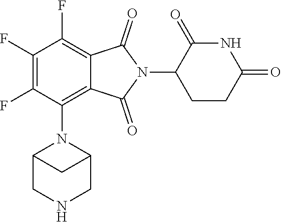 [SMILES: O=C1CCC(N2C(=O)c3c(F)c(F)c(F)c(N4C5CNCC4C5)c3C2=O)C(=O)N1] | 4-(3,6-diazabicyclo[3.1.1]heptan-6-yl)- 2-(2,6-dioxopiperidin- 3-yl)-5,6,7-trifluoroisoindoline-1,3-dione |
| | 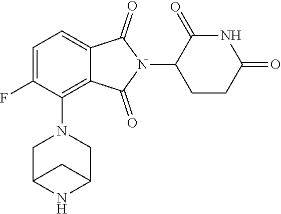 [SMILES: O=C1CCC(N2C(=O)c3ccc(F)c(N4CC5CC(C4)N5)c3C2=O)C(=O)N1] | 4-(3,6-diazabicyclo[3.1.1]heptan-3-yl)- 2-(2,6-dioxopiperidin- 3-yl)-5-fluoroisoindoline-1,3-dione |
| | 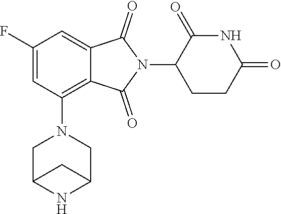 [SMILES: O=C1CCC(N2C(=O)c3cc(F)cc(N4CC5CC(C4)N5)c3C2=O)C(=O)N1] | 4-(3,6-diazabicyclo[3.1.1]heptan-3-yl)- 2-(2,6-dioxopiperidin- 3-yl)-6-fluoroisoindoline-1,3-dione |
| | 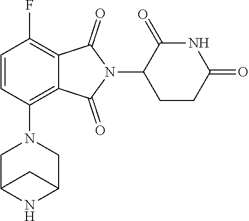 [SMILES: O=C1CCC(N2C(=O)c3c(F)ccc(N4CC5CC(C4)N5)c3C2=O)C(=O)N1] | 4-(3,6-diazabicyclo[3.1.1]heptan-3-yl)- 2-(2,6-dioxopiperidin- 3-yl)-7-fluoroisoindoline-1,3-dione |
| | 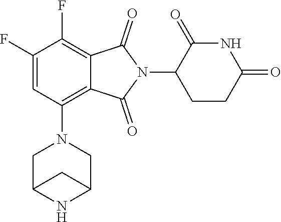 [SMILES: O=C1CCC(N2C(=O)c3c(N4CC5CC(C4)N5)cc(F)c(F)c3C2=O)C(=O)N1] | 7-(3,6-diazabicyclo[3.1.1]heptan-3-yl)- 2-(2,6-dioxopiperidin- 3-yl)-4,5-difluoroisoindoline-1,3-dione |
| | 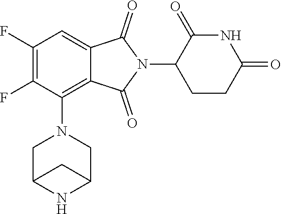 [SMILES: O=C1CCC(N2C(=O)c3cc(F)c(F)c(N4CC5CC(C4)N5)c3C2=O)C(=O)N1] | 4-(3,6-diazabicyclo[3.1.1]heptan-3-yl)- 2-(2,6-dioxopiperidin- 3-yl)-5,6-difluoroisoindoline-1,3-dione |
| | 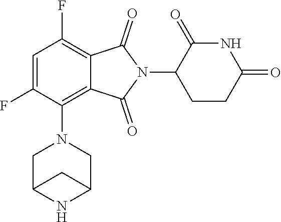 [SMILES: O=C1CCC(N2C(=O)c3c(F)cc(F)c(N4CC5CC(C4)N5)c3C2=O)C(=O)N1] | 4-(3,6-diazabicyclo[3.1.1]heptan-3-yl)- 2-(2,6-dioxopiperidin- 3-yl)-5,7-difluoroisoindoline-1,3-dione |
| | 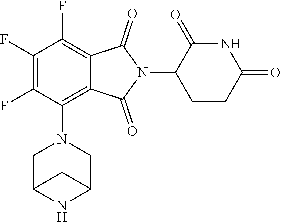 [SMILES: O=C1CCC(N2C(=O)c3c(F)c(F)c(F)c(N4CC5CC(C4)N5)c3C2=O)C(=O)N1] | 4-(3,6-diazabicyclo[3.1.1]heptan-3-yl)- 2-(2,6-dioxopiperidin- 3-yl)-5,6,7-trifluoroisoindoline-1,3-dione |
| | 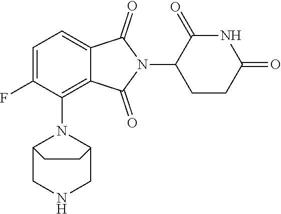 [SMILES: O=C1CCC(N2C(=O)c3ccc(F)c(N4C5CCC4CNC5)c3C2=O)C(=O)N1] | 4-((1R,5S)-3,8-diazabicyclo[3.2.1]octan-8-yl)- 2-(2,6-dioxopiperidin-3-yl)- 5-fluoroisoindoline-1,3-dione |
| | 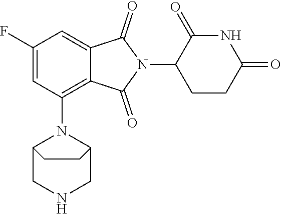 [SMILES: O=C1CCC(N2C(=O)c3cc(F)cc(N4C5CCC4CNC5)c3C2=O)C(=O)N1] | 4-((1R,5S)-3,8-diazabicyclo[3.2.1]octan-8-yl)- 2-(2,6- dioxopiperidin-3-yl)-6-fluoroisoindoline-1,3-dione |
| | 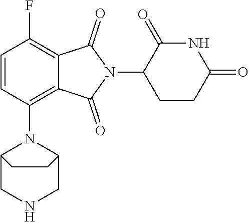 [SMILES: O=C1CCC(N2C(=O)c3c(F)ccc(N4C5CCC4CNC5)c3C2=O)C(=O)N1] | 4-((1R,5S)-3,8-diazabicyclo[3.2.1]octan-8-yl)- 2-(2,6- dioxopiperidin-3-yl)-7-fluoroisoindoline-1,3-dione |
| | 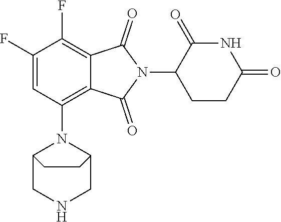 [SMILES: O=C1CCC(N2C(=O)c3c(N4C5CCC4CNC5)cc(F)c(F)c3C2=O)C(=O)N1] | 7-((1R,5S)-3,8-diazabicyclo[3.2.1]octan-8-yl)- 2-(2,6- dioxopiperidin-3-yl)-4,5-difluoroisoindoline-1,3-dione |
| | 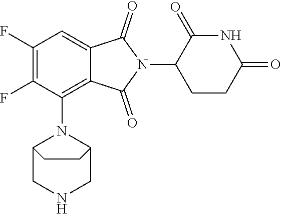 [SMILES: O=C1CCC(N2C(=O)c3cc(F)c(F)c(N4C5CCC4CNC5)c3C2=O)C(=O)N1] | 4-((1R,5S)-3,8-diazabicyclo[3.2.1]octan-8-yl)- 2-(2,6- dioxopiperidin-3-yl)-5,6-difluoroisoindoline-1,3-dione |
| | 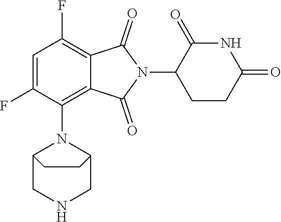 [SMILES: O=C1CCC(N2C(=O)c3c(F)cc(F)c(N4C5CCC4CNC5)c3C2=O)C(=O)N1] | 4-((1R,5S)-3,8-diazabicyclo[3.2.1]octan-8-yl)- 2-(2,6- dioxopiperidin-3-yl)-5,7-difluoroisoindoline-1,3-dione |
| | 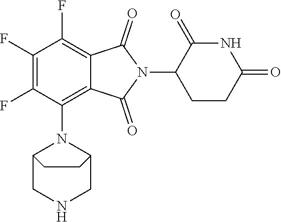 [SMILES: O=C1CCC(N2C(=O)c3c(F)c(F)c(F)c(N4C5CCC4CNC5)c3C2=O)C(=O)N1] | 4-((1R,5S)-3,8-diazabicyclo[3.2.1]octan-8-yl)- 2-(2,6- dioxopiperidin-3-yl)-5,6,7-trifluoroisoindoline-1,3-dione |
| | 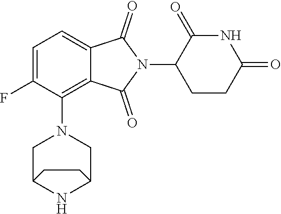 [SMILES: O=C1CCC(N2C(=O)c3ccc(F)c(N4CC5CCC(C4)N5)c3C2=O)C(=O)N1] | 4-((1R,5S)-3,8-diazabicyclo[3.2.1]octan-3-yl)- 2-(2,6- dioxopiperidin-3-yl)-5-fluoroisoindoline-1,3-dione |
| | 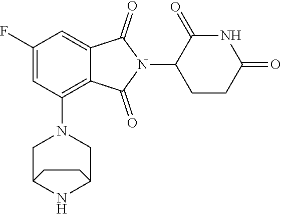 [SMILES: O=C1CCC(N2C(=O)c3cc(F)cc(N4CC5CCC(C4)N5)c3C2=O)C(=O)N1] | 4-((1R,5S)-3,8-diazabicyclo[3.2.1]octan-3-yl)- 2-(2,6- dioxopiperidin-3-yl)-6-fluoroisoindoline-1,3-dione |
| | 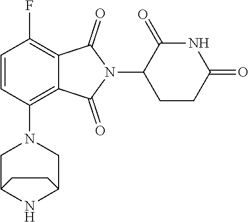 [SMILES: O=C1CCC(N2C(=O)c3c(F)ccc(N4CC5CCC(C4)N5)c3C2=O)C(=O)N1] | 4-((1R,5S)-3,8-diazabicyclo[3.2.1]octan-3-yl)- 2-(2,6- dioxopiperidin-3-yl)-7-fluoroisoindoline-1,3-dione |
| | 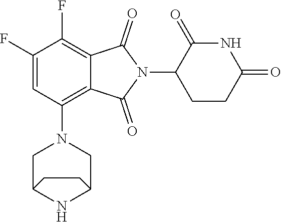 [SMILES: O=C1CCC(N2C(=O)c3c(N4CC5CCC(C4)N5)cc(F)c(F)c3C2=O)C(=O)N1] | 7-((1R,5S)-3,8-diazabicyclo[3.2.1]octan-3-yl)- 2-(2,6- dioxopiperidin-3-yl)-4,5-difluoroisoindoline-1,3-dione |
| | 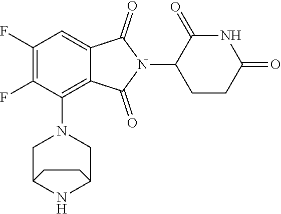 [SMILES: O=C1CCC(N2C(=O)c3cc(F)c(F)c(N4CC5CCC(C4)N5)c3C2=O)C(=O)N1] | 4-((1R,5S)-3,8-diazabicyclo[3.2.1]octan-3-yl)- 2-(2,6- dioxopiperidin-3-yl)-5,6-difluoroisoindoline-1,3-dione |
| | 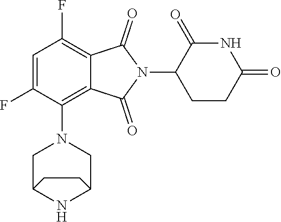 [SMILES: O=C1CCC(N2C(=O)c3c(F)cc(F)c(N4CC5CCC(C4)N5)c3C2=O)C(=O)N1] | 4-((1R,5S)-3,8-diazabicyclo[3.2.1]octan-3-yl)- 2-(2,6- dioxopiperidin-3-yl)-5,7-difluoroisoindoline-1,3-dione |
| | 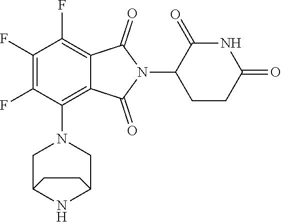 [SMILES: O=C1CCC(N2C(=O)c3c(F)c(F)c(F)c(N4CC5CCC(C4)N5)c3C2=O)C(=O)N1] | 4-((1R,5S)-3,8-diazabicyclo[3.2.1]octan-3-yl)- 2-(2,6-dioxopiperidin-3-yl)- 5,6,7-trifluoroisoindoline-1,3-dione |
| | 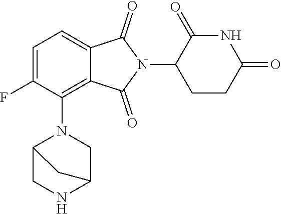 [SMILES: O=C1CCC(N2C(=O)c3ccc(F)c(N4CC5CC4CN5)c3C2=O)C(=O)N1] | 4-(2,5-diazabicyclo[2.2.1]heptan-2-yl)- 2-(2,6-dioxopiperidin- 3-yl)-5-fluoroisoindoline-1,3-dione |
| | 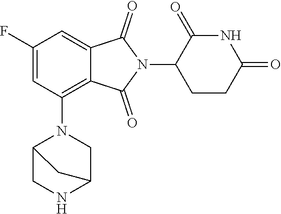 [SMILES: O=C1CCC(N2C(=O)c3cc(F)cc(N4CC5CC4CN5)c3C2=O)C(=O)N1] | 4-(2,5-diazabicyclo[2.2.1]heptan-2-yl)- 2-(2,6-dioxopiperidin- 3-yl)-6-fluoroisoindoline-1,3-dione |
| | 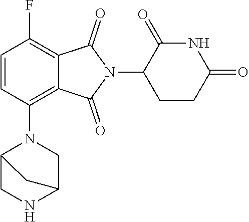 [SMILES: O=C1CCC(N2C(=O)c3c(F)ccc(N4CC5CC4CN5)c3C2=O)C(=O)N1] | 4-(2,5-diazabicyclo[2.2.1]heptan-2-yl)- 2-(2,6-dioxopiperidin- 3-yl)-7-fluoroisoindoline-1,3-dione |
| | 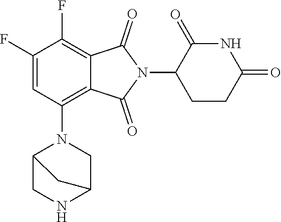 [SMILES: O=C1CCC(N2C(=O)c3c(N4CC5CC4CN5)cc(F)c(F)c3C2=O)C(=O)N1] | 7-(2,5-diazabicyclo[2.2.1]heptan-2-yl)- 2-(2,6-dioxopiperidin- 3-yl)-4,5-difluoroisoindoline-1,3-dione |
| | 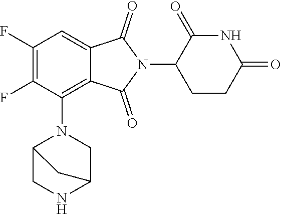 [SMILES: O=C1CCC(N2C(=O)c3cc(F)c(F)c(N4CC5CC4CN5)c3C2=O)C(=O)N1] | 4-(2,5-diazabicyclo[2.2.1]heptan-2-yl)- 2-(2,6-dioxopiperidin- 3-yl)-5,6-difluoroisoindoline-1,3-dione |
| | 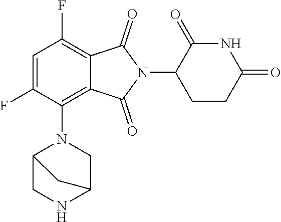 [SMILES: O=C1CCC(N2C(=O)c3c(F)cc(F)c(N4CC5CC4CN5)c3C2=O)C(=O)N1] | 4-(2,5-diazabicyclo[2.2.1]heptan-2-yl)- 2-(2,6-dioxopiperidin- 3-yl)-5,7-difluoroisoindoline-1,3-dione |
| | 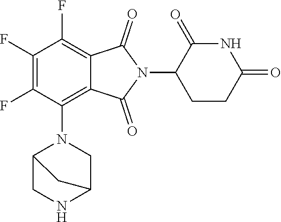 [SMILES: O=C1CCC(N2C(=O)c3c(F)c(F)c(F)c(N4CC5CC4CN5)c3C2=O)C(=O)N1] | 4-(2,5-diazabicyclo[2.2.1]heptan-2-yl)- 2-(2,6-dioxopiperidin- 3-yl)-5,6,7-trifluoroisoindoline-1,3-dione |
| | 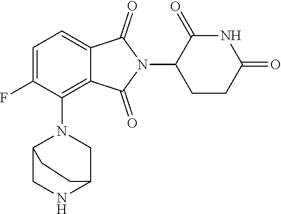 [SMILES: O=C1CCC(N2C(=O)c3ccc(F)c(N4CC5CCC4CN5)c3C2=O)C(=O)N1] | 4-(2,5-diazabicyclo[2.2.2]octan-2-yl)- 2-(2,6-dioxopiperidin-3- yl)-5-fluoroisoindoline-1,3-dione |
| | 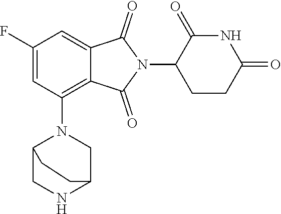 [SMILES: O=C1CCC(N2C(=O)c3cc(F)cc(N4CC5CCC4CN5)c3C2=O)C(=O)N1] | 4-(2,5-diazabicyclo[2.2.2]octan-2-yl)- 2-(2,6-dioxopiperidin-3- yl)-6-fluoroisoindoline-1,3-dione |
| | 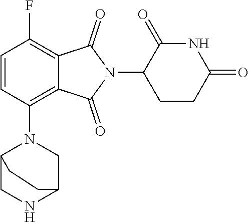 [SMILES: O=C1CCC(N2C(=O)c3c(F)ccc(N4CC5CCC4CN5)c3C2=O)C(=O)N1] | 4-(2,5-diazabicyclo[2.2.2]octan-2-yl)- 2-(2,6-dioxopiperidin-3- yl)-7-fluoroisoindoline-1,3-dione |
| | 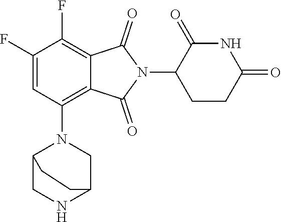 [SMILES: O=C1CCC(N2C(=O)c3c(N4CC5CCC4CN5)cc(F)c(F)c3C2=O)C(=O)N1] | 7-(2,5-diazabicyclo[2.2.2]octan-2-yl)- 2-(2,6-dioxopiperidin-3- yl)-4,5-difluoroisoindoline-1,3-dione |
| | 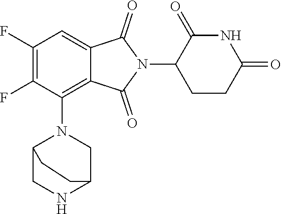 [SMILES: O=C1CCC(N2C(=O)c3cc(F)c(F)c(N4CC5CCC4CN5)c3C2=O)C(=O)N1] | 4-(2,5-diazabicyclo[2.2.2]octan-2-yl)- 2-(2,6-dioxopiperidin-3- yl)-5,6-difluoroisoindoline-1,3-dione |
| | 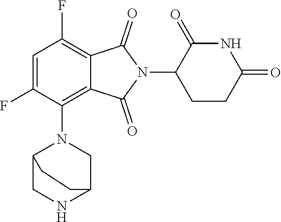 [SMILES: O=C1CCC(N2C(=O)c3c(F)cc(F)c(N4CC5CCC4CN5)c3C2=O)C(=O)N1] | 4-(2,5-diazabicyclo[2.2.2]octan-2-yl)- 2-(2,6-dioxopiperidin-3- yl)-5,7-difluoroisoindoline-1,3-dione |
| | 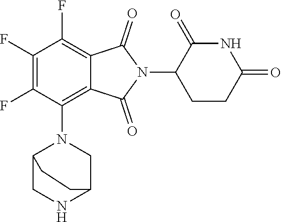 [SMILES: O=C1CCC(N2C(=O)c3c(F)c(F)c(F)c(N4CC5CCC4CN5)c3C2=O)C(=O)N1] | 4-(2,5-diazabicyclo[2.2.2]octan-2-yl)- 2-(2,6-dioxopiperidin-3- yl)-5,6,7-trifluoroisoindoline-1,3-dione |
| | 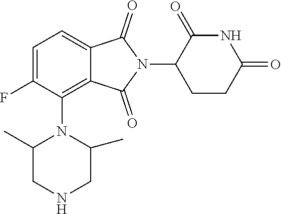 [SMILES: CC1CNCC(C)N1c1c(F)ccc2c1C(=O)N(C1CCC(=O)NC1=O)C2=O] | 4-(2,6-dimethylpiperazin-1-yl)- 2-(2,6-dioxopiperidin-3-yl)-5- fluoroisoindoline-1,3-dione |
| | 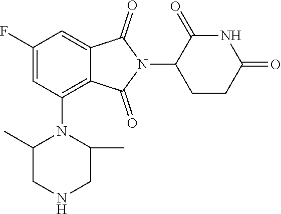 [SMILES: CC1CNCC(C)N1c1cc(F)cc2c1C(=O)N(C1CCC(=O)NC1=O)C2=O] | 4-(2,6-dimethylpiperazin-1-yl)- 2-(2,6-dioxopiperidin-3-yl)-6- fluoroisoindoline-1,3-dione |
| | 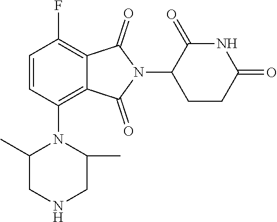 [SMILES: CC1CNCC(C)N1c1ccc(F)c2c1C(=O)N(C1CCC(=O)NC1=O)C2=O] | 4-(2,6-dimethylpiperazin-1-yl)- 2-(2,6-dioxopiperidin-3-yl)-7- fluoroisoindoline-1,3-dione |
| | 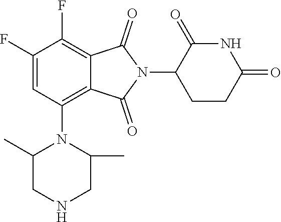 [SMILES: CC1CNCC(C)N1c1cc(F)c(F)c2c1C(=O)N(C1CCC(=O)NC1=O)C2=O] | 7-(2,6-dimethylpiperazin-1-yl)- 2-(2,6-dioxopiperidin-3-yl)- 4,5-difluoroisoindoline-1,3-dione |
| | 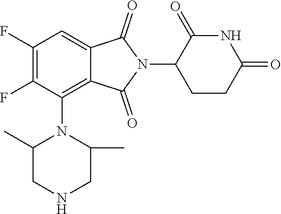 [SMILES: CC1CNCC(C)N1c1c(F)c(F)cc2c1C(=O)N(C1CCC(=O)NC1=O)C2=O] | 4-(2,6-dimethylpiperazin-1-yl)- 2-(2,6-dioxopiperidin-3-yl)- 5,6-difluoroisoindoline-1,3-dione |
| | 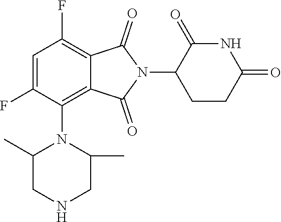 [SMILES: CC1CNCC(C)N1c1c(F)cc(F)c2c1C(=O)N(C1CCC(=O)NC1=O)C2=O] | 4-(2,6-dimethylpiperazin-1-yl)- 2-(2,6-dioxopiperidin-3-yl)- 5,7-difluoroisoindoline-1,3-dione |
| | 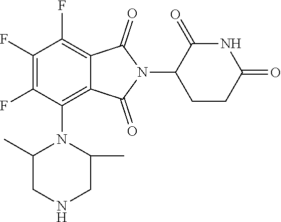 [SMILES: CC1CNCC(C)N1c1c(F)c(F)c(F)c2c1C(=O)N(C1CCC(=O)NC1=O)C2=O] | 4-(2,6-dimethylpiperazin-1-yl)- 2-(2,6-dioxopiperidin-3-yl)- 5,6,7-trifluoroisoindoline-1,3-dione |
| | 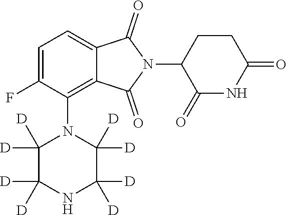 [SMILES: [2H]C1([2H])NC([2H])([2H])C([2H])([2H])N(c2c(F)ccc3c2C(=O)N(C2CCC(=O)NC2=O)C3=O)C1([2H])[2H]] | 2-(2,6-dioxopiperidin-3-yl)- 5-fluoro-4-(piperazin-1-yl- 2,2,3,3,5,5,6,6-d₈)isoindoline-1,3-dione |
| | 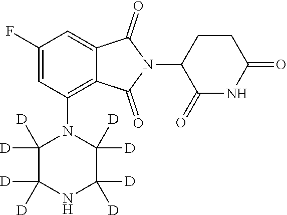 [SMILES: [2H]C1([2H])NC([2H])([2H])C([2H])([2H])N(c2cc(F)cc3c2C(=O)N(C2CCC(=O)NC2=O)C3=O)C1([2H])[2H]] | 2-(2,6-dioxopiperidin-3-yl)- 6-fluoro-4-(piperazin-1-yl- 2,2,3,3,5,5,6,6-d₈)isoindoline-1,3-dione |
| | 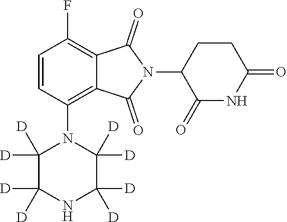 [SMILES: [2H]C1([2H])NC([2H])([2H])C([2H])([2H])N(c2ccc(F)c3c2C(=O)N(C2CCC(=O)NC2=O)C3=O)C1([2H])[2H]] | 2-(2,6-dioxopiperidin-3-yl)- 4-fluoro-7-(piperazin-1-yl- 2,2,3,3,5,5,6,6-d₈)isoindoline-1,3-dione |
| | 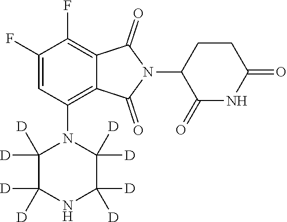 [SMILES: [2H]C1([2H])NC([2H])([2H])C([2H])([2H])N(c2cc(F)c(F)c3c2C(=O)N(C2CCC(=O)NC2=O)C3=O)C1([2H])[2H]] | 2-(2,6-dioxopiperidin-3-yl)- 4,5-difluoro-7-(piperazin-1-yl- 2,2,3,3,5,5,6,6-d₈)isoindoline-1,3-dione |
| | 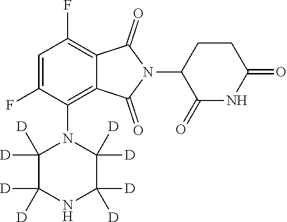 [SMILES: [2H]C1([2H])NC([2H])([2H])C([2H])([2H])N(c2c(F)cc(F)c3c2C(=O)N(C2CCC(=O)NC2=O)C3=O)C1([2H])[2H]] | 2-(2,6-dioxopiperidin-3-yl)- 5,7-difluoro-4-(piperazin-1-yl- 2,2,3,3,5,5,6,6-d₈)isoindoline-1,3-dione |
| | 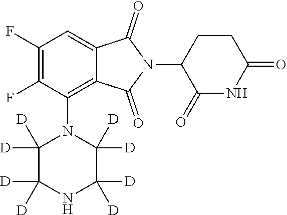 [SMILES: [2H]C1([2H])NC([2H])([2H])C([2H])([2H])N(c2c(F)c(F)cc3c2C(=O)N(C2CCC(=O)NC2=O)C3=O)C1([2H])[2H]] | 2-(2,6-dioxopiperidin-3-yl)- 5,6-difluoro-4-(piperazin-1-yl- 2,2,3,3,5,5,6,6-d₈)isoindoline-1,3-dione |
| | 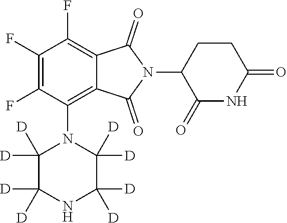 [SMILES: [2H]C1([2H])NC([2H])([2H])C([2H])([2H])N(c2c(F)c(F)c(F)c3c2C(=O)N(C2CCC(=O)NC2=O)C3=O)C1([2H])[2H]] | 2-(2,6-dioxopiperidin-3-yl)- 4,5,6-trifluoro-7-(piperazin-1-yl- 2,2,3,3,5,5,6,6-d₈)isoindoline-1,3-dione |
| | 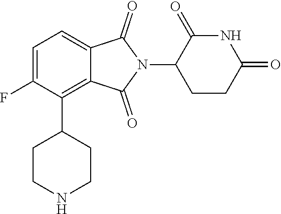 [SMILES: O=C1CCC(N2C(=O)c3ccc(F)c(C4CCNCC4)c3C2=O)C(=O)N1] | 2-(2,6-dioxopiperidin-3-yl)- 5-fluoro-4-(piperidin-4- yl)isoindoline-1,3-dione |
| | 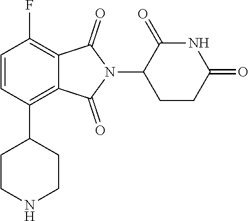 [SMILES: O=C1CCC(N2C(=O)c3c(F)ccc(C4CCNCC4)c3C2=O)C(=O)N1] | 2-(2,6-dioxopiperidin-3-yl)- 4-fluoro-7-(piperidin-4- yl)isoindoline-1,3-dione |
| | 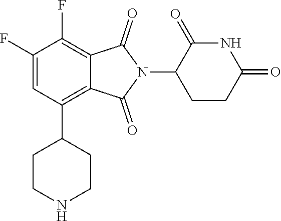 [SMILES: O=C1CCC(N2C(=O)c3c(C4CCNCC4)cc(F)c(F)c3C2=O)C(=O)N1] | 2-(2,6-dioxopiperidin-3-yl)- 4,5-difluoro-7-(piperidin-4- yl)isoindoline-1,3-dione |
| | 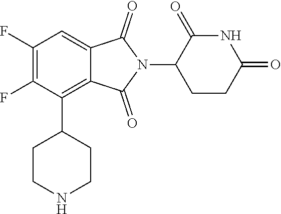 [SMILES: O=C1CCC(N2C(=O)c3cc(F)c(F)c(C4CCNCC4)c3C2=O)C(=O)N1] | 2-(2,6-dioxopiperidin-3-yl)- 5,6-difluoro-4-(piperidin-4- yl)isoindoline-1,3-dione |
| | 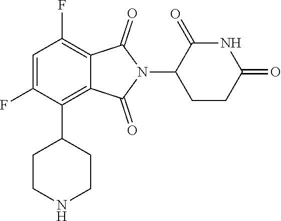 [SMILES: O=C1CCC(N2C(=O)c3c(F)cc(F)c(C4CCNCC4)c3C2=O)C(=O)N1] | 2-(2,6-dioxopiperidin-3-yl)- 5,7-difluoro-4-(piperidin-4- yl)isoindoline-1,3-dione |
| | 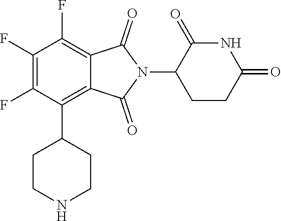 [SMILES: O=C1CCC(N2C(=O)c3c(F)c(F)c(F)c(C4CCNCC4)c3C2=O)C(=O)N1] | 2-(2,6-dioxopiperidin-3-yl)- 4,5,6-trifluoro-7-(piperidin-4- yl)isoindoline-1,3-dione |
| | 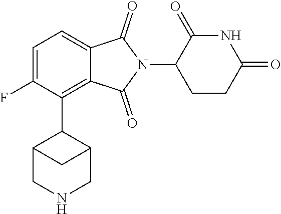 [SMILES: O=C1CCC(N2C(=O)c3ccc(F)c(C4C5CNCC4C5)c3C2=O)C(=O)N1] | 4-(3-azabicyclo[3.1.1]heptan-6-yl)- 2-(2,6-dioxopiperidin-3- yl)-5-fluoroisoindoline-1,3-dione |
| | 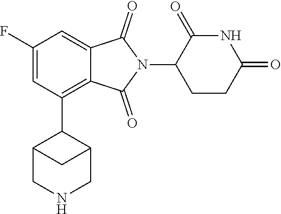 [SMILES: O=C1CCC(N2C(=O)c3cc(F)cc(C4C5CNCC4C5)c3C2=O)C(=O)N1] | 4-(3-azabicyclo[3.1.1]heptan-6-yl)- 2-(2,6-dioxopiperidin-3- yl)-6-fluoroisoindoline-1,3-dione |
| | 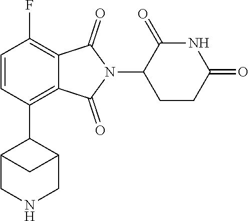 [SMILES: O=C1CCC(N2C(=O)c3c(F)ccc(C4C5CNCC4C5)c3C2=O)C(=O)N1] | 4-(3-azabicyclo[3.1.1]heptan-6-yl)- 2-(2,6-dioxopiperidin-3- yl)-7-fluoroisoindoline-1,3-dione |
| | 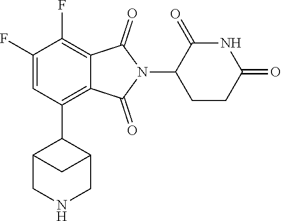 [SMILES: O=C1CCC(N2C(=O)c3c(C4C5CNCC4C5)cc(F)c(F)c3C2=O)C(=O)N1] | 7-(3-azabicyclo[3.1.1]heptan-6-yl)- 2-(2,6-dioxopiperidin-3- yl)-4,5-difluoroisoindoline-1,3-dione |
| | 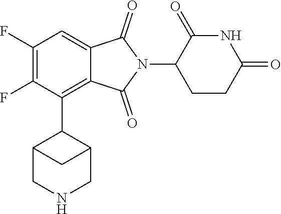 [SMILES: O=C1CCC(N2C(=O)c3cc(F)c(F)c(C4C5CNCC4C5)c3C2=O)C(=O)N1] | 4-(3-azabicyclo[3.1.1]heptan-6-yl)- 2-(2,6-dioxopiperidin-3- yl)-5,6-difluoroisoindoline-1,3-dione |
| | 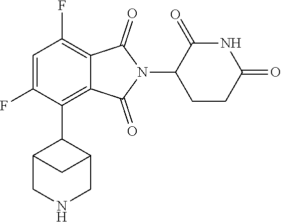 [SMILES: O=C1CCC(N2C(=O)c3c(F)cc(F)c(C4C5CNCC4C5)c3C2=O)C(=O)N1] | 4-(3-azabicyclo[3.1.1]heptan-6-yl)- 2-(2,6-dioxopiperidin-3- yl)-5,7-difluoroisoindoline-1,3-dione |
| | 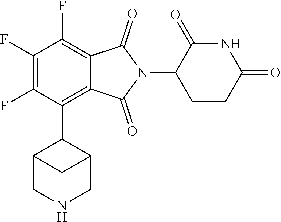 [SMILES: O=C1CCC(N2C(=O)c3c(F)c(F)c(F)c(C4C5CNCC4C5)c3C2=O)C(=O)N1] | 4-(3-azabicyclo[3.1.1]heptan-6-yl)- 2-(2,6-dioxopiperidin-3- yl)-5,6,7-trifluoroisoindoline-1,3-dione |
| | 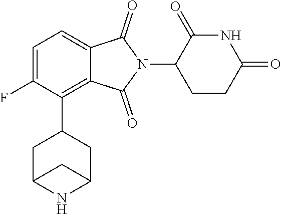 [SMILES: O=C1CCC(N2C(=O)c3ccc(F)c(C4CC5CC(C4)N5)c3C2=O)C(=O)N1] | 4-(6-azabicyclo[3.1.1]heptan-3-yl)- 2-(2,6-dioxopiperidin-3- yl)-5-fluoroisoindoline-1,3-dione |
| | 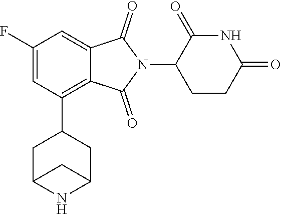 [SMILES: O=C1CCC(N2C(=O)c3cc(F)cc(C4CC5CC(C4)N5)c3C2=O)C(=O)N1] | 4-(6-azabicyclo[3.1.1]heptan-3-yl)- 2-(2,6-dioxopiperidin-3- yl)-6-fluoroisoindoline-1,3-dione |
| | 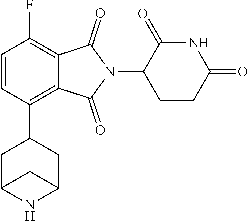 [SMILES: O=C1CCC(N2C(=O)c3c(F)ccc(C4CC5CC(C4)N5)c3C2=O)C(=O)N1] | 4-(6-azabicyclo[3.1.1]heptan-3-yl)- 2-(2,6-dioxopiperidin-3- yl)-7-fluoroisoindoline-1,3-dione |
| | 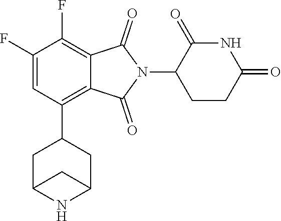 [SMILES: O=C1CCC(N2C(=O)c3c(C4CC5CC(C4)N5)cc(F)c(F)c3C2=O)C(=O)N1] | 7-(6-azabicyclo[3.1.1]heptan-3-yl)- 2-(2,6-dioxopiperidin-3- yl)-4,5-difluoroisoindoline-1,3-dione |
| | 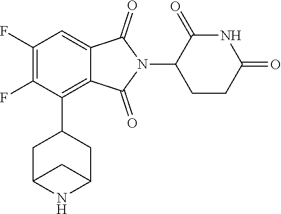 [SMILES: O=C1CCC(N2C(=O)c3cc(F)c(F)c(C4CC5CC(C4)N5)c3C2=O)C(=O)N1] | 4-(6-azabicyclo[3.1.1]heptan-3-yl)- 2-(2,6-dioxopiperidin-3- yl)-5,6-difluoroisoindoline-1,3-dione |
| | 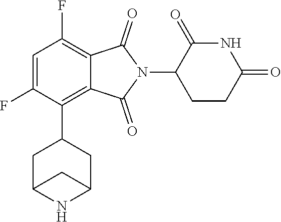 [SMILES: O=C1CCC(N2C(=O)c3c(F)cc(F)c(C4CC5CC(C4)N5)c3C2=O)C(=O)N1] | 4-(6-azabicyclo[3.1.1]heptan-3-yl)- 2-(2,6-dioxopiperidin-3- yl)-5,7-difluoroisoindoline-1,3-dione |
| | 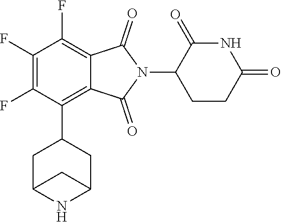 [SMILES: O=C1CCC(N2C(=O)c3c(F)c(F)c(F)c(C4CC5CC(C4)N5)c3C2=O)C(=O)N1] | 4-(6-azabicyclo[3.1.1]heptan-3-yl)- 2-(2,6-dioxopiperidin-3- yl)-5,6,7-trifluoroisoindoline-1,3-dione |
| | 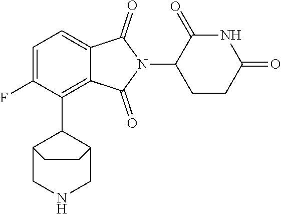 [SMILES: O=C1CCC(N2C(=O)c3ccc(F)c(C4C5CCC4CNC5)c3C2=O)C(=O)N1] | 4-((1R,5S,8r)-3-azabicyclo[3.2.1]octan-8-yl)- 2-(2,6- dioxopiperidin-3-yl)-5-fluoroisoindoline-1,3-dione |
| | 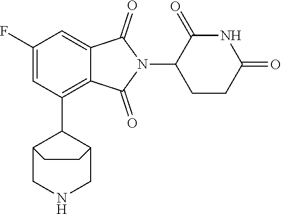 [SMILES: O=C1CCC(N2C(=O)c3cc(F)cc(C4C5CCC4CNC5)c3C2=O)C(=O)N1] | 4-((1R,5S,8r)-3-azabicyclo[3.2.1]octan-8-yl)- 2-(2,6- dioxopiperidin-3-yl)-6-fluoroisoindoline-1,3-dione |
| | 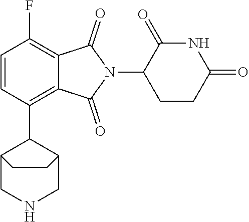 [SMILES: O=C1CCC(N2C(=O)c3c(F)ccc(C4C5CCC4CNC5)c3C2=O)C(=O)N1] | 4-((1R,5S,8r)-3-azabicyclo[3.2.1]octan-8-yl)- 2-(2,6- dioxopiperidin-3-yl)-7-fluoroisoindoline-1,3-dione |
| | 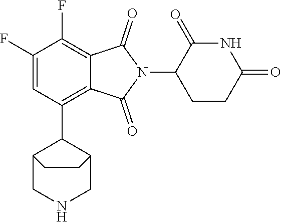 [SMILES: O=C1CCC(N2C(=O)c3c(C4C5CCC4CNC5)cc(F)c(F)c3C2=O)C(=O)N1] | 7-((1R,5S,8r)-3-azabicyclo[3.2.1]octan-8-yl)- 2-(2,6-dioxopiperidin-3-yl)- 4,5-difluoroisoindoline-1,3-dione |
| | 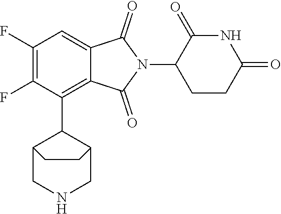 [SMILES: O=C1CCC(N2C(=O)c3cc(F)c(F)c(C4C5CCC4CNC5)c3C2=O)C(=O)N1] | 4-((1R,5S,8r)-3-azabicyclo[3.2.1]octan-8-yl)- 2-(2,6-dioxopiperidin-3-yl)- 5,6-difluoroisoindoline-1,3-dione |
| | 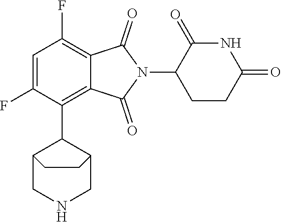 [SMILES: O=C1CCC(N2C(=O)c3c(F)cc(F)c(C4C5CCC4CNC5)c3C2=O)C(=O)N1] | 4-((1R,5S,8r)-3-azabicyclo[3.2.1]octan-8-yl)- 2-(2,6- dioxopiperidin-3-yl)-5,7- difluoroisoindoline-1,3-dione |
| | 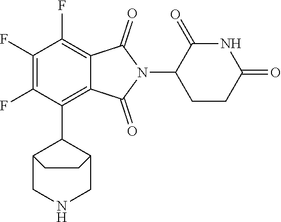 [SMILES: O=C1CCC(N2C(=O)c3c(F)c(F)c(F)c(C4C5CCC4CNC5)c3C2=O)C(=O)N1] | 4-((1R,5S,8r)-3-azabicyclo[3.2.1]octan-8-yl)- 2-(2,6- dioxopiperidin-3-yl)-5,6,7- trifluoroisoindoline-1,3-dione |
| | 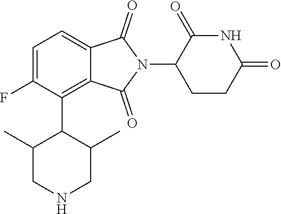 [SMILES: CC1CNCC(C)C1c1c(F)ccc2c1C(=O)N(C1CCC(=O)NC1=O)C2=O] | 4-(3,5-dimethylpiperidin-4-yl)- 2-(2,6-dioxopiperidin-3-yl)-5- fluoroisoindoline-1,3-dione |
| | 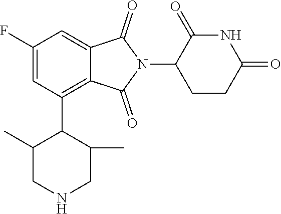 [SMILES: CC1CNCC(C)C1c1cc(F)cc2c1C(=O)N(C1CCC(=O)NC1=O)C2=O] | 4-(3,5-dimethylpiperidin-4-yl)- 2-(2,6-dioxopiperidin-3-yl)-6- fluoroisoindoline-1,3-dione |
| | 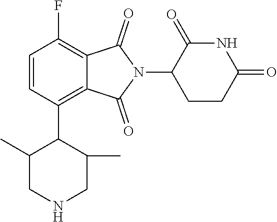 [SMILES: CC1CNCC(C)C1c1ccc(F)c2c1C(=O)N(C1CCC(=O)NC1=O)C2=O] | 4-(3,5-dimethylpiperidin-4-yl)- 2-(2,6-dioxopiperidin-3-yl)-7- fluoroisoindoline-1,3-dione |
| | 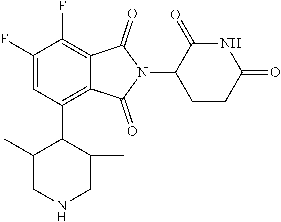 [SMILES: CC1CNCC(C)C1c1cc(F)c(F)c2c1C(=O)N(C1CCC(=O)NC1=O)C2=O] | 7-(3,5-dimethylpiperidin-4-yl)- 2-(2,6-dioxopiperidin-3-yl)- 4,5-difluoroisoindoline-1,3-dione |
| | 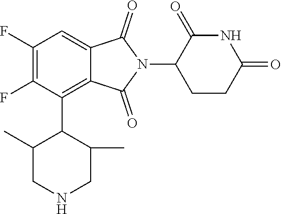 [SMILES: CC1CNCC(C)C1c1c(F)c(F)cc2c1C(=O)N(C1CCC(=O)NC1=O)C2=O] | 4-(3,5-dimethylpiperidin-4-yl)- 2-(2,6-dioxopiperidin-3-yl)- 5,6-difluoroisoindoline-1,3-dione |
| | 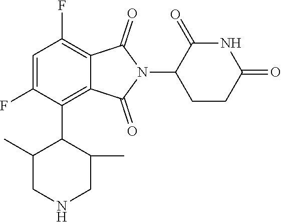 [SMILES: CC1CNCC(C)C1c1c(F)cc(F)c2c1C(=O)N(C1CCC(=O)NC1=O)C2=O] | 4-(3,5-dimethylpiperidin-4-yl)- 2-(2,6-dioxopiperidin-3-yl)- 5,7-difluoroisoindoline-1,3-dione |
| | 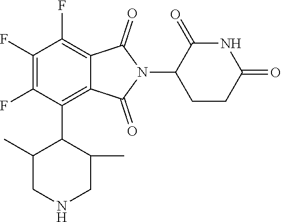 [SMILES: CC1CNCC(C)C1c1c(F)c(F)c(F)c2c1C(=O)N(C1CCC(=O)NC1=O)C2=O] | 4-(3,5-dimethylpiperidin-4-yl)- 2-(2,6-dioxopiperidin-3-yl)- 5,6,7-trifluoroisoindoline-1,3-dione |
| | 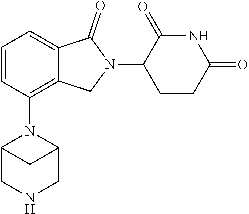 [SMILES: O=C1CCC(N2Cc3c(cccc3N3C4CNCC3C4)C2=O)C(=O)N1] | 3-(4-(3,6-diazabicyclo[3.1.1]heptan-6-yl)- 1-oxoisoindolin-2- yl)piperidine-2,6-dione |
| | 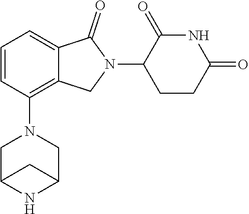 [SMILES: O=C1CCC(N2Cc3c(cccc3N3CC4CC(C3)N4)C2=O)C(=O)N1] | 3-(4-(3,6-diazabicyclo[3.1.1]heptan-3-yl)- 1-oxoisoindolin-2- yl)piperidine-2,6-dione |
| | 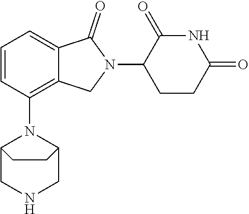 [SMILES: O=C1CCC(N2Cc3c(cccc3N3C4CCC3CNC4)C2=O)C(=O)N1] | 3-(4-((1R,5S)-3,8- diazabicyclo[3.2.1]octan-8-yl)-1- oxoisoindolin-2-yl)piperidine-2,6-dione |
| | 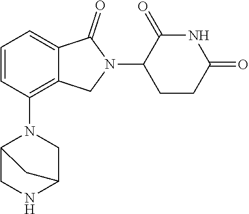 [SMILES: O=C1CCC(N2Cc3c(cccc3N3CC4CC3CN4)C2=O)C(=O)N1] | 3-(4-(2,5-diazabicyclo[2.2.1]heptan-2-yl)- 1-oxoisoindolin-2- yl)piperidine-2,6-dione |
| | 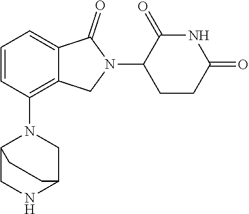 [SMILES: O=C1CCC(N2Cc3c(cccc3N3CC4CCC3CN4)C2=O)C(=O)N1] | 3-(4-(2,5-diazabicyclo[2.2.2]octan-2-yl)- 1-oxoisoindolin-2- yl)piperidine-2,6-dione |
| | 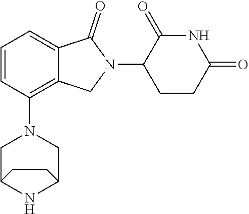 [SMILES: O=C1CCC(N2Cc3c(cccc3N3CC4CCC(C3)N4)C2=O)C(=O)N1] | 3-(4-((1R,5S)-3,8-diazabicyclo[3.2.1]octan-3-yl)- 1-oxoisoindolin-2-yl)piperidine-2,6-dione |
| | 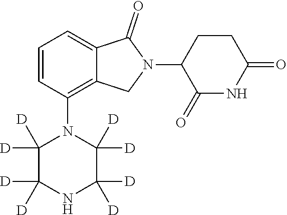 [SMILES: [2H]C1([2H])NC([2H])([2H])C([2H])([2H])N(c2cccc3c2CN(C2CCC(=O)NC2=O)C3=O)C1([2H])[2H]] | 3-(1-oxo-4- (piperazin-1-yl-2,2,3,3,5,5,6,6-d₈)isoindolin-2- yl)piperidine-2,6-dione |
| | 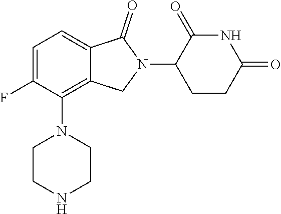 [SMILES: O=C1CCC(N2Cc3c(ccc(F)c3N3CCNCC3)C2=O)C(=O)N1] | 3-(5-fluoro-1-oxo-4- (piperazin-1-yl)isoindolin-2-yl)piperidine- 2,6-dione |
| | 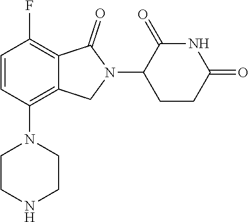 [SMILES: O=C1CCC(N2Cc3c(N4CCNCC4)ccc(F)c3C2=O)C(=O)N1] | 3-(7-fluoro-1-oxo-4- (piperazin-1-yl)isoindolin-2-yl)piperidine- 2,6-dione |
| | 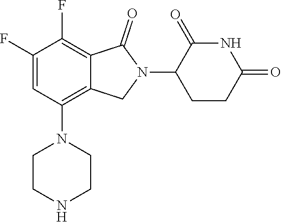 [SMILES: O=C1CCC(N2Cc3c(N4CCNCC4)cc(F)c(F)c3C2=O)C(=O)N1] | 3-(6,7-difluoro-1-oxo-4- (piperazin-1-yl)isoindolin-2- yl)piperidine-2,6-dione |
| | 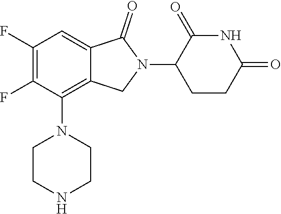 [SMILES: O=C1CCC(N2Cc3c(cc(F)c(F)c3N3CCNCC3)C2=O)C(=O)N1] | 3-(5,6-difluoro-1-oxo-4- (piperazin-1-yl)isoindolin-2- yl)piperidine-2,6-dione |
| | 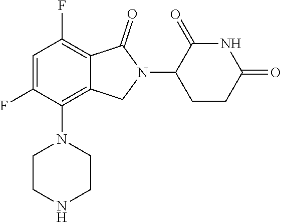 [SMILES: O=C1CCC(N2Cc3c(c(F)cc(F)c3N3CCNCC3)C2=O)C(=O)N1] | 3-(5,7-difluoro-1-oxo-4- (piperazin-1-yl)isoindolin-2- yl)piperidine-2,6-dione |
| | 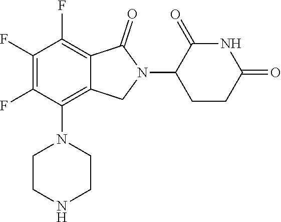 [SMILES: O=C1CCC(N2Cc3c(c(F)c(F)c(F)c3N3CCNCC3)C2=O)C(=O)N1] | 3-(5,6,7-trifluoro-1-oxo-4- (piperazin-1-yl)isoindolin-2- yl)piperidine-2,6-dione |
| | 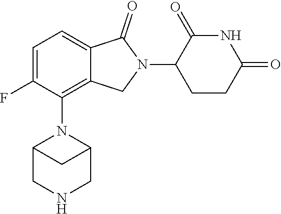 [SMILES: O=C1CCC(N2Cc3c(ccc(F)c3N3C4CNCC3C4)C2=O)C(=O)N1] | 3-(4-(3,6-diazabicyclo[3.1.1]heptan-6-yl)- 5-fluoro-1- oxoisoindolin-2-yl)piperidine-2,6-dione |
| | 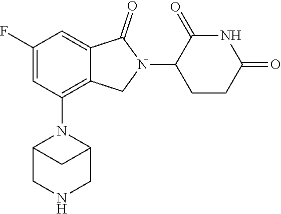 [SMILES: O=C1CCC(N2Cc3c(cc(F)cc3N3C4CNCC3C4)C2=O)C(=O)N1] | 3-(4-(3,6-diazabicyclo[3.1.1]heptan-6-yl)- 6-fluoro-1- oxoisoindolin-2-yl)piperidine-2,6-dione |
| | 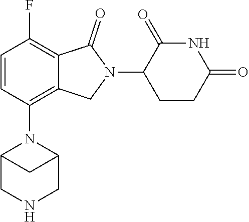 [SMILES: O=C1CCC(N2Cc3c(N4C5CNCC4C5)ccc(F)c3C2=O)C(=O)N1] | 3-(4-(3,6-diazabicyclo[3.1.1]heptan-6-yl)- 7-fluoro-1- oxoisoindolin-2-yl)piperidine-2,6-dione |
| | 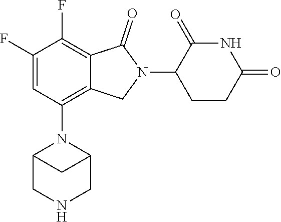 [SMILES: O=C1CCC(N2Cc3c(N4C5CNCC4C5)cc(F)c(F)c3C2=O)C(=O)N1] | 3-(4-(3,6-diazabicyclo[3.1.1]heptan-6-yl)- 6,7-difluoro-1- oxoisoindolin-2-yl)piperidine-2,6-dione |
| | 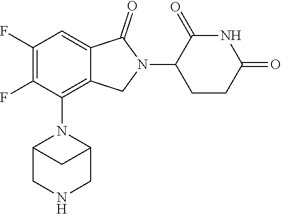 [SMILES: O=C1CCC(N2Cc3c(cc(F)c(F)c3N3C4CNCC3C4)C2=O)C(=O)N1] | 3-(4-(3,6-diazabicyclo[3.1.1]heptan-6-yl)- 5,6-difluoro-1- oxoisoindolin-2-yl)piperidine-2,6-dione |
| | 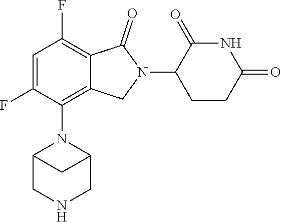 [SMILES: O=C1CCC(N2Cc3c(c(F)cc(F)c3N3C4CNCC3C4)C2=O)C(=O)N1] | 3-(4-(3,6-diazabicyclo[3.1.1]heptan-6-yl)- 5,7-difluoro-1- oxoisoindolin-2-yl)piperidine-2,6-dione |
| | 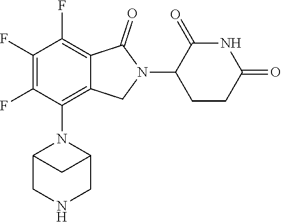 [SMILES: O=C1CCC(N2Cc3c(c(F)c(F)c(F)c3N3C4CNCC3C4)C2=O)C(=O)N1] | 3-(4-(3,6-diazabicyclo[3.1.1]heptan-6-yl)- 5,6,7-trifluoro-1- oxoisoindolin-2-yl)piperidine-2,6-dione |
| | 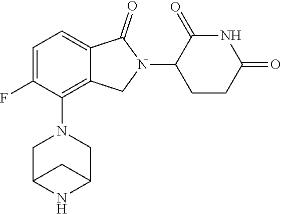 [SMILES: O=C1CCC(N2Cc3c(ccc(F)c3N3CC4CC(C3)N4)C2=O)C(=O)N1] | 3-(4-(3,6-diazabicyclo[3.1.1]heptan-3-yl)- 5-fluoro-1- oxoisoindolin-2-yl)piperidine-2,6-dione |
| | 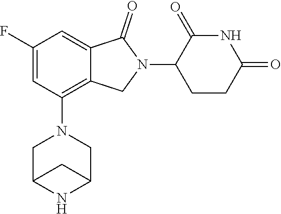 [SMILES: O=C1CCC(N2Cc3c(cc(F)cc3N3CC4CC(C3)N4)C2=O)C(=O)N1] | 3-(4-(3,6-diazabicyclo[3.1.1]heptan-3-yl)- 6-fluoro-1- oxoisoindolin-2-yl)piperidine-2,6-dione |
| | 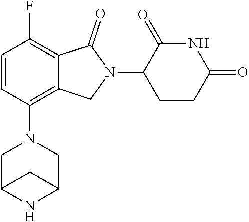 [SMILES: O=C1CCC(N2Cc3c(N4CC5CC(C4)N5)ccc(F)c3C2=O)C(=O)N1] | 3-(4-(3,6-diazabicyclo[3.1.1]heptan-3-yl)- 7-fluoro-1- oxoisoindolin-2-yl)piperidine-2,6-dione |
| | 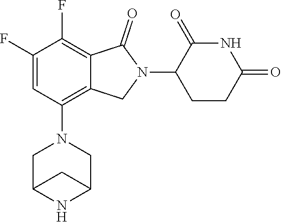 [SMILES: O=C1CCC(N2Cc3c(N4CC5CC(C4)N5)cc(F)c(F)c3C2=O)C(=O)N1] | 3-(4-(3,6-diazabicyclo[3.1.1]heptan-3-yl)- 6,7-difluoro-1- oxoisoindolin-2-yl)piperidine-2,6-dione |
| | 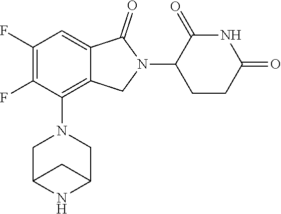 [SMILES: O=C1CCC(N2Cc3c(cc(F)c(F)c3N3CC4CC(C3)N4)C2=O)C(=O)N1] | 3-(4-(3,6-diazabicyclo[3.1.1]heptan-3-yl)- 5,6-difluoro-1- oxoisoindolin-2-yl)piperidine-2,6-dione |
| | 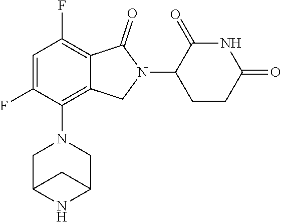 [SMILES: O=C1CCC(N2Cc3c(c(F)cc(F)c3N3CC4CC(C3)N4)C2=O)C(=O)N1] | 3-(4-(3,6-diazabicyclo[3.1.1]heptan-3-yl)- 5,7-difluoro-1- oxoisoindolin-2-yl)piperidine-2,6-dione |
| | 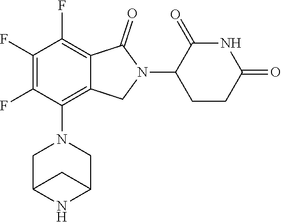 [SMILES: O=C1CCC(N2Cc3c(c(F)c(F)c(F)c3N3CC4CC(C3)N4)C2=O)C(=O)N1] | 3-(4-(3,6-diazabicyclo[3.1.1]heptan-3-yl)- 5,6,7-trifluoro-1- oxoisoindolin-2-yl)piperidine-2,6-dione |
| | 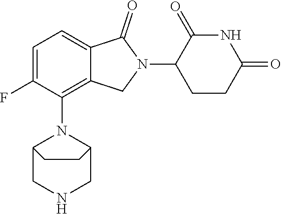 [SMILES: O=C1CCC(N2Cc3c(ccc(F)c3N3C4CCC3CNC4)C2=O)C(=O)N1] | 3-(4-((1R,5S)-3,8-diazabicyclo[3.2.1]octan-8-yl)- 5-fluoro-1- oxoisoindolin-2-yl)piperidine-2,6-dione |
| | 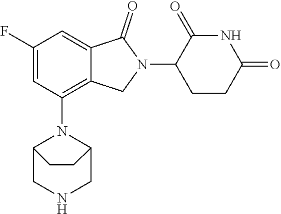 [SMILES: O=C1CCC(N2Cc3c(cc(F)cc3N3C4CCC3CNC4)C2=O)C(=O)N1] | 3-(4-((1R,5S)-3,8-diazabicyclo[3.2.1]octan-8-yl)- 6-fluoro-1- oxoisoindolin-2-yl)piperidine-2,6-dione |
| | 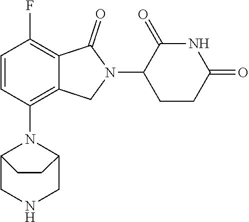 [SMILES: O=C1CCC(N2Cc3c(N4C5CCC4CNC5)ccc(F)c3C2=O)C(=O)N1] | 3-(4-((1R,5S)-3,8-diazabicyclo[3.2.1]octan-8-yl)- 7-fluoro-1- oxoisoindolin-2-yl)piperidine-2,6-dione |
| | 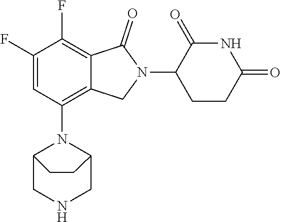 [SMILES: O=C1CCC(N2Cc3c(N4C5CCC4CNC5)cc(F)c(F)c3C2=O)C(=O)N1] | 3-(4-((1R,5S)-3,8-diazabicyclo[3.2.1]octan-8-yl)- 6,7-difluoro- 1-oxoisoindolin-2-yl)piperidine-2,6-dione |
| | 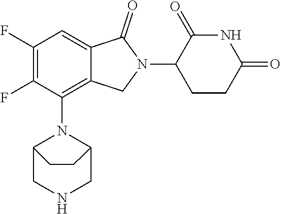 [SMILES: O=C1CCC(N2Cc3c(cc(F)c(F)c3N3C4CCC3CNC4)C2=O)C(=O)N1] | 3-(4-((1R,5S)-3,8-diazabicyclo[3.2.1]octan-8-yl)- 5,6-difluoro- 1-oxoisoindolin-2-yl)piperidine-2,6-dione |
| | 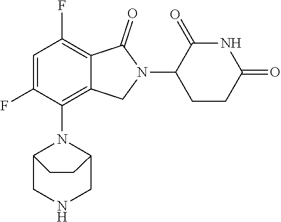 [SMILES: O=C1CCC(N2Cc3c(c(F)cc(F)c3N3C4CCC3CNC4)C2=O)C(=O)N1] | 3-(4-((1R,5S)-3,8-diazabicyclo[3.2.1]octan-8-yl)- 5,7-difluoro- 1-oxoisoindolin-2-yl)piperidine-2,6-dione |
| | 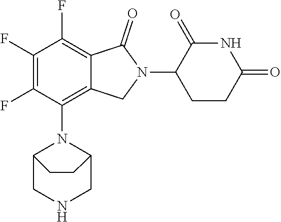 [SMILES: O=C1CCC(N2Cc3c(c(F)c(F)c(F)c3N3C4CCC3CNC4)C2=O)C(=O)N1] | 3-(4-((1R,5S)-3,8-diazabicyclo[3.2.1]octan-8-yl)- 5,6,7- trifluoro-1-oxoisoindolin-2-yl)piperidine-2,6-dione |
| | 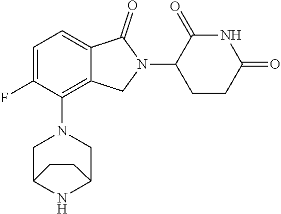 [SMILES: O=C1CCC(N2Cc3c(ccc(F)c3N3CC4CCC(C3)N4)C2=O)C(=O)N1] | 3-(4-((1R,5S)-3,8-diazabicyclo[3.2.1]octan-3-yl)- 5-fluoro-1- oxoisoindolin-2-yl)piperidine-2,6-dione |
| | 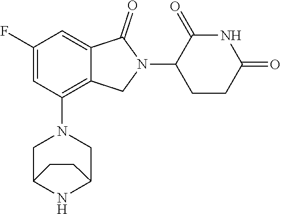 [SMILES: O=C1CCC(N2Cc3c(cc(F)cc3N3CC4CCC(C3)N4)C2=O)C(=O)N1] | 3-(4-((1R,5S)-3,8-diazabicyclo[3.2.1]octan-3-yl)- 6-fluoro-1- oxoisoindolin-2-yl)piperidine-2,6-dione |
| | 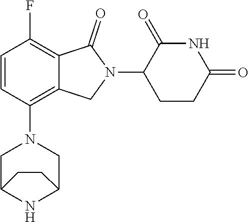 [SMILES: O=C1CCC(N2Cc3c(N4CC5CCC(C4)N5)ccc(F)c3C2=O)C(=O)N1] | 3-(4-((1R,5S)-3,8-diazabicyclo[3.2.1]octan-3-yl)- 7-fluoro-1- oxoisoindolin-2-yl)piperidine-2,6-dione |
| | 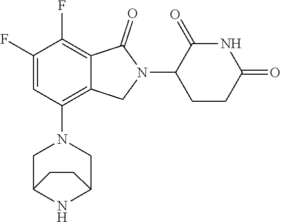 [SMILES: O=C1CCC(N2Cc3c(N4CC5CCC(C4)N5)cc(F)c(F)c3C2=O)C(=O)N1] | 3-(4-((1R,5S)-3,8-diazabicyclo[3.2.1]octan-3-yl)- 6,7-difluoro- 1-oxoisoindolin-2-yl)piperidine-2,6-dione |
| | 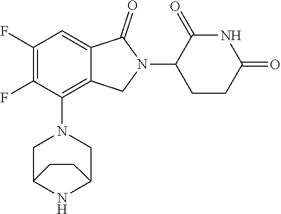 [SMILES: O=C1CCC(N2Cc3c(cc(F)c(F)c3N3CC4CCC(C3)N4)C2=O)C(=O)N1] | 3-(4-((1R,5S)-3,8-diazabicyclo[3.2.1]octan-3-yl)- 5,6-difluoro- 1-oxoisoindolin-2-yl)piperidine-2,6-dione |
| | 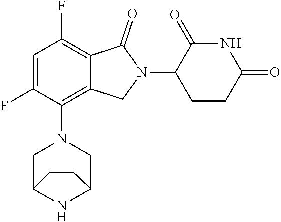 [SMILES: O=C1CCC(N2Cc3c(c(F)cc(F)c3N3CC4CCC(C3)N4)C2=O)C(=O)N1] | 3-(4-((1R,5S)-3,8-diazabicyclo[3.2.1]octan-3-yl)- 5,7-difluoro- 1-oxoisoindolin-2-yl)piperidine-2,6-dione |
| | 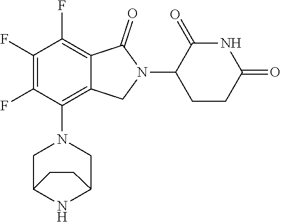 [SMILES: O=C1CCC(N2Cc3c(c(F)c(F)c(F)c3N3CC4CCC(C3)N4)C2=O)C(=O)N1] | 3-(4-((1R,5S)-3,8-diazabicyclo[3.2.1]octan-3-yl)- 5,6,7- trifluoro-1-oxoisoindolin-2-yl)piperidine-2,6-dione |
| | 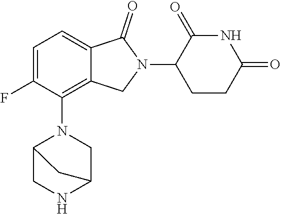 [SMILES: O=C1CCC(N2Cc3c(ccc(F)c3N3CC4CC3CN4)C2=O)C(=O)N1] | 3-(4-(2,5-diazabicyclo[2.2.1]heptan-2-yl)- 5-fluoro-1- oxoisoindolin-2-yl)piperidine-2,6-dione |
| | 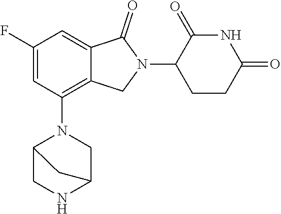 [SMILES: O=C1CCC(N2Cc3c(cc(F)cc3N3CC4CC3CN4)C2=O)C(=O)N1] | 3-(4-(2,5-diazabicyclo[2.2.1]heptan-2-yl)- 6-fluoro-1- oxoisoindolin-2-yl)piperidine-2,6-dione |
| | 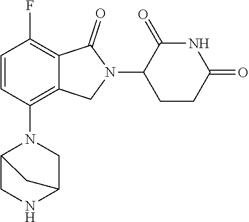 [SMILES: O=C1CCC(N2Cc3c(N4CC5CC4CN5)ccc(F)c3C2=O)C(=O)N1] | 3-(4-(2,5-diazabicyclo[2.2.1]heptan-2-yl)- 7-fluoro-1- oxoisoindolin-2-yl)piperidine-2,6-dione |
| | 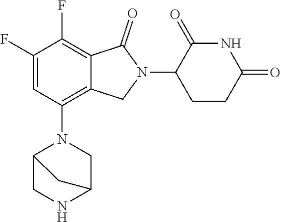 [SMILES: O=C1CCC(N2Cc3c(N4CC5CC4CN5)cc(F)c(F)c3C2=O)C(=O)N1] | 3-(4-(2,5-diazabicyclo[2.2.1]heptan-2-yl)- 6,7-difluoro-1- oxoisoindolin-2-yl)piperidine-2,6-dione |
| | 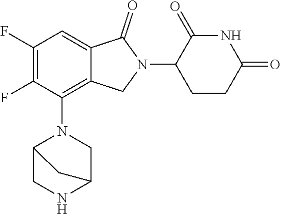 [SMILES: O=C1CCC(N2Cc3c(cc(F)c(F)c3N3CC4CC3CN4)C2=O)C(=O)N1] | 3-(4-(2,5-diazabicyclo[2.2.1]heptan-2-yl)- 5,6-difluoro-1- oxoisoindolin-2-yl)piperidine-2,6-dione |
| | 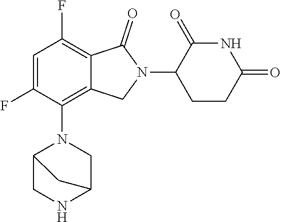 [SMILES: O=C1CCC(N2Cc3c(c(F)cc(F)c3N3CC4CC3CN4)C2=O)C(=O)N1] | 3-(4-(2,5-diazabicyclo[2.2.1]heptan-2-yl)- 5,7-difluoro-1- oxoisoindolin-2-yl)piperidine-2,6-dione |
| | 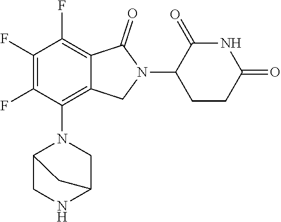 [SMILES: O=C1CCC(N2Cc3c(c(F)c(F)c(F)c3N3CC4CC3CN4)C2=O)C(=O)N1] | 3-(4-(2,5-diazabicyclo[2.2.1]heptan-2-yl)- 5,6,7-trifluoro-1- oxoisoindolin-2-yl)piperidine-2,6-dione |
| | 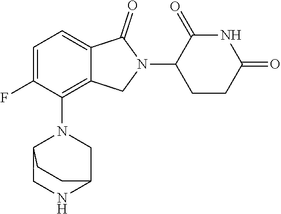 [SMILES: O=C1CCC(N2Cc3c(ccc(F)c3N3CC4CCC3CN4)C2=O)C(=O)N1] | 3-(4-(2,5-diazabicyclo[2.2.2]octan-2-yl)- 5-fluoro-1- oxoisoindolin-2-yl)piperidine-2,6-dione |
| | 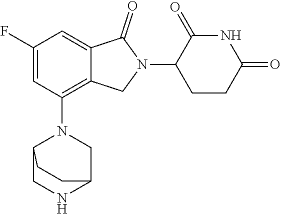 [SMILES: O=C1CCC(N2Cc3c(cc(F)cc3N3CC4CCC3CN4)C2=O)C(=O)N1] | 3-(4-(2,5-diazabicyclo[2.2.2]octan-2-yl)- 6-fluoro-1- oxoisoindolin-2-yl)piperidine-2,6-dione |
| | 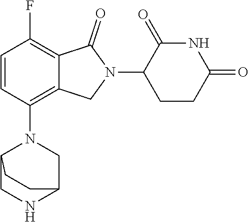 [SMILES: O=C1CCC(N2Cc3c(N4CC5CCC4CN5)ccc(F)c3C2=O)C(=O)N1] | 3-(4-(2,5-diazabicyclo[2.2.2]octan-2-yl)- 7-fluoro-1- oxoisoindolin-2-yl)piperidine-2,6-dione |
| | 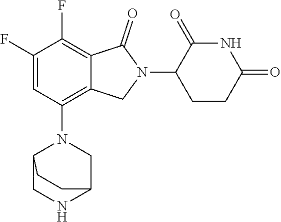 [SMILES: O=C1CCC(N2Cc3c(N4CC5CCC4CN5)cc(F)c(F)c3C2=O)C(=O)N1] | 3-(4-(2,5-diazabicyclo[2.2.2]octan-2-yl)- 6,7-difluoro-1- oxoisoindolin-2-yl)piperidine-2,6-dione |
| | 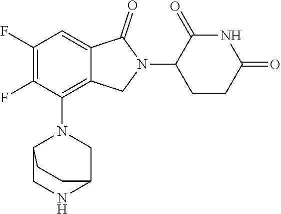 [SMILES: O=C1CCC(N2Cc3c(cc(F)c(F)c3N3CC4CCC3CN4)C2=O)C(=O)N1] | 3-(4-(2,5-diazabicyclo[2.2.2]octan-2-yl)- 5,6-difluoro-1- oxoisoindolin-2-yl)piperidine-2,6-dione |
| | 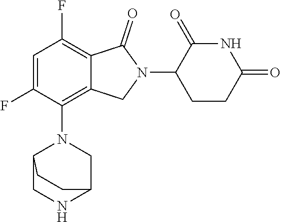 [SMILES: O=C1CCC(N2Cc3c(c(F)cc(F)c3N3CC4CCC3CN4)C2=O)C(=O)N1] | 3-(4-(2,5-diazabicyclo[2.2.2]octan-2-yl)- 5,7-difluoro-1- oxoisoindolin-2-yl)piperidine-2,6-dione |
| | 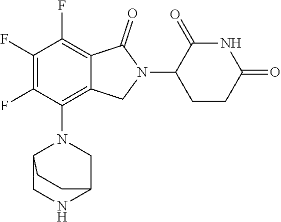 [SMILES: O=C1CCC(N2Cc3c(c(F)c(F)c(F)c3N3CC4CCC3CN4)C2=O)C(=O)N1] | 3-(4-(2,5-diazabicyclo[2.2.2]octan-2-yl)- 5,6,7-trifluoro-1- oxoisoindolin-2-yl)piperidine-2,6-dione |
| | 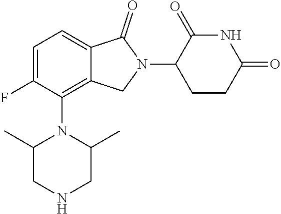 [SMILES: CC1CNCC(C)N1c1c(F)ccc2c1CN(C1CCC(=O)NC1=O)C2=O] | 3-(4-(2,6-dimethylpiperazin-1-yl)- 5-fluoro-1-oxoisoindolin-2- yl)piperidine-2,6-dione |
| | 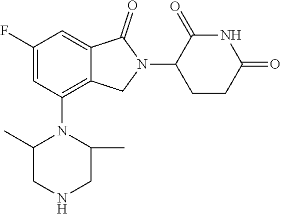 [SMILES: CC1CNCC(C)N1c1cc(F)cc2c1CN(C1CCC(=O)NC1=O)C2=O] | 3-(4-(2,6-dimethylpiperazin-1-yl)- 6-fluoro-1-oxoisoindolin-2- yl)piperidine-2,6-dione |
| | 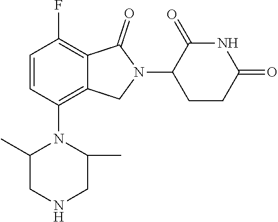 [SMILES: CC1CNCC(C)N1c1ccc(F)c2c1CN(C1CCC(=O)NC1=O)C2=O] | 3-(4-(2,6-dimethylpiperazin-1-yl)- 7-fluoro-1-oxoisoindolin-2- yl)piperidine-2,6-dione |
| | 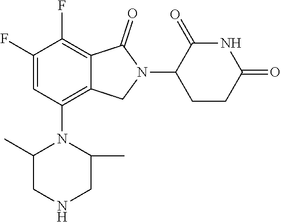 [SMILES: CC1CNCC(C)N1c1cc(F)c(F)c2c1CN(C1CCC(=O)NC1=O)C2=O] | 3-(4-(2,6-dimethylpiperazin-1-yl)- 6,7-difluoro-1- oxoisoindolin-2-yl)piperidine-2,6-dione |
| | 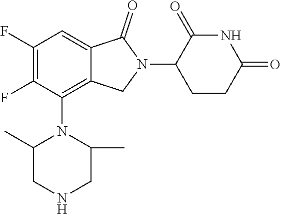 [SMILES: CC1CNCC(C)N1c1c(F)c(F)cc2c1CN(C1CCC(=O)NC1=O)C2=O] | 3-(4-(2,6-dimethylpiperazin-1-yl)- 5,6-difluoro-1- oxoisoindolin-2-yl)piperidine-2,6-dione |
| | 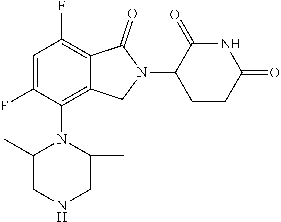 [SMILES: CC1CNCC(C)N1c1c(F)cc(F)c2c1CN(C1CCC(=O)NC1=O)C2=O] | 3-(4-(2,6-dimethylpiperazin-1-yl)- 5,7-difluoro-1- oxoisoindolin-2-yl)piperidine-2,6-dione |
| | 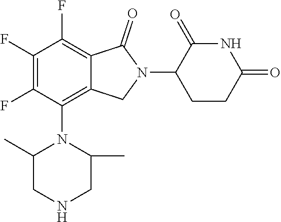 [SMILES: CC1CNCC(C)N1c1c(F)c(F)c(F)c2c1CN(C1CCC(=O)NC1=O)C2=O] | 3-(4-(2,6-dimethylpiperazin-1-yl)- 5,6,7-trifluoro-1- oxoisoindolin-2-yl)piperidine-2,6-dione |
| | 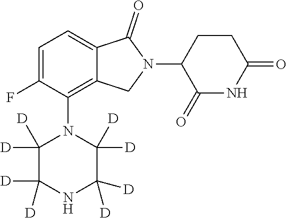 [SMILES: [2H]C1([2H])NC([2H])([2H])C([2H])([2H])N(c2c(F)ccc3c2CN(C2CCC(=O)NC2=O)C3=O)C1([2H])[2H]] | 3-(5-fluoro-1-oxo-4- (piperazin-1-yl-2,2,3,3,5,5,6,6- d₈)isoindolin-2-yl)piperidine-2,6-dione |
| | 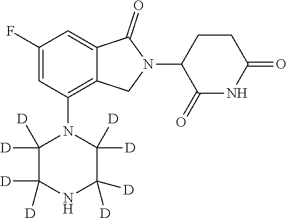 [SMILES: [2H]C1([2H])NC([2H])([2H])C([2H])([2H])N(c2cc(F)cc3c2CN(C2CCC(=O)NC2=O)C3=O)C1([2H])[2H]] | 3-(6-fluoro-1-oxo-4- (piperazin-1-yl-2,2,3,3,5,5,6,6- d₈)isoindolin-2-yl)piperidine-2,6-dione |
| | 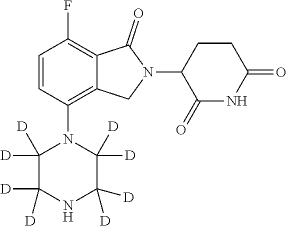 [SMILES: [2H]C1([2H])NC([2H])([2H])C([2H])([2H])N(c2ccc(F)c3c2CN(C2CCC(=O)NC2=O)C3=O)C1([2H])[2H]] | 3-(7-fluoro-1-oxo-4- (piperazin-1-yl-2,2,3,3,5,5,6,6- d₈)isoindolin-2-yl)piperidine-2,6-dione |
| | 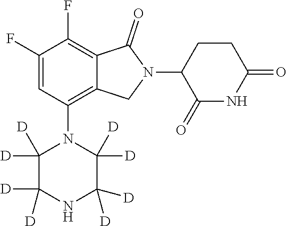 [SMILES: [2H]C1([2H])NC([2H])([2H])C([2H])([2H])N(c2cc(F)c(F)c3c2CN(C2CCC(=O)NC2=O)C3=O)C1([2H])[2H]] | 3-(6,7-difluoro-1-oxo-4- (piperazin-1-yl-2,2,3,3,5,5,6,6- d₈)isoindolin-2-yl)piperidine-2,6-dione |
| | 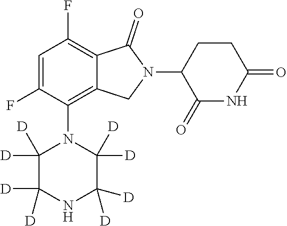 [SMILES: [2H]C1([2H])NC([2H])([2H])C([2H])([2H])N(c2c(F)cc(F)c3c2CN(C2CCC(=O)NC2=O)C3=O)C1([2H])[2H]] | 3-(5,7-difluoro-1-oxo-4- (piperazin-1-yl-2,2,3,3,5,5,6,6- d₈)isoindolin-2-yl)piperidine-2,6-dione |
| | 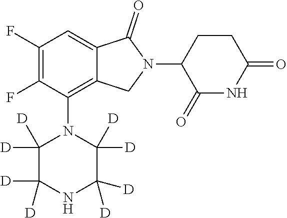 [SMILES: [2H]C1([2H])NC([2H])([2H])C([2H])([2H])N(c2c(F)c(F)cc3c2CN(C2CCC(=O)NC2=O)C3=O)C1([2H])[2H]] | 3-(5,6-difluoro-1-oxo-4- (piperazin-1-yl-2,2,3,3,5,5,6,6- d₈)isoindolin-2-yl)piperidine-2,6-dione |
| | 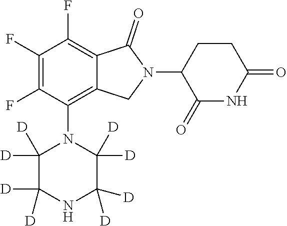 [SMILES: [2H]C1([2H])NC([2H])([2H])C([2H])([2H])N(c2c(F)c(F)c(F)c3c2CN(C2CCC(=O)NC2=O)C3=O)C1([2H])[2H]] | 3-(5,6,7-trifluoro-1-oxo-4- (piperazin-1-yl-2,2,3,3,5,5,6,6- d₈)isoindolin-2-yl)piperidine-2,6-dione |
| | 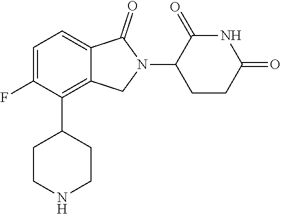 [SMILES: O=C1CCC(N2Cc3c(ccc(F)c3C3CCNCC3)C2=O)C(=O)N1] | 3-(5-fluoro-1-oxo-4- (piperidin-4-yl)isoindolin-2-yl)piperidine- 2,6-dione |
| | 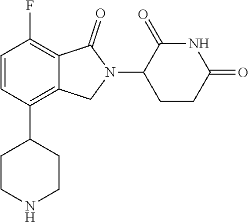 [SMILES: O=C1CCC(N2Cc3c(C4CCNCC4)ccc(F)c3C2=O)C(=O)N1] | 3-(7-fluoro-1-oxo-4- (piperidin-4-yl)isoindolin-2-yl)piperidine- 2,6-dione |
| | 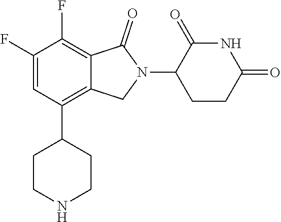 [SMILES: O=C1CCC(N2Cc3c(C4CCNCC4)cc(F)c(F)c3C2=O)C(=O)N1] | 3-(6,7-difluoro-1-oxo-4- (piperidin-4-yl)isoindolin-2- yl)piperidine-2,6-dione |
| | 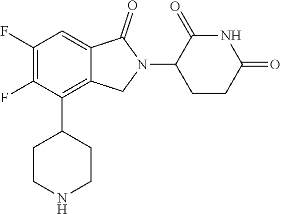 [SMILES: O=C1CCC(N2Cc3c(cc(F)c(F)c3C3CCNCC3)C2=O)C(=O)N1] | 3-(5,6-difluoro-1-oxo-4- (piperidin-4-yl)isoindolin-2- yl)piperidine-2,6-dione |
| | 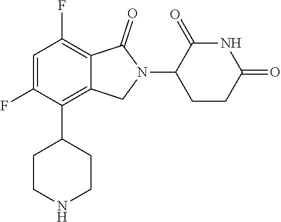 [SMILES: O=C1CCC(N2Cc3c(c(F)cc(F)c3C3CCNCC3)C2=O)C(=O)N1] | 3-(5,7-difluoro-1-oxo-4- (piperidin-4-yl)isoindolin-2- yl)piperidine-2,6-dione |
| | 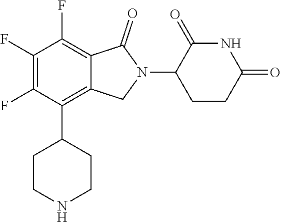 [SMILES: O=C1CCC(N2Cc3c(c(F)c(F)c(F)c3C3CCNCC3)C2=O)C(=O)N1] | 3-(5,6,7-trifluoro-1-oxo-4- (piperidin-4-yl)isoindolin-2- yl)piperidine-2,6-dione |
| | 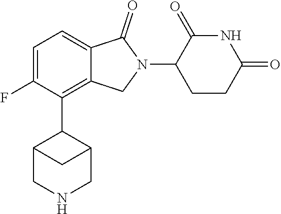 [SMILES: O=C1CCC(N2Cc3c(ccc(F)c3C3C4CNCC3C4)C2=O)C(=O)N1] | 3-(4-(3-azabicyclo[3.1.1]heptan-6-yl)- 5-fluoro-1- oxoisoindolin-2-yl)piperidine-2,6-dione |
| | 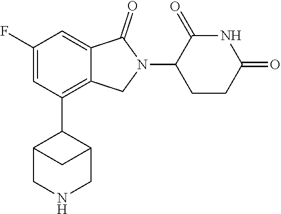 [SMILES: O=C1CCC(N2Cc3c(cc(F)cc3C3C4CNCC3C4)C2=O)C(=O)N1] | 3-(4-(3-azabicyclo[3.1.1]heptan-6-yl)- 6-fluoro-1- oxoisoindolin-2-yl)piperidine-2,6-dione |
| | 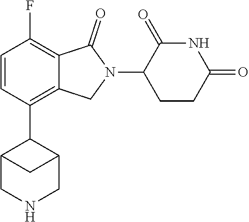 [SMILES: O=C1CCC(N2Cc3c(C4C5CNCC4C5)ccc(F)c3C2=O)C(=O)N1] | 3-(4-(3-azabicyclo[3.1.1]heptan-6-yl)- 7-fluoro-1- oxoisoindolin-2-yl)piperidine-2,6-dione |
| | 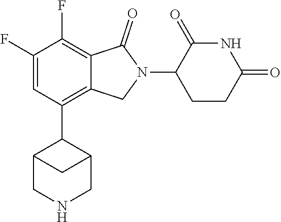 [SMILES: O=C1CCC(N2Cc3c(C4C5CNCC4C5)cc(F)c(F)c3C2=O)C(=O)N1] | 3-(4-(3-azabicyclo[3.1.1]heptan-6-yl)- 6,7-difluoro-1- oxoisoindolin-2-yl)piperidine-2,6-dione |
| | 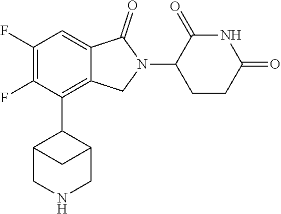 [SMILES: O=C1CCC(N2Cc3c(cc(F)c(F)c3C3C4CNCC3C4)C2=O)C(=O)N1] | 3-(4-(3-azabicyclo[3.1.1]heptan-6-yl)- 5,6-difluoro-1- oxoisoindolin-2-yl)piperidine-2,6-dione |
| | 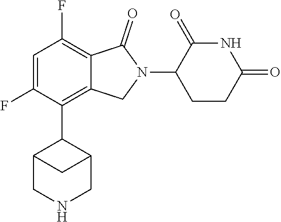 [SMILES: O=C1CCC(N2Cc3c(c(F)cc(F)c3C3C4CNCC3C4)C2=O)C(=O)N1] | 3-(4-(3-azabicyclo[3.1.1]heptan-6-yl)- 5,7-difluoro-1- oxoisoindolin-2-yl)piperidine-2,6-dione |
| | 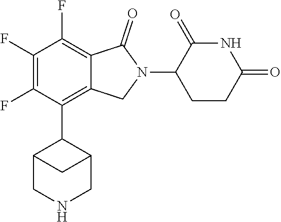 [SMILES: O=C1CCC(N2Cc3c(c(F)c(F)c(F)c3C3C4CNCC3C4)C2=O)C(=O)N1] | 3-(4-(3-azabicyclo[3.1.1]heptan-6-yl)- 5,6,7-trifluoro-1- oxoisoindolin-2-yl)piperidine-2,6-dione |
| | 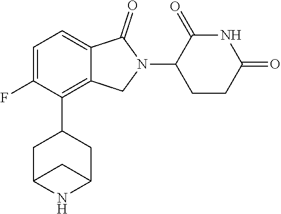 [SMILES: O=C1CCC(N2Cc3c(ccc(F)c3C3CC4CC(C3)N4)C2=O)C(=O)N1] | 3-(4-(6-azabicyclo[3.1.1]heptan-3-yl)- 5-fluoro-1- oxoisoindolin-2-yl)piperidine-2,6-dione |
| | 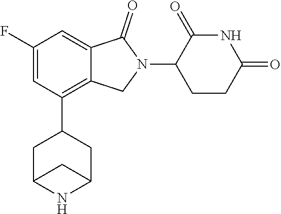 [SMILES: O=C1CCC(N2Cc3c(cc(F)cc3C3CC4CC(C3)N4)C2=O)C(=O)N1] | 3-(4-(6-azabicyclo[3.1.1]heptan-3-yl)- 6-fluoro-1- oxoisoindolin-2-yl)piperidine-2,6-dione |
| | 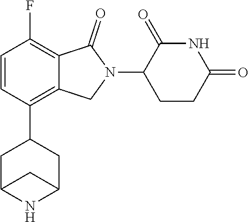 [SMILES: O=C1CCC(N2Cc3c(C4CC5CC(C4)N5)ccc(F)c3C2=O)C(=O)N1] | 3-(4-(6-azabicyclo[3.1.1]heptan-3-yl)- 7-fluoro-1- oxoisoindolin-2-yl)piperidine-2,6-dione |
| | 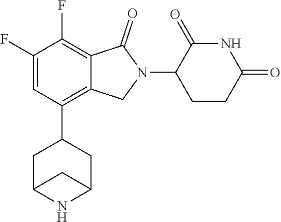 [SMILES: O=C1CCC(N2Cc3c(C4CC5CC(C4)N5)cc(F)c(F)c3C2=O)C(=O)N1] | 3-(4-(6-azabicyclo[3.1.1]heptan-3-yl)- 6,7-difluoro-1- oxoisoindolin-2-yl)piperidine-2,6-dione |
| | 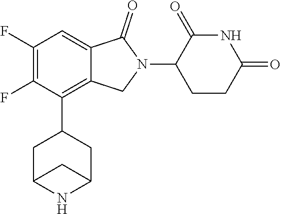 [SMILES: O=C1CCC(N2Cc3c(cc(F)c(F)c3C3CC4CC(C3)N4)C2=O)C(=O)N1] | 3-(4-(6-azabicyclo[3.1.1]heptan-3-yl)- 5,6-difluoro-1- oxoisoindolin-2-yl)piperidine-2,6-dione |
| | 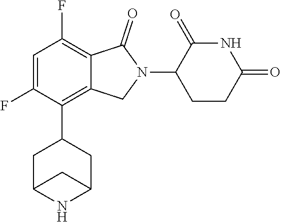 [SMILES: O=C1CCC(N2Cc3c(c(F)cc(F)c3C3CC4CC(C3)N4)C2=O)C(=O)N1] | 3-(4-(6-azabicyclo[3.1.1]heptan-3-yl)- 5,7-difluoro-1- oxoisoindolin-2-yl)piperidine-2,6-dione |
| | 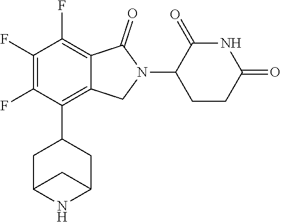 [SMILES: O=C1CCC(N2Cc3c(c(F)c(F)c(F)c3C3CC4CC(C3)N4)C2=O)C(=O)N1] | 3-(4-(6-azabicyclo[3.1.1]heptan-3-yl)- 5,6,7-trifluoro-1- oxoisoindolin-2-yl)piperidine-2,6-dione |
| | 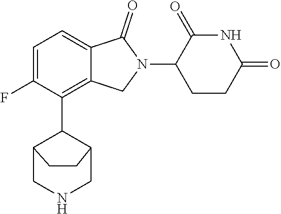 [SMILES: O=C1CCC(N2Cc3c(ccc(F)c3C3C4CCC3CNC4)C2=O)C(=O)N1] | 3-(4-((1R,5S,8r)-3-azabicyclo[3.2.1]octan-8-yl)- 5-fluoro-1- oxoisoindolin-2-yl)piperidine-2,6-dione |
| | 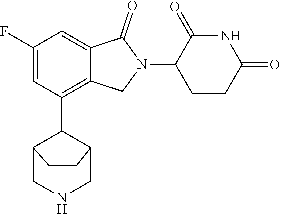 [SMILES: O=C1CCC(N2Cc3c(cc(F)cc3C3C4CCC3CNC4)C2=O)C(=O)N1] | 3-(4-((1R,5S,8r)-3-azabicyclo[3.2.1]octan-8-yl)- 6-fluoro-1- oxoisoindolin-2-yl)piperidine-2,6-dione |
| | 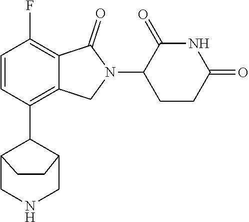 [SMILES: O=C1CCC(N2Cc3c(C4C5CCC4CNC5)ccc(F)c3C2=O)C(=O)N1] | 3-(4-((1R,5S,8r)-3-azabicyclo[3.2.1]octan-8-yl)- 7-difluoro- 1-oxoisoindolin-2-yl)piperidine-2,6-dione |
| | 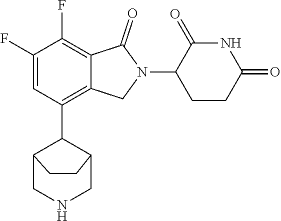 [SMILES: O=C1CCC(N2Cc3c(C4C5CCC4CNC5)cc(F)c(F)c3C2=O)C(=O)N1] | 3-(4-((1R,5S,8r)-3-azabicyclo[3.2.1]octan-8-yl)- 6,7-difluoro- 1-oxoisoindolin-2-yl)piperidine-2,6-dione |
| | 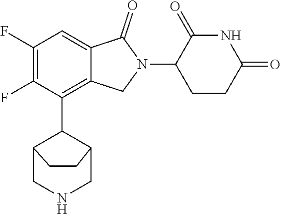 [SMILES: O=C1CCC(N2Cc3c(cc(F)c(F)c3C3C4CCC3CNC4)C2=O)C(=O)N1] | 3-(4-((1R,5S,8r)-3-azabicyclo[3.2.1]octan-8-yl)- 5,6-difluoro 1-oxoisoindolin-2-yl)piperidine-2,6-dione |
| | 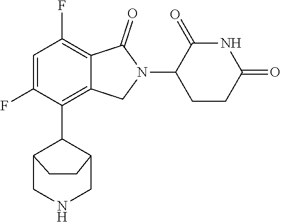 [SMILES: O=C1CCC(N2Cc3c(c(F)cc(F)c3C3C4CCC3CNC4)C2=O)C(=O)N1] | 3-(4-((1R,5S,8r)-3-azabicyclo[3.2.1]octan-8-yl)- 5,7-difluoro- 1-oxoisoindolin-2-yl)piperidine-2,6-dione |
| | 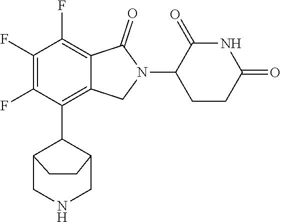 [SMILES: O=C1CCC(N2Cc3c(c(F)c(F)c(F)c3C3C4CCC3CNC4)C2=O)C(=O)N1] | 3-(4-((1R,5S,8r)-3-azabicyclo[3.2.1]octan-8-yl)- 5,6,7-trifluoro-1-oxoisoindolin-2-yl)piperidine- 2,6-dione |
| | 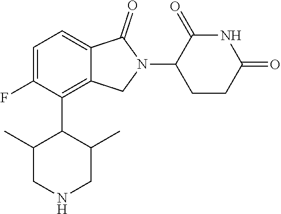 [SMILES: CC1CNCC(C)C1c1c(F)ccc2c1CN(C1CCC(=O)NC1=O)C2=O] | 3-(4-(3,5-dimethylpiperidin-4-yl)- 5-fluoro-1-oxoisoindolin-2- yl)piperidine-2,6-dione |
| | 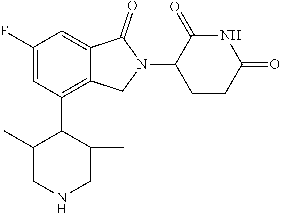 [SMILES: CC1CNCC(C)C1c1cc(F)cc2c1CN(C1CCC(=O)NC1=O)C2=O] | 3-(4-(3,5-dimethylpiperidin-4-yl)- 6-fluoro-1-oxoisoindolin-2- yl)piperidine-2,6-dione |
| | 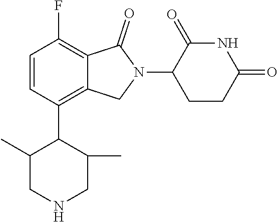 [SMILES: CC1CNCC(C)C1c1ccc(F)c2c1CN(C1CCC(=O)NC1=O)C2=O] | 3-(4-(3,5-dimethylpiperidin-4-yl)- 7-fluoro-1-oxoisoindolin-2- yl)piperidine-2,6-dione |
| | 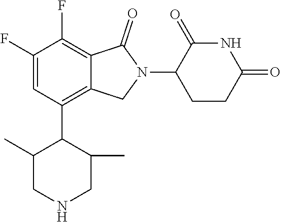 [SMILES: CC1CNCC(C)C1c1cc(F)c(F)c2c1CN(C1CCC(=O)NC1=O)C2=O] | 3-(4-(3,5-dimethylpiperidin-4-yl)- 6,7-difluoro-1- oxoisoindolin-2-yl)piperidine-2,6-dione |
| | 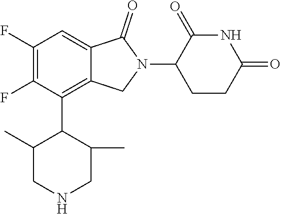 [SMILES: CC1CNCC(C)C1c1c(F)c(F)cc2c1CN(C1CCC(=O)NC1=O)C2=O] | 3-(4-(3,5-dimethylpiperidin-4-yl)- 5,6-difluoro-1- oxoisoindolin-2-yl)piperidine-2,6-dione |
| | 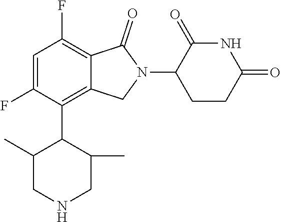 [SMILES: CC1CNCC(C)C1c1c(F)cc(F)c2c1CN(C1CCC(=O)NC1=O)C2=O] | 3-(4-(3,5-dimethylpiperidin-4-yl)- 5,7-difluoro-1- oxoisoindolin-2-yl)piperidine-2,6-dione |
| | 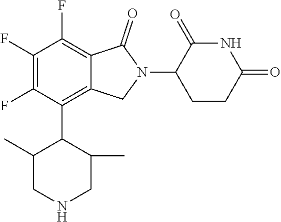 [SMILES: CC1CNCC(C)C1c1c(F)c(F)c(F)c2c1CN(C1CCC(=O)NC1=O)C2=O] | 3-(4-(3,5-dimethylpiperidin-4-yl)- 5,6,7-trifluoro-1- oxoisoindolin-2-yl)piperidine-2,6-dione |

### Compounds of Formula (II)

The present disclosure provides a compound of Formula (II) or salts (including pharmaceutically acceptable salts), stereoisomers (including enantiomers), solvates, isotopically enriched analogs, prodrugs, or polymorphs thereof:

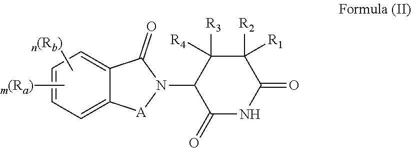

wherein A, R₁, R₂, R₃, R₄, (R_{b})ₙ and (Rₐ)ₘ are as defined in the compound of Formula (II) above.

In some embodiments, R₁, R₂, R₃ and R₄ of the compound of Formula (II) are the same or different and each independently represent hydrogen, deuterium, halogen (e.g., fluorine, chlorine, bromine, or iodine), optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—).

In some embodiments, R₁, R₂, R₃ and R₄ of the compound of Formula (II) are the same or different and each independently represent hydrogen or deuterium.

In some embodiments, R₁, R₂, R₃ and R₄ of the compound of Formula (II) represents hydrogen.

In some embodiments, A of the compound of Formula (II) represents CO.

In some embodiments, A of the compound of Formula (II) represents CH₂.

In some embodiments, A of the compound of Formula (II) represents CD₂.

In some embodiments, m of the compound of Formula (II) represents an integer of 1, n represents an integer of 0, 1, 2, or 3.

In some embodiments, m of the compound of Formula (II) represents an integer of 2, n represents an integer of 0, 1 or 2.

In some embodiments, in case that the compound of Formula (II) is partially or fully deuterated, when W represents optionally substituted nitrogen-containing monocyclic heterocyclyl, optionally substituted nitrogen-containing bridged heterocyclyl, optionally substituted cycloalkyl, optionally substituted heteroaryl or optionally substituted aryl, LIN represents a bond, optionally substituted methylene, or optionally substituted linear or branched C₂₋₃₀ alkylene, wherein one or more (e.g., 1-10, 1-8, or 1-6, such as 1, 2, 3, 4, 5 or 6) groups Rₑ and/or one or more (e.g., 1-10, 1-8, or 1-6, such as 1, 2, 3, 4, 5 or 6) groups R_{f} and/or any combination of one or more (e.g., 1-10, 1-8, or 1-6, such as 1, 2, 3, 4, 5 or 6) groups Rₑ and R_{f} are optionally inserted into the backbone carbon chain of the linear or branched C₂₋₃₀ alkylene group, wherein carbon-carbon bond between one or more (e.g., 1-10, 1-8, or 1-6, such as 1, 2, 3, 4, 5 or 6) pairs of adjacent carbon atoms in the backbone carbon chain is interrupted by the group Rₑ, R_{f}, or a combination of Rₑ and R_{f}; wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl (e.g., C₁₋₄ alkyl, such as methyl, ethyl, propyl, isopropyl, butyl, sec-butyl or tert-butyl), and in case that two or more groups Rₑ are inserted into the backbone carbon chain of the linear or branched C₂₋₃₀ alkylene group, the two or more groups Rₑ are not directly connected to each other; and wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), hydroxy, amino, mercapto, halogen (e.g., fluorine, chlorine, bromine, or iodine), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—), cyano or any combination thereof.

In some embodiments, in case that the compound of Formula (II) is partially deuterated (for example, carrying 1 to 20 deuterium atoms) or fully deuterated, when W represents optionally substituted nitrogen-containing monocyclic heterocyclyl, optionally substituted nitrogen-containing bridged heterocyclyl, optionally substituted cycloalkyl, optionally substituted heteroaryl or optionally substituted aryl, LIN represents a bond, optionally substituted methylene, or optionally substituted linear or branched C₂₋₃₀ alkylene, wherein one or more (e.g., 1-10, 1-8, or 1-6, such as 1, 2, 3, 4, 5 or 6) groups Rₑ and/or one or more (e.g., 1-10, 1-8, or 1-6, such as 1, 2, 3, 4, 5 or 6) groups R_{f} and/or any combination of one or more (e.g., 1-10, 1-8, or 1-6, such as 1, 2, 3, 4, 5 or 6) groups Rₑ and R_{f} are optionally inserted into the backbone carbon chain of the linear or branched C₂₋₃₀ alkylene group, wherein carbon-carbon bond between one or more (e.g., 1-10, 1-8, or 1-6, such as 1, 2, 3, 4, 5 or 6) pairs of adjacent carbon atoms in the backbone carbon chain is interrupted by the group Rₑ, R_{f}, or a combination of Rₑ and R_{f}; wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl (e.g., C₁₋₄ alkyl, such as methyl, ethyl, propyl, isopropyl, butyl, sec-butyl or tert-butyl), and in case that two or more groups Rₑ are inserted into the backbone carbon chain of the linear or branched C₂₋₃₀ alkylene group, the two or more groups Rₑ are not directly connected to each other; and wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), hydroxy, amino, mercapto, halogen (e.g., fluorine, chlorine, bromine, or iodine), hydroxy, amino, mercapto, halogen, optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—), cyano or any combination thereof;
wherein the methylene and the linear or branched C₂₋₃₀ alkylene are optionally substituted with one or more (e.g., 1-10, 1-8, or 1-6, such as 1, 2, 3, 4, 5 or 6) substituents selected from the group consisting of deuterium, hydroxy, amino, mercapto, nitro, halogen (e.g., fluorine, chlorine, bromine, or iodine), cyano, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—) or any combination thereof.

In some embodiments, in case that the compound of Formula (II) is partially or fully deuterated, when W represents hydrogen, C₂₋₆ alkenyl, C₂₋₆ alkynyl, a leaving group, CHO, COOH, NH₂, hydroxy or mercapto, LIN represents optionally substituted methylene or optionally substituted linear or branched C₂₋₃₀ alkylene, wherein one or more (e.g., 1-10, 1-8, or 1-6, such as 1, 2, 3, 4, 5 or 6) groups Rₑ and/or one or more (e.g., 1-10, 1-8, or 1-6, such as 1, 2, 3, 4, 5 or 6) groups R_{f} and/or any combination of one or more (e.g., 1-10, 1-8, or 1-6, such as 1, 2, 3, 4, 5 or 6) groups Rₑ and R_{f} are optionally inserted into the backbone carbon chain of the linear or branched C₂₋₃₀ alkylene group, wherein carbon-carbon bond between one or more (e.g., 1-10, 1-8, or 1-6, such as 1, 2, 3, 4, 5 or 6) pairs of adjacent carbon atoms in the backbone carbon chain is interrupted by the group Rₑ, R_{f}, or a combination of Rₑ and R_{f}; each Rₑ are selected from the group consisting of O, N(R_{g}), C(O), C(O)O, S(O), S(O)₂, S(O)₂NH, NHS(O)₂, OC(O), C(O)N(R_{g}), N(R_{g})C(O), or N(R_{g})C(O)N(R_{g}), where each R_{g} independently represent H or C₁₋₆ alkyl (e.g., C₁₋₄ alkyl, such as methyl, ethyl, propyl, isopropyl, butyl, sec-butyl or tert-butyl), and in case that two or more groups Rₑ are inserted into the backbone carbon chain of the linear or branched C₂₋₃₀ alkylene group, the two or more groups Rₑ are not directly connected to each other; and each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), hydroxyl, amino, mercapto, halogen (e.g., fluorine, chlorine, bromine, or iodine), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—), cyano or any combination thereof.

In some embodiments, in case that the compound of Formula (II) is partially deuterated (for example, carrying 1 to 20 deuterium atoms) or fully deuterated, when W represents hydrogen, C₂₋₆ alkenyl, C₂₋₆ alkynyl, a leaving group, CHO, COOH, NH₂, hydroxy or mercapto, LIN represents optionally substituted methylene or optionally substituted linear or branched C₂₋₃₀ alkylene, wherein one or more (e.g., 1-10, 1-8, or 1-6, such as 1, 2, 3, 4, 5 or 6) groups Rₑ and/or one or more (e.g., 1-10, 1-8, or 1-6, such as 1, 2, 3, 4, 5 or 6) groups R_{f} and/or any combination of one or more (e.g., 1-10, 1-8, or 1-6, such as 1, 2, 3, 4, 5 or 6) groups Rₑ and R_{f} are optionally inserted into the backbone carbon chain of the linear or branched C₂₋₃₀ alkylene group, wherein carbon-carbon bond between one or more (e.g., 1-10, 1-8, or 1-6, such as 1, 2, 3, 4, 5 or 6) pairs of adjacent carbon atoms in the backbone carbon chain is interrupted by the group Rₑ, R_{f}, or a combination of Rₑ and R_{f}; each Rₑ are selected from the group consisting of O, N(R_{g}), C(O), C(O)O, S(O), S(O)₂, S(O)₂NH, NHS(O)₂, OC(O), C(O)N(R_{g}), N(R_{g})C(O), or N(R_{g})C(O)N(R_{g}), where each R_{g} independently represent H or C₁₋₆ alkyl (e.g., C₁₋₄ alkyl, such as methyl, ethyl, propyl, isopropyl, butyl, sec-butyl or tert-butyl), and in case that two or more groups Rₑ are inserted into the backbone carbon chain of the linear or branched C₂₋₃₀ alkylene group, the two or more groups Rₑ are not directly connected to each other; and each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), hydroxyl, amino, mercapto, halogen (e.g., fluorine, chlorine, bromine, or iodine), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—), cyano or any combination thereof;
wherein the methylene and the linear or branched C₂₋₃₀ alkylene are optionally substituted with one or more (e.g., 1-10, 1-8, or 1-6, such as 1, 2, 3, 4, 5 or 6) substituents selected from the group consisting of deuterium, hydroxy, amino, mercapto, nitro, halogen (e.g., fluorine, chlorine, bromine, or iodine), cyano, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—) or any combination thereof.

In some embodiments, in case that the compound of Formula (II) is not deuterated, LIN represents optionally substituted methylene, and W represents optionally substituted nitrogen-containing monocyclic heterocyclyl or optionally substituted nitrogen-containing bridged heterocyclyl;
wherein the methylene is optionally substituted with one or more (e.g., 1 or 2) substituents selected from the group consisting of deuterium, hydroxy, amino, mercapto, nitro, halogen (e.g., fluorine, chlorine, bromine, or iodine), cyano, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—) or any combination thereof.

In some embodiments, in case that the compound of Formula (II) is not deuterated, when LIN represents optionally substituted methylene, R_{c} represents piperazinylene, and W represents optionally substituted nitrogen-containing monocyclic heterocyclyl, the piperazinylene is substituted. In some embodiments, the piperazinylene of R_{c} is substituted with 1-4 (e.g., 1-3 or 1-2, such as 1, 2, 3 or 4) substituents selected from the group consisting of deuterium, hydroxy, amino, mercapto, nitro, halogen (e.g., fluorine, chlorine, bromine, or iodine), cyano, CHO, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—) or any combination thereof. In some embodiments, the methylene of LIN is optionally substituted with one or more (e.g., 1 or 2) substituents selected from the group consisting of deuterium, hydroxy, amino, mercapto, nitro, halogen (e.g., fluorine, chlorine, bromine, or iodine), cyano, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—) or any combination thereof. In some embodiments, the nitrogen-containing monocyclic heterocyclyl of W is optionally substituted with one or more (e.g., 1-10, 1-8, or 1-6, such as 1, 2, 3, 4, 5 or 6) substituents selected from the group consisting of deuterium, hydroxy, amino, mercapto, nitro, halogen (e.g., fluorine, chlorine, bromine, or iodine), cyano, CHO, COOH, N₃, methanesulfonyloxy, trifluoromethanesulfonyloxy, p-toluenesulfonyloxy, C₁₋₆ alkyl, deuterated C₁₋₆ alkyl, halogenated C₁₋₆ alkyl, optionally substituted C₃-8 cycloalkyl (e.g., substituted with one or more, e.g., 1-6, such as 1, 2, 3, 4, 5 or 6 substituents selected from the group consisting of deuterium, halogen, amino, hydroxy, mercapto, cyano, CHO, COOH, N₃, methanesulfonyloxy, trifluoromethanesulfonyloxy and p-toluenesulfonyloxy), optionally substituted 4- to 9-membered heterocyclyl (e.g., substituted with one or more, e.g., 1-6, such as 1, 2, 3, 4, 5 or 6 substituents selected from the group consisting of deuterium, halogen, amino, hydroxy, mercapto, cyano, CHO, COOH, N₃, methanesulfonyloxy, trifluoromethanesulfonyloxy and p-toluenesulfonyloxy), C₁₋₆ alkoxy (e.g., C₁₋₄ alkoxy, such as methoxy, CD₃-O—, ethoxy, propoxy, isopropoxy, butoxy, sec-butoxy or tert-butoxy), deuterated C₁₋₆ alkyl-O— (e.g., perdeuterated C₁₋₄ alkoxy, such as CD₃-O—, perdeuterated ethoxy, perdeuterated propoxy, perdeuterated isopropoxy, perdeuterated butoxy, perdeuterated sec-butoxy or perdeuterated tert-butoxy), C₁₋₆ alkyl-NH— (e.g., C₁₋₃ alkyl-NH—, such as CH₃NH—, CH₃CH₂NH— or CH₃CH₂CH₂NH—), deuterated C₁₋₆ alkyl-NH— (e.g., perdeuterated C₁₋₃ alkyl-NH—, such as CD₃NH—, CD₃CD₂NH— or CD₃CD₂CD₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), C₁₋₆ alkyl-NHC(O)— (e.g., C₁₋₄ alkyl-NHC(O)—, such as CH₃—NHC(O)—, CH₃CH₂—NHC(O)—, and CH₃CH₂CH₂—NHC(O)—), deuterated C₁₋₆ alkyl-NHC(O)— (e.g., perdeuterated C₁₋₄ alkyl-NHC(O)—, such as CD₃-NHC(O)—, CD₃CD₂-NHC(O)—, and CD₃CD₂CD₂-NHC(O)—), C₁₋₆ alkyl-C(O)NH— (e.g., C₁₋₄ alkyl-C(O)NH—, such as CH₃—C(O)NH—, CH₃CH₂—C(O)NH—, and CH₃CH₂CH₂—C(O)NH—), deuterated C₁₋₆ alkyl-C(O)NH— (e.g., perdeuterated C₁₋₄ alkyl-C(O)NH—, such as CD₃-C(O)NH—, CD₃CD₂-C(O)NH—, and CD₃CD₂CD₂-C(O)NH—) or any combination thereof.

In some embodiments, R_{c} of the compound of Formula (II) represents the following groups:
monocyclic C₃₋₁₀ heterocyclylene group containing one nitrogen atom and optionally a heteroatom selected from nitrogen, oxygen, and sulfur, e.g., piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene or diazacyclooctylene; or
C₅₋₂₀ bridged heterocyclylene group containing one nitrogen atom and optionally a heteroatom selected from nitrogen, oxygen, and sulfur, e.g., azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene or diazabicyclo[2.2.2]octanylene, such as the following groups:

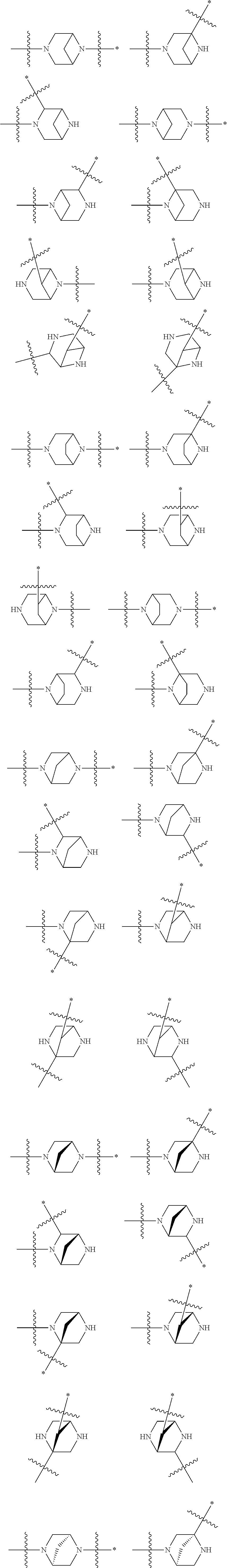

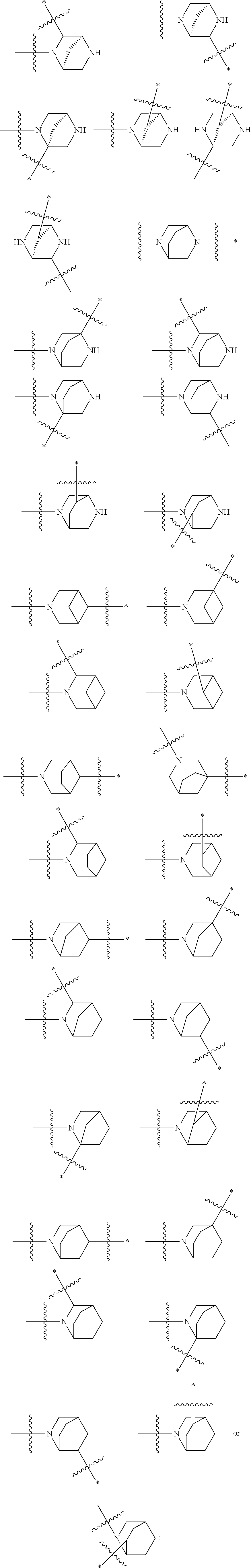

wherein symbol * indicates the point of attachment to U₁,
wherein the monocyclic C₃₋₁₀ heterocyclylene and the C₅₋₂₀ bridged heterocyclylene group are each independently optionally substituted with one or more (e.g., 1-10, 1-8 or 1-6, such as 1, 2, 3, 4, 5 or 6) substituents selected from the group consisting of deuterium, hydroxy, amino, mercapto, nitro, halogen (e.g., fluorine, chlorine, bromine, or iodine), cyano, CHO, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—) or any combination thereof.

In some embodiments, R_{c} of the compound of Formula (II) represents the following groups:

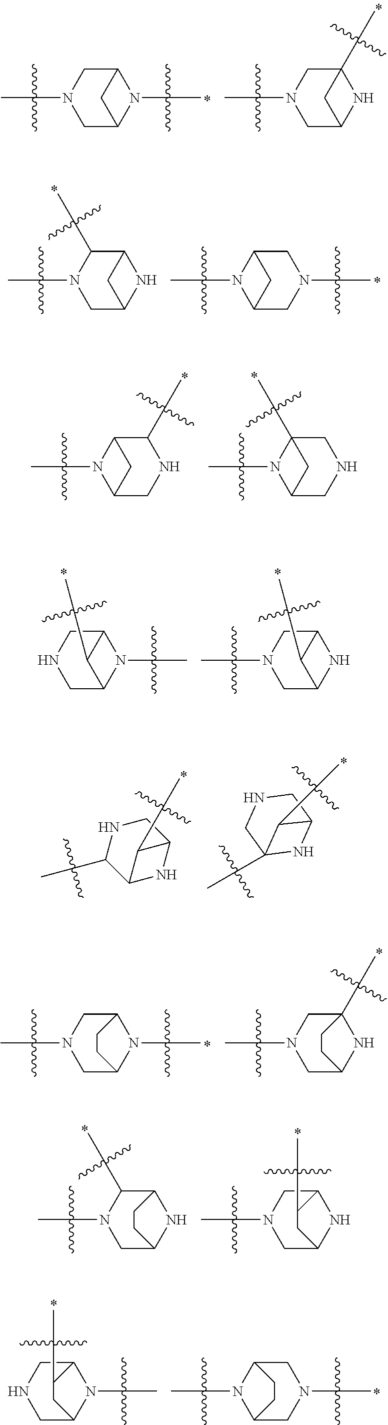

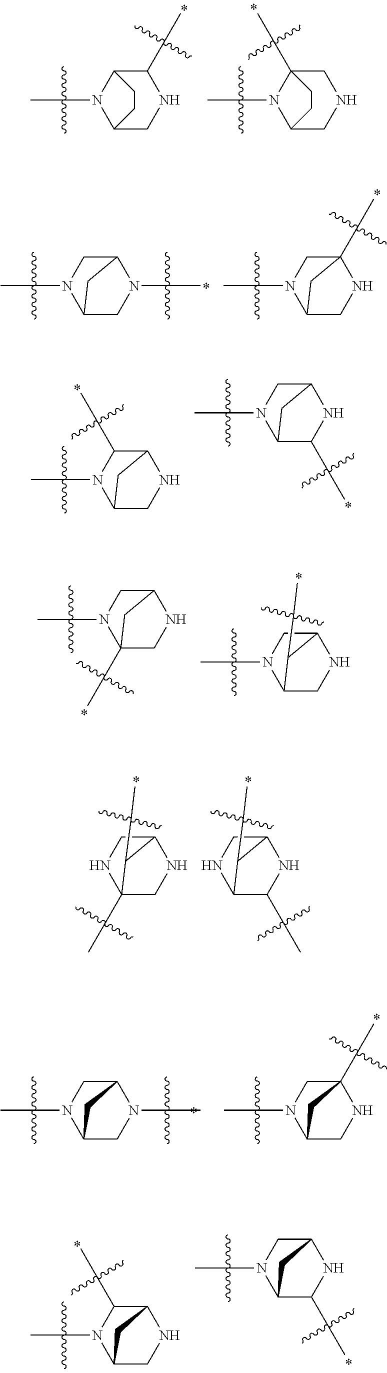

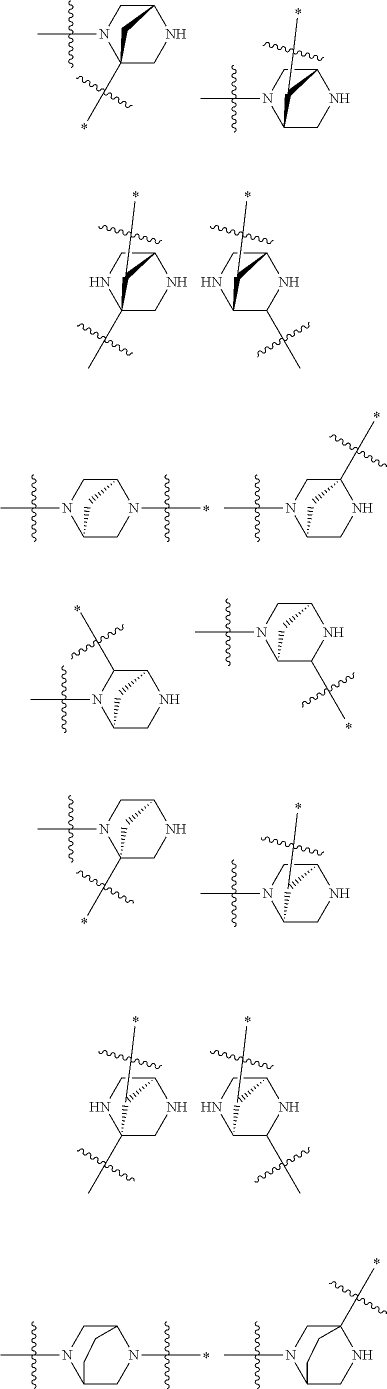

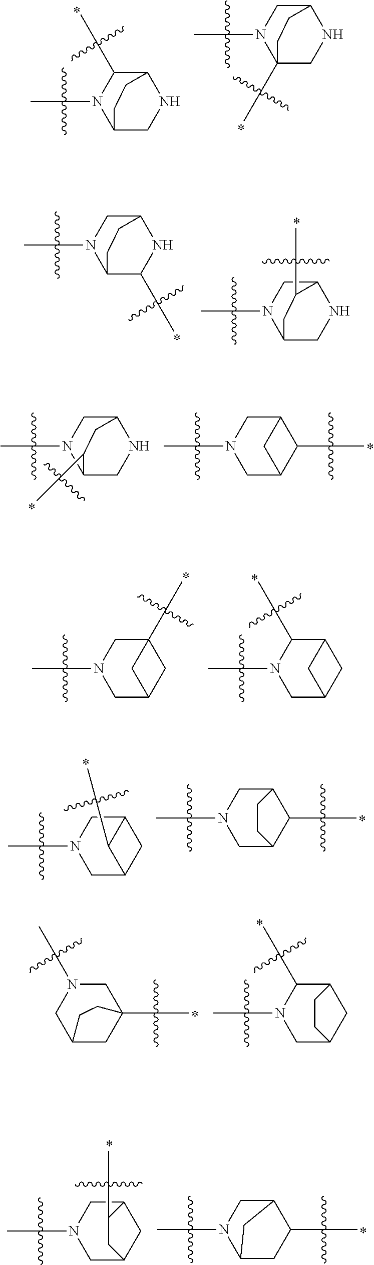

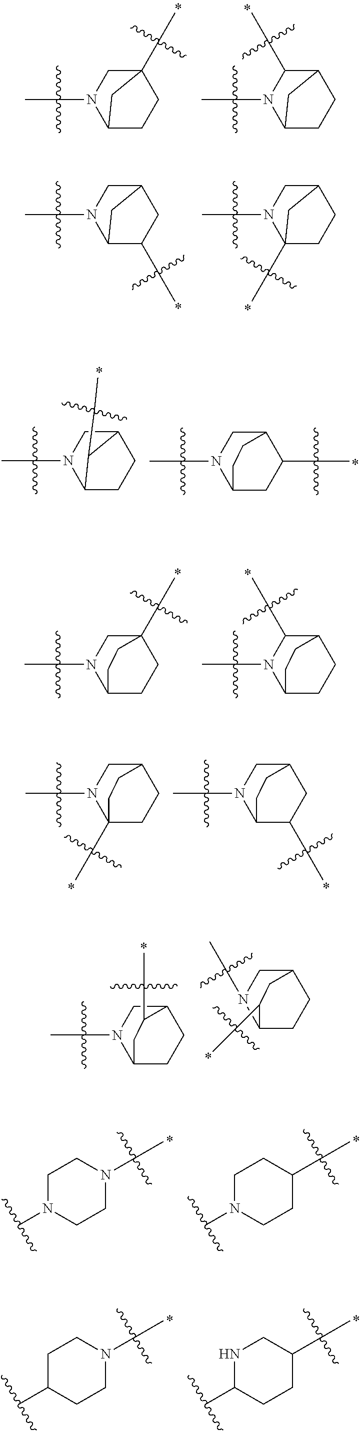

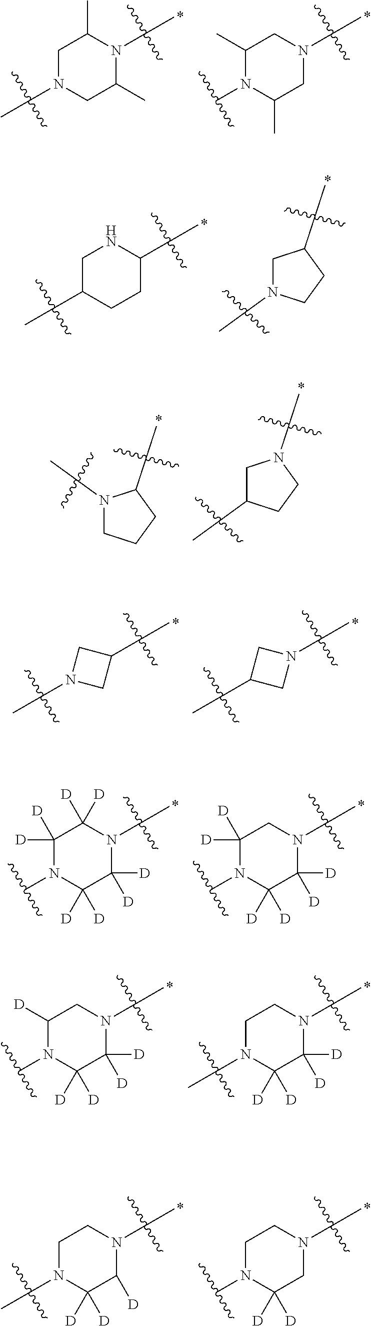

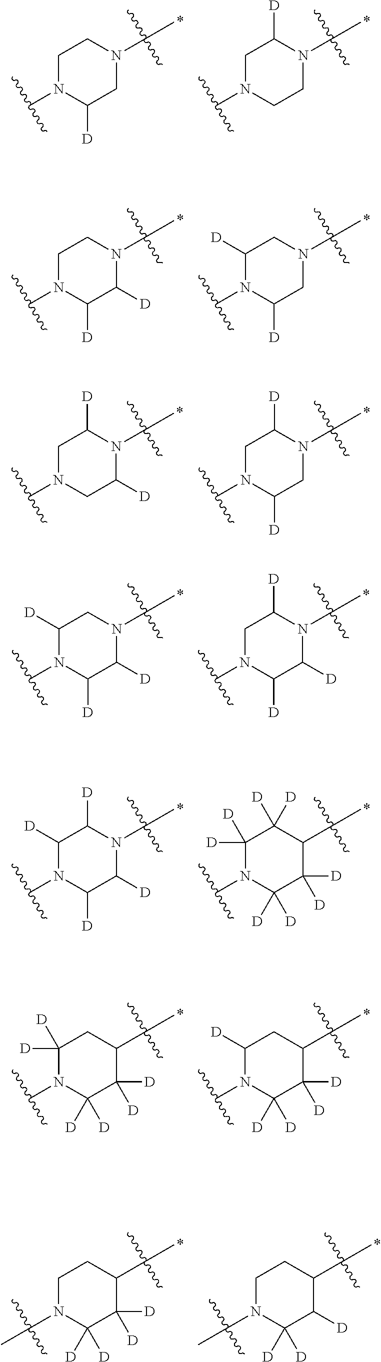

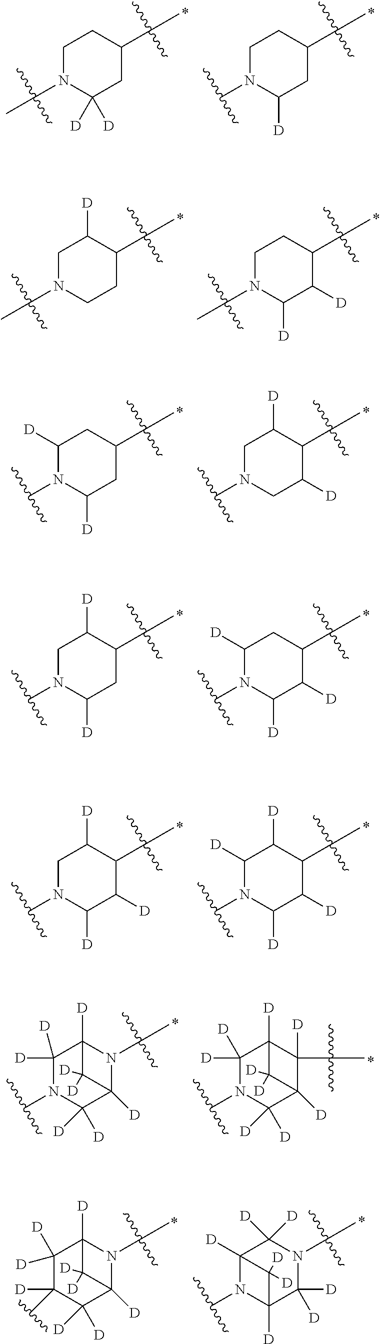

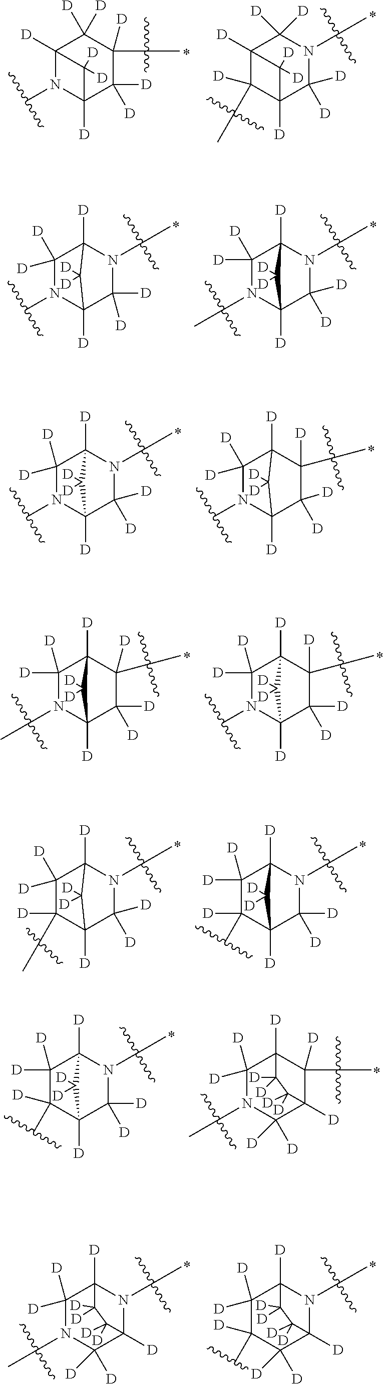

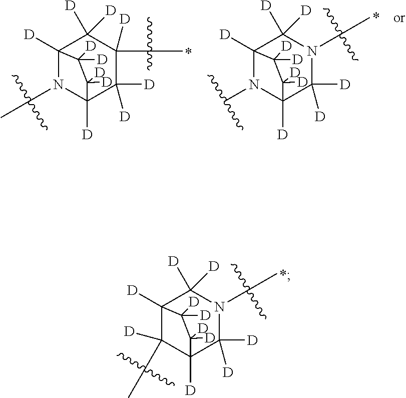 [SMILES: *N1C2([2H])C([2H])([2H])C(*)([2H])C([2H])([2H])C1([2H])C([2H])([2H])C2([2H])[2H] ; *N1C([2H])([2H])C2([2H])N(*)C([2H])(C1([2H])[2H])C([2H])([2H])C2([2H])[2H] ; *N1C([2H])([2H])C2([2H])C(*)([2H])C([2H])(C1([2H])[2H])C([2H])([2H])C2([2H])[2H]]

wherein symbol * indicates the point of attachment to U₁.

In some embodiments, W of the compound of Formula (II) represents the following groups:
monocyclic C₃₋₁₀ heterocyclyl group containing one nitrogen atom and optionally a heteroatom selected from nitrogen, oxygen, and sulfur, e.g., piperidinyl, piperazinyl, morpholinyl, azetidinyl, pyrrolidinyl, imidazolidyl, pyrazolidyl, oxazolidinyl, thiazolidinyl, thiomorpholinyl, azepanyl, diazacycloheptanyl, azacyclooctyl or diazacyclooctyl; or
C₅₋₂₀ bridged heterocyclyl group containing one nitrogen atom and optionally a heteroatom selected from nitrogen, oxygen, and sulfur, e.g., azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl or diazabicyclo[2.2.2]octanyl, such as the following groups:

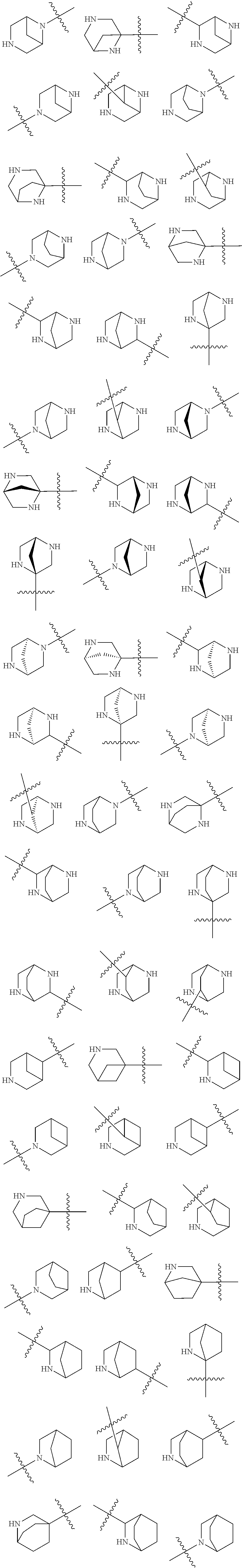

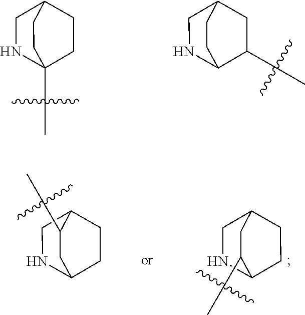 [SMILES: *C12CCC(CC1)CN2 ; *C1CC2CCC1NC2 ; *C1CC2CCC1CN2 ; "or" ; *C1CC2CCC1NC2 ; ";"]

or
saturated or partially unsaturated monocyclic or bicyclic or polycyclic C₃₋₂₀ cycloalkyl, e.g., cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, C₇₋₁₁ spiro-cycloalkyl, adamantanyl, noradamantanyl, bornyl, bicyclo[2.2.1]heptyl or bicyclo[2.2.1]heptenyl; or
heteroaryl, e.g., 5-to 20-membered monocyclic or bicyclic aromatic ring group containing one or more (e.g., 1-4, such as 1, 2, 3 or 4) heteroatoms independently selected from the group consisting of oxygen, nitrogen, and sulfur, e.g., furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-a]pyrimidinyl, imidazo[1,2-a]pyridyl, 1H-pyrrolo[3,2-b]pyridyl, 1H-pyrrolo[2,3-b]pyridyl, 4H-fluoro[3,2-b]pyrrolyl, pyrrolo[2,1-b]thiazolyl or imidazo[2,1-b]thiazolyl; or
C₅₋₁₄ aryl optionally containing one or more (e.g., 1-3, such as 1, 2 or 3) fused rings, e.g., phenyl, naphthyl or fluorenyl; and
wherein the monocyclic C₃₋₁₀ heterocyclyl, C₅₋₂₀ bridged heterocyclyl, C₃₋₂₀ cycloalkyl, heteroaryl and C₅₋₁₄ aryl are each independently optionally substituted with one or more (e.g., 1-10, 1-8, or 1-6, such as 1, 2, 3, 4, 5 or 6) substituents selected from the group consisting of deuterium, hydroxy, amino, mercapto, nitro, halogen (e.g., fluorine, chlorine, bromine, or iodine), cyano, CHO, COOH, N₃, methanesulfonyloxy, trifluoromethanesulfonyloxy, p-toluenesulfonyloxy, C₁₋₆ alkyl, deuterated C₁₋₆ alkyl, halogenated C₁₋₆ alkyl, optionally substituted C₃₋₈ cycloalkyl (e.g., substituted with one or more, e.g., 1-6, such as 1, 2, 3, 4, 5 or 6 substituents selected from the group consisting of deuterium, halogen, amino, hydroxy, mercapto, cyano, CHO, COOH, N₃, methanesulfonyloxy, trifluoromethanesulfonyloxy and p-toluenesulfonyloxy), optionally substituted 4- to 9-membered heterocyclyl (e.g., substituted with one or more, e.g., 1-6, such as 1, 2, 3, 4, 5 or 6 substituents selected from the group consisting of deuterium, halogen, amino, hydroxy, mercapto, cyano, CHO, COOH, N₃, methanesulfonyloxy, trifluoromethanesulfonyloxy and p-toluenesulfonyloxy), C₁₋₆ alkoxy (e.g., C₁₋₄ alkoxy, such as methoxy, CD₃-O—, ethoxy, propoxy, isopropoxy, butoxy, sec-butoxy or tert-butoxy), deuterated C₁₋₆ alkyl-O— (e.g., perdeuterated C₁₋₄ alkoxy, such as CD₃-O—, perdeuterated ethoxy, perdeuterated propoxy, perdeuterated isopropoxy, perdeuterated butoxy, perdeuterated sec-butoxy or perdeuterated tert-butoxy), C₁₋₆ alkyl-NH— (e.g., C₁₋₃ alkyl-NH—, such as CH₃NH—, CH₃CH₂NH— or CH₃CH₂CH₂NH—), deuterated C₁₋₆ alkyl-NH— (e.g., perdeuterated C₁₋₃ alkyl-NH—, such as CD₃NH—, CD₃CD₂NH— or CD₃CD₂CD₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), C₁₋₆ alkyl-NHC(O)— (e.g., C₁₋₄ alkyl-NHC(O)—, such as CH₃—NHC(O)—, CH₃CH₂—NHC(O)—, and CH₃CH₂CH₂—NHC(O)—), deuterated C₁₋₆ alkyl-NHC(O)— (e.g., perdeuterated C₁₋₄ alkyl-NHC(O)—, such as CD₃-NHC(O)—, CD₃CD₂-NHC(O)—, and CD₃CD₂CD₂-NHC(O)—), C₁₋₆ alkyl-C(O)NH— (e.g., C₁₋₄ alkyl-C(O)NH—, such as CH₃—C(O)NH—, CH₃CH₂—C(O)NH—, and CH₃CH₂CH₂—C(O)NH—), deuterated C₁₋₆ alkyl-C(O)NH— (e.g., perdeuterated C₁₋₄ alkyl-C(O)NH—, such as CD₃-C(O)NH—, CD₃CD₂-C(O)NH—, and CD₃CD₂CD₂-C(O)NH—) or any combination thereof.

In some embodiments, LIN of the compound of Formula (II) represents the structure of the following formula:
#-C₁₋₃₀ alkylene-;
#-(C(R^{a1})(R^{a2}))ₙ₁—(Rₑ(C(R^{a3})(R^{a4}))ₙ₂)ₘ₁—;
#-(C(R^{a1})(R^{a2}))ₙ₁—(Rₑ(C(R^{a3})(R^{a4}))ₙ₂)ₘ₁—(Rₑ(C(R^{a5})(R^{a6}))ₙ₃)ₘ₂—;
#-(C(R^{a1})(R^{a2}))ₙ₁—(Rₑ(C(R^{a3})(R^{a4}))ₙ₂)ₘ₁—(Rₑ(C(R^{a5})(R^{a6}))ₙ₃)ₘ₂—(Rₑ(C(R^{a7})(R^{a8}))ₙ₄)ₘ₃—;
#-(C(R^{a1})(R^{a2}))ₙ₁—(R_{f}(C(R^{a3})(R^{a4}))ₙ₂)ₘ₁—;
#-(C(R^{a1})(R^{a2}))ₙ₁—(R_{f}(C(R^{a3})(R^{a4}))ₙ₂)ₘ₁—(R_{f}(C(R^{a5})(R^{a6}))ₙ₃)ₘ₂—;
#-(C(R^{a1})(R^{a2}))ₙ₁—(R_{f}(C(R^{a3})(R^{a4}))ₙ₂)ₘ₁—(R_{f}(C(R^{a5})(R^{a6}))ₙ₃)ₘ₂—(R_{f}(C(R^{a7})(R^{a8}))ₙ₄)ₘ₃—;
#-(C(R^{a1})(R^{a2}))ₙ₁—(Rₑ—R_{f}—(C(R^{a3})(R^{a4}))ₙ₂)ₘ₁—;
#-(C(R^{a1})(R^{a2}))ₙ₁—(Rₑ(C(R^{a3})(R^{a4}))ₙ₂)ₘ₁—(R_{f}(C(R^{a5})(R^{a6}))ₙ₃)ₘ₂—;
#-(C(R^{a1})(R^{a2}))ₙ₁—(R_{f}—Rₑ—(C(R^{a3})(R^{a4}))ₙ₂)ₘ₁—; or
#-(C(R^{a1})(R^{a2}))ₙ₁—(R_{f}(C(R^{a3})(R^{a4}))ₙ₂)ₘ₁—(Rₑ(C(R^{a5})(R^{a6}))ₙ₃)ₘ₂—;
wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl;
wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), hydroxy, amino, mercapto, halogen (e.g., fluorine, chlorine, bromine, or iodine), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—), cyano, or any combination thereof;
a hydrogen atom of one or more (e.g., 1-30) CH₂ groups of the C₁₋₃₀ alkylene group is optionally replaced with a substituent selected from the group consisting of deuterium, hydroxy, amino, mercapto, nitro, halogen (e.g., fluorine, chlorine, bromine, or iodine), cyano, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—), or any combination thereof;
R^{a1}, R^{a2}, R^{a3}, R^{a4}, R^{a5}, R^{a6}, R^{a7}, and R^{a8} each independently represent H, deuterium, hydroxy, amino, mercapto, nitro, halogen (e.g., fluorine, chlorine, bromine, or iodine), cyano, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—), or any combination thereof;
symbol # indicates the point of attachment to U₂; and
n1, n2, n3, n4, m1, m2, and m3 each independently represent an integer of 1, 2, 3, 4, 5, 6, 7, 8, 9, 10, 11, 12, 13, 14, or 15.

In some embodiments, LIN of the compound of Formula (II) represents the structure of the following formula:
#-(C(R^{a1})(R^{a2}))ₙ₁—(O(C(R^{a3})(R^{a4}))ₙ₂)ₘ₁—;
#-(C(R^{a1})(R^{a2}))ₙ₁—(O(C(R^{a3})(R^{a4}))ₙ₂)ₘ₁—(O(C(R^{a5})(R^{a6}))ₙ₃)ₘ₂—;
#-(C(R^{a1})(R^{a2}))ₙ₁—(O(C(R^{a3})(R^{a4}))ₙ₂)ₘ₁—(O(C(R^{a5})(R^{a6}))ₙ₃)ₘ₂—(O(C(R^{a7})(R^{a8}))ₙ₄)ₘ₃—;
#-(C(R^{a1})(R^{a2}))ₙ₁—(N(R_{g})(C(R^{a3})(R^{a4}))ₙ₂)ₘ₁—;
#-(C(R^{a1})(R^{a2}))ₙ₁—(N(R_{g})(C(R^{a3})(R^{a4}))ₙ₂)ₘ₁—(N(R_{g})(C(R^{a5})(R^{a6}))ₙ₃)ₘ₂—;
#-(C(R^{a1})(R^{a2}))ₙ₁—(N(R_{g})(C(R^{a3})(R^{a4}))ₙ₂)ₘ₁—(N(R_{g})(C(R^{a5})(R^{a6}))ₙ₃)ₘ₂—(N(R_{g})(C(R^{a7})(R^{a8}))ₙ₄)ₘ₃—;
#-(C(R^{a1})(R^{a2}))ₙ₁—(C(O)N(R_{g})—(C(R^{a3})(R^{a4}))ₙ₂)ₘ₁—;
#-(C(R^{a1})(R^{a2}))ₙ₁—(C(O)N(R_{g})—(C(R^{a3})(R^{a4}))ₙ₂)ₘ₁—(C(O)N(R_{g})—(C(R^{a5})(R^{a6}))ₙ₃)ₘ₂—;
#-(C(R^{a1})(R^{a2}))ₙ₁—(C(O)N(R_{g})—(C(R^{a3})(R^{a4}))ₙ₂)ₘ₁—(C(O)N(R_{g})—(C(R^{a5})(R^{a6}))ₙ₃)ₘ₂—(C(O)N(R_{g})—
(C(R^{a7})(R^{a8}))ₙ₄)ₘ₃—;
#-(C(R^{a1})(R^{a2}))ₙ₁—(C(O)N(R_{g})—(C(R^{a3})(R^{a4}))ₙ₂)ₘ₁—(O—(C(R^{a5})(R^{a6}))ₙ₃)ₘ₂—;
#-(C(R^{a1})(R^{a2}))ₙ₁—C(O)N(R_{g})—(C(R^{a3})(R^{a4}))ₙ₂—(O(C(R^{a5})(R^{a6}))ₙ₃)ₘ₁—;
#-(C(R^{a1})(R^{a2}))ₙ₁—(N(R_{g})C(O)—(C(R^{a3})(R^{a4}))ₙ₂)ₘ₁—;
#-(C(R^{a1})(R^{a2}))ₙ₁—(N(R_{g})C(O)—(C(R^{a3})(R^{a4}))ₙ₂)ₘ₁—(O—(C(R^{a5})(R^{a6}))ₙ₃)ₘ₂—;
#-(C(R^{a1})(R^{a2}))ₙ₁—(N(R_{g})C(O)—(C(R^{a3})(R^{a4}))ₙ₂)ₘ₁—(O—(C(R^{a5})(R^{a6}))ₙ₃)ₘ₂—(O—(C(R_{g})(R^{a8}))ₙ₄)ₘ₃—;
#-(C(R^{a1})(R^{a2}))ₙ₁—N(R_{g})C(O)—(C(R^{a3})(R^{a4}))ₙ₂—(O(C(R^{a5})(R^{a6}))ₙ₃)ₘ₁—;
#-(C(R^{a1})(R^{a2}))ₙ₁—N(R_{g})C(O)N(R_{g})—(C(R^{a3})(R^{a4}))ₙ₂—;
#-(C(R^{a1})(R^{a2}))ₙ₁—C(O)—(C(R^{a3})(R^{a4}))ₙ₂—;
#-(C(R^{a1})(R^{a2}))ₙ₁-(arylene-(C(R^{a3})(R^{a4}))ₙ₂)ₘ₁—;
#-(C(R^{a1})(R^{a2}))ₙ₁-(arylene-(C(R^{a3})(R^{a4}))ₙ₂)ₘ₁-arylene-(C(R^{a5})(R^{a6}))ₙ₃—;
#-(C(R^{a1})(R^{a2}))ₙ₁-(heterocyclylene-(C(R^{a3})(R^{a4}))ₙ₂)ₘ₁—;
#-(C(R^{a1})(R^{a2}))ₙ₁-(heterocyclylene-(C(R^{a3})(R^{a4}))ₙ₂)ₘ₁—_(heterocyclylene-(C(R^{a5})(R^{a6}))ₙ₃)ₘ₂—;
#-(C(R^{a1})(R^{a2}))ₙ₁-(heteroarylene-(C(R^{a3})(R^{a4}))ₙ₂)ₘ₁—;
#-(C(R^{a1})(R^{a2}))ₙ₁-(heteroarylene-(C(R^{a3})(R^{a4}))ₙ₂)ₘ₁-(heteroarylene-(C(R^{a5})(R^{a6}))ₙ₃)ₘ₂—;
#-(C(R^{a1})(R^{a2}))ₙ₁-(cycloalkylene-(C(R^{a3})(R^{a4}))ₙ₂)ₘ₁—; or
#-(C(R^{a1})(R^{a2}))ₙ₁-(cycloalkylene-(C(R^{a3})(R^{a4}))ₙ₂)ₘ₁-(cycloalkylene-(C(R^{a5})(R^{a6}))ₙ₃)ₘ₂—;
wherein each R_{g} independently represent H or C₁₋₆ alkyl (e.g., C₁₋₄ alkyl, such as methyl, ethyl, propyl, isopropyl, butyl, sec-butyl or tert-butyl);
the cycloalkylene, arylene, heterocyclylene, and heteroarylene are each independently optionally substituted with a substituent selected from the group consisting of deuterium, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), hydroxy, amino, mercapto, halogen (e.g., fluorine, chlorine, bromine, or iodine), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—), cyano, or any combination thereof;
R^{a1}, R^{a2}, R^{a3}, R^{a4}, R^{a5}, R^{a6}, R^{a7} and R^{a8} each independently represent H, deuterium, hydroxy, amino, mercapto, nitro, halogen (e.g., fluorine, chlorine, bromine, or iodine), cyano, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—), or any combination thereof;
symbol # indicates the point of attachment to U₂; and
n1, n2, n3, n4, m1, m2, and m3 each independently represent an integer of 1, 2, 3, 4, 5, 6, 7, 8, 9, 10, 11, 12, 13, 14, or 15.

In some embodiments, LIN of the compound of Formula (II) represents the following groups:
#-CH₂—, #-(CH₂)₂—, #-(CH₂)₃—, #-(CH₂)₄—, #-(CH₂)₅—, #-(CH₂)₆—, #-(CH₂)₇—, #- (CH₂)₈—, #-(CH₂)₉—, #-(CH₂)₁₀—, #-(CH₂)₁₁—, #-(CH₂)₁₂—, #-(CH₂)₁₃—, #-(CH₂)₁₄—, #-(CH₂)₁₅—, #-(CH₂)₁₆—, #-(CH₂)₁₇—, #-(CH₂)₁₈—, #-(CH₂)₁₉—, #-(CH₂)₂₀—, #-(CH₂)₂₁—, #-(CH₂)₂₂—, #-(CH₂)₂₅—, or #-(CH₂)₃₀—; #-CH₂—O—CH₂—, #-CH₂—O—(CH₂)₂—, #-(CH₂)₁—O—(CH₂)₃—, #-(CH₂)₁—O—(CH₂)₄—, #-(CH₂)₁—O—(CH₂)₅—, #-(CH₂)₁—O—(CH₂)₆—, #-(CH₂)₁—O—(CH₂)₇—, #-(CH₂)₁—O—(CH₂)₈—, #-(CH₂)₁—O—(CH₂)₉—, #-(CH₂)₁—O—(CH₂)₁₀—, #-(CH₂)₂—O—(CH₂)₁—, #-(CH₂)₂—O—(CH₂)₂—, #-(CH₂)₂—O—(CH₂)₃—, #-(CH₂)₂—O—(CH₂)₄—, #-(CH₂)₂—O—(CH₂)₅—, #-(CH₂)₂—O—(CH₂)₆—, #-(CH₂)₂—O—(CH₂)₇—, #-(CH₂)₂—O—(CH₂)₈—, #-(CH₂)₂—O—(CH₂)₉—, #-(CH₂)₂—O—(CH₂)₁₀—, #-(CH₂)₂—O—(CH₂)₁₁—, #-(CH₂)₂—O—(CH₂)₁₂—, #-(CH₂)₃—O—(CH₂)₁—, #-(CH₂)₃—O—(CH₂)₂—, #-(CH₂)₃—O—(CH₂)₃—, #-(CH₂)₃—O—(CH₂)₄—, #-(CH₂)₃—O—(CH₂)₅—, #-(CH₂)₃—O—(CH₂)₆—, #-(CH₂)₃—O—(CH₂)₇—, #-(CH₂)₄—O—(CH₂)₁—, #-(CH₂)₄—O—(CH₂)₂—, #-(CH₂)₄—O—(CH₂)₃—, #-(CH₂)₄—O—(CH₂)₄—, #-(CH₂)₄—O—(CH₂)₅—, #-(CH₂)₄—O—(CH₂)₆—, #-(CH₂)₅—O—(CH₂)₁—, #-(CH₂)₅—O—(CH₂)₂—, #-(CH₂)₅—O—(CH₂)₃—, #-(CH₂)₅—O—(CH₂)₄—, #-(CH₂)₅—O—(CH₂)₅—, #-(CH₂)₆—O—(CH₂)₁—, #-(CH₂)₆—O—(CH₂)₂—, #-(CH₂)₆—O—(CH₂)₃—, #-(CH₂)₆—O—(CH₂)₄—, #-(CH₂)₇—O—(CH₂)₁—, #-(CH₂)₇—O—(CH₂)₂—, #-(CH₂)₇—O—(CH₂)₃—, #-(CH₂)₈—O—(CH₂)₁—, #-(CH₂)₈—O—(CH₂)₂—, #-CH(CH₃)—O—(CH₂)₁—, #-CH(CH₃)—O—(CH₂)₂—, #-CH(CH₃)—O—(CH₂)₃—, #-CH(CH₃)—O—(CH₂)₄—, #-CH(CH₃)—O—(CH₂)₅—, #-CH(CH₃)—O—(CH₂)₆—, #-CH(CH₃)—O—(CH₂)₇—, #-CH(CH₃)—O—(CH₂)₈—, #-CH(CH₃)—O—(CH₂)₉—, #-CH(CH₃)—O—(CH₂)₁₀—, #-CH₂—(O(CH₂)₂)₂—, #-CH₂—(O(CH₂)₂)₃—, #-CH₂—(O(CH₂)₂)₄—, #-CH₂—(O(CH₂)₂)₅—, #-CH₂—(O(CH₂)₂)₆—, #-CH₂—(O(CH₂)₂)₇—, #-CH₂—(O(CH₂)₂)₈—, #-CH₂—(O(CH₂)₂)₉—, #-CH₂—(O(CH₂)₂)₁₀—, #-CH₂—(O(CH₂)₂)₁—OCH₂—, #-CH₂—(O(CH₂)₂)₂—OCH₂—, #-CH₂—(O(CH₂)₂)₃—OCH₂—, #-CH₂—(O(CH₂)₂)₄—OCH₂—, #-CH₂—(O(CH₂)₂)₅—OCH₂—, #-CH₂—(O(CH₂)₂)₆—OCH₂—, #-CH₂—(O(CH₂)₂)₇—OCH₂—, #-CH₂—(O(CH₂)₂)₈—OCH₂—, #-CH₂—(O(CH₂)₂)₉—OCH₂—, #-CH₂—(O(CH₂)₂)₁₀—OCH₂—, #-(CH₂)₂—(O(CH₂)₂)₂—, #-(CH₂)₂—(O(CH₂)₂)₃—, #-(CH₂)₂—(O(CH₂)₂)₄—, #-(CH₂)₂—(O(CH₂)₂)₅—, #-(CH₂)₂—(O(CH₂)₂)₆—, #-(CH₂)₂—(O(CH₂)₂)₇—, #-(CH₂)₂—(O(CH₂)₂)₈—, #-(CH₂)₂—(O(CH₂)₂)₉—, #-(CH₂)₂—(O(CH₂)₂)₁₀—, #-(CH₂)₃—(O(CH₂)₂)₂—, #-(CH₂)₃—(O(CH₂)₂)₃—, #-(CH₂)₃—(O(CH₂)₂)₄—, #-(CH₂)₃—(O(CH₂)₂)₅—, #-(CH₂)₃—(O(CH₂)₂)₆—, #-(CH₂)₃—(O(CH₂)₂)₇—, #-(CH₂)₃—(O(CH₂)₂)₈—, #-(CH₂)₃—(O(CH₂)₂)₉—, #-(CH₂)₃—(O(CH₂)₂)₁₀—, #-(CH₂)₄—(O(CH₂)₂)₂—, #-(CH₂)₄—(O(CH₂)₂)₃—, #-(CH₂)₄—(O(CH₂)₂)₄—, #-(CH₂)₄—(O(CH₂)₂)₅—, #-(CH₂)₄—(O(CH₂)₂)₆—, #-(CH₂)₄—(O(CH₂)₂)₇—, #-(CH₂)₄—(O(CH₂)₂)₈—, #-(CH₂)₄—(O(CH₂)₂)₉—, #-(CH₂)₄—(O(CH₂)₂)₁₀—, #-CH₂—(O(CH₂)₃)₂—, #-CH₂—(O(CH₂)₃)₃—, #-CH₂—(O(CH₂)₃)₄—, #-CH₂—(O(CH₂)₃)₅—, #-CH₂—(O(CH₂)₃)₆—, #-CH₂—(O(CH₂)₃)₇—, #-CH₂—(O(CH₂)₃)₈—, #-CH₂—(O(CH₂)₃)₉—, #- CH₂—(O(CH₂)₃)₁₀—, #-(CH₂)₂—(O(CH₂)₃)₂—, #-(CH₂)₂—(O(CH₂)₃)₃—, #-(CH₂)₂—(O(CH₂)₃)₄—, #-(CH₂)₂—(O(CH₂)₃)₅—, #-(CH₂)₂—(O(CH₂)₃)₆—, #-(CH₂)₂—(O(CH₂)₃)₇—, #-(CH₂)₂—(O(CH₂)₃)₈—, #-(CH₂)₂—(O(CH₂)₃)₉—, #-(CH₂)₂—(O(CH₂)₃)₁₀—, #-(CH₂)₃—(O(CH₂)₃)₂—, #-(CH₂)₃—(O(CH₂)₃)₃—, #-(CH₂)₃—(O(CH₂)₃)₄—, #-(CH₂)₃—(O(CH₂)₃)₅—, #-(CH₂)₃—(O(CH₂)₃)₆—, #-(CH₂)₃—(O(CH₂)₃)₇—, #-(CH₂)₃—(O(CH₂)₃)₈—, #-(CH₂)₃—(O(CH₂)₃)₉—, #-(CH₂)₃—(O(CH₂)₃)₁₀—, #-CH₂—O—(CH₂)₂—O—(CH₂)₃—, #-CH₂—(O(CH₂)₂)₂—(O(CH₂)₃)₂—, #-CH₂—(O(CH₂)₂)₃—(O(CH₂)₃)₃—, #-CH₂—(O(CH₂)₂)₄—(O(CH₂)₃)₄—, #-CH₂—(O(CH₂)₂)₅—(O(CH₂)₃)₅—, #-CH₂—(O(CH₂)₂)₆—(O(CH₂)₃)₆—, #-(CH₂)₂—O—(CH₂)₂—O—(CH₂)₃—, #-(CH₂)₂—(O(CH₂)₂)₂—(O(CH₂)₃)₂—, #-(CH₂)₂—(O(CH₂)₂)₃—(O(CH₂)₃)₃—, #-(CH₂)₂—(O(CH₂)₂)₄—(O(CH₂)₃)₄—, #-(CH₂)₂—(O(CH₂)₂)₅—(O(CH₂)₃)₅—, #-(CH₂)₂—(O(CH₂)₂)₆—(O(CH₂)₃)₆—, #-(CH₂)₃—O—(CH₂)₂—O—(CH₂)₃—, #-(CH₂)₃—(O(CH₂)₂)₂—(O(CH₂)₃)₂—, #-(CH₂)₃—(O(CH₂)₂)₃—(O(CH₂)₃)₃—, #-(CH₂)₃—(O(CH₂)₂)₄—(O(CH₂)₃)₄—, #-(CH₂)₃—(O(CH₂)₂)₅—(O(CH₂)₃)₅—, #-(CH₂)₃—(O(CH₂)₂)₆—(O(CH₂)₃)₆—, #-CH₂—O—(CH₂)₃—O—(CH₂)₂—, #-CH₂—(O(CH₂)₃)₂—(O(CH₂)₂)₂—, #-CH₂—(O(CH₂)₃)₃—(O(CH₂)₂)₃—, #-CH₂—(O(CH₂)₃)₄—(O(CH₂)₂)₄—, #-CH₂—(O(CH₂)₃)₅—(O(CH₂)₂)₅—, #-CH₂—(O(CH₂)₃)₆—(O(CH₂)₂)₆—, #-(CH₂)₂—O—(CH₂)₃—O—(CH₂)₂—, #-(CH₂)₂—(O(CH₂)₃)₂—(O(CH₂)₂)₂—, #-(CH₂)₂—(O(CH₂)₃)₃—(O(CH₂)₂)₃—, #-(CH₂)₂—(O(CH₂)₃)₄—(O(CH₂)₂)₄—, #-(CH₂)₂—(O(CH₂)₃)₅—(O(CH₂)₂)₅—, #-(CH₂)₂—(O(CH₂)₃)₆—(O(CH₂)₂)₆—, #-(CH₂)₃—O—(CH₂)₃—O—(CH₂)₂—, #-(CH₂)₃—(O(CH₂)₃)₂—(O(CH₂)₂)₂—, #-(CH₂)₃—(O(CH₂)₃)₃—(O(CH₂)₂)₃—, #-(CH₂)₃—(O(CH₂)₃)₄—(O(CH₂)₂)₄—, #-(CH₂)₃—(O(CH₂)₃)₅—(O(CH₂)₂)₅—, #-(CH₂)₃—(O(CH₂)₃)₆—(O(CH₂)₂)₆—, #-CH₂—O—(CH₂)₂O—CH₂—, #-(CH₂)₂—O—(CH₂)₂O—CH₂—, #-(CH₂)₂—(O(CH₂)₂)₂—O—(CH₂)₃—, #-(CH₂)₂—(O(CH₂)₂)₃—O—(CH₂)₃—, #-(CH₂)₂—(O(CH₂)₂)₄—O—(CH₂)₃—, #-(CH₂)₅—(O(CH₂)₂)₂—O—(CH₂)₅—, #-(CH₂)₅—(O(CH₂)₂)₂—O—(CH₂)₆—, #-(CH₂)₁—N(R_{g})—(CH₂)₁—, #-(CH₂)₁—N(R_{g})—(CH₂)₂—, #-(CH₂)₁—N(R_{g})—(CH₂)₃—, #-(CH₂)₁—N(R_{g})—(CH₂)₄—, #-(CH₂)₁—N(R_{g})—(CH₂)₅—, #-(CH₂)₁—N(R_{g})—(CH₂)₆—, #-(CH₂)₁—N(R_{g})—(CH₂)₇—, #-(CH₂)₁—N(R_{g})—(CH₂)₈—, #-(CH₂)₁—N(R_{g})—(CH₂)₉—, #-(CH₂)₁—N(R_{g})—(CH₂)₁₀—, #-(CH₂)₂—N(R_{g})—(CH₂)₁—, #-(CH₂)₂—N(R_{g})—(CH₂)₂—, #-(CH₂)₂—N(R_{g})—(CH₂)₃—, #-(CH₂)₂—N(R_{g})—(CH₂)₄—, #-(CH₂)₂—N(R_{g})—(CH₂)₅—, #-(CH₂)₂—N(R_{g})—(CH₂)₆—, #-(CH₂)₂—N(R_{g})—(CH₂)₇—, #-(CH₂)₂—N(R_{g})—(CH₂)₈—, #-(CH₂)₂—N(R_{g})—(CH₂)₉—, #-(CH₂)₂—N(R_{g})—(CH₂)₁₀—, #-(CH₂)₂—N(R_{g})—(CH₂)₁₁—, #-(CH₂)₂—N(R_{g})—(CH₂)₁₂—, #-(CH₂)₃—N(R_{g})—(CH₂)₁—, #-(CH₂)₃—N(R_{g})—(CH₂)₂—, #-(CH₂)₃—N(R_{g})—(CH₂)₃—, #-(CH₂)₄—N(R_{g})—(CH₂)₁—, #-(CH₂)₄—N(R_{g})—(CH₂)₂—, #-(CH₂)₄—N(R_{g})—(CH₂)₃—, #-(CH₂)₄—N(R_{g})—(CH₂)₄—, #-(CH₂)₅—N(R_{g})—(CH₂)₁—, #-(CH₂)₅—N(R_{g})—(CH₂)₂—, #-(CH₂)₅—N(R_{g})—(CH₂)₃—, #-(CH₂)₅—N(R_{g})—(CH₂)₄—, #-(CH₂)₅—N(R_{g})—(CH₂)₅—, #-(CH₂)₆—N(R_{g})—(CH₂)₁—, #-(CH₂)₆—N(R_{g})—(CH₂)₂—, #-(CH₂)₆—N(R_{g})—(CH₂)₃—, #-(CH₂)₇—N(R_{g})—(CH₂)₁—, #-(CH₂)₇—N(R_{g})—(CH₂)₂—, #-(CH₂)₇—N(R_{g})—(CH₂)₃—, #-(CH₂)₈—N(R_{g})—(CH₂)₁—, #-(CH₂)₈—N(R_{g})—(CH₂)₂—, #-(CH₂)₈—N(R_{g})—(CH₂)₃—, #-CH(CH₃)—N(R_{g})—(CH₂)₁—, #-CH(CH₃)—N(R_{g})—(CH₂)₂—, #-CH(CH₃)—N(R_{g})—(CH₂)₃—, #-CH(CH₃)—N(R_{g})—(CH₂)₄—, #-CH(CH₃)—N(R_{g})—(CH₂)₅—, #-CH(CH₃)—N(R_{g})—(CH₂)₆—, #-CH(CH₃)—N(R_{g})—(CH₂)₇—, #-CH(CH₃)—N(R_{g})—(CH₂)₈—, #-CH(CH₃)—N(R_{g})—(CH₂)₉—, #-CH(CH₃)—N(R_{g})—(CH₂)₁₀—, #-CH₂C(O)NHCH₂—, #-(CH₂)₂C(O)NH(CH₂)₂—, #-(CH₂)₂C(O)NH(CH₂)₃—, #-(CH₂)₂C(O)NH(CH₂)₄—, #-(CH₂)₂C(O)NH(CH₂)₅—, #-(CH₂)₃C(O)NH(CH₂)₃—, #-(CH₂)₃C(O)NH(CH₂)₄—, #-(CH₂)₄C(O)NH(CH₂)₄—, #-(CH₂)₅C(O)NH(CH₂)₅—, #-(CH₂)₆C(O)NH(CH₂)₇—, #-(CH₂)₆C(O)NH(CH₂)₆—, #-(CH₂)₇C(O)NH(CH₂)₇—, #-(CH₂)₈C(O)NH(CH₂)₈, U—(CH₂)₉C(O)NH(CH₂)₉—, #-(CH₂)₁₀C(O)NH(CH₂)₁₀—, #-(CH₂)₂C(O)NH(CH₂)₂—O—(CH₂)₂—, #-CH₂NHC(O)CH₂—, #-(CH₂)₂NHC(O)(CH₂)₂—, #-(CH₂)₂NHC(O)(CH₂)₃—, #-(CH₂)₂NHC(O)(CH₂)₄—, #-(CH₂)₂NHC(O)(CH₂)₅—, #-(CH₂)₃NHC(O)(CH₂)₃—, #-(CH₂)₃NHC(O)(CH₂)₄—, #-(CH₂)₄NHC(O)(CH₂)₄—, #-(CH₂)₅NHC(O)(CH₂)₅—, #-(CH₂)₆NHC(O)(CH₂)₇—, #-(CH₂)₆NHC(O)(CH₂)₆—, #-(CH₂)₇NHC(O)(CH₂)₇—, #-(CH₂)₈NHC(O)(CH₂)₈, #-(CH₂)₉NHC(O)(CH₂)₉—, #-(CH₂)₁₀NHC(O)(CH₂)₁₀—, #-(CH₂)₄NHC(O)(CH₂)₈—, #-(CH₂)₂NHC(O)(CH₂)₂—O—(CH₂)₂—, #-(CH₂)₄NHC(O)CH₂—, #-CH₂-piperidinylene-CH₂—, #-CH₂-piperidinylene-(CH₂)₂—, #-CH₂-piperidinylene-(CH₂)₃—, #-CH₂-piperidinylene-(CH₂)₄—, #-CH₂-piperidinylene-(CH₂)₅—, #-CH₂-piperidinylene-(CH₂)₆—, #-CH₂-piperidinylene-(CH₂)₇—, #-CH₂-piperidinylene-(CH₂)₈—, #-(CH₂)₂-piperidinylene-(CH₂)₁—, #-(CH₂)₂-piperidinylene-(CH₂)₂—, #-(CH₂)₂-piperidinylene-(CH₂)₃—, #-(CH₂)₂-piperidinylene-(CH₂)₄—, #-(CH₂)₂-piperidinylene-(CH₂)₅—, #-(CH₂)₂-piperidinylene-(CH₂)₆—, #-(CH₂)₂-piperidinylene-(CH₂)₇—, #-(CH₂)₂-piperidinylene-(CH₂)₈—, #-(CH₂)₃-piperidinylene-CH₂—, #-(CH₂)₃-piperidinylene-(CH₂)₂—, #-(CH₂)₃-piperidinylene-(CH₂)₃—, #-(CH₂)₃-piperidinylene-(CH₂)₄—, #-(CH₂)₃-piperidinylene-(CH₂)₅—, #-(CH₂)₃-piperidinylene-(CH₂)₆—, #-(CH₂)₃-piperidinylene-(CH₂)₇—, #-(CH₂)₃-piperidinylene-(CH₂)₈—, #-(CH₂)₄-piperidinylene-CH₂—, #-(CH₂)₄-piperidinylene-(CH₂)₂—, #-(CH₂)₄-piperidinylene-(CH₂)₃—, #-(CH₂)₄-piperidinylene-(CH₂)₄—, #-(CH₂)₄-piperidinylene-(CH₂)₅—, #-(CH₂)₄-piperidinylene-(CH₂)₆—, #-(CH₂)₄-piperidinylene-(CH₂)₇—, #-(CH₂)₄-piperidinylene-(CH₂)₈—, #-(CH₂)₅-piperidinylene-(CH₂)₁—, #-(CH₂)₅-piperidinylene-(CH₂)₂—, #-(CH₂)₅-piperidinylene-(CH₂)₃—, #-(CH₂)₅-piperidinylene-(CH₂)₄—, #-(CH₂)₅-piperidinylene-(CH₂)₅—, #-(CH₂)₅-piperidinylene-(CH₂)₆—, #-(CH₂)₅-piperidinylene-(CH₂)₇—, #-(CH₂)₅-piperidinylene-(CH₂)₈—, #-(CH₂)₆-piperidinylene-(CH₂)₁—, #-(CH₂)₆-piperidinylene-(CH₂)₂—, #-(CH₂)₆-piperidinylene-(CH₂)₃—, #-(CH₂)₆-piperidinylene-(CH₂)₄—, #-(CH₂)₆-piperidinylene-(CH₂)₅—, #-(CH₂)₆-piperidinylene-(CH₂)₆—, #-(CH₂)₆-piperidinylene-(CH₂)₇—, #-(CH₂)₆-piperidinylene-(CH₂)₈—, #-(CH₂)₇-piperidinylene-(CH₂)₁—, #-(CH₂)₇-piperidinylene-(CH₂)₂—, #-(CH₂)₇-piperidinylene-(CH₂)₃—, #-(CH₂)₇-piperidinylene-(CH₂)₄—, #-(CH₂)₇-piperidinylene-(CH₂)₈—, #-(CH₂)₈-piperidinylene-CH₂—, #-(CH₂)₈-piperidinylene-(CH₂)₂—, #-(CH₂)₈-piperidinylene-(CH₂)₃—, #-(CH₂)₈-piperidinylene-(CH₂)₄—, #-(CH₂)₈-piperidinylene-(CH₂)₅—, #-(CH₂)₈-piperidinylene-(CH₂)₆—, #-(CH₂)₈-piperidinylene-(CH₂)₇—, #-(CH₂)₈-piperidinylene-(CH₂)₈—, #-CH₂—N(R_{g})—CH₂-piperidinylene-CH₂—, #-CH₂—N(R_{g})—CH₂-piperidinylene-(CH₂)₂—, #-CH₂—N(R_{g})—CH₂-piperidinylene-(CH₂)₃—, #-CH₂—N(R_{g})—CH₂-piperidinylene-(CH₂)₄—, #-CH₂—N(R_{g})—CH₂-piperidinylene-(CH₂)₅—, #-CH₂—N(R_{g})—CH₂-piperidinylene-(CH₂)₆—, #-CH₂—N(R_{g})—CH₂-piperidinylene-(CH₂)₇—, #-CH₂—N(R_{g})—CH₂-piperidinylene-(CH₂)₈—, #-CH₂—N(R_{g})—(CH₂)₂-piperidinylene-CH₂—, #-CH₂—N(R_{g})—(CH₂)₂-piperidinylene-(CH₂)₂—, #-CH₂—N(R_{g})—(CH₂)₂-piperidinylene-(CH₂)₃—, #-CH₂—N(R_{g})—(CH₂)₂-piperidinylene-(CH₂)₄—, #-CH₂—N(R_{g})—(CH₂)₂-piperidinylene-(CH₂)₅—, #-CH₂—N(R_{g})—(CH₂)₂-piperidinylene-(CH₂)₆—, #-CH₂—N(R_{g})—(CH₂)₂-piperidinylene-(CH₂)₇—, #-CH₂—N(R_{g})—(CH₂)₂-piperidinylene-(CH₂)₈—, #-(CH₂)₂—N(R_{g})—CH₂-piperidinylene-CH₂—, #-(CH₂)₂—N(R_{g})—CH₂-piperidinylene-(CH₂)₂—, #-(CH₂)₂—N(R_{g})—CH₂-piperidinylene-(CH₂)₃—, #-(CH₂)₂—N(R_{g})—CH₂-piperidinylene-(CH₂)₄—, #-(CH₂)₂—N(R_{g})—CH₂-piperidinylene-(CH₂)₅—, #-(CH₂)₂—N(R_{g})—CH₂-piperidinylene-(CH₂)₆—, #-(CH₂)₂—N(R_{g})—CH₂-piperidinylene-(CH₂)₇—, #-(CH₂)₂—N(R_{g})—CH₂-piperidinylene-(CH₂)₈—, #-(CH₂)₃—N(R_{g})—CH₂-piperidinylene-CH₂—, #-(CH₂)₃—N(R_{g})—CH₂-piperidinylene-(CH₂)₂—, #-(CH₂)₃—N(R_{g})—CH₂-piperidinylene-(CH₂)₃—, #-(CH₂)₃—N(R_{g})—CH₂-piperidinylene-(CH₂)₈—, #-(CH₂)₄—N(R_{g})—CH₂-piperidinylene-CH₂—, #-(CH₂)₄—N(R_{g})—CH₂-piperidinylene-(CH₂)₂—, #-(CH₂)₄—N(R_{g})—CH₂-piperidinylene-(CH₂)₃—, #-(CH₂)₄—N(R_{g})—CH₂-piperidinylene-(CH₂)₈—, #-(CH₂)₅—N(R_{g})—CH₂-piperidinylene-(CH₂)₃—, #-(CH₂)₅—N(R_{g})—CH₂-piperidinylene-(CH₂)₈—, #-(CH₂)₆—N(R_{g})—CH₂-piperidinylene-(CH₂)₃—, #-(CH₂)₆—N(R_{g})—CH₂-piperidinylene-(CH₂)₈—, #-(CH₂)₇—N(R_{g})—CH₂-piperidinylene-(CH₂)₃—, #-(CH₂)₇—N(R_{g})—CH₂-piperidinylene-(CH₂)₈—, #-(CH₂)₈—N(R_{g})—CH₂-piperidinylene-CH₂—, #-(CH₂)₈—N(R_{g})—CH₂-piperidinylene-(CH₂)₂—, #-(CH₂)₈—N(R_{g})—CH₂-piperidinylene-(CH₂)₃—, #-(CH₂)₈—N(R_{g})—CH₂-piperidinylene-(CH₂)₄—, #-(CH₂)₈—N(R_{g})—CH₂-piperidinylene-(CH₂)₅—, #-(CH₂)₈—N(R_{g})—CH₂-piperidinylene-(CH₂)₆—, #-(CH₂)₈—N(R_{g})—CH₂-piperidinylene-(CH₂)₇—, #-(CH₂)₈—N(R_{g})—CH₂-piperidinylene-(CH₂)₈—, #-CH₂-piperazinylene-CH₂—, #-CH₂-piperazinylene-(CH₂)₂—, #-CH₂-piperazinylene-(CH₂)₃—, #-CH₂-piperazinylene-(CH₂)₄—, #-CH₂-piperazinylene-(CH₂)₅—, #-CH₂-piperazinylene-(CH₂)₆—, #-CH₂-piperazinylene-(CH₂)₇—, #-CH₂-piperazinylene-(CH₂)₈—, #-(CH₂)₂-piperazinylene-(CH₂)₁—, #-(CH₂)₂-piperazinylene-(CH₂)₂—, #-(CH₂)₂-piperazinylene-(CH₂)₃—, #-(CH₂)₂-piperazinylene-(CH₂)₄—, #-(CH₂)₂-piperazinylene-(CH₂)₅—, #-(CH₂)₂-piperazinylene-(CH₂)₆—, #-(CH₂)₂-piperazinylene-(CH₂)₇—, #-(CH₂)₂-piperazinylene-(CH₂)₈—, #-(CH₂)₃-piperazinylene-CH₂—, #-(CH₂)₃-piperazinylene-(CH₂)₂—, #-(CH₂)₃-piperazinylene-(CH₂)₃—, #-(CH₂)₃-piperazinylene-(CH₂)₄—, #-(CH₂)₃-piperazinylene-(CH₂)₅—, #-(CH₂)₃-piperazinylene-(CH₂)₆—, #-(CH₂)₃-piperazinylene-(CH₂)₇—, #-(CH₂)₃-piperazinylene-(CH₂)₈—, #-(CH₂)₄-piperazinylene-CH₂—, #-(CH₂)₄-piperazinylene-(CH₂)₂—, #-(CH₂)₄-piperazinylene-(CH₂)₃—, #-(CH₂)₄-piperazinylene-(CH₂)₄—, #-(CH₂)₄-piperazinylene-(CH₂)₅—, #-(CH₂)₄-piperazinylene-(CH₂)₆—, #-(CH₂)₄-piperazinylene-(CH₂)₇—, #-(CH₂)₄-piperazinylene-(CH₂)₈—, #-(CH₂)₅-piperazinylene-(CH₂)₁—, #-(CH₂)₅-piperazinylene-(CH₂)₂—, #-(CH₂)₅-piperazinylene-(CH₂)₃—, #-(CH₂)₅-piperazinylene-(CH₂)₄—, #-(CH₂)₅-piperazinylene-(CH₂)₅—, #-(CH₂)₅-piperazinylene-(CH₂)₆—, #-(CH₂)₅-piperazinylene-(CH₂)₇—, #-(CH₂)₅-piperazinylene-(CH₂)₈—, #-(CH₂)₆-piperazinylene-(CH₂)₁—, #-(CH₂)₆-piperazinylene-(CH₂)₂—, #-(CH₂)₆-piperazinylene-(CH₂)₃—, #-(CH₂)₆-piperazinylene-(CH₂)₄—, #-(CH₂)₆-piperazinylene-(CH₂)₅—, #-(CH₂)₆-piperazinylene-(CH₂)₆—, #-(CH₂)₆-piperazinylene-(CH₂)₇—, #-(CH₂)₆-piperazinylene-(CH₂)₈—, #-(CH₂)₇-piperazinylene-(CH₂)₁—, #-(CH₂)₇-piperazinylene-(CH₂)₂—, #-(CH₂)₇-piperazinylene-(CH₂)₃—, #-(CH₂)₇-piperazinylene-(CH₂)₄—, #-(CH₂)₇-piperazinylene-(CH₂)₈—, #-(CH₂)₈-piperazinylene-CH₂—, #-(CH₂)₈-piperazinylene-(CH₂)₂—, #-(CH₂)₈-piperazinylene-(CH₂)₃—, #-(CH₂)₈-piperazinylene-(CH₂)₄—, #-(CH₂)₈-piperazinylene-(CH₂)₅—, #-(CH₂)₈-piperazinylene-(CH₂)₆—, #-(CH₂)₈-piperazinylene-(CH₂)₇—, or #-(CH₂)₈-piperazinylene-(CH₂)₈—;
wherein the piperidinylene and the piperazinylene are each independently optionally substituted with a substituent selected from the group consisting of deuterium, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), hydroxy, amino, mercapto, halogen (e.g., fluorine, chlorine, bromine, or iodine), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—), cyano, or any combination thereof;
each R_{g} independently represent H or C₁₋₆ alkyl (e.g., C₁₋₄ alkyl, such as methyl, ethyl, propyl, isopropyl, butyl, sec-butyl or tert-butyl);
symbol # indicates the point of attachment to U₂; and
n1, n2, n3, n4, m1, m2, and m3 each independently represent an integer of 1, 2, 3, 4, 5, 6, 7, 8, 9, 10, 11, 12, 13, 14, or 15.

In some embodiments, Rₐ of the compound of Formula (II) represents the following groups:

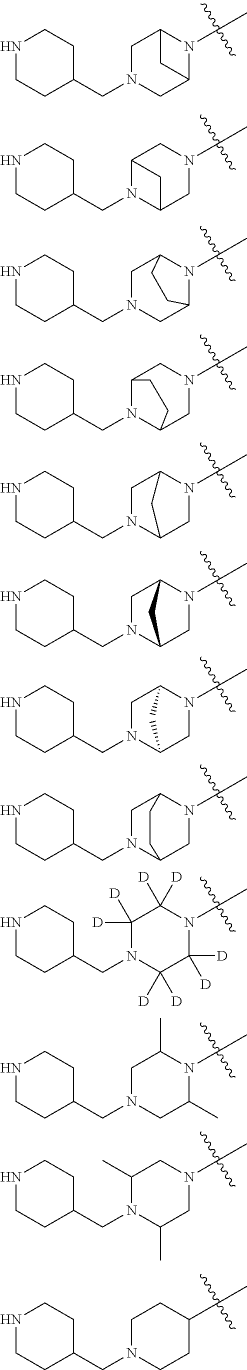

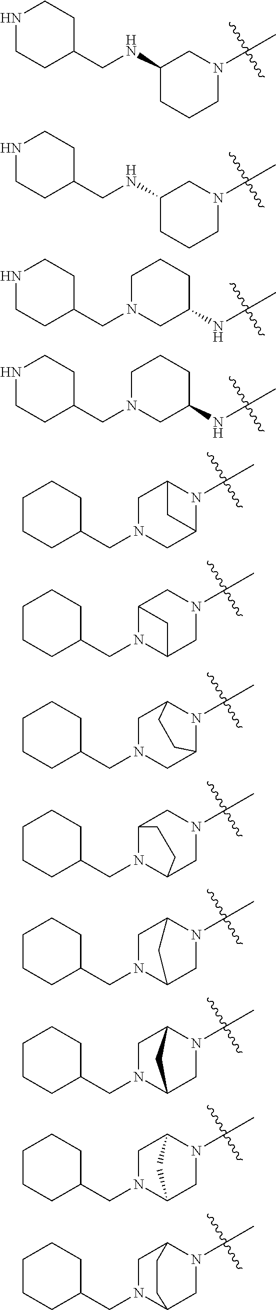

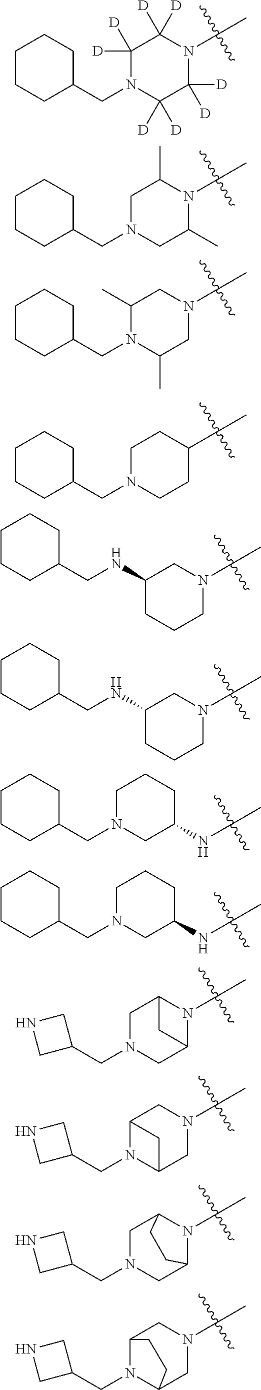

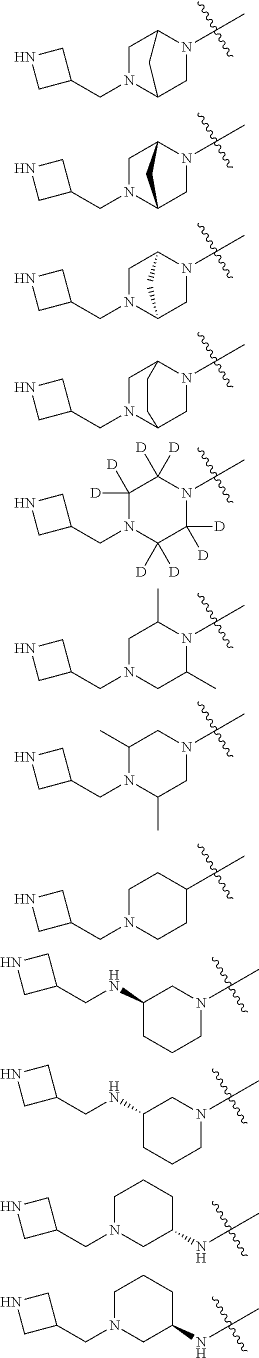

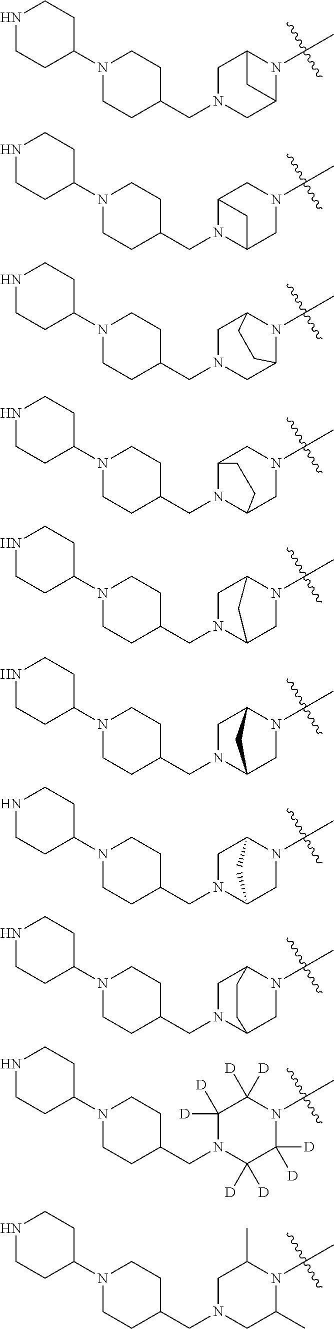

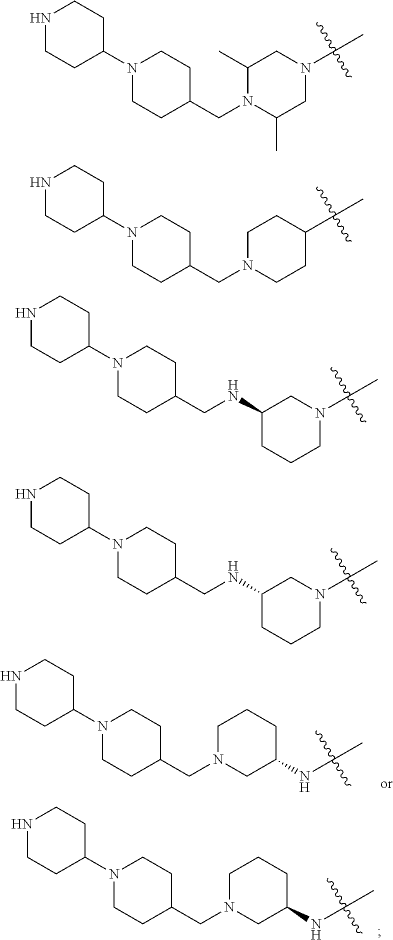 [SMILES: *N1CC(C)N(CC2CCN(C3CCNCC3)CC2)C(C)C1 ; *C1CCN(CC2CCN(C3CCNCC3)CC2)CC1 ; *N1CCC[C@@H](NCC2CCN(C3CCNCC3)CC2)C1 ; *N1CCC[C@H](NCC2CCN(C3CCNCC3)CC2)C1 ; *N[C@H]1CCCN(CC2CCN(C3CCNCC3)CC2)C1 ; *N[C@@H]1CCCN(CC2CCN(C3CCNCC3)CC2)C1]

wherein the groups are optionally partially or fully deuterated.

In some embodiments, the compound of Formula (II) is also of Formula (II-1) or Formula (II-2):

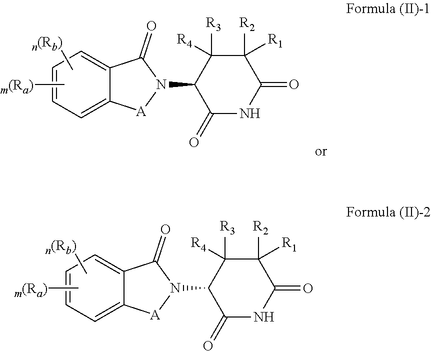

wherein A, R₁, R₂, R₃, R₄, (R_{b})ₙ and (Rₐ)ₘ are as defined in the compound of Formula (II) above and embodiments thereof.

In some embodiments, the compound of Formula (II) is also of Formula (II-3) or Formula (II-4):

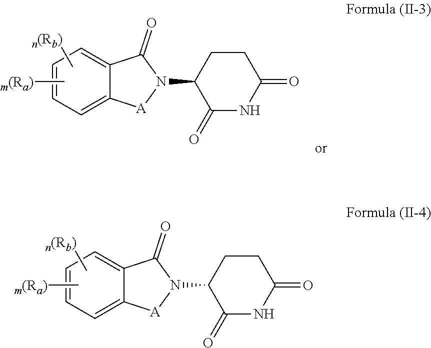

wherein A, (R_{b})ₙ and (Rₐ)ₘ are as defined in the compound of Formula (II) above and embodiments thereof.

In some embodiments, the compound of Formula (II) is also of Formula (II-5), Formula (II-6), Formula (II-7) or Formula (II-8):

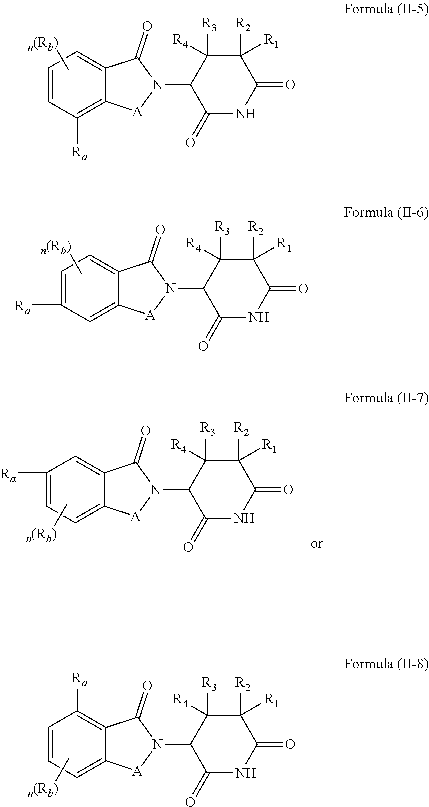

wherein A, R₁, R₂, R₃, R₄, (R_{b})ₙ and Rₐ are as defined in the compound of Formula (II) above and embodiments thereof.

In some embodiments, in the compounds of Formula (II-5), Formula (II-6), Formula (II-7) or Formula (II-8),
A represents CO, CH₂ or CD₂;
R₁, R₂, R₃ and R₄ are the same or different and each independently represent hydrogen, deuterium, halogen (e.g., fluorine, chlorine, bromine, or iodine), optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—);
(R_{b})ₙ indicates that benzene ring is substituted with n R_{b} groups, and each R_{b} are the same or different and each independently represent hydrogen, deuterium, halogen (e.g., fluorine, chlorine, bromine, or iodine), hydroxy, mercapto, nitro, amino, cyano, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₂₋₆ alkenyl (e.g., vinyl, propenyl, or butenyl), or C₂₋₆ alkynyl (e.g., ethynyl, propynyl, or butynyl);
wherein n represents an integer of 0, 1, 2, or 3;
Rₐ represents the structure of the following formula:

W-LIN—U₂—R_{c}—U₁—,
wherein U₁ and U₂ are the same or different and each independently represent a bond, N(R_{d}) or O, wherein R_{d} represents H or optionally deuterated C₁₋₆ alkyl (e.g., optionally deuterated C₁₋₄ alkyl, such as methyl, CD₃, ethyl, propyl, isopropyl, butyl, sec-butyl or tert-butyl);
R_{c} represents optionally substituted nitrogen-containing monocyclic heterocyclylene or optionally substituted nitrogen-containing bridged heterocyclylene;
W represents hydrogen, optionally substituted nitrogen-containing monocyclic heterocyclyl, optionally substituted nitrogen-containing bridged heterocyclyl, optionally substituted cycloalkyl, optionally substituted heteroaryl, optionally substituted aryl, C₂₋₆ alkenyl (e.g., vinyl, propenyl, or butenyl), C₂₋₆ alkynyl (e.g., ethynyl, propynyl, or butynyl), a leaving group, CHO, COOH, NH₂, hydroxy or mercapto; and
wherein in case that the compound is partially or fully deuterated:
   when W represents optionally substituted nitrogen-containing monocyclic heterocyclyl, optionally substituted nitrogen-containing bridged heterocyclyl, optionally substituted cycloalkyl, optionally substituted heteroaryl or optionally substituted aryl, LIN represents a bond, optionally substituted methylene, or optionally substituted linear or branched C₂₋₃₀ alkylene, wherein one or more (e.g., 1-10, 1-8 or 1-6, such as 1, 2, 3, 4, 5 or 6) groups Rₑ and/or one or more (e.g., 1-10, 1-8 or 1-6, such as 1, 2, 3, 4, 5 or 6) groups R_{f} and/or any combination of one or more (e.g., 1-10, 1-8 or 1-6, such as 1, 2, 3, 4, 5 or 6) groups Rₑ and R_{f} are optionally inserted into the backbone carbon chain of the linear or branched C₂₋₃₀ alkylene group, wherein carbon-carbon bond between one or more (e.g., 1-10, 1-8 or 1-6, such as 1, 2, 3, 4, 5 or 6) pairs of adjacent carbon atoms in the backbone carbon chain is interrupted by the group Rₑ, R_{f}, or a combination of Rₑ and R_{f}; wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl (e.g., C₁₋₄ alkyl, such as methyl, ethyl, propyl, isopropyl, butyl, sec-butyl or tert-butyl), and in case that two or more groups Rₑ are inserted into the backbone carbon chain of the linear or branched C₂₋₃₀ alkylene group, the two or more groups Rₑ are not directly connected to each other; and wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), hydroxy, amino, mercapto, halogen (e.g., fluorine, chlorine, bromine, or iodine), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—), cyano or any combination thereof; or
   when W represents hydrogen, C₂₋₆ alkenyl, C₂₋₆ alkynyl, a leaving group, CHO, COOH, NH₂, hydroxy or mercapto, LIN represents optionally substituted methylene, or optionally substituted linear or branched C₂₋₃₀ alkylene, wherein one or more (e.g., 1-10, 1-8 or 1-6, such as 1, 2, 3, 4, 5 or 6) groups Rₑ and/or one or more (e.g., 1-10, 1-8 or 1-6, such as 1, 2, 3, 4, 5 or 6) groups R_{f} and/or any combination of one or more (e.g., 1-10, 1-8 or 1-6, such as 1, 2, 3, 4, 5 or 6) groups Rₑ and R_{f} are optionally inserted into the backbone carbon chain of the linear or branched C₂₋₃₀ alkylene group, wherein carbon-carbon bond between one or more (e.g., 1-10, 1-8 or 1-6, such as 1, 2, 3, 4, 5 or 6) pairs of adjacent carbon atoms in the backbone carbon chain is interrupted by the group Rₑ, R_{f}, or a combination of Rₑ and R_{f}; wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl (e.g., C₁₋₄ alkyl, such as methyl, CD₃, ethyl, propyl, isopropyl, butyl, sec-butyl or tert-butyl), and in case that two or more groups Rₑ are inserted into the backbone carbon chain of the linear or branched C₂₋₃₀ alkylene group, the two or more groups Rₑ are not directly connected to each other; and wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₃₋₆ cycloalkyl (e.g., optionally perdeuterated cyclopropyl, optionally perdeuterated cyclobutyl, optionally perdeuterated cyclopentyl, or optionally perdeuterated cyclohexyl), hydroxy, amino, mercapto, halogen (e.g., fluorine, chlorine, bromine, or iodine), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH— (e.g., optionally deuterated C₁₋₃ alkyl-NH—, such as CH₃NH—, CH₃CH₂NH— or CH₃CH₂CH₂NH—), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), NH₂—C₁₋₆ alkylene (e.g., NH₂—C₁₋₃ alkylene-, such as NH₂CH₂—, NH₂CH₂CH₂— and NH₂CH₂CH₂CH₂—), optionally deuterated C₁₋₆ alkyl-NHC(O)— (e.g., optionally deuterated C₁₋₄ alkyl-NHC(O)—, such as CH₃—NHC(O)—, CH₃CH₂—NHC(O)—, and CH₃CH₂CH₂—NHC(O)—), optionally deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—), cyano or any combination thereof; or
in case that the compound of Formula (II) is not deuterated, LIN represents optionally substituted methylene, and W represents optionally substituted nitrogen-containing monocyclic heterocyclyl or optionally substituted nitrogen-containing bridged heterocyclyl; and
wherein the nitrogen-containing monocyclic heterocyclylene, the nitrogen-containing bridged heterocyclylene, the nitrogen-containing monocyclic heterocyclyl, the nitrogen-containing bridged heterocyclyl, the cycloalkyl, the heteroaryl and the aryl are each independently optionally substituted with one or more (e.g., 1-10, 1-8, or 1-6, such as 1, 2, 3, 4, 5 or 6) substituents selected from the group consisting of deuterium, hydroxy, amino, mercapto, nitro, halogen (e.g., fluorine, chlorine, bromine, or iodine), cyano, CHO, COOH, N₃, methanesulfonyloxy, trifluoromethanesulfonyloxy, p-toluenesulfonyloxy, C₁₋₆ alkyl (e.g., C₁₋₄ alkyl, such as methyl, ethyl, propyl, isopropyl, butyl, sec-butyl or tert-butyl), deuterated C₁₋₆ alkyl (e.g., perdeuterated C₁₋₄ alkyl, such as CD₃, perdeuterated ethyl, perdeuterated propyl, perdeuterated isopropyl, perdeuterated butyl, perdeuterated sec-butyl or perdeuterated tert-butyl), halogenated C₁₋₆ alkyl, optionally substituted C₃₋₈ cycloalkyl (e.g., substituted with one or more, e.g., 1-10, 1-8 or 1-6, such as 1, 2, 3, 4, 5 or 6 substituents selected from the group consisting of deuterium, halogen, amino, hydroxy, mercapto, cyano, CHO, COOH, N₃, methanesulfonyloxy, trifluoromethanesulfonyloxy and p-toluenesulfonyloxy), optionally substituted 4- to 9-membered heterocyclyl (e.g., substituted with one or more substituents selected from the group consisting of deuterium, halogen, amino, hydroxy, mercapto, cyano, CHO, COOH, N₃, methanesulfonyloxy, trifluoromethanesulfonyloxy and p-toluenesulfonyloxy), C₁₋₆ alkoxy (e.g., C₁₋₄ alkoxy, such as methoxy, CD₃-O—, ethoxy, propoxy, isopropoxy, butoxy, sec-butoxy or tert-butoxy), deuterated C₁₋₆ alkyl-O—, C₁₋₆ alkyl-NH— (e.g., C₁₋₃ alkyl-NH—, such as CH₃NH—, CH₃CH₂NH— or CH₃CH₂CH₂NH—), deuterated C₁₋₆ alkyl-NH— (e.g., perdeuterated C₁₋₃ alkyl-NH—, such as CD₃NH—, CD₃CD₂NH— or CD₃CD₂CD₂NH—), NH₂—C₁₋₆ alkylene (e.g., NH₂—C₁₋₃ alkylene-, such as NH₂CH₂—, NH₂CH₂CH₂— and NH₂CH₂CH₂CH₂—), C₁₋₆ alkyl-NHC(O)— (e.g., C₁₋₄ alkyl-NHC(O)—, such as CH₃—NHC(O)—, CH₃CH₂—NHC(O)—, and CH₃CH₂CH₂—NHC(O)—), deuterated C₁₋₆ alkyl-NHC(O)— (e.g., perdeuterated C₁₋₄ alkyl-NHC(O)—, such as CD₃-NHC(O)—, CD₃CD₂-NHC(O)—, and CD₃CD₂CD₂-NHC(O)—), C₁₋₆ alkyl-C(O)NH—, deuterated C₁₋₆ alkyl-C(O)NH— (e.g., optionally deuterated C₁₋₄ alkyl-C(O)NH—, such as CH₃—C(O)NH—, CH₃CH₂—C(O)NH—, and CH₃CH₂CH₂—C(O)NH—) or any combination thereof.

In some embodiments, the compounds of Formula (II) of the present invention and their salts (including pharmaceutically acceptable salts, such as hydrochloride, etc.), prodrugs, solvates, isotopically enriched analogs, polymorphs, stereoisomers (including enantiomers and diastereomers), or mixture of stereoisomers thereof in Table 2 below are provided.

TABLE 2
The compounds of the present disclosure

| Com- | | |
|---|---|---|
| pound | | |
| No. | Structure of compound | Compound's name |
| | 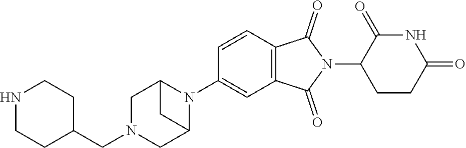 [SMILES: O=C1CCC(N2C(=O)c3ccc(N4C5CC4CN(CC4CCNCC4)C5)cc3C2=O)C(=O)N1] | 2-(2,6-dioxopiperidin-3-yl)-5-(3-(piperidin-4-ylmethyl)- 3,6-diazabicyclo[3.1.1]heptan-6-yl)isoindoline-1,3-dione |
| | 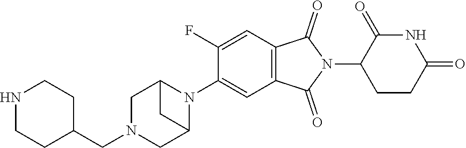 [SMILES: O=C1CCC(N2C(=O)c3cc(F)c(N4C5CC4CN(CC4CCNCC4)C5)cc3C2=O)C(=O)N1] | 2-(2,6-dioxopiperidin-3-yl)-5-fluoro-6-(3-(piperidin-4- ylmethyl)-3,6-diazabicyclo[3.1.1]heptan-6-yl)isoindoline- 1,3-dione |
| | 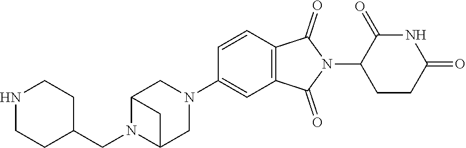 [SMILES: O=C1CCC(N2C(=O)c3ccc(N4CC5CC(C4)N5CC4CCNCC4)cc3C2=O)C(=O)N1] | 2-(2,6-dioxopiperidin-3-yl)-5-(6-(piperidin-4-ylmethyl)- 3,6-diazabicyclo[3.1.1]heptan-3-yl)isoindoline-1,3-dione |
| | 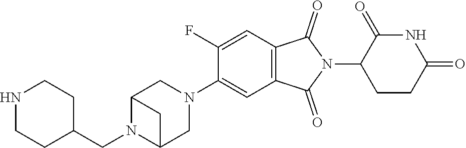 [SMILES: O=C1CCC(N2C(=O)c3cc(F)c(N4CC5CC(C4)N5CC4CCNCC4)cc3C2=O)C(=O)N1] | 2-(2,6-dioxopiperidin-3-yl)-5-fluoro-6-(6-(piperidin-4- ylmethyl)-3,6-diazabicyclo[3.1.1]heptan-3-yl)isoindoline- 1,3-dione |
| | 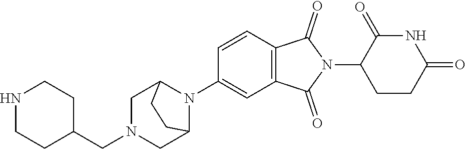 [SMILES: O=C1CCC(N2C(=O)c3ccc(N4C5CCC4CN(CC4CCNCC4)C5)cc3C2=O)C(=O)N1] | 2-(2,6-dioxopiperidin-3-yl)-5-(3-(piperidin-4-ylmethyl)- 3,8-diazabicyclo[3.2.1]octan-8-yl)isoindoline-1,3-dione |
| | 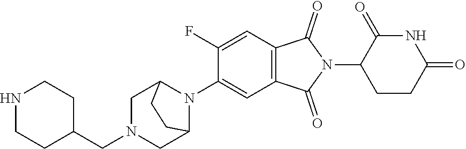 [SMILES: O=C1CCC(N2C(=O)c3cc(F)c(N4C5CCC4CN(CC4CCNCC4)C5)cc3C2=O)C(=O)N1] | 2-(2,6-dioxopiperidin-3-yl)-5-fluoro-6-(3-(piperidin-4- ylmethyl)-3,8-diazabicyclo[3.2.1]octan-8-yl)isoindoline- 1,3-dione |
| | 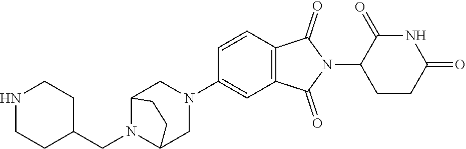 [SMILES: O=C1CCC(N2C(=O)c3ccc(N4CC5CCC(C4)N5CC4CCNCC4)cc3C2=O)C(=O)N1] | 2-(2,6-dioxopiperidin-3-yl)-5-(8-(piperidin-4-ylmethyl)- 3,8-diazabicyclo[3.2.1]octan-3-yl)isoindoline-1,3-dione |
| | 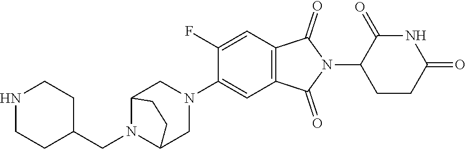 [SMILES: O=C1CCC(N2C(=O)c3cc(F)c(N4CC5CCC(C4)N5CC4CCNCC4)cc3C2=O)C(=O)N1] | 2-(2,6-dioxopiperidin-3-yl)-5-fluoro-6-(8-(piperidin-4- ylmethyl)-3,8-diazabicyclo[3.2.1]octan-3-yl)isoindoline- 1,3-dione |
| | 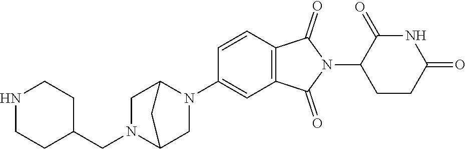 [SMILES: O=C1CCC(N2C(=O)c3ccc(N4CC5CC4CN5CC4CCNCC4)cc3C2=O)C(=O)N1] | 2-(2,6-dioxopiperidin-3-yl)-5-(5-(piperidin-4-ylmethyl)- 2,5-diazabicyclo[2.2.1]heptan-2-yl)isoindoline-1,3-dione |
| | 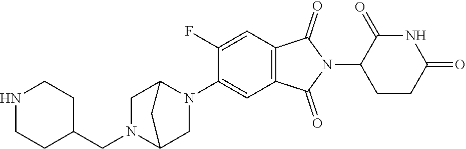 [SMILES: O=C1CCC(N2C(=O)c3cc(F)c(N4CC5CC4CN5CC4CCNCC4)cc3C2=O)C(=O)N1] | 2-(2,6-dioxopiperidin-3-yl)-5-fluoro-6-(5-(piperidin-4- ylmethyl)-2,5-diazabicyclo[2.2.1]heptan-2-yl)isoindoline- 1,3-dione |
| | 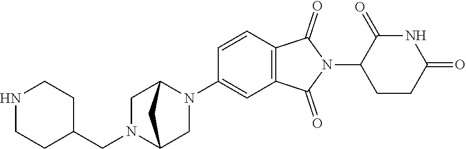 [SMILES: O=C1CCC(N2C(=O)c3ccc(N4C[C@H]5C[C@@H]4CN5CC4CCNCC4)cc3C2=O)C(=O)N1] | 2-(2,6-dioxopiperidin-3-yl)-5-((1R,4R)-5-(piperidin-4- ylmethyl)-2,5-diazabicyclo[2.2.1]heptan-2-yl)isoindoline- 1,3-dione |
| | 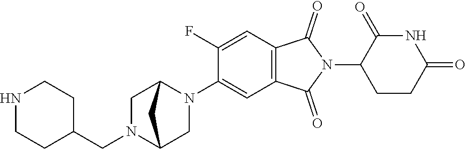 [SMILES: O=C1CCC(N2C(=O)c3cc(F)c(N4C[C@H]5C[C@@H]4CN5CC4CCNCC4)cc3C2=O)C(=O)N1] | 2-(2,6-dioxopiperidin-3-yl)-5-fluoro-6-((1R,4R)-5- (piperidin-4-ylmethyl)-2,5-diazabicyclo[2.2.1]heptan-2- yl)isoindoline-1,3-dione |
| | 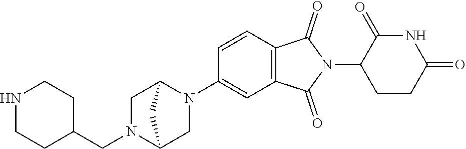 [SMILES: O=C1CCC(N2C(=O)c3ccc(N4C[C@@H]5C[C@H]4CN5CC4CCNCC4)cc3C2=O)C(=O)N1] | 2-(2,6-dioxopiperidin-3-yl)-5-((1S,4S)-5-(piperidin-4- ylmethyl)-2,5-diazabicyclo[2.2.1]heptan-2-yl)isoindoline- 1,3-dione |
| | 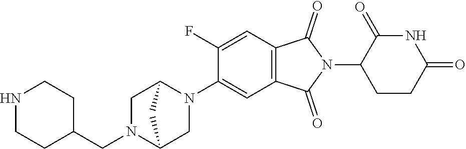 [SMILES: O=C1CCC(N2C(=O)c3cc(F)c(N4C[C@@H]5C[C@H]4CN5CC4CCNCC4)cc3C2=O)C(=O)N1] | 2-(2,6-dioxopiperidin-3-yl)-5-fluoro-6-((1S,4S)-5- (piperidin-4-ylmethyl)-2,5-diazabicyclo[2.2.1]heptan-2- yl)isoindoline-1,3-dione |
| | 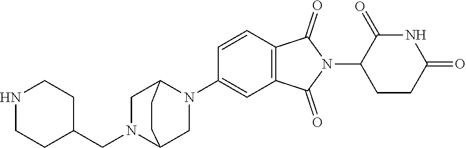 [SMILES: O=C1CCC(N2C(=O)c3ccc(N4CC5CCC4CN5CC4CCNCC4)cc3C2=O)C(=O)N1] | 2-(2,6-dioxopiperidin-3-yl)-5-(5-(piperidin-4-ylmethyl)- 2,5-diazabicyclo[2.2.2]octan-2-yl)isoindoline-1,3-dione |
| | 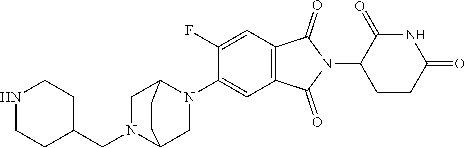 [SMILES: O=C1CCC(N2C(=O)c3cc(F)c(N4CC5CCC4CN5CC4CCNCC4)cc3C2=O)C(=O)N1] | 2-(2,6-dioxopiperidin-3-yl)-5-fluoro-6-(5-(piperidin-4- ylmethyl)-2,5-diazabicyclo[2.2.2]octan-2-yl)isoindoline- 1,3-dione |
| | 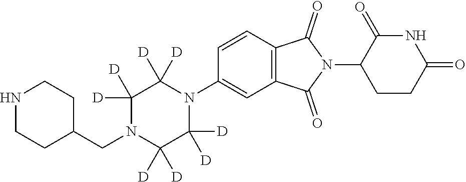 [SMILES: [2H]C1([2H])N(CC2CCNCC2)C([2H])([2H])C([2H])([2H])N(c2ccc3c(c2)C(=O)N(C2CCC(=O)NC2=O)C3=O)C1([2H])[2H]] | 2-(2,6-dioxopiperidin-3-yl)-5-(4-(piperidin-4- ylmethyl)piperazin-1-yl-2,2,3,3,5,5,6,6-d8)isoindoline- 1,3-dione |
| | 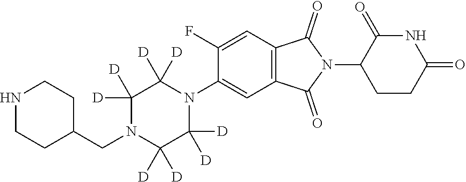 [SMILES: [2H]C1([2H])N(CC2CCNCC2)C([2H])([2H])C([2H])([2H])N(c2cc3c(cc2F)C(=O)N(C2CCC(=O)NC2=O)C3=O)C1([2H])[2H]] | 2-(2,6-dioxopiperidin-3-yl)-5-fluoro-6-(4-(piperidin-4- ylmethyl)piperazin-1-yl-2,2,3,3,5,5,6,6-d8)isoindoline- 1,3-dione |
| | 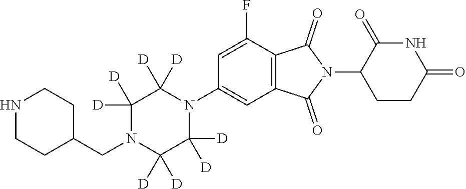 [SMILES: [2H]C1([2H])N(CC2CCNCC2)C([2H])([2H])C([2H])([2H])N(c2cc(F)c3c(c2)C(=O)N(C2CCC(=O)NC2=O)C3=O)C1([2H])[2H]] | 2-(2,6-dioxopiperidin-3-yl)-4-fluoro-6-(4-(piperidin-4- ylmethyl)piperazin-1-yl-2,2,3,3,5,5,6,6-d8)isoindoline- 1,3-dione |
| | 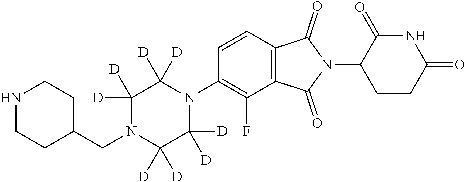 [SMILES: [2H]C1([2H])N(CC2CCNCC2)C([2H])([2H])C([2H])([2H])N(c2ccc3c(c2F)C(=O)N(C2CCC(=O)NC2=O)C3=O)C1([2H])[2H]] | 2-(2,6-dioxopiperidin-3-yl)-4-fluoro-5-(4-(piperidin-4- ylmethyl)piperazin-1-yl-2,2,3,3,5,5,6,6-d8)isoindoline- 1,3-dione |
| | 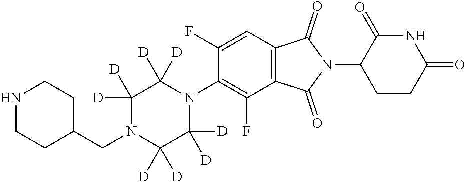 [SMILES: [2H]C1([2H])N(CC2CCNCC2)C([2H])([2H])C([2H])([2H])N(c2c(F)cc3c(c2F)C(=O)N(C2CCC(=O)NC2=O)C3=O)C1([2H])[2H]] | 2-(2,6-dioxopiperidin-3-yl)-4,6-difluoro-5-(4-(piperidin- 4-ylmethyl)piperazin-1-yl-2,2,3,3,5,5,6,6-d8)isoindoline- 1,3-dione |
| | 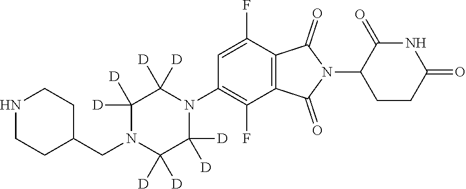 [SMILES: [2H]C1([2H])N(CC2CCNCC2)C([2H])([2H])C([2H])([2H])N(c2cc(F)c3c(c2F)C(=O)N(C2CCC(=O)NC2=O)C3=O)C1([2H])[2H]] | 2-(2,6-dioxopiperidin-3-yl)-4,7-difluoro-5-(4-(piperidin- 4-ylmethyl)piperazin-1-yl-2,2,3,3,5,5,6,6-d8)isoindoline- 1,3-dione |
| | 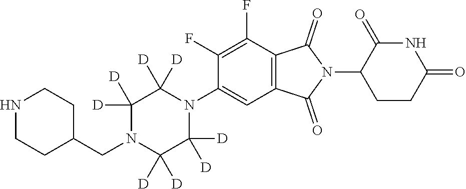 [SMILES: [2H]C1([2H])N(CC2CCNCC2)C([2H])([2H])C([2H])([2H])N(c2cc3c(c(F)c2F)C(=O)N(C2CCC(=O)NC2=O)C3=O)C1([2H])[2H]] | 2-(2,6-dioxopiperidin-3-yl)-4,5-difluoro-6-(4-(piperidin- 4-ylmethyl)piperazin-1-yl-2,2,3,3,5,5,6,6-d8)isoindoline- 1,3-dione |
| | 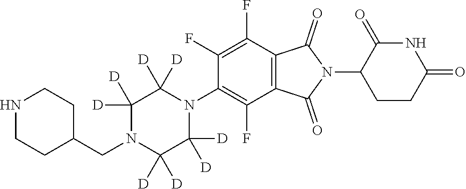 [SMILES: [2H]C1([2H])N(CC2CCNCC2)C([2H])([2H])C([2H])([2H])N(c2c(F)c(F)c3c(c2F)C(=O)N(C2CCC(=O)NC2=O)C3=O)C1([2H])[2H]] | 2-(2,6-dioxopiperidin-3-yl)-4,5,7-trifluoro-6-(4- (piperidin-4-ylmethyl)piperazin-1-yl-2,2,3,3,5,5,6,6- d8)isoindoline-1,3-dione |
| | 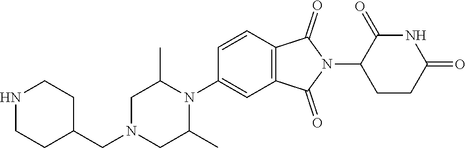 [SMILES: CC1CN(CC2CCNCC2)CC(C)N1c1ccc2c(c1)C(=O)N(C1CCC(=O)NC1=O)C2=O] | 5-(2,6-dimethyl-4-(piperidin-4-ylmethyl)piperazin-1-yl)- 2-(2,6-dioxopiperidin-3-yl)isoindoline-1,3-dione |
| | 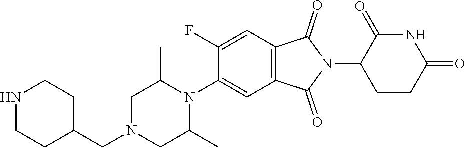 [SMILES: CC1CN(CC2CCNCC2)CC(C)N1c1cc2c(cc1F)C(=O)N(C1CCC(=O)NC1=O)C2=O] | 5-(2,6-dimethyl-4-(piperidin-4-ylmethyl)piperazin-1-yl)- 2-(2,6-dioxopiperidin-3-yl)-6-fluoroisoindoline-1,3-dione |
| | 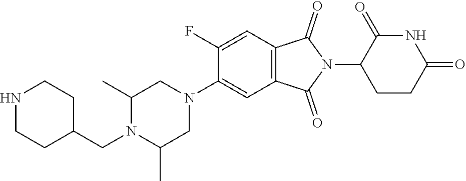 [SMILES: CC1CN(c2cc3c(cc2F)C(=O)N(C2CCC(=O)NC2=O)C3=O)CC(C)N1CC1CCNCC1] | 5-(3,5-dimethyl-4-(piperidin-4-ylmethyl)piperazin-1-yl)- 2-(2,6-dioxopiperidin-3-yl)-6-fluoroisoindoline-1,3-dione |
| | 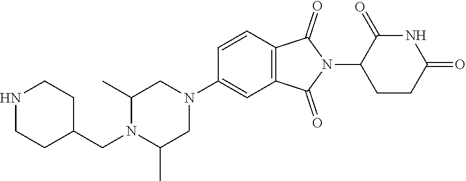 [SMILES: CC1CN(c2ccc3c(c2)C(=O)N(C2CCC(=O)NC2=O)C3=O)CC(C)N1CC1CCNCC1] | 5-(3,5-dimethyl-4-(piperidin-4-ylmethyl)piperazin-1-yl)- 2-(2,6-dioxopiperidin-3-yl)isoindoline-1,3-dione |
| | 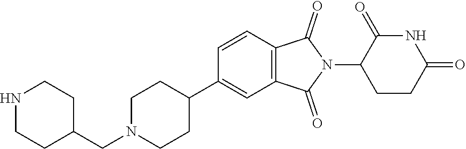 [SMILES: O=C1CCC(N2C(=O)c3ccc(C4CCN(CC5CCNCC5)CC4)cc3C2=O)C(=O)N1] | 2-(2,6-dioxopiperidin-3-yl)-5-(1-(piperidin-4- ylmethyl)piperidin-4-yl)isoindoline-1,3-dione |
| | 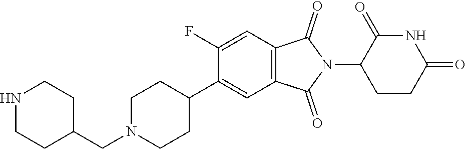 [SMILES: O=C1CCC(N2C(=O)c3cc(F)c(C4CCN(CC5CCNCC5)CC4)cc3C2=O)C(=O)N1] | 2-(2,6-dioxopiperidin-3-yl)-5-fluoro-6-(1-(piperidin-4- ylmethyl)piperidin-4-yl)isoindoline-1,3-dione |
| | 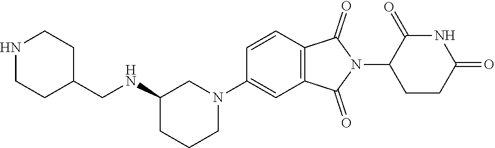 [SMILES: O=C1CCC(N2C(=O)c3ccc(N4CCC[C@@H](NCC5CCNCC5)C4)cc3C2=O)C(=O)N1] | 2-(2,6-dioxopiperidin-3-yl)-5-((R)-3-((piperidin-4- ylmethyl)amino)piperidin-1-yl)isoindoline-1,3-dione |
| | 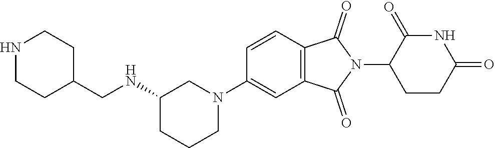 [SMILES: O=C1CCC(N2C(=O)c3ccc(N4CCC[C@H](NCC5CCNCC5)C4)cc3C2=O)C(=O)N1] | 2-(2,6-dioxopiperidin-3-yl)-5-((S)-3-((piperidin-4- ylmethyl)amino)piperidin-1-yl)isoindoline-1,3-dione |
| | 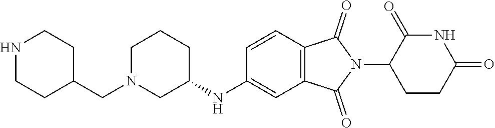 [SMILES: O=C1CCC(N2C(=O)c3ccc(N[C@H]4CCCN(CC5CCNCC5)C4)cc3C2=O)C(=O)N1] | 2-(2,6-dioxopiperidin-3-yl)-5-(((S)-1-(piperidin-4- ylmethyl)piperidin-3-yl)amino)isoindoline-1,3-dione |
| | 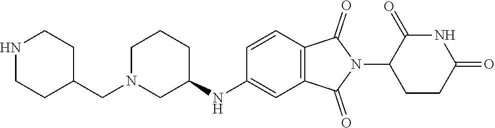 [SMILES: O=C1CCC(N2C(=O)c3ccc(N[C@@H]4CCCN(CC5CCNCC5)C4)cc3C2=O)C(=O)N1] | 2-(2,6-dioxopiperidin-3-yl)-5-(((R)-1-(piperidin-4- ylmethyl)piperidin-3-yl)amino)isoindoline-1,3-dione |
| | 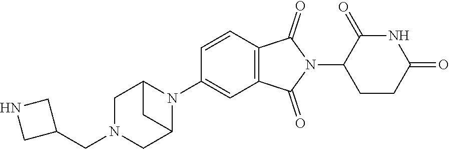 [SMILES: O=C1CCC(N2C(=O)c3ccc(N4C5CC4CN(CC4CNC4)C5)cc3C2=O)C(=O)N1] | 5-(3-(azetidin-3-ylmethyl)-3,6-diazabicyclo[3.1.1]heptan- 6-yl)-2-(2,6-dioxopiperidin-3-yl)isoindoline-1,3-dione |
| | 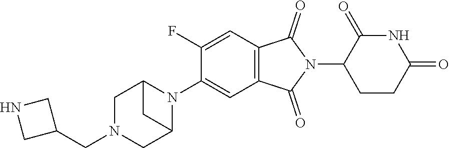 [SMILES: O=C1CCC(N2C(=O)c3cc(F)c(N4C5CC4CN(CC4CNC4)C5)cc3C2=O)C(=O)N1] | 5-(3-(azetidin-3-ylmethyl)-3,6-diazabicyclo[3.1.1]heptan- 6-yl)-2-(2,6-dioxopiperidin-3-yl)-6-fluoroisoindoline-1,3- dione |
| | 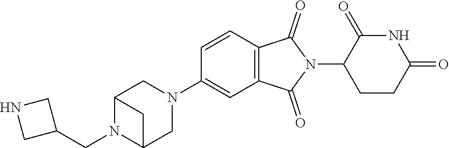 [SMILES: O=C1CCC(N2C(=O)c3ccc(N4CC5CC(C4)N5CC4CNC4)cc3C2=O)C(=O)N1] | 5-(6-(azetidin-3-ylmethyl)-3,6-diazabicyclo[3.1.1]heptan- 3-yl)-2-(2,6-dioxopiperidin-3-yl)isoindoline-1,3-dione |
| | 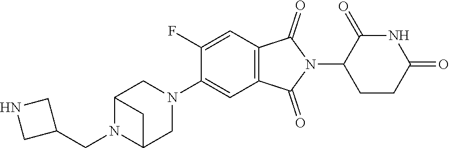 [SMILES: O=C1CCC(N2C(=O)c3cc(F)c(N4CC5CC(C4)N5CC4CNC4)cc3C2=O)C(=O)N1] | 5-(6-(azetidin-3-ylmethyl)-3,6-diazabicyclo[3.1.1]heptan- 3-yl)-2-(2,6-dioxopiperidin-3-yl)-6-fluoroisoindoline-1,3- dione |
| | 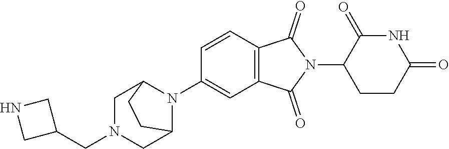 [SMILES: O=C1CCC(N2C(=O)c3ccc(N4C5CCC4CN(CC4CNC4)C5)cc3C2=O)C(=O)N1] | 5-(3-(azetidin-3-ylmethyl)-3,8-diazabicyclo[3.2.1]octan- 8-yl)-2-(2,6-dioxopiperidin-3-yl)isoindoline-1,3-dione |
| | 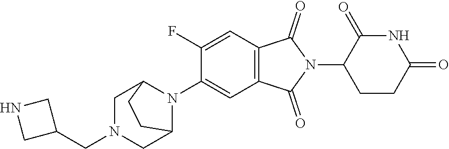 [SMILES: O=C1CCC(N2C(=O)c3cc(F)c(N4C5CCC4CN(CC4CNC4)C5)cc3C2=O)C(=O)N1] | 5-(3-(azetidin-3-ylmethyl)-3,8-diazabicyclo[3.2.1]octan- 8-yl)-2-(2,6-dioxopiperidin-3-yl)-6-fluoroisoindoline-1,3- dione |
| | 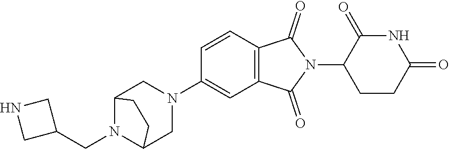 [SMILES: O=C1CCC(N2C(=O)c3ccc(N4CC5CCC(C4)N5CC4CNC4)cc3C2=O)C(=O)N1] | 5-(8-(azetidin-3-ylmethyl)-3,8-diazabicyclo[3.2.1]octan- 3-yl)-2-(2,6-dioxopiperidin-3-yl)isoindoline-1,3-dione |
| | 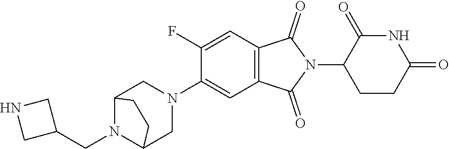 [SMILES: O=C1CCC(N2C(=O)c3cc(F)c(N4CC5CCC(C4)N5CC4CNC4)cc3C2=O)C(=O)N1] | 2-(2,6-dioxopiperidin-3-yl)-5-fluoro-6-(8-(piperidin-4- ylmethyl)-3,8-diazabicyclo[3.2.1]octan-3-yl)isoindoline- 1,3-dione |
| | 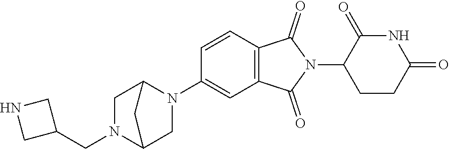 [SMILES: O=C1CCC(N2C(=O)c3ccc(N4CC5CC4CN5CC4CNC4)cc3C2=O)C(=O)N1] | 5-(5-(azetidin-3-ylmethyl)-2,5-diazabicyclo[2.2.1]heptan- 2-yl)-2-(2,6-dioxopiperidin-3-yl)isoindoline-1,3-dione |
| | 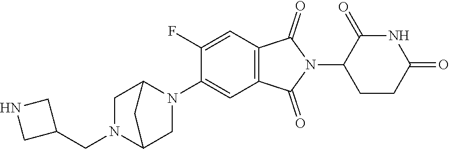 [SMILES: O=C1CCC(N2C(=O)c3cc(F)c(N4CC5CC4CN5CC4CNC4)cc3C2=O)C(=O)N1] | 5-(5-(azetidin-3-ylmethyl)-2,5-diazabicyclo[2.2.1]heptan- 2-yl)-2-(2,6-dioxopiperidin-3-yl)-6-fluoroisoindoline-1,3- dione |
| | 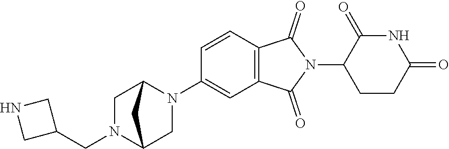 [SMILES: O=C1CCC(N2C(=O)c3ccc(N4C[C@H]5C[C@@H]4CN5CC4CNC4)cc3C2=O)C(=O)N1] | 5-((1R,4R)-5-(azetidin-3-ylmethyl)-2,5- diazabicyclo[2.2.1]heptan-2-yl)-2-(2,6-dioxopiperidin-3- yl)isoindoline-1,3-dione |
| | 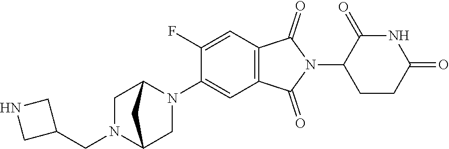 [SMILES: O=C1CCC(N2C(=O)c3cc(F)c(N4C[C@H]5C[C@@H]4CN5CC4CNC4)cc3C2=O)C(=O)N1] | 5-((1R,4R)-5-(azetidin-3-ylmethyl)-2,5- diazabicyclo[2.2.1]heptan-2-yl)-2-(2,6-dioxopiperidin-3- yl)-6-fluoroisoindoline-1,3-dione |
| | 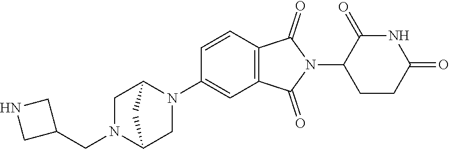 [SMILES: O=C1CCC(N2C(=O)c3ccc(N4C[C@@H]5C[C@H]4CN5CC4CNC4)cc3C2=O)C(=O)N1] | 5-((1S,4S)-5-(azetidin-3-ylmethyl)-2,5- diazabicyclo[2.2.1]heptan-2-yl)-2-(2,6-dioxopiperidin-3- yl)isoindoline-1,3-dione |
| | 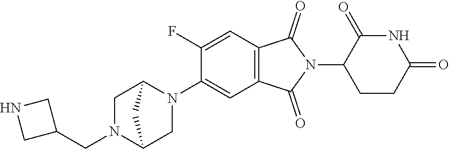 [SMILES: O=C1CCC(N2C(=O)c3cc(F)c(N4C[C@@H]5C[C@H]4CN5CC4CNC4)cc3C2=O)C(=O)N1] | 5-((1S,4S)-5-(azetidin-3-ylmethyl)-2,5- diazabicyclo[2.2.1]heptan-2-yl)-2-(2,6-dioxopiperidin-3- yl)-6-fluoroisoindoline-1,3-dione |
| | 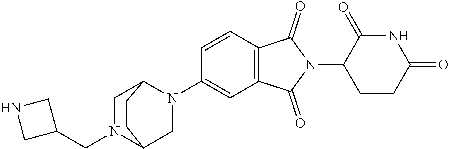 [SMILES: O=C1CCC(N2C(=O)c3ccc(N4CC5CCC4CN5CC4CNC4)cc3C2=O)C(=O)N1] | 5-(5-(azetidin-3-ylmethyl)-2,5-diazabicyclo[2.2.2]octan- 2-yl)-2-(2,6-dioxopiperidin-3-yl)isoindoline-1,3-dione |
| | 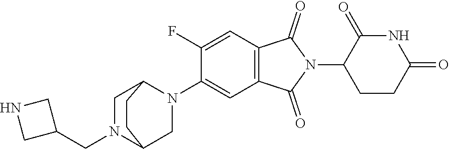 [SMILES: O=C1CCC(N2C(=O)c3cc(F)c(N4CC5CCC4CN5CC4CNC4)cc3C2=O)C(=O)N1] | 5-(5-(azetidin-3-ylmethyl)-2,5-diazabicyclo[2.2.2]octan- 2-yl)-2-(2,6-dioxopiperidin-3-yl)-6-fluoroisoindoline-1,3- dione |
| | 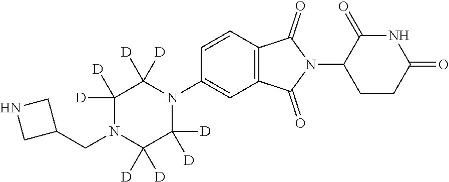 [SMILES: [2H]C1([2H])N(CC2CNC2)C([2H])([2H])C([2H])([2H])N(c2ccc3c(c2)C(=O)N(C2CCC(=O)NC2=O)C3=O)C1([2H])[2H]] | 5-(4-(azetidin-3-ylmethyl)piperazin-1-yl-2,2,3,3,5,5,6,6- d8)-2-(2,6-dioxopiperidin-3-yl)isoindoline-1,3-dione |
| | 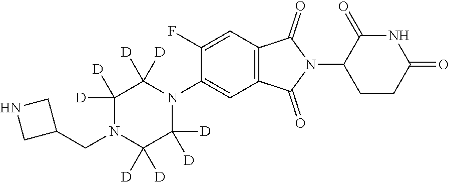 [SMILES: [2H]C1([2H])N(CC2CNC2)C([2H])([2H])C([2H])([2H])N(c2cc3c(cc2F)C(=O)N(C2CCC(=O)NC2=O)C3=O)C1([2H])[2H]] | 5-(4-(azetidin-3-ylmethyl)piperazin-1-yl-2,2,3,3,5,5,6,6- d8)-2-(2,6-dioxopiperidin-3-yl)-6-fluoroisoindoline-1,3- dione |
| | 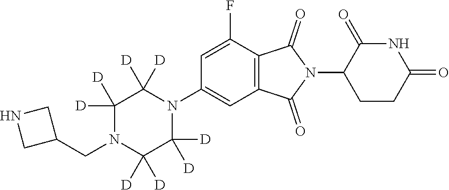 [SMILES: [2H]C1([2H])N(CC2CNC2)C([2H])([2H])C([2H])([2H])N(c2cc(F)c3c(c2)C(=O)N(C2CCC(=O)NC2=O)C3=O)C1([2H])[2H]] | 6-(4-(azetidin-3-ylmethyl)piperazin-1-yl-2,2,3,3,5,5,6,6- d8)-2-(2,6-dioxopiperidin-3-yl)-4-fluoroisoindoline-1,3- dione |
| | 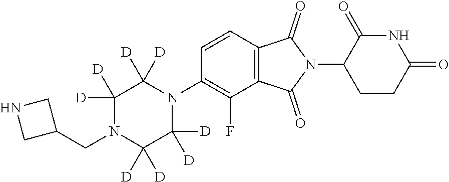 [SMILES: [2H]C1([2H])N(CC2CNC2)C([2H])([2H])C([2H])([2H])N(c2ccc3c(c2F)C(=O)N(C2CCC(=O)NC2=O)C3=O)C1([2H])[2H]] | 5-(4-(azetidin-3-ylmethyl)piperazin-1-yl-2,2,3,3,5,5,6,6- d8)-2-(2,6-dioxopiperidin-3-yl)-4-fluoroisoindoline-1,3- dione |
| | 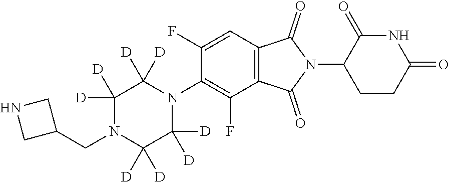 [SMILES: [2H]C1([2H])N(CC2CNC2)C([2H])([2H])C([2H])([2H])N(c2c(F)cc3c(c2F)C(=O)N(C2CCC(=O)NC2=O)C3=O)C1([2H])[2H]] | 5-(4-(azetidin-3-ylmethyl)piperazin-1-yl-2,2,3,3,5,5,6,6- d8)-2-(2,6-dioxopiperidin-3-yl)-4,6-difluoroisoindoline- 1,3-dione |
| | 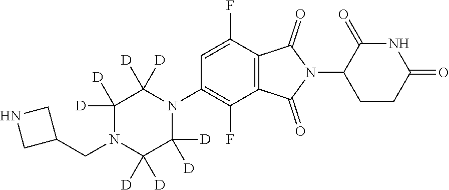 [SMILES: [2H]C1([2H])N(CC2CNC2)C([2H])([2H])C([2H])([2H])N(c2cc(F)c3c(c2F)C(=O)N(C2CCC(=O)NC2=O)C3=O)C1([2H])[2H]] | 5-(4-(azetidin-3-ylmethyl)piperazin-1-yl-2,2,3,3,5,5,6,6- d8)-2-(2,6-dioxopiperidin-3-yl)-4,7-difluoroisoindoline- 1,3-dione |
| | 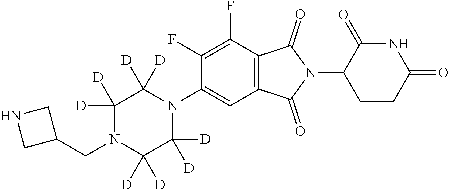 [SMILES: [2H]C1([2H])N(CC2CNC2)C([2H])([2H])C([2H])([2H])N(c2cc3c(c(F)c2F)C(=O)N(C2CCC(=O)NC2=O)C3=O)C1([2H])[2H]] | 6-(4-(azetidin-3-ylmethyl)piperazin-1-yl-2,2,3,3,5,5,6,6- d8)-2-(2,6-dioxopiperidin-3-yl)-4,5-difluoroisoindoline- 1,3-dione |
| | 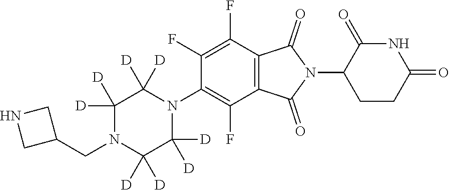 [SMILES: [2H]C1([2H])N(CC2CNC2)C([2H])([2H])C([2H])([2H])N(c2c(F)c(F)c3c(c2F)C(=O)N(C2CCC(=O)NC2=O)C3=O)C1([2H])[2H]] | 5-(4-(azetidin-3-ylmethyl)piperazin-1-yl-2,2,3,3,5,5,6,6- d8)-2-(2,6-dioxopiperidin-3-yl)-4,6,7- trifluoroisoindoline-1,3-dione |
| | 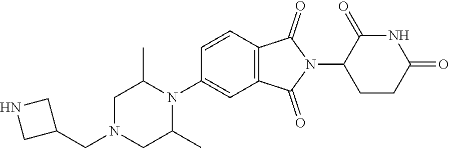 [SMILES: CC1CN(CC2CNC2)CC(C)N1c1ccc2c(c1)C(=O)N(C1CCC(=O)NC1=O)C2=O] | 5-(4-(azetidin-3-ylmethyl)-2,6-dimethylpiperazin-1-yl)-2- (2,6-dioxopiperidin-3-yl)isoindoline-1,3-dione |
| | 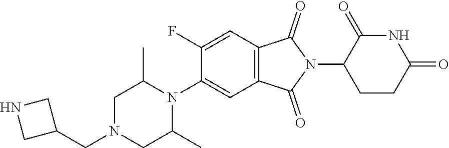 [SMILES: CC1CN(CC2CNC2)CC(C)N1c1cc2c(cc1F)C(=O)N(C1CCC(=O)NC1=O)C2=O] | 5-(4-(azetidin-3-ylmethyl)-2,6-dimethylpiperazin-1-yl)-2- (2,6-dioxopiperidin-3-yl)-6-fluoroisoindoline-1,3-dione |
| | 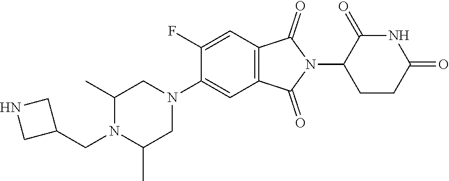 [SMILES: CC1CN(c2cc3c(cc2F)C(=O)N(C2CCC(=O)NC2=O)C3=O)CC(C)N1CC1CNC1] | 5-(4-(azetidin-3-ylmethyl)-3,5-dimethylpiperazin-1-yl)-2- (2,6-dioxopiperidin-3-yl)-6-fluoroisoindoline-1,3-dione |
| | 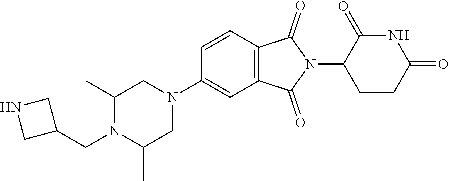 [SMILES: CC1CN(c2ccc3c(c2)C(=O)N(C2CCC(=O)NC2=O)C3=O)CC(C)N1CC1CNC1] | 5-(4-(azetidin-3-ylmethyl)-3,5-dimethylpiperazin-1-yl)-2- (2,6-dioxopiperidin-3-yl)isoindoline-1,3-dione |
| | 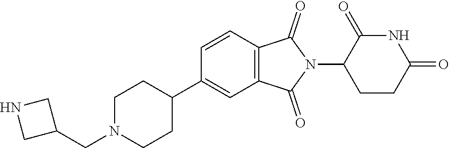 [SMILES: O=C1CCC(N2C(=O)c3ccc(C4CCN(CC5CNC5)CC4)cc3C2=O)C(=O)N1] | 5-(1-(azetidin-3-ylmethyl)piperidin-4-yl)-2-(2,6- dioxopiperidin-3-yl)isoindoline-1,3-dione |
| | 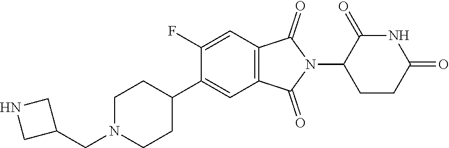 [SMILES: O=C1CCC(N2C(=O)c3cc(F)c(C4CCN(CC5CNC5)CC4)cc3C2=O)C(=O)N1] | 5-(1-(azetidin-3-ylmethyl)piperidin-4-yl)-2-(2,6- dioxopiperidin-3-yl)-6-fluoroisoindoline-1,3-dione |
| | 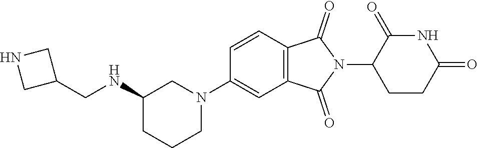 [SMILES: O=C1CCC(N2C(=O)c3ccc(N4CCC[C@@H](NCC5CNC5)C4)cc3C2=O)C(=O)N1] | 5-((R)-3-((azetidin-3-ylmethyl)amino)piperidin-1-yl)-2- (2,6-dioxopiperidin-3-yl)isoindoline-1,3-dione |
| | 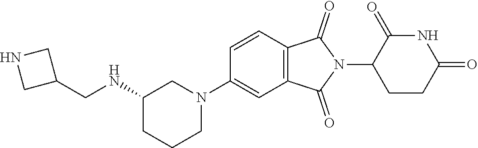 [SMILES: O=C1CCC(N2C(=O)c3ccc(N4CCC[C@H](NCC5CNC5)C4)cc3C2=O)C(=O)N1] | 5-((S)-3-((azetidin-3-ylmethyl)amino)piperidin-1-yl)-2- (2,6-dioxopiperidin-3-yl)isoindoline-1,3-dione |
| | 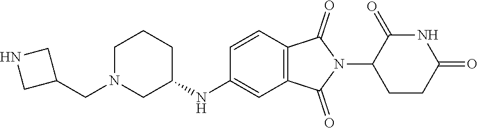 [SMILES: O=C1CCC(N2C(=O)c3ccc(N[C@H]4CCCN(CC5CNC5)C4)cc3C2=O)C(=O)N1] | 5-(((S)-1-(azetidin-3-ylmethyl)piperidin-3-yl)amino)-2- (2,6-dioxopiperidin-3-yl)isoindoline-1,3-dione |
| | 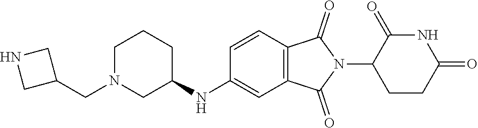 [SMILES: O=C1CCC(N2C(=O)c3ccc(N[C@@H]4CCCN(CC5CNC5)C4)cc3C2=O)C(=O)N1] | 5-(((R)-1-(azetidin-3-ylmethyl)piperidin-3-yl)amino)-2- (2,6-dioxopiperidin-3-yl)isoindoline-1,3-dione |

### Compounds of Formula (III)

The present disclosure provides a compound of Formula (III) or salts (including pharmaceutically acceptable salts), stereoisomers (including enantiomers), solvates, isotopically enriched analogs, prodrugs, or polymorphs thereof:

PBM-R¹—W¹-LIN—ULM Formula (III)

wherein PBM, R¹, W¹, LIN and ULM are as defined in the compounds of Formula (III) above.

When the compound of Formula (III) of the present disclosure is not deuterated, the compound of Formula (III) does not comprise the following compounds of Formula (IV-1):

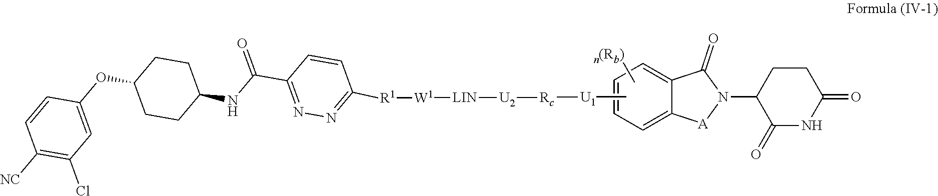

in Formula (IV-1), R¹ represents a bond, W¹ represents piperidinylene, LIN represents methylene, R_{c} represents piperazinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents fluoro, n represents an integer of 1, 2 or 3, and U₁ and U₂ are the same or different and each independently represent a bond, NH or O; or
in Formula (IV-1), R¹ represents a bond, W¹ represents piperazinylene, LIN represents methylene, R_{c} represents piperidinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents fluoro, n represents an integer of 1, 2 or 3, and U₁ and U₂ are the same or different and each independently represent a bond, NH or O.

When the compound of Formula (III) of the present disclosure is not deuterated, the compound of Formula (III) does not comprise the following compound of Formula (IV-2):

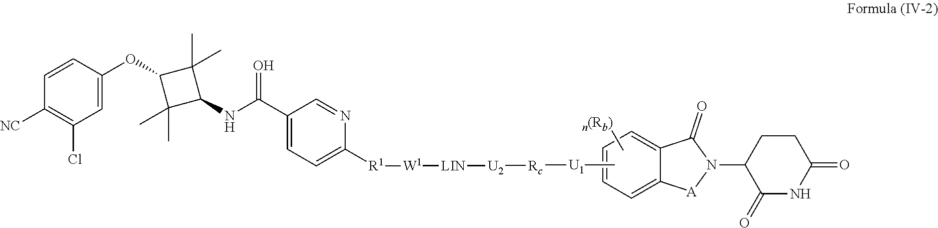

in Formula (IV-2), R¹ represents a bond, W¹ represents piperidinylene, LIN represents methylene, R_{c} represents piperazinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents fluoro, n represents an integer of 1, 2 or 3, and U₁ and U₂ are the same or different and each independently represent a bond, NH or O; or
in Formula (IV-2), R¹ represents a bond, W¹ represents piperazinylene, LIN represents methylene, R_{c} represents piperidinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents fluoro, and n represents an integer of 1, 2 or 3, and U₁ and U₂ are the same or different and each independently represent a bond, NH or O.

When the compound of Formula (III) of the present disclosure is not deuterated, the compound of Formula (III) does not comprise the following compounds of Formula (IV-3):

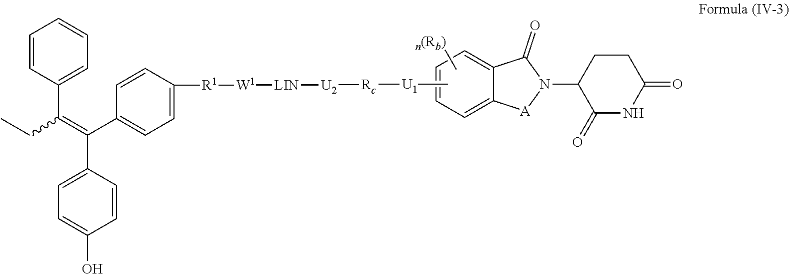

in Formula (IV-3), R¹ represents a bond or —O—CH₂—CH₂—, W¹ represents piperidinylene, LIN represents methylene, R_{c} represents piperazinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents hydrogen, and U₁ and U₂ are the same or different and each independently represent a bond, NH or O; or
in Formula (IV-3), R¹ represents a bond or —O—CH₂—CH₂—, W¹ represents piperazinylene, LIN represents methylene, R_{c} represents piperidinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents hydrogen, and U₁ and U₂ are the same or different and each independently represent a bond, NH or O.

When the compound of Formula (III) of the present disclosure is not deuterated, the compound of Formula (III) does not comprise the following compounds of Formula (IV-4):

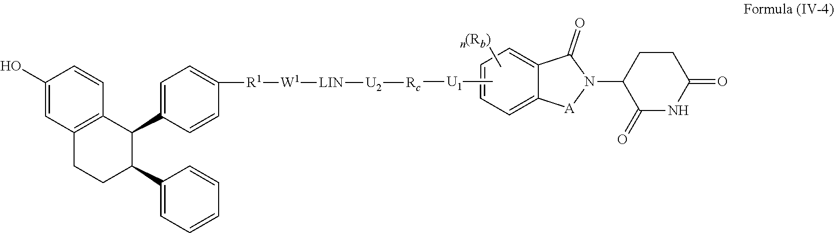

in Formula (IV-4), R¹ represents a bond or represents —O—CH₂—CH₂—, W¹ represents piperidinylene, LIN represents methylene, R_{c} represents piperazinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents hydrogen, and U₁ and U₂ are the same or different and each independently represent a bond, NH or O; or
in Formula (IV-4), R¹ represents a bond or represents —O—CH₂—CH₂—, W¹ represents piperazinylene, LIN represents methylene, R_{c} represents piperidinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents hydrogen, and U and U₂ are the same or different and each independently represent a bond, NH or O.

The compound of Formula (III) or salts (including pharmaceutically acceptable salts), stereoisomers (including enantiomers), solvates, isotope enriched analogues, prodrugs, or polymorphs thereof of the present disclosure can bind to target proteins, recruit target proteins to E3 ubiquitin ligases for ubiquitination labeling and degradation. The compound of Formula (III) of the present disclosure can specifically target specific type or family of target protein, and exhibit a wide range of pharmacological activities by degrading/inhibiting diverse types or families of target proteins.

In some embodiments, specific target protein includes, but is not limited to, the target proteins in Table 3:

TABLE 3
Applicable specific target proteins

| abbreviation of target protein | target protein′s name |
|---|---|
| ESR1 | Estrogen receptor |
| AR | Androgen receptor |
| BTK | Tyrosine-protein kinase BTK |
| IRAK4 | Interleukin-1 receptor-associated kinase 4 |
| EGFR | Epidermal growth factor receptor |
| MET | Hepatocyte growth factor receptor |
| KIT | Mast/stem cell growth factor receptor Kit |
| EPHA2 | Ephrin type-A receptor 2 |
| PDE4D | cAMP-specific 3′,5′-cyclic phosphodiesterase 4D |
| SRC | Proto-oncogene tyrosine-protein kinase Src |
| BRAF | Serine/threonine-protein kinase B-raf |
| FGFR2 | Fibroblast growth factor receptor 2 |
| FGFR1 | Fibroblast growth factor receptor 1 |
| LYN | Tyrosine-protein kinase Lyn |
| ITK | Tyrosine-protein kinase ITK/TSK |
| PARP1 | Poly [ADP-ribose] polymerase 1 |
| HDAC2 | Histone deacetylase 2 |
| HDAC3 | Histone deacetylase 3 |
| JAK1 | Tyrosine-protein kinase JAK1 |
| BCL2 | Apoptosis regulator Bcl-2 |
| HDAC6 | Histone deacetylase 6 |
| CRBN | Protein cereblon |
| EPHB2 | Ephrin type-B receptor 2 |
| BLK | Tyrosine-protein kinase Blk |
| HDAC1 | Histone deacetylase 1 |
| IGF1R | Insulin-like growth factor 1 receptor |
| TGFBR1 | TGF-beta receptor type-1 |
| AKT2 | RAC-beta serine/threonine-protein kinase |
| AKT1 | RAC-alpha serine/threonine-protein kinase |
| FAK | Focal adhesion kinase |
| MAPK1 | Mitogen-activated protein kinase 1 |
| MAPK14 | Mitogen-activated protein kinase 14 |
| CDK9 | Cyclin-dependent kinase 9 |
| MCL1 | Induced myeloid leukemia cell differentiation protein Mcl-1 |
| BRD4 | Bromodomain-containing protein 4 |
| BRD3 | Bromodomain-containing protein 3 |
| CDK13 | Cyclin-dependent kinase 13 |
| BCL2L1 | Bcl-2-like protein 1 |
| CDK12 | Cyclin-dependent kinase 12 |
| CDK1 | Cyclin-dependent kinase 1 |
| AKT3 | RAC-gamma serine/threonine-protein kinase |
| CDK11B | Cyclin-dependent kinase 11B |
| PAK4 | Serine/threonine-protein kinase PAK 4 |
| MAPKAPK2 | MAP kinase-activated protein kinase 2 |
| TNK2 | Activated CDC42 kinase 1 |
| SIRT2 | NAD-dependent protein deacetylase sirtuin-2 |
| DAPK1 | Death-associated protein kinase 1 |
| ABL2 | Tyrosine-protein kinase ABL2 |
| PRKAA2 | 5′-AMP-activated protein kinase catalytic subunit alpha-2 |
| KAT2A | Histone acetyltransferase KAT2A |
| PBRM1 | Protein polybromo-1 |
| EIF2AK2 | Interferon-induced, double-stranded RNA-activated protein kinase |
| MAP3K7 | Mitogen-activated protein kinase kinase kinase 7 |
| MAPT | Microtubule-associated protein tau |
| RIPK1 | Receptor-interacting serine/threonine-protein kinase 1 |
| IRAK1 | Interleukin-1 receptor-associated kinase 1 |
| MAP4K1 | Mitogen-activated protein kinase kinase kinase kinase 1 |
| MARK4 | MAP/microtubule affinity-regulating kinase 4 |
| BRD9 | Bromodomain-containing protein 9 |
| RIPK2 | Receptor-interacting serine/threonine-protein kinase 2 |
| LIMK1 | LIM domain kinase 1 |
| STK38 | Serine/threonine-protein kinase 38 |
| TRIM24 | Transcription intermediary factor 1-alpha |
| SMARCA4 | Transcription activator BRG1 |
| PRKAA1 | 5′-AMP-activated protein kinase catalytic subunit alpha-1 |
| TBK1 | Serine/threonine-protein kinase TBK1 |
| KRAS | GTPase KRas |
| SMARCA2 | Probable global transcription activator SNF2L2 |
| PCNA | Proliferating cell nuclear antigen |
| BRD7 | Bromodomain-containing protein 7 |
| SUZ12 | Polycomb protein SUZ12 |
| IKZF1 | DNA-binding protein Ikaros |
| HTT | Huntingtin |
| SNCA | Alpha-synuclein |
| SLC9A1 | Sodium/hydrogen exchanger 1 |
| FER | Tyrosine-protein kinase Fer |
| MAP4K2 | Mitogen-activated protein kinase kinase kinase kinase 2 |
| DLG4 | Disks large homolog 4 |
| IKZF3 | Zinc finger protein Aiolos |
| PDE4A | cAMP-specific 3′,5′-cyclic phosphodiesterase 4A |
| HMGCR | 3-hydroxy-3-methylglutaryl-coenzyme A reductase |
| ABL1 | Tyrosine-protein kinase ABL1 |
| RARA | Retinoic acid receptor alpha |
| FLT3 | Receptor-type tyrosine-protein kinase FLT3 |
| RARG | Retinoic acid receptor gamma |
| VEGFR-1 | Vascular endothelial growth factor receptor 1 |
| EZH2 | Histone-lysine N-methyltransferase EZH2 |
| EPHA1 | Ephrin type-A receptor 1 |
| AURKA | Aurora kinase A |
| CHEK1 | Serine/threonine-protein kinase Chk1 |
| WEE1 | Wee1-like protein kinase |
| PLK1 | Serine/threonine-protein kinase PLK1 |
| CDC7 | Cell division cycle 7-related protein kinase |
| AURKB | Aurora kinase B |
| PLK4 | Serine/threonine-protein kinase PLK4 |
| MDM2 | E3 ubiquitin-protein ligase Mdm2 |
| MAPK6 | Mitogen-activated protein kinase 6 |
| BUB1 | Mitotic checkpoint serine/threonine-protein kinase BUB1 |
| ESRRA | Steroid hormone receptor ERR1 |
| BCL6 | B-cell lymphoma 6 protein |
| MAPK12 | Mitogen-activated protein kinase 12 |
| CRABP2 | Cellular retinoic acid-binding protein 2 |
| HIPK1 | Homeodomain-interacting protein kinase 1 |
| ADRM1 | Proteasomal ubiquitin receptor ADRM1 |
| CDC20 | Cell division cycle protein 20 homolog |
| BUB1B | Mitotic checkpoint serine/threonine-protein kinase BUB1 beta |
| NTRK2 | BDNF/NT-3 growth factors receptor |
| NTRK1 | High affinity nerve growth factor receptor |
| CD274 | Programmed cell death 1 ligand 1 |
| NUAK1 | NUAK family SNF1-like kinase 1 |
| PDCD1 | Programmed cell death protein 1 |
| ERBB2 | Receptor tyrosine-protein kinase erbB-2 |
| EPHA3 | Ephrin type-A receptor 3 |
| PIK3CG | Phosphatidylinositol 4,5-bisphosphate 3-kinase catalytic subunit gamma |
| | isoform |
| MAP2K1 | Dual specificity mitogen-activated protein kinase kinase 1 |
| LCK | Tyrosine-protein kinase Lck |
| MAP2K2 | Dual specificity mitogen-activated protein kinase kinase 2 |
| JAK3 | Tyrosine-protein kinase JAK3 |
| GSK3B | Glycogen synthase kinase-3 beta |
| JAK2 | Tyrosine-protein kinase JAK2 |
| PRKCI | Protein kinase C iota type |
| FKBP1A | Peptidyl-prolyl cis-trans isomerase FKBP1A |
| DHODH | Dihydroorotate dehydrogenase |
| PIK3CA | Phosphatidylinositol 4,5-bisphosphate 3-kinase catalytic subunit alpha |
| | isoform |
| CDK6 | Cyclin-dependent kinase 6 |
| CDK4 | Cyclin-dependent kinase 4 |
| PDE4B | cAMP-specific 3′,5′-cyclic phosphodiesterase 4B |
| FYN | Tyrosine-protein kinase Fyn |
| TYK2 | Non-receptor tyrosine-protein kinase TYK2 |
| PDK1 | Pyruvate dehydrogenase 1 |
| PDK2 | Pyruvate dehydrogenase 2 |
| PDK3 | Pyruvate dehydrogenase 3 |
| YES1 | Tyrosine-protein kinase Yes |
| GSK3A | Glycogen synthase kinase-3 alpha |
| CDK16 | Cyclin-dependent kinase 16 |
| CSNK2A1 | Casein kinase II subunit alpha |
| CDK7 | Cyclin-dependent kinase 7 |
| PTK2B | Protein-tyrosine kinase 2-beta |
| MAPK10 | Mitogen-activated protein kinase 10 |
| MAPK9 | Mitogen-activated protein kinase 9 |
| MAPK8 | Mitogen-activated protein kinase 8 |
| CDK5 | Cyclin-dependent-like kinase 5 |
| METAP2 | Methionine aminopeptidase 2 |
| BRD2 | Bromodomain-containing protein 2 |
| MAPK3 | Mitogen-activated protein kinase 3 |
| TEC | Tyrosine-protein kinase Tec |
| CDK14 | Cyclin-dependent kinase 14 |
| CDK17 | Cyclin-dependent kinase 17 |
| MAP3K11 | Mitogen-activated protein kinase kinase kinase 11 |
| RPS6KB1 | Ribosomal protein S6 kinase beta-1 |
| CSK | Tyrosine-protein kinase CSK |
| MERTK | Tyrosine-protein kinase Mer |
| STK17B | Serine/threonine-protein kinase 17B |
| CSNK2A2 | Casein kinase II subunit alpha′ |
| RPS6KA1 | Ribosomal protein S6 kinase alpha-1 |
| MAPK13 | Mitogen-activated protein kinase 13 |
| GAK | Cyclin-G-associated kinase |
| CLK1 | Dual specificity protein kinase CLK1 |
| STK4 | Serine/threonine-protein kinase 4 |
| EIF4E | Eukaryotic translation initiation factor 4E |
| STK10 | Serine/threonine-protein kinase 10 |
| LRRK2 | Leucine-rich repeat serine/threonine-protein kinase 2 |
| TAOK3 | Serine/threonine-protein kinase TAO3 |
| MARK2 | Serine/threonine-protein kinase MARK2 |
| CSNK1D | Casein kinase I isoform delta |
| AAK1 | AP2-associated protein kinase 1 |
| IRAK3 | Interleukin-1 receptor-associated kinase 3 |
| STAT3 | Signal transducer and activator of transcription 3 |
| CAMKK1 | Calcium/calmodulin-dependent protein kinase kinase 1 |
| EED | Polycomb protein EED |
| CSNK1A1 | Casein kinase I isoform alpha |
| NEK1 | Serine/threonine-protein kinase Nek1 |
| BMP2K | BMP-2-inducible protein kinase |
| MAPK7 | Mitogen-activated protein kinase 7 |
| ULK1 | Serine/threonine-protein kinase ULK1 |
| RPS6KA3 | Ribosomal protein S6 kinase alpha-3 |
| PTPN11 | Tyrosine-protein phosphatase non-receptor type 11 |
| LIMK2 | LIM domain kinase 2 |
| CSNK1E | Casein kinase I isoform epsilon |
| EIF2AK4 | eIF-2-alpha kinase GCN2 |
| MAP2K5 | Dual specificity mitogen-activated protein kinase kinase 5 |
| MAP4K3 | Mitogen-activated protein kinase kinase kinase kinase 3 |
| VHL | von Hippel-Lindau disease tumor suppressor |
| MARK3 | MAP/microtubule affinity-regulating kinase 3 |
| TAOK2 | Serine/threonine-protein kinase TAO2 |
| MAP4K5 | Mitogen-activated protein kinase kinase kinase kinase 5 |
| SNRK | SNF-related serine/threonine-protein kinase |
| EEF2K | Eukaryotic elongation factor 2 kinase |
| SGK3 | Serine/threonine-protein kinase Sgk3 |
| AHR | Aryl hydrocarbon receptor |
| NEK4 | Serine/threonine-protein kinase Nek4 |
| NEK9 | Serine/threonine-protein kinase Nek9 |
| CIT | Citron Rho-interacting kinase |
| LATS1 | Serine/threonine-protein kinase LATS1 |
| MINK1 | Misshapen-like kinase 1 |
| SIK3 | Serine/threonine-protein kinase SIK3 |
| RPS6KA4 | Ribosomal protein S6 kinase alpha-4 |
| NEK3 | Serine/threonine-protein kinase Nek3 |
| SIK2 | Serine/threonine-protein kinase SIK2 |
| MAST3 | Microtubule-associated serine/threonine-protein kinase 3 |
| STK32C | Serine/threonine-protein kinase 32C |
| ALK | ALK tyrosine kinase receptor |
| EPHB4 | Ephrin type-B receptor 4 |
| PARP2 | Poly [ADP-ribose] polymerase 2 |
| PTK6 | Protein-tyrosine kinase 6 |
| PARP3 | Protein mono-ADP-ribosyltransferase PARP3 |
| CDK2 | Cyclin-dependent kinase 2 |
| CDK8 | Cyclin-dependent kinase 8 |
| BRDT | Bromodomain testis-specific protein |
| MAPKAPK5 | MAP kinase-activated protein kinase 5 |
| MAP3K1 | Mitogen-activated protein kinase kinase kinase 1 |
| CDK18 | Cyclin-dependent kinase 18 |
| CDK10 | Cyclin-dependent kinase 10 |
| TTK | Dual specificity protein kinase TTK |
| PIM2 | Serine/threonine-protein kinase pim-2 |
| PKMYT1 | Membrane-associated tyrosine- and threonine-specific cdc2-inhibitory |
| | kinase |
| MKNK2 | MAP kinase-interacting serine/threonine-protein kinase 2 |
| KAT2B | Histone acetyltransferase KAT2B |
| NEK2 | Serine/threonine-protein kinase Nek2 |
| HASPIN | Serine/threonine-protein kinase haspin |
| PIR | Pirin |
| CYP1B1 | Cytochrome P450 1B1 |
| ERN1 | Serine/threonine-protein kinase/endoribonuclease IRE1 |
| MELK | Maternal embryonic leucine zipper kinase |
| COQ8A | Atypical kinase COQ8A, mitochondrial |
| RIOK2 | Serine/threonine-protein kinase RIO2 |
| RPS6KA6 | Ribosomal protein S6 kinase alpha-6 |
| MAP3K20 | Mitogen-activated protein kinase kinase kinase 20 |
| MAPKAPK3 | MAP kinase-activated protein kinase 3 |
| ULK3 | Serine/threonine-protein kinase ULK3 |
| MAP3K21 | Mitogen-activated protein kinase kinase kinase 21 |
| COQ8B | Atypical kinase COQ8B, mitochondrial |
| TNK1 | Non-receptor tyrosine-protein kinase TNK1 |
| BMPR1A | Bone morphogenetic protein receptor type-1A |
| STK17A | Serine/threonine-protein kinase 17A |
| CDK11A | Cyclin-dependent kinase 11A |
| TESK2 | Dual specificity testis-specific protein kinase 2 |
| NLK | Serine/threonine-protein kinase NLK |
| STK35 | Serine/threonine-protein kinase 35 |
| PKN3 | Serine/threonine-protein kinase N3 |
| STK33 | Serine/threonine-protein kinase 33 |
| SBK1 | Serine/threonine-protein kinase SBK1 |
| SLC9A2 | Sodium/hydrogen exchanger 2 |
| CSNK2A3 | Casein kinase II subunit alpha 3 |
| TACC3 | Transforming acidic coiled-coil-containing protein 3 |
| GSPT1 | Eukaryotic peptide chain release factor GTP-binding subunit ERF3A |
| SLC9A7 | Sodium/hydrogen exchanger 7 |
| RPS6KC1 | Ribosomal protein S6 kinase delta-1 |
| TBCK | TBC domain-containing protein kinase-like protein |
| CRABP1 | Cellular retinoic acid-binding protein 1 |
| BCKDK | [3-methyl-2-oxobutanoate dehydrogenase [lipoamide]] kinase, |
| | mitochondrial |
| TRPM7 | Transient receptor potential cation channel subfamily M member 7 |
| TRIB3 | Tribbles homolog 3 |
| UHMK1 | Serine/threonine-protein kinase Kist |
| PDIK1L | Serine/threonine-protein kinase PDIK1L |
| STK40 | Serine/threonine-protein kinase 40 |
| SLC9A4 | Sodium/hydrogen exchanger 4 |
| EPHB3 | Ephrin type-B receptor 3 |
| NTRK3 | NT-3 growth factor receptor |
| MAP3K12 | Mitogen-activated protein kinase kinase kinase 12 |
| MAPK11 | Mitogen-activated protein kinase 11 |
| DDR2 | Discoidin domain-containing receptor 2 |
| RARB | Retinoic acid receptor beta |
| PDE4C | cAMP-specific 3′,5′-cyclic phosphodiesterase 4C |
| IDO1 | Indoleamine 2,3-dioxygenase 1 |
| SLC9B1 | Sodium/hydrogen exchanger 9B1 |
| PRAG1 | Inactive tyrosine-protein kinase PRAG1 |
| TOP1 | DNA topoisomerase 1 |
| TOP2A | DNA topoisomerase 2-alpha |
| CXCR4 | C-X-C chemokine receptor type 4 |
| ERBB4 | Receptor tyrosine-protein kinase erbB-4 |
| HCK | Tyrosine-protein kinase HCK |
| SYK | Tyrosine-protein kinase SYK |
| ACLY | ATP-citrate synthase |
| IMPDH2 | Inosine-5′-monophosphate dehydrogenase 2 |
| CYP3A4 | Cytochrome P450 3A4 |
| TOP2B | DNA topoisomerase 2-beta |
| KEAP1 | Kelch-like ECH-associated protein 1 |
| NR3C1 | Glucocorticoid receptor |
| DNMT1 | DNA (cytosine-5)-methyltransferase 1 |
| TXNRD1 | Thioredoxin reductase 1, cytoplasmic |
| HDAC4 | Histone deacetylase 4 |
| PDGFRA | Platelet-derived growth factor receptor alpha |
| TUBB | Tubulin beta chain |
| TUBB2B | Tubulin beta-2B chain |
| GABRA5 | Gamma-aminobutyric acid receptor subunit alpha-5 |
| EPHB1 | Ephrin type-B receptor 1 |
| KCNQ2 | Potassium voltage-gated channel subfamily KQT member 2 |
| GRIK2 | Glutamate receptor ionotropic, kainate 2 |
| CALM1 | Calmodulin-1 |
| HDAC5 | Histone deacetylase 5 |
| DNMT3A | DNA (cytosine-5)-methyltransferase 3A |
| KIF5B | Kinesin-1 heavy chain |
| POLD1 | DNA polymerase delta catalytic subunit |
| CUL4A | Cullin-4A |
| TUBB2A | Tubulin beta-2A chain |
| PDGFRB | Platelet-derived growth factor receptor beta |
| FGR | Tyrosine-protein kinase Fgr |
| PRKCG | Protein kinase C gamma type |
| SCN3A | Sodium channel protein type 3 subunit alpha |
| HDAC9 | Histone deacetylase 9 |
| BACE1 | Beta-secretase 1 |
| HSP90AA1 | Heat shock protein HSP 90-alpha |
| HSP90AB1 | Heat shock protein HSP 90-beta |
| PDPK1 | 3-phosphoinositide-dependent protein kinase 1 |
| BIRC2 | Baculoviral IAP repeat-containing protein 2 |
| CHEK2 | Serine/threonine-protein kinase Chk2 |
| MAP3K5 | Mitogen-activated protein kinase kinase kinase 5 |
| CASP1 | Caspase-1 |
| NAE1 | NEDD8-activating enzyme E1 regulatory subunit |
| PIK3R1 | Phosphatidylinositol 3-kinase regulatory subunit alpha |
| IKBKB | Inhibitor of nuclear factor kappa-B kinase subunit beta |
| PSMA1 | Proteasome subunit alpha type-1 |
| ITGB1 | Integrin beta-1 |
| PRKDC | DNA-dependent protein kinase catalytic subunit |
| PIK3R2 | Phosphatidylinositol 3-kinase regulatory subunit beta |
| FGF2 | Fibroblast growth factor 2 |
| ZAP70 | Tyrosine-protein kinase ZAP-70 |
| PIP4K2B | Phosphatidylinositol 5-phosphate 4-kinase type-2 beta |
| GAPDH | Glyceraldehyde-3-phosphate dehydrogenase |
| FASN | Fatty acid synthase |
| PKM | Pyruvate kinase PKM |
| VCP | Transitional endoplasmic reticulum ATPase |
| KDM1A | Lysine-specific histone demethylase 1A |
| TAB1 | TGF-beta-activated kinase 1 and MAP3K7-binding protein 1 |
| EP300 | Histone acetyltransferase p300 |
| VRK1 | Serine/threonine-protein kinase VRK1 |
| RPS6KA5 | Ribosomal protein S6 kinase alpha-5 |
| UBA2 | SUMO-activating enzyme subunit 2 |
| LDHA | L-lactate dehydrogenase A chain |
| TKT | Transketolase |
| HPRT1 | Hypoxanthine-guanine phosphoribosyltransferase |
| KDM2A | Lysine-specific demethylase 2A |
| EHMT1 | Histone-lysine N-methyltransferase EHMT1 |
| CAMK1D | Calcium/calmodulin-dependent protein kinase type 1D |
| WDR5 | WD repeat-containing protein 5 |
| EHMT2 | Histone-lysine N-methyltransferase EHMT2 |
| RAC1 | Ras-related C3 botulinum toxin substrate 1 |
| OGT | UDP-N-acetylglucosamine--peptide N-acetylglucosaminyltransferase |
| | 110 kDa subunit |
| NCOA1 | Nuclear receptor coactivator 1 |
| PHGDH | D-3-phosphoglycerate dehydrogenase |
| ARHGDIA | Rho GDP-dissociation inhibitor 1 |
| RBBP4 | Histone-binding protein RBBP4 |
| BPTF | Nucleosome-remodeling factor subunit BPTF |
| KAT5 | Histone acetyltransferase KAT5 |
| NOTCH1 | Neurogenic locus notch homolog protein 1 |
| PPIA | Peptidyl-prolyl cis-trans isomerase A |
| FKBP4 | Peptidyl-prolyl cis-trans isomerase FKBP4 |
| CREBBP | CREB-binding protein |
| POLB | DNA polymerase beta |
| APEX1 | DNA-(apurinic or apyrimidinic site) endonuclease |
| CCNT1 | Cyclin-T1 |
| EIF4A1 | Eukaryotic initiation factor 4A-I |
| USP7 | Ubiquitin carboxyl-terminal hydrolase 7 |
| CTNNB1 | Catenin beta-1 |
| TXN | Thioredoxin |
| DDX3X | ATP-dependent RNA helicase DDX3X |
| NLRP3 | NACHT, LRR and PYD domains-containing protein 3 |
| TNKS | Poly [ADP-ribose] polymerase tankyrase-1 |
| PAK3 | Serine/threonine-protein kinase PAK 3 |
| PKN1 | Serine/threonine-protein kinase N1 |
| CISD1 | CDGSH iron-sulfur domain-containing protein 1 |
| WNK1 | Serine/threonine-protein kinase WNK1 |
| BRD1 | Bromodomain-containing protein 1 |
| XIAP | E3 ubiquitin-protein ligase XIAP |
| PRDX1 | Peroxiredoxin-1 |
| KMT2A | Histone-lysine N-methyltransferase 2A |
| KDM5C | Lysine-specific demethylase 5C |
| RPA1 | Replication protein A 70 kDa DNA-binding subunit |
| CAMK1 | Calcium/calmodulin-dependent protein kinase type 1 |
| EIF4A3 | Eukaryotic initiation factor 4A-III |
| TAF1 | Transcription initiation factor TFIID subunit 1 |
| ALKBH3 | Alpha-ketoglutarate-dependent dioxygenase alkB homolog 3 |
| MAP3K2 | Mitogen-activated protein kinase kinase kinase 2 |
| RELA | Transcription factor p65 |
| NCOR2 | Nuclear receptor corepressor 2 |
| PRPF4B | Serine/threonine-protein kinase PRP4 homolog |
| RPS3 | 40S ribosomal protein S3 |
| RPSA | 40S ribosomal protein SA |
| RPS27A | Ubiquitin-40S ribosomal protein S27a |
| RPS20 | 40S ribosomal protein S20 |
| RPL18 | 60S ribosomal protein L18 |
| RPS5 | 40S ribosomal protein S5 |
| RPL6 | 60S ribosomal protein L6 |
| RPLP0 | 60S acidic ribosomal protein P0 |
| RPL3 | 60S ribosomal protein L3 |
| RPL18A | 60S ribosomal protein L18a |
| RPL28 | 60S ribosomal protein L28 |
| RPL19 | 60S ribosomal protein L19 |
| RPL34 | 60S ribosomal protein L34 |
| RPS13 | 40S ribosomal protein S13 |
| RPL24 | 60S ribosomal protein L24 |
| RPS15 | 40S ribosomal protein S15 |
| RPL5 | 60S ribosomal protein L5 |
| RPS10 | 40S ribosomal protein S10 |
| RPL35 | 60S ribosomal protein L35 |
| RPS6 | 40S ribosomal protein S6 |
| RPS24 | 40S ribosomal protein S24 |
| RPS2 | 40S ribosomal protein S2 |
| RPL11 | 60S ribosomal protein L11 |
| RPS8 | 40S ribosomal protein S8 |
| RPL32 | 60S ribosomal protein L32 |
| RPS3A | 40S ribosomal protein S3a |
| RPL10 | 60S ribosomal protein L10 |
| RPL7A | 60S ribosomal protein L7a |
| RPL30 | 60S ribosomal protein L30 |
| RPL8 | 60S ribosomal protein L8 |
| RPL26 | 60S ribosomal protein L26 |
| RPS14 | 40S ribosomal protein S14 |
| RPL13 | 60S ribosomal protein L13 |
| RPS9 | 40S ribosomal protein S9 |
| RPS21 | 40S ribosomal protein S21 |
| RPS7 | 40S ribosomal protein S7 |
| RPL38 | 60S ribosomal protein L38 |
| RPL4 | 60S ribosomal protein L4 |
| RPL15 | 60S ribosomal protein L15 |
| RPS17 | 40S ribosomal protein S17 |
| RPS23 | 40S ribosomal protein S23 |
| RPL14 | 60S ribosomal protein L14 |
| RPL12 | 60S ribosomal protein L12 |
| RPS4X | 40S ribosomal protein S4, X isoform |
| RPL23A | 60S ribosomal protein L23a |
| RPL10A | 60S ribosomal protein L23a |
| RPS18 | 40S ribosomal protein S18 |
| SLC7A5 | Large neutral amino acids transporter small subunit 1 |
| ORAI1 | Calcium release-activated calcium channel protein 1 |
| PI4K2A | Phosphatidylinositol 4-kinase type 2-alpha |
| SUMO1 | Small ubiquitin-related modifier 1 |
| HSPA8 | Heat shock cognate 71 kDa protein |
| HSPD1 | 60 kDa heat shock protein, mitochondrial |
| CSNK2B | Casein kinase II subunit beta |
| PPP2CA | Serine/threonine-protein phosphatase 2A catalytic subunit alpha isoform |
| FSCN1 | Fascin |
| BID | BH3-interacting domain death agonist |
| DCUN1D1 | DCN1-like protein 1 |
| USP28 | Ubiquitin carboxyl-terminal hydrolase 28 |
| TP53BP1 | TP53-binding protein 1 |
| KDM5A | Lysine-specific demethylase 5A |
| BAZ2A | Bromodomain adjacent to zinc finger domain protein 2A |
| UBE2I | SUMO-conjugating enzyme UBC9 |
| SPIN1 | Spindlin-1 |
| TDP2 | Tyrosyl-DNA phosphodiesterase 2 |
| KDM5B | Lysine-specific demethylase 5B |
| RBBP5 | Retinoblastoma-binding protein 5 |
| PRMT1 | Protein arginine N-methyltransferase 1 |
| MLLT1 | Protein ENL |
| SETDB1 | Histone-lysine N-methyltransferase SETDB1 |
| ARNT | Aryl hydrocarbon receptor nuclear translocator |
| SNRNP200 | U5 small nuclear ribonucleoprotein 200 kDa helicase |
| ATAD2 | ATPase family AAA domain-containing protein 2 |
| SQSTM1 | Sequestosome-1 |
| DPY30 | Protein dpy-30 homolog |
| WRN | Werner syndrome ATP-dependent helicase |
| JMJD1C | Probable JmjC domain-containing histone demethylation protein 2C |
| MDM4 | Protein Mdm4 |
| L3MBTL3 | Lethal(3)malignant brain tumor-like protein 3 |
| LDLR | Low-density lipoprotein receptor |
| GLUL | Glutamine synthetase |
| HRAS | GTPase HRas |
| APOBEC3G | DNA dC−>dU-editing enzyme APOBEC-3G |
| TSG101 | Tumor susceptibility gene 101 protein |
| LRP6 | Low-density lipoprotein receptor-related protein 6 |
| ENO1 | Alpha-enolase |
| ANXA2 | Annexin A2 |
| SOD1 | Superoxide dismutase [Cu—Zn] |
| KAT6A | Histone acetyltransferase KAT6A |
| CBS | Cystathionine beta-synthase |
| RUVBL1 | RuvB-like 1 |
| UHRF1 | E3 ubiquitin-protein ligase UHRF1 |
| HSPA5 | Endoplasmic reticulum chaperone BiP |
| SQLE | Squalene monooxygenase |
| NEDD4 | E3 ubiquitin-protein ligase NEDD4 |
| RBBP7 | Histone-binding protein RBBP7 |
| UBE2N | Ubiquitin-conjugating enzyme E2 N |
| NRAS | GTPase NRas |
| PTEN | Phosphatidylinositol 3,4,5-trisphosphate 3-phosphatase and dual-specificity |
| | protein phosphatase PTEN |
| RHOA | Transforming protein RhoA |
| RNF31 | E3 ubiquitin-protein ligase RNF31 |
| PIN4 | Peptidyl-prolyl cis-trans isomerase NIMA-interacting 4 |
| TPI1 | Triosephosphate isomerase |
| PPIG | Peptidyl-prolyl cis-trans isomerase G |
| NCOA2 | Nuclear receptor coactivator 2 |
| FOXO1 | Forkhead box protein O1 |
| PSIP1 | PC4 and SFRS1-interacting protein |
| MACROD2 | ADP-ribose glycohydrolase MACROD2 |
| SETD2 | Histone-lysine N-methyltransferase SETD2 |
| NFKBIA | NF-kappa-B inhibitor alpha |
| PLA2G4A | Cytosolic phospholipase A2 |
| ICAM1 | Intercellular adhesion molecule 1 |
| ACVR1B | Activin receptor type-1B |
| CYBB | Cytochrome b-245 heavy chain |
| STAMBP | STAM-binding protein |
| NFKB1 | Nuclear factor NF-kappa-B p105 subunit |
| PAK2 | Serine/threonine-protein kinase PAK 2 |
| ITPR1 | Inositol 1,4,5-trisphosphate receptor type 1 |
| ELAVL1 | ELAV-like protein 1 |
| NFKB2 | Nuclear factor NF-kappa-B p100 subunit |
| STAT1 | Signal transducer and activator of transcription 1-alpha/beta |
| STAT5A | Signal transducer and activator of transcription 5A |
| RIPK3 | Receptor-interacting serine/threonine-protein kinase 3 |
| BRSK2 | Serine/threonine-protein kinase BRSK2 |
| ERCC4 | DNA repair endonuclease XPF |
| NPM1 | Nucleophosmin |
| IKBKG | NF-kappa-B essential modulator |
| PBK | Lymphokine-activated killer T-cell-originated protein kinase |
| CD22 | B-cell receptor CD22 |
| APC | Adenomatous polyposis coli protein |
| PLCG1 | 1-phosphatidylinositol 4,5-bisphosphate phosphodiesterase gamma-1 |
| ING2 | Inhibitor of growth protein 2 |
| ERCC1 | DNA excision repair protein ERCC-1 |
| KDM2B | Lysine-specific demethylase 2B |
| ETV6 | Transcription factor ETV6 |
| CBX4 | E3 SUMO-protein ligase CBX4 |
| USP25 | Ubiquitin carboxyl-terminal hydrolase 25 |
| HSF1 | Heat shock factor protein 1 |
| TRIM33 | E3 ubiquitin-protein ligase TRIM33 |
| HNRNPA1 | Heterogeneous nuclear ribonucleoprotein A1 |
| TERF2 | Telomeric repeat-binding factor 2 |
| PTBP1 | Polypyrimidine tract-binding protein 1 |
| NR2C2 | Nuclear receptor subfamily 2 group C member 2 |
| NOTCH2 | Neurogenic locus notch homolog protein 2 |
| USP8 | Ubiquitin carboxyl-terminal hydrolase 8 |
| RGS12 | Regulator of G-protein signaling 12 |
| ATF1 | Cyclic AMP-dependent transcription factor ATF-1 |
| PPM1B | Protein phosphatase 1B |
| PDCD4 | Programmed cell death protein 4 |
| LMNA | Prelamin-A/C |
| MITF | Microphthalmia-associated transcription factor |
| ADCY3 | Adenylate cyclase type 3 |
| EEF1A1 | Elongation factor 1-alpha 1 |
| MYH9 | Myosin-9 |
| BCL3 | B-cell lymphoma 3 protein |
| XRCC5 | X-ray repair cross-complementing protein 5 |
| USP4 | Ubiquitin carboxyl-terminal hydrolase 4 |
| AGL | Glycogen debranching enzyme |
| XRCC6 | X-ray repair cross-complementing protein 6 |
| MAGI3 | Membrane-associated guanylate kinase, WW and PDZ domain-containing |
| | protein 3 |
| DVL1 | Segment polarity protein dishevelled homolog DVL-1 |
| VDAC2 | Voltage-dependent anion-selective channel protein 2 |
| MDH1 | Malate dehydrogenase, cytoplasmic |
| CBX5 | Chromobox protein homolog 5 |
| USP10 | Ubiquitin carboxyl-terminal hydrolase 10 |
| CBX1 | Chromobox protein homolog 1 |
| BRD8 | Bromodomain-containing protein 8 |
| TSNAX | Translin-associated protein X |
| CREB1 | Cyclic AMP-responsive element-binding protein 1 |
| RBCK1 | RanBP-type and C3HC4-type zinc finger-containing protein 1 |
| UBA1 | Ubiquitin-like modifier-activating enzyme 1 |
| MACROD1 | ADP-ribose glycohydrolase MACROD1 |
| PELP1 | Proline-, glutamic acid- and leucine-rich protein 1 |
| BAP1 | Ubiquitin carboxyl-terminal hydrolase BAP1 |
| TERF2IP | Telomeric repeat-binding factor 2-interacting protein 1 |
| LARP7 | La-related protein 7 |
| UBA7 | Ubiquitin-like modifier-activating enzyme 7 |
| SUMO3 | Small ubiquitin-related modifier 3 |
| SUMO2 | Small ubiquitin-related modifier 2 |
| GIGYF2 | GRB10-interacting GYF protein 2 |
| S100A10 | Protein S100-A10 |
| GRIK3 | Glutamate receptor ionotropic, kainate 3 |
| TLR8 | Toll-like receptor 8 |
| DYRK2 | Dual specificity tyrosine-phosphorylation-regulated kinase 2 |
| NOS1 | Nitric oxide synthase, brain |
| WNK3 | Serine/threonine-protein kinase WNK3 |
| TLR2 | Toll-like receptor 2 |
| BCL2L11 | Bcl-2-like protein 11 |
| SLC11A2 | Natural resistance-associated macrophage protein 2 |
| ABCB11 | Bile salt export pump |
| ITGAL | Integrin alpha-L |
| ROCK1 | Rho-associated protein kinase 1 |
| CA12 | Carbonic anhydrase 12 |
| EPHA7 | Ephrin type-A receptor 7 |
| PRKCA | Protein kinase C alpha type |
| CA2 | Carbonic anhydrase 2 |
| PDE2A | cGMP-dependent 3′,5′-cyclic phosphodiesterase |
| EPHA4 | Ephrin type-A receptor 4 |
| ACE | Angiotensin-converting enzyme |
| RAF1 | RAF proto-oncogene serine/threonine-protein kinase |
| GLA | Alpha-galactosidase A |
| MAOB | Amine oxidase [flavin-containing] B |
| AKR1B1 | Aldo-keto reductase family 1 member B1 |
| GSR | Glutathione reductase, mitochondrial |
| UMPS | Uridine 5′-monophosphate synthase |
| THRA | Thyroid hormone receptor alpha |
| GART | Trifunctional purine biosynthetic protein adenosine-3 [Includes: |
| | Phosphoribosylamine--glycine ligase |
| FDPS | Farnesyl pyrophosphate synthase |
| ADH5 | Alcohol dehydrogenase class-3 |
| RXRB | Retinoic acid receptor RXR-beta |
| DHFR | Dihydrofolate reductase |
| PDE10A | cAMP and cAMP-inhibited cGMP 3′,5′-cyclic phosphodiesterase 10A |
| IDH1 | Isocitrate dehydrogenase [NADP] cytoplasmic |
| ITGB2 | Integrin beta-2 |
| COMT | Catechol O-methyltransferase |
| PRKCQ | Protein kinase C theta type |
| IMPDH1 | Inosine-5′-monophosphate dehydrogenase 1 |
| HDAC7 | Histone deacetylase 7 |
| TUBB3 | Tubulin beta-3 chain |
| TUBA1A | Tubulin alpha-1A chain |
| ADA | Adenosine deaminase |
| GABRG2 | Gamma-aminobutyric acid receptor subunit gamma-2 |
| GABBR2 | Gamma-aminobutyric acid type B receptor subunit 2 |
| GABRB2 | Gamma-aminobutyric acid receptor subunit beta-2 |
| GABRB3 | Gamma-aminobutyric acid receptor subunit beta-3 |
| GABBR1 | Gamma-aminobutyric acid type B receptor subunit 1 |
| PRKCE | Protein kinase C epsilon type |
| SIGMAR1 | Sigma non-opioid intracellular receptor 1 |
| MTOR | Serine/threonine-protein kinase mTOR |
| SLC29A1 | Equilibrative nucleoside transporter 1 |
| KCNJ11 | ATP-sensitive inward rectifier potassium channel 11 |
| GAA | Lysosomal alpha-glucosidase |
| PRKCH | Protein kinase C eta type |
| PIK3CD | Phosphatidylinositol 4,5-bisphosphate 3-kinase catalytic subunit delta |
| | isoform |
| ROCK2 | Rho-associated protein kinase 2 |
| MAOA | Amine oxidase [flavin-containing] A |
| SOAT1 | Sterol O-acyltransferase 1 |
| BCR | Breakpoint cluster region protein |
| PSMB5 | Proteasome subunit beta type-5 |
| XPO1 | Exportin-1 |
| MALT1 | Mucosa-associated lymphoid tissue lymphoma translocation protein 1 |
| IDH2 | Isocitrate dehydrogenase [NADP], mitochondrial |
| RRM1 | Ribonucleoside-diphosphate reductase large subunit |
| NDUFA10 | NADH dehydrogenase [ubiquinone] 1 alpha subcomplex subunit 10, |
| | mitochondrial |
| EPHA5 | Ephrin type-A receptor 5 |
| TUBB4B | Tubulin beta-4B chain |
| TUBB1 | Tubulin beta-1 chain |
| TUBB4A | Tubulin beta-4A chain |
| TUBB6 | Tubulin beta-6 chain |
| TUBA4A | Tubulin alpha-4A chain |
| TUBA1C | Tubulin alpha-1C chain |
| CACNB1 | Voltage-dependent L-type calcium channel subunit beta-1 |
| ATP1A2 | Sodium/potassium-transporting ATPase subunit alpha-2 |
| ATP1A3 | Sodium/potassium-transporting ATPase subunit alpha-3 |
| GRIA1 | Glutamate receptor 1 |
| GABRB1 | Gamma-aminobutyric acid receptor subunit beta-1 |
| ATP1A1 | Sodium/potassium-transporting ATPase subunit alpha-1 |
| KCNQ5 | Potassium voltage-gated channel subfamily KQT member 5 |
| PRKCD | Protein kinase C delta type |
| CACNA1H | Voltage-dependent T-type calcium channel subunit alpha-1H |
| GRIA4 | Glutamate receptor 4 |
| KCNA3 | Potassium voltage-gated channel subfamily A member 3 |
| S1PR3 | Sphingosine 1-phosphate receptor 3 |
| NISCH | Nischarin |
| PRKCB | Protein kinase C beta type |
| TLR7 | Toll-like receptor 7 |
| CACNA1B | Voltage-dependent N-type calcium channel subunit alpha-1B |
| CACNA2D2 | Voltage-dependent calcium channel subunit alpha-2/delta-2 |
| CPT2 | Carnitine O-palmitoyltransferase 2, mitochondrial |
| FAAH | Fatty-acid amide hydrolase 1 |
| UGCG | Ceramide glucosyltransferase |
| PDE1A | Calcium/calmodulin-dependent 3′,5′-cyclic nucleotide phosphodiesterase 1A |
| HPD | 4-hydroxyphenylpyruvate dioxygenase |
| CA5B | Carbonic anhydrase 5B, mitochondrial |
| CA5A | Carbonic anhydrase 5A, mitochondrial |
| ANO1 | Anoctamin-1 |
| DHCR24 | Delta(24)-sterol reductase |
| CCDC6 | Coiled-coil domain-containing protein 6 |
| EML4 | Echinoderm microtubule-associated protein-like 4 |
| KCNA2 | Potassium voltage-gated channel subfamily A member 2 |
| KCNB2 | Potassium voltage-gated channel subfamily B member 2 |
| PLA2G7 | Platelet-activating factor acetylhydrolase |
| GRM5 | Metabotropic glutamate receptor 5 |
| PLA2G2A | Phospholipase A2, membrane associated |
| MIF | Macrophage migration inhibitory factor |
| GBA | Lysosomal acid glucosylceramidase |
| INSR | Insulin receptor |
| GSTP1 | Glutathione S-transferase P |
| PNP | Purine nucleoside phosphorylase |
| FDFT1 | Squalene synthase |
| CASP8 | Caspase-8 |
| CASP9 | Caspase-9 |
| CASP6 | Caspase-6 |
| CASP3 | Caspase-3 |
| CASP7 | Caspase-7 |
| ERAP1 | Endoplasmic reticulum aminopeptidase 1 |
| UBA3 | NEDD8-activating enzyme E1 catalytic subunit |
| PSEN1 | Presenilin-1 |
| FNTA | Protein farnesyltransferase/geranylgeranyltransferase type-1 subunit alpha |
| METAP1 | Methionine aminopeptidase 1 |
| CASP2 | Caspase-2 |
| ITGA4 | Integrin alpha-4 |
| ABCB1 | ATP-dependent translocase ABCB1 |
| NTSR1 | Neurotensin receptor type 1 |
| GRM3 | Metabotropic glutamate receptor 3 |
| LNPEP | Leucyl-cystinyl aminopeptidase |
| APH1A | Gamma-secretase subunit APH-1A |
| NCSTN | Nicastrin |
| PSMB1 | Proteasome subunit beta type-1 |
| PSMA2 | Proteasome subunit alpha type-2 |
| PSMB7 | Proteasome subunit beta type-7 |
| PSMA5 | Proteasome subunit alpha type-5 |
| PSMA4 | Proteasome subunit alpha type-4 |
| PSMA3 | Proteasome subunit alpha type-3 |
| PSMA6 | Proteasome subunit alpha type-6 |
| PSMA7 | Proteasome subunit alpha type-7 |
| PSMB2 | Proteasome subunit beta type-2 |
| EGLN1 | Egl nine homolog 1 |
| PSMB6 | Proteasome subunit beta type-6 |
| PSMB4 | Proteasome subunit beta type-4 |
| PSMB8 | Proteasome subunit beta type-8 |
| PSMB10 | Proteasome subunit beta type-10 |
| PSMB9 | Proteasome subunit beta type-9 |
| FNTB | Protein farnesyltransferase subunit beta |
| PSMB3 | Proteasome subunit beta type-3 |
| ERAP2 | Endoplasmic reticulum aminopeptidase 2 |
| DNPEP | Aspartyl aminopeptidase |
| STS | Steryl-sulfatase |
| SELL | L-selectin |
| CCR1 | C-C chemokine receptor type 1 |
| PIK3CB | Phosphatidylinositol 4,5-bisphosphate 3-kinase catalytic subunit beta |
| | isoform |
| PORCN | Protein-serine O-palmitoleoyltransferase porcupine |
| P4HTM | Transmembrane prolyl 4-hydroxylase |
| ATR | Serine/threonine-protein kinase ATR |
| CDK19 | Cyclin-dependent kinase 19 |
| CDK3 | Cyclin-dependent kinase 3 |
| CASP4 | Caspase-4 |
| PIK3R5 | Phosphoinositide 3-kinase regulatory subunit 5 |
| CASP10 | Caspase-10 |
| NPEPPS | Puromycin-sensitive aminopeptidase |
| RNPEP | Aminopeptidase B |
| LAP3 | Cytosol aminopeptidase |
| CDK20 | Cyclin-dependent kinase 20 |
| DPYD | Dihydropyrimidine dehydrogenase [NADP(+)] |
| XPNPEP1 | Xaa-Pro aminopeptidase 1 |
| NAMPT | Nicotinamide phosphoribosyltransferase |
| PRKACA | cAMP-dependent protein kinase catalytic subunit alpha |
| PSMD14 | 26S proteasome non-ATPase regulatory subunit 14 |
| ARAF | Serine/threonine-protein kinase A-Raf |
| PRKACB | cAMP-dependent protein kinase catalytic subunit beta |
| ITGAV | Integrin alpha-V |
| TGM2 | Protein-glutamine gamma-glutamyltransferase 2 |
| ACE2 | Angiotensin-converting enzyme 2 |
| PAK1 | Serine/threonine-protein kinase PAK 1 |
| CAPN1 | Calpain-1 catalytic subunit |
| CDC42 | Cell division control protein 42 homolog |
| NMT1 | Glycylpeptide N-tetradecanoyltransferase 1 |
| CAMK2D | Calcium/calmodulin-dependent protein kinase type II subunit delta |
| AKR1B10 | Aldo-keto reductase family 1 member B10 |
| P4HB | Protein disulfide-isomerase |
| CDC42BPB | Serine/threonine-protein kinase MRCK beta |
| CAMK2G | Calcium/calmodulin-dependent protein kinase type II subunit gamma |
| PTP4A1 | Protein tyrosine phosphatase type IVA 1 |
| G6PD | Glucose-6-phosphate 1-dehydrogenase |
| LSS | Lanosterol synthase |
| ALB | Albumin |
| ECE1 | Endothelin-converting enzyme 1 |
| CTSD | Cathepsin D |
| CTSB | Cathepsin B |
| ILK | Integrin-linked protein kinase |
| MGLL | Monoglyceride lipase |
| CASK | Peripheral plasma membrane protein CASK |
| ADAM17 | Disintegrin and metalloproteinase domain-containing protein 17 |
| PYGL | Glycogen phosphorylase, liver form |
| ATIC | Bifunctional purine biosynthesis protein ATIC |
| HSD17B10 | 3-hydroxyacyl-CoA dehydrogenase type-2 |
| SRPK1 | SRSF protein kinase 1 |
| LTA4H | Leukotriene A-4 hydrolase |
| STK16 | Serine/threonine-protein kinase 16 |
| ADK | Adenosine kinase |
| GRK6 | G protein-coupled receptor kinase 6 |
| ACVR1 | Activin receptor type-1 |
| CES1 | Liver carboxylesterase 1 |
| CSNK1G3 | Casein kinase I isoform gamma-3 |
| CYP51A1 | Lanosterol 14-alpha demethylase |
| CAMK2A | Calcium/calmodulin-dependent protein kinase type II subunit alpha |
| BCAT1 | Branched-chain-amino-acid aminotransferase, cytosolic |
| MAP2K4 | Dual specificity mitogen-activated protein kinase kinase 4 |
| ACACB | Acetyl-CoA carboxylase 2 |
| MTAP | S-methyl-5′-thioadenosine phosphorylase |
| AHCY | Adenosylhomocysteinase |
| STK24 | Serine/threonine-protein kinase 24 |
| GALK1 | Galactokinase |
| MAP2K6 | Dual specificity mitogen-activated protein kinase kinase 6 |
| DCPS | m7GpppX diphosphatase |
| LDHB | L-lactate dehydrogenase B chain |
| EPHX2 | Bifunctional epoxide hydrolase 2 [Includes: Cytosolic epoxide hydrolase 2 |
| NQO2 | Ribosyldihydronicotinamide dehydrogenase [quinone] |
| SRPK2 | SRSF protein kinase 2 |
| SAE1 | SUMO-activating enzyme subunit 1 |
| CARM1 | Histone-arginine methyltransferase CARM1 |
| CAMK4 | Calcium/calmodulin-dependent protein kinase type IV |
| TNIK | TRAF2 and NCK-interacting protein kinase |
| DCK | Deoxycytidine kinase |
| PHKG2 | Phosphorylase b kinase gamma catalytic chain, liver/testis isoform |
| DYRK1A | Dual specificity tyrosine-phosphorylation-regulated kinase 1A |
| CBR1 | Carbonyl reductase [NADPH] 1 |
| DAPK3 | Death-associated protein kinase 3 |
| FEN1 | Flap endonuclease 1 |
| OXSR1 | Serine/threonine-protein kinase OSR1 |
| CLK3 | Dual specificity protein kinase CLK3 |
| UPP1 | Uridine phosphorylase 1 |
| AKR1C1 | Aldo-keto reductase family 1 member C1 |
| AKR1C3 | Aldo-keto reductase family 1 member C3 |
| CD38 | ADP-ribosyl cyclase/cyclic ADP-ribose hydrolase 1 |
| SORT1 | Sortilin |
| GALNT2 | Polypeptide N-acetylgalactosaminyltransferase 2 |
| LGALS1 | Galectin-1 |
| HK2 | Hexokinase-2 |
| HK1 | Hexokinase-1 |
| HMOX1 | Heme oxygenase 1 |
| PARP14 | Protein mono-ADP-ribosyltransferase PARP14 |
| GRB2 | Growth factor receptor-bound protein 2 |
| HMOX2 | Heme oxygenase 2 |
| DDAH1 | N(G),N(G)-dimethylarginine dimethylaminohydrolase 1 |
| PARP12 | Protein mono-ADP-ribosyltransferase PARP12 |
| OAT | Ornithine aminotransferase, mitochondrial |
| SIRT6 | NAD-dependent protein deacetylase sirtuin-6 |
| PGK1 | Phosphoglycerate kinase 1 |
| KAT8 | Histone acetyltransferase KAT8 |
| SPR | Sepiapterin reductase |
| CTH | Cystathionine gamma-lyase |
| SIRT5 | NAD-dependent protein deacylase sirtuin-5, mitochondrial |
| GSTO1 | Glutathione S-transferase omega-1 |
| PFKFB3 | 6-phosphofructo-2-kinase/fructose-2,6-bisphosphatase 3 |
| CTBP2 | C-terminal-binding protein 2 |
| PARP10 | Protein mono-ADP-ribosyltransferase PARP10 |
| SMYD3 | Histone-lysine N-methyltransferase SMYD3 |
| SULT1A1 | Sulfotransferase 1A1 |
| AKR1C4 | Aldo-keto reductase family 1 member C4 |
| GSTM2 | Glutathione S-transferase Mu 2 |
| STRADA | STE20-related kinase adapter protein alpha |
| BMPR2 | Bone morphogenetic protein receptor type-2 |
| PIK3C2A | Phosphatidylinositol 4-phosphate 3-kinase C2 domain-containing subunit |
| | alpha |
| PTPN1 | Tyrosine-protein phosphatase non-receptor type 1 |
| IDE | Insulin-degrading enzyme |
| CAPNS1 | Calpain small subunit 1 |
| GLO1 | Lactoylglutathione lyase |
| UCHL1 | Ubiquitin carboxyl-terminal hydrolase isozyme L1 |
| NPC1 | NPC intracellular cholesterol transporter 1 |
| FES | Tyrosine-protein kinase Fes/Fps |
| CSNK1G1 | Casein kinase I isoform gamma-1 |
| SLK | STE20-like serine/threonine-protein kinase |
| MAP2K7 | Dual specificity mitogen-activated protein kinase kinase 7 |
| PIK3C3 | Phosphatidylinositol 3-kinase catalytic subunit type 3 |
| HAO1 | Hydroxyacid oxidase 1 |
| PASK | PAS domain-containing serine/threonine-protein kinase |
| STK25 | Serine/threonine-protein kinase 25 |
| KYNU | Kynureninase |
| DUT | Deoxyuridine 5′-triphosphate nucleotidohydrolase, mitochondrial |
| PTPN22 | Tyrosine-protein phosphatase non-receptor type 22 |
| NEK7 | Serine/threonine-protein kinase Nek7 |
| NNMT | Nicotinamide N-methyltransferase |
| NRP1 | Neuropilin-1 |
| RPS27 | 40S ribosomal protein S27 |
| DDR1 | Epithelial discoidin domain-containing receptor 1 |
| LPL | Lipoprotein lipase |
| ABCC1 | Multidrug resistance-associated protein 1 |
| ASIC1 | Acid-sensing ion channel 1 |
| SLC2A1 | Solute carrier family 2, facilitated glucose transporter member 1 |
| PTPRS | Receptor-type tyrosine-protein phosphatase S |
| ABCG2 | Broad substrate specificity ATP-binding cassette transporter ABCG2 |
| SLC1A3 | Excitatory amino acid transporter 1 |
| SLC1A1 | Excitatory amino acid transporter 3 |
| TRPM2 | Transient receptor potential cation channel subfamily M member 2 |
| GRM1 | Metabotropic glutamate receptor 1 |
| MLKL | Mixed lineage kinase domain-like protein |
| RAB7A | Ras-related protein Rab-7a |
| AHCYL1 | S-adenosylhomocysteine hydrolase-like protein 1 |
| NAAA | N-acylethanolamine-hydrolyzing acid amidase |
| CAMK2B | Calcium/calmodulin-dependent protein kinase type II subunit beta |
| NT5E | 5′-nucleotidase |
| CTSL | Procathepsin L |
| HSPA1A | Heat shock 70 kDa protein 1A |
| FABP5 | Fatty acid-binding protein 5 |
| CTSC | Dipeptidyl peptidase 1 |
| ACVR2A | Activin receptor type-2A |
| MMP14 | Matrix metalloproteinase-14 |
| HSP90B1 | Endoplasmin |
| GLB1 | Beta-galactosidase |
| PTPRF | Receptor-type tyrosine-protein phosphatase F |
| ST14 | Suppressor of tumorigenicity 14 protein |
| PKN2 | Serine/threonine-protein kinase N2 |
| ARG1 | Arginase-1 |
| PGAM1 | Phosphoglycerate mutase 1 |
| PPP1CA | Serine/threonine-protein phosphatase PP1-alpha catalytic subunit |
| PTP4A2 | Protein tyrosine phosphatase type IVA 2 |
| MAST1 | Microtubule-associated serine/threonine-protein kinase 1 |
| PI4KB | Phosphatidylinositol 4-kinase beta |
| VRK2 | Serine/threonine-protein kinase VRK2 |
| SCD | Stearoyl-CoA desaturase |
| NUDT1 | 7,8-dihydro-8-oxoguanine triphosphatase |
| CSNK1G2 | Casein kinase I isoform gamma-2 |
| EBP | 3-beta-hydroxysteroid-Delta(8), Delta(7)-isomerase |
| EIF2AK3 | Eukaryotic translation initiation factor 2-alpha kinase 3 |
| ATP2A2 | Sarcoplasmic/endoplasmic reticulum calcium ATPase 2 |
| MAN1B1 | Endoplasmic reticulum mannosyl-oligosaccharide 1,2-alpha-mannosidase |
| CDKL5 | Cyclin-dependent kinase-like 5 |
| MAP4K4 | Mitogen-activated protein kinase kinase kinase kinase 4 |
| DPP8 | Dipeptidyl peptidase 8 |
| MKNK1 | MAP kinase-interacting serine/threonine-protein kinase 1 |
| PREP | Prolyl endopeptidase |
| FKBP5 | Peptidyl-prolyl cis-trans isomerase FKBP5 |
| SIRT1 | NAD-dependent protein deacetylase sirtuin-1 |
| STK3 | Serine/threonine-protein kinase 3 |
| LIG1 | DNA ligase 1 |
| DUSP3 | Dual specificity protein phosphatase 3 |
| CAMKK2 | Calcium/calmodulin-dependent protein kinase kinase 2 |
| PRKAB1 | 5′-AMP-activated protein kinase subunit beta-1 |
| MTR | Methionine synthase |
| UCHL3 | Ubiquitin carboxyl-terminal hydrolase isozyme L3 |
| ESRRB | Steroid hormone receptor ERR2 |
| RIOK1 | Serine/threonine-protein kinase RIO1 |
| TRAP1 | Heat shock protein 75 kDa, mitochondrial |
| PIN1 | Peptidyl-prolyl cis-trans isomerase NIMA-interacting 1 |
| ALDH1A2 | Retinal dehydrogenase 2 |
| PAK6 | Serine/threonine-protein kinase PAK 6 |
| AOX1 | Aldehyde oxidase |
| DPP9 | Dipeptidyl peptidase 9 |
| ACP1 | Low molecular weight phosphotyrosine protein phosphatase |
| PTPN7 | Tyrosine-protein phosphatase non-receptor type 7 |
| TLK2 | Serine/threonine-protein kinase tousled-like 2 |
| SMG1 | Serine/threonine-protein kinase SMG1 |
| YWHAZ | 14-3-3 protein zeta/delta |
| ALDH1A1 | Retinal dehydrogenase 1 |
| PHF8 | Histone lysine demethylase PHF8 |
| PRKAG1 | 5′-AMP-activated protein kinase subunit gamma-1 |
| PRMT3 | Protein arginine N-methyltransferase 3 |
| SF3B3 | Splicing factor 3B subunit 3 |
| AKT1S1 | Proline-rich AKT1 substrate 1 |
| CHUK | Inhibitor of nuclear factor kappa-B kinase subunit alpha |
| RPL36A | 60S ribosomal protein L36a |
| RPL31 | 60S ribosomal protein L31 |
| RPS16 | 40S ribosomal protein S16 |
| RPS19 | 40S ribosomal protein S19 |
| RPS12 | 40S ribosomal protein S12 |
| RPL22 | 60S ribosomal protein L22 |
| RPS25 | 40S ribosomal protein S25 |
| RPL21 | 60S ribosomal protein L21 |
| RPL23 | 60S ribosomal protein L23 |
| RPS4Y1 | 40S ribosomal protein S4, Y isoform 1 |
| RPL36 | 60S ribosomal protein L36 |
| RPL27 | 60S ribosomal protein L27 |
| RPS15A | 40S ribosomal protein S15a |
| RPS11 | 40S ribosomal protein S11 |
| RPL13A | 60S ribosomal protein L13a |
| RPL37 | 60S ribosomal protein L37 |
| RPL7 | 60S ribosomal protein L7 |
| RPL29 | 60S ribosomal protein L29 |
| RPL27A | 60S ribosomal protein L27a |
| RPL35A | 60S ribosomal protein L35a |
| RPS26 | 40S ribosomal protein S26 |
| RPL37A | 60S ribosomal protein L37a |
| RPS29 | 40S ribosomal protein S29 |
| RPS28 | 40S ribosomal protein S28 |
| RPL17 | 60S ribosomal protein L17 |
| FAU | 40S ribosomal protein S30 |
| SLC2A3 | Solute carrier family 2, facilitated glucose transporter member 3 |
| ASAH1 | Acid ceramidase |
| BTN3A1 | Butyrophilin subfamily 3 member A1 |
| NMT2 | Glycylpeptide N-tetradecanoyltransferase 2 |
| PI4KA | Phosphatidylinositol 4-kinase alpha |
| GNG2 | Guanine nucleotide-binding protein G(I)/G(S)/G(O) subunit gamma-2 |
| GNA11 | Guanine nucleotide-binding protein subunit alpha-11 |
| PRMT8 | Protein arginine N-methyltransferase 8 |
| LYPLA1 | Acyl-protein thioesterase 1 |
| LGALS3 | Galectin-3 |
| UBE2D3 | Ubiquitin-conjugating enzyme E2 D3 |
| S100A4 | Protein S100-A4 |
| LGALS9 | Galectin-9 |
| PIP4K2A | Phosphatidylinositol 5-phosphate 4-kinase type-2 alpha |
| LGMN | Legumain |
| GNB1 | Guanine nucleotide-binding protein G(I)/G(S)/G(T) subunit beta-1 |
| HINT1 | Histidine triad nucleotide-binding protein 1 |
| FTO | Alpha-ketoglutarate-dependent dioxygenase FTO |
| BRPF1 | Peregrin |
| ST6GAL1 | Beta-galactoside alpha-2,6-sialyltransferase 1 |
| INPPL1 | Phosphatidylinositol 3,4,5-trisphosphate 5-phosphatase 2 |
| PI4K2B | Phosphatidylinositol 4-kinase type 2-beta |
| PARP16 | Protein mono-ADP-ribosyltransferase PARP16 |
| HEXA | Beta-hexosaminidase subunit alpha |
| SGPL1 | Sphingosine-1-phosphate lyase 1 |
| MGAT2 | Alpha-1,6-mannosyl-glycoprotein 2-beta-N-acetylglucosaminyltransferase |
| LGALS8 | Galectin-8 |
| ASH2L | Set1/Ash2 histone methyltransferase complex subunit ASH2 |
| SARM1 | NAD(+) hydrolase SARM1 |
| LYPLA2 | Acyl-protein thioesterase 2 |
| ASNS | Asparagine synthetase [glutamine-hydrolyzing] |
| PABPC1 | Polyadenylate-binding protein 1 |
| PRMT5 | Protein arginine N-methyltransferase 5 |
| DNPH1 | 2′-deoxynucleoside 5′-phosphate N-hydrolase 1 |
| WDR48 | WD repeat-containing protein 48 |
| WDR77 | Methylosome protein 50 |
| ATAD2B | ATPase family AAA domain-containing protein 2B |
| COPS5 | COP9 signalosome complex subunit 5 |
| PFKFB2 | 6-phosphofructo-2-kinase/fructose-2,6-bisphosphatase 2 |
| KDM4B | Lysine-specific demethylase 4B |
| PRKAB2 | 5′-AMP-activated protein kinase subunit beta-2 |
| MEN1 | Menin |
| PMM2 | Phosphomannomutase 2 |
| RPTOR | Regulatory-associated protein of mTOR |
| MLST8 | Target of rapamycin complex subunit LST8 |
| DTYMK | Thymidylate kinase |
| PTPN9 | Tyrosine-protein phosphatase non-receptor type 9 |
| SHMT2 | Serine hydroxymethyltransferase, mitochondrial |
| PARG | Poly(ADP-ribose) glycohydrolase |
| GLS | Glutaminase kidney isoform, mitochondrial |
| APAF1 | Apoptotic protease-activating factor 1 |
| KSR2 | Kinase suppressor of Ras 2 |
| SORD | Sorbitol dehydrogenase |
| SMS | Spermine synthase |
| GALE | UDP-glucose 4-epimerase |
| PGD | 6-phosphogluconate dehydrogenase, decarboxylating |
| HNMT | Histamine N-methyltransferase |
| QDPR | Dihydropteridine reductase |
| GSTK1 | Glutathione S-transferase kappa 1 |
| SRM | Spermidine synthase |
| FARS2 | Phenylalanine--tRNA ligase, mitochondrial |
| GNAI3 | Guanine nucleotide-binding protein G(i) subunit alpha-3 |
| CLTC | Clathrin heavy chain 1 |
| RHOC | Rho-related GTP-binding protein RhoC |
| DNM1 | Dynamin-1 |
| GALNT10 | Polypeptide N-acetylgalactosaminyltransferase 10 |
| MTHFD1 | C-1-tetrahydrofolate synthase, cytoplasmic |
| RAB2A | Ras-related protein Rab-2A |
| USP5 | Ubiquitin carboxyl-terminal hydrolase 5 |
| GDA | Guanine deaminase |
| UCK2 | Uridine-cytidine kinase 2 |
| HIF1AN | Hypoxia-inducible factor 1-alpha inhibitor |
| MDH2 | Malate dehydrogenase, mitochondrial |
| CYP2E1 | Cytochrome P450 2E1 |
| YWHAG | 14-3-3 protein gamma |
| GFPT1 | Glutamine--fructose-6-phosphate aminotransferase [isomerizing] 1 |
| FN1 | Fibronectin |
| PLAUR | Urokinase plasminogen activator surface receptor |
| NCS1 | Neuronal calcium sensor 1 |
| GNAI1 | Guanine nucleotide-binding protein G(i) subunit alpha-1 |
| HCN2 | Potassium/sodium hyperpolarization-activated cyclic nucleotide-gated |
| | channel 2 |
| QSOX1 | Sulfhydryl oxidase 1 |
| TLR1 | Toll-like receptor 1 |
| USP14 | Ubiquitin carboxyl-terminal hydrolase 14 |
| KSR1 | Kinase suppressor of Ras 1 |
| RAB9A | Ras-related protein Rab-9A |
| CCR7 | C-C chemokine receptor type 7 |
| PLCG2 | 1-phosphatidylinositol 4,5-bisphosphate phosphodiesterase gamma-2 |
| GFER | FAD-linked sulfhydryl oxidase ALR |
| PTPRG | Receptor-type tyrosine-protein phosphatase gamma |
| FUT8 | Alpha-(1,6)-fucosyltransferase |
| POR | NADPH-cytochrome P450 reductase |
| RECQL | ATP-dependent DNA helicase Q1 |
| UNG | Uracil-DNA glycosylase |
| CAD | CAD protein [Includes: Glutamine-dependent carbamoyl-phosphate |
| | synthase |
| NCOA3 | Nuclear receptor coactivator 3 |
| CTDSP1 | Carboxy-terminal domain RNA polymerase II polypeptide A small |
| | phosphatase 1 |
| KDM1B | Lysine-specific histone demethylase 1B |
| TEAD1 | Transcriptional enhancer factor TEF-1 |
| DDO | D-aspartate oxidase |
| CD4 | T-cell surface glycoprotein CD4 |
| SLC1A5 | Neutral amino acid transporter B(0) |
| CD81 | CD81 antigen |
| GGT1 | Glutathione hydrolase 1 proenzyme |
| ADIPOR1 | Adiponectin receptor protein 1 |
| RAP1A | Ras-related protein Rap-1A |
| RAB29 | Ras-related protein Rab-7L1 |
| GOPC | Golgi-associated PDZ and coiled-coil motif-containing protein |
| UTRN | Utrophin |
| GNAO1 | Guanine nucleotide-binding protein G(o) subunit alpha |
| DDOST | Dolichyl-diphosphooligosaccharide--protein glycosyltransferase 48 kDa |
| | subunit |
| PYGB | Glycogen phosphorylase, brain form |
| RABGGTB | Geranylgeranyl transferase type-2 subunit beta |
| VAV1 | Proto-oncogene vav |
| SDHA | Succinate dehydrogenase [ubiquinone] flavoprotein subunit, mitochondrial |
| QTRT1 | Queuine tRNA-ribosyltransferase catalytic subunit 1 |
| CISD2 | CDGSH iron-sulfur domain-containing protein 2 |
| DHPS | Deoxyhypusine synthase |
| PPP2R5A | Serine/threonine-protein phosphatase 2A 56 kDa regulatory subunit alpha |
| | isoform |
| PANK2 | Pantothenate kinase 2, mitochondrial |
| NEDD8 | NEDD8 |
| DOHH | Deoxyhypusine hydroxylase |
| UBE2M | NEDD8-conjugating enzyme Ubc12 |
| RNASEL | 2-5A-dependent ribonuclease |
| SYNJ1 | Synaptojanin-1 |
| DUS2 | tRNA-dihydrouridine(20) synthase [NAD(P)+]-like |
| CYCS | Cytochrome c |
| PPP1CC | Serine/threonine-protein phosphatase PP1-gamma catalytic subunit |
| HMBS | Porphobilinogen deaminase |
| PNPO | Pyridoxine-5′-phosphate oxidase |
| PPIF | Peptidyl-prolyl cis-trans isomerase F, mitochondrial |
| DPP7 | Dipeptidyl peptidase 2 |
| MARK1 | Serine/threonine-protein kinase MARK1 |
| PRCP | Lysosomal Pro-X carboxypeptidase |
| PIP4K2C | Phosphatidylinositol 5-phosphate 4-kinase type-2 gamma |
| AKR1A1 | Aldo-keto reductase family 1 member A1 |
| ME1 | NADP-dependent malic enzyme |
| ACACA | Acetyl-CoA carboxylase 1 |
| CD74 | HLA class II histocompatibility antigen gamma chain |
| NFATC1 | Nuclear factor of activated T-cells, cytoplasmic 1 |
| RUNX1 | Runt-related transcription factor 1 |
| PTPRC | Receptor-type tyrosine-protein phosphatase C |
| CAPN2 | Calpain-2 catalytic subunit |
| RB1 | Retinoblastoma-associated protein |
| ITGA5 | Integrin alpha-5 |
| ITGA2 | Integrin alpha-2 |
| ADAM10 | Disintegrin and metalloproteinase domain-containing protein 10 |
| DAGLB | Diacylglycerol lipase-beta |
| SLC16A1 | Monocarboxylate transporter 1 |
| ABCC2 | ATP-binding cassette sub-family C member 2 |
| SCARB1 | Scavenger receptor class B member 1 |
| DAGLA | Diacylglycerol lipase-alpha |
| SLC47A1 | Multidrug and toxin extrusion protein 1 |
| PIK3C2B | Phosphatidylinositol 4-phosphate 3-kinase C2 domain-containing subunit |
| | beta |
| GBA2 | Non-lysosomal glucosylceramidase |
| NCEH1 | Neutral cholesterol ester hydrolase 1 |
| WNK2 | Serine/threonine-protein kinase WNK2 |
| LIPE | Hormone-sensitive lipase |
| SLC27A1 | Long-chain fatty acid transport protein 1 |
| PTPRA | Receptor-type tyrosine-protein phosphatase alpha |
| PTPN2 | Tyrosine-protein phosphatase non-receptor type 2 |
| ITGB5 | Integrin beta-5 |
| CTSH | Pro-cathepsin H [Cleaved into: Cathepsin H mini chain; Cathepsin H |
| MAN2B1 | Lysosomal alpha-mannosidase |
| ITPR2 | Inositol 1,4,5-trisphosphate receptor type 2 |
| ITPR3 | Inositol 1,4,5-trisphosphate receptor type 3 |
| ABHD12 | Lysophosphatidylserine lipase ABHD12 |
| CTSZ | Cathepsin Z |
| SLC6A12 | Sodium- and chloride-dependent betaine transporter |
| SLC6A11 | Sodium- and chloride-dependent GABA transporter 3 |
| KCNN3 | Small conductance calcium-activated potassium channel protein 3 |
| SLC27A4 | Long-chain fatty acid transport protein 4 |
| PTPN6 | Tyrosine-protein phosphatase non-receptor type 6 |
| AMPD3 | AMP deaminase 3 |
| PLEC | Plectin |
| PLD1 | Phospholipase D1 |
| PAM | Peptidyl-glycine alpha-amidating monooxygenase |
| PRKD2 | Serine/threonine-protein kinase D2 |
| TYRO3 | Tyrosine-protein kinase receptor TYRO3 |
| RYR2 | Ryanodine receptor 2 |
| PIP5K1C | Phosphatidylinositol 4-phosphate 5-kinase type-1 gamma |
| LIPA | Lysosomal acid lipase/cholesteryl ester hydrolase |
| PPIB | Peptidyl-prolyl cis-trans isomerase B |
| ATP2A3 | Sarcoplasmic/endoplasmic reticulum calcium ATPase 3 |
| CPT1A | Carnitine O-palmitoyltransferase 1, liver isoform |
| ICMT | Protein-S-isoprenylcysteine O-methyltransferase |
| HSD17B7 | 3-keto-steroid reductase/17-beta-hydroxysteroid dehydrogenase 7 |
| ELOVL6 | Elongation of very long chain fatty acids protein 6 |
| DHCR7 | 7-dehydrocholesterol reductase |
| ATP2A1 | Sarcoplasmic/endoplasmic reticulum calcium ATPase 1 |
| RIOK3 | Serine/threonine-protein kinase RIO3 |
| HSPB1 | Heat shock protein beta-1 |
| PTPN12 | Tyrosine-protein phosphatase non-receptor type 12 |
| PLAA | Phospholipase A-2-activating protein |
| MPI | Mannose-6-phosphate isomerase |
| MATK | Megakaryocyte-associated tyrosine-protein kinase |
| UBA6 | Ubiquitin-like modifier-activating enzyme 6 |
| MAP2K3 | Dual specificity mitogen-activated protein kinase kinase 3 |
| RPS6KA2 | Ribosomal protein S6 kinase alpha-2 |
| MAP3K4 | Mitogen-activated protein kinase kinase kinase 4 |
| PARP4 | Protein mono-ADP-ribosyltransferase PARP4 |
| EIF4H | Eukaryotic translation initiation factor 4H |
| TESK1 | Dual specificity testis-specific protein kinase 1 |
| MLX | Max-like protein X |
| NEK6 | Serine/threonine-protein kinase Nek6 |
| ALDH1B1 | Aldehyde dehydrogenase X, mitochondrial |
| ATM | Serine-protein kinase ATM |
| CDYL | Chromodomain Y-like protein |
| TAOK1 | Serine/threonine-protein kinase TAO1 |
| STAT6 | Signal transducer and activator of transcription 6 |
| MGMT | Methylated-DNA -- protein-cysteine methyltransferase |
| PPID | Peptidyl-prolyl cis-trans isomerase D |
| STAT5B | Signal transducer and activator of transcription 5B |
| TXNRD2 | Thioredoxin reductase 2, mitochondrial |
| TXNRD3 | Thioredoxin reductase 3 |
| STK39 | STE20/SPS1-related proline-alanine-rich protein kinase |
| STK38L | Serine/threonine-protein kinase 38-like |
| CDC42BPA | Serine/threonine-protein kinase MRCK alpha |
| ATG4B | Cysteine protease ATG4B |
| DCLK2 | Serine/threonine-protein kinase DCLK2 |
| TUBAL3 | Tubulin alpha chain-like 3 |
| MAP3K3 | Mitogen-activated protein kinase kinase kinase 3 |
| PPME1 | Protein phosphatase methylesterase 1 |
| RPLP1 | 60S acidic ribosomal protein P1 |
| GSDMD | Gasdermin-D |
| C9 | Complement component C9 [Cleaved into: Complement component C9a; |
| | Complement component C9b] |
| HMGB1 | High mobility group protein B1 |
| DAB2IP | Disabled homolog 2-interacting protein |
| PHLPP1 | PH domain leucine-rich repeat-containing protein phosphatase 1 |
| PHKA2 | Phosphorylase b kinase regulatory subunit alpha, liver isoform |
| PHKA1 | Phosphorylase b kinase regulatory subunit alpha, skeletal muscle isoform |
| PHKB | Phosphorylase b kinase regulatory subunit beta |
| EWSR1 | RNA-binding protein EWS |
| SLC16A3 | Monocarboxylate transporter 4 |
| PTPRE | Receptor-type tyrosine-protein phosphatase epsilon |
| NEU1 | Sialidase-1 |
| PCSK6 | Proprotein convertase subtilisin/kexin type 6 |
| ANTXR2 | Anthrax toxin receptor 2 |
| STIM1 | Stromal interaction molecule 1 |
| DNM2 | Dynamin-2 |
| SDHB | Succinate dehydrogenase [ubiquinone] iron-sulfur subunit, mitochondrial |
| MAPKAP1 | Target of rapamycin complex 2 subunit MAPKAP1 |
| PTGES2 | Prostaglandin E synthase 2 |
| TMEM97 | Sigma intracellular receptor 2 |
| SPPL2A | Signal peptide peptidase-like 2A |
| SLC7A11 | Cystine/glutamate transporter |
| ABHD6 | Monoacylglycerol lipase ABHD6 |
| DGKA | Diacylglycerol kinase alpha |
| NAPRT | Nicotinate phosphoribosyltransferase |
| BAX | Apoptosis regulator BAX |
| SPTLC1 | Serine palmitoyltransferase 1 |
| SPTLC2 | Serine palmitoyltransferase 2 |
| MGAT1 | Alpha-1,3-mannosyl-glycoprotein 2-beta-N-acetylglucosaminyltransferase |
| EPHX1 | Epoxide hydrolase 1 |
| FADS1 | Acyl-CoA |
| ABHD5 | 1-acylglycerol-3-phosphate O-acyltransferase ABHD5 |
| CBFB | Core-binding factor subunit beta |
| POLR1A | DNA-directed RNA polymerase I subunit RPA1 |
| MPG | DNA-3-methyladenine glycosylase |
| GYS1 | Glycogen [starch] synthase, muscle |
| PRKAG2 | 5′-AMP-activated protein kinase subunit gamma-2 |
| ASF1A | Histone chaperone ASF1A |
| MED23 | Mediator of RNA polymerase II transcription subunit 23 |
| PFKFB4 | 6-phosphofructo-2-kinase/fructose-2,6-bisphosphatase 4 |
| KDM3A | Lysine-specific demethylase 3A |
| USP9X | Probable ubiquitin carboxyl-terminal hydrolase FAF-X |
| MAX | Protein max |
| MBD2 | Methyl-CpG-binding domain protein 2 |
| CBX8 | Chromobox protein homolog 8 |
| FLI1 | Friend leukemia integration 1 transcription factor |
| ATRIP | ATR-interacting protein |
| RICTOR | Rapamycin-insensitive companion of mTOR |
| UBLCP1 | Ubiquitin-like domain-containing CTD phosphatase 1 |
| DCTPP1 | dCTP pyrophosphatase 1 |
| TCF4 | Transcription factor 4 |
| CNOT7 | CCR4-NOT transcription complex subunit 7 |
| OGFRL1 | Opioid growth factor receptor-like protein 1 |
| MVD | Diphosphomevalonate decarboxylase |
| IL6ST | Interleukin-6 receptor subunit beta |
| DOCK2 | Dedicator of cytokinesis protein 2 |
| C3 | Complement C3 |
| PPM1A | Protein phosphatase 1A |
| SHC1 | SHC-transforming protein 1 |
| DVL2 | Segment polarity protein dishevelled homolog DVL-2 |
| NPR3 | Atrial natriuretic peptide receptor 3 |
| GSTM1 | Glutathione S-transferase Mu 1 |
| PPP3CA | Serine/threonine-protein phosphatase 2B catalytic subunit alpha isoform |
| SLC12A2 | Solute carrier family 12 member 2 |
| ABCC3 | ATP-binding cassette sub-family C member 3 |
| ABCC4 | ATP-binding cassette sub-family C member 4 |
| GGH | Gamma-glutamyl hydrolase |
| HCN3 | Potassium/sodium hyperpolarization-activated cyclic nucleotide-gated |
| | channel 3 |
| ADCY1 | Adenylate cyclase type 1 |
| ADCY5 | Adenylate cyclase type 5 |
| CYP4F2 | Cytochrome P450 4F2 |
| HKDC1 | Hexokinase HKDC1 |
| PPT1 | Palmitoyl-protein thioesterase 1 |
| PRNP | Major prion protein |
| CXCR5 | C-X-C chemokine receptor type 5 |
| SMPD2 | Sphingomyelin phosphodiesterase 2 |
| SLC6A15 | Sodium-dependent neutral amino acid transporter B(0)AT2 |
| MANBA | Beta-mannosidase |
| AGPAT2 | 1-acyl-sn-glycerol-3-phosphate acyltransferase beta |
| ABHD16A | Phosphatidylserine lipase ABHD16A |
| GCLC | Glutamate-cysteine ligase catalytic subunit |
| HAGH | Hydroxyacylglutathione hydrolase, mitochondrial |
| SENP1 | Sentrin-specific protease 1 |
| DNAJA1 | DnaJ homolog subfamily A member 1 |
| SCP2 | Sterol carrier protein 2 |
| AMPD2 | AMP deaminase 2 |
| ERCC5 | DNA repair protein complementing XP-G cells |
| SENP7 | Sentrin-specific protease 7 |
| RBPJ | Recombining binding protein suppressor of hairless |
| USP47 | Ubiquitin carboxyl-terminal hydrolase 47 |
| NADK | NAD kinase |
| RBBP9 | Serine hydrolase RBBP9 |
| CD2 | T-cell surface antigen CD2 |
| CD44 | CD44 antigen |
| SLC27A2 | Very long-chain acyl-CoA synthetase |
| PLEKHA1 | Pleckstrin homology domain-containing family A member 1 |
| ITGA3 | Integrin alpha-3 |
| CD47 | Leukocyte surface antigen CD47 |
| SPPL2B | Signal peptide peptidase-like 2B |
| PLTP | Phospholipid transfer protein |
| CD63 | CD63 antigen |
| SLC20A2 | Sodium-dependent phosphate transporter 2 |
| ADCY6 | Adenylate cyclase type 6 |
| IGF2R | Cation-independent mannose-6-phosphate receptor |
| EZR | Ezrin |
| GNAQ | Guanine nucleotide-binding protein G(q) subunit alpha |
| INPP5A | Inositol polyphosphate-5-phosphatase A |
| CRACR2A | EF-hand calcium-binding domain-containing protein 4B |
| LANCL2 | LanC-like protein 2 |
| SMPD3 | Sphingomyelin phosphodiesterase 3 |
| EHD1 | EH domain-containing protein 1 |
| PDIA6 | Protein disulfide-isomerase A6 |
| CHN2 | Beta-chimaerin |
| CDH2 | Cadherin-2 |
| TFPI | Tissue factor pathway inhibitor |
| CD58 | Lymphocyte function-associated antigen 3 |
| SLC2A8 | Solute carrier family 2, facilitated glucose transporter member 8 |
| PTPRM | Receptor-type tyrosine-protein phosphatase mu |
| STIM2 | Stromal interaction molecule 2 |
| ADCY7 | Adenylate cyclase type 7 |
| SLC20A1 | Sodium-dependent phosphate transporter 1 |
| ITGA1 | Integrin alpha-1 |
| RABGGTA | Geranylgeranyl transferase type-2 subunit alpha |
| MAP1B | Microtubule-associated protein 1B |
| PAFAH1B2 | Platelet-activating factor acetylhydrolase IB subunit alpha2 |
| RIN1 | Ras and Rab interactor 1 |
| PLCD1 | 1-phosphatidylinositol 4,5-bisphosphate phosphodiesterase delta-1 |
| RGS19 | Regulator of G-protein signaling 19 |
| PNPLA2 | Patatin-like phospholipase domain-containing protein 2 |
| ST3GAL3 | CMP-N-acetylneuraminate-beta-1,4-galactoside alpha-2,3-sialyltransferase |
| GALNT1 | Polypeptide N-acetylgalactosaminyltransferase 1 |
| ATP6V1B2 | V-type proton ATPase subunit B, brain isoform |
| ATP6AP1 | V-type proton ATPase subunit S1 |
| PHLPP2 | PH domain leucine-rich repeat-containing protein phosphatase 2 |
| GNPAT | Dihydroxyacetone phosphate acyltransferase |
| PPP3CB | Serine/threonine-protein phosphatase 2B catalytic subunit beta isoform |
| SLC25A20 | Mitochondrial carnitine/acylcarnitine carrier protein |
| ELOVL1 | Elongation of very long chain fatty acids protein 1 |
| MAN2A1 | Alpha-mannosidase 2 |
| SLC33A1 | Acetyl-coenzyme A transporter 1 |
| GAB1 | GRB2-associated-binding protein 1 |
| ARHGEF12 | Rho guanine nucleotide exchange factor 12 |
| MAP2 | Microtubule-associated protein 2 |
| MBTD1 | MBT domain-containing protein 1 |
| PNKP | Bifunctional polynucleotide phosphatase/kinase |
| NLRP1 | NACHT, LRR and PYD domains-containing protein 1 |
| TXN2 | Thioredoxin, mitochondrial |
| EEF1A2 | Elongation factor 1-alpha 2 |
| GOT1 | Aspartate aminotransferase, cytoplasmic |
| PRMT7 | Protein arginine N-methyltransferase 7 |
| MYH10 | Myosin-10 |
| TPP2 | Tripeptidyl-peptidase 2 |
| EYA3 | Eyes absent homolog 3 |
| SSU72 | RNA polymerase II subunit A C-terminal domain phosphatase SSU72 |
| AMDHD2 | N-acetylglucosamine-6-phosphate deacetylase |
| CLPX | ATP-dependent Clp protease ATP-binding subunit clpX-like, mitochondrial |
| MECP2 | Methyl-CpG-binding protein 2 |
| KPNA2 | Importin subunit alpha-1 |
| PSMG1 | Proteasome assembly chaperone 1 |
| CBX6 | Chromobox protein homolog 6 |
| PPP2R2A | Serine/threonine-protein phosphatase 2A 55 kDa regulatory subunit B alpha |
| | isoform |
| UBE2V1 | Ubiquitin-conjugating enzyme E2 variant 1 |
| PSMG2 | Proteasome assembly chaperone 2 |
| PPIC | Peptidyl-prolyl cis-trans isomerase C |
| PGK2 | Phosphoglycerate kinase 2 |

In some embodiments, the PBM is a small molecule ligand that targets specific target proteins including but not limited to EGFR, CDK4/6, ALK, RET, FAK, BCR-ABL, BTK, AR, ER or BET.

In some embodiments, the PBM comprises the structures of the following formula:

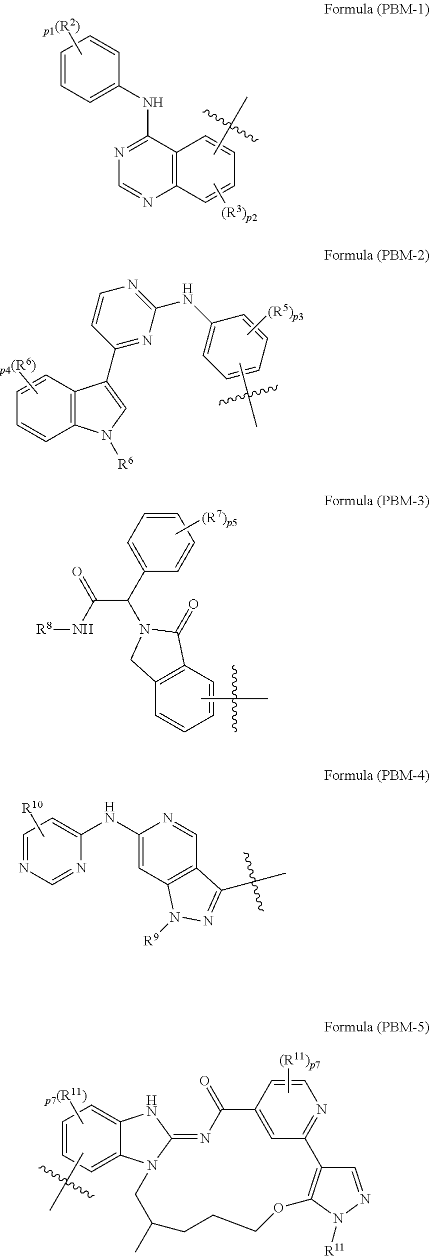

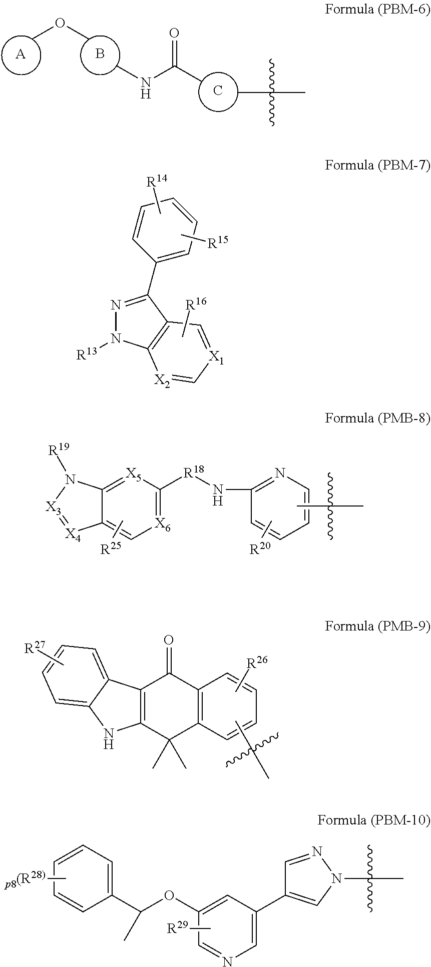

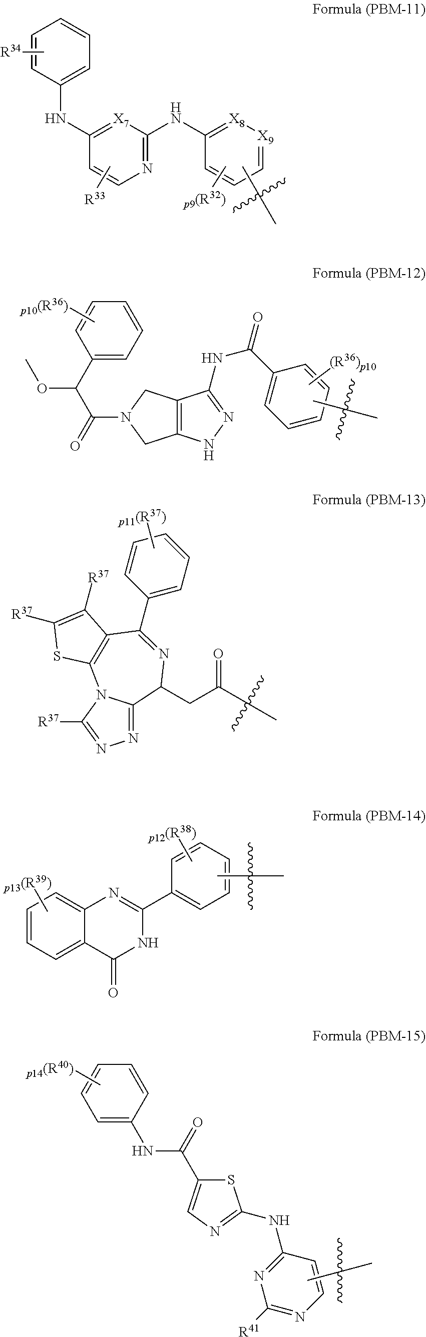

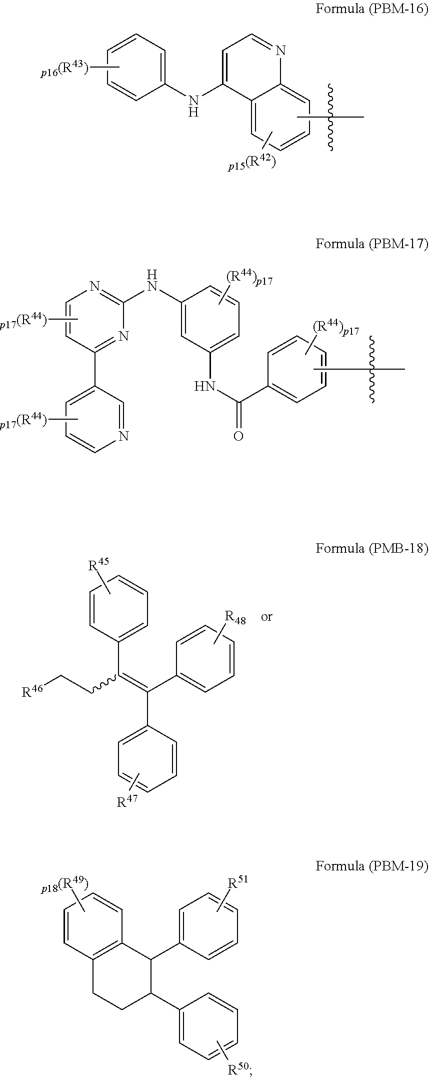

wherein
(R²)ₚ₁ indicates that the benzene ring in Formula (PBM-1) is substituted with p1 R², and each R² are the same or different and each independently represent halogen or halogenated C₁₋₆ alkyl;
(R³)ₚ₂ indicates that the quinazoline ring in Formula (PBM-1) is substituted with p2 R³, and each R³ are the same or different and each independently represent C₁₋₁₀ alkyl, deuterated C₁₋₁₀ alkyl, C₁₋₁₀ alkoxy, heterocyclyloxy, or —NHC(O)R⁴, wherein R⁴ is C₁₋₁₀ alkyl or C₂₋₁₀ alkenyl, e.g., R³ represents methyl, methoxy,

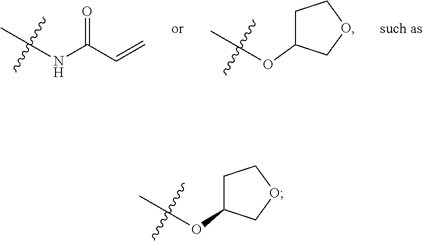

p1 represents an integer of 1, 2, 3, 4 or 5;
p2 represents an integer of 1, 2, or 3;
(R⁵)ₚ₃ indicates that the benzene ring in Formula (PBM-2) is substituted with p3 R⁵, and each R⁵ are the same or different and each independently represent C₁₋₁₀ alkyl, deuterated C₁₋₁₀ alkyl, C₁₋₁₀ alkoxy, or —NHC(O)R⁴, wherein R⁴ is C₁₋₁₀ alkyl, deuterated C₁₋₁₀ alkyl or C₂₋₁₀ alkenyl, e.g., represents methyl, methoxy or

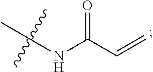 [SMILES: *NC(=O)C=C]

p3 represents an integer of 1, 2, 3 or 4;
(R⁶)P₄ indicates that the 1H-indole ring in Formula (PBM-2) is substituted with p4 R⁶, and each R⁶ are the same or different and each independently represent C₁₋₁₀ alkyl or deuterated C₁₋₁₀ alkyl;
p4 represents an integer of 0, 1, 2 or 3;
(R⁷)ₚ₅ indicates that the benzene ring in Formula (PBM-3) is substituted with p5 R⁷, and each R⁷ are the same or different and each independently represent halogen, hydroxy, C₁₋₆ alkyl, halogenated C₁₋₆ alkyl, deuterated C₁₋₆ alkyl, NO₂, NH₂, or cyano;
p5 represents an integer of 2, 3, 4 or 5;
R⁸ represents C₆₋₁₀ aryl or heteroaryl containing one or two 5-to 7-membered rings and 1 to 4 heteroatoms selected from nitrogen, oxygen, and sulfur, wherein the C₆₋₁₀ aryl and heteroaryl are each optionally substituted with one or more R^{4a}, and each R^{4a} are the same or different and are each independently halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy;
R⁹ represents C₁₋₁₀ alkyl (e.g.

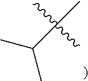 [SMILES: *C(C)C]

or deuterated C₁₋₁₀ alkyl;
R¹⁰ represents

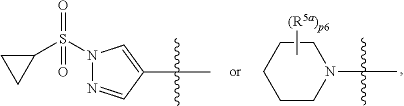

wherein (R^{5a})ₚ₆ indicates that the piperidine ring is substituted with p6 R^{5a}, and each R^{5a} are the same or different and each independently represent deuterium, halogen, hydroxy, C₁₋₆ alkyl, halogenated C₁₋₆ alkyl, deuterated C₁₋₆ alkyl, C₁₋₆ alkoxy, deuterated C₁₋₆ alkoxy, NO₂, NH₂, or cyano, p6 represents an integer of 1, 2, 3, 4 or 5;
(R¹¹)ₚ₇ indicates that the benzene ring and the pyridine ring in Formula (PBM-5) are each optionally substituted with p7 R¹¹, and each R¹¹ are the same or different and each independently represent halogen, C₁₋₆ alkyl, halogenated C₁₋₆ alkyl, or deuterated C₁₋₆ alkyl, p7 represents an integer of 0, 1, 2 or 3;
ring A in Formula (PBM-6) represents C₆₋₁₀ aryl or heteroaryl containing one or two 5- to 7-membered rings and from 1 to 4 heteroatoms selected from the group consisting of oxygen, nitrogen, and sulfur, and the ring A is optionally substituted with one or more R^{6a}, and each R^{6a} are the same or different and each independently represent deuterium, halogen, hydroxy, cyano, amino, nitro, C₁₋₆ alkyl, halogenated C₁₋₆ alkyl, deuterated C₁₋₆ alkyl, C₁₋₆ alkoxy, halogenated C₁₋₆ alkoxy, C₂₋₆ alkenyl, or C₂₋₆ alkynyl;
ring B in Formula (PBM-6) represents C₃₋₁₅ cycloalkyl or heterocyclyl containing one or two 3 to 7-membered rings and from 1 to 4 heteroatoms selected from the group consisting of oxygen, nitrogen, and sulfur, and the ring B is optionally substituted with one or more R^{7a}, and each R^{7a} are the same or different and each independently represent deuterium, halogen, C₁₋₆ alkyl, halogenated C₁₋₆ alkyl, deuterated C₁₋₆ alkyl, C₁₋₆ alkoxy, or halogenated C₁₋₆ alkoxy;
ring C in Formula (PBM-6) represents heteroaryl containing one or two 5- to 7-membered rings and from 1 to 4 heteroatoms selected from the group consisting of oxygen, nitrogen, and sulfur, and the ring C is optionally substituted with one or more R^{8a}, and each R^{8a} are the same or different and each independently represent deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
X₁ in Formula (PBM-7) represents N or CR¹², wherein R¹² is hydrogen or represents a bond, and X₂ represents CH or N;
R¹³ is hydrogen or represents a bond, wherein when R¹³ represents a bond, X₁ represents N or CH, and the nitrogen atom in the ring connected to R¹³ is directly connected to R¹; or when R¹³ is hydrogen, X₁ represents CR¹², wherein R¹² represents a bond, and the carbon atom in the ring connected to R¹² is directly connected to R¹;
R¹⁴ represents —O-aryl, —O-heteroaryl, —C₁₋₃-alkylene-NHC(O)-heteroaryl, or —C₁₋₃-alkylene-C(O)NH-heteroaryl, wherein the aryl and heteroaryl are each independently optionally substituted with one or more R^{9a}, and each R^{9a} are the same or different and each independently represent deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
R¹⁵ represents deuterium, hydrogen, halogen, C₁₋₆ alkyl, halogenated C₁₋₆ alkyl, deuterated C₁₋₆ alkyl or —NHC(O)R¹⁷, wherein R¹⁷ is C₁₋₁₀ alkyl or C₂₋₁₀ alkenyl, e.g., R¹⁵ represents hydrogen, methyl, or

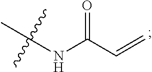 [SMILES: *NC(=O)C=C]

R¹⁶ represents hydrogen, cyano, or amino;
X₃ in Formula (PBM-8) represents CR²¹ or a fragment

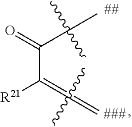

wherein symbol ## indicates the point of attachment to N atom adjacent to X₃, symbol ### indicates the point of attachment to X₄, and R²¹ represents deuterium, hydrogen, halogen, C₁₋₆ alkyl, halogenated C₁₋₆ alkyl, deuterated C₁₋₆ alkyl, —C(O)—C₁₋₆ alkyl, —C(O)-deuterated C₁₋₆ alkyl, or —C(O)NR²²R²³, wherein R²² and R²³ are the same or different and each independently represent hydrogen, C₁₋₆ alkyl or deuterated C₁₋₆ alkyl, and R²² and R²³ are not simultaneously hydrogen;
X₄ in Formula (PBM-8) represents N or CR²⁴, wherein R²⁴ represents hydrogen, deuterium, halogen, C₁₋₆ alkyl, deuterated C₁₋₆ alkyl or halogenated C₁₋₆ alkyl;
X₅ and X₆ each independently represents CH or N;
R¹⁸ represents a bond or optionally substituted heteroarylene (e.g., halogenated heteroarylene);
R¹⁹ represents hydrogen, C₁₋₆ alkyl, deuterated C₁₋₆ alkyl, halogenated C₁₋₆ alkyl, or optionally substituted C₃₋₇ cycloalkyl;
R²⁰ and R²⁵ are the same or different and each independently represent hydrogen, halogen, C₁₋₆ alkyl, deuterated C₁₋₆ alkyl or halogenated C₁₋₆ alkyl;
R²⁶ represents hydrogen, halogen, C₁₋₆ alkyl, deuterated C₁₋₆ alkyl or halogenated C₁₋₆ alkyl;
R²⁷ represents hydrogen, cyano, halogen, C₁₋₆ alkyl, deuterated C₁₋₆ alkyl or halogenated C₁₋₆ alkyl;
(R²)ₚ₈ indicates that the benzene ring in Formula (PBM-10) is substituted with p8 R²⁸ substituents, and each R²⁸ are the same or different and each independently represent halogen, C₁₋₆ alkyl, deuterated C₁₋₆ alkyl or halogenated C₁₋₆ alkyl;
p8 represents an integer of 1, 2, 3 or 4;
R²⁹ represents NR³⁰R³¹, wherein R³⁰ and R³¹ are the same or different and each independently represent hydrogen, C₁₋₆ alkyl, or deuterated C₁₋₆ alkyl;
X₇, X₈ and X₉ in Formula (PBM-11) are the same or different and each independently represent N or CH;
(R³²)ₚ₉ indicates that the 6-membered ring containing X₈ and X₉ in Formula (PBM-11) is optionally substituted with p9 R³² groups, and each R³² are the same or different and each independently represent halogen, C₁₋₆ alkoxy, C₁₋₆ alkyl, deuterated C₁₋₆ alkyl or halogenated C₁₋₆ alkyl;
p9 represents an integer of 0, 1, 2, 3 or 4;
R³³ represents halogen, C₁₋₆ alkoxy, C₁₋₆ alkyl, deuterated C₁₋₆ alkyl or halogenated C₁₋₆ alkyl;
R³⁴ represents —C(O)NHR³⁵, —NHC(O)—R³⁵, —S(O)₂NHR³⁵, —S(O)₂R³¹ or —P(O)(R³⁵)₂, wherein each R³⁵ are the same or different and each independently represent C₁₋₆ alkyl or deuterated C₁₋₆ alkyl;
(R³⁶)ₚ₁₀ indicates that the benzene ring in Formula (PBM-12) is optionally substituted with p10 R³⁶ groups, and each R³⁶ are the same or different and each independently represent halogen, C₁₋₆ alkoxy, C₁₋₆ alkyl, deuterated C₁₋₆ alkyl or halogenated C₁₋₆ alkyl;
p10 represents an integer of 0, 1, 2, 3 or 4;
(R³⁷)ₚ₁₁ indicates that the benzene ring in Formula (PBM-13) is substituted with p11 R³⁷ groups, and each R³⁷ are the same or different and each independently represent halogen, C₁₋₆ alkoxy, C₁₋₆ alkyl, deuterated C₁₋₆ alkyl or halogenated C₁₋₆ alkyl;
p11 represents an integer of 0, 1, 2, 3 or 4;
(R³⁸)ₚ₁₂ indicates that the benzene ring in Formula (PBM-14) is substituted with p12 R³⁸ groups, and each R³⁸ are the same or different and each independently represent halogen, C₁₋₆ alkoxy, C₁₋₆ alkyl, deuterated C₁₋₆ alkyl or halogenated C₁₋₆ alkyl;
p12 represents an integer of 0, 1, 2, 3 or 4;
(R³⁹)ₚ₁₃ indicates that the 4-oxo-3,4-dihydroquinazoline ring in Formula (PBM-14) is substituted with p13 R³⁹ groups, and each R³⁹ are the same or different and each independently represent halogen, C₁₋₆ alkoxy, C₁₋₆ alkyl, deuterated C₁₋₆ alkyl or halogenated C₁₋₆ alkyl;
p13 represents an integer of 1, 2, 3 or 4;
(R⁴⁰)ₚ₁₄ indicates that the benzene ring in Formula (PBM-15) is substituted with p14 R⁴⁰ groups, and each R⁴⁰ are the same or different and each independently represent halogen, C₁₋₆ alkoxy, C₁₋₆ alkyl, deuterated C₁₋₆ alkyl or halogenated C₁₋₆ alkyl;
p14 represents an integer of 1, 2, 3 or 4;
R⁴¹ represents halogen, C₁₋₆ alkyl, deuterated C₁₋₆ alkyl or halogenated C₁₋₆ alkyl;
(R⁴²)ₚ₁₅ indicates that the quinoline ring in Formula (PBM-16) is substituted with p15 R⁴² groups, and each R⁴² are the same or different and each independently represent halogen, cyano, C₁₋₆ alkoxy, C₁₋₆ alkyl, deuterated C₁₋₆ alkyl or halogenated C₁₋₆ alkyl;
p15 represents an integer of 2, 3 or 4;
(R⁴³)ₚ₁₆ indicates that the benzene ring in Formula (PBM-16) is substituted with p16 R⁴³ groups, and each R⁴³ are the same or different and each independently represent halogen, C₁₋₆ alkoxy, C₁₋₆ alkyl, deuterated C₁₋₆ alkyl or halogenated C₁₋₆ alkyl;
p16 represents an integer of 3 or 4;
(R⁴)ₚ₁₇ indicates that the benzene, pyrimidine and pyridine rings in Formula (PBM-17) are each optionally substituted with p17 R⁴⁴ groups, and each R⁴⁴ are the same or different and each independently represent halogen, C₁₋₆ alkoxy, C₁₋₆ alkyl, deuterated C₁₋₆ alkyl or halogenated C₁₋₆ alkyl;
p17 represents an integer of 0, 1, 2, 3 or 4;
one of R⁴⁵, R⁴⁶, R⁴⁷ and R⁴⁸ represents a bond, and the remaining are the same or different and each independently represent hydrogen, halogen, or hydroxy, wherein when one of R⁴⁵, R⁴⁶, R⁴⁷ and R⁴⁸ represents a bond, the benzene ring or methylene in Formula (PBM-18) connected to the bond is directly connected to R¹;
symbol “ [symbol] ” connected to double bond in Formula (PBM-18) represents a bond in stereo-configuration (cis or trans configuration);
(R⁴⁹)ₚ₁₈ indicates that 1,2,3,4-tetrahydronaphthalene ring in Formula (PBM-19) is substituted with p18 R⁴⁹ groups, and each R⁴⁹ are the same or different and each independently represent hydrogen, hydroxy, halogen, C₁₋₆ alkyl, deuterated C₁₋₆ alkyl or halogenated C₁₋₆ alkyl;
p18 represents an integer of 0, 1, 2, 3 or 4; and
one of R⁵⁰ and R⁵¹ represents a bond, and another represents hydrogen, halogen, C₁₋₆ alkyl, deuterated C₁₋₆ alkyl or halogenated C₁₋₆ alkyl, wherein when one of R⁵⁰ and R⁵¹ represents a bond, the benzene ring in Formula (PBM-19) connected to the bond is directly connected to R¹.

In some embodiments, PBM represents a small molecule ligand targeting EGFR, including but not limited to represent the structures of Formula (PBM-1-1A), Formula (PBM-1-1B), Formula (PBM-1-1C) or Formula (PBM-1-1D):

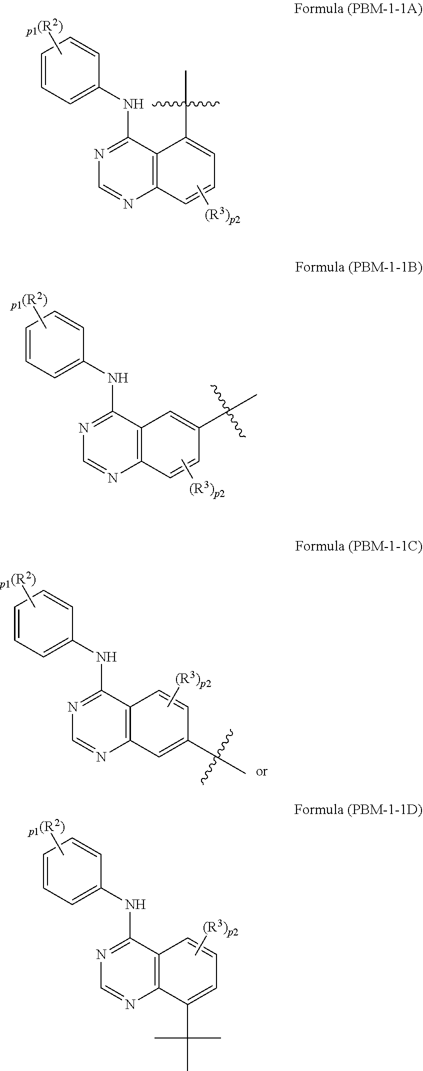

wherein (R²)ₚ₁ indicates that the benzene ring in Formula (PBM-1-1A), Formula (PBM-1-1B), Formula (PBM-1-1C) or Formula (PBM-1-1D) is substituted with p1 R², and each R² are the same or different and each independently represent halogen (e.g., fluorine, chlorine, bromine, or iodine) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—);
(R³)ₚ₂ indicates that the quinazoline ring in Formula (PBM-1-1A), Formula (PBM-1-1B), Formula (PBM-1-1C) or Formula (PBM-1-1D) is substituted with p2 R³, and each R³ are the same or different and each independently represent C₁₋₁₀ alkyl (e.g., C₁₋₈ alkyl, C₁₋₆ alkyl or C₁₋₄ alkyl, such as methyl, ethyl, propyl, isopropyl, butyl, sec-butyl, tert-butyl, pentyl, hexyl, heptyl or octyl), deuterated C₁₋₁₀ alkyl (e.g., perdeuterated C₁₋₈ alkyl, perdeuterated C₁₋₆ alkyl or perdeuterated C₁₋₄ alkyl, such as CD₃, CD₃CD₂-, CD₃CD₂CD₂- etc.), C₁₋₁₀ alkoxy (e.g., C₁₋₈ alkoxy, C₁₋₆ alkoxy or C₁₋₄ alkoxy, such as methoxy, ethoxy, propoxy, isopropoxy, butoxy, sec-butoxy or tert-butoxy), heterocyclyloxy (in which the heterocyclyl is, for example, a 4- to 20-membered monocyclic or bicyclic heterocyclyl group containing one or more, such as 1-4 or 1, 2, 3, or 4 heteroatoms selected from nitrogen, oxygen, and sulfur) or —NHC(O)R⁴, where R⁴ is C₁₋₁₀ alkyl (e.g., C₁₋₈ alkyl, C₁₋₆ alkyl or C₁₋₄ alkyl, such as methyl, ethyl, propyl, isopropyl, butyl, sec-butyl, tert-butyl, pentyl, hexyl, heptyl or octyl) or C₂₋₁₀ alkenyl (e.g., C₂₋₆ alkenyl or C₂₋₄ alkenyl, such as vinyl, propenyl, butenyl or pentenyl), e.g., R³ represents methyl, methoxy,

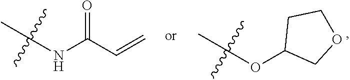

such as

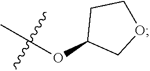 [SMILES: *O[C@H]1CCOC1]

p1 represents an integer of 1, 2, 3, 4 or 5; and
p2 represents an integer of 1, 2 or 3;

In some embodiments, R³ represents heterocyclyl-O—, and the heterocyclyl is 4- to 20-membered (e.g., 4- to 15-membered, 4- to 12-membered, 4- to 10-membered, 4- to 7-membered, or 4 to 6-membered) monocyclic or bicyclic heterocyclyl group containing one or more (e.g., 1-4 or 1, 2, 3, or 4) heteroatoms selected from nitrogen, oxygen, and sulfur, and includes but is not limited to azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, diazacycloheptanyl (e.g., 1,4-diazacycloheptan-1-yl), azacyclooctyl, diazacyclooctyl, azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl (e.g., 3,8-diazabicyclo[3.2.1]octan-3-yl), diazabicyclo[2.2.2]octanyl (e.g., 2,5-diazabicyclo[2.2.2]octan-2-yl).

In some embodiments, R³ represents:
methyl, ethyl, propyl, isopropyl, butyl, sec-butyl, tert-butyl, pentyl, hexyl, heptyl or octyl;
methoxy, ethoxy, propoxy, isopropoxy, butoxy, sec-butoxy or tert-butoxy;
—NHC(O)—C₂₋₁₀ alkenyl, e.g., —NHC(O)-vinyl, —NHC(O)-propenyl or —NHC(O)-butenyl; or

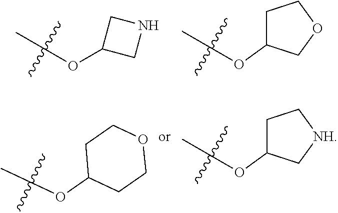

In some embodiments, PBM represents the structures of Formula (PBM-1-1), Formula (PBM-1-2), Formula (PBM-1-3) or Formula (PBM-1-4):

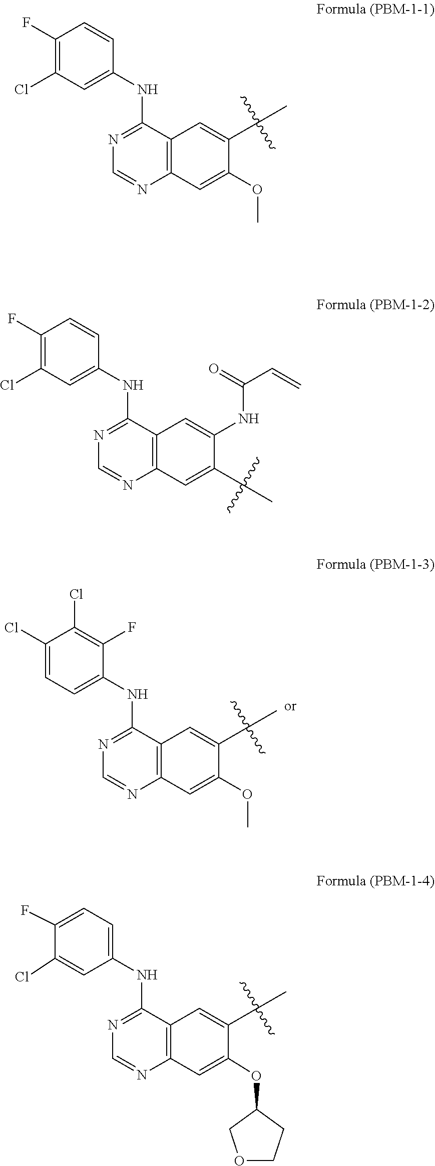 [SMILES: *c1cc2c(Nc3ccc(F)c(Cl)c3)ncnc2cc1OC ; *c1cc2ncnc(Nc3ccc(F)c(Cl)c3)c2cc1NC(=O)C=C ; *c1cc2c(Nc3ccc(Cl)c(Cl)c3F)ncnc2cc1OC ; *c1cc2c(Nc3ccc(F)c(Cl)c3)ncnc2cc1O[C@H]1CCOC1]

In some embodiments, PBM represents a small molecule ligand targeting EGFR, including but not limited to represent the structures of Formula (PBM-2-1A), Formula (PBM-2-1B), Formula (PBM-2-1C) or Formula (PBM-2-1D):

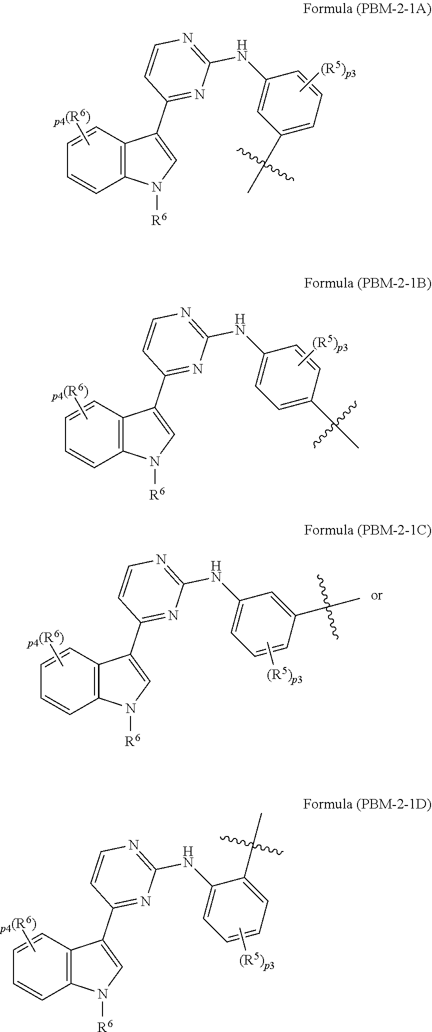

wherein (R⁵)ₚ₃ indicates that the benzene ring in Formula (PBM-2-1A), Formula (PBM-2-1B), Formula (PBM-2-1C) or Formula (PBM-2-1D) is substituted with p3 R⁵, and each R⁵ are the same or different and each independently represent C₁₋₁₀ alkyl (e.g., C₁₋₈ alkyl, C₁₋₆ alkyl or C₁₋₄ alkyl, such as methyl, ethyl, propyl, isopropyl, butyl, sec-butyl, tert-butyl, pentyl, hexyl, heptyl or octyl), deuterated C₁₋₁₀ alkyl (e.g., perdeuterated C₁₋₈ alkyl, perdeuterated C₁₋₆ alkyl or perdeuterated C₁₋₄ alkyl, such as CD₃, CD₃CD₂-, CD₃CD₂CD₂- etc.), C₁₋₁₀ alkoxy (e.g., C₁₋₈ alkoxy, C₁₋₆ alkoxy or C₁₋₄ alkoxy, such as methoxy, ethoxy, propoxy, isopropoxy, butoxy, sec-butoxy or tert-butoxy), or —NHC(O)R⁴, where R⁴ is C₁₋₁₀ alkyl (e.g., C₁₋₈ alkyl, C₁₋₆ alkyl or C₁₋₄ alkyl, such as methyl, ethyl, propyl, isopropyl, butyl, sec-butyl, tert-butyl, pentyl, hexyl, heptyl or octyl), deuterated C₁₋₁₀ alkyl (e.g., perdeuterated C₁₋₈ alkyl, perdeuterated C₁₋₆ alkyl or perdeuterated C₁₋₄ alkyl, such as CD₃, CD₃CD₂-, CD₃CD₂CD₂-etc.) or C₂₋₁₀ alkenyl (e.g., C₂₋₆ alkenyl or C₂₋₄ alkenyl, such as vinyl, propenyl, butenyl or pentenyl), e.g., R⁵ represents methyl, methoxy or

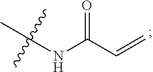 [SMILES: *NC(=O)C=C]

p3 represents an integer of 1, 2, 3 or 4;
(R⁶)ₚ₄ indicates that the 1H-indole ring in Formula (PBM-2-1A), Formula (PBM-2-1B), Formula (PBM-2-1C) or Formula (PBM-2-1D) is substituted with p4 R⁶, and each R⁶ are the same or different and each independently represent C₁₋₁₀ alkyl (e.g., C₁₋₈ alkyl, C₁₋₆ alkyl or C₁₋₄ alkyl, such as methyl, ethyl, propyl, isopropyl, butyl, sec-butyl, tert-butyl, pentyl, hexyl, heptyl or octyl) or deuterated C₁₋₁₀ alkyl (e.g., perdeuterated C₁₋₈ alkyl, perdeuterated C₁₋₆ alkyl or perdeuterated C₁₋₄ alkyl, such as CD₃, CD₃CD₂-, CD₃CD₂CD₂- etc.); and
p4 represents an integer of 0, 1, 2 or 3.

In some embodiments, PBM represents the structure of Formula (PBM-2-1):

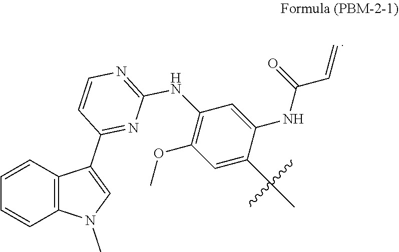 [SMILES: *c1cc(OC)c(Nc2nccc(-c3cn(C)c4ccccc34)n2)cc1NC(=O)C=C]

In some embodiments, PBM represents a small molecule ligand targeting EGFR, including but not limited to represent the structures of Formula (PBM-3-1A), Formula (PBM-3-1B), Formula (PBM-3-1C) or Formula (PBM-3-1D):

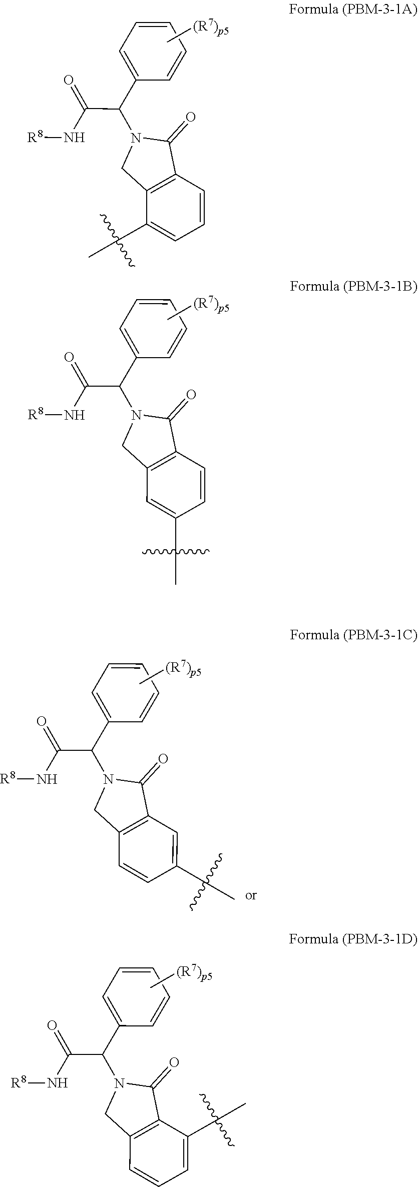

wherein (R⁷)ₚ₅ indicates that the benzene ring in Formula (PBM-3-1A), Formula (PBM-3-1B), Formula (PBM-3-1C) or Formula (PBM-3-1D) is substituted with p5 R⁷, and each R⁷ are the same or different and each independently represent halogen (e.g., fluorine, chlorine, bromine, or iodine), hydroxy, C₁₋₆ alkyl (e.g., C₁₋₈ alkyl, C₁₋₆ alkyl or C₁₋₄ alkyl, such as methyl, ethyl, propyl, isopropyl, butyl, sec-butyl, tert-butyl, pentyl, hexyl, heptyl or octyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), NO₂, NH₂, or cyano;
p5 represents an integer of 2, 3, 4 or 5; and
R⁸ represents C₆₋₁₀ aryl or heteroaryl containing one or two 5- to 7-membered rings and 1 to 4 heteroatoms selected from nitrogen, oxygen, and sulfur, wherein the C₆₋₁₀ aryl and heteroaryl are each independently optionally substituted with one or more (e.g., 1-6, such as 1, 2, 3, 4, 5 or 6) R^{4a}, and each R^{4a} are the same or different and are each independently halogen (e.g., fluorine, chlorine, bromine, or iodine), hydroxy, cyano, amino, nitro, C₁₋₆ alkyl (e.g., C₁₋₈ alkyl, C₁₋₆ alkyl or C₁₋₄ alkyl, such as methyl, ethyl, propyl, isopropyl, butyl, sec-butyl, tert-butyl, pentyl, hexyl, heptyl or octyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—).

In some embodiments, R⁸ represents C₆₋₁₀ aryl, such as but not limited to phenyl or naphthyl, each of which is optionally substituted with one or more (e.g., 1-6, such as 1, 2, 3, 4, 5 or 6) R^{4a} as defined above.

In some embodiments, R⁸ represents heteroaryl containing one or two 5- to 7-membered rings and 1 to 4 heteroatoms selected from nitrogen, oxygen, and sulfur. In a sub-embodiment, R⁸ represents 5- to 14-membered monocyclic or bicyclic aromatic ring group containing one or more (e.g., from 1 to 6, or from 1 to 5, or from 1 to 4, or from 1 to 3) heteroatoms selected from the group consisting of oxygen, nitrogen, and sulfur. In a sub-embodiment, examples of heteroaryl include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-a]pyrimidinyl, imidazo[1,2a]pyridyl, 1H-pyrrolo[3,2-b]pyridyl, 1H-pyrrolo[2,3-b]pyridyl, pyrrolo[2,1-b]thiazolyl or imidazo[2,1-b]thiazolyl. The heteroaryl group is optionally substituted with one or more (e.g., 1-6, such as 1, 2, 3, 4, 5 or 6) R^{4a} as defined above.

In some embodiments, PBM represents the structure of Formula (PBM-3-1):

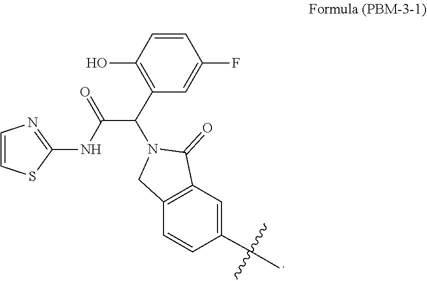 [SMILES: *c1ccc2c(c1)C(=O)N(C(C(=O)Nc1nccs1)c1cc(F)ccc1O)C2]

In some embodiments, PBM represents a small molecule ligand targeting EGFR, including but not limited to represent the structure of Formula (PBM-4):

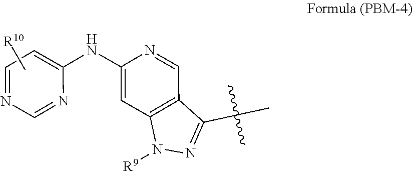

wherein R⁹ represents C₁₋₁₀ alkyl (e.g., C₁₋₈ alkyl, C₁₋₆ alkyl or C₁₋₄ alkyl, such as methyl, ethyl, propyl, isopropyl

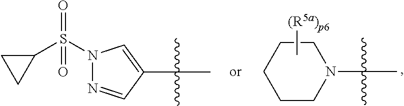

butyl, sec-butyl, tert-butyl, pentyl, hexyl, heptyl or octyl) or deuterated C₁₋₁₀ alkyl (e.g., perdeuterated C₁₋₈ alkyl, perdeuterated C₁₋₆ alkyl or perdeuterated C₁₋₄ alkyl, such as CD₃, CD₃CD₂-, CD₃CD₂CD₂- etc.); and
R¹⁰ represents

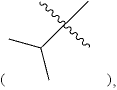 [SMILES: *C(C)C]

wherein (R^{5a})ₚ₆ indicates that the piperidine ring is substituted with p6 R^{5a}, and each R^{5a} are the same or different and each independently represent deuterium, halogen (e.g., fluorine, chlorine, bromine, or iodine), hydroxy, C₁₋₆ alkyl (e.g., C₁₋₈ alkyl, C₁₋₆ alkyl or C₁₋₄ alkyl, such as methyl, ethyl, propyl, isopropyl, butyl, sec-butyl, tert-butyl, pentyl, hexyl, heptyl or octyl), halogenated C₁₋₆ alkyl, deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), deuterated C₁₋₆ alkoxy (e.g., perdeuterated C₁₋₄ alkoxy, such as CD₃-O—, CD₃CD₂-O—, or CD₃CD₂CD₂-O—), NO₂, NH₂, or cyano, p6 represents an integer of 1, 2, 3, 4 or 5.

In some embodiments, PBM represents the structures of Formula (PBM-4-1) or Formula (PBM-4-2):

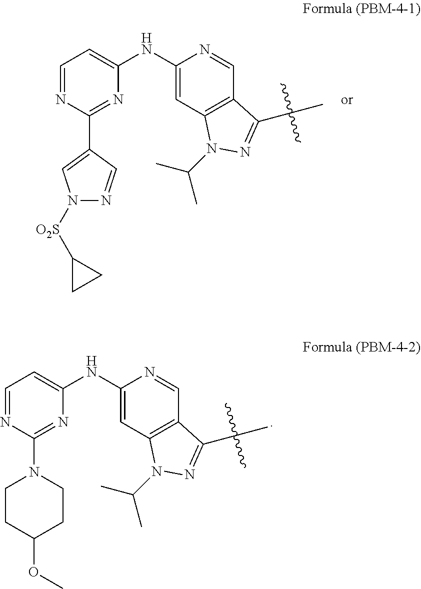 [SMILES: *c1nn(C(C)C)c2cc(Nc3ccnc(-c4cnn(S(=O)(=O)C5CC5)c4)n3)ncc12 ; *c1nn(C(C)C)c2cc(Nc3ccnc(N4CCC(OC)CC4)n3)ncc12]

In some embodiments, PBM represents a small molecule ligand targeting EGFR, including but not limited to represent the structure of Formula (PBM-5-1A), Formula (PBM-5-1B), Formula (PBM-5-1C) or Formula (PBM-5-1D):

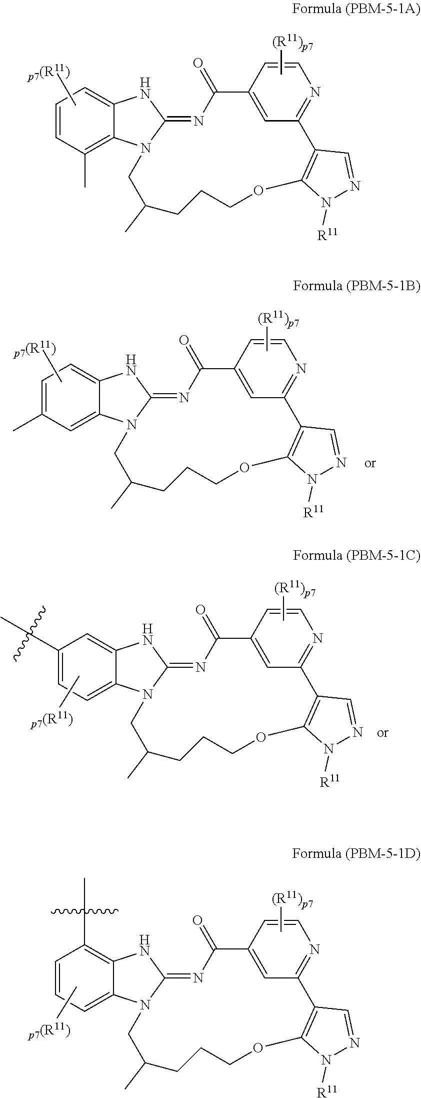

wherein (R¹¹)ₚ₇ indicates that the benzene ring and the pyridine ring in Formula (PBM-5-1A), Formula (PBM-5-1B), Formula (PBM-5-1C) or Formula (PBM-5-1D) are each optionally substituted with p7 R¹¹, and each R¹¹ are the same or different and each independently represent halogen (e.g., fluorine, chlorine, bromine, or iodine), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), or deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), p7 represents an integer of 0, 1, 2 or 3.

In some embodiments, PBM represents the structure of Formula (PBM-5-1):

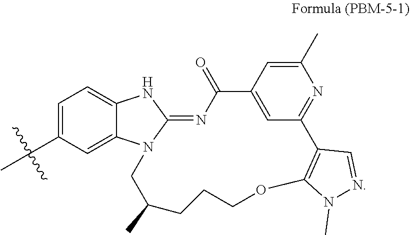 [SMILES: *c1ccc2c(c1)N1C[C@H](C)CCCOc3c(cnn3C)-c3cc(cc(C)n3)C(=O)/N=C/1N2]

In some embodiments, PBM represents a small molecule ligand targeting AR, including but not limited to represent the structure of Formula (PBM-6-1A):

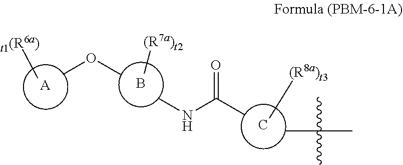

wherein ring A in Formula (PBM-6-1A) represents C₆₋₁₀ aryl or 5- to 20-membered monocyclic or bicyclic heteroaryl containing from 1 to 4 heteroatoms selected from the group consisting of oxygen, nitrogen, and sulfur, wherein the ring A is optionally substituted with t1 (e.g., 1-6, such as 1, 2, 3, 4, 5 or 6) R^{6a}, and each R^{6a} are the same or different and each independently represent deuterium, halogen (e.g., fluorine, chlorine, bromine, or iodine), hydroxy, cyano, amino, nitro, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—), C₂₋₆ alkenyl (e.g., vinyl, propenyl or butenyl), or C₂₋₆ alkynyl (e.g., ethynyl, propynyl, or butynyl);
ring B in Formula (PBM-6-1A) represents C₃₋₁₅ cycloalkyl or 3- to 20-membered heterocyclyl containing from 1 to 4 heteroatoms selected from the group consisting of oxygen, nitrogen, and sulfur, wherein the ring B is optionally substituted with t2 (e.g., 1-6, such as 1, 2, 3, 4, 5 or 6) R^{7a}, and each R₇ₐ are the same or different and each independently represent deuterium, halogen (e.g., fluorine, chlorine, bromine, or iodine), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—);
ring C in Formula (PBM-6-1A) represents 5- to 20-membered monocyclic or bicyclic heteroaryl containing from 1 to 4 heteroatoms selected from the group consisting of oxygen, nitrogen, and sulfur, wherein the ring C is optionally substituted with t3 (e.g., 1-6, such as 1, 2, 3, 4, 5 or 6) R^{8a}, and each R₈ₐ are the same or different and each independently represent deuterium, halogen (e.g., fluorine, chlorine, bromine, or iodine), hydroxy, cyano, amino, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—);
t1 represents an integer of 1, 2, 3, 4, 5 or 6;
t2 represents an integer of 1, 2, 3, 4, 5 or 6; and
t3 represents an integer of 1, 2, 3, 4, 5 or 6.

In some embodiments, PBM represents the structure of Formula (PBM-6-1A-1):

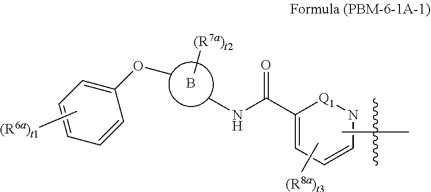

wherein (R^{6a})ₜ₁ indicates that the benzene ring in Formula (PBM-6-1A-1) is optionally substituted with t1 R^{6a}, and each R^{6a} are the same or different and each independently represent deuterium, halogen (e.g., fluorine, chlorine, bromine, or iodine), hydroxy, cyano, amino, nitro, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—), C₂₋₆ alkenyl (e.g., vinyl, propenyl or butenyl), or C₂₋₆ alkynyl (e.g., ethynyl, propynyl, or butynyl);
ring B in Formula (PBM-6-1A-1) represents C₃₋₁₅ cycloalkyl or 3- to 20-membered heterocyclyl containing from 1 to 4 heteroatoms selected from the group consisting of oxygen, nitrogen, and sulfur, wherein the ring B is optionally substituted with t2 R^{7a}, and each R^{7a} are the same or different and each independently represent deuterium, halogen (e.g., fluorine, chlorine, bromine, or iodine), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—);
Q₁ represents N or CH; and
(R^{8a})ₜ₃ indicates that the nitrogen-containing heteroaryl in Formula (PBM-6-1A-1) is optionally substituted with t3 R^{8a}, and each R^{8a} are the same or different and each independently represent deuterium, halogen (e.g., fluorine, chlorine, bromine, or iodine), hydroxy, cyano, amino, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—);
t1 represents an integer of 1, 2, 3, 4, 5 or 6;
t2 represents an integer of 1, 2, 3, 4, 5 or 6; and
t3 represents an integer of 1 or 2.

In some embodiments, Q₁ represents N. In some embodiments, Q₁ represents CH.

In some embodiments, PBM represents the structures of Formula (PBM-6-1), Formula (PBM-6-2) or Formula (PBM-6-3):

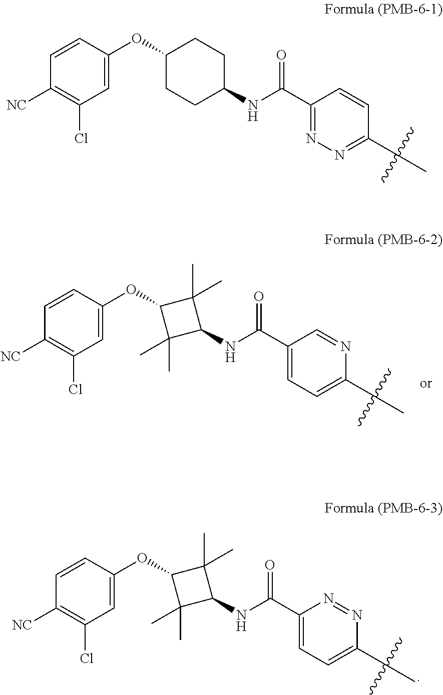 [SMILES: *c1ccc(C(=O)N[C@H]2CC[C@H](Oc3ccc(C#N)c(Cl)c3)CC2)nn1 ; *c1ccc(C(=O)N[C@H]2C(C)(C)[C@H](Oc3ccc(C#N)c(Cl)c3)C2(C)C)cn1 ; *c1ccc(C(=O)N[C@H]2C(C)(C)[C@H](Oc3ccc(C#N)c(Cl)c3)C2(C)C)nn1]

In some embodiments, PBM represents a small molecule ligand targeting BTK, including but not limited to represent the structures of Formula (PBM-7-1A), Formula (PBM-7-1B) or Formula (PBM-7-1C):

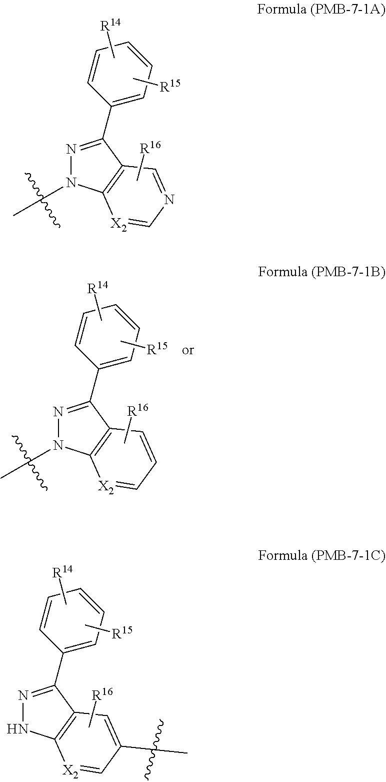

wherein X₂ represents CH or N;
R¹⁴ represents —O-aryl, —O-heteroaryl, —C₁₋₃-alkylene-NHC(O)-heteroaryl, or —C₁₋₃-alkylene-C(O)NH-heteroaryl, wherein the aryl and heteroaryl are each independently optionally substituted with one or more (e.g., 1-5, such as 1, 2, 3, 4 or 5) R^{9a}, and each R^{9a} are the same or different and each independently represent deuterium, halogen (e.g., fluorine, chlorine, bromine, or iodine), hydroxy, cyano, amino, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—);
R¹⁵ represents deuterium, hydrogen, halogen (e.g., fluorine, chlorine, bromine, or iodine), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or —NHC(O)R¹⁷, wherein R¹⁷ is C₁₋₁₀ alkyl (e.g., C₁₋₈ alkyl, C₁₋₆ alkyl or C₁₋₄ alkyl, such as methyl, ethyl, propyl, isopropyl, butyl, sec-butyl, tert-butyl, pentyl, hexyl, heptyl or octyl) or C₂₋₁₀ alkenyl (e.g., C₂₋₆ alkenyl or C₂₋₄ alkenyl, such as vinyl, propenyl, butenyl or pentenyl), e.g., R¹⁵ represents hydrogen, methyl, or

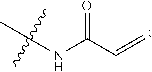 [SMILES: *NC(=O)C=C]

and
R¹⁶ represents hydrogen, cyano, or amino.

In some embodiments, R¹⁴ represents —O-aryl, —O-heteroaryl, —C₁₋₃-alkylene-NHC(O)-heteroaryl, or —C₁₋₃-alkylene-C(O)NH-heteroaryl, wherein the aryl can be optionally substituted C₆₋₁₀ aryl, such as but not limited to phenyl or naphthyl, and the heteroaryl can be optionally substituted 5 to 14-membered monocyclic or bicyclic aromatic ring group containing one or more (e.g., from 1 to 6, or from 1 to 5, or from 1 to 4, or from 1 to 3) heteroatoms selected from the group consisting of oxygen, nitrogen, and sulfur. In a sub-embodiment, R¹⁴ represents —O-phenyl or —O-naphthyl, where the phenyl or naphthyl are optionally substituted with one or more (e.g., 1-5, such as 1, 2, 3, 4 or 5) R^{9a}, and each R^{9a} are the same or different and each independently represent deuterium, halogen (e.g., fluorine, chlorine, bromine, or iodine), hydroxy, cyano, amino, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—). In a sub-embodiment, R¹⁴ represents —O-heteroaryl, —C₁₋₃-alkylene-NHC(O)-heteroaryl, or —C₁₋₃-alkylene-C(O)NH-heteroaryl, wherein the heteroaryl is optionally substituted with one or more (e.g., 1-5, such as 1, 2, 3, 4 or 5) R^{9a}, and each R^{9a} are the same or different and each independently represent deuterium, halogen (e.g., fluorine, chlorine, bromine, or iodine), hydroxy, cyano, amino, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl, or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—).

In some embodiments, PBM represents the structures of Formula (PBM-7-1) or Formula (PBM-7-2):

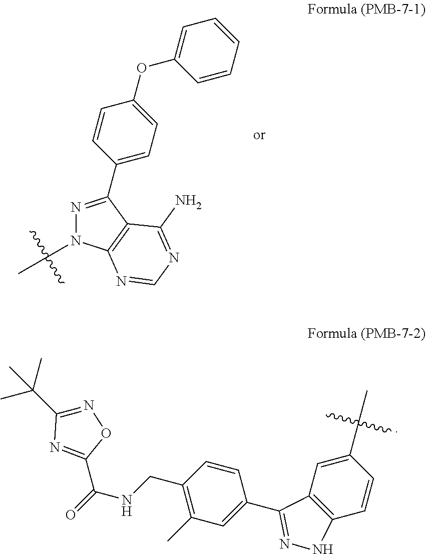 [SMILES: *n1nc(-c2ccc(Oc3ccccc3)cc2)c2c(N)ncnc21 ; *c1ccc2[nH]nc(-c3ccc(CNC(=O)c4nc(C(C)(C)C)no4)c(C)c3)c2c1]

In some embodiments, PBM represents a small molecule ligand targeting CDK4/6, including but not limited to represent the structures of Formula (PBM-8-1A), Formula (PBM-8-1B), Formula (PBM-8-1C) or Formula (PBM-8-1D):

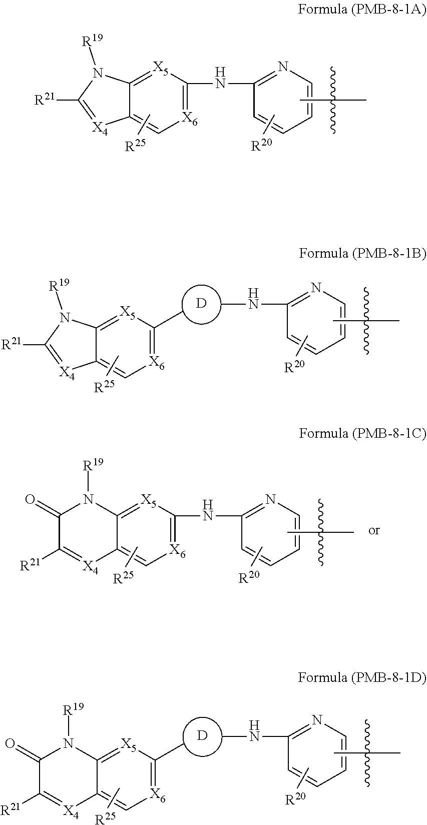

wherein R²¹ represents deuterium, hydrogen, halogen (e.g., fluorine, chlorine, bromine, or iodine), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), —C(O)—C₁₋₆ alkyl, —C(O)-deuterated C₁₋₆ alkyl, or —C(O)NR²²R²³, wherein R²² and R²³ are the same or different and each independently represent hydrogen, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl) or deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), and R²² and R²³ are not simultaneously hydrogen;
X₄ represents N or CR²⁴, wherein R²⁴ represents hydrogen, deuterium, halogen (e.g., fluorine, chlorine, bromine, or iodine), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—);
X₅ and X₆ each independently represent CH or N;
ring D represents optionally substituted heteroarylene (e.g., halogenated heteroarylene);
R¹⁹ represents hydrogen, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), or optionally substituted C₃₋₇ cycloalkyl (e.g., cyclopropyl, cyclobutyl, cyclopentyl, cyclopentenyl, cyclohexyl, cyclohexenyl, or cycloheptyl, which are optionally substituted with a substituent selected from the group consisting of halogen, C₁₋₆ alkyl, halogenated C₁₋₆ alkyl or deuterated C₁₋₆ alkyl); and
R²⁰ and R²⁵ are the same or different and each independently represent hydrogen, halogen (e.g., fluorine, chlorine, bromine, or iodine), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—).

In some embodiments, ring D represents optionally substituted heteroarylene, e.g., optionally substituted 5- to 20-membered monocyclic or bicyclic heteroarylene containing one or more (e.g., from 1 to 6, or from 1 to 5, or from 1 to 4, or from 1 to 3) heteroatoms independently selected from the group consisting of oxygen, nitrogen, and sulfur. Examples of heteroarylene include, but are not limited to, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, 4H-fluoro[3,2-b]pyrrolylene, pyrrolo[2,1-b]thiazolylene, and imidazo[2,1-b]thiazolylene. The heteroarylene is optionally substituted with one or more (e.g., 1-5, such as 1, 2, 3, 4 or 5) substituents selected from the group consisting of halogen (e.g., fluorine, chlorine, bromine, or iodine), hydroxy, cyano, amino, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—).

In some embodiments, PBM represents the structures of Formula (PBM-8-1), Formula (PBM-8-2) or Formula (PBM-8-3):

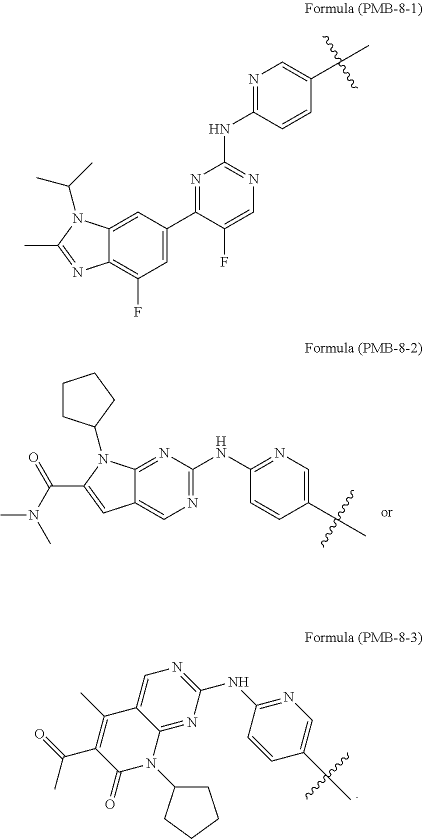 [SMILES: *c1ccc(Nc2ncc(F)c(-c3cc(F)c4nc(C)n(C(C)C)c4c3)n2)nc1 ; *c1ccc(Nc2ncc3cc(C(=O)N(C)C)n(C4CCCC4)c3n2)nc1 ; *c1ccc(Nc2ncc3c(C)c(C(C)=O)c(=O)n(C4CCCC4)c3n2)nc1]

In some embodiments, PBM represents a small molecule ligand targeting ALK, including but not limited to represent the structures of Formula (PBM-9-1A), Formula (PBM-9-1B), Formula (PBM-9-1C) or Formula (PBM-9-1D):

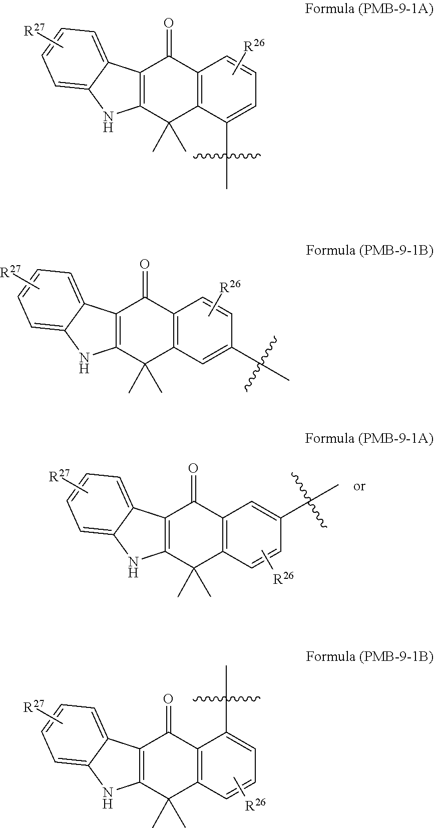

wherein R²⁶ represents hydrogen, halogen (e.g., fluorine, chlorine, bromine, or iodine), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), or deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.); and
R²⁷ represents hydrogen, cyano, halogen (e.g., fluorine, chlorine, bromine, or iodine), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), or deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.).

In some embodiments, PBM represents the structure of Formula (PBM-9-1):

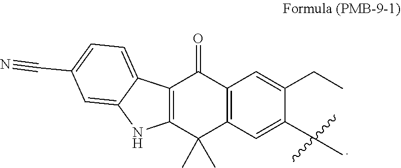 [SMILES: *c1cc2c(cc1CC)C(=O)c1c([nH]c3cc(C#N)ccc13)C2(C)C]

In some embodiments, PBM represents a small molecule ligand targeting ALK, including but not limited to represent the structure of Formula (PBM-10):

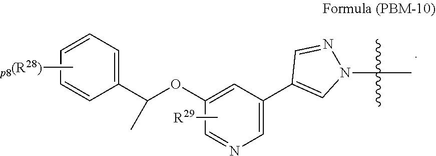

wherein (R²⁸)ₚ₈ indicates that the benzene ring in Formula (PBM-10) is substituted with p8 R²⁸ substituents, and each R²⁸ are the same or different and each independently represent halogen (e.g., fluorine, chlorine, bromine, or iodine), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), or deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.);
p8 represents an integer of 1, 2, 3 or 4; or
R²⁹ represents NR³⁰R³¹, wherein R³⁰, R³¹ are the same or different and each independently represent hydrogen, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), or deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.).

In some embodiments, PBM represents the structure of Formula (PBM-10-1):

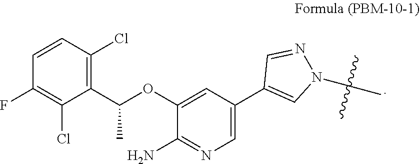 [SMILES: *n1cc(-c2cnc(N)c(O[C@H](C)c3c(Cl)ccc(F)c3Cl)c2)cn1]

In some embodiments, PBM represents the structures of Formula (PBM-11-1A), Formula (PBM-11-1B) or Formula (PBM-11-1C):

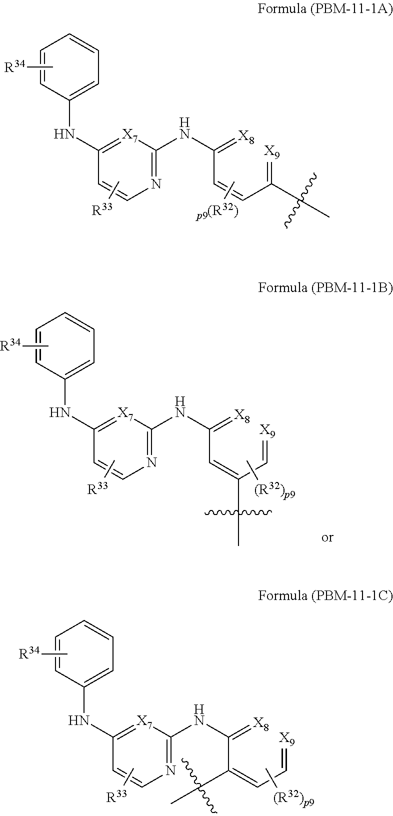

wherein X₇, X₈ and X₉ are the same or different and each independently represent N or CH;
(R³²)ₚ₉ indicates that the 6-membered ring containing X₈ and X₉ is optionally substituted with p9 R³² groups, and each R³² are the same or different and each independently represent halogen (e.g., fluorine, chlorine, bromine, or iodine), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—);
p9 represents an integer of 0, 1, 2, 3 or 4;
R³³ represents halogen (e.g., fluorine, chlorine, bromine, or iodine), C₁₋₆ alkoxy, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—); and
R³⁴ represents —C(O)NHR³⁵, —NHC(O)—R³⁵, —S(O)₂NHR³⁵, —S(O)₂R³⁵ or —P(O)(R³⁵)₂, wherein each R³⁵ are the same or different and each independently represent C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl) or deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.).

In some embodiments, PBM represents a small molecule ligand targeting ALK, including but not limited to represent the structures of Formula (PBM-11-1D), Formula (PBM-11-1E) or Formula (PBM-11-1F):

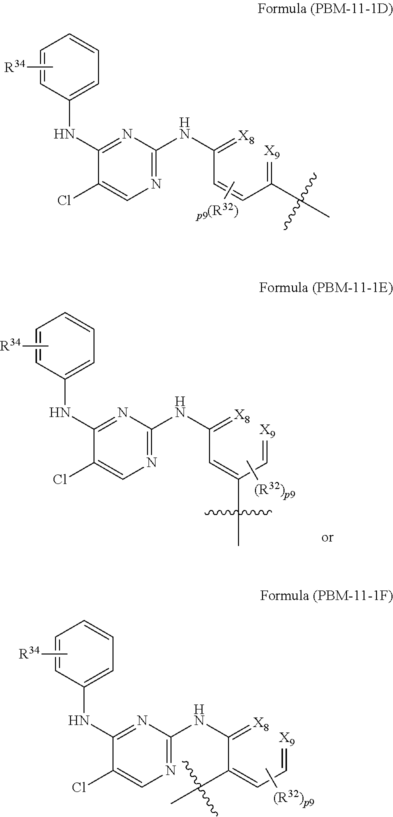

wherein X₈ and X₉ are the same or different and each independently represent N or CH;
(R³²)ₚ₉ indicates that the 6-membered ring containing X₈ and X₉ is optionally substituted with p9 R³² groups, and each R³² are the same or different and each independently represent halogen (e.g., fluorine, chlorine, bromine, or iodine), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—);
p9 represents an integer of 0, 1, 2, 3 or 4; and
R³⁴ represents —C(O)NHR³⁵, —NHC(O)—R³⁵, —S(O)₂NHR³⁵, —S(O)₂R³⁵ or —P(O)(R³⁵)₂, wherein each R³⁵ are the same or different and each independently represent C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl) or deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.).

In some embodiments, PBM represents a small molecule ligand targeting ALK, including but not limited to represent the structures of Formula (PBM-11-1) or Formula (PBM-11-2):

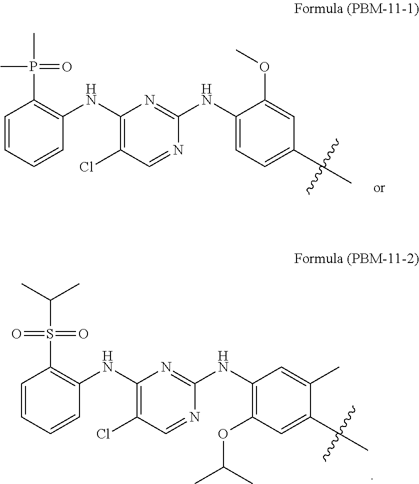 [SMILES: *c1ccc(Nc2ncc(Cl)c(Nc3ccccc3P(C)(C)=O)n2)c(OC)c1 ; *c1cc(OC(C)C)c(Nc2ncc(Cl)c(Nc3ccccc3S(=O)(=O)C(C)C)n2)cc1C]

In some embodiments, PBM represents a small molecule ligand targeting FAK, including but not limited to represent the structures of Formula (PBM-11-1G), Formula (PBM-11-1H) or Formula (PBM-11-1I):

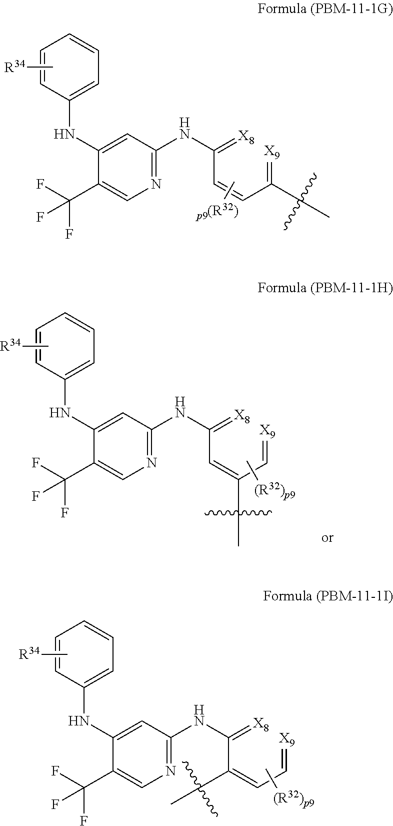

wherein X₈ and X₉ are the same or different and each independently represent N or CH;
(R³²)ₚ₉ indicates that the 6-membered ring containing X₈ and X₉ is optionally substituted with p9 R³² groups, and each R³² are the same or different and each independently represent halogen (e.g., fluorine, chlorine, bromine, or iodine), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—);
p9 represents an integer of 0, 1, 2, 3 or 4; and
R³⁴ represents —C(O)NHR³⁵, —NHC(O)—R³⁵, —S(O)₂NHR³⁵, —S(O)₂R³¹ or —P(O)(R³⁵)₂, wherein each R³⁵ are the same or different and each independently represent C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl) or deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.).

In some embodiments, PBM represents a small molecule ligand targeting FAK, including but not limited to represent the structures of Formula (PBM-11-3):

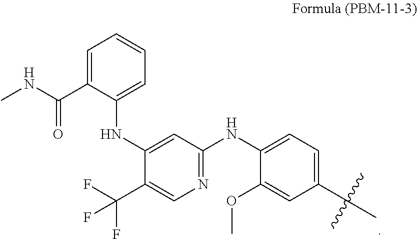 [SMILES: *c1ccc(Nc2cc(Nc3ccccc3C(=O)NC)c(C(F)(F)F)cn2)c(OC)c1]

In some embodiments, PBM represents a small molecule ligand targeting RET, including but not limited to represent the structures of Formula (PBM-11-1J), Formula (PBM-11-1K) or Formula (PBM-11-1L):

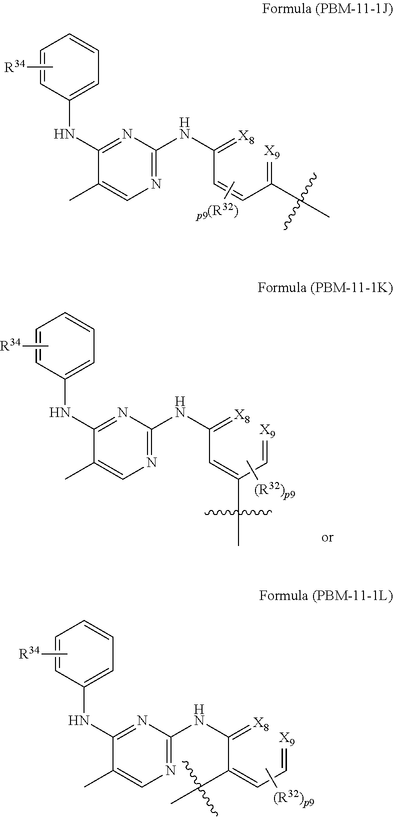

wherein X₈ and X₉ are the same or different and each independently represent N or CH;
(R³²)ₚ₉ indicates that the 6-membered ring containing X₈ and X₉ is optionally substituted with p9 R₃₂ groups, and each R³² are the same or different and each independently represent halogen (e.g., fluorine, chlorine, bromine, or iodine), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—);
p9 represents an integer of 0, 1, 2, 3 or 4; and
R³⁴ represents —C(O)NHR³⁵, —NHC(O)—R³⁵, —S(O)₂NHR³⁵, —S(O)₂R³⁵ or —P(O)(R³⁵)₂, wherein each R³⁵ are the same or different and each independently represent C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl) or deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.).

In some embodiments, PBM represents a small molecule ligand targeting RET, including but not limited to represent the structures of Formula (PBM-11-4) or Formula (PBM-11-5):

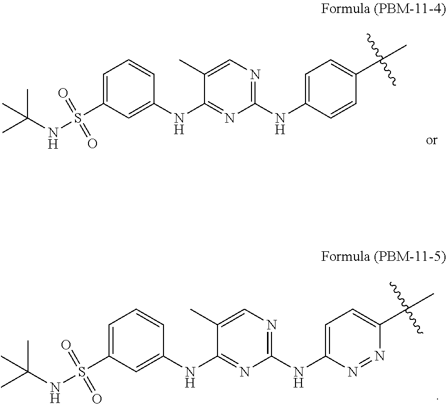 [SMILES: *c1ccc(Nc2ncc(C)c(Nc3cccc(S(=O)(=O)NC(C)(C)C)c3)n2)cc1 ; *c1ccc(Nc2ncc(C)c(Nc3cccc(S(=O)(=O)NC(C)(C)C)c3)n2)nn1]

In some embodiments, PBM represents a small molecule ligand targeting RET, including but not limited to represent the structures of Formula (PBM-12-1A), Formula (PBM-12-1B), Formula (PBM-12-1C), Formula (PBM-12-1D) or Formula (PBM-12-1E):

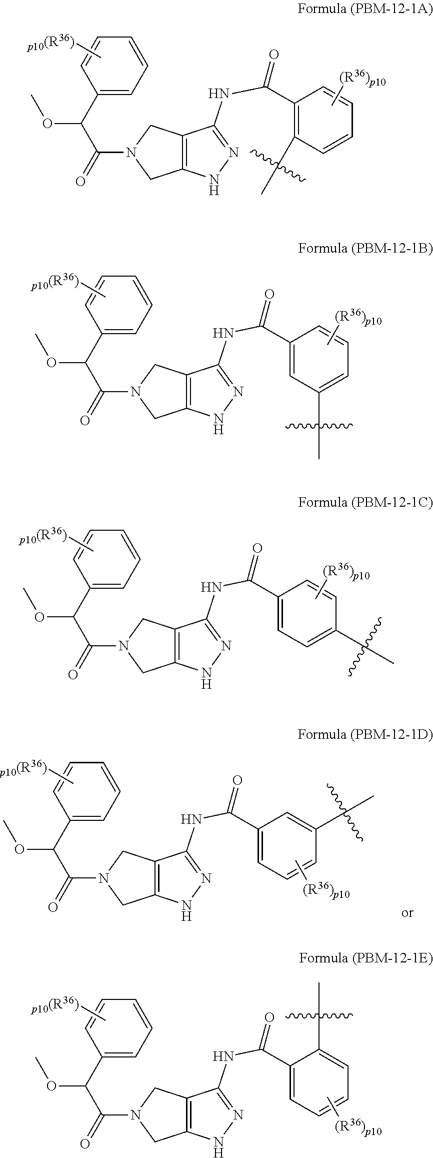

wherein (R³⁶)ₚ₁₀ indicates that the benzene ring in Formula (PBM-12-1A), Formula (PBM-12-1B), Formula (PBM-12-1C), Formula (PBM-12-1D) or Formula (PBM-12-1E) is optionally substituted with p10 R³⁶ groups, and each R³⁶ are the same or different and each independently represent halogen (e.g., fluorine, chlorine, bromine, or iodine), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—); and
p10 represents an integer of 0, 1, 2, 3 or 4.

In some embodiments, PBM represents a small molecule ligand targeting RET, including but not limited to represent the structure of Formula (PBM-12-1):

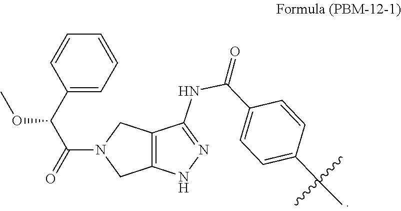 [SMILES: *c1ccc(C(=O)Nc2n[nH]c3c2CN(C(=O)[C@H](OC)c2ccccc2)C3)cc1]

In some embodiments, PBM represents a small molecule ligand targeting BET, including but not limited to represent the structure of Formula (PBM-13):

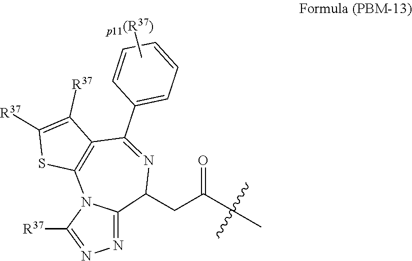

wherein (R³⁷)ₚ₁₁ indicates that the benzene ring in Formula (PBM-13) is substituted with p11 R³⁷ groups, and each R³⁷ are the same or different and each independently represent halogen (e.g., fluorine, chlorine, bromine, or iodine), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—); and
p11 represents an integer of 0, 1, 2, 3 or 4.

In some embodiments, PBM represents a small molecule ligand targeting BET, including but not limited to represent the structure of Formula (PBM-13-1):

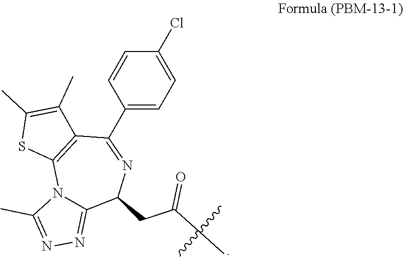 [SMILES: *C(=O)C[C@@H]1N=C(c2ccc(Cl)cc2)c2c(sc(C)c2C)-n2c(C)nnc21]

In some embodiments, PBM represents a small molecule ligand targeting BET, including but not limited to represent the structures of Formula (PBM-14-1A), Formula (PBM-14-1B), Formula (PBM-14-1C), Formula (PBM-14-1D) or Formula (PBM-14-1E):

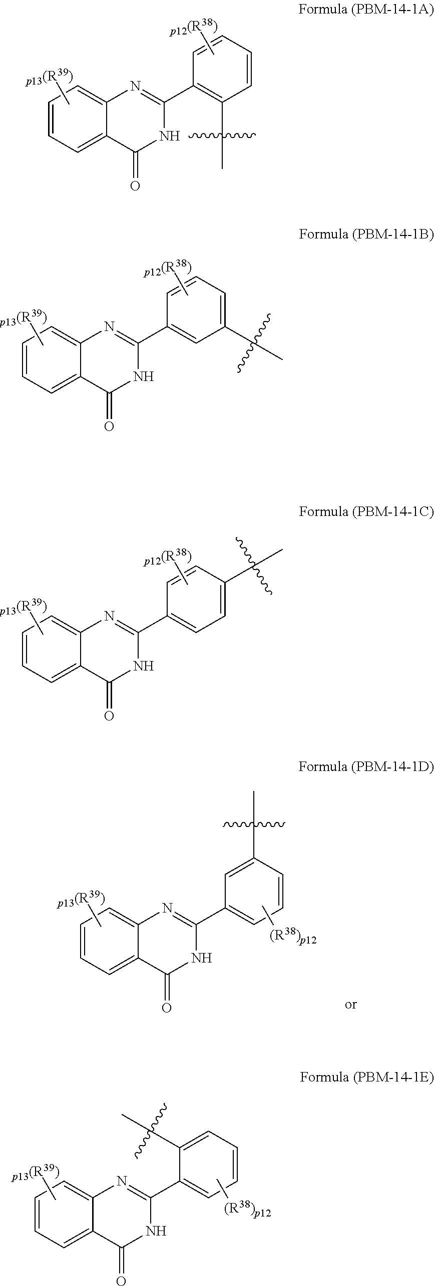

wherein (R³⁸)ₚ₁₂ indicates that the benzene ring in Formula (PBM-14-1A), Formula (PBM-14-1B), Formula (PBM-14-1C), Formula (PBM-14-1D) or Formula (PBM-14-1E) is substituted with p12 R³⁸ groups, and each R³⁸ are the same or different and each independently represent halogen (e.g., fluorine, chlorine, bromine, or iodine), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—);
p12 represents an integer of 0, 1, 2, 3 or 4;
(R³⁹)ₚ₁₃ indicates that the 4-oxo-3,4-dihydroquinazoline ring in Formula (PBM-14-1A), Formula (PBM-14-1B), Formula (PBM-14-1C), Formula (PBM-14-1D) or Formula (PBM-14-1E) is substituted with p13 R³⁹ groups, and each R³⁹ are the same or different and each independently represent halogen (e.g., fluorine, chlorine, bromine, or iodine), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—); and
p13 represents an integer of 1, 2, 3 or 4.

In some embodiments, PBM represents a small molecule ligand targeting BET, including but not limited to represent the structure of Formula (PBM-14-1):

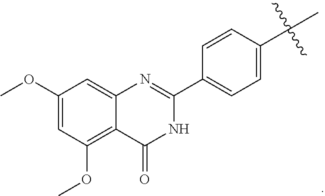 [SMILES: *c1ccc(-c2nc3cc(OC)cc(OC)c3c(=O)[nH]2)cc1]

In some embodiments, PBM represents a small molecule ligand targeting BCR-ABL, including but not limited to represent the structures of Formula (PBM-15-1A) or Formula (PBM-15-1B):

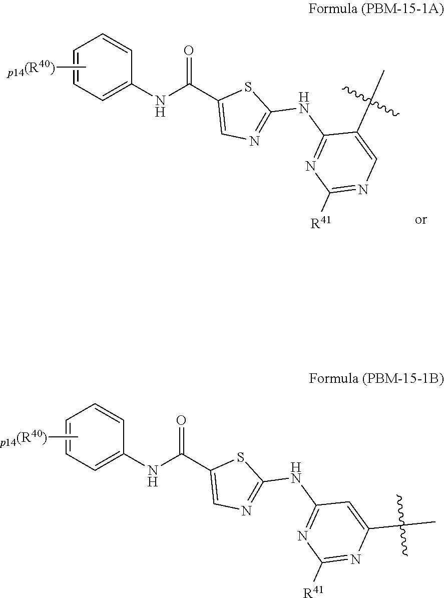

wherein (R⁴⁰)ₚ₁₄ indicates that the benzene ring in Formula (PBM-15-1A) or Formula (PBM-15-1B) is substituted with p14 R⁴⁰ groups, and each R⁴⁰ are the same or different and each independently represent halogen (e.g., fluorine, chlorine, bromine, or iodine), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—);
p14 represents an integer of 1, 2, 3 or 4; and
R⁴¹ represents halogen (e.g., fluorine, chlorine, bromine, or iodine), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—).

In some embodiments, PBM represents a small molecule ligand targeting BCR-ABL, including but not limited to represent the structure of Formula (PBM-15-1):

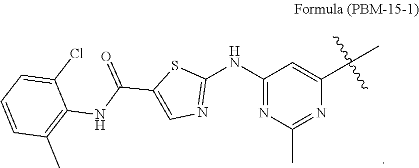 [SMILES: *c1cc(Nc2ncc(C(=O)Nc3c(C)cccc3Cl)s2)nc(C)n1]

In some embodiments, PBM represents a small molecule ligand targeting BCR-ABL, including but not limited to represent the structures of Formula (PBM-16-1A), Formula (PBM-16-1B), Formula PBM-16-1C) or Formula (PBM-16-1D):

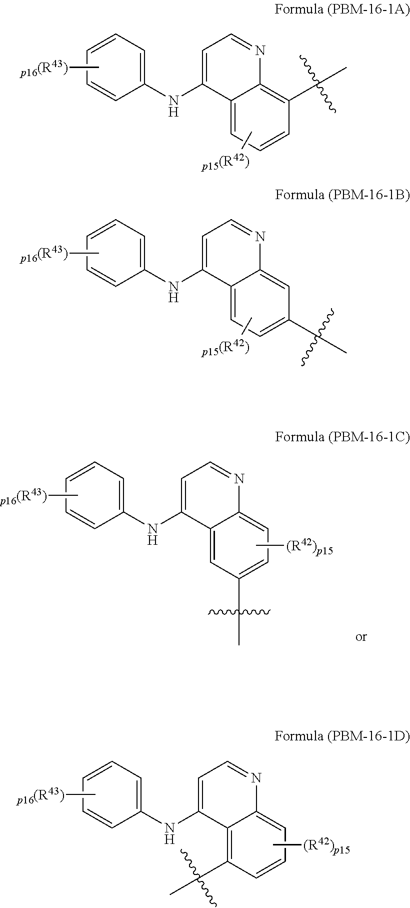

wherein (R⁴²)ₚ₁₅ indicates that the quinoline ring in Formula (PBM-16-1A), Formula (PBM-16-1B), Formula (PBM-16-1C) or Formula (PBM-16-1D) is substituted with p15 R⁴² groups, and each R⁴² are the same or different and each independently represent halogen (e.g., fluorine, chlorine, bromine, or iodine), cyano, C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—);
p15 represents an integer of 2, 3 or 4;
(R⁴³)ₚ₁₆ indicates that the benzene ring in Formula (PBM-16-1A), Formula (PBM-16-1B), Formula (PBM-16-1C) or Formula (PBM-16-1D) is substituted with p16 R⁴³ groups, and each R⁴³ are the same or different and each independently represent halogen (e.g., fluorine, chlorine, bromine, or iodine), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—); and
p16 represents an integer of 3 or 4.

In some embodiments, PBM represents a small molecule ligand targeting BCR-ABL, including but not limited to represent the structure of Formula (PBM-16-1):

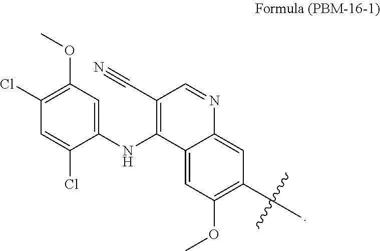 [SMILES: *c1cc2ncc(C#N)c(Nc3cc(OC)c(Cl)cc3Cl)c2cc1OC]

In some embodiments, PBM represents a small molecule ligand targeting BCR-ABL, including but not limited to represent the structures of Formula (PBM-17-1A), Formula (PBM-17-1B), Formula (PBM-17-1C), Formula (PBM-17-1D) or Formula (PBM-17-1E):

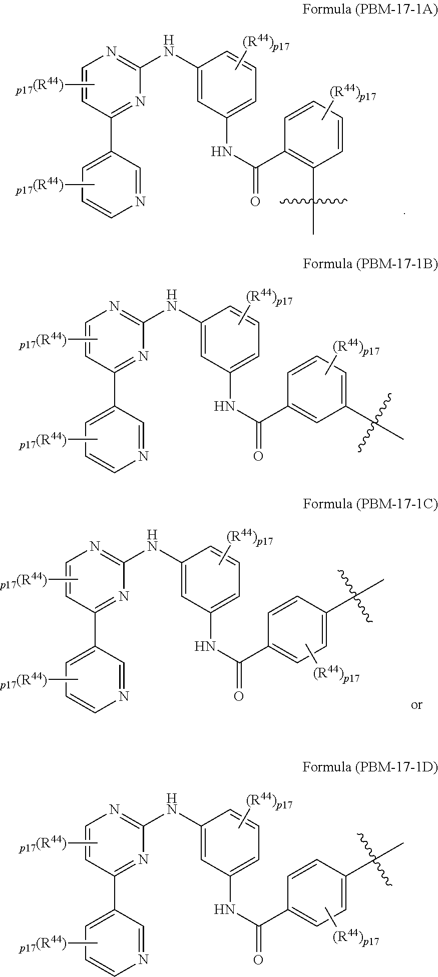

wherein (R₄)ₚ₁₇ indicates that the benzene, pyrimidine and pyridine rings in Formula (PBM-17-1A), Formula (PBM-17-1B), Formula (PBM-17-1C), Formula (PBM-17-1D) or Formula (PBM-17-1E) are each optionally substituted with p17 R⁴⁴ groups, and each R⁴⁴ are the same or different and each independently represent halogen (e.g., fluorine, chlorine, bromine, or iodine), C₁₋₆ alkoxy (e.g., C₁₋₄ alkoxy, such as methoxy, ethoxy, propoxy, isopropoxy, butoxy, sec-butoxy or tert-butoxy), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—); and
p17 represents an integer of 0, 1, 2, 3 or 4.

In some embodiments, PBM represents a small molecule ligand targeting BCR-ABL, including but not limited to represent the structure of Formula (PBM-17-1):

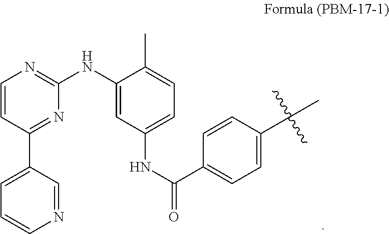 [SMILES: *c1ccc(C(=O)Nc2ccc(C)c(Nc3nccc(-c4cccnc4)n3)c2)cc1]

In some embodiments, PBM represents a small molecule ligand targeting ER, including but not limited to represent the structures of Formula (PBM-18-1A), Formula (PBM-18-1B), Formula (PBM-18-1C) or Formula (PBM-18-1D):

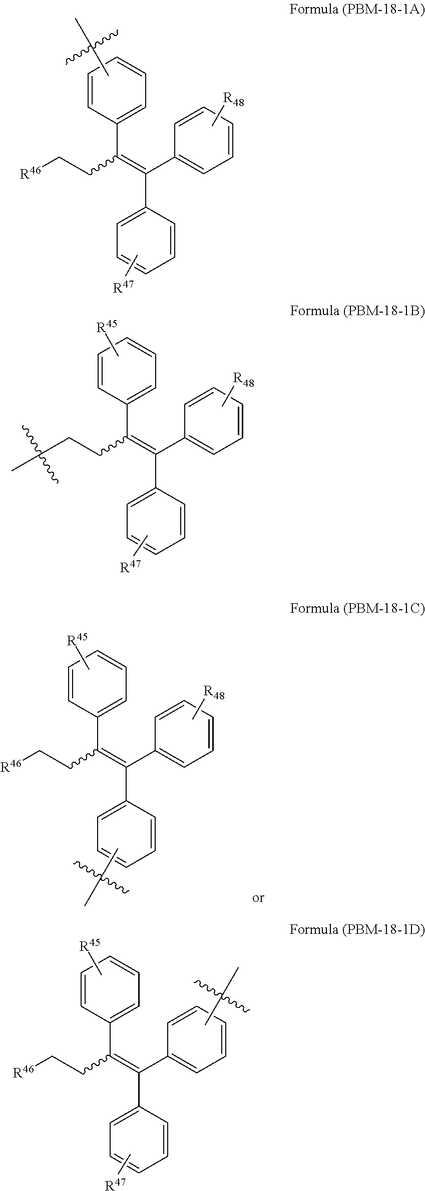

wherein R⁴⁵, R⁴⁶, R⁴⁷ and R⁴⁸ are the same or different and each independently represent hydrogen, halogen (e.g., fluorine, chlorine, bromine, or iodine), or hydroxy; and
symbol “ [symbol] ” connected to the double bond in Formula (PBM-18-1A), Formula (PBM-18-1B), Formula (PBM-18-1C) or Formula (PBM-18-1D) represents a bond in stereo-configuration (cis or trans configuration).

In some embodiments, PBM represents a small molecule ligand targeting ER, including but not limited to represent the structures of Formula (PBM-18-1), Formula (PBM-18-2), Formula (PBM-18-3), Formula (PBM-18-4), Formula (PBM-18-5) or Formula (PBM-18-6):

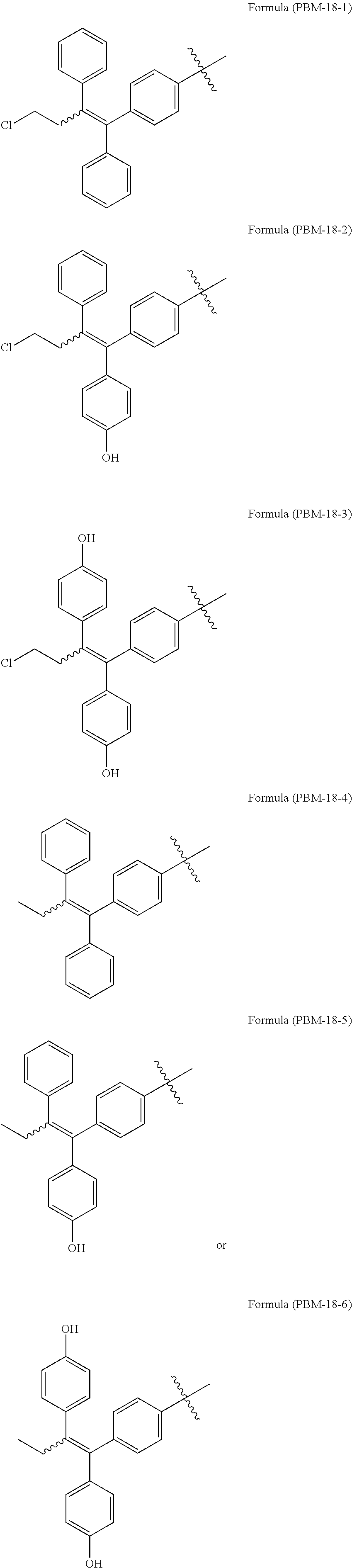 [SMILES: *c1ccc(C(=C(CCCl)c2ccccc2)c2ccccc2)cc1 ; *c1ccc(C(=C(CCCl)c2ccccc2)c2ccc(O)cc2)cc1 ; *c1ccc(C(=C(CCCl)c2ccc(O)cc2)c2ccc(O)cc2)cc1 ; *c1ccc(C(=C(CC)c2ccccc2)c2ccccc2)cc1 ; *c1ccc(C(=C(CC)c2ccccc2)c2ccc(O)cc2)cc1 ; *c1ccc(C(=C(CC)c2ccc(O)cc2)c2ccc(O)cc2)cc1]

In some embodiments, PBM represents a small molecule ligand targeting ER, including but not limited to represent the structures of Formula (PBM-19-1A) or Formula (PBM-19-1B):

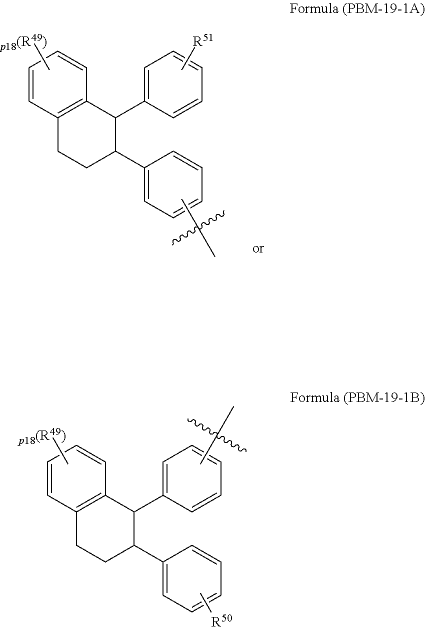

wherein (R⁴⁹)ₚ₁₈ indicates that 1,2,3,4-tetrahydronaphthalene ring in Formula (PBM-19-1A) or Formula (PBM-19-1B) is substituted with p18 R⁴⁹ groups, and each R⁴⁹ are the same or different and each independently represent hydrogen, hydroxy, halogen (e.g., fluorine, chlorine, bromine, or iodine), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—);
p18 represents an integer of 0, 1, 2, 3 or 4; and
R⁵⁰ and R⁵¹ each independently hydrogen, halogen (e.g., fluorine, chlorine, bromine, or iodine), C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.) or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—).

In some embodiments, PBM represents a small molecule ligand targeting ER, including but not limited to represent the structure of Formula (PBM-19-1):

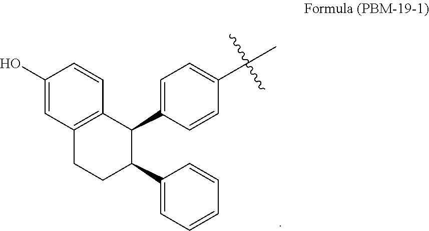 [SMILES: *c1ccc([C@@H]2c3ccc(O)cc3CC[C@@H]2c2ccccc2)cc1]

In some embodiments, R¹ represents the structures of the following formula:
a bond, —O—, C₁₋₆ alkylene (e.g., methylene, ethylene, propylene, isopropylene, butylene, tert-butylene, pentylene and hexylene) optionally substituted with a substituent selected from halogen and C₁₋₃ alkyl, —N(R^{1a})—R^{2a}—*** (e.g., —N(CH₃)—CH₂CH₂—***, —NH—CH₂CH₂—***), —O—R^{2a}—*** (e.g., —O—(CH₂)₃—***, —O—CH₂—*** and —O—(CH₂)₂—***), —C(O)NH—R^{2a}—*** (e.g., —C(O)NH—(CH₂)₂—***, —C(O)NH—(CH₂)₃—***, —C(O)NH—CH═CH—CH₂—***, and —C(O)NH—CH₂—CH═CH—CH₂—***,), or —NHC(O)—R^{2a}—*** (e.g., —NHC(O)—CH═CH—CH₂—**, —NHC(O)—CH₂═CH—CH₂***, —NHC(O)—(CH₂)₂—***, and —NHC(O)—(CH₂)₃—***), wherein symbol *** indicates the point of attachment to W¹, R^{1a} is hydrogen or C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), and R^{2a} is C₁₋₆ alkylene (e.g., methylene, ethylene, propylene, isopropylene, butylene, tert-butylene, pentylene and hexylene) optionally substituted with a substituent selected from halogen and C₁₋₃ alkyl, or C₂₋₆ alkenylene (e.g., vinylene, propenylene, allylidene, butenylene, pentenylene, and hexenylene) optionally substituted with a substituent selected from halogen and C₁₋₃ alkyl; or
phenylene or naphthylene, wherein the phenylene or naphthylene is each independently optionally substituted with one or more R^{3a}, and each R^{3a} are the same or different and each independently represent halogen (e.g., fluorine, chlorine, bromine, or iodine), hydroxy, cyano, amino, nitro, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₁₋₆ alkoxy (e.g., C₁₋₄ alkoxy, such as methoxy, ethoxy, propoxy, isopropoxy, butoxy, sec-butoxy or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—); or
furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, 4H-fluoro[3,2-b]pyrrolylene, pyrrolo[2,1-b]thiazolylene, and imidazo[2,1-b]thiazolylene, with each group being independently optionally substituted with one or more R^{3a}, and each R^{3a} are the same or different and are each independently halogen (e.g., fluorine, chlorine, bromine, or iodine), hydroxy, cyano, amino, nitro, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₁₋₆ alkoxy (e.g., C₁₋₄ alkoxy, such as methoxy, ethoxy, propoxy, isopropoxy, butoxy, sec-butoxy or tert-butoxy), or halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—).

In some embodiments, W¹ is t interconnected rings W² and represented by the following formula:

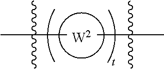

wherein each ring W² are the same or different and each independently represent the following groups:
monocyclic C₃₋₁₀ heterocyclylene group containing one nitrogen atom and optionally a heteroatom selected from nitrogen, oxygen, and sulfur, e.g., piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene or diazacyclooctylene; or
C₅₋₂₀ bridged heterocyclylene group containing one nitrogen atom and optionally a heteroatom selected from nitrogen, oxygen, and sulfur, e.g., azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene or diazabicyclo[2.2.2]octanylene, such as the following groups:

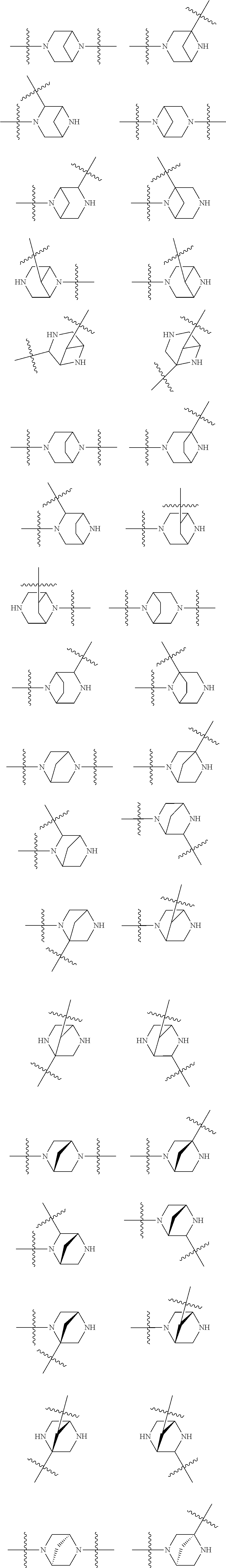

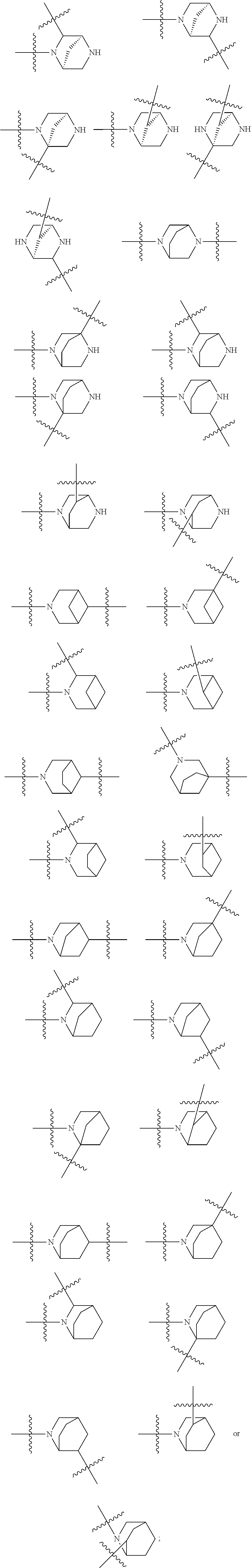

C₃₋₁₅ cycloalkylene, e.g., cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, C₅₋₁₅ spiro-cycloalkylene, adamantanylene, noradamantanylene, bornylene, bicyclo[2.2.1]heptylene or bicyclo[2.2.1]heptenylene;
wherein the monocyclic C₃₋₁₀ heterocyclylene, C₅₋₂₀ bridged heterocyclylene, and C₃₋₁₅ cycloalkylene are each independently optionally substituted with one or more substituents selected from the group consisting of deuterium, hydroxy, amino, mercapto, nitro, halogen (e.g., fluorine, chlorine, bromine, or iodine), cyano, optionally deuterated C₁₋₆ alkyl, halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally deuterated cyclopropyl, cyclobutyl, cyclopentyl, or cyclohexyl), optionally deuterated C₁₋₆ alkoxy (e.g., C₁₋₄ alkoxy, such as methoxy, ethoxy, propoxy, isopropoxy, butoxy, sec-butoxy or tert-butoxy), optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof; and
t represents an integer of 1 or 2.

In some embodiments, W¹ represents the following groups:

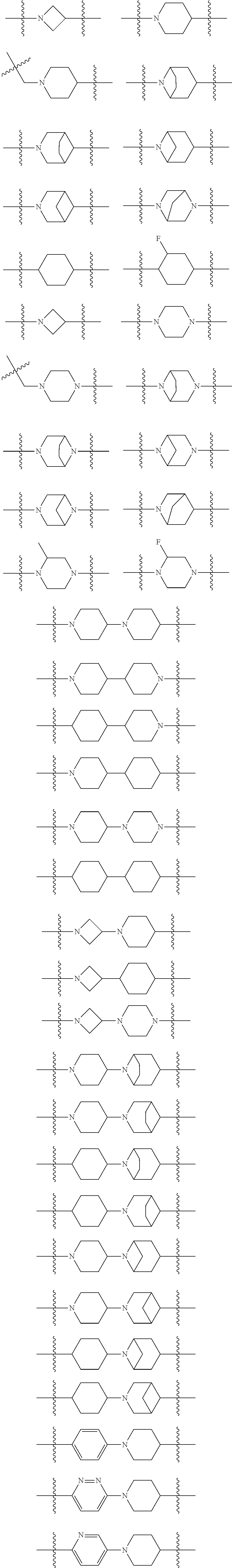

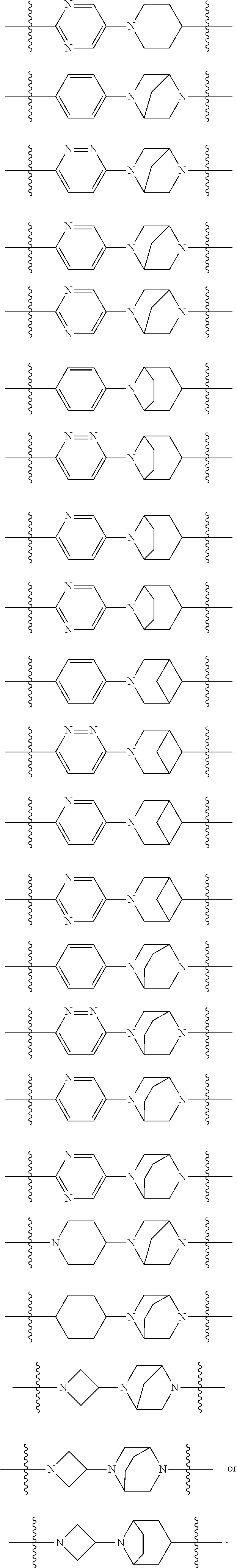

wherein the groups are optionally substituted with one or more substituents selected from the group consisting of deuterium, halogen (e.g., fluorine, chlorine, bromine, or iodine), cyano, amino, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—), deuterated C₁₋₆ alkoxy, or any combination thereof.

In some embodiments, R¹—W¹ represents the following groups:

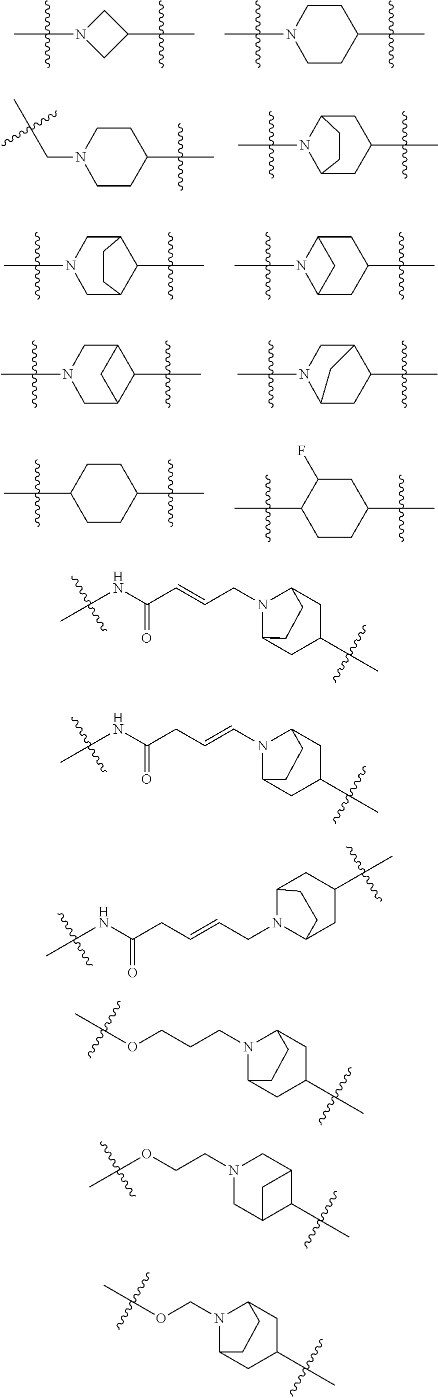

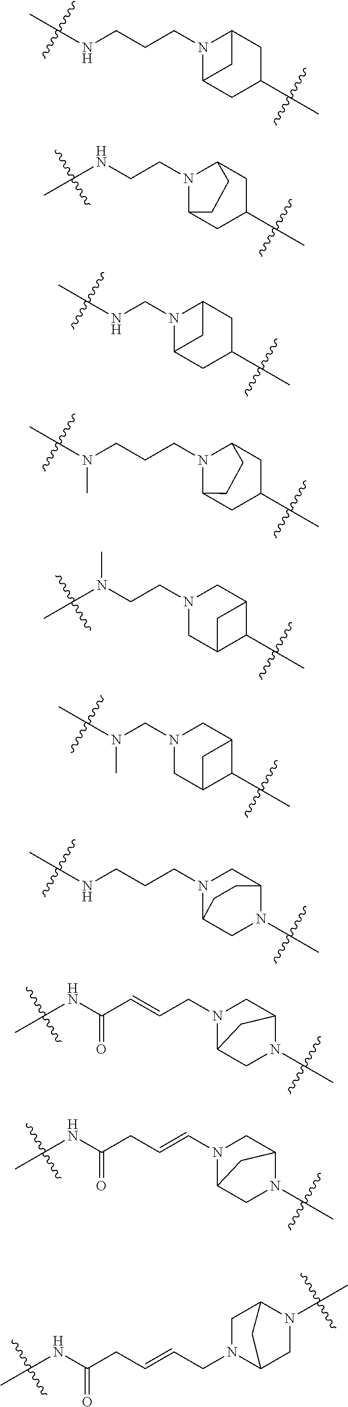

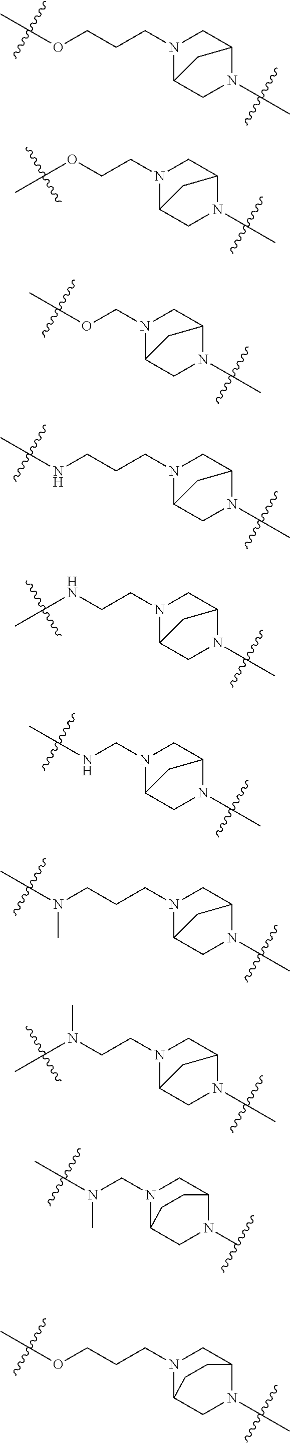

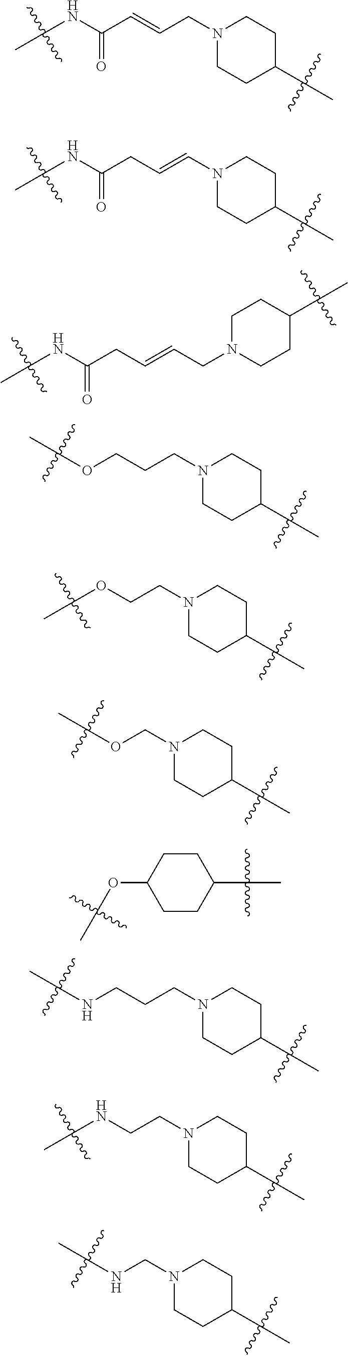

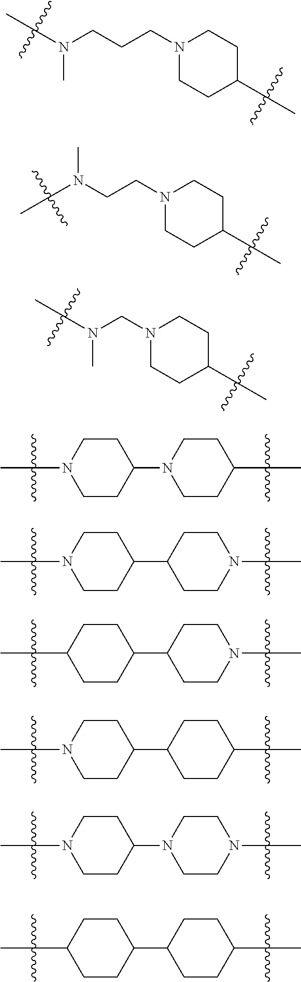

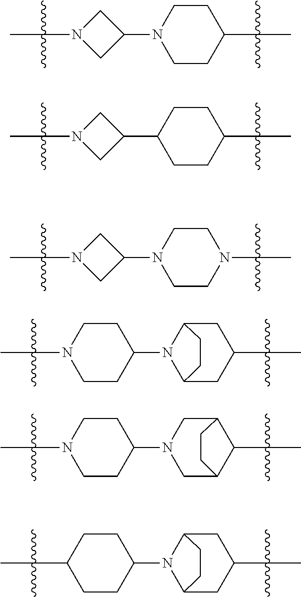

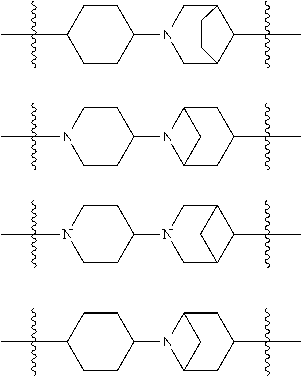

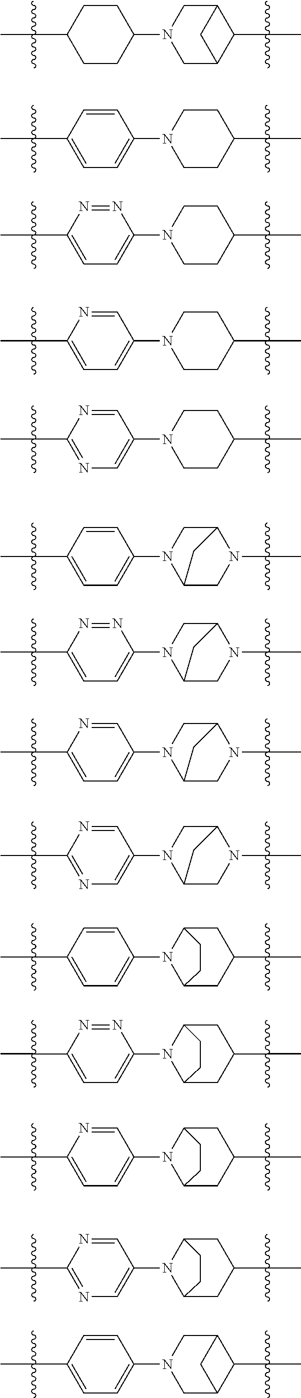

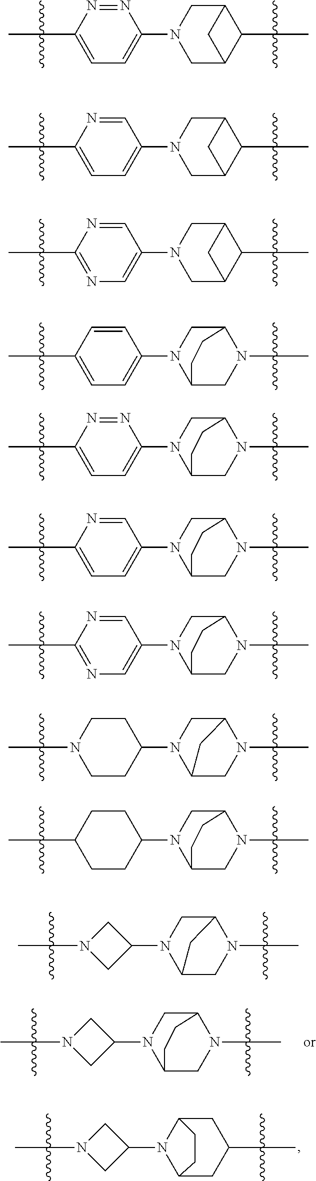

wherein the groups are optionally substituted with one or more substituents selected from the group consisting of deuterium, halogen (e.g., fluorine, chlorine, bromine, or iodine), cyano, amino, C₁₋₆ alkyl (e.g., C₁₋₆ alkyl, C₁₋₅ alkyl or C₁₋₄ alkyl, such as methyl, ethyl, propyl, isopropyl, butyl, sec-butyl, tert-butyl, pentyl or hexyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₆ alkyl, perdeuterated C₁₋₅ alkyl or perdeuterated C₁₋₄ alkyl, such as CD₃, CD₃CD₂-, CD₃CD₂CD₂- etc.), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—), deuterated C₁₋₆ alkoxy, or any combination thereof.

In some embodiments, ULM represents the structures of Formula (ULM-1), Formula (ULM-2), Formula (ULM-3), Formula (ULM-4), Formula (ULM-5), Formula (ULM-6), Formula (ULM-7), Formula (ULM-8), Formula (ULM-9), or Formula (ULM-10):

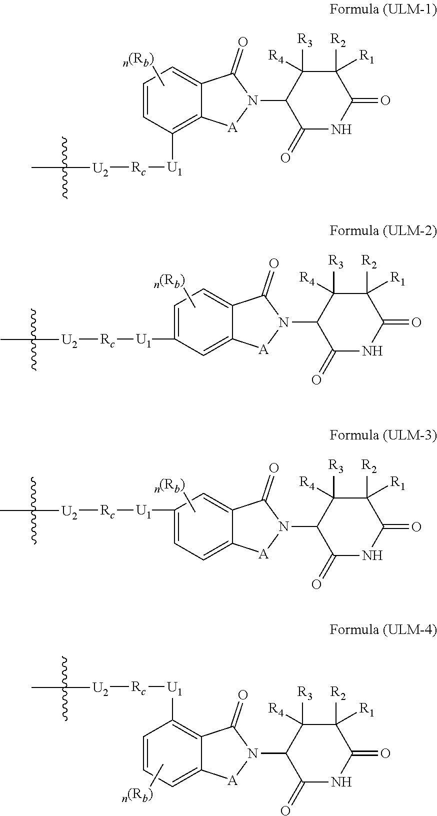

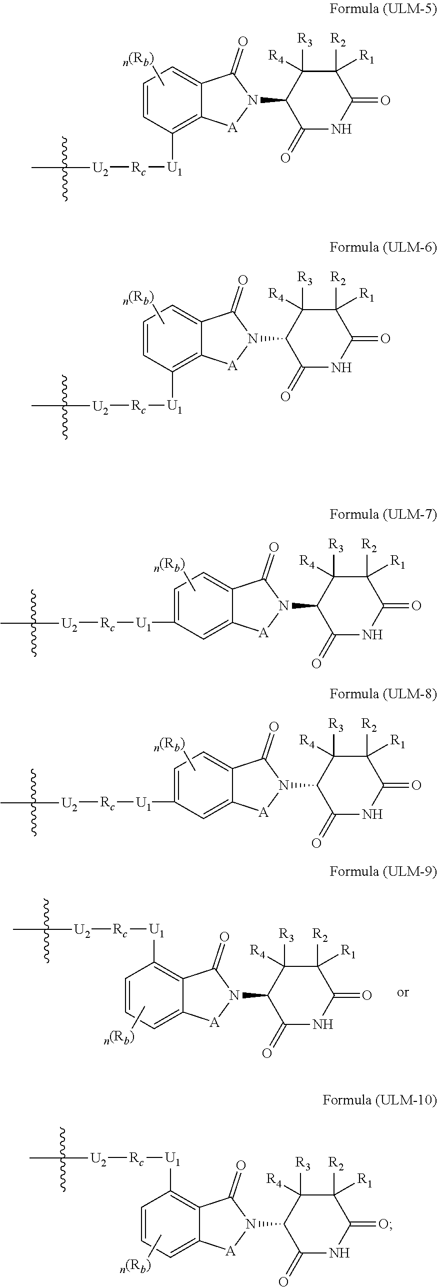

wherein A represents CO, CH₂ or CD₂;
R₁, R₂, R₃ and R₄ are the same or different and each independently represent hydrogen, deuterium, halogen (e.g., fluorine, chlorine, bromine, or iodine), optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), or halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—);
(R_{b})ₙ indicates that benzene ring is optionally substituted with n R_{b} groups, and each R_{b} are the same or different and each independently represent hydrogen, deuterium, halogen (e.g., fluorine, chlorine, bromine, or iodine), hydroxy, mercapto, nitro, amino, cyano, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), C₂₋₆ alkenyl or C₂₋₆ alkynyl;
n represents an integer of 0, 1, 2 or 3;
U₁ and U₂ are the same or different and each independently represent a bond, N(R_{d}) or O, wherein R_{d} represents H or optionally deuterated C₁₋₆ alkyl (e.g., optionally deuterated C₁₋₄ alkyl, such as methyl, CD₃, ethyl, propyl, isopropyl, butyl, sec-butyl or tert-butyl);
R_{c} represents monocyclic C₃₋₁₀ heterocyclylene group containing one nitrogen atom and optionally a heteroatom selected from nitrogen, oxygen, and sulfur, e.g., piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene or diazacyclooctylene; or
R_{c} represents C₅₋₂₀ bridged heterocyclylene group containing one nitrogen atom and optionally a heteroatom selected from nitrogen, oxygen, and sulfur, e.g., azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene or diazabicyclo[2.2.2]octanylene, such as the following groups:

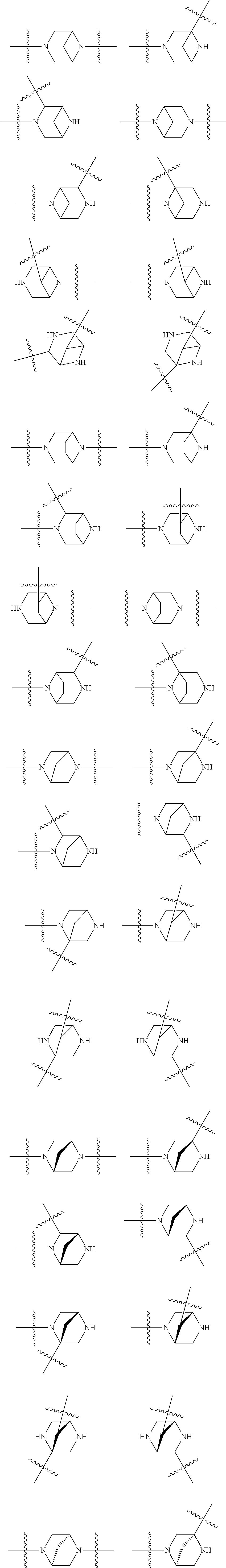

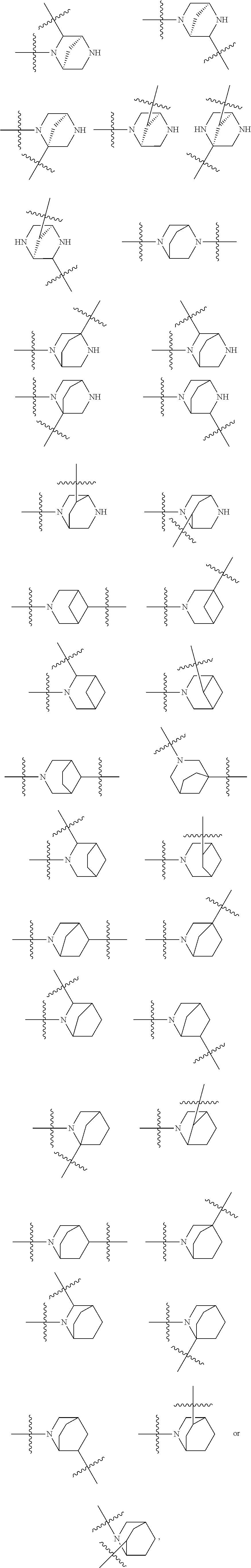

wherein the monocyclic C₃₋₁₀ heterocyclylene and C₅₋₂₀ bridged heterocyclylene are each independently optionally substituted with one or more substituents selected from the group consisting of deuterium, hydroxy, amino, mercapto, nitro, halogen (e.g., fluorine, chlorine, bromine, or iodine), cyano, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally deuterated cyclopropyl, cyclobutyl, cyclopentyl, or cyclohexyl), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof.

In some embodiments, R_{c} represents the following groups:

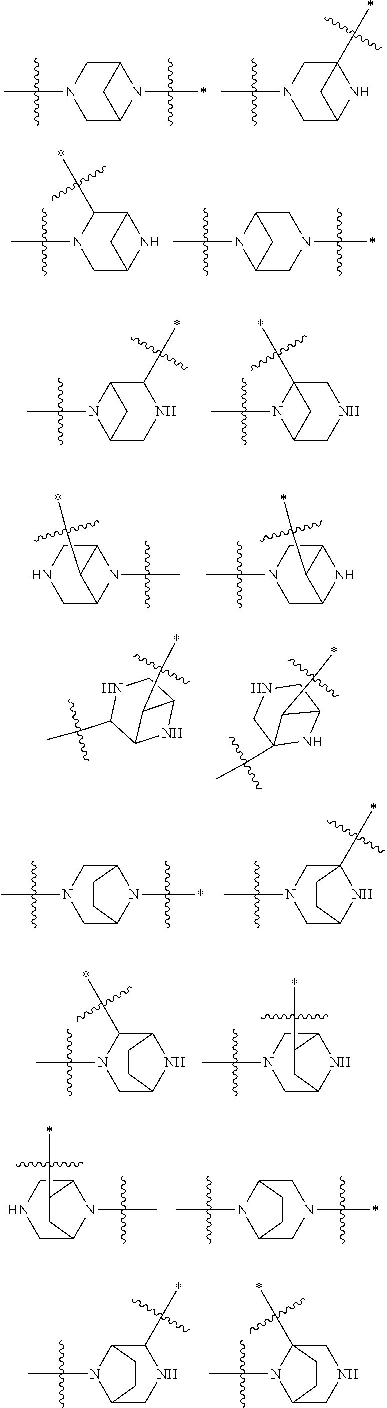

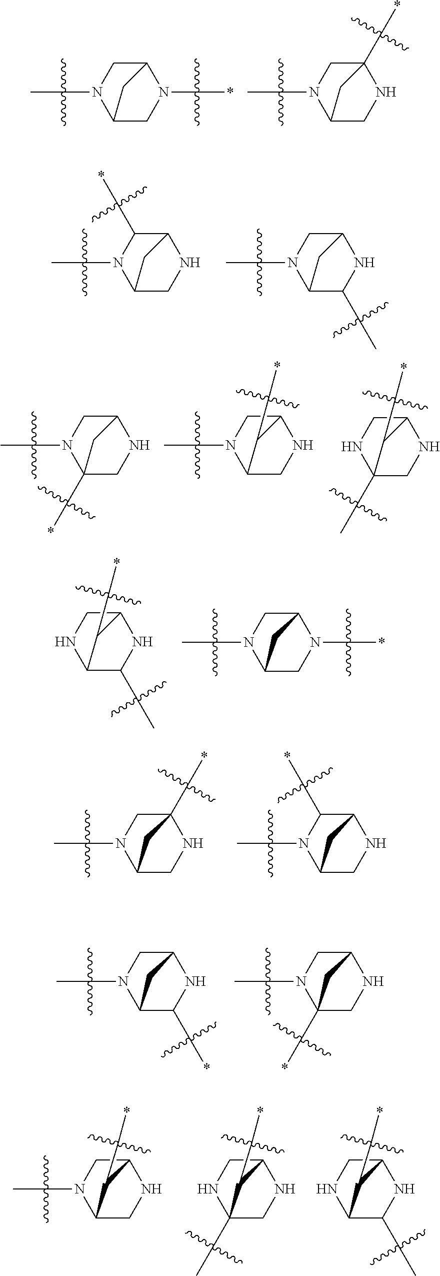

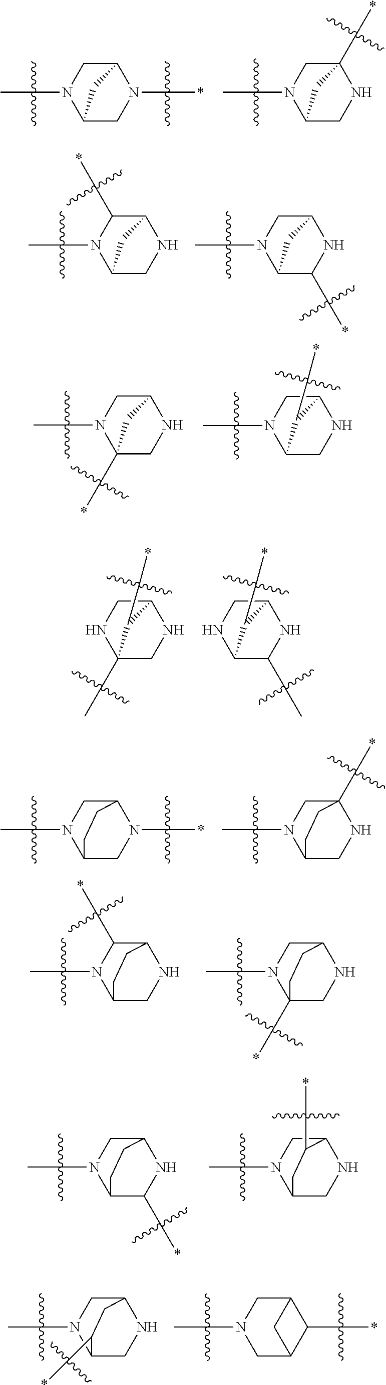

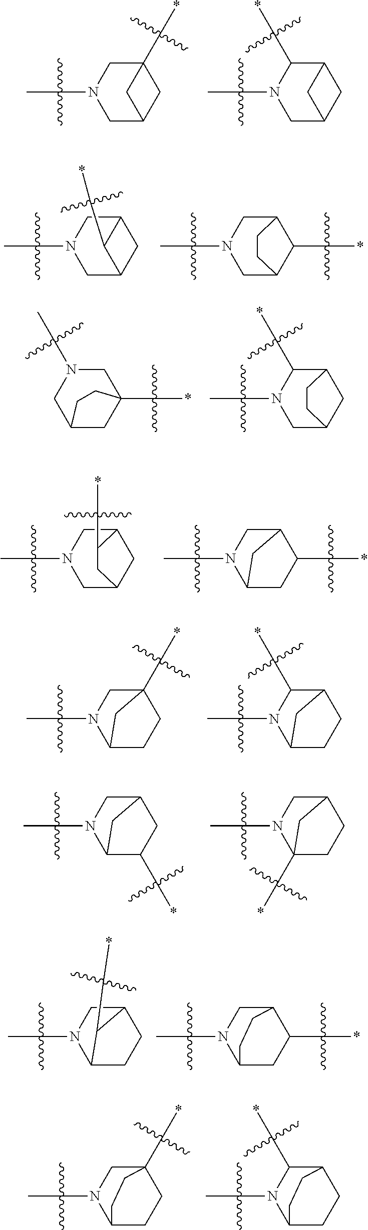

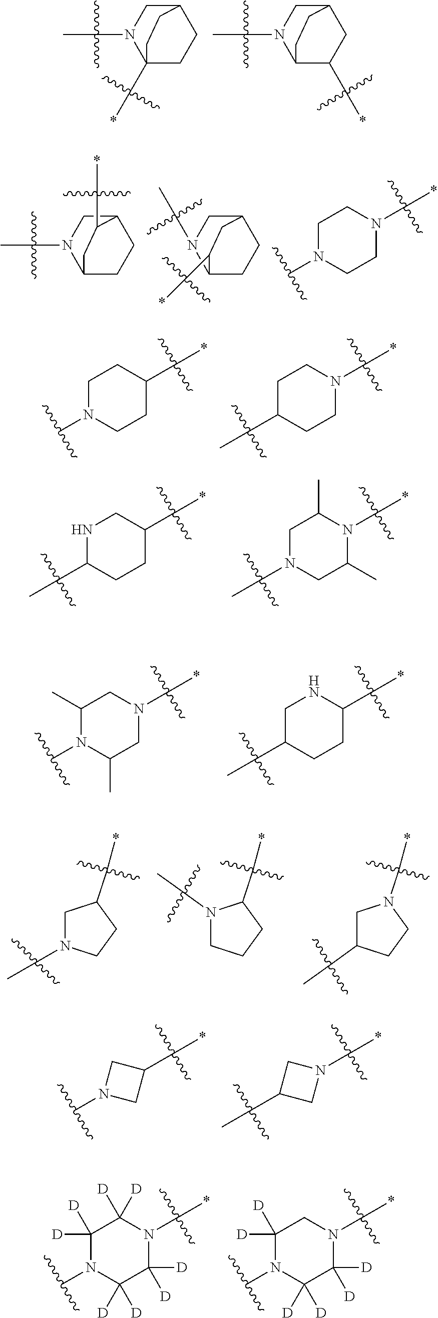

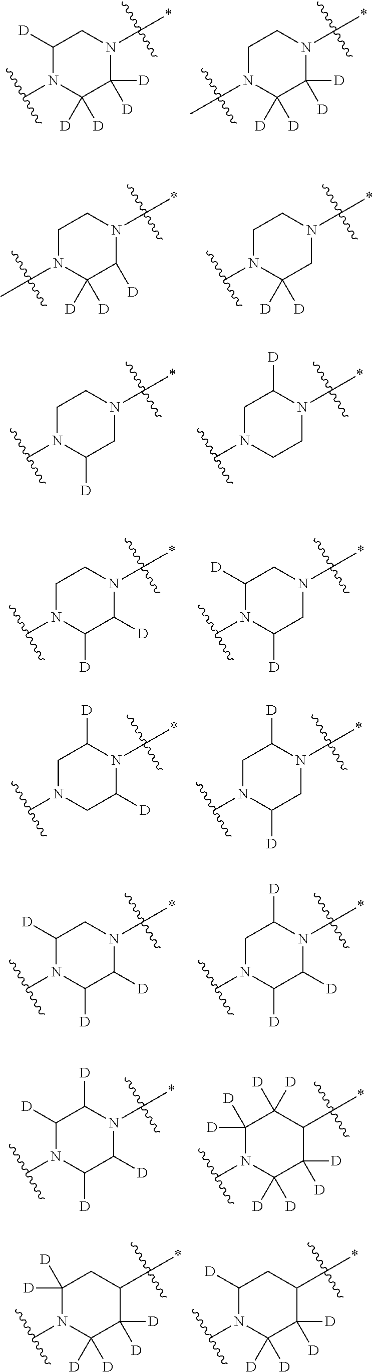

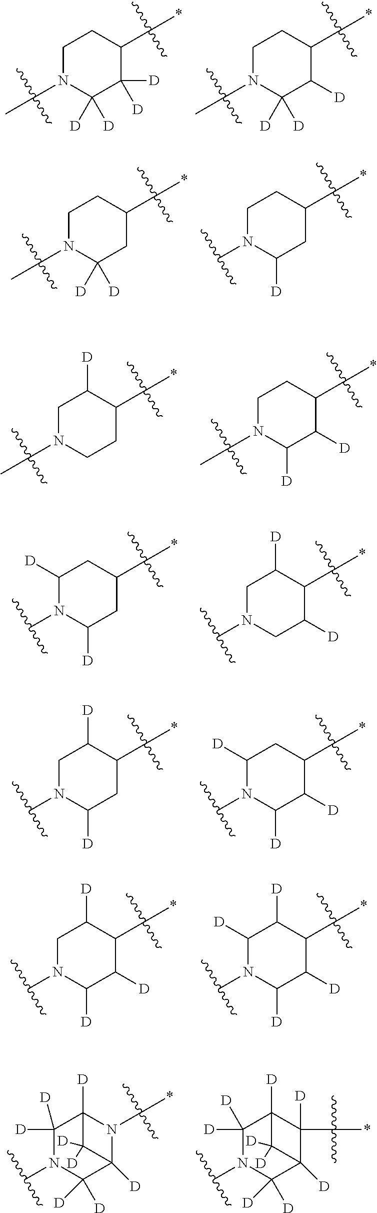

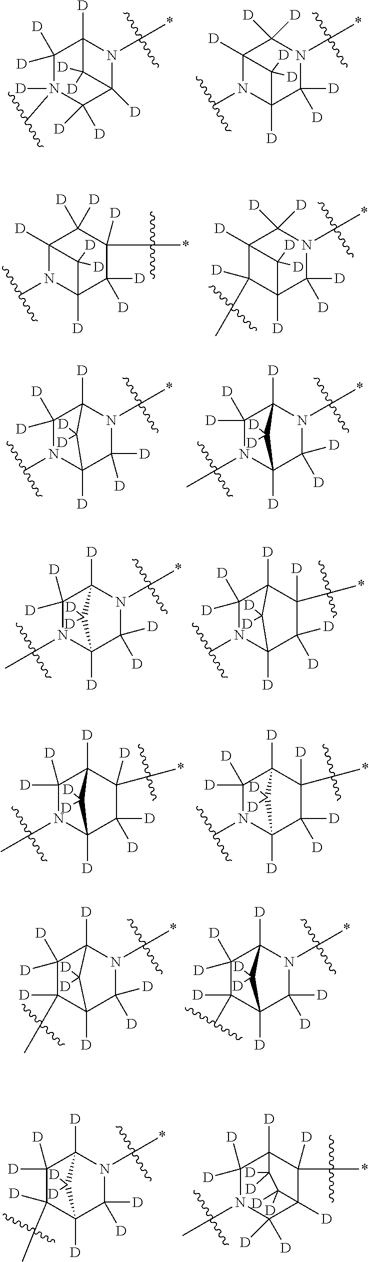

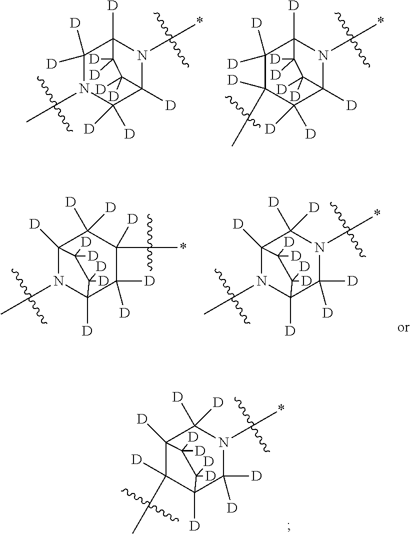

wherein symbol * indicates the point of attachment to U₁.

In some embodiments, ULM represents the structures of Formula (ULM-11), Formula (ULM-12), Formula (ULM-13), Formula (ULM-14), Formula (ULM-15), Formula (ULM-16), Formula (ULM-17), Formula (ULM-18), Formula (ULM-19), Formula (ULM-20), Formula (ULM-21) or Formula (ULM-22):

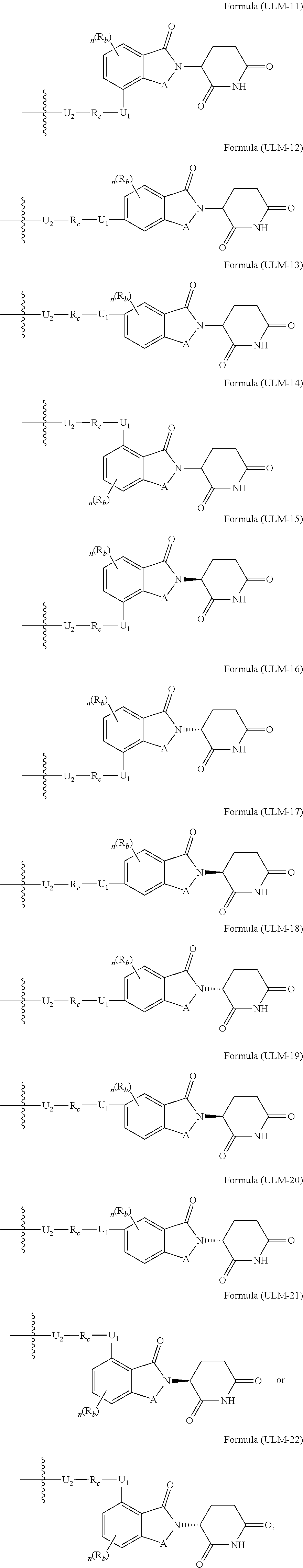

wherein A, (R_{b})ₙ, R_{b}, R_{c}, U₁, U₂ are as defined above.

In some embodiments, ULM represents the structure of the following formula:

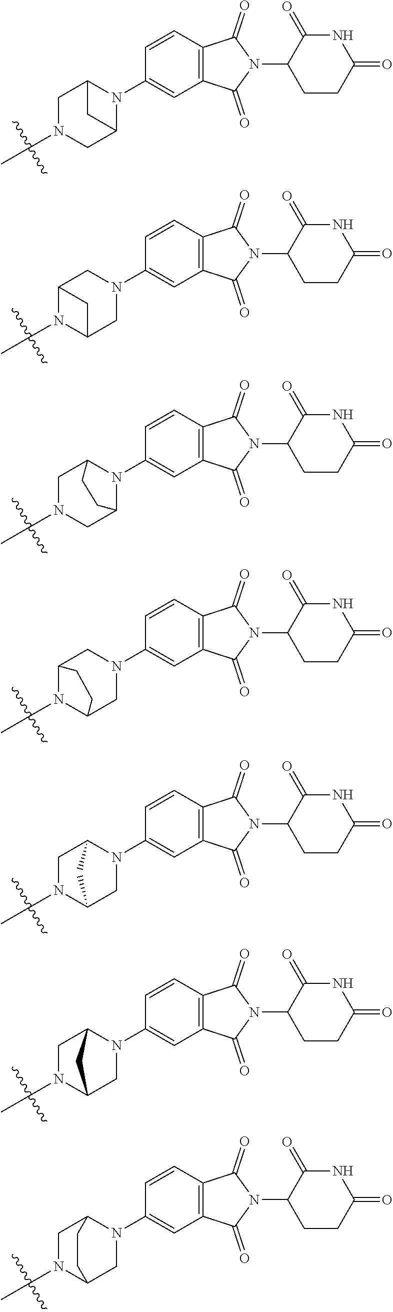

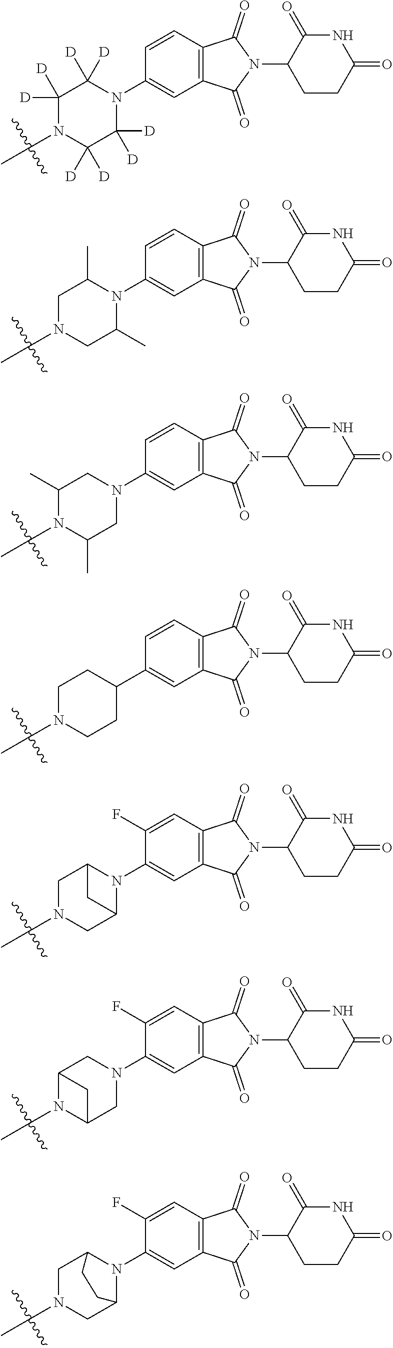

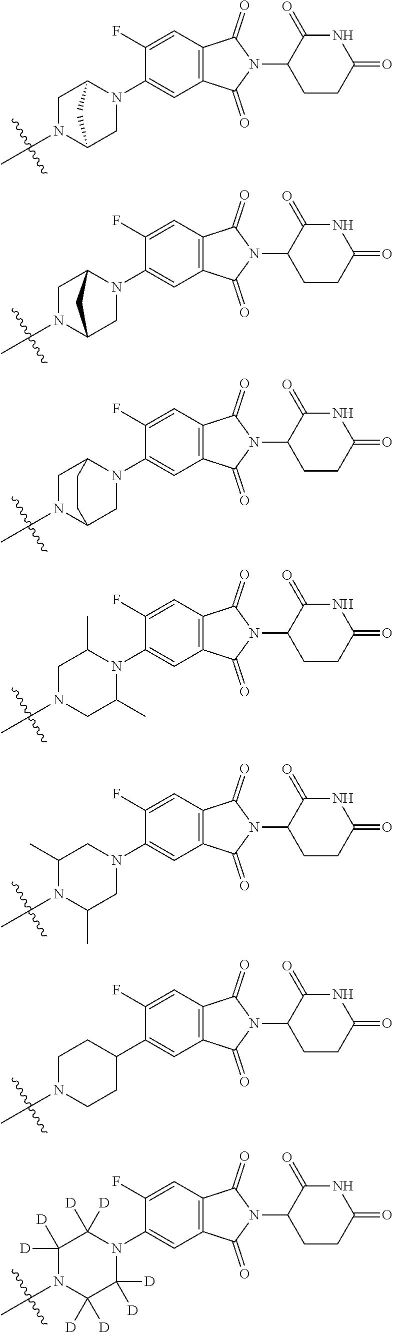

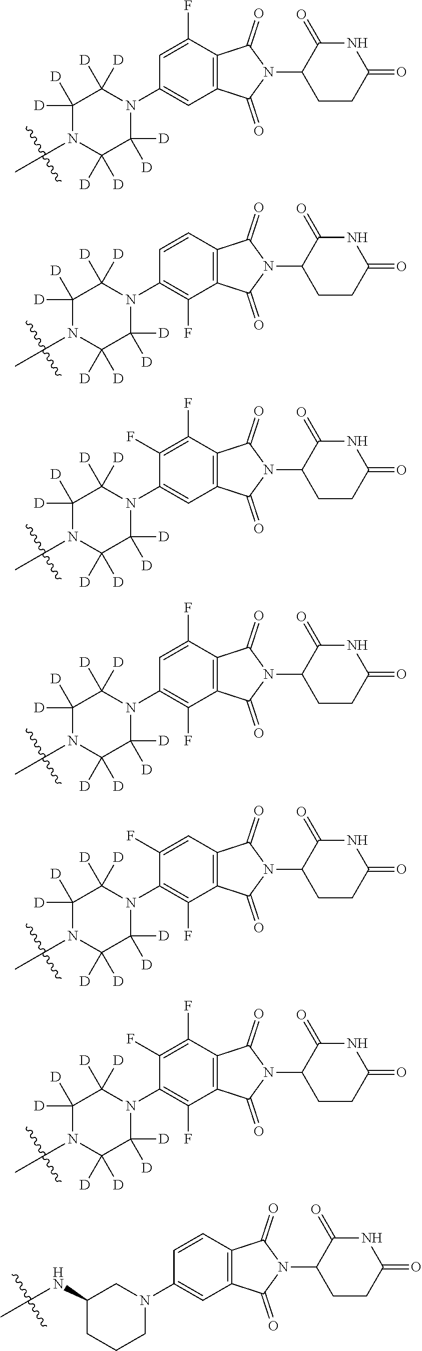

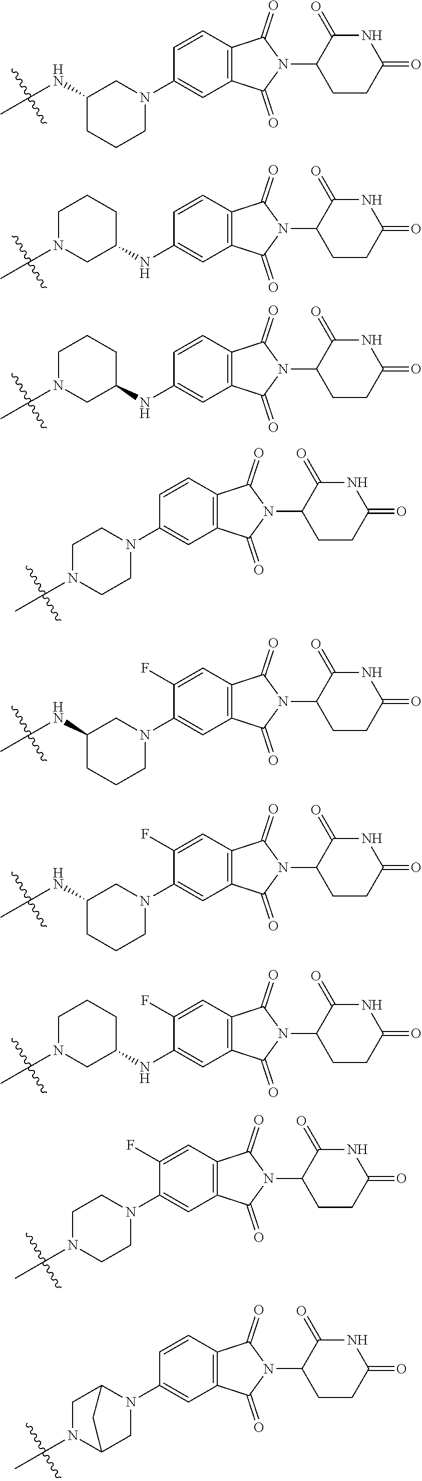

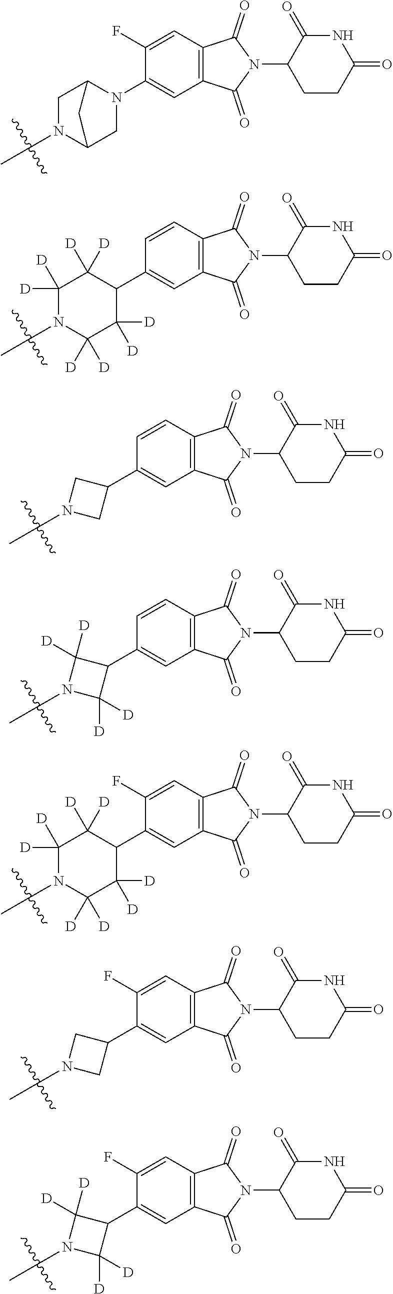

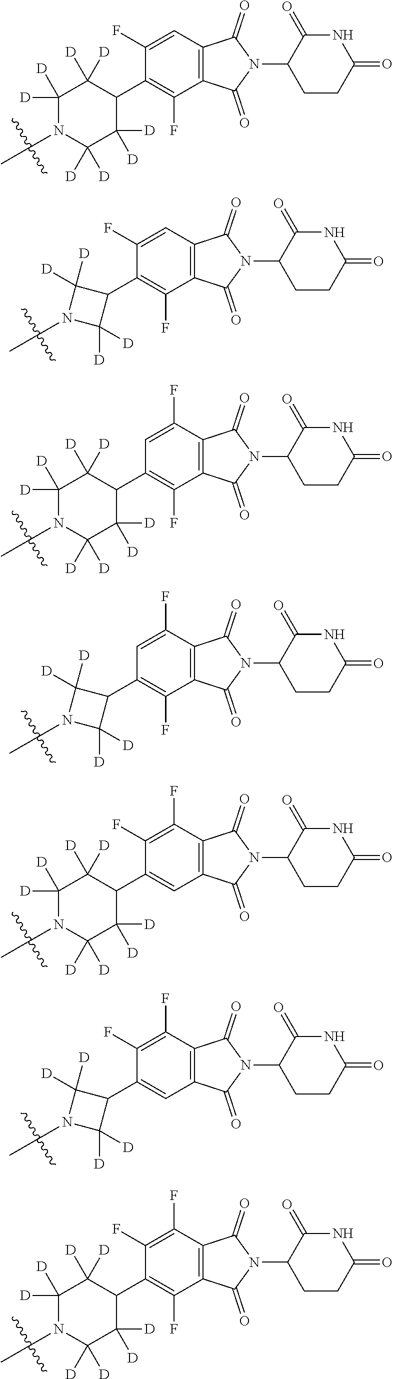

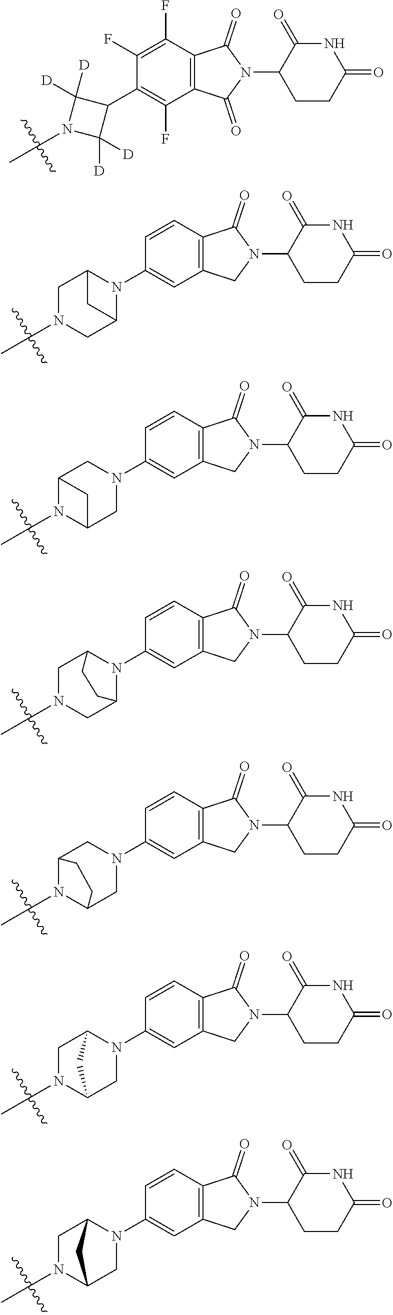

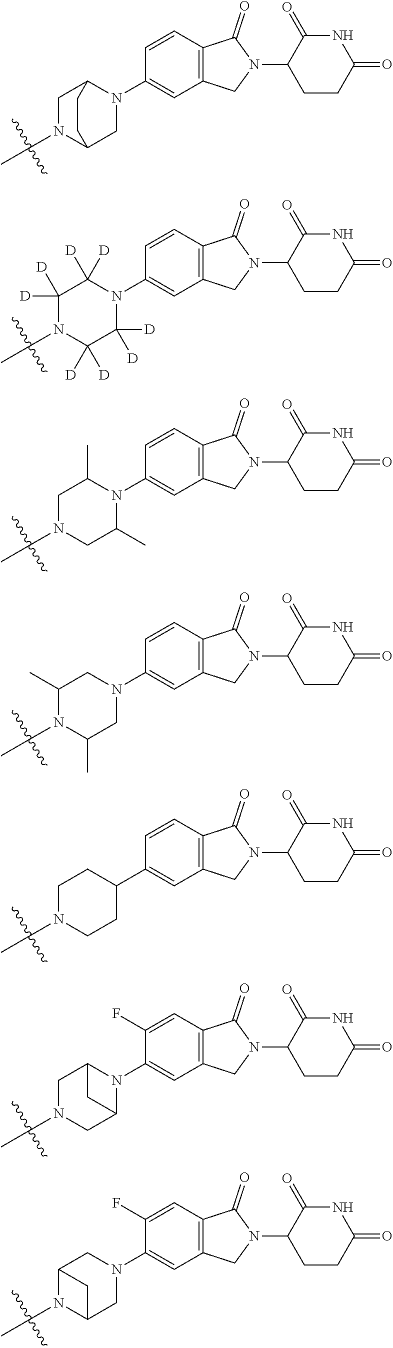

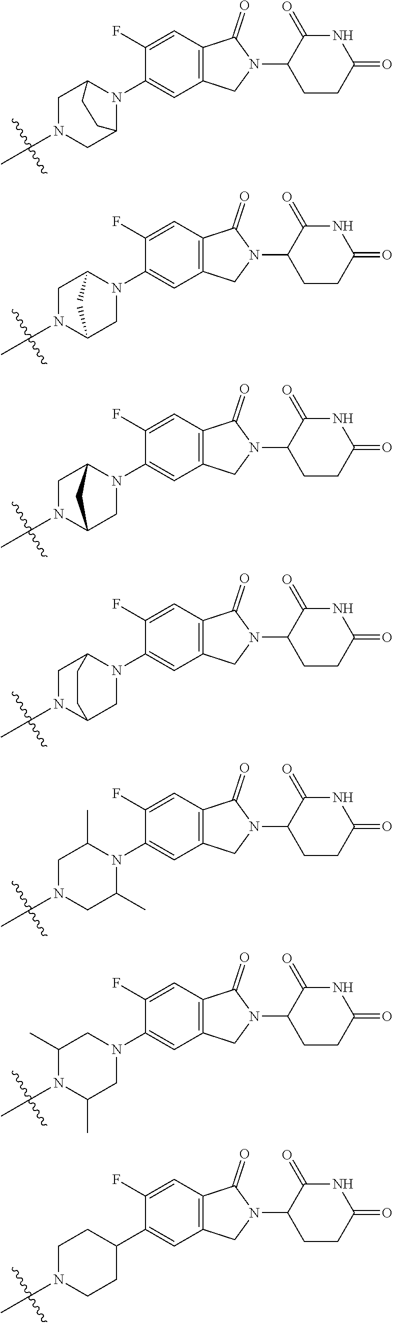

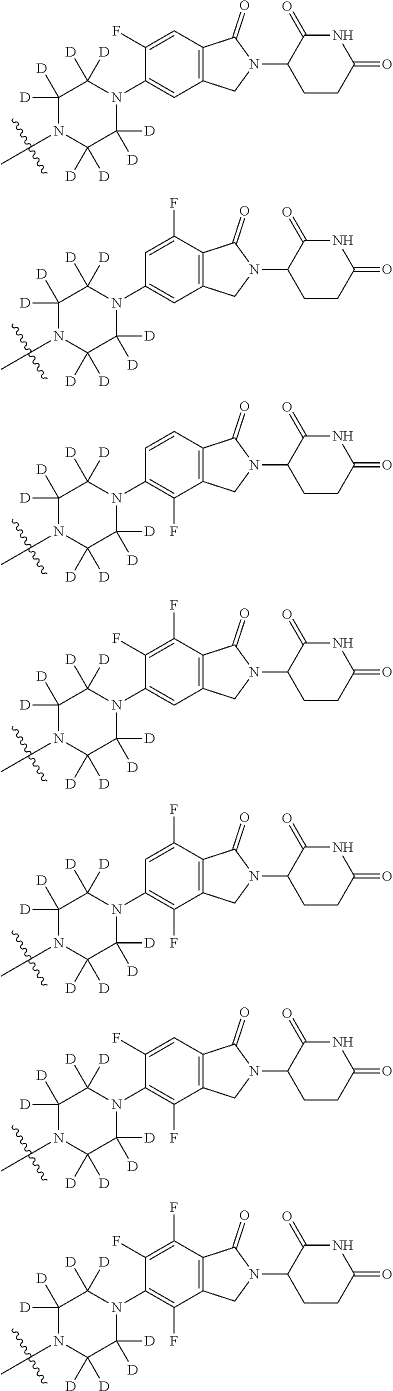

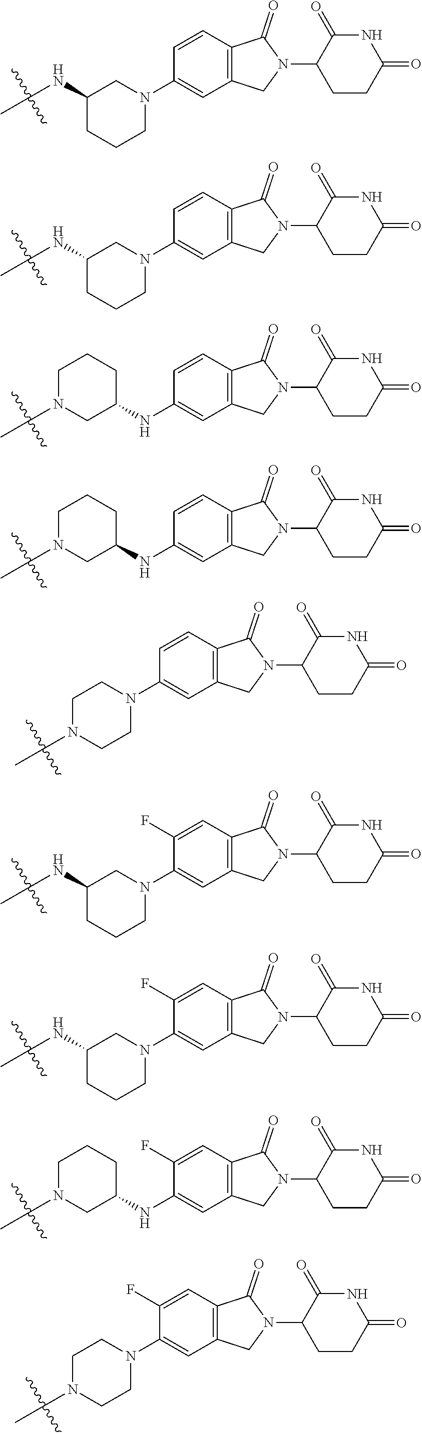

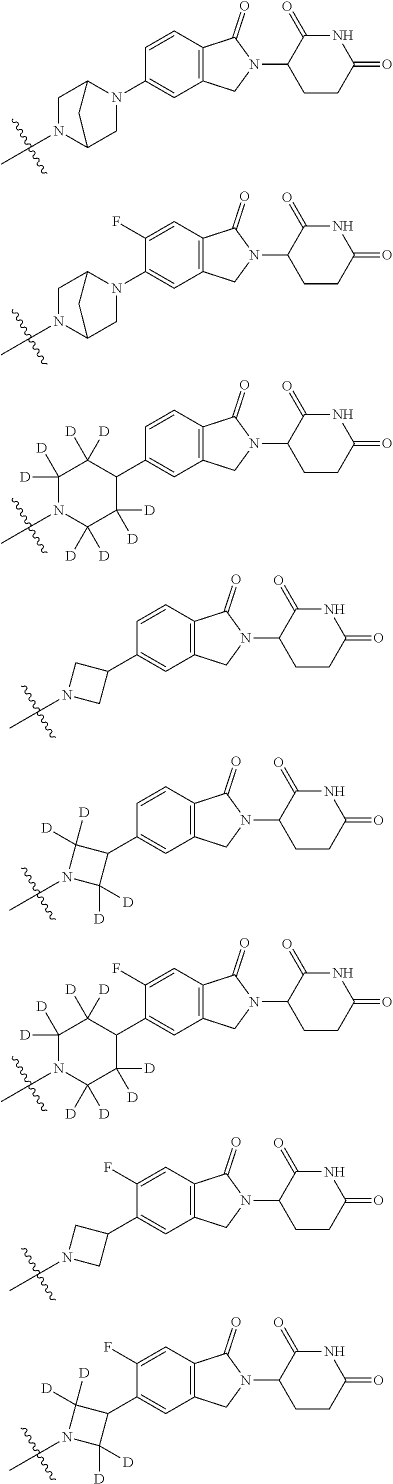

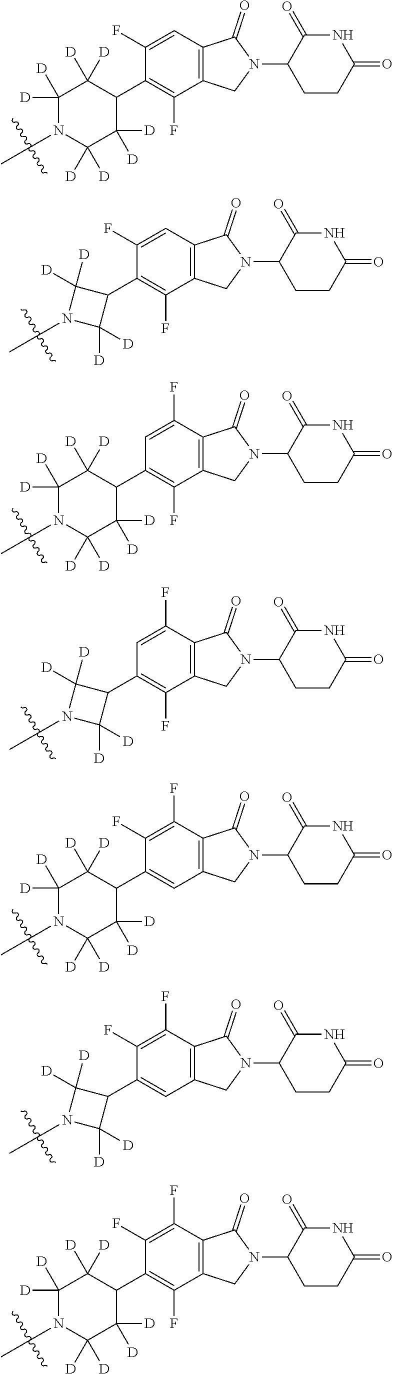

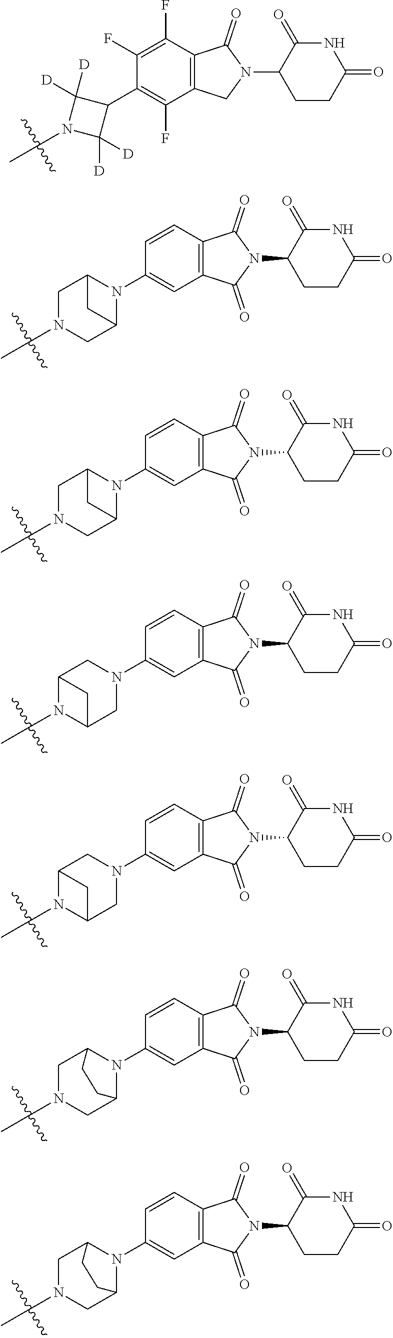

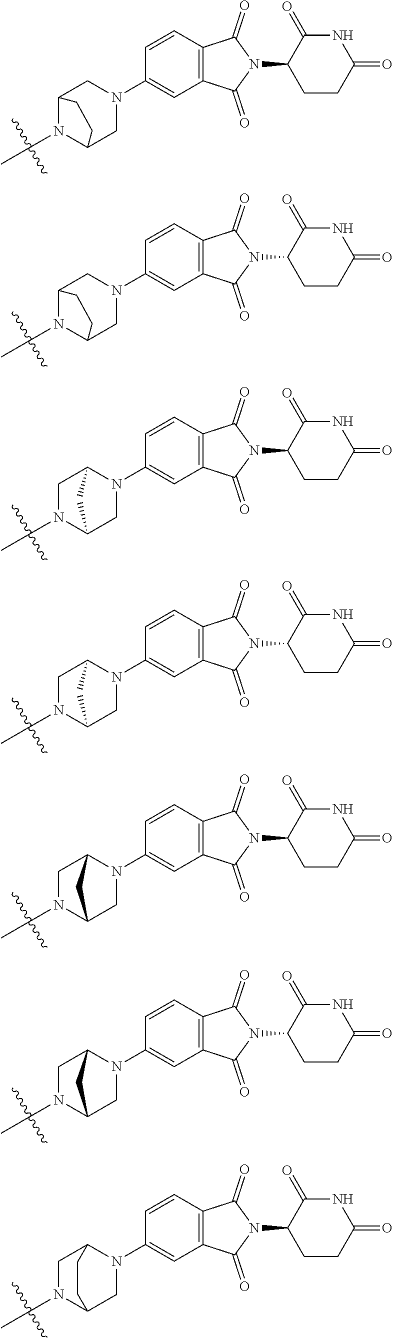

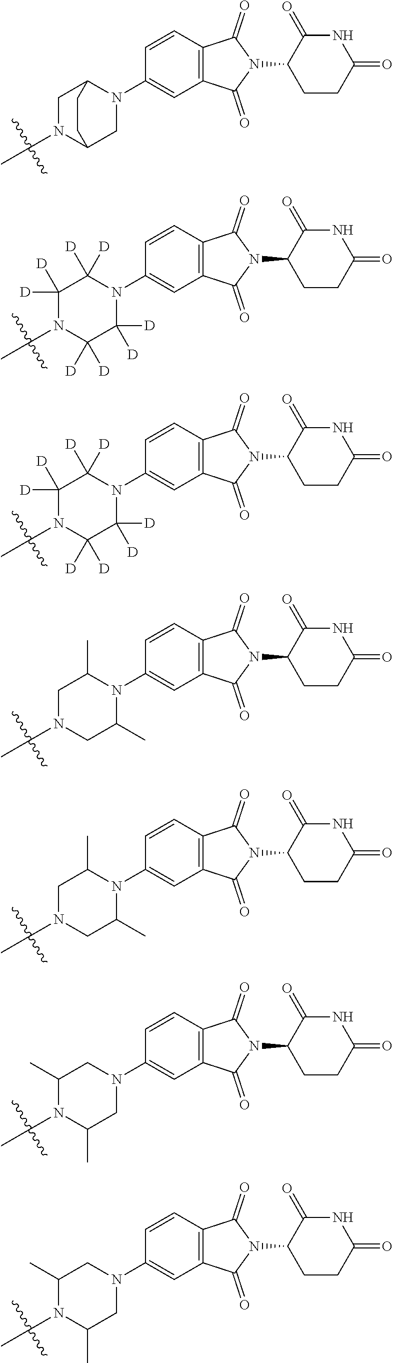

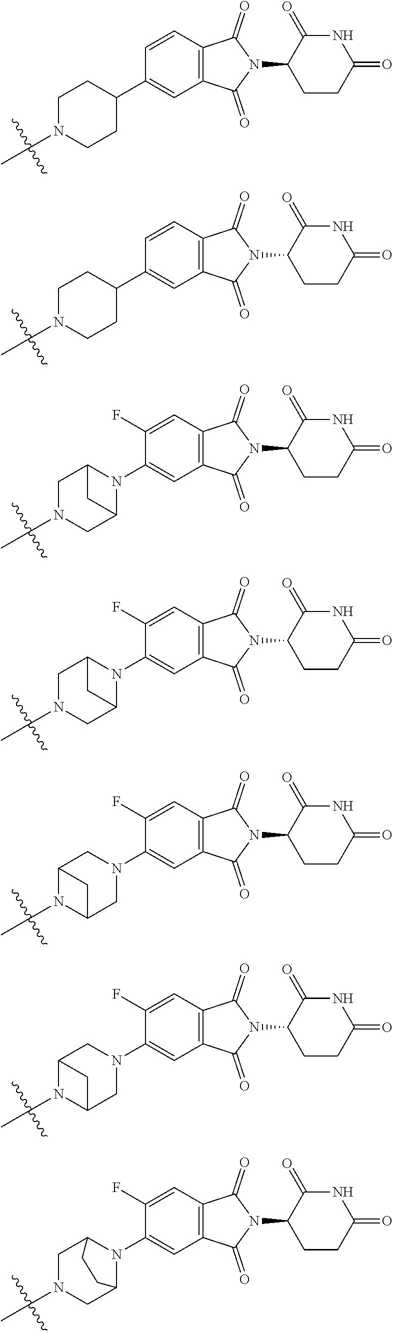

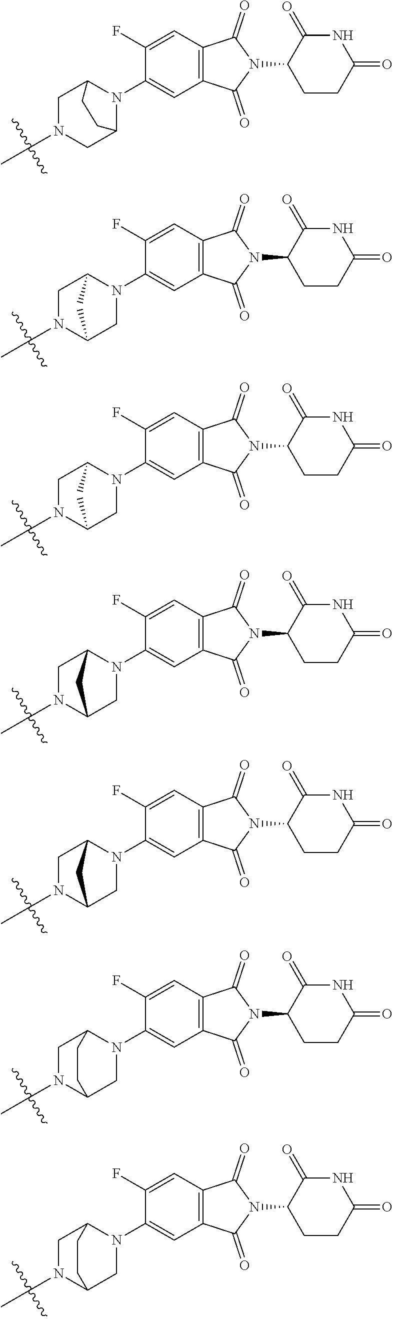

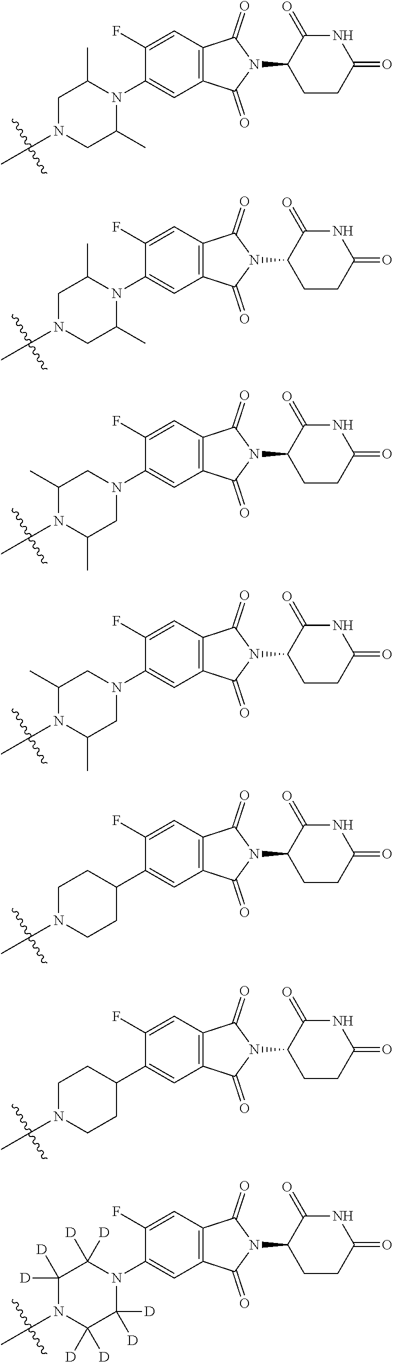

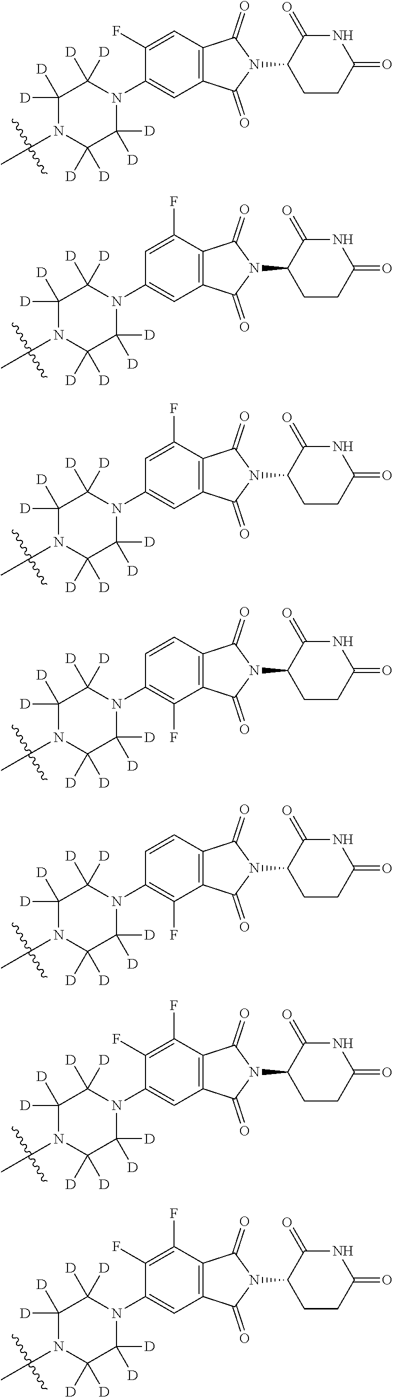

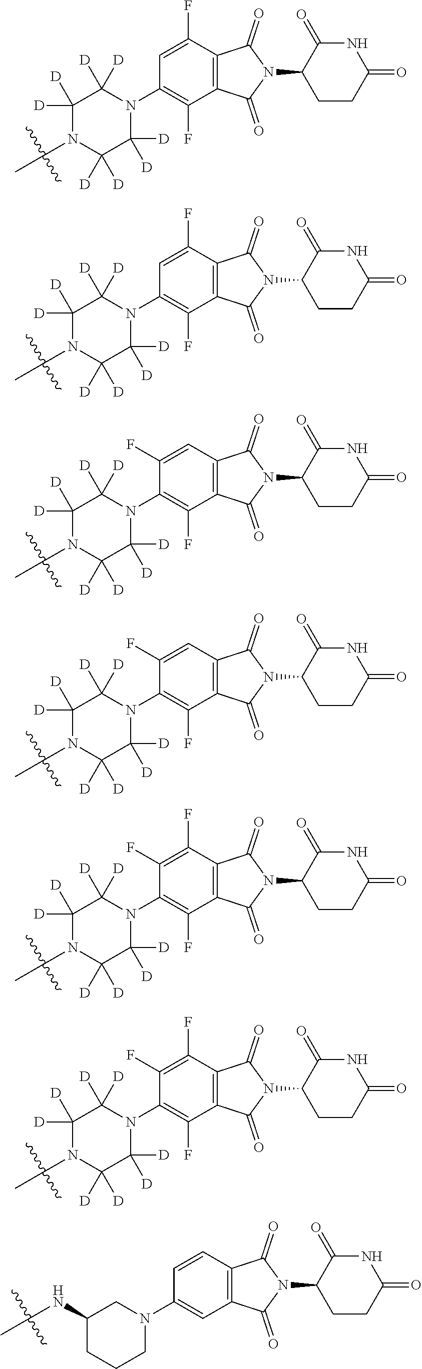

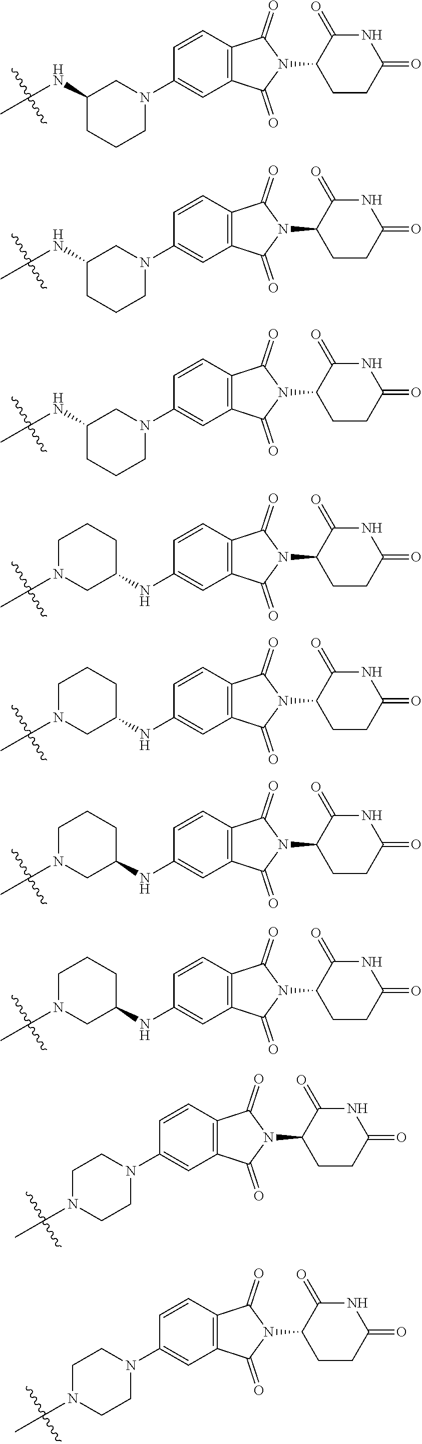

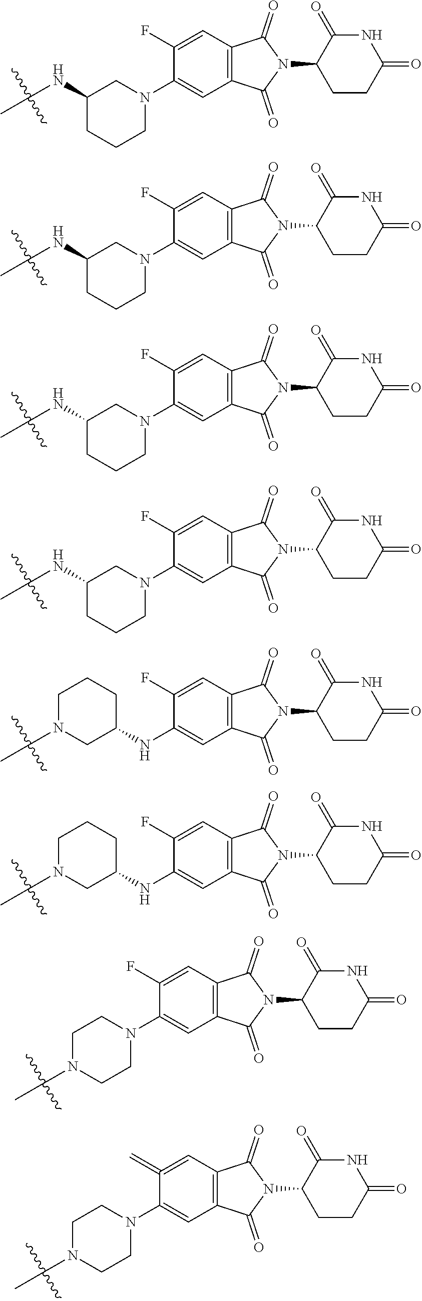

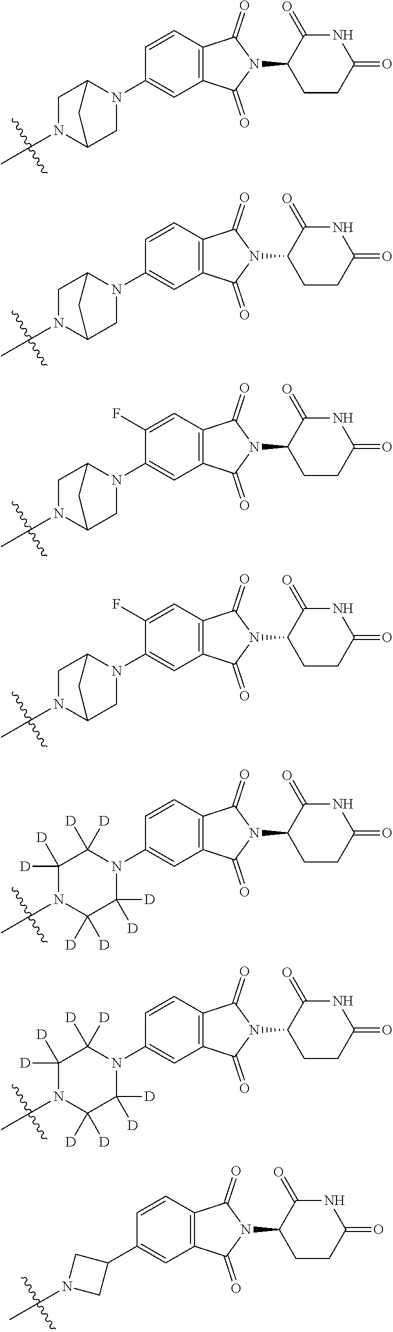

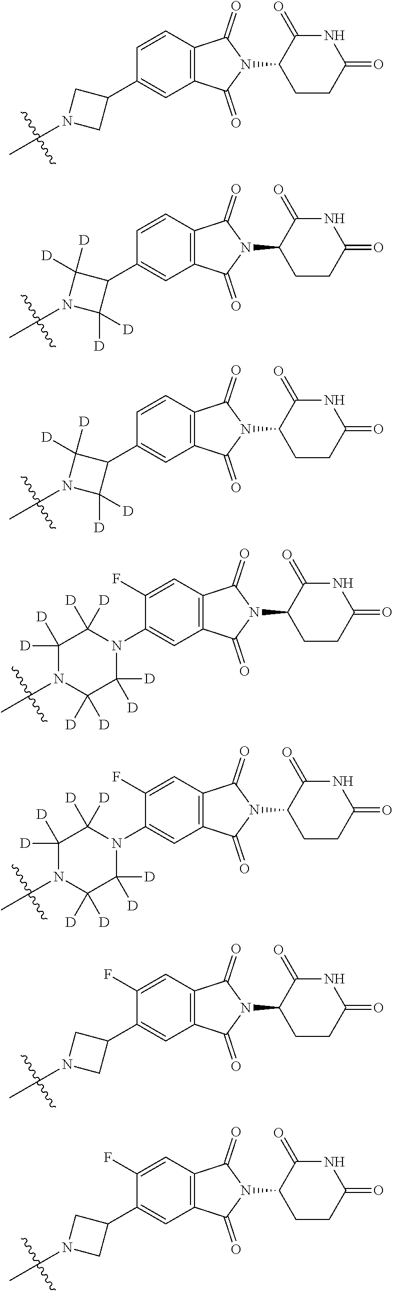

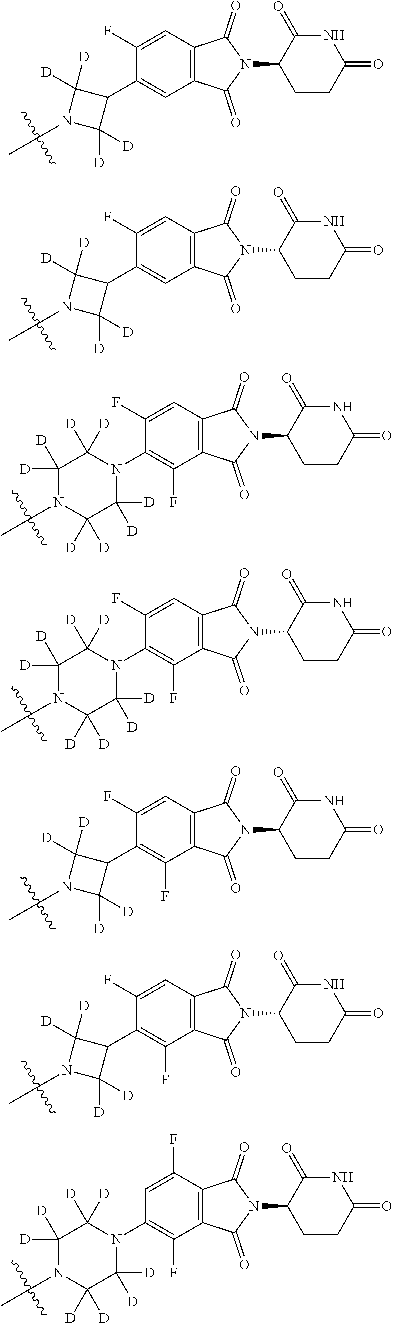

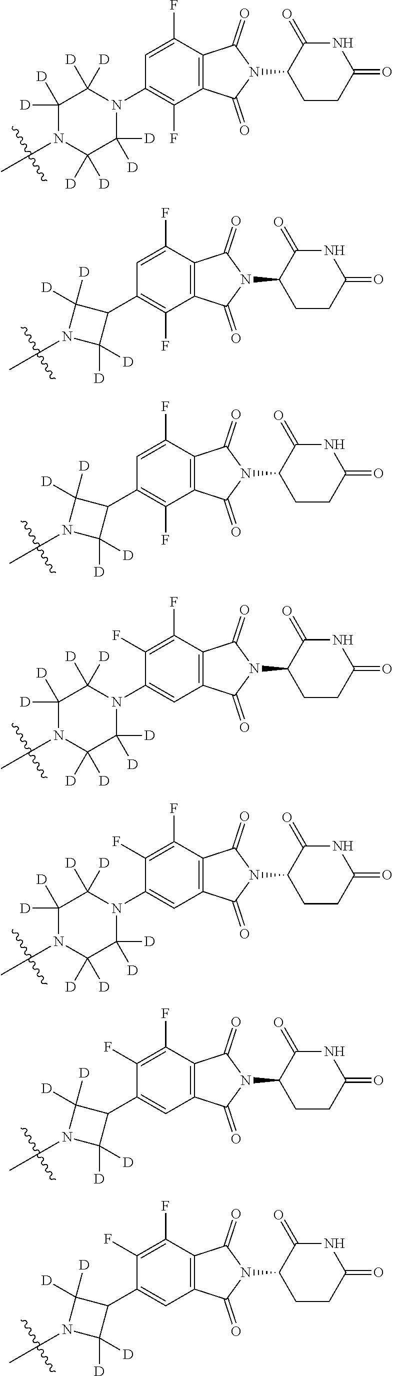

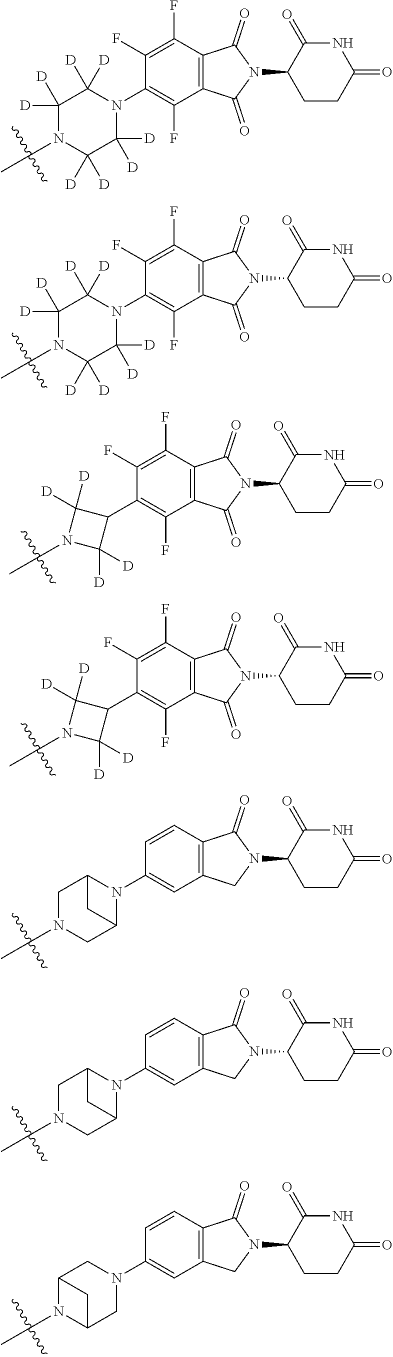

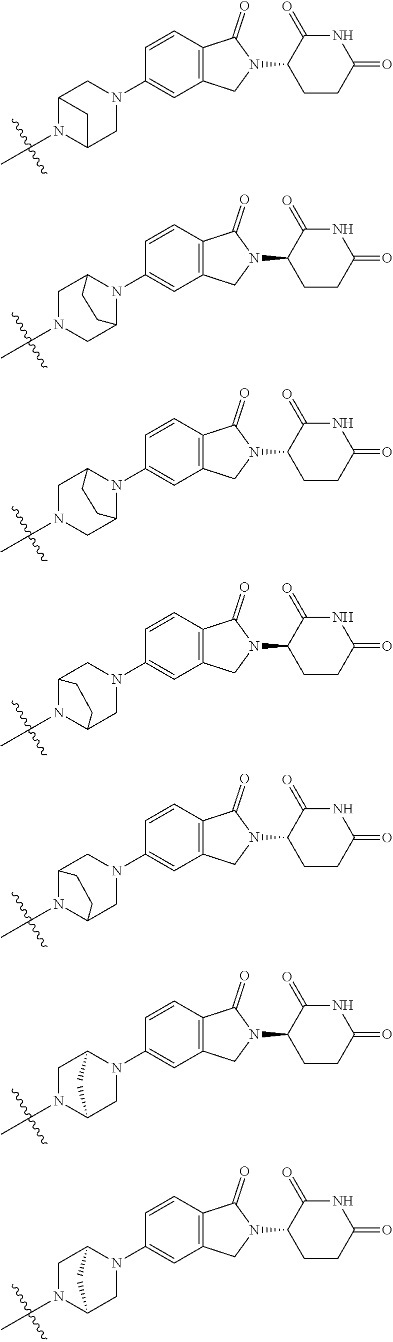

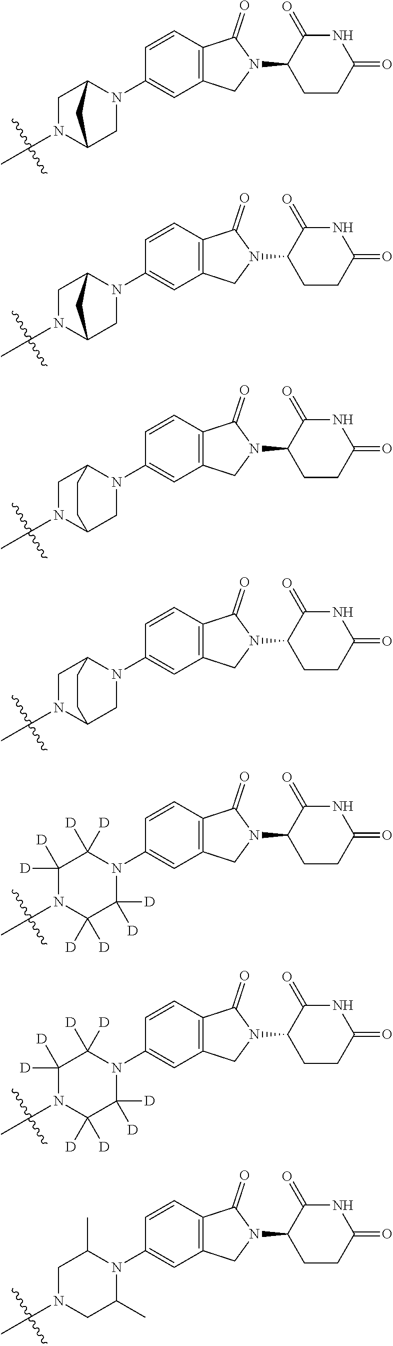

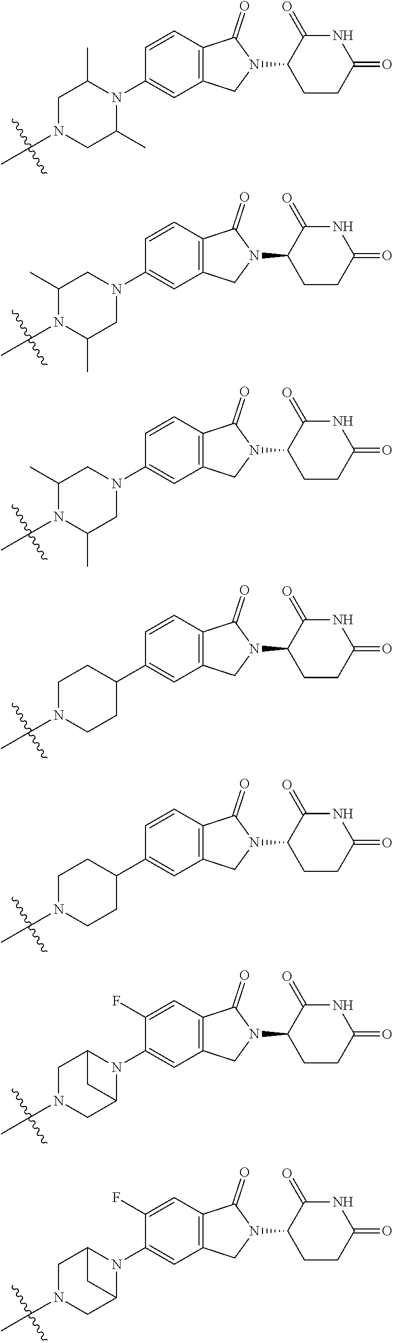

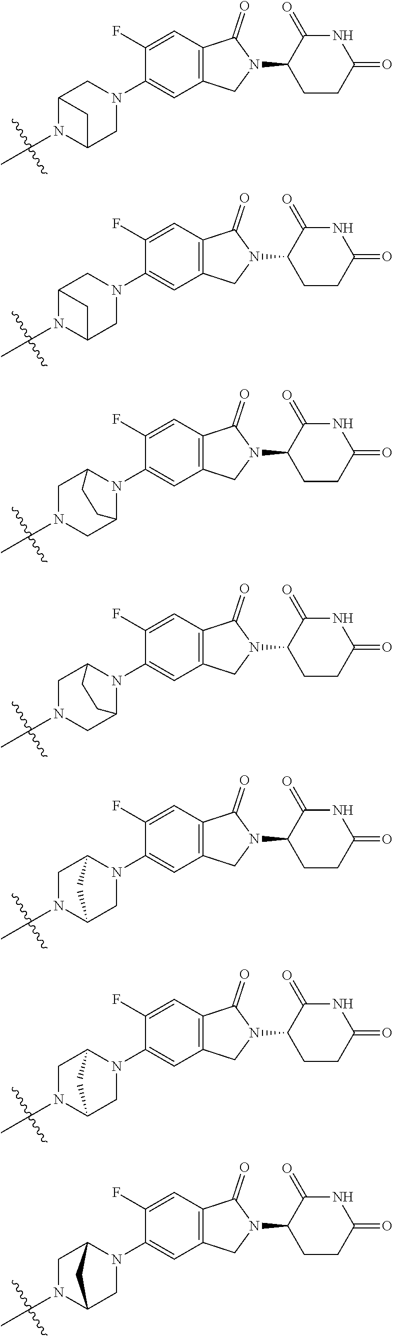

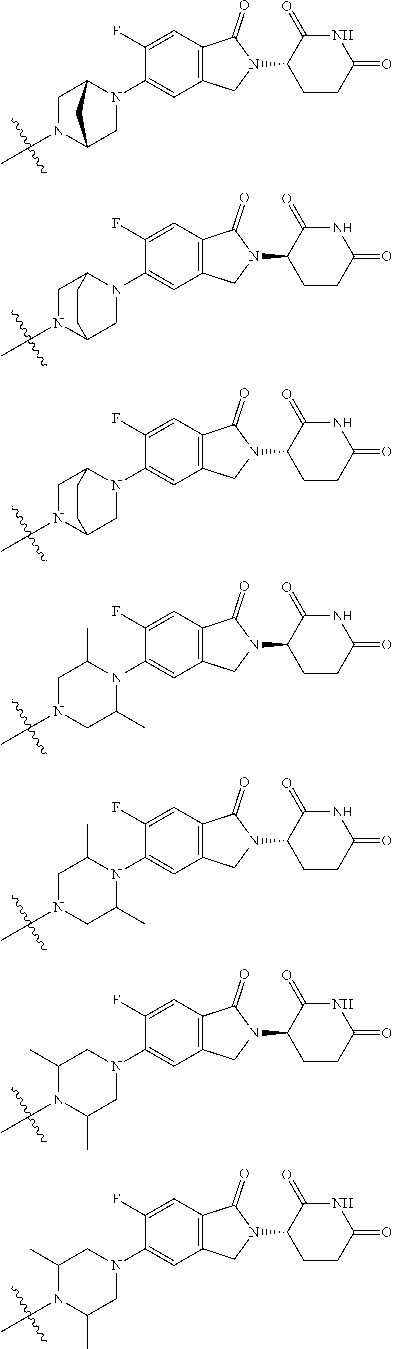

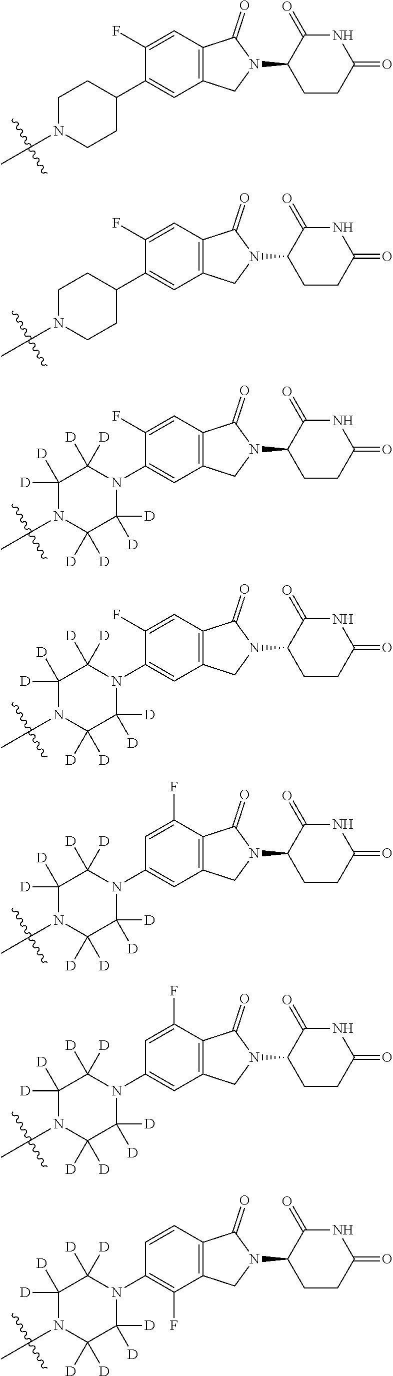

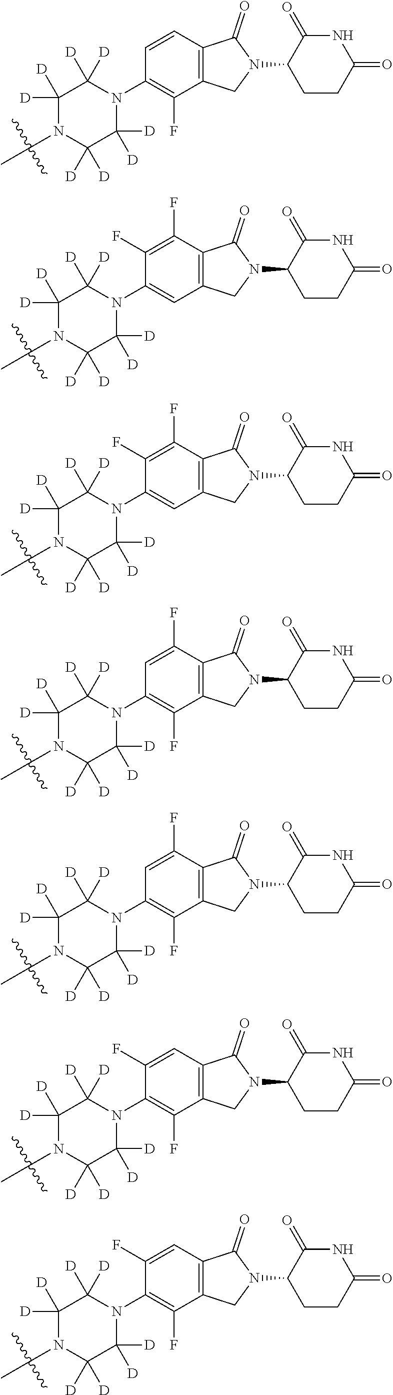

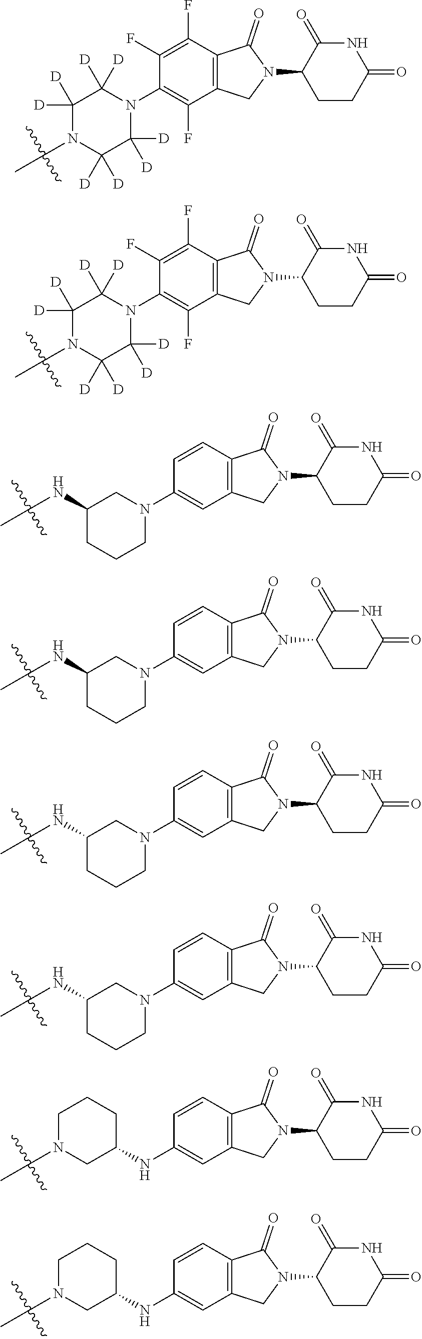

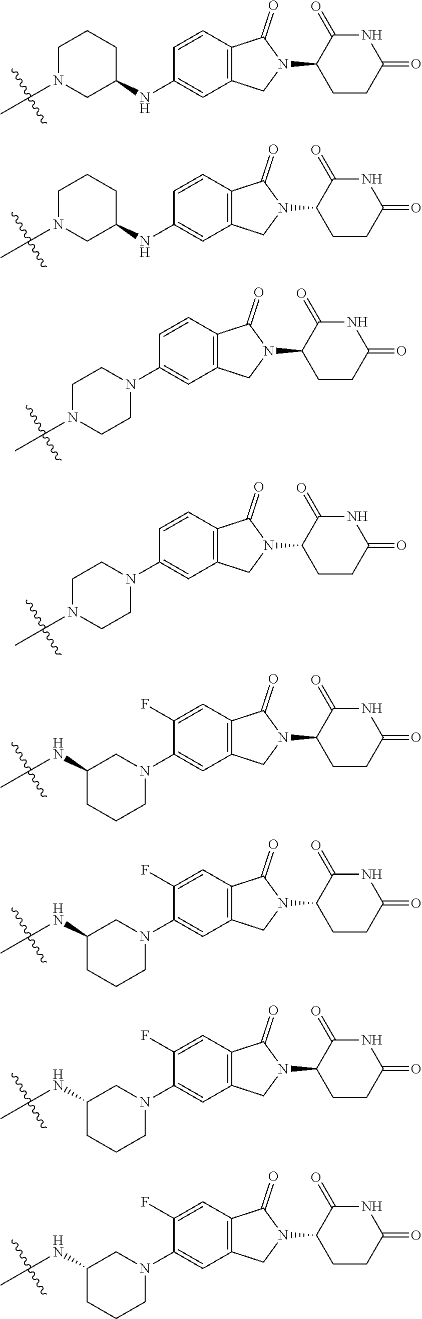

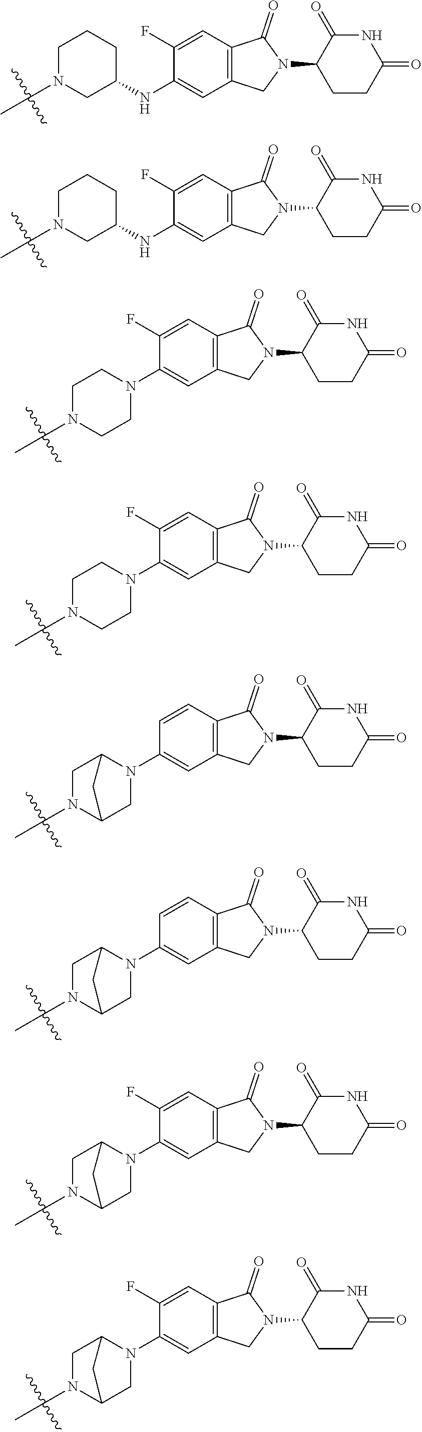

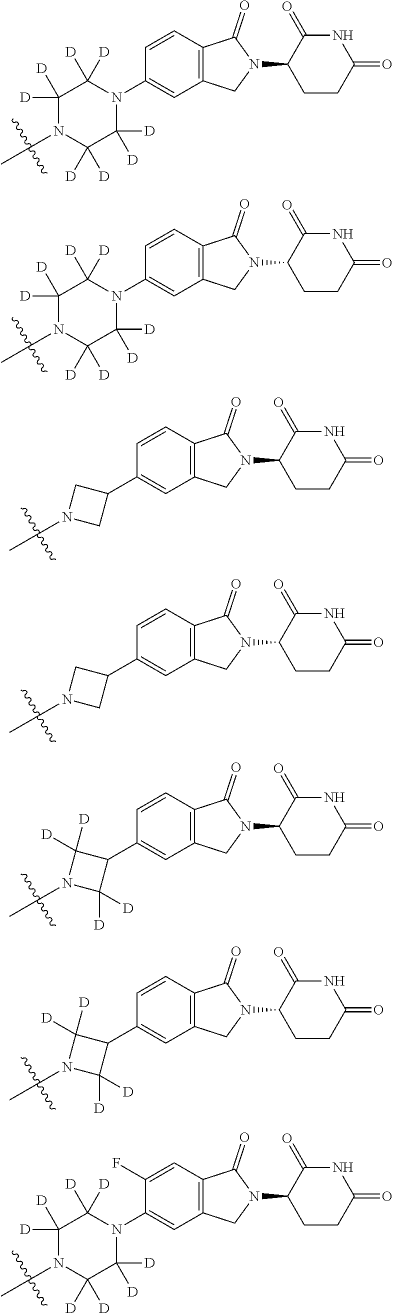

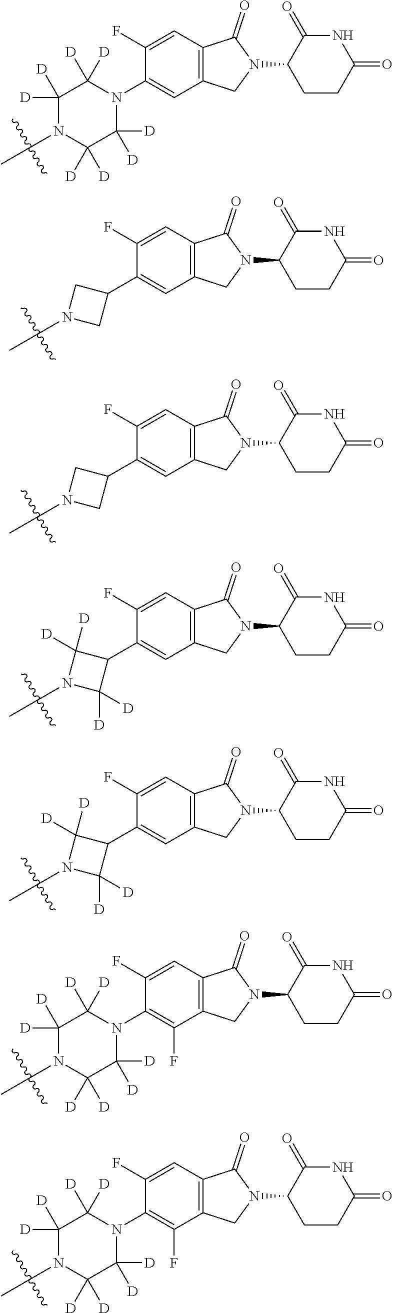

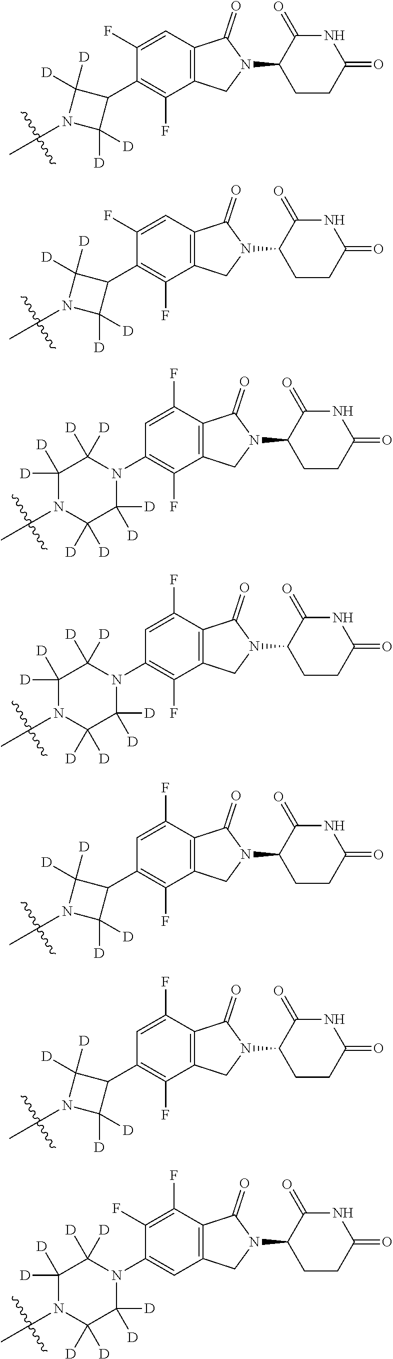

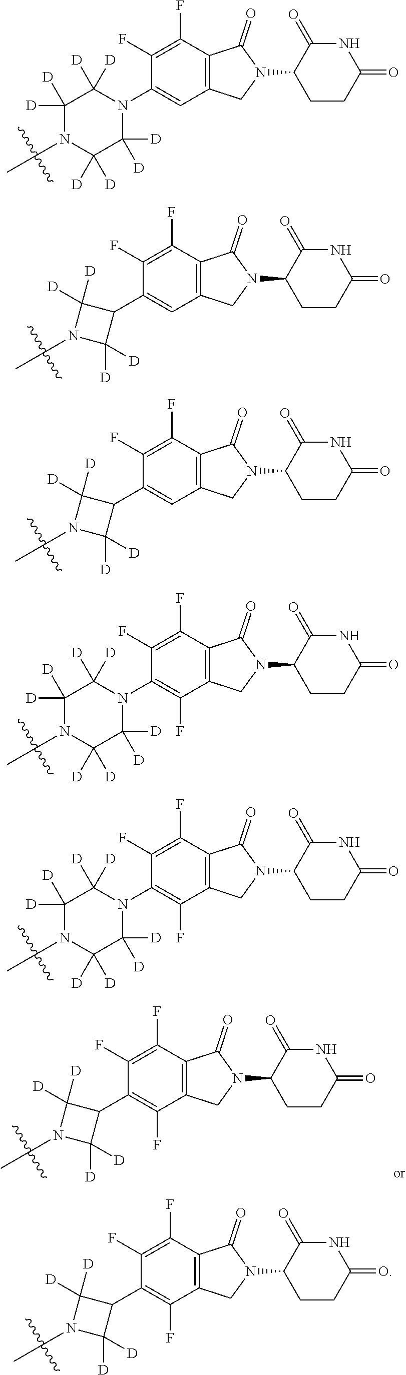

In some embodiments, LIN represents the structures of the following formula:
#-C₁₋₃₀ alkylene-;
#-(C(R^{a1})(R^{a2}))ₙ₁—(Rₑ(C(R^{a3})(R^{a4}))ₙ₂)ₘ₁—;
#-(C(R^{a1})(R^{a2}))ₙ₁—(Rₑ(C(R^{a3})(R^{a4}))ₙ₂)ₘ₁—(Rₑ(C(R^{a1})(R^{a6}))ₙ₃)ₘ₂—;
#-(C(R^{a1})(R^{a2}))ₙ₁—(Rₑ(C(R^{a3})(R^{a4}))ₙ₂)ₘ₁—(Rₑ(C(R^{a5})(R^{a6}))ₙ₃)ₘ₂—(Rₑ(C(R^{a7})(R^{a8}))ₙ₄)ₘ₃
#-(C(R^{a1})(R^{a2}))ₙ₁—(R_{f}(C(R^{a3})(R^{a4}))ₙ₂)ₘ₁—;
#-(C(R^{a1})(R^{a2}))ₙ₁—(R_{f}(C(R^{a3})(R^{a4}))ₙ₂)ₘ₁—(R_{f}(C(R^{a5})(R^{a6}))ₙ₃)ₘ₂—;
#-(C(R^{a1})(R^{a2}))ₙ₁—(R_{f}(C(R^{a3})(R^{a4}))ₙ₂)ₘ₁—(R_{f}(C(R^{a5})(R^{a6}))ₙ₃)ₘ₂—(R_{f}(C(R^{a7})(R^{a8}))ₙ₄)ₘ₃—;
#-(C(R^{a1})(R^{a2}))ₙ₁—(Rₑ—R_{f}—(C(R^{a3})(R^{a4}))ₙ₂)ₘ₁—;
#-(C(R^{a1})(R^{a2}))ₙ₁—(Rₑ(C(R^{a3})(R^{a4}))ₙ₂)ₘ₁—(R_{f}(C(R^{a5})(R^{a6}))ₙ₃)ₘ₂—;
#-(C(R^{a1})(R^{a2}))ₙ₁—(R_{f}—Rₑ—(C(R^{a3})(R^{a4}))ₙ₂)ₘ₁—; or
#-(C(R^{a1})(R^{a2}))ₙ₁—(R_{f}(C(R^{a3})(R^{a4}))ₙ₂)ₘ₁—(Rₑ(C(R^{a5})(R^{a6}))ₙ₃)ₘ₂—;
wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl (e.g., C₁₋₄ alkyl, such as methyl, ethyl, propyl, isopropyl, butyl, sec-butyl or tert-butyl); wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₃₋₆ cycloalkyl (e.g., optionally deuterated cyclopropyl, cyclobutyl, cyclopentyl, or cyclohexyl), hydroxy, amino, mercapto, halogen (e.g., fluorine, chlorine, bromine, or iodine), optionally deuterated C₁₋₆ alkoxy, optionally deuterated C₁₋₆ alkyl-NH—, halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), NH₂—C₁₋₆ alkylene, optionally deuterated C₁₋₆ alkyl-NHC(O)—, optionally deuterated C₁₋₆ alkyl-C(O)NH—, cyano, or any combination thereof;
a hydrogen atom of one or more CH₂ groups of the C₁₋₃₀ alkylene group is optionally replaced with a substituent selected from the group consisting of deuterium, hydroxy, amino, mercapto, nitro, halogen (e.g., fluorine, chlorine, bromine, or iodine), cyano, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally deuterated cyclopropyl, cyclobutyl, cyclopentyl, or cyclohexyl), optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH—, or any combination thereof;
R^{a1}, R^{a2}, R^{a3}, R^{a4}, R^{a5}, R^{a6}, R^{a7}, and R^{a8} each independently represent H, deuterium, hydroxy, amino, mercapto, nitro, halogen, cyano, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally deuterated cyclopropyl, cyclobutyl, cyclopentyl, or cyclohexyl), optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH—, or any combination thereof;
symbol # indicates the point of attachment to U₂; and
n1, n2, n3, n4, m1, m2, m3 each independently represent an integer of 1, 2, 3, 4, 5, 6, 7, 8, 9, 10, 11, 12, 13, 14, or 15.

In some embodiments, LIN represents the structures of the following formula:
#-(C(R^{a1})(R^{a2}))ₙ₁—(O(C(R^{a3})(R^{a4}))ₙ₂)ₘ₁—;
#-(C(R^{a1})(R^{a2}))ₙ₁—(O(C(R^{a3})(R^{a4}))ₙ₂)ₘ₁—(O(C(R^{a5})(R^{a6}))ₙ₃)ₘ₂—;
#-(C(R^{a1})(R^{a2}))ₙ₁—(O(C(R^{a3})(R^{a4}))ₙ₂)ₘ₁—(O(C(R^{a5})(R^{a6}))ₙ₃)ₘ₂—(O(C(R^{a7})(R^{a8}))ₙ₄)ₘ₃—;
#-(C(R^{a1})(R^{a2}))ₙ—(N(R_{g})(C(R^{a3})(R^{a4}))ₙ₂)ₘ₁—;
#-(C(R^{a1})(R^{a2}))ₙ—(N(R_{g})(C(R^{a3})(R^{a4}))ₙ₂)ₘ₁—(N(R_{g})(C(R^{a5})(R^{a6}))ₙ₃)ₘ₂—;
#-(C(R^{a1})(R^{a2}))ₙ—(N(R_{g})(C(R^{a3})(R^{a4}))ₙ₂)ₘ₁—(N(R_{g})(C(R^{a5})(R^{a6}))ₙ₃)ₘ₂—(N(R_{g})(C(R^{a7})(R^{a8}))ₙ₄)ₘ₃—;
#-(C(R^{a1})(R^{a2}))ₙ—(C(O)N(R_{g})—(C(R^{a3})(R^{a4}))ₙ₂)ₘ₁—;
#-(C(R^{a1})(R^{a2}))ₙ—(C(O)N(R_{g})—(C(R^{a3})(R^{a4}))ₙ₂)ₘ₁—(C(O)N(R_{g})—(C(R^{a5})(R^{a6}))ₙ₃)ₘ₂—;
#-(C(R^{a1})(R^{a2}))ₙ—(C(O)N(R_{g})—(C(R^{a3})(R^{a4}))ₙ₂)ₘ₁—(C(O)N(R_{g})—(C(R^{a5})(R^{a6}))ₙ₃)ₘ₂—(C(O)N(R_{g})—
(C(R^{a7})(R^{a8}))ₙ₄)ₘ₃—;
#-(C(R^{a1})(R^{a2}))ₙ—(C(O)N(R_{g})—(C(R^{a3})(R^{a4}))ₙ₂)ₘ₁—(O—(C(R^{a5})(R^{a6}))ₙ₃)ₘ₂—;
#-(C(R^{a1})(R^{a2}))ₙ₁—C(O)N(R_{g})—(C(R^{a3})(R^{a4}))ₙ₂—(O(C(R^{a5})(R^{a6}))ₙ₃)ₘ₁—;
#-(C(R^{a1})(R^{a2}))ₙ—(N(R_{g})C(O)—(C(R^{a3})(R^{a4}))ₙ₂)ₘ₁—;
#-(C(R^{a1})(R^{a2}))ₙ₁—(N(R_{g})C(O)—(C(R^{a3})(R^{a4}))ₙ₂)ₘ₁—(O—(C(R^{a7})(R^{a8}))ₙ₃)ₘ₂—;
#-(C(R^{a1})(R^{a2}))ₙ₁—(N(R_{g})C(O)—(C(R^{a3})(R^{a4}))ₙ₂)ₘ₁—(O—(C(R^{a5})(R^{a6}))ₙ₃)ₘ₂—(O—(C(R_{g})(R^{a8}))ₙ₄)ₘ₃—;
#-(C(R^{a1})(R^{a2}))ₙ₁—N(R_{g})C(O)—(C(R^{a3})(R^{a4}))ₙ₂—(O(C(R^{a5})(R^{a6}))ₙ₃)ₘ₁—;
#-(C(R^{a1})(R^{a2}))ₙ₁—N(R_{g})C(O)N(R_{g})—(C(R^{a3})(R^{a4}))ₙ₂—;
#-(C(R^{a1})(R^{a2}))ₙ₁—C(O)—(C(R^{a3})(R^{a4}))ₙ₂—;
#-(C(R^{a1})(R^{a2}))ₙ₁-(arylene-(C(R^{a3})(R^{a4}))ₙ₂)ₘ—;
#-(C(R^{a1})(R^{a2}))ₙ₁-(arylene-(C(R^{a3})(R^{a4}))ₙ₂)ₘ₁-arylene-(C(R^{a5})(R^{a6}))ₙ₃—;
#-(C(R^{a1})(R^{a2}))ₙ₁-(heterocyclylene-(C(R^{a3})(R^{a4}))ₙ₂)ₘ₁—;
#-(C(R^{a1})(R^{a2}))ₙ₁-(heterocyclylene-(C(R^{a3})(R^{a4}))ₙ₂)ₘ₁-(heterocyclylene-(C(R^{a5})(R^{a6}))ₙ₃)ₘ₂—;
#-(C(R^{a1})(R^{a2}))ₙ₁-(heteroarylene-(C(R^{a3})(R^{a4}))ₙ₂)ₘ₁—;
#-(C(R^{a1})(R^{a2}))ₙ₁-(heteroarylene-(C(R^{a3})(R^{a4}))ₙ₂)ₘ₁-(heteroarylene-(C(R^{a5})(R^{a6}))₃)ₘ₂—;
#-(C(R^{a1})(R^{a2}))ₙ₁-(cycloalkylene-(C(R^{a3})(R^{a4}))ₙ₂)ₘ₁—; or
#-(C(R^{a1})(R^{a2}))ₙ₁-(cycloalkylene-(C(R^{a3})(R^{a4}))ₙ₂)ₘ₁-(cycloalkylene-(C(R^{a5})(R^{a6}))ₙ₃)ₘ₂—;
wherein each R_{g} independently represent H or C₁₋₆ alkyl;
the cycloalkylene, arylene, heterocyclylene, and heteroarylene are each independently optionally substituted with a substituent selected from the group consisting of deuterium, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₃₋₆ cycloalkyl (e.g., optionally deuterated cyclopropyl, cyclobutyl, cyclopentyl, or cyclohexyl), hydroxy, amino, mercapto, halogen (e.g., fluorine, chlorine, bromine, or iodine), optionally deuterated C₁₋₆ alkoxy, optionally deuterated C₁₋₆ alkyl-NH—, halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), NH₂—C₁₋₆ alkylene, optionally deuterated C₁₋₆ alkyl-NHC(O)—, optionally deuterated C₁₋₆ alkyl-C(O)NH—, cyano, or any combination thereof;
R^{a1}, R^{a2}, R^{a3}, R^{a4}, R^{a5}, R^{a6}, R^{a7}, and R^{a8} each independently represent H, deuterium, hydroxy, amino, mercapto, nitro, halogen (e.g., fluorine, chlorine, bromine, or iodine), cyano, optionally deuterated C₁₋₆ alkyl (e.g., optionally deuterated C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), optionally deuterated C₃₋₆ cycloalkyl (e.g., optionally deuterated cyclopropyl, cyclobutyl, cyclopentyl, or cyclohexyl), optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH—, or any combination thereof;
symbol # indicates the point of attachment to U₂; and
n1, n2, n3, n4, m1, m2, m3 each independently represent an integer of 1, 2, 3, 4, 5, 6, 7, 8, 9, 10, 11, 12, 13, 14, or 15.

In some embodiments, LIN represents the following groups:
#-CH₂—, #-(CH₂)₂—, #-(CH₂)₃—, #-(CH₂)₄—, #-(CH₂)₅—, #-(CH₂)₆—, #-(CH₂)₇—, #- (CH₂)₈—, #-(CH₂)₉—, #-(CH₂)₁₀—, #-(CH₂)₁₁—, #-(CH₂)₁₂—, #-(CH₂)₁₃—, #-(CH₂)₁₄—, #-(CH₂)₁₅—, #-(CH₂)₁₆—, #-(CH₂)₁₇—, #-(CH₂)₁₈—, #-(CH₂)₁₉—, #-(CH₂)₂₀—, #-(CH₂)₂₁—, #-(CH₂)₂₂—, #-(CH₂)₂₅—, or #-(CH₂)₃₀—; #-CH₂—O—CH₂—, #-CH₂—O—(CH₂)₂—, #-(CH₂)₁—O—(CH₂)₃—, #-(CH₂)₁—O—(CH₂)₄—, #-(CH₂)₁—O—(CH₂)₅—, #-(CH₂)₁—O—(CH₂)₆—, #-(CH₂)₁—O—(CH₂)₇—, #-(CH₂)₁—O—(CH₂)₈—, #-(CH₂)₁—O—(CH₂)₉—, #-(CH₂)₁—O—(CH₂)₁₀—, #-(CH₂)₂—O—(CH₂)₁—, #-(CH₂)₂—O—(CH₂)₂—, #-(CH₂)₂—O—(CH₂)₃—, #-(CH₂)₂—O—(CH₂)₄—, #-(CH₂)₂—O—(CH₂)₅—, #-(CH₂)₂—O—(CH₂)₆—, #-(CH₂)₂—O—(CH₂)₇—, #-(CH₂)₂—O—(CH₂)₈—, #-(CH₂)₂—O—(CH₂)₉—, #-(CH₂)₂—O—(CH₂)₁₀—, #-(CH₂)₂—O—(CH₂)₁₁—, #-(CH₂)₂—O—(CH₂)₁₂—, #-(CH₂)₃—O—(CH₂)₁—, #-(CH₂)₃—O—(CH₂)₂—, #-(CH₂)₃—O—(CH₂)₃—, #-(CH₂)₃—O—(CH₂)₄—, #-(CH₂)₃—O—(CH₂)₅—, #-(CH₂)₃—O—(CH₂)₆—, #-(CH₂)₃—O—(CH₂)₇—, #-(CH₂)₄—O—(CH₂)₁—, #-(CH₂)₄—O—(CH₂)₂—, #-(CH₂)₄—O—(CH₂)₃—, #-(CH₂)₄—O—(CH₂)₄—, #-(CH₂)₄—O—(CH₂)₅—, #-(CH₂)₄—O—(CH₂)₆—, #-(CH₂)₅—O—(CH₂)₁—, #-(CH₂)₅—O—(CH₂)₂—, #-(CH₂)₅—O—(CH₂)₃—, #-(CH₂)₅—O—(CH₂)₄—, #-(CH₂)₅—O—(CH₂)₅—, #-(CH₂)₆—O—(CH₂)₁—, #-(CH₂)₆—O—(CH₂)₂—, #-(CH₂)₆—O—(CH₂)₃—, #-(CH₂)₆—O—(CH₂)₄—, #-(CH₂)₇—O—(CH₂)₁—, #-(CH₂)₇—O—(CH₂)₂—, #-(CH₂)₇—O—(CH₂)₃—, #-(CH₂)₈—O—(CH₂)₁—, #-(CH₂)₈—O—(CH₂)₂—, #-CH(CH₃)—O—(CH₂)₁—, #-CH(CH₃)—O—(CH₂)₂—, #-CH(CH₃)—O—(CH₂)₃—, #-CH(CH₃)—O—(CH₂)₄—, #-CH(CH₃)—O—(CH₂)₅—, #-CH(CH₃)—O—(CH₂)₆—, #-CH(CH₃)—O—(CH₂)₇—, #-CH(CH₃)—O—(CH₂)₈—, #-CH(CH₃)—O—(CH₂)₉—, #-CH(CH₃)—O—(CH₂)₁₀—, #-CH₂—(O(CH₂)₂)₂—, #-CH₂—(O(CH₂)₂)₃—, #-CH₂—(O(CH₂)₂)₄—, #-CH₂—(O(CH₂)₂)₅—, #-CH₂—(O(CH₂)₂)₆—, #-CH₂—(O(CH₂)₂)₇—, #-CH₂—(O(CH₂)₂)₈—, #-CH₂—(O(CH₂)₂)₉—, #-CH₂—(O(CH₂)₂)₁₀—, #-CH₂—(O(CH₂)₂)₁—OCH₂—, #-CH₂—(O(CH₂)₂)₂—OCH₂—, #-CH₂—(O(CH₂)₂)₃—OCH₂—, #-CH₂—(O(CH₂)₂)₄—OCH₂—, #-CH₂—(O(CH₂)₂)₅—OCH₂—, #-CH₂—(O(CH₂)₂)₆—OCH₂—, #-CH₂—(O(CH₂)₂)₇—OCH₂—, #-CH₂—(O(CH₂)₂)₈—OCH₂—, #-CH₂—(O(CH₂)₂)₉—OCH₂—, #-CH₂—(O(CH₂)₂)₁₀—OCH₂—, #-(CH₂)₂—(O(CH₂)₂)₂—, #-(CH₂)₂—(O(CH₂)₂)₃—, #-(CH₂)₂—(O(CH₂)₂)₄—, #-(CH₂)₂—(O(CH₂)₂)₅—, #-(CH₂)₂—(O(CH₂)₂)₆—, #-(CH₂)₂—(O(CH₂)₂)₇—, #-(CH₂)₂—(O(CH₂)₂)₈—, #-(CH₂)₂—(O(CH₂)₂)₉—, #-(CH₂)₂—(O(CH₂)₂)₁₀—, #-(CH₂)₃—(O(CH₂)₂)₂—, #-(CH₂)₃—(O(CH₂)₂)₃—, #-(CH₂)₃—(O(CH₂)₂)₄—, #-(CH₂)₃—(O(CH₂)₂)₅—, #-(CH₂)₃—(O(CH₂)₂)₆—, #-(CH₂)₃—(O(CH₂)₂)₇—, #-(CH₂)₃—(O(CH₂)₂)₈—, #-(CH₂)₃—(O(CH₂)₂)₉—, #-(CH₂)₃—(O(CH₂)₂)₁₀—, #-(CH₂)₄—(O(CH₂)₂)₂—, #-(CH₂)₄—(O(CH₂)₂)₃—, #-(CH₂)₄—(O(CH₂)₂)₄—, #-(CH₂)₄—(O(CH₂)₂)₅—, #-(CH₂)₄—(O(CH₂)₂)₆—, #-(CH₂)₄—(O(CH₂)₂)₇—, #-(CH₂)₄—(O(CH₂)₂)₈—, #-(CH₂)₄—(O(CH₂)₂)₉—, #-(CH₂)₄—(O(CH₂)₂)₁₀—, #-CH₂—(O(CH₂)₃)₂—, #-CH₂—(O(CH₂)₃)₃—, #-CH₂—(O(CH₂)₃)₄—, #-CH₂—(O(CH₂)₃)₅—, #-CH₂—(O(CH₂)₃)₆—, #-CH₂—(O(CH₂)₃)₇—, #-CH₂—(O(CH₂)₃)₈—, #-CH₂—(O(CH₂)₃)₉—, #- CH₂—(O(CH₂)₃)₁₀—, #-(CH₂)₂—(O(CH₂)₃)₂—, #-(CH₂)₂—(O(CH₂)₃)₃—, #-(CH₂)₂—(O(CH₂)₃)₄—, #-(CH₂)₂—(O(CH₂)₃)₅—, #-(CH₂)₂—(O(CH₂)₃)₆—, #-(CH₂)₂—(O(CH₂)₃)₇—, #-(CH₂)₂—(O(CH₂)₃)₈—, #-(CH₂)₂—(O(CH₂)₃)₉—, #-(CH₂)₂—(O(CH₂)₃)₁₀—, #-(CH₂)₃—(O(CH₂)₃)₂—, #-(CH₂)₃—(O(CH₂)₃)₃—, #-(CH₂)₃—(O(CH₂)₃)₄—, #-(CH₂)₃—(O(CH₂)₃)₅—, #-(CH₂)₃—(O(CH₂)₃)₆—, #-(CH₂)₃—(O(CH₂)₃)₇—, #-(CH₂)₃—(O(CH₂)₃)₈—, #-(CH₂)₃—(O(CH₂)₃)₉—, #-(CH₂)₃—(O(CH₂)₃)₁₀—, #-CH₂—O—(CH₂)₂—O—(CH₂)₃—, #-CH₂—(O(CH₂)₂)₂—(O(CH₂)₃)₂—, #-CH₂—(O(CH₂)₂)₃—(O(CH₂)₃)₃—, #-CH₂—(O(CH₂)₂)₄—(O(CH₂)₃)₄—, #-CH₂—(O(CH₂)₂)₅—(O(CH₂)₃)₅—, #-CH₂—(O(CH₂)₂)₆—(O(CH₂)₃)₆—, #-(CH₂)₂—O—(CH₂)₂—O—(CH₂)₃—, #-(CH₂)₂—(O(CH₂)₂)₂—(O(CH₂)₃)₂—, #-(CH₂)₂—(O(CH₂)₂)₃—(O(CH₂)₃)₃—, #-(CH₂)₂—(O(CH₂)₂)₄—(O(CH₂)₃)₄—, #-(CH₂)₂—(O(CH₂)₂)₅—(O(CH₂)₃)₅—, #-(CH₂)₂—(O(CH₂)₂)₆—(O(CH₂)₃)₆—, #-(CH₂)₃—O—(CH₂)₂—O—(CH₂)₃—, #-(CH₂)₃—(O(CH₂)₂)₂—(O(CH₂)₃)₂—, #-(CH₂)₃—(O(CH₂)₂)₃—(O(CH₂)₃)₃—, #-(CH₂)₃—(O(CH₂)₂)₄—(O(CH₂)₃)₄—, #-(CH₂)₃—(O(CH₂)₂)₅—(O(CH₂)₃)₅—, #-(CH₂)₃—(O(CH₂)₂)₆—(O(CH₂)₃)₆—, #-CH₂—O—(CH₂)₃—O—(CH₂)₂—, #-CH₂—(O(CH₂)₃)₂—(O(CH₂)₂)₂—, #-CH₂—(O(CH₂)₃)₃—(O(CH₂)₂)₃—, #-CH₂—(O(CH₂)₃)₄—(O(CH₂)₂)₄—, #-CH₂—(O(CH₂)₃)₅—(O(CH₂)₂)₅—, #-CH₂—(O(CH₂)₃)₆—(O(CH₂)₂)₆—, #-(CH₂)₂—O—(CH₂)₃—O—(CH₂)₂—, #-(CH₂)₂—(O(CH₂)₃)₂—(O(CH₂)₂)₂—, #-(CH₂)₂—(O(CH₂)₃)₃—(O(CH₂)₂)₃—, #-(CH₂)₂—(O(CH₂)₃)₄—(O(CH₂)₂)₄—, #-(CH₂)₂—(O(CH₂)₃)₅—(O(CH₂)₂)₅—, #-(CH₂)₂—(O(CH₂)₃)₆—(O(CH₂)₂)₆—, #-(CH₂)₃—O—(CH₂)₃—O—(CH₂)₂—, #-(CH₂)₃—(O(CH₂)₃)₂—(O(CH₂)₂)₂—, #-(CH₂)₃—(O(CH₂)₃)₃—(O(CH₂)₂)₃—, #-(CH₂)₃—(O(CH₂)₃)₄—(O(CH₂)₂)₄—, #-(CH₂)₃—(O(CH₂)₃)₅—(O(CH₂)₂)₅—, #-(CH₂)₃—(O(CH₂)₃)₆—(O(CH₂)₂)₆—, #-CH₂—O—(CH₂)₂O—CH₂—, #-(CH₂)₂—O—(CH₂)₂O—CH₂—, #-(CH₂)₂—(O(CH₂)₂)₂—O—(CH₂)₃—, #-(CH₂)₂—(O(CH₂)₂)₃—O—(CH₂)₃—, #-(CH₂)₂—(O(CH₂)₂)₄—O—(CH₂)₃—, #-(CH₂)₅—(O(CH₂)₂)₂—O—(CH₂)₅—, #-(CH₂)₅—(O(CH₂)₂)₂—O—(CH₂)₆—, #-(CH₂)₁—N(R_{g})—(CH₂)₁—, #-(CH₂)₁—N(R_{g})—(CH₂)₂—, #-(CH₂)₁—N(R_{g})—(CH₂)₃—, #-(CH₂)₁—N(R_{g})—(CH₂)₄—, #-(CH₂)₁—N(R_{g})—(CH₂)₅—, #-(CH₂)₁—N(R_{g})—(CH₂)₆—, #-(CH₂)₁—N(R_{g})—(CH₂)₇—, #-(CH₂)₁—N(R_{g})—(CH₂)₈—, #-(CH₂)₁—N(R_{g})—(CH₂)₉—, #-(CH₂)₁—N(R_{g})—(CH₂)₁₀—, #-(CH₂)₂—N(R_{g})—(CH₂)₁—, #-(CH₂)₂—N(R_{g})—(CH₂)₂—, #-(CH₂)₂—N(R_{g})—(CH₂)₃—, #-(CH₂)₂—N(R_{g})—(CH₂)₄—, #-(CH₂)₂—N(R_{g})—(CH₂)₅—, #-(CH₂)₂—N(R_{g})—(CH₂)₆—, #-(CH₂)₂—N(R_{g})—(CH₂)₇—, #-(CH₂)₂—N(R_{g})—(CH₂)₈—, #-(CH₂)₂—N(R_{g})—(CH₂)₉—, #-(CH₂)₂—N(R_{g})—(CH₂)₁₀—, #-(CH₂)₂—N(R_{g})—(CH₂)₁₁—, #-(CH₂)₂—N(R_{g})—(CH₂)₁₂—, #-(CH₂)₃—N(R_{g})—(CH₂)₁—, #-(CH₂)₃—N(R_{g})—(CH₂)₂—, #-(CH₂)₃—N(R_{g})—(CH₂)₃—, #-(CH₂)₄—N(R_{g})—(CH₂)₁—, #-(CH₂)₄—N(R_{g})—(CH₂)₂—, #-(CH₂)₄—N(R_{g})—(CH₂)₃—, #-(CH₂)₄—N(R_{g})—(CH₂)₄—, #-(CH₂)₅—N(R_{g})—(CH₂)₁—, #-(CH₂)₅—N(R_{g})—(CH₂)₂—, #-(CH₂)₅—N(R_{g})—(CH₂)₃—, #-(CH₂)₅—N(R_{g})—(CH₂)₄—, #-(CH₂)₅—N(R_{g})—(CH₂)₅—, #-(CH₂)₆—N(R_{g})—(CH₂)₁—, #-(CH₂)₆—N(R_{g})—(CH₂)₂—, #-(CH₂)₆—N(R_{g})—(CH₂)₃—, #-(CH₂)₇—N(R_{g})—(CH₂)₁—, #-(CH₂)₇—N(R_{g})—(CH₂)₂—, #-(CH₂)₇—N(R_{g})—(CH₂)₃—, #-(CH₂)₈—N(R_{g})—(CH₂)₁—, #-(CH₂)₈—N(R_{g})—(CH₂)₂—, #-(CH₂)₈—N(R_{g})—(CH₂)₃—, #-CH(CH₃)—N(R_{g})—(CH₂)₁—, #-CH(CH₃)—N(R_{g})—(CH₂)₂—, #-CH(CH₃)—N(R_{g})—(CH₂)₃—, #-CH(CH₃)—N(R_{g})—(CH₂)₄—, #-CH(CH₃)—N(R_{g})—(CH₂)₅—, #-CH(CH₃)—N(R_{g})—(CH₂)₆—, #-CH(CH₃)—N(R_{g})—(CH₂)₇—, #-CH(CH₃)—N(R_{g})—(CH₂)₈—, #-CH(CH₃)—N(R_{g})—(CH₂)₉—, #-CH(CH₃)—N(R_{g})—(CH₂)₁₀—, #-CH₂C(O)NHCH₂—, #-(CH₂)₂C(O)NH(CH₂)₂—, #-(CH₂)₂C(O)NH(CH₂)₃—, #-(CH₂)₂C(O)NH(CH₂)₄—, #-(CH₂)₂C(O)NH(CH₂)₅—, #-(CH₂)₃C(O)NH(CH₂)₃—, #-(CH₂)₃C(O)NH(CH₂)₄—, #-(CH₂)₄C(O)NH(CH₂)₄—, #-(CH₂)₅C(O)NH(CH₂)₅—, #-(CH₂)₆C(O)NH(CH₂)₇—, #-(CH₂)₆C(O)NH(CH₂)₆—, #-(CH₂)₇C(O)NH(CH₂)₇—, #-(CH₂)₈C(O)NH(CH₂)₈, U—(CH₂)₉C(O)NH(CH₂)₉—, #-(CH₂)₁₀C(O)NH(CH₂)₁₀—, #-(CH₂)₂C(O)NH(CH₂)₂—O—(CH₂)₂—, #-CH₂NHC(O)CH₂—, #-(CH₂)₂NHC(O)(CH₂)₂—, #-(CH₂)₂NHC(O)(CH₂)₃—, #-(CH₂)₂NHC(O)(CH₂)₄—, #-(CH₂)₂NHC(O)(CH₂)₅—, #-(CH₂)₃NHC(O)(CH₂)₃—, #-(CH₂)₃NHC(O)(CH₂)₄—, #-(CH₂)₄NHC(O)(CH₂)₄—, #-(CH₂)₅NHC(O)(CH₂)₅—, #-(CH₂)₆NHC(O)(CH₂)₇—, #-(CH₂)₆NHC(O)(CH₂)₆—, #-(CH₂)₇NHC(O)(CH₂)₇—, #-(CH₂)₈NHC(O)(CH₂)₈, #-(CH₂)₉NHC(O)(CH₂)₉—, #-(CH₂)₁₀NHC(O)(CH₂)₁₀—, #-(CH₂)₄NHC(O)(CH₂)₈—, #-(CH₂)₂NHC(O)(CH₂)₂—O—(CH₂)₂—, #-(CH₂)₄NHC(O)CH₂—, #-CH₂-piperidinylene-CH₂—, #-CH₂-piperidinylene-(CH₂)₂—, #-CH₂-piperidinylene-(CH₂)₃—, #-CH₂-piperidinylene-(CH₂)₄—, #-CH₂-piperidinylene-(CH₂)₅—, #-CH₂-piperidinylene-(CH₂)₆—, #-CH₂-piperidinylene-(CH₂)₇—, #-CH₂-piperidinylene-(CH₂)₈—, #-(CH₂)₂-piperidinylene-(CH₂)₁—, #-(CH₂)₂-piperidinylene-(CH₂)₂—, #-(CH₂)₂-piperidinylene-(CH₂)₃—, #-(CH₂)₂-piperidinylene-(CH₂)₄—, #-(CH₂)₂-piperidinylene-(CH₂)₅—, #-(CH₂)₂-piperidinylene-(CH₂)₆—, #-(CH₂)₂-piperidinylene-(CH₂)₇—, #-(CH₂)₂-piperidinylene-(CH₂)₈—, #-(CH₂)₃-piperidinylene-CH₂—, #-(CH₂)₃-piperidinylene-(CH₂)₂—, #-(CH₂)₃-piperidinylene-(CH₂)₃—, #-(CH₂)₃-piperidinylene-(CH₂)₄—, #-(CH₂)₃-piperidinylene-(CH₂)₅—, #-(CH₂)₃-piperidinylene-(CH₂)₆—, #-(CH₂)₃-piperidinylene-(CH₂)₇—, #-(CH₂)₃-piperidinylene-(CH₂)₈—, #-(CH₂)₄-piperidinylene-CH₂—, #-(CH₂)₄-piperidinylene-(CH₂)₂—, #-(CH₂)₄-piperidinylene-(CH₂)₃—, #-(CH₂)₄-piperidinylene-(CH₂)₄—, #-(CH₂)₄-piperidinylene-(CH₂)₅—, #-(CH₂)₄-piperidinylene-(CH₂)₆—, #-(CH₂)₄-piperidinylene-(CH₂)₇—, #-(CH₂)₄-piperidinylene-(CH₂)₈—, #-(CH₂)₅-piperidinylene-(CH₂)₁—, #-(CH₂)₅-piperidinylene-(CH₂)₂—, #-(CH₂)₅-piperidinylene-(CH₂)₃—, #-(CH₂)₅-piperidinylene-(CH₂)₄—, #-(CH₂)₅-piperidinylene-(CH₂)₅—, #-(CH₂)₅-piperidinylene-(CH₂)₆—, #-(CH₂)₅-piperidinylene-(CH₂)₇—, #-(CH₂)₅-piperidinylene-(CH₂)₈—, #-(CH₂)₆-piperidinylene-(CH₂)₁—, #-(CH₂)₆-piperidinylene-(CH₂)₂—, #-(CH₂)₆-piperidinylene-(CH₂)₃—, #-(CH₂)₆-piperidinylene-(CH₂)₄—, #-(CH₂)₆-piperidinylene-(CH₂)₅—, #-(CH₂)₆-piperidinylene-(CH₂)₆—, #-(CH₂)₆-piperidinylene-(CH₂)₇—, #-(CH₂)₆-piperidinylene-(CH₂)₈—, #-(CH₂)₇-piperidinylene-(CH₂)₁—, #-(CH₂)₇-piperidinylene-(CH₂)₂—, #-(CH₂)₇-piperidinylene-(CH₂)₃—, #-(CH₂)₇-piperidinylene-(CH₂)₄—, #-(CH₂)₇-piperidinylene-(CH₂)₈—, #-(CH₂)₈-piperidinylene-CH₂—, #-(CH₂)₈-piperidinylene-(CH₂)₂—, #-(CH₂)₈-piperidinylene-(CH₂)₃—, #-(CH₂)₈-piperidinylene-(CH₂)₄—, #-(CH₂)₈-piperidinylene-(CH₂)₅—, #-(CH₂)₈-piperidinylene-(CH₂)₆—, #-(CH₂)₈-piperidinylene-(CH₂)₇—, #-(CH₂)₈-piperidinylene-(CH₂)₈—, #-CH₂—N(R_{g})—CH₂-piperidinylene-CH₂—, #-CH₂—N(R_{g})—CH₂-piperidinylene-(CH₂)₂—, #-CH₂—N(R_{g})—CH₂-piperidinylene-(CH₂)₃—, #-CH₂—N(R_{g})—CH₂-piperidinylene-(CH₂)₄—, #-CH₂—N(R_{g})—CH₂-piperidinylene-(CH₂)₅—, #-CH₂—N(R_{g})—CH₂-piperidinylene-(CH₂)₆—, #-CH₂—N(R_{g})—CH₂-piperidinylene-(CH₂)₇—, #-CH₂—N(R_{g})—CH₂-piperidinylene-(CH₂)₈—, #-CH₂—N(R_{g})—(CH₂)₂-piperidinylene-CH₂—, #-CH₂—N(R_{g})—(CH₂)₂-piperidinylene-(CH₂)₂—, #-CH₂—N(R_{g})—(CH₂)₂-piperidinylene-(CH₂)₃—, #-CH₂—N(R_{g})—(CH₂)₂-piperidinylene-(CH₂)₄—, #-CH₂—N(R_{g})—(CH₂)₂-piperidinylene-(CH₂)₅—, #-CH₂—N(R_{g})—(CH₂)₂-piperidinylene-(CH₂)₆—, #-CH₂—N(R_{g})—(CH₂)₂-piperidinylene-(CH₂)₇—, #-CH₂—N(R_{g})—(CH₂)₂-piperidinylene-(CH₂)₈—, #-(CH₂)₂—N(R_{g})—CH₂-piperidinylene-CH₂—, #-(CH₂)₂—N(R_{g})—CH₂-piperidinylene-(CH₂)₂—, #-(CH₂)₂—N(R_{g})—CH₂-piperidinylene-(CH₂)₃—, #-(CH₂)₂—N(R_{g})—CH₂-piperidinylene-(CH₂)₄—, #-(CH₂)₂—N(R_{g})—CH₂-piperidinylene-(CH₂)₅—, #-(CH₂)₂—N(R_{g})—CH₂-piperidinylene-(CH₂)₆—, #-(CH₂)₂—N(R_{g})—CH₂-piperidinylene-(CH₂)₇—, #-(CH₂)₂—N(R_{g})—CH₂-piperidinylene-(CH₂)₈—, #-(CH₂)₃—N(R_{g})—CH₂-piperidinylene-CH₂—, #-(CH₂)₃—N(R_{g})—CH₂-piperidinylene-(CH₂)₂—, #-(CH₂)₃—N(R_{g})—CH₂-piperidinylene-(CH₂)₃—, #-(CH₂)₃—N(R_{g})—CH₂-piperidinylene-(CH₂)₈—, #-(CH₂)₄—N(R_{g})—CH₂-piperidinylene-CH₂—, #-(CH₂)₄—N(R_{g})—CH₂-piperidinylene-(CH₂)₂—, #-(CH₂)₄—N(R_{g})—CH₂-piperidinylene-(CH₂)₃—, #-(CH₂)₄—N(R_{g})—CH₂-piperidinylene-(CH₂)₈—, #-(CH₂)₅—N(R_{g})—CH₂-piperidinylene-(CH₂)₃—, #-(CH₂)₅—N(R_{g})—CH₂-piperidinylene-(CH₂)₈—, #-(CH₂)₆—N(R_{g})—CH₂-piperidinylene-(CH₂)₃—, #-(CH₂)₆—N(R_{g})—CH₂-piperidinylene-(CH₂)₈—, #-(CH₂)₇—N(R_{g})—CH₂-piperidinylene-(CH₂)₃—, #-(CH₂)₇—N(R_{g})—CH₂-piperidinylene-(CH₂)₈—, #-(CH₂)₈—N(R_{g})—CH₂-piperidinylene-CH₂—, #-(CH₂)₈—N(R_{g})—CH₂-piperidinylene-(CH₂)₂—, #-(CH₂)₈—N(R_{g})—CH₂-piperidinylene-(CH₂)₃—, #-(CH₂)₈—N(R_{g})—CH₂-piperidinylene-(CH₂)₄—, #-(CH₂)₈—N(R_{g})—CH₂-piperidinylene-(CH₂)₅—, #-(CH₂)₈—N(R_{g})—CH₂-piperidinylene-(CH₂)₆—, #-(CH₂)₈—N(R_{g})—CH₂-piperidinylene-(CH₂)₇—, #-(CH₂)₈—N(R_{g})—CH₂-piperidinylene-(CH₂)₈—, #-CH₂-piperazinylene-CH₂—, #-CH₂-piperazinylene-(CH₂)₂—, #-CH₂-piperazinylene-(CH₂)₃—, #-CH₂-piperazinylene-(CH₂)₄—, #-CH₂-piperazinylene-(CH₂)₅—, #-CH₂-piperazinylene-(CH₂)₆—, #-CH₂-piperazinylene-(CH₂)₇—, #-CH₂-piperazinylene-(CH₂)₈—, #-(CH₂)₂-piperazinylene-(CH₂)₁—, #-(CH₂)₂-piperazinylene-(CH₂)₂—, #-(CH₂)₂-piperazinylene-(CH₂)₃—, #-(CH₂)₂-piperazinylene-(CH₂)₄—, #-(CH₂)₂-piperazinylene-(CH₂)₅—, #-(CH₂)₂-piperazinylene-(CH₂)₆—, #-(CH₂)₂-piperazinylene-(CH₂)₇—, #-(CH₂)₂-piperazinylene-(CH₂)₈—, #-(CH₂)₃-piperazinylene-CH₂—, #-(CH₂)₃-piperazinylene-(CH₂)₂—, #-(CH₂)₃-piperazinylene-(CH₂)₃—, #-(CH₂)₃-piperazinylene-(CH₂)₄—, #-(CH₂)₃-piperazinylene-(CH₂)₅—, #-(CH₂)₃-piperazinylene-(CH₂)₆—, #-(CH₂)₃-piperazinylene-(CH₂)₇—, #-(CH₂)₃-piperazinylene-(CH₂)₈—, #-(CH₂)₄-piperazinylene-CH₂—, #-(CH₂)₄-piperazinylene-(CH₂)₂—, #-(CH₂)₄-piperazinylene-(CH₂)₃—, #-(CH₂)₄-piperazinylene-(CH₂)₄—, #-(CH₂)₄-piperazinylene-(CH₂)₅—, #-(CH₂)₄-piperazinylene-(CH₂)₆—, #-(CH₂)₄-piperazinylene-(CH₂)₇—, #-(CH₂)₄-piperazinylene-(CH₂)₈—, #-(CH₂)₅-piperazinylene-(CH₂)₁—, #-(CH₂)₅-piperazinylene-(CH₂)₂—, #-(CH₂)₅-piperazinylene-(CH₂)₃—, #-(CH₂)₅-piperazinylene-(CH₂)₄—, #-(CH₂)₅-piperazinylene-(CH₂)₅—, #-(CH₂)₅-piperazinylene-(CH₂)₆—, #-(CH₂)₅-piperazinylene-(CH₂)₇—, #-(CH₂)₅-piperazinylene-(CH₂)₈—, #-(CH₂)₆-piperazinylene-(CH₂)₁—, #-(CH₂)₆-piperazinylene-(CH₂)₂—, #-(CH₂)₆-piperazinylene-(CH₂)₃—, #-(CH₂)₆-piperazinylene-(CH₂)₄—, #-(CH₂)₆-piperazinylene-(CH₂)₅—, #-(CH₂)₆-piperazinylene-(CH₂)₆—, #-(CH₂)₆-piperazinylene-(CH₂)₇—, #-(CH₂)₆-piperazinylene-(CH₂)₈—, #-(CH₂)₇-piperazinylene-(CH₂)₁—, #-(CH₂)₇-piperazinylene-(CH₂)₂—, #-(CH₂)₇-piperazinylene-(CH₂)₃—, #-(CH₂)₇-piperazinylene-(CH₂)₄—, #-(CH₂)₇-piperazinylene-(CH₂)₈—, #-(CH₂)₈-piperazinylene-CH₂—, #-(CH₂)₈-piperazinylene-(CH₂)₂—, #-(CH₂)₈-piperazinylene-(CH₂)₃—, #-(CH₂)₈-piperazinylene-(CH₂)₄—, #-(CH₂)₈-piperazinylene-(CH₂)₅—, #-(CH₂)₈-piperazinylene-(CH₂)₆—, #-(CH₂)₈-piperazinylene-(CH₂)₇—, or #-(CH₂)₈-piperazinylene-(CH₂)₈—;
wherein the piperidinylene and piperazinylene are each independently optionally substituted with a substituent selected from the group consisting of deuterium, optionally deuterated C₁₋₆ alkyl (e.g., optionally deuterated C₁₋₄ alkyl, such as methyl, CD₃, ethyl, propyl, isopropyl, butyl, sec-butyl or tert-butyl), optionally deuterated C₃₋₆ cycloalkyl (e.g., optionally deuterated cyclopropyl, cyclobutyl, cyclopentyl, or cyclohexyl), hydroxy, amino, mercapto, halogen (e.g., fluorine, chlorine, bromine, or iodine), optionally deuterated C₁₋₆ alkoxy (e.g., optionally deuterated C₁₋₄ alkoxy, such as methoxy, CD₃-O—, ethoxy, propoxy, isopropoxy, butoxy, sec-butoxy or tert-butoxy), optionally deuterated C₁₋₆ alkyl-NH—, halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), NH₂—C₁₋₆ alkylene, optionally deuterated C₁₋₆ alkyl-NHC(O)—, optionally deuterated C₁₋₆ alkyl-C(O)NH—, cyano, or any combination thereof;
each R_{g} independently represent H or C₁₋₆ alkyl;
symbol # indicates the point of attachment to U₂; and
n1, n2, n3, n4, m1, m2, m3 each independently represent an integer of 1, 2, 3, 4, 5, 6, 7, 8, 9, 10, 11, 12, 13, 14, or 15.

In some embodiments, —W¹-LIN—U₂—R_{c}—U₁— represents the following groups:

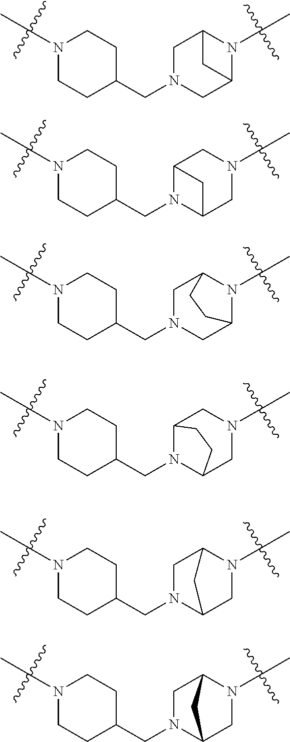 [SMILES: *N1CCC(CN2CC3CC(C2)N3*)CC1 ; *N1CCC(CN2C3CC2CN(*)C3)CC1 ; *N1CCC(CN2CC3CCC(C2)N3*)CC1 ; *N1CCC(CN2C3CCC2CN(*)C3)CC1 ; *N1CCC(CN2CC3CC2CN3*)CC1 ; *N1CCC(CN2C[C@H]3C[C@@H]2CN3*)CC1]

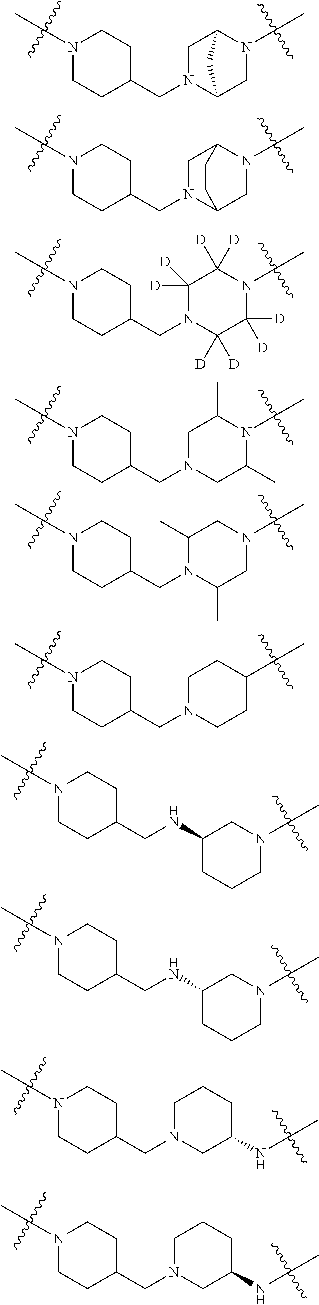

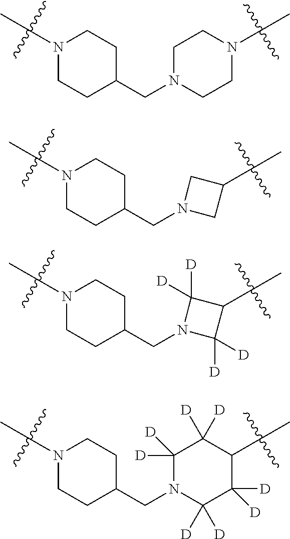

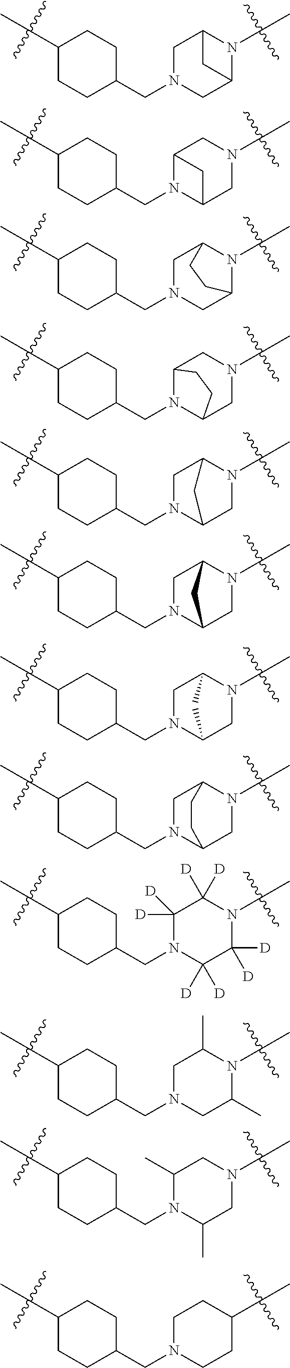

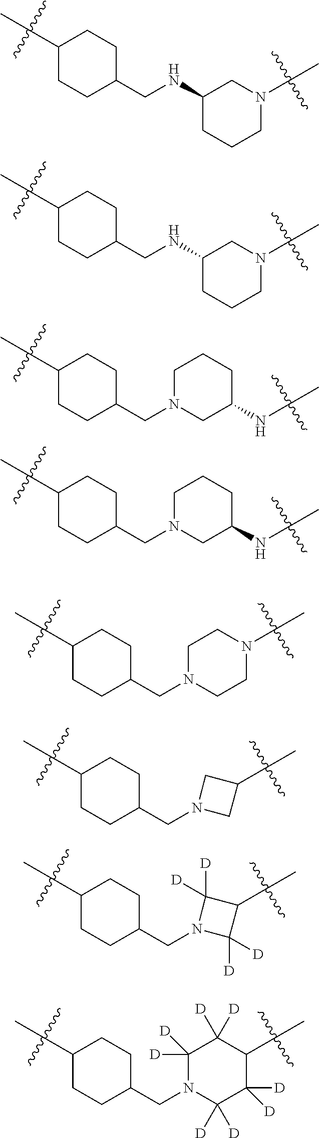

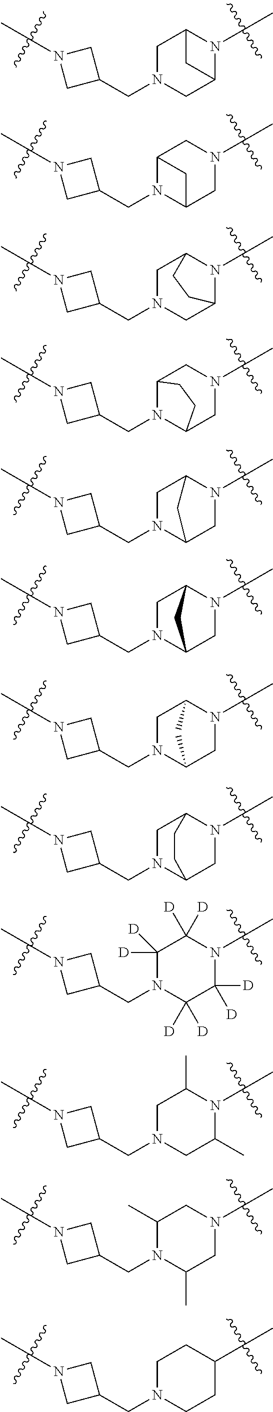

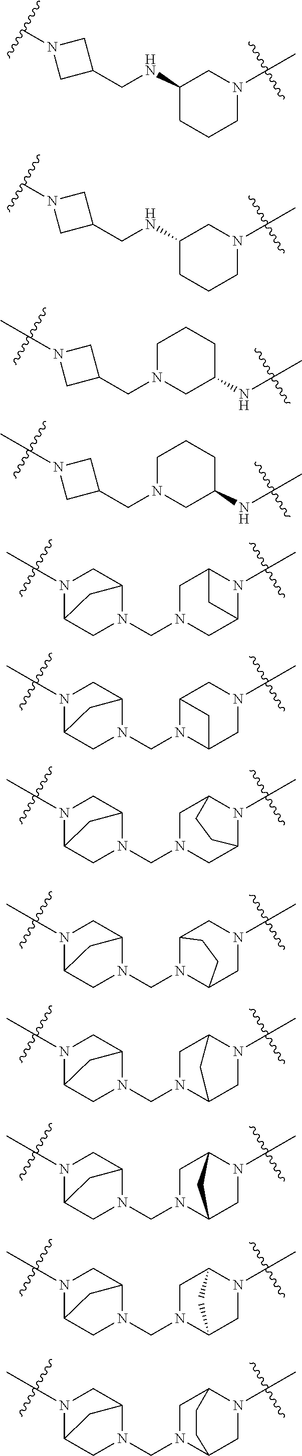

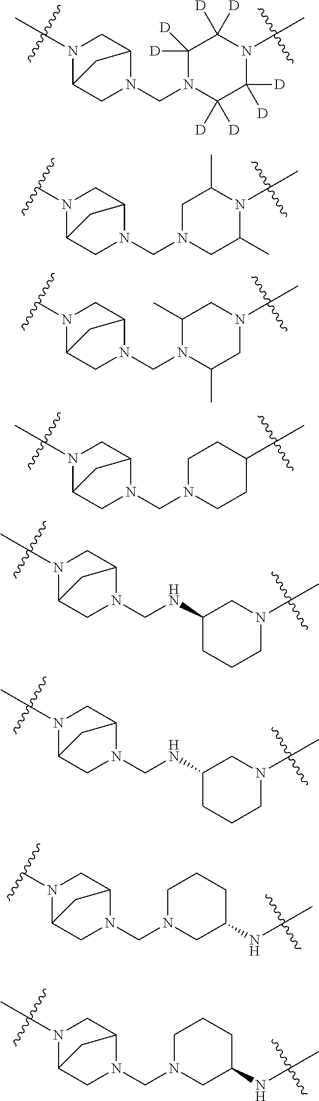

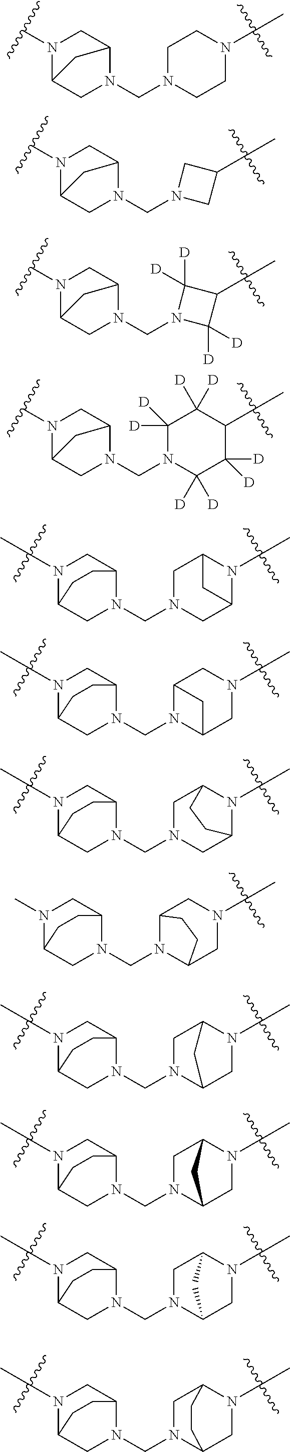

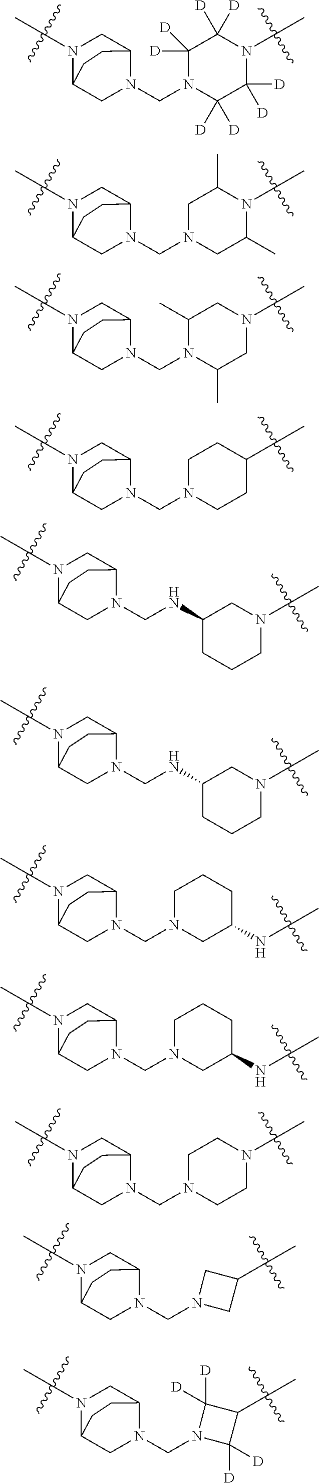

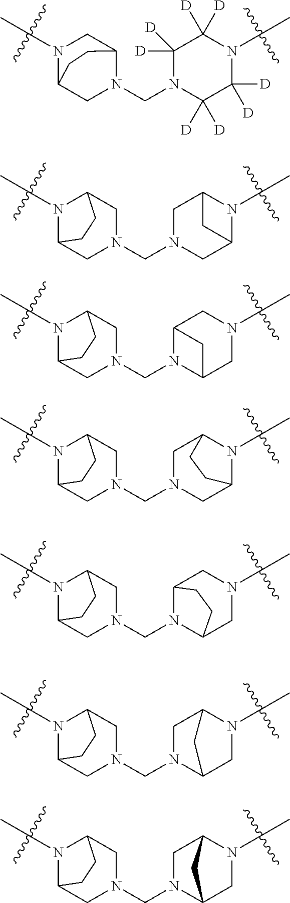

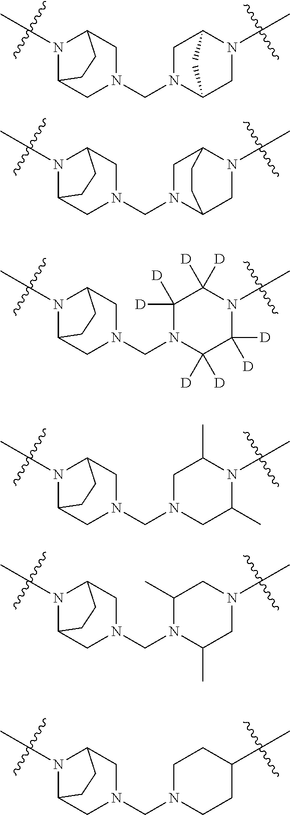

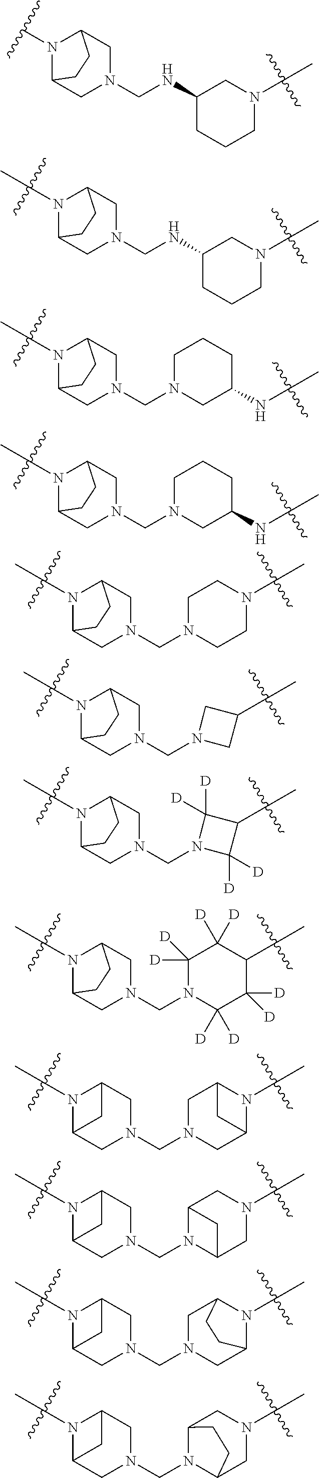

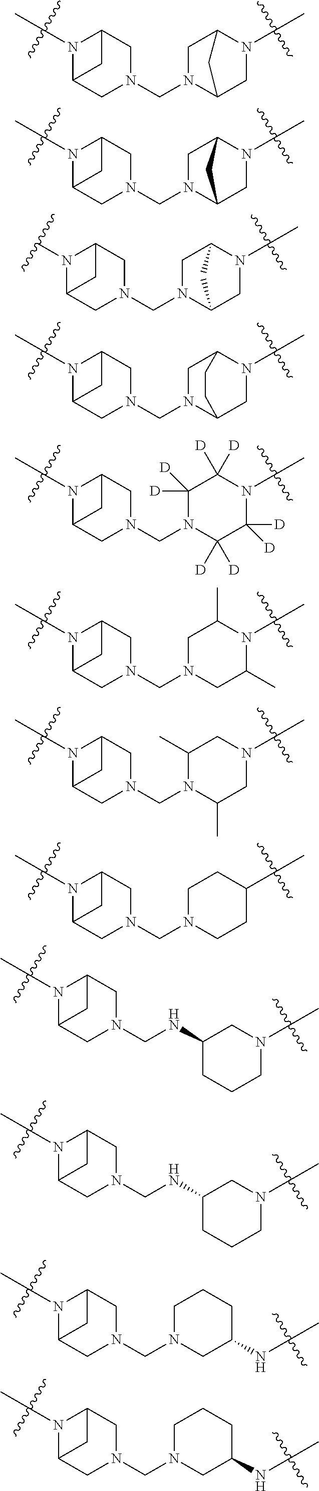

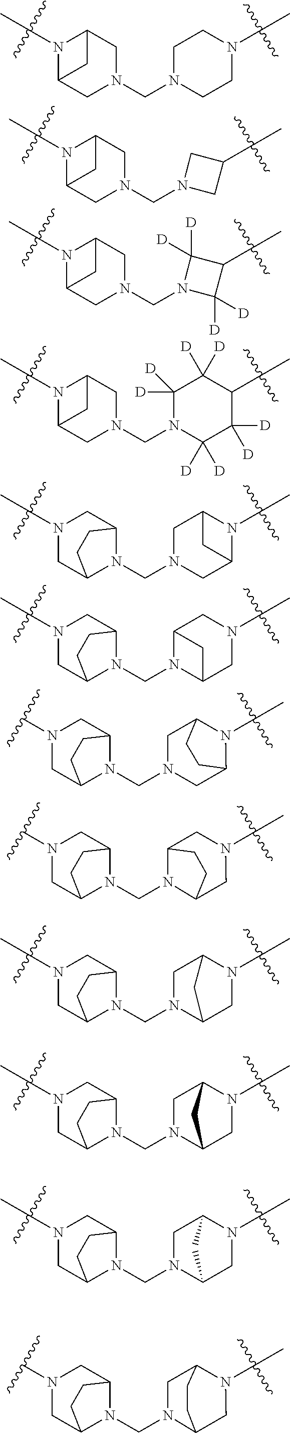

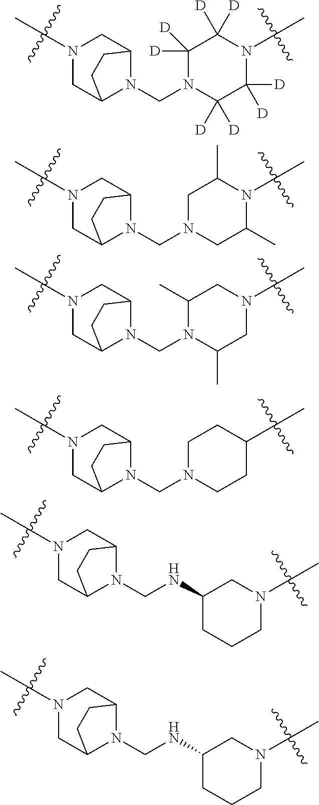

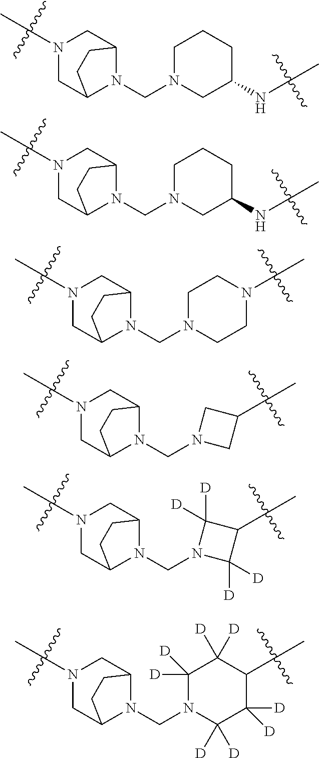

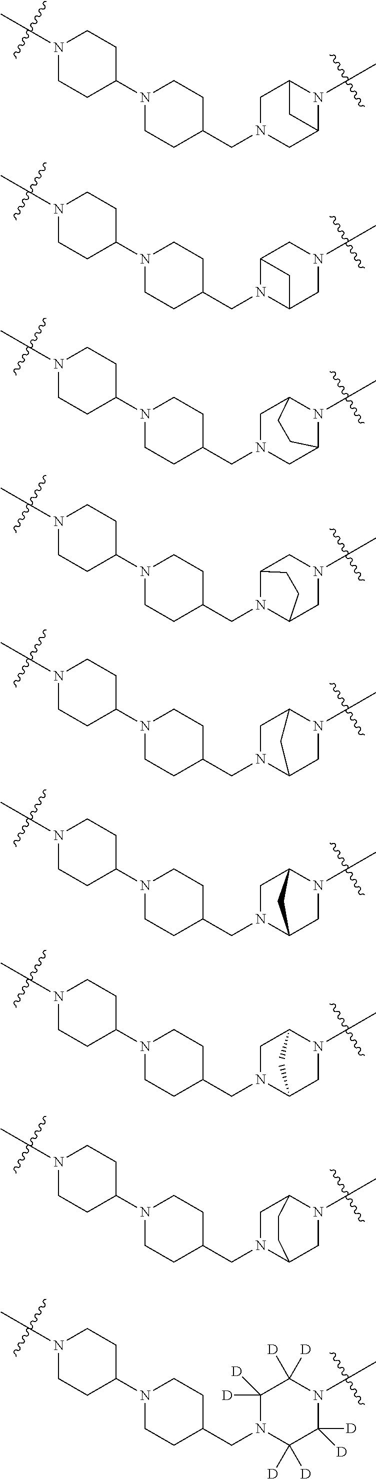

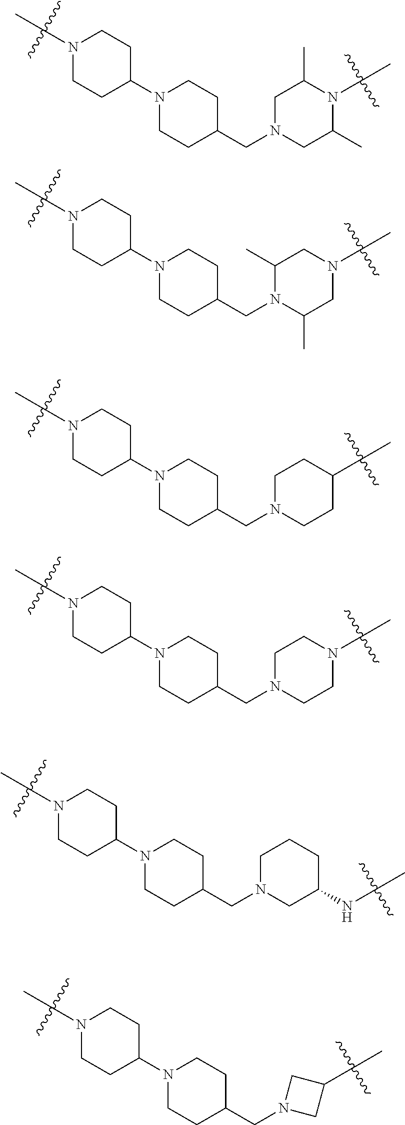 [SMILES: *N1CCC(N2CCC(CN3CC(C)N(*)C(C)C3)CC2)CC1 ; *N1CCC(N2CCC(CN3C(C)CN(*)CC3C)CC2)CC1 ; *C1CCN(CC2CCN(C3CCN(*)CC3)CC2)CC1 ; *N1CCC(N2CCC(CN3CCN(*)CC3)CC2)CC1 ; *N[C@H]1CCCN(CC2CCN(C3CCN(*)CC3)CC2)C1 ; *C1CN(CC2CCN(C3CCN(*)CC3)CC2)C1]

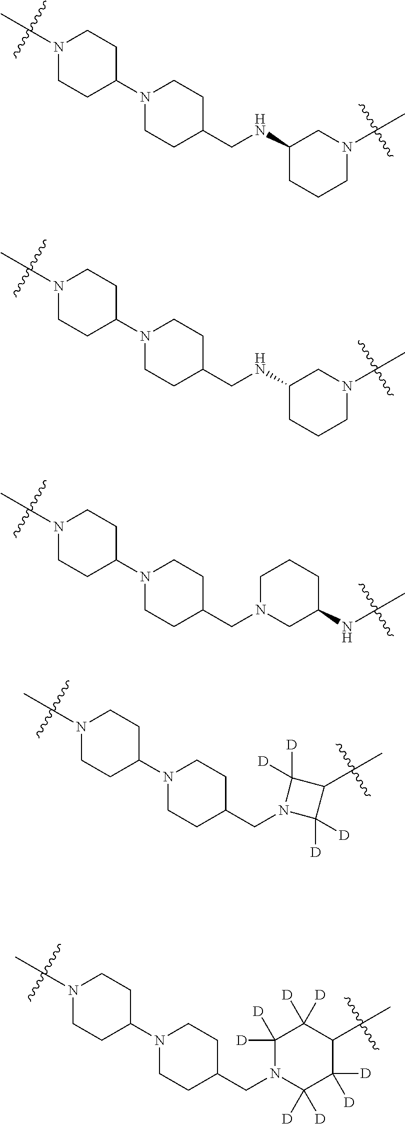 [SMILES: *N1CCC(N2CCC(CN[C@@H]3CCCN(*)C3)CC2)CC1 ; *N1CCC(N2CCC(CN[C@H]3CCCN(*)C3)CC2)CC1 ; *N[C@@H]1CCCN(CC2CCN(C3CCN(*)CC3)CC2)C1 ; *C1C([2H])([2H])N(CC2CCN(C3CCN(*)CC3)CC2)C1([2H])[2H] ; *C1C([2H])([2H])C([2H])([2H])N(CC2CCN(C3CCN(*)CC3)CC2)C([2H])([2H])C1([2H])[2H]]

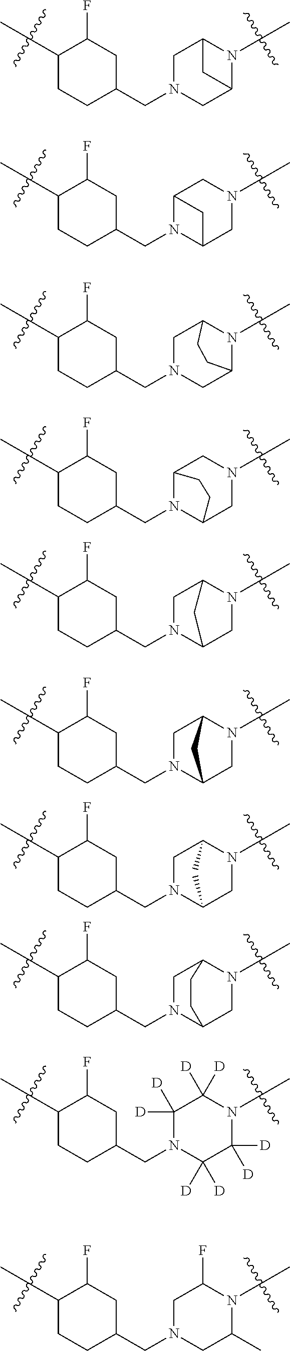

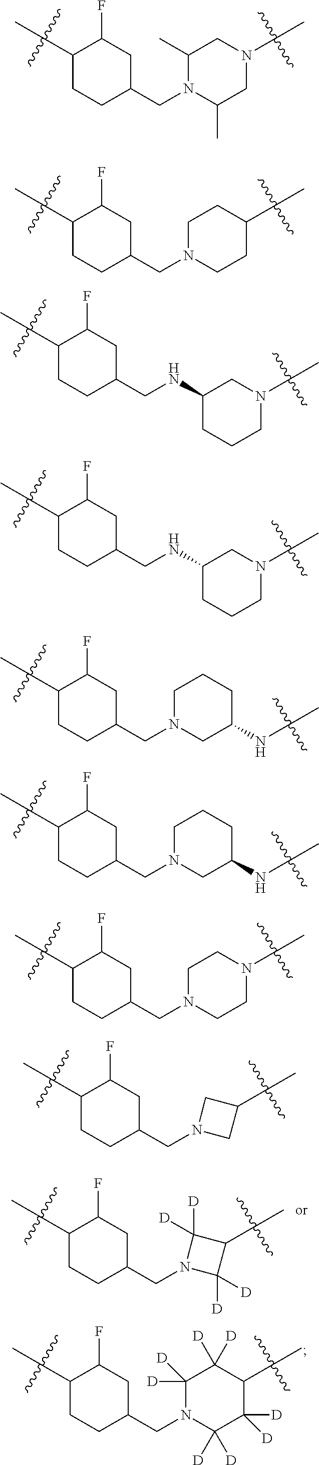

wherein the groups are optionally substituted with one or more (e.g., 1-10, 1-8, 1-6, such as 1, 2, 3, 4, 5 or 6) substituents selected from the group consisting of deuterium, halogen (e.g., fluorine, chlorine, bromine, or iodine), cyano, amino, C₁₋₆ alkyl (e.g., C₁₋₄ alkyl, such as methyl, CD₃, ethyl, propyl, isopropyl, butyl, sec-butyl or tert-butyl), halogenated C₁₋₆ alkyl (e.g., halogenated C₁₋₄ alkyl, such as F₃C—, FCH₂—, F₂CH—, ClCH₂—, Cl₂CH—, CF₃CF₂—, CF₃CHF—, CHF₂CF₂—, CHF₂CHF—, CF₃CH₂— or CH₂ClCH₂—), deuterated C₁₋₆ alkyl (e.g., perdeuterated C₁₋₄ alkyl, such as perdeuterated methyl (CD₃), perdeuterated ethyl, perdeuterated propyl, perdeuterated isopropyl, perdeuterated butyl, perdeuterated sec-butyl or perdeuterated tert-butyl), C₁₋₆ alkoxy (e.g., C₁₋₄ alkoxy, such as methoxy, ethoxy, propoxy, isopropoxy, butoxy, sec-butoxy or tert-butoxy), halogenated C₁₋₆ alkoxy (e.g., halogenated C₁₋₄ alkoxy, such as F₃C—O—, FCH₂—O—, F₂CH—O—, ClCH₂—O—, Cl₂CH—O—, CF₃CF₂—O—, CF₃CHF—O—, CHF₂CF₂—O—, CHF₂CHF—O—, CF₃CH₂—O— or CH₂ClCH₂—O—), deuterated C₁₋₆ alkoxy (e.g., perdeuterated C₁₋₄ alkoxy, such as D₃C—O—, CD₃CD₂-O—, or CD₃CD₂CD₂-O—), or any combination thereof.

In some embodiments, the compound of Formula (III) is also of Formula (III-1), Formula (III-2), Formula (III-3), Formula (III-4), Formula (III-5), Formula (III-6), Formula (III-7), Formula (III-8), Formula (III-9), Formula (III-10), Formula (III-11), Formula (III-12), Formula (III-13), Formula (III-14), Formula (III-15), Formula (III-16), Formula (III-17), Formula (III-18), Formula (III-19), Formula (III-20), Formula (III-21), Formula (III-22), Formula (III-23) or Formula (III-24):

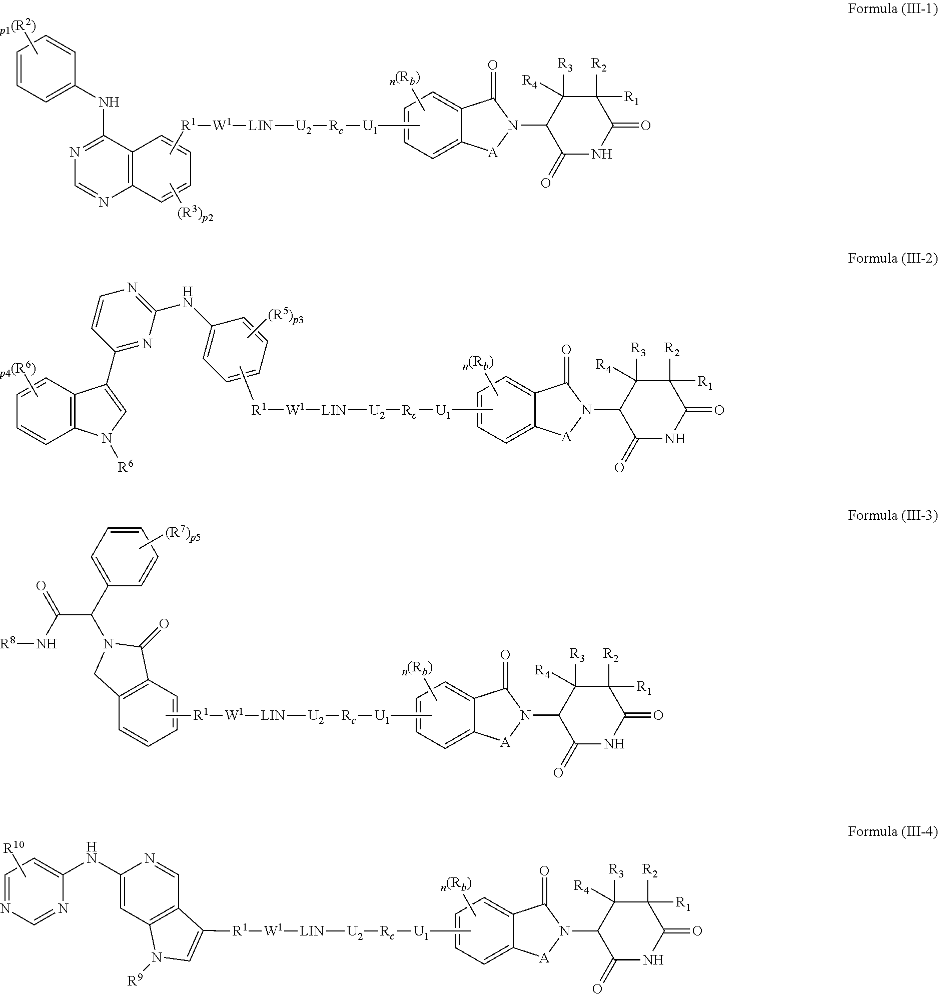

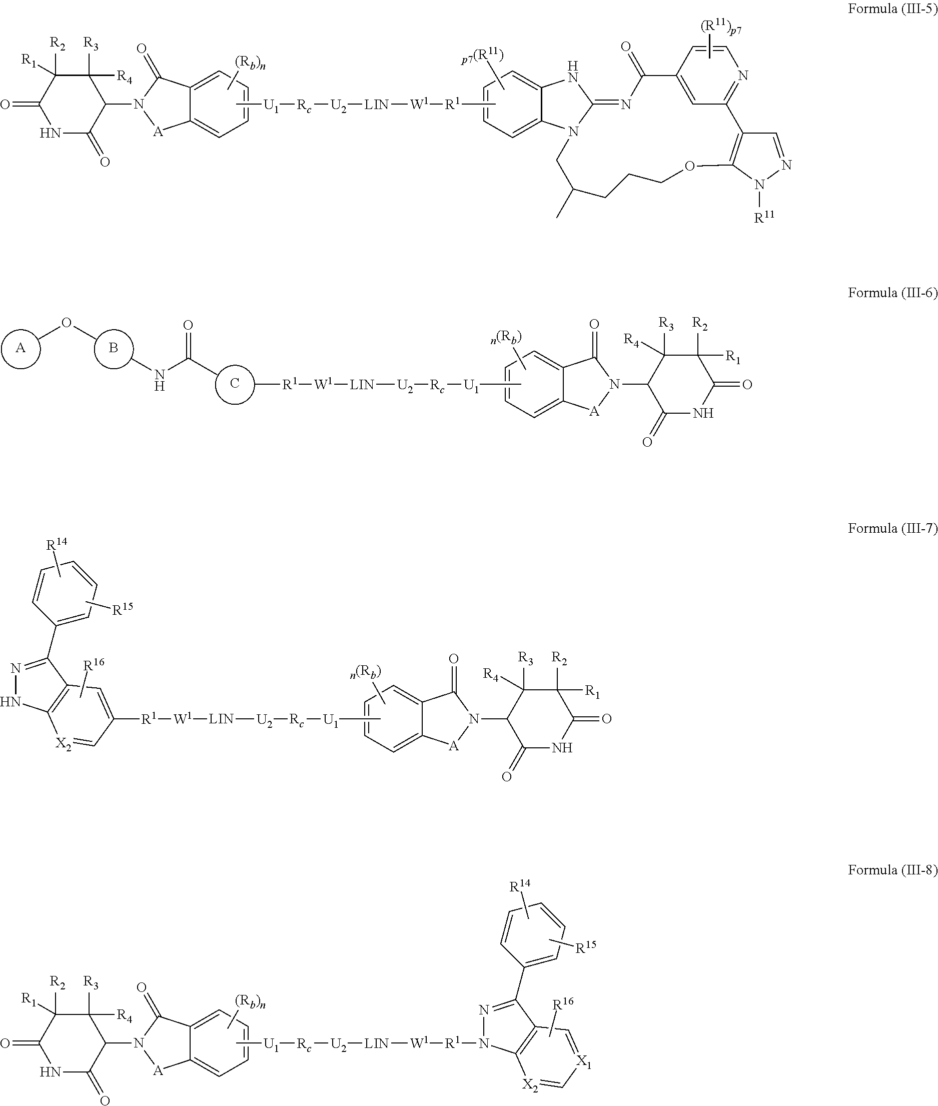

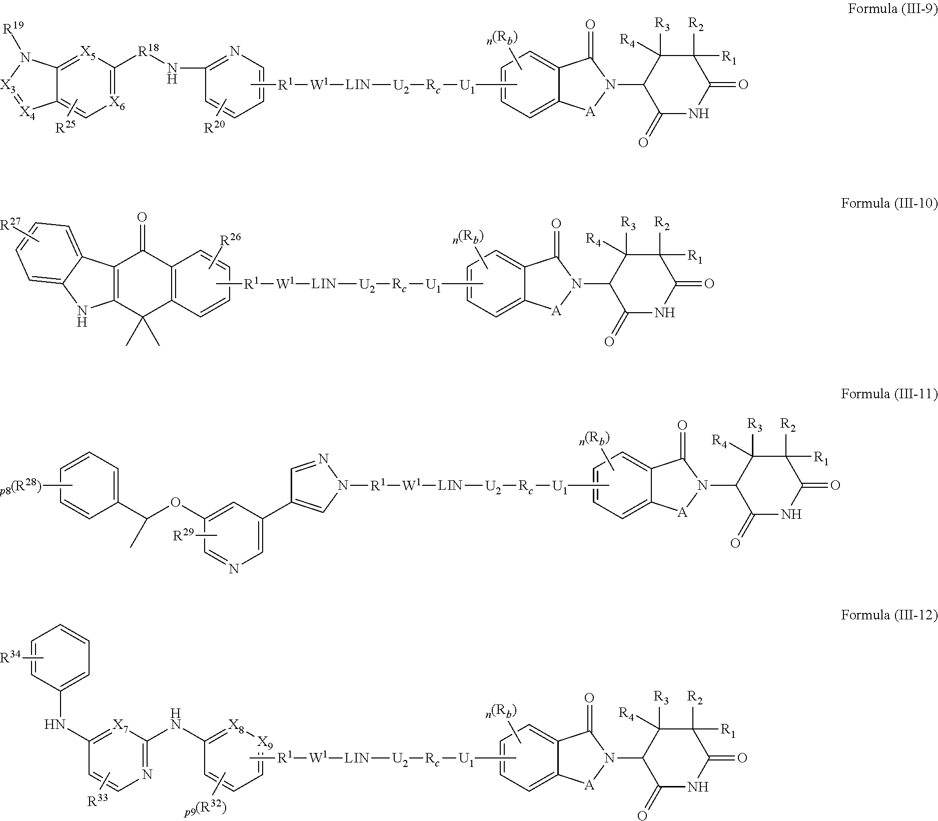

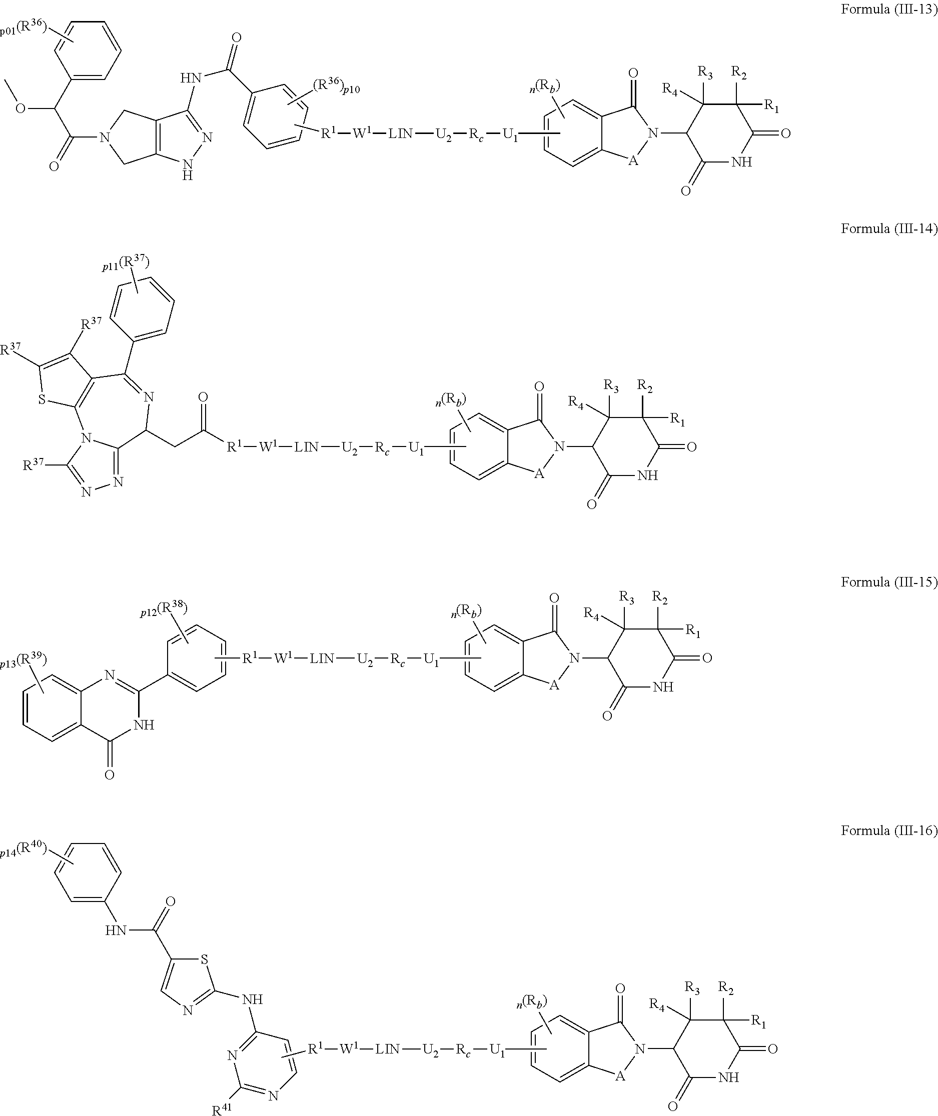

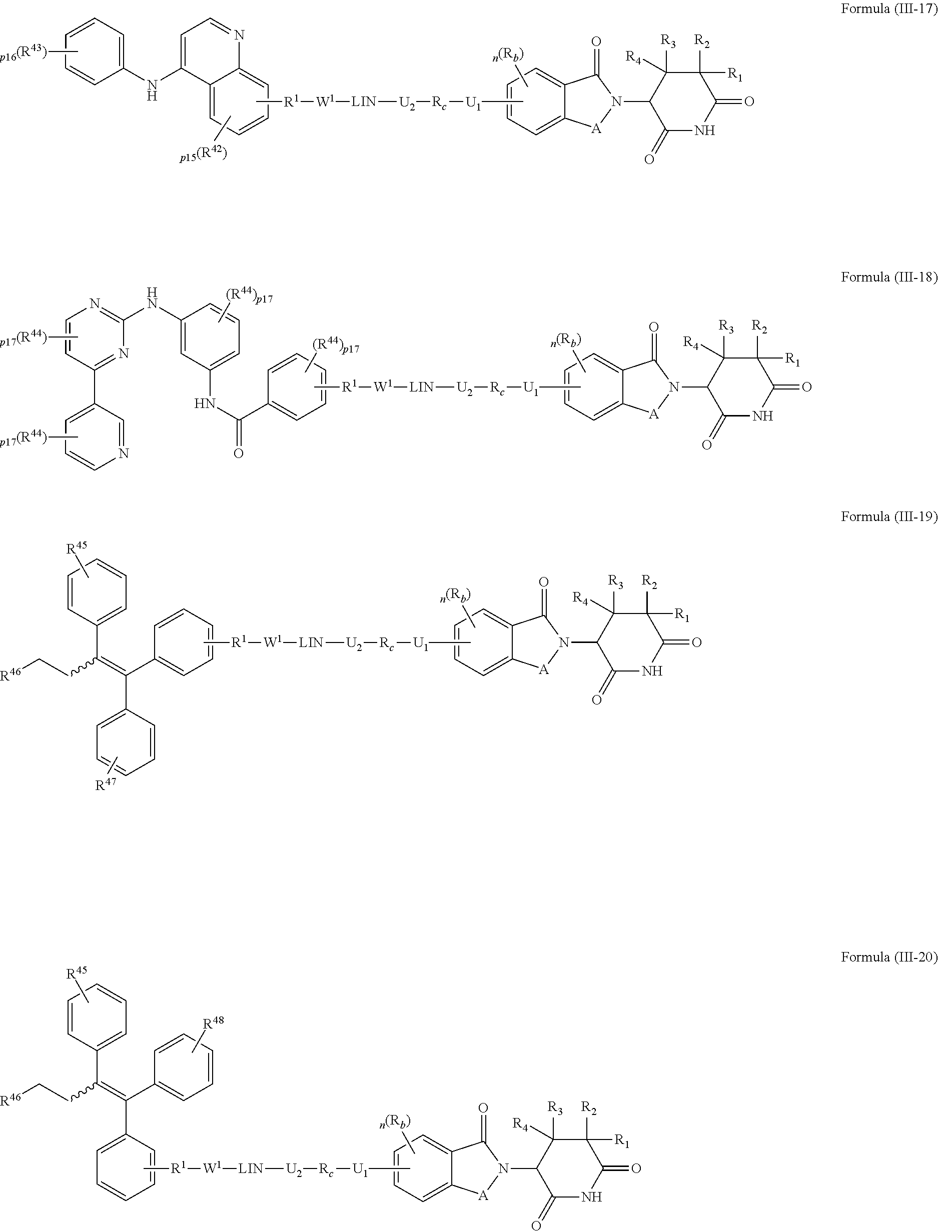

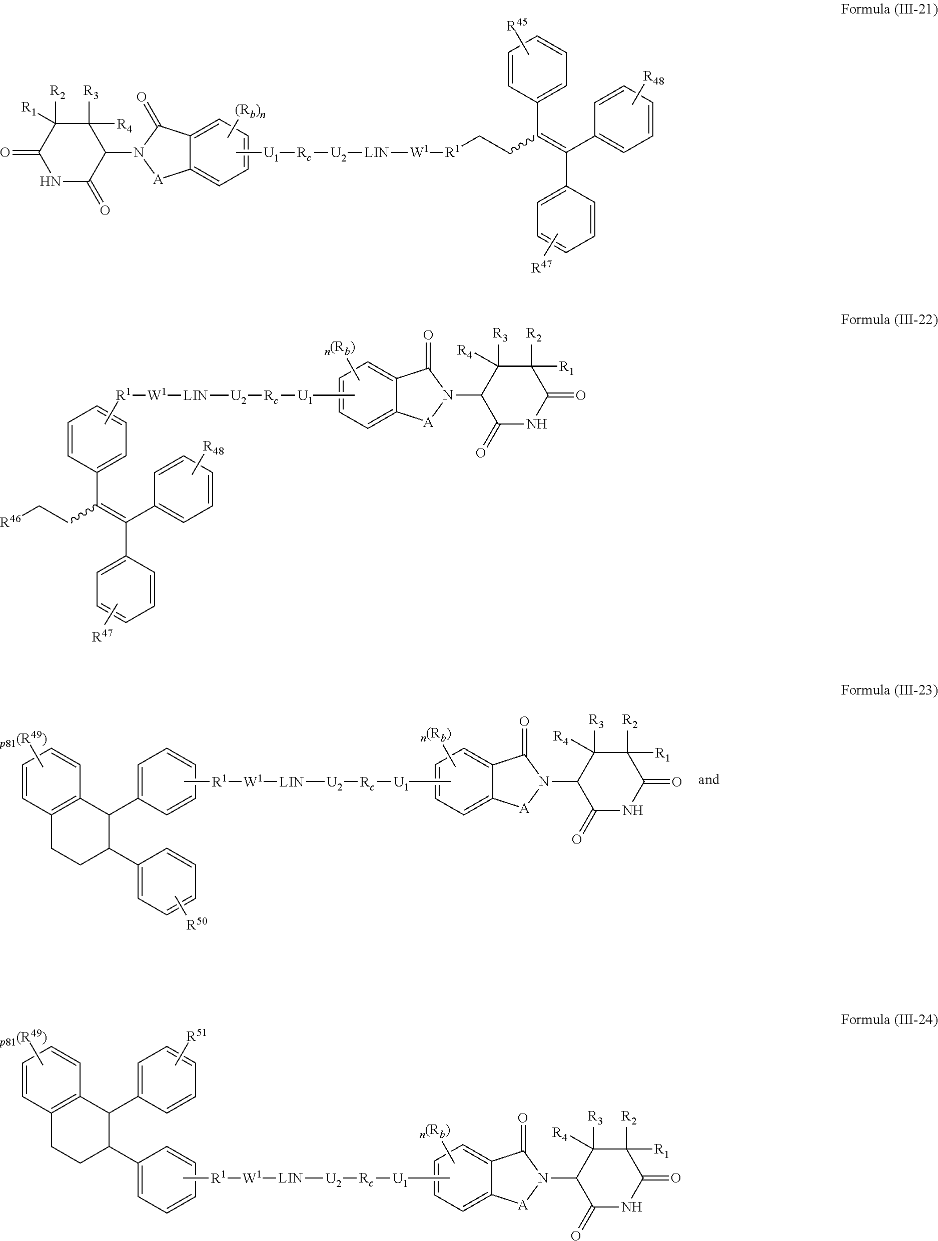

wherein A, R₁, R₂, R₃, R₄, (R_{b})ₙ, U₁, U₂, R_{c}, LIN, W¹, R¹, (R²)ₚ₁, (R³)ₚ₂, (R⁵)ₚ₃, (R⁶)ₚ₄, (R⁷)ₚ₅, R₅, R⁹, R¹⁰, (R¹¹)ₚ₇, ring A, ring B, ring C, X₁, X₂, R¹⁴, R¹⁵, R¹⁶, X₃, X₄, X₅, X₆, R¹⁸, R¹⁹, R²⁰, R²⁵, R²⁶, R²⁷ (R²⁸)ₚ₈, R²⁹, X₇, X₈, X₉, (R³²)ₚ₉, R³³, R³⁴, (R³⁶)ₚ₁₀, (R³⁷)ₚ₁₁, (R³⁸)ₚ₁₂, (R³⁹)ₚ₁₃, (R⁴⁰)ₚ₁₄, R⁴¹, (R⁴²)ₚ₁₅ (R⁴³)ₚ₁₆, (R⁴)ₚ₁₇, R⁴⁵, R⁴⁶, R⁴⁷, R⁴⁸, (R⁴⁹)ₚ₁₈, R⁵⁰, and R⁵¹ are as defined in the compound of Formula (III) above and each sub-embodiments thereof.

In some embodiments, when PBM of the compound of Formula (III) of the present disclosure represents the structures of Formula (PBM-6-1A-1) or Formula (PBM-6-1), and ULM represents the structure of Formula (ULM):

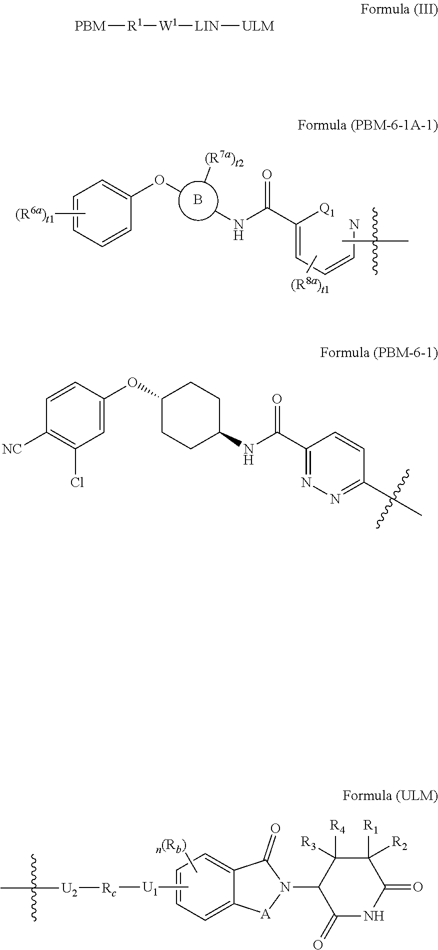

W¹ represents optionally substituted piperidinylene, R_{c} represents optionally substituted piperidinylene, U₁ represents a bond, A, R₁, R₂, R₃, R₄, (R_{b})ₙ, U₂, LIN, R¹, (R^{6a})ₜ₁, ring B, (R^{7a})ₜ₂, (R^{8a})ₜ₃, Q₁ are as defined in the compound of Formula (III) above and each sub-embodiments thereof, wherein substituents of the piperidinylene of W¹ and R_{c} are each independently as defined herein.

In some embodiments, the compounds of Formula (III) of the present invention and their salts (including pharmaceutically acceptable salts, such as hydrochloride, etc.), prodrugs, solvates, isotopically enriched analogs, polymorphs, stereoisomers (including enantiomers and diastereomers), or mixture of stereoisomers thereof in Table 4 below are provided.

TABLE 4
The compounds of the present disclosure

| Compound | | |
|---|---|---|
| No. | Structure of compound | Compound's name |
| GT-03339 | 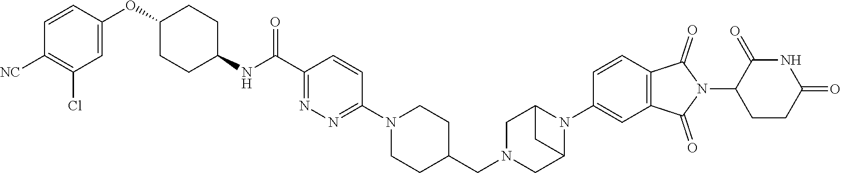 | N-((1r,4r)-4-(3-chloro-4- cyanophenoxy)cyclohexyl)-6-(4-((6-(2-(2,6- dioxopiperidin-3-yl)-1,3-dioxoisoindolin-5-yl)- 3,6-diazabicyclo[3.1.1]heptan-3- yl)methyl)piperidin-1-yl)pyridazine-3- carboxamide |
| GT-03490 | 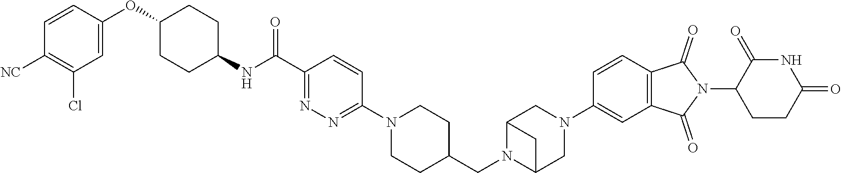 | N-((1r,4r)-4-(3-chloro-4- cyanophenoxy)cyclohexyl)-6-(4-((3-(2-(2,6- dioxopiperidin-3-yl)-1,3-dioxoisoindolin-5-yl)- 3,6-diazabicyclo[3.1.1]heptan-6- yl)methyl)piperidin-1-yl)pyridazine-3- carboxamide |
| GT-03488 | 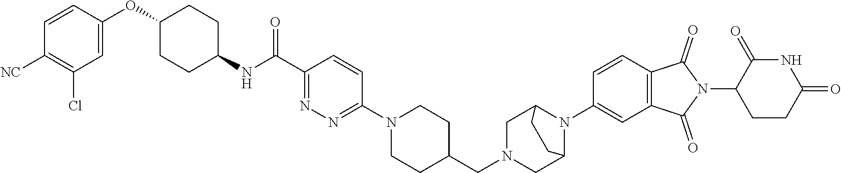 | N-((1r,4r)-4-(3-chloro-4- cyanophenoxy)cyclohexyl)-6-(4-((8-(2-(2,6- dioxopiperidin-3-yl)-1,3-dioxoisoindolin-5-yl)- 3,8-diazabicyclo[3.2.1]octan-3- yl)methyl)piperidin-1-yl)pyridazine-3- carboxamide |
| GT-03489 | 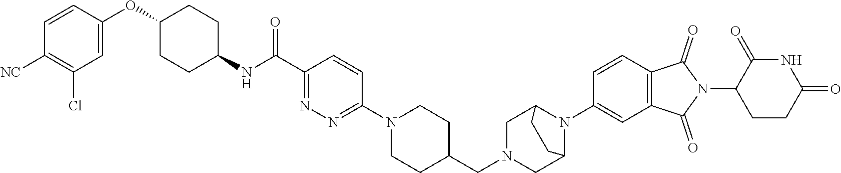 | N-((1r,4r)-4-(3-chloro-4- cyanophenoxy)cyclohexyl)-6-(4-((3-(2-(2,6- dioxopiperidin-3-yl)-1,3-dioxoisoindolin-5-yl)- 3,8-diazabicyclo[3.2.1]octan-8- yl)methyl)piperidin-1-yl)pyridazine-3- carboxamide |
| | 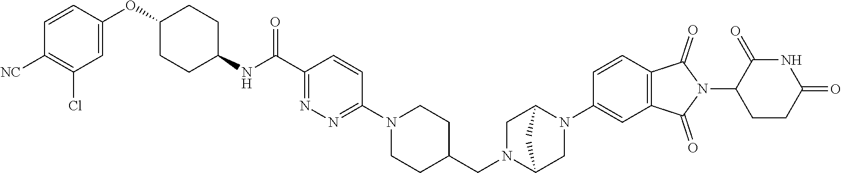 | N-((1r,4r)-4-(3-chloro-4- cyanophenoxy)cyclohexyl)-6-(4-(((1S,4S)-5- (2-(2,6-dioxopiperidin-3-yl)-1,3- dioxoisoindolin-5-yl)-2,5- diazabicyclo[2.2.1]heptan-2- yl)methyl)piperidin-1-yl)pyridazine-3- carboxamide |
| | 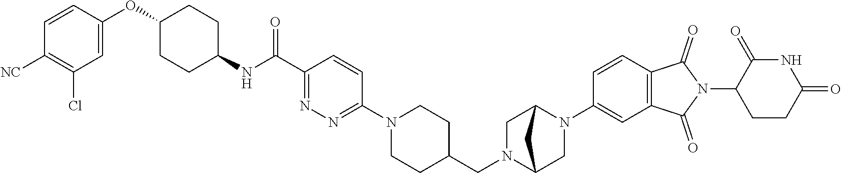 | N-((1r,4r)-4-(3-chloro-4- cyanophenoxy)cyclohexyl)-6-(4-(((1R,4R)-5- (2-(2,6-dioxopiperidin-3-yl)-1,3- dioxoisoindolin-5-yl)-2,5- diazabicyclo[2.2.1]heptan-2- yl)methyl)piperidin-1-yl)pyridazine-3- carboxamide |
| GT-03885 | 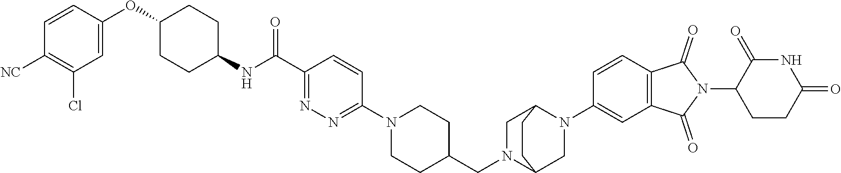 | N-((1r,4r)-4-(3-chloro-4- cyanophenoxy)cyclohexyl)-6-(4-((5-(2-(2,6- dioxopiperidin-3-yl)-1,3-dioxoisoindolin-5-yl)- 2,5-diazabicyclo[2.2.2]octan-2- yl)methyl)piperidin-1-yl)pyridazine-3- carboxamide |
| | 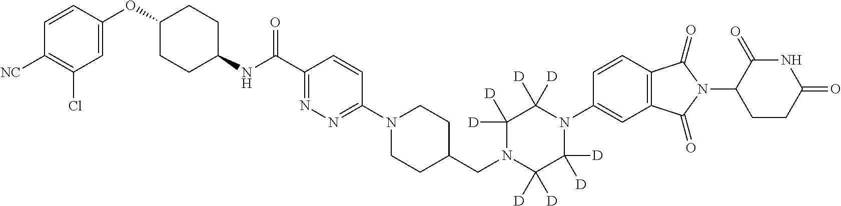 | N-((1r,4r)-4-(3-chloro-4- cyanophenoxy)cyclohexyl)-6-(4-((4-(2-(2,6- dioxopiperidin-3-yl)-1,3-dioxoisoindolin-5- yl)piperazin-1-yl-2,2,3,3,5,5,6,6- d₈)methyl)piperidin-1-yl)pyridazine-3- carboxamide |
| | 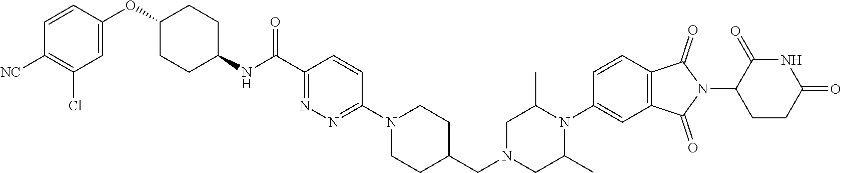 | N-((1r,4r)-4-(3-chloro-4- cyanophenoxy)cyclohexyl)-6-(4-((4-(2-(2,6- dioxopiperidin-3-yl)-1,3-dioxoisoindolin-5-yl)- 3,5-dimethylpiperazin-1-yl)methyl)piperidin-1- yl)pyridazine-3-carboxamide |
| | 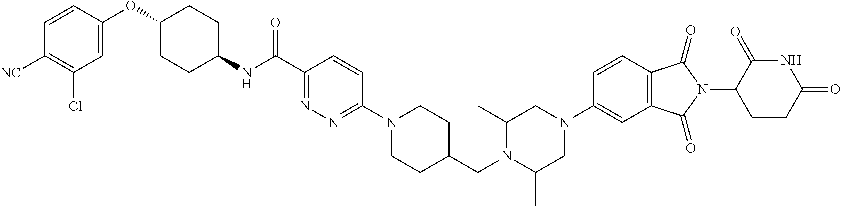 | N-((1r,4r)-4-(3-chloro-4- cyanophenoxy)cyclohexyl)-6-(4-((4-(2-(2,6- dioxopiperidin-3-yl)-1,3-dioxoisoindolin-5-yl)- 2,6-dimethylpiperazin-1-yl)methyl)piperidin-1- yl)pyridazine-3-carboxamide |
| | 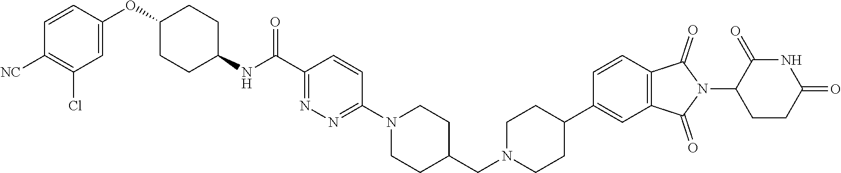 | N-((1r,4r)-4-(3-chloro-4- cyanophenoxy)cyclohexyl)-6-(4-((4-(2-(2,6- dioxopiperidin-3-yl)-1,3-dioxoisoindolin-5- yl)piperidin-1-yl)methyl)piperidin-1- yl)pyridazine-3-carboxamide |
| GT-03486 | 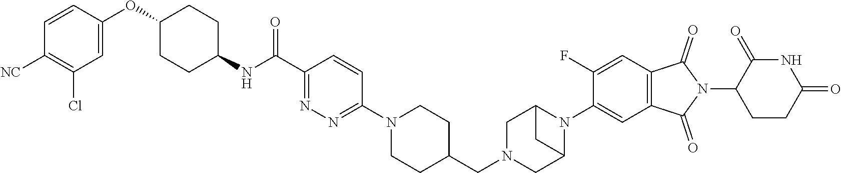 | N-((1r,4r)-4-(3-chloro-4- cyanophenoxy)cyclohexyl)-6-(4-((6-(2-(2,6- dioxopiperidin-3-yl)-6-fluoro-1,3- dioxoisoindolin-5-yl)-3,6- diazabicyclo[3.1.1]heptan-3- yl)methyl)piperidin-1-yl)pyridazine-3- carboxamide |
| GT-03487 | 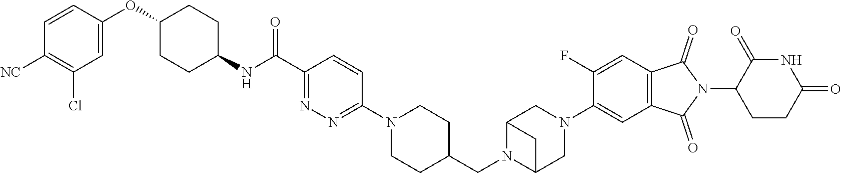 | N-((1r,4r)-4-(3-chloro-4- cyanophenoxy)cyclohexyl)-6-(4-((3-(2-(2,6- dioxopiperidin-3-yl)-6-fluoro-1,3- dioxoisoindolin-5-yl)-3,6- diazabicyclo[3.1.1]heptan-6- yl)methyl)piperidin-1-yl)pyridazine-3- carboxamide |
| GT-02765 | 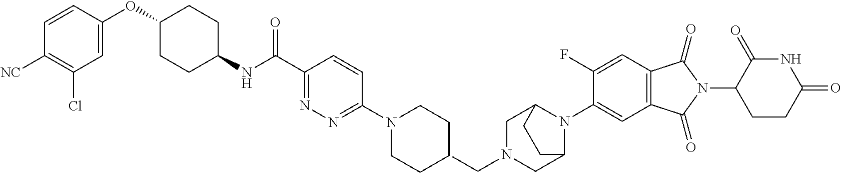 | N-((1r,4r)-4-(3-chloro-4- cyanophenoxy)cyclohexyl)-6-(4-((8-(2-(2,6- dioxopiperidin-3-yl)-6-fluoro-1,3- dioxoisoindolin-5-yl)-3,8- diazabicyclo[3.2.1]octan-3-yl)methyl)piperidin- 1-yl)pyridazine-3-carboxamide |
| GT-02752 | 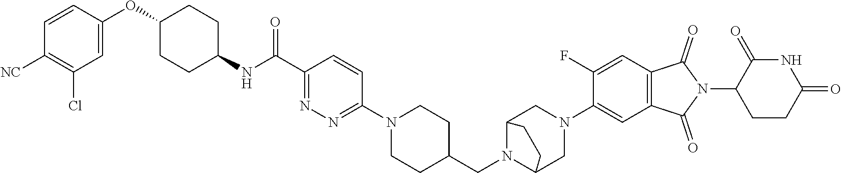 | N-((1r,4r)-4-(3-chloro-4- cyanophenoxy)cyclohexyl)-6-(4-((3-(2-(2,6- dioxopiperidin-3-yl)-6-fluoro-1,3- dioxoisoindolin-5-yl)-3,8- diazabicyclo[3.2.1]octan-8-yl)methyl)piperidin- 1-yl)pyridazine-3-carboxamide |
| GT-03773 | 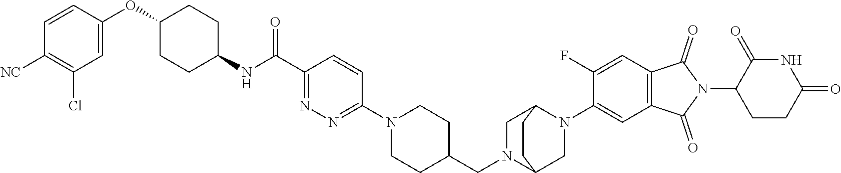 | N-((1r,4r)-4-(3-chloro-4- cyanophenoxy)cyclohexyl)-6-(4-((5-(2-(2,6- dioxopiperidin-3-yl)-6-fluoro-1,3- dioxoisoindolin-5-yl)-2,5- diazabicyclo[2.2.2]octan-2-yl)methyl)piperidin- 1-yl)pyridazine-3-carboxamide |
| | 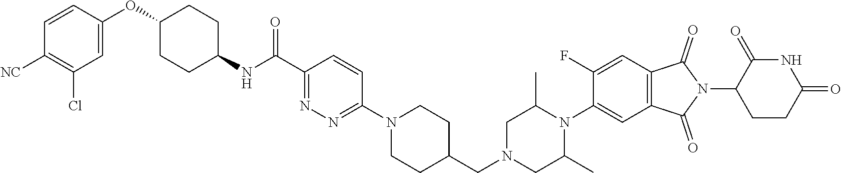 | N-((1r,4r)-4-(3-chloro-4- cyanophenoxy)cyclohexyl)-6-(4-((4-(2-(2,6- dioxopiperidin-3-yl)-6-fluoro-1,3- dioxoisoindolin-5-yl)-3,5-dimethylpiperazin-1- yl)methyl)piperidin-1-yl)pyridazine-3- carboxamide |
| | 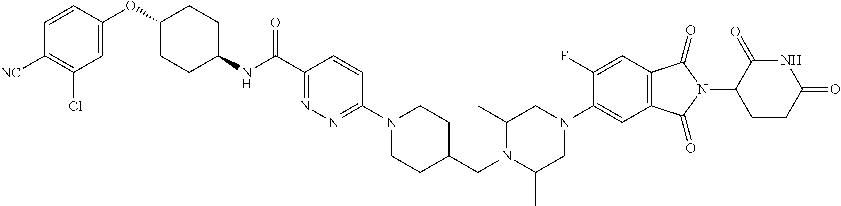 | N-((1r,4r)-4-(3-chloro-4- cyanophenoxy)cyclohexyl)-6-(4-((4-(2-(2,6- dioxopiperidin-3-yl)-6-fluoro-1,3- dioxoisoindolin-5-yl)-2,6-dimethylpiperazin-1- yl)methyl)piperidin-1-yl)pyridazine-3- carboxamide |
| | 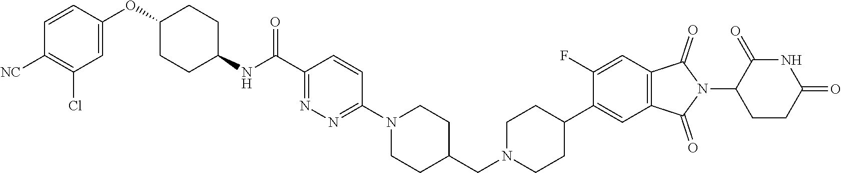 | N-((1r,4r)-4-(3-chloro-4- cyanophenoxy)cyclohexyl)-6-(4-((4-(2-(2,6- dioxopiperidin-3-yl)-6-fluoro-1,3- dioxoisoindolin-5-yl)piperidin-1- yl)methyl)piperidin-1-yl)pyridazine-3- carboxamide |
| GT-03016 | 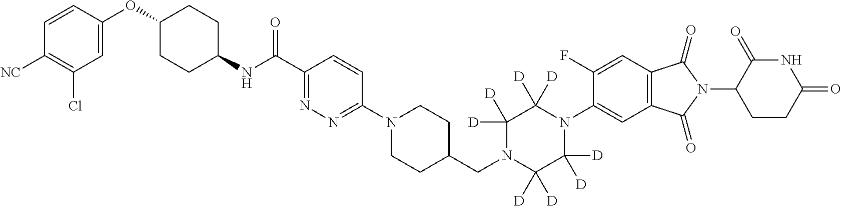 | N-((1r,4r)-4-(3-chloro-4- cyanophenoxy)cyclohexyl)-6-(4-((4-(2-(2,6- dioxopiperidin-3-yl)-6-fluoro-1,3- dioxoisoindolin-5-yl)piperazin-1-yl- 2,2,3,3,5,5,6,6-d₈)methyl)piperidin-1- yl)pyridazine-3-carboxamide |
| | 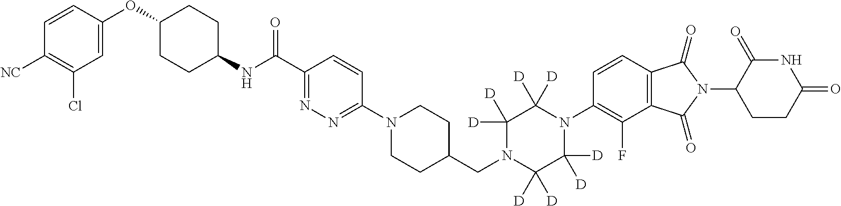 | N-((1r,4r)-4-(3-chloro-4- cyanophenoxy)cyclohexyl)-6-(4-((4-(2-(2,6- dioxopiperidin-3-yl)-4-fluoro-1,3- dioxoisoindolin-5-yl)piperazin-1-yl- 2,2,3,3,5,5,6,6-d₈)methyl)piperidin-1- yl)pyridazine-3-carboxamide |
| | 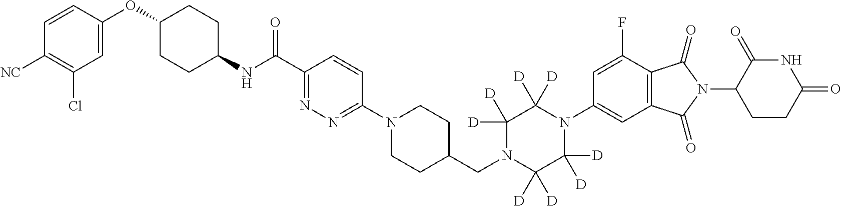 | N-((1r,4r)-4-(3-chloro-4- cyanophenoxy)cyclohexyl)-6-(4-((4-(2-(2,6- dioxopiperidin-3-yl)-7-fluoro-1,3- dioxoisoindolin-5-yl)piperazin-1-yl- 2,2,3,3,5,5,6,6-d₈)methyl)piperidin-1- yl)pyridazine-3-carboxamide |
| | 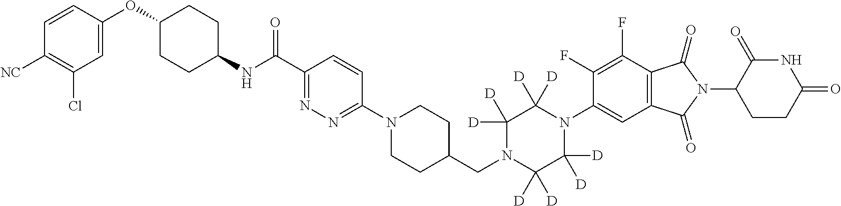 | N-((1r,4r)-4-(3-chloro-4- cyanophenoxy)cyclohexyl)-6-(4-((4-(2-(2,6- dioxopiperidin-3-yl)-6,7-difluoro-1,3- dioxoisoindolin-5-yl)piperazin-1-yl- 2,2,3,3,5,5,6,6-d₈)methyl)piperidin-1- yl)pyridazine-3-carboxamide |
| | 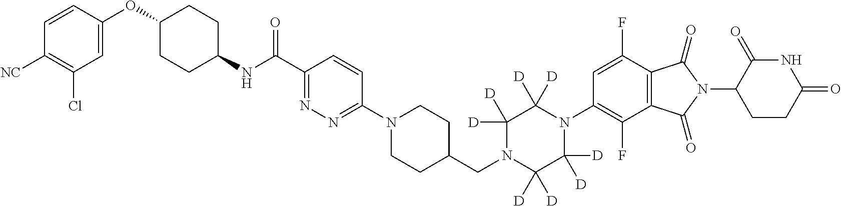 | N-((1r,4r)-4-(3-chloro-4- cyanophenoxy)cyclohexyl)-6-(4-((4-(2-(2,6- dioxopiperidin-3-yl)-4,7-difluoro-1,3- dioxoisoindolin-5-yl)piperazin-1-yl- 2,2,3,3,5,5,6,6-d₈)methyl)piperidin-1- yl)pyridazine-3-carboxamide |
| | 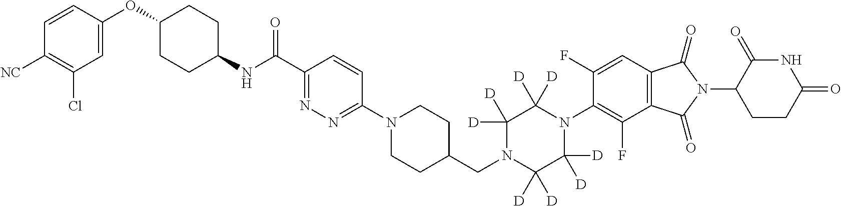 | N-((1r,4r)-4-(3-chloro-4- cyanophenoxy)cyclohexyl)-6-(4-((4-(2-(2,6- dioxopiperidin-3-yl)-4,6-difluoro-1,3- dioxoisoindolin-5-yl)piperazin-1-yl- 2,2,3,3,5,5,6,6-d₈)methyl)piperidin-1- yl)pyridazine-3-carboxamide |
| | 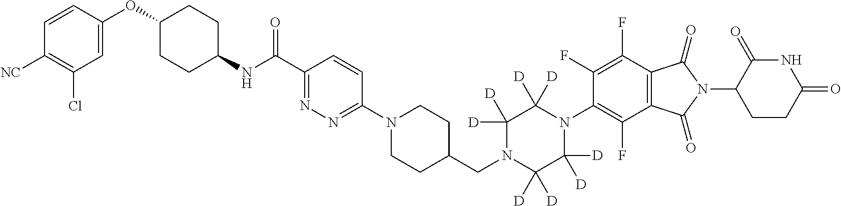 | N-((1r,4r)-4-(3-chloro-4- cyanophenoxy)cyclohexyl)-6-(4-((4-(2-(2,6- dioxopiperidin-3-yl)-4,6,7-trifluoro-1,3- dioxoisoindolin-5-yl)piperazin-1-yl- 2,2,3,3,5,5,6,6-d₈)methyl)piperidin-1- yl)pyridazine-3-carboxamide |
| | 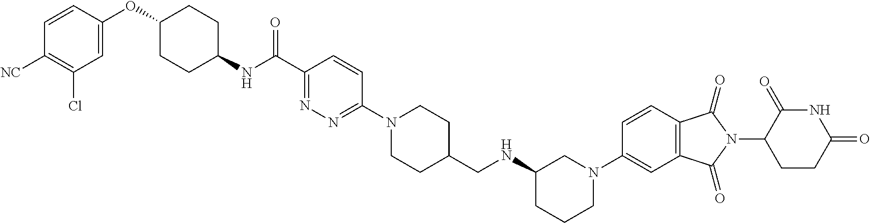 | N-((1r,4r)-4-(3-chloro-4- cyanophenoxy)cyclohexyl)-6-(4-((((3R)-1-(2- (2,6-dioxopiperidin-3-yl)-1,3-dioxoisoindolin- 5-yl)piperidin-3-yl)amino)methyl)piperidin-1- yl)pyridazine-3-carboxamide |
| | 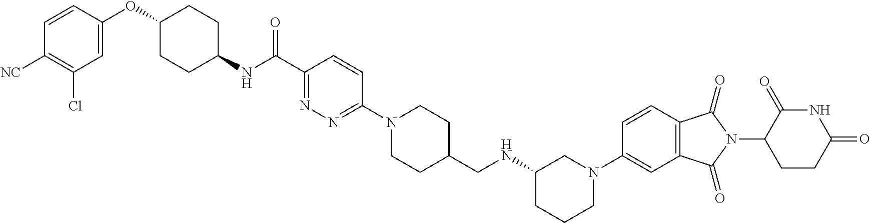 | N-((1r,4r)-4-(3-chloro-4- cyanophenoxy)cyclohexyl)-6-(4-((((3S)-1-(2- (2,6-dioxopiperidin-3-yl)-1,3-dioxoisoindolin- 5-yl)piperidin-3-yl)amino)methyl)piperidin-1- yl)pyridazine-3-carboxamide |
| | 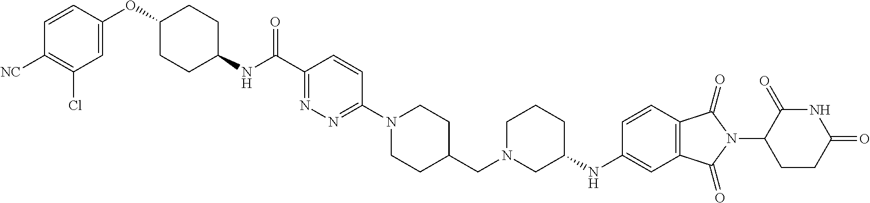 | N-((1r,4r)-4-(3-chloro-4- cyanophenoxy)cyclohexyl)-6-(4-(((3S)-3-((2- (2,6-dioxopiperidin-3-yl)-1,3-dioxoisoindolin- 5-yl)amino)piperidin-1-yl)methyl)piperidin-1- yl)pyridazine-3-carboxamide |
| | 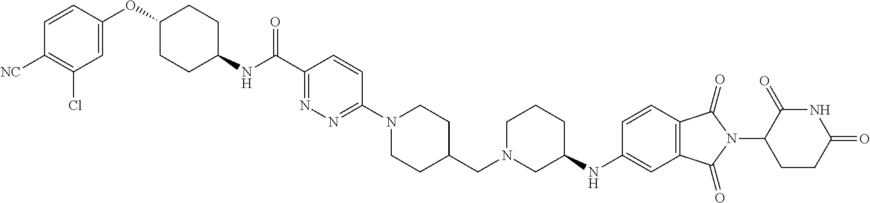 | N-((1r,4r)-4-(3-chloro-4- cyanophenoxy)cyclohexyl)-6-(4-(((3R)-3-((2- (2,6-dioxopiperidin-3-yl)-1,3-dioxoisoindolin- 5-yl)amino)piperidin-1-yl)methyl)piperidin-1- yl)pyridazine-3-carboxamide |
| GT-03449 | 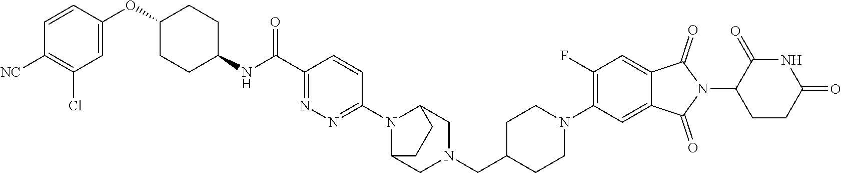 | N-((1r,4r)-4-(3-chloro-4- cyanophenoxy)cyclohexyl)-6-(3-((1-(2-(2,6- dioxopiperidin-3-yl)-6-fluoro-1,3- dioxoisoindolin-5-yl)piperidin-4-yl)methyl)- 3,8-diazabicyclo[3.2.1]octan-8-yl)pyridazine-3- carboxamide |
| GT-03450 | 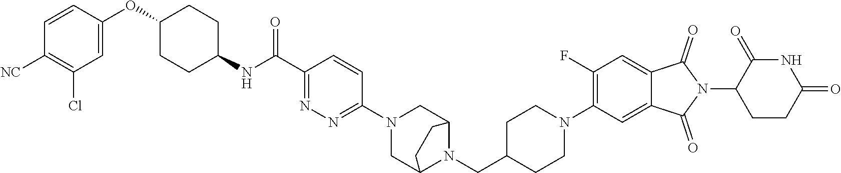 | N-((1r,4r)-4-(3-chloro-4- cyanophenoxy)cyclohexyl)-6-(8-((1-(2-(2,6- dioxopiperidin-3-yl)-6-fluoro-1,3- dioxoisoindolin-5-yl)piperidin-4-yl)methyl)- 3,8-diazabicyclo[3.2.1]octan-3-yl)pyridazine-3- carboxamide |
| GT-03451 | 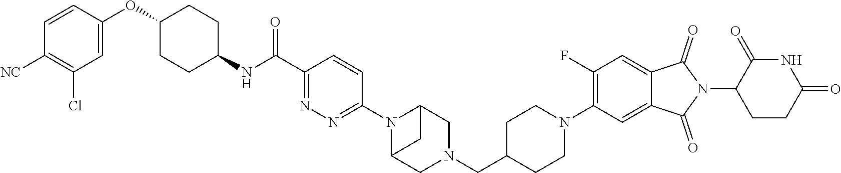 | N-((1r,4r)-4-(3-chloro-4- cyanophenoxy)cyclohexyl)-6-(3-((1-(2-(2,6- dioxopiperidin-3-yl)-6-fluoro-1,3- dioxoisoindolin-5-yl)piperidin-4-yl)methyl)- 3,6-diazabicyclo[3.1.1]heptan-6-yl)pyridazine- 3-carboxamide |
| GT-03470 | 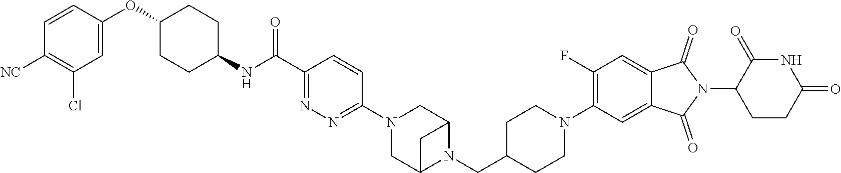 | N-((1r,4r)-4-(3-chloro-4- cyanophenoxy)cyclohexyl)-6-(6-((1-(2-(2,6- dioxopiperidin-3-yl)-6-fluoro-1,3- dioxoisoindolin-5-yl)piperidin-4-yl)methyl)- 3,6-diazabicyclo[3.1.1]heptan-3-yl)pyridazine- 3-carboxamide |
| GT-03427 | 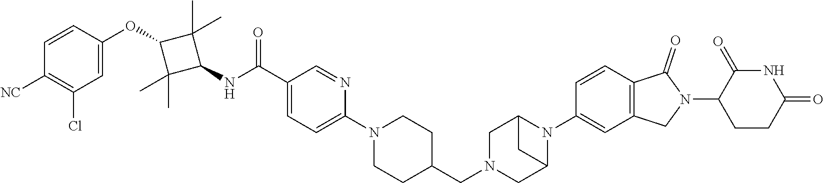 [SMILES: CC1(C)[C@H](NC(=O)c2ccc(N3CCC(CN4CC5CC(C4)N5c4ccc5c(c4)CN(C4CCC(=O)NC4=O)C5=O)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((6-(2-(2,6- dioxopiperidin-3-yl)-1,3-dioxoisoindolin-5-yl)- 3,6-diazabicyclo[3.1.1]heptan-3- yl)methyl)piperidin-1-yl)nicotinamide |
| GT-03471 | 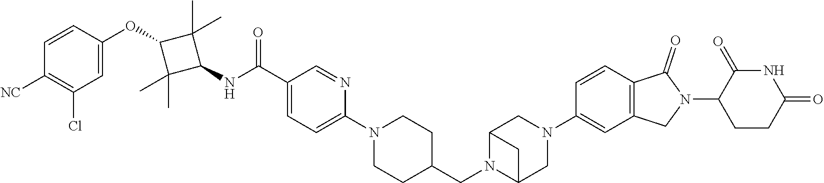 [SMILES: CC1(C)[C@H](NC(=O)c2ccc(N3CCC(CN4C5CC4CN(c4ccc6c(c4)CN(C4CCC(=O)NC4=O)C6=O)C5)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((3-(2-(2,6- dioxopiperidin-3-yl)-1,3-dioxoisoindolin-5-yl)- 3,6-diazabicyclo[3.1.1]heptan-6- yl)methyl)piperidin-1-yl)nicotinamide |
| GT-03425 | 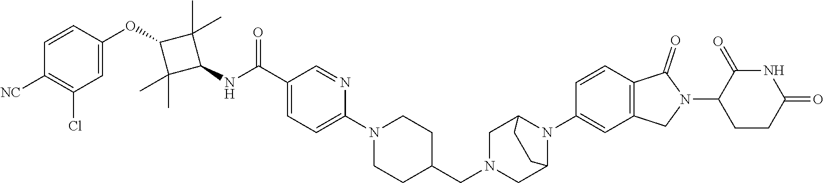 [SMILES: CC1(C)[C@H](NC(=O)c2ccc(N3CCC(CN4CC5CCC(C4)N5c4ccc5c(c4)CN(C4CCC(=O)NC4=O)C5=O)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((8-(2-(2,6- dioxopiperidin-3-yl)-1,3-dioxoisoindolin-5-yl)- 3,8-diazabicyclo[3.2.1]octan-3- yl)methyl)piperidin-1-yl)nicotinamide |
| GT-03426 | 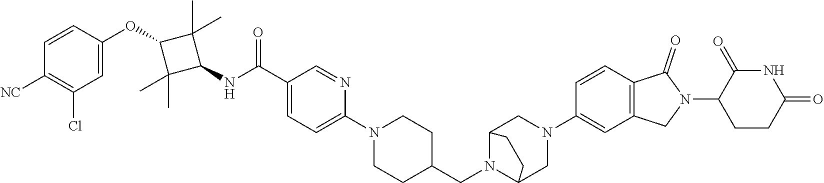 [SMILES: CC1(C)[C@H](NC(=O)c2ccc(N3CCC(CN4C5CCC4CN(c4ccc6c(c4)CN(C4CCC(=O)NC4=O)C6=O)C5)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((3-(2-(2,6- dioxopiperidin-3-yl)-1,3-dioxoisoindolin-5-yl)- 3,8-diazabicyclo[3.2.1]octan-8- yl)methyl)piperidin-1-yl)nicotinamide |
| | 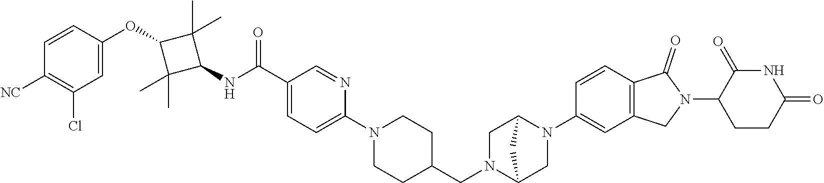 [SMILES: CC1(C)[C@H](NC(=O)c2ccc(N3CCC(CN4C[C@@H]5C[C@H]4CN5c4ccc5c(c4)CN(C4CCC(=O)NC4=O)C5=O)CC3)nc2)C(C)(C)[C@H]1Oc1ccc(C#N)c(Cl)c1] | N-((1r,3S)-3-(3-chloro-4-cyanophenoxy)- 2,2,4,4-tetramethylcyclobutyl)-6-(4-(((1S,4S)- 5-(2-(2,6-dioxopiperidin-3-yl)-1,3- dioxoisoindolin-5-yl)-2,5- diazabicyclo[2.2.1]heptan-2- yl)methyl)piperidin-1-yl)nicotinamide |
| | 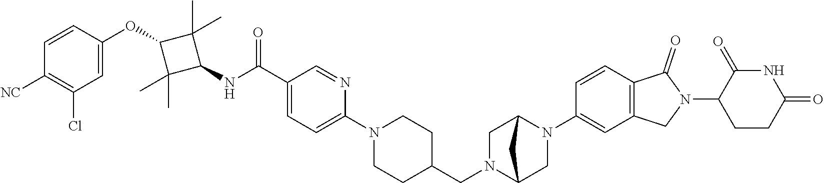 [SMILES: CC1(C)[C@H](NC(=O)c2ccc(N3CCC(CN4C[C@H]5C[C@@H]4CN5c4ccc5c(c4)CN(C4CCC(=O)NC4=O)C5=O)CC3)nc2)C(C)(C)[C@H]1Oc1ccc(C#N)c(Cl)c1] | N-((1r,3R)-3-(3-chloro-4-cyanophenoxy)- 2,2,4,4-tetramethylcyclobutyl)-6-(4-(((1R,4R)- 5-(2-(2,6-dioxopiperidin-3-yl)-1,3- dioxoisoindolin-5-yl)-2,5- diazabicyclo[2.2.1]heptan-2- yl)methyl)piperidin-1-yl)nicotinamide |
| GT-03481 | 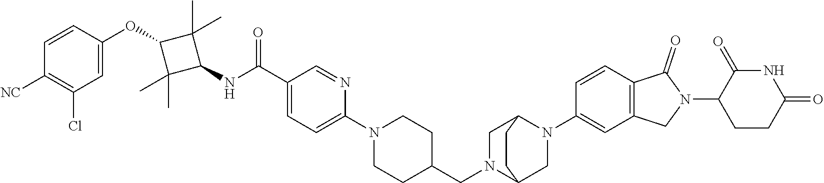 [SMILES: CC1(C)[C@H](NC(=O)c2ccc(N3CCC(CN4CC5CCC4CN5c4ccc5c(c4)CN(C4CCC(=O)NC4=O)C5=O)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((5-(2-(2,6- dioxopiperidin-3-yl)-1,3-dioxoisoindolin-5-yl)- 2,5-diazabicyclo[2.2.2]octan-2- yl)methyl)piperidin-1-yl)nicotinamide |
| | 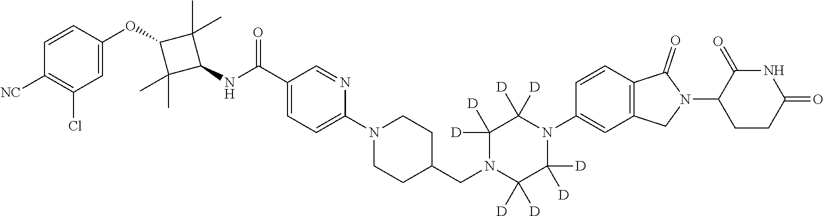 [SMILES: [2H]C1([2H])N(CC2CCN(c3ccc(C(=O)N[C@H]4C(C)(C)[C@H](Oc5ccc(C#N)c(Cl)c5)C4(C)C)cn3)CC2)C([2H])([2H])C([2H])([2H])N(c2ccc3c(c2)CN(C2CCC(=O)NC2=O)C3=O)C1([2H])[2H]] | N-((1r,3r)-3-(3-chloro-4-cyanophenoxy)- 2,2,4,4-tetramethylcyclobutyl)-6-(4-((4-(2-(2,6- dioxopiperidin-3-yl)-1,3-dioxoisoindolin-5- yl)piperazin-1-yl-2,2,3,3,5,5,6,6- d₈)methyl)piperidin-1-yl)nicotinamide |
| | 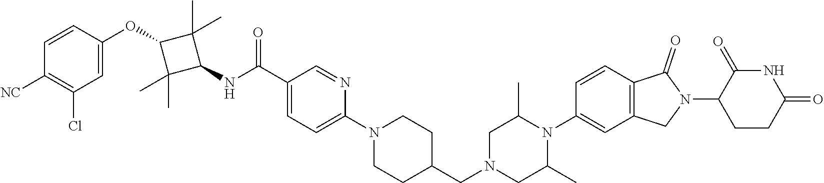 [SMILES: CC1CN(CC2CCN(c3ccc(C(=O)N[C@H]4C(C)(C)[C@H](Oc5ccc(C#N)c(Cl)c5)C4(C)C)cn3)CC2)CC(C)N1c1ccc2c(c1)CN(C1CCC(=O)NC1=O)C2=O] | N-((1r,3r)-3-(3-chloro-4-cyanophenoxy)- 2,2,4,4-tetramethylcyclobutyl)-6-(4-((4-(2-(2,6- dioxopiperidin-3-yl)-1,3-dioxoisoindolin-5-yl)- 3,5-dimethylpiperazin-1-yl)methyl)piperidin-1- yl)nicotinamide |
| | 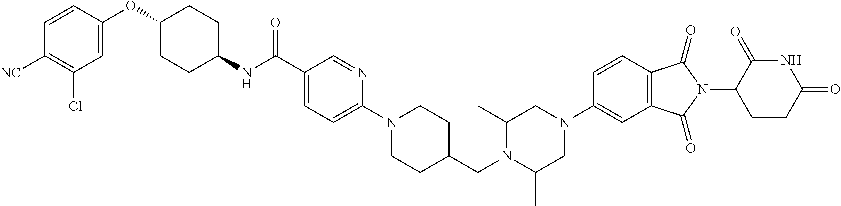 [SMILES: CC1CN(c2ccc3c(c2)C(=O)N(C2CCC(=O)NC2=O)C3=O)CC(C)N1CC1CCN(c2ccc(C(=O)N[C@H]3CC[C@H](Oc4ccc(C#N)c(Cl)c4)CC3)cn2)CC1] | N-((1r,3r)-3-(3-chloro-4-cyanophenoxy)- 2,2,4,4-tetramethylcyclobutyl)-6-(4-((4-(2-(2,6- dioxopiperidin-3-yl)-1,3-dioxoisoindolin-5-yl)- 2,6-dimethylpiperazin-1-yl)methyl)piperidin-1- yl)nicotinamide |
| | 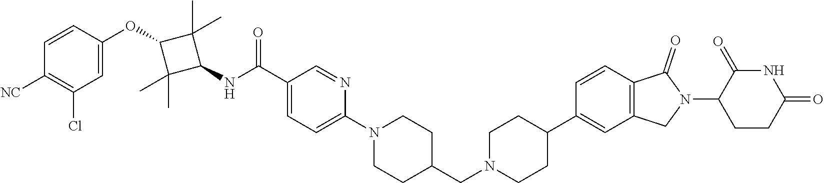 [SMILES: CC1(C)[C@H](NC(=O)c2ccc(N3CCC(CN4CCC(c5ccc6c(c5)CN(C5CCC(=O)NC5=O)C6=O)CC4)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((4-(2-(2,6- dioxopiperidin-3-yl)-1,3-dioxoisoindolin-5- yl)piperidin-1-yl)methyl)piperidin-1- yl)nicotinamide |
| GT-03403 | 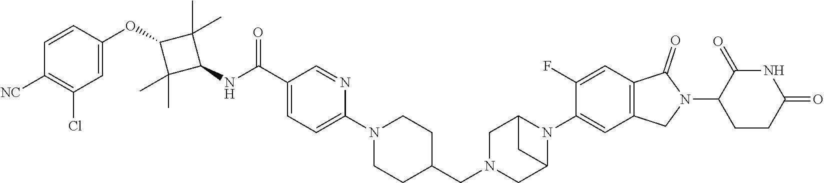 [SMILES: CC1(C)[C@H](NC(=O)c2ccc(N3CCC(CN4CC5CC(C4)N5c4cc5c(cc4F)C(=O)N(C4CCC(=O)NC4=O)C5)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((6-(2-(2,6- dioxopiperidin-3-yl)-6-fluoro-1,3- dioxoisoindolin-5-yl)-3,6- diazabicyclo[3.1.1]heptan-3- yl)methyl)piperidin-1-yl)nicotinamide |
| GT-03404 | 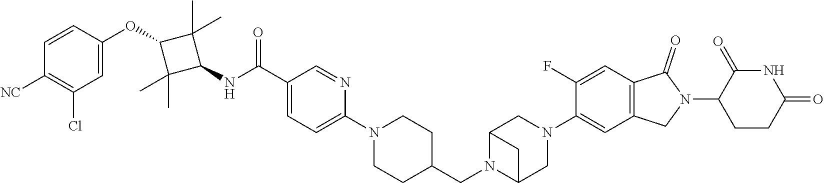 [SMILES: CC1(C)[C@H](NC(=O)c2ccc(N3CCC(CN4C5CC4CN(c4cc6c(cc4F)C(=O)N(C4CCC(=O)NC4=O)C6)C5)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((3-(2-(2,6- dioxopiperidin-3-yl)-6-fluoro-1,3- dioxoisoindolin-5-yl)-3,6- diazabicyclo[3.1.1]heptan-6- yl)methyl)piperidin-1-yl)nicotinamide |
| GT-03402 | 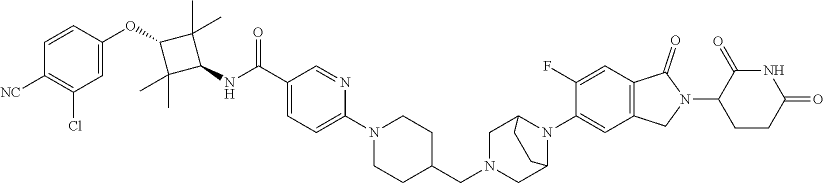 [SMILES: CC1(C)[C@H](NC(=O)c2ccc(N3CCC(CN4CC5CCC(C4)N5c4cc5c(cc4F)C(=O)N(C4CCC(=O)NC4=O)C5)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((8-(2-(2,6- dioxopiperidin-3-yl)-6-fluoro-1,3- dioxoisoindolin-5-yl)-3,8- diazabicyclo[3.2.1]octan-3-yl)methyl)piperidin- 1-yl)nicotinamide |
| GT-03482 | 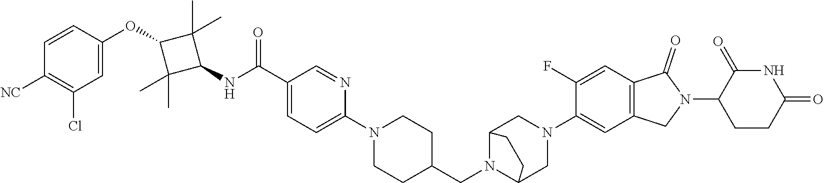 [SMILES: CC1(C)[C@H](NC(=O)c2ccc(N3CCC(CN4C5CCC4CN(c4cc6c(cc4F)C(=O)N(C4CCC(=O)NC4=O)C6)C5)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((3-(2-(2,6- dioxopiperidin-3-yl)-6-fluoro-1,3- dioxoisoindolin-5-yl)-3,8- diazabicyclo[3.2.1]octan-8-yl)methyl)piperidin- 1-yl)nicotinamide |
| GT-03494 | 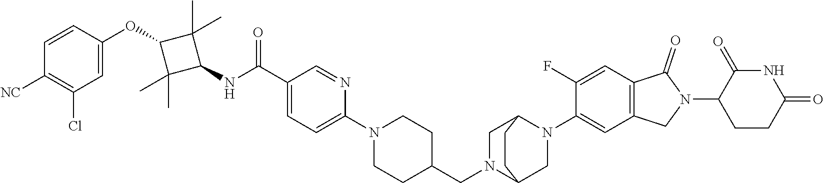 [SMILES: CC1(C)[C@H](NC(=O)c2ccc(N3CCC(CN4CC5CCC4CN5c4cc5c(cc4F)C(=O)N(C4CCC(=O)NC4=O)C5)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((5-(2-(2,6- dioxopiperidin-3-yl)-6-fluoro-1,3- dioxoisoindolin-5-yl)-2,5- diazabicyclo[2.2.2]octan-2-yl)methyl)piperidin- 1-yl)nicotinamide |
| | 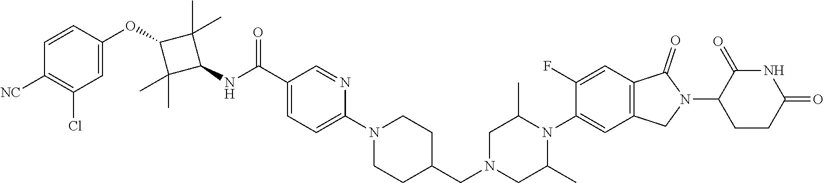 [SMILES: CC1CN(CC2CCN(c3ccc(C(=O)N[C@H]4C(C)(C)[C@H](Oc5ccc(C#N)c(Cl)c5)C4(C)C)cn3)CC2)CC(C)N1c1cc2c(cc1F)C(=O)N(C1CCC(=O)NC1=O)C2] | N-((1r,3r)-3-(3-chloro-4-cyanophenoxy)- 2,2,4,4-tetramethylcyclobutyl)-6-(4-((4-(2-(2,6- dioxopiperidin-3-yl)-6-fluoro-1,3- dioxoisoindolin-5-yl)-3,5-dimethylpiperazin-1- yl)methyl)piperidin-1-yl)nicotinamide |
| | 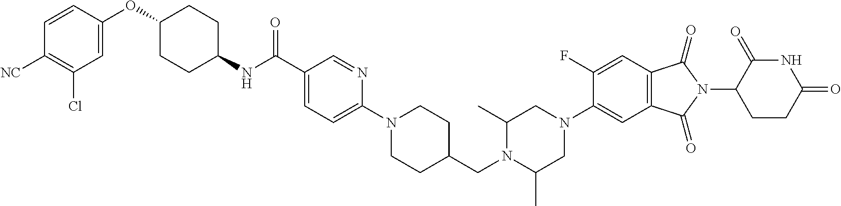 [SMILES: CC1CN(c2cc3c(cc2F)C(=O)N(C2CCC(=O)NC2=O)C3=O)CC(C)N1CC1CCN(c2ccc(C(=O)N[C@H]3CC[C@H](Oc4ccc(C#N)c(Cl)c4)CC3)cn2)CC1] | N-((1r,3r)-3-(3-chloro-4-cyanophenoxy)- 2,2,4,4-tetramethylcyclobutyl)-6-(4-((4-(2-(2,6- dioxopiperidin-3-yl)-6-fluoro-1,3- dioxoisoindolin-5-yl)-2,6-dimethylpiperazin-1- yl)methyl)piperidin-1-yl)nicotinamide |
| | 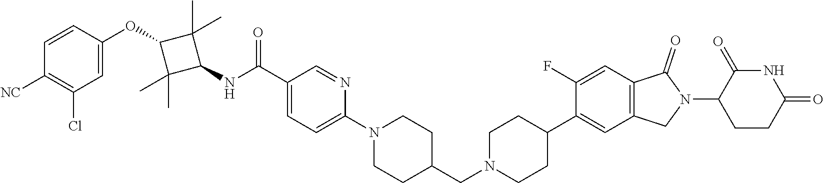 [SMILES: CC1(C)[C@H](NC(=O)c2ccc(N3CCC(CN4CCC(c5cc6c(cc5F)C(=O)N(C5CCC(=O)NC5=O)C6)CC4)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((4-(2-(2,6- dioxopiperidin-3-yl)-6-fluoro-1,3- dioxoisoindolin-5-yl)piperidin-1- yl)methyl)piperidin-1-yl)nicotinamide |
| | 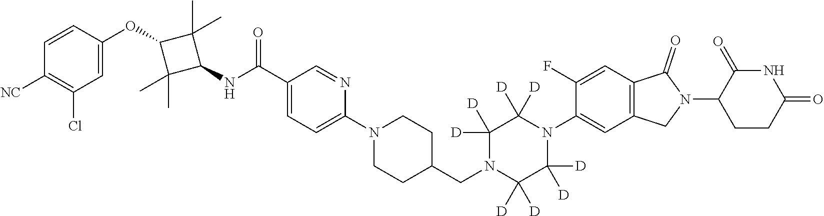 [SMILES: [2H]C1([2H])N(CC2CCN(c3ccc(C(=O)N[C@H]4C(C)(C)[C@H](Oc5ccc(C#N)c(Cl)c5)C4(C)C)cn3)CC2)C([2H])([2H])C([2H])([2H])N(c2cc3c(cc2F)C(=O)N(C2CCC(=O)NC2=O)C3)C1([2H])[2H]] | N-((1r,3r)-3-(3-chloro-4-cyanophenoxy)- 2,2,4,4-tetramethylcyclobutyl)-6-(4-((4-(2-(2,6- dioxopiperidin-3-yl)-6-fluoro-1,3- dioxoisoindolin-5-yl)piperazin-1-yl- 2,2,3,3,5,5,6,6-d₈)methyl)piperidin-1- yl)nicotinamide |
| | 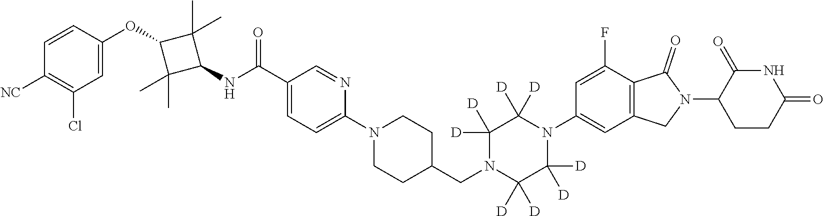 [SMILES: [2H]C1([2H])N(CC2CCN(c3ccc(C(=O)N[C@H]4C(C)(C)[C@H](Oc5ccc(C#N)c(Cl)c5)C4(C)C)cn3)CC2)C([2H])([2H])C([2H])([2H])N(c2cc(F)c3c(c2)CN(C2CCC(=O)NC2=O)C3=O)C1([2H])[2H]] | N-((1r,3r)-3-(3-chloro-4-cyanophenoxy)- 2,2,4,4-tetramethylcyclobutyl)-6-(4-((4-(2-(2,6- dioxopiperidin-3-yl)-7-fluoro-1,3- dioxoisoindolin-5-yl)piperazin-1-yl- 2,2,3,3,5,5,6,6-d₈)methyl)piperidin-1- yl)nicotinamide |
| | 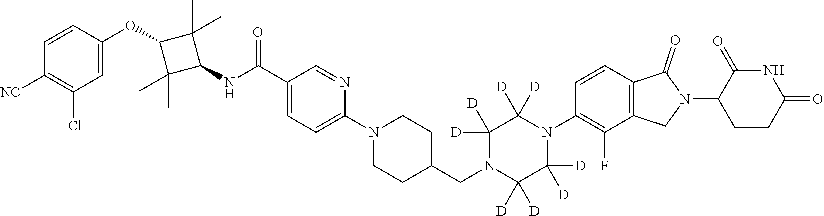 [SMILES: [2H]C1([2H])N(CC2CCN(c3ccc(C(=O)N[C@H]4C(C)(C)[C@H](Oc5ccc(C#N)c(Cl)c5)C4(C)C)cn3)CC2)C([2H])([2H])C([2H])([2H])N(c2ccc3c(c2F)CN(C2CCC(=O)NC2=O)C3=O)C1([2H])[2H]] | N-((1r,3r)-3-(3-chloro-4-cyanophenoxy)- 2,2,4,4-tetramethylcyclobutyl)-6-(4-((4-(2-(2,6- dioxopiperidin-3-yl)-4-fluoro-1,3- dioxoisoindolin-5-yl)piperazin-1-yl- 2,2,3,3,5,5,6,6-d₈)methyl)piperidin-1- yl)nicotinamide |
| | 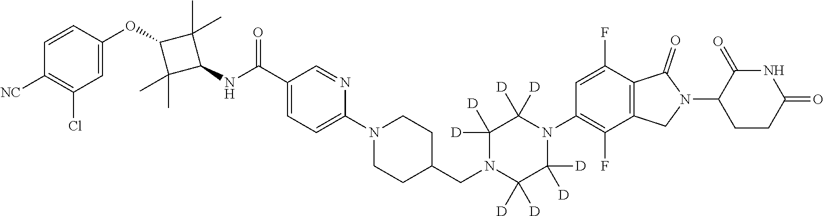 [SMILES: [2H]C1([2H])N(CC2CCN(c3ccc(C(=O)N[C@H]4C(C)(C)[C@H](Oc5ccc(C#N)c(Cl)c5)C4(C)C)cn3)CC2)C([2H])([2H])C([2H])([2H])N(c2cc(F)c3c(c2F)CN(C2CCC(=O)NC2=O)C3=O)C1([2H])[2H]] | N-((1r,3r)-3-(3-chloro-4-cyanophenoxy)- 2,2,4,4-tetramethylcyclobutyl)-6-(4-((4-(2-(2,6- dioxopiperidin-3-yl)-4,7-difluoro-1,3- dioxoisoindolin-5-yl)piperazin-1-yl- 2,2,3,3,5,5,6,6-d₈)methyl)piperidin-1- yl)nicotinamide |
| | 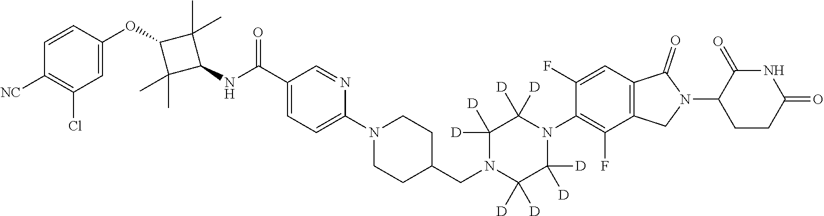 [SMILES: [2H]C1([2H])N(CC2CCN(c3ccc(C(=O)N[C@H]4C(C)(C)[C@H](Oc5ccc(C#N)c(Cl)c5)C4(C)C)cn3)CC2)C([2H])([2H])C([2H])([2H])N(c2c(F)cc3c(c2F)CN(C2CCC(=O)NC2=O)C3=O)C1([2H])[2H]] | N-((1r,3r)-3-(3-chloro-4-cyanophenoxy)- 2,2,4,4-tetramethylcyclobutyl)-6-(4-((4-(2-(2,6- dioxopiperidin-3-yl)-4,6-difluoro-1,3- dioxoisoindolin-5-yl)piperazin-1-yl- 2,2,3,3,5,5,6,6-d₈)methyl)piperidin-1- yl)nicotinamide |
| | 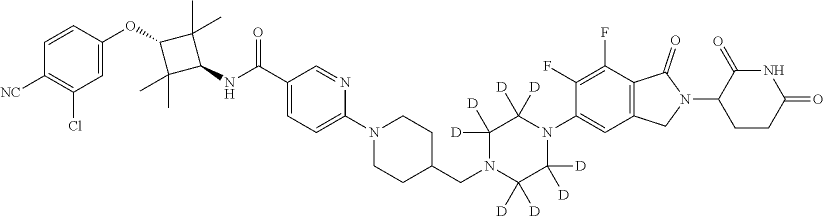 [SMILES: [2H]C1([2H])N(CC2CCN(c3ccc(C(=O)N[C@H]4C(C)(C)[C@H](Oc5ccc(C#N)c(Cl)c5)C4(C)C)cn3)CC2)C([2H])([2H])C([2H])([2H])N(c2cc3c(c(F)c2F)C(=O)N(C2CCC(=O)NC2=O)C3)C1([2H])[2H]] | N-((1r,3r)-3-(3-chloro-4-cyanophenoxy)- 2,2,4,4-tetramethylcyclobutyl)-6-(4-((4-(2-(2,6- dioxopiperidin-3-yl)-6,7-difluoro-1,3- dioxoisoindolin-5-yl)piperazin-1-yl- 2,2,3,3,5,5,6,6-d₈)methyl)piperidin-1- yl)nicotinamide |
| | 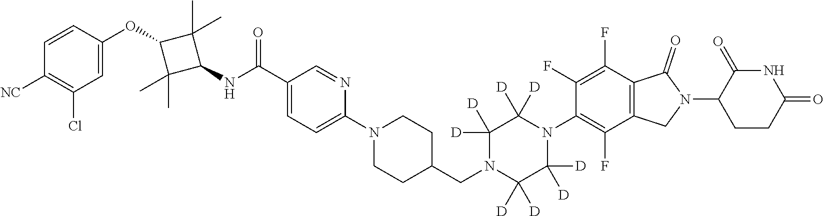 [SMILES: [2H]C1([2H])N(CC2CCN(c3ccc(C(=O)N[C@H]4C(C)(C)[C@H](Oc5ccc(C#N)c(Cl)c5)C4(C)C)cn3)CC2)C([2H])([2H])C([2H])([2H])N(c2c(F)c(F)c3c(c2F)CN(C2CCC(=O)NC2=O)C3=O)C1([2H])[2H]] | N-((1r,3r)-3-(3-chloro-4-cyanophenoxy)- 2,2,4,4-tetramethylcyclobutyl)-6-(4-((4-(2-(2,6- dioxopiperidin-3-yl)-4,6,7-trifluoro-1,3- dioxoisoindolin-5-yl)piperazin-1-yl- 2,2,3,3,5,5,6,6-d₈)methyl)piperidin-1- yl)nicotinamide |
| | 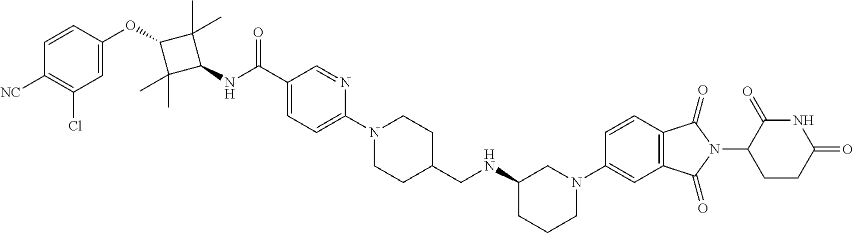 [SMILES: CC1(C)[C@H](NC(=O)c2ccc(N3CCC(CN[C@@H]4CCCN(c5ccc6c(c5)C(=O)N(C5CCC(=O)NC5=O)C6=O)C4)CC3)nc2)C(C)(C)[C@H]1Oc1ccc(C#N)c(Cl)c1] | N-((1r,3R)-3-(3-chloro-4-cyanophenoxy)- 2,2,4,4-tetramethylcyclobutyl)-6-(4-((((3R)-1- (2-(2,6-dioxopiperidin-3-yl)-1,3- dioxoisoindolin-5-yl)piperidin-3- yl)amino)methyl)piperidin-1-yl)nicotinamide |
| | 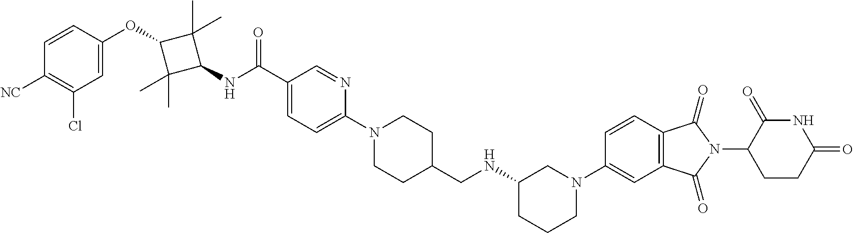 [SMILES: CC1(C)[C@H](NC(=O)c2ccc(N3CCC(CN[C@H]4CCCN(c5ccc6c(c5)C(=O)N(C5CCC(=O)NC5=O)C6=O)C4)CC3)nc2)C(C)(C)[C@H]1Oc1ccc(C#N)c(Cl)c1] | N-((1r,3S)-3-(3-chloro-4-cyanophenoxy)- 2,2,4,4-tetramethylcyclobutyl)-6-(4-((((3S)-1- (2-(2,6-dioxopiperidin-3-yl)-1,3- dioxoisoindolin-5-yl)piperidin-3- yl)amino)methyl)piperidin-1-yl)nicotinamide |
| | 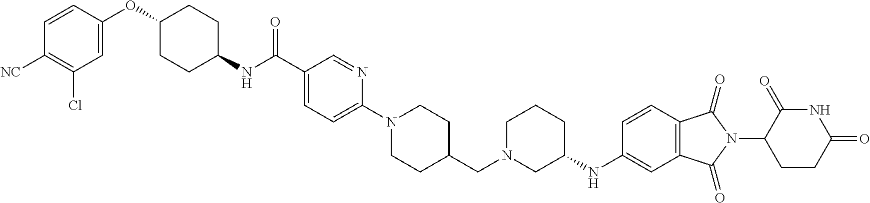 [SMILES: N#Cc1ccc(O[C@H]2CC[C@H](NC(=O)c3ccc(N4CCC(CN5CCC[C@H](Nc6ccc7c(c6)C(=O)N(C6CCC(=O)NC6=O)C7=O)C5)CC4)nc3)CC2)cc1Cl] | N-((1r,3r)-3-(3-chloro-4-cyanophenoxy)- 2,2,4,4-tetramethylcyclobutyl)-6-(4-(((3S)-3- ((2-(2,6-dioxopiperidin-3-yl)-1,3- dioxoisoindolin-5-yl)amino)piperidin-1- yl)methyl)piperidin-1-yl)nicotinamide |
| | 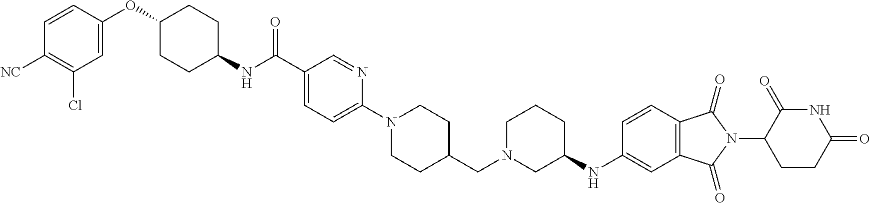 [SMILES: N#Cc1ccc(O[C@H]2CC[C@H](NC(=O)c3ccc(N4CCC(CN5CCC[C@@H](Nc6ccc7c(c6)C(=O)N(C6CCC(=O)NC6=O)C7=O)C5)CC4)nc3)CC2)cc1Cl] | N-((1r,3r)-3-(3-chloro-4-cyanophenoxy)- 2,2,4,4-tetramethylcyclobutyl)-6-(4-(((3R)-3- ((2-(2,6-dioxopiperidin-3-yl)-1,3- dioxoisoindolin-5-yl)amino)piperidin-1- yl)methyl)piperidin-1-yl)nicotinamide |
| GT-03400 | 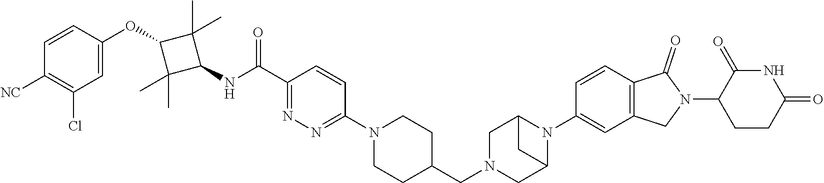 [SMILES: CC1(C)[C@H](NC(=O)c2ccc(N3CCC(CN4CC5CC(C4)N5c4ccc5c(c4)CN(C4CCC(=O)NC4=O)C5=O)CC3)nn2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((6-(2-(2,6- dioxopiperidin-3-yl)-1,3-dioxoisoindolin-5-yl)- 3,6-diazabicyclo[3.1.1]heptan-3- yl)methyl)piperidin-1-yl)pyridazine-3- carboxamide |
| GT-03401 | 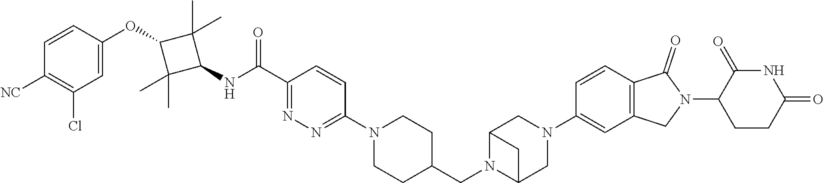 [SMILES: CC1(C)[C@H](NC(=O)c2ccc(N3CCC(CN4C5CC4CN(c4ccc6c(c4)CN(C4CCC(=O)NC4=O)C6=O)C5)CC3)nn2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((3-(2-(2,6- dioxopiperidin-3-yl)-1,3-dioxoisoindolin-5-yl)- 3,6-diazabicyclo[3.1.1]heptan-6- yl)methyl)piperidin-1-yl)pyridazine-3- carboxamide |
| GT-03399 | 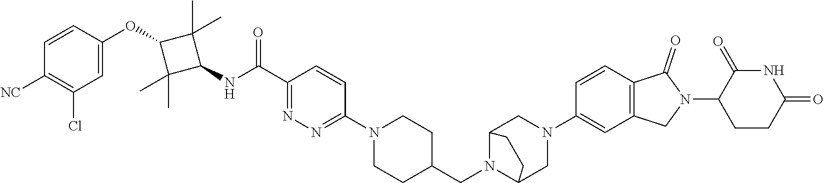 [SMILES: CC1(C)[C@H](NC(=O)c2ccc(N3CCC(CN4C5CCC4CN(c4ccc6c(c4)CN(C4CCC(=O)NC4=O)C6=O)C5)CC3)nn2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((3-(2-(2,6- dioxopiperidin-3-yl)-1,3-dioxoisoindolin-5-yl)- 3,8-diazabicyclo[3.2.1]octan-8- yl)methyl)piperidin-1-yl)pyridazine-3- carboxamide |
| GT-03398 | 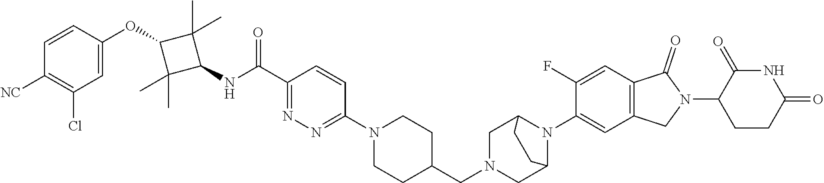 [SMILES: CC1(C)[C@H](NC(=O)c2ccc(N3CCC(CN4CC5CCC(C4)N5c4cc5c(cc4F)C(=O)N(C4CCC(=O)NC4=O)C5)CC3)nn2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((8-(2-(2,6- dioxopiperidin-3-yl)-6-fluoro-1,3- dioxoisoindolin-5-yl)-3,8- diazabicyclo[3.2.1]octan-3-yl)methyl)piperidin- 1-yl)pyridazine-3-carboxamide |
| | 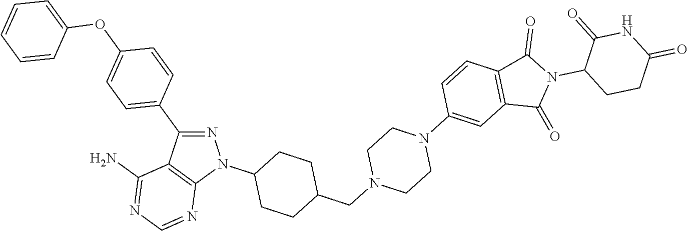 [SMILES: Nc1ncnc2c1c(-c1ccc(Oc3ccccc3)cc1)nn2C1CCC(CN2CCN(c3ccc4c(c3)C(=O)N(C3CCC(=O)NC3=O)C4=O)CC2)CC1] | 5-(4-((4-(4-amino-3-(4-phenoxyphenyl)-1H- pyrazolo[3,4-d]pyrimidin-1- yl)cyclohexyl)methyl)piperazin-1-yl)-2-(2,6- dioxopiperidin-3-yl)isoindoline-1,3-dione |
| | 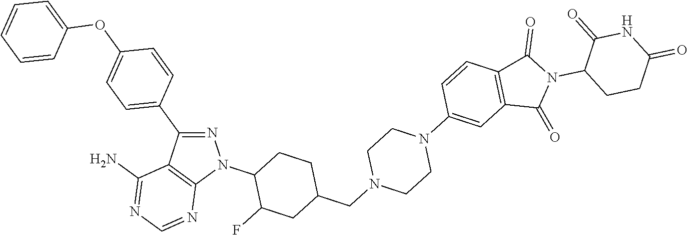 [SMILES: Nc1ncnc2c1c(-c1ccc(Oc3ccccc3)cc1)nn2C1CCC(CN2CCN(c3ccc4c(c3)C(=O)N(C3CCC(=O)NC3=O)C4=O)CC2)CC1F] | 5-(4-((4-(4-amino-3-(4-phenoxyphenyl)-1H- pyrazolo[3,4-d]pyrimidin-1-yl)-3- fluorocyclohexyl)methyl)piperazin-1-yl)-2- (2,6-dioxopiperidin-3-yl)isoindoline-1,3-dione |
| | 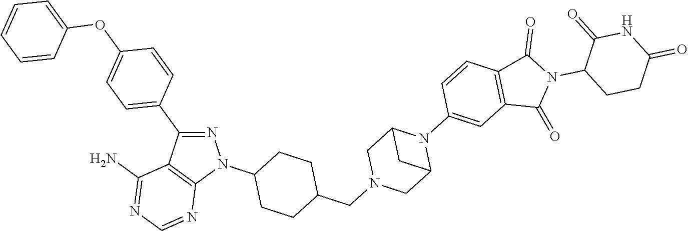 [SMILES: Nc1ncnc2c1c(-c1ccc(Oc3ccccc3)cc1)nn2C1CCC(CN2CC3CC(C2)N3c2ccc3c(c2)C(=O)N(C2CCC(=O)NC2=O)C3=O)CC1] | 5-(3-((4-(4-amino-3-(4-phenoxyphenyl)-1H- pyrazolo[3,4-d]pyrimidin-1- yl)cyclohexyl)methyl)-3,6- diazabicyclo[3.1.1]heptan-6-yl)-2-(2,6- dioxopiperidin-3-yl)isoindoline-1,3-dione |
| | 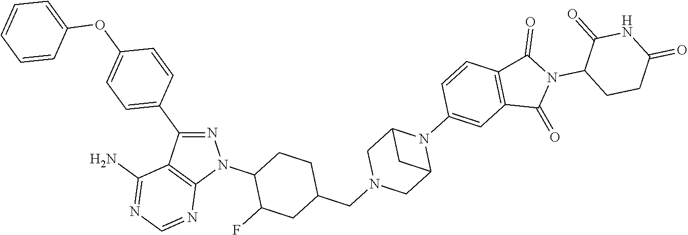 [SMILES: Nc1ncnc2c1c(-c1ccc(Oc3ccccc3)cc1)nn2C1CCC(CN2CC3CC(C2)N3c2ccc3c(c2)C(=O)N(C2CCC(=O)NC2=O)C3=O)CC1F] | 5-(3-((4-(4-amino-3-(4-phenoxyphenyl)-1H- pyrazolo[3,4-d]pyrimidin-1-yl)-3- fluorocyclohexyl)methyl)-3,6- diazabicyclo[3.1.1]heptan-6-yl)-2-(2,6- dioxopiperidin-3-yl)isoindoline-1,3-dione |
| | 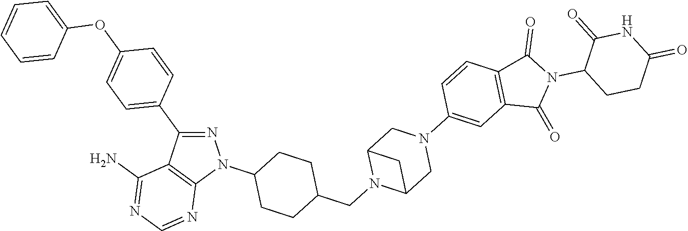 [SMILES: Nc1ncnc2c1c(-c1ccc(Oc3ccccc3)cc1)nn2C1CCC(CN2C3CC2CN(c2ccc4c(c2)C(=O)N(C2CCC(=O)NC2=O)C4=O)C3)CC1] | 5-(6-((4-(4-amino-3-(4-phenoxyphenyl)-1H- pyrazolo[3,4-d]pyrimidin-1- yl)cyclohexyl)methyl)-3,6- diazabicyclo[3.1.1]heptan-3-yl)-2-(2,6- dioxopiperidin-3-yl)isoindoline-1,3-dione |
| | 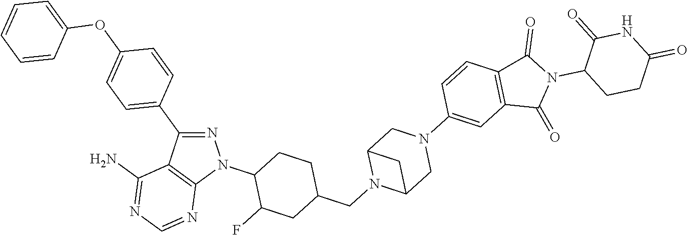 [SMILES: Nc1ncnc2c1c(-c1ccc(Oc3ccccc3)cc1)nn2C1CCC(CN2C3CC2CN(c2ccc4c(c2)C(=O)N(C2CCC(=O)NC2=O)C4=O)C3)CC1F] | 5-(6-((4-(4-amino-3-(4-phenoxyphenyl)-1H- pyrazolo[3,4-d]pyrimidin-1-yl)-3- fluorocyclohexyl)methyl)-3,6- diazabicyclo[3.1.1]heptan-3-yl)-2-(2,6- dioxopiperidin-3-yl)isoindoline-1,3-dione |
| | 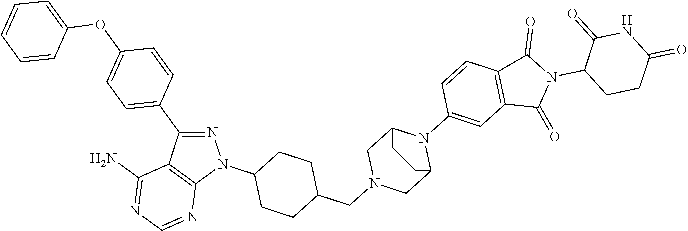 [SMILES: Nc1ncnc2c1c(-c1ccc(Oc3ccccc3)cc1)nn2C1CCC(CN2CC3CCC(C2)N3c2ccc3c(c2)C(=O)N(C2CCC(=O)NC2=O)C3=O)CC1] | 5-(3-((4-(4-amino-3-(4-phenoxyphenyl)-1H- pyrazolo[3,4-d]pyrimidin-1- yl)cyclohexyl)methyl)-3,8- diazabicyclo[3.2.1]octan-8-yl)-2-(2,6- dioxopiperidin-3-yl)isoindoline-1,3-dione |
| | 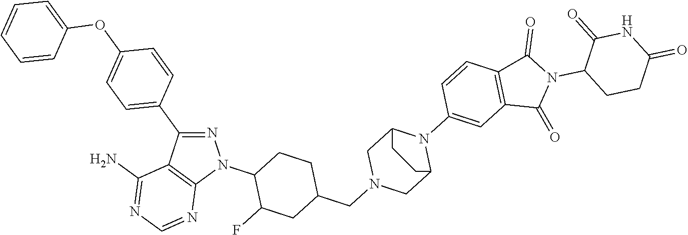 [SMILES: Nc1ncnc2c1c(-c1ccc(Oc3ccccc3)cc1)nn2C1CCC(CN2CC3CCC(C2)N3c2ccc3c(c2)C(=O)N(C2CCC(=O)NC2=O)C3=O)CC1F] | 5-(3-((4-(4-amino-3-(4-phenoxyphenyl)-1H- pyrazolo[3,4-d]pyrimidin-1-yl)-3- fluorocyclohexyl)methyl)-3,8- diazabicyclo[3.2.1]octan-8-yl)-2-(2,6- dioxopiperidin-3-yl)isoindoline-1,3-dione |
| | 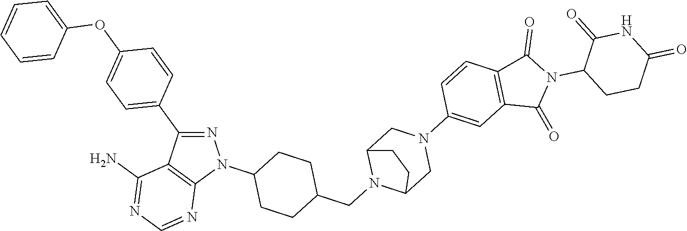 [SMILES: Nc1ncnc2c1c(-c1ccc(Oc3ccccc3)cc1)nn2C1CCC(CN2C3CCC2CN(c2ccc4c(c2)C(=O)N(C2CCC(=O)NC2=O)C4=O)C3)CC1] | 5-(8-((4-(4-amino-3-(4-phenoxyphenyl)-1H- pyrazolo[3,4-d]pyrimidin-1- yl)cyclohexyl)methyl)-3,8- diazabicyclo[3.2.1]octan-3-yl)-2-(2,6- dioxopiperidin-3-yl)isoindoline-1,3-dione |
| | 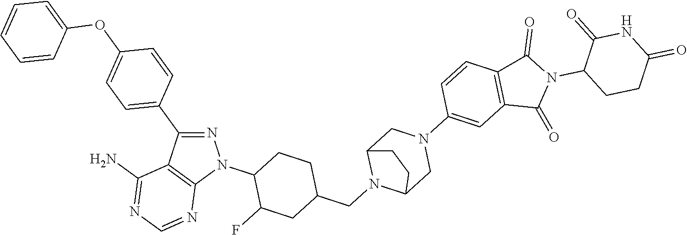 [SMILES: Nc1ncnc2c1c(-c1ccc(Oc3ccccc3)cc1)nn2C1CCC(CN2C3CCC2CN(c2ccc4c(c2)C(=O)N(C2CCC(=O)NC2=O)C4=O)C3)CC1F] | 5-(8-((4-(4-amino-3-(4-phenoxyphenyl)-1H- pyrazolo[3,4-d]pyrimidin-1-yl)-3- fluorocyclohexyl)methyl)-3,8- diazabicyclo[3.2.1]octan-3-yl)-2-(2,6- dioxopiperidin-3-yl)isoindoline-1,3-dione |
| | 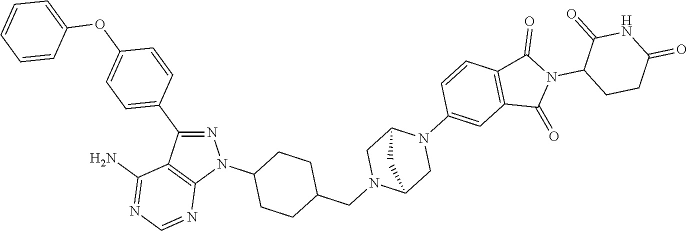 [SMILES: Nc1ncnc2c1c(-c1ccc(Oc3ccccc3)cc1)nn2C1CCC(CN2C[C@@H]3C[C@H]2CN3c2ccc3c(c2)C(=O)N(C2CCC(=O)NC2=O)C3=O)CC1] | 5-((1S,4S)-5-((4-(4-amino-3-(4- phenoxyphenyl)-1H-pyrazolo[3,4-d]pyrimidin- 1-yl)cyclohexyl)methyl)-2,5- diazabicyclo[2.2.1]heptan-2-yl)-2-(2,6- dioxopiperidin-3-yl)isoindoline-1,3-dione |
| | 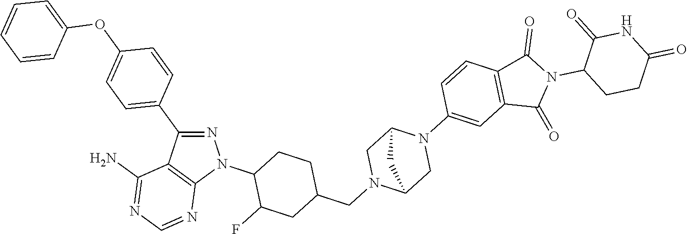 [SMILES: Nc1ncnc2c1c(-c1ccc(Oc3ccccc3)cc1)nn2C1CCC(CN2C[C@@H]3C[C@H]2CN3c2ccc3c(c2)C(=O)N(C2CCC(=O)NC2=O)C3=O)CC1F] | 5-((1S,4S)-5-((4-(4-amino-3-(4- phenoxyphenyl)-1H-pyrazolo[3,4-d]pyrimidin- 1-yl)-3-fluorocyclohexyl)methyl)-2,5- diazabicyclo[2.2.1]heptan-2-yl)-2-(2,6- dioxopiperidin-3-yl)isoindoline-1,3-dione |
| | 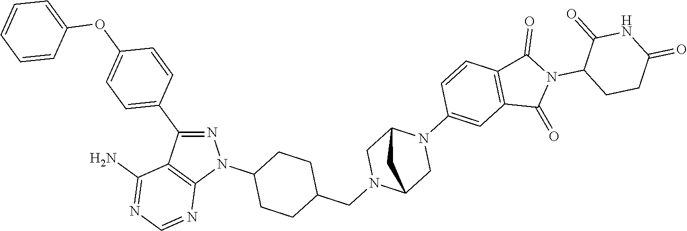 [SMILES: Nc1ncnc2c1c(-c1ccc(Oc3ccccc3)cc1)nn2C1CCC(CN2C[C@H]3C[C@@H]2CN3c2ccc3c(c2)C(=O)N(C2CCC(=O)NC2=O)C3=O)CC1] | 5-((1R,4R)-5-((4-(4-amino-3-(4- phenoxyphenyl)-1H-pyrazolo[3,4-d]pyrimidin- 1-yl)cyclohexyl)methyl)-2,5- diazabicyclo[2.2.1]heptan-2-yl)-2-(2,6- dioxopiperidin-3-yl)isoindoline-1,3-dione |
| | 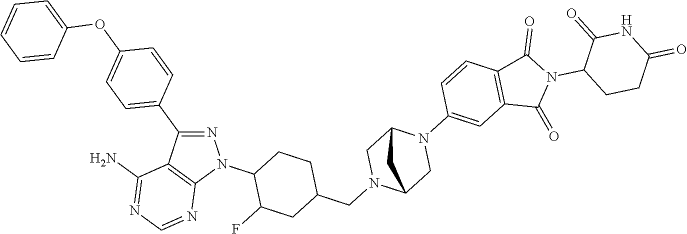 [SMILES: Nc1ncnc2c1c(-c1ccc(Oc3ccccc3)cc1)nn2C1CCC(CN2C[C@H]3C[C@@H]2CN3c2ccc3c(c2)C(=O)N(C2CCC(=O)NC2=O)C3=O)CC1F] | 5-((1R,4R)-5-((4-(4-amino-3-(4- phenoxyphenyl)-1H-pyrazolo[3,4-d]pyrimidin- 1-yl)-3-fluorocyclohexyl)methyl)-2,5- diazabicyclo[2.2.1]heptan-2-yl)-2-(2,6- dioxopiperidin-3-yl)isoindoline-1,3-dione |
| | 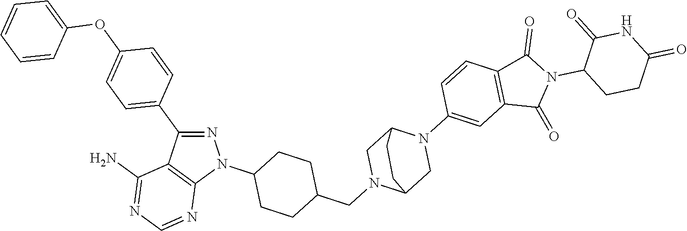 [SMILES: Nc1ncnc2c1c(-c1ccc(Oc3ccccc3)cc1)nn2C1CCC(CN2CC3CCC2CN3c2ccc3c(c2)C(=O)N(C2CCC(=O)NC2=O)C3=O)CC1] | 5-(5-((4-(4-amino-3-(4-phenoxyphenyl)-1H- pyrazolo[3,4-d]pyrimidin-1- yl)cyclohexyl)methyl)-2,5- diazabicyclo[2.2.2]octan-2-yl)-2-(2,6- dioxopiperidin-3-yl)isoindoline-1,3-dione |
| | 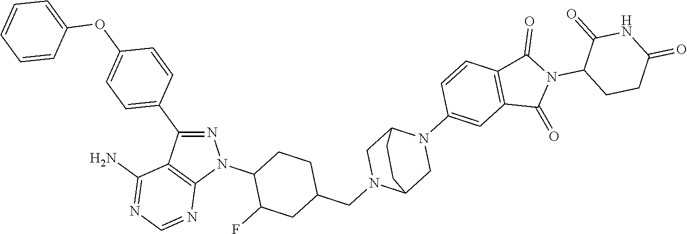 [SMILES: Nc1ncnc2c1c(-c1ccc(Oc3ccccc3)cc1)nn2C1CCC(CN2CC3CCC2CN3c2ccc3c(c2)C(=O)N(C2CCC(=O)NC2=O)C3=O)CC1F] | 5-(5-((4-(4-amino-3-(4-phenoxyphenyl)-1H- pyrazolo[3,4-d]pyrimidin-1-yl)-3- fluorocyclohexyl)methyl)-2,5- diazabicyclo[2.2.2]octan-2-yl)-2-(2,6- dioxopiperidin-3-yl)isoindoline-1,3-dione |
| | 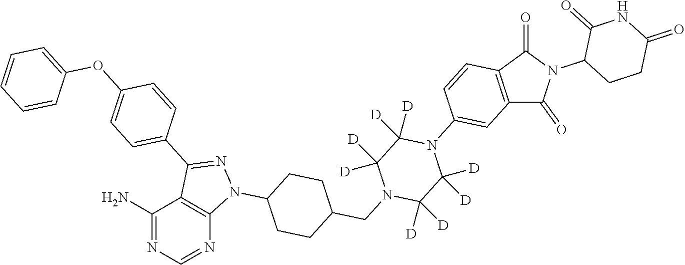 [SMILES: [2H]C1([2H])N(CC2CCC(n3nc(-c4ccc(Oc5ccccc5)cc4)c4c(N)ncnc43)CC2)C([2H])([2H])C([2H])([2H])N(c2ccc3c(c2)C(=O)N(C2CCC(=O)NC2=O)C3=O)C1([2H])[2H]] | 5-(4-((4-(4-amino-3-(4-phenoxyphenyl)-1H- pyrazolo[3,4-d]pyrimidin-1- yl)cyclohexyl)methyl)piperazin-1-yl- 2,2,3,3,5,5,6,6-d₈)-2-(2,6-dioxopiperidin-3- yl)isoindoline-1,3-dione |
| | 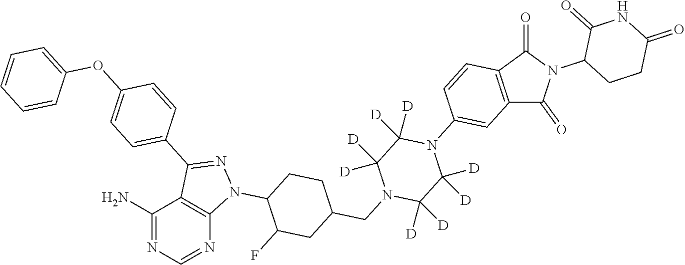 [SMILES: [2H]C1([2H])N(CC2CCC(n3nc(-c4ccc(Oc5ccccc5)cc4)c4c(N)ncnc43)C(F)C2)C([2H])([2H])C([2H])([2H])N(c2ccc3c(c2)C(=O)N(C2CCC(=O)NC2=O)C3=O)C1([2H])[2H]] | 5-(4-((4-(4-amino-3-(4-phenoxyphenyl)-1H- pyrazolo[3,4-d]pyrimidin-1-yl)-3- fluorocyclohexyl)methyl)piperazin-1-yl- 2,2,3,3,5,5,6,6-d₈)-2-(2,6-dioxopiperidin-3- yl)isoindoline-1,3-dione |
| | 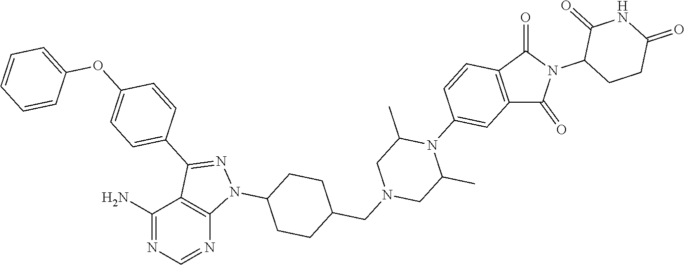 [SMILES: CC1CN(CC2CCC(n3nc(-c4ccc(Oc5ccccc5)cc4)c4c(N)ncnc43)CC2)CC(C)N1c1ccc2c(c1)C(=O)N(C1CCC(=O)NC1=O)C2=O] | 5-(4-((4-(4-amino-3-(4-phenoxyphenyl)-1H- pyrazolo[3,4-d]pyrimidin-1- yl)cyclohexyl)methyl)-2,6-dimethylpiperazin- 1-yl)-2-(2,6-dioxopiperidin-3-yl)isoindoline- 1,3-dione |
| | 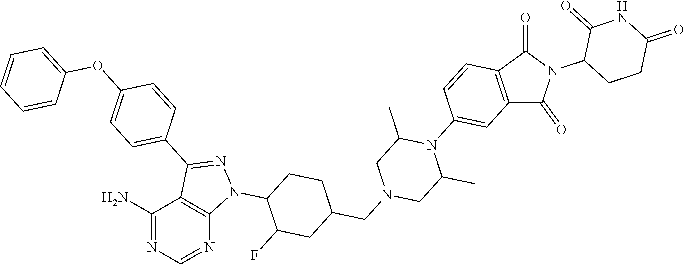 [SMILES: CC1CN(CC2CCC(n3nc(-c4ccc(Oc5ccccc5)cc4)c4c(N)ncnc43)C(F)C2)CC(C)N1c1ccc2c(c1)C(=O)N(C1CCC(=O)NC1=O)C2=O] | 5-(4-((4-(4-amino-3-(4-phenoxyphenyl)-1H- pyrazolo[3,4-d]pyrimidin-1-yl)-3- fluorocyclohexyl)methyl)-2,6- dimethylpiperazin-1-yl)-2-(2,6-dioxopiperidin- 3-yl)isoindoline-1,3-dione |
| | 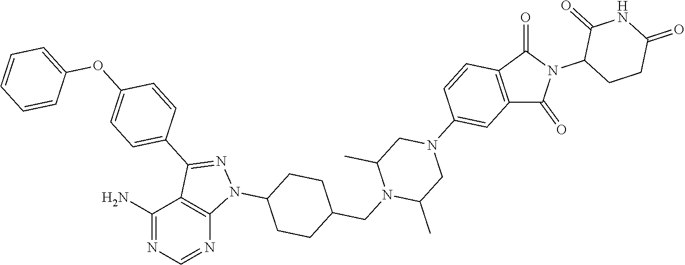 [SMILES: CC1CN(c2ccc3c(c2)C(=O)N(C2CCC(=O)NC2=O)C3=O)CC(C)N1CC1CCC(n2nc(-c3ccc(Oc4ccccc4)cc3)c3c(N)ncnc32)CC1] | 5-(4-((4-(4-amino-3-(4-phenoxyphenyl)-1H- pyrazolo[3,4-d]pyrimidin-1- yl)cyclohexyl)methyl)-3,5-dimethylpiperazin- 1-yl)-2-(2,6-dioxopiperidin-3-yl)isoindoline- 1,3-dione |
| | 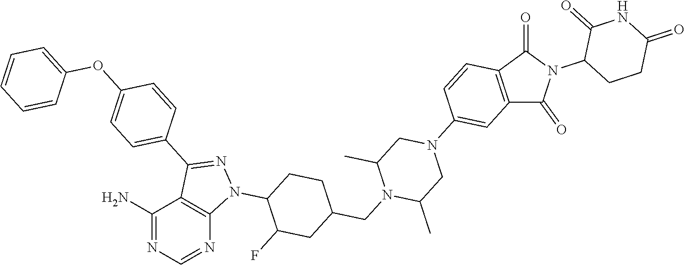 [SMILES: CC1CN(c2ccc3c(c2)C(=O)N(C2CCC(=O)NC2=O)C3=O)CC(C)N1CC1CCC(n2nc(-c3ccc(Oc4ccccc4)cc3)c3c(N)ncnc32)C(F)C1] | 5-(4-((4-(4-amino-3-(4-phenoxyphenyl)-1H- pyrazolo[3,4-d]pyrimidin-1-yl)-3- fluorocyclohexyl)methyl)-3,5- dimethylpiperazin-1-yl)-2-(2,6-dioxopiperidin- 3-yl)isoindoline-1,3-dione |
| | 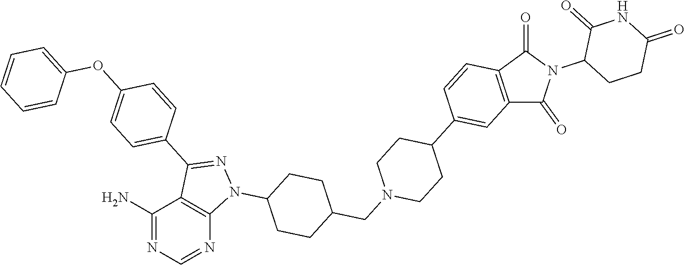 [SMILES: Nc1ncnc2c1c(-c1ccc(Oc3ccccc3)cc1)nn2C1CCC(CN2CCC(c3ccc4c(c3)C(=O)N(C3CCC(=O)NC3=O)C4=O)CC2)CC1] | 5-(1-((4-(4-amino-3-(4-phenoxyphenyl)-1H- pyrazolo[3,4-d]pyrimidin-1 - yl)cyclohexyl)methyl)piperidin-4-yl)-2-(2,6- dioxopiperidin-3-yl)isoindoline-1,3-dione |
| | 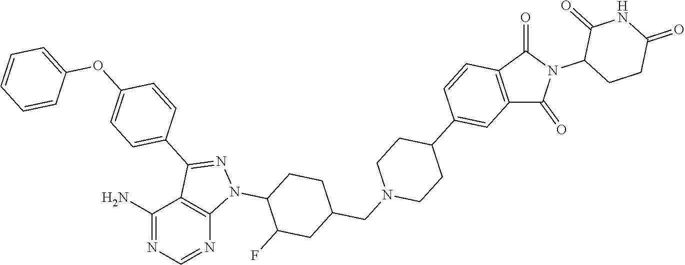 [SMILES: Nc1ncnc2c1c(-c1ccc(Oc3ccccc3)cc1)nn2C1CCC(CN2CCC(c3ccc4c(c3)C(=O)N(C3CCC(=O)NC3=O)C4=O)CC2)CC1F] | 5-(1-((4-(4-amino-3-(4-phenoxyphenyl)-1H- pyrazolo[3,4-d]pyrimidin-1-yl)-3- fluorocyclohexyl)methyl)piperidin-4-yl)-2- (2,6-dioxopiperidin-3-yl)isoindoline-1,3-dione |
| | 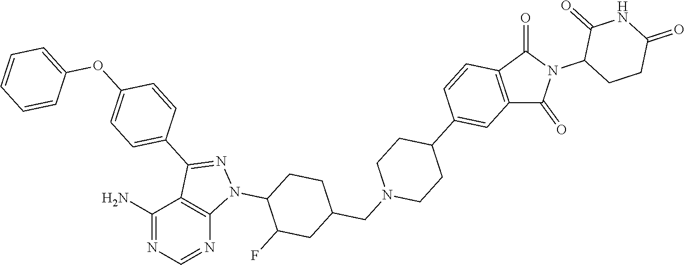 [SMILES: Nc1ncnc2c1c(-c1ccc(Oc3ccccc3)cc1)nn2C1CCC(CN2CCC(c3ccc4c(c3)C(=O)N(C3CCC(=O)NC3=O)C4=O)CC2)CC1F] | 5-(4-((4-(4-amino-3-(4-phenoxyphenyl)-1H- pyrazolo[3,4-d]pyrimidin-1- yl)cyclohexyl)methyl)piperazin-1-yl)-2-(2,6- dioxopiperidin-3-yl)-6-fluoroisoindoline-1,3- dione |
| | 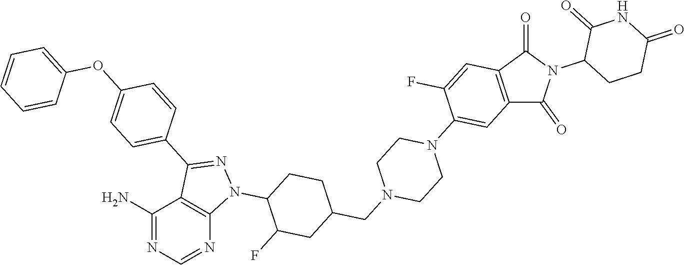 [SMILES: Nc1ncnc2c1c(-c1ccc(Oc3ccccc3)cc1)nn2C1CCC(CN2CCN(c3cc4c(cc3F)C(=O)N(C3CCC(=O)NC3=O)C4=O)CC2)CC1F] | 5-(4-((4-(4-amino-3-(4-phenoxyphenyl)-1H- pyrazolo[3,4-d]pyrimidin-1-yl)-3- fluorocyclohexyl)methyl)piperazin-1-yl)-2- (2,6-dioxopiperidin-3-yl)-6-fluoroisoindoline- 1,3-dione |
| | 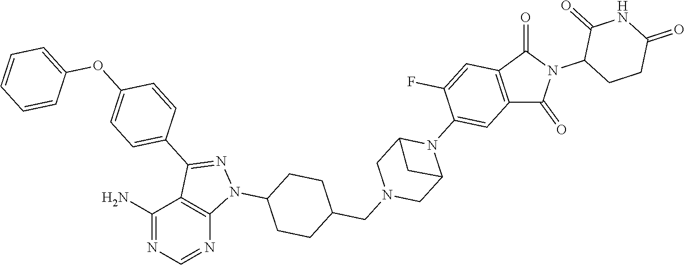 [SMILES: Nc1ncnc2c1c(-c1ccc(Oc3ccccc3)cc1)nn2C1CCC(CN2CC3CC(C2)N3c2cc3c(cc2F)C(=O)N(C2CCC(=O)NC2=O)C3=O)CC1] | 5-(3-((4-(4-amino-3-(4-phenoxyphenyl)-1H- pyrazolo[3,4-d]pyrimidin-1- yl)cyclohexyl)methyl)-3,6- diazabicyclo[3.1.1]heptan-6-yl)-2-(2,6- dioxopiperidin-3-yl)-6-fluoroisoindoline-1,3- dione |
| | 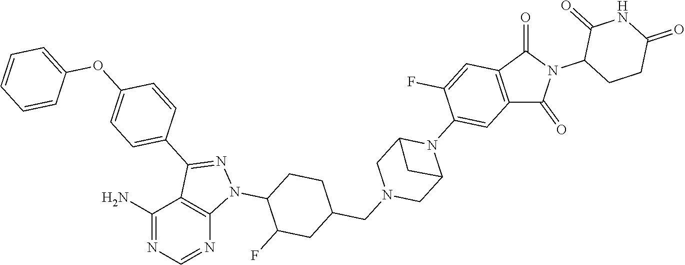 [SMILES: Nc1ncnc2c1c(-c1ccc(Oc3ccccc3)cc1)nn2C1CCC(CN2CC3CC(C2)N3c2cc3c(cc2F)C(=O)N(C2CCC(=O)NC2=O)C3=O)CC1F] | 5-(3-((4-(4-amino-3-(4-phenoxyphenyl)-1H- pyrazolo[3,4-d]pyrimidin-1-yl)-3- fluorocyclohexyl)methyl)-3,6- diazabicyclo[3.1.1]heptan-6-yl)-2-(2,6- dioxopiperidin-3-yl)-6-fluoroisoindoline-1,3- dione |
| | 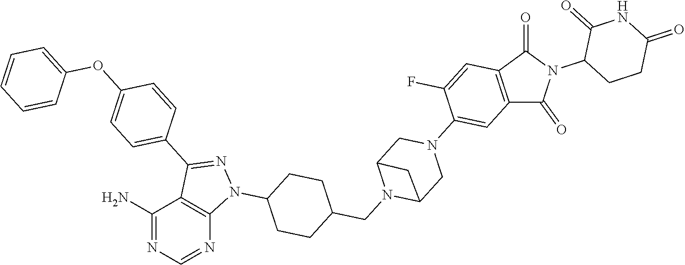 [SMILES: Nc1ncnc2c1c(-c1ccc(Oc3ccccc3)cc1)nn2C1CCC(CN2C3CC2CN(c2cc4c(cc2F)C(=O)N(C2CCC(=O)NC2=O)C4=O)C3)CC1] | 5-(6-((4-(4-amino-3-(4-phenoxyphenyl)-1H- pyrazolo[3,4-d]pyrimidin-1- yl)cyclohexyl)methyl)-3,6- diazabicyclo[3.1.1]heptan-3-yl)-2-(2,6- dioxopiperidin-3-yl)-6-fluoroisoindoline-1,3- dione |
| | 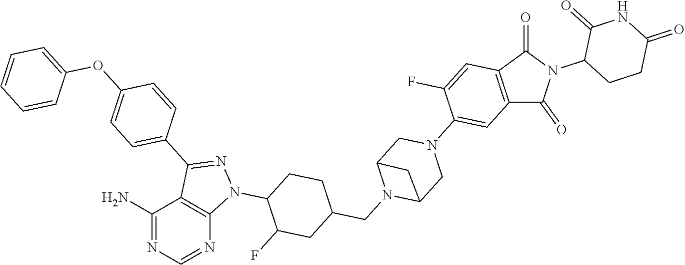 [SMILES: Nc1ncnc2c1c(-c1ccc(Oc3ccccc3)cc1)nn2C1CCC(CN2C3CC2CN(c2cc4c(cc2F)C(=O)N(C2CCC(=O)NC2=O)C4=O)C3)CC1F] | 5-(6-((4-(4-amino-3-(4-phenoxyphenyl)-1H- pyrazolo[3,4-d]pyrimidin-1-yl)-3- fluorocyclohexyl)methyl)-3,6- diazabicyclo[3.1.1]heptan-3-yl)-2-(2,6- dioxopiperidin-3-yl)-6-fluoroisoindoline-1,3- dione |
| | 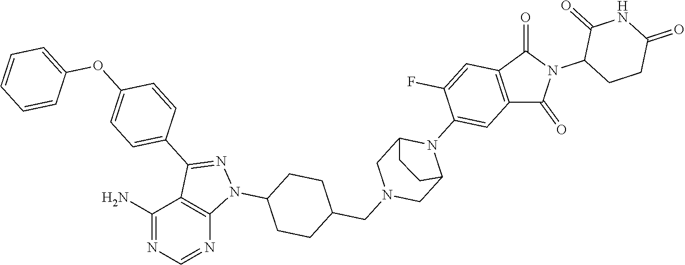 [SMILES: Nc1ncnc2c1c(-c1ccc(Oc3ccccc3)cc1)nn2C1CCC(CN2CC3CCC(C2)N3c2cc3c(cc2F)C(=O)N(C2CCC(=O)NC2=O)C3=O)CC1] | 5-(3-((4-(4-amino-3-(4-phenoxyphenyl)-1H- pyrazolo[3,4-d]pyrimidin-1- yl)cyclohexyl)methyl)-3,8- diazabicyclo[3.2.1]octan-8-yl)-2-(2,6- dioxopiperidin-3-yl)-6-fluoroisoindoline-1,3- dione |
| | 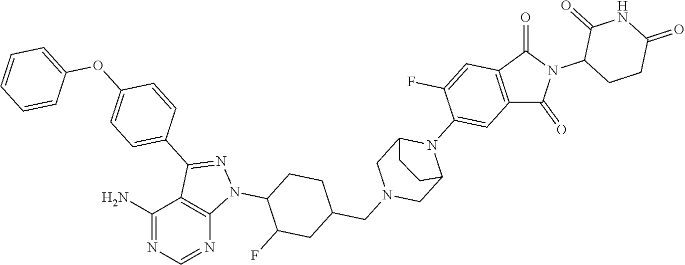 [SMILES: Nc1ncnc2c1c(-c1ccc(Oc3ccccc3)cc1)nn2C1CCC(CN2CC3CCC(C2)N3c2cc3c(cc2F)C(=O)N(C2CCC(=O)NC2=O)C3=O)CC1F] | 5-(3-((4-(4-amino-3-(4-phenoxyphenyl)-1H- pyrazolo[3,4-d]pyrimidin-1-yl)-3- fluorocyclohexyl)methyl)-3,8- diazabicyclo[3.2.1]octan-8-yl)-2-(2,6- dioxopiperidin-3-yl)-6-fluoroisoindoline-1,3- dione |
| | 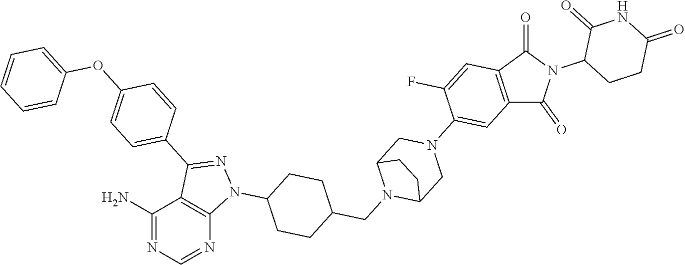 [SMILES: Nc1ncnc2c1c(-c1ccc(Oc3ccccc3)cc1)nn2C1CCC(CN2C3CCC2CN(c2cc4c(cc2F)C(=O)N(C2CCC(=O)NC2=O)C4=O)C3)CC1] | 5-(8-((4-(4-amino-3-(4-phenoxyphenyl)-1H- pyrazolo[3,4-d]pyrimidin-1- yl)cyclohexyl)methyl)-3,8- diazabicyclo[3.2.1]octan-3-yl)-2-(2,6- dioxopiperidin-3-yl)-6-fluoroisoindoline-1,3- dione |
| | 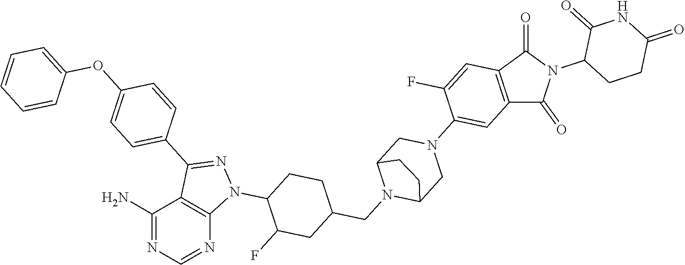 [SMILES: Nc1ncnc2c1c(-c1ccc(Oc3ccccc3)cc1)nn2C1CCC(CN2C3CCC2CN(c2cc4c(cc2F)C(=O)N(C2CCC(=O)NC2=O)C4=O)C3)CC1F] | 5-(8-((4-(4-amino-3-(4-phenoxyphenyl)-1H- pyrazolo[3,4-d]pyrimidin-1-yl)-3- fluorocyclohexyl)methyl)-3,8- diazabicyclo[3.2.1]octan-3-yl)-2-(2,6- dioxopiperidin-3-yl)-6-fluoroisoindoline-1,3- dione |
| | 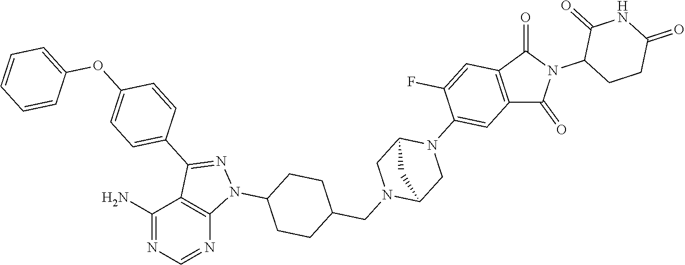 [SMILES: Nc1ncnc2c1c(-c1ccc(Oc3ccccc3)cc1)nn2C1CCC(CN2C[C@@H]3C[C@H]2CN3c2cc3c(cc2F)C(=O)N(C2CCC(=O)NC2=O)C3=O)CC1] | 5-((1S,4S)-5-((4-(4-amino-3-(4- phenoxyphenyl)-1H-pyrazolo[3,4-d]pyrimidin- 1-yl)cyclohexyl)methyl)-2,5- diazabicyclo[2.2.1]heptan-2-yl)-2-(2,6- dioxopiperidin-3-yl)-6-fluoroisoindoline-1,3- dione |
| | 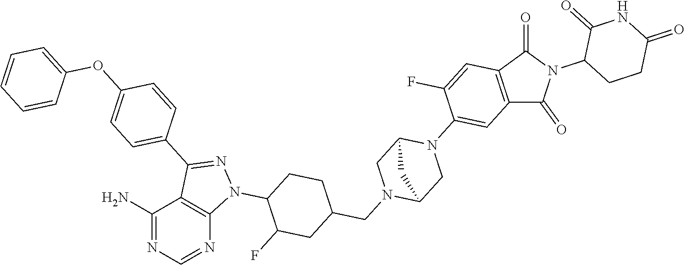 [SMILES: Nc1ncnc2c1c(-c1ccc(Oc3ccccc3)cc1)nn2C1CCC(CN2C[C@@H]3C[C@H]2CN3c2cc3c(cc2F)C(=O)N(C2CCC(=O)NC2=O)C3=O)CC1F] | 5-((1S,4S)-5-((4-(4-amino-3-(4- phenoxyphenyl)-1H-pyrazolo[3,4-d]pyrimidin- 1-yl)-3-fluorocyclohexyl)methyl)-2,5- diazabicyclo[2.2.1]heptan-2-yl)-2-(2,6- dioxopiperidin-3-yl)-6-fluoroisoindoline-1,3- dione |
| | 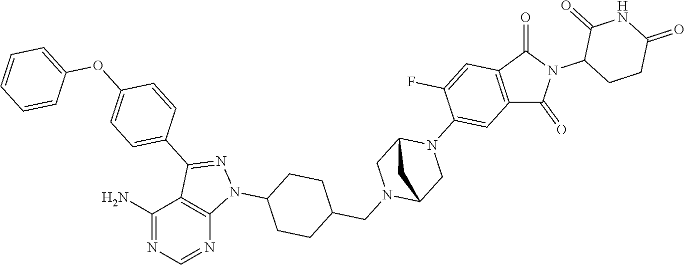 [SMILES: Nc1ncnc2c1c(-c1ccc(Oc3ccccc3)cc1)nn2C1CCC(CN2C[C@H]3C[C@@H]2CN3c2cc3c(cc2F)C(=O)N(C2CCC(=O)NC2=O)C3=O)CC1] | 5-((1R,4R)-5-((4-(4-amino-3-(4- phenoxyphenyl)-1H-pyrazolo[3,4-d]pyrimidin- 1-yl)cyclohexyl)methyl)-2,5- diazabicyclo[2.2.1]heptan-2-yl)-2-(2,6- dioxopiperidin-3-yl)-6-fluoroisoindoline-1,3- dione |
| | 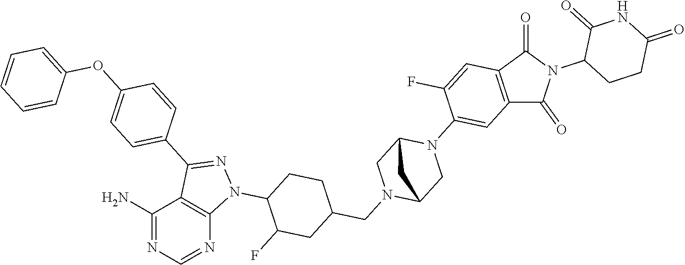 [SMILES: Nc1ncnc2c1c(-c1ccc(Oc3ccccc3)cc1)nn2C1CCC(CN2C[C@H]3C[C@@H]2CN3c2cc3c(cc2F)C(=O)N(C2CCC(=O)NC2=O)C3=O)CC1F] | 5-((1R,4R)-5-((4-(4-amino-3-(4- phenoxyphenyl)-1H-pyrazolo[3,4-d]pyrimidin- 1-yl)-3-fluorocyclohexyl)methyl)-2,5- diazabicyclo[2.2.1]heptan-2-yl)-2-(2,6- dioxopiperidin-3-yl)-6-fluoroisoindoline-1,3- dione |
| | 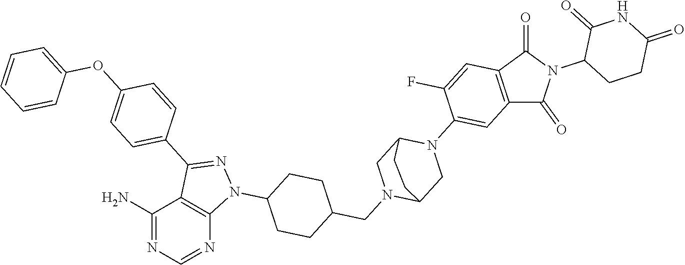 [SMILES: Nc1ncnc2c1c(-c1ccc(Oc3ccccc3)cc1)nn2C1CCC(CN2CC3CCC2CN3c2cc3c(cc2F)C(=O)N(C2CCC(=O)NC2=O)C3=O)CC1] | 5-(5-((4-(4-amino-3-(4-phenoxyphenyl)-1H- pyrazolo[3,4-d]pyrimidin-1- yl)cyclohexyl)methyl)-2,5- diazabicyclo[2.2.2]octan-2-yl)-2-(2,6- dioxopiperidin-3-yl)-6-fluoroisoindoline-1,3- dione |
| | 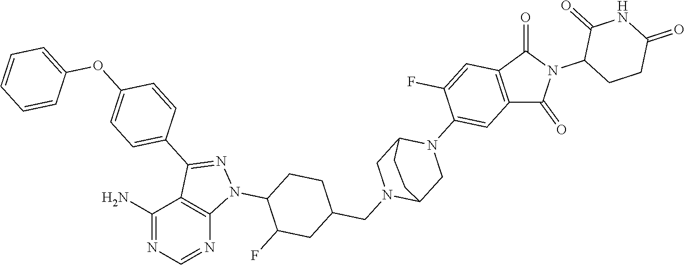 [SMILES: Nc1ncnc2c1c(-c1ccc(Oc3ccccc3)cc1)nn2C1CCC(CN2CC3CCC2CN3c2cc3c(cc2F)C(=O)N(C2CCC(=O)NC2=O)C3=O)CC1F] | 5-(5-((4-(4-amino-3-(4-phenoxyphenyl)-1H- pyrazolo[3,4-d]pyrimidin-1-yl)-3- fluorocyclohexyl)methyl)-2,5- diazabicyclo[2.2.2]octan-2-yl)-2-(2,6- dioxopiperidin-3-yl)-6-fluoroisoindoline-1,3- dione |
| | 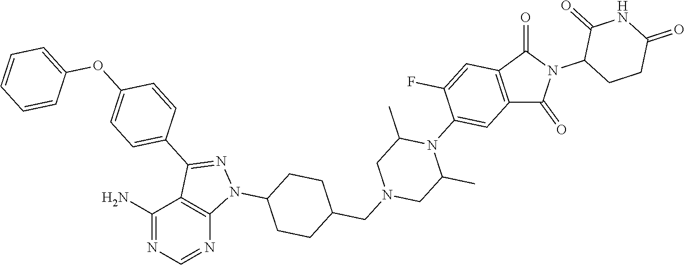 [SMILES: CC1CN(CC2CCC(n3nc(-c4ccc(Oc5ccccc5)cc4)c4c(N)ncnc43)CC2)CC(C)N1c1cc2c(cc1F)C(=O)N(C1CCC(=O)NC1=O)C2=O] | 5-(4-((4-(4-amino-3-(4-phenoxyphenyl)-1H- pyrazolo[3,4-d]pyrimidin-1- yl)cyclohexyl)methyl)-2,6-dimethylpiperazin- 1-yl)-2-(2,6-dioxopiperidin-3-yl)-6- fluoroisoindoline-1,3-dione |
| | 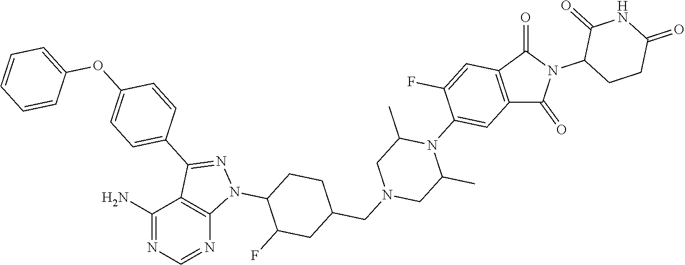 [SMILES: CC1CN(CC2CCC(n3nc(-c4ccc(Oc5ccccc5)cc4)c4c(N)ncnc43)C(F)C2)CC(C)N1c1cc2c(cc1F)C(=O)N(C1CCC(=O)NC1=O)C2=O] | 5-(4-((4-(4-amino-3-(4-phenoxyphenyl)-1H- pyrazolo[3,4-d]pyrimidin-1-yl)-3- fluorocyclohexyl)methyl)-2,6- dimethylpiperazin-1-yl)-2-(2,6-dioxopiperidin- 3-yl)-6-fluoroisoindoline-1,3-dione |
| | 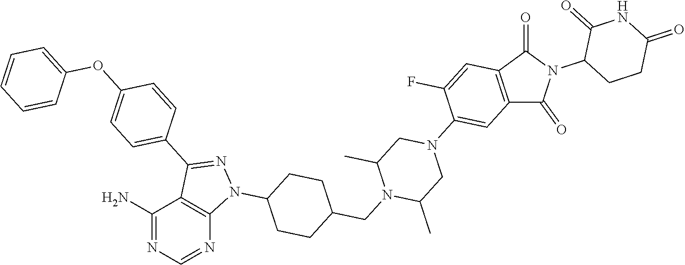 [SMILES: CC1CN(c2cc3c(cc2F)C(=O)N(C2CCC(=O)NC2=O)C3=O)CC(C)N1CC1CCC(n2nc(-c3ccc(Oc4ccccc4)cc3)c3c(N)ncnc32)CC1] | 5-(4-((4-(4-amino-3-(4-phenoxyphenyl)-1H- pyrazolo[3,4-d]pyrimidin-1- yl)cyclohexyl)methyl)-3,5-dimethylpiperazin- 1-yl)-2-(2,6-dioxopiperidin-3-yl)-6- fluoroisoindoline-1,3-dione |
| | 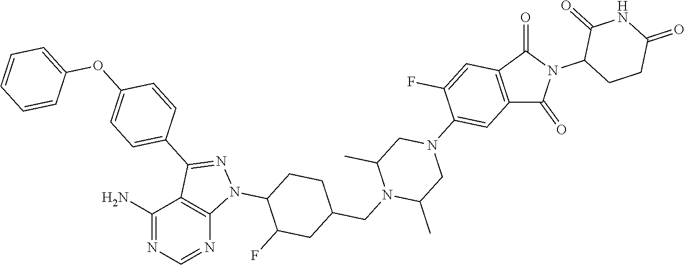 [SMILES: CC1CN(c2cc3c(cc2F)C(=O)N(C2CCC(=O)NC2=O)C3=O)CC(C)N1CC1CCC(n2nc(-c3ccc(Oc4ccccc4)cc3)c3c(N)ncnc32)C(F)C1] | 5-(4-((4-(4-amino-3-(4-phenoxyphenyl)-1H- pyrazolo[3,4-d]pyrimidin-1-yl)-3- fluorocyclohexyl)methyl)-3,5- dimethylpiperazin-1-yl)-2-(2,6-dioxopiperidin- 3-yl)-6-fluoroisoindoline-1,3-dione |
| | 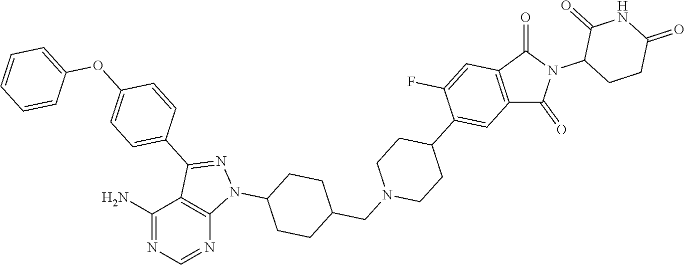 [SMILES: Nc1ncnc2c1c(-c1ccc(Oc3ccccc3)cc1)nn2C1CCC(CN2CCC(c3cc4c(cc3F)C(=O)N(C3CCC(=O)NC3=O)C4=O)CC2)CC1] | 5-(1-((4-(4-amino-3-(4-phenoxyphenyl)-1H- pyrazolo[3,4-d]pyrimidin-1- yl)cyclohexyl)methyl)piperidin-4-yl)-2-(2,6- dioxopiperidin-3-yl)-6-fluoroisoindoline-1,3- dione |
| | 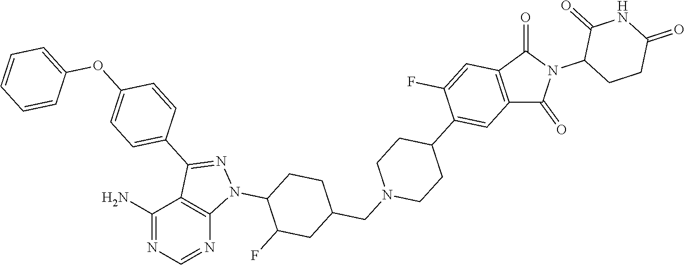 [SMILES: Nc1ncnc2c1c(-c1ccc(Oc3ccccc3)cc1)nn2C1CCC(CN2CCC(c3cc4c(cc3F)C(=O)N(C3CCC(=O)NC3=O)C4=O)CC2)CC1F] | 5-(1-((4-(4-amino-3-(4-phenoxyphenyl)-1H- pyrazolo[3,4-d]pyrimidin-1-yl)-3- fluorocyclohexyl)methyl)piperidin-4-yl)-2- (2,6-dioxopiperidin-3-yl)-6-fluoroisoindoline- 1,3-dione |
| | 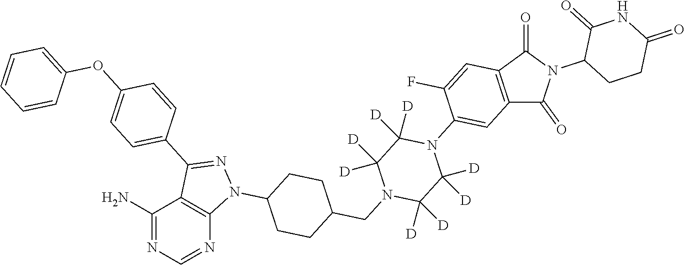 [SMILES: [2H]C1([2H])N(CC2CCC(n3nc(-c4ccc(Oc5ccccc5)cc4)c4c(N)ncnc43)CC2)C([2H])([2H])C([2H])([2H])N(c2cc3c(cc2F)C(=O)N(C2CCC(=O)NC2=O)C3=O)C1([2H])[2H]] | 5-(4-((4-(4-amino-3-(4-phenoxyphenyl)-1H- pyrazolo [3,4-d]pyrimidin-1- yl)cyclohexyl)methyl)piperazin-1-yl- 2,2,3,3,5,5,6,6-d₈)-2-(2,6-dioxopiperidin-3-yl)- 6-fluoroisoindoline-1,3-dione |
| | 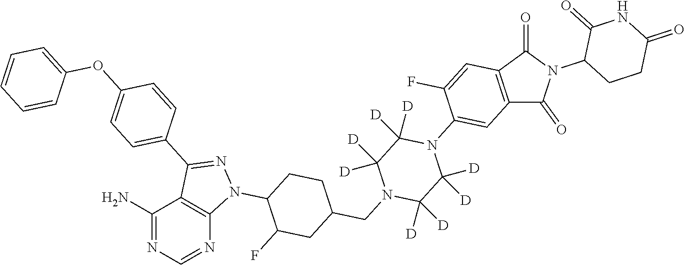 [SMILES: [2H]C1([2H])N(CC2CCC(n3nc(-c4ccc(Oc5ccccc5)cc4)c4c(N)ncnc43)C(F)C2)C([2H])([2H])C([2H])([2H])N(c2cc3c(cc2F)C(=O)N(C2CCC(=O)NC2=O)C3=O)C1([2H])[2H]] | 5-(4-((4-(4-amino-3-(4-phenoxyphenyl)-1H- pyrazolo[3,4-d]pyrimidin-1-yl)-3- fluorocyclohexyl)methyl)piperazin-1-yl- 2,2,3,3,5,5,6,6-d₈)-2-(2,6-dioxopiperidin-3-yl)- 6-fluoroisoindoline-1,3-dione |
| | 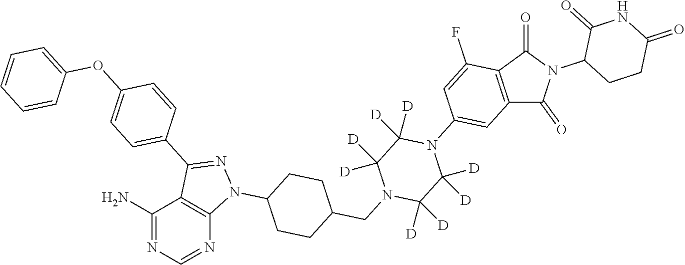 [SMILES: [2H]C1([2H])N(CC2CCC(n3nc(-c4ccc(Oc5ccccc5)cc4)c4c(N)ncnc43)CC2)C([2H])([2H])C([2H])([2H])N(c2cc(F)c3c(c2)C(=O)N(C2CCC(=O)NC2=O)C3=O)C1([2H])[2H]] | 6-(4-((4-(4-amino-3-(4-phenoxyphenyl)-1H- pyrazolo[3,4-d]pyrimidin-1- yl)cyclohexyl)methyl)piperazin-1-yl- 2,2,3,3,5,5,6,6-d₈)-2-(2,6-dioxopiperidin-3-yl)- 4-fluoroisoindoline-1,3-dione |
| | 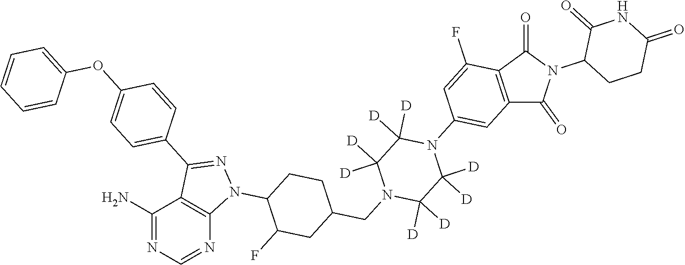 [SMILES: [2H]C1([2H])N(CC2CCC(n3nc(-c4ccc(Oc5ccccc5)cc4)c4c(N)ncnc43)C(F)C2)C([2H])([2H])C([2H])([2H])N(c2cc(F)c3c(c2)C(=O)N(C2CCC(=O)NC2=O)C3=O)C1([2H])[2H]] | 6-(4-((4-(4-amino-3-(4-phenoxyphenyl)-1H- pyrazolo[3,4-d]pyrimidin-1-yl)-3- fluorocyclohexyl)methyl)piperazin-1-yl- 2,2,3,3,5,5,6,6-d₈)-2-(2,6-dioxopiperidin-3-yl)- 4-fluoroisoindoline-1,3-dione |
| | 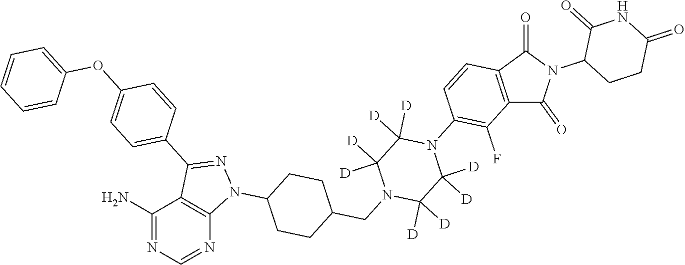 [SMILES: [2H]C1([2H])N(CC2CCC(n3nc(-c4ccc(Oc5ccccc5)cc4)c4c(N)ncnc43)CC2)C([2H])([2H])C([2H])([2H])N(c2ccc3c(c2F)C(=O)N(C2CCC(=O)NC2=O)C3=O)C1([2H])[2H]] | 5-(4-((4-(4-amino-3-(4-phenoxyphenyl)-1H- pyrazolo[3,4-d]pyrimidin-1- yl)cyclohexyl)methyl)piperazin-1-yl- 2,2,3,3,5,5,6,6-d₈)-2-(2,6-dioxopiperidin-3-yl)- 4-fluoroisoindoline-1,3-dione |
| | 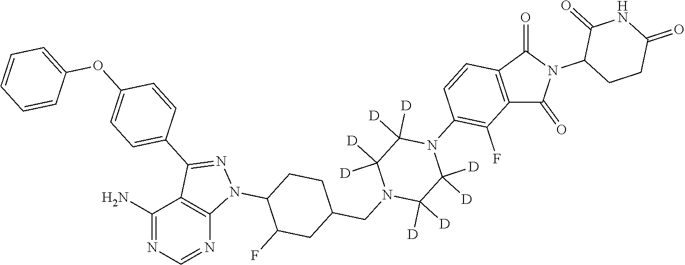 [SMILES: [2H]C1([2H])N(CC2CCC(n3nc(-c4ccc(Oc5ccccc5)cc4)c4c(N)ncnc43)C(F)C2)C([2H])([2H])C([2H])([2H])N(c2ccc3c(c2F)C(=O)N(C2CCC(=O)NC2=O)C3=O)C1([2H])[2H]] | 5-(4-((4-(4-amino-3-(4-phenoxyphenyl)-1H- pyrazolo[3,4-d]pyrimidin-1-yl)-3- fluorocyclohexyl)methyl)piperazin-1-yl- 2,2,3,3,5,5,6,6-d₈)-2-(2,6-dioxopiperidin-3-yl)- 4-fluoroisoindoline-1,3-dione |
| | 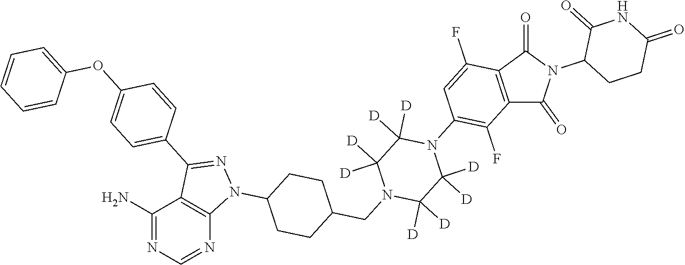 [SMILES: [2H]C1([2H])N(CC2CCC(n3nc(-c4ccc(Oc5ccccc5)cc4)c4c(N)ncnc43)CC2)C([2H])([2H])C([2H])([2H])N(c2cc(F)c3c(c2F)C(=O)N(C2CCC(=O)NC2=O)C3=O)C1([2H])[2H]] | 5-(4-((4-(4-amino-3-(4-phenoxyphenyl)-1H- pyrazolo[3,4-d]pyrimidin-1- yl)cyclohexyl)methyl)piperazin-1-yl- 2,2,3,3,5,5,6,6-d₈)-2-(2,6-dioxopiperidin-3-yl)- 4,7-difluoroisoindoline-1,3-dione |
| | 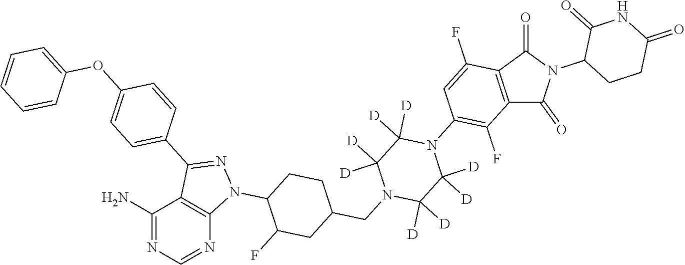 [SMILES: [2H]C1([2H])N(CC2CCC(n3nc(-c4ccc(Oc5ccccc5)cc4)c4c(N)ncnc43)C(F)C2)C([2H])([2H])C([2H])([2H])N(c2cc(F)c3c(c2F)C(=O)N(C2CCC(=O)NC2=O)C3=O)C1([2H])[2H]] | 5-(4-((4-(4-amino-3-(4-phenoxyphenyl)-1H- pyrazolo[3,4-d]pyrimidin-1-yl)-3- fluorocyclohexyl)methyl)piperazin-1-yl- 2,2,3,3,5,5,6,6-d₈)-2-(2,6-dioxopiperidin-3-yl)- 4,7-difluoroisoindoline-1,3-dione |
| | 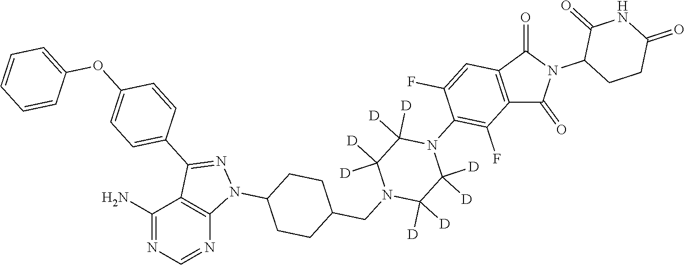 [SMILES: [2H]C1([2H])N(CC2CCC(n3nc(-c4ccc(Oc5ccccc5)cc4)c4c(N)ncnc43)CC2)C([2H])([2H])C([2H])([2H])N(c2c(F)cc3c(c2F)C(=O)N(C2CCC(=O)NC2=O)C3=O)C1([2H])[2H]] | 5-(4-((4-(4-amino-3-(4-phenoxyphenyl)-1H- pyrazolo[3,4-d]pyrimidin-1- yl)cyclohexyl)methyl)piperazin-1-yl- 2,2,3,3,5,5,6,6-d₈)-2-(2,6-dioxopiperidin-3-yl)- 4,6-difluoroisoindoline-1,3-dione |
| | 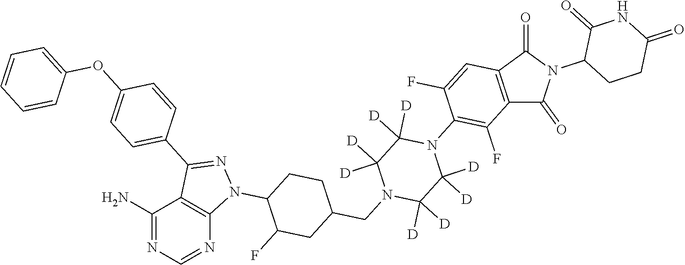 [SMILES: [2H]C1([2H])N(CC2CCC(n3nc(-c4ccc(Oc5ccccc5)cc4)c4c(N)ncnc43)C(F)C2)C([2H])([2H])C([2H])([2H])N(c2c(F)cc3c(c2F)C(=O)N(C2CCC(=O)NC2=O)C3=O)C1([2H])[2H]] | 5-(4-((4-(4-amino-3-(4-phenoxyphenyl)-1H- pyrazolo[3,4-d]pyrimidin-1-yl)-3- fluorocyclohexyl)methyl)piperazin-1-yl- 2,2,3,3,5,5,6,6-d₈)-2-(2,6-dioxopiperidin-3-yl)- 4,6-difluoroisoindoline-1,3-dione |
| | 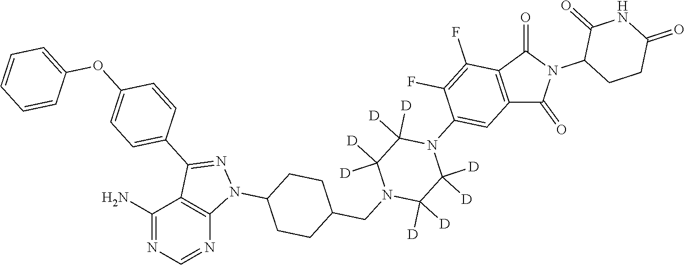 [SMILES: [2H]C1([2H])N(CC2CCC(n3nc(-c4ccc(Oc5ccccc5)cc4)c4c(N)ncnc43)CC2)C([2H])([2H])C([2H])([2H])N(c2cc3c(c(F)c2F)C(=O)N(C2CCC(=O)NC2=O)C3=O)C1([2H])[2H]] | 6-(4-((4-(4-amino-3-(4-phenoxyphenyl)-1H- pyrazolo[3,4-d]pyrimidin-1- yl)cyclohexyl)methyl)piperazin-1-yl- 2,2,3,3,5,5,6,6-d₈)-2-(2,6-dioxopiperidin-3-yl)- 4,5-difluoroisoindoline-1,3-dione |
| | 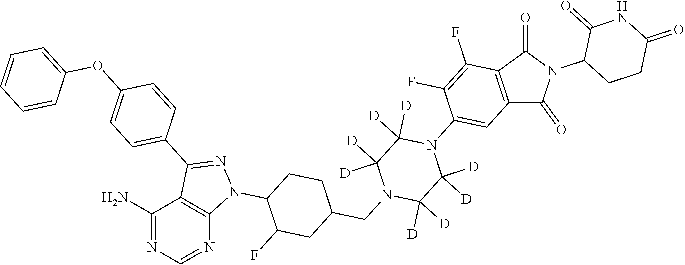 [SMILES: [2H]C1([2H])N(CC2CCC(n3nc(-c4ccc(Oc5ccccc5)cc4)c4c(N)ncnc43)C(F)C2)C([2H])([2H])C([2H])([2H])N(c2cc3c(c(F)c2F)C(=O)N(C2CCC(=O)NC2=O)C3=O)C1([2H])[2H]] | 6-(4-((4-(4-amino-3-(4-phenoxyphenyl)-1H- pyrazolo[3,4-d]pyrimidin-1-yl)-3- fluorocyclohexyl)methyl)piperazin-1-yl- 2,2,3,3,5,5,6,6-d₈)-2-(2,6-dioxopiperidin-3-yl)- 4,5-difluoroisoindoline-1,3-dione |
| | 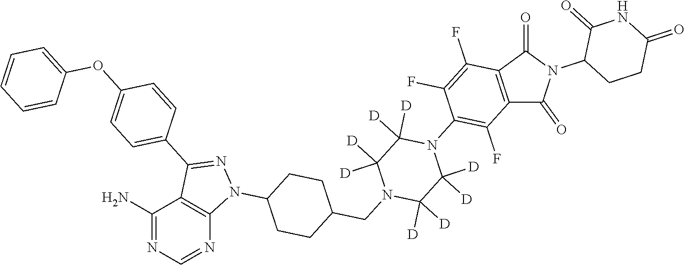 [SMILES: [2H]C1([2H])N(CC2CCC(n3nc(-c4ccc(Oc5ccccc5)cc4)c4c(N)ncnc43)CC2)C([2H])([2H])C([2H])([2H])N(c2c(F)c(F)c3c(c2F)C(=O)N(C2CCC(=O)NC2=O)C3=O)C1([2H])[2H]] | 5-(4-((4-(4-amino-3-(4-phenoxyphenyl)-1H- pyrazolo[3,4-d]pyrimidin-1- yl)cyclohexyl)methyl)piperazin-1-yl- 2,2,3,3,5,5,6,6-d₈)-2-(2,6-dioxopiperidin-3-yl)- 4,6,7-trifluoroisoindoline-1,3-dione |
| | 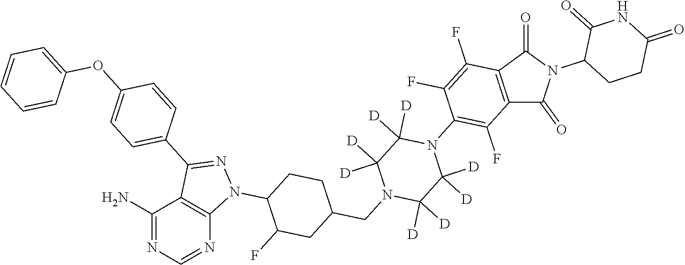 [SMILES: [2H]C1([2H])N(CC2CCC(n3nc(-c4ccc(Oc5ccccc5)cc4)c4c(N)ncnc43)C(F)C2)C([2H])([2H])C([2H])([2H])N(c2c(F)c(F)c3c(c2F)C(=O)N(C2CCC(=O)NC2=O)C3=O)C1([2H])[2H]] | 5-(4-((4-(4-amino-3-(4-phenoxyphenyl)-1H- pyrazolo[3,4-d]pyrimidin-1-yl)-3- fluorocyclohexyl)methyl)piperazin-1-yl- 2,2,3,3,5,5,6,6-d₈)-2-(2,6-dioxopiperidin-3-yl)- 4,6,7-trifluoroisoindoline-1,3-dione |
| | 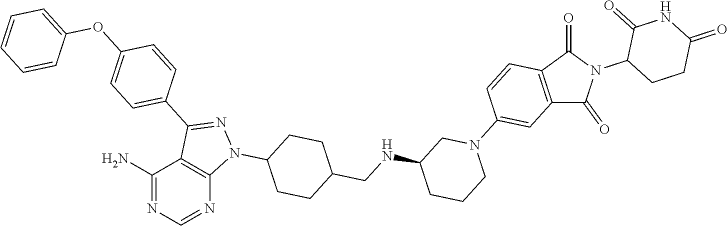 [SMILES: Nc1ncnc2c1c(-c1ccc(Oc3ccccc3)cc1)nn2C1CCC(CN[C@@H]2CCCN(c3ccc4c(c3)C(=O)N(C3CCC(=O)NC3=O)C4=O)C2)CC1] | 5-((R)-3-(((4-(4-amino-3-(4-phenoxyphenyl)- 1H-pyrazolo[3,4-d]pyrimidin-1- yl)cyclohexyl)methyl)amino)piperidin-1-yl)-2- (2,6-dioxopiperidin-3-yl)isoindoline-1,3-dione |
| | 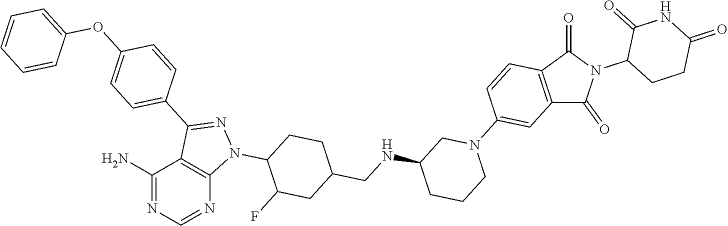 [SMILES: Nc1ncnc2c1c(-c1ccc(Oc3ccccc3)cc1)nn2C1CCC(CN[C@@H]2CCCN(c3ccc4c(c3)C(=O)N(C3CCC(=O)NC3=O)C4=O)C2)CC1F] | 5-((3R)-3-(((4-(4-amino-3-(4-phenoxyphenyl)- 1H-pyrazolo[3,4-d]pyrimidin-1-yl)-3- fluorocyclohexyl)methyl)amino)piperidin-1- yl)-2-(2,6-dioxopiperidin-3-yl)isoindoline-1,3- dione |
| | 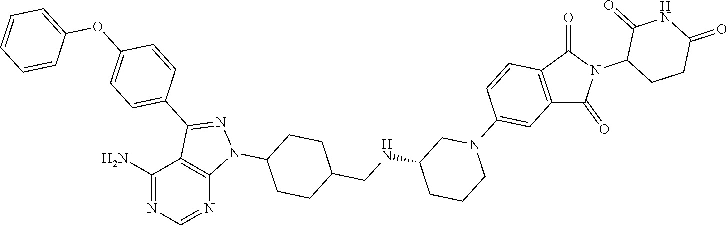 [SMILES: Nc1ncnc2c1c(-c1ccc(Oc3ccccc3)cc1)nn2C1CCC(CN[C@H]2CCCN(c3ccc4c(c3)C(=O)N(C3CCC(=O)NC3=O)C4=O)C2)CC1] | 5-((S)-3-(((4-(4-amino-3-(4-phenoxyphenyl)- 1H-pyrazolo[3,4-d]pyrimidin-1- yl)cyclohexyl)methyl)amino)piperidin-1-yl)-2- (2,6-dioxopiperidin-3-yl)isoindoline-1,3-dione |
| | 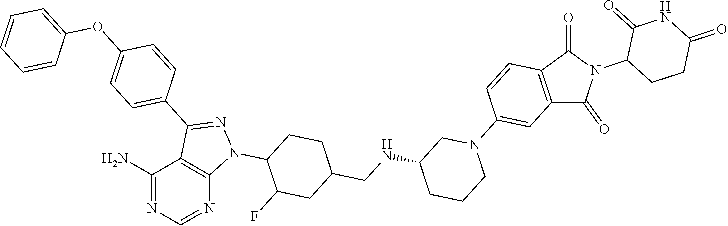 [SMILES: Nc1ncnc2c1c(-c1ccc(Oc3ccccc3)cc1)nn2C1CCC(CN[C@H]2CCCN(c3ccc4c(c3)C(=O)N(C3CCC(=O)NC3=O)C4=O)C2)CC1F] | 5-((3S)-3-(((4-(4-amino-3-(4-phenoxyphenyl)- 1H-pyrazolo[3,4-d]pyrimidin-1-yl)-3- fluorocyclohexyl)methyl)amino)piperidin-1- yl)-2-(2,6-dioxopiperidin-3-yl)isoindoline-1,3- dione |
| | 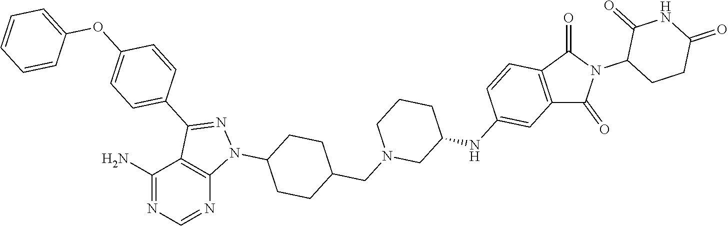 [SMILES: Nc1ncnc2c1c(-c1ccc(Oc3ccccc3)cc1)nn2C1CCC(CN2CCC[C@H](Nc3ccc4c(c3)C(=O)N(C3CCC(=O)NC3=O)C4=O)C2)CC1] | 5-(((S)-1-((4-(4-amino-3-(4-phenoxyphenyl)- 1H-pyrazolo[3,4-d]pyrimidin-1- yl)cyclohexyl)methyl)piperidin-3-yl)amino)-2- (2,6-dioxopiperidin-3-yl)isoindoline-1,3-dione |
| | 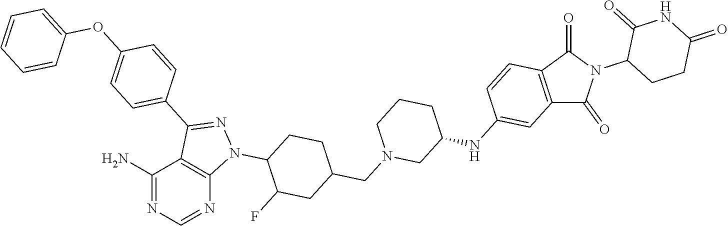 [SMILES: Nc1ncnc2c1c(-c1ccc(Oc3ccccc3)cc1)nn2C1CCC(CN2CCC[C@H](Nc3ccc4c(c3)C(=O)N(C3CCC(=O)NC3=O)C4=O)C2)CC1F] | 5-(((3S)-1 -((4-(4-amino-3-(4-phenoxyphenyl)- 1H-pyrazolo[3,4-d]pyrimidin-1-yl)-3- fluorocyclohexyl)methyl)piperidin-3- yl)amino)-2-(2,6-dioxopiperidin-3- yl)isoindoline-1,3-dione |
| | 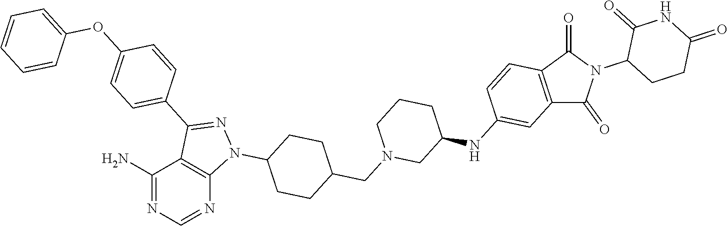 [SMILES: Nc1ncnc2c1c(-c1ccc(Oc3ccccc3)cc1)nn2C1CCC(CN2CCC[C@@H](Nc3ccc4c(c3)C(=O)N(C3CCC(=O)NC3=O)C4=O)C2)CC1] | 5-(((R)-1-((4-(4-amino-3-(4-phenoxyphenyl)- 1H-pyrazolo[3,4-d]pyrimidin-1- yl)cyclohexyl)methyl)piperidin-3-yl)amino)-2- (2,6-dioxopiperidin-3-yl)isoindoline-1,3-dione |
| | 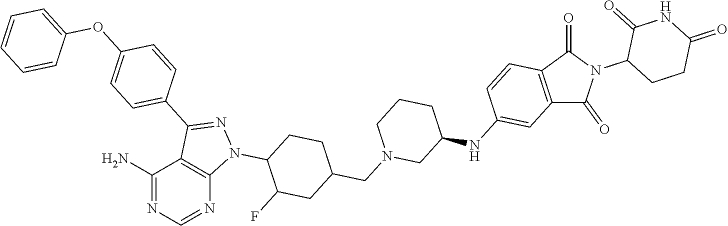 [SMILES: Nc1ncnc2c1c(-c1ccc(Oc3ccccc3)cc1)nn2C1CCC(CN2CCC[C@@H](Nc3ccc4c(c3)C(=O)N(C3CCC(=O)NC3=O)C4=O)C2)CC1F] | 5-(((3R)-1-((4-(4-amino-3-(4-phenoxyphenyl)- 1H-pyrazolo[3,4-d]pyrimidin-1-yl)-3- fluorocyclohexyl)methyl)piperidin-3- yl)amino)-2-(2,6-dioxopiperidin-3- yl)isoindoline-1,3-dione |
| | 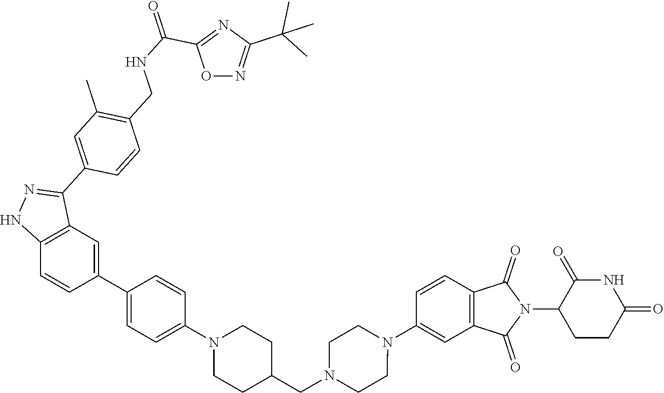 [SMILES: Cc1cc(-c2n[nH]c3ccc(-c4ccc(N5CCC(CN6CCN(c7ccc8c(c7)C(=O)N(C7CCC(=O)NC7=O)C8=O)CC6)CC5)cc4)cc23)ccc1CNC(=O)c1nc(C(C)(C)C)no1] | 3-(tert-butyl)-N-(4-(5-(4-(4-((4-(2-(2,6- dioxopiperidin-3-yl)-1,3-dioxoisoindolin-5- yl)piperazin-1-yl)methyl)piperidin-1- yl)phenyl)-1H-indazol-3-yl)-2-methylbenzyl)- 1,2,4-oxadiazole-5-carboxamide |
| | 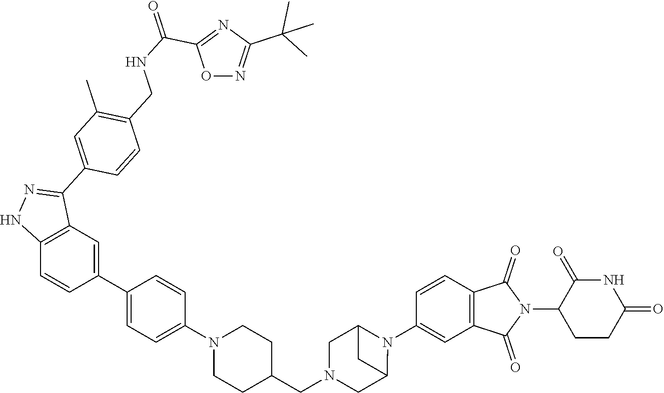 [SMILES: Cc1cc(-c2n[nH]c3ccc(-c4ccc(N5CCC(CN6CC7CC(C6)N7c6ccc7c(c6)C(=O)N(C6CCC(=O)NC6=O)C7=O)CC5)cc4)cc23)ccc1CNC(=O)c1nc(C(C)(C)C)no1] | 3-(tert-butyl)-N-(4-(5-(4-(4-((6-(2-(2,6- dioxopiperidin-3-yl)-1,3-dioxoisoindolin-5-yl)- 3,6-diazabicyclo[3.1.1]heptan-3- yl)methyl)piperidin-1-yl)phenyl)-1H-indazol-3- yl)-2-methylbenzyl)-1,2,4-oxadiazole-5- carboxamide |
| | 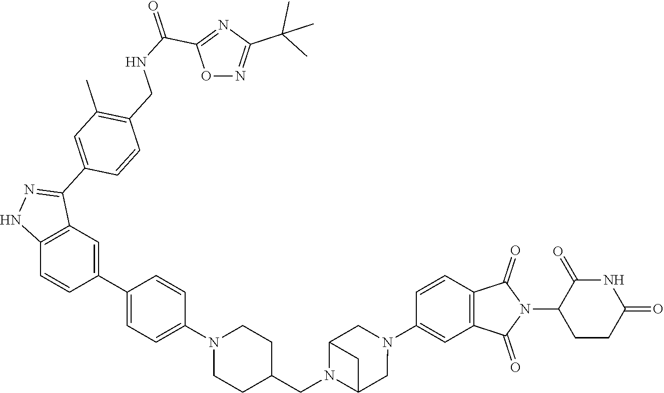 [SMILES: Cc1cc(-c2n[nH]c3ccc(-c4ccc(N5CCC(CN6C7CC6CN(c6ccc8c(c6)C(=O)N(C6CCC(=O)NC6=O)C8=O)C7)CC5)cc4)cc23)ccc1CNC(=O)c1nc(C(C)(C)C)no1] | 3-(tert-butyl)-N-(4-(5-(4-(4-((3-(2-(2,6- dioxopiperidin-3-yl)-1,3-dioxoisoindolin-5-yl)- 3,6-diazabicyclo[3.1.1]heptan-6- yl)methyl)piperidin-1-yl)phenyl)-1H-indazol-3- yl)-2-methylbenzyl)-1,2,4-oxadiazole-5- carboxamide |
| | 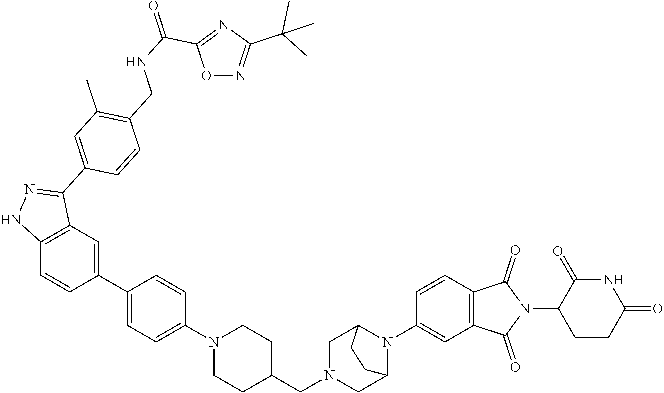 [SMILES: Cc1cc(-c2n[nH]c3ccc(-c4ccc(N5CCC(CN6CC7CCC(C6)N7c6ccc7c(c6)C(=O)N(C6CCC(=O)NC6=O)C7=O)CC5)cc4)cc23)ccc1CNC(=O)c1nc(C(C)(C)C)no1] | 3-(tert-butyl)-N-(4-(5-(4-(4-((8-(2-(2,6- dioxopiperidin-3-yl)-1,3-dioxoisoindolin-5-yl)- 3,8-diazabicyclo[3.2.1]octan-3- yl)methyl)piperidin-1-yl)phenyl)-1H-indazol-3- yl)-2-methylbenzyl)-1,2,4-oxadiazole-5- carboxamide |
| | 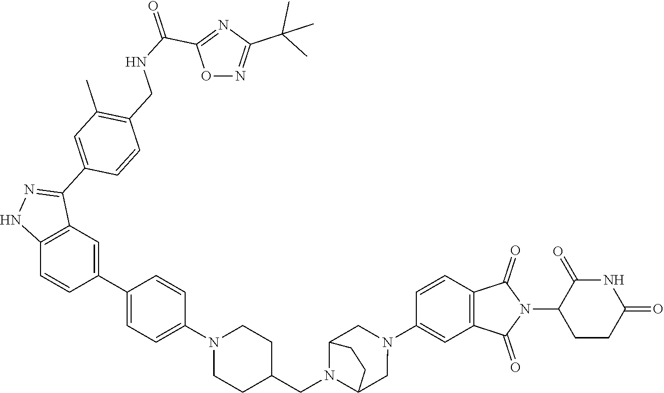 [SMILES: Cc1cc(-c2n[nH]c3ccc(-c4ccc(N5CCC(CN6C7CCC6CN(c6ccc8c(c6)C(=O)N(C6CCC(=O)NC6=O)C8=O)C7)CC5)cc4)cc23)ccc1CNC(=O)c1nc(C(C)(C)C)no1] | 3-(tert-butyl)-N-(4-(5-(4-(4-((3-(2-(2,6- dioxopiperidin-3-yl)-1,3-dioxoisoindolin-5-yl)- 3,8-diazabicyclo[3.2.1]octan-8- yl)methyl)piperidin-1-yl)phenyl)-1H-indazol-3- yl)-2-methylbenzyl)-1,2,4-oxadiazole-5- carboxamide |
| | 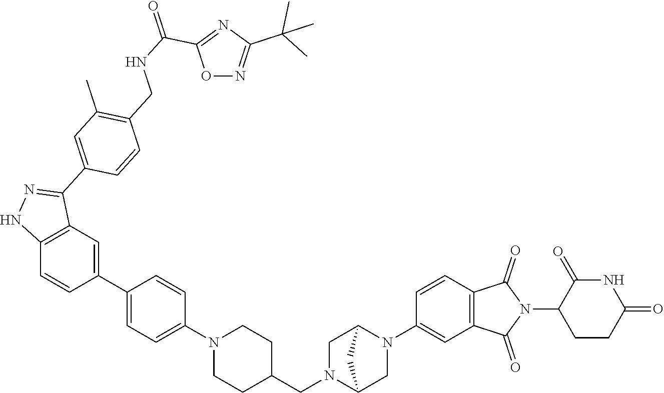 [SMILES: Cc1cc(-c2n[nH]c3ccc(-c4ccc(N5CCC(CN6C[C@@H]7C[C@H]6CN7c6ccc7c(c6)C(=O)N(C6CCC(=O)NC6=O)C7=O)CC5)cc4)cc23)ccc1CNC(=O)c1nc(C(C)(C)C)no1] | 3-(tert-butyl)-N-(4-(5-(4-(4-(((1S,4S)-5-(2-(2,6- dioxopiperidin-3-yl)-1,3-dioxoisoindolin-5-yl)- 2,5-diazabicyclo[2.2.1]heptan-2- yl)methyl)piperidin-1-yl)phenyl)-1H-indazol-3- yl)-2-methylbenzyl)-1,2,4-oxadiazole-5- carboxamide |
| | 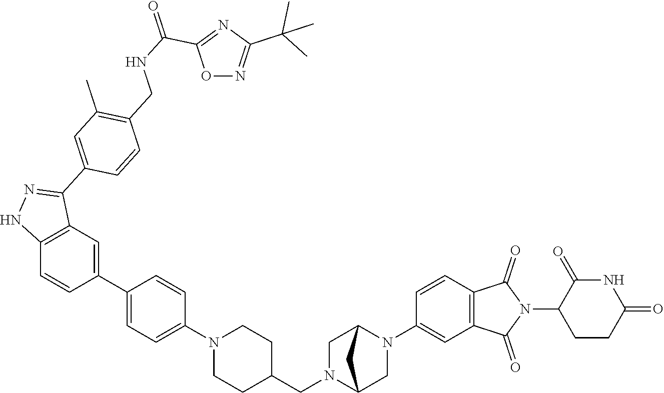 [SMILES: Cc1cc(-c2n[nH]c3ccc(-c4ccc(N5CCC(CN6C[C@H]7C[C@@H]6CN7c6ccc7c(c6)C(=O)N(C6CCC(=O)NC6=O)C7=O)CC5)cc4)cc23)ccc1CNC(=O)c1nc(C(C)(C)C)no1] | 3-(tert-butyl)-N-(4-(5-(4-(4-(((1R,4R)-5-(2- (2,6-dioxopiperidin-3-yl)-1,3-dioxoisoindolin- 5-yl)-2,5-diazabicyclo[2.2.1]heptan-2- yl)methyl)piperidin-1-yl)phenyl)-1H-indazol-3- yl)-2-methylbenzyl)-1,2,4-oxadiazole-5- carboxamide |
| | 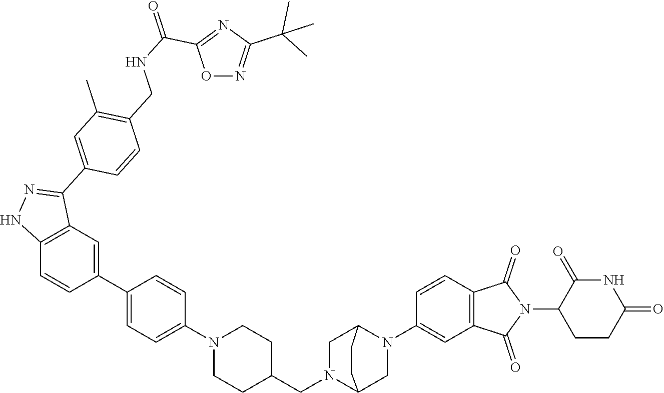 [SMILES: Cc1cc(-c2n[nH]c3ccc(-c4ccc(N5CCC(CN6CC7CCC6CN7c6ccc7c(c6)C(=O)N(C6CCC(=O)NC6=O)C7=O)CC5)cc4)cc23)ccc1CNC(=O)c1nc(C(C)(C)C)no1] | 3-(tert-butyl)-N-(4-(5-(4-(4-((5-(2-(2,6- dioxopiperidin-3-yl)-1,3-dioxoisoindolin-5-yl)- 2,5-diazabicyclo[2.2.2]octan-2- yl)methyl)piperidin-1-yl)phenyl)-1H-indazol-3- yl)-2-methylbenzyl)-1,2,4-oxadiazole-5- carboxamide |
| | 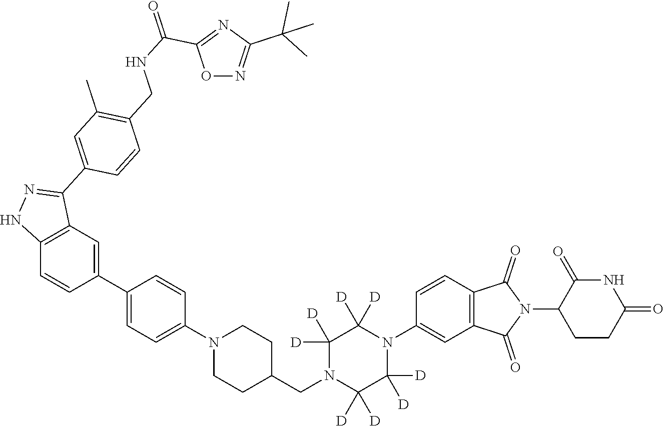 [SMILES: [2H]C1([2H])N(CC2CCN(c3ccc(-c4ccc5[nH]nc(-c6ccc(CNC(=O)c7nc(C(C)(C)C)no7)c(C)c6)c5c4)cc3)CC2)C([2H])([2H])C([2H])([2H])N(c2ccc3c(c2)C(=O)N(C2CCC(=O)NC2=O)C3=O)C1([2H])[2H]] | 3-(tert-butyl)-N-(4-(5-(4-(4-((4-(2-(2,6- dioxopiperidin-3-yl)-1,3-dioxoisoindolin-5- yl)piperazin-1-yl-2,2,3,3,5,5,6,6- d₈)methyl)piperidin-1-yl)phenyl)-1H-indazol-3- yl)-2-methylbenzyl)-1,2,4-oxadiazole-5- carboxamide |
| | 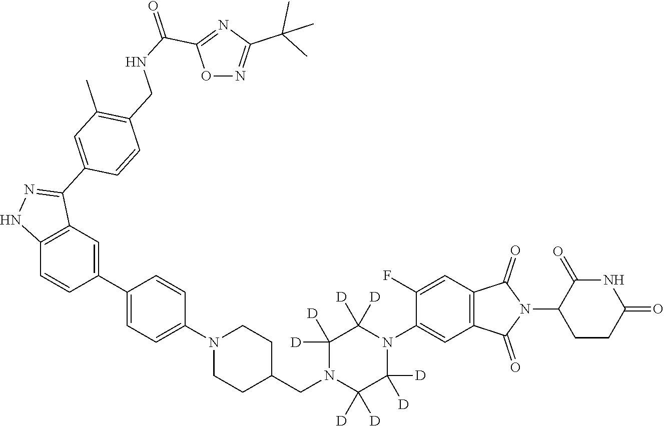 [SMILES: [2H]C1([2H])N(CC2CCN(c3ccc(-c4ccc5[nH]nc(-c6ccc(CNC(=O)c7nc(C(C)(C)C)no7)c(C)c6)c5c4)cc3)CC2)C([2H])([2H])C([2H])([2H])N(c2cc3c(cc2F)C(=O)N(C2CCC(=O)NC2=O)C3=O)C1([2H])[2H]] | 3-(tert-butyl)-N-(4-(5-(4-(4-((4-(2-(2,6- dioxopiperidin-3-yl)-6-fluoro-1,3- dioxoisoindolin-5-yl)piperazin-1-yl- 2,2,3,3,5,5,6,6-d₈)methyl)piperidin-1- yl)phenyl)-1H-indazol-3-yl)-2-methylbenzyl)- 1,2,4-oxadiazole-5-carboxamide |
| | 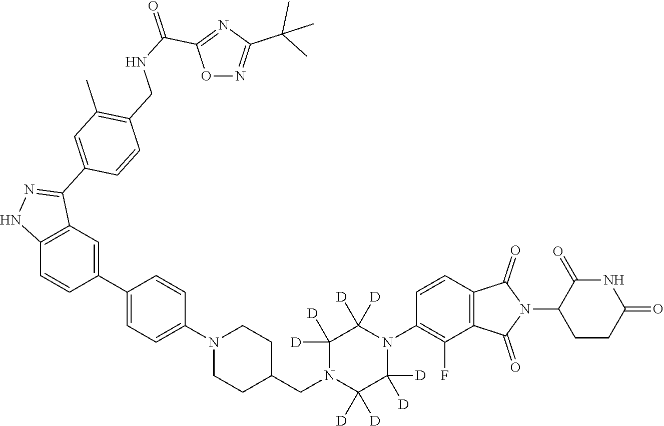 [SMILES: [2H]C1([2H])N(CC2CCN(c3ccc(-c4ccc5[nH]nc(-c6ccc(CNC(=O)c7nc(C(C)(C)C)no7)c(C)c6)c5c4)cc3)CC2)C([2H])([2H])C([2H])([2H])N(c2ccc3c(c2F)C(=O)N(C2CCC(=O)NC2=O)C3=O)C1([2H])[2H]] | 3-(tert-butyl)-N-(4-(5-(4-(4-((4-(2-(2,6- dioxopiperidin-3-yl)-4-fluoro-1,3- dioxoisoindolin-5-yl)piperazin-1-yl- 2,2,3,3,5,5,6,6-d₈)methyl)piperidin-1- yl)phenyl)-1H-indazol-3-yl)-2-methylbenzyl)- 1,2,4-oxadiazole-5-carboxamide |
| | 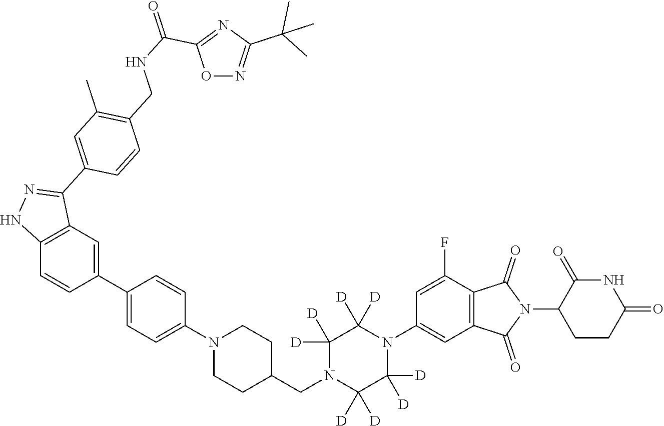 [SMILES: [2H]C1([2H])N(CC2CCN(c3ccc(-c4ccc5[nH]nc(-c6ccc(CNC(=O)c7nc(C(C)(C)C)no7)c(C)c6)c5c4)cc3)CC2)C([2H])([2H])C([2H])([2H])N(c2cc(F)c3c(c2)C(=O)N(C2CCC(=O)NC2=O)C3=O)C1([2H])[2H]] | 3-(tert-butyl)-N-(4-(5-(4-(4-((4-(2-(2,6- dioxopiperidin-3-yl)-7-fluoro-1,3- dioxoisoindolin-5-yl)piperazin-1-yl- 2,2,3,3,5,5,6,6-d₈)methyl)piperidin-1- yl)phenyl)-1H-indazol-3-yl)-2-methylbenzyl)- 1,2,4-oxadiazole-5-carboxamide |
| | 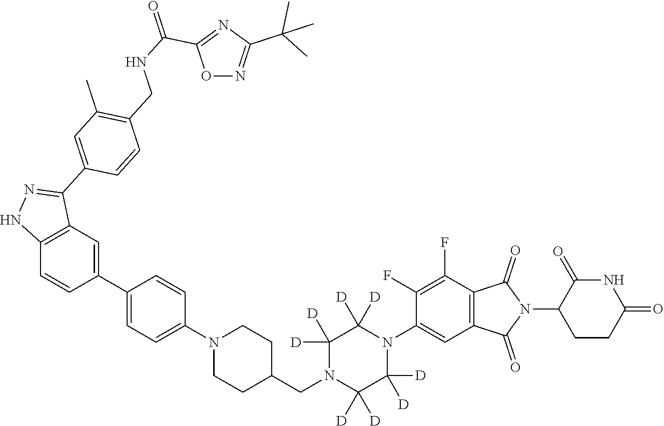 [SMILES: [2H]C1([2H])N(CC2CCN(c3ccc(-c4ccc5[nH]nc(-c6ccc(CNC(=O)c7nc(C(C)(C)C)no7)c(C)c6)c5c4)cc3)CC2)C([2H])([2H])C([2H])([2H])N(c2cc3c(c(F)c2F)C(=O)N(C2CCC(=O)NC2=O)C3=O)C1([2H])[2H]] | 3-(tert-butyl)-N-(4-(5-(4-(4-((4-(2-(2,6- dioxopiperidin-3-yl)-6,7-difluoro-1,3- dioxoisoindolin-5-yl)piperazin-1-yl- 2,2,3,3,5,5,6,6-d₈)methyl)piperidin-1- yl)phenyl)-1H-indazol-3-yl)-2-methylbenzyl)- 1,2,4-oxadiazole-5-carboxamide |
| | 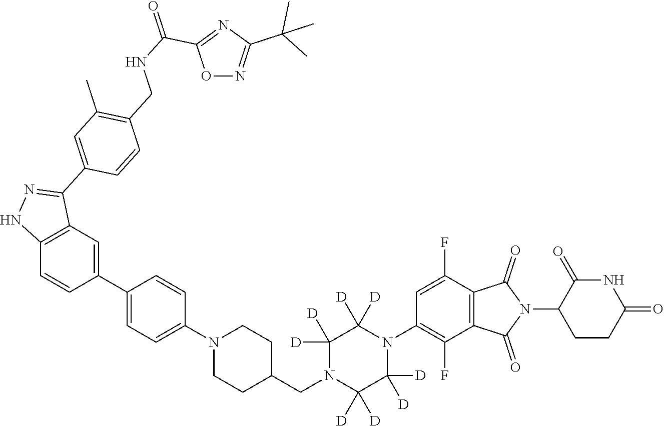 [SMILES: [2H]C1([2H])N(CC2CCN(c3ccc(-c4ccc5[nH]nc(-c6ccc(CNC(=O)c7nc(C(C)(C)C)no7)c(C)c6)c5c4)cc3)CC2)C([2H])([2H])C([2H])([2H])N(c2cc(F)c3c(c2F)C(=O)N(C2CCC(=O)NC2=O)C3=O)C1([2H])[2H]] | 3-(tert-butyl)-N-(4-(5-(4-(4-((4-(2-(2,6- dioxopiperidin-3-yl)-4,7-difluoro-1,3- dioxoisoindolin-5-yl)piperazin-1-yl- 2,2,3,3,5,5,6,6-d₈)methyl)piperidin-1- yl)phenyl)-1H-indazol-3-yl)-2-methylbenzyl)- 1,2,4-oxadiazole-5-carboxamide |
| | 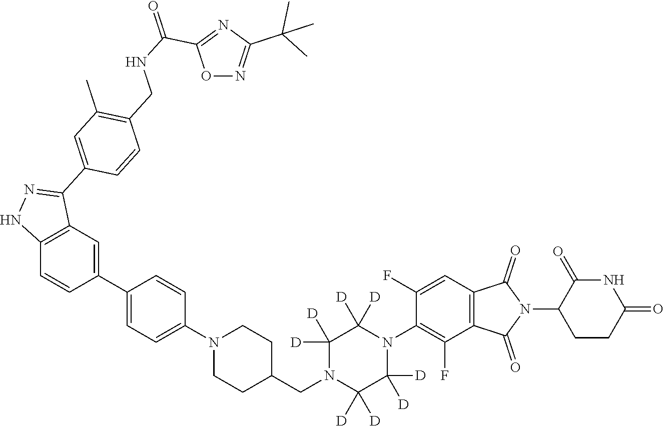 [SMILES: [2H]C1([2H])N(CC2CCN(c3ccc(-c4ccc5[nH]nc(-c6ccc(CNC(=O)c7nc(C(C)(C)C)no7)c(C)c6)c5c4)cc3)CC2)C([2H])([2H])C([2H])([2H])N(c2c(F)cc3c(c2F)C(=O)N(C2CCC(=O)NC2=O)C3=O)C1([2H])[2H]] | 3-(tert-butyl)-N-(4-(5-(4-(4-((4-(2-(2,6- dioxopiperidin-3-yl)-4,6-difluoro-1,3- dioxoisoindolin-5-yl)piperazin-1-yl- 2,2,3,3,5,5,6,6-d₈)methyl)piperidin-1- yl)phenyl)-1H-indazol-3-yl)-2-methylbenzyl)- 1,2,4-oxadiazole-5-carboxamide |
| | 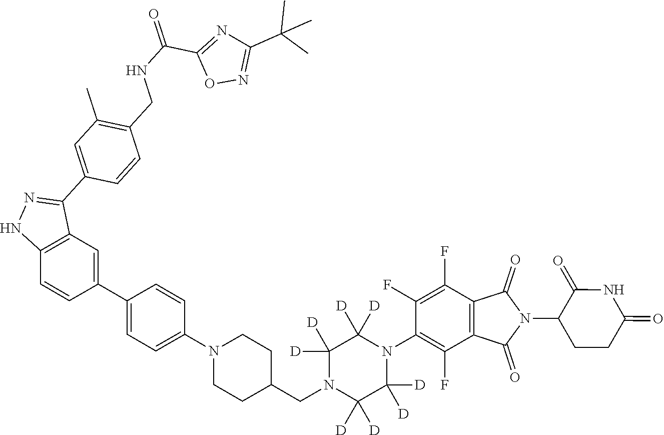 [SMILES: [2H]C1([2H])N(CC2CCN(c3ccc(-c4ccc5[nH]nc(-c6ccc(CNC(=O)c7nc(C(C)(C)C)no7)c(C)c6)c5c4)cc3)CC2)C([2H])([2H])C([2H])([2H])N(c2c(F)c(F)c3c(c2F)C(=O)N(C2CCC(=O)NC2=O)C3=O)C1([2H])[2H]] | 3-(tert-butyl)-N-(4-(5-(4-(4-((4-(2-(2,6- dioxopiperidin-3-yl)-4,6,7-trifluoro-1,3- dioxoisoindolin-5-yl)piperazin-1-yl- 2,2,3,3,5,5,6,6-d₈)methyl)piperidin-1- yl)phenyl)-1H-indazol-3-yl)-2-methylbenzyl)- 1,2,4-oxadiazole-5-carboxamide |
| | 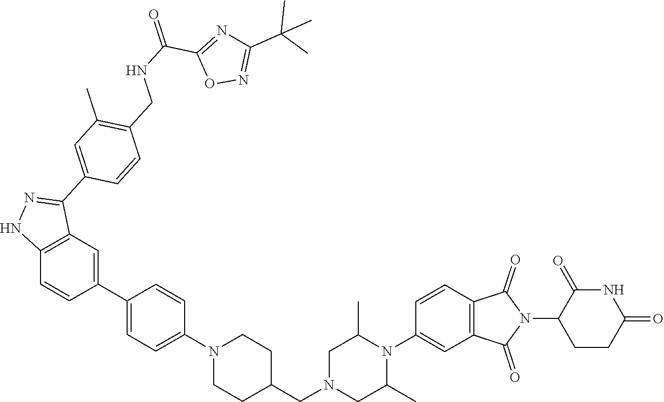 [SMILES: Cc1cc(-c2n[nH]c3ccc(-c4ccc(N5CCC(CN6CC(C)N(c7ccc8c(c7)C(=O)N(C7CCC(=O)NC7=O)C8=O)C(C)C6)CC5)cc4)cc23)ccc1CNC(=O)c1nc(C(C)(C)C)no1] | 3-(tert-butyl)-N-(4-(5-(4-(4-((4-(2-(2,6- dioxopiperidin-3-yl)-1,3-dioxoisoindolin-5-yl)- 3,5-dimethylpiperazin-1-yl)methyl)piperidin-1- yl)phenyl)-1H-indazol-3-yl)-2-methylbenzyl)- 1,2,4-oxadiazole-5-carboxamide |
| | 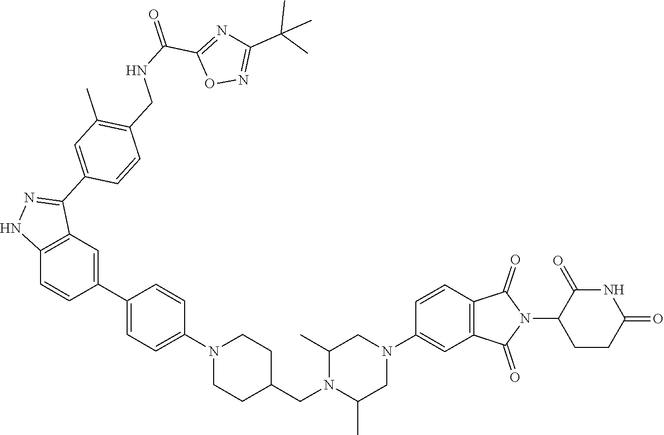 [SMILES: Cc1cc(-c2n[nH]c3ccc(-c4ccc(N5CCC(CN6C(C)CN(c7ccc8c(c7)C(=O)N(C7CCC(=O)NC7=O)C8=O)CC6C)CC5)cc4)cc23)ccc1CNC(=O)c1nc(C(C)(C)C)no1] | 3-(tert-butyl)-N-(4-(5-(4-(4-((4-(2-(2,6- dioxopiperidin-3-yl)-1,3-dioxoisoindolin-5-yl)- 2,6-dimethylpiperazin-1-yl)methyl)piperidin-1- yl)phenyl)-1H-indazol-3-yl)-2-methylbenzyl)- 1,2,4-oxadiazole-5-carboxamide |
| | 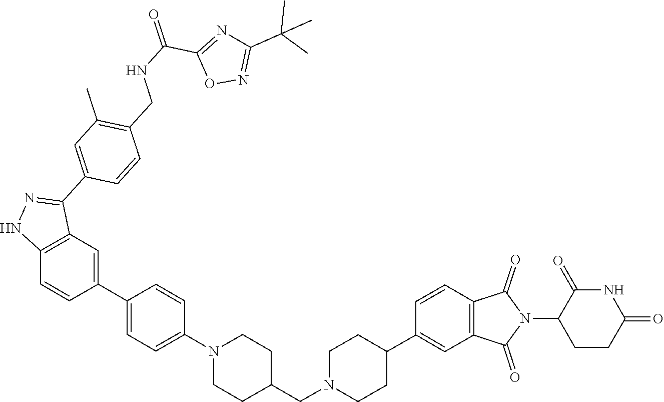 [SMILES: Cc1cc(-c2n[nH]c3ccc(-c4ccc(N5CCC(CN6CCC(c7ccc8c(c7)C(=O)N(C7CCC(=O)NC7=O)C8=O)CC6)CC5)cc4)cc23)ccc1CNC(=O)c1nc(C(C)(C)C)no1] | 3-(tert-butyl)-N-(4-(5-(4-(4-((4-(2-(2,6- dioxopiperidin-3-yl)-1,3-dioxoisoindolin-5- yl)piperidin-1-yl)methyl)piperidin-1- yl)phenyl)-1H-indazol-3-yl)-2-methylbenzyl)- 1,2,4-oxadiazole-5-carboxamide |
| | 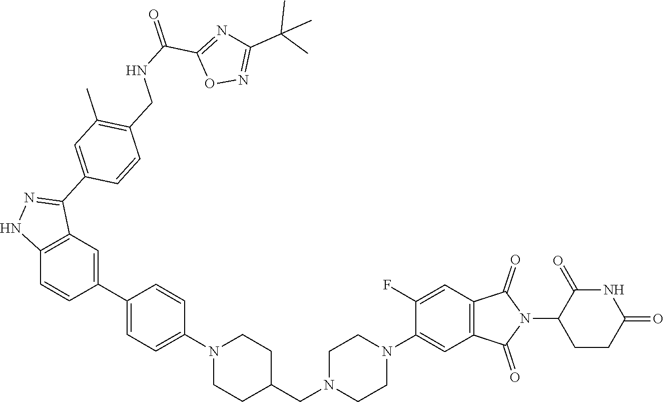 [SMILES: Cc1cc(-c2n[nH]c3ccc(-c4ccc(N5CCC(CN6CCN(c7cc8c(cc7F)C(=O)N(C7CCC(=O)NC7=O)C8=O)CC6)CC5)cc4)cc23)ccc1CNC(=O)c1nc(C(C)(C)C)no1] | 3-(tert-butyl)-N-(4-(5-(4-(4-((4-(2-(2,6- dioxopiperidin-3-yl)-6-fluoro-1,3- dioxoisoindolin-5-yl)piperazin-1- yl)methyl)piperidin-1-yl)phenyl)-1H-indazol-3- yl)-2-methylbenzyl)-1,2,4-oxadiazole-5- carboxamide |
| | 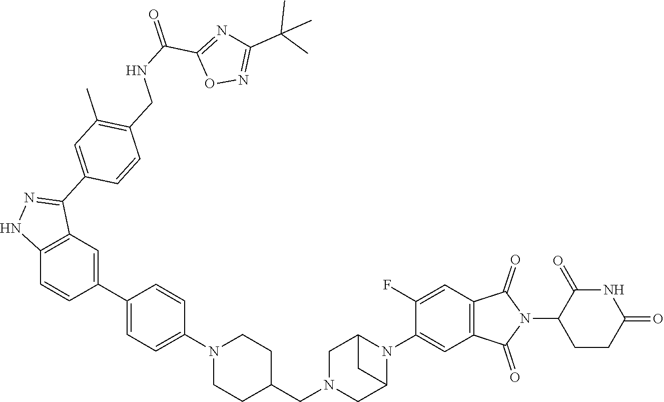 [SMILES: Cc1cc(-c2n[nH]c3ccc(-c4ccc(N5CCC(CN6CC7CC(C6)N7c6cc7c(cc6F)C(=O)N(C6CCC(=O)NC6=O)C7=O)CC5)cc4)cc23)ccc1CNC(=O)c1nc(C(C)(C)C)no1] | 3-(tert-butyl)-N-(4-(5-(4-(4-((6-(2-(2,6- dioxopiperidin-3-yl)-6-fluoro-1,3- dioxoisoindolin-5-yl)-3,6- diazabicyclo[3.1.1]heptan-3- yl)methyl)piperidin-1-yl)phenyl)-1H-indazol-3- yl)-2-methylbenzyl)-1,2,4-oxadiazole-5- carboxamide |
| | 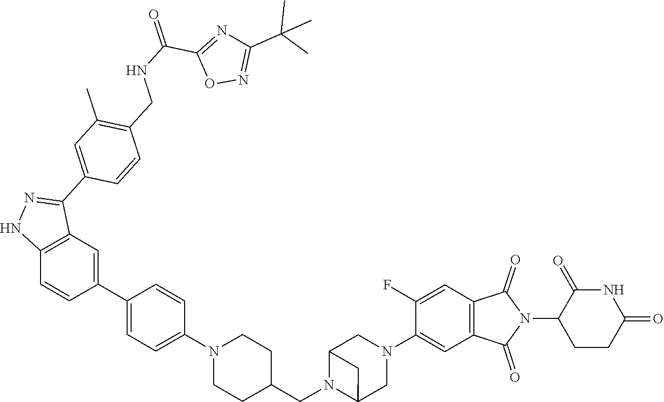 [SMILES: Cc1cc(-c2n[nH]c3ccc(-c4ccc(N5CCC(CN6C7CC6CN(c6cc8c(cc6F)C(=O)N(C6CCC(=O)NC6=O)C8=O)C7)CC5)cc4)cc23)ccc1CNC(=O)c1nc(C(C)(C)C)no1] | 3-(tert-butyl)-N-(4-(5-(4-(4-((3-(2-(2,6- dioxopiperidin-3-yl)-6-fluoro-1,3- dioxoisoindolin-5-yl)-3,6- diazabicyclo[3.1.1]heptan-6- yl)methyl)piperidin-1-yl)phenyl)-1H-indazol-3- yl)-2-methylbenzyl)-1,2,4-oxadiazole-5- carboxamide |
| | 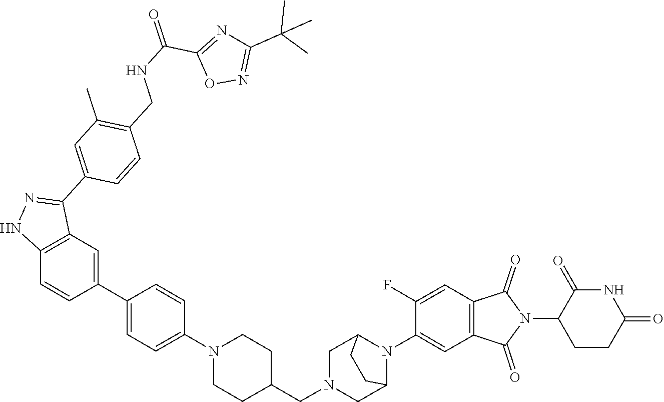 [SMILES: Cc1cc(-c2n[nH]c3ccc(-c4ccc(N5CCC(CN6CC7CCC(C6)N7c6cc7c(cc6F)C(=O)N(C6CCC(=O)NC6=O)C7=O)CC5)cc4)cc23)ccc1CNC(=O)c1nc(C(C)(C)C)no1] | 3-(tert-butyl)-N-(4-(5-(4-(4-((8-(2-(2,6- dioxopiperidin-3-yl)-6-fluoro-1,3- dioxoisoindolin-5-yl)-3,8- diazabicyclo[3.2.1]octan-3-yl)methyl)piperidin- 1-yl)phenyl)-1H-indazol-3-yl)-2- methylbenzyl)-1,2,4-oxadiazole-5-carboxamide |
| | 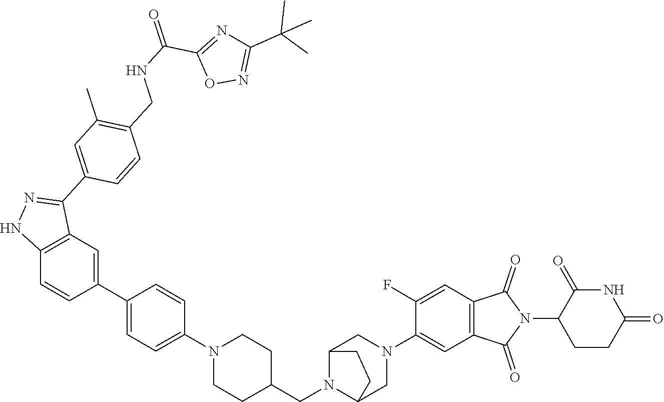 [SMILES: Cc1cc(-c2n[nH]c3ccc(-c4ccc(N5CCC(CN6C7CCC6CN(c6cc8c(cc6F)C(=O)N(C6CCC(=O)NC6=O)C8=O)C7)CC5)cc4)cc23)ccc1CNC(=O)c1nc(C(C)(C)C)no1] | 3-(tert-butyl)-N-(4-(5-(4-(4-((3-(2-(2,6- dioxopiperidin-3-yl)-6-fluoro-1,3- dioxoisoindolin-5-yl)-3,8- diazabicyclo[3.2.1]octan-8-yl)methyl)piperidin- 1-yl)phenyl)-1H-indazol-3-yl)-2- methylbenzyl)-1,2,4-oxadiazole-5-carboxamide |
| | 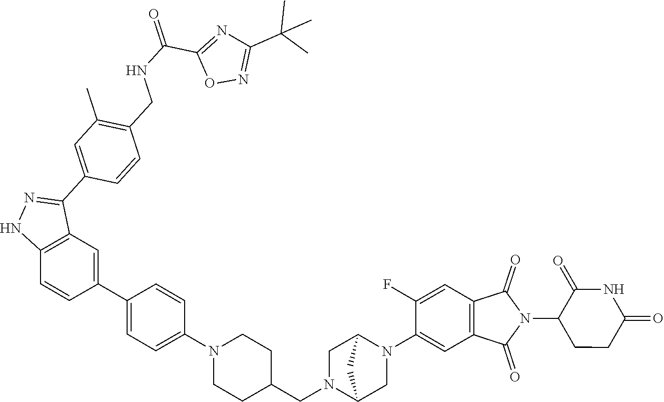 [SMILES: Cc1cc(-c2n[nH]c3ccc(-c4ccc(N5CCC(CN6C[C@@H]7C[C@H]6CN7c6cc7c(cc6F)C(=O)N(C6CCC(=O)NC6=O)C7=O)CC5)cc4)cc23)ccc1CNC(=O)c1nc(C(C)(C)C)no1] | 3-(tert-butyl)-N-(4-(5-(4-(4-(((1S,4S)-5-(2-(2,6- dioxopiperidin-3-yl)-6-fluoro-1,3- dioxoisoindolin-5-yl)-2,5- diazabicyclo[2.2.1]heptan-2- yl)methyl)piperidin-1-yl)phenyl)-1H-indazol-3- yl)-2-methylbenzyl)-1,2,4-oxadiazole-5- carboxamide |
| | 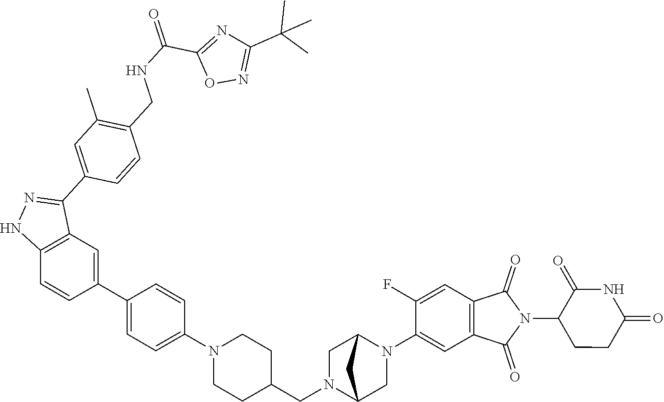 [SMILES: Cc1cc(-c2n[nH]c3ccc(-c4ccc(N5CCC(CN6C[C@H]7C[C@@H]6CN7c6cc7c(cc6F)C(=O)N(C6CCC(=O)NC6=O)C7=O)CC5)cc4)cc23)ccc1CNC(=O)c1nc(C(C)(C)C)no1] | 3-(tert-butyl)-N-(4-(5-(4-(4-(((1R,4R)-5-(2- (2,6-dioxopiperidin-3-yl)-6-fluoro-1,3- dioxoisoindolin-5-yl)-2,5- diazabicyclo[2.2.1]heptan-2- yl)methyl)piperidin-1-yl)phenyl)-1H-indazol-3- yl)-2-methylbenzyl)-1,2,4-oxadiazole-5- carboxamide |
| | 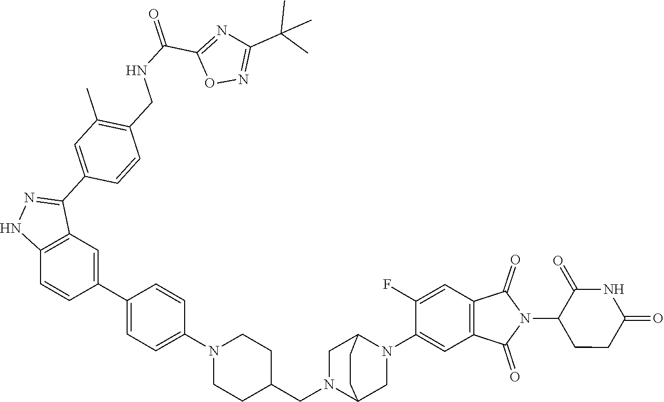 [SMILES: Cc1cc(-c2n[nH]c3ccc(-c4ccc(N5CCC(CN6CC7CCC6CN7c6cc7c(cc6F)C(=O)N(C6CCC(=O)NC6=O)C7=O)CC5)cc4)cc23)ccc1CNC(=O)c1nc(C(C)(C)C)no1] | 3-(tert-butyl)-N-(4-(5-(4-(4-((5-(2-(2,6- dioxopiperidin-3-yl)-6-fluoro-1,3- dioxoisoindolin-5-yl)-2,5- diazabicyclo[2.2.2]octan-2-yl)methyl)piperidin- 1-yl)phenyl)-1H-indazol-3-yl)-2- methylbenzyl)-1,2,4-oxadiazole-5-carboxamide |
| | 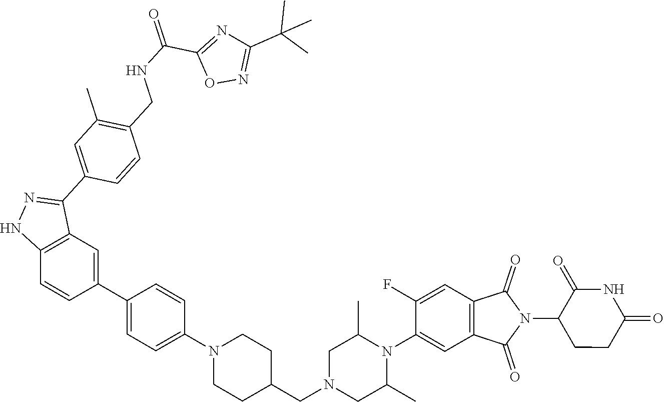 [SMILES: Cc1cc(-c2n[nH]c3ccc(-c4ccc(N5CCC(CN6CC(C)N(c7cc8c(cc7F)C(=O)N(C7CCC(=O)NC7=O)C8=O)C(C)C6)CC5)cc4)cc23)ccc1CNC(=O)c1nc(C(C)(C)C)no1] | 3-(tert-butyl)-N-(4-(5-(4-(4-((4-(2-(2,6- dioxopiperidin-3-yl)-6-fluoro-1,3- dioxoisoindolin-5-yl)-3,5-dimethylpiperazin-1 - yl)methyl)piperidin-1-yl)phenyl)-1H-indazol-3- yl)-2-methylbenzyl)-1,2,4-oxadiazole-5- carboxamide |
| | 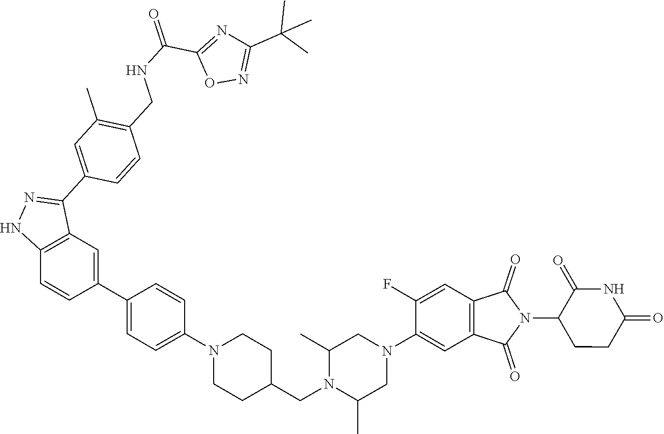 [SMILES: Cc1cc(-c2n[nH]c3ccc(-c4ccc(N5CCC(CN6C(C)CN(c7cc8c(cc7F)C(=O)N(C7CCC(=O)NC7=O)C8=O)CC6C)CC5)cc4)cc23)ccc1CNC(=O)c1nc(C(C)(C)C)no1] | 3-(tert-butyl)-N-(4-(5-(4-(4-((4-(2-(2,6- dioxopiperidin-3-yl)-6-fluoro-1,3- dioxoisoindolin-5-yl)-2,6-dimethylpiperazin-1- yl)methyl)piperidin-1-yl)phenyl)-1H-indazol-3- yl)-2-methylbenzyl)-1,2,4-oxadiazole-5- carboxamide |
| | 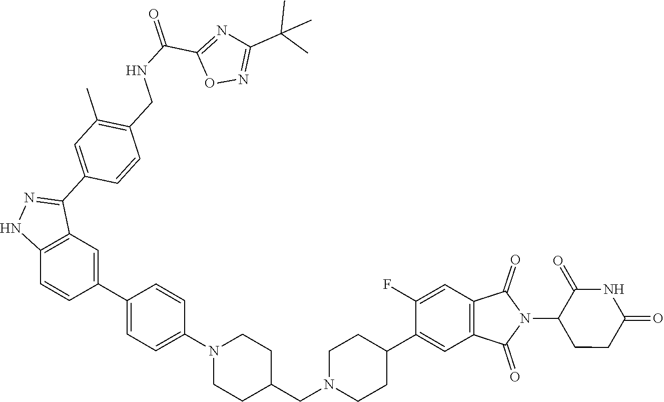 [SMILES: Cc1cc(-c2n[nH]c3ccc(-c4ccc(N5CCC(CN6CCC(c7cc8c(cc7F)C(=O)N(C7CCC(=O)NC7=O)C8=O)CC6)CC5)cc4)cc23)ccc1CNC(=O)c1nc(C(C)(C)C)no1] | 3-(tert-butyl)-N-(4-(5-(4-(4-((4-(2-(2,6- dioxopiperidin-3-yl)-6-fluoro-1,3- dioxoisoindolin-5-yl)piperidin-1- yl)methyl)piperidin-1-yl)phenyl)-1H-indazol-3- yl)-2-methylbenzyl)-1,2,4-oxadiazole-5- carboxamide |
| | 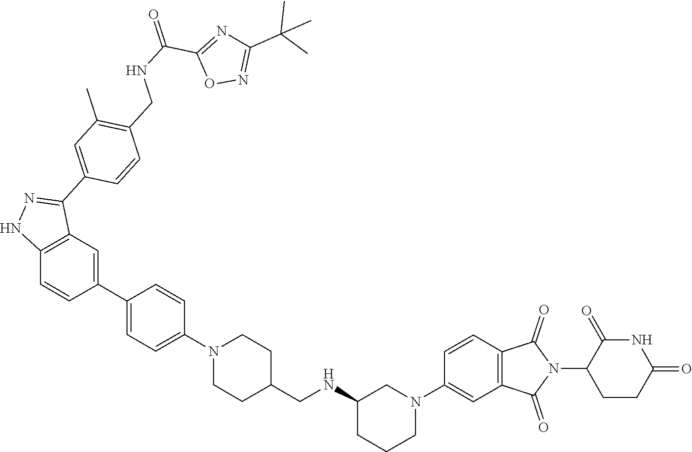 [SMILES: Cc1cc(-c2n[nH]c3ccc(-c4ccc(N5CCC(CN[C@@H]6CCCN(c7ccc8c(c7)C(=O)N(C7CCC(=O)NC7=O)C8=O)C6)CC5)cc4)cc23)ccc1CNC(=O)c1nc(C(C)(C)C)no1] | 3-(tert-butyl)-N-(4-(5-(4-(4-((((3R)-1-(2-(2,6- dioxopiperidin-3-yl)-1,3-dioxoisoindolin-5- yl)piperidin-3-yl)amino)methyl)piperidin-1- yl)phenyl)-1H-indazol-3-yl)-2-methylbenzyl)- 1,2,4-oxadiazole-5-carboxamide |
| | 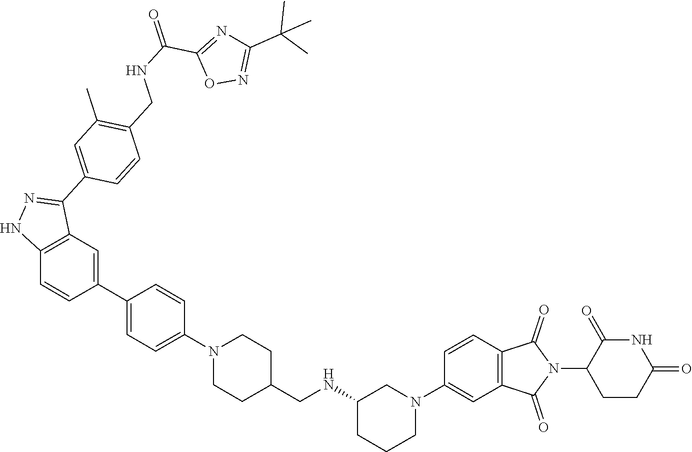 [SMILES: Cc1cc(-c2n[nH]c3ccc(-c4ccc(N5CCC(CN[C@H]6CCCN(c7ccc8c(c7)C(=O)N(C7CCC(=O)NC7=O)C8=O)C6)CC5)cc4)cc23)ccc1CNC(=O)c1nc(C(C)(C)C)no1] | 3-(tert-butyl)-N-(4-(5-(4-(4-((((3S)-1-(2-(2,6- dioxopiperidin-3-yl)-1,3-dioxoisoindolin-5- yl)piperidin-3-yl)amino)methyl)piperidin-1- yl)phenyl)-1H-indazol-3-yl)-2-methylbenzyl)- 1,2,4-oxadiazole-5-carboxamide |
| | 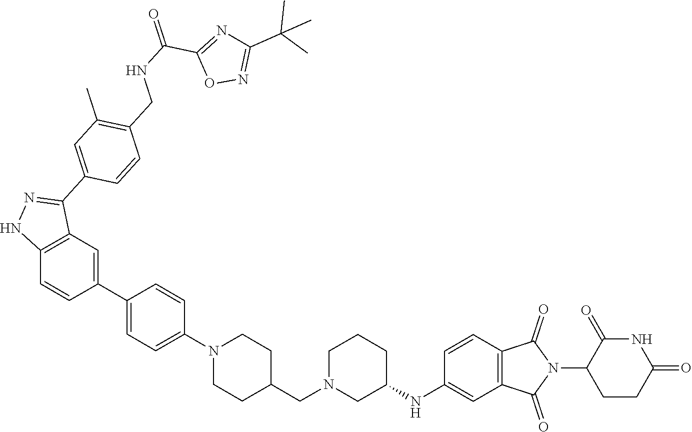 [SMILES: Cc1cc(-c2n[nH]c3ccc(-c4ccc(N5CCC(CN6CCC[C@H](Nc7ccc8c(c7)C(=O)N(C7CCC(=O)NC7=O)C8=O)C6)CC5)cc4)cc23)ccc1CNC(=O)c1nc(C(C)(C)C)no1] | 3-(tert-butyl)-N-(4-(5-(4-(4-(((3S)-3-((2-(2,6- dioxopiperidin-3-yl)-1,3-dioxoisoindolin-5- yl)amino)piperidin-1-yl)methyl)piperidin-1- yl)phenyl)-1H-indazol-3-yl)-2-methylbenzyl)- 1,2,4-oxadiazole-5-carboxamide |
| | 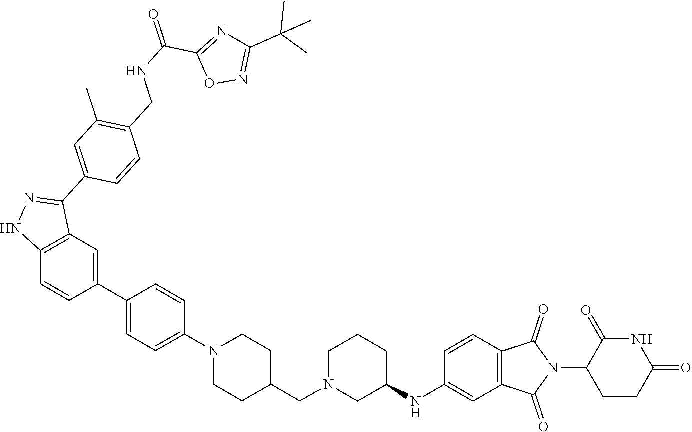 [SMILES: Cc1cc(-c2n[nH]c3ccc(-c4ccc(N5CCC(CN6CCC[C@@H](Nc7ccc8c(c7)C(=O)N(C7CCC(=O)NC7=O)C8=O)C6)CC5)cc4)cc23)ccc1CNC(=O)c1nc(C(C)(C)C)no1] | 3-(tert-butyl)-N-(4-(5-(4-(4-(((3R)-3-((2-(2,6- dioxopiperidin-3-yl)-1,3-dioxoisoindolin-5- yl)amino)piperidin-1-yl)methyl)piperidin-1- yl)phenyl)-1H-indazol-3-yl)-2-methylbenzyl)- 1,2,4-oxadiazole-5-carboxamide |
| | 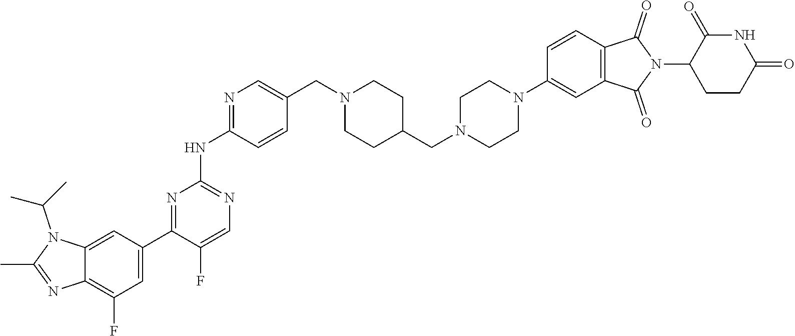 [SMILES: Cc1nc2c(F)cc(-c3nc(Nc4ccc(CN5CCC(CN6CCN(c7ccc8c(c7)C(=O)N(C7CCC(=O)NC7=O)C8=O)CC6)CC5)cn4)ncc3F)cc2n1C(C)C] | 2-(2,6-dioxopiperidin-3-yl)-5-(4-((1-((6-((5- fluoro-4-(4-fluoro-1-isopropyl-2-methyl-1H- benzo[d]imidazol-6-yl)pyrimidin-2- yl)amino)pyridin-3-yl)methyl)piperidin-4- yl)methyl)piperazin-1-yl)isoindoline-1,3-dione |
| | 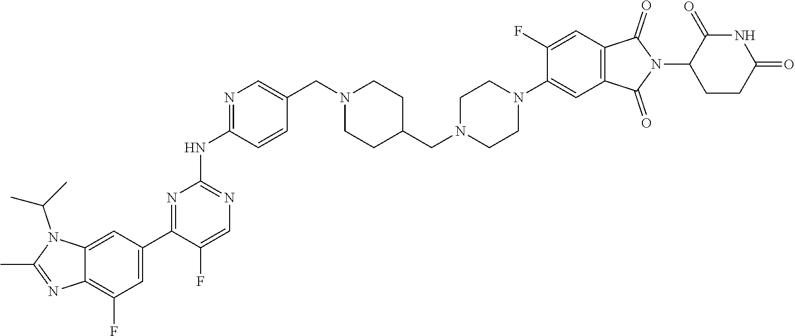 [SMILES: Cc1nc2c(F)cc(-c3nc(Nc4ccc(CN5CCC(CN6CCN(c7cc8c(cc7F)C(=O)N(C7CCC(=O)NC7=O)C8=O)CC6)CC5)cn4)ncc3F)cc2n1C(C)C] | 2-(2,6-dioxopiperidin-3-yl)-5-fluoro-6-(4-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)piperazin-1-yl)isoindoline-1,3-dione |
| | 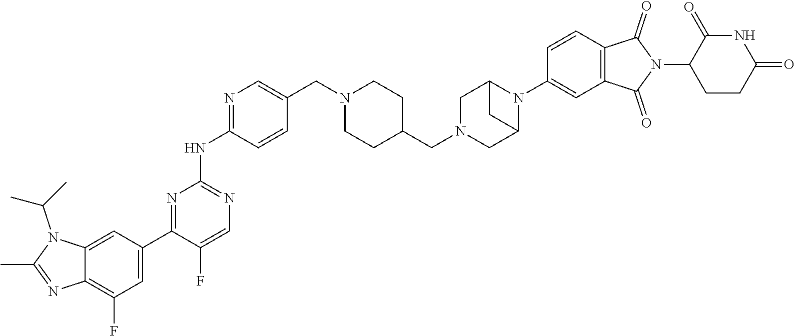 [SMILES: Cc1nc2c(F)cc(-c3nc(Nc4ccc(CN5CCC(CN6CC7CC(C6)N7c6ccc7c(c6)C(=O)N(C6CCC(=O)NC6=O)C7=O)CC5)cn4)ncc3F)cc2n1C(C)C] | 2-(2,6-dioxopiperidin-3-yl)-5-(3-((1-((6-((5- fluoro-4-(4-fluoro-1-isopropyl-2-methyl-1H- benzo[d]imidazol-6-yl)pyrimidin-2- yl)amino)pyridin-3-yl)methyl)piperidin-4- yl)methyl)-3,6-diazabicyclo[3.1.1]heptan-6- yl)isoindoline-1,3-dione |
| | 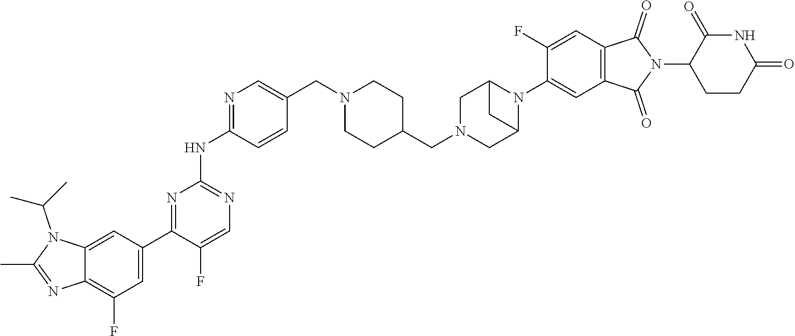 [SMILES: Cc1nc2c(F)cc(-c3nc(Nc4ccc(CN5CCC(CN6CC7CC(C6)N7c6cc7c(cc6F)C(=O)N(C6CCC(=O)NC6=O)C7=O)CC5)cn4)ncc3F)cc2n1C(C)C] | 2-(2,6-dioxopiperidin-3-yl)-5-fluoro-6-(3-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)-3,6-diazabicyclo[3.1.1]heptan-6- yl)isoindoline-1,3-dione |
| | 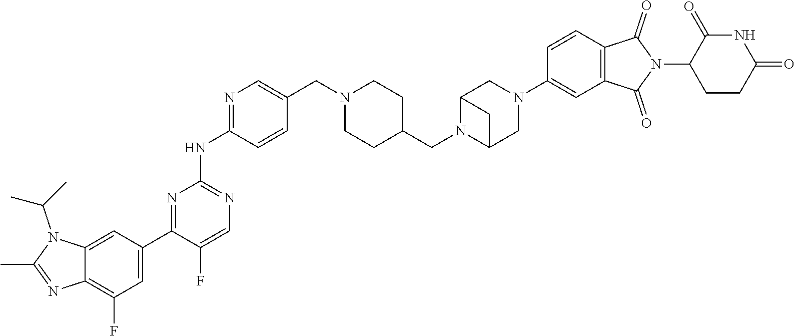 [SMILES: Cc1nc2c(F)cc(-c3nc(Nc4ccc(CN5CCC(CN6C7CC6CN(c6ccc8c(c6)C(=O)N(C6CCC(=O)NC6=O)C8=O)C7)CC5)cn4)ncc3F)cc2n1C(C)C] | 2-(2,6-dioxopiperidin-3-yl)-5-(6-((1-((6-((5- fluoro-4-(4-fluoro-1-isopropyl-2-methyl-1H- benzo[d]imidazol-6-yl)pyrimidin-2- yl)amino)pyridin-3-yl)methyl)piperidin-4- yl)methyl)-3,6-diazabicyclo[3.1.1]heptan-3- yl)isoindoline-1,3-dione |
| | 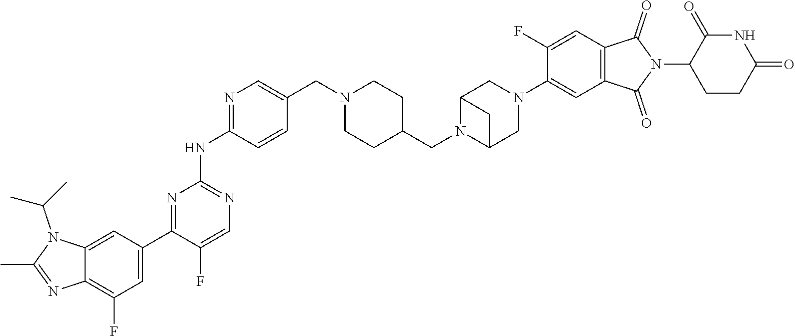 [SMILES: Cc1nc2c(F)cc(-c3nc(Nc4ccc(CN5CCC(CN6C7CC6CN(c6cc8c(cc6F)C(=O)N(C6CCC(=O)NC6=O)C8=O)C7)CC5)cn4)ncc3F)cc2n1C(C)C] | 2-(2,6-dioxopiperidin-3-yl)-5-fluoro-6-(6-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)-3,6-diazabicyclo[3.1.1]heptan-3- yl)isoindoline-1,3-dione |
| | 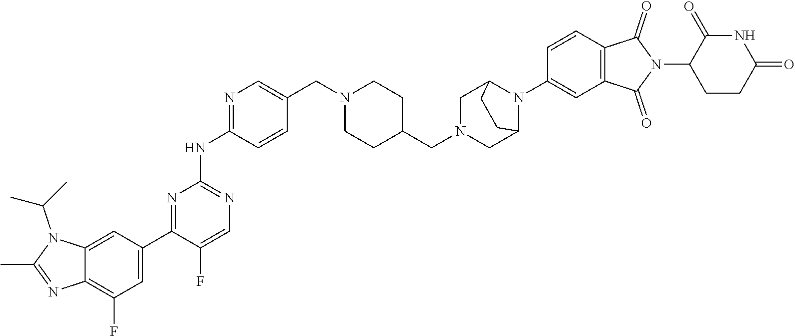 [SMILES: Cc1nc2c(F)cc(-c3nc(Nc4ccc(CN5CCC(CN6CC7CCC(C6)N7c6ccc7c(c6)C(=O)N(C6CCC(=O)NC6=O)C7=O)CC5)cn4)ncc3F)cc2n1C(C)C] | 2-(2,6-dioxopiperidin-3-yl)-5-(3-((1-((6-((5- fluoro-4-(4-fluoro-1-isopropyl-2-methyl-1H- benzo[d]imidazol-6-yl)pyrimidin-2- yl)amino)pyridin-3-yl)methyl)piperidin-4- yl)methyl)-3,8-diazabicyclo[3.2.1]octan-8- yl)isoindoline-1,3-dione |
| | 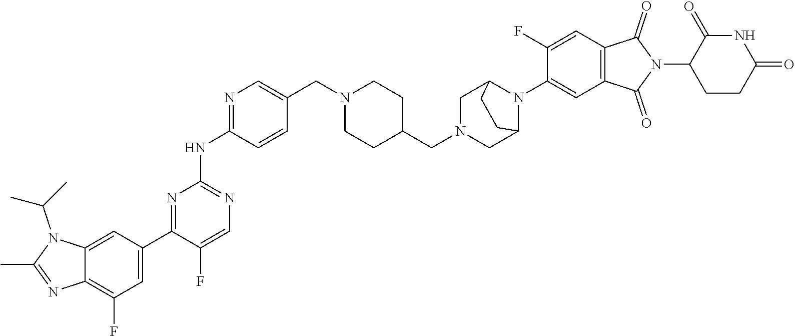 [SMILES: Cc1nc2c(F)cc(-c3nc(Nc4ccc(CN5CCC(CN6CC7CCC(C6)N7c6cc7c(cc6F)C(=O)N(C6CCC(=O)NC6=O)C7=O)CC5)cn4)ncc3F)cc2n1C(C)C] | 2-(2,6-dioxopiperidin-3-yl)-5-fluoro-6-(3-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)-3,8-diazabicyclo[3.2.1]octan-8- yl)isoindoline-1,3-dione |
| | 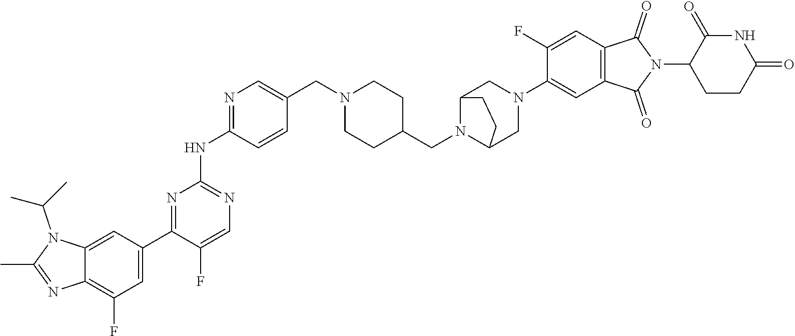 [SMILES: Cc1nc2c(F)cc(-c3nc(Nc4ccc(CN5CCC(CN6C7CCC6CN(c6cc8c(cc6F)C(=O)N(C6CCC(=O)NC6=O)C8=O)C7)CC5)cn4)ncc3F)cc2n1C(C)C] | 2-(2,6-dioxopiperidin-3-yl)-5-fluoro-6-(8-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)-3,8-diazabicyclo[3.2.1]octan-3- yl)isoindoline-1,3-dione |
| | 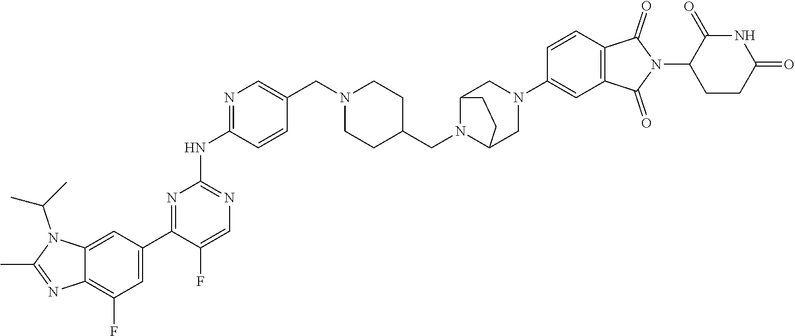 [SMILES: Cc1nc2c(F)cc(-c3nc(Nc4ccc(CN5CCC(CN6C7CCC6CN(c6ccc8c(c6)C(=O)N(C6CCC(=O)NC6=O)C8=O)C7)CC5)cn4)ncc3F)cc2n1C(C)C] | 2-(2,6-dioxopiperidin-3-yl)-5-(8-((1-((6-((5- fluoro-4-(4-fluoro-1-isopropyl-2-methyl-1H- benzo[d]imidazol-6-yl)pyrimidin-2- yl)amino)pyridin-3-yl)methyl)piperidin-4- yl)methyl)-3,8-diazabicyclo[3.2.1]octan-3- yl)isoindoline-1,3-dione |
| | 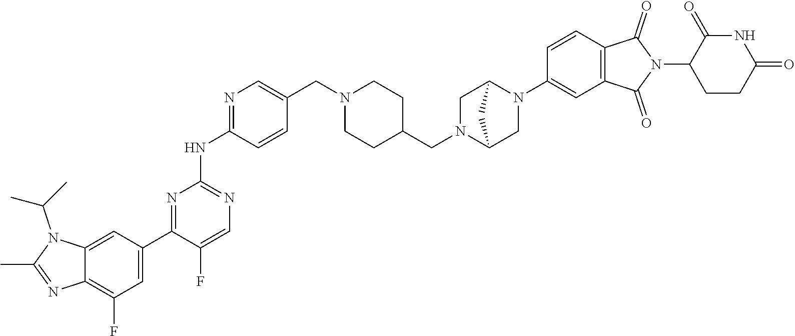 [SMILES: Cc1nc2c(F)cc(-c3nc(Nc4ccc(CN5CCC(CN6C[C@@H]7C[C@H]6CN7c6ccc7c(c6)C(=O)N(C6CCC(=O)NC6=O)C7=O)CC5)cn4)ncc3F)cc2n1C(C)C] | 2-(2,6-dioxopiperidin-3-yl)-5-((1S,4S)-5-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)-2,5-diazabicyclo[2.2.1]heptan-2- yl)isoindoline-1,3-dione |
| | 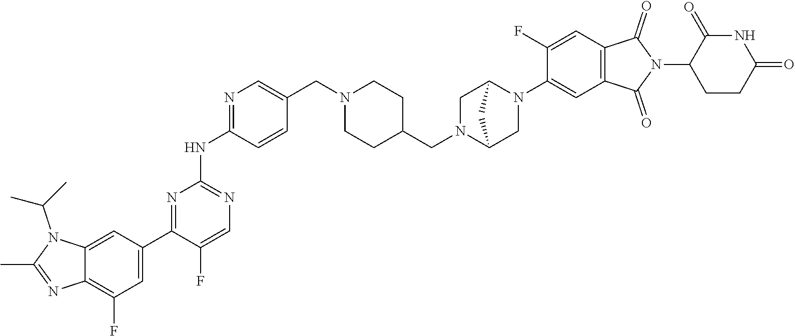 [SMILES: Cc1nc2c(F)cc(-c3nc(Nc4ccc(CN5CCC(CN6C[C@@H]7C[C@H]6CN7c6cc7c(cc6F)C(=O)N(C6CCC(=O)NC6=O)C7=O)CC5)cn4)ncc3F)cc2n1C(C)C] | 2-(2,6-dioxopiperidin-3-yl)-5-fluoro-6- ((1S,4S)-5-((1-((6-((5-fluoro-4-(4-fluoro-1- isopropyl-2-methyl-1H-benzo[d]imidazol-6- yl)pyrimidin-2-yl)amino)pyridin-3- yl)methyl)piperidin-4-yl)methyl)-2,5- diazabicyclo[2.2.1]heptan-2-yl)isoindoline-1,3- dione |
| | 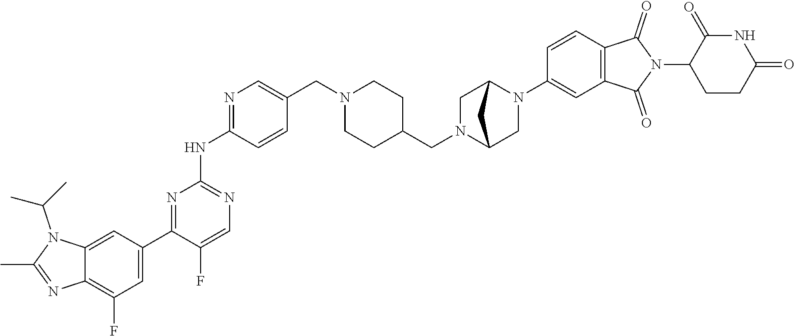 [SMILES: Cc1nc2c(F)cc(-c3nc(Nc4ccc(CN5CCC(CN6C[C@H]7C[C@@H]6CN7c6ccc7c(c6)C(=O)N(C6CCC(=O)NC6=O)C7=O)CC5)cn4)ncc3F)cc2n1C(C)C] | 2-(2,6-dioxopiperidin-3-yl)-5-((1R,4R)-5-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)-2,5-diazabicyclo[2.2.1]heptan-2- yl)isoindoline-1,3-dione |
| | 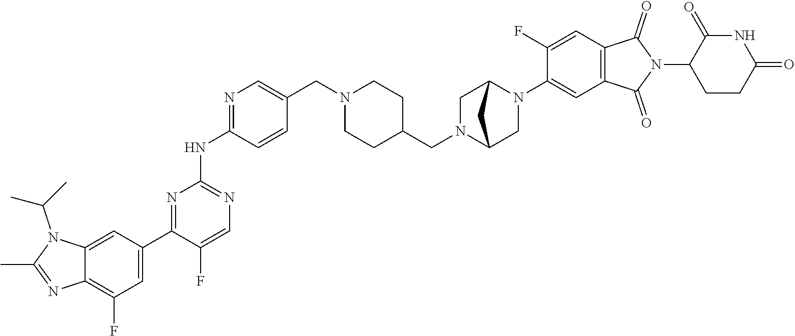 [SMILES: Cc1nc2c(F)cc(-c3nc(Nc4ccc(CN5CCC(CN6C[C@H]7C[C@@H]6CN7c6cc7c(cc6F)C(=O)N(C6CCC(=O)NC6=O)C7=O)CC5)cn4)ncc3F)cc2n1C(C)C] | 2-(2,6-dioxopiperidin-3-yl)-5-fluoro-6- ((1R,4R)-5-((1-((6-((5-fluoro-4-(4-fluoro-1- isopropyl-2-methyl-1H-benzo[d]imidazol-6- yl)pyrimidin-2-yl)amino)pyridin-3- yl)methyl)piperidin-4-yl)methyl)-2,5- diazabicyclo[2.2.1]heptan-2-yl)isoindoline-1,3- dione |
| | 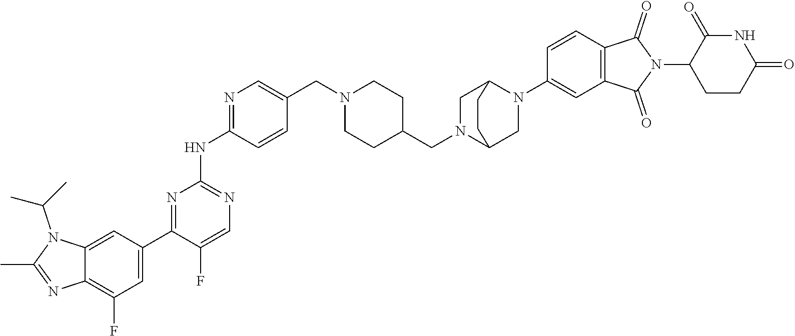 [SMILES: Cc1nc2c(F)cc(-c3nc(Nc4ccc(CN5CCC(CN6CC7CCC6CN7c6ccc7c(c6)C(=O)N(C6CCC(=O)NC6=O)C7=O)CC5)cn4)ncc3F)cc2n1C(C)C] | 2-(2,6-dioxopiperidin-3-yl)-5-(5-((1-((6-((5- fluoro-4-(4-fluoro-1-isopropyl-2-methyl-1H- benzo[d]imidazol-6-yl)pyrimidin-2- yl)amino)pyridin-3-yl)methyl)piperidin-4- yl)methyl)-2,5-diazabicyclo[2.2.2]octan-2- yl)isoindoline-1,3-dione |
| | 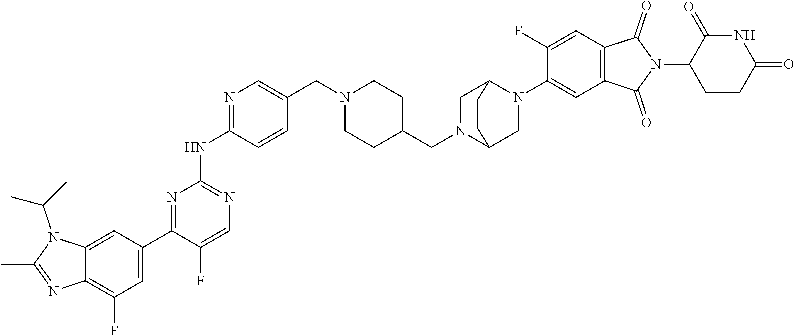 [SMILES: Cc1nc2c(F)cc(-c3nc(Nc4ccc(CN5CCC(CN6CC7CCC6CN7c6cc7c(cc6F)C(=O)N(C6CCC(=O)NC6=O)C7=O)CC5)cn4)ncc3F)cc2n1C(C)C] | 2-(2,6-dioxopiperidin-3-yl)-5-fluoro-6-(5-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)-2,5-diazabicyclo[2.2.2]octan-2- yl)isoindoline-1,3-dione |
| | 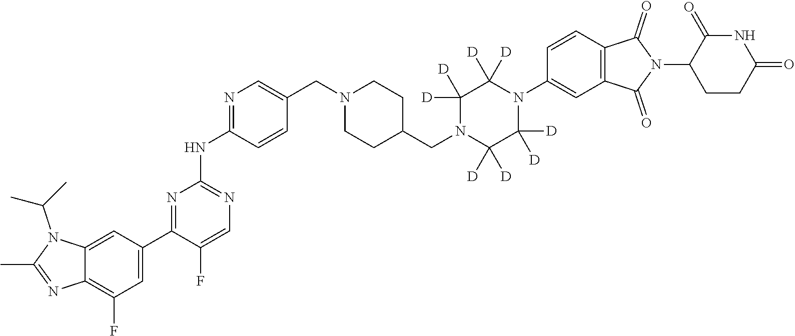 [SMILES: [2H]C1([2H])N(CC2CCN(Cc3ccc(Nc4ncc(F)c(-c5cc(F)c6nc(C)n(C(C)C)c6c5)n4)nc3)CC2)C([2H])([2H])C([2H])([2H])N(c2ccc3c(c2)C(=O)N(C2CCC(=O)NC2=O)C3=O)C1([2H])[2H]] | 2-(2,6-dioxopiperidin-3-yl)-5-(4-((1-((6-((5- fluoro-4-(4-fluoro-1-isopropyl-2-methyl-1H- benzo[d]imidazol-6-yl)pyrimidin-2- yl)amino)pyridin-3-yl)methyl)piperidin-4- yl)methyl)piperazin-1-yl-2,2,3,3,5,5,6,6- ds)isoindoline-1,3-dione |
| | 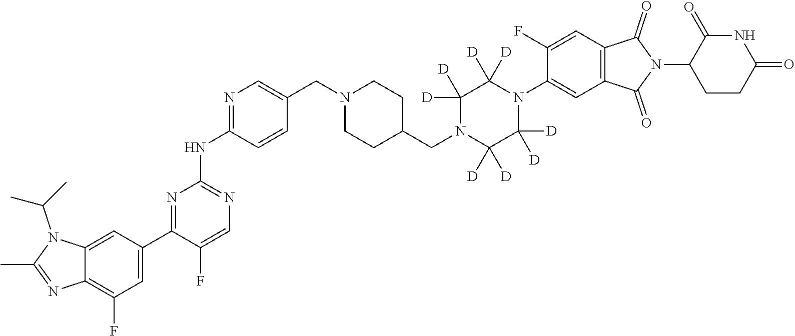 [SMILES: [2H]C1([2H])N(CC2CCN(Cc3ccc(Nc4ncc(F)c(-c5cc(F)c6nc(C)n(C(C)C)c6c5)n4)nc3)CC2)C([2H])([2H])C([2H])([2H])N(c2cc3c(cc2F)C(=O)N(C2CCC(=O)NC2=O)C3=O)C1([2H])[2H]] | 2-(2,6-dioxopiperidin-3-yl)-5-fluoro-6-(4-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)piperazin-1-yl-2,2,3,3,5,5,6,6- d₈)isoindoline-1,3-dione |
| | 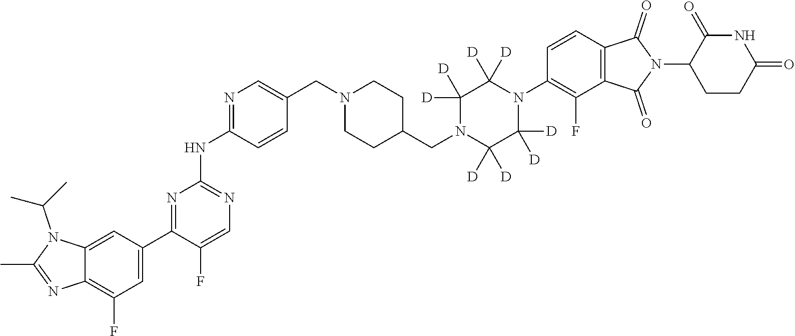 [SMILES: [2H]C1([2H])N(CC2CCN(Cc3ccc(Nc4ncc(F)c(-c5cc(F)c6nc(C)n(C(C)C)c6c5)n4)nc3)CC2)C([2H])([2H])C([2H])([2H])N(c2ccc3c(c2F)C(=O)N(C2CCC(=O)NC2=O)C3=O)C1([2H])[2H]] | 2-(2,6-dioxopiperidin-3-yl)-4-fluoro-5-(4-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)piperazin-1-yl-2,2,3,3,5,5,6,6- d₈)isoindoline-1,3-dione |
| | 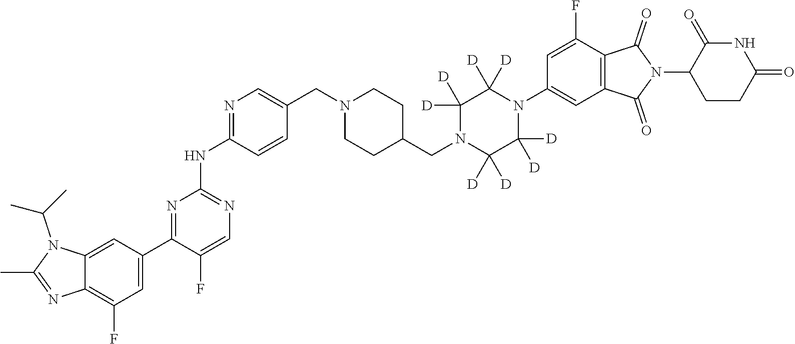 [SMILES: [2H]C1([2H])N(CC2CCN(Cc3ccc(Nc4ncc(F)c(-c5cc(F)c6nc(C)n(C(C)C)c6c5)n4)nc3)CC2)C([2H])([2H])C([2H])([2H])N(c2cc(F)c3c(c2)C(=O)N(C2CCC(=O)NC2=O)C3=O)C1([2H])[2H]] | 2-(2,6-dioxopiperidin-3-yl)-4-fluoro-6-(4-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)piperazin-1-yl-2,2,3,3,5,5,6,6- d₈)isoindoline-1,3-dione |
| | 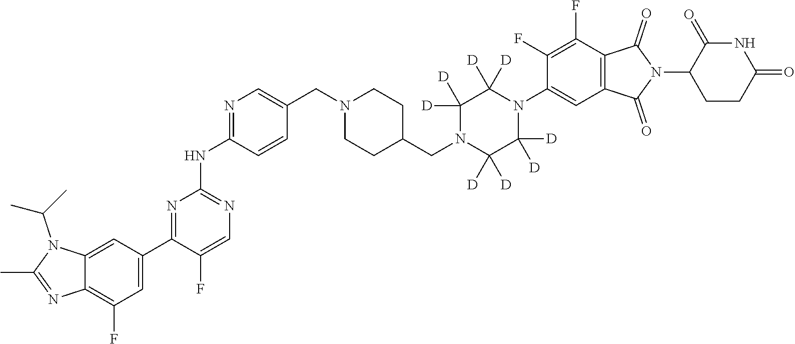 [SMILES: [2H]C1([2H])N(CC2CCN(Cc3ccc(Nc4ncc(F)c(-c5cc(F)c6nc(C)n(C(C)C)c6c5)n4)nc3)CC2)C([2H])([2H])C([2H])([2H])N(c2cc3c(c(F)c2F)C(=O)N(C2CCC(=O)NC2=O)C3=O)C1([2H])[2H]] | 2-(2,6-dioxopiperidin-3-yl)-4,5-difluoro-6-(4- ((1-((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)piperazin-1-yl-2,2,3,3,5,5,6,6- d₈)isoindoline-1,3-dione |
| | 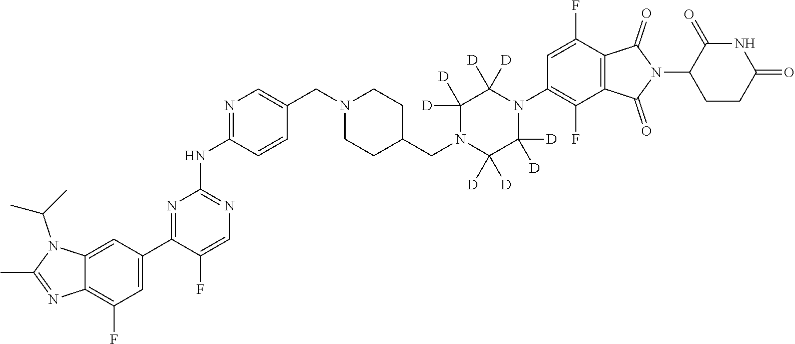 [SMILES: [2H]C1([2H])N(CC2CCN(Cc3ccc(Nc4ncc(F)c(-c5cc(F)c6nc(C)n(C(C)C)c6c5)n4)nc3)CC2)C([2H])([2H])C([2H])([2H])N(c2cc(F)c3c(c2F)C(=O)N(C2CCC(=O)NC2=O)C3=O)C1([2H])[2H]] | 2-(2,6-dioxopiperidin-3-yl)-4,7-difluoro-5-(4- ((1-((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)piperazin-1-yl-2,2,3,3,5,5,6,6- d₈)isoindoline-1,3-dione |
| | 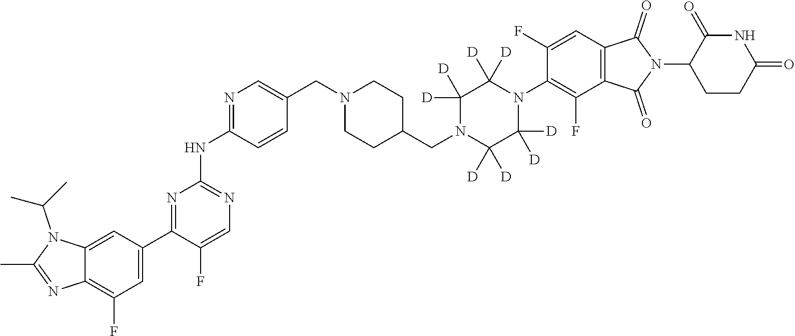 [SMILES: [2H]C1([2H])N(CC2CCN(Cc3ccc(Nc4ncc(F)c(-c5cc(F)c6nc(C)n(C(C)C)c6c5)n4)nc3)CC2)C([2H])([2H])C([2H])([2H])N(c2c(F)cc3c(c2F)C(=O)N(C2CCC(=O)NC2=O)C3=O)C1([2H])[2H]] | 2-(2,6-dioxopiperidin-3-yl)-4,6-difluoro-5-(4- ((1-((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)piperazin-1-yl-2,2,3,3,5,5,6,6- d₈)isoindoline-1,3-dione |
| | 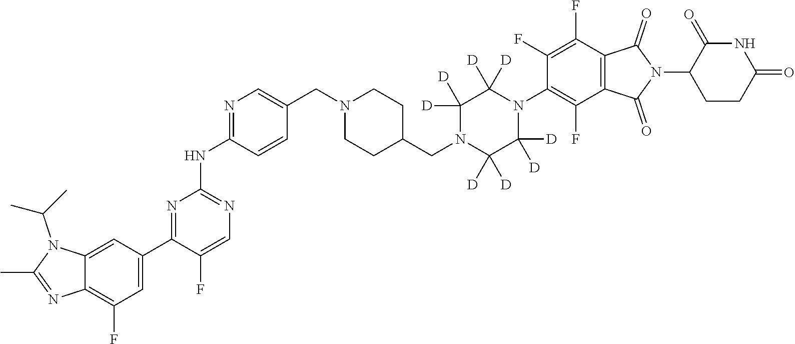 [SMILES: [2H]C1([2H])N(CC2CCN(Cc3ccc(Nc4ncc(F)c(-c5cc(F)c6nc(C)n(C(C)C)c6c5)n4)nc3)CC2)C([2H])([2H])C([2H])([2H])N(c2c(F)c(F)c3c(c2F)C(=O)N(C2CCC(=O)NC2=O)C3=O)C1([2H])[2H]] | 2-(2,6-dioxopiperidin-3-yl)-4,5,7-trifluoro-6- (4-((1-((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)piperazin-1-yl-2,2,3,3,5,5,6,6- d₈)isoindoline-1,3-dione |
| | 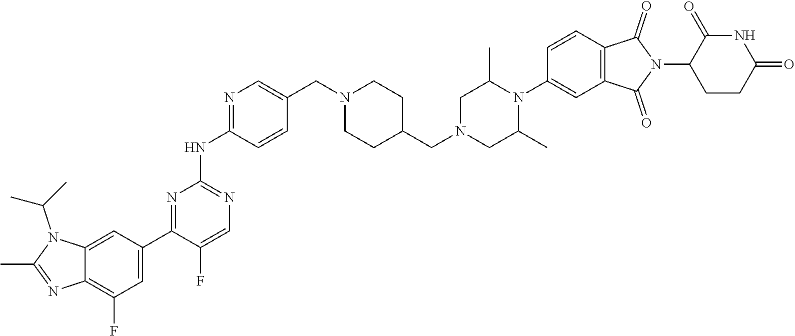 [SMILES: Cc1nc2c(F)cc(-c3nc(Nc4ccc(CN5CCC(CN6CC(C)N(c7ccc8c(c7)C(=O)N(C7CCC(=O)NC7=O)C8=O)C(C)C6)CC5)cn4)ncc3F)cc2n1C(C)C] | 2-(2,6-dioxopiperidin-3-yl)-5-(4-((1-((6-((5- fluoro-4-(4-fluoro-1-isopropyl-2-methyl-1H- benzo[d]imidazol-6-yl)pyrimidin-2- yl)amino)pyridin-3-yl)methyl)piperidin-4- yl)methyl)-2,6-dimethylpiperazin-1- yl)isoindoline-1,3-dione |
| | 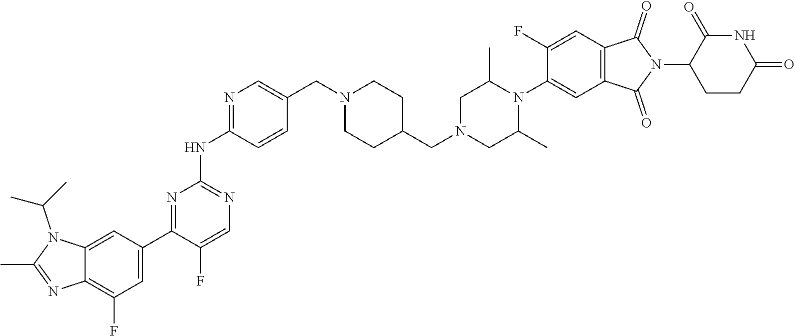 [SMILES: Cc1nc2c(F)cc(-c3nc(Nc4ccc(CN5CCC(CN6CC(C)N(c7cc8c(cc7F)C(=O)N(C7CCC(=O)NC7=O)C8=O)C(C)C6)CC5)cn4)ncc3F)cc2n1C(C)C] | 2-(2,6-dioxopiperidin-3-yl)-5-fluoro-6-(4-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)-2,6-dimethylpiperazin-1- yl)isoindoline-1,3-dione |
| | 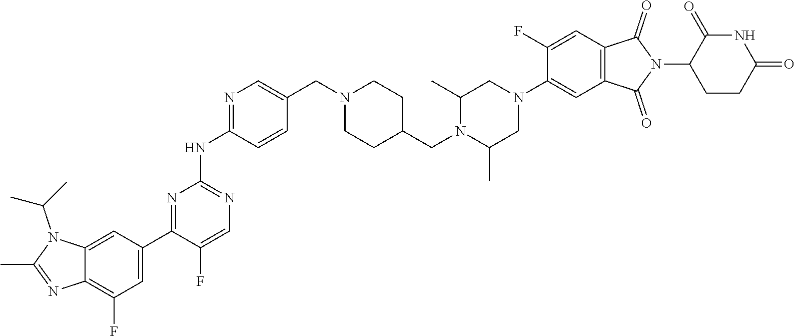 [SMILES: Cc1nc2c(F)cc(-c3nc(Nc4ccc(CN5CCC(CN6C(C)CN(c7cc8c(cc7F)C(=O)N(C7CCC(=O)NC7=O)C8=O)CC6C)CC5)cn4)ncc3F)cc2n1C(C)C] | 2-(2,6-dioxopiperidin-3-yl)-5-fluoro-6-(4-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)-3,5-dimethylpiperazin-1- yl)isoindoline-1,3-dione |
| | 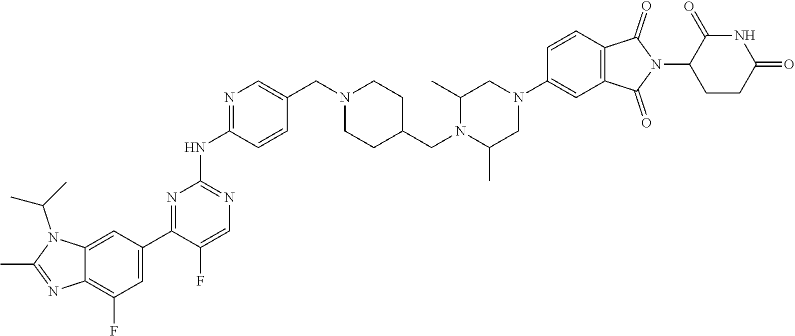 [SMILES: Cc1nc2c(F)cc(-c3nc(Nc4ccc(CN5CCC(CN6C(C)CN(c7ccc8c(c7)C(=O)N(C7CCC(=O)NC7=O)C8=O)CC6C)CC5)cn4)ncc3F)cc2n1C(C)C] | 2-(2,6-dioxopiperidin-3-yl)-5-(4-((1-((6-((5- fluoro-4-(4-fluoro-1-isopropyl-2-methyl-1H- benzo[d]imidazol-6-yl)pyrimidin-2- yl)amino)pyridin-3-yl)methyl)piperidin-4- yl)methyl)-3,5-dimethylpiperazin-1- yl)isoindoline-1,3-dione |
| | 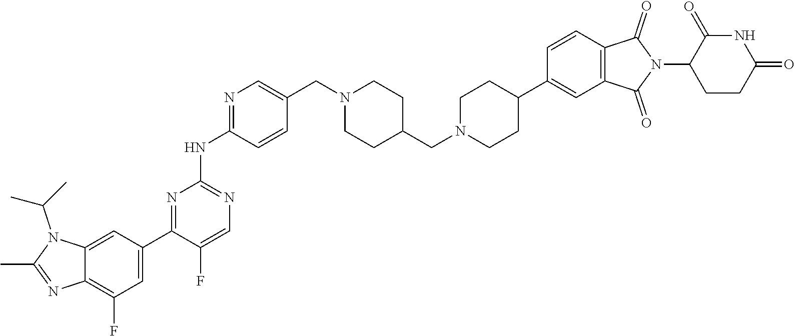 [SMILES: Cc1nc2c(F)cc(-c3nc(Nc4ccc(CN5CCC(CN6CCC(c7ccc8c(c7)C(=O)N(C7CCC(=O)NC7=O)C8=O)CC6)CC5)cn4)ncc3F)cc2n1C(C)C] | 2-(2,6-dioxopiperidin-3-yl)-5-(1-((1-((6-((5- fluoro-4-(4-fluoro-1-isopropyl-2-methyl-1H- benzo[d]imidazol-6-yl)pyrimidin-2- yl)amino)pyridin-3-yl)methyl)piperidin-4- yl)methyl)piperidin-4-yl)isoindoline-1,3-dione |
| | 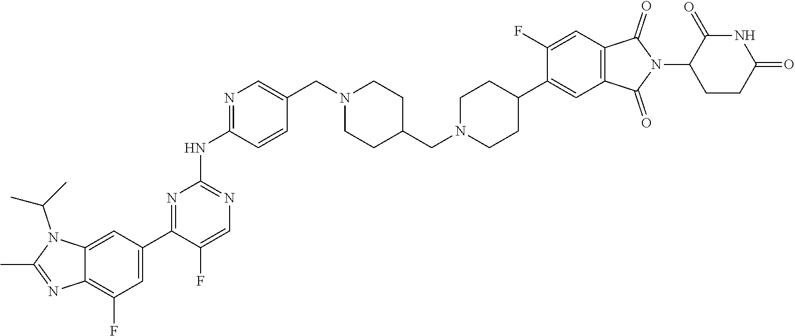 [SMILES: Cc1nc2c(F)cc(-c3nc(Nc4ccc(CN5CCC(CN6CCC(c7cc8c(cc7F)C(=O)N(C7CCC(=O)NC7=O)C8=O)CC6)CC5)cn4)ncc3F)cc2n1C(C)C] | 2-(2,6-dioxopiperidin-3-yl)-5-fluoro-6-(1-((1- ((6-((5-fluoro-4-(4-fluoro-1-isopropyl-2- methyl-1H-benzo[d]imidazol-6-yl)pyrimidin-2- yl)amino)pyridin-3-yl)methyl)piperidin-4- yl)methyl)piperidin-4-yl)isoindoline-1,3-dione |
| | 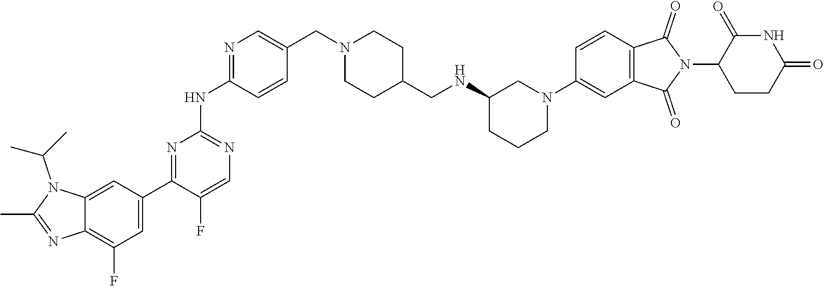 [SMILES: Cc1nc2c(F)cc(-c3nc(Nc4ccc(CN5CCC(CN[C@@H]6CCCN(c7ccc8c(c7)C(=O)N(C7CCC(=O)NC7=O)C8=O)C6)CC5)cn4)ncc3F)cc2n1C(C)C] | 2-(2,6-dioxopiperidin-3-yl)-5-((R)-3-(((1-((6- ((5-fluoro-4-(4-fluoro-1-isopropyl-2-methyl- 1H-benzo[d]imidazol-6-yl)pyrimidin-2- yl)amino)pyridin-3-yl)methyl)piperidin-4- yl)methyl)amino)piperidin-1-yl)isoindoline- 1,3-dione |
| | 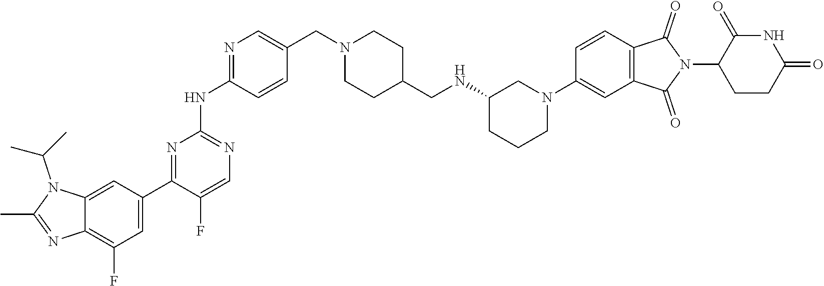 [SMILES: Cc1nc2c(F)cc(-c3nc(Nc4ccc(CN5CCC(CN[C@H]6CCCN(c7ccc8c(c7)C(=O)N(C7CCC(=O)NC7=O)C8=O)C6)CC5)cn4)ncc3F)cc2n1C(C)C] | 2-(2,6-dioxopiperidin-3-yl)-5-((S)-3-(((1-((6- ((5-fluoro-4-(4-fluoro-1-isopropyl-2-methyl- 1H-benzo[d]imidazol-6-yl)pyrimidin-2- yl)amino)pyridin-3-yl)methyl)piperidin-4- yl)methyl)amino)piperidin-1-yl)isoindoline- 1,3-dione |
| | 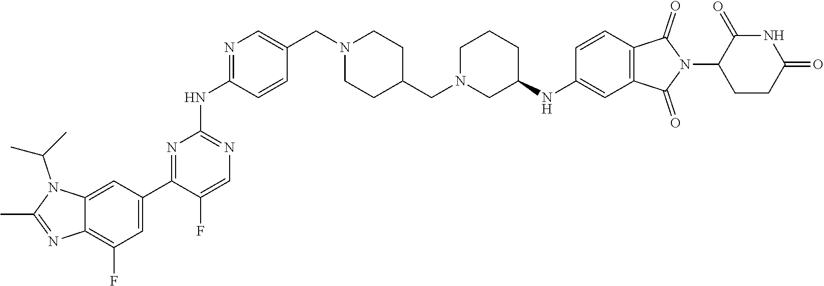 [SMILES: Cc1nc2c(F)cc(-c3nc(Nc4ccc(CN5CCC(CN6CCC[C@@H](Nc7ccc8c(c7)C(=O)N(C7CCC(=O)NC7=O)C8=O)C6)CC5)cn4)ncc3F)cc2n1C(C)C] | 2-(2,6-dioxopiperidin-3-yl)-5-(((R)-1-((1-((6- ((5-fluoro-4-(4-fluoro-1-isopropyl-2-methyl- 1H-benzo[d]imidazol-6-yl)pyrimidin-2- yl)amino)pyridin-3-yl)methyl)piperidin-4- yl)methyl)piperidin-3-yl)amino)isoindoline- 1,3-dione |
| | 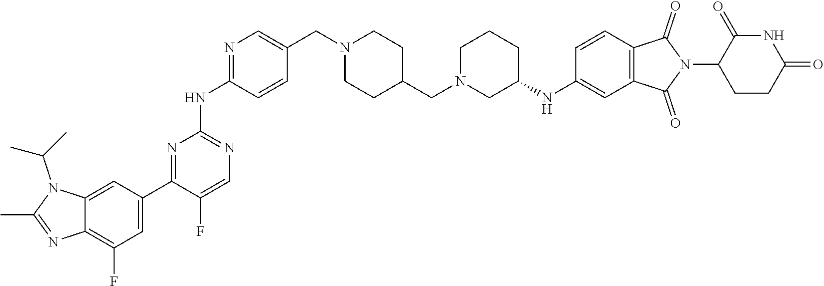 [SMILES: Cc1nc2c(F)cc(-c3nc(Nc4ccc(CN5CCC(CN6CCC[C@H](Nc7ccc8c(c7)C(=O)N(C7CCC(=O)NC7=O)C8=O)C6)CC5)cn4)ncc3F)cc2n1C(C)C] | 2-(2,6-dioxopiperidin-3-yl)-5-(((S)-1-((1-((6- ((5-fluoro-4-(4-fluoro-1-isopropyl-2-methyl- 1H-benzo[d]imidazol-6-yl)pyrimidin-2- yl)amino)pyridin-3-yl)methyl)piperidin-4- yl)methyl)piperidin-3-yl)amino)isoindoline- 1,3-dione |
| GT-03660 | 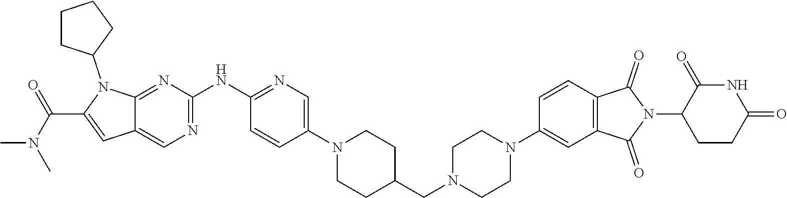 [SMILES: CN(C)C(=O)c1cc2cnc(Nc3ccc(N4CCC(CN5CCN(c6ccc7c(c6)C(=O)N(C6CCC(=O)NC6=O)C7=O)CC5)CC4)cn3)nc2n1C1CCCC1] | 7-cyclopentyl-2-((5-(4-((4-(2-(2,6- dioxopiperidin-3-yl)-1,3-dioxoisoindolin-5- yl)piperazin-1-yl)methyl)piperidin-1- yl)pyridin-2-yl)amino)-N,N-dimethyl-7H- pyrrolo[2,3-d]pyrimidine-6-carboxamide |
| GT-03656 | 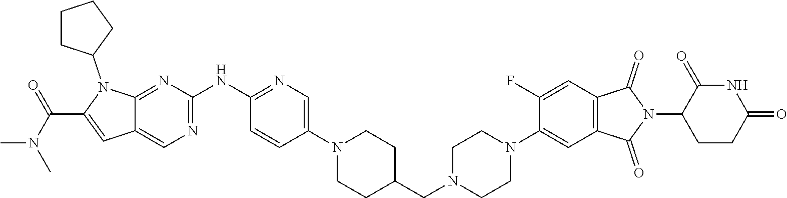 [SMILES: CN(C)C(=O)c1cc2cnc(Nc3ccc(N4CCC(CN5CCN(c6cc7c(cc6F)C(=O)N(C6CCC(=O)NC6=O)C7=O)CC5)CC4)cn3)nc2n1C1CCCC1] | 7-cyclopentyl-2-((5-(4-((4-(2-(2,6- dioxopiperidin-3-yl)-6-fluoro-1,3- dioxoisoindolin-5-yl)piperazin-1 - yl)methyl)piperidin-1-yl)pyridin-2-yl)amino)- N,N-dimethyl-7H-pyrrolo[2,3-d]pyrimidine-6- carboxamide |
| GT-03654 | 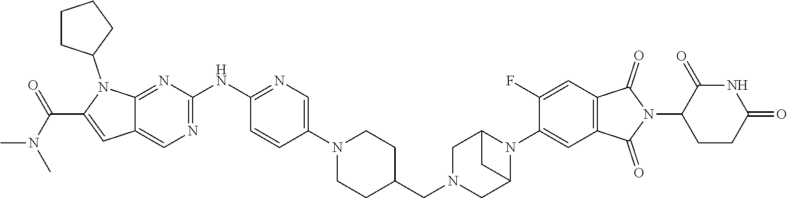 [SMILES: CN(C)C(=O)c1cc2cnc(Nc3ccc(N4CCC(CN5CC6CC(C5)N6c5cc6c(cc5F)C(=O)N(C5CCC(=O)NC5=O)C6=O)CC4)cn3)nc2n1C1CCCC1] | 7-cyclopentyl-2-((5-(4-((6-(2-(2,6- dioxopiperidin-3-yl)-6-fluoro-1,3- dioxoisoindolin-5-yl)-3,6- diazabicyclo[3.1.1]heptan-3- yl)methyl)piperidin-1-yl)pyridin-2-yl)amino)- N,N-dimethyl-7H-pyrrolo[2,3-d]pyrimidine-6- carboxamide |
| GT-03659 | 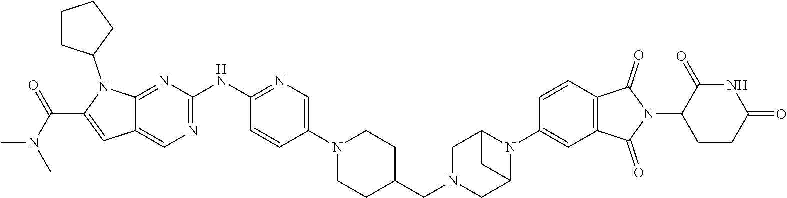 [SMILES: CN(C)C(=O)c1cc2cnc(Nc3ccc(N4CCC(CN5CC6CC(C5)N6c5ccc6c(c5)C(=O)N(C5CCC(=O)NC5=O)C6=O)CC4)cn3)nc2n1C1CCCC1] | 7-cyclopentyl-2-((5-(4-((6-(2-(2,6- dioxopiperidin-3-yl)-1,3-dioxoisoindolin-5-yl)- 3,6-diazabicyclo[3.1.1]heptan-3- yl)methyl)piperidin-1-yl)pyridin-2-yl)amino)- N,N-dimethyl-7H-pyrrolo[2,3-d]pyrimidine-6- carboxamide |
| GT-03661 | 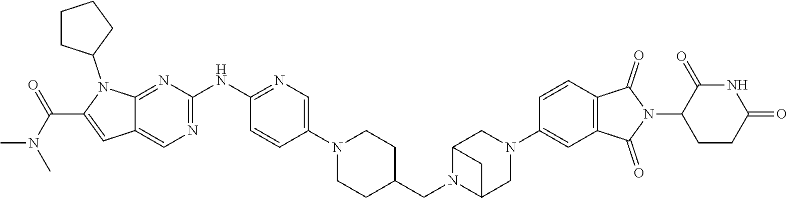 [SMILES: CN(C)C(=O)c1cc2cnc(Nc3ccc(N4CCC(CN5C6CC5CN(c5ccc7c(c5)C(=O)N(C5CCC(=O)NC5=O)C7=O)C6)CC4)cn3)nc2n1C1CCCC1] | 7-cyclopentyl-2-((5-(4-((3-(2-(2,6- dioxopiperidin-3-yl)-1,3-dioxoisoindolin-5-yl)- 3,6-diazabicyclo[3.1.1]heptan-6- yl)methyl)piperidin-1-yl)pyridin-2-yl)amino)- N,N-dimethyl-7H-pyrrolo[2,3-d]pyrimidine-6- carboxamide |
| GT-03657 | 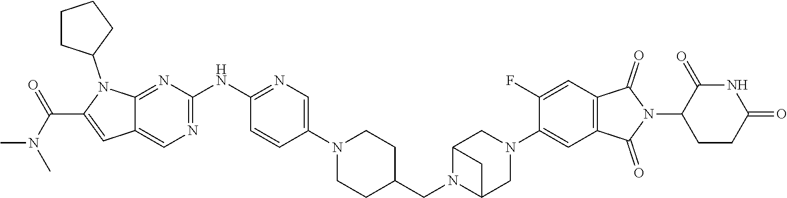 [SMILES: CN(C)C(=O)c1cc2cnc(Nc3ccc(N4CCC(CN5C6CC5CN(c5cc7c(cc5F)C(=O)N(C5CCC(=O)NC5=O)C7=O)C6)CC4)cn3)nc2n1C1CCCC1] | 7-cyclopentyl-2-((5-(4-((3-(2-(2,6- dioxopiperidin-3-yl)-6-fluoro-1,3- dioxoisoindolin-5-yl)-3,6- diazabicyclo[3.1.1]heptan-6- yl)methyl)piperidin-1-yl)pyridin-2-yl)amino)- N,N-dimethyl-7H-pyrrolo[2,3-d]pyrimidine-6- carboxamide |
| GT-03655 | 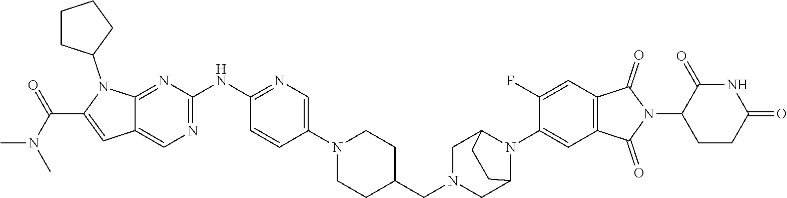 [SMILES: CN(C)C(=O)c1cc2cnc(Nc3ccc(N4CCC(CN5CC6CCC(C5)N6c5cc6c(cc5F)C(=O)N(C5CCC(=O)NC5=O)C6=O)CC4)cn3)nc2n1C1CCCC1] | 7-cyclopentyl-2-((5-(4-((8-(2-(2,6- dioxopiperidin-3-yl)-6-fluoro-1,3- dioxoisoindolin-5-yl)-3,8- diazabicyclo[3.2.1]octan-3-yl)methyl)piperidin- 1-yl)pyridin-2-yl)amino)-N,N-dimethyl-7H- pyrrolo[2,3-d]pyrimidine-6-carboxamide |
| GT-03651 | 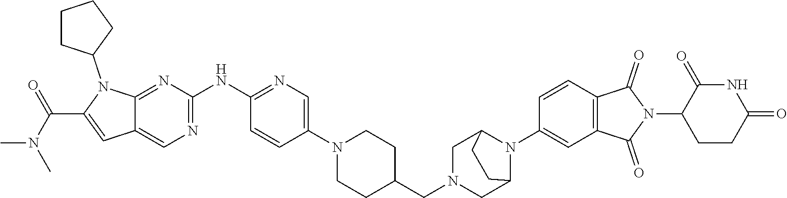 [SMILES: CN(C)C(=O)c1cc2cnc(Nc3ccc(N4CCC(CN5CC6CCC(C5)N6c5ccc6c(c5)C(=O)N(C5CCC(=O)NC5=O)C6=O)CC4)cn3)nc2n1C1CCCC1] | 7-cyclopentyl-2-((5-(4-((8-(2-(2,6- dioxopiperidin-3-yl)-1,3-dioxoisoindolin-5-yl)- 3,8-diazabicyclo[3.2.1]octan-3- yl)methyl)piperidin-1-yl)pyridin-2-yl)amino)- N,N-dimethyl-7H-pyrrolo[2,3-d]pyrimidine-6- carboxamide |
| GT-03652 | 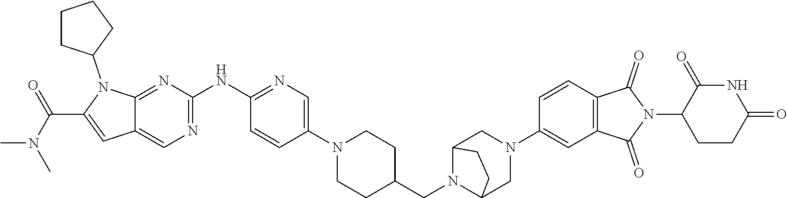 [SMILES: CN(C)C(=O)c1cc2cnc(Nc3ccc(N4CCC(CN5C6CCC5CN(c5ccc7c(c5)C(=O)N(C5CCC(=O)NC5=O)C7=O)C6)CC4)cn3)nc2n1C1CCCC1] | 7-cyclopentyl-2-((5-(4-((3-(2-(2,6- dioxopiperidin-3-yl)-1,3-dioxoisoindolin-5-yl)- 3,8-diazabicyclo[3.2.1]octan-8- yl)methyl)piperidin-1-yl)pyridin-2-yl)amino)- N,N-dimethyl-7H-pyrrolo[2,3-d]pyrimidine-6- carboxamide |
| GT-03658 | 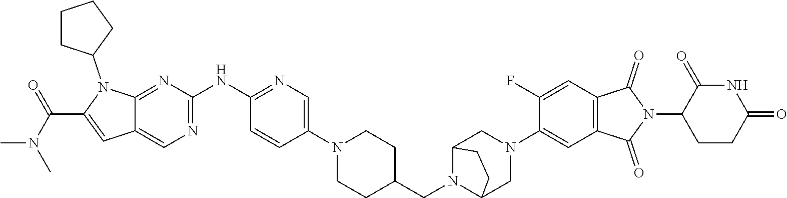 [SMILES: CN(C)C(=O)c1cc2cnc(Nc3ccc(N4CCC(CN5C6CCC5CN(c5cc7c(cc5F)C(=O)N(C5CCC(=O)NC5=O)C7=O)C6)CC4)cn3)nc2n1C1CCCC1] | 7-cyclopentyl-2-((5-(4-((3-(2-(2,6- dioxopiperidin-3-yl)-6-fluoro-1,3- dioxoisoindolin-5-yl)-3,8- diazabicyclo[3.2.1]octan-8-yl)methyl)piperidin- 1-yl)pyridin-2-yl)amino)-N,N-dimethyl-7H- pyrrolo[2,3-d]pyrimidine-6-carboxamide |
| | 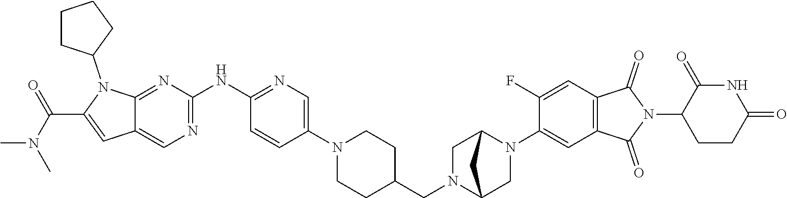 [SMILES: CN(C)C(=O)c1cc2cnc(Nc3ccc(N4CCC(CN5C[C@H]6C[C@@H]5CN6c5cc6c(cc5F)C(=O)N(C5CCC(=O)NC5=O)C6=O)CC4)cn3)nc2n1C1CCCC1] | 7-cyclopentyl-2-((5-(4-(((1R,4R)-5-(2-(2,6- dioxopiperidin-3-yl)-6-fluoro-1,3- dioxoisoindolin-5-yl)-2,5- diazabicyclo[2.2.1]heptan-2- yl)methyl)piperidin-1-yl)pyridin-2-yl)amino)- N,N-dimethyl-7H-pyrrolo[2,3-d]pyrimidine-6- carboxamide |
| | 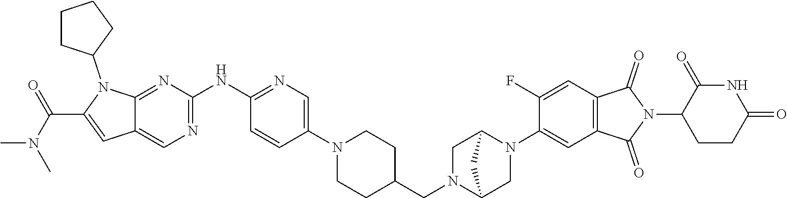 [SMILES: CN(C)C(=O)c1cc2cnc(Nc3ccc(N4CCC(CN5C[C@@H]6C[C@H]5CN6c5cc6c(cc5F)C(=O)N(C5CCC(=O)NC5=O)C6=O)CC4)cn3)nc2n1C1CCCC1] | 7-cyclopentyl-2-((5-(4-(((1S,4S)-5-(2-(2,6- dioxopiperidin-3-yl)-6-fluoro-1,3- dioxoisoindolin-5-yl)-2,5- diazabicyclo[2.2.1]heptan-2- yl)methyl)piperidin-1-yl)pyridin-2-yl)amino)- N,N-dimethyl-7H-pyrrolo[2,3-d]pyrimidine-6- carboxamide |
| | 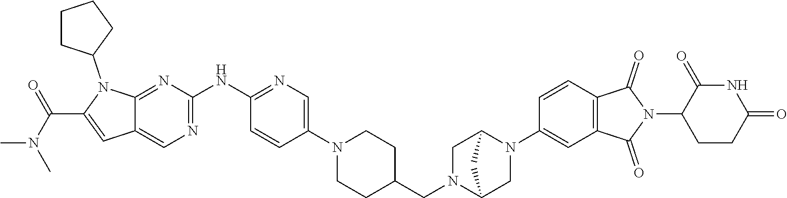 [SMILES: CN(C)C(=O)c1cc2cnc(Nc3ccc(N4CCC(CN5C[C@@H]6C[C@H]5CN6c5ccc6c(c5)C(=O)N(C5CCC(=O)NC5=O)C6=O)CC4)cn3)nc2n1C1CCCC1] | 7-cyclopentyl-2-((5-(4-(((1S,4S)-5-(2-(2,6- dioxopiperidin-3-yl)-1,3-dioxoisoindolin-5-yl)- 2,5-diazabicyclo [2.2.1]heptan-2- yl)methyl)piperidin-1-yl)pyridin-2-yl)amino)- N,N-dimethyl-7H-pyrrolo[2,3-d]pyrimidine-6- carboxamide |
| | 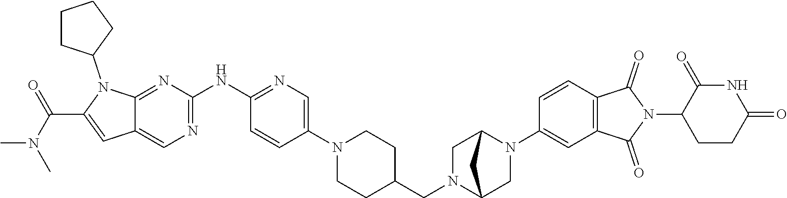 [SMILES: CN(C)C(=O)c1cc2cnc(Nc3ccc(N4CCC(CN5C[C@H]6C[C@@H]5CN6c5ccc6c(c5)C(=O)N(C5CCC(=O)NC5=O)C6=O)CC4)cn3)nc2n1C1CCCC1] | 7-cyclopentyl-2-((5-(4-(((1R,4R)-5-(2-(2,6- dioxopiperidin-3-yl)-1,3-dioxoisoindolin-5-yl)- 2,5-diazabicyclo[2.2.1]heptan-2- yl)methyl)piperidin-1-yl)pyridin-2-yl)amino)- N,N-dimethyl-7H-pyrrolo[2,3-d]pyrimidine-6- carboxamide |
| | 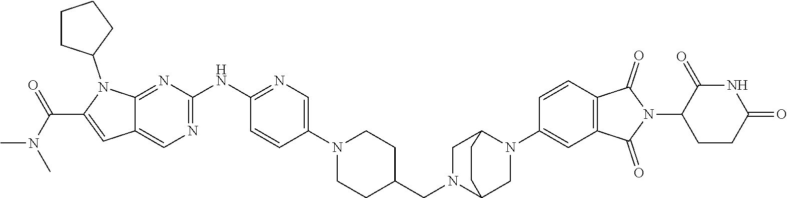 [SMILES: CN(C)C(=O)c1cc2cnc(Nc3ccc(N4CCC(CN5CC6CCC5CN6c5ccc6c(c5)C(=O)N(C5CCC(=O)NC5=O)C6=O)CC4)cn3)nc2n1C1CCCC1] | 7-cyclopentyl-2-((5-(4-((5-(2-(2,6- dioxopiperidin-3-yl)-1,3-dioxoisoindolin-5-yl)- 2,5-diazabicyclo[2.2.2]octan-2- yl)methyl)piperidin-1-yl)pyridin-2-yl)amino)- N,N-dimethyl-7H-pyrrolo[2,3-d]pyrimidine-6- carboxamide |
| | 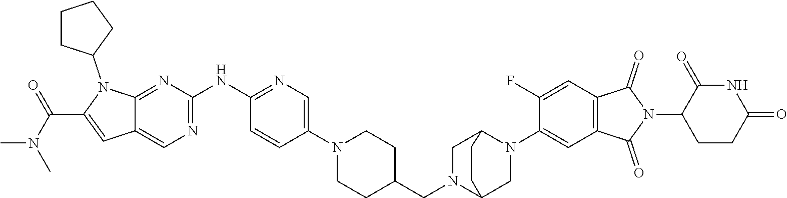 [SMILES: CN(C)C(=O)c1cc2cnc(Nc3ccc(N4CCC(CN5CC6CCC5CN6c5cc6c(cc5F)C(=O)N(C5CCC(=O)NC5=O)C6=O)CC4)cn3)nc2n1C1CCCC1] | 7-cyclopentyl-2-((5-(4-((5-(2-(2,6- dioxopiperidin-3-yl)-6-fluoro-1,3- dioxoisoindolin-5-yl)-2,5- diazabicyclo[2.2.2]octan-2-yl)methyl)piperidin- 1-yl)pyridin-2-yl)amino)-N,N-dimethyl-7H- pyrrolo[2,3-d]pyrimidine-6-carboxamide |
| | 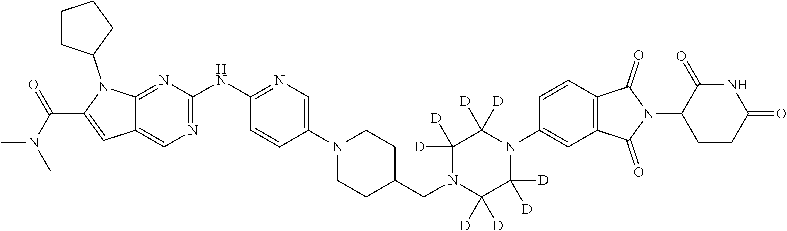 [SMILES: [2H]C1([2H])N(CC2CCN(c3ccc(Nc4ncc5cc(C(=O)N(C)C)n(C6CCCC6)c5n4)nc3)CC2)C([2H])([2H])C([2H])([2H])N(c2ccc3c(c2)C(=O)N(C2CCC(=O)NC2=O)C3=O)C1([2H])[2H]] | 7-cyclopentyl-2-((5-(4-((4-(2-(2,6- dioxopiperidin-3-yl)-1,3-dioxoisoindolin-5- yl)piperazin-1-yl-2,2,3,3,5,5,6,6- d₈)methyl)piperidin-1-yl)pyridin-2-yl)amino)- N,N-dimethyl-7H-pyrrolo[2,3-d]pyrimidine-6- carboxamide |
| | 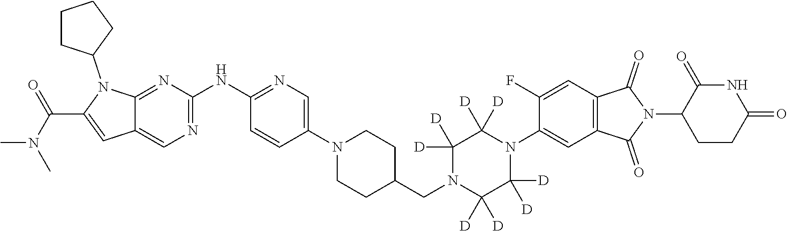 [SMILES: [2H]C1([2H])N(CC2CCN(c3ccc(Nc4ncc5cc(C(=O)N(C)C)n(C6CCCC6)c5n4)nc3)CC2)C([2H])([2H])C([2H])([2H])N(c2cc3c(cc2F)C(=O)N(C2CCC(=O)NC2=O)C3=O)C1([2H])[2H]] | 7-cyclopentyl-2-((5-(4-((4-(2-(2,6- dioxopiperidin-3-yl)-6-fluoro-1,3- dioxoisoindolin-5-yl)piperazin-1-yl- 2,2,3,3,5,5,6,6-d₈)methyl)piperidin-1- yl)pyridin-2-yl)amino)-N,N-dimethyl-7H- pyrrolo[2,3-d]pyrimidine-6-carboxamide |
| | 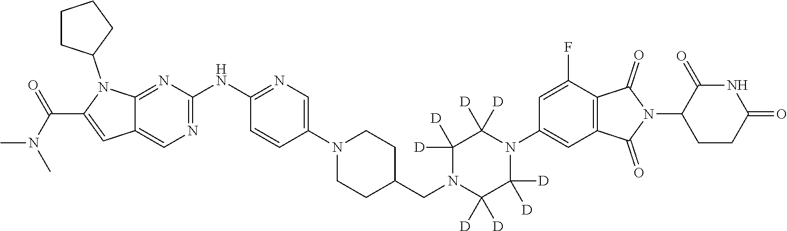 [SMILES: [2H]C1([2H])N(CC2CCN(c3ccc(Nc4ncc5cc(C(=O)N(C)C)n(C6CCCC6)c5n4)nc3)CC2)C([2H])([2H])C([2H])([2H])N(c2cc(F)c3c(c2)C(=O)N(C2CCC(=O)NC2=O)C3=O)C1([2H])[2H]] | 7-cyclopentyl-2-((5-(4-((4-(2-(2,6- dioxopiperidin-3-yl)-7-fluoro-1,3- dioxoisoindolin-5-yl)piperazin-1-yl- 2,2,3,3,5,5,6,6-d₈)methyl)piperidin-1- yl)pyridin-2-yl)amino)-N,N-dimethyl-7H- pyrrolo [2,3-d]pyrimidine-6-carboxamide |
| | 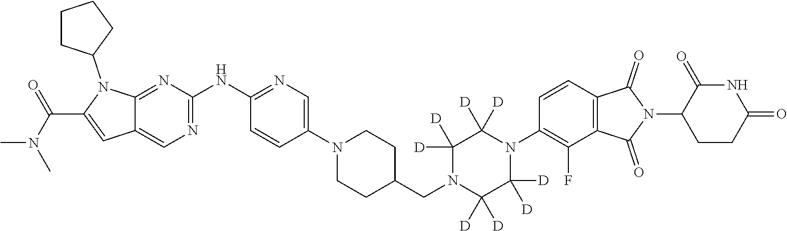 [SMILES: [2H]C1([2H])N(CC2CCN(c3ccc(Nc4ncc5cc(C(=O)N(C)C)n(C6CCCC6)c5n4)nc3)CC2)C([2H])([2H])C([2H])([2H])N(c2ccc3c(c2F)C(=O)N(C2CCC(=O)NC2=O)C3=O)C1([2H])[2H]] | 7-cyclopentyl-2-((5-(4-((4-(2-(2,6- dioxopiperidin-3-yl)-4-fluoro-1,3- dioxoisoindolin-5-yl)piperazin-1-yl- 2,2,3,3,5,5,6,6-d₈)methyl)piperidin-1- yl)pyridin-2-yl)amino)-N,N-dimethyl-7H- pyrrolo[2,3-d]pyrimidine-6-carboxamide |
| | 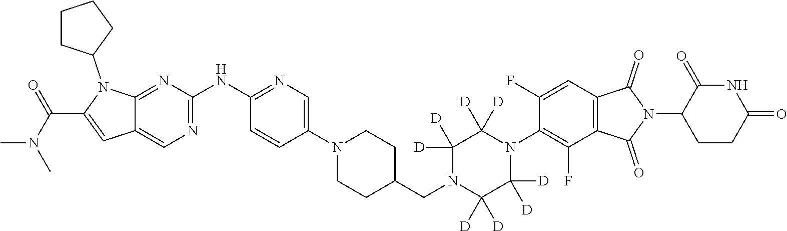 [SMILES: [2H]C1([2H])N(CC2CCN(c3ccc(Nc4ncc5cc(C(=O)N(C)C)n(C6CCCC6)c5n4)nc3)CC2)C([2H])([2H])C([2H])([2H])N(c2c(F)cc3c(c2F)C(=O)N(C2CCC(=O)NC2=O)C3=O)C1([2H])[2H]] | 7-cyclopentyl-2-((5-(4-((4-(2-(2,6- dioxopiperidin-3-yl)-4,6-difluoro-1,3- dioxoisoindolin-5-yl)piperazin-1-yl- 2,2,3,3,5,5,6,6-d₈)methyl)piperidin-1- yl)pyridin-2-yl)amino)-N,N-dimethyl-7H- pyrrolo[2,3-d]pyrimidine-6-carboxamide |
| | 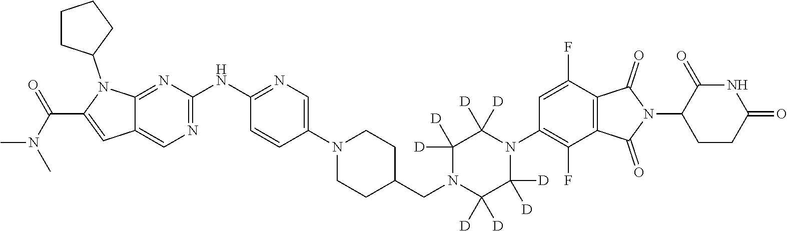 [SMILES: [2H]C1([2H])N(CC2CCN(c3ccc(Nc4ncc5cc(C(=O)N(C)C)n(C6CCCC6)c5n4)nc3)CC2)C([2H])([2H])C([2H])([2H])N(c2cc(F)c3c(c2F)C(=O)N(C2CCC(=O)NC2=O)C3=O)C1([2H])[2H]] | 7-cyclopentyl-2-((5-(4-((4-(2-(2,6- dioxopiperidin-3-yl)-4,7-difluoro-1,3- dioxoisoindolin-5-yl)piperazin-1-yl- 2,2,3,3,5,5,6,6-d₈)methyl)piperidin-1- yl)pyridin-2-yl)amino)-N,N-dimethyl-7H- pyrrolo[2,3-d]pyrimidine-6-carboxamide |
| | 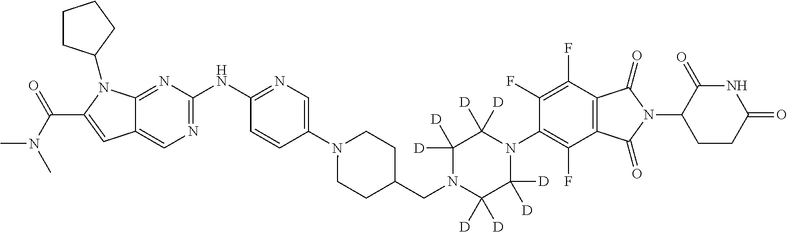 [SMILES: [2H]C1([2H])N(CC2CCN(c3ccc(Nc4ncc5cc(C(=O)N(C)C)n(C6CCCC6)c5n4)nc3)CC2)C([2H])([2H])C([2H])([2H])N(c2c(F)c(F)c3c(c2F)C(=O)N(C2CCC(=O)NC2=O)C3=O)C1([2H])[2H]] | 7-cyclopentyl-2-((5-(4-((4-(2-(2,6- dioxopiperidin-3-yl)-4,6,7-trifluoro-1,3- dioxoisoindolin-5-yl)piperazin-1-yl- 2,2,3,3,5,5,6,6-d₈)methyl)piperidin-1- yl)pyridin-2-yl)amino)-N,N-dimethyl-7H- pyrrolo[2,3-d]pyrimidine-6-carboxamide |
| | 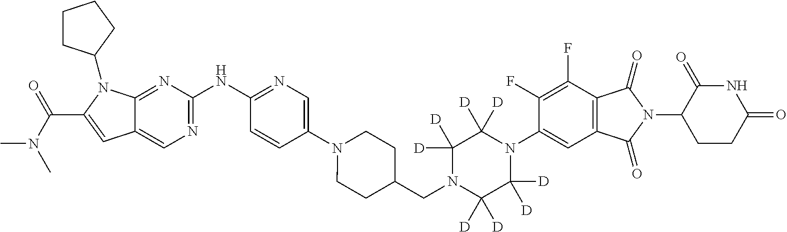 [SMILES: [2H]C1([2H])N(CC2CCN(c3ccc(Nc4ncc5cc(C(=O)N(C)C)n(C6CCCC6)c5n4)nc3)CC2)C([2H])([2H])C([2H])([2H])N(c2cc3c(c(F)c2F)C(=O)N(C2CCC(=O)NC2=O)C3=O)C1([2H])[2H]] | 7-cyclopentyl-2-((5-(4-((4-(2-(2,6- dioxopiperidin-3-yl)-6,7-difluoro-1,3- dioxoisoindolin-5-yl)piperazin-1-yl- 2,2,3,3,5,5,6,6-d₈)methyl)piperidin-1- yl)pyridin-2-yl)amino)-N,N-dimethyl-7H- pyrrolo[2,3-d]pyrimidine-6-carboxamide |
| | 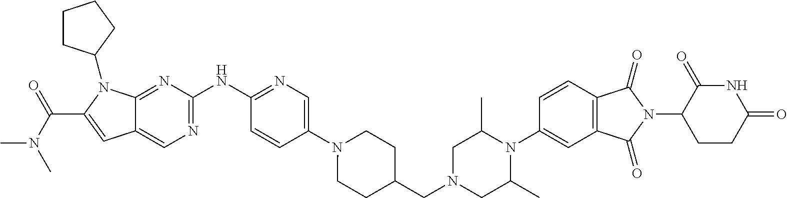 [SMILES: CC1CN(CC2CCN(c3ccc(Nc4ncc5cc(C(=O)N(C)C)n(C6CCCC6)c5n4)nc3)CC2)CC(C)N1c1ccc2c(c1)C(=O)N(C1CCC(=O)NC1=O)C2=O] | 7-cyclopentyl-2-((5-(4-((4-(2-(2,6- dioxopiperidin-3-yl)-1,3-dioxoisoindolin-5-yl)- 3,5-dimethylpiperazin-1-yl)methyl)piperidin-1- yl)pyridin-2-yl)amino)-N,N-dimethyl-7H- pyrrolo[2,3-d]pyrimidine-6-carboxamide |
| | 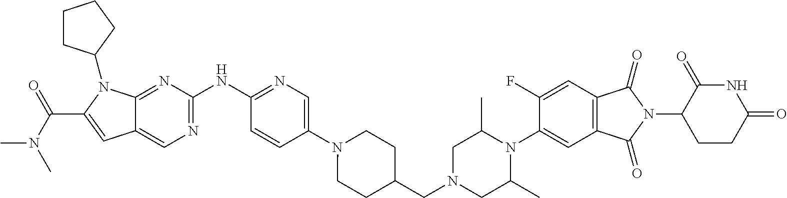 [SMILES: CC1CN(CC2CCN(c3ccc(Nc4ncc5cc(C(=O)N(C)C)n(C6CCCC6)c5n4)nc3)CC2)CC(C)N1c1cc2c(cc1F)C(=O)N(C1CCC(=O)NC1=O)C2=O] | 7-cyclopentyl-2-((5-(4-((4-(2-(2,6- dioxopiperidin-3-yl)-6-fluoro-1,3- dioxoisoindolin-5-yl)-3,5-dimethylpiperazin-1- yl)methyl)piperidin-1-yl)pyridin-2-yl)amino)- N,N-dimethyl-7H-pyrrolo[2,3-d]pyrimidine-6- carboxamide |
| | 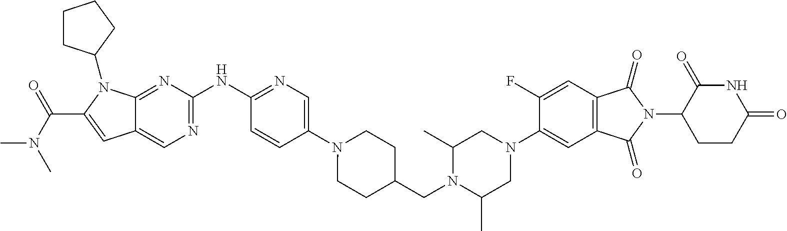 [SMILES: CC1CN(c2cc3c(cc2F)C(=O)N(C2CCC(=O)NC2=O)C3=O)CC(C)N1CC1CCN(c2ccc(Nc3ncc4cc(C(=O)N(C)C)n(C5CCCC5)c4n3)nc2)CC1] | 7-cyclopentyl-2-((5-(4-((4-(2-(2,6- dioxopiperidin-3-yl)-6-fluoro-1,3- dioxoisoindolin-5-yl)-2,6-dimethylpiperazin-1- yl)methyl)piperidin-1-yl)pyridin-2-yl)amino)- N,N-dimethyl-7H-pyrrolo[2,3-d]pyrimidine-6- carboxamide |
| | 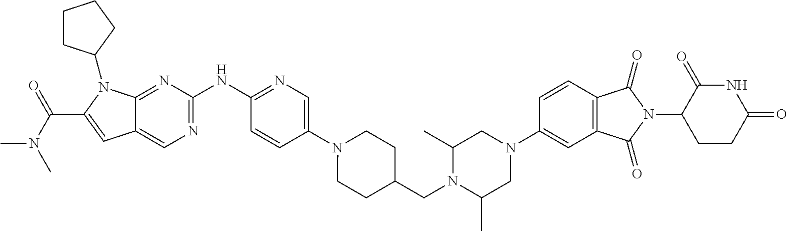 [SMILES: CC1CN(c2ccc3c(c2)C(=O)N(C2CCC(=O)NC2=O)C3=O)CC(C)N1CC1CCN(c2ccc(Nc3ncc4cc(C(=O)N(C)C)n(C5CCCC5)c4n3)nc2)CC1] | 7-cyclopentyl-2-((5-(4-((4-(2-(2,6- dioxopiperidin-3-yl)-1,3-dioxoisoindolin-5-yl)- 2,6-dimethylpiperazin-1-yl)methyl)piperidin-1- yl)pyridin-2-yl)amino)-N,N-dimethyl-7H- pyrrolo[2,3-d]pyrimidine-6-carboxamide |
| | 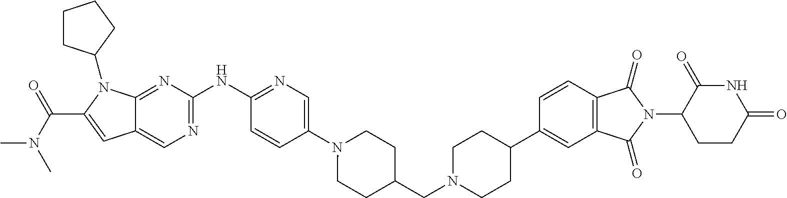 [SMILES: CN(C)C(=O)c1cc2cnc(Nc3ccc(N4CCC(CN5CCC(c6ccc7c(c6)C(=O)N(C6CCC(=O)NC6=O)C7=O)CC5)CC4)cn3)nc2n1C1CCCC1] | 7-cyclopentyl-2-((5-(4-((4-(2-(2,6- dioxopiperidin-3-yl)-1,3-dioxoisoindolin-5- yl)piperidin-1-yl)methyl)piperidin-1-yl)pyridin- 2-yl)amino)-N,N-dimethyl-7H-pyrrolo[2,3- d]pyrimidine-6-carboxamide |
| | 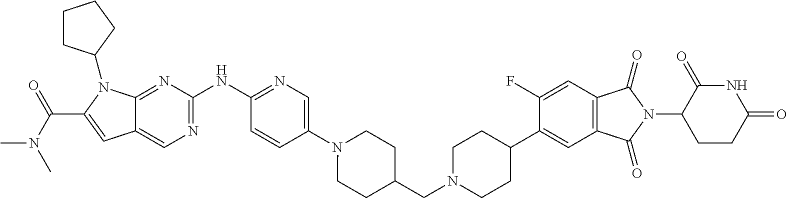 [SMILES: CN(C)C(=O)c1cc2cnc(Nc3ccc(N4CCC(CN5CCC(c6cc7c(cc6F)C(=O)N(C6CCC(=O)NC6=O)C7=O)CC5)CC4)cn3)nc2n1C1CCCC1] | 7-cyclopentyl-2-((5-(4-((4-(2-(2,6- dioxopiperidin-3-yl)-6-fluoro-1,3- dioxoisoindolin-5-yl)piperidin-1- yl)methyl)piperidin-1-yl)pyridin-2-yl)amino)- N,N-dimethyl-7H-pyrrolo[2,3-d]pyrimidine-6- carboxamide |
| | 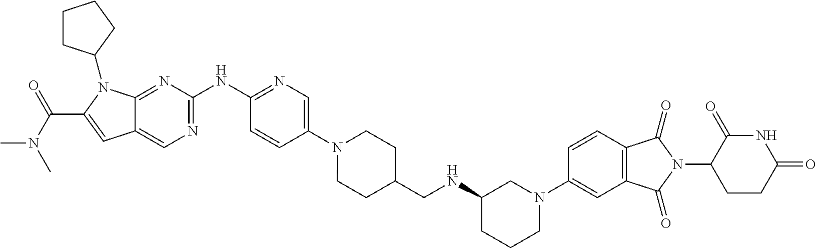 [SMILES: CN(C)C(=O)c1cc2cnc(Nc3ccc(N4CCC(CN[C@@H]5CCCN(c6ccc7c(c6)C(=O)N(C6CCC(=O)NC6=O)C7=O)C5)CC4)cn3)nc2n1C1CCCC1] | 7-cyclopentyl-2-((5-(4-((((3R)-1-(2-(2,6- dioxopiperidin-3-yl)-1,3-dioxoisoindolin-5- yl)piperidin-3-yl)amino)methyl)piperidin-1- yl)pyridin-2-yl)amino)-N,N-dimethyl-7H- pyrrolo[2,3-d]pyrimidine-6-carboxamide |
| | 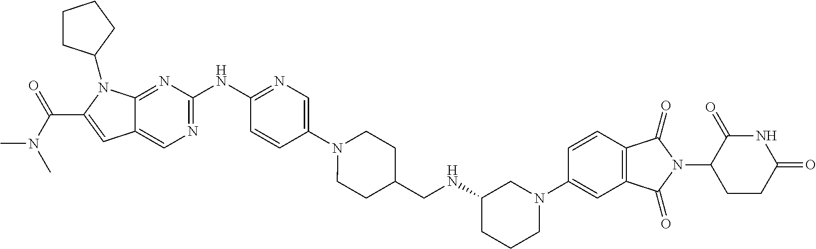 [SMILES: CN(C)C(=O)c1cc2cnc(Nc3ccc(N4CCC(CN[C@H]5CCCN(c6ccc7c(c6)C(=O)N(C6CCC(=O)NC6=O)C7=O)C5)CC4)cn3)nc2n1C1CCCC1] | 7-cyclopentyl-2-((5-(4-((((3S)-1-(2-(2,6- dioxopiperidin-3-yl)-1,3-dioxoisoindolin-5- yl)piperidin-3-yl)amino)methyl)piperidin-1- yl)pyridin-2-yl)amino)-N,N-dimethyl-7H- pyrrolo[2,3-d]pyrimidine-6-carboxamide |
| | 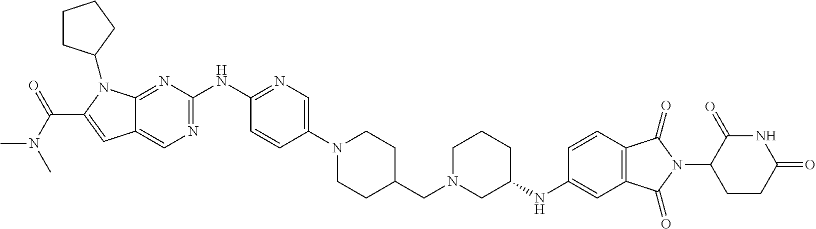 [SMILES: CN(C)C(=O)c1cc2cnc(Nc3ccc(N4CCC(CN5CCC[C@H](Nc6ccc7c(c6)C(=O)N(C6CCC(=O)NC6=O)C7=O)C5)CC4)cn3)nc2n1C1CCCC1] | 7-cyclopentyl-2-((5-(4-(((3S)-3-((2-(2,6- dioxopiperidin-3-yl)-1,3-dioxoisoindolin-5- yl)amino)piperidin-1-yl)methyl)piperidin-1- yl)pyridin-2-yl)amino)-N,N-dimethyl-7H- pyrrolo[2,3-d]pyrimidine-6-carboxamide |
| | 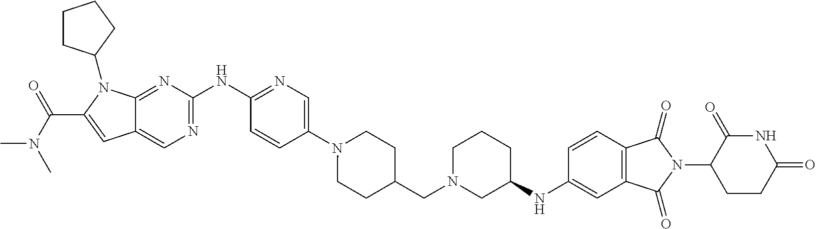 [SMILES: CN(C)C(=O)c1cc2cnc(Nc3ccc(N4CCC(CN5CCC[C@@H](Nc6ccc7c(c6)C(=O)N(C6CCC(=O)NC6=O)C7=O)C5)CC4)cn3)nc2n1C1CCCC1] | 7-cyclopentyl-2-((5-(4-(((3R)-3-((2-(2,6- dioxopiperidin-3-yl)-1,3-dioxoisoindolin-5- yl)amino)piperidin-1-yl)methyl)piperidin-1- yl)pyridin-2-yl)amino)-N,N-dimethyl-7H- pyrrolo[2,3-d]pyrimidine-6-carboxamide |
| | 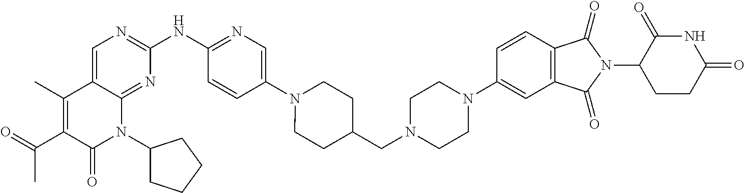 [SMILES: CC(=O)c1c(C)c2cnc(Nc3ccc(N4CCC(CN5CCN(c6ccc7c(c6)C(=O)N(C6CCC(=O)NC6=O)C7=O)CC5)CC4)cn3)nc2n(C2CCCC2)c1=O] | 5-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4- yl)methyl)piperazin-1-yl)-2-(2,6- dioxopiperidin-3-yl)isoindoline-1,3-dione |
| | 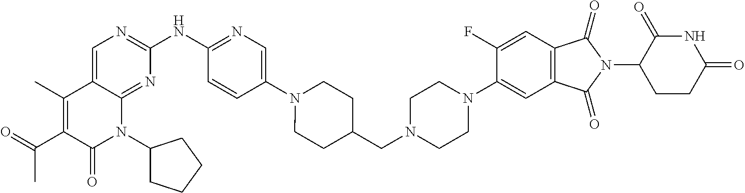 [SMILES: CC(=O)c1c(C)c2cnc(Nc3ccc(N4CCC(CN5CCN(c6cc7c(cc6F)C(=O)N(C6CCC(=O)NC6=O)C7=O)CC5)CC4)cn3)nc2n(C2CCCC2)c1=O] | 5-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4- yl)methyl)piperazin-1-yl)-2-(2,6- dioxopiperidin-3-yl)-6-fluoroisoindoline-1,3- dione |
| | 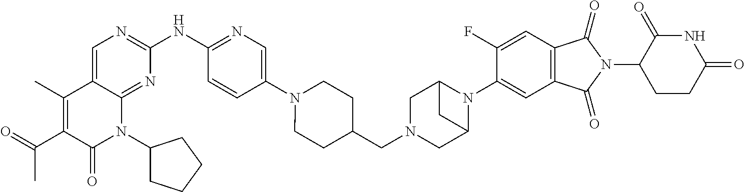 [SMILES: CC(=O)c1c(C)c2cnc(Nc3ccc(N4CCC(CN5CC6CC(C5)N6c5cc6c(cc5F)C(=O)N(C5CCC(=O)NC5=O)C6=O)CC4)cn3)nc2n(C2CCCC2)c1=O] | 5-(3-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 3,6-diazabicyclo[3.1.1]heptan-6-yl)-2-(2,6- dioxopiperidin-3-yl)-6-fluoroisoindoline-1,3- dione |
| | 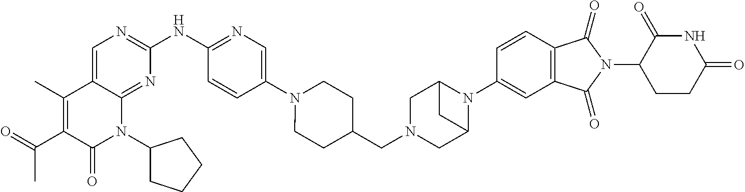 [SMILES: CC(=O)c1c(C)c2cnc(Nc3ccc(N4CCC(CN5CC6CC(C5)N6c5ccc6c(c5)C(=O)N(C5CCC(=O)NC5=O)C6=O)CC4)cn3)nc2n(C2CCCC2)c1=O] | 5-(3-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 3,6-diazabicyclo[3.1.1]heptan-6-yl)-2-(2,6- dioxopiperidin-3-yl)isoindoline-1,3-dione |
| | 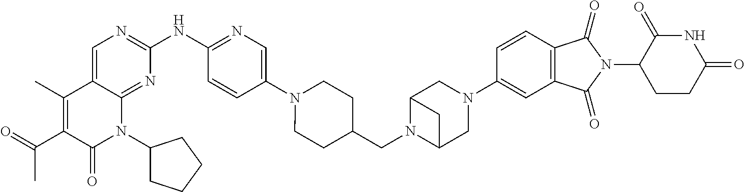 [SMILES: CC(=O)c1c(C)c2cnc(Nc3ccc(N4CCC(CN5C6CC5CN(c5ccc7c(c5)C(=O)N(C5CCC(=O)NC5=O)C7=O)C6)CC4)cn3)nc2n(C2CCCC2)c1=O] | 5-(6-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 3,6-diazabicyclo[3.1.1]heptan-3-yl)-2-(2,6- dioxopiperidin-3-yl)isoindoline-1,3-dione |
| | 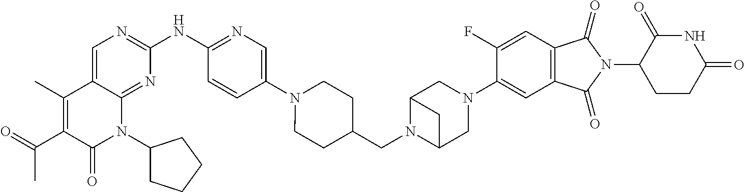 [SMILES: CC(=O)c1c(C)c2cnc(Nc3ccc(N4CCC(CN5C6CC5CN(c5cc7c(cc5F)C(=O)N(C5CCC(=O)NC5=O)C7=O)C6)CC4)cn3)nc2n(C2CCCC2)c1=O] | 5-(6-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 3,6-diazabicyclo[3.1.1]heptan-3-yl)-2-(2,6- dioxopiperidin-3-yl)-6-fluoroisoindoline-1,3- dione |
| | 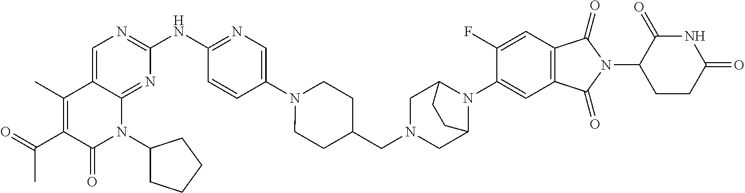 [SMILES: CC(=O)c1c(C)c2cnc(Nc3ccc(N4CCC(CN5CC6CCC(C5)N6c5cc6c(cc5F)C(=O)N(C5CCC(=O)NC5=O)C6=O)CC4)cn3)nc2n(C2CCCC2)c1=O] | 5-(3-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 3,8-diazabicyclo[3.2.1]octan-8-yl)-2-(2,6- dioxopiperidin-3-yl)-6-fluoroisoindoline-1,3- dione |
| | 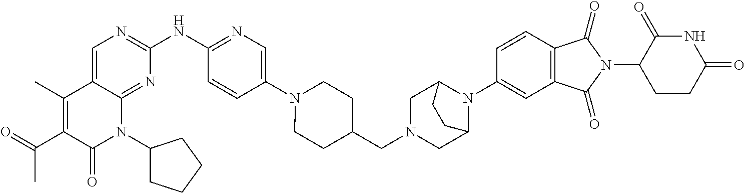 [SMILES: CC(=O)c1c(C)c2cnc(Nc3ccc(N4CCC(CN5CC6CCC(C5)N6c5ccc6c(c5)C(=O)N(C5CCC(=O)NC5=O)C6=O)CC4)cn3)nc2n(C2CCCC2)c1=O] | 5-(3-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 3,8-diazabicyclo[3.2.1]octan-8-yl)-2-(2,6- dioxopiperidin-3-yl)isoindoline-1,3-dione |
| | 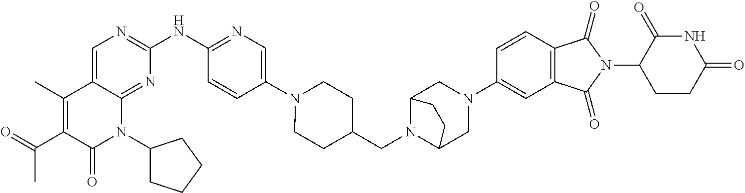 [SMILES: CC(=O)c1c(C)c2cnc(Nc3ccc(N4CCC(CN5C6CCC5CN(c5ccc7c(c5)C(=O)N(C5CCC(=O)NC5=O)C7=O)C6)CC4)cn3)nc2n(C2CCCC2)c1=O] | 5-(8-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 3,8-diazabicyclo[3.2.1]octan-3-yl)-2-(2,6- dioxopiperidin-3-yl)isoindoline-1,3-dione |
| | 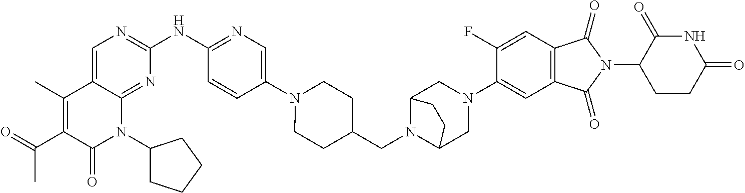 [SMILES: CC(=O)c1c(C)c2cnc(Nc3ccc(N4CCC(CN5C6CCC5CN(c5cc7c(cc5F)C(=O)N(C5CCC(=O)NC5=O)C7=O)C6)CC4)cn3)nc2n(C2CCCC2)c1=O] | 5-(8-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 3,8-diazabicyclo[3.2.1]octan-3-yl)-2-(2,6- dioxopiperidin-3-yl)-6-fluoroisoindoline-1,3- dione |
| | 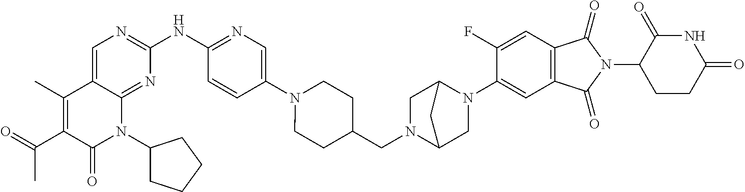 [SMILES: CC(=O)c1c(C)c2cnc(Nc3ccc(N4CCC(CN5CC6CC5CN6c5cc6c(cc5F)C(=O)N(C5CCC(=O)NC5=O)C6=O)CC4)cn3)nc2n(C2CCCC2)c1=O] | 5-(5-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 2,5-diazabicyclo[2.2.1]heptan-2-yl)-2-(2,6- dioxopiperidin-3-yl)-6-fluoroisoindoline-1,3- dione |
| | 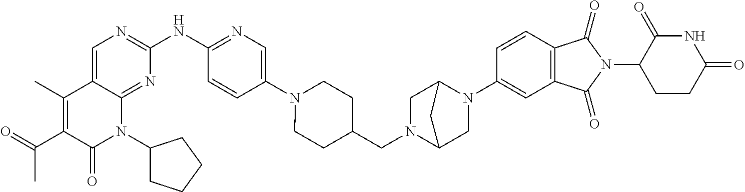 [SMILES: CC(=O)c1c(C)c2cnc(Nc3ccc(N4CCC(CN5CC6CC5CN6c5ccc6c(c5)C(=O)N(C5CCC(=O)NC5=O)C6=O)CC4)cn3)nc2n(C2CCCC2)c1=O] | 5-(5-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 2,5-diazabicyclo[2.2.1]heptan-2-yl)-2-(2,6- dioxopiperidin-3-yl)isoindoline-1,3-dione |
| | 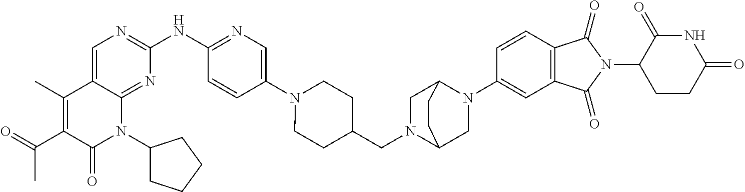 [SMILES: CC(=O)c1c(C)c2cnc(Nc3ccc(N4CCC(CN5CC6CCC5CN6c5ccc6c(c5)C(=O)N(C5CCC(=O)NC5=O)C6=O)CC4)cn3)nc2n(C2CCCC2)c1=O] | 5-(5-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 2,5-diazabicyclo[2.2.2]octan-2-yl)-2-(2,6- dioxopiperidin-3-yl)isoindoline-1,3-dione |
| | 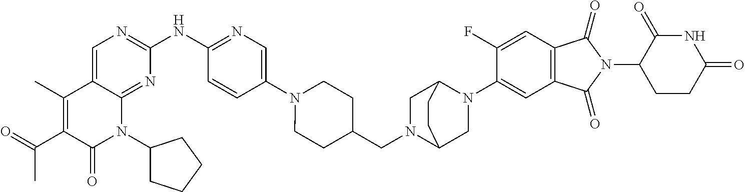 [SMILES: CC(=O)c1c(C)c2cnc(Nc3ccc(N4CCC(CN5CC6CCC5CN6c5cc6c(cc5F)C(=O)N(C5CCC(=O)NC5=O)C6=O)CC4)cn3)nc2n(C2CCCC2)c1=O] | 5-(5-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 2,5-diazabicyclo[2.2.2]octan-2-yl)-2-(2,6- dioxopiperidin-3-yl)-6-fluoroisoindoline-1,3- dione |
| | 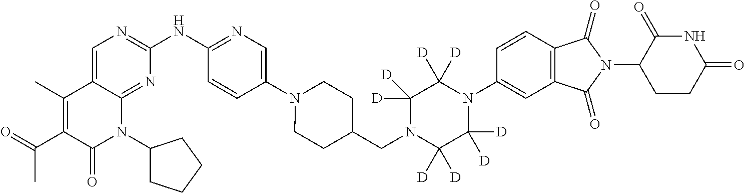 [SMILES: [2H]C1([2H])N(CC2CCN(c3ccc(Nc4ncc5c(C)c(C(C)=O)c(=O)n(C6CCCC6)c5n4)nc3)CC2)C([2H])([2H])C([2H])([2H])N(c2ccc3c(c2)C(=O)N(C2CCC(=O)NC2=O)C3=O)C1([2H])[2H]] | 5-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4- yl)methyl)piperazin-1-yl-2,2,3,3,5,5,6,6-d₈)-2- (2,6-dioxopiperidin-3-yl)isoindoline-1,3-dione |
| | 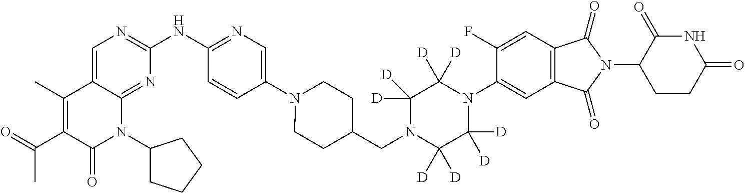 [SMILES: [2H]C1([2H])N(CC2CCN(c3ccc(Nc4ncc5c(C)c(C(C)=O)c(=O)n(C6CCCC6)c5n4)nc3)CC2)C([2H])([2H])C([2H])([2H])N(c2cc3c(cc2F)C(=O)N(C2CCC(=O)NC2=O)C3=O)C1([2H])[2H]] | 5-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4- yl)methyl)piperazin-1-yl-2,2,3,3,5,5,6,6-d₈)-2- (2,6-dioxopiperidin-3-yl)-6-fluoroisoindoline- 1,3-dione |
| | 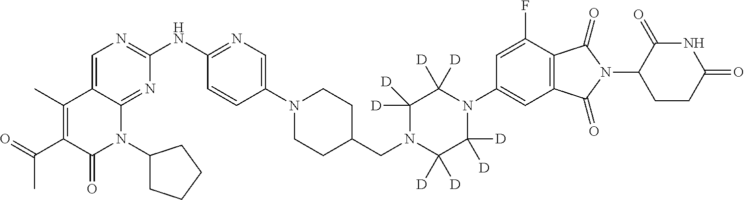 [SMILES: [2H]C1([2H])N(CC2CCN(c3ccc(Nc4ncc5c(C)c(C(C)=O)c(=O)n(C6CCCC6)c5n4)nc3)CC2)C([2H])([2H])C([2H])([2H])N(c2cc(F)c3c(c2)C(=O)N(C2CCC(=O)NC2=O)C3=O)C1([2H])[2H]] | 6-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 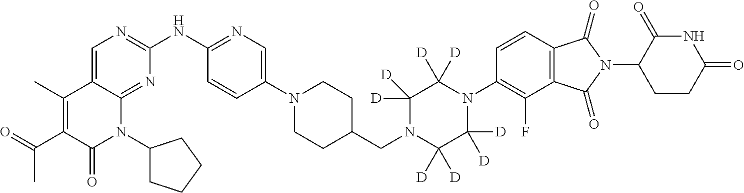 [SMILES: [2H]C1([2H])N(CC2CCN(c3ccc(Nc4ncc5c(C)c(C(C)=O)c(=O)n(C6CCCC6)c5n4)nc3)CC2)C([2H])([2H])C([2H])([2H])N(c2ccc3c(c2F)C(=O)N(C2CCC(=O)NC2=O)C3=O)C1([2H])[2H]] | 5-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 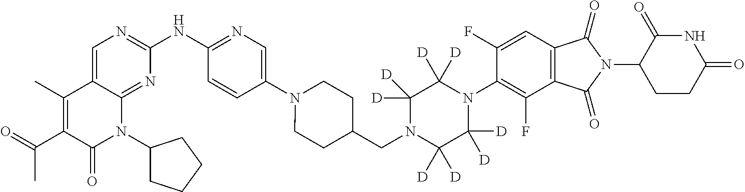 [SMILES: [2H]C1([2H])N(CC2CCN(c3ccc(Nc4ncc5c(C)c(C(C)=O)c(=O)n(C6CCCC6)c5n4)nc3)CC2)C([2H])([2H])C([2H])([2H])N(c2c(F)cc3c(c2F)C(=O)N(C2CCC(=O)NC2=O)C3=O)C1([2H])[2H]] | 5-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4- yl)methyl)piperazin-1-yl-2,2,3,3,5,5,6,6-d₈)-2- (2,6-dioxopiperidin-3-yl)-4,6- difluoroisoindoline-1,3-dione |
| | 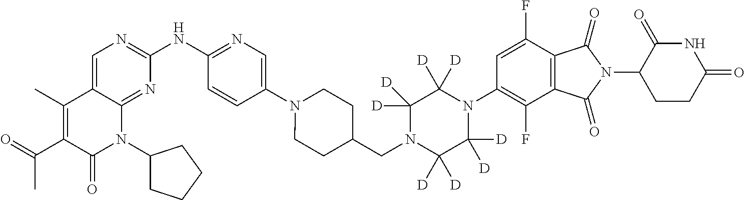 [SMILES: [2H]C1([2H])N(CC2CCN(c3ccc(Nc4ncc5c(C)c(C(C)=O)c(=O)n(C6CCCC6)c5n4)nc3)CC2)C([2H])([2H])C([2H])([2H])N(c2cc(F)c3c(c2F)C(=O)N(C2CCC(=O)NC2=O)C3=O)C1([2H])[2H]] | 5-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4- yl)methyl)piperazin-1-yl-2,2,3,3,5,5,6,6-d₈)-2- (2,6-dioxopiperidin-3-yl)-4,7- difluoroisoindoline-1,3-dione |
| | 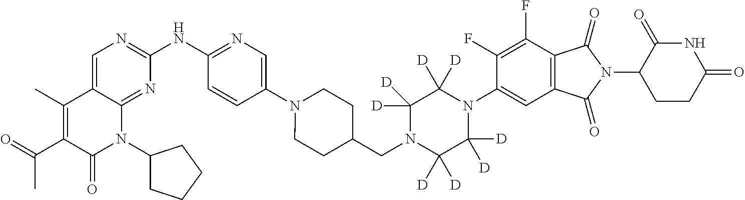 [SMILES: [2H]C1([2H])N(CC2CCN(c3ccc(Nc4ncc5c(C)c(C(C)=O)c(=O)n(C6CCCC6)c5n4)nc3)CC2)C([2H])([2H])C([2H])([2H])N(c2cc3c(c(F)c2F)C(=O)N(C2CCC(=O)NC2=O)C3=O)C1([2H])[2H]] | 6-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4- yl)methyl)piperazin-1-yl-2,2,3,3,5,5,6,6-d₈)-2- (2,6-dioxopiperidin-3-yl)-4,5- difluoroisoindoline-1,3-dione |
| | 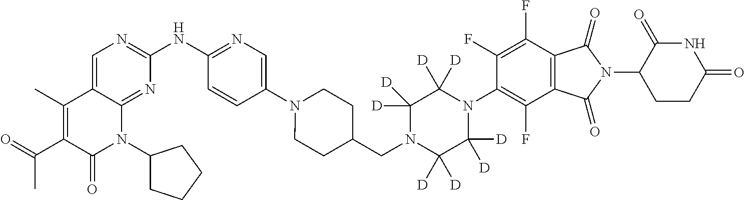 [SMILES: [2H]C1([2H])N(CC2CCN(c3ccc(Nc4ncc5c(C)c(C(C)=O)c(=O)n(C6CCCC6)c5n4)nc3)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4- yl)methyl)piperazin-1-yl-2,2,3,3,5,5,6,6-d₈)-2- (2,6-dioxopiperidin-3-yl)-4,6,7- trifluoroisoindoline-1,3-dione |
| | 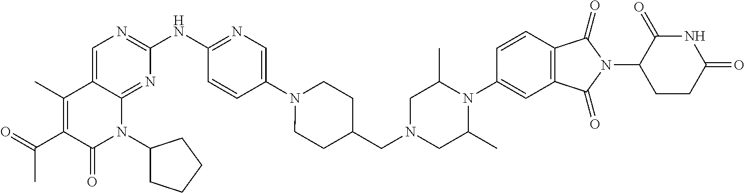 [SMILES: CC(=O)c1c(C)c2cnc(Nc3ccc(N4CCC(CN5CC(C)N(c6ccc7c(c6)C(=O)N(C6CCC(=O)NC6=O)C7=O)C(C)C5)CC4)cn3)nc2n(C2CCCC2)c1=O] | 5-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 2,6-dimethylpiperazin-1-yl)-2-(2,6- dioxopiperidin-3-yl)isoindoline-1,3-dione |
| | 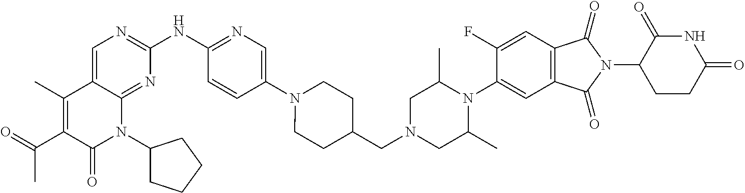 [SMILES: CC(=O)c1c(C)c2cnc(Nc3ccc(N4CCC(CN5CC(C)N(c6cc7c(cc6F)C(=O)N(C6CCC(=O)NC6=O)C7=O)C(C)C5)CC4)cn3)nc2n(C2CCCC2)c1=O] | 5-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 2,6-dimethylpiperazin-1-yl)-2-(2,6- dioxopiperidin-3-yl)-6-fluoroisoindoline-1,3- dione |
| | 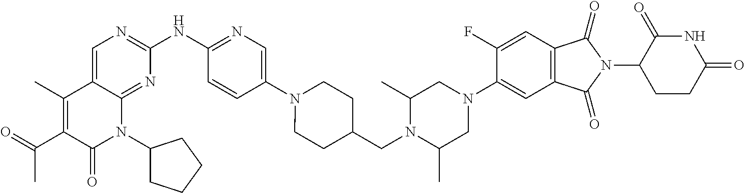 [SMILES: CC(=O)c1c(C)c2cnc(Nc3ccc(N4CCC(CN5C(C)CN(c6cc7c(cc6F)C(=O)N(C6CCC(=O)NC6=O)C7=O)CC5C)CC4)cn3)nc2n(C2CCCC2)c1=O] | 5-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 3,5-dimethylpiperazin-1-yl)-2-(2,6- dioxopiperidin-3-yl)-6-fluoroisoindoline-1,3- dione |
| | 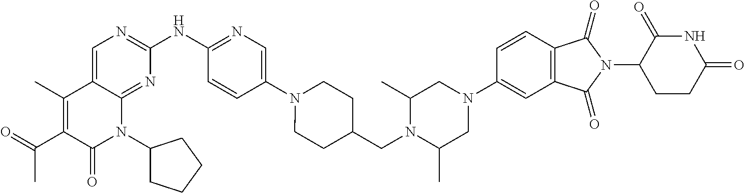 [SMILES: CC(=O)c1c(C)c2cnc(Nc3ccc(N4CCC(CN5C(C)CN(c6ccc7c(c6)C(=O)N(C6CCC(=O)NC6=O)C7=O)CC5C)CC4)cn3)nc2n(C2CCCC2)c1=O] | 5-(4-((1-(6-((6-acetyl-8-cyclopentyl-5-methyl- 7-oxo-7,8-dihydropyrido[2,3-d]pyrimidin-2- yl)amino)pyridin-3-yl)piperidin-4-yl)methyl)- 3,5-dimethylpiperazin-1-yl)-2-(2,6- dioxopiperidin-3-yl)isoindoline-1,3-dione |
| | 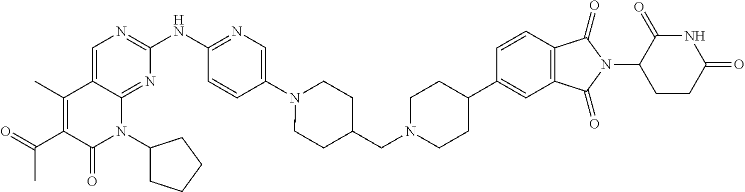 [SMILES: CC(=O)c1c(C)c2cnc(Nc3ccc(N4CCC(CN5CCC(c6ccc7c(c6)C(=O)N(C6CCC(=O)NC6=O)C7=O)CC5)CC4)cn3)nc2n(C2CCCC2)c1=O] | 5-(1-((1-(6-((6-acetyl-8-cyclopentyl-5-methyl- 7-oxo-7,8-dihydropyrido[2,3-d]pyrimidin-2- yl)amino)pyridin-3-yl)piperidin-4- yl)methyl)piperidin-4-yl)-2-(2,6- dioxopiperidin-3-yl)isoindoline-1,3-dione |
| | 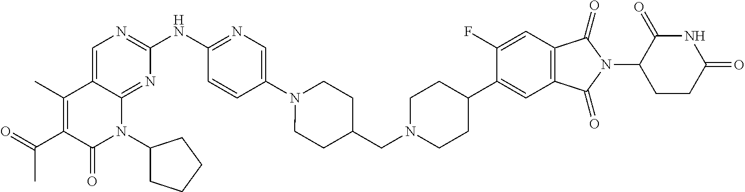 [SMILES: CC(=O)c1c(C)c2cnc(Nc3ccc(N4CCC(CN5CCC(c6cc7c(cc6F)C(=O)N(C6CCC(=O)NC6=O)C7=O)CC5)CC4)cn3)nc2n(C2CCCC2)c1=O] | 5-(1-((1-(6-((6-acetyl-8-cyclopentyl-5-methyl- 7-oxo-7,8-dihydropyrido[2,3-d]pyrimidin-2- yl)amino)pyridin-3-yl)piperidin-4- yl)methyl)piperidin-4-yl)-2-(2,6- dioxopiperidin-3-yl)-6-fluoroisoindoline-1,3- dione |
| | 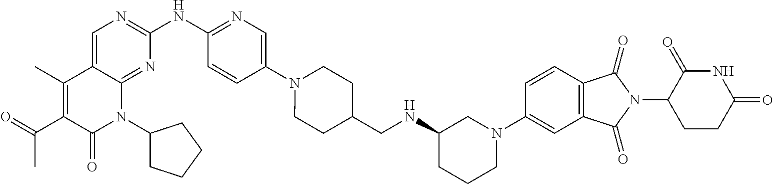 [SMILES: CC(=O)c1c(C)c2cnc(Nc3ccc(N4CCC(CN[C@@H]5CCCN(c6ccc7c(c6)C(=O)N(C6CCC(=O)NC6=O)C7=O)C5)CC4)cn3)nc2n(C2CCCC2)c1=O] | 5-((R)-3-(((1-(6-((6-acetyl-8-cyclopentyl-5- methyl-7-oxo-7,8-dihydropyrido[2,3- d]pyrimidin-2-yl)amino)pyridin-3-yl)piperidin- 4-yl)methyl)amino)piperidin-1-yl)-2-(2,6- dioxopiperidin-3-yl)isoindoline-1,3-dione |
| | 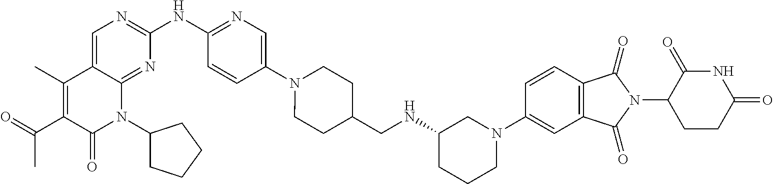 [SMILES: CC(=O)c1c(C)c2cnc(Nc3ccc(N4CCC(CN[C@H]5CCCN(c6ccc7c(c6)C(=O)N(C6CCC(=O)NC6=O)C7=O)C5)CC4)cn3)nc2n(C2CCCC2)c1=O] | 5-((S)-3-(((1-(6-((6-acetyl-8-cyclopentyl-5- methyl-7-oxo-7,8-dihydropyrido[2,3- d]pyrimidin-2-yl)amino)pyridin-3-yl)piperidin- 4-yl)methyl)amino)piperidin-1-yl)-2-(2,6- dioxopiperidin-3-yl)isoindoline-1,3-dione |
| | 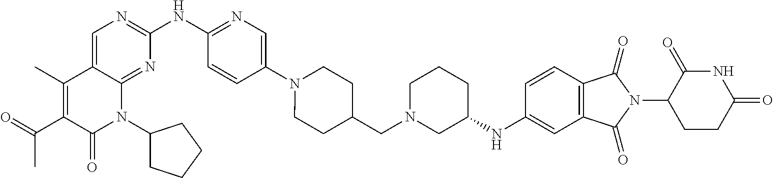 [SMILES: CC(=O)c1c(C)c2cnc(Nc3ccc(N4CCC(CN5CCC[C@H](Nc6ccc7c(c6)C(=O)N(C6CCC(=O)NC6=O)C7=O)C5)CC4)cn3)nc2n(C2CCCC2)c1=O] | 5-(((S)-1-((1-(6-((6-acetyl-8-cyclopentyl-5- methyl-7-oxo-7,8-dihydropyrido[2,3- d]pyrimidin-2-yl)amino)pyridin-3-yl)piperidin- 4-yl)methyl)piperidin-3-yl)amino)-2-(2,6- dioxopiperidin-3-yl)isoindoline-1,3-dione |
| | 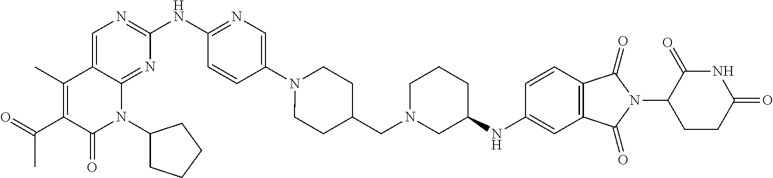 [SMILES: CC(=O)c1c(C)c2cnc(Nc3ccc(N4CCC(CN5CCC[C@@H](Nc6ccc7c(c6)C(=O)N(C6CCC(=O)NC6=O)C7=O)C5)CC4)cn3)nc2n(C2CCCC2)c1=O] | 5-(((R)-1-((1-(6-((6-acetyl-8-cyclopentyl-5- methyl-7-oxo-7,8-dihydropyrido[2,3- d]pyrimidin-2-yl)amino)pyridin-3-yl)piperidin- 4-yl)methyl)piperidin-3-yl)amino)-2-(2,6- dioxopiperidin-3-yl)isoindoline-1,3-dione |
| | 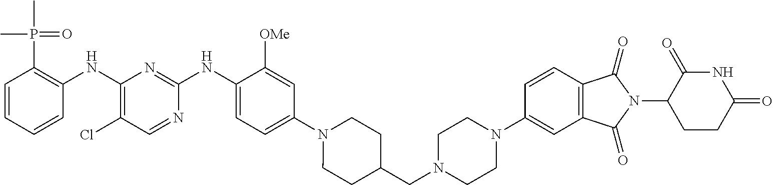 [SMILES: COc1cc(N2CCC(CN3CCN(c4ccc5c(c4)C(=O)N(C4CCC(=O)NC4=O)C5=O)CC3)CC2)ccc1Nc1ncc(Cl)c(Nc2ccccc2P(C)(C)=O)n1] | 5-(4-((1-(4-((5-chloro-4-((2- (dimethylphosphoryl)phenyl)amino)pyrimidin- 2-yl)amino)-3-methoxyphenyl)piperidin-4- yl)methyl)piperazin-1-yl)-2-(2,6- dioxopiperidin-3-yl)isoindoline-1,3-dione |
| | 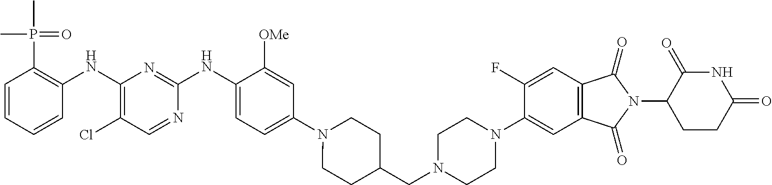 [SMILES: COc1cc(N2CCC(CN3CCN(c4cc5c(cc4F)C(=O)N(C4CCC(=O)NC4=O)C5=O)CC3)CC2)ccc1Nc1ncc(Cl)c(Nc2ccccc2P(C)(C)=O)n1] | 5-(4-((1-(4-((5-chloro-4-((2- (dimethylphosphoryl)phenyl)amino)pyrimidin- 2-yl)amino)-3-methoxyphenyl)piperidin-4- yl)methyl)piperazin-1-yl)-2-(2,6- dioxopiperidin-3-yl)-6-fluoroisoindoline-1,3- dione |
| | 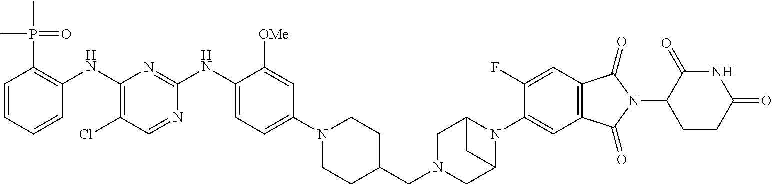 [SMILES: COc1cc(N2CCC(CN3CC4CC(C3)N4c3cc4c(cc3F)C(=O)N(C3CCC(=O)NC3=O)C4=O)CC2)ccc1Nc1ncc(Cl)c(Nc2ccccc2P(C)(C)=O)n1] | 5-(3-((1-(4-((5-chloro-4-((2- (dimethylphosphoryl)phenyl)amino)pyrimidin- 2-yl)amino)-3-methoxyphenyl)piperidin-4- yl)methyl)-3,6-diazabicyclo[3.1.1]heptan-6-yl)- 2-(2,6-dioxopiperidin-3-yl)-6- fluoroisoindoline-1,3-dione |
| | 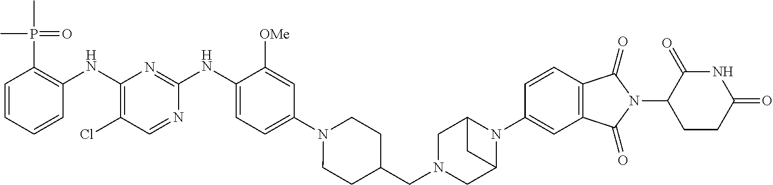 [SMILES: COc1cc(N2CCC(CN3CC4CC(C3)N4c3ccc4c(c3)C(=O)N(C3CCC(=O)NC3=O)C4=O)CC2)ccc1Nc1ncc(Cl)c(Nc2ccccc2P(C)(C)=O)n1] | 5-(3-((1-(4-((5-chloro-4-((2- (dimethylphosphoryl)phenyl)amino)pyrimidin- 2-yl)amino)-3-methoxyphenyl)piperidin-4- yl)methyl)-3,6-diazabicyclo[3.1.1]heptan-6-yl)- 2-(2,6-dioxopiperidin-3-yl)isoindoline-1,3- dione |
| | 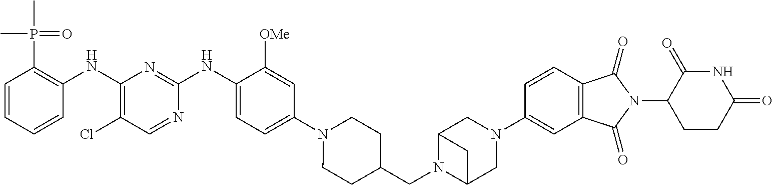 [SMILES: COc1cc(N2CCC(CN3C4CC3CN(c3ccc5c(c3)C(=O)N(C3CCC(=O)NC3=O)C5=O)C4)CC2)ccc1Nc1ncc(Cl)c(Nc2ccccc2P(C)(C)=O)n1] | 5-(6-((1-(4-((5-chloro-4-((2- (dimethylphosphoryl)phenyl)amino)pyrimidin- 2-yl)amino)-3-methoxyphenyl)piperidin-4- yl)methyl)-3,6-diazabicyclo[3.1.1]heptan-3-yl)- 2-(2,6-dioxopiperidin-3-yl)isoindoline-1,3- dione |
| | 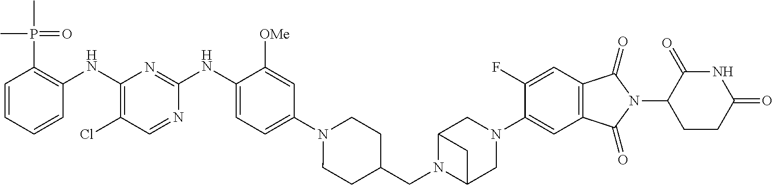 [SMILES: COc1cc(N2CCC(CN3C4CC3CN(c3cc5c(cc3F)C(=O)N(C3CCC(=O)NC3=O)C5=O)C4)CC2)ccc1Nc1ncc(Cl)c(Nc2ccccc2P(C)(C)=O)n1] | 5-(6-((1-(4-((5-chloro-4-((2- (dimethylphosphoryl)phenyl)amino)pyrimidin- 2-yl)amino)-3-methoxyphenyl)piperidin-4- yl)methyl)-3,6-diazabicyclo[3.1.1]heptan-3-yl)- 2-(2,6-dioxopiperidin-3-yl)-6- fluoroisoindoline-1,3-dione |
| | 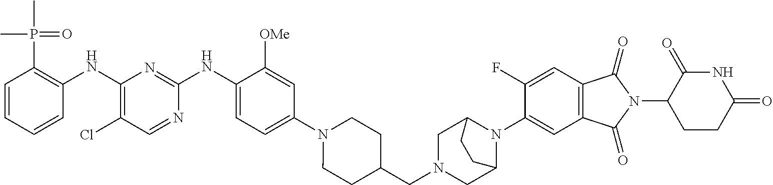 [SMILES: COc1cc(N2CCC(CN3CC4CCC(C3)N4c3cc4c(cc3F)C(=O)N(C3CCC(=O)NC3=O)C4=O)CC2)ccc1Nc1ncc(Cl)c(Nc2ccccc2P(C)(C)=O)n1] | 5-(3-((1-(4-((5-chloro-4-((2- (dimethylphosphoryl)phenyl)amino)pyrimidin- 2-yl)amino)-3-methoxyphenyl)piperidin-4- yl)methyl)-3,8-diazabicyclo[3.2.1]octan-8-yl)- 2-(2,6-dioxopiperidin-3-yl)-6- fluoroisoindoline-1,3-dione |
| | 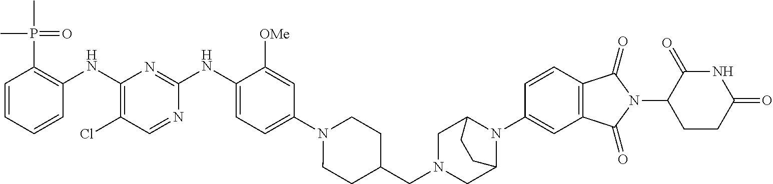 [SMILES: COc1cc(N2CCC(CN3CC4CCC(C3)N4c3ccc4c(c3)C(=O)N(C3CCC(=O)NC3=O)C4=O)CC2)ccc1Nc1ncc(Cl)c(Nc2ccccc2P(C)(C)=O)n1] | 5-(3-((1-(4-((5-chloro-4-((2- (dimethylphosphoryl)phenyl)amino)pyrimidin- 2-yl)amino)-3-methoxyphenyl)piperidin-4- yl)methyl)-3,8-diazabicyclo[3.2.1]octan-8-yl)- 2-(2,6-dioxopiperidin-3-yl)isoindoline-1,3- dione |
| | 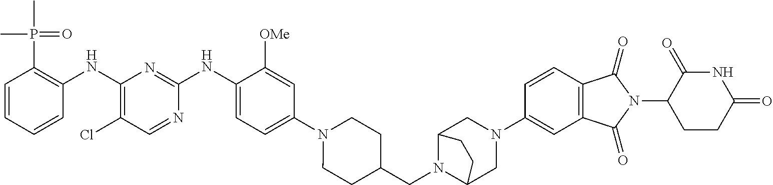 [SMILES: COc1cc(N2CCC(CN3C4CCC3CN(c3ccc5c(c3)C(=O)N(C3CCC(=O)NC3=O)C5=O)C4)CC2)ccc1Nc1ncc(Cl)c(Nc2ccccc2P(C)(C)=O)n1] | 5-(8-((1-(4-((5-chloro-4-((2- (dimethylphosphoryl)phenyl)amino)pyrimidin- 2-yl)amino)-3-methoxyphenyl)piperidin-4- yl)methyl)-3,8-diazabicyclo[3.2.1]octan-3-yl)- 2-(2,6-dioxopiperidin-3-yl)isoindoline-1,3- dione |
| | 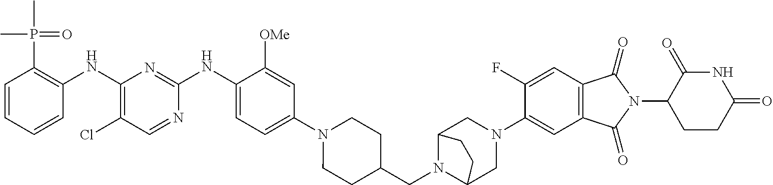 [SMILES: COc1cc(N2CCC(CN3C4CCC3CN(c3cc5c(cc3F)C(=O)N(C3CCC(=O)NC3=O)C5=O)C4)CC2)ccc1Nc1ncc(Cl)c(Nc2ccccc2P(C)(C)=O)n1] | 5-(8-((1-(4-((5-chloro-4-((2- (dimethylphosphoryl)phenyl)amino)pyrimidin- 2-yl)amino)-3-methoxyphenyl)piperidin-4- yl)methyl)-3,8-diazabicyclo[3.2.1]octan-3-yl)- 2-(2,6-dioxopiperidin-3-yl)-6- fluoroisoindoline-1,3-dione |
| | 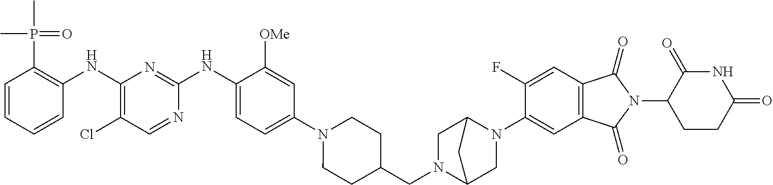 [SMILES: COc1cc(N2CCC(CN3CC4CC3CN4c3cc4c(cc3F)C(=O)N(C3CCC(=O)NC3=O)C4=O)CC2)ccc1Nc1ncc(Cl)c(Nc2ccccc2P(C)(C)=O)n1] | 5-(5-((1-(4-((5-chloro-4-((2- (dimethylphosphoryl)phenyl)amino)pyrimidin- 2-yl)amino)-3-methoxyphenyl)piperidin-4- yl)methyl)-2,5-diazabicyclo[2.2.1]heptan-2-yl)- 2-(2,6-dioxopiperidin-3-yl)-6- fluoroisoindoline-1,3-dione |
| | 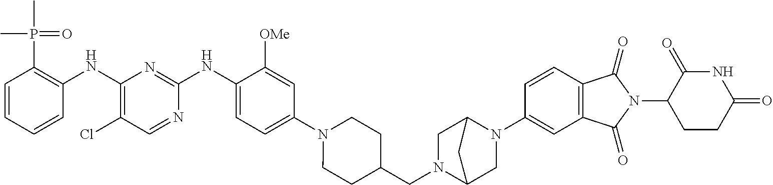 [SMILES: COc1cc(N2CCC(CN3CC4CC3CN4c3ccc4c(c3)C(=O)N(C3CCC(=O)NC3=O)C4=O)CC2)ccc1Nc1ncc(Cl)c(Nc2ccccc2P(C)(C)=O)n1] | 5-(5-((1-(4-((5-chloro-4-((2- (dimethylphosphoryl)phenyl)amino)pyrimidin- 2-yl)amino)-3-methoxyphenyl)piperidin-4- yl)methyl)-2,5-diazabicyclo[2.2.1]heptan-2-yl)- 2-(2,6-dioxopiperidin-3-yl)isoindoline-1,3- dione |
| | 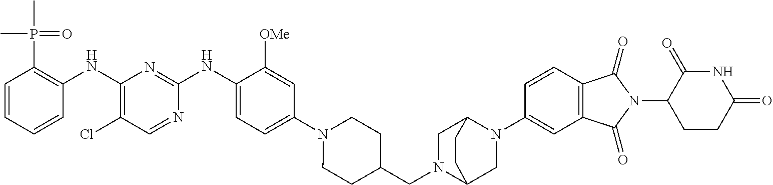 [SMILES: COc1cc(N2CCC(CN3CC4CCC3CN4c3ccc4c(c3)C(=O)N(C3CCC(=O)NC3=O)C4=O)CC2)ccc1Nc1ncc(Cl)c(Nc2ccccc2P(C)(C)=O)n1] | 5-(5-((1-(4-((5-chloro-4-((2- (dimethylphosphoryl)phenyl)amino)pyrimidin- 2-yl)amino)-3-methoxyphenyl)piperidin-4- yl)methyl)-2,5-diazabicyclo[2.2.2]octan-2-yl)- 2-(2,6-dioxopiperidin-3-yl)isoindoline-1,3- dione |
| | 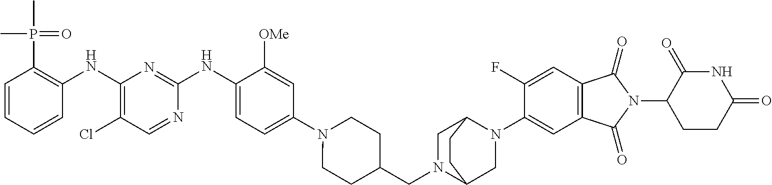 [SMILES: COc1cc(N2CCC(CN3CC4CCC3CN4c3cc4c(cc3F)C(=O)N(C3CCC(=O)NC3=O)C4=O)CC2)ccc1Nc1ncc(Cl)c(Nc2ccccc2P(C)(C)=O)n1] | 5-(5-((1-(4-((5-chloro-4-((2- (dimethylphosphoryl)phenyl)amino)pyrimidin- 2-yl)amino)-3-methoxyphenyl)piperidin-4- yl)methyl)-2,5-diazabicyclo[2.2.2]octan-2-yl)- 2-(2,6-dioxopiperidin-3-yl)-6- fluoroisoindoline-1,3-dione |
| | 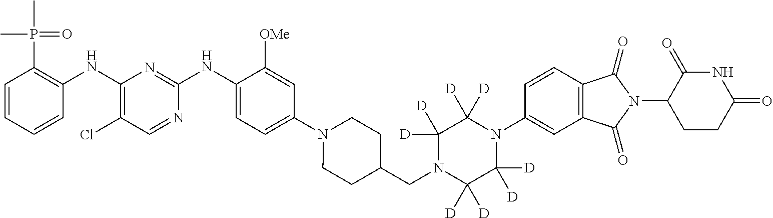 [SMILES: [2H]C1([2H])N(CC2CCN(c3ccc(Nc4ncc(Cl)c(Nc5ccccc5P(C)(C)=O)n4)c(OC)c3)CC2)C([2H])([2H])C([2H])([2H])N(c2ccc3c(c2)C(=O)N(C2CCC(=O)NC2=O)C3=O)C1([2H])[2H]] | 5-(4-((1-(4-((5-chloro-4-((2- (dimethylphosphoryl)phenyl)amino)pyrimidin- 2-yl)amino)-3-methoxyphenyl)piperidin-4- yl)methyl)piperazin-1-yl-2,2,3,3,5,5,6,6-d₈)-2- (2,6-dioxopiperidin-3-yl)isoindoline-1,3-dione |
| | 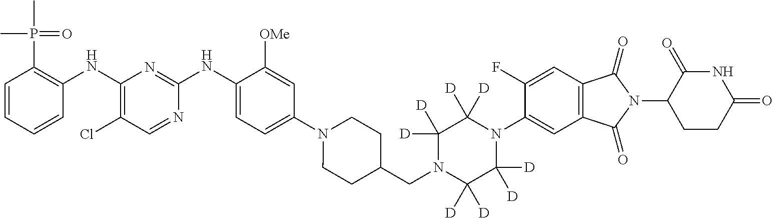 [SMILES: [2H]C1([2H])N(CC2CCN(c3ccc(Nc4ncc(Cl)c(Nc5ccccc5P(C)(C)=O)n4)c(OC)c3)CC2)C([2H])([2H])C([2H])([2H])N(c2cc3c(cc2F)C(=O)N(C2CCC(=O)NC2=O)C3=O)C1([2H])[2H]] | 5-(4-((1-(4-((5-chloro-4-((2- (dimethylphosphoryl)phenyl)amino)pyrimidin- 2-yl)amino)-3-methoxyphenyl)piperidin-4- yl)methyl)piperazin-1-yl-2,2,3,3,5,5,6,6-d₈)-2- (2,6-dioxopiperidin-3-yl)-6-fluoroisoindoline- 1,3-dione |
| | 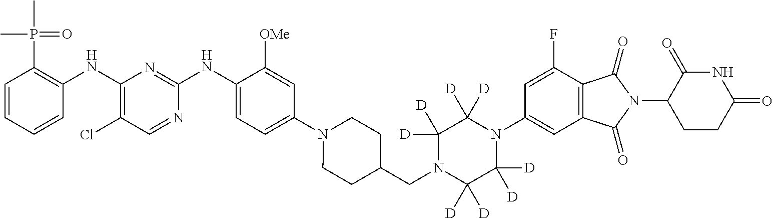 [SMILES: [2H]C1([2H])N(CC2CCN(c3ccc(Nc4ncc(Cl)c(Nc5ccccc5P(C)(C)=O)n4)c(OC)c3)CC2)C([2H])([2H])C([2H])([2H])N(c2cc(F)c3c(c2)C(=O)N(C2CCC(=O)NC2=O)C3=O)C1([2H])[2H]] | 6-(4-((1-(4-((5-chloro-4-((2- (dimethylphosphoryl)phenyl)amino)pyrimidin- 2-yl)amino)-3-methoxyphenyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 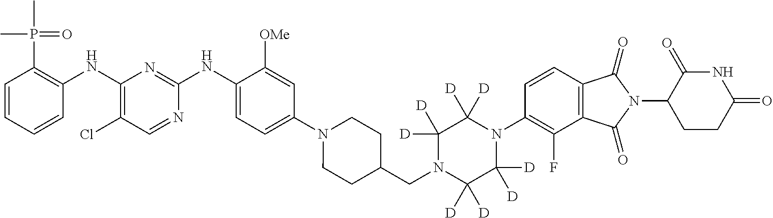 [SMILES: [2H]C1([2H])N(CC2CCN(c3ccc(Nc4ncc(Cl)c(Nc5ccccc5P(C)(C)=O)n4)c(OC)c3)CC2)C([2H])([2H])C([2H])([2H])N(c2ccc3c(c2F)C(=O)N(C2CCC(=O)NC2=O)C3=O)C1([2H])[2H]] | 5-(4-((1-(4-((5-chloro-4-((2- (dimethylphosphoryl)phenyl)amino)pyrimidin- 2-yl)amino)-3-methoxyphenyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 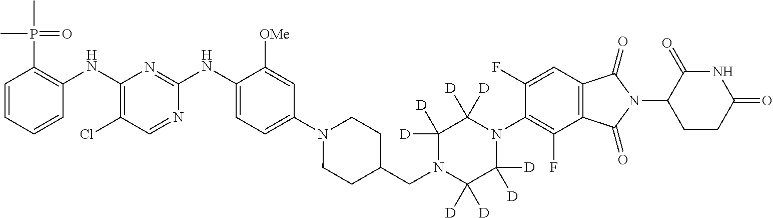 [SMILES: [2H]C1([2H])N(CC2CCN(c3ccc(Nc4ncc(Cl)c(Nc5ccccc5P(C)(C)=O)n4)c(OC)c3)CC2)C([2H])([2H])C([2H])([2H])N(c2c(F)cc3c(c2F)C(=O)N(C2CCC(=O)NC2=O)C3=O)C1([2H])[2H]] | 5-(4-((1-(4-((5-chloro-4-((2- (dimethylphosphoryl)phenyl)amino)pyrimidin- 2-yl)amino)-3-methoxyphenyl)piperidin-4- yl)methyl)piperazin-1-yl-2,2,3,3,5,5,6,6-d₈)-2- (2,6-dioxopiperidin-3-yl)-4,6- difluoroisoindoline-1,3-dione |
| | 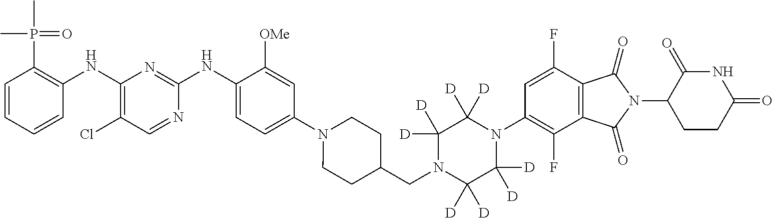 [SMILES: [2H]C1([2H])N(CC2CCN(c3ccc(Nc4ncc(Cl)c(Nc5ccccc5P(C)(C)=O)n4)c(OC)c3)CC2)C([2H])([2H])C([2H])([2H])N(c2cc(F)c3c(c2F)C(=O)N(C2CCC(=O)NC2=O)C3=O)C1([2H])[2H]] | 5-(4-((1-(4-((5-chloro-4-((2- (dimethylphosphoryl)phenyl)amino)pyrimidin- 2-yl)amino)-3-methoxyphenyl)piperidin-4- yl)methyl)piperazin-1-yl-2,2,3,3,5,5,6,6-d₈)-2- (2,6-dioxopiperidin-3-yl)-4,7- difluoroisoindoline-1,3-dione |
| | 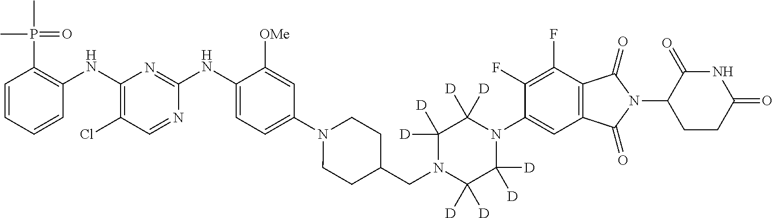 [SMILES: [2H]C1([2H])N(CC2CCN(c3ccc(Nc4ncc(Cl)c(Nc5ccccc5P(C)(C)=O)n4)c(OC)c3)CC2)C([2H])([2H])C([2H])([2H])N(c2cc3c(c(F)c2F)C(=O)N(C2CCC(=O)NC2=O)C3=O)C1([2H])[2H]] | 6-(4-((1-(4-((5-chloro-4-((2- (dimethylphosphoryl)phenyl)amino)pyrimidin- 2-yl)amino)-3-methoxyphenyl)piperidin-4- yl)methyl)piperazin-1-yl-2,2,3,3,5,5,6,6-d₈)-2- (2,6-dioxopiperidin-3-yl)-4,5- difluoroisoindoline-1,3-dione |
| | 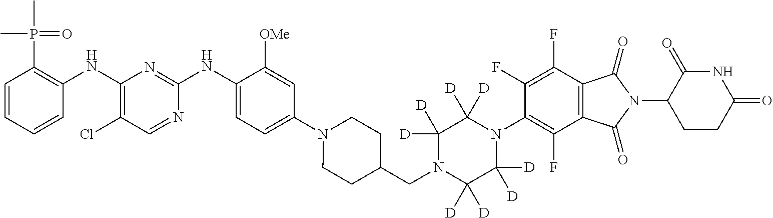 [SMILES: [2H]C1([2H])N(CC2CCN(c3ccc(Nc4ncc(Cl)c(Nc5ccccc5P(C)(C)=O)n4)c(OC)c3)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(4-((5-chloro-4-((2- (dimethylphosphoryl)phenyl)amino)pyrimidin- 2-yl)amino)-3-methoxyphenyl)piperidin-4- yl)methyl)piperazin-1-yl-2,2,3,3,5,5,6,6-d₈)-2- (2,6-dioxopiperidin-3-yl)-4,6,7- trifluoroisoindoline-1,3-dione |
| | 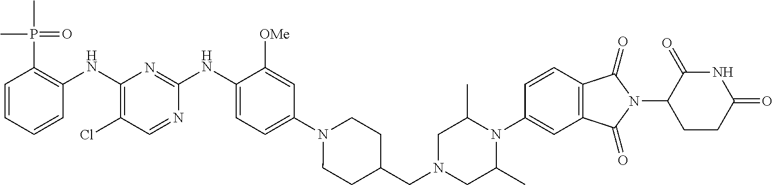 [SMILES: COc1cc(N2CCC(CN3CC(C)N(c4ccc5c(c4)C(=O)N(C4CCC(=O)NC4=O)C5=O)C(C)C3)CC2)ccc1Nc1ncc(Cl)c(Nc2ccccc2P(C)(C)=O)n1] | 5-(4-((1-(4-((5-chloro-4-((2- (dimethylphosphoryl)phenyl)amino)pyrimidin- 2-yl)amino)-3-methoxyphenyl)piperidin-4- yl)methyl)-2,6-dimethylpiperazin-1-yl)-2-(2,6- dioxopiperidin-3-yl)isoindoline-1,3-dione |
| | 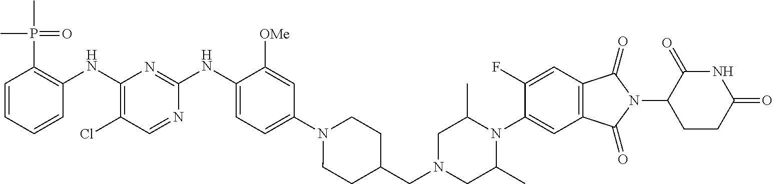 [SMILES: COc1cc(N2CCC(CN3CC(C)N(c4cc5c(cc4F)C(=O)N(C4CCC(=O)NC4=O)C5=O)C(C)C3)CC2)ccc1Nc1ncc(Cl)c(Nc2ccccc2P(C)(C)=O)n1] | 5-(4-((1-(4-((5-chloro-4-((2- (dimethylphosphoryl)phenyl)amino)pyrimidin- 2-yl)amino)-3-methoxyphenyl)piperidin-4- yl)methyl)-2,6-dimethylpiperazin-1-yl)-2-(2,6- dioxopiperidin-3-yl)-6-fluoroisoindoline-1,3- dione |
| | 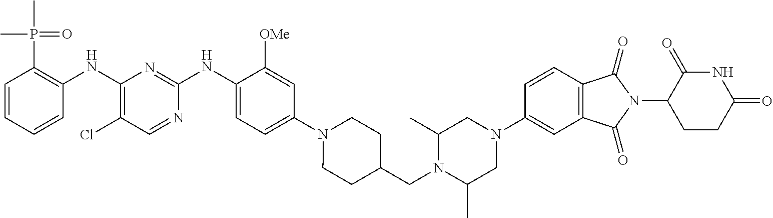 [SMILES: COc1cc(N2CCC(CN3C(C)CN(c4ccc5c(c4)C(=O)N(C4CCC(=O)NC4=O)C5=O)CC3C)CC2)ccc1Nc1ncc(Cl)c(Nc2ccccc2P(C)(C)=O)n1] | 5-(4-((1-(4-((5-chloro-4-((2- (dimethylphosphoryl)phenyl)amino)pyrimidin- 2-yl)amino)-3-methoxyphenyl)piperidin-4- yl)methyl)-3,5-dimethylpiperazin-1-yl)-2-(2,6- dioxopiperidin-3-yl)isoindoline-1,3-dione |
| | 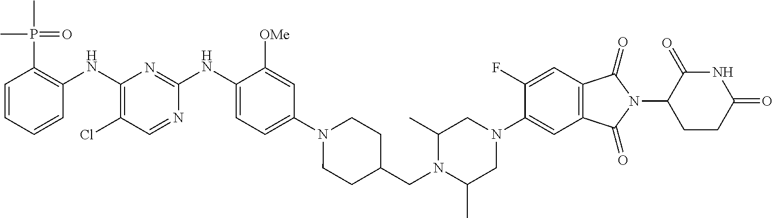 [SMILES: COc1cc(N2CCC(CN3C(C)CN(c4cc5c(cc4F)C(=O)N(C4CCC(=O)NC4=O)C5=O)CC3C)CC2)ccc1Nc1ncc(Cl)c(Nc2ccccc2P(C)(C)=O)n1] | 5-(4-((1-(4-((5-chloro-4-((2- (dimethylphosphoryl)phenyl)amino)pyrimidin- 2-yl)amino)-3-methoxyphenyl)piperidin-4- yl)methyl)-3,5-dimethylpiperazin-1-yl)-2-(2,6- dioxopiperidin-3-yl)-6-fluoroisoindoline-1,3- dione |
| | 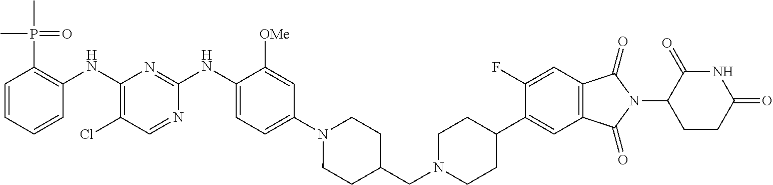 [SMILES: COc1cc(N2CCC(CN3CCC(c4cc5c(cc4F)C(=O)N(C4CCC(=O)NC4=O)C5=O)CC3)CC2)ccc1Nc1ncc(Cl)c(Nc2ccccc2P(C)(C)=O)n1] | 5-(1-((1-(4-((5-chloro-4-((2- (dimethylphosphoryl)phenyl)amino)pyrimidin- 2-yl)amino)-3-methoxyphenyl)piperidin-4- yl)methyl)piperidin-4-yl)-2-(2,6- dioxopiperidin-3-yl)-6-fluoroisoindoline-1,3- dione |
| | 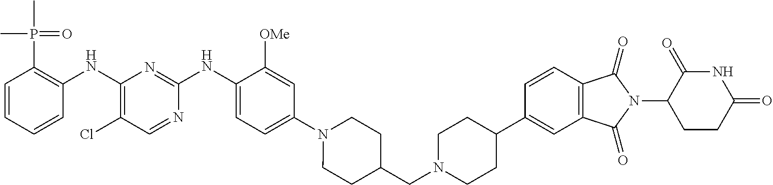 [SMILES: COc1cc(N2CCC(CN3CCC(c4ccc5c(c4)C(=O)N(C4CCC(=O)NC4=O)C5=O)CC3)CC2)ccc1Nc1ncc(Cl)c(Nc2ccccc2P(C)(C)=O)n1] | 5-(1-((1-(4-((5-chloro-4-((2- (dimethylphosphoryl)phenyl)amino)pyrimidin- 2-yl)amino)-3-methoxyphenyl)piperidin-4- yl)methyl)piperidin-4-yl)-2-(2,6- dioxopiperidin-3-yl)isoindoline-1,3-dione |
| | 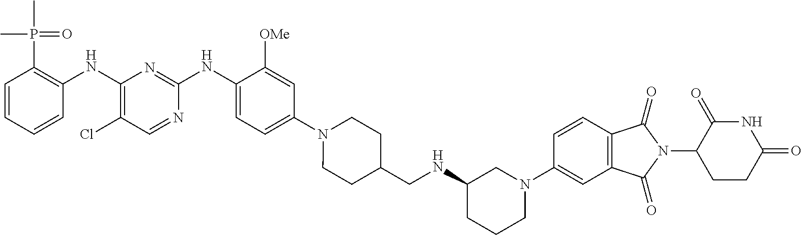 [SMILES: COc1cc(N2CCC(CN[C@@H]3CCCN(c4ccc5c(c4)C(=O)N(C4CCC(=O)NC4=O)C5=O)C3)CC2)ccc1Nc1ncc(Cl)c(Nc2ccccc2P(C)(C)=O)n1] | 5-((R)-3-(((1-(4-((5-chloro-4-((2- (dimethylphosphoryl)phenyl)amino)pyrimidin- 2-yl)amino)-3-methoxyphenyl)piperidin-4- yl)methyl)amino)piperidin-1-yl)-2-(2,6- dioxopiperidin-3-yl)isoindoline-1,3-dione |
| | 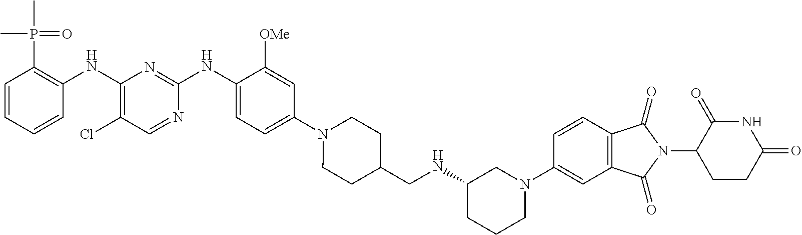 [SMILES: COc1cc(N2CCC(CN[C@H]3CCCN(c4ccc5c(c4)C(=O)N(C4CCC(=O)NC4=O)C5=O)C3)CC2)ccc1Nc1ncc(Cl)c(Nc2ccccc2P(C)(C)=O)n1] | 5-((S)-3-(((1-(4-((5-chloro-4-((2- (dimethylphosphoryl)phenyl)amino)pyrimidin- 2-yl)amino)-3-methoxyphenyl)piperidin-4- yl)methyl)amino)piperidin-1-yl)-2-(2,6- dioxopiperidin-3-yl)isoindoline-1,3-dione |
| | 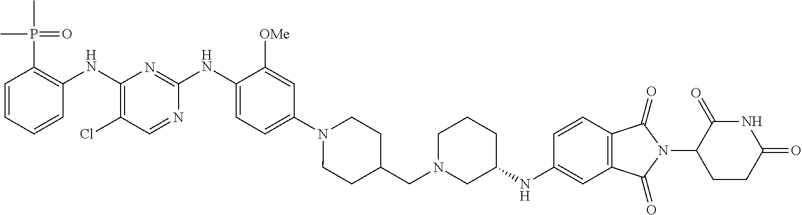 [SMILES: COc1cc(N2CCC(CN3CCC[C@H](Nc4ccc5c(c4)C(=O)N(C4CCC(=O)NC4=O)C5=O)C3)CC2)ccc1Nc1ncc(Cl)c(Nc2ccccc2P(C)(C)=O)n1] | 5-(((S)-1-((1-(4-((5-chloro-4-((2- (dimethylphosphoryl)phenyl)amino)pyrimidin- 2-yl)amino)-3-methoxyphenyl)piperidin-4- yl)methyl)piperidin-3-yl)amino)-2-(2,6- dioxopiperidin-3-yl)isoindoline-1,3-dione |
| | 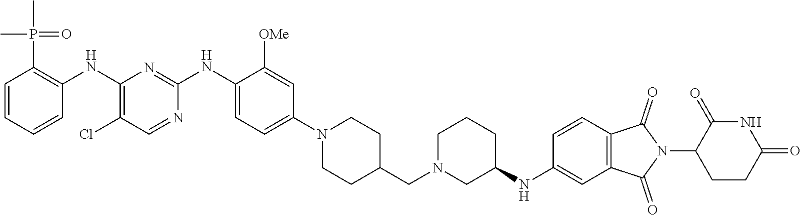 [SMILES: COc1cc(N2CCC(CN3CCC[C@@H](Nc4ccc5c(c4)C(=O)N(C4CCC(=O)NC4=O)C5=O)C3)CC2)ccc1Nc1ncc(Cl)c(Nc2ccccc2P(C)(C)=O)n1] | 5-(((R)-1-((1-(4-((5-chloro-4-((2- (dimethylphosphoryl)phenyl)amino)pyrimidin- 2-yl)amino)-3-methoxyphenyl)piperidin-4- yl)methyl)piperidin-3-yl)amino)-2-(2,6- dioxopiperidin-3-yl)isoindoline-1,3-dione |
| | 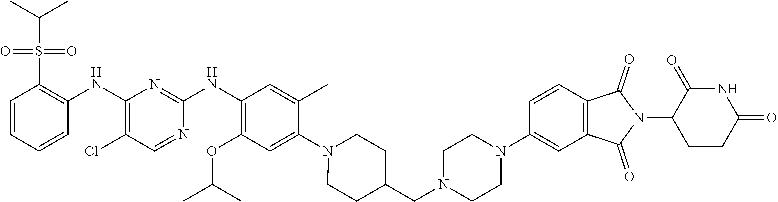 [SMILES: Cc1cc(Nc2ncc(Cl)c(Nc3ccccc3S(=O)(=O)C(C)C)n2)c(OC(C)C)cc1N1CCC(CN2CCN(c3ccc4c(c3)C(=O)N(C3CCC(=O)NC3=O)C4=O)CC2)CC1] | 5-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)piperazin- 1-yl)-2-(2,6-dioxopiperidin-3-yl)isoindoline- 1,3-dione |
| | 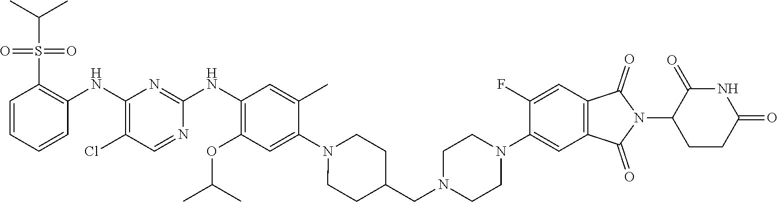 [SMILES: Cc1cc(Nc2ncc(Cl)c(Nc3ccccc3S(=O)(=O)C(C)C)n2)c(OC(C)C)cc1N1CCC(CN2CCN(c3cc4c(cc3F)C(=O)N(C3CCC(=O)NC3=O)C4=O)CC2)CC1] | 5-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)piperazin- 1-yl)-2-(2,6-dioxopiperidin-3-yl)-6- fluoroisoindoline-1,3-dione |
| | 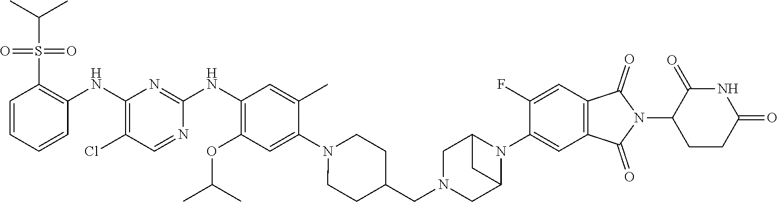 [SMILES: Cc1cc(Nc2ncc(Cl)c(Nc3ccccc3S(=O)(=O)C(C)C)n2)c(OC(C)C)cc1N1CCC(CN2CC3CC(C2)N3c2cc3c(cc2F)C(=O)N(C2CCC(=O)NC2=O)C3=O)CC1] | 5-(3-((1-(4-((5-chloro-4-((2- (isopropylsulfonyl)phenyl)amino)pyrimidin-2- yl)amino)-5-isopropoxy-2- methylphenyl)piperidin-4-yl)methyl)-3,6- diazabicyclo[3.1.1]heptan-6-yl)-2-(2,6- dioxopiperidin-3-yl)-6-fluoroisoindoline-1,3- dione |
| | 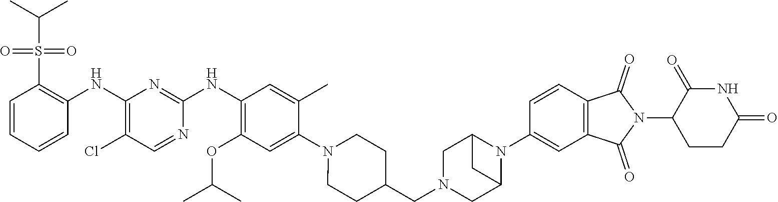 [SMILES: Cc1cc(Nc2ncc(Cl)c(Nc3ccccc3S(=O)(=O)C(C)C)n2)c(OC(C)C)cc1N1CCC(CN2CC3CC(C2)N3c2ccc3c(c2)C(=O)N(C2CCC(=O)NC2=O)C3=O)CC1] | 5-(3-((1-(4-((5-chloro-4-((2- (isopropylsulfonyl)phenyl)amino)pyrimidin-2- yl)amino)-5-isopropoxy-2- methylphenyl)piperidin-4-yl)methyl)-3,6- diazabicyclo[3.1.1]heptan-6-yl)-2-(2,6- dioxopiperidin-3-yl)isoindoline-1,3-dione |
| | 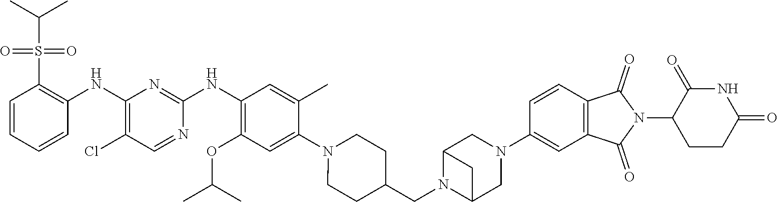 [SMILES: Cc1cc(Nc2ncc(Cl)c(Nc3ccccc3S(=O)(=O)C(C)C)n2)c(OC(C)C)cc1N1CCC(CN2C3CC2CN(c2ccc4c(c2)C(=O)N(C2CCC(=O)NC2=O)C4=O)C3)CC1] | 5-(6-((1-(4-((5-chloro-4-((2- (isopropylsulfonyl)phenyl)amino)pyrimidin-2- yl)amino)-5-isopropoxy-2- methylphenyl)piperidin-4-yl)methyl)-3,6- diazabicyclo[3.1.1]heptan-3-yl)-2-(2,6- dioxopiperidin-3-yl)isoindoline-1,3-dione |
| | 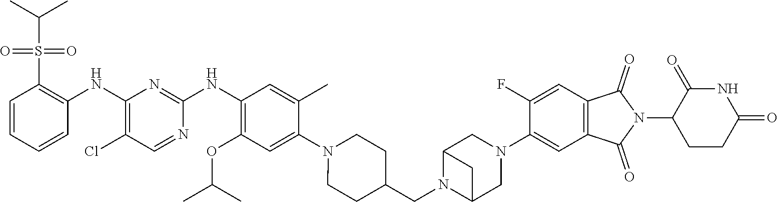 [SMILES: Cc1cc(Nc2ncc(Cl)c(Nc3ccccc3S(=O)(=O)C(C)C)n2)c(OC(C)C)cc1N1CCC(CN2C3CC2CN(c2cc4c(cc2F)C(=O)N(C2CCC(=O)NC2=O)C4=O)C3)CC1] | 5-(6-((1-(4-((5-chloro-4-((2- (isopropylsulfonyl)phenyl)amino)pyrimidin-2- yl)amino)-5-isopropoxy-2- methylphenyl)piperidin-4-yl)methyl)-3,6- diazabicyclo[3.1.1]heptan-3-yl)-2-(2,6- dioxopiperidin-3-yl)-6-fluoroisoindoline-1,3- dione |
| | 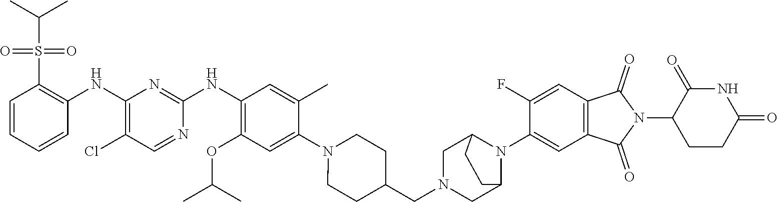 [SMILES: Cc1cc(Nc2ncc(Cl)c(Nc3ccccc3S(=O)(=O)C(C)C)n2)c(OC(C)C)cc1N1CCC(CN2CC3CCC(C2)N3c2cc3c(cc2F)C(=O)N(C2CCC(=O)NC2=O)C3=O)CC1] | 5-(3-((1-(4-((5-chloro-4-((2- (isopropylsulfonyl)phenyl)amino)pyrimidin-2- yl)amino)-5-isopropoxy-2- methylphenyl)piperidin-4-yl)methyl)-3,8- diazabicyclo[3.2.1]octan-8-yl)-2-(2,6- dioxopiperidin-3-yl)-6-fluoroisoindoline-1,3- dione |
| | 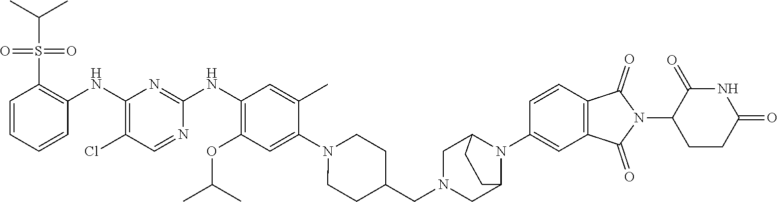 [SMILES: Cc1cc(Nc2ncc(Cl)c(Nc3ccccc3S(=O)(=O)C(C)C)n2)c(OC(C)C)cc1N1CCC(CN2CC3CCC(C2)N3c2ccc3c(c2)C(=O)N(C2CCC(=O)NC2=O)C3=O)CC1] | 5-(3-((1-(4-((5-chloro-4-((2- (isopropylsulfonyl)phenyl)amino)pyrimidin-2- yl)amino)-5-isopropoxy-2- methylphenyl)piperidin-4-yl)methyl)-3,8- diazabicyclo[3.2.1]octan-8-yl)-2-(2,6- dioxopiperidin-3-yl)isoindoline-1,3-dione |
| | 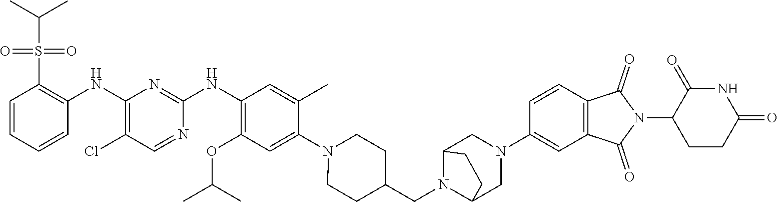 [SMILES: Cc1cc(Nc2ncc(Cl)c(Nc3ccccc3S(=O)(=O)C(C)C)n2)c(OC(C)C)cc1N1CCC(CN2C3CCC2CN(c2ccc4c(c2)C(=O)N(C2CCC(=O)NC2=O)C4=O)C3)CC1] | 5-(8-((1-(4-((5-chloro-4-((2- (isopropylsulfonyl)phenyl)amino)pyrimidin-2- yl)amino)-5-isopropoxy-2- methylphenyl)piperidin-4-yl)methyl)-3,8- diazabicyclo[3.2.1]octan-3-yl)-2-(2,6- dioxopiperidin-3-yl)isoindoline-1,3-dione |
| | 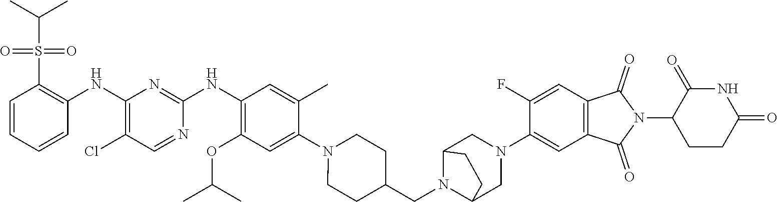 [SMILES: Cc1cc(Nc2ncc(Cl)c(Nc3ccccc3S(=O)(=O)C(C)C)n2)c(OC(C)C)cc1N1CCC(CN2C3CCC2CN(c2cc4c(cc2F)C(=O)N(C2CCC(=O)NC2=O)C4=O)C3)CC1] | 5-(8-((1-(4-((5-chloro-4-((2- (isopropylsulfonyl)phenyl)amino)pyrimidin-2- yl)amino)-5-isopropoxy-2- methylphenyl)piperidin-4-yl)methyl)-3,8- diazabicyclo[3.2.1]octan-3-yl)-2-(2,6- dioxopiperidin-3-yl)-6-fluoroisoindoline-1,3- dione |
| | 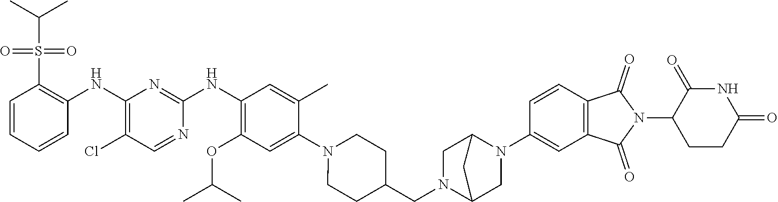 [SMILES: Cc1cc(Nc2ncc(Cl)c(Nc3ccccc3S(=O)(=O)C(C)C)n2)c(OC(C)C)cc1N1CCC(CN2CC3CC2CN3c2ccc3c(c2)C(=O)N(C2CCC(=O)NC2=O)C3=O)CC1] | 5-(5-((1-(4-((5-chloro-4-((2- (isopropylsulfonyl)phenyl)amino)pyrimidin-2- yl)amino)-5-isopropoxy-2- methylphenyl)piperidin-4-yl)methyl)-2,5- diazabicyclo[2.2.1]heptan-2-yl)-2-(2,6- dioxopiperidin-3-yl)isoindoline-1,3-dione |
| | 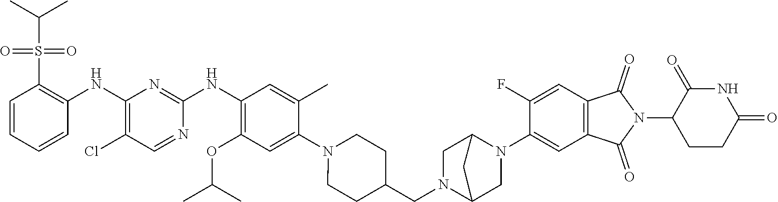 [SMILES: Cc1cc(Nc2ncc(Cl)c(Nc3ccccc3S(=O)(=O)C(C)C)n2)c(OC(C)C)cc1N1CCC(CN2CC3CC2CN3c2cc3c(cc2F)C(=O)N(C2CCC(=O)NC2=O)C3=O)CC1] | 5-(5-((1-(4-((5-chloro-4-((2- (isopropylsulfonyl)phenyl)amino)pyrimidin-2- yl)amino)-5-isopropoxy-2- methylphenyl)piperidin-4-yl)methyl)-2,5- diazabicyclo[2.2.1]heptan-2-yl)-2-(2,6- dioxopiperidin-3-yl)-6-fluoroisoindoline-1,3- dione |
| | 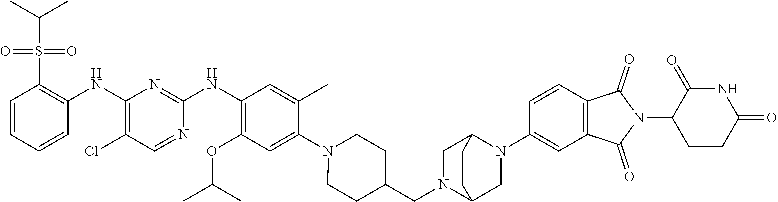 [SMILES: Cc1cc(Nc2ncc(Cl)c(Nc3ccccc3S(=O)(=O)C(C)C)n2)c(OC(C)C)cc1N1CCC(CN2CC3CCC2CN3c2ccc3c(c2)C(=O)N(C2CCC(=O)NC2=O)C3=O)CC1] | 5-(5-((1-(4-((5-chloro-4-((2- (isopropylsulfonyl)phenyl)amino)pyrimidin-2- yl)amino)-5-isopropoxy-2- methylphenyl)piperidin-4-yl)methyl)-2,5- diazabicyclo[2.2.2]octan-2-yl)-2-(2,6- dioxopiperidin-3-yl)isoindoline-1,3-dione |
| | 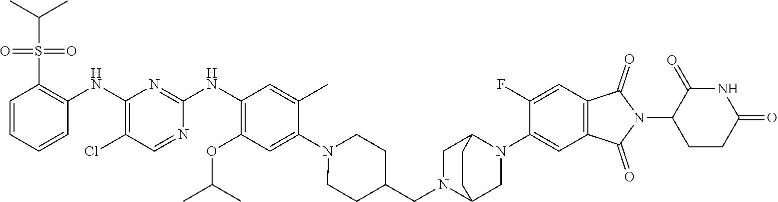 [SMILES: Cc1cc(Nc2ncc(Cl)c(Nc3ccccc3S(=O)(=O)C(C)C)n2)c(OC(C)C)cc1N1CCC(CN2CC3CCC2CN3c2cc3c(cc2F)C(=O)N(C2CCC(=O)NC2=O)C3=O)CC1] | 5-(5-((1-(4-((5-chloro-4-((2- (isopropylsulfonyl)phenyl)amino)pyrimidin-2- yl)amino)-5-isopropoxy-2- methylphenyl)piperidin-4-yl)methyl)-2,5- diazabicyclo[2.2.2]octan-2-yl)-2-(2,6- dioxopiperidin-3-yl)-6-fluoroisoindoline-1,3- dione |
| | 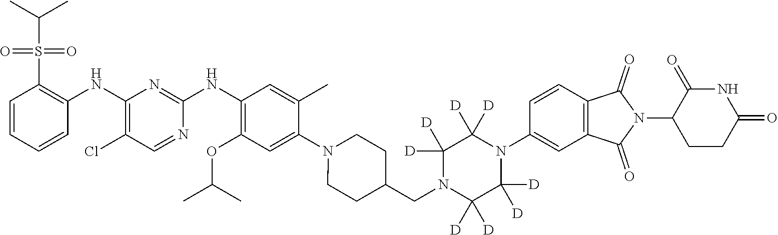 [SMILES: [2H]C1([2H])N(CC2CCN(c3cc(OC(C)C)c(Nc4ncc(Cl)c(Nc5ccccc5S(=O)(=O)C(C)C)n4)cc3C)CC2)C([2H])([2H])C([2H])([2H])N(c2ccc3c(c2)C(=O)N(C2CCC(=O)NC2=O)C3=O)C1([2H])[2H]] | 5-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)piperazin- 1-yl-2,2,3,3,5,5,6,6-d₈)-2-(2,6-dioxopiperidin- 3-yl)isoindoline-1,3-dione |
| | 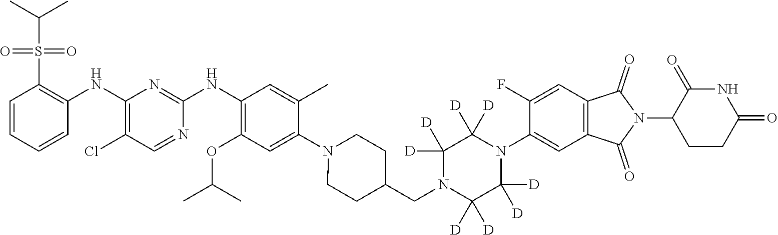 [SMILES: [2H]C1([2H])N(CC2CCN(c3cc(OC(C)C)c(Nc4ncc(Cl)c(Nc5ccccc5S(=O)(=O)C(C)C)n4)cc3C)CC2)C([2H])([2H])C([2H])([2H])N(c2cc3c(cc2F)C(=O)N(C2CCC(=O)NC2=O)C3=O)C1([2H])[2H]] | 5-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)piperazin- 1-yl-2,2,3,3,5,5,6,6-d₈)-2-(2,6-dioxopiperidin- 3-yl)-6-fluoroisoindoline-1,3-dione |
| | 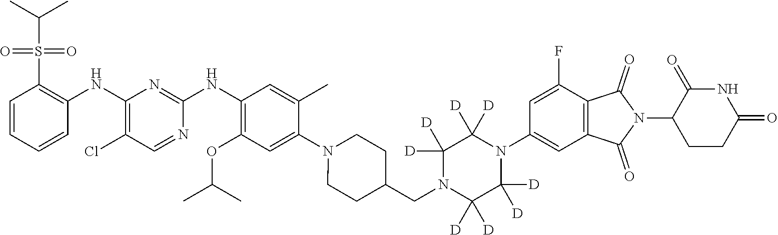 [SMILES: [2H]C1([2H])N(CC2CCN(c3cc(OC(C)C)c(Nc4ncc(Cl)c(Nc5ccccc5S(=O)(=O)C(C)C)n4)cc3C)CC2)C([2H])([2H])C([2H])([2H])N(c2cc(F)c3c(c2)C(=O)N(C2CCC(=O)NC2=O)C3=O)C1([2H])[2H]] | 6-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)piperazin- 1-yl-2,2,3,3,5,5,6,6-d₈)-2-(2,6-dioxopiperidin- 3-yl)-4-fluoroisoindoline-1,3-dione |
| | 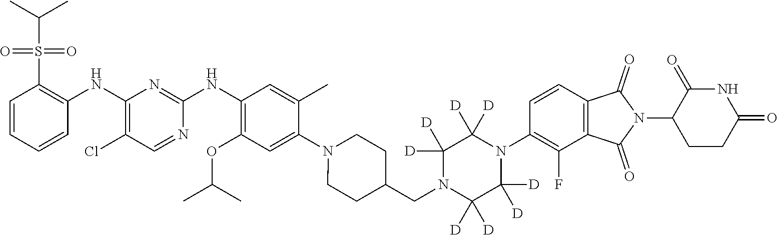 [SMILES: [2H]C1([2H])N(CC2CCN(c3cc(OC(C)C)c(Nc4ncc(Cl)c(Nc5ccccc5S(=O)(=O)C(C)C)n4)cc3C)CC2)C([2H])([2H])C([2H])([2H])N(c2ccc3c(c2F)C(=O)N(C2CCC(=O)NC2=O)C3=O)C1([2H])[2H]] | 5-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)piperazin- 1-yl-2,2,3,3,5,5,6,6-d₈)-2-(2,6-dioxopiperidin- 3-yl)-4-fluoroisoindoline-1,3-dione |
| | 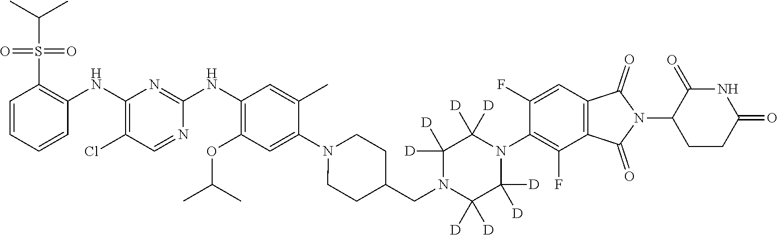 [SMILES: [2H]C1([2H])N(CC2CCN(c3cc(OC(C)C)c(Nc4ncc(Cl)c(Nc5ccccc5S(=O)(=O)C(C)C)n4)cc3C)CC2)C([2H])([2H])C([2H])([2H])N(c2c(F)cc3c(c2F)C(=O)N(C2CCC(=O)NC2=O)C3=O)C1([2H])[2H]] | 5-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)piperazin- 1-yl-2,2,3,3,5,5,6,6-d₈)-2-(2,6-dioxopiperidin- 3-yl)-4,6-difluoroisoindoline-1,3-dione |
| | 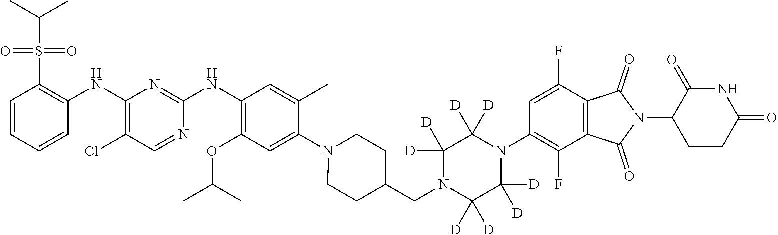 [SMILES: [2H]C1([2H])N(CC2CCN(c3cc(OC(C)C)c(Nc4ncc(Cl)c(Nc5ccccc5S(=O)(=O)C(C)C)n4)cc3C)CC2)C([2H])([2H])C([2H])([2H])N(c2cc(F)c3c(c2F)C(=O)N(C2CCC(=O)NC2=O)C3=O)C1([2H])[2H]] | 5-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)piperazin- 1-yl-2,2,3,3,5,5,6,6-d₈)-2-(2,6-dioxopiperidin- 3-yl)-4,7-difluoroisoindoline-1,3-dione |
| | 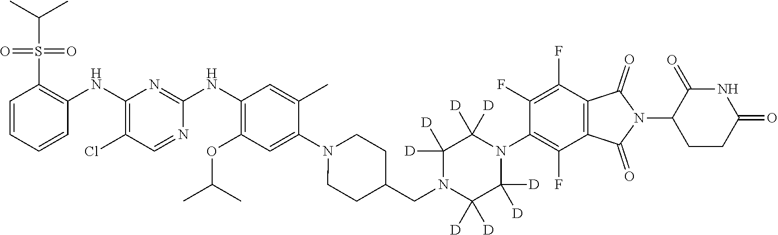 [SMILES: [2H]C1([2H])N(CC2CCN(c3cc(OC(C)C)c(Nc4ncc(Cl)c(Nc5ccccc5S(=O)(=O)C(C)C)n4)cc3C)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)piperazin- 1-yl-2,2,3,3,5,5,6,6-d₈)-2-(2,6-dioxopiperidin- 3-yl)-4,6,7-trifluoroisoindoline-1,3-dione |
| | 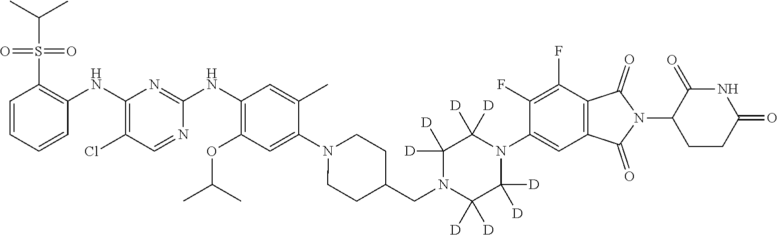 [SMILES: [2H]C1([2H])N(CC2CCN(c3cc(OC(C)C)c(Nc4ncc(Cl)c(Nc5ccccc5S(=O)(=O)C(C)C)n4)cc3C)CC2)C([2H])([2H])C([2H])([2H])N(c2cc3c(c(F)c2F)C(=O)N(C2CCC(=O)NC2=O)C3=O)C1([2H])[2H]] | 6-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)piperazin- 1-yl-2,2,3,3,5,5,6,6-d₈)-2-(2,6-dioxopiperidin- 3-yl)-4,5-difluoroisoindoline-1,3-dione |
| | 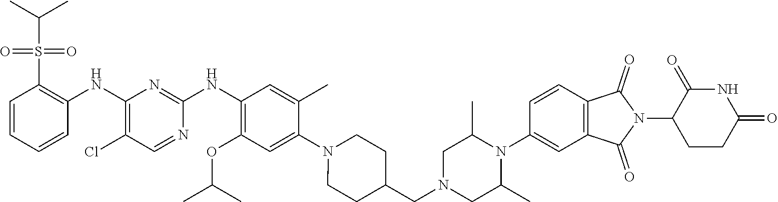 [SMILES: Cc1cc(Nc2ncc(Cl)c(Nc3ccccc3S(=O)(=O)C(C)C)n2)c(OC(C)C)cc1N1CCC(CN2CC(C)N(c3ccc4c(c3)C(=O)N(C3CCC(=O)NC3=O)C4=O)C(C)C2)CC1] | 5-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)-2,6- dimethylpiperazin-1-yl)-2-(2,6-dioxopiperidin- 3-yl)isoindoline-1,3-dione |
| | 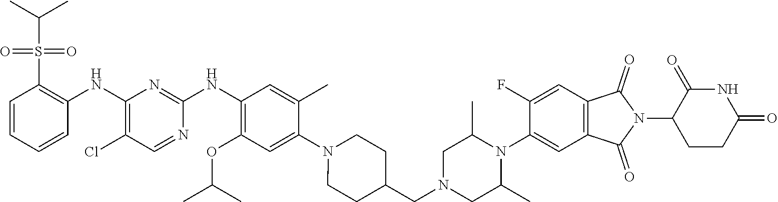 [SMILES: Cc1cc(Nc2ncc(Cl)c(Nc3ccccc3S(=O)(=O)C(C)C)n2)c(OC(C)C)cc1N1CCC(CN2CC(C)N(c3cc4c(cc3F)C(=O)N(C3CCC(=O)NC3=O)C4=O)C(C)C2)CC1] | 5-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)-2,6- dimethylpiperazin-1-yl)-2-(2,6-dioxopiperidin- 3-yl)-6-fluoroisoindoline-1,3-dione |
| | 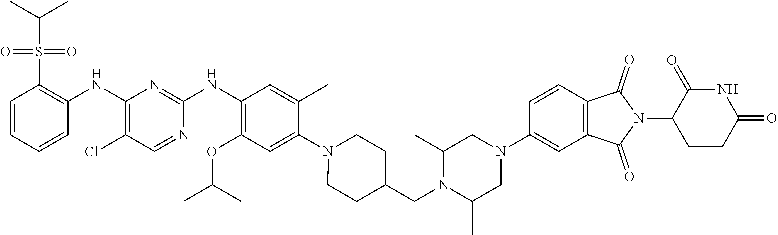 [SMILES: Cc1cc(Nc2ncc(Cl)c(Nc3ccccc3S(=O)(=O)C(C)C)n2)c(OC(C)C)cc1N1CCC(CN2C(C)CN(c3ccc4c(c3)C(=O)N(C3CCC(=O)NC3=O)C4=O)CC2C)CC1] | 5-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)-3,5- dimethylpiperazin-1-yl)-2-(2,6-dioxopiperidin- 3-yl)isoindoline-1,3-dione |
| | 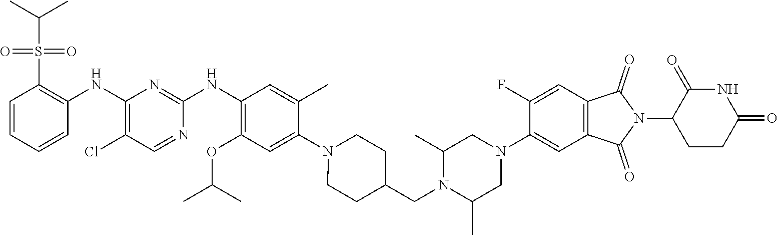 [SMILES: Cc1cc(Nc2ncc(Cl)c(Nc3ccccc3S(=O)(=O)C(C)C)n2)c(OC(C)C)cc1N1CCC(CN2C(C)CN(c3cc4c(cc3F)C(=O)N(C3CCC(=O)NC3=O)C4=O)CC2C)CC1] | 5-(4-((1-(4-((5-chloro-4-((2- (isopropylsulfonyl)phenyl)amino)pyrimidin-2- yl)amino)-5-isopropoxy-2- methylphenyl)piperidin-4-yl)methyl)-3,5- dimethylpiperazin-1-yl)-2-(2,6-dioxopiperidin- 3-yl)-6-fluoroisoindoline-1,3-dione |
| | 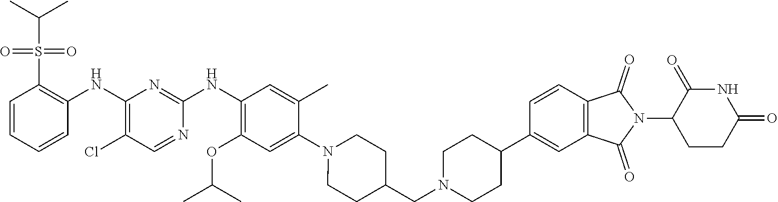 [SMILES: Cc1cc(Nc2ncc(Cl)c(Nc3ccccc3S(=O)(=O)C(C)C)n2)c(OC(C)C)cc1N1CCC(CN2CCC(c3ccc4c(c3)C(=O)N(C3CCC(=O)NC3=O)C4=O)CC2)CC1] | 5-(1-((1-(4-((5-chloro-4-((2- (isopropylsulfonyl)phenyl)amino)pyrimidin-2- yl)amino)-5-isopropoxy-2- methylphenyl)piperidin-4-yl)methyl)piperidin- 4-yl)-2-(2,6-dioxopiperidin-3-yl)isoindoline- 1,3-dione |
| | 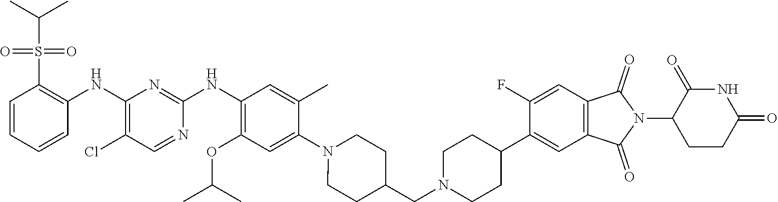 [SMILES: Cc1cc(Nc2ncc(Cl)c(Nc3ccccc3S(=O)(=O)C(C)C)n2)c(OC(C)C)cc1N1CCC(CN2CCC(c3cc4c(cc3F)C(=O)N(C3CCC(=O)NC3=O)C4=O)CC2)CC1] | 5-(1-((1-(4-((5-chloro-4-((2- (isopropylsulfonyl)phenyl)amino)pyrimidin-2- yl)amino)-5-isopropoxy-2- methylphenyl)piperidin-4-yl)methyl)piperidin- 4-yl)-2-(2,6-dioxopiperidin-3-yl)-6- fluoroisoindoline-1,3-dione |
| | 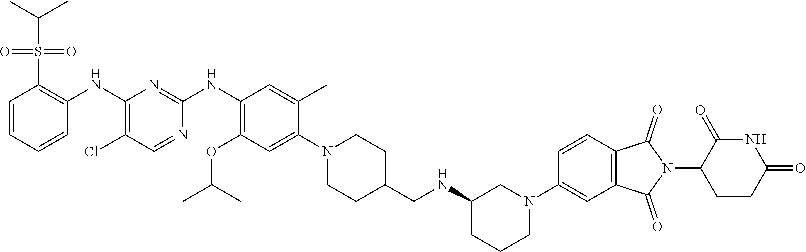 [SMILES: Cc1cc(Nc2ncc(Cl)c(Nc3ccccc3S(=O)(=O)C(C)C)n2)c(OC(C)C)cc1N1CCC(CN[C@@H]2CCCN(c3ccc4c(c3)C(=O)N(C3CCC(=O)NC3=O)C4=O)C2)CC1] | 5-((R)-3-(((1-(4-((5-chloro-4-((2- (isopropylsulfonyl)phenyl)amino)pyrimidin-2- yl)amino)-5-isopropoxy-2- methylphenyl)piperidin-4- yl)methyl)amino)piperidin-1-yl)-2-(2,6- dioxopiperidin-3-yl)isoindoline-1,3-dione |
| | 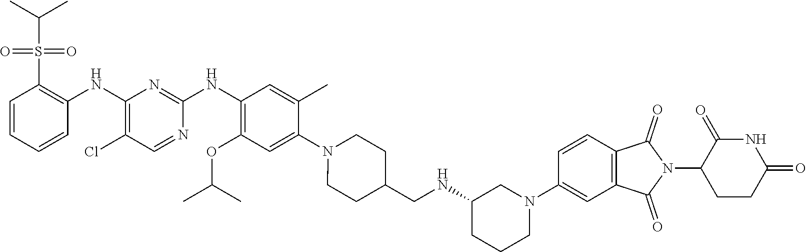 [SMILES: Cc1cc(Nc2ncc(Cl)c(Nc3ccccc3S(=O)(=O)C(C)C)n2)c(OC(C)C)cc1N1CCC(CN[C@H]2CCCN(c3ccc4c(c3)C(=O)N(C3CCC(=O)NC3=O)C4=O)C2)CC1] | 5-((S)-3-(((1-(4-((5-chloro-4-((2- (isopropylsulfonyl)phenyl)amino)pyrimidin-2- yl)amino)-5-isopropoxy-2- methylphenyl)piperidin-4- yl)methyl)amino)piperidin-1-yl)-2-(2,6- dioxopiperidin-3-yl)isoindoline-1,3-dione |
| | 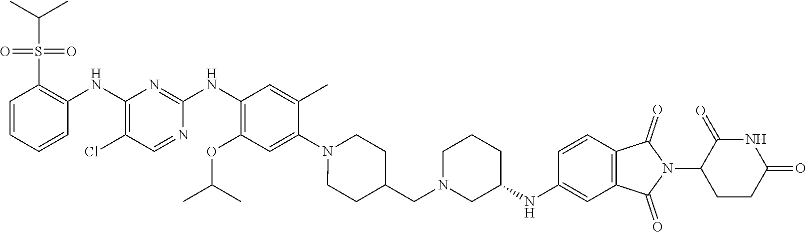 [SMILES: Cc1cc(Nc2ncc(Cl)c(Nc3ccccc3S(=O)(=O)C(C)C)n2)c(OC(C)C)cc1N1CCC(CN2CCC[C@H](Nc3ccc4c(c3)C(=O)N(C3CCC(=O)NC3=O)C4=O)C2)CC1] | 5-(((S)-1-((1-(4-((5-chloro-4-((2- (isopropylsulfonyl)phenyl)amino)pyrimidin-2- yl)amino)-5-isopropoxy-2- methylphenyl)piperidin-4-yl)methyl)piperidin- 3-yl)amino)-2-(2,6-dioxopiperidin-3- yl)isoindoline-1,3-dione |
| | 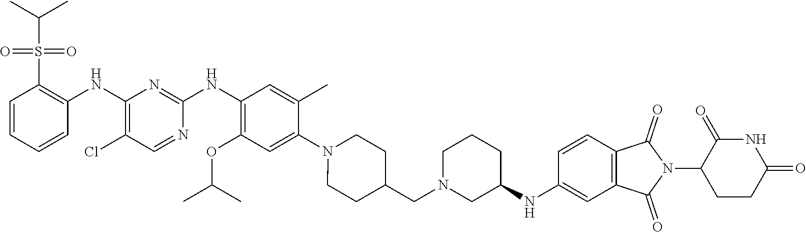 [SMILES: Cc1cc(Nc2ncc(Cl)c(Nc3ccccc3S(=O)(=O)C(C)C)n2)c(OC(C)C)cc1N1CCC(CN2CCC[C@@H](Nc3ccc4c(c3)C(=O)N(C3CCC(=O)NC3=O)C4=O)C2)CC1] | 5-(((R)-1-((1-(4-((5-chloro-4-((2- (isopropylsulfonyl)phenyl)amino)pyrimidin-2- yl)amino)-5-isopropoxy-2- methylphenyl)piperidin-4-yl)methyl)piperidin- 3-yl)amino)-2-(2,6-dioxopiperidin-3- yl)isoindoline-1,3-dione |
| | 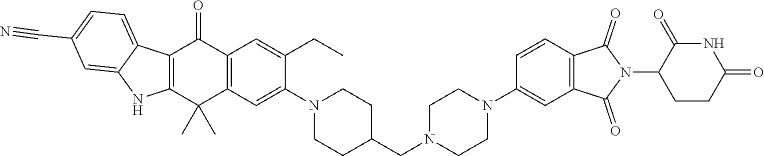 [SMILES: CCc1cc2c(cc1N1CCC(CN3CCN(c4ccc5c(c4)C(=O)N(C4CCC(=O)NC4=O)C5=O)CC3)CC1)C(C)(C)c1[nH]c3cc(C#N)ccc3c1C2=O] | 8-(4-((4-(2-(2,6-dioxopiperidin-3-yl)-1,3- dioxoisoindolin-5-yl)piperazin-1- yl)methyl)piperidin-1-yl)-9-ethyl-6,6-dimethyl- 11-oxo-6,11-dihydro-5H-benzo[b]carbazole-3- carbonitrile |
| | 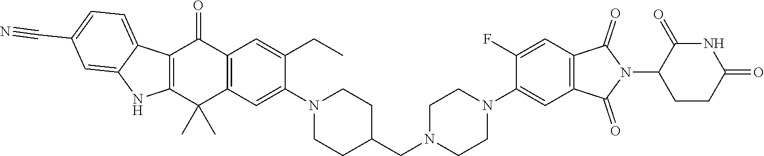 [SMILES: CCc1cc2c(cc1N1CCC(CN3CCN(c4cc5c(cc4F)C(=O)N(C4CCC(=O)NC4=O)C5=O)CC3)CC1)C(C)(C)c1[nH]c3cc(C#N)ccc3c1C2=O] | 8-(4-((4-(2-(2,6-dioxopiperidin-3-yl)-6-fluoro- 1,3-dioxoisoindolin-5-yl)piperazin-1- yl)methyl)piperidin-1-yl)-9-ethyl-6,6-dimethyl- 11-oxo-6,11-dihydro-5H-benzo[b]carbazole-3- carbonitrile |
| | 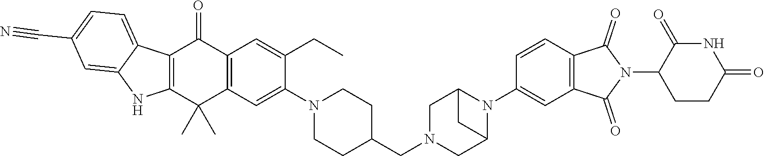 [SMILES: CCc1cc2c(cc1N1CCC(CN3CC4CC(C3)N4c3ccc4c(c3)C(=O)N(C3CCC(=O)NC3=O)C4=O)CC1)C(C)(C)c1[nH]c3cc(C#N)ccc3c1C2=O] | 8-(4-((6-(2-(2,6-dioxopiperidin-3-yl)-1,3- dioxoisoindolin-5-yl)-3,6- diazabicyclo[3.1.1]heptan-3- yl)methyl)piperidin-1-yl)-9-ethyl-6,6-dimethyl- 11-oxo-6,11-dihydro-5H-benzo[b]carbazole-3- carbonitrile |
| | 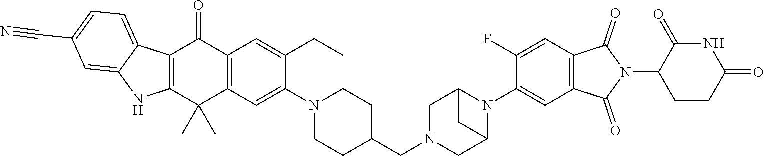 [SMILES: CCc1cc2c(cc1N1CCC(CN3CC4CC(C3)N4c3cc4c(cc3F)C(=O)N(C3CCC(=O)NC3=O)C4=O)CC1)C(C)(C)c1[nH]c3cc(C#N)ccc3c1C2=O] | 8-(4-((6-(2-(2,6-dioxopiperidin-3-yl)-6-fluoro- 1,3-dioxoisoindolin-5-yl)-3,6- diazabicyclo[3.1.1]heptan-3- yl)methyl)piperidin-1-yl)-9-ethyl-6,6-dimethyl- 11-oxo-6,11-dihydro-5H-benzo[b]carbazole-3- carbonitrile |
| | 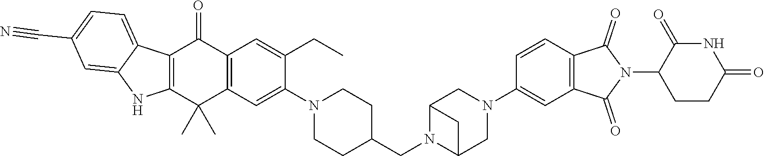 [SMILES: CCc1cc2c(cc1N1CCC(CN3C4CC3CN(c3ccc5c(c3)C(=O)N(C3CCC(=O)NC3=O)C5=O)C4)CC1)C(C)(C)c1[nH]c3cc(C#N)ccc3c1C2=O] | 8-(4-((3-(2-(2,6-dioxopiperidin-3-yl)-1,3- dioxoisoindolin-5-yl)-3,6- diazabicyclo[3.1.1]heptan-6- yl)methyl)piperidin-1-yl)-9-ethyl-6,6-dimethyl- 11-oxo-6,11-dihydro-5H-benzo[b]carbazole-3- carbonitrile |
| | 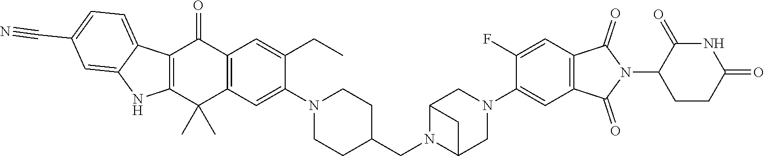 [SMILES: CCc1cc2c(cc1N1CCC(CN3C4CC3CN(c3cc5c(cc3F)C(=O)N(C3CCC(=O)NC3=O)C5=O)C4)CC1)C(C)(C)c1[nH]c3cc(C#N)ccc3c1C2=O] | 8-(4-((3-(2-(2,6-dioxopiperidin-3-yl)-6-fluoro- 1,3-dioxoisoindolin-5-yl)-3,6- diazabicyclo[3.1.1]heptan-6- yl)methyl)piperidin-1-yl)-9-ethyl-6,6-dimethyl- 11-oxo-6,11-dihydro-5H-benzo[b]carbazole-3- carbonitrile |
| | 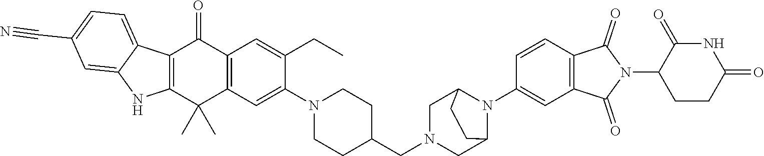 [SMILES: CCc1cc2c(cc1N1CCC(CN3CC4CCC(C3)N4c3ccc4c(c3)C(=O)N(C3CCC(=O)NC3=O)C4=O)CC1)C(C)(C)c1[nH]c3cc(C#N)ccc3c1C2=O] | 8-(4-((8-(2-(2,6-dioxopiperidin-3-yl)-1,3- dioxoisoindolin-5-yl)-3,8- diazabicyclo[3.2.1]octan-3-yl)methyl)piperidin- 1-yl)-9-ethyl-6,6-dimethyl-11-oxo-6,11- dihydro-5H-benzo[b]carbazole-3-carbonitrile |
| | 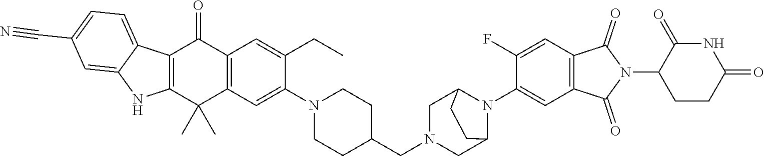 [SMILES: CCc1cc2c(cc1N1CCC(CN3CC4CCC(C3)N4c3cc4c(cc3F)C(=O)N(C3CCC(=O)NC3=O)C4=O)CC1)C(C)(C)c1[nH]c3cc(C#N)ccc3c1C2=O] | 8-(4-((8-(2-(2,6-dioxopiperidin-3-yl)-6-fluoro- 1,3-dioxoisoindolin-5-yl)-3,8- diazabicyclo[3.2.1]octan-3-yl)methyl)piperidin- 1-yl)-9-ethyl-6,6-dimethyl-11-oxo-6,11- dihydro-5H-benzo[b]carbazole-3-carbonitrile |
| | 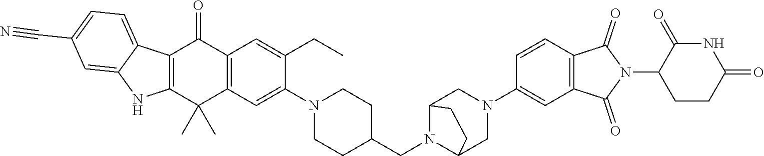 [SMILES: CCc1cc2c(cc1N1CCC(CN3C4CCC3CN(c3ccc5c(c3)C(=O)N(C3CCC(=O)NC3=O)C5=O)C4)CC1)C(C)(C)c1[nH]c3cc(C#N)ccc3c1C2=O] | 8-(4-((3-(2-(2,6-dioxopiperidin-3-yl)-1,3- dioxoisoindolin-5-yl)-3,8- diazabicyclo[3.2.1]octan-8-yl)methyl)piperidin- 1-yl)-9-ethyl-6,6-dimethyl-11-oxo-6,11- dihydro-5H-benzo[b]carbazole-3-carbonitrile |
| | 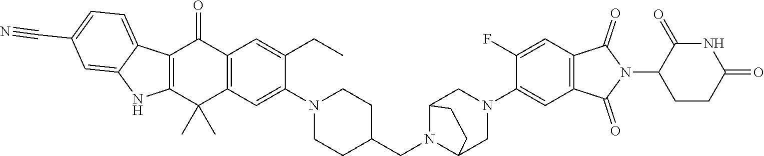 [SMILES: CCc1cc2c(cc1N1CCC(CN3C4CCC3CN(c3cc5c(cc3F)C(=O)N(C3CCC(=O)NC3=O)C5=O)C4)CC1)C(C)(C)c1[nH]c3cc(C#N)ccc3c1C2=O] | 8-(4-((3-(2-(2,6-dioxopiperidin-3-yl)-6-fluoro- 1,3-dioxoisoindolin-5-yl)-3,8- diazabicyclo[3.2.1]octan-8-yl)methyl)piperidin- 1-yl)-9-ethyl-6,6-dimethyl-11-oxo-6,11- dihydro-5H-benzo[b]carbazole-3-carbonitrile |
| | 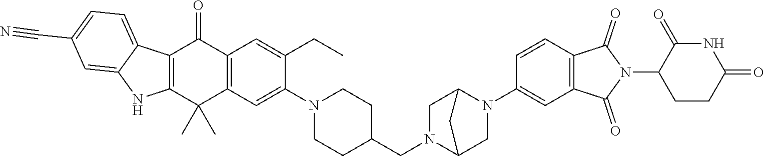 [SMILES: CCc1cc2c(cc1N1CCC(CN3CC4CC3CN4c3ccc4c(c3)C(=O)N(C3CCC(=O)NC3=O)C4=O)CC1)C(C)(C)c1[nH]c3cc(C#N)ccc3c1C2=O] | 8-(4-((5-(2-(2,6-dioxopiperidin-3-yl)-1,3- dioxoisoindolin-5-yl)-2,5- diazabicyclo[2.2.1]heptan-2- yl)methyl)piperidin-1-yl)-9-ethyl-6,6-dimethyl- 11-oxo-6,11-dihydro-5H-benzo[b]carbazole-3- carbonitrile |
| | 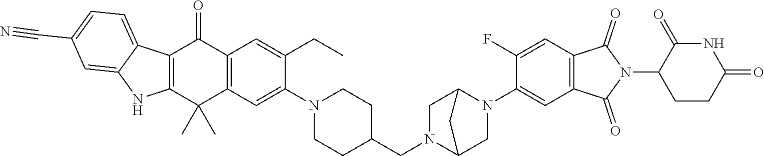 [SMILES: CCc1cc2c(cc1N1CCC(CN3CC4CC3CN4c3cc4c(cc3F)C(=O)N(C3CCC(=O)NC3=O)C4=O)CC1)C(C)(C)c1[nH]c3cc(C#N)ccc3c1C2=O] | 8-(4-((5-(2-(2,6-dioxopiperidin-3-yl)-6-fluoro- 1,3-dioxoisoindolin-5-yl)-2,5- diazabicyclo[2.2.1]heptan-2- yl)methyl)piperidin-1-yl)-9-ethyl-6,6-dimethyl- 11-oxo-6,11-dihydro-5H-benzo[b]carbazole-3- carbonitrile |
| | 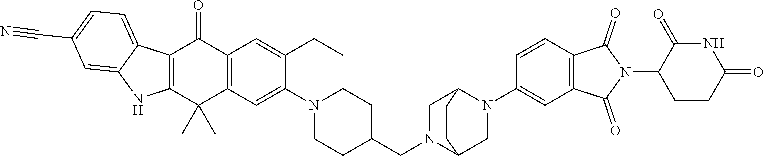 [SMILES: CCc1cc2c(cc1N1CCC(CN3CC4CCC3CN4c3ccc4c(c3)C(=O)N(C3CCC(=O)NC3=O)C4=O)CC1)C(C)(C)c1[nH]c3cc(C#N)ccc3c1C2=O] | 8-(4-((5-(2-(2,6-dioxopiperidin-3-yl)-1,3- dioxoisoindolin-5-yl)-2,5- diazabicyclo[2.2.2]octan-2-yl)methyl)piperidin- 1-yl)-9-ethyl-6,6-dimethyl-11-oxo-6,11- dihydro-5H-benzo[b]carbazole-3-carbonitrile |
| | 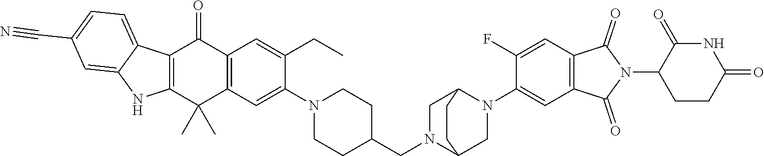 [SMILES: CCc1cc2c(cc1N1CCC(CN3CC4CCC3CN4c3cc4c(cc3F)C(=O)N(C3CCC(=O)NC3=O)C4=O)CC1)C(C)(C)c1[nH]c3cc(C#N)ccc3c1C2=O] | 8-(4-((5-(2-(2,6-dioxopiperidin-3-yl)-6-fluoro- 1,3-dioxoisoindolin-5-yl)-2,5- diazabicyclo[2.2.2]octan-2-yl)methyl)piperidin- 1-yl)-9-ethyl-6,6-dimethyl-11-oxo-6,11- dihydro-5H-benzo[b]carbazole-3-carbonitrile |
| | 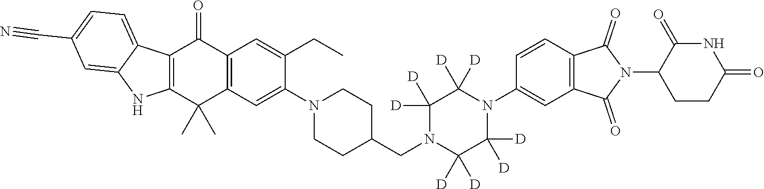 [SMILES: [2H]C1([2H])N(CC2CCN(c3cc4c(cc3CC)C(=O)c3c([nH]c5cc(C#N)ccc35)C4(C)C)CC2)C([2H])([2H])C([2H])([2H])N(c2ccc3c(c2)C(=O)N(C2CCC(=O)NC2=O)C3=O)C1([2H])[2H]] | 8-(4-((4-(2-(2,6-dioxopiperidin-3-yl)-1,3- dioxoisoindolin-5-yl)piperazin-1-yl- 2,2,3,3,5,5,6,6-d₈)methyl)piperidin-1-yl)-9- ethyl-6,6-dimethyl-11-oxo-6,11-dihydro-5H- benzo[b]carbazole-3-carbonitrile |
| | 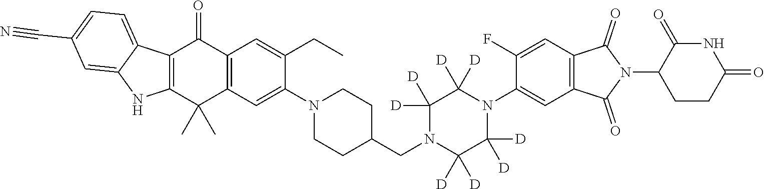 [SMILES: [2H]C1([2H])N(CC2CCN(c3cc4c(cc3CC)C(=O)c3c([nH]c5cc(C#N)ccc35)C4(C)C)CC2)C([2H])([2H])C([2H])([2H])N(c2cc3c(cc2F)C(=O)N(C2CCC(=O)NC2=O)C3=O)C1([2H])[2H]] | 8-(4-((4-(2-(2,6-dioxopiperidin-3-yl)-6-fluoro- 1,3-dioxoisoindolin-5-yl)piperazin-1-yl- 2,2,3,3,5,5,6,6-d₈)methyl)piperidin-1-yl)-9- ethyl-6,6-dimethyl-11-oxo-6,11-dihydro-5H- benzo[b]carbazole-3-carbonitrile |
| | 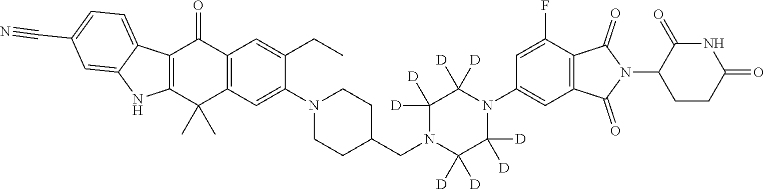 [SMILES: [2H]C1([2H])N(CC2CCN(c3cc4c(cc3CC)C(=O)c3c([nH]c5cc(C#N)ccc35)C4(C)C)CC2)C([2H])([2H])C([2H])([2H])N(c2cc(F)c3c(c2)C(=O)N(C2CCC(=O)NC2=O)C3=O)C1([2H])[2H]] | 8-(4-((4-(2-(2,6-dioxopiperidin-3-yl)-7-fluoro- 1,3-dioxoisoindolin-5-yl)piperazin-1-yl- 2,2,3,3,5,5,6,6-d₈)methyl)piperidin-1-yl)-9- ethyl-6,6-dimethyl-11-oxo-6,11-dihydro-5H- benzo[b]carbazole-3-carbonitrile |
| | 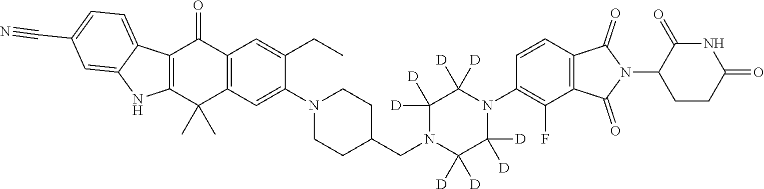 [SMILES: [2H]C1([2H])N(CC2CCN(c3cc4c(cc3CC)C(=O)c3c([nH]c5cc(C#N)ccc35)C4(C)C)CC2)C([2H])([2H])C([2H])([2H])N(c2ccc3c(c2F)C(=O)N(C2CCC(=O)NC2=O)C3=O)C1([2H])[2H]] | 8-(4-((4-(2-(2,6-dioxopiperidin-3-yl)-4-fluoro- 1,3-dioxoisoindolin-5-yl)piperazin-1-yl- 2,2,3,3,5,5,6,6-d₈)methyl)piperidin-1-yl)-9- ethyl-6,6-dimethyl-11-oxo-6,11-dihydro-5H- benzo[b]carbazole-3-carbonitrile |
| | 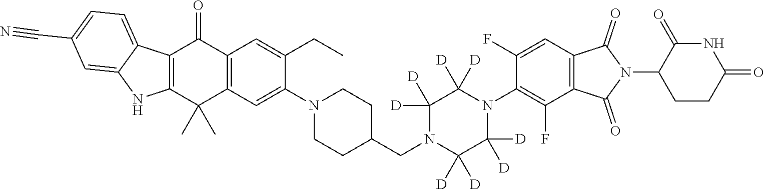 [SMILES: [2H]C1([2H])N(CC2CCN(c3cc4c(cc3CC)C(=O)c3c([nH]c5cc(C#N)ccc35)C4(C)C)CC2)C([2H])([2H])C([2H])([2H])N(c2c(F)cc3c(c2F)C(=O)N(C2CCC(=O)NC2=O)C3=O)C1([2H])[2H]] | 8-(4-((4-(2-(2,6-dioxopiperidin-3-yl)-4,6- difluoro-1,3-dioxoisoindolin-5-yl)piperazin-1- yl-2,2,3,3,5,5,6,6-d₈)methyl)piperidin-1-yl)-9- ethyl-6,6-dimethyl-11-oxo-6,11-dihydro-5H- benzo[b]carbazole-3-carbonitrile |
| | 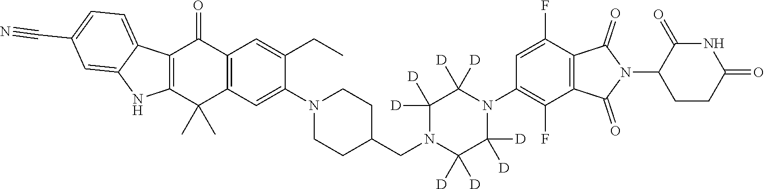 [SMILES: [2H]C1([2H])N(CC2CCN(c3cc4c(cc3CC)C(=O)c3c([nH]c5cc(C#N)ccc35)C4(C)C)CC2)C([2H])([2H])C([2H])([2H])N(c2cc(F)c3c(c2F)C(=O)N(C2CCC(=O)NC2=O)C3=O)C1([2H])[2H]] | 8-(4-((4-(2-(2,6-dioxopiperidin-3-yl)-4,7- difluoro-1,3-dioxoisoindolin-5-yl)piperazin-1- yl-2,2,3,3,5,5,6,6-d₈)methyl)piperidin-1-yl)-9- ethyl-6,6-dimethyl-11-oxo-6,11-dihydro-5H- benzo[b]carbazole-3-carbonitrile |
| | 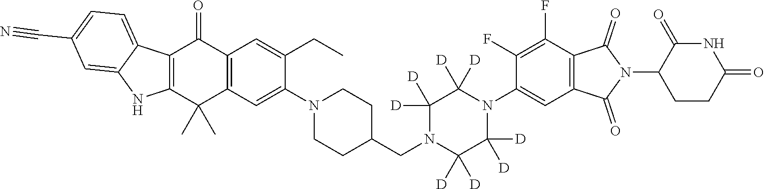 [SMILES: [2H]C1([2H])N(CC2CCN(c3cc4c(cc3CC)C(=O)c3c([nH]c5cc(C#N)ccc35)C4(C)C)CC2)C([2H])([2H])C([2H])([2H])N(c2cc3c(c(F)c2F)C(=O)N(C2CCC(=O)NC2=O)C3=O)C1([2H])[2H]] | 8-(4-((4-(2-(2,6-dioxopiperidin-3-yl)-6,7- difluoro-1,3-dioxoisoindolin-5-yl)piperazin-1- yl-2,2,3,3,5,5,6,6-d₈)methyl)piperidin-1-yl)-9- ethyl-6,6-dimethyl-11-oxo-6,11-dihydro-5H- benzo[b]carbazole-3-carbonitrile |
| | 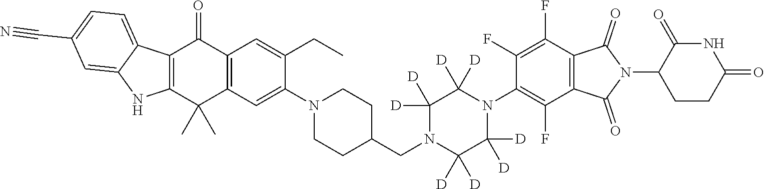 [SMILES: [2H]C1([2H])N(CC2CCN(c3cc4c(cc3CC)C(=O)c3c([nH]c5cc(C#N)ccc35)C4(C)C)CC2)C([2H])([2H])C([2H])([2H])N(c2c(F)c(F)c3c(c2F)C(=O)N(C2CCC(=O)NC2=O)C3=O)C1([2H])[2H]] | 8-(4-((4-(2-(2,6-dioxopiperidin-3-yl)-4,6,7- trifluoro-1,3-dioxoisoindolin-5-yl)piperazin-1- yl-2,2,3,3,5,5,6,6-d₈)methyl)piperidin-1-yl)-9- ethyl-6,6-dimethyl-11-oxo-6,11-dihydro-5H- benzo[b]carbazole-3-carbonitrile |
| | 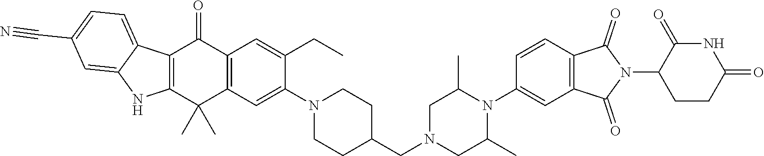 [SMILES: CCc1cc2c(cc1N1CCC(CN3CC(C)N(c4ccc5c(c4)C(=O)N(C4CCC(=O)NC4=O)C5=O)C(C)C3)CC1)C(C)(C)c1[nH]c3cc(C#N)ccc3c1C2=O] | 8-(4-((4-(2-(2,6-dioxopiperidin-3-yl)-1,3- dioxoisoindolin-5-yl)-3,5-dimethylpiperazin-1- yl)methyl)piperidin-1-yl)-9-ethyl-6,6-dimethyl- 11-oxo-6,11-dihydro-5H-benzo[b]carbazole-3- carbonitrile |
| | 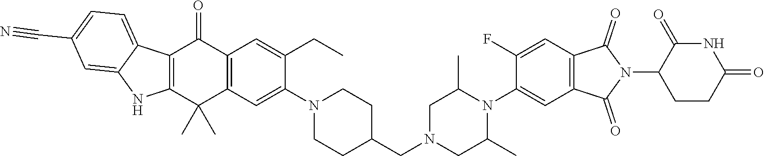 [SMILES: CCc1cc2c(cc1N1CCC(CN3CC(C)N(c4cc5c(cc4F)C(=O)N(C4CCC(=O)NC4=O)C5=O)C(C)C3)CC1)C(C)(C)c1[nH]c3cc(C#N)ccc3c1C2=O] | 8-(4-((4-(2-(2,6-dioxopiperidin-3-yl)-6-fluoro- 1,3-dioxoisoindolin-5-yl)-3,5- dimethylpiperazin-1-yl)methyl)piperidin-1-yl)- 9-ethyl-6,6-dimethyl-11-oxo-6,11-dihydro-5H- benzo[b]carbazole-3-carbonitrile |
| | 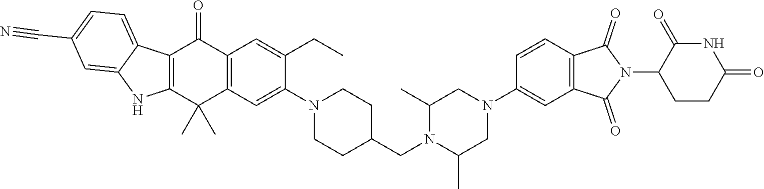 [SMILES: CCc1cc2c(cc1N1CCC(CN3C(C)CN(c4ccc5c(c4)C(=O)N(C4CCC(=O)NC4=O)C5=O)CC3C)CC1)C(C)(C)c1[nH]c3cc(C#N)ccc3c1C2=O] | 8-(4-((4-(2-(2,6-dioxopiperidin-3-yl)-1,3- dioxoisoindolin-5-yl)-2,6-dimethylpiperazin-1- yl)methyl)piperidin-1-yl)-9-ethyl-6,6-dimethyl- 11-oxo-6,11-dihydro-5H-benzo[b]carbazole-3- carbonitrile |
| | 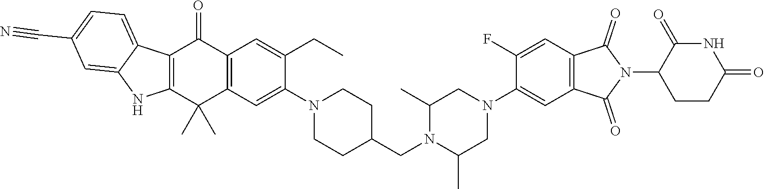 [SMILES: CCc1cc2c(cc1N1CCC(CN3C(C)CN(c4cc5c(cc4F)C(=O)N(C4CCC(=O)NC4=O)C5=O)CC3C)CC1)C(C)(C)c1[nH]c3cc(C#N)ccc3c1C2=O] | 8-(4-((4-(2-(2,6-dioxopiperidin-3-yl)-6-fluoro- 1,3-dioxoisoindolin-5-yl)-2,6- dimethylpiperazin-1-yl)methyl)piperidin-1-yl)- 9-ethyl-6,6-dimethyl-11-oxo-6,11-dihydro-5H- benzo[b]carbazole-3-carbonitrile |
| | 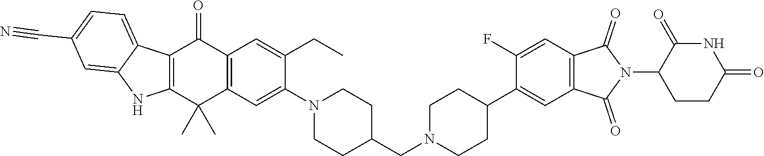 [SMILES: CCc1cc2c(cc1N1CCC(CN3CCC(c4cc5c(cc4F)C(=O)N(C4CCC(=O)NC4=O)C5=O)CC3)CC1)C(C)(C)c1[nH]c3cc(C#N)ccc3c1C2=O] | 8-(4-((4-(2-(2,6-dioxopiperidin-3-yl)-6-fluoro- 1,3-dioxoisoindolin-5-yl)piperidin-1- yl)methyl)piperidin-1-yl)-9-ethyl-6,6-dimethyl- 11-oxo-6,11-dihydro-5H-benzo[b]carbazole-3- carbonitrile |
| | 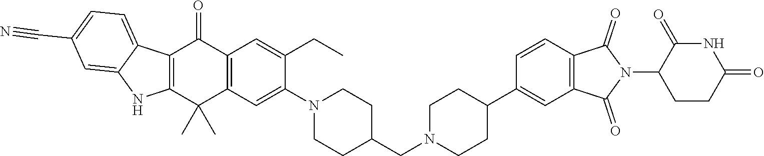 [SMILES: CCc1cc2c(cc1N1CCC(CN3CCC(c4ccc5c(c4)C(=O)N(C4CCC(=O)NC4=O)C5=O)CC3)CC1)C(C)(C)c1[nH]c3cc(C#N)ccc3c1C2=O] | 8-(4-((4-(2-(2,6-dioxopiperidin-3-yl)-1,3- dioxoisoindolin-5-yl)piperidin-1- yl)methyl)piperidin-1-yl)-9-ethyl-6,6-dimethyl- 11-oxo-6,11-dihydro-5H-benzo[b]carbazole-3- carbonitrile |
| | 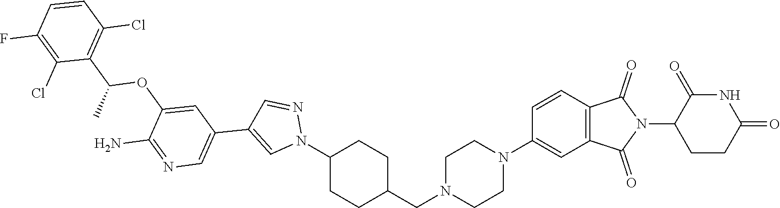 [SMILES: C[C@@H](Oc1cc(-c2cnn(C3CCC(CN4CCN(c5ccc6c(c5)C(=O)N(C5CCC(=O)NC5=O)C6=O)CC4)CC3)c2)cnc1N)c1c(Cl)ccc(F)c1Cl] | 5-(4-((4-(4-(6-amino-5-((R)-1-(2,6-dichloro-3- fluorophenyl)ethoxy)pyridin-3-yl)-1H-pyrazol- 1-yl)cyclohexyl)methyl)piperazin-1-yl)-2-(2,6- dioxopiperidin-3-yl)isoindoline-1,3-dione |
| | 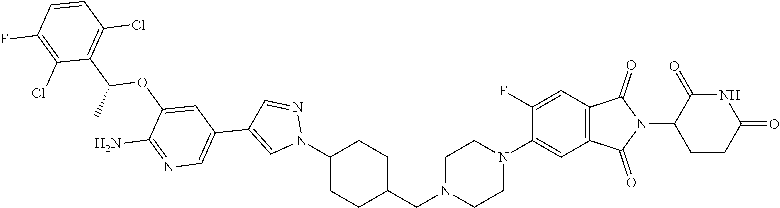 [SMILES: C[C@@H](Oc1cc(-c2cnn(C3CCC(CN4CCN(c5cc6c(cc5F)C(=O)N(C5CCC(=O)NC5=O)C6=O)CC4)CC3)c2)cnc1N)c1c(Cl)ccc(F)c1Cl] | 5-(4-((4-(4-(6-amino-5-((R)-1-(2,6-dichloro-3- fluorophenyl)ethoxy)pyridin-3-yl)-1H-pyrazol- 1-yl)cyclohexyl)methyl)piperazin-1-yl)-2-(2,6- dioxopiperidin-3-yl)-6-fluoroisoindoline-1,3- dione |
| | 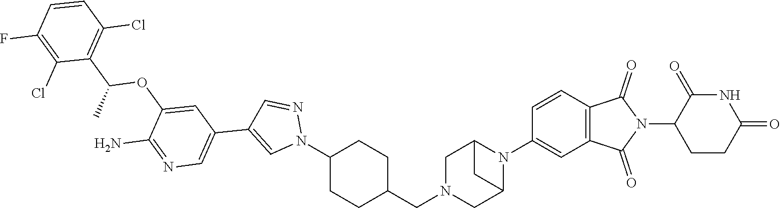 [SMILES: C[C@@H](Oc1cc(-c2cnn(C3CCC(CN4CC5CC(C4)N5c4ccc5c(c4)C(=O)N(C4CCC(=O)NC4=O)C5=O)CC3)c2)cnc1N)c1c(Cl)ccc(F)c1Cl] | 5-(3-((4-(4-(6-amino-5-((R)-1-(2,6-dichloro-3- fluorophenyl)ethoxy)pyridin-3-yl)-1H-pyrazol- 1-yl)cyclohexyl)methyl)-3,6- diazabicyclo[3.1.1]heptan-6-yl)-2-(2,6- dioxopiperidin-3-yl)isoindoline-1,3-dione |
| | 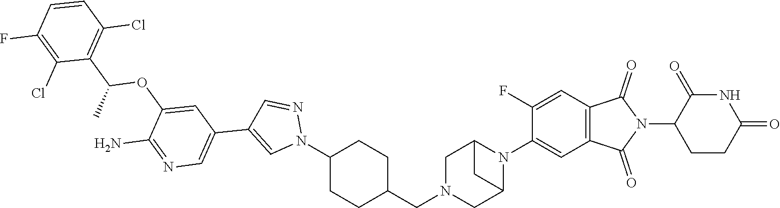 [SMILES: C[C@@H](Oc1cc(-c2cnn(C3CCC(CN4CC5CC(C4)N5c4cc5c(cc4F)C(=O)N(C4CCC(=O)NC4=O)C5=O)CC3)c2)cnc1N)c1c(Cl)ccc(F)c1Cl] | 5-(3-((4-(4-(6-amino-5-((R)-1-(2,6-dichloro-3- fluorophenyl)ethoxy)pyridin-3-yl)-1H-pyrazol- 1-yl)cyclohexyl)methyl)-3,6- diazabicyclo[3.1.1]heptan-6-yl)-2-(2,6- dioxopiperidin-3-yl)-6-fluoroisoindoline-1,3- dione |
| | 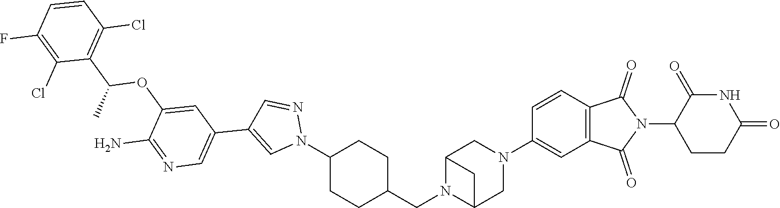 [SMILES: C[C@@H](Oc1cc(-c2cnn(C3CCC(CN4C5CC4CN(c4ccc6c(c4)C(=O)N(C4CCC(=O)NC4=O)C6=O)C5)CC3)c2)cnc1N)c1c(Cl)ccc(F)c1Cl] | 5-(6-((4-(4-(6-amino-5-((R)-1-(2,6-dichloro-3- fluorophenyl)ethoxy)pyridin-3-yl)-1H-pyrazol- 1-yl)cyclohexyl)methyl)-3,6- diazabicyclo[3.1.1]heptan-3-yl)-2-(2,6- dioxopiperidin-3-yl)isoindoline-1,3-dione |
| | 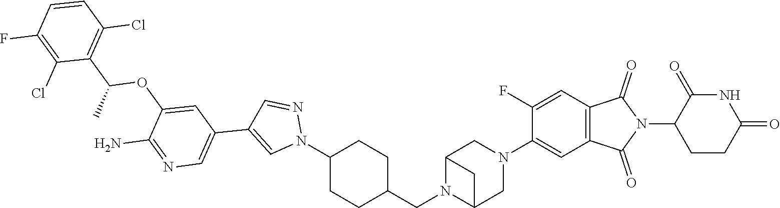 [SMILES: C[C@@H](Oc1cc(-c2cnn(C3CCC(CN4C5CC4CN(c4cc6c(cc4F)C(=O)N(C4CCC(=O)NC4=O)C6=O)C5)CC3)c2)cnc1N)c1c(Cl)ccc(F)c1Cl] | 5-(6-((4-(4-(6-amino-5-((R)-1-(2,6-dichloro-3- fluorophenyl)ethoxy)pyridin-3-yl)-1H-pyrazol- 1-yl)cyclohexyl)methyl)-3,6- diazabicyclo[3.1.1]heptan-3-yl)-2-(2,6- dioxopiperidin-3-yl)-6-fluoroisoindoline-1,3- dione |
| | 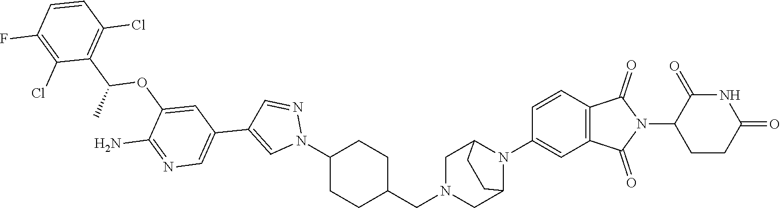 [SMILES: C[C@@H](Oc1cc(-c2cnn(C3CCC(CN4CC5CCC(C4)N5c4ccc5c(c4)C(=O)N(C4CCC(=O)NC4=O)C5=O)CC3)c2)cnc1N)c1c(Cl)ccc(F)c1Cl] | 5-(3-((4-(4-(6-amino-5-((R)-1-(2,6-dichloro-3- fluorophenyl)ethoxy)pyridin-3-yl)-1H-pyrazol- 1-yl)cyclohexyl)methyl)-3,8- diazabicyclo[3.2.1]octan-8-yl)-2-(2,6- dioxopiperidin-3-yl)isoindoline-1,3-dione |
| | 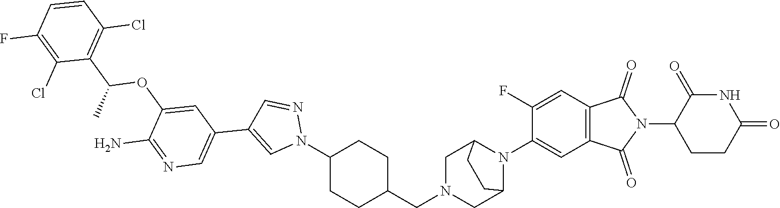 [SMILES: C[C@@H](Oc1cc(-c2cnn(C3CCC(CN4CC5CCC(C4)N5c4cc5c(cc4F)C(=O)N(C4CCC(=O)NC4=O)C5=O)CC3)c2)cnc1N)c1c(Cl)ccc(F)c1Cl] | 5-(3-((4-(4-(6-amino-5-((R)-1-(2,6-dichloro-3- fluorophenyl)ethoxy)pyridin-3-yl)-1H-pyrazol- 1-yl)cyclohexyl)methyl)-3,8- diazabicyclo[3.2.1]octan-8-yl)-2-(2,6- dioxopiperidin-3-yl)-6-fluoroisoindoline-1,3- dione |
| | 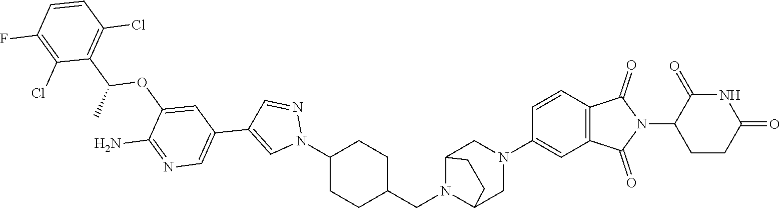 [SMILES: C[C@@H](Oc1cc(-c2cnn(C3CCC(CN4C5CCC4CN(c4ccc6c(c4)C(=O)N(C4CCC(=O)NC4=O)C6=O)C5)CC3)c2)cnc1N)c1c(Cl)ccc(F)c1Cl] | 5-(8-((4-(4-(6-amino-5-((R)-1-(2,6-dichloro-3- fluorophenyl)ethoxy)pyridin-3-yl)-1H-pyrazol- 1-yl)cyclohexyl)methyl)-3,8- diazabicyclo[3.2.1]octan-3-yl)-2-(2,6- dioxopiperidin-3-yl)isoindoline-1,3-dione |
| | 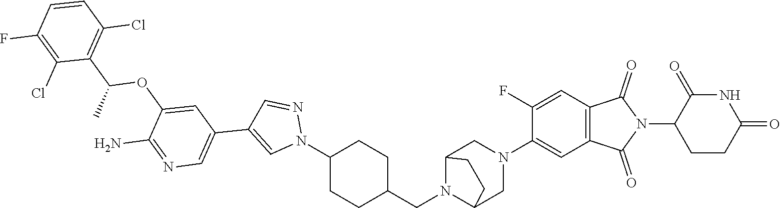 [SMILES: C[C@@H](Oc1cc(-c2cnn(C3CCC(CN4C5CCC4CN(c4cc6c(cc4F)C(=O)N(C4CCC(=O)NC4=O)C6=O)C5)CC3)c2)cnc1N)c1c(Cl)ccc(F)c1Cl] | 5-(8-((4-(4-(6-amino-5-((R)-1-(2,6-dichloro-3- fluorophenyl)ethoxy)pyridin-3-yl)-1H-pyrazol- 1-yl)cyclohexyl)methyl)-3,8- diazabicyclo[3.2.1]octan-3-yl)-2-(2,6- dioxopiperidin-3-yl)-6-fluoroisoindoline-1,3- dione |
| | 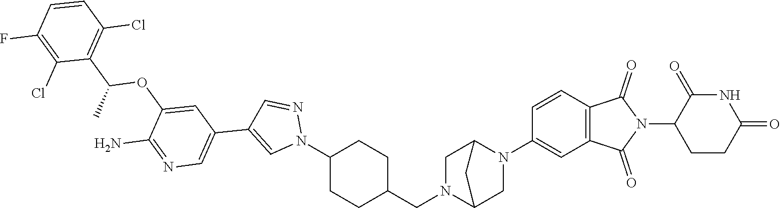 [SMILES: C[C@@H](Oc1cc(-c2cnn(C3CCC(CN4CC5CC4CN5c4ccc5c(c4)C(=O)N(C4CCC(=O)NC4=O)C5=O)CC3)c2)cnc1N)c1c(Cl)ccc(F)c1Cl] | 5-(5-((4-(4-(6-amino-5-((R)-1-(2,6-dichloro-3- fluorophenyl)ethoxy)pyridin-3-yl)-1H-pyrazol- 1-yl)cyclohexyl)methyl)-2,5- diazabicyclo[2.2.1]heptan-2-yl)-2-(2,6- dioxopiperidin-3-yl)isoindoline-1,3-dione |
| | 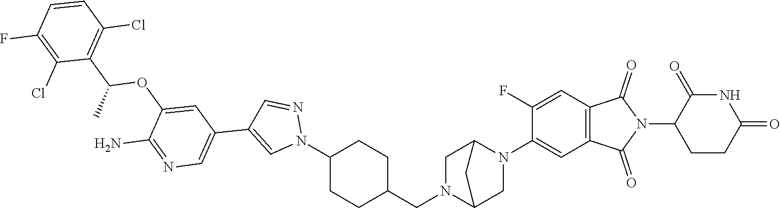 [SMILES: C[C@@H](Oc1cc(-c2cnn(C3CCC(CN4CC5CC4CN5c4cc5c(cc4F)C(=O)N(C4CCC(=O)NC4=O)C5=O)CC3)c2)cnc1N)c1c(Cl)ccc(F)c1Cl] | 5-(5-((4-(4-(6-amino-5-((R)-1-(2,6-dichloro-3- fluorophenyl)ethoxy)pyridin-3-yl)-1H-pyrazol- 1-yl)cyclohexyl)methyl)-2,5- diazabicyclo[2.2.1]heptan-2-yl)-2-(2,6- dioxopiperidin-3-yl)-6-fluoroisoindoline-1,3- dione |
| | 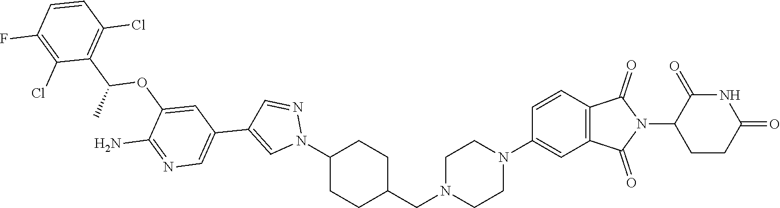 [SMILES: C[C@@H](Oc1cc(-c2cnn(C3CCC(CN4CCN(c5ccc6c(c5)C(=O)N(C5CCC(=O)NC5=O)C6=O)CC4)CC3)c2)cnc1N)c1c(Cl)ccc(F)c1Cl] | 5-(4-((4-(4-(6-amino-5-((R)-1-(2,6-dichloro-3- fluorophenyl)ethoxy)pyridin-3-yl)-1H-pyrazol- 1-yl)cyclohexyl)methyl)piperazin-1-yl)-2-(2,6- dioxopiperidin-3-yl)isoindoline-1,3-dione |
| | 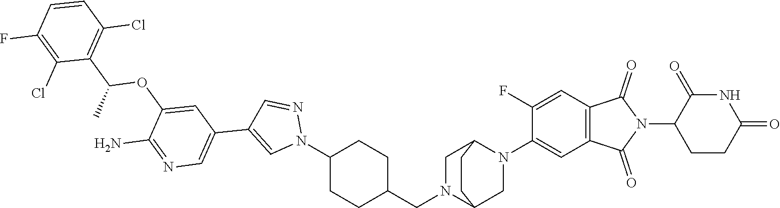 [SMILES: C[C@@H](Oc1cc(-c2cnn(C3CCC(CN4CC5CCC4CN5c4cc5c(cc4F)C(=O)N(C4CCC(=O)NC4=O)C5=O)CC3)c2)cnc1N)c1c(Cl)ccc(F)c1Cl] | 5-(5-((4-(4-(6-amino-5-((R)-1-(2,6-dichloro-3- fluorophenyl)ethoxy)pyridin-3-yl)-1H-pyrazol- 1-yl)cyclohexyl)methyl)-2,5- diazabicyclo[2.2.2]octan-2-yl)-2-(2,6- dioxopiperidin-3-yl)-6-fluoroisoindoline-1,3- dione |
| | 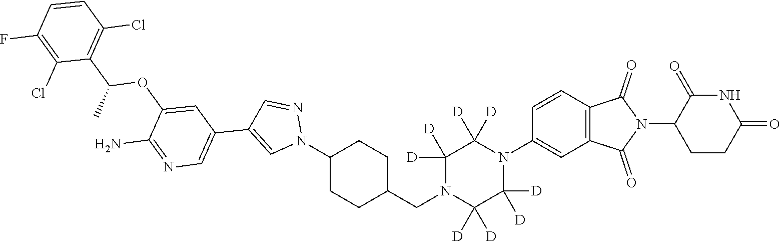 [SMILES: [2H]C1([2H])N(CC2CCC(n3cc(-c4cnc(N)c(O[C@H](C)c5c(Cl)ccc(F)c5Cl)c4)cn3)CC2)C([2H])([2H])C([2H])([2H])N(c2ccc3c(c2)C(=O)N(C2CCC(=O)NC2=O)C3=O)C1([2H])[2H]] | 5-(4-((4-(4-(6-amino-5-((R)-1-(2,6-dichloro-3- fluorophenyl)ethoxy)pyridin-3-yl)-1H-pyrazol- 1-yl)cyclohexyl)methyl)piperazin-1-yl- 2,2,3,3,5,5,6,6-d₈)-2-(2,6-dioxopiperidin-3- yl)isoindoline-1,3-dione |
| | 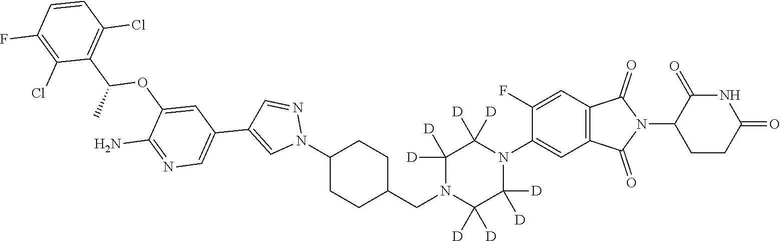 [SMILES: [2H]C1([2H])N(CC2CCC(n3cc(-c4cnc(N)c(O[C@H](C)c5c(Cl)ccc(F)c5Cl)c4)cn3)CC2)C([2H])([2H])C([2H])([2H])N(c2cc3c(cc2F)C(=O)N(C2CCC(=O)NC2=O)C3=O)C1([2H])[2H]] | 5-(4-((4-(4-(6-amino-5-((R)-1-(2,6-dichloro-3- fluorophenyl)ethoxy)pyridin-3-yl)-1H-pyrazol- 1-yl)cyclohexyl)methyl)piperazin-1-yl- 2,2,3,3,5,5,6,6-d₈)-2-(2,6-dioxopiperidin-3-yl)- 6-fluoroisoindoline-1,3-dione |
| | 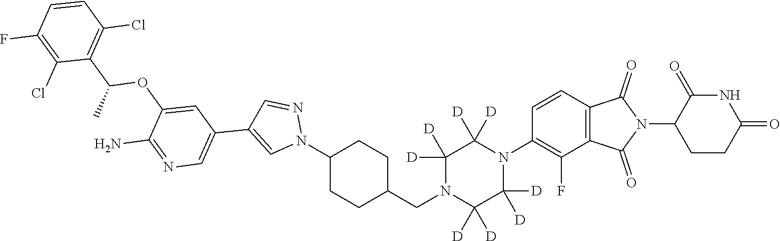 [SMILES: [2H]C1([2H])N(CC2CCC(n3cc(-c4cnc(N)c(O[C@H](C)c5c(Cl)ccc(F)c5Cl)c4)cn3)CC2)C([2H])([2H])C([2H])([2H])N(c2ccc3c(c2F)C(=O)N(C2CCC(=O)NC2=O)C3=O)C1([2H])[2H]] | 5-(4-((4-(4-(6-amino-5-((R)-1-(2,6-dichloro-3- fluorophenyl)ethoxy)pyridin-3-yl)-1H-pyrazol- 1-yl)cyclohexyl)methyl)piperazin-1-yl- 2,2,3,3,5,5,6,6-d₈)-2-(2,6-dioxopiperidin-3-yl)- 4-fluoroisoindoline-1,3-dione |
| | 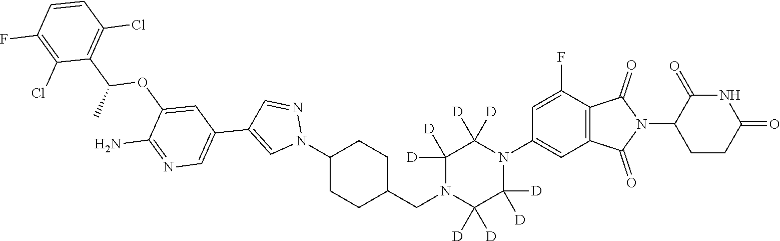 [SMILES: [2H]C1([2H])N(CC2CCC(n3cc(-c4cnc(N)c(O[C@H](C)c5c(Cl)ccc(F)c5Cl)c4)cn3)CC2)C([2H])([2H])C([2H])([2H])N(c2cc(F)c3c(c2)C(=O)N(C2CCC(=O)NC2=O)C3=O)C1([2H])[2H]] | 6-(4-((4-(4-(6-amino-5-((R)-1-(2,6-dichloro-3- fluorophenyl)ethoxy)pyridin-3-yl)-1H-pyrazol- 1-yl)cyclohexyl)methyl)piperazin-1-yl- 2,2,3,3,5,5,6,6-d₈)-2-(2,6-dioxopiperidin-3-yl)- 4-fluoroisoindoline-1,3-dione |
| | 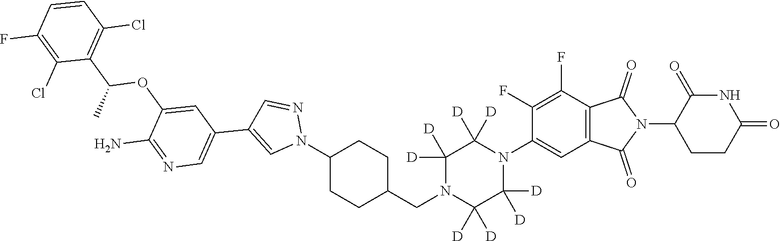 [SMILES: [2H]C1([2H])N(CC2CCC(n3cc(-c4cnc(N)c(O[C@H](C)c5c(Cl)ccc(F)c5Cl)c4)cn3)CC2)C([2H])([2H])C([2H])([2H])N(c2cc3c(c(F)c2F)C(=O)N(C2CCC(=O)NC2=O)C3=O)C1([2H])[2H]] | 6-(4-((4-(4-(6-amino-5-((R)-1-(2,6-dichloro-3- fluorophenyl)ethoxy)pyridin-3-yl)-1H-pyrazol- 1-yl)cyclohexyl)methyl)piperazin-1-yl- 2,2,3,3,5,5,6,6-d₈)-2-(2,6-dioxopiperidin-3-yl)- 4,5-difluoroisoindoline-1,3-dione |
| | 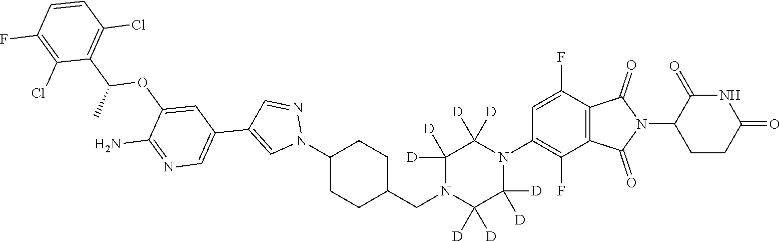 [SMILES: [2H]C1([2H])N(CC2CCC(n3cc(-c4cnc(N)c(O[C@H](C)c5c(Cl)ccc(F)c5Cl)c4)cn3)CC2)C([2H])([2H])C([2H])([2H])N(c2cc(F)c3c(c2F)C(=O)N(C2CCC(=O)NC2=O)C3=O)C1([2H])[2H]] | 5-(4-((4-(4-(6-amino-5-((R)-1-(2,6-dichloro-3- fluorophenyl)ethoxy)pyridin-3-yl)-1H-pyrazol- 1-yl)cyclohexyl)methyl)piperazin-1-yl- 2,2,3,3,5,5,6,6-d₈)-2-(2,6-dioxopiperidin-3-yl)- 4,7-difluoroisoindoline-1,3-dione |
| | 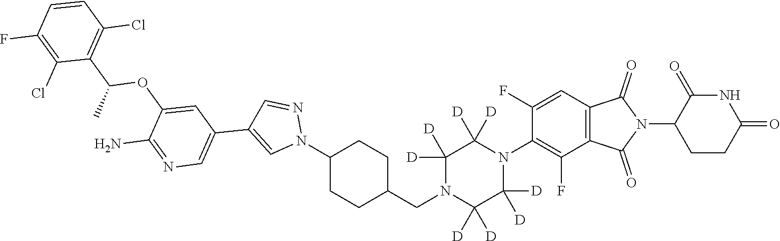 [SMILES: [2H]C1([2H])N(CC2CCC(n3cc(-c4cnc(N)c(O[C@H](C)c5c(Cl)ccc(F)c5Cl)c4)cn3)CC2)C([2H])([2H])C([2H])([2H])N(c2c(F)cc3c(c2F)C(=O)N(C2CCC(=O)NC2=O)C3=O)C1([2H])[2H]] | 5-(4-((4-(4-(6-amino-5-((R)-1-(2,6-dichloro-3- fluorophenyl)ethoxy)pyridin-3-yl)-1H-pyrazol- 1-yl)cyclohexyl)methyl)piperazin-1-yl- 2,2,3,3,5,5,6,6-d₈)-2-(2,6-dioxopiperidin-3-yl)- 4,6-difluoroisoindoline-1,3-dione |
| | 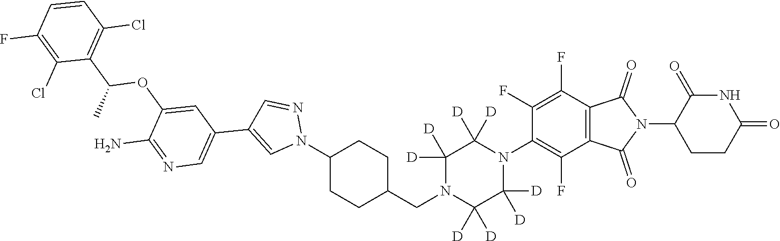 [SMILES: [2H]C1([2H])N(CC2CCC(n3cc(-c4cnc(N)c(O[C@H](C)c5c(Cl)ccc(F)c5Cl)c4)cn3)CC2)C([2H])([2H])C([2H])([2H])N(c2c(F)c(F)c3c(c2F)C(=O)N(C2CCC(=O)NC2=O)C3=O)C1([2H])[2H]] | 5-(4-((4-(4-(6-amino-5-((R)-1-(2,6-dichloro-3- fluorophenyl)ethoxy)pyridin-3-yl)-1H-pyrazol- 1-yl)cyclohexyl)methyl)piperazin-1-yl- 2,2,3,3,5,5,6,6-d₈)-2-(2,6-dioxopiperidin-3-yl)- 4,6,7-trifluoroisoindoline-1,3-dione |
| | 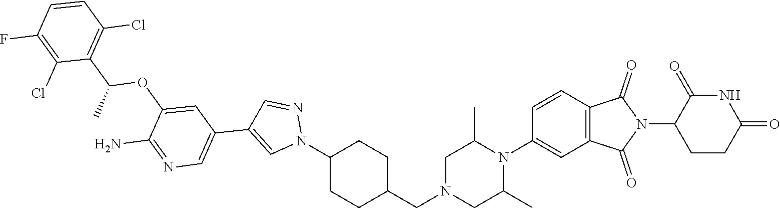 [SMILES: CC1CN(CC2CCC(n3cc(-c4cnc(N)c(O[C@H](C)c5c(Cl)ccc(F)c5Cl)c4)cn3)CC2)CC(C)N1c1ccc2c(c1)C(=O)N(C1CCC(=O)NC1=O)C2=O] | 5-(4-((4-(4-(6-amino-5-((R)-1-(2,6-dichloro-3- fluorophenyl)ethoxy)pyridin-3-yl)-1H-pyrazol- 1-yl)cyclohexyl)methyl)-2,6- dimethylpiperazin-1-yl)-2-(2,6-dioxopiperidin- 3-yl)isoindoline-1,3-dione |
| | 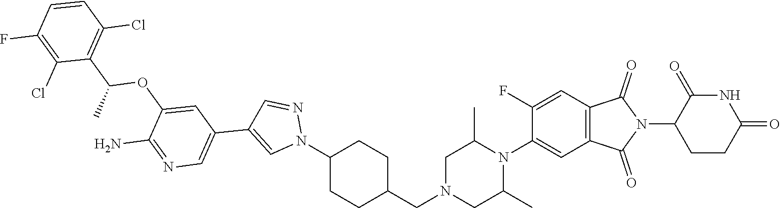 [SMILES: CC1CN(CC2CCC(n3cc(-c4cnc(N)c(O[C@H](C)c5c(Cl)ccc(F)c5Cl)c4)cn3)CC2)CC(C)N1c1cc2c(cc1F)C(=O)N(C1CCC(=O)NC1=O)C2=O] | 5-(4-((4-(4-(6-amino-5-((R)-1-(2,6-dichloro-3- fluorophenyl)ethoxy)pyridin-3-yl)-1H-pyrazol- 1-yl)cyclohexyl)methyl)-2,6- dimethylpiperazin-1-yl)-2-(2,6-dioxopiperidin- 3-yl)-6-fluoroisoindoline-1,3-dione |
| | 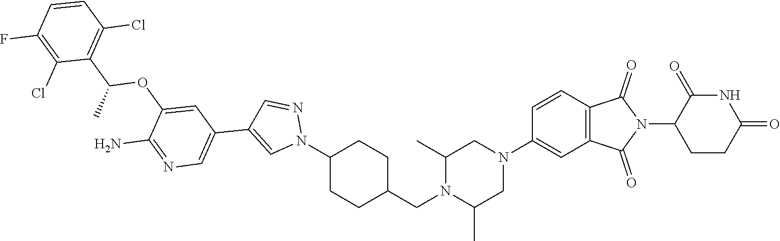 [SMILES: CC1CN(c2ccc3c(c2)C(=O)N(C2CCC(=O)NC2=O)C3=O)CC(C)N1CC1CCC(n2cc(-c3cnc(N)c(O[C@H](C)c4c(Cl)ccc(F)c4Cl)c3)cn2)CC1] | 5-(4-((4-(4-(6-amino-5-((R)-1-(2,6-dichloro-3- fluorophenyl)ethoxy)pyridin-3-yl)-1H-pyrazol- 1-yl)cyclohexyl)methyl)-3,5- dimethylpiperazin-1-yl)-2-(2,6-dioxopiperidin- 3-yl)isoindoline-1,3-dione |
| | 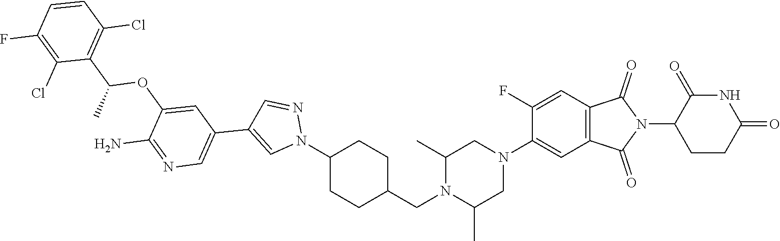 [SMILES: CC1CN(c2cc3c(cc2F)C(=O)N(C2CCC(=O)NC2=O)C3=O)CC(C)N1CC1CCC(n2cc(-c3cnc(N)c(O[C@H](C)c4c(Cl)ccc(F)c4Cl)c3)cn2)CC1] | 5-(4-((4-(4-(6-amino-5-((R)-1-(2,6-dichloro-3- fluorophenyl)ethoxy)pyridin-3-yl)-1H-pyrazol- 1-yl)cyclohexyl)methyl)-3,5- dimethylpiperazin-1-yl)-2-(2,6-dioxopiperidin- 3-yl)-6-fluoroisoindoline-1,3-dione |
| | 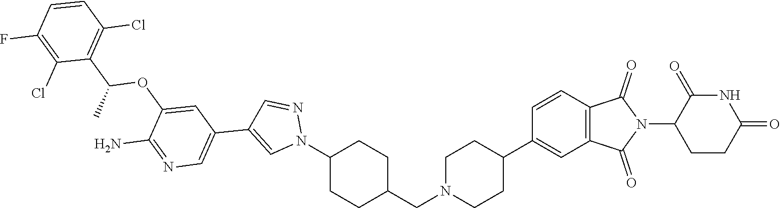 [SMILES: C[C@@H](Oc1cc(-c2cnn(C3CCC(CN4CCC(c5ccc6c(c5)C(=O)N(C5CCC(=O)NC5=O)C6=O)CC4)CC3)c2)cnc1N)c1c(Cl)ccc(F)c1Cl] | 5-(1-((4-(4-(6-amino-5-((R)-1-(2,6-dichloro-3- fluorophenyl)ethoxy)pyridin-3-yl)-1H-pyrazol- 1-yl)cyclohexyl)methyl)piperidin-4-yl)-2-(2,6- dioxopiperidin-3-yl)isoindoline-1,3-dione |
| | 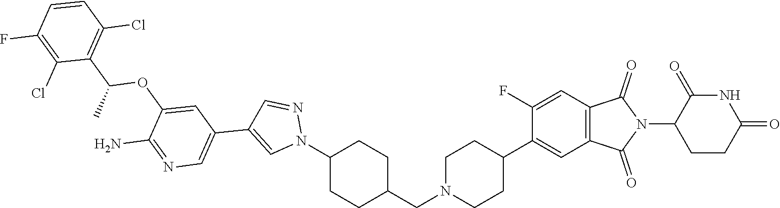 [SMILES: C[C@@H](Oc1cc(-c2cnn(C3CCC(CN4CCC(c5cc6c(cc5F)C(=O)N(C5CCC(=O)NC5=O)C6=O)CC4)CC3)c2)cnc1N)c1c(Cl)ccc(F)c1Cl] | 5-(1-((4-(4-(6-amino-5-((R)-1-(2,6-dichloro-3- fluorophenyl)ethoxy)pyridin-3-yl)-1H-pyrazol- 1-yl)cyclohexyl)methyl)piperidin-4-yl)-2-(2,6- dioxopiperidin-3-yl)-6-fluoroisoindoline-1,3- dione |
| | 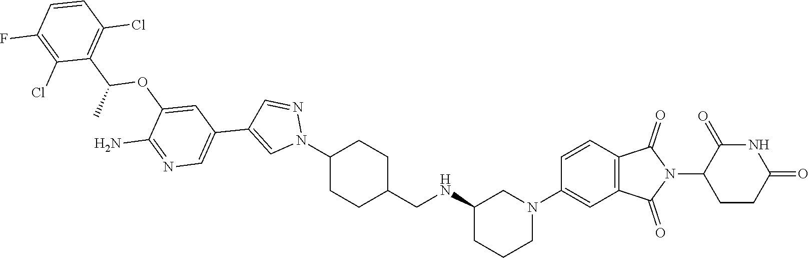 [SMILES: C[C@@H](Oc1cc(-c2cnn(C3CCC(CN[C@@H]4CCCN(c5ccc6c(c5)C(=O)N(C5CCC(=O)NC5=O)C6=O)C4)CC3)c2)cnc1N)c1c(Cl)ccc(F)c1Cl] | 5-((R)-3-(((4-(4-(6-amino-5-((R)-1-(2,6- dichloro-3-fluorophenyl)ethoxy)pyridin-3-yl)- 1H-pyrazol-1- yl)cyclohexyl)methyl)amino)piperidin-1-yl)-2- (2,6-dioxopiperidin-3-yl)isoindoline-1,3-dione |
| | 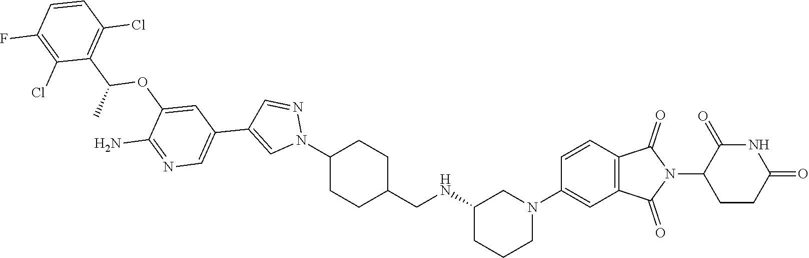 [SMILES: C[C@@H](Oc1cc(-c2cnn(C3CCC(CN[C@H]4CCCN(c5ccc6c(c5)C(=O)N(C5CCC(=O)NC5=O)C6=O)C4)CC3)c2)cnc1N)c1c(Cl)ccc(F)c1Cl] | 5-((S)-3-(((4-(4-(6-amino-5-((R)-1-(2,6- dichloro-3-fluorophenyl)ethoxy)pyridin-3-yl)- 1H-pyrazol-1- yl)cyclohexyl)methyl)amino)piperidin-1-yl)-2- (2,6-dioxopiperidin-3-yl)isoindoline-1,3-dione |
| | 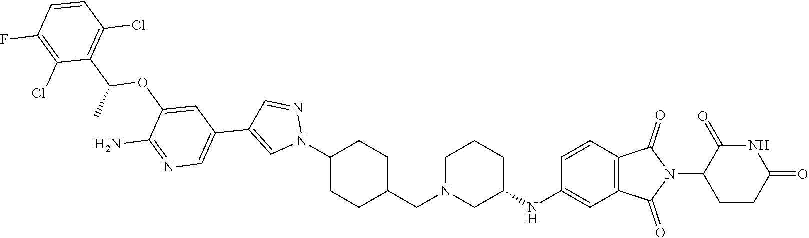 [SMILES: C[C@@H](Oc1cc(-c2cnn(C3CCC(CN4CCC[C@H](Nc5ccc6c(c5)C(=O)N(C5CCC(=O)NC5=O)C6=O)C4)CC3)c2)cnc1N)c1c(Cl)ccc(F)c1Cl] | 5-(((S)-1-((4-(4-(6-amino-5-((R)-1-(2,6- dichloro-3-fluorophenyl)ethoxy)pyridin-3-yl)- 1H-pyrazol-1-yl)cyclohexyl)methyl)piperidin- 3-yl)amino)-2-(2,6-dioxopiperidin-3- yl)isoindoline-1,3-dione |
| | 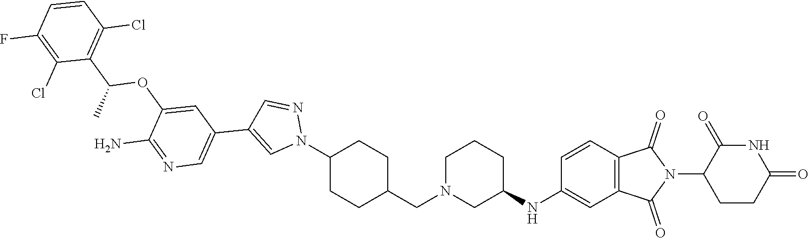 [SMILES: C[C@@H](Oc1cc(-c2cnn(C3CCC(CN4CCC[C@@H](Nc5ccc6c(c5)C(=O)N(C5CCC(=O)NC5=O)C6=O)C4)CC3)c2)cnc1N)c1c(Cl)ccc(F)c1Cl] | 5-(((R)-1-((4-(4-(6-amino-5-((R)-1-(2,6- dichloro-3-fluorophenyl)ethoxy)pyridin-3-yl)- 1H-pyrazol-1-yl)cyclohexyl)methyl)piperidin- 3-yl)amino)-2-(2,6-dioxopiperidin-3- yl)isoindoline-1,3-dione |
| | 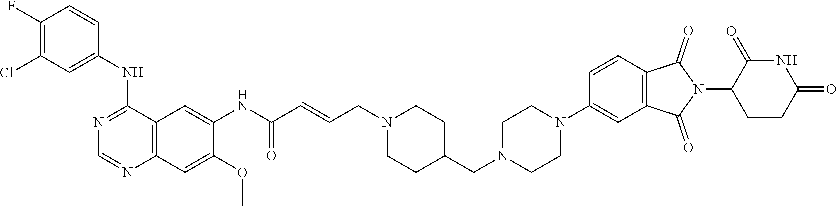 [SMILES: COc1cc2ncnc(Nc3ccc(F)c(Cl)c3)c2cc1NC(=O)/C=C/CN1CCC(CN2CCN(c3ccc4c(c3)C(=O)N(C3CCC(=O)NC3=O)C4=O)CC2)CC1] | (E)-N-(4-((3-chloro-4-fluorophenyl)amino)-7- methoxyquinazolin-6-yl)-4-(4-((4-(2-(2,6- dioxopiperidin-3-yl)-1,3-dioxoisoindolin-5- yl)piperazin-1-yl)methyl)piperidin-1-yl)but-2- enamide |
| | 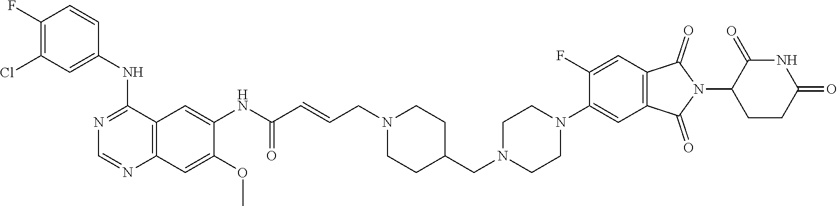 [SMILES: COc1cc2ncnc(Nc3ccc(F)c(Cl)c3)c2cc1NC(=O)/C=C/CN1CCC(CN2CCN(c3cc4c(cc3F)C(=O)N(C3CCC(=O)NC3=O)C4=O)CC2)CC1] | (E)-N-(4-((3-chloro-4-fluorophenyl)amino)-7- methoxyquinazolin-6-yl)-4-(4-((4-(2-(2,6- dioxopiperidin-3-yl)-6-fluoro-1,3- dioxoisoindolin-5-yl)piperazin-1- yl)methyl)piperidin-1-yl)but-2-enamide |
| | 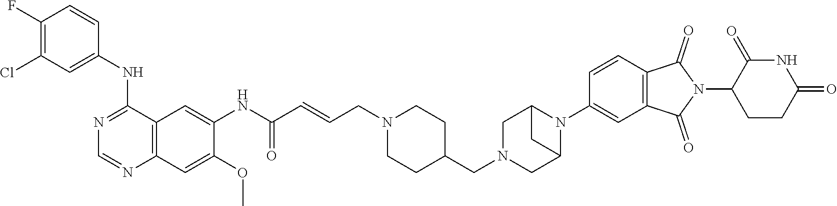 [SMILES: COc1cc2ncnc(Nc3ccc(F)c(Cl)c3)c2cc1NC(=O)/C=C/CN1CCC(CN2CC3CC(C2)N3c2ccc3c(c2)C(=O)N(C2CCC(=O)NC2=O)C3=O)CC1] | (E)-N-(4-((3-chloro-4-fluorophenyl)amino)-7- methoxyquinazolin-6-yl)-4-(4-((6-(2-(2,6- dioxopiperidin-3-yl)-1,3-dioxoisoindolin-5-yl)- 3,6-diazabicyclo[3.1.1]heptan-3- yl)methyl)piperidin-1-yl)but-2-enamide |
| | 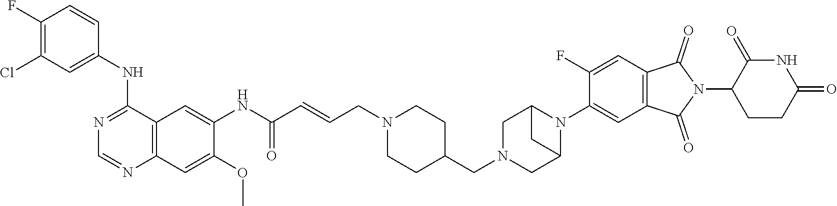 [SMILES: COc1cc2ncnc(Nc3ccc(F)c(Cl)c3)c2cc1NC(=O)/C=C/CN1CCC(CN2CC3CC(C2)N3c2cc3c(cc2F)C(=O)N(C2CCC(=O)NC2=O)C3=O)CC1] | (E)-N-(4-((3-chloro-4-fluorophenyl)amino)-7- methoxyquinazolin-6-yl)-4-(4-((6-(2-(2,6- dioxopiperidin-3-yl)-6-fluoro-1,3- dioxoisoindolin-5-yl)-3,6- diazabicyclo[3.1.1]heptan-3- yl)methyl)piperidin-1-yl)but-2-enamide |
| | 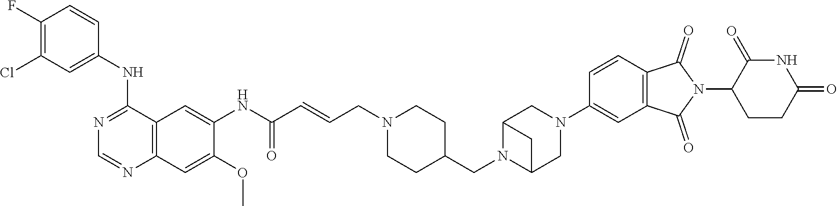 [SMILES: COc1cc2ncnc(Nc3ccc(F)c(Cl)c3)c2cc1NC(=O)/C=C/CN1CCC(CN2C3CC2CN(c2ccc4c(c2)C(=O)N(C2CCC(=O)NC2=O)C4=O)C3)CC1] | (E)-N-(4-((3-chloro-4-fluorophenyl)amino)-7- methoxyquinazolin-6-yl)-4-(4-((3-(2-(2,6- dioxopiperidin-3-yl)-1,3-dioxoisoindolin-5-yl)- 3,6-diazabicyclo[3.1.1]heptan-6- yl)methyl)piperidin-1-yl)but-2-enamide |
| | 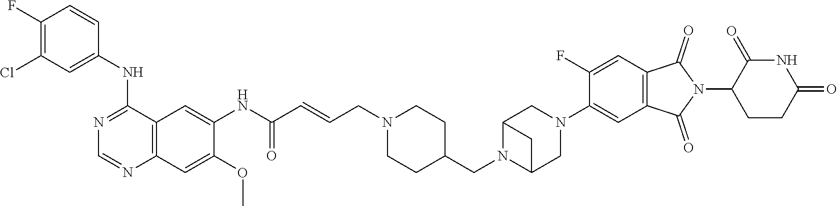 [SMILES: COc1cc2ncnc(Nc3ccc(F)c(Cl)c3)c2cc1NC(=O)/C=C/CN1CCC(CN2C3CC2CN(c2cc4c(cc2F)C(=O)N(C2CCC(=O)NC2=O)C4=O)C3)CC1] | (E)-N-(4-((3-chloro-4-fluorophenyl)amino)-7- methoxyquinazolin-6-yl)-4-(4-((3-(2-(2,6- dioxopiperidin-3-yl)-6-fluoro-1,3- dioxoisoindolin-5-yl)-3,6- diazabicyclo[3.1.1]heptan-6- yl)methyl)piperidin-1-yl)but-2-enamide |
| | 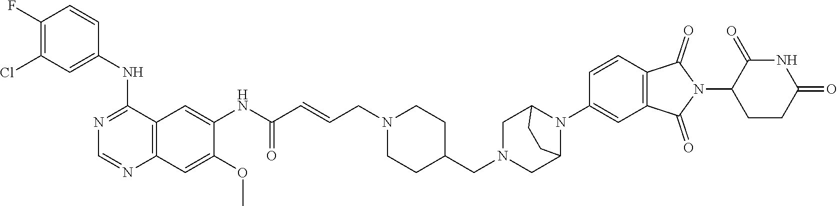 [SMILES: COc1cc2ncnc(Nc3ccc(F)c(Cl)c3)c2cc1NC(=O)/C=C/CN1CCC(CN2CC3CCC(C2)N3c2ccc3c(c2)C(=O)N(C2CCC(=O)NC2=O)C3=O)CC1] | (E)-N-(4-((3-chloro-4-fluorophenyl)amino)-7- methoxyquinazolin-6-yl)-4-(4-((8-(2-(2,6- dioxopiperidin-3-yl)-1,3-dioxoisoindolin-5-yl)- 3,8-diazabicyclo[3.2.1]octan-3- yl)methyl)piperidin-1-yl)but-2-enamide |
| | 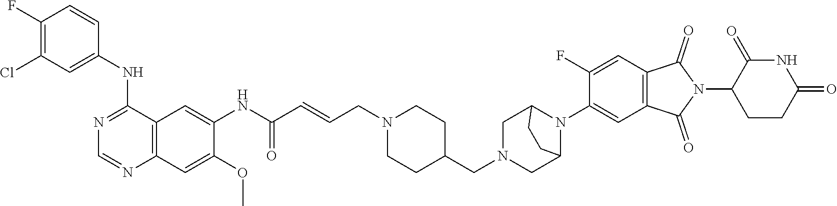 [SMILES: COc1cc2ncnc(Nc3ccc(F)c(Cl)c3)c2cc1NC(=O)/C=C/CN1CCC(CN2CC3CCC(C2)N3c2cc3c(cc2F)C(=O)N(C2CCC(=O)NC2=O)C3=O)CC1] | (E)-N-(4-((3-chloro-4-fluorophenyl)amino)-7- methoxyquinazolin-6-yl)-4-(4-((8-(2-(2,6- dioxopiperidin-3-yl)-6-fluoro-1,3- dioxoisoindolin-5-yl)-3,8- diazabicyclo[3.2.1]octan-3-yl)methyl)piperidin- 1-yl)but-2-enamide |
| | 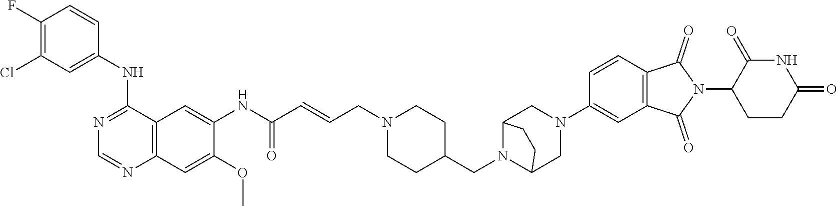 [SMILES: COc1cc2ncnc(Nc3ccc(F)c(Cl)c3)c2cc1NC(=O)/C=C/CN1CCC(CN2C3CCC2CN(c2ccc4c(c2)C(=O)N(C2CCC(=O)NC2=O)C4=O)C3)CC1] | (E)-N-(4-((3-chloro-4-fluorophenyl)amino)-7- methoxyquinazolin-6-yl)-4-(4-((3-(2-(2,6- dioxopiperidin-3-yl)-1,3-dioxoisoindolin-5-yl)- 3,8-diazabicyclo[3.2.1]octan-8- yl)methyl)piperidin-1-yl)but-2-enamide |
| | 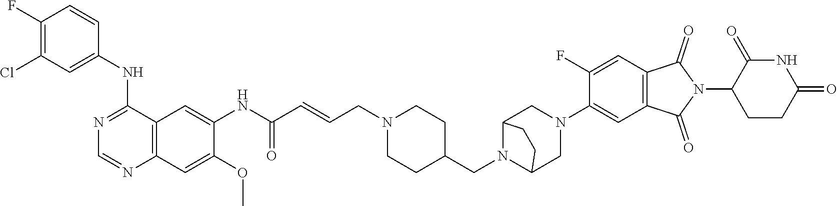 [SMILES: COc1cc2ncnc(Nc3ccc(F)c(Cl)c3)c2cc1NC(=O)/C=C/CN1CCC(CN2C3CCC2CN(c2cc4c(cc2F)C(=O)N(C2CCC(=O)NC2=O)C4=O)C3)CC1] | (E)-N-(4-((3-chloro-4-fluorophenyl)amino)-7- methoxyquinazolin-6-yl)-4-(4-((3-(2-(2,6- dioxopiperidin-3-yl)-6-fluoro-1,3- dioxoisoindolin-5-yl)-3,8- diazabicyclo[3.2.1]octan-8-yl)methyl)piperidin- 1-yl)but-2-enamide |
| | 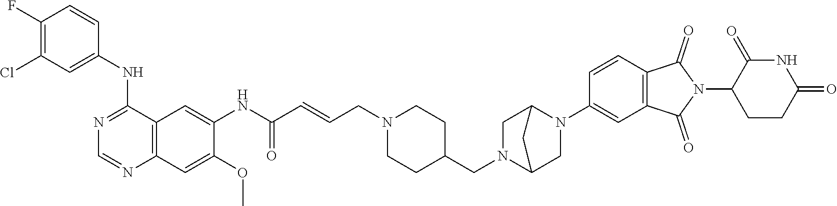 [SMILES: COc1cc2ncnc(Nc3ccc(F)c(Cl)c3)c2cc1NC(=O)/C=C/CN1CCC(CN2CC3CC2CN3c2ccc3c(c2)C(=O)N(C2CCC(=O)NC2=O)C3=O)CC1] | (E)-N-(4-((3-chloro-4-fluorophenyl)amino)-7- methoxyquinazolin-6-yl)-4-(4-((5-(2-(2,6- dioxopiperidin-3-yl)-1,3-dioxoisoindolin-5-yl)- 2,5-diazabicyclo[2.2.1]heptan-2- yl)methyl)piperidin-1-yl)but-2-enamide |
| | 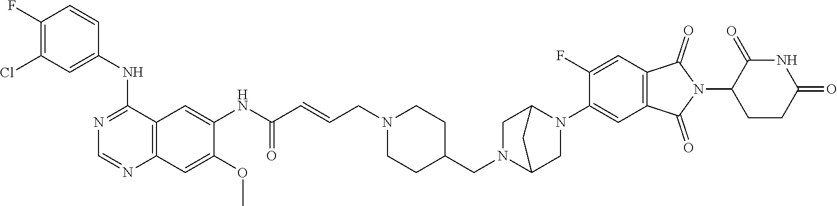 [SMILES: COc1cc2ncnc(Nc3ccc(F)c(Cl)c3)c2cc1NC(=O)/C=C/CN1CCC(CN2CC3CC2CN3c2cc3c(cc2F)C(=O)N(C2CCC(=O)NC2=O)C3=O)CC1] | (E)-N-(4-((3-chloro-4-fluorophenyl)amino)-7- methoxyquinazolin-6-yl)-4-(4-((5-(2-(2,6- dioxopiperidin-3-yl)-6-fluoro-1,3- dioxoisoindolin-5-yl)-2,5- diazabicyclo[2.2.1]heptan-2- yl)methyl)piperidin-1-yl)but-2-enamide |
| | 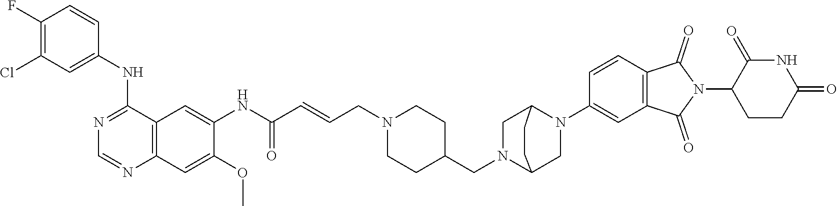 [SMILES: COc1cc2ncnc(Nc3ccc(F)c(Cl)c3)c2cc1NC(=O)/C=C/CN1CCC(CN2CC3CCC2CN3c2ccc3c(c2)C(=O)N(C2CCC(=O)NC2=O)C3=O)CC1] | (E)-N-(4-((3-chloro-4-fluorophenyl)amino)-7- methoxyquinazolin-6-yl)-4-(4-((5-(2-(2,6- dioxopiperidin-3-yl)-1,3-dioxoisoindolin-5-yl)- 2,5-diazabicyclo[2.2.2]octan-2- yl)methyl)piperidin-1-yl)but-2-enamide |
| | 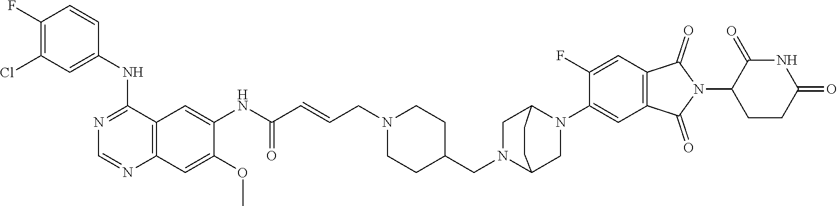 [SMILES: COc1cc2ncnc(Nc3ccc(F)c(Cl)c3)c2cc1NC(=O)/C=C/CN1CCC(CN2CC3CCC2CN3c2cc3c(cc2F)C(=O)N(C2CCC(=O)NC2=O)C3=O)CC1] | (E)-N-(4-((3-chloro-4-fluorophenyl)amino)-7- methoxyquinazolin-6-yl)-4-(4-((5-(2-(2,6- dioxopiperidin-3-yl)-6-fluoro-1,3- dioxoisoindolin-5-yl)-2,5- diazabicyclo[2.2.2]octan-2-yl)methyl)piperidin- 1-yl)but-2-enamide |
| | 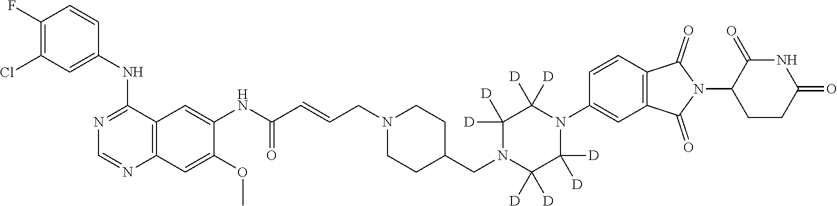 [SMILES: [2H]C1([2H])N(CC2CCN(C/C=C/C(=O)Nc3cc4c(Nc5ccc(F)c(Cl)c5)ncnc4cc3OC)CC2)C([2H])([2H])C([2H])([2H])N(c2ccc3c(c2)C(=O)N(C2CCC(=O)NC2=O)C3=O)C1([2H])[2H]] | (E)-N-(4-((3-chloro-4-fluorophenyl)amino)-7- methoxyquinazolin-6-yl)-4-(4-((4-(2-(2,6- dioxopiperidin-3-yl)-1,3-dioxoisoindolin-5- yl)piperazin-1-yl-2,2,3,3,5,5,6,6- d₈)methyl)piperidin-1-yl)but-2-enamide |
| | 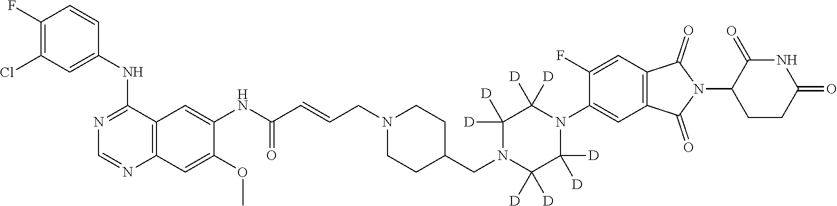 [SMILES: [2H]C1([2H])N(CC2CCN(C/C=C/C(=O)Nc3cc4c(Nc5ccc(F)c(Cl)c5)ncnc4cc3OC)CC2)C([2H])([2H])C([2H])([2H])N(c2cc3c(cc2F)C(=O)N(C2CCC(=O)NC2=O)C3=O)C1([2H])[2H]] | (E)-N-(4-((3-chloro-4-fluorophenyl)amino)-7- methoxyquinazolin-6-yl)-4-(4-((4-(2-(2,6- dioxopiperidin-3-yl)-6-fluoro-1,3- dioxoisoindolin-5-yl)piperazin-1-yl- 2,2,3,3,5,5,6,6-d₈)methyl)piperidin-1-yl)but-2- enamide |
| | 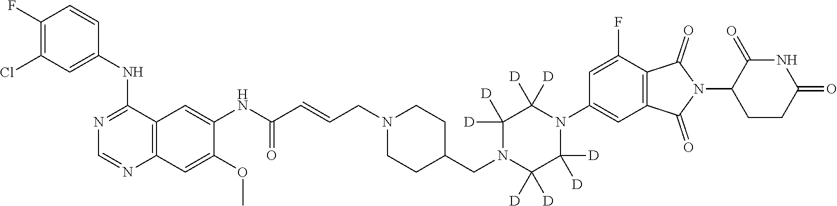 [SMILES: [2H]C1([2H])N(CC2CCN(C/C=C/C(=O)Nc3cc4c(Nc5ccc(F)c(Cl)c5)ncnc4cc3OC)CC2)C([2H])([2H])C([2H])([2H])N(c2cc(F)c3c(c2)C(=O)N(C2CCC(=O)NC2=O)C3=O)C1([2H])[2H]] | (E)-N-(4-((3-chloro-4-fluorophenyl)amino)-7- methoxyquinazolin-6-yl)-4-(4-((4-(2-(2,6- dioxopiperidin-3-yl)-7-fluoro-1,3- dioxoisoindolin-5-yl)piperazin-1-yl- 2,2,3,3,5,5,6,6-d₈)methyl)piperidin-1-yl)but-2- enamide |
| | 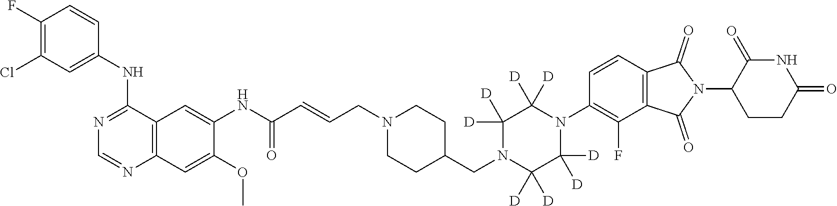 [SMILES: [2H]C1([2H])N(CC2CCN(C/C=C/C(=O)Nc3cc4c(Nc5ccc(F)c(Cl)c5)ncnc4cc3OC)CC2)C([2H])([2H])C([2H])([2H])N(c2ccc3c(c2F)C(=O)N(C2CCC(=O)NC2=O)C3=O)C1([2H])[2H]] | (E)-N-(4-((3-chloro-4-fluorophenyl)amino)-7- methoxyquinazolin-6-yl)-4-(4-((4-(2-(2,6- dioxopiperidin-3-yl)-4-fluoro-1,3- dioxoisoindolin-5-yl)piperazin-1-yl- 2,2,3,3,5,5,6,6-d₈)methyl)piperidin-1-yl)but-2- enamide |
| | 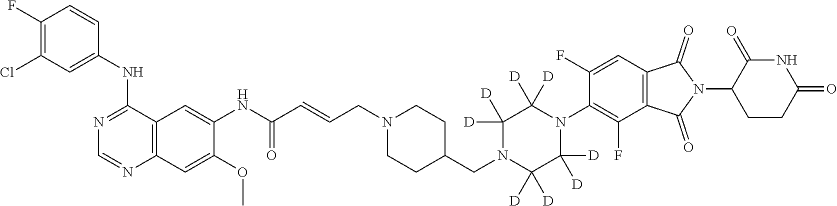 [SMILES: [2H]C1([2H])N(CC2CCN(C/C=C/C(=O)Nc3cc4c(Nc5ccc(F)c(Cl)c5)ncnc4cc3OC)CC2)C([2H])([2H])C([2H])([2H])N(c2c(F)cc3c(c2F)C(=O)N(C2CCC(=O)NC2=O)C3=O)C1([2H])[2H]] | (E)-N-(4-((3-chloro-4-fluorophenyl)amino)-7- methoxyquinazolin-6-yl)-4-(4-((4-(2-(2,6- dioxopiperidin-3-yl)-4,6-difluoro-1,3- dioxoisoindolin-5-yl)piperazin-1-yl- 2,2,3,3,5,5,6,6-d₈)methyl)piperidin-1-yl)but-2- enamide |
| | 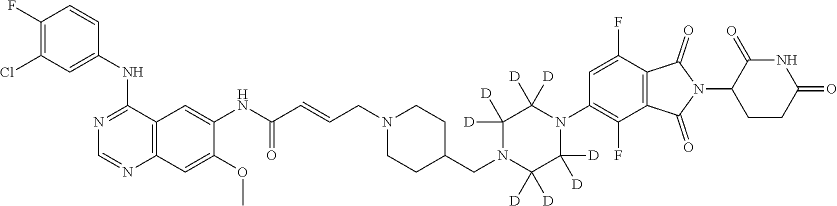 [SMILES: [2H]C1([2H])N(CC2CCN(C/C=C/C(=O)Nc3cc4c(Nc5ccc(F)c(Cl)c5)ncnc4cc3OC)CC2)C([2H])([2H])C([2H])([2H])N(c2cc(F)c3c(c2F)C(=O)N(C2CCC(=O)NC2=O)C3=O)C1([2H])[2H]] | (E)-N-(4-((3-chloro-4-fluorophenyl)amino)-7- methoxyquinazolin-6-yl)-4-(4-((4-(2-(2,6- dioxopiperidin-3-yl)-4,7-difluoro-1,3- dioxoisoindolin-5-yl)piperazin-1-yl- 2,2,3,3,5,5,6,6-d₈)methyl)piperidin-1-yl)but-2- enamide |
| | 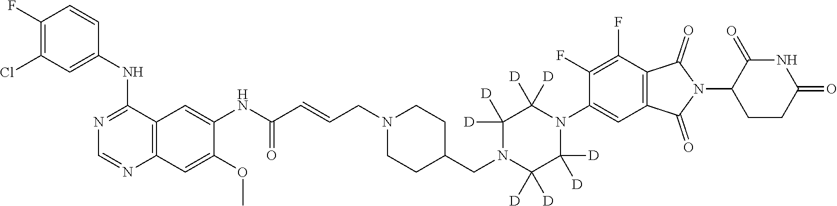 [SMILES: [2H]C1([2H])N(CC2CCN(C/C=C/C(=O)Nc3cc4c(Nc5ccc(F)c(Cl)c5)ncnc4cc3OC)CC2)C([2H])([2H])C([2H])([2H])N(c2cc3c(c(F)c2F)C(=O)N(C2CCC(=O)NC2=O)C3=O)C1([2H])[2H]] | (E)-N-(4-((3-chloro-4-fluorophenyl)amino)-7- methoxyquinazolin-6-yl)-4-(4-((4-(2-(2,6- dioxopiperidin-3-yl)-6,7-difluoro-1,3- dioxoisoindolin-5-yl)piperazin-1-yl- 2,2,3,3,5,5,6,6-d₈)methyl)piperidin-1-yl)but-2- enamide |
| | 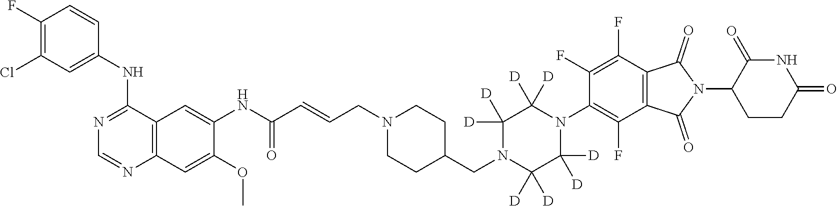 [SMILES: [2H]C1([2H])N(CC2CCN(C/C=C/C(=O)Nc3cc4c(Nc5ccc(F)c(Cl)c5)ncnc4cc3OC)CC2)C([2H])([2H])C([2H])([2H])N(c2c(F)c(F)c3c(c2F)C(=O)N(C2CCC(=O)NC2=O)C3=O)C1([2H])[2H]] | (E)-N-(4-((3-chloro-4-fluorophenyl)amino)-7- methoxyquinazolin-6-yl)-4-(4-((4-(2-(2,6- dioxopiperidin-3-yl)-4,6,7-trifluoro-1,3- dioxoisoindolin-5-yl)piperazin-1-yl- 2,2,3,3,5,5,6,6-d₈)methyl)piperidin-1-yl)but-2- enamide |
| | 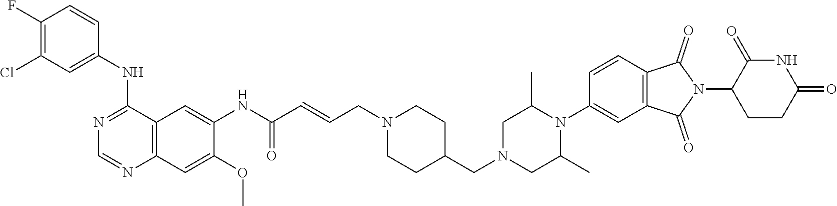 [SMILES: COc1cc2ncnc(Nc3ccc(F)c(Cl)c3)c2cc1NC(=O)/C=C/CN1CCC(CN2CC(C)N(c3ccc4c(c3)C(=O)N(C3CCC(=O)NC3=O)C4=O)C(C)C2)CC1] | (E)-N-(4-((3-chloro-4-fluorophenyl)amino)-7- methoxyquinazolin-6-yl)-4-(4-((4-(2-(2,6- dioxopiperidin-3-yl)-1,3-dioxoisoindolin-5-yl)- 3,5-dimethylpiperazin-1-yl)methyl)piperidin-1- yl)but-2-enamide |
| | 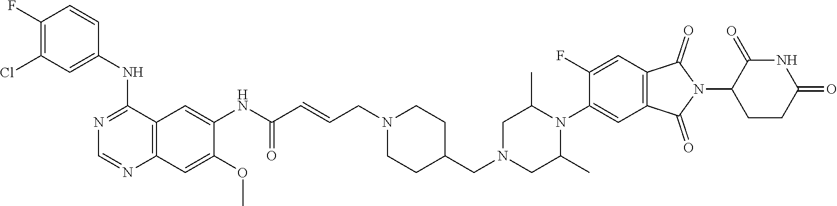 [SMILES: COc1cc2ncnc(Nc3ccc(F)c(Cl)c3)c2cc1NC(=O)/C=C/CN1CCC(CN2CC(C)N(c3cc4c(cc3F)C(=O)N(C3CCC(=O)NC3=O)C4=O)C(C)C2)CC1] | (E)-N-(4-((3-chloro-4-fluorophenyl)amino)-7- methoxyquinazolin-6-yl)-4-(4-((4-(2-(2,6- dioxopiperidin-3-yl)-6-fluoro-1,3- dioxoisoindolin-5-yl)-3,5-dimethylpiperazin-1- yl)methyl)piperidin-1-yl)but-2-enamide |
| | 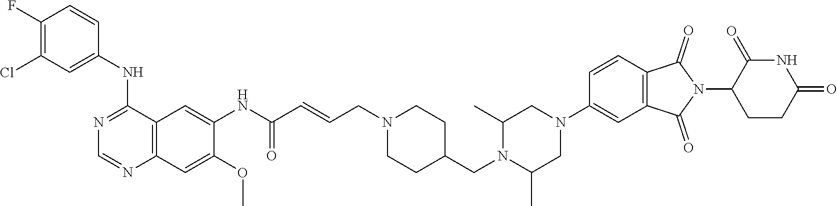 [SMILES: COc1cc2ncnc(Nc3ccc(F)c(Cl)c3)c2cc1NC(=O)/C=C/CN1CCC(CN2C(C)CN(c3ccc4c(c3)C(=O)N(C3CCC(=O)NC3=O)C4=O)CC2C)CC1] | (E)-N-(4-((3-chloro-4-fluorophenyl)amino)-7- methoxyquinazolin-6-yl)-4-(4-((4-(2-(2,6- dioxopiperidin-3-yl)-1,3-dioxoisoindolin-5-yl)- 2,6-dimethylpiperazin-1-yl)methyl)piperidin-1- yl)but-2-enamide |
| | 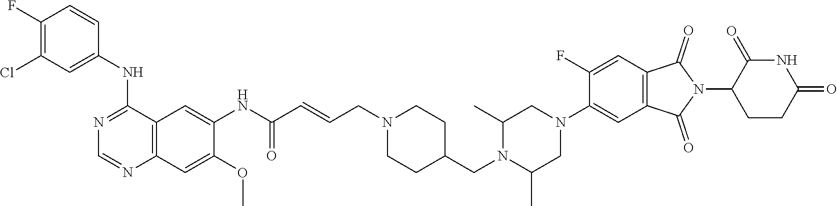 [SMILES: COc1cc2ncnc(Nc3ccc(F)c(Cl)c3)c2cc1NC(=O)/C=C/CN1CCC(CN2C(C)CN(c3cc4c(cc3F)C(=O)N(C3CCC(=O)NC3=O)C4=O)CC2C)CC1] | (E)-N-(4-((3-chloro-4-fluorophenyl)amino)-7- methoxyquinazolin-6-yl)-4-(4-((4-(2-(2,6- dioxopiperidin-3-yl)-6-fluoro-1,3- dioxoisoindolin-5-yl)-2,6-dimethylpiperazin-1- yl)methyl)piperidin-1-yl)but-2-enamide |
| | 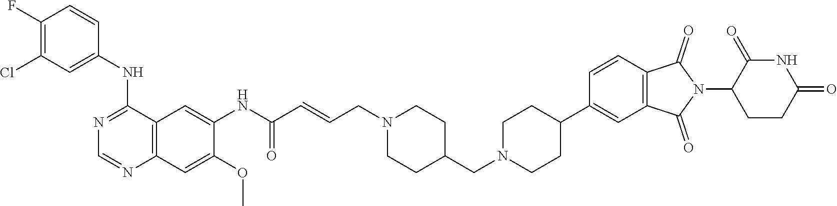 [SMILES: COc1cc2ncnc(Nc3ccc(F)c(Cl)c3)c2cc1NC(=O)/C=C/CN1CCC(CN2CCC(c3ccc4c(c3)C(=O)N(C3CCC(=O)NC3=O)C4=O)CC2)CC1] | (E)-N-(4-((3-chloro-4-fluorophenyl)amino)-7- methoxyquinazolin-6-yl)-4-(4-((4-(2-(2,6- dioxopiperidin-3-yl)-1,3-dioxoisoindolin-5- yl)piperidin-1-yl)methyl)piperidin-1-yl)but-2- enamide |
| | 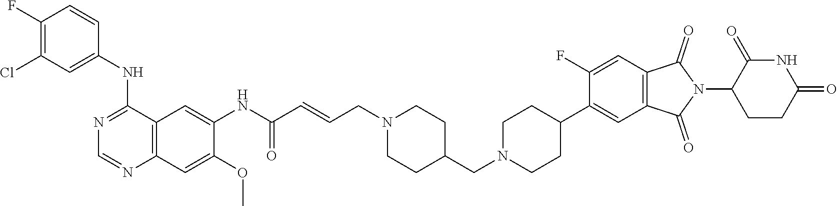 [SMILES: COc1cc2ncnc(Nc3ccc(F)c(Cl)c3)c2cc1NC(=O)/C=C/CN1CCC(CN2CCC(c3cc4c(cc3F)C(=O)N(C3CCC(=O)NC3=O)C4=O)CC2)CC1] | (E)-N-(4-((3-chloro-4-fluorophenyl)amino)-7- methoxyquinazolin-6-yl)-4-(4-((4-(2-(2,6- dioxopiperidin-3-yl)-6-fluoro-1,3- dioxoisoindolin-5-yl)piperidin-1- yl)methyl)piperidin-1-yl)but-2-enamide |
| | 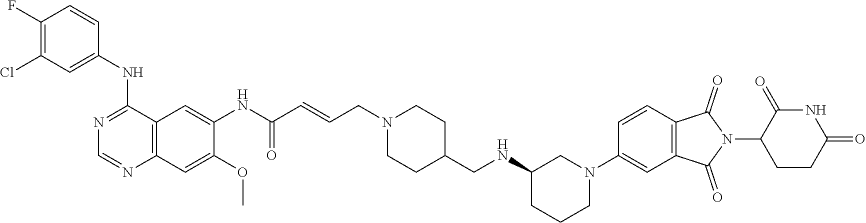 [SMILES: COc1cc2ncnc(Nc3ccc(F)c(Cl)c3)c2cc1NC(=O)/C=C/CN1CCC(CN[C@@H]2CCCN(c3ccc4c(c3)C(=O)N(C3CCC(=O)NC3=O)C4=O)C2)CC1] | (E)-N-(4-((3-chloro-4-fluorophenyl)amino)-7- methoxyquinazolin-6-yl)-4-(4-((((3R)-1-(2- (2,6-dioxopiperidin-3-yl)-1,3-dioxoisoindolin- 5-yl)piperidin-3-yl)amino)methyl)piperidin-1- yl)but-2-enamide |
| | 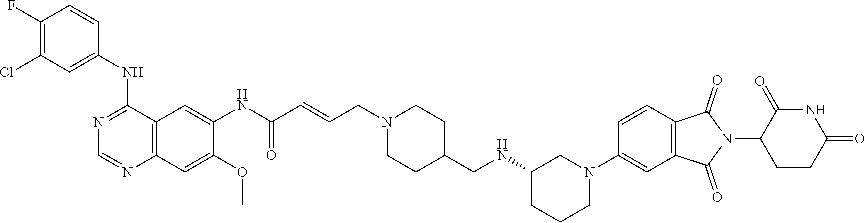 [SMILES: COc1cc2ncnc(Nc3ccc(F)c(Cl)c3)c2cc1NC(=O)/C=C/CN1CCC(CN[C@H]2CCCN(c3ccc4c(c3)C(=O)N(C3CCC(=O)NC3=O)C4=O)C2)CC1] | (E)-N-(4-((3-chloro-4-fluorophenyl)amino)-7- methoxyquinazolin-6-yl)-4-(4-((((3S)-1-(2- (2,6-dioxopiperidin-3-yl)-1,3-dioxoisoindolin- 5-yl)piperidin-3-yl)amino)methyl)piperidin-1- yl)but-2-enamide |
| | 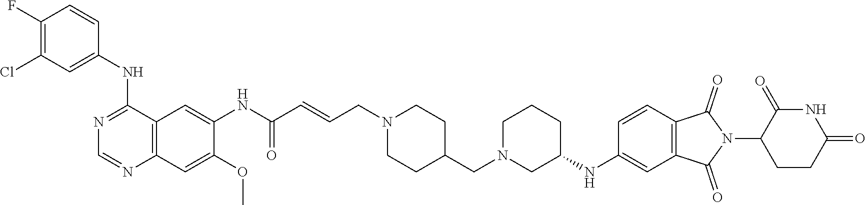 [SMILES: COc1cc2ncnc(Nc3ccc(F)c(Cl)c3)c2cc1NC(=O)/C=C/CN1CCC(CN2CCC[C@H](Nc3ccc4c(c3)C(=O)N(C3CCC(=O)NC3=O)C4=O)C2)CC1] | (E)-N-(4-((3-chloro-4-fluorophenyl)amino)-7- methoxyquinazolin-6-yl)-4-(4-(((3S)-3-((2- (2,6-dioxopiperidin-3-yl)-1,3-dioxoisoindolin- 5-yl)amino)piperidin-1-yl)methyl)piperidin-1- yl)but-2-enamide |
| | 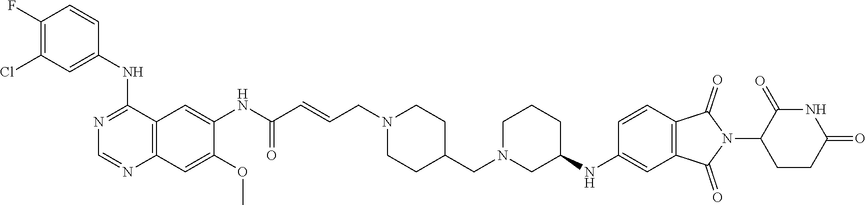 [SMILES: COc1cc2ncnc(Nc3ccc(F)c(Cl)c3)c2cc1NC(=O)/C=C/CN1CCC(CN2CCC[C@@H](Nc3ccc4c(c3)C(=O)N(C3CCC(=O)NC3=O)C4=O)C2)CC1] | (E)-N-(4-((3-chloro-4-fluorophenyl)amino)-7- methoxyquinazolin-6-yl)-4-(4-(((3R)-3-((2- (2,6-dioxopiperidin-3-yl)-1,3-dioxoisoindolin- 5-yl)amino)piperidin-1-yl)methyl)piperidin-1- yl)but-2-enamide |
| | 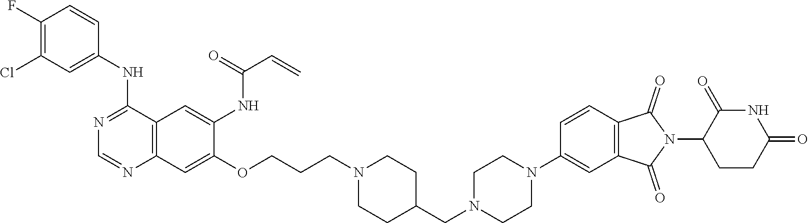 [SMILES: C=CC(=O)Nc1cc2c(Nc3ccc(F)c(Cl)c3)ncnc2cc1OCCCN1CCC(CN2CCN(c3ccc4c(c3)C(=O)N(C3CCC(=O)NC3=O)C4=O)CC2)CC1] | N-(4-((3-chloro-4-fluorophenyl)amino)-7-(3- (4-((4-(2-(2,6-dioxopiperidin-3-yl)-1,3- dioxoisoindolin-5-yl)piperazin-1- yl)methyl)piperidin-1-yl)propoxy)quinazolin-6- yl)acrylamide |
| | 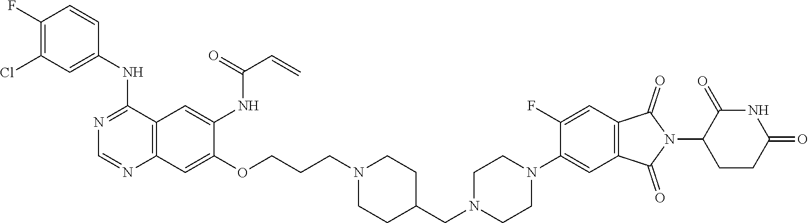 [SMILES: C=CC(=O)Nc1cc2c(Nc3ccc(F)c(Cl)c3)ncnc2cc1OCCCN1CCC(CN2CCN(c3cc4c(cc3F)C(=O)N(C3CCC(=O)NC3=O)C4=O)CC2)CC1] | N-(4-((3-chloro-4-fluorophenyl)amino)-7-(3- (4-((4-(2-(2,6-dioxopiperidin-3-yl)-6-fluoro- 1,3-dioxoisoindolin-5-yl)piperazin-1- yl)methyl)piperidin-1-yl)propoxy)quinazolin-6- yl)acrylamide |
| | 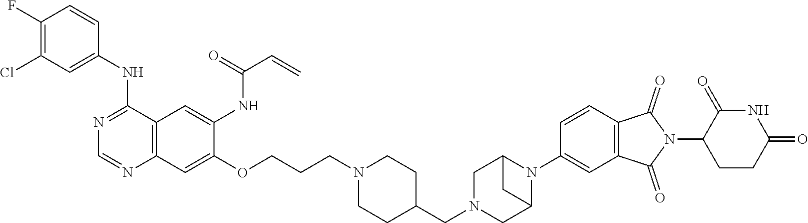 [SMILES: C=CC(=O)Nc1cc2c(Nc3ccc(F)c(Cl)c3)ncnc2cc1OCCCN1CCC(CN2CC3CC(C2)N3c2ccc3c(c2)C(=O)N(C2CCC(=O)NC2=O)C3=O)CC1] | N-(4-((3-chloro-4-fluorophenyl)amino)-7-(3- (4-((6-(2-(2,6-dioxopiperidin-3-yl)-1,3- dioxoisoindolin-5-yl)-3,6- diazabicyclo[3.1.1]heptan-3- yl)methyl)piperidin-1-yl)propoxy)quinazolin-6- yl)acrylamide |
| | 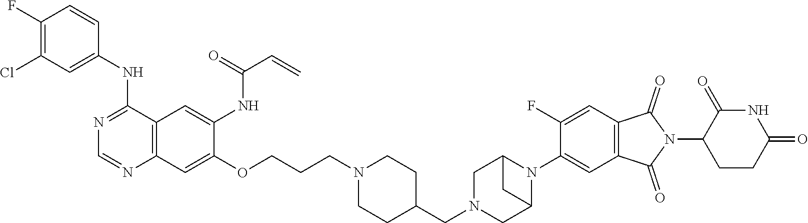 [SMILES: C=CC(=O)Nc1cc2c(Nc3ccc(F)c(Cl)c3)ncnc2cc1OCCCN1CCC(CN2CC3CC(C2)N3c2cc3c(cc2F)C(=O)N(C2CCC(=O)NC2=O)C3=O)CC1] | N-(4-((3-chloro-4-fluorophenyl)amino)-7-(3- (4-((6-(2-(2,6-dioxopiperidin-3-yl)-6-fluoro- 1,3-dioxoisoindolin-5-yl)-3,6- diazabicyclo[3.1.1]heptan-3- yl)methyl)piperidin-1-yl)propoxy)quinazolin-6- yl)acrylamide |
| | 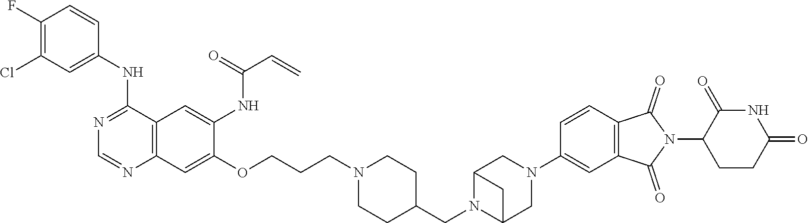 [SMILES: C=CC(=O)Nc1cc2c(Nc3ccc(F)c(Cl)c3)ncnc2cc1OCCCN1CCC(CN2C3CC2CN(c2ccc4c(c2)C(=O)N(C2CCC(=O)NC2=O)C4=O)C3)CC1] | N-(4-((3-chloro-4-fluorophenyl)amino)-7-(3- (4-((3-(2-(2,6-dioxopiperidin-3-yl)-1,3- dioxoisoindolin-5-yl)-3,6- diazabicyclo[3.1.1]heptan-6- yl)methyl)piperidin-1-yl)propoxy)quinazolin-6- yl)acrylamide |
| | 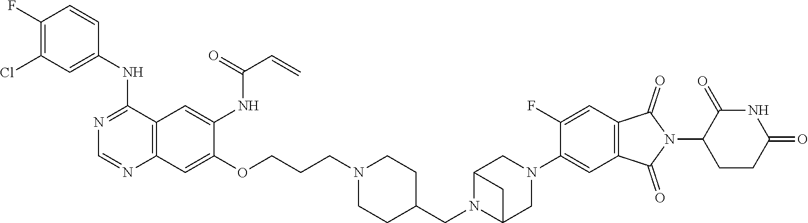 [SMILES: C=CC(=O)Nc1cc2c(Nc3ccc(F)c(Cl)c3)ncnc2cc1OCCCN1CCC(CN2C3CC2CN(c2cc4c(cc2F)C(=O)N(C2CCC(=O)NC2=O)C4=O)C3)CC1] | N-(4-((3-chloro-4-fluorophenyl)amino)-7-(3- (4-((3-(2-(2,6-dioxopiperidin-3-yl)-6-fluoro- 1,3-dioxoisoindolin-5-yl)-3,6- diazabicyclo[3.1.1]heptan-6- yl)methyl)piperidin-1-yl)propoxy)quinazolin-6- yl)acrylamide |
| | 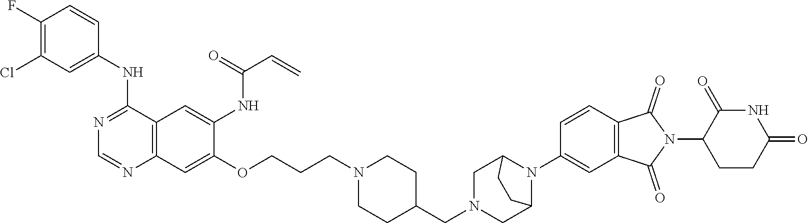 [SMILES: C=CC(=O)Nc1cc2c(Nc3ccc(F)c(Cl)c3)ncnc2cc1OCCCN1CCC(CN2CC3CCC(C2)N3c2ccc3c(c2)C(=O)N(C2CCC(=O)NC2=O)C3=O)CC1] | N-(4-((3-chloro-4-fluorophenyl)amino)-7-(3- (4-((8-(2-(2,6-dioxopiperidin-3-yl)-1,3- dioxoisoindolin-5-yl)-3,8- diazabicyclo[3.2.1]octan-3-yl)methyl)piperidin- 1-yl)propoxy)quinazolin-6-yl)acrylamide |
| | 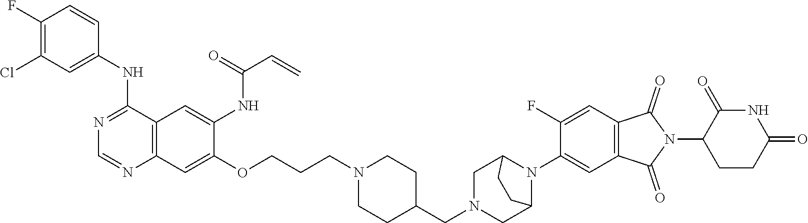 [SMILES: C=CC(=O)Nc1cc2c(Nc3ccc(F)c(Cl)c3)ncnc2cc1OCCCN1CCC(CN2CC3CCC(C2)N3c2cc3c(cc2F)C(=O)N(C2CCC(=O)NC2=O)C3=O)CC1] | N-(4-((3-chloro-4-fluorophenyl)amino)-7-(3- (4-((8-(2-(2,6-dioxopiperidin-3-yl)-6-fluoro- 1,3-dioxoisoindolin-5-yl)-3,8- diazabicyclo[3.2.1]octan-3-yl)methyl)piperidin- 1-yl)propoxy)quinazolin-6-yl)acrylamide |
| | 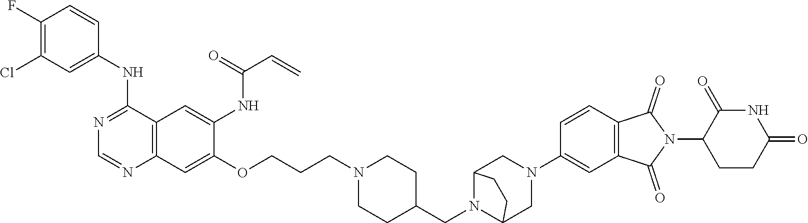 [SMILES: C=CC(=O)Nc1cc2c(Nc3ccc(F)c(Cl)c3)ncnc2cc1OCCCN1CCC(CN2C3CCC2CN(c2ccc4c(c2)C(=O)N(C2CCC(=O)NC2=O)C4=O)C3)CC1] | N-(4-((3-chloro-4-fluorophenyl)amino)-7-(3- (4-((3-(2-(2,6-dioxopiperidin-3-yl)-1,3- dioxoisoindolin-5-yl)-3,8- diazabicyclo[3.2.1]octan-8-yl)methyl)piperidin- 1-yl)propoxy)quinazolin-6-yl)acrylamide |
| | 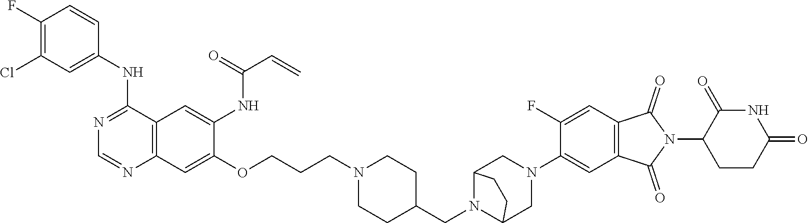 [SMILES: C=CC(=O)Nc1cc2c(Nc3ccc(F)c(Cl)c3)ncnc2cc1OCCCN1CCC(CN2C3CCC2CN(c2cc4c(cc2F)C(=O)N(C2CCC(=O)NC2=O)C4=O)C3)CC1] | N-(4-((3-chloro-4-fluorophenyl)amino)-7-(3- (4-((3-(2-(2,6-dioxopiperidin-3-yl)-6-fluoro- 1,3-dioxoisoindolin-5-yl)-3,8- diazabicyclo[3.2.1]octan-8-yl)methyl)piperidin- 1-yl)propoxy)quinazolin-6-yl)acrylamide |
| | 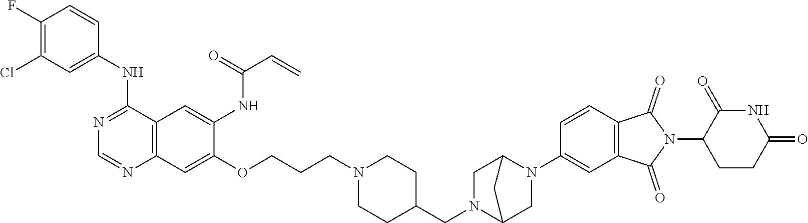 [SMILES: C=CC(=O)Nc1cc2c(Nc3ccc(F)c(Cl)c3)ncnc2cc1OCCCN1CCC(CN2CC3CC2CN3c2ccc3c(c2)C(=O)N(C2CCC(=O)NC2=O)C3=O)CC1] | N-(4-((3-chloro-4-fluorophenyl)amino)-7-(3- (4-((5-(2-(2,6-dioxopiperidin-3-yl)-1,3- dioxoisoindolin-5-yl)-2,5- diazabicyclo[2.2.1]heptan-2- yl)methyl)piperidin-1-yl)propoxy)quinazolin-6- yl)acrylamide |
| | 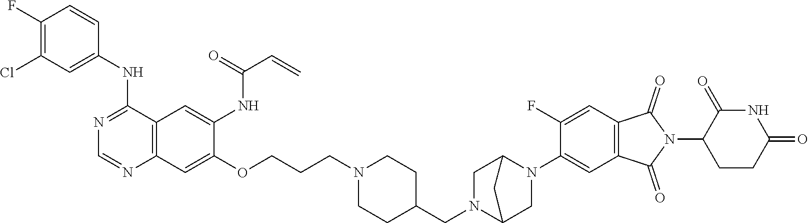 [SMILES: C=CC(=O)Nc1cc2c(Nc3ccc(F)c(Cl)c3)ncnc2cc1OCCCN1CCC(CN2CC3CC2CN3c2cc3c(cc2F)C(=O)N(C2CCC(=O)NC2=O)C3=O)CC1] | N-(4-((3-chloro-4-fluorophenyl)amino)-7-(3- (4-((5-(2-(2,6-dioxopiperidin-3-yl)-6-fluoro- 1,3-dioxoisoindolin-5-yl)-2,5- diazabicyclo[2.2.1]heptan-2- yl)methyl)piperidin-1-yl)propoxy)quinazolin-6- yl)acrylamide |
| | 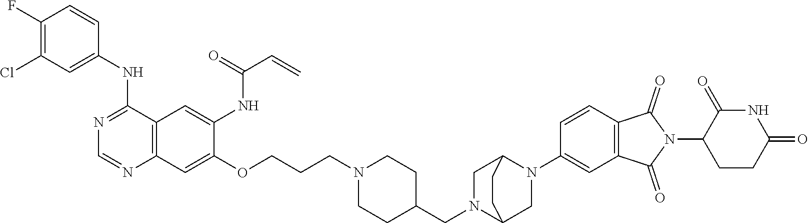 [SMILES: C=CC(=O)Nc1cc2c(Nc3ccc(F)c(Cl)c3)ncnc2cc1OCCCN1CCC(CN2CC3CCC2CN3c2ccc3c(c2)C(=O)N(C2CCC(=O)NC2=O)C3=O)CC1] | N-(4-((3-chloro-4-fluorophenyl)amino)-7-(3- (4-((5-(2-(2,6-dioxopiperidin-3-yl)-1,3- dioxoisoindolin-5-yl)-2,5- diazabicyclo[2.2.2]octan-2-yl)methyl)piperidin- 1-yl)propoxy)quinazolin-6-yl)acrylamide |
| | 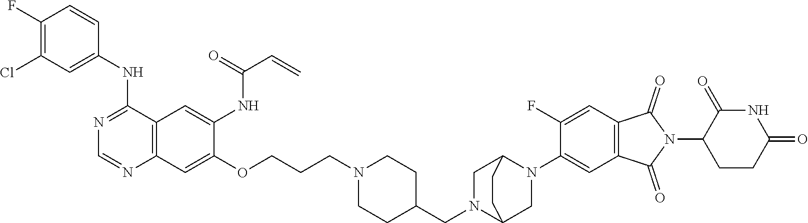 [SMILES: C=CC(=O)Nc1cc2c(Nc3ccc(F)c(Cl)c3)ncnc2cc1OCCCN1CCC(CN2CC3CCC2CN3c2cc3c(cc2F)C(=O)N(C2CCC(=O)NC2=O)C3=O)CC1] | N-(4-((3-chloro-4-fluorophenyl)amino)-7-(3- (4-((5-(2-(2,6-dioxopiperidin-3-yl)-6-fluoro- 1,3-dioxoisoindolin-5-yl)-2,5- diazabicyclo[2.2.2]octan-2-yl)methyl)piperidin- 1-yl)propoxy)quinazolin-6-yl)acrylamide |
| | 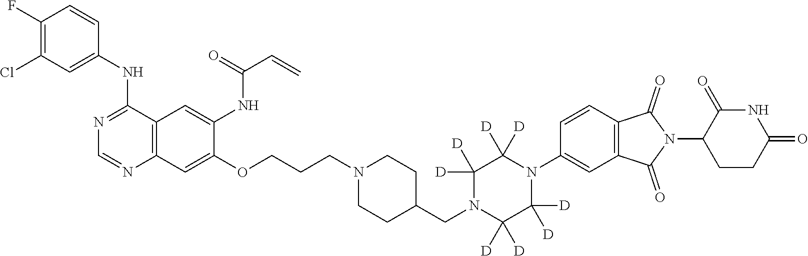 [SMILES: [2H]C1([2H])N(CC2CCN(CCCOc3cc4ncnc(Nc5ccc(F)c(Cl)c5)c4cc3NC(=O)C=C)CC2)C([2H])([2H])C([2H])([2H])N(c2ccc3c(c2)C(=O)N(C2CCC(=O)NC2=O)C3=O)C1([2H])[2H]] | N-(4-((3-chloro-4-fluorophenyl)amino)-7-(3- (4-((4-(2-(2,6-dioxopiperidin-3-yl)-1,3- dioxoisoindolin-5-yl)piperazin-1-yl- 2,2,3,3,5,5,6,6-d₈)methyl)piperidin-1- yl)propoxy)quinazolin-6-yl)acrylamide |
| | 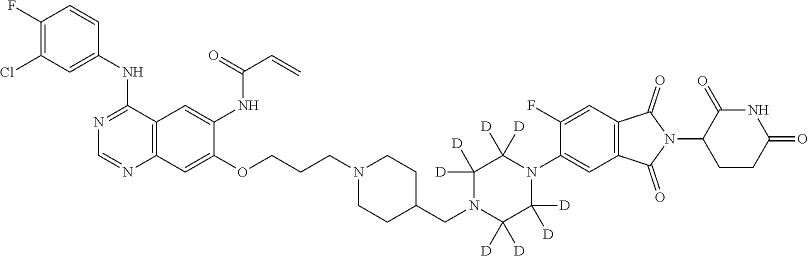 [SMILES: [2H]C1([2H])N(CC2CCN(CCCOc3cc4ncnc(Nc5ccc(F)c(Cl)c5)c4cc3NC(=O)C=C)CC2)C([2H])([2H])C([2H])([2H])N(c2cc3c(cc2F)C(=O)N(C2CCC(=O)NC2=O)C3=O)C1([2H])[2H]] | N-(4-((3-chloro-4-fluorophenyl)amino)-7-(3- (4-((4-(2-(2,6-dioxopiperidin-3-yl)-6-fluoro- 1,3-dioxoisoindolin-5-yl)piperazin-1-yl- 2,2,3,3,5,5,6,6-d₈)methyl)piperidin-1- yl)propoxy)quinazolin-6-yl)acrylamide |
| | 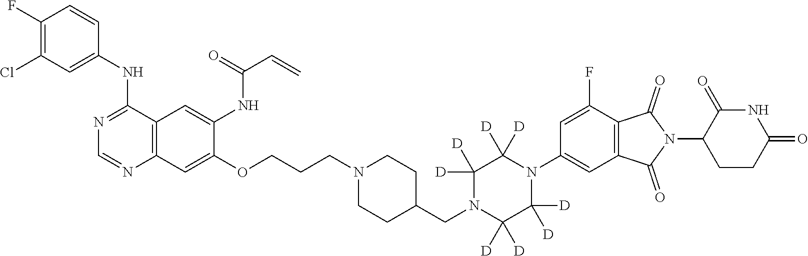 [SMILES: [2H]C1([2H])N(CC2CCN(CCCOc3cc4ncnc(Nc5ccc(F)c(Cl)c5)c4cc3NC(=O)C=C)CC2)C([2H])([2H])C([2H])([2H])N(c2cc(F)c3c(c2)C(=O)N(C2CCC(=O)NC2=O)C3=O)C1([2H])[2H]] | N-(4-((3-chloro-4-fluorophenyl)amino)-7-(3- (4-((4-(2-(2,6-dioxopiperidin-3-yl)-7-fluoro- 1,3-dioxoisoindolin-5-yl)piperazin-1-yl- 2,2,3,3,5,5,6,6-d₈)methyl)piperidin-1- yl)propoxy)quinazolin-6-yl)acrylamide |
| | 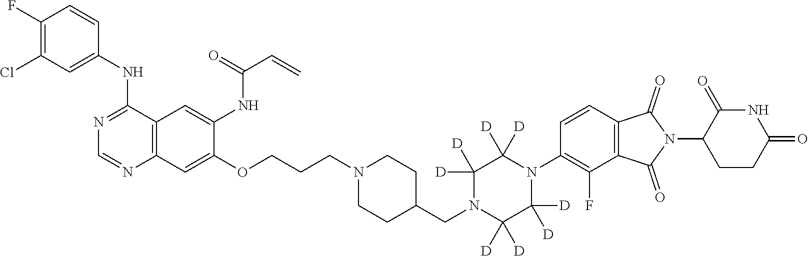 [SMILES: [2H]C1([2H])N(CC2CCN(CCCOc3cc4ncnc(Nc5ccc(F)c(Cl)c5)c4cc3NC(=O)C=C)CC2)C([2H])([2H])C([2H])([2H])N(c2ccc3c(c2F)C(=O)N(C2CCC(=O)NC2=O)C3=O)C1([2H])[2H]] | N-(4-((3-chloro-4-fluorophenyl)amino)-7-(3- (4-((4-(2-(2,6-dioxopiperidin-3-yl)-4-fluoro- 1,3-dioxoisoindolin-5-yl)piperazin-1-yl- 2,2,3,3,5,5,6,6-d₈)methyl)piperidin-1- yl)propoxy)quinazolin-6-yl)acrylamide |
| | 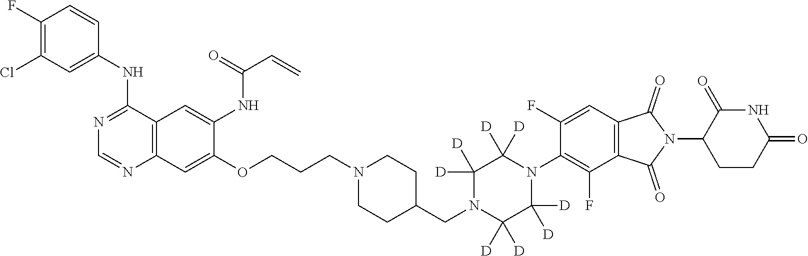 [SMILES: [2H]C1([2H])N(CC2CCN(CCCOc3cc4ncnc(Nc5ccc(F)c(Cl)c5)c4cc3NC(=O)C=C)CC2)C([2H])([2H])C([2H])([2H])N(c2c(F)cc3c(c2F)C(=O)N(C2CCC(=O)NC2=O)C3=O)C1([2H])[2H]] | N-(4-((3-chloro-4-fluorophenyl)amino)-7-(3- (4-((4-(2-(2,6-dioxopiperidin-3-yl)-4,6- difluoro-1,3-dioxoisoindolin-5-yl)piperazin-1- yl-2,2,3,3,5,5,6,6-d₈)methyl)piperidin-1- yl)propoxy)quinazolin-6-yl)acrylamide |
| | 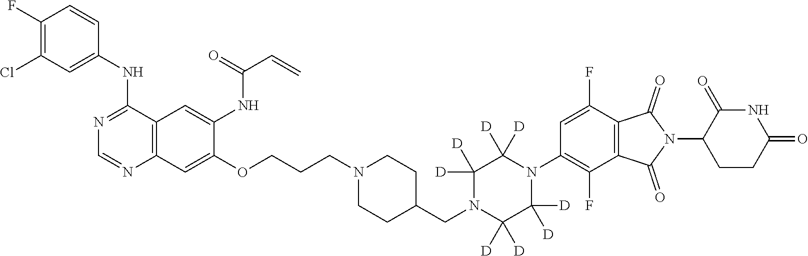 [SMILES: [2H]C1([2H])N(CC2CCN(CCCOc3cc4ncnc(Nc5ccc(F)c(Cl)c5)c4cc3NC(=O)C=C)CC2)C([2H])([2H])C([2H])([2H])N(c2cc(F)c3c(c2F)C(=O)N(C2CCC(=O)NC2=O)C3=O)C1([2H])[2H]] | N-(4-((3-chloro-4-fluorophenyl)amino)-7-(3- (4-((4-(2-(2,6-dioxopiperidin-3-yl)-4,7- difluoro-1,3-dioxoisoindolin-5-yl)piperazin-1- yl-2,2,3,3,5,5,6,6-d₈)methyl)piperidin-1- yl)propoxy)quinazolin-6-yl)acrylamide |
| | 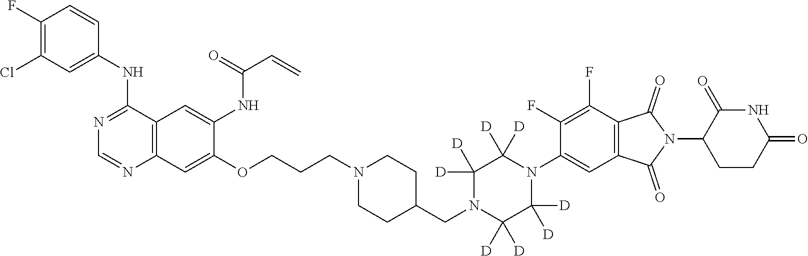 [SMILES: [2H]C1([2H])N(CC2CCN(CCCOc3cc4ncnc(Nc5ccc(F)c(Cl)c5)c4cc3NC(=O)C=C)CC2)C([2H])([2H])C([2H])([2H])N(c2cc3c(c(F)c2F)C(=O)N(C2CCC(=O)NC2=O)C3=O)C1([2H])[2H]] | N-(4-((3-chloro-4-fluorophenyl)amino)-7-(3- (4-((4-(2-(2,6-dioxopiperidin-3-yl)-6,7- difluoro-1,3-dioxoisoindolin-5-yl)piperazin-1- yl-2,2,3,3,5,5,6,6-d₈)methyl)piperidin-1- yl)propoxy)quinazolin-6-yl)acrylamide |
| | 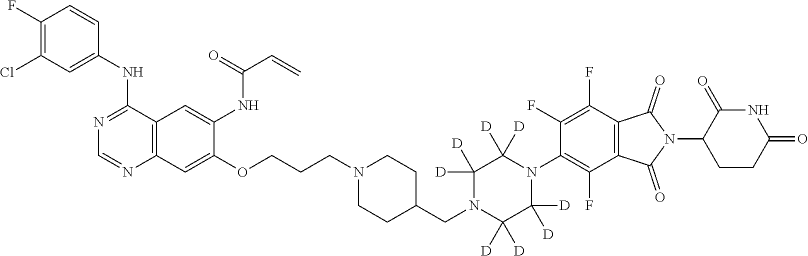 [SMILES: [2H]C1([2H])N(CC2CCN(CCCOc3cc4ncnc(Nc5ccc(F)c(Cl)c5)c4cc3NC(=O)C=C)CC2)C([2H])([2H])C([2H])([2H])N(c2c(F)c(F)c3c(c2F)C(=O)N(C2CCC(=O)NC2=O)C3=O)C1([2H])[2H]] | N-(4-((3-chloro-4-fluorophenyl)amino)-7-(3- (4-((4-(2-(2,6-dioxopiperidin-3-yl)-4,6,7- trifluoro-1,3-dioxoisoindolin-5-yl)piperazin-1- yl-2,2,3,3,5,5,6,6-d₈)methyl)piperidin-1- yl)propoxy)quinazolin-6-yl)acrylamide |
| | 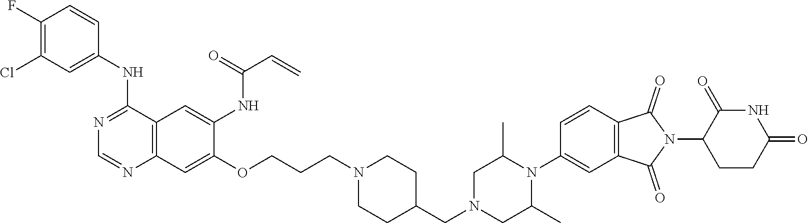 [SMILES: C=CC(=O)Nc1cc2c(Nc3ccc(F)c(Cl)c3)ncnc2cc1OCCCN1CCC(CN2CC(C)N(c3ccc4c(c3)C(=O)N(C3CCC(=O)NC3=O)C4=O)C(C)C2)CC1] | N-(4-((3-chloro-4-fluorophenyl)amino)-7-(3- (4-((4-(2-(2,6-dioxopiperidin-3-yl)-1,3- dioxoisoindolin-5-yl)-3,5-dimethylpiperazin-1- yl)methyl)piperidin-1-yl)propoxy)quinazolin-6- yl)acrylamide |
| | 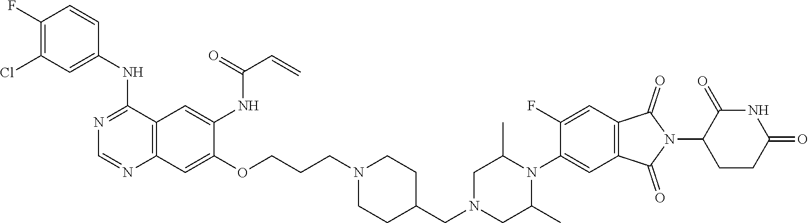 [SMILES: C=CC(=O)Nc1cc2c(Nc3ccc(F)c(Cl)c3)ncnc2cc1OCCCN1CCC(CN2CC(C)N(c3cc4c(cc3F)C(=O)N(C3CCC(=O)NC3=O)C4=O)C(C)C2)CC1] | N-(4-((3-chloro-4-fluorophenyl)amino)-7-(3- (4-((4-(2-(2,6-dioxopiperidin-3-yl)-6-fluoro- 1,3-dioxoisoindolin-5-yl)-3,5- dimethylpiperazin-1-yl)methyl)piperidin-1- yl)propoxy)quinazolin-6-yl)acrylamide |
| | 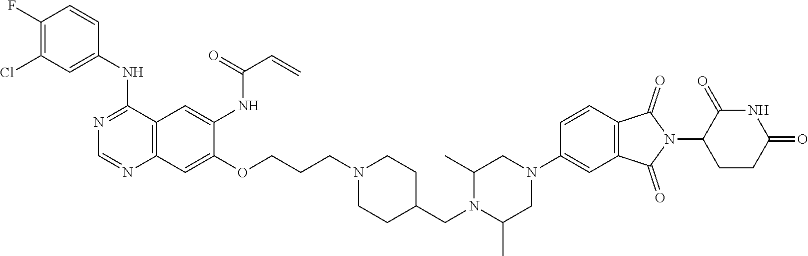 [SMILES: C=CC(=O)Nc1cc2c(Nc3ccc(F)c(Cl)c3)ncnc2cc1OCCCN1CCC(CN2C(C)CN(c3ccc4c(c3)C(=O)N(C3CCC(=O)NC3=O)C4=O)CC2C)CC1] | N-(4-((3-chloro-4-fluorophenyl)amino)-7-(3- (4-((4-(2-(2,6-dioxopiperidin-3-yl)-1,3- dioxoisoindolin-5-yl)-2,6-dimethylpiperazin-1- yl)methyl)piperidin-1-yl)propoxy)quinazolin-6- yl)acrylamide |
| | 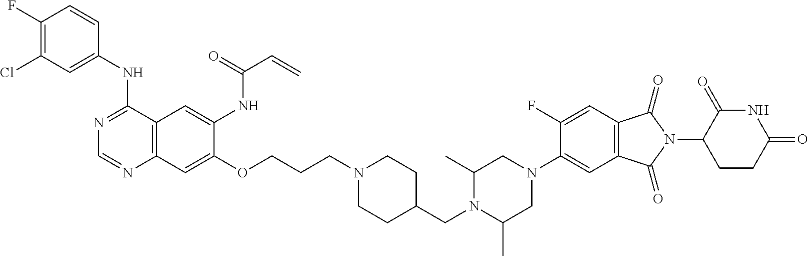 [SMILES: C=CC(=O)Nc1cc2c(Nc3ccc(F)c(Cl)c3)ncnc2cc1OCCCN1CCC(CN2C(C)CN(c3cc4c(cc3F)C(=O)N(C3CCC(=O)NC3=O)C4=O)CC2C)CC1] | N-(4-((3-chloro-4-fluorophenyl)amino)-7-(3- (4-((4-(2-(2,6-dioxopiperidin-3-yl)-6-fluoro- 1,3-dioxoisoindolin-5-yl)-2,6- dimethylpiperazin-1-yl)methyl)piperidin-1- yl)propoxy)quinazolin-6-yl)acrylamide |
| | 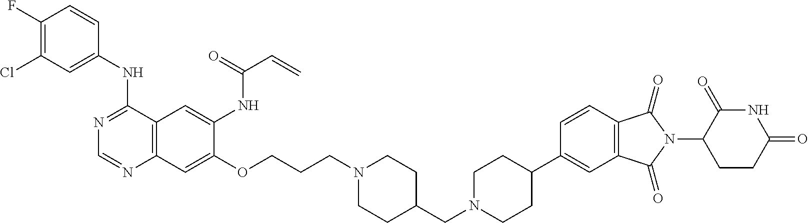 [SMILES: C=CC(=O)Nc1cc2c(Nc3ccc(F)c(Cl)c3)ncnc2cc1OCCCN1CCC(CN2CCC(c3ccc4c(c3)C(=O)N(C3CCC(=O)NC3=O)C4=O)CC2)CC1] | N-(4-((3-chloro-4-fluorophenyl)amino)-7-(3- (4-((4-(2-(2,6-dioxopiperidin-3-yl)-1,3- dioxoisoindolin-5-yl)piperidin-1- yl)methyl)piperidin-1-yl)propoxy)quinazolin-6- yl)acrylamide |
| | 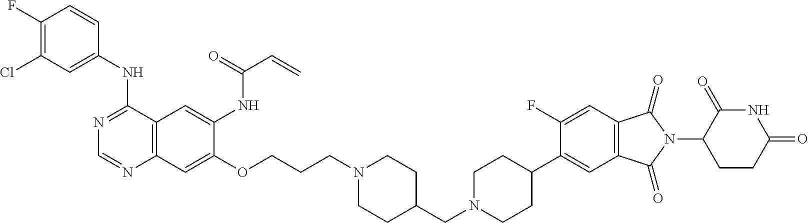 [SMILES: C=CC(=O)Nc1cc2c(Nc3ccc(F)c(Cl)c3)ncnc2cc1OCCCN1CCC(CN2CCC(c3cc4c(cc3F)C(=O)N(C3CCC(=O)NC3=O)C4=O)CC2)CC1] | N-(4-((3-chloro-4-fluorophenyl)amino)-7-(3- (4-((4-(2-(2,6-dioxopiperidin-3-yl)-6-fluoro- 1,3-dioxoisoindolin-5-yl)piperidin-1- yl)methyl)piperidin-1-yl)propoxy)quinazolin-6- yl)acrylamide |
| | 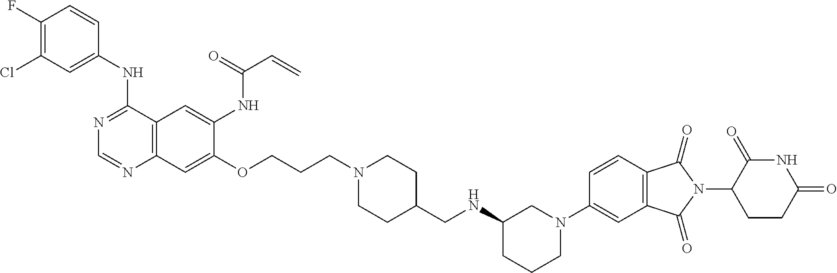 [SMILES: C=CC(=O)Nc1cc2c(Nc3ccc(F)c(Cl)c3)ncnc2cc1OCCCN1CCC(CN[C@@H]2CCCN(c3ccc4c(c3)C(=O)N(C3CCC(=O)NC3=O)C4=O)C2)CC1] | N-(4-((3-chloro-4-fluorophenyl)amino)-7-(3- (4-((((3R)-1-(2-(2,6-dioxopiperidin-3-yl)-1,3- dioxoisoindolin-5-yl)piperidin-3- yl)amino)methyl)piperidin-1- yl)propoxy)quinazolin-6-yl)acrylamide |
| | 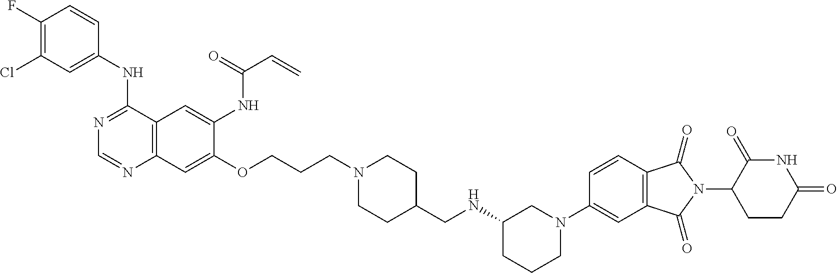 [SMILES: C=CC(=O)Nc1cc2c(Nc3ccc(F)c(Cl)c3)ncnc2cc1OCCCN1CCC(CN[C@H]2CCCN(c3ccc4c(c3)C(=O)N(C3CCC(=O)NC3=O)C4=O)C2)CC1] | N-(4-((3-chloro-4-fluorophenyl)amino)-7-(3- (4-((((3S)-1-(2-(2,6-dioxopiperidin-3-yl)-1,3- dioxoisoindolin-5-yl)piperidin-3- yl)amino)methyl)piperidin-1- yl)propoxy)quinazolin-6-yl)acrylamide |
| | 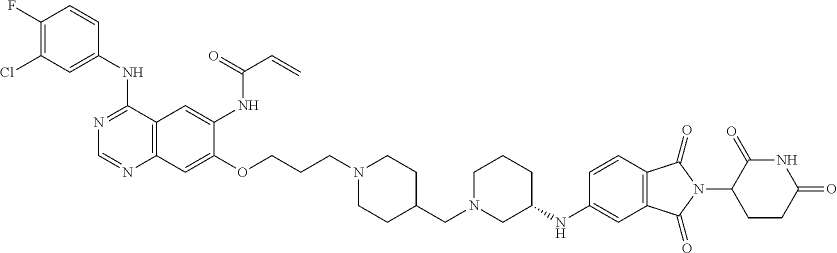 [SMILES: C=CC(=O)Nc1cc2c(Nc3ccc(F)c(Cl)c3)ncnc2cc1OCCCN1CCC(CN2CCC[C@H](Nc3ccc4c(c3)C(=O)N(C3CCC(=O)NC3=O)C4=O)C2)CC1] | N-(4-((3-chloro-4-fluorophenyl)amino)-7-(3- (4-(((3S)-3-((2-(2,6-dioxopiperidin-3-yl)-1,3- dioxoisoindolin-5-yl)amino)piperidin-1- yl)methyl)piperidin-1-yl)propoxy)quinazolin-6- yl)acrylamide |
| | 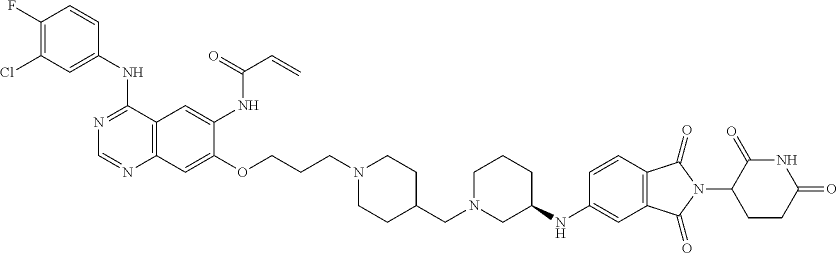 [SMILES: C=CC(=O)Nc1cc2c(Nc3ccc(F)c(Cl)c3)ncnc2cc1OCCCN1CCC(CN2CCC[C@@H](Nc3ccc4c(c3)C(=O)N(C3CCC(=O)NC3=O)C4=O)C2)CC1] | N-(4-((3-chloro-4-fluorophenyl)amino)-7-(3- (4-(((3R)-3-((2-(2,6-dioxopiperidin-3-yl)-1,3- dioxoisoindolin-5-yl)amino)piperidin-1- yl)methyl)piperidin-1-yl)propoxy)quinazolin-6- yl)acrylamide |
| | 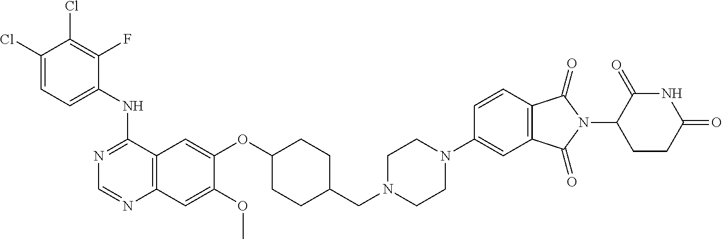 [SMILES: COc1cc2ncnc(Nc3ccc(Cl)c(Cl)c3F)c2cc1OC1CCC(CN2CCN(c3ccc4c(c3)C(=O)N(C3CCC(=O)NC3=O)C4=O)CC2)CC1] | 5-(4-((4-((4-((3,4-dichloro-2- fluorophenyl)amino)-7-methoxyquinazolin-6- yl)oxy)cyclohexyl)methyl)piperazin-1-yl)-2- (2,6-dioxopiperidin-3-yl)isoindoline-1,3-dione |
| | 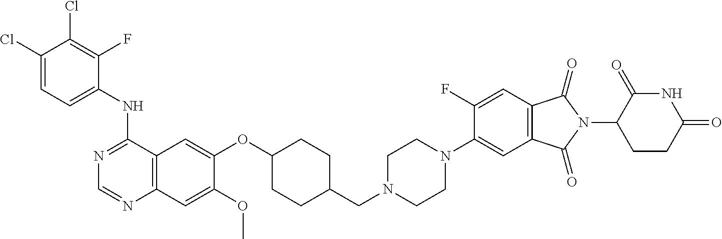 [SMILES: COc1cc2ncnc(Nc3ccc(Cl)c(Cl)c3F)c2cc1OC1CCC(CN2CCN(c3cc4c(cc3F)C(=O)N(C3CCC(=O)NC3=O)C4=O)CC2)CC1] | 5-(4-((4-((4-((3,4-dichloro-2- fluorophenyl)amino)-7-methoxyquinazolin-6- yl)oxy)cyclohexyl)methyl)piperazin-1-yl)-2- (2,6-dioxopiperidin-3-yl)-6-fluoroisoindoline- 1,3-dione |
| | 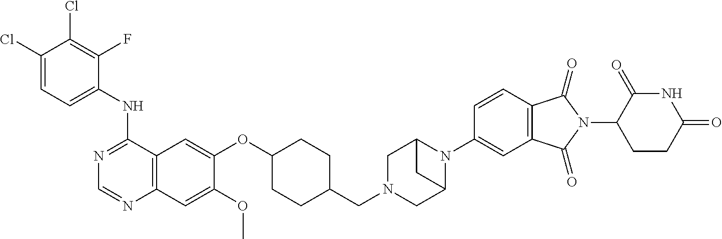 [SMILES: COc1cc2ncnc(Nc3ccc(Cl)c(Cl)c3F)c2cc1OC1CCC(CN2CC3CC(C2)N3c2ccc3c(c2)C(=O)N(C2CCC(=O)NC2=O)C3=O)CC1] | 5-(3-((4-((4-((3,4-dichloro-2- fluorophenyl)amino)-7-methoxyquinazolin-6- yl)oxy)cyclohexyl)methyl)-3,6- diazabicyclo[3.1.1]heptan-6-yl)-2-(2,6- dioxopiperidin-3-yl)isoindoline-1,3-dione |
| | 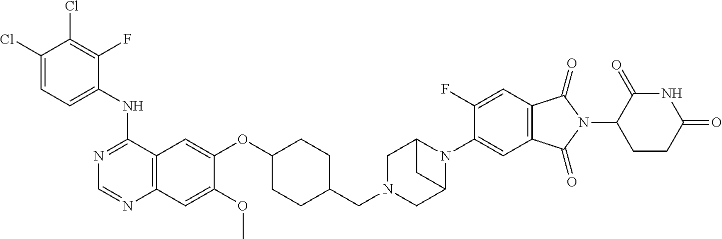 [SMILES: COc1cc2ncnc(Nc3ccc(Cl)c(Cl)c3F)c2cc1OC1CCC(CN2CC3CC(C2)N3c2cc3c(cc2F)C(=O)N(C2CCC(=O)NC2=O)C3=O)CC1] | 5-(3-((4-((4-((3,4-dichloro-2- fluorophenyl)amino)-7-methoxyquinazolin-6- yl)oxy)cyclohexyl)methyl)-3,6- diazabicyclo[3.1.1]heptan-6-yl)-2-(2,6- dioxopiperidin-3-yl)-6-fluoroisoindoline-1,3- dione |
| | 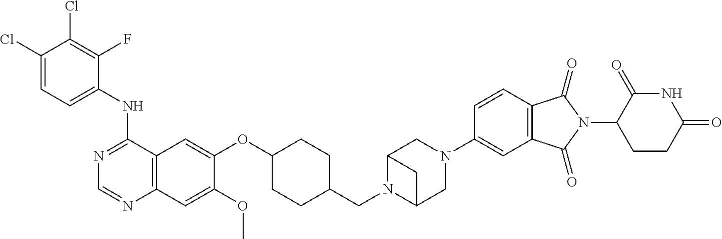 [SMILES: COc1cc2ncnc(Nc3ccc(Cl)c(Cl)c3F)c2cc1OC1CCC(CN2C3CC2CN(c2ccc4c(c2)C(=O)N(C2CCC(=O)NC2=O)C4=O)C3)CC1] | 5-(6-((4-((4-((3,4-dichloro-2- fluorophenyl)amino)-7-methoxyquinazolin-6- yl)oxy)cyclohexyl)methyl)-3,6- diazabicyclo[3.1.1]heptan-3-yl)-2-(2,6- dioxopiperidin-3-yl)isoindoline-1,3-dione |
| | 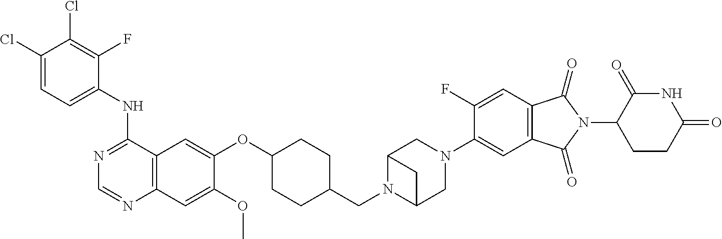 [SMILES: COc1cc2ncnc(Nc3ccc(Cl)c(Cl)c3F)c2cc1OC1CCC(CN2C3CC2CN(c2cc4c(cc2F)C(=O)N(C2CCC(=O)NC2=O)C4=O)C3)CC1] | 5-(6-((4-((4-((3,4-dichloro-2- fluorophenyl)amino)-7-methoxyquinazolin-6- yl)oxy)cyclohexyl)methyl)-3,6- diazabicyclo[3.1.1]heptan-3-yl)-2-(2,6- dioxopiperidin-3-yl)-6-fluoroisoindoline-1,3- dione |
| | 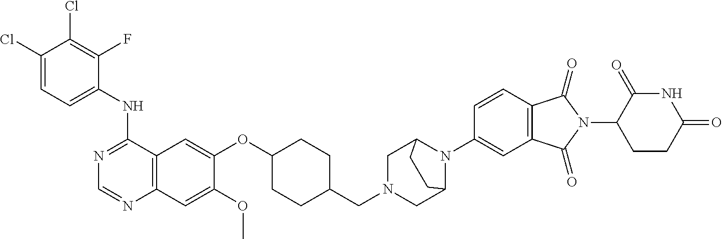 [SMILES: COc1cc2ncnc(Nc3ccc(Cl)c(Cl)c3F)c2cc1OC1CCC(CN2CC3CCC(C2)N3c2ccc3c(c2)C(=O)N(C2CCC(=O)NC2=O)C3=O)CC1] | 5-(3-((4-((4-((3,4-dichloro-2- fluorophenyl)amino)-7-methoxyquinazolin-6- yl)oxy)cyclohexyl)methyl)-3,8- diazabicyclo[3.2.1]octan-8-yl)-2-(2,6- dioxopiperidin-3-yl)isoindoline-1,3-dione |
| | 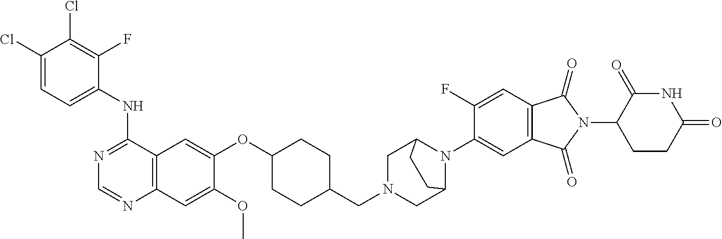 [SMILES: COc1cc2ncnc(Nc3ccc(Cl)c(Cl)c3F)c2cc1OC1CCC(CN2CC3CCC(C2)N3c2cc3c(cc2F)C(=O)N(C2CCC(=O)NC2=O)C3=O)CC1] | 5-(3-((4-((4-((3,4-dichloro-2- fluorophenyl)amino)-7-methoxyquinazolin-6- yl)oxy)cyclohexyl)methyl)-3,8- diazabicyclo[3.2.1]octan-8-yl)-2-(2,6- dioxopiperidin-3-yl)-6-fluoroisoindoline-1,3- dione |
| | 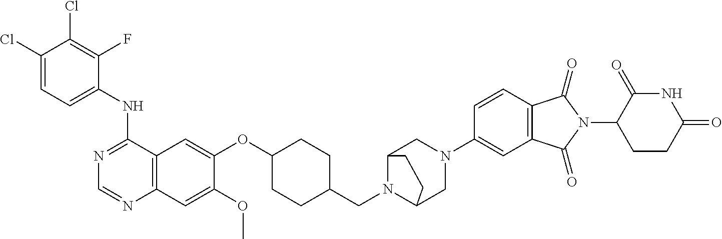 [SMILES: COc1cc2ncnc(Nc3ccc(Cl)c(Cl)c3F)c2cc1OC1CCC(CN2C3CCC2CN(c2ccc4c(c2)C(=O)N(C2CCC(=O)NC2=O)C4=O)C3)CC1] | 5-(8-((4-((4-((3,4-dichloro-2- fluorophenyl)amino)-7-methoxyquinazolin-6- yl)oxy)cyclohexyl)methyl)-3,8- diazabicyclo[3.2.1]octan-3-yl)-2-(2,6- dioxopiperidin-3-yl)isoindoline-1,3-dione |
| | 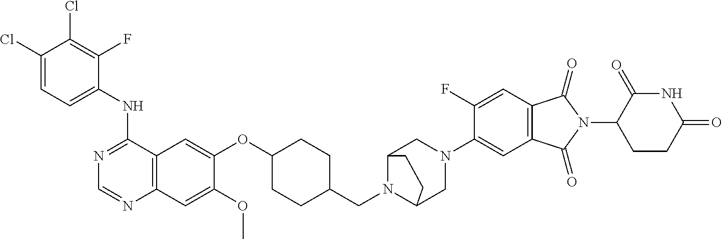 [SMILES: COc1cc2ncnc(Nc3ccc(Cl)c(Cl)c3F)c2cc1OC1CCC(CN2C3CCC2CN(c2cc4c(cc2F)C(=O)N(C2CCC(=O)NC2=O)C4=O)C3)CC1] | 5-(8-((4-((4-((3,4-dichloro-2- fluorophenyl)amino)-7-methoxyquinazolin-6- yl)oxy)cyclohexyl)methyl)-3,8- diazabicyclo[3.2.1]octan-3-yl)-2-(2,6- dioxopiperidin-3-yl)-6-fluoroisoindoline-1,3- dione |
| | 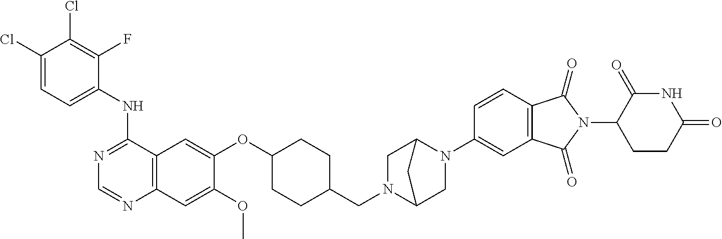 [SMILES: COc1cc2ncnc(Nc3ccc(Cl)c(Cl)c3F)c2cc1OC1CCC(CN2CC3CC2CN3c2ccc3c(c2)C(=O)N(C2CCC(=O)NC2=O)C3=O)CC1] | 5-(5-((4-((4-((3,4-dichloro-2- fluorophenyl)amino)-7-methoxyquinazolin-6- yl)oxy)cyclohexyl)methyl)-2,5- diazabicyclo[2.2.1]heptan-2-yl)-2-(2,6- dioxopiperidin-3-yl)isoindoline-1,3-dione |
| | 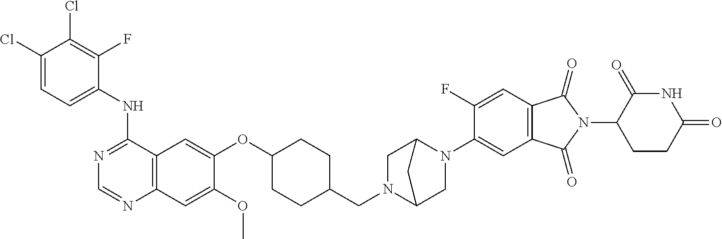 [SMILES: COc1cc2ncnc(Nc3ccc(Cl)c(Cl)c3F)c2cc1OC1CCC(CN2CC3CC2CN3c2cc3c(cc2F)C(=O)N(C2CCC(=O)NC2=O)C3=O)CC1] | 5-(5-((4-((4-((3,4-dichloro-2- fluorophenyl)amino)-7-methoxyquinazolin-6- yl)oxy)cyclohexyl)methyl)-2,5- diazabicyclo[2.2.1]heptan-2-yl)-2-(2,6- dioxopiperidin-3-yl)-6-fluoroisoindoline-1,3- dione |
| | 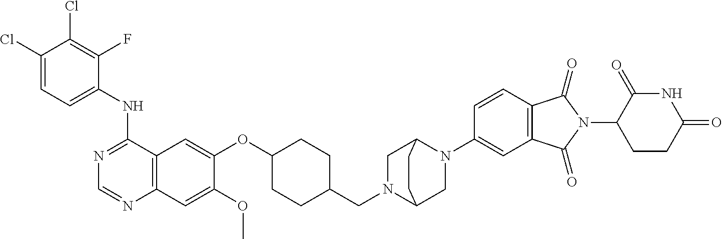 [SMILES: COc1cc2ncnc(Nc3ccc(Cl)c(Cl)c3F)c2cc1OC1CCC(CN2CC3CCC2CN3c2ccc3c(c2)C(=O)N(C2CCC(=O)NC2=O)C3=O)CC1] | 5-(5-((4-((4-((3,4-dichloro-2- fluorophenyl)amino)-7-methoxyquinazolin-6- yl)oxy)cyclohexyl)methyl)-2,5- diazabicyclo[2.2.2]octan-2-yl)-2-(2,6- dioxopiperidin-3-yl)isoindoline-1,3-dione |
| | 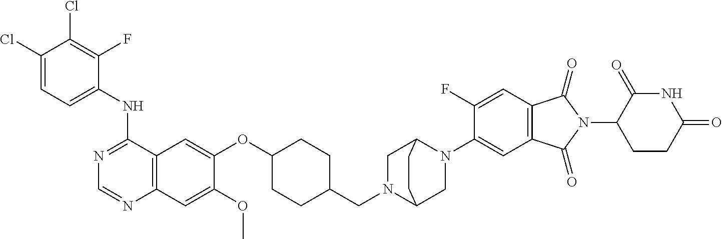 [SMILES: COc1cc2ncnc(Nc3ccc(Cl)c(Cl)c3F)c2cc1OC1CCC(CN2CC3CCC2CN3c2cc3c(cc2F)C(=O)N(C2CCC(=O)NC2=O)C3=O)CC1] | 5-(5-((4-((4-((3,4-dichloro-2- fluorophenyl)amino)-7-methoxyquinazolin-6- yl)oxy)cyclohexyl)methyl)-2,5- diazabicyclo[2.2.2]octan-2-yl)-2-(2,6- dioxopiperidin-3-yl)-6-fluoroisoindoline-1,3- dione |
| | 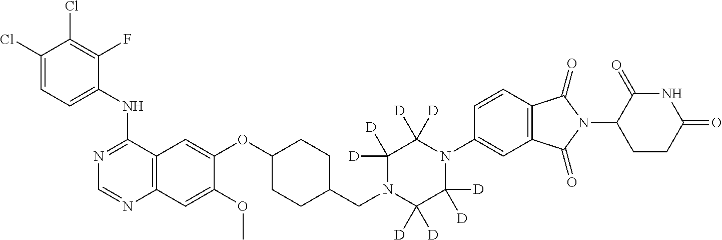 [SMILES: [2H]C1([2H])N(CC2CCC(Oc3cc4c(Nc5ccc(Cl)c(Cl)c5F)ncnc4cc3OC)CC2)C([2H])([2H])C([2H])([2H])N(c2ccc3c(c2)C(=O)N(C2CCC(=O)NC2=O)C3=O)C1([2H])[2H]] | 5-(4-((4-((4-((3,4-dichloro-2- fluorophenyl)amino)-7-methoxyquinazolin-6- yl)oxy)cyclohexyl)methyl)piperazin-1-yl- 2,2,3,3,5,5,6,6-d₈)-2-(2,6-dioxopiperidin-3- yl)isoindoline-1,3-dione |
| | 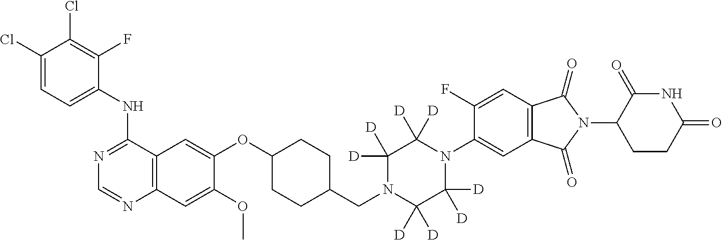 [SMILES: [2H]C1([2H])N(CC2CCC(Oc3cc4c(Nc5ccc(Cl)c(Cl)c5F)ncnc4cc3OC)CC2)C([2H])([2H])C([2H])([2H])N(c2cc3c(cc2F)C(=O)N(C2CCC(=O)NC2=O)C3=O)C1([2H])[2H]] | 5-(4-((4-((4-((3,4-dichloro-2- fluorophenyl)amino)-7-methoxyquinazolin-6- yl)oxy)cyclohexyl)methyl)piperazin-1-yl- 2,2,3,3,5,5,6,6-d₈)-2-(2,6-dioxopiperidin-3-yl)- 6-fluoroisoindoline-1,3-dione |
| | 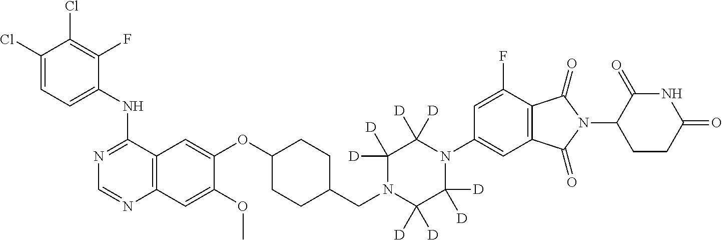 [SMILES: [2H]C1([2H])N(CC2CCC(Oc3cc4c(Nc5ccc(Cl)c(Cl)c5F)ncnc4cc3OC)CC2)C([2H])([2H])C([2H])([2H])N(c2cc(F)c3c(c2)C(=O)N(C2CCC(=O)NC2=O)C3=O)C1([2H])[2H]] | 6-(4-((4-((4-((3,4-dichloro-2- fluorophenyl)amino)-7-methoxyquinazolin-6- yl)oxy)cyclohexyl)methyl)piperazin-1-yl- 2,2,3,3,5,5,6,6-d₈)-2-(2,6-dioxopiperidin-3-yl)- 4-fluoroisoindoline-1,3-dione |
| | 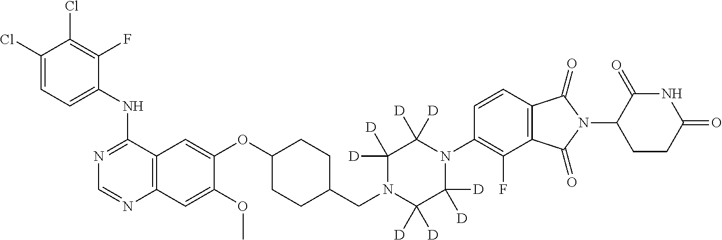 [SMILES: [2H]C1([2H])N(CC2CCC(Oc3cc4c(Nc5ccc(Cl)c(Cl)c5F)ncnc4cc3OC)CC2)C([2H])([2H])C([2H])([2H])N(c2ccc3c(c2F)C(=O)N(C2CCC(=O)NC2=O)C3=O)C1([2H])[2H]] | 5-(4-((4-((4-((3,4-dichloro-2- fluorophenyl)amino)-7-methoxyquinazolin-6- yl)oxy)cyclohexyl)methyl)piperazin-1-yl- 2,2,3,3,5,5,6,6-d₈)-2-(2,6-dioxopiperidin-3-yl)- 4-fluoroisoindoline-1,3-dione |
| | 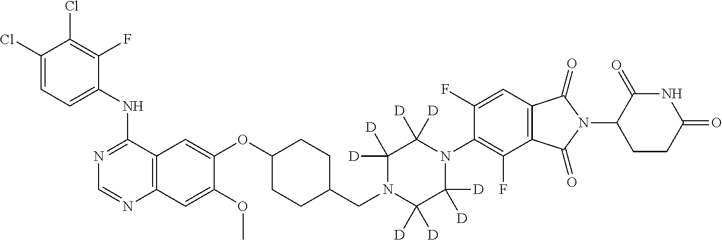 [SMILES: [2H]C1([2H])N(CC2CCC(Oc3cc4c(Nc5ccc(Cl)c(Cl)c5F)ncnc4cc3OC)CC2)C([2H])([2H])C([2H])([2H])N(c2c(F)cc3c(c2F)C(=O)N(C2CCC(=O)NC2=O)C3=O)C1([2H])[2H]] | 5-(4-((4-((4-((3,4-dichloro-2- fluorophenyl)amino)-7-methoxyquinazolin-6- yl)oxy)cyclohexyl)methyl)piperazin-1-yl- 2,2,3,3,5,5,6,6-d₈)-2-(2,6-dioxopiperidin-3-yl)- 4,6-difluoroisoindoline-1,3-dione |
| | 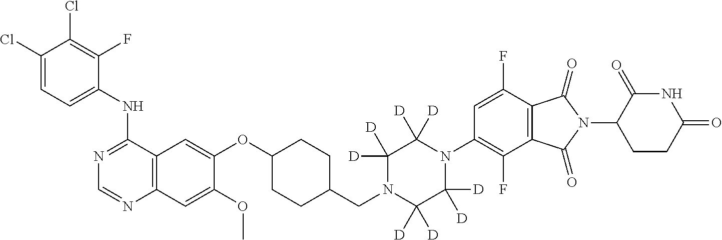 [SMILES: [2H]C1([2H])N(CC2CCC(Oc3cc4c(Nc5ccc(Cl)c(Cl)c5F)ncnc4cc3OC)CC2)C([2H])([2H])C([2H])([2H])N(c2cc(F)c3c(c2F)C(=O)N(C2CCC(=O)NC2=O)C3=O)C1([2H])[2H]] | 5-(4-((4-((4-((3,4-dichloro-2- fluorophenyl)amino)-7-methoxyquinazolin-6- yl)oxy)cyclohexyl)methyl)piperazin-1-yl- 2,2,3,3,5,5,6,6-d₈)-2-(2,6-dioxopiperidin-3-yl)- 4,7-difluoroisoindoline-1,3-dione |
| | 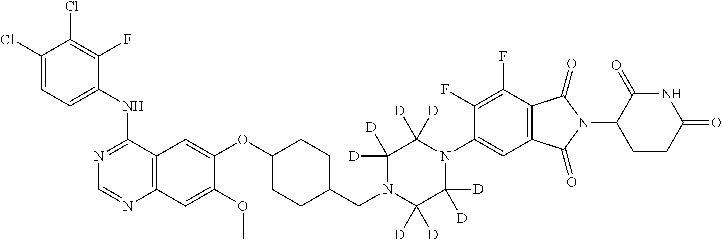 [SMILES: [2H]C1([2H])N(CC2CCC(Oc3cc4c(Nc5ccc(Cl)c(Cl)c5F)ncnc4cc3OC)CC2)C([2H])([2H])C([2H])([2H])N(c2cc3c(c(F)c2F)C(=O)N(C2CCC(=O)NC2=O)C3=O)C1([2H])[2H]] | 6-(4-((4-((4-((3,4-dichloro-2- fluorophenyl)amino)-7-methoxyquinazolin-6- yl)oxy)cyclohexyl)methyl)piperazin-1-yl- 2,2,3,3,5,5,6,6-d₈)-2-(2,6-dioxopiperidin-3-yl)- 4,5-difluoroisoindoline-1,3-dione |
| | 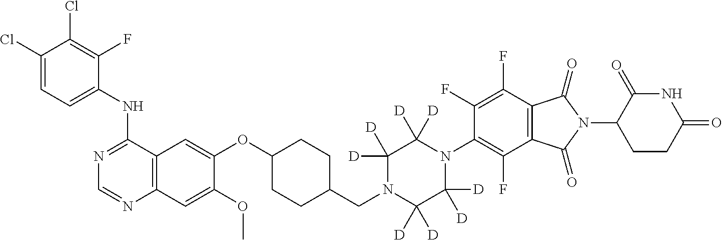 [SMILES: [2H]C1([2H])N(CC2CCC(Oc3cc4c(Nc5ccc(Cl)c(Cl)c5F)ncnc4cc3OC)CC2)C([2H])([2H])C([2H])([2H])N(c2c(F)c(F)c3c(c2F)C(=O)N(C2CCC(=O)NC2=O)C3=O)C1([2H])[2H]] | 5-(4-((4-((4-((3,4-dichloro-2- fluorophenyl)amino)-7-methoxyquinazolin-6- yl)oxy)cyclohexyl)methyl)piperazin-1-yl- 2,2,3,3,5,5,6,6-d₈)-2-(2,6-dioxopiperidin-3-yl)- 4,6,7-trifluoroisoindoline-1,3-dione |
| | 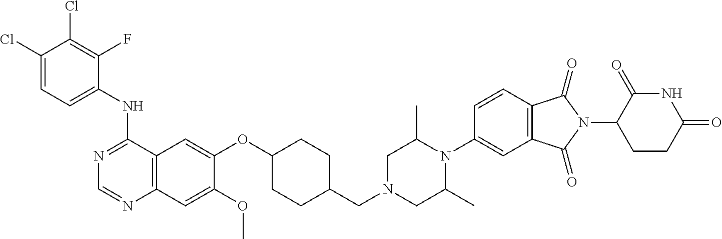 [SMILES: COc1cc2ncnc(Nc3ccc(Cl)c(Cl)c3F)c2cc1OC1CCC(CN2CC(C)N(c3ccc4c(c3)C(=O)N(C3CCC(=O)NC3=O)C4=O)C(C)C2)CC1] | 5-(4-((4-((4-((3,4-dichloro-2- fluorophenyl)amino)-7-methoxyquinazolin-6- yl)oxy)cyclohexyl)methyl)-2,6- dimethylpiperazin-1-yl)-2-(2,6-dioxopiperidin- 3-yl)isoindoline-1,3-dione |
| | 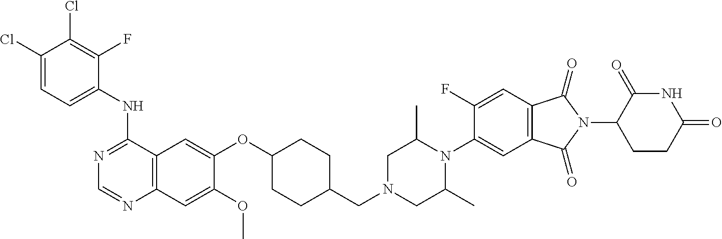 [SMILES: COc1cc2ncnc(Nc3ccc(Cl)c(Cl)c3F)c2cc1OC1CCC(CN2CC(C)N(c3cc4c(cc3F)C(=O)N(C3CCC(=O)NC3=O)C4=O)C(C)C2)CC1] | 5-(4-((4-((4-((3,4-dichloro-2- fluorophenyl)amino)-7-methoxyquinazolin-6- yl)oxy)cyclohexyl)methyl)-2,6- dimethylpiperazin-1-yl)-2-(2,6-dioxopiperidin- 3-yl)-6-fluoroisoindoline-1,3-dione |
| | 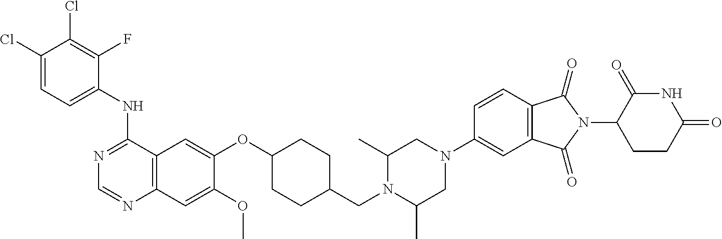 [SMILES: COc1cc2ncnc(Nc3ccc(Cl)c(Cl)c3F)c2cc1OC1CCC(CN2C(C)CN(c3ccc4c(c3)C(=O)N(C3CCC(=O)NC3=O)C4=O)CC2C)CC1] | 5-(4-((4-((4-((3,4-dichloro-2- fluorophenyl)amino)-7-methoxyquinazolin-6- yl)oxy)cyclohexyl)methyl)-3,5- dimethylpiperazin-1-yl)-2-(2,6-dioxopiperidin- 3-yl)isoindoline-1,3-dione |
| | 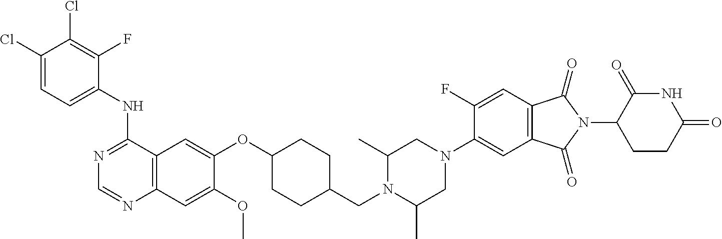 [SMILES: COc1cc2ncnc(Nc3ccc(Cl)c(Cl)c3F)c2cc1OC1CCC(CN2C(C)CN(c3cc4c(cc3F)C(=O)N(C3CCC(=O)NC3=O)C4=O)CC2C)CC1] | 5-(4-((4-((4-((3,4-dichloro-2- fluorophenyl)amino)-7-methoxyquinazolin-6- yl)oxy)cyclohexyl)methyl)-3,5- dimethylpiperazin-1-yl)-2-(2,6-dioxopiperidin- 3-yl)-6-fluoroisoindoline-1,3-dione |
| | 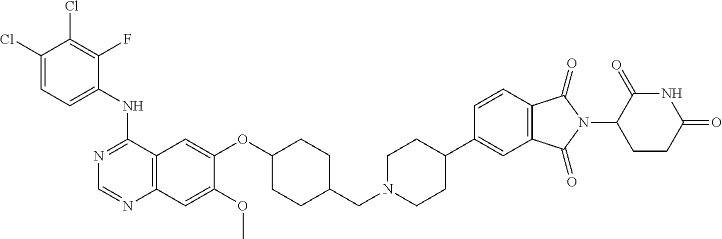 [SMILES: COc1cc2ncnc(Nc3ccc(Cl)c(Cl)c3F)c2cc1OC1CCC(CN2CCC(c3ccc4c(c3)C(=O)N(C3CCC(=O)NC3=O)C4=O)CC2)CC1] | 5-(1-((4-((4-((3,4-dichloro-2- fluorophenyl)amino)-7-methoxyquinazolin-6- yl)oxy)cyclohexyl)methyl)piperidin-4-yl)-2- (2,6-dioxopiperidin-3-yl)isoindoline-1,3-dione |
| | 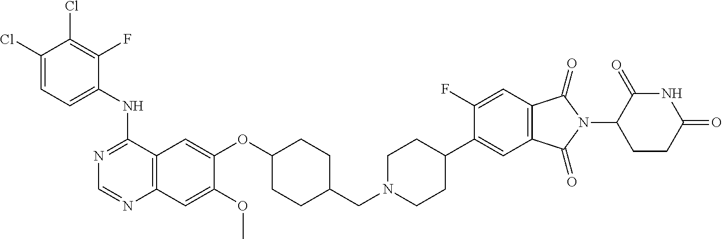 [SMILES: COc1cc2ncnc(Nc3ccc(Cl)c(Cl)c3F)c2cc1OC1CCC(CN2CCC(c3cc4c(cc3F)C(=O)N(C3CCC(=O)NC3=O)C4=O)CC2)CC1] | 5-(1-((4-((4-((3,4-dichloro-2- fluorophenyl)amino)-7-methoxyquinazolin-6- yl)oxy)cyclohexyl)methyl)piperidin-4-yl)-2- (2,6-dioxopiperidin-3-yl)-6-fluoroisoindoline- 1,3-dione |
| | 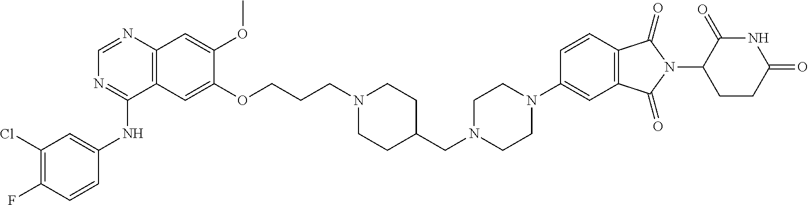 [SMILES: COc1cc2ncnc(Nc3ccc(F)c(Cl)c3)c2cc1OCCCN1CCC(CN2CCN(c3ccc4c(c3)C(=O)N(C3CCC(=O)NC3=O)C4=O)CC2)CC1] | 5-(4-((1-(3-((4-((3-chloro-4- fluorophenyl)amino)-7-methoxyquinazolin-6- yl)oxy)propyl)piperidin-4-yl)methyl)piperazin- 1-yl)-2-(2,6-dioxopiperidin-3-yl)isoindoline- 1,3-dione |
| | 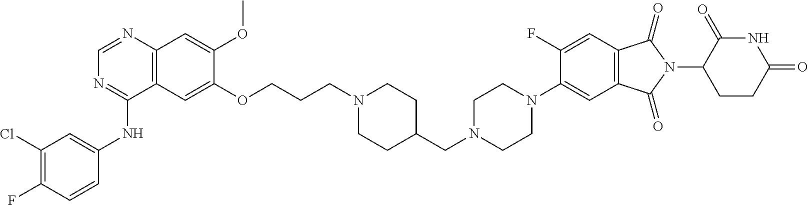 [SMILES: COc1cc2ncnc(Nc3ccc(F)c(Cl)c3)c2cc1OCCCN1CCC(CN2CCN(c3cc4c(cc3F)C(=O)N(C3CCC(=O)NC3=O)C4=O)CC2)CC1] | 5-(4-((1-(3-((4-((3-chloro-4- fluorophenyl)amino)-7-methoxyquinazolin-6- yl)oxy)propyl)piperidin-4-yl)methyl)piperazin- 1-yl)-2-(2,6-dioxopiperidin-3-yl)-6- fluoroisoindoline-1,3-dione |
| | 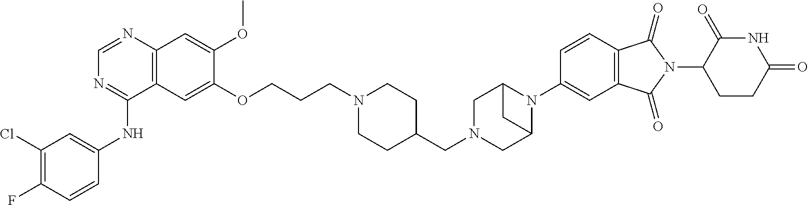 [SMILES: COc1cc2ncnc(Nc3ccc(F)c(Cl)c3)c2cc1OCCCN1CCC(CN2CC3CC(C2)N3c2ccc3c(c2)C(=O)N(C2CCC(=O)NC2=O)C3=O)CC1] | 5-(3-((1-(3-((4-((3-chloro-4- fluorophenyl)amino)-7-methoxyquinazolin-6- yl)oxy)propyl)piperidin-4-yl)methyl)-3,6- diazabicyclo[3.1.1]heptan-6-yl)-2-(2,6- dioxopiperidin-3-yl)isoindoline-1,3-dione |
| | 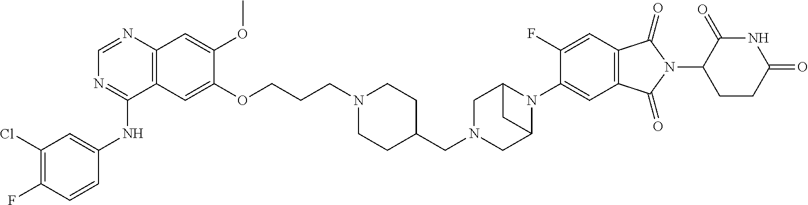 [SMILES: COc1cc2ncnc(Nc3ccc(F)c(Cl)c3)c2cc1OCCCN1CCC(CN2CC3CC(C2)N3c2cc3c(cc2F)C(=O)N(C2CCC(=O)NC2=O)C3=O)CC1] | 5-(3-((1-(3-((4-((3-chloro-4- fluorophenyl)amino)-7-methoxyquinazolin-6- yl)oxy)propyl)piperidin-4-yl)methyl)-3,6- diazabicyclo[3.1.1]heptan-6-yl)-2-(2,6- dioxopiperidin-3-yl)-6-fluoroisoindoline-1,3- dione |
| | 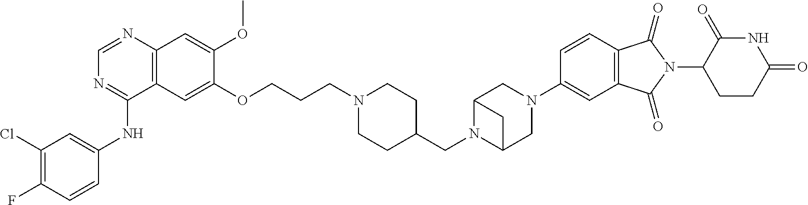 [SMILES: COc1cc2ncnc(Nc3ccc(F)c(Cl)c3)c2cc1OCCCN1CCC(CN2C3CC2CN(c2ccc4c(c2)C(=O)N(C2CCC(=O)NC2=O)C4=O)C3)CC1] | 5-(6-((1-(3-((4-((3-chloro-4- fluorophenyl)amino)-7-methoxyquinazolin-6- yl)oxy)propyl)piperidin-4-yl)methyl)-3,6- diazabicyclo[3.1.1]heptan-3-yl)-2-(2,6- dioxopiperidin-3-yl)isoindoline-1,3-dione |
| | 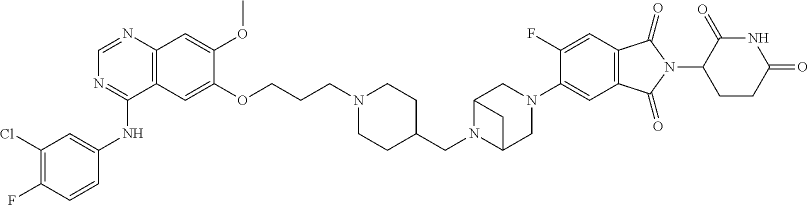 [SMILES: COc1cc2ncnc(Nc3ccc(F)c(Cl)c3)c2cc1OCCCN1CCC(CN2C3CC2CN(c2cc4c(cc2F)C(=O)N(C2CCC(=O)NC2=O)C4=O)C3)CC1] | 5-(6-((1-(3-((4-((3-chloro-4- fluorophenyl)amino)-7-methoxyquinazolin-6- yl)oxy)propyl)piperidin-4-yl)methyl)-3,6- diazabicyclo[3.1.1]heptan-3-yl)-2-(2,6- dioxopiperidin-3-yl)-6-fluoroisoindoline-1,3- dione |
| | 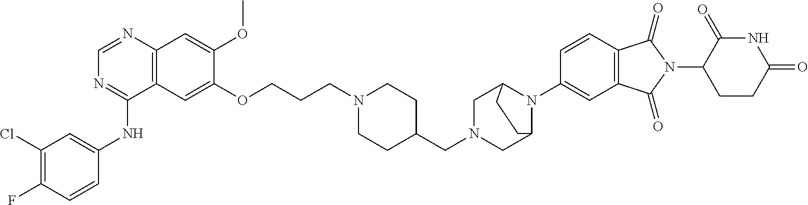 [SMILES: COc1cc2ncnc(Nc3ccc(F)c(Cl)c3)c2cc1OCCCN1CCC(CN2CC3CCC(C2)N3c2ccc3c(c2)C(=O)N(C2CCC(=O)NC2=O)C3=O)CC1] | 5-(3-((1-(3-((4-((3-chloro-4- fluorophenyl)amino)-7-methoxyquinazolin-6- yl)oxy)propyl)piperidin-4-yl)methyl)-3,8- diazabicyclo[3.2.1]octan-8-yl)-2-(2,6- dioxopiperidin-3-yl)isoindoline-1,3-dione |
| | 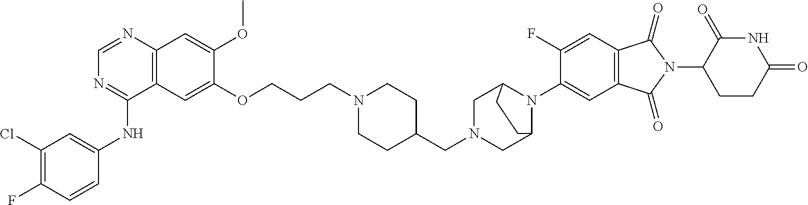 [SMILES: COc1cc2ncnc(Nc3ccc(F)c(Cl)c3)c2cc1OCCCN1CCC(CN2CC3CCC(C2)N3c2cc3c(cc2F)C(=O)N(C2CCC(=O)NC2=O)C3=O)CC1] | 5-(3-((1-(3-((4-((3-chloro-4- fluorophenyl)amino)-7-methoxyquinazolin-6- yl)oxy)propyl)piperidin-4-yl)methyl)-3,8- diazabicyclo[3.2.1]octan-8-yl)-2-(2,6- dioxopiperidin-3-yl)-6-fluoroisoindoline-1,3- dione |
| | 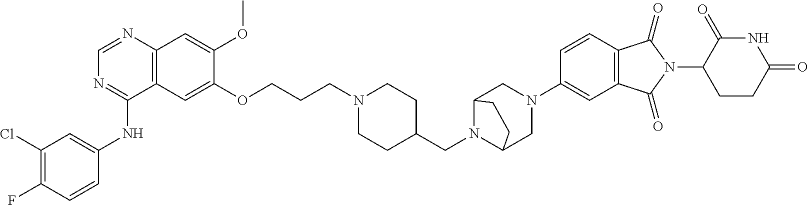 [SMILES: COc1cc2ncnc(Nc3ccc(F)c(Cl)c3)c2cc1OCCCN1CCC(CN2C3CCC2CN(c2ccc4c(c2)C(=O)N(C2CCC(=O)NC2=O)C4=O)C3)CC1] | 5-(8-((1-(3-((4-((3-chloro-4- fluorophenyl)amino)-7-methoxyquinazolin-6- yl)oxy)propyl)piperidin-4-yl)methyl)-3,8- diazabicyclo[3.2.1]octan-3-yl)-2-(2,6- dioxopiperidin-3-yl)isoindoline-1,3-dione |
| | 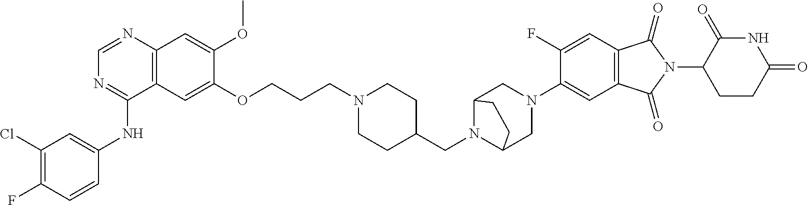 [SMILES: COc1cc2ncnc(Nc3ccc(F)c(Cl)c3)c2cc1OCCCN1CCC(CN2C3CCC2CN(c2cc4c(cc2F)C(=O)N(C2CCC(=O)NC2=O)C4=O)C3)CC1] | 5-(8-((1-(3-((4-((3-chloro-4- fluorophenyl)amino)-7-methoxyquinazolin-6- yl)oxy)propyl)piperidin-4-yl)methyl)-3,8- diazabicyclo[3.2.1]octan-3-yl)-2-(2,6- dioxopiperidin-3-yl)-6-fluoroisoindoline-1,3- dione |
| | 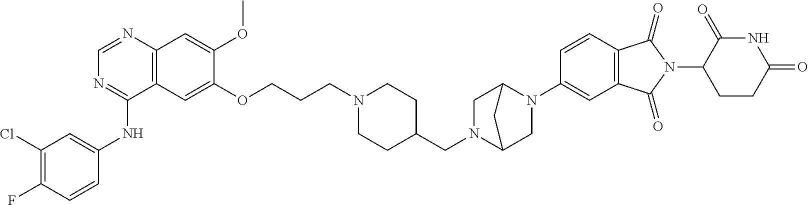 [SMILES: COc1cc2ncnc(Nc3ccc(F)c(Cl)c3)c2cc1OCCCN1CCC(CN2CC3CC2CN3c2ccc3c(c2)C(=O)N(C2CCC(=O)NC2=O)C3=O)CC1] | 5-(5-((1-(3-((4-((3-chloro-4- fluorophenyl)amino)-7-methoxyquinazolin-6- yl)oxy)propyl)piperidin-4-yl)methyl)-2,5- diazabicyclo[2.2.1]heptan-2-yl)-2-(2,6- dioxopiperidin-3-yl)isoindoline-1,3-dione |
| | 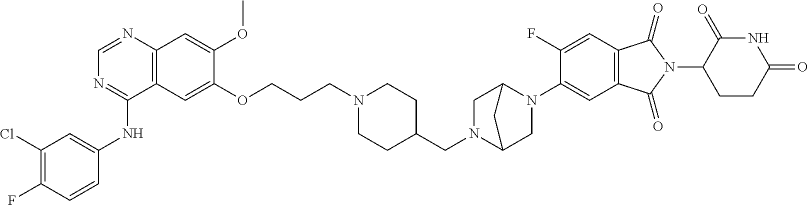 [SMILES: COc1cc2ncnc(Nc3ccc(F)c(Cl)c3)c2cc1OCCCN1CCC(CN2CC3CC2CN3c2cc3c(cc2F)C(=O)N(C2CCC(=O)NC2=O)C3=O)CC1] | 5-(5-((1-(3-((4-((3-chloro-4- fluorophenyl)amino)-7-methoxyquinazolin-6- yl)oxy)propyl)piperidin-4-yl)methyl)-2,5- diazabicyclo[2.2.1]heptan-2-yl)-2-(2,6- dioxopiperidin-3-yl)-6-fluoroisoindoline-1,3- dione |
| | 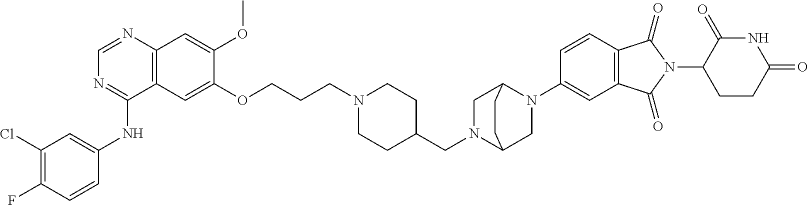 [SMILES: COc1cc2ncnc(Nc3ccc(F)c(Cl)c3)c2cc1OCCCN1CCC(CN2CC3CCC2CN3c2ccc3c(c2)C(=O)N(C2CCC(=O)NC2=O)C3=O)CC1] | 5-(5-((1-(3-((4-((3-chloro-4- fluorophenyl)amino)-7-methoxyquinazolin-6- yl)oxy)propyl)piperidin-4-yl)methyl)-2,5- diazabicyclo[2.2.2]octan-2-yl)-2-(2,6- dioxopiperidin-3-yl)isoindoline-1,3-dione |
| | 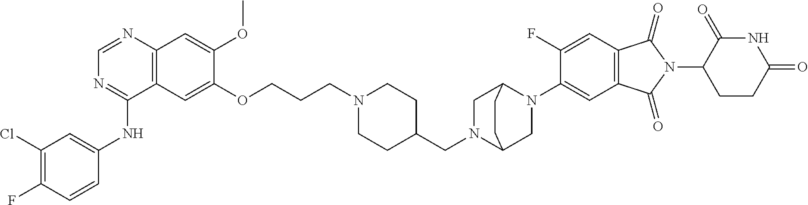 [SMILES: COc1cc2ncnc(Nc3ccc(F)c(Cl)c3)c2cc1OCCCN1CCC(CN2CC3CCC2CN3c2cc3c(cc2F)C(=O)N(C2CCC(=O)NC2=O)C3=O)CC1] | 5-(5-((1-(3-((4-((3-chloro-4- fluorophenyl)amino)-7-methoxyquinazolin-6- yl)oxy)propyl)piperidin-4-yl)methyl)-2,5- diazabicyclo[2.2.2]octan-2-yl)-2-(2,6- dioxopiperidin-3-yl)-6-fluoroisoindoline-1,3- dione |
| | 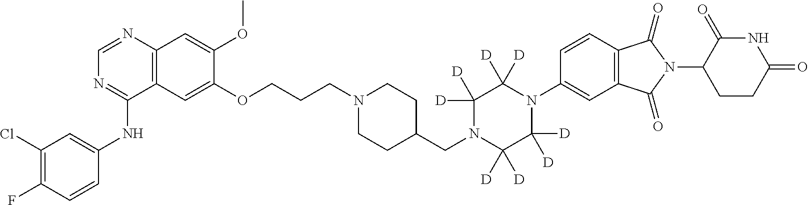 [SMILES: [2H]C1([2H])N(CC2CCN(CCCOc3cc4c(Nc5ccc(F)c(Cl)c5)ncnc4cc3OC)CC2)C([2H])([2H])C([2H])([2H])N(c2ccc3c(c2)C(=O)N(C2CCC(=O)NC2=O)C3=O)C1([2H])[2H]] | 5-(4-((1-(3-((4-((3-chloro-4- fluorophenyl)amino)-7-methoxyquinazolin-6- yl)oxy)propyl)piperidin-4-yl)methyl)piperazin- 1-yl-2,2,3,3,5,5,6,6-d₈)-2-(2,6-dioxopiperidin- 3-yl)isoindoline-1,3-dione |
| | 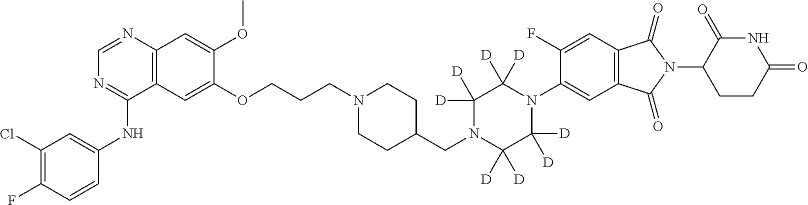 [SMILES: [2H]C1([2H])N(CC2CCN(CCCOc3cc4c(Nc5ccc(F)c(Cl)c5)ncnc4cc3OC)CC2)C([2H])([2H])C([2H])([2H])N(c2cc3c(cc2F)C(=O)N(C2CCC(=O)NC2=O)C3=O)C1([2H])[2H]] | 5-(4-((1-(3-((4-((3-chloro-4- fluorophenyl)amino)-7-methoxyquinazolin-6- yl)oxy)propyl)piperidin-4-yl)methyl)piperazin- 1-yl-2,2,3,3,5,5,6,6-d₈)-2-(2,6-dioxopiperidin- 3-yl)-6-fluoroisoindoline-1,3-dione |
| | 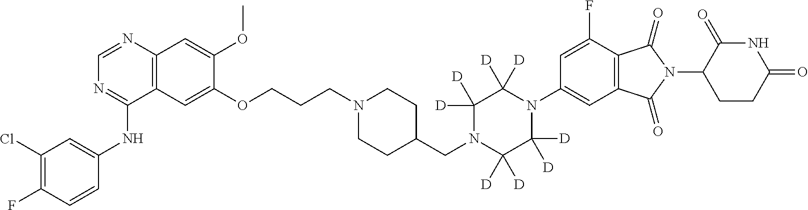 [SMILES: [2H]C1([2H])N(CC2CCN(CCCOc3cc4c(Nc5ccc(F)c(Cl)c5)ncnc4cc3OC)CC2)C([2H])([2H])C([2H])([2H])N(c2cc(F)c3c(c2)C(=O)N(C2CCC(=O)NC2=O)C3=O)C1([2H])[2H]] | 6-(4-((1-(3-((4-((3-chloro-4- fluorophenyl)amino)-7-methoxyquinazolin-6- yl)oxy)propyl)piperidin-4-yl)methyl)piperazin- 1-yl-2,2,3,3,5,5,6,6-d₈)-2-(2,6-dioxopiperidin- 3-yl)-4-fluoroisoindoline-1,3-dione |
| | 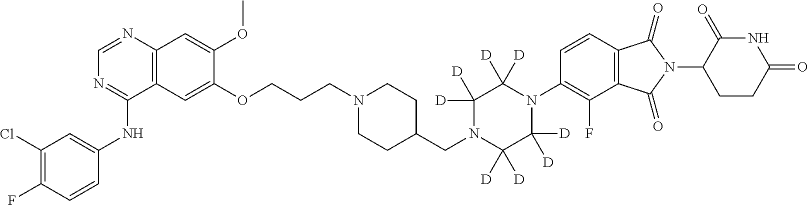 [SMILES: [2H]C1([2H])N(CC2CCN(CCCOc3cc4c(Nc5ccc(F)c(Cl)c5)ncnc4cc3OC)CC2)C([2H])([2H])C([2H])([2H])N(c2ccc3c(c2F)C(=O)N(C2CCC(=O)NC2=O)C3=O)C1([2H])[2H]] | 5-(4-((1-(3-((4-((3-chloro-4- fluorophenyl)amino)-7-methoxyquinazolin-6- yl)oxy)propyl)piperidin-4-yl)methyl)piperazin- 1-yl-2,2,3,3,5,5,6,6-d₈)-2-(2,6-dioxopiperidin- 3-yl)-4-fluoroisoindoline-1,3-dione |
| | 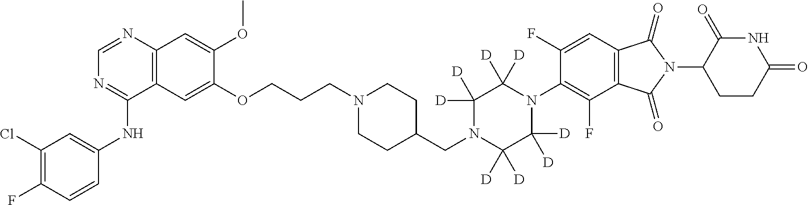 [SMILES: [2H]C1([2H])N(CC2CCN(CCCOc3cc4c(Nc5ccc(F)c(Cl)c5)ncnc4cc3OC)CC2)C([2H])([2H])C([2H])([2H])N(c2c(F)cc3c(c2F)C(=O)N(C2CCC(=O)NC2=O)C3=O)C1([2H])[2H]] | 5-(4-((1-(3-((4-((3-chloro-4- fluorophenyl)amino)-7-methoxyquinazolin-6- yl)oxy)propyl)piperidin-4-yl)methyl)piperazin- 1-yl-2,2,3,3,5,5,6,6-d₈)-2-(2,6-dioxopiperidin- 3-yl)-4,6-difluoroisoindoline-1,3-dione |
| | 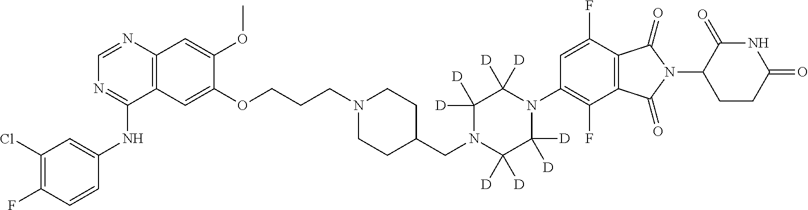 [SMILES: [2H]C1([2H])N(CC2CCN(CCCOc3cc4c(Nc5ccc(F)c(Cl)c5)ncnc4cc3OC)CC2)C([2H])([2H])C([2H])([2H])N(c2cc(F)c3c(c2F)C(=O)N(C2CCC(=O)NC2=O)C3=O)C1([2H])[2H]] | 5-(4-((1-(3-((4-((3-chloro-4- fluorophenyl)amino)-7-methoxyquinazolin-6- yl)oxy)propyl)piperidin-4-yl)methyl)piperazin- 1-yl-2,2,3,3,5,5,6,6-d₈)-2-(2,6-dioxopiperidin- 3-yl)-4,7-difluoroisoindoline-1,3-dione |
| | 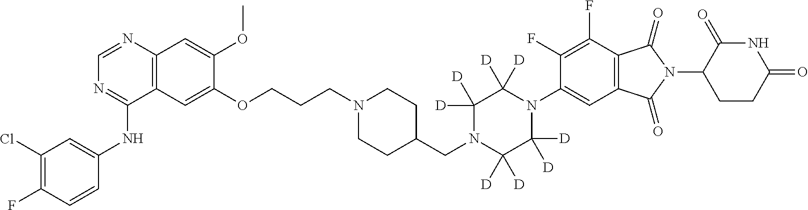 [SMILES: [2H]C1([2H])N(CC2CCN(CCCOc3cc4c(Nc5ccc(F)c(Cl)c5)ncnc4cc3OC)CC2)C([2H])([2H])C([2H])([2H])N(c2cc3c(c(F)c2F)C(=O)N(C2CCC(=O)NC2=O)C3=O)C1([2H])[2H]] | 6-(4-((1-(3-((4-((3-chloro-4- fluorophenyl)amino)-7-methoxyquinazolin-6- yl)oxy)propyl)piperidin-4-yl)methyl)piperazin- 1-yl-2,2,3,3,5,5,6,6-d₈)-2-(2,6-dioxopiperidin- 3-yl)-4,5-difluoroisoindoline-1,3-dione |
| | 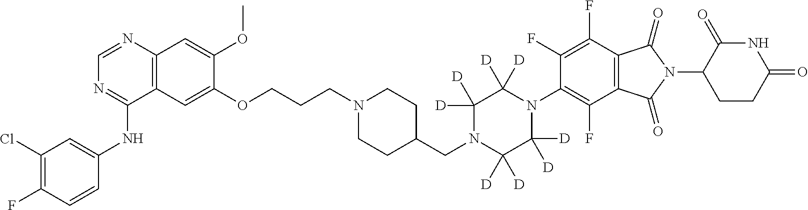 [SMILES: [2H]C1([2H])N(CC2CCN(CCCOc3cc4c(Nc5ccc(F)c(Cl)c5)ncnc4cc3OC)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(3-((4-((3-chloro-4- fluorophenyl)amino)-7-methoxyquinazolin-6- yl)oxy)propyl)piperidin-4-yl)methyl)piperazin- 1-yl-2,2,3,3,5,5,6,6-d₈)-2-(2,6-dioxopiperidin- 3-yl)-4,6,7-trifluoroisoindoline-1,3-dione |
| | 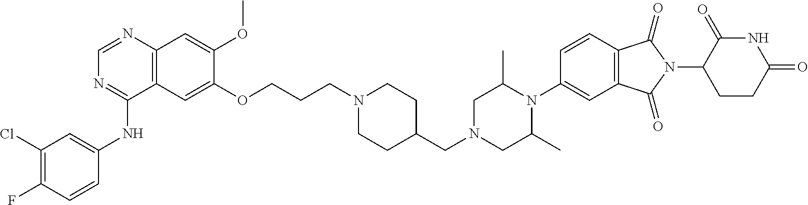 [SMILES: COc1cc2ncnc(Nc3ccc(F)c(Cl)c3)c2cc1OCCCN1CCC(CN2CC(C)N(c3ccc4c(c3)C(=O)N(C3CCC(=O)NC3=O)C4=O)C(C)C2)CC1] | 5-(4-((1-(3-((4-((3-chloro-4- fluorophenyl)amino)-7-methoxyquinazolin-6- yl)oxy)propyl)piperidin-4-yl)methyl)-2,6- dimethylpiperazin-1-yl)-2-(2,6-dioxopiperidin- 3-yl)isoindoline-1,3-dione |
| | 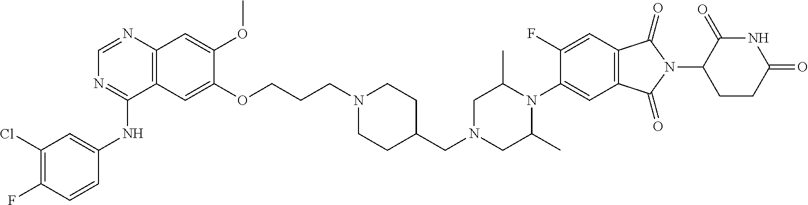 [SMILES: COc1cc2ncnc(Nc3ccc(F)c(Cl)c3)c2cc1OCCCN1CCC(CN2CC(C)N(c3cc4c(cc3F)C(=O)N(C3CCC(=O)NC3=O)C4=O)C(C)C2)CC1] | 5-(4-((1-(3-((4-((3-chloro-4- fluorophenyl)amino)-7-methoxyquinazolin-6- yl)oxy)propyl)piperidin-4-yl)methyl)-2,6- dimethylpiperazin-1-yl)-2-(2,6-dioxopiperidin- 3-yl)-6-fluoroisoindoline-1,3-dione |
| | 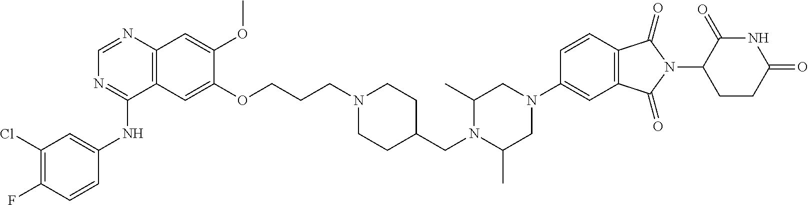 [SMILES: COc1cc2ncnc(Nc3ccc(F)c(Cl)c3)c2cc1OCCCN1CCC(CN2C(C)CN(c3ccc4c(c3)C(=O)N(C3CCC(=O)NC3=O)C4=O)CC2C)CC1] | 5-(4-((1-(3-((4-((3-chloro-4- fluorophenyl)amino)-7-methoxyquinazolin-6- yl)oxy)propyl)piperidin-4-yl)methyl)-3,5- dimethylpiperazin-1-yl)-2-(2,6-dioxopiperidin- 3-yl)isoindoline-1,3-dione |
| | 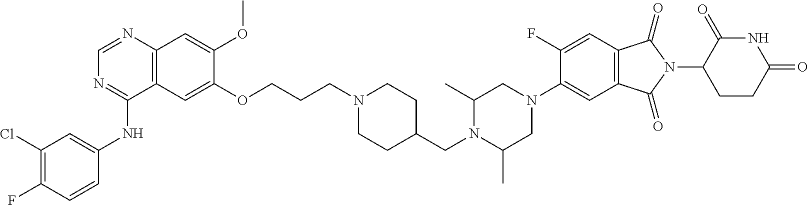 [SMILES: COc1cc2ncnc(Nc3ccc(F)c(Cl)c3)c2cc1OCCCN1CCC(CN2C(C)CN(c3cc4c(cc3F)C(=O)N(C3CCC(=O)NC3=O)C4=O)CC2C)CC1] | 5-(4-((1-(3-((4-((3-chloro-4- fluorophenyl)amino)-7-methoxyquinazolin-6- yl)oxy)propyl)piperidin-4-yl)methyl)-3,5- dimethylpiperazin-1-yl)-2-(2,6-dioxopiperidin- 3-yl)-6-fluoroisoindoline-1,3-dione |
| | 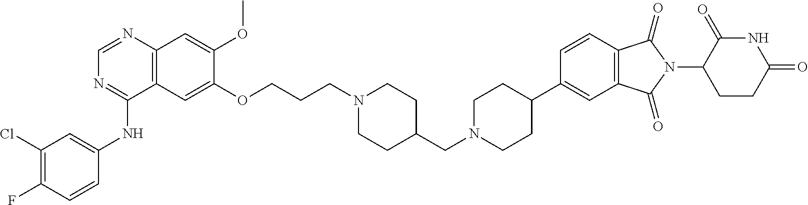 [SMILES: COc1cc2ncnc(Nc3ccc(F)c(Cl)c3)c2cc1OCCCN1CCC(CN2CCC(c3ccc4c(c3)C(=O)N(C3CCC(=O)NC3=O)C4=O)CC2)CC1] | 5-(1-((1-(3-((4-((3-chloro-4- fluorophenyl)amino)-7-methoxyquinazolin-6- yl)oxy)propyl)piperidin-4-yl)methyl)piperidin- 4-yl)-2-(2,6-dioxopiperidin-3-yl)isoindoline- 1,3-dione |
| | 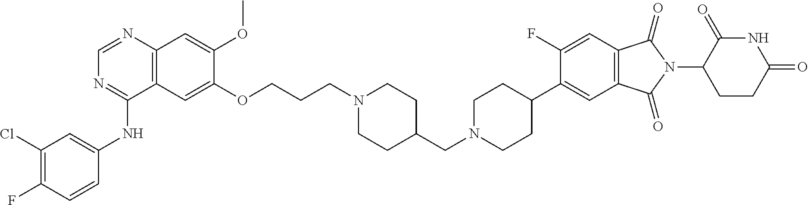 [SMILES: COc1cc2ncnc(Nc3ccc(F)c(Cl)c3)c2cc1OCCCN1CCC(CN2CCC(c3cc4c(cc3F)C(=O)N(C3CCC(=O)NC3=O)C4=O)CC2)CC1] | 5-(1-((1-(3-((4-((3-chloro-4- fluorophenyl)amino)-7-methoxyquinazolin-6- yl)oxy)propyl)piperidin-4-yl)methyl)piperidin- 4-yl)-2-(2,6-dioxopiperidin-3-yl)-6- fluoroisoindoline-1,3-dione |
| | 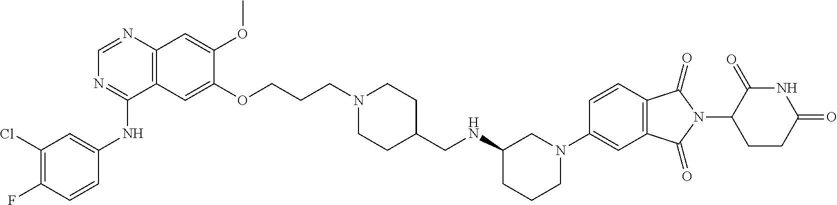 [SMILES: COc1cc2ncnc(Nc3ccc(F)c(Cl)c3)c2cc1OCCCN1CCC(CN[C@@H]2CCCN(c3ccc4c(c3)C(=O)N(C3CCC(=O)NC3=O)C4=O)C2)CC1] | 5-((R)-3-(((1-(3-((4-((3-chloro-4- fluorophenyl)amino)-7-methoxyquinazolin-6- yl)oxy)propyl)piperidin-4- yl)methyl)amino)piperidin-1-yl)-2-(2,6- dioxopiperidin-3-yl)isoindoline-1,3-dione |
| | 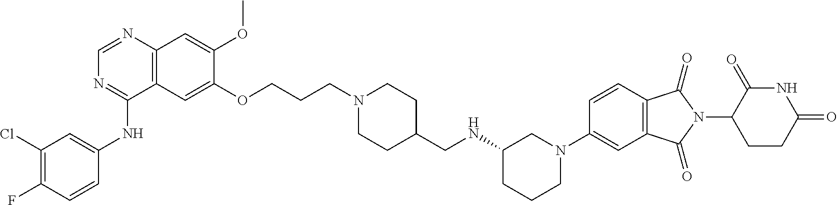 [SMILES: COc1cc2ncnc(Nc3ccc(F)c(Cl)c3)c2cc1OCCCN1CCC(CN[C@H]2CCCN(c3ccc4c(c3)C(=O)N(C3CCC(=O)NC3=O)C4=O)C2)CC1] | 5-((S)-3-(((1-(3-((4-((3-chloro-4- fluorophenyl)amino)-7-methoxyquinazolin-6- yl)oxy)propyl)piperidin-4- yl)methyl)amino)piperidin-1-yl)-2-(2,6- dioxopiperidin-3-yl)isoindoline-1,3-dione |
| | 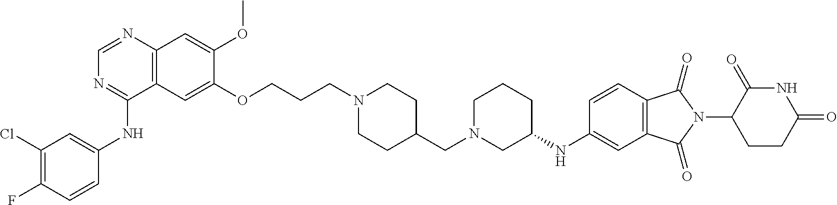 [SMILES: COc1cc2ncnc(Nc3ccc(F)c(Cl)c3)c2cc1OCCCN1CCC(CN2CCC[C@H](Nc3ccc4c(c3)C(=O)N(C3CCC(=O)NC3=O)C4=O)C2)CC1] | 5-(((S)-1-((1-(3-((4-((3-chloro-4- fluorophenyl)amino)-7-methoxyquinazolin-6- yl)oxy)propyl)piperidin-4-yl)methyl)piperidin- 3-yl)amino)-2-(2,6-dioxopiperidin-3- yl)isoindoline-1,3-dione |
| | 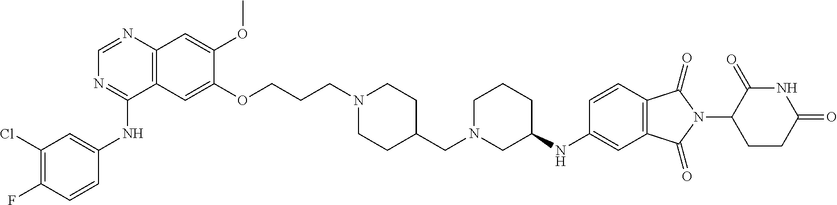 [SMILES: COc1cc2ncnc(Nc3ccc(F)c(Cl)c3)c2cc1OCCCN1CCC(CN2CCC[C@@H](Nc3ccc4c(c3)C(=O)N(C3CCC(=O)NC3=O)C4=O)C2)CC1] | 5-(((R)-1-((1-(3-((4-((3-chloro-4- fluorophenyl)amino)-7-methoxyquinazolin-6- yl)oxy)propyl)piperidin-4-yl)methyl)piperidin- 3-yl)amino)-2-(2,6-dioxopiperidin-3- yl)isoindoline-1,3-dione |
| | 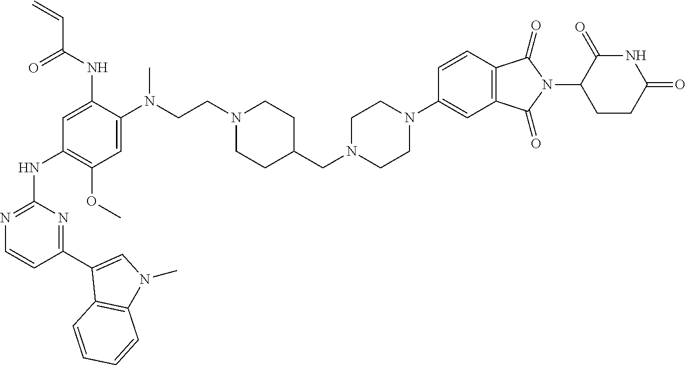 [SMILES: C=CC(=O)Nc1cc(Nc2nccc(-c3cn(C)c4ccccc34)n2)c(OC)cc1N(C)CCN1CCC(CN2CCN(c3ccc4c(c3)C(=O)N(C3CCC(=O)NC3=O)C4=O)CC2)CC1] | N-(2-((2-(4-((4-(2-(2,6-dioxopiperidin-3-yl)- 1,3-dioxoisoindolin-5-yl)piperazin-1 - yl)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 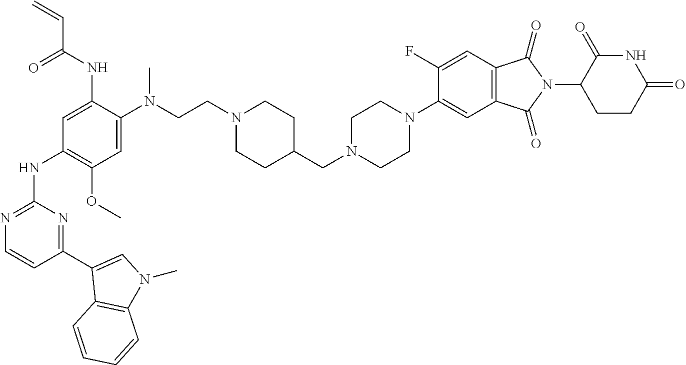 [SMILES: C=CC(=O)Nc1cc(Nc2nccc(-c3cn(C)c4ccccc34)n2)c(OC)cc1N(C)CCN1CCC(CN2CCN(c3cc4c(cc3F)C(=O)N(C3CCC(=O)NC3=O)C4=O)CC2)CC1] | N-(2-((2-(4-((4-(2-(2,6-dioxopiperidin-3-yl)-6- fluoro-1,3-dioxoisoindolin-5-yl)piperazin-1- yl)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 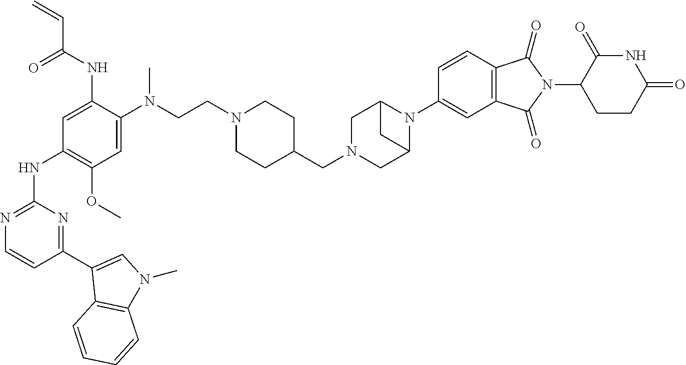 [SMILES: C=CC(=O)Nc1cc(Nc2nccc(-c3cn(C)c4ccccc34)n2)c(OC)cc1N(C)CCN1CCC(CN2CC3CC(C2)N3c2ccc3c(c2)C(=O)N(C2CCC(=O)NC2=O)C3=O)CC1] | N-(2-((2-(4-((6-(2-(2,6-dioxopiperidin-3-yl)- 1,3-dioxoisoindolin-5-yl)-3,6- diazabicyclo[3.1.1]heptan-3- yl)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 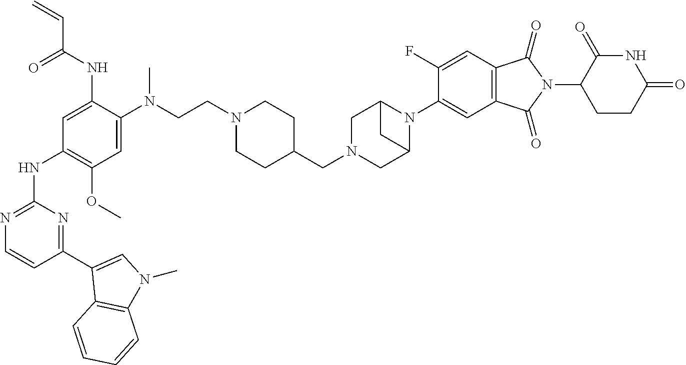 [SMILES: C=CC(=O)Nc1cc(Nc2nccc(-c3cn(C)c4ccccc34)n2)c(OC)cc1N(C)CCN1CCC(CN2CC3CC(C2)N3c2cc3c(cc2F)C(=O)N(C2CCC(=O)NC2=O)C3=O)CC1] | N-(2-((2-(4-((6-(2-(2,6-dioxopiperidin-3-yl)-6- fluoro-1,3-dioxoisoindolin-5-yl)-3,6- diazabicyclo[3.1.1]heptan-3- yl)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 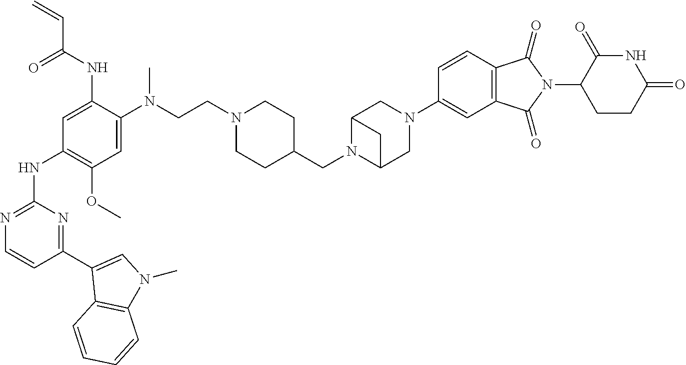 [SMILES: C=CC(=O)Nc1cc(Nc2nccc(-c3cn(C)c4ccccc34)n2)c(OC)cc1N(C)CCN1CCC(CN2C3CC2CN(c2ccc4c(c2)C(=O)N(C2CCC(=O)NC2=O)C4=O)C3)CC1] | N-(2-((2-(4-((3-(2-(2,6-dioxopiperidin-3-yl)- 1,3-dioxoisoindolin-5-yl)-3,6- diazabicyclo[3.1.1]heptan-6- yl)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 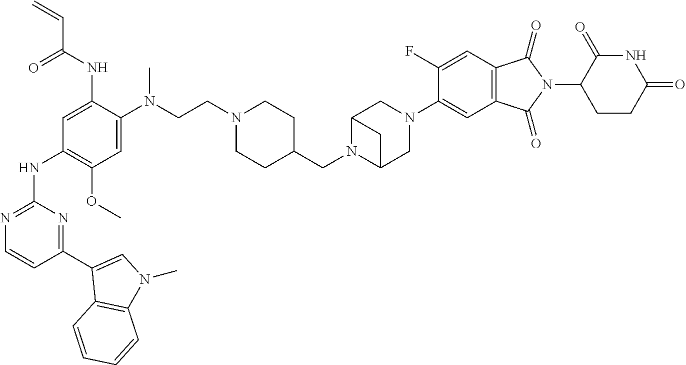 [SMILES: C=CC(=O)Nc1cc(Nc2nccc(-c3cn(C)c4ccccc34)n2)c(OC)cc1N(C)CCN1CCC(CN2C3CC2CN(c2cc4c(cc2F)C(=O)N(C2CCC(=O)NC2=O)C4=O)C3)CC1] | N-(2-((2-(4-((3-(2-(2,6-dioxopiperidin-3-yl)-6- fluoro-1,3-dioxoisoindolin-5-yl)-3,6- diazabicyclo[3.1.1]heptan-6- yl)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 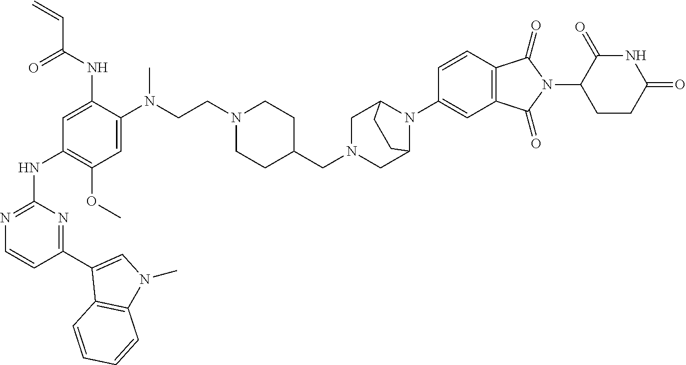 [SMILES: C=CC(=O)Nc1cc(Nc2nccc(-c3cn(C)c4ccccc34)n2)c(OC)cc1N(C)CCN1CCC(CN2CC3CCC(C2)N3c2ccc3c(c2)C(=O)N(C2CCC(=O)NC2=O)C3=O)CC1] | N-(2-((2-(4-((8-(2-(2,6-dioxopiperidin-3-yl)- 1,3-dioxoisoindolin-5-yl)-3,8- diazabicyclo[3.2.1]octan-3-yl)methyl)piperidin- 1-yl)ethyl)(methyl)amino)-4-methoxy-5-((4-(1- methyl-1H-indol-3-yl)pyrimidin-2- yl)amino)phenyl)acrylamide |
| | 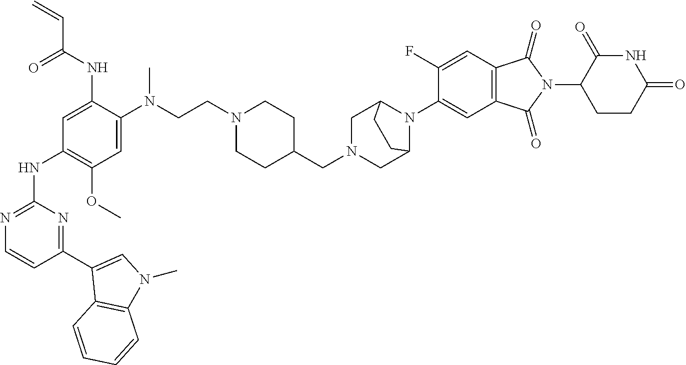 [SMILES: C=CC(=O)Nc1cc(Nc2nccc(-c3cn(C)c4ccccc34)n2)c(OC)cc1N(C)CCN1CCC(CN2CC3CCC(C2)N3c2cc3c(cc2F)C(=O)N(C2CCC(=O)NC2=O)C3=O)CC1] | N-(2-((2-(4-((8-(2-(2,6-dioxopiperidin-3-yl)-6- fluoro-1,3-dioxoisoindolin-5-yl)-3,8- diazabicyclo[3.2.1]octan-3-yl)methyl)piperidin- 1-yl)ethyl)(methyl)amino)-4-methoxy-5-((4-(1- methyl-1H-indol-3-yl)pyrimidin-2- yl)amino)phenyl)acrylamide |
| | 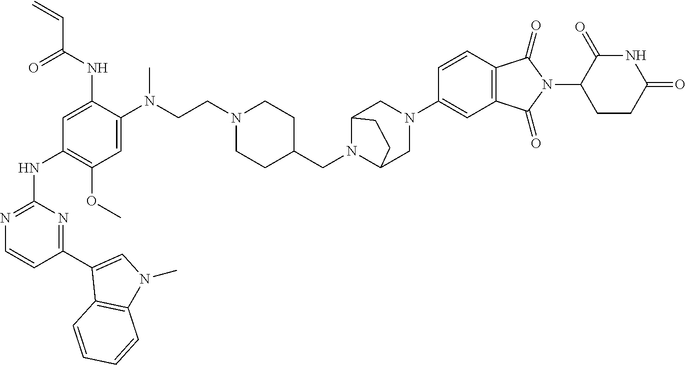 [SMILES: C=CC(=O)Nc1cc(Nc2nccc(-c3cn(C)c4ccccc34)n2)c(OC)cc1N(C)CCN1CCC(CN2C3CCC2CN(c2ccc4c(c2)C(=O)N(C2CCC(=O)NC2=O)C4=O)C3)CC1] | N-(2-((2-(4-((3-(2-(2,6-dioxopiperidin-3-yl)- 1,3-dioxoisoindolin-5-yl)-3,8- diazabicyclo[3.2.1]octan-8-yl)methyl)piperidin- 1-yl)ethyl)(methyl)amino)-4-methoxy-5-((4-(1- methyl-1H-indol-3-yl)pyrimidin-2- yl)amino)phenyl)acrylamide |
| | 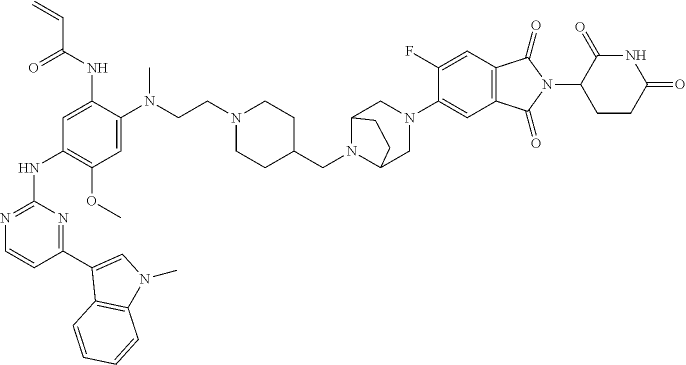 [SMILES: C=CC(=O)Nc1cc(Nc2nccc(-c3cn(C)c4ccccc34)n2)c(OC)cc1N(C)CCN1CCC(CN2C3CCC2CN(c2cc4c(cc2F)C(=O)N(C2CCC(=O)NC2=O)C4=O)C3)CC1] | N-(2-((2-(4-((3-(2-(2,6-dioxopiperidin-3-yl)-6- fluoro-1,3-dioxoisoindolin-5-yl)-3,8- diazabicyclo[3.2.1]octan-8-yl)methyl)piperidin- 1-yl)ethyl)(methyl)amino)-4-methoxy-5-((4-(1- methyl-1H-indol-3-yl)pyrimidin-2- yl)amino)phenyl)acrylamide |
| | 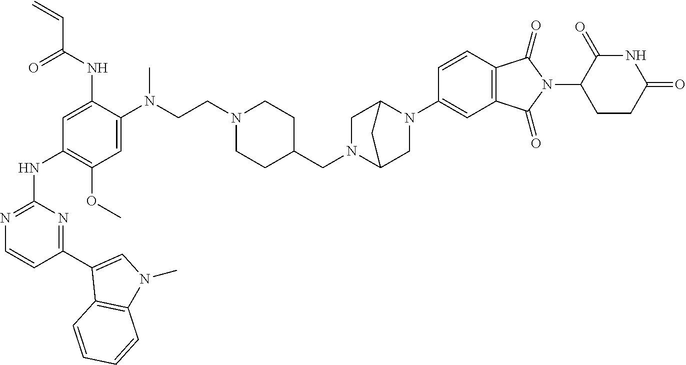 [SMILES: C=CC(=O)Nc1cc(Nc2nccc(-c3cn(C)c4ccccc34)n2)c(OC)cc1N(C)CCN1CCC(CN2CC3CC2CN3c2ccc3c(c2)C(=O)N(C2CCC(=O)NC2=O)C3=O)CC1] | N-(2-((2-(4-((5-(2-(2,6-dioxopiperidin-3-yl)- 1,3-dioxoisoindolin-5-yl)-2,5- diazabicyclo[2.2.1]heptan-2- yl)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 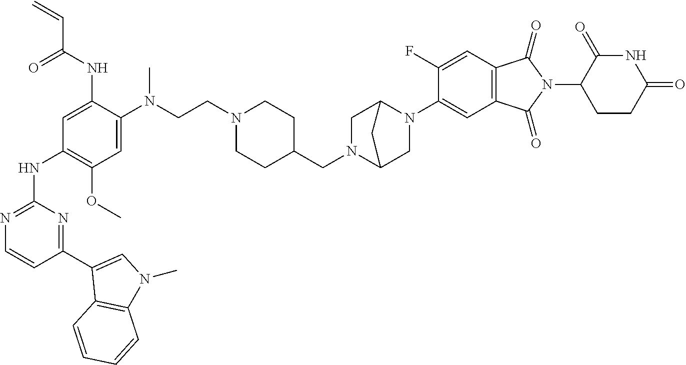 [SMILES: C=CC(=O)Nc1cc(Nc2nccc(-c3cn(C)c4ccccc34)n2)c(OC)cc1N(C)CCN1CCC(CN2CC3CC2CN3c2cc3c(cc2F)C(=O)N(C2CCC(=O)NC2=O)C3=O)CC1] | N-(2-((2-(4-((5-(2-(2,6-dioxopiperidin-3-yl)-6- fluoro-1,3-dioxoisoindolin-5-yl)-2,5- diazabicyclo[2.2.1]heptan-2- yl)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 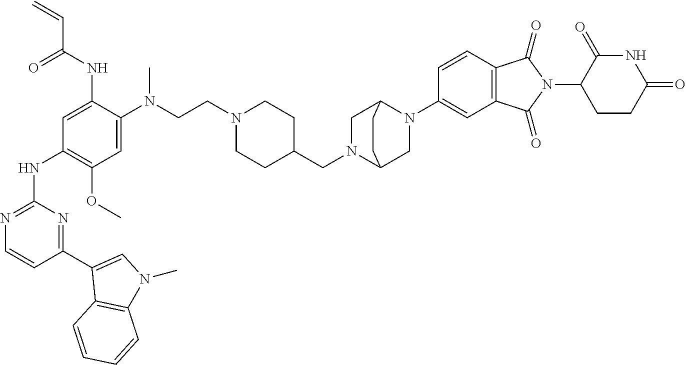 [SMILES: C=CC(=O)Nc1cc(Nc2nccc(-c3cn(C)c4ccccc34)n2)c(OC)cc1N(C)CCN1CCC(CN2CC3CCC2CN3c2ccc3c(c2)C(=O)N(C2CCC(=O)NC2=O)C3=O)CC1] | N-(2-((2-(4-((5-(2-(2,6-dioxopiperidin-3-yl)- 1,3-dioxoisoindolin-5-yl)-2,5- diazabicyclo[2.2.2]octan-2-yl)methyl)piperidin- 1-yl)ethyl)(methyl)amino)-4-methoxy-5-((4-(1- methyl-1H-indol-3-yl)pyrimidin-2- yl)amino)phenyl)acrylamide |
| | 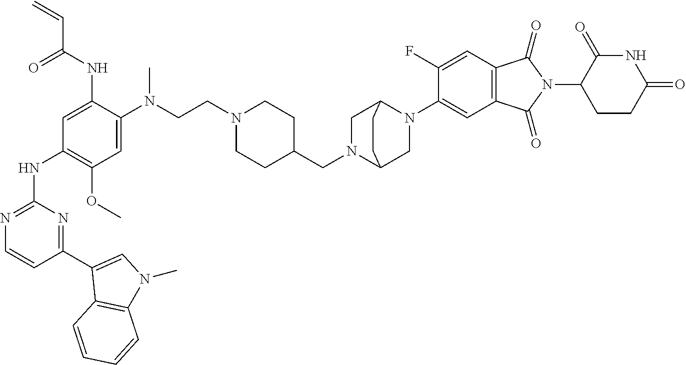 [SMILES: C=CC(=O)Nc1cc(Nc2nccc(-c3cn(C)c4ccccc34)n2)c(OC)cc1N(C)CCN1CCC(CN2CC3CCC2CN3c2cc3c(cc2F)C(=O)N(C2CCC(=O)NC2=O)C3=O)CC1] | N-(2-((2-(4-((5-(2-(2,6-dioxopiperidin-3-yl)-6- fluoro-1,3-dioxoisoindolin-5-yl)-2,5- diazabicyclo[2.2.2]octan-2-yl)methyl)piperidin- 1-yl)ethyl)(methyl)amino)-4-methoxy-5-((4-(1- methyl-1H-indol-3-yl)pyrimidin-2- yl)amino)phenyl)acrylamide |
| | 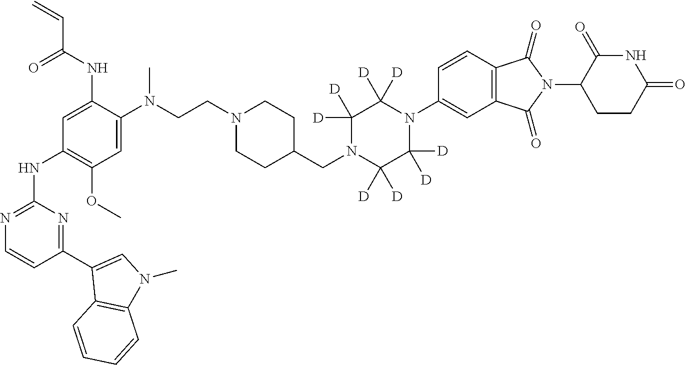 [SMILES: [2H]C1([2H])N(CC2CCN(CCN(C)c3cc(OC)c(Nc4nccc(-c5cn(C)c6ccccc56)n4)cc3NC(=O)C=C)CC2)C([2H])([2H])C([2H])([2H])N(c2ccc3c(c2)C(=O)N(C2CCC(=O)NC2=O)C3=O)C1([2H])[2H]] | N-(2-((2-(4-((4-(2-(2,6-dioxopiperidin-3-yl)- 1,3-dioxoisoindolin-5-yl)piperazin-1-yl- 2,2,3,3,5,5,6,6-d₈)methyl)piperidin-1- yl)ethyl)(methyl)amino)-4-methoxy-5-((4-(1- methyl-1H-indol-3-yl)pyrimidin-2- yl)amino)phenyl)acrylamide |
| | 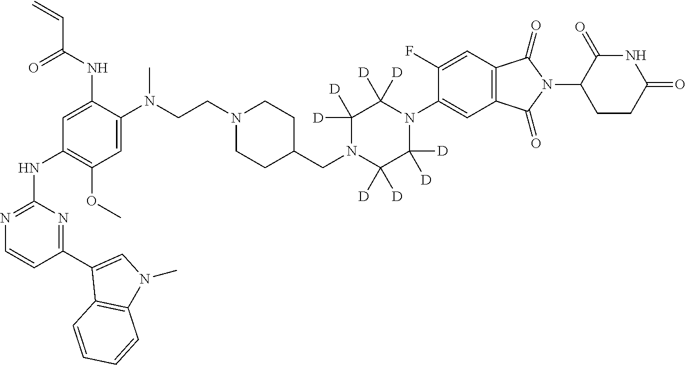 [SMILES: [2H]C1([2H])N(CC2CCN(CCN(C)c3cc(OC)c(Nc4nccc(-c5cn(C)c6ccccc56)n4)cc3NC(=O)C=C)CC2)C([2H])([2H])C([2H])([2H])N(c2cc3c(cc2F)C(=O)N(C2CCC(=O)NC2=O)C3=O)C1([2H])[2H]] | N-(2-((2-(4-((4-(2-(2,6-dioxopiperidin-3-yl)-6- fluoro-1,3-dioxoisoindolin-5-yl)piperazin-1-yl- 2,2,3,3,5,5,6,6-d₈)methyl)piperidin-1- yl)ethyl)(methyl)amino)-4-methoxy-5-((4-(1- methyl-1H-indol-3-yl)pyrimidin-2- yl)amino)phenyl)acrylamide |
| | 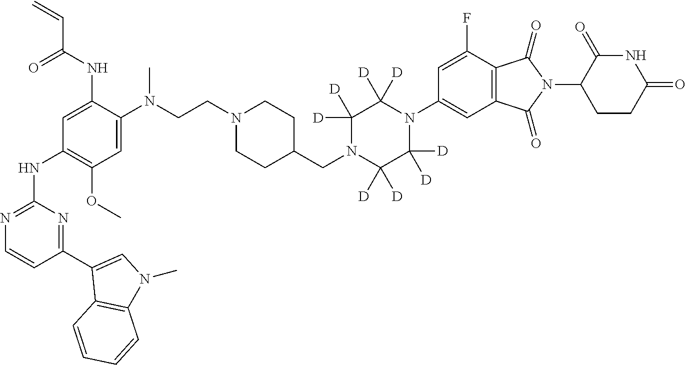 [SMILES: [2H]C1([2H])N(CC2CCN(CCN(C)c3cc(OC)c(Nc4nccc(-c5cn(C)c6ccccc56)n4)cc3NC(=O)C=C)CC2)C([2H])([2H])C([2H])([2H])N(c2cc(F)c3c(c2)C(=O)N(C2CCC(=O)NC2=O)C3=O)C1([2H])[2H]] | N-(2-((2-(4-((4-(2-(2,6-dioxopiperidin-3-yl)-7- fluoro-1,3-dioxoisoindolin-5-yl)piperazin-1-yl- 2,2,3,3,5,5,6,6-d₈)methyl)piperidin-1- yl)ethyl)(methyl)amino)-4-methoxy-5-((4-(1- methyl-1H-indol-3-yl)pyrimidin-2- yl)amino)phenyl)acrylamide |
| | 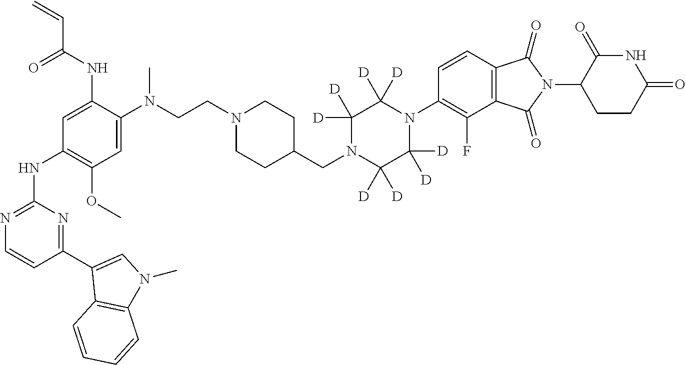 [SMILES: [2H]C1([2H])N(CC2CCN(CCN(C)c3cc(OC)c(Nc4nccc(-c5cn(C)c6ccccc56)n4)cc3NC(=O)C=C)CC2)C([2H])([2H])C([2H])([2H])N(c2ccc3c(c2F)C(=O)N(C2CCC(=O)NC2=O)C3=O)C1([2H])[2H]] | N-(2-((2-(4-((4-(2-(2,6-dioxopiperidin-3-yl)-4- fluoro-1,3-dioxoisoindolin-5-yl)piperazin-1-yl- 2,2,3,3,5,5,6,6-d₈)methyl)piperidin-1- yl)ethyl)(methyl)amino)-4-methoxy-5-((4-(1- methyl-1H-indol-3-yl)pyrimidin-2- yl)amino)phenyl)acrylamide |
| | 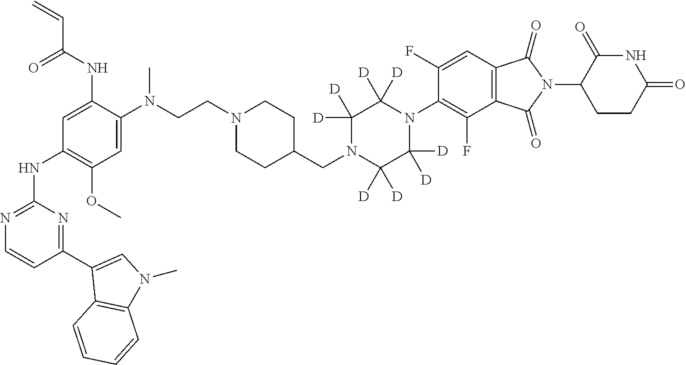 [SMILES: [2H]C1([2H])N(CC2CCN(CCN(C)c3cc(OC)c(Nc4nccc(-c5cn(C)c6ccccc56)n4)cc3NC(=O)C=C)CC2)C([2H])([2H])C([2H])([2H])N(c2c(F)cc3c(c2F)C(=O)N(C2CCC(=O)NC2=O)C3=O)C1([2H])[2H]] | N-(2-((2-(4-((4-(2-(2,6-dioxopiperidin-3-yl)- 4,6-difluoro-1,3-dioxoisoindolin-5- yl)piperazin-1-yl-2,2,3,3,5,5,6,6- d₈)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 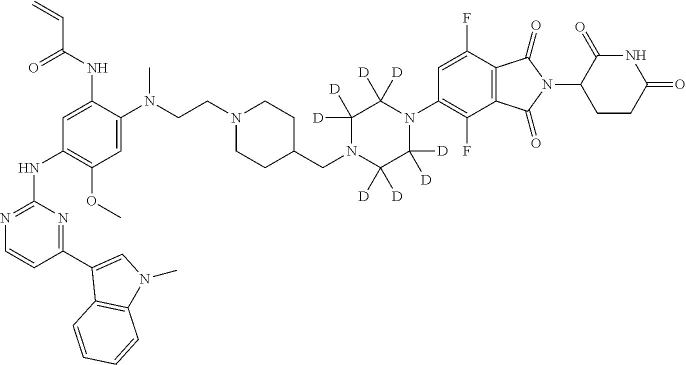 [SMILES: [2H]C1([2H])N(CC2CCN(CCN(C)c3cc(OC)c(Nc4nccc(-c5cn(C)c6ccccc56)n4)cc3NC(=O)C=C)CC2)C([2H])([2H])C([2H])([2H])N(c2cc(F)c3c(c2F)C(=O)N(C2CCC(=O)NC2=O)C3=O)C1([2H])[2H]] | N-(2-((2-(4-((4-(2-(2,6-dioxopiperidin-3-yl)- 4,7-difluoro-1,3-dioxoisoindolin-5- yl)piperazin-1-yl-2,2,3,3,5,5,6,6- d₈)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 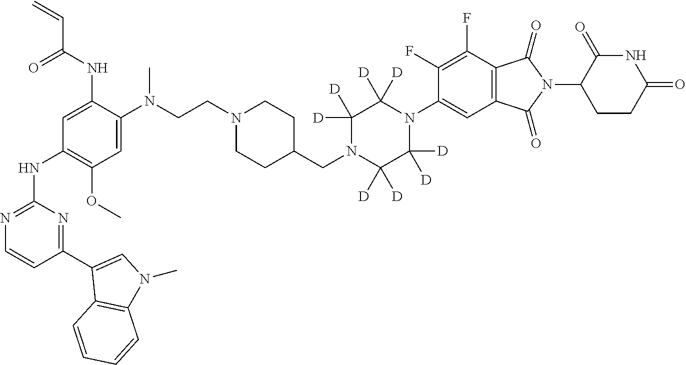 [SMILES: [2H]C1([2H])N(CC2CCN(CCN(C)c3cc(OC)c(Nc4nccc(-c5cn(C)c6ccccc56)n4)cc3NC(=O)C=C)CC2)C([2H])([2H])C([2H])([2H])N(c2cc3c(c(F)c2F)C(=O)N(C2CCC(=O)NC2=O)C3=O)C1([2H])[2H]] | N-(2-((2-(4-((4-(2-(2,6-dioxopiperidin-3-yl)- 6,7-difluoro-1,3-dioxoisoindolin-5- yl)piperazin-1-yl-2,2,3,3,5,5,6,6- d₈)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 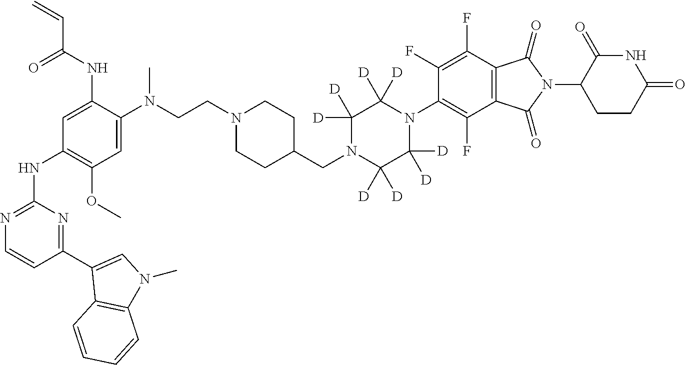 [SMILES: [2H]C1([2H])N(CC2CCN(CCN(C)c3cc(OC)c(Nc4nccc(-c5cn(C)c6ccccc56)n4)cc3NC(=O)C=C)CC2)C([2H])([2H])C([2H])([2H])N(c2c(F)c(F)c3c(c2F)C(=O)N(C2CCC(=O)NC2=O)C3=O)C1([2H])[2H]] | N-(2-((2-(4-((4-(2-(2,6-dioxopiperidin-3-yl)- 4,6,7-trifluoro-1,3-dioxoisoindolin-5- yl)piperazin-1-yl-2,2,3,3,5,5,6,6- d₈)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 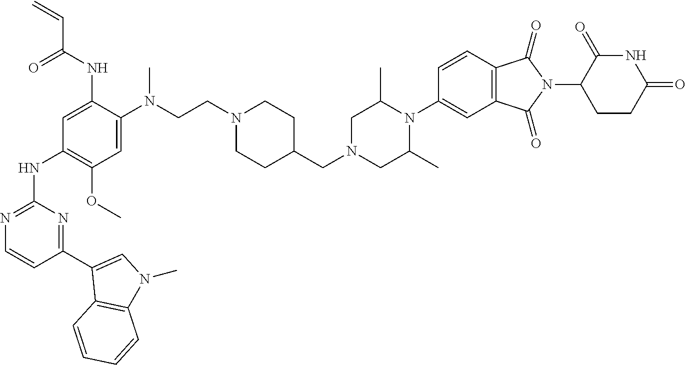 [SMILES: C=CC(=O)Nc1cc(Nc2nccc(-c3cn(C)c4ccccc34)n2)c(OC)cc1N(C)CCN1CCC(CN2CC(C)N(c3ccc4c(c3)C(=O)N(C3CCC(=O)NC3=O)C4=O)C(C)C2)CC1] | N-(2-((2-(4-((4-(2-(2,6-dioxopiperidin-3-yl)- 1,3-dioxoisoindolin-5-yl)-3,5- dimethylpiperazin-1-yl)methyl)piperidin-1- yl)ethyl)(methyl)amino)-4-methoxy-5-((4-(1- methyl-1H-indol-3-yl)pyrimidin-2- yl)amino)phenyl)acrylamide |
| | 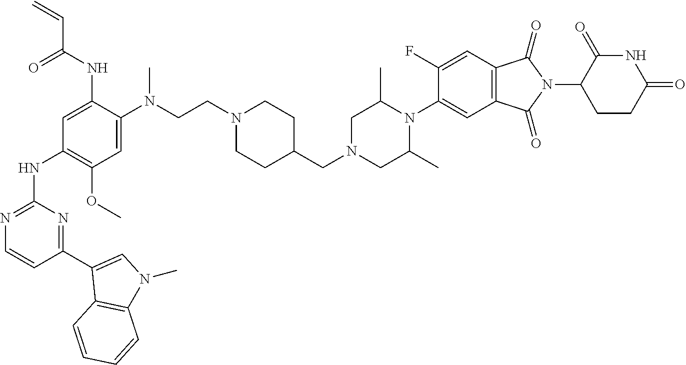 [SMILES: C=CC(=O)Nc1cc(Nc2nccc(-c3cn(C)c4ccccc34)n2)c(OC)cc1N(C)CCN1CCC(CN2CC(C)N(c3cc4c(cc3F)C(=O)N(C3CCC(=O)NC3=O)C4=O)C(C)C2)CC1] | N-(2-((2-(4-((4-(2-(2,6-dioxopiperidin-3-yl)-6- fluoro-1,3-dioxoisoindolin-5-yl)-3,5- dimethylpiperazin-1-yl)methyl)piperidin-1- yl)ethyl)(methyl)amino)-4-methoxy-5-((4-(1- methyl-1H-indol-3-yl)pyrimidin-2- yl)amino)phenyl)acrylamide |
| | 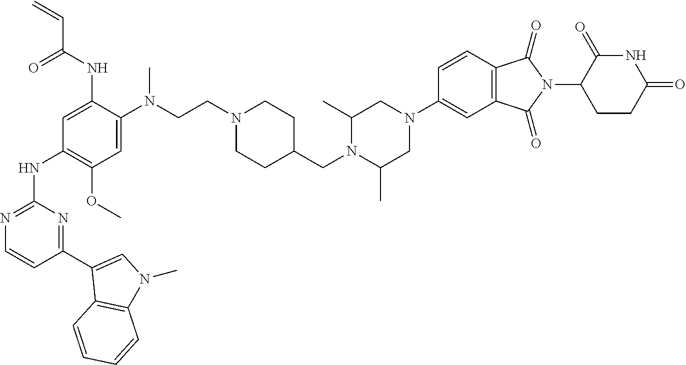 [SMILES: C=CC(=O)Nc1cc(Nc2nccc(-c3cn(C)c4ccccc34)n2)c(OC)cc1N(C)CCN1CCC(CN2C(C)CN(c3ccc4c(c3)C(=O)N(C3CCC(=O)NC3=O)C4=O)CC2C)CC1] | N-(2-((2-(4-((4-(2-(2,6-dioxopiperidin-3-yl)- 1,3-dioxoisoindolin-5-yl)-2,6- dimethylpiperazin-1-yl)methyl)piperidin-1- yl)ethyl)(methyl)amino)-4-methoxy-5-((4-(1- methyl-1H-indol-3-yl)pyrimidin-2- yl)amino)phenyl)acrylamide |
| | 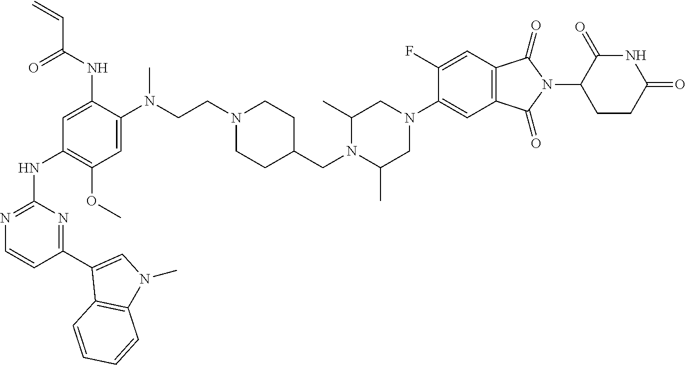 [SMILES: C=CC(=O)Nc1cc(Nc2nccc(-c3cn(C)c4ccccc34)n2)c(OC)cc1N(C)CCN1CCC(CN2C(C)CN(c3cc4c(cc3F)C(=O)N(C3CCC(=O)NC3=O)C4=O)CC2C)CC1] | N-(2-((2-(4-((4-(2-(2,6-dioxopiperidin-3-yl)-6- fluoro-1,3-dioxoisoindolin-5-yl)-2,6- dimethylpiperazin-1-yl)methyl)piperidin-1- yl)ethyl)(methyl)amino)-4-methoxy-5-((4-(1- methyl-1H-indol-3-yl)pyrimidin-2- yl)amino)phenyl)acrylamide |
| | 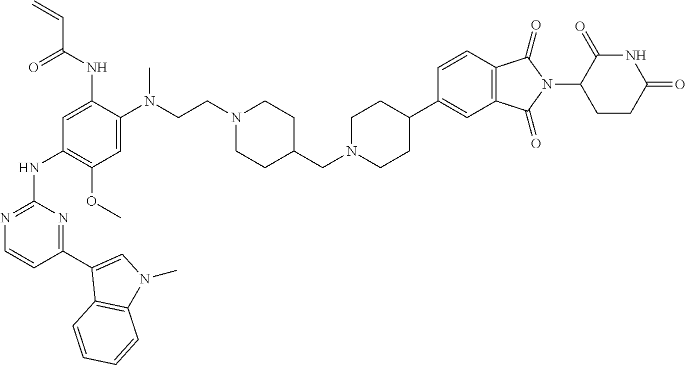 [SMILES: C=CC(=O)Nc1cc(Nc2nccc(-c3cn(C)c4ccccc34)n2)c(OC)cc1N(C)CCN1CCC(CN2CCC(c3ccc4c(c3)C(=O)N(C3CCC(=O)NC3=O)C4=O)CC2)CC1] | N-(2-((2-(4-((4-(2-(2,6-dioxopiperidin-3-yl)- 1,3-dioxoisoindolin-5-yl)piperidin-1- yl)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 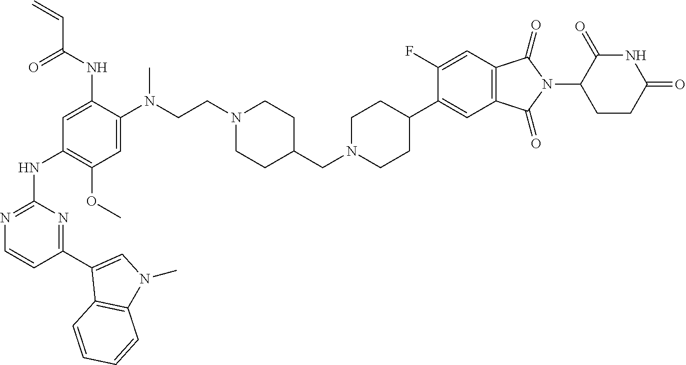 [SMILES: C=CC(=O)Nc1cc(Nc2nccc(-c3cn(C)c4ccccc34)n2)c(OC)cc1N(C)CCN1CCC(CN2CCC(c3cc4c(cc3F)C(=O)N(C3CCC(=O)NC3=O)C4=O)CC2)CC1] | N-(2-((2-(4-((4-(2-(2,6-dioxopiperidin-3-yl)-6- fluoro-1,3-dioxoisoindolin-5-yl)piperidin-1- yl)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 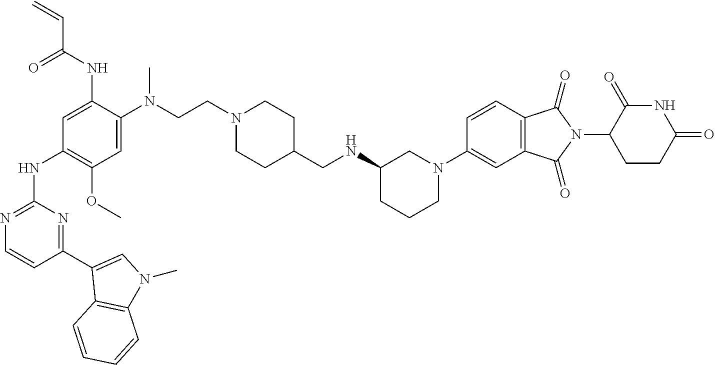 [SMILES: C=CC(=O)Nc1cc(Nc2nccc(-c3cn(C)c4ccccc34)n2)c(OC)cc1N(C)CCN1CCC(CN[C@@H]2CCCN(c3ccc4c(c3)C(=O)N(C3CCC(=O)NC3=O)C4=O)C2)CC1] | N-(2-((2-(4-((((3R)-1-(2-(2,6-dioxopiperidin-3- yl)-1,3-dioxoisoindolin-5-yl)piperidin-3- yl)amino)methyl)piperidin-1- yl)ethyl)(methyl)amino)-4-methoxy-5-((4-(1- methyl-1H-indol-3-yl)pyrimidin-2- yl)amino)phenyl)acrylamide |
| | 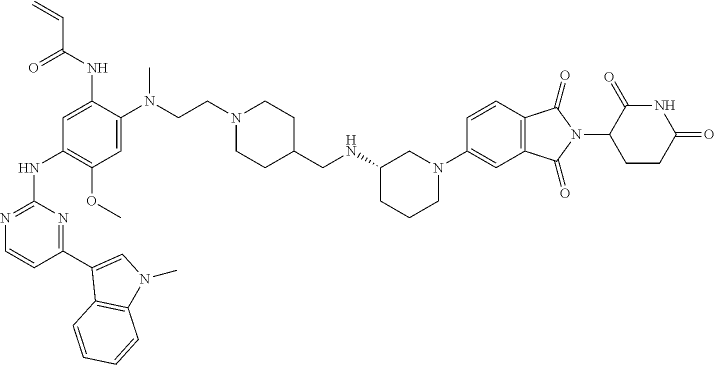 [SMILES: C=CC(=O)Nc1cc(Nc2nccc(-c3cn(C)c4ccccc34)n2)c(OC)cc1N(C)CCN1CCC(CN[C@H]2CCCN(c3ccc4c(c3)C(=O)N(C3CCC(=O)NC3=O)C4=O)C2)CC1] | N-(2-((2-(4-((((3S)-1-(2-(2,6-dioxopiperidin-3- yl)-1,3-dioxoisoindolin-5-yl)piperidin-3- yl)amino)methyl)piperidin-1- yl)ethyl)(methyl)amino)-4-methoxy-5-((4-(1- methyl-1H-indol-3-yl)pyrimidin-2- yl)amino)phenyl)acrylamide |
| | 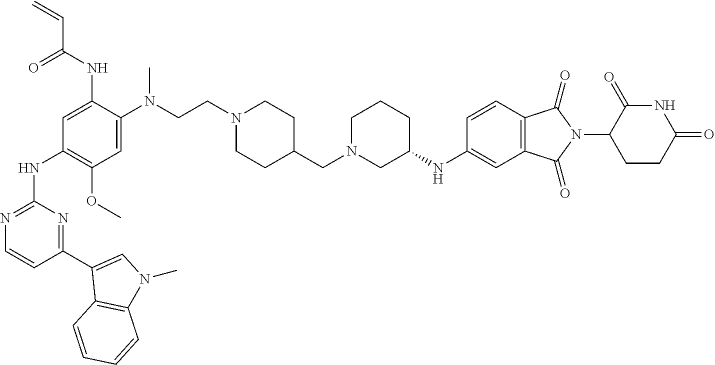 [SMILES: C=CC(=O)Nc1cc(Nc2nccc(-c3cn(C)c4ccccc34)n2)c(OC)cc1N(C)CCN1CCC(CN2CCC[C@H](Nc3ccc4c(c3)C(=O)N(C3CCC(=O)NC3=O)C4=O)C2)CC1] | N-(2-((2-(4-(((3S)-3-((2-(2,6-dioxopiperidin-3- yl)-1,3-dioxoisoindolin-5-yl)amino)piperidin-1- yl)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 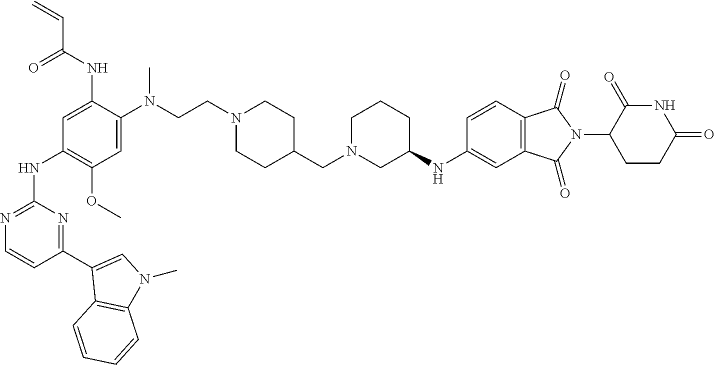 [SMILES: C=CC(=O)Nc1cc(Nc2nccc(-c3cn(C)c4ccccc34)n2)c(OC)cc1N(C)CCN1CCC(CN2CCC[C@@H](Nc3ccc4c(c3)C(=O)N(C3CCC(=O)NC3=O)C4=O)C2)CC1] | N-(2-((2-(4-(((3R)-3-((2-(2,6-dioxopiperidin-3- yl)-1,3-dioxoisoindolin-5-yl)amino)piperidin-1- yl)methyl)piperidin-1-yl)ethyl)(methyl)amino)- 4-methoxy-5-((4-(1-methyl-1H-indol-3- yl)pyrimidin-2-yl)amino)phenyl)acrylamide |
| | 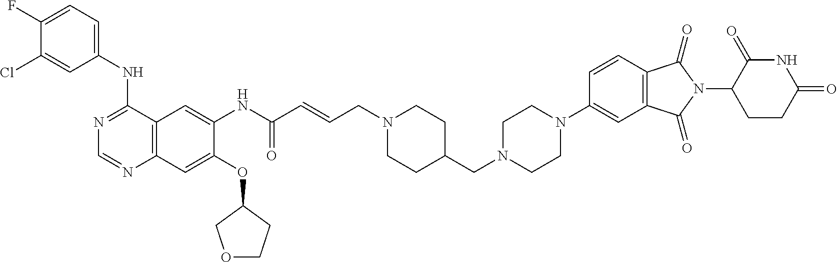 [SMILES: O=C(/C=C/CN1CCC(CN2CCN(c3ccc4c(c3)C(=O)N(C3CCC(=O)NC3=O)C4=O)CC2)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-1,3- dioxoisoindolin-5-yl)piperazin-1- yl)methyl)piperidin-1-yl)but-2-enamide |
| | 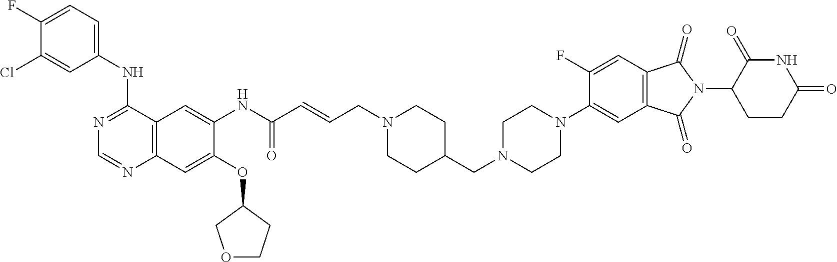 [SMILES: O=C(/C=C/CN1CCC(CN2CCN(c3cc4c(cc3F)C(=O)N(C3CCC(=O)NC3=O)C4=O)CC2)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-6- fluoro-1,3-dioxoisoindolin-5-yl)piperazin-1- yl)methyl)piperidin-1-yl)but-2-enamide |
| | 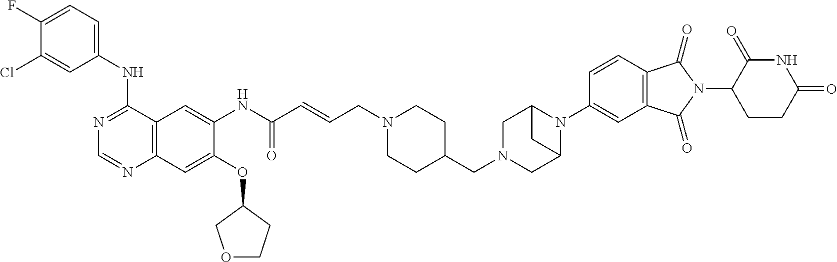 [SMILES: O=C(/C=C/CN1CCC(CN2CC3CC(C2)N3c2ccc3c(c2)C(=O)N(C2CCC(=O)NC2=O)C3=O)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((6-(2-(2,6-dioxopiperidin-3-yl)-1,3- dioxoisoindolin-5-yl)-3,6- diazabicyclo[3.1.1]heptan-3- yl)methyl)piperidin-1-yl)but-2-enamide |
| | 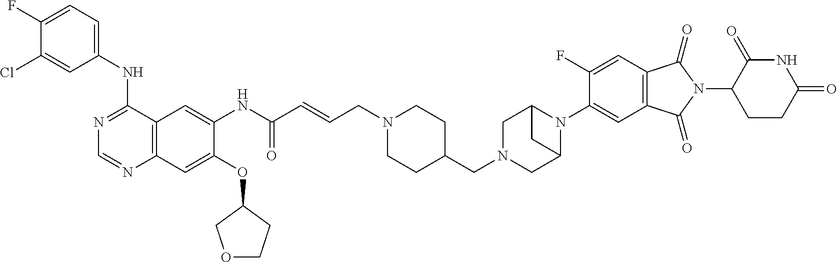 [SMILES: O=C(/C=C/CN1CCC(CN2CC3CC(C2)N3c2cc3c(cc2F)C(=O)N(C2CCC(=O)NC2=O)C3=O)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((6-(2-(2,6-dioxopiperidin-3-yl)-6- fluoro-1,3-dioxoisoindolin-5-yl)-3,6- diazabicyclo[3.1.1]heptan-3- yl)methyl)piperidin-1-yl)but-2-enamide |
| | 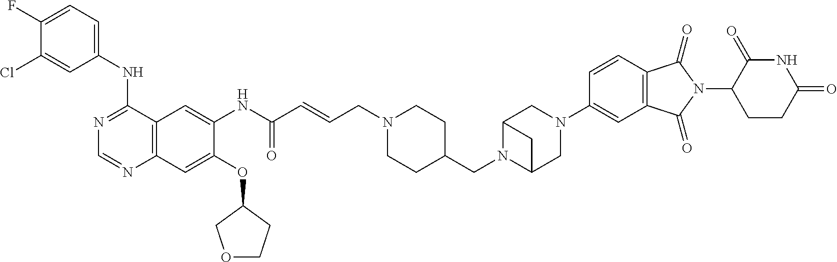 [SMILES: O=C(/C=C/CN1CCC(CN2C3CC2CN(c2ccc4c(c2)C(=O)N(C2CCC(=O)NC2=O)C4=O)C3)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((3-(2-(2,6-dioxopiperidin-3-yl)-1,3- dioxoisoindolin-5-yl)-3,6- diazabicyclo[3.1.1]heptan-6- yl)methyl)piperidin-1-yl)but-2-enamide |
| | 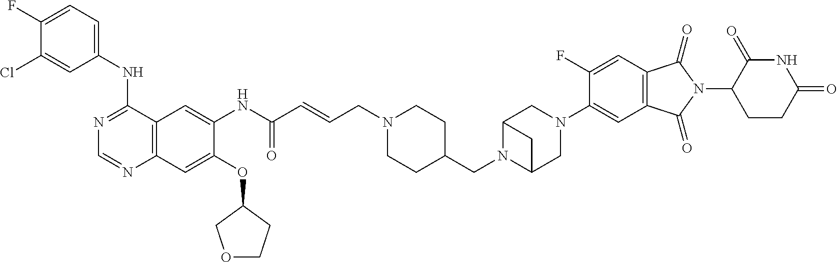 [SMILES: O=C(/C=C/CN1CCC(CN2C3CC2CN(c2cc4c(cc2F)C(=O)N(C2CCC(=O)NC2=O)C4=O)C3)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((3-(2-(2,6-dioxopiperidin-3-yl)-6- fluoro-1,3-dioxoisoindolin-5-yl)-3,6- diazabicyclo[3.1.1]heptan-6- yl)methyl)piperidin-1-yl)but-2-enamide |
| | 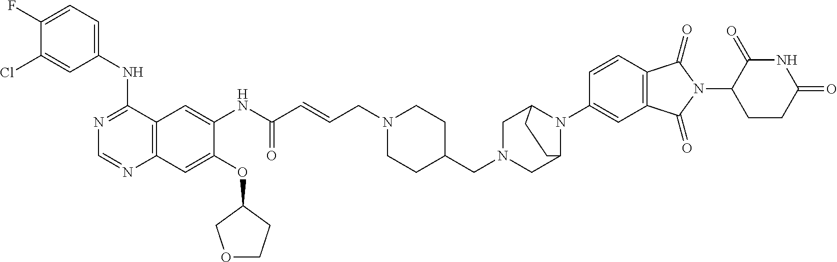 [SMILES: O=C(/C=C/CN1CCC(CN2CC3CCC(C2)N3c2ccc3c(c2)C(=O)N(C2CCC(=O)NC2=O)C3=O)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((8-(2-(2,6-dioxopiperidin-3-yl)-1,3- dioxoisoindolin-5-yl)-3,8- diazabicyclo[3.2.1]octan-3-yl)methyl)piperidin- 1-yl)but-2-enamide |
| | 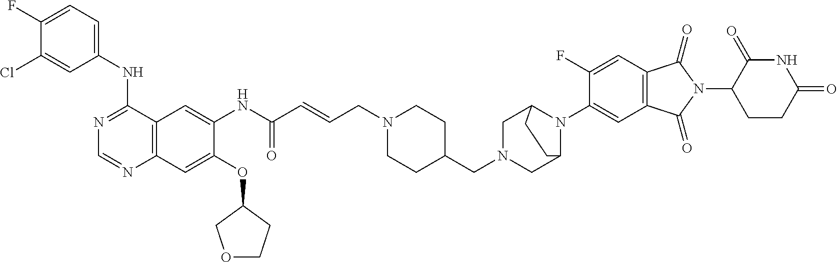 [SMILES: O=C(/C=C/CN1CCC(CN2CC3CCC(C2)N3c2cc3c(cc2F)C(=O)N(C2CCC(=O)NC2=O)C3=O)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((8-(2-(2,6-dioxopiperidin-3-yl)-6- fluoro-1,3-dioxoisoindolin-5-yl)-3,8- diazabicyclo[3.2.1]octan-3-yl)methyl)piperidin- 1-yl)but-2-enamide |
| | 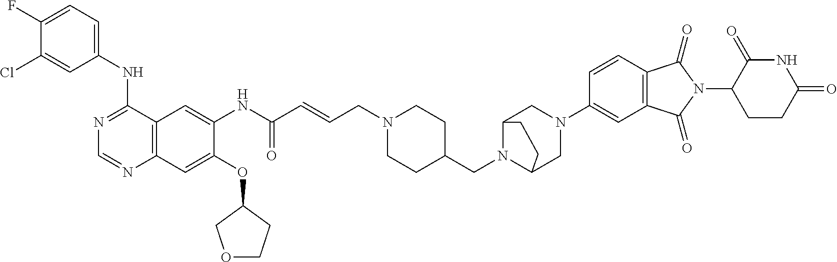 [SMILES: O=C(/C=C/CN1CCC(CN2C3CCC2CN(c2ccc4c(c2)C(=O)N(C2CCC(=O)NC2=O)C4=O)C3)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((3-(2-(2,6-dioxopiperidin-3-yl)-1,3- dioxoisoindolin-5-yl)-3,8- diazabicyclo[3.2.1]octan-8-yl)methyl)piperidin- 1-yl)but-2-enamide |
| | 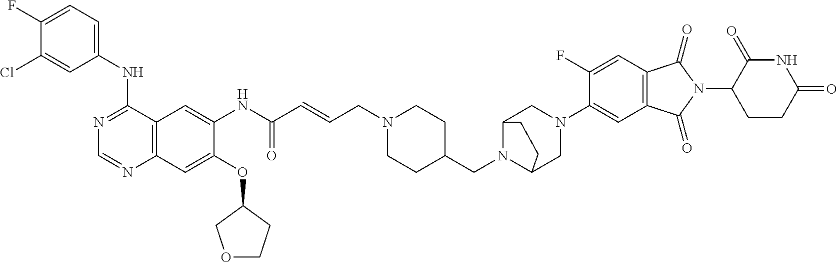 [SMILES: O=C(/C=C/CN1CCC(CN2C3CCC2CN(c2cc4c(cc2F)C(=O)N(C2CCC(=O)NC2=O)C4=O)C3)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((3-(2-(2,6-dioxopiperidin-3-yl)-6- fluoro-1,3-dioxoisoindolin-5-yl)-3,8- diazabicyclo[3.2.1]octan-8-yl)methyl)piperidin- 1-yl)but-2-enamide |
| | 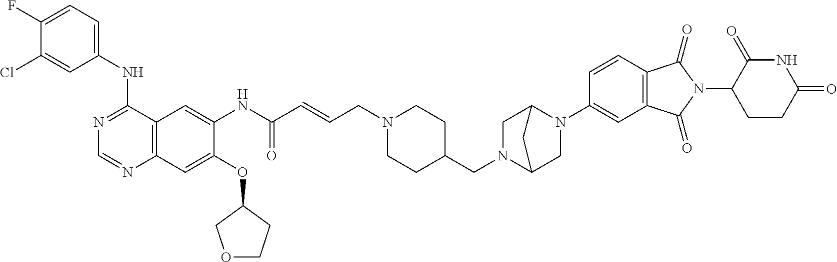 [SMILES: O=C(/C=C/CN1CCC(CN2CC3CC2CN3c2ccc3c(c2)C(=O)N(C2CCC(=O)NC2=O)C3=O)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((5-(2-(2,6-dioxopiperidin-3-yl)-1,3- dioxoisoindolin-5-yl)-2,5- diazabicyclo[2.2.1]heptan-2- yl)methyl)piperidin-1-yl)but-2-enamide |
| | 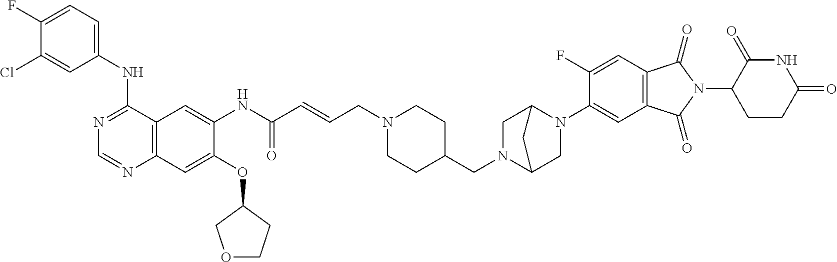 [SMILES: O=C(/C=C/CN1CCC(CN2CC3CC2CN3c2cc3c(cc2F)C(=O)N(C2CCC(=O)NC2=O)C3=O)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((5-(2-(2,6-dioxopiperidin-3-yl)-6- fluoro-1,3-dioxoisoindolin-5-yl)-2,5- diazabicyclo[2.2.1]heptan-2- yl)methyl)piperidin-1-yl)but-2-enamide |
| | 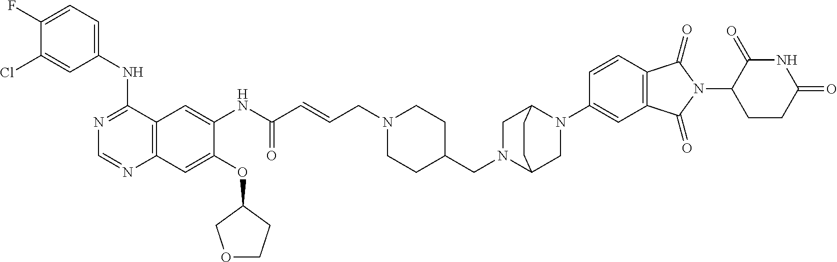 [SMILES: O=C(/C=C/CN1CCC(CN2CC3CCC2CN3c2ccc3c(c2)C(=O)N(C2CCC(=O)NC2=O)C3=O)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((5-(2-(2,6-dioxopiperidin-3-yl)-1,3- dioxoisoindolin-5-yl)-2,5- diazabicyclo[2.2.2]octan-2-yl)methyl)piperidin- 1-yl)but-2-enamide |
| | 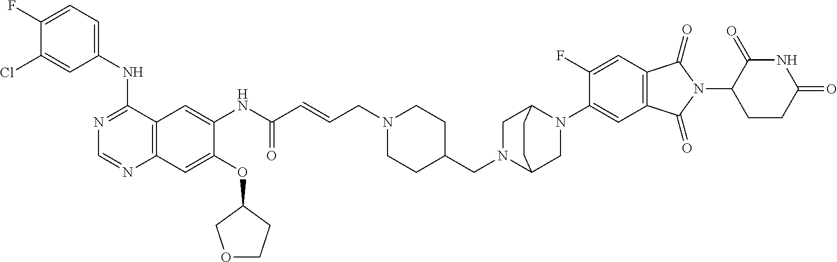 [SMILES: O=C(/C=C/CN1CCC(CN2CC3CCC2CN3c2cc3c(cc2F)C(=O)N(C2CCC(=O)NC2=O)C3=O)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((5-(2-(2,6-dioxopiperidin-3-yl)-6- fluoro-1,3-dioxoisoindolin-5-yl)-2,5- diazabicyclo[2.2.2]octan-2-yl)methyl)piperidin- 1-yl)but-2-enamide |
| | 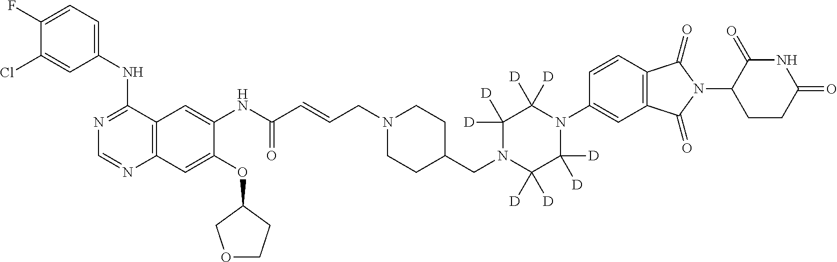 [SMILES: [2H]C1([2H])N(CC2CCN(C/C=C/C(=O)Nc3cc4c(Nc5ccc(F)c(Cl)c5)ncnc4cc3O[C@H]3CCOC3)CC2)C([2H])([2H])C([2H])([2H])N(c2ccc3c(c2)C(=O)N(C2CCC(=O)NC2=O)C3=O)C1([2H])[2H]] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-1,3- dioxoisoindolin-5-yl)piperazin-1-yl- 2,2,3,3,5,5,6,6-d₈)methyl)piperidin-1-yl)but-2- enamide |
| | 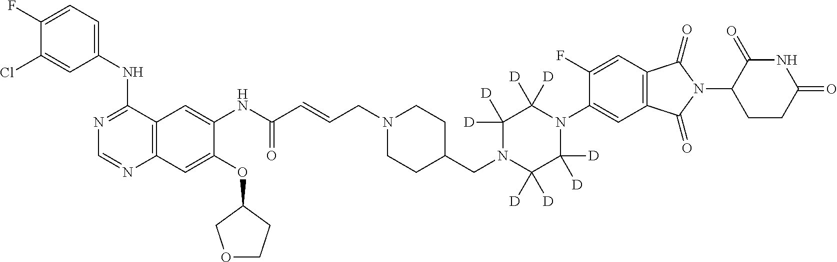 [SMILES: [2H]C1([2H])N(CC2CCN(C/C=C/C(=O)Nc3cc4c(Nc5ccc(F)c(Cl)c5)ncnc4cc3O[C@H]3CCOC3)CC2)C([2H])([2H])C([2H])([2H])N(c2cc3c(cc2F)C(=O)N(C2CCC(=O)NC2=O)C3=O)C1([2H])[2H]] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-6- fluoro-1,3-dioxoisoindolin-5-yl)piperazin-1-yl- 2,2,3,3,5,5,6,6-d₈)methyl)piperidin-1-yl)but-2- enamide |
| | 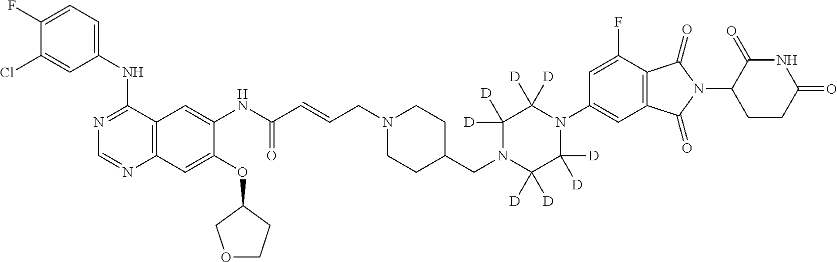 [SMILES: [2H]C1([2H])N(CC2CCN(C/C=C/C(=O)Nc3cc4c(Nc5ccc(F)c(Cl)c5)ncnc4cc3O[C@H]3CCOC3)CC2)C([2H])([2H])C([2H])([2H])N(c2cc(F)c3c(c2)C(=O)N(C2CCC(=O)NC2=O)C3=O)C1([2H])[2H]] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-7- fluoro-1,3-dioxoisoindolin-5-yl)piperazin-1-yl- 2,2,3,3,5,5,6,6-d₈)methyl)piperidin-1-yl)but-2- enamide |
| | 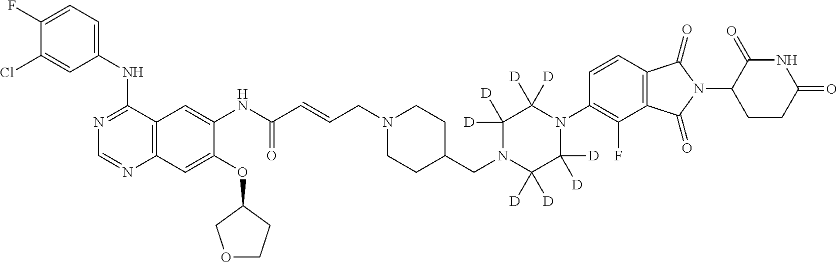 [SMILES: [2H]C1([2H])N(CC2CCN(C/C=C/C(=O)Nc3cc4c(Nc5ccc(F)c(Cl)c5)ncnc4cc3O[C@H]3CCOC3)CC2)C([2H])([2H])C([2H])([2H])N(c2ccc3c(c2F)C(=O)N(C2CCC(=O)NC2=O)C3=O)C1([2H])[2H]] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-4- fluoro-1,3-dioxoisoindolin-5-yl)piperazin-1-yl- 2,2,3,3,5,5,6,6-d₈)methyl)piperidin-1-yl)but-2- enamide |
| | 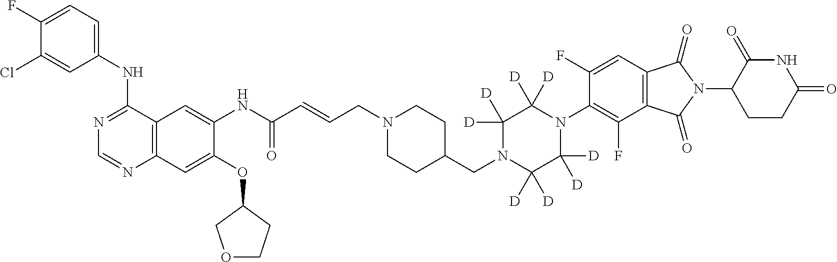 [SMILES: [2H]C1([2H])N(CC2CCN(C/C=C/C(=O)Nc3cc4c(Nc5ccc(F)c(Cl)c5)ncnc4cc3O[C@H]3CCOC3)CC2)C([2H])([2H])C([2H])([2H])N(c2c(F)cc3c(c2F)C(=O)N(C2CCC(=O)NC2=O)C3=O)C1([2H])[2H]] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-4,6- difluoro-1,3-dioxoisoindolin-5-yl)piperazin-1- yl-2,2,3,3,5,5,6,6-d₈)methyl)piperidin-1-yl)but- 2-enamide |
| | 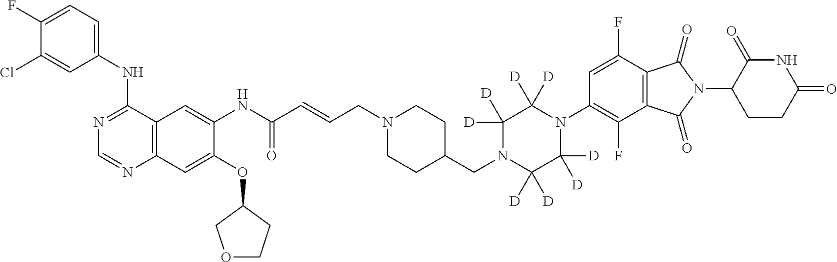 [SMILES: [2H]C1([2H])N(CC2CCN(C/C=C/C(=O)Nc3cc4c(Nc5ccc(F)c(Cl)c5)ncnc4cc3O[C@H]3CCOC3)CC2)C([2H])([2H])C([2H])([2H])N(c2cc(F)c3c(c2F)C(=O)N(C2CCC(=O)NC2=O)C3=O)C1([2H])[2H]] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-4,7- difluoro-1,3-dioxoisoindolin-5-yl)piperazin-1- yl-2,2,3,3,5,5,6,6-d₈)methyl)piperidin-1-yl)but- 2-enamide |
| | 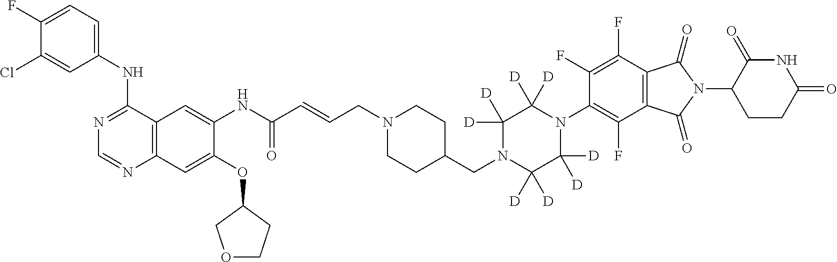 [SMILES: [2H]C1([2H])N(CC2CCN(C/C=C/C(=O)Nc3cc4c(Nc5ccc(F)c(Cl)c5)ncnc4cc3O[C@H]3CCOC3)CC2)C([2H])([2H])C([2H])([2H])N(c2c(F)c(F)c3c(c2F)C(=O)N(C2CCC(=O)NC2=O)C3=O)C1([2H])[2H]] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-4,6,7- trifluoro-1,3-dioxoisoindolin-5-yl)piperazin-1 - yl-2,2,3,3,5,5,6,6-ds)methyl)piperidin-1-yl)but- 2-enamide |
| | 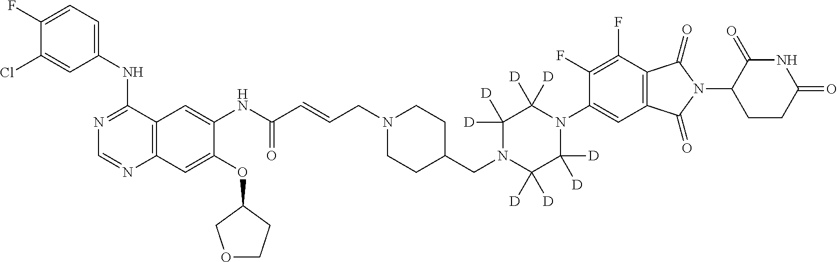 [SMILES: [2H]C1([2H])N(CC2CCN(C/C=C/C(=O)Nc3cc4c(Nc5ccc(F)c(Cl)c5)ncnc4cc3O[C@H]3CCOC3)CC2)C([2H])([2H])C([2H])([2H])N(c2cc3c(c(F)c2F)C(=O)N(C2CCC(=O)NC2=O)C3=O)C1([2H])[2H]] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-6,7- difluoro-1,3-dioxoisoindolin-5-yl)piperazin-1- yl-2,2,3,3,5,5,6,6-d₈)methyl)piperidin-1-yl)but- 2-enamide |
| | 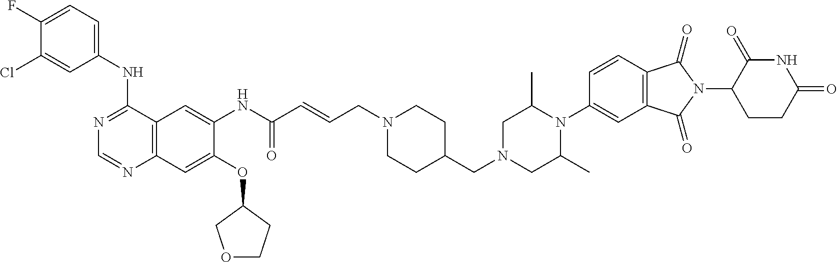 [SMILES: CC1CN(CC2CCN(C/C=C/C(=O)Nc3cc4c(Nc5ccc(F)c(Cl)c5)ncnc4cc3O[C@H]3CCOC3)CC2)CC(C)N1c1ccc2c(c1)C(=O)N(C1CCC(=O)NC1=O)C2=O] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-1,3- dioxoisoindolin-5-yl)-3,5-dimethylpiperazin-1- yl)methyl)piperidin-1-yl)but-2-enamide |
| | 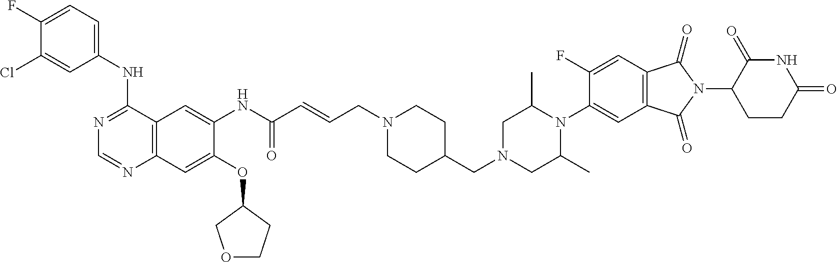 [SMILES: CC1CN(CC2CCN(C/C=C/C(=O)Nc3cc4c(Nc5ccc(F)c(Cl)c5)ncnc4cc3O[C@H]3CCOC3)CC2)CC(C)N1c1cc2c(cc1F)C(=O)N(C1CCC(=O)NC1=O)C2=O] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-6- fluoro-1,3-dioxoisoindolin-5-yl)-3,5- dimethylpiperazin-1-yl)methyl)piperidin-1- yl)but-2-enamide |
| | 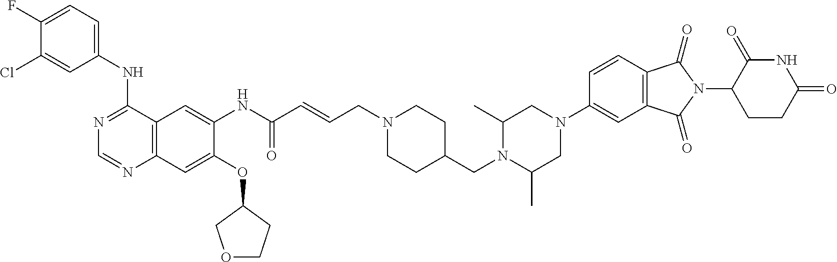 [SMILES: CC1CN(c2ccc3c(c2)C(=O)N(C2CCC(=O)NC2=O)C3=O)CC(C)N1CC1CCN(C/C=C/C(=O)Nc2cc3c(Nc4ccc(F)c(Cl)c4)ncnc3cc2O[C@H]2CCOC2)CC1] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-1,3- dioxoisoindolin-5-yl)-2,6-dimethylpiperazin-1- yl)methyl)piperidin-1-yl)but-2-enamide |
| | 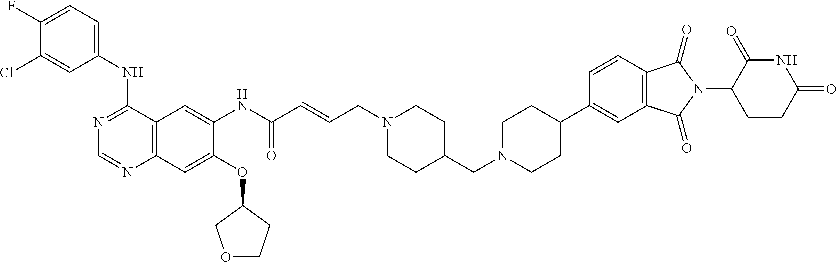 [SMILES: O=C(/C=C/CN1CCC(CN2CCC(c3ccc4c(c3)C(=O)N(C3CCC(=O)NC3=O)C4=O)CC2)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-1,3- dioxoisoindolin-5-yl)piperidin-1- yl)methyl)piperidin-1-yl)but-2-enamide |
| | 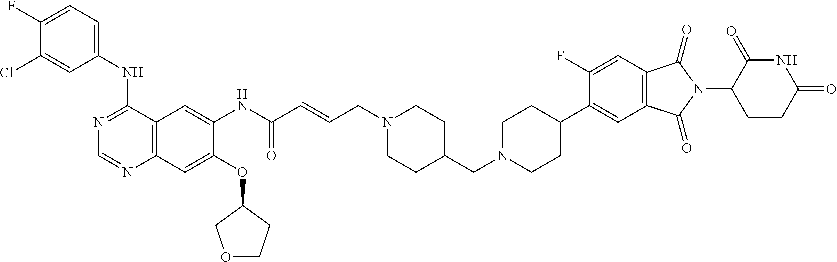 [SMILES: O=C(/C=C/CN1CCC(CN2CCC(c3cc4c(cc3F)C(=O)N(C3CCC(=O)NC3=O)C4=O)CC2)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-6- fluoro-1,3-dioxoisoindolin-5-yl)piperidin-1- yl)methyl)piperidin-1-yl)but-2-enamide |
| | 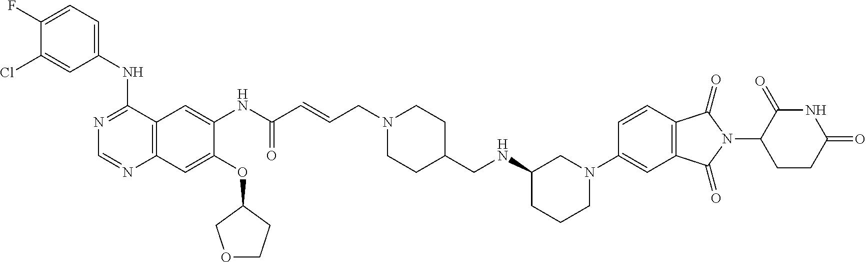 [SMILES: O=C(/C=C/CN1CCC(CN[C@@H]2CCCN(c3ccc4c(c3)C(=O)N(C3CCC(=O)NC3=O)C4=O)C2)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((((3R)-1-(2-(2,6-dioxopiperidin-3-yl)- 1,3-dioxoisoindolin-5-yl)piperidin-3- yl)amino)methyl)piperidin-1-yl)but-2-enamide |
| | 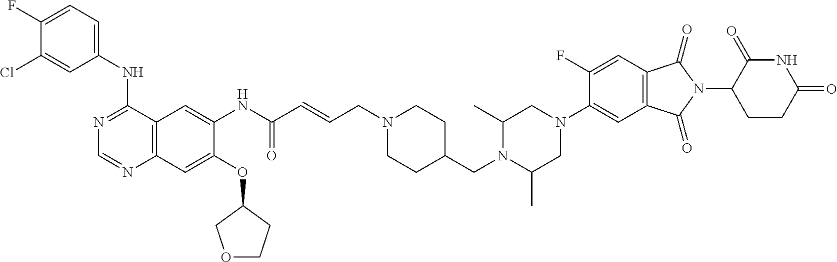 [SMILES: CC1CN(c2cc3c(cc2F)C(=O)N(C2CCC(=O)NC2=O)C3=O)CC(C)N1CC1CCN(C/C=C/C(=O)Nc2cc3c(Nc4ccc(F)c(Cl)c4)ncnc3cc2O[C@H]2CCOC2)CC1] | (E)-N-(4-((3-chloro-4-fluorophenyl)amino)-7- (((S)-tetrahydrofuran-3-yl)oxy)quinazolin-6- yl)-4-(4-((4-(2-(2,6-dioxopiperidin-3-yl)-6- fluoro-1,3-dioxoisoindolin-5-yl)-2,6- dimethylpiperazin-1-yl)methyl)piperidin-1- yl)but-2-enamide |
| | 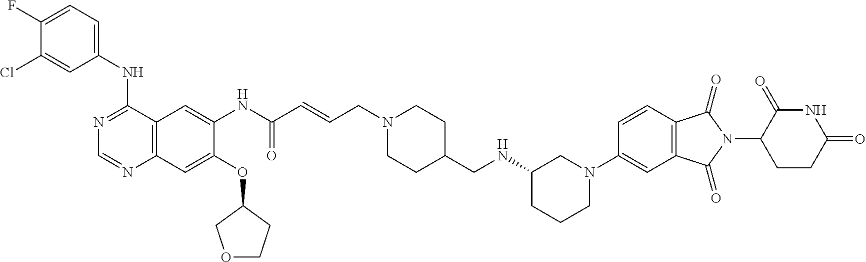 [SMILES: O=C(/C=C/CN1CCC(CN[C@H]2CCCN(c3ccc4c(c3)C(=O)N(C3CCC(=O)NC3=O)C4=O)C2)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-((((3S)-1-(2-(2,6-dioxopiperidin-3-yl)- 1,3-dioxoisoindolin-5-yl)piperidin-3- yl)amino)methyl)piperidin-1-yl)but-2-enamide |
| | 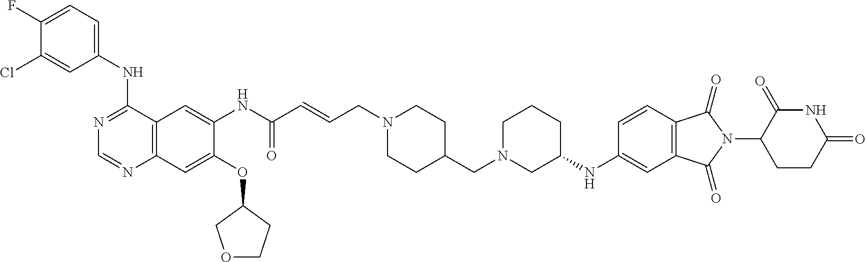 [SMILES: O=C(/C=C/CN1CCC(CN2CCC[C@H](Nc3ccc4c(c3)C(=O)N(C3CCC(=O)NC3=O)C4=O)C2)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-(((3S)-3-((2-(2,6-dioxopiperidin-3-yl)- 1,3-dioxoisoindolin-5-yl)amino)piperidin-1- yl)methyl)piperidin-1-yl)but-2-enamide |
| | 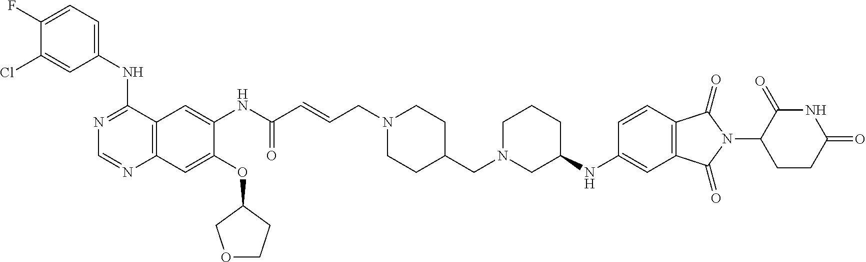 [SMILES: O=C(/C=C/CN1CCC(CN2CCC[C@@H](Nc3ccc4c(c3)C(=O)N(C3CCC(=O)NC3=O)C4=O)C2)CC1)Nc1cc2c(Nc3ccc(F)c(Cl)c3)ncnc2cc1O[C@H]1CCOC1] | (E)-N-(4-((3-chloro-4-fluorophenyl)amino)-7- (((S)-tetrahydrofuran-3-yl)oxy)quinazolin-6- yl)-4-(4-(((3R)-3-((2-(2,6-dioxopiperidin-3-yl)- 1,3-dioxoisoindolin-5-yl)amino)piperidin-1- yl)methyl)piperidin-1-yl)but-2-enamide |
| | 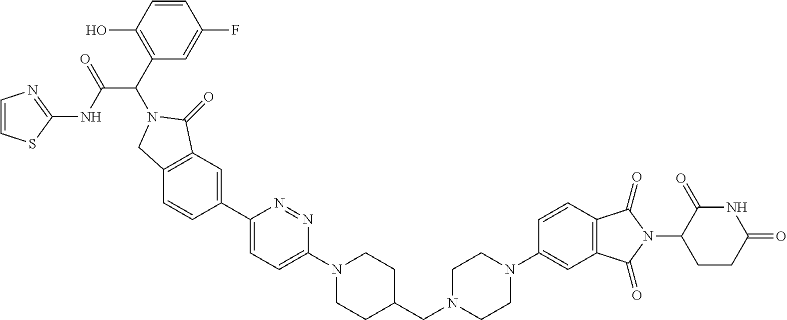 [SMILES: O=C1CCC(N2C(=O)c3ccc(N4CCN(CC5CCN(c6ccc(-c7ccc8c(c7)C(=O)N(C(C(=O)Nc7nccs7)c7cc(F)ccc7O)C8)nn6)CC5)CC4)cc3C2=O)C(=O)N1] | 2-(6-(6-(4-((4-(2-(2,6-dioxopiperidin-3-yl)-1,3- dioxoisoindolin-5-yl)piperazin-1- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 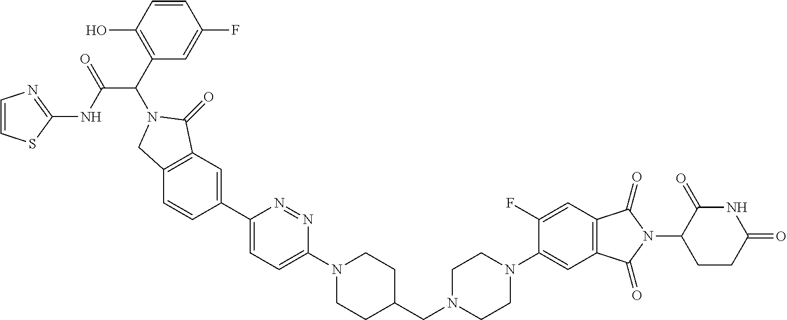 [SMILES: O=C1CCC(N2C(=O)c3cc(F)c(N4CCN(CC5CCN(c6ccc(-c7ccc8c(c7)C(=O)N(C(C(=O)Nc7nccs7)c7cc(F)ccc7O)C8)nn6)CC5)CC4)cc3C2=O)C(=O)N1] | 2-(6-(6-(4-((4-(2-(2,6-dioxopiperidin-3-yl)-6- fluoro-1,3-dioxoisoindolin-5-yl)piperazin-1- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 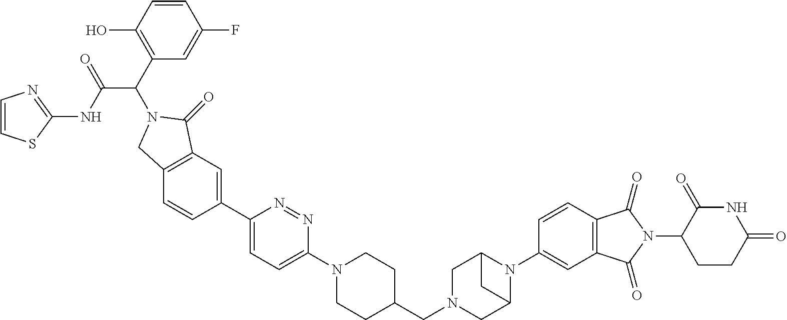 [SMILES: O=C1CCC(N2C(=O)c3ccc(N4C5CC4CN(CC4CCN(c6ccc(-c7ccc8c(c7)C(=O)N(C(C(=O)Nc7nccs7)c7cc(F)ccc7O)C8)nn6)CC4)C5)cc3C2=O)C(=O)N1] | 2-(6-(6-(4-((6-(2-(2,6-dioxopiperidin-3-yl)-1,3- dioxoisoindolin-5-yl)-3,6- diazabicyclo[3.1.1]heptan-3- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 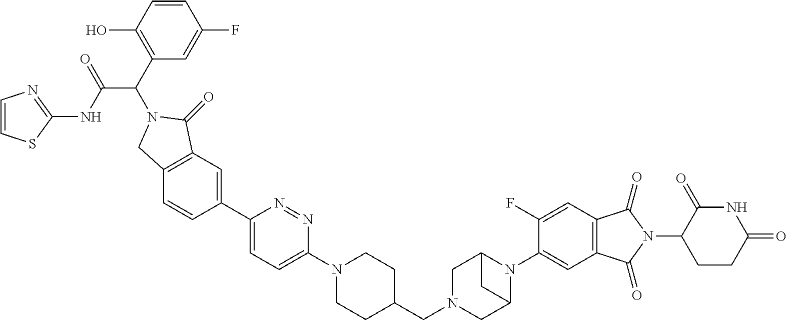 [SMILES: O=C1CCC(N2C(=O)c3cc(F)c(N4C5CC4CN(CC4CCN(c6ccc(-c7ccc8c(c7)C(=O)N(C(C(=O)Nc7nccs7)c7cc(F)ccc7O)C8)nn6)CC4)C5)cc3C2=O)C(=O)N1] | 2-(6-(6-(4-((6-(2-(2,6-dioxopiperidin-3-yl)-6- fluoro-1,3-dioxoisoindolin-5-yl)-3,6- diazabicyclo[3.1.1]heptan-3- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 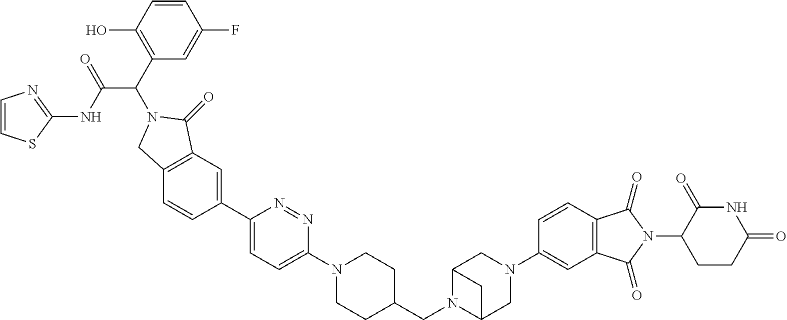 [SMILES: O=C1CCC(N2C(=O)c3ccc(N4CC5CC(C4)N5CC4CCN(c5ccc(-c6ccc7c(c6)C(=O)N(C(C(=O)Nc6nccs6)c6cc(F)ccc6O)C7)nn5)CC4)cc3C2=O)C(=O)N1] | 2-(6-(6-(4-((3-(2-(2,6-dioxopiperidin-3-yl)-1,3- dioxoisoindolin-5-yl)-3,6- diazabicyclo[3.1.1]heptan-6- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 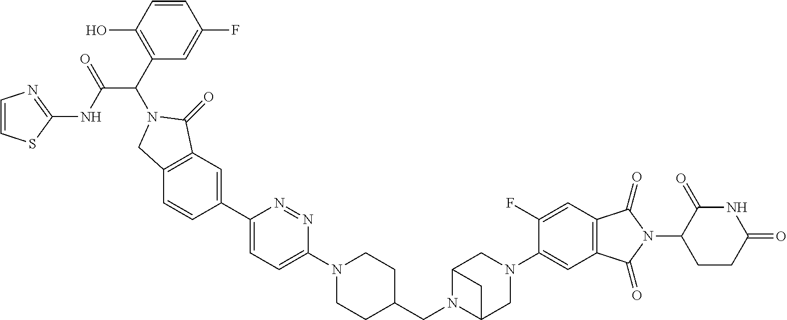 [SMILES: O=C1CCC(N2C(=O)c3cc(F)c(N4CC5CC(C4)N5CC4CCN(c5ccc(-c6ccc7c(c6)C(=O)N(C(C(=O)Nc6nccs6)c6cc(F)ccc6O)C7)nn5)CC4)cc3C2=O)C(=O)N1] | 2-(6-(6-(4-((3-(2-(2,6-dioxopiperidin-3-yl)-6- fluoro-1,3-dioxoisoindolin-5-yl)-3,6- diazabicyclo[3.1.1]heptan-6- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 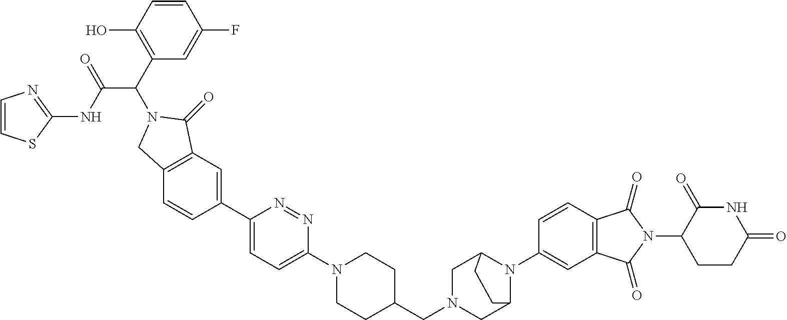 [SMILES: O=C1CCC(N2C(=O)c3ccc(N4C5CCC4CN(CC4CCN(c6ccc(-c7ccc8c(c7)C(=O)N(C(C(=O)Nc7nccs7)c7cc(F)ccc7O)C8)nn6)CC4)C5)cc3C2=O)C(=O)N1] | 2-(6-(6-(4-((8-(2-(2,6-dioxopiperidin-3-yl)-1,3- dioxoisoindolin-5-yl)-3,8- diazabicyclo[3.2.1]octan-3-yl)methyl)piperidin- 1-yl)pyridazin-3-yl)-1-oxoisoindolin-2-yl)-2- (5-fluoro-2-hydroxyphenyl)-N-(thiazol-2- yl)acetamide |
| | 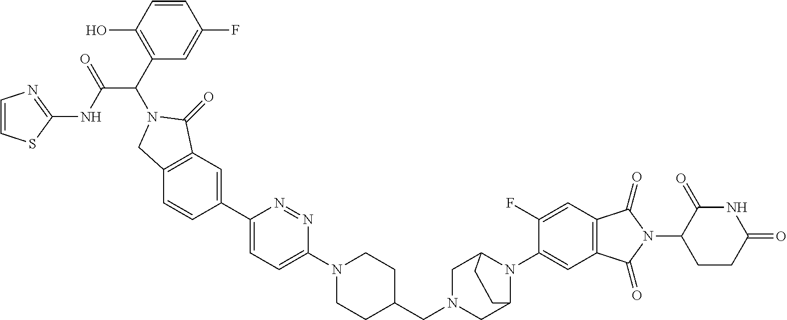 [SMILES: O=C1CCC(N2C(=O)c3cc(F)c(N4C5CCC4CN(CC4CCN(c6ccc(-c7ccc8c(c7)C(=O)N(C(C(=O)Nc7nccs7)c7cc(F)ccc7O)C8)nn6)CC4)C5)cc3C2=O)C(=O)N1] | 2-(6-(6-(4-((8-(2-(2,6-dioxopiperidin-3-yl)-6- fluoro-1,3-dioxoisoindolin-5-yl)-3,8- diazabicyclo[3.2.1]octan-3-yl)methyl)piperidin- 1-yl)pyridazin-3-yl)-1-oxoisoindolin-2-yl)-2- (5-fluoro-2-hydroxyphenyl)-N-(thiazol-2- yl)acetamide |
| | 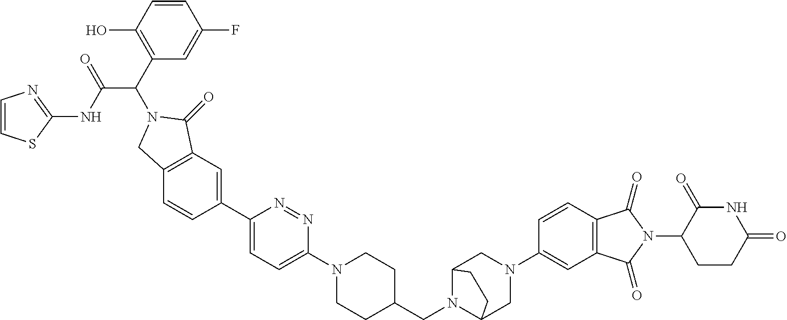 [SMILES: O=C1CCC(N2C(=O)c3ccc(N4CC5CCC(C4)N5CC4CCN(c5ccc(-c6ccc7c(c6)C(=O)N(C(C(=O)Nc6nccs6)c6cc(F)ccc6O)C7)nn5)CC4)cc3C2=O)C(=O)N1] | 2-(6-(6-(4-((3-(2-(2,6-dioxopiperidin-3-yl)-1,3- dioxoisoindolin-5-yl)-3,8- diazabicyclo[3.2.1]octan-8-yl)methyl)piperidin- 1-yl)pyridazin-3-yl)-1-oxoisoindolin-2-yl)-2- (5-fluoro-2-hydroxyphenyl)-N-(thiazol-2- yl)acetamide |
| | 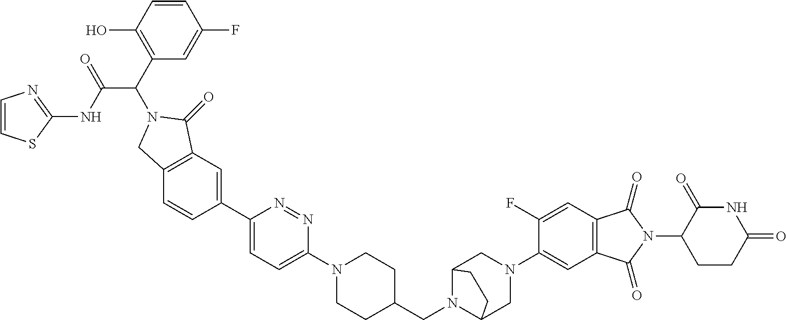 [SMILES: O=C1CCC(N2C(=O)c3cc(F)c(N4CC5CCC(C4)N5CC4CCN(c5ccc(-c6ccc7c(c6)C(=O)N(C(C(=O)Nc6nccs6)c6cc(F)ccc6O)C7)nn5)CC4)cc3C2=O)C(=O)N1] | 2-(6-(6-(4-((3-(2-(2,6-dioxopiperidin-3-yl)-6- fluoro-1,3-dioxoisoindolin-5-yl)-3,8- diazabicyclo[3.2.1]octan-8-yl)methyl)piperidin- 1-yl)pyridazin-3-yl)-1-oxoisoindolin-2-yl)-2- (5-fluoro-2-hydroxyphenyl)-N-(thiazol-2- yl)acetamide |
| | 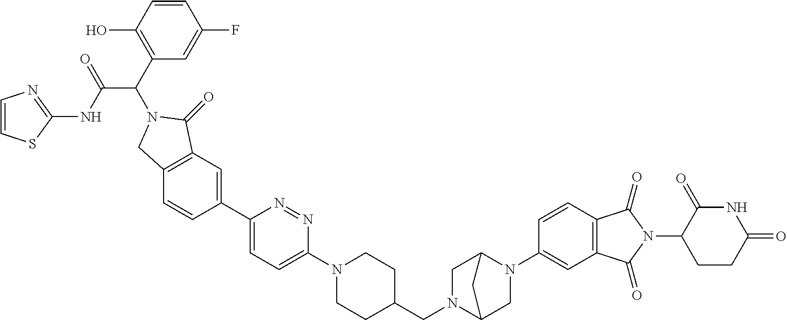 [SMILES: O=C1CCC(N2C(=O)c3ccc(N4CC5CC4CN5CC4CCN(c5ccc(-c6ccc7c(c6)C(=O)N(C(C(=O)Nc6nccs6)c6cc(F)ccc6O)C7)nn5)CC4)cc3C2=O)C(=O)N1] | 2-(6-(6-(4-((5-(2-(2,6-dioxopiperidin-3-yl)-1,3- dioxoisoindolin-5-yl)-2,5- diazabicyclo[2.2.1]heptan-2- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 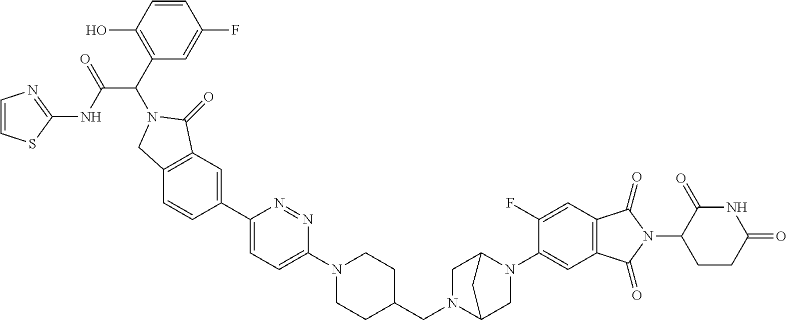 [SMILES: O=C1CCC(N2C(=O)c3cc(F)c(N4CC5CC4CN5CC4CCN(c5ccc(-c6ccc7c(c6)C(=O)N(C(C(=O)Nc6nccs6)c6cc(F)ccc6O)C7)nn5)CC4)cc3C2=O)C(=O)N1] | 2-(6-(6-(4-((5-(2-(2,6-dioxopiperidin-3-yl)-6- fluoro-1,3-dioxoisoindolin-5-yl)-2,5- diazabicyclo[2.2.1]heptan-2- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 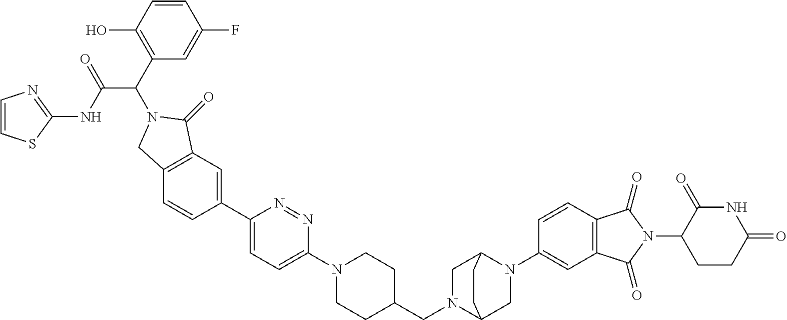 [SMILES: O=C1CCC(N2C(=O)c3ccc(N4CC5CCC4CN5CC4CCN(c5ccc(-c6ccc7c(c6)C(=O)N(C(C(=O)Nc6nccs6)c6cc(F)ccc6O)C7)nn5)CC4)cc3C2=O)C(=O)N1] | 2-(6-(6-(4-((5-(2-(2,6-dioxopiperidin-3-yl)-1,3- dioxoisoindolin-5-yl)-2,5- diazabicyclo[2.2.2]octan-2-yl)methyl)piperidin- 1-yl)pyridazin-3-yl)-1-oxoisoindolin-2-yl)-2- (5-fluoro-2-hydroxyphenyl)-N-(thiazol-2- yl)acetamide |
| | 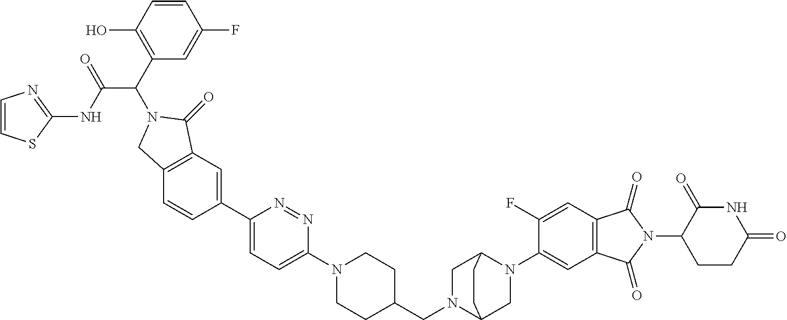 [SMILES: O=C1CCC(N2C(=O)c3cc(F)c(N4CC5CCC4CN5CC4CCN(c5ccc(-c6ccc7c(c6)C(=O)N(C(C(=O)Nc6nccs6)c6cc(F)ccc6O)C7)nn5)CC4)cc3C2=O)C(=O)N1] | 2-(6-(6-(4-((5-(2-(2,6-dioxopiperidin-3-yl)-6- fluoro-1,3-dioxoisoindolin-5-yl)-2,5- diazabicyclo[2.2.2]octan-2-yl)methyl)piperidin- 1-yl)pyridazin-3-yl)-1-oxoisoindolin-2-yl)-2- (5-fluoro-2-hydroxyphenyl)-N-(thiazol-2- yl)acetamide |
| | 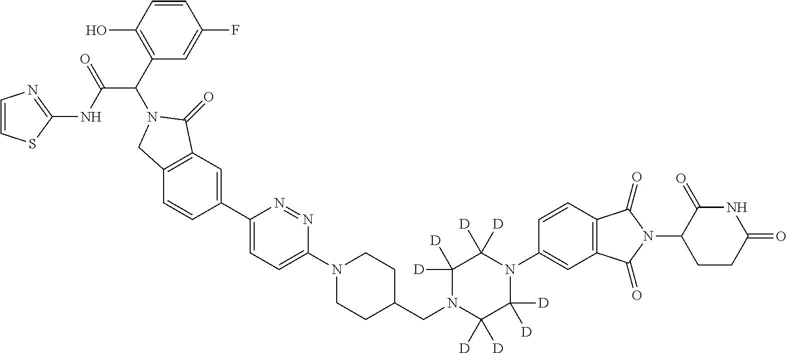 [SMILES: [2H]C1([2H])N(CC2CCN(c3ccc(-c4ccc5c(c4)C(=O)N(C(C(=O)Nc4nccs4)c4cc(F)ccc4O)C5)nn3)CC2)C([2H])([2H])C([2H])([2H])N(c2ccc3c(c2)C(=O)N(C2CCC(=O)NC2=O)C3=O)C1([2H])[2H]] | 2-(6-(6-(4-((4-(2-(2,6-dioxopiperidin-3-yl)-1,3- dioxoisoindolin-5-yl)piperazin-1-yl- 2,2,3,3,5,5,6,6-d₈)methyl)piperidin-1- yl)pyridazin-3-yl)-1-oxoisoindolin-2-yl)-2-(5- fluoro-2-hydroxyphenyl)-N-(thiazol-2- yl)acetamide |
| | 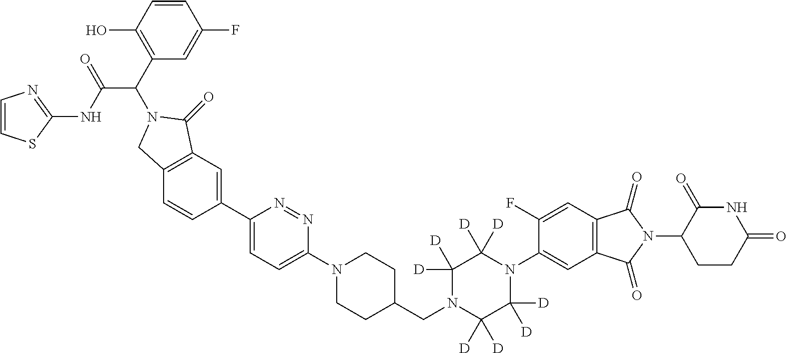 [SMILES: [2H]C1([2H])N(CC2CCN(c3ccc(-c4ccc5c(c4)C(=O)N(C(C(=O)Nc4nccs4)c4cc(F)ccc4O)C5)nn3)CC2)C([2H])([2H])C([2H])([2H])N(c2cc3c(cc2F)C(=O)N(C2CCC(=O)NC2=O)C3=O)C1([2H])[2H]] | 2-(6-(6-(4-((4-(2-(2,6-dioxopiperidin-3-yl)-6- fluoro-1,3-dioxoisoindolin-5-yl)piperazin-1-yl- 2,2,3,3,5,5,6,6-d₈)methyl)piperidin-1- yl)pyridazin-3-yl)-1-oxoisoindolin-2-yl)-2-(5- fluoro-2-hydroxyphenyl)-N-(thiazol-2- yl)acetamide |
| | 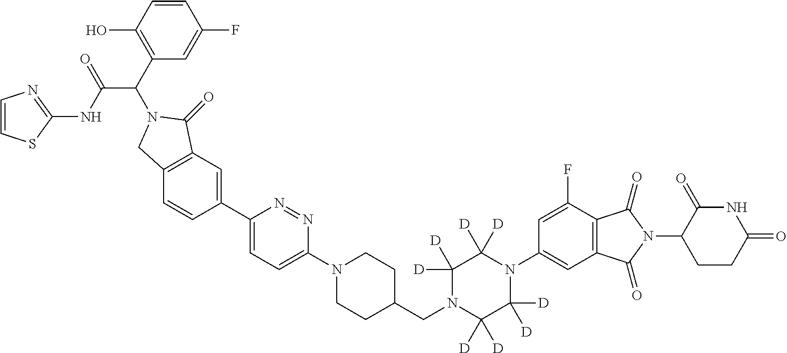 [SMILES: [2H]C1([2H])N(CC2CCN(c3ccc(-c4ccc5c(c4)C(=O)N(C(C(=O)Nc4nccs4)c4cc(F)ccc4O)C5)nn3)CC2)C([2H])([2H])C([2H])([2H])N(c2cc(F)c3c(c2)C(=O)N(C2CCC(=O)NC2=O)C3=O)C1([2H])[2H]] | 2-(6-(6-(4-((4-(2-(2,6-dioxopiperidin-3-yl)-7- fluoro-1,3-dioxoisoindolin-5-yl)piperazin-1-yl- 2,2,3,3,5,5,6,6-d₈)methyl)piperidin-1- yl)pyridazin-3-yl)-1-oxoisoindolin-2-yl)-2-(5- fluoro-2-hydroxyphenyl)-N-(thiazol-2- yl)acetamide |
| | 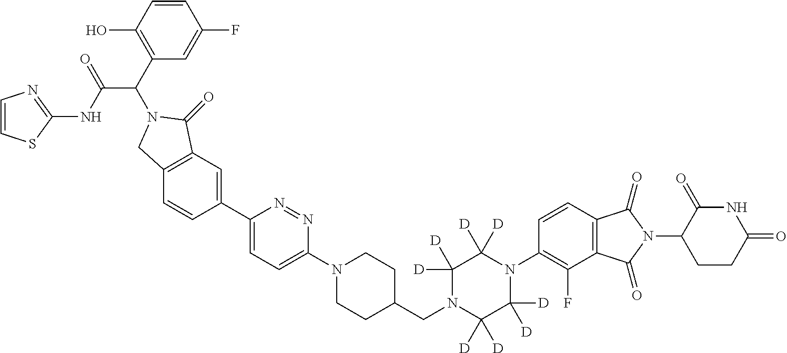 [SMILES: [2H]C1([2H])N(CC2CCN(c3ccc(-c4ccc5c(c4)C(=O)N(C(C(=O)Nc4nccs4)c4cc(F)ccc4O)C5)nn3)CC2)C([2H])([2H])C([2H])([2H])N(c2ccc3c(c2F)C(=O)N(C2CCC(=O)NC2=O)C3=O)C1([2H])[2H]] | 2-(6-(6-(4-((4-(2-(2,6-dioxopiperidin-3-yl)-4- fluoro-1,3-dioxoisoindolin-5-yl)piperazin-1-yl- 2,2,3,3,5,5,6,6-d₈)methyl)piperidin-1- yl)pyridazin-3-yl)-1-oxoisoindolin-2-yl)-2-(5- fluoro-2-hydroxyphenyl)-N-(thiazol-2- yl)acetamide |
| | 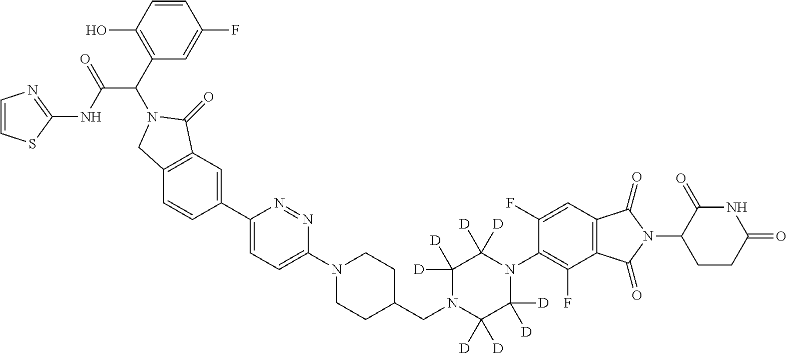 [SMILES: [2H]C1([2H])N(CC2CCN(c3ccc(-c4ccc5c(c4)C(=O)N(C(C(=O)Nc4nccs4)c4cc(F)ccc4O)C5)nn3)CC2)C([2H])([2H])C([2H])([2H])N(c2c(F)cc3c(c2F)C(=O)N(C2CCC(=O)NC2=O)C3=O)C1([2H])[2H]] | 2-(6-(6-(4-((4-(2-(2,6-dioxopiperidin-3-yl)-4,6- difluoro-1,3-dioxoisoindolin-5-yl)piperazin-1- yl-2,2,3,3,5,5,6,6-d₈)methyl)piperidin-1- yl)pyridazin-3-yl)-1-oxoisoindolin-2-yl)-2-(5- fluoro-2-hydroxyphenyl)-N-(thiazol-2- yl)acetamide |
| | 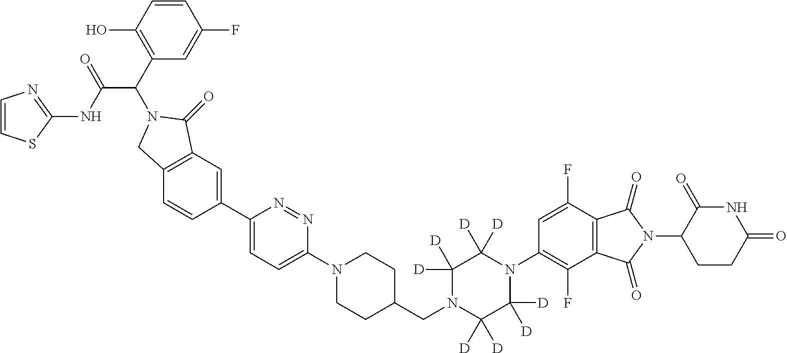 [SMILES: [2H]C1([2H])N(CC2CCN(c3ccc(-c4ccc5c(c4)C(=O)N(C(C(=O)Nc4nccs4)c4cc(F)ccc4O)C5)nn3)CC2)C([2H])([2H])C([2H])([2H])N(c2cc(F)c3c(c2F)C(=O)N(C2CCC(=O)NC2=O)C3=O)C1([2H])[2H]] | 2-(6-(6-(4-((4-(2-(2,6-dioxopiperidin-3-yl)-4,7- difluoro-1,3-dioxoisoindolin-5-yl)piperazin-1- yl-2,2,3,3,5,5,6,6-d₈)methyl)piperidin-1- yl)pyridazin-3-yl)-1-oxoisoindolin-2-yl)-2-(5- fluoro-2-hydroxyphenyl)-N-(thiazol-2- yl)acetamide |
| | 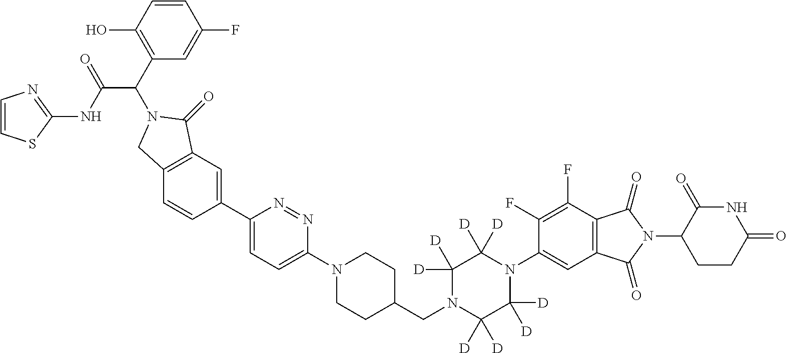 [SMILES: [2H]C1([2H])N(CC2CCN(c3ccc(-c4ccc5c(c4)C(=O)N(C(C(=O)Nc4nccs4)c4cc(F)ccc4O)C5)nn3)CC2)C([2H])([2H])C([2H])([2H])N(c2cc3c(c(F)c2F)C(=O)N(C2CCC(=O)NC2=O)C3=O)C1([2H])[2H]] | 2-(6-(6-(4-((4-(2-(2,6-dioxopiperidin-3-yl)-6,7- difluoro-1,3-dioxoisoindolin-5-yl)piperazin-1- yl-2,2,3,3,5,5,6,6-d₈)methyl)piperidin-1- yl)pyridazin-3-yl)-1-oxoisoindolin-2-yl)-2-(5- fluoro-2-hydroxyphenyl)-N-(thiazol-2- yl)acetamide |
| | 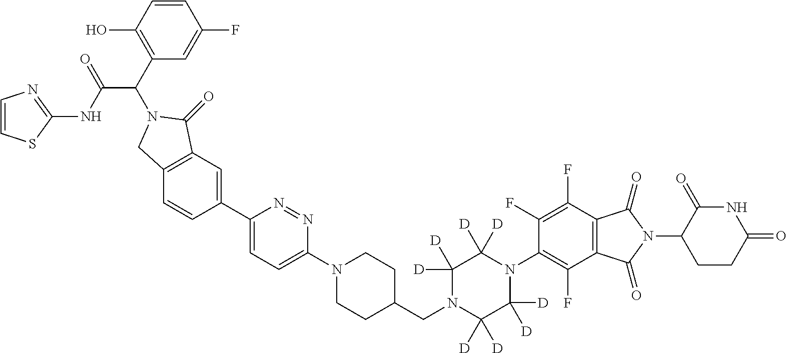 [SMILES: [2H]C1([2H])N(CC2CCN(c3ccc(-c4ccc5c(c4)C(=O)N(C(C(=O)Nc4nccs4)c4cc(F)ccc4O)C5)nn3)CC2)C([2H])([2H])C([2H])([2H])N(c2c(F)c(F)c3c(c2F)C(=O)N(C2CCC(=O)NC2=O)C3=O)C1([2H])[2H]] | 2-(6-(6-(4-((4-(2-(2,6-dioxopiperidin-3-yl)- 4,6,7-trifluoro-1,3-dioxoisoindolin-5- yl)piperazin-1-yl-2,2,3,3,5,5,6,6- d₈)methyl)piperidin-1-yl)pyridazin-3-yl)-1- oxoisoindolin-2-yl)-2-(5-fluoro-2- hydroxyphenyl)-N-(thiazol-2-yl)acetamide |
| | 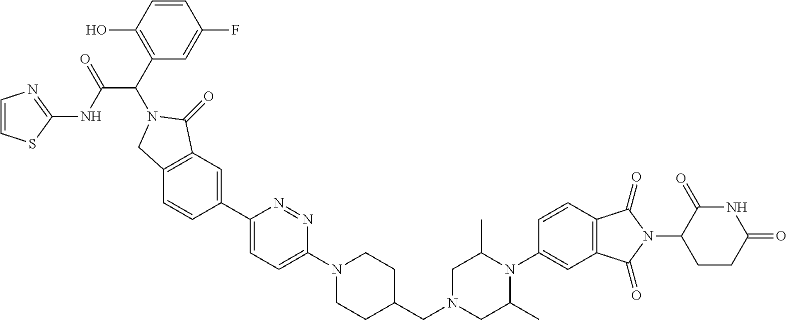 [SMILES: CC1CN(CC2CCN(c3ccc(-c4ccc5c(c4)C(=O)N(C(C(=O)Nc4nccs4)c4cc(F)ccc4O)C5)nn3)CC2)CC(C)N1c1ccc2c(c1)C(=O)N(C1CCC(=O)NC1=O)C2=O] | 2-(6-(6-(4-((4-(2-(2,6-dioxopiperidin-3-yl)-1,3- dioxoisoindolin-5-yl)-3,5-dimethylpiperazin-1- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 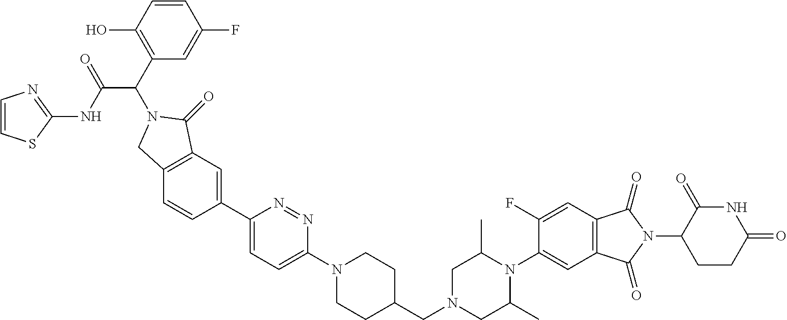 [SMILES: CC1CN(CC2CCN(c3ccc(-c4ccc5c(c4)C(=O)N(C(C(=O)Nc4nccs4)c4cc(F)ccc4O)C5)nn3)CC2)CC(C)N1c1cc2c(cc1F)C(=O)N(C1CCC(=O)NC1=O)C2=O] | 2-(6-(6-(4-((4-(2-(2,6-dioxopiperidin-3-yl)-6- fluoro-1,3-dioxoisoindolin-5-yl)-3,5- dimethylpiperazin-1-yl)methyl)piperidin-1- yl)pyridazin-3-yl)-1-oxoisoindolin-2-yl)-2-(5- fluoro-2-hydroxyphenyl)-N-(thiazol-2- yl)acetamide |
| | 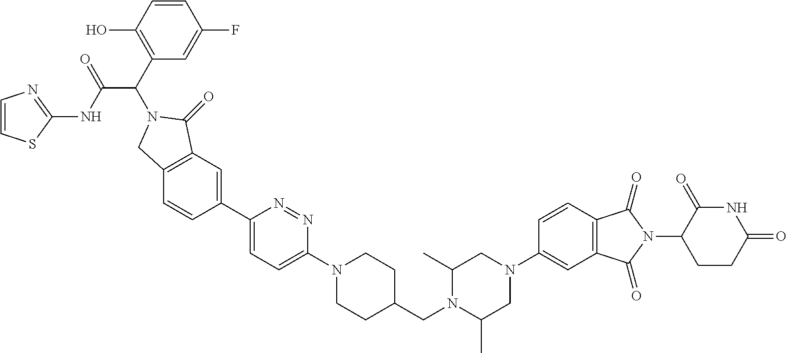 [SMILES: CC1CN(c2ccc3c(c2)C(=O)N(C2CCC(=O)NC2=O)C3=O)CC(C)N1CC1CCN(c2ccc(-c3ccc4c(c3)C(=O)N(C(C(=O)Nc3nccs3)c3cc(F)ccc3O)C4)nn2)CC1] | 2-(6-(6-(4-((4-(2-(2,6-dioxopiperidin-3-yl)-1,3- dioxoisoindolin-5-yl)-2,6-dimethylpiperazin-1- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 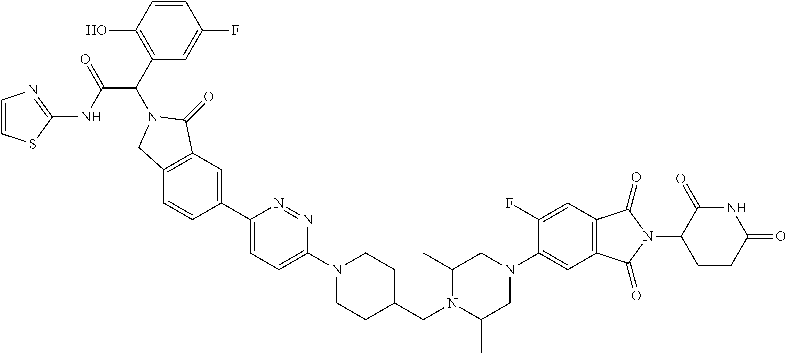 [SMILES: CC1CN(c2cc3c(cc2F)C(=O)N(C2CCC(=O)NC2=O)C3=O)CC(C)N1CC1CCN(c2ccc(-c3ccc4c(c3)C(=O)N(C(C(=O)Nc3nccs3)c3cc(F)ccc3O)C4)nn2)CC1] | 2-(6-(6-(4-((4-(2-(2,6-dioxopiperidin-3-yl)-6- fluoro-1,3-dioxoisoindolin-5-yl)-2,6- dimethylpiperazin-1-yl)methyl)piperidin-1- yl)pyridazin-3-yl)-1-oxoisoindolin-2-yl)-2-(5- fluoro-2-hydroxyphenyl)-N-(thiazol-2- yl)acetamide |
| | 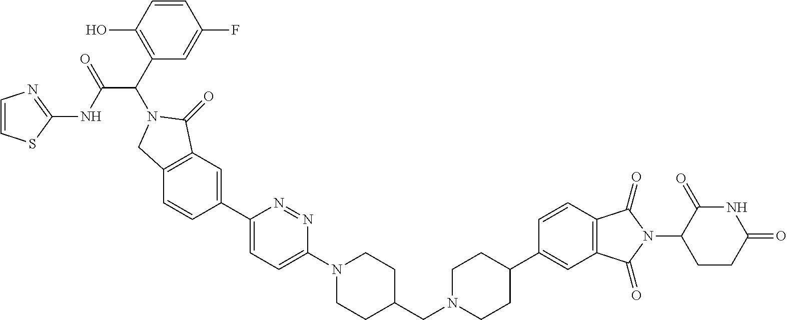 [SMILES: O=C1CCC(N2C(=O)c3ccc(C4CCN(CC5CCN(c6ccc(-c7ccc8c(c7)C(=O)N(C(C(=O)Nc7nccs7)c7cc(F)ccc7O)C8)nn6)CC5)CC4)cc3C2=O)C(=O)N1] | 2-(6-(6-(4-((4-(2-(2,6-dioxopiperidin-3-yl)-1,3- dioxoisoindolin-5-yl)piperidin-1- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 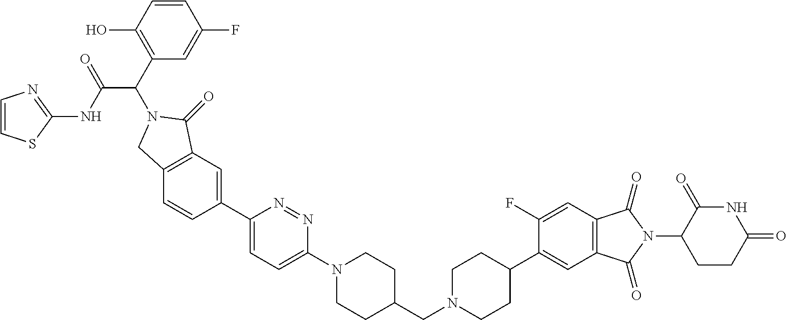 [SMILES: O=C1CCC(N2C(=O)c3cc(F)c(C4CCN(CC5CCN(c6ccc(-c7ccc8c(c7)C(=O)N(C(C(=O)Nc7nccs7)c7cc(F)ccc7O)C8)nn6)CC5)CC4)cc3C2=O)C(=O)N1] | 2-(6-(6-(4-((4-(2-(2,6-dioxopiperidin-3-yl)-6- fluoro-1,3-dioxoisoindolin-5-yl)piperidin-1- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 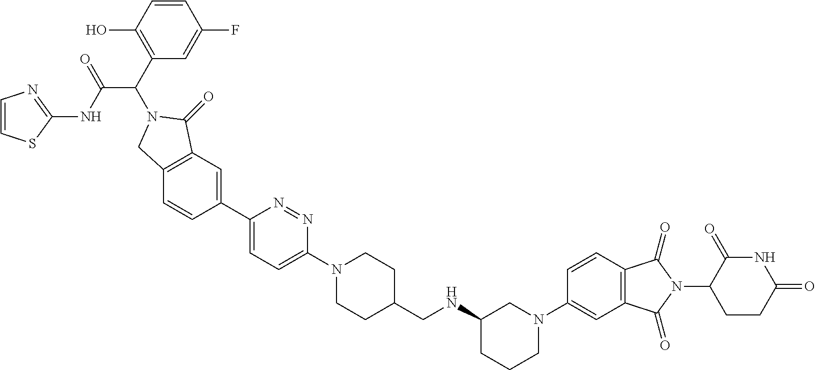 [SMILES: O=C1CCC(N2C(=O)c3ccc(N4CCC[C@@H](NCC5CCN(c6ccc(-c7ccc8c(c7)C(=O)N(C(C(=O)Nc7nccs7)c7cc(F)ccc7O)C8)nn6)CC5)C4)cc3C2=O)C(=O)N1] | 2-(6-(6-(4-((((3R)-1-(2-(2,6-dioxopiperidin-3- yl)-1,3-dioxoisoindolin-5-yl)piperidin-3- yl)amino)methyl)piperidin-1-yl)pyridazin-3- yl)-1-oxoisoindolin-2-yl)-2-(5-fluoro-2- hydroxyphenyl)-N-(thiazol-2-yl)acetamide |
| | 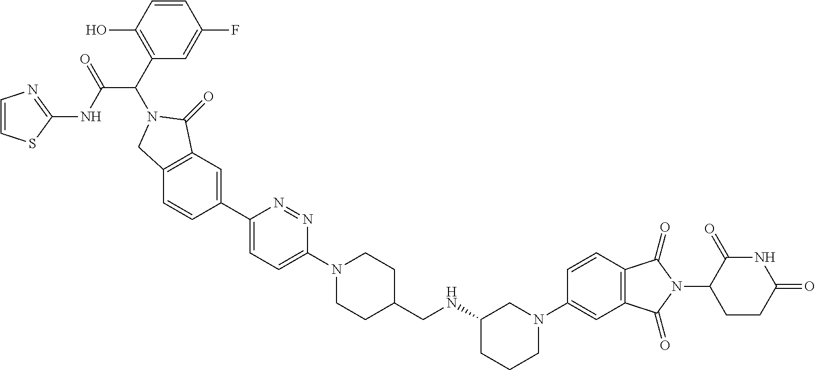 [SMILES: O=C1CCC(N2C(=O)c3ccc(N4CCC[C@H](NCC5CCN(c6ccc(-c7ccc8c(c7)C(=O)N(C(C(=O)Nc7nccs7)c7cc(F)ccc7O)C8)nn6)CC5)C4)cc3C2=O)C(=O)N1] | 2-(6-(6-(4-((((3S)-1-(2-(2,6-dioxopiperidin-3- yl)-1,3-dioxoisoindolin-5-yl)piperidin-3- yl)amino)methyl)piperidin-1-yl)pyridazin-3- yl)-1-oxoisoindolin-2-yl)-2-(5-fluoro-2- hydroxyphenyl)-N-(thiazol-2-yl)acetamide |
| | 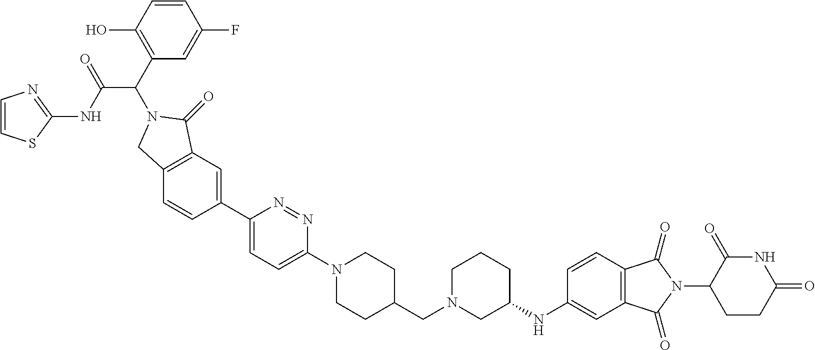 [SMILES: O=C1CCC(N2C(=O)c3ccc(N[C@H]4CCCN(CC5CCN(c6ccc(-c7ccc8c(c7)C(=O)N(C(C(=O)Nc7nccs7)c7cc(F)ccc7O)C8)nn6)CC5)C4)cc3C2=O)C(=O)N1] | 2-(6-(6-(4-(((3S)-3-((2-(2,6-dioxopiperidin-3- yl)-1,3-dioxoisoindolin-5-yl)amino)piperidin-1- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 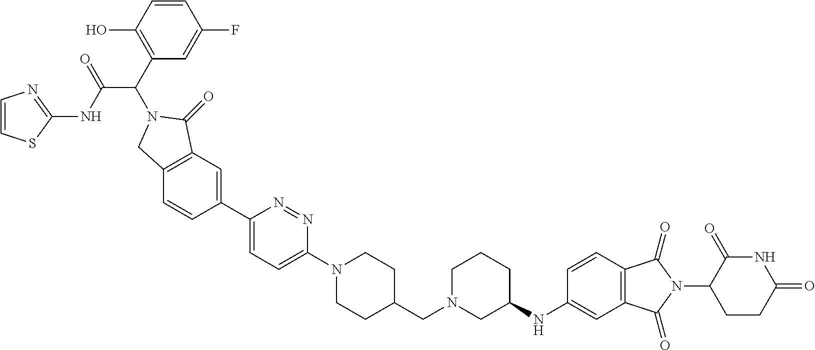 [SMILES: O=C1CCC(N2C(=O)c3ccc(N[C@@H]4CCCN(CC5CCN(c6ccc(-c7ccc8c(c7)C(=O)N(C(C(=O)Nc7nccs7)c7cc(F)ccc7O)C8)nn6)CC5)C4)cc3C2=O)C(=O)N1] | 2-(6-(6-(4-(((3R)-3-((2-(2,6-dioxopiperidin-3- yl)-1,3-dioxoisoindolin-5-yl)amino)piperidin-1- yl)methyl)piperidin-1-yl)pyridazin-3-yl)-1- oxoisoindolin-2-yl)-2-(5-fluoro-2- hydroxyphenyl)-N-(thiazol-2-yl)acetamide |
| | 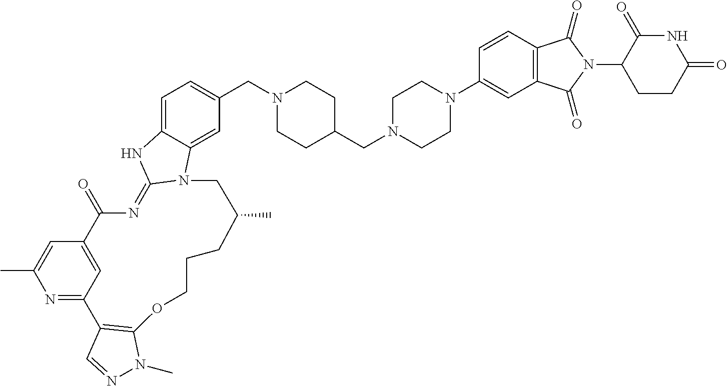 [SMILES: Cc1cc2cc(n1)-c1cnn(C)c1OCCC[C@@H](C)CN1/C(=N/C2=O)Nc2ccc(CN3CCC(CN4CCN(c5ccc6c(c5)C(=O)N(C5CCC(=O)NC5=O)C6=O)CC4)CC3)cc21] | 2-(2,6-dioxopiperidin-3-yl)-5-(4-((1-(((R,E)- 1¹,2⁶,7-trimethyl-3-oxo-5²,5³-dihydro-1¹H,5¹- 11-oxa-4-aza-5(2,1)-benzo[d]imidazola-2(2,4)- pyridina-1(4,5)-pyrazolacycloundecaphane-5⁶- yl)methyl)piperidin-4-yl)methyl)piperazin-1- yl)isoindoline-1,3-dione |
| | 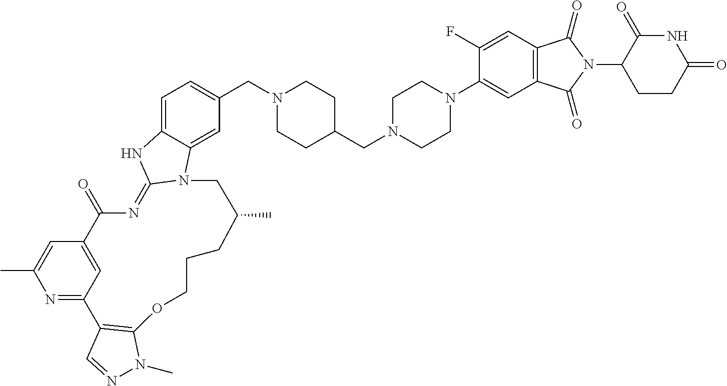 [SMILES: Cc1cc2cc(n1)-c1cnn(C)c1OCCC[C@@H](C)CN1/C(=N/C2=O)Nc2ccc(CN3CCC(CN4CCN(c5cc6c(cc5F)C(=O)N(C5CCC(=O)NC5=O)C6=O)CC4)CC3)cc21] | 2-(2,6-dioxopiperidin-3-yl)-5-fluoro-6-(4-((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)piperazin-1- yl)isoindoline-1,3-dione |
| | 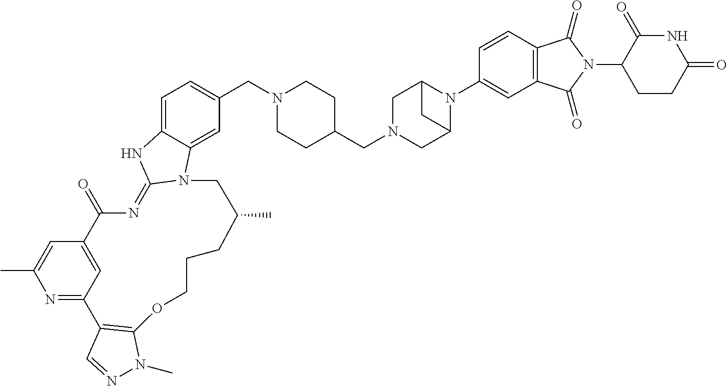 [SMILES: Cc1cc2cc(n1)-c1cnn(C)c1OCCC[C@@H](C)CN1/C(=N/C2=O)Nc2ccc(CN3CCC(CN4CC5CC(C4)N5c4ccc5c(c4)C(=O)N(C4CCC(=O)NC4=O)C5=O)CC3)cc21] | 2-(2,6-dioxopiperidin-3-yl)-5-(3-((1-(((R,E)- 1¹,2⁶,7-trimethyl-3-oxo-5²,5³-dihydro-1¹H,5¹- 11-oxa-4-aza-5(2,1)-benzo[d]imidazola-2(2,4)- pyridina-1(4,5)-pyrazolacycloundecaphane-5⁶- yl)methyl)piperidin-4-yl)methyl)-3,6- diazabicyclo[3.1.1]heptan-6-yl)isoindoline-1,3- dione |
| | 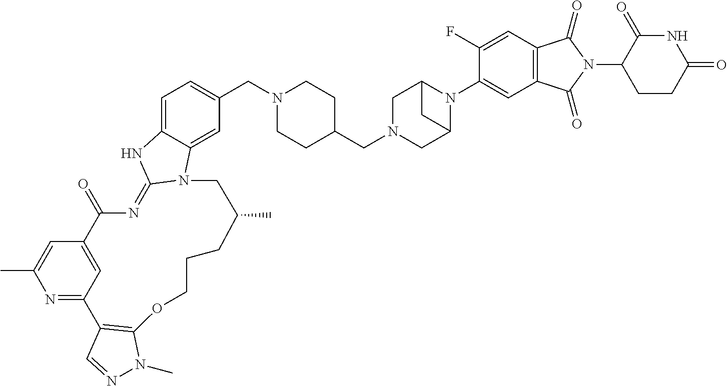 [SMILES: Cc1cc2cc(n1)-c1cnn(C)c1OCCC[C@@H](C)CN1/C(=N/C2=O)Nc2ccc(CN3CCC(CN4CC5CC(C4)N5c4cc5c(cc4F)C(=O)N(C4CCC(=O)NC4=O)C5=O)CC3)cc21] | 2-(2,6-dioxopiperidin-3-yl)-5-fluoro-6-(3-((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)-3,6- diazabicyclo[3.1.1]heptan-6-yl)isoindoline-1,3- dione |
| | 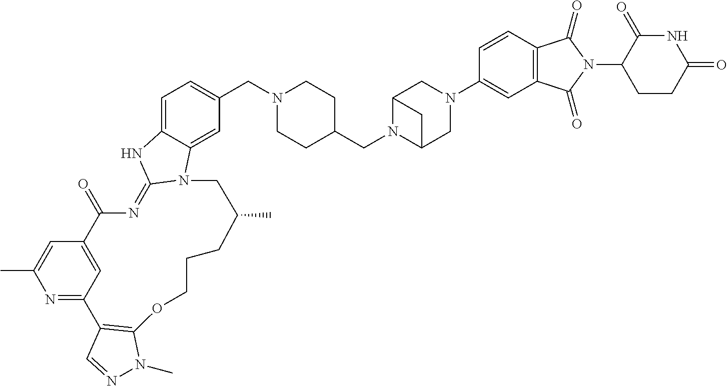 [SMILES: Cc1cc2cc(n1)-c1cnn(C)c1OCCC[C@@H](C)CN1/C(=N/C2=O)Nc2ccc(CN3CCC(CN4C5CC4CN(c4ccc6c(c4)C(=O)N(C4CCC(=O)NC4=O)C6=O)C5)CC3)cc21] | 2-(2,6-dioxopiperidin-3-yl)-5-(6-((1-(((R,E)- 1¹,2⁶,7-trimethyl-3-oxo-5²,5³-dihydro-1¹H,5¹- 11-oxa-4-aza-5(2,1)-benzo[d]imidazola-2(2,4)- pyridina-1(4,5)-pyrazolacycloundecaphane-5⁶- yl)methyl)piperidin-4-yl)methyl)-3,6- diazabicyclo[3.1.1]heptan-3-yl)isoindoline-1,3- dione |
| | 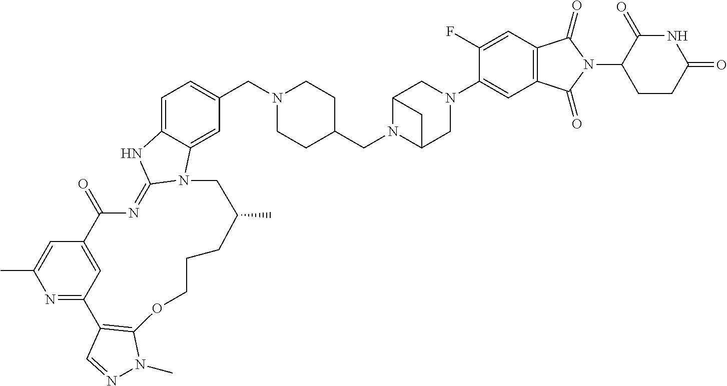 [SMILES: Cc1cc2cc(n1)-c1cnn(C)c1OCCC[C@@H](C)CN1/C(=N/C2=O)Nc2ccc(CN3CCC(CN4C5CC4CN(c4cc6c(cc4F)C(=O)N(C4CCC(=O)NC4=O)C6=O)C5)CC3)cc21] | 2-(2,6-dioxopiperidin-3-yl)-5-fluoro-6-(6-((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)-3,6- diazabicyclo[3.1.1]heptan-3-yl)isoindoline-1,3- dione |
| | 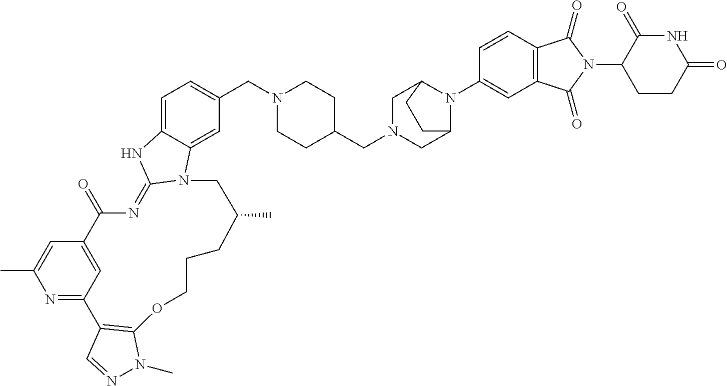 [SMILES: Cc1cc2cc(n1)-c1cnn(C)c1OCCC[C@@H](C)CN1/C(=N/C2=O)Nc2ccc(CN3CCC(CN4CC5CCC(C4)N5c4ccc5c(c4)C(=O)N(C4CCC(=O)NC4=O)C5=O)CC3)cc21] | 2-(2,6-dioxopiperidin-3-yl)-5-(3-((1-(((R,E)- 1¹,2⁶,7-trimethyl-3-oxo-5²,5³-dihydro-1¹H,5¹- 11-oxa-4-aza-5(2,1)-benzo[d]imidazola-2(2,4)- pyridina-1(4,5)-pyrazolacycloundecaphane-5⁶- yl)methyl)piperidin-4-yl)methyl)-3,8- diazabicyclo[3.2.1]octan-8-yl)isoindoline-1,3- dione |
| | 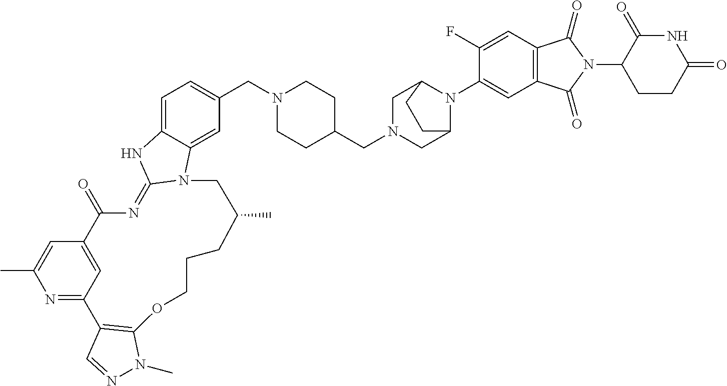 [SMILES: Cc1cc2cc(n1)-c1cnn(C)c1OCCC[C@@H](C)CN1/C(=N/C2=O)Nc2ccc(CN3CCC(CN4CC5CCC(C4)N5c4cc5c(cc4F)C(=O)N(C4CCC(=O)NC4=O)C5=O)CC3)cc21] | 2-(2,6-dioxopiperidin-3-yl)-5-fluoro-6-(3-((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)-3,8- diazabicyclo[3.2.1]octan-8-yl)isoindoline-1,3- dione |
| | 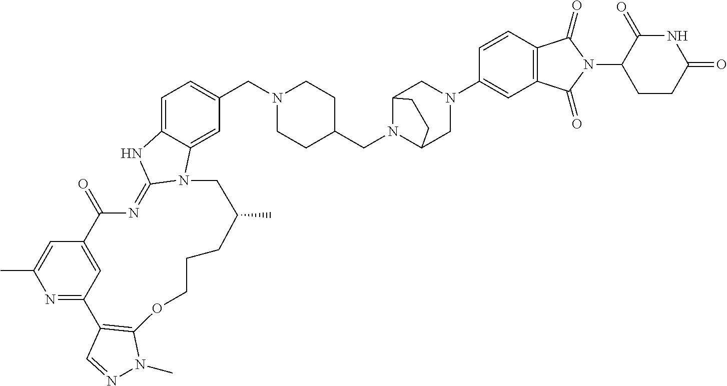 [SMILES: Cc1cc2cc(n1)-c1cnn(C)c1OCCC[C@@H](C)CN1/C(=N/C2=O)Nc2ccc(CN3CCC(CN4C5CCC4CN(c4ccc6c(c4)C(=O)N(C4CCC(=O)NC4=O)C6=O)C5)CC3)cc21] | 2-(2,6-dioxopiperidin-3-yl)-5-(8-((1-(((R,E)- 1¹,2⁶,7-trimethyl-3-oxo-5²,5³-dihydro-1¹H,5¹- 11-oxa-4-aza-5(2,1)-benzo[d]imidazola-2(2,4)- pyridina-1(4,5)-pyrazolacycloundecaphane-5⁶- yl)methyl)piperidin-4-yl)methyl)-3,8- diazabicyclo[3.2.1]octan-3-yl)isoindoline-1,3- dione |
| | 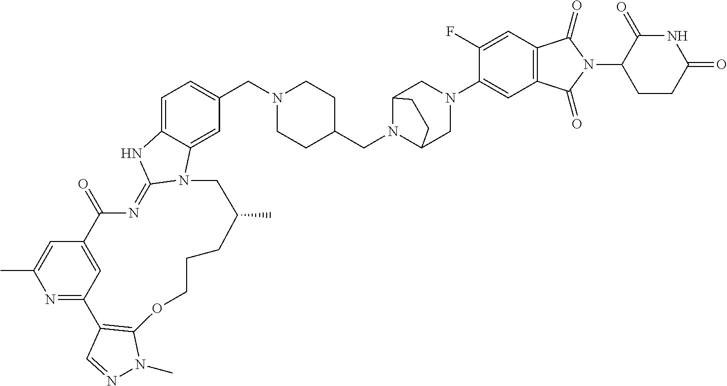 [SMILES: Cc1cc2cc(n1)-c1cnn(C)c1OCCC[C@@H](C)CN1/C(=N/C2=O)Nc2ccc(CN3CCC(CN4C5CCC4CN(c4cc6c(cc4F)C(=O)N(C4CCC(=O)NC4=O)C6=O)C5)CC3)cc21] | 2-(2,6-dioxopiperidin-3-yl)-5-fluoro-6-(8-((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)-3,8- diazabicyclo[3.2.1]octan-3-yl)isoindoline-1,3- dione |
| | 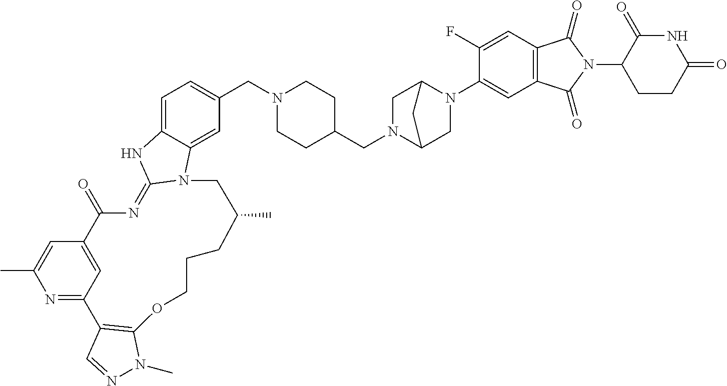 [SMILES: Cc1cc2cc(n1)-c1cnn(C)c1OCCC[C@@H](C)CN1/C(=N/C2=O)Nc2ccc(CN3CCC(CN4CC5CC4CN5c4cc5c(cc4F)C(=O)N(C4CCC(=O)NC4=O)C5=O)CC3)cc21] | 2-(2,6-dioxopiperidin-3-yl)-5-fluoro-6-(5-((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)-2,5- diazabicyclo[2.2.1]heptan-2-yl)isoindoline-1,3- dione |
| | 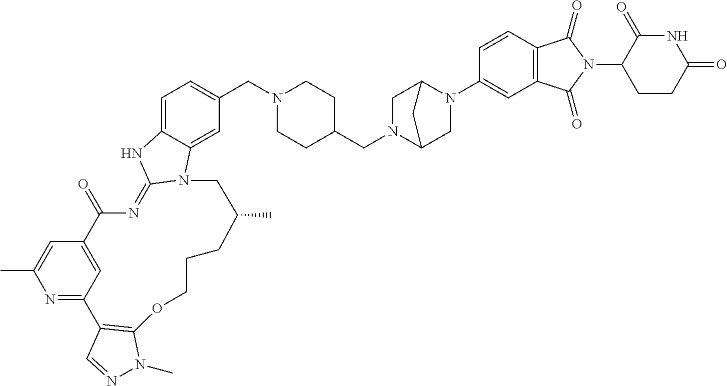 [SMILES: Cc1cc2cc(n1)-c1cnn(C)c1OCCC[C@@H](C)CN1/C(=N/C2=O)Nc2ccc(CN3CCC(CN4CC5CC4CN5c4ccc5c(c4)C(=O)N(C4CCC(=O)NC4=O)C5=O)CC3)cc21] | 2-(2,6-dioxopiperidin-3-yl)-5-(5-((1-(((R,E)- 1¹,2⁶,7-trimethyl-3-oxo-5²,5³-dihydro-1¹H,5¹- 11-oxa-4-aza-5(2,1)-benzo[d]imidazola-2(2,4)- pyridina-1(4,5)-pyrazolacycloundecaphane-5⁶- yl)methyl)piperidin-4-yl)methyl)-2,5- diazabicyclo[2.2.1]heptan-2-yl)isoindoline-1,3- dione |
| | 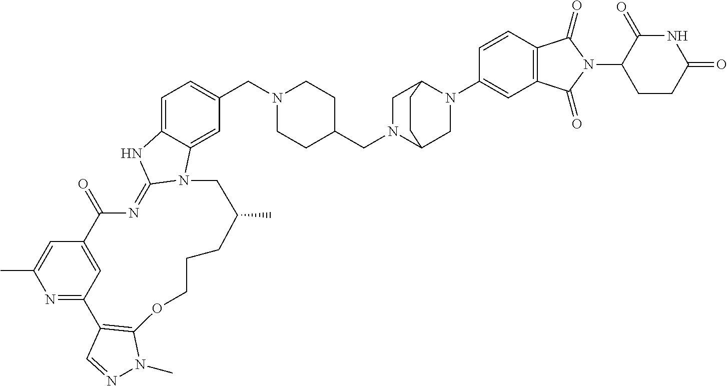 [SMILES: Cc1cc2cc(n1)-c1cnn(C)c1OCCC[C@@H](C)CN1/C(=N/C2=O)Nc2ccc(CN3CCC(CN4CC5CCC4CN5c4ccc5c(c4)C(=O)N(C4CCC(=O)NC4=O)C5=O)CC3)cc21] | 2-(2,6-dioxopiperidin-3-yl)-5-(5-((1-(((R,E)- 1¹,2⁶,7-trimethyl-3-oxo-5²,5³-dihydro-1¹H,5¹- 11-oxa-4-aza-5(2,1)-benzo[d]imidazola-2(2,4)- pyridina-1(4,5)-pyrazolacycloundecaphane-5⁶- yl)methyl)piperidin-4-yl)methyl)-2,5- diazabicyclo[2.2.2]octan-2-yl)isoindoline-1,3- dione |
| | 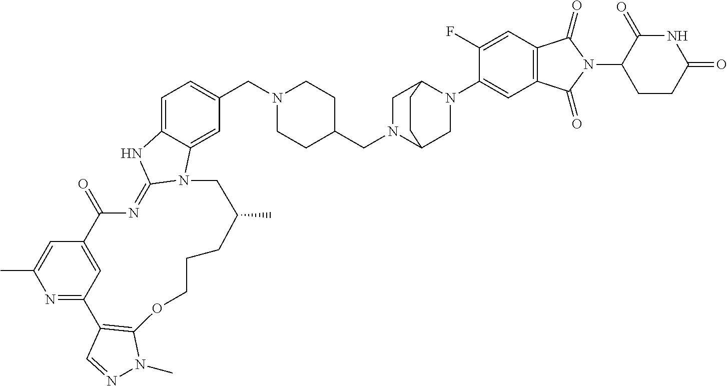 [SMILES: Cc1cc2cc(n1)-c1cnn(C)c1OCCC[C@@H](C)CN1/C(=N/C2=O)Nc2ccc(CN3CCC(CN4CC5CCC4CN5c4cc5c(cc4F)C(=O)N(C4CCC(=O)NC4=O)C5=O)CC3)cc21] | 2-(2,6-dioxopiperidin-3-yl)-5-fluoro-6-(5-((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)-2,5- diazabicyclo[2.2.2]octan-2-yl)isoindoline-1,3- dione |
| | 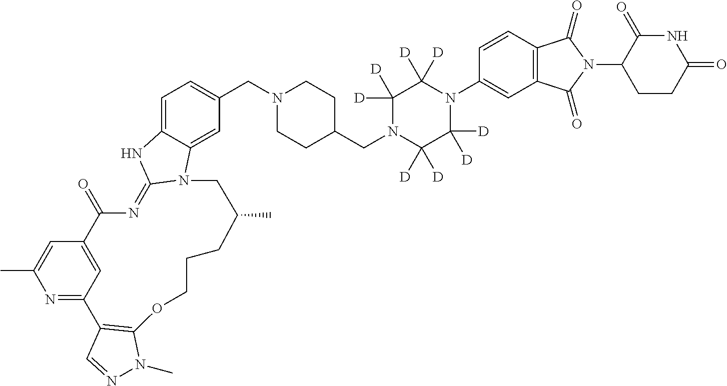 [SMILES: [2H]C1([2H])N(CC2CCN(Cc3ccc4c(c3)N3C[C@H](C)CCCOc5c(cnn5C)-c5cc(cc(C)n5)C(=O)/N=C/3N4)CC2)C([2H])([2H])C([2H])([2H])N(c2ccc3c(c2)C(=O)N(C2CCC(=O)NC2=O)C3=O)C1([2H])[2H]] | 2-(2,6-dioxopiperidin-3-yl)-5-(4-((1-(((R,E)- 1¹,2⁶,7-trimethyl-3-oxo-5²,5³-dihydro-1¹H,5¹- 11-oxa-4-aza-5(2,1)-benzo[d]imidazola-2(2,4)- pyridina-1(4,5)-pyrazolacycloundecaphane-5⁶- yl)methyl)piperidin-4-yl)methyl)piperazin-1-yl- 2,2,3,3,5,5,6,6-d₈)isoindoline-1,3-dione |
| | 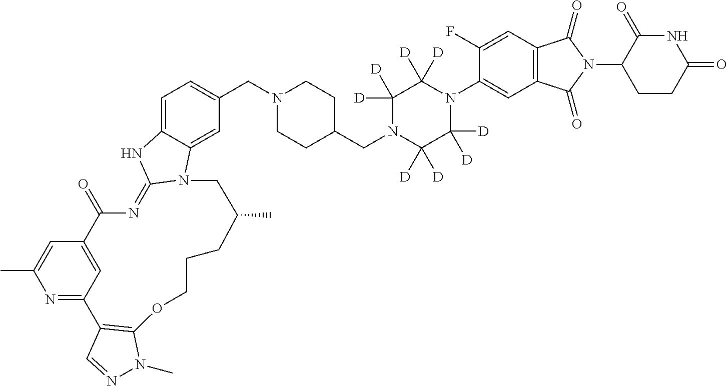 [SMILES: [2H]C1([2H])N(CC2CCN(Cc3ccc4c(c3)N3C[C@H](C)CCCOc5c(cnn5C)-c5cc(cc(C)n5)C(=O)/N=C/3N4)CC2)C([2H])([2H])C([2H])([2H])N(c2cc3c(cc2F)C(=O)N(C2CCC(=O)NC2=O)C3=O)C1([2H])[2H]] | 2-(2,6-dioxopiperidin-3-yl)-5-fluoro-6-(4-((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)piperazin-1-yl- 2,2,3,3,5,5,6,6-d₈)isoindoline-1,3-dione |
| | 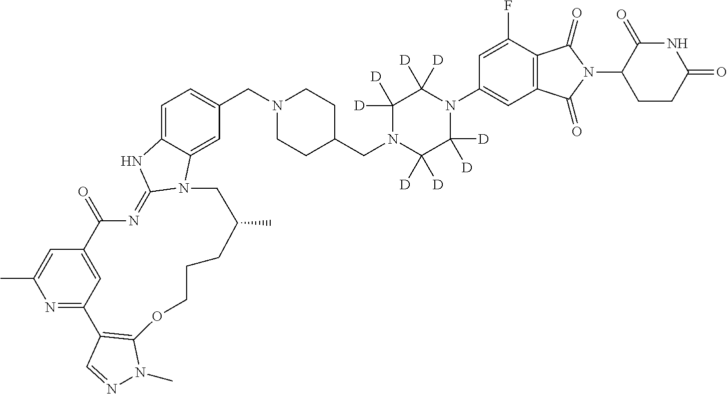 [SMILES: [2H]C1([2H])N(CC2CCN(Cc3ccc4c(c3)N3C[C@H](C)CCCOc5c(cnn5C)-c5cc(cc(C)n5)C(=O)/N=C/3N4)CC2)C([2H])([2H])C([2H])([2H])N(c2cc(F)c3c(c2)C(=O)N(C2CCC(=O)NC2=O)C3=O)C1([2H])[2H]] | 2-(2,6-dioxopiperidin-3-yl)-4-fluoro-6-(4-((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)piperazin-1-yl- 2,2,3,3,5,5,6,6-d₈)isoindoline-1,3-dione |
| | 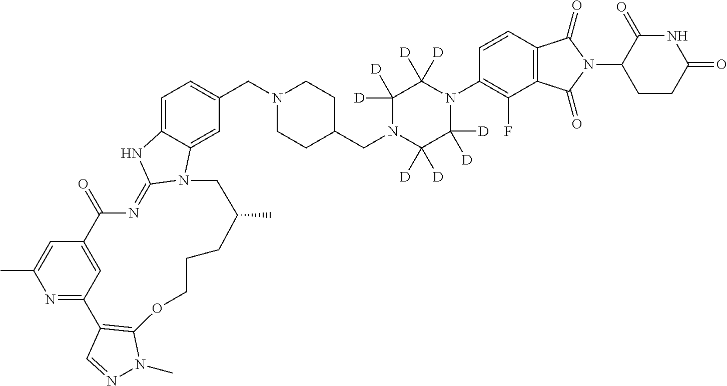 [SMILES: [2H]C1([2H])N(CC2CCN(Cc3ccc4c(c3)N3C[C@H](C)CCCOc5c(cnn5C)-c5cc(cc(C)n5)C(=O)/N=C/3N4)CC2)C([2H])([2H])C([2H])([2H])N(c2ccc3c(c2F)C(=O)N(C2CCC(=O)NC2=O)C3=O)C1([2H])[2H]] | 2-(2,6-dioxopiperidin-3-yl)-4-fluoro-5-(4-((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)piperazin-1-yl- 2,2,3,3,5,5,6,6-d₈)isoindoline-1,3-dione |
| | 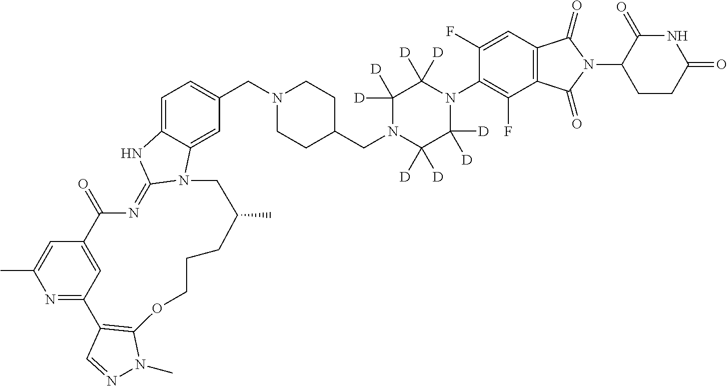 [SMILES: [2H]C1([2H])N(CC2CCN(Cc3ccc4c(c3)N3C[C@H](C)CCCOc5c(cnn5C)-c5cc(cc(C)n5)C(=O)/N=C/3N4)CC2)C([2H])([2H])C([2H])([2H])N(c2c(F)cc3c(c2F)C(=O)N(C2CCC(=O)NC2=O)C3=O)C1([2H])[2H]] | 2-(2,6-dioxopiperidin-3-yl)-4,6-difluoro-5-(4- ((1-(((R,E)-1¹,2⁶,7-trimethyl-3-oxo-5²,5³- dihydro-1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)piperazin-1-yl- 2,2,3,3,5,5,6,6-d₈)isoindoline-1,3-dione |
| | 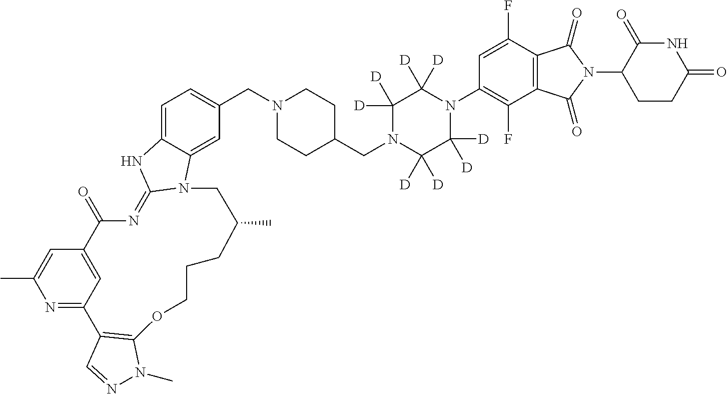 [SMILES: [2H]C1([2H])N(CC2CCN(Cc3ccc4c(c3)N3C[C@H](C)CCCOc5c(cnn5C)-c5cc(cc(C)n5)C(=O)/N=C/3N4)CC2)C([2H])([2H])C([2H])([2H])N(c2cc(F)c3c(c2F)C(=O)N(C2CCC(=O)NC2=O)C3=O)C1([2H])[2H]] | 2-(2,6-dioxopiperidin-3-yl)-4,7-difluoro-5-(4- ((1-(((R,E)-1¹,2⁶,7-trimethyl-3-oxo-5²,5³- dihydro-1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)piperazin-1-yl- 2,2,3,3,5,5,6,6-d₈)isoindoline-1,3-dione |
| | 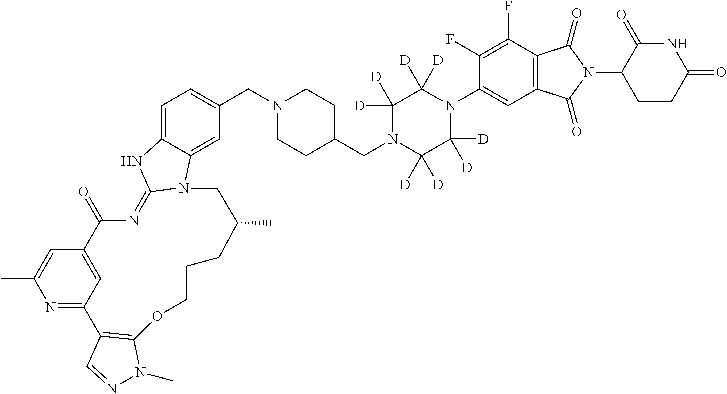 [SMILES: [2H]C1([2H])N(CC2CCN(Cc3ccc4c(c3)N3C[C@H](C)CCCOc5c(cnn5C)-c5cc(cc(C)n5)C(=O)/N=C/3N4)CC2)C([2H])([2H])C([2H])([2H])N(c2cc3c(c(F)c2F)C(=O)N(C2CCC(=O)NC2=O)C3=O)C1([2H])[2H]] | 2-(2,6-dioxopiperidin-3-yl)-4,5-difluoro-6-(4- ((1-(((R,E)-1¹,2⁶,7-trimethyl-3-oxo-5²,5³- dihydro-1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)piperazin-1-yl- 2,2,3,3,5,5,6,6-d₈)isoindoline-1,3-dione |
| | 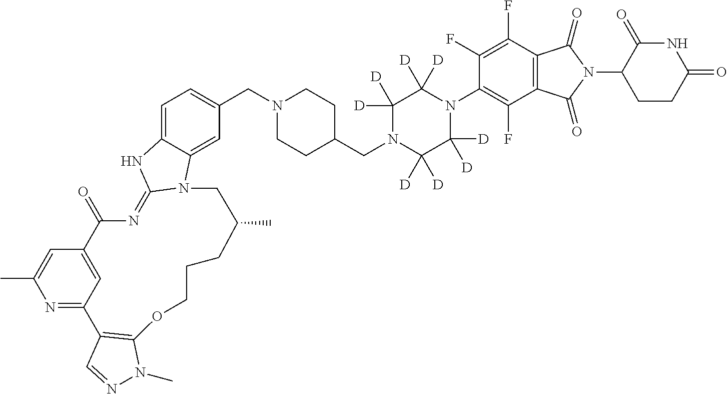 [SMILES: [2H]C1([2H])N(CC2CCN(Cc3ccc4c(c3)N3C[C@H](C)CCCOc5c(cnn5C)-c5cc(cc(C)n5)C(=O)/N=C/3N4)CC2)C([2H])([2H])C([2H])([2H])N(c2c(F)c(F)c3c(c2F)C(=O)N(C2CCC(=O)NC2=O)C3=O)C1([2H])[2H]] | 2-(2,6-dioxopiperidin-3-yl)-4,5,7-trifluoro-6- (4-((1-(((R,E)-1¹,2⁶,7-trimethyl-3-oxo-5²,5³- dihydro-1¹H,5¹-11-oxa-4-aza-5(2,1)- benzo[d]imidazola-2(2,4)-pyridina-1(4,5)- pyrazolacycloundecaphane-56 yl)methyl)piperidin-4-yl)methyl)piperazin-1-yl- 2,2,3,3,5,5,6,6-d₈)isoindoline-1,3-dione |
| | 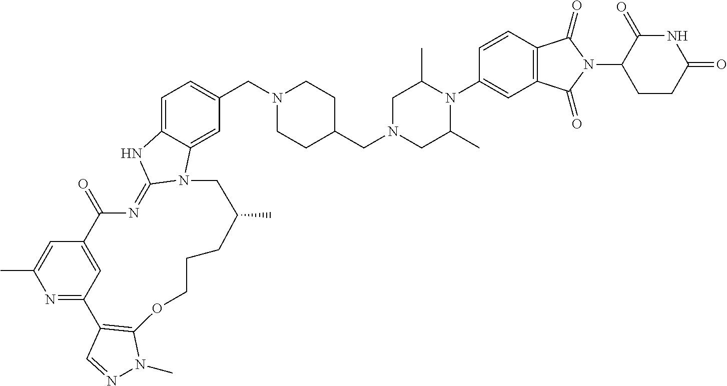 [SMILES: Cc1cc2cc(n1)-c1cnn(C)c1OCCC[C@@H](C)CN1/C(=N/C2=O)Nc2ccc(CN3CCC(CN4CC(C)N(c5ccc6c(c5)C(=O)N(C5CCC(=O)NC5=O)C6=O)C(C)C4)CC3)cc21] | 5-(2,6-dimethyl-4-((1-(((R,E)-1¹,2⁶,7-trimethyl- 3-oxo-5²,5³-dihydro-1¹H,5¹-11-oxa-4-aza- 5(2,1)-benzo[d]imidazola-2(2,4)-pyridina- 1(4,5)-pyrazolacycloundecaphane-5⁶- yl)methyl)piperidin-4-yl)methyl)piperazin-1- yl)-2-(2,6-dioxopiperidin-3-yl)isoindoline-1,3- dione |
| | 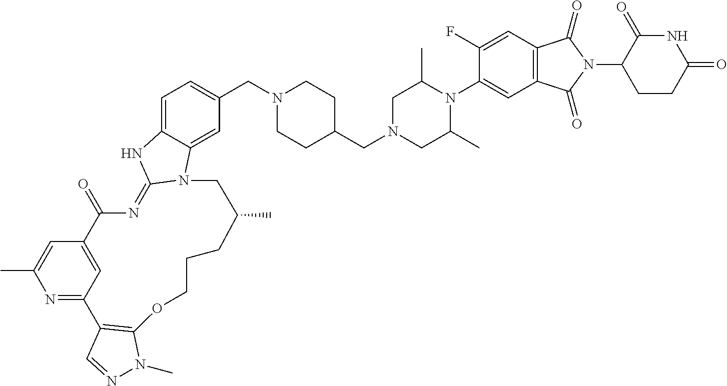 [SMILES: Cc1cc2cc(n1)-c1cnn(C)c1OCCC[C@@H](C)CN1/C(=N/C2=O)Nc2ccc(CN3CCC(CN4CC(C)N(c5cc6c(cc5F)C(=O)N(C5CCC(=O)NC5=O)C6=O)C(C)C4)CC3)cc21] | 5-(2,6-dimethyl-4-((1-(((R,E)-1¹,2⁶,7-trimethyl- 3-oxo-5²,5³-dihydro-1¹H,5¹-11-oxa-4-aza- 5(2,1)-benzo[d]imidazola-2(2,4)-pyridina- 1(4,5)-pyrazolacycloundecaphane-5⁶- yl)methyl)piperidin-4-yl)methyl)piperazin-1- yl)-2-(2,6-dioxopiperidin-3-yl)-6- fluoroisoindoline-1,3-dione |
| | 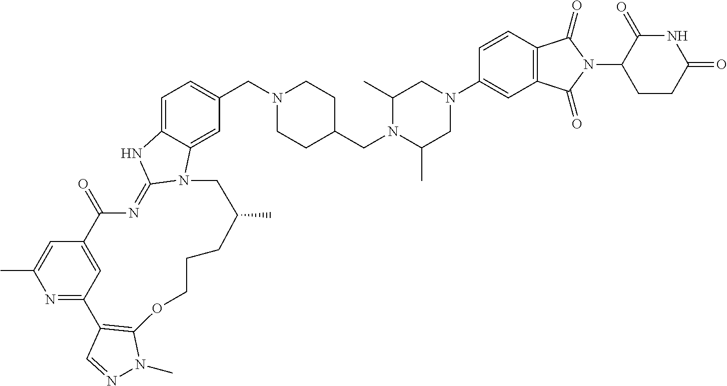 [SMILES: Cc1cc2cc(n1)-c1cnn(C)c1OCCC[C@@H](C)CN1/C(=N/C2=O)Nc2ccc(CN3CCC(CN4C(C)CN(c5ccc6c(c5)C(=O)N(C5CCC(=O)NC5=O)C6=O)CC4C)CC3)cc21] | 5-(3,5-dimethyl-4-((1-(((R,E)-1¹,2⁶,7-trimethyl- 3-oxo-5²,5³-dihydro-1¹H,5¹-11-oxa-4-aza- 5(2,1)-benzo[d]imidazola-2(2,4)-pyridina- 1(4,5)-pyrazolacycloundecaphane-5⁶- yl)methyl)piperidin-4-yl)methyl)piperazin-1- yl)-2-(2,6-dioxopiperidin-3-yl)isoindoline-1,3- dione |
| | 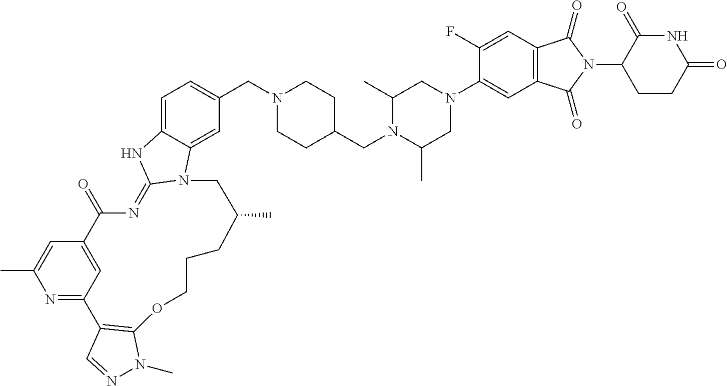 [SMILES: Cc1cc2cc(n1)-c1cnn(C)c1OCCC[C@@H](C)CN1/C(=N/C2=O)Nc2ccc(CN3CCC(CN4C(C)CN(c5cc6c(cc5F)C(=O)N(C5CCC(=O)NC5=O)C6=O)CC4C)CC3)cc21] | 5-(3,5-dimethyl-4-((1-(((R,E)-1¹,2⁶,7-trimethyl- 3-oxo-5²,5³-dihydro-1¹H,5¹-11-oxa-4-aza- 5(2,1)-benzo[d]imidazola-2(2,4)-pyridina- 1(4,5)-pyrazolacycloundecaphane-5⁶- yl)methyl)piperidin-4-yl)methyl)piperazin-1- yl)-2-(2,6-dioxopiperidin-3-yl)-6- fluoroisoindoline-1,3-dione |
| | 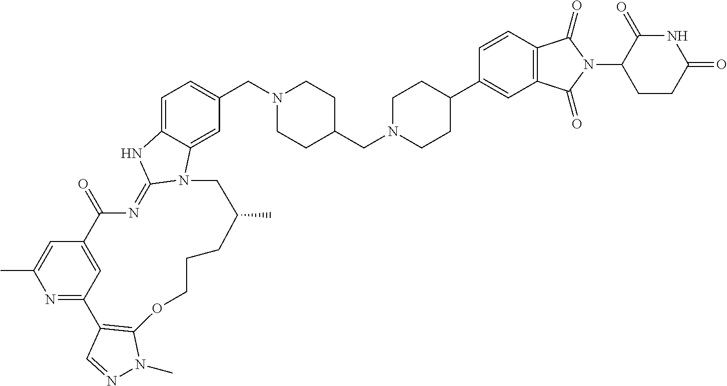 [SMILES: Cc1cc2cc(n1)-c1cnn(C)c1OCCC[C@@H](C)CN1/C(=N/C2=O)Nc2ccc(CN3CCC(CN4CCC(c5ccc6c(c5)C(=O)N(C5CCC(=O)NC5=O)C6=O)CC4)CC3)cc21] | 2-(2,6-dioxopiperidin-3-yl)-5-(1-((1-(((R,E)- 1¹,2⁶,7-trimethyl-3-oxo-5²,5³-dihydro-1¹H,5¹- 11-oxa-4-aza-5(2,1)-benzo[d]imidazola-2(2,4)- pyridina-1(4,5)-pyrazolacycloundecaphane-5⁶- yl)methyl)piperidin-4-yl)methyl)piperidin-4- yl)isoindoline-1,3-dione |
| | 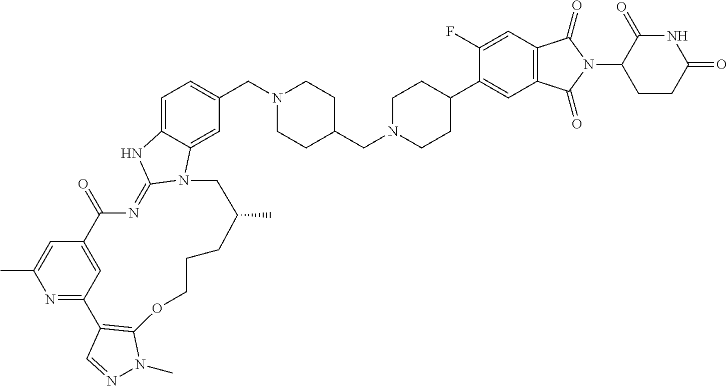 [SMILES: Cc1cc2cc(n1)-c1cnn(C)c1OCCC[C@@H](C)CN1/C(=N/C2=O)Nc2ccc(CN3CCC(CN4CCC(c5cc6c(cc5F)C(=O)N(C5CCC(=O)NC5=O)C6=O)CC4)CC3)cc21] | 2-(2,6-dioxopiperidin-3-yl)-5-fluoro-6-(1-((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)piperidin-4- yl)isoindoline-1,3-dione |
| | 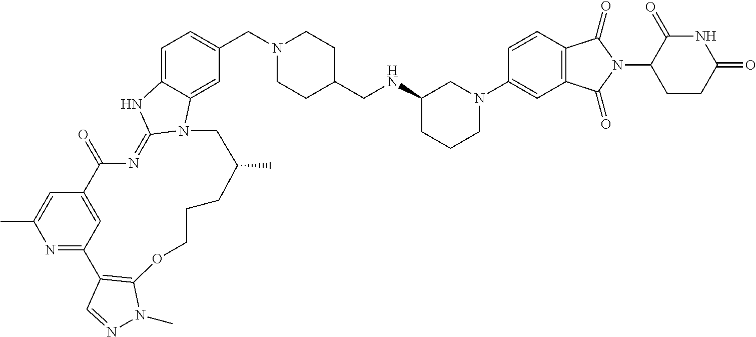 [SMILES: Cc1cc2cc(n1)-c1cnn(C)c1OCCC[C@@H](C)CN1/C(=N/C2=O)Nc2ccc(CN3CCC(CN[C@@H]4CCCN(c5ccc6c(c5)C(=O)N(C5CCC(=O)NC5=O)C6=O)C4)CC3)cc21] | 2-(2,6-dioxopiperidin-3-yl)-5-((R)-3-(((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-56 yl)methyl)piperidin-4- yl)methyl)amino)piperidin-1-yl)isoindoline- 1,3-dione |
| | 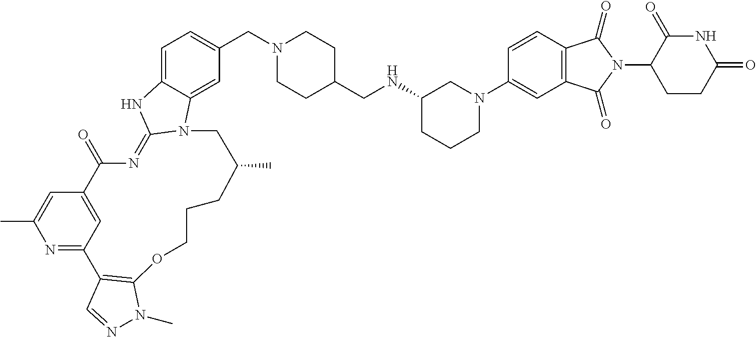 [SMILES: Cc1cc2cc(n1)-c1cnn(C)c1OCCC[C@@H](C)CN1/C(=N/C2=O)Nc2ccc(CN3CCC(CN[C@H]4CCCN(c5ccc6c(c5)C(=O)N(C5CCC(=O)NC5=O)C6=O)C4)CC3)cc21] | 2-(2,6-dioxopiperidin-3-yl)-5-((S)-3-(((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4- yl)methyl)amino)piperidin-1-yl)isoindoline- 1,3-dione |
| | 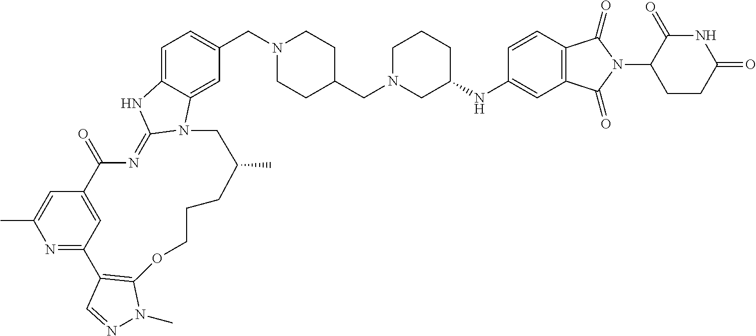 [SMILES: Cc1cc2cc(n1)-c1cnn(C)c1OCCC[C@@H](C)CN1/C(=N/C2=O)Nc2ccc(CN3CCC(CN4CCC[C@H](Nc5ccc6c(c5)C(=O)N(C5CCC(=O)NC5=O)C6=O)C4)CC3)cc21] | 2-(2,6-dioxopiperidin-3-yl)-5-(((S)-1-((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)piperidin-3- yl)amino)isoindoline-1,3-dione |
| | 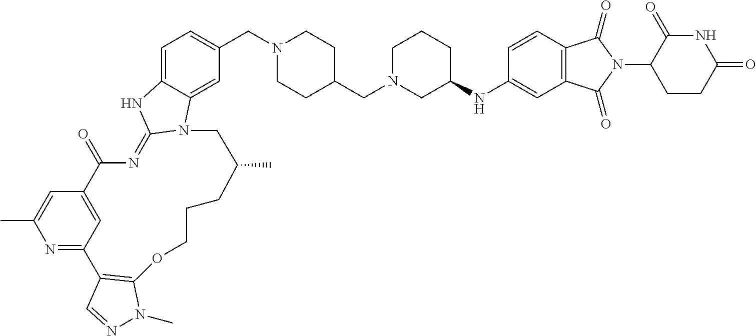 [SMILES: Cc1cc2cc(n1)-c1cnn(C)c1OCCC[C@@H](C)CN1/C(=N/C2=O)Nc2ccc(CN3CCC(CN4CCC[C@@H](Nc5ccc6c(c5)C(=O)N(C5CCC(=O)NC5=O)C6=O)C4)CC3)cc21] | 2-(2,6-dioxopiperidin-3-yl)-5-(((R)-1-((1- (((R,E)-1¹,2⁶,7-trimethyl-3-oxo-5²,5³-dihydro- 1¹H,5¹-11-oxa-4-aza-5(2,1)- benzo[d]imidazola-2(2,4)-pyridina-1(4,5)- pyrazolacycloundecaphane-5⁶- yl)methyl)piperidin-4-yl)methyl)piperidin-3- yl)amino)isoindoline-1,3-dione |
| | 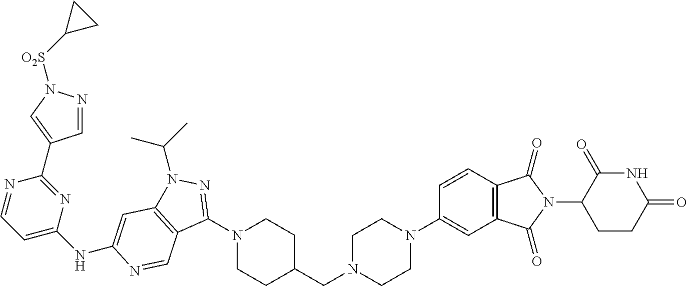 [SMILES: CC(C)n1nc(N2CCC(CN3CCN(c4ccc5c(c4)C(=O)N(C4CCC(=O)NC4=O)C5=O)CC3)CC2)c2cnc(Nc3ccnc(-c4cnn(S(=O)(=O)C5CC5)c4)n3)cc21] | 5-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)piperazin-1-yl)-2-(2,6- dioxopiperidin-3-yl)isoindoline-1,3-dione |
| | 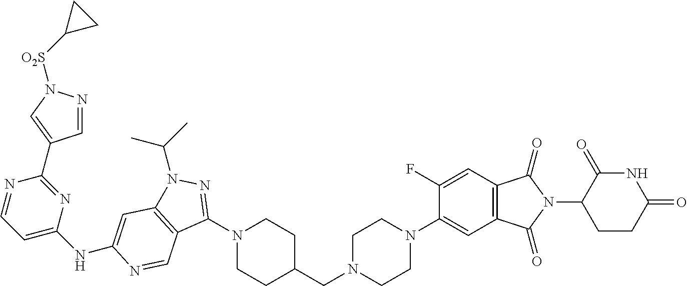 [SMILES: CC(C)n1nc(N2CCC(CN3CCN(c4cc5c(cc4F)C(=O)N(C4CCC(=O)NC4=O)C5=O)CC3)CC2)c2cnc(Nc3ccnc(-c4cnn(S(=O)(=O)C5CC5)c4)n3)cc21] | 5-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)piperazin-1-yl)-2-(2,6- dioxopiperidin-3-yl)-6-fluoroisoindoline-1,3- dione |
| | 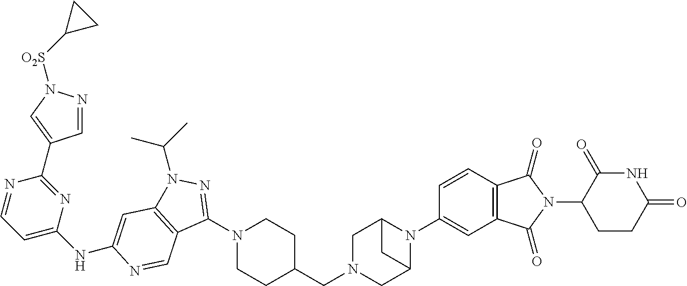 [SMILES: CC(C)n1nc(N2CCC(CN3CC4CC(C3)N4c3ccc4c(c3)C(=O)N(C3CCC(=O)NC3=O)C4=O)CC2)c2cnc(Nc3ccnc(-c4cnn(S(=O)(=O)C5CC5)c4)n3)cc21] | 5-(3-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)-3,6- diazabicyclo[3.1.1]heptan-6-yl)-2-(2,6- dioxopiperidin-3-yl)isoindoline-1,3-dione |
| | 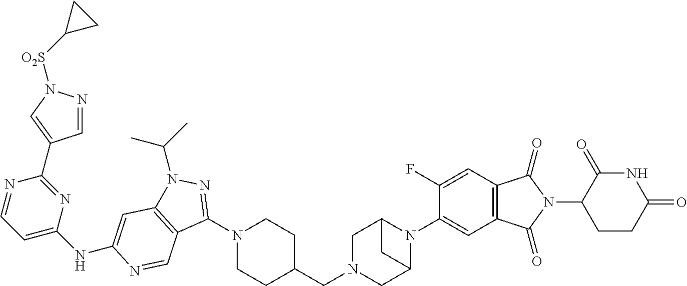 [SMILES: CC(C)n1nc(N2CCC(CN3CC4CC(C3)N4c3cc4c(cc3F)C(=O)N(C3CCC(=O)NC3=O)C4=O)CC2)c2cnc(Nc3ccnc(-c4cnn(S(=O)(=O)C5CC5)c4)n3)cc21] | 5-(3-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)-3,6- diazabicyclo[3.1.1]heptan-6-yl)-2-(2,6- dioxopiperidin-3-yl)-6-fluoroisoindoline-1,3- dione |
| | 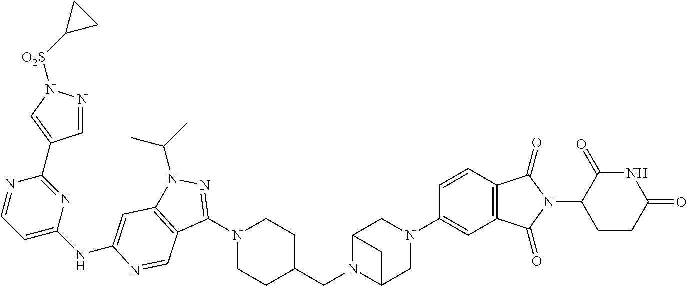 [SMILES: CC(C)n1nc(N2CCC(CN3C4CC3CN(c3ccc5c(c3)C(=O)N(C3CCC(=O)NC3=O)C5=O)C4)CC2)c2cnc(Nc3ccnc(-c4cnn(S(=O)(=O)C5CC5)c4)n3)cc21] | 5-(6-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)-3,6- diazabicyclo[3.1.1]heptan-3-yl)-2-(2,6- dioxopiperidin-3-yl)isoindoline-1,3-dione |
| | 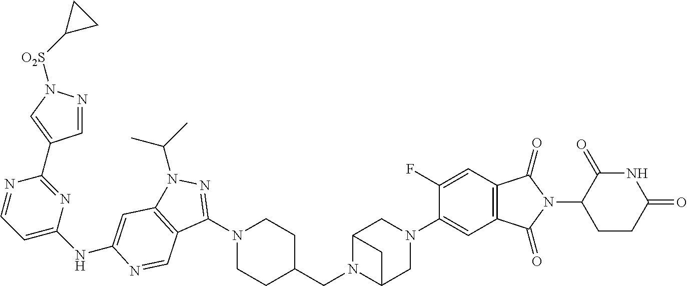 [SMILES: CC(C)n1nc(N2CCC(CN3C4CC3CN(c3cc5c(cc3F)C(=O)N(C3CCC(=O)NC3=O)C5=O)C4)CC2)c2cnc(Nc3ccnc(-c4cnn(S(=O)(=O)C5CC5)c4)n3)cc21] | 5-(6-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)-3,6- diazabicyclo[3.1.1]heptan-3-yl)-2-(2,6- dioxopiperidin-3-yl)-6-fluoroisoindoline-1,3- dione |
| | 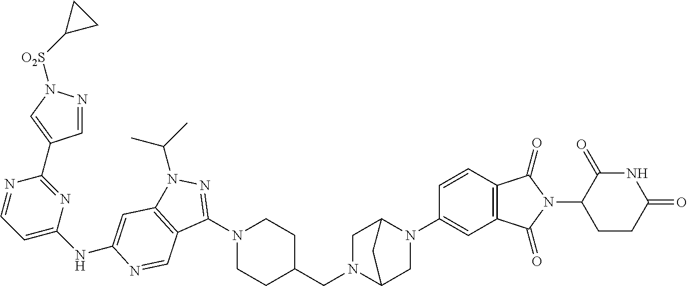 [SMILES: CC(C)n1nc(N2CCC(CN3CC4CC3CN4c3ccc4c(c3)C(=O)N(C3CCC(=O)NC3=O)C4=O)CC2)c2cnc(Nc3ccnc(-c4cnn(S(=O)(=O)C5CC5)c4)n3)cc21] | 5-(5-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)-2,5- diazabicyclo[2.2.1]heptan-2-yl)-2-(2,6- dioxopiperidin-3-yl)isoindoline-1,3-dione |
| | 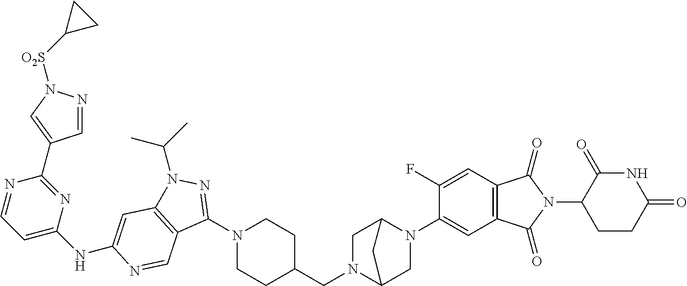 [SMILES: CC(C)n1nc(N2CCC(CN3CC4CC3CN4c3cc4c(cc3F)C(=O)N(C3CCC(=O)NC3=O)C4=O)CC2)c2cnc(Nc3ccnc(-c4cnn(S(=O)(=O)C5CC5)c4)n3)cc21] | 5-(5-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)-2,5- diazabicyclo[2.2.1]heptan-2-yl)-2-(2,6- dioxopiperidin-3-yl)-6-fluoroisoindoline-1,3- dione |
| | 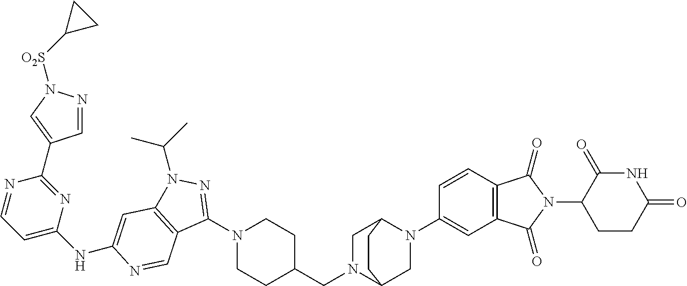 [SMILES: CC(C)n1nc(N2CCC(CN3CC4CCC3CN4c3ccc4c(c3)C(=O)N(C3CCC(=O)NC3=O)C4=O)CC2)c2cnc(Nc3ccnc(-c4cnn(S(=O)(=O)C5CC5)c4)n3)cc21] | 5-(5-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)-2,5- diazabicyclo[2.2.2]octan-2-yl)-2-(2,6- dioxopiperidin-3-yl)isoindoline-1,3-dione |
| | 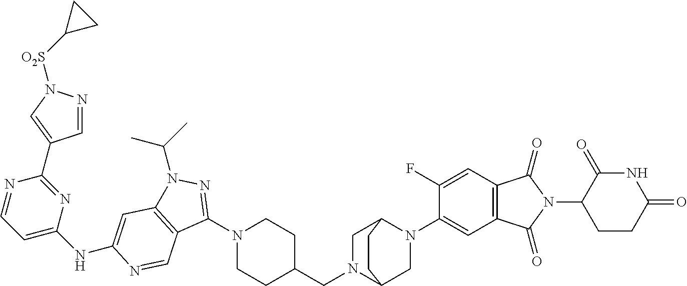 [SMILES: CC(C)n1nc(N2CCC(CN3CC4CCC3CN4c3cc4c(cc3F)C(=O)N(C3CCC(=O)NC3=O)C4=O)CC2)c2cnc(Nc3ccnc(-c4cnn(S(=O)(=O)C5CC5)c4)n3)cc21] | 5-(5-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)-2,5- diazabicyclo[2.2.2]octan-2-yl)-2-(2,6- dioxopiperidin-3-yl)-6-fluoroisoindoline-1,3- dione |
| | 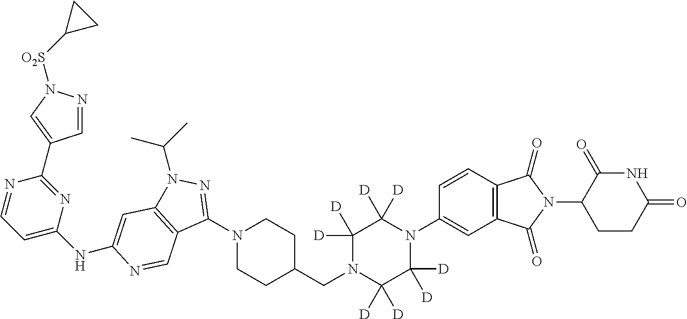 [SMILES: [2H]C1([2H])N(CC2CCN(c3nn(C(C)C)c4cc(Nc5ccnc(-c6cnn(S(=O)(=O)C7CC7)c6)n5)ncc34)CC2)C([2H])([2H])C([2H])([2H])N(c2ccc3c(c2)C(=O)N(C2CCC(=O)NC2=O)C3=O)C1([2H])[2H]] | 5-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)piperazin-1-yl- 2,2,3,3,5,5,6,6-d₈)-2-(2,6-dioxopiperidin-3- yl)isoindoline-1,3-dione |
| | 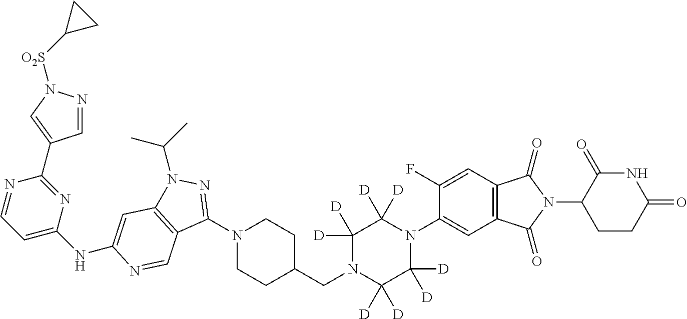 [SMILES: [2H]C1([2H])N(CC2CCN(c3nn(C(C)C)c4cc(Nc5ccnc(-c6cnn(S(=O)(=O)C7CC7)c6)n5)ncc34)CC2)C([2H])([2H])C([2H])([2H])N(c2cc3c(cc2F)C(=O)N(C2CCC(=O)NC2=O)C3=O)C1([2H])[2H]] | 5-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)piperazin-1-yl- 2,2,3,3,5,5,6,6-d₈)-2-(2,6-dioxopiperidin-3-yl)- 6-fluoroisoindoline-1,3-dione |
| | 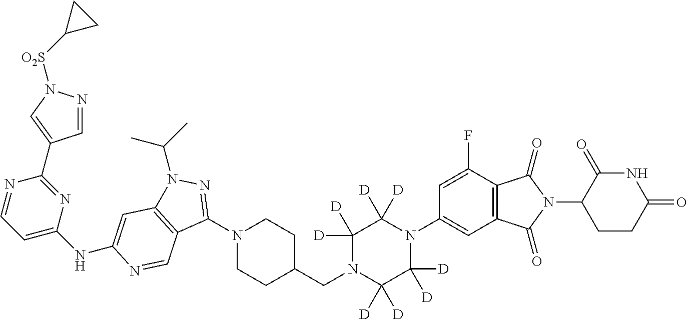 [SMILES: [2H]C1([2H])N(CC2CCN(c3nn(C(C)C)c4cc(Nc5ccnc(-c6cnn(S(=O)(=O)C7CC7)c6)n5)ncc34)CC2)C([2H])([2H])C([2H])([2H])N(c2cc(F)c3c(c2)C(=O)N(C2CCC(=O)NC2=O)C3=O)C1([2H])[2H]] | 6-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)piperazin-1-yl- 2,2,3,3,5,5,6,6-d₈)-2-(2,6-dioxopiperidin-3-yl)- 4-fluoroisoindoline-1,3-dione |
| | 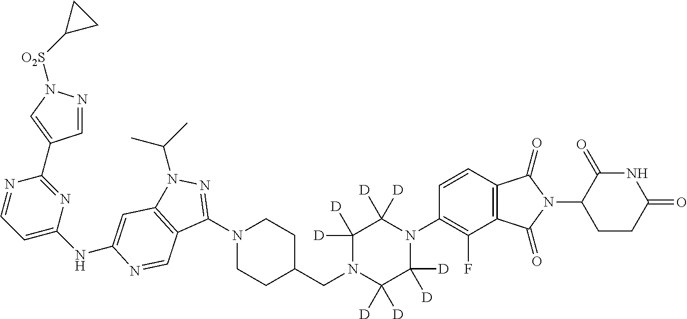 [SMILES: [2H]C1([2H])N(CC2CCN(c3nn(C(C)C)c4cc(Nc5ccnc(-c6cnn(S(=O)(=O)C7CC7)c6)n5)ncc34)CC2)C([2H])([2H])C([2H])([2H])N(c2ccc3c(c2F)C(=O)N(C2CCC(=O)NC2=O)C3=O)C1([2H])[2H]] | 5-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)piperazin-1-yl- 2,2,3,3,5,5,6,6-d₈)-2-(2,6-dioxopiperidin-3-yl)- 4-fluoroisoindoline-1,3-dione |
| | 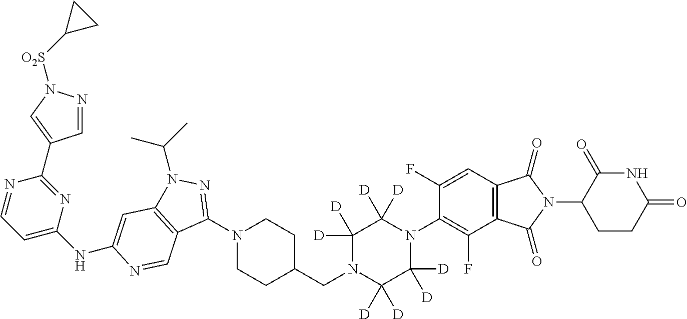 [SMILES: [2H]C1([2H])N(CC2CCN(c3nn(C(C)C)c4cc(Nc5ccnc(-c6cnn(S(=O)(=O)C7CC7)c6)n5)ncc34)CC2)C([2H])([2H])C([2H])([2H])N(c2c(F)cc3c(c2F)C(=O)N(C2CCC(=O)NC2=O)C3=O)C1([2H])[2H]] | 5-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)piperazin-1-yl- 2,2,3,3,5,5,6,6-d₈)-2-(2,6-dioxopiperidin-3-yl)- 4,6-difluoroisoindoline-1,3-dione |
| | 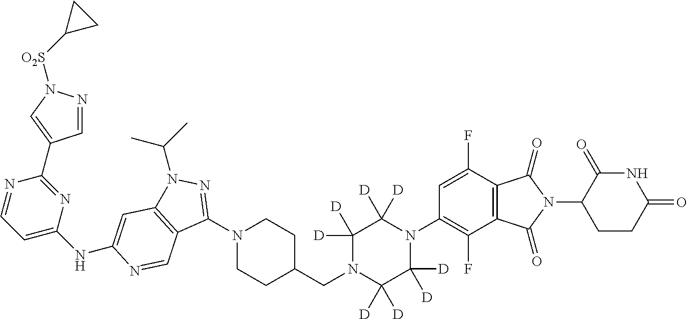 [SMILES: [2H]C1([2H])N(CC2CCN(c3nn(C(C)C)c4cc(Nc5ccnc(-c6cnn(S(=O)(=O)C7CC7)c6)n5)ncc34)CC2)C([2H])([2H])C([2H])([2H])N(c2cc(F)c3c(c2F)C(=O)N(C2CCC(=O)NC2=O)C3=O)C1([2H])[2H]] | 5-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)piperazin-1-yl- 2,2,3,3,5,5,6,6-d₈)-2-(2,6-dioxopiperidin-3-yl)- 4,7-difluoroisoindoline-1,3-dione |
| | 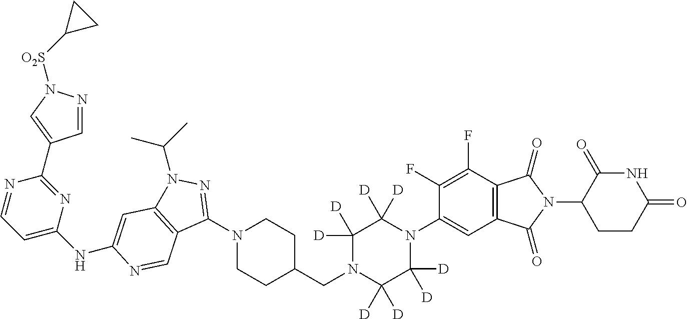 [SMILES: [2H]C1([2H])N(CC2CCN(c3nn(C(C)C)c4cc(Nc5ccnc(-c6cnn(S(=O)(=O)C7CC7)c6)n5)ncc34)CC2)C([2H])([2H])C([2H])([2H])N(c2cc3c(c(F)c2F)C(=O)N(C2CCC(=O)NC2=O)C3=O)C1([2H])[2H]] | 6-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)piperazin-1-yl- 2,2,3,3,5,5,6,6-d₈)-2-(2,6-dioxopiperidin-3-yl)- 4,5-difluoroisoindoline-1,3-dione |
| | 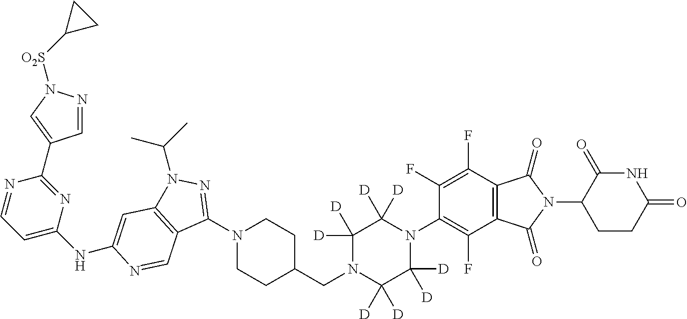 [SMILES: [2H]C1([2H])N(CC2CCN(c3nn(C(C)C)c4cc(Nc5ccnc(-c6cnn(S(=O)(=O)C7CC7)c6)n5)ncc34)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)piperazin-1-yl- 2,2,3,3,5,5,6,6-d₈)-2-(2,6-dioxopiperidin-3-yl)- 4,6,7-trifluoroisoindoline-1,3-dione |
| | 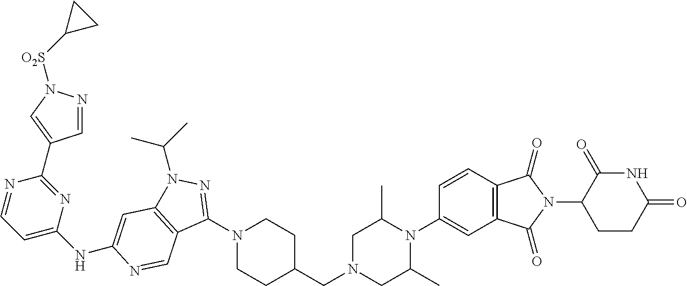 [SMILES: CC1CN(CC2CCN(c3nn(C(C)C)c4cc(Nc5ccnc(-c6cnn(S(=O)(=O)C7CC7)c6)n5)ncc34)CC2)CC(C)N1c1ccc2c(c1)C(=O)N(C1CCC(=O)NC1=O)C2=O] | 5-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)-2,6- dimethylpiperazin-1-yl)-2-(2,6-dioxopiperidin- 3-yl)isoindoline-1,3-dione |
| | 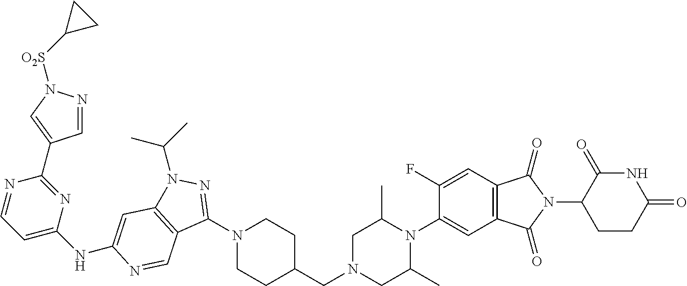 [SMILES: CC1CN(CC2CCN(c3nn(C(C)C)c4cc(Nc5ccnc(-c6cnn(S(=O)(=O)C7CC7)c6)n5)ncc34)CC2)CC(C)N1c1cc2c(cc1F)C(=O)N(C1CCC(=O)NC1=O)C2=O] | 5-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)-2,6- dimethylpiperazin-1-yl)-2-(2,6-dioxopiperidin- 3-yl)-6-fluoroisoindoline-1,3-dione |
| | 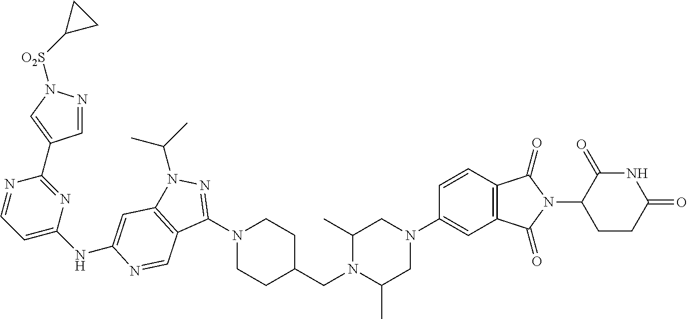 [SMILES: CC1CN(c2ccc3c(c2)C(=O)N(C2CCC(=O)NC2=O)C3=O)CC(C)N1CC1CCN(c2nn(C(C)C)c3cc(Nc4ccnc(-c5cnn(S(=O)(=O)C6CC6)c5)n4)ncc23)CC1] | 5-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)-3,5- dimethylpiperazin-1-yl)-2-(2,6-dioxopiperidin- 3-yl)isoindoline-1,3-dione |
| | 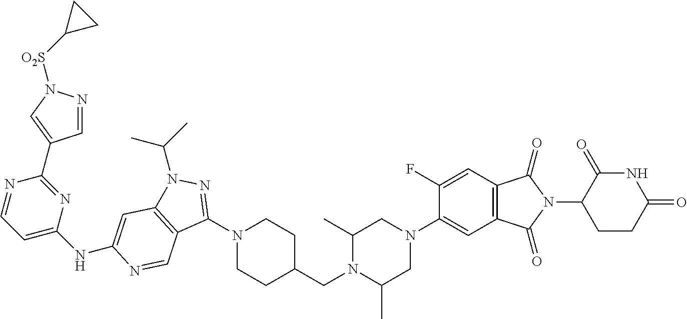 [SMILES: CC1CN(c2cc3c(cc2F)C(=O)N(C2CCC(=O)NC2=O)C3=O)CC(C)N1CC1CCN(c2nn(C(C)C)c3cc(Nc4ccnc(-c5cnn(S(=O)(=O)C6CC6)c5)n4)ncc23)CC1] | 5-(4-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)-3,5- dimethylpiperazin-1-yl)-2-(2,6-dioxopiperidin- 3-yl)-6-fluoroisoindoline-1,3-dione |
| | 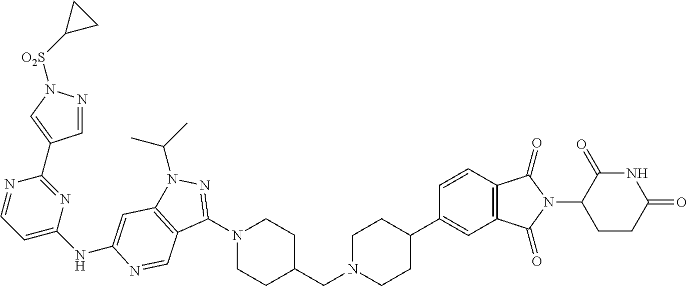 [SMILES: CC(C)n1nc(N2CCC(CN3CCC(c4ccc5c(c4)C(=O)N(C4CCC(=O)NC4=O)C5=O)CC3)CC2)c2cnc(Nc3ccnc(-c4cnn(S(=O)(=O)C5CC5)c4)n3)cc21] | 5-(1-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)piperidin-4-yl)-2-(2,6- dioxopiperidin-3-yl)isoindoline-1,3-dione |
| | 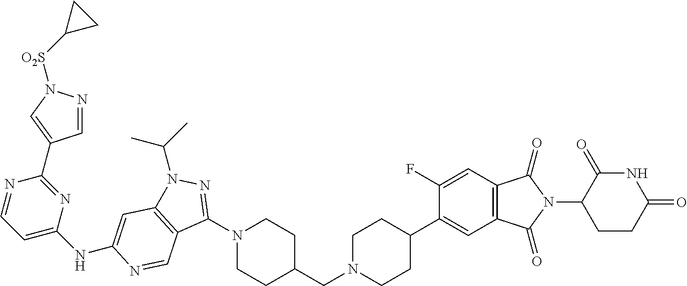 [SMILES: CC(C)n1nc(N2CCC(CN3CCC(c4cc5c(cc4F)C(=O)N(C4CCC(=O)NC4=O)C5=O)CC3)CC2)c2cnc(Nc3ccnc(-c4cnn(S(=O)(=O)C5CC5)c4)n3)cc21] | 5-(1-((1-(6-((2-(1-(cyclopropylsulfonyl)-1H- pyrazol-4-yl)pyrimidin-4-yl)amino)-1- isopropyl-1H-pyrazolo[4,3-c]pyridin-3- yl)piperidin-4-yl)methyl)piperidin-4-yl)-2-(2,6- dioxopiperidin-3-yl)-6-fluoroisoindoline-1,3- dione |
| | 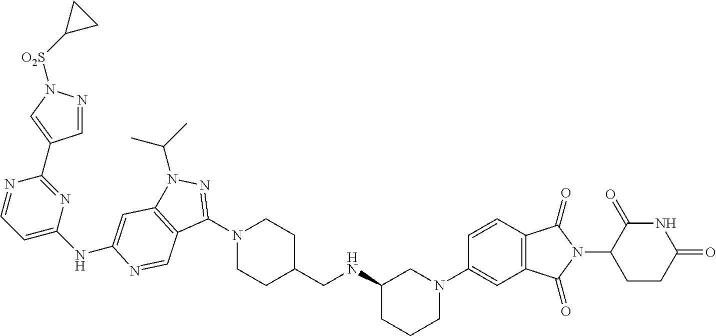 [SMILES: CC(C)n1nc(N2CCC(CN[C@@H]3CCCN(c4ccc5c(c4)C(=O)N(C4CCC(=O)NC4=O)C5=O)C3)CC2)c2cnc(Nc3ccnc(-c4cnn(S(=O)(=O)C5CC5)c4)n3)cc21] | 5-((R)-3-(((1-(6-((2-(1-(cyclopropylsulfonyl)- 1H-pyrazol-4-yl)pyrimidin-4-yl)amino)-1- isopropyl-1H-pyrazolo[4,3-c]pyridin-3- yl)piperidin-4-yl)methyl)amino)piperidin-1-yl)- 2-(2,6-dioxopiperidin-3-yl)isoindoline-1,3- dione |
| | 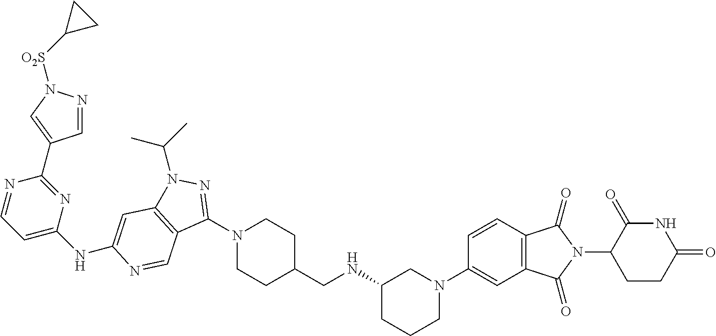 [SMILES: CC(C)n1nc(N2CCC(CN[C@H]3CCCN(c4ccc5c(c4)C(=O)N(C4CCC(=O)NC4=O)C5=O)C3)CC2)c2cnc(Nc3ccnc(-c4cnn(S(=O)(=O)C5CC5)c4)n3)cc21] | 5-((S)-3-(((1-(6-((2-(1-(cyclopropylsulfonyl)- 1H-pyrazol-4-yl)pyrimidin-4-yl)amino)-1- isopropyl-1H-pyrazolo[4,3-c]pyridin-3- yl)piperidin-4-yl)methyl)amino)piperidin-1-yl)- 2-(2,6-dioxopiperidin-3-yl)isoindoline-1,3- dione |
| | 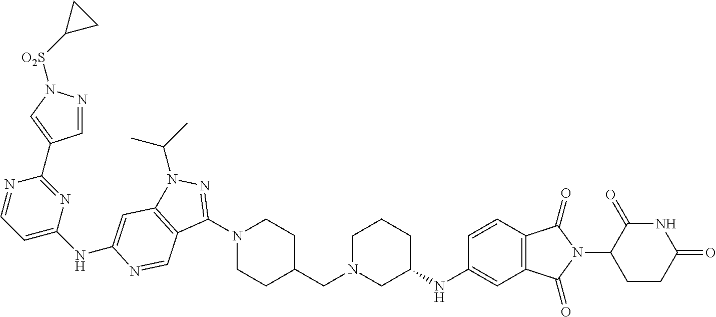 [SMILES: CC(C)n1nc(N2CCC(CN3CCC[C@H](Nc4ccc5c(c4)C(=O)N(C4CCC(=O)NC4=O)C5=O)C3)CC2)c2cnc(Nc3ccnc(-c4cnn(S(=O)(=O)C5CC5)c4)n3)cc21] | 5-(((S)-1-((1-(6-((2-(1-(cyclopropylsulfonyl)- 1H-pyrazol-4-yl)pyrimidin-4-yl)amino)-1- isopropyl-1H-pyrazolo[4,3-c]pyridin-3- yl)piperidin-4-yl)methyl)piperidin-3-yl)amino)- 2-(2,6-dioxopiperidin-3-yl)isoindoline-1,3- dione |
| | 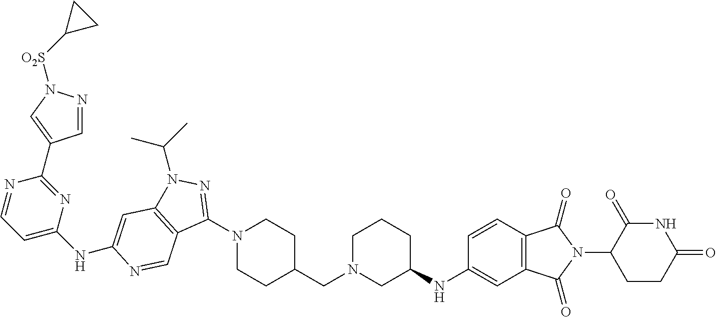 [SMILES: CC(C)n1nc(N2CCC(CN3CCC[C@@H](Nc4ccc5c(c4)C(=O)N(C4CCC(=O)NC4=O)C5=O)C3)CC2)c2cnc(Nc3ccnc(-c4cnn(S(=O)(=O)C5CC5)c4)n3)cc21] | 5-(((R)-1-((1-(6-((2-(1-(cyclopropylsulfonyl)- 1H-pyrazol-4-yl)pyrimidin-4-yl)amino)-1- isopropyl-1H-pyrazolo[4,3-c]pyridin-3- yl)piperidin-4-yl)methyl)piperidin-3-yl)amino)- 2-(2,6-dioxopiperidin-3-yl)isoindoline-1,3- dione |
| | 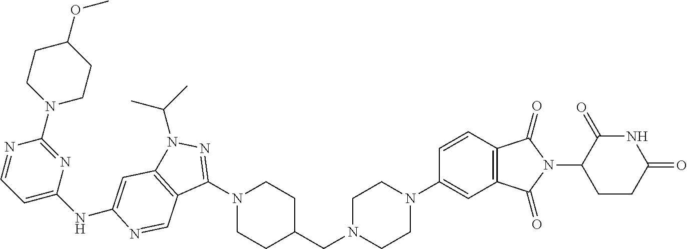 [SMILES: COC1CCN(c2nccc(Nc3cc4c(cn3)c(N3CCC(CN5CCN(c6ccc7c(c6)C(=O)N(C6CCC(=O)NC6=O)C7=O)CC5)CC3)nn4C(C)C)n2)CC1] | 2-(2,6-dioxopiperidin-3-yl)-5-(4-((1-(1- isopropyl-6-((2-(4-methoxypiperidin-1- yl)pyrimidin-4-yl)amino)-1H-pyrazolo[4,3- c]pyridin-3-yl)piperidin-4-yl)methyl)piperazin- 1-yl)isoindoline-1,3-dione |
| | 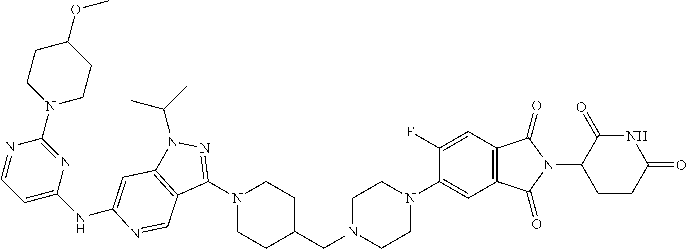 [SMILES: COC1CCN(c2nccc(Nc3cc4c(cn3)c(N3CCC(CN5CCN(c6cc7c(cc6F)C(=O)N(C6CCC(=O)NC6=O)C7=O)CC5)CC3)nn4C(C)C)n2)CC1] | 2-(2,6-dioxopiperidin-3-yl)-5-fluoro-6-(4-((1- (1-isopropyl-6-((2-(4-methoxypiperidin-1- yl)pyrimidin-4-yl)amino)-1H-pyrazolo[4,3- c]pyridin-3-yl)piperidin-4-yl)methyl)piperazin- 1-yl)isoindoline-1,3-dione |
| | 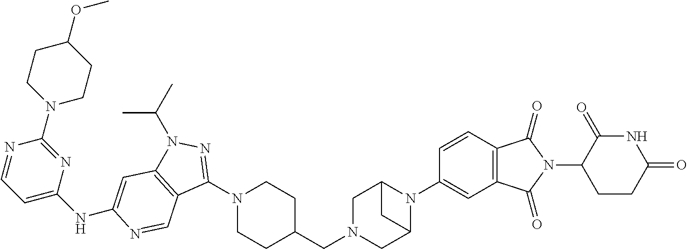 [SMILES: COC1CCN(c2nccc(Nc3cc4c(cn3)c(N3CCC(CN5CC6CC(C5)N6c5ccc6c(c5)C(=O)N(C5CCC(=O)NC5=O)C6=O)CC3)nn4C(C)C)n2)CC1] | 2-(2,6-dioxopiperidin-3-yl)-5-(3-((1-(1- isopropyl-6-((2-(4-methoxypiperidin-1- yl)pyrimidin-4-yl)amino)-1H-pyrazolo[4,3- c]pyridin-3-yl)piperidin-4-yl)methyl)-3,6- diazabicyclo[3.1.1]heptan-6-yl)isoindoline-1,3- dione |
| | 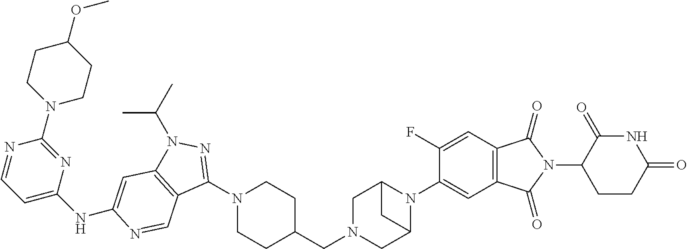 [SMILES: COC1CCN(c2nccc(Nc3cc4c(cn3)c(N3CCC(CN5CC6CC(C5)N6c5cc6c(cc5F)C(=O)N(C5CCC(=O)NC5=O)C6=O)CC3)nn4C(C)C)n2)CC1] | 2-(2,6-dioxopiperidin-3-yl)-5-fluoro-6-(3-((1- (1-isopropyl-6-((2-(4-methoxypiperidin-1- yl)pyrimidin-4-yl)amino)-1H-pyrazolo[4,3- c]pyridin-3-yl)piperidin-4-yl)methyl)-3,6- diazabicyclo[3.1.1]heptan-6-yl)isoindoline-1,3- dione |
| | 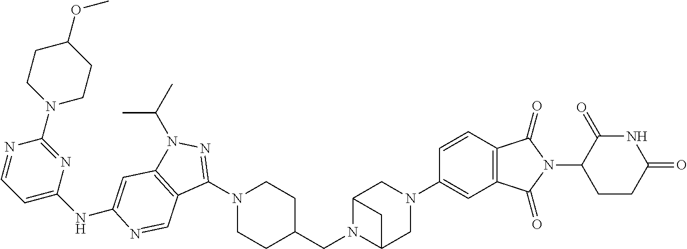 [SMILES: COC1CCN(c2nccc(Nc3cc4c(cn3)c(N3CCC(CN5C6CC5CN(c5ccc7c(c5)C(=O)N(C5CCC(=O)NC5=O)C7=O)C6)CC3)nn4C(C)C)n2)CC1] | 2-(2,6-dioxopiperidin-3-yl)-5-(6-((1-(1- isopropyl-6-((2-(4-methoxypiperidin-1- yl)pyrimidin-4-yl)amino)-1H-pyrazolo[4,3- c]pyridin-3-yl)piperidin-4-yl)methyl)-3,6- diazabicyclo[3.1.1]heptan-3-yl)isoindoline-1,3- dione |
| | 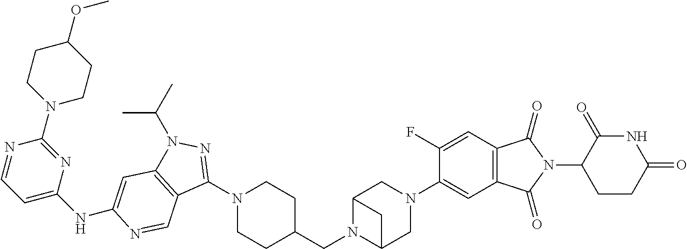 [SMILES: COC1CCN(c2nccc(Nc3cc4c(cn3)c(N3CCC(CN5C6CC5CN(c5cc7c(cc5F)C(=O)N(C5CCC(=O)NC5=O)C7=O)C6)CC3)nn4C(C)C)n2)CC1] | 2-(2,6-dioxopiperidin-3-yl)-5-fluoro-6-(6-((1- (1-isopropyl-6-((2-(4-methoxypiperidin-1- yl)pyrimidin-4-yl)amino)-1H-pyrazolo[4,3- c]pyridin-3-yl)piperidin-4-yl)methyl)-3,6- diazabicyclo[3.1.1]heptan-3-yl)isoindoline-1,3- dione |
| | 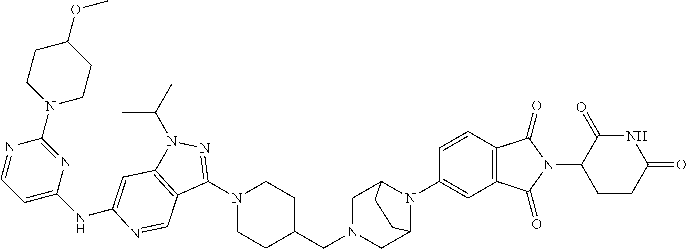 [SMILES: COC1CCN(c2nccc(Nc3cc4c(cn3)c(N3CCC(CN5CC6CCC(C5)N6c5ccc6c(c5)C(=O)N(C5CCC(=O)NC5=O)C6=O)CC3)nn4C(C)C)n2)CC1] | 2-(2,6-dioxopiperidin-3-yl)-5-(3-((1-(1- isopropyl-6-((2-(4-methoxypiperidin-1- yl)pyrimidin-4-yl)amino)-1H-pyrazolo[4,3- c]pyridin-3-yl)piperidin-4-yl)methyl)-3,8- diazabicyclo[3.2.1]octan-8-yl)isoindoline-1,3- dione |
| | 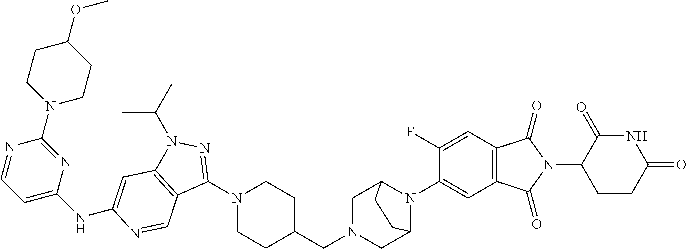 [SMILES: COC1CCN(c2nccc(Nc3cc4c(cn3)c(N3CCC(CN5CC6CCC(C5)N6c5cc6c(cc5F)C(=O)N(C5CCC(=O)NC5=O)C6=O)CC3)nn4C(C)C)n2)CC1] | 2-(2,6-dioxopiperidin-3-yl)-5-fluoro-6-(3-((1- (1-isopropyl-6-((2-(4-methoxypiperidin-1- yl)pyrimidin-4-yl)amino)-1H-pyrazolo[4,3- c]pyridin-3-yl)piperidin-4-yl)methyl)-3,8- diazabicyclo[3.2.1]octan-8-yl)isoindoline-1,3- dione |
| | 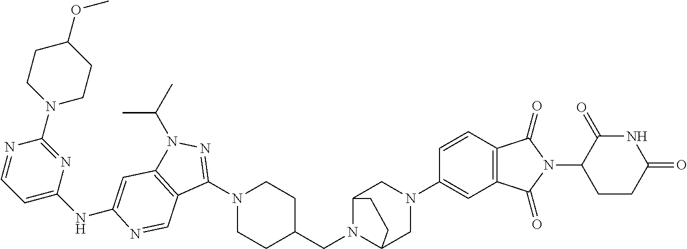 [SMILES: COC1CCN(c2nccc(Nc3cc4c(cn3)c(N3CCC(CN5C6CCC5CN(c5ccc7c(c5)C(=O)N(C5CCC(=O)NC5=O)C7=O)C6)CC3)nn4C(C)C)n2)CC1] | 2-(2,6-dioxopiperidin-3-yl)-5-(8-((1-(1- isopropyl-6-((2-(4-methoxypiperidin-1- yl)pyrimidin-4-yl)amino)-1H-pyrazolo[4,3- c]pyridin-3-yl)piperidin-4-yl)methyl)-3,8- diazabicyclo[3.2.1]octan-3-yl)isoindoline-1,3- dione |
| | 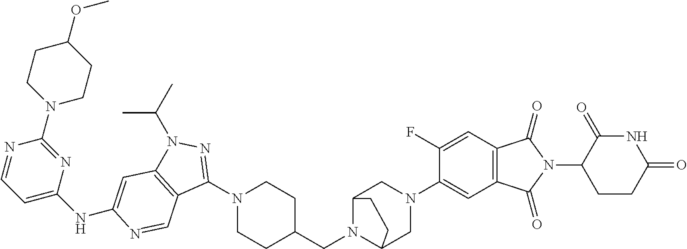 [SMILES: COC1CCN(c2nccc(Nc3cc4c(cn3)c(N3CCC(CN5C6CCC5CN(c5cc7c(cc5F)C(=O)N(C5CCC(=O)NC5=O)C7=O)C6)CC3)nn4C(C)C)n2)CC1] | 2-(2,6-dioxopiperidin-3-yl)-5-fluoro-6-(8-((1- (1-isopropyl-6-((2-(4-methoxypiperidin-1- yl)pyrimidin-4-yl)amino)-1H-pyrazolo[4,3- c]pyridin-3-yl)piperidin-4-yl)methyl)-3,8- diazabicyclo[3.2.1]octan-3-yl)isoindoline-1,3- dione |
| | 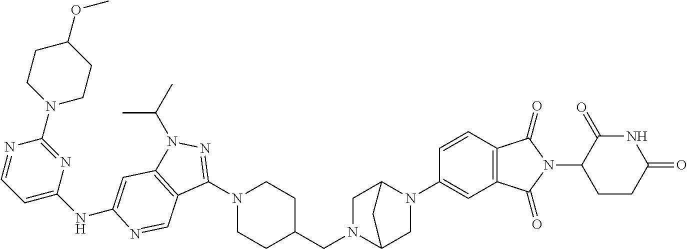 [SMILES: COC1CCN(c2nccc(Nc3cc4c(cn3)c(N3CCC(CN5CC6CC5CN6c5ccc6c(c5)C(=O)N(C5CCC(=O)NC5=O)C6=O)CC3)nn4C(C)C)n2)CC1] | 2-(2,6-dioxopiperidin-3-yl)-5-(5-((1-(1- isopropyl-6-((2-(4-methoxypiperidin-1- yl)pyrimidin-4-yl)amino)-1H-pyrazolo[4,3- c]pyridin-3-yl)piperidin-4-yl)methyl)-2,5- diazabicyclo[2.2.1]heptan-2-yl)isoindoline-1,3- dione |
| | 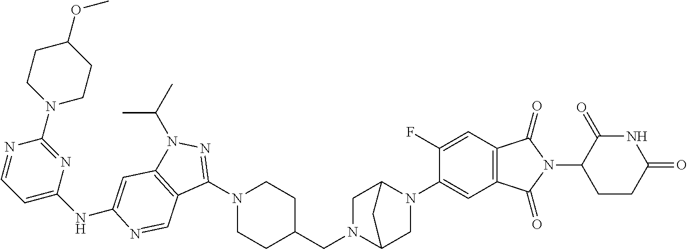 [SMILES: COC1CCN(c2nccc(Nc3cc4c(cn3)c(N3CCC(CN5CC6CC5CN6c5cc6c(cc5F)C(=O)N(C5CCC(=O)NC5=O)C6=O)CC3)nn4C(C)C)n2)CC1] | 2-(2,6-dioxopiperidin-3-yl)-5-fluoro-6-(5-((1- (1-isopropyl-6-((2-(4-methoxypiperidin-1- yl)pyrimidin-4-yl)amino)-1H-pyrazolo[4,3- c]pyridin-3-yl)piperidin-4-yl)methyl)-2,5- diazabicyclo[2.2.1]heptan-2-yl)isoindoline-1,3- dione |
| | 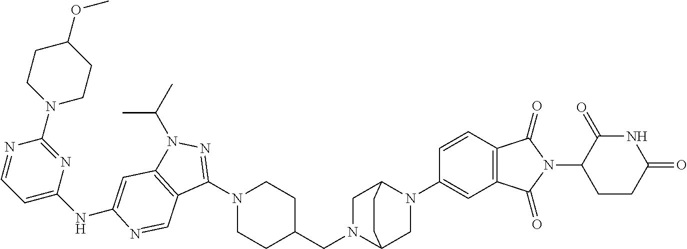 [SMILES: COC1CCN(c2nccc(Nc3cc4c(cn3)c(N3CCC(CN5CC6CCC5CN6c5ccc6c(c5)C(=O)N(C5CCC(=O)NC5=O)C6=O)CC3)nn4C(C)C)n2)CC1] | 2-(2,6-dioxopiperidin-3-yl)-5-(5-((1-(1- isopropyl-6-((2-(4-methoxypiperidin-1- yl)pyrimidin-4-yl)amino)-1H-pyrazolo[4,3- c]pyridin-3-yl)piperidin-4-yl)methyl)-2,5- diazabicyclo[2.2.2]octan-2-yl)isoindoline-1,3- dione |
| | 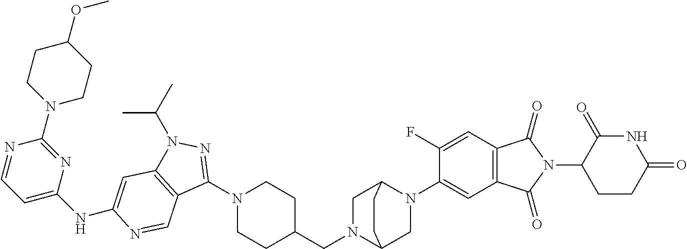 [SMILES: COC1CCN(c2nccc(Nc3cc4c(cn3)c(N3CCC(CN5CC6CCC5CN6c5cc6c(cc5F)C(=O)N(C5CCC(=O)NC5=O)C6=O)CC3)nn4C(C)C)n2)CC1] | 2-(2,6-dioxopiperidin-3-yl)-5-fluoro-6-(5-((1- (1-isopropyl-6-((2-(4-methoxypiperidin-1- yl)pyrimidin-4-yl)amino)-1H-pyrazolo[4,3- c]pyridin-3-yl)piperidin-4-yl)methyl)-2,5- diazabicyclo[2.2.2]octan-2-yl)isoindoline-1,3- dione |
| | 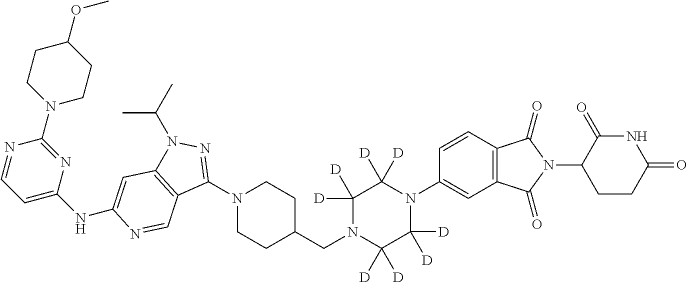 [SMILES: [2H]C1([2H])N(CC2CCN(c3nn(C(C)C)c4cc(Nc5ccnc(N6CCC(OC)CC6)n5)ncc34)CC2)C([2H])([2H])C([2H])([2H])N(c2ccc3c(c2)C(=O)N(C2CCC(=O)NC2=O)C3=O)C1([2H])[2H]] | 2-(2,6-dioxopiperidin-3-yl)-5-(4-((1-(1- isopropyl-6-((2-(4-methoxypiperidin-1- yl)pyrimidin-4-yl)amino)-1H-pyrazolo[4,3- c]pyridin-3-yl)piperidin-4-yl)methyl)piperazin- 1-yl-2,2,3,3,5,5,6,6-d₈)isoindoline-1,3-dione |
| | 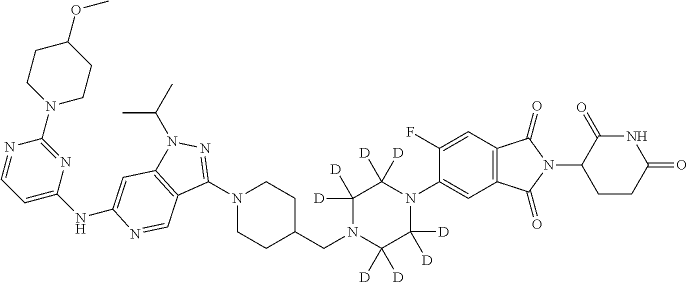 [SMILES: [2H]C1([2H])N(CC2CCN(c3nn(C(C)C)c4cc(Nc5ccnc(N6CCC(OC)CC6)n5)ncc34)CC2)C([2H])([2H])C([2H])([2H])N(c2cc3c(cc2F)C(=O)N(C2CCC(=O)NC2=O)C3=O)C1([2H])[2H]] | 2-(2,6-dioxopiperidin-3-yl)-5-fluoro-6-(4-((1- (1-isopropyl-6-((2-(4-methoxypiperidin-1- yl)pyrimidin-4-yl)amino)-1H-pyrazolo[4,3- c]pyridin-3-yl)piperidin-4-yl)methyl)piperazin- 1-yl-2,2,3,3,5,5,6,6-d₈)isoindoline-1,3-dione |
| | 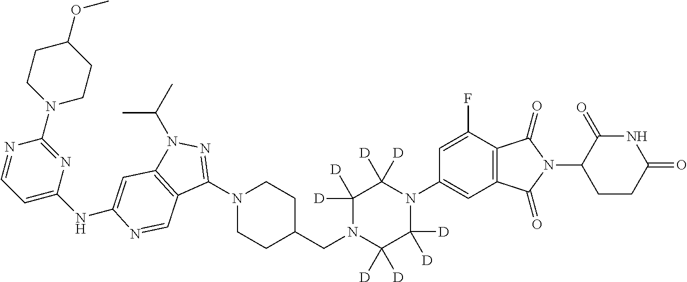 [SMILES: [2H]C1([2H])N(CC2CCN(c3nn(C(C)C)c4cc(Nc5ccnc(N6CCC(OC)CC6)n5)ncc34)CC2)C([2H])([2H])C([2H])([2H])N(c2cc(F)c3c(c2)C(=O)N(C2CCC(=O)NC2=O)C3=O)C1([2H])[2H]] | 2-(2,6-dioxopiperidin-3-yl)-4-fluoro-6-(4-((1- (1-isopropyl-6-((2-(4-methoxypiperidin-1- yl)pyrimidin-4-yl)amino)-1H-pyrazolo[4,3- c]pyridin-3-yl)piperidin-4-yl)methyl)piperazin- 1-yl-2,2,3,3,5,5,6,6-d₈)isoindoline-1,3-dione |
| | 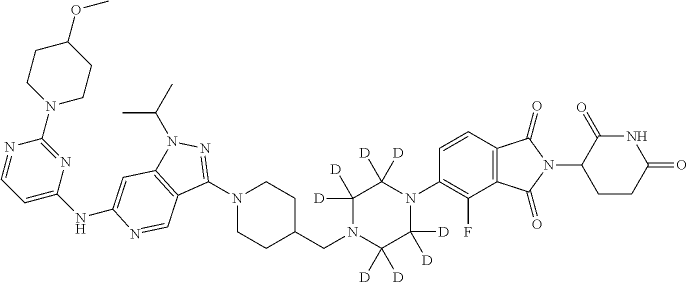 [SMILES: [2H]C1([2H])N(CC2CCN(c3nn(C(C)C)c4cc(Nc5ccnc(N6CCC(OC)CC6)n5)ncc34)CC2)C([2H])([2H])C([2H])([2H])N(c2ccc3c(c2F)C(=O)N(C2CCC(=O)NC2=O)C3=O)C1([2H])[2H]] | 2-(2,6-dioxopiperidin-3-yl)-4-fluoro-5-(4-((1- (1-isopropyl-6-((2-(4-methoxypiperidin-1- yl)pyrimidin-4-yl)amino)-1H-pyrazolo[4,3- c]pyridin-3-yl)piperidin-4-yl)methyl)piperazin- 1-yl-2,2,3,3,5,5,6,6-d₈)isoindoline-1,3-dione |
| | 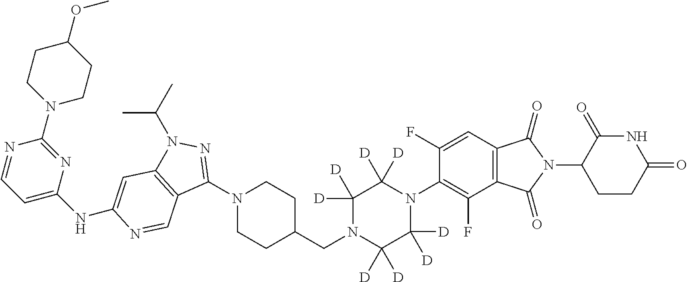 [SMILES: [2H]C1([2H])N(CC2CCN(c3nn(C(C)C)c4cc(Nc5ccnc(N6CCC(OC)CC6)n5)ncc34)CC2)C([2H])([2H])C([2H])([2H])N(c2c(F)cc3c(c2F)C(=O)N(C2CCC(=O)NC2=O)C3=O)C1([2H])[2H]] | 2-(2,6-dioxopiperidin-3-yl)-4,6-difluoro-5-(4- ((1-(1-isopropyl-6-((2-(4-methoxypiperidin-1- yl)pyrimidin-4-yl)amino)-1H-pyrazolo[4,3- c]pyridin-3-yl)piperidin-4-yl)methyl)piperazin- 1-yl-2,2,3,3,5,5,6,6-d₈)isoindoline-1,3-dione |
| | 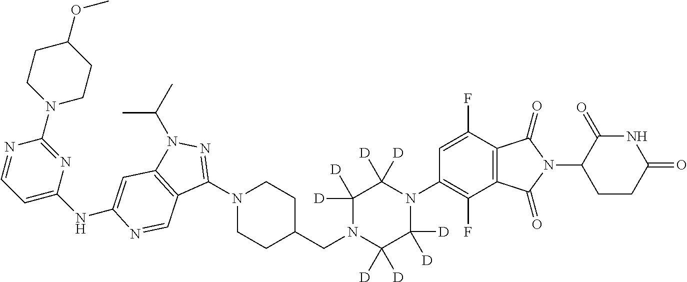 [SMILES: [2H]C1([2H])N(CC2CCN(c3nn(C(C)C)c4cc(Nc5ccnc(N6CCC(OC)CC6)n5)ncc34)CC2)C([2H])([2H])C([2H])([2H])N(c2cc(F)c3c(c2F)C(=O)N(C2CCC(=O)NC2=O)C3=O)C1([2H])[2H]] | 2-(2,6-dioxopiperidin-3-yl)-4,7-difluoro-5-(4- ((1-(1-isopropyl-6-((2-(4-methoxypiperidin-1- yl)pyrimidin-4-yl)amino)-1H-pyrazolo[4,3- c]pyridin-3-yl)piperidin-4-yl)methyl)piperazin- 1-yl-2,2,3,3,5,5,6,6-d₈)isoindoline-1,3-dione |
| | 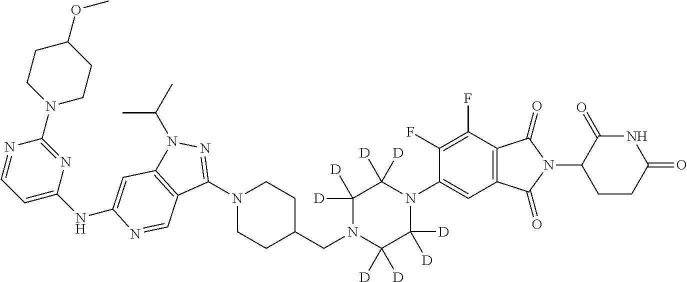 [SMILES: [2H]C1([2H])N(CC2CCN(c3nn(C(C)C)c4cc(Nc5ccnc(N6CCC(OC)CC6)n5)ncc34)CC2)C([2H])([2H])C([2H])([2H])N(c2cc3c(c(F)c2F)C(=O)N(C2CCC(=O)NC2=O)C3=O)C1([2H])[2H]] | 2-(2,6-dioxopiperidin-3-yl)-4,5-difluoro-6-(4- ((1-(1 -isopropyl-6-((2-(4-methoxypiperidin-1- yl)pyrimidin-4-yl)amino)-1H-pyrazolo[4,3- c]pyridin-3-yl)piperidin-4-yl)methyl)piperazin- 1-yl-2,2,3,3,5,5,6,6-d₈)isoindoline-1,3-dione |
| | 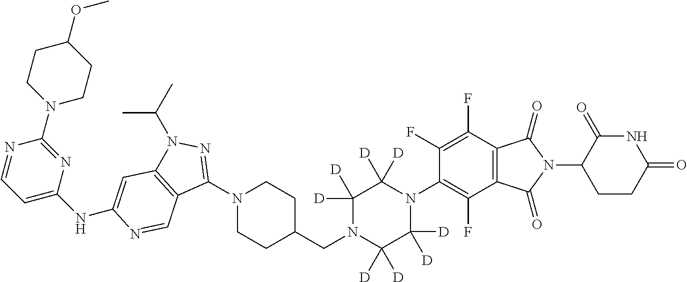 [SMILES: [2H]C1([2H])N(CC2CCN(c3nn(C(C)C)c4cc(Nc5ccnc(N6CCC(OC)CC6)n5)ncc34)CC2)C([2H])([2H])C([2H])([2H])N(c2c(F)c(F)c3c(c2F)C(=O)N(C2CCC(=O)NC2=O)C3=O)C1([2H])[2H]] | 2-(2,6-dioxopiperidin-3-yl)-4,5,7-trifluoro-6- (4-((1-(1-isopropyl-6-((2-(4-methoxypiperidin- 1-yl)pyrimidin-4-yl)amino)-1H-pyrazolo[4,3- c]pyridin-3-yl)piperidin-4-yl)methyl)piperazin- 1-yl-2,2,3,3,5,5,6,6-d₈)isoindoline-1,3-dione |
| | 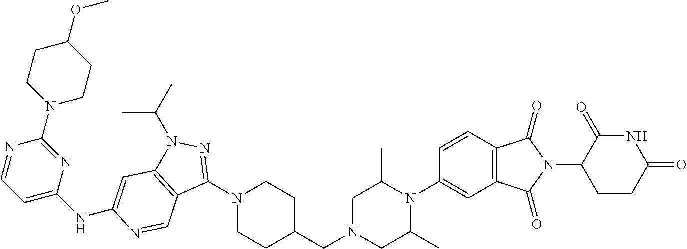 [SMILES: COC1CCN(c2nccc(Nc3cc4c(cn3)c(N3CCC(CN5CC(C)N(c6ccc7c(c6)C(=O)N(C6CCC(=O)NC6=O)C7=O)C(C)C5)CC3)nn4C(C)C)n2)CC1] | 2-(2,6-dioxopiperidin-3-yl)-5-(4-((1-(1- isopropyl-6-((2-(4-methoxypiperidin-1- yl)pyrimidin-4-yl)amino)-1H-pyrazolo[4,3- c]pyridin-3-yl)piperidin-4-yl)methyl)-2,6- dimethylpiperazin-1-yl)isoindoline-1,3-dione |
| | 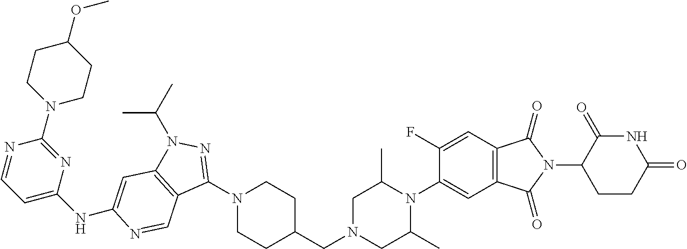 [SMILES: COC1CCN(c2nccc(Nc3cc4c(cn3)c(N3CCC(CN5CC(C)N(c6cc7c(cc6F)C(=O)N(C6CCC(=O)NC6=O)C7=O)C(C)C5)CC3)nn4C(C)C)n2)CC1] | 2-(2,6-dioxopiperidin-3-yl)-5-fluoro-6-(4-((1- (1-isopropyl-6-((2-(4-methoxypiperidin-1- yl)pyrimidin-4-yl)amino)-1H-pyrazolo[4,3- c]pyridin-3-yl)piperidin-4-yl)methyl)-2,6- dimethylpiperazin-1-yl)isoindoline-1,3-dione |
| | 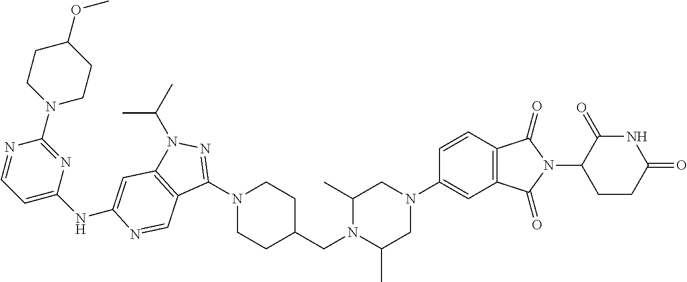 [SMILES: COC1CCN(c2nccc(Nc3cc4c(cn3)c(N3CCC(CN5C(C)CN(c6ccc7c(c6)C(=O)N(C6CCC(=O)NC6=O)C7=O)CC5C)CC3)nn4C(C)C)n2)CC1] | 2-(2,6-dioxopiperidin-3-yl)-5-(4-((1-(1- isopropyl-6-((2-(4-methoxypiperidin-1- yl)pyrimidin-4-yl)amino)-1H-pyrazolo[4,3- c]pyridin-3-yl)piperidin-4-yl)methyl)-3,5- dimethylpiperazin-1-yl)isoindoline-1,3-dione |
| | 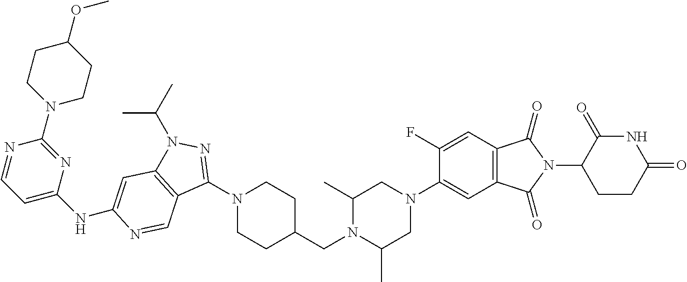 [SMILES: COC1CCN(c2nccc(Nc3cc4c(cn3)c(N3CCC(CN5C(C)CN(c6cc7c(cc6F)C(=O)N(C6CCC(=O)NC6=O)C7=O)CC5C)CC3)nn4C(C)C)n2)CC1] | 2-(2,6-dioxopiperidin-3-yl)-5-fluoro-6-(4-((1- (1-isopropyl-6-((2-(4-methoxypiperidin-1- yl)pyrimidin-4-yl)amino)-1H-pyrazolo[4,3- c]pyridin-3-yl)piperidin-4-yl)methyl)-3,5- dimethylpiperazin-1-yl)isoindoline-1,3-dione |
| | 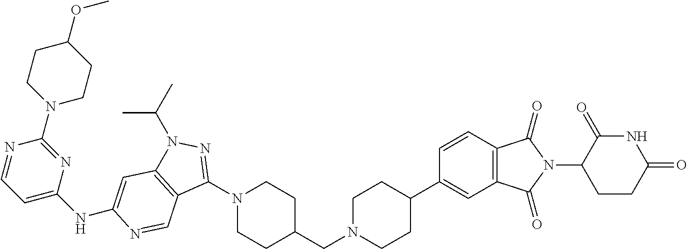 [SMILES: COC1CCN(c2nccc(Nc3cc4c(cn3)c(N3CCC(CN5CCC(c6ccc7c(c6)C(=O)N(C6CCC(=O)NC6=O)C7=O)CC5)CC3)nn4C(C)C)n2)CC1] | 2-(2,6-dioxopiperidin-3-yl)-5-(1-((1-(1- isopropyl-6-((2-(4-methoxypiperidin-1- yl)pyrimidin-4-yl)amino)-1H-pyrazolo[4,3- c]pyridin-3-yl)piperidin-4-yl)methyl)piperidin- 4-yl)isoindoline-1,3-dione |
| | 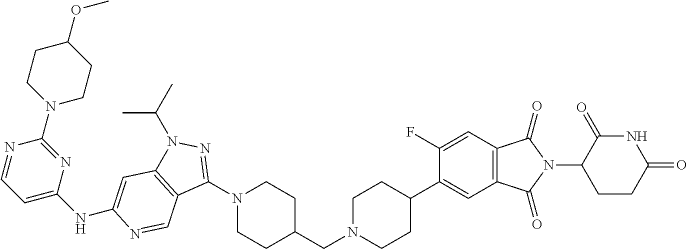 [SMILES: COC1CCN(c2nccc(Nc3cc4c(cn3)c(N3CCC(CN5CCC(c6cc7c(cc6F)C(=O)N(C6CCC(=O)NC6=O)C7=O)CC5)CC3)nn4C(C)C)n2)CC1] | 2-(2,6-dioxopiperidin-3-yl)-5-fluoro-6-(1-((1- (1-isopropyl-6-((2-(4-methoxypiperidin-1- yl)pyrimidin-4-yl)amino)-1H-pyrazolo[4,3- c]pyridin-3-yl)piperidin-4-yl)methyl)piperidin- 4-yl)isoindoline-1,3-dione |
| | 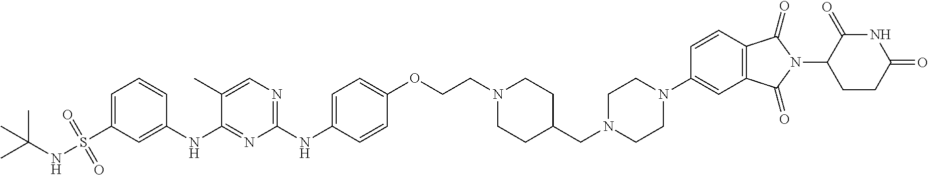 [SMILES: Cc1cnc(Nc2ccc(OCCN3CCC(CN4CCN(c5ccc6c(c5)C(=O)N(C5CCC(=O)NC5=O)C6=O)CC4)CC3)cc2)nc1Nc1cccc(S(=O)(=O)NC(C)(C)C)c1] | N-(tert-butyl)-3-((2-((4-(2-(4-((4-(2-(2,6- dioxopiperidin-3-yl)-1,3-dioxoisoindolin-5- yl)piperazin-1-yl)methyl)piperidin-1- yl)ethoxy)phenyl)amino)-5-methylpyrimidin-4- yl)amino)benzenesulfonamide |
| | 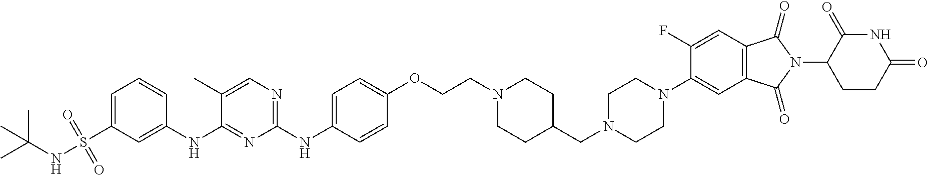 [SMILES: Cc1cnc(Nc2ccc(OCCN3CCC(CN4CCN(c5cc6c(cc5F)C(=O)N(C5CCC(=O)NC5=O)C6=O)CC4)CC3)cc2)nc1Nc1cccc(S(=O)(=O)NC(C)(C)C)c1] | N-(tert-butyl)-3-((2-((4-(2-(4-((4-(2-(2,6- dioxopiperidin-3-yl)-6-fluoro-1,3- dioxoisoindolin-5-yl)piperazin-1- yl)methyl)piperidin-1- yl)ethoxy)phenyl)amino)-5-methylpyrimidin-4- yl)amino)benzenesulfonamide |
| | 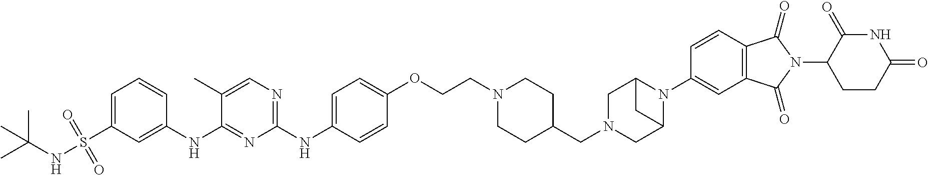 [SMILES: Cc1cnc(Nc2ccc(OCCN3CCC(CN4CC5CC(C4)N5c4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(2-(4-((6-(2-(2,6- dioxopiperidin-3-yl)-1,3-dioxoisoindolin-5-yl)- 3,6-diazabicyclo[3.1.1]heptan-3- yl)methyl)piperidin-1- yl)ethoxy)phenyl)amino)-5-methylpyrimidin-4- yl)amino)benzenesulfonamide |
| | 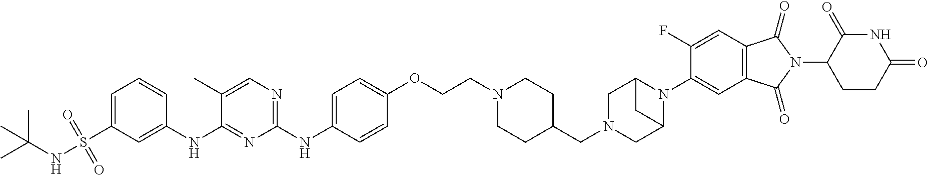 [SMILES: Cc1cnc(Nc2ccc(OCCN3CCC(CN4CC5CC(C4)N5c4cc5c(cc4F)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(2-(4-((6-(2-(2,6- dioxopiperidin-3-yl)-6-fluoro-1,3- dioxoisoindolin-5-yl)-3,6- diazabicyclo[3.1.1]heptan-3- yl)methyl)piperidin-1- yl)ethoxy)phenyl)amino)-5-methylpyrimidin-4- yl)amino)benzenesulfonamide |
| | 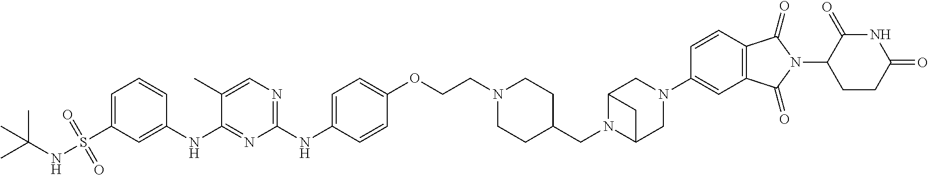 [SMILES: Cc1cnc(Nc2ccc(OCCN3CCC(CN4C5CC4CN(c4ccc6c(c4)C(=O)N(C4CCC(=O)NC4=O)C6=O)C5)CC3)cc2)nc1Nc1cccc(S(=O)(=O)NC(C)(C)C)c1] | N-(tert-butyl)-3-((2-((4-(2-(4-((3-(2-(2,6- dioxopiperidin-3-yl)-1,3-dioxoisoindolin-5-yl)- 3,6-diazabicyclo[3.1.1]heptan-6- yl)methyl)piperidin-1- yl)ethoxy)phenyl)amino)-5-methylpyrimidin-4- yl)amino)benzenesulfonamide |
| | 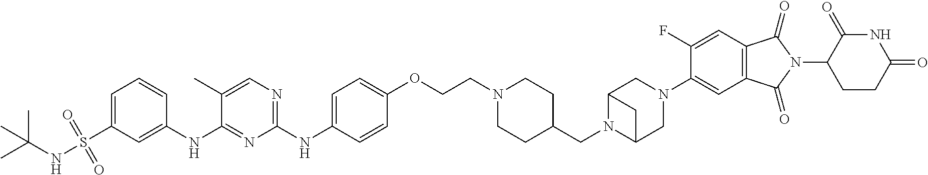 [SMILES: Cc1cnc(Nc2ccc(OCCN3CCC(CN4C5CC4CN(c4cc6c(cc4F)C(=O)N(C4CCC(=O)NC4=O)C6=O)C5)CC3)cc2)nc1Nc1cccc(S(=O)(=O)NC(C)(C)C)c1] | N-(tert-butyl)-3-((2-((4-(2-(4-((3-(2-(2,6- dioxopiperidin-3-yl)-6-fluoro-1,3- dioxoisoindolin-5-yl)-3,6- diazabicyclo[3.1.1]heptan-6- yl)methyl)piperidin-1- yl)ethoxy)phenyl)amino)-5-methylpyrimidin-4- yl)amino)benzenesulfonamide |
| | 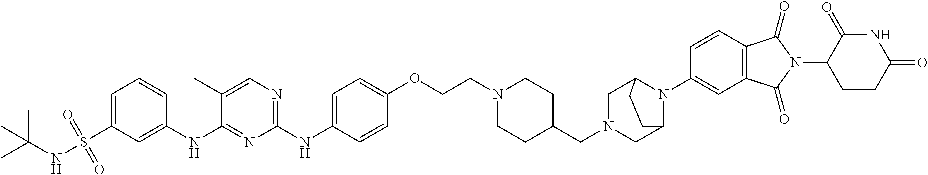 [SMILES: Cc1cnc(Nc2ccc(OCCN3CCC(CN4CC5CCC(C4)N5c4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(2-(4-((8-(2-(2,6- dioxopiperidin-3-yl)-1,3-dioxoisoindolin-5-yl)- 3,8-diazabicyclo[3.2.1]octan-3- yl)methyl)piperidin-1- yl)ethoxy)phenyl)amino)-5-methylpyrimidin-4- yl)amino)benzenesulfonamide |
| | 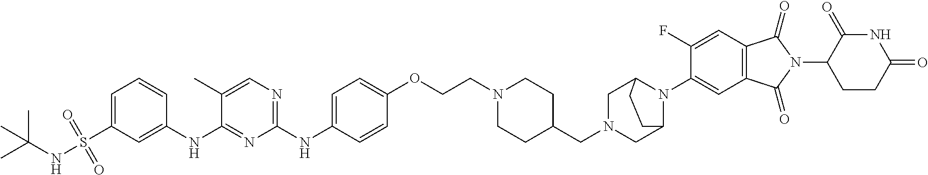 [SMILES: Cc1cnc(Nc2ccc(OCCN3CCC(CN4CC5CCC(C4)N5c4cc5c(cc4F)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(2-(4-((8-(2-(2,6- dioxopiperidin-3-yl)-6-fluoro-1,3- dioxoisoindolin-5-yl)-3,8- diazabicyclo[3.2.1]octan-3-yl)methyl)piperidin- 1-yl)ethoxy)phenyl)amino)-5-methylpyrimidin- 4-yl)amino)benzenesulfonamide |
| | 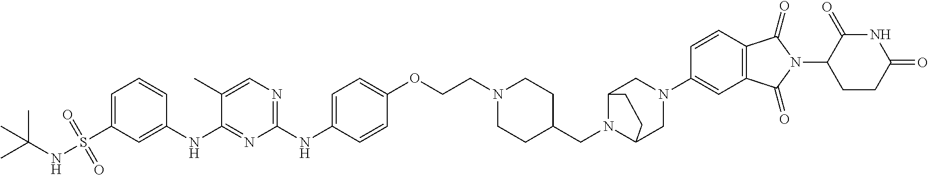 [SMILES: Cc1cnc(Nc2ccc(OCCN3CCC(CN4C5CCC4CN(c4ccc6c(c4)C(=O)N(C4CCC(=O)NC4=O)C6=O)C5)CC3)cc2)nc1Nc1cccc(S(=O)(=O)NC(C)(C)C)c1] | N-(tert-butyl)-3-((2-((4-(2-(4-((3-(2-(2,6- dioxopiperidin-3-yl)-1,3-dioxoisoindolin-5-yl)- 3,8-diazabicyclo[3.2.1]octan-8- yl)methyl)piperidin-1- yl)ethoxy)phenyl)amino)-5-methylpyrimidin-4- yl)amino)benzenesulfonamide |
| | 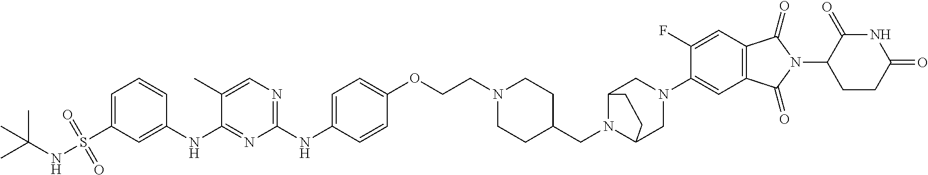 [SMILES: Cc1cnc(Nc2ccc(OCCN3CCC(CN4C5CCC4CN(c4cc6c(cc4F)C(=O)N(C4CCC(=O)NC4=O)C6=O)C5)CC3)cc2)nc1Nc1cccc(S(=O)(=O)NC(C)(C)C)c1] | N-(tert-butyl)-3-((2-((4-(2-(4-((3-(2-(2,6- dioxopiperidin-3-yl)-6-fluoro-1,3- dioxoisoindolin-5-yl)-3,8- diazabicyclo[3.2.1]octan-8-yl)methyl)piperidin- 1-yl)ethoxy)phenyl)amino)-5-methylpyrimidin- 4-yl)amino)benzenesulfonamide |
| | 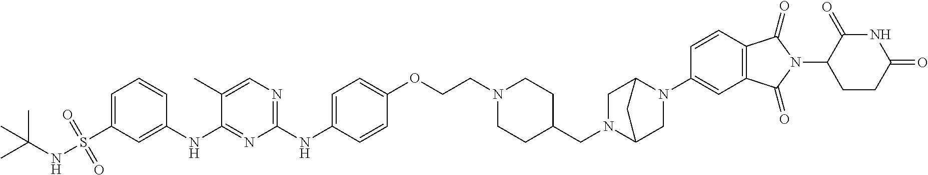 [SMILES: Cc1cnc(Nc2ccc(OCCN3CCC(CN4CC5CC4CN5c4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(2-(4-((5-(2-(2,6- dioxopiperidin-3-yl)-1,3-dioxoisoindolin-5-yl)- 2,5-diazabicyclo[2.2.1]heptan-2- yl)methyl)piperidin-1- yl)ethoxy)phenyl)amino)-5-methylpyrimidin-4- yl)amino)benzenesulfonamide |
| | 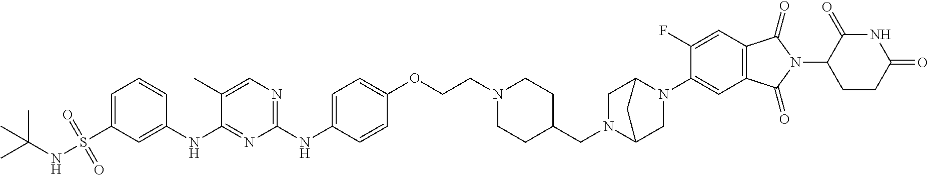 [SMILES: Cc1cnc(Nc2ccc(OCCN3CCC(CN4CC5CC4CN5c4cc5c(cc4F)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(2-(4-((5-(2-(2,6- dioxopiperidin-3-yl)-6-fluoro-1,3- dioxoisoindolin-5-yl)-2,5- diazabicyclo[2.2.1]heptan-2- yl)methyl)piperidin-1- yl)ethoxy)phenyl)amino)-5-methylpyrimidin-4- yl)amino)benzenesulfonamide |
| | 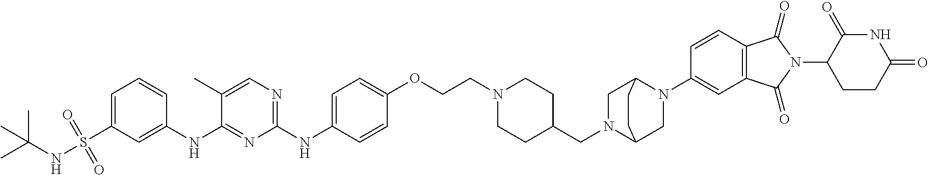 [SMILES: Cc1cnc(Nc2ccc(OCCN3CCC(CN4CC5CCC4CN5c4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(2-(4-((5-(2-(2,6- dioxopiperidin-3-yl)-1,3-dioxoisoindolin-5-yl)- 2,5-diazabicyclo[2.2.2]octan-2- yl)methyl)piperidin-1- yl)ethoxy)phenyl)amino)-5-methylpyrimidin-4- yl)amino)benzenesulfonamide |
| | 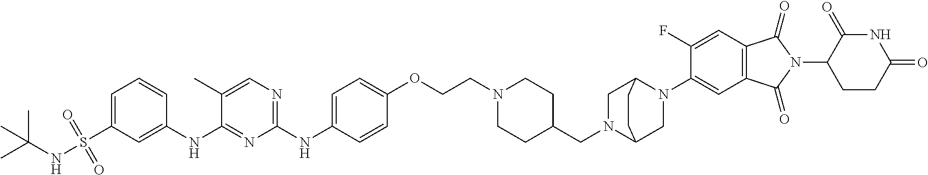 [SMILES: Cc1cnc(Nc2ccc(OCCN3CCC(CN4CC5CCC4CN5c4cc5c(cc4F)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(2-(4-((5-(2-(2,6- dioxopiperidin-3-yl)-6-fluoro-1,3- dioxoisoindolin-5-yl)-2,5- diazabicyclo[2.2.2]octan-2-yl)methyl)piperidin- 1-yl)ethoxy)phenyl)amino)-5-methylpyrimidin- 4-yl)amino)benzenesulfonamide |
| | 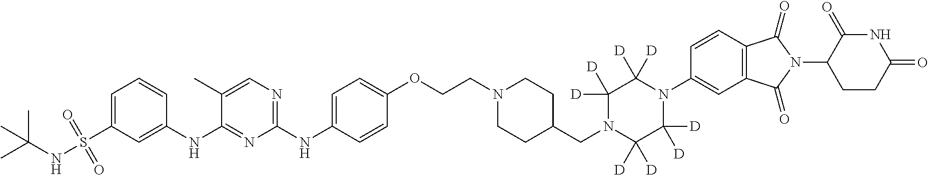 [SMILES: [2H]C1([2H])N(CC2CCN(CCOc3ccc(Nc4ncc(C)c(Nc5cccc(S(=O)(=O)NC(C)(C)C)c5)n4)cc3)CC2)C([2H])([2H])C([2H])([2H])N(c2ccc3c(c2)C(=O)N(C2CCC(=O)NC2=O)C3=O)C1([2H])[2H]] | N-(tert-butyl)-3-((2-((4-(2-(4-((4-(2-(2,6- dioxopiperidin-3-yl)-1,3-dioxoisoindolin-5- yl)piperazin-1-yl-2,2,3,3,5,5,6,6- d₈)methyl)piperidin-1- yl)ethoxy)phenyl)amino)-5-methylpyrimidin-4- yl)amino)benzenesulfonamide |
| | 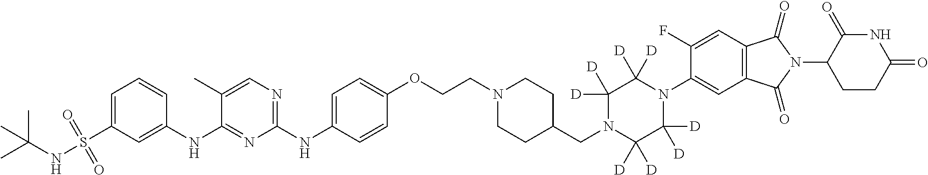 [SMILES: [2H]C1([2H])N(CC2CCN(CCOc3ccc(Nc4ncc(C)c(Nc5cccc(S(=O)(=O)NC(C)(C)C)c5)n4)cc3)CC2)C([2H])([2H])C([2H])([2H])N(c2cc3c(cc2F)C(=O)N(C2CCC(=O)NC2=O)C3=O)C1([2H])[2H]] | N-(tert-butyl)-3-((2-((4-(2-(4-((4-(2-(2,6- dioxopiperidin-3-yl)-6-fluoro-1,3- dioxoisoindolin-5-yl)piperazin-1-yl- 2,2,3,3,5,5,6,6-d₈)methyl)piperidin-1- yl)ethoxy)phenyl)amino)-5-methylpyrimidin-4- yl)amino)benzenesulfonamide |
| | 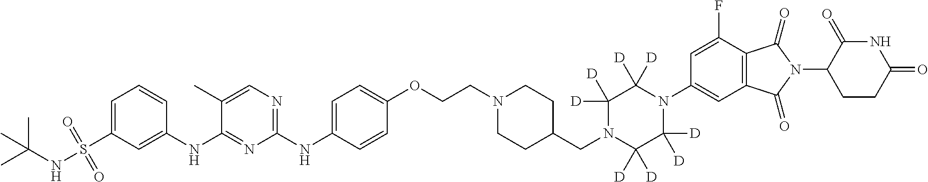 [SMILES: [2H]C1([2H])N(CC2CCN(CCOc3ccc(Nc4ncc(C)c(Nc5cccc(S(=O)(=O)NC(C)(C)C)c5)n4)cc3)CC2)C([2H])([2H])C([2H])([2H])N(c2cc(F)c3c(c2)C(=O)N(C2CCC(=O)NC2=O)C3=O)C1([2H])[2H]] | N-(tert-butyl)-3-((2-((4-(2-(4-((4-(2-(2,6- dioxopiperidin-3-yl)-7-fluoro-1,3- dioxoisoindolin-5-yl)piperazin-1-yl- 2,2,3,3,5,5,6,6-d₈)methyl)piperidin-1- yl)ethoxy)phenyl)amino)-5-methylpyrimidin-4- yl)amino)benzenesulfonamide |
| | 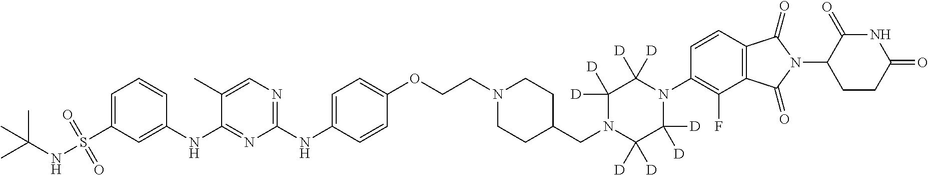 [SMILES: [2H]C1([2H])N(CC2CCN(CCOc3ccc(Nc4ncc(C)c(Nc5cccc(S(=O)(=O)NC(C)(C)C)c5)n4)cc3)CC2)C([2H])([2H])C([2H])([2H])N(c2ccc3c(c2F)C(=O)N(C2CCC(=O)NC2=O)C3=O)C1([2H])[2H]] | N-(tert-butyl)-3-((2-((4-(2-(4-((4-(2-(2,6- dioxopiperidin-3-yl)-4-fluoro-1,3- dioxoisoindolin-5-yl)piperazin-1-yl- 2,2,3,3,5,5,6,6-d₈)methyl)piperidin-1- yl)ethoxy)phenyl)amino)-5-methylpyrimidin-4- yl)amino)benzenesulfonamide |
| | 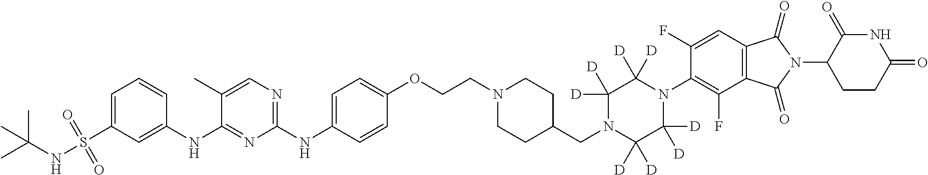 [SMILES: [2H]C1([2H])N(CC2CCN(CCOc3ccc(Nc4ncc(C)c(Nc5cccc(S(=O)(=O)NC(C)(C)C)c5)n4)cc3)CC2)C([2H])([2H])C([2H])([2H])N(c2c(F)cc3c(c2F)C(=O)N(C2CCC(=O)NC2=O)C3=O)C1([2H])[2H]] | N-(tert-butyl)-3-((2-((4-(2-(4-((4-(2-(2,6- dioxopiperidin-3-yl)-4,6-difluoro-1,3- dioxoisoindolin-5-yl)piperazin-1-yl- 2,2,3,3,5,5,6,6-d₈)methyl)piperidin-1- yl)ethoxy)phenyl)amino)-5-methylpyrimidin-4- yl)amino)benzenesulfonamide |
| | 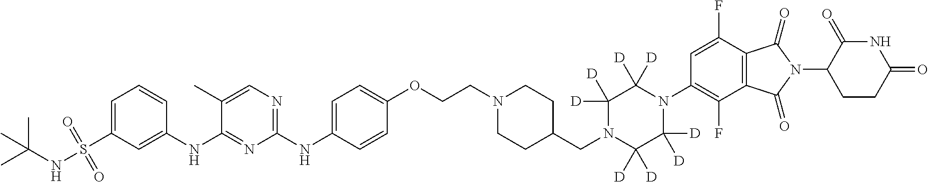 [SMILES: [2H]C1([2H])N(CC2CCN(CCOc3ccc(Nc4ncc(C)c(Nc5cccc(S(=O)(=O)NC(C)(C)C)c5)n4)cc3)CC2)C([2H])([2H])C([2H])([2H])N(c2cc(F)c3c(c2F)C(=O)N(C2CCC(=O)NC2=O)C3=O)C1([2H])[2H]] | N-(tert-butyl)-3-((2-((4-(2-(4-((4-(2-(2,6- dioxopiperidin-3-yl)-4,7-difluoro-1,3- dioxoisoindolin-5-yl)piperazin-1-yl- 2,2,3,3,5,5,6,6-d₈)methyl)piperidin-1- yl)ethoxy)phenyl)amino)-5-methylpyrimidin-4- yl)amino)benzenesulfonamide |
| | 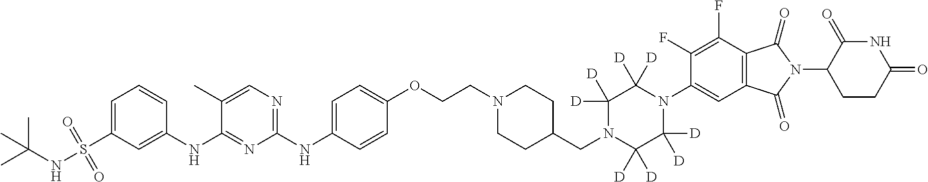 [SMILES: [2H]C1([2H])N(CC2CCN(CCOc3ccc(Nc4ncc(C)c(Nc5cccc(S(=O)(=O)NC(C)(C)C)c5)n4)cc3)CC2)C([2H])([2H])C([2H])([2H])N(c2cc3c(c(F)c2F)C(=O)N(C2CCC(=O)NC2=O)C3=O)C1([2H])[2H]] | N-(tert-butyl)-3-((2-((4-(2-(4-((4-(2-(2,6- dioxopiperidin-3-yl)-6,7-difluoro-1,3- dioxoisoindolin-5-yl)piperazin-1-yl- 2,2,3,3,5,5,6,6-d₈)methyl)piperidin-1- yl)ethoxy)phenyl)amino)-5-methylpyrimidin-4- yl)amino)benzenesulfonamide |
| | 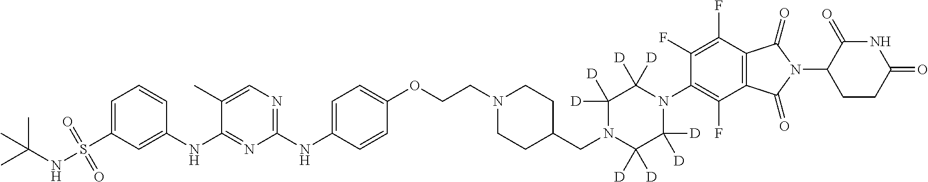 [SMILES: [2H]C1([2H])N(CC2CCN(CCOc3ccc(Nc4ncc(C)c(Nc5cccc(S(=O)(=O)NC(C)(C)C)c5)n4)cc3)CC2)C([2H])([2H])C([2H])([2H])N(c2c(F)c(F)c3c(c2F)C(=O)N(C2CCC(=O)NC2=O)C3=O)C1([2H])[2H]] | N-(tert-butyl)-3-((2-((4-(2-(4-((4-(2-(2,6- dioxopiperidin-3-yl)-4,6,7-trifluoro-1,3- dioxoisoindolin-5-yl)piperazin-1-yl- 2,2,3,3,5,5,6,6-d₈)methyl)piperidin-1- yl)ethoxy)phenyl)amino)-5-methylpyrimidin-4- yl)amino)benzenesulfonamide |
| | 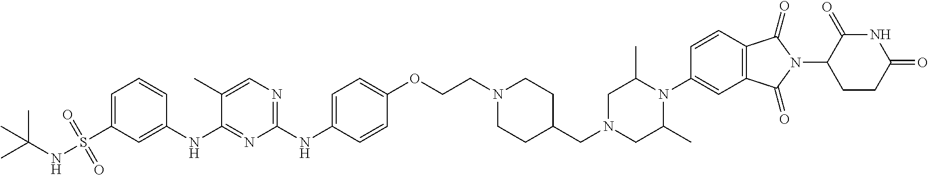 [SMILES: Cc1cnc(Nc2ccc(OCCN3CCC(CN4CC(C)N(c5ccc6c(c5)C(=O)N(C5CCC(=O)NC5=O)C6=O)C(C)C4)CC3)cc2)nc1Nc1cccc(S(=O)(=O)NC(C)(C)C)c1] | N-(tert-butyl)-3-((2-((4-(2-(4-((4-(2-(2,6- dioxopiperidin-3-yl)-1,3-dioxoisoindolin-5-yl)- 3,5-dimethylpiperazin-1-yl)methyl)piperidin-1- yl)ethoxy)phenyl)amino)-5-methylpyrimidin-4- yl)amino)benzenesulfonamide |
| | 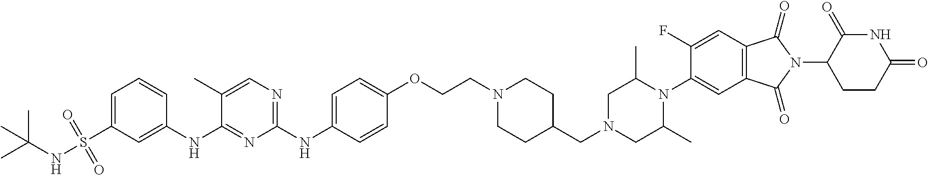 [SMILES: Cc1cnc(Nc2ccc(OCCN3CCC(CN4CC(C)N(c5cc6c(cc5F)C(=O)N(C5CCC(=O)NC5=O)C6=O)C(C)C4)CC3)cc2)nc1Nc1cccc(S(=O)(=O)NC(C)(C)C)c1] | N-(tert-butyl)-3-((2-((4-(2-(4-((4-(2-(2,6- dioxopiperidin-3-yl)-6-fluoro-1,3- dioxoisoindolin-5-yl)-3,5-dimethylpiperazin-1- yl)methyl)piperidin-1- yl)ethoxy)phenyl)amino)-5-methylpyrimidin-4- yl)amino)benzenesulfonamide |
| | 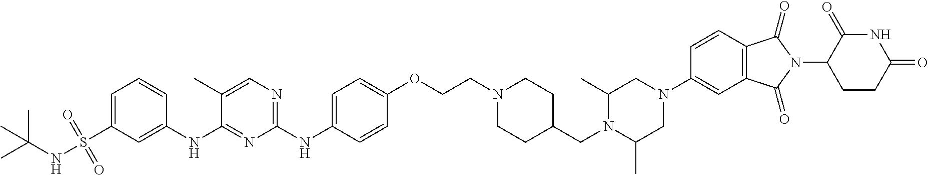 [SMILES: Cc1cnc(Nc2ccc(OCCN3CCC(CN4C(C)CN(c5ccc6c(c5)C(=O)N(C5CCC(=O)NC5=O)C6=O)CC4C)CC3)cc2)nc1Nc1cccc(S(=O)(=O)NC(C)(C)C)c1] | N-(tert-butyl)-3-((2-((4-(2-(4-((4-(2-(2,6- dioxopiperidin-3-yl)-1,3-dioxoisoindolin-5-yl)- 2,6-dimethylpiperazin-1-yl)methyl)piperidin-1- yl)ethoxy)phenyl)amino)-5-methylpyrimidin-4- yl)amino)benzenesulfonamide |
| | 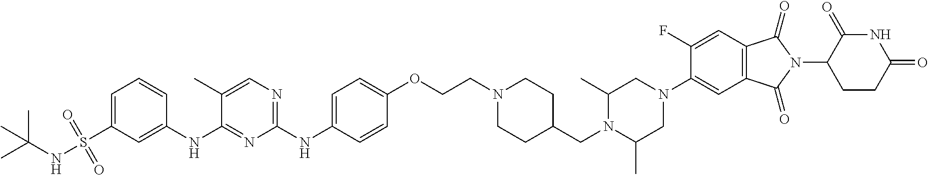 [SMILES: Cc1cnc(Nc2ccc(OCCN3CCC(CN4C(C)CN(c5cc6c(cc5F)C(=O)N(C5CCC(=O)NC5=O)C6=O)CC4C)CC3)cc2)nc1Nc1cccc(S(=O)(=O)NC(C)(C)C)c1] | N-(tert-butyl)-3-((2-((4-(2-(4-((4-(2-(2,6- dioxopiperidin-3-yl)-6-fluoro-1,3- dioxoisoindolin-5-yl)-2,6-dimethylpiperazin-1- yl)methyl)piperidin-1- yl)ethoxy)phenyl)amino)-5-methylpyrimidin-4- yl)amino)benzenesulfonamide |
| | 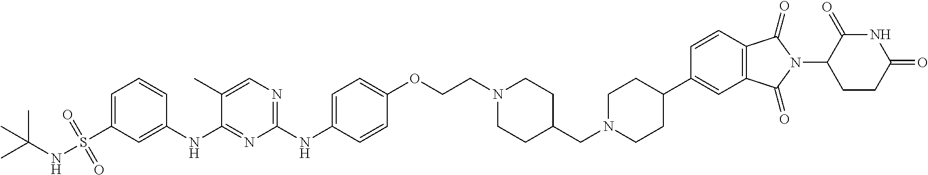 [SMILES: Cc1cnc(Nc2ccc(OCCN3CCC(CN4CCC(c5ccc6c(c5)C(=O)N(C5CCC(=O)NC5=O)C6=O)CC4)CC3)cc2)nc1Nc1cccc(S(=O)(=O)NC(C)(C)C)c1] | N-(tert-butyl)-3-((2-((4-(2-(4-((4-(2-(2,6- dioxopiperidin-3-yl)-1,3-dioxoisoindolin-5- yl)piperidin-1-yl)methyl)piperidin-1- yl)ethoxy)phenyl)amino)-5-methylpyrimidin-4- yl)amino)benzenesulfonamide |
| | 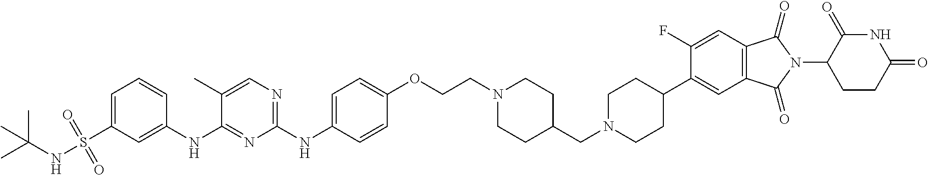 [SMILES: Cc1cnc(Nc2ccc(OCCN3CCC(CN4CCC(c5cc6c(cc5F)C(=O)N(C5CCC(=O)NC5=O)C6=O)CC4)CC3)cc2)nc1Nc1cccc(S(=O)(=O)NC(C)(C)C)c1] | N-(tert-butyl)-3-((2-((4-(2-(4-((4-(2-(2,6- dioxopiperidin-3-yl)-6-fluoro-1,3- dioxoisoindolin-5-yl)piperidin-1- yl)methyl)piperidin-1- yl)ethoxy)phenyl)amino)-5-methylpyrimidin-4- yl)amino)benzenesulfonamide |
| | 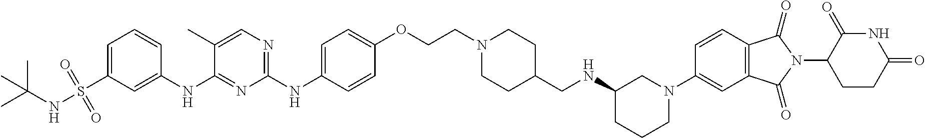 [SMILES: Cc1cnc(Nc2ccc(OCCN3CCC(CN[C@@H]4CCCN(c5ccc6c(c5)C(=O)N(C5CCC(=O)NC5=O)C6=O)C4)CC3)cc2)nc1Nc1cccc(S(=O)(=O)NC(C)(C)C)c1] | N-(tert-butyl)-3-((2-((4-(2-(4-((((3R)-1-(2-(2,6- dioxopiperidin-3-yl)-1,3-dioxoisoindolin-5- yl)piperidin-3-yl)amino)methyl)piperidin-1- yl)ethoxy)phenyl)amino)-5-methylpyrimidin-4- yl)amino)benzenesulfonamide |
| | 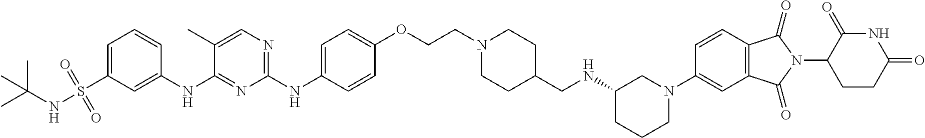 [SMILES: Cc1cnc(Nc2ccc(OCCN3CCC(CN[C@H]4CCCN(c5ccc6c(c5)C(=O)N(C5CCC(=O)NC5=O)C6=O)C4)CC3)cc2)nc1Nc1cccc(S(=O)(=O)NC(C)(C)C)c1] | N-(tert-butyl)-3-((2-((4-(2-(4-((((3S)-1-(2-(2,6- dioxopiperidin-3-yl)-1,3-dioxoisoindolin-5- yl)piperidin-3-yl)amino)methyl)piperidin-1- yl)ethoxy)phenyl)amino)-5-methylpyrimidin-4- yl)amino)benzenesulfonamide |
| | 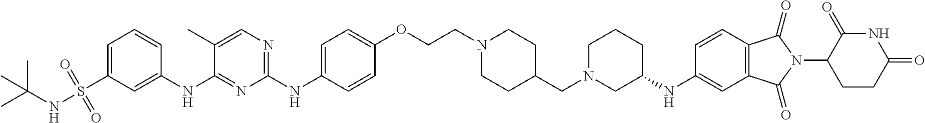 [SMILES: Cc1cnc(Nc2ccc(OCCN3CCC(CN4CCC[C@H](Nc5ccc6c(c5)C(=O)N(C5CCC(=O)NC5=O)C6=O)C4)CC3)cc2)nc1Nc1cccc(S(=O)(=O)NC(C)(C)C)c1] | N-(tert-butyl)-3-((2-((4-(2-(4-(((3S)-3-((2-(2,6- dioxopiperidin-3-yl)-1,3-dioxoisoindolin-5- yl)amino)piperidin-1-yl)methyl)piperidin-1- yl)ethoxy)phenyl)amino)-5-methylpyrimidin-4- yl)amino)benzenesulfonamide |
| | 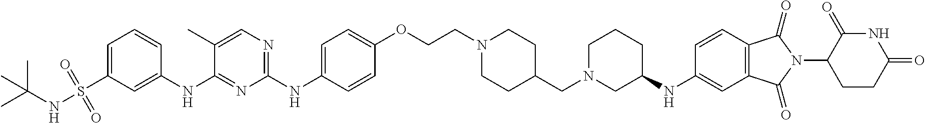 [SMILES: Cc1cnc(Nc2ccc(OCCN3CCC(CN4CCC[C@@H](Nc5ccc6c(c5)C(=O)N(C5CCC(=O)NC5=O)C6=O)C4)CC3)cc2)nc1Nc1cccc(S(=O)(=O)NC(C)(C)C)c1] | N-(tert-butyl)-3-((2-((4-(2-(4-(((3R)-3-((2-(2,6- dioxopiperidin-3-yl)-1,3-dioxoisoindolin-5- yl)amino)piperidin-1-yl)methyl)piperidin-1- yl)ethoxy)phenyl)amino)-5-methylpyrimidin-4- yl)amino)benzenesulfonamide |
| | 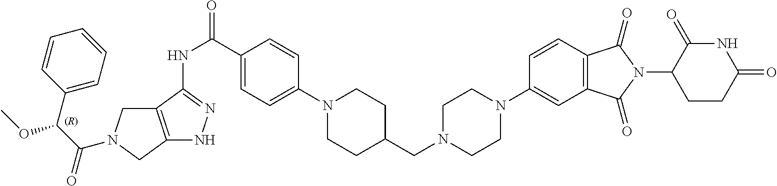 | 4-(4-((4-(2-(2,6-dioxopiperidin-3-yl)-1,3- dioxoisoindolin-5-yl)piperazin-1- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 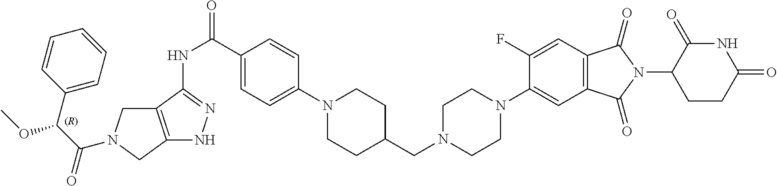 | 4-(4-((4-(2-(2,6-dioxopiperidin-3-yl)-6-fluoro- 1,3-dioxoisoindolin-5-yl)piperazin-1- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 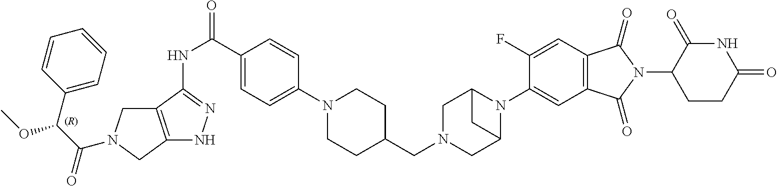 | 4-(4-((6-(2-(2,6-dioxopiperidin-3-yl)-6-fluoro- 1,3-dioxoisoindolin-5-yl)-3,6- diazabicyclo[3.1.1]heptan-3- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 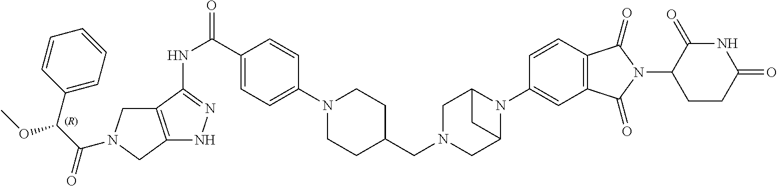 | 4-(4-((6-(2-(2,6-dioxopiperidin-3-yl)-1,3- dioxoisoindolin-5-yl)-3,6- diazabicyclo[3.1.1]heptan-3- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 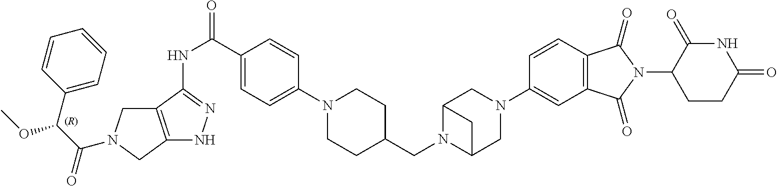 | 4-(4-((3-(2-(2,6-dioxopiperidin-3-yl)-1,3- dioxoisoindolin-5-yl)-3,6- diazabicyclo[3.1.1]heptan-6- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 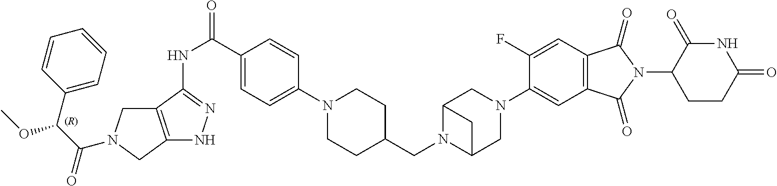 | 4-(4-((3-(2-(2,6-dioxopiperidin-3-yl)-6-fluoro- 1,3-dioxoisoindolin-5-yl)-3,6- diazabicyclo[3.1.1]heptan-6- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 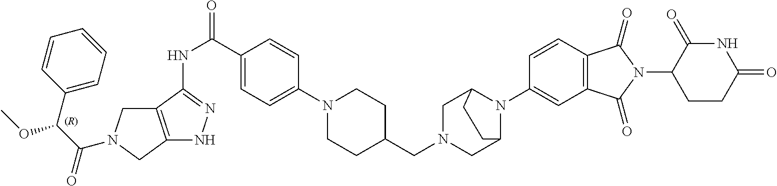 | 4-(4-((8-(2-(2,6-dioxopiperidin-3-yl)-1,3- dioxoisoindolin-5-yl)-3,8- diazabicyclo[3.2.1]octan-3-yl)methyl)piperidin- 1-yl)-N-(5-((R)-2-methoxy-2-phenylacetyl)- 1,4,5,6-tetrahydropyrrolo[3,4-c]pyrazol-3- yl)benzamide |
| | 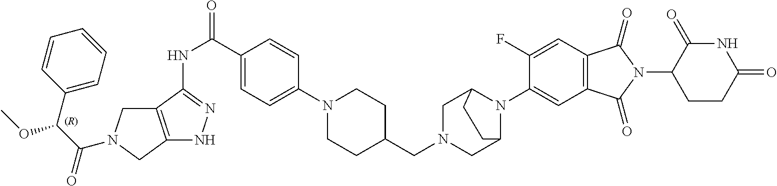 | 4-(4-((8-(2-(2,6-dioxopiperidin-3-yl)-6-fluoro- 1,3-dioxoisoindolin-5-yl)-3,8- diazabicyclo[3.2.1]octan-3-yl)methyl)piperidin- 1-yl)-N-(5-((R)-2-methoxy-2-phenylacetyl)- 1,4,5,6-tetrahydropyrrolo[3,4-c]pyrazol-3- yl)benzamide |
| | 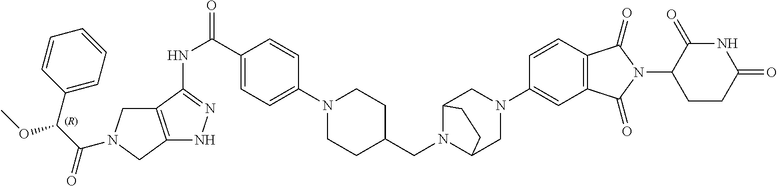 | 4-(4-((3-(2-(2,6-dioxopiperidin-3-yl)-1,3- dioxoisoindolin-5-yl)-3,8- diazabicyclo[3.2.1]octan-8-yl)methyl)piperidin- 1-yl)-N-(5-((R)-2-methoxy-2-phenylacetyl)- 1,4,5,6-tetrahydropyrrolo[3,4-c]pyrazol-3- yl)benzamide |
| | 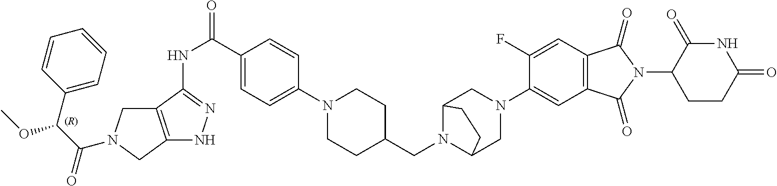 | 4-(4-((3-(2-(2,6-dioxopiperidin-3-yl)-6-fluoro- 1,3-dioxoisoindolin-5-yl)-3,8- diazabicyclo[3.2.1]octan-8-yl)methyl)piperidin- 1-yl)-N-(5-((R)-2-methoxy-2-phenylacetyl)- 1,4,5,6-tetrahydropyrrolo[3,4-c]pyrazol-3- yl)benzamide |
| | 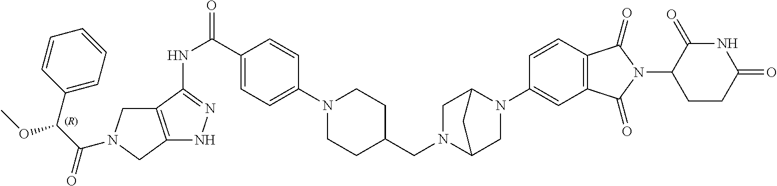 | 4-(4-((5-(2-(2,6-dioxopiperidin-3-yl)-1,3- dioxoisoindolin-5-yl)-2,5- diazabicyclo[2.2.1]heptan-2- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 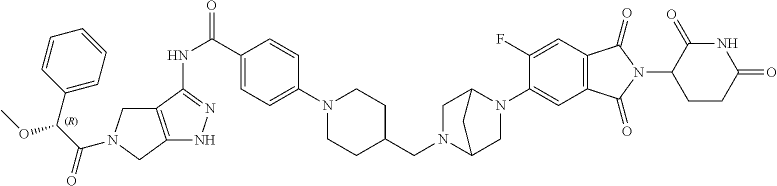 | 4-(4-((5-(2-(2,6-dioxopiperidin-3-yl)-6-fluoro- 1,3-dioxoisoindolin-5-yl)-2,5- diazabicyclo[2.2.1]heptan-2- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 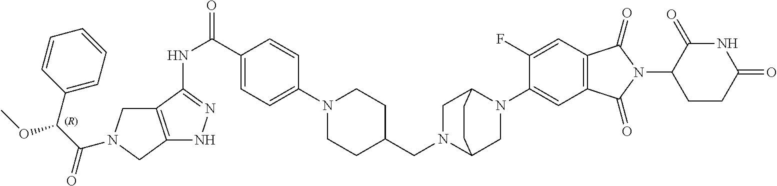 | 4-(4-((5-(2-(2,6-dioxopiperidin-3-yl)-6-fluoro- 1,3-dioxoisoindolin-5-yl)-2,5- diazabicyclo[2.2.2]octan-2-yl)methyl)piperidin- 1-yl)-N-(5-((R)-2-methoxy-2-phenylacetyl)- 1,4,5,6-tetrahydropyrrolo[3,4-c]pyrazol-3- yl)benzamide |
| | 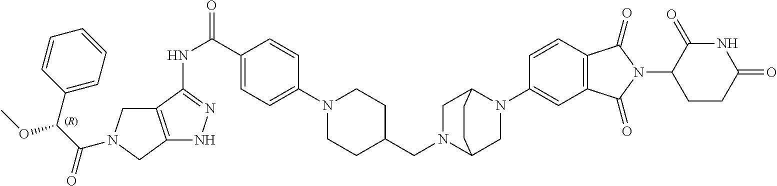 | 4-(4-((5-(2-(2,6-dioxopiperidin-3-yl)-1,3- dioxoisoindolin-5-yl)-2,5- diazabicyclo[2.2.2]octan-2-yl)methyl)piperidin- 1-yl)-N-(5-((R)-2-methoxy-2-phenylacetyl)- 1,4,5,6-tetrahydropyrrolo[3,4-c]pyrazol-3- yl)benzamide |
| | 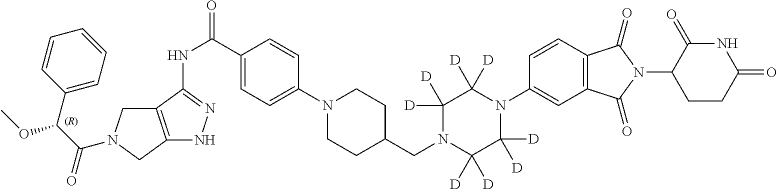 | 4-(4-((4-(2-(2,6-dioxopiperidin-3-yl)-1,3- dioxoisoindolin-5-yl)piperazin-1-yl- 2,2,3,3,5,5,6,6-d₈)methyl)piperidin-1-yl)-N-(5- ((R)-2-methoxy-2-phenylacetyl)-1,4,5,6- tetrahydropyrrolo[3,4-c]pyrazol-3- yl)benzamide |
| | 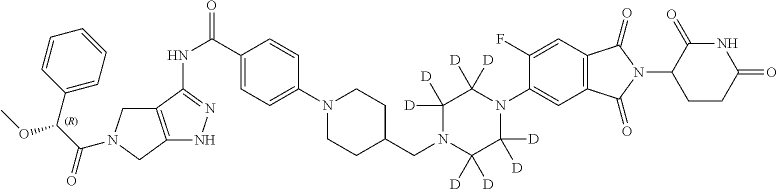 | 4-(4-((4-(2-(2,6-dioxopiperidin-3-yl)-6-fluoro- 1,3-dioxoisoindolin-5-yl)piperazin-1-yl- 2,2,3,3,5,5,6,6-d₈)methyl)piperidin-1-yl)-N-(5- ((R)-2-methoxy-2-phenylacetyl)-1,4,5,6- tetrahydropyrrolo[3,4-c]pyrazol-3- yl)benzamide |
| | 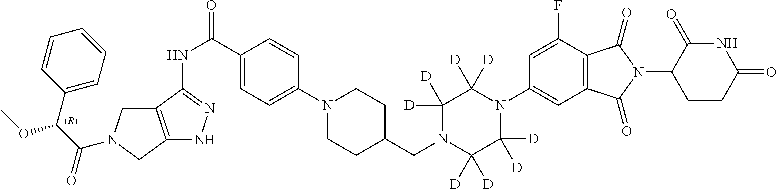 | 4-(4-((4-(2-(2,6-dioxopiperidin-3-yl)-7-fluoro- 1,3-dioxoisoindolin-5-yl)piperazin-1-yl- 2,2,3,3,5,5,6,6-d₈)methyl)piperidin-1-yl)-N-(5- ((R)-2-methoxy-2-phenylacetyl)-1,4,5,6- tetrahydropyrrolo[3,4-c]pyrazol-3- yl)benzamide |
| | 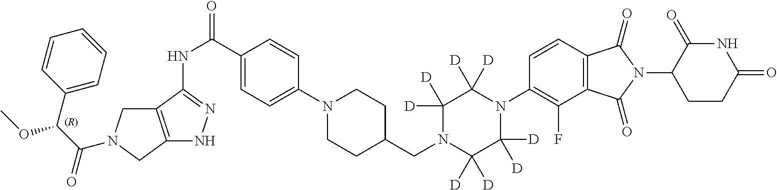 | 4-(4-((4-(2-(2,6-dioxopiperidin-3-yl)-4-fluoro- 1,3-dioxoisoindolin-5-yl)piperazin-1-yl- 2,2,3,3,5,5,6,6-d₈)methyl)piperidin-1-yl)-N-(5- ((R)-2-methoxy-2-phenylacetyl)-1,4,5,6- tetrahydropyrrolo[3,4-c]pyrazol-3- yl)benzamide |
| | 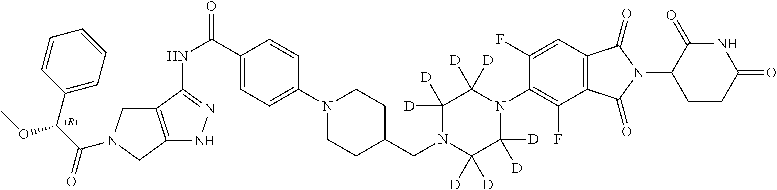 | 4-(4-((4-(2-(2,6-dioxopiperidin-3-yl)-4,6- difluoro-1,3-dioxoisoindolin-5-yl)piperazin-1- yl-2,2,3,3,5,5,6,6-d₈)methyl)piperidin-1-yl)-N- (5-((R)-2-methoxy-2-phenylacetyl)-1,4,5,6- tetrahydropyrrolo[3,4-c]pyrazol-3- yl)benzamide |
| | 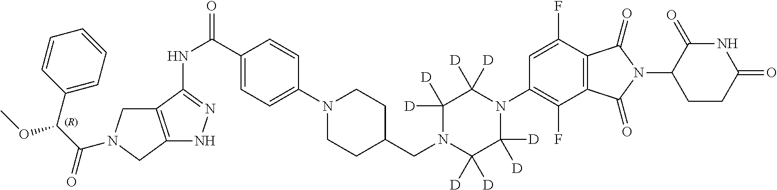 | 4-(4-((4-(2-(2,6-dioxopiperidin-3-yl)-4,7- difluoro-1,3-dioxoisoindolin-5-yl)piperazin-1- yl-2,2,3,3,5,5,6,6-d₈)methyl)piperidin-1-yl)-N- (5-((R)-2-methoxy-2-phenylacetyl)-1,4,5,6- tetrahydropyrrolo[3,4-c]pyrazol-3- yl)benzamide |
| | 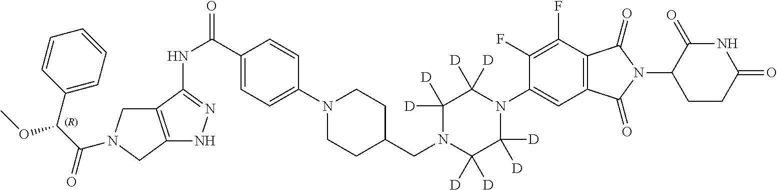 | 4-(4-((4-(2-(2,6-dioxopiperidin-3-yl)-6,7- difluoro-1,3-dioxoisoindolin-5-yl)piperazin-1- yl-2,2,3,3,5,5,6,6-d₈)methyl)piperidin-1-yl)-N- (5-((R)-2-methoxy-2-phenylacetyl)-1,4,5,6- tetrahydropyrrolo[3,4-c]pyrazol-3- yl)benzamide |
| | 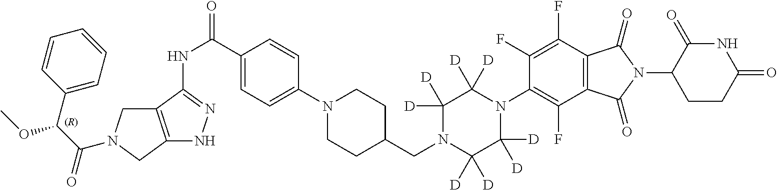 | 4-(4-((4-(2-(2,6-dioxopiperidin-3-yl)-4,6,7- trifluoro-1,3-dioxoisoindolin-5-yl)piperazin-1- yl-2,2,3,3,5,5,6,6-d₈)methyl)piperidin-1-yl)-N- (5-((R)-2-methoxy-2-phenylacetyl)-1,4,5,6- tetrahydropyrrolo[3,4-c]pyrazol-3- yl)benzamide |
| | 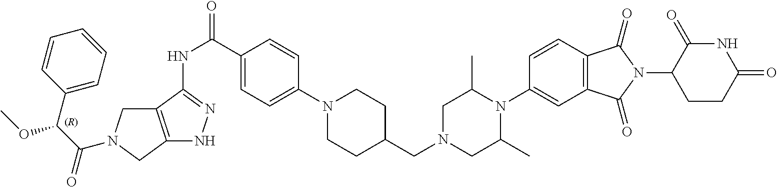 | 4-(4-((4-(2-(2,6-dioxopiperidin-3-yl)-1,3- dioxoisoindolin-5-yl)-3,5-dimethylpiperazin-1- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 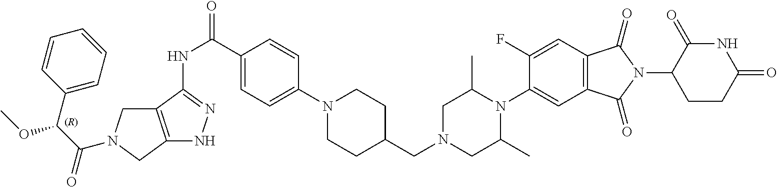 | 4-(4-((4-(2-(2,6-dioxopiperidin-3-yl)-6-fluoro- 1,3-dioxoisoindolin-5-yl)-3,5- dimethylpiperazin-1-yl)methyl)piperidin-1-yl)- N-(5-((R)-2-methoxy-2-phenylacetyl)-1,4,5,6- tetrahydropyrrolo[3,4-c]pyrazol-3- yl)benzamide |
| | 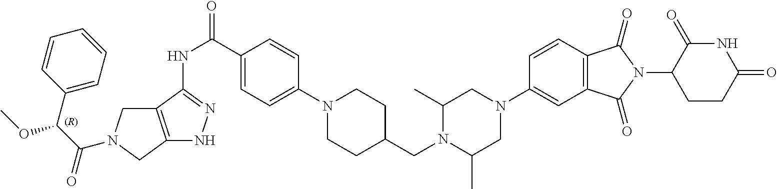 | 4-(4-((4-(2-(2,6-dioxopiperidin-3-yl)-1,3- dioxoisoindolin-5-yl)-2,6-dimethylpiperazin-1- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 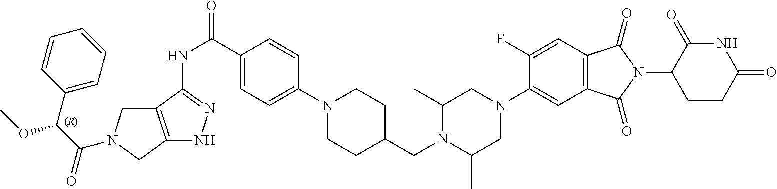 | 4-(4-((4-(2-(2,6-dioxopiperidin-3-yl)-6-fluoro- 1,3-dioxoisoindolin-5-yl)-2,6- dimethylpiperazin-1-yl)methyl)piperidin-1-yl)- N-(5-((R)-2-methoxy-2-phenylacetyl)-1,4,5,6- tetrahydropyrrolo[3,4-c]pyrazol-3- yl)benzamide |
| | 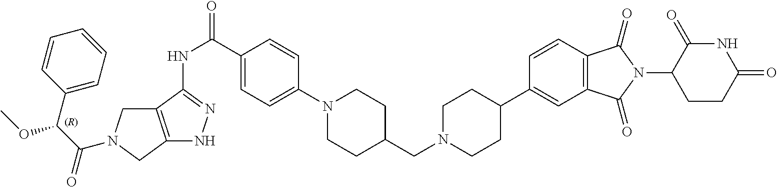 | 4-(4-((4-(2-(2,6-dioxopiperidin-3-yl)-1,3- dioxoisoindolin-5-yl)piperidin-1- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 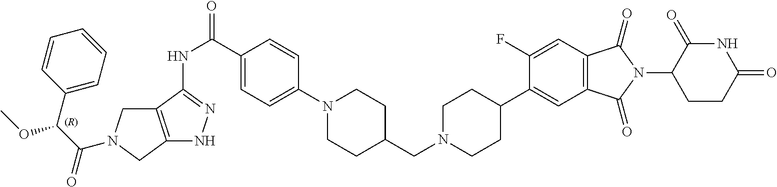 | 4-(4-((4-(2-(2,6-dioxopiperidin-3-yl)-6-fluoro- 1,3-dioxoisoindolin-5-yl)piperidin-1- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 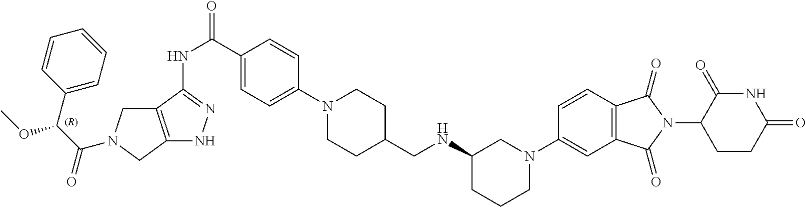 | 4-(4-((((3R)-1-(2-(2,6-dioxopiperidin-3-yl)-1,3- dioxoisoindolin-5-yl)piperidin-3- yl)amino)methyl)piperidin-1-yl)-N-(5-((R)-2- methoxy-2-phenylacetyl)-1,4,5,6- tetrahydropyrrolo[3,4-c]pyrazol-3- yl)benzamide |
| | 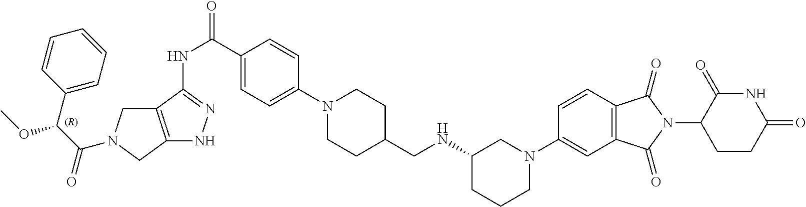 | 4-(4-((((3S)-1-(2-(2,6-dioxopiperidin-3-yl)-1,3- dioxoisoindolin-5-yl)piperidin-3- yl)amino)methyl)piperidin-1-yl)-N-(5-((R)-2- methoxy-2-phenylacetyl)-1,4,5,6- tetrahydropyrrolo[3,4-c]pyrazol-3- yl)benzamide |
| | 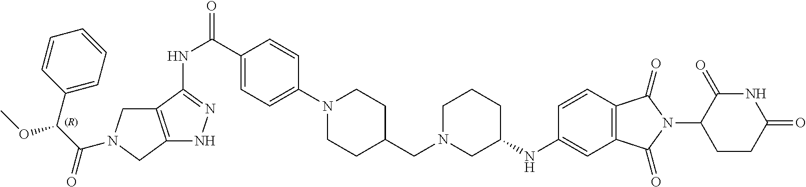 | 4-(4-(((3S)-3-((2-(2,6-dioxopiperidin-3-yl)-1,3- dioxoisoindolin-5-yl)amino)piperidin-1- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 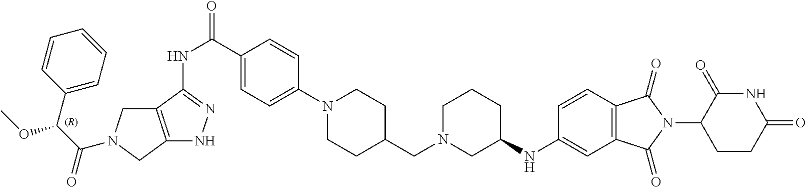 | 4-(4-(((3R)-3-((2-(2,6-dioxopiperidin-3-yl)-1,3- dioxoisoindolin-5-yl)amino)piperidin-1- yl)methyl)piperidin-1-yl)-N-(5-((R)-2- methoxy-2-phenylacetyl)-1,4,5,6- tetrahydropyrrolo[3,4-c]pyrazol-3- yl)benzamide |
| | 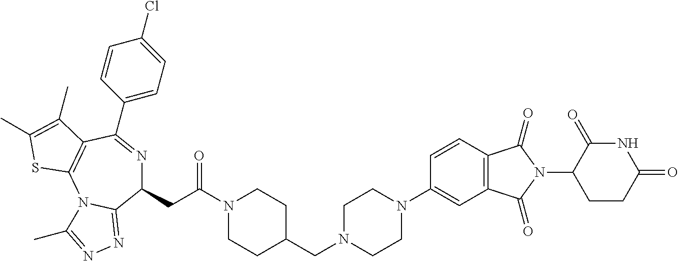 [SMILES: Cc1sc2c(c1C)C(c1ccc(Cl)cc1)=N[C@@H](CC(=O)N1CCC(CN3CCN(c4ccc5c(c4)C(=O)N(C4CCC(=O)NC4=O)C5=O)CC3)CC1)c1nnc(C)n1-2] | 5-(4-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)piperazin-1-yl)-2-(2,6- dioxopiperidin-3-yl)isoindoline-1,3-dione |
| | 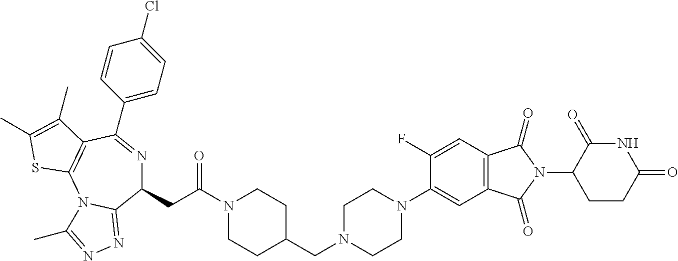 [SMILES: Cc1sc2c(c1C)C(c1ccc(Cl)cc1)=N[C@@H](CC(=O)N1CCC(CN3CCN(c4cc5c(cc4F)C(=O)N(C4CCC(=O)NC4=O)C5=O)CC3)CC1)c1nnc(C)n1-2] | 5-(4-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)piperazin-1-yl)-2-(2,6- dioxopiperidin-3-yl)-6-fluoroisoindoline-1,3- dione |
| | 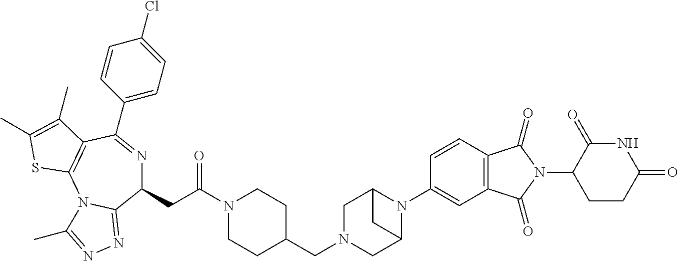 [SMILES: Cc1sc2c(c1C)C(c1ccc(Cl)cc1)=N[C@@H](CC(=O)N1CCC(CN3CC4CC(C3)N4c3ccc4c(c3)C(=O)N(C3CCC(=O)NC3=O)C4=O)CC1)c1nnc(C)n1-2] | 5-(3-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-3,6-diazabicyclo[3.1.1]heptan-6-yl)- 2-(2,6-dioxopiperidin-3-yl)isoindoline-1,3- dione |
| | 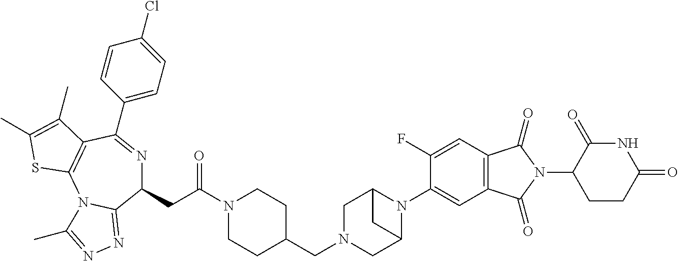 [SMILES: Cc1sc2c(c1C)C(c1ccc(Cl)cc1)=N[C@@H](CC(=O)N1CCC(CN3CC4CC(C3)N4c3cc4c(cc3F)C(=O)N(C3CCC(=O)NC3=O)C4=O)CC1)c1nnc(C)n1-2] | 5-(3-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-3,6-diazabicyclo[3.1.1]heptan-6-yl)- 2-(2,6-dioxopiperidin-3-yl)-6- fluoroisoindoline-1,3-dione |
| | 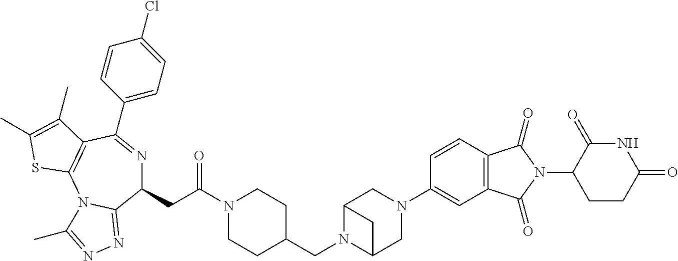 [SMILES: Cc1sc2c(c1C)C(c1ccc(Cl)cc1)=N[C@@H](CC(=O)N1CCC(CN3C4CC3CN(c3ccc5c(c3)C(=O)N(C3CCC(=O)NC3=O)C5=O)C4)CC1)c1nnc(C)n1-2] | 5-(6-((1-(2-((S)-4-(4-chlorophenyl)-2,3,9- trimethyl-6H-thieno[3,2-f] [1,2,4]triazolo [4,3- a][1,4]diazepin-6-yl)acetyl)piperidin-4- yl)methyl)-3,6-diazabicyclo[3.1.1]heptan-3-yl)- 2-(2,6-dioxopiperidin-3-yl)isoindoline-1,3- dione |
| | 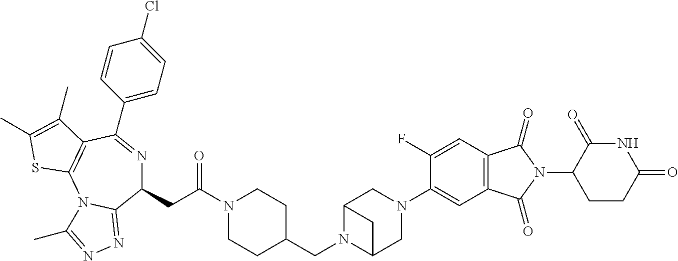 [SMILES: Cc1sc2c(c1C)C(c1ccc(Cl)cc1)=N[C@@H](CC(=O)N1CCC(CN3C4CC3CN(c3cc5c(cc3F)C(=O)N(C3CCC(=O)NC3=O)C5=O)C4)CC1)c1nnc(C)n1-2] | 5-(6-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-3,6-diazabicyclo[3.1.1]heptan-3-yl)- 2-(2,6-dioxopiperidin-3-yl)-6- fluoroisoindoline-1,3-dione |
| | 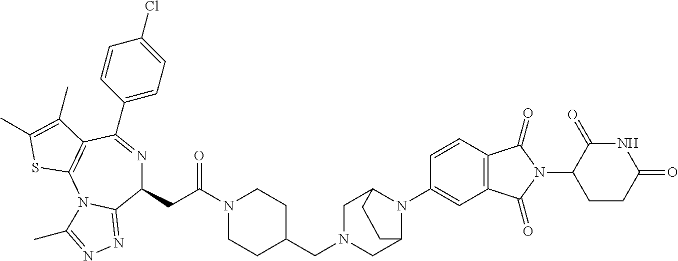 [SMILES: Cc1sc2c(c1C)C(c1ccc(Cl)cc1)=N[C@@H](CC(=O)N1CCC(CN3CC4CCC(C3)N4c3ccc4c(c3)C(=O)N(C3CCC(=O)NC3=O)C4=O)CC1)c1nnc(C)n1-2] | 5-(3-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-3,8-diazabicyclo[3.2.1]octan-8-yl)- 2-(2,6-dioxopiperidin-3-yl)isoindoline-1,3- dione |
| | 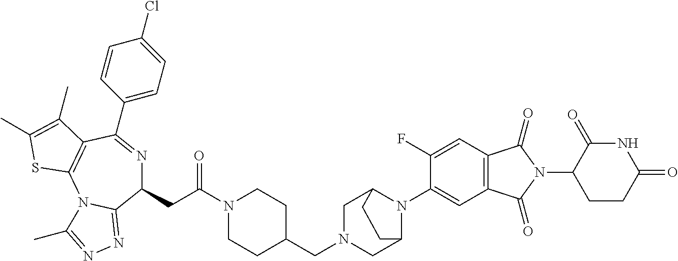 [SMILES: Cc1sc2c(c1C)C(c1ccc(Cl)cc1)=N[C@@H](CC(=O)N1CCC(CN3CC4CCC(C3)N4c3cc4c(cc3F)C(=O)N(C3CCC(=O)NC3=O)C4=O)CC1)c1nnc(C)n1-2] | 5-(3-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-3,8-diazabicyclo[3.2.1]octan-8-yl)- 2-(2,6-dioxopiperidin-3-yl)-6- fluoroisoindoline-1,3-dione |
| | 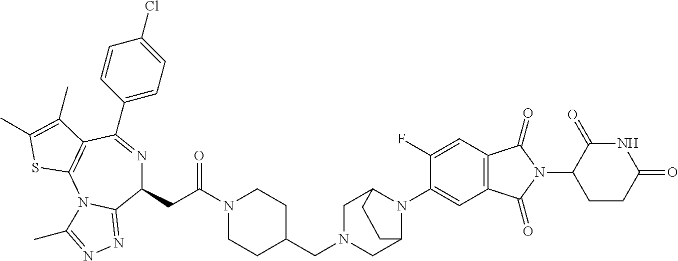 [SMILES: Cc1sc2c(c1C)C(c1ccc(Cl)cc1)=N[C@@H](CC(=O)N1CCC(CN3CC4CCC(C3)N4c3cc4c(cc3F)C(=O)N(C3CCC(=O)NC3=O)C4=O)CC1)c1nnc(C)n1-2] | 5-(3-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-3,8-diazabicyclo[3.2.1]octan-8-yl)- 2-(2,6-dioxopiperidin-3-yl)-6- fluoroisoindoline-1,3-dione |
| | 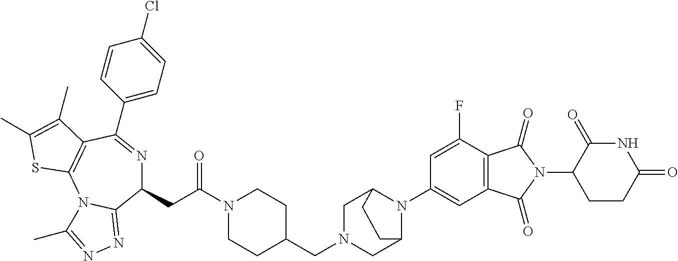 [SMILES: Cc1sc2c(c1C)C(c1ccc(Cl)cc1)=N[C@@H](CC(=O)N1CCC(CN3CC4CCC(C3)N4c3cc(F)c4c(c3)C(=O)N(C3CCC(=O)NC3=O)C4=O)CC1)c1nnc(C)n1-2] | 6-(3-((1-(2-((S)-4-(4-chlorophenyl)-2,3,9- trimethyl-6H-thieno[3,2-f] [1,2,4]triazolo [4,3- a][1,4]diazepin-6-yl)acetyl)piperidin-4- yl)methyl)-3,8-diazabicyclo[3.2.1]octan-8-yl)- 2-(2,6-dioxopiperidin-3-yl)-4- fluoroisoindoline-1,3-dione |
| | 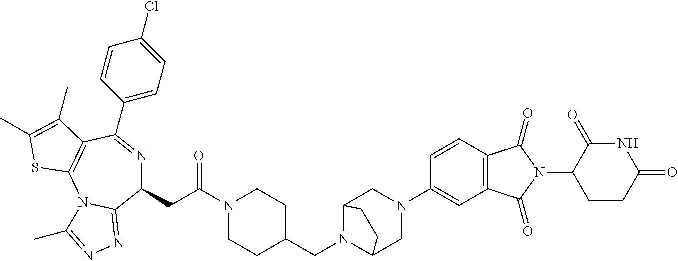 [SMILES: Cc1sc2c(c1C)C(c1ccc(Cl)cc1)=N[C@@H](CC(=O)N1CCC(CN3C4CCC3CN(c3ccc5c(c3)C(=O)N(C3CCC(=O)NC3=O)C5=O)C4)CC1)c1nnc(C)n1-2] | 5-(8-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-3,8-diazabicyclo[3.2.1]octan-3-yl)- 2-(2,6-dioxopiperidin-3-yl)isoindoline-1,3- dione |
| | 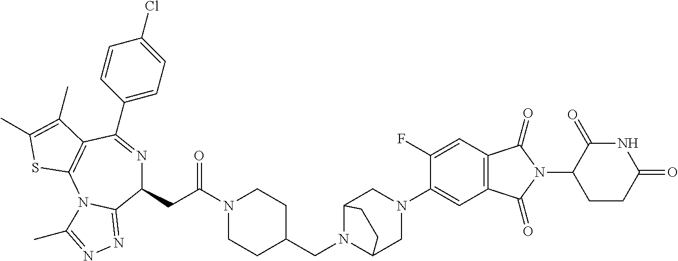 [SMILES: Cc1sc2c(c1C)C(c1ccc(Cl)cc1)=N[C@@H](CC(=O)N1CCC(CN3C4CCC3CN(c3cc5c(cc3F)C(=O)N(C3CCC(=O)NC3=O)C5=O)C4)CC1)c1nnc(C)n1-2] | 5-(8-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-3,8-diazabicyclo[3.2.1]octan-3-yl)- 2-(2,6-dioxopiperidin-3-yl)-6- fluoroisoindoline-1,3-dione |
| | 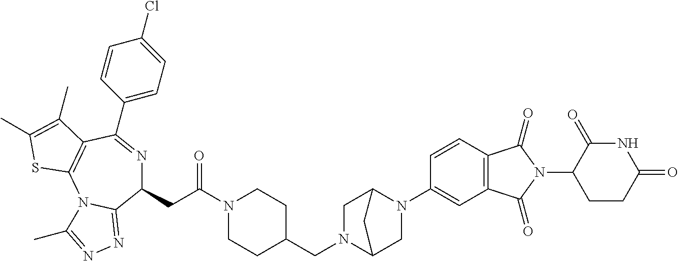 [SMILES: Cc1sc2c(c1C)C(c1ccc(Cl)cc1)=N[C@@H](CC(=O)N1CCC(CN3CC4CC3CN4c3ccc4c(c3)C(=O)N(C3CCC(=O)NC3=O)C4=O)CC1)c1nnc(C)n1-2] | 5-(5-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-2,5-diazabicyclo[2.2.1]heptan-2-yl)- 2-(2,6-dioxopiperidin-3-yl)isoindoline-1,3- dione |
| | 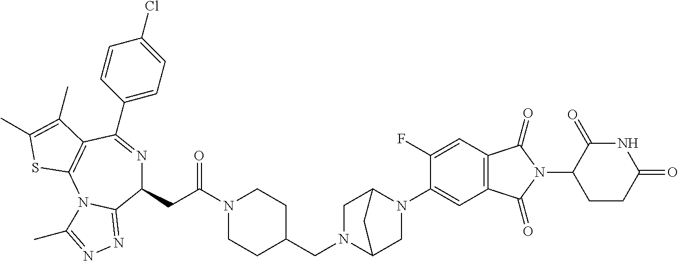 [SMILES: Cc1sc2c(c1C)C(c1ccc(Cl)cc1)=N[C@@H](CC(=O)N1CCC(CN3CC4CC3CN4c3cc4c(cc3F)C(=O)N(C3CCC(=O)NC3=O)C4=O)CC1)c1nnc(C)n1-2] | 5-(5-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-2,5-diazabicyclo[2.2.1]heptan-2-yl)- 2-(2,6-dioxopiperidin-3-yl)-6- fluoroisoindoline-1,3-dione |
| | 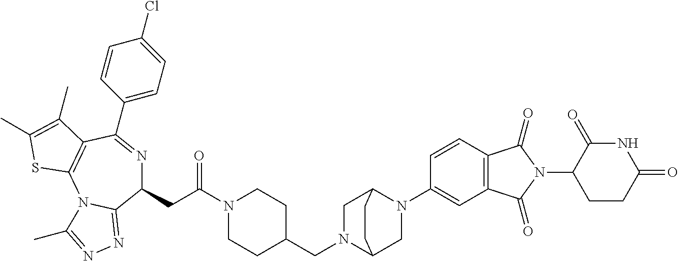 [SMILES: Cc1sc2c(c1C)C(c1ccc(Cl)cc1)=N[C@@H](CC(=O)N1CCC(CN3CC4CCC3CN4c3ccc4c(c3)C(=O)N(C3CCC(=O)NC3=O)C4=O)CC1)c1nnc(C)n1-2] | 5-(5-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-2,5-diazabicyclo[2.2.2]octan-2-yl)- 2-(2,6-dioxopiperidin-3-yl)isoindoline-1,3- dione |
| | 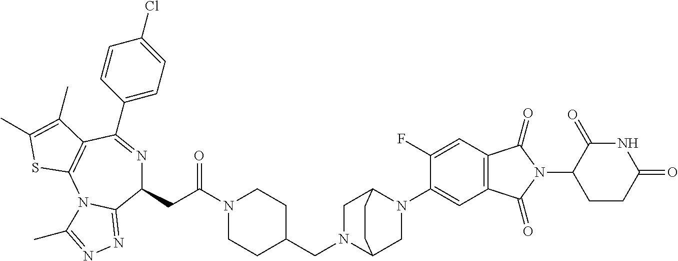 [SMILES: Cc1sc2c(c1C)C(c1ccc(Cl)cc1)=N[C@@H](CC(=O)N1CCC(CN3CC4CCC3CN4c3cc4c(cc3F)C(=O)N(C3CCC(=O)NC3=O)C4=O)CC1)c1nnc(C)n1-2] | 5-(5-((1-(2-((S)-4-(4-chlorophenyl)-2,3,9- trimethyl-6H-thieno[3,2-f] [1,2,4]triazolo [4,3- a][1,4]diazepin-6-yl)acetyl)piperidin-4- yl)methyl)-2,5-diazabicyclo[2.2.2]octan-2-yl)- 2-(2,6-dioxopiperidin-3-yl)-6- fluoroisoindoline-1,3-dione |
| | 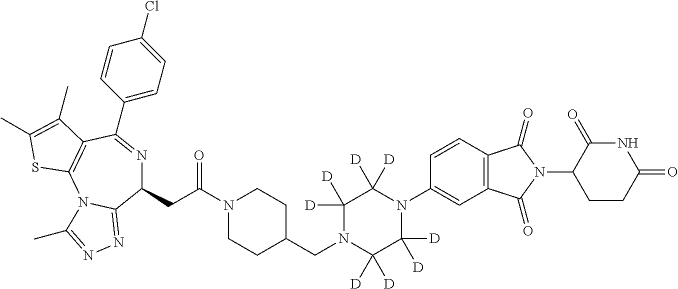 [SMILES: [2H]C1([2H])N(CC2CCN(C(=O)C[C@@H]3N=C(c4ccc(Cl)cc4)c4c(sc(C)c4C)-n4c(C)nnc43)CC2)C([2H])([2H])C([2H])([2H])N(c2ccc3c(c2)C(=O)N(C2CCC(=O)NC2=O)C3=O)C1([2H])[2H]] | 5-(4-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)piperazin-1-yl-2,2,3,3,5,5,6,6-d₈)-2- (2,6-dioxopiperidin-3-yl)isoindoline-1,3-dione |
| | 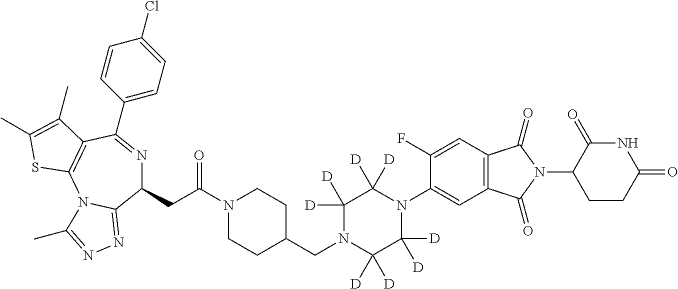 [SMILES: [2H]C1([2H])N(CC2CCN(C(=O)C[C@@H]3N=C(c4ccc(Cl)cc4)c4c(sc(C)c4C)-n4c(C)nnc43)CC2)C([2H])([2H])C([2H])([2H])N(c2cc3c(cc2F)C(=O)N(C2CCC(=O)NC2=O)C3=O)C1([2H])[2H]] | 5-(4-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)piperazin-1-yl-2,2,3,3,5,5,6,6-d₈)-2- (2,6-dioxopiperidin-3-yl)-6-fluoroisoindoline- 1,3-dione |
| | 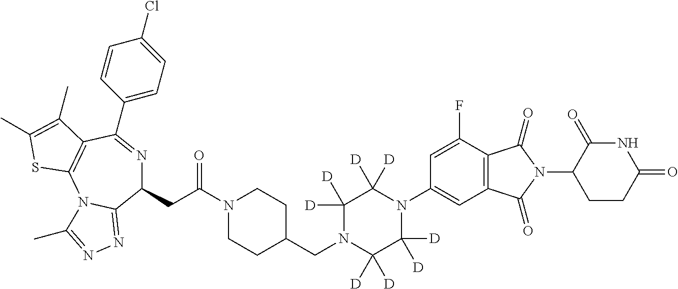 [SMILES: [2H]C1([2H])N(CC2CCN(C(=O)C[C@@H]3N=C(c4ccc(Cl)cc4)c4c(sc(C)c4C)-n4c(C)nnc43)CC2)C([2H])([2H])C([2H])([2H])N(c2cc(F)c3c(c2)C(=O)N(C2CCC(=O)NC2=O)C3=O)C1([2H])[2H]] | 6-(4-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 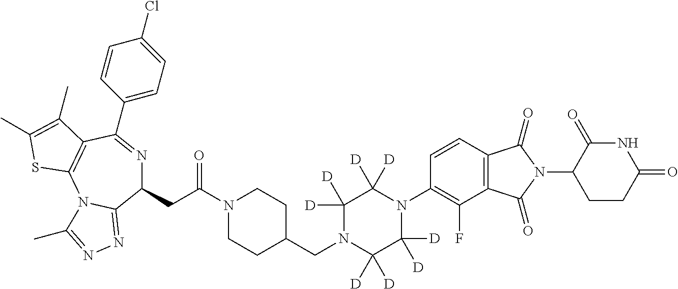 [SMILES: [2H]C1([2H])N(CC2CCN(C(=O)C[C@@H]3N=C(c4ccc(Cl)cc4)c4c(sc(C)c4C)-n4c(C)nnc43)CC2)C([2H])([2H])C([2H])([2H])N(c2ccc3c(c2F)C(=O)N(C2CCC(=O)NC2=O)C3=O)C1([2H])[2H]] | 5-(4-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 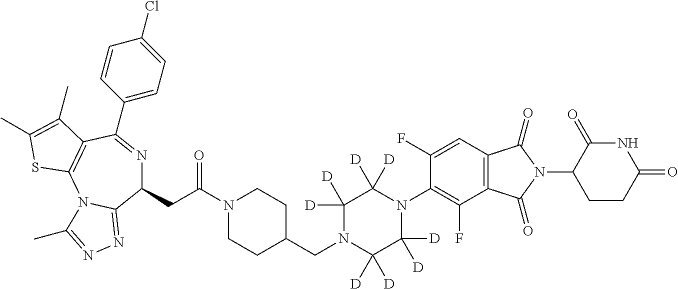 [SMILES: [2H]C1([2H])N(CC2CCN(C(=O)C[C@@H]3N=C(c4ccc(Cl)cc4)c4c(sc(C)c4C)-n4c(C)nnc43)CC2)C([2H])([2H])C([2H])([2H])N(c2c(F)cc3c(c2F)C(=O)N(C2CCC(=O)NC2=O)C3=O)C1([2H])[2H]] | 5-(4-((1-(2-((S)-4-(4-chlorophenyl)-2,3,9- trimethyl-6H-thieno[3,2-f][1,2,4]triazolo[4,3- a][1,4]diazepin-6-yl)acetyl)piperidin-4- yl)methyl)piperazin-1-yl-2,2,3,3,5,5,6,6-d₈)-2- (2,6-dioxopiperidin-3-yl)-4,6- difluoroisoindoline-1,3-dione |
| | 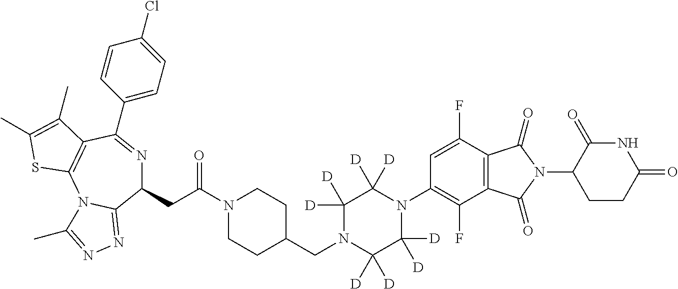 [SMILES: [2H]C1([2H])N(CC2CCN(C(=O)C[C@@H]3N=C(c4ccc(Cl)cc4)c4c(sc(C)c4C)-n4c(C)nnc43)CC2)C([2H])([2H])C([2H])([2H])N(c2cc(F)c3c(c2F)C(=O)N(C2CCC(=O)NC2=O)C3=O)C1([2H])[2H]] | 5-(4-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)piperazin-1-yl-2,2,3,3,5,5,6,6-d₈)-2- (2,6-dioxopiperidin-3-yl)-4,7- difluoroisoindoline-1,3-dione |
| | 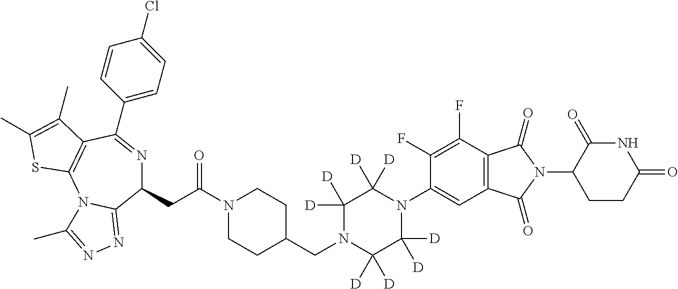 [SMILES: [2H]C1([2H])N(CC2CCN(C(=O)C[C@@H]3N=C(c4ccc(Cl)cc4)c4c(sc(C)c4C)-n4c(C)nnc43)CC2)C([2H])([2H])C([2H])([2H])N(c2cc3c(c(F)c2F)C(=O)N(C2CCC(=O)NC2=O)C3=O)C1([2H])[2H]] | 6-(4-((1-(2-((S)-4-(4-chlorophenyl)-2,3,9- trimethyl-6H-thieno[3,2-f] [1,2,4]triazolo [4,3- a][1,4]diazepin-6-yl)acetyl)piperidin-4- yl)methyl)piperazin-1-yl-2,2,3,3,5,5,6,6-d₈)-2- (2,6-dioxopiperidin-3-yl)-4,5- difluoroisoindoline-1,3-dione |
| | 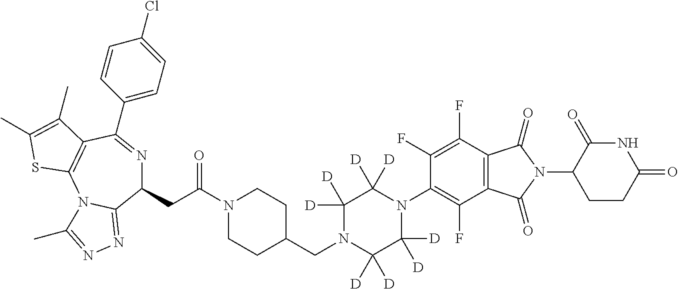 [SMILES: [2H]C1([2H])N(CC2CCN(C(=O)C[C@@H]3N=C(c4ccc(Cl)cc4)c4c(sc(C)c4C)-n4c(C)nnc43)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)piperazin-1-yl-2,2,3,3,5,5,6,6-d₈)-2- (2,6-dioxopiperidin-3-yl)-4,6,7- trifluoroisoindoline-1,3-dione |
| | 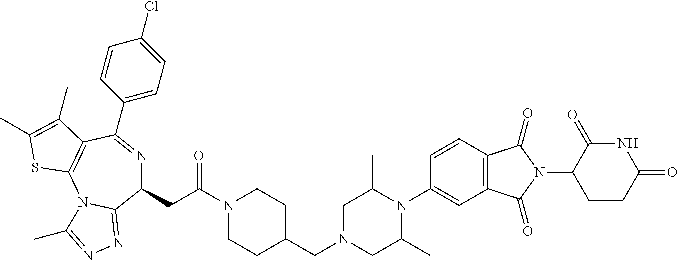 [SMILES: Cc1sc2c(c1C)C(c1ccc(Cl)cc1)=N[C@@H](CC(=O)N1CCC(CN3CC(C)N(c4ccc5c(c4)C(=O)N(C4CCC(=O)NC4=O)C5=O)C(C)C3)CC1)c1nnc(C)n1-2] | 5-(4-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-2,6-dimethylpiperazin-1-yl)-2-(2,6- dioxopiperidin-3-yl)isoindoline-1,3-dione |
| | 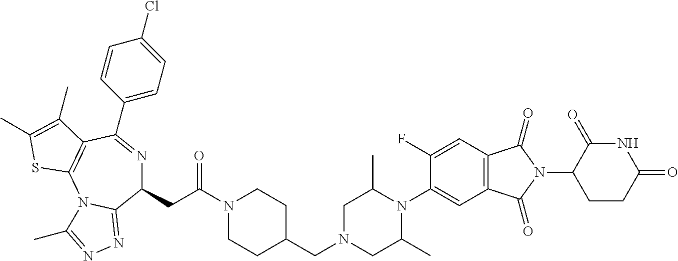 [SMILES: Cc1sc2c(c1C)C(c1ccc(Cl)cc1)=N[C@@H](CC(=O)N1CCC(CN3CC(C)N(c4cc5c(cc4F)C(=O)N(C4CCC(=O)NC4=O)C5=O)C(C)C3)CC1)c1nnc(C)n1-2] | 5-(4-((1-(2-((S)-4-(4-chlorophenyl)-2,3,9- trimethyl-6H-thieno[3,2-f] [1,2,4]triazolo [4,3- a][1,4]diazepin-6-yl)acetyl)piperidin-4- yl)methyl)-2,6-dimethylpiperazin-1-yl)-2-(2,6- dioxopiperidin-3-yl)-6-fluoroisoindoline-1,3- dione |
| | 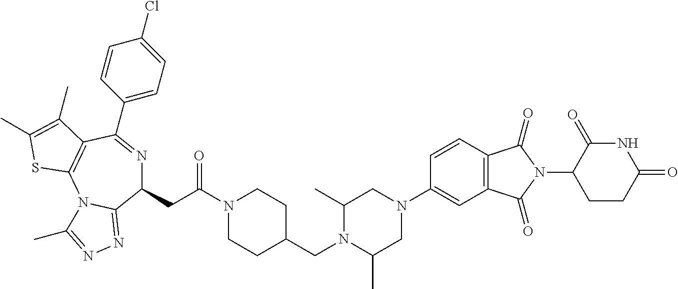 [SMILES: Cc1sc2c(c1C)C(c1ccc(Cl)cc1)=N[C@@H](CC(=O)N1CCC(CN3C(C)CN(c4ccc5c(c4)C(=O)N(C4CCC(=O)NC4=O)C5=O)CC3C)CC1)c1nnc(C)n1-2] | 5-(4-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-3,5-dimethylpiperazin-1-yl)-2-(2,6- dioxopiperidin-3-yl)isoindoline-1,3-dione |
| | 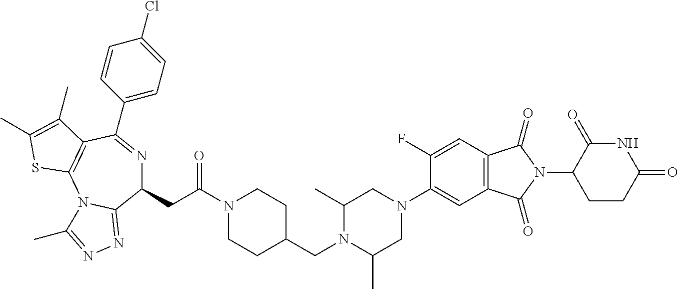 [SMILES: Cc1sc2c(c1C)C(c1ccc(Cl)cc1)=N[C@@H](CC(=O)N1CCC(CN3C(C)CN(c4cc5c(cc4F)C(=O)N(C4CCC(=O)NC4=O)C5=O)CC3C)CC1)c1nnc(C)n1-2] | 5-(4-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)-3,5-dimethylpiperazin-1-yl)-2-(2,6- dioxopiperidin-3-yl)-6-fluoroisoindoline-1,3- dione |
| | 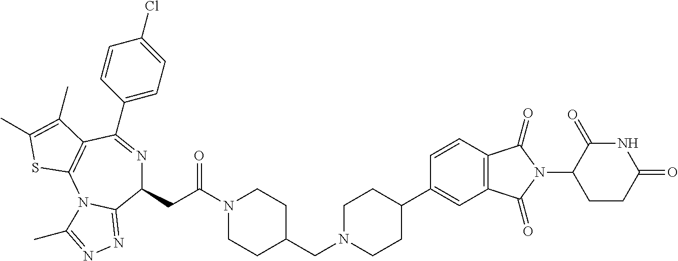 [SMILES: Cc1sc2c(c1C)C(c1ccc(Cl)cc1)=N[C@@H](CC(=O)N1CCC(CN3CCC(c4ccc5c(c4)C(=O)N(C4CCC(=O)NC4=O)C5=O)CC3)CC1)c1nnc(C)n1-2] | 5-(1-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)piperidin-4-yl)-2-(2,6- dioxopiperidin-3-yl)isoindoline-1,3-dione |
| | 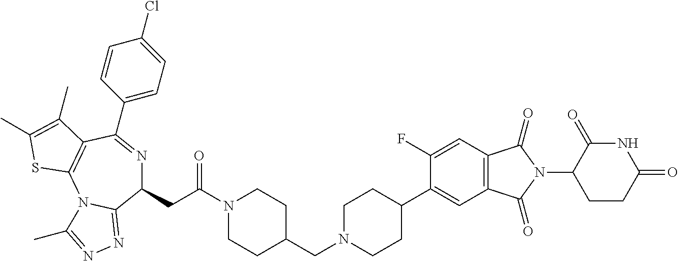 [SMILES: Cc1sc2c(c1C)C(c1ccc(Cl)cc1)=N[C@@H](CC(=O)N1CCC(CN3CCC(c4cc5c(cc4F)C(=O)N(C4CCC(=O)NC4=O)C5=O)CC3)CC1)c1nnc(C)n1-2] | 5-(1-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)piperidin-4-yl)-2-(2,6- dioxopiperidin-3-yl)-6-fluoroisoindoline-1,3- dione |
| | 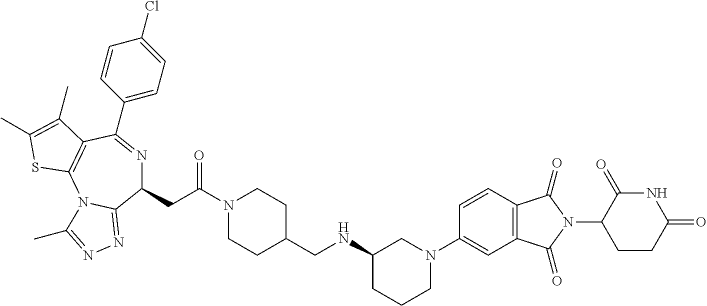 [SMILES: Cc1sc2c(c1C)C(c1ccc(Cl)cc1)=N[C@@H](CC(=O)N1CCC(CN[C@@H]3CCCN(c4ccc5c(c4)C(=O)N(C4CCC(=O)NC4=O)C5=O)C3)CC1)c1nnc(C)n1-2] | 5-((R)-3-(((1-(2-((S)-4-(4-chlorophenyl)-2,3,9- trimethyl-6H-thieno[3,2-f] [1,2,4]triazolo [4,3- a][1,4]diazepin-6-yl)acetyl)piperidin-4- yl)methyl)amino)piperidin-1-yl)-2-(2,6- dioxopiperidin-3-yl)isoindoline-1,3-dione |
| | 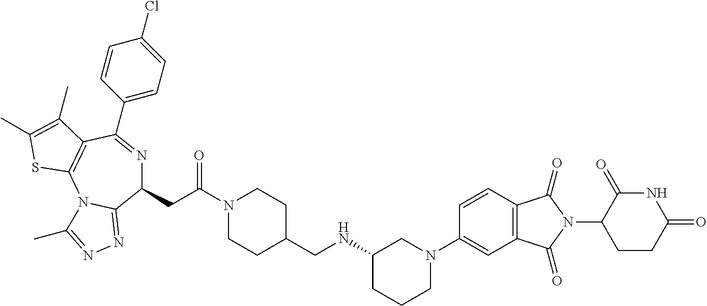 [SMILES: Cc1sc2c(c1C)C(c1ccc(Cl)cc1)=N[C@@H](CC(=O)N1CCC(CN[C@H]3CCCN(c4ccc5c(c4)C(=O)N(C4CCC(=O)NC4=O)C5=O)C3)CC1)c1nnc(C)n1-2] | 5-((S)-3-(((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)amino)piperidin-1-yl)-2-(2,6- dioxopiperidin-3-yl)isoindoline-1,3-dione |
| | 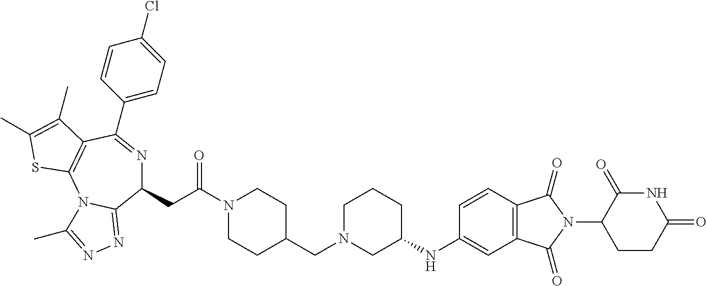 [SMILES: Cc1sc2c(c1C)C(c1ccc(Cl)cc1)=N[C@@H](CC(=O)N1CCC(CN3CCC[C@H](Nc4ccc5c(c4)C(=O)N(C4CCC(=O)NC4=O)C5=O)C3)CC1)c1nnc(C)n1-2] | 5-(((S)-1-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)piperidin-3-yl)amino)-2-(2,6- dioxopiperidin-3-yl)isoindoline-1,3-dione |
| | 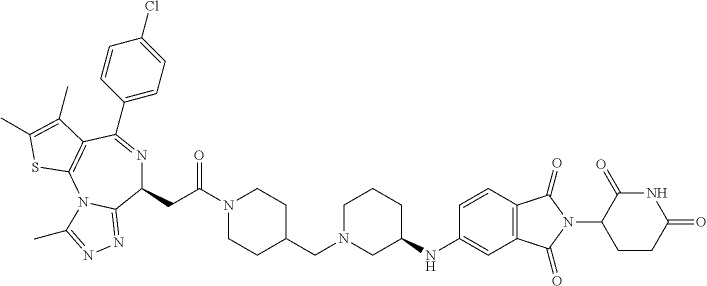 [SMILES: Cc1sc2c(c1C)C(c1ccc(Cl)cc1)=N[C@@H](CC(=O)N1CCC(CN3CCC[C@@H](Nc4ccc5c(c4)C(=O)N(C4CCC(=O)NC4=O)C5=O)C3)CC1)c1nnc(C)n1-2] | 5-(((R)-1-((1-(2-((S)-4-(4-chlorophenyl)-2,3,9- trimethyl-6H-thieno[3,2-f] [1,2,4]triazolo[4,3- a][1,4]diazepin-6-yl)acetyl)piperidin-4- yl)methyl)piperidin-3-yl)amino)-2-(2,6- dioxopiperidin-3-yl)isoindoline-1,3-dione |
| | 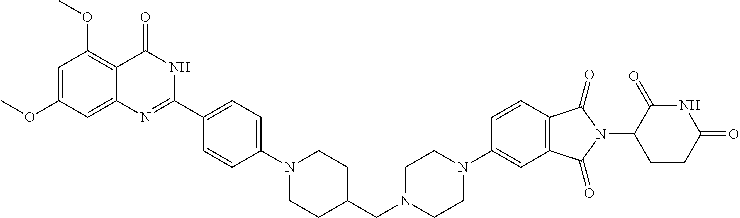 [SMILES: COc1cc(OC)c2c(=O)[nH]c(-c3ccc(N4CCC(CN5CCN(c6ccc7c(c6)C(=O)N(C6CCC(=O)NC6=O)C7=O)CC5)CC4)cc3)nc2c1] | 5-(4-((1-(4-(5,7-dimethoxy-4-oxo-3,4- dihydroquinazolin-2-yl)phenyl)piperidin-4- yl)methyl)piperazin-1-yl)-2-(2,6- dioxopiperidin-3-yl)isoindoline-1,3-dione |
| | 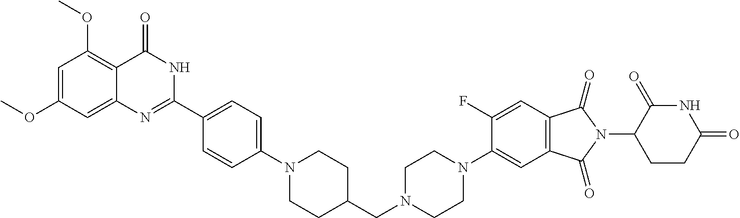 [SMILES: COc1cc(OC)c2c(=O)[nH]c(-c3ccc(N4CCC(CN5CCN(c6cc7c(cc6F)C(=O)N(C6CCC(=O)NC6=O)C7=O)CC5)CC4)cc3)nc2c1] | 5-(4-((1-(4-(5,7-dimethoxy-4-oxo-3,4- dihydroquinazolin-2-yl)phenyl)piperidin-4- yl)methyl)piperazin-1-yl)-2-(2,6- dioxopiperidin-3-yl)-6-fluoroisoindoline-1,3- dione |
| | 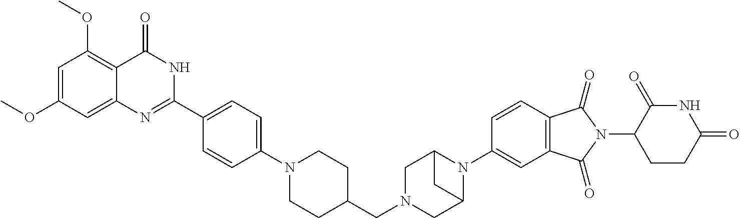 [SMILES: COc1cc(OC)c2c(=O)[nH]c(-c3ccc(N4CCC(CN5CC6CC(C5)N6c5ccc6c(c5)C(=O)N(C5CCC(=O)NC5=O)C6=O)CC4)cc3)nc2c1] | 5-(3-((1-(4-(5,7-dimethoxy-4-oxo-3,4- dihydroquinazolin-2-yl)phenyl)piperidin-4- yl)methyl)-3,6-diazabicyclo[3.1.1]heptan-6-yl)- 2-(2,6-dioxopiperidin-3-yl)isoindoline-1,3- dione |
| | 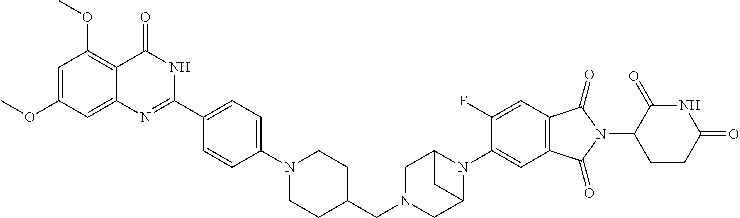 [SMILES: COc1cc(OC)c2c(=O)[nH]c(-c3ccc(N4CCC(CN5CC6CC(C5)N6c5cc6c(cc5F)C(=O)N(C5CCC(=O)NC5=O)C6=O)CC4)cc3)nc2c1] | 5-(3-((1-(4-(5,7-dimethoxy-4-oxo-3,4- dihydroquinazolin-2-yl)phenyl)piperidin-4- yl)methyl)-3,6-diazabicyclo[3.1.1]heptan-6-yl)- 2-(2,6-dioxopiperidin-3-yl)-6- fluoroisoindoline-1,3-dione |
| | 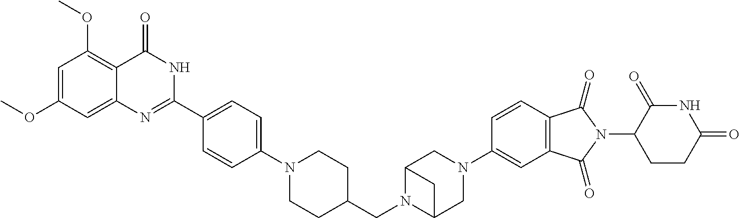 [SMILES: COc1cc(OC)c2c(=O)[nH]c(-c3ccc(N4CCC(CN5C6CC5CN(c5ccc7c(c5)C(=O)N(C5CCC(=O)NC5=O)C7=O)C6)CC4)cc3)nc2c1] | 5-(6-((1-(4-(5,7-dimethoxy-4-oxo-3,4- dihydroquinazolin-2-yl)phenyl)piperidin-4- yl)methyl)-3,6-diazabicyclo[3.1.1]heptan-3-yl)- 2-(2,6-dioxopiperidin-3-yl)isoindoline-1,3- dione |
| | 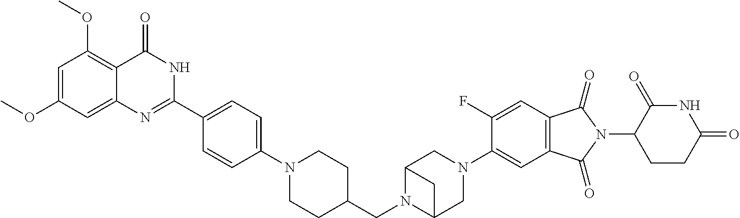 [SMILES: COc1cc(OC)c2c(=O)[nH]c(-c3ccc(N4CCC(CN5C6CC5CN(c5cc7c(cc5F)C(=O)N(C5CCC(=O)NC5=O)C7=O)C6)CC4)cc3)nc2c1] | 5-(6-((1-(4-(5,7-dimethoxy-4-oxo-3,4- dihydroquinazolin-2-yl)phenyl)piperidin-4- yl)methyl)-3,6-diazabicyclo[3.1.1]heptan-3-yl)- 2-(2,6-dioxopiperidin-3-yl)-6- fluoroisoindoline-1,3-dione |
| | 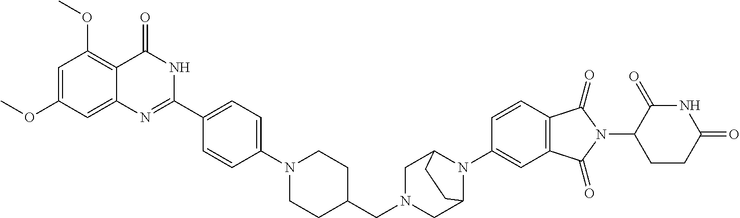 [SMILES: COc1cc(OC)c2c(=O)[nH]c(-c3ccc(N4CCC(CN5CC6CCC(C5)N6c5ccc6c(c5)C(=O)N(C5CCC(=O)NC5=O)C6=O)CC4)cc3)nc2c1] | 5-(3-((1-(4-(5,7-dimethoxy-4-oxo-3,4- dihydroquinazolin-2-yl)phenyl)piperidin-4- yl)methyl)-3,8-diazabicyclo[3.2.1]octan-8-yl)- 2-(2,6-dioxopiperidin-3-yl)isoindoline-1,3- dione |
| | 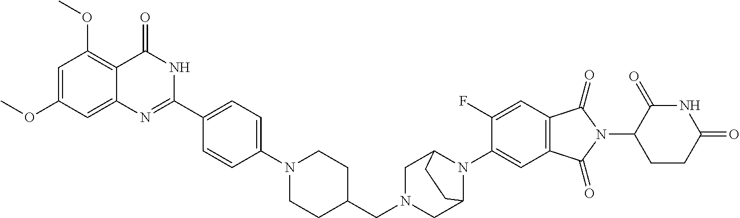 [SMILES: COc1cc(OC)c2c(=O)[nH]c(-c3ccc(N4CCC(CN5CC6CCC(C5)N6c5cc6c(cc5F)C(=O)N(C5CCC(=O)NC5=O)C6=O)CC4)cc3)nc2c1] | 5-(3-((1-(4-(5,7-dimethoxy-4-oxo-3,4- dihydroquinazolin-2-yl)phenyl)piperidin-4- yl)methyl)-3,8-diazabicyclo[3.2.1]octan-8-yl)- 2-(2,6-dioxopiperidin-3-yl)isoindoline-1,3- dione |
| | 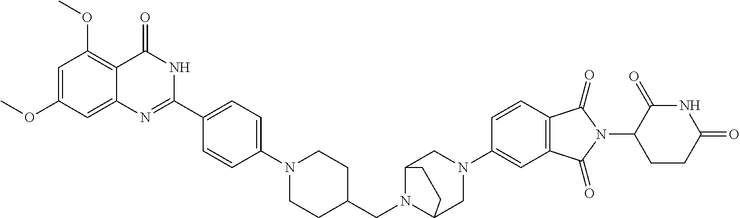 [SMILES: COc1cc(OC)c2c(=O)[nH]c(-c3ccc(N4CCC(CN5C6CCC5CN(c5ccc7c(c5)C(=O)N(C5CCC(=O)NC5=O)C7=O)C6)CC4)cc3)nc2c1] | 5-(8-((1-(4-(5,7-dimethoxy-4-oxo-3,4- dihydroquinazolin-2-yl)phenyl)piperidin-4- yl)methyl)-3,8-diazabicyclo[3.2.1]octan-3-yl)- 2-(2,6-dioxopiperidin-3-yl)isoindoline-1,3- dione |
| | 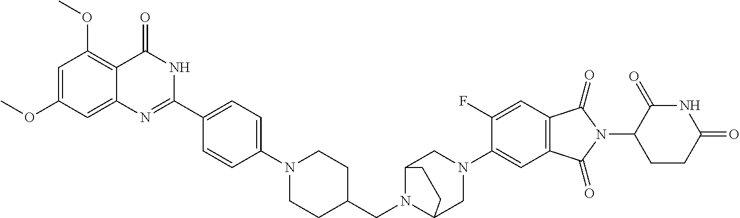 [SMILES: COc1cc(OC)c2c(=O)[nH]c(-c3ccc(N4CCC(CN5C6CCC5CN(c5cc7c(cc5F)C(=O)N(C5CCC(=O)NC5=O)C7=O)C6)CC4)cc3)nc2c1] | 5-(8-((1-(4-(5,7-dimethoxy-4-oxo-3,4- dihydroquinazolin-2-yl)phenyl)piperidin-4- yl)methyl)-3,8-diazabicyclo[3.2.1]octan-3-yl)- 2-(2,6-dioxopiperidin-3-yl)-6- fluoroisoindoline-1,3-dione |
| | 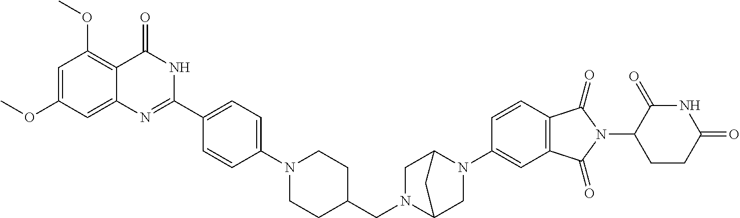 [SMILES: COc1cc(OC)c2c(=O)[nH]c(-c3ccc(N4CCC(CN5CC6CC5CN6c5ccc6c(c5)C(=O)N(C5CCC(=O)NC5=O)C6=O)CC4)cc3)nc2c1] | 5-(5-((1-(4-(5,7-dimethoxy-4-oxo-3,4- dihydroquinazolin-2-yl)phenyl)piperidin-4- yl)methyl)-2,5-diazabicyclo[2.2.1]heptan-2-yl)- 2-(2,6-dioxopiperidin-3-yl)isoindoline-1,3- dione |
| | 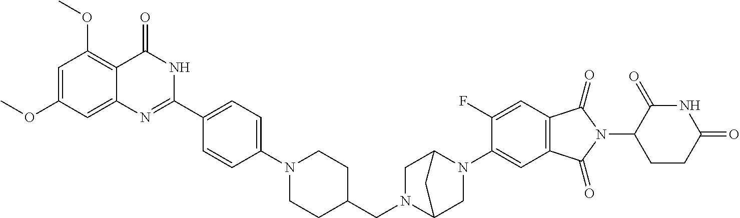 [SMILES: COc1cc(OC)c2c(=O)[nH]c(-c3ccc(N4CCC(CN5CC6CC5CN6c5cc6c(cc5F)C(=O)N(C5CCC(=O)NC5=O)C6=O)CC4)cc3)nc2c1] | 5-(5-((1-(4-(5,7-dimethoxy-4-oxo-3,4- dihydroquinazolin-2-yl)phenyl)piperidin-4- yl)methyl)-2,5-diazabicyclo[2.2.1]heptan-2-yl)- 2-(2,6-dioxopiperidin-3-yl)-6- fluoroisoindoline-1,3-dione |
| | 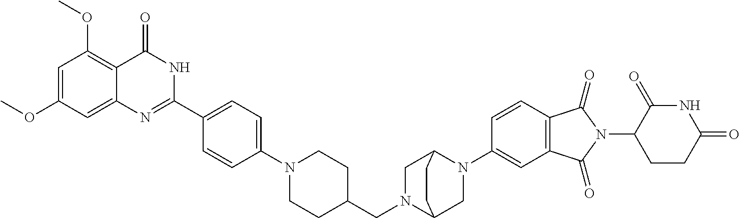 [SMILES: COc1cc(OC)c2c(=O)[nH]c(-c3ccc(N4CCC(CN5CC6CCC5CN6c5ccc6c(c5)C(=O)N(C5CCC(=O)NC5=O)C6=O)CC4)cc3)nc2c1] | 5-(5-((1-(4-(5,7-dimethoxy-4-oxo-3,4- dihydroquinazolin-2-yl)phenyl)piperidin-4- yl)methyl)-2,5-diazabicyclo[2.2.2]octan-2-yl)- 2-(2,6-dioxopiperidin-3-yl)isoindoline-1,3- dione |
| | 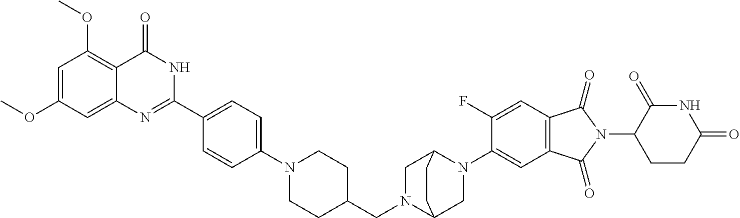 [SMILES: COc1cc(OC)c2c(=O)[nH]c(-c3ccc(N4CCC(CN5CC6CCC5CN6c5cc6c(cc5F)C(=O)N(C5CCC(=O)NC5=O)C6=O)CC4)cc3)nc2c1] | 5-(5-((1-(4-(5,7-dimethoxy-4-oxo-3,4- dihydroquinazolin-2-yl)phenyl)piperidin-4- yl)methyl)-2,5-diazabicyclo[2.2.2]octan-2-yl)- 2-(2,6-dioxopiperidin-3-yl)-6- fluoroisoindoline-1,3-dione |
| | 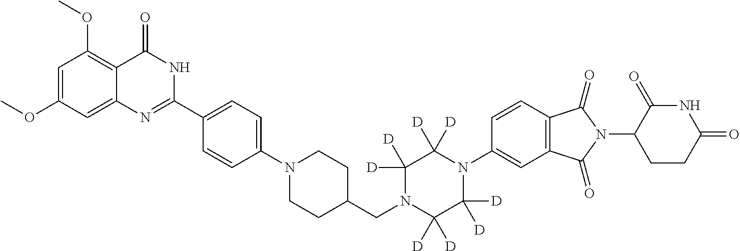 [SMILES: [2H]C1([2H])N(CC2CCN(c3ccc(-c4nc5cc(OC)cc(OC)c5c(=O)[nH]4)cc3)CC2)C([2H])([2H])C([2H])([2H])N(c2ccc3c(c2)C(=O)N(C2CCC(=O)NC2=O)C3=O)C1([2H])[2H]] | 5-(4-((1-(4-(5,7-dimethoxy-4-oxo-3,4- dihydroquinazolin-2-yl)phenyl)piperidin-4- yl)methyl)piperazin-1-yl-2,2,3,3,5,5,6,6-d₈)-2- (2,6-dioxopiperidin-3-yl)isoindoline-1,3-dione |
| | 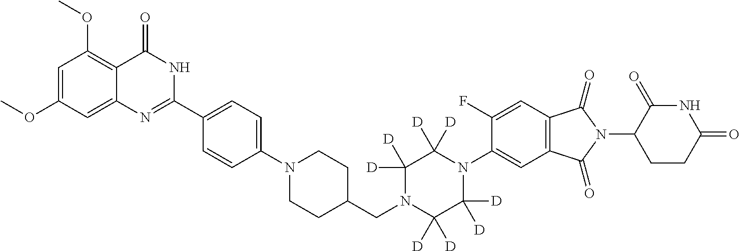 [SMILES: [2H]C1([2H])N(CC2CCN(c3ccc(-c4nc5cc(OC)cc(OC)c5c(=O)[nH]4)cc3)CC2)C([2H])([2H])C([2H])([2H])N(c2cc3c(cc2F)C(=O)N(C2CCC(=O)NC2=O)C3=O)C1([2H])[2H]] | 5-(4-((1-(4-(5,7-dimethoxy-4-oxo-3,4- dihydroquinazolin-2-yl)phenyl)piperidin-4- yl)methyl)piperazin-1-yl-2,2,3,3,5,5,6,6-d₈)-2- (2,6-dioxopiperidin-3-yl)-6-fluoroisoindoline- 1,3-dione |
| | 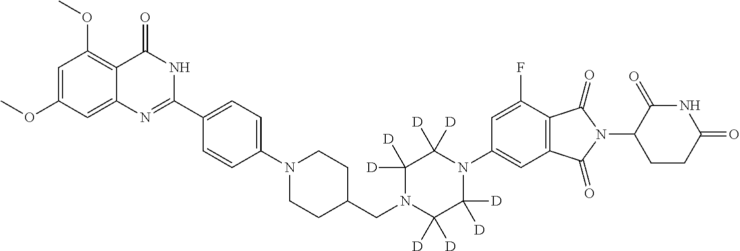 [SMILES: [2H]C1([2H])N(CC2CCN(c3ccc(-c4nc5cc(OC)cc(OC)c5c(=O)[nH]4)cc3)CC2)C([2H])([2H])C([2H])([2H])N(c2cc(F)c3c(c2)C(=O)N(C2CCC(=O)NC2=O)C3=O)C1([2H])[2H]] | 6-(4-((1-(4-(5,7-dimethoxy-4-oxo-3,4- dihydroquinazolin-2-yl)phenyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 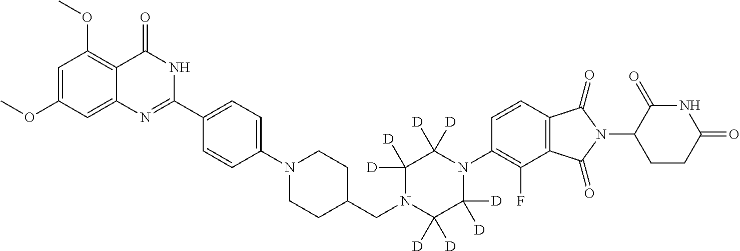 [SMILES: [2H]C1([2H])N(CC2CCN(c3ccc(-c4nc5cc(OC)cc(OC)c5c(=O)[nH]4)cc3)CC2)C([2H])([2H])C([2H])([2H])N(c2ccc3c(c2F)C(=O)N(C2CCC(=O)NC2=O)C3=O)C1([2H])[2H]] | 5-(4-((1-(4-(5,7-dimethoxy-4-oxo-3,4- dihydroquinazolin-2-yl)phenyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 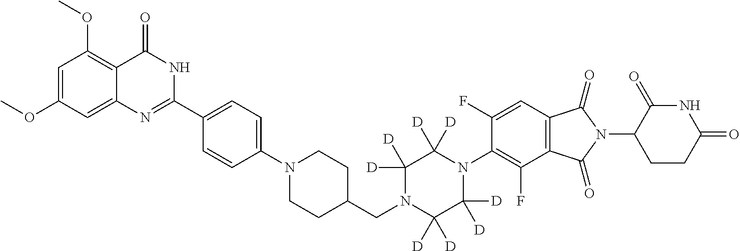 [SMILES: [2H]C1([2H])N(CC2CCN(c3ccc(-c4nc5cc(OC)cc(OC)c5c(=O)[nH]4)cc3)CC2)C([2H])([2H])C([2H])([2H])N(c2c(F)cc3c(c2F)C(=O)N(C2CCC(=O)NC2=O)C3=O)C1([2H])[2H]] | 5-(4-((1-(4-(5,7-dimethoxy-4-oxo-3,4- dihydroquinazolin-2-yl)phenyl)piperidin-4- yl)methyl)piperazin-1-yl-2,2,3,3,5,5,6,6-d₈)-2- (2,6-dioxopiperidin-3-yl)-4,6- difluoroisoindoline-1,3-dione |
| | 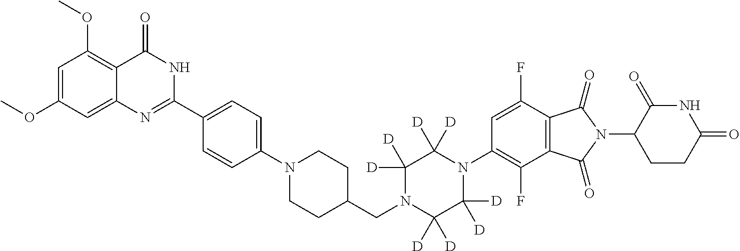 [SMILES: [2H]C1([2H])N(CC2CCN(c3ccc(-c4nc5cc(OC)cc(OC)c5c(=O)[nH]4)cc3)CC2)C([2H])([2H])C([2H])([2H])N(c2cc(F)c3c(c2F)C(=O)N(C2CCC(=O)NC2=O)C3=O)C1([2H])[2H]] | 5-(4-((1-(4-(5,7-dimethoxy-4-oxo-3,4- dihydroquinazolin-2-yl)phenyl)piperidin-4- yl)methyl)piperazin-1-yl-2,2,3,3,5,5,6,6-d₈)-2- (2,6-dioxopiperidin-3-yl)-4,7- difluoroisoindoline-1,3-dione |
| | 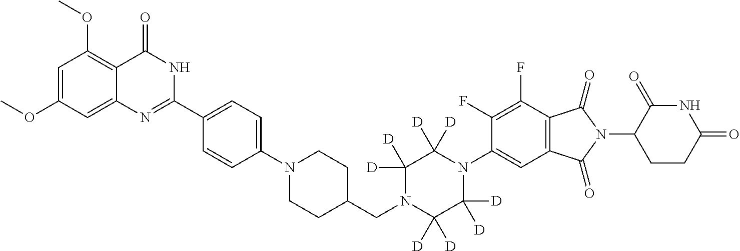 [SMILES: [2H]C1([2H])N(CC2CCN(c3ccc(-c4nc5cc(OC)cc(OC)c5c(=O)[nH]4)cc3)CC2)C([2H])([2H])C([2H])([2H])N(c2cc3c(c(F)c2F)C(=O)N(C2CCC(=O)NC2=O)C3=O)C1([2H])[2H]] | 6-(4-((1-(4-(5,7-dimethoxy-4-oxo-3,4- dihydroquinazolin-2-yl)phenyl)piperidin-4- yl)methyl)piperazin-1-yl-2,2,3,3,5,5,6,6-d₈)-2- (2,6-dioxopiperidin-3-yl)-4,5- difluoroisoindoline-1,3-dione |
| | 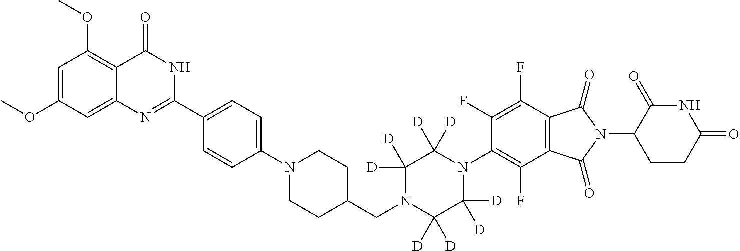 [SMILES: [2H]C1([2H])N(CC2CCN(c3ccc(-c4nc5cc(OC)cc(OC)c5c(=O)[nH]4)cc3)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(4-(5,7-dimethoxy-4-oxo-3,4- dihydroquinazolin-2-yl)phenyl)piperidin-4- yl)methyl)piperazin-1-yl-2,2,3,3,5,5,6,6-d₈)-2- (2,6-dioxopiperidin-3-yl)-4,6,7- trifluoroisoindoline-1,3-dione |
| | 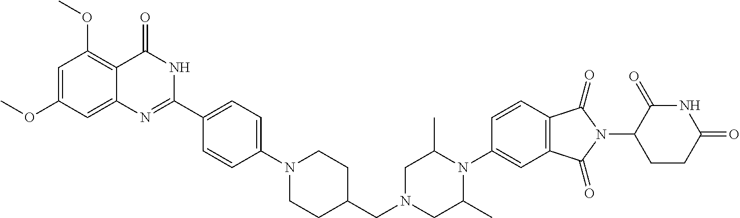 [SMILES: COc1cc(OC)c2c(=O)[nH]c(-c3ccc(N4CCC(CN5CC(C)N(c6ccc7c(c6)C(=O)N(C6CCC(=O)NC6=O)C7=O)C(C)C5)CC4)cc3)nc2c1] | 5-(4-((1-(4-(5,7-dimethoxy-4-oxo-3,4- dihydroquinazolin-2-yl)phenyl)piperidin-4- yl)methyl)-2,6-dimethylpiperazin-1-yl)-2-(2,6- dioxopiperidin-3-yl)isoindoline-1,3-dione |
| | 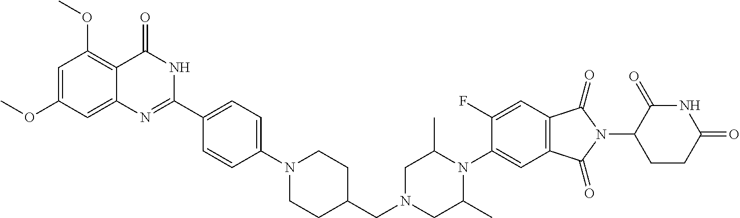 [SMILES: COc1cc(OC)c2c(=O)[nH]c(-c3ccc(N4CCC(CN5CC(C)N(c6cc7c(cc6F)C(=O)N(C6CCC(=O)NC6=O)C7=O)C(C)C5)CC4)cc3)nc2c1] | 5-(4-((1-(4-(5,7-dimethoxy-4-oxo-3,4- dihydroquinazolin-2-yl)phenyl)piperidin-4- yl)methyl)-2,6-dimethylpiperazin-1-yl)-2-(2,6- dioxopiperidin-3-yl)-6-fluoroisoindoline-1,3- dione |
| | 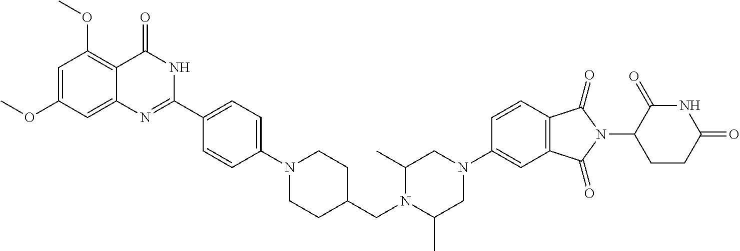 [SMILES: COc1cc(OC)c2c(=O)[nH]c(-c3ccc(N4CCC(CN5C(C)CN(c6ccc7c(c6)C(=O)N(C6CCC(=O)NC6=O)C7=O)CC5C)CC4)cc3)nc2c1] | 5-(4-((1-(4-(5,7-dimethoxy-4-oxo-3,4- dihydroquinazolin-2-yl)phenyl)piperidin-4- yl)methyl)-3,5-dimethylpiperazin-1-yl)-2-(2,6- dioxopiperidin-3-yl)isoindoline-1,3-dione |
| | 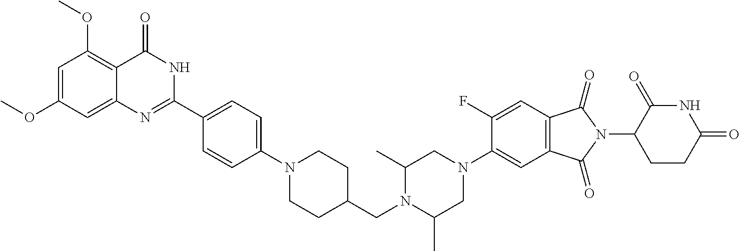 [SMILES: COc1cc(OC)c2c(=O)[nH]c(-c3ccc(N4CCC(CN5C(C)CN(c6cc7c(cc6F)C(=O)N(C6CCC(=O)NC6=O)C7=O)CC5C)CC4)cc3)nc2c1] | 5-(4-((1-(4-(5,7-dimethoxy-4-oxo-3,4- dihydroquinazolin-2-yl)phenyl)piperidin-4- yl)methyl)-3,5-dimethylpiperazin-1-yl)-2-(2,6- dioxopiperidin-3-yl)-6-fluoroisoindoline-1,3- dione |
| | 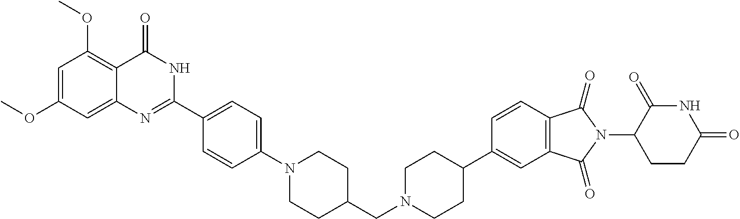 [SMILES: COc1cc(OC)c2c(=O)[nH]c(-c3ccc(N4CCC(CN5CCC(c6ccc7c(c6)C(=O)N(C6CCC(=O)NC6=O)C7=O)CC5)CC4)cc3)nc2c1] | 5-(1-((1-(4-(5,7-dimethoxy-4-oxo-3,4- dihydroquinazolin-2-yl)phenyl)piperidin-4- yl)methyl)piperidin-4-yl)-2-(2,6- dioxopiperidin-3-yl)isoindoline-1,3-dione |
| | 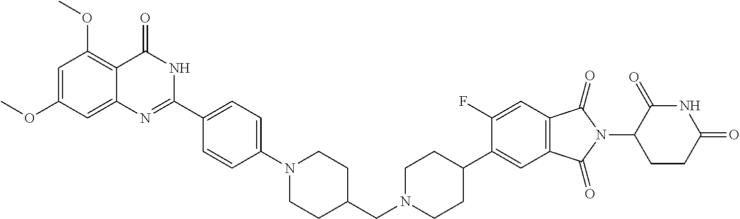 [SMILES: COc1cc(OC)c2c(=O)[nH]c(-c3ccc(N4CCC(CN5CCC(c6cc7c(cc6F)C(=O)N(C6CCC(=O)NC6=O)C7=O)CC5)CC4)cc3)nc2c1] | 5-(1-((1-(4-(5,7-dimethoxy-4-oxo-3,4- dihydroquinazolin-2-yl)phenyl)piperidin-4- yl)methyl)piperidin-4-yl)-2-(2,6- dioxopiperidin-3-yl)-6-fluoroisoindoline-1,3- dione |
| | 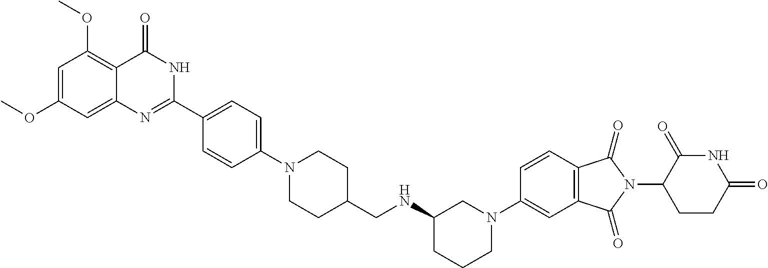 [SMILES: COc1cc(OC)c2c(=O)[nH]c(-c3ccc(N4CCC(CN[C@@H]5CCCN(c6ccc7c(c6)C(=O)N(C6CCC(=O)NC6=O)C7=O)C5)CC4)cc3)nc2c1] | 5-((R)-3-(((1-(4-(5,7-dimethoxy-4-oxo-3,4- dihydroquinazolin-2-yl)phenyl)piperidin-4- yl)methyl)amino)piperidin-1-yl)-2-(2,6- dioxopiperidin-3-yl)isoindoline-1,3-dione |
| | 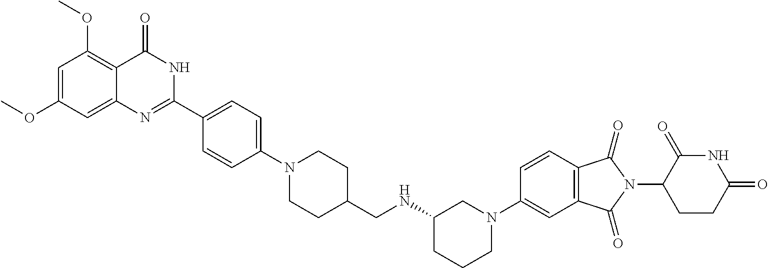 [SMILES: COc1cc(OC)c2c(=O)[nH]c(-c3ccc(N4CCC(CN[C@H]5CCCN(c6ccc7c(c6)C(=O)N(C6CCC(=O)NC6=O)C7=O)C5)CC4)cc3)nc2c1] | 5-((S)-3-(((1-(4-(5,7-dimethoxy-4-oxo-3,4- dihydroquinazolin-2-yl)phenyl)piperidin-4- yl)methyl)amino)piperidin-1-yl)-2-(2,6- dioxopiperidin-3-yl)isoindoline-1,3-dione |
| | 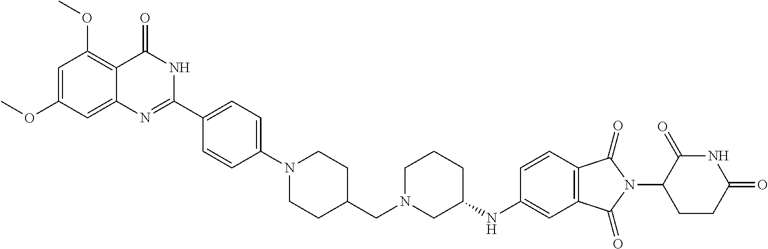 [SMILES: COc1cc(OC)c2c(=O)[nH]c(-c3ccc(N4CCC(CN5CCC[C@H](Nc6ccc7c(c6)C(=O)N(C6CCC(=O)NC6=O)C7=O)C5)CC4)cc3)nc2c1] | 5-(((S)-1-((1-(4-(5,7-dimethoxy-4-oxo-3,4- dihydroquinazolin-2-yl)phenyl)piperidin-4- yl)methyl)piperidin-3-yl)amino)-2-(2,6- dioxopiperidin-3-yl)isoindoline-1,3-dione |
| | 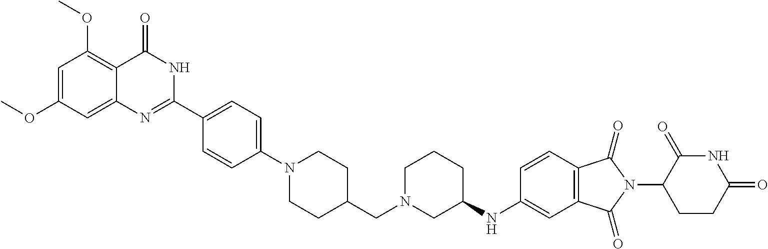 [SMILES: COc1cc(OC)c2c(=O)[nH]c(-c3ccc(N4CCC(CN5CCC[C@@H](Nc6ccc7c(c6)C(=O)N(C6CCC(=O)NC6=O)C7=O)C5)CC4)cc3)nc2c1] | 5-(((R)-1-((1-(4-(5,7-dimethoxy-4-oxo-3,4- dihydroquinazolin-2-yl)phenyl)piperidin-4- yl)methyl)piperidin-3-yl)amino)-2-(2,6- dioxopiperidin-3-yl)isoindoline-1,3-dione |
| | 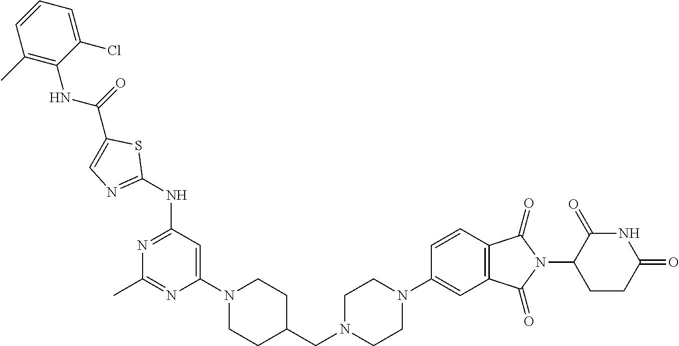 [SMILES: Cc1nc(Nc2ncc(C(=O)Nc3c(C)cccc3Cl)s2)cc(N2CCC(CN3CCN(c4ccc5c(c4)C(=O)N(C4CCC(=O)NC4=O)C5=O)CC3)CC2)n1] | N-(2-chloro-6-methylphenyl)-2-((6-(4-((4-(2- (2,6-dioxopiperidin-3-yl)-1,3-dioxoisoindolin- 5-yl)piperazin-1-yl)methyl)piperidin-1-yl)-2- methylpyrimidin-4-yl)amino)thiazole-5- carboxamide |
| | 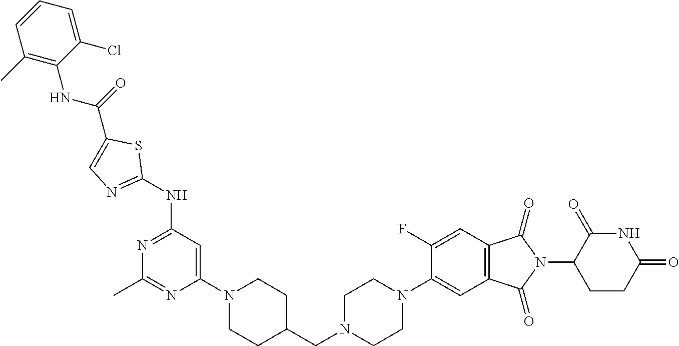 [SMILES: Cc1nc(Nc2ncc(C(=O)Nc3c(C)cccc3Cl)s2)cc(N2CCC(CN3CCN(c4cc5c(cc4F)C(=O)N(C4CCC(=O)NC4=O)C5=O)CC3)CC2)n1] | N-(2-chloro-6-methylphenyl)-2-((6-(4-((4-(2- (2,6-dioxopiperidin-3-yl)-6-fluoro-1,3- dioxoisoindolin-5-yl)piperazin-1- yl)methyl)piperidin-1-yl)-2-methylpyrimidin-4- yl)amino)thiazole-5-carboxamide |
| GT-03630 | 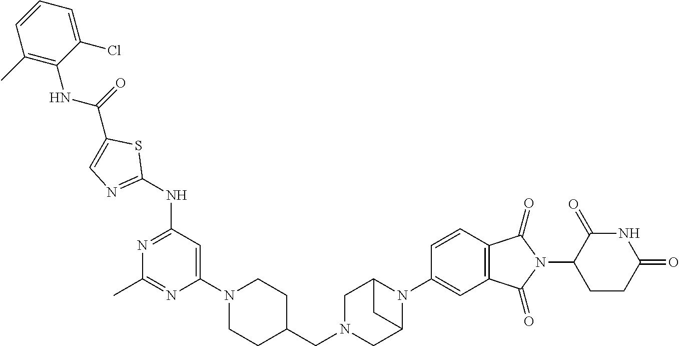 [SMILES: Cc1nc(Nc2ncc(C(=O)Nc3c(C)cccc3Cl)s2)cc(N2CCC(CN3CC4CC(C3)N4c3ccc4c(c3)C(=O)N(C3CCC(=O)NC3=O)C4=O)CC2)n1] | N-(2-chloro-6-methylphenyl)-2-((6-(4-((6-(2- (2,6-dioxopiperidin-3-yl)-1,3-dioxoisoindolin- 5-yl)-3,6-diazabicyclo[3.1.1]heptan-3- yl)methyl)piperidin-1-yl)-2-methylpyrimidin-4- yl)amino)thiazole-5-carboxamide |
| GT-03628 | 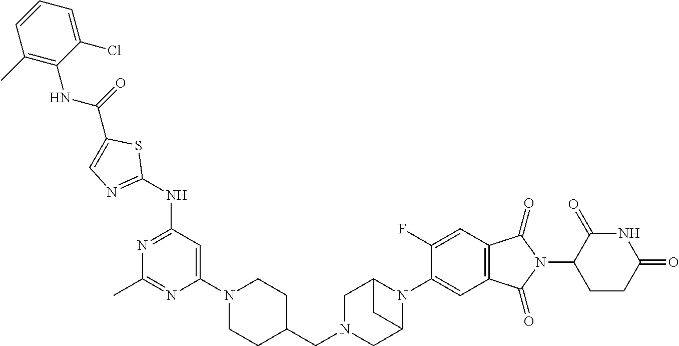 [SMILES: Cc1nc(Nc2ncc(C(=O)Nc3c(C)cccc3Cl)s2)cc(N2CCC(CN3CC4CC(C3)N4c3cc4c(cc3F)C(=O)N(C3CCC(=O)NC3=O)C4=O)CC2)n1] | N-(2-chloro-6-methylphenyl)-2-((6-(4-((6-(2- (2,6-dioxopiperidin-3-yl)-6-fluoro-1,3- dioxoisoindolin-5-yl)-3,6- diazabicyclo[3.1.1]heptan-3- yl)methyl)piperidin-1-yl)-2-methylpyrimidin-4- yl)amino)thiazole-5-carboxamide |
| GT-03631 | 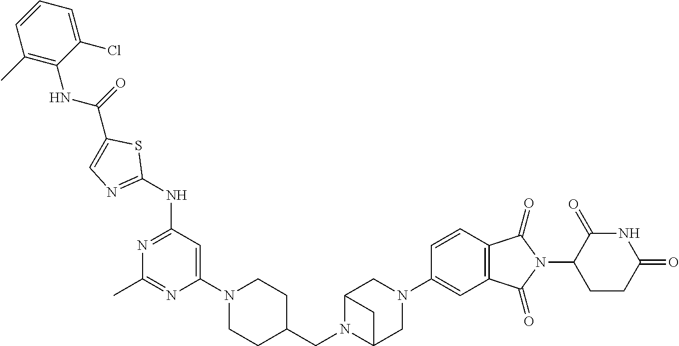 [SMILES: Cc1nc(Nc2ncc(C(=O)Nc3c(C)cccc3Cl)s2)cc(N2CCC(CN3C4CC3CN(c3ccc5c(c3)C(=O)N(C3CCC(=O)NC3=O)C5=O)C4)CC2)n1] | N-(2-chloro-6-methylphenyl)-2-((6-(4-((3-(2- (2,6-dioxopiperidin-3-yl)-1,3-dioxoisoindolin- 5-yl)-3,6-diazabicyclo[3.1.1]heptan-6- yl)methyl)piperidin-1-yl)-2-methylpyrimidin-4- yl)amino)thiazole-5-carboxamide |
| GT-03538 | 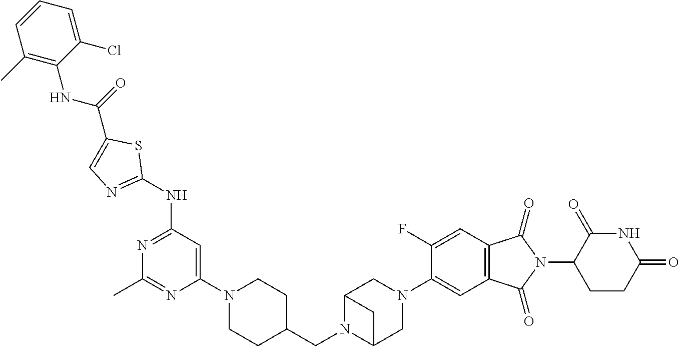 [SMILES: Cc1nc(Nc2ncc(C(=O)Nc3c(C)cccc3Cl)s2)cc(N2CCC(CN3C4CC3CN(c3cc5c(cc3F)C(=O)N(C3CCC(=O)NC3=O)C5=O)C4)CC2)n1] | N-(2-chloro-6-methylphenyl)-2-((6-(4-((3-(2- (2,6-dioxopiperidin-3-yl)-6-fluoro-1,3- dioxoisoindolin-5-yl)-3,6- diazabicyclo[3.1.1]heptan-6- yl)methyl)piperidin-1-yl)-2-methylpyrimidin-4- yl)amino)thiazole-5-carboxamide |
| GT-03629 | 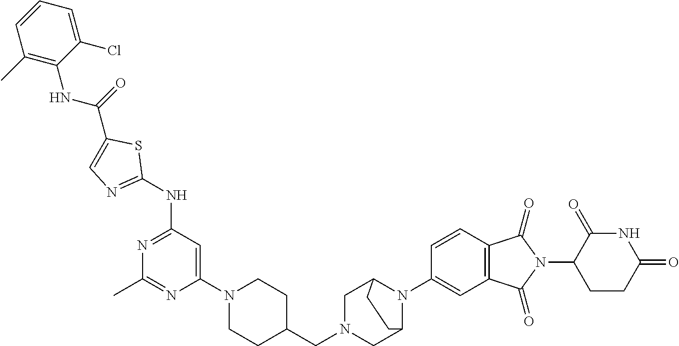 [SMILES: Cc1nc(Nc2ncc(C(=O)Nc3c(C)cccc3Cl)s2)cc(N2CCC(CN3CC4CCC(C3)N4c3ccc4c(c3)C(=O)N(C3CCC(=O)NC3=O)C4=O)CC2)n1] | N-(2-chloro-6-methylphenyl)-2-((6-(4-((8-(2- (2,6-dioxopiperidin-3-yl)-1,3-dioxoisoindolin- 5-yl)-3,8-diazabicyclo[3.2.1]octan-3- yl)methyl)piperidin-1-yl)-2-methylpyrimidin-4- yl)amino)thiazole-5-carboxamide |
| GT-03627 | 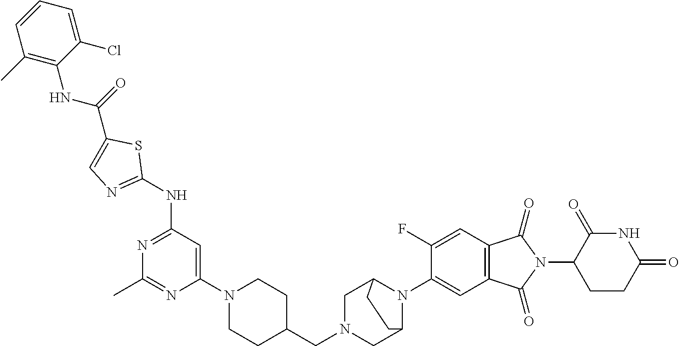 [SMILES: Cc1nc(Nc2ncc(C(=O)Nc3c(C)cccc3Cl)s2)cc(N2CCC(CN3CC4CCC(C3)N4c3cc4c(cc3F)C(=O)N(C3CCC(=O)NC3=O)C4=O)CC2)n1] | N-(2-chloro-6-methylphenyl)-2-((6-(4-((8-(2- (2,6-dioxopiperidin-3-yl)-6-fluoro-1,3- dioxoisoindolin-5-yl)-3,8- diazabicyclo[3.2.1]octan-3-yl)methyl)piperidin- 1-yl)-2-methylpyrimidin-4-yl)amino)thiazole- 5-carboxamide |
| GT-03632 | 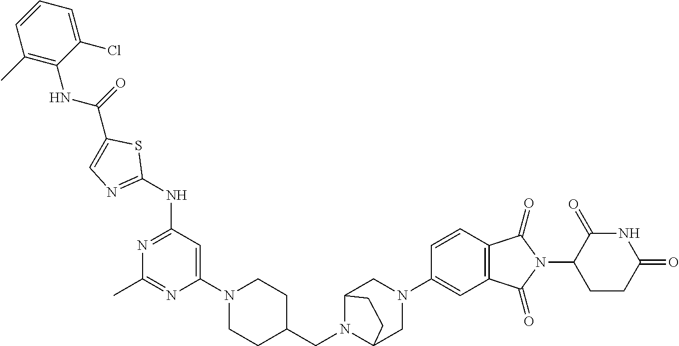 [SMILES: Cc1nc(Nc2ncc(C(=O)Nc3c(C)cccc3Cl)s2)cc(N2CCC(CN3C4CCC3CN(c3ccc5c(c3)C(=O)N(C3CCC(=O)NC3=O)C5=O)C4)CC2)n1] | N-(2-chloro-6-methylphenyl)-2-((6-(4-((3-(2- (2,6-dioxopiperidin-3-yl)-1,3-dioxoisoindolin- 5-yl)-3,8-diazabicyclo[3.2.1]octan-8- yl)methyl)piperidin-1-yl)-2-methylpyrimidin-4- yl)amino)thiazole-5-carboxamide |
| GT-03537 | 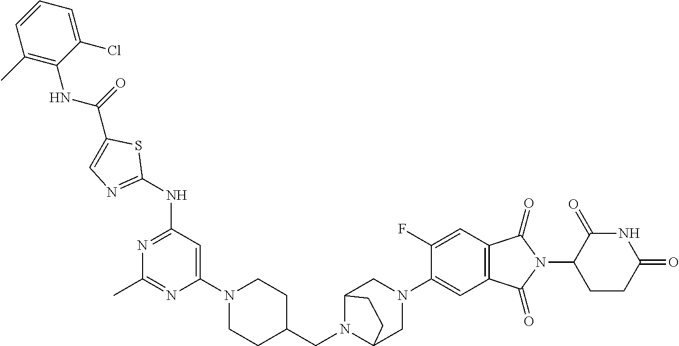 [SMILES: Cc1nc(Nc2ncc(C(=O)Nc3c(C)cccc3Cl)s2)cc(N2CCC(CN3C4CCC3CN(c3cc5c(cc3F)C(=O)N(C3CCC(=O)NC3=O)C5=O)C4)CC2)n1] | N-(2-chloro-6-methylphenyl)-2-((6-(4-((3-(2- (2,6-dioxopiperidin-3-yl)-6-fluoro-1,3- dioxoisoindolin-5-yl)-3,8- diazabicyclo[3.2.1]octan-8-yl)methyl)piperidin- 1-yl)-2-methylpyrimidin-4-yl)amino)thiazole- 5-carboxamide |
| | 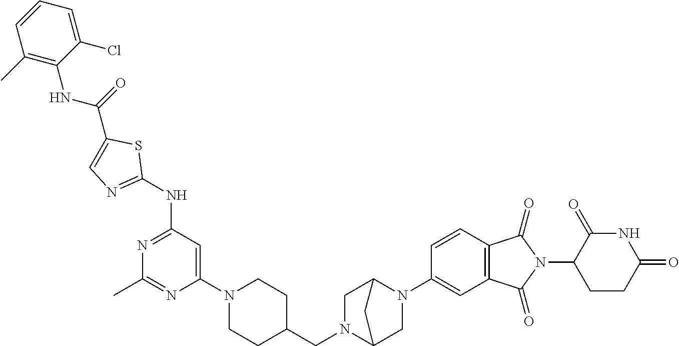 [SMILES: Cc1nc(Nc2ncc(C(=O)Nc3c(C)cccc3Cl)s2)cc(N2CCC(CN3CC4CC3CN4c3ccc4c(c3)C(=O)N(C3CCC(=O)NC3=O)C4=O)CC2)n1] | N-(2-chloro-6-methylphenyl)-2-((6-(4-((5-(2- (2,6-dioxopiperidin-3-yl)-1,3-dioxoisoindolin- 5-yl)-2,5-diazabicyclo[2.2.1]heptan-2- yl)methyl)piperidin-1-yl)-2-methylpyrimidin-4- yl)amino)thiazole-5-carboxamide |
| | 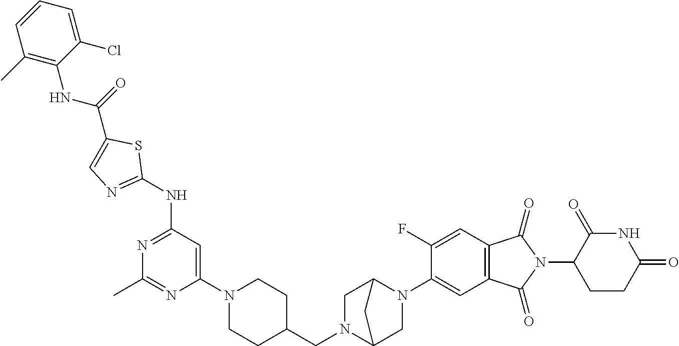 [SMILES: Cc1nc(Nc2ncc(C(=O)Nc3c(C)cccc3Cl)s2)cc(N2CCC(CN3CC4CC3CN4c3cc4c(cc3F)C(=O)N(C3CCC(=O)NC3=O)C4=O)CC2)n1] | N-(2-chloro-6-methylphenyl)-2-((6-(4-((5-(2- (2,6-dioxopiperidin-3-yl)-6-fluoro-1,3- dioxoisoindolin-5-yl)-2,5- diazabicyclo[2.2.1]heptan-2- yl)methyl)piperidin-1-yl)-2-methylpyrimidin-4- yl)amino)thiazole-5-carboxamide |
| | 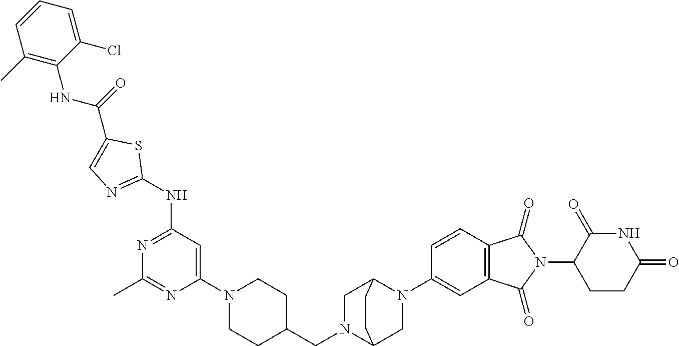 [SMILES: Cc1nc(Nc2ncc(C(=O)Nc3c(C)cccc3Cl)s2)cc(N2CCC(CN3CC4CCC3CN4c3ccc4c(c3)C(=O)N(C3CCC(=O)NC3=O)C4=O)CC2)n1] | N-(2-chloro-6-methylphenyl)-2-((6-(4-((5-(2- (2,6-dioxopiperidin-3-yl)-1,3-dioxoisoindolin- 5-yl)-2,5-diazabicyclo[2.2.2]octan-2- yl)methyl)piperidin-1-yl)-2-methylpyrimidin-4- yl)amino)thiazole-5-carboxamide |
| | 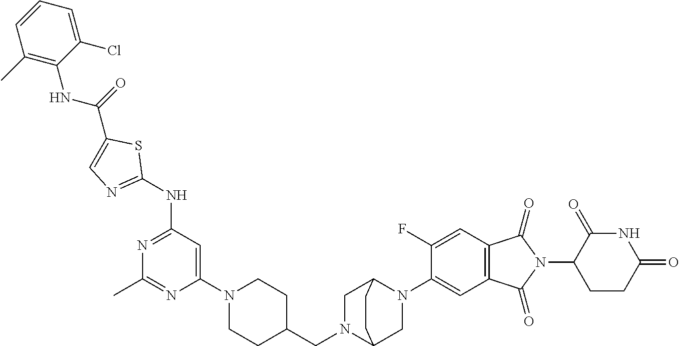 [SMILES: Cc1nc(Nc2ncc(C(=O)Nc3c(C)cccc3Cl)s2)cc(N2CCC(CN3CC4CCC3CN4c3cc4c(cc3F)C(=O)N(C3CCC(=O)NC3=O)C4=O)CC2)n1] | N-(2-chloro-6-methylphenyl)-2-((6-(4-((5-(2- (2,6-dioxopiperidin-3-yl)-6-fluoro-1,3- dioxoisoindolin-5-yl)-2,5- diazabicyclo[2.2.2]octan-2-yl)methyl)piperidin- 1-yl)-2-methylpyrimidin-4-yl)amino)thiazole- 5-carboxamide |
| | 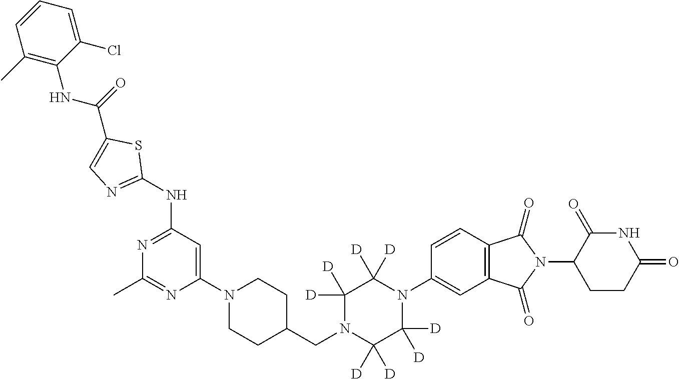 [SMILES: [2H]C1([2H])N(CC2CCN(c3cc(Nc4ncc(C(=O)Nc5c(C)cccc5Cl)s4)nc(C)n3)CC2)C([2H])([2H])C([2H])([2H])N(c2ccc3c(c2)C(=O)N(C2CCC(=O)NC2=O)C3=O)C1([2H])[2H]] | N-(2-chloro-6-methylphenyl)-2-((6-(4-((4-(2- (2,6-dioxopiperidin-3-yl)-1,3-dioxoisoindolin- 5-yl)piperazin-1-yl-2,2,3,3,5,5,6,6- d₈)methyl)piperidin-1-yl)-2-methylpyrimidin-4- yl)amino)thiazole-5-carboxamide |
| | 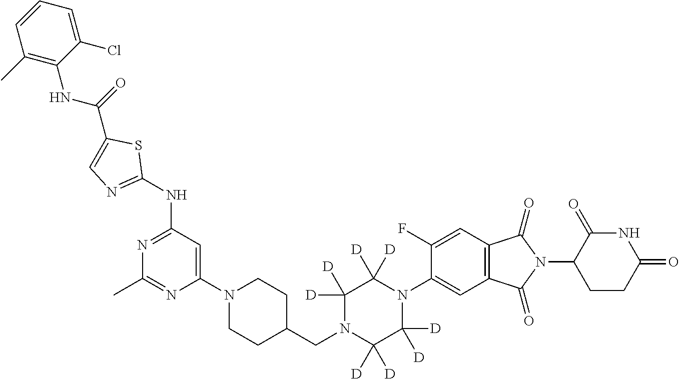 [SMILES: [2H]C1([2H])N(CC2CCN(c3cc(Nc4ncc(C(=O)Nc5c(C)cccc5Cl)s4)nc(C)n3)CC2)C([2H])([2H])C([2H])([2H])N(c2cc3c(cc2F)C(=O)N(C2CCC(=O)NC2=O)C3=O)C1([2H])[2H]] | N-(2-chloro-6-methylphenyl)-2-((6-(4-((4-(2- (2,6-dioxopiperidin-3-yl)-6-fluoro-1,3- dioxoisoindolin-5-yl)piperazin-1-yl- 2,2,3,3,5,5,6,6-d₈)methyl)piperidin-1-yl)-2- methylpyrimidin-4-yl)amino)thiazole-5- carboxamide |
| | 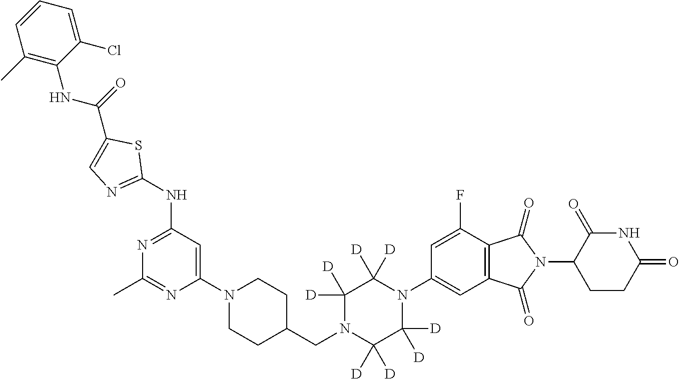 [SMILES: [2H]C1([2H])N(CC2CCN(c3cc(Nc4ncc(C(=O)Nc5c(C)cccc5Cl)s4)nc(C)n3)CC2)C([2H])([2H])C([2H])([2H])N(c2cc(F)c3c(c2)C(=O)N(C2CCC(=O)NC2=O)C3=O)C1([2H])[2H]] | N-(2-chloro-6-methylphenyl)-2-((6-(4-((4-(2- (2,6-dioxopiperidin-3-yl)-7-fluoro-1,3- dioxoisoindolin-5-yl)piperazin-1-yl- 2,2,3,3,5,5,6,6-d₈)methyl)piperidin-1-yl)-2- methylpyrimidin-4-yl)amino)thiazole-5- carboxamide |
| | 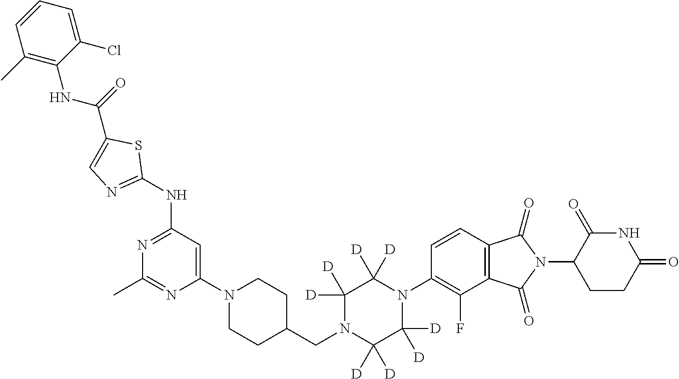 [SMILES: [2H]C1([2H])N(CC2CCN(c3cc(Nc4ncc(C(=O)Nc5c(C)cccc5Cl)s4)nc(C)n3)CC2)C([2H])([2H])C([2H])([2H])N(c2ccc3c(c2F)C(=O)N(C2CCC(=O)NC2=O)C3=O)C1([2H])[2H]] | N-(2-chloro-6-methylphenyl)-2-((6-(4-((4-(2- (2,6-dioxopiperidin-3-yl)-4-fluoro-1,3- dioxoisoindolin-5-yl)piperazin-1-yl- 2,2,3,3,5,5,6,6-d₈)methyl)piperidin-1-yl)-2- methylpyrimidin-4-yl)amino)thiazole-5- carboxamide |
| | 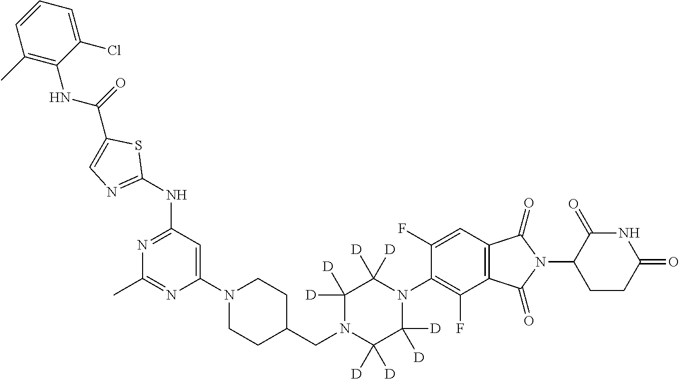 [SMILES: [2H]C1([2H])N(CC2CCN(c3cc(Nc4ncc(C(=O)Nc5c(C)cccc5Cl)s4)nc(C)n3)CC2)C([2H])([2H])C([2H])([2H])N(c2c(F)cc3c(c2F)C(=O)N(C2CCC(=O)NC2=O)C3=O)C1([2H])[2H]] | N-(2-chloro-6-methylphenyl)-2-((6-(4-((4-(2- (2,6-dioxopiperidin-3-yl)-4,6-difluoro-1,3- dioxoisoindolin-5-yl)piperazin-1-yl- 2,2,3,3,5,5,6,6-d₈)methyl)piperidin-1-yl)-2- methylpyrimidin-4-yl)amino)thiazole-5- carboxamide |
| | 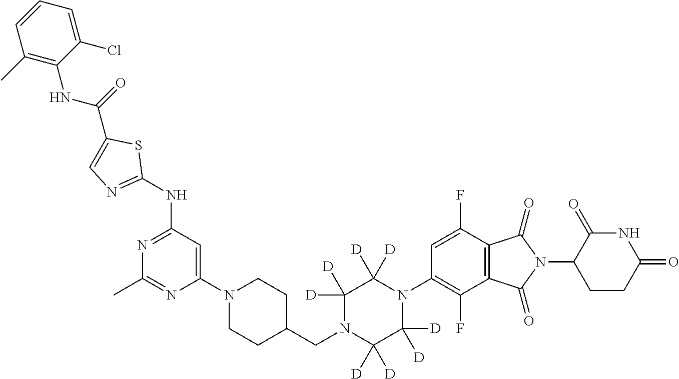 [SMILES: [2H]C1([2H])N(CC2CCN(c3cc(Nc4ncc(C(=O)Nc5c(C)cccc5Cl)s4)nc(C)n3)CC2)C([2H])([2H])C([2H])([2H])N(c2cc(F)c3c(c2F)C(=O)N(C2CCC(=O)NC2=O)C3=O)C1([2H])[2H]] | N-(2-chloro-6-methylphenyl)-2-((6-(4-((4-(2- (2,6-dioxopiperidin-3-yl)-4,7-difluoro-1,3- dioxoisoindolin-5-yl)piperazin-1-yl- 2,2,3,3,5,5,6,6-d₈)methyl)piperidin-1-yl)-2- methylpyrimidin-4-yl)amino)thiazole-5- carboxamide |
| | 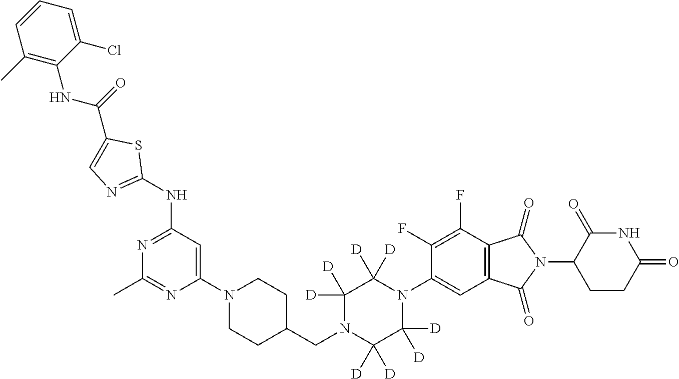 [SMILES: [2H]C1([2H])N(CC2CCN(c3cc(Nc4ncc(C(=O)Nc5c(C)cccc5Cl)s4)nc(C)n3)CC2)C([2H])([2H])C([2H])([2H])N(c2cc3c(c(F)c2F)C(=O)N(C2CCC(=O)NC2=O)C3=O)C1([2H])[2H]] | N-(2-chloro-6-methylphenyl)-2-((6-(4-((4-(2- (2,6-dioxopiperidin-3-yl)-6,7-difluoro-1,3- dioxoisoindolin-5-yl)piperazin-1-yl- 2,2,3,3,5,5,6,6-d₈)methyl)piperidin-1-yl)-2- methylpyrimidin-4-yl)amino)thiazole-5- carboxamide |
| | 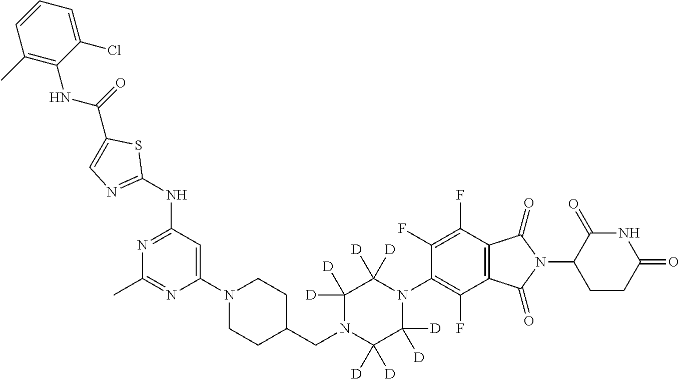 [SMILES: [2H]C1([2H])N(CC2CCN(c3cc(Nc4ncc(C(=O)Nc5c(C)cccc5Cl)s4)nc(C)n3)CC2)C([2H])([2H])C([2H])([2H])N(c2c(F)c(F)c3c(c2F)C(=O)N(C2CCC(=O)NC2=O)C3=O)C1([2H])[2H]] | N-(2-chloro-6-methylphenyl)-2-((6-(4-((4-(2- (2,6-dioxopiperidin-3-yl)-4,6,7-trifluoro-1,3- dioxoisoindolin-5-yl)piperazin-1-yl- 2,2,3,3,5,5,6,6-d₈)methyl)piperidin-1-yl)-2- methylpyrimidin-4-yl)amino)thiazole-5- carboxamide |
| | 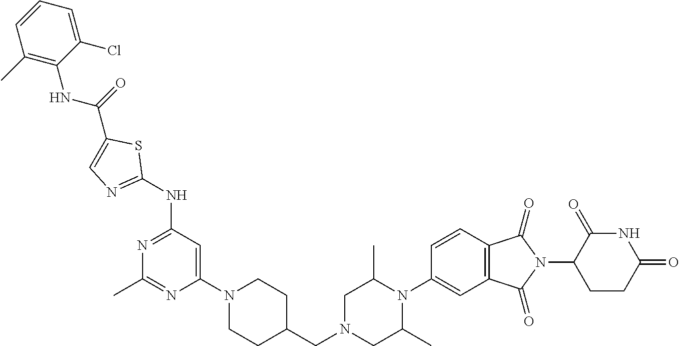 [SMILES: Cc1nc(Nc2ncc(C(=O)Nc3c(C)cccc3Cl)s2)cc(N2CCC(CN3CC(C)N(c4ccc5c(c4)C(=O)N(C4CCC(=O)NC4=O)C5=O)C(C)C3)CC2)n1] | N-(2-chloro-6-methylphenyl)-2-((6-(4-((4-(2- (2,6-dioxopiperidin-3-yl)-1,3-dioxoisoindolin- 5-yl)-3,5-dimethylpiperazin-1- yl)methyl)piperidin-1-yl)-2-methylpyrimidin-4- yl)amino)thiazole-5-carboxamide |
| | 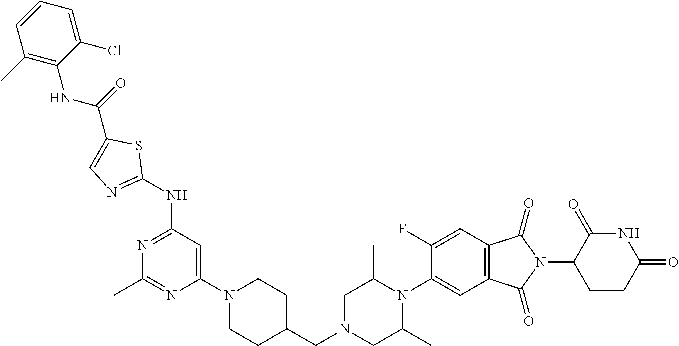 [SMILES: Cc1nc(Nc2ncc(C(=O)Nc3c(C)cccc3Cl)s2)cc(N2CCC(CN3CC(C)N(c4cc5c(cc4F)C(=O)N(C4CCC(=O)NC4=O)C5=O)C(C)C3)CC2)n1] | N-(2-chloro-6-methylphenyl)-2-((6-(4-((4-(2- (2,6-dioxopiperidin-3-yl)-6-fluoro-1,3- dioxoisoindolin-5-yl)-3,5-dimethylpiperazin-1 - yl)methyl)piperidin-1-yl)-2-methylpyrimidin-4- yl)amino)thiazole-5-carboxamide |
| | 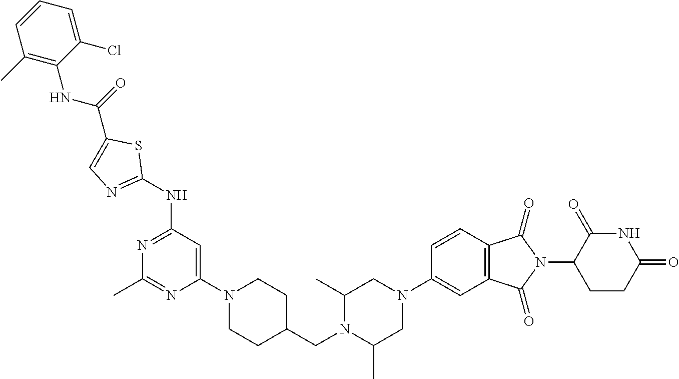 [SMILES: Cc1nc(Nc2ncc(C(=O)Nc3c(C)cccc3Cl)s2)cc(N2CCC(CN3C(C)CN(c4ccc5c(c4)C(=O)N(C4CCC(=O)NC4=O)C5=O)CC3C)CC2)n1] | N-(2-chloro-6-methylphenyl)-2-((6-(4-((4-(2- (2,6-dioxopiperidin-3-yl)-1,3-dioxoisoindolin- 5-yl)-2,6-dimethylpiperazin-1 - yl)methyl)piperidin-1-yl)-2-methylpyrimidin-4- yl)amino)thiazole-5-carboxamide |
| | 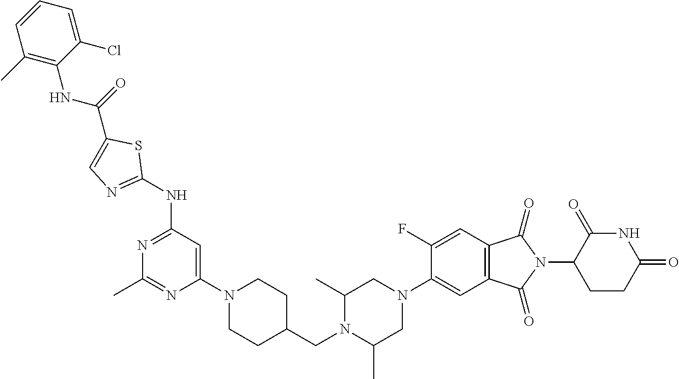 [SMILES: Cc1nc(Nc2ncc(C(=O)Nc3c(C)cccc3Cl)s2)cc(N2CCC(CN3C(C)CN(c4cc5c(cc4F)C(=O)N(C4CCC(=O)NC4=O)C5=O)CC3C)CC2)n1] | N-(2-chloro-6-methylphenyl)-2-((6-(4-((4-(2- (2,6-dioxopiperidin-3-yl)-6-fluoro-1,3- dioxoisoindolin-5-yl)-2,6-dimethylpiperazin-1- yl)methyl)piperidin-1-yl)-2-methylpyrimidin-4- yl)amino)thiazole-5-carboxamide |
| | 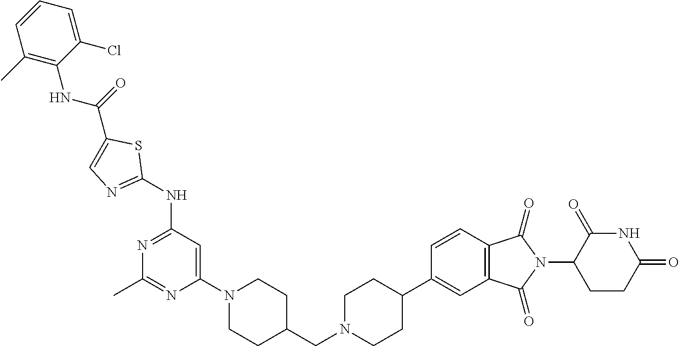 [SMILES: Cc1nc(Nc2ncc(C(=O)Nc3c(C)cccc3Cl)s2)cc(N2CCC(CN3CCC(c4ccc5c(c4)C(=O)N(C4CCC(=O)NC4=O)C5=O)CC3)CC2)n1] | N-(2-chloro-6-methylphenyl)-2-((6-(4-((4-(2- (2,6-dioxopiperidin-3-yl)-1,3-dioxoisoindolin- 5-yl)piperidin-1-yl)methyl)piperidin-1-yl)-2- methylpyrimidin-4-yl)amino)thiazole-5- carboxamide |
| | 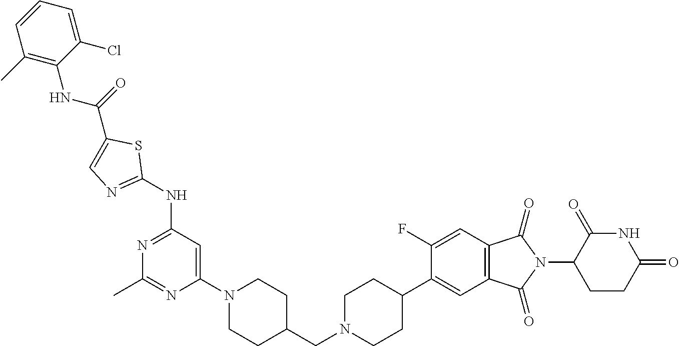 [SMILES: Cc1nc(Nc2ncc(C(=O)Nc3c(C)cccc3Cl)s2)cc(N2CCC(CN3CCC(c4cc5c(cc4F)C(=O)N(C4CCC(=O)NC4=O)C5=O)CC3)CC2)n1] | N-(2-chloro-6-methylphenyl)-2-((6-(4-((4-(2- (2,6-dioxopiperidin-3-yl)-6-fluoro-1,3- dioxoisoindolin-5-yl)piperidin-1- yl)methyl)piperidin-1-yl)-2-methylpyrimidin-4- yl)amino)thiazole-5-carboxamide |
| | 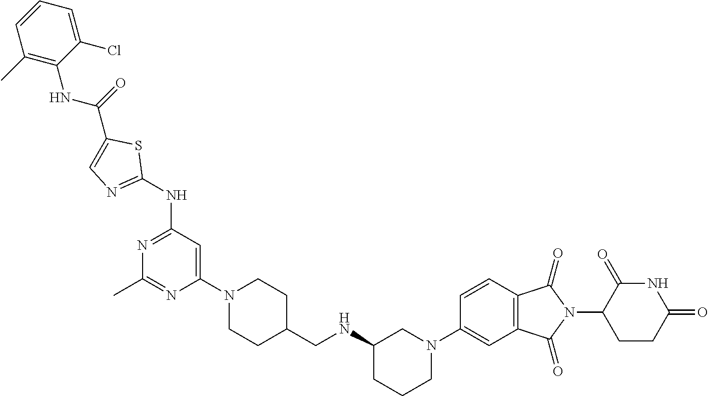 [SMILES: Cc1nc(Nc2ncc(C(=O)Nc3c(C)cccc3Cl)s2)cc(N2CCC(CN[C@@H]3CCCN(c4ccc5c(c4)C(=O)N(C4CCC(=O)NC4=O)C5=O)C3)CC2)n1] | N-(2-chloro-6-methylphenyl)-2-((6-(4-((((3R)- 1-(2-(2,6-dioxopiperidin-3-yl)-1,3- dioxoisoindolin-5-yl)piperidin-3- yl)amino)methyl)piperidin-1-yl)-2- methylpyrimidin-4-yl)amino)thiazole-5- carboxamide |
| | 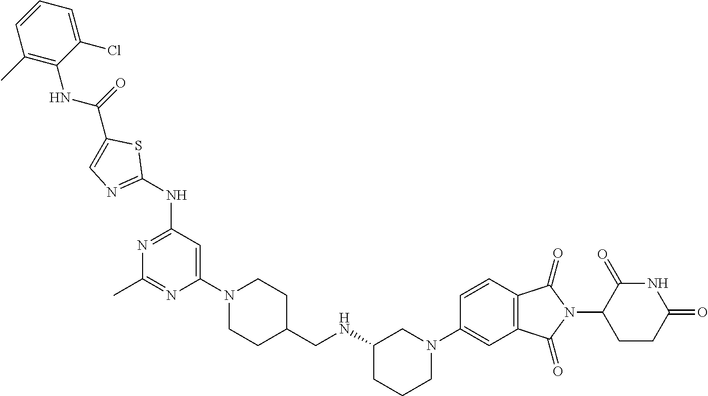 [SMILES: Cc1nc(Nc2ncc(C(=O)Nc3c(C)cccc3Cl)s2)cc(N2CCC(CN[C@H]3CCCN(c4ccc5c(c4)C(=O)N(C4CCC(=O)NC4=O)C5=O)C3)CC2)n1] | N-(2-chloro-6-methylphenyl)-2-((6-(4-((((3S)- 1-(2-(2,6-dioxopiperidin-3-yl)-1,3- dioxoisoindolin-5-yl)piperidin-3- yl)amino)methyl)piperidin-1-yl)-2- methylpyrimidin-4-yl)amino)thiazole-5- carboxamide |
| | 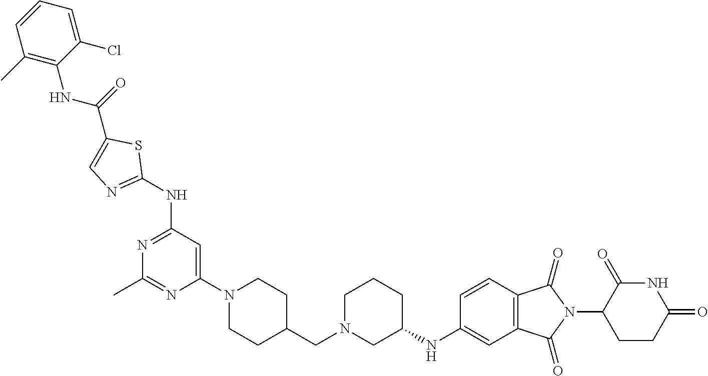 [SMILES: Cc1nc(Nc2ncc(C(=O)Nc3c(C)cccc3Cl)s2)cc(N2CCC(CN3CCC[C@H](Nc4ccc5c(c4)C(=O)N(C4CCC(=O)NC4=O)C5=O)C3)CC2)n1] | N-(2-chloro-6-methylphenyl)-2-((6-(4-(((3S)-3- ((2-(2,6-dioxopiperidin-3-yl)-1,3- dioxoisoindolin-5-yl)amino)piperidin-1- yl)methyl)piperidin-1-yl)-2-methylpyrimidin-4- yl)amino)thiazole-5-carboxamide |
| | 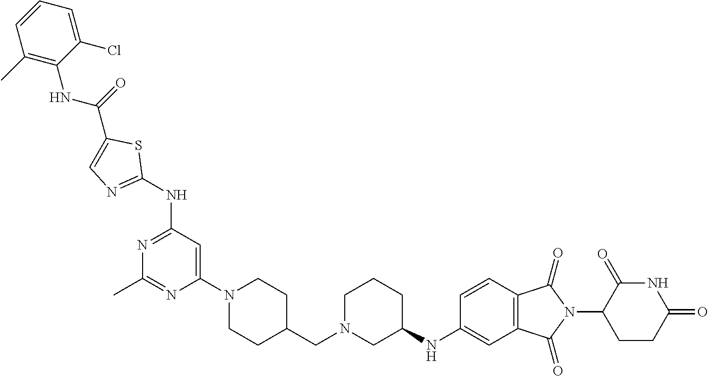 [SMILES: Cc1nc(Nc2ncc(C(=O)Nc3c(C)cccc3Cl)s2)cc(N2CCC(CN3CCC[C@@H](Nc4ccc5c(c4)C(=O)N(C4CCC(=O)NC4=O)C5=O)C3)CC2)n1] | N-(2-chloro-6-methylphenyl)-2-((6-(4-(((3R)- 3-((2-(2,6-dioxopiperidin-3-yl)-1,3- dioxoisoindolin-5-yl)amino)piperidin-1- yl)methyl)piperidin-1-yl)-2-methylpyrimidin-4- yl)amino)thiazole-5-carboxamide |
| | 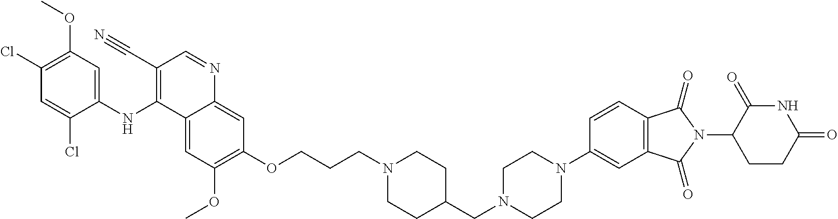 [SMILES: COc1cc(Nc2c(C#N)cnc3cc(OCCCN4CCC(CN5CCN(c6ccc7c(c6)C(=O)N(C6CCC(=O)NC6=O)C7=O)CC5)CC4)c(OC)cc23)c(Cl)cc1Cl] | 4-((2,4-dichloro-5-methoxyphenyl)amino)-7- (3-(4-((4-(2-(2,6-dioxopiperidin-3-yl)-1,3- dioxoisoindolin-5-yl)piperazin-1- yl)methyl)piperidin-1-yl)propoxy)-6- methoxyquinoline-3-carbonitrile |
| | 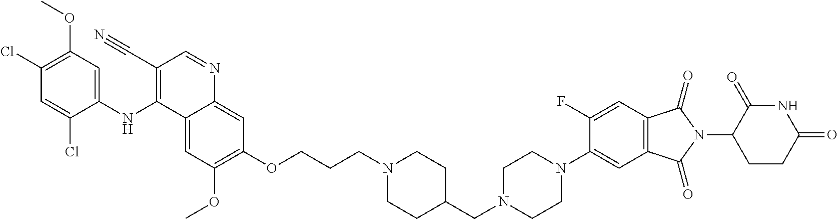 [SMILES: COc1cc(Nc2c(C#N)cnc3cc(OCCCN4CCC(CN5CCN(c6cc7c(cc6F)C(=O)N(C6CCC(=O)NC6=O)C7=O)CC5)CC4)c(OC)cc23)c(Cl)cc1Cl] | 4-((2,4-dichloro-5-methoxyphenyl)amino)-7- (3-(4-((4-(2-(2,6-dioxopiperidin-3-yl)-6-fluoro- 1,3-dioxoisoindolin-5-yl)piperazin-1- yl)methyl)piperidin-1-yl)propoxy)-6- methoxyquinoline-3-carbonitrile |
| | 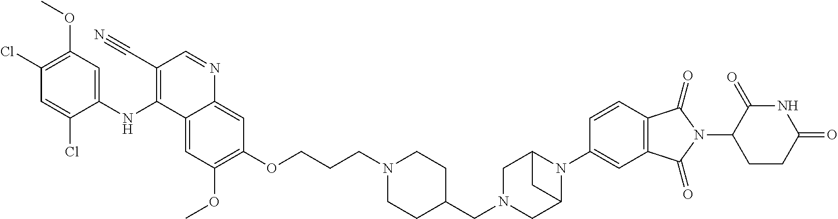 [SMILES: COc1cc(Nc2c(C#N)cnc3cc(OCCCN4CCC(CN5CC6CC(C5)N6c5ccc6c(c5)C(=O)N(C5CCC(=O)NC5=O)C6=O)CC4)c(OC)cc23)c(Cl)cc1Cl] | 4-((2,4-dichloro-5-methoxyphenyl)amino)-7- (3-(4-((6-(2-(2,6-dioxopiperidin-3-yl)-1,3- dioxoisoindolin-5-yl)-3,6- diazabicyclo[3.1.1]heptan-3- yl)methyl)piperidin-1-yl)propoxy)-6- methoxyquinoline-3-carbonitrile |
| | 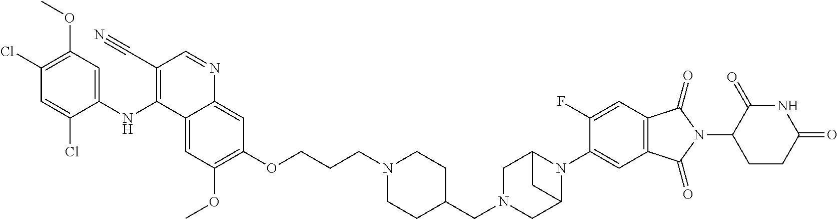 [SMILES: COc1cc(Nc2c(C#N)cnc3cc(OCCCN4CCC(CN5CC6CC(C5)N6c5cc6c(cc5F)C(=O)N(C5CCC(=O)NC5=O)C6=O)CC4)c(OC)cc23)c(Cl)cc1Cl] | 4-((2,4-dichloro-5-methoxyphenyl)amino)-7- (3-(4-((6-(2-(2,6-dioxopiperidin-3-yl)-6-fluoro- 1,3-dioxoisoindolin-5-yl)-3,6- diazabicyclo[3.1.1]heptan-3- yl)methyl)piperidin-1-yl)propoxy)-6- methoxyquinoline-3-carbonitrile |
| | 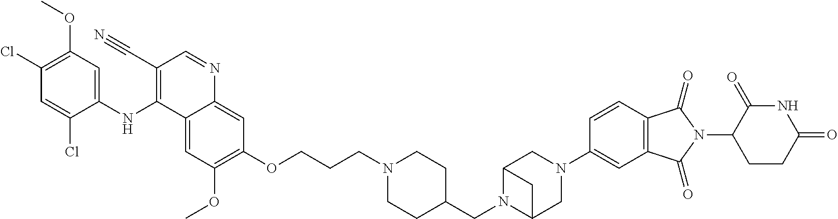 [SMILES: COc1cc(Nc2c(C#N)cnc3cc(OCCCN4CCC(CN5C6CC5CN(c5ccc7c(c5)C(=O)N(C5CCC(=O)NC5=O)C7=O)C6)CC4)c(OC)cc23)c(Cl)cc1Cl] | 4-((2,4-dichloro-5-methoxyphenyl)amino)-7- (3-(4-((3-(2-(2,6-dioxopiperidin-3-yl)-1,3- dioxoisoindolin-5-yl)-3,6- diazabicyclo[3.1.1]heptan-6- yl)methyl)piperidin-1-yl)propoxy)-6- methoxyquinoline-3-carbonitrile |
| | 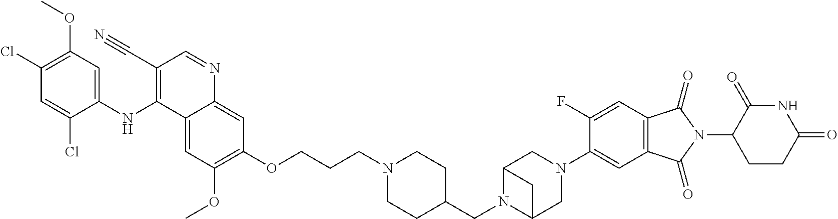 [SMILES: COc1cc(Nc2c(C#N)cnc3cc(OCCCN4CCC(CN5C6CC5CN(c5cc7c(cc5F)C(=O)N(C5CCC(=O)NC5=O)C7=O)C6)CC4)c(OC)cc23)c(Cl)cc1Cl] | 4-((2,4-dichloro-5-methoxyphenyl)amino)-7- (3-(4-((3-(2-(2,6-dioxopiperidin-3-yl)-6-fluoro- 1,3-dioxoisoindolin-5-yl)-3,6- diazabicyclo[3.1.1]heptan-6- yl)methyl)piperidin-1-yl)propoxy)-6- methoxyquinoline-3-carbonitrile |
| | 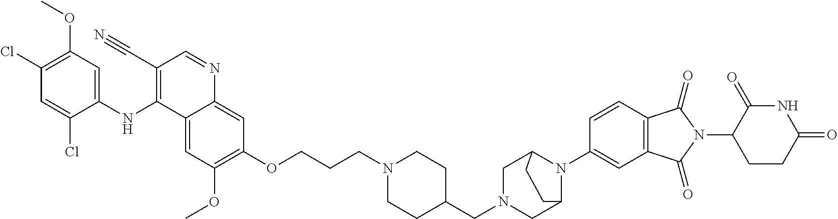 [SMILES: COc1cc(Nc2c(C#N)cnc3cc(OCCCN4CCC(CN5CC6CCC(C5)N6c5ccc6c(c5)C(=O)N(C5CCC(=O)NC5=O)C6=O)CC4)c(OC)cc23)c(Cl)cc1Cl] | 4-((2,4-dichloro-5-methoxyphenyl)amino)-7- (3-(4-((8-(2-(2,6-dioxopiperidin-3-yl)-1,3- dioxoisoindolin-5-yl)-3,8- diazabicyclo[3.2.1]octan-3-yl)methyl)piperidin- 1-yl)propoxy)-6-methoxyquinoline-3- carbonitrile |
| | 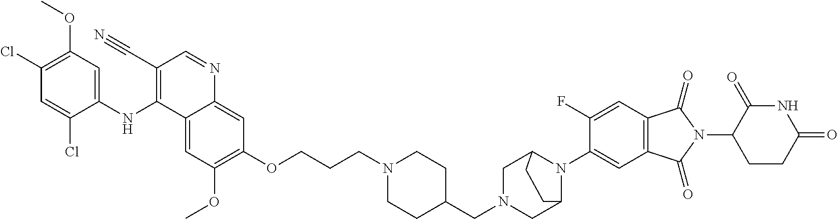 [SMILES: COc1cc(Nc2c(C#N)cnc3cc(OCCCN4CCC(CN5CC6CCC(C5)N6c5cc6c(cc5F)C(=O)N(C5CCC(=O)NC5=O)C6=O)CC4)c(OC)cc23)c(Cl)cc1Cl] | 4-((2,4-dichloro-5-methoxyphenyl)amino)-7- (3-(4-((8-(2-(2,6-dioxopiperidin-3-yl)-6-fluoro- 1,3-dioxoisoindolin-5-yl)-3,8- diazabicyclo[3.2.1]octan-3-yl)methyl)piperidin- 1-yl)propoxy)-6-methoxyquinoline-3- carbonitrile |
| | 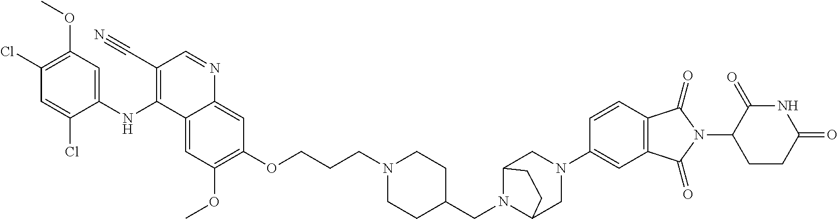 [SMILES: COc1cc(Nc2c(C#N)cnc3cc(OCCCN4CCC(CN5C6CCC5CN(c5ccc7c(c5)C(=O)N(C5CCC(=O)NC5=O)C7=O)C6)CC4)c(OC)cc23)c(Cl)cc1Cl] | 4-((2,4-dichloro-5-methoxyphenyl)amino)-7- (3-(4-((3-(2-(2,6-dioxopiperidin-3-yl)-1,3- dioxoisoindolin-5-yl)-3,8- diazabicyclo[3.2.1]octan-8-yl)methyl)piperidin- 1-yl)propoxy)-6-methoxyquinoline-3- carbonitrile |
| | 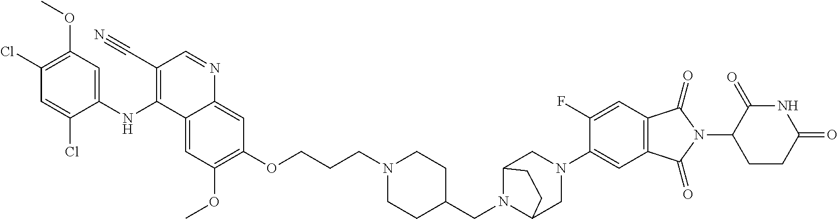 [SMILES: COc1cc(Nc2c(C#N)cnc3cc(OCCCN4CCC(CN5C6CCC5CN(c5cc7c(cc5F)C(=O)N(C5CCC(=O)NC5=O)C7=O)C6)CC4)c(OC)cc23)c(Cl)cc1Cl] | 4-((2,4-dichloro-5-methoxyphenyl)amino)-7- (3-(4-((3-(2-(2,6-dioxopiperidin-3-yl)-6-fluoro- 1,3-dioxoisoindolin-5-yl)-3,8- diazabicyclo[3.2.1]octan-8-yl)methyl)piperidin- 1-yl)propoxy)-6-methoxyquinoline-3- carbonitrile |
| | 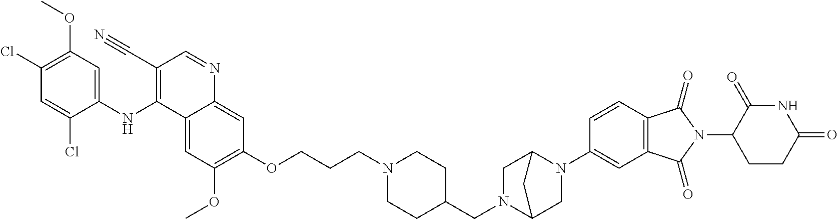 [SMILES: COc1cc(Nc2c(C#N)cnc3cc(OCCCN4CCC(CN5CC6CC5CN6c5ccc6c(c5)C(=O)N(C5CCC(=O)NC5=O)C6=O)CC4)c(OC)cc23)c(Cl)cc1Cl] | 4-((2,4-dichloro-5-methoxyphenyl)amino)-7- (3-(4-((5-(2-(2,6-dioxopiperidin-3-yl)-1,3- dioxoisoindolin-5-yl)-2,5- diazabicyclo[2.2.1]heptan-2- yl)methyl)piperidin-1-yl)propoxy)-6- methoxyquinoline-3-carbonitrile |
| | 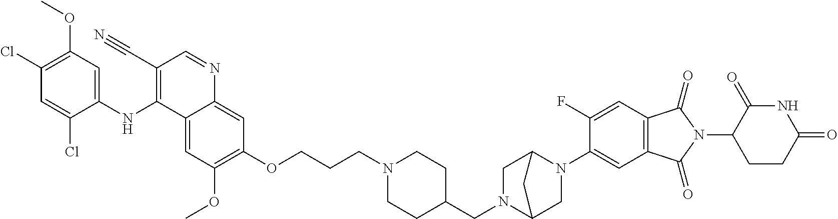 [SMILES: COc1cc(Nc2c(C#N)cnc3cc(OCCCN4CCC(CN5CC6CC5CN6c5cc6c(cc5F)C(=O)N(C5CCC(=O)NC5=O)C6=O)CC4)c(OC)cc23)c(Cl)cc1Cl] | 4-((2,4-dichloro-5-methoxyphenyl)amino)-7- (3-(4-((5-(2-(2,6-dioxopiperidin-3-yl)-6-fluoro- 1,3-dioxoisoindolin-5-yl)-2,5- diazabicyclo[2.2.1]heptan-2- yl)methyl)piperidin-1-yl)propoxy)-6- methoxyquinoline-3-carbonitrile |
| | 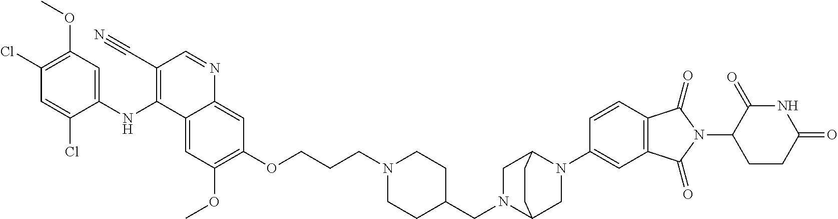 [SMILES: COc1cc(Nc2c(C#N)cnc3cc(OCCCN4CCC(CN5CC6CCC5CN6c5ccc6c(c5)C(=O)N(C5CCC(=O)NC5=O)C6=O)CC4)c(OC)cc23)c(Cl)cc1Cl] | 4-((2,4-dichloro-5-methoxyphenyl)amino)-7- (3-(4-((5-(2-(2,6-dioxopiperidin-3-yl)-1,3- dioxoisoindolin-5-yl)-2,5- diazabicyclo[2.2.2]octan-2-yl)methyl)piperidin- 1-yl)propoxy)-6-methoxyquinoline-3- carbonitrile |
| | 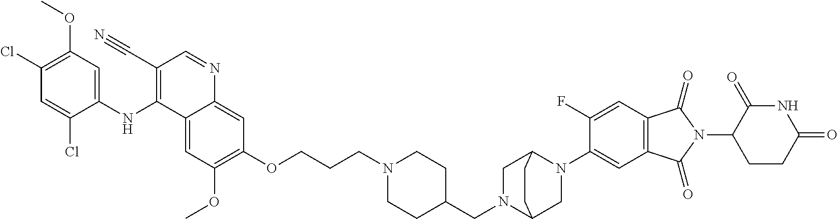 [SMILES: COc1cc(Nc2c(C#N)cnc3cc(OCCCN4CCC(CN5CC6CCC5CN6c5cc6c(cc5F)C(=O)N(C5CCC(=O)NC5=O)C6=O)CC4)c(OC)cc23)c(Cl)cc1Cl] | 4-((2,4-dichloro-5-methoxyphenyl)amino)-7- (3-(4-((5-(2-(2,6-dioxopiperidin-3-yl)-6-fluoro- 1,3-dioxoisoindolin-5-yl)-2,5- diazabicyclo[2.2.2]octan-2-yl)methyl)piperidin- 1-yl)propoxy)-6-methoxyquinoline-3- carbonitrile |
| | 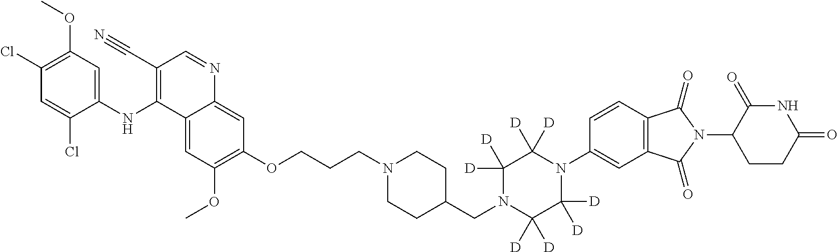 [SMILES: [2H]C1([2H])N(CC2CCN(CCCOc3cc4ncc(C#N)c(Nc5cc(OC)c(Cl)cc5Cl)c4cc3OC)CC2)C([2H])([2H])C([2H])([2H])N(c2ccc3c(c2)C(=O)N(C2CCC(=O)NC2=O)C3=O)C1([2H])[2H]] | 4-((2,4-dichloro-5-methoxyphenyl)amino)-7- (3-(4-((4-(2-(2,6-dioxopiperidin-3-yl)-1,3- dioxoisoindolin-5-yl)piperazin-1-yl- 2,2,3,3,5,5,6,6-d₈)methyl)piperidin-1- yl)propoxy)-6-methoxyquinoline-3-carbonitrile |
| | 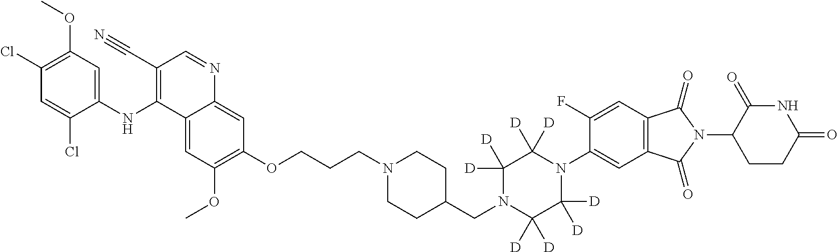 [SMILES: [2H]C1([2H])N(CC2CCN(CCCOc3cc4ncc(C#N)c(Nc5cc(OC)c(Cl)cc5Cl)c4cc3OC)CC2)C([2H])([2H])C([2H])([2H])N(c2cc3c(cc2F)C(=O)N(C2CCC(=O)NC2=O)C3=O)C1([2H])[2H]] | 4-((2,4-dichloro-5-methoxyphenyl)amino)-7- (3-(4-((4-(2-(2,6-dioxopiperidin-3-yl)-6-fluoro- 1,3-dioxoisoindolin-5-yl)piperazin-1-yl- 2,2,3,3,5,5,6,6-d₈)methyl)piperidin-1- yl)propoxy)-6-methoxyquinoline-3-carbonitrile |
| | 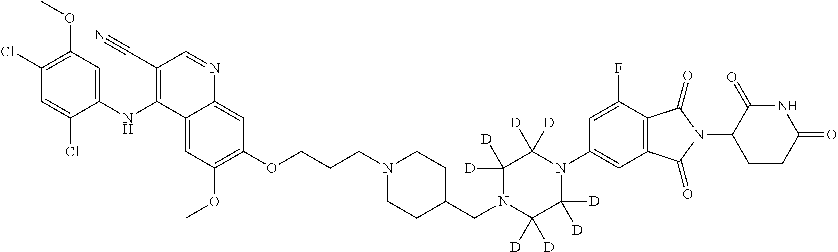 [SMILES: [2H]C1([2H])N(CC2CCN(CCCOc3cc4ncc(C#N)c(Nc5cc(OC)c(Cl)cc5Cl)c4cc3OC)CC2)C([2H])([2H])C([2H])([2H])N(c2cc(F)c3c(c2)C(=O)N(C2CCC(=O)NC2=O)C3=O)C1([2H])[2H]] | 4-((2,4-dichloro-5-methoxyphenyl)amino)-7- (3-(4-((4-(2-(2,6-dioxopiperidin-3-yl)-7-fluoro- 1,3-dioxoisoindolin-5-yl)piperazin-1-yl- 2,2,3,3,5,5,6,6-d₈)methyl)piperidin-1- yl)propoxy)-6-methoxyquinoline-3-carbonitrile |
| | 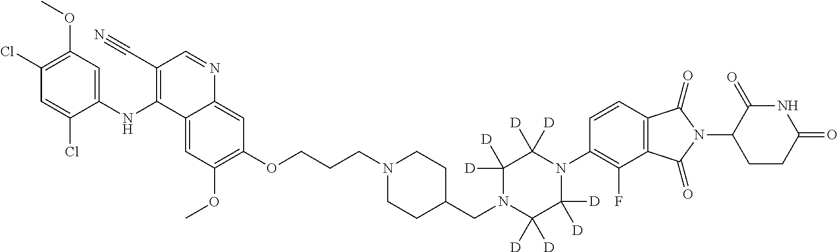 [SMILES: [2H]C1([2H])N(CC2CCN(CCCOc3cc4ncc(C#N)c(Nc5cc(OC)c(Cl)cc5Cl)c4cc3OC)CC2)C([2H])([2H])C([2H])([2H])N(c2ccc3c(c2F)C(=O)N(C2CCC(=O)NC2=O)C3=O)C1([2H])[2H]] | 4-((2,4-dichloro-5-methoxyphenyl)amino)-7- (3-(4-((4-(2-(2,6-dioxopiperidin-3-yl)-4-fluoro- 1,3-dioxoisoindolin-5-yl)piperazin-1-yl- 2,2,3,3,5,5,6,6-d;)methyl)piperidin-1- yl)propoxy)-6-methoxyquinoline-3-carbonitrile |
| | 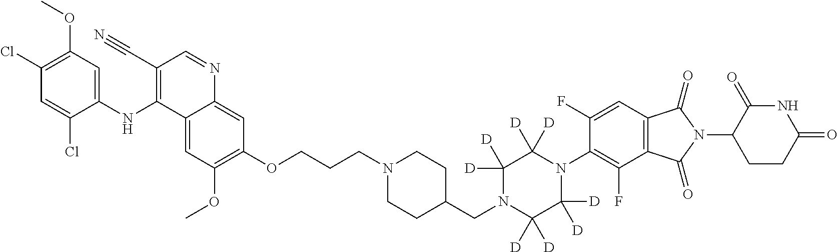 [SMILES: [2H]C1([2H])N(CC2CCN(CCCOc3cc4ncc(C#N)c(Nc5cc(OC)c(Cl)cc5Cl)c4cc3OC)CC2)C([2H])([2H])C([2H])([2H])N(c2c(F)cc3c(c2F)C(=O)N(C2CCC(=O)NC2=O)C3=O)C1([2H])[2H]] | 4-((2,4-dichloro-5-methoxyphenyl)amino)-7- (3-(4-((4-(2-(2,6-dioxopiperidin-3-yl)-4,6- difluoro-1,3-dioxoisoindolin-5-yl)piperazin-1- yl-2,2,3,3,5,5,6,6-d₈)methyl)piperidin-1- yl)propoxy)-6-methoxyquinoline-3-carbonitrile |
| | 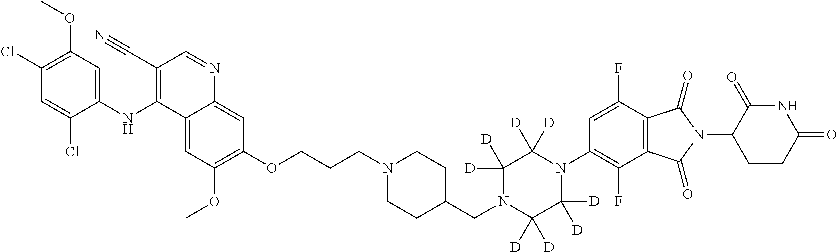 [SMILES: [2H]C1([2H])N(CC2CCN(CCCOc3cc4ncc(C#N)c(Nc5cc(OC)c(Cl)cc5Cl)c4cc3OC)CC2)C([2H])([2H])C([2H])([2H])N(c2cc(F)c3c(c2F)C(=O)N(C2CCC(=O)NC2=O)C3=O)C1([2H])[2H]] | 4-((2,4-dichloro-5-methoxyphenyl)amino)-7- (3-(4-((4-(2-(2,6-dioxopiperidin-3-yl)-4,7- difluoro-1,3-dioxoisoindolin-5-yl)piperazin-1- yl-2,2,3,3,5,5,6,6-d₈)methyl)piperidin-1- yl)propoxy)-6-methoxyquinoline-3-carbonitrile |
| | 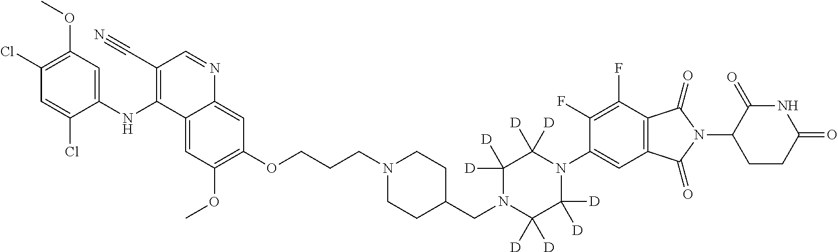 [SMILES: [2H]C1([2H])N(CC2CCN(CCCOc3cc4ncc(C#N)c(Nc5cc(OC)c(Cl)cc5Cl)c4cc3OC)CC2)C([2H])([2H])C([2H])([2H])N(c2cc3c(c(F)c2F)C(=O)N(C2CCC(=O)NC2=O)C3=O)C1([2H])[2H]] | 4-((2,4-dichloro-5-methoxyphenyl)amino)-7- (3-(4-((4-(2-(2,6-dioxopiperidin-3-yl)-6,7- difluoro-1,3-dioxoisoindolin-5-yl)piperazin-1- yl-2,2,3,3,5,5,6,6-d₈)methyl)piperidin-1- yl)propoxy)-6-methoxyquinoline-3-carbonitrile |
| | 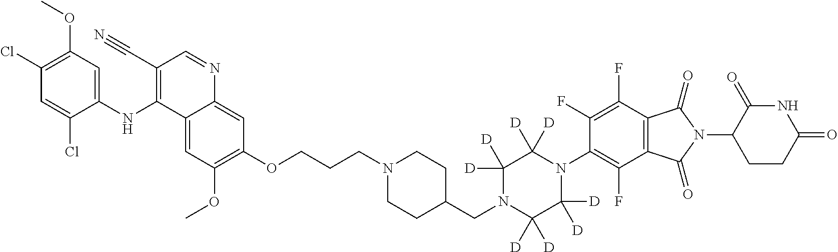 [SMILES: [2H]C1([2H])N(CC2CCN(CCCOc3cc4ncc(C#N)c(Nc5cc(OC)c(Cl)cc5Cl)c4cc3OC)CC2)C([2H])([2H])C([2H])([2H])N(c2c(F)c(F)c3c(c2F)C(=O)N(C2CCC(=O)NC2=O)C3=O)C1([2H])[2H]] | 4-((2,4-dichloro-5-methoxyphenyl)amino)-7- (3-(4-((4-(2-(2,6-dioxopiperidin-3-yl)-4,6,7- trifluoro-1,3-dioxoisoindolin-5-yl)piperazin-1 - yl-2,2,3,3,5,5,6,6-d₈)methyl)piperidin-1- yl)propoxy)-6-methoxyquinoline-3-carbonitrile |
| | 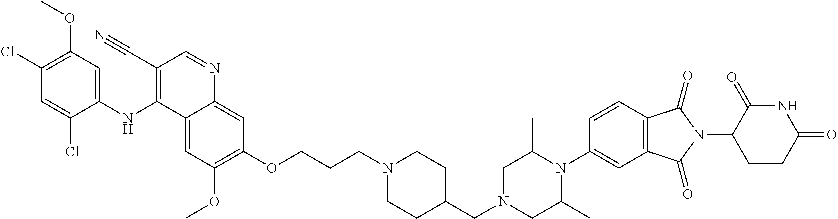 [SMILES: COc1cc(Nc2c(C#N)cnc3cc(OCCCN4CCC(CN5CC(C)N(c6ccc7c(c6)C(=O)N(C6CCC(=O)NC6=O)C7=O)C(C)C5)CC4)c(OC)cc23)c(Cl)cc1Cl] | 4-((2,4-dichloro-5-methoxyphenyl)amino)-7- (3-(4-((4-(2-(2,6-dioxopiperidin-3-yl)-1,3- dioxoisoindolin-5-yl)-3,5-dimethylpiperazin-1- yl)methyl)piperidin-1-yl)propoxy)-6- methoxyquinoline-3-carbonitrile |
| | 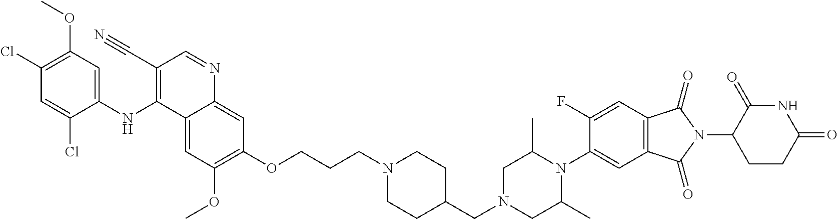 [SMILES: COc1cc(Nc2c(C#N)cnc3cc(OCCCN4CCC(CN5CC(C)N(c6cc7c(cc6F)C(=O)N(C6CCC(=O)NC6=O)C7=O)C(C)C5)CC4)c(OC)cc23)c(Cl)cc1Cl] | 4-((2,4-dichloro-5-methoxyphenyl)amino)-7- (3-(4-((4-(2-(2,6-dioxopiperidin-3-yl)-6-fluoro- 1,3-dioxoisoindolin-5-yl)-3,5- dimethylpiperazin-1-yl)methyl)piperidin-1- yl)propoxy)-6-methoxyquinoline-3-carbonitrile |
| | 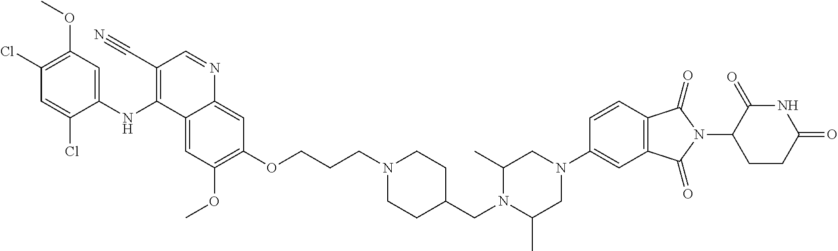 [SMILES: COc1cc(Nc2c(C#N)cnc3cc(OCCCN4CCC(CN5C(C)CN(c6ccc7c(c6)C(=O)N(C6CCC(=O)NC6=O)C7=O)CC5C)CC4)c(OC)cc23)c(Cl)cc1Cl] | 4-((2,4-dichloro-5-methoxyphenyl)amino)-7- (3-(4-((4-(2-(2,6-dioxopiperidin-3-yl)-1,3- dioxoisoindolin-5-yl)-2,6-dimethylpiperazin-1- yl)methyl)piperidin-1-yl)propoxy)-6- methoxyquinoline-3-carbonitrile |
| | 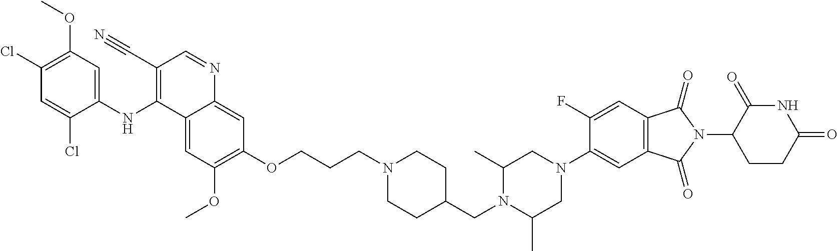 [SMILES: COc1cc(Nc2c(C#N)cnc3cc(OCCCN4CCC(CN5C(C)CN(c6cc7c(cc6F)C(=O)N(C6CCC(=O)NC6=O)C7=O)CC5C)CC4)c(OC)cc23)c(Cl)cc1Cl] | 4-((2,4-dichloro-5-methoxyphenyl)amino)-7- (3-(4-((4-(2-(2,6-dioxopiperidin-3-yl)-6-fluoro- 1,3-dioxoisoindolin-5-yl)-2,6- dimethylpiperazin-1-yl)methyl)piperidin-1- yl)propoxy)-6-methoxyquinoline-3-carbonitrile |
| | 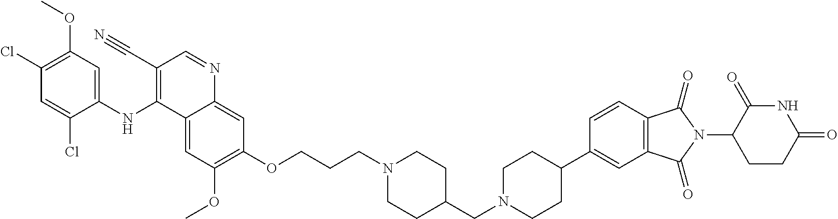 [SMILES: COc1cc(Nc2c(C#N)cnc3cc(OCCCN4CCC(CN5CCC(c6ccc7c(c6)C(=O)N(C6CCC(=O)NC6=O)C7=O)CC5)CC4)c(OC)cc23)c(Cl)cc1Cl] | 4-((2,4-dichloro-5-methoxyphenyl)amino)-7- (3-(4-((4-(2-(2,6-dioxopiperidin-3-yl)-1,3- dioxoisoindolin-5-yl)piperidin-1- yl)methyl)piperidin-1-yl)propoxy)-6- methoxyquinoline-3-carbonitrile |
| | 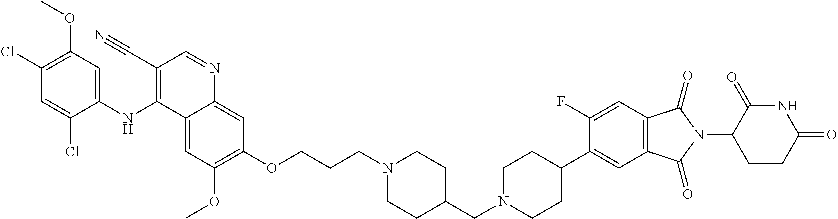 [SMILES: COc1cc(Nc2c(C#N)cnc3cc(OCCCN4CCC(CN5CCC(c6cc7c(cc6F)C(=O)N(C6CCC(=O)NC6=O)C7=O)CC5)CC4)c(OC)cc23)c(Cl)cc1Cl] | 4-((2,4-dichloro-5-methoxyphenyl)amino)-7- (3-(4-((4-(2-(2,6-dioxopiperidin-3-yl)-6-fluoro- 1,3-dioxoisoindolin-5-yl)piperidin-1- yl)methyl)piperidin-1-yl)propoxy)-6- methoxyquinoline-3-carbonitrile |
| | 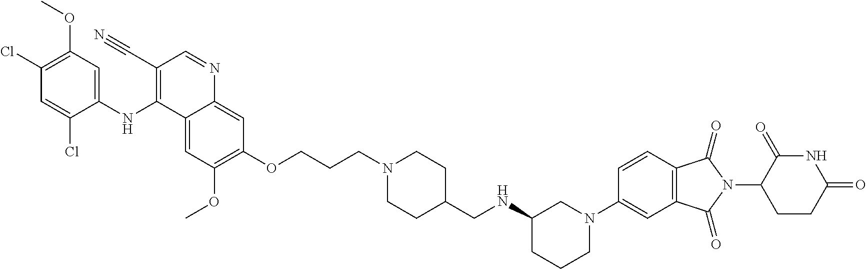 [SMILES: COc1cc(Nc2c(C#N)cnc3cc(OCCCN4CCC(CN[C@@H]5CCCN(c6ccc7c(c6)C(=O)N(C6CCC(=O)NC6=O)C7=O)C5)CC4)c(OC)cc23)c(Cl)cc1Cl] | 4-((2,4-dichloro-5-methoxyphenyl)amino)-7- (3-(4-((((3R)-1-(2-(2,6-dioxopiperidin-3-yl)- 1,3-dioxoisoindolin-5-yl)piperidin-3- yl)amino)methyl)piperidin-1-yl)propoxy)-6- methoxyquinoline-3-carbonitrile |
| | 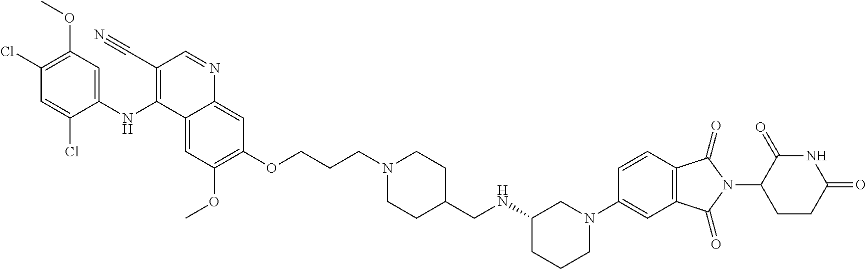 [SMILES: COc1cc(Nc2c(C#N)cnc3cc(OCCCN4CCC(CN[C@H]5CCCN(c6ccc7c(c6)C(=O)N(C6CCC(=O)NC6=O)C7=O)C5)CC4)c(OC)cc23)c(Cl)cc1Cl] | 4-((2,4-dichloro-5-methoxyphenyl)amino)-7- (3-(4-((((3S)-1-(2-(2,6-dioxopiperidin-3-yl)- 1,3-dioxoisoindolin-5-yl)piperidin-3- yl)amino)methyl)piperidin-1-yl)propoxy)-6- methoxyquinoline-3-carbonitrile |
| | 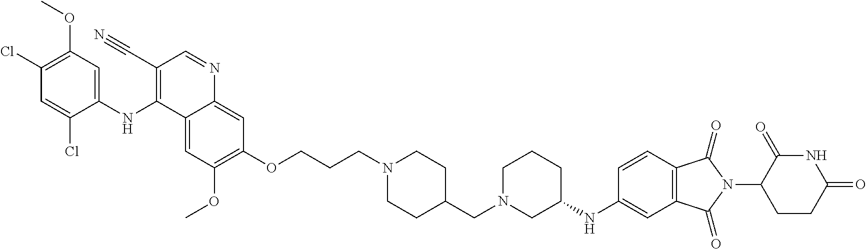 [SMILES: COc1cc(Nc2c(C#N)cnc3cc(OCCCN4CCC(CN5CCC[C@H](Nc6ccc7c(c6)C(=O)N(C6CCC(=O)NC6=O)C7=O)C5)CC4)c(OC)cc23)c(Cl)cc1Cl] | 4-((2,4-dichloro-5-methoxyphenyl)amino)-7- (3-(4-(((3S)-3-((2-(2,6-dioxopiperidin-3-yl)- 1,3-dioxoisoindolin-5-yl)amino)piperidin-1- yl)methyl)piperidin-1-yl)propoxy)-6- methoxyquinoline-3-carbonitrile |
| | 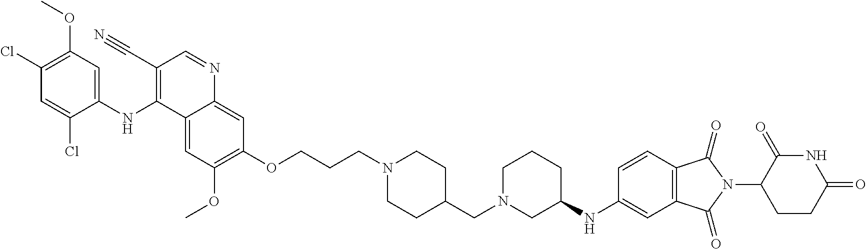 [SMILES: COc1cc(Nc2c(C#N)cnc3cc(OCCCN4CCC(CN5CCC[C@@H](Nc6ccc7c(c6)C(=O)N(C6CCC(=O)NC6=O)C7=O)C5)CC4)c(OC)cc23)c(Cl)cc1Cl] | 4-((2,4-dichloro-5-methoxyphenyl)amino)-7- (3-(4-(((3R)-3-((2-(2,6-dioxopiperidin-3-yl)- 1,3-dioxoisoindolin-5-yl)amino)piperidin-1- yl)methyl)piperidin-1-yl)propoxy)-6- methoxyquinoline-3-carbonitrile |
| | 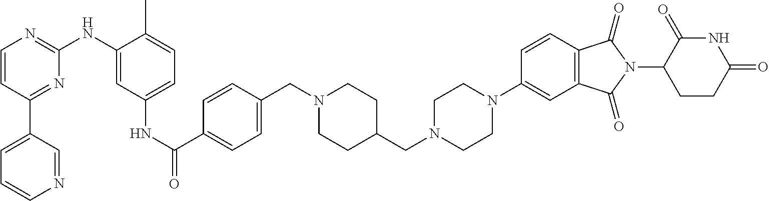 [SMILES: Cc1ccc(NC(=O)c2ccc(CN3CCC(CN4CCN(c5ccc6c(c5)C(=O)N(C5CCC(=O)NC5=O)C6=O)CC4)CC3)cc2)cc1Nc1nccc(-c2cccnc2)n1] | 4-((4-((4-(2-(2,6-dioxopiperidin-3-yl)-1,3- dioxoisoindolin-5-yl)piperazin-1- yl)methyl)piperidin-1-yl)methyl)-N-(4-methyl- 3-((4-(pyridin-3-yl)pyrimidin-2- yl)amino)phenyl)benzamide |
| | 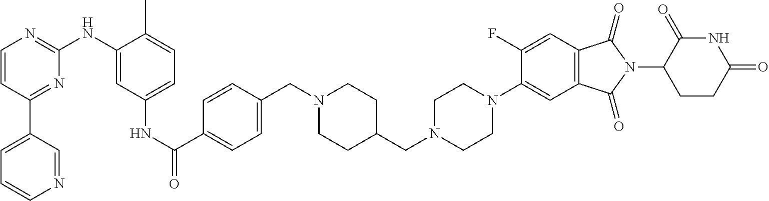 [SMILES: Cc1ccc(NC(=O)c2ccc(CN3CCC(CN4CCN(c5cc6c(cc5F)C(=O)N(C5CCC(=O)NC5=O)C6=O)CC4)CC3)cc2)cc1Nc1nccc(-c2cccnc2)n1] | 4-((4-((4-(2-(2,6-dioxopiperidin-3-yl)-6-fluoro- 1,3-dioxoisoindolin-5-yl)piperazin-1- yl)methyl)piperidin-1-yl)methyl)-N-(4-methyl- 3-((4-(pyridin-3-yl)pyrimidin-2- yl)amino)phenyl)benzamide |
| | 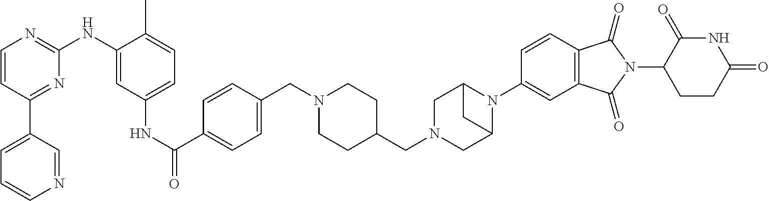 [SMILES: Cc1ccc(NC(=O)c2ccc(CN3CCC(CN4CC5CC(C4)N5c4ccc5c(c4)C(=O)N(C4CCC(=O)NC4=O)C5=O)CC3)cc2)cc1Nc1nccc(-c2cccnc2)n1] | 4-((4-((6-(2-(2,6-dioxopiperidin-3-yl)-1,3- dioxoisoindolin-5-yl)-3,6- diazabicyclo[3.1.1]heptan-3- yl)methyl)piperidin-1-yl)methyl)-N-(4-methyl- 3-((4-(pyridin-3-yl)pyrimidin-2- yl)amino)phenyl)benzamide |
| | 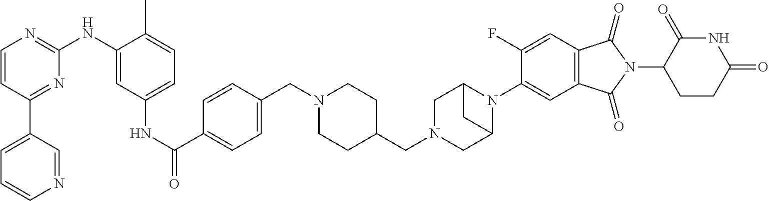 [SMILES: Cc1ccc(NC(=O)c2ccc(CN3CCC(CN4CC5CC(C4)N5c4cc5c(cc4F)C(=O)N(C4CCC(=O)NC4=O)C5=O)CC3)cc2)cc1Nc1nccc(-c2cccnc2)n1] | 4-((4-((6-(2-(2,6-dioxopiperidin-3-yl)-6-fluoro- 1,3-dioxoisoindolin-5-yl)-3,6- diazabicyclo[3.1.1]heptan-3- yl)methyl)piperidin-1-yl)methyl)-N-(4-methyl- 3-((4-(pyridin-3-yl)pyrimidin-2- yl)amino)phenyl)benzamide |
| | 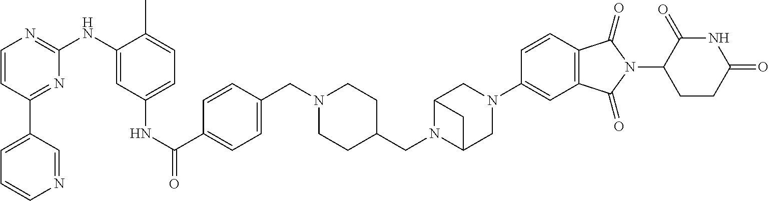 [SMILES: Cc1ccc(NC(=O)c2ccc(CN3CCC(CN4C5CC4CN(c4ccc6c(c4)C(=O)N(C4CCC(=O)NC4=O)C6=O)C5)CC3)cc2)cc1Nc1nccc(-c2cccnc2)n1] | 4-((4-((3-(2-(2,6-dioxopiperidin-3-yl)-1,3- dioxoisoindolin-5-yl)-3,6- diazabicyclo[3.1.1]heptan-6- yl)methyl)piperidin-1-yl)methyl)-N-(4-methyl- 3-((4-(pyridin-3-yl)pyrimidin-2- yl)amino)phenyl)benzamide |
| | 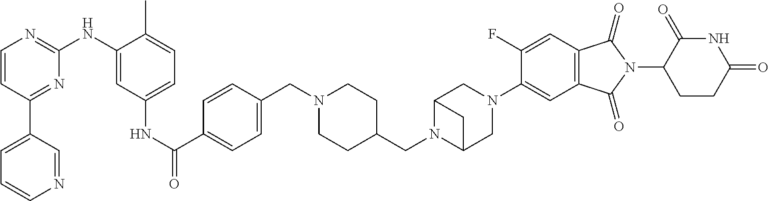 [SMILES: Cc1ccc(NC(=O)c2ccc(CN3CCC(CN4C5CC4CN(c4cc6c(cc4F)C(=O)N(C4CCC(=O)NC4=O)C6=O)C5)CC3)cc2)cc1Nc1nccc(-c2cccnc2)n1] | 4-((4-((3-(2-(2,6-dioxopiperidin-3-yl)-6-fluoro- 1,3-dioxoisoindolin-5-yl)-3,6- diazabicyclo[3.1.1]heptan-6- yl)methyl)piperidin-1-yl)methyl)-N-(4-methyl- 3-((4-(pyridin-3-yl)pyrimidin-2- yl)amino)phenyl)benzamide |
| | 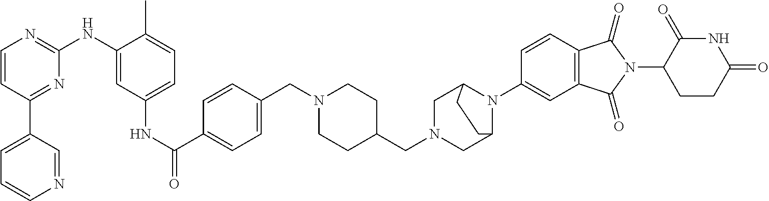 [SMILES: Cc1ccc(NC(=O)c2ccc(CN3CCC(CN4CC5CCC(C4)N5c4ccc5c(c4)C(=O)N(C4CCC(=O)NC4=O)C5=O)CC3)cc2)cc1Nc1nccc(-c2cccnc2)n1] | 4-((4-((8-(2-(2,6-dioxopiperidin-3-yl)-1,3- dioxoisoindolin-5-yl)-3,8- diazabicyclo[3.2.1]octan-3-yl)methyl)piperidin- 1-yl)methyl)-N-(4-methyl-3-((4-(pyridin-3- yl)pyrimidin-2-yl)amino)phenyl)benzamide |
| | 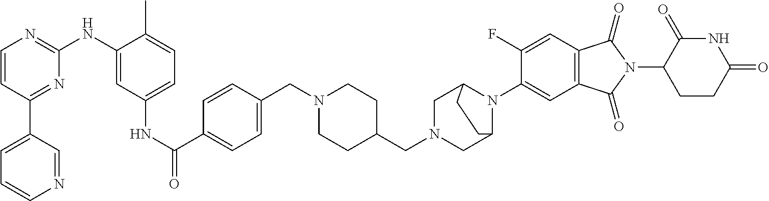 [SMILES: Cc1ccc(NC(=O)c2ccc(CN3CCC(CN4CC5CCC(C4)N5c4cc5c(cc4F)C(=O)N(C4CCC(=O)NC4=O)C5=O)CC3)cc2)cc1Nc1nccc(-c2cccnc2)n1] | 4-((4-((8-(2-(2,6-dioxopiperidin-3-yl)-6-fluoro- 1,3-dioxoisoindolin-5-yl)-3,8- diazabicyclo[3.2.1]octan-3-yl)methyl)piperidin- 1-yl)methyl)-N-(4-methyl-3-((4-(pyridin-3- yl)pyrimidin-2-yl)amino)phenyl)benzamide |
| | 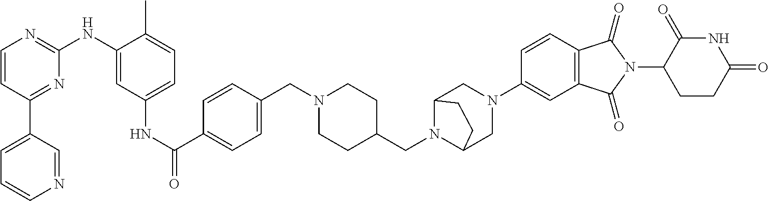 [SMILES: Cc1ccc(NC(=O)c2ccc(CN3CCC(CN4C5CCC4CN(c4ccc6c(c4)C(=O)N(C4CCC(=O)NC4=O)C6=O)C5)CC3)cc2)cc1Nc1nccc(-c2cccnc2)n1] | 4-((4-((3-(2-(2,6-dioxopiperidin-3-yl)-1,3- dioxoisoindolin-5-yl)-3,8- diazabicyclo[3.2.1]octan-8-yl)methyl)piperidin- 1-yl)methyl)-N-(4-methyl-3-((4-(pyridin-3- yl)pyrimidin-2-yl)amino)phenyl)benzamide |
| | 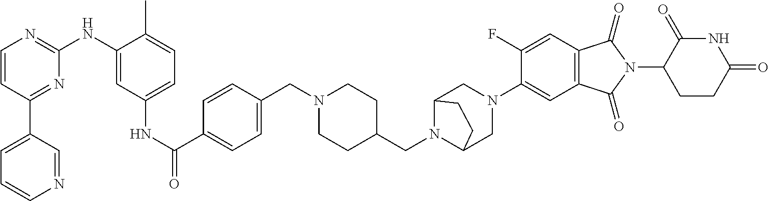 [SMILES: Cc1ccc(NC(=O)c2ccc(CN3CCC(CN4C5CCC4CN(c4cc6c(cc4F)C(=O)N(C4CCC(=O)NC4=O)C6=O)C5)CC3)cc2)cc1Nc1nccc(-c2cccnc2)n1] | 4-((4-((3-(2-(2,6-dioxopiperidin-3-yl)-6-fluoro- 1,3-dioxoisoindolin-5-yl)-3,8- diazabicyclo[3.2.1]octan-8-yl)methyl)piperidin- 1-yl)methyl)-N-(4-methyl-3-((4-(pyridin-3- yl)pyrimidin-2-yl)amino)phenyl)benzamide |
| | 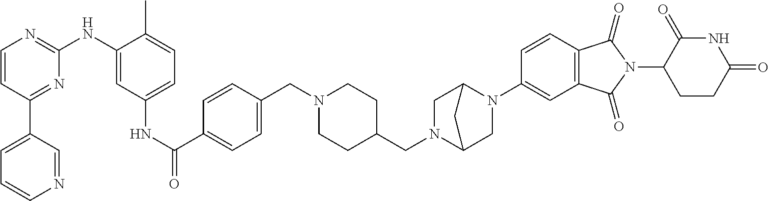 [SMILES: Cc1ccc(NC(=O)c2ccc(CN3CCC(CN4CC5CC4CN5c4ccc5c(c4)C(=O)N(C4CCC(=O)NC4=O)C5=O)CC3)cc2)cc1Nc1nccc(-c2cccnc2)n1] | 4-((4-((5-(2-(2,6-dioxopiperidin-3-yl)-1,3- dioxoisoindolin-5-yl)-2,5- diazabicyclo[2.2.1]heptan-2- yl)methyl)piperidin-1-yl)methyl)-N-(4-methyl- 3-((4-(pyridin-3-yl)pyrimidin-2- yl)amino)phenyl)benzamide |
| | 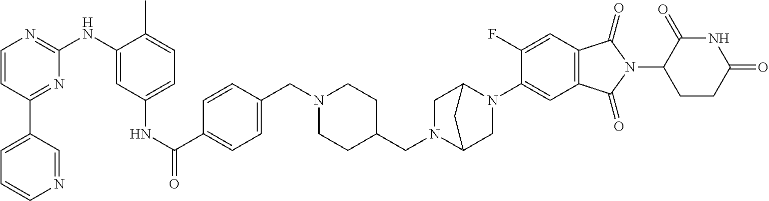 [SMILES: Cc1ccc(NC(=O)c2ccc(CN3CCC(CN4CC5CC4CN5c4cc5c(cc4F)C(=O)N(C4CCC(=O)NC4=O)C5=O)CC3)cc2)cc1Nc1nccc(-c2cccnc2)n1] | 4-((4-((5-(2-(2,6-dioxopiperidin-3-yl)-6-fluoro- 1,3-dioxoisoindolin-5-yl)-2,5- diazabicyclo[2.2.1]heptan-2- yl)methyl)piperidin-1-yl)methyl)-N-(4-methyl- 3-((4-(pyridin-3-yl)pyrimidin-2- yl)amino)phenyl)benzamide |
| | 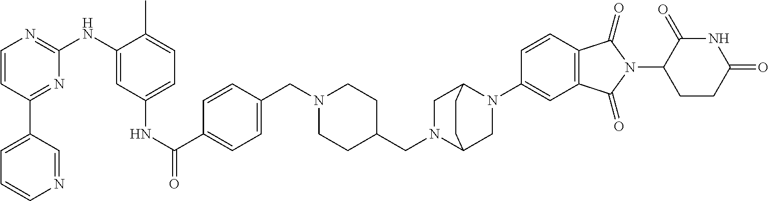 [SMILES: Cc1ccc(NC(=O)c2ccc(CN3CCC(CN4CC5CCC4CN5c4ccc5c(c4)C(=O)N(C4CCC(=O)NC4=O)C5=O)CC3)cc2)cc1Nc1nccc(-c2cccnc2)n1] | 4-((4-((5-(2-(2,6-dioxopiperidin-3-yl)-1,3- dioxoisoindolin-5-yl)-2,5- diazabicyclo[2.2.2]octan-2-yl)methyl)piperidin- 1-yl)methyl)-N-(4-methyl-3-((4-(pyridin-3- yl)pyrimidin-2-yl)amino)phenyl)benzamide |
| | 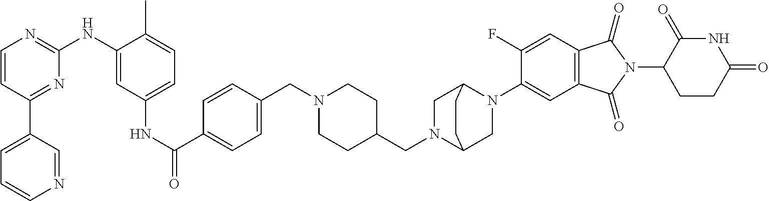 [SMILES: Cc1ccc(NC(=O)c2ccc(CN3CCC(CN4CC5CCC4CN5c4cc5c(cc4F)C(=O)N(C4CCC(=O)NC4=O)C5=O)CC3)cc2)cc1Nc1nccc(-c2cccnc2)n1] | 4-((4-((5-(2-(2,6-dioxopiperidin-3-yl)-6-fluoro- 1,3-dioxoisoindolin-5-yl)-2,5- diazabicyclo[2.2.2]octan-2-yl)methyl)piperidin- 1-yl)methyl)-N-(4-methyl-3-((4-(pyridin-3- yl)pyrimidin-2-yl)amino)phenyl)benzamide |
| | 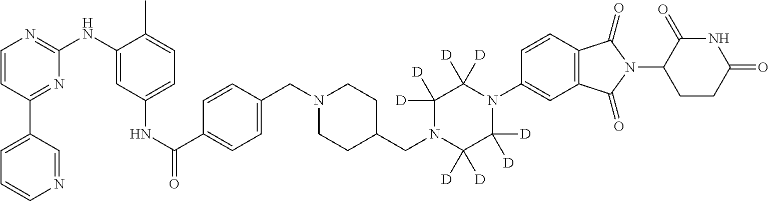 [SMILES: [2H]C1([2H])N(CC2CCN(Cc3ccc(C(=O)Nc4ccc(C)c(Nc5nccc(-c6cccnc6)n5)c4)cc3)CC2)C([2H])([2H])C([2H])([2H])N(c2ccc3c(c2)C(=O)N(C2CCC(=O)NC2=O)C3=O)C1([2H])[2H]] | 4-((4-((4-(2-(2,6-dioxopiperidin-3-yl)-1,3- dioxoisoindolin-5-yl)piperazin-1-yl- 2,2,3,3,5,5,6,6-d₈)methyl)piperidin-1- yl)methyl)-N-(4-methyl-3-((4-(pyridin-3- yl)pyrimidin-2-yl)amino)phenyl)benzamide |
| | 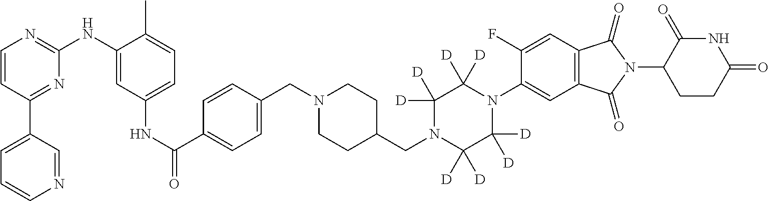 [SMILES: [2H]C1([2H])N(CC2CCN(Cc3ccc(C(=O)Nc4ccc(C)c(Nc5nccc(-c6cccnc6)n5)c4)cc3)CC2)C([2H])([2H])C([2H])([2H])N(c2cc3c(cc2F)C(=O)N(C2CCC(=O)NC2=O)C3=O)C1([2H])[2H]] | 4-((4-((4-(2-(2,6-dioxopiperidin-3-yl)-6-fluoro- 1,3-dioxoisoindolin-5-yl)piperazin-1-yl- 2,2,3,3,5,5,6,6-d₈)methyl)piperidin-1- yl)methyl)-N-(4-methyl-3-((4-(pyridin-3- yl)pyrimidin-2-yl)amino)phenyl)benzamide |
| | 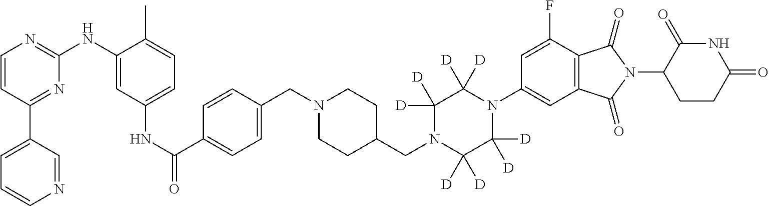 [SMILES: [2H]C1([2H])N(CC2CCN(Cc3ccc(C(=O)Nc4ccc(C)c(Nc5nccc(-c6cccnc6)n5)c4)cc3)CC2)C([2H])([2H])C([2H])([2H])N(c2cc(F)c3c(c2)C(=O)N(C2CCC(=O)NC2=O)C3=O)C1([2H])[2H]] | 4-((4-((4-(2-(2,6-dioxopiperidin-3-yl)-7-fluoro- 1,3-dioxoisoindolin-5-yl)piperazin-1-yl- 2,2,3,3,5,5,6,6-d₈)methyl)piperidin-1- yl)methyl)-N-(4-methyl-3-((4-(pyridin-3- yl)pyrimidin-2-yl)amino)phenyl)benzamide |
| | 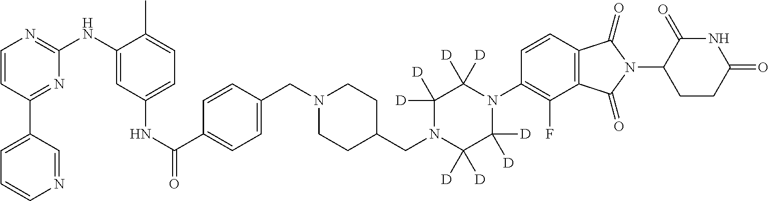 [SMILES: [2H]C1([2H])N(CC2CCN(Cc3ccc(C(=O)Nc4ccc(C)c(Nc5nccc(-c6cccnc6)n5)c4)cc3)CC2)C([2H])([2H])C([2H])([2H])N(c2ccc3c(c2F)C(=O)N(C2CCC(=O)NC2=O)C3=O)C1([2H])[2H]] | 4-((4-((4-(2-(2,6-dioxopiperidin-3-yl)-4-fluoro- 1,3-dioxoisoindolin-5-yl)piperazin-1-yl- 2,2,3,3,5,5,6,6-d₈)methyl)piperidin-1- yl)methyl)-N-(4-methyl-3-((4-(pyridin-3- yl)pyrimidin-2-yl)amino)phenyl)benzamide |
| | 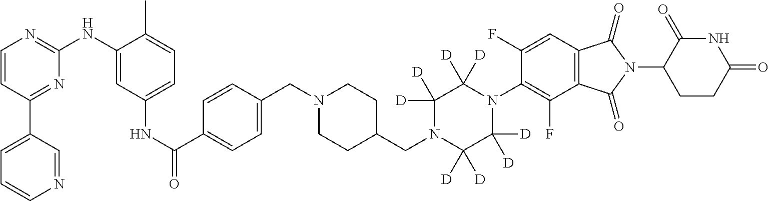 [SMILES: [2H]C1([2H])N(CC2CCN(Cc3ccc(C(=O)Nc4ccc(C)c(Nc5nccc(-c6cccnc6)n5)c4)cc3)CC2)C([2H])([2H])C([2H])([2H])N(c2c(F)cc3c(c2F)C(=O)N(C2CCC(=O)NC2=O)C3=O)C1([2H])[2H]] | 4-((4-((4-(2-(2,6-dioxopiperidin-3-yl)-4,6- difluoro-1,3-dioxoisoindolin-5-yl)piperazin-1- yl-2,2,3,3,5,5,6,6-d₈)methyl)piperidin-1- yl)methyl)-N-(4-methyl-3-((4-(pyridin-3- yl)pyrimidin-2-yl)amino)phenyl)benzamide |
| | 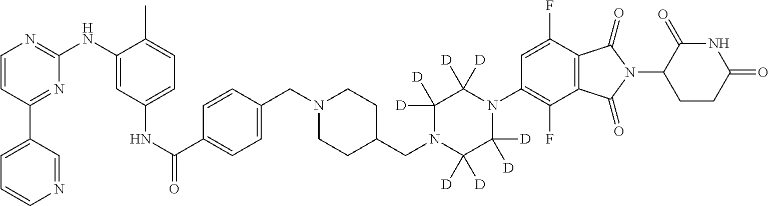 [SMILES: [2H]C1([2H])N(CC2CCN(Cc3ccc(C(=O)Nc4ccc(C)c(Nc5nccc(-c6cccnc6)n5)c4)cc3)CC2)C([2H])([2H])C([2H])([2H])N(c2cc(F)c3c(c2F)C(=O)N(C2CCC(=O)NC2=O)C3=O)C1([2H])[2H]] | 4-((4-((4-(2-(2,6-dioxopiperidin-3-yl)-4,7- difluoro-1,3-dioxoisoindolin-5-yl)piperazin-1- yl-2,2,3,3,5,5,6,6-d₈)methyl)piperidin-1- yl)methyl)-N-(4-methyl-3-((4-(pyridin-3- yl)pyrimidin-2-yl)amino)phenyl)benzamide |
| | 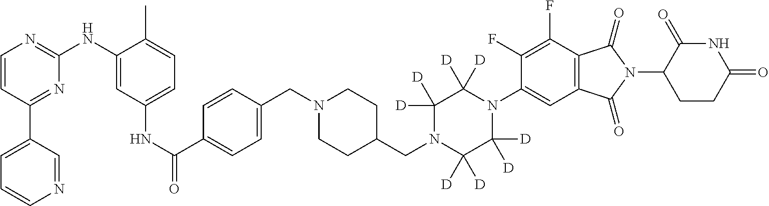 [SMILES: [2H]C1([2H])N(CC2CCN(Cc3ccc(C(=O)Nc4ccc(C)c(Nc5nccc(-c6cccnc6)n5)c4)cc3)CC2)C([2H])([2H])C([2H])([2H])N(c2cc3c(c(F)c2F)C(=O)N(C2CCC(=O)NC2=O)C3=O)C1([2H])[2H]] | 4-((4-((4-(2-(2,6-dioxopiperidin-3-yl)-6,7- difluoro-1,3-dioxoisoindolin-5-yl)piperazin-1- yl-2,2,3,3,5,5,6,6-d₈)methyl)piperidin-1- yl)methyl)-N-(4-methyl-3-((4-(pyridin-3- yl)pyrimidin-2-yl)amino)phenyl)benzamide |
| | 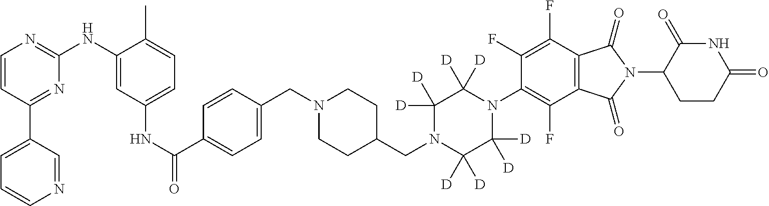 [SMILES: [2H]C1([2H])N(CC2CCN(Cc3ccc(C(=O)Nc4ccc(C)c(Nc5nccc(-c6cccnc6)n5)c4)cc3)CC2)C([2H])([2H])C([2H])([2H])N(c2c(F)c(F)c3c(c2F)C(=O)N(C2CCC(=O)NC2=O)C3=O)C1([2H])[2H]] | 4-((4-((4-(2-(2,6-dioxopiperidin-3-yl)-4,6,7- trifluoro-1,3-dioxoisoindolin-5-yl)piperazin-1- yl-2,2,3,3,5,5,6,6-d₈)methyl)piperidin-1- yl)methyl)-N-(4-methyl-3-((4-(pyridin-3- yl)pyrimidin-2-yl)amino)phenyl)benzamide |
| | 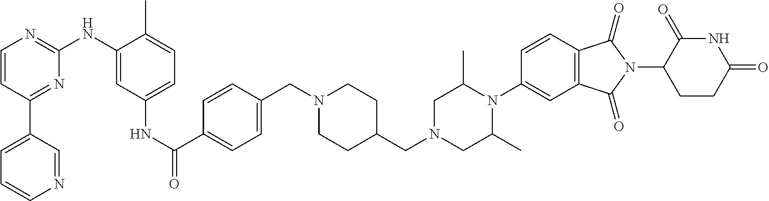 [SMILES: Cc1ccc(NC(=O)c2ccc(CN3CCC(CN4CC(C)N(c5ccc6c(c5)C(=O)N(C5CCC(=O)NC5=O)C6=O)C(C)C4)CC3)cc2)cc1Nc1nccc(-c2cccnc2)n1] | 4-((4-((4-(2-(2,6-dioxopiperidin-3-yl)-1,3- dioxoisoindolin-5-yl)-3,5-dimethylpiperazin-1- yl)methyl)piperidin-1-yl)methyl)-N-(4-methyl- 3-((4-(pyridin-3-yl)pyrimidin-2- yl)amino)phenyl)benzamide |
| | 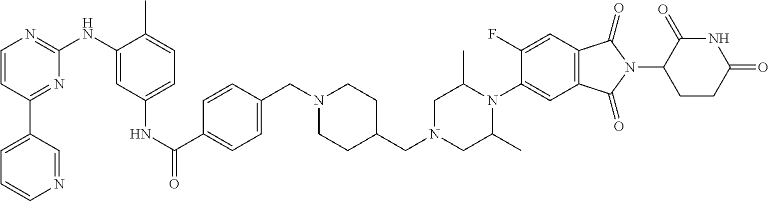 [SMILES: Cc1ccc(NC(=O)c2ccc(CN3CCC(CN4CC(C)N(c5cc6c(cc5F)C(=O)N(C5CCC(=O)NC5=O)C6=O)C(C)C4)CC3)cc2)cc1Nc1nccc(-c2cccnc2)n1] | 4-((4-((4-(2-(2,6-dioxopiperidin-3-yl)-6-fluoro- 1,3-dioxoisoindolin-5-yl)-3,5- dimethylpiperazin-1-yl)methyl)piperidin-1- yl)methyl)-N-(4-methyl-3-((4-(pyridin-3- yl)pyrimidin-2-yl)amino)phenyl)benzamide |
| | 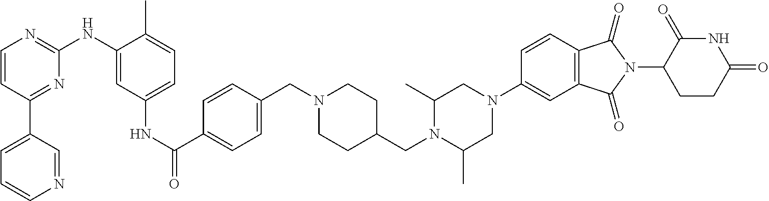 [SMILES: Cc1ccc(NC(=O)c2ccc(CN3CCC(CN4C(C)CN(c5ccc6c(c5)C(=O)N(C5CCC(=O)NC5=O)C6=O)CC4C)CC3)cc2)cc1Nc1nccc(-c2cccnc2)n1] | 4-((4-((4-(2-(2,6-dioxopiperidin-3-yl)-1,3- dioxoisoindolin-5-yl)-2,6-dimethylpiperazin-1- yl)methyl)piperidin-1-yl)methyl)-N-(4-methyl- 3-((4-(pyridin-3-yl)pyrimidin-2- yl)amino)phenyl)benzamide |
| | 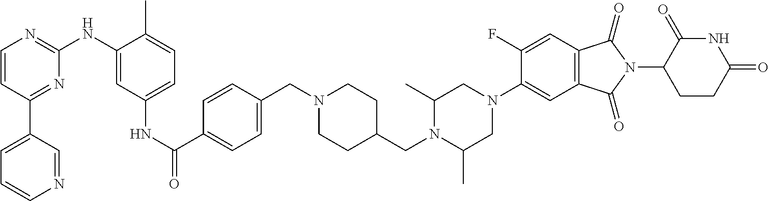 [SMILES: Cc1ccc(NC(=O)c2ccc(CN3CCC(CN4C(C)CN(c5cc6c(cc5F)C(=O)N(C5CCC(=O)NC5=O)C6=O)CC4C)CC3)cc2)cc1Nc1nccc(-c2cccnc2)n1] | 4-((4-((4-(2-(2,6-dioxopiperidin-3-yl)-6-fluoro- 1,3-dioxoisoindolin-5-yl)-2,6- dimethylpiperazin-1-yl)methyl)piperidin-1- yl)methyl)-N-(4-methyl-3-((4-(pyridin-3- yl)pyrimidin-2-yl)amino)phenyl)benzamide |
| | 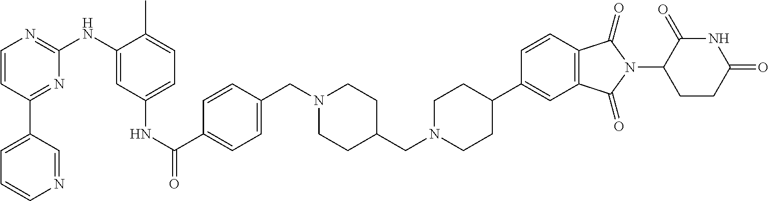 [SMILES: Cc1ccc(NC(=O)c2ccc(CN3CCC(CN4CCC(c5ccc6c(c5)C(=O)N(C5CCC(=O)NC5=O)C6=O)CC4)CC3)cc2)cc1Nc1nccc(-c2cccnc2)n1] | 4-((4-((4-(2-(2,6-dioxopiperidin-3-yl)-1,3- dioxoisoindolin-5-yl)piperidin-1- yl)methyl)piperidin-1-yl)methyl)-N-(4-methyl- 3-((4-(pyridin-3-yl)pyrimidin-2- yl)amino)phenyl)benzamide |
| | 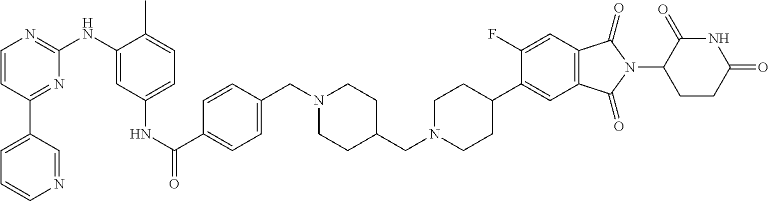 [SMILES: Cc1ccc(NC(=O)c2ccc(CN3CCC(CN4CCC(c5cc6c(cc5F)C(=O)N(C5CCC(=O)NC5=O)C6=O)CC4)CC3)cc2)cc1Nc1nccc(-c2cccnc2)n1] | 4-((4-((4-(2-(2,6-dioxopiperidin-3-yl)-6-fluoro- 1,3-dioxoisoindolin-5-yl)piperidin-1- yl)methyl)piperidin-1-yl)methyl)-N-(4-methyl- 3-((4-(pyridin-3-yl)pyrimidin-2- yl)amino)phenyl)benzamide |
| | 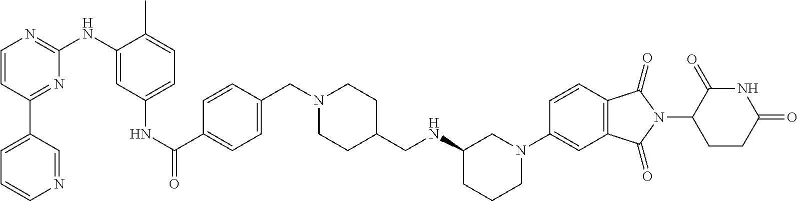 [SMILES: Cc1ccc(NC(=O)c2ccc(CN3CCC(CN[C@@H]4CCCN(c5ccc6c(c5)C(=O)N(C5CCC(=O)NC5=O)C6=O)C4)CC3)cc2)cc1Nc1nccc(-c2cccnc2)n1] | 4-((4-((((3R)-1-(2-(2,6-dioxopiperidin-3-yl)- 1,3-dioxoisoindolin-5-yl)piperidin-3- yl)amino)methyl)piperidin-1-yl)methyl)-N-(4- methyl-3-((4-(pyridin-3-yl)pyrimidin-2- yl)amino)phenyl)benzamide |
| | 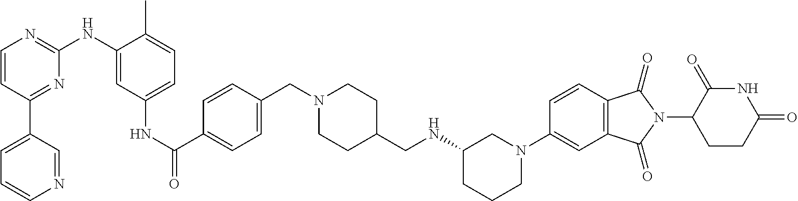 [SMILES: Cc1ccc(NC(=O)c2ccc(CN3CCC(CN[C@H]4CCCN(c5ccc6c(c5)C(=O)N(C5CCC(=O)NC5=O)C6=O)C4)CC3)cc2)cc1Nc1nccc(-c2cccnc2)n1] | 4-((4-((((3S)-1-(2-(2,6-dioxopiperidin-3-yl)- 1,3-dioxoisoindolin-5-yl)piperidin-3- yl)amino)methyl)piperidin-1-yl)methyl)-N-(4- methyl-3-((4-(pyridin-3-yl)pyrimidin-2- yl)amino)phenyl)benzamide |
| | 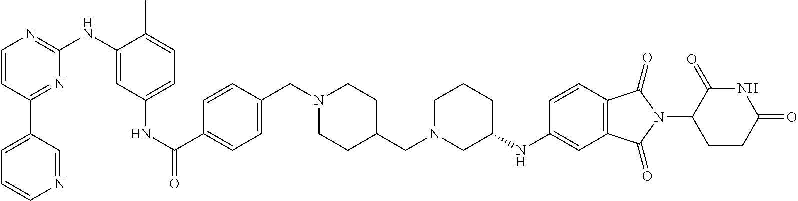 [SMILES: Cc1ccc(NC(=O)c2ccc(CN3CCC(CN4CCC[C@H](Nc5ccc6c(c5)C(=O)N(C5CCC(=O)NC5=O)C6=O)C4)CC3)cc2)cc1Nc1nccc(-c2cccnc2)n1] | 4-((4-(((3S)-3-((2-(2,6-dioxopiperidin-3-yl)- 1,3-dioxoisoindolin-5-yl)amino)piperidin-1- yl)methyl)piperidin-1-yl)methyl)-N-(4-methyl- 3-((4-(pyridin-3-yl)pyrimidin-2- yl)amino)phenyl)benzamide |
| | 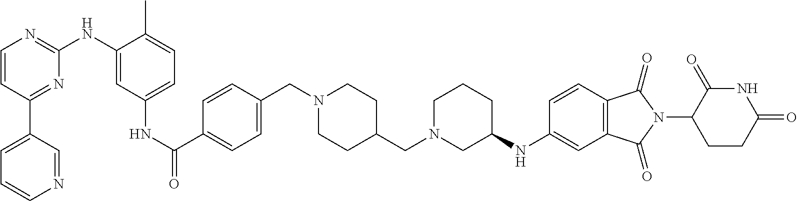 [SMILES: Cc1ccc(NC(=O)c2ccc(CN3CCC(CN4CCC[C@@H](Nc5ccc6c(c5)C(=O)N(C5CCC(=O)NC5=O)C6=O)C4)CC3)cc2)cc1Nc1nccc(-c2cccnc2)n1] | 4-((4-(((3R)-3-((2-(2,6-dioxopiperidin-3-yl)- 1,3-dioxoisoindolin-5-yl)amino)piperidin-1- yl)methyl)piperidin-1-yl)methyl)-N-(4-methyl- 3-((4-(pyridin-3-yl)pyrimidin-2- yl)amino)phenyl)benzamide |
| | 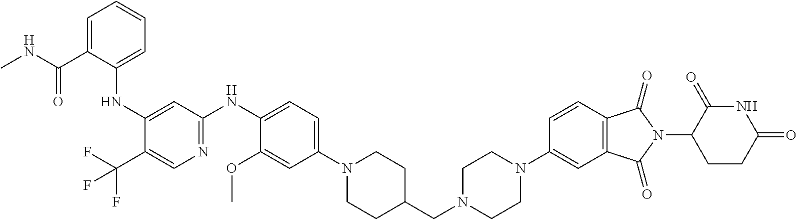 [SMILES: CNC(=O)c1ccccc1Nc1cc(Nc2ccc(N3CCC(CN4CCN(c5ccc6c(c5)C(=O)N(C5CCC(=O)NC5=O)C6=O)CC4)CC3)cc2OC)ncc1C(F)(F)F] | 2-((2-((4-(4-((4-(2-(2,6-dioxopiperidin-3-yl)- 1,3-dioxoisoindolin-5-yl)piperazin-1- yl)methyl)piperidin-1-yl)-2- methoxyphenyl)amino)-5- (trifluoromethyl)pyridin-4-yl)amino)-N- methylbenzamide |
| | 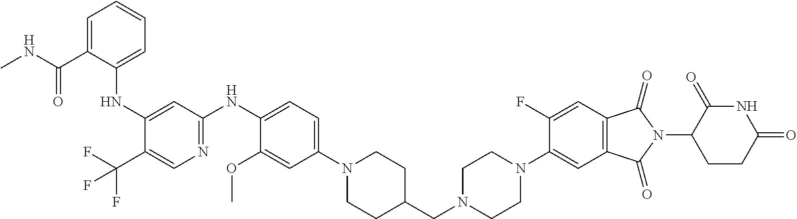 [SMILES: CNC(=O)c1ccccc1Nc1cc(Nc2ccc(N3CCC(CN4CCN(c5cc6c(cc5F)C(=O)N(C5CCC(=O)NC5=O)C6=O)CC4)CC3)cc2OC)ncc1C(F)(F)F] | 2-((2-((4-(4-((4-(2-(2,6-dioxopiperidin-3-yl)-6- fluoro-1,3-dioxoisoindolin-5-yl)piperazin-1- yl)methyl)piperidin-1-yl)-2- methoxyphenyl)amino)-5- (trifluoromethyl)pyridin-4-yl)amino)-N- methylbenzamide |
| | 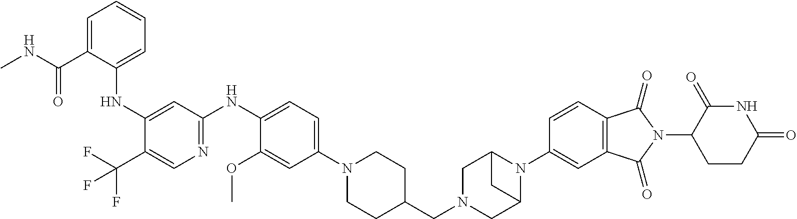 [SMILES: CNC(=O)c1ccccc1Nc1cc(Nc2ccc(N3CCC(CN4CC5CC(C4)N5c4ccc5c(c4)C(=O)N(C4CCC(=O)NC4=O)C5=O)CC3)cc2OC)ncc1C(F)(F)F] | 2-((2-((4-(4-((6-(2-(2,6-dioxopiperidin-3-yl)- 1,3-dioxoisoindolin-5-yl)-3,6- diazabicyclo[3.1.1]heptan-3- yl)methyl)piperidin-1-yl)-2- methoxyphenyl)amino)-5- (trifluoromethyl)pyridin-4-yl)amino)-N- methylbenzamide |
| | 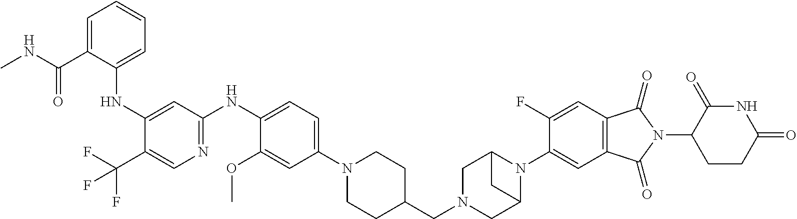 [SMILES: CNC(=O)c1ccccc1Nc1cc(Nc2ccc(N3CCC(CN4CC5CC(C4)N5c4cc5c(cc4F)C(=O)N(C4CCC(=O)NC4=O)C5=O)CC3)cc2OC)ncc1C(F)(F)F] | 2-((2-((4-(4-((6-(2-(2,6-dioxopiperidin-3-yl)-6- fluoro-1,3-dioxoisoindolin-5-yl)-3,6- diazabicyclo[3.1.1]heptan-3- yl)methyl)piperidin-1-yl)-2- methoxyphenyl)amino)-5- (trifluoromethyl)pyridin-4-yl)amino)-N- methylbenzamide |
| | 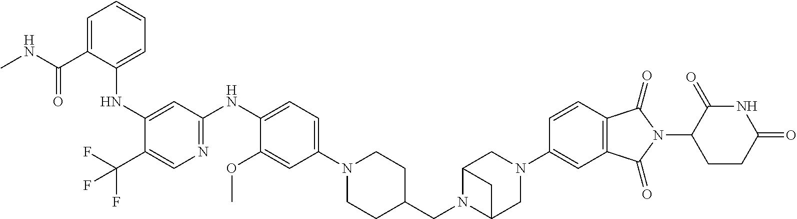 [SMILES: CNC(=O)c1ccccc1Nc1cc(Nc2ccc(N3CCC(CN4C5CC4CN(c4ccc6c(c4)C(=O)N(C4CCC(=O)NC4=O)C6=O)C5)CC3)cc2OC)ncc1C(F)(F)F] | 2-((2-((4-(4-((3-(2-(2,6-dioxopiperidin-3-yl)- 1,3-dioxoisoindolin-5-yl)-3,6- diazabicyclo[3.1.1]heptan-6- yl)methyl)piperidin-1-yl)-2- methoxyphenyl)amino)-5- (trifluoromethyl)pyridin-4-yl)amino)-N- methylbenzamide |
| | 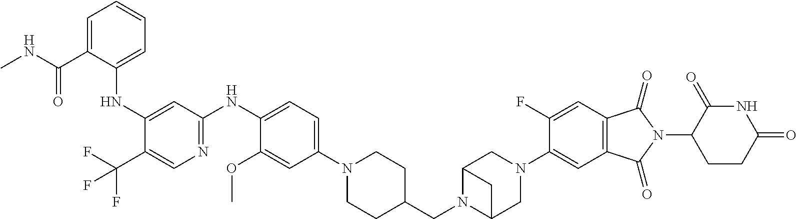 [SMILES: CNC(=O)c1ccccc1Nc1cc(Nc2ccc(N3CCC(CN4C5CC4CN(c4cc6c(cc4F)C(=O)N(C4CCC(=O)NC4=O)C6=O)C5)CC3)cc2OC)ncc1C(F)(F)F] | 2-((2-((4-(4-((3-(2-(2,6-dioxopiperidin-3-yl)-6- fluoro-1,3-dioxoisoindolin-5-yl)-3,6- diazabicyclo[3.1.1]heptan-6- yl)methyl)piperidin-1-yl)-2- methoxyphenyl)amino)-5- (trifluoromethyl)pyridin-4-yl)amino)-N- methylbenzamide |
| | 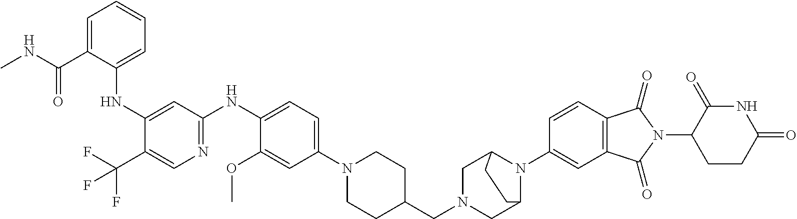 [SMILES: CNC(=O)c1ccccc1Nc1cc(Nc2ccc(N3CCC(CN4CC5CCC(C4)N5c4ccc5c(c4)C(=O)N(C4CCC(=O)NC4=O)C5=O)CC3)cc2OC)ncc1C(F)(F)F] | 2-((2-((4-(4-((8-(2-(2,6-dioxopiperidin-3-yl)- 1,3-dioxoisoindolin-5-yl)-3,8- diazabicyclo[3.2.1]octan-3-yl)methyl)piperidin- 1-yl)-2-methoxyphenyl)amino)-5- (trifluoromethyl)pyridin-4-yl)amino)-N- methylbenzamide |
| | 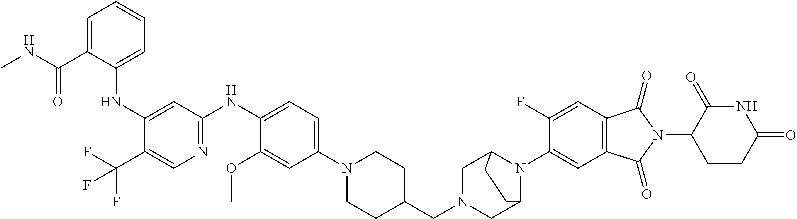 [SMILES: CNC(=O)c1ccccc1Nc1cc(Nc2ccc(N3CCC(CN4CC5CCC(C4)N5c4cc5c(cc4F)C(=O)N(C4CCC(=O)NC4=O)C5=O)CC3)cc2OC)ncc1C(F)(F)F] | 2-((2-((4-(4-((8-(2-(2,6-dioxopiperidin-3-yl)-6- fluoro-1,3-dioxoisoindolin-5-yl)-3,8- diazabicyclo[3.2.1]octan-3-yl)methyl)piperidin- 1-yl)-2-methoxyphenyl)amino)-5- (trifluoromethyl)pyridin-4-yl)amino)-N- methylbenzamide |
| | 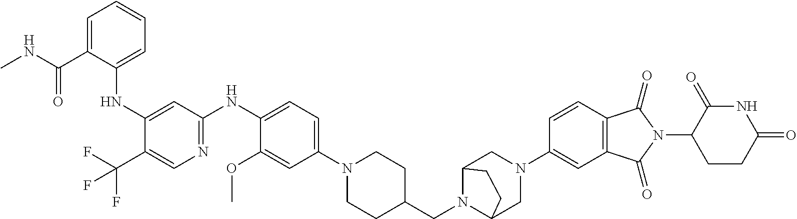 [SMILES: CNC(=O)c1ccccc1Nc1cc(Nc2ccc(N3CCC(CN4C5CCC4CN(c4ccc6c(c4)C(=O)N(C4CCC(=O)NC4=O)C6=O)C5)CC3)cc2OC)ncc1C(F)(F)F] | 2-((2-((4-(4-((3-(2-(2,6-dioxopiperidin-3-yl)- 1,3-dioxoisoindolin-5-yl)-3,8- diazabicyclo[3.2.1]octan-8-yl)methyl)piperidin- 1-yl)-2-methoxyphenyl)amino)-5- (trifluoromethyl)pyridin-4-yl)amino)-N- methylbenzamide |
| | 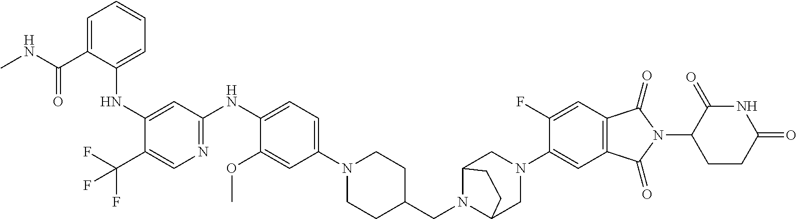 [SMILES: CNC(=O)c1ccccc1Nc1cc(Nc2ccc(N3CCC(CN4C5CCC4CN(c4cc6c(cc4F)C(=O)N(C4CCC(=O)NC4=O)C6=O)C5)CC3)cc2OC)ncc1C(F)(F)F] | 2-((2-((4-(4-((3-(2-(2,6-dioxopiperidin-3-yl)-6- fluoro-1,3-dioxoisoindolin-5-yl)-3,8- diazabicyclo[3.2.1]octan-8-yl)methyl)piperidin- 1-yl)-2-methoxyphenyl)amino)-5- (trifluoromethyl)pyridin-4-yl)amino)-N- methylbenzamide |
| | 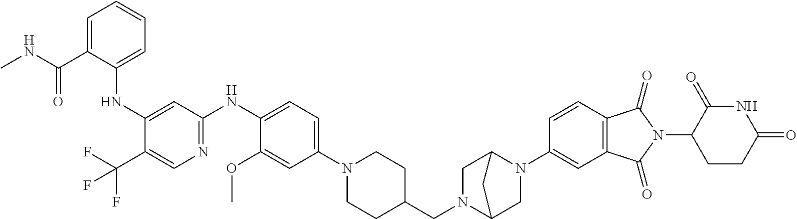 [SMILES: CNC(=O)c1ccccc1Nc1cc(Nc2ccc(N3CCC(CN4CC5CC4CN5c4ccc5c(c4)C(=O)N(C4CCC(=O)NC4=O)C5=O)CC3)cc2OC)ncc1C(F)(F)F] | 2-((2-((4-(4-((5-(2-(2,6-dioxopiperidin-3-yl)- 1,3-dioxoisoindolin-5-yl)-2,5- diazabicyclo[2.2.1]heptan-2- yl)methyl)piperidin-1-yl)-2- methoxyphenyl)amino)-5- (trifluoromethyl)pyridin-4-yl)amino)-N- methylbenzamide |
| | 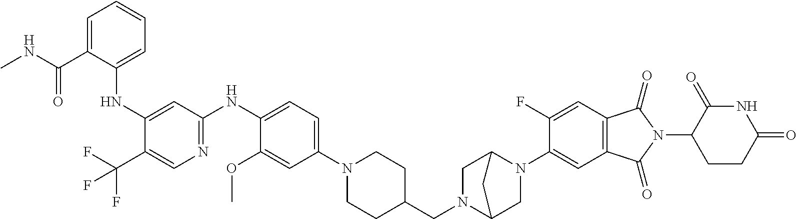 [SMILES: CNC(=O)c1ccccc1Nc1cc(Nc2ccc(N3CCC(CN4CC5CC4CN5c4cc5c(cc4F)C(=O)N(C4CCC(=O)NC4=O)C5=O)CC3)cc2OC)ncc1C(F)(F)F] | 2-((2-((4-(4-((5-(2-(2,6-dioxopiperidin-3-yl)-6- fluoro-1,3-dioxoisoindolin-5-yl)-2,5- diazabicyclo[2.2.1]heptan-2- yl)methyl)piperidin-1-yl)-2- methoxyphenyl)amino)-5- (trifluoromethyl)pyridin-4-yl)amino)-N- methylbenzamide |
| | 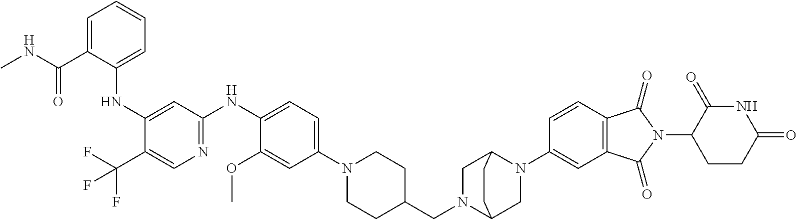 [SMILES: CNC(=O)c1ccccc1Nc1cc(Nc2ccc(N3CCC(CN4CC5CCC4CN5c4ccc5c(c4)C(=O)N(C4CCC(=O)NC4=O)C5=O)CC3)cc2OC)ncc1C(F)(F)F] | 2-((2-((4-(4-((5-(2-(2,6-dioxopiperidin-3-yl)- 1,3-dioxoisoindolin-5-yl)-2,5- diazabicyclo[2.2.2]octan-2-yl)methyl)piperidin- 1-yl)-2-methoxyphenyl)amino)-5- (trifluoromethyl)pyridin-4-yl)amino)-N- methylbenzamide |
| | 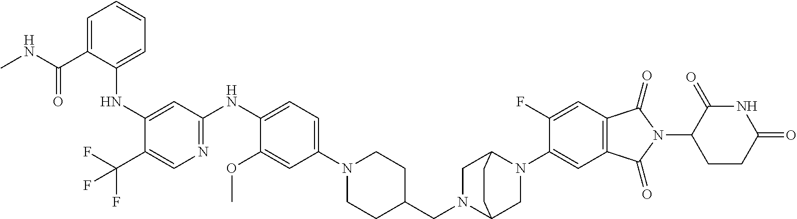 [SMILES: CNC(=O)c1ccccc1Nc1cc(Nc2ccc(N3CCC(CN4CC5CCC4CN5c4cc5c(cc4F)C(=O)N(C4CCC(=O)NC4=O)C5=O)CC3)cc2OC)ncc1C(F)(F)F] | 2-((2-((4-(4-((5-(2-(2,6-dioxopiperidin-3-yl)-6- fluoro-1,3-dioxoisoindolin-5-yl)-2,5- diazabicyclo[2.2.2]octan-2-yl)methyl)piperidin- 1-yl)-2-methoxyphenyl)amino)-5- (trifluoromethyl)pyridin-4-yl)amino)-N- methylbenzamide |
| | 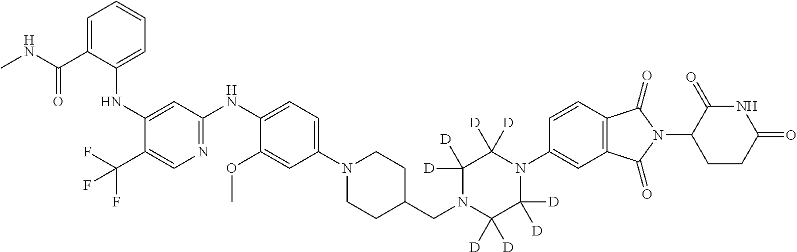 [SMILES: [2H]C1([2H])N(CC2CCN(c3ccc(Nc4cc(Nc5ccccc5C(=O)NC)c(C(F)(F)F)cn4)c(OC)c3)CC2)C([2H])([2H])C([2H])([2H])N(c2ccc3c(c2)C(=O)N(C2CCC(=O)NC2=O)C3=O)C1([2H])[2H]] | 2-((2-((4-(4-((4-(2-(2,6-dioxopiperidin-3-yl)- 1,3-dioxoisoindolin-5-yl)piperazin-1-yl- 2,2,3,3,5,5,6,6-d₈)methyl)piperidin-1-yl)-2- methoxyphenyl)amino)-5- (trifluoromethyl)pyridin-4-yl)amino)-N- methylbenzamide |
| | 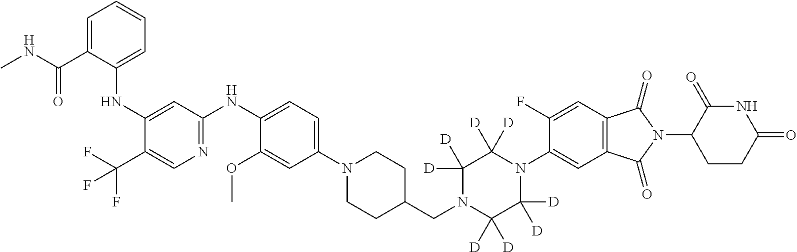 [SMILES: [2H]C1([2H])N(CC2CCN(c3ccc(Nc4cc(Nc5ccccc5C(=O)NC)c(C(F)(F)F)cn4)c(OC)c3)CC2)C([2H])([2H])C([2H])([2H])N(c2cc3c(cc2F)C(=O)N(C2CCC(=O)NC2=O)C3=O)C1([2H])[2H]] | 2-((2-((4-(4-((4-(2-(2,6-dioxopiperidin-3-yl)-6- fluoro-1,3-dioxoisoindolin-5-yl)piperazin-1-yl- 2,2,3,3,5,5,6,6-d₈)methyl)piperidin-1-yl)-2- methoxyphenyl)amino)-5- (trifluoromethyl)pyridin-4-yl)amino)-N- methylbenzamide |
| | 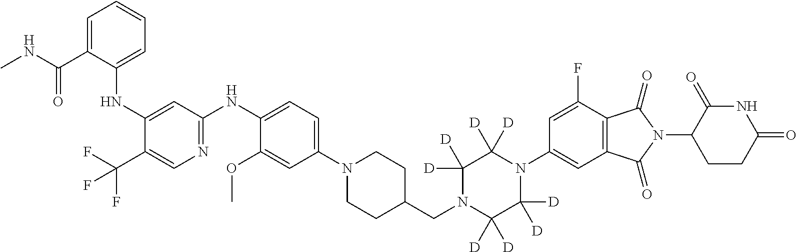 [SMILES: [2H]C1([2H])N(CC2CCN(c3ccc(Nc4cc(Nc5ccccc5C(=O)NC)c(C(F)(F)F)cn4)c(OC)c3)CC2)C([2H])([2H])C([2H])([2H])N(c2cc(F)c3c(c2)C(=O)N(C2CCC(=O)NC2=O)C3=O)C1([2H])[2H]] | 2-((2-((4-(4-((4-(2-(2,6-dioxopiperidin-3-yl)-7- fluoro-1,3-dioxoisoindolin-5-yl)piperazin-1-yl- 2,2,3,3,5,5,6,6-d₈)methyl)piperidin-1-yl)-2- methoxyphenyl)amino)-5- (trifluoromethyl)pyridin-4-yl)amino)-N- methylbenzamide |
| | 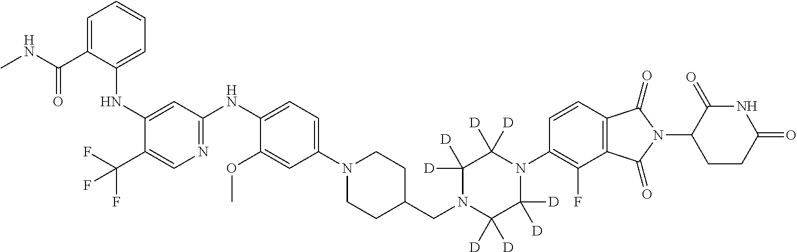 [SMILES: [2H]C1([2H])N(CC2CCN(c3ccc(Nc4cc(Nc5ccccc5C(=O)NC)c(C(F)(F)F)cn4)c(OC)c3)CC2)C([2H])([2H])C([2H])([2H])N(c2ccc3c(c2F)C(=O)N(C2CCC(=O)NC2=O)C3=O)C1([2H])[2H]] | 2-((2-((4-(4-((4-(2-(2,6-dioxopiperidin-3-yl)-4- fluoro-1,3-dioxoisoindolin-5-yl)piperazin-1-yl- 2,2,3,3,5,5,6,6-d₈)methyl)piperidin-1-yl)-2- methoxyphenyl)amino)-5- (trifluoromethyl)pyridin-4-yl)amino)-N- methylbenzamide |
| | 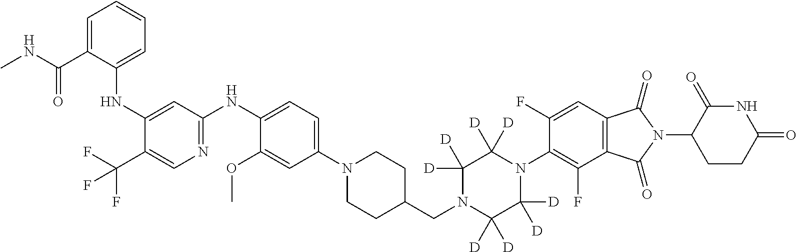 [SMILES: [2H]C1([2H])N(CC2CCN(c3ccc(Nc4cc(Nc5ccccc5C(=O)NC)c(C(F)(F)F)cn4)c(OC)c3)CC2)C([2H])([2H])C([2H])([2H])N(c2c(F)cc3c(c2F)C(=O)N(C2CCC(=O)NC2=O)C3=O)C1([2H])[2H]] | 2-((2-((4-(4-((4-(2-(2,6-dioxopiperidin-3-yl)- 4,6-difluoro-1,3-dioxoisoindolin-5- yl)piperazin-1-yl-2,2,3,3,5,5,6,6- d₈)methyl)piperidin-1-yl)-2- methoxyphenyl)amino)-5- (trifluoromethyl)pyridin-4-yl)amino)-N- methylbenzamide |
| | 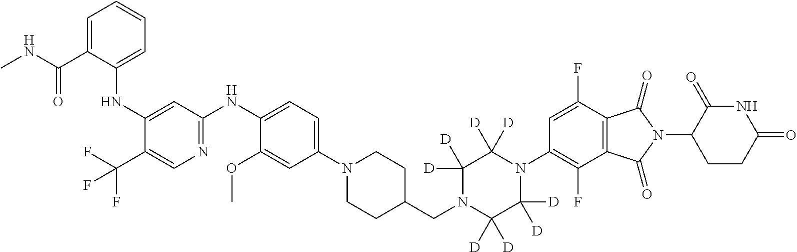 [SMILES: [2H]C1([2H])N(CC2CCN(c3ccc(Nc4cc(Nc5ccccc5C(=O)NC)c(C(F)(F)F)cn4)c(OC)c3)CC2)C([2H])([2H])C([2H])([2H])N(c2cc(F)c3c(c2F)C(=O)N(C2CCC(=O)NC2=O)C3=O)C1([2H])[2H]] | 2-((2-((4-(4-((4-(2-(2,6-dioxopiperidin-3-yl)- 4,7-difluoro-1,3-dioxoisoindolin-5- yl)piperazin-1-yl-2,2,3,3,5,5,6,6- d₈)methyl)piperidin-1-yl)-2- methoxyphenyl)amino)-5- (trifluoromethyl)pyridin-4-yl)amino)-N- methylbenzamide |
| | 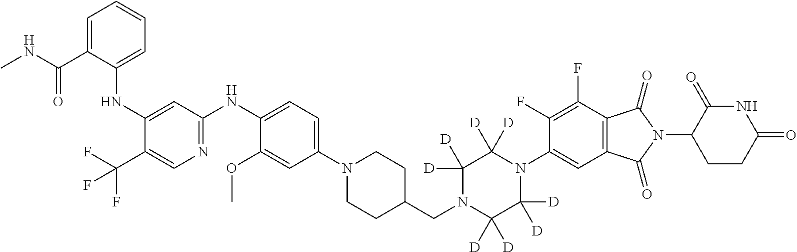 [SMILES: [2H]C1([2H])N(CC2CCN(c3ccc(Nc4cc(Nc5ccccc5C(=O)NC)c(C(F)(F)F)cn4)c(OC)c3)CC2)C([2H])([2H])C([2H])([2H])N(c2cc3c(c(F)c2F)C(=O)N(C2CCC(=O)NC2=O)C3=O)C1([2H])[2H]] | 2-((2-((4-(4-((4-(2-(2,6-dioxopiperidin-3-yl)- 6,7-difluoro-1,3-dioxoisoindolin-5- yl)piperazin-1-yl-2,2,3,3,5,5,6,6- d₈)methyl)piperidin-1-yl)-2- methoxyphenyl)amino)-5- (trifluoromethyl)pyridin-4-yl)amino)-N- methylbenzamide |
| | 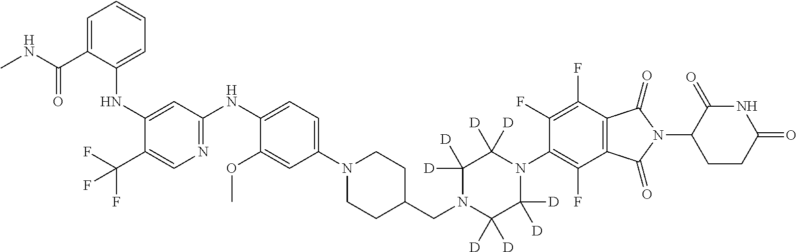 [SMILES: [2H]C1([2H])N(CC2CCN(c3ccc(Nc4cc(Nc5ccccc5C(=O)NC)c(C(F)(F)F)cn4)c(OC)c3)CC2)C([2H])([2H])C([2H])([2H])N(c2c(F)c(F)c3c(c2F)C(=O)N(C2CCC(=O)NC2=O)C3=O)C1([2H])[2H]] | 2-((2-((4-(4-((4-(2-(2,6-dioxopiperidin-3-yl)- 4,6,7-trifluoro-1,3-dioxoisoindolin-5- yl)piperazin-1-yl-2,2,3,3,5,5,6,6- d₈)methyl)piperidin-1-yl)-2- methoxyphenyl)amino)-5- (trifluoromethyl)pyridin-4-yl)amino)-N- methylbenzamide |
| | 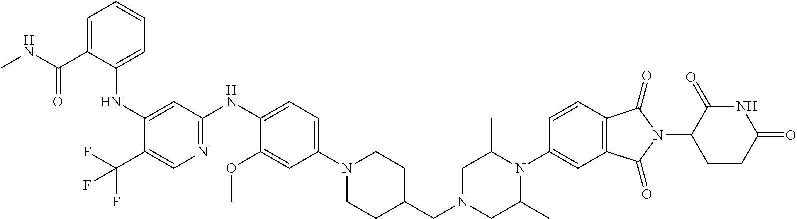 [SMILES: CNC(=O)c1ccccc1Nc1cc(Nc2ccc(N3CCC(CN4CC(C)N(c5ccc6c(c5)C(=O)N(C5CCC(=O)NC5=O)C6=O)C(C)C4)CC3)cc2OC)ncc1C(F)(F)F] | 2-((2-((4-(4-((4-(2-(2,6-dioxopiperidin-3-yl)- 1,3-dioxoisoindolin-5-yl)-3,5- dimethylpiperazin-1-yl)methyl)piperidin-1-yl)- 2-methoxyphenyl)amino)-5- (trifluoromethyl)pyridin-4-yl)amino)-N- methylbenzamide |
| | 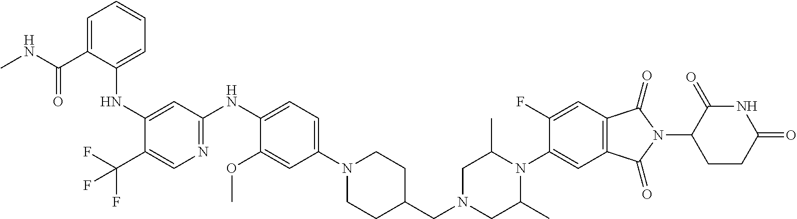 [SMILES: CNC(=O)c1ccccc1Nc1cc(Nc2ccc(N3CCC(CN4CC(C)N(c5cc6c(cc5F)C(=O)N(C5CCC(=O)NC5=O)C6=O)C(C)C4)CC3)cc2OC)ncc1C(F)(F)F] | 2-((2-((4-(4-((4-(2-(2,6-dioxopiperidin-3-yl)-6- fluoro-1,3-dioxoisoindolin-5-yl)-3,5- dimethylpiperazin-1-yl)methyl)piperidin-1-yl)- 2-methoxyphenyl)amino)-5- (trifluoromethyl)pyridin-4-yl)amino)-N- methylbenzamide |
| | 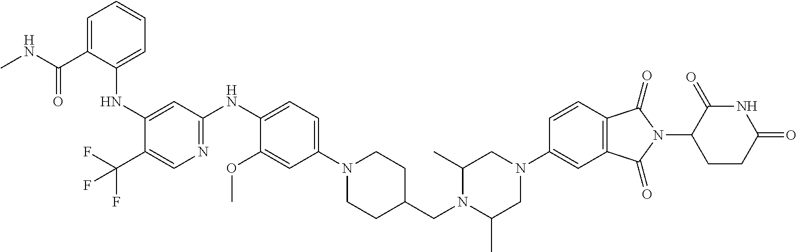 [SMILES: CNC(=O)c1ccccc1Nc1cc(Nc2ccc(N3CCC(CN4C(C)CN(c5ccc6c(c5)C(=O)N(C5CCC(=O)NC5=O)C6=O)CC4C)CC3)cc2OC)ncc1C(F)(F)F] | 2-((2-((4-(4-((4-(2-(2,6-dioxopiperidin-3-yl)- 1,3-dioxoisoindolin-5-yl)-2,6- dimethylpiperazin-1-yl)methyl)piperidin-1-yl)- 2-methoxyphenyl)amino)-5- (trifluoromethyl)pyridin-4-yl)amino)-N- methylbenzamide |
| | 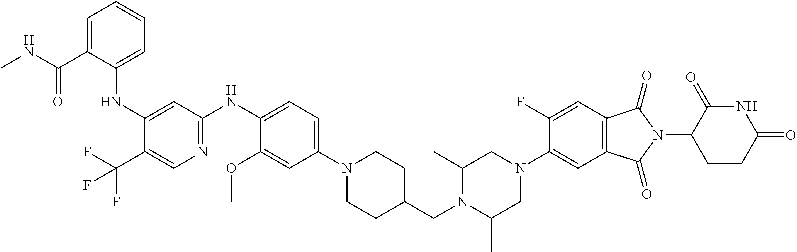 [SMILES: CNC(=O)c1ccccc1Nc1cc(Nc2ccc(N3CCC(CN4C(C)CN(c5cc6c(cc5F)C(=O)N(C5CCC(=O)NC5=O)C6=O)CC4C)CC3)cc2OC)ncc1C(F)(F)F] | 2-((2-((4-(4-((4-(2-(2,6-dioxopiperidin-3-yl)-6- fluoro-1,3-dioxoisoindolin-5-yl)-2,6- dimethylpiperazin-1-yl)methyl)piperidin-1-yl)- 2-methoxyphenyl)amino)-5- (trifluoromethyl)pyridin-4-yl)amino)-N- methylbenzamide |
| | 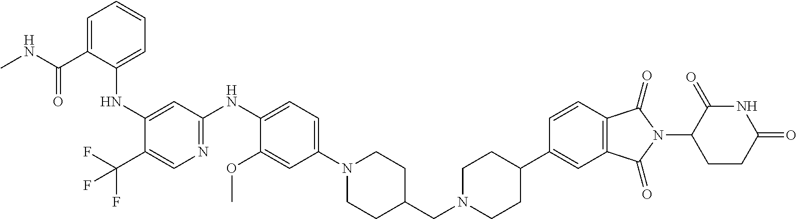 [SMILES: CNC(=O)c1ccccc1Nc1cc(Nc2ccc(N3CCC(CN4CCC(c5ccc6c(c5)C(=O)N(C5CCC(=O)NC5=O)C6=O)CC4)CC3)cc2OC)ncc1C(F)(F)F] | 2-((2-((4-(4-((4-(2-(2,6-dioxopiperidin-3-yl)- 1,3-dioxoisoindolin-5-yl)piperidin-1- yl)methyl)piperidin-1-yl)-2- methoxyphenyl)amino)-5- (trifluoromethyl)pyridin-4-yl)amino)-N- methylbenzamide |
| | 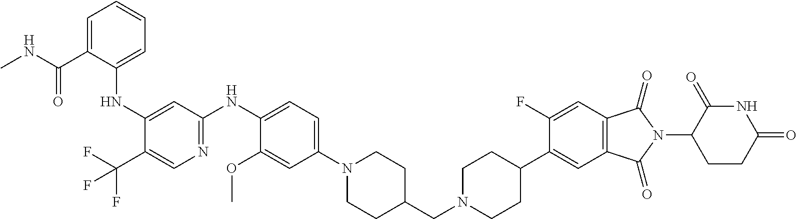 [SMILES: CNC(=O)c1ccccc1Nc1cc(Nc2ccc(N3CCC(CN4CCC(c5cc6c(cc5F)C(=O)N(C5CCC(=O)NC5=O)C6=O)CC4)CC3)cc2OC)ncc1C(F)(F)F] | 2-((2-((4-(4-((4-(2-(2,6-dioxopiperidin-3-yl)-6- fluoro-1,3-dioxoisoindolin-5-yl)piperidin-1- yl)methyl)piperidin-1-yl)-2- methoxyphenyl)amino)-5- (trifluoromethyl)pyridin-4-yl)amino)-N- methylbenzamide |
| | 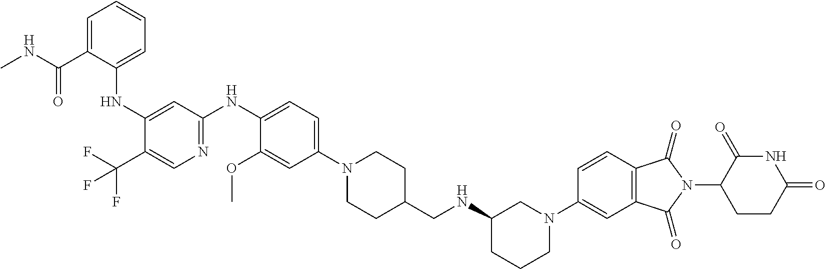 [SMILES: CNC(=O)c1ccccc1Nc1cc(Nc2ccc(N3CCC(CN[C@@H]4CCCN(c5ccc6c(c5)C(=O)N(C5CCC(=O)NC5=O)C6=O)C4)CC3)cc2OC)ncc1C(F)(F)F] | 2-((2-((4-(4-((((3R)-1-(2-(2,6-dioxopiperidin-3- yl)-1,3-dioxoisoindolin-5-yl)piperidin-3- yl)amino)methyl)piperidin-1-yl)-2- methoxyphenyl)amino)-5- (trifluoromethyl)pyridin-4-yl)amino)-N- methylbenzamide |
| | 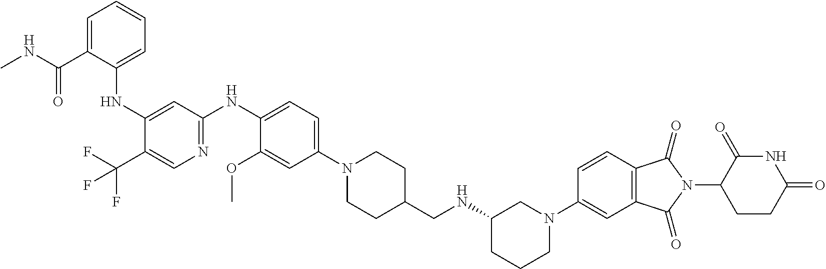 [SMILES: CNC(=O)c1ccccc1Nc1cc(Nc2ccc(N3CCC(CN[C@H]4CCCN(c5ccc6c(c5)C(=O)N(C5CCC(=O)NC5=O)C6=O)C4)CC3)cc2OC)ncc1C(F)(F)F] | 2-((2-((4-(4-((((3S)-1-(2-(2,6-dioxopiperidin-3- yl)-1,3-dioxoisoindolin-5-yl)piperidin-3- yl)amino)methyl)piperidin-1-yl)-2- methoxyphenyl)amino)-5- (trifluoromethyl)pyridin-4-yl)amino)-N- methylbenzamide |
| | 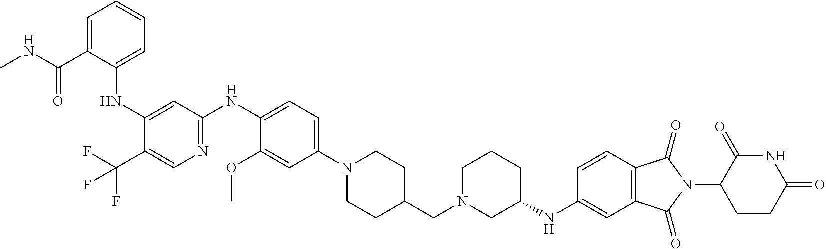 [SMILES: CNC(=O)c1ccccc1Nc1cc(Nc2ccc(N3CCC(CN4CCC[C@H](Nc5ccc6c(c5)C(=O)N(C5CCC(=O)NC5=O)C6=O)C4)CC3)cc2OC)ncc1C(F)(F)F] | 2-((2-((4-(4-(((3S)-3-((2-(2,6-dioxopiperidin-3- yl)-1,3-dioxoisoindolin-5-yl)amino)piperidin-1- yl)methyl)piperidin-1-yl)-2- methoxyphenyl)amino)-5- (trifluoromethyl)pyridin-4-yl)amino)-N- methylbenzamide |
| | 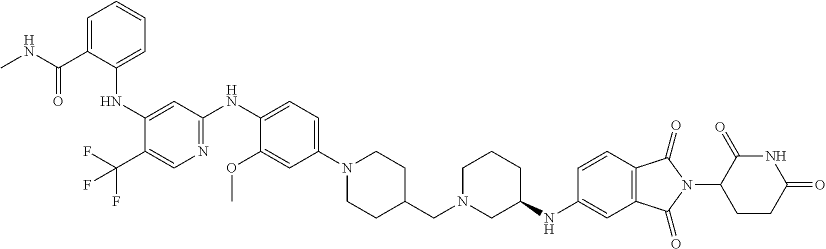 [SMILES: CNC(=O)c1ccccc1Nc1cc(Nc2ccc(N3CCC(CN4CCC[C@@H](Nc5ccc6c(c5)C(=O)N(C5CCC(=O)NC5=O)C6=O)C4)CC3)cc2OC)ncc1C(F)(F)F] | 2-((2-((4-(4-(((3R)-3-((2-(2,6-dioxopiperidin-3- yl)-1,3-dioxoisoindolin-5-yl)amino)piperidin-1- yl)methyl)piperidin-1-yl)-2- methoxyphenyl)amino)-5- (trifluoromethyl)pyridin-4-yl)amino)-N- methylbenzamide |
| | 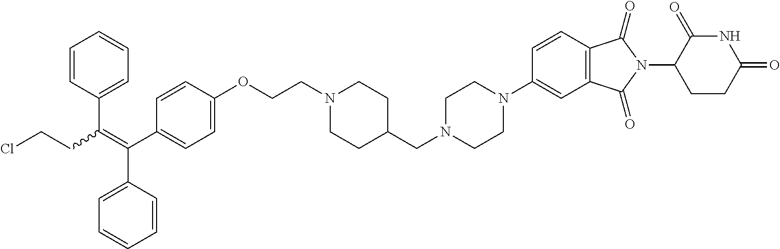 [SMILES: O=C1CCC(N2C(=O)c3ccc(N4CCN(CC5CCN(CCOc6ccc(C(=C(CCCl)c7ccccc7)c7ccccc7)cc6)CC5)CC4)cc3C2=O)C(=O)N1] | 5-(4-((1-(2-(4-(4-chloro-1,2-diphenylbut-1-en- 1-yl)phenoxy)ethyl)piperidin-4- yl)methyl)piperazin-1-yl)-2-(2,6- dioxopiperidin-3-yl)isoindoline-1,3-dione |
| | 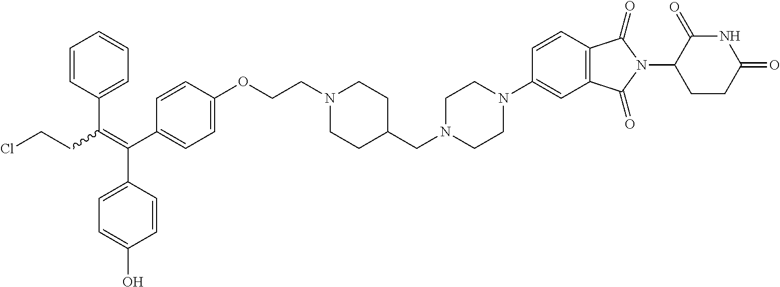 [SMILES: O=C1CCC(N2C(=O)c3ccc(N4CCN(CC5CCN(CCOc6ccc(C(=C(CCCl)c7ccccc7)c7ccc(O)cc7)cc6)CC5)CC4)cc3C2=O)C(=O)N1] | 5-(4-((1-(2-(4-(4-chloro-1-(4-hydroxyphenyl)- 2-phenylbut-1-en-1- yl)phenoxy)ethyl)piperidin-4- yl)methyl)piperazin-1-yl)-2-(2,6- dioxopiperidin-3-yl)isoindoline-1,3-dione |
| | 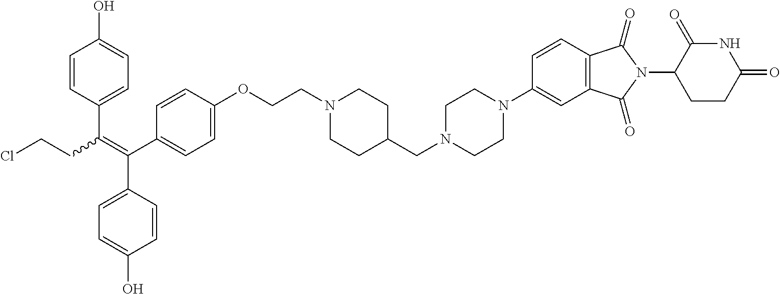 [SMILES: O=C1CCC(N2C(=O)c3ccc(N4CCN(CC5CCN(CCOc6ccc(C(=C(CCCl)c7ccc(O)cc7)c7ccc(O)cc7)cc6)CC5)CC4)cc3C2=O)C(=O)N1] | 5-(4-((1-(2-(4-(4-chloro-1,2-bis(4- hydroxyphenyl)but-1-en-1- yl)phenoxy)ethyl)piperidin-4- yl)methyl)piperazin-1-yl)-2-(2,6- dioxopiperidin-3-yl)isoindoline-1,3-dione |
| | 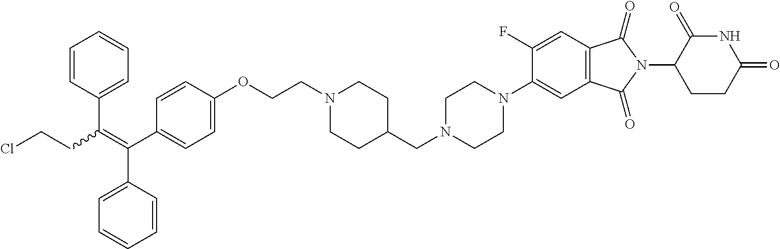 [SMILES: O=C1CCC(N2C(=O)c3cc(F)c(N4CCN(CC5CCN(CCOc6ccc(C(=C(CCCl)c7ccccc7)c7ccccc7)cc6)CC5)CC4)cc3C2=O)C(=O)N1] | 5-(4-((1-(2-(4-(4-chloro-1,2-diphenylbut-1-en- 1-yl)phenoxy)ethyl)piperidin-4- yl)methyl)piperazin-1-yl)-2-(2,6- dioxopiperidin-3-yl)-6-fluoroisoindoline-1,3- dione |
| | 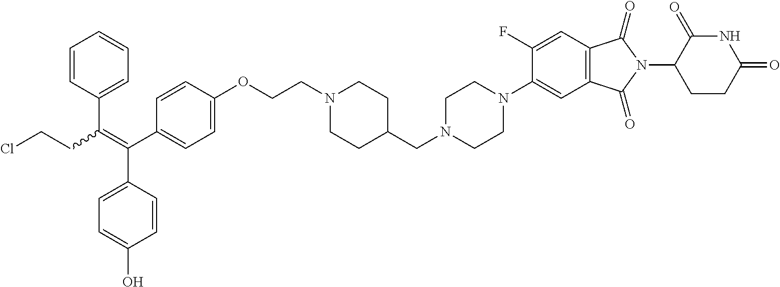 [SMILES: O=C1CCC(N2C(=O)c3cc(F)c(N4CCN(CC5CCN(CCOc6ccc(C(=C(CCCl)c7ccccc7)c7ccc(O)cc7)cc6)CC5)CC4)cc3C2=O)C(=O)N1] | 5-(4-((1-(2-(4-(4-chloro-1-(4-hydroxyphenyl)- 2-phenylbut-1-en-1- yl)phenoxy)ethyl)piperidin-4- yl)methyl)piperazin-1-yl)-2-(2,6- dioxopiperidin-3-yl)-6-fluoroisoindoline-1,3- dione |
| | 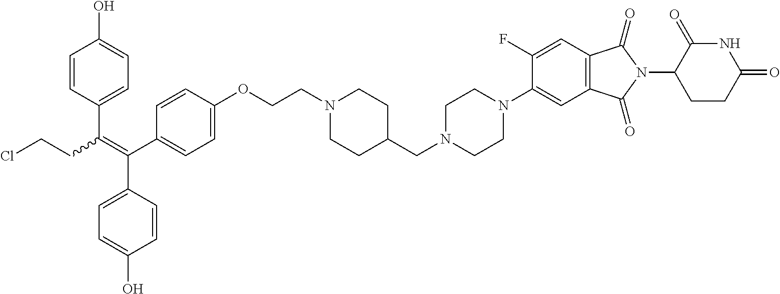 [SMILES: O=C1CCC(N2C(=O)c3cc(F)c(N4CCN(CC5CCN(CCOc6ccc(C(=C(CCCl)c7ccc(O)cc7)c7ccc(O)cc7)cc6)CC5)CC4)cc3C2=O)C(=O)N1] | 5-(4-((1-(2-(4-(4-chloro-1,2-bis(4- hydroxyphenyl)but-1-en-1- yl)phenoxy)ethyl)piperidin-4- yl)methyl)piperazin-1-yl)-2-(2,6- dioxopiperidin-3-yl)-6-fluoroisoindoline-1,3- dione |
| | 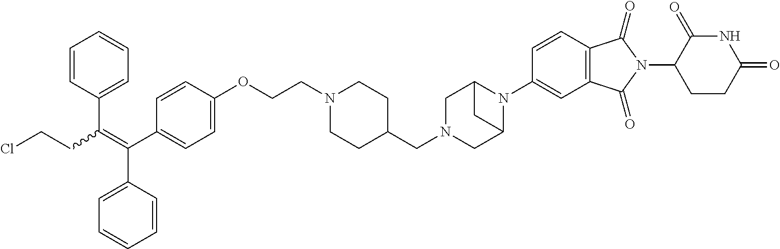 [SMILES: O=C1CCC(N2C(=O)c3ccc(N4C5CC4CN(CC4CCN(CCOc6ccc(C(=C(CCCl)c7ccccc7)c7ccccc7)cc6)CC4)C5)cc3C2=O)C(=O)N1] | 5-(3-((1-(2-(4-(4-chloro-1,2-diphenylbut-1-en- 1-yl)phenoxy)ethyl)piperidin-4-yl)methyl)-3,6- diazabicyclo[3.1.1]heptan-6-yl)-2-(2,6- dioxopiperidin-3-yl)isoindoline-1,3-dione |
| | 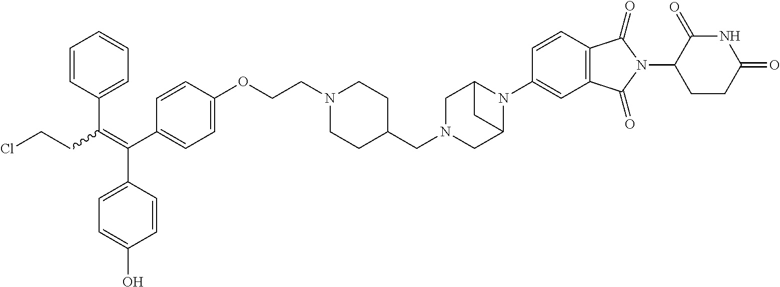 [SMILES: O=C1CCC(N2C(=O)c3ccc(N4C5CC4CN(CC4CCN(CCOc6ccc(C(=C(CCCl)c7ccccc7)c7ccc(O)cc7)cc6)CC4)C5)cc3C2=O)C(=O)N1] | 5-(3-((1-(2-(4-(4-chloro-1-(4-hydroxyphenyl)- 2-phenylbut-1-en-1- yl)phenoxy)ethyl)piperidin-4-yl)methyl)-3,6- diazabicyclo[3.1.1]heptan-6-yl)-2-(2,6- dioxopiperidin-3-yl)isoindoline-1,3-dione |
| | 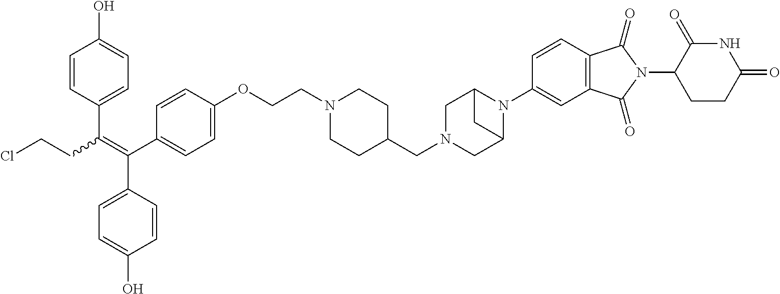 [SMILES: O=C1CCC(N2C(=O)c3ccc(N4C5CC4CN(CC4CCN(CCOc6ccc(C(=C(CCCl)c7ccc(O)cc7)c7ccc(O)cc7)cc6)CC4)C5)cc3C2=O)C(=O)N1] | 5-(3-((1-(2-(4-(4-chloro-1,2-bis(4- hydroxyphenyl)but-1-en-1- yl)phenoxy)ethyl)piperidin-4-yl)methyl)-3,6- diazabicyclo[3.1.1]heptan-6-yl)-2-(2,6- dioxopiperidin-3-yl)isoindoline-1,3-dione |
| | 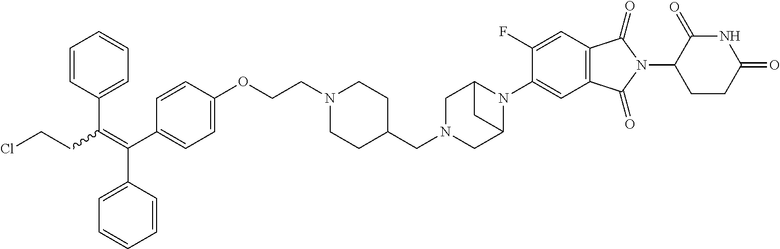 [SMILES: O=C1CCC(N2C(=O)c3cc(F)c(N4C5CC4CN(CC4CCN(CCOc6ccc(C(=C(CCCl)c7ccccc7)c7ccccc7)cc6)CC4)C5)cc3C2=O)C(=O)N1] | 5-(3-((1-(2-(4-(4-chloro-1,2-diphenylbut-1-en- 1-yl)phenoxy)ethyl)piperidin-4-yl)methyl)-3,6- diazabicyclo[3.1.1]heptan-6-yl)-2-(2,6- dioxopiperidin-3-yl)-6-fluoroisoindoline-1,3- dione |
| | 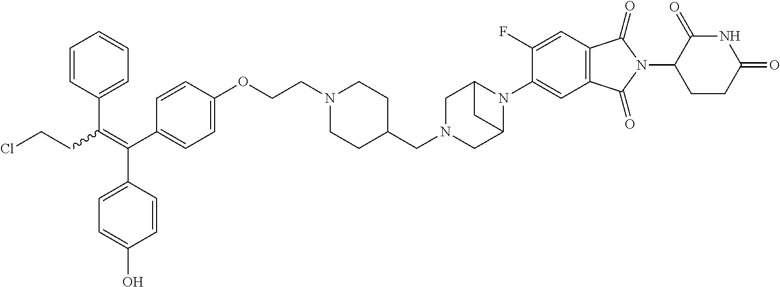 [SMILES: O=C1CCC(N2C(=O)c3cc(F)c(N4C5CC4CN(CC4CCN(CCOc6ccc(C(=C(CCCl)c7ccccc7)c7ccc(O)cc7)cc6)CC4)C5)cc3C2=O)C(=O)N1] | 5-(3-((1-(2-(4-(4-chloro-1-(4-hydroxyphenyl)- 2-phenylbut-1-en-1- yl)phenoxy)ethyl)piperidin-4-yl)methyl)-3,6- diazabicyclo[3.1.1]heptan-6-yl)-2-(2,6- dioxopiperidin-3-yl)-6-fluoroisoindoline-1,3- dione |
| | 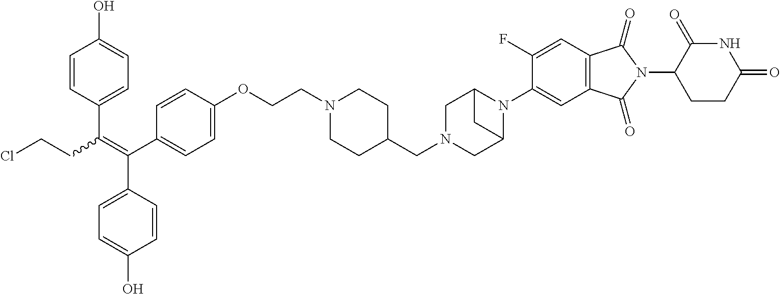 [SMILES: O=C1CCC(N2C(=O)c3cc(F)c(N4C5CC4CN(CC4CCN(CCOc6ccc(C(=C(CCCl)c7ccc(O)cc7)c7ccc(O)cc7)cc6)CC4)C5)cc3C2=O)C(=O)N1] | 5-(3-((1-(2-(4-(4-chloro-1,2-bis(4- hydroxyphenyl)but-1 -en-1- yl)phenoxy)ethyl)piperidin-4-yl)methyl)-3,6- diazabicyclo[3.1.1]heptan-6-yl)-2-(2,6- dioxopiperidin-3-yl)-6-fluoroisoindoline-1,3- dione |
| | 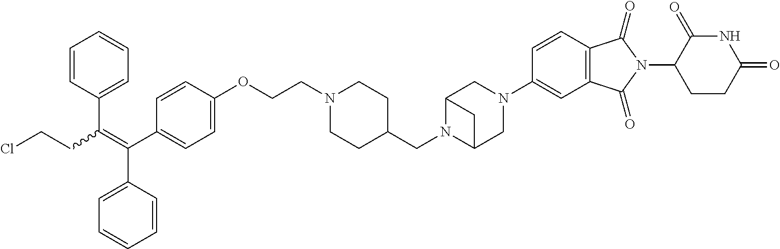 [SMILES: O=C1CCC(N2C(=O)c3ccc(N4CC5CC(C4)N5CC4CCN(CCOc5ccc(C(=C(CCCl)c6ccccc6)c6ccccc6)cc5)CC4)cc3C2=O)C(=O)N1] | 5-(6-((1-(2-(4-(4-chloro-1,2-diphenylbut-1-en- 1-yl)phenoxy)ethyl)piperidin-4-yl)methyl)-3,6- diazabicyclo[3.1.1]heptan-3-yl)-2-(2,6- dioxopiperidin-3-yl)isoindoline-1,3-dione |
| | 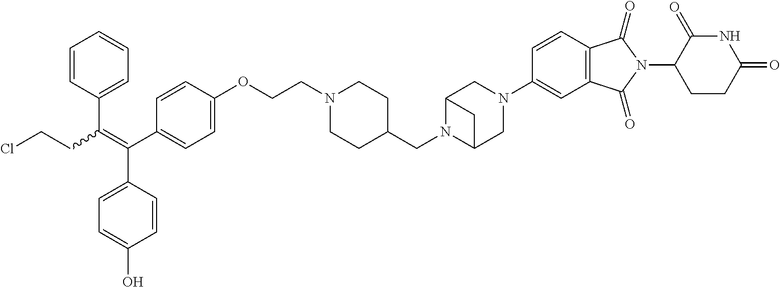 [SMILES: O=C1CCC(N2C(=O)c3ccc(N4CC5CC(C4)N5CC4CCN(CCOc5ccc(C(=C(CCCl)c6ccccc6)c6ccc(O)cc6)cc5)CC4)cc3C2=O)C(=O)N1] | 5-(6-((1-(2-(4-(4-chloro-1-(4-hydroxyphenyl)- 2-phenylbut-1 -en-1- yl)phenoxy)ethyl)piperidin-4-yl)methyl)-3,6- diazabicyclo[3.1.1]heptan-3-yl)-2-(2,6- dioxopiperidin-3-yl)isoindoline-1,3-dione |
| | 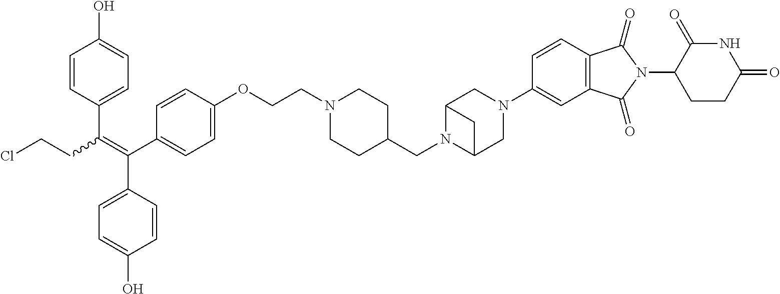 [SMILES: O=C1CCC(N2C(=O)c3ccc(N4CC5CC(C4)N5CC4CCN(CCOc5ccc(C(=C(CCCl)c6ccc(O)cc6)c6ccc(O)cc6)cc5)CC4)cc3C2=O)C(=O)N1] | 5-(6-((1-(2-(4-(4-chloro-1,2-bis(4- hydroxyphenyl)but-1-en-1- yl)phenoxy)ethyl)piperidin-4-yl)methyl)-3,6- diazabicyclo[3.1.1]heptan-3-yl)-2-(2,6- dioxopiperidin-3-yl)isoindoline-1,3-dione |
| | 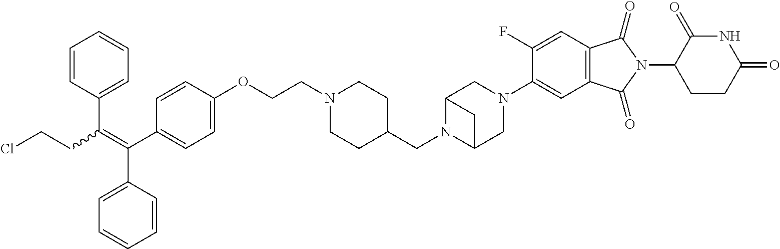 [SMILES: O=C1CCC(N2C(=O)c3cc(F)c(N4CC5CC(C4)N5CC4CCN(CCOc5ccc(C(=C(CCCl)c6ccccc6)c6ccccc6)cc5)CC4)cc3C2=O)C(=O)N1] | 5-(6-((1-(2-(4-(4-chloro-1,2-diphenylbut-1-en- 1-yl)phenoxy)ethyl)piperidin-4-yl)methyl)-3,6- diazabicyclo[3.1.1]heptan-3-yl)-2-(2,6- dioxopiperidin-3-yl)-6-fluoroisoindoline-1,3- dione |
| | 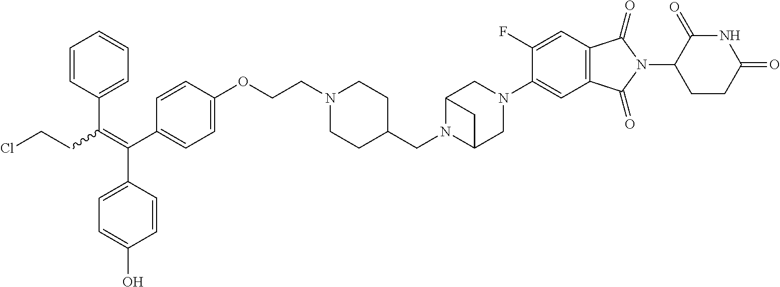 [SMILES: O=C1CCC(N2C(=O)c3cc(F)c(N4CC5CC(C4)N5CC4CCN(CCOc5ccc(C(=C(CCCl)c6ccccc6)c6ccc(O)cc6)cc5)CC4)cc3C2=O)C(=O)N1] | 5-(6-((1-(2-(4-(4-chloro-1-(4-hydroxyphenyl)- 2-phenylbut-1-en-1- yl)phenoxy)ethyl)piperidin-4-yl)methyl)-3,6- diazabicyclo[3.1.1]heptan-3-yl)-2-(2,6- dioxopiperidin-3-yl)-6-fluoroisoindoline-1,3- dione |
| | 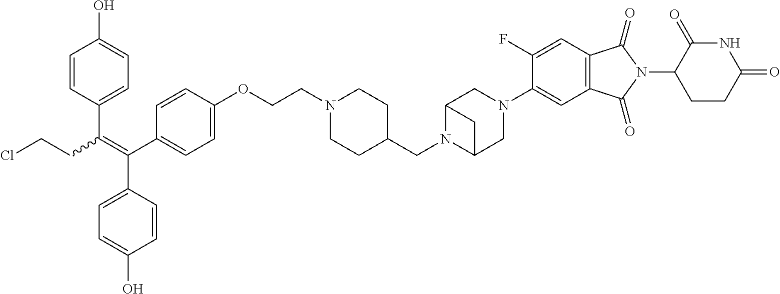 [SMILES: O=C1CCC(N2C(=O)c3cc(F)c(N4CC5CC(C4)N5CC4CCN(CCOc5ccc(C(=C(CCCl)c6ccc(O)cc6)c6ccc(O)cc6)cc5)CC4)cc3C2=O)C(=O)N1] | 5-(6-((1-(2-(4-(4-chloro-1,2-bis(4- hydroxyphenyl)but-1-en-1- yl)phenoxy)ethyl)piperidin-4-yl)methyl)-3,6- diazabicyclo[3.1.1]heptan-3-yl)-2-(2,6- dioxopiperidin-3-yl)-6-fluoroisoindoline-1,3- dione |
| | 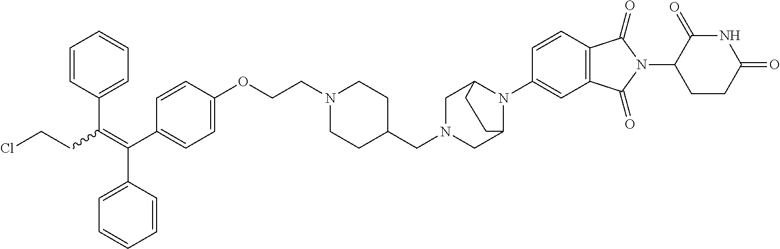 [SMILES: O=C1CCC(N2C(=O)c3ccc(N4C5CCC4CN(CC4CCN(CCOc6ccc(C(=C(CCCl)c7ccccc7)c7ccccc7)cc6)CC4)C5)cc3C2=O)C(=O)N1] | 5-(3-((1-(2-(4-(4-chloro-1,2-diphenylbut-1-en- 1-yl)phenoxy)ethyl)piperidin-4-yl)methyl)-3,8- diazabicyclo[3.2.1]octan-8-yl)-2-(2,6- dioxopiperidin-3-yl)isoindoline-1,3-dione |
| | 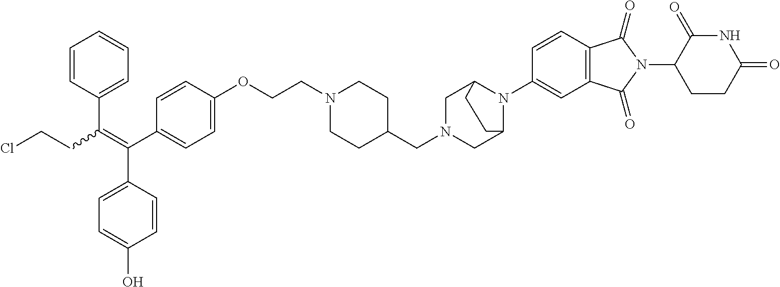 [SMILES: O=C1CCC(N2C(=O)c3ccc(N4C5CCC4CN(CC4CCN(CCOc6ccc(C(=C(CCCl)c7ccccc7)c7ccc(O)cc7)cc6)CC4)C5)cc3C2=O)C(=O)N1] | 5-(3-((1-(2-(4-(4-chloro-1-(4-hydroxyphenyl)- 2-phenylbut-1-en-1- yl)phenoxy)ethyl)piperidin-4-yl)methyl)-3,8- diazabicyclo[3.2.1]octan-8-yl)-2-(2,6- dioxopiperidin-3-yl)isoindoline-1,3-dione |
| | 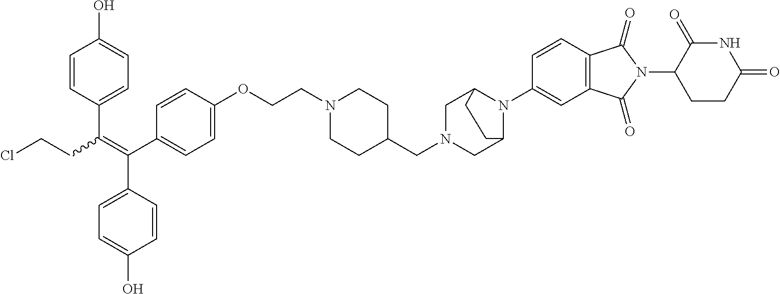 [SMILES: O=C1CCC(N2C(=O)c3ccc(N4C5CCC4CN(CC4CCN(CCOc6ccc(C(=C(CCCl)c7ccc(O)cc7)c7ccc(O)cc7)cc6)CC4)C5)cc3C2=O)C(=O)N1] | 5-(3-((1-(2-(4-(4-chloro-1,2-bis(4- hydroxyphenyl)but-1-en-1- yl)phenoxy)ethyl)piperidin-4-yl)methyl)-3,8- diazabicyclo[3.2.1]octan-8-yl)-2-(2,6- dioxopiperidin-3-yl)isoindoline-1,3-dione |
| | 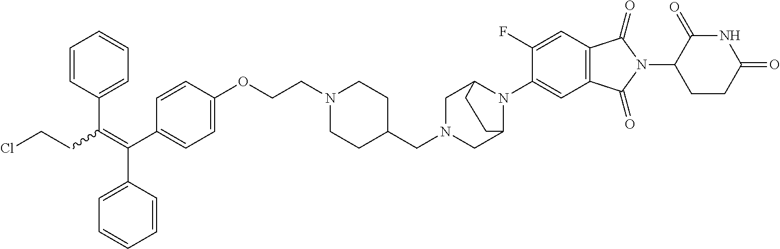 [SMILES: O=C1CCC(N2C(=O)c3cc(F)c(N4C5CCC4CN(CC4CCN(CCOc6ccc(C(=C(CCCl)c7ccccc7)c7ccccc7)cc6)CC4)C5)cc3C2=O)C(=O)N1] | 5-(3-((1-(2-(4-(4-chloro-1,2-diphenylbut-1-en- 1-yl)phenoxy)ethyl)piperidin-4-yl)methyl)-3,8- diazabicyclo[3.2.1]octan-8-yl)-2-(2,6- dioxopiperidin-3-yl)-6-fluoroisoindoline-1,3- dione |
| | 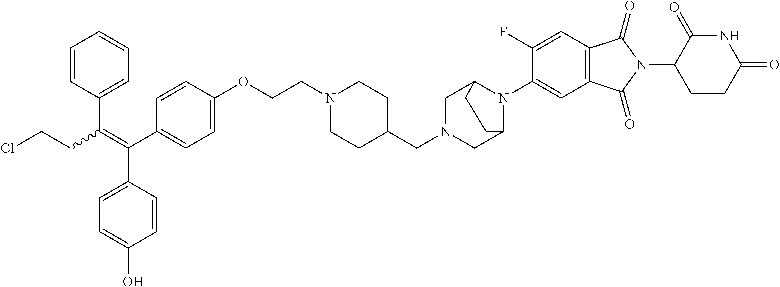 [SMILES: O=C1CCC(N2C(=O)c3cc(F)c(N4C5CCC4CN(CC4CCN(CCOc6ccc(C(=C(CCCl)c7ccccc7)c7ccc(O)cc7)cc6)CC4)C5)cc3C2=O)C(=O)N1] | 5-(3-((1-(2-(4-(4-chloro-1-(4-hydroxyphenyl)- 2-phenylbut-1-en-1- yl)phenoxy)ethyl)piperidin-4-yl)methyl)-3,8- diazabicyclo[3.2.1]octan-8-yl)-2-(2,6- dioxopiperidin-3-yl)-6-fluoroisoindoline-1,3- dione |
| | 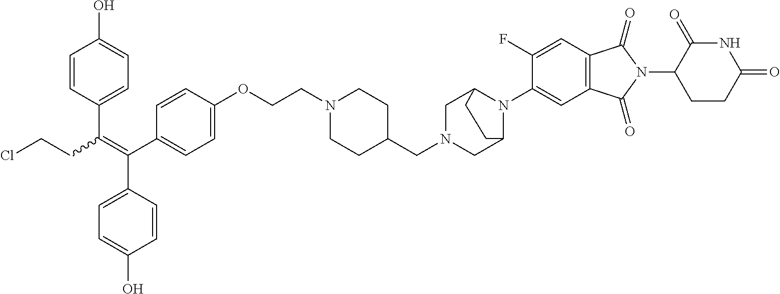 [SMILES: O=C1CCC(N2C(=O)c3cc(F)c(N4C5CCC4CN(CC4CCN(CCOc6ccc(C(=C(CCCl)c7ccc(O)cc7)c7ccc(O)cc7)cc6)CC4)C5)cc3C2=O)C(=O)N1] | 5-(3-((1-(2-(4-(4-chloro-1,2-bis(4- hydroxyphenyl)but-1-en-1- yl)phenoxy)ethyl)piperidin-4-yl)methyl)-3,8- diazabicyclo[3.2.1]octan-8-yl)-2-(2,6- dioxopiperidin-3-yl)-6-fluoroisoindoline-1,3- dione |
| | 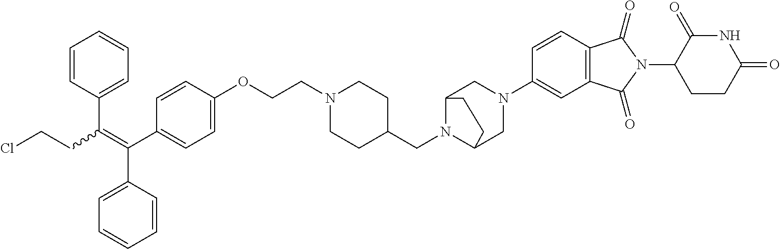 [SMILES: O=C1CCC(N2C(=O)c3ccc(N4CC5CCC(C4)N5CC4CCN(CCOc5ccc(C(=C(CCCl)c6ccccc6)c6ccccc6)cc5)CC4)cc3C2=O)C(=O)N1] | 5-(8-((1-(2-(4-(4-chloro-1,2-diphenylbut-1-en- 1-yl)phenoxy)ethyl)piperidin-4-yl)methyl)-3,8- diazabicyclo[3.2.1]octan-3-yl)-2-(2,6- dioxopiperidin-3-yl)isoindoline-1,3-dione |
| | 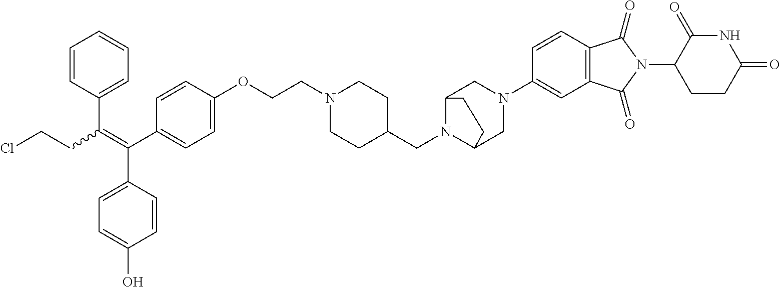 [SMILES: O=C1CCC(N2C(=O)c3ccc(N4CC5CCC(C4)N5CC4CCN(CCOc5ccc(C(=C(CCCl)c6ccccc6)c6ccc(O)cc6)cc5)CC4)cc3C2=O)C(=O)N1] | 5-(8-((1-(2-(4-(4-chloro-1-(4-hydroxyphenyl)- 2-phenylbut-1-en-1- yl)phenoxy)ethyl)piperidin-4-yl)methyl)-3,8- diazabicyclo[3.2.1]octan-3-yl)-2-(2,6- dioxopiperidin-3-yl)isoindoline-1,3-dione |
| | 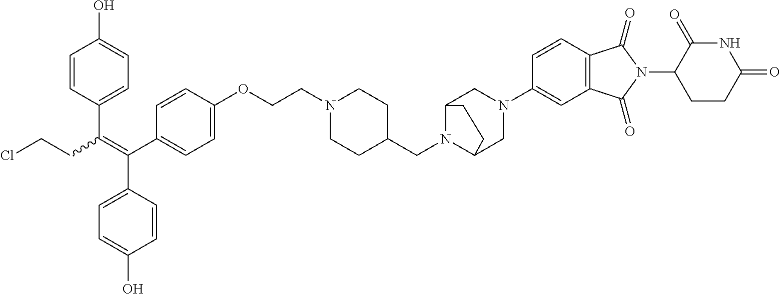 [SMILES: O=C1CCC(N2C(=O)c3ccc(N4CC5CCC(C4)N5CC4CCN(CCOc5ccc(C(=C(CCCl)c6ccc(O)cc6)c6ccc(O)cc6)cc5)CC4)cc3C2=O)C(=O)N1] | 5-(8-((1-(2-(4-(4-chloro-1,2-bis(4- hydroxyphenyl)but-1-en-1- yl)phenoxy)ethyl)piperidin-4-yl)methyl)-3,8- diazabicyclo[3.2.1]octan-3-yl)-2-(2,6- dioxopiperidin-3-yl)isoindoline-1,3-dione |
| | 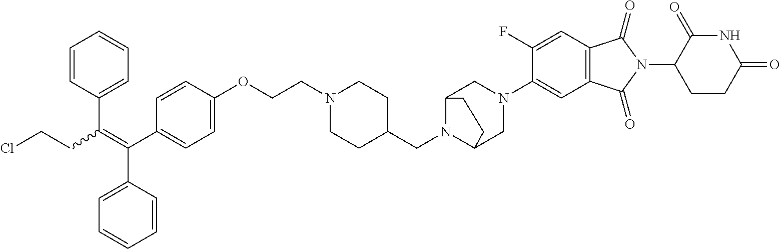 [SMILES: O=C1CCC(N2C(=O)c3cc(F)c(N4CC5CCC(C4)N5CC4CCN(CCOc5ccc(C(=C(CCCl)c6ccccc6)c6ccccc6)cc5)CC4)cc3C2=O)C(=O)N1] | 5-(8-((1-(2-(4-(4-chloro-1,2-diphenylbut-1-en- 1-yl)phenoxy)ethyl)piperidin-4-yl)methyl)-3,8- diazabicyclo[3.2.1]octan-3-yl)-2-(2,6- dioxopiperidin-3-yl)-6-fluoroisoindoline-1,3- dione |
| | 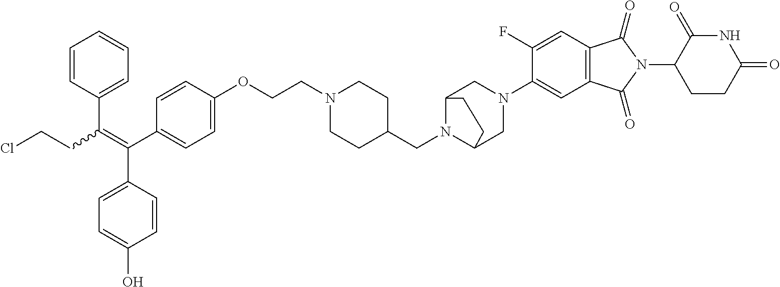 [SMILES: O=C1CCC(N2C(=O)c3cc(F)c(N4CC5CCC(C4)N5CC4CCN(CCOc5ccc(C(=C(CCCl)c6ccccc6)c6ccc(O)cc6)cc5)CC4)cc3C2=O)C(=O)N1] | 5-(8-((1-(2-(4-(4-chloro-1-(4-hydroxyphenyl)- 2-phenylbut-1-en-1- yl)phenoxy)ethyl)piperidin-4-yl)methyl)-3,8- diazabicyclo[3.2.1]octan-3-yl)-2-(2,6- dioxopiperidin-3-yl)-6-fluoroisoindoline-1,3- dione |
| | 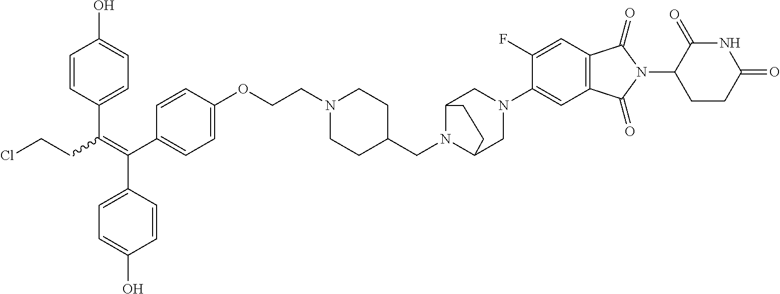 [SMILES: O=C1CCC(N2C(=O)c3cc(F)c(N4CC5CCC(C4)N5CC4CCN(CCOc5ccc(C(=C(CCCl)c6ccc(O)cc6)c6ccc(O)cc6)cc5)CC4)cc3C2=O)C(=O)N1] | 5-(8-((1-(2-(4-(4-chloro-1,2-bis(4- hydroxyphenyl)but-1-en-1- yl)phenoxy)ethyl)piperidin-4-yl)methyl)-3,8- diazabicyclo[3.2.1]octan-3-yl)-2-(2,6- dioxopiperidin-3-yl)-6-fluoroisoindoline-1,3- dione |
| | 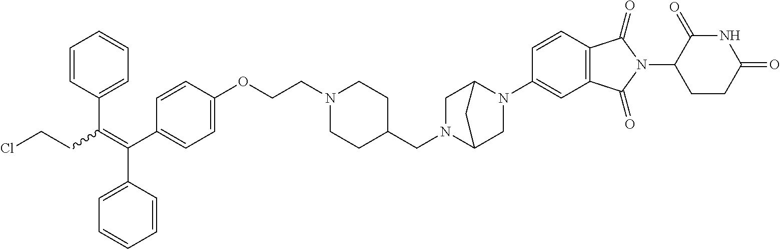 [SMILES: O=C1CCC(N2C(=O)c3ccc(N4CC5CC4CN5CC4CCN(CCOc5ccc(C(=C(CCCl)c6ccccc6)c6ccccc6)cc5)CC4)cc3C2=O)C(=O)N1] | 5-(5-((1-(2-(4-(4-chloro-1,2-diphenylbut-1-en- 1-yl)phenoxy)ethyl)piperidin-4-yl)methyl)-2,5- diazabicyclo[2.2.1]heptan-2-yl)-2-(2,6- dioxopiperidin-3-yl)isoindoline-1,3-dione |
| | 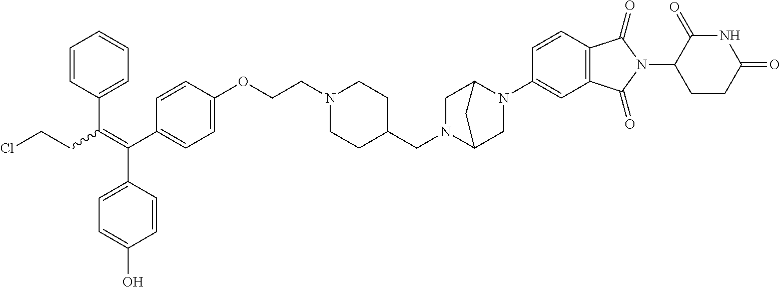 [SMILES: O=C1CCC(N2C(=O)c3ccc(N4CC5CC4CN5CC4CCN(CCOc5ccc(C(=C(CCCl)c6ccccc6)c6ccc(O)cc6)cc5)CC4)cc3C2=O)C(=O)N1] | 5-(5-((1-(2-(4-(4-chloro-1-(4-hydroxyphenyl)- 2-phenylbut-1-en-1- yl)phenoxy)ethyl)piperidin-4-yl)methyl)-2,5- diazabicyclo[2.2.1]heptan-2-yl)-2-(2,6- dioxopiperidin-3-yl)isoindoline-1,3-dione |
| | 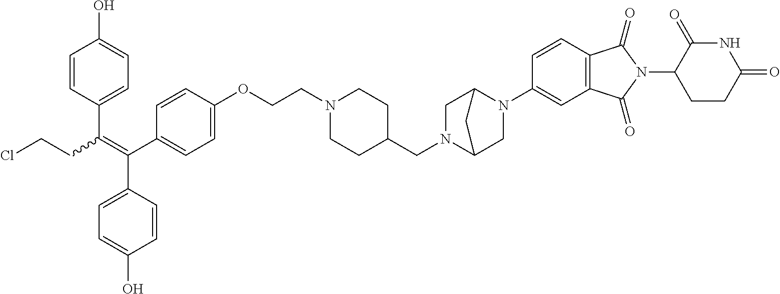 [SMILES: O=C1CCC(N2C(=O)c3ccc(N4CC5CC4CN5CC4CCN(CCOc5ccc(C(=C(CCCl)c6ccc(O)cc6)c6ccc(O)cc6)cc5)CC4)cc3C2=O)C(=O)N1] | 5-(5-((1-(2-(4-(4-chloro-1,2-bis(4- hydroxyphenyl)but-1-en-1- yl)phenoxy)ethyl)piperidin-4-yl)methyl)-2,5- diazabicyclo[2.2.1]heptan-2-yl)-2-(2,6- dioxopiperidin-3-yl)isoindoline-1,3-dione |
| | 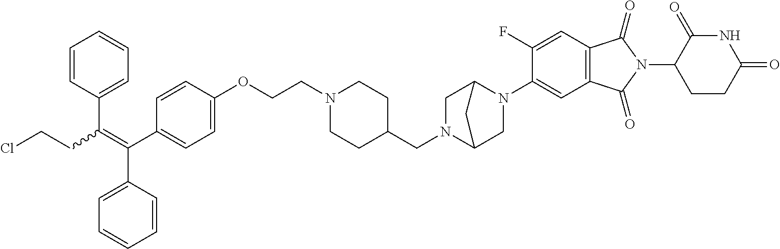 [SMILES: O=C1CCC(N2C(=O)c3cc(F)c(N4CC5CC4CN5CC4CCN(CCOc5ccc(C(=C(CCCl)c6ccccc6)c6ccccc6)cc5)CC4)cc3C2=O)C(=O)N1] | 5-(5-((1-(2-(4-(4-chloro-1,2-diphenylbut-1-en- 1-yl)phenoxy)ethyl)piperidin-4-yl)methyl)-2,5- diazabicyclo[2.2.1]heptan-2-yl)-2-(2,6- dioxopiperidin-3-yl)-6-fluoroisoindoline-1,3- dione |
| | 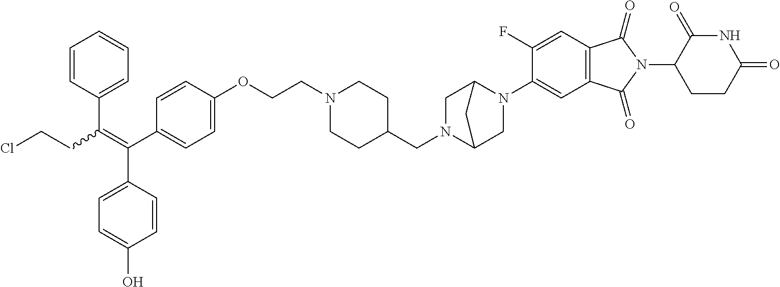 [SMILES: O=C1CCC(N2C(=O)c3cc(F)c(N4CC5CC4CN5CC4CCN(CCOc5ccc(C(=C(CCCl)c6ccccc6)c6ccc(O)cc6)cc5)CC4)cc3C2=O)C(=O)N1] | 5-(5-((1-(2-(4-(4-chloro-1-(4-hydroxyphenyl)- 2-phenylbut-1-en-1- yl)phenoxy)ethyl)piperidin-4-yl)methyl)-2,5- diazabicyclo[2.2.1]heptan-2-yl)-2-(2,6- dioxopiperidin-3-yl)-6-fluoroisoindoline-1,3- dione |
| | 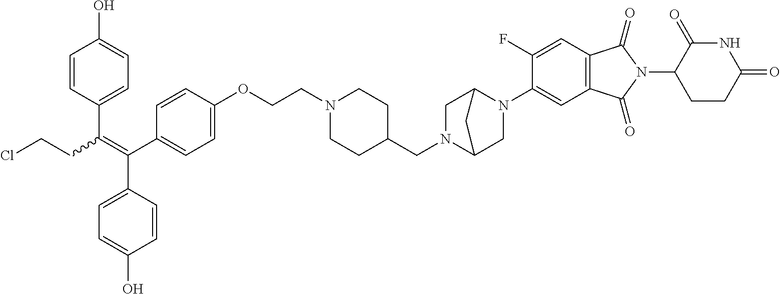 [SMILES: O=C1CCC(N2C(=O)c3cc(F)c(N4CC5CC4CN5CC4CCN(CCOc5ccc(C(=C(CCCl)c6ccc(O)cc6)c6ccc(O)cc6)cc5)CC4)cc3C2=O)C(=O)N1] | 5-(5-((1-(2-(4-(4-chloro-1,2-bis(4- hydroxyphenyl)but-1-en-1- yl)phenoxy)ethyl)piperidin-4-yl)methyl)-2,5- diazabicyclo[2.2.1]heptan-2-yl)-2-(2,6- dioxopiperidin-3-yl)-6-fluoroisoindoline-1,3- dione |
| | 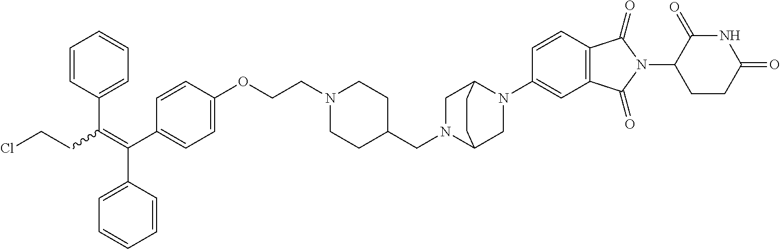 [SMILES: O=C1CCC(N2C(=O)c3ccc(N4CC5CCC4CN5CC4CCN(CCOc5ccc(C(=C(CCCl)c6ccccc6)c6ccccc6)cc5)CC4)cc3C2=O)C(=O)N1] | 5-(5-((1-(2-(4-(4-chloro-1,2-diphenylbut-1-en- 1-yl)phenoxy)ethyl)piperidin-4-yl)methyl)-2,5- diazabicyclo[2.2.2]octan-2-yl)-2-(2,6- dioxopiperidin-3-yl)isoindoline-1,3-dione |
| | 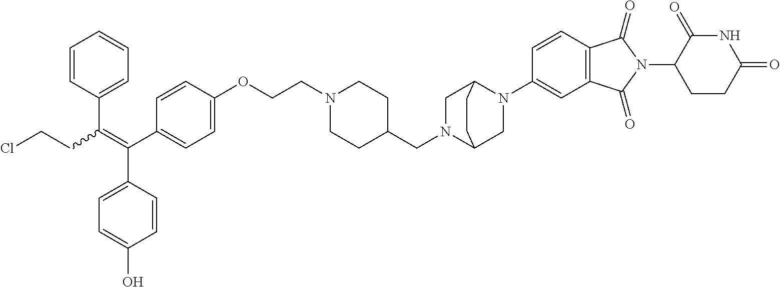 [SMILES: O=C1CCC(N2C(=O)c3ccc(N4CC5CCC4CN5CC4CCN(CCOc5ccc(C(=C(CCCl)c6ccccc6)c6ccc(O)cc6)cc5)CC4)cc3C2=O)C(=O)N1] | 5-(5-((1-(2-(4-(4-chloro-1-(4-hydroxyphenyl)- 2-phenylbut-1-en-1- yl)phenoxy)ethyl)piperidin-4-yl)methyl)-2,5- diazabicyclo[2.2.2]octan-2-yl)-2-(2,6- dioxopiperidin-3-yl)isoindoline-1,3-dione |
| | 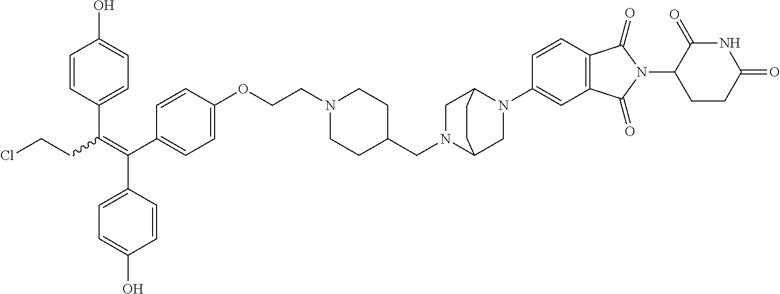 [SMILES: O=C1CCC(N2C(=O)c3ccc(N4CC5CCC4CN5CC4CCN(CCOc5ccc(C(=C(CCCl)c6ccc(O)cc6)c6ccc(O)cc6)cc5)CC4)cc3C2=O)C(=O)N1] | 5-(5-((1-(2-(4-(4-chloro-1,2-bis(4- hydroxyphenyl)but-1-en-1- yl)phenoxy)ethyl)piperidin-4-yl)methyl)-2,5- diazabicyclo[2.2.2]octan-2-yl)-2-(2,6- dioxopiperidin-3-yl)isoindoline-1,3-dione |
| | 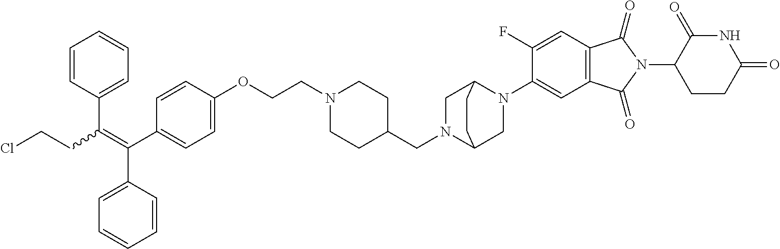 [SMILES: O=C1CCC(N2C(=O)c3cc(F)c(N4CC5CCC4CN5CC4CCN(CCOc5ccc(C(=C(CCCl)c6ccccc6)c6ccccc6)cc5)CC4)cc3C2=O)C(=O)N1] | 5-(5-((1-(2-(4-(4-chloro-1,2-diphenylbut-1-en- 1-yl)phenoxy)ethyl)piperidin-4-yl)methyl)-2,5- diazabicyclo[2.2.2]octan-2-yl)-2-(2,6- dioxopiperidin-3-yl)-6-fluoroisoindoline-1,3- dione |
| | 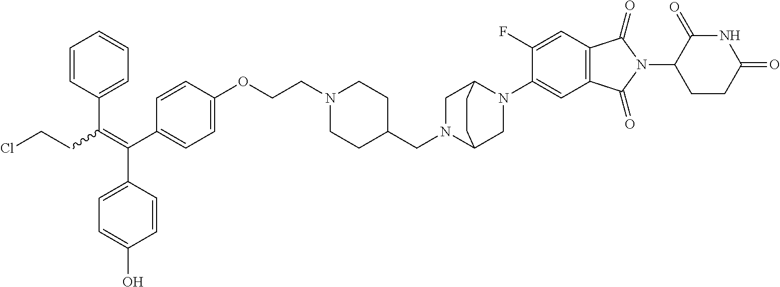 [SMILES: O=C1CCC(N2C(=O)c3cc(F)c(N4CC5CCC4CN5CC4CCN(CCOc5ccc(C(=C(CCCl)c6ccccc6)c6ccc(O)cc6)cc5)CC4)cc3C2=O)C(=O)N1] | 5-(5-((1-(2-(4-(4-chloro-1-(4-hydroxyphenyl)- 2-phenylbut-1-en-1- yl)phenoxy)ethyl)piperidin-4-yl)methyl)-2,5- diazabicyclo[2.2.2]octan-2-yl)-2-(2,6- dioxopiperidin-3-yl)-6-fluoroisoindoline-1,3- dione |
| | 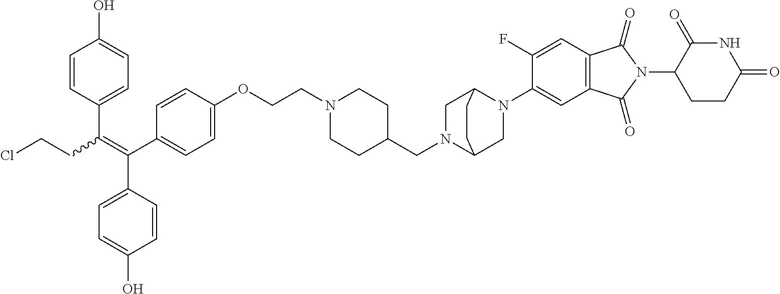 [SMILES: O=C1CCC(N2C(=O)c3cc(F)c(N4CC5CCC4CN5CC4CCN(CCOc5ccc(C(=C(CCCl)c6ccc(O)cc6)c6ccc(O)cc6)cc5)CC4)cc3C2=O)C(=O)N1] | 5-(5-((1-(2-(4-(4-chloro-1,2-bis(4- hydroxyphenyl)but-1-en-1- yl)phenoxy)ethyl)piperidin-4-yl)methyl)-2,5- diazabicyclo[2.2.2]octan-2-yl)-2-(2,6- dioxopiperidin-3-yl)-6-fluoroisoindoline-1,3- dione |
| | 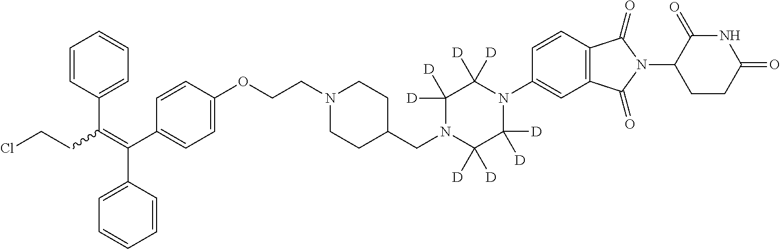 [SMILES: [2H]C1([2H])N(CC2CCN(CCOc3ccc(C(=C(CCCl)c4ccccc4)c4ccccc4)cc3)CC2)C([2H])([2H])C([2H])([2H])N(c2ccc3c(c2)C(=O)N(C2CCC(=O)NC2=O)C3=O)C1([2H])[2H]] | 5-(4-((1-(2-(4-(4-chloro-1,2-diphenylbut-1-en- 1-yl)phenoxy)ethyl)piperidin-4- yl)methyl)piperazin-1-yl-2,2,3,3,5,5,6,6-d₈)-2- (2,6-dioxopiperidin-3-yl)isoindoline-1,3-dione |
| | 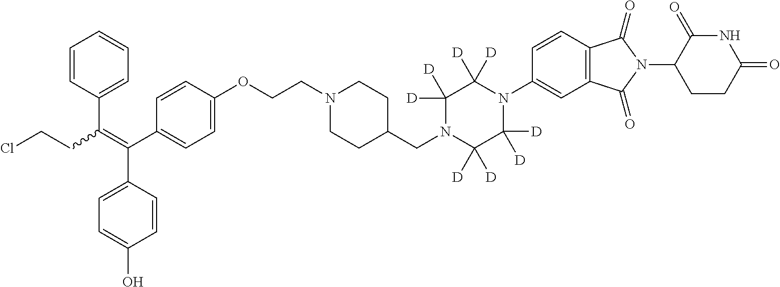 [SMILES: [2H]C1([2H])N(CC2CCN(CCOc3ccc(C(=C(CCCl)c4ccccc4)c4ccc(O)cc4)cc3)CC2)C([2H])([2H])C([2H])([2H])N(c2ccc3c(c2)C(=O)N(C2CCC(=O)NC2=O)C3=O)C1([2H])[2H]] | 5-(4-((1-(2-(4-(4-chloro-1-(4-hydroxyphenyl)- 2-phenylbut-1 -en-1- yl)phenoxy)ethyl)piperidin-4- yl)methyl)piperazin-1-yl-2,2,3,3,5,5,6,6-d₈)-2- (2,6-dioxopiperidin-3-yl)isoindoline-1,3-dione |
| | 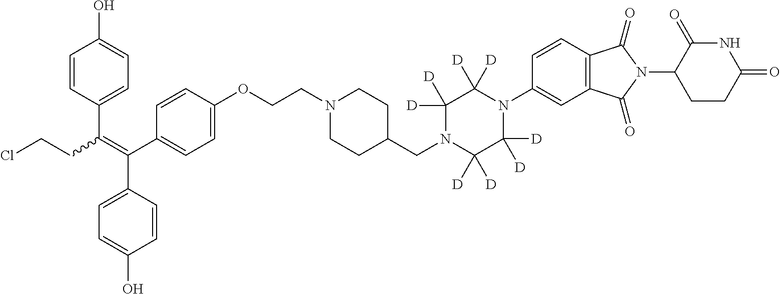 [SMILES: [2H]C1([2H])N(CC2CCN(CCOc3ccc(C(=C(CCCl)c4ccc(O)cc4)c4ccc(O)cc4)cc3)CC2)C([2H])([2H])C([2H])([2H])N(c2ccc3c(c2)C(=O)N(C2CCC(=O)NC2=O)C3=O)C1([2H])[2H]] | 5-(4-((1-(2-(4-(4-chloro-1,2-bis(4- hydroxyphenyl)but-1-en-1- yl)phenoxy)ethyl)piperidin-4- yl)methyl)piperazin-1-yl-2,2,3,3,5,5,6,6-d₈)-2- (2,6-dioxopiperidin-3-yl)isoindoline-1,3-dione |
| | 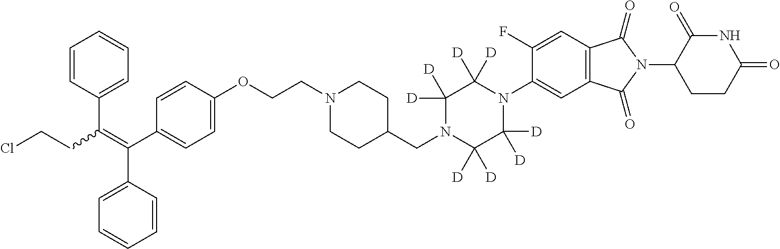 [SMILES: [2H]C1([2H])N(CC2CCN(CCOc3ccc(C(=C(CCCl)c4ccccc4)c4ccccc4)cc3)CC2)C([2H])([2H])C([2H])([2H])N(c2cc3c(cc2F)C(=O)N(C2CCC(=O)NC2=O)C3=O)C1([2H])[2H]] | 5-(4-((1-(2-(4-(4-chloro-1,2-diphenylbut-1-en- 1-yl)phenoxy)ethyl)piperidin-4- yl)methyl)piperazin-1-yl-2,2,3,3,5,5,6,6-d₈)-2- (2,6-dioxopiperidin-3-yl)-6-fluoroisoindoline- 1,3-dione |
| | 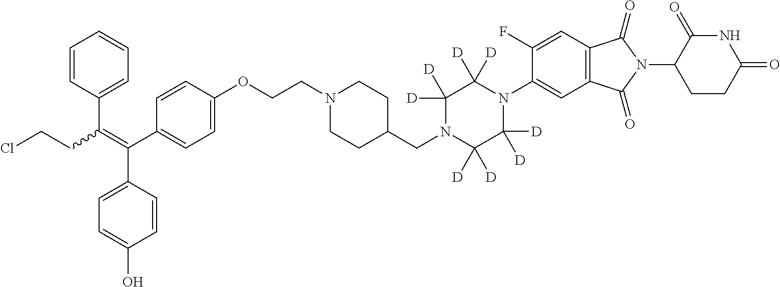 [SMILES: [2H]C1([2H])N(CC2CCN(CCOc3ccc(C(=C(CCCl)c4ccccc4)c4ccc(O)cc4)cc3)CC2)C([2H])([2H])C([2H])([2H])N(c2cc3c(cc2F)C(=O)N(C2CCC(=O)NC2=O)C3=O)C1([2H])[2H]] | 5-(4-((1-(2-(4-(4-chloro-1-(4-hydroxyphenyl)- 2-phenylbut-1-en-1- yl)phenoxy)ethyl)piperidin-4- yl)methyl)piperazin-1-yl-2,2,3,3,5,5,6,6-d₈)-2- (2,6-dioxopiperidin-3-yl)-6-fluoroisoindoline- 1,3-dione |
| | 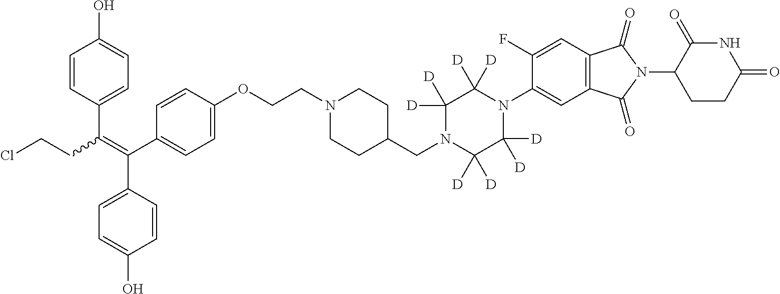 [SMILES: [2H]C1([2H])N(CC2CCN(CCOc3ccc(C(=C(CCCl)c4ccc(O)cc4)c4ccc(O)cc4)cc3)CC2)C([2H])([2H])C([2H])([2H])N(c2cc3c(cc2F)C(=O)N(C2CCC(=O)NC2=O)C3=O)C1([2H])[2H]] | 5-(4-((1-(2-(4-(4-chloro-1,2-bis(4- hydroxyphenyl)but-1-en-1- yl)phenoxy)ethyl)piperidin-4- yl)methyl)piperazin-1-yl-2,2,3,3,5,5,6,6-d₈)-2- (2,6-dioxopiperidin-3-yl)-6-fluoroisoindoline- 1,3-dione |
| | 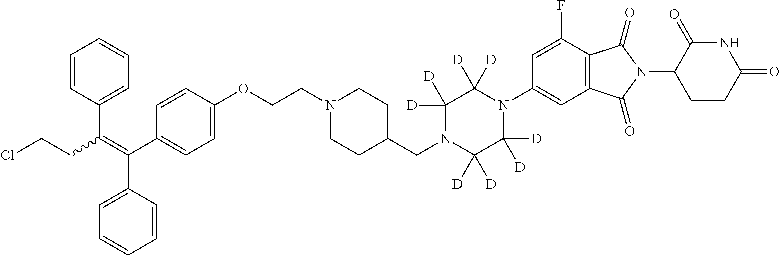 [SMILES: [2H]C1([2H])N(CC2CCN(CCOc3ccc(C(=C(CCCl)c4ccccc4)c4ccccc4)cc3)CC2)C([2H])([2H])C([2H])([2H])N(c2cc(F)c3c(c2)C(=O)N(C2CCC(=O)NC2=O)C3=O)C1([2H])[2H]] | 6-(4-((1-(2-(4-(4-chloro-1,2-diphenylbut-1-en- 1-yl)phenoxy)ethyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 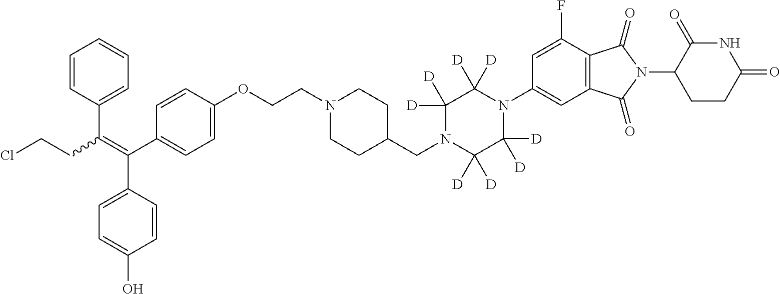 [SMILES: [2H]C1([2H])N(CC2CCN(CCOc3ccc(C(=C(CCCl)c4ccccc4)c4ccc(O)cc4)cc3)CC2)C([2H])([2H])C([2H])([2H])N(c2cc(F)c3c(c2)C(=O)N(C2CCC(=O)NC2=O)C3=O)C1([2H])[2H]] | 6-(4-((1-(2-(4-(4-chloro-1-(4-hydroxyphenyl)- 2-phenylbut-1-en-1- yl)phenoxy)ethyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 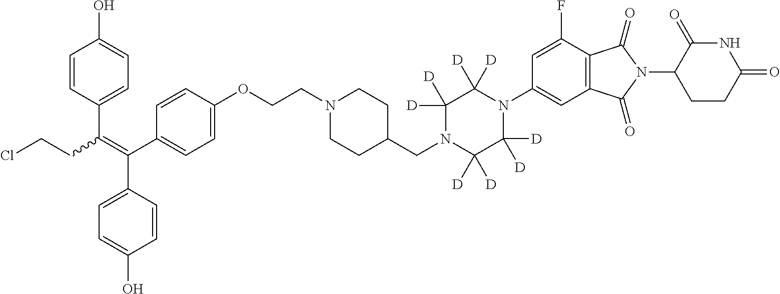 [SMILES: [2H]C1([2H])N(CC2CCN(CCOc3ccc(C(=C(CCCl)c4ccc(O)cc4)c4ccc(O)cc4)cc3)CC2)C([2H])([2H])C([2H])([2H])N(c2cc(F)c3c(c2)C(=O)N(C2CCC(=O)NC2=O)C3=O)C1([2H])[2H]] | 6-(4-((1-(2-(4-(4-chloro-1,2-bis(4- hydroxyphenyl)but-1-en-1- yl)phenoxy)ethyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 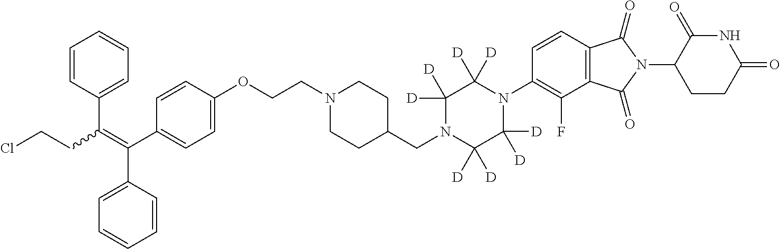 [SMILES: [2H]C1([2H])N(CC2CCN(CCOc3ccc(C(=C(CCCl)c4ccccc4)c4ccccc4)cc3)CC2)C([2H])([2H])C([2H])([2H])N(c2ccc3c(c2F)C(=O)N(C2CCC(=O)NC2=O)C3=O)C1([2H])[2H]] | 5-(4-((1-(2-(4-(4-chloro-1,2-diphenylbut-1-en- 1-yl)phenoxy)ethyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 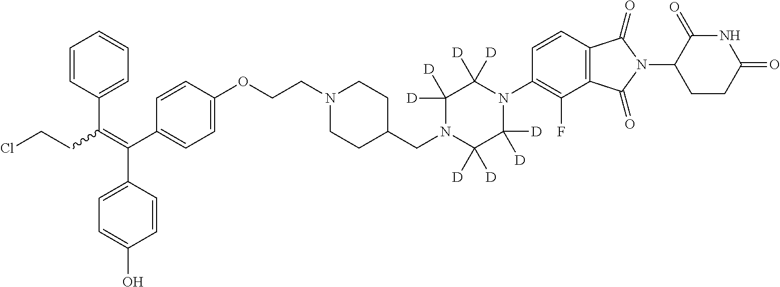 [SMILES: [2H]C1([2H])N(CC2CCN(CCOc3ccc(C(=C(CCCl)c4ccccc4)c4ccc(O)cc4)cc3)CC2)C([2H])([2H])C([2H])([2H])N(c2ccc3c(c2F)C(=O)N(C2CCC(=O)NC2=O)C3=O)C1([2H])[2H]] | 5-(4-((1-(2-(4-(4-chloro-1-(4-hydroxyphenyl)- 2-phenylbut-1-en-1- yl)phenoxy)ethyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 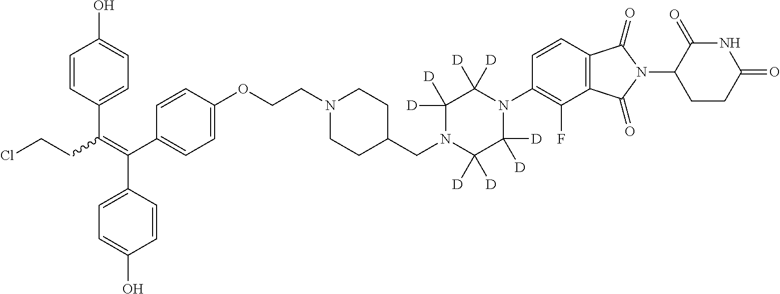 [SMILES: [2H]C1([2H])N(CC2CCN(CCOc3ccc(C(=C(CCCl)c4ccc(O)cc4)c4ccc(O)cc4)cc3)CC2)C([2H])([2H])C([2H])([2H])N(c2ccc3c(c2F)C(=O)N(C2CCC(=O)NC2=O)C3=O)C1([2H])[2H]] | 5-(4-((1-(2-(4-(4-chloro-1,2-bis(4- hydroxyphenyl)but-1-en-1- yl)phenoxy)ethyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 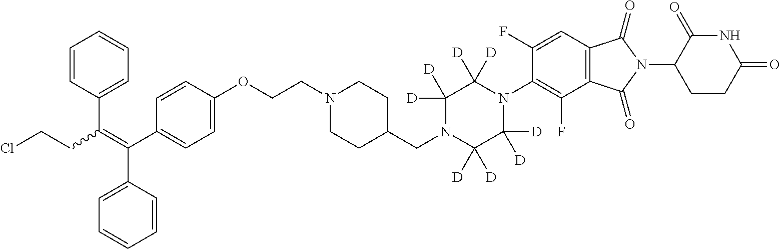 [SMILES: [2H]C1([2H])N(CC2CCN(CCOc3ccc(C(=C(CCCl)c4ccccc4)c4ccccc4)cc3)CC2)C([2H])([2H])C([2H])([2H])N(c2c(F)cc3c(c2F)C(=O)N(C2CCC(=O)NC2=O)C3=O)C1([2H])[2H]] | 5-(4-((1-(2-(4-(4-chloro-1,2-diphenylbut-1-en- 1-yl)phenoxy)ethyl)piperidin-4- yl)methyl)piperazin-1-yl-2,2,3,3,5,5,6,6-d₈)-2- (2,6-dioxopiperidin-3-yl)-4,6- difluoroisoindoline-1,3-dione |
| | 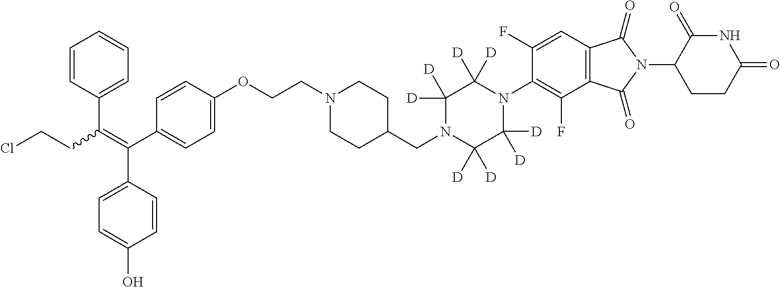 [SMILES: [2H]C1([2H])N(CC2CCN(CCOc3ccc(C(=C(CCCl)c4ccccc4)c4ccc(O)cc4)cc3)CC2)C([2H])([2H])C([2H])([2H])N(c2c(F)cc3c(c2F)C(=O)N(C2CCC(=O)NC2=O)C3=O)C1([2H])[2H]] | 5-(4-((1-(2-(4-(4-chloro-1-(4-hydroxyphenyl)- 2-phenylbut-1-en-1- yl)phenoxy)ethyl)piperidin-4- yl)methyl)piperazin-1-yl-2,2,3,3,5,5,6,6-d₈)-2- (2,6-dioxopiperidin-3-yl)-4,6- difluoroisoindoline-1,3-dione |
| | 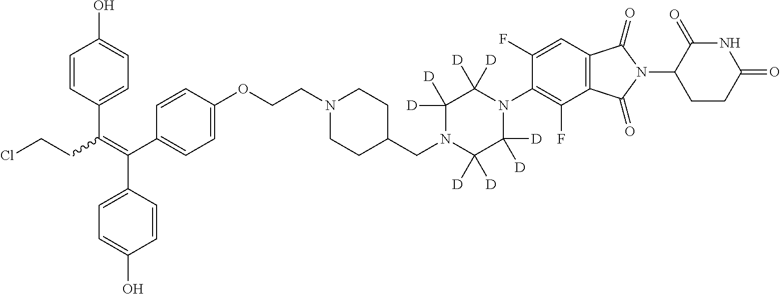 [SMILES: [2H]C1([2H])N(CC2CCN(CCOc3ccc(C(=C(CCCl)c4ccc(O)cc4)c4ccc(O)cc4)cc3)CC2)C([2H])([2H])C([2H])([2H])N(c2c(F)cc3c(c2F)C(=O)N(C2CCC(=O)NC2=O)C3=O)C1([2H])[2H]] | 5-(4-((1-(2-(4-(4-chloro-1,2-bis(4- hydroxyphenyl)but-1-en-1- yl)phenoxy)ethyl)piperidin-4- yl)methyl)piperazin-1-yl-2,2,3,3,5,5,6,6-d₈)-2- (2,6-dioxopiperidin-3-yl)-4,6- difluoroisoindoline-1,3-dione |
| | 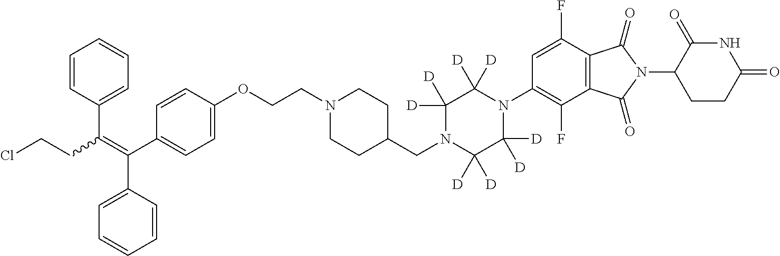 [SMILES: [2H]C1([2H])N(CC2CCN(CCOc3ccc(C(=C(CCCl)c4ccccc4)c4ccccc4)cc3)CC2)C([2H])([2H])C([2H])([2H])N(c2cc(F)c3c(c2F)C(=O)N(C2CCC(=O)NC2=O)C3=O)C1([2H])[2H]] | 5-(4-((1-(2-(4-(4-chloro-1,2-diphenylbut-1-en- 1-yl)phenoxy)ethyl)piperidin-4- yl)methyl)piperazin-1-yl-2,2,3,3,5,5,6,6-d₈)-2- (2,6-dioxopiperidin-3-yl)-4,7- difluoroisoindoline-1,3-dione |
| | 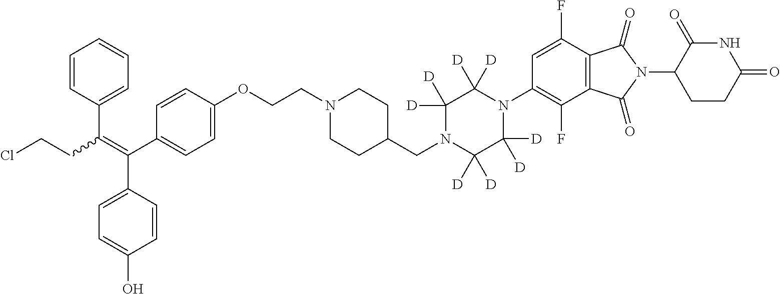 [SMILES: [2H]C1([2H])N(CC2CCN(CCOc3ccc(C(=C(CCCl)c4ccccc4)c4ccc(O)cc4)cc3)CC2)C([2H])([2H])C([2H])([2H])N(c2cc(F)c3c(c2F)C(=O)N(C2CCC(=O)NC2=O)C3=O)C1([2H])[2H]] | 5-(4-((1-(2-(4-(4-chloro-1-(4-hydroxyphenyl)- 2-phenylbut-1-en-1- yl)phenoxy)ethyl)piperidin-4- yl)methyl)piperazin-1-yl-2,2,3,3,5,5,6,6-d₈)-2- (2,6-dioxopiperidin-3-yl)-4,7- difluoroisoindoline-1,3-dione |
| | 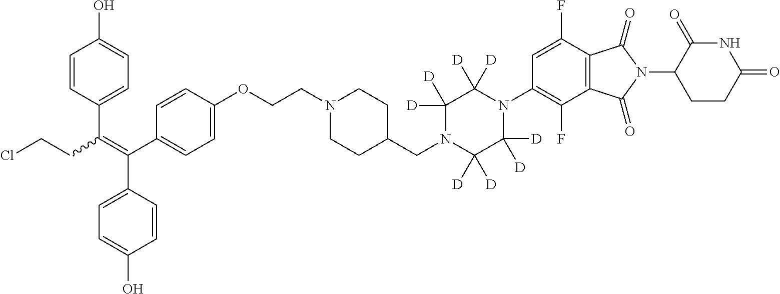 [SMILES: [2H]C1([2H])N(CC2CCN(CCOc3ccc(C(=C(CCCl)c4ccc(O)cc4)c4ccc(O)cc4)cc3)CC2)C([2H])([2H])C([2H])([2H])N(c2cc(F)c3c(c2F)C(=O)N(C2CCC(=O)NC2=O)C3=O)C1([2H])[2H]] | 5-(4-((1-(2-(4-(4-chloro-1,2-bis(4- hydroxyphenyl)but-1-en-1- yl)phenoxy)ethyl)piperidin-4- yl)methyl)piperazin-1-yl-2,2,3,3,5,5,6,6-d₈)-2- (2,6-dioxopiperidin-3-yl)-4,7- difluoroisoindoline-1,3-dione |
| | 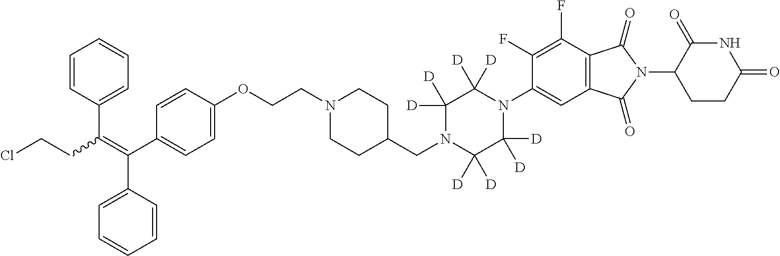 [SMILES: [2H]C1([2H])N(CC2CCN(CCOc3ccc(C(=C(CCCl)c4ccccc4)c4ccccc4)cc3)CC2)C([2H])([2H])C([2H])([2H])N(c2cc3c(c(F)c2F)C(=O)N(C2CCC(=O)NC2=O)C3=O)C1([2H])[2H]] | 6-(4-((1-(2-(4-(4-chloro-1,2-diphenylbut-1-en- 1-yl)phenoxy)ethyl)piperidin-4- yl)methyl)piperazin-1-yl-2,2,3,3,5,5,6,6-d₈)-2- (2,6-dioxopiperidin-3-yl)-4,5- difluoroisoindoline-1,3-dione |
| | 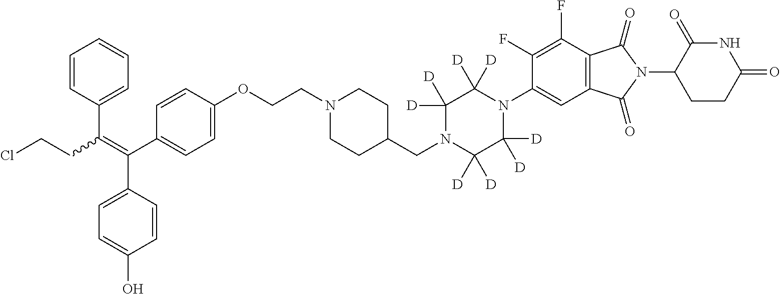 [SMILES: [2H]C1([2H])N(CC2CCN(CCOc3ccc(C(=C(CCCl)c4ccccc4)c4ccc(O)cc4)cc3)CC2)C([2H])([2H])C([2H])([2H])N(c2cc3c(c(F)c2F)C(=O)N(C2CCC(=O)NC2=O)C3=O)C1([2H])[2H]] | 6-(4-((1-(2-(4-(4-chloro-1-(4-hydroxyphenyl)- 2-phenylbut-1-en-1- yl)phenoxy)ethyl)piperidin-4- yl)methyl)piperazin-1-yl-2,2,3,3,5,5,6,6-d₈)-2- (2,6-dioxopiperidin-3-yl)-4,5- difluoroisoindoline-1,3-dione |
| | 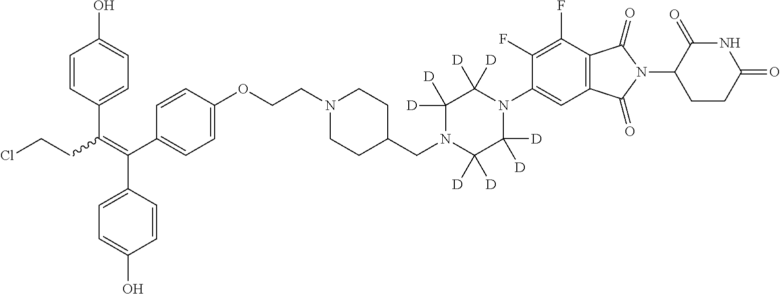 [SMILES: [2H]C1([2H])N(CC2CCN(CCOc3ccc(C(=C(CCCl)c4ccc(O)cc4)c4ccc(O)cc4)cc3)CC2)C([2H])([2H])C([2H])([2H])N(c2cc3c(c(F)c2F)C(=O)N(C2CCC(=O)NC2=O)C3=O)C1([2H])[2H]] | 6-(4-((1-(2-(4-(4-chloro-1,2-bis(4- hydroxyphenyl)but-1-en-1- yl)phenoxy)ethyl)piperidin-4- yl)methyl)piperazin-1-yl-2,2,3,3,5,5,6,6-d₈)-2- (2,6-dioxopiperidin-3-yl)-4,5- difluoroisoindoline-1,3-dione |
| | 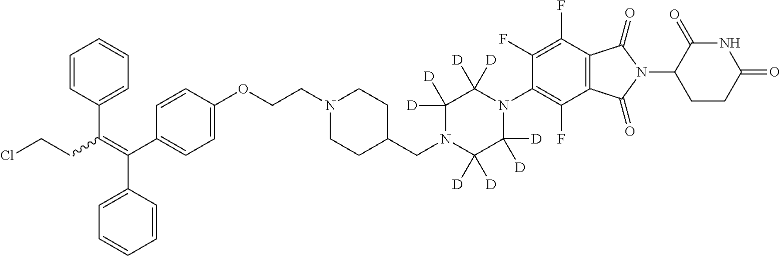 [SMILES: [2H]C1([2H])N(CC2CCN(CCOc3ccc(C(=C(CCCl)c4ccccc4)c4ccccc4)cc3)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(2-(4-(4-chloro-1,2-diphenylbut-1-en- 1-yl)phenoxy)ethyl)piperidin-4- yl)methyl)piperazin-1-yl-2,2,3,3,5,5,6,6-d₈)-2- (2,6-dioxopiperidin-3-yl)-4,6,7- trifluoroisoindoline-1,3-dione |
| | 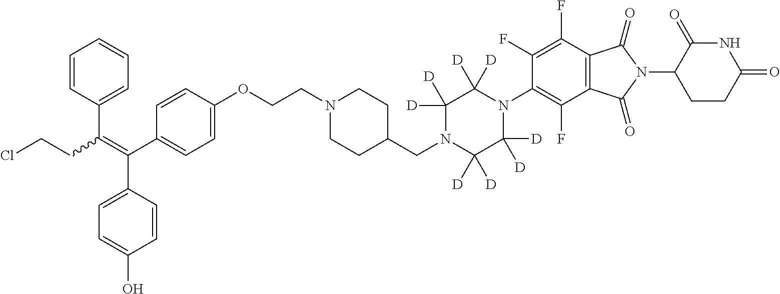 [SMILES: [2H]C1([2H])N(CC2CCN(CCOc3ccc(C(=C(CCCl)c4ccccc4)c4ccc(O)cc4)cc3)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(2-(4-(4-chloro-1-(4-hydroxyphenyl)- 2-phenylbut-1-en-1- yl)phenoxy)ethyl)piperidin-4- yl)methyl)piperazin-1-yl-2,2,3,3,5,5,6,6-d₈)-2- (2,6-dioxopiperidin-3-yl)-4,6,7- trifluoroisoindoline-1,3-dione |
| | 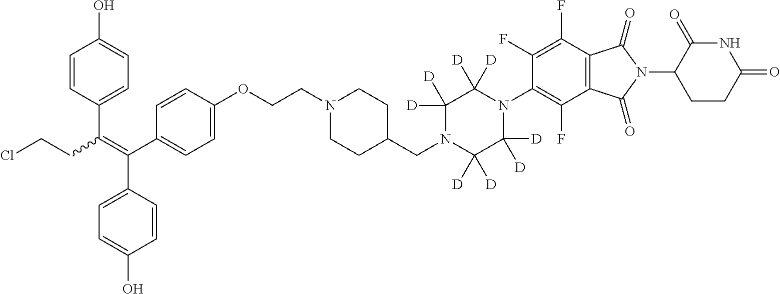 [SMILES: [2H]C1([2H])N(CC2CCN(CCOc3ccc(C(=C(CCCl)c4ccc(O)cc4)c4ccc(O)cc4)cc3)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(2-(4-(4-chloro-1,2-bis(4- hydroxyphenyl)but-1-en-1- yl)phenoxy)ethyl)piperidin-4- yl)methyl)piperazin-1-yl-2,2,3,3,5,5,6,6-d₈)-2- (2,6-dioxopiperidin-3-yl)-4,6,7- trifluoroisoindoline-1,3-dione |
| | 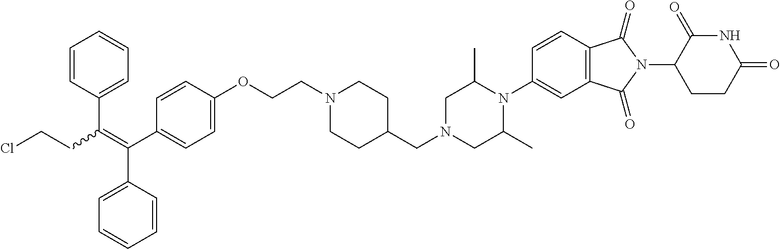 [SMILES: CC1CN(CC2CCN(CCOc3ccc(C(=C(CCCl)c4ccccc4)c4ccccc4)cc3)CC2)CC(C)N1c1ccc2c(c1)C(=O)N(C1CCC(=O)NC1=O)C2=O] | 5-(4-((1-(2-(4-(4-chloro-1,2-diphenylbut-1-en- 1-yl)phenoxy)ethyl)piperidin-4-yl)methyl)-2,6- dimethylpiperazin-1-yl)-2-(2,6-dioxopiperidin- 3-yl)isoindoline-1,3-dione |
| | 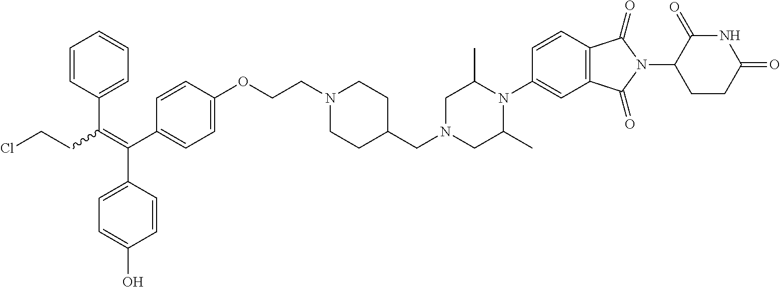 [SMILES: CC1CN(CC2CCN(CCOc3ccc(C(=C(CCCl)c4ccccc4)c4ccc(O)cc4)cc3)CC2)CC(C)N1c1ccc2c(c1)C(=O)N(C1CCC(=O)NC1=O)C2=O] | 5-(4-((1-(2-(4-(4-chloro-1-(4-hydroxyphenyl)- 2-phenylbut-1-en-1- yl)phenoxy)ethyl)piperidin-4-yl)methyl)-2,6- dimethylpiperazin-1-yl)-2-(2,6-dioxopiperidin- 3-yl)isoindoline-1,3-dione |
| | 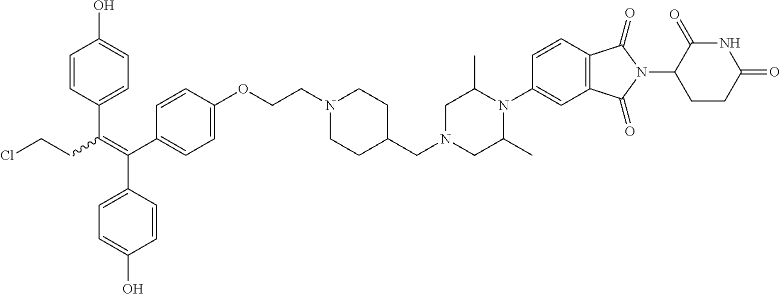 [SMILES: CC1CN(CC2CCN(CCOc3ccc(C(=C(CCCl)c4ccc(O)cc4)c4ccc(O)cc4)cc3)CC2)CC(C)N1c1ccc2c(c1)C(=O)N(C1CCC(=O)NC1=O)C2=O] | 5-(4-((1-(2-(4-(4-chloro-1,2-bis(4- hydroxyphenyl)but-1-en-1- yl)phenoxy)ethyl)piperidin-4-yl)methyl)-2,6- dimethylpiperazin-1-yl)-2-(2,6-dioxopiperidin- 3-yl)isoindoline-1,3-dione |
| | 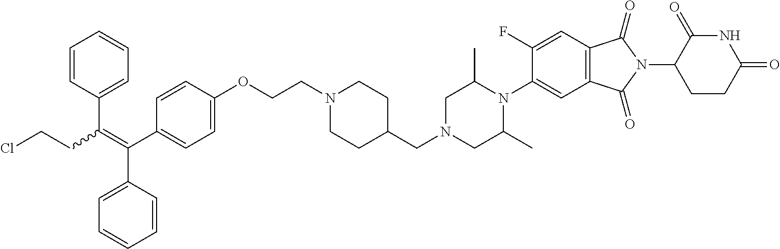 [SMILES: CC1CN(CC2CCN(CCOc3ccc(C(=C(CCCl)c4ccccc4)c4ccccc4)cc3)CC2)CC(C)N1c1cc2c(cc1F)C(=O)N(C1CCC(=O)NC1=O)C2=O] | 5-(4-((1-(2-(4-(4-chloro-1,2-diphenylbut-1-en- 1-yl)phenoxy)ethyl)piperidin-4-yl)methyl)-2,6- dimethylpiperazin-1-yl)-2-(2,6-dioxopiperidin- 3-yl)-6-fluoroisoindoline-1,3-dione |
| | 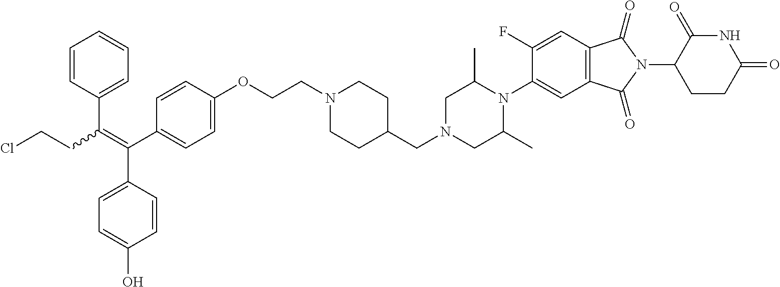 [SMILES: CC1CN(CC2CCN(CCOc3ccc(C(=C(CCCl)c4ccccc4)c4ccc(O)cc4)cc3)CC2)CC(C)N1c1cc2c(cc1F)C(=O)N(C1CCC(=O)NC1=O)C2=O] | 5-(4-((1-(2-(4-(4-chloro-1-(4-hydroxyphenyl)- 2-phenylbut-1-en-1- yl)phenoxy)ethyl)piperidin-4-yl)methyl)-2,6- dimethylpiperazin-1-yl)-2-(2,6-dioxopiperidin- 3-yl)-6-fluoroisoindoline-1,3-dione |
| | 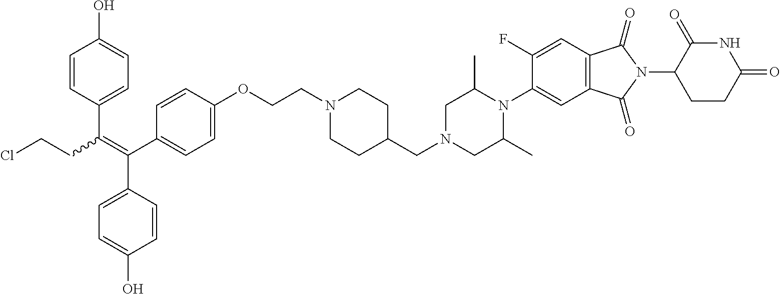 [SMILES: CC1CN(CC2CCN(CCOc3ccc(C(=C(CCCl)c4ccc(O)cc4)c4ccc(O)cc4)cc3)CC2)CC(C)N1c1cc2c(cc1F)C(=O)N(C1CCC(=O)NC1=O)C2=O] | 5-(4-((1-(2-(4-(4-chloro-1,2-bis(4- hydroxyphenyl)but-1-en-1- yl)phenoxy)ethyl)piperidin-4-yl)methyl)-2,6- dimethylpiperazin-1-yl)-2-(2,6-dioxopiperidin- 3-yl)-6-fluoroisoindoline-1,3-dione |
| | 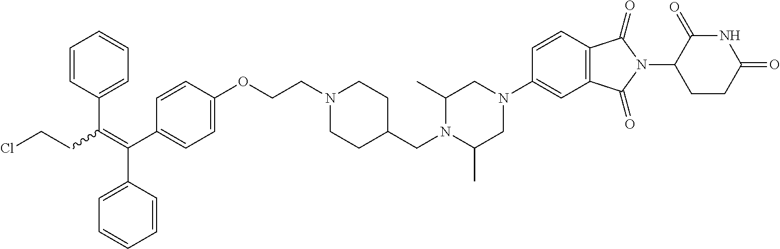 [SMILES: CC1CN(c2ccc3c(c2)C(=O)N(C2CCC(=O)NC2=O)C3=O)CC(C)N1CC1CCN(CCOc2ccc(C(=C(CCCl)c3ccccc3)c3ccccc3)cc2)CC1] | 5-(4-((1-(2-(4-(4-chloro-1,2-diphenylbut-1-en- 1-yl)phenoxy)ethyl)piperidin-4-yl)methyl)-3,5- dimethylpiperazin-1-yl)-2-(2,6-dioxopiperidin- 3-yl)isoindoline-1,3-dione |
| | 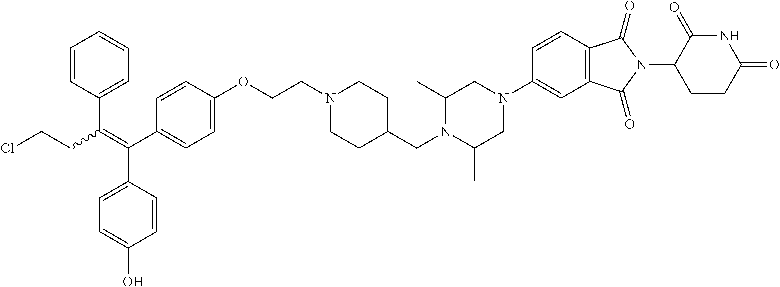 [SMILES: CC1CN(c2ccc3c(c2)C(=O)N(C2CCC(=O)NC2=O)C3=O)CC(C)N1CC1CCN(CCOc2ccc(C(=C(CCCl)c3ccccc3)c3ccc(O)cc3)cc2)CC1] | 5-(4-((1-(2-(4-(4-chloro-1-(4-hydroxyphenyl)- 2-phenylbut-1-en-1- yl)phenoxy)ethyl)piperidin-4-yl)methyl)-3,5- dimethylpiperazin-1-yl)-2-(2,6-dioxopiperidin- 3-yl)isoindoline-1,3-dione |
| | 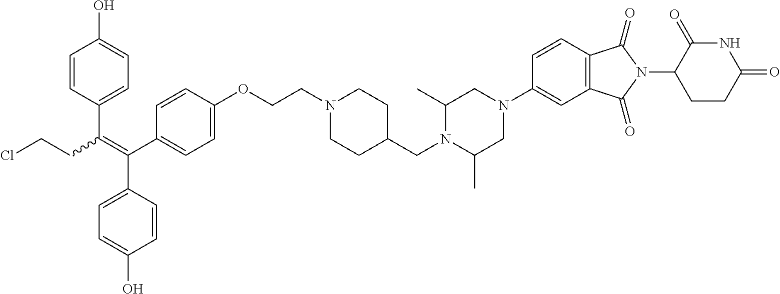 [SMILES: CC1CN(c2ccc3c(c2)C(=O)N(C2CCC(=O)NC2=O)C3=O)CC(C)N1CC1CCN(CCOc2ccc(C(=C(CCCl)c3ccc(O)cc3)c3ccc(O)cc3)cc2)CC1] | 5-(4-((1-(2-(4-(4-chloro-1,2-bis(4- hydroxyphenyl)but-1-en-1- yl)phenoxy)ethyl)piperidin-4-yl)methyl)-3,5- dimethylpiperazin-1-yl)-2-(2,6-dioxopiperidin- 3-yl)isoindoline-1,3-dione |
| | 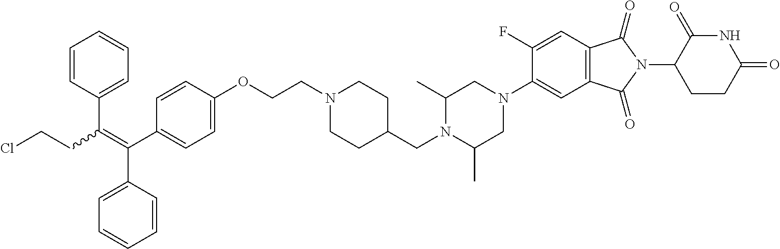 [SMILES: CC1CN(c2cc3c(cc2F)C(=O)N(C2CCC(=O)NC2=O)C3=O)CC(C)N1CC1CCN(CCOc2ccc(C(=C(CCCl)c3ccccc3)c3ccccc3)cc2)CC1] | 5-(4-((1-(2-(4-(4-chloro-1,2-diphenylbut-1-en- 1-yl)phenoxy)ethyl)piperidin-4-yl)methyl)-3,5- dimethylpiperazin-1-yl)-2-(2,6-dioxopiperidin- 3-yl)-6-fluoroisoindoline-1,3-dione |
| | 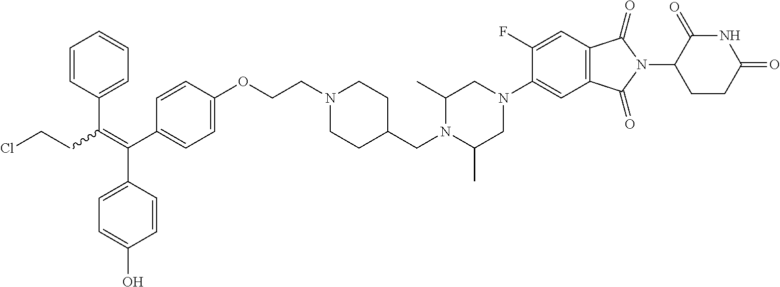 [SMILES: CC1CN(c2cc3c(cc2F)C(=O)N(C2CCC(=O)NC2=O)C3=O)CC(C)N1CC1CCN(CCOc2ccc(C(=C(CCCl)c3ccccc3)c3ccc(O)cc3)cc2)CC1] | 5-(4-((1-(2-(4-(4-chloro-1-(4-hydroxyphenyl)- 2-phenylbut-1-en-1- yl)phenoxy)ethyl)piperidin-4-yl)methyl)-3,5- dimethylpiperazin-1-yl)-2-(2,6-dioxopiperidin- 3-yl)-6-fluoroisoindoline-1,3-dione |
| | 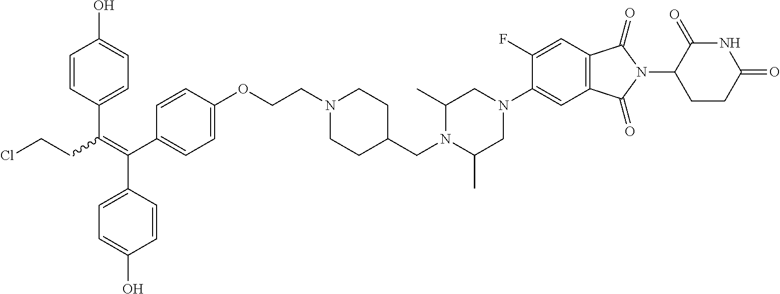 [SMILES: CC1CN(c2cc3c(cc2F)C(=O)N(C2CCC(=O)NC2=O)C3=O)CC(C)N1CC1CCN(CCOc2ccc(C(=C(CCCl)c3ccc(O)cc3)c3ccc(O)cc3)cc2)CC1] | 5-(4-((1-(2-(4-(4-chloro-1,2-bis(4- hydroxyphenyl)but-1-en-1- yl)phenoxy)ethyl)piperidin-4-yl)methyl)-3,5- dimethylpiperazin-1-yl)-2-(2,6-dioxopiperidin- 3-yl)-6-fluoroisoindoline-1,3-dione |
| | 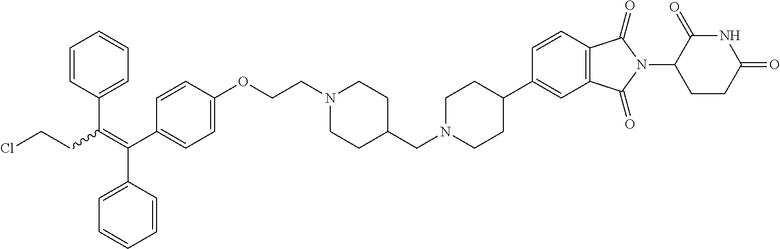 [SMILES: O=C1CCC(N2C(=O)c3ccc(C4CCN(CC5CCN(CCOc6ccc(C(=C(CCCl)c7ccccc7)c7ccccc7)cc6)CC5)CC4)cc3C2=O)C(=O)N1] | 5-(1-((1-(2-(4-(4-chloro-1,2-diphenylbut-1-en- 1-yl)phenoxy)ethyl)piperidin-4- yl)methyl)piperidin-4-yl)-2-(2,6- dioxopiperidin-3-yl)isoindoline-1,3-dione |
| | 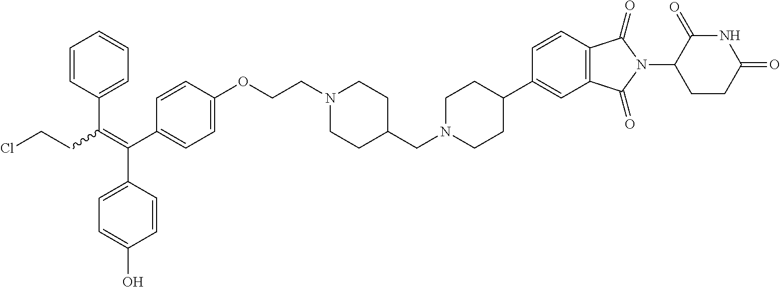 [SMILES: O=C1CCC(N2C(=O)c3ccc(C4CCN(CC5CCN(CCOc6ccc(C(=C(CCCl)c7ccccc7)c7ccc(O)cc7)cc6)CC5)CC4)cc3C2=O)C(=O)N1] | 5-(1-((1-(2-(4-(4-chloro-1-(4-hydroxyphenyl)- 2-phenylbut-1-en-1- yl)phenoxy)ethyl)piperidin-4- yl)methyl)piperidin-4-yl)-2-(2,6- dioxopiperidin-3-yl)isoindoline-1,3-dione |
| | 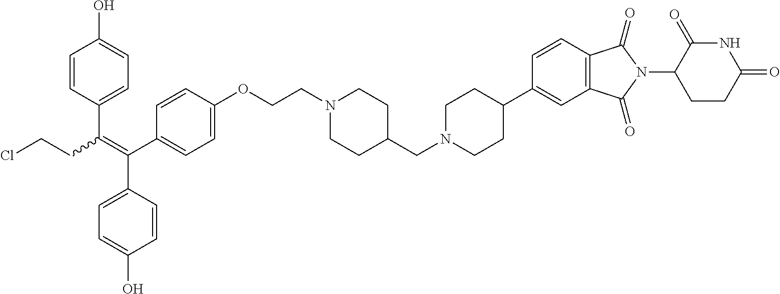 [SMILES: O=C1CCC(N2C(=O)c3ccc(C4CCN(CC5CCN(CCOc6ccc(C(=C(CCCl)c7ccc(O)cc7)c7ccc(O)cc7)cc6)CC5)CC4)cc3C2=O)C(=O)N1] | 5-(1-((1-(2-(4-(4-chloro-1,2-bis(4- hydroxyphenyl)but-1-en-1- yl)phenoxy)ethyl)piperidin-4- yl)methyl)piperidin-4-yl)-2-(2,6- dioxopiperidin-3-yl)isoindoline-1,3-dione |
| | 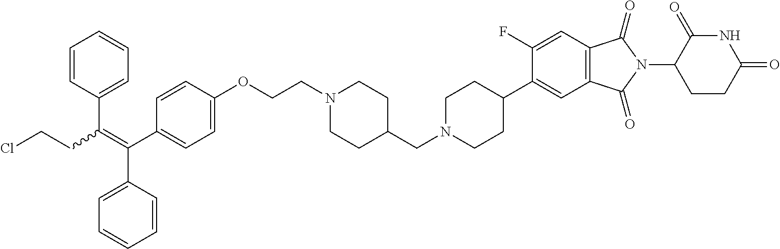 [SMILES: O=C1CCC(N2C(=O)c3cc(F)c(C4CCN(CC5CCN(CCOc6ccc(C(=C(CCCl)c7ccccc7)c7ccccc7)cc6)CC5)CC4)cc3C2=O)C(=O)N1] | 5-(1-((1-(2-(4-(4-chloro-1,2-diphenylbut-1-en- 1-yl)phenoxy)ethyl)piperidin-4- yl)methyl)piperidin-4-yl)-2-(2,6- dioxopiperidin-3-yl)-6-fluoroisoindoline-1,3- dione |
| | 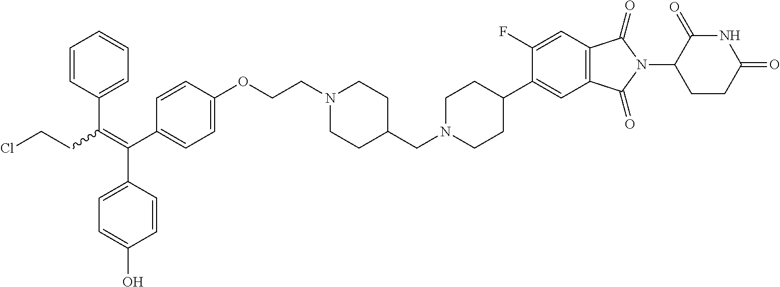 [SMILES: O=C1CCC(N2C(=O)c3cc(F)c(C4CCN(CC5CCN(CCOc6ccc(C(=C(CCCl)c7ccccc7)c7ccc(O)cc7)cc6)CC5)CC4)cc3C2=O)C(=O)N1] | 5-(1-((1-(2-(4-(4-chloro-1-(4-hydroxyphenyl)- 2-phenylbut-1-en-1- yl)phenoxy)ethyl)piperidin-4- yl)methyl)piperidin-4-yl)-2-(2,6- dioxopiperidin-3-yl)-6-fluoroisoindoline-1,3- dione |
| | 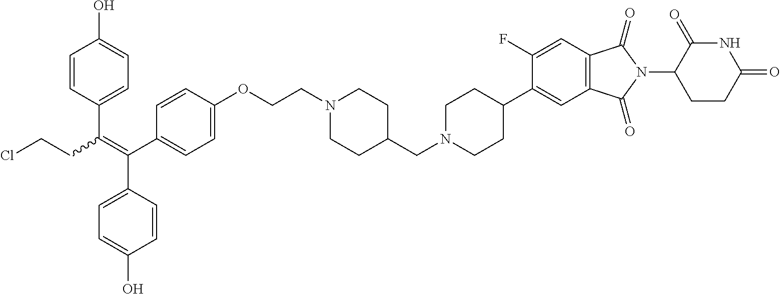 [SMILES: O=C1CCC(N2C(=O)c3cc(F)c(C4CCN(CC5CCN(CCOc6ccc(C(=C(CCCl)c7ccc(O)cc7)c7ccc(O)cc7)cc6)CC5)CC4)cc3C2=O)C(=O)N1] | 5-(1-((1-(2-(4-(4-chloro-1,2-bis(4- hydroxyphenyl)but-1-en-1- yl)phenoxy)ethyl)piperidin-4- yl)methyl)piperidin-4-yl)-2-(2,6- dioxopiperidin-3-yl)-6-fluoroisoindoline-1,3- dione |
| | 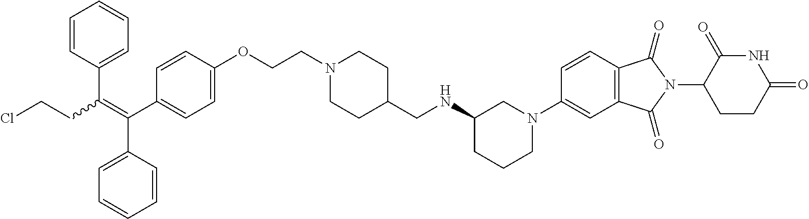 [SMILES: O=C1CCC(N2C(=O)c3ccc(N4CCC[C@@H](NCC5CCN(CCOc6ccc(C(=C(CCCl)c7ccccc7)c7ccccc7)cc6)CC5)C4)cc3C2=O)C(=O)N1] | 5-((R)-3-(((1-(2-(4-(4-chloro-1,2-diphenylbut- 1-en-1-yl)phenoxy)ethyl)piperidin-4- yl)methyl)amino)piperidin-1-yl)-2-(2,6- dioxopiperidin-3-yl)isoindoline-1,3-dione |
| | 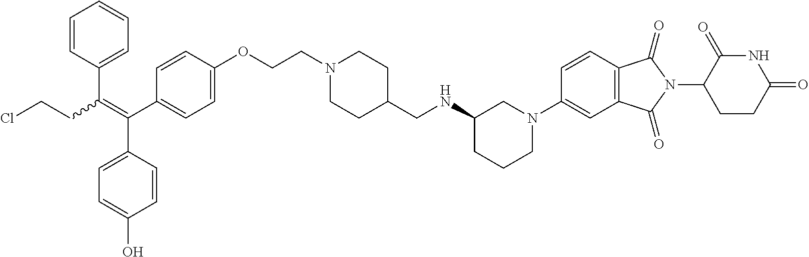 [SMILES: O=C1CCC(N2C(=O)c3ccc(N4CCC[C@@H](NCC5CCN(CCOc6ccc(C(=C(CCCl)c7ccccc7)c7ccc(O)cc7)cc6)CC5)C4)cc3C2=O)C(=O)N1] | 5-((R)-3-(((1-(2-(4-(4-chloro-1-(4- hydroxyphenyl)-2-phenylbut-1-en-1- yl)phenoxy)ethyl)piperidin-4- yl)methyl)amino)piperidin-1-yl)-2-(2,6- dioxopiperidin-3-yl)isoindoline-1,3-dione |
| | 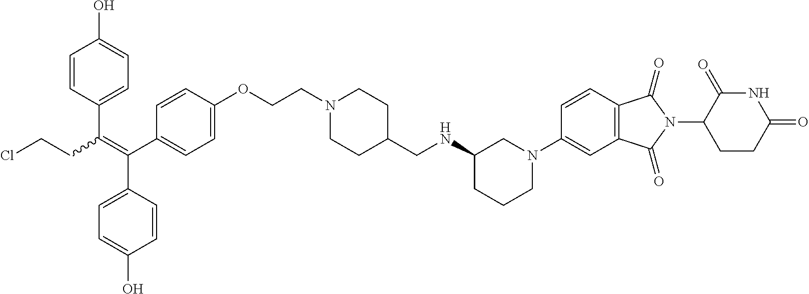 [SMILES: O=C1CCC(N2C(=O)c3ccc(N4CCC[C@@H](NCC5CCN(CCOc6ccc(C(=C(CCCl)c7ccc(O)cc7)c7ccc(O)cc7)cc6)CC5)C4)cc3C2=O)C(=O)N1] | 5-((R)-3-(((1-(2-(4-(4-chloro-1,2-bis(4- hydroxyphenyl)but-1-en-1- yl)phenoxy)ethyl)piperidin-4- yl)methyl)amino)piperidin-1-yl)-2-(2,6- dioxopiperidin-3-yl)isoindoline-1,3-dione |
| | 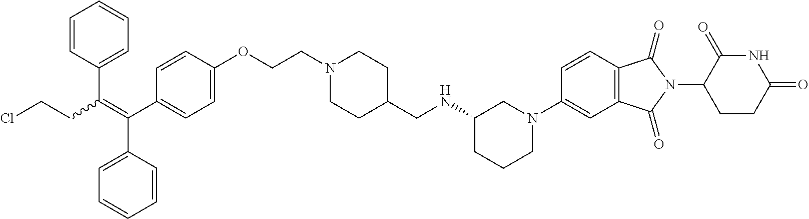 [SMILES: O=C1CCC(N2C(=O)c3ccc(N4CCC[C@H](NCC5CCN(CCOc6ccc(C(=C(CCCl)c7ccccc7)c7ccccc7)cc6)CC5)C4)cc3C2=O)C(=O)N1] | 5-((S)-3-(((1-(2-(4-(4-chloro-1,2-diphenylbut- 1-en-1-yl)phenoxy)ethyl)piperidin-4- yl)methyl)amino)piperidin-1-yl)-2-(2,6- dioxopiperidin-3-yl)isoindoline-1,3-dione |
| | 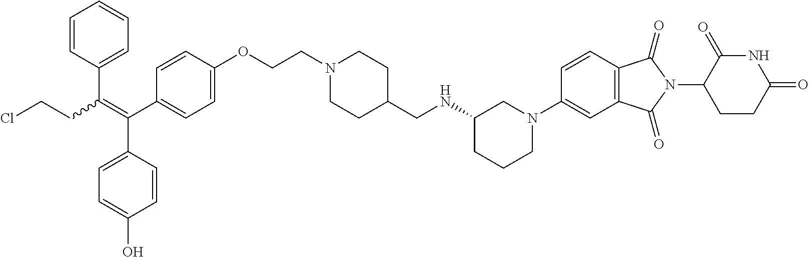 [SMILES: O=C1CCC(N2C(=O)c3ccc(N4CCC[C@H](NCC5CCN(CCOc6ccc(C(=C(CCCl)c7ccccc7)c7ccc(O)cc7)cc6)CC5)C4)cc3C2=O)C(=O)N1] | 5-((S)-3-(((1-(2-(4-(4-chloro-1-(4- hydroxyphenyl)-2-phenylbut-1-en-1- yl)phenoxy)ethyl)piperidin-4- yl)methyl)amino)piperidin-1-yl)-2-(2,6- dioxopiperidin-3-yl)isoindoline-1,3-dione |
| | 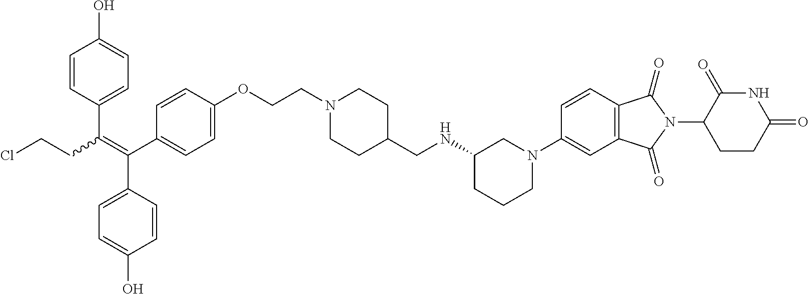 [SMILES: O=C1CCC(N2C(=O)c3ccc(N4CCC[C@H](NCC5CCN(CCOc6ccc(C(=C(CCCl)c7ccc(O)cc7)c7ccc(O)cc7)cc6)CC5)C4)cc3C2=O)C(=O)N1] | 5-((S)-3-(((1-(2-(4-(4-chloro-1,2-bis(4- hydroxyphenyl)but-1-en-1- yl)phenoxy)ethyl)piperidin-4- yl)methyl)amino)piperidin-1-yl)-2-(2,6- dioxopiperidin-3-yl)isoindoline-1,3-dione |
| | 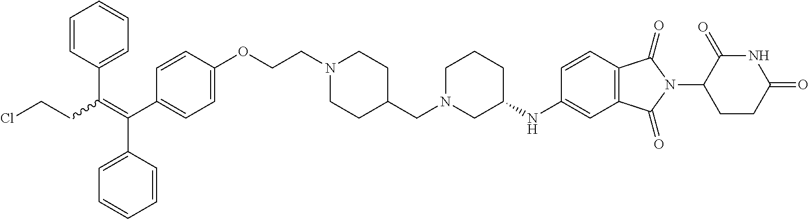 [SMILES: O=C1CCC(N2C(=O)c3ccc(N[C@H]4CCCN(CC5CCN(CCOc6ccc(C(=C(CCCl)c7ccccc7)c7ccccc7)cc6)CC5)C4)cc3C2=O)C(=O)N1] | 5-(((S)-1-((1-(2-(4-(4-chloro-1,2-diphenylbut- 1-en-1-yl)phenoxy)ethyl)piperidin-4- yl)methyl)piperidin-3-yl)amino)-2-(2,6- dioxopiperidin-3-yl)isoindoline-1,3-dione |
| | 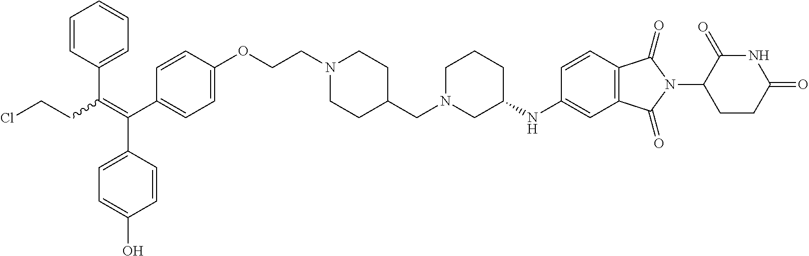 [SMILES: O=C1CCC(N2C(=O)c3ccc(N[C@H]4CCCN(CC5CCN(CCOc6ccc(C(=C(CCCl)c7ccccc7)c7ccc(O)cc7)cc6)CC5)C4)cc3C2=O)C(=O)N1] | 5-(((S)-1-((1-(2-(4-(4-chloro-1-(4- hydroxyphenyl)-2-phenylbut-1-en-1- yl)phenoxy)ethyl)piperidin-4- yl)methyl)piperidin-3-yl)amino)-2-(2,6- dioxopiperidin-3-yl)isoindoline-1,3-dione |
| | 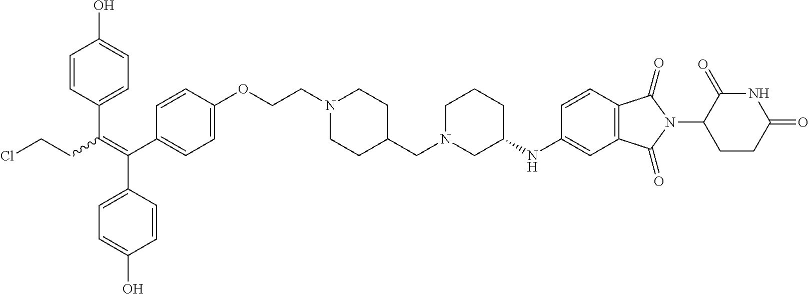 [SMILES: O=C1CCC(N2C(=O)c3ccc(N[C@H]4CCCN(CC5CCN(CCOc6ccc(C(=C(CCCl)c7ccc(O)cc7)c7ccc(O)cc7)cc6)CC5)C4)cc3C2=O)C(=O)N1] | 5-(((S)-1-((1-(2-(4-(4-chloro-1,2-bis(4- hydroxyphenyl)but-1-en-1- yl)phenoxy)ethyl)piperidin-4- yl)methyl)piperidin-3-yl)amino)-2-(2,6- dioxopiperidin-3-yl)isoindoline-1,3-dione |
| | 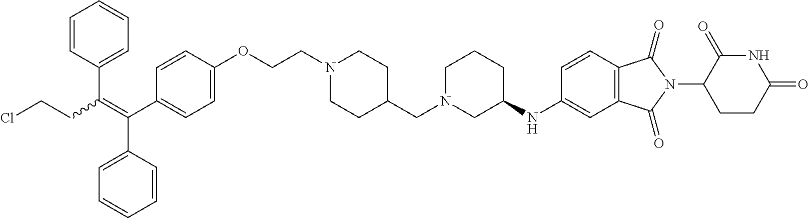 [SMILES: O=C1CCC(N2C(=O)c3ccc(N[C@@H]4CCCN(CC5CCN(CCOc6ccc(C(=C(CCCl)c7ccccc7)c7ccccc7)cc6)CC5)C4)cc3C2=O)C(=O)N1] | 5-(((R)-1-((1-(2-(4-(4-chloro-1,2-diphenylbut- 1-en-1-yl)phenoxy)ethyl)piperidin-4- yl)methyl)piperidin-3-yl)amino)-2-(2,6- dioxopiperidin-3-yl)isoindoline-1,3-dione |
| | 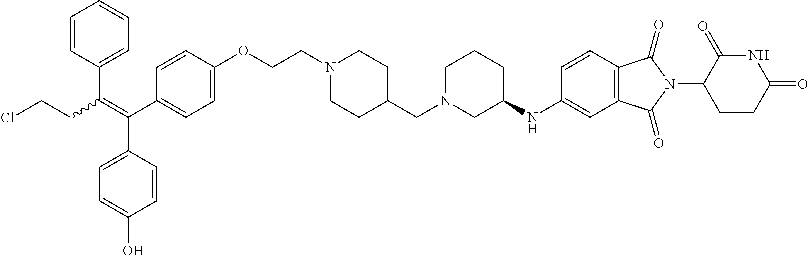 [SMILES: O=C1CCC(N2C(=O)c3ccc(N[C@@H]4CCCN(CC5CCN(CCOc6ccc(C(=C(CCCl)c7ccccc7)c7ccc(O)cc7)cc6)CC5)C4)cc3C2=O)C(=O)N1] | 5-(((R)-1-((1-(2-(4-(4-chloro-1-(4- hydroxyphenyl)-2-phenylbut-1-en-1- yl)phenoxy)ethyl)piperidin-4- yl)methyl)piperidin-3-yl)amino)-2-(2,6- dioxopiperidin-3-yl)isoindoline-1,3-dione |
| | 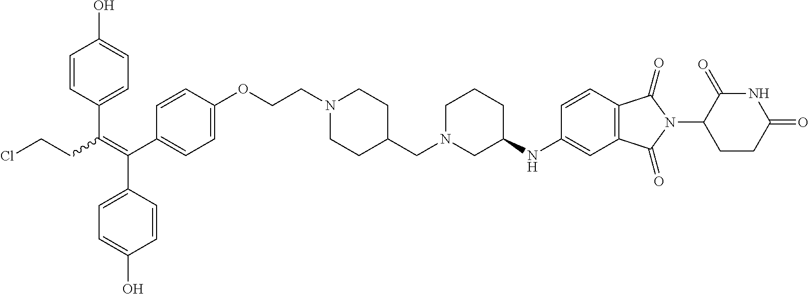 [SMILES: O=C1CCC(N2C(=O)c3ccc(N[C@@H]4CCCN(CC5CCN(CCOc6ccc(C(=C(CCCl)c7ccc(O)cc7)c7ccc(O)cc7)cc6)CC5)C4)cc3C2=O)C(=O)N1] | 5-(((R)-1-((1-(2-(4-(4-chloro-1,2-bis(4- hydroxyphenyl)but-1-en-1- yl)phenoxy)ethyl)piperidin-4- yl)methyl)piperidin-3-yl)amino)-2-(2,6- dioxopiperidin-3-yl)isoindoline-1,3-dione |
| | 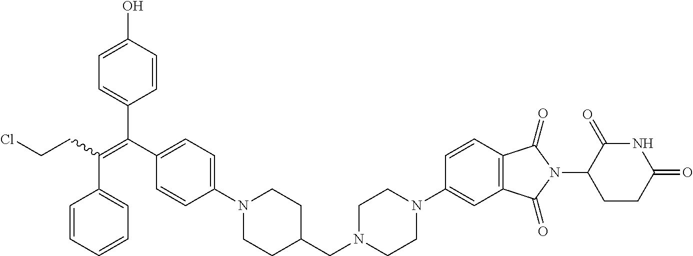 [SMILES: O=C1CCC(N2C(=O)c3ccc(N4CCN(CC5CCN(c6ccc(C(=C(CCCl)c7ccccc7)c7ccc(O)cc7)cc6)CC5)CC4)cc3C2=O)C(=O)N1] | 5-(4-((1-(4-(4-chloro-1-(4-hydroxyphenyl)-2- phenylbut-1-en-1-yl)phenyl)piperidin-4- yl)methyl)piperazin-1-yl)-2-(2,6- dioxopiperidin-3-yl)isoindoline-1,3-dione |
| | 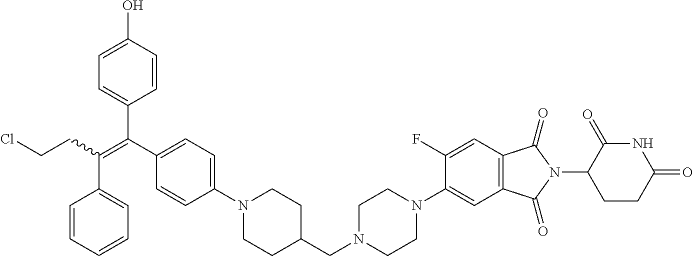 [SMILES: O=C1CCC(N2C(=O)c3cc(F)c(N4CCN(CC5CCN(c6ccc(C(=C(CCCl)c7ccccc7)c7ccc(O)cc7)cc6)CC5)CC4)cc3C2=O)C(=O)N1] | 5-(4-((1-(4-(4-chloro-1-(4-hydroxyphenyl)-2- phenylbut-1-en-1-yl)phenyl)piperidin-4- yl)methyl)piperazin-1-yl)-2-(2,6- dioxopiperidin-3-yl)-6-fluoroisoindoline-1,3- dione |
| | 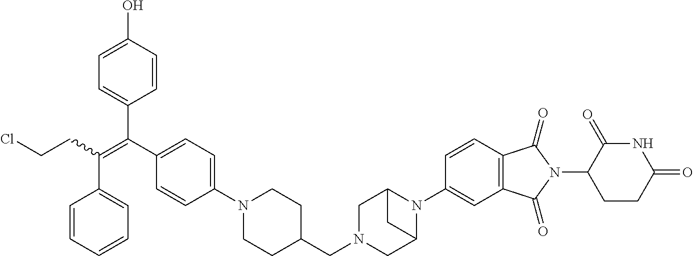 [SMILES: O=C1CCC(N2C(=O)c3ccc(N4C5CC4CN(CC4CCN(c6ccc(C(=C(CCCl)c7ccccc7)c7ccc(O)cc7)cc6)CC4)C5)cc3C2=O)C(=O)N1] | 5-(3-((1-(4-(4-chloro-1-(4-hydroxyphenyl)-2- phenylbut-1-en-1-yl)phenyl)piperidin-4- yl)methyl)-3,6-diazabicyclo[3.1.1]heptan-6-yl)- 2-(2,6-dioxopiperidin-3-yl)isoindoline-1,3- dione |
| | 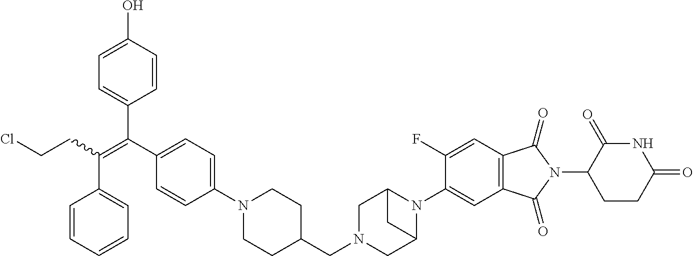 [SMILES: O=C1CCC(N2C(=O)c3cc(F)c(N4C5CC4CN(CC4CCN(c6ccc(C(=C(CCCl)c7ccccc7)c7ccc(O)cc7)cc6)CC4)C5)cc3C2=O)C(=O)N1] | 5-(3-((1-(4-(4-chloro-1-(4-hydroxyphenyl)-2- phenylbut-1-en-1-yl)phenyl)piperidin-4- yl)methyl)-3,6-diazabicyclo[3.1.1]heptan-6-yl)- 2-(2,6-dioxopiperidin-3-yl)-6- fluoroisoindoline-1,3-dione |
| | 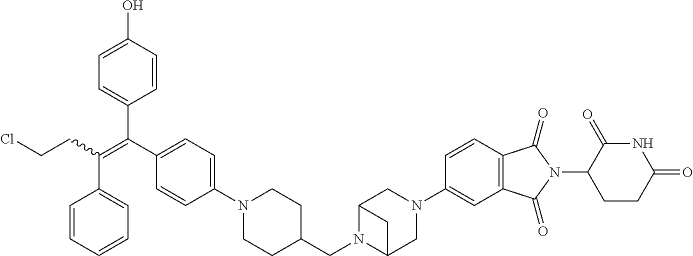 [SMILES: O=C1CCC(N2C(=O)c3ccc(N4CC5CC(C4)N5CC4CCN(c5ccc(C(=C(CCCl)c6ccccc6)c6ccc(O)cc6)cc5)CC4)cc3C2=O)C(=O)N1] | 5-(6-((1-(4-(4-chloro-1-(4-hydroxyphenyl)-2- phenylbut-1-en-1-yl)phenyl)piperidin-4- yl)methyl)-3,6-diazabicyclo[3.1.1]heptan-3-yl)- 2-(2,6-dioxopiperidin-3-yl)isoindoline-1,3- dione |
| | 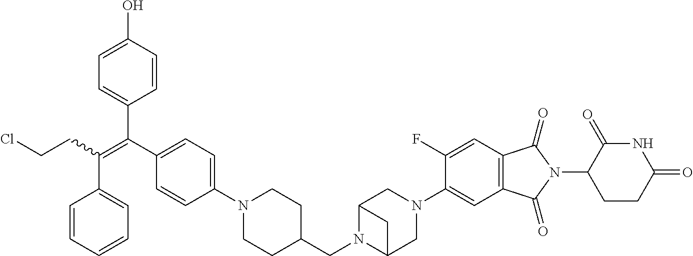 [SMILES: O=C1CCC(N2C(=O)c3cc(F)c(N4CC5CC(C4)N5CC4CCN(c5ccc(C(=C(CCCl)c6ccccc6)c6ccc(O)cc6)cc5)CC4)cc3C2=O)C(=O)N1] | 5-(6-((1-(4-(4-chloro-1-(4-hydroxyphenyl)-2- phenylbut-1-en-1-yl)phenyl)piperidin-4- yl)methyl)-3,6-diazabicyclo[3.1.1]heptan-3-yl)- 2-(2,6-dioxopiperidin-3-yl)-6- fluoroisoindoline-1,3-dione |
| | 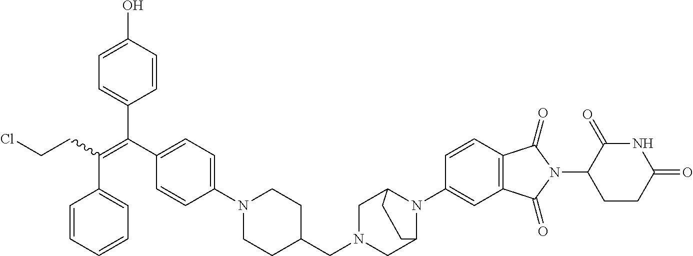 [SMILES: O=C1CCC(N2C(=O)c3ccc(N4C5CCC4CN(CC4CCN(c6ccc(C(=C(CCCl)c7ccccc7)c7ccc(O)cc7)cc6)CC4)C5)cc3C2=O)C(=O)N1] | 5-(3-((1-(4-(4-chloro-1-(4-hydroxyphenyl)-2- phenylbut-1-en-1-yl)phenyl)piperidin-4- yl)methyl)-3,8-diazabicyclo[3.2.1]octan-8-yl)- 2-(2,6-dioxopiperidin-3-yl)isoindoline-1,3- dione |
| | 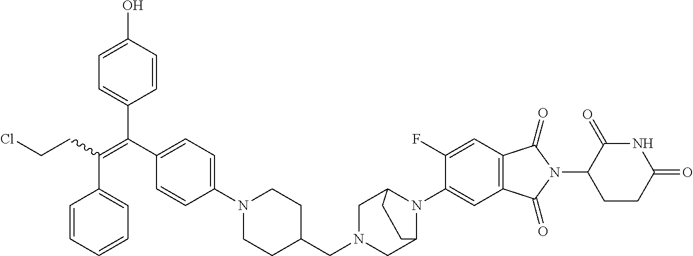 [SMILES: O=C1CCC(N2C(=O)c3cc(F)c(N4C5CCC4CN(CC4CCN(c6ccc(C(=C(CCCl)c7ccccc7)c7ccc(O)cc7)cc6)CC4)C5)cc3C2=O)C(=O)N1] | 5-(3-((1-(4-(4-chloro-1-(4-hydroxyphenyl)-2- phenylbut-1-en-1-yl)phenyl)piperidin-4- yl)methyl)-3,8-diazabicyclo[3.2.1]octan-8-yl)- 2-(2,6-dioxopiperidin-3-yl)-6- fluoroisoindoline-1,3-dione |
| | 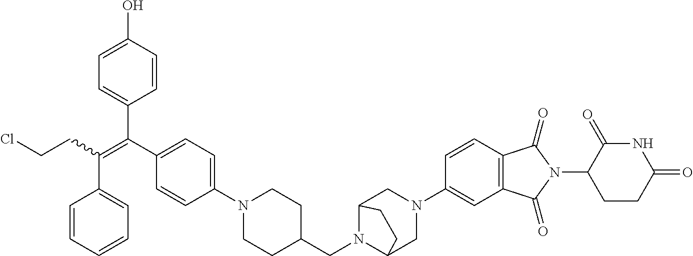 [SMILES: O=C1CCC(N2C(=O)c3ccc(N4CC5CCC(C4)N5CC4CCN(c5ccc(C(=C(CCCl)c6ccccc6)c6ccc(O)cc6)cc5)CC4)cc3C2=O)C(=O)N1] | 5-(8-((1-(4-(4-chloro-1-(4-hydroxyphenyl)-2- phenylbut-1-en-1-yl)phenyl)piperidin-4- yl)methyl)-3,8-diazabicyclo[3.2.1]octan-3-yl)- 2-(2,6-dioxopiperidin-3-yl)isoindoline-1,3- dione |
| | 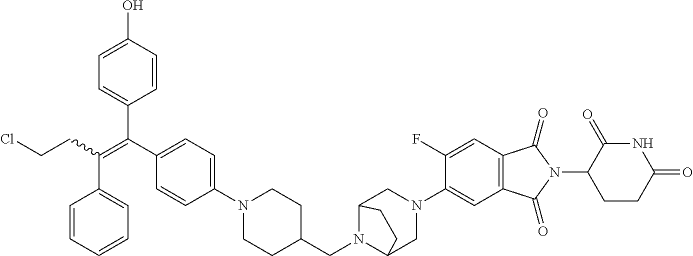 [SMILES: O=C1CCC(N2C(=O)c3cc(F)c(N4CC5CCC(C4)N5CC4CCN(c5ccc(C(=C(CCCl)c6ccccc6)c6ccc(O)cc6)cc5)CC4)cc3C2=O)C(=O)N1] | 5-(8-((1-(4-(4-chloro-1-(4-hydroxyphenyl)-2- phenylbut-1-en-1-yl)phenyl)piperidin-4- yl)methyl)-3,8-diazabicyclo[3.2.1]octan-3-yl)- 2-(2,6-dioxopiperidin-3-yl)-6- fluoroisoindoline-1,3-dione |
| | 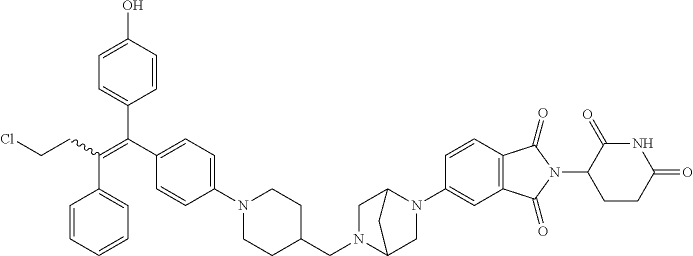 [SMILES: O=C1CCC(N2C(=O)c3ccc(N4CC5CC4CN5CC4CCN(c5ccc(C(=C(CCCl)c6ccccc6)c6ccc(O)cc6)cc5)CC4)cc3C2=O)C(=O)N1] | 5-(5-((1-(4-(4-chloro-1-(4-hydroxyphenyl)-2- phenylbut-1-en-1-yl)phenyl)piperidin-4- yl)methyl)-2,5-diazabicyclo[2.2.1]heptan-2-yl)- 2-(2,6-dioxopiperidin-3-yl)isoindoline-1,3- dione |
| | 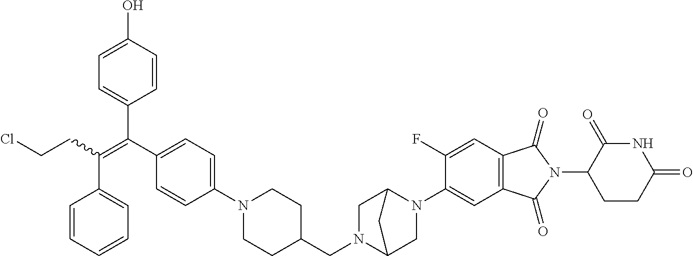 [SMILES: O=C1CCC(N2C(=O)c3cc(F)c(N4CC5CC4CN5CC4CCN(c5ccc(C(=C(CCCl)c6ccccc6)c6ccc(O)cc6)cc5)CC4)cc3C2=O)C(=O)N1] | 5-(5-((1-(4-(4-chloro-1-(4-hydroxyphenyl)-2- phenylbut-1-en-1-yl)phenyl)piperidin-4- yl)methyl)-2,5-diazabicyclo[2.2.1]heptan-2-yl)- 2-(2,6-dioxopiperidin-3-yl)-6- fluoroisoindoline-1,3-dione |
| | 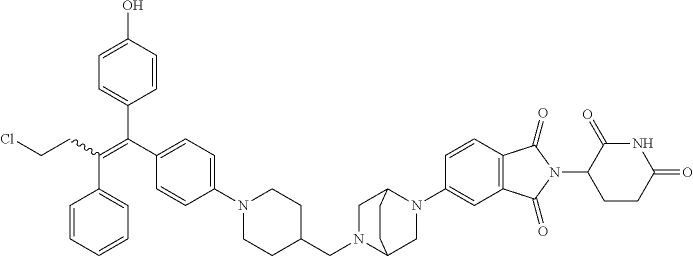 [SMILES: O=C1CCC(N2C(=O)c3ccc(N4CC5CCC4CN5CC4CCN(c5ccc(C(=C(CCCl)c6ccccc6)c6ccc(O)cc6)cc5)CC4)cc3C2=O)C(=O)N1] | 5-(5-((1-(4-(4-chloro-1-(4-hydroxyphenyl)-2- phenylbut-1-en-1-yl)phenyl)piperidin-4- yl)methyl)-2,5-diazabicyclo[2.2.2]octan-2-yl)- 2-(2,6-dioxopiperidin-3-yl)isoindoline-1,3- dione |
| | 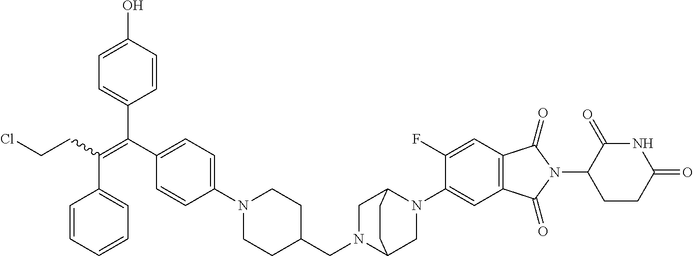 [SMILES: O=C1CCC(N2C(=O)c3cc(F)c(N4CC5CCC4CN5CC4CCN(c5ccc(C(=C(CCCl)c6ccccc6)c6ccc(O)cc6)cc5)CC4)cc3C2=O)C(=O)N1] | 5-(5-((1-(4-(4-chloro-1-(4-hydroxyphenyl)-2- phenylbut-1-en-1-yl)phenyl)piperidin-4- yl)methyl)-2,5-diazabicyclo[2.2.2]octan-2-yl)- 2-(2,6-dioxopiperidin-3-yl)-6- fluoroisoindoline-1,3-dione |
| | 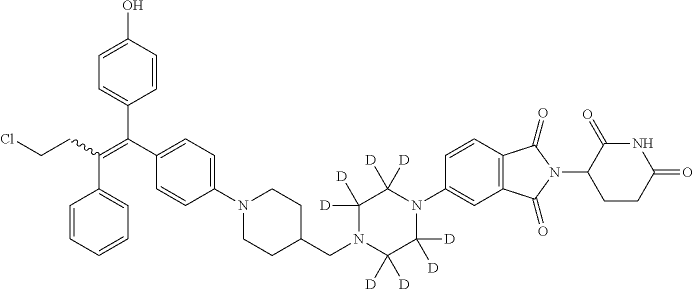 [SMILES: [2H]C1([2H])N(CC2CCN(c3ccc(C(=C(CCCl)c4ccccc4)c4ccc(O)cc4)cc3)CC2)C([2H])([2H])C([2H])([2H])N(c2ccc3c(c2)C(=O)N(C2CCC(=O)NC2=O)C3=O)C1([2H])[2H]] | 5-(4-((1-(4-(4-chloro-1-(4-hydroxyphenyl)-2- phenylbut-1-en-1-yl)phenyl)piperidin-4- yl)methyl)piperazin-1-yl-2,2,3,3,5,5,6,6-d₈)-2- (2,6-dioxopiperidin-3-yl)isoindoline-1,3-dione |
| | 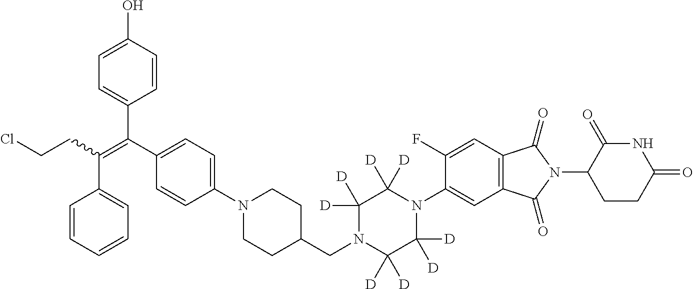 [SMILES: [2H]C1([2H])N(CC2CCN(c3ccc(C(=C(CCCl)c4ccccc4)c4ccc(O)cc4)cc3)CC2)C([2H])([2H])C([2H])([2H])N(c2cc3c(cc2F)C(=O)N(C2CCC(=O)NC2=O)C3=O)C1([2H])[2H]] | 5-(4-((1-(4-(4-chloro-1-(4-hydroxyphenyl)-2- phenylbut-1-en-1-yl)phenyl)piperidin-4- yl)methyl)piperazin-1-yl-2,2,3,3,5,5,6,6-d₈)-2- (2,6-dioxopiperidin-3-yl)-6-fluoroisoindoline- 1,3-dione |
| | 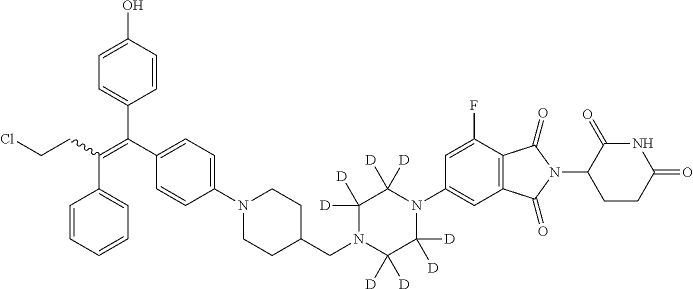 [SMILES: [2H]C1([2H])N(CC2CCN(c3ccc(C(=C(CCCl)c4ccccc4)c4ccc(O)cc4)cc3)CC2)C([2H])([2H])C([2H])([2H])N(c2cc(F)c3c(c2)C(=O)N(C2CCC(=O)NC2=O)C3=O)C1([2H])[2H]] | 6-(4-((1-(4-(4-chloro-1-(4-hydroxyphenyl)-2- phenylbut-1-en-1-yl)phenyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 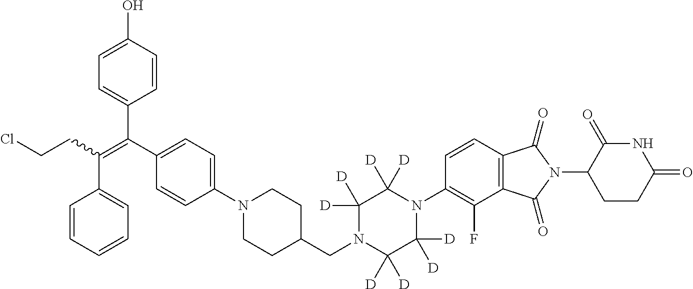 [SMILES: [2H]C1([2H])N(CC2CCN(c3ccc(C(=C(CCCl)c4ccccc4)c4ccc(O)cc4)cc3)CC2)C([2H])([2H])C([2H])([2H])N(c2ccc3c(c2F)C(=O)N(C2CCC(=O)NC2=O)C3=O)C1([2H])[2H]] | 5-(4-((1-(4-(4-chloro-1-(4-hydroxyphenyl)-2- phenylbut-1-en-1-yl)phenyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 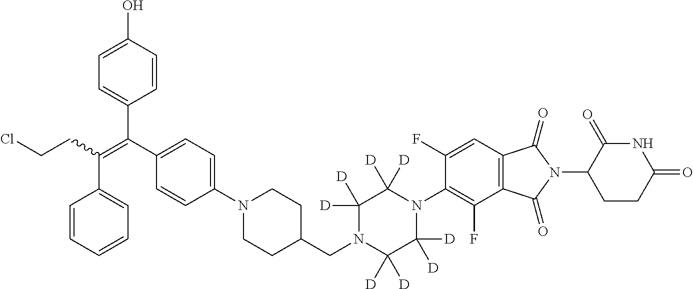 [SMILES: [2H]C1([2H])N(CC2CCN(c3ccc(C(=C(CCCl)c4ccccc4)c4ccc(O)cc4)cc3)CC2)C([2H])([2H])C([2H])([2H])N(c2c(F)cc3c(c2F)C(=O)N(C2CCC(=O)NC2=O)C3=O)C1([2H])[2H]] | 5-(4-((1-(4-(4-chloro-1-(4-hydroxyphenyl)-2- phenylbut-1-en-1-yl)phenyl)piperidin-4- yl)methyl)piperazin-1-yl-2,2,3,3,5,5,6,6-d₈)-2- (2,6-dioxopiperidin-3-yl)-4,6- difluoroisoindoline-1,3-dione |
| | 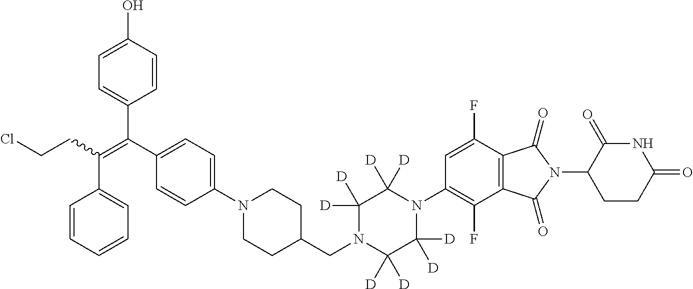 [SMILES: [2H]C1([2H])N(CC2CCN(c3ccc(C(=C(CCCl)c4ccccc4)c4ccc(O)cc4)cc3)CC2)C([2H])([2H])C([2H])([2H])N(c2cc(F)c3c(c2F)C(=O)N(C2CCC(=O)NC2=O)C3=O)C1([2H])[2H]] | 5-(4-((1-(4-(4-chloro-1-(4-hydroxyphenyl)-2- phenylbut-1-en-1-yl)phenyl)piperidin-4- yl)methyl)piperazin-1-yl-2,2,3,3,5,5,6,6-d₈)-2- (2,6-dioxopiperidin-3-yl)-4,7- difluoroisoindoline-1,3-dione |
| | 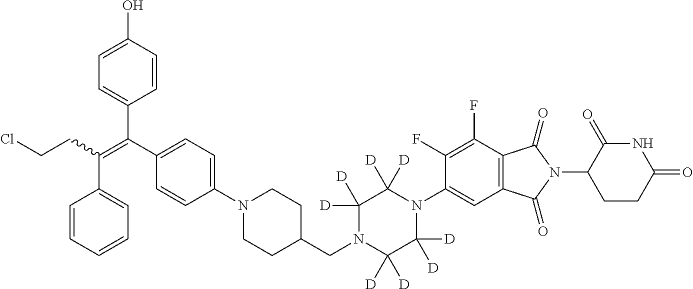 [SMILES: [2H]C1([2H])N(CC2CCN(c3ccc(C(=C(CCCl)c4ccccc4)c4ccc(O)cc4)cc3)CC2)C([2H])([2H])C([2H])([2H])N(c2cc3c(c(F)c2F)C(=O)N(C2CCC(=O)NC2=O)C3=O)C1([2H])[2H]] | 6-(4-((1-(4-(4-chloro-1-(4-hydroxyphenyl)-2- phenylbut-1-en-1-yl)phenyl)piperidin-4- yl)methyl)piperazin-1-yl-2,2,3,3,5,5,6,6-d₈)-2- (2,6-dioxopiperidin-3-yl)-4,5- difluoroisoindoline-1,3-dione |
| | 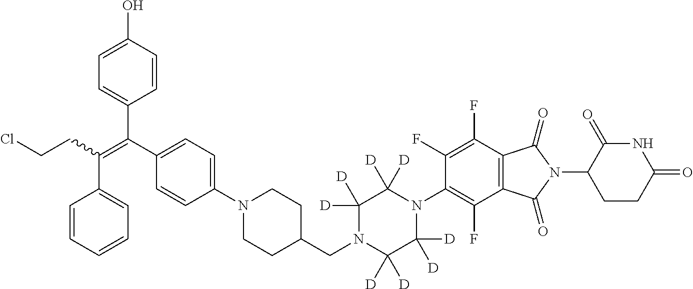 [SMILES: [2H]C1([2H])N(CC2CCN(c3ccc(C(=C(CCCl)c4ccccc4)c4ccc(O)cc4)cc3)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(4-(4-chloro-1-(4-hydroxyphenyl)-2- phenylbut-1-en-1-yl)phenyl)piperidin-4- yl)methyl)piperazin-1-yl-2,2,3,3,5,5,6,6-d₈)-2- (2,6-dioxopiperidin-3-yl)-4,6,7- trifluoroisoindoline-1,3-dione |
| | 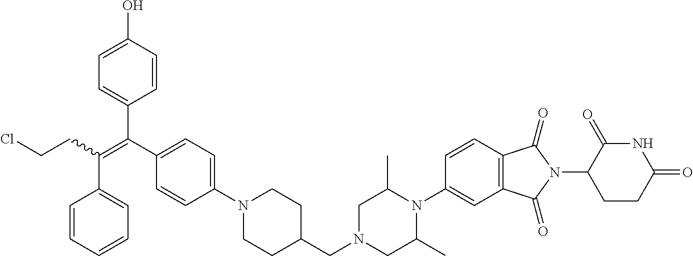 [SMILES: CC1CN(CC2CCN(c3ccc(C(=C(CCCl)c4ccccc4)c4ccc(O)cc4)cc3)CC2)CC(C)N1c1ccc2c(c1)C(=O)N(C1CCC(=O)NC1=O)C2=O] | 5-(4-((1-(4-(4-chloro-1-(4-hydroxyphenyl)-2- phenylbut-1-en-1-yl)phenyl)piperidin-4- yl)methyl)-2,6-dimethylpiperazin-1-yl)-2-(2,6- dioxopiperidin-3-yl)isoindoline-1,3-dione |
| | 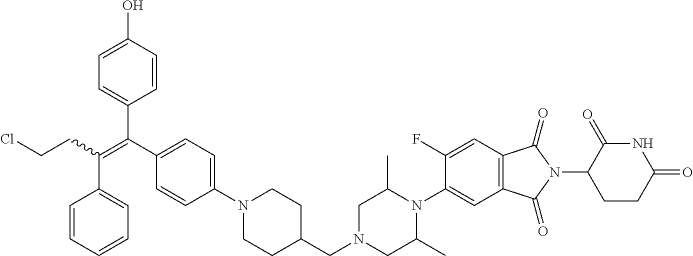 [SMILES: CC1CN(CC2CCN(c3ccc(C(=C(CCCl)c4ccccc4)c4ccc(O)cc4)cc3)CC2)CC(C)N1c1cc2c(cc1F)C(=O)N(C1CCC(=O)NC1=O)C2=O] | 5-(4-((1-(4-(4-chloro-1-(4-hydroxyphenyl)-2- phenylbut-1-en-1-yl)phenyl)piperidin-4- yl)methyl)-2,6-dimethylpiperazin-1-yl)-2-(2,6- dioxopiperidin-3-yl)-6-fluoroisoindoline-1,3- dione |
| | 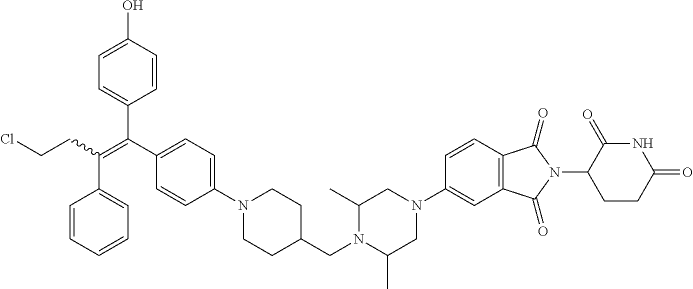 [SMILES: CC1CN(c2ccc3c(c2)C(=O)N(C2CCC(=O)NC2=O)C3=O)CC(C)N1CC1CCN(c2ccc(C(=C(CCCl)c3ccccc3)c3ccc(O)cc3)cc2)CC1] | 5-(4-((1-(4-(4-chloro-1-(4-hydroxyphenyl)-2- phenylbut-1-en-1-yl)phenyl)piperidin-4- yl)methyl)-3,5-dimethylpiperazin-1-yl)-2-(2,6- dioxopiperidin-3-yl)isoindoline-1,3-dione |
| | 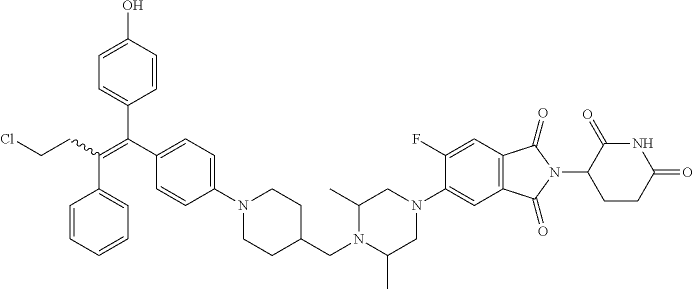 [SMILES: CC1CN(c2cc3c(cc2F)C(=O)N(C2CCC(=O)NC2=O)C3=O)CC(C)N1CC1CCN(c2ccc(C(=C(CCCl)c3ccccc3)c3ccc(O)cc3)cc2)CC1] | 5-(4-((1-(4-(4-chloro-1-(4-hydroxyphenyl)-2- phenylbut-1-en-1-yl)phenyl)piperidin-4- yl)methyl)-3,5-dimethylpiperazin-1-yl)-2-(2,6- dioxopiperidin-3-yl)-6-fluoroisoindoline-1,3- dione |
| | 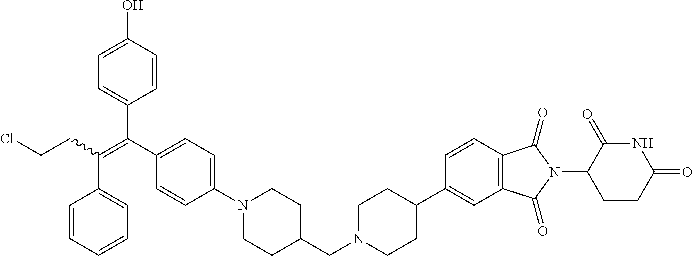 [SMILES: O=C1CCC(N2C(=O)c3ccc(C4CCN(CC5CCN(c6ccc(C(=C(CCCl)c7ccccc7)c7ccc(O)cc7)cc6)CC5)CC4)cc3C2=O)C(=O)N1] | 5-(1-((1-(4-(4-chloro-1-(4-hydroxyphenyl)-2- phenylbut-1-en-1-yl)phenyl)piperidin-4- yl)methyl)piperidin-4-yl)-2-(2,6- dioxopiperidin-3-yl)isoindoline-1,3-dione |
| | 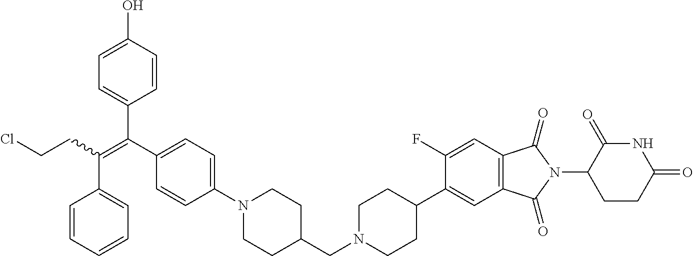 [SMILES: O=C1CCC(N2C(=O)c3cc(F)c(C4CCN(CC5CCN(c6ccc(C(=C(CCCl)c7ccccc7)c7ccc(O)cc7)cc6)CC5)CC4)cc3C2=O)C(=O)N1] | 5-(1-((1-(4-(4-chloro-1-(4-hydroxyphenyl)-2- phenylbut-1-en-1-yl)phenyl)piperidin-4- yl)methyl)piperidin-4-yl)-2-(2,6- dioxopiperidin-3-yl)-6-fluoroisoindoline-1,3- dione |
| | 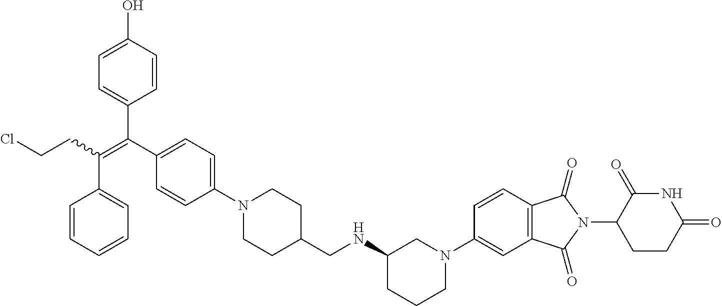 [SMILES: O=C1CCC(N2C(=O)c3ccc(N4CCC[C@@H](NCC5CCN(c6ccc(C(=C(CCCl)c7ccccc7)c7ccc(O)cc7)cc6)CC5)C4)cc3C2=O)C(=O)N1] | 5-((R)-3-(((1-(4-(4-chloro-1-(4- hydroxyphenyl)-2-phenylbut-1-en-1- yl)phenyl)piperidin-4- yl)methyl)amino)piperidin-1-yl)-2-(2,6- dioxopiperidin-3-yl)isoindoline-1,3-dione |
| | 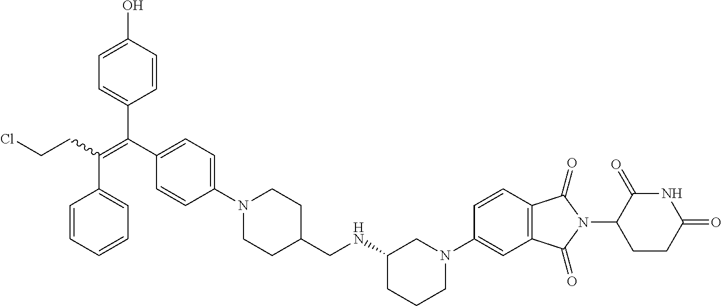 [SMILES: O=C1CCC(N2C(=O)c3ccc(N4CCC[C@H](NCC5CCN(c6ccc(C(=C(CCCl)c7ccccc7)c7ccc(O)cc7)cc6)CC5)C4)cc3C2=O)C(=O)N1] | 5-((S)-3-(((1-(4-(4-chloro-1-(4- hydroxyphenyl)-2-phenylbut-1-en-1- yl)phenyl)piperidin-4- yl)methyl)amino)piperidin-1-yl)-2-(2,6- dioxopiperidin-3-yl)isoindoline-1,3-dione |
| | 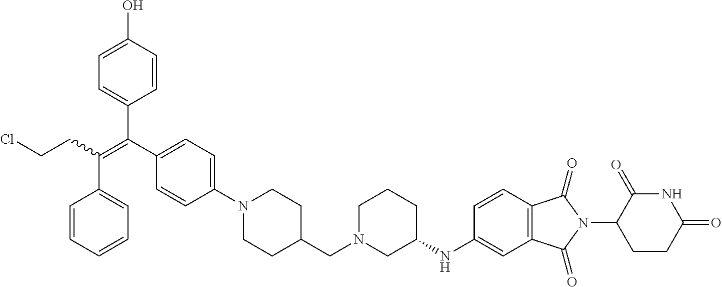 [SMILES: O=C1CCC(N2C(=O)c3ccc(N[C@H]4CCCN(CC5CCN(c6ccc(C(=C(CCCl)c7ccccc7)c7ccc(O)cc7)cc6)CC5)C4)cc3C2=O)C(=O)N1] | 5-(((S)-1-((1-(4-(4-chloro-1-(4- hydroxyphenyl)-2-phenylbut-1-en-1- yl)phenyl)piperidin-4-yl)methyl)piperidin-3- yl)amino)-2-(2,6-dioxopiperidin-3- yl)isoindoline-1,3-dione |
| | 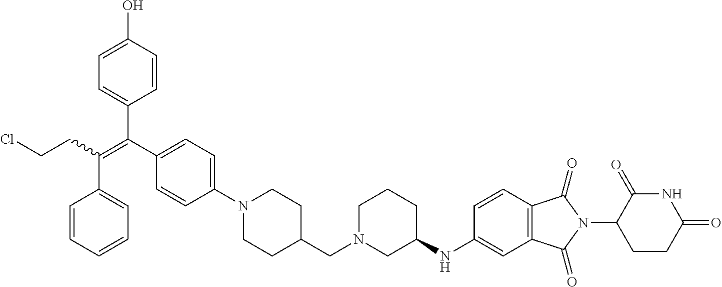 [SMILES: O=C1CCC(N2C(=O)c3ccc(N[C@@H]4CCCN(CC5CCN(c6ccc(C(=C(CCCl)c7ccccc7)c7ccc(O)cc7)cc6)CC5)C4)cc3C2=O)C(=O)N1] | 5-(((R)-1-((1-(4-(4-chloro-1-(4- hydroxyphenyl)-2-phenylbut-1-en-1- yl)phenyl)piperidin-4-yl)methyl)piperidin-3- yl)amino)-2-(2,6-dioxopiperidin-3- yl)isoindoline-1,3-dione |
| | 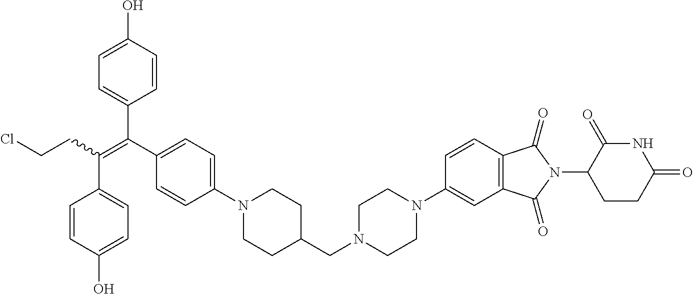 [SMILES: O=C1CCC(N2C(=O)c3ccc(N4CCN(CC5CCN(c6ccc(C(=C(CCCl)c7ccc(O)cc7)c7ccc(O)cc7)cc6)CC5)CC4)cc3C2=O)C(=O)N1] | 5-(4-((1-(4-(4-chloro-1,2-bis(4- hydroxyphenyl)but-1-en-1-yl)phenyl)piperidin- 4-yl)methyl)piperazin-1-yl)-2-(2,6- dioxopiperidin-3-yl)isoindoline-1,3-dione |
| | 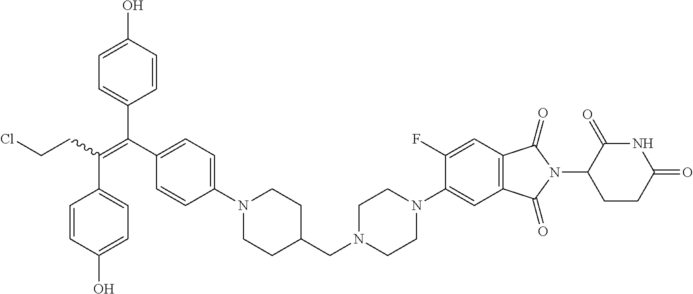 [SMILES: O=C1CCC(N2C(=O)c3cc(F)c(N4CCN(CC5CCN(c6ccc(C(=C(CCCl)c7ccc(O)cc7)c7ccc(O)cc7)cc6)CC5)CC4)cc3C2=O)C(=O)N1] | 5-(4-((1-(4-(4-chloro-1,2-bis(4- hydroxyphenyl)but-1-en-1-yl)phenyl)piperidin- 4-yl)methyl)piperazin-1-yl)-2-(2,6- dioxopiperidin-3-yl)-6-fluoroisoindoline-1,3- dione |
| | 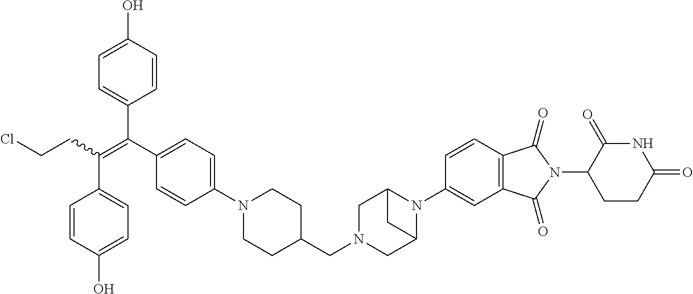 [SMILES: O=C1CCC(N2C(=O)c3ccc(N4C5CC4CN(CC4CCN(c6ccc(C(=C(CCCl)c7ccc(O)cc7)c7ccc(O)cc7)cc6)CC4)C5)cc3C2=O)C(=O)N1] | 5-(3-((1-(4-(4-chloro-1,2-bis(4- hydroxyphenyl)but-1-en-1-yl)phenyl)piperidin- 4-yl)methyl)-3,6-diazabicyclo[3.1.1]heptan-6- yl)-2-(2,6-dioxopiperidin-3-yl)isoindoline-1,3- dione |
| | 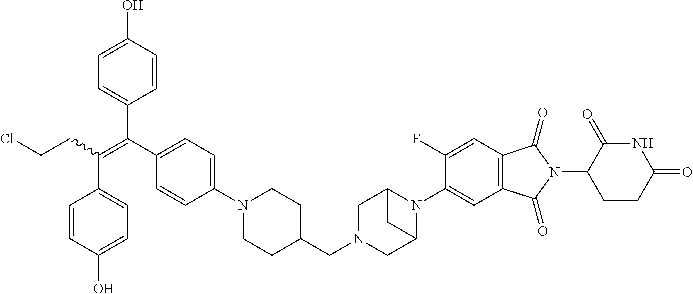 [SMILES: O=C1CCC(N2C(=O)c3cc(F)c(N4C5CC4CN(CC4CCN(c6ccc(C(=C(CCCl)c7ccc(O)cc7)c7ccc(O)cc7)cc6)CC4)C5)cc3C2=O)C(=O)N1] | 5-(3-((1-(4-(4-chloro-1,2-bis(4- hydroxyphenyl)but-1-en-1-yl)phenyl)piperidin- 4-yl)methyl)-3,6-diazabicyclo[3.1.1]heptan-6- yl)-2-(2,6-dioxopiperidin-3-yl)-6- fluoroisoindoline-1,3-dione |
| | 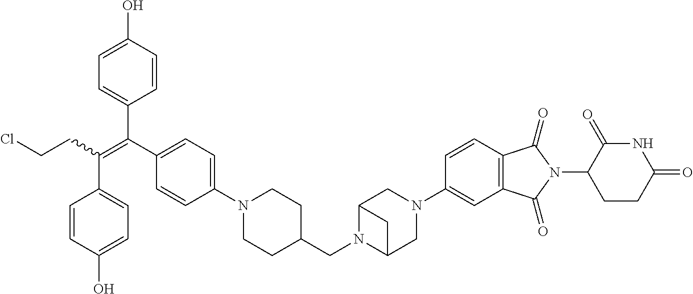 [SMILES: O=C1CCC(N2C(=O)c3ccc(N4CC5CC(C4)N5CC4CCN(c5ccc(C(=C(CCCl)c6ccc(O)cc6)c6ccc(O)cc6)cc5)CC4)cc3C2=O)C(=O)N1] | 5-(6-((1-(4-(4-chloro-1,2-bis(4- hydroxyphenyl)but-1-en-1-yl)phenyl)piperidin- 4-yl)methyl)-3,6-diazabicyclo[3.1.1]heptan-3- yl)-2-(2,6-dioxopiperidin-3-yl)isoindoline-1,3- dione |
| | 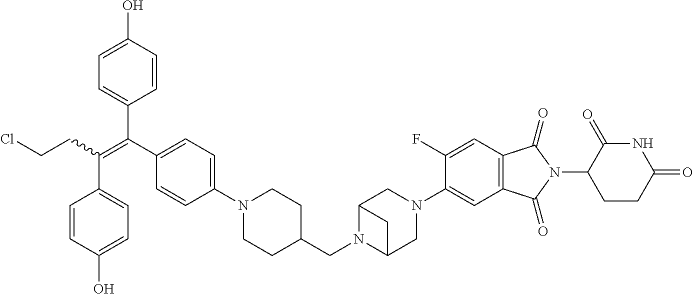 [SMILES: O=C1CCC(N2C(=O)c3cc(F)c(N4CC5CC(C4)N5CC4CCN(c5ccc(C(=C(CCCl)c6ccc(O)cc6)c6ccc(O)cc6)cc5)CC4)cc3C2=O)C(=O)N1] | 5-(6-((1-(4-(4-chloro-1,2-bis(4- hydroxyphenyl)but-1-en-1-yl)phenyl)piperidin- 4-yl)methyl)-3,6-diazabicyclo[3.1.1]heptan-3- yl)-2-(2,6-dioxopiperidin-3-yl)-6- fluoroisoindoline-1,3-dione |
| | 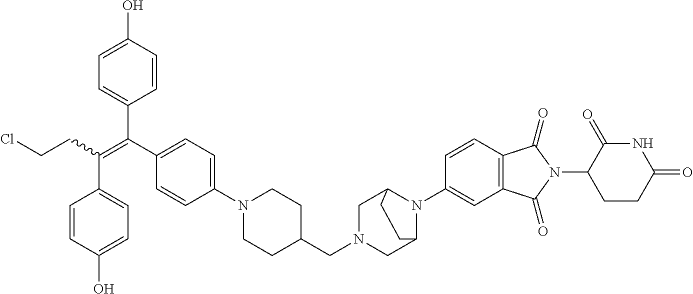 [SMILES: O=C1CCC(N2C(=O)c3ccc(N4C5CCC4CN(CC4CCN(c6ccc(C(=C(CCCl)c7ccc(O)cc7)c7ccc(O)cc7)cc6)CC4)C5)cc3C2=O)C(=O)N1] | 5-(3-((1-(4-(4-chloro-1,2-bis(4- hydroxyphenyl)but-1-en-1-yl)phenyl)piperidin- 4-yl)methyl)-3,8-diazabicyclo[3.2.1]octan-8- yl)-2-(2,6-dioxopiperidin-3-yl)isoindoline-1,3- dione |
| | 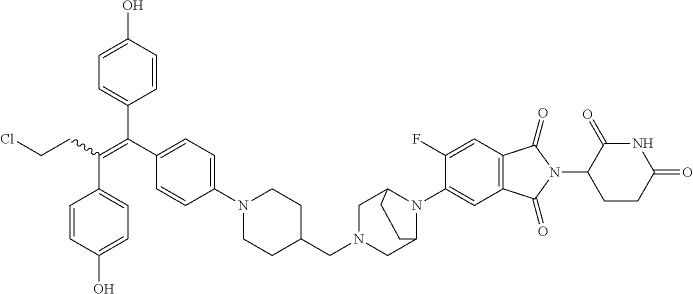 [SMILES: O=C1CCC(N2C(=O)c3cc(F)c(N4C5CCC4CN(CC4CCN(c6ccc(C(=C(CCCl)c7ccc(O)cc7)c7ccc(O)cc7)cc6)CC4)C5)cc3C2=O)C(=O)N1] | 5-(3-((1-(4-(4-chloro-1,2-bis(4- hydroxyphenyl)but-1-en-1-yl)phenyl)piperidin- 4-yl)methyl)-3,8-diazabicyclo[3.2.1]octan-8- yl)-2-(2,6-dioxopiperidin-3-yl)-6- fluoroisoindoline-1,3-dione |
| | 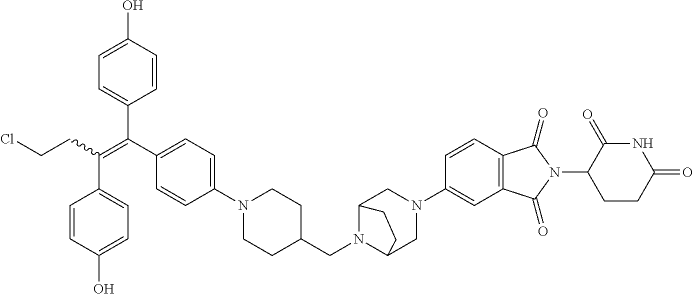 [SMILES: O=C1CCC(N2C(=O)c3ccc(N4CC5CCC(C4)N5CC4CCN(c5ccc(C(=C(CCCl)c6ccc(O)cc6)c6ccc(O)cc6)cc5)CC4)cc3C2=O)C(=O)N1] | 5-(8-((1-(4-(4-chloro-1,2-bis(4- hydroxyphenyl)but-1-en-1-yl)phenyl)piperidin- 4-yl)methyl)-3,8-diazabicyclo[3.2.1]octan-3- yl)-2-(2,6-dioxopiperidin-3-yl)isoindoline-1,3- dione |
| | 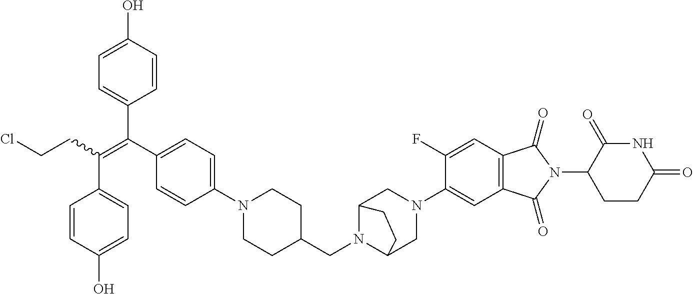 [SMILES: O=C1CCC(N2C(=O)c3cc(F)c(N4CC5CCC(C4)N5CC4CCN(c5ccc(C(=C(CCCl)c6ccc(O)cc6)c6ccc(O)cc6)cc5)CC4)cc3C2=O)C(=O)N1] | 5-(8-((1-(4-(4-chloro-1,2-bis(4- hydroxyphenyl)but-1-en-1-yl)phenyl)piperidin- 4-yl)methyl)-3,8-diazabicyclo[3.2.1]octan-3- yl)-2-(2,6-dioxopiperidin-3-yl)-6- fluoroisoindoline-1,3-dione |
| | 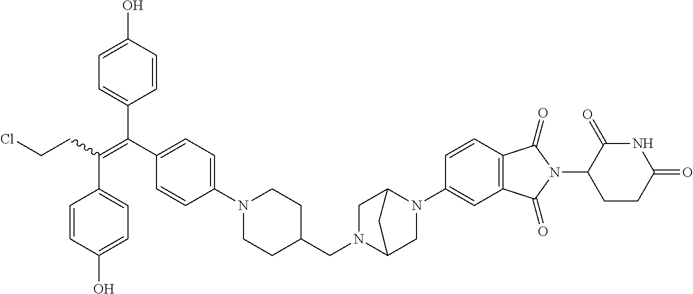 [SMILES: O=C1CCC(N2C(=O)c3ccc(N4CC5CC4CN5CC4CCN(c5ccc(C(=C(CCCl)c6ccc(O)cc6)c6ccc(O)cc6)cc5)CC4)cc3C2=O)C(=O)N1] | 5-(5-((1-(4-(4-chloro-1,2-bis(4- hydroxyphenyl)but-1-en-1-yl)phenyl)piperidin- 4-yl)methyl)-2,5-diazabicyclo[2.2.1]heptan-2- yl)-2-(2,6-dioxopiperidin-3-yl)isoindoline-1,3- dione |
| | 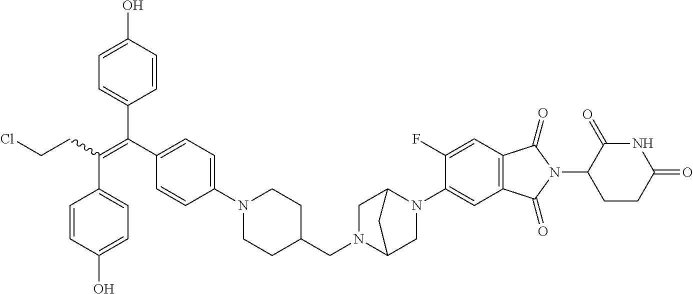 [SMILES: O=C1CCC(N2C(=O)c3cc(F)c(N4CC5CC4CN5CC4CCN(c5ccc(C(=C(CCCl)c6ccc(O)cc6)c6ccc(O)cc6)cc5)CC4)cc3C2=O)C(=O)N1] | 5-(5-((1-(4-(4-chloro-1,2-bis(4- hydroxyphenyl)but-1-en-1-yl)phenyl)piperidin- 4-yl)methyl)-2,5-diazabicyclo[2.2.1]heptan-2- yl)-2-(2,6-dioxopiperidin-3-yl)-6- fluoroisoindoline-1,3-dione |
| | 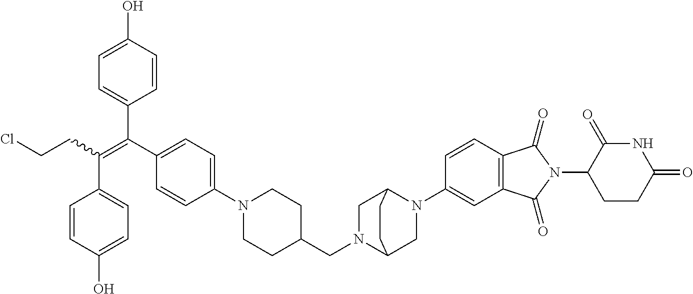 [SMILES: O=C1CCC(N2C(=O)c3ccc(N4CC5CCC4CN5CC4CCN(c5ccc(C(=C(CCCl)c6ccc(O)cc6)c6ccc(O)cc6)cc5)CC4)cc3C2=O)C(=O)N1] | 5-(5-((1-(4-(4-chloro-1,2-bis(4- hydroxyphenyl)but-1-en-1-yl)phenyl)piperidin- 4-yl)methyl)-2,5-diazabicyclo[2.2.2]octan-2- yl)-2-(2,6-dioxopiperidin-3-yl)isoindoline-1,3- dione |
| | 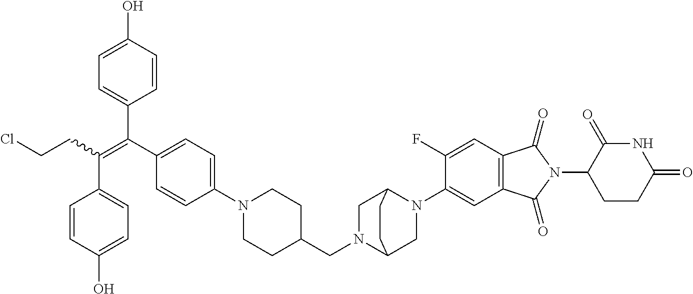 [SMILES: O=C1CCC(N2C(=O)c3cc(F)c(N4CC5CCC4CN5CC4CCN(c5ccc(C(=C(CCCl)c6ccc(O)cc6)c6ccc(O)cc6)cc5)CC4)cc3C2=O)C(=O)N1] | 5-(5-((1-(4-(4-chloro-1,2-bis(4- hydroxyphenyl)but-1-en-1-yl)phenyl)piperidin- 4-yl)methyl)-2,5-diazabicyclo[2.2.2]octan-2- yl)-2-(2,6-dioxopiperidin-3-yl)-6- fluoroisoindoline-1,3-dione |
| | 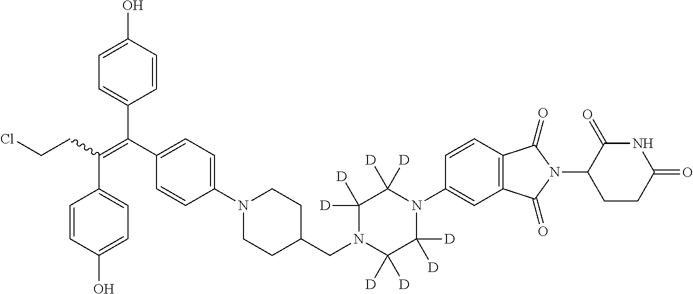 [SMILES: [2H]C1([2H])N(CC2CCN(c3ccc(C(=C(CCCl)c4ccc(O)cc4)c4ccc(O)cc4)cc3)CC2)C([2H])([2H])C([2H])([2H])N(c2ccc3c(c2)C(=O)N(C2CCC(=O)NC2=O)C3=O)C1([2H])[2H]] | 5-(4-((1-(4-(4-chloro-1,2-bis(4- hydroxyphenyl)but-1-en-1-yl)phenyl)piperidin- 4-yl)methyl)piperazin-1-yl-2,2,3,3,5,5,6,6-d₈)- 2-(2,6-dioxopiperidin-3-yl)isoindoline-1,3- dione |
| | 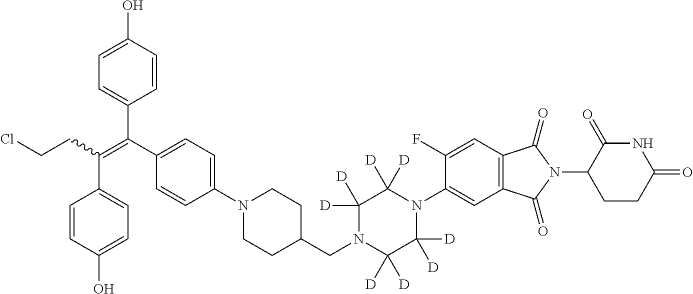 [SMILES: [2H]C1([2H])N(CC2CCN(c3ccc(C(=C(CCCl)c4ccc(O)cc4)c4ccc(O)cc4)cc3)CC2)C([2H])([2H])C([2H])([2H])N(c2cc3c(cc2F)C(=O)N(C2CCC(=O)NC2=O)C3=O)C1([2H])[2H]] | 5-(4-((1-(4-(4-chloro-1,2-bis(4- hydroxyphenyl)but-1-en-1-yl)phenyl)piperidin- 4-yl)methyl)piperazin-1-yl-2,2,3,3,5,5,6,6-d₈)- 2-(2,6-dioxopiperidin-3-yl)-6- fluoroisoindoline-1,3-dione |
| | 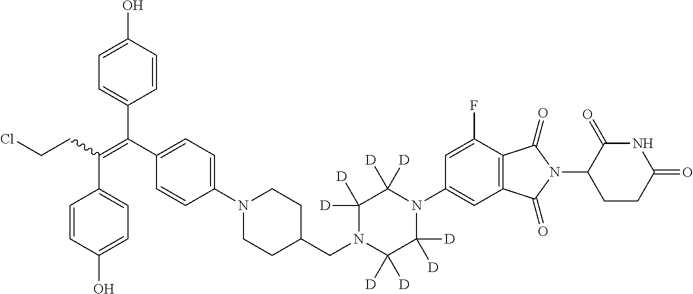 [SMILES: [2H]C1([2H])N(CC2CCN(c3ccc(C(=C(CCCl)c4ccc(O)cc4)c4ccc(O)cc4)cc3)CC2)C([2H])([2H])C([2H])([2H])N(c2cc(F)c3c(c2)C(=O)N(C2CCC(=O)NC2=O)C3=O)C1([2H])[2H]] | 6-(4-((1-(4-(4-chloro-1,2-bis(4- hydroxyphenyl)but-1-en-1-yl)phenyl)piperidin- 4-yl)methyl)piperazin-1-yl-2,2,3,3,5,5,6,6-d₈)- 2-(2,6-dioxopiperidin-3-yl)-4- fluoroisoindoline-1,3-dione |
| | 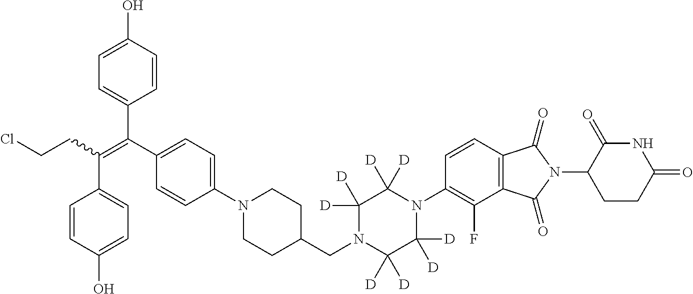 [SMILES: [2H]C1([2H])N(CC2CCN(c3ccc(C(=C(CCCl)c4ccc(O)cc4)c4ccc(O)cc4)cc3)CC2)C([2H])([2H])C([2H])([2H])N(c2ccc3c(c2F)C(=O)N(C2CCC(=O)NC2=O)C3=O)C1([2H])[2H]] | 5-(4-((1-(4-(4-chloro-1,2-bis(4- hydroxyphenyl)but-1-en-1-yl)phenyl)piperidin- 4-yl)methyl)piperazin-1-yl-2,2,3,3,5,5,6,6-d₈)- 2-(2,6-dioxopiperidin-3-yl)-4- fluoroisoindoline-1,3-dione |
| | 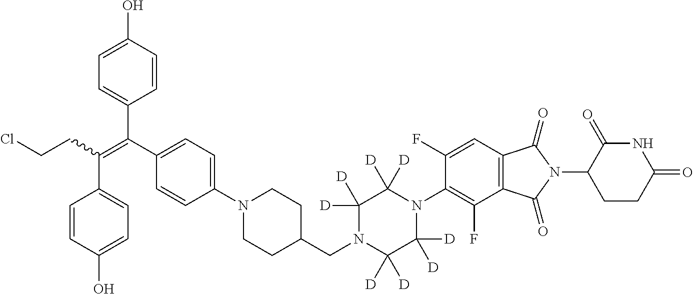 [SMILES: [2H]C1([2H])N(CC2CCN(c3ccc(C(=C(CCCl)c4ccc(O)cc4)c4ccc(O)cc4)cc3)CC2)C([2H])([2H])C([2H])([2H])N(c2c(F)cc3c(c2F)C(=O)N(C2CCC(=O)NC2=O)C3=O)C1([2H])[2H]] | 5-(4-((1-(4-(4-chloro-1,2-bis(4- hydroxyphenyl)but-1-en-1-yl)phenyl)piperidin- 4-yl)methyl)piperazin-1-yl-2,2,3,3,5,5,6,6-d₈)- 2-(2,6-dioxopiperidin-3-yl)-4,6- difluoroisoindoline-1,3-dione |
| | 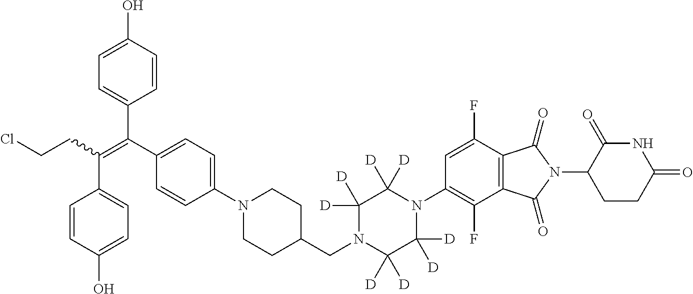 [SMILES: [2H]C1([2H])N(CC2CCN(c3ccc(C(=C(CCCl)c4ccc(O)cc4)c4ccc(O)cc4)cc3)CC2)C([2H])([2H])C([2H])([2H])N(c2cc(F)c3c(c2F)C(=O)N(C2CCC(=O)NC2=O)C3=O)C1([2H])[2H]] | 5-(4-((1-(4-(4-chloro-1,2-bis(4- hydroxyphenyl)but-1-en-1-yl)phenyl)piperidin- 4-yl)methyl)piperazin-1-yl-2,2,3,3,5,5,6,6-d₈)- 2-(2,6-dioxopiperidin-3-yl)-4,7- difluoroisoindoline-1,3-dione |
| | 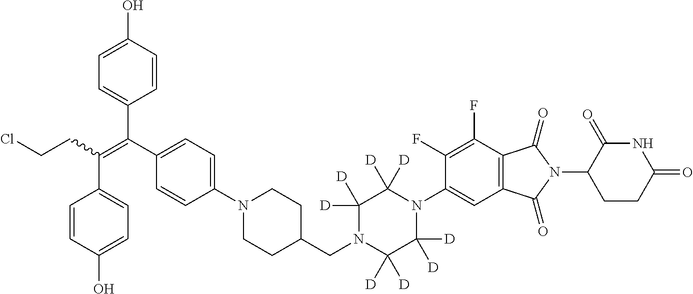 [SMILES: [2H]C1([2H])N(CC2CCN(c3ccc(C(=C(CCCl)c4ccc(O)cc4)c4ccc(O)cc4)cc3)CC2)C([2H])([2H])C([2H])([2H])N(c2cc3c(c(F)c2F)C(=O)N(C2CCC(=O)NC2=O)C3=O)C1([2H])[2H]] | 6-(4-((1-(4-(4-chloro-1,2-bis(4- hydroxyphenyl)but-1-en-1-yl)phenyl)piperidin- 4-yl)methyl)piperazin-1-yl-2,2,3,3,5,5,6,6-d₈)- 2-(2,6-dioxopiperidin-3-yl)-4,5- difluoroisoindoline-1,3-dione |
| | 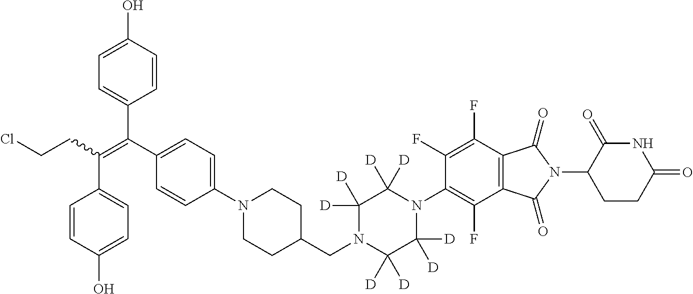 [SMILES: [2H]C1([2H])N(CC2CCN(c3ccc(C(=C(CCCl)c4ccc(O)cc4)c4ccc(O)cc4)cc3)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(4-(4-chloro-1,2-bis(4- hydroxyphenyl)but-1-en-1-yl)phenyl)piperidin- 4-yl)methyl)piperazin-1-yl-2,2,3,3,5,5,6,6-d₈)- 2-(2,6-dioxopiperidin-3-yl)-4,6,7- trifluoroisoindoline-1,3-dione |
| | 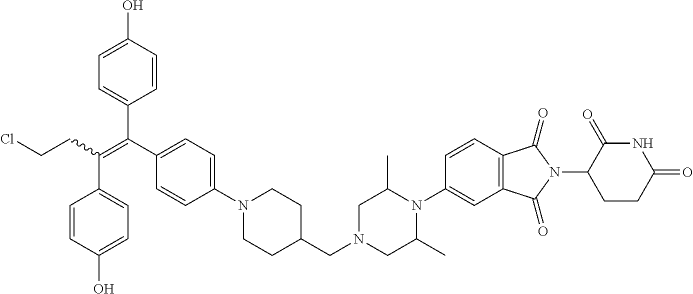 [SMILES: CC1CN(CC2CCN(c3ccc(C(=C(CCCl)c4ccc(O)cc4)c4ccc(O)cc4)cc3)CC2)CC(C)N1c1ccc2c(c1)C(=O)N(C1CCC(=O)NC1=O)C2=O] | 5-(4-((1-(4-(4-chloro-1,2-bis(4- hydroxyphenyl)but-1-en-1-yl)phenyl)piperidin- 4-yl)methyl)-2,6-dimethylpiperazin-1-yl)-2- (2,6-dioxopiperidin-3-yl)isoindoline-1,3-dione |
| | 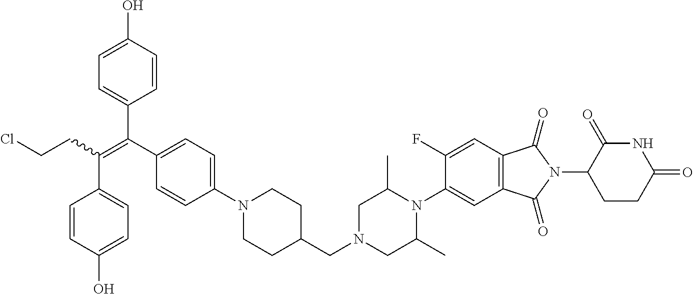 [SMILES: CC1CN(CC2CCN(c3ccc(C(=C(CCCl)c4ccc(O)cc4)c4ccc(O)cc4)cc3)CC2)CC(C)N1c1cc2c(cc1F)C(=O)N(C1CCC(=O)NC1=O)C2=O] | 5-(4-((1-(4-(4-chloro-1,2-bis(4- hydroxyphenyl)but-1-en-1-yl)phenyl)piperidin- 4-yl)methyl)-2,6-dimethylpiperazin-1-yl)-2- (2,6-dioxopiperidin-3-yl)-6-fluoroisoindoline- 1,3-dione |
| | 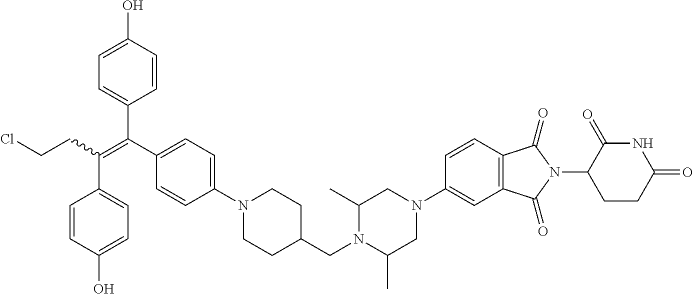 [SMILES: CC1CN(c2ccc3c(c2)C(=O)N(C2CCC(=O)NC2=O)C3=O)CC(C)N1CC1CCN(c2ccc(C(=C(CCCl)c3ccc(O)cc3)c3ccc(O)cc3)cc2)CC1] | 5-(4-((1-(4-(4-chloro-1,2-bis(4- hydroxyphenyl)but-1-en-1-yl)phenyl)piperidin- 4-yl)methyl)-3,5-dimethylpiperazin-1-yl)-2- (2,6-dioxopiperidin-3-yl)isoindoline-1,3-dione |
| | 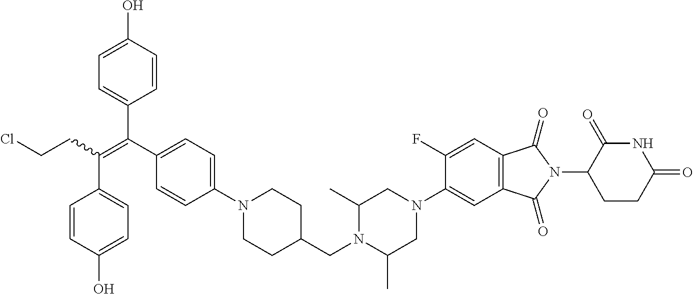 [SMILES: CC1CN(c2cc3c(cc2F)C(=O)N(C2CCC(=O)NC2=O)C3=O)CC(C)N1CC1CCN(c2ccc(C(=C(CCCl)c3ccc(O)cc3)c3ccc(O)cc3)cc2)CC1] | 5-(4-((1-(4-(4-chloro-1,2-bis(4- hydroxyphenyl)but-1-en-1-yl)phenyl)piperidin- 4-yl)methyl)-3,5-dimethylpiperazin-1-yl)-2- (2,6-dioxopiperidin-3-yl)-6-fluoroisoindoline- 1,3-dione |
| | 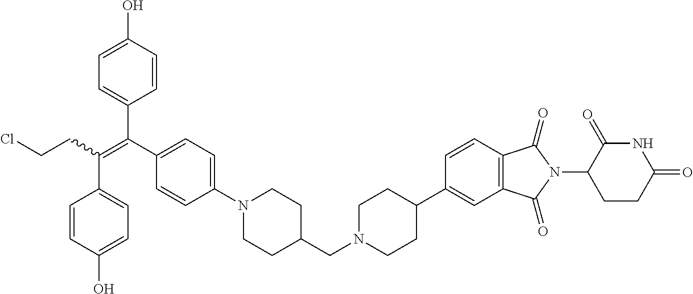 [SMILES: O=C1CCC(N2C(=O)c3ccc(C4CCN(CC5CCN(c6ccc(C(=C(CCCl)c7ccc(O)cc7)c7ccc(O)cc7)cc6)CC5)CC4)cc3C2=O)C(=O)N1] | 5-(1-((1-(4-(4-chloro-1,2-bis(4- hydroxyphenyl)but-1-en-1-yl)phenyl)piperidin- 4-yl)methyl)piperidin-4-yl)-2-(2,6- dioxopiperidin-3-yl)isoindoline-1,3-dione |
| | 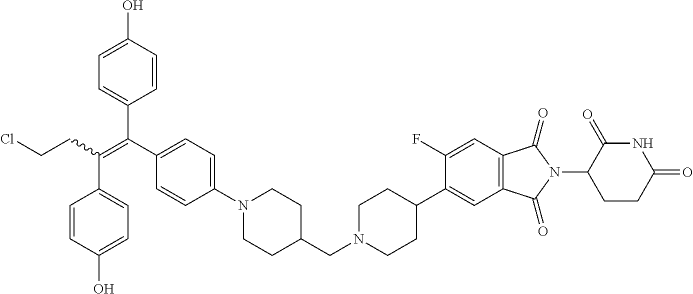 [SMILES: O=C1CCC(N2C(=O)c3cc(F)c(C4CCN(CC5CCN(c6ccc(C(=C(CCCl)c7ccc(O)cc7)c7ccc(O)cc7)cc6)CC5)CC4)cc3C2=O)C(=O)N1] | 5-(1-((1-(4-(4-chloro-1,2-bis(4- hydroxyphenyl)but-1-en-1-yl)phenyl)piperidin- 4-yl)methyl)piperidin-4-yl)-2-(2,6- dioxopiperidin-3-yl)-6-fluoroisoindoline-1,3- dione |
| | 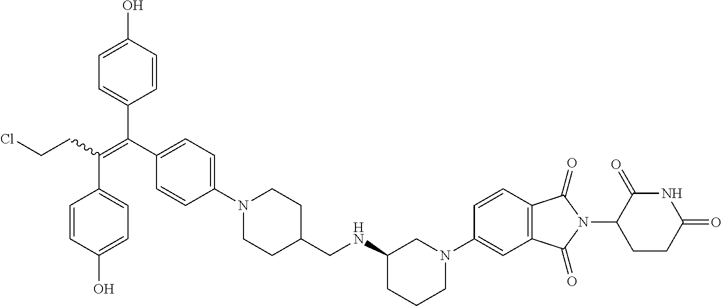 [SMILES: O=C1CCC(N2C(=O)c3ccc(N4CCC[C@@H](NCC5CCN(c6ccc(C(=C(CCCl)c7ccc(O)cc7)c7ccc(O)cc7)cc6)CC5)C4)cc3C2=O)C(=O)N1] | 5-((R)-3-(((1-(4-(4-chloro-1,2-bis(4- hydroxyphenyl)but-1-en-1-yl)phenyl)piperidin- 4-yl)methyl)amino)piperidin-1-yl)-2-(2,6- dioxopiperidin-3-yl)isoindoline-1,3-dione |
| | 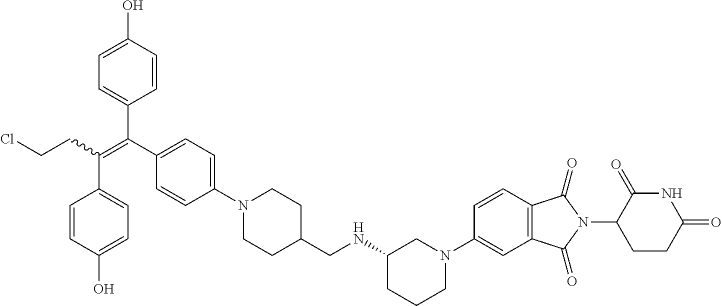 [SMILES: O=C1CCC(N2C(=O)c3ccc(N4CCC[C@H](NCC5CCN(c6ccc(C(=C(CCCl)c7ccc(O)cc7)c7ccc(O)cc7)cc6)CC5)C4)cc3C2=O)C(=O)N1] | 5-((S)-3-(((1-(4-(4-chloro-1,2-bis(4- hydroxyphenyl)but-1-en-1-yl)phenyl)piperidin- 4-yl)methyl)amino)piperidin-1-yl)-2-(2,6- dioxopiperidin-3-yl)isoindoline-1,3-dione |
| | 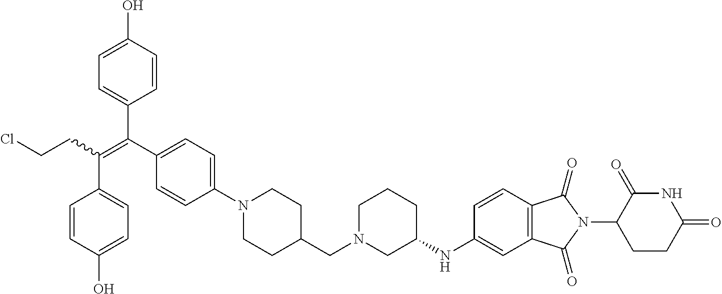 [SMILES: O=C1CCC(N2C(=O)c3ccc(N[C@H]4CCCN(CC5CCN(c6ccc(C(=C(CCCl)c7ccc(O)cc7)c7ccc(O)cc7)cc6)CC5)C4)cc3C2=O)C(=O)N1] | 5-(((S)-1-((1-(4-(4-chloro-1,2-bis(4- hydroxyphenyl)but-1-en-1-yl)phenyl)piperidin- 4-yl)methyl)piperidin-3-yl)amino)-2-(2,6- dioxopiperidin-3-yl)isoindoline-1,3-dione |
| | 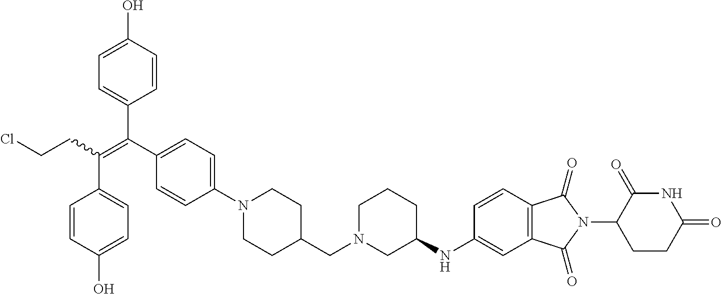 [SMILES: O=C1CCC(N2C(=O)c3ccc(N[C@@H]4CCCN(CC5CCN(c6ccc(C(=C(CCCl)c7ccc(O)cc7)c7ccc(O)cc7)cc6)CC5)C4)cc3C2=O)C(=O)N1] | 5-(((R)-1-((1-(4-(4-chloro-1,2-bis(4- hydroxyphenyl)but-1-en-1-yl)phenyl)piperidin- 4-yl)methyl)piperidin-3-yl)amino)-2-(2,6- dioxopiperidin-3-yl)isoindoline-1,3-dione |
| | 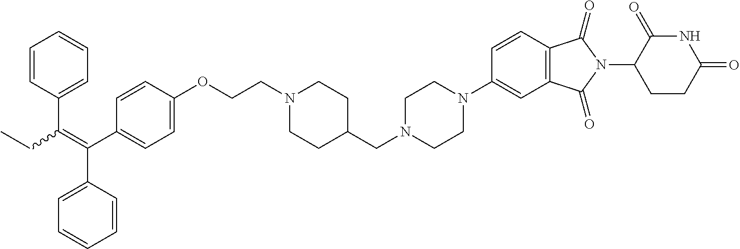 [SMILES: CCC(=C(c1ccccc1)c1ccc(OCCN2CCC(CN3CCN(c4ccc5c(c4)C(=O)N(C4CCC(=O)NC4=O)C5=O)CC3)CC2)cc1)c1ccccc1] | 2-(2,6-dioxopiperidin-3-yl)-5-(4-((1-(2-(4-(1,2- diphenylbut-1-en-1-yl)phenoxy)ethyl)piperidin- 4-yl)methyl)piperazin-1-yl)isoindoline-1,3- dione |
| | 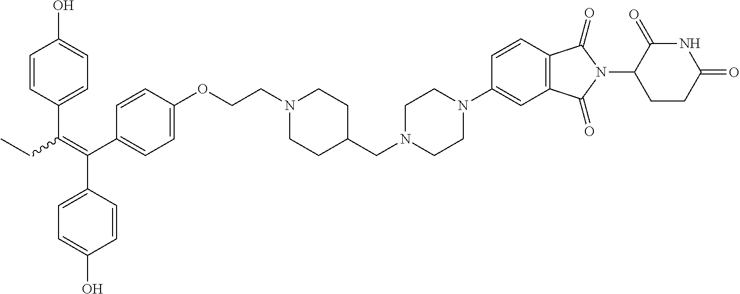 [SMILES: CCC(=C(c1ccc(O)cc1)c1ccc(OCCN2CCC(CN3CCN(c4ccc5c(c4)C(=O)N(C4CCC(=O)NC4=O)C5=O)CC3)CC2)cc1)c1ccc(O)cc1] | 5-(4-((1-(2-(4-(1,2-bis(4-hydroxyphenyl)but-1- en-1-yl)phenoxy)ethyl)piperidin-4- yl)methyl)piperazin-1-yl)-2-(2,6- dioxopiperidin-3-yl)isoindoline-1,3-dione |
| | 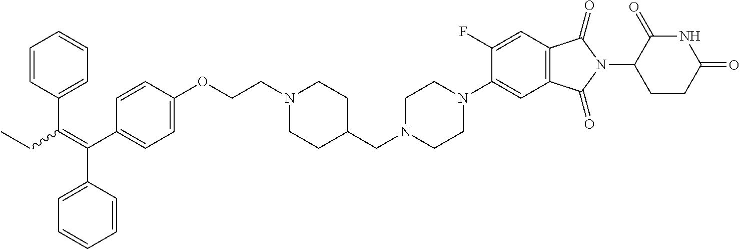 [SMILES: CCC(=C(c1ccccc1)c1ccc(OCCN2CCC(CN3CCN(c4cc5c(cc4F)C(=O)N(C4CCC(=O)NC4=O)C5=O)CC3)CC2)cc1)c1ccccc1] | 2-(2,6-dioxopiperidin-3-yl)-5-(4-((1-(2-(4-(1,2- diphenylbut-1-en-1-yl)phenoxy)ethyl)piperidin- 4-yl)methyl)piperazin-1-yl)-6- fluoroisoindoline-1,3-dione |
| | 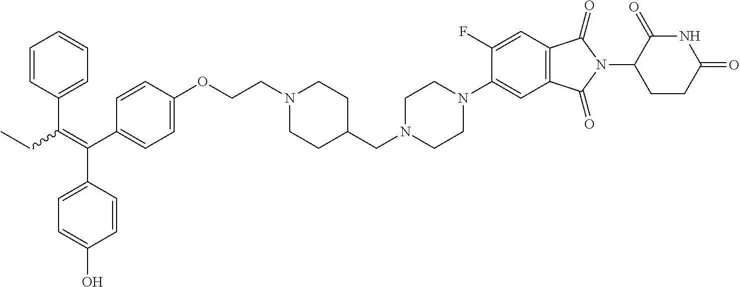 [SMILES: CCC(=C(c1ccc(O)cc1)c1ccc(OCCN2CCC(CN3CCN(c4cc5c(cc4F)C(=O)N(C4CCC(=O)NC4=O)C5=O)CC3)CC2)cc1)c1ccccc1] | 2-(2,6-dioxopiperidin-3-yl)-5-fluoro-6-(4-((1- (2-(4-(1-(4-hydroxyphenyl)-2-phenylbut-1-en- 1-yl)phenoxy)ethyl)piperidin-4- yl)methyl)piperazin-1-yl)isoindoline-1,3-dione |
| | 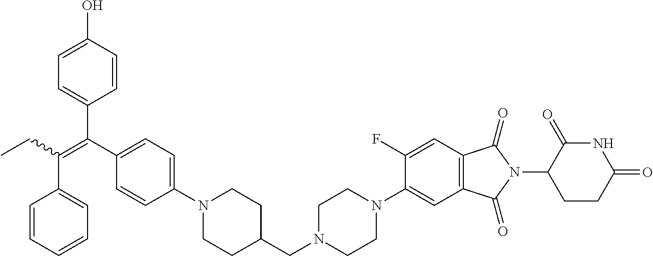 [SMILES: CCC(=C(c1ccc(O)cc1)c1ccc(N2CCC(CN3CCN(c4cc5c(cc4F)C(=O)N(C4CCC(=O)NC4=O)C5=O)CC3)CC2)cc1)c1ccccc1] | 2-(2,6-dioxopiperidin-3-yl)-5-fluoro-6-(4-((1- (4-(1-(4-hydroxyphenyl)-2-phenylbut-1-en-1- yl)phenyl)piperidin-4-yl)methyl)piperazin-1- yl)isoindoline-1,3-dione |
| | 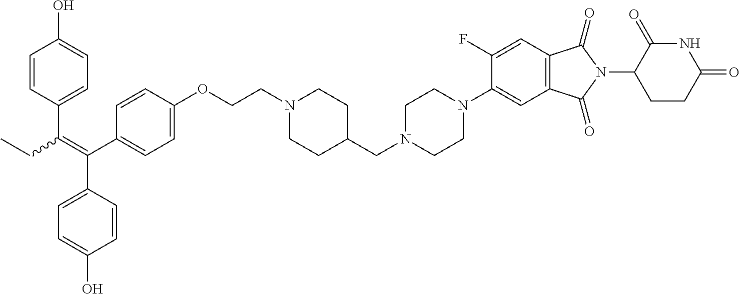 [SMILES: CCC(=C(c1ccc(O)cc1)c1ccc(OCCN2CCC(CN3CCN(c4cc5c(cc4F)C(=O)N(C4CCC(=O)NC4=O)C5=O)CC3)CC2)cc1)c1ccc(O)cc1] | 5-(4-((1-(2-(4-(1,2-bis(4-hydroxyphenyl)but-1- en-1-yl)phenoxy)ethyl)piperidin-4- yl)methyl)piperazin-1-yl)-2-(2,6- dioxopiperidin-3-yl)-6-fluoroisoindoline-1,3- dione |
| | 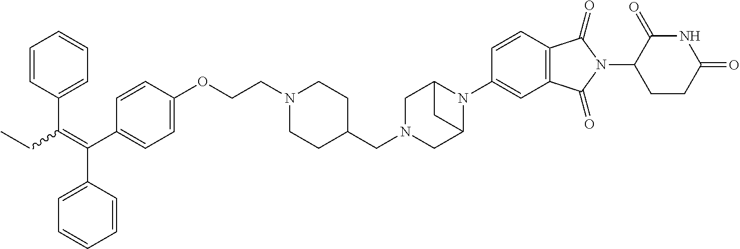 [SMILES: CCC(=C(c1ccccc1)c1ccc(OCCN2CCC(CN3CC4CC(C3)N4c3ccc4c(c3)C(=O)N(C3CCC(=O)NC3=O)C4=O)CC2)cc1)c1ccccc1] | 2-(2,6-dioxopiperidin-3-yl)-5-(3-((1-(2-(4-(1,2- diphenylbut-1-en-1-yl)phenoxy)ethyl)piperidin- 4-yl)methyl)-3,6-diazabicyclo[3.1.1]heptan-6- yl)isoindoline-1,3-dione |
| | 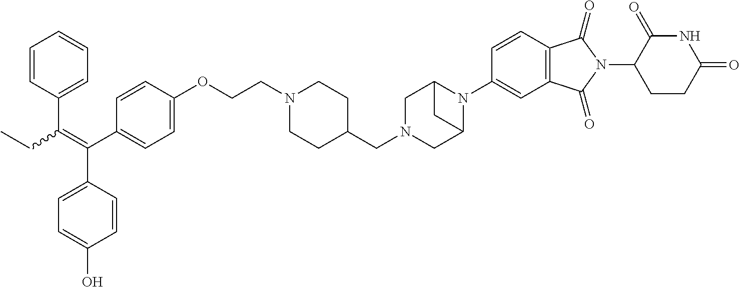 [SMILES: CCC(=C(c1ccc(O)cc1)c1ccc(OCCN2CCC(CN3CC4CC(C3)N4c3ccc4c(c3)C(=O)N(C3CCC(=O)NC3=O)C4=O)CC2)cc1)c1ccccc1] | 2-(2,6-dioxopiperidin-3-yl)-5-(3-((1-(2-(4-(1- (4-hydroxyphenyl)-2-phenylbut-1-en-1- yl)phenoxy)ethyl)piperidin-4-yl)methyl)-3,6- diazabicyclo[3.1.1]heptan-6-yl)isoindoline-1,3- dione |
| | 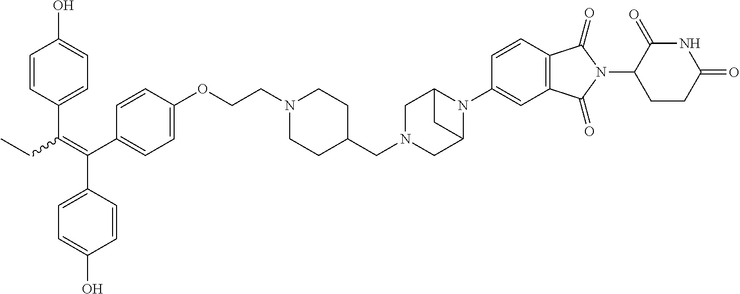 [SMILES: CCC(=C(c1ccc(O)cc1)c1ccc(OCCN2CCC(CN3CC4CC(C3)N4c3ccc4c(c3)C(=O)N(C3CCC(=O)NC3=O)C4=O)CC2)cc1)c1ccc(O)cc1] | 5-(3-((1-(2-(4-(1,2-bis(4-hydroxyphenyl)but-1- en-1-yl)phenoxy)ethyl)piperidin-4-yl)methyl)- 3,6-diazabicyclo[3.1.1]heptan-6-yl)-2-(2,6- dioxopiperidin-3-yl)isoindoline-1,3-dione |
| | 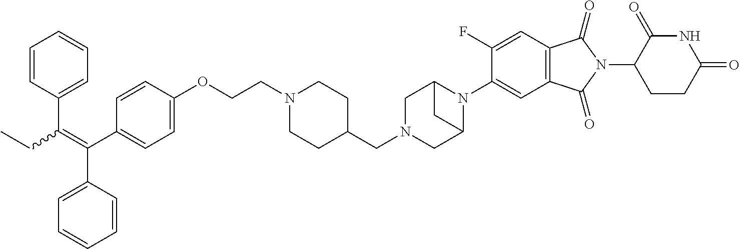 [SMILES: CCC(=C(c1ccccc1)c1ccc(OCCN2CCC(CN3CC4CC(C3)N4c3cc4c(cc3F)C(=O)N(C3CCC(=O)NC3=O)C4=O)CC2)cc1)c1ccccc1] | 2-(2,6-dioxopiperidin-3-yl)-5-(3-((1-(2-(4-(1,2- diphenylbut-1-en-1-yl)phenoxy)ethyl)piperidin- 4-yl)methyl)-3,6-diazabicyclo[3.1.1]heptan-6- yl)-6-fluoroisoindoline-1,3-dione |
| | 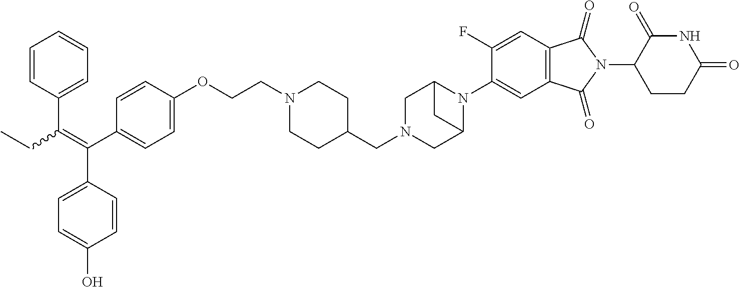 [SMILES: CCC(=C(c1ccc(O)cc1)c1ccc(OCCN2CCC(CN3CC4CC(C3)N4c3cc4c(cc3F)C(=O)N(C3CCC(=O)NC3=O)C4=O)CC2)cc1)c1ccccc1] | 2-(2,6-dioxopiperidin-3-yl)-5-fluoro-6-(3-((1- (2-(4-(1-(4-hydroxyphenyl)-2-phenylbut-1-en- 1-yl)phenoxy)ethyl)piperidin-4-yl)methyl)-3,6- diazabicyclo[3.1.1]heptan-6-yl)isoindoline-1,3- dione |
| | 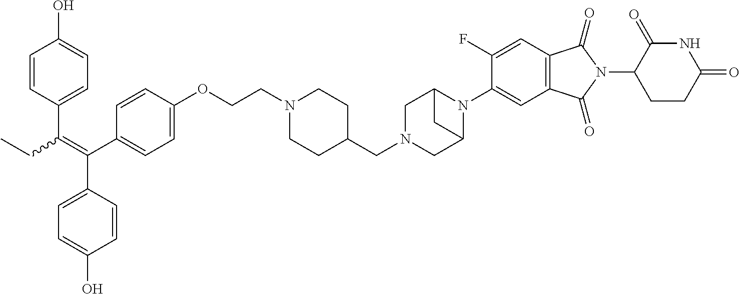 [SMILES: CCC(=C(c1ccc(O)cc1)c1ccc(OCCN2CCC(CN3CC4CC(C3)N4c3cc4c(cc3F)C(=O)N(C3CCC(=O)NC3=O)C4=O)CC2)cc1)c1ccc(O)cc1] | 5-(3-((1-(2-(4-(1,2-bis(4-hydroxyphenyl)but-1- en-1-yl)phenoxy)ethyl)piperidin-4-yl)methyl)- 3,6-diazabicyclo[3.1.1]heptan-6-yl)-2-(2,6- dioxopiperidin-3-yl)-6-fluoroisoindoline-1,3- dione |
| | 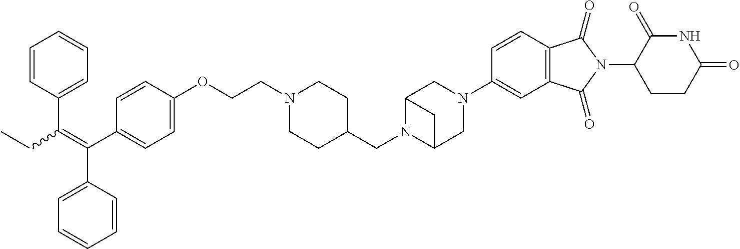 [SMILES: CCC(=C(c1ccccc1)c1ccc(OCCN2CCC(CN3C4CC3CN(c3ccc5c(c3)C(=O)N(C3CCC(=O)NC3=O)C5=O)C4)CC2)cc1)c1ccccc1] | 2-(2,6-dioxopiperidin-3-yl)-5-(6-((1-(2-(4-(1,2- diphenylbut-1-en-1-yl)phenoxy)ethyl)piperidin- 4-yl)methyl)-3,6-diazabicyclo[3.1.1]heptan-3- yl)isoindoline-1,3-dione |
| | 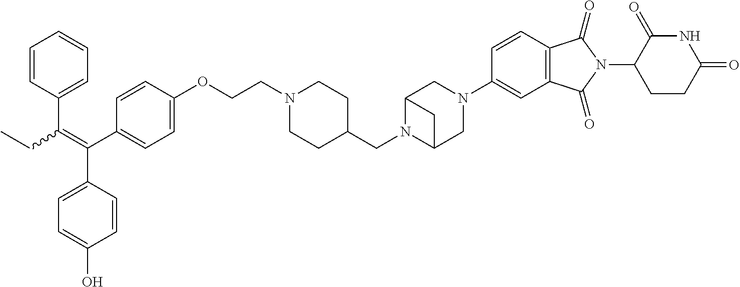 [SMILES: CCC(=C(c1ccc(O)cc1)c1ccc(OCCN2CCC(CN3C4CC3CN(c3ccc5c(c3)C(=O)N(C3CCC(=O)NC3=O)C5=O)C4)CC2)cc1)c1ccccc1] | 2-(2,6-dioxopiperidin-3-yl)-5-(6-((1-(2-(4-(1- (4-hydroxyphenyl)-2-phenylbut-1-en-1- yl)phenoxy)ethyl)piperidin-4-yl)methyl)-3,6- diazabicyclo[3.1.1]heptan-3-yl)isoindoline-1,3- dione |
| | 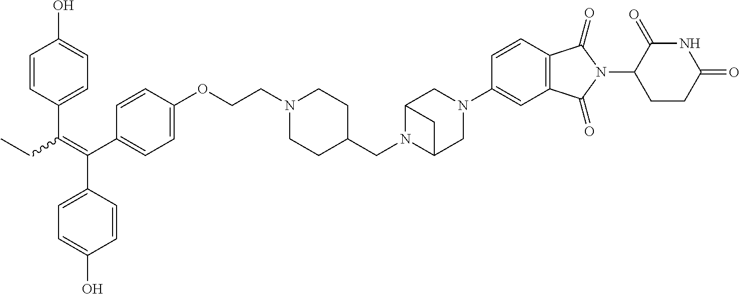 [SMILES: CCC(=C(c1ccc(O)cc1)c1ccc(OCCN2CCC(CN3C4CC3CN(c3ccc5c(c3)C(=O)N(C3CCC(=O)NC3=O)C5=O)C4)CC2)cc1)c1ccc(O)cc1] | 5-(6-((1-(2-(4-(1,2-bis(4-hydroxyphenyl)but-1- en-1-yl)phenoxy)ethyl)piperidin-4-yl)methyl)- 3,6-diazabicyclo[3.1.1]heptan-3-yl)-2-(2,6- dioxopiperidin-3-yl)isoindoline-1,3-dione |
| | 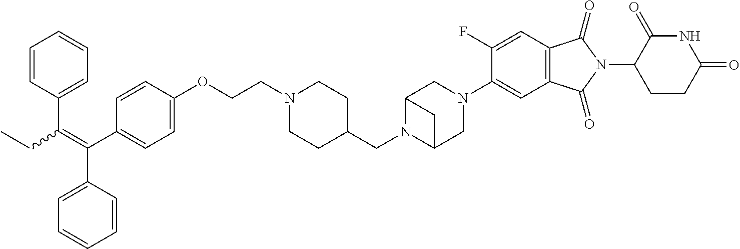 [SMILES: CCC(=C(c1ccccc1)c1ccc(OCCN2CCC(CN3C4CC3CN(c3cc5c(cc3F)C(=O)N(C3CCC(=O)NC3=O)C5=O)C4)CC2)cc1)c1ccccc1] | 2-(2,6-dioxopiperidin-3-yl)-5-(6-((1-(2-(4-(1,2- diphenylbut-1-en-1-yl)phenoxy)ethyl)piperidin- 4-yl)methyl)-3,6-diazabicyclo[3.1.1]heptan-3- yl)-6-fluoroisoindoline-1,3-dione |
| | 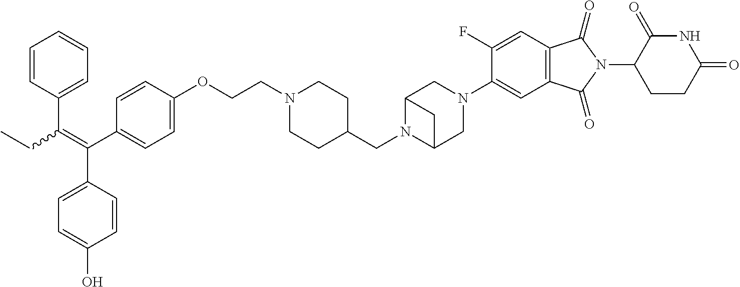 [SMILES: CCC(=C(c1ccc(O)cc1)c1ccc(OCCN2CCC(CN3C4CC3CN(c3cc5c(cc3F)C(=O)N(C3CCC(=O)NC3=O)C5=O)C4)CC2)cc1)c1ccccc1] | 2-(2,6-dioxopiperidin-3-yl)-5-fluoro-6-(6-((1- (2-(4-(1-(4-hydroxyphenyl)-2-phenylbut-1-en- 1-yl)phenoxy)ethyl)piperidin-4-yl)methyl)-3,6- diazabicyclo[3.1.1]heptan-3-yl)isoindoline-1,3- dione |
| | 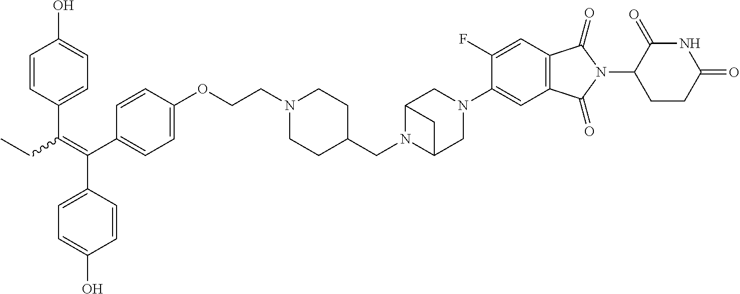 [SMILES: CCC(=C(c1ccc(O)cc1)c1ccc(OCCN2CCC(CN3C4CC3CN(c3cc5c(cc3F)C(=O)N(C3CCC(=O)NC3=O)C5=O)C4)CC2)cc1)c1ccc(O)cc1] | 5-(6-((1-(2-(4-(1,2-bis(4-hydroxyphenyl)but-1- en-1-yl)phenoxy)ethyl)piperidin-4-yl)methyl)- 3,6-diazabicyclo[3.1.1]heptan-3-yl)-2-(2,6- dioxopiperidin-3-yl)-6-fluoroisoindoline-1,3- dione |
| | 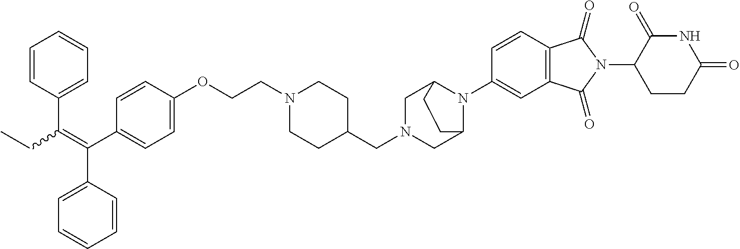 [SMILES: CCC(=C(c1ccccc1)c1ccc(OCCN2CCC(CN3CC4CCC(C3)N4c3ccc4c(c3)C(=O)N(C3CCC(=O)NC3=O)C4=O)CC2)cc1)c1ccccc1] | 2-(2,6-dioxopiperidin-3-yl)-5-(3-((1-(2-(4-(1,2- diphenylbut-1-en-1-yl)phenoxy)ethyl)piperidin- 4-yl)methyl)-3,8-diazabicyclo[3.2.1]octan-8- yl)isoindoline-1,3-dione |
| | 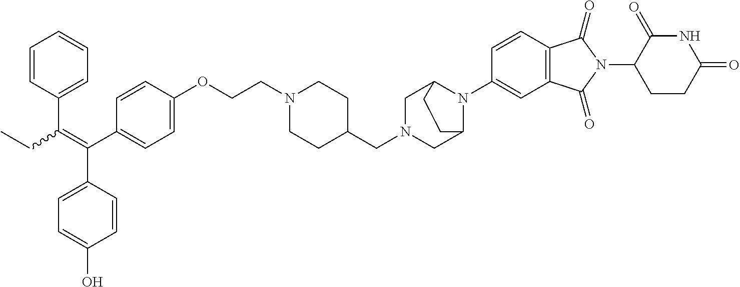 [SMILES: CCC(=C(c1ccc(O)cc1)c1ccc(OCCN2CCC(CN3CC4CCC(C3)N4c3ccc4c(c3)C(=O)N(C3CCC(=O)NC3=O)C4=O)CC2)cc1)c1ccccc1] | 2-(2,6-dioxopiperidin-3-yl)-5-(3-((1-(2-(4-(1- (4-hydroxyphenyl)-2-phenylbut-1-en-1- yl)phenoxy)ethyl)piperidin-4-yl)methyl)-3,8- diazabicyclo[3.2.1]octan-8-yl)isoindoline-1,3- dione |
| | 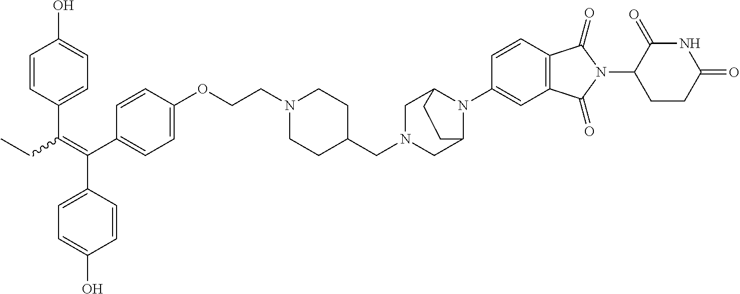 [SMILES: CCC(=C(c1ccc(O)cc1)c1ccc(OCCN2CCC(CN3CC4CCC(C3)N4c3ccc4c(c3)C(=O)N(C3CCC(=O)NC3=O)C4=O)CC2)cc1)c1ccc(O)cc1] | 5-(3-((1-(2-(4-(1,2-bis(4-hydroxyphenyl)but-1- en-1-yl)phenoxy)ethyl)piperidin-4-yl)methyl)- 3,8-diazabicyclo[3.2.1]octan-8-yl)-2-(2,6- dioxopiperidin-3-yl)isoindoline-1,3-dione |
| | 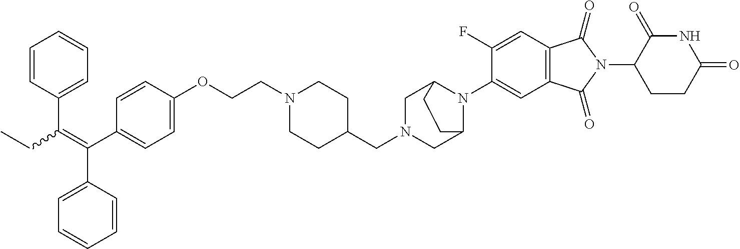 [SMILES: CCC(=C(c1ccccc1)c1ccc(OCCN2CCC(CN3CC4CCC(C3)N4c3cc4c(cc3F)C(=O)N(C3CCC(=O)NC3=O)C4=O)CC2)cc1)c1ccccc1] | 2-(2,6-dioxopiperidin-3-yl)-5-(3-((1-(2-(4-(1,2- diphenylbut-1-en-1-yl)phenoxy)ethyl)piperidin- 4-yl)methyl)-3,8-diazabicyclo[3.2.1]octan-8- yl)-6-fluoroisoindoline-1,3-dione |
| | 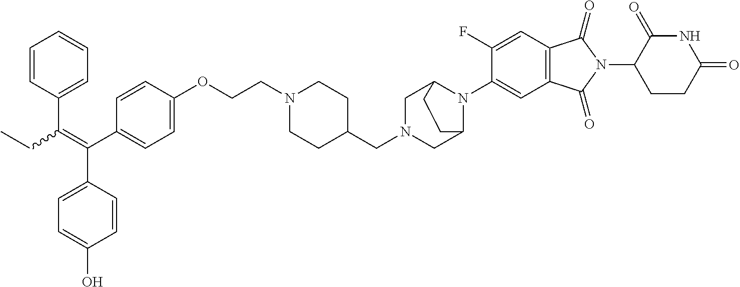 [SMILES: CCC(=C(c1ccc(O)cc1)c1ccc(OCCN2CCC(CN3CC4CCC(C3)N4c3cc4c(cc3F)C(=O)N(C3CCC(=O)NC3=O)C4=O)CC2)cc1)c1ccccc1] | 2-(2,6-dioxopiperidin-3-yl)-5-fluoro-6-(3-((1- (2-(4-(1-(4-hydroxyphenyl)-2-phenylbut-1-en- 1-yl)phenoxy)ethyl)piperidin-4-yl)methyl)-3,8- diazabicyclo[3.2.1]octan-8-yl)isoindoline-1,3- dione |
| | 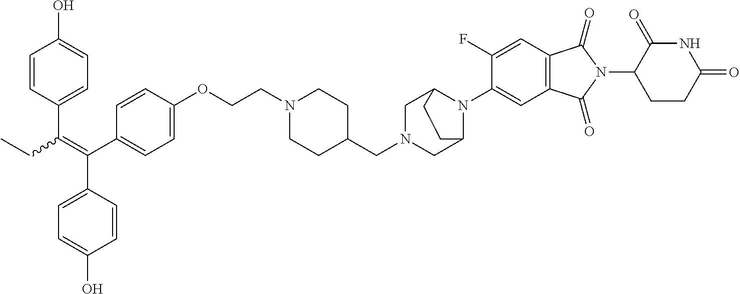 [SMILES: CCC(=C(c1ccc(O)cc1)c1ccc(OCCN2CCC(CN3CC4CCC(C3)N4c3cc4c(cc3F)C(=O)N(C3CCC(=O)NC3=O)C4=O)CC2)cc1)c1ccc(O)cc1] | 5-(3-((1-(2-(4-(1,2-bis(4-hydroxyphenyl)but-1- en-1-yl)phenoxy)ethyl)piperidin-4-yl)methyl)- 3,8-diazabicyclo[3.2.1]octan-8-yl)-2-(2,6- dioxopiperidin-3-yl)-6-fluoroisoindoline-1,3- dione |
| | 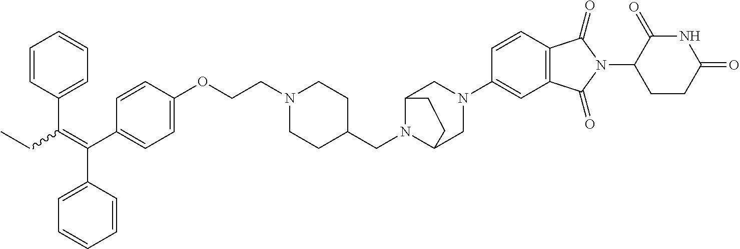 [SMILES: CCC(=C(c1ccccc1)c1ccc(OCCN2CCC(CN3C4CCC3CN(c3ccc5c(c3)C(=O)N(C3CCC(=O)NC3=O)C5=O)C4)CC2)cc1)c1ccccc1] | 2-(2,6-dioxopiperidin-3-yl)-5-(8-((1-(2-(4-(1,2- diphenylbut-1-en-1-yl)phenoxy)ethyl)piperidin- 4-yl)methyl)-3,8-diazabicyclo[3.2.1]octan-3- yl)isoindoline-1,3-dione |
| | 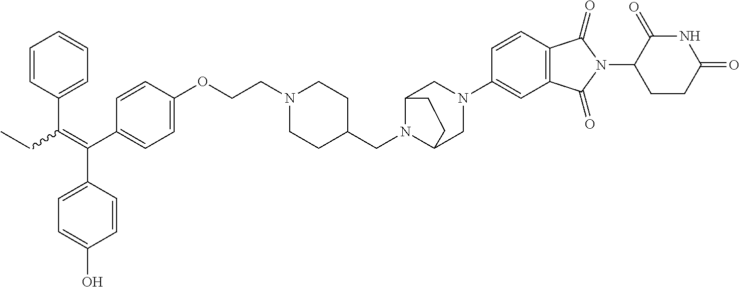 [SMILES: CCC(=C(c1ccc(O)cc1)c1ccc(OCCN2CCC(CN3C4CCC3CN(c3ccc5c(c3)C(=O)N(C3CCC(=O)NC3=O)C5=O)C4)CC2)cc1)c1ccccc1] | 2-(2,6-dioxopiperidin-3-yl)-5-(8-((1-(2-(4-(1- (4-hydroxyphenyl)-2-phenylbut-1-en-1- yl)phenoxy)ethyl)piperidin-4-yl)methyl)-3,8- diazabicyclo[3.2.1]octan-3-yl)isoindoline-1,3- dione |
| | 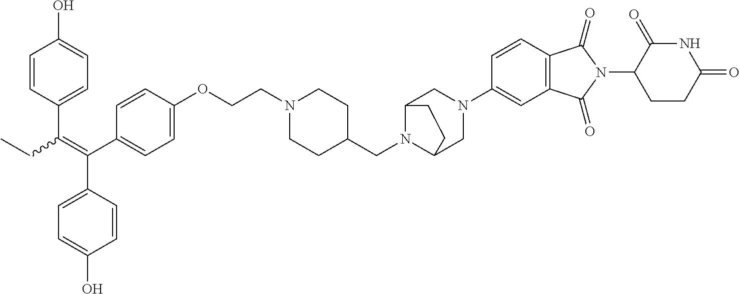 [SMILES: CCC(=C(c1ccc(O)cc1)c1ccc(OCCN2CCC(CN3C4CCC3CN(c3ccc5c(c3)C(=O)N(C3CCC(=O)NC3=O)C5=O)C4)CC2)cc1)c1ccc(O)cc1] | 5-(8-((1-(2-(4-(1,2-bis(4-hydroxyphenyl)but-1- en-1-yl)phenoxy)ethyl)piperidin-4-yl)methyl)- 3,8-diazabicyclo[3.2.1]octan-3-yl)-2-(2,6- dioxopiperidin-3-yl)isoindoline-1,3-dione |
| | 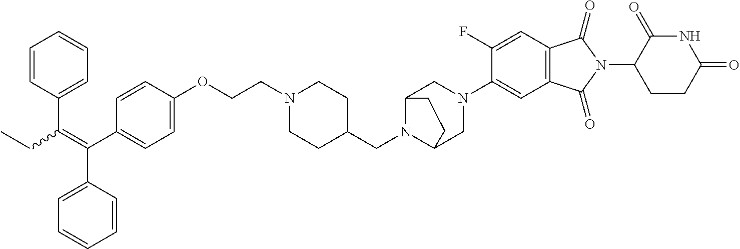 [SMILES: CCC(=C(c1ccccc1)c1ccc(OCCN2CCC(CN3C4CCC3CN(c3cc5c(cc3F)C(=O)N(C3CCC(=O)NC3=O)C5=O)C4)CC2)cc1)c1ccccc1] | 2-(2,6-dioxopiperidin-3-yl)-5-(8-((1-(2-(4-(1,2- diphenylbut-1-en-1-yl)phenoxy)ethyl)piperidin- 4-yl)methyl)-3,8-diazabicyclo[3.2.1]octan-3- yl)-6-fluoroisoindoline-1,3-dione |
| | 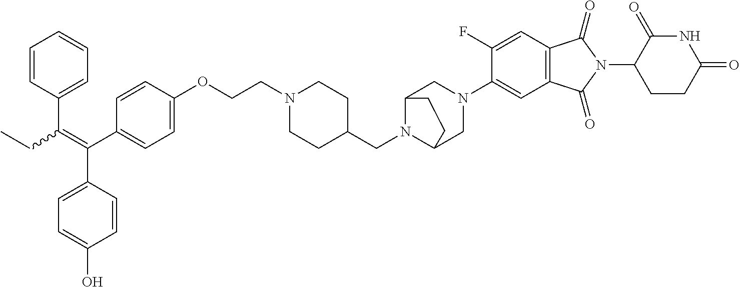 [SMILES: CCC(=C(c1ccc(O)cc1)c1ccc(OCCN2CCC(CN3C4CCC3CN(c3cc5c(cc3F)C(=O)N(C3CCC(=O)NC3=O)C5=O)C4)CC2)cc1)c1ccccc1] | 2-(2,6-dioxopiperidin-3-yl)-5-fluoro-6-(8-((1- (2-(4-(1-(4-hydroxyphenyl)-2-phenylbut-1-en- 1-yl)phenoxy)ethyl)piperidin-4-yl)methyl)-3,8- diazabicyclo[3.2.1]octan-3-yl)isoindoline-1,3- dione |
| | 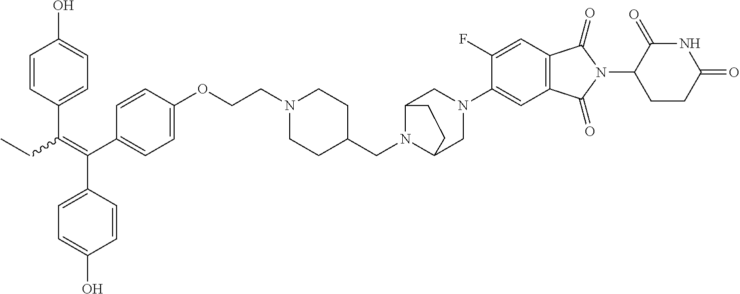 [SMILES: CCC(=C(c1ccc(O)cc1)c1ccc(OCCN2CCC(CN3C4CCC3CN(c3cc5c(cc3F)C(=O)N(C3CCC(=O)NC3=O)C5=O)C4)CC2)cc1)c1ccc(O)cc1] | 5-(8-((1-(2-(4-(1,2-bis(4-hydroxyphenyl)but-1- en-1-yl)phenoxy)ethyl)piperidin-4-yl)methyl)- 3,8-diazabicyclo[3.2.1]octan-3-yl)-2-(2,6- dioxopiperidin-3-yl)-6-fluoroisoindoline-1,3- dione |
| | 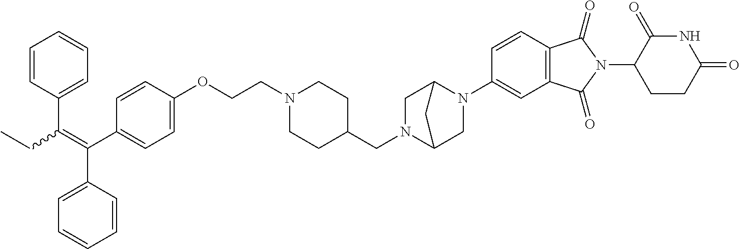 [SMILES: CCC(=C(c1ccccc1)c1ccc(OCCN2CCC(CN3CC4CC3CN4c3ccc4c(c3)C(=O)N(C3CCC(=O)NC3=O)C4=O)CC2)cc1)c1ccccc1] | 2-(2,6-dioxopiperidin-3-yl)-5-(5-((1-(2-(4-(1,2- diphenylbut-1-en-1-yl)phenoxy)ethyl)piperidin- 4-yl)methyl)-2,5-diazabicyclo[2.2.1]heptan-2- yl)isoindoline-1,3-dione |
| | 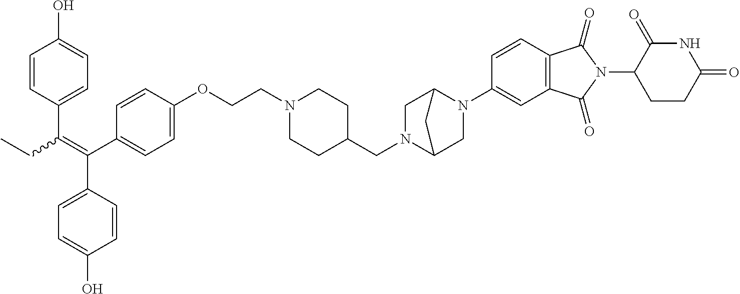 [SMILES: CCC(=C(c1ccc(O)cc1)c1ccc(OCCN2CCC(CN3CC4CC3CN4c3ccc4c(c3)C(=O)N(C3CCC(=O)NC3=O)C4=O)CC2)cc1)c1ccc(O)cc1] | 5-(5-((1-(2-(4-(1,2-bis(4-hydroxyphenyl)but-1- en-1-yl)phenoxy)ethyl)piperidin-4-yl)methyl)- 2,5-diazabicyclo[2.2.1]heptan-2-yl)-2-(2,6- dioxopiperidin-3-yl)isoindoline-1,3-dione |
| | 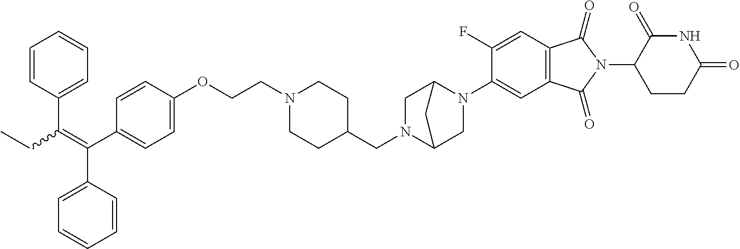 [SMILES: CCC(=C(c1ccccc1)c1ccc(OCCN2CCC(CN3CC4CC3CN4c3cc4c(cc3F)C(=O)N(C3CCC(=O)NC3=O)C4=O)CC2)cc1)c1ccccc1] | 2-(2,6-dioxopiperidin-3-yl)-5-(5-((1-(2-(4-(1,2- diphenylbut-1-en-1-yl)phenoxy)ethyl)piperidin- 4-yl)methyl)-2,5-diazabicyclo[2.2.1]heptan-2- yl)-6-fluoroisoindoline-1,3-dione |
| | 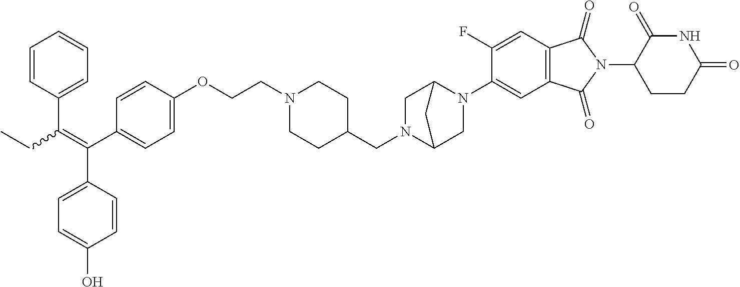 [SMILES: CCC(=C(c1ccc(O)cc1)c1ccc(OCCN2CCC(CN3CC4CC3CN4c3cc4c(cc3F)C(=O)N(C3CCC(=O)NC3=O)C4=O)CC2)cc1)c1ccccc1] | 2-(2,6-dioxopiperidin-3-yl)-5-fluoro-6-(5-((1- (2-(4-(1-(4-hydroxyphenyl)-2-phenylbut-1-en- 1-yl)phenoxy)ethyl)piperidin-4-yl)methyl)-2,5- diazabicyclo[2.2.1]heptan-2-yl)isoindoline-1,3- dione |
| | 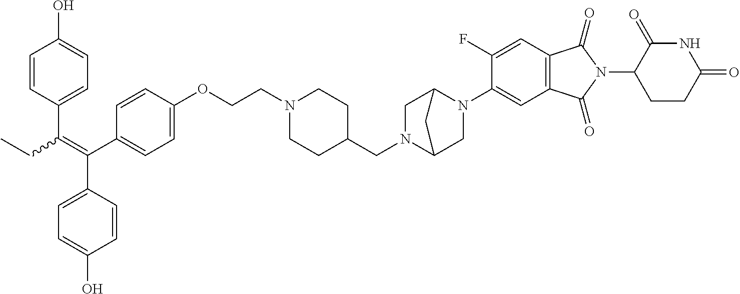 [SMILES: CCC(=C(c1ccc(O)cc1)c1ccc(OCCN2CCC(CN3CC4CC3CN4c3cc4c(cc3F)C(=O)N(C3CCC(=O)NC3=O)C4=O)CC2)cc1)c1ccc(O)cc1] | 5-(5-((1-(2-(4-(1,2-bis(4-hydroxyphenyl)but-1- en-1-yl)phenoxy)ethyl)piperidin-4-yl)methyl)- 2,5-diazabicyclo[2.2.1]heptan-2-yl)-2-(2,6- dioxopiperidin-3-yl)-6-fluoroisoindoline-1,3- dione |
| | 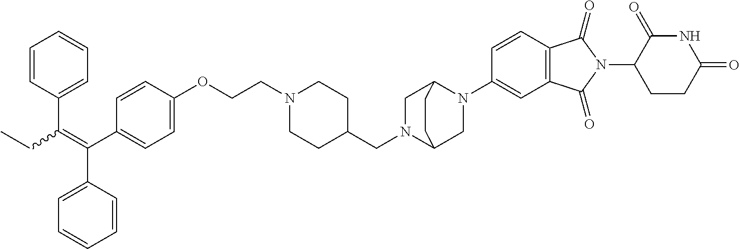 [SMILES: CCC(=C(c1ccccc1)c1ccc(OCCN2CCC(CN3CC4CCC3CN4c3ccc4c(c3)C(=O)N(C3CCC(=O)NC3=O)C4=O)CC2)cc1)c1ccccc1] | 2-(2,6-dioxopiperidin-3-yl)-5-(5-((1-(2-(4-(1,2- diphenylbut-1-en-1-yl)phenoxy)ethyl)piperidin- 4-yl)methyl)-2,5-diazabicyclo[2.2.2]octan-2- yl)isoindoline-1,3-dione |
| | 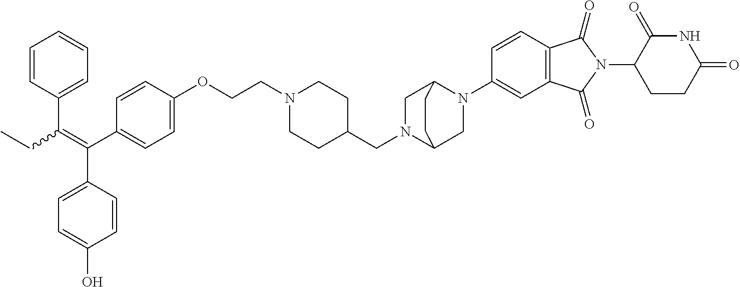 [SMILES: CCC(=C(c1ccc(O)cc1)c1ccc(OCCN2CCC(CN3CC4CCC3CN4c3ccc4c(c3)C(=O)N(C3CCC(=O)NC3=O)C4=O)CC2)cc1)c1ccccc1] | 2-(2,6-dioxopiperidin-3-yl)-5-(5-((1-(2-(4-(1- (4-hydroxyphenyl)-2-phenylbut-1-en-1- yl)phenoxy)ethyl)piperidin-4-yl)methyl)-2,5- diazabicyclo[2.2.2]octan-2-yl)isoindoline-1,3- dione |
| | 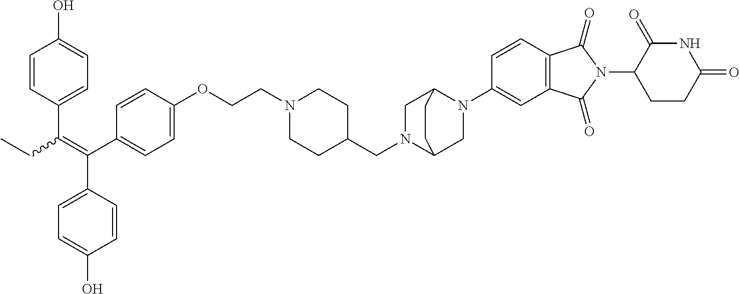 [SMILES: CCC(=C(c1ccc(O)cc1)c1ccc(OCCN2CCC(CN3CC4CCC3CN4c3ccc4c(c3)C(=O)N(C3CCC(=O)NC3=O)C4=O)CC2)cc1)c1ccc(O)cc1] | 5-(5-((1-(2-(4-(1,2-bis(4-hydroxyphenyl)but-1- en-1-yl)phenoxy)ethyl)piperidin-4-yl)methyl)- 2,5-diazabicyclo[2.2.2]octan-2-yl)-2-(2,6- dioxopiperidin-3-yl)isoindoline-1,3-dione |
| | 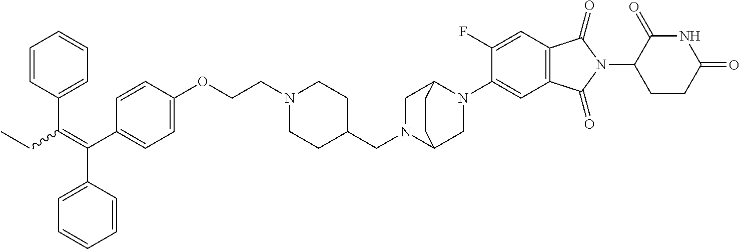 [SMILES: CCC(=C(c1ccccc1)c1ccc(OCCN2CCC(CN3CC4CCC3CN4c3cc4c(cc3F)C(=O)N(C3CCC(=O)NC3=O)C4=O)CC2)cc1)c1ccccc1] | 2-(2,6-dioxopiperidin-3-yl)-5-(5-((1-(2-(4-(1,2- diphenylbut-1-en-1-yl)phenoxy)ethyl)piperidin- 4-yl)methyl)-2,5-diazabicyclo[2.2.2]octan-2- yl)-6-fluoroisoindoline-1,3-dione |
| | 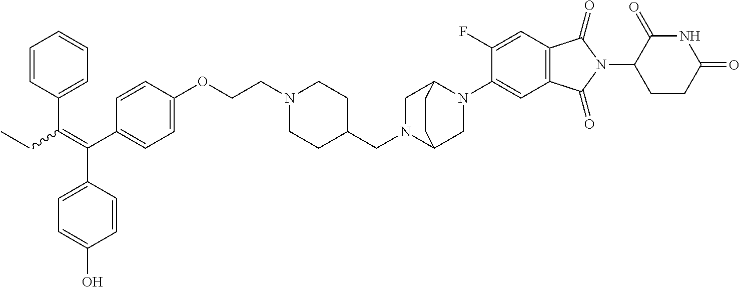 [SMILES: CCC(=C(c1ccc(O)cc1)c1ccc(OCCN2CCC(CN3CC4CCC3CN4c3cc4c(cc3F)C(=O)N(C3CCC(=O)NC3=O)C4=O)CC2)cc1)c1ccccc1] | 2-(2,6-dioxopiperidin-3-yl)-5-fluoro-6-(5-((1- (2-(4-(1-(4-hydroxyphenyl)-2-phenylbut-1-en- 1-yl)phenoxy)ethyl)piperidin-4-yl)methyl)-2,5- diazabicyclo[2.2.2]octan-2-yl)isoindoline-1,3- dione |
| | 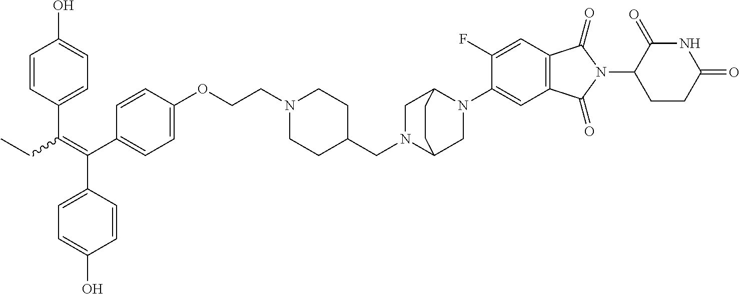 [SMILES: CCC(=C(c1ccc(O)cc1)c1ccc(OCCN2CCC(CN3CC4CCC3CN4c3cc4c(cc3F)C(=O)N(C3CCC(=O)NC3=O)C4=O)CC2)cc1)c1ccc(O)cc1] | 5-(5-((1-(2-(4-(1,2-bis(4-hydroxyphenyl)but-1- en-1-yl)phenoxy)ethyl)piperidin-4-yl)methyl)- 2,5-diazabicyclo[2.2.2]octan-2-yl)-2-(2,6- dioxopiperidin-3-yl)-6-fluoroisoindoline-1,3- dione |
| | 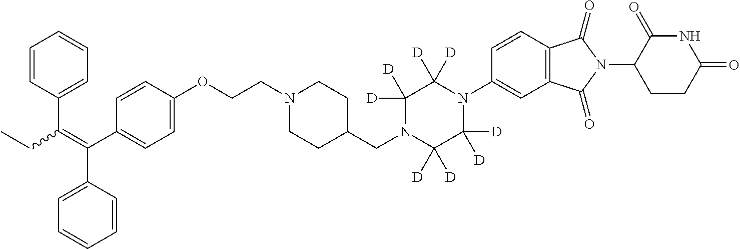 [SMILES: [2H]C1([2H])N(CC2CCN(CCOc3ccc(C(=C(CC)c4ccccc4)c4ccccc4)cc3)CC2)C([2H])([2H])C([2H])([2H])N(c2ccc3c(c2)C(=O)N(C2CCC(=O)NC2=O)C3=O)C1([2H])[2H]] | 2-(2,6-dioxopiperidin-3-yl)-5-(4-((1-(2-(4-(1,2- diphenylbut-1-en-1-yl)phenoxy)ethyl)piperidin- 4-yl)methyl)piperazin-1-yl-2,2,3,3,5,5,6,6- d₈)isoindoline-1,3-dione |
| | 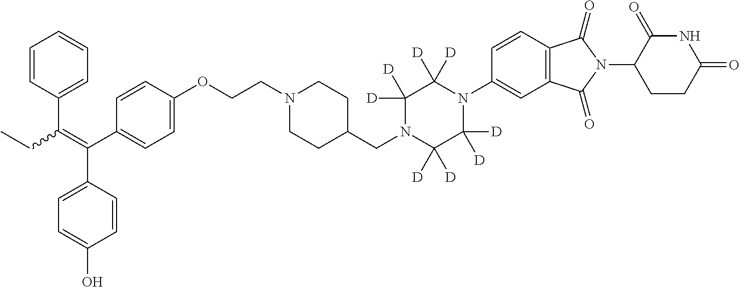 [SMILES: [2H]C1([2H])N(CC2CCN(CCOc3ccc(C(=C(CC)c4ccccc4)c4ccc(O)cc4)cc3)CC2)C([2H])([2H])C([2H])([2H])N(c2ccc3c(c2)C(=O)N(C2CCC(=O)NC2=O)C3=O)C1([2H])[2H]] | 2-(2,6-dioxopiperidin-3-yl)-5-(4-((1-(2-(4-(1- (4-hydroxyphenyl)-2-phenylbut-1-en-1- yl)phenoxy)ethyl)piperidin-4- yl)methyl)piperazin-1-yl-2,2,3,3,5,5,6,6- d₈)isoindoline-1,3-dione |
| | 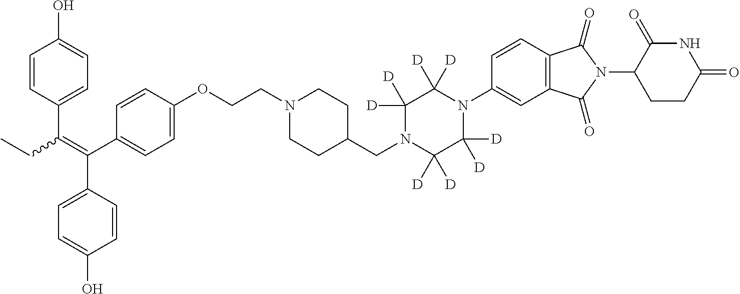 [SMILES: [2H]C1([2H])N(CC2CCN(CCOc3ccc(C(=C(CC)c4ccc(O)cc4)c4ccc(O)cc4)cc3)CC2)C([2H])([2H])C([2H])([2H])N(c2ccc3c(c2)C(=O)N(C2CCC(=O)NC2=O)C3=O)C1([2H])[2H]] | 5-(4-((1-(2-(4-(1,2-bis(4-hydroxyphenyl)but-1- en-1-yl)phenoxy)ethyl)piperidin-4- yl)methyl)piperazin-1-yl-2,2,3,3,5,5,6,6-d₈)-2- (2,6-dioxopiperidin-3-yl)isoindoline-1,3-dione |
| | 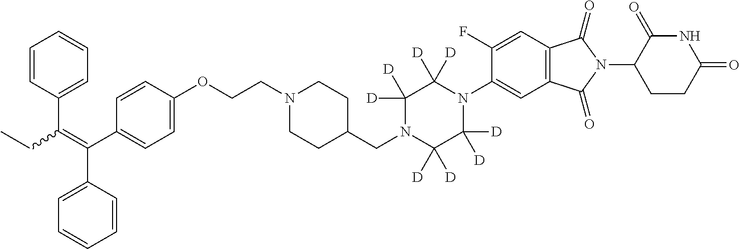 [SMILES: [2H]C1([2H])N(CC2CCN(CCOc3ccc(C(=C(CC)c4ccccc4)c4ccccc4)cc3)CC2)C([2H])([2H])C([2H])([2H])N(c2cc3c(cc2F)C(=O)N(C2CCC(=O)NC2=O)C3=O)C1([2H])[2H]] | 2-(2,6-dioxopiperidin-3-yl)-5-(4-((1-(2-(4-(1,2- diphenylbut-1-en-1-yl)phenoxy)ethyl)piperidin- 4-yl)methyl)piperazin-1-yl-2,2,3,3,5,5,6,6-d₈)- 6-fluoroisoindoline-1,3-dione |
| | 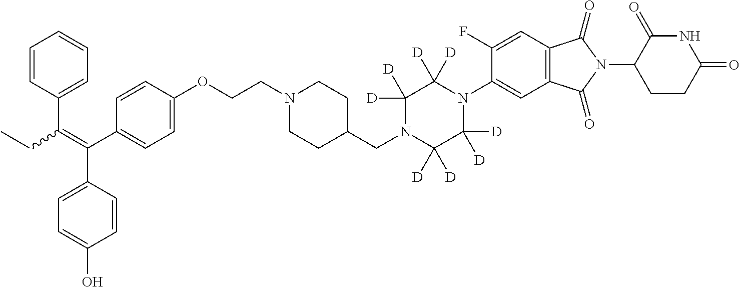 [SMILES: [2H]C1([2H])N(CC2CCN(CCOc3ccc(C(=C(CC)c4ccccc4)c4ccc(O)cc4)cc3)CC2)C([2H])([2H])C([2H])([2H])N(c2cc3c(cc2F)C(=O)N(C2CCC(=O)NC2=O)C3=O)C1([2H])[2H]] | 2-(2,6-dioxopiperidin-3-yl)-5-fluoro-6-(4-((1- (2-(4-(1-(4-hydroxyphenyl)-2-phenylbut-1-en- 1-yl)phenoxy)ethyl)piperidin-4- yl)methyl)piperazin-1-yl-2,2,3,3,5,5,6,6- d₈)isoindoline-1,3-dione |
| | 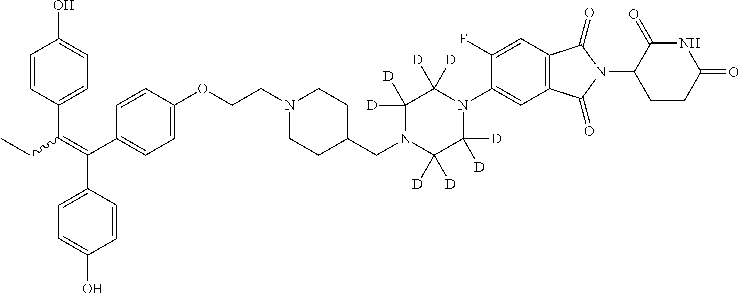 [SMILES: [2H]C1([2H])N(CC2CCN(CCOc3ccc(C(=C(CC)c4ccc(O)cc4)c4ccc(O)cc4)cc3)CC2)C([2H])([2H])C([2H])([2H])N(c2cc3c(cc2F)C(=O)N(C2CCC(=O)NC2=O)C3=O)C1([2H])[2H]] | 5-(4-((1-(2-(4-(1,2-bis(4-hydroxyphenyl)but-1- en-1-yl)phenoxy)ethyl)piperidin-4- yl)methyl)piperazin-1-yl-2,2,3,3,5,5,6,6-d₈)-2- (2,6-dioxopiperidin-3-yl)-6-fluoroisoindoline- 1,3-dione |
| | 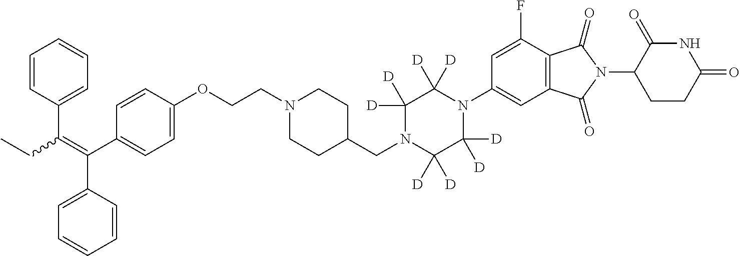 [SMILES: [2H]C1([2H])N(CC2CCN(CCOc3ccc(C(=C(CC)c4ccccc4)c4ccccc4)cc3)CC2)C([2H])([2H])C([2H])([2H])N(c2cc(F)c3c(c2)C(=O)N(C2CCC(=O)NC2=O)C3=O)C1([2H])[2H]] | 2-(2,6-dioxopiperidin-3-yl)-6-(4-((1-(2-(4-(1,2- diphenylbut-1-en-1-yl)phenoxy)ethyl)piperidin- 4-yl)methyl)piperazin-1-yl-2,2,3,3,5,5,6,6-d₈)- 4-fluoroisoindoline-1,3-dione |
| | 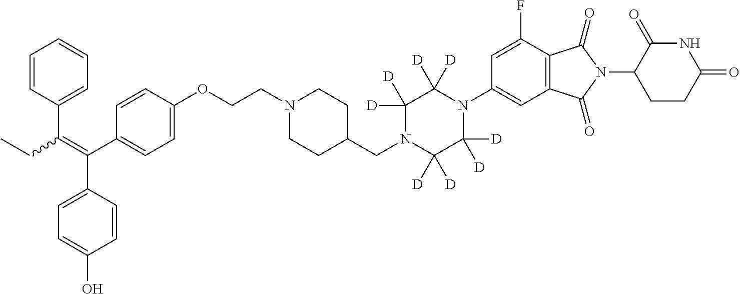 [SMILES: [2H]C1([2H])N(CC2CCN(CCOc3ccc(C(=C(CC)c4ccccc4)c4ccc(O)cc4)cc3)CC2)C([2H])([2H])C([2H])([2H])N(c2cc(F)c3c(c2)C(=O)N(C2CCC(=O)NC2=O)C3=O)C1([2H])[2H]] | 2-(2,6-dioxopiperidin-3-yl)-4-fluoro-6-(4-((1- (2-(4-(1-(4-hydroxyphenyl)-2-phenylbut-1-en- 1-yl)phenoxy)ethyl)piperidin-4- yl)methyl)piperazin-1-yl-2,2,3,3,5,5,6,6- d)isoindoline-1,3-dione |
| | 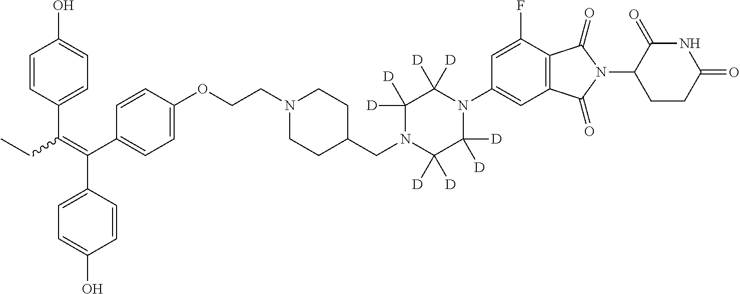 [SMILES: [2H]C1([2H])N(CC2CCN(CCOc3ccc(C(=C(CC)c4ccc(O)cc4)c4ccc(O)cc4)cc3)CC2)C([2H])([2H])C([2H])([2H])N(c2cc(F)c3c(c2)C(=O)N(C2CCC(=O)NC2=O)C3=O)C1([2H])[2H]] | 6-(4-((1-(2-(4-(1,2-bis(4-hydroxyphenyl)but-1- en-1-yl)phenoxy)ethyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 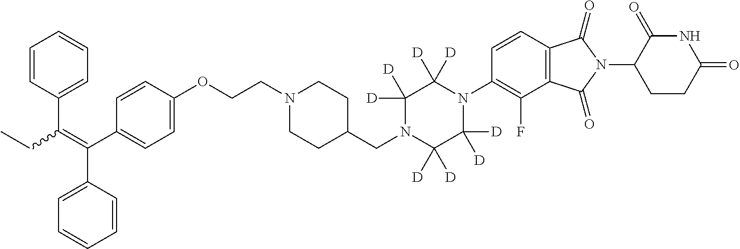 [SMILES: [2H]C1([2H])N(CC2CCN(CCOc3ccc(C(=C(CC)c4ccccc4)c4ccccc4)cc3)CC2)C([2H])([2H])C([2H])([2H])N(c2ccc3c(c2F)C(=O)N(C2CCC(=O)NC2=O)C3=O)C1([2H])[2H]] | 2-(2,6-dioxopiperidin-3-yl)-5-(4-((1-(2-(4-(1,2- diphenylbut-1-en-1-yl)phenoxy)ethyl)piperidin- 4-yl)methyl)piperazin-1-yl-2,2,3,3,5,5,6,6-d₈)- 4-fluoroisoindoline-1,3-dione |
| | 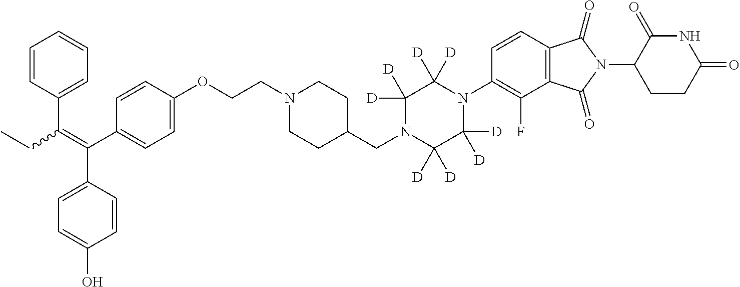 [SMILES: [2H]C1([2H])N(CC2CCN(CCOc3ccc(C(=C(CC)c4ccccc4)c4ccc(O)cc4)cc3)CC2)C([2H])([2H])C([2H])([2H])N(c2ccc3c(c2F)C(=O)N(C2CCC(=O)NC2=O)C3=O)C1([2H])[2H]] | 2-(2,6-dioxopiperidin-3-yl)-4-fluoro-5-(4-((1- (2-(4-(1-(4-hydroxyphenyl)-2-phenylbut-1-en- 1-yl)phenoxy)ethyl)piperidin-4- yl)methyl)piperazin-1-yl-2,2,3,3,5,5,6,6- d₈)isoindoline-1,3-dione |
| | 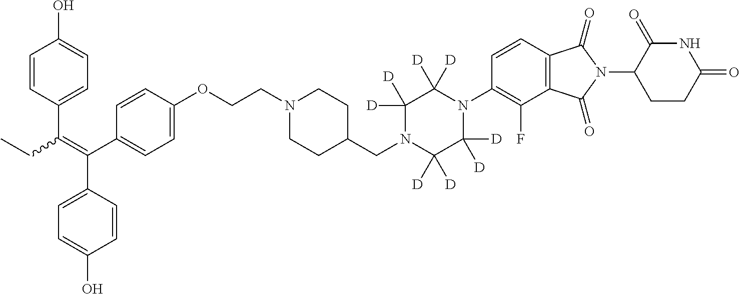 [SMILES: [2H]C1([2H])N(CC2CCN(CCOc3ccc(C(=C(CC)c4ccc(O)cc4)c4ccc(O)cc4)cc3)CC2)C([2H])([2H])C([2H])([2H])N(c2ccc3c(c2F)C(=O)N(C2CCC(=O)NC2=O)C3=O)C1([2H])[2H]] | 5-(4-((1-(2-(4-(1,2-bis(4-hydroxyphenyl)but-1- en-1-yl)phenoxy)ethyl)piperidin-4- yl)methyl)piperazin-1-yl-2,2,3,3,5,5,6,6-d₈)-2- (2,6-dioxopiperidin-3-yl)-4-fluoroisoindoline- 1,3-dione |
| | 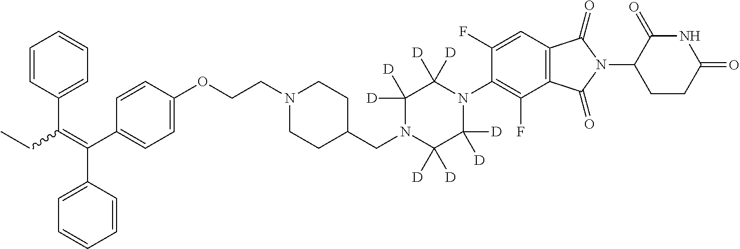 [SMILES: [2H]C1([2H])N(CC2CCN(CCOc3ccc(C(=C(CC)c4ccccc4)c4ccccc4)cc3)CC2)C([2H])([2H])C([2H])([2H])N(c2c(F)cc3c(c2F)C(=O)N(C2CCC(=O)NC2=O)C3=O)C1([2H])[2H]] | 2-(2,6-dioxopiperidin-3-yl)-5-(4-((1-(2-(4-(1,2- diphenylbut-1-en-1-yl)phenoxy)ethyl)piperidin- 4-yl)methyl)piperazin-1-yl-2,2,3,3,5,5,6,6-d₈)- 4,6-difluoroisoindoline-1,3-dione |
| | 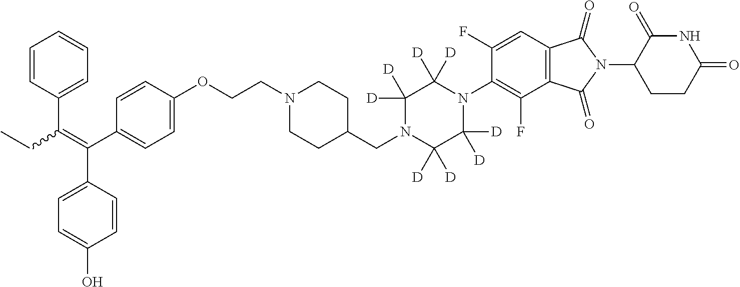 [SMILES: [2H]C1([2H])N(CC2CCN(CCOc3ccc(C(=C(CC)c4ccccc4)c4ccc(O)cc4)cc3)CC2)C([2H])([2H])C([2H])([2H])N(c2c(F)cc3c(c2F)C(=O)N(C2CCC(=O)NC2=O)C3=O)C1([2H])[2H]] | 2-(2,6-dioxopiperidin-3-yl)-4,6-difluoro-5-(4- ((1-(2-(4-(1-(4-hydroxyphenyl)-2-phenylbut-1- en-1-yl)phenoxy)ethyl)piperidin-4- yl)methyl)piperazin-1-yl-2,2,3,3,5,5,6,6- d₈)isoindoline-1,3-dione |
| | 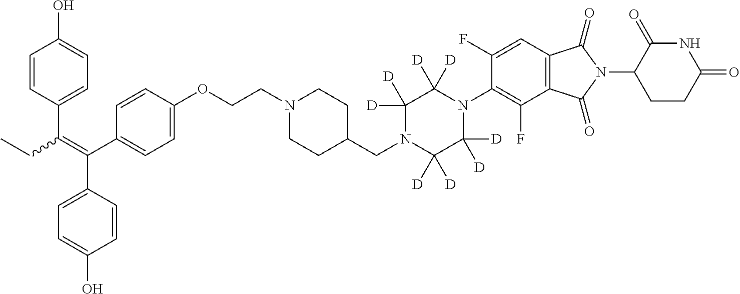 [SMILES: [2H]C1([2H])N(CC2CCN(CCOc3ccc(C(=C(CC)c4ccc(O)cc4)c4ccc(O)cc4)cc3)CC2)C([2H])([2H])C([2H])([2H])N(c2c(F)cc3c(c2F)C(=O)N(C2CCC(=O)NC2=O)C3=O)C1([2H])[2H]] | 5-(4-((1-(2-(4-(1,2-bis(4-hydroxyphenyl)but-1- en-1-yl)phenoxy)ethyl)piperidin-4- yl)methyl)piperazin-1-yl-2,2,3,3,5,5,6,6-d₈)-2- (2,6-dioxopiperidin-3-yl)-4,6- difluoroisoindoline-1,3-dione |
| | 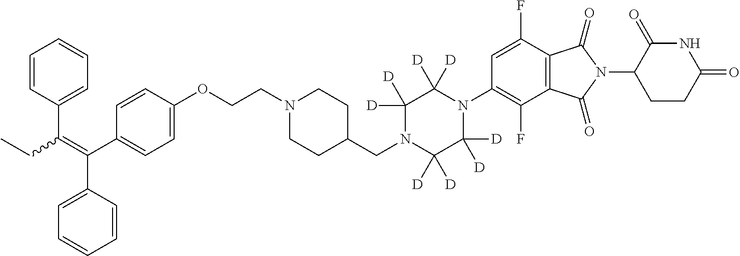 [SMILES: [2H]C1([2H])N(CC2CCN(CCOc3ccc(C(=C(CC)c4ccccc4)c4ccccc4)cc3)CC2)C([2H])([2H])C([2H])([2H])N(c2cc(F)c3c(c2F)C(=O)N(C2CCC(=O)NC2=O)C3=O)C1([2H])[2H]] | 2-(2,6-dioxopiperidin-3-yl)-5-(4-((1-(2-(4-(1,2- diphenylbut-1-en-1-yl)phenoxy)ethyl)piperidin- 4-yl)methyl)piperazin-1-yl-2,2,3,3,5,5,6,6-d₈)- 4,7-difluoroisoindoline-1,3-dione |
| | 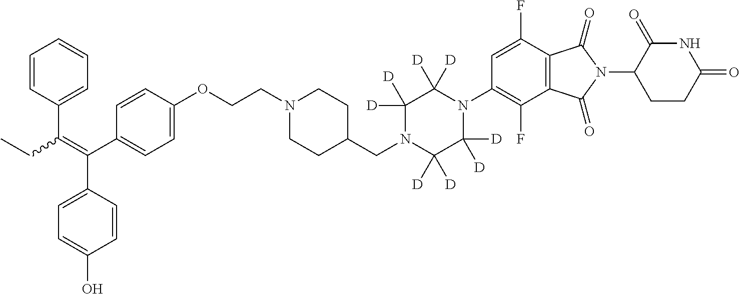 [SMILES: [2H]C1([2H])N(CC2CCN(CCOc3ccc(C(=C(CC)c4ccccc4)c4ccc(O)cc4)cc3)CC2)C([2H])([2H])C([2H])([2H])N(c2cc(F)c3c(c2F)C(=O)N(C2CCC(=O)NC2=O)C3=O)C1([2H])[2H]] | 2-(2,6-dioxopiperidin-3-yl)-4,7-difluoro-5-(4- ((1-(2-(4-(1-(4-hydroxyphenyl)-2-phenylbut-1- en-1-yl)phenoxy)ethyl)piperidin-4- yl)methyl)piperazin-1-yl-2,2,3,3,5,5,6,6- d₈)isoindoline-1,3-dione |
| | 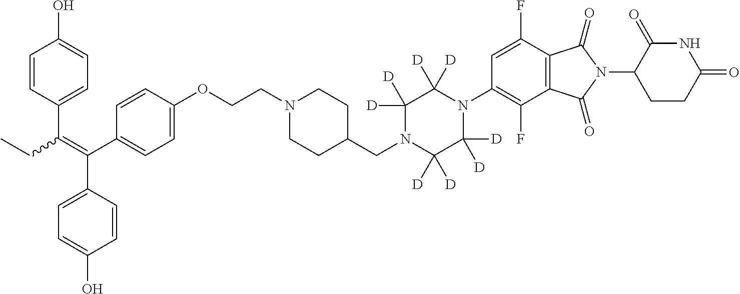 [SMILES: [2H]C1([2H])N(CC2CCN(CCOc3ccc(C(=C(CC)c4ccc(O)cc4)c4ccc(O)cc4)cc3)CC2)C([2H])([2H])C([2H])([2H])N(c2cc(F)c3c(c2F)C(=O)N(C2CCC(=O)NC2=O)C3=O)C1([2H])[2H]] | 5-(4-((1-(2-(4-(1,2-bis(4-hydroxyphenyl)but-1- en-1-yl)phenoxy)ethyl)piperidin-4- yl)methyl)piperazin-1-yl-2,2,3,3,5,5,6,6-d₈)-2- (2,6-dioxopiperidin-3-yl)-4,7- difluoroisoindoline-1,3-dione |
| | 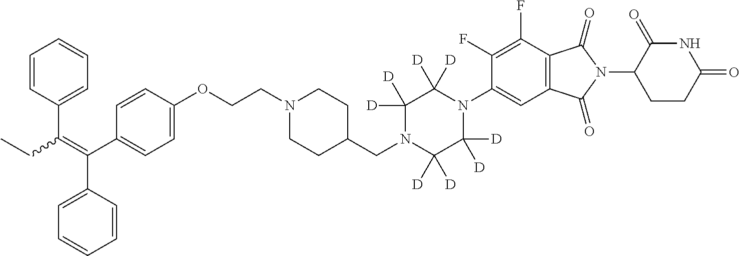 [SMILES: [2H]C1([2H])N(CC2CCN(CCOc3ccc(C(=C(CC)c4ccccc4)c4ccccc4)cc3)CC2)C([2H])([2H])C([2H])([2H])N(c2cc3c(c(F)c2F)C(=O)N(C2CCC(=O)NC2=O)C3=O)C1([2H])[2H]] | 2-(2,6-dioxopiperidin-3-yl)-6-(4-((1-(2-(4-(1,2- diphenylbut-1-en-1-yl)phenoxy)ethyl)piperidin- 4-yl)methyl)piperazin-1-yl-2,2,3,3,5,5,6,6-d₈)- 4,5-difluoroisoindoline-1,3-dione |
| | 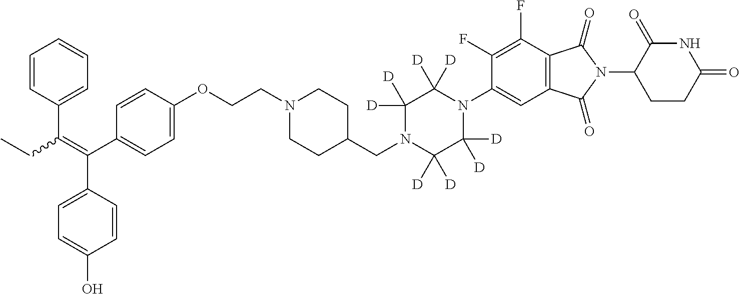 [SMILES: [2H]C1([2H])N(CC2CCN(CCOc3ccc(C(=C(CC)c4ccccc4)c4ccc(O)cc4)cc3)CC2)C([2H])([2H])C([2H])([2H])N(c2cc3c(c(F)c2F)C(=O)N(C2CCC(=O)NC2=O)C3=O)C1([2H])[2H]] | 2-(2,6-dioxopiperidin-3-yl)-4,5-difluoro-6-(4- ((1-(2-(4-(1-(4-hydroxyphenyl)-2-phenylbut-1- en-1-yl)phenoxy)ethyl)piperidin-4- yl)methyl)piperazin-1-yl-2,2,3,3,5,5,6,6- d₈)isoindoline-1,3-dione |
| | 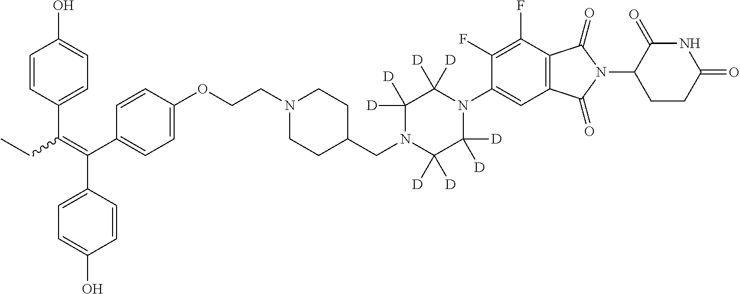 [SMILES: [2H]C1([2H])N(CC2CCN(CCOc3ccc(C(=C(CC)c4ccc(O)cc4)c4ccc(O)cc4)cc3)CC2)C([2H])([2H])C([2H])([2H])N(c2cc3c(c(F)c2F)C(=O)N(C2CCC(=O)NC2=O)C3=O)C1([2H])[2H]] | 6-(4-((1-(2-(4-(1,2-bis(4-hydroxyphenyl)but-1- en-1-yl)phenoxy)ethyl)piperidin-4- yl)methyl)piperazin-1-yl-2,2,3,3,5,5,6,6-d₈)-2- (2,6-dioxopiperidin-3-yl)-4,5- difluoroisoindoline-1,3-dione |
| | 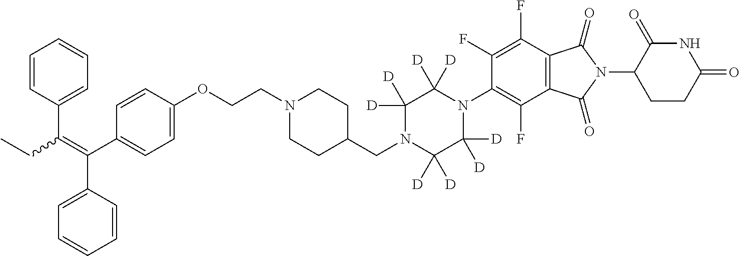 [SMILES: [2H]C1([2H])N(CC2CCN(CCOc3ccc(C(=C(CC)c4ccccc4)c4ccccc4)cc3)CC2)C([2H])([2H])C([2H])([2H])N(c2c(F)c(F)c3c(c2F)C(=O)N(C2CCC(=O)NC2=O)C3=O)C1([2H])[2H]] | 2-(2,6-dioxopiperidin-3-yl)-5-(4-((1-(2-(4-(1,2- diphenylbut-1-en-1-yl)phenoxy)ethyl)piperidin- 4-yl)methyl)piperazin-1-yl-2,2,3,3,5,5,6,6-d₈)- 4,6,7-trifluoroisoindoline-1,3-dione |
| | 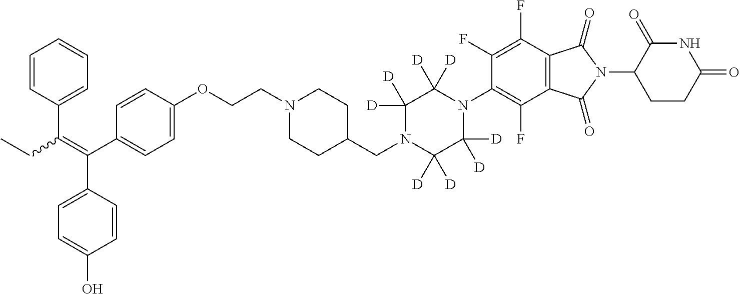 [SMILES: [2H]C1([2H])N(CC2CCN(CCOc3ccc(C(=C(CC)c4ccccc4)c4ccc(O)cc4)cc3)CC2)C([2H])([2H])C([2H])([2H])N(c2c(F)c(F)c3c(c2F)C(=O)N(C2CCC(=O)NC2=O)C3=O)C1([2H])[2H]] | 2-(2,6-dioxopiperidin-3-yl)-4,5,7-trifluoro-6- (4-((1-(2-(4-(1-(4-hydroxyphenyl)-2-phenylbut- 1-en-1-yl)phenoxy)ethyl)piperidin-4- yl)methyl)piperazin-1-yl-2,2,3,3,5,5,6,6- d₈)isoindoline-1,3-dione |
| | 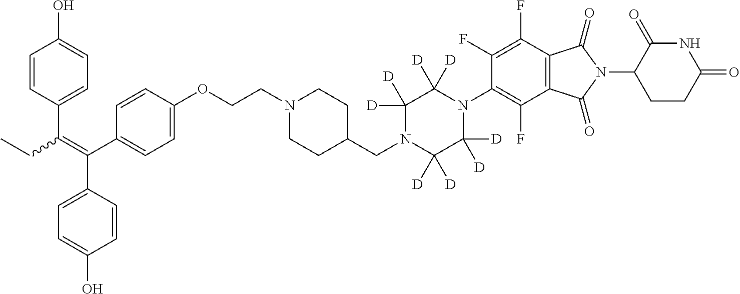 [SMILES: [2H]C1([2H])N(CC2CCN(CCOc3ccc(C(=C(CC)c4ccc(O)cc4)c4ccc(O)cc4)cc3)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(2-(4-(1,2-bis(4-hydroxyphenyl)but-1- en-1-yl)phenoxy)ethyl)piperidin-4- yl)methyl)piperazin-1-yl-2,2,3,3,5,5,6,6-d₈)-2- (2,6-dioxopiperidin-3-yl)-4,6,7- trifluoroisoindoline-1,3-dione |
| | 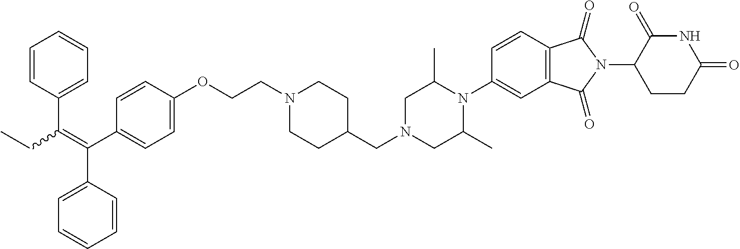 [SMILES: CCC(=C(c1ccccc1)c1ccc(OCCN2CCC(CN3CC(C)N(c4ccc5c(c4)C(=O)N(C4CCC(=O)NC4=O)C5=O)C(C)C3)CC2)cc1)c1ccccc1] | 2-(2,6-dioxopiperidin-3-yl)-5-(4-((1-(2-(4-(1,2- diphenylbut-1-en-1-yl)phenoxy)ethyl)piperidin- 4-yl)methyl)-2,6-dimethylpiperazin-1- yl)isoindoline-1,3-dione |
| | 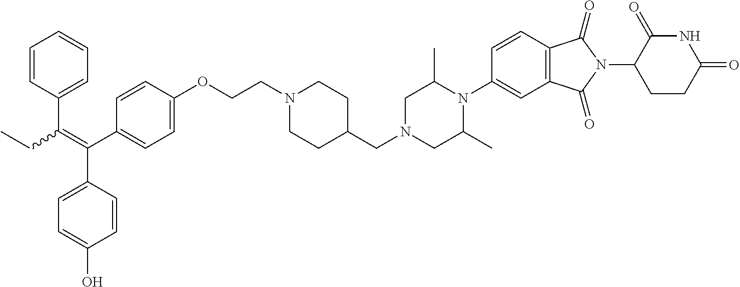 [SMILES: CCC(=C(c1ccc(O)cc1)c1ccc(OCCN2CCC(CN3CC(C)N(c4ccc5c(c4)C(=O)N(C4CCC(=O)NC4=O)C5=O)C(C)C3)CC2)cc1)c1ccccc1] | 2-(2,6-dioxopiperidin-3-yl)-5-(4-((1-(2-(4-(1- (4-hydroxyphenyl)-2-phenylbut-1-en-1- yl)phenoxy)ethyl)piperidin-4-yl)methyl)-2,6- dimethylpiperazin-1-yl)isoindoline-1,3-dione |
| | 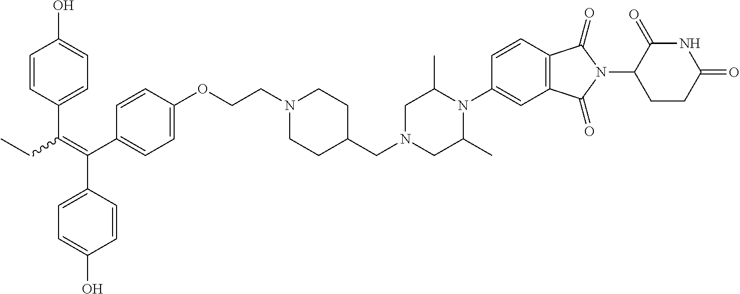 [SMILES: CCC(=C(c1ccc(O)cc1)c1ccc(OCCN2CCC(CN3CC(C)N(c4ccc5c(c4)C(=O)N(C4CCC(=O)NC4=O)C5=O)C(C)C3)CC2)cc1)c1ccc(O)cc1] | 5-(4-((1-(2-(4-(1,2-bis(4-hydroxyphenyl)but-1- en-1-yl)phenoxy)ethyl)piperidin-4-yl)methyl)- 2,6-dimethylpiperazin-1-yl)-2-(2,6- dioxopiperidin-3-yl)isoindoline-1,3-dione |
| | 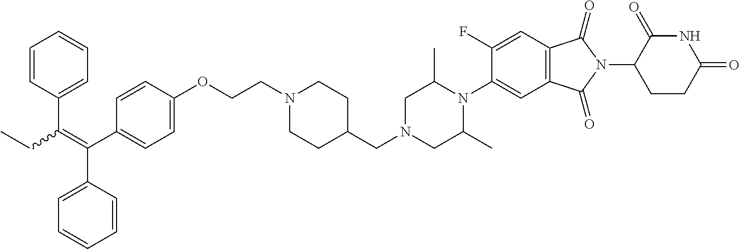 [SMILES: CCC(=C(c1ccccc1)c1ccc(OCCN2CCC(CN3CC(C)N(c4cc5c(cc4F)C(=O)N(C4CCC(=O)NC4=O)C5=O)C(C)C3)CC2)cc1)c1ccccc1] | 2-(2,6-dioxopiperidin-3-yl)-5-(4-((1-(2-(4-(1,2- diphenylbut-1-en-1-yl)phenoxy)ethyl)piperidin- 4-yl)methyl)-2,6-dimethylpiperazin-1-yl)-6- fluoroisoindoline-1,3-dione |
| | 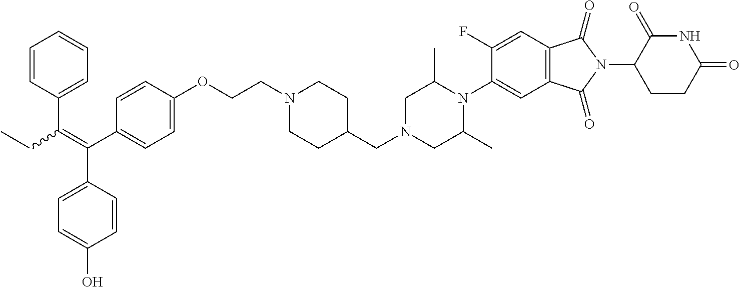 [SMILES: CCC(=C(c1ccc(O)cc1)c1ccc(OCCN2CCC(CN3CC(C)N(c4cc5c(cc4F)C(=O)N(C4CCC(=O)NC4=O)C5=O)C(C)C3)CC2)cc1)c1ccccc1] | 2-(2,6-dioxopiperidin-3-yl)-5-fluoro-6-(4-((1- (2-(4-(1-(4-hydroxyphenyl)-2-phenylbut-1-en- 1-yl)phenoxy)ethyl)piperidin-4-yl)methyl)-2,6- dimethylpiperazin-1-yl)isoindoline-1,3-dione |
| | 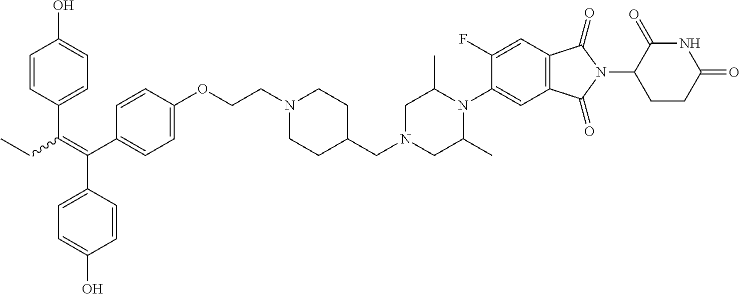 [SMILES: CCC(=C(c1ccc(O)cc1)c1ccc(OCCN2CCC(CN3CC(C)N(c4cc5c(cc4F)C(=O)N(C4CCC(=O)NC4=O)C5=O)C(C)C3)CC2)cc1)c1ccc(O)cc1] | 5-(4-((1-(2-(4-(1,2-bis(4-hydroxyphenyl)but-1- en-1-yl)phenoxy)ethyl)piperidin-4-yl)methyl)- 2,6-dimethylpiperazin-1-yl)-2-(2,6- dioxopiperidin-3-yl)-6-fluoroisoindoline-1,3- dione |
| | 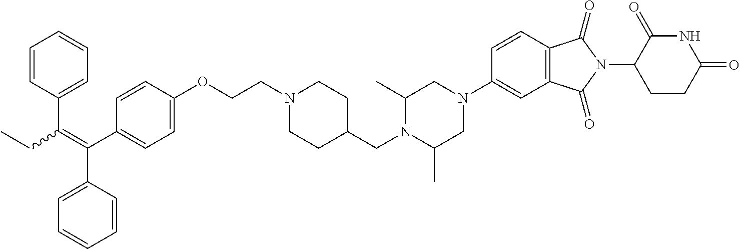 [SMILES: CCC(=C(c1ccccc1)c1ccc(OCCN2CCC(CN3C(C)CN(c4ccc5c(c4)C(=O)N(C4CCC(=O)NC4=O)C5=O)CC3C)CC2)cc1)c1ccccc1] | 2-(2,6-dioxopiperidin-3-yl)-5-(4-((1-(2-(4-(1,2- diphenylbut-1-en-1-yl)phenoxy)ethyl)piperidin- 4-yl)methyl)-3,5-dimethylpiperazin-1- yl)isoindoline-1,3-dione |
| | 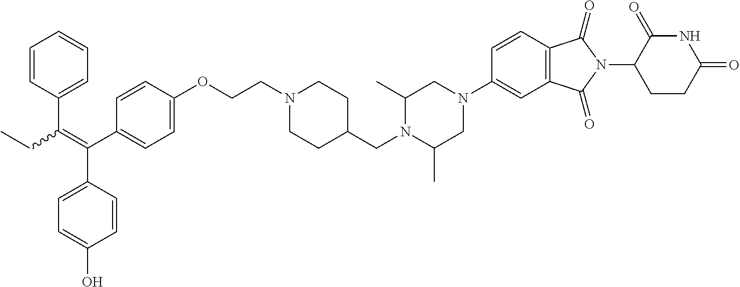 [SMILES: CCC(=C(c1ccc(O)cc1)c1ccc(OCCN2CCC(CN3C(C)CN(c4ccc5c(c4)C(=O)N(C4CCC(=O)NC4=O)C5=O)CC3C)CC2)cc1)c1ccccc1] | 2-(2,6-dioxopiperidin-3-yl)-5-(4-((1-(2-(4-(1- (4-hydroxyphenyl)-2-phenylbut-1-en-1- yl)phenoxy)ethyl)piperidin-4-yl)methyl)-3,5- dimethylpiperazin-1-yl)isoindoline-1,3-dione |
| | 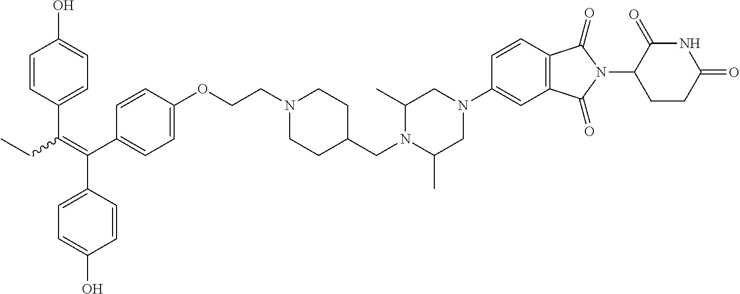 [SMILES: CCC(=C(c1ccc(O)cc1)c1ccc(OCCN2CCC(CN3C(C)CN(c4ccc5c(c4)C(=O)N(C4CCC(=O)NC4=O)C5=O)CC3C)CC2)cc1)c1ccc(O)cc1] | 5-(4-((1-(2-(4-(1,2-bis(4-hydroxyphenyl)but-1- en-1-yl)phenoxy)ethyl)piperidin-4-yl)methyl)- 3,5-dimethylpiperazin-1-yl)-2-(2,6- dioxopiperidin-3-yl)isoindoline-1,3-dione |
| | 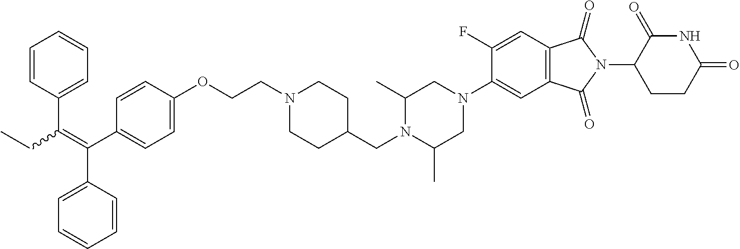 [SMILES: CCC(=C(c1ccccc1)c1ccc(OCCN2CCC(CN3C(C)CN(c4cc5c(cc4F)C(=O)N(C4CCC(=O)NC4=O)C5=O)CC3C)CC2)cc1)c1ccccc1] | 2-(2,6-dioxopiperidin-3-yl)-5-(4-((1-(2-(4-(1,2- diphenylbut-1-en-1-yl)phenoxy)ethyl)piperidin- 4-yl)methyl)-3,5-dimethylpiperazin-1-yl)-6- fluoroisoindoline-1,3-dione |
| | 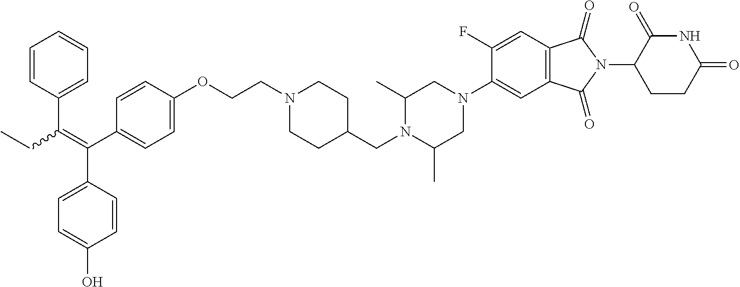 [SMILES: CCC(=C(c1ccc(O)cc1)c1ccc(OCCN2CCC(CN3C(C)CN(c4cc5c(cc4F)C(=O)N(C4CCC(=O)NC4=O)C5=O)CC3C)CC2)cc1)c1ccccc1] | 2-(2,6-dioxopiperidin-3-yl)-5-fluoro-6-(4-((1- (2-(4-(1-(4-hydroxyphenyl)-2-phenylbut-1-en- 1-yl)phenoxy)ethyl)piperidin-4-yl)methyl)-3,5- dimethylpiperazin-1-yl)isoindoline-1,3-dione |
| | 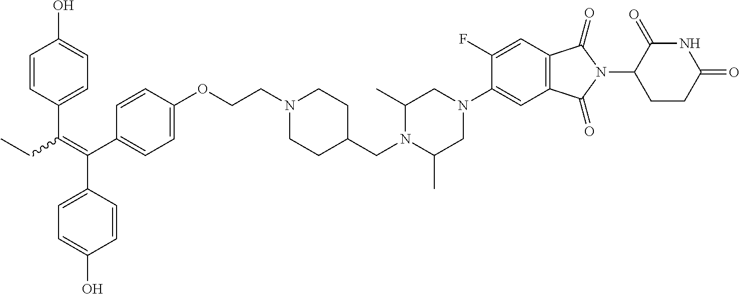 [SMILES: CCC(=C(c1ccc(O)cc1)c1ccc(OCCN2CCC(CN3C(C)CN(c4cc5c(cc4F)C(=O)N(C4CCC(=O)NC4=O)C5=O)CC3C)CC2)cc1)c1ccc(O)cc1] | 5-(4-((1-(2-(4-(1,2-bis(4-hydroxyphenyl)but-1- en-1-yl)phenoxy)ethyl)piperidin-4-yl)methyl)- 3,5-dimethylpiperazin-1-yl)-2-(2,6- dioxopiperidin-3-yl)-6-fluoroisoindoline-1,3- dione |
| | 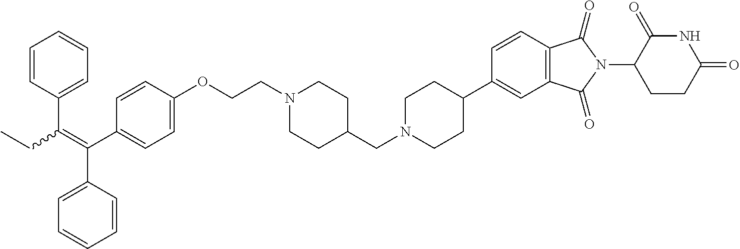 [SMILES: CCC(=C(c1ccccc1)c1ccc(OCCN2CCC(CN3CCC(c4ccc5c(c4)C(=O)N(C4CCC(=O)NC4=O)C5=O)CC3)CC2)cc1)c1ccccc1] | 2-(2,6-dioxopiperidin-3-yl)-5-(1-((1-(2-(4-(1,2- diphenylbut-1-en-1-yl)phenoxy)ethyl)piperidin- 4-yl)methyl)piperidin-4-yl)isoindoline-1,3- dione |
| | 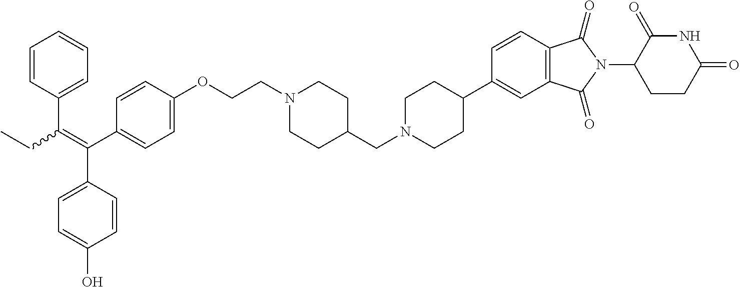 [SMILES: CCC(=C(c1ccc(O)cc1)c1ccc(OCCN2CCC(CN3CCC(c4ccc5c(c4)C(=O)N(C4CCC(=O)NC4=O)C5=O)CC3)CC2)cc1)c1ccccc1] | 2-(2,6-dioxopiperidin-3-yl)-5-(1-((1-(2-(4-(1- (4-hydroxyphenyl)-2-phenylbut-1-en-1- yl)phenoxy)ethyl)piperidin-4- yl)methyl)piperidin-4-yl)isoindoline-1,3-dione |
| | 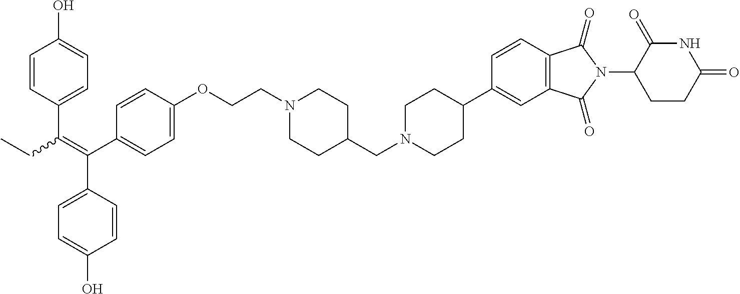 [SMILES: CCC(=C(c1ccc(O)cc1)c1ccc(OCCN2CCC(CN3CCC(c4ccc5c(c4)C(=O)N(C4CCC(=O)NC4=O)C5=O)CC3)CC2)cc1)c1ccc(O)cc1] | 5-(1-((1-(2-(4-(1,2-bis(4-hydroxyphenyl)but-1- en-1-yl)phenoxy)ethyl)piperidin-4- yl)methyl)piperidin-4-yl)-2-(2,6- dioxopiperidin-3-yl)isoindoline-1,3-dione |
| | 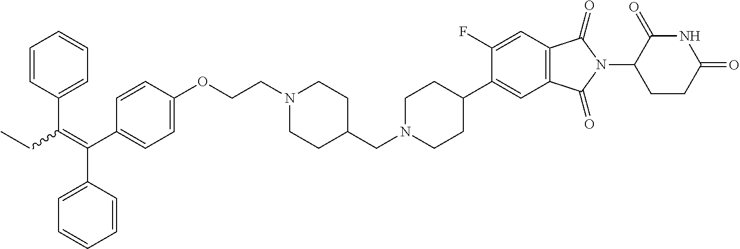 [SMILES: CCC(=C(c1ccccc1)c1ccc(OCCN2CCC(CN3CCC(c4cc5c(cc4F)C(=O)N(C4CCC(=O)NC4=O)C5=O)CC3)CC2)cc1)c1ccccc1] | 2-(2,6-dioxopiperidin-3-yl)-5-(1-((1-(2-(4-(1,2- diphenylbut-1-en-1-yl)phenoxy)ethyl)piperidin- 4-yl)methyl)piperidin-4-yl)-6- fluoroisoindoline-1,3-dione |
| | 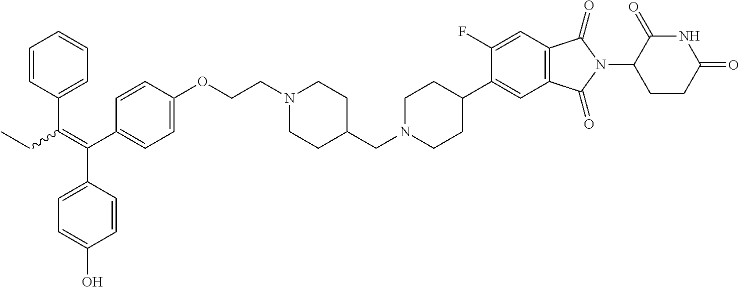 [SMILES: CCC(=C(c1ccc(O)cc1)c1ccc(OCCN2CCC(CN3CCC(c4cc5c(cc4F)C(=O)N(C4CCC(=O)NC4=O)C5=O)CC3)CC2)cc1)c1ccccc1] | 2-(2,6-dioxopiperidin-3-yl)-5-fluoro-6-(1-((1- (2-(4-(1-(4-hydroxyphenyl)-2-phenylbut-1-en- 1-yl)phenoxy)ethyl)piperidin-4- yl)methyl)piperidin-4-yl)isoindoline-1,3-dione |
| | 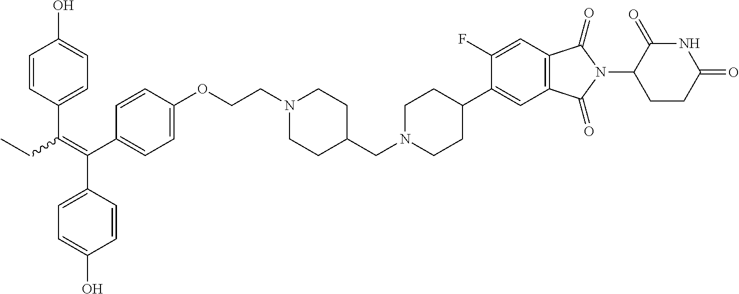 [SMILES: CCC(=C(c1ccc(O)cc1)c1ccc(OCCN2CCC(CN3CCC(c4cc5c(cc4F)C(=O)N(C4CCC(=O)NC4=O)C5=O)CC3)CC2)cc1)c1ccc(O)cc1] | 5-(1-((1-(2-(4-(1,2-bis(4-hydroxyphenyl)but-1- en-1-yl)phenoxy)ethyl)piperidin-4- yl)methyl)piperidin-4-yl)-2-(2,6- dioxopiperidin-3-yl)-6-fluoroisoindoline-1,3- dione |
| | 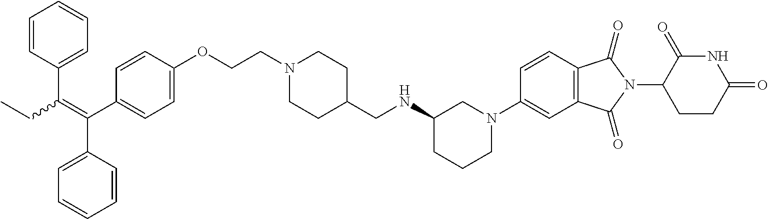 [SMILES: CCC(=C(c1ccccc1)c1ccc(OCCN2CCC(CN[C@@H]3CCCN(c4ccc5c(c4)C(=O)N(C4CCC(=O)NC4=O)C5=O)C3)CC2)cc1)c1ccccc1] | 2-(2,6-dioxopiperidin-3-yl)-5-((R)-3-(((1-(2-(4- (1,2-diphenylbut-1-en-1- yl)phenoxy)ethyl)piperidin-4- yl)methyl)amino)piperidin-1-yl)isoindoline- 1,3-dione |
| | 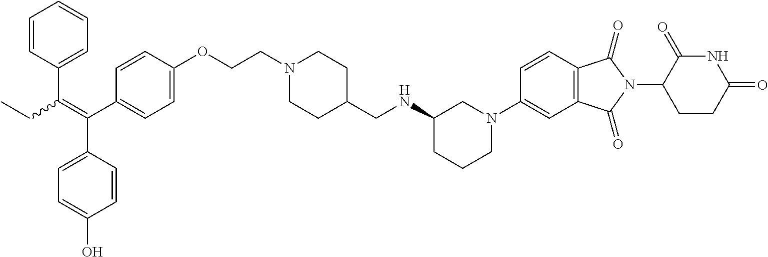 [SMILES: CCC(=C(c1ccc(O)cc1)c1ccc(OCCN2CCC(CN[C@@H]3CCCN(c4ccc5c(c4)C(=O)N(C4CCC(=O)NC4=O)C5=O)C3)CC2)cc1)c1ccccc1] | 2-(2,6-dioxopiperidin-3-yl)-5-((R)-3-(((1-(2-(4- (1-(4-hydroxyphenyl)-2-phenylbut-1-en-1- yl)phenoxy)ethyl)piperidin-4- yl)methyl)amino)piperidin-1-yl)isoindoline- 1,3-dione |
| | 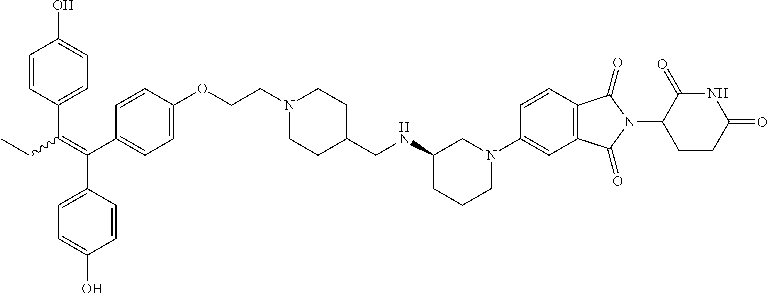 [SMILES: CCC(=C(c1ccc(O)cc1)c1ccc(OCCN2CCC(CN[C@@H]3CCCN(c4ccc5c(c4)C(=O)N(C4CCC(=O)NC4=O)C5=O)C3)CC2)cc1)c1ccc(O)cc1] | 5-((R)-3-(((1-(2-(4-(1,2-bis(4- hydroxyphenyl)but-1-en-1- yl)phenoxy)ethyl)piperidin-4- yl)methyl)amino)piperidin-1-yl)-2-(2,6- dioxopiperidin-3-yl)isoindoline-1,3-dione |
| | 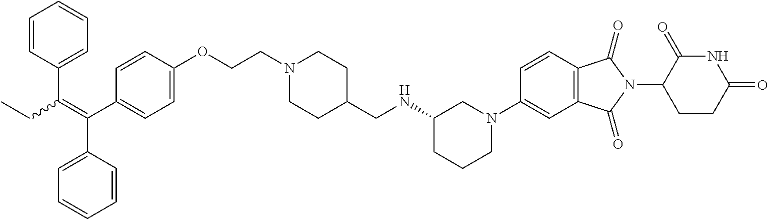 [SMILES: CCC(=C(c1ccccc1)c1ccc(OCCN2CCC(CN[C@H]3CCCN(c4ccc5c(c4)C(=O)N(C4CCC(=O)NC4=O)C5=O)C3)CC2)cc1)c1ccccc1] | 2-(2,6-dioxopiperidin-3-yl)-5-((S)-3-(((1-(2-(4- (1,2-diphenylbut-1-en-1- yl)phenoxy)ethyl)piperidin-4- yl)methyl)amino)piperidin-1-yl)isoindoline- 1,3-dione |
| | 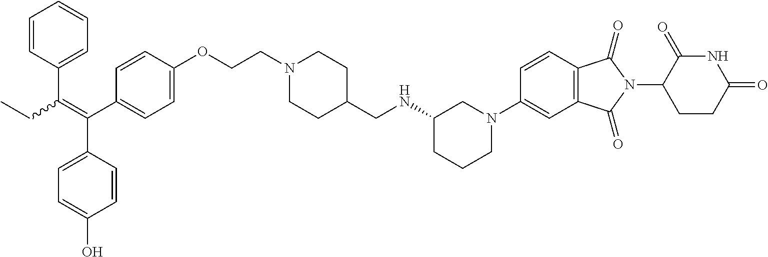 [SMILES: CCC(=C(c1ccc(O)cc1)c1ccc(OCCN2CCC(CN[C@H]3CCCN(c4ccc5c(c4)C(=O)N(C4CCC(=O)NC4=O)C5=O)C3)CC2)cc1)c1ccccc1] | 2-(2,6-dioxopiperidin-3-yl)-5-((S)-3-(((1-(2-(4- (1-(4-hydroxyphenyl)-2-phenylbut-1-en-1- yl)phenoxy)ethyl)piperidin-4- yl)methyl)amino)piperidin-1-yl)isoindoline- 1,3-dione |
| | 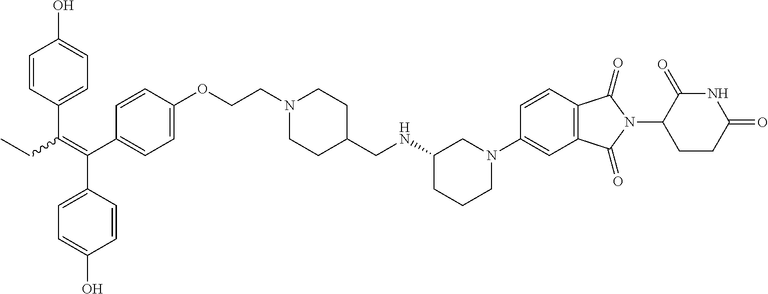 [SMILES: CCC(=C(c1ccc(O)cc1)c1ccc(OCCN2CCC(CN[C@H]3CCCN(c4ccc5c(c4)C(=O)N(C4CCC(=O)NC4=O)C5=O)C3)CC2)cc1)c1ccc(O)cc1] | 5-((S)-3-(((1-(2-(4-(1,2-bis(4- hydroxyphenyl)but-1-en-1- yl)phenoxy)ethyl)piperidin-4- yl)methyl)amino)piperidin-1-yl)-2-(2,6- dioxopiperidin-3-yl)isoindoline-1,3-dione |
| | 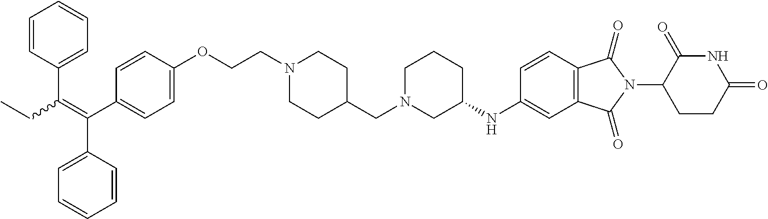 [SMILES: CCC(=C(c1ccccc1)c1ccc(OCCN2CCC(CN3CCC[C@H](Nc4ccc5c(c4)C(=O)N(C4CCC(=O)NC4=O)C5=O)C3)CC2)cc1)c1ccccc1] | 2-(2,6-dioxopiperidin-3-yl)-5-(((S)-1-((1-(2-(4- (1,2-diphenylbut-1-en-1- yl)phenoxy)ethyl)piperidin-4- yl)methyl)piperidin-3-yl)amino)isoindoline- 1,3-dione |
| | 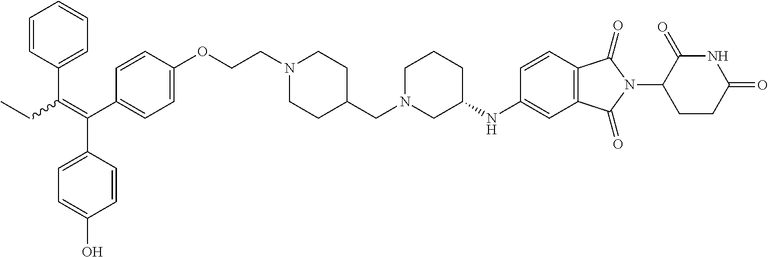 [SMILES: CCC(=C(c1ccc(O)cc1)c1ccc(OCCN2CCC(CN3CCC[C@H](Nc4ccc5c(c4)C(=O)N(C4CCC(=O)NC4=O)C5=O)C3)CC2)cc1)c1ccccc1] | 2-(2,6-dioxopiperidin-3-yl)-5-(((S)-1-((1-(2-(4- (1-(4-hydroxyphenyl)-2-phenylbut-1-en-1- yl)phenoxy)ethyl)piperidin-4- yl)methyl)piperidin-3-yl)amino)isoindoline- 1,3-dione |
| | 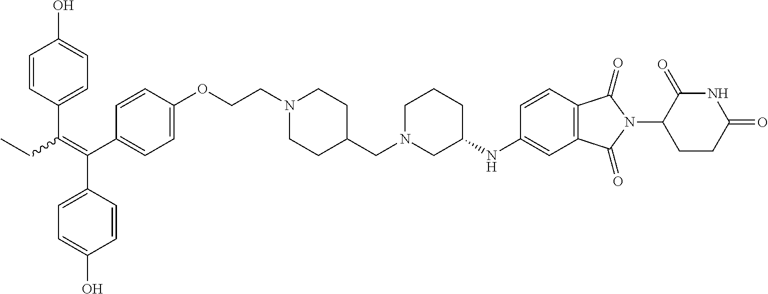 [SMILES: CCC(=C(c1ccc(O)cc1)c1ccc(OCCN2CCC(CN3CCC[C@H](Nc4ccc5c(c4)C(=O)N(C4CCC(=O)NC4=O)C5=O)C3)CC2)cc1)c1ccc(O)cc1] | 5-(((S)-1-((1-(2-(4-(1,2-bis(4- hydroxyphenyl)but-1-en-1- yl)phenoxy)ethyl)piperidin-4- yl)methyl)piperidin-3-yl)amino)-2-(2,6- dioxopiperidin-3-yl)isoindoline-1,3-dione |
| | 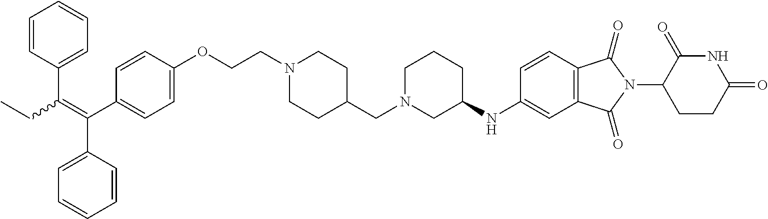 [SMILES: CCC(=C(c1ccccc1)c1ccc(OCCN2CCC(CN3CCC[C@@H](Nc4ccc5c(c4)C(=O)N(C4CCC(=O)NC4=O)C5=O)C3)CC2)cc1)c1ccccc1] | 2-(2,6-dioxopiperidin-3-yl)-5-(((R)-1-((1-(2-(4- (1,2-diphenylbut-1-en-1- yl)phenoxy)ethyl)piperidin-4- yl)methyl)piperidin-3-yl)amino)isoindoline- 1,3-dione |
| | 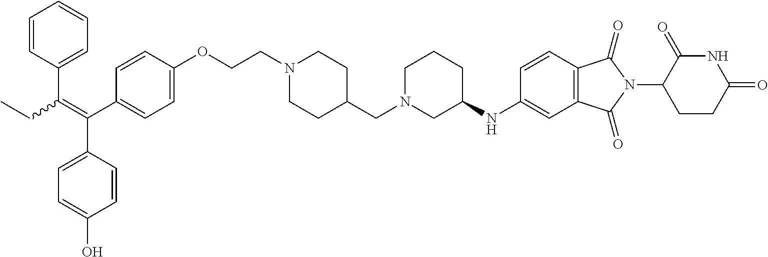 [SMILES: CCC(=C(c1ccc(O)cc1)c1ccc(OCCN2CCC(CN3CCC[C@@H](Nc4ccc5c(c4)C(=O)N(C4CCC(=O)NC4=O)C5=O)C3)CC2)cc1)c1ccccc1] | 2-(2,6-dioxopiperidin-3-yl)-5-(((R)-1-((1-(2-(4- (1-(4-hydroxyphenyl)-2-phenylbut-1-en-1- yl)phenoxy)ethyl)piperidin-4- yl)methyl)piperidin-3-yl)amino)isoindoline- 1,3-dione |
| | 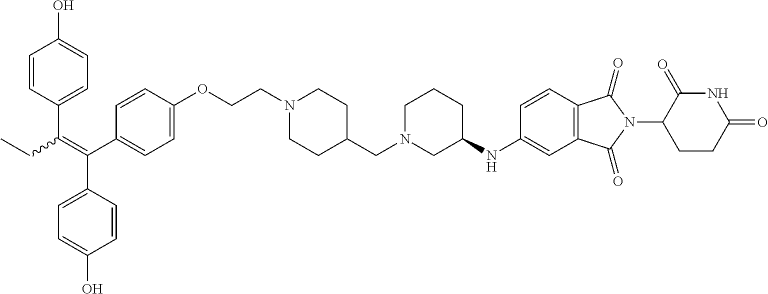 [SMILES: CCC(=C(c1ccc(O)cc1)c1ccc(OCCN2CCC(CN3CCC[C@@H](Nc4ccc5c(c4)C(=O)N(C4CCC(=O)NC4=O)C5=O)C3)CC2)cc1)c1ccc(O)cc1] | 5-(((R)-1-((1-(2-(4-(1,2-bis(4- hydroxyphenyl)but-1-en-1- yl)phenoxy)ethyl)piperidin-4- yl)methyl)piperidin-3-yl)amino)-2-(2,6- dioxopiperidin-3-yl)isoindoline-1,3-dione |
| | 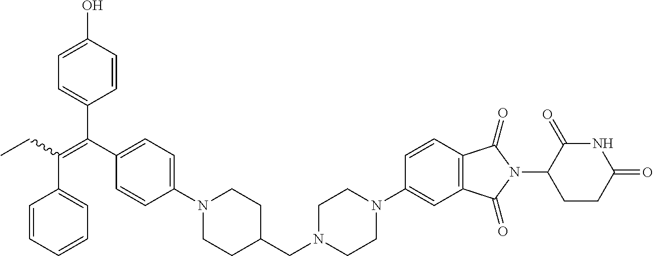 [SMILES: CCC(=C(c1ccc(O)cc1)c1ccc(N2CCC(CN3CCN(c4ccc5c(c4)C(=O)N(C4CCC(=O)NC4=O)C5=O)CC3)CC2)cc1)c1ccccc1] | 2-(2,6-dioxopiperidin-3-yl)-5-(4-((1-(4-(1-(4- hydroxyphenyl)-2-phenylbut-1-en-1- yl)phenyl)piperidin-4-yl)methyl)piperazin-1- yl)isoindoline-1,3-dione |
| | 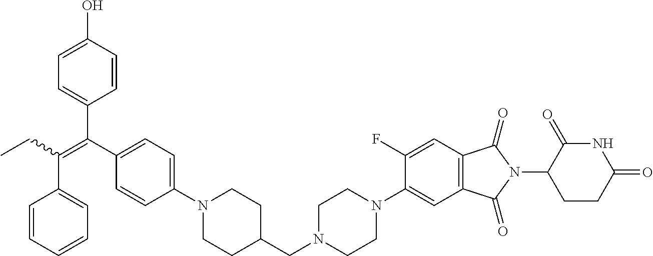 [SMILES: CCC(=C(c1ccc(O)cc1)c1ccc(N2CCC(CN3CCN(c4cc5c(cc4F)C(=O)N(C4CCC(=O)NC4=O)C5=O)CC3)CC2)cc1)c1ccccc1] | 5-(4-((1-(4-(4-chloro-1-(4-hydroxyphenyl)-2- phenylbut-1-en-1-yl)phenyl)piperidin-4- yl)methyl)piperazin-1-yl)-2-(2,6- dioxopiperidin-3-yl)-6-fluoroisoindoline-1,3- dione |
| | 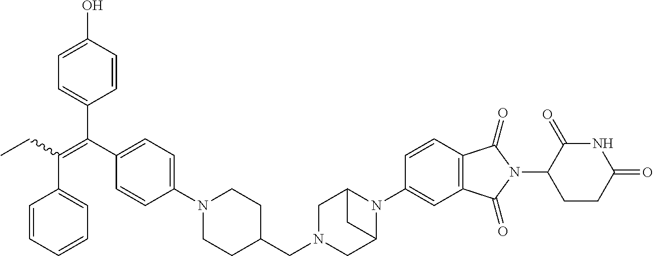 [SMILES: CCC(=C(c1ccc(O)cc1)c1ccc(N2CCC(CN3CC4CC(C3)N4c3ccc4c(c3)C(=O)N(C3CCC(=O)NC3=O)C4=O)CC2)cc1)c1ccccc1] | 2-(2,6-dioxopiperidin-3-yl)-5-(3-((1-(4-(1-(4- hydroxyphenyl)-2-phenylbut-1-en-1- yl)phenyl)piperidin-4-yl)methyl)-3,6- diazabicyclo[3.1.1]heptan-6-yl)isoindoline-1,3- dione |
| | 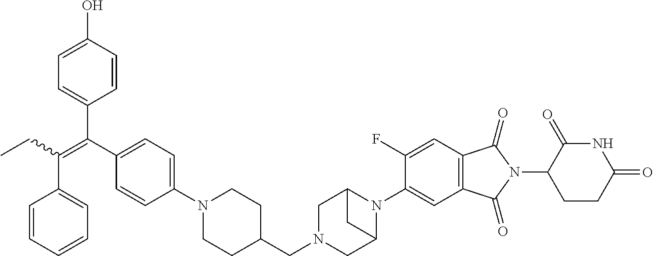 [SMILES: CCC(=C(c1ccc(O)cc1)c1ccc(N2CCC(CN3CC4CC(C3)N4c3cc4c(cc3F)C(=O)N(C3CCC(=O)NC3=O)C4=O)CC2)cc1)c1ccccc1] | 2-(2,6-dioxopiperidin-3-yl)-5-fluoro-6-(3-((1- (4-(1-(4-hydroxyphenyl)-2-phenylbut-1-en-1- yl)phenyl)piperidin-4-yl)methyl)-3,6- diazabicyclo[3.1.1]heptan-6-yl)isoindoline-1,3- dione |
| | 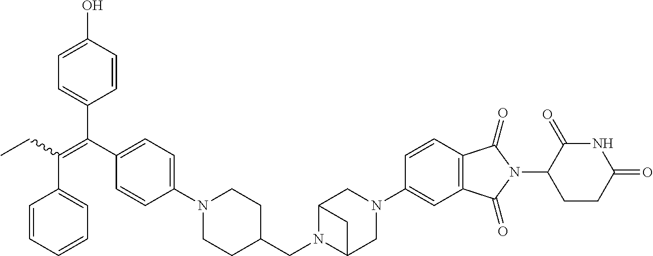 [SMILES: CCC(=C(c1ccc(O)cc1)c1ccc(N2CCC(CN3C4CC3CN(c3ccc5c(c3)C(=O)N(C3CCC(=O)NC3=O)C5=O)C4)CC2)cc1)c1ccccc1] | 2-(2,6-dioxopiperidin-3-yl)-5-(6-((1-(4-(1-(4- hydroxyphenyl)-2-phenylbut-1-en-1- yl)phenyl)piperidin-4-yl)methyl)-3,6- diazabicyclo[3.1.1]heptan-3-yl)isoindoline-1,3- dione |
| | 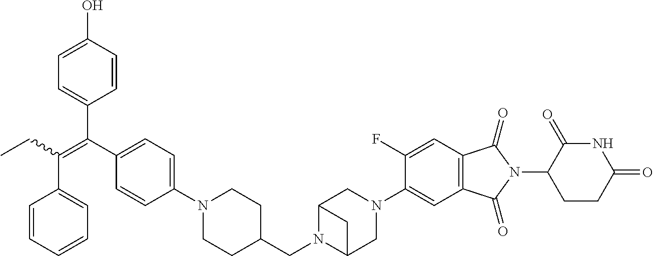 [SMILES: CCC(=C(c1ccc(O)cc1)c1ccc(N2CCC(CN3C4CC3CN(c3cc5c(cc3F)C(=O)N(C3CCC(=O)NC3=O)C5=O)C4)CC2)cc1)c1ccccc1] | 2-(2,6-dioxopiperidin-3-yl)-5-fluoro-6-(6-((1- (4-(1-(4-hydroxyphenyl)-2-phenylbut-1-en-1- yl)phenyl)piperidin-4-yl)methyl)-3,6- diazabicyclo[3.1.1]heptan-3-yl)isoindoline-1,3- dione |
| | 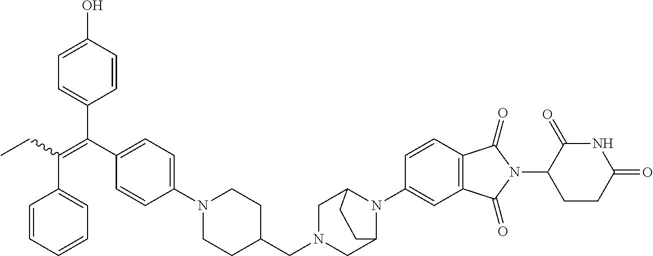 [SMILES: CCC(=C(c1ccc(O)cc1)c1ccc(N2CCC(CN3CC4CCC(C3)N4c3ccc4c(c3)C(=O)N(C3CCC(=O)NC3=O)C4=O)CC2)cc1)c1ccccc1] | 2-(2,6-dioxopiperidin-3-yl)-5-(3-((1-(4-(1-(4- hydroxyphenyl)-2-phenylbut-1-en-1- yl)phenyl)piperidin-4-yl)methyl)-3,8- diazabicyclo[3.2.1]octan-8-yl)isoindoline-1,3- dione |
| | 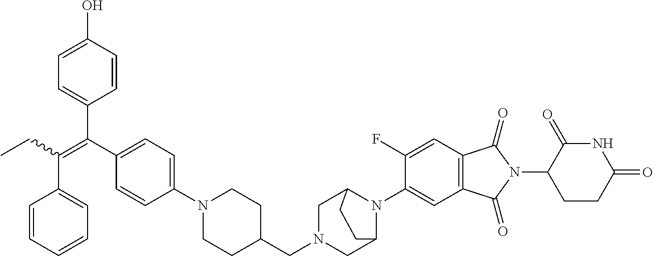 [SMILES: CCC(=C(c1ccc(O)cc1)c1ccc(N2CCC(CN3CC4CCC(C3)N4c3cc4c(cc3F)C(=O)N(C3CCC(=O)NC3=O)C4=O)CC2)cc1)c1ccccc1] | 2-(2,6-dioxopiperidin-3-yl)-5-fluoro-6-(3-((1- (4-(1-(4-hydroxyphenyl)-2-phenylbut-1-en-1- yl)phenyl)piperidin-4-yl)methyl)-3,8- diazabicyclo[3.2.1]octan-8-yl)isoindoline-1,3- dione |
| | 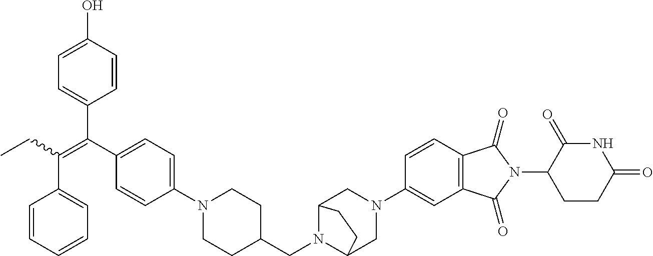 [SMILES: CCC(=C(c1ccc(O)cc1)c1ccc(N2CCC(CN3C4CCC3CN(c3ccc5c(c3)C(=O)N(C3CCC(=O)NC3=O)C5=O)C4)CC2)cc1)c1ccccc1] | 2-(2,6-dioxopiperidin-3-yl)-5-(8-((1-(4-(1-(4- hydroxyphenyl)-2-phenylbut-1-en-1- yl)phenyl)piperidin-4-yl)methyl)-3,8- diazabicyclo[3.2.1]octan-3-yl)isoindoline-1,3- dione |
| | 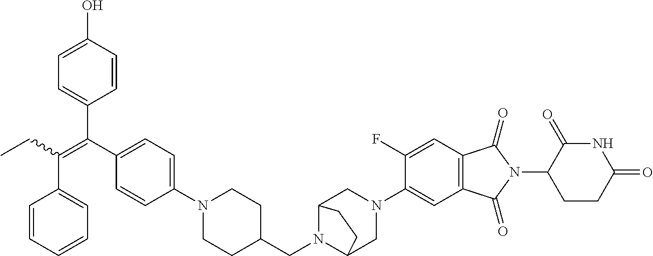 [SMILES: CCC(=C(c1ccc(O)cc1)c1ccc(N2CCC(CN3C4CCC3CN(c3cc5c(cc3F)C(=O)N(C3CCC(=O)NC3=O)C5=O)C4)CC2)cc1)c1ccccc1] | 2-(2,6-dioxopiperidin-3-yl)-5-fluoro-6-(8-((1- (4-(1-(4-hydroxyphenyl)-2-phenylbut-1-en-1- yl)phenyl)piperidin-4-yl)methyl)-3,8- diazabicyclo[3.2.1]octan-3-yl)isoindoline-1,3- dione |
| | 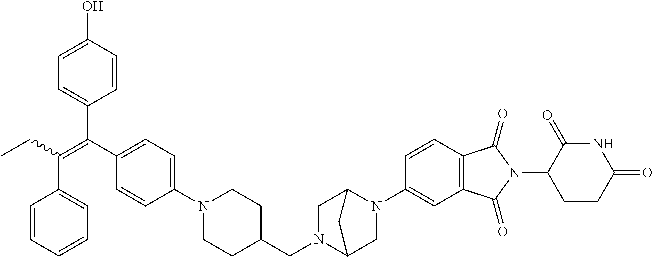 [SMILES: CCC(=C(c1ccc(O)cc1)c1ccc(N2CCC(CN3CC4CC3CN4c3ccc4c(c3)C(=O)N(C3CCC(=O)NC3=O)C4=O)CC2)cc1)c1ccccc1] | 2-(2,6-dioxopiperidin-3-yl)-5-(5-((1-(4-(1-(4- hydroxyphenyl)-2-phenylbut-1-en-1- yl)phenyl)piperidin-4-yl)methyl)-2,5- diazabicyclo[2.2.1]heptan-2-yl)isoindoline-1,3- dione |
| | 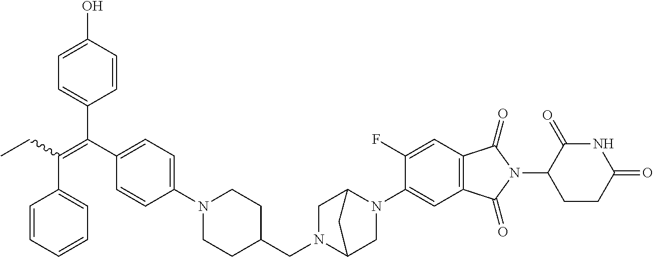 [SMILES: CCC(=C(c1ccc(O)cc1)c1ccc(N2CCC(CN3CC4CC3CN4c3cc4c(cc3F)C(=O)N(C3CCC(=O)NC3=O)C4=O)CC2)cc1)c1ccccc1] | 2-(2,6-dioxopiperidin-3-yl)-5-fluoro-6-(5-((1- (4-(1-(4-hydroxyphenyl)-2-phenylbut-1-en-1- yl)phenyl)piperidin-4-yl)methyl)-2,5- diazabicyclo[2.2.1]heptan-2-yl)isoindoline-1,3- dione |
| | 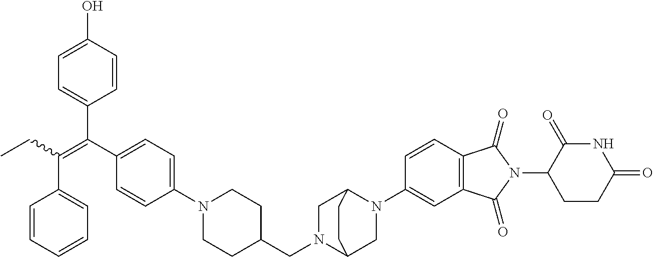 [SMILES: CCC(=C(c1ccc(O)cc1)c1ccc(N2CCC(CN3CC4CCC3CN4c3ccc4c(c3)C(=O)N(C3CCC(=O)NC3=O)C4=O)CC2)cc1)c1ccccc1] | 2-(2,6-dioxopiperidin-3-yl)-5-(5-((1-(4-(1-(4- hydroxyphenyl)-2-phenylbut-1-en-1- yl)phenyl)piperidin-4-yl)methyl)-2,5- diazabicyclo[2.2.2]octan-2-yl)isoindoline-1,3- dione |
| | 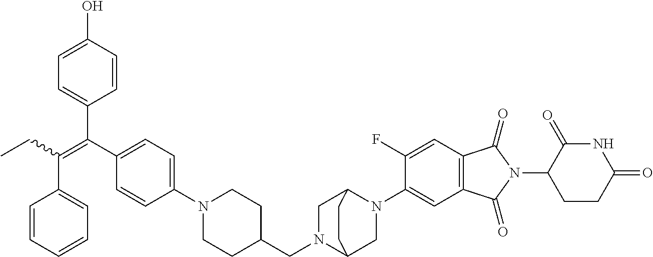 [SMILES: CCC(=C(c1ccc(O)cc1)c1ccc(N2CCC(CN3CC4CCC3CN4c3cc4c(cc3F)C(=O)N(C3CCC(=O)NC3=O)C4=O)CC2)cc1)c1ccccc1] | 2-(2,6-dioxopiperidin-3-yl)-5-fluoro-6-(5-((1- (4-(1-(4-hydroxyphenyl)-2-phenylbut-1-en-1- yl)phenyl)piperidin-4-yl)methyl)-2,5- diazabicyclo[2.2.2]octan-2-yl)isoindoline-1,3- dione |
| | 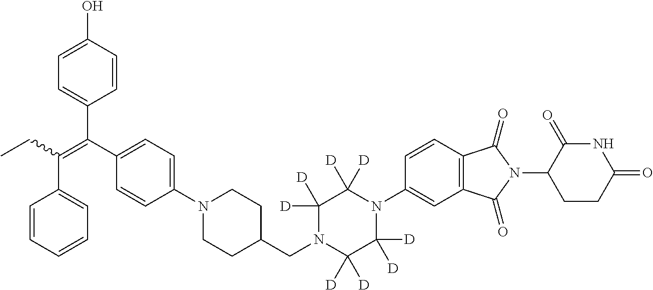 [SMILES: [2H]C1([2H])N(CC2CCN(c3ccc(C(=C(CC)c4ccccc4)c4ccc(O)cc4)cc3)CC2)C([2H])([2H])C([2H])([2H])N(c2ccc3c(c2)C(=O)N(C2CCC(=O)NC2=O)C3=O)C1([2H])[2H]] | 2-(2,6-dioxopiperidin-3-yl)-5-(4-((1-(4-(1-(4- hydroxyphenyl)-2-phenylbut-1-en-1- yl)phenyl)piperidin-4-yl)methyl)piperazin-1-yl- 2,2,3,3,5,5,6,6-d₈)isoindoline-1,3-dione |
| | 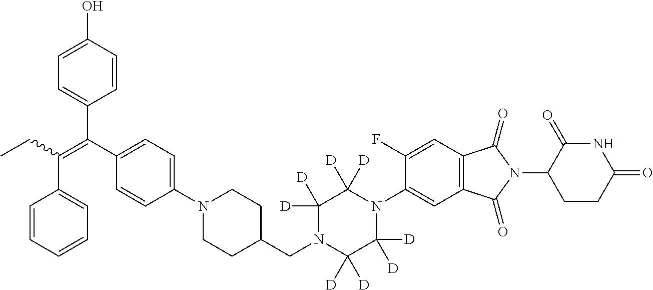 [SMILES: [2H]C1([2H])N(CC2CCN(c3ccc(C(=C(CC)c4ccccc4)c4ccc(O)cc4)cc3)CC2)C([2H])([2H])C([2H])([2H])N(c2cc3c(cc2F)C(=O)N(C2CCC(=O)NC2=O)C3=O)C1([2H])[2H]] | 2-(2,6-dioxopiperidin-3-yl)-5-fluoro-6-(4-((1- (4-(1-(4-hydroxyphenyl)-2-phenylbut-1-en-1- yl)phenyl)piperidin-4-yl)methyl)piperazin-1-yl- 2,2,3,3,5,5,6,6-d₈)isoindoline-1,3-dione |
| | 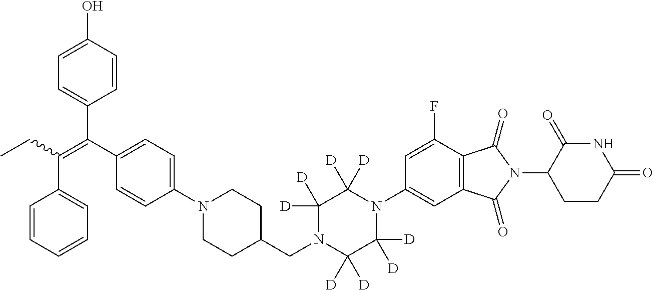 [SMILES: [2H]C1([2H])N(CC2CCN(c3ccc(C(=C(CC)c4ccccc4)c4ccc(O)cc4)cc3)CC2)C([2H])([2H])C([2H])([2H])N(c2cc(F)c3c(c2)C(=O)N(C2CCC(=O)NC2=O)C3=O)C1([2H])[2H]] | 2-(2,6-dioxopiperidin-3-yl)-4-fluoro-6-(4-((1- (4-(1-(4-hydroxyphenyl)-2-phenylbut-1-en-1- yl)phenyl)piperidin-4-yl)methyl)piperazin-1-yl- 2,2,3,3,5,5,6,6-d₈)isoindoline-1,3-dione |
| | 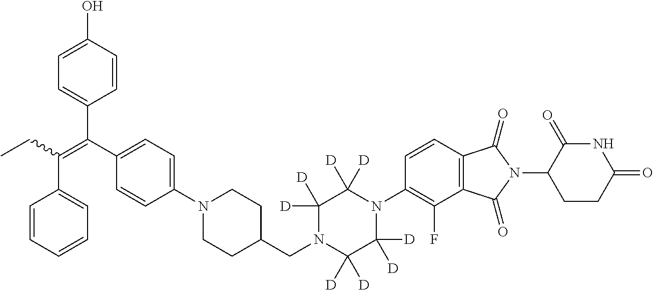 [SMILES: [2H]C1([2H])N(CC2CCN(c3ccc(C(=C(CC)c4ccccc4)c4ccc(O)cc4)cc3)CC2)C([2H])([2H])C([2H])([2H])N(c2ccc3c(c2F)C(=O)N(C2CCC(=O)NC2=O)C3=O)C1([2H])[2H]] | 2-(2,6-dioxopiperidin-3-yl)-4-fluoro-5-(4-((1- (4-(1-(4-hydroxyphenyl)-2-phenylbut-1-en-1- yl)phenyl)piperidin-4-yl)methyl)piperazin-1-yl- 2,2,3,3,5,5,6,6-d₈)isoindoline-1,3-dione |
| | 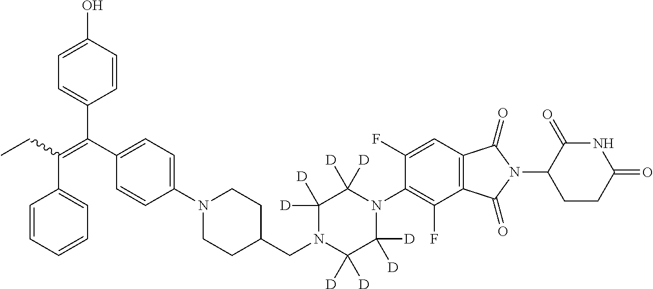 [SMILES: [2H]C1([2H])N(CC2CCN(c3ccc(C(=C(CC)c4ccccc4)c4ccc(O)cc4)cc3)CC2)C([2H])([2H])C([2H])([2H])N(c2c(F)cc3c(c2F)C(=O)N(C2CCC(=O)NC2=O)C3=O)C1([2H])[2H]] | 2-(2,6-dioxopiperidin-3-yl)-4,6-difluoro-5-(4- ((1-(4-(1-(4-hydroxyphenyl)-2-phenylbut-1-en- 1-yl)phenyl)piperidin-4-yl)methyl)piperazin-1- yl-2,2,3,3,5,5,6,6-d₈)isoindoline-1,3-dione |
| | 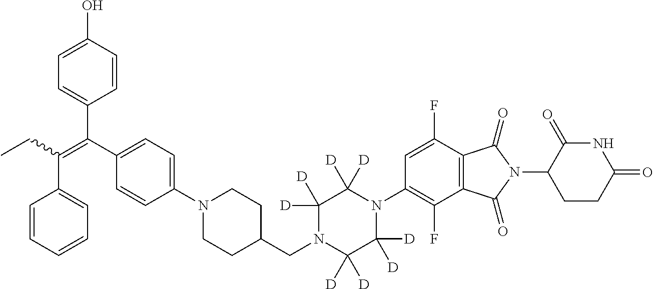 [SMILES: [2H]C1([2H])N(CC2CCN(c3ccc(C(=C(CC)c4ccccc4)c4ccc(O)cc4)cc3)CC2)C([2H])([2H])C([2H])([2H])N(c2cc(F)c3c(c2F)C(=O)N(C2CCC(=O)NC2=O)C3=O)C1([2H])[2H]] | 2-(2,6-dioxopiperidin-3-yl)-4,7-difluoro-5-(4- ((1-(4-(1-(4-hydroxyphenyl)-2-phenylbut-1-en- 1-yl)phenyl)piperidin-4-yl)methyl)piperazin-1- yl-2,2,3,3,5,5,6,6-d₈)isoindoline-1,3-dione |
| | 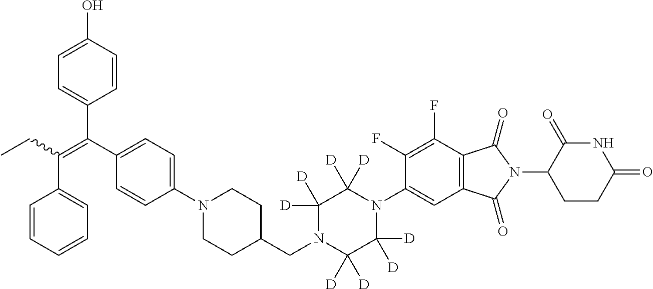 [SMILES: [2H]C1([2H])N(CC2CCN(c3ccc(C(=C(CC)c4ccccc4)c4ccc(O)cc4)cc3)CC2)C([2H])([2H])C([2H])([2H])N(c2cc3c(c(F)c2F)C(=O)N(C2CCC(=O)NC2=O)C3=O)C1([2H])[2H]] | 2-(2,6-dioxopiperidin-3-yl)-4,5-difluoro-6-(4- ((1-(4-(1-(4-hydroxyphenyl)-2-phenylbut-1-en- 1-yl)phenyl)piperidin-4-yl)methyl)piperazin-1- yl-2,2,3,3,5,5,6,6-d₈)isoindoline-1,3-dione |
| | 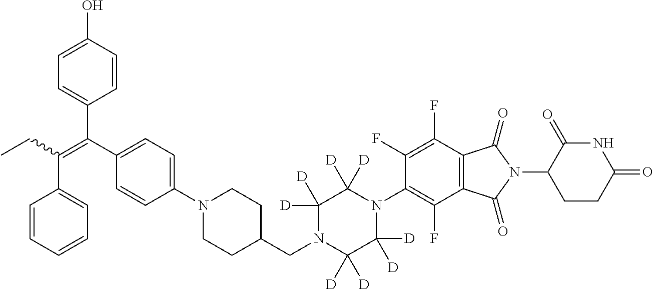 [SMILES: [2H]C1([2H])N(CC2CCN(c3ccc(C(=C(CC)c4ccccc4)c4ccc(O)cc4)cc3)CC2)C([2H])([2H])C([2H])([2H])N(c2c(F)c(F)c3c(c2F)C(=O)N(C2CCC(=O)NC2=O)C3=O)C1([2H])[2H]] | 2-(2,6-dioxopiperidin-3-yl)-4,5,7-trifluoro-6- (4-((1-(4-(1-(4-hydroxyphenyl)-2-phenylbut-1- en-1-yl)phenyl)piperidin-4- yl)methyl)piperazin-1-yl-2,2,3,3,5,5,6,6- d₈)isoindoline-1,3-dione |
| | 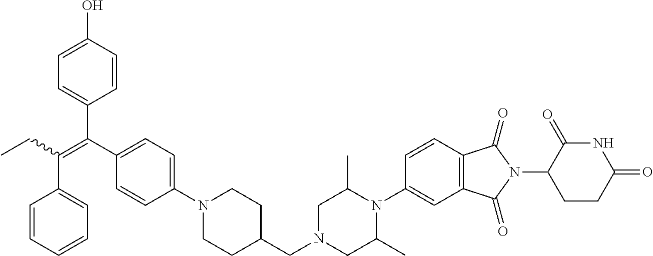 [SMILES: CCC(=C(c1ccc(O)cc1)c1ccc(N2CCC(CN3CC(C)N(c4ccc5c(c4)C(=O)N(C4CCC(=O)NC4=O)C5=O)C(C)C3)CC2)cc1)c1ccccc1] | 2-(2,6-dioxopiperidin-3-yl)-5-(4-((1-(4-(1-(4- hydroxyphenyl)-2-phenylbut-1-en-1- yl)phenyl)piperidin-4-yl)methyl)-2,6- dimethylpiperazin-1-yl)isoindoline-1,3-dione |
| | 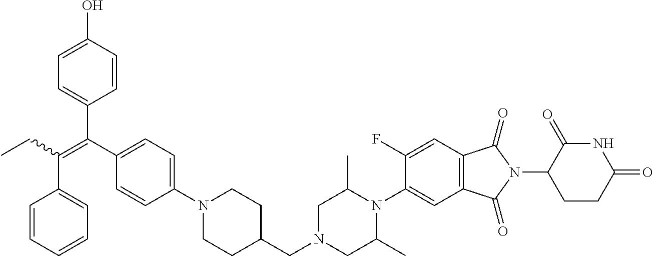 [SMILES: CCC(=C(c1ccc(O)cc1)c1ccc(N2CCC(CN3CC(C)N(c4cc5c(cc4F)C(=O)N(C4CCC(=O)NC4=O)C5=O)C(C)C3)CC2)cc1)c1ccccc1] | 2-(2,6-dioxopiperidin-3-yl)-5-fluoro-6-(4-((1- (4-(1-(4-hydroxyphenyl)-2-phenylbut-1-en-1- yl)phenyl)piperidin-4-yl)methyl)-2,6- dimethylpiperazin-1-yl)isoindoline-1,3-dione |
| | 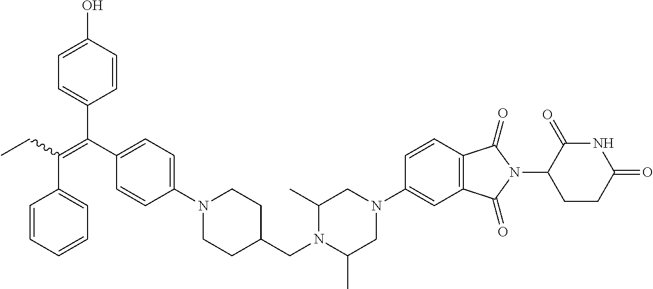 [SMILES: CCC(=C(c1ccc(O)cc1)c1ccc(N2CCC(CN3C(C)CN(c4ccc5c(c4)C(=O)N(C4CCC(=O)NC4=O)C5=O)CC3C)CC2)cc1)c1ccccc1] | 2-(2,6-dioxopiperidin-3-yl)-5-(4-((1-(4-(1-(4- hydroxyphenyl)-2-phenylbut-1-en-1- yl)phenyl)piperidin-4-yl)methyl)-3,5- dimethylpiperazin-1-yl)isoindoline-1,3-dione |
| | 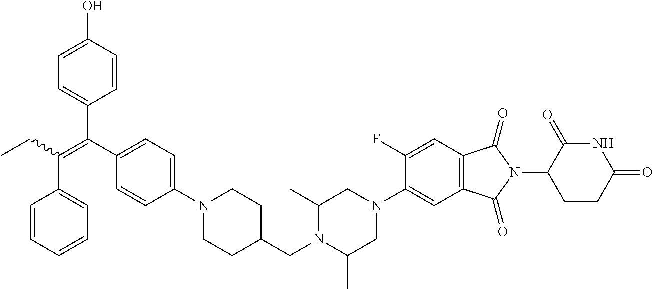 [SMILES: CCC(=C(c1ccc(O)cc1)c1ccc(N2CCC(CN3C(C)CN(c4cc5c(cc4F)C(=O)N(C4CCC(=O)NC4=O)C5=O)CC3C)CC2)cc1)c1ccccc1] | 2-(2,6-dioxopiperidin-3-yl)-5-fluoro-6-(4-((1- (4-(1-(4-hydroxyphenyl)-2-phenylbut-1-en-1- yl)phenyl)piperidin-4-yl)methyl)-3,5- dimethylpiperazin-1-yl)isoindoline-1,3-dione |
| | 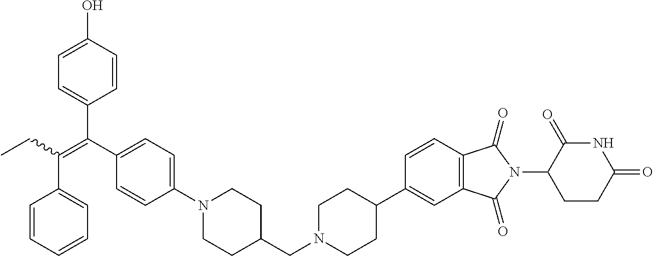 [SMILES: CCC(=C(c1ccc(O)cc1)c1ccc(N2CCC(CN3CCC(c4ccc5c(c4)C(=O)N(C4CCC(=O)NC4=O)C5=O)CC3)CC2)cc1)c1ccccc1] | 2-(2,6-dioxopiperidin-3-yl)-5-(1-((1-(4-(1-(4- hydroxyphenyl)-2-phenylbut-1-en-1- yl)phenyl)piperidin-4-yl)methyl)piperidin-4- yl)isoindoline-1,3-dione |
| | 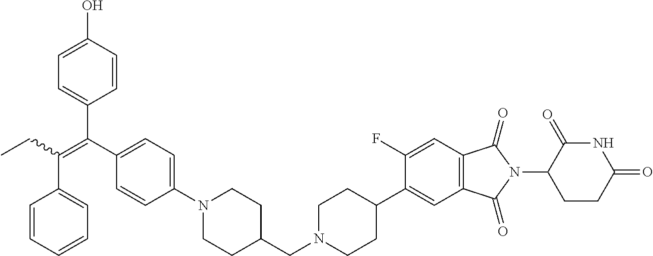 [SMILES: CCC(=C(c1ccc(O)cc1)c1ccc(N2CCC(CN3CCC(c4cc5c(cc4F)C(=O)N(C4CCC(=O)NC4=O)C5=O)CC3)CC2)cc1)c1ccccc1] | 2-(2,6-dioxopiperidin-3-yl)-5-fluoro-6-(1-((1- (4-(1-(4-hydroxyphenyl)-2-phenylbut-1-en-1- yl)phenyl)piperidin-4-yl)methyl)piperidin-4- yl)isoindoline-1,3-dione |
| | 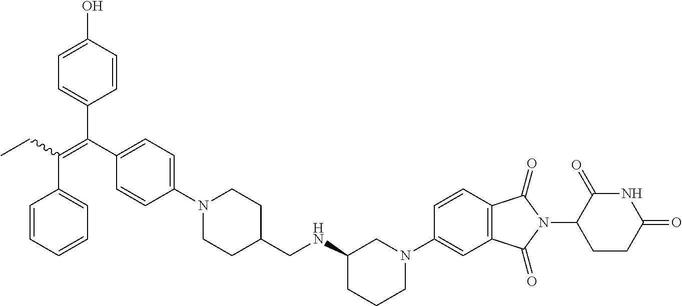 [SMILES: CCC(=C(c1ccc(O)cc1)c1ccc(N2CCC(CN[C@@H]3CCCN(c4ccc5c(c4)C(=O)N(C4CCC(=O)NC4=O)C5=O)C3)CC2)cc1)c1ccccc1] | 2-(2,6-dioxopiperidin-3-yl)-5-((R)-3-(((1-(4-(1- (4-hydroxyphenyl)-2-phenylbut-1-en-1- yl)phenyl)piperidin-4- yl)methyl)amino)piperidin-1-yl)isoindoline- 1,3-dione |
| | 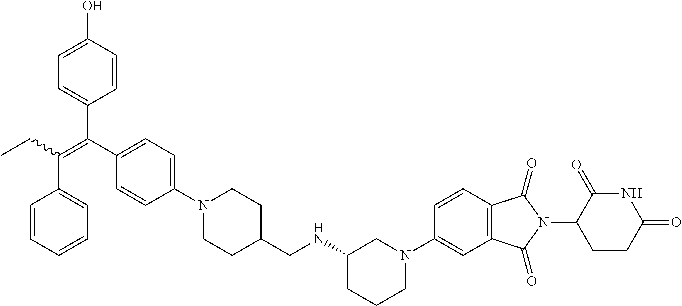 [SMILES: CCC(=C(c1ccc(O)cc1)c1ccc(N2CCC(CN[C@H]3CCCN(c4ccc5c(c4)C(=O)N(C4CCC(=O)NC4=O)C5=O)C3)CC2)cc1)c1ccccc1] | 2-(2,6-dioxopiperidin-3-yl)-5-((S)-3-(((1-(4-(1- (4-hydroxyphenyl)-2-phenylbut-1-en-1- yl)phenyl)piperidin-4- yl)methyl)amino)piperidin-1-yl)isoindoline- 1,3-dione |
| | 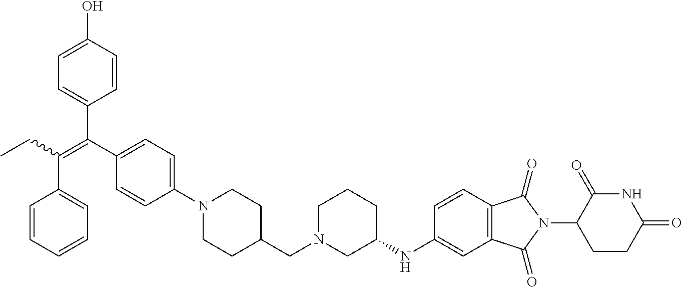 [SMILES: CCC(=C(c1ccc(O)cc1)c1ccc(N2CCC(CN3CCC[C@H](Nc4ccc5c(c4)C(=O)N(C4CCC(=O)NC4=O)C5=O)C3)CC2)cc1)c1ccccc1] | 2-(2,6-dioxopiperidin-3-yl)-5-(((S)-1-((1-(4-(1- (4-hydroxyphenyl)-2-phenylbut-1-en-1- yl)phenyl)piperidin-4-yl)methyl)piperidin-3- yl)amino)isoindoline-1,3-dione |
| | 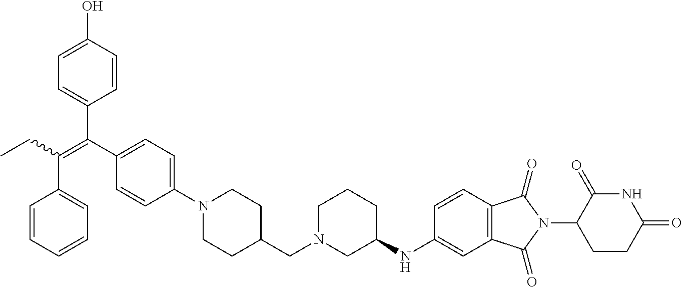 [SMILES: CCC(=C(c1ccc(O)cc1)c1ccc(N2CCC(CN3CCC[C@@H](Nc4ccc5c(c4)C(=O)N(C4CCC(=O)NC4=O)C5=O)C3)CC2)cc1)c1ccccc1] | 2-(2,6-dioxopiperidin-3-yl)-5-(((R)-1-((1-(4-(1- (4-hydroxyphenyl)-2-phenylbut-1-en-1- yl)phenyl)piperidin-4-yl)methyl)piperidin-3- yl)amino)isoindoline-1,3-dione |
| | 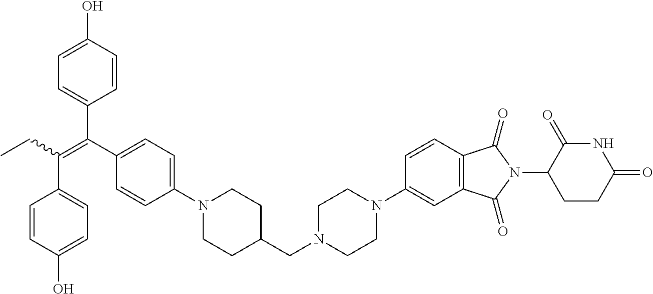 [SMILES: CCC(=C(c1ccc(O)cc1)c1ccc(N2CCC(CN3CCN(c4ccc5c(c4)C(=O)N(C4CCC(=O)NC4=O)C5=O)CC3)CC2)cc1)c1ccc(O)cc1] | 5-(4-((1-(4-(1,2-bis(4-hydroxyphenyl)but-1-en- 1-yl)phenyl)piperidin-4-yl)methyl)piperazin-1- yl)-2-(2,6-dioxopiperidin-3-yl)isoindoline-1,3- dione |
| | 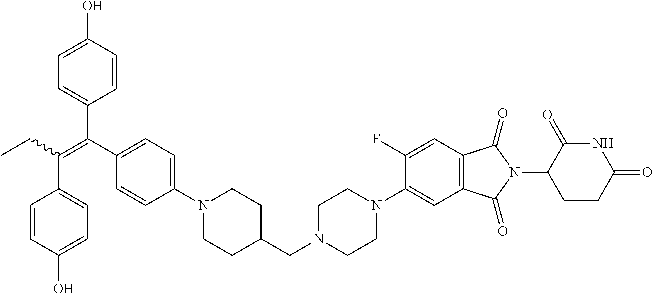 [SMILES: CCC(=C(c1ccc(O)cc1)c1ccc(N2CCC(CN3CCN(c4cc5c(cc4F)C(=O)N(C4CCC(=O)NC4=O)C5=O)CC3)CC2)cc1)c1ccc(O)cc1] | 5-(4-((1-(4-(1,2-bis(4-hydroxyphenyl)but-1-en- 1-yl)phenyl)piperidin-4-yl)methyl)piperazin-1- yl)-2-(2,6-dioxopiperidin-3-yl)-6- fluoroisoindoline-1,3-dione |
| | 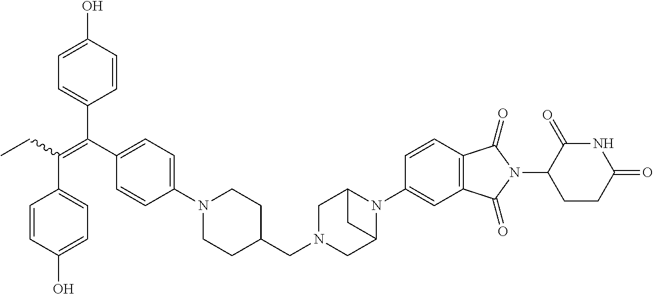 [SMILES: CCC(=C(c1ccc(O)cc1)c1ccc(N2CCC(CN3CC4CC(C3)N4c3ccc4c(c3)C(=O)N(C3CCC(=O)NC3=O)C4=O)CC2)cc1)c1ccc(O)cc1] | 5-(3-((1-(4-(1,2-bis(4-hydroxyphenyl)but-1-en- 1-yl)phenyl)piperidin-4-yl)methyl)-3,6- diazabicyclo[3.1.1]heptan-6-yl)-2-(2,6- dioxopiperidin-3-yl)isoindoline-1,3-dione |
| | 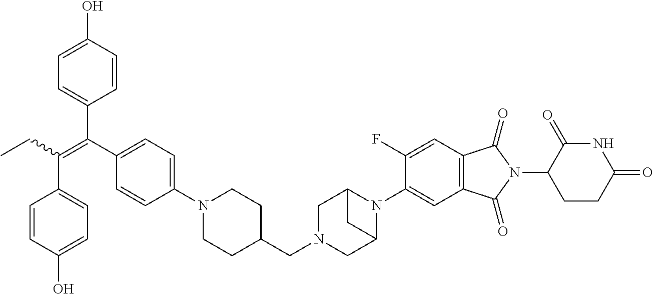 [SMILES: CCC(=C(c1ccc(O)cc1)c1ccc(N2CCC(CN3CC4CC(C3)N4c3cc4c(cc3F)C(=O)N(C3CCC(=O)NC3=O)C4=O)CC2)cc1)c1ccc(O)cc1] | 5-(3-((1-(4-(1,2-bis(4-hydroxyphenyl)but-1-en- 1-yl)phenyl)piperidin-4-yl)methyl)-3,6- diazabicyclo[3.1.1]heptan-6-yl)-2-(2,6- dioxopiperidin-3-yl)-6-fluoroisoindoline-1,3- dione |
| | 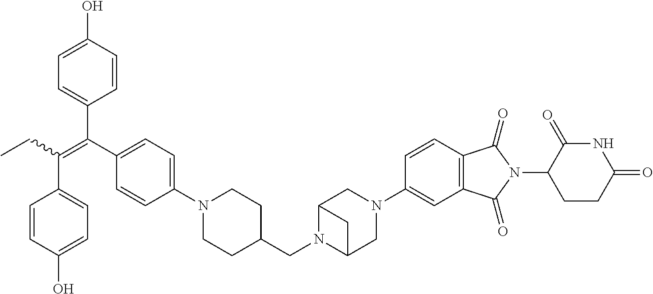 [SMILES: CCC(=C(c1ccc(O)cc1)c1ccc(N2CCC(CN3C4CC3CN(c3ccc5c(c3)C(=O)N(C3CCC(=O)NC3=O)C5=O)C4)CC2)cc1)c1ccc(O)cc1] | 5-(6-((1-(4-(1,2-bis(4-hydroxyphenyl)but-1-en- 1-yl)phenyl)piperidin-4-yl)methyl)-3,6- diazabicyclo[3.1.1]heptan-3-yl)-2-(2,6- dioxopiperidin-3-yl)isoindoline-1,3-dione |
| | 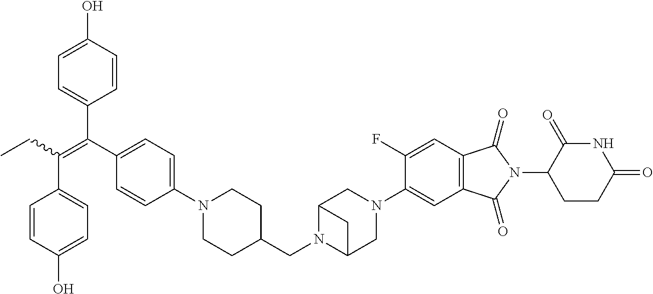 [SMILES: CCC(=C(c1ccc(O)cc1)c1ccc(N2CCC(CN3C4CC3CN(c3cc5c(cc3F)C(=O)N(C3CCC(=O)NC3=O)C5=O)C4)CC2)cc1)c1ccc(O)cc1] | 5-(6-((1-(4-(1,2-bis(4-hydroxyphenyl)but-1-en- 1-yl)phenyl)piperidin-4-yl)methyl)-3,6- diazabicyclo[3.1.1]heptan-3-yl)-2-(2,6- dioxopiperidin-3-yl)-6-fluoroisoindoline-1,3- dione |
| | 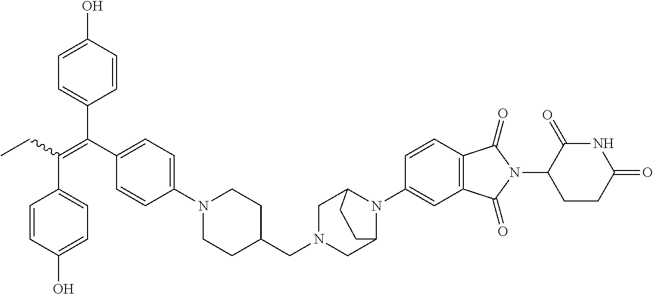 [SMILES: CCC(=C(c1ccc(O)cc1)c1ccc(N2CCC(CN3CC4CCC(C3)N4c3ccc4c(c3)C(=O)N(C3CCC(=O)NC3=O)C4=O)CC2)cc1)c1ccc(O)cc1] | 5-(3-((1-(4-(1,2-bis(4-hydroxyphenyl)but-1-en- 1-yl)phenyl)piperidin-4-yl)methyl)-3,8- diazabicyclo[3.2.1]octan-8-yl)-2-(2,6- dioxopiperidin-3-yl)isoindoline-1,3-dione |
| | 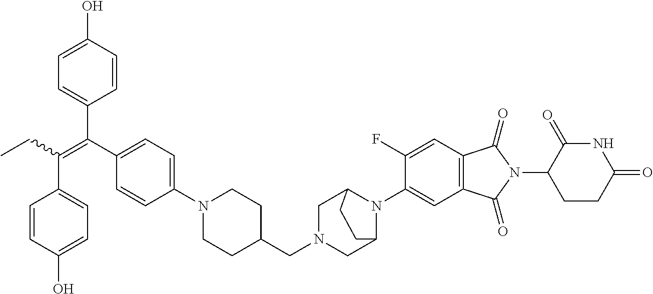 [SMILES: CCC(=C(c1ccc(O)cc1)c1ccc(N2CCC(CN3CC4CCC(C3)N4c3cc4c(cc3F)C(=O)N(C3CCC(=O)NC3=O)C4=O)CC2)cc1)c1ccc(O)cc1] | 5-(3-((1-(4-(1,2-bis(4-hydroxyphenyl)but-1-en- 1-yl)phenyl)piperidin-4-yl)methyl)-3,8- diazabicyclo[3.2.1]octan-8-yl)-2-(2,6- dioxopiperidin-3-yl)-6-fluoroisoindoline-1,3- dione |
| | 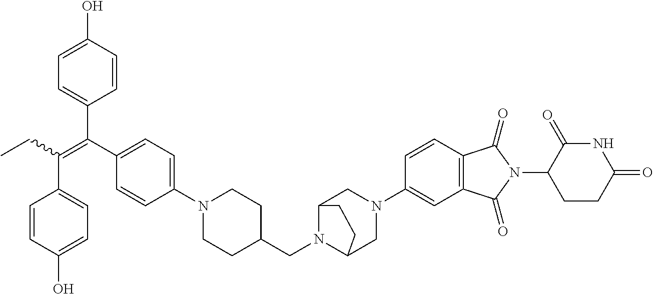 [SMILES: CCC(=C(c1ccc(O)cc1)c1ccc(N2CCC(CN3C4CCC3CN(c3ccc5c(c3)C(=O)N(C3CCC(=O)NC3=O)C5=O)C4)CC2)cc1)c1ccc(O)cc1] | 5-(8-((1-(4-(1,2-bis(4-hydroxyphenyl)but-1-en- 1-yl)phenyl)piperidin-4-yl)methyl)-3,8- diazabicyclo[3.2.1]octan-3-yl)-2-(2,6- dioxopiperidin-3-yl)isoindoline-1,3-dione |
| | 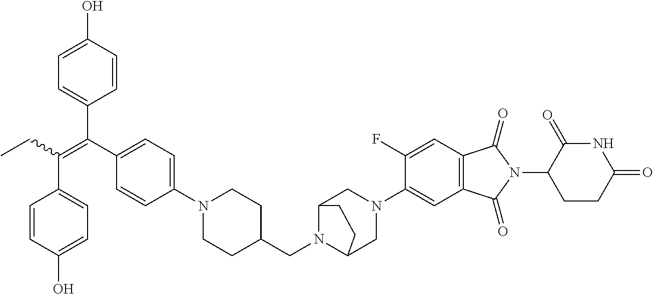 [SMILES: CCC(=C(c1ccc(O)cc1)c1ccc(N2CCC(CN3C4CCC3CN(c3cc5c(cc3F)C(=O)N(C3CCC(=O)NC3=O)C5=O)C4)CC2)cc1)c1ccc(O)cc1] | 5-(8-((1-(4-(1,2-bis(4-hydroxyphenyl)but-1-en- 1-yl)phenyl)piperidin-4-yl)methyl)-3,8- diazabicyclo[3.2.1]octan-3-yl)-2-(2,6- dioxopiperidin-3-yl)-6-fluoroisoindoline-1,3- dione |
| | 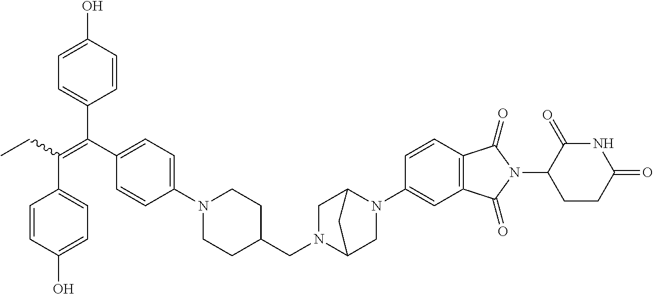 [SMILES: CCC(=C(c1ccc(O)cc1)c1ccc(N2CCC(CN3CC4CC3CN4c3ccc4c(c3)C(=O)N(C3CCC(=O)NC3=O)C4=O)CC2)cc1)c1ccc(O)cc1] | 5-(5-((1-(4-(1,2-bis(4-hydroxyphenyl)but-1-en- 1-yl)phenyl)piperidin-4-yl)methyl)-2,5- diazabicyclo[2.2.1]heptan-2-yl)-2-(2,6- dioxopiperidin-3-yl)isoindoline-1,3-dione |
| | 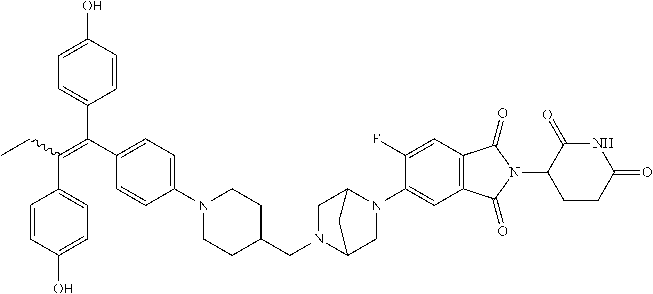 [SMILES: CCC(=C(c1ccc(O)cc1)c1ccc(N2CCC(CN3CC4CC3CN4c3cc4c(cc3F)C(=O)N(C3CCC(=O)NC3=O)C4=O)CC2)cc1)c1ccc(O)cc1] | 5-(5-((1-(4-(1,2-bis(4-hydroxyphenyl)but-1-en- 1-yl)phenyl)piperidin-4-yl)methyl)-2,5- diazabicyclo[2.2.1]heptan-2-yl)-2-(2,6- dioxopiperidin-3-yl)-6-fluoroisoindoline-1,3- dione |
| | 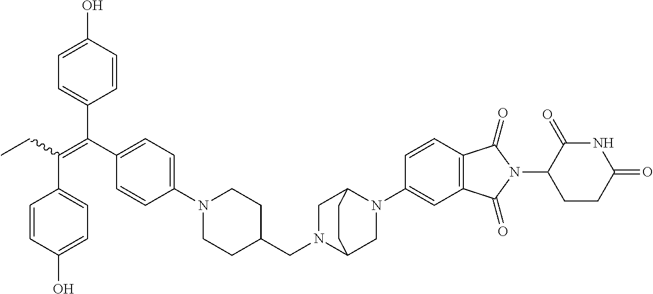 [SMILES: CCC(=C(c1ccc(O)cc1)c1ccc(N2CCC(CN3CC4CCC3CN4c3ccc4c(c3)C(=O)N(C3CCC(=O)NC3=O)C4=O)CC2)cc1)c1ccc(O)cc1] | 5-(5-((1-(4-(1,2-bis(4-hydroxyphenyl)but-1-en- 1-yl)phenyl)piperidin-4-yl)methyl)-2,5- diazabicyclo[2.2.2]octan-2-yl)-2-(2,6- dioxopiperidin-3-yl)isoindoline-1,3-dione |
| | 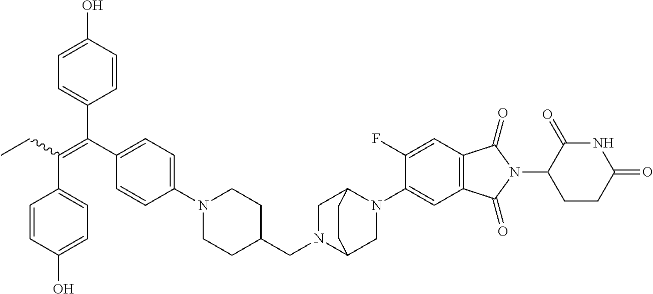 [SMILES: CCC(=C(c1ccc(O)cc1)c1ccc(N2CCC(CN3CC4CCC3CN4c3cc4c(cc3F)C(=O)N(C3CCC(=O)NC3=O)C4=O)CC2)cc1)c1ccc(O)cc1] | 5-(5-((1-(4-(1,2-bis(4-hydroxyphenyl)but-1-en- 1-yl)phenyl)piperidin-4-yl)methyl)-2,5- diazabicyclo[2.2.2]octan-2-yl)-2-(2,6- dioxopiperidin-3-yl)-6-fluoroisoindoline-1,3- dione |
| | 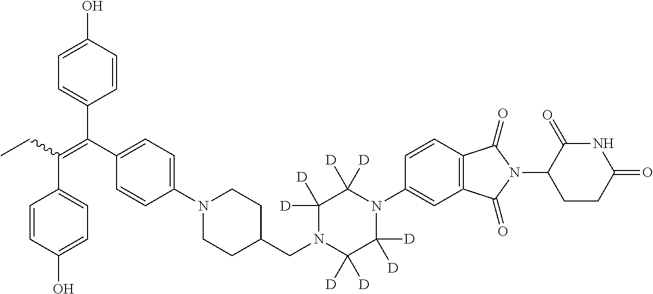 [SMILES: [2H]C1([2H])N(CC2CCN(c3ccc(C(=C(CC)c4ccc(O)cc4)c4ccc(O)cc4)cc3)CC2)C([2H])([2H])C([2H])([2H])N(c2ccc3c(c2)C(=O)N(C2CCC(=O)NC2=O)C3=O)C1([2H])[2H]] | 5-(4-((1-(4-(1,2-bis(4-hydroxyphenyl)but-1-en- 1-yl)phenyl)piperidin-4-yl)methyl)piperazin-1- yl-2,2,3,3,5,5,6,6-d₈)-2-(2,6-dioxopiperidin-3- yl)isoindoline-1,3-dione |
| | 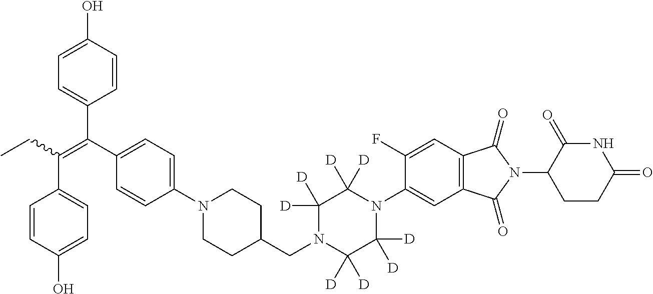 [SMILES: [2H]C1([2H])N(CC2CCN(c3ccc(C(=C(CC)c4ccc(O)cc4)c4ccc(O)cc4)cc3)CC2)C([2H])([2H])C([2H])([2H])N(c2cc3c(cc2F)C(=O)N(C2CCC(=O)NC2=O)C3=O)C1([2H])[2H]] | 5-(4-((1-(4-(1,2-bis(4-hydroxyphenyl)but-1-en- 1-yl)phenyl)piperidin-4-yl)methyl)piperazin-1- yl-2,2,3,3,5,5,6,6-d₈)-2-(2,6-dioxopiperidin-3- yl)-6-fluoroisoindoline-1,3-dione |
| | 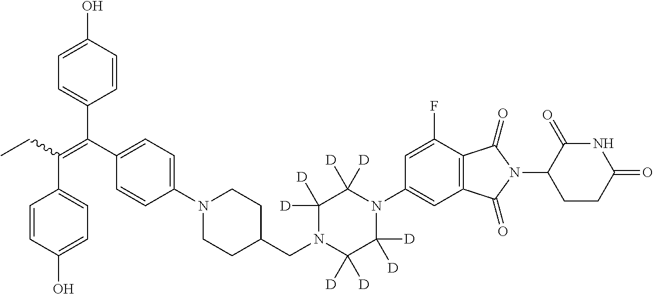 [SMILES: [2H]C1([2H])N(CC2CCN(c3ccc(C(=C(CC)c4ccc(O)cc4)c4ccc(O)cc4)cc3)CC2)C([2H])([2H])C([2H])([2H])N(c2cc(F)c3c(c2)C(=O)N(C2CCC(=O)NC2=O)C3=O)C1([2H])[2H]] | 6-(4-((1-(4-(1,2-bis(4-hydroxyphenyl)but-1-en- 1-yl)phenyl)piperidin-4-yl)methyl)piperazin-1- yl-2,2,3,3,5,5,6,6-d₈)-2-(2,6-dioxopiperidin-3- yl)-4-fluoroisoindoline-1,3-dione |
| | 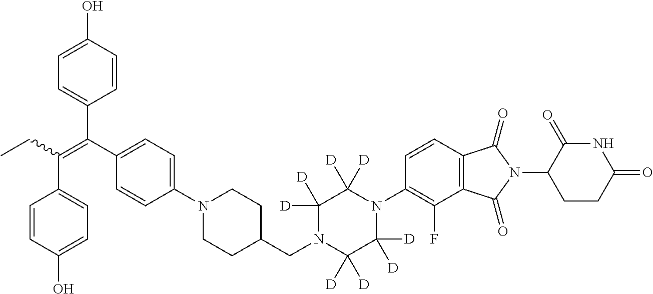 [SMILES: [2H]C1([2H])N(CC2CCN(c3ccc(C(=C(CC)c4ccc(O)cc4)c4ccc(O)cc4)cc3)CC2)C([2H])([2H])C([2H])([2H])N(c2ccc3c(c2F)C(=O)N(C2CCC(=O)NC2=O)C3=O)C1([2H])[2H]] | 5-(4-((1-(4-(1,2-bis(4-hydroxyphenyl)but-1-en- 1-yl)phenyl)piperidin-4-yl)methyl)piperazin-1- yl-2,2,3,3,5,5,6,6-d₈)-2-(2,6-dioxopiperidin-3- yl)-4-fluoroisoindoline-1,3-dione |
| | 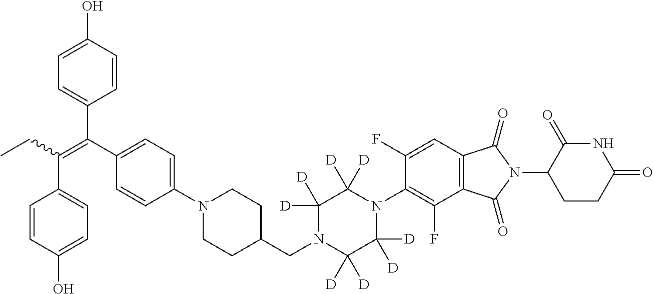 [SMILES: [2H]C1([2H])N(CC2CCN(c3ccc(C(=C(CC)c4ccc(O)cc4)c4ccc(O)cc4)cc3)CC2)C([2H])([2H])C([2H])([2H])N(c2c(F)cc3c(c2F)C(=O)N(C2CCC(=O)NC2=O)C3=O)C1([2H])[2H]] | 5-(4-((1-(4-(1,2-bis(4-hydroxyphenyl)but-1-en- 1-yl)phenyl)piperidin-4-yl)methyl)piperazin-1- yl-2,2,3,3,5,5,6,6-d₈)-2-(2,6-dioxopiperidin-3- yl)-4,6-difluoroisoindoline-1,3-dione |
| | 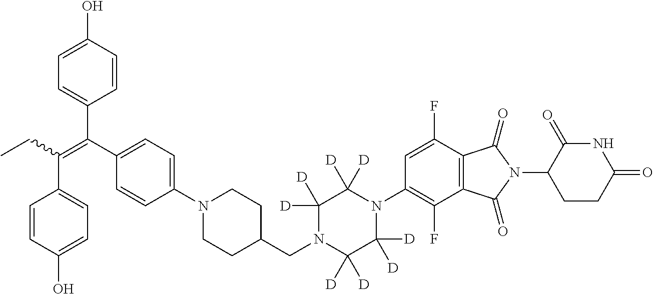 [SMILES: [2H]C1([2H])N(CC2CCN(c3ccc(C(=C(CC)c4ccc(O)cc4)c4ccc(O)cc4)cc3)CC2)C([2H])([2H])C([2H])([2H])N(c2cc(F)c3c(c2F)C(=O)N(C2CCC(=O)NC2=O)C3=O)C1([2H])[2H]] | 5-(4-((1-(4-(1,2-bis(4-hydroxyphenyl)but-1-en- 1-yl)phenyl)piperidin-4-yl)methyl)piperazin-1- yl-2,2,3,3,5,5,6,6-d₈)-2-(2,6-dioxopiperidin-3- yl)-4,7-difluoroisoindoline-1,3-dione |
| | 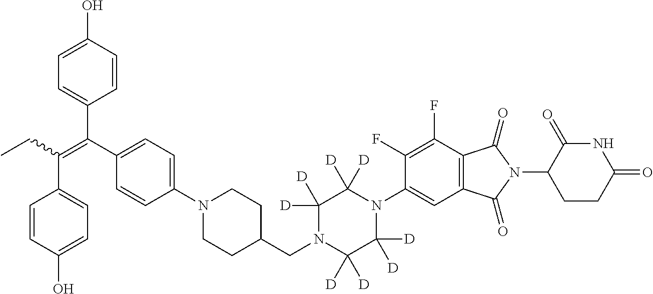 [SMILES: [2H]C1([2H])N(CC2CCN(c3ccc(C(=C(CC)c4ccc(O)cc4)c4ccc(O)cc4)cc3)CC2)C([2H])([2H])C([2H])([2H])N(c2cc3c(c(F)c2F)C(=O)N(C2CCC(=O)NC2=O)C3=O)C1([2H])[2H]] | 6-(4-((1-(4-(1,2-bis(4-hydroxyphenyl)but-1-en- 1-yl)phenyl)piperidin-4-yl)methyl)piperazin-1- yl-2,2,3,3,5,5,6,6-d₈)-2-(2,6-dioxopiperidin-3- yl)-4,5-difluoroisoindoline-1,3-dione |
| | 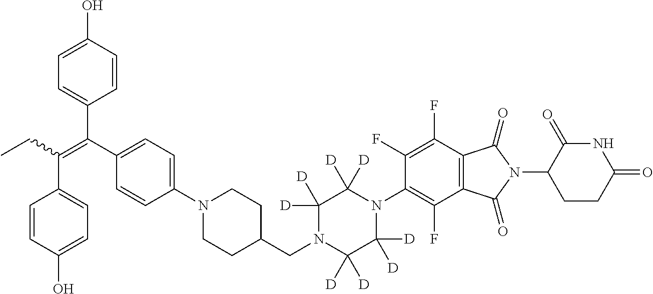 [SMILES: [2H]C1([2H])N(CC2CCN(c3ccc(C(=C(CC)c4ccc(O)cc4)c4ccc(O)cc4)cc3)CC2)C([2H])([2H])C([2H])([2H])N(c2c(F)c(F)c3c(c2F)C(=O)N(C2CCC(=O)NC2=O)C3=O)C1([2H])[2H]] | 5-(4-((1-(4-(1,2-bis(4-hydroxyphenyl)but-1-en- 1-yl)phenyl)piperidin-4-yl)methyl)piperazin-1- yl-2,2,3,3,5,5,6,6-d₈)-2-(2,6-dioxopiperidin-3- yl)-4,6,7-trifluoroisoindoline-1,3-dione |
| | 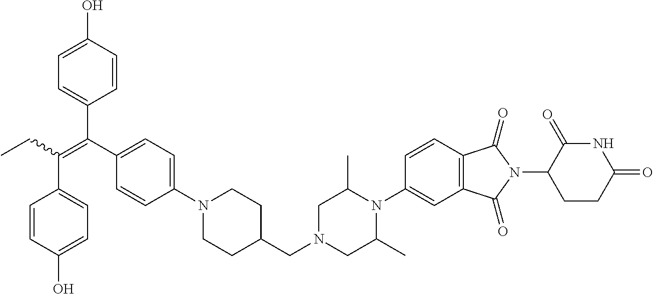 [SMILES: CCC(=C(c1ccc(O)cc1)c1ccc(N2CCC(CN3CC(C)N(c4ccc5c(c4)C(=O)N(C4CCC(=O)NC4=O)C5=O)C(C)C3)CC2)cc1)c1ccc(O)cc1] | 5-(4-((1-(4-(1,2-bis(4-hydroxyphenyl)but-1-en- 1-yl)phenyl)piperidin-4-yl)methyl)-2,6- dimethylpiperazin-1-yl)-2-(2,6-dioxopiperidin- 3-yl)isoindoline-1,3-dione |
| | 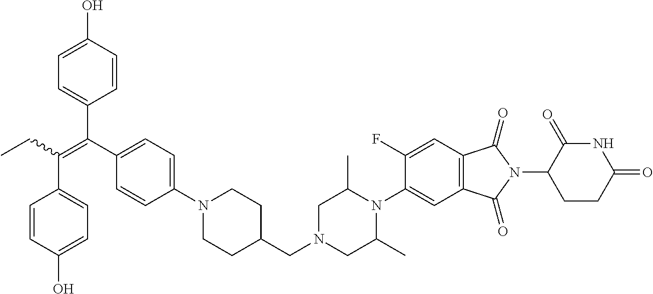 [SMILES: CCC(=C(c1ccc(O)cc1)c1ccc(N2CCC(CN3CC(C)N(c4cc5c(cc4F)C(=O)N(C4CCC(=O)NC4=O)C5=O)C(C)C3)CC2)cc1)c1ccc(O)cc1] | 5-(4-((1-(4-(1,2-bis(4-hydroxyphenyl)but-1-en- 1-yl)phenyl)piperidin-4-yl)methyl)-2,6- dimethylpiperazin-1-yl)-2-(2,6-dioxopiperidin- 3-yl)-6-fluoroisoindoline-1,3-dione |
| | 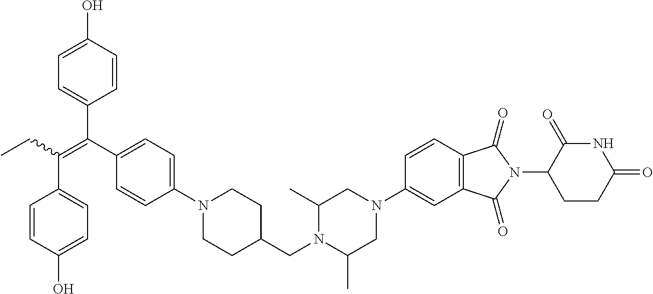 [SMILES: CCC(=C(c1ccc(O)cc1)c1ccc(N2CCC(CN3C(C)CN(c4ccc5c(c4)C(=O)N(C4CCC(=O)NC4=O)C5=O)CC3C)CC2)cc1)c1ccc(O)cc1] | 5-(4-((1-(4-(1,2-bis(4-hydroxyphenyl)but-1-en- 1-yl)phenyl)piperidin-4-yl)methyl)-3,5- dimethylpiperazin-1-yl)-2-(2,6-dioxopiperidin- 3-yl)isoindoline-1,3-dione |
| | 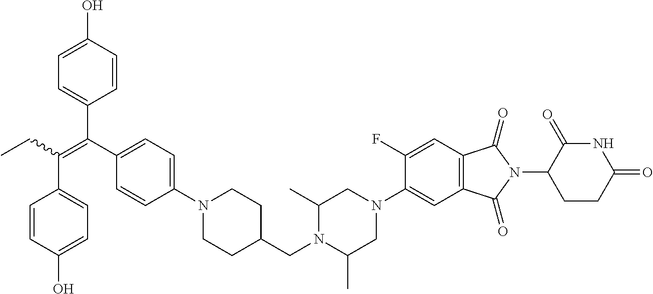 [SMILES: CCC(=C(c1ccc(O)cc1)c1ccc(N2CCC(CN3C(C)CN(c4cc5c(cc4F)C(=O)N(C4CCC(=O)NC4=O)C5=O)CC3C)CC2)cc1)c1ccc(O)cc1] | 5-(4-((1-(4-(1,2-bis(4-hydroxyphenyl)but-1-en- 1-yl)phenyl)piperidin-4-yl)methyl)-3,5- dimethylpiperazin-1-yl)-2-(2,6-dioxopiperidin- 3-yl)-6-fluoroisoindoline-1,3-dione |
| | 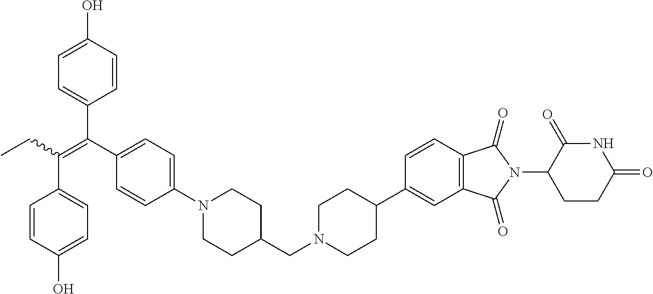 [SMILES: CCC(=C(c1ccc(O)cc1)c1ccc(N2CCC(CN3CCC(c4ccc5c(c4)C(=O)N(C4CCC(=O)NC4=O)C5=O)CC3)CC2)cc1)c1ccc(O)cc1] | 5-(1-((1-(4-(1,2-bis(4-hydroxyphenyl)but-1-en- 1-yl)phenyl)piperidin-4-yl)methyl)piperidin-4- yl)-2-(2,6-dioxopiperidin-3-yl)isoindoline-1,3- dione |
| | 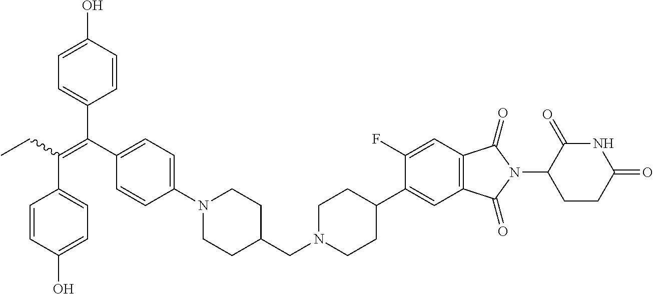 [SMILES: CCC(=C(c1ccc(O)cc1)c1ccc(N2CCC(CN3CCC(c4cc5c(cc4F)C(=O)N(C4CCC(=O)NC4=O)C5=O)CC3)CC2)cc1)c1ccc(O)cc1] | 5-(1-((1-(4-(1,2-bis(4-hydroxyphenyl)but-1-en- 1-yl)phenyl)piperidin-4-yl)methyl)piperidin-4- yl)-2-(2,6-dioxopiperidin-3-yl)-6- fluoroisoindoline-1,3-dione |
| | 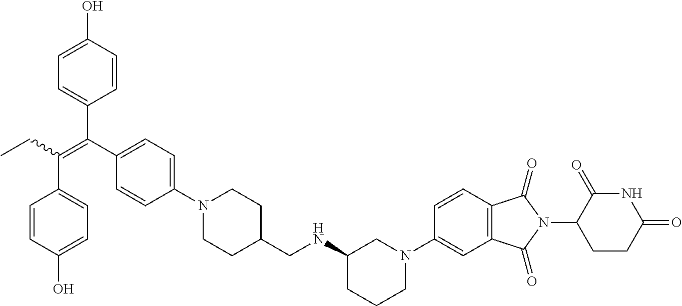 [SMILES: CCC(=C(c1ccc(O)cc1)c1ccc(N2CCC(CN[C@@H]3CCCN(c4ccc5c(c4)C(=O)N(C4CCC(=O)NC4=O)C5=O)C3)CC2)cc1)c1ccc(O)cc1] | 5-((R)-3-(((1-(4-(1,2-bis(4-hydroxyphenyl)but- 1-en-1-yl)phenyl)piperidin-4- yl)methyl)amino)piperidin-1-yl)-2-(2,6- dioxopiperidin-3-yl)isoindoline-1,3-dione |
| | 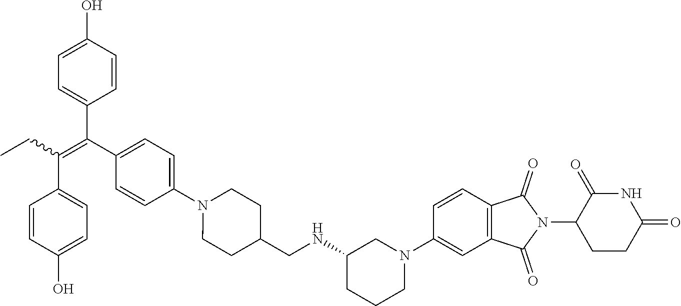 [SMILES: CCC(=C(c1ccc(O)cc1)c1ccc(N2CCC(CN[C@H]3CCCN(c4ccc5c(c4)C(=O)N(C4CCC(=O)NC4=O)C5=O)C3)CC2)cc1)c1ccc(O)cc1] | 5-((S)-3-(((1-(4-(1,2-bis(4-hydroxyphenyl)but- 1-en-1-yl)phenyl)piperidin-4- yl)methyl)amino)piperidin-1-yl)-2-(2,6- dioxopiperidin-3-yl)isoindoline-1,3-dione |
| | 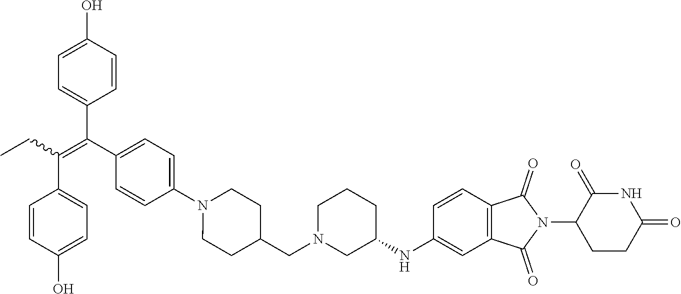 [SMILES: CCC(=C(c1ccc(O)cc1)c1ccc(N2CCC(CN3CCC[C@H](Nc4ccc5c(c4)C(=O)N(C4CCC(=O)NC4=O)C5=O)C3)CC2)cc1)c1ccc(O)cc1] | 5-(((S)-1-((1-(4-(1,2-bis(4-hydroxyphenyl)but- 1-en-1-yl)phenyl)piperidin-4- yl)methyl)piperidin-3-yl)amino)-2-(2,6- dioxopiperidin-3-yl)isoindoline-1,3-dione |
| | 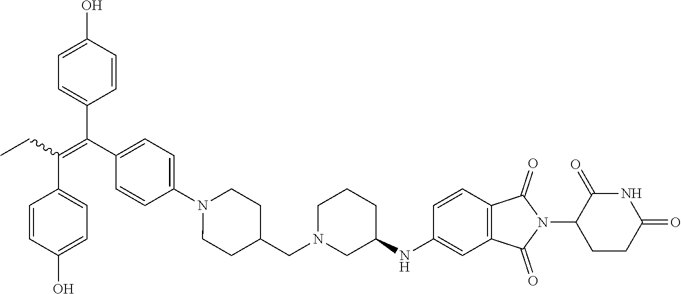 [SMILES: CCC(=C(c1ccc(O)cc1)c1ccc(N2CCC(CN3CCC[C@@H](Nc4ccc5c(c4)C(=O)N(C4CCC(=O)NC4=O)C5=O)C3)CC2)cc1)c1ccc(O)cc1] | 5-(((R)-1-((1-(4-(1,2-bis(4-hydroxyphenyl)but- 1-en-1-yl)phenyl)piperidin-4- yl)methyl)piperidin-3-yl)amino)-2-(2,6- dioxopiperidin-3-yl)isoindoline-1,3-dione |
| | 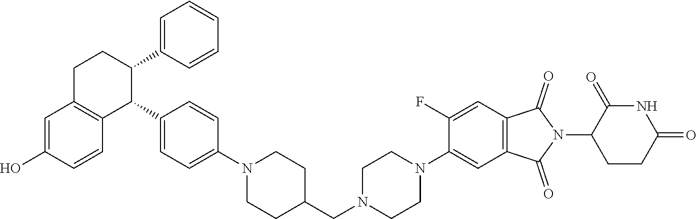 [SMILES: O=C1CCC(N2C(=O)c3cc(F)c(N4CCN(CC5CCN(c6ccc([C@@H]7c8ccc(O)cc8CC[C@@H]7c7ccccc7)cc6)CC5)CC4)cc3C2=O)C(=O)N1] | 2-(2,6-dioxopiperidin-3-yl)-5-fluoro-6-(4-((1- (4-((1R,2S)-6-hydroxy-2-phenyl-1,2,3,4- tetrahydronaphthalen-1-yl)phenyl)piperidin-4- yl)methyl)piperazin-1-yl)isoindoline-1,3-dione |
| | 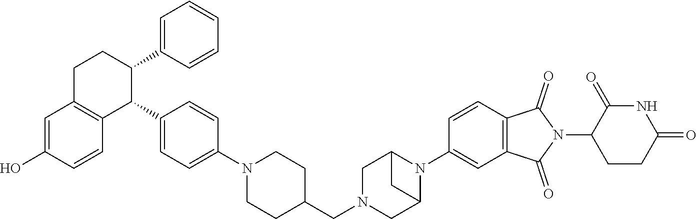 [SMILES: O=C1CCC(N2C(=O)c3ccc(N4C5CC4CN(CC4CCN(c6ccc([C@@H]7c8ccc(O)cc8CC[C@@H]7c7ccccc7)cc6)CC4)C5)cc3C2=O)C(=O)N1] | 2-(2,6-dioxopiperidin-3-yl)-5-(3-((1-(4- ((1R,2S)-6-hydroxy-2-phenyl-1,2,3,4- tetrahydronaphthalen-1-yl)phenyl)piperidin-4- yl)methyl)-3,6-diazabicyclo[3.1.1]heptan-6- yl)isoindoline-1,3-dione |
| | 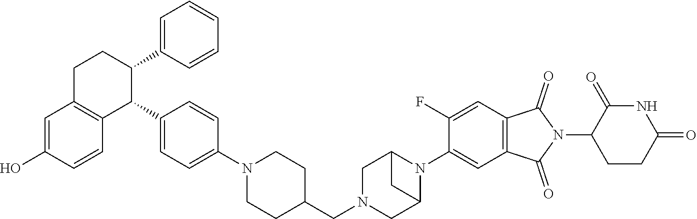 [SMILES: O=C1CCC(N2C(=O)c3cc(F)c(N4C5CC4CN(CC4CCN(c6ccc([C@@H]7c8ccc(O)cc8CC[C@@H]7c7ccccc7)cc6)CC4)C5)cc3C2=O)C(=O)N1] | 2-(2,6-dioxopiperidin-3-yl)-5-fluoro-6-(3-((1- (4-((1R,2S)-6-hydroxy-2-phenyl-1,2,3,4- tetrahydronaphthalen-1-yl)phenyl)piperidin-4- yl)methyl)-3,6-diazabicyclo[3.1.1]heptan-6- yl)isoindoline-1,3-dione |
| | 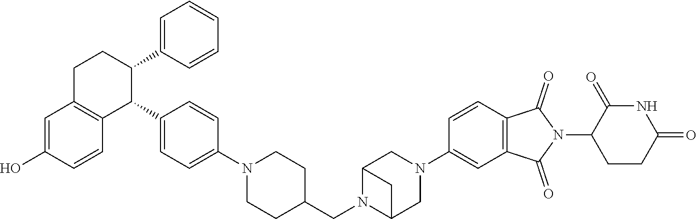 [SMILES: O=C1CCC(N2C(=O)c3ccc(N4CC5CC(C4)N5CC4CCN(c5ccc([C@@H]6c7ccc(O)cc7CC[C@@H]6c6ccccc6)cc5)CC4)cc3C2=O)C(=O)N1] | 2-(2,6-dioxopiperidin-3-yl)-5-(6-((1-(4- ((1R,2S)-6-hydroxy-2-phenyl-1,2,3,4- tetrahydronaphthalen-1-yl)phenyl)piperidin-4- yl)methyl)-3,6-diazabicyclo[3.1.1]heptan-3- yl)isoindoline-1,3-dione |
| | 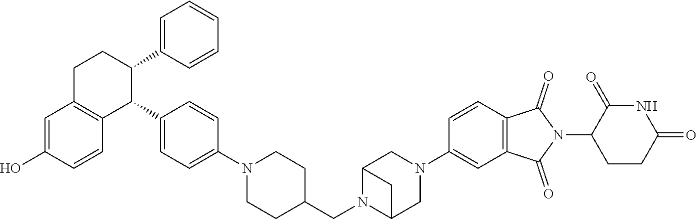 [SMILES: O=C1CCC(N2C(=O)c3ccc(N4CC5CC(C4)N5CC4CCN(c5ccc([C@@H]6c7ccc(O)cc7CC[C@@H]6c6ccccc6)cc5)CC4)cc3C2=O)C(=O)N1] | 2-(2,6-dioxopiperidin-3-yl)-5-fluoro-6-(6-((1- (4-((1R,2S)-6-hydroxy-2-phenyl-1,2,3,4- tetrahydronaphthalen-1-yl)phenyl)piperidin-4- yl)methyl)-3,6-diazabicyclo[3.1.1]heptan-3- yl)isoindoline-1,3-dione |
| | 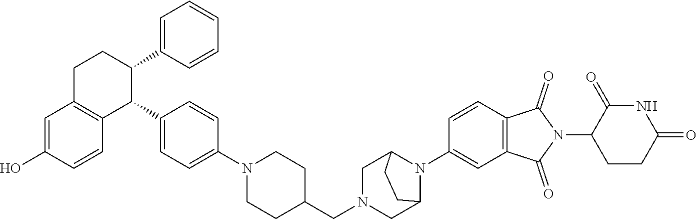 [SMILES: O=C1CCC(N2C(=O)c3ccc(N4C5CCC4CN(CC4CCN(c6ccc([C@@H]7c8ccc(O)cc8CC[C@@H]7c7ccccc7)cc6)CC4)C5)cc3C2=O)C(=O)N1] | 2-(2,6-dioxopiperidin-3-yl)-5-(3-((1-(4- ((1R,2S)-6-hydroxy-2-phenyl-1,2,3,4- tetrahydronaphthalen-1-yl)phenyl)piperidin-4- yl)methyl)-3,8-diazabicyclo[3.2.1]octan-8- yl)isoindoline-1,3-dione |
| | 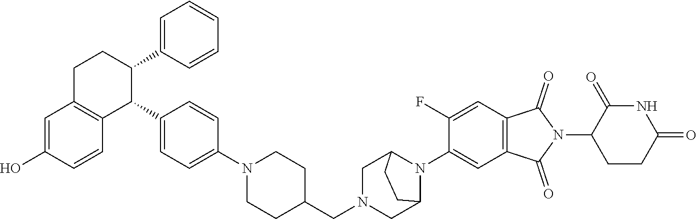 [SMILES: O=C1CCC(N2C(=O)c3cc(F)c(N4C5CCC4CN(CC4CCN(c6ccc([C@@H]7c8ccc(O)cc8CC[C@@H]7c7ccccc7)cc6)CC4)C5)cc3C2=O)C(=O)N1] | 2-(2,6-dioxopiperidin-3-yl)-5-fluoro-6-(3-((1- (4-((1R,2S)-6-hydroxy-2-phenyl-1,2,3,4- tetrahydronaphthalen-1-yl)phenyl)piperidin-4- yl)methyl)-3,8-diazabicyclo[3.2.1]octan-8- yl)isoindoline-1,3-dione |
| | 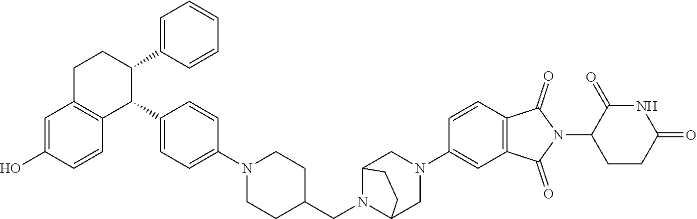 [SMILES: O=C1CCC(N2C(=O)c3ccc(N4CC5CCC(C4)N5CC4CCN(c5ccc([C@@H]6c7ccc(O)cc7CC[C@@H]6c6ccccc6)cc5)CC4)cc3C2=O)C(=O)N1] | 2-(2,6-dioxopiperidin-3-yl)-5-(8-((1-(4- ((1R,2S)-6-hydroxy-2-phenyl-1,2,3,4- tetrahydronaphthalen-1-yl)phenyl)piperidin-4- yl)methyl)-3,8-diazabicyclo[3.2.1]octan-3- yl)isoindoline-1,3-dione |
| | 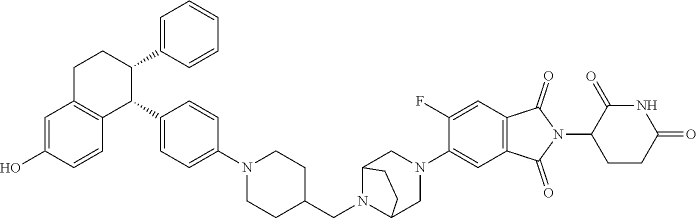 [SMILES: O=C1CCC(N2C(=O)c3cc(F)c(N4CC5CCC(C4)N5CC4CCN(c5ccc([C@@H]6c7ccc(O)cc7CC[C@@H]6c6ccccc6)cc5)CC4)cc3C2=O)C(=O)N1] | 2-(2,6-dioxopiperidin-3-yl)-5-fluoro-6-(8-((1- (4-((1R,2S)-6-hydroxy-2-phenyl-1,2,3,4- tetrahydronaphthalen-1-yl)phenyl)piperidin-4- yl)methyl)-3,8-diazabicyclo[3.2.1]octan-3- yl)isoindoline-1,3-dione |
| | 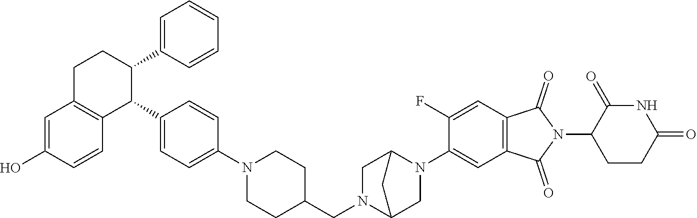 [SMILES: O=C1CCC(N2C(=O)c3cc(F)c(N4CC5CC4CN5CC4CCN(c5ccc([C@@H]6c7ccc(O)cc7CC[C@@H]6c6ccccc6)cc5)CC4)cc3C2=O)C(=O)N1] | 2-(2,6-dioxopiperidin-3-yl)-5-fluoro-6-(5-((1- (4-((1R,2S)-6-hydroxy-2-phenyl-1,2,3,4- tetrahydronaphthalen-1-yl)phenyl)piperidin-4- yl)methyl)-2,5-diazabicyclo[2.2.1]heptan-2- yl)isoindoline-1,3-dione |
| | 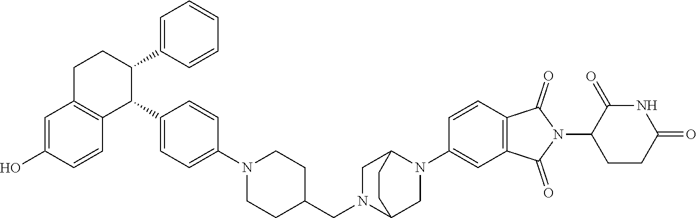 [SMILES: O=C1CCC(N2C(=O)c3ccc(N4CC5CCC4CN5CC4CCN(c5ccc([C@@H]6c7ccc(O)cc7CC[C@@H]6c6ccccc6)cc5)CC4)cc3C2=O)C(=O)N1] | 2-(2,6-dioxopiperidin-3-yl)-5-(5-((1-(4- ((1R,2S)-6-hydroxy-2-phenyl-1,2,3,4- tetrahydronaphthalen-1-yl)phenyl)piperidin-4- yl)methyl)-2,5-diazabicyclo[2.2.2]octan-2- yl)isoindoline-1,3-dione |
| | 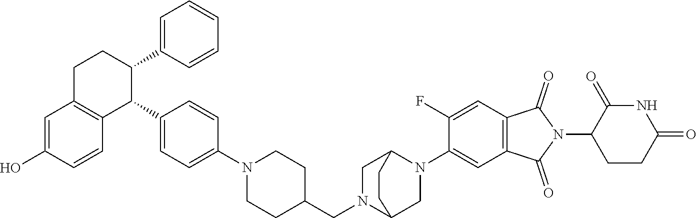 [SMILES: O=C1CCC(N2C(=O)c3cc(F)c(N4CC5CCC4CN5CC4CCN(c5ccc([C@@H]6c7ccc(O)cc7CC[C@@H]6c6ccccc6)cc5)CC4)cc3C2=O)C(=O)N1] | 2-(2,6-dioxopiperidin-3-yl)-5-fluoro-6-(5-((1- (4-((1R,2S)-6-hydroxy-2-phenyl-1,2,3,4- tetrahydronaphthalen-1-yl)phenyl)piperidin-4- yl)methyl)-2,5-diazabicyclo[2.2.2]octan-2- yl)isoindoline-1,3-dione |
| | 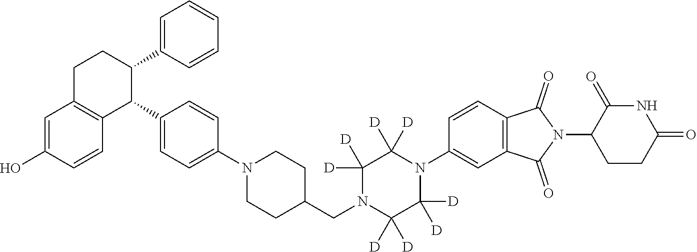 [SMILES: [2H]C1([2H])N(CC2CCN(c3ccc([C@@H]4c5ccc(O)cc5CC[C@@H]4c4ccccc4)cc3)CC2)C([2H])([2H])C([2H])([2H])N(c2ccc3c(c2)C(=O)N(C2CCC(=O)NC2=O)C3=O)C1([2H])[2H]] | 2-(2,6-dioxopiperidin-3-yl)-5-(4-((1-(4- ((1R,2S)-6-hydroxy-2-phenyl-1,2,3,4- tetrahydronaphthalen-1-yl)phenyl)piperidin-4- yl)methyl)piperazin-1-yl-2,2,3,3,5,5,6,6- d₈)isoindoline-1,3-dione |
| | 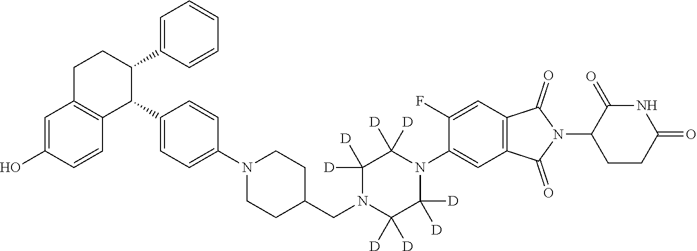 [SMILES: [2H]C1([2H])N(CC2CCN(c3ccc([C@@H]4c5ccc(O)cc5CC[C@@H]4c4ccccc4)cc3)CC2)C([2H])([2H])C([2H])([2H])N(c2cc3c(cc2F)C(=O)N(C2CCC(=O)NC2=O)C3=O)C1([2H])[2H]] | 2-(2,6-dioxopiperidin-3-yl)-5-fluoro-6-(4-((1- (4-((1R,2S)-6-hydroxy-2-phenyl-1,2,3,4- tetrahydronaphthalen-1-yl)phenyl)piperidin-4- yl)methyl)piperazin-1-yl-2,2,3,3,5,5,6,6- d₈)isoindoline-1,3-dione |
| | 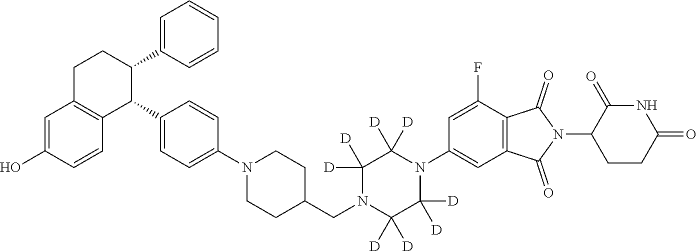 [SMILES: [2H]C1([2H])N(CC2CCN(c3ccc([C@@H]4c5ccc(O)cc5CC[C@@H]4c4ccccc4)cc3)CC2)C([2H])([2H])C([2H])([2H])N(c2cc(F)c3c(c2)C(=O)N(C2CCC(=O)NC2=O)C3=O)C1([2H])[2H]] | 2-(2,6-dioxopiperidin-3-yl)-4-fluoro-6-(4-((1- (4-((1R,2S)-6-hydroxy-2-phenyl-1,2,3,4- tetrahydronaphthalen-1-yl)phenyl)piperidin-4- yl)methyl)piperazin-1-yl-2,2,3,3,5,5,6,6- d₈)isoindoline-1,3-dione |
| | 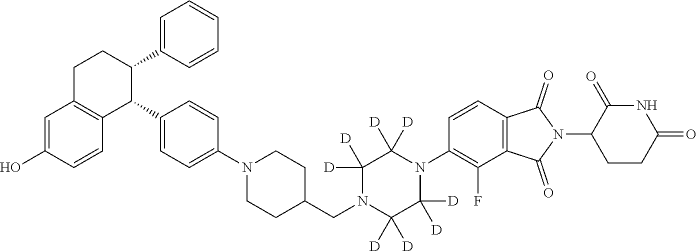 [SMILES: [2H]C1([2H])N(CC2CCN(c3ccc([C@@H]4c5ccc(O)cc5CC[C@@H]4c4ccccc4)cc3)CC2)C([2H])([2H])C([2H])([2H])N(c2ccc3c(c2F)C(=O)N(C2CCC(=O)NC2=O)C3=O)C1([2H])[2H]] | 2-(2,6-dioxopiperidin-3-yl)-4-fluoro-5-(4-((1- (4-((1R,2S)-6-hydroxy-2-phenyl-1,2,3,4- tetrahydronaphthalen-1-yl)phenyl)piperidin-4- yl)methyl)piperazin-1-yl-2,2,3,3,5,5,6,6- d₈)isoindoline-1,3-dione |
| | 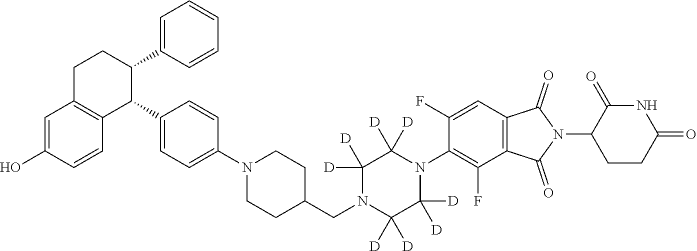 [SMILES: [2H]C1([2H])N(CC2CCN(c3ccc([C@@H]4c5ccc(O)cc5CC[C@@H]4c4ccccc4)cc3)CC2)C([2H])([2H])C([2H])([2H])N(c2c(F)cc3c(c2F)C(=O)N(C2CCC(=O)NC2=O)C3=O)C1([2H])[2H]] | 2-(2,6-dioxopiperidin-3-yl)-4,6-difluoro-5-(4- ((1-(4-((1R,2S)-6-hydroxy-2-phenyl-1,2,3,4- tetrahydronaphthalen-1-yl)phenyl)piperidin-4- yl)methyl)piperazin-1-yl-2,2,3,3,5,5,6,6- d₈)isoindoline-1,3-dione |
| | 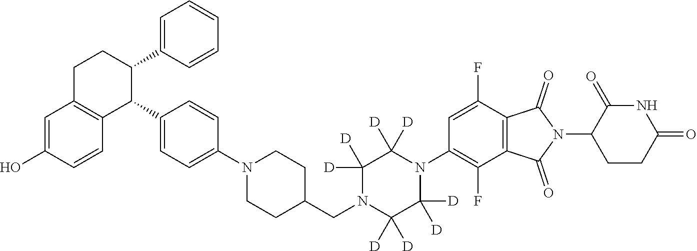 [SMILES: [2H]C1([2H])N(CC2CCN(c3ccc([C@@H]4c5ccc(O)cc5CC[C@@H]4c4ccccc4)cc3)CC2)C([2H])([2H])C([2H])([2H])N(c2cc(F)c3c(c2F)C(=O)N(C2CCC(=O)NC2=O)C3=O)C1([2H])[2H]] | 2-(2,6-dioxopiperidin-3-yl)-4,7-difluoro-5-(4- ((1-(4-((1R,2S)-6-hydroxy-2-phenyl-1,2,3,4- tetrahydronaphthalen-1-yl)phenyl)piperidin-4- yl)methyl)piperazin-1-yl-2,2,3,3,5,5,6,6- d₈)isoindoline-1,3-dione |
| | 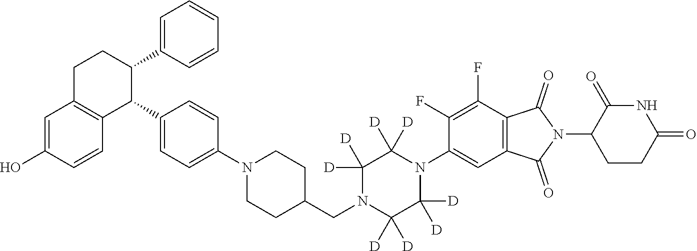 [SMILES: [2H]C1([2H])N(CC2CCN(c3ccc([C@@H]4c5ccc(O)cc5CC[C@@H]4c4ccccc4)cc3)CC2)C([2H])([2H])C([2H])([2H])N(c2cc3c(c(F)c2F)C(=O)N(C2CCC(=O)NC2=O)C3=O)C1([2H])[2H]] | 2-(2,6-dioxopiperidin-3-yl)-4,5-difluoro-6-(4- ((1-(4-((1R,2S)-6-hydroxy-2-phenyl-1,2,3,4- tetrahydronaphthalen-1-yl)phenyl)piperidin-4- yl)methyl)piperazin-1-yl-2,2,3,3,5,5,6,6- d₈)isoindoline-1,3-dione |
| | 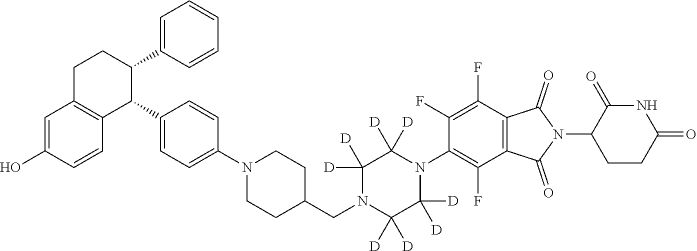 [SMILES: [2H]C1([2H])N(CC2CCN(c3ccc([C@@H]4c5ccc(O)cc5CC[C@@H]4c4ccccc4)cc3)CC2)C([2H])([2H])C([2H])([2H])N(c2c(F)c(F)c3c(c2F)C(=O)N(C2CCC(=O)NC2=O)C3=O)C1([2H])[2H]] | 2-(2,6-dioxopiperidin-3-yl)-4,5,7-trifluoro-6- (4-((1-(4-((1R,2S)-6-hydroxy-2-phenyl-1,2,3,4- tetrahydronaphthalen-1-yl)phenyl)piperidin-4- yl)methyl)piperazin-1-yl-2,2,3,3,5,5,6,6- d₈)isoindoline-1,3-dione |
| | 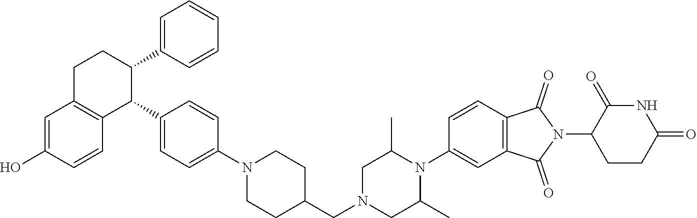 [SMILES: CC1CN(CC2CCN(c3ccc([C@@H]4c5ccc(O)cc5CC[C@@H]4c4ccccc4)cc3)CC2)CC(C)N1c1ccc2c(c1)C(=O)N(C1CCC(=O)NC1=O)C2=O] | 2-(2,6-dioxopiperidin-3-yl)-5-(4-((1-(4- ((1R,2S)-6-hydroxy-2-phenyl-1,2,3,4- tetrahydronaphthalen-1-yl)phenyl)piperidin-4- yl)methyl)-2,6-dimethylpiperazin-1- yl)isoindoline-1,3-dione |
| | 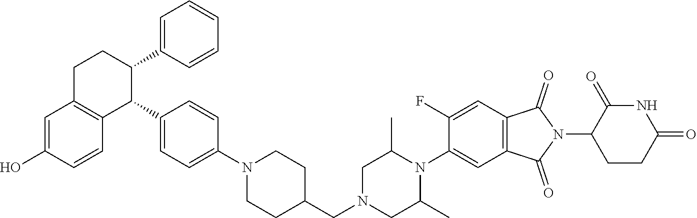 [SMILES: CC1CN(CC2CCN(c3ccc([C@@H]4c5ccc(O)cc5CC[C@@H]4c4ccccc4)cc3)CC2)CC(C)N1c1cc2c(cc1F)C(=O)N(C1CCC(=O)NC1=O)C2=O] | 2-(2,6-dioxopiperidin-3-yl)-5-fluoro-6-(4-((1- (4-((1R,2S)-6-hydroxy-2-phenyl-1,2,3,4- tetrahydronaphthalen-1-yl)phenyl)piperidin-4- yl)methyl)-2,6-dimethylpiperazin-1- yl)isoindoline-1,3-dione |
| | 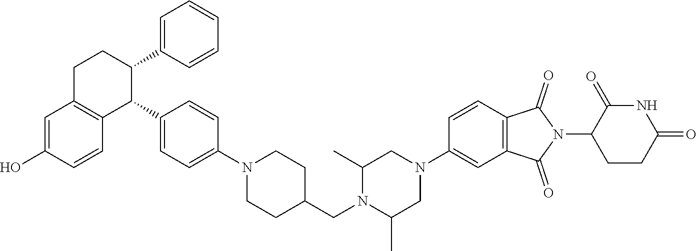 [SMILES: CC1CN(c2ccc3c(c2)C(=O)N(C2CCC(=O)NC2=O)C3=O)CC(C)N1CC1CCN(c2ccc([C@@H]3c4ccc(O)cc4CC[C@@H]3c3ccccc3)cc2)CC1] | 2-(2,6-dioxopiperidin-3-yl)-5-(4-((1-(4- ((1R,2S)-6-hydroxy-2-phenyl-1,2,3,4- tetrahydronaphthalen-1-yl)phenyl)piperidin-4- yl)methyl)-3,5-dimethylpiperazin-1- yl)isoindoline-1,3-dione |
| | 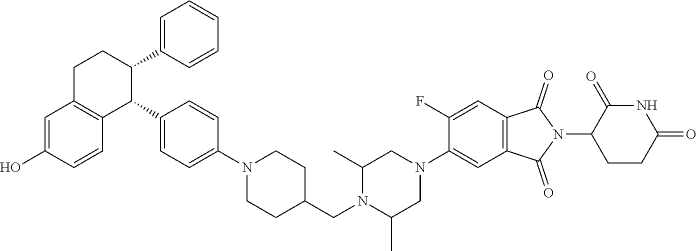 [SMILES: CC1CN(c2cc3c(cc2F)C(=O)N(C2CCC(=O)NC2=O)C3=O)CC(C)N1CC1CCN(c2ccc([C@@H]3c4ccc(O)cc4CC[C@@H]3c3ccccc3)cc2)CC1] | 2-(2,6-dioxopiperidin-3-yl)-5-fluoro-6-(4-((1- (4-((1R,2S)-6-hydroxy-2-phenyl-1,2,3,4- tetrahydronaphthalen-1-yl)phenyl)piperidin-4- yl)methyl)-3,5-dimethylpiperazin-1- yl)isoindoline-1,3-dione |
| | 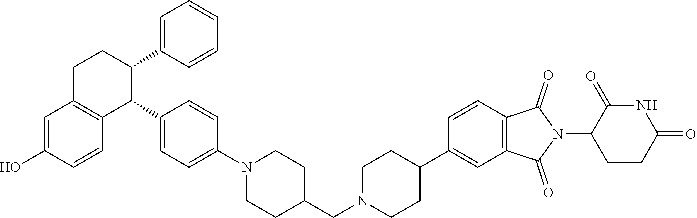 [SMILES: O=C1CCC(N2C(=O)c3ccc(C4CCN(CC5CCN(c6ccc([C@@H]7c8ccc(O)cc8CC[C@@H]7c7ccccc7)cc6)CC5)CC4)cc3C2=O)C(=O)N1] | 2-(2,6-dioxopiperidin-3-yl)-5-(1-((1-(4- ((1R,2S)-6-hydroxy-2-phenyl-1,2,3,4- tetrahydronaphthalen-1-yl)phenyl)piperidin-4- yl)methyl)piperidin-4-yl)isoindoline-1,3-dione |
| | 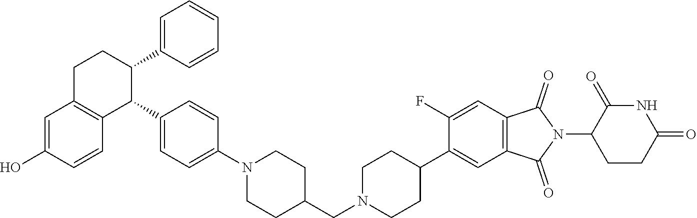 [SMILES: O=C1CCC(N2C(=O)c3cc(F)c(C4CCN(CC5CCN(c6ccc([C@@H]7c8ccc(O)cc8CC[C@@H]7c7ccccc7)cc6)CC5)CC4)cc3C2=O)C(=O)N1] | 2-(2,6-dioxopiperidin-3-yl)-5-fluoro-6-(1-((1- (4-((1R,2S)-6-hydroxy-2-phenyl-1,2,3,4- tetrahydronaphthalen-1-yl)phenyl)piperidin-4- yl)methyl)piperidin-4-yl)isoindoline-1,3-dione |
| | 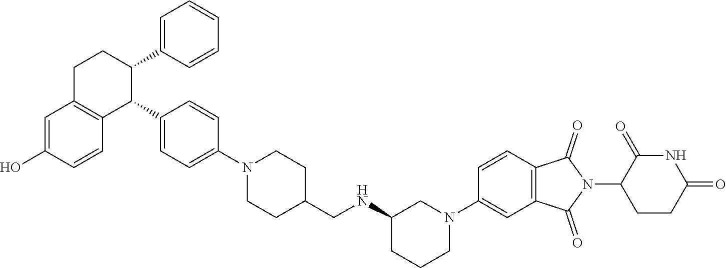 [SMILES: O=C1CCC(N2C(=O)c3ccc(N4CCC[C@@H](NCC5CCN(c6ccc([C@@H]7c8ccc(O)cc8CC[C@@H]7c7ccccc7)cc6)CC5)C4)cc3C2=O)C(=O)N1] | 2-(2,6-dioxopiperidin-3-yl)-5-((R)-3-(((1-(4- ((1R,2S)-6-hydroxy-2-phenyl-1,2,3,4- tetrahydronaphthalen-1-yl)phenyl)piperidin-4- yl)methyl)amino)piperidin-1-yl)isoindoline- 1,3-dione |
| | 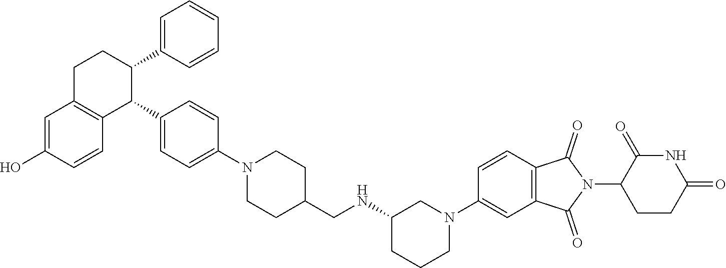 [SMILES: O=C1CCC(N2C(=O)c3ccc(N4CCC[C@H](NCC5CCN(c6ccc([C@@H]7c8ccc(O)cc8CC[C@@H]7c7ccccc7)cc6)CC5)C4)cc3C2=O)C(=O)N1] | 2-(2,6-dioxopiperidin-3-yl)-5-((S)-3-(((1-(4- ((1R,2S)-6-hydroxy-2-phenyl-1,2,3,4- tetrahydronaphthalen-1-yl)phenyl)piperidin-4- yl)methyl)amino)piperidin-1-yl)isoindoline- 1,3-dione |
| | 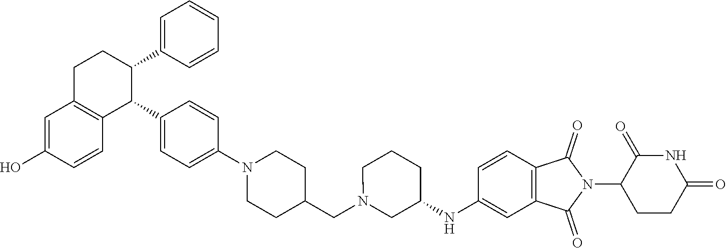 [SMILES: O=C1CCC(N2C(=O)c3ccc(N[C@H]4CCCN(CC5CCN(c6ccc([C@@H]7c8ccc(O)cc8CC[C@@H]7c7ccccc7)cc6)CC5)C4)cc3C2=O)C(=O)N1] | 2-(2,6-dioxopiperidin-3-yl)-5-(((S)-1-((1-(4- ((1R,2S)-6-hydroxy-2-phenyl-1,2,3,4- tetrahydronaphthalen-1-yl)phenyl)piperidin-4- yl)methyl)piperidin-3-yl)amino)isoindoline- 1,3-dione |
| | 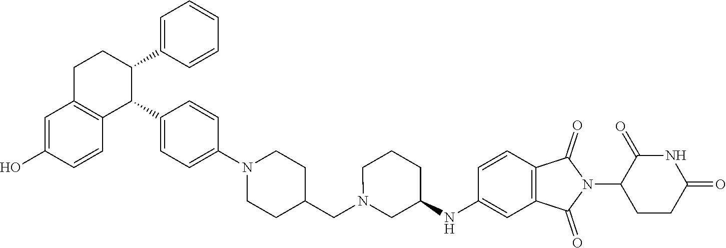 [SMILES: O=C1CCC(N2C(=O)c3ccc(N[C@@H]4CCCN(CC5CCN(c6ccc([C@@H]7c8ccc(O)cc8CC[C@@H]7c7ccccc7)cc6)CC5)C4)cc3C2=O)C(=O)N1] | 2-(2,6-dioxopiperidin-3-yl)-5-(((R)-1-((1-(4- ((1R,2S)-6-hydroxy-2-phenyl-1,2,3,4- tetrahydronaphthalen-1-yl)phenyl)piperidin-4- yl)methyl)piperidin-3-yl)amino)isoindoline- 1,3-dione |
| | 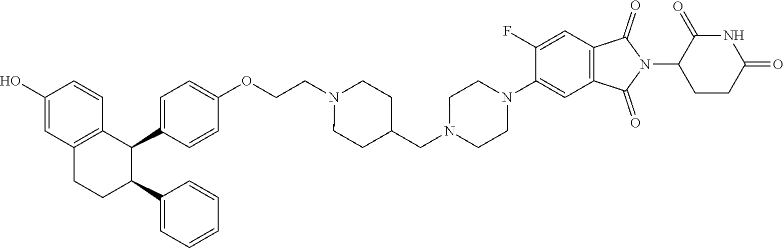 [SMILES: O=C1CCC(N2C(=O)c3cc(F)c(N4CCN(CC5CCN(CCOc6ccc([C@@H]7c8ccc(O)cc8CC[C@@H]7c7ccccc7)cc6)CC5)CC4)cc3C2=O)C(=O)N1] | 2-(2,6-dioxopiperidin-3-yl)-5-fluoro-6-(4-((1- (2-(4-((1R,2S)-6-hydroxy-2-phenyl-1,2,3,4- tetrahydronaphthalen-1 - yl)phenoxy)ethyl)piperidin-4- yl)methyl)piperazin-1-yl)isoindoline-1,3-dione |
| | 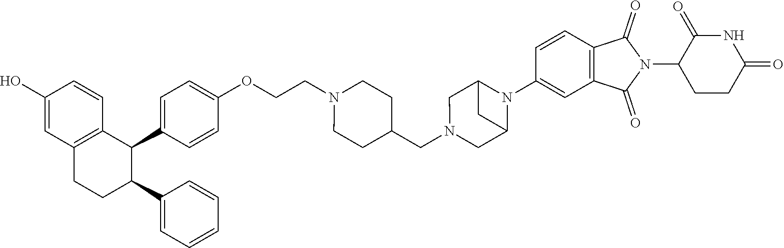 [SMILES: O=C1CCC(N2C(=O)c3ccc(N4C5CC4CN(CC4CCN(CCOc6ccc([C@@H]7c8ccc(O)cc8CC[C@@H]7c7ccccc7)cc6)CC4)C5)cc3C2=O)C(=O)N1] | 2-(2,6-dioxopiperidin-3-yl)-5-(3-((1-(2-(4- ((1R,2S)-6-hydroxy-2-phenyl-1,2,3,4- tetrahydronaphthalen-1- yl)phenoxy)ethyl)piperidin-4-yl)methyl)-3,6- diazabicyclo[3.1.1]heptan-6-yl)isoindoline-1,3- dione |
| | 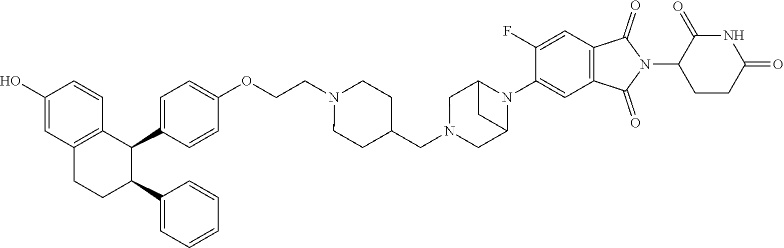 [SMILES: O=C1CCC(N2C(=O)c3cc(F)c(N4C5CC4CN(CC4CCN(CCOc6ccc([C@@H]7c8ccc(O)cc8CC[C@@H]7c7ccccc7)cc6)CC4)C5)cc3C2=O)C(=O)N1] | 2-(2,6-dioxopiperidin-3-yl)-5-fluoro-6-(3-((1- (2-(4-((1R,2S)-6-hydroxy-2-phenyl-1,2,3,4- tetrahydronaphthalen-1- yl)phenoxy)ethyl)piperidin-4-yl)methyl)-3,6- diazabicyclo[3.1.1]heptan-6-yl)isoindoline-1,3- dione |
| | 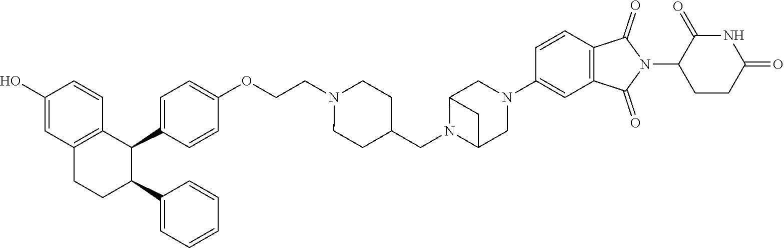 [SMILES: O=C1CCC(N2C(=O)c3ccc(N4CC5CC(C4)N5CC4CCN(CCOc5ccc([C@@H]6c7ccc(O)cc7CC[C@@H]6c6ccccc6)cc5)CC4)cc3C2=O)C(=O)N1] | 2-(2,6-dioxopiperidin-3-yl)-5-(6-((1-(2-(4- ((1R,2S)-6-hydroxy-2-phenyl-1,2,3,4- tetrahydronaphthalen-1- yl)phenoxy)ethyl)piperidin-4-yl)methyl)-3,6- diazabicyclo[3.1.1]heptan-3-yl)isoindoline-1,3- dione |
| | 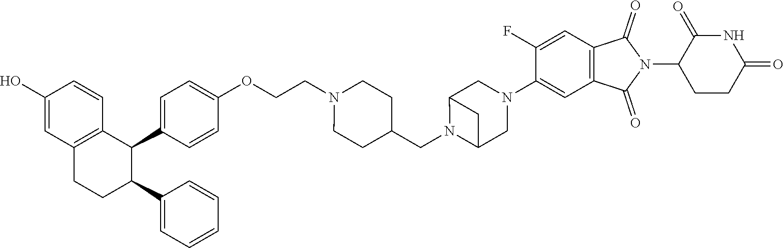 [SMILES: O=C1CCC(N2C(=O)c3cc(F)c(N4CC5CC(C4)N5CC4CCN(CCOc5ccc([C@@H]6c7ccc(O)cc7CC[C@@H]6c6ccccc6)cc5)CC4)cc3C2=O)C(=O)N1] | 2-(2,6-dioxopiperidin-3-yl)-5-fluoro-6-(6-((1- (2-(4-((1R,2S)-6-hydroxy-2-phenyl-1,2,3,4- tetrahydronaphthalen-1- yl)phenoxy)ethyl)piperidin-4-yl)methyl)-3,6- diazabicyclo[3.1.1]heptan-3-yl)isoindoline-1,3- dione |
| | 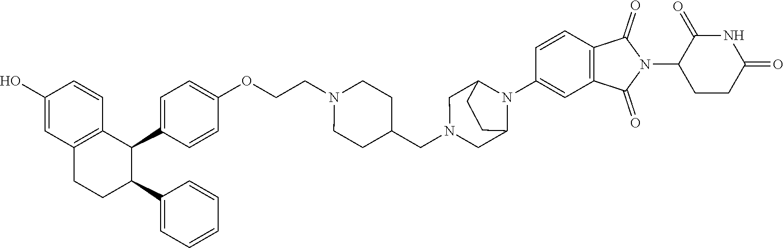 [SMILES: O=C1CCC(N2C(=O)c3ccc(N4C5CCC4CN(CC4CCN(CCOc6ccc([C@@H]7c8ccc(O)cc8CC[C@@H]7c7ccccc7)cc6)CC4)C5)cc3C2=O)C(=O)N1] | 2-(2,6-dioxopiperidin-3-yl)-5-(3-((1-(2-(4- ((1R,2S)-6-hydroxy-2-phenyl-1,2,3,4- tetrahydronaphthalen-1- yl)phenoxy)ethyl)piperidin-4-yl)methyl)-3,8- diazabicyclo[3.2.1]octan-8-yl)isoindoline-1,3- dione |
| | 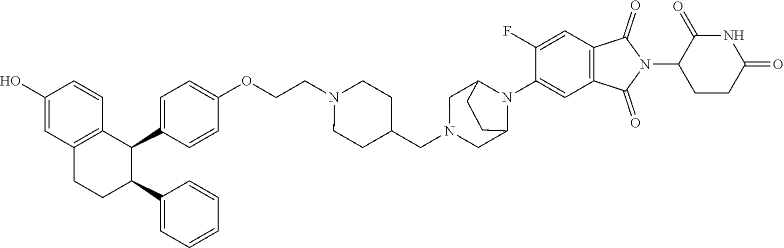 [SMILES: O=C1CCC(N2C(=O)c3cc(F)c(N4C5CCC4CN(CC4CCN(CCOc6ccc([C@@H]7c8ccc(O)cc8CC[C@@H]7c7ccccc7)cc6)CC4)C5)cc3C2=O)C(=O)N1] | 2-(2,6-dioxopiperidin-3-yl)-5-fluoro-6-(3-((1- (2-(4-((1R,2S)-6-hydroxy-2-phenyl-1,2,3,4- tetrahydronaphthalen-1- yl)phenoxy)ethyl)piperidin-4-yl)methyl)-3,8- diazabicyclo[3.2.1]octan-8-yl)isoindoline-1,3- dione |
| | 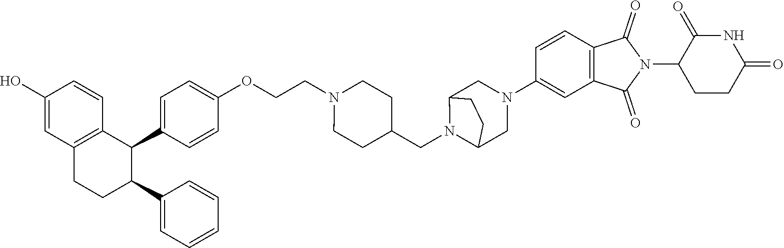 [SMILES: O=C1CCC(N2C(=O)c3ccc(N4CC5CCC(C4)N5CC4CCN(CCOc5ccc([C@@H]6c7ccc(O)cc7CC[C@@H]6c6ccccc6)cc5)CC4)cc3C2=O)C(=O)N1] | 2-(2,6-dioxopiperidin-3-yl)-5-(8-((1-(2-(4- ((1R,2S)-6-hydroxy-2-phenyl-1,2,3,4- tetrahydronaphthalen-1- yl)phenoxy)ethyl)piperidin-4-yl)methyl)-3,8- diazabicyclo[3.2.1]octan-3-yl)isoindoline-1,3- dione |
| | 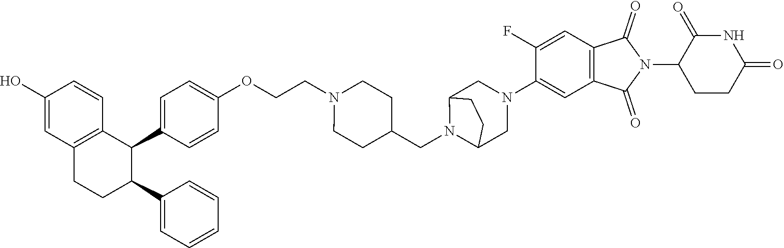 [SMILES: O=C1CCC(N2C(=O)c3cc(F)c(N4CC5CCC(C4)N5CC4CCN(CCOc5ccc([C@@H]6c7ccc(O)cc7CC[C@@H]6c6ccccc6)cc5)CC4)cc3C2=O)C(=O)N1] | 2-(2,6-dioxopiperidin-3-yl)-5-fluoro-6-(8-((1- (2-(4-((1R,2S)-6-hydroxy-2-phenyl-1,2,3,4- tetrahydronaphthalen-1- yl)phenoxy)ethyl)piperidin-4-yl)methyl)-3,8- diazabicyclo[3.2.1]octan-3-yl)isoindoline-1,3- dione |
| | 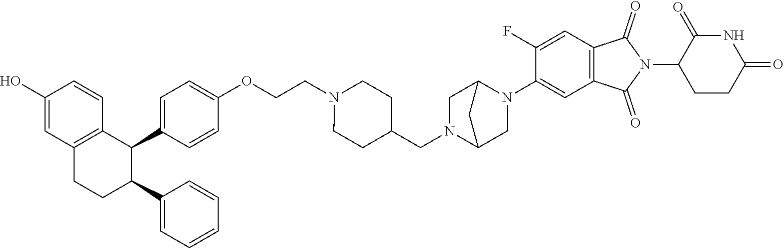 [SMILES: O=C1CCC(N2C(=O)c3cc(F)c(N4CC5CC4CN5CC4CCN(CCOc5ccc([C@@H]6c7ccc(O)cc7CC[C@@H]6c6ccccc6)cc5)CC4)cc3C2=O)C(=O)N1] | 2-(2,6-dioxopiperidin-3-yl)-5-fluoro-6-(5-((1- (2-(4-((1R,2S)-6-hydroxy-2-phenyl-1,2,3,4- tetrahydronaphthalen-1- yl)phenoxy)ethyl)piperidin-4-yl)methyl)-2,5- diazabicyclo[2.2.1]heptan-2-yl)isoindoline-1,3- dione |
| | 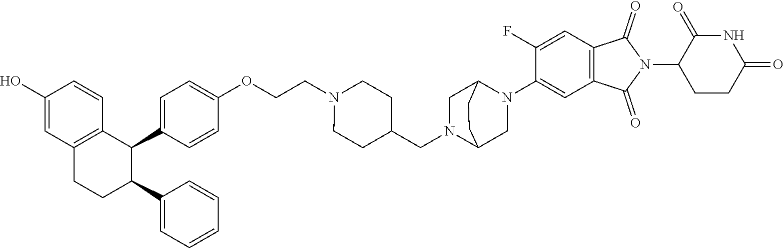 [SMILES: O=C1CCC(N2C(=O)c3cc(F)c(N4CC5CCC4CN5CC4CCN(CCOc5ccc([C@@H]6c7ccc(O)cc7CC[C@@H]6c6ccccc6)cc5)CC4)cc3C2=O)C(=O)N1] | 2-(2,6-dioxopiperidin-3-yl)-5-fluoro-6-(5-((1- (2-(4-((1R,2S)-6-hydroxy-2-phenyl-1,2,3,4- tetrahydronaphthalen-1- yl)phenoxy)ethyl)piperidin-4-yl)methyl)-2,5- diazabicyclo[2.2.2]octan-2-yl)isoindoline-1,3- dione |
| | 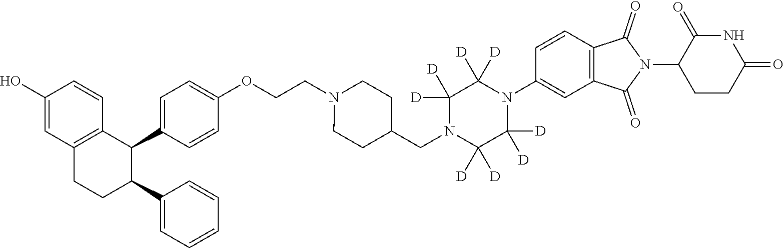 [SMILES: [2H]C1([2H])N(CC2CCN(CCOc3ccc([C@@H]4c5ccc(O)cc5CC[C@@H]4c4ccccc4)cc3)CC2)C([2H])([2H])C([2H])([2H])N(c2ccc3c(c2)C(=O)N(C2CCC(=O)NC2=O)C3=O)C1([2H])[2H]] | 2-(2,6-dioxopiperidin-3-yl)-5-(4-((1-(2-(4- ((1R,2S)-6-hydroxy-2-phenyl-1,2,3,4- tetrahydronaphthalen-1- yl)phenoxy)ethyl)piperidin-4- yl)methyl)piperazin-1-yl-2,2,3,3,5,5,6,6- d₈)isoindoline-1,3-dione |
| | 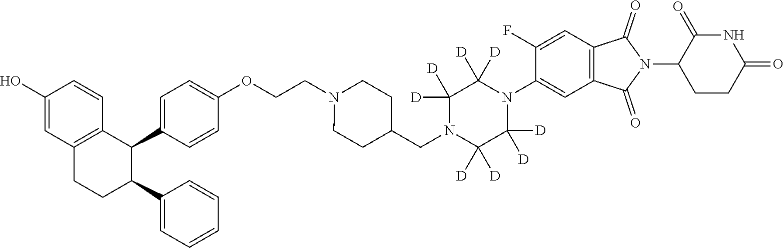 [SMILES: [2H]C1([2H])N(CC2CCN(CCOc3ccc([C@@H]4c5ccc(O)cc5CC[C@@H]4c4ccccc4)cc3)CC2)C([2H])([2H])C([2H])([2H])N(c2cc3c(cc2F)C(=O)N(C2CCC(=O)NC2=O)C3=O)C1([2H])[2H]] | 2-(2,6-dioxopiperidin-3-yl)-5-fluoro-6-(4-((1- (2-(4-((1R,2S)-6-hydroxy-2-phenyl-1,2,3,4- tetrahydronaphthalen-1- yl)phenoxy)ethyl)piperidin-4- yl)methyl)piperazin-1-yl-2,2,3,3,5,5,6,6- d₈)isoindoline-1,3-dione |
| | 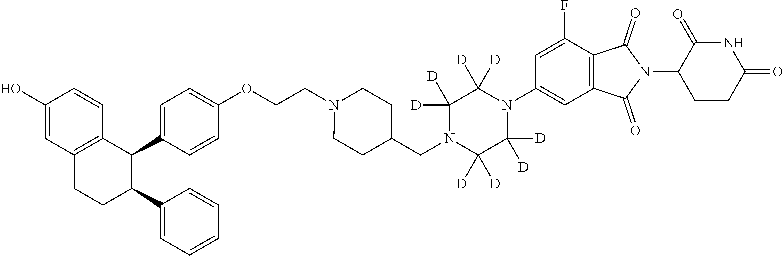 [SMILES: [2H]C1([2H])N(CC2CCN(CCOc3ccc([C@@H]4c5ccc(O)cc5CC[C@@H]4c4ccccc4)cc3)CC2)C([2H])([2H])C([2H])([2H])N(c2cc(F)c3c(c2)C(=O)N(C2CCC(=O)NC2=O)C3=O)C1([2H])[2H]] | 2-(2,6-dioxopiperidin-3-yl)-4-fluoro-6-(4-((1- (2-(4-((1R,2S)-6-hydroxy-2-phenyl-1,2,3,4- tetrahydronaphthalen-1- yl)phenoxy)ethyl)piperidin-4- yl)methyl)piperazin-1-yl-2,2,3,3,5,5,6,6- d₈)isoindoline-1,3-dione |
| | 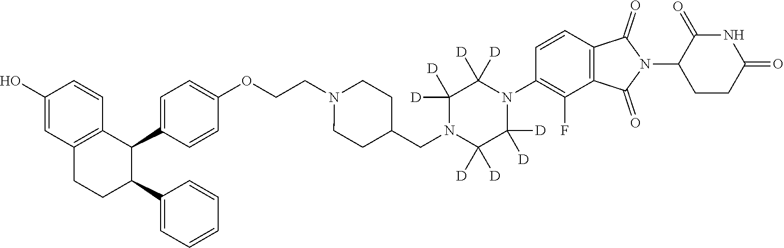 [SMILES: [2H]C1([2H])N(CC2CCN(CCOc3ccc([C@@H]4c5ccc(O)cc5CC[C@@H]4c4ccccc4)cc3)CC2)C([2H])([2H])C([2H])([2H])N(c2ccc3c(c2F)C(=O)N(C2CCC(=O)NC2=O)C3=O)C1([2H])[2H]] | 2-(2,6-dioxopiperidin-3-yl)-4-fluoro-5-(4-((1- (2-(4-((1R,2S)-6-hydroxy-2-phenyl-1,2,3,4- tetrahydronaphthalen-1- yl)phenoxy)ethyl)piperidin-4- yl)methyl)piperazin-1-yl-2,2,3,3,5,5,6,6- d₈)isoindoline-1,3-dione |
| | 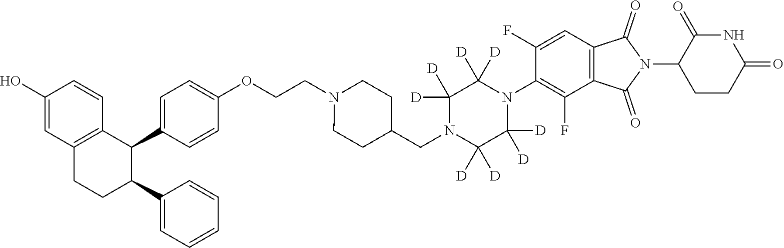 [SMILES: [2H]C1([2H])N(CC2CCN(CCOc3ccc([C@@H]4c5ccc(O)cc5CC[C@@H]4c4ccccc4)cc3)CC2)C([2H])([2H])C([2H])([2H])N(c2c(F)cc3c(c2F)C(=O)N(C2CCC(=O)NC2=O)C3=O)C1([2H])[2H]] | 2-(2,6-dioxopiperidin-3-yl)-4,6-difluoro-5-(4- ((1-(2-(4-((1R,2S)-6-hydroxy-2-phenyl-1,2,3,4- tetrahydronaphthalen-1- yl)phenoxy)ethyl)piperidin-4- yl)methyl)piperazin-1-yl-2,2,3,3,5,5,6,6- d₈)isoindoline-1,3-dione |
| | 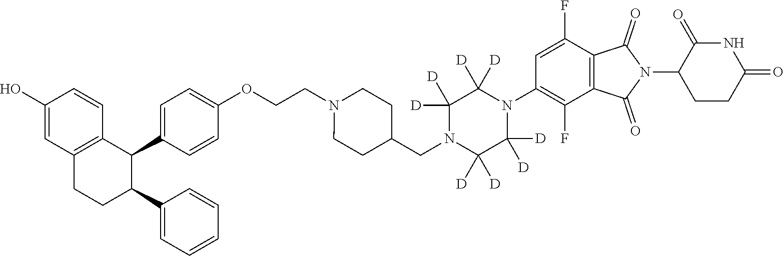 [SMILES: [2H]C1([2H])N(CC2CCN(CCOc3ccc([C@@H]4c5ccc(O)cc5CC[C@@H]4c4ccccc4)cc3)CC2)C([2H])([2H])C([2H])([2H])N(c2cc(F)c3c(c2F)C(=O)N(C2CCC(=O)NC2=O)C3=O)C1([2H])[2H]] | 2-(2,6-dioxopiperidin-3-yl)-4,7-difluoro-5-(4- ((1-(2-(4-((1R,2S)-6-hydroxy-2-phenyl-1,2,3,4- tetrahydronaphthalen-1- yl)phenoxy)ethyl)piperidin-4- yl)methyl)piperazin-1-yl-2,2,3,3,5,5,6,6- d₈)isoindoline-1,3-dione |
| | 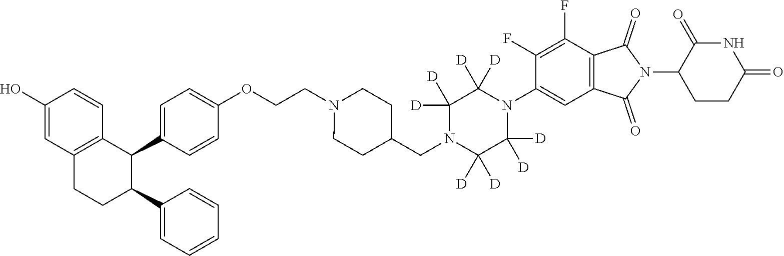 [SMILES: [2H]C1([2H])N(CC2CCN(CCOc3ccc([C@@H]4c5ccc(O)cc5CC[C@@H]4c4ccccc4)cc3)CC2)C([2H])([2H])C([2H])([2H])N(c2cc3c(c(F)c2F)C(=O)N(C2CCC(=O)NC2=O)C3=O)C1([2H])[2H]] | 2-(2,6-dioxopiperidin-3-yl)-4,5-difluoro-6-(4- ((1-(2-(4-((1R,2S)-6-hydroxy-2-phenyl-1,2,3,4- tetrahydronaphthalen-1- yl)phenoxy)ethyl)piperidin-4- yl)methyl)piperazin-1-yl-2,2,3,3,5,5,6,6- d₈)isoindoline-1,3-dione |
| | 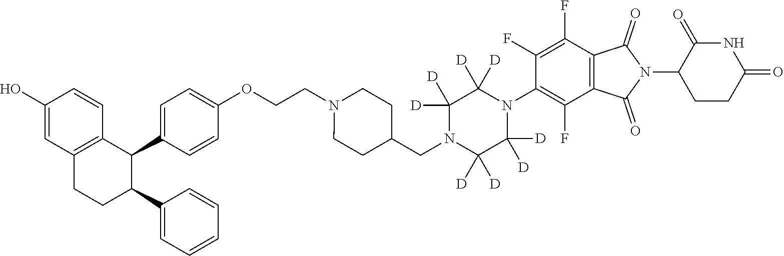 [SMILES: [2H]C1([2H])N(CC2CCN(CCOc3ccc([C@@H]4c5ccc(O)cc5CC[C@@H]4c4ccccc4)cc3)CC2)C([2H])([2H])C([2H])([2H])N(c2c(F)c(F)c3c(c2F)C(=O)N(C2CCC(=O)NC2=O)C3=O)C1([2H])[2H]] | 2-(2,6-dioxopiperidin-3-yl)-4,5,7-trifluoro-6- (4-((1-(2-(4-((1R,2S)-6-hydroxy-2-phenyl- 1,2,3,4-tetrahydronaphthalen-1- yl)phenoxy)ethyl)piperidin-4- yl)methyl)piperazin-1-yl-2,2,3,3,5,5,6,6- d₈)isoindoline-1,3-dione |
| | 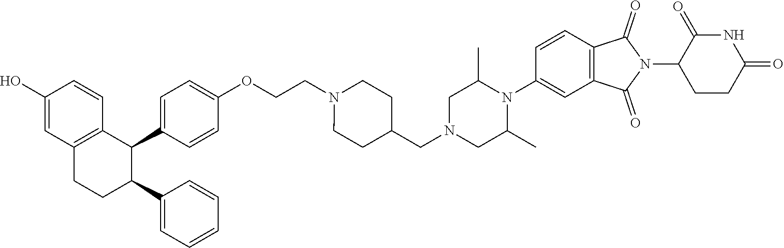 [SMILES: CC1CN(CC2CCN(CCOc3ccc([C@@H]4c5ccc(O)cc5CC[C@@H]4c4ccccc4)cc3)CC2)CC(C)N1c1ccc2c(c1)C(=O)N(C1CCC(=O)NC1=O)C2=O] | 2-(2,6-dioxopiperidin-3-yl)-5-(4-((1-(2-(4- ((1R,2S)-6-hydroxy-2-phenyl-1,2,3,4- tetrahydronaphthalen-1- yl)phenoxy)ethyl)piperidin-4-yl)methyl)-2,6- dimethylpiperazin-1-yl)isoindoline-1,3-dione |
| | 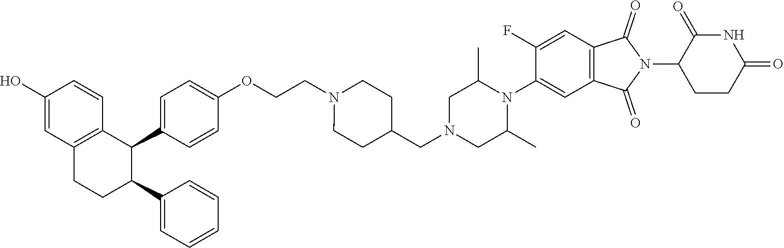 [SMILES: CC1CN(CC2CCN(CCOc3ccc([C@@H]4c5ccc(O)cc5CC[C@@H]4c4ccccc4)cc3)CC2)CC(C)N1c1cc2c(cc1F)C(=O)N(C1CCC(=O)NC1=O)C2=O] | 2-(2,6-dioxopiperidin-3-yl)-5-fluoro-6-(4-((1- (2-(4-((1R,2S)-6-hydroxy-2-phenyl-1,2,3,4- tetrahydronaphthalen-1- yl)phenoxy)ethyl)piperidin-4-yl)methyl)-2,6- dimethylpiperazin-1-yl)isoindoline-1,3-dione |
| | 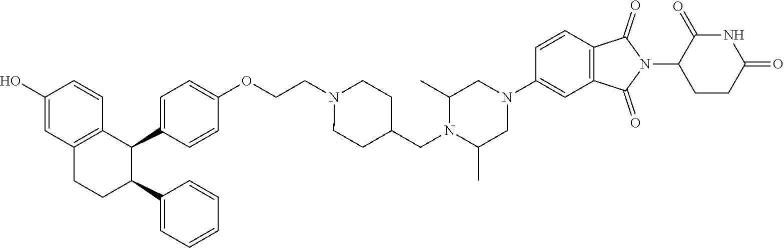 [SMILES: CC1CN(c2ccc3c(c2)C(=O)N(C2CCC(=O)NC2=O)C3=O)CC(C)N1CC1CCN(CCOc2ccc([C@@H]3c4ccc(O)cc4CC[C@@H]3c3ccccc3)cc2)CC1] | 2-(2,6-dioxopiperidin-3-yl)-5-(4-((1-(2-(4- ((1R,2S)-6-hydroxy-2-phenyl-1,2,3,4- tetrahydronaphthalen-1- yl)phenoxy)ethyl)piperidin-4-yl)methyl)-3,5- dimethylpiperazin-1-yl)isoindoline-1,3-dione |
| | 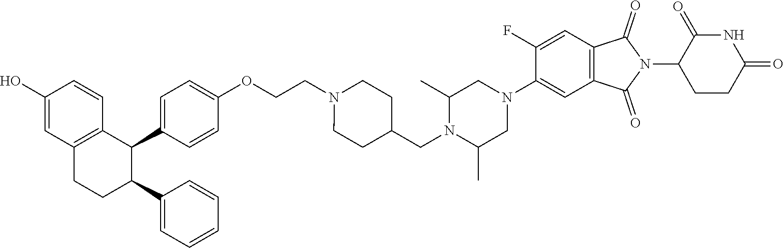 [SMILES: CC1CN(c2cc3c(cc2F)C(=O)N(C2CCC(=O)NC2=O)C3=O)CC(C)N1CC1CCN(CCOc2ccc([C@@H]3c4ccc(O)cc4CC[C@@H]3c3ccccc3)cc2)CC1] | 2-(2,6-dioxopiperidin-3-yl)-5-fluoro-6-(4-((1- (2-(4-((1R,2S)-6-hydroxy-2-phenyl-1,2,3,4- tetrahydronaphthalen-1- yl)phenoxy)ethyl)piperidin-4-yl)methyl)-3,5- dimethylpiperazin-1-yl)isoindoline-1,3-dione |
| | 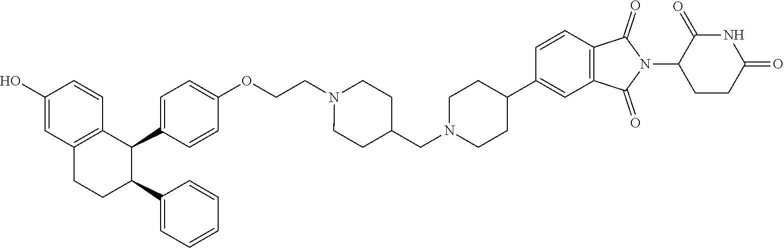 [SMILES: O=C1CCC(N2C(=O)c3ccc(C4CCN(CC5CCN(CCOc6ccc([C@@H]7c8ccc(O)cc8CC[C@@H]7c7ccccc7)cc6)CC5)CC4)cc3C2=O)C(=O)N1] | 2-(2,6-dioxopiperidin-3-yl)-5-(1-((1-(2-(4- ((1R,2S)-6-hydroxy-2-phenyl-1,2,3,4- tetrahydronaphthalen-1- yl)phenoxy)ethyl)piperidin-4- yl)methyl)piperidin-4-yl)isoindoline-1,3-dione |
| | 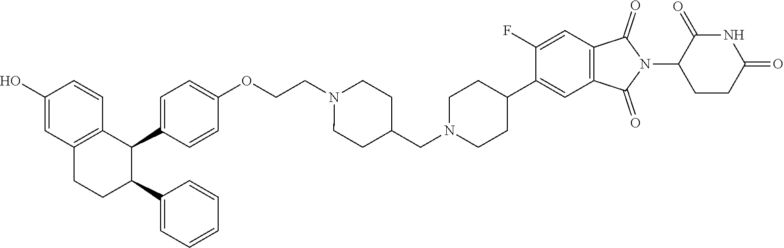 [SMILES: O=C1CCC(N2C(=O)c3cc(F)c(C4CCN(CC5CCN(CCOc6ccc([C@@H]7c8ccc(O)cc8CC[C@@H]7c7ccccc7)cc6)CC5)CC4)cc3C2=O)C(=O)N1] | 2-(2,6-dioxopiperidin-3-yl)-5-fluoro-6-(1-((1- (2-(4-((1R,2S)-6-hydroxy-2-phenyl-1,2,3,4- tetrahydronaphthalen-1 - yl)phenoxy)ethyl)piperidin-4- yl)methyl)piperidin-4-yl)isoindoline-1,3-dione |
| | 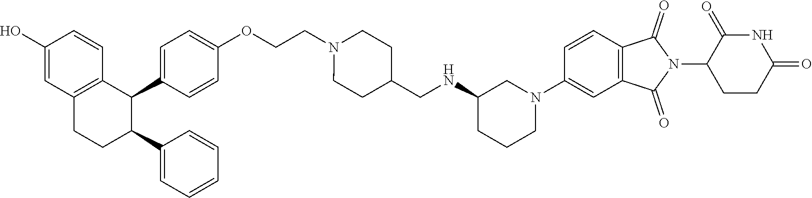 [SMILES: O=C1CCC(N2C(=O)c3ccc(N4CCC[C@@H](NCC5CCN(CCOc6ccc([C@@H]7c8ccc(O)cc8CC[C@@H]7c7ccccc7)cc6)CC5)C4)cc3C2=O)C(=O)N1] | 2-(2,6-dioxopiperidin-3-yl)-5-((R)-3-(((1-(2-(4- ((1R,2S)-6-hydroxy-2-phenyl-1,2,3,4- tetrahydronaphthalen-1- yl)phenoxy)ethyl)piperidin-4- yl)methyl)amino)piperidin-1-yl)isoindoline- 1,3-dione |
| | 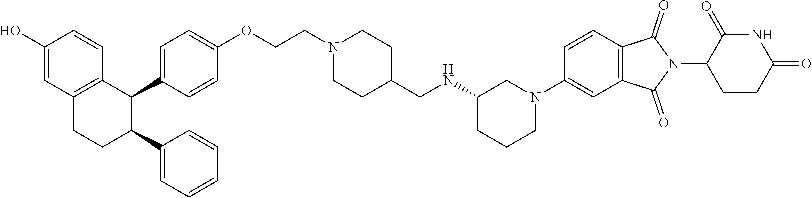 [SMILES: O=C1CCC(N2C(=O)c3ccc(N4CCC[C@H](NCC5CCN(CCOc6ccc([C@@H]7c8ccc(O)cc8CC[C@@H]7c7ccccc7)cc6)CC5)C4)cc3C2=O)C(=O)N1] | 2-(2,6-dioxopiperidin-3-yl)-5-((S)-3-(((1-(2-(4- ((1R,2S)-6-hydroxy-2-phenyl-1,2,3,4- tetrahydronaphthalen-1- yl)phenoxy)ethyl)piperidin-4- yl)methyl)amino)piperidin-1-yl)isoindoline- 1,3-dione |
| | 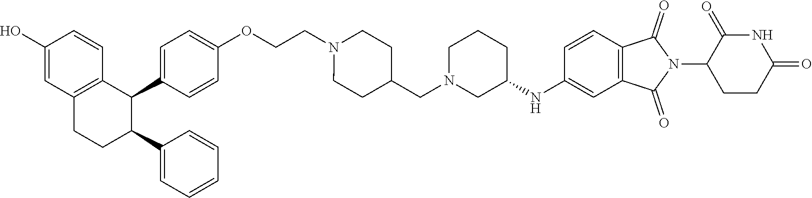 [SMILES: O=C1CCC(N2C(=O)c3ccc(N[C@H]4CCCN(CC5CCN(CCOc6ccc([C@@H]7c8ccc(O)cc8CC[C@@H]7c7ccccc7)cc6)CC5)C4)cc3C2=O)C(=O)N1] | 2-(2,6-dioxopiperidin-3-yl)-5-(((S)-1-((1-(2-(4- ((1R,2S)-6-hydroxy-2-phenyl-1,2,3,4- tetrahydronaphthalen-1- yl)phenoxy)ethyl)piperidin-4- yl)methyl)piperidin-3-yl)amino)isoindoline- 1,3-dione |
| | 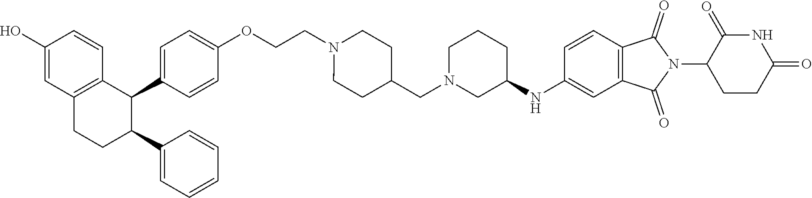 [SMILES: O=C1CCC(N2C(=O)c3ccc(N[C@@H]4CCCN(CC5CCN(CCOc6ccc([C@@H]7c8ccc(O)cc8CC[C@@H]7c7ccccc7)cc6)CC5)C4)cc3C2=O)C(=O)N1] | 2-(2,6-dioxopiperidin-3-yl)-5-(((R)-1-((1-(2-(4- ((1R,2S)-6-hydroxy-2-phenyl-1,2,3,4- tetrahydronaphthalen-1- yl)phenoxy)ethyl)piperidin-4- yl)methyl)piperidin-3-yl)amino)isoindoline- 1,3-dione |
| GT-03772 | 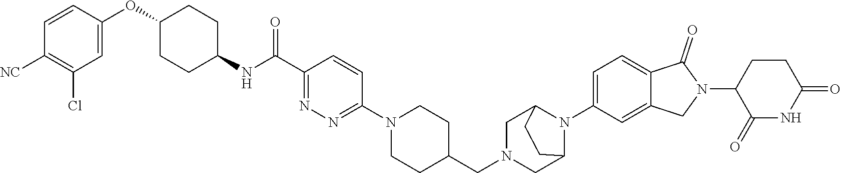 [SMILES: N#Cc1ccc(O[C@H]2CC[C@H](NC(=O)c3ccc(N4CCC(CN5CC6CCC(C5)N6c5ccc6c(c5)CN(C5CCC(=O)NC5=O)C6=O)CC4)nn3)CC2)cc1Cl] | N-((1r,4r)-4-(3-chloro-4- cyanophenoxy)cyclohexyl)-6-(4-((8-(2-(2,6- dioxopiperidin-3-yl)-1-oxoisoindolin-5-yl)-3,8- diazabicyclo[3.2.1]octan-3-yl)methyl)piperidin- 1-yl)pyridazine-3-carboxamide |
| GT-03886 | 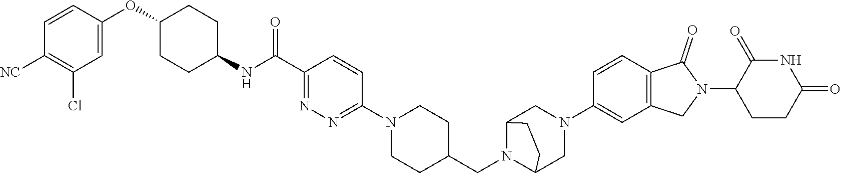 [SMILES: N#Cc1ccc(O[C@H]2CC[C@H](NC(=O)c3ccc(N4CCC(CN5C6CCC5CN(c5ccc7c(c5)CN(C5CCC(=O)NC5=O)C7=O)C6)CC4)nn3)CC2)cc1Cl] | N-((1r,4r)-4-(3-chloro-4- cyanophenoxy)cyclohexyl)-6-(4-((3-(2-(2,6- dioxopiperidin-3-yl)-1-oxoisoindolin-5-yl)-3,8- diazabicyclo[3.2.1]octan-8-yl)methyl)piperidin- 1-yl)pyridazine-3-carboxamide |
| GT-03518 | 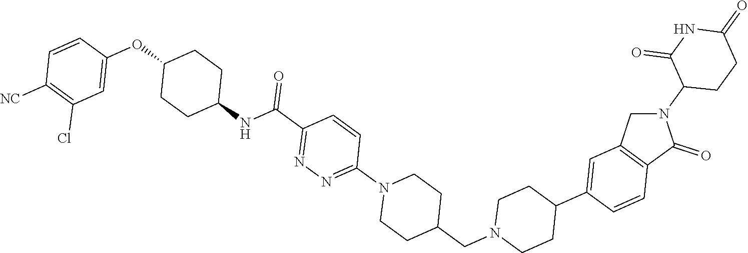 [SMILES: N#Cc1ccc(O[C@H]2CC[C@H](NC(=O)c3ccc(N4CCC(CN5CCC(c6ccc7c(c6)CN(C6CCC(=O)NC6=O)C7=O)CC5)CC4)nn3)CC2)cc1Cl] | N-((1r,4r)-4-(3-chloro-4- cyanophenoxy)cyclohexyl)-6-(4-((4-(2-(2,6- dioxopiperidin-3-yl)-1-oxoisoindolin-5- yl)piperidin-1-yl)methyl)piperidin-1- yl)pyridazine-3-carboxamide |
| GT-03519 | 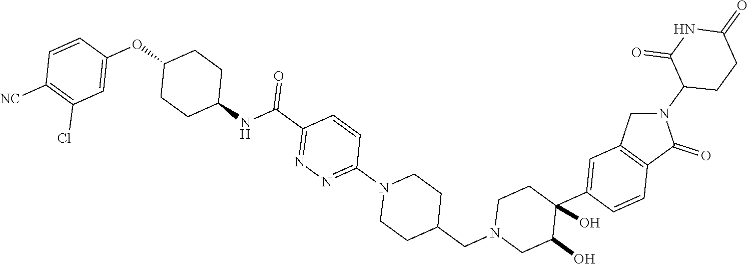 [SMILES: N#Cc1ccc(O[C@H]2CC[C@H](NC(=O)c3ccc(N4CCC(CN5CC[C@](O)(c6ccc7c(c6)CN(C6CCC(=O)NC6=O)C7=O)[C@@H](O)C5)CC4)nn3)CC2)cc1Cl] | N-((1r,4r)-4-(3-chloro-4- cyanophenoxy)cyclohexyl)-6-(4-(((3S,4S)-4- (2-(2,6-dioxopiperidin-3-yl)-1-oxoisoindolin-5- yl)-3,4-dihydroxypiperidin-1- yl)methyl)piperidin-1-yl)pyridazine-3- carboxamide |
| GT-03520 | 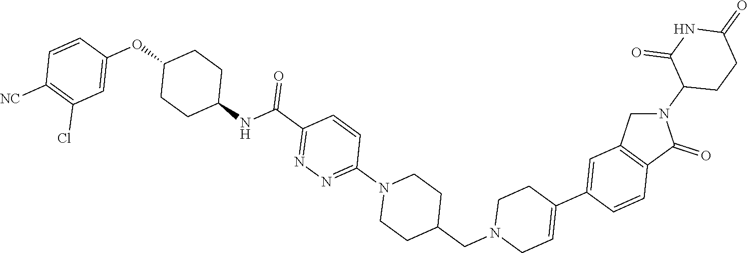 [SMILES: N#Cc1ccc(O[C@H]2CC[C@H](NC(=O)c3ccc(N4CCC(CN5CC=C(c6ccc7c(c6)CN(C6CCC(=O)NC6=O)C7=O)CC5)CC4)nn3)CC2)cc1Cl] | N-((1r,4r)-4-(3-chloro-4- cyanophenoxy)cyclohexyl)-6-(4-((4-(2-(2,6- dioxopiperidin-3-yl)-1-oxoisoindolin-5-yl)-3,6- dihydropyridin-1(2H)-yl)methyl)piperidin-1- yl)pyridazine-3-carboxamide |
| GT-03525 | 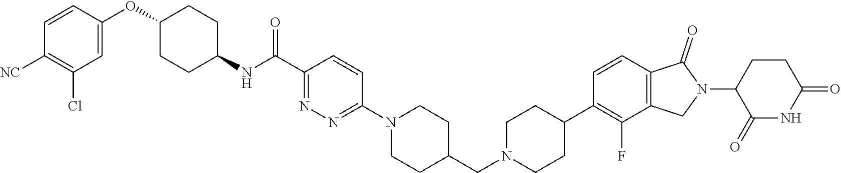 [SMILES: N#Cc1ccc(O[C@H]2CC[C@H](NC(=O)c3ccc(N4CCC(CN5CCC(c6ccc7c(c6F)CN(C6CCC(=O)NC6=O)C7=O)CC5)CC4)nn3)CC2)cc1Cl] | N-((1r,4r)-4-(3-chloro-4- cyanophenoxy)cyclohexyl)-6-(4-((4-(2-(2,6- dioxopiperidin-3-yl)-4-fluoro-1-oxoisoindolin- 5-yl)piperidin-1-yl)methyl)piperidin-1- yl)pyridazine-3-carboxamide |
| GT-03540 | 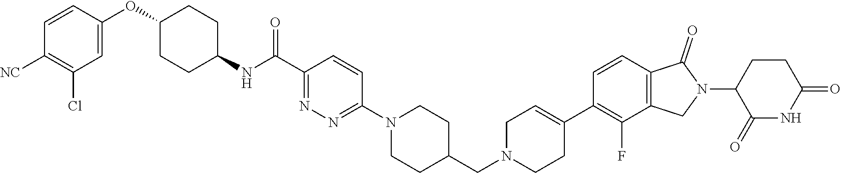 [SMILES: N#Cc1ccc(O[C@H]2CC[C@H](NC(=O)c3ccc(N4CCC(CN5CC=C(c6ccc7c(c6F)CN(C6CCC(=O)NC6=O)C7=O)CC5)CC4)nn3)CC2)cc1Cl] | N-((1r,4r)-4-(3-chloro-4- cyanophenoxy)cyclohexyl)-6-(4-((4-(2-(2,6- dioxopiperidin-3-yl)-4-fluoro-1-oxoisoindolin- 5-yl)-3,6-dihydropyridin-1(2H)- yl)methyl)piperidin-1-yl)pyridazine-3- carboxamide |
| GT-03580 | 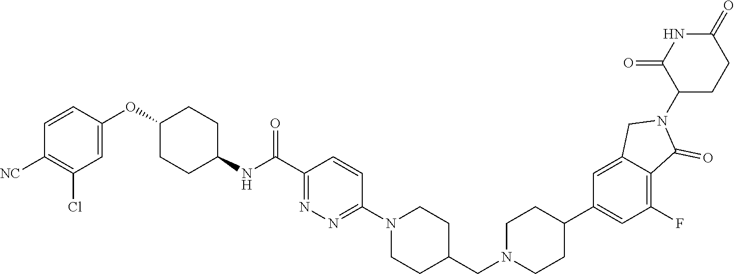 [SMILES: N#Cc1ccc(O[C@H]2CC[C@H](NC(=O)c3ccc(N4CCC(CN5CCC(c6cc(F)c7c(c6)CN(C6CCC(=O)NC6=O)C7=O)CC5)CC4)nn3)CC2)cc1Cl] | N-((1r,4r)-4-(3-chloro-4- cyanophenoxy)cyclohexyl)-6-(4-((4-(2-(2,6- dioxopiperidin-3-yl)-7-fluoro-1-oxoisoindolin- 5-yl)piperidin-1-yl)methyl)piperidin-1- yl)pyridazine-3-carboxamide |
| GT-03589 | 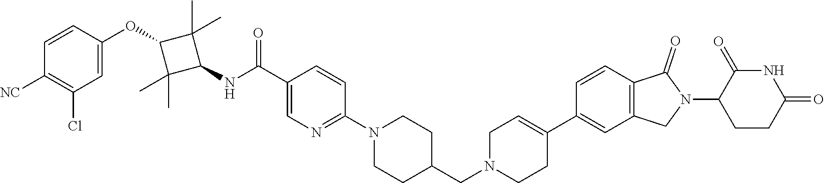 [SMILES: CC1(C)[C@H](NC(=O)c2ccc(N3CCC(CN4CC=C(c5ccc6c(c5)CN(C5CCC(=O)NC5=O)C6=O)CC4)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((4-(2-(2,6- dioxopiperidin-3-yl)-1-oxoisoindolin-5- yl)piperidin-1-yl)methyl)piperidin-1- yl)nicotinamide |
| GT-03590 | 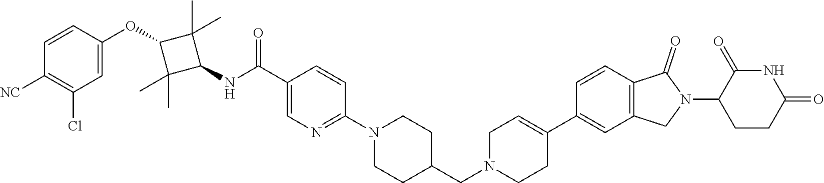 [SMILES: CC1(C)[C@H](NC(=O)c2ccc(N3CCC(CN4CC=C(c5ccc6c(c5)CN(C5CCC(=O)NC5=O)C6=O)CC4)CC3)nc2)C(C)(C)[C@H]1Oc1ccc(C#N)c(Cl)c1] | N-((1r,3r)-3-(3-chloro-4-cyanophenoxy)- 2,2,4,4-tetramethylcyclobutyl)-6-(4-((4-(2-(2,6- dioxopiperidin-3-yl)-1-oxoisoindolin-5-yl)-3,6- dihydropyridin-1(2H)-yl)methyl)piperidin-1- yl)nicotinamide |
| GT-03665 | 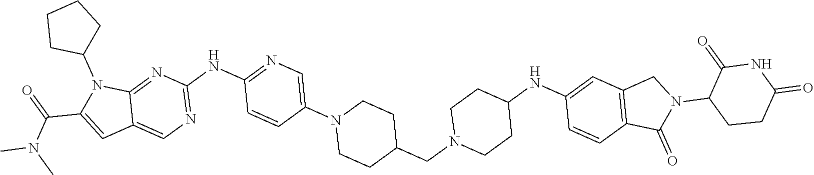 [SMILES: CN(C)C(=O)c1cc2cnc(Nc3ccc(N4CCC(CN5CCC(Nc6ccc7c(c6)CN(C6CCC(=O)NC6=O)C7=O)CC5)CC4)cn3)nc2n1C1CCCC1] | 7-cyclopentyl-2-((5-(4-((4-((2-(2,6- dioxopiperidin-3-yl)-1-oxoisoindolin-5- yl)amino)piperidin-1-yl)methyl)piperidin-1- yl)pyridin-2-yl)amino)-N,N-dimethyl-7H- pyrrolo[2,3-d]pyrimidine-6-carboxamide |
| GT-03533 | 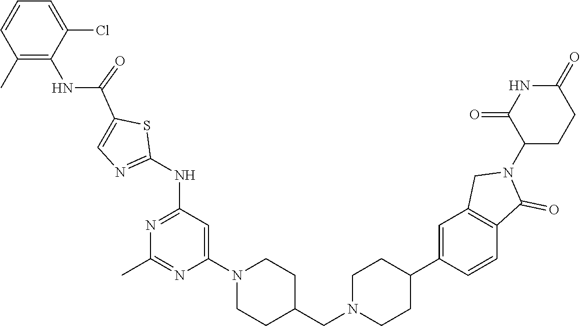 [SMILES: Cc1nc(Nc2ncc(C(=O)Nc3c(C)cccc3Cl)s2)cc(N2CCC(CN3CCC(c4ccc5c(c4)CN(C4CCC(=O)NC4=O)C5=O)CC3)CC2)n1] | N-(2-chloro-6-methylphenyl)-2-((6-(4-((4-(2- (2,6-dioxopiperidin-3-yl)-1-oxoisoindolin-5- yl)piperidin-1-yl)methyl)piperidin-1-yl)-2- methylpyrimidin-4-yl)amino)thiazole-5- carboxamide |
| GT-03534 | 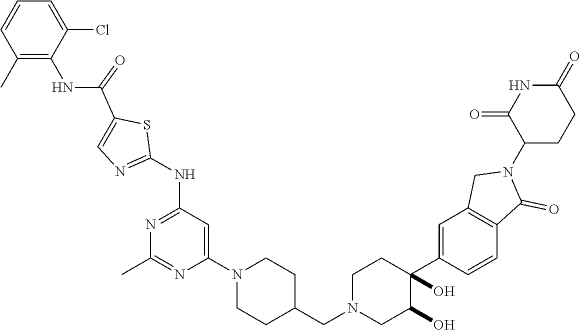 [SMILES: Cc1nc(Nc2ncc(C(=O)Nc3c(C)cccc3Cl)s2)cc(N2CCC(CN3CC[C@](O)(c4ccc5c(c4)CN(C4CCC(=O)NC4=O)C5=O)[C@@H](O)C3)CC2)n1] | N-(2-chloro-6-methylphenyl)-2-((6-(4- (((3S,4S)-4-(2-(2,6-dioxopiperidin-3-yl)-1- oxoisoindolin-5-yl)-3,4-dihydroxypiperidin-1- yl)methyl)piperidin-1-yl)-2-methylpyrimidin-4- yl)amino)thiazole-5-carboxamide |

### II. Other Forms of Compounds (Including Salts, Enantiomers, Stereoisomers, Solvates, Isotopically Enriched Analogs, Prodrugs, or Polymorphs of Compounds)

The compounds of the present disclosure have the structures of any one of Formula (I), Formula (II), Formula (III), Formula (III-1), Formula (III-2), Formula (III-3), Formula (III-4), Formula (III-5), Formula (III-6), Formula (III-7), Formula (III-8), Formula (III-9), Formula (III-10), Formula (III-11), Formula (III-12), Formula (III-13), Formula (III-14), Formula (III-15), Formula (III-16), Formula (III-17), Formula (III-18), Formula (III-19), Formula (III-20), Formula (III-21), Formula (III-22), Formula (III-23) or Formula (III-24). Unless otherwise specified, all references to the compounds of the present disclosure also include compounds of any one of Formula (I), Formula (II), Formula (III), Formula (III-1), Formula (III-2), Formula (III-3), Formula (III-4), Formula (III-5), Formula (III-6), Formula (III-7), Formula (III-8), Formula (III-9), Formula (III-10), Formula (III-11), Formula (III-12), Formula (III-13), Formula (III-14), Formula (III-15), Formula (III-16), Formula (III-17), Formula (III-18), Formula (III-19), Formula (III-20), Formula (III-21), Formula (III-22), Formula (III-23) or Formula (III-24) and specific compounds within the scope of these general formulae.

It should be recognized that compounds of the present disclosure (including Formula (I), Formula (II), Formula (III), Formula (III-1), Formula (III-2), Formula (III-3), Formula (III-4), Formula (III-5), Formula (III-6), Formula (III-7), Formula (III-8), Formula (III-9), Formula (III-10), Formula (III-11), Formula (III-12), Formula (III-13), Formula (III-14), Formula (III-15), Formula (III-16), Formula (III-17), Formula (III-18), Formula (III-19), Formula (III-20), Formula (III-21), Formula (III-22), Formula (III-23) and Formula (III-24)) may have a stereo-configuration and thus can exist in more than one stereoisomeric form. The present disclosure also relates to optically enriched compounds having a stereo-configuration, e.g., greater than about 90% enantiomeric/diastereomeric excess (“ee”), such as about 95% ee or 97% ee, or greater than 99% ee, and mixtures thereof, including racemic mixtures. As used herein, “optically enriched” means that a mixture of enantiomers consists of a significantly greater proportion of one enantiomer, and can be described by enantiomeric excess (ee %). Purification of isomers and separation of mixtures of isomers can be accomplished by standard techniques known in the art (e.g., column chromatography, preparative TLC, preparative HPLC, asymmetric synthesis (e.g., by using chiral intermediates) and/or or chiral resolution, etc.).

In some embodiments, polymorph forms or salts of the compounds of the present disclosure are also provided. Salts of the compounds of the present disclosure can be pharmaceutically acceptable salts including, but not limited to, hydrohalides (including hydrochlorides and hydrobromates), sulfates, citrates, maleates, sulfonates, citrates, lactates, L-tartrates, fumarates, L-malates, hippurates, D-glucuronates, glycolates, mucates, orotates, glycinates, alaninates, argininates, cinnamates, pamoates, benzenesulfonates, methanesulfonates, acetates, propionates, valerates, triphenylacetates, L-prolinates, ferulates, 2-hydroxyethanesulfonates, nitrates, gentisates, salicylates, glutarates, stearates, camphorsulfonates, phosphates, dihydrophosphates, pyrophosphates, metaphosphates, oxalates, malonates, benzoates, mandelates, succinates, trifluoroacetates, hydroxyacetates, or p-toluenesulfonates, etc. The compounds of the present disclosure can exist as non-solvated or solvated forms in pharmaceutically acceptable solvents such as water, ethanol, and the like. In some embodiments, compounds of the present disclosure can be prepared as prodrugs or precursor drugs. Prodrugs can be converted into parent drugs in the body to play their role. In some embodiments, isotopically-labeled compounds of the present disclosure are also provided, examples of which include deuterium (D or ²H).

### III. Compositions/Formulations

In some embodiments, the present disclosure provides a pharmaceutical composition comprising as active ingredient the compound of the present disclosure or a pharmaceutically acceptable salt, solvate, isotopically enriched analog, polymorph, prodrug, stereoisomer (including enantiomer), or mixture of stereoisomers thereof, and at least one pharmaceutically acceptable carrier.

In some embodiments, pharmaceutically acceptable carriers include, but are not limited to, fillers, stabilizers, dispersants, suspending agents, diluents, excipients, thickeners, colorants, solvents, or encapsulating materials. Each carrier must be “acceptable” in the sense of being compatible with the other ingredients of the formulation (including the compounds useful in the present disclosure) and not injurious to the patient. Some examples of materials that can be used as pharmaceutically acceptable carriers include: sugars such as lactose, glucose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository wax; oils such as peanut oil, cottonseed oil, safflower oil, sesame oil, olive oil, corn oil and soybean oil; glycols such as propylene glycol; polyols such as glycerol, sorbitol, mannitol, and polyethylene glycol; esters such as ethyl oleate and ethyl laurate; agar; buffers such as magnesium hydroxide and aluminum hydroxide; surfactant phosphate buffer solution; polyethylene oxide, polyvinylpyrrolidone, polyacrylamide, poloxamer; and other common non-toxic compatible substances used in pharmaceutical formulations.

The pharmaceutical composition of the present disclosure can further comprise at least one second therapeutic agent, e.g., an anticancer agent. The second therapeutic agent may be used in combination with the compounds of Formula (I), Formula (II) or Formula (III) described in the present disclosure to treat the diseases or disorders as disclosed herein. The second therapeutic agent includes, but is not limited to, chemotherapeutic agents, immunotherapeutic agents, gene therapy agents, and the like.

The pharmaceutical composition of the present disclosure comprising, as an active ingredient, the compounds of Formula (I), Formula (II) or Formula (III) of the present disclosure or a pharmaceutically acceptable salt thereof can be formulated into any suitable formulations such as sprays, patches, tablets (such as conventional tablets, dispersible tablets, orally disintegrating tablets), capsules (such as soft capsules, hard capsules, enteric-coated capsules), dragees, troches, powders, granules, powder injections, suppositories, or liquid formulations (such as suspensions (e.g., aqueous or oily suspensions), solutions, emulsions, or syrups), or conventional injection dosage forms such as injectable solutions (e.g., sterile injectable solutions formulated according to methods known in the art using water, Ringer's solution, or isotonic sodium chloride solution or the like as a vehicle or solvent) or lyophilized injectable formulation and the like, depending upon a suitable route of administration (including, but not limited to, nasal administration, inhalation administration, topical administration, oral administration, oral mucosal administration, rectal administration, intrapleural administration, intraperitoneal administration, vaginal administration, intramuscular administration, subcutaneous administration, transdermal administration, epidural administration, intrathecal administration, and intravenous administration). Those skilled in the art can also formulate the compounds of Formula (I), Formula (II) or Formula (III) of the present disclosure into conventional, dispersible, chewable, orally disintegrating or rapidly dissolving formulations, or sustained-release capsules or controlled-release capsules as needed.

### IV. Kits/Packaged Products

The compounds of Formula (I), Formula (II) or Formula (III) or a pharmaceutically acceptable salt, solvate, isotopically enriched analog, polymorph, prodrug, stereoisomer (including enantiomer), or mixture of stereoisomers thereof is used as a medicament. The medicament of the present disclosure or the pharmaceutical composition of the present disclosure may be presented in a kit/packaged product. The kit/packaged product may include a package or container including, but not limited to, ampoules, blister packs, pharmaceutical plastic bottles, vials, pharmaceutical glass bottles, containers, syringes, laminated flexible packaging, co-extruded film infusion containers, test tubes and dispensing devices, and the like. The kit/packaged product may contain instructions for use of the product.

### V. Methods and Uses

The compounds of Formula (I) or Formula (II) or a pharmaceutically acceptable salt, solvate, isotopically enriched analog, polymorph, prodrug, stereoisomer (including enantiomer), or mixture of stereoisomers thereof can be used as a medicament. Especially the compounds of Formula (I) or Formula (II) or a pharmaceutically acceptable salt, solvate, isotopically enriched analog, polymorph, prodrug, stereoisomer (including enantiomer), or mixture of stereoisomers thereof can be used for the manufacture of a medicament for the prevention and/or treatment of a cereblon protein-mediated disease or disorder.

The present disclosure provides a method for preventing and/or treating a cereblon protein-mediated disease or disorder in a subject, comprising administering to the subject a therapeutically effective amount of the compounds of Formula (I) or Formula (II) of the present disclosure or a pharmaceutically acceptable salt thereof, or the pharmaceutical composition of the present disclosure comprising as an active ingredient the compounds of Formula (I) or Formula (II) of the present disclosure or a pharmaceutically acceptable salt thereof. In some embodiments, the cereblon protein-mediated disease or disorder comprises: tumor, infectious disease, autoinflammatory disease, inflammatory disease, autoimmune disease, neurological disease, respiratory disease, anemia, hemorrhagic shock, transplant rejection, multiple organ dysfunction syndrome (MODS), sarcoidosis, adult respiratory distress syndrome, congestive heart failure, myocardial infarction, Unverricht's syndrome, Richter syndrome (RS), acute liver failure, or diabetes. In some embodiments, the cereblon protein-mediated disease or disorder includes, but is not limited to, myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including acute myeloid leukemia, chronic myelogenous leukemia, B-cell chronic lymphocytic leukemia, leukemia-associated anemia, acute myeloid leukemia (AML); lymphoma, including diffuse large B-cell lymphoma, non-Hodgkin's lymphoma, anaplastic lymphoma, anaplastic large cell lymphoma, CD20 positive lymphoma, mantle cell lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; thyroid cancer; melanoma; lung cancer; inflammatory myofibroblastoma; colorectal cancer; brain glioma; astroblastoma; ovarian cancer; bronchial cancer; prostate cancer; breast cancer, including triple negative breast cancer, sporadic breast cancer and patients with Cowden syndrome; pancreatic cancer; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Unverricht's syndrome; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; autoinflammatory diseases, including gout, etc; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc.; septic shock; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; congestive heart failure; myocardial infarction; multiple organ dysfunction caused by cachexia and septic shock; and acute liver failure.

In the method for preventing and/or treating a cereblon protein-mediated disease or disorder in a subject, the compound of Formula (I) or Formula (II) of the present disclosure or the pharmaceutical composition comprising as an active ingredient the compound of Formula (I) or Formula (II) of the present disclosure of the present disclosure is administered to the subject through at least one mode of administration selected from the group consisting of nasal administration, inhalation administration, topical administration, oral administration, oral mucosal administration, rectal administration, pleural cavity administration, peritoneal administration, vaginal administration, intramuscular administration, subcutaneous, transdermal, epidural, intrathecal, and intravenous administration.

The compound of Formula (III) or a pharmaceutically acceptable salt, solvate, isotopically enriched analog, polymorph, prodrug, stereoisomer (including enantiomer), or mixture of stereoisomers thereof can be used as a medicament. Especially the compound of Formula (III) or a pharmaceutically acceptable salt, solvate, isotopically enriched analog, polymorph, prodrug, stereoisomer (including enantiomer), or mixture of stereoisomers thereof can be used for the manufacture of a medicament for the prevention and/or treatment of a disease or disorder selected from the group consisting of tumor, infectious disease, autoinflammatory disease, inflammatory disease, autoimmune disease, neurological disease, respiratory disease, anemia, hemorrhagic shock, transplant rejection, multiple organ dysfunction syndrome (MODS), sarcoidosis, adult respiratory distress syndrome, congestive heart failure, myocardial infarction, Unverricht's syndrome, Richter syndrome (RS), acute liver failure, diabetes, Kennedy disease, seborrheic alopecia, hirsutism, skin disease, cardiovascular disease, dysfunctional uterine bleeding, anemia, pediatric aplastic anemia, endometriosis, transplant rejection, polycystic ovary syndrome, and thyroid disease. In some embodiments, the disease or disorder includes, but is not limited to, myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including acute myeloid leukemia, chronic myelogenous leukemia, B-cell chronic lymphocytic leukemia, leukemia-associated anemia, acute myeloid leukemia (AML); lymphoma, including diffuse large B-cell lymphoma, non-Hodgkin's lymphoma, anaplastic lymphoma, anaplastic large cell lymphoma, CD20 positive lymphoma, mantle cell lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; thyroid cancer; melanoma; lung cancer, including lung adenocarcinoma, lung squamous cell carcinoma; inflammatory myofibroblastoma; colorectal cancer; brain glioma; astroblastoma; ovarian cancer; bronchial cancer; prostate cancer; breast cancer, including triple negative breast cancer, sporadic breast cancer and patients with Cowden syndrome; pancreatic cancer; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Unverricht's syndrome; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; autoinflammatory diseases, including gout, etc; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc; septic shock; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; multiple organ dysfunction caused by cachexia and septic shock; acute liver failure; dysfunctional uterine bleeding; anemia; pediatric aplastic anemia; endometriosis; polycystic ovary syndrome; thyroid disease; cardiovascular diseases (e.g., coronary heart disease, congestive heart failure, myocardial infarction, atherosclerosis); skin diseases (e.g., acne); Kennedy disease; seborrheic alopecia; and hirsutism.

The present disclosure provides a method for preventing and/or treating a disease or disorder in a subject, comprising administering to the subject a therapeutically effective amount of the compound of Formula (III) of the present disclosure or a pharmaceutically acceptable salt thereof, or the pharmaceutical composition of the present disclosure comprising as an active ingredient the compound of Formula (III) of the present disclosure or a pharmaceutically acceptable salt thereof, wherein the disease or disorder includes, but is not limited to, tumor, infectious disease, autoinflammatory disease, inflammatory disease, autoimmune disease, neurological disease, respiratory disease, anemia, hemorrhagic shock, transplant rejection, multiple organ dysfunction syndrome (MODS), sarcoidosis, adult respiratory distress syndrome, congestive heart failure, myocardial infarction, Unverricht's syndrome, Richter syndrome (RS), acute liver failure, diabetes, Kennedy disease, seborrheic alopecia, hirsutism, skin disease, cardiovascular disease, dysfunctional uterine bleeding, anemia, pediatric aplastic anemia, endometriosis, transplant rejection, polycystic ovary syndrome, and thyroid disease. In some embodiments, the disease or disorder includes, but is not limited to, myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including acute myeloid leukemia, chronic myelogenous leukemia, B-cell chronic lymphocytic leukemia, leukemia-associated anemia, acute myeloid leukemia (AML); lymphoma, including diffuse large B-cell lymphoma, non-Hodgkin's lymphoma, anaplastic lymphoma, anaplastic large cell lymphoma, CD20 positive lymphoma, mantle cell lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; thyroid cancer; melanoma; lung cancer, including lung adenocarcinoma, lung squamous cell carcinoma; inflammatory myofibroblastoma; colorectal cancer; brain glioma; astroblastoma; ovarian cancer; bronchial cancer; prostate cancer; breast cancer, including triple negative breast cancer, sporadic breast cancer and patients with Cowden syndrome; pancreatic cancer; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Unverricht's syndrome; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; autoinflammatory diseases, including gout, etc.; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc; septic shock; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; multiple organ dysfunction caused by cachexia and septic shock; acute liver failure; dysfunctional uterine bleeding; anemia; pediatric aplastic anemia; endometriosis; polycystic ovary syndrome; thyroid disease; cardiovascular diseases (e.g., coronary heart disease, congestive heart failure, myocardial infarction, atherosclerosis); skin diseases (e.g., acne); Kennedy disease; seborrheic alopecia; and hirsutism.

The term “treatment” or “treating” refers to the administration of the compound of Formula (I), Formula (II) or Formula (III) or a pharmaceutically acceptable salt thereof according to the present disclosure, or the pharmaceutical composition containing, as an active ingredient, the compound of Formula (I), Formula (II) or Formula (III) or a pharmaceutically acceptable salt thereof, to a subject to mitigate (alleviate) undesirable diseases or conditions, such as the development of a cancer or tumor. The beneficial or desired clinical results of the present disclosure include, but are not limited to: alleviating symptoms, reducing the severity of the disease, stabilizing the state of the disease, slowing down or delaying the progression of the disease, improving or alleviating the condition, and alleviating the disease.

A “therapeutic effective amount” of the compound of the present disclosure depends on a variety of factors, including the activity of the specific compound used, the metabolic stability of the compound and the duration of its action, the age, sex and weight of the patient, the patient's current medical condition, the route and duration of administration, the excretion rate, the combined administration of additional drugs, and the progression of the diseases or conditions of the patient being treated. Those skilled in the art will be able to determine appropriate dosages based on these and other factors.

It is to be understood that the choice of using one or more active compounds and/or compositions and their dosage depends on the basic situations of the individual (which should generally render the individual situation to achieve the best effect). Dosing and dosing regimens should be within the ability of those skilled in the art, and the appropriate dosage depends on many factors including the knowledge and ability of the physicians, veterinarians or researchers (Jun Li (chief editor), “Clinical Pharmacology”, 4th edition, People's Public Health Press, 2008).

As used herein, the term “patient” or “subject” to be treated refers to animal, for example mammal, including but not limited to primate (such as human being), cow, sheep, goat, horse, dog, cat, rabbit, guinea pig, rat, mice, etc.

The compound of Formula (I) or Formula (II) or a pharmaceutically acceptable salt, solvate, isotopically enriched analog, polymorph, prodrug, stereoisomer (including enantiomer), or mixture of stereoisomers thereof of the present disclosure can be used to prepare the compound of Formula (III) of the present disclosure. The compound of Formula (III) of the present disclosure can be used to treat and/or prevent a disease or disorder selected from the group consisting of tumor, infectious disease, autoinflammatory disease, inflammatory disease, autoimmune disease, neurological disease, respiratory disease, anemia, hemorrhagic shock, transplant rejection, multiple organ dysfunction syndrome (MODS), sarcoidosis, adult respiratory distress syndrome, congestive heart failure, myocardial infarction, Unverricht's syndrome, Richter syndrome (RS), acute liver failure, diabetes, Kennedy disease, seborrheic alopecia, hirsutism, skin disease, cardiovascular disease, dysfunctional uterine bleeding, anemia, pediatric aplastic anemia, endometriosis, transplant rejection, polycystic ovary syndrome, and thyroid disease.

### VI. Definitions

Unless otherwise specified, the following words, phrases and symbols used herein generally have the meanings as described below.

In general, the nomenclature used herein (including the IUPAC nomenclature) and the laboratory procedures described below (including those used in cell culture, organic chemistry, analytical chemistry, and pharmacology, etc.) are those well-known and commonly used in the art. Unless otherwise defined, all scientific and technical terms used herein in connection with the present disclosure described herein have the same meaning as commonly understood by one skill in the art. In addition, the use of the word “a” or “an” when used in conjunction with the term “comprising” or a noun in the claims and/or the specification may mean “one”, but it is also consistent with the meaning of “one or more”, “at least one”, and “one or more than one”. Similarly, the terms “another” or “other” can mean at least a second or more.

It should be understood that whenever the term “comprise” or “include” is used herein to describe various aspects, other similar aspects described by “consisting of” and/or “consisting essentially of” are also provided.

As used herein, the term “about” used alone or in combination refers to approximately, roughly, nearly, or around. When the term “about” is used in conjunction with a numerical range, it modifies that range by extending the boundaries above and below the stated numerical value. In general, the term “about” can modify a numerical value above and below the stated value by an upward or downward (increasing or decreasing) variation, e.g., 10%, 5%, 2%, or 1%.

As used herein, the wording “ . . . represents a bond” used alone or in combination means that the referenced group is a bond linker (that is, the referenced group is absent). For example, the wording “U represents a bond” means that U is a bond linker. In other words, when U represents a bond, the group R in the structure of Formula (I) is directly connected to phenyl ring in the structure of Formula (I). For example, the wording “R¹² represents a bond” means that R¹² is a bond linker. In other words, when R¹² represents a bond, the ring carbon atom connected to R¹² in the structure of Formula (PBM-7) is directly connected to R¹ of the compound of Formula (III).

As used herein, the term “inserted” of the expression “one or more groups Rₑ and/or one or more groups R_{f} and/or any combination of one or more groups Rₑ and R_{f} are inserted into the backbone carbon chain of the linear or branched C₂₋₃₀ alkylene group”, used alone or in combination, has a known definition in the art, which can mean that carbon-carbon bond between one or more pairs of adjacent carbon atoms in the referenced backbone carbon chain is interrupted by the groups Rₑ, R_{f}, or a combination of Rₑ and R_{f}. Herein, examples of the above-mentioned expression “one or more groups . . . are inserted into” may include, but are not limited to, that one or more (1-30, 1-20, or 1-15, 1-10, 1-9, 1-8, 1-7, 1-6, 1-5, 1-4, 1-3, or 1-2, or 1) groups Rₑ as defined herein and/or one or more (1-30, 1-20, or 1-15, 1-10, 1-9, 1-8, 1-7, 1-6, 1-5, 1-4, 1-3, or 1-2, or 1) groups R_{f} as defined herein and/or one or more (1-30, 1-20, or 1-15, 1-10, 1-9, 1-8, 1-7, 1-6, 1-5, 1-4, 1-3, or 1-2 T, or 1) combinations of Rₑ with R_{f} are inserted into the backbone carbon chain, and the resulting backbone chain group conforms to the covalent bond theory. For example, the expression “one or more groups Rₑ and/or one or more groups R_{f} and/or any combination of one or more groups Rₑ and R_{f} are inserted into the backbone carbon chain of the linear or branched C₂₋₃₀ alkylene group” can refer to that one or more (e.g., 1-30, 1-20, 1-15, 1-10, 1-8, 1-7, 1-6, 1-5, 1-4, 1-3, 1-2, or 1) groups Rₑ and/or R_{f} and/or one or more combinations of Rₑ with R_{f} are inserted between one or more pairs of any two adjacent carbon atoms of the backbone carbon chain of the linear or branched C₂₋₃₀ alkylene group, resulting in the formation of a backbone chain group containing one or more (e.g., 1-30, 1-20, 1-15, 1-10, 1-8, 1-7, 1-6, 1-5, 1-4, 1-3, 1-2, or 1) fragments “—CH₂—Rₑ—CH₂—” and/or one or more (e.g., 1-30, 1-20, 1-15, 1-10, 1-8, 1-7, 1-6, 1-5, 1-4, 1-3, 1-2, or 1) fragments “—CH₂—R_{f}—CH₂—” and/or one or more (e.g., 1-30, 1-20, 1-15, 1-10, 1-8, 1-7, 1-6, 1-5, 1-4, 1-3, 1-2, or 1) fragments “—CH₂—Rₑ—R_{f}—CH₂—”, where each Rₑ are the same or different, each R_{f} are the same or different, and are as defined herein.

Herein, it should be understood that the expression “one or more groups Rₑ and/or one or more groups R_{f} or any combination of one or more groups Rₑ and R_{f} are optionally inserted into the backbone carbon chain of the linear or branched C₂₋₃₀ alkylene group” includes embodiments where “one or more groups Rₑ and/or one or more groups R_{f} or any combination of one or more groups Rₑ and R_{f} are inserted into the backbone carbon chain of the linear or branched C₂₋₃₀ alkylene group”, as well as embodiments where “no one or more groups Rₑ and/or one or more groups R_{f} or any combination of one or more groups Rₑ and R_{f} are inserted into the backbone carbon chain of the linear or branched C₂₋₃₀ alkylene group”.

Herein, a bond interrupted by a wavy line shows the point of attachment of the depicted group to the rest of the molecule. For example, the group of Formula (ULM) depicted below

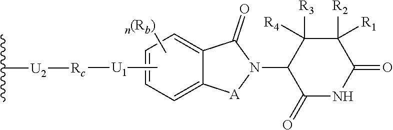

shows the point of attachment of U₂ in said group to LIN of the compound of Formula (III).

As used herein, the expression “a hydrogen atom of one or more CH₂ of the linear or branched C_{x-y} alkylene is replaced by . . . ”, used alone or in combination, means that a hydrogen atom of any one or more CH₂ of the linear or branched C_{x-y} alkylene is replaced by a substituent(s) as defined herein. Herein, the term “one or more” of “a hydrogen atom of one or more CH₂ of groups #-U—CH₂—, #-U—(CH₂)₂—, #-U—(CH₂)₃—, #-U—(CH₂)₄—, #-U—(CH₂)₅—, #-U—(CH₂)₆—, #-U—(CH₂)₇—, #-U—(CH₂)₈—, #-U—(CH₂)₉—, #-U—(CH₂)₁₀—, #-U—(CH₂)₁₁—, #-U—(CH₂)₁₂—, #-U—(CH₂)₁₃—, #-U—(CH₂)₁₄—, #-U—(CH₂)₁₅—, #-U—(CH₂)₁₆—, #-U—(CH₂)₁₇—, #-U—(CH₂)₁₈—, #-U—(CH₂)₁₉—, #-U—(CH₂)₂₀—, #-U—(CH₂)₂₁—, #-U—(CH₂)₂₂—, #-U—(CH₂)₂₅—, or #-U—(CH₂)₃₀—” may refer to part or all hydrogen atoms of each referenced alkylene group, including but not limited to 1-60 hydrogens. In some embodiments, the expression “a hydrogen atom of one or more CH₂” may refer to part or all of the hydrogen atoms of the referenced alkylene group, including but not limited to 1-30, such as 1-25, 1-20, 1-15, 1-10, 1-5, 1-4, 1-3, 1-2 or 1 hydrogen atoms. In some embodiments, the expression “a hydrogen atom of one or more CH₂” may include 1-3 of the plurality of hydrogen atoms of the referenced alkylene group. The number of hydrogens to be replaced is in principle not limited in any way, or is automatically limited by the size of the building unit.

As used herein, the wordings “optionally substituted” and “unsubstituted or substituted” can be used interchangeably. The term “substituted” usually means that one or more hydrogen atoms in the referenced structure are replaced by the same or different specific substituents.

As used herein, the term “oxo” or “oxo group” refers to ═O.

As used herein, the term “nicotinoyl” refers to

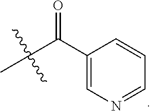 [SMILES: *C(=O)c1cccnc1]

As used herein, the term “halogen atom” or “halogen”, used alone or in combination, refers to fluorine, chlorine, bromine, or iodine.

As used herein, the term “alkyl”, used alone or in combination, refers to a linear or branched alkyl group. The term “Cₓ-C_{y} alkyl” or “C_{x-y} alkyl” (x and y each being an integer) refers to a linear or branched alkyl group containing from x to y carbon atoms. The term “C₁₋₁₀ alkyl” used alone or in combination in the present disclosure refers to a linear or branched alkyl group containing from 1 to 10 carbon atoms. Non-limiting examples of the C₁₋₁₀ alkyl of the present disclosure may include a C₁₋₉ alkyl, C₁₋₈ alkyl, C₂₋₈ alkyl, C₁₋₇ alkyl, C₁₋₆ alkyl, C₁₋₅ alkyl, and C₁₋₄ alkyl. Representative examples include methyl, ethyl, n-propyl, isopropyl, n-butyl, isobutyl, sec-butyl, tert-butyl, pentyl, isopentyl, neopentyl, tert-pentyl, hexyl, heptyl, octyl, nonyl, and decyl. The term “C₁₋₃ alkyl” or “C₁-C₃ alkyl” in the present disclosure refers to an alkyl group containing from 1 to 3 carbon atoms, and representative examples thereof include methyl, ethyl, n-propyl, and isopropyl. In the present disclosure, the “alkyl” is optionally substituted with one or more substituents optionally selected from the group consisting of halogen, hydroxyl, cyano, C₁₋₃ alkyl, C₁₋₃ alkoxy, trifluoromethyl, heterocyclyl, or a combination thereof.

As used herein, the term “halogenated alkyl” or “haloalkyl”, used alone or in combination, refers to a linear or branched alkyl group substituted with one or more halogens, wherein one or more hydrogen atom(s) of the alkyl group are replaced with one or more halogens. The term “halogenated C_{x-Cy} alkyl” or “halogenated C_{x-y} alkyl” (x and y are each an integer) refers to a linear or branched alkyl containing from x to y carbon atoms substituted with one or more halogens. The term “halogenated C₁₋₁₀ alkyl” used alone or in combination in the present invention refers to a linear or branched alkyl group containing from 1 to 10 carbon atoms substituted with one or more halogens. Examples of the halogenated C₁₋₁₀ alkyl group of the present disclosure include halogenated C₁₋₉ alkyl group, e.g., halogenated C₁₋₈ alkyl group, halogenated C₂₋₈ alkyl group, halogenated C₁₋₇ alkyl group, halogenated C₁₋₆ alkyl, halogenated C₁₋₅ alkyl, or halogenated C₁₋₄ alkyl. Representative examples include halomethyl, haloethyl, halo-n-propyl, haloisopropyl, halo-n-butyl, haloisobutyl, halo-sec-butyl, halo-tert-butyl, halopentyl, haloisoamyl, haloneopentyl, halo-tert-pentyl, halohexyl, haloheptyl, halooctyl, halononyl, and halodecyl. The term “halo-C₁₋₃ alkyl” or “halo-C₁₋C₃ alkyl” of the present disclosure refers to an alkyl group containing from 1 to 3 carbon atoms substituted with one or more halogens, and its representative examples include halomethyl, haloethyl, halo-n-propyl and haloisopropyl.

As used herein, the term “deuterated alkyl”, used alone or in combination, refers to a linear or branched alkyl group substituted with one or more deuterium atoms, wherein one or more hydrogen atom(s) of the alkyl group are replaced with one or more deuterium atoms. The term “deuterated C_{x-Cy} alkyl” or “deuterated C_{x-y} alkyl” (x and y are each an integer) refers to a linear or branched alkyl containing from x to y carbon atoms substituted with one or more deuterium atoms. The term “deuterated C₁₋₁₀ alkyl” used alone or in combination in the present invention refers to a linear or branched alkyl group containing from 1 to 10 carbon atoms substituted with one or more deuterium atoms. Examples of the deuterated C₁₋₁₀ alkyl group of the present disclosure include deuterated C₁₋₉ alkyl group, e.g., deuterated C₁₋₈ alkyl group, deuterated C₂₋₈ alkyl group, deuterated C₁₋₇ alkyl group, deuterated C₁₋₆ alkyl, deuterated C₁₋₅ alkyl, or deuterated C₁₋₄ alkyl. Representative examples include perdeuterated methyl (CD₃), perdeuterated ethyl (CD₃CD₂), perdeuterated n-propyl, perdeuterated isopropyl, perdeuterated n-butyl, perdeuterated isobutyl, perdeuterated sec-butyl, perdeuterated tert-butyl, perdeuterated pentyl, perdeuterated isopentyl, perdeuterated neopentyl, perdeuterated tert-pentyl, perdeuterated hexyl, perdeuterated heptyl, perdeuterated octyl, perdeuterated nonyl, and perdeuterated decyl. The term “deuterated C₁₋₃ alkyl” or “deuterated C₁-C₃ alkyl” of the present disclosure refers to an alkyl group containing from 1 to 3 carbon atoms substituted with one or more deuterium atoms, and its representative examples include perdeuterated methyl (CD₃) and perdeuterated ethyl (CD₃CD₂).

As used herein, the term “alkylene” (which is used interchangeably with “alkylene chain”), used alone or in combination, refers to a linear or branched divalent saturated hydrocarbon group composed of carbon and hydrogen atoms. The term “Cₓ-C_{y} alkylene” or “C_{x-y} alkylene” (x and y each being an integer) refers to a linear or branched alkylene group containing from x to y carbon atoms. Examples of the C₁-C₃₀ alkylene in the present disclosure may include C₁-C₃₀ alkylene, C₁-C₂₉ alkylene, C₁-C₂₈ alkylene, C₁-C₂₇ alkylene, C₁-C₂₆ alkylene, C₁-C₂₅ alkylene, C₁-C₂₄ alkylene, C₁-C₂₃ alkylene, C₁-C₂₂ alkylene, C₁-C₂₁ alkylene, C₁-C₂₀ alkylene, C₁-C₁₉ alkylene, C₁-C₁₈ alkylene, C₁-C₁₇ alkylene, C₁-C₁₆ alkylene, C₁-C₁₅ alkylene, C₁-C₁₄ alkylene, C₁-C₁₃ alkylene, C₁-C₁₂ alkylene, C₁-C₁₁ alkylene, C₁-C₁₀ alkylene, C₁-C₉ alkylene, C₁-C₈ alkylene, C₁-C₇ alkylene, C₁-C₆ alkylene, C₁-C₅ alkylene, C₁-C₄ alkylene, C₁-C₃ alkylene, or C₁-C₂ alkylene. Representative examples include, but are not limited to, methylene, ethylene, propylene, isopropylene, butylene, isobutylene, sec-butylene, tert-butylene, n-pentylene, isopentylene, neopentylidene, tert-pentylene, hexylene, heptylene, octylene, nonylene, decylene, undecylene, dodecylene, tridecylene, tetradecylene, pentadecylene, hexadecylene, heptadecylene, octadecylene, nonadecylene, eicosylene, heneicosylene, docosylene, tricosylene, tetracosylene, pentacosylene, hexacosylene, heptacosylene, octacosylene, nonacosylene, and triacontylene. In the present disclosure, the “alkylene” is optionally substituted with one or more substituents optionally selected from C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “alkoxy”, used alone or in combination, refers to a linear or branched alkoxy group having structural formula of —O-alkyl. Optionally, the alkyl portion of the alkoxy group may contain 1-10 carbon atoms. Representative examples of “alkoxy” include, but are not limited to, methoxy, ethoxy, propoxy, isopropoxy, n-butoxy, isobutoxy, tert-butoxy, pentyloxy, 2-pentyloxy, isopentyloxy, neopentyloxy, hexyloxy, 2-hexyloxy, 3-hexyloxy, 3-methylpentyloxy, etc. The term “C₁-C₃ alkoxy” or “C₁₋₃ alkoxy” refers to a linear or branched alkoxy group containing from 1 to 3 carbon atoms. Representative examples of C₁₋₃ alkoxy include, but are not limited to, methoxy, ethoxy, n-propoxy, and isopropoxy.

As used herein, the term “alkylamino”, used alone or in combination, refers to a linear or branched alkylamino group having structural formula of alkyl-NH—. Optionally, the alkyl portion of the alkylamino group may contain 1-10 carbon atoms. Representative examples of “alkylamino” include, but are not limited to, methyl-NH—, ethyl-NH—, n-propyl-NH—, isopropyl-NH—, n-butyl-NH—, isobutyl-NH—, tert-butyl-NH—, pentyl-NH—, and hexyl-NH—, etc. The term “C₁-C₃ alkyl-NH—” or “C₁₋₃ alkyl-NH—” refers to a linear or branched alkyl-NH— group containing from 1 to 3 carbon atoms. Representative examples of C₁₋₃ alkyl-NH— include, but are not limited to, methyl-NH—, ethyl-NH—, n-propyl-NH—, and isopropyl-NH—.

As used herein, the term “amino-substituted alkylene”, used alone or in combination, refers to a linear or branched alkylene group substituted with amino having structural formula of NH₂-alkylene. Optionally, the alkylene portion of the amino-substituted alkylene group may contain 1-10 carbon atoms. The term “amino-substituted C₁₋₃ alkylene” or “NH₂—C₁₋₃ alkyl” refers to a linear or branched alkylene group containing from 1 to 3 carbon atoms which is substituted with amino. Representative examples of amino-substituted C₁₋₃ alkylene include, but are not limited to, NH₂—CH₂—, NH₂—CH₂CH₂—, and NH₂—CH₂CH₂CH₂—.

As used herein, the term “alkyl-NHC(O)—”, used alone or in combination, refers to a linear or branched alkyl-NHC(O)— group having structural formula of alkyl-NHC(O)—. Optionally, the alkyl portion of the alkyl-NHC(O)— group may contain 1-10 carbon atoms. The term “C₁₋₃ alkyl-NHC(O)—” or “C₁-C₃ alkyl-NHC(O)—” refers to a linear or branched alkyl-NHC(O)— group containing from 1 to 3 carbon atoms. Representative examples of C₁₋₃ alkyl-NHC(O)— include, but are not limited to, CH₃—NHC(O)—, CH₃CH₂—NHC(O)—, and CH₃CH₂CH₂—NHC(O)—.

As used herein, the term “alkyl-C(O)NH—”, used alone or in combination, refers to a linear or branched alkyl-C(O)NH— group having structural formula of alkyl-C(O)NH—. Optionally, the alkyl portion of the alkyl-C(O)NH— group may contain 1-10 carbon atoms. The term “C₁₋₃ alkyl-C(O)NH—” or “C₁-C₃ alkyl-C(O)NH—” refers to a linear or branched alkyl-C(O)NH— group containing from 1 to 3 carbon atoms. Representative examples of C₁₋₃ alkyl-C(O)NH— include, but are not limited to, CH₃—C(O)NH—, CH₃CH₂—C(O)NH—, and CH₃CH₂CH₂—C(O)NH—.

As used herein, the term “heteroaryl”, used alone or in combination, refers to a 5- to 20-membered (e.g., 5- to 15-membered, 5- to 12-membered, 5- to 11-membered, 5- to 10-membered, 5 to 9-membered, 5- to 8-membered, 5- to 7-membered, 5- to 6-membered, 6- to 15-membered, or 6- to 9-membered) monocyclic, bicyclic, or polycyclic monovalent aromatic ring group containing one or more (e.g., from 1 to 6, or from 1 to 5, or from 1 to 4, or from 1 to 3) heteroatoms independently selected from the group consisting of oxygen, nitrogen, and sulfur. Bicyclic or polycyclic heteroaryl groups include bicyclic, tricyclic or tetracyclic heteroaryl groups, which contain one aromatic ring having one or more heteroatoms independently selected from O, S and N, and the remaining rings may be a saturated, partially unsaturated or aromatic ring and can be carbocyclic ring or contain one or more heteroatoms independently selected from O, S and N. Examples of monocyclic heteroaryl groups include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, tetrazolyl, and triazinyl. Examples of bicyclic heteroaryl groups include, but are not limited to, indolyl, isoindolyl, isoindolinyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, oxazolopyridyl, furopyridyl, pteridyl, purinyl, pyridopyridyl, pyrazolo[1,5-a]pyridyl, pyrazolo[1,5-a]pyrimidinyl, imidazo[1,2-a]pyridyl, 1H-pyrrolo[3,2-b]pyridyl, 1H-pyrrolo[2,3-b]pyridyl, pyrrolo[2,1-b]thiazolyl and imidazo[2,1-b]thiazolyl. Examples of tricyclic heteroaryl groups include, but are not limited to, acridinyl, benzindolyl, carbazolyl, dibenzofuranyl, and xanthyl. The heteroaryl group may be unsubstituted or substituted. A substituted heteroaryl refers to heteroaryl substituted one or more times (e.g., 1-4, 1-3, or 1-2 times) by a substituent(s) optionally selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “heteroarylene”, used alone or in combination, refers to a 5- to 20-membered (e.g., 5- to 15-membered, 5- to 12-membered, 5- to 11-membered, 5- to 10-membered, 5 to 9-membered, 5- to 8-membered, 5- to 7-membered, 5- to 6-membered, 6- to 15-membered, or 6- to 9-membered) monocyclic, bicyclic, or polycyclic bivalent aromatic ring group containing at least one aromatic ring having one or more (e.g., from 1 to 6, or from 1 to 5, or from 1 to 4, or from 1 to 3) heteroatoms independently selected from the group consisting of oxygen, nitrogen, and sulfur. Bicyclic or polycyclic heteroarylene groups include bicyclic, tricyclic or tetracyclic heteroarylene groups, which contain one aromatic ring having one or more heteroatoms independently selected from O, S and N, and the remaining ring(s) may be a saturated, partially unsaturated or aromatic ring and can be carbocyclic ring or contain one or more heteroatoms independently selected from O, S and N. Examples of monocyclic heteroarylene groups include, but are not limited to, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, tetrazolylene, and triazinylene. Examples of bicyclic heteroarylene groups include, but are not limited to, indolylene, isoindolylene, isoindolinylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, oxazolopyridylene, furopyridylene, pteridylene, purinylene, pyridopyridylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, pyrrolo[2,1-b]thiazolylene, and imidazo[2,1-b]thiazolylene. Examples of tricyclic heteroarylene groups include, but are not limited to, acridinylene, benzindolylene, carbazolylene, dibenzofuranylene, and xanthylene. The heteroarylene group may be unsubstituted or substituted. A substituted heteroarylene refers to heteroarylene substituted one or more times (e.g., 1-4, 1-3, or 1-2 times) by a substituent(s) optionally selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “aryl”, used alone or in combination, refers to a monovalent aromatic hydrocarbon group containing from 5 to 14 carbon atoms and optionally one or more fused rings, such as phenyl group, naphthyl group, or fluorenyl group. As used herein, the “aryl” is optionally substituted. A substituted aryl group refers to an aryl group substituted one or more times (e.g., 1-4, 1-3, or 1-2 times) with a substituent(s). For example, aryl is mono-, di-, or tri-substituted with a substituent(s) optionally selected from e.g., C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “arylene”, used alone or in combination, refers to a divalent aromatic hydrocarbon group containing from 5 to 14 carbon atoms and optionally one or more fused rings, such as phenylene, naphthylene, or fluorenylene. As used herein, the “arylene” is optionally substituted. A substituted arylene refers to an arylene group optionally substituted one or more times (e.g., 1-4, 1-3, or 1-2 times) with a substituent(s). For example, arylene is mono-, di-, or tri-substituted with a substituent(s) optionally selected from e.g., C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “cycloalkyl”, used alone or in combination, refers to a saturated or partially unsaturated (i.e., containing one or more double bonds, but not having a fully conjugated π-electron system) monocyclic or bicyclic or polycyclic cyclic hydrocarbon radical, which in some embodiments contains from 3 to 20 carbon atoms (i.e., C₃₋₂₀ cycloalkyl), or from 3 to 15 carbon atoms (i.e., C₃₋₁₅ cycloalkyl), or from 3 to 12 carbon atoms (i.e., C₃₋₁₂ cycloalkyl), or from 3 to 11 carbon atoms (i.e., C₃₋₁₁ cycloalkyl), or from 3 to 10 carbon atoms (i.e., C₃₋₁₀ cycloalkyl), or from 3 to 8 carbon atoms (i.e., C₃₋₈ cycloalkyl), or from 3 to 7 carbon atoms (i.e., C₃₋₇ cycloalkyl), or from 3 to 6 carbon atoms (i.e., C₃₋₆ cycloalkyl). The term “cycloalkyl” includes monocyclic, bicyclic, or tricyclic cyclic hydrocarbon radical having from 3 to 20 carbon atoms. Representative examples of monocyclic cycloalkyl groups include, but are not limited to, cyclopropyl, cyclobutyl, cyclopentyl, cyclopentenyl, cyclohexyl, cyclohexenyl, cycloheptyl, and cyclooctyl. Bicyclic and tricyclic cycloalkyl groups include bridged cycloalkyl, fused cycloalkyl and spiro-cycloalkyl groups such as, but not limited to, decalinyl, octahydropentalenyl, octahydro-1H-indenyl, spiro-cycloalkyl, adamantanyl, noradamantanyl, bornyl, norbornyl (also named as bicyclo[2.2.1]heptyl by the IUPAC system). As used herein, the “cycloalkyl” is optionally mono- or poly-substituted, such as, but not limited to, 2,2-, 2,3-, 2,4-, 2,5-, or 2,6-disubstituted cyclohexyl. The substituents of the substituted “cycloalkyl” can be optionally one or more (e.g., 1-5, 1-4, 1-3, 1-2, or 1) substituents selected from C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof. Examples of “C₃₋₆ cycloalkyl” include, but are not limited to, cyclopropyl, cyclobutyl, cyclopentyl, cyclopentenyl, and cyclohexyl.

As used herein, the term “cycloalkylene”, used alone or in combination, refers to a saturated or partially unsaturated (i.e., containing one or more double bonds, but not having a fully conjugated π-electron system) monocyclic or bicyclic or polycyclic divalent cyclic hydrocarbon radical containing from 3 to 12 carbon atoms (e.g., 3-12, 3-11, 3-10, 3-8, 3-7, 3-6 carbon atoms). The term “cycloalkylene” includes monocyclic, bicyclic or tricyclic cycloalkylene having from 3 to 12 carbon atoms. Representative examples of monocyclic cycloalkylene groups include, but are not limited to, cyclopropylene, cyclobutylene, cyclopentylene, cyclopentenylene, cyclohexylene, cyclohexenylene, cycloheptylene, and cyclooctylene. Bicyclic and tricyclic cycloalkylene groups include bridged cycloalkylene, fused cycloalkylene and spiro-cycloalkylene groups such as, but not limited to, decalinylene, octahydropentalenylene, octahydro-1H-indenylene, 2,3-dihydro-1H-indenylene, spiro-cycloalkylene, adamantanylene, noradamantanylene, and norbornylene (also named as bicyclo[2.2.1]heptylene by the IUPAC system). As used herein, the “cycloalkylene” is optionally mono- or poly-substituted, such as, but not limited to, 2,2-, 2,3-, 2,4-, 2,5-, or 2,6-disubstituted cyclohexylene. The substituents of the substituted “cycloalkylene” can be optionally one or more substituents selected from C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “C-y spiro-cycloalkylene” (x and y each being an integer), used alone or in combination, refers to a spiro-cycloalkylene group containing from x to y carbon atoms. As used herein, the term “C₇₋₁₁ spiro-cycloalkylene”, used alone or in combination, refers to a spiro-cycloalkylene group containing from 7 to 11 carbon atoms (e.g., 7-10, 7-9 carbon atoms). Representative examples of “C₇₋₁₁ spiro-cycloalkylene” include, but are not limited to, spiro[3.3]heptylene, spiro[2.5]octylene, spiro[3.5]nonylene, spiro[4.4]nonylene, spiro[4.5]decylene, or spiro[5.5]undecylene. The “C₇₋₁₁ spiro-cycloalkylene” is optionally further substituted with one or more substituents selected from the group consisting of C₁₋₃ alkyl, C₃₋₆ cycloalkyl, hydroxyl, amino, mercapto, halogen, C₁₋₃ alkoxy, C₁₋₃ alkylamino, halogenated C₁₋₃ alkyl, amino-substituted C₁₋₃ alkylene, C₁₋₃ alkyl-NHC(O)—, C₁₋₃ alkyl-C(O)NH—, cyano, or any combination thereof.

As used herein, the term “heterocyclyl” or “heterocyclic group”, used alone or in combination, refers to a 3- to 20-membered saturated or partially unsaturated (i.e., containing one or more double bonds, but not having a fully conjugated π-electron system) monocyclic, bicyclic, or tricyclic cyclic hydrocarbon group containing one or more (e.g., from 1 to 5, or from 1 to 4, from 1 to 3, from 1 to 2, or 1) heteroatoms independently selected from sulfur, oxygen, and nitrogen. In some embodiments, “heterocyclyl” may refer to a 3- to 15-membered (e.g., 3- to 14-membered, 3- to 12-membered, 3- to 11-membered, 3- to 10-membered, 3- to 9-membered, 3- to 8-membered, 3- to 7-membered, 3- to 6-membered, 3- to 5-membered, or 4- to 9-membered) saturated or partially unsaturated (i.e., containing one or more double bonds, but not having a fully conjugated π-electron system) monocyclic cyclic hydrocarbon group containing one or more (e.g., from 1 to 5, or from 1 to 4, from 1 to 3, from 1 to 2, or 1) heteroatoms independently selected from sulfur, oxygen, and nitrogen. Representative examples of the monocyclic heterocyclyl include, but are not limited to, azetidinyl, oxetanyl, pyrrolidinyl, imidazolidinyl, pyrazolidyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, dioxacyclohexyl, azacycloheptyl, azacyclooctyl, diazacycloheptanyl (e.g., 1,4-diazacycloheptan-1-yl), and diazacyclooctyl. Bicyclic and tricyclic heterocyclyl groups include bridged heterocyclyl, fused heterocyclyl and spiro-heterocyclyl groups such as, but not limited to, 6-azabicyclo[3.1.1]heptan-3-yl, 2,5-diazabicyclo[2.2.1]heptan-2-yl, 3,6-diazabicyclo[3.1.1]heptan-3-yl, 3-azabicyclo[3.2.1]octan-8-yl, 3,8-diazabicyclo[3.2.1]octan-8-yl, 3,8-diazabicyclo[3.2.1]octan-3-yl, 2,5-diazabicyclo[2.2.2]octan-2-yl, and azaspirocycloalkyl (including 3-azaspiro[5.5]undecan-3-yl). The heterocyclyl may be unsubstituted or substituted as explicitly defined (e.g., mono-, di-, tri-, or poly-substituted) by a substituent(s) optionally selected from the group consisting of deuterium, hydroxyl, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof.

As used herein, the term “nitrogen-containing monocyclic heterocyclyl”, used alone or in combination, refers to 3- to 20-membered (e.g., 3- to 15-membered, 3- to 14-membered, 3- to 12-membered, 3- to 11-membered, 3- to 10-membered, 3- to 9-membered, 3- to 8-membered, 3- to 7-membered, 3- to 6-membered, 3- to 5-membered, or 4- to 9-membered) saturated or partially unsaturated (i.e., containing one or more double bonds, but not having a fully conjugated π-electron system) monocyclic monovalent cyclic hydrocarbon group containing one nitrogen atom and optionally one or more (e.g., from 1 to 5, or from 1 to 4, from 1 to 3, from 1 to 2, or 1) heteroatoms independently selected from sulfur, oxygen, and nitrogen. Representative examples of the nitrogen-containing monocyclic heterocyclyl include, but are not limited to, azetidinyl, pyrrolidinyl, imidazolidinyl, pyrazolidyl, oxazolidinyl, thiazolidinyl, piperidinyl, piperazinyl, morpholinyl, thiomorpholinyl, azacycloheptyl, azacyclooctyl, diazacycloheptanyl (e.g., 1,4-diazacycloheptan-1-yl), and diazacyclooctyl. The nitrogen-containing monocyclic heterocyclyl may be unsubstituted or substituted as explicitly defined (e.g., mono-, di-, tri-, or poly-substituted) by a substituent(s) optionally selected from the group consisting of deuterium, hydroxyl, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof.

As used herein, the term “nitrogen-containing bridged heterocyclyl”, used alone or in combination, refers to 3- to 20-membered (e.g., 3- to 15-membered, 3- to 14-membered, 3- to 12-membered, 3- to 11-membered, 3- to 10-membered, 3- to 9-membered, 3- to 8-membered, 3- to 7-membered, 3- to 6-membered, 3- to 5-membered, or 4- to 9-membered) saturated or partially unsaturated (i.e., containing one or more double bonds, but not having a fully conjugated π-electron system) tricyclic monovalent cyclic hydrocarbon group containing one nitrogen atom and optionally one or more (e.g., from 1 to 5, or from 1 to 4, from 1 to 3, from 1 to 2, or 1) heteroatoms independently selected from sulfur, oxygen, and nitrogen. Tricyclic heterocyclyl groups include bridged heterocyclyl, such as but not limited to, 6-azabicyclo[3.1.1]heptan-3-yl, 2,5-diazabicyclo[2.2.1]heptan-2-yl, 3,6-diazabicyclo[3.1.1]heptan-3-yl, 3-azabicyclo[3.2.1]octan-8-yl, 3,8-diazabicyclo[3.2.1]octan-8-yl, 3,8-diazabicyclo[3.2.1]octan-3-yl, and 2,5-diazabicyclo[2.2.2]octan-2-yl. The nitrogen-containing bridged heterocyclyl may be unsubstituted or substituted as explicitly defined (e.g., mono-, di-, tri-, or poly-substituted) by a substituent(s) optionally selected from the group consisting of deuterium, hydroxyl, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof.

As used herein, the term “heterocyclylene”, used alone or in combination, refers to a 3- to 20-membered saturated or partially unsaturated (i.e., containing one or more double bonds, but not having a fully conjugated π-electron system) monocyclic, bicyclic, or tricyclic bivalent cyclic hydrocarbon group containing one or more (e.g., from 1 to 5, or from 1 to 4, from 1 to 3, from 1 to 2, or 1) heteroatoms independently selected from sulfur, oxygen, and nitrogen. In some embodiments, “heterocyclylene” may refer to e.g., a 3- to 15-membered (e.g., 3- to 14-membered, 3- to 12-membered, 3- to 11-membered, 3- to 10-membered, 3- to 9-membered, 3- to 8-membered, 3- to 7-membered, 3- to 6-membered, 3- to 5-membered, or 4- to 9-membered) saturated or partially unsaturated (i.e., containing one or more double bonds, but not having a fully conjugated π-electron system) monocyclic bivalent cyclic hydrocarbon group containing one or more (e.g., from 1 to 5, or from 1 to 4, from 1 to 3, from 1 to 2, or 1) heteroatoms independently selected from sulfur, oxygen, and nitrogen. Representative examples of the monocyclic heterocyclylene include, but are not limited to, azetidinylene, oxetanylene, pyrrolidinylene, imidazolidinylene, pyrazolidylene, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, dioxacyclohexylene, and diazacycloheptanylene (e.g., 1,4-diazacycloheptanylene, 4,5-diazacycloheptanylene, 1,3-diazacycloheptanylene). Bicyclic and tricyclic heterocyclylene groups include bridged heterocyclylene, fused heterocyclylene and spiro-heterocyclylene groups such as, but not limited to, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, 2,5-diazabicyclo[2.2.2]octanylene, and azaspirocycloalkylene (e.g., 3-azaspiro[5.5]undecanylene). The heterocyclylene may be unsubstituted or substituted as explicitly defined (e.g., mono-, di-, tri-, or poly-substituted) by a substituent(s) optionally selected from the group consisting of deuterium, hydroxyl, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof.

As used herein, the term “nitrogen-containing monocyclic heterocyclylene”, used alone or in combination, refers to 3- to 20-membered (e.g., 3- to 15-membered, 3- to 14-membered, 3- to 12-membered, 3- to 11-membered, 3- to 10-membered, 3- to 9-membered, 3- to 8-membered, 3- to 7-membered, 3- to 6-membered, 3- to 5-membered, or 4- to 9-membered) saturated or partially unsaturated (i.e., containing one or more double bonds, but not having a fully conjugated π-electron system) monocyclic bivalent cyclic hydrocarbon group containing one nitrogen atom and optionally one or more (e.g., from 1 to 5, or from 1 to 4, from 1 to 3, from 1 to 2, or 1) heteroatoms independently selected from sulfur, oxygen, and nitrogen. Representative examples of the nitrogen-containing monocyclic heterocyclylene include, but are not limited to, piperidinylene, piperazinylene, morpholinylene, azetidinylene, pyrrolidinylene, imidazolidinylene, pyrazolidylene, oxazolidinylene, thiazolidinylene, thiomorpholinylene, azacycloheptylene, diazacycloheptanylene, azacyclooctylene, and diazacyclooctylene. The nitrogen-containing monocyclic heterocyclylene may be unsubstituted or substituted as explicitly defined (e.g., mono-, di-, tri-, or poly-substituted) by a substituent(s) optionally selected from the group consisting of deuterium, hydroxyl, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof.

As used herein, the term “nitrogen-containing bridged heterocyclylene”, used alone or in combination, refers to 3- to 20-membered (e.g., 3- to 15-membered, 3- to 14-membered, 3- to 12-membered, 3- to 11-membered, 3- to 10-membered, 3- to 9-membered, 3- to 8-membered, 3- to 7-membered, 3- to 6-membered, 3- to 5-membered, or 4- to 9-membered) saturated or partially unsaturated (i.e., containing one or more double bonds, but not having a fully conjugated π-electron system) tricyclic bivalent cyclic hydrocarbon group containing one nitrogen atom and optionally one or more (e.g., from 1 to 5, or from 1 to 4, from 1 to 3, from 1 to 2, or 1) heteroatoms independently selected from sulfur, oxygen, and nitrogen. Tricyclic heterocyclylene groups include bridged heterocyclylene, such as but not limited to, 6-azabicyclo[3.1.1]heptanylene, 2,5-diazabicyclo[2.2.1]heptanylene, 3,6-diazabicyclo[3.1.1]heptanylene, 3-azabicyclo[3.2.1]octanylene, 3,8-diazabicyclo[3.2.1]octanylene, 3,8-diazabicyclo[3.2.1]octanylene, and 2,5-diazabicyclo[2.2.2]octanylene. The nitrogen-containing heterocyclylene may be unsubstituted or substituted as explicitly defined (e.g., mono-, di-, tri-, or poly-substituted) by a substituent(s) optionally selected from the group consisting of deuterium, hydroxyl, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof.

As used herein, the term “alkynylene”, used alone or in combination, refers to a linear or branched bivalent hydrocarbon group containing from 2 to 8 (e.g., from 2 to 6, from 2 to 5, from 2 to 4, or preferably 2) carbon atoms and having one or more (e.g., from 1 to 3, from 1 to 2, or 1) carbon-carbon triple bonds. Examples of alkynylene include, but are not limited to, ethynylene, 1-propynylene, 1-butynylene, and 1,3-diynylene.

As used herein, the term “alkynyl”, used alone or in combination, refers to a linear or branched monovalent hydrocarbon group containing from 2 to 8 (e.g., from 2 to 6, from 2 to 5, from 2 to 4, or preferably 2) carbon atoms and having one or more (e.g., from 1 to 3, from 1 to 2, or 1) carbon-carbon triple bonds. Examples of C₂₋₆ alkynylene include, but are not limited to, ethynyl, 1-propynyl, 1-butynyl, and 1,3-diynyl.

As used herein, the term “alkenylene”, used alone or in combination, refers to a linear or branched divalent hydrocarbon group containing from 2 to 8 (e.g., from 2 to 6, from 2 to 5, from 2 to 4, from 2 to 3, or 2) carbon atoms and having one or more (e.g., from 1 to 3, from 1 to 2, or 1) carbon-carbon double bonds. Examples of alkenylene groups include, but are not limited to, vinylene (e.g., —CH═CH—), 1-propenylene, allylidene, 1-butenylene, 2-butenylene, 3-butenylene, isobutenylene, pentenylene, n-pent-2,4-dienylene, 1-methyl-but-1-enylene, 2-methyl-but-1-enylene, 3-methyl-but-1-enylene, 1-methyl-but-2-enylene, 2-methyl-but-2-enylene, 3-methyl-but-2-enylene, 1-methyl-but-3-enylene, 2-methyl-but-3-enylene, 3-methyl-but-3-enylene, and hexenylene.

As used herein, the term “alkenyl”, used alone or in combination, refers to a linear or branched monovalent hydrocarbon group containing from 2 to 8 (e.g., from 2 to 6, from 2 to 5, from 2 to 4, from 2 to 3, or 2) carbon atoms and having one or more (e.g., from 1 to 3, from 1 to 2, or 1) carbon-carbon double bonds. Examples of C₂₋₆ alkenyl group include, but are not limited to, vinyl (e.g., CH₂═CH—), 1-propenyl, allyl, 1-butenyl, 2-butenyl, 3-butenyl, isobutenyl, pentenyl, n-pent-2,4-dienyl, 1-methyl-but-1-enyl, 2-methyl-but-1-enyl, 3-methyl-but-1-enyl, 1-methyl-but-2-enyl, 2-methyl-but-2-enyl, 3-methyl-but-2-enyl, 1-methyl-but-3-enyl, 2-methyl-but-3-enyl, 3-methyl-but-3-enyl, and hexenyl.

As used herein, “bornylane” or “bornane” (also known as 1,7,7-trimethylbicyclo[2.2.1]heptane; camphane; bornylane) has a definition known to those skilled in the art. As used herein, “camphanyl” or “bornyl” refers to a monovalent group of bornane, i.e., the group remaining after any one of the hydrogens in bornane is removed. Representative examples of “bornyl” include, but are not limited to, 1,7,7-trimethylbicyclo[2.2.1]heptan-2-yl, 1,7,7-trimethylbicyclo[2.2.1]heptan-3-yl, 1,7,7-trimethylbicyclo[2.2.1]heptan-4-yl, 1,7,7-trimethylbicyclo[2.2.1]heptan-5-yl, 1,7,7-trimethylbicyclo[2.2.1]heptan-6-yl,

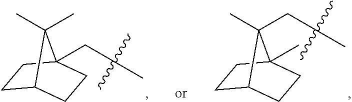 [SMILES: *CC12CCC(CC1)C2(C)C ; *CC1(C)C2CCC1(C)CC2]

As used herein, “bicyclo[2.2.1]heptane” also known as “norbornane”, has a definition known to those skilled in the art. As used herein, “bicyclo[2.2.1]heptyl” or “norbornyl” refers to a monovalent group of bicyclo[2.2.1]heptane, i.e., the group remaining after any hydrogen in bicyclo[2.2.1]heptane is removed. Representative examples of “bicyclo[2.2.1]heptyl” include, but are not limited to, bicyclo[2.2.1]heptan-2-yl, bicyclo[2.2.1]heptan-3-yl, bicyclo[2.2.1]heptan-4-yl, bicyclo[2.2.1]heptan-5-yl, or bicyclo[2.2.1]heptan-6-yl.

As used herein, the term “bicyclo[2.2.1]heptene” has a definition known to those skilled in the art. As used herein, “bicyclo[2.2.1]heptenyl” refers to a monovalent group of bicyclo[2.2.1]heptene, i.e., the group remaining after any hydrogen in bicyclo[2.2.1]heptene is removed. Representative examples of “bicyclo[2.2.1]heptenyl” include, but are not limited to, bicyclo[2.2.1]hept-5-en-2-yl, bicyclo[2.2.1]hept-5-en-3-yl, or bicyclo[2.2.1]hept-5-en-7-yl.

As used herein, “adamantane” (also known as tricyclo[3.3.1.1^{3,7}]decane) has a definition known to those skilled in the art, and its structural formula is e.g., as follows:

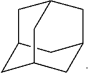

As used herein, “adamantanyl” refers to a monovalent group of adamantane, that is, the group remaining after any hydrogen in adamantane is removed. Representative examples of “adamantanyl” include, but are not limited to, 1-adamantanyl, 2-adamantanyl, 3-adamantanyl, 4-adamantanyl, 5-adamantanyl, 6-adamantanyl, 7-adamantanyl, 8-adamantanyl, 9-adamantanyl, or 10-adamantanyl.

As used herein, “noradamantane” has a definition known to those skilled in the art, and its structural formula is e.g., as follows:

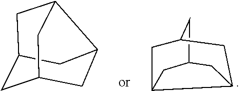

As used herein, “noradamantanyl” refers to a monovalent group of noradamantane, that is, the group remaining after any hydrogen in noradamantane is removed. Representative examples of “noradamantanyl” include, but are not limited to, 1-noradamantanyl, 2-noradamantanyl, 3-noradamantanyl, 4-noradamantanyl, 5-noradamantanyl, 6-noradamantanyl, 7-noradamantanyl, 8-noradamantanyl or 9-noradamantanyl.

As used herein, “adamantanamine” has the definitions known to those skilled in the art, namely referring to an adamantane having an amino substituent, wherein the amino substituent can replace a hydrogen on a carbon at any position in the adamantane. An example of “adamantanamine” can be adamantan-1-amine (corresponding English chemical name is adamantan-1-amine or Tricyclo[3.3.1.1^{3,7}]decan-1-amine; CAS No.: 768-94-5), with the following structural Formula:

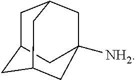 [SMILES: NC12CC3CC(CC(C3)C1)C2]

In the present disclosure, the term “leaving group” used alone or in combination is well known to those skilled in the art, which is a leaving molecular fragment (ion or neutral molecule) that carries a pair of electrons from a reactant in chemical reactions, as is a term used in nucleophilic substitution and elimination reactions. Common ionic leaving groups include Cl⁻, Br⁻, I⁻ and sulfonate (such as p-toluenesulfonate, TsO⁻), and neutral molecular leaving groups include water, ammonia and alcohol. In the present disclosure, those skilled in the art can select an appropriate leaving group as needed, such as but not limited to —N₃, halogen, methanesulfonyloxy, trifluoromethanesulfonyloxy or p-toluenesulfonyloxy, etc.

Salts or pharmaceutically acceptable salts, enantiomers, stereoisomers, solvates, polymorphs of the compounds of Formula (I), Formula (II) or Formula (III) of the present disclosure are also encompassed within the scope of the present invention.

In all embodiments of the present disclosure, the salts or pharmaceutically acceptable salts of the compounds of Formula (I), Formula (II) or Formula (III) refer to non-toxic inorganic or organic acid and/or base addition salts. Examples include: sulfates, hydrohalides (including hydrochlorides and hydrobromates), maleates, sulfonates, citrates, lactates, lactobionates, L-tartrates, fumarates, L-malates, L-lactates, α-Ketoglutarates, hippurates, D-glucuronates, D-gluconates, α-D-glucoheptonates, glycolates, mucates, L-ascorbates, orotates, picrates, glycinates, alaninates, argininates, cinnamates, laurates, pamoates, sebacates, benzenesulfonates, methanesulfonates, ethanesulfonates, edisylates, formates, acetates, 2,2-dichloroacetates, trimethylacetates, propionates, valerates, palmitates, triphenylacetates, 2-ethylsuccinates, iodates, niacinates, L-pyroglutamates, L-prolinates, ferulates, 2-hydroxyethanesulfonates, nitrates, gentisates, cholates, salicylates, terephthalates, glutarates, adipates, stearates, oleates, undecenoates, camphorates, camphorsulfonates, dodecyl sulfonates, phosphates, thiocyanates, dihydrophosphates, pyrophosphates, metaphosphates, oxalates, carbonates, malonates, benzoates, mandelates, succinates, pyruvates, para-chlorobenzenesulfonates, 1,5-naphthalenedisulfonates, 3-hydroxy-2-naphthoates, 1-hydroxy-2-naphthoates, 2-naphthalenesulfonates, hydroxyacetates, or p-toluenesulfonates, etc.

“Pharmaceutically acceptable carrier” refers to a pharmaceutically acceptable material, such as a filler, stabilizer, dispersant, suspending agent, diluent, excipient, thickener, solvent, or encapsulating material, with which the useful compounds according to the present disclosure are carried or transported into or administered to a patient so that they can perform their intended function. Generally, such constructs are carried or transported from one organ or part of the body to another organ or part of the body. The carrier is compatible with the other ingredients of the formulation, including the compounds useful in the present disclosure, and is not harmful to the patient, and the carrier must be “acceptable”. Some examples of materials that can be used as pharmaceutically acceptable carriers include sugars such as lactose, glucose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository wax; oils such as peanut oil, cottonseed oil, safflower oil, sesame oil, olive oil, corn oil and soybean oil; glycols such as propylene glycol; polyols such as glycerol, sorbitol, mannitol, and polyethylene glycol; esters such as ethyl oleate and ethyl laurate; agar; buffers such as magnesium hydroxide and aluminum hydroxide; surfactant phosphate buffer solution; and other common non-toxic compatible substances used in pharmaceutical formulations.

As used herein, the term “room temperature” refers to the ambient temperature, such as 20-30° C.

As used herein, “stereoisomer” refers to a compound with the same chemical structural formula, but a different arrangement of atoms or groups in space. Stereoisomers include enantiomers, diastereomers, conformational isomers (rotational isomers), geometric isomers (cis/trans isomers), atropisomers, and so on.

As used herein, the term “solvate” refers to an association or complex formed by the interaction between one or more solvent molecules and compounds of the present invention. Examples of solvents include water, isopropanol, ethanol, methanol, DMSO, ethyl acetate, acetic acid and ethanolamine. The term “hydrate” means that a complex formed with water.

As used herein, the term “chiral” refers to a molecule that is nonsuperimposable on its mirror image; whereas “achiral” refers to a molecule that can be superimposed on its mirror image.

As used herein, the term “enantiomers” refers to two isomers of a compound that are nonsuperimposable mirror images.

As used herein, the term “diastereomers” refers to stereoisomers that have two or more chiral centers but are not mirror images of each other. The diastereomers have different physical properties, such as melting point, boiling point, spectral properties and reactivity. The diastereomeric mixture can be separated by high-resolution analytical operations such as electrophoresis and chromatography, such as HPLC.

### EXAMPLES

In the following description, numerous specific details are set forth in order to provide a thorough understanding of the present disclosure. The present disclosure may be practiced without some or all of these specific details. In other cases, well-known process operations have not been described in detail in order not to unnecessarily obscure the present disclosure. Although the present disclosure will be described in conjunction with specific embodiments, it should be understood that this is not intended to limit the present disclosure to these embodiments.

The following abbreviations are used throughout the specification and examples:
AcOH acetic acid
Boc t-Butyloxy carbonyl
Con. concentration
DCM dichloromethane
DIEA or DIPEA N, N-diisopropylethylamine
DMF N,N-dimethylformamide
DMSO dimethyl sulfoxide
DIPEA N, N-diisopropylethylamine
DESS-MARTIN Dess-Martin oxidant
EA ethyl acetate
EDCI 1-(3-dimethylaminopropyl)-3-ethylcarbodiimide hydrochloride
ESI electrospray ionization
equiv equivalent
EtOH ethanol
HATU 2-(7-Azabenzotriazol-1-yl)-N,N,N′,N′-tetramethyluronium hexafluorophosphate
HPLC high performance liquid chromatography
HRMS high resolution mass spectrometry
LC-MS liquid chromatography-mass spectrometry
LRMS low resolution mass spectrometry
LC liquid chromatography
Me methyl
MeCN acetonitrile
MeOH Methanol
MS mass spectrometry
¹H NMR Proton nuclear magnetic resonance
rt room temperature
tBu tert-butyl
TEA triethylamine
TFA trifluoroacetic acid
TLC thin layer chromatography
TMS tetramethylsilane

In the present disclosure, the ¹H NMR spectrum was recorded on a Bruker-500 MHz nuclear magnetic resonance instrument, by using, as a solvent, CD₃OD (δ=3.31 ppm) containing 0.1% TMS (as an internal standard); or using, as a solvent, CDCl₃ (δ=7.26 ppm) containing 0.1% TMS (as an internal standard); or using, as a solvent, DMSO-d₆ (δ=2.50 ppm) containing 0.03% TMS (as an internal standard). LRMS spectrum was recorded on an AB Triple 4600 mass spectrometer, HPLC preparation was measured on a SHIMADZU LC-20AP type instrument, and HPLC purity was measured on a SHIMADZU LC-30AP or Waters 1525 type instrument. Unless otherwise specified, all reactions were performed in the air atmosphere. The reactions were monitored via TLC or LC-MS.

Solvents and reagents are processed as follows:
the solvents used in the reactions such as DCM, DMF, anhydrous EtOH, and anhydrous MeOH were commercially available, e.g., from Chinese Sinopharm Group;
HPLC preparation was performed by using preparative grade CH₃CN and deionized water;
unless otherwise specified, various derivatives of PBM, various carbon chain linkers of different lengths (i.e., compounds used to form the groups of LIN), as well as other reagents and drugs were commercially available.

Unless otherwise specified, the materials and reagents used in the following examples are commercially available and used directly, or can be synthesized by using methods known in the art.

### General Synthesis Methods

Compounds and/or pharmaceutically acceptable salts thereof of the present disclosure can be synthesized using commercially available raw materials by synthetic techniques known in the art. The synthetic schemes described below illustrate the preparation of most compounds. The starting materials or reagents used in each scheme can be purchased from commercial sources or prepared by methods known to those skilled in the art. One skilled in the art can prepare the salts, racemates, enantiomers, phosphates, sulfates, hydrochlorides and prodrug forms of the compounds of Formula (I), Formula (II) and Formula (III) of the present disclosure according to routine techniques in the art.

The first general synthesis method of compounds of Formula (I):

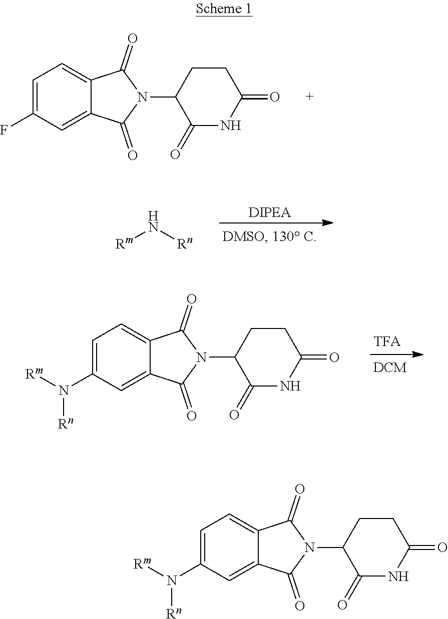

In Scheme 1, R^{m} of the substrate amine R^{m}—NH—Rⁿ represents hydrogen, and Rⁿ represents a nitrogen-containing heterocyclyl. Alternatively, R^{m} and Rⁿ, together with the nitrogen atom to which they are connected, form 4- to 10-membered monocyclic or bridged heterocyclyl containing 1-2 nitrogen atoms. One of the two nitrogen atoms in Rⁿ—NH—Rⁿ is protected by Boc.

The amine alkylation reaction in Step 1 of Scheme 1 can be carried out under conventional conditions well-known to those skilled in the art. For example, the amine alkylation reaction can also be performed at room temperature to 80° C. in the presence of DIEA and sodium iodide, or triethylamine and sodium iodide.

For example, Step 1: to a solution of 2-(2,6-dioxopiperidin-3-yl)-5-fluoroisoindoline-1,3-dione in DMSO (4 mL) were added the corresponding amine (1.1 equiv) and N,N-diisopropylethylamine (3 equiv). The reaction mixture was reacted at 130° C. for 3 h. After monitoring the reaction and confirming its completion, the reaction mixture was extracted using ethyl acetate. The organic phases were combined, washed with water, and dried over anhydrous sodium sulfate, and filtered. The filtrate was concentrated under reduced pressure, and the resulting residue was subjected to a column chromatography (DCM:EtOAc=2:1 to 1:1) for separation to give the Boc intermediate compound.

Step 2: to a solution of the product from step 1 in DCM (5 mL) was added CF₃CO₂H (1 mL). The reaction mixture was reacted at room temperature for 3 h, and concentrated under reduced pressure to give trifluoroacetate salt of the target compound.

The second general synthesis method of compounds of Formula (I):

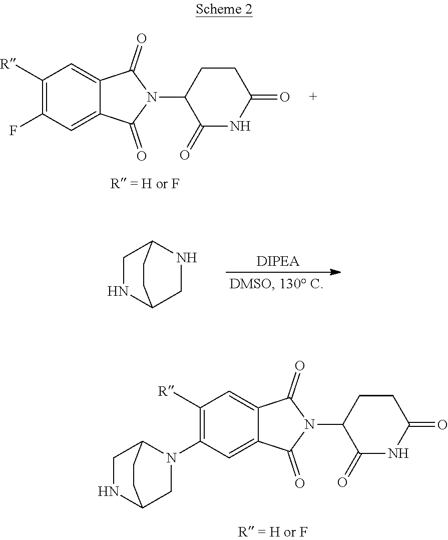

To a solution of the corresponding diketone (1.0 equiv) in DMSO (2 mL) were added 2,5-diazabicyclo[2.2.2]octane (2.0 equiv) and N,N-diisopropylethylamine (3 equiv). The reaction mixture was reacted at 130° C. for 3 h. After monitoring the reaction and confirming its completion, the reaction mixture was subjected to a C18 reversed-phase column chromatography (eluent (v/v): acetonitrile/(water+0.05% HCl)=10%-100%) for separation. The collected fractions were rotary evaporated to remove acetonitrile, and the resulting residue was lyophilized to afford the hydrochloride of the target compound.

The amine alkylation reaction in Scheme 2 can be carried out under conventional conditions well-known to those skilled in the art. For example, the amine alkylation reaction can also be performed at room temperature to 80° C. in the presence of DIEA and sodium iodide, or triethylamine and sodium iodide.

The first general synthesis method of compounds of Formula (II):

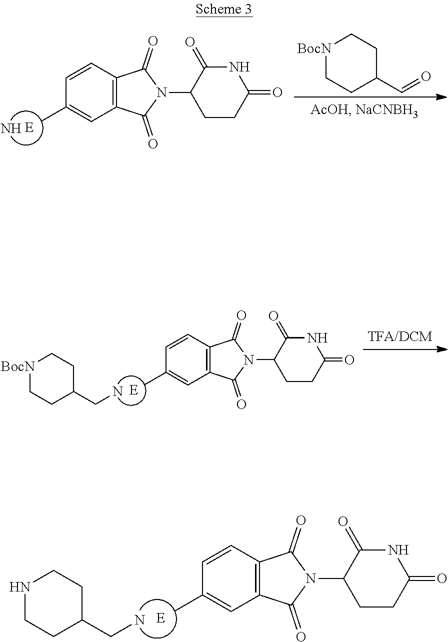

In Scheme 3, ring E represents the nitrogen-containing heterocyclyl as defined herein.

The reduction amination reaction in step 1 and the deprotection in step 2 of Scheme 3 can be conventional techniques and methods well-known to those skilled in the art. For example, the reduction amination reaction can be carried out in the presence of sodium triacetoxyborohydride and 1,2-dichloroethane at room temperature to 80° C. The deprotection can be carried out under the conditions of HCl/1,4-dioxane.

For example, Step 1: to a solution of tert-butyl 4-formylpiperidine-1-carboxylate (1.0 equiv) and amine (1.0 equiv) in dichloromethane was added an appropriate amount of acetic acid. The reaction mixture was allowed to react at room temperature for 30 minutes, followed by addition of sodium cyanoborohydride (3.0 equiv) and continued reaction at room temperature. After monitoring the reaction via LCMS and confirming its completion, the reaction mixture was subjected to a C18 reversed-phase column chromatography (eluent (v/v): acetonitrile/(water+0.05% HCl)=10%-100%) for separation. The collected fractions were rotary evaporated to remove acetonitrile, and the resulting residue was lyophilized to give the Boc intermediate compound.

Step 2: to a solution of the product from step 1 in DCM (5 mL) was added CF₃CO₂H (1 mL). The reaction mixture was reacted at room temperature for 3 h, and concentrated under reduced pressure to give trifluoroacetate of the target compound.

General synthesis method of compounds of Formula (III):

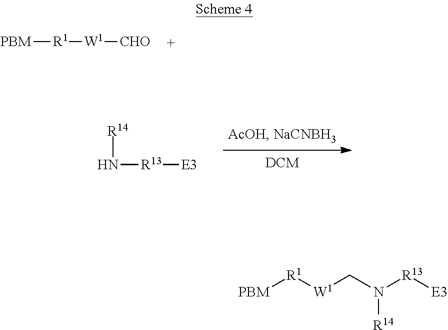

In Scheme 4, PBM, R₁, and W₁ are as defined in the compounds of Formula (III) of the present disclosure. The substrate R¹⁴—NH—R¹³-E3 is a form of the compounds of Formula (I), wherein E3 represents, for example, the domides core moiety in the compounds of Formula (I); the group R¹⁴ represents hydrogen, and R¹³ represents a nitrogen-containing heterocyclyl. Alternatively, R¹⁴ and R¹³ together with the nitrogen atom to which they are connected, form 4- to 10-membered monocyclic or bridged heterocyclyl containing 1-2 nitrogen atoms.

The reduction amination reaction in Scheme 4 can be conventional techniques and methods well-known to those skilled in the art. For example, the reduction amination reaction can be carried out in the presence of sodium triacetoxyborohydride and 1,2-dichloroethane at room temperature to 80° C.

For example, to a solution of the corresponding aldehyde (1.0 equiv) and amine (1.0 equiv) in dichloromethane was added an appropriate amount of acetic acid. The reaction mixture was allowed to react at room temperature for 30 minutes, followed by addition of sodium cyanoborohydride (3.0 equiv) and continued reaction at room temperature. After monitoring the reaction via LCMS and confirming its completion, the reaction mixture was subjected to a C18 reversed-phase column chromatography for separation. The collected fractions were rotary evaporated to remove acetonitrile, and the resulting residue was lyophilized to give the target compound.

The third general synthesis method of compounds of Formula (I):

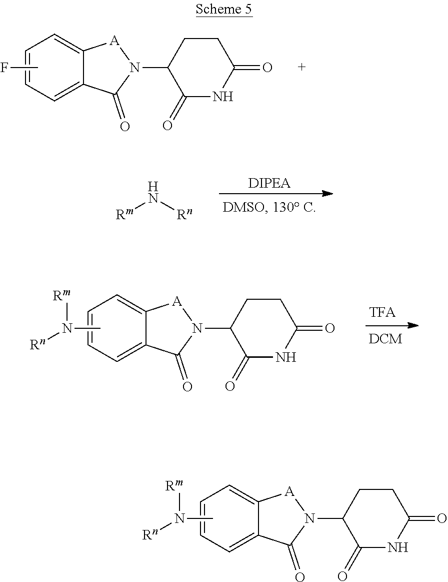

In Scheme 5, A represents C(O) or CH₂. R^{m} of the substrate amine R^{m}—NH—Rⁿ represents hydrogen, and Rⁿ represents a nitrogen-containing heterocyclyl. Alternatively, R^{m} and Rⁿ, together with the nitrogen atom to which they are connected, form 4- to 10-membered monocyclic or bridged heterocyclyl containing 1-2 nitrogen atoms. One of the two nitrogen atoms in R^{m}—NH—Rⁿ is protected by Boc.

The amine alkylation reaction in Step 1 of Scheme 5 can be carried out under conventional conditions well-known to those skilled in the art. For example, the amine alkylation reaction can also be performed at room temperature to 80° C. in the presence of DIEA and sodium iodide, or triethylamine and sodium iodide.

The fourth general synthesis method of compounds of Formula (I):

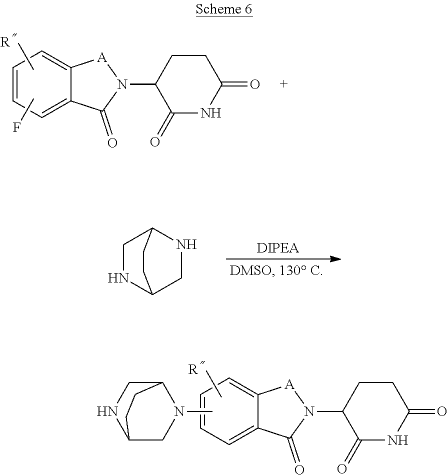

In Scheme 6, A represents C(O) or CH₂, R″ represents H or F.

The amine alkylation reaction in Scheme 6 can be carried out under conventional conditions well-known to those skilled in the art. For example, the amine alkylation reaction can also be performed at room temperature to 80° C. in the presence of DIEA and sodium iodide, or triethylamine and sodium iodide.

The second general synthesis method of compounds of Formula (II):

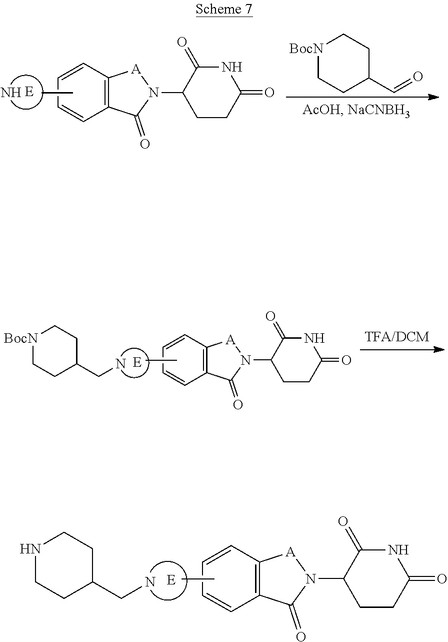

In Scheme 7, A represents C(O) or CH₂, ring E represents nitrogen-containing heterocyclyl as defined herein.

The reduction amination reaction in step 1 and the deprotection in step 2 of Scheme 7 can be conventional techniques and methods well-known to those skilled in the art. For example, the reduction amination reaction can be carried out in the presence of sodium triacetoxyborohydride and 1,2-dichloroethane at room temperature to 80° C. The deprotection can be carried out under the conditions of HCl/1,4-dioxane.

Depending upon the target compounds, the above schemes and their reaction substrates, reaction conditions (including reaction dosage, temperature, duration, etc.), work up, etc. can be appropriately modified and adjusted by techniques and methods well known to those skilled in the art to obtain the desired target compounds. The obtained target compounds can be further modified by the substituents and the like to obtain subsequent target compounds through methods well known to those skilled in the art.

### EXAMPLES

### Intermediate Example 1: Preparation of 5-(3,5-dimethylpiperazin-1-yl)-2-(2,6-dioxopiperidin-3-yl)isoindoline-1,3-dione (GT-03099)

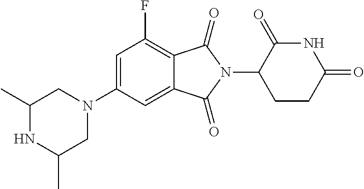 [SMILES: CC1CN(c2cc(F)c3c(c2)C(=O)N(C2CCC(=O)NC2=O)C3=O)CC(C)N1]

Referring to Scheme 1, 2-(2,6-dioxopiperidin-3-yl)-5-fluoroisoindoline-1,3-dione and tert-butyl 2,6-dimethylpiperazine-1-carboxylate were used to prepare the trifluoroacetate salt of the target compound (GT-03099) (yellow solid, 800.4 mg, yield 92%). ¹H NMR (400 MHz, DMSO-d₆) δ 11.08 (s, 1H), 9.31 (s, 1H), 8.67 (s, 1H), 7.74 (d, J=8.0 Hz, 1H), 7.51 (s, 1H), 7.37 (d, J=8.0 Hz, 1H), 5.09 (dd, J=12.0, 4.0 Hz, 1H), 4.25 (d, J=16.0 Hz, 2H), 3.47-3.38 (m, 4H), 2.88 (q, J=12.0 Hz, 2H), 2.62-2.50 (m, 2H), 1.31-1.01 (m, 6H). LCMS (ESI) calcd for C₁₉H₂₃N₄O₄⁺ [M+H]⁺, 371.42; found, 371.2.

### Intermediate Example 2: Preparation of 5-((R)-3-aminopiperidin-1-yl)-2-(2,6-dioxopiperidin-3-yl)isoindoline-1,3-dione (GT-03204)

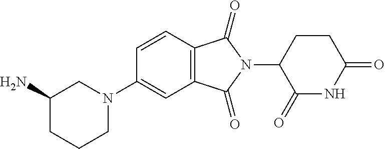 [SMILES: N[C@@H]1CCCN(c2ccc3c(c2)C(=O)N(C2CCC(=O)NC2=O)C3=O)C1]

Referring to Scheme 1, 2-(2,6-dioxopiperidin-3-yl)-5-fluoroisoindoline-1,3-dione and tert-butyl (R)-piperidin-3-ylcarbamate were used to prepare the trifluoroacetate salt of the target compound (GT-03204) (yellow solid, 1.1 g, 94%). LCMS (ESI) calcd for C₁₈H₂₁N₄O₄⁺ [M+H]⁺, 357.39; found, 357.2.

### Intermediate Example 3: Preparation of 5-((S)-3-aminopiperidin-1-yl)-2-(2,6-dioxopiperidin-3-yl)isoindoline-1,3-dione (GT-03200)

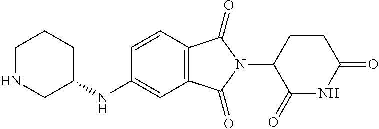 [SMILES: O=C1CCC(N2C(=O)c3ccc(N[C@H]4CCCNC4)cc3C2=O)C(=O)N1]

Referring to Scheme 1, 2-(2,6-dioxopiperidin-3-yl)-5-fluoroisoindoline-1,3-dione and tert-butyl (S)-3-aminopiperidin-1-carboxylate were used to prepare the trifluoroacetate salt of the target compound (GT-03202) (yellow solid, 518 mg, 55%). LCMS (ESI) calcd for C₁₈H₂₁N₄O₄⁺ [M+H]⁺, 357.39; found, 357.2.

### Intermediate Example 4: Preparation of 2-(2,6-dioxopiperidin-3-yl)-5-(((R)-piperidin-3-yl)amino)isoindoline-1,3-dione (GT-03203)

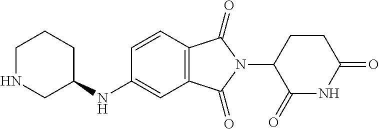 [SMILES: O=C1CCC(N2C(=O)c3ccc(N[C@@H]4CCCNC4)cc3C2=O)C(=O)N1]

Referring to Scheme 1, 2-(2,6-dioxopiperidin-3-yl)-5-fluoroisoindoline-1,3-dione and tert-butyl (R)-3-aminopiperidin-1-carboxylate were used to prepare the trifluoroacetate salt of the target compound (GT-03203) (yellow solid, 536 mg, 57%). LCMS (ESI) calcd for C₁₈H₂₁N₄O₄⁺ [M+H]⁺, 357.39; found, 357.2.

### Example 1: Preparation of 5-(3,6-diazabicyclo[3.1.1]heptan-6-yl)-2-(2,6-dioxopiperidin-3-yl)isoindoline-1,3-dione (GT-03100)

Referring to Scheme 1, 2-(2,6-dioxopiperidin-3-yl)-5-fluoroisoindoline-1,3-dione and tert-butyl 3,6-diazabicyclo[3.1.1]heptan-3-carboxylate were used to prepare the trifluoroacetate salt of the target compound (GT-03100) (yellow solid, 735.6 mg, yield 72%). ¹H NMR (400 MHz, DMSO-d₆) δ 11.08 (s, 1H), 9.57 (s, 1H), 8.70 (s, 1H), 7.76 (d, J=8.0 Hz, 1H), 7.18 (d, J=4.0 Hz, 1H), 6.97 (dd, J=8.0, 2.4 Hz, 1H), 5.09 (dd, J=12.0, 4.0 Hz, 1H), 4.61 (d, J=4.0 Hz, 2H), 3.58-3.53 (m, 2H), 3.31 (d, J=12.0 Hz, 2H), 2.92-2.83 (m, 2H), 2.63-2.49 (m, 2H), 2.05-1.96 (m, 2H). LCMS (ESI) calcd for C₁₈H₁₉N₄O₄⁺ [M+H]⁺, 355.37; found, 355.10.

### Example 2: Preparation of 5-(3,6-diazabicyclo[3.1.1]heptan-3-yl)-2-(2,6-dioxopiperidin-3-yl)isoindoline-1,3-dione (GT-03075)

Referring to Scheme 1, 2-(2,6-dioxopiperidin-3-yl)-5-fluoroisoindoline-1,3-dione and tert-butyl 3,6-diazabicyclo[3.1.1]heptan-6-carboxylate were used to prepare the trifluoroacetate salt of the target compound (GT-03075) (yellow solid, 1.23 g, yield 80%). ¹H NMR (400 MHz, DMSO-d₆) δ 11.09 (s, 1H), 9.76 (s, 1H), 8.36 (s, 1H), 7.79 (d, J=12 Hz, 1H), 7.20-7.11 (m, 2H), 5.09 (dd, J=13.0, 5.4 Hz, 1H), 4.53 (d, J=4.0 Hz, 2H), 4.06-3.96 (m, 2H), 3.34-3.32 (m, 2H), 2.94-2.85 (m, 2H), 2.63-2.51 (m, 2H), 2.07-1.99 (m, 2H). LCMS (ESI) calcd for C₁₈H₁₉N₄O₄⁺ [M+H]⁺, 355.37; found, 355.10.

### Example 3: Preparation of 5-(3,8-diazabicyclo[3.2.1]octan-8-yl)-2-(2,6-dioxopiperidin-3-yl)isoindoline-1,3-dione (GT-03210)

Referring to Scheme 1, 2-(2,6-dioxopiperidin-3-yl)-5-fluoroisoindoline-1,3-dione and tert-butyl 3,8-diazabicyclo[3.2.1]octan-3-carboxylate were used to prepare the trifluoroacetate salt of the target compound (GT-03210) (yellow solid, 820.8 mg, yield 83%). ¹H NMR (400 MHz, DMSO-d₆) δ 11.08 (s, 1H), 9.24 (s, 1H), 9.08 (s, 1H), 7.73 (d, J=12.0 Hz, 1H), 7.38 (s, 1H), 7.25 (d, J=12.0 Hz, 1H), 5.08 (dd, J=12.0, 4.0 Hz, 1H), 4.71 (s, 2H), 3.10 (d, J=4.0 Hz, 4H), 2.93-2.84 (m, 1H), 2.62-2.50 (m, 1H), 2.16-2.00 m, 6H). LCMS (ESI) calcd for C₁₉H₂₁N₄O₄⁺ [M+H]⁺, 369.40; found, 369.20.

### Example 4: Preparation of 5-(3,8-diazabicyclo[3.2.1]octan-3-yl)-2-(2,6-dioxopiperidin-3-yl)isoindoline-1,3-dione (GT-03077)

Referring to Scheme 1, 2-(2,6-dioxopiperidin-3-yl)-5-fluoroisoindoline-1,3-dione and tert-butyl 3,8-diazabicyclo[3.2.1]octan-8-carboxylate were used to prepare the trifluoroacetate salt of the target compound (GT-03077) (yellow solid, 672.1 mg, yield 86%). ¹H NMR (400 MHz, DMSO-d₆) δ 11.09 (s, 1H), 9.17 (s, 2H), 7.74 (d, J=8.0 Hz, 1H), 7.38 (s, 1H), 7.25 (d, J=8.0 Hz, 1H), 5.08 (d, J=8.0 Hz, 1H), 4.19 (s, 2H), 3.97 (d, J=12.0 Hz, 2H), 3.24 (d, J=12.0 Hz, 2H), 2.89 (t, J=12.0 Hz, 1H), 2.62-2.51 (m, 3H), 2.04-1.92 (m, 4H). LCMS (ESI) calcd for C₁₉H₂₁N₄O₄⁺ [M+H]⁺, 369.40; found, 369.20.

### Example 5: Preparation of 5-(2,5-diazabicyclo[2.2.2]octan-2-yl)-2-(2,6-dioxopiperidin-3-yl)isoindoline-1,3-dione (GT-03076)

Referring to Scheme 2, 2-(2,6-dioxopiperidin-3-yl)-5-fluoroisoindoline-1,3-dione and 2,5-diazabicyclo[2.2.2]octane were used to prepare the hydrochloride salt of the target compound (GT-03076) (brown solid, 173 mg, 29%). ¹H NMR (400 MHz, DMSO-d₆) δ 11.06 (s, 1H), 9.45 (s, 2H), 7.70 (d, J=8.0 Hz, 1H), 7.17 (s, 1H), 7.01 (d, J=8.0 Hz, 1H), 5.06 (dd, J=12.0, 4.0 Hz, 1H), 4.50 (s, 2H), 3.87-3.73 (m, 2H), 3.57-3.52 (m, 2H), 2.88-2.85 (m, 1H), 2.61-2.50 (m, 3H), 2.15-1.83 (m, 4H). LCMS (ESI) calcd for C₁₉H₂₁N₄O₄⁺ [M+H]⁺, 369.40; found, 369.30.

### Example 6: Preparation of 5-(3,6-diazabicyclo[3.1.1]heptan-6-yl)-2-(2,6-dioxopiperidin-3-yl)-6-fluoroisoindoline-1,3-dione (GT-03106)

Referring to Scheme 1, 2-(2,6-dioxopiperidin-3-yl)-5,6-difluoroisoindoline-1,3-dione and tert-butyl 3,6-diazabicyclo[3.1.1]heptane-3-carboxylate were used to prepare the trifluoroacetate salt of the target compound (GT-03106) (yellow solid, 573.4 mg, yield 87%). ¹H NMR (400 MHz, DMSO-d₆) δ 11.11 (s, 1H), 9.34 (s, 1H), 8.55 (s, 1H), 7.79 (d, J=12.0 Hz, 1H), 7.39 (d, J=8.0 Hz, 1H), 5.11 (dd, J=12.0, 4.0 Hz, 1H), 4.63-4.61 (m, 2H), 3.47-3.36 (m, 4H), 2.96-2.85 (m, 2H), 2.67-2.49 (m, 2H), 2.06-1.96 (m, 2H). LCMS (ESI) calcd for C₁₈H₁₈FN₄O₄⁺ [M+H]⁺, 373.36; found, 373.1.

### Example 7: Preparation of 5-(3,6-diazabicyclo[3.1.1]heptan-3-yl)-2-(2,6-dioxopiperidin-3-yl)-6-fluoroisoindoline-1,3-dione (GT-03109)

Referring to Scheme 1, 2-(2,6-dioxopiperidin-3-yl)-5,6-difluoroisoindoline-1,3-dione and tert-butyl 3,6-diazabicyclo[3.1.1]heptane-6-carboxylate were used to prepare the trifluoroacetate salt of the target compound (GT-03109) (yellow solid, 847.1 mg, yield 69%). LCMS (ESI) calcd for C₁₈H₁₈FN₄O₄⁺ [M+H]⁺, 373.36; found, 373.1.

### Example 8: Preparation of 5-(3,8-diazabicyclo[3.2.1]octan-8-yl)-2-(2,6-dioxopiperidin-3-yl)-6-fluoroisoindoline-1,3-dione (GT-03211)

Referring to Scheme 1, 2-(2,6-dioxopiperidin-3-yl)-5,6-difluoroisoindoline-1,3-dion and tert-butyl 3,8-diazabicyclo[3.2.1]octane-3-carboxylate were used to prepare the trifluoroacetate salt of the target compound (GT-03211) (1.65 mg, yield 92%). ¹H NMR (400 MHz, DMSO-d₆) δ 11.12 (s, 1H), 9.09 (s, 1H), 8.85 (s, 1H), 7.78 (d, J=12.0 Hz, 1H), 7.57 (d, J=8.0 Hz, 1H), 5.11 (dd, J=12.0, 8.0 Hz, 1H), 4.63 (t, J=4.0 Hz, 2H), 3.23-3.15 (m, 4H), 2.94-2.85 (m, 1H), 2.68-2.51 (m, 3H), 2.15-2.03 (m, 4H). LCMS (ESI) calcd for C₁₉H₂₀FN₄O₄⁺ [M+H]⁺, 387.39; found, 387.1.

### Example 9: Preparation of 5-(3,8-diazabicyclo[3.2.1]octan-3-yl)-2-(2,6-dioxopiperidin-3-yl)-6-fluoroisoindoline-1,3-dione (GT-03107)

Referring to Scheme 1, 2-(2,6-dioxopiperidin-3-yl)-5,6-difluoroisoindoline-1,3-dion and tert-butyl 3,8-diazabicyclo[3.2.1]octane-8-carboxylate were used to prepare the trifluoroacetate salt of the target compound (GT-03107) (yellow solid, 1.27 g, yield 86%). ¹H NMR (400 MHz, DMSO-d₆) δ 11.10 (s, 1H), 9.04-8.98 (m, 2H), 7.79 (d, J=12.0 Hz, 1H), 7.51 (d, J=4.0 Hz, 1H), 5.11 (dd, J=16.0, 8.0 Hz, 1H), 4.15 (s, 2H), 3.58-3.55 (m, 2H), 3.34 (d, J=12.0 Hz, 2H), 2.93-2.84 (m, 1H), 2.63-2.51 (m, 3H), 2.06-1.98 (m, 4H). LCMS (ESI) calcd for C₁₉H₂₀FN₄O₄⁺ [M+H]⁺, 387.39; found, 387.1.

### Example 10: Preparation of 5-((1S,4S)-2,5-diazabicyclo[2.2.1]heptan-2-yl)-2-(2,6-dioxopiperidin-3-yl)-6-fluoroisoindoline-1,3-dione (GT-03103)

Referring to Scheme 1, 2-(2,6-dioxopiperidin-3-yl)-5,6-difluoroisoindoline-1,3-dion and tert-butyl (1S,4S)-2,5-diazabicyclo[2.2.1]heptan-2-carboxylate were used to prepare the trifluoroacetate salt of the target compound (GT-03103) (yellow solid, 1.89 g, yield 88%). ¹H NMR (400 MHz, DMSO-d₆) δ 11.09 (s, 1H), 9.06 (s, 1H), 8.57 (s, 1H), 7.72 (d, J=12.0 Hz, 1H), 7.32 (d, J=8.0 Hz, 1H), 5.08 (dd, J=12.0, 4.0 Hz, 1H), 4.88 (s, 1H), 4.47 (s, 1H), 3.89 (d, J=12.0 Hz, 1H), 2.93-2.84 (m, 1H), 2.67-2.51 (m, 4H), 2.18 (d, J=8.0 Hz, 1H), 2.07-1.94 (m, 3H). LCMS (ESI) calcd for C₁₈H₁₈FN₄O₄⁺ [M+H]⁺, 373.36; found, 373.1.

### Example 11: Preparation of 5-(3,5-dimethylpiperazin-1-yl)-2-(2,6-dioxopiperidin-3-yl)-6-fluoroisoindoline-1,3-dione (GT-03104)

Referring to Scheme 1, 2-(2,6-dioxopiperidin-3-yl)-5,6-difluoroisoindoline-1,3-dion and tert-butyl 2,6-dimethylpiperazine-1-carboxylate were used to prepare the trifluoroacetate salt of the target compound (GT-03104) (yellow solid, 1.61 g, yield 81%). ¹H NMR (400 MHz, DMSO-d₆) δ 11.11 (s, 1H), 9.16 (d, J=12.0 Hz, 1H), 8.51 (d, J=12.0 Hz, 1H), 7.80 (d, J=8.0 Hz, 1H), 7.63 (d, J=8.0 Hz, 1H), 5.12 (dd, J=12.0, 4.0 Hz, 1H), 3.76 (d, J=12.0 Hz, 2H), 2.95-2.59 (m, 3H), 2.58-2.51 (m, 2H), 2.08-1.98 (m, 2H), 1.26 (d, J=6.5 Hz, 7H). LCMS (ESI) calcd for C₁₉H₃₂FN₄O₄⁺ [M+H]⁺, 389.41; found, 389.2.

### Example 12: Preparation of 2-(2,6-dioxopiperidin-3-yl)-5-fluoro-6-(piperazin-1-yl-2,2,3,3,5,5,6,6-d₈)isoindoline-1,3-dione (GT-03108)

Referring to Scheme 1, 2-(2,6-dioxopiperidin-3-yl)-5,6-difluoroisoindoline-1,3-dion and tert-butyl piperazine-1-carboxylate-2,2,3,3,5,5,6,6-d₈ were used to prepare the trifluoroacetate salt of the target compound (GT-03108) (yellow solid, 161.0 mg, yield 48%). ¹H NMR (400 MHz, DMSO-d₆) δ 11.11 (s, 1H), 8.79 (s, 2H), 7.80 (d, J=12.0 Hz, 1H), 7.59 (d, J=4.0 Hz, 1H), 5.12 (dd, J=12.0, 4.0 Hz, 1H), 2.93-2.84 (m, 1H), 2.67-2.51 (m, 2H), 2.07-2.01 (m, 1H). LCMS (ESI) calcd for C₁₇H₁₀D₈FN₄O₄⁺ [M+H]⁺, 369.40; found, 369.2.

### Example 13: Preparation of 5-(2,5-diazabicyclo[2.2.2]octan-2-yl)-2-(2,6-dioxopiperidin-3-yl)-6-fluoroisoindoline-1,3-dione (GT-03105)

Referring to Scheme 2, 2-(2,6-dioxopiperidin-3-yl)-5,6-difluoroisoindoline-1,3-dion and 2,5-diazabicyclo[2.2.2]octane were used to prepare the hydrochloride of the target compound (GT-03105) (brown solid, 200 mg, 44%). ¹H NMR (400 MHz, DMSO-d₆) δ 11.09 (s, 1H), 9.51 (s, 2H), 7.70 (d, J=12.0 Hz, 1H), 7.32 (d, J=8.0 Hz, 1H), 5.08 (dd, J=12.0, 4.0 Hz, 1H), 4.25 (s, 1H), 3.95 (d, J=12.0 Hz, 1H), 3.79 (s, 1H), 3.66 (d, J=12.0 Hz, 1H), 2.93-2.84 (m, 1H), 2.66-2.54 (m, 3H), 2.12-2.01 (m, 4H), 1.88-1.84 (m, 2H). LCMS (ESI) calcd for C₁₉H₂₀FN₄O₄⁺ [M+H]⁺, 387.39; found, 387.2.

### Intermediate Example 5: Preparation of N-((1r,4r)-4-(3-chloro-4-cyanophenoxy)cyclohexyl)-6-(4-formylpiperidin-1-yl)pyridazine-3-carboxamide

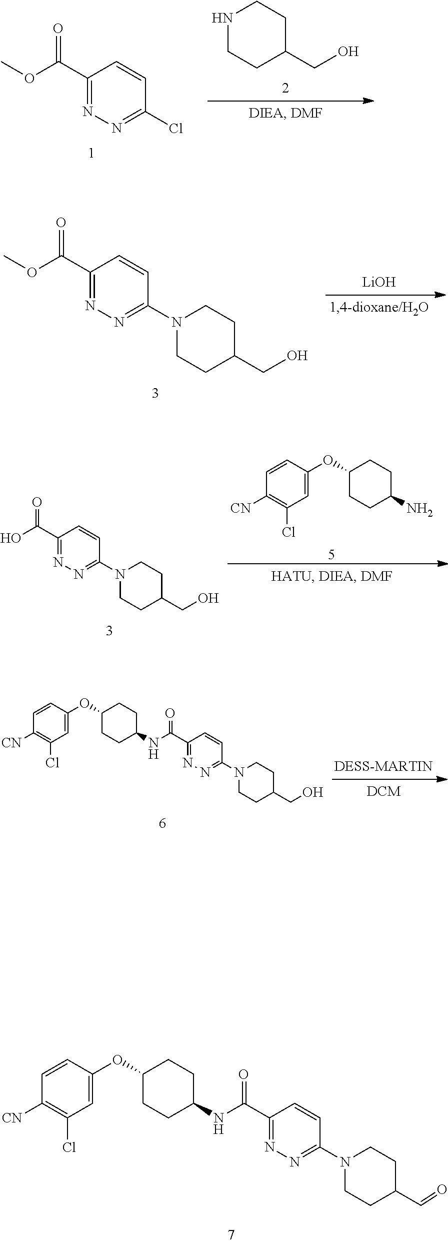

### Step 1: Preparation of methyl 6-(4-(hydroxymethyl)piperidin-1-yl)pyridazine-3-carboxylate

To a solution of methyl 6-chloropyridazine-3-carboxylate (1, 2.00 g, 11.589 mmol) and piperidin-4-ylmethanol (2, 1.406 mL, 12.169 mmol) in anhydrous DMF was added DIEA (5.746 mL, 34.768 mmol). The reaction solution was stirred at 60° C. for 6 h. After monitoring the reaction via LCMS and confirming its completion, water was added to the reaction solution. The resulting mixture was then extracted using ethyl acetate. The organic phases were combined, washed with saturated brine, and concentrated under reduced pressure to remove organic solvent. The resulting crude product was subjected to a silica gel column chromatography (eluent (v/v): PE to PE/EA (1/1)) for purification to give the compound methyl 6-(4-(hydroxymethyl)piperidin-1-yl)pyridazine-3-carboxylate (white solid, 2.50 g, yield 84.19%). LCMS (ESI) calcd for C₁₂H₁₈N₃O₃⁺ [M+H]⁺, 252.13; found, 252.20.

### Step 2: Preparation of 6-(4-(hydroxymethyl)piperidin-1-yl)pyridazine-3-carboxylic acid

To a mixed solution of methyl 6-(4-(hydroxymethyl)piperidin-1-yl)pyridazine-3-carboxylate (3, 2.5 g, 9.949 mmol) in 1,4-dioxane and water was added lithium hydroxide (1.68 g, 39.796 mmol). The reaction solution was stirred at room temperature for 5 h. After monitoring the reaction via LCMS and confirming its completion, the reaction solution was concentrated under reduced pressure to remove organic solvent. The pH of the reaction solution was adjusted to 6 using concentrated HCl. The reaction solution was lyophilized using a lyophilizer to remove water, yielding 6-(4-(hydroxymethyl)piperidin-1-yl)pyridazine-3-carboxylic acid (4, pale yellow solid, 2.00 g, yield 81.36%). LCMS (ESI) calcd for C₁₁H₁₆N₃O₃⁺ [M+H]⁺, 238.11; found, 238.10.

### Step 3: Preparation of N-((1r,4r)-4-(3-chloro-4-cyanophenoxy)cyclohexyl)-6-(4-(hydroxymethyl)piperidin-1-yl)pyridazine-3-carboxamide

To a mixed solution of 6-(4-(hydroxymethyl)piperidin-1-yl)pyridazine-3-carboxylic acid (4, 1.00 g, 2.950 mmol), HATU (1.68 g, 4.426 mmol) and DIEA (2.438 mL, 14.752 mmol) in anhydrous DMF was added 4-(((1r,4r)-4-aminocyclohexyl)oxy)-2-chlorobenzonitrile (5, 0.68 g, 2.360 mmol). The reaction solution was stirred at room temperature for 1 h. After monitoring the reaction via LCMS and confirming its completion, water was added to the reaction solution. The resulting mixture was then extracted using ethyl acetate. The organic phases were combined, washed with saturated brine, and concentrated under reduced pressure to remove organic solvent. The resulting crude product was subjected to a silica gel column chromatography (eluent (v/v): DCM to DCM/EA (50-70%)) for purification to give the compound N-((1r,4r)-4-(3-chloro-4-cyanophenoxy)cyclohexyl)-6-(4-(hydroxymethyl)piperidin-1-yl)pyridazine-3-carboxamide (6, white solid, 1.00 g, yield 64.75%).

LCMS (ESI) calcd for C₂₄H₂₉ClN₅O₃⁺ [M+H]⁺, 470.19; found, 470.20.

### Step 4: Preparation of N-((1r,4r)-4-(3-chloro-4-cyanophenoxy)cyclohexyl)-6-(4-formylpiperidin-1-yl)pyridazine-3-carboxamide

To a solution of N-((1r,4r)-4-(3-chloro-4-cyanophenoxy)cyclohexyl)-6-(4-(hydroxymethyl)piperidin-1-yl)pyridazine-3-carboxamide (6, 800 mg, 1.702 mmol) in DCM was added DESS-MARTIN (1.80 g, 4.256 mmol). The reaction solution was stirred at 0° C. for 1 h. After monitoring the reaction via LCMS and confirming its completion, saturated aqueous NaHCO₃ solution was added to the reaction solution. The resulting mixture was then extracted using DCM. The organic phases were combined, washed with saturated brine, and concentrated under reduced pressure to remove organic solvent. The resulting crude product was subjected to a silica gel column chromatography (eluent (v/v): DCM to DCM/EA (0-20%)) for purification to give the target compound N-((1r,4r)-4-(3-chloro-4-cyanophenoxy)cyclohexyl)-6-(4-formylpiperidin-1-yl)pyridazine-3-carboxamide (7, white solid, 690.00 mg, yield 82.29%). LCMS (ESI) calcd for C₂₄H₂₇ClN₅O₃⁺ [M+H]⁺, 468.17; found, 468.10.

### Example 14: Preparation of N-((1r,4r)-4-(3-chloro-4-cyanophenoxy)cyclohexyl)-6-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)pyridazine-3-carboxamide (GT-03016)

Referring to Scheme 4, N-((1r,4r)-4-(3-chloro-4-cyanophenoxy)cyclohexyl)-6-(4-formylpiperidin-1-yl)pyridazine-3-carboxamide prepared from intermediate example 15 and 2-(2,6-dioxopiperidin-3-yl)-5-fluoro-6-(piperazin-1-yl-2,2,3,3,5,5,6,6-d₈)isoindoline-1,3-dione (GT-03108) were used to prepare the target compound (GT-03016) (yellow solid, 65.1 mg, yield 37%). ¹H NMR (400 MHz, DMSO-d6) δ 11.11 (s, 1H), 10.12 (s, 1H), 8.57 (d, J=8.0 Hz, 1H), 7.86-7.80 (m, 3H), 7.59 (d, J=8.0 Hz, 1H), 7.41-7.37 (m, 2H), 7.13 (dd, J=8.0, 4.0 Hz, 1H), 5.75 (s, 1H), 5.12 (dd, J=12.0, 4.0 Hz, 1H), 4.52-4.48 (m, 3H), 3.87-3.86 (m, 1H), 3.10-3.04 (m, 3H), 2.94-2.85 (m, 2H), 2.69-2.54 (m, 2H), 2.24 (s, 1H), 2.12-2.01 (m, 4H), 1.91-1.91 (s, 4H), 1.64 (q, J=12.0 Hz, 2H), 1.52 (q, J=12.0 Hz, 2H). LCMS (ESI) calcd for C₄₁H₃₆D₈ClFN₉O₉⁺ [M+H]⁺, 821.36; found, 821.3/823.3.

### Example 15: Preparation of N-((1r,4r)-4-(3-chloro-4-cyanophenoxy)cyclohexyl)-6-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)pyridazine-3-carboxamide (GT-02765)

Referring to Scheme 4, N-((1r,4r)-4-(3-chloro-4-cyanophenoxy)cyclohexyl)-6-(4-formylpiperidin-1-yl)pyridazine-3-carboxamide and 5-(3,8-diazabicyclo[3.2.1]octan-8-yl)-2-(2,6-dioxopiperidin-3-yl)-6-fluoroisoindoline-1,3-dione (GT-03211) were used to prepare the target compound (GT-02765) (yellow solid, 45 mg, yield 49.24%). ¹H NMR (500 MHz, DMSO) δ 11.16 (s, 1H), 10.28 (s, 1H), 8.64 (d, J=8.2 Hz, 1H), 7.92-7.80 (m, 3H), 7.61 (d, J=7.5 Hz, 1H), 7.46-7.38 (m, 2H), 7.17 (dd, J=8.8, 2.4 Hz, 1H), 5.16 (dd, J=12.9, 5.4 Hz, 1H), 4.72 (s, 2H), 4.57 (ddd, J=14.2, 10.1, 3.9 Hz, 1H), 4.48 (d, J=13.0 Hz, 2H), 3.93-3.85 (m, 1H), 3.54 (s, 2H), 3.23 (dd, J=19.4, 9.6 Hz, 2H), 3.06 (t, J=11.8 Hz, 2H), 3.02-2.86 (m, 3H), 2.70-2.55 (m, 2H), 2.39 (d, J=7.8 Hz, 2H), 2.26 (s, 1H), 2.17-2.03 (m, 5H), 1.92 (d, J=11.9 Hz, 4H), 1.67 (dd, J=24.2, 10.5 Hz, 2H), 1.55 (dd, J=22.9, 9.8 Hz, 2H), 1.30 (dd, J=17.7, 7.7 Hz, 2H). LCMS (ESI) calcd for C₄₃H₄₆ClFN₉O₆⁺ [M+H]⁺: 838.32, found, 838.30.

### Example 16: Preparation of N-((1r,4r)-4-(3-chloro-4-cyanophenoxy)cyclohexyl)-6-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)pyridazine-3-carboxamide (GT-02752)

Referring to Scheme 4, N-((1r,4r)-4-(3-chloro-4-cyanophenoxy)cyclohexyl)-6-(4-formylpiperidin-1-yl)pyridazine-3-carboxamide and 5-(3,8-diazabicyclo[3.2.1]octan-3-yl)-2-(2,6-dioxopiperidin-3-yl)-6-fluoroisoindoline-1,3-dione (GT-03107) were used to prepare the target compound (GT-02752) (yellow solid, 20 mg, yield 21.88%). ¹H NMR (400 MHz, DMSO) δ 11.11 (s, 1H), 8.59 (d, J=8.2 Hz, 1H), 7.87-7.74 (m, 3H), 7.47 (d, J=7.2 Hz, 1H), 7.42-7.34 (m, 2H), 7.13 (dd, J=8.7, 2.2 Hz, 1H), 5.11 (dd, J=12.7, 5.3 Hz, 1H), 4.53 (dd, J=14.3, 11.0 Hz, 3H), 4.17 (s, 1H), 3.98 (d, J=12.6 Hz, 1H), 3.89-3.82 (m, 1H), 3.56 (d, J=12.4 Hz, 2H), 3.06 (t, J=12.1 Hz, 3H), 2.99-2.92 (m, 2H), 2.90-2.82 (m, 1H), 2.68-2.53 (m, 3H), 2.40-2.25 (m, 2H), 2.24-2.20 (m, 1H), 2.13-1.97 (m, 7H), 1.90 (d, J=12.2 Hz, 2H), 1.64 (dd, J=23.0, 10.9 Hz, 2H), 1.55-1.45 (m, 2H), 1.34-1.25 (m, 2H). LCMS (ESI) calcd for C₄₃H₄₆ClFN₉O₆⁺ [M+H]⁺: 838.32, found, 838.30.

### Example 17: Preparation of N-((1r,4r)-4-(3-chloro-4-cyanophenoxy)cyclohexyl)-6-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)pyridazine-3-carboxamide (GT-03339)

Referring to Scheme 4, N-((1r,4r)-4-(3-chloro-4-cyanophenoxy)cyclohexyl)-6-(4-formylpiperidin-1-yl)pyridazine-3-carboxamide and 5-(3,6-diazabicyclo[3.1.1]heptan-6-yl)-2-(2,6-dioxopiperidin-3-yl)isoindoline-1,3-dione (GT-03100) were used to prepare the target compound (GT-03339) (yellow solid, 30 mg, yield 56.88%). ¹H NMR (400 MHz, DMSO) δ 11.25 (s, 1H), 11.09 (s, 1H), 8.57 (d, J=8.2 Hz, 1H), 7.84 (dd, J=9.1, 5.7 Hz, 2H), 7.77 (d, J=8.2 Hz, 1H), 7.40 (dd, J=18.7, 5.9 Hz, 2H), 7.18-7.09 (m, 2H), 6.96 (d, J=8.2 Hz, 1H), 5.10 (dd, J=12.8, 5.4 Hz, 1H), 4.65 (s, 2H), 4.52 (td, J=9.7, 4.8 Hz, 1H), 4.41 (d, J=12.9 Hz, 2H), 3.85 (dd, J=7.6, 3.6 Hz, 1H), 3.77 (s, 2H), 3.39 (dt, J=12.6, 6.1 Hz, 2H), 3.06-2.86 (m, 3H), 2.86-2.72 (m, 4H), 2.65-2.52 (m, 2H), 2.10 (d, J=9.6 Hz, 3H), 2.01 (dd, J=8.9, 3.6 Hz, 1H), 1.89 (d, J=11.4 Hz, 4H), 1.63 (dd, J=24.2, 11.2 Hz, 2H), 1.50 (dd, J=22.6, 10.5 Hz, 2H), 1.19 (dt, J=14.7, 7.2 Hz, 2H). LCMS (ESI) calcd for C₄₂H₄₅ClN₉O₆⁺ [M+H]⁺: 806.31, found, 806.30.

### Example 18: Preparation of N-((1r,3r)-3-(3-chloro-4-cyanophenoxy)-2,2,4,4-tetramethylcyclobutyl)-6-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)pyridazine-3-carboxamide (GT-03398)

Referring to the method of Scheme 4, the target compound (GT-03398) was prepared, which is a yellow solid (44 mg, yield 49.37%). ¹H NMR (400 MHz, DMSO) δ 11.12 (s, 1H), 10.27 (s, 1H), 8.23 (d, J=9.2 Hz, 1H), 7.90 (d, J=8.8 Hz, 1H), 7.81 (dd, J=15.7, 10.9 Hz, 2H), 7.58 (d, J=7.4 Hz, 1H), 7.40 (d, J=9.7 Hz, 1H), 7.24 (d, J=2.4 Hz, 1H), 7.03 (dd, J=8.8, 2.4 Hz, 1H), 5.12 (dd, J=12.8, 5.3 Hz, 1H), 4.69 (s, 2H), 4.47 (d, J=9.2 Hz, 3H), 4.00 (d, J=9.1 Hz, 1H), 3.47 (s, 6H), 3.26-3.13 (m, 2H), 2.99 (dt, J=12.9, 10.3 Hz, 4H), 2.63-2.56 (m, 1H), 2.36 (d, J=7.7 Hz, 2H), 2.27-2.17 (m, 1H), 2.13-1.98 (m, 3H), 1.89 (d, J=12.2 Hz, 2H), 1.21 (s, 6H), 1.14 (s, 6H).

LCMS (ESI) calcd for C₄₅H₅₀ClFN₉O₆⁺ [M+H]⁺: 866.35, found, 866.30.

### Example 19: Preparation of N-((1r,3r)-3-(3-chloro-4-cyanophenoxy)-2,2,4,4-tetramethylcyclobutyl)-6-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)pyridazine-3-carboxamide (GT-03399)

Referring to the method of Scheme 4, the target compound (GT-03399) was prepared, which is a yellow solid (40 mg, yield 45.84%). ¹H NMR (400 MHz, DMSO) δ 11.10 (s, 1H), 10.66 (s, 1H), 8.24 (d, J=9.3 Hz, 1H), 7.91 (d, J=8.7 Hz, 1H), 7.85 (dd, J=9.6, 4.1 Hz, 1H), 7.76 (d, J=8.4 Hz, 1H), 7.47-7.34 (m, 2H), 7.28-7.22 (m, 2H), 7.03 (dd, J=8.8, 2.3 Hz, 1H), 5.09 (dd, J=12.7, 5.4 Hz, 1H), 4.55 (d, J=12.4 Hz, 2H), 4.46 (s, 1H), 4.20 (s, 2H), 4.08-3.89 (m, 3H), 3.72 (d, J=12.2 Hz, 2H), 3.09 (t, J=12.4 Hz, 2H), 2.97 (dd, J=13.0, 7.5 Hz, 2H), 2.93-2.84 (m, 1H), 2.67-2.52 (m, 2H), 2.36-2.18 (m, 3H), 2.06-1.92 (m, 5H), 1.32 (ddd, J=21.3, 12.1, 7.0 Hz, 2H), 1.22 (s, 6H), 1.14 (s, 6H). LCMS (ESI) calcd for C₄₅H₅₁ClN₉O₆⁺ [M+H]⁺: 848.36, found, 848.30.

### Example 20: Preparation of N-((1r,3r)-3-(3-chloro-4-cyanophenoxy)-2,2,4,4-tetramethylcyclobutyl)-6-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)pyridazine-3-carboxamide (GT-03400)

Referring to the method of Scheme 4, the target compound (GT-03400) was prepared, which is a yellow solid (42 mg, yield 48.94%). ¹H NMR (400 MHz, DMSO) δ 11.35 (s, 1H), 11.10 (s, 1H), 8.22 (t, J=8.4 Hz, 1H), 7.90 (d, J=8.8 Hz, 1H), 7.84 (d, J=9.6 Hz, 1H), 7.77 (d, J=8.2 Hz, 1H), 7.43 (d, J=9.7 Hz, 1H), 7.23 (d, J=2.3 Hz, 1H), 7.16 (d, J=1.3 Hz, 1H), 7.02 (dd, J=8.8, 2.3 Hz, 1H), 6.96 (dd, J=8.2, 1.7 Hz, 1H), 5.10 (dd, J=12.8, 5.3 Hz, 1H), 4.65 (d, J=4.2 Hz, 2H), 4.43 (d, J=12.0 Hz, 3H), 4.00-3.97 (m, 1H), 3.77 (dd, J=12.3, 4.1 Hz, 4H), 3.44-3.34 (m, 2H), 3.02-2.88 (m, 3H), 2.86-2.74 (m, 4H), 2.56 (dd, J=21.7, 11.4 Hz, 2H), 2.19-2.08 (m, 1H), 2.00 (dd, J=8.8, 3.7 Hz, 1H), 1.90 (d, J=11.5 Hz, 2H), 1.21 (s, 6H), 1.13 (s, 6H). LCMS (ESI) calcd for C₄₄H₄₉ClN₉O₆⁺ [M+H]⁺: 834.34, found, 834.30.

### Example 21: Preparation of N-((1r,3r)-3-(3-chloro-4-cyanophenoxy)-2,2,4,4-tetramethylcyclobutyl)-6-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)pyridazine-3-carboxamide (GT-03401)

Referring to the method of Scheme 4, the target compound (GT-03401) was prepared, which is a yellow solid (47 mg, yield 54.76%). ¹H NMR (400 MHz, DMSO) δ 11.35 (s, 1H), 11.10 (s, 1H), 8.22 (t, J=8.4 Hz, 1H), 7.90 (d, J=8.8 Hz, 1H), 7.84 (d, J=9.6 Hz, 1H), 7.77 (d, J=8.2 Hz, 1H), 7.43 (d, J=9.7 Hz, 1H), 7.23 (d, J=2.3 Hz, 1H), 7.16 (d, J=1.3 Hz, 1H), 7.02 (dd, J=8.8, 2.3 Hz, 1H), 6.96 (dd, J=8.2, 1.7 Hz, 1H), 5.10 (dd, J=12.8, 5.3 Hz, 1H), 4.65 (d, J=4.2 Hz, 2H), 4.43 (d, J=12.0 Hz, 3H), 4.00-3.97 (m, 1H), 3.77 (dd, J=12.3, 4.1 Hz, 4H), 3.44-3.34 (m, 2H), 3.02-2.88 (m, 3H), 2.86-2.74 (m, 4H), 2.56 (dd, J=21.7, 11.4 Hz, 2H), 2.19-2.08 (m, 1H), 2.00 (dd, J=8.8, 3.7 Hz, 1H), 1.90 (d, J=11.5 Hz, 2H), 1.21 (s, 6H), 1.13 (s, 6H). LCMS (ESI) calcd for C₄₄H₄₉ClN₉O₆⁺ [M+H]⁺: 834.34, found, 834.30.

### Example 22: Preparation of N-((1r,3r)-3-(3-chloro-4-cyanophenoxy)-2,2,4,4-tetramethylcyclobutyl)-6-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)nicotinamide (GT-03402)

Referring to the method of Scheme 4, the target compound (GT-03402) was prepared, which is a yellow solid (51 mg, yield 58.35%). ¹H NMR (400 MHz, DMSO) δ 11.12 (s, 1H), 10.60 (s, 1H), 8.57 (d, J=1.8 Hz, 1H), 8.15 (d, J=8.8 Hz, 1H), 7.91 (t, J=9.2 Hz, 2H), 7.78 (d, J=12.1 Hz, 1H), 7.57 (d, J=7.5 Hz, 1H), 7.18 (dd, J=13.9, 6.1 Hz, 2H), 7.00 (dd, J=8.8, 2.3 Hz, 1H), 5.12 (dd, J=12.8, 5.4 Hz, 1H), 4.68 (s, 2H), 4.40 (d, J=12.8 Hz, 2H), 4.34 (s, 1H), 4.06 (d, J=9.2 Hz, 1H), 3.49 (d, J=10.7 Hz, 2H), 3.21-3.05 (m, 4H), 2.98-2.84 (m, 3H), 2.67-2.51 (m, 2H), 2.42 (d, J=7.6 Hz, 2H), 2.25 (s, 1H), 2.13-2.00 (m, 3H), 1.91 (d, J=12.3 Hz, 2H), 1.29 (dd, J=17.1, 6.1 Hz, 2H), 1.22 (s, 6H), 1.12 (s, 6H). LCMS (ESI) calcd for C₄₆H₅₁ClFN₈O₆⁺ [M+H]⁺: 865.35, found, 865.30.

### Example 23: Preparation of N-((1r,3r)-3-(3-chloro-4-cyanophenoxy)-2,2,4,4-tetramethylcyclobutyl)-6-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)nicotinamide (GT-03403)

Referring to the method of Scheme 4, the target compound (GT-03403) was prepared, which is a yellow solid (51 mg, yield 58.35%). ¹H NMR (400 MHz, DMSO) δ 11.27 (s, 1H), 11.12 (s, 1H), 8.55 (d, J=1.9 Hz, 1H), 8.14 (d, J=8.5 Hz, 1H), 7.91 (t, J=10.7 Hz, 2H), 7.77 (d, J=10.9 Hz, 1H), 7.33 (d, J=7.3 Hz, 1H), 7.18 (dd, J=18.6, 5.3 Hz, 2H), 7.00 (dd, J=8.8, 2.3 Hz, 1H), 5.11 (dd, J=12.8, 5.4 Hz, 1H), 4.65 (s, 2H), 4.41-4.32 (m, 3H), 4.06 (d, J=9.2 Hz, 1H), 3.85 (d, J=8.6 Hz, 3H), 3.38 (dd, J=11.3, 6.6 Hz, 3H), 3.04 (t, J=12.3 Hz, 2H), 2.94-2.80 (m, 5H), 2.65-2.51 (m, 2H), 2.19 (s, 1H), 2.03 (d, J=5.6 Hz, 1H), 1.94 (d, J=10.8 Hz, 2H), 1.22 (s, 6H), 1.12 (s, 6H). LCMS (ESI) calcd for C₄₅H₄₉ClFN₈O₆⁺ [M+H]⁺: 851.34, found, 851.30.

### Example 24: Preparation of N-((1r,3r)-3-(3-chloro-4-cyanophenoxy)-2,2,4,4-tetramethylcyclobutyl)-6-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)nicotinamide (GT-03404)

Referring to the method of Scheme 4, the target compound (GT-03404) was prepared, which is a yellow solid (13 mg, yield 14.82%). ¹H NMR (400 MHz, DMSO) δ 11.09 (d, J=15.3 Hz, 2H), 8.55 (d, J=2.0 Hz, 1H), 8.10 (d, J=8.8 Hz, 1H), 7.88 (dd, J=21.0, 7.8 Hz, 2H), 7.77 (d, J=10.9 Hz, 1H), 7.33 (d, J=7.3 Hz, 1H), 7.21 (d, J=2.3 Hz, 1H), 7.10 (d, J=5.2 Hz, 1H), 7.00 (dd, J=8.8, 2.3 Hz, 1H), 5.11 (dd, J=12.8, 5.4 Hz, 1H), 4.65 (s, 2H), 4.40-4.32 (m, 3H), 4.05 (d, J=9.1 Hz, 1H), 3.84 (d, J=10.2 Hz, 2H), 3.39 (d, J=9.2 Hz, 2H), 3.01 (t, J=12.0 Hz, 2H), 2.96-2.70 (m, 6H), 2.62-2.56 (m, 1H), 2.21-2.15 (m, 1H), 2.05-1.99 (m, 1H), 1.96-1.88 (m, 2H), 1.22 (d, J=5.5 Hz, 8H), 1.11 (s, 6H). LCMS (ESI) calcd for C₄₅H₄₉ClFN₈O₆⁺ [M+H]⁺: 851.34, found, 851.30.

### Example 25: Preparation of N-((1r,3r)-3-(3-chloro-4-cyanophenoxy)-2,2,4,4-tetramethylcyclobutyl)-6-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)nicotinamide (GT-03425)

Referring to the method of Scheme 4, the target compound (GT-03425) was prepared, which is a yellow solid (41 mg, yield 47.90%). ¹H NMR (400 MHz, DMSO) δ 11.09 (s, 1H), 10.50 (s, 1H), 8.57 (d, J=1.8 Hz, 1H), 8.10 (d, J=8.6 Hz, 1H), 7.88 (dd, J=13.9, 8.9 Hz, 2H), 7.75 (d, J=8.4 Hz, 1H), 7.40 (s, 1H), 7.27 (d, J=7.7 Hz, 1H), 7.20 (d, J=2.3 Hz, 1H), 7.09 (d, J=6.9 Hz, 1H), 7.00 (dd, J=8.8, 2.3 Hz, 1H), 5.09 (dd, J=12.9, 5.3 Hz, 1H), 4.77 (s, 2H), 4.41-4.30 (m, 3H), 4.05 (d, J=9.2 Hz, 1H), 3.43 (d, J=10.6 Hz, 2H), 3.06 (d, J=10.1 Hz, 4H), 2.93-2.82 (m, 3H), 2.66-2.53 (m, 2H), 2.43 (d, J=7.6 Hz, 2H), 2.26-2.16 (m, 1H), 2.12-1.98 (m, 3H), 1.87 (d, J=11.6 Hz, 2H), 1.22 (s, 8H), 1.11 (s, 6H). LCMS (ESI) calcd for C₄₆H₅₂ClN₈O₆⁺ [M+H]⁺: 847.36, found, 847.30.

### Example 26: Preparation of N-((1r,3r)-3-(3-chloro-4-cyanophenoxy)-2,2,4,4-tetramethylcyclobutyl)-6-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)nicotinamide (GT-03426)

Referring to the method of Scheme 4, the target compound (GT-03426) was prepared, which is a yellow solid (30 mg, yield 35.05%). ¹H NMR (400 MHz, DMSO) δ 11.09 (s, 1H), 10.65 (s, 1H), 8.62 (d, J=1.7 Hz, 1H), 8.05 (d, J=8.5 Hz, 1H), 7.91 (d, J=8.7 Hz, 1H), 7.75 (t, J=7.6 Hz, 2H), 7.38 (d, J=17.2 Hz, 1H), 7.23 (dd, J=18.2, 4.7 Hz, 2H), 7.10-6.96 (m, 2H), 5.09 (dd, J=12.9, 5.3 Hz, 1H), 4.47 (d, J=13.3 Hz, 2H), 4.33 (s, 1H), 4.20 (s, 2H), 4.06 (d, J=9.2 Hz, 1H), 4.00 (d, J=12.5 Hz, 2H), 3.72 (d, J=12.1 Hz, 2H), 3.09-2.92 (m, 4H), 2.87 (dd, J=16.8, 5.3 Hz, 1H), 2.61 (ddd, J=27.2, 8.2, 3.3 Hz, 2H), 2.34-2.19 (m, 3H), 2.06-1.91 (m, 5H), 1.31-1.21 (m, 8H), 1.12 (s, 6H). LCMS (ESI) calcd for C₄₆H₅₂ClN₈O₆⁺ [M+H]⁺: 847.36, found, 847.30.

### Example 27: Preparation of N-((1r,3r)-3-(3-chloro-4-cyanophenoxy)-2,2,4,4-tetramethylcyclobutyl)-6-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)nicotinamide (GT-03427)

Referring to the method of Scheme 4, the target compound (GT-03427) was prepared, which is a yellow solid (46 mg, yield 54.64%). ¹H NMR (400 MHz, DMSO) δ 11.28 (s, 1H), 11.10 (s, 1H), 8.56 (d, J=1.7 Hz, 1H), 8.13 (d, J=8.7 Hz, 1H), 7.90 (d, J=8.7 Hz, 2H), 7.77 (d, J=8.2 Hz, 1H), 7.22-7.11 (m, 3H), 7.02-6.93 (m, 2H), 5.10 (dd, J=12.8, 5.3 Hz, 1H), 4.65 (d, J=2.6 Hz, 2H), 4.41-4.31 (m, 3H), 4.06 (d, J=9.1 Hz, 1H), 3.77 (d, J=8.7 Hz, 2H), 3.38 (dd, J=11.9, 5.9 Hz, 2H), 3.00 (t, J=12.0 Hz, 2H), 2.92-2.73 (m, 5H), 2.56 (dd, J=22.1, 11.2 Hz, 2H), 2.13 (dd, J=16.1, 9.7 Hz, 1H), 2.04-1.98 (m, 1H), 1.91 (d, J=11.6 Hz, 2H), 1.22 (s, 8H), 1.12 (s, 6H). LCMS (ESI) calcd for C₄₅H₅₀ClN₈O₆⁺ [M+H]⁺: 833.35, found, 833.40.

### Example 28: Preparation of N-((1r,4r)-4-(3-chloro-4-cyanophenoxy)cyclohexyl)-6-(3-((1-(2-(2,6-dioxopiperidin-3-yl)-6-fluoro-1,3-dioxoisoindolin-5-yl)piperidin-4-yl)methyl)-3,8-diazabicyclo[3.2.1]octan-8-yl)pyridazine-3-carboxamide (GT-03449)

Referring to the method of Scheme 4, the target compound (GT-03449) was prepared, which is a yellow solid (37 mg, yield 34.19%). ¹H NMR (400 MHz, DMSO) δ 11.10 (s, 1H), 10.42 (s, 1H), 8.70 (d, J=8.2 Hz, 1H), 7.96 (d, J=9.3 Hz, 1H), 7.85 (d, J=8.8 Hz, 1H), 7.70 (d, J=11.4 Hz, 1H), 7.40 (dd, J=18.6, 5.2 Hz, 3H), 7.13 (dd, J=8.8, 2.3 Hz, 1H), 5.10 (dd, J=12.7, 5.3 Hz, 1H), 4.90 (s, 2H), 4.53 (ddd, J=14.0, 10.0, 4.0 Hz, 1H), 3.96-3.81 (m, 1H), 3.54 (dd, J=25.2, 11.6 Hz, 4H), 3.14 (t, J=10.2 Hz, 2H), 2.90 (dd, J=25.8, 13.0 Hz, 4H), 2.58 (dd, J=24.1, 7.2 Hz, 2H), 2.47-2.36 (m, 2H), 2.13-1.99 (m, 6H), 1.88 (s, 4H), 1.65 (d, J=12.6 Hz, 2H), 1.52 (dd, J=22.5, 10.2 Hz, 2H), 1.38 (dd, J=22.4, 11.6 Hz, 2H). LCMS (ESI) calcd for C₄₃H₄₆ClFN₉O₆⁺ [M+H]⁺: 838.32, found, 838.30.

### Example 29: Preparation of N-((1r,4r)-4-(3-chloro-4-cyanophenoxy)cyclohexyl)-6-(8-((1-(2-(2,6-dioxopiperidin-3-yl)-6-fluoro-1,3-dioxoisoindolin-5-yl)piperidin-4-yl)methyl)-3,8-diazabicyclo[3.2.1]octan-3-yl)pyridazine-3-carboxamide (GT-03450)

Referring to the method of Scheme 4, the target compound (GT-03450) was prepared, which is a yellow solid (35 mg, yield 32.36%). ¹H NMR (400 MHz, DMSO) δ 11.11 (s, 2H), 8.67 (d, J=8.1 Hz, 1H), 7.99-7.89 (m, 1H), 7.85 (d, J=8.8 Hz, 1H), 7.73 (d, J=11.4 Hz, 1H), 7.48 (d, J=7.4 Hz, 1H), 7.44-7.34 (m, 2H), 7.13 (dd, J=8.8, 2.2 Hz, 1H), 5.11 (dd, J=12.8, 5.3 Hz, 1H), 4.60-4.47 (m, 1H), 4.37-4.10 (m, 4H), 4.00-3.70 (m, 3H), 3.66 (d, J=11.2 Hz, 2H), 3.08-2.84 (m, 5H), 2.66-2.52 (m, 2H), 2.37-2.17 (m, 3H), 2.14-2.01 (m, 5H), 1.90 (d, J=8.4 Hz, 4H), 1.65 (dd, J=24.3, 11.7 Hz, 2H), 1.56-1.39 (m, 4H). LCMS (ESI) calcd for C₄₃H₄₆ClFN₉O₆⁺ [M+H]⁺: 838.32, found, 838.30.

### Example 30: Preparation of N-((1r,4r)-4-(3-chloro-4-cyanophenoxy)cyclohexyl)-6-(3-((1-(2-(2,6-dioxopiperidin-3-yl)-6-fluoro-1,3-dioxoisoindolin-5-yl)piperidin-4-yl)methyl)-3,6-diazabicyclo[3.1.1]heptan-6-yl)pyridazine-3-carboxamide (GT-03451)

Referring to the method of Scheme 4, the target compound (GT-03451) was prepared, which is a yellow solid (46.00 mg, yield 43.26%). ¹H NMR (400 MHz, DMSO) δ 11.36 (s, 1H), 11.10 (s, 1H), 8.69 (d, J=8.2 Hz, 1H), 8.00 (d, J=9.2 Hz, 1H), 7.85 (d, J=8.8 Hz, 1H), 7.69 (d, J=11.4 Hz, 1H), 7.39 (dd, J=14.0, 4.9 Hz, 2H), 7.22 (d, J=9.2 Hz, 1H), 7.12 (dd, J=8.8, 2.4 Hz, 1H), 5.09 (dd, J=12.8, 5.4 Hz, 1H), 4.66 (d, J=5.7 Hz, 2H), 4.52 (td, J=10.2, 5.0 Hz, 1H), 3.90-3.78 (m, 3H), 3.54 (d, J=12.4 Hz, 3H), 2.97-2.83 (m, 4H), 2.76 (t, J=10.0 Hz, 3H), 2.57 (dd, J=23.6, 7.5 Hz, 2H), 2.18-1.96 (m, 4H), 1.86 (d, J=10.4 Hz, 5H), 1.63 (dd, J=23.8, 10.9 Hz, 2H), 1.54-1.45 (m, 2H), 1.34 (dd, J=22.1, 13.0 Hz, 2H). LCMS (ESI) calcd for C₄₂H₄₄ClFN₉O₆⁺ [M+H]⁺: 824.30, found, 824.30.

### Example 31: Preparation of N-((1r,4r)-4-(3-chloro-4-cyanophenoxy)cyclohexyl)-6-(6-((1-(2-(2,6-dioxopiperidin-3-yl)-6-fluoro-1,3-dioxoisoindolin-5-yl)piperidin-4-yl)methyl)-3,6-diazabicyclo[3.1.1]heptan-3-yl)pyridazine-3-carboxamide (GT-03470)

Referring to the method of Scheme 4, the target compound (GT-03470) was prepared, which is a yellow solid (55.00 mg, yield 51.72%). ¹H NMR (400 MHz, DMSO) δ 11.10 (d, J=2.0 Hz, 1H), 8.70-8.64 (m, 1H), 7.98 (dd, J=9.4, 3.3 Hz, 1H), 7.85 (dd, J=8.8, 1.9 Hz, 1H), 7.72 (dd, J=8.8, 5.8 Hz, 1H), 7.50-7.45 (m, 1H), 7.38 (d, J=1.4 Hz, 1H), 7.24 (dd, J=9.5, 6.9 Hz, 1H), 7.15-7.12 (m, 1H), 5.12-5.08 (m, 1H), 4.56-4.51 (m, 2H), 4.22-4.08 (m, 2H), 4.00 (d, J=13.7 Hz, 1H), 3.90-3.84 (m, 1H), 3.64 (dd, J=21.8, 9.6 Hz, 3H), 2.92 (dd, J=17.1, 6.5 Hz, 4H), 2.59 (d, J=17.9 Hz, 2H), 2.11 (d, J=9.9 Hz, 3H), 2.05-1.98 (m, 3H), 1.89 (s, 4H), 1.66 (dd, J=23.9, 12.1 Hz, 3H), 1.56-1.38 (m, 5H). LCMS (ESI) calcd for C₄₂H₄₄ClFN₉O₆⁺ [M+H]⁺: 824.30, found, 824.30.

### Example 32: Preparation of N-((1r,3r)-3-(3-chloro-4-cyanophenoxy)-2,2,4,4-tetramethylcyclobutyl)-6-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)nicotinamide (GT-03471)

Referring to the method of Scheme 4, the target compound (GT-03471) was prepared, which is a yellow solid (33 mg, yield 39.20%). ¹H NMR (400 MHz, DMSO) δ 11.09 (d, J=5.3 Hz, 1H), 8.63-8.52 (m, 1H), 8.13 (t, J=9.1 Hz, 1H), 7.90 (d, J=8.7 Hz, 2H), 7.86-7.72 (m, 1H), 7.17 (dd, J=21.7, 13.4 Hz, 3H), 7.00 (dd, J=8.7, 1.9 Hz, 1H), 5.09 (dt, J=11.2, 5.4 Hz, 1H), 4.65 (s, 1H), 4.47 (dd, J=17.6, 8.6 Hz, 2H), 4.34 (d, J=2.9 Hz, 1H), 4.15-4.04 (m, 2H), 4.00 (d, J=13.2 Hz, 1H), 3.92 (d, J=12.9 Hz, 1H), 3.76 (dd, J=11.7, 3.6 Hz, 2H), 3.44-3.35 (m, 2H), 3.17-2.96 (m, 3H), 2.83 (dd, J=31.1, 14.3 Hz, 3H), 2.59 (d, J=18.7 Hz, 2H), 2.18 (t, J=7.4 Hz, 1H), 2.10-1.78 (m, 4H), 1.32-1.22 (m, 8H), 1.12 (s, 6H). LCMS (ESI) calcd for C₄₅H₅₀ClN₈O₆⁺ [M+H]⁺: 833.35, found, 833.30.

### Example 33: Preparation of N-((1r,3r)-3-(3-chloro-4-cyanophenoxy)-2,2,4,4-tetramethylcyclobutyl)-6-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)nicotinamide (GT-03481)

Referring to the method of Scheme 4, the target compound (GT-03481) was prepared, which is a yellow solid (39.00 mg, yield 45.56%). ¹H NMR (400 MHz, DMSO) δ 11.08 (s, 1H), 10.78 (s, 1H), 8.59 (s, 1H), 8.11 (d, J=8.3 Hz, 1H), 7.88 (dd, J=20.8, 8.2 Hz, 2H), 7.72 (dd, J=8.4, 5.2 Hz, 1H), 7.26-7.08 (m, 3H), 7.01 (dd, J=8.7, 2.2 Hz, 2H), 5.07 (dd, J=12.8, 5.5 Hz, 1H), 4.57 (s, 1H), 4.44 (d, J=11.0 Hz, 2H), 4.33 (s, 1H), 4.06 (d, J=9.2 Hz, 1H), 3.98 (d, J=8.8 Hz, 1H), 3.86 (d, J=9.8 Hz, 1H), 3.26-3.19 (m, 2H), 3.07 (t, J=11.7 Hz, 2H), 2.93-2.84 (m, 1H), 2.63-2.55 (m, 2H), 2.22 (dd, J=18.1, 9.9 Hz, 1H), 1.99 (dd, J=21.5, 8.7 Hz, 4H), 1.84 (d, J=3.9 Hz, 1H), 1.29 (d, J=11.5 Hz, 2H), 1.22 (s, 6H), 1.12 (s, 6H). LCMS (ESI) calcd for C₄₆H₅₂ClN₈O₆⁺ [M+H]⁺: 847.36, found, 847.40.

### Example 34: Preparation of N-((1r,3r)-3-(3-chloro-4-cyanophenoxy)-2,2,4,4-tetramethylcyclobutyl)-6-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)nicotinamide (GT-03482)

Referring to the method of Scheme 4, the target compound (GT-03482) was prepared, which is a yellow solid (35.00 mg, yield 39.24%). ¹H NMR (400 MHz, DMSO) δ 11.12 (s, 1H), 10.92 (s, 1H), 8.60 (d, J=2.0 Hz, 1H), 8.16 (d, J=7.8 Hz, 1H), 7.91 (t, J=7.0 Hz, 2H), 7.80 (d, J=11.5 Hz, 1H), 7.48 (d, J=7.3 Hz, 1H), 7.20 (t, J=7.8 Hz, 2H), 7.01 (dd, J=8.8, 2.4 Hz, 1H), 5.12 (dd, J=12.8, 5.4 Hz, 1H), 4.48 (d, J=13.0 Hz, 2H), 4.35 (s, 1H), 4.18 (s, 2H), 4.07 (d, J=9.2 Hz, 1H), 3.95 (d, J=12.5 Hz, 2H), 3.58 (d, J=11.3 Hz, 2H), 3.12 (t, J=11.9 Hz, 2H), 3.02-2.85 (m, 3H), 2.66-2.51 (m, 2H), 2.28 (ddd, J=27.6, 7.6, 3.1 Hz, 3H), 2.09-1.98 (m, 5H), 1.38-1.28 (m, 2H), 1.23 (s, 6H), 1.13 (s, 6H). LCMS (ESI) calcd for C₄₆H₅₁ClFN₈O₆⁺ [M+H]⁺: 865.35, found, 865.40.

### Example 35: Preparation of N-((1r,4r)-4-(3-chloro-4-cyanophenoxy)cyclohexyl)-6-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)pyridazine-3-carboxamide (GT-03486)

Referring to the method of Scheme 4, the target compound (GT-03486) was prepared, which is a yellow solid (66 mg, yield 74.19%). ¹H NMR (400 MHz, DMSO) δ 11.34 (s, 1H), 11.11 (s, 1H), 8.58 (d, J=8.2 Hz, 1H), 7.86-7.82 (m, 2H), 7.76 (d, J=10.9 Hz, 1H), 7.45 (d, J=9.7 Hz, 1H), 7.37 (d, J=2.3 Hz, 1H), 7.32 (d, J=7.3 Hz, 1H), 7.12 (dd, J=8.8, 2.4 Hz, 1H), 5.11 (dd, J=12.7, 5.4 Hz, 1H), 4.65 (s, 2H), 4.53 (dd, J=9.1, 5.1 Hz, 1H), 4.42 (d, J=13.2 Hz, 2H), 3.37 (dd, J=14.0, 10.5 Hz, 2H), 3.00 (t, J=12.1 Hz, 2H), 2.94-2.80 (m, 5H), 2.62-2.52 (m, 2H), 2.26-2.16 (m, 1H), 2.10 (d, J=10.0 Hz, 2H), 2.01 (dd, J=11.3, 6.1 Hz, 1H), 1.91 (t, J=13.5 Hz, 4H), 1.63 (d, J=12.9 Hz, 2H), 1.54-1.46 (m, 2H), 1.22 (dd, J=20.0, 10.3 Hz, 2H). LCMS (ESI) calcd for C₄₂H₄₄ClFN₉O₆⁺ [M+H]⁺: 824.30, found, 824.30.

### Example 36: Preparation of N-((1r,4r)-4-(3-chloro-4-cyanophenoxy)cyclohexyl)-6-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)pyridazine-3-carboxamide (GT-03487)

Referring to the method of Scheme 4, the target compound (GT-03487) was prepared, which is a yellow solid (39 mg, yield 44.22%). ¹H NMR (400 MHz, DMSO) δ 11.39 (s, 1H), 11.11 (s, 1H), 8.58 (d, J=8.1 Hz, 1H), 7.83 (t, J=4.8 Hz, 2H), 7.76 (d, J=10.9 Hz, 1H), 7.43 (d, J=9.8 Hz, 1H), 7.37 (s, 1H), 7.32 (d, J=7.3 Hz, 1H), 7.13 (d, J=2.2 Hz, 1H), 5.11 (dd, J=12.8, 5.4 Hz, 1H), 4.64 (s, 1H), 4.54-4.50 (m, 2H), 4.41 (s, 1H), 3.38 (d, J=0.8 Hz, 1H), 3.01 (dd, J=21.9, 9.6 Hz, 3H), 2.89 (d, J=2.2 Hz, 2H), 2.59 (d, J=18.6 Hz, 2H), 2.25-2.19 (m, 1H), 2.10 (d, J=11.1 Hz, 3H), 2.02 (dd, J=9.0, 3.8 Hz, 2H), 1.89 (d, J=12.4 Hz, 4H), 1.67-1.46 (m, 8H), 1.30-1.20 (m, 3H).

LCMS (ESI) calcd for C₄₂H₄₄ClFN₉O₆⁺ [M+H]⁺: 824.30, found, 824.30.

### Example 37: Preparation of N-((1r,4r)-4-(3-chloro-4-cyanophenoxy)cyclohexyl)-6-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)pyridazine-3-carboxamide (GT-03488)

Referring to the method of Scheme 4, the target compound (GT-03488) was prepared, which is a yellow solid (71 mg, yield 80.88%). ¹H NMR (400 MHz, DMSO) δ 11.09 (s, 1H), 10.34 (s, 1H), 8.58 (d, J=8.2 Hz, 1H), 7.88-7.80 (m, 2H), 7.75 (d, J=8.4 Hz, 1H), 7.39 (dd, J=9.6, 4.1 Hz, 3H), 7.26 (dd, J=9.6, 7.9 Hz, 1H), 7.13 (dd, J=8.8, 2.4 Hz, 1H), 5.09 (dd, J=12.9, 5.4 Hz, 1H), 4.77 (s, 2H), 4.59-4.49 (m, 1H), 4.41 (d, J=13.2 Hz, 2H), 3.84 (dt, J=9.6, 6.5 Hz, 1H), 3.43 (d, J=10.4 Hz, 2H), 3.04 (dd, J=24.0, 12.3 Hz, 4H), 2.97-2.83 (m, 3H), 2.68-2.53 (m, 2H), 2.40 (d, J=7.7 Hz, 2H), 2.21 (s, 1H), 2.14-1.99 (m, 5H), 1.88 (t, J=10.9 Hz, 4H), 1.67-1.48 (m, 4H), 1.22 (dd, J=22.2, 11.4 Hz, 2H). LCMS (ESI) calcd for C₄₃H₄₇ClN₉O₆⁺ [M+H]⁺: 820.33, found, 820.30.

### Example 38: Preparation of N-((1r,4r)-4-(3-chloro-4-cyanophenoxy)cyclohexyl)-6-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)pyridazine-3-carboxamide (GT-03489)

Referring to the method of Scheme 4, the target compound (GT-03489) was prepared, which is a yellow solid (72 mg, yield 82.02%). ¹H NMR (400 MHz, DMSO) δ 11.39 (s, 1H), 11.09 (s, 1H), 8.63 (d, J=8.1 Hz, 1H), 7.95 (d, J=9.6 Hz, 1H), 7.84 (d, J=8.8 Hz, 1H), 7.77-7.63 (m, 2H), 7.35 (t, J=5.9 Hz, 2H), 7.24 (d, J=8.6 Hz, 1H), 7.12 (dd, J=8.8, 2.1 Hz, 1H), 5.09 (dd, J=12.8, 5.3 Hz, 1H), 4.51 (d, J=10.6 Hz, 3H), 3.97 (d, J=12.1 Hz, 2H), 3.92-3.78 (m, 3H), 3.17 (t, J=11.6 Hz, 2H), 3.02-2.89 (m, 2H), 2.57 (dd, J=19.0, 10.4 Hz, 2H), 2.37 (d, J=9.9 Hz, 1H), 2.21 (d, J=2.4 Hz, 2H), 2.15-2.00 (m, 5H), 1.92 (dd, J=21.8, 8.9 Hz, 4H), 1.64 (dd, J=23.4, 11.7 Hz, 2H), 1.57-1.46 (m, 2H), 1.34 (dt, J=15.0, 9.1 Hz, 2H). LCMS (ESI) calcd for C₄₃H₄₇ClN₉O₆⁺ [M+H]⁺: 820.33, found, 820.30.

### Example 39: Preparation of N-((1r,4r)-4-(3-chloro-4-cyanophenoxy)cyclohexyl)-6-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)pyridazine-3-carboxamide (GT-03490)

Referring to the method of Scheme 4, the target compound (GT-03490) was prepared, which is a yellow solid (49 mg, yield 56.79%). ¹H NMR (400 MHz, DMSO) δ 11.09 (d, J=5.5 Hz, 1H), 8.59 (t, J=8.3 Hz, 1H), 7.84 (d, J=8.6 Hz, 2H), 7.77 (dd, J=8.3, 4.9 Hz, 1H), 7.38 (t, J=7.3 Hz, 2H), 7.20-7.07 (m, 3H), 5.12-5.05 (m, 1H), 4.64 (s, 1H), 4.51 (d, J=7.2 Hz, 3H), 3.98 (dd, J=27.3, 14.1 Hz, 2H), 3.85 (dd, J=7.1, 3.4 Hz, 1H), 3.48-3.31 (m, 2H), 3.16 (dd, J=10.1, 6.4 Hz, 1H), 3.02 (dd, J=28.0, 13.7 Hz, 2H), 2.82 (d, J=17.7 Hz, 2H), 2.59 (d, J=19.0 Hz, 2H), 2.08 (t, J=10.4 Hz, 3H), 2.03-1.98 (m, 1H), 1.88 (d, J=3.9 Hz, 4H), 1.75-1.42 (m, 6H), 1.27 (ddd, J=32.5, 18.8, 10.9 Hz, 3H). LCMS (ESI) calcd for C₄₂H₄₅ClN₉O₆⁺ [M+H]⁺: 806.31, found, 806.40.

### Example 40: Preparation of N-((1r,3r)-3-(3-chloro-4-cyanophenoxy)-2,2,4,4-tetramethylcyclobutyl)-6-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)nicotinamide (GT-03494)

Referring to the method of Scheme 4, the target compound (GT-03494) was prepared, which is a yellow solid (40 mg, yield 45.84%). ¹H NMR (400 MHz, DMSO) δ 11.11 (s, 1H), 8.58 (s, 1H), 8.16 (d, J=8.4 Hz, 1H), 7.90 (d, J=8.7 Hz, 2H), 7.72 (dd, J=12.4, 1.8 Hz, 1H), 7.35 (dd, J=7.5, 3.4 Hz, 1H), 7.21 (d, J=2.4 Hz, 2H), 7.01 (dd, J=8.8, 2.4 Hz, 1H), 5.10 (dd, J=12.9, 5.2 Hz, 1H), 4.45 (d, J=13.7 Hz, 2H), 4.33 (d, J=11.9 Hz, 2H), 4.07 (d, J=9.2 Hz, 1H), 3.90 (s, 1H), 3.74 (d, J=13.0 Hz, 2H), 3.60 (d, J=13.7 Hz, 2H), 3.24-3.18 (m, 2H), 3.10 (t, J=13.2 Hz, 2H), 2.88 (td, J=14.3, 7.4 Hz, 1H), 2.64-2.52 (m, 2H), 2.32-2.14 (m, 2H), 2.15-1.95 (m, 5H), 1.92-1.84 (m, 1H), 1.37-1.25 (m, 2H), 1.23 (s, 6H), 1.12 (s, 6H). LCMS (ESI) calcd for C₄₆H₅₃ClFN₈O₆⁺ [M+H]⁺: 867.37, found, 867.40.

### Example 41: Preparation of N-((1r,4r)-4-(3-chloro-4-cyanophenoxy)cyclohexyl)-6-(4-((4-(2-(2,6-dioxopiperidin-3-yl)-1-oxoisoindolin-5-yl)piperidin-1-yl)methyl)piperidin-1-yl)pyridazine-3-carboxamide (GT-03518)

Referring to the method of Scheme 4, the target compound (GT-03518) was prepared, which is a yellow solid (49.00 mg, yield 58.84%). ¹H NMR (400 MHz, DMSO) δ 10.99 (s, 1H), 10.64 (s, 1H), 8.61 (d, J=8.2 Hz, 1H), 7.86 (dd, J=9.2, 7.2 Hz, 2H), 7.71 (d, J=7.9 Hz, 1H), 7.50 (d, J=6.3 Hz, 2H), 7.40 (dd, J=18.0, 5.2 Hz, 2H), 7.13 (dd, J=8.8, 2.4 Hz, 1H), 5.11 (dd, J=13.2, 5.1 Hz, 1H), 4.58-4.41 (m, 4H), 4.32 (d, J=17.5 Hz, 1H), 3.62 (d, J=11.3 Hz, 2H), 3.20-2.85 (m, 8H), 2.60 (d, J=16.7 Hz, 1H), 2.44-2.28 (m, 3H), 2.13-1.86 (m, 9H), 1.67-1.48 (m, 4H), 1.28 (dd, J=19.6, 9.5 Hz, 2H). LCMS (ESI) calcd for C₄₂H₄₈ClN₈O₅⁺ [M+H]⁺: 779.34, found, 779.30.

### Example 42: Preparation of N-((1r,4r)-4-(3-chloro-4-cyanophenoxy)cyclohexyl)-6-(4-(((3S,4S)-4-(2-(2,6-dioxopiperidin-3-yl)-1-oxoisoindolin-5-yl)-3,4-dihydroxypiperidin-1-yl)methyl)piperidin-1-yl)pyridazine-3-carboxamide (GT-03519)

Referring to the method of Scheme 4, the target compound (GT-03519) was prepared, which is a yellow solid (39.00 mg, yield 44.99%). ¹H NMR (400 MHz, DMSO) δ 10.99 (s, 1H), 10.88 (s, 1H), 8.61 (d, J=8.2 Hz, 1H), 7.90-7.83 (m, 2H), 7.74 (d, J=8.0 Hz, 1H), 7.65 (dd, J=13.6, 5.4 Hz, 1H), 7.47 (d, J=9.7 Hz, 1H), 7.39 (d, J=2.4 Hz, 1H), 7.13 (dd, J=8.8, 2.4 Hz, 1H), 5.12 (dd, J=13.2, 4.9 Hz, 1H), 4.63-4.44 (m, 5H), 4.37 (d, J=11.5 Hz, 1H), 3.38 (dd, J=36.3, 11.4 Hz, 2H), 3.19-3.03 (m, 6H), 2.94 (ddd, J=17.8, 16.6, 9.6 Hz, 1H), 2.67-2.58 (m, 1H), 2.41-2.22 (m, 2H), 2.16-1.97 (m, 5H), 1.90 (d, J=10.8 Hz, 2H), 1.79 (d, J=13.2 Hz, 1H), 1.72-1.39 (m, 5H), 1.29 (dt, J=20.3, 9.0 Hz, 3H). LCMS (ESI) calcd for C₄₂H₄₈ClN₈O₇⁺ [M+H]⁺: 811.33, found, 811.30.

### Example 43: Preparation of N-((1r,4r)-4-(3-chloro-4-cyanophenoxy)cyclohexyl)-6-(4-((4-(2-(2,6-dioxopiperidin-3-yl)-1-oxoisoindolin-5-yl)-3,6-dihydropyridin-1(2H)-yl)methyl)piperidin-1-yl)pyridazine-3-carboxamide (GT-03520)

Referring to the method of Scheme 4, the target compound (GT-03520) was prepared, which is a yellow solid (46.00 mg, yield 55.38%). ¹H NMR (400 MHz, DMSO) δ 11.01 (s, 1H), 10.78 (s, 1H), 8.62 (d, J=8.2 Hz, 1H), 7.86 (dd, J=9.2, 5.9 Hz, 2H), 7.74 (d, J=8.3 Hz, 2H), 7.65 (d, J=8.0 Hz, 1H), 7.49 (d, J=9.7 Hz, 1H), 7.38 (d, J=2.4 Hz, 1H), 7.13 (dd, J=8.8, 2.4 Hz, 1H), 6.33 (s, 1H), 5.13 (dd, J=13.2, 5.1 Hz, 1H), 4.60-4.43 (m, 4H), 4.35 (d, J=17.6 Hz, 1H), 4.16-4.06 (m, 1H), 3.69-3.62 (m, 1H), 3.30 (dd, J=16.3, 8.3 Hz, 1H), 3.20-3.02 (m, 5H), 2.97-2.87 (m, 1H), 2.77 (dd, J=17.9, 10.2 Hz, 1H), 2.60 (d, J=14.6 Hz, 1H), 2.46-2.26 (m, 2H), 2.13-1.85 (m, 7H), 1.67-1.48 (m, 4H), 1.31 (dd, J=14.5, 7.8 Hz, 2H). LCMS (ESI) calcd for C₄₂H₄₆ClN₈O₅⁺ [M+H]⁺: 777.32, found, 777.30.

### Example 44: Preparation of N-((1r,4r)-4-(3-chloro-4-cyanophenoxy)cyclohexyl)-6-(4-((4-(2-(2,6-dioxopiperidin-3-yl)-4-fluoro-1-oxoisoindolin-5-yl)piperidin-1-yl)methyl)piperidin-1-yl)pyridazine-3-carboxamide (GT-03525)

Referring to the method of Scheme 4, the target compound (GT-03525) was prepared, which is a yellow solid (27 mg, yield 31.69%). ¹H NMR (400 MHz, DMSO) δ 11.02 (s, 1H), 8.62 (d, J=8.2 Hz, 1H), 7.87 (t, J=9.2 Hz, 2H), 7.64 (d, J=7.8 Hz, 1H), 7.51 (dd, J=8.2, 4.7 Hz, 2H), 7.39 (d, J=2.4 Hz, 1H), 7.14 (dd, J=8.8, 2.4 Hz, 1H), 5.13 (dd, J=13.2, 5.0 Hz, 1H), 4.61-4.48 (m, 4H), 4.40 (d, J=17.3 Hz, 1H), 3.87 (dd, J=7.7, 3.5 Hz, 1H), 3.64 (d, J=11.1 Hz, 2H), 3.36-3.27 (m, 1H), 3.13 (dd, J=24.8, 11.8 Hz, 4H), 3.04-2.88 (m, 3H), 2.61 (d, J=16.6 Hz, 1H), 2.47-2.40 (m, 2H), 2.35-2.24 (m, 1H), 2.15-2.00 (m, 5H), 1.91 (d, J=10.0 Hz, 4H), 1.59 (dq, J=23.0, 10.3 Hz, 5H), 1.32 (dd, J=14.4, 7.9 Hz, 2H). LCMS (ESI) calcd for C₄₂H₄₇ClFN₈O₅⁺ [M+H]⁺: 797.33, found, 797.30.

### Example 45: Preparation of N-((1r,4r)-4-(3-chloro-4-cyanophenoxy)cyclohexyl)-6-(4-((4-(2-(2,6-dioxopiperidin-3-yl)-4-fluoro-1-oxoisoindolin-5-yl)-3,6-dihydropyridin-1(2H)-yl)methyl)piperidin-1-yl)pyridazine-3-carboxamide (GT-03540)

Referring to the method of Scheme 4, the target compound (GT-03540) was prepared, which is a yellow solid (53 mg, yield 62.37%). ¹H NMR (400 MHz, DMSO) δ 11.02 (s, 1H), 8.61 (d, J=8.2 Hz, 1H), 7.86 (t, J=8.8 Hz, 2H), 7.60 (dd, J=17.2, 7.2 Hz, 2H), 7.50 (d, J=9.7 Hz, 1H), 7.38 (d, J=2.3 Hz, 1H), 7.13 (dd, J=8.8, 2.3 Hz, 1H), 6.13 (s, 1H), 5.13 (dd, J=13.2, 5.0 Hz, 1H), 4.62-4.48 (m, 4H), 4.41 (d, J=17.5 Hz, 1H), 3.97-3.75 (m, 3H), 3.64 (d, J=11.5 Hz, 1H), 3.34-3.23 (m, 1H), 3.11 (t, J=11.5 Hz, 5H), 2.97-2.87 (m, 1H), 2.64 (dd, J=26.9, 17.4 Hz, 2H), 2.48-2.37 (m, 1H), 2.29 (dt, J=14.9, 7.7 Hz, 1H), 2.18-1.96 (m, 5H), 1.90 (d, J=10.1 Hz, 2H), 1.64 (dd, J=23.6, 10.9 Hz, 2H), 1.52 (t, J=11.3 Hz, 2H), 1.28 (dt, J=24.3, 11.7 Hz, 2H). LCMS (ESI) calcd for C₄₂H₄₅ClFN₈O₅⁺ [M+H]⁺: 795.31, found, 795.30.

### Example 46: Preparation of N-((1r,4r)-4-(3-chloro-4-cyanophenoxy)cyclohexyl)-6-(4-((4-(2-(2,6-dioxopiperidin-3-yl)-7-fluoro-1-oxoisoindolin-5-yl)piperidin-1-yl)methyl)piperidin-1-yl)pyridazine-3-carboxamide (GT-03580)

Referring to the method of Scheme 4, the target compound (GT-03580) was prepared, which is a yellow solid (50 mg, yield 58.61%). ¹H NMR (400 MHz, DMSO) δ 11.00 (s, 1H), 8.61 (d, J=8.1 Hz, 1H), 7.85 (d, J=8.6 Hz, 2H), 7.47 (d, J=9.4 Hz, 1H), 7.40-7.30 (m, 2H), 7.24-7.06 (m, 2H), 5.08 (dd, J=13.3, 5.0 Hz, 1H), 4.56-4.45 (m, 4H), 4.34 (d, J=17.9 Hz, 1H), 3.62 (d, J=10.9 Hz, 2H), 3.18-2.97 (m, 7H), 2.94-2.84 (m, 1H), 2.60 (d, J=16.0 Hz, 1H), 2.47-2.20 (m, 4H), 2.10 (d, J=9.5 Hz, 2H), 2.06-1.94 (m, 5H), 1.90 (d, J=10.2 Hz, 2H), 1.64 (dd, J=23.9, 11.3 Hz, 2H), 1.55-1.43 (m, 2H), 1.40-1.10 (m, 3H). LCMS (ESI) calcd for C₄₂H₄₇ClFN₈O₅⁺ [M+H]⁺: 797.33, found, 797.30.

### Example 47: Preparation of N-((1r,3r)-3-(3-chloro-4-cyanophenoxy)-2,2,4,4-tetramethylcyclobutyl)-6-(4-((4-(2-(2,6-dioxopiperidin-3-yl)-1-oxoisoindolin-5-yl)piperidin-1-yl)methyl)piperidin-1-yl)nicotinamide (GT-03589)

Referring to the method of Scheme 4, the target compound (GT-03589) was prepared, which is a yellow solid (53 mg, yield 65.07%). ¹H NMR (400 MHz, DMSO) δ 10.99 (s, 1H), 8.59 (d, J=1.6 Hz, 1H), 8.16 (d, J=8.4 Hz, 1H), 7.91 (d, J=8.7 Hz, 2H), 7.72 (d, J=7.9 Hz, 1H), 7.49 (s, 1H), 7.43 (d, J=8.0 Hz, 1H), 7.21 (d, J=2.3 Hz, 2H), 7.01 (dd, J=8.8, 2.3 Hz, 1H), 5.11 (dd, J=13.2, 5.0 Hz, 1H), 4.46 (d, J=17.0 Hz, 3H), 4.32 (d, J=17.2 Hz, 2H), 4.07 (d, J=9.1 Hz, 1H), 3.63 (d, J=11.6 Hz, 2H), 3.00 (ddt, J=22.7, 18.1, 8.5 Hz, 8H), 2.60 (d, J=16.8 Hz, 1H), 2.37 (ddd, J=37.5, 19.9, 11.1 Hz, 4H), 2.00 (t, J=14.4 Hz, 5H), 1.32-1.22 (m, 8H), 1.13 (s, 6H). LCMS (ESI) calcd for C₄₅H₅₃ClN₇O₅⁺ [M+H]⁺: 806.37, found, 806.40.

### Example 48: Preparation of N-((1r,3r)-3-(3-chloro-4-cyanophenoxy)-2,2,4,4-tetramethylcyclobutyl)-6-(4-((4-(2-(2,6-dioxopiperidin-3-yl)-1-oxoisoindolin-5-yl)-3,6-dihydropyridin-1(2H)-yl)methyl)piperidin-1-yl)nicotinamide (GT-03590)

Referring to the method of Scheme 4, the target compound (GT-03590) was prepared, which is a yellow solid (45 mg, yield 55.38%). ¹H NMR (400 MHz, DMSO) δ 11.00 (s, 1H), 10.67 (s, 1H), 8.59 (d, J=2.1 Hz, 1H), 8.19 (d, J=11.0 Hz, 1H), 7.91 (d, J=8.7 Hz, 2H), 7.78-7.69 (m, 2H), 7.66 (d, J=8.0 Hz, 1H), 7.21 (d, J=2.4 Hz, 2H), 7.02-6.99 (m, 1H), 6.34 (s, 1H), 5.13 (dd, J=13.2, 5.1 Hz, 1H), 4.65-4.54 (m, 1H), 4.50-4.43 (m, 3H), 4.38-4.33 (m, 2H), 4.07 (d, J=9.2 Hz, 2H), 3.82 (dd, J=15.9, 5.1 Hz, 2H), 3.71-3.63 (m, 2H), 3.34-3.27 (m, 1H), 3.14 (s, 2H), 3.02 (d, J=8.6 Hz, 1H), 2.91 (dd, J=10.6, 6.8 Hz, 1H), 2.78 (dd, J=19.9, 11.1 Hz, 1H), 2.64-2.58 (m, 1H), 2.47-2.32 (m, 2H), 2.05-1.98 (m, 2H), 1.26 (d, J=22.0 Hz, 8H), 1.13 (s, 6H). LCMS (ESI) calcd for C₄₅H₅₁ClN₇O₅⁺ [M+H]⁺: 804.36, found, 804.40.

### Example 49: Preparation of N-((1r,4r)-4-(3-chloro-4-cyanophenoxy)cyclohexyl)-6-(4-((8-(2-(2,6-dioxopiperidin-3-yl)-1-oxoisoindolin-5-yl)-3,8-diazabicyclo[3.2.1]octan-3-yl)methyl)piperidin-1-yl)pyridazine-3-carboxamide (GT-03772)

Referring to the method of Scheme 4, the target compound (GT-03772) was prepared, which is a yellow solid (33 mg, yield 38.30%). ¹H NMR (400 MHz, DMSO) δ 10.94 (s, 1H), 10.48 (s, 1H), 8.57 (d, J=8.2 Hz, 1H), 7.84 (dd, J=9.2, 3.4 Hz, 2H), 7.58 (d, J=8.4 Hz, 1H), 7.43 (d, J=9.7 Hz, 1H), 7.38 (d, J=2.4 Hz, 1H), 7.14-7.01 (m, 3H), 5.06 (dd, J=13.2, 5.1 Hz, 1H), 4.64-4.50 (m, 3H), 4.41 (d, J=12.9 Hz, 2H), 4.34 (d, J=17.0 Hz, 1H), 4.23 (d, J=17.0 Hz, 1H), 3.41 (d, J=10.6 Hz, 2H), 3.03 (t, J=11.3 Hz, 4H), 2.95-2.83 (m, 3H), 2.59 (d, J=17.3 Hz, 1H), 2.46-2.33 (m, 3H), 2.22 (s, 1H), 2.08 (t, J=11.9 Hz, 4H), 1.88 (dd, J=7.3, 4.3 Hz, 4H), 1.70-1.44 (m, 5H), 1.37-1.11 (m, 3H). LCMS (ESI) calcd for C₄₃H₄₉ClN₉O₅⁺ [M+H]⁺: 806.35, found, 806.40.

### Example 50: Preparation of N-((1r,4r)-4-(3-chloro-4-cyanophenoxy)cyclohexyl)-6-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)pyridazine-3-carboxamide (GT-03773)

Referring to the method of Scheme 4, the target compound (GT-03773) was prepared, which is a yellow solid (49 mg, yield 54.71%). ¹H NMR (400 MHz, DMSO) δ 11.09 (s, 1H), 8.59 (d, J=8.2 Hz, 1H), 7.85 (d, J=8.8 Hz, 2H), 7.71 (dd, J=12.4, 2.1 Hz, 1H), 7.46 (d, J=9.5 Hz, 1H), 7.40-7.31 (m, 2H), 7.13 (dd, J=8.8, 2.4 Hz, 1H), 5.09 (dd, J=12.9, 5.3 Hz, 1H), 4.56-4.44 (m, 3H), 4.31 (s, 1H), 3.95-3.79 (m, 4H), 3.67 (d, J=42.8 Hz, 1H), 3.29-3.13 (m, 3H), 3.06 (t, J=12.1 Hz, 2H), 2.95-2.82 (m, 1H), 2.62-2.54 (m, 2H), 2.28-2.21 (m, 1H), 2.04 (ddd, J=24.0, 18.7, 11.4 Hz, 7H), 1.93-1.84 (m, 3H), 1.76-1.42 (m, 5H), 1.34-1.24 (m, 2H). LCMS (ESI) calcd for C₄₃H₄₆ClFN₉O₆⁺ [M+H]⁺: 838.32, found, 838.40.

### Example 51: Preparation of N-((1r,4r)-4-(3-chloro-4-cyanophenoxy)cyclohexyl)-6-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)pyridazine-3-carboxamide (GT-03885)

Referring to the method of Scheme 4, the target compound (GT-03885) was prepared, which is a yellow solid (52.00 mg, yield 59.32%). LCMS (ESI) calcd for C₄₃H₄₇ClN₉O₆⁺ [M+H]⁺: 820.33, found, 820.30.

### Example 52: Preparation of N-((1r,4r)-4-(3-chloro-4-cyanophenoxy)cyclohexyl)-6-(4-((3-(2-(2,6-dioxopiperidin-3-yl)-1-oxoisoindolin-5-yl)-3,8-diazabicyclo[3.2.1]octan-8-yl)methyl)piperidin-1-yl)pyridazine-3-carboxamide (GT-03886)

Referring to the method of Scheme 4, the target compound (GT-03886) was prepared, which is a yellow solid (47.00 mg, yield 54.55%). LCMS (ESI) calcd for C₄₃H₄₉ClN₉O₅⁺ [M+H]⁺: 806.35, found, 806.30.

### Example 53: Preparation of N-(2-chloro-6-methylphenyl)-2-((6-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)-2-methylpyrimidin-4-yl)amino)thiazole-5-carboxamide (GT-03627)

Referring to the method of Scheme 4, the target compound (GT-03627) was prepared, which is a yellow solid (64 mg, yield 71.65%). ¹H NMR (400 MHz, DMSO) δ 11.11 (s, 1H), 10.40 (s, 1H), 9.99 (s, 1H), 8.28 (s, 1H), 7.79 (d, J=12.1 Hz, 1H), 7.57 (d, J=7.5 Hz, 1H), 7.40 (d, J=6.1 Hz, 1H), 7.28 (dd, J=10.4, 6.9 Hz, 2H), 6.21 (s, 1H), 5.11 (dd, J=12.8, 5.3 Hz, 1H), 4.68 (s, 2H), 3.48 (d, J=10.2 Hz, 2H), 3.18 (dd, J=16.5, 9.0 Hz, 2H), 3.09-2.77 (m, 6H), 2.57 (dd, J=21.9, 11.3 Hz, 2H), 2.47 (s, 3H), 2.39 (d, J=7.6 Hz, 2H), 2.24 (s, 3H), 2.20 (s, 1H), 2.10-1.99 (m, 3H), 1.88 (d, J=11.3 Hz, 2H), 1.22 (q, J=13.0 Hz, 3H). LCMS (ESI) calcd for C₄₁H₄₃ClFN₁₀O₅⁺ [M+H]⁺: 842.36, found, 842.30.

### Example 54: Preparation of N-(2-chloro-6-methylphenyl)-2-((6-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)-2-methylpyrimidin-4-yl)amino)thiazole-5-carboxamide (GT-03628)

Referring to the method of Scheme 4, the target compound (GT-03628) was prepared, which is a yellow solid (52 mg, yield 59.21%). ¹H NMR (400 MHz, DMSO) δ 11.28 (s, 1H), 11.11 (s, 1H), 10.04 (s, 1H), 8.31 (s, 1H), 7.76 (d, J=10.9 Hz, 1H), 7.39 (dd, J=7.3, 1.1 Hz, 1H), 7.34-7.23 (m, 3H), 6.26 (s, 1H), 5.11 (dd, J=12.7, 5.4 Hz, 1H), 4.65 (s, 2H), 4.18 (s, 3H), 3.84 (d, J=8.8 Hz, 3H), 3.42 (d, J=30.1 Hz, 2H), 2.97-2.79 (m, 6H), 2.59 (d, J=19.3 Hz, 1H), 2.49 (d, J=1.7 Hz, 3H), 2.24 (s, 3H), 2.20-2.08 (m, 1H), 2.04-1.98 (m, 1H), 1.93 (d, J=11.6 Hz, 2H), 1.19 (dd, J=20.5, 9.8 Hz, 2H). LCMS (ESI) calcd for C₄₀H₄₁ClFN₁₀O₅⁺ [M+H]⁺: 828.33, found, 828.30.

### Example 55: Preparation of N-(2-chloro-6-methylphenyl)-2-((6-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)-2-methylpyrimidin-4-yl)amino)thiazole-5-carboxamide (GT-03629)

Referring to the method of Scheme 4, the target compound (GT-03629) was prepared, which is a yellow solid (46 mg, yield 52.63%). ¹H NMR (400 MHz, DMSO) δ 11.09 (s, 1H), 10.55 (s, 1H), 10.07 (s, 1H), 8.32 (s, 1H), 7.75 (d, J=8.4 Hz, 1H), 7.39 (dd, J=4.8, 1.9 Hz, 2H), 7.31-7.23 (m, 3H), 6.29 (s, 1H), 5.10 (dd, J=12.9, 5.4 Hz, 1H), 4.77 (s, 2H), 4.19 (d, J=9.2 Hz, 3H), 3.42 (d, J=10.3 Hz, 2H), 3.10-2.97 (m, 4H), 2.88 (dd, J=22.0, 8.6 Hz, 3H), 2.63-2.54 (m, 2H), 2.50 (s, 3H), 2.44 (d, J=7.6 Hz, 2H), 2.24 (s, 3H), 2.12-1.98 (m, 3H), 1.88 (d, J=11.4 Hz, 2H), 1.22 (dd, J=21.7, 11.5 Hz, 2H). LCMS (ESI) calcd for C₄₁H₄₄ClN₁₀O₅⁺ [M+H]⁺: 824.37, found, 824.30.

### Example 56: Preparation of N-(2-chloro-6-methylphenyl)-2-((6-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)-2-methylpyrimidin-4-yl)amino)thiazole-5-carboxamide (GT-03630)

Referring to the method of Scheme 4, the target compound (GT-03630) was prepared, which is a yellow solid (57 mg, yield 66.34%). ¹H NMR (400 MHz, DMSO) δ 11.26 (s, 1H), 11.09 (s, 1H), 10.05 (s, 1H), 8.31 (s, 1H), 7.77 (d, J=8.2 Hz, 1H), 7.42-7.37 (m, 1H), 7.30-7.23 (m, 2H), 7.16 (d, J=1.1 Hz, 1H), 6.95 (dd, J=8.3, 1.6 Hz, 1H), 6.26 (s, 1H), 5.10 (dd, J=12.8, 5.3 Hz, 1H), 4.65 (d, J=3.3 Hz, 2H), 4.21-4.15 (m, 2H), 3.76 (d, J=7.8 Hz, 2H), 3.39 (dt, J=11.2, 5.4 Hz, 2H), 2.92 (dd, J=22.4, 8.0 Hz, 3H), 2.86-2.71 (m, 4H), 2.67-2.53 (m, 2H), 2.48 (s, 3H), 2.23 (s, 3H), 2.11 (dd, J=11.3, 4.3 Hz, 1H), 2.04-1.98 (m, 1H), 1.91 (d, J=11.0 Hz, 2H), 1.18 (dd, J=21.9, 11.1 Hz, 2H).

LCMS (ESI) calcd for C₄₀H₄₂ClN₁₀O₅⁺ [M+H]⁺: 810.34, found, 810.30.

### Example 57: Preparation of N-(2-chloro-6-methylphenyl)-2-((6-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)-2-methylpyrimidin-4-yl)amino)thiazole-5-carboxamide (GT-03631)

Referring to the method of Scheme 4, the target compound (GT-03631) was prepared, which is a yellow solid (55 mg, yield 64.01%). ¹H NMR (400 MHz, DMSO) δ 11.52 (s, 1H), 11.09 (d, J=6.1 Hz, 1H), 10.05 (s, 1H), 8.31 (s, 1H), 7.78 (dd, J=8.3, 4.7 Hz, 1H), 7.39 (d, J=7.3 Hz, 1H), 7.28 (dd, J=9.6, 7.4 Hz, 2H), 7.18 (s, 1H), 7.10 (d, J=8.5 Hz, 1H), 6.28 (d, J=16.3 Hz, 1H), 5.13-5.06 (m, 1H), 4.64 (s, 1H), 4.49 (d, J=6.1 Hz, 1H), 4.35-4.20 (m, 3H), 3.44-3.35 (m, 1H), 3.21-3.05 (m, 1H), 3.02-2.77 (m, 5H), 2.59 (d, J=19.0 Hz, 2H), 2.48 (s, 3H), 2.24 (s, 3H), 2.21-2.10 (m, 1H), 2.09-1.79 (m, 5H), 1.52-0.88 (m, 3H). LCMS (ESI) calcd for C₄₀H₄₂ClN₁₀O₅⁺ [M+H]⁺: 810.34, found, 810.30.

### Example 58: Preparation of N-(2-chloro-6-methylphenyl)-2-((6-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)-2-methylpyrimidin-4-yl)amino)thiazole-5-carboxamide (GT-03632)

Referring to the method of Scheme 4, the target compound (GT-03632) was prepared, which is a yellow solid (66 mg, yield 75.51%). ¹H NMR (400 MHz, DMSO) δ 11.62 (s, 1H), 11.09 (s, 1H), 9.96 (s, 1H), 8.27 (s, 1H), 7.76 (d, J=8.5 Hz, 1H), 7.38 (t, J=10.2 Hz, 2H), 7.27 (ddd, J=11.1, 7.2, 4.3 Hz, 3H), 6.20 (s, 1H), 5.09 (dd, J=12.9, 5.4 Hz, 1H), 4.35-4.24 (m, 2H), 4.18 (d, J=12.1 Hz, 3H), 3.86 (d, J=14.7 Hz, 1H), 3.76-3.68 (m, 2H), 3.07-2.82 (m, 5H), 2.62-2.56 (m, 1H), 2.46 (s, 3H), 2.34-2.14 (m, 7H), 2.04-1.93 (m, 5H), 1.27 (dd, J=21.7, 9.7 Hz, 2H). LCMS (ESI) calcd for C₄₁H₄₄ClN₁₀O₅⁺ [M+H]⁺: 824.37, found, 824.30.

### Example 59: Preparation of 7-cyclopentyl-2-((5-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)pyridin-2-yl)amino)-N,N-dimethyl-7H-pyrrolo[2,3-d]pyrimidine-6-carboxamide (GT-03651)

Referring to the method of Scheme 4, the target compound (GT-03651) was prepared, which is a yellow solid (73 mg, yield 81.14%). ¹H NMR (400 MHz, DMSO) δ 11.06 (s, 1H), 9.21 (s, 1H), 8.75 (s, 1H), 8.12 (d, J=9.1 Hz, 1H), 7.99 (d, J=2.6 Hz, 1H), 7.95 (s, 2H), 7.66 (d, J=8.5 Hz, 1H), 7.42 (dd, J=9.1, 2.6 Hz, 1H), 7.21 (s, 1H), 7.11 (d, J=8.2 Hz, 1H), 6.59 (s, 1H), 5.06 (dd, J=12.8, 5.4 Hz, 1H), 4.81-4.64 (m, 1H), 3.63 (d, J=10.7 Hz, 4H), 3.35 (s, 3H), 3.05 (s, 6H), 2.70-2.61 (m, 3H), 2.56 (dd, J=8.2, 5.4 Hz, 2H), 2.41 (ddd, J=9.6, 5.7, 2.4 Hz, 2H), 2.29 (d, J=6.7 Hz, 2H), 1.97 (tt, J=17.6, 10.9 Hz, 10H), 1.66-1.55 (m, 5H), 1.35-1.24 (m, 2H). LCMS (ESI) calcd for C₄₄H₅₂N₁₁O₅⁺ [M+H]⁺: 814.41, found, 814.40.

### Example 60: Preparation of 7-cyclopentyl-2-((5-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)pyridin-2-yl)amino)-N,N-dimethyl-7H-pyrrolo[2,3-d]pyrimidine-6-carboxamide (GT-03652)

Referring to the method of Scheme 4, the target compound (GT-03652) was prepared, which is a yellow solid (78.00 mg, yield 86.70%). ¹H NMR (400 MHz, DMSO) δ 11.06 (s, 1H), 9.21 (s, 1H), 8.75 (s, 1H), 8.12 (d, J=9.1 Hz, 1H), 7.99 (d, J=2.6 Hz, 1H), 7.95 (s, 2H), 7.66 (d, J=8.5 Hz, 1H), 7.42 (dd, J=9.1, 2.6 Hz, 1H), 7.21 (s, 1H), 7.11 (d, J=8.2 Hz, 1H), 6.59 (s, 1H), 5.06 (dd, J=12.8, 5.4 Hz, 1H), 4.79-4.66 (m, 1H), 3.63 (d, J=11.4 Hz, 4H), 3.35 (s, 2H), 3.05 (s, 8H), 2.69-2.54 (m, 5H), 2.42 (dd, J=7.8, 4.8 Hz, 2H), 2.29 (d, J=6.7 Hz, 2H), 2.01-1.86 (m, 9H), 1.61 (dt, J=12.6, 6.4 Hz, 5H), 1.34-1.23 (m, 2H). LCMS (ESI) calcd for C₄₄H₅₂N₁₁O₅⁺ [M+H]⁺: 814.41, found, 814.40.

### Example 61: Preparation of 7-cyclopentyl-2-((5-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)pyridin-2-yl)amino)-N,N-dimethyl-7H-pyrrolo[2,3-d]pyrimidine-6-carboxamide (GT-03654)

Referring to the method of Scheme 4, the target compound (GT-03654) was prepared, which is a yellow solid (75 mg, yield 82.96%). ¹H NMR (400 MHz, DMSO) δ 11.48 (s, 2H), 11.11 (s, 1H), 8.98 (s, 1H), 8.11 (dd, J=9.5, 2.3 Hz, 1H), 7.90 (s, 1H), 7.77 (d, J=10.9 Hz, 1H), 7.61 (d, J=9.4 Hz, 1H), 7.33 (d, J=7.3 Hz, 1H), 6.81 (s, 1H), 5.11 (dd, J=12.8, 5.4 Hz, 1H), 4.81 (dd, J=17.3, 8.7 Hz, 1H), 4.65 (s, 2H), 3.84 (d, J=9.5 Hz, 2H), 3.64 (d, J=12.1 Hz, 3H), 3.05 (s, 6H), 2.85 (dd, J=14.1, 8.8 Hz, 5H), 2.71 (t, J=11.5 Hz, 2H), 2.64-2.51 (m, 2H), 2.30 (ddd, J=8.6, 6.0, 3.3 Hz, 2H), 1.98 (ddd, J=24.5, 16.8, 11.7 Hz, 9H), 1.68-1.61 (m, 2H), 1.39-1.25 (m, 2H). LCMS (ESI) calcd for C₄₃H₄₈FN₁₁O₅⁺ [M+H]⁺: 818.38, found, 818.40.

### Example 62: Preparation of 7-cyclopentyl-2-((5-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)pyridin-2-yl)amino)-N,N-dimethyl-7H-pyrrolo[2,3-d]pyrimidine-6-carboxamide (GT-03655)

Referring to the method of Scheme 4, the target compound (GT-03655) was prepared, which is a yellow solid (75 mg, yield 81.56%). ¹H NMR (400 MHz, DMSO) δ 11.50 (s, 1H), 11.11 (s, 1H), 8.99 (s, 1H), 8.13 (d, J=9.7 Hz, 1H), 7.92 (s, 1H), 7.79 (d, J=12.1 Hz, 1H), 7.58 (t, J=9.7 Hz, 2H), 6.82 (s, 1H), 5.12 (dd, J=12.9, 5.4 Hz, 1H), 4.82 (dd, J=17.3, 8.6 Hz, 1H), 4.68 (s, 2H), 3.64 (d, J=12.2 Hz, 3H), 3.48 (d, J=10.5 Hz, 2H), 3.22-3.12 (m, 2H), 3.06 (s, 6H), 2.91 (ddd, J=15.6, 13.7, 11.3 Hz, 3H), 2.76 (dd, J=18.3, 7.4 Hz, 2H), 2.66-2.52 (m, 2H), 2.48-2.40 (m, 2H), 2.31 (ddd, J=8.3, 4.2, 1.0 Hz, 2H), 2.11-1.94 (m, 9H), 1.65 (dd, J=11.2, 5.3 Hz, 2H), 1.44-1.31 (m, 2H).

LCMS (ESI) calcd for C₄₄H₅₁FN₁₁O₅⁺ [M+H]⁺: 832.40, found, 832.40.

### Example 63: Preparation of 7-cyclopentyl-2-((5-(4-((4-(2-(2,6-dioxopiperidin-3-yl)-6-fluoro-1,3-dioxoisoindolin-5-yl)piperazin-1-yl)methyl)piperidin-1-yl)pyridin-2-yl)amino)-N,N-dimethyl-7H-pyrrolo[2,3-d]pyrimidine-6-carboxamide (GT-03656)

Referring to the method of Scheme 4, the target compound (GT-03656) was prepared, which is a yellow solid (81 mg, yield 92.78%). ¹H NMR (400 MHz, DMSO) δ 11.48 (s, 1H), 11.17 (s, 1H), 11.12 (s, 1H), 9.00 (s, 1H), 8.15 (d, J=7.5 Hz, 1H), 7.94 (s, 1H), 7.82 (d, J=11.2 Hz, 1H), 7.61 (dd, J=17.2, 8.4 Hz, 2H), 6.82 (s, 1H), 5.13 (dd, J=12.8, 5.4 Hz, 1H), 4.80 (dd, J=17.5, 8.8 Hz, 1H), 3.75-3.63 (m, 6H), 3.23 (dd, J=18.8, 8.5 Hz, 3H), 3.12 (s, 2H), 3.06 (s, 6H), 2.95-2.79 (m, 3H), 2.65-2.52 (m, 2H), 2.35-2.27 (m, 2H), 2.14-1.92 (m, 9H), 1.70-1.62 (m, 2H), 1.41 (dd, J=20.5, 10.1 Hz, 2H). LCMS (ESI) calcd for C₄₂H₄₉FN₁₁O₅⁺ [M+H]⁺: 806.38, found, 806.40.

### Example 64: Preparation of 7-cyclopentyl-2-((5-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)pyridin-2-yl)amino)-N,N-dimethyl-7H-pyrrolo[2,3-d]pyrimidine-6-carboxamide (GT-03657)

Referring to the method of Scheme 4, the target compound (GT-03657) was prepared, which is a yellow solid (79 mg, yield 84.73%). ¹H NMR (400 MHz, DMSO) δ 11.40 (s, 1H), 11.10 (d, J=5.1 Hz, 1H), 8.98 (d, J=2.9 Hz, 1H), 8.11 (dd, J=14.2, 6.1 Hz, 1H), 7.93 (d, J=10.4 Hz, 1H), 7.81-7.71 (m, 1H), 7.63 (dd, J=7.6, 3.1 Hz, 1H), 7.36 (t, J=7.8 Hz, 1H), 6.81 (s, 1H), 5.16-5.05 (m, 1H), 4.84-4.77 (m, 1H), 4.58 (s, 1H), 4.42 (dd, J=20.1, 9.4 Hz, 1H), 4.22-4.12 (m, 2H), 3.70 (t, J=11.0 Hz, 3H), 3.44-3.39 (m, 3H), 3.06 (s, 6H), 2.95-2.85 (m, 2H), 2.81-2.70 (m, 2H), 2.65-2.53 (m, 2H), 2.35-2.27 (m, 2H), 2.07-1.90 (m, 9H), 1.66 (dd, J=12.4, 7.0 Hz, 2H), 1.40 (dd, J=24.6, 13.2 Hz, 2H). LCMS (ESI) calcd for C₄₃H₄₉FN₁₁O₅⁺ [M+H]⁺: 818.38, found, 818.40.

### Example 65: Preparation of 7-cyclopentyl-2-((5-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)pyridin-2-yl)amino)-N,N-dimethyl-7H-pyrrolo[2,3-d]pyrimidine-6-carboxamide (GT-03658)

Referring to the method of Scheme 4, the target compound (GT-03658) was prepared, which is a yellow solid (27 mg, yield 29.36%). ¹H NMR (400 MHz, DMSO) δ 11.57 (s, 1H), 11.11 (s, 1H), 9.01 (s, 1H), 8.18 (d, J=9.7 Hz, 1H), 7.99 (s, 1H), 7.80 (d, J=11.5 Hz, 1H), 7.66 (s, 1H), 7.47 (d, J=7.2 Hz, 1H), 6.83 (s, 1H), 5.12 (dd, J=12.6, 5.4 Hz, 1H), 4.84-4.79 (m, 1H), 4.19 (s, 1H), 4.05 (d, J=12.4 Hz, 2H), 3.72 (d, J=12.1 Hz, 3H), 3.57 (d, J=11.1 Hz, 2H), 3.06 (s, 6H), 2.99 (dd, J=15.5, 10.9 Hz, 2H), 2.85 (dd, J=19.1, 9.6 Hz, 3H), 2.63-2.53 (m, 2H), 2.29 (ddd, J=22.1, 14.2, 6.3 Hz, 6H), 2.06-1.97 (m, 7H), 1.65 (d, J=5.9 Hz, 3H), 1.47 (dd, J=24.2, 13.2 Hz, 2H). LCMS (ESI) calcd for C₄₄H₅₁FN₁₁O₅⁺ [M+H]⁺: 832.40, found, 832.40.

### Example 66: Preparation of 7-cyclopentyl-2-((5-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)pyridin-2-yl)amino)-N,N-dimethyl-7H-pyrrolo[2,3-d]pyrimidine-6-carboxamide (GT-03659)

Referring to the method of Scheme 4, the target compound (GT-03659) was prepared, which is a yellow solid (82 mg, yield 7 94.63%). ¹H NMR (400 MHz, DMSO) δ 11.44 (s, 2H), 11.09 (s, 1H), 8.98 (s, 1H), 8.09 (d, J=9.5 Hz, 1H), 7.89 (s, 1H), 7.78 (d, J=8.2 Hz, 1H), 7.61 (d, J=9.2 Hz, 1H), 7.16 (s, 1H), 6.97 (d, J=8.2 Hz, 1H), 6.81 (s, 1H), 5.10 (dd, J=12.8, 5.4 Hz, 1H), 4.79 (dd, J=17.5, 8.8 Hz, 1H), 4.65 (d, J=4.5 Hz, 2H), 3.76 (d, J=8.3 Hz, 2H), 3.63 (d, J=11.9 Hz, 2H), 3.06 (s, 6H), 2.98-2.73 (m, 6H), 2.65 (dd, J=22.4, 10.5 Hz, 3H), 2.55 (d, J=11.4 Hz, 1H), 2.35-2.27 (m, 2H), 1.98 (ddd, J=19.9, 17.5, 13.3 Hz, 9H), 1.64 (dd, J=10.8, 4.9 Hz, 2H), 1.33 (dd, J=22.9, 11.9 Hz, 2H). LCMS (ESI) calcd for C₄₃H₅₀N₁₁O₅⁺ [M+H]⁺: 800.39, found, 800.50.

### Example 66: Preparation of 7-cyclopentyl-2-((5-(4-((4-(2-(2,6-dioxopiperidin-3-yl)-1,3-dioxoisoindolin-5-yl)piperazin-1-yl)methyl)piperidin-1-yl)pyridin-2-yl)amino)-N,N-dimethyl-7H-pyrrolo[2,3-d]pyrimidine-6-carboxamide (GT-03660)

Referring to the method of Scheme 4, the target compound (GT-03660) was prepared, which is a yellow solid (57 mg, yield 66.78%). ¹H NMR (400 MHz, DMSO) δ 11.53 (s, 1H), 11.22 (s, 1H), 11.09 (s, 1H), 9.00 (s, 1H), 8.16 (dd, J=9.6, 2.6 Hz, 1H), 7.94 (s, 1H), 7.76 (d, J=8.5 Hz, 1H), 7.62 (d, J=9.5 Hz, 1H), 7.49 (s, 1H), 7.36 (dd, J=8.7, 1.9 Hz, 1H), 6.82 (s, 1H), 5.10 (dd, J=12.9, 5.4 Hz, 1H), 4.81 (p, J=8.8 Hz, 1H), 4.19 (d, J=12.9 Hz, 2H), 3.73-3.65 (m, 5H), 3.19-3.08 (m, 4H), 3.06 (s, 6H), 2.86 (ddd, J=26.7, 13.2, 7.2 Hz, 3H), 2.65-2.52 (m, 2H), 2.35-2.27 (m, 2H), 2.20-1.86 (m, 9H), 1.68-1.60 (m, 2H), 1.40 (dd, J=20.7, 10.2 Hz, 2H). LCMS (ESI) calcd for C₄₂H₅₀N₁₁O₅⁺ [M+H]⁺: 788.39, found, 788.40.

### Example 67: Preparation of 7-cyclopentyl-2-((5-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)pyridin-2-yl)amino)-N,N-dimethyl-7H-pyrrolo[2,3-d]pyrimidine-6-carboxamide (GT-03661)

Referring to the method of Scheme 4, the target compound (GT-03660) was prepared, which is a yellow solid (57 mg, yield 64.47%). ¹H NMR (400 MHz, DMSO) δ 11.55 (s, 1H), 11.08 (s, 1H), 9.00 (d, J=4.0 Hz, 1H), 8.13 (dd, J=9.8, 2.8 Hz, 1H), 7.95 (d, J=18.1 Hz, 1H), 7.79 (dd, J=8.5, 3.6 Hz, 1H), 7.60 (d, J=9.4 Hz, 1H), 7.19 (dd, J=8.5, 1.6 Hz, 1H), 7.12 (dd, J=8.7, 2.2 Hz, 1H), 6.82 (d, J=1.8 Hz, 1H), 5.11-5.06 (m, 1H), 4.84-4.79 (m, 1H), 4.65 (s, 1H), 4.15 (d, J=12.2 Hz, 1H), 4.07-3.99 (m, 2H), 3.93 (d, J=13.3 Hz, 2H), 3.68 (d, J=12.4 Hz, 3H), 2.95-2.70 (m, 6H), 2.59 (d, J=19.1 Hz, 2H), 2.30 (dd, J=10.0, 7.3 Hz, 3H), 2.09-1.87 (m, 12H), 1.65 (d, J=4.8 Hz, 3H), 1.46-1.35 (m, 2H). LCMS (ESI) calcd for C₄₃H₅₀N₁₁O₅⁺ [M+H]⁺: 800.39, found, 800.40.

### Example 68: Preparation of 7-cyclopentyl-2-((5-(4-((4-((2-(2,6-dioxopiperidin-3-yl)-1-oxoisoindolin-5-yl)amino)piperidin-1-yl)methyl)piperidin-1-yl)pyridin-2-yl)amino)-N,N-dimethyl-7H-pyrrolo[2,3-d]pyrimidine-6-carboxamide (GT-03665)

Referring to the method of Scheme 4, the target compound (GT-03665) was prepared, which is a yellow solid (75 mg, yield 82.55%). ¹H NMR (400 MHz, DMSO) δ 11.63 (s, 1H), 10.93 (s, 1H), 9.02 (s, 1H), 8.20 (d, J=9.5 Hz, 1H), 8.06 (s, 1H), 7.67 (dd, J=9.5, 3.3 Hz, 1H), 7.46-7.34 (m, 1H), 6.83 (s, 1H), 6.75 (s, 1H), 5.02 (dd, J=13.2, 5.0 Hz, 1H), 4.84-4.77 (m, 1H), 4.29 (d, J=16.8 Hz, 2H), 4.16 (d, J=16.8 Hz, 2H), 3.88-3.78 (m, 1H), 3.69 (d, J=11.8 Hz, 2H), 3.58 (d, J=10.5 Hz, 2H), 3.41-3.33 (m, 1H), 3.05 (s, 7H), 3.00 (s, 1H), 2.93-2.82 (m, 3H), 2.58 (d, J=16.7 Hz, 1H), 2.36-2.26 (m, 3H), 1.99 (ddd, J=19.0, 17.8, 9.6 Hz, 12H), 1.69-1.59 (m, 2H), 1.46 (d, J=11.0 Hz, 2H). LCMS (ESI) calcd for C₄₃H₅₄N₁₁O₄⁺ [M+H]⁺: 789.34, found, 789.30.

### Example 69: Preparation of N-(2-chloro-6-methylphenyl)-2-((6-(4-((4-(2-(2,6-dioxopiperidin-3-yl)-1-oxoisoindolin-5-yl)piperidin-1-yl)methyl)piperidin-1-yl)-2-methylpyrimidin-4-yl)amino)thiazole-5-carboxamide (GT-03533)

Referring to the method of Scheme 4, the target compound (GT-03533) was prepared, which is a white solid (26 mg, yield 32%). ¹H NMR (300 MHz, DMSO) δ 11.45 (s, 1H), 11.02 (s, 1H), 9.91 (s, 1H), 8.22 (d, J=14.3 Hz, 2H), 7.68 (d, J=7.8 Hz, 1H), 7.53 (s, 1H), 7.43 (d, J=7.6 Hz, 2H), 7.38-7.22 (m, 2H), 6.10 (s, 1H), 5.14 (dd, J=13.2, 5.0 Hz, 1H), 4.39 (dd, J=42.6, 17.3 Hz, 4H), 3.05-2.86 (m, 6H), 2.63 (d, J=15.9 Hz, 2H), 2.43 (s, 4H), 2.27 (s, 6H), 2.13-1.97 (m, 4H), 1.83 (d, J=13.9 Hz, 6H). LCMS (ESI) calcd for C₄₀H₄₅ClN₉O₄⁺ [M+H]⁺: 782.3, found, 782.0.

### Example 70: Preparation of N-(2-chloro-6-methylphenyl)-2-((6-(4-(((3S,4S)-4-(2-(2,6-dioxopiperidin-3-yl)-1-oxoisoindolin-5-yl)-3,4-dihydroxypiperidin-1-yl)methyl)piperidin-1-yl)-2-methylpyrimidin-4-yl)amino)thiazole-5-carboxamide (GT-03534)

Referring to the method of Scheme 4, the target compound (GT-03534) was prepared, which is a white solid (4 mg, yield 4%). ¹H NMR (300 MHz, DMSO) δ 11.45 (s, 1H), 11.02 (s, 1H), 9.91 (s, 1H), 8.22 (d, J=19.0 Hz, 2H), 7.72 (d, J=17.8 Hz, 3H), 7.44 (d, J=6.8 Hz, 1H), 7.30 (d, J=7.5 Hz, 2H), 6.10 (s, 1H), 5.13 (s, 1H), 4.78 (s, 1H), 4.60-4.53 (m, 1H), 4.40 (dd, J=41.8, 17.2 Hz, 6H), 3.92 (s, 1H), 2.91 (s, 2H), 2.76 (s, 2H), 2.65 (s, 2H), 2.44 (s, 4H), 2.27 (s, 5H), 1.88 (s, 6H). LCMS (ESI) calcd for C₄₀H₄₅ClN₉O₆⁺ [M+H]⁺: 813.2, found, 814.2.

### Example 71: Preparation of N-(2-chloro-6-methylphenyl)-2-((6-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)-2-methylpyrimidin-4-yl)amino)thiazole-5-carboxamide (GT-03537)

Referring to the method of Scheme 4, the target compound (GT-03537) was prepared, which is a yellow solid (10 mg, yield 11.23%). ¹H NMR (400 MHz, DMSO) δ 11.39 (s, 1H), 11.09 (s, 1H), 9.86 (s, 1H), 8.21 (s, 1H), 7.68 (d, J=12.1 Hz, 1H), 7.43-7.18 (m, 4H), 6.07 (s, 1H), 5.09 (dd, J=12.8, 5.3 Hz, 1H), 4.28 (s, 2H), 3.35 (d, J=9.5 Hz, 2H), 3.27 (s, 3H), 3.10 (d, J=10.4 Hz, 2H), 2.88 (t, J=12.1 Hz, 3H), 2.59 (d, J=18.0 Hz, 1H), 2.40 (s, 3H), 2.23 (d, J=7.7 Hz, 5H), 2.02 (d, J=5.2 Hz, 1H), 1.89 (s, 4H), 1.71 (d, J=6.8 Hz, 3H), 1.09 (d, J=9.1 Hz, 2H). LCMS (ESI) calcd for C₄₁H₄₃ClFN₁₀O₅⁺ [M+H]⁺: 840.27, found, 841.2.

### Example 72: Preparation of N-(2-chloro-6-methylphenyl)-2-((6-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)-2-methylpyrimidin-4-yl)amino)thiazole-5-carboxamide (GT-03538)

Referring to the method of Scheme 4, the target compound (GT-03538) was prepared, which is a yellow solid (20 mg, yield 21.69%). ¹H NMR (400 MHz, DMSO) δ 11.36 (s, 1H), 11.08 (s, 1H), 9.85 (s, 1H), 8.20 (s, 1H), 7.62 (d, J=13.9 Hz, 1H), 7.39 (d, J=7.8 Hz, 1H), 7.27 (d, J=11.7 Hz, 3H), 6.04 (s, 1H), 5.32 (s, 1H), 5.08 (d, J=13.0 Hz, 1H), 4.22 (s, 2H), 3.86 (d, J=10.6 Hz, 2H), 3.62 (s, 4H), 2.83 (s, 2H), 2.55 (d, J=11.3 Hz, 1H), 2.36 (d, J=22.5 Hz, 5H), 2.25 (d, J=9.4 Hz, 5H), 2.02 (d, J=7.4 Hz, 2H), 1.78 (s, 2H), 1.60 (d, J=8.5 Hz, 2H), 1.48-1.42 (m, 1H). LCMS (ESI) calcd for C₄₀H₄₁ClFN₁₀O₅⁺ [M+H]⁺: 826.26, found, 827.5.

### Biological Activity Assay

### Reagents and Materials

| Reagents and materials | Suppliers or Source |
|---|---|
| Human prostate cancer cell line: | ATCC (American Type |
| DU-145 | Culture Collection) |
| Human prostate cancer cell line: LNcap | ATCC |
| Human ovarian cancer cell line: PC-3 | ATCC |
| dexamethasone-resistant multiple | ATCC |
| myeloma cell line: MM.1R | |
| Human diffuse large B-cell lymphoma | Shanghai Medicilon Inc. |
| cell line: WSU-DLCL2 | (test) |
| Human gastric cancer cell line: | Dalian Meilun Biotech Co., Ltd. |
| MGC80-3 | |
| Human colorectal cancer cell line: | ATCC |
| SW620 | |
| Human non-small cell lung cancer cell | ATCC |
| line: A549 | |
| Human T cell acute lymphoblastic | Dalian Meilun Biotech Co., Ltd. |
| leukemia cell line: CCRF-CEM | |
| Human liver cancer cell line: | Dalian Meilun Biotech Co., Ltd. |
| HEP3B2.7-1 | |
| Human Burkitt's lymphoma cell line: | Dalian Meilun Biotech Co., Ltd. |
| Daudi | |
| Human acute lymphoblastic leukemia | ATCC |
| cell line: Kasumi-1 | |
| Human diffuse large B-cell lymphoma | ATCC |
| cell line: SU-DHL-6 | |
| Human diffuse large B-cell lymphoma | Kyinno Biotechnology Co., |
| cell line: TMD8 | Ltd (test) |
| Human prostate cancer cell line: 22RV1 | Shanghai Medicilon Inc. (test) |
| Human prostate cancer cell line: C4-2 | Shanghai Medicilon Inc. (test) |
| CEREBLON BINDING KITS kit | Cisbio Company |
| F-12K Medium | ATCC |
| EMEM Medium | Dalian Meilun Biotech Co., Ltd. |
| Meilun Cell Counting Kit-8 | Dalian Meilun Biotech Co., Ltd. |
| Fetal bovine serum (FBS) | Sunrise Science Products |
| | Company |
| Penicillin-Streptomycin | Beijing TransGen Biotech |
| | Co., Ltd. |

### Methods

### Determination of CRBN-Binding Affinity of Compounds

The CEREBLON BINDING kits (specification: 10,000 tests; product number: Catalog #64BDCRBNPEH; CISBIO company) was used to determine the CRBN binding ability of the compounds to be tested through a HTRF method (homogeneous time-resolved fluorescence). The specific methods are as follows:
1. According to the instructions of the CEREBLON BINDING kits, the compounds to be tested of the present invention and lenalidomide were serially diluted using diluent #9 (1×) solution to obtain a final concentration of 2 M for both the tested compounds and lenalidomide solution.
2. 2.5 μL of the above 2 M of the tested compounds and lenalidomide solution, as well as the same volume of diluent #9 (1×) solution were taken and added to each well of a 96-well plate, respectively. Then, 2.5 μL of (human Cereblon WT GST-tagged protein) solution was added to each well. Finally, 5 μL of the uniformly mixed thalidomide-Red reagent and GST Eu antibody working solution were added to each of the above wells. The final concentration of the tested compounds and lenalidomide in each well is 0.5 μM.
3. The blank control wells were sequentially added with 2.5 μL of diluent #9 (1×) solution, 2.5 μL of ligand binding buffer, and 5 μL of uniformly mixed thalidomide-Red reagent and GST Eu antibody working solution.
4. After sealing and incubating the solutions in the above wells at room temperature for 3 hours, the absorbance values at emission wavelengths of 620 nm and 665 nm were detected by the HTRF method using a Spark microplate reader (V3.1 SP1).
   The corresponding CRBN binding inhibition rate was calculated using the following method:

$${R}_{compound} = OD 665 {nm}_{compound} / OD 620 {nm}_{compound} - OD 665 {nm}_{control} / OD 620 {nm}_{control}$$

$${R}_{Std 0} = OD 665 {nm}_{Std 0} / OD 620 {nm}_{Std 0} - OD 665 {nm}_{control} / OD 620 {nm}_{control}$$

$$inhibition rate ( \% ) = ( 1 - {R}_{compound} / {R}_{Std 0} ) * 100$$

Note: OD 665 nm _{compound} is the absorbance value of each well of the compounds to be tested of the present disclosure at an emission wavelength of 665 nm.
OD 620 nm _{compound} is the absorbance value of each well of the compounds to be tested of the present disclosure at an emission wavelength of 620 nm.
OD 665 nm control is the absorbance value of the blank control well at an emission wavelength of 665 nm.
OD 620 nm _{control} is the absorbance value of the blank control well at an emission wavelength of 620 nm.
OD 665 nm _{std0} is the absorbance value of the solvent control well at an emission wavelength of 665 nm.
OD 620 nm _{std0} is the absorbance value of the solvent control well at an emission wavelength of 620 nm.

The test results show that the CRBN binding activity IC₅₀ value of lenalidomide (Lena-NH₂) is 2.38 M. The binding ability of the compounds of the present invention to CRBN is superior to or equivalent to that of lenalidomide. The results of the binding affinity test are shown in Table 5 below.

TABLE 5
HTRF inhibitory activity (IC₅₀, μM)
of the compounds of the present invention

| Compound No. | HTRF IC₅₀ (μM) | Compound No. | HTRF IC₅₀ (μM) |
|---|---|---|---|
| Lena-NH₂ | 2.38 | GT-03100 | 9.80 |
| GT-03106 | 4.04 | GT-03075 | 5.09 |
| GT-03109 | 3.55 | GT-03210 | 2.69 |
| GT-03077 | 5.20 | GT-03211 | 9.80 |
| GT-03108 | 8.75 | GT-03076 | 0.65 |

Determination of half inhibitory concentration (IC₅₀) of the compounds against tumor cells:

### Cell Cultures

The tumor cells used herein were cultured in a cell culture medium listed in table 6 at 37° C. in an incubator with 5% CO₂. Prior to experimentation, all cells used were correctly identified by STR profiling, and tested negative for mycoplasma through routine screenings.

TABLE 6
Tumor cells and cell culture medium used in the present disclosure

| Cell line | Cell culture medium used |
|---|---|
| CCRF-CEM | RPMI 1640 culture medium supplemented |
| Kasumi-1 | with 10% FBS, as well as penicillin at |
| WSU-DLCL2 | a final concentration of 100 U/mL and |
| MM.1R | streptomycin at a final concentration |
| MGC80-3 | of 100 μg/mL |
| Daudi | |
| SU-DHL-6 | |
| TMD8 | |
| SW620 | L-15 culture medium supplemented with |
| | 10% FBS, as well as penicillin at a final |
| | concentration of 100 U/mL and streptomycin |
| | at a final concentration of 100 μg/mL |
| DU-145 | EMEM culture medium supplemented with |
| | 10% FBS, as well as penicillin at a final |
| | concentration of 100 U/mL and streptomycin |
| | at a final concentration of 100 μg/mL |
| LNcap | F-12K culture medium supplemented with |
| PC-3 | 10% FBS, as well as penicillin at a final |
| A549 | concentration of 100 U/mL and streptomycin |
| | at a final concentration of 100 μg/mL |
| HEP3B2.7-1 | MEM culture medium supplemented with 10% |
| | FBS, as well as penicillin at a final |
| | concentration of 100 U/mL and streptomycin |
| | at a final concentration of 100 μg/mL |

### Assay Method:

IC₅₀ values of the compounds of the present disclosure (including the compounds in Table 4 and compounds of examples 1-72) were measured using the Meilun Cell Counting Kit-S kit from Dalian Meilun Biotech Co., Ltd. Assay details are as follows: tumor cells were seeded in a 96-well plate at a density listed in Table 7. After 24 h, 100 μL of the inoculated cells were treated with 0.5 μL of the compounds of the present disclosure (including the compounds in Table 4 and compounds of examples 1-72) at 10 different successively decreasing concentrations (starting at the highest concentration of 10 μM; 5-fold serial dilutions). After the cells were treated with the compounds of the present disclosure for a period of time (the treatment times listed in Table 7), the cell viability detection reagent was added to the culture medium for cell viability assessment according to the instructions provided with the CCK-8 kit. DMSO served as the negative control, and corresponding commercial inhibitors (including Enzalutamide (CAS No.: 915087-33-1); Ribociclib (CAS No.: 1211443-58-1); Dasatinib (CAS No.: 302962-49-8)) were used as the positive control, both of which treated the cells using the same protocol as that of the compounds of the present disclosure. The growth inhibition of the compounds of the present disclosure on cells was plotted by Prism Graphpad software, and the IC₅₀ values of the compounds of the present disclosure were calculated therefrom. Results were shown in Tables 8-10 below.

TABLE 7
Seeding protocol and treatment time for tumor cells

| | | The times for |
|---|---|---|
| | | treating cells |
| | | with the |
| | | compounds of |
| | | the present |
| Cells | Cells seeding protocol | disclosure (hours) |
| Kasumi-1 | cells were seeded in 100 μL | 72 h |
| MM.1R | RPMI1640 medium containing serum | |
| SU-DHL-6 | at a density of 10,000 cells/well | |
| TMD8 | | |
| CCRF-CEM | cells were seeded in 100 μL | 96 h |
| WSU-DLCL2 | RPMI1640 medium containing serum | |
| MGC80-3 | at a density of 4000 cells/well | |
| Daudi | | |
| DU-145 | cells were seeded in 100 μL | 96 h |
| | EMEM medium containing serum at | |
| | a density of 4000 cells/well | |
| SW620 | cells were seeded in 100 μL | 96 h |
| | L-15 medium containing serum at | |
| | a density of 4000 cells/well | |
| LNcap | cells were seeded in 100 μL | 96 h |
| PC-3 | F-12K medium containing serum | |
| A549 | at a density of 4000 cells/well | |
| HEP3B2.7-1 | cells were seeded in 100 μL | 96 h |
| | MEM medium containing serum at | |
| | a density of 4000 cells/well | |

TABLE 8
IC₅₀ values (in nM) of the compounds of examples of the present disclosure, designed
based on the AR inhibitors, for inhibiting the proliferation of tumor cells

| Comp. No./ | | | | | | | | | | |
|---|---|---|---|---|---|---|---|---|---|---|
| names | LNcap | PC-3 | DU-145 | MM.1R | WSU-DLCL2 | MGC80-3 | SW620 | A549 | CCRF-CEM | Hep3B2.1-7 |
| Enzalutamide | — | >10000 | >10000 | >10000 | >10000 | — | >10000 | — | — | — |
| GT-03339 | B | B | B | — | B | A | — | — | — | — |
| GT-03490 | B | — | D | B | B | B | — | — | — | — |
| GT-03488 | A+ | — | A | A+ | — | A+ | — | A | — | A+ |
| GT-03489 | B | — | D | C | B | B | — | — | — | B |
| GT-03486 | B | — | — | B | B | A | — | B | B | — |
| GT-03487 | B | — | D | B | B | B | — | — | — | — |
| GT-02765 | A+ | A+ | A | B | A+ | A+ | A+ | A+ | | A+ |
| GT-03773 | — | — | — | — | — | — | B | — | — | — |
| GT-03016 | C | C | C | D | D | B | A | — | — | — |
| GT-03449 | A | — | C | B | — | A | — | — | B | A |
| GT-03450 | B | — | D | B | C | B | — | — | — | — |
| GT-03451 | B | — | D | B | B | C | — | — | — | — |
| GT-03427 | C | — | D | — | B | B | — | — | — | — |
| GT-03471 | C | — | — | — | D | B | — | — | — | B |
| GT-03425 | A | — | C | B | A | A+ | — | — | — | A+ |
| GT-03426 | A | — | — | B | — | A | — | — | — | A+ |
| GT-03481 | B | — | — | — | B | B | — | B | — | A+ |
| GT-03403 | C | B | B | — | E | C | — | — | — | — |
| GT-03404 | C | — | — | — | — | B | — | — | — | B |
| GT-03402 | A | A | A | — | B | — | — | B | — | — |
| GT-03482 | A | — | B | B | B | A | — | — | — | B |
| GT-03494 | A | — | D | — | A | A | — | B | — | A |
| GT-03400 | B | B | B | — | C | B | — | — | — | — |
| GT-03401 | C | B | C | — | D | B | — | — | — | — |
| GT-03399 | B | B | B | — | C | — | — | — | — | B |
| GT-03398 | A | A+ | A | — | — | A+ | — | A | — | — |

TABLE 9
IC₅₀ values (in nM) of the compounds of examples of the
present disclosure, designed based on the CDK4/6 inhibitors,
for inhibiting the proliferation of tumor cells

| Comp. No./names | SW620 |
|---|---|
| Ribociclib | >10000 |
| GT-03651 | B |

TABLE 10
IC₅₀ values (in nM) of the compounds of examples of the
present disclosure, designed based on the BCR-ABL inhibitors,
for inhibiting the proliferation of tumor cells

| Comp. No./names | Daudi | Kasumi-1 | SU-DHL-6 | TMD8 |
|---|---|---|---|---|
| Dasatinib | B | A+ | A+ | A+ |
| GT-03630 | — | A+ | B | — |
| GT-03628 | — | B | — | — |
| GT-03631 | B | B | A+ | — |
| GT-03538 | B | B | A+ | A+ |
| GT-03629 | — | B | B | — |
| GT-03627 | — | B | B | — |
| GT-03632 | A | A+ | A+ | — |
| GT-03537 | B | A | A+ | A+ |

Note:
In Tables 8-10, the symbols A+, A, B, C, D, and E are defined as follows: A+ ≤ 50 nM; 50 nM < A ≤ 100 nM; 100 nM < B ≤ 500 nM; 500 nM < C ≤ 1000 nM; 1000 nM < D ≤ 5000 nM; 5000 nM < E ≤ 10000 nM;
symbol “—” represents undetected.

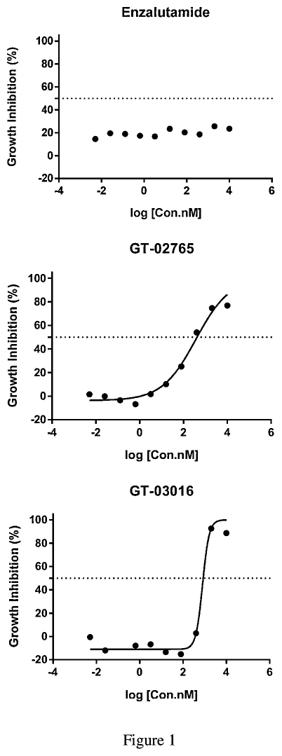

The results indicate that the compounds of the present invention (including the compounds in Table 4 and Examples 1-72) can inhibit the proliferation of tumor cells (as shown in Tables 8-10 and FIG. 1), with inhibitory effects superior to or comparable with the corresponding positive control drugs (including enzalutamide, ribociclib, and dasatinib), as well as degradation effect better than that of the corresponding positive control drugs.

### Determination of Half-Maximal Target Protein Degradation Concentration (DC₅₀) of the Compounds of the Present Disclosure to Target Proteins

### Western-Blot Assay

(1) Cell seeding: tumor cells (human prostate cancer cells 22RV1 or human prostate cancer cells C₄₋₂) were added to a 6-well plate at a cell density of 2.5×10⁵/mL. The total volume of cell suspension in each well is 2 mL. The compounds of the present disclosure (including the compounds in table 4 and the compounds of example 1-72) to be tested were set at 5 concentration gradients of 1 nM, 2 nM, 10 nM, 20 nM and 50 nM, respectively. Meanwhile, DMSO solvent (containing 0 nM of the compounds to be tested) was set as a negative control.

(2) Protein collecting: after treating the cells with the compounds for 24 hours, the cell culture medium was removed. The cells were washed with 1 ml of PBS, followed by removing the PBS and sequentially addition of 100 μL of RAPI lysis buffer (containing protease inhibitors). The mixture was placed on ice. The lysed cells were scraped off using a cell scraper, and transferred into a 1.5 m1 EP tube. Following this, the tube was placed on ice for 30-60 min, and centrifuged at 12,000 rpm for 5 min. The supernatant was aspirated as the extracted total cell protein. Protein concentrations were measured using the Bradford method, and bromophenol blue was incorporated as a loading dye.

(3) Electrophoresis: 15 μg of the proteins collected after cell lysis were loaded for electrophoresis. Electrophoresis set at a constant voltage of 80V for 15 min, and once the dye entered the separating gel, the voltage was adjusted to 120V for 40 min.

(4) Membrane transfer: filter paper and nitrocellulose membrane (NC membrane) were prepared in an appropriate size. Then the filter paper and NC membrane were soaked in the transfer electrophoresis buffer. They were then placed in the electrophoresis tank in the following order: “filter paper-gel-NC membrane-filter paper,” and the membrane was transferred with a constant current of 400 mA for 1 h. Subsequent antibody incubation and development are performed according to the antibody instructions provided by Cell Signaling Technology.

Determining Half-Maximal Degradation Concentration (DC₅₀) value (the drug concentration required for degrading proteins by 50%, abbreviated as DC₅₀) reads method: comparing the grayscale values of the Western blotting bands for the drug treatment with those of the Western blotting bands after treatment with blank DMSO. The DC₅₀ is identified within the range of drug concentrations where the grayscale value matches half of the grayscale value of the corresponding Western blotting band after treatment with blank DMSO.

Calculation of the DC₅₀ values can be performed using ImageJ to measure the grayscale values of Western blotting bands after drug treatment. A curve fitting the relationship between drug concentration and grayscale values is then generated to estimate the drug concentration corresponding to half of the grayscale value. A curve is then fitted to the relationship between drug concentration and grayscale values, allowing estimation of the drug concentration that corresponds to half of the grayscale value.

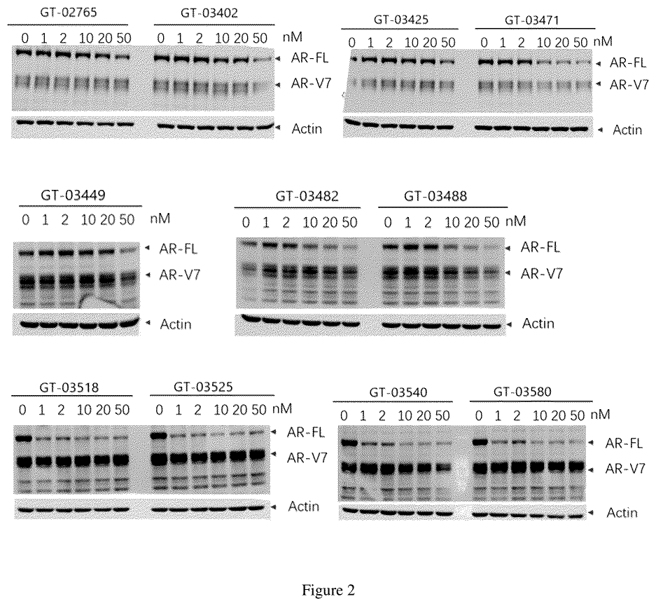

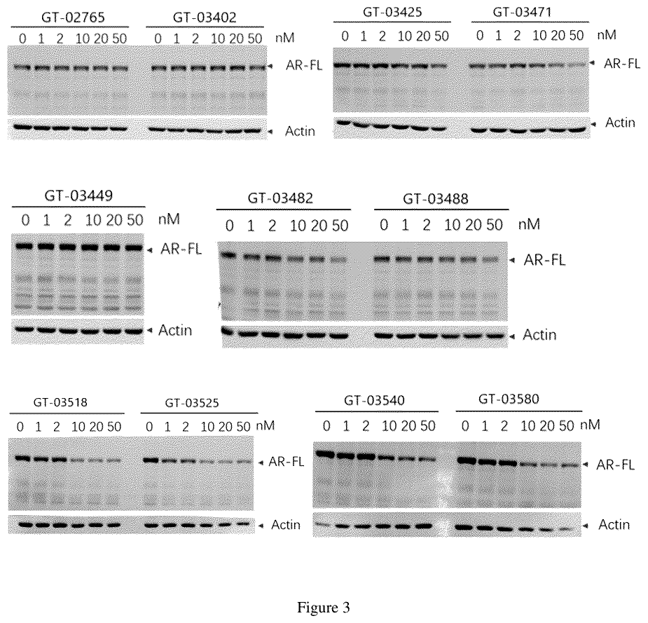

The present invention assessed the effects of the compounds of the present disclosure (including compounds in Table 4 and compounds of examples 1-72) on the protein expression levels of full-length androgen receptor (AR-FL) and androgen receptor splice variant 7 (AR-V7) in human prostate cancer cell lines 22RV1 and C4-2. Human prostate cancer cell line 22RV1 highly expresses AR-FL and AR-V7 splice variant, while human prostate cancer cell line C4-2 highly expresses AR-FL. The expression of AR-V7 splice variant, which lacks the LBD domain, represents one of the significant mechanisms that contribute to resistance against the AR antagonist enzalutamide. The Western-blot detection results were shown in FIGS. 2-3.

Experimental results demonstrate that, compared with the DMSO control group, the compounds of the present invention (including compounds in Table 4 and compounds of examples 1-72) exhibit a significant degradation effect on both AR-FL and the AR-V7 splice variant.

The basic principles, main features and advantages of the present disclosure are shown and described above. Those skilled in the art should understand that the present disclosure is not limited by the foregoing embodiments, and they can make various changes, substitutions and alterations herein without departing from the spirit and scope of the present disclosure. These changes, substitutions and alterations fall within the scope of the present disclosure. The claimed scope of the present disclosure is defined by the appended claims and their equivalents.

1. A compound of Formula (I) 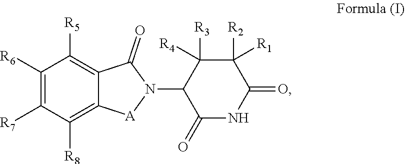
or salts, enantiomers, stereoisomers, solvates, isotopically enriched analogs, prodrugs, or polymorphs thereof,
wherein A represents CO, CH₂ or CD₂;
R₁, R₂, R₃ and R₄ are the same or different and each independently represent hydrogen, deuterium, halogen, optionally deuterated C₁₋₆ alkyl, optionally deuterated C₁₋₆ alkoxy, or halogenated C₁₋₆ alkyl;
R₅, R₆, R₇ and R₈ are the same or different and each independently represent R₉, hydrogen, deuterium, halogen, hydroxy, mercapto, nitro, amino, cyano, optionally deuterated C₁₋₆ alkyl, optionally deuterated C₁₋₆ alkoxy, halogenated C₁₋₆ alkyl, C₂₋₆ alkenyl, or C₂₋₆ alkynyl, wherein one to three of R₅, R₆, R₇ and R₈ represents R₉;
R₉ represents R—U— group, wherein
U represents a bond, or U represents N(R₁₀) or O, wherein R₁₀ represents H or optionally deuterated C₁₋₆ alkyl;
R represents optionally substituted nitrogen-containing heterocyclyl, said optionally substituted nitrogen-containing heterocyclyl is optionally substituted nitrogen-containing monocyclic heterocyclyl or optionally substituted nitrogen-containing bridged heterocyclyl;
wherein
when two to four of R₅, R₆, R₇, and R₈ independently represent R₉, the compound of Formula (I) is optionally partially or fully deuterated; or
when only one of R₅, R₆, R₇, and R₈ represents R₉, the compound of Formula (I) is optionally partially or fully deuterated;
wherein in case that only one of R₅, R₆, R₇, and R₈ represents R₉ and the compound of Formula (I) is not deuterated:
when R represents optionally substituted nitrogen-containing bridged heterocyclyl, the following compounds are excluded:
5-(2,5-diazabicyclo[2.2.1]heptan-2-yl)-2-(2,6-dioxopiperidin-3-yl)isoindoline-1,3-dione;
5-(2,5-diazabicyclo[2.2.1]heptan-2-yl)-2-(2,6-dioxopiperidin-3-yl)-6-fluoroisoindoline-1,3-dione;
5-(2,5-diazabicyclo[2.2.1]heptan-2-yl)-2-(2,6-dioxopiperidin-3-yl)-6-fluoroisoindoline-1,3-dione;
3-(5-(2,5-diazabicyclo[2.2.1]heptan-2-yl)-1-oxoisoindolin-2-yl)piperidine-2,6-dione;
3-(5-(8-azabicyclo[3.2.1]octan-3-yl)-1-oxoisoindolin-2-yl)piperidine-2,6-dione; and
3-(5-(9-ethyl-3-methyl-3,9-diazabicyclo[3.3.1]nonan-7-yl)-1-oxoisoindolin-2-yl)piperidine-2,6-dione; or
when A represents CO and one of R₆ and R₇ represents R₉, where R represents substituted nitrogen-containing monocyclic heterocyclyl, and the other one of R₆ and R₇, as well as R₅ and R₈ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, one to three of the other one of R₆ and R₇ as well as R₅ and R₈ represents halogen, and the nitrogen-containing monocyclic heterocyclyl is substituted with one or more substituents selected from the group consisting of hydroxy, amino, mercapto, nitro, halogen, cyano, C₁₋₆ alkyl, C₁₋₆ alkoxy, halogenated C₁₋₆ alkyl, NH₂—C₁₋₆ alkylene, C₁₋₆ alkyl-NH—, C₁₋₆ alkyl-NHC(O)—, C₁₋₆ alkyl-C(O)NH— or any combination thereof;
when A represents CO and one of R₆ and R₇ represents R₉, where R represents unsubstituted nitrogen-containing monocyclic heterocyclyl, the other one of R₆ and R₇ represents hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, and R₅ and R₈ each independently represent halogen;
when A represents CO and one of R₅ and R₈ represents R₉, where R represents substituted nitrogen-containing monocyclic heterocyclyl, and the other one of R₅ and R₈, as well as R₆ and R₇each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, one to three of the other one of R₅ and R₈ as well as R₆ and R₇ represents halogen, and the nitrogen-containing monocyclic heterocyclyl is substituted with one or more substituents selected from the group consisting of hydroxy, amino, mercapto, nitro, halogen, cyano, CHO, C₁₋₆ alkyl, halogenated C₁₋₆ alkyl, C₃₋₆ cycloalkyl, C₁₋₆ alkoxy, C₁₋₆ alkyl-NH—, NH₂—C₁₋₆ alkylene, C₁₋₆ alkyl-NHC(O)—, C₁₋₆ alkyl-C(O)NH— or any combination thereof;
when A represents CO and one of R₅ and R₈ represents R₉, where R represents unsubstituted nitrogen-containing monocyclic heterocyclyl, the other one of R₅ and R₈ and/or R₇ each independently represent halogen, and R₆ represents hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl;
when A represents CH₂ and one of R₆ and R₇ represents R₉, where R represents substituted nitrogen-containing monocyclic heterocyclyl, and the other one of R₆ and R₇, as well as R₅ and R₈ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, one to three of the other one of R₆ and R₇ as well as R₅ and R₈ represents halogen, and the nitrogen-containing monocyclic heterocyclyl is substituted with one or more substituents selected from the group consisting of hydroxy, amino, mercapto, nitro, halogen, CHO, cyano, C₁₋₆ alkyl, halogenated C₁₋₆ alkyl, C₃₋₆ cycloalkyl, C₁₋₆ alkoxy, C₁₋₆ alkyl-NH—, NH₂—C₁₋₆ alkylene, C₁₋₆ alkyl-NHC(O)—, C₁₋₆ alkyl-C(O)NH— or any combination thereof;
when A represents CH₂ and one of R₆ and R₇ represents R₉, where R represents unsubstituted nitrogen-containing monocyclic heterocyclyl, R₅ represents halogen, and the other one of R₆ and R₇ and/or R₈ each independently represent halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl;
when A represents CH₂ and one of R₅ and R₈ represents R₉, where R represents substituted nitrogen-containing monocyclic heterocyclyl, and the other one of R₅ and R₈, as well as R₆ and R₇ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, one to three of the other one of R₅ and R₈ as well as R₆ and R₇ represent halogen, and the nitrogen-containing monocyclic heterocyclyl is substituted with one or more substituents selected from the group consisting of hydroxy, amino, mercapto, nitro, halogen, CHO, cyano, C₁₋₆ alkyl, halogenated C₁₋₆ alkyl, C₃₋₆ cycloalkyl, C₁₋₆ alkoxy, C₁₋₆ alkyl-NH—, NH₂—C₁₋₆ alkylene, C₁₋₆ alkyl-NHC(O)—, C₁₋₆ alkyl-C(O)NH— or any combination thereof; or
when A represents CH₂ and one of R₅ and R₈ represents R₉, where R represents unsubstituted nitrogen-containing monocyclic heterocyclyl, and the other one of R₅ and R₈, as well as R₆ and R₇ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, the other one of R₅ and R₈, as well as R₆ and R₇ are not simultaneously hydrogen, and when R₆ is halogen, the other one of R₅ and R₈, as well as R₇ are not simultaneously hydrogen.

2. The compound of Formula (I) or salts, enantiomers, stereoisomers, solvates, isotopically enriched analogs, prodrugs, or polymorphs thereof as claimed in claim 1, wherein the compound of Formula (I) is also of Formula (I-1) or Formula (I-2): 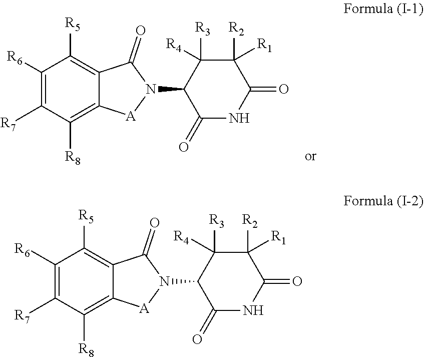
wherein A, R₁, R₂, R₃, R₄, R₅, R₆, R₇ and R₈ are as defined in claim 1.

3. The compound of Formula (I) or salts, enantiomers, stereoisomers, solvates, isotopically enriched analogs, prodrugs, or polymorphs thereof as claimed in claim 1, wherein (i) when two to four of R₅, R₆, R₇ and R₈ independently represent R₉, the compound of Formula (I) is optionally partially deuterated, for example, carrying 1 to 20 deuterium atom, or the compound of Formula (I) is optionally fully deuterated;
and/or
(ii) the nitrogen-containing monocyclic heterocyclyl or nitrogen-containing bridged heterocyclyl is optionally substituted with one or more substituents selected from the group consisting of deuterium, hydroxy, amino, mercapto, nitro, halogen, cyano, CHO, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof.

4. The compound of Formula (I) or salts, enantiomers, stereoisomers, solvates, isotopically enriched analogs, prodrugs, or polymorphs thereof as claimed in claim 1, wherein when only one of R₅, R₆, R₇ and R₈ represents R₉, the compound of Formula (I) is partially deuterated, for example, carrying 1 to 15 deuterium atom, or the compound of Formula (I) is fully deuterated.

5. (canceled)

6. The compound of Formula (I) or salts, enantiomers, stereoisomers, solvates, isotopically enriched analogs, prodrugs, or polymorphs thereof as claimed in claim 1, wherein
the nitrogen-containing monocyclic heterocyclyl is a 4- to 10-membered monocyclic heterocyclyl group containing one nitrogen atom and optionally a heteroatom selected from nitrogen, oxygen, and sulfur, e.g., piperidinyl, piperazinyl, morpholinyl, azetidinyl, pyrrolidinyl, imidazolidinyl, pyrazolidyl, oxazolidinyl, thiazolidinyl, thiomorpholinyl, azepanyl, diazacycloheptanyl, azacyclooctyl or diazacyclooctyl; or
the nitrogen-containing bridged heterocyclyl is a C₅₋₂₀ bridged heterocyclyl group containing one nitrogen atom and optionally a heteroatom selected from nitrogen, oxygen, and sulfur, e.g., azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl or diazabicyclo[2.2.2]octanyl, such as the following groups: 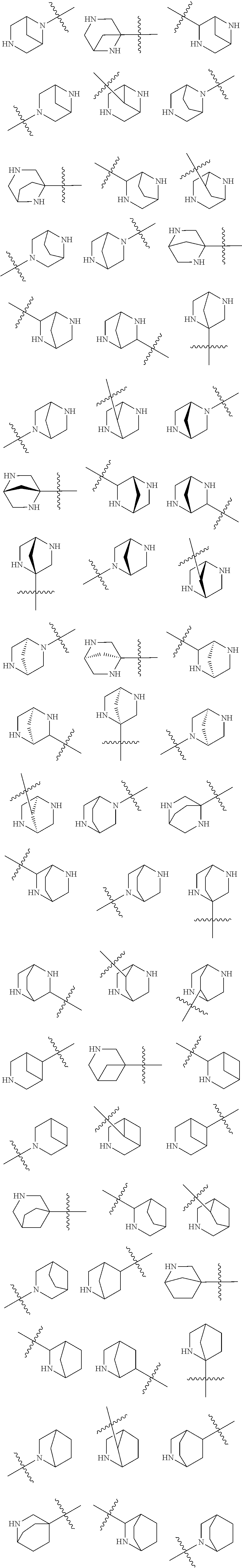 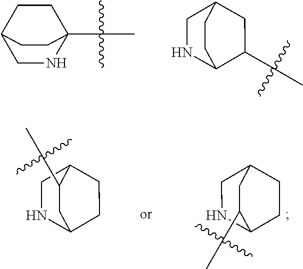 [SMILES: *C12CCC(CC1)CN2 ; *C1CC2CCC1NC2 ; *C1CC2CCC1CN2 ; "or" ; *C1CC2CCC1NC2 ; ";"]
wherein the nitrogen-containing monocyclic heterocyclyl and nitrogen-containing bridged heterocyclyl are optionally substituted with one or more substituents selected from the group consisting of deuterium, hydroxy, amino, mercapto, nitro, halogen, cyano, CHO, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof.

7. The compound of Formula (I) or salts, enantiomers, stereoisomers, solvates, isotopically enriched analogs, prodrugs, or polymorphs thereof as claimed in claim 1, wherein R₉ represents the following groups:

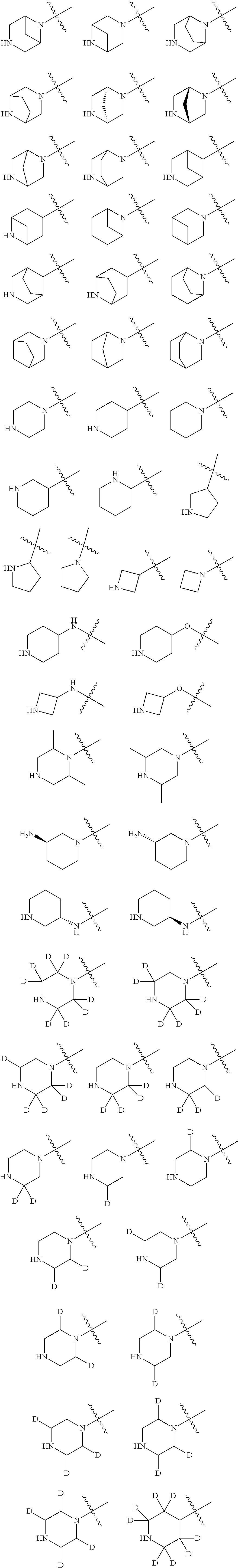

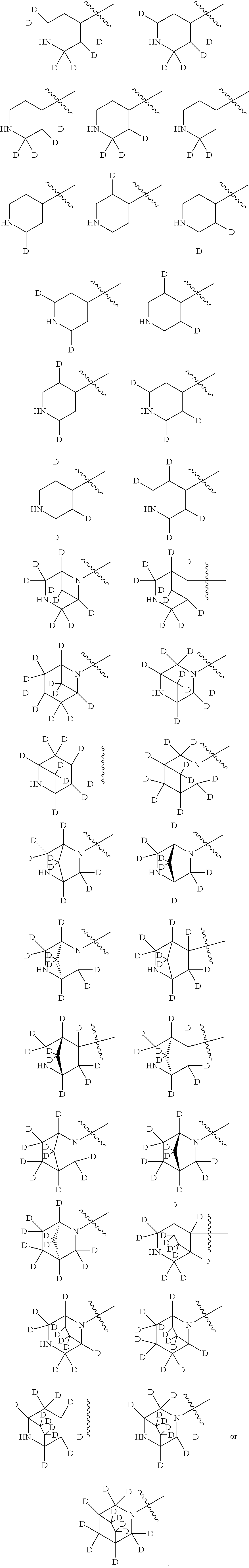

8. The compound of Formula (I) or salts, enantiomers, stereoisomers, solvates, isotopically enriched analogs, prodrugs, or polymorphs thereof as claimed in claim 1, which is selected from:
5-(3,6-diazabicyclo[3.1.1]heptan-6-yl)-2-(2,6-dioxopiperidin-3-yl)isoindoline-1,3-dione;
5-(3,6-diazabicyclo[3.1.1]heptan-3-yl)-2-(2,6-dioxopiperidin-3-yl)isoindoline-1,3-dione;
5-(3, 8-diazabicyclo[3.2.1]octan-8-yl)-2-(2,6-dioxopiperidin-3-yl)isoindoline-1,3-dione;
5-(3,8-diazabicyclo[3.2.1]octan-3-yl)-2-(2,6-dioxopiperidin-3-yl)isoindoline-1,3-dione;
5-((1S,4S)-2,5-diazabicyclo[2.2.1]heptan-2-yl)-2-(2,6-dioxopiperidin-3-yl)-6-fluoroisoindoline-1,3-dione;
5-(2,5-diazabicyclo[2.2.2]octan-2-yl)-2-(2,6-dioxopiperidin-3-yl)isoindoline-1,3-dione;
2-(2,6-dioxopiperidin-3-yl)-5-(piperazin-1-yl-2,2,3,3,5,5,6,6-d₈)isoindoline-1,3-dione;
5-(3-azabicyclo[3.1.1]heptan-6-yl)-2-(2,6-dioxopiperidin-3-yl)isoindoline-1,3-dione;
5-(6-azabicyclo[3.1.1]heptan-3-yl)-2-(2,6-dioxopiperidin-3-yl)isoindoline-1,3-dione;
5-(3-azabicyclo[3.2.1]octan-8-yl)-2-(2,6-dioxopiperidin-3-yl)isoindoline-1,3-dione;
5-(8-azabicyclo[3.2.1]octan-3-yl)-2-(2,6-dioxopiperidin-3-yl)isoindoline-1,3-dione;
5-(2-azabicyclo[2.2.1]heptan-5-yl)-2-(2,6-dioxopiperidin-3-yl)isoindoline-1,3-dione;
5-(2-azabicyclo[2.2.2]octan-5-yl)-2-(2,6-dioxopiperidin-3-yl)isoindoline-1,3-dione;
5-(3,6-diazabicyclo[3.1.1]heptan-6-yl)-2-(2,6-dioxopiperidin-3-yl)-6-fluoroisoindoline-1,3-dione;
5-(3,6-diazabicyclo[3.1.1]heptan-3-yl)-2-(2,6-dioxopiperidin-3-yl)-6-fluoroisoindoline-1,3-dione;
5-(3,8-diazabicyclo[3.2.1]octan-8-yl)-2-(2,6-dioxopiperidin-3-yl)-6-fluoroisoindoline-1,3-dione;
5-(3,8-diazabicyclo[3.2.1]octan-3-yl)-2-(2,6-dioxopiperidin-3-yl)-6-fluoroisoindoline-1,3-dione;
5-((1S,4S)-2,5-diazabicyclo[2.2.1]heptan-2-yl)-2-(2,6-dioxopiperidin-3-yl)-6-fluoroisoindoline-1,3-dione;
5-(2,5-diazabicyclo[2.2.2]octan-2-yl)-2-(2,6-dioxopiperidin-3-yl)-6-fluoroisoindoline-1,3-dione;
5-(2,6-dimethylpiperazin-1-yl)-2-(2,6-dioxopiperidin-3-yl)-6-fluoroisoindoline-1,3-dione;
5-(3,5-dimethylpiperazin-1-yl)-2-(2,6-dioxopiperidin-3-yl)-6-fluoroisoindoline-1,3-dione;
2-(2,6-dioxopiperidin-3-yl)-5-fluoro-6-(piperazin-1-yl-2,2,3,3,5,5,6,6-d₈)isoindoline-1,3-dione;
2-(2,6-dioxopiperidin-3-yl)-4-fluoro-5-(piperazin-1-yl-2,2,3,3,5,5,6,6-d₈)isoindoline-1,3-dione;
2-(2,6-dioxopiperidin-3-yl)-4-fluoro-6-(piperazin-1-yl-2,2,3,3,5,5,6,6-d₈)isoindoline-1,3-dione;
2-(2,6-dioxopiperidin-3-yl)-4,7-difluoro-5-(piperazin-1-yl-2,2,3,3,5,5,6,6-d₈)isoindoline-1,3-dione;
2-(2,6-dioxopiperidin-3-yl)-4,6-difluoro-5-(piperazin-1-yl-2,2,3,3,5,5,6,6-d₈)isoindoline-1,3-dione;
2-(2,6-dioxopiperidin-3-yl)-4,5-difluoro-6-(piperazin-1-yl-2,2,3,3,5,5,6,6-d₈)isoindoline-1,3-dione;
2-(2,6-dioxopiperidin-3-yl)-4,5,7-trifluoro-6-(piperazin-1-yl-2,2,3,3,5,5,6,6-d₈)isoindoline-1,3-dione;
5-(3,6-diazabicyclo[3.1.1]heptan-6-yl)-2-(2,6-dioxopiperidin-3-yl)-4-fluoroisoindoline-1,3-dione;
5-(3,6-diazabicyclo[3.1.1]heptan-3-yl)-2-(2,6-dioxopiperidin-3-yl)-4-fluoroisoindoline-1,3-dione;
5-(3,8-diazabicyclo[3.2.1]octan-8-yl)-2-(2,6-dioxopiperidin-3-yl)-4-fluoroisoindoline-1,3-dione;
5-(3,8-diazabicyclo[3.2.1]octan-3-yl)-2-(2,6-dioxopiperidin-3-yl)-4-fluoroisoindoline-1,3-dione;
5-(2,5-diazabicyclo[2.2.1]heptan-2-yl)-2-(2,6-dioxopiperidin-3-yl)-4-fluoroisoindoline-1,3-dione;
5-(2,5-diazabicyclo[2.2.2]octan-2-yl)-2-(2,6-dioxopiperidin-3-yl)-4-fluoroisoindoline-1,3-dione;
5-(2,6-dimethylpiperazin-1-yl)-2-(2,6-dioxopiperidin-3-yl)-4-fluoroisoindoline-1,3-dione;
5-(3,5-dimethylpiperazin-1-yl)-2-(2,6-dioxopiperidin-3-yl)-4-fluoroisoindoline-1,3-dione;
6-(3,6-diazabicyclo[3.1.1]heptan-6-yl)-2-(2,6-dioxopiperidin-3-yl)-4-fluoroisoindoline-1,3-dione;
6-(3,6-diazabicyclo[3.1.1]heptan-6-yl)-2-(2,6-dioxopiperidin-3-yl)-4,5-difluoroisoindoline-1,3-dione;
5-(3,6-diazabicyclo[3.1.1]heptan-6-yl)-2-(2,6-dioxopiperidin-3-yl)-4,7-difluoroisoindoline-1,3-dione;
5-(3,6-diazabicyclo[3.1.1]heptan-6-yl)-2-(2,6-dioxopiperidin-3-yl)-4,6-difluoroisoindoline-1,3-dione;
5-(3,6-diazabicyclo[3.1.1]heptan-6-yl)-2-(2,6-dioxopiperidin-3-yl)-4,6,7-trifluoroisoindoline-1,3-dione;
6-(3,6-diazabicyclo[3.1.1]heptan-3-yl)-2-(2,6-dioxopiperidin-3-yl)-4-fluoroisoindoline-1,3-dione;
6-(3,6-diazabicyclo[3.1.1]heptan-3-yl)-2-(2,6-dioxopiperidin-3-yl)-4,5-difluoroisoindoline-1,3-dione;
5-(3,6-diazabicyclo[3.1.1]heptan-3-yl)-2-(2,6-dioxopiperidin-3-yl)-4,7-difluoroisoindoline-1,3-dione;
5-(3,6-diazabicyclo[3.1.1]heptan-3-yl)-2-(2,6-dioxopiperidin-3-yl)-4,6-difluoroisoindoline-1,3-dione;
5-(3,6-diazabicyclo[3.1.1]heptan-3-yl)-2-(2,6-dioxopiperidin-3-yl)-4,6,7-trifluoroisoindoline-1,3-dione;
6-(3,8-diazabicyclo[3.2.1]octan-8-yl)-2-(2,6-dioxopiperidin-3-yl)-4-fluoroisoindoline-1,3-dione;
6-(3,8-diazabicyclo[3.2.1]octan-8-yl)-2-(2,6-dioxopiperidin-3-yl)-4,5-difluoroisoindoline-1,3-dione;
5-(3,8-diazabicyclo[3.2.1]octan-8-yl)-2-(2,6-dioxopiperidin-3-yl)-4,7-difluoroisoindoline-1,3-dione;
5-(3,8-diazabicyclo[3.2.1]octan-8-yl)-2-(2,6-dioxopiperidin-3-yl)-4,6-difluoroisoindoline-1,3-dione;
5-(3,8-diazabicyclo[3.2.1]octan-8-yl)-2-(2,6-dioxopiperidin-3-yl)-4,6,7-trifluoroisoindoline-1,3-dione;
6-(3,8-diazabicyclo[3.2.1]octan-3-yl)-2-(2,6-dioxopiperidin-3-yl)-4-fluoroisoindoline-1,3-dione;
6-(3,8-diazabicyclo[3.2.1]octan-3-yl)-2-(2,6-dioxopiperidin-3-yl)-4,5-difluoroisoindoline-1,3-dione;
5-(3,8-diazabicyclo[3.2.1]octan-3-yl)-2-(2,6-dioxopiperidin-3-yl)-4,7-difluoroisoindoline-1,3-dione;
5-(3,8-diazabicyclo[3.2.1]octan-3-yl)-2-(2,6-dioxopiperidin-3-yl)-4,6-difluoroisoindoline-1,3-dione;
5-(3,8-diazabicyclo[3.2.1]octan-3-yl)-2-(2,6-dioxopiperidin-3-yl)-4,6,7-trifluoroisoindoline-1,3-dione;
2-(2,6-dioxopiperidin-3-yl)-4,5,7-trifluoro-6-(piperazin-1-yl)isoindoline-1,3-dione;
6-(2,5-diazabicyclo[2.2.1]heptan-2-yl)-2-(2,6-dioxopiperidin-3-yl)-4-fluoroisoindoline-1,3-dione;
6-(2,5-diazabicyclo[2.2.1]heptan-2-yl)-2-(2,6-dioxopiperidin-3-yl)-4,5-difluoroisoindoline-1,3-dione;
5-(2,5-diazabicyclo[2.2.1]heptan-2-yl)-2-(2,6-dioxopiperidin-3-yl)-4,7-difluoroisoindoline-1,3-dione;
5-(2,5-diazabicyclo[2.2.1]heptan-2-yl)-2-(2,6-dioxopiperidin-3-yl)-4,6-difluoroisoindoline-1,3-dione;
5-(2,5-diazabicyclo[2.2.1]heptan-2-yl)-2-(2,6-dioxopiperidin-3-yl)-4,6,7-trifluoroisoindoline-1,3-dione;
6-(2,5-diazabicyclo[2.2.2]octan-2-yl)-2-(2,6-dioxopiperidin-3-yl)-4-fluoroisoindoline-1,3-dione;
5-(2,5-diazabicyclo[2.2.1]heptan-2-yl)-2-(2,6-dioxopiperidin-3-yl)-4,6-difluoroisoindoline-1,3-dione;
5-(2,5-diazabicyclo[2.2.2]octan-2-yl)-2-(2,6-dioxopiperidin-3-yl)-4,7-difluoroisoindoline-1,3-dione;
5-(2,5-diazabicyclo[2.2.2]octan-2-yl)-2-(2,6-dioxopiperidin-3-yl)-4,6-difluoroisoindoline-1,3-dione;
5-(2,5-diazabicyclo[2.2.2]octan-2-yl)-2-(2,6-dioxopiperidin-3-yl)-4,6,7-trifluoroisoindoline-1,3-dione;
6-(2,6-dimethylpiperazin-1-yl)-2-(2,6-dioxopiperidin-3-yl)-4-fluoroisoindoline-1,3-dione;
6-(2,6-dimethylpiperazin-1-yl)-2-(2,6-dioxopiperidin-3-yl)-4,5-difluoroisoindoline-1,3-dione;
5-(2,6-dimethylpiperazin-1-yl)-2-(2,6-dioxopiperidin-3-yl)-4,7-difluoroisoindoline-1,3-dione;
5-(2,6-dimethylpiperazin-1-yl)-2-(2,6-dioxopiperidin-3-yl)-4,6-difluoroisoindoline-1,3-dione;
5-(2,6-dimethylpiperazin-1-yl)-2-(2,6-dioxopiperidin-3-yl)-4,6,7-trifluoroisoindoline-1,3-dione;
6-(3,6-diazabicyclo[3.1.1]heptan-6-yl)-2-(2,6-dioxopiperidin-3-yl)-4-fluoroisoindoline-1,3-dione;
6-(3,6-diazabicyclo[3.1.1]heptan-3-yl)-2-(2,6-dioxopiperidin-3-yl)-4-fluoroisoindoline-1,3-dione;
6-(3,8-diazabicyclo[3.2.1]octan-8-yl)-2-(2,6-dioxopiperidin-3-yl)-4-fluoroisoindoline-1,3-dione;
6-(3,8-diazabicyclo[3.2.1]octan-3-yl)-2-(2,6-dioxopiperidin-3-yl)-4-fluoroisoindoline-1,3-dione;
6-(2,5-diazabicyclo[2.2.1]heptan-2-yl)-2-(2,6-dioxopiperidin-3-yl)-4-fluoroisoindoline-1,3-dione;
6-(2,5-diazabicyclo[2.2.2]octan-2-yl)-2-(2,6-dioxopiperidin-3-yl)-4-fluoroisoindoline-1,3-dione;
6-(2,6-dimethylpiperazin-1-yl)-2-(2,6-dioxopiperidin-3-yl)-4-fluoroisoindoline-1,3-dione;
6-(3,5-dimethylpiperazin-1-yl)-2-(2,6-dioxopiperidin-3-yl)-4-fluoroisoindoline-1,3-dione;
6-(3,5-dimethylpiperazin-1-yl)-2-(2,6-dioxopiperidin-3-yl)-4,5-difluoroisoindoline-1,3-dione;
5-(3,5-dimethylpiperazin-1-yl)-2-(2,6-dioxopiperidin-3-yl)-4,7-difluoroisoindoline-1,3-dione;
5-(3,5-dimethylpiperazin-1-yl)-2-(2,6-dioxopiperidin-3-yl)-4,6-difluoroisoindoline-1,3-dione;
5-(3,5-dimethylpiperazin-1-yl)-2-(2,6-dioxopiperidin-3-yl)-4,6,7-trifluoroisoindoline-1,3-dione;
3-(5-(3,6-diazabicyclo[3.1.1]heptan-6-yl)-1-oxoisoindolin-2-yl)piperidine-2,6-dione;
3-(5-(3,6-diazabicyclo[3.1.1]heptan-3-yl)-1-oxoisoindolin-2-yl)piperidine-2,6-dione;
3-(5-(3,8-diazabicyclo[3.2.1]octan-8-yl)-1-oxoisoindolin-2-yl)piperidine-2,6-dione;
3-(5-(3,8-diazabicyclo[3.2.1]octan-3-yl)-1-oxoisoindolin-2-yl)piperidine-2,6-dione;
3-(5-((1R,4R)-2,5-diazabicyclo[2.2.1]heptan-2-yl)-6-fluoro-1-oxoisoindolin-2-yl)piperidine-2,6-dione;
3-(5-((1S,4S)-2,5-diazabicyclo[2.2.1]heptan-2-yl)-6-fluoro-1-oxoisoindolin-2-yl)piperidine-2,6-dione;
3-(5-(2,5-diazabicyclo[2.2.2]octan-2-yl)-1-oxoisoindolin-2-yl)piperidine-2,6-dione;
3-(1-oxo-5-(piperazin-1-yl-2,2,3,3,5,5,6,6-d₈)isoindolin-2-yl)piperidine-2,6-dione;
3-(5-(3-azabicyclo[3.1.1]heptan-6-yl)-1-oxoisoindolin-2-yl)piperidine-2,6-dione;
3-(5-(6-azabicyclo[3.1.1]heptan-3-yl)-1-oxoisoindolin-2-yl)piperidine-2,6-dione;
3-(5-(3-azabicyclo[3.2.1]octan-8-yl)-1-oxoisoindolin-2-yl)piperidine-2,6-dione;
3-(5-(3,6-diazabicyclo[3.1.1]heptan-6-yl)-6-fluoro-1-oxoisoindolin-2-yl)piperidine-2,6-dione;
3-(5-(3,6-diazabicyclo[3.1.1]heptan-3-yl)-6-fluoro-1-oxoisoindolin-2-yl)piperidine-2,6-dione;
3-(5-(3,8-diazabicyclo[3.2.1]octan-8-yl)-6-fluoro-1-oxoisoindolin-2-yl)piperidine-2,6-dione;
3-(5-(3,8-diazabicyclo[3.2.1]octan-3-yl)-6-fluoro-1-oxoisoindolin-2-yl)piperidine-2,6-dione;
3-(5-(2,5-diazabicyclo[2.2.2]octan-2-yl)-6-fluoro-1-oxoisoindolin-2-yl)piperidine-2,6-dione;
3-(5-(2,6-dimethylpiperazin-1-yl)-6-fluoro-1-oxoisoindolin-2-yl)piperidine-2,6-dione;
3-(5-(3,5-dimethylpiperazin-1-yl)-6-fluoro-1-oxoisoindolin-2-yl)piperidine-2,6-dione;
3-(6-fluoro-1-oxo-5-(piperazin-1-yl-2,2,3,3,5,5,6,6-d₈)isoindolin-2-yl)piperidine-2,6-dione;
3-(5-(3,6-diazabicyclo[3.1.1]heptan-6-yl)-4-fluoro-1-oxoisoindolin-2-yl)piperidine-2,6-dione;
3-(5-(3,6-diazabicyclo[3.1.1]heptan-3-yl)-4-fluoro-1-oxoisoindolin-2-yl)piperidine-2,6-dione;
3-(5-(3,8-diazabicyclo[3.2.1]octan-8-yl)-4-fluoro-1-oxoisoindolin-2-yl)piperidine-2,6-dione;
3-(5-(3,8-diazabicyclo[3.2.1]octan-3-yl)-4-fluoro-1-oxoisoindolin-2-yl)piperidine-2,6-dione;
3-(5-(2,5-diazabicyclo[2.2.1]heptan-2-yl)-4-fluoro-1-oxoisoindolin-2-yl)piperidine-2,6-dione;
3-(5-(2,5-diazabicyclo[2.2.2]octan-2-yl)-4-fluoro-1-oxoisoindolin-2-yl)piperidine-2,6-dione;
3-(4-fluoro-1-oxo-5-(piperazin-1-yl-2,2,3,3,5,5,6,6-d₈)isoindolin-2-yl)piperidine-2,6-dione;
3-(5-(2,6-dimethylpiperazin-1-yl)-4-fluoro-1-oxoisoindolin-2-yl)piperidine-2,6-dione;
3-(5-(3,5-dimethylpiperazin-1-yl)-4-fluoro-1-oxoisoindolin-2-yl)piperidine-2,6-dione;
3-(5-(3,6-diazabicyclo[3.1.1]heptan-6-yl)-7-fluoro-1-oxoisoindolin-2-yl)piperidine-2,6-dione;
3-(5-(3,6-diazabicyclo[3.1.1]heptan-3-yl)-7-fluoro-1-oxoisoindolin-2-yl)piperidine-2,6-dione;
3-(5-(3,8-diazabicyclo[3.2.1]octan-8-yl)-7-fluoro-1-oxoisoindolin-2-yl)piperidine-2,6-dione;
3-(5-(3,8-diazabicyclo[3.2.1]octan-3-yl)-7-fluoro-1-oxoisoindolin-2-yl)piperidine-2,6-dione;
3-(5-(2,5-diazabicyclo[2.2.1]heptan-2-yl)-7-fluoro-1-oxoisoindolin-2-yl)piperidine-2,6-dione;
3-(5-(2,5-diazabicyclo[2.2.2]octan-2-yl)-7-fluoro-1-oxoisoindolin-2-yl)piperidine-2,6-dione;
3-(7-fluoro-1-oxo-5-(piperazin-1-yl-2,2,3,3,5,5,6,6-d₈)isoindolin-2-yl)piperidine-2,6-dione;
3-(5-(2,6-dimethylpiperazin-1-yl)-7-fluoro-1-oxoisoindolin-2-yl)piperidine-2,6-dione;
3-(5-(3,5-dimethylpiperazin-1-yl)-7-fluoro-1-oxoisoindolin-2-yl)piperidine-2,6-dione;
3-(6,7-difluoro-1-oxo-5-(piperazin-1-yl)isoindolin-2-yl)piperidine-2,6-dione;
3-(4,7-difluoro-1-oxo-5-(piperazin-1-yl)isoindolin-2-yl)piperidine-2,6-dione;
3-(4,6,7-trifluoro-1-oxo-5-(piperazin-1-yl)isoindolin-2-yl)piperidine-2,6-dione;
3-(5-(3,6-diazabicyclo[3.1.1]heptan-6-yl)-7-fluoro-1-oxoisoindolin-2-yl)piperidine-2,6-dione;
3-(5-(3,6-diazabicyclo[3.1.1]heptan-6-yl)-6,7-difluoro-1-oxoisoindolin-2-yl)piperidine-2,6-dione;
3-(5-(3,6-diazabicyclo[3.1.1]heptan-6-yl)-4,7-difluoro-1-oxoisoindolin-2-yl)piperidine-2,6-dione;
3-(5-(3,6-diazabicyclo[3.1.1]heptan-6-yl)-4,6-difluoro-1-oxoisoindolin-2-yl)piperidine-2,6-dione;
3-(5-(3,6-diazabicyclo[3.1.1]heptan-6-yl)-4,6,7-trifluoro-1-oxoisoindolin-2-yl)piperidine-2,6-dione;
3-(5-(3,6-diazabicyclo[3.1.1]heptan-3-yl)-7-fluoro-1-oxoisoindolin-2-yl)piperidine-2,6-dione;
3-(5-(3,6-diazabicyclo[3.1.1]heptan-3-yl)-6,7-difluoro-1-oxoisoindolin-2-yl)piperidine-2,6-dione;
3-(5-(3,6-diazabicyclo[3.1.1]heptan-3-yl)-4,7-difluoro-1-oxoisoindolin-2-yl)piperidine-2,6-dione;
3-(5-(3,6-diazabicyclo[3.1.1]heptan-3-yl)-4,6-difluoro-1-oxoisoindolin-2-yl)piperidine-2,6-dione;
3-(5-(3,6-diazabicyclo[3.1.1]heptan-3-yl)-4,6,7-trifluoro-1-oxoisoindolin-2-yl)piperidine-2,6-dione;
3-(5-(3,8-diazabicyclo[3.2.1]octan-8-yl)-7-fluoro-1-oxoisoindolin-2-yl)piperidine-2,6-dione;
3-(5-(3,8-diazabicyclo[3.2.1]octan-8-yl)-6,7-difluoro-1-oxoisoindolin-2-yl)piperidine-2,6-dione;
3-(5-(3,8-diazabicyclo[3.2.1]octan-8-yl)-4,7-difluoro-1-oxoisoindolin-2-yl)piperidine-2,6-dione;
3-(5-(3,8-diazabicyclo[3.2.1]octan-8-yl)-4,6-difluoro-1-oxoisoindolin-2-yl)piperidine-2,6-dione;
3-(5-(3,8-diazabicyclo[3.2.1]octan-8-yl)-4,6,7-trifluoro-1-oxoisoindolin-2-yl)piperidine-2,6-dione;
3-(5-(3,8-diazabicyclo[3.2.1]octan-3-yl)-7-fluoro-1-oxoisoindolin-2-yl)piperidine-2,6-dione;
3-(5-(3,8-diazabicyclo[3.2.1]octan-3-yl)-6,7-difluoro-1-oxoisoindolin-2-yl)piperidine-2,6-dione;
3-(5-(3,8-diazabicyclo[3.2.1]octan-3-yl)-4,7-difluoro-1-oxoisoindolin-2-yl)piperidine-2,6-dione;
3-(5-(3,8-diazabicyclo[3.2.1]octan-3-yl)-4,6-difluoro-1-oxoisoindolin-2-yl)piperidine-2,6-dione;
3-(5-(3,8-diazabicyclo[3.2.1]octan-3-yl)-4,6,7-trifluoro-1-oxoisoindolin-2-yl)piperidine-2,6-dione;
3-(5-(2,5-diazabicyclo[2.2.1]heptan-2-yl)-7-fluoro-1-oxoisoindolin-2-yl)piperidine-2,6-dione;
3-(5-(2,5-diazabicyclo[2.2.1]heptan-2-yl)-6,7-difluoro-1-oxoisoindolin-2-yl)piperidine-2,6-dione;
3-(5-(2,5-diazabicyclo[2.2.1]heptan-2-yl)-4,7-difluoro-1-oxoisoindolin-2-yl)piperidine-2,6-dione;
3-(5-(2,5-diazabicyclo[2.2.1]heptan-2-yl)-4,6-difluoro-1-oxoisoindolin-2-yl)piperidine-2,6-dione;
3-(5-(2,5-diazabicyclo[2.2.1]heptan-2-yl)-4,6,7-trifluoro-1-oxoisoindolin-2-yl)piperidine-2,6-dione;
3-(5-(2,5-diazabicyclo[2.2.2]octan-2-yl)-7-fluoro-1-oxoisoindolin-2-yl)piperidine-2,6-dione;
3-(5-(2,5-diazabicyclo[2.2.2]octan-2-yl)-6,7-difluoro-1-oxoisoindolin-2-yl)piperidine-2,6-dione;
3-(5-(2,5-diazabicyclo[2.2.2]octan-2-yl)-4,7-difluoro-1-oxoisoindolin-2-yl)piperidine-2,6-dione;
3-(5-(2,5-diazabicyclo[2.2.2]octan-2-yl)-4,6-difluoro-1-oxoisoindolin-2-yl)piperidine-2,6-dione;
3-(5-(2,5-diazabicyclo[2.2.2]octan-2-yl)-4,6,7-trifluoro-1-oxoisoindolin-2-yl)piperidine-2,6-dione;
3-(7-fluoro-1-oxo-5-(piperazin-1-yl-2,2,3,3,5,5,6,6-d₈)isoindolin-2-yl)piperidine-2,6-dione;
3-(6,7-difluoro-1-oxo-5-(piperazin-1-yl-2,2,3,3,5,5,6,6-d₈)isoindolin-2-yl)piperidine-2,6-dione;
3-(4,7-difluoro-1-oxo-5-(piperazin-1-yl-2,2,3,3,5,5,6,6-d₈)isoindolin-2-yl)piperidine-2,6-dione;
3-(4,6-difluoro-1-oxo-5-(piperazin-1-yl-2,2,3,3,5,5,6,6-d₈)isoindolin-2-yl)piperidine-2,6-dione;
3-(4,6,7-trifluoro-1-oxo-5-(piperazin-1-yl-2,2,3,3,5,5,6,6-d₈)isoindolin-2-yl)piperidine-2,6-dione;
3-(5-(2,6-dimethylpiperazin-1-yl)-6,7-difluoro-1-oxoisoindolin-2-yl)piperidine-2,6-dione;
3-(5-(2,6-dimethylpiperazin-1-yl)-4,6-difluoro-1-oxoisoindolin-2-yl)piperidine-2,6-dione;
3-(5-(2,6-dimethylpiperazin-1-yl)-4,7-difluoro-1-oxoisoindolin-2-yl)piperidine-2,6-dione;
3-(5-(2,6-dimethylpiperazin-1-yl)-4,6,7-trifluoro-1-oxoisoindolin-2-yl)piperidine-2,6-dione;
3-(5-(3,5-dimethylpiperazin-1-yl)-6,7-difluoro-1-oxoisoindolin-2-yl)piperidine-2,6-dione;
3-(5-(3,5-dimethylpiperazin-1-yl)-4,7-difluoro-1-oxoisoindolin-2-yl)piperidine-2,6-dione;
3-(5-(3,5-dimethylpiperazin-1-yl)-4,6-difluoro-1-oxoisoindolin-2-yl)piperidine-2,6-dione;
3-(5-(3,5-dimethylpiperazin-1-yl)-4,6,7-trifluoro-1-oxoisoindolin-2-yl)piperidine-2,6-dione;
4-(3,6-diazabicyclo[3.1.1]heptan-6-yl)-2-(2,6-dioxopiperidin-3-yl)isoindoline-1,3-dione;
4-(3,6-diazabicyclo[3.1.1]heptan-3-yl)-2-(2,6-dioxopiperidin-3-yl)isoindoline-1,3-dione;
4-((1R,5S)-3,8-diazabicyclo[3.2.1]octan-8-yl)-2-(2,6-dioxopiperidin-3-yl)isoindoline-1,3-dione;
4-(2,5-diazabicyclo[2.2.1]heptan-2-yl)-2-(2,6-dioxopiperidin-3-yl)isoindoline-1,3-dione;
4-(2,5-diazabicyclo[2.2.2]octan-2-yl)-2-(2,6-dioxopiperidin-3-yl)isoindoline-1,3-dione;
4-((1R,5S)-3,8-diazabicyclo[3.2.1]octan-3-yl)-2-(2,6-dioxopiperidin-3-yl)isoindoline-1,3-dione;
2-(2,6-dioxopiperidin-3-yl)-4-(piperazin-1-yl-2,2,3,3,5,5,6,6-d₈)isoindoline-1,3-dione;
2-(2,6-dioxopiperidin-3-yl)-5-fluoro-4-(piperazin-1-yl)isoindoline-1,3-dione;
2-(2,6-dioxopiperidin-3-yl)-4-fluoro-7-(piperazin-1-yl)isoindoline-1,3-dione;
2-(2,6-dioxopiperidin-3-yl)-4,5-difluoro-7-(piperazin-1-yl)isoindoline-1,3-dione;
2-(2,6-dioxopiperidin-3-yl)-5,6-difluoro-4-(piperazin-1-yl)isoindoline-1,3-dione;
2-(2,6-dioxopiperidin-3-yl)-5,7-difluoro-4-(piperazin-1-yl)isoindoline-1,3-dione;
2-(2,6-dioxopiperidin-3-yl)-4,5,6-trifluoro-7-(piperazin-1-yl)isoindoline-1,3-dione;
4-(3,6-diazabicyclo[3.1.1]heptan-6-yl)-2-(2,6-dioxopiperidin-3-yl)-5-fluoroisoindoline-1,3-dione;
4-(3,6-diazabicyclo[3.1.1]heptan-6-yl)-2-(2,6-dioxopiperidin-3-yl)-6-fluoroisoindoline-1,3-dione;
4-(3,6-diazabicyclo[3.1.1]heptan-6-yl)-2-(2,6-dioxopiperidin-3-yl)-7-fluoroisoindoline-1,3-dione;
7-(3,6-diazabicyclo[3.1.1]heptan-6-yl)-2-(2,6-dioxopiperidin-3-yl)-4,5-difluoroisoindoline-1,3-dione;
4-(3,6-diazabicyclo[3.1.1]heptan-6-yl)-2-(2,6-dioxopiperidin-3-yl)-5,6-difluoroisoindoline-1,3-dione;
4-(3,6-diazabicyclo[3.1.1]heptan-6-yl)-2-(2,6-dioxopiperidin-3-yl)-5,7-difluoroisoindoline-1,3-dione;
4-(3,6-diazabicyclo[3.1.1]heptan-6-yl)-2-(2,6-dioxopiperidin-3-yl)-5,6,7-trifluoroisoindoline-1,3-dione;
4-(3,6-diazabicyclo[3.1.1]heptan-3-yl)-2-(2,6-dioxopiperidin-3-yl)-5-fluoroisoindoline-1,3-dione;
4-(3,6-diazabicyclo[3.1.1]heptan-3-yl)-2-(2,6-dioxopiperidin-3-yl)-6-fluoroisoindoline-1,3-dione;
4-(3,6-diazabicyclo[3.1.1]heptan-3-yl)-2-(2,6-dioxopiperidin-3-yl)-7-fluoroisoindoline-1,3-dione;
7-(3,6-diazabicyclo[3.1.1]heptan-3-yl)-2-(2,6-dioxopiperidin-3-yl)-4,5-difluoroisoindoline-1,3-dione;
4-(3,6-diazabicyclo[3.1.1]heptan-3-yl)-2-(2,6-dioxopiperidin-3-yl)-5,6-difluoroisoindoline-1,3-dione;
4-(3,6-diazabicyclo[3.1.1]heptan-3-yl)-2-(2,6-dioxopiperidin-3-yl)-5,7-difluoroisoindoline-1,3-dione;
4-(3,6-diazabicyclo[3.1.1]heptan-3-yl)-2-(2,6-dioxopiperidin-3-yl)-5,6,7-trifluoroisoindoline-1,3-dione;
4-((1R,5S)-3,8-diazabicyclo[3.2.1]octan-8-yl)-2-(2,6-dioxopiperidin-3-yl)-5-fluoroisoindoline-1,3-dione;
4-((1R,5S)-3,8-diazabicyclo[3.2.1]octan-8-yl)-2-(2,6-dioxopiperidin-3-yl)-6-fluoroisoindoline-1,3-dione;
4-((1R,5S)-3,8-diazabicyclo[3.2.1]octan-8-yl)-2-(2,6-dioxopiperidin-3-yl)-7-fluoroisoindoline-1,3-dione;
7-((1R,5S)-3,8-diazabicyclo[3.2.1]octan-8-yl)-2-(2,6-dioxopiperidin-3-yl)-4,5-difluoroisoindoline-1,3-dione;
4-((1R,5S)-3,8-diazabicyclo[3.2.1]octan-8-yl)-2-(2,6-dioxopiperidin-3-yl)-5,6-difluoroisoindoline-1,3-dione;
4-((1R,5S)-3,8-diazabicyclo[3.2.1]octan-8-yl)-2-(2,6-dioxopiperidin-3-yl)-5,7-difluoroisoindoline-1,3-dione;
4-((1R,5S)-3,8-diazabicyclo[3.2.1]octan-8-yl)-2-(2,6-dioxopiperidin-3-yl)-5,6,7-trifluoroisoindoline-1,3-dione;
4-((1R,5S)-3,8-diazabicyclo[3.2.1]octan-3-yl)-2-(2,6-dioxopiperidin-3-yl)-5-fluoroisoindoline-1,3-dione;
4-((1R,5S)-3,8-diazabicyclo[3.2.1]octan-3-yl)-2-(2,6-dioxopiperidin-3-yl)-6-fluoroisoindoline-1,3-dione;
4-((1R,5S)-3,8-diazabicyclo[3.2.1]octan-3-yl)-2-(2,6-dioxopiperidin-3-yl)-7-fluoroisoindoline-1,3-dione;
7-((1R,5S)-3,8-diazabicyclo[3.2.1]octan-3-yl)-2-(2,6-dioxopiperidin-3-yl)-4,5-difluoroisoindoline-1,3-dione;
4-((1R,5S)-3,8-diazabicyclo[3.2.1]octan-3-yl)-2-(2,6-dioxopiperidin-3-yl)-5,6-difluoroisoindoline-1,3-dione;
4-((1R,5S)-3,8-diazabicyclo[3.2.1]octan-3-yl)-2-(2,6-dioxopiperidin-3-yl)-5,7-difluoroisoindoline-1,3-dione;
4-((1R,5S)-3,8-diazabicyclo[3.2.1]octan-3-yl)-2-(2,6-dioxopiperidin-3-yl)-5,6,7-trifluoroisoindoline-1,3-dione;
4-(2,5-diazabicyclo[2.2.1]heptan-2-yl)-2-(2,6-dioxopiperidin-3-yl)-5-fluoroisoindoline-1,3-dione;
4-(2,5-diazabicyclo[2.2.1]heptan-2-yl)-2-(2,6-dioxopiperidin-3-yl)-6-fluoroisoindoline-1,3-dione;
4-(2,5-diazabicyclo[2.2.1]heptan-2-yl)-2-(2,6-dioxopiperidin-3-yl)-7-fluoroisoindoline-1,3-dione;
7-(2,5-diazabicyclo[2.2.1]heptan-2-yl)-2-(2,6-dioxopiperidin-3-yl)-4,5-difluoroisoindoline-1,3-dione;
4-(2,5-diazabicyclo[2.2.1]heptan-2-yl)-2-(2,6-dioxopiperidin-3-yl)-5,6-difluoroisoindoline-1,3-dione;
4-(2,5-diazabicyclo[2.2.1]heptan-2-yl)-2-(2,6-dioxopiperidin-3-yl)-5,7-difluoroisoindoline-1,3-dione;
4-(2,5-diazabicyclo[2.2.1]heptan-2-yl)-2-(2,6-dioxopiperidin-3-yl)-5,6,7-trifluoroisoindoline-1,3-dione;
4-(2,5-diazabicyclo[2.2.2]octan-2-yl)-2-(2,6-dioxopiperidin-3-yl)-5-fluoroisoindoline-1,3-dione;
4-(2,5-diazabicyclo[2.2.2]octan-2-yl)-2-(2,6-dioxopiperidin-3-yl)-6-fluoroisoindoline-1,3-dione;
4-(2,5-diazabicyclo[2.2.2]octan-2-yl)-2-(2,6-dioxopiperidin-3-yl)-7-fluoroisoindoline-1,3-dione;
7-(2,5-diazabicyclo[2.2.2]octan-2-yl)-2-(2,6-dioxopiperidin-3-yl)-4,5-difluoroisoindoline-1,3-dione;
4-(2,5-diazabicyclo[2.2.2]octan-2-yl)-2-(2,6-dioxopiperidin-3-yl)-5,6-difluoroisoindoline-1,3-dione;
4-(2,5-diazabicyclo[2.2.2]octan-2-yl)-2-(2,6-dioxopiperidin-3-yl)-5,7-difluoroisoindoline-1,3-dione;
4-(2,5-diazabicyclo[2.2.2]octan-2-yl)-2-(2,6-dioxopiperidin-3-yl)-5,6,7-trifluoroisoindoline-1,3-dione;
4-(2,6-dimethylpiperazin-1-yl)-2-(2,6-dioxopiperidin-3-yl)-5-fluoroisoindoline-1,3-dione;
4-(2,6-dimethylpiperazin-1-yl)-2-(2,6-dioxopiperidin-3-yl)-6-fluoroisoindoline-1,3-dione;
4-(2,6-dimethylpiperazin-1-yl)-2-(2,6-dioxopiperidin-3-yl)-7-fluoroisoindoline-1,3-dione;
7-(2,6-dimethylpiperazin-1-yl)-2-(2,6-dioxopiperidin-3-yl)-4,5-difluoroisoindoline-1,3-dione;
4-(2,6-dimethylpiperazin-1-yl)-2-(2,6-dioxopiperidin-3-yl)-5,6-difluoroisoindoline-1,3-dione;
4-(2,6-dimethylpiperazin-1-yl)-2-(2,6-dioxopiperidin-3-yl)-5,7-difluoroisoindoline-1,3-dione;
4-(2,6-dimethylpiperazin-1-yl)-2-(2,6-dioxopiperidin-3-yl)-5,6,7-trifluoroisoindoline-1,3-dione;
2-(2,6-dioxopiperidin-3-yl)-5-fluoro-4-(piperazin-1-yl-2,2,3,3,5,5,6,6-d₈)isoindoline-1,3-dione;
2-(2,6-dioxopiperidin-3-yl)-6-fluoro-4-(piperazin-1-yl-2,2,3,3,5,5,6,6-d₈)isoindoline-1,3-dione;
2-(2,6-dioxopiperidin-3-yl)-4-fluoro-7-(piperazin-1-yl-2,2,3,3,5,5,6,6-d₈)isoindoline-1,3-dione;
2-(2,6-dioxopiperidin-3-yl)-4,5-difluoro-7-(piperazin-1-yl-2,2,3,3,5,5,6,6-d₈)isoindoline-1,3-dione;
2-(2,6-dioxopiperidin-3-yl)-5,7-difluoro-4-(piperazin-1-yl-2,2,3,3,5,5,6,6-d₈)isoindoline-1,3-dione;
2-(2,6-dioxopiperidin-3-yl)-5,6-difluoro-4-(piperazin-1-yl-2,2,3,3,5,5,6,6-d₈)isoindoline-1,3-dione;
2-(2,6-dioxopiperidin-3-yl)-4,5,6-trifluoro-7-(piperazin-1-yl-2,2,3,3,5,5,6,6-d₈)isoindoline-1,3-dione;
2-(2,6-dioxopiperidin-3-yl)-5-fluoro-4-(piperidin-4-yl)isoindoline-1,3-dione;
2-(2,6-dioxopiperidin-3-yl)-4-fluoro-7-(piperidin-4-yl)isoindoline-1,3-dione;
2-(2,6-dioxopiperidin-3-yl)-4,5-difluoro-7-(piperidin-4-yl)isoindoline-1,3-dione;
2-(2,6-dioxopiperidin-3-yl)-5,6-difluoro-4-(piperidin-4-yl)isoindoline-1,3-dione;
2-(2,6-dioxopiperidin-3-yl)-5,7-difluoro-4-(piperidin-4-yl)isoindoline-1,3-dione;
2-(2,6-dioxopiperidin-3-yl)-4,5,6-trifluoro-7-(piperidin-4-yl)isoindoline-1,3-dione;
4-(3-azabicyclo[3.1.1]heptan-6-yl)-2-(2,6-dioxopiperidin-3-yl)-5-fluoroisoindoline-1,3-dione;
4-(3-azabicyclo[3.1.1]heptan-6-yl)-2-(2,6-dioxopiperidin-3-yl)-6-fluoroisoindoline-1,3-dione;
4-(3-azabicyclo[3.1.1]heptan-6-yl)-2-(2,6-dioxopiperidin-3-yl)-7-fluoroisoindoline-1,3-dione;
7-(3-azabicyclo[3.1.1]heptan-6-yl)-2-(2,6-dioxopiperidin-3-yl)-4,5-difluoroisoindoline-1,3-dione;
4-(3-azabicyclo[3.1.1]heptan-6-yl)-2-(2,6-dioxopiperidin-3-yl)-5,6-difluoroisoindoline-1,3-dione;
4-(3-azabicyclo[3.1.1]heptan-6-yl)-2-(2,6-dioxopiperidin-3-yl)-5,7-difluoroisoindoline-1,3-dione;
4-(3-azabicyclo[3.1.1]heptan-6-yl)-2-(2,6-dioxopiperidin-3-yl)-5,6,7-trifluoroisoindoline-1,3-dione;
4-(6-azabicyclo[3.1.1]heptan-3-yl)-2-(2,6-dioxopiperidin-3-yl)-5-fluoroisoindoline-1,3-dione;
4-(6-azabicyclo[3.1.1]heptan-3-yl)-2-(2,6-dioxopiperidin-3-yl)-6-fluoroisoindoline-1,3-dione;
4-(6-azabicyclo[3.1.1]heptan-3-yl)-2-(2,6-dioxopiperidin-3-yl)-7-fluoroisoindoline-1,3-dione;
7-(6-azabicyclo[3.1.1]heptan-3-yl)-2-(2,6-dioxopiperidin-3-yl)-4,5-difluoroisoindoline-1,3-dione;
4-(6-azabicyclo[3.1.1]heptan-3-yl)-2-(2,6-dioxopiperidin-3-yl)-5,6-difluoroisoindoline-1,3-dione;
4-(6-azabicyclo[3.1.1]heptan-3-yl)-2-(2,6-dioxopiperidin-3-yl)-5,7-difluoroisoindoline-1,3-dione;
4-(6-azabicyclo[3.1.1]heptan-3-yl)-2-(2,6-dioxopiperidin-3-yl)-5,6,7-trifluoroisoindoline-1,3-dione;
4-((1R,5S,8r)-3-azabicyclo[3.2.1]octan-8-yl)-2-(2,6-dioxopiperidin-3-yl)-5-fluoroisoindoline-1,3-dione;
4-((1R,5S,8r)-3-azabicyclo[3.2.1]octan-8-yl)-2-(2,6-dioxopiperidin-3-yl)-6-fluoroisoindoline-1,3-dione;
4-((1R,5S,8r)-3-azabicyclo[3.2.1]octan-8-yl)-2-(2,6-dioxopiperidin-3-yl)-7-fluoroisoindoline-1,3-dione;
7-((1R,5S,8r)-3-azabicyclo[3.2.1]octan-8-yl)-2-(2,6-dioxopiperidin-3-yl)-4,5-difluoroisoindoline-1,3-dione;
4-((1R,5S,8r)-3-azabicyclo[3.2.1]octan-8-yl)-2-(2,6-dioxopiperidin-3-yl)-5,6-difluoroisoindoline-1,3-dione;
4-((1R,5S,8r)-3-azabicyclo[3.2.1]octan-8-yl)-2-(2,6-dioxopiperidin-3-yl)-5,7-difluoroisoindoline-1,3-dione;
4-((1R,5S,8r)-3-azabicyclo[3.2.1]octan-8-yl)-2-(2,6-dioxopiperidin-3-yl)-5,6,7-trifluoroisoindoline-1,3-dione;
4-(3,5-dimethylpiperidin-4-yl)-2-(2,6-dioxopiperidin-3-yl)-5-fluoroisoindoline-1,3-dione;
4-(3,5-dimethylpiperidin-4-yl)-2-(2,6-dioxopiperidin-3-yl)-6-fluoroisoindoline-1,3-dione;
4-(3,5-dimethylpiperidin-4-yl)-2-(2,6-dioxopiperidin-3-yl)-7-fluoroisoindoline-1,3-dione;
7-(3,5-dimethylpiperidin-4-yl)-2-(2,6-dioxopiperidin-3-yl)-4,5-difluoroisoindoline-1,3-dione;
4-(3,5-dimethylpiperidin-4-yl)-2-(2,6-dioxopiperidin-3-yl)-5,6-difluoroisoindoline-1,3-dione;
4-(3,5-dimethylpiperidin-4-yl)-2-(2,6-dioxopiperidin-3-yl)-5,7-difluoroisoindoline-1,3-dione;
4-(3,5-dimethylpiperidin-4-yl)-2-(2,6-dioxopiperidin-3-yl)-5,6,7-trifluoroisoindoline-1,3-dione;
3-(4-(3,6-diazabicyclo[3.1.1]heptan-6-yl)-1-oxoisoindolin-2-yl)piperidine-2,6-dione;
3-(4-(3,6-diazabicyclo[3.1.1]heptan-3-yl)-1-oxoisoindolin-2-yl)piperidine-2,6-dione;
3-(4-((1R,5S)-3,8-diazabicyclo[3.2.1]octan-8-yl)-1-oxoisoindolin-2-yl)piperidine-2,6-dione;
3-(4-(2,5-diazabicyclo[2.2.1]heptan-2-yl)-1-oxoisoindolin-2-yl)piperidine-2,6-dione;
3-(4-(2,5-diazabicyclo[2.2.2]octan-2-yl)-1-oxoisoindolin-2-yl)piperidine-2,6-dione;
3-(4-((1R,5S)-3,8-diazabicyclo[3.2.1]octan-3-yl)-1-oxoisoindolin-2-yl)piperidine-2,6-dione;
3-(1-oxo-4-(piperazin-1-yl-2,2,3,3,5,5,6,6-d₈)isoindolin-2-yl)piperidine-2,6-dione;
3-(5-fluoro-1-oxo-4-(piperazin-1-yl)isoindolin-2-yl)piperidine-2,6-dione;
3-(7-fluoro-1-oxo-4-(piperazin-1-yl)isoindolin-2-yl)piperidine-2,6-dione;
3-(6,7-difluoro-1-oxo-4-(piperazin-1-yl)isoindolin-2-yl)piperidine-2,6-dione;
3-(5,6-difluoro-1-oxo-4-(piperazin-1-yl)isoindolin-2-yl)piperidine-2,6-dione;
3-(5,7-difluoro-1-oxo-4-(piperazin-1-yl)isoindolin-2-yl)piperidine-2,6-dione;
3-(5,6,7-trifluoro-1-oxo-4-(piperazin-1-yl)isoindolin-2-yl)piperidine-2,6-dione;
3-(4-(3,6-diazabicyclo[3.1.1]heptan-6-yl)-5-fluoro-1-oxoisoindolin-2-yl)piperidine-2,6-dione;
3-(4-(3,6-diazabicyclo[3.1.1]heptan-6-yl)-6-fluoro-1-oxoisoindolin-2-yl)piperidine-2,6-dione;
3-(4-(3,6-diazabicyclo[3.1.1]heptan-6-yl)-7-fluoro-1-oxoisoindolin-2-yl)piperidine-2,6-dione;
3-(4-(3,6-diazabicyclo[3.1.1]heptan-6-yl)-6,7-difluoro-1-oxoisoindolin-2-yl)piperidine-2,6-dione;
3-(4-(3,6-diazabicyclo[3.1.1]heptan-6-yl)-5,6-difluoro-1-oxoisoindolin-2-yl)piperidine-2,6-dione;
3-(4-(3,6-diazabicyclo[3.1.1]heptan-6-yl)-5,7-difluoro-1-oxoisoindolin-2-yl)piperidine-2,6-dione;
3-(4-(3,6-diazabicyclo[3.1.1]heptan-6-yl)-5,6,7-trifluoro-1-oxoisoindolin-2-yl)piperidine-2,6-dione;
3-(4-(3,6-diazabicyclo[3.1.1]heptan-3-yl)-5-fluoro-1-oxoisoindolin-2-yl)piperidine-2,6-dione;
3-(4-(3,6-diazabicyclo[3.1.1]heptan-3-yl)-6-fluoro-1-oxoisoindolin-2-yl)piperidine-2,6-dione;
3-(4-(3,6-diazabicyclo[3.1.1]heptan-3-yl)-7-fluoro-1-oxoisoindolin-2-yl)piperidine-2,6-dione;
3-(4-(3,6-diazabicyclo[3.1.1]heptan-3-yl)-6,7-difluoro-1-oxoisoindolin-2-yl)piperidine-2,6-dione;
3-(4-(3,6-diazabicyclo[3.1.1]heptan-3-yl)-5,6-difluoro-1-oxoisoindolin-2-yl)piperidine-2,6-dione;
3-(4-(3,6-diazabicyclo[3.1.1]heptan-3-yl)-5,7-difluoro-1-oxoisoindolin-2-yl)piperidine-2,6-dione;
3-(4-(3,6-diazabicyclo[3.1.1]heptan-3-yl)-5,6,7-trifluoro-1-oxoisoindolin-2-yl)piperidine-2,6-dione;
3-(4-((1R,5S)-3,8-diazabicyclo[3.2.1]octan-8-yl)-5-fluoro-1-oxoisoindolin-2-yl)piperidine-2,6-dione;
3-(4-((1R,5S)-3,8-diazabicyclo[3.2.1]octan-8-yl)-6-fluoro-1-oxoisoindolin-2-yl)piperidine-2,6-dione;
3-(4-((1R,5S)-3,8-diazabicyclo[3.2.1]octan-8-yl)-7-fluoro-1-oxoisoindolin-2-yl)piperidine-2,6-dione;
3-(4-((1R,5S)-3,8-diazabicyclo[3.2.1]octan-8-yl)-6,7-difluoro-1-oxoisoindolin-2-yl)piperidine-2,6-dione;
3-(4-((1R,5S)-3,8-diazabicyclo[3.2.1]octan-8-yl)-5,6-difluoro-1-oxoisoindolin-2-yl)piperidine-2,6-dione;
3-(4-((1R,5S)-3,8-diazabicyclo[3.2.1]octan-8-yl)-5,7-difluoro-1-oxoisoindolin-2-yl)piperidine-2,6-dione;
3-(4-((1R,5S)-3,8-diazabicyclo[3.2.1]octan-8-yl)-5,6,7-trifluoro-1-oxoisoindolin-2-yl)piperidine-2,6-dione;
3-(4-((1R,5S)-3,8-diazabicyclo[3.2.1]octan-3-yl)-5-fluoro-1-oxoisoindolin-2-yl)piperidine-2,6-dione;
3-(4-((1R,5S)-3,8-diazabicyclo[3.2.1]octan-3-yl)-6-fluoro-1-oxoisoindolin-2-yl)piperidine-2,6-dione;
3-(4-((1R,5S)-3,8-diazabicyclo[3.2.1]octan-3-yl)-7-fluoro-1-oxoisoindolin-2-yl)piperidine-2,6-dione;
3-(4-((1R,5S)-3,8-diazabicyclo[3.2.1]octan-3-yl)-6,7-difluoro-1-oxoisoindolin-2-yl)piperidine-2,6-dione;
3-(4-((1R,5S)-3,8-diazabicyclo[3.2.1]octan-3-yl)-5,6-difluoro-1-oxoisoindolin-2-yl)piperidine-2,6-dione;
3-(4-((1R,5S)-3,8-diazabicyclo[3.2.1]octan-3-yl)-5,7-difluoro-1-oxoisoindolin-2-yl)piperidine-2,6-dione;
3-(4-((1R,5S)-3,8-diazabicyclo[3.2.1]octan-3-yl)-5,6,7-trifluoro-1-oxoisoindolin-2-yl)piperidine-2,6-dione;
3-(4-(2,5-diazabicyclo[2.2.1]heptan-2-yl)-5-fluoro-1-oxoisoindolin-2-yl)piperidine-2,6-dione;
3-(4-(2,5-diazabicyclo[2.2.1]heptan-2-yl)-6-fluoro-1-oxoisoindolin-2-yl)piperidine-2,6-dione;
3-(4-(2,5-diazabicyclo[2.2.1]heptan-2-yl)-7-fluoro-1-oxoisoindolin-2-yl)piperidine-2,6-dione;
3-(4-(2,5-diazabicyclo[2.2.1]heptan-2-yl)-6,7-difluoro-1-oxoisoindolin-2-yl)piperidine-2,6-dione;
3-(4-(2,5-diazabicyclo[2.2.1]heptan-2-yl)-5,6-difluoro-1-oxoisoindolin-2-yl)piperidine-2,6-dione;
3-(4-(2,5-diazabicyclo[2.2.1]heptan-2-yl)-5,7-difluoro-1-oxoisoindolin-2-yl)piperidine-2,6-dione;
3-(4-(2,5-diazabicyclo[2.2.1]heptan-2-yl)-5,6,7-trifluoro-1-oxoisoindolin-2-yl)piperidine-2,6-dione;
3-(4-(2,5-diazabicyclo[2.2.2]octan-2-yl)-5-fluoro-1-oxoisoindolin-2-yl)piperidine-2,6-dione;
3-(4-(2,5-diazabicyclo[2.2.2]octan-2-yl)-6-fluoro-1-oxoisoindolin-2-yl)piperidine-2,6-dione;
3-(4-(2,5-diazabicyclo[2.2.2]octan-2-yl)-7-fluoro-1-oxoisoindolin-2-yl)piperidine-2,6-dione;
3-(4-(2,5-diazabicyclo[2.2.2]octan-2-yl)-6,7-difluoro-1-oxoisoindolin-2-yl)piperidine-2,6-dione;
3-(4-(2,5-diazabicyclo[2.2.2]octan-2-yl)-5,6-difluoro-1-oxoisoindolin-2-yl)piperidine-2,6-dione;
3-(4-(2,5-diazabicyclo[2.2.2]octan-2-yl)-5,7-difluoro-1-oxoisoindolin-2-yl)piperidine-2,6-dione;
3-(4-(2,5-diazabicyclo[2.2.2]octan-2-yl)-5,6,7-trifluoro-1-oxoisoindolin-2-yl)piperidine-2,6-dione;
3-(4-(2,6-dimethylpiperazin-1-yl)-5-fluoro-1-oxoisoindolin-2-yl)piperidine-2,6-dione;
3-(4-(2,6-dimethylpiperazin-1-yl)-6-fluoro-1-oxoisoindolin-2-yl)piperidine-2,6-dione;
3-(4-(2,6-dimethylpiperazin-1-yl)-7-fluoro-1-oxoisoindolin-2-yl)piperidine-2,6-dione;
3-(4-(2,6-dimethylpiperazin-1-yl)-6,7-difluoro-1-oxoisoindolin-2-yl)piperidine-2,6-dione;
3-(4-(2,6-dimethylpiperazin-1-yl)-5,6-difluoro-1-oxoisoindolin-2-yl)piperidine-2,6-dione;
3-(4-(2,6-dimethylpiperazin-1-yl)-5,7-difluoro-1-oxoisoindolin-2-yl)piperidine-2,6-dione;
3-(4-(2,6-dimethylpiperazin-1-yl)-5,6,7-trifluoro-1-oxoisoindolin-2-yl)piperidine-2,6-dione;
3-(5-fluoro-1-oxo-4-(piperazin-1-yl-2,2,3,3,5,5,6,6-d₈)isoindolin-2-yl)piperidine-2,6-dione;
3-(6-fluoro-1-oxo-4-(piperazin-1-yl-2,2,3,3,5,5,6,6-d₈)isoindolin-2-yl)piperidine-2,6-dione;
3-(7-fluoro-1-oxo-4-(piperazin-1-yl-2,2,3,3,5,5,6,6-d₈)isoindolin-2-yl)piperidine-2,6-dione;
3-(6,7-difluoro-1-oxo-4-(piperazin-1-yl-2,2,3,3,5,5,6,6-d₈)isoindolin-2-yl)piperidine-2,6-dione;
3-(5,7-difluoro-1-oxo-4-(piperazin-1-yl-2,2,3,3,5,5,6,6-d₈)isoindolin-2-yl)piperidine-2,6-dione;
3-(5,6-difluoro-1-oxo-4-(piperazin-1-yl-2,2,3,3,5,5,6,6-d₈)isoindolin-2-yl)piperidine-2,6-dione;
3-(5,6,7-trifluoro-1-oxo-4-(piperazin-1-yl-2,2,3,3,5,5,6,6-d₈)isoindolin-2-yl)piperidine-2,6-dione;
3-(5-fluoro-1-oxo-4-(piperidin-4-yl)isoindolin-2-yl)piperidine-2,6-dione;
3-(7-fluoro-1-oxo-4-(piperidin-4-yl)isoindolin-2-yl)piperidine-2,6-dione;
3-(6,7-difluoro-1-oxo-4-(piperidin-4-yl)isoindolin-2-yl)piperidine-2,6-dione;
3-(5,6-difluoro-1-oxo-4-(piperidin-4-yl)isoindolin-2-yl)piperidine-2,6-dione;
3-(5,7-difluoro-1-oxo-4-(piperidin-4-yl)isoindolin-2-yl)piperidine-2,6-dione;
3-(5,6,7-trifluoro-1-oxo-4-(piperidin-4-yl)isoindolin-2-yl)piperidine-2,6-dione;
3-(4-(3-azabicyclo[3.1.1]heptan-6-yl)-5-fluoro-1-oxoisoindolin-2-yl)piperidine-2,6-dione;
3-(4-(3-azabicyclo[3.1.1]heptan-6-yl)-6-fluoro-1-oxoisoindolin-2-yl)piperidine-2,6-dione;
3-(4-(3-azabicyclo[3.1.1]heptan-6-yl)-7-fluoro-1-oxoisoindolin-2-yl)piperidine-2,6-dione;
3-(4-(3-azabicyclo[3.1.1]heptan-6-yl)-6,7-difluoro-1-oxoisoindolin-2-yl)piperidine-2,6-dione;
3-(4-(3-azabicyclo[3.1.1]heptan-6-yl)-5,6-difluoro-1-oxoisoindolin-2-yl)piperidine-2,6-dione;
3-(4-(3-azabicyclo[3.1.1]heptan-6-yl)-5,7-difluoro-1-oxoisoindolin-2-yl)piperidine-2,6-dione;
3-(4-(3-azabicyclo[3.1.1]heptan-6-yl)-5,6,7-trifluoro-1-oxoisoindolin-2-yl)piperidine-2,6-dione;
3-(4-(6-azabicyclo[3.1.1]heptan-3-yl)-5-fluoro-1-oxoisoindolin-2-yl)piperidine-2,6-dione;
3-(4-(6-azabicyclo[3.1.1]heptan-3-yl)-6-fluoro-1-oxoisoindolin-2-yl)piperidine-2,6-dione;
3-(4-(6-azabicyclo[3.1.1]heptan-3-yl)-7-fluoro-1-oxoisoindolin-2-yl)piperidine-2,6-dione;
3-(4-(6-azabicyclo[3.1.1]heptan-3-yl)-6,7-difluoro-1-oxoisoindolin-2-yl)piperidine-2,6-dione;
3-(4-(6-azabicyclo[3.1.1]heptan-3-yl)-5,6-difluoro-1-oxoisoindolin-2-yl)piperidine-2,6-dione;
3-(4-(6-azabicyclo[3.1.1]heptan-3-yl)-5,7-difluoro-1-oxoisoindolin-2-yl)piperidine-2,6-dione;
3-(4-(6-azabicyclo[3.1.1]heptan-3-yl)-5,6,7-trifluoro-1-oxoisoindolin-2-yl)piperidine-2,6-dione;
3-(4-((1R,5S,8r)-3-azabicyclo[3.2.1]octan-8-yl)-5-fluoro-1-oxoisoindolin-2-yl)piperidine-2,6-dione;
3-(4-((1R,5S,8r)-3-azabicyclo[3.2.1]octan-8-yl)-6-fluoro-1-oxoisoindolin-2-yl)piperidine-2,6-dione;
3-(4-((1R,5S,8r)-3-azabicyclo[3.2.1]octan-8-yl)-7-fluoro-1-oxoisoindolin-2-yl)piperidine-2,6-dione;
3-(4-((1R,5S,8r)-3-azabicyclo[3.2.1]octan-8-yl)-6,7-difluoro-1-oxoisoindolin-2-yl)piperidine-2,6-dione;
3-(4-((1R,5S,8r)-3-azabicyclo[3.2.1]octan-8-yl)-5,6-difluoro-1-oxoisoindolin-2-yl)piperidine-2,6-dione;
3-(4-((1R,5S,8r)-3-azabicyclo[3.2.1]octan-8-yl)-5,7-difluoro-1-oxoisoindolin-2-yl)piperidine-2,6-dione;
3-(4-((1R,5S,8r)-3-azabicyclo[3.2.1]octan-8-yl)-5,6,7-trifluoro-1-oxoisoindolin-2-yl)piperidine-2,6-dione;
3-(4-(3,5-dimethylpiperidin-4-yl)-5-fluoro-1-oxoisoindolin-2-yl)piperidine-2,6-dione;
3-(4-(3,5-dimethylpiperidin-4-yl)-6-fluoro-1-oxoisoindolin-2-yl)piperidine-2,6-dione;
3-(4-(3,5-dimethylpiperidin-4-yl)-7-fluoro-1-oxoisoindolin-2-yl)piperidine-2,6-dione;
3-(4-(3,5-dimethylpiperidin-4-yl)-6,7-difluoro-1-oxoisoindolin-2-yl)piperidine-2,6-dione;
3-(4-(3,5-dimethylpiperidin-4-yl)-5,6-difluoro-1-oxoisoindolin-2-yl)piperidine-2,6-dione;
3-(4-(3,5-dimethylpiperidin-4-yl)-5,7-difluoro-1-oxoisoindolin-2-yl)piperidine-2,6-dione; and
3-(4-(3,5-dimethylpiperidin-4-yl)-5,6,7-trifluoro-1-oxoisoindolin-2-yl)piperidine-2,6-dione.

9. A compound of Formula (II) 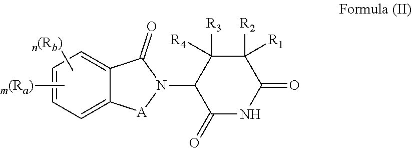
or salts, enantiomers, stereoisomers, solvates, isotopically enriched analogs, prodrugs, or polymorphs thereof,
wherein A represents CO, CH₂ or CD₂;
R₁, R₂, R₃ and R₄ are the same or different and each independently represent hydrogen, deuterium, halogen, optionally deuterated C₁₋₆ alkyl, optionally deuterated C₁₋₆ alkoxy, or halogenated C₁₋₆ alkyl;
(R_{b})ₙ indicates that benzene ring is substituted with n R_{b} substituents, and each R_{b} are the same or different and each independently represent hydrogen, deuterium, halogen, hydroxy, mercapto, nitro, amino, cyano, optionally deuterated C₁₋₆ alkyl, optionally deuterated C₁₋₆ alkoxy, halogenated C₁₋₆ alkyl, C₂₋₆ alkenyl or C₂₋₆ alkynyl;
(Rₐ)ₘ indicates that the benzene ring is substituted with m Rₐ substituents, and each Rₐ are the same or different and each independently represents the structure of the following formula:
W-LIN—U₂—R_{c}—U₁—,
wherein
U₁ and U₂ are the same or different and each independently represent a bond, N(R_{d}) or O, wherein R_{d} represents H or optionally deuterated C₁₋₆ alkyl;
R_{c} represents optionally substituted nitrogen-containing monocyclic heterocyclylene or optionally substituted nitrogen-containing bridged heterocyclylene;
W represents hydrogen, optionally substituted nitrogen-containing monocyclic heterocyclyl, optionally substituted nitrogen-containing bridged heterocyclyl, optionally substituted cycloalkyl, optionally substituted heteroaryl, optionally substituted aryl, C₂₋₆ alkenyl, C₂₋₆ alkynyl, a leaving group, CHO, COOH, NH₂, hydroxy or mercapto; and
wherein in case that the compound of Formula (II) is partially or fully deuterated:
when W represents optionally substituted nitrogen-containing monocyclic heterocyclyl, optionally substituted nitrogen-containing bridged heterocyclyl, optionally substituted cycloalkyl, optionally substituted heteroaryl or optionally substituted aryl, LIN represents a bond, optionally substituted methylene, or optionally substituted linear or branched C₂₋₃₀ alkylene, wherein one or more groups Rₑ and/or one or more groups R_{f} and/or any combination of one or more groups Rₑ and R_{f} are optionally inserted into the backbone carbon chain of the linear or branched C₂₋₃₀ alkylene group, wherein carbon-carbon bond between one or more pairs of adjacent carbon atoms in the backbone carbon chain is interrupted by the group Rₑ, R_{f}, or a combination of Rₑ and R_{f}; wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl, and in case that two or more groups Rₑ are inserted into the backbone carbon chain of the linear or branched C₂₋₃₀ alkylene group, the two or more groups Rₑ are not directly connected to each other; and wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano or any combination thereof; or
when W represents hydrogen, C₂₋₆ alkenyl, C₂₋₆ alkynyl, a leaving group, CHO, COOH, NH₂, hydroxy or mercapto, LIN represents optionally substituted methylene, or optionally substituted linear or branched C₂₋₃₀ alkylene, wherein one or more groups Rₑ and/or one or more groups R_{f} and/or any combination of one or more groups Rₑ and R_{f} are optionally inserted into the backbone carbon chain of the linear or branched C₂₋₃₀ alkylene group, wherein carbon-carbon bond between one or more pairs of adjacent carbon atoms in the backbone carbon chain is interrupted by the group Rₑ, R_{f}, or a combination of Rₑ and R_{f}; wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl, and in case that two or more groups Rₑ are inserted into the backbone carbon chain of the linear or branched C₂₋₃₀ alkylene group, the two or more groups Rₑ are not directly connected to each other; and wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano or any combination thereof; or
in case that the compound of Formula (II) is not deuterated, LIN represents optionally substituted methylene, and W represents optionally substituted nitrogen-containing monocyclic heterocyclyl or optionally substituted nitrogen-containing bridged heterocyclyl; and
m represents an integer of 1, 2, 3 or 4, n represents an integer of 0, 1, 2 or 3, and the sum of m and n is an integer of 1, 2, 3 or 4;
with the proviso that when the compound of Formula (II) is not deuterated, the following compounds are excluded:
3-(4,6-difluoro-1-oxo-5-(4-(piperidin-4-ylmethyl)piperazin-1-yl)isoindolin-2-yl)piperidine-2,6-dione;
2-(2,6-dioxopiperidin-3-yl)-5-(4-(piperidin-4-ylmethyl)piperazin-1-yl)isoindoline-1,3-dione;
3-(4-chloro-1-oxo-5-(4-(piperidin-4-ylmethyl)piperidin-1-yl)isoindolin-2-yl)piperidine-2,6-dione;
5-(4-(azetidin-3-ylmethyl)piperazin-1-yl)-2-(2,6-dioxopiperidin-3-yl)isoindoline-1,3-dione; and
2-(2,6-dioxopiperidin-3-yl)-5-(3-(piperazin-1-ylmethyl)azetidin-1-yl)isoindoline-1,3-dione.

10. The compound of Formula (II) or salts, enantiomers, stereoisomers, solvates, isotopically enriched analogs, prodrugs, or polymorphs thereof as claimed in claim 9, wherein the compound of Formula (II) is also of Formula (II-1) or Formula (II-2): 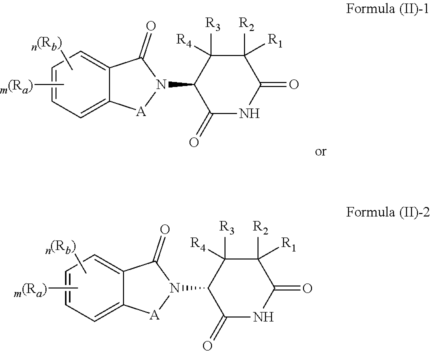
wherein A, R₁, R₂, R₃, R₄, Rₐ, R_{b}, m, and n are as defined in claim 9.

11. The compound of Formula (II) or salts, enantiomers, stereoisomers, solvates, isotopically enriched analogs, prodrugs, or polymorphs thereof as claimed in claim 9, wherein
when the compound of Formula (II) is partially deuterated (for example, carrying 1 to 20 deuterium atoms) or fully deuterated, and W represents optionally substituted nitrogen-containing monocyclic heterocyclyl, optionally substituted nitrogen-containing bridged heterocyclyl, optionally substituted cycloalkyl, optionally substituted heteroaryl or optionally substituted aryl, LIN represents a bond, optionally substituted methylene, or optionally substituted linear or branched C₂₋₃₀ alkylene, wherein one or more groups Rₑ and/or one or more groups R_{f} and/or any combination of one or more groups Rₑ and R_{f} are optionally inserted into the backbone carbon chain of the linear or branched C₂₋₃₀ alkylene group, wherein carbon-carbon bond between one or more pairs of adjacent carbon atoms in the backbone carbon chain is interrupted by the group Rₑ, R_{f}, or a combination of Rₑ and R_{f}; each Rₑ are selected from the group consisting of O, N(R_{g}), C(O), C(O)O, S(O), S(O)₂, S(O)₂NH, NHS(O)₂, OC(O), C(O)N(R_{g}), N(R_{g})C(O), or N(R_{g})C(O)N(R_{g}), where each R_{g} independently represent H or C₁₋₆ alkyl, and in case that two or more groups Rₑ are inserted into the backbone carbon chain of the linear or branched C₂₋₃₀ alkylene group, the two or more groups Rₑ are not directly connected to each other; and each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl, optionally deuterated C₃₋₆ cycloalkyl, hydroxyl, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano or any combination thereof; or
when the compound of Formula (II) is partially deuterated (for example, carrying 1 to 20 deuterium atom) or fully deuterated, and W represents hydrogen, C₂₋₆ alkenyl, C₂₋₆ alkynyl, a leaving group, CHO, COOH, NH₂, hydroxy or mercapto, LIN represents optionally substituted methylene or optionally substituted linear or branched C₂₋₃₀ alkylene, wherein one or more groups Rₑ and/or one or more groups R_{f} and/or any combination of one or more groups Rₑ and R_{f} are optionally inserted into the backbone carbon chain of the linear or branched C₂₋₃₀ alkylene group, wherein carbon-carbon bond between one or more pairs of adjacent carbon atoms in the backbone carbon chain is interrupted by the group Rₑ, R_{f}, or a combination of Rₑ and R_{f}; each Rₑ are selected from the group consisting of O, N(R_{g}), C(O), C(O)O, S(O), S(O)₂, S(O)₂NH, NHS(O)₂, OC(O), C(O)N(R_{g}), N(R_{g})C(O), or N(R_{g})C(O)N(R_{g}), where each R_{g} independently represent H or C₁₋₆ alkyl, and in case that two or more groups Rₑ are inserted into the backbone carbon chain of the linear or branched C₂₋₃₀ alkylene group, the two or more groups Rₑ are not directly connected to each other; and each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl, optionally deuterated C₃₋₆ cycloalkyl, hydroxyl, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano or any combination thereof; or
when the compound of Formula (II) is not deuterated, LIN represents optionally substituted methylene, and W represents optionally substituted nitrogen-containing monocyclic heterocyclyl or optionally substituted nitrogen-containing bridged heterocyclyl;
wherein the methylene and the linear or branched C₂₋₃₀ alkylene are optionally substituted with one or more substituents selected from the group consisting of deuterium, hydroxy, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof.

12. The compound of Formula (II) or salts, enantiomers, stereoisomers, solvates, isotopically enriched analogs, prodrugs, or polymorphs thereof as claimed in claim 9, wherein (i) R_{c} represents the following groups:
monocyclic C₃₋₁₀ heterocyclylene group containing one nitrogen atom and optionally a heteroatom selected from nitrogen, oxygen, and sulfur, e.g., piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene or diazacyclooctylene; or
C₅₋₂₀ bridged heterocyclylene group containing one nitrogen atom and optionally a heteroatom selected from nitrogen, oxygen, and sulfur, e.g., azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene or diazabicyclo[2.2.2]octanylene, such as the following groups: 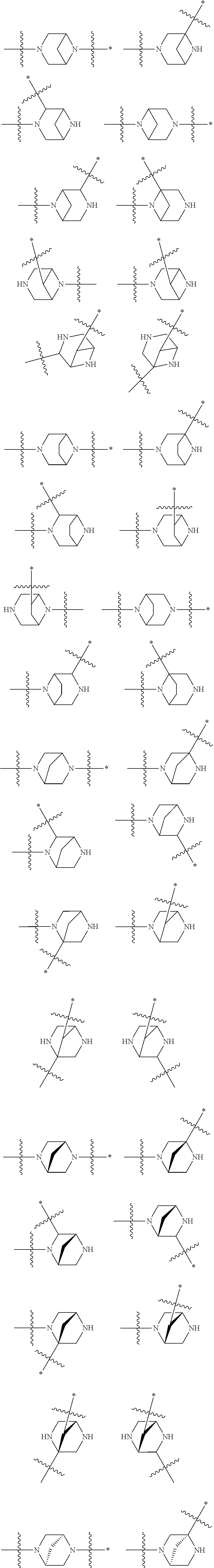 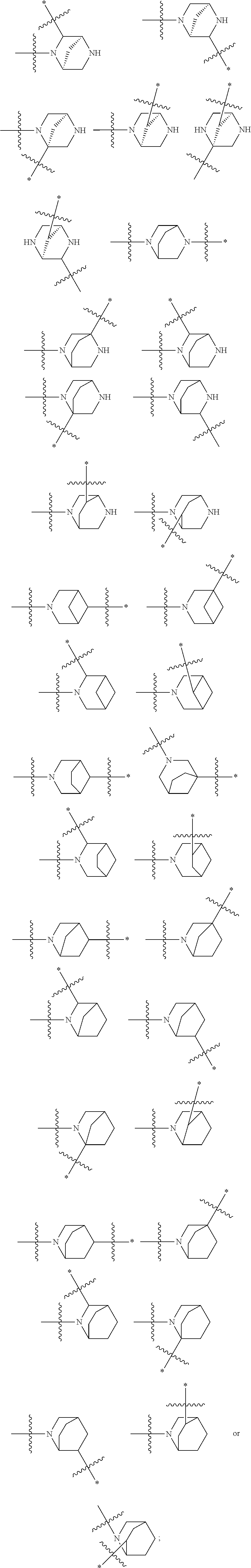
wherein symbol * indicates the point of attachment to U₁,
wherein the monocyclic C₃₋₁₀ heterocyclylene and the C₅₋₂₀ bridged heterocyclylene group are each independently optionally substituted with one or more substituents selected from the group consisting of deuterium, hydroxy, amino, mercapto, nitro, halogen, cyano, CHO, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof;
and/or
(ii) W represents the following groups:
monocyclic C₃₋₁₀ heterocyclyl group containing one nitrogen atom and optionally a heteroatom selected from nitrogen, oxygen, and sulfur, e.g., piperidinyl, piperazinyl, morpholinyl, azetidinyl, pyrrolidinyl, imidazolidyl, pyrazolidyl, oxazolidinyl, thiazolidinyl, thiomorpholinyl, azepanyl, diazacycloheptanyl, azacyclooctyl or diazacyclooctyl; or
C₅₋₂₀ bridged heterocyclyl group containing one nitrogen atom and optionally a heteroatom selected from nitrogen, oxygen, and sulfur, e.g., azabicyclo[3.1.1]heptanyl, azabicyclo[2.2.1]heptanyl, azabicyclo[3.2.1]octanyl, azabicyclo[2.2.2]octanyl, diazabicyclo[3.1.1]heptanyl, diazabicyclo[2.2.1]heptanyl, diazabicyclo[3.2.1]octanyl or diazabicyclo[2.2.2]octanyl, such as the following groups: 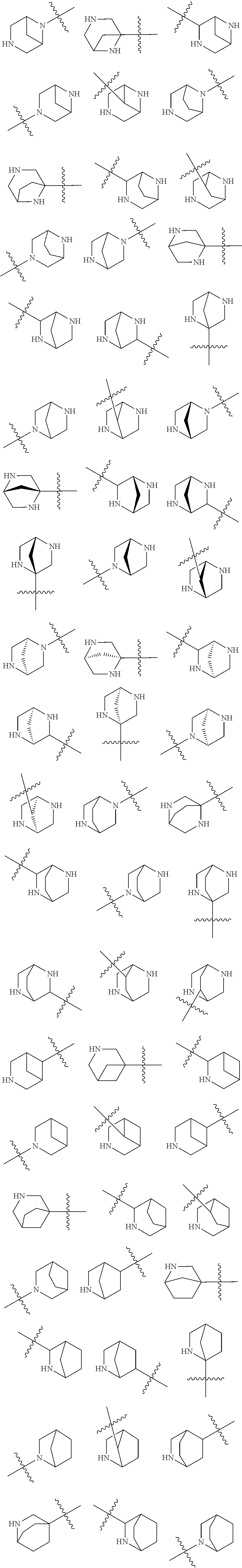 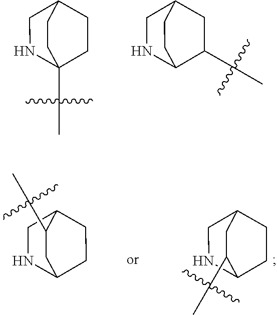 [SMILES: *C12CCC(CC1)CN2 ; *C1CC2CCC1NC2 ; *C1CC2CCC1CN2 ; "or" ; *C1CC2CCC1NC2 ; ";"]
or
saturated or partially unsaturated monocyclic or bicyclic or polycyclic C₃₋₂₀ cycloalkyl, e.g., cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, C₇₋₁₁ spiro-cycloalkyl, adamantanyl, noradamantanyl, bornyl, bicyclo[2.2.1]heptyl or bicyclo[2.2.1]heptenyl; or
heteroaryl, e.g., 5- to 20-membered monocyclic or bicyclic aromatic ring group containing one or more heteroatoms independently selected from the group consisting of oxygen, nitrogen, and sulfur, e.g., furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzisoxazolyl, benzothiazolyl, benzisothiazolyl, benzotriazolyl, benzo[2,1,3]oxadiazolyl, benzo[2,1,3]thiadiazolyl, benzo[1,2,3]thiadiazolyl, quinolinyl, isoquinolinyl, naphthyridinyl, cinnolinyl, quinazolinyl, quinoxalinyl, phthalazinyl, pyrazolo[1,5-a]pyridyl, pyrazolo[1,5-a]pyrimidinyl, imidazo[1,2-a]pyridyl, 1H-pyrrolo[3,2-b]pyridyl, 1H-pyrrolo[2,3-b]pyridyl, 4H-fluoro[3,2-b]pyrrolyl, pyrrolo[2,1-b]thiazolyl or imidazo[2,1-b]thiazolyl; or
C₅₋₁₄ aryl optionally containing one or more fused rings, e.g., phenyl, naphthyl or fluorenyl; and
wherein the C₃₋₁₀ heterocyclyl, C₅₋₂₀ bridged heterocyclyl, C₃₋₂₀ cycloalkyl, heteroaryl and C₅₋₁₄ aryl are each independently optionally substituted with one or more substituents selected from the group consisting of deuterium, hydroxy, amino, mercapto, nitro, halogen, cyano, CHO, COOH, N₃, methanesulfonyloxy, trifluoromethanesulfonyloxy, p-toluenesulfonyloxy, C₁₋₆ alkyl, deuterated C₁₋₆ alkyl, halogenated C₁₋₆ alkyl, optionally substituted C₃₋₈ cycloalkyl (e.g., substituted with one or more substituents selected from the group consisting of deuterium, halogen, amino, hydroxy, mercapto, cyano, CHO, COOH, N₃, methanesulfonyloxy, trifluoromethanesulfonyloxy and p-toluenesulfonyloxy), optionally substituted 4- to 9-membered heterocyclyl (e.g., substituted with one or more substituents selected from the group consisting of deuterium, halogen, amino, hydroxy, mercapto, cyano, CHO, COOH, N₃, methanesulfonyloxy, trifluoromethanesulfonyloxy and p-toluenesulfonyloxy), C₁₋₆ alkoxy, deuterated C₁₋₆ alkyl-O—, C₁₋₆ alkyl-NH—, deuterated C₁₋₆ alkyl-NH—, NH₂—C₁₋₆ alkylene, C₁₋₆ alkyl-NHC(O)—, deuterated C₁₋₆ alkyl-NHC(O)—, C₁₋₆ alkyl-C(O)NH—, deuterated C₁₋₆ alkyl-C(O)NH— or any combination thereof;
and/or
(iii) LIN represents the structure of the following formula:
#-C₁₋₃₀ alkylene-;
#-(C(R^{a1})(R^{a2}))ₙ₁—(Rₑ(C(R^{a3})(R^{a4}))ₙ₂)ₘ₁—;
#-(C(R^{a1})(R^{a2}))ₙ₁—(Rₑ(C(R^{a3})(R^{a4}))ₙ₂)ₘ₁—(Rₑ(C(R^{a5})(R^{a6}))ₙ₃)ₘ₂—;
#-(C(R^{a1})(R^{a2}))ₙ₁—(Rₑ(C(R^{a3})(R^{a4}))ₙ₂)ₘ₁—(Rₑ(C(R^{a5})(R^{a6}))ₙ₃)ₘ₂—(Rₑ(C(R^{a7})(R^{a8}))ₙ₄)ₘ₃—;
#-(C(R^{a1})(R^{a2}))ₙ₁—(R_{f}(C(R^{a3})(R^{a4}))ₙ₂)ₘ₁—;
#-(C(R^{a1})(R^{a2}))ₙ₁—(R_{f}(C(R^{a3})(R^{a4}))ₙ₂)ₘ₁—(R_{f}(C(R^{a5})(R^{a6}))ₙ₃)ₘ₂—;
#-(C(R^{a1})(R^{a2}))ₙ₁—(R_{f}(C(R^{a3})(R^{a4}))ₙ₂)ₘ₁—(R_{f}(C(R^{a5})(R^{a6}))ₙ₃)ₘ₂—(R_{f}(C(R^{a7})(R^{a8}))ₙ₄)ₘ₃—;
#-(C(R^{a1})(R^{a2}))ₙ₁—(Rₑ—R_{f}—(C(R^{a3})(R^{a4}))ₙ₂)ₘ₁—;
#-(C(R^{a1})(R^{a2}))ₙ₁—(Rₑ(C(R^{a3})(R^{a4}))ₙ₂)ₘ₁—(R_{f}(C(R^{a5})(R^{a6}))ₙ₃)ₘ₂—;
#-(C(R^{a1})(R^{a2}))ₙ₁—(R_{f}—Rₑ—(C(R^{a3})(R^{a4}))ₙ₂)ₘ₁—; or
#-(C(R^{a1})(R^{a2}))ₙ₁—(R_{f}(C(R^{a3})(R^{a4}))ₙ₂)ₘ₁—(Rₑ(C(R^{a5})(R^{a6}))ₙ₃)ₘ₂—;
wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl;
wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano, or any combination thereof;
a hydrogen atom of one or more CH₂ groups of the C₁₋₃₀ alkylene group is optionally replaced with a substituent selected from the group consisting of deuterium, hydroxy, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH—, or any combination thereof;
R^{a1}, Rₐ₂, R^{3a}, R^{a4}, R^{a5}, R^{a6}, R^{a7}, and R^{a8} each independently represent H, deuterium, hydroxy, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH—, or any combination thereof;
symbol # indicates the point of attachment to U₂; and
n1, n2, n3, n4, m1, m2, and m3 each independently represent an integer of 1, 2, 3, 4, 5, 6, 7, 8, 9, 10, 11, 12, 13, 14, or 15.

13. The compound of Formula (II) or salts, enantiomers, stereoisomers, solvates, isotopically enriched analogs, prodrugs, or polymorphs thereof as claimed in claim 9, wherein R_{c} represents the following groups: 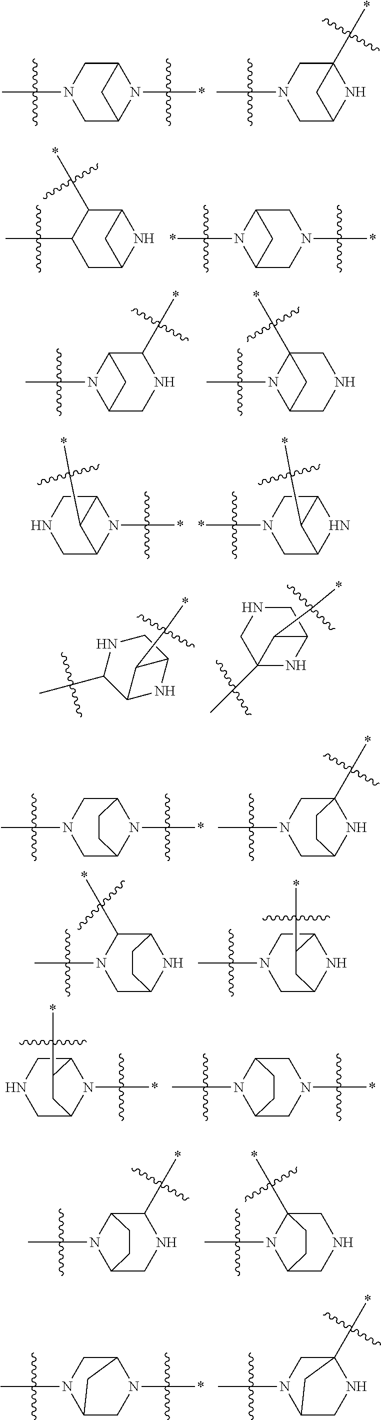 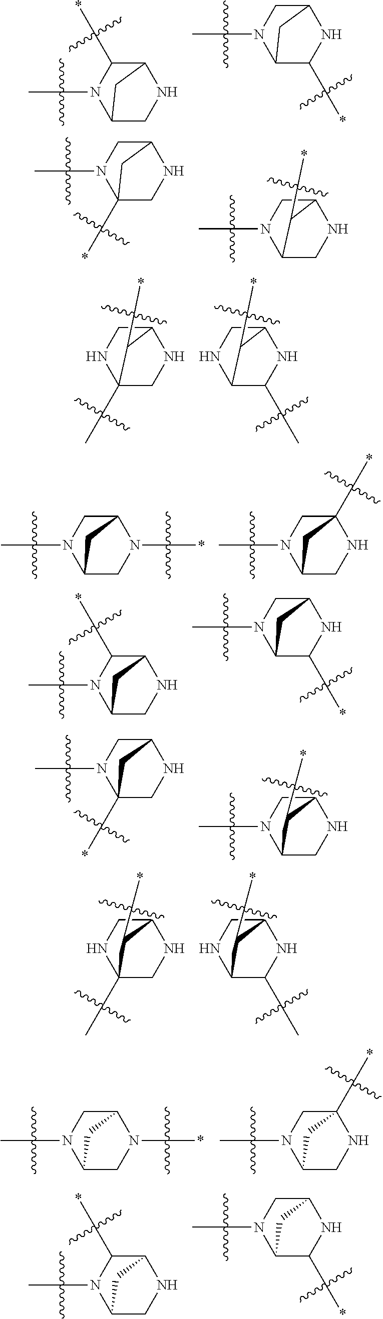 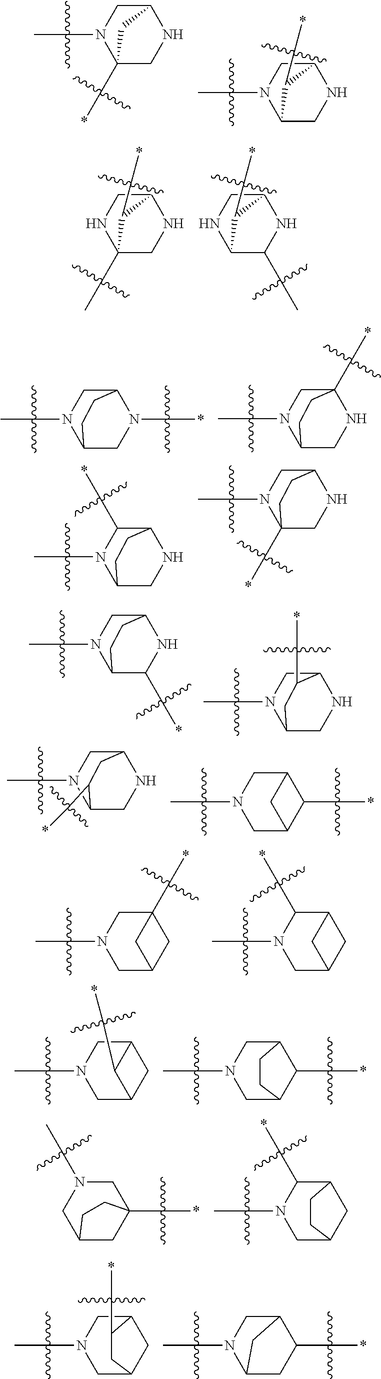 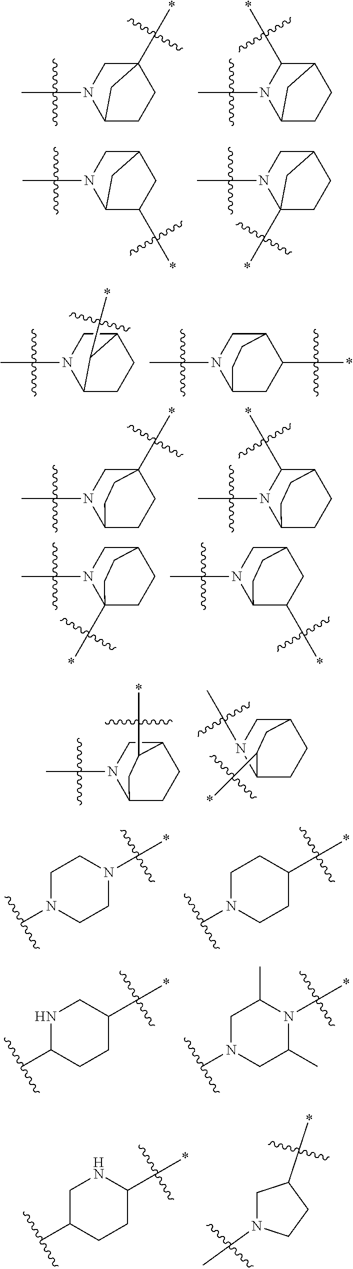 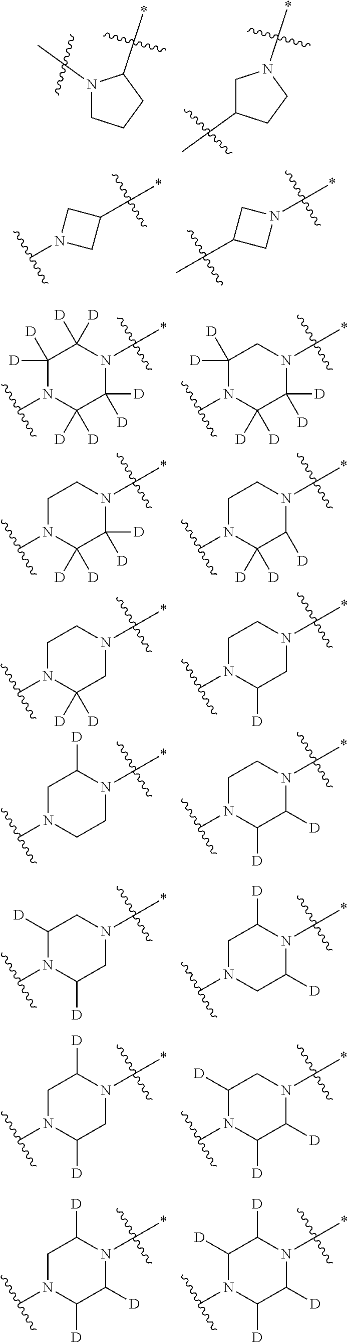 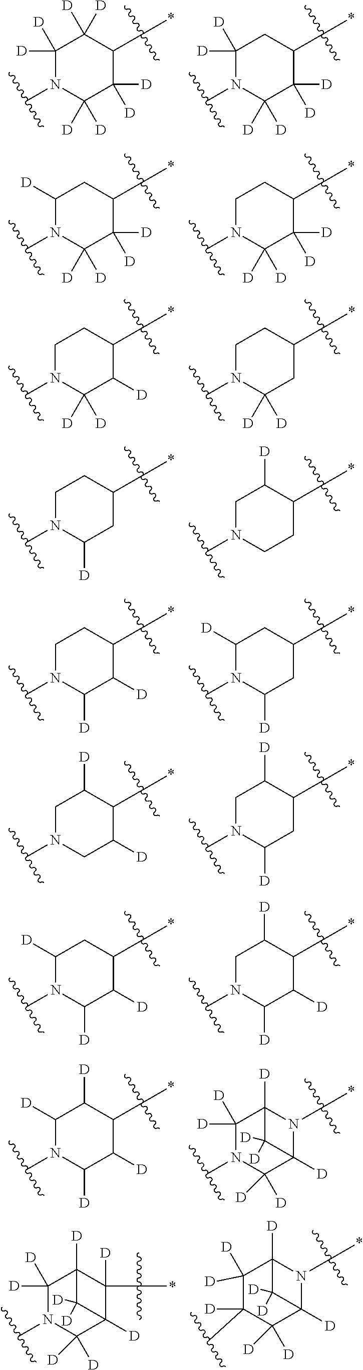 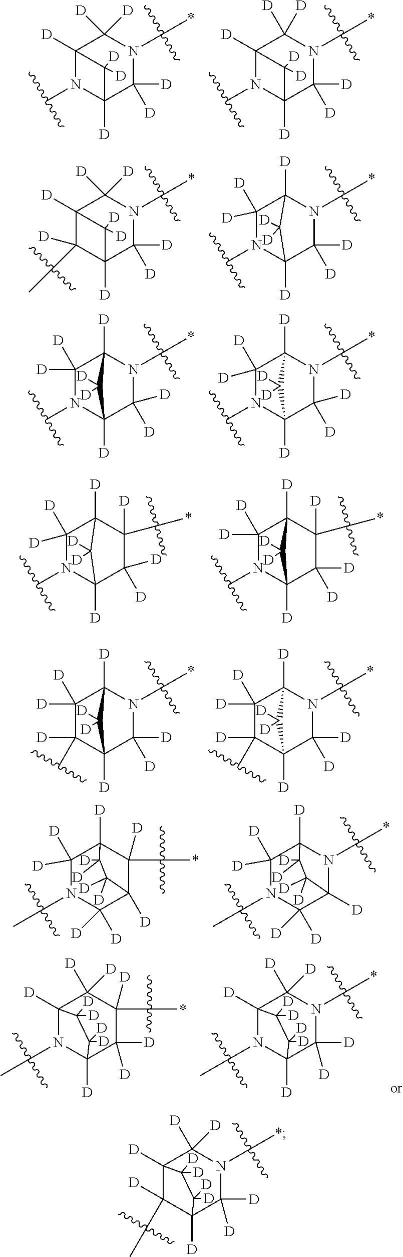
wherein symbol * indicates the point of attachment to U₁.

14-15. (canceled)

16. The compound of Formula (II) or salts, enantiomers, stereoisomers, solvates, isotopically enriched analogs, prodrugs, or polymorphs thereof as claimed in claim 9, wherein LIN represents the structure of the following formula:
#-(C(R^{a1})(R^{a2}))ₙ₁—(O(C(R^{a3})(R^{a4}))ₙ₂)ₘ₁—;
#-(C(R^{a1})(R^{a2}))ₙ₁—(O(C(R^{a3})(R^{a4}))ₙ₂)ₘ₁—(O(C(R^{a5})(R^{a6}))ₙ₃)ₘ₂—;
#-(C(R^{a1})(R^{a2}))ₙ₁—(O(C(R^{a3})(R^{a4}))ₙ₂)ₘ₁—(O(C(R^{a5})(R^{a6}))ₙ₃)ₘ₂—(O(C(R^{a7})(R^{a8}))ₙ₄)ₘ₃—;
#-(C(R^{a1})(R^{a2}))ₙ₁—(N(R_{g})(C(R^{a3})(R^{a4}))ₙ₂)ₘ₁—;
#-(C(R^{a1})(R^{a2}))ₙ₁—(N(R_{g})(C(R^{a3})(R^{a4}))ₙ₂)ₘ₁—(N(R_{g})(C(R^{a5})(R^{a6}))ₙ₃)ₘ₂—;
#-(C(R^{a1})(R^{a2}))ₙ₁—(N(R_{g})(C(R^{a3})(R^{a4}))ₙ₂)ₘ₁—(N(R_{g})(C(R^{a5})(R^{a6}))ₙ₃)ₘ₂—(N(R_{g})(C(R^{a7})(R^{a8}))ₙ₄)ₘ₃—;
#-(C(R^{a1})(R^{a2}))ₙ₁—(C(O)N(R_{g})—(C(R^{a3})(R^{a4}))ₙ₂)ₘ₁—;
#-(C(R^{a1})(R^{a2}))ₙ₁—(C(O)N(R_{g})—(C(R^{a3})(R^{a4}))ₙ₂)ₘ₁—(C(O)N(R_{g})—(C(R^{a5})(R^{a6}))ₙ₃)ₘ₂—;
#-(C(R^{a1})(R^{a2}))ₙ₁—(C(O)N(R_{g})—(C(R^{a3})(R^{a4}))ₙ₂)ₘ₁—(C(O)N(R_{g})—(C(R^{a5})(R^{a6}))ₙ₃)ₘ₂—(C(O)N(R_{g})—(C(R^{a7})(R^{a8}))ₙ₄)ₘ₃—;
#-(C(R^{a1})(R^{a2}))ₙ₁—(C(O)N(R_{g})—(C(R^{a3})(R^{a4}))ₙ₂)ₘ₁—(O—(C(R^{a5})(R^{a6}))ₙ₃)ₘ₂—;
#-(C(R^{a1})(R^{a2}))ₙ₁—C(O)N(R_{g})—(C(R^{a3})(R^{a4}))ₙ₂—(O(C(R^{a5})(R^{a6}))ₙ₃)ₘ₁—;
#-(C(R^{a1})(R^{a2}))ₙ₁—(N(R_{g})C(O)—(C(R^{a3})(R^{a4}))ₙ₂)ₘ₁—;
#-(C(R^{a1})(R^{a2}))ₙ₁—(N(R_{g})C(O)—(C(R^{a3})(R^{a4}))ₙ₂)ₘ₁—(O—(C(R^{a5})(R^{a6}))ₙ₃)ₘ₂—;
#-(C(R^{a1})(R^{a2}))ₙ₁—(N(R_{g})C(O)—(C(R^{a3})(R^{a4}))ₙ₂)ₘ₁—(O—(C(R^{a5})(R^{a6}))ₙ₃)ₘ₂—(O—(C(R_{g})(R^{a8}))ₙ₄)ₘ₃—;
#-(C(R^{a1})(R^{a2}))ₙ₁—N(R_{g})C(O)—(C(R^{a3})(R^{a4}))ₙ₂—(O(C(R^{a5})(R^{a6}))ₙ₃)ₘ₁—;
#-(C(R^{a1})(R^{a2}))ₙ₁—N(R_{g})C(O)N(R_{g})—(C(R^{a3})(R^{a4}))ₙ₂—;
#-(C(R^{a1})(R^{a2}))ₙ₁—C(O)—(C(R^{a3})(R^{a4}))ₙ₂—;
#-(C(R^{a1})(R^{a2}))ₙ₁-(arylene-(C(R^{a3})(R^{a4}))ₙ₂)ₘ₁—;
#-(C(R^{a1})(R^{a2}))ₙ₁-(arylene-(C(R^{a3})(R^{a4}))ₙ₂)ₘ₁-arylene-(C(R^{a5})(R^{a6}))ₙ₃—;
#-(C(R^{a1})(R^{a2}))ₙ₁-(heterocyclylene-(C(R^{a3})(R^{a4}))ₙ₂)ₘ₁—;
#-(C(R^{a1})(R^{a2}))ₙ₁-(heterocyclylene-(C(R^{a3})(R^{a4}))ₙ₂)ₘ₁-(heterocyclylene-(C(R^{a5})(R^{a6}))ₙ₃)ₘ₂—;
#-(C(R^{a1})(R^{a2}))ₙ₁-(heteroarylene-(C(R^{a3})(R^{a4}))ₙ₂)ₘ₁—;
#-(C(R^{a1})(R^{a2}))ₙ₁-(heteroarylene-(C(R^{a3})(R^{a4}))ₙ₂)ₘ₁-(heteroarylene-(C(R^{a5})(R^{a6}))ₙ₃)ₘ₂—;
#-(C(R^{a1})(R^{a2}))ₙ₁-(cycloalkylene-(C(R^{a3})(R^{a4}))ₙ₂)ₘ₁—; or
#-(C(R^{a1})(R^{a2}))ₙ₁-(cycloalkylene-(C(R^{a3})(R^{a4}))ₙ₂)ₘ₁-(cycloalkylene-(C(R^{a5})(R^{a6}))ₙ₃)ₘ₂—;
wherein each R_{g} independently represent H or C₁₋₆ alkyl;
the cycloalkylene, arylene, heterocyclylene, and heteroarylene are each independently optionally substituted with a substituent selected from the group consisting of deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano, or any combination thereof;
R^{a1}, R^{a2}, R^{a3}, R^{a4}, R^{a5}, R^{a6}, R^{a7} and R^{a8} each independently represent H, deuterium, hydroxy, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH—, or any combination thereof;
symbol # indicates the point of attachment to U₂; and
n1, n2, n3, n4, m1, m2, and m3 each independently represent an integer of 1, 2, 3, 4, 5, 6, 7, 8, 9, 10, 11, 12, 13, 14, or 15.

17. The compound of Formula (II) or salts, enantiomers, stereoisomers, solvates, isotopically enriched analogs, prodrugs, or polymorphs thereof as claimed in claim 9, wherein LIN represents the following groups:
#-CH₂—, #-(CH₂)₂—, #-(CH₂)₃—, #-(CH₂)₄—, #-(CH₂)₅—, #-(CH₂)₆—, #-(CH₂)₇—, #- (CH₂)₈—, #-(CH₂)₉—, #-(CH₂)₁₀—, #-(CH₂)₁₁—, #-(CH₂)₁₂—, #-(CH₂)₁₃—, #-(CH₂)₁₄—, #-(CH₂)₁₅—, #-(CH₂)₁₆—, #-(CH₂)₁₇—, #-(CH₂)₁₈—, #-(CH₂)₁₉—, #-(CH₂)₂₀—, #-(CH₂)₂₁—, #-(CH₂)₂₂—, #-(CH₂)₂₅—, or #-(CH₂)₃₀—; #-CH₂—O—CH₂—, #-CH₂—O—(CH₂)₂—, #-(CH₂)₁—O—(CH₂)₃—, #-(CH₂)₁—O—(CH₂)₄—, #-(CH₂)₁—O—(CH₂)₅—, #-(CH₂)₁—O—(CH₂)₆—, #-(CH₂)₁—O—(CH₂)₇—, #-(CH₂)₁—O—(CH₂)₈—, #-(CH₂)₁—O—(CH₂)₉—, #-(CH₂)₁—O—(CH₂)₁₀—, #-(CH₂)₂—O—(CH₂)₁—, #-(CH₂)₂—O—(CH₂)₂—, #-(CH₂)₂—O—(CH₂)₃—, #-(CH₂)₂—O—(CH₂)₄—, #-(CH₂)₂—O—(CH₂)₅—, #-(CH₂)₂—O—(CH₂)₆—, #-(CH₂)₂—O—(CH₂)₇—, #-(CH₂)₂—O—(CH₂)₈—, #-(CH₂)₂—O—(CH₂)₉—, #-(CH₂)₂—O—(CH₂)₁₀—, #-(CH₂)₂—O—(CH₂)₁₁—, #-(CH₂)₂—O—(CH₂)₁₂—, #-(CH₂)₃—O—(CH₂)₁—, #-(CH₂)₃—O—(CH₂)₂—, #-(CH₂)₃—O—(CH₂)₃—, #-(CH₂)₃—O—(CH₂)₄—, #-(CH₂)₃—O—(CH₂)₅—, #-(CH₂)₃—O—(CH₂)₆—, #-(CH₂)₃—O—(CH₂)₇—, #-(CH₂)₄—O—(CH₂)₁—, #-(CH₂)₄—O—(CH₂)₂—, #-(CH₂)₄—O—(CH₂)₃—, #-(CH₂)₄—O—(CH₂)₄—, #-(CH₂)₄—O—(CH₂)₅—, #-(CH₂)₄—O—(CH₂)₆—, #-(CH₂)₅—O—(CH₂)₁—, #-(CH₂)₅—O—(CH₂)₂—, #-(CH₂)₅—O—(CH₂)₃—, #-(CH₂)₅—O—(CH₂)₄—, #-(CH₂)₅—O—(CH₂)₅—, #-(CH₂)₆—O—(CH₂)₁—, #-(CH₂)₆—O—(CH₂)₂—, #-(CH₂)₆—O—(CH₂)₃—, #-(CH₂)₆—O—(CH₂)₄—, #-(CH₂)₇—O—(CH₂)₁—, #-(CH₂)₇—O—(CH₂)₂—, #-(CH₂)₇—O—(CH₂)₃—, #-(CH₂)₈—O—(CH₂)₁—, #-(CH₂)₈—O—(CH₂)₂—, #-CH(CH₃)—O—(CH₂)₁—, #-CH(CH₃)—O—(CH₂)₂—, #-CH(CH₃)—O—(CH₂)₃—, #-CH(CH₃)—O—(CH₂)₄—, #-CH(CH₃)—O—(CH₂)₅—, #-CH(CH₃)—O—(CH₂)₆—, #-CH(CH₃)—O—(CH₂)₇—, #-CH(CH₃)—O—(CH₂)₈—, #-CH(CH₃)—O—(CH₂)₉—, #-CH(CH₃)—O—(CH₂)₁₀—, #-CH₂—(O(CH₂)₂)₂—, #-CH₂—(O(CH₂)₂)₃—, #-CH₂—(O(CH₂)₂)₄—, #-CH₂—(O(CH₂)₂)₅—, #-CH₂—(O(CH₂)₂)₆—, #-CH₂—(O(CH₂)₂)₇—, #-CH₂—(O(CH₂)₂)₈—, #-CH₂—(O(CH₂)₂)₉—, #-CH₂—(O(CH₂)₂)₁₀—, #-CH₂—(O(CH₂)₂)₁—OCH₂—, #-CH₂—(O(CH₂)₂)₂—OCH₂—, #-CH₂—(O(CH₂)₂)₃—OCH₂—, #-CH₂—(O(CH₂)₂)₄—OCH₂—, #-CH₂—(O(CH₂)₂)₅—OCH₂—, #-CH₂—(O(CH₂)₂)₆—OCH₂—, #-CH₂—(O(CH₂)₂)₇—OCH₂—, #-CH₂—(O(CH₂)₂)₈—OCH₂—, #-CH₂—(O(CH₂)₂)₉—OCH₂—, #-CH₂—(O(CH₂)₂)₁₀—OCH₂—, #-(CH₂)₂—(O(CH₂)₂)₂—, #-(CH₂)₂—(O(CH₂)₂)₃—, #-(CH₂)₂—(O(CH₂)₂)₄—, #-(CH₂)₂—(O(CH₂)₂)₅—, #-(CH₂)₂—(O(CH₂)₂)₆—, #-(CH₂)₂—(O(CH₂)₂)₇—, #-(CH₂)₂—(O(CH₂)₂)₈—, #-(CH₂)₂—(O(CH₂)₂)₉—, #-(CH₂)₂—(O(CH₂)₂)₁₀—, #-(CH₂)₃—(O(CH₂)₂)₂—, #-(CH₂)₃—(O(CH₂)₂)₃—, #-(CH₂)₃—(O(CH₂)₂)₄—, #-(CH₂)₃—(O(CH₂)₂)₅—, #-(CH₂)₃—(O(CH₂)₂)₆—, #-(CH₂)₃—(O(CH₂)₂)₇—, #-(CH₂)₃—(O(CH₂)₂)₈—, #-(CH₂)₃—(O(CH₂)₂)₉—, #-(CH₂)₃—(O(CH₂)₂)₁₀—, #-(CH₂)₄—(O(CH₂)₂)₂—, #-(CH₂)₄—(O(CH₂)₂)₃—, #-(CH₂)₄—(O(CH₂)₂)₄—, #-(CH₂)₄—(O(CH₂)₂)₅—, #-(CH₂)₄—(O(CH₂)₂)₆—, #-(CH₂)₄—(O(CH₂)₂)₇—, #-(CH₂)₄—(O(CH₂)₂)₈—, #-(CH₂)₄—(O(CH₂)₂)₉—, #-(CH₂)₄—(O(CH₂)₂)₁₀—, #-CH₂—(O(CH₂)₃)₂—, #-CH₂—(O(CH₂)₃)₃—, #-CH₂—(O(CH₂)₃)₄—, #-CH₂—(O(CH₂)₃)₅—, #-CH₂—(O(CH₂)₃)₆—, #-CH₂—(O(CH₂)₃)₇—, #-CH₂—(O(CH₂)₃)₈—, #-CH₂—(O(CH₂)₃)₉—, #- CH₂—(O(CH₂)₃)₁₀—, #-(CH₂)₂—(O(CH₂)₃)₂—, #-(CH₂)₂—(O(CH₂)₃)₃—, #-(CH₂)₂—(O(CH₂)₃)₄—, #-(CH₂)₂—(O(CH₂)₃)₅—, #-(CH₂)₂—(O(CH₂)₃)₆—, #-(CH₂)₂—(O(CH₂)₃)₇—, #-(CH₂)₂—(O(CH₂)₃)₈—, #-(CH₂)₂—(O(CH₂)₃)₉—, #-(CH₂)₂—(O(CH₂)₃)₁₀—, #-(CH₂)₃—(O(CH₂)₃)₂—, #-(CH₂)₃—(O(CH₂)₃)₃—, #-(CH₂)₃—(O(CH₂)₃)₄—, #-(CH₂)₃—(O(CH₂)₃)₅—, #-(CH₂)₃—(O(CH₂)₃)₆—, #-(CH₂)₃—(O(CH₂)₃)₇—, #-(CH₂)₃—(O(CH₂)₃)₈—, #-(CH₂)₃—(O(CH₂)₃)₉—, #-(CH₂)₃—(O(CH₂)₃)₁₀—, #-CH₂—O—(CH₂)₂—O—(CH₂)₃—, #-CH₂—(O(CH₂)₂)₂—(O(CH₂)₃)₂—, #-CH₂—(O(CH₂)₂)₃—(O(CH₂)₃)₃—, #-CH₂—(O(CH₂)₂)₄—(O(CH₂)₃)₄—, #-CH₂—(O(CH₂)₂)₅—(O(CH₂)₃)₅—, #-CH₂—(O(CH₂)₂)₆—(O(CH₂)₃)₆—, #-(CH₂)₂—O—(CH₂)₂—O—(CH₂)₃—, #-(CH₂)₂—(O(CH₂)₂)₂—(O(CH₂)₃)₂—, #-(CH₂)₂—(O(CH₂)₂)₃—(O(CH₂)₃)₃—, #-(CH₂)₂—(O(CH₂)₂)₄—(O(CH₂)₃)₄—, #-(CH₂)₂—(O(CH₂)₂)₅—(O(CH₂)₃)₅—, #-(CH₂)₂—(O(CH₂)₂)₆—(O(CH₂)₃)₆—, #-(CH₂)₃—O—(CH₂)₂—O—(CH₂)₃—, #-(CH₂)₃—(O(CH₂)₂)₂—(O(CH₂)₃)₂—, #-(CH₂)₃—(O(CH₂)₂)₃—(O(CH₂)₃)₃—, #-(CH₂)₃—(O(CH₂)₂)₄—(O(CH₂)₃)₄—, #-(CH₂)₃—(O(CH₂)₂)₅—(O(CH₂)₃)₅—, #-(CH₂)₃—(O(CH₂)₂)₆—(O(CH₂)₃)₆—, #-CH₂—O—(CH₂)₃—O—(CH₂)₂—, #-CH₂—(O(CH₂)₃)₂—(O(CH₂)₂)₂—, #-CH₂—(O(CH₂)₃)₃—(O(CH₂)₂)₃—, #-CH₂—(O(CH₂)₃)₄—(O(CH₂)₂)₄—, #-CH₂—(O(CH₂)₃)₅—(O(CH₂)₂)₅—, #-CH₂—(O(CH₂)₃)₆—(O(CH₂)₂)₆—, #-(CH₂)₂—O—(CH₂)₃—O—(CH₂)₂—, #-(CH₂)₂—(O(CH₂)₃)₂—(O(CH₂)₂)₂—, #-(CH₂)₂—(O(CH₂)₃)₃—(O(CH₂)₂)₃—, #-(CH₂)₂—(O(CH₂)₃)₄—(O(CH₂)₂)₄—, #-(CH₂)₂—(O(CH₂)₃)₅—(O(CH₂)₂)₅—, #-(CH₂)₂—(O(CH₂)₃)₆—(O(CH₂)₂)₆—, #-(CH₂)₃—O—(CH₂)₃—O—(CH₂)₂—, #-(CH₂)₃—(O(CH₂)₃)₂—(O(CH₂)₂)₂—, #-(CH₂)₃—(O(CH₂)₃)₃—(O(CH₂)₂)₃—, #-(CH₂)₃—(O(CH₂)₃)₄—(O(CH₂)₂)₄—, #-(CH₂)₃—(O(CH₂)₃)₅—(O(CH₂)₂)₅—, #-(CH₂)₃—(O(CH₂)₃)₆—(O(CH₂)₂)₆—, #-CH₂—O—(CH₂)₂O—CH₂—, #-(CH₂)₂—O—(CH₂)₂O—CH₂—, #-(CH₂)₂—(O(CH₂)₂)₂—O—(CH₂)₃—, #-(CH₂)₂—(O(CH₂)₂)₃—O—(CH₂)₃—, #-(CH₂)₂—(O(CH₂)₂)₄—O—(CH₂)₃—, #-(CH₂)₅—(O(CH₂)₂)₂—O—(CH₂)₅—, #-(CH₂)₅—(O(CH₂)₂)₂—O—(CH₂)₆—, #-(CH₂)₁—N(R_{g})—(CH₂)₁—, #-(CH₂)₁—N(R_{g})—(CH₂)₂—, #-(CH₂)₁—N(R_{g})—(CH₂)₃—, #-(CH₂)₁—N(R_{g})—(CH₂)₄—, #-(CH₂)₁—N(R_{g})—(CH₂)₅—, #-(CH₂)₁—N(R_{g})—(CH₂)₆—, #-(CH₂)₁—N(R_{g})—(CH₂)₇—, #-(CH₂)₁—N(R_{g})—(CH₂)₈—, #-(CH₂)₁—N(R_{g})—(CH₂)₉—, #-(CH₂)₁—N(R_{g})—(CH₂)₁₀—, #-(CH₂)₂—N(R_{g})—(CH₂)₁—, #-(CH₂)₂—N(R_{g})—(CH₂)₂—, #-(CH₂)₂—N(R_{g})—(CH₂)₃—, #-(CH₂)₂—N(R_{g})—(CH₂)₄—, #-(CH₂)₂—N(R_{g})—(CH₂)₅—, #-(CH₂)₂—N(R_{g})—(CH₂)₆—, #-(CH₂)₂—N(R_{g})—(CH₂)₇—, #-(CH₂)₂—N(R_{g})—(CH₂)₈—, #-(CH₂)₂—N(R_{g})—(CH₂)₉—, #-(CH₂)₂—N(R_{g})—(CH₂)₁₀—, #-(CH₂)₂—N(R_{g})—(CH₂)₁₁—, #-(CH₂)₂—N(R_{g})—(CH₂)₁₂—, #-(CH₂)₃—N(R_{g})—(CH₂)₁—, #-(CH₂)₃—N(R_{g})—(CH₂)₂—, #-(CH₂)₃—N(R_{g})—(CH₂)₃—, #-(CH₂)₄—N(R_{g})—(CH₂)₁—, #-(CH₂)₄—N(R_{g})—(CH₂)₂—, #-(CH₂)₄—N(R_{g})—(CH₂)₃—, #-(CH₂)₄—N(R_{g})—(CH₂)₄—, #-(CH₂)₅—N(R_{g})—(CH₂)₁—, #-(CH₂)₅—N(R_{g})—(CH₂)₂—, #-(CH₂)₅—N(R_{g})—(CH₂)₃—, #-(CH₂)₅—N(R_{g})—(CH₂)₄—, #-(CH₂)₅—N(R_{g})—(CH₂)₅—, #-(CH₂)₆—N(R_{g})—(CH₂)₁—, #-(CH₂)₆—N(R_{g})—(CH₂)₂—, #-(CH₂)₆—N(R_{g})—(CH₂)₃—, #-(CH₂)₇—N(R_{g})—(CH₂)₁—, #-(CH₂)₇—N(R_{g})—(CH₂)₂—, #-(CH₂)₇—N(R_{g})—(CH₂)₃—, #-(CH₂)₈—N(R_{g})—(CH₂)₁—, #-(CH₂)₈—N(R_{g})—(CH₂)₂—, #-(CH₂)₈—N(R_{g})—(CH₂)₃—, #-CH(CH₃)—N(R_{g})—(CH₂)₁—, #-CH(CH₃)—N(R_{g})—(CH₂)₂—, #-CH(CH₃)—N(R_{g})—(CH₂)₃—, #-CH(CH₃)—N(R_{g})—(CH₂)₄—, #-CH(CH₃)—N(R_{g})—(CH₂)₅—, #-CH(CH₃)—N(R_{g})—(CH₂)₆—, #-CH(CH₃)—N(R_{g})—(CH₂)₇—, #-CH(CH₃)—N(R_{g})—(CH₂)₈—, #-CH(CH₃)—N(R_{g})—(CH₂)₉—, #-CH(CH₃)—N(R_{g})—(CH₂)₁₀—, #-CH₂C(O)NHCH₂—, #-(CH₂)₂C(O)NH(CH₂)₂—, #-(CH₂)₂C(O)NH(CH₂)₃—, #-(CH₂)₂C(O)NH(CH₂)₄—, #-(CH₂)₂C(O)NH(CH₂)₅—, #-(CH₂)₃C(O)NH(CH₂)₃—, #-(CH₂)₃C(O)NH(CH₂)₄—, #-(CH₂)₄C(O)NH(CH₂)₄—, #-(CH₂)₅C(O)NH(CH₂)₅—, #-(CH₂)₆C(O)NH(CH₂)₇—, #-(CH₂)₆C(O)NH(CH₂)₆—, #-(CH₂)₇C(O)NH(CH₂)₇—, #-(CH₂)₈C(O)NH(CH₂)₈, U—(CH₂)₉C(O)NH(CH₂)₉—, #-(CH₂)₁₀C(O)NH(CH₂)₁₀—, #-(CH₂)₂C(O)NH(CH₂)₂—O—(CH₂)₂—, #-CH₂NHC(O)CH₂—, #-(CH₂)₂NHC(O)(CH₂)₂—, #-(CH₂)₂NHC(O)(CH₂)₃—, #-(CH₂)₂NHC(O)(CH₂)₄—, #-(CH₂)₂NHC(O)(CH₂)₅—, #-(CH₂)₃NHC(O)(CH₂)₃—, #-(CH₂)₃NHC(O)(CH₂)₄—, #-(CH₂)₄NHC(O)(CH₂)₄—, #-(CH₂)₅NHC(O)(CH₂)₅—, #-(CH₂)₆NHC(O)(CH₂)₇—, #-(CH₂)₆NHC(O)(CH₂)₆—, #-(CH₂)₇NHC(O)(CH₂)₇—, #-(CH₂)₈NHC(O)(CH₂)₈, #-(CH₂)₉NHC(O)(CH₂)₉—, #-(CH₂)₁₀NHC(O)(CH₂)₁₀—, #-(CH₂)₄NHC(O)(CH₂)₈—, #-(CH₂)₂NHC(O)(CH₂)₂—O—(CH₂)₂—, #-(CH₂)₄NHC(O)CH₂—, #-CH₂-piperidinylene-CH₂—, #-CH₂-piperidinylene-(CH₂)₂—, #-CH₂-piperidinylene-(CH₂)₃—, #-CH₂-piperidinylene-(CH₂)₄—, #-CH₂-piperidinylene-(CH₂)₅—, #-CH₂-piperidinylene-(CH₂)₆—, #-CH₂-piperidinylene-(CH₂)₇—, #-CH₂-piperidinylene-(CH₂)₈—, #-(CH₂)₂-piperidinylene-(CH₂)₁—, #-(CH₂)₂-piperidinylene-(CH₂)₂—, #-(CH₂)₂-piperidinylene-(CH₂)₃—, #-(CH₂)₂-piperidinylene-(CH₂)₄—, #-(CH₂)₂-piperidinylene-(CH₂)₅—, #-(CH₂)₂-piperidinylene-(CH₂)₆—, #-(CH₂)₂-piperidinylene-(CH₂)₇—, #-(CH₂)₂-piperidinylene-(CH₂)₈—, #-(CH₂)₃-piperidinylene-CH₂—, #-(CH₂)₃-piperidinylene-(CH₂)₂—, #-(CH₂)₃-piperidinylene-(CH₂)₃—, #-(CH₂)₃-piperidinylene-(CH₂)₄—, #-(CH₂)₃-piperidinylene-(CH₂)₅—, #-(CH₂)₃-piperidinylene-(CH₂)₆—, #-(CH₂)₃-piperidinylene-(CH₂)₇—, #-(CH₂)₃-piperidinylene-(CH₂)₈—, #-(CH₂)₄-piperidinylene-CH₂—, #-(CH₂)₄-piperidinylene-(CH₂)₂—, #-(CH₂)₄-piperidinylene-(CH₂)₃—, #-(CH₂)₄-piperidinylene-(CH₂)₄—, #-(CH₂)₄-piperidinylene-(CH₂)₅—, #-(CH₂)₄-piperidinylene-(CH₂)₆—, #-(CH₂)₄-piperidinylene-(CH₂)₇—, #-(CH₂)₄-piperidinylene-(CH₂)₈—, #-(CH₂)₅-piperidinylene-(CH₂)₁—, #-(CH₂)₅-piperidinylene-(CH₂)₂—, #-(CH₂)₅-piperidinylene-(CH₂)₃—, #-(CH₂)₅-piperidinylene-(CH₂)₄—, #-(CH₂)₅-piperidinylene-(CH₂)₅—, #-(CH₂)₅-piperidinylene-(CH₂)₆—, #-(CH₂)₅-piperidinylene-(CH₂)₇—, #-(CH₂)₅-piperidinylene-(CH₂)₈—, #-(CH₂)₆-piperidinylene-(CH₂)₁—, #-(CH₂)₆-piperidinylene-(CH₂)₂—, #-(CH₂)₆-piperidinylene-(CH₂)₃—, #-(CH₂)₆-piperidinylene-(CH₂)₄—, #-(CH₂)₆-piperidinylene-(CH₂)₅—, #-(CH₂)₆-piperidinylene-(CH₂)₆—, #-(CH₂)₆-piperidinylene-(CH₂)₇—, #-(CH₂)₆-piperidinylene-(CH₂)₈—, #-(CH₂)₇-piperidinylene-(CH₂)₁—, #-(CH₂)₇-piperidinylene-(CH₂)₂—, #-(CH₂)₇-piperidinylene-(CH₂)₃—, #-(CH₂)₇-piperidinylene-(CH₂)₄—, #-(CH₂)₇-piperidinylene-(CH₂)₈—, #-(CH₂)₈-piperidinylene-CH₂—, #-(CH₂)₈-piperidinylene-(CH₂)₂—, #-(CH₂)₈-piperidinylene-(CH₂)₃—, #-(CH₂)₈-piperidinylene-(CH₂)₄—, #-(CH₂)₈-piperidinylene-(CH₂)₅—, #-(CH₂)₈-piperidinylene-(CH₂)₆—, #-(CH₂)₈-piperidinylene-(CH₂)₇—, #-(CH₂)₈-piperidinylene-(CH₂)₈—, #-CH₂—N(R_{g})—CH₂-piperidinylene-CH₂—, #-CH₂—N(R_{g})—CH₂-piperidinylene-(CH₂)₂—, #-CH₂—N(R_{g})—CH₂-piperidinylene-(CH₂)₃—, #-CH₂—N(R_{g})—CH₂-piperidinylene-(CH₂)₄—, #-CH₂—N(R_{g})—CH₂-piperidinylene-(CH₂)₅—, #-CH₂—N(R_{g})—CH₂-piperidinylene-(CH₂)₆—, #-CH₂—N(R_{g})—CH₂-piperidinylene-(CH₂)₇—, #-CH₂—N(R_{g})—CH₂-piperidinylene-(CH₂)₈—, #-CH₂—N(R_{g})—(CH₂)₂-piperidinylene-CH₂—, #-CH₂—N(R_{g})—(CH₂)₂-piperidinylene-(CH₂)₂—, #-CH₂—N(R_{g})—(CH₂)₂-piperidinylene-(CH₂)₃—, #-CH₂—N(R_{g})—(CH₂)₂-piperidinylene-(CH₂)₄—, #-CH₂—N(R_{g})—(CH₂)₂-piperidinylene-(CH₂)₅—, #-CH₂—N(R_{g})—(CH₂)₂-piperidinylene-(CH₂)₆—, #-CH₂—N(R_{g})—(CH₂)₂-piperidinylene-(CH₂)₇—, #-CH₂—N(R_{g})—(CH₂)₂-piperidinylene-(CH₂)₈—, #-(CH₂)₂—N(R_{g})—CH₂-piperidinylene-CH₂—, #-(CH₂)₂—N(R_{g})—CH₂-piperidinylene-(CH₂)₂—, #-(CH₂)₂—N(R_{g})—CH₂-piperidinylene-(CH₂)₃—, #-(CH₂)₂—N(R_{g})—CH₂-piperidinylene-(CH₂)₄—, #-(CH₂)₂—N(R_{g})—CH₂-piperidinylene-(CH₂)₅—, #-(CH₂)₂—N(R_{g})—CH₂-piperidinylene-(CH₂)₆—, #-(CH₂)₂—N(R_{g})—CH₂-piperidinylene-(CH₂)₇—, #-(CH₂)₂—N(R_{g})—CH₂-piperidinylene-(CH₂)₈—, #-(CH₂)₃—N(R_{g})—CH₂-piperidinylene-CH₂—, #-(CH₂)₃—N(R_{g})—CH₂-piperidinylene-(CH₂)₂—, #-(CH₂)₃—N(R_{g})—CH₂-piperidinylene-(CH₂)₃—, #-(CH₂)₃—N(R_{g})—CH₂-piperidinylene-(CH₂)₈—, #-(CH₂)₄—N(R_{g})—CH₂-piperidinylene-CH₂—, #-(CH₂)₄—N(R_{g})—CH₂-piperidinylene-(CH₂)₂—, #-(CH₂)₄—N(R_{g})—CH₂-piperidinylene-(CH₂)₃—, #-(CH₂)₄—N(R_{g})—CH₂-piperidinylene-(CH₂)₈—, #-(CH₂)₅—N(R_{g})—CH₂-piperidinylene-(CH₂)₃—, #-(CH₂)₅—N(R_{g})—CH₂-piperidinylene-(CH₂)₈—, #-(CH₂)₆—N(R_{g})—CH₂-piperidinylene-(CH₂)₃—, #-(CH₂)₆—N(R_{g})—CH₂-piperidinylene-(CH₂)₈—, #-(CH₂)₇—N(R_{g})—CH₂-piperidinylene-(CH₂)₃—, #-(CH₂)₇—N(R_{g})—CH₂-piperidinylene-(CH₂)₈—, #-(CH₂)₈—N(R_{g})—CH₂-piperidinylene-CH₂—, #-(CH₂)₈—N(R_{g})—CH₂-piperidinylene-(CH₂)₂—, #-(CH₂)₈—N(R_{g})—CH₂-piperidinylene-(CH₂)₃—, #-(CH₂)₈—N(R_{g})—CH₂-piperidinylene-(CH₂)₄—, #-(CH₂)₈—N(R_{g})—CH₂-piperidinylene-(CH₂)₅—, #-(CH₂)₈—N(R_{g})—CH₂-piperidinylene-(CH₂)₆—, #-(CH₂)₈—N(R_{g})—CH₂-piperidinylene-(CH₂)₇—, #-(CH₂)₈—N(R_{g})—CH₂-piperidinylene-(CH₂)₈—, #-CH₂-piperazinylene-CH₂—, #-CH₂-piperazinylene-(CH₂)₂—, #-CH₂-piperazinylene-(CH₂)₃—, #-CH₂-piperazinylene-(CH₂)₄—, #-CH₂-piperazinylene-(CH₂)₅—, #-CH₂-piperazinylene-(CH₂)₆—, #-CH₂-piperazinylene-(CH₂)₇—, #-CH₂-piperazinylene-(CH₂)₈—, #-(CH₂)₂-piperazinylene-(CH₂)₁—, #-(CH₂)₂-piperazinylene-(CH₂)₂—, #-(CH₂)₂-piperazinylene-(CH₂)₃—, #-(CH₂)₂-piperazinylene-(CH₂)₄—, #-(CH₂)₂-piperazinylene-(CH₂)₅—, #-(CH₂)₂-piperazinylene-(CH₂)₆—, #-(CH₂)₂-piperazinylene-(CH₂)₇—, #-(CH₂)₂-piperazinylene-(CH₂)₈—, #-(CH₂)₃-piperazinylene-CH₂—, #-(CH₂)₃-piperazinylene-(CH₂)₂—, #-(CH₂)₃-piperazinylene-(CH₂)₃—, #-(CH₂)₃-piperazinylene-(CH₂)₄—, #-(CH₂)₃-piperazinylene-(CH₂)₅—, #-(CH₂)₃-piperazinylene-(CH₂)₆—, #-(CH₂)₃-piperazinylene-(CH₂)₇—, #-(CH₂)₃-piperazinylene-(CH₂)₈—, #-(CH₂)₄-piperazinylene-CH₂—, #-(CH₂)₄-piperazinylene-(CH₂)₂—, #-(CH₂)₄-piperazinylene-(CH₂)₃—, #-(CH₂)₄-piperazinylene-(CH₂)₄—, #-(CH₂)₄-piperazinylene-(CH₂)₅—, #-(CH₂)₄-piperazinylene-(CH₂)₆—, #-(CH₂)₄-piperazinylene-(CH₂)₇—, #-(CH₂)₄-piperazinylene-(CH₂)₈—, #-(CH₂)₅-piperazinylene-(CH₂)₁—, #-(CH₂)₅-piperazinylene-(CH₂)₂—, #-(CH₂)₅-piperazinylene-(CH₂)₃—, #-(CH₂)₅-piperazinylene-(CH₂)₄—, #-(CH₂)₅-piperazinylene-(CH₂)₅—, #-(CH₂)₅-piperazinylene-(CH₂)₆—, #-(CH₂)₅-piperazinylene-(CH₂)₇—, #-(CH₂)₅-piperazinylene-(CH₂)₈—, #-(CH₂)₆-piperazinylene-(CH₂)₁—, #-(CH₂)₆-piperazinylene-(CH₂)₂—, #-(CH₂)₆-piperazinylene-(CH₂)₃—, #-(CH₂)₆-piperazinylene-(CH₂)₄—, #-(CH₂)₆-piperazinylene-(CH₂)₅—, #-(CH₂)₆-piperazinylene-(CH₂)₆—, #-(CH₂)₆-piperazinylene-(CH₂)₇—, #-(CH₂)₆-piperazinylene-(CH₂)₈—, #-(CH₂)₇-piperazinylene-(CH₂)₁—, #-(CH₂)₇-piperazinylene-(CH₂)₂—, #-(CH₂)₇-piperazinylene-(CH₂)₃—, #-(CH₂)₇-piperazinylene-(CH₂)₄—, #-(CH₂)₇-piperazinylene-(CH₂)₈—, #-(CH₂)₈-piperazinylene-CH₂—, #-(CH₂)₈-piperazinylene-(CH₂)₂—, #-(CH₂)₈-piperazinylene-(CH₂)₃—, #-(CH₂)₈-piperazinylene-(CH₂)₄—, #-(CH₂)₈-piperazinylene-(CH₂)₅—, #-(CH₂)₈-piperazinylene-(CH₂)₆—, #-(CH₂)₈-piperazinylene-(CH₂)₇—, or #-(CH₂)₈-piperazinylene-(CH₂)₈—;
wherein the piperidinylene and the piperazinylene are each independently optionally substituted with a substituent selected from the group consisting of deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano, or any combination thereof;
each R_{g} independently represent H or C₁₋₆ alkyl;
symbol # indicates the point of attachment to U₂; and
n1, n2, n3, n4, m1, m2, and m3 each independently represent an integer of 1, 2, 3, 4, 5, 6, 7, 8, 9, 10, 11, 12, 13, 14, or 15.

18. The compound of Formula (II) or salts, enantiomers, stereoisomers, solvates, isotopically enriched analogs, prodrugs, or polymorphs thereof as claimed in claim 9, wherein Rₐ represents the following groups: 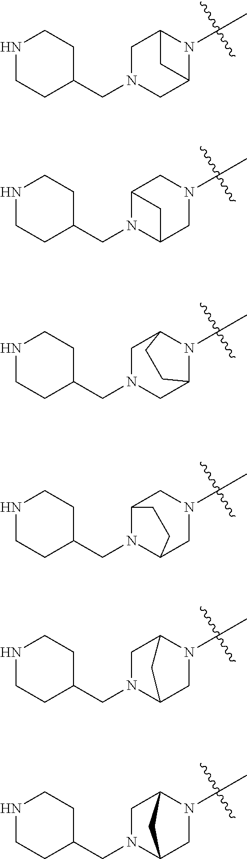 [SMILES: *N1C2CC1CN(CC1CCNCC1)C2 ; *N1CC2CC(C1)N2CC1CCNCC1 ; *N1C2CCC1CN(CC1CCNCC1)C2 ; *N1CC2CCC(C1)N2CC1CCNCC1 ; *N1CC2CC1CN2CC1CCNCC1 ; *N1C[C@H]2C[C@@H]1CN2CC1CCNCC1] 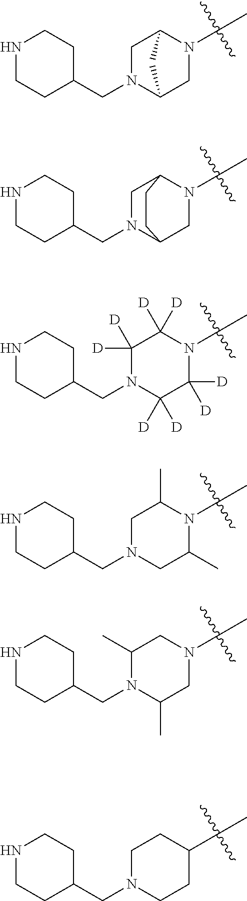 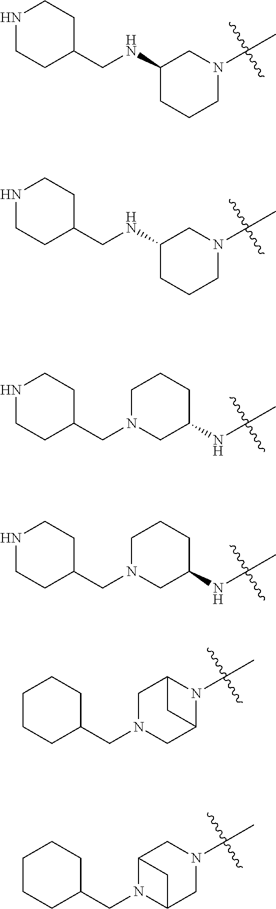 [SMILES: *N1CCC[C@@H](NCC2CCNCC2)C1 ; *N1CCC[C@H](NCC2CCNCC2)C1 ; *N[C@H]1CCCN(CC2CCNCC2)C1 ; *N[C@@H]1CCCN(CC2CCNCC2)C1 ; *N1C2CC1CN(CC1CCCCC1)C2 ; *N1CC2CC(C1)N2CC1CCCCC1] 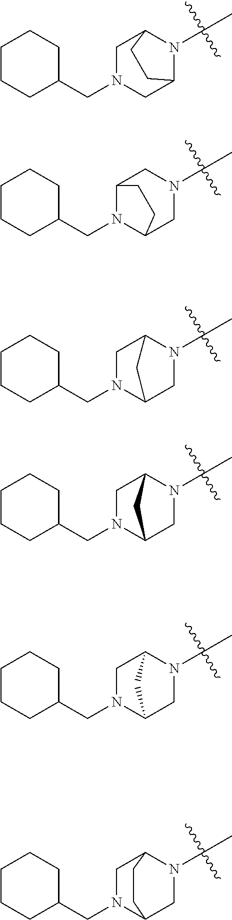 [SMILES: *N1C2CCC1CN(CC1CCCCC1)C2 ; *N1CC2CCC(C1)N2CC1CCCCC1 ; *N1CC2CC1CN2CC1CCCCC1 ; *N1C[C@H]2C[C@@H]1CN2CC1CCCCC1 ; *N1C[C@@H]2C[C@H]1CN2CC1CCCCC1 ; *N1CC2CCC1CN2CC1CCCCC1] 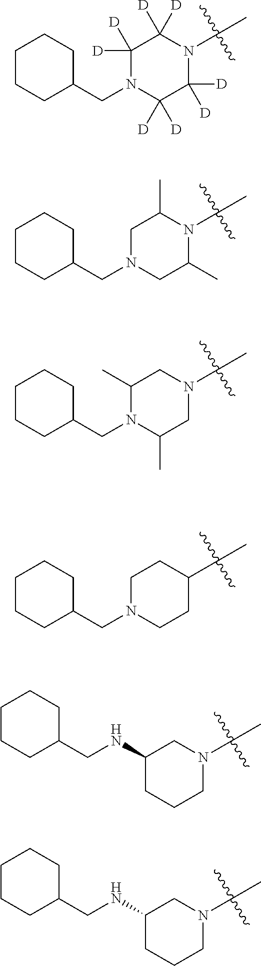 [SMILES: *N1C([2H])([2H])C([2H])([2H])N(CC2CCCCC2)C([2H])([2H])C1([2H])[2H] ; *N1C(C)CN(CC2CCCCC2)CC1C ; *N1CC(C)N(CC2CCCCC2)C(C)C1 ; *C1CCN(CC2CCCCC2)CC1 ; *N1CCC[C@@H](NCC2CCCCC2)C1 ; *N1CCC[C@H](NCC2CCCCC2)C1] 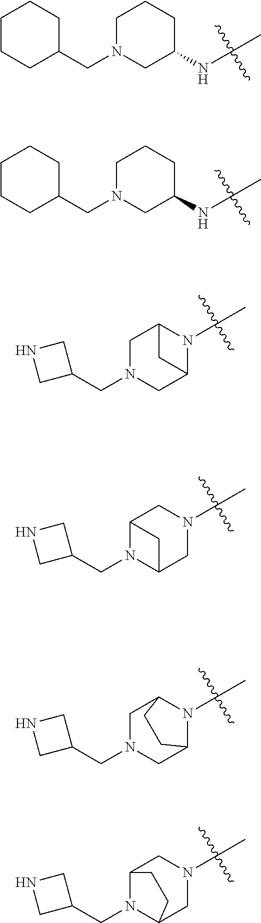 [SMILES: *N[C@H]1CCCN(CC2CCCCC2)C1 ; *N[C@@H]1CCCN(CC2CCCCC2)C1 ; *N1C2CC1CN(CC1CNC1)C2 ; *N1CC2CC(C1)N2CC1CNC1 ; *N1C2CCC1CN(CC1CNC1)C2 ; *N1CC2CCC(C1)N2CC1CNC1] 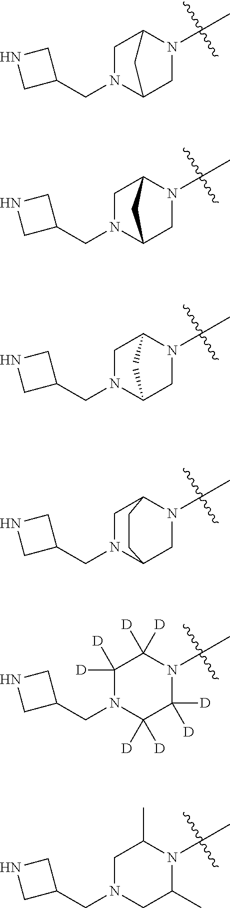 [SMILES: *N1CC2CC1CN2CC1CNC1 ; *N1C[C@H]2C[C@@H]1CN2CC1CNC1 ; *N1C[C@@H]2C[C@H]1CN2CC1CNC1 ; *N1CC2CCC1CN2CC1CNC1 ; *N1C([2H])([2H])C([2H])([2H])N(CC2CNC2)C([2H])([2H])C1([2H])[2H] ; *N1C(C)CN(CC2CNC2)CC1C] 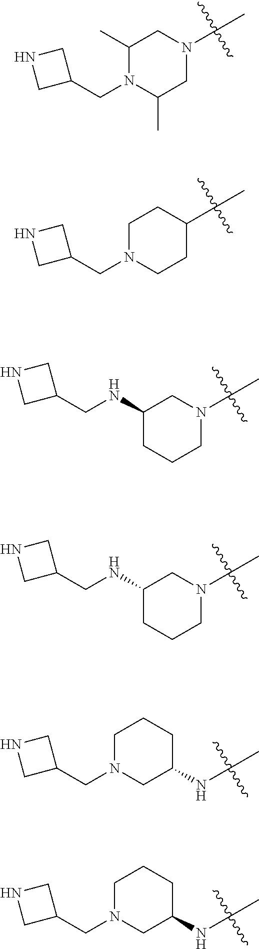 [SMILES: *N1CC(C)N(CC2CNC2)C(C)C1 ; *C1CCN(CC2CNC2)CC1 ; *N1CCC[C@@H](NCC2CNC2)C1 ; *N1CCC[C@H](NCC2CNC2)C1 ; *N[C@H]1CCCN(CC2CNC2)C1 ; *N[C@@H]1CCCN(CC2CNC2)C1] 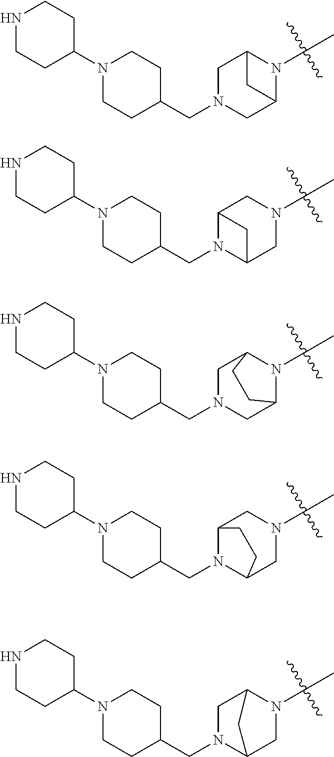 [SMILES: *N1C2CC1CN(CC1CCN(C3CCNCC3)CC1)C2 ; *N1CC2CC(C1)N2CC1CCN(C2CCNCC2)CC1 ; *N1C2CCC1CN(CC1CCN(C3CCNCC3)CC1)C2 ; *N1CC2CCC(C1)N2CC1CCN(C2CCNCC2)CC1 ; *N1CC2CC1CN2CC1CCN(C2CCNCC2)CC1] 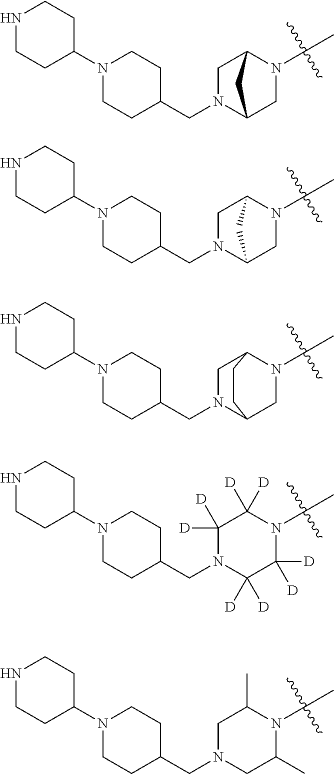 [SMILES: *N1C[C@H]2C[C@@H]1CN2CC1CCN(C2CCNCC2)CC1 ; *N1C[C@@H]2C[C@H]1CN2CC1CCN(C2CCNCC2)CC1 ; *N1CC2CCC1CN2CC1CCN(C2CCNCC2)CC1 ; *N1C([2H])([2H])C([2H])([2H])N(CC2CCN(C3CCNCC3)CC2)C([2H])([2H])C1([2H])[2H] ; *N1C(C)CN(CC2CCN(C3CCNCC3)CC2)CC1C] 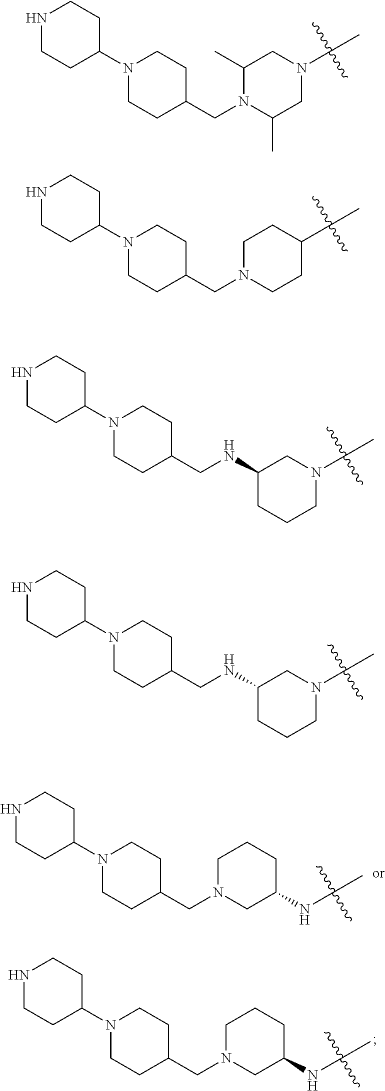 [SMILES: *N1CC(C)N(CC2CCN(C3CCNCC3)CC2)C(C)C1 ; *C1CCN(CC2CCN(C3CCNCC3)CC2)CC1 ; *N1CCC[C@@H](NCC2CCN(C3CCNCC3)CC2)C1 ; *N1CCC[C@H](NCC2CCN(C3CCNCC3)CC2)C1 ; *N[C@H]1CCCN(CC2CCN(C3CCNCC3)CC2)C1 ; *N[C@@H]1CCCN(CC2CCN(C3CCNCC3)CC2)C1]
wherein the groups are optionally partially or fully deuterated.

19. The compound of Formula (II) or salts, enantiomers, stereoisomers, solvates, isotopically enriched analogs, prodrugs, or polymorphs thereof as claimed in claim 9, which is selected from:
2-(2,6-dioxopiperidin-3-yl)-5-(3-(piperidin-4-ylmethyl)-3,6-diazabicyclo[3.1.1]heptan-6-yl)isoindoline-1,3-dione;
2-(2,6-dioxopiperidin-3-yl)-5-fluoro-6-(3-(piperidin-4-ylmethyl)-3,6-diazabicyclo[3.1.1]heptan-6-yl)isoindoline-1,3-dione;
2-(2,6-dioxopiperidin-3-yl)-5-(6-(piperidin-4-ylmethyl)-3,6-diazabicyclo[3.1.1]heptan-3-yl)isoindoline-1,3-dione;
2-(2,6-dioxopiperidin-3-yl)-5-fluoro-6-(6-(piperidin-4-ylmethyl)-3,6-diazabicyclo[3.1.1]heptan-3-yl)isoindoline-1,3-dione;
2-(2,6-dioxopiperidin-3-yl)-5-(3-(piperidin-4-ylmethyl)-3,8-diazabicyclo[3.2.1]octan-8-yl)isoindoline-1,3-dione;
2-(2,6-dioxopiperidin-3-yl)-5-fluoro-6-(3-(piperidin-4-ylmethyl)-3,8-diazabicyclo[3.2.1]octan-8-yl)isoindoline-1,3-dione;
2-(2,6-dioxopiperidin-3-yl)-5-(8-(piperidin-4-ylmethyl)-3,8-diazabicyclo[3.2.1]octan-3-yl)isoindoline-1,3-dione;
2-(2,6-dioxopiperidin-3-yl)-5-fluoro-6-(8-(piperidin-4-ylmethyl)-3,8-diazabicyclo[3.2.1]octan-3-yl)isoindoline-1,3-dione;
2-(2,6-dioxopiperidin-3-yl)-5-(5-(piperidin-4-ylmethyl)-2,5-diazabicyclo[2.2.1]heptan-2-yl)isoindoline-1,3-dione;
2-(2,6-dioxopiperidin-3-yl)-5-fluoro-6-(5-(piperidin-4-ylmethyl)-2,5-diazabicyclo[2.2.1]heptan-2-yl)isoindoline-1,3-dione;
2-(2,6-dioxopiperidin-3-yl)-5-((1R,4R)-5-(piperidin-4-ylmethyl)-2,5-diazabicyclo[2.2.1]heptan-2-yl)isoindoline-1,3-dione;
2-(2,6-dioxopiperidin-3-yl)-5-fluoro-6-((1R,4R)-5-(piperidin-4-ylmethyl)-2,5-diazabicyclo[2.2.1]heptan-2-yl)isoindoline-1,3-dione;
2-(2,6-dioxopiperidin-3-yl)-5-((1S,4S)-5-(piperidin-4-ylmethyl)-2,5-diazabicyclo[2.2.1]heptan-2-yl)isoindoline-1,3-dione;
2-(2,6-dioxopiperidin-3-yl)-5-fluoro-6-((1S,4S)-5-(piperidin-4-ylmethyl)-2,5-diazabicyclo[2.2.1]heptan-2-yl)isoindoline-1,3-dione;
2-(2,6-dioxopiperidin-3-yl)-5-(5-(piperidin-4-ylmethyl)-2,5-diazabicyclo[2.2.2]octan-2-yl)isoindoline-1,3-dione;
2-(2,6-dioxopiperidin-3-yl)-5-fluoro-6-(5-(piperidin-4-ylmethyl)-2,5-diazabicyclo[2.2.2]octan-2-yl)isoindoline-1,3-dione;
2-(2,6-dioxopiperidin-3-yl)-5-(4-(piperidin-4-ylmethyl)piperazin-1-yl-2,2,3,3,5,5,6,6-d8)isoindoline-1,3-dione;
2-(2,6-dioxopiperidin-3-yl)-5-fluoro-6-(4-(piperidin-4-ylmethyl)piperazin-1-yl-2,2,3,3,5,5,6,6-d8)isoindoline-1,3-dione;
2-(2,6-dioxopiperidin-3-yl)-4-fluoro-6-(4-(piperidin-4-ylmethyl)piperazin-1-yl-2,2,3,3,5,5,6,6-d8)isoindoline-1,3-dione;
2-(2,6-dioxopiperidin-3-yl)-4-fluoro-5-(4-(piperidin-4-ylmethyl)piperazin-1-yl-2,2,3,3,5,5,6,6-d8)isoindoline-1,3-dione;
2-(2,6-dioxopiperidin-3-yl)-4,6-difluoro-5-(4-(piperidin-4-ylmethyl)piperazin-1-yl-2,2,3,3,5,5,6,6-d8)isoindoline-1,3-dione;
2-(2,6-dioxopiperidin-3-yl)-4,7-difluoro-5-(4-(piperidin-4-ylmethyl)piperazin-1-yl-2,2,3,3,5,5,6,6-d8)isoindoline-1,3-dione;
2-(2,6-dioxopiperidin-3-yl)-4,5-difluoro-6-(4-(piperidin-4-ylmethyl)piperazin-1-yl-2,2,3,3,5,5,6,6-d8)isoindoline-1,3-dione;
2-(2,6-dioxopiperidin-3-yl)-4,5,7-trifluoro-6-(4-(piperidin-4-ylmethyl)piperazin-1-yl-2,2,3,3,5,5,6,6-d8)isoindoline-1,3-dione;
5-(2,6-dimethyl-4-(piperidin-4-ylmethyl)piperazin-1-yl)-2-(2,6-dioxopiperidin-3-yl)isoindoline-1,3-dione;
5-(2,6-dimethyl-4-(piperidin-4-ylmethyl)piperazin-1-yl)-2-(2,6-dioxopiperidin-3-yl)-6-fluoroisoindoline-1,3-dione;
5-(3,5-dimethyl-4-(piperidin-4-ylmethyl)piperazin-1-yl)-2-(2,6-dioxopiperidin-3-yl)-6-fluoroisoindoline-1,3-dione;
5-(3,5-dimethyl-4-(piperidin-4-ylmethyl)piperazin-1-yl)-2-(2,6-dioxopiperidin-3-yl)isoindoline-1,3-dione;
2-(2,6-dioxopiperidin-3-yl)-5-(1-(piperidin-4-ylmethyl)piperidin-4-yl)isoindoline-1,3-dione;
2-(2,6-dioxopiperidin-3-yl)-5-fluoro-6-(1-(piperidin-4-ylmethyl)piperidin-4-yl)isoindoline-1,3-dione;
2-(2,6-dioxopiperidin-3-yl)-5-((R)-3-((piperidin-4-ylmethyl)amino)piperidin-1-yl)isoindoline-1,3-dione;
2-(2,6-dioxopiperidin-3-yl)-5-((S)-3-((piperidin-4-ylmethyl)amino)piperidin-1-yl)isoindoline-1,3-dione;
2-(2,6-dioxopiperidin-3-yl)-5-(((S)-1-(piperidin-4-ylmethyl)piperidin-3-yl)amino)isoindoline-1,3-dione;
2-(2,6-dioxopiperidin-3-yl)-5-(((R)-1-(piperidin-4-ylmethyl)piperidin-3-yl)amino)isoindoline-1,3-dione;
5-(3-(azetidin-3-ylmethyl)-3,6-diazabicyclo[3.1.1]heptan-6-yl)-2-(2,6-dioxopiperidin-3-yl)isoindoline-1,3-dione;
5-(3-(azetidin-3-ylmethyl)-3,6-diazabicyclo[3.1.1]heptan-6-yl)-2-(2,6-dioxopiperidin-3-yl)-6-fluoroisoindoline-1,3-dione;
5-(6-(azetidin-3-ylmethyl)-3,6-diazabicyclo[3.1.1]heptan-3-yl)-2-(2,6-dioxopiperidin-3-yl)isoindoline-1,3-dione;
5-(6-(azetidin-3-ylmethyl)-3,6-diazabicyclo[3.1.1]heptan-3-yl)-2-(2,6-dioxopiperidin-3-yl)-6-fluoroisoindoline-1,3-dione;
5-(3-(azetidin-3-ylmethyl)-3,8-diazabicyclo[3.2.1]octan-8-yl)-2-(2,6-dioxopiperidin-3-yl)isoindoline-1,3-dione;
5-(3-(azetidin-3-ylmethyl)-3,8-diazabicyclo[3.2.1]octan-8-yl)-2-(2,6-dioxopiperidin-3-yl)-6-fluoroisoindoline-1,3-dione;
5-(8-(azetidin-3-ylmethyl)-3,8-diazabicyclo[3.2.1]octan-3-yl)-2-(2,6-dioxopiperidin-3-yl)isoindoline-1,3-dione;
2-(2,6-dioxopiperidin-3-yl)-5-fluoro-6-(8-(piperidin-4-ylmethyl)-3,8-diazabicyclo[3.2.1]octan-3-5-(5-(azetidin-3-ylmethyl)-2,5-diazabicyclo[2.2.1]heptan-2-yl)-2-(2,6-dioxopiperidin-3-yl)isoindoline-1,3-dione;
5-(5-(azetidin-3-ylmethyl)-2,5-diazabicyclo[2.2.1]heptan-2-yl)-2-(2,6-dioxopiperidin-3-yl)-6-fluoroisoindoline-1,3-dione;
5-((1R,4R)-5-(azetidin-3-ylmethyl)-2,5-diazabicyclo[2.2.1]heptan-2-yl)-2-(2,6-dioxopiperidin-3-yl)isoindoline-1,3-dione;
5-((1R,4R)-5-(azetidin-3-ylmethyl)-2,5-diazabicyclo[2.2.1]heptan-2-yl)-2-(2,6-dioxopiperidin-3-yl)-6-fluoroisoindoline-1,3-dione;
5-((1S,4S)-5-(azetidin-3-ylmethyl)-2,5-diazabicyclo[2.2.1]heptan-2-yl)-2-(2,6-dioxopiperidin-3-yl)isoindoline-1,3-dione;
5-((1S,4S)-5-(azetidin-3-ylmethyl)-2,5-diazabicyclo[2.2.1]heptan-2-yl)-2-(2,6-dioxopiperidin-3-yl)-6-fluoroisoindoline-1,3-dione;
5-(5-(azetidin-3-ylmethyl)-2,5-diazabicyclo[2.2.2]octan-2-yl)-2-(2,6-dioxopiperidin-3-yl)isoindoline-1,3-dione;
5-(5-(azetidin-3-ylmethyl)-2,5-diazabicyclo[2.2.2]octan-2-yl)-2-(2,6-dioxopiperidin-3-yl)-6-fluoroisoindoline-1,3-dione;
5-(4-(azetidin-3-ylmethyl)piperazin-1-yl-2,2,3,3,5,5,6,6-d8)-2-(2,6-dioxopiperidin-3-yl)isoindoline-1,3-dione;
5-(4-(azetidin-3-ylmethyl)piperazin-1-yl-2,2,3,3,5,5,6,6-d8)-2-(2,6-dioxopiperidin-3-yl)-6-fluoroisoindoline-1,3-dione;
6-(4-(azetidin-3-ylmethyl)piperazin-1-yl-2,2,3,3,5,5,6,6-d8)-2-(2,6-dioxopiperidin-3-yl)-4-fluoroisoindoline-1,3-dione;
5-(4-(azetidin-3-ylmethyl)piperazin-1-yl-2,2,3,3,5,5,6,6-d8)-2-(2,6-dioxopiperidin-3-yl)-4-fluoroisoindoline-1,3-dione;
5-(4-(azetidin-3-ylmethyl)piperazin-1-yl-2,2,3,3,5,5,6,6-d8)-2-(2,6-dioxopiperidin-3-yl)-4,6-difluoroisoindoline-1,3-dione;
5-(4-(azetidin-3-ylmethyl)piperazin-1-yl-2,2,3,3,5,5,6,6-d8)-2-(2,6-dioxopiperidin-3-yl)-4,7-difluoroisoindoline-1,3-dione;
6-(4-(azetidin-3-ylmethyl)piperazin-1-yl-2,2,3,3,5,5,6,6-d8)-2-(2,6-dioxopiperidin-3-yl)-4,5-difluoroisoindoline-1,3-dione;
5-(4-(azetidin-3-ylmethyl)piperazin-1-yl-2,2,3,3,5,5,6,6-d8)-2-(2,6-dioxopiperidin-3-yl)-4,6,7-trifluoroisoindoline-1,3-dione;
5-(4-(azetidin-3-ylmethyl)-2,6-dimethylpiperazin-1-yl)-2-(2,6-dioxopiperidin-3-yl)isoindoline-1,3-dione;
5-(4-(azetidin-3-ylmethyl)-2,6-dimethylpiperazin-1-yl)-2-(2,6-dioxopiperidin-3-yl)-6-fluoroisoindoline-1,3-dione;
5-(4-(azetidin-3-ylmethyl)-3,5-dimethylpiperazin-1-yl)-2-(2,6-dioxopiperidin-3-yl)-6-fluoroisoindoline-1,3-dione;
5-(4-(azetidin-3-ylmethyl)-3,5-dimethylpiperazin-1-yl)-2-(2,6-dioxopiperidin-3-yl)isoindoline-1,3-dione;
5-(1-(azetidin-3-ylmethyl)piperidin-4-yl)-2-(2,6-dioxopiperidin-3-yl)isoindoline-1,3-dione;
5-(1-(azetidin-3-ylmethyl)piperidin-4-yl)-2-(2,6-dioxopiperidin-3-yl)-6-fluoroisoindoline-1,3-dione;
5-((R)-3-((azetidin-3-ylmethyl)amino)piperidin-1-yl)-2-(2,6-dioxopiperidin-3-yl)isoindoline-1,3-dione;
5-((S)-3-((azetidin-3-ylmethyl)amino)piperidin-1-yl)-2-(2,6-dioxopiperidin-3-yl)isoindoline-1,3-dione;
5-(((S)-1-(azetidin-3-ylmethyl)piperidin-3-yl)amino)-2-(2,6-dioxopiperidin-3-yl)isoindoline-1,3-dione; and
5-(((R)-1-(azetidin-3-ylmethyl)piperidin-3-yl)amino)-2-(2,6-dioxopiperidin-3-yl)isoindoline-1,3-dione.

20. A compound of Formula (III)
PBM-R¹—W¹-LIN—ULM Formula (III)
or salts, enantiomers, stereoisomers, solvates, isotopically enriched analogs, prodrugs, or polymorphs thereof,
wherein PBM-R¹—W¹— represents a targeting moiety capable of binding protein, ULM represents a E3 ubiquitin ligase ligand moiety, LIN is a linker moiety, and PBM-R¹—W¹— is covalently bonded to ULM through LIN;
ULM represents the structure of Formula (ULM): 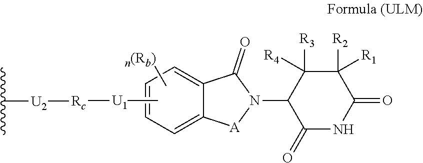
wherein A represents CO, CH₂ or CD₂;
R₁, R₂, R₃ and R₄ are the same or different and each independently represent hydrogen, deuterium, halogen, optionally deuterated C₁₋₆ alkyl, optionally deuterated C₁₋₆ alkoxy, or halogenated C₁₋₆ alkyl;
(R_{b})ₙ indicates that benzene ring is optionally substituted with n R_{b} groups, and each R_{b} are the same or different and each independently represent hydrogen, deuterium, halogen, hydroxy, mercapto, nitro, amino, cyano, optionally deuterated C₁₋₆ alkyl, optionally deuterated C₁₋₆ alkoxy, halogenated C₁₋₆ alkyl, C₂₋₆ alkenyl or C₂₋₆ alkynyl;
n represents an integer of 0, 1, 2 or 3;
U₁ and U₂ are the same or different and each independently represent a bond, N(R_{d}) or O, wherein R_{d} represents H or optionally deuterated C₁₋₆ alkyl;
R_{c} represents optionally substituted nitrogen-containing monocyclic heterocyclylene or optionally substituted nitrogen-containing bridged heterocyclylene;
LIN represents a bond, optionally substituted methylene, or optionally substituted linear or branched C₂₋₃₀ alkylene, wherein one or more groups Rₑ and/or one or more groups R_{f} and/or any combination of one or more groups Rₑ and R_{f} are optionally inserted into the backbone carbon chain of the linear or branched C₂₋₃₀ alkylene group, wherein carbon-carbon bond between one or more pairs of adjacent carbon atoms in the backbone carbon chain is interrupted by the group Rₑ, R_{f}, or a combination of Rₑ and R_{f}; wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl, and in case that two or more groups Rₑ are inserted into the backbone carbon chain of the linear or branched C₂₋₃₀ alkylene group, the two or more groups Rₑ are not directly connected to each other; and wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano or any combination thereof;
W¹ is t interconnected rings W² and represented by the following formula: 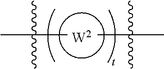
each ring W² are the same or different and each independently represent optionally substituted nitrogen-containing monocyclic heterocyclylene, optionally substituted nitrogen-containing bridged heterocyclylene, or optionally substituted cycloalkylene, and
t represents an integer of 1 or 2; and
R¹ represents:
a bond, —O—, optionally substituted C₁₋₆ alkylene (e.g., methylene), —N(R^{1a})—R^{2a}—*** (e.g., 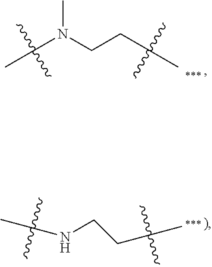
—O—R^{2a}—*** (e.g., 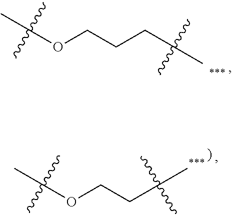 [SMILES: *CCCO* ; *CCO*]
—C(O)NH—R^{2a}—***, or —NHC(O)—R^{2a}—*** (e.g., 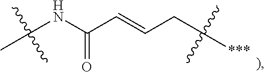
wherein symbol *** indicates the point of attachment to W¹, R^{1a} is H or C₁₋₆ alkyl, and R^{2a} is optionally substituted C₁₋₆ alkylene or optionally substituted C₁₋₆ alkenylene; or
C₆₋₁₀ aryl or heteroaryl containing one or two 5- to 7-membered rings and 1 to 4 heteroatoms selected from nitrogen, oxygen, and sulfur, wherein the C₆₋₁₀ aryl and heteroaryl are each optionally substituted with one or more R^{3a}, and each R^{3a} are the same or different and are each independently halogen, hydroxy, cyano, amino, nitro, C₁₋₆ alkyl, halogenated C₁₋₆ alkyl, C₁₋₆ alkoxy, or halogenated C₁₋₆ alkoxy;
wherein one or more hydrogens of the compound of Formula (III) are optionally replaced with deuterium;
wherein when the compound of Formula (III) is not deuterated, the following compounds are excluded: 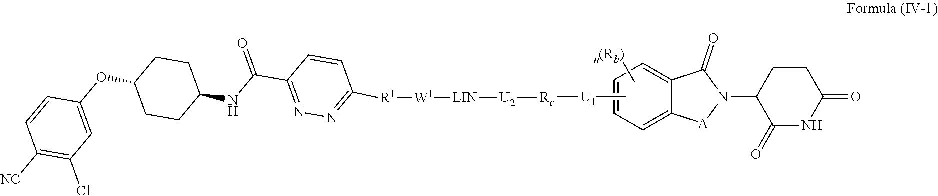
in Formula (IV-1), R¹ represents a bond, W¹ represents piperidinylene, LIN represents methylene, R_{c} represents piperazinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents fluoro, n represents an integer of 1, 2 or 3, and U₁ and U₂ are the same or different and each independently represent a bond, NH or O; or
in Formula (IV-1), R¹ represents a bond, W¹ represents piperazinylene, LIN represents methylene, R_{c} represents piperidinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents fluoro, n represents an integer of 1, 2 or 3, and U₁ and U₂ are the same or different and each independently represent a bond, NH or O; 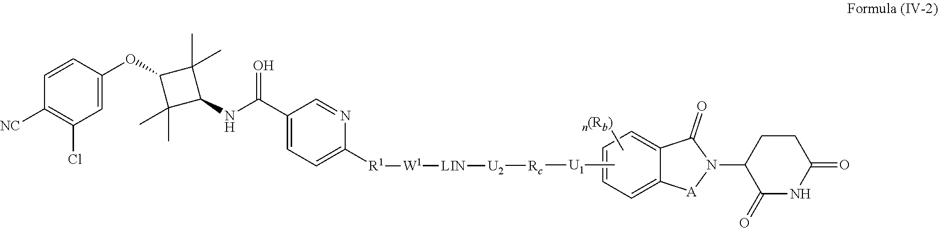
in Formula (IV-2), R¹ represents a bond, W¹ represents piperidinylene, LIN represents methylene, R_{c} represents piperazinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents fluoro, n represents an integer of 1, 2 or 3, and U₁ and U₂ are the same or different and each independently represent a bond, NH or O; or
in Formula (IV-2), R¹ represents a bond, W¹ represents piperazinylene, LIN represents methylene, R_{c} represents piperidinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents fluoro, and n represents an integer of 1, 2 or 3, and U₁ and U₂ are the same or different and each independently represent a bond, NH or O; 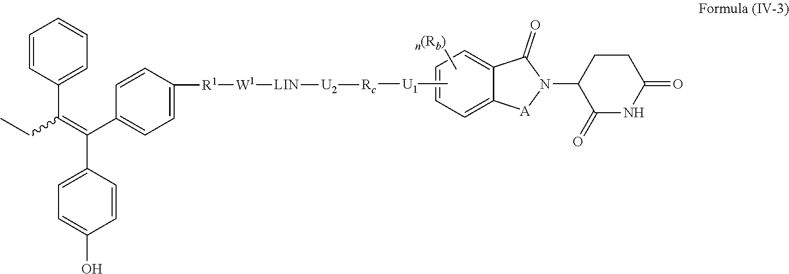
in Formula (IV-3), R¹ represents a bond or —O—CH₂—CH₂—, W¹ represents piperidinylene, LIN represents methylene, R_{c} represents piperazinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents hydrogen, and U₁ and U₂ are the same or different and each independently represent a bond, NH or O; or
in Formula (IV-3), R¹ represents a bond or —O—CH₂—CH₂—, W¹ represents piperazinylene, LIN represents methylene, R_{c} represents piperidinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents hydrogen, and U₁ and U₂ are the same or different and each independently represent a bond, NH or 0;
and 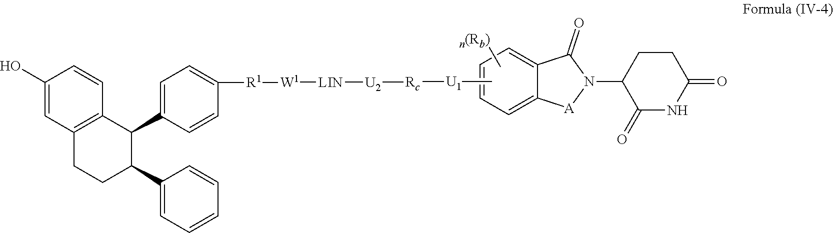
in Formula (IV-4), R¹ represents a bond or represents —O—CH₂—CH₂—, W¹ represents piperidinylene, LIN represents methylene, R_{c} represents piperazinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents hydrogen, and U₁ and U₂ are the same or different and each independently represent a bond, NH or O; or
in Formula (IV-4), R¹ represents a bond or represents —O—CH₂—CH₂—, W¹ represents piperazinylene, LIN represents methylene, R_{c} represents piperidinylene, 2,5-diazabicyclo[2.2.1]heptanylene, 8-azabicyclo[3.2.1]octanylene, or 9-ethyl-3-methyl-3,9-diazabicyclo[3.3.1]nonanylene, R_{b} represents hydrogen, and U₁ and U₂ are the same or different and each independently represent a bond, NH or O.

21. The compound of Formula (III) or salts, enantiomers, stereoisomers, solvates, isotopically enriched analogs, prodrugs, or polymorphs thereof as claimed in claim 20, wherein the PBM is a small molecule ligand targeting EGFR, CDK4/6, ALK, RET, FAK, BCR-ABL, BTK, AR, ER or BET.

22. The compound of Formula (III) or salts, enantiomers, stereoisomers, solvates, isotopically enriched analogs, prodrugs, or polymorphs thereof as claimed in claim 20, wherein (i) the PBM comprises the structures of the following formula: 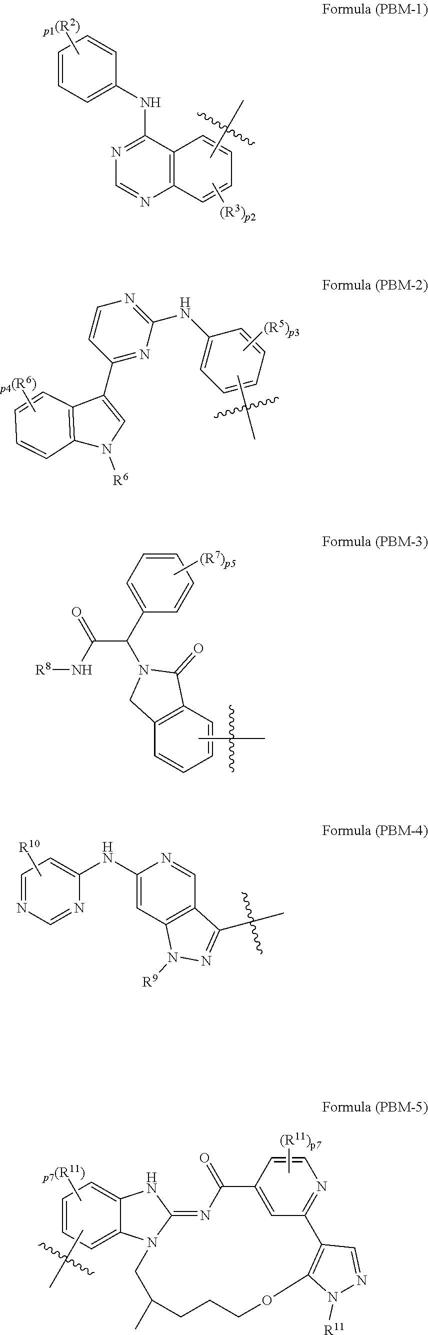 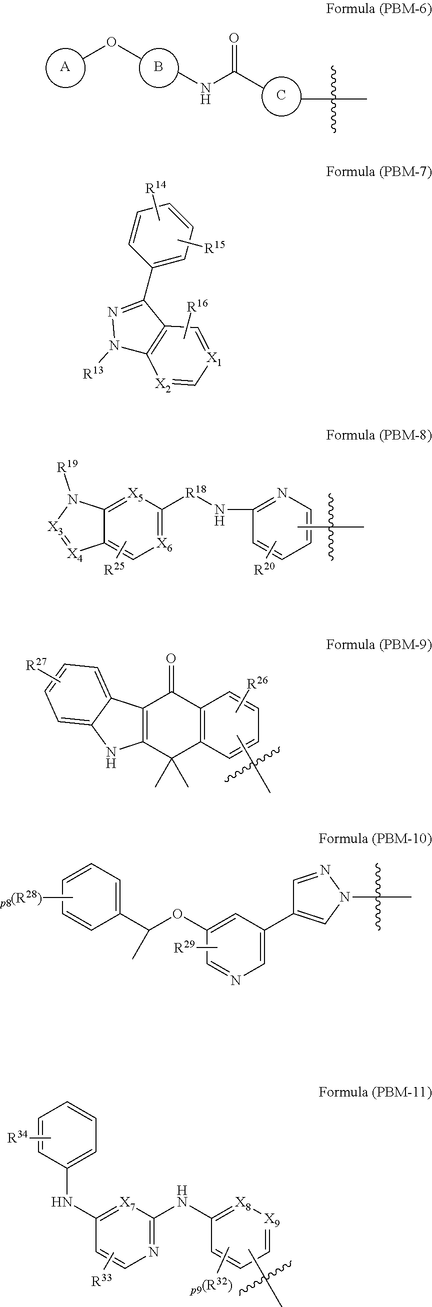 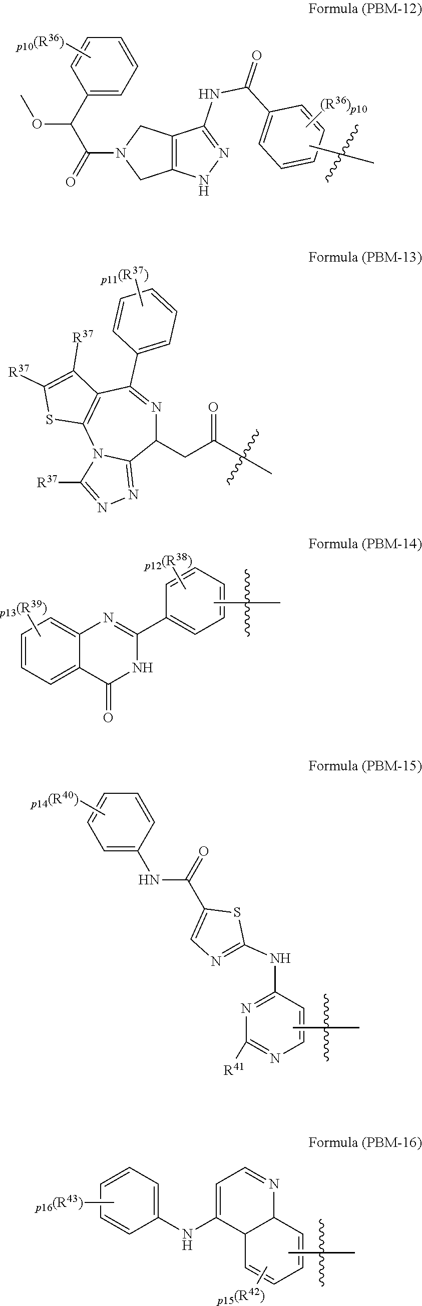 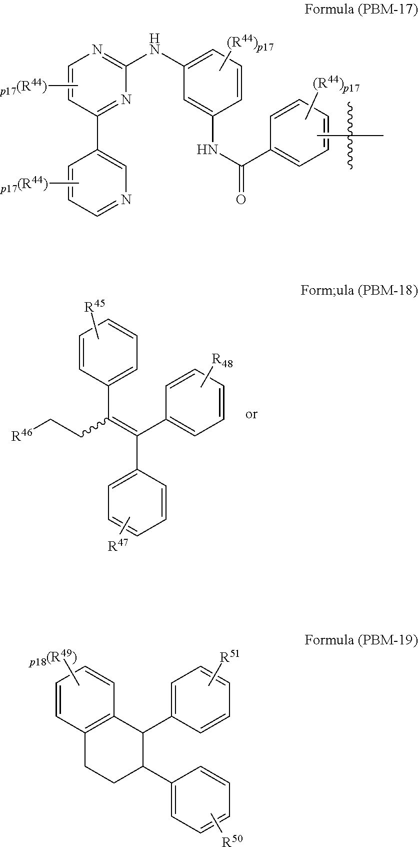
wherein
(R²)ₚ₁ indicates that the benzene ring in Formula (PBM-1) is substituted with p1 R³, and each R² are the same or different and each independently represent halogen or halogenated C₁₋₆ alkyl;
(R³)_{pz} indicates that the quinazoline ring in Formula (PBM-1) is substituted with p2 R³, and each R³ are the same or different and each independently represent C₁₋₁₀ alkyl, deuterated C₁₋₁₀ alkyl, C₁₋₁₀ alkoxy, heterocyclyloxy, or —NHC(O)R⁴, wherein R⁴ is C₁₋₁₀ alkyl or C₂₋₁₀ alkenyl, e.g., R³ represents methyl, methoxy, 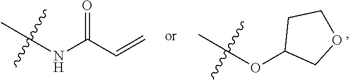
such as; 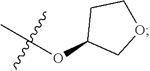 [SMILES: *O[C@H]1CCOC1]
p1 represents an integer of 1, 2, 3, 4 or 5;
p2 represents an integer of 1, 2 or 3;
(R⁵)ₚ₃ indicates that the benzene ring in Formula (PBM-2) is substituted with p3 R⁵, and each R⁵ are the same or different and each independently represent C₁₋₁₀ alkyl, deuterated C₁₋₁₀ alkyl, C₁₋₁₀ alkoxy, or —NHC(O)R⁴, wherein R⁴ is C₁₋₁₀ alkyl, deuterated C₁₋₁₀ alkyl or C₂₋₁₀ alkenyl, e.g., R⁵ represents methyl, methoxy or 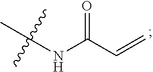 [SMILES: *NC(=O)C=C]
p3 represents an integer of 1, 2, 3 or 4;
(R⁶)ₚ₄ indicates that the 1H-indole ring in Formula (PBM-2) is substituted with p4 R⁶, and each R⁶ are the same or different and each independently represent C₁₋₁₀ alkyl or deuterated C₁₋₁₀ alkyl;
p4 represents an integer of 0, 1, 2 or 3;
(R⁷)ₚ₅ indicates that the benzene ring in Formula (PBM-3) is substituted with p5 R⁷, and each R⁷ are the same or different and each independently represent halogen, hydroxy, C₁₋₆ alkyl, halogenated C₁₋₆ alkyl, deuterated C₁₋₆ alkyl, NO₂, NH₂, or cyano;
p5 represents an integer of 2, 3, 4 or 5;
R⁸ represents C₆₋₁₀ aryl or heteroaryl containing one or two 5- to 7-membered rings and 1 to 4 heteroatoms selected from nitrogen, oxygen, and sulfur, wherein the C₆₋₁₀ aryl and heteroaryl are each optionally substituted with one or more R^{4a}, and each R^{4a} are the same or different and are each independently halogen, hydroxy, cyano, amino, nitro, C₁₋₆ alkyl, halogenated C₁₋₆ alkyl, deuterated C₁₋₆ alkyl, C₁₋₆ alkoxy, or halogenated C₁₋₆ alkoxy;
R⁹ represents C₁₋₁₀ alkyl (e.g., 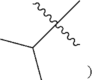 [SMILES: *C(C)C]
or deuterated C₁₋₁₀ alkyl;
R¹⁰ represents 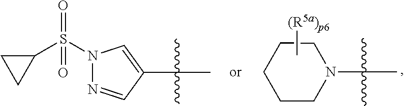
wherein (R^{5a})ₚ₆ indicates that the piperidine ring is substituted with p6 R^{5a}, and each R^{5a} are the same or different and each independently represent deuterium, halogen, hydroxy, C₁₋₆ alkyl, halogenated C₁₋₆ alkyl, deuterated C₁₋₆ alkyl, C₁₋₆ alkoxy, deuterated C₁₋₆ alkoxy, NO₂, NH₂, or cyano, p6 represents an integer of 1, 2, 3, 4 or 5;
(R¹¹)ₚ₇ indicates that the benzene ring and the pyridine ring in Formula (PBM-5) are each optionally substituted with p7 R¹¹, and each R¹¹ are the same or different and each independently represent halogen, C₁₋₆ alkyl, halogenated C₁₋₆ alkyl, or deuterated C₁₋₆ alkyl, p7 represents an integer of 0, 1, 2 or 3;
ring A in Formula (PBM-6) represents C₆₋₁₀ aryl or 5- to 20-membered monocyclic or bicyclic heteroaryl containing from 1 to 4 heteroatoms selected from the group consisting of oxygen, nitrogen, and sulfur, and the ring A is optionally substituted with one or more R^{6a}, and each R^{6a} are the same or different and each independently represent deuterium, halogen, hydroxy, cyano, amino, nitro, C₁₋₆ alkyl, halogenated C₁₋₆ alkyl, deuterated C₁₋₆ alkyl, C₁₋₆ alkoxy, halogenated C₁₋₆ alkoxy, C₂₋₆ alkenyl, or C₂₋₆ alkynyl;
ring B in Formula (PBM-6) represents C₃₋₁₅ cycloalkyl or 3- to 20-membered heterocyclyl containing from 1 to 4 heteroatoms selected from the group consisting of oxygen, nitrogen, and sulfur, and the ring B is optionally substituted with one or more R^{7a}, and each R^{7a} are the same or different and each independently represent deuterium, halogen, C₁₋₆ alkyl, halogenated C₁₋₆ alkyl, deuterated C₁₋₆ alkyl, C₁₋₆ alkoxy, or halogenated C₁₋₆ alkoxy;
ring C in Formula (PBM-6) represents 5- to 20-membered monocyclic or bicyclic heteroaryl containing from 1 to 4 heteroatoms selected from the group consisting of oxygen, nitrogen, and sulfur, and the ring C is optionally substituted with one or more R^{8a}, and each R^{8a} are the same or different and each independently represent deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
X₁ in Formula (PBM-7) represents N or CR¹², wherein R¹² is hydrogen or represents a bond, and X₂ represents CH or N;
R¹³ is hydrogen or represents a bond, wherein when R¹³ represents a bond, X₁ represents N or CH, and the nitrogen atom in the ring connected to R¹³ is directly connected to R¹; or when R¹³ is hydrogen, X₁ represents CR¹², wherein R¹² represents a bond, and the carbon atom in the ring connected to R¹² is directly connected to R¹;
R¹⁴ represents —O-aryl, —O-heteroaryl, —C₁₋₃-alkylene-NHC(O)-heteroaryl, or —C₁₋₃-alkylene-C(O)NH-heteroaryl, wherein the aryl and heteroaryl are each independently optionally substituted with one or more R^{9a}, and each R^{9a} are the same or different and each independently represent deuterium, halogen, hydroxy, cyano, amino, C₁₋₆ alkyl, halogenated C₁₋₆ alkyl, deuterated C₁₋₆ alkyl, C₁₋₆ alkoxy, or halogenated C₁₋₆ alkoxy;
R¹⁵ represents deuterium, hydrogen, halogen, C₁₋₆ alkyl, halogenated C₁₋₆ alkyl, deuterated C₁₋₆ alkyl or —NHC(O)R¹⁷, wherein R¹⁷ is C₁₋₁₀ alkyl or C₂₋₁₀ alkenyl, e.g., R¹⁵ represents hydrogen, methyl, or 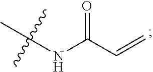 [SMILES: *NC(=O)C=C]
R¹⁶ represents hydrogen, cyano, or amino;
X₃ in Formula (PBM-8) represents CR²¹ or a fragment 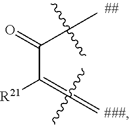
wherein symbol ## indicates the point of attachment to N atom adjacent to X₃, symbol ### indicates the point of attachment to X₄, and R²¹ represents deuterium, hydrogen, halogen, C₁₋₆ alkyl, halogenated C₁₋₆ alkyl, deuterated C₁₋₆ alkyl, —C(O)—C₁₋₆ alkyl, —C(O)-deuterated C₁₋₆ alkyl, or —C(O)NR²²R²³, wherein R²² and R²³ are the same or different and each independently represent hydrogen, C₁₋₆ alkyl or deuterated C₁₋₆ alkyl, and R²² and R²³ are not simultaneously hydrogen;
X₄ in Formula (PBM-8) represents N or CR²⁴, wherein R²⁴ represents hydrogen, deuterium, halogen, C₁₋₆ alkyl, deuterated C₁₋₆ alkyl or halogenated C₁₋₆ alkyl;
X₅ and X₆ each independently represent CH or N;
R¹⁸ represents a bond or optionally substituted heteroarylene (e.g., halogenated heteroarylene);
R¹⁹ represents hydrogen, C₁₋₆ alkyl, deuterated C₁₋₆ alkyl, halogenated C₁₋₆ alkyl, or optionally substituted C₃₋₇ cycloalkyl;
R²⁰ and R²⁵ are the same or different and each independently represent hydrogen, halogen, C₁₋₆ alkyl, deuterated C₁₋₆ alkyl or halogenated C₁₋₆ alkyl;
R²⁶ represents hydrogen, halogen, C₁₋₆ alkyl, deuterated C₁₋₆ alkyl or halogenated C₁₋₆ alkyl;
R²⁷ represents hydrogen, cyano, halogen, C₁₋₆ alkyl, deuterated C₁₋₆ alkyl or halogenated C₁₋₆ alkyl;
(R²⁸)ₚ₈ indicates that the benzene ring in Formula (PBM-10) is substituted with p8 R²⁸ substituents, and each R²⁸ are the same or different and each independently represent halogen, C₁₋₆ alkyl, deuterated C₁₋₆ alkyl or halogenated C₁₋₆ alkyl;
p8 represents an integer of 1, 2, 3 or 4;
R²⁹ represents NR³⁰R³¹, wherein R³⁰ and R³¹ are the same or different and each independently represent hydrogen, C₁₋₆ alkyl, or deuterated C₁₋₆ alkyl;
X₇, X₈ and X₉ in Formula (PBM-11) are the same or different and each independently represent N or CH;
(R³²)ₚ₉ indicates that the 6-membered ring containing X₈ and X₉ in Formula (PBM-11) is optionally substituted with p9 R³² groups, and each R³² are the same or different and each independently represent halogen, C₁₋₆ alkoxy, C₁₋₆ alkyl, deuterated C₁₋₆ alkyl or halogenated C₁₋₆ alkyl;
p9 represents an integer of 0, 1, 2, 3 or 4;
R³³ represents halogen, C₁₋₆ alkoxy, C₁₋₆ alkyl, deuterated C₁₋₆ alkyl or halogenated C₁₋₆ alkyl;
R³⁴ represents —C(O)NHR³⁵, —NHC(O)—R³⁵, —S(O)₂NHR³⁵, —S(O)₂R³⁵ or —P(O)(R³⁵)₂, wherein each R³⁵ are the same or different and each independently represent C₁₋₆ alkyl or deuterated C₁₋₆ alkyl;
(R³⁶)ₚ₁₀ indicates that the benzene ring in Formula (PBM-12) is optionally substituted with p10 R³⁶ groups, and each R³⁶ are the same or different and each independently represent halogen, C₁₋₆ alkoxy, C₁₋₆ alkyl, deuterated C₁₋₆ alkyl or halogenated C₁₋₆ alkyl;
p10 represents an integer of 0, 1, 2, 3 or 4;
(R³⁷)ₚ₁₁ indicates that the benzene ring in Formula (PBM-13) is substituted with p11 R³⁷ groups, and each R³⁷ are the same or different and each independently represent halogen, C₁₋₆ alkoxy, C₁₋₆ alkyl, deuterated C₁₋₆ alkyl or halogenated C₁₋₆ alkyl;
p11 represents an integer of 0, 1, 2, 3 or 4;
(R³⁸)ₚ₁₂ indicates that the benzene ring in Formula (PBM-14) is substituted with p12 R³⁸ groups, and each R³⁸ are the same or different and each independently represent halogen, C₁₋₆ alkoxy, C₁₋₆ alkyl, deuterated C₁₋₆ alkyl or halogenated C₁₋₆ alkyl;
p12 represents an integer of 0, 1, 2, 3 or 4;
(R³⁹)P₁₃ indicates that the 4-oxo-3,4-dihydroquinazoline ring in Formula (PBM-14) is substituted with p13 R³⁹ groups, and each R³⁹ are the same or different and each independently represent halogen, C₁₋₆ alkoxy, C₁₋₆ alkyl, deuterated C₁₋₆ alkyl or halogenated C₁₋₆ alkyl;
p13 represents an integer of 1, 2, 3 or 4;
(R⁴⁰)ₚ₁₄ indicates that the benzene ring in Formula (PBM-15) is substituted with p14 R⁴⁰ groups, and each R⁴⁰ are the same or different and each independently represent halogen, C₁₋₆ alkoxy, C₁₋₆ alkyl, deuterated C₁₋₆ alkyl or halogenated C₁₋₆ alkyl;
p14 represents an integer of 1, 2, 3 or 4;
R⁴¹ represents halogen, C₁₋₆ alkyl, deuterated C₁₋₆ alkyl or halogenated C₁₋₆ alkyl;
(R⁴²)ₚ₁₅ indicates that the quinoline ring in Formula (PBM-16) is substituted with p15 R⁴² groups, and each R⁴² are the same or different and each independently represent halogen, cyano, C₁₋₆ alkoxy, C₁₋₆ alkyl, deuterated C₁₋₆ alkyl or halogenated C₁₋₆ alkyl;
p15 represents an integer of 2, 3 or 4;
(R⁴³)ₚ₁₆ indicates that the benzene ring in Formula (PBM-16) is substituted with p16 R⁴³ groups, and each R⁴³ are the same or different and each independently represent halogen, C₁₋₆ alkoxy, C₁₋₆ alkyl, deuterated C₁₋₆ alkyl or halogenated C₁₋₆ alkyl;
p16 represents an integer of 3 or 4;
(R⁴⁴)P₁₇ indicates that the benzene, pyrimidine and pyridine rings in Formula (PBM-17) are each optionally substituted with p17 R⁴⁴ groups, and each R⁴⁴ are the same or different and each independently represent halogen, C₁₋₆ alkoxy, C₁₋₆ alkyl, deuterated C₁₋₆ alkyl or halogenated C₁₋₆ alkyl;
p17 represents an integer of 0, 1, 2, 3 or 4;
one of R⁴⁵, R⁴⁶, R⁴⁷ and R⁴⁸ represents a bond, and the remaining are the same or different and each independently represent hydrogen, halogen, or hydroxy, wherein when one of R⁴⁵, R⁴⁶, R⁴⁷ and R⁴⁸ represents a bond, the benzene ring or methylene in Formula (PBM-18) connected to the bond is directly connected to R¹;
symbol “ [symbol] ” connected to double bond in Formula (PBM-18) represents a bond in stereo-configuration (cis or trans configuration);
(R⁴⁹)ₚ₁₈ indicates that 1,2,3,4-tetrahydronaphthalene ring in Formula (PBM-19) is substituted with p18 R⁴⁹ groups, and each R⁴⁹ are the same or different and each independently represent hydrogen, hydroxy, halogen, C₁₋₆ alkyl, deuterated C₁₋₆ alkyl or halogenated C₁₋₆ alkyl;
p18 represents an integer of 0, 1, 2, 3 or 4; and
one of R⁵⁰ and R⁵¹ represents a bond, and another represents hydrogen, halogen, C₁₋₆ alkyl, deuterated C₁₋₆ alkyl or halogenated C₁₋₆ alkyl, wherein when one of R⁵⁰ and R⁵¹ represents a bond, the benzene ring in Formula (PBM-19) connected to the bond is directly connected to R¹;
and/or
(ii) the R¹ represents the structures of the following formula:
a bond, —O—, optionally substituted C₁₋₆ alkylene (e.g., methylene, ethylene, propylene, isopropylene, butylene, tert-butylene, pentylene and hexylene), —N(R^{1a})—R^{2a}—*** (e.g., —N(CH₃)—CH₂CH₂—***, —NH—CH₂CH₂—***), —O—R^{2a}—*** (e.g., —O—(CH₂)₃—***, —O—CH₂—*** and —O—(CH₂)₂—***), —C(O)NH—R^{2a}—*** (e.g., —C(O)NH—(CH₂)₂—***, —C(O)NH—(CH₂)₃—***, —C(O)NH—CH═CH—CH₂—***, and —C(O)NH—CH₂—CH═CH—CH₂—***), or —NHC(O)—R^{2a}—*** (e.g., —NHC(O)—CH═CH—CH₂—***, —NHC(O)—CH₂—CH═CH—CH₂—***, —NHC(O)—(CH₂)₂—***, and —NHC(O)—(CH₂)₃—***), wherein symbol *** indicates the point of attachment to W¹, R^{1a} is hydrogen or C₁₋₆ alkyl, and R^{2a} is optionally substituted C₁₋₆ alkylene or optionally substituted C₂₋₆ alkenylene; or
phenylene or naphthylene, wherein the phenylene or naphthylene is independently optionally substituted with one or more R^{3a}, and each R^{3a} are the same or different and each independently represent halogen, hydroxy, cyano, amino, nitro, C₁₋₆ alkyl, halogenated C₁₋₆ alkyl, C₁₋₆ alkoxy, or halogenated C₁₋₆ alkoxy; or
furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzisoxazolylene, benzothiazolylene, benzisothiazolylene, benzotriazolylene, benzo[2,1,3]oxadiazolylene, benzo[2,1,3]thiadiazolylene, benzo[1,2,3]thiadiazolylene, quinolinylene, isoquinolinylene, naphthyridinylene, cinnolinylene, quinazolinylene, quinoxalinylene, phthalazinylene, pyrazolo[1,5-a]pyridylene, pyrazolo[1,5-a]pyrimidinylene, imidazo[1,2-a]pyridylene, 1H-pyrrolo[3,2-b]pyridylene, 1H-pyrrolo[2,3-b]pyridylene, 4H-fluoro[3,2-b]pyrrolylene, pyrrolo[2,1-b]thiazolylene, and imidazo[2,1-b]thiazolylene, with each group being independently optionally substituted with one or more R^{3a}, and each R^{3a} are the same or different and are each independently halogen, hydroxy, cyano, amino, nitro, C₁₋₆ alkyl, halogenated C₁₋₆ alkyl, C₁₋₆ alkoxy, or halogenated C₁₋₆ alkoxy;
and/or
(iii) W¹ is t interconnected rings W² and represented by the following formula: 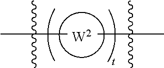
wherein each ring W² are the same or different and each independently represent the following groups:
monocyclic C₃₋₁₀ heterocyclylene group containing one nitrogen atom and optionally a heteroatom selected from nitrogen, oxygen, and sulfur, e.g., piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene or diazacyclooctylene; or
C₅₋₂₀ bridged heterocyclylene group containing one nitrogen atom and optionally a heteroatom selected from nitrogen, oxygen, and sulfur, e.g., azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene or diazabicyclo[2.2.2]octanylene, such as the following groups: 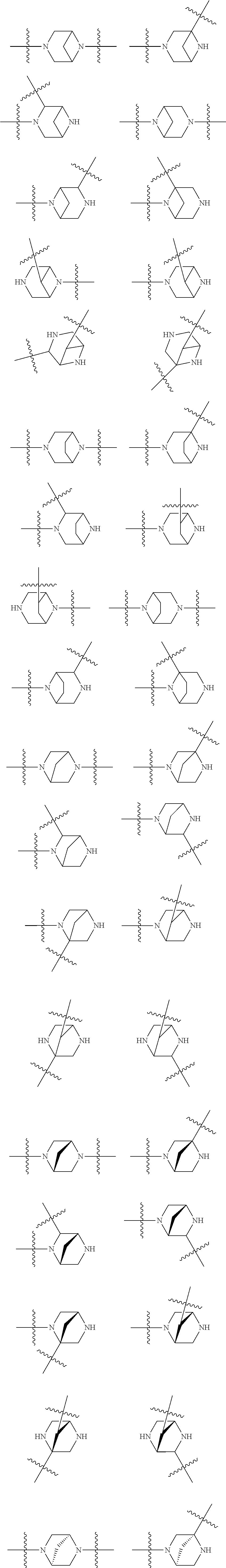 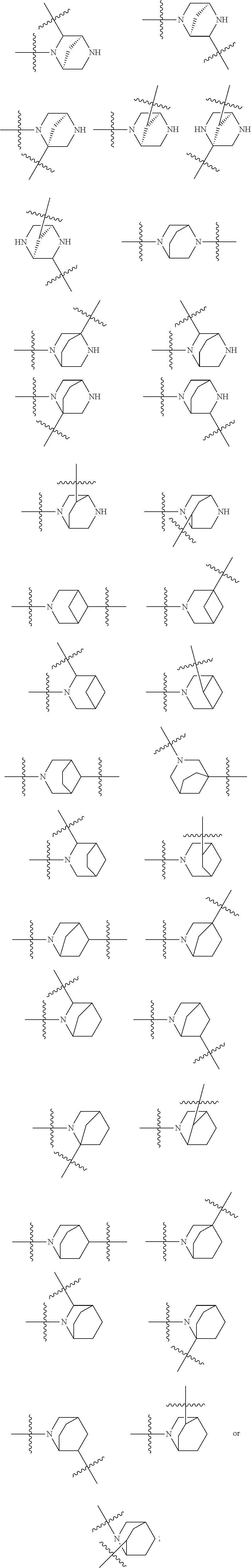
C₃₋₁₅ cycloalkylene, e.g., cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, C₅₋₁₅ spiro-cycloalkylene, adamantanylene, noradamantanylene, bornylene, bicyclo[2.2.1]heptylene or bicyclo[2.2.1]heptenylene;
wherein the monocyclic C₃₋₁₀ heterocyclylene, C₅₋₂₀ bridged heterocyclylene, and C₃₋₁₅ cycloalkylene are each independently optionally substituted with one or more substituents selected from the group consisting of deuterium, hydroxy, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof; and
t represents an integer of 1 or 2;
and/or
(iv) the ULM represents the structures of Formula (ULM-1), (ULM-2), (ULM-3), (ULM-4), (ULM-5), (ULM-6), (ULM-7), (ULM-8), (ULM-9), or (ULM-10): 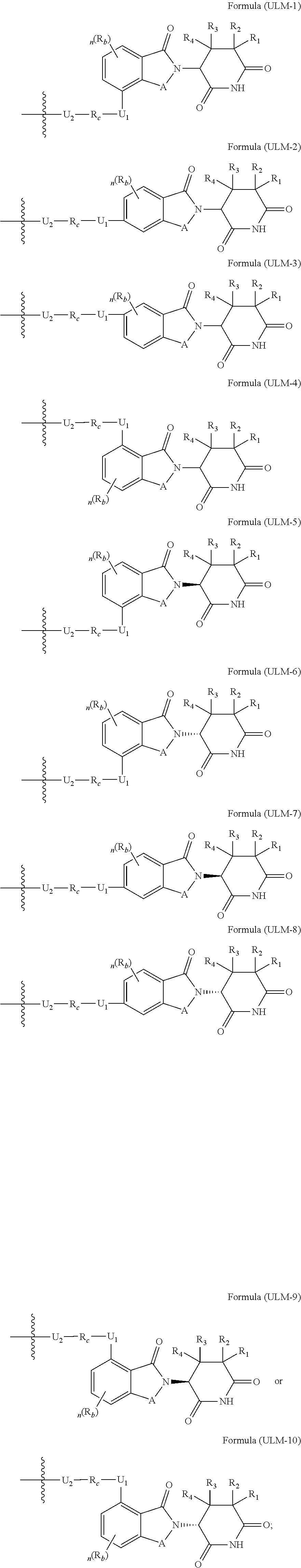
wherein A represents CO, CH₂ or CD₂;
R₁, R₂, R₃ and R₄ are the same or different and each independently represent hydrogen, deuterium, halogen, optionally deuterated C₁₋₆ alkyl, optionally deuterated C₁₋₆ alkoxy, or halogenated C₁₋₆ alkyl;
(R_{b})ₙ indicates that benzene ring is optionally substituted with n R_{b} groups, and each R_{b} are the same or different and each independently represent hydrogen, deuterium, halogen, hydroxy, mercapto, nitro, amino, cyano, optionally deuterated C₁₋₆ alkyl, optionally deuterated C₁₋₆ alkoxy, halogenated C₁₋₆ alkyl, C₂₋₆ alkenyl or C₂₋₆ alkynyl;
n represents an integer of 0, 1, 2 or 3;
U₁ and U₂ are the same or different and each independently represent a bond, N(R_{d}) or O, wherein R_{d} represents H or optionally deuterated C₁₋₆ alkyl;
R_{c} represents monocyclic C₃₋₁₀ heterocyclylene group containing one nitrogen atom and optionally a heteroatom selected from nitrogen, oxygen, and sulfur, e.g., piperidinylene, piperazinylene, morpholinylene, azetidinylene, pyrrolidinylene, imidazolidylene, pyrazolidylene, oxazolidinylene, thiazolidinylene, thiomorpholinylene, azepanylene, diazacycloheptanylene, azacyclooctylene or diazacyclooctylene; or
R_{c} represents C₅₋₂₀ bridged heterocyclylene group containing one nitrogen atom and optionally a heteroatom selected from nitrogen, oxygen, and sulfur, e.g., azabicyclo[3.1.1]heptanylene, azabicyclo[2.2.1]heptanylene, azabicyclo[3.2.1]octanylene, azabicyclo[2.2.2]octanylene, diazabicyclo[3.1.1]heptanylene, diazabicyclo[2.2.1]heptanylene, diazabicyclo[3.2.1]octanylene or diazabicyclo[2.2.2]octanylene, such as the following groups: 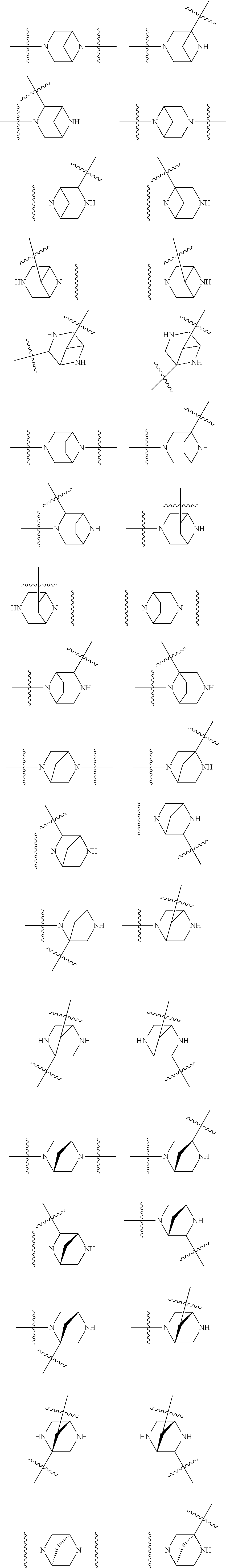 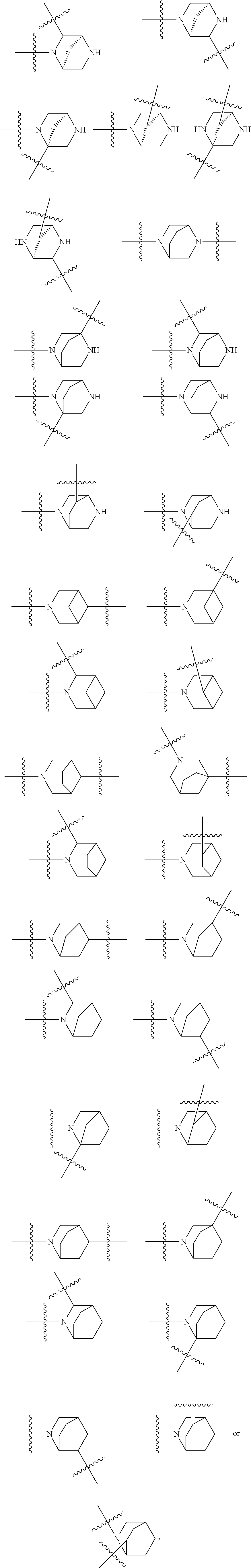
wherein the monocyclic C₃₋₁₀ heterocyclylene and C₅₋₂₀ bridged heterocyclylene are each independently optionally substituted with one or more substituents selected from the group consisting of deuterium, hydroxy, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH— or any combination thereof;
and/or
(v) the LIN represents the structures of the following formula:
#-C₁₋₃₀ alkylene-;
#-(C(R^{a1})(R^{a2}))ₙ₁—(Rₑ(C(R^{a3})(R^{a4}))ₙ₂)ₘ₁—;
#-(C(R^{a1})(R^{a2}))ₙ₁—(Rₑ(C(R^{a3})(R^{a4}))ₙ₂)ₘ₁—(Rₑ(C(R^{a5})(R^{a6}))ₙ₃)ₘ₂—;
#-(C(R^{a1})(R^{a2}))ₙ₁—(Rₑ(C(R^{a3})(R^{a4}))ₙ₂)ₘ₁—(Rₑ(C(R^{a5})(R^{a6}))ₙ₃)ₘ₂—(Rₑ(C(R^{a7})(R^{a8}))ₙ₄)ₘ₃—;
#-(C(R^{a1})(R^{a2}))ₙ₁—(R_{f}(C(R^{a3})(R^{a4}))ₙ₂)ₘ₁—;
#-(C(R^{a1})(R^{a2}))ₙ₁—(R_{f}(C(R^{a3})(R^{a4}))ₙ₂)ₘ₁—(R_{f}(C(R^{a5})(R^{a6}))ₙ₃)ₘ₂—;
#-(C(R^{a1})(R^{a2}))ₙ₁—(R_{f}(C(R^{a3})(R^{a4}))ₙ₂)ₘ₁—(R_{f}(C(R^{a5})(R^{a6}))ₙ₃)ₘ₂—(R_{f}(C(R^{a7})(R^{a8}))ₙ₄)ₘ₃—;
#-(C(R^{a1})(R^{a2}))ₙ₁—(Rₑ—R_{f}—(C(R^{a3})(R^{a4}))ₙ₂)ₘ₁—;
#-(C(R^{a1})(R^{a2}))ₙ₁—(Rₑ(C(R^{a3})(R^{a4}))ₙ₂)ₘ₁—(R_{f}(C(R^{a5})(R^{a6}))ₙ₃)ₘ₂—;
#-(C(R^{a1})(R^{a2}))ₙ₁—(R_{f}—Rₑ—(C(R^{a3})(R^{a4}))ₙ₂)ₘ₁—; or
#-(C(R^{a1})(R^{a2}))ₙ₁—(R_{f}(C(R^{a3})(R^{a4}))ₙ₂)ₘ₁—(Rₑ(C(R^{a5})(R^{a6}))ₙ₃)ₘ₂—;
wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl; wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano, or any combination thereof;
a hydrogen atom of one or more CH₂ groups of the C₁₋₃₀ alkylene group is optionally replaced with a substituent selected from the group consisting of deuterium, hydroxy, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH—, or any combination thereof;
R^{a1}, Rₐ₂, R^{3a}, R^{a4}, R^{a5}, R^{a6}, R^{a7}, and R^{a8} each independently represent H, deuterium, hydroxy, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH—, or any combination thereof;
symbol # indicates the point of attachment to U₂; and
n1, n2, n3, n4, m1, m2, and m3 each independently represent an integer of 1, 2, 3, 4, 5, 6, 7, 8, 9, 10, 11, 12, 13, 14, or 15.

23. The compound of Formula (III) or salts, enantiomers, stereoisomers, solvates, isotopically enriched analogs, prodrugs, or polymorphs thereof as claimed in claim 20, wherein (i) the PBM represents the structures of the following formula: 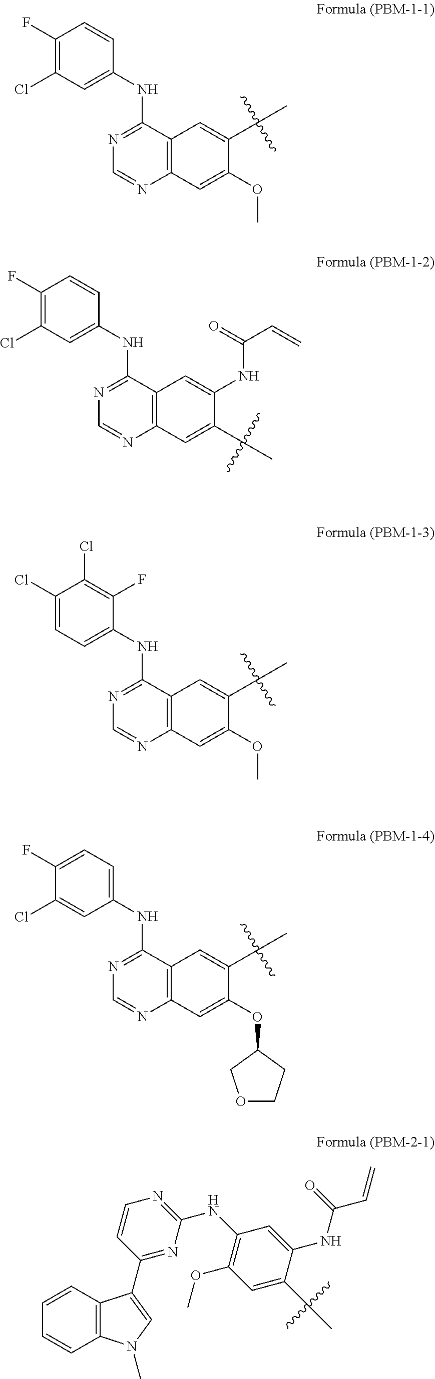 [SMILES: *c1cc2c(Nc3ccc(F)c(Cl)c3)ncnc2cc1OC ; *c1cc2ncnc(Nc3ccc(F)c(Cl)c3)c2cc1NC(=O)C=C ; *c1cc2c(Nc3ccc(Cl)c(Cl)c3F)ncnc2cc1OC ; *c1cc2c(Nc3ccc(F)c(Cl)c3)ncnc2cc1O[C@H]1CCOC1 ; *c1cc(OC)c(Nc2nccc(-c3cn(C)c4ccccc34)n2)cc1NC(=O)C=C] 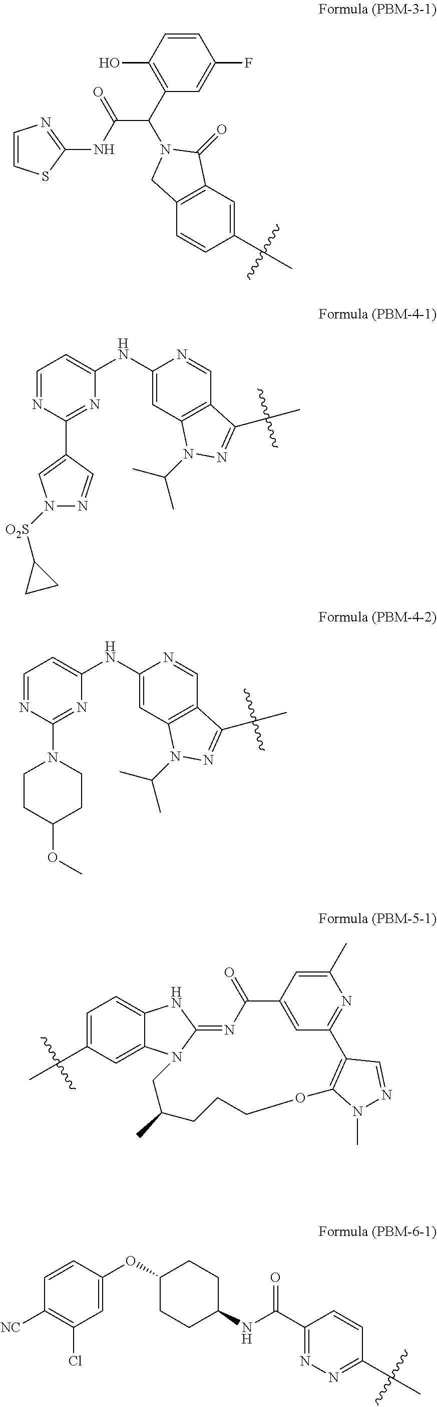 [SMILES: *c1ccc2c(c1)C(=O)N(C(C(=O)Nc1nccs1)c1cc(F)ccc1O)C2 ; *c1nn(C(C)C)c2cc(Nc3ccnc(-c4cnn(S(=O)(=O)C5CC5)c4)n3)ncc12 ; *c1nn(C(C)C)c2cc(Nc3ccnc(N4CCC(OC)CC4)n3)ncc12 ; *c1ccc2c(c1)N1C[C@H](C)CCCOc3c(cnn3C)-c3cc(cc(C)n3)C(=O)/N=C/1N2 ; *c1ccc(C(=O)N[C@H]2CC[C@H](Oc3ccc(C#N)c(Cl)c3)CC2)nn1] 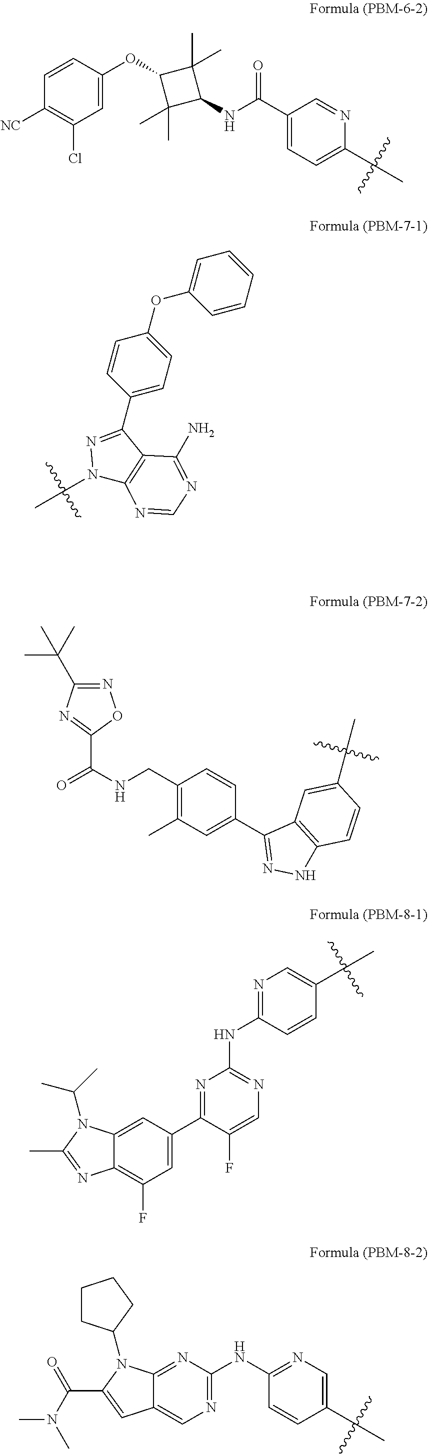 [SMILES: *c1ccc(C(=O)N[C@H]2C(C)(C)[C@H](Oc3ccc(C#N)c(Cl)c3)C2(C)C)cn1 ; *n1nc(-c2ccc(Oc3ccccc3)cc2)c2c(N)ncnc21 ; *c1ccc2[nH]nc(-c3ccc(CNC(=O)c4nc(C(C)(C)C)no4)c(C)c3)c2c1 ; *c1ccc(Nc2ncc(F)c(-c3cc(F)c4nc(C)n(C(C)C)c4c3)n2)nc1 ; *c1ccc(Nc2ncc3cc(C(=O)N(C)C)n(C4CCCC4)c3n2)nc1] 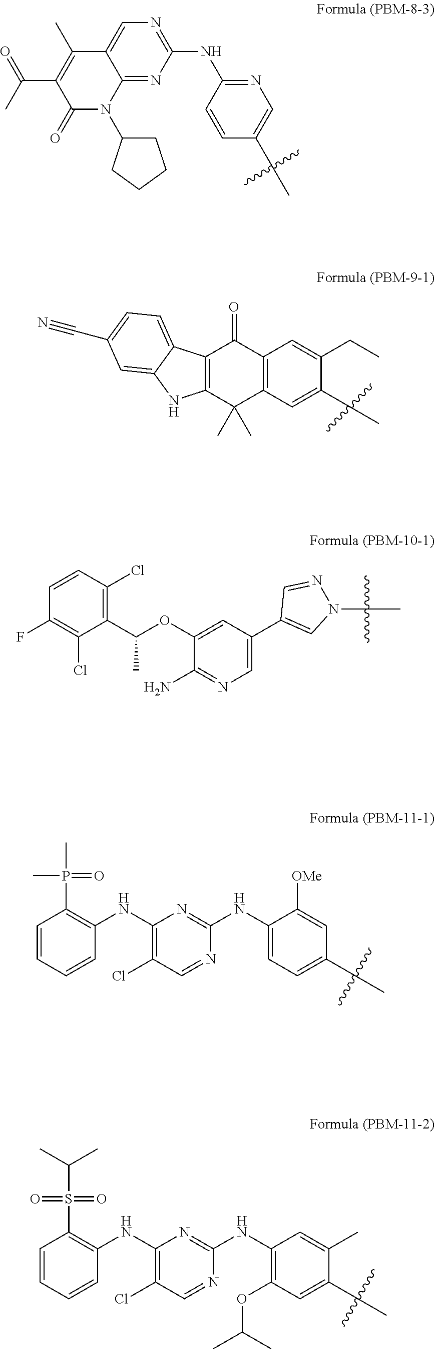 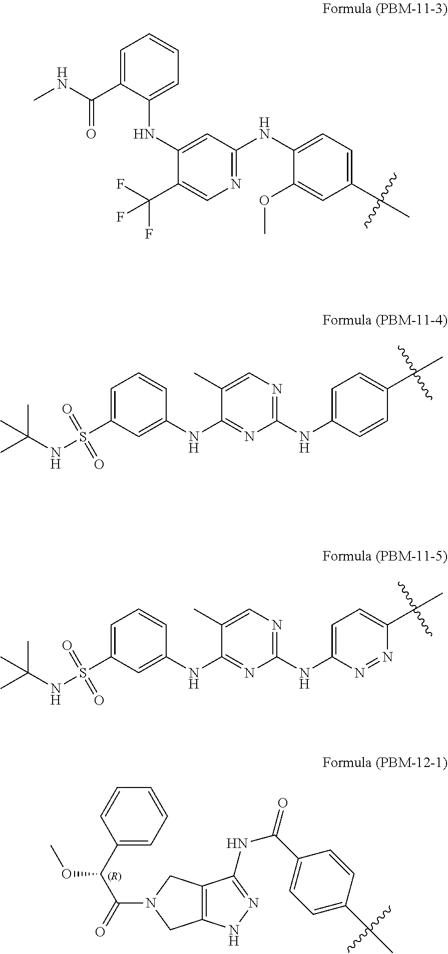 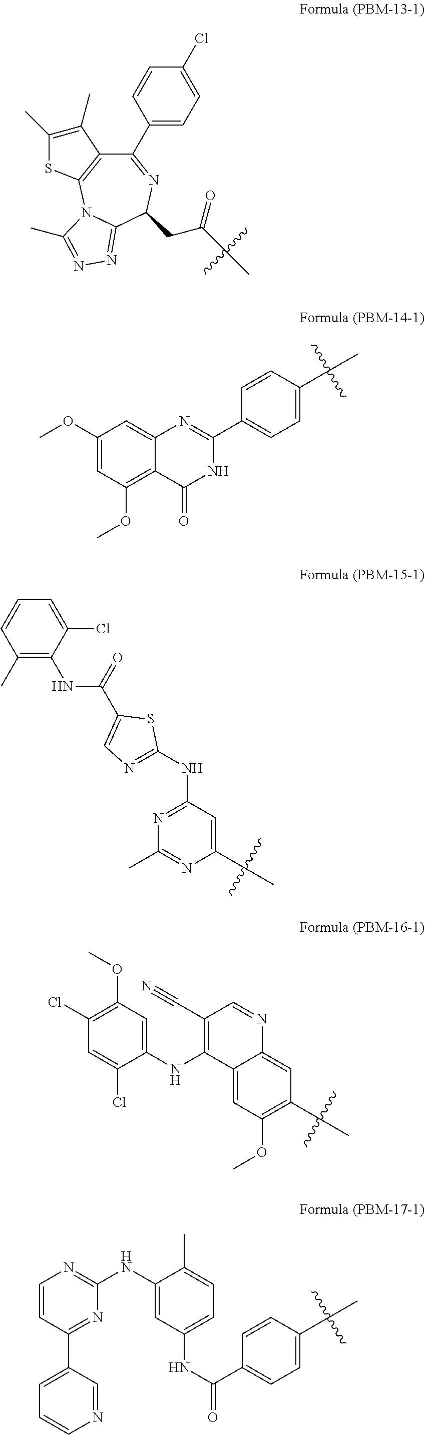 [SMILES: *C(=O)C[C@@H]1N=C(c2ccc(Cl)cc2)c2c(sc(C)c2C)-n2c(C)nnc21 ; *c1ccc(-c2nc3cc(OC)cc(OC)c3c(=O)[nH]2)cc1 ; *c1cc(Nc2ncc(C(=O)Nc3c(C)cccc3Cl)s2)nc(C)n1 ; *c1cc2ncc(C#N)c(Nc3cc(OC)c(Cl)cc3Cl)c2cc1OC ; *c1ccc(C(=O)Nc2ccc(C)c(Nc3nccc(-c4cccnc4)n3)c2)cc1] 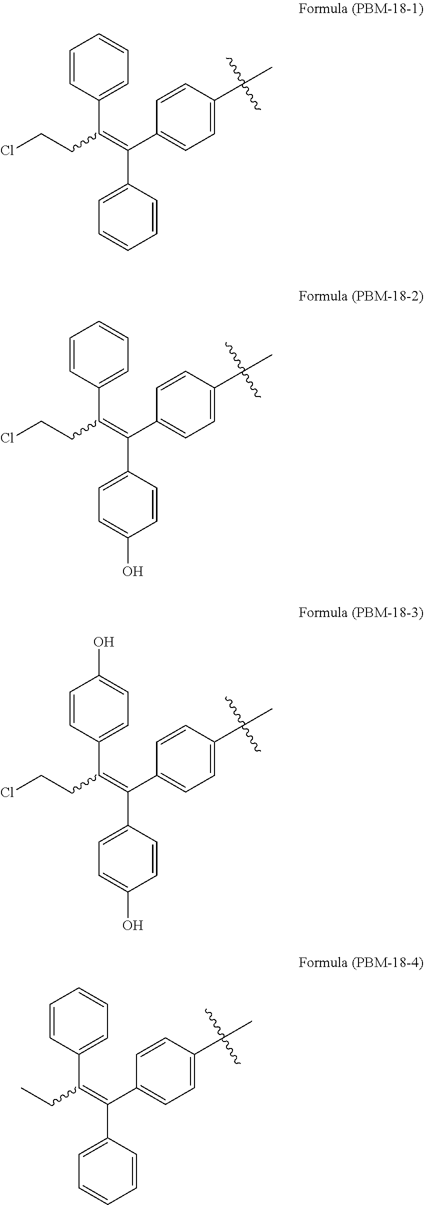 [SMILES: *c1ccc(C(=C(CCCl)c2ccccc2)c2ccccc2)cc1 ; *c1ccc(C(=C(CCCl)c2ccccc2)c2ccc(O)cc2)cc1 ; *c1ccc(C(=C(CCCl)c2ccc(O)cc2)c2ccc(O)cc2)cc1 ; *c1ccc(C(=C(CC)c2ccccc2)c2ccccc2)cc1] 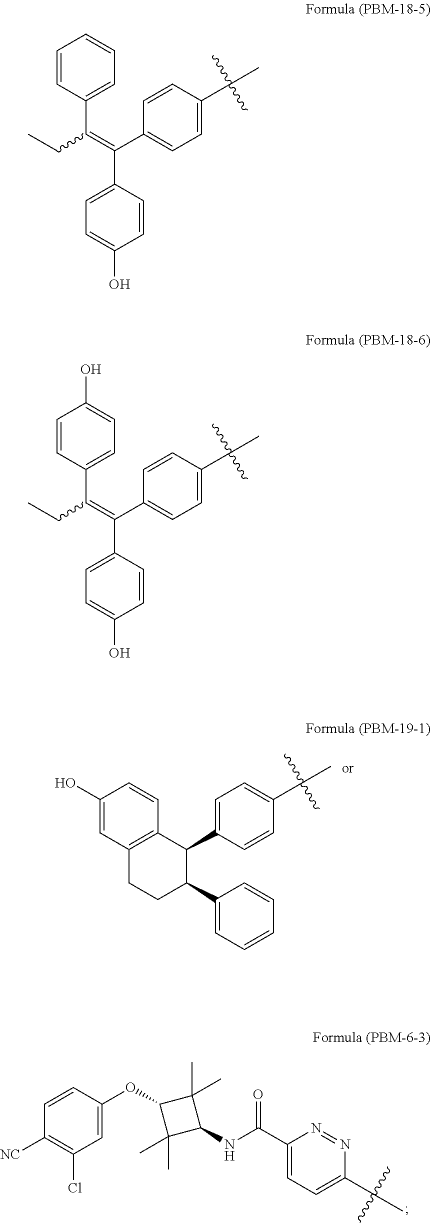 [SMILES: *c1ccc(C(=C(CC)c2ccccc2)c2ccc(O)cc2)cc1 ; *c1ccc(C(=C(CC)c2ccc(O)cc2)c2ccc(O)cc2)cc1 ; *c1ccc([C@@H]2c3ccc(O)cc3CC[C@@H]2c2ccccc2)cc1 ; *c1ccc(C(=O)N[C@H]2C(C)(C)[C@H](Oc3ccc(C#N)c(Cl)c3)C2(C)C)nn1]
and/or
(ii) the W¹ represents the following groups: 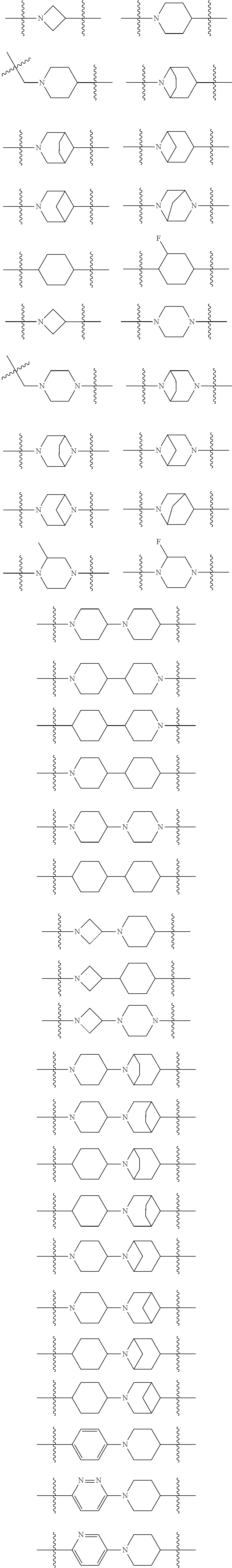 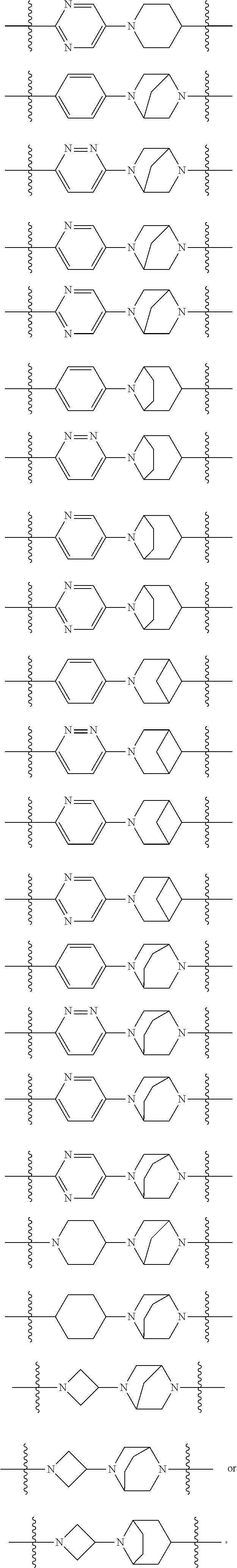
wherein the groups are optionally substituted with one or more substituents selected from the group consisting of deuterium, halogen, cyano, amino, C₁₋₆ alkyl, halogenated C₁₋₆ alkyl, deuterated C₁₋₆ alkyl, C₁₋₆ alkoxy, halogenated C₁₋₆ alkoxy, deuterated C₁₋₆ alkoxy, or any combination thereof;
or
(iii) the R¹—W¹ represents the following groups: 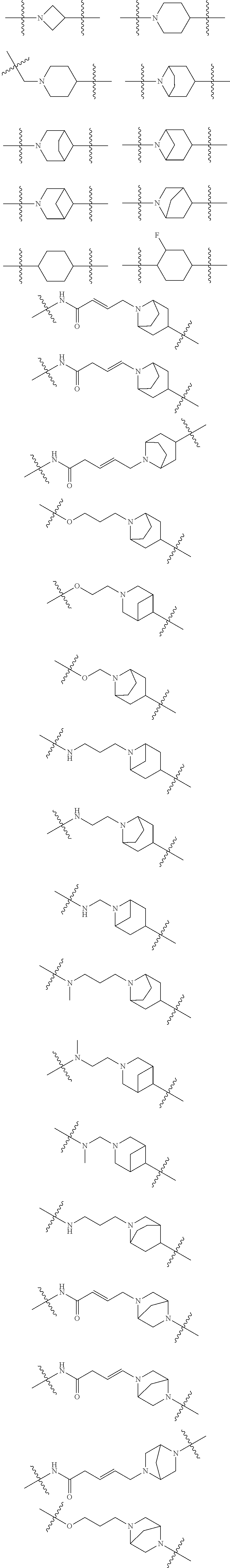 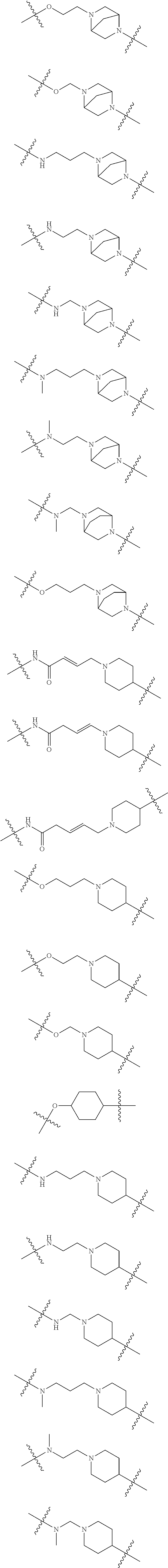 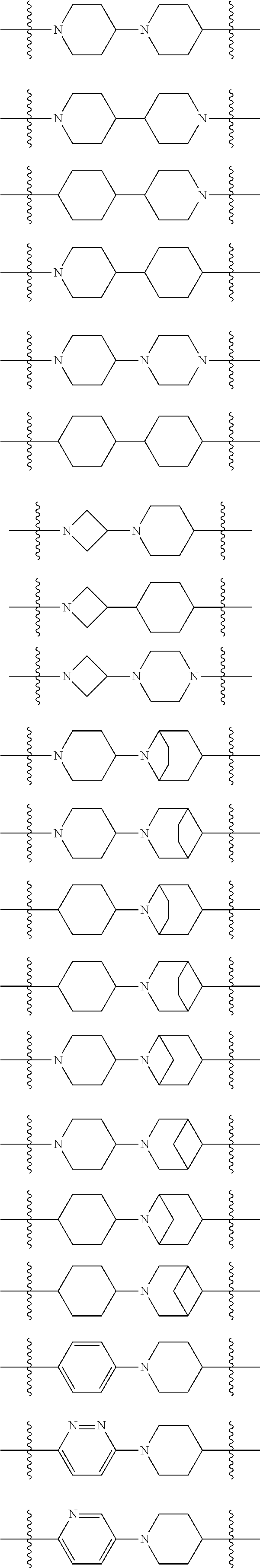 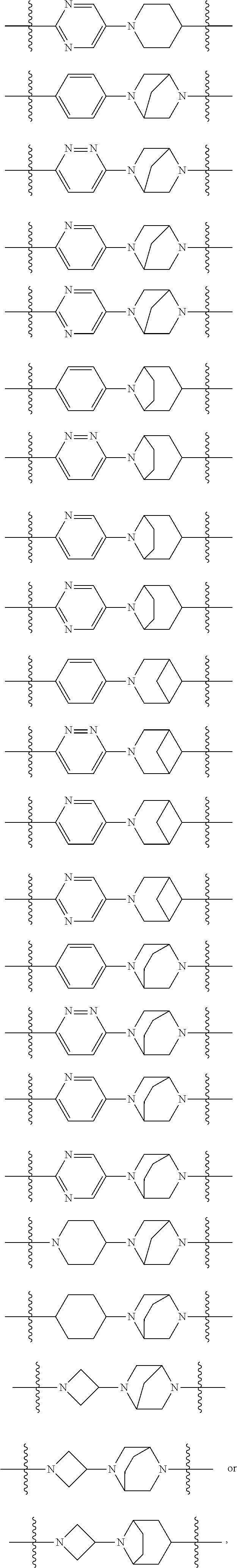
wherein the groups are optionally substituted with one or more substituents selected from the group consisting of deuterium, halogen, cyano, amino, C₁₋₆ alkyl, halogenated C₁₋₆ alkyl, deuterated C₁₋₆ alkyl, C₁₋₆ alkoxy, halogenated C₁₋₆ alkoxy, deuterated C₁₋₆ alkoxy, or any combination thereof;
or
(iv) the R_{c} represents the following groups: 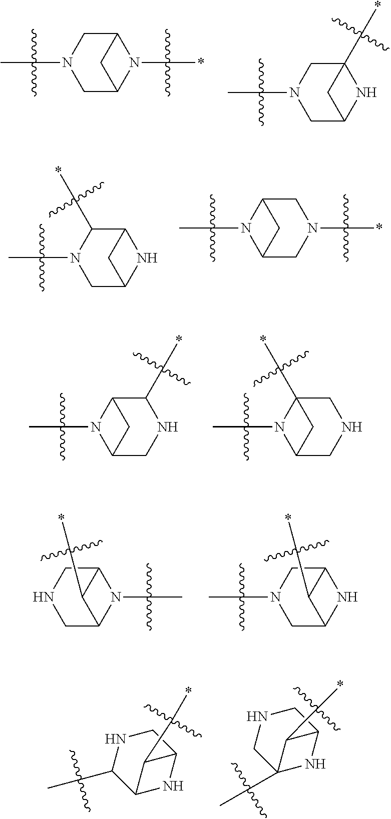 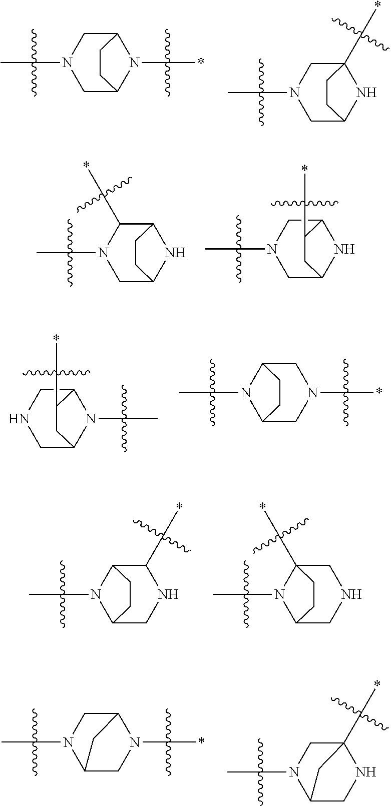 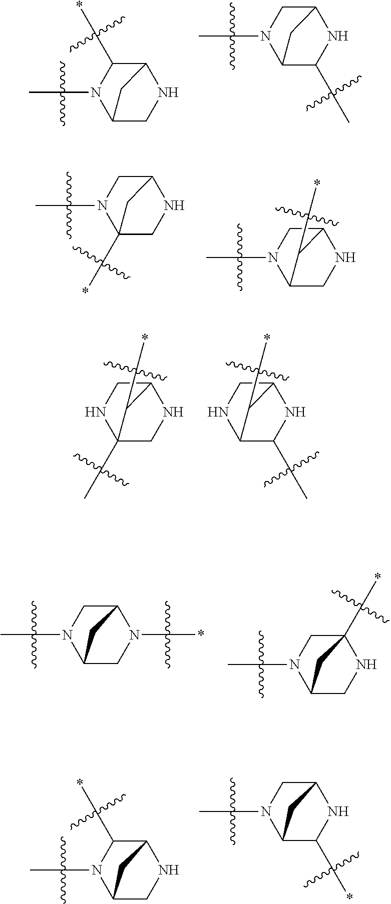 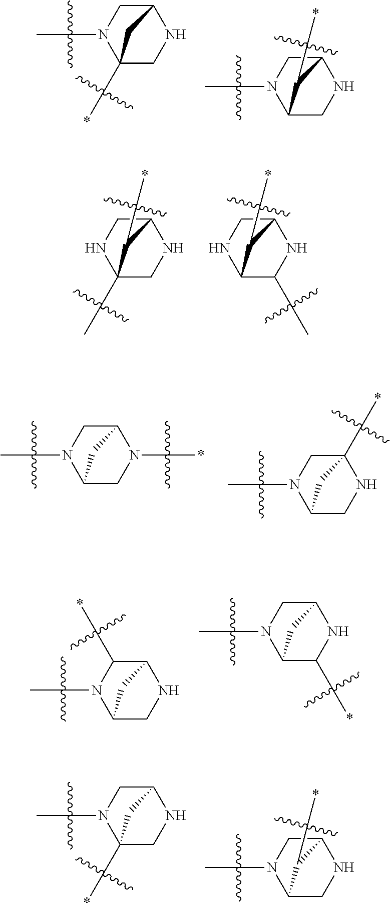 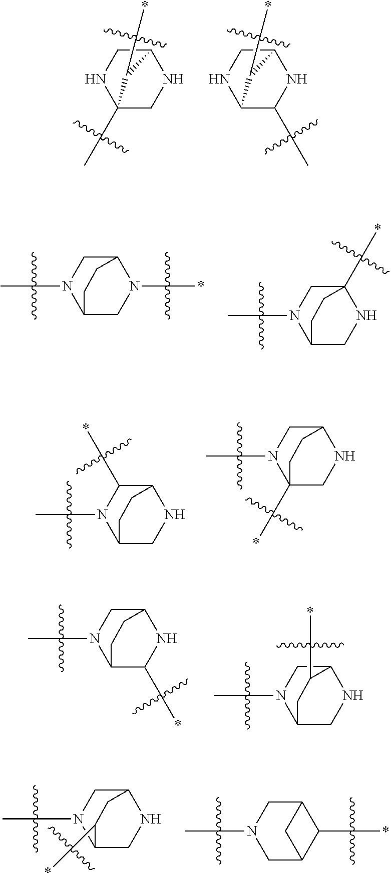 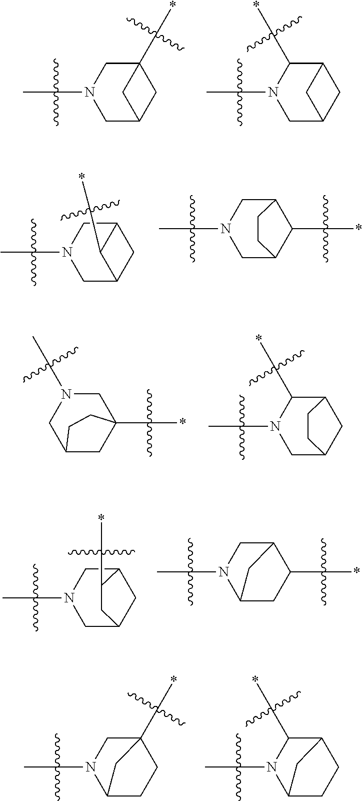 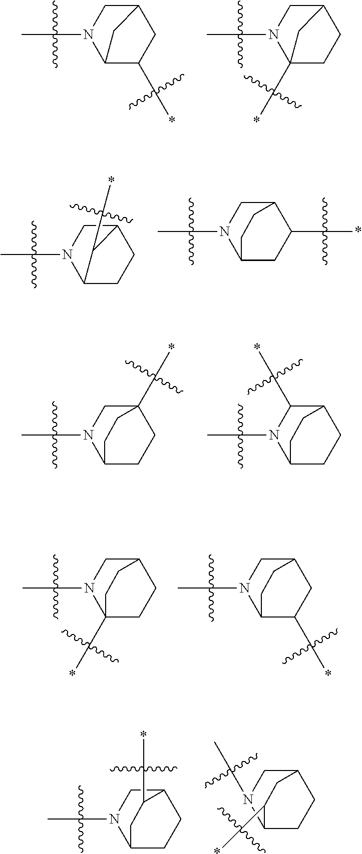 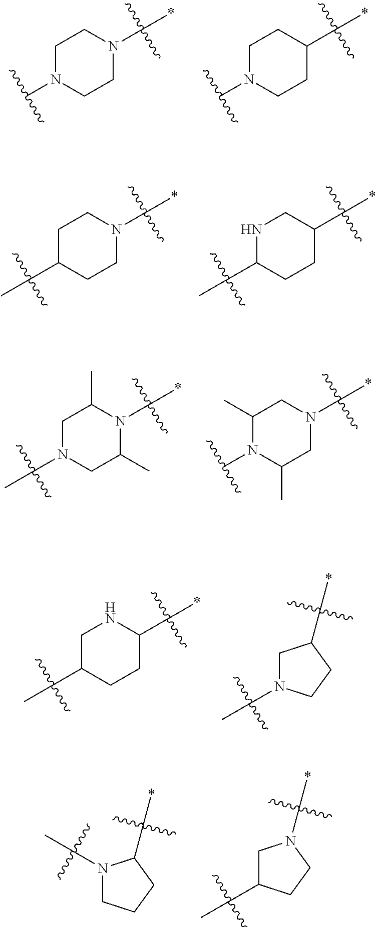 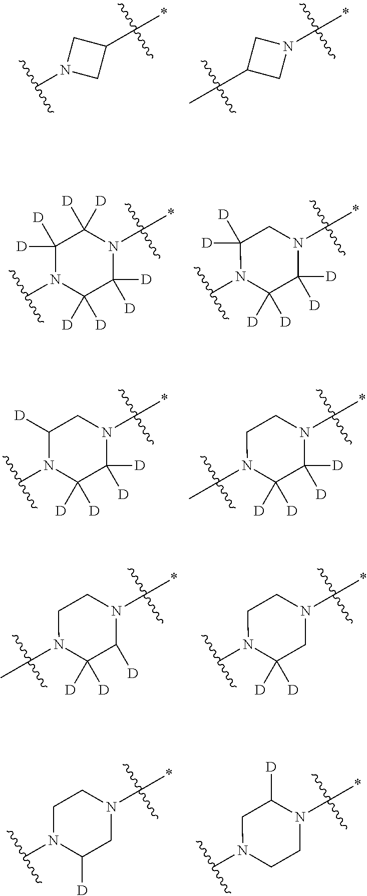 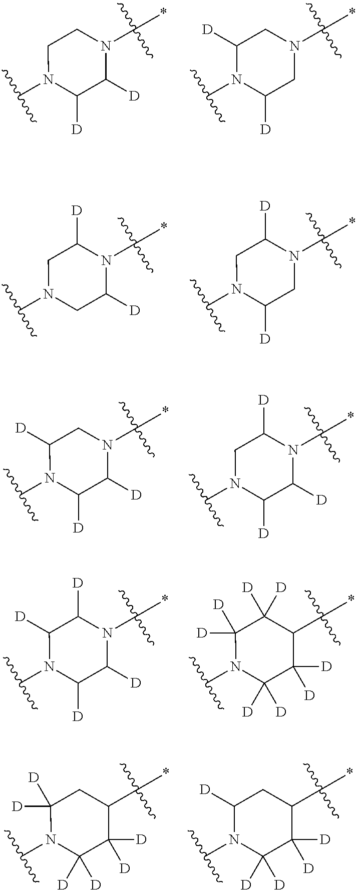 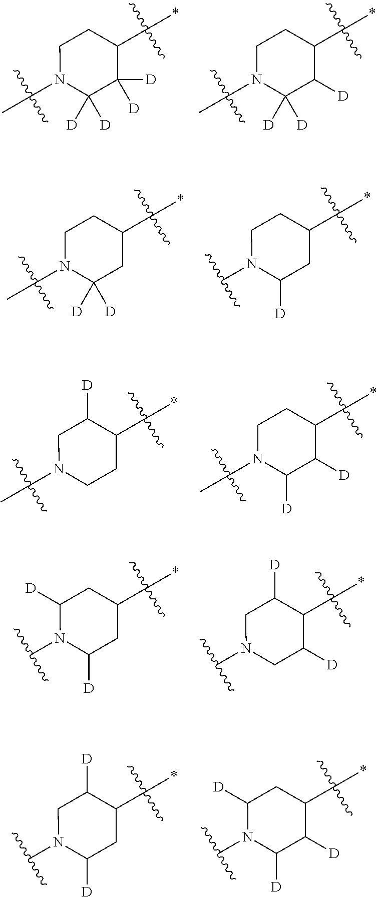 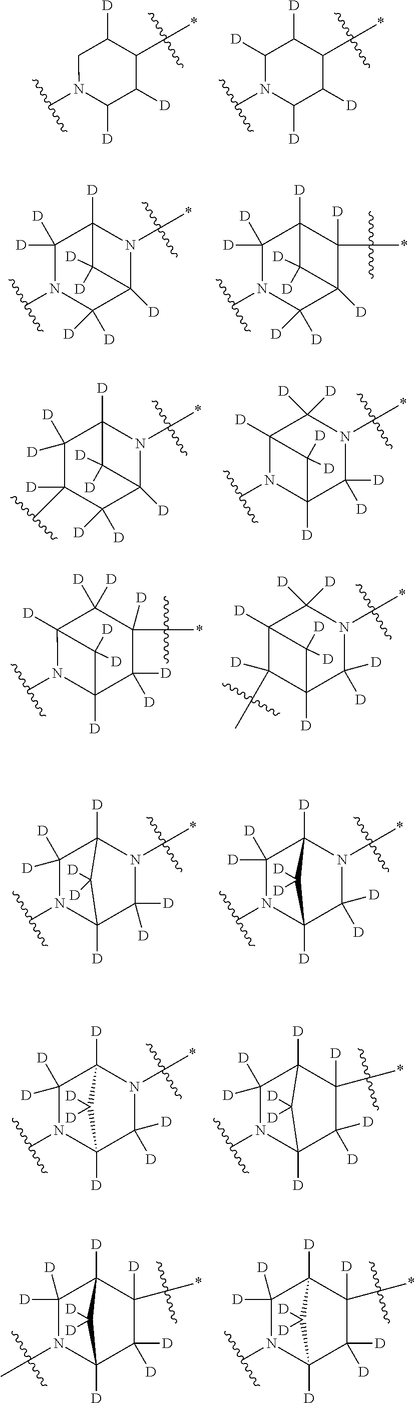 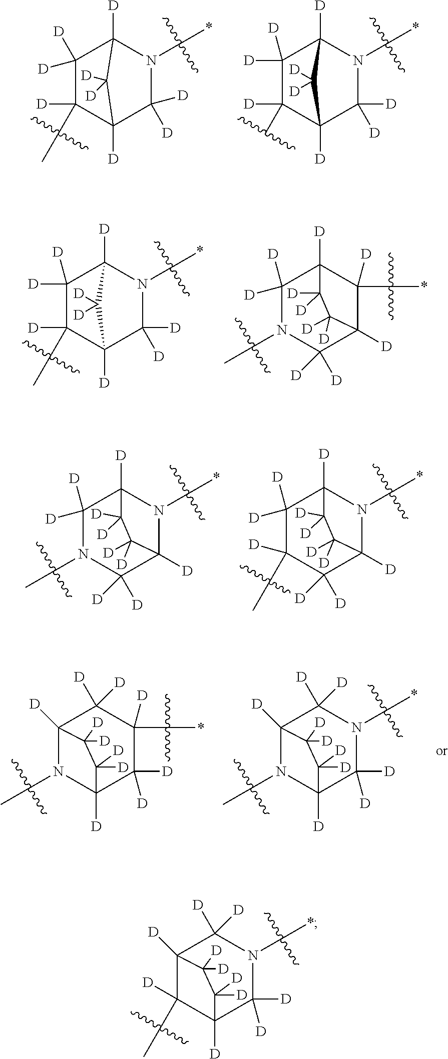
wherein symbol * indicates the point of attachment to U₁;
and/or
(v) wherein LIN represents the structure of the following formula:
#-(C(R^{a1})(R^{a2}))ₙ₁—(O(C(R^{a3})(R^{a4}))ₙ₂)ₘ—;
#-(C(R^{a1})(R^{a2}))ₙ₁—(O(C(R^{a3})(R^{a4}))ₙ₂)ₘ₁—(O(C(R^{a5})(R^{a6}))ₙ₃)ₘ₂—;
#-(C(R^{a1})(R^{a2}))ₙ₁—(O(C(R^{a3})(R^{a4}))ₙ₂)ₘ₁—(O(C(R^{a5})(R^{a6}))ₙ₃)ₘ₂—(O(C(R^{a7})(R^{a8}))ₙ₄)ₘ₃—;
#-(C(R^{a1})(R^{a2}))ₙ₁—(N(R_{g})(C(R^{a3})(R^{a4}))ₙ₂)ₘ₁—;
#-(C(R^{a1})(R^{a2}))ₙ₁—(N(R_{g})(C(R^{a3})(R^{a4}))ₙ₂)ₘ₁—(N(R_{g})(C(R^{a5})(R^{a6}))ₙ₃)ₘ₂—;
#-(C(R^{a1})(R^{a2}))ₙ₁—(N(R_{g})(C(R^{a3})(R^{a4}))ₙ₂)ₘ₁—(N(R_{g})(C(R^{a5})(R^{a6}))ₙ₃)ₘ₂—(N(R_{g})(C(R^{a7})(R^{a8}))ₙ₄)ₘ₃—;
#-(C(R^{a1})(R^{a2}))ₙ₁—(C(O)N(R_{g})—(C(R^{a3})(R^{a4}))ₙ₂)ₘ₁—;
#-(C(R^{a1})(R^{a2}))ₙ₁—(C(O)N(R_{g})—(C(R^{a3})(R^{a4}))ₙ₂)ₘ₁—(C(O)N(R_{g})—(C(R^{a5})(R^{a6}))ₙ₃)ₘ₂—;
#-(C(R^{a1})(R^{a2}))ₙ₁—(C(O)N(R_{g})—(C(R^{a3})(R^{a4}))ₙ₂)ₘ₁—(C(O)N(R_{g})—(C(R^{a5})(R^{a6}))ₙ₃)ₘ₂—(C(O)N(R_{g})—(C(R^{a7})(R^{a8}))ₙ₄)ₘ₃—;
#-(C(R^{a1})(R^{a2}))ₙ₁—(C(O)N(R_{g})—(C(R^{a3})(R^{a4}))ₙ₂)ₘ₁—(O—(C(R^{a5})(R^{a6}))ₙ₃)ₘ₂—;
#-(C(R^{a1})(R^{a2}))ₙ₁—C(O)N(R_{g})—(C(R^{a3})(R^{a4}))ₙ₂—(O(C(R^{a5})(R^{a6}))ₙ₃)ₘ₁—;
#-(C(R^{a1})(R^{a2}))ₙ₁—(N(R_{g})C(O)—(C(R^{a3})(R^{a4}))ₙ₂)ₘ₁—;
#-(C(R^{a1})(R^{a2}))ₙ₁—(N(R_{g})C(O)—(C(R^{a3})(R^{a4}))ₙ₂)ₘ₁—(O—(C(R^{a5})(R^{a6}))ₙ₃)ₘ₂—;
#-(C(R^{a1})(R^{a2}))ₙ₁—(N(R_{g})C(O)—(C(R^{a3})(R^{a4}))ₙ₂)ₘ₁—(O—(C(R^{a5})(R^{a6}))ₙ₃)ₘ₂—(O—(C(R_{g})(R^{a8}))ₙ₄)ₘ₃—;
#-(C(R^{a1})(R^{a2}))ₙ₁—N(R_{g})C(O)—(C(R^{a3})(R^{a4}))ₙ₂—(O(C(R^{a5})(R^{a6}))ₙ₃)ₘ₁—;
#-(C(R^{a1})(R^{a2}))ₙ₁—N(R_{g})C(O)N(R_{g})—(C(R^{a3})(R^{a4}))ₙ₂—;
#-(C(R^{a1})(R^{a2}))ₙ₁—C(O)—(C(R^{a3})(R^{a4}))ₙ₂—;
#-(C(R^{a1})(R^{a2}))ₙ₁-(arylene-(C(R^{a3})(R^{a4}))ₙ₂)ₘ₁—;
#-(C(R^{a1})(R^{a2}))ₙ₁-(arylene-(C(R^{a3})(R^{a4}))ₙ₂)ₘ₁-arylene-(C(R^{a5})(R^{a6}))ₙ₃—;
#-(C(R^{a1})(R^{a2}))ₙ₁-(heterocyclylene-(C(R^{a3})(R^{a4}))₂)ₘ₁—;
#-(C(R^{a1})(R^{a2}))ₙ₁-(heterocyclylene-(C(R^{a3})(R^{a4}))ₙ₂)ₘ₁-(heterocyclylene-(C(R^{a5})(R^{a6}))ₙ₃)ₘ₂—;
#-(C(R^{a1})(R^{a2}))ₙ₁-(heteroarylene-(C(R^{a3})(R^{a4}))ₙ₂)ₘ₁—;
#-(C(R^{a1})(R^{a2}))ₙ₁-(heteroarylene-(C(R^{a3})(R^{a4}))ₙ₂)ₘ₁-(heteroarylene-(C(R^{a5})(R^{a6}))ₙ₃)ₘ₂—;
#-(C(R^{a1})(R^{a2}))ₙ₁-cycloalkylene-(C(R^{a3})(R^{a4}))ₙ₂)ₘ₁—; or
#-(C(R^{a1})(R^{a2}))ₙ₁-cycloalkylene-(C(R^{a3})(R^{a4}))ₙ₂)ₘ₁-(cycloalkylene-(C(R^{a5})(R^{a6}))ₙ₃)ₘ₂—;
wherein each R_{g} independently represent H or C₁₋₆ alkyl;
the cycloalkylene, arylene, heterocyclylene, and heteroarylene are each independently optionally substituted with a substituent selected from the group consisting of deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano, or any combination thereof;
R^{a1}, R^{a2}, R^{a3}, R^{a4}, R^{a5}, R^{a6}, R^{a7} and R^{a8} each independently represent H, deuterium, hydroxy, amino, mercapto, nitro, halogen, cyano, optionally deuterated C₁₋₆ alkyl, halogenated C₁₋₆ alkyl, optionally deuterated C₃₋₆ cycloalkyl, optionally deuterated C₁₋₆ alkoxy, optionally deuterated C₁₋₆ alkyl-NH—, NH₂—C₁₋₆ alkylene, optionally deuterated C₁₋₆ alkyl-NHC(O)—, optionally deuterated C₁₋₆ alkyl-C(O)NH—, or any combination thereof;
symbol # indicates the point of attachment to U₂; and
n1, n2, n3, n4, m1, m2, and m3 each independently represent an integer of 1, 2, 3, 4, 5, 6, 7, 8, 9, 10, 11, 12, 13, 14, or 15.

24-29. (canceled)

30. The compound of Formula (III) or salts, enantiomers, stereoisomers, solvates, isotopically enriched analogs, prodrugs, or polymorphs thereof as claimed in claim 20, wherein (i) the ULM represents the structures of Formula (ULM-11), (ULM-12), (ULM-13), (ULM-14), (ULM-15), (ULM-16), (ULM-17), (ULM-18), (ULM-19), (ULM-20), (ULM-21) or (ULM-22): 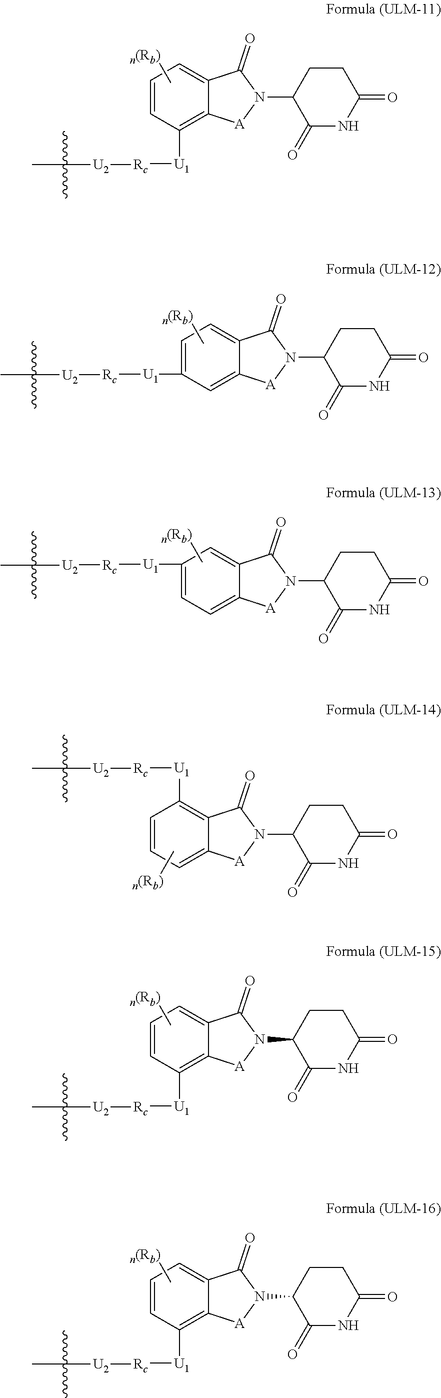 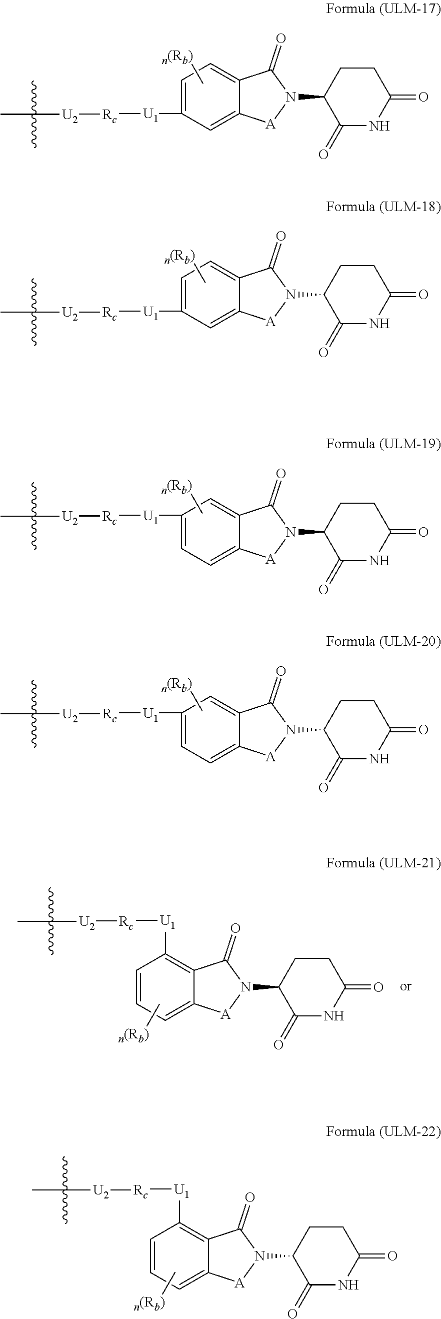
wherein A, (R_{b})ₙ, R_{b}, R_{c}, U₁, and U₂ are as defined in claim 20;
or
(ii) LIN represents the following groups:
#-CH₂—, #-(CH₂)₂—, #-(CH₂)₃—, #-(CH₂)₄—, #-(CH₂)₅—, #-(CH₂)₆—, #-(CH₂)₇—, #- (CH₂)₈—, #-(CH₂)₉—, #-(CH₂)₁₀—, #-(CH₂)₁₁—, #-(CH₂)₁₂—, #-(CH₂)₁₃—, #-(CH₂)₁₄—, #-(CH₂)₁₅—, #-(CH₂)₁₆—, #-(CH₂)₁₇—, #-(CH₂)₁₈—, #-(CH₂)₁₉—, #-(CH₂)₂₀—, #-(CH₂)₂₁—, #-(CH₂)₂₂—, #-(CH₂)₂₅—, or #-(CH₂)₃₀—; #-CH₂—O—CH₂—, #-CH₂—O—(CH₂)₂—, #-(CH₂)₁—O—(CH₂)₃—, #-(CH₂)₁—O—(CH₂)₄—, #-(CH₂)₁—O—(CH₂)₅—, #-(CH₂)₁—O—(CH₂)₆—, #-(CH₂)₁—O—(CH₂)₇—, #-(CH₂)₁—O—(CH₂)₈—, #-(CH₂)₁—O—(CH₂)₉—, #-(CH₂)₁—O—(CH₂)₁₀—, #-(CH₂)₂—O—(CH₂)₁—, #-(CH₂)₂—O—(CH₂)₂—, #-(CH₂)₂—O—(CH₂)₃—, #-(CH₂)₂—O—(CH₂)₄—, #-(CH₂)₂—O—(CH₂)₅—, #-(CH₂)₂—O—(CH₂)₆—, #-(CH₂)₂—O—(CH₂)₇—, #-(CH₂)₂—O—(CH₂)₈—, #-(CH₂)₂—O—(CH₂)₉—, #-(CH₂)₂—O—(CH₂)₁₀—, #-(CH₂)₂—O—(CH₂)₁₁—, #-(CH₂)₂—O—(CH₂)₁₂—, #-(CH₂)₃—O—(CH₂)₁—, #-(CH₂)₃—O—(CH₂)₂—, #-(CH₂)₃—O—(CH₂)₃—, #-(CH₂)₃—O—(CH₂)₄—, #-(CH₂)₃—O—(CH₂)₅—, #-(CH₂)₃—O—(CH₂)₆—, #-(CH₂)₃—O—(CH₂)₇—, #-(CH₂)₄—O—(CH₂)₁—, #-(CH₂)₄—O—(CH₂)₂—, #-(CH₂)₄—O—(CH₂)₃—, #-(CH₂)₄—O—(CH₂)₄—, #-(CH₂)₄—O—(CH₂)₅—, #-(CH₂)₄—O—(CH₂)₆—, #-(CH₂)₅—O—(CH₂)₁—, #-(CH₂)₅—O—(CH₂)₂—, #-(CH₂)₅—O—(CH₂)₃—, #-(CH₂)₅—O—(CH₂)₄—, #-(CH₂)₅—O—(CH₂)₅—, #-(CH₂)₆—O—(CH₂)₁—, #-(CH₂)₆—O—(CH₂)₂—, #-(CH₂)₆—O—(CH₂)₃—, #-(CH₂)₆—O—(CH₂)₄—, #-(CH₂)₇—O—(CH₂)₁—, #-(CH₂)₇—O—(CH₂)₂—, #-(CH₂)₇—O—(CH₂)₃—, #-(CH₂)₈—O—(CH₂)₁—, #-(CH₂)₈—O—(CH₂)₂—, #-CH(CH₃)—O—(CH₂)₁—, #-CH(CH₃)—O—(CH₂)₂—, #-CH(CH₃)—O—(CH₂)₃—, #-CH(CH₃)—O—(CH₂)₄—, #-CH(CH₃)—O—(CH₂)₅—, #-CH(CH₃)—O—(CH₂)₆—, #-CH(CH₃)—O—(CH₂)₇—, #-CH(CH₃)—O—(CH₂)₈—, #-CH(CH₃)—O—(CH₂)₉—, #-CH(CH₃)—O—(CH₂)₁₀—, #-CH₂—(O(CH₂)₂)₂—, #-CH₂—(O(CH₂)₂)₃—, #-CH₂—(O(CH₂)₂)₄—, #-CH₂—(O(CH₂)₂)₅—, #-CH₂—(O(CH₂)₂)₆—, #-CH₂—(O(CH₂)₂)₇—, #-CH₂—(O(CH₂)₂)₈—, #-CH₂—(O(CH₂)₂)₉—, #-CH₂—(O(CH₂)₂)₁₀—, #-CH₂—(O(CH₂)₂)₁—OCH₂—, #-CH₂—(O(CH₂)₂)₂—OCH₂—, #-CH₂—(O(CH₂)₂)₃—OCH₂—, #-CH₂—(O(CH₂)₂)₄—OCH₂—, #-CH₂—(O(CH₂)₂)₅—OCH₂—, #-CH₂—(O(CH₂)₂)₆—OCH₂—, #-CH₂—(O(CH₂)₂)₇—OCH₂—, #-CH₂—(O(CH₂)₂)₈—OCH₂—, #-CH₂—(O(CH₂)₂)₉—OCH₂—, #-CH₂—(O(CH₂)₂)₁₀—OCH₂—, #-(CH₂)₂—(O(CH₂)₂)₂—, #-(CH₂)₂—(O(CH₂)₂)₃—, #-(CH₂)₂—(O(CH₂)₂)₄—, #-(CH₂)₂—(O(CH₂)₂)₅—, #-(CH₂)₂—(O(CH₂)₂)₆—, #-(CH₂)₂—(O(CH₂)₂)₇—, #-(CH₂)₂—(O(CH₂)₂)₈—, #-(CH₂)₂—(O(CH₂)₂)₉—, #-(CH₂)₂—(O(CH₂)₂)₁₀—, #-(CH₂)₃—(O(CH₂)₂)₂—, #-(CH₂)₃—(O(CH₂)₂)₃—, #-(CH₂)₃—(O(CH₂)₂)₄—, #-(CH₂)₃—(O(CH₂)₂)₅—, #-(CH₂)₃—(O(CH₂)₂)₆—, #-(CH₂)₃—(O(CH₂)₂)₇—, #-(CH₂)₃—(O(CH₂)₂)₈—, #-(CH₂)₃—(O(CH₂)₂)₉—, #-(CH₂)₃—(O(CH₂)₂)₁₀—, #-(CH₂)₄—(O(CH₂)₂)₂—, #-(CH₂)₄—(O(CH₂)₂)₃—, #-(CH₂)₄—(O(CH₂)₂)₄—, #-(CH₂)₄—(O(CH₂)₂)₅—, #-(CH₂)₄—(O(CH₂)₂)₆—, #-(CH₂)₄—(O(CH₂)₂)₇—, #-(CH₂)₄—(O(CH₂)₂)₈—, #-(CH₂)₄—(O(CH₂)₂)₉—, #-(CH₂)₄—(O(CH₂)₂)₁₀—, #-CH₂—(O(CH₂)₃)₂—, #-CH₂—(O(CH₂)₃)₃—, #-CH₂—(O(CH₂)₃)₄—, #-CH₂—(O(CH₂)₃)₅—, #-CH₂—(O(CH₂)₃)₆—, #-CH₂—(O(CH₂)₃)₇—, #-CH₂—(O(CH₂)₃)₈—, #-CH₂—(O(CH₂)₃)₉—, #- CH₂—(O(CH₂)₃)₁₀—, #-(CH₂)₂—(O(CH₂)₃)₂—, #-(CH₂)₂—(O(CH₂)₃)₃—, #-(CH₂)₂—(O(CH₂)₃)₄—, #-(CH₂)₂—(O(CH₂)₃)₅—, #-(CH₂)₂—(O(CH₂)₃)₆—, #-(CH₂)₂—(O(CH₂)₃)₇—, #-(CH₂)₂—(O(CH₂)₃)₈—, #-(CH₂)₂—(O(CH₂)₃)₉—, #-(CH₂)₂—(O(CH₂)₃)₁₀—, #-(CH₂)₃—(O(CH₂)₃)₂—, #-(CH₂)₃—(O(CH₂)₃)₃—, #-(CH₂)₃—(O(CH₂)₃)₄—, #-(CH₂)₃—(O(CH₂)₃)₅—, #-(CH₂)₃—(O(CH₂)₃)₆—, #-(CH₂)₃—(O(CH₂)₃)₇—, #-(CH₂)₃—(O(CH₂)₃)₈—, #-(CH₂)₃—(O(CH₂)₃)₉—, #-(CH₂)₃—(O(CH₂)₃)₁₀—, #-CH₂—O—(CH₂)₂—O—(CH₂)₃—, #-CH₂—(O(CH₂)₂)₂—(O(CH₂)₃)₂—, #-CH₂—(O(CH₂)₂)₃—(O(CH₂)₃)₃—, #-CH₂—(O(CH₂)₂)₄—(O(CH₂)₃)₄—, #-CH₂—(O(CH₂)₂)₅—(O(CH₂)₃)₅—, #-CH₂—(O(CH₂)₂)₆—(O(CH₂)₃)₆—, #-(CH₂)₂—O—(CH₂)₂—O—(CH₂)₃—, #-(CH₂)₂—(O(CH₂)₂)₂—(O(CH₂)₃)₂—, #-(CH₂)₂—(O(CH₂)₂)₃—(O(CH₂)₃)₃—, #-(CH₂)₂—(O(CH₂)₂)₄—(O(CH₂)₃)₄—, #-(CH₂)₂—(O(CH₂)₂)₅—(O(CH₂)₃)₅—, #-(CH₂)₂—(O(CH₂)₂)₆—(O(CH₂)₃)₆—, #-(CH₂)₃—O—(CH₂)₂—O—(CH₂)₃—, #-(CH₂)₃—(O(CH₂)₂)₂—(O(CH₂)₃)₂—, #-(CH₂)₃—(O(CH₂)₂)₃—(O(CH₂)₃)₃—, #-(CH₂)₃—(O(CH₂)₂)₄—(O(CH₂)₃)₄—, #-(CH₂)₃—(O(CH₂)₂)₅—(O(CH₂)₃)₅—, #-(CH₂)₃—(O(CH₂)₂)₆—(O(CH₂)₃)₆—, #-CH₂—O—(CH₂)₃—O—(CH₂)₂—, #-CH₂—(O(CH₂)₃)₂—(O(CH₂)₂)₂—, #-CH₂—(O(CH₂)₃)₃—(O(CH₂)₂)₃—, #-CH₂—(O(CH₂)₃)₄—(O(CH₂)₂)₄—, #-CH₂—(O(CH₂)₃)₅—(O(CH₂)₂)₅—, #-CH₂—(O(CH₂)₃)₆—(O(CH₂)₂)₆—, #-(CH₂)₂—O—(CH₂)₃—O—(CH₂)₂—, #-(CH₂)₂—(O(CH₂)₃)₂—(O(CH₂)₂)₂—, #-(CH₂)₂—(O(CH₂)₃)₃—(O(CH₂)₂)₃—, #-(CH₂)₂—(O(CH₂)₃)₄—(O(CH₂)₂)₄—, #-(CH₂)₂—(O(CH₂)₃)₅—(O(CH₂)₂)₅—, #-(CH₂)₂—(O(CH₂)₃)₆—(O(CH₂)₂)₆—, #-(CH₂)₃—O—(CH₂)₃—O—(CH₂)₂—, #-(CH₂)₃—(O(CH₂)₃)₂—(O(CH₂)₂)₂—, #-(CH₂)₃—(O(CH₂)₃)₃—(O(CH₂)₂)₃—, #-(CH₂)₃—(O(CH₂)₃)₄—(O(CH₂)₂)₄—, #-(CH₂)₃—(O(CH₂)₃)₅—(O(CH₂)₂)₅—, #-(CH₂)₃—(O(CH₂)₃)₆—(O(CH₂)₂)₆—, #-CH₂—O—(CH₂)₂O—CH₂—, #-(CH₂)₂—O—(CH₂)₂O—CH₂—, #-(CH₂)₂—(O(CH₂)₂)₂—O—(CH₂)₃—, #-(CH₂)₂—(O(CH₂)₂)₃—O—(CH₂)₃—, #-(CH₂)₂—(O(CH₂)₂)₄—O—(CH₂)₃—, #-(CH₂)₅—(O(CH₂)₂)₂—O—(CH₂)₅—, #-(CH₂)₅—(O(CH₂)₂)₂—O—(CH₂)₆—, #-(CH₂)₁—N(R_{g})—(CH₂)₁—, #-(CH₂)₁—N(R_{g})—(CH₂)₂—, #-(CH₂)₁—N(R_{g})—(CH₂)₃—, #-(CH₂)₁—N(R_{g})—(CH₂)₄—, #-(CH₂)₁—N(R_{g})—(CH₂)₅—, #-(CH₂)₁—N(R_{g})—(CH₂)₆—, #-(CH₂)₁—N(R_{g})—(CH₂)₇—, #-(CH₂)₁—N(R_{g})—(CH₂)₈—, #-(CH₂)₁—N(R_{g})—(CH₂)₉—, #-(CH₂)₁—N(R_{g})—(CH₂)₁₀—, #-(CH₂)₂—N(R_{g})—(CH₂)₁—, #-(CH₂)₂—N(R_{g})—(CH₂)₂—, #-(CH₂)₂—N(R_{g})—(CH₂)₃—, #-(CH₂)₂—N(R_{g})—(CH₂)₄—, #-(CH₂)₂—N(R_{g})—(CH₂)₅—, #-(CH₂)₂—N(R_{g})—(CH₂)₆—, #-(CH₂)₂—N(R_{g})—(CH₂)₇—, #-(CH₂)₂—N(R_{g})—(CH₂)₈—, #-(CH₂)₂—N(R_{g})—(CH₂)₉—, #-(CH₂)₂—N(R_{g})—(CH₂)₁₀—, #-(CH₂)₂—N(R_{g})—(CH₂)₁₁—, #-(CH₂)₂—N(R_{g})—(CH₂)₁₂—, #-(CH₂)₃—N(R_{g})—(CH₂)₁—, #-(CH₂)₃—N(R_{g})—(CH₂)₂—, #-(CH₂)₃—N(R_{g})—(CH₂)₃—, #-(CH₂)₄—N(R_{g})—(CH₂)₁—, #-(CH₂)₄—N(R_{g})—(CH₂)₂—, #-(CH₂)₄—N(R_{g})—(CH₂)₃—, #-(CH₂)₄—N(R_{g})—(CH₂)₄—, #-(CH₂)₅—N(R_{g})—(CH₂)₁—, #-(CH₂)₅—N(R_{g})—(CH₂)₂—, #-(CH₂)₅—N(R_{g})—(CH₂)₃—, #-(CH₂)₅—N(R_{g})—(CH₂)₄—, #-(CH₂)₅—N(R_{g})—(CH₂)₅—, #-(CH₂)₆—N(R_{g})—(CH₂)₁—, #-(CH₂)₆—N(R_{g})—(CH₂)₂—, #-(CH₂)₆—N(R_{g})—(CH₂)₃—, #-(CH₂)₇—N(R_{g})—(CH₂)₁—, #-(CH₂)₇—N(R_{g})—(CH₂)₂—, #-(CH₂)₇—N(R_{g})—(CH₂)₃—, #-(CH₂)₈—N(R_{g})—(CH₂)₁—, #-(CH₂)₈—N(R_{g})—(CH₂)₂—, #-(CH₂)₈—N(R_{g})—(CH₂)₃—, #-CH(CH₃)—N(R_{g})—(CH₂)₁—, #-CH(CH₃)—N(R_{g})—(CH₂)₂—, #-CH(CH₃)—N(R_{g})—(CH₂)₃—, #-CH(CH₃)—N(R_{g})—(CH₂)₄—, #-CH(CH₃)—N(R_{g})—(CH₂)₅—, #-CH(CH₃)—N(R_{g})—(CH₂)₆—, #-CH(CH₃)—N(R_{g})—(CH₂)₇—, #-CH(CH₃)—N(R_{g})—(CH₂)₈—, #-CH(CH₃)—N(R_{g})—(CH₂)₉—, #-CH(CH₃)—N(R_{g})—(CH₂)₁₀—, #-CH₂C(O)NHCH₂—, #-(CH₂)₂C(O)NH(CH₂)₂—, #-(CH₂)₂C(O)NH(CH₂)₃—, #-(CH₂)₂C(O)NH(CH₂)₄—, #-(CH₂)₂C(O)NH(CH₂)₅—, #-(CH₂)₃C(O)NH(CH₂)₃—, #-(CH₂)₃C(O)NH(CH₂)₄—, #-(CH₂)₄C(O)NH(CH₂)₄—, #-(CH₂)₅C(O)NH(CH₂)₅—, #-(CH₂)₆C(O)NH(CH₂)₇—, #-(CH₂)₆C(O)NH(CH₂)₆—, #-(CH₂)₇C(O)NH(CH₂)₇—, #-(CH₂)₈C(O)NH(CH₂)₈, U—(CH₂)₉C(O)NH(CH₂)₉—, #-(CH₂)₁₀C(O)NH(CH₂)₁₀—, #-(CH₂)₂C(O)NH(CH₂)₂—O—(CH₂)₂—, #-CH₂NHC(O)CH₂—, #-(CH₂)₂NHC(O)(CH₂)₂—, #-(CH₂)₂NHC(O)(CH₂)₃—, #-(CH₂)₂NHC(O)(CH₂)₄—, #-(CH₂)₂NHC(O)(CH₂)₅—, #-(CH₂)₃NHC(O)(CH₂)₃—, #-(CH₂)₃NHC(O)(CH₂)₄—, #-(CH₂)₄NHC(O)(CH₂)₄—, #-(CH₂)₅NHC(O)(CH₂)₅—, #-(CH₂)₆NHC(O)(CH₂)₇—, #-(CH₂)₆NHC(O)(CH₂)₆—, #-(CH₂)₇NHC(O)(CH₂)₇—, #-(CH₂)₈NHC(O)(CH₂)₈, #-(CH₂)₉NHC(O)(CH₂)₉—, #-(CH₂)₁₀NHC(O)(CH₂)₁₀—, #-(CH₂)₄NHC(O)(CH₂)₈—, #-(CH₂)₂NHC(O)(CH₂)₂—O—(CH₂)₂—, #-(CH₂)₄NHC(O)CH₂—, #-CH₂-piperidinylene-CH₂—, #-CH₂-piperidinylene-(CH₂)₂—, #-CH₂-piperidinylene-(CH₂)₃—, #-CH₂-piperidinylene-(CH₂)₄—, #-CH₂-piperidinylene-(CH₂)₅—, #-CH₂-piperidinylene-(CH₂)₆—, #-CH₂-piperidinylene-(CH₂)₇—, #-CH₂-piperidinylene-(CH₂)₈—, #-(CH₂)₂-piperidinylene-(CH₂)₁—, #-(CH₂)₂-piperidinylene-(CH₂)₂—, #-(CH₂)₂-piperidinylene-(CH₂)₃—, #-(CH₂)₂-piperidinylene-(CH₂)₄—, #-(CH₂)₂-piperidinylene-(CH₂)₅—, #-(CH₂)₂-piperidinylene-(CH₂)₆—, #-(CH₂)₂-piperidinylene-(CH₂)₇—, #-(CH₂)₂-piperidinylene-(CH₂)₈—, #-(CH₂)₃-piperidinylene-CH₂—, #-(CH₂)₃-piperidinylene-(CH₂)₂—, #-(CH₂)₃-piperidinylene-(CH₂)₃—, #-(CH₂)₃-piperidinylene-(CH₂)₄—, #-(CH₂)₃-piperidinylene-(CH₂)₅—, #-(CH₂)₃-piperidinylene-(CH₂)₆—, #-(CH₂)₃-piperidinylene-(CH₂)₇—, #-(CH₂)₃-piperidinylene-(CH₂)₈—, #-(CH₂)₄-piperidinylene-CH₂—, #-(CH₂)₄-piperidinylene-(CH₂)₂—, #-(CH₂)₄-piperidinylene-(CH₂)₃—, #-(CH₂)₄-piperidinylene-(CH₂)₄—, #-(CH₂)₄-piperidinylene-(CH₂)₅—, #-(CH₂)₄-piperidinylene-(CH₂)₆—, #-(CH₂)₄-piperidinylene-(CH₂)₇—, #-(CH₂)₄-piperidinylene-(CH₂)₈—, #-(CH₂)₅-piperidinylene-(CH₂)₁—, #-(CH₂)₅-piperidinylene-(CH₂)₂—, #-(CH₂)₅-piperidinylene-(CH₂)₃—, #-(CH₂)₅-piperidinylene-(CH₂)₄—, #-(CH₂)₅-piperidinylene-(CH₂)₅—, #-(CH₂)₅-piperidinylene-(CH₂)₆—, #-(CH₂)₅-piperidinylene-(CH₂)₇—, #-(CH₂)₅-piperidinylene-(CH₂)₈—, #-(CH₂)₆-piperidinylene-(CH₂)₁—, #-(CH₂)₆-piperidinylene-(CH₂)₂—, #-(CH₂)₆-piperidinylene-(CH₂)₃—, #-(CH₂)₆-piperidinylene-(CH₂)₄—, #-(CH₂)₆-piperidinylene-(CH₂)₅—, #-(CH₂)₆-piperidinylene-(CH₂)₆—, #-(CH₂)₆-piperidinylene-(CH₂)₇—, #-(CH₂)₆-piperidinylene-(CH₂)₈—, #-(CH₂)₇-piperidinylene-(CH₂)₁—, #-(CH₂)₇-piperidinylene-(CH₂)₂—, #-(CH₂)₇-piperidinylene-(CH₂)₃—, #-(CH₂)₇-piperidinylene-(CH₂)₄—, #-(CH₂)₇-piperidinylene-(CH₂)₈—, #-(CH₂)₈-piperidinylene-CH₂—, #-(CH₂)₈-piperidinylene-(CH₂)₂—, #-(CH₂)₈-piperidinylene-(CH₂)₃—, #-(CH₂)₈-piperidinylene-(CH₂)₄—, #-(CH₂)₈-piperidinylene-(CH₂)₅—, #-(CH₂)₈-piperidinylene-(CH₂)₆—, #-(CH₂)₈-piperidinylene-(CH₂)₇—, #-(CH₂)₈-piperidinylene-(CH₂)₈—, #-CH₂—N(R_{g})—CH₂-piperidinylene-CH₂—, #-CH₂—N(R_{g})—CH₂-piperidinylene-(CH₂)₂—, #-CH₂—N(R_{g})—CH₂-piperidinylene-(CH₂)₃—, #-CH₂—N(R_{g})—CH₂-piperidinylene-(CH₂)₄—, #-CH₂—N(R_{g})—CH₂-piperidinylene-(CH₂)₅—, #-CH₂—N(R_{g})—CH₂-piperidinylene-(CH₂)₆—, #-CH₂—N(R_{g})—CH₂-piperidinylene-(CH₂)₇—, #-CH₂—N(R_{g})—CH₂-piperidinylene-(CH₂)₈—, #-CH₂—N(R_{g})—(CH₂)₂-piperidinylene-CH₂—, #-CH₂—N(R_{g})—(CH₂)₂-piperidinylene-(CH₂)₂—, #-CH₂—N(R_{g})—(CH₂)₂-piperidinylene-(CH₂)₃—, #-CH₂—N(R_{g})—(CH₂)₂-piperidinylene-(CH₂)₄—, #-CH₂—N(R_{g})—(CH₂)₂-piperidinylene-(CH₂)₅—, #-CH₂—N(R_{g})—(CH₂)₂-piperidinylene-(CH₂)₆—, #-CH₂—N(R_{g})—(CH₂)₂-piperidinylene-(CH₂)₇—, #-CH₂—N(R_{g})—(CH₂)₂-piperidinylene-(CH₂)₈—, #-(CH₂)₂—N(R_{g})—CH₂-piperidinylene-CH₂—, #-(CH₂)₂—N(R_{g})—CH₂-piperidinylene-(CH₂)₂—, #-(CH₂)₂—N(R_{g})—CH₂-piperidinylene-(CH₂)₃—, #-(CH₂)₂—N(R_{g})—CH₂-piperidinylene-(CH₂)₄—, #-(CH₂)₂—N(R_{g})—CH₂-piperidinylene-(CH₂)₅—, #-(CH₂)₂—N(R_{g})—CH₂-piperidinylene-(CH₂)₆—, #-(CH₂)₂—N(R_{g})—CH₂-piperidinylene-(CH₂)₇—, #-(CH₂)₂—N(R_{g})—CH₂-piperidinylene-(CH₂)₈—, #-(CH₂)₃—N(R_{g})—CH₂-piperidinylene-CH₂—, #-(CH₂)₃—N(R_{g})—CH₂-piperidinylene-(CH₂)₂—, #-(CH₂)₃—N(R_{g})—CH₂-piperidinylene-(CH₂)₃—, #-(CH₂)₃—N(R_{g})—CH₂-piperidinylene-(CH₂)₈—, #-(CH₂)₄—N(R_{g})—CH₂-piperidinylene-CH₂—, #-(CH₂)₄—N(R_{g})—CH₂-piperidinylene-(CH₂)₂—, #-(CH₂)₄—N(R_{g})—CH₂-piperidinylene-(CH₂)₃—, #-(CH₂)₄—N(R_{g})—CH₂-piperidinylene-(CH₂)₈—, #-(CH₂)₅—N(R_{g})—CH₂-piperidinylene-(CH₂)₃—, #-(CH₂)₅—N(R_{g})—CH₂-piperidinylene-(CH₂)₈—, #-(CH₂)₆—N(R_{g})—CH₂-piperidinylene-(CH₂)₃—, #-(CH₂)₆—N(R_{g})—CH₂-piperidinylene-(CH₂)₈—, #-(CH₂)₇—N(R_{g})—CH₂-piperidinylene-(CH₂)₃—, #-(CH₂)₇—N(R_{g})—CH₂-piperidinylene-(CH₂)₈—, #-(CH₂)₈—N(R_{g})—CH₂-piperidinylene-CH₂—, #-(CH₂)₈—N(R_{g})—CH₂-piperidinylene-(CH₂)₂—, #-(CH₂)₈—N(R_{g})—CH₂-piperidinylene-(CH₂)₃—, #-(CH₂)₈—N(R_{g})—CH₂-piperidinylene-(CH₂)₄—, #-(CH₂)₈—N(R_{g})—CH₂-piperidinylene-(CH₂)₅—, #-(CH₂)₈—N(R_{g})—CH₂-piperidinylene-(CH₂)₆—, #-(CH₂)₈—N(R_{g})—CH₂-piperidinylene-(CH₂)₇—, #-(CH₂)₈—N(R_{g})—CH₂-piperidinylene-(CH₂)₈—, #-CH₂-piperazinylene-CH₂—, #-CH₂-piperazinylene-(CH₂)₂—, #-CH₂-piperazinylene-(CH₂)₃—, #-CH₂-piperazinylene-(CH₂)₄—, #-CH₂-piperazinylene-(CH₂)₅—, #-CH₂-piperazinylene-(CH₂)₆—, #-CH₂-piperazinylene-(CH₂)₇—, #-CH₂-piperazinylene-(CH₂)₈—, #-(CH₂)₂-piperazinylene-(CH₂)₁—, #-(CH₂)₂-piperazinylene-(CH₂)₂—, #-(CH₂)₂-piperazinylene-(CH₂)₃—, #-(CH₂)₂-piperazinylene-(CH₂)₄—, #-(CH₂)₂-piperazinylene-(CH₂)₅—, #-(CH₂)₂-piperazinylene-(CH₂)₆—, #-(CH₂)₂-piperazinylene-(CH₂)₇—, #-(CH₂)₂-piperazinylene-(CH₂)₈—, #-(CH₂)₃-piperazinylene-CH₂—, #-(CH₂)₃-piperazinylene-(CH₂)₂—, #-(CH₂)₃-piperazinylene-(CH₂)₃—, #-(CH₂)₃-piperazinylene-(CH₂)₄—, #-(CH₂)₃-piperazinylene-(CH₂)₅—, #-(CH₂)₃-piperazinylene-(CH₂)₆—, #-(CH₂)₃-piperazinylene-(CH₂)₇—, #-(CH₂)₃-piperazinylene-(CH₂)₈—, #-(CH₂)₄-piperazinylene-CH₂—, #-(CH₂)₄-piperazinylene-(CH₂)₂—, #-(CH₂)₄-piperazinylene-(CH₂)₃—, #-(CH₂)₄-piperazinylene-(CH₂)₄—, #-(CH₂)₄-piperazinylene-(CH₂)₅—, #-(CH₂)₄-piperazinylene-(CH₂)₆—, #-(CH₂)₄-piperazinylene-(CH₂)₇—, #-(CH₂)₄-piperazinylene-(CH₂)₈—, #-(CH₂)₅-piperazinylene-(CH₂)₁—, #-(CH₂)₅-piperazinylene-(CH₂)₂—, #-(CH₂)₅-piperazinylene-(CH₂)₃—, #-(CH₂)₅-piperazinylene-(CH₂)₄—, #-(CH₂)₅-piperazinylene-(CH₂)₅—, #-(CH₂)₅-piperazinylene-(CH₂)₆—, #-(CH₂)₅-piperazinylene-(CH₂)₇—, #-(CH₂)₅-piperazinylene-(CH₂)₈—, #-(CH₂)₆-piperazinylene-(CH₂)₁—, #-(CH₂)₆-piperazinylene-(CH₂)₂—, #-(CH₂)₆-piperazinylene-(CH₂)₃—, #-(CH₂)₆-piperazinylene-(CH₂)₄—, #-(CH₂)₆-piperazinylene-(CH₂)₅—, #-(CH₂)₆-piperazinylene-(CH₂)₆—, #-(CH₂)₆-piperazinylene-(CH₂)₇—, #-(CH₂)₆-piperazinylene-(CH₂)₈—, #-(CH₂)₇-piperazinylene-(CH₂)₁—, #-(CH₂)₇-piperazinylene-(CH₂)₂—, #-(CH₂)₇-piperazinylene-(CH₂)₃—, #-(CH₂)₇-piperazinylene-(CH₂)₄—, #-(CH₂)₇-piperazinylene-(CH₂)₈—, #-(CH₂)₈-piperazinylene-CH₂—, #-(CH₂)₈-piperazinylene-(CH₂)₂—, #-(CH₂)₈-piperazinylene-(CH₂)₃—, #-(CH₂)₈-piperazinylene-(CH₂)₄—, #-(CH₂)₈-piperazinylene-(CH₂)₅—, #-(CH₂)₈-piperazinylene-(CH₂)₆—, #-(CH₂)₈-piperazinylene-(CH₂)₇—, or #-(CH₂)₈-piperazinylene-(CH₂)₈—;
wherein the piperidinylene and the piperazinylene are each independently optionally substituted with a substituent selected from the group consisting of deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano, or any combination thereof;
each R_{g} independently represent H or C₁₋₆ alkyl;
symbol # indicates the point of attachment to U₂; and
n1, n2, n3, n4, m1, m2, and m3 each independently represent an integer of 1, 2, 3, 4, 5, 6, 7, 8, 9, 10, 11, 12, 13, 14, or 15.

31. The compound of Formula (III) or salts, enantiomers, stereoisomers, solvates, isotopically enriched analogs, prodrugs, or polymorphs thereof as claimed in claim 20, wherein the ULM represents the structure of the following formula:

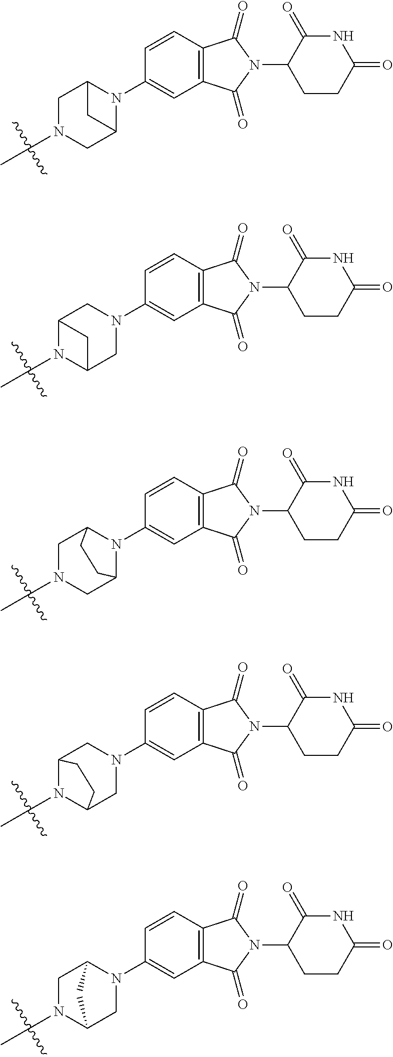 [SMILES: *N1CC2CC(C1)N2c1ccc2c(c1)C(=O)N(C1CCC(=O)NC1=O)C2=O ; *N1C2CC1CN(c1ccc3c(c1)C(=O)N(C1CCC(=O)NC1=O)C3=O)C2 ; *N1CC2CCC(C1)N2c1ccc2c(c1)C(=O)N(C1CCC(=O)NC1=O)C2=O ; *N1C2CCC1CN(c1ccc3c(c1)C(=O)N(C1CCC(=O)NC1=O)C3=O)C2 ; *N1C[C@@H]2C[C@H]1CN2c1ccc2c(c1)C(=O)N(C1CCC(=O)NC1=O)C2=O]

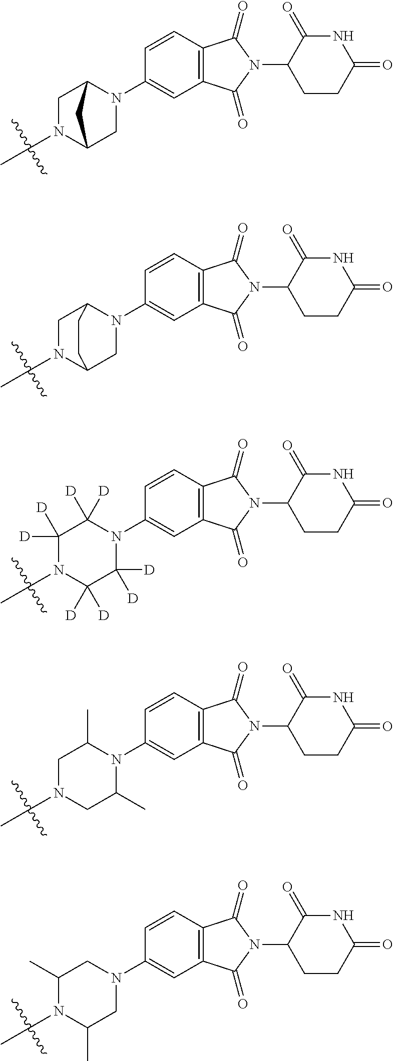 [SMILES: *N1C[C@H]2C[C@@H]1CN2c1ccc2c(c1)C(=O)N(C1CCC(=O)NC1=O)C2=O ; *N1CC2CCC1CN2c1ccc2c(c1)C(=O)N(C1CCC(=O)NC1=O)C2=O ; *N1C([2H])([2H])C([2H])([2H])N(c2ccc3c(c2)C(=O)N(C2CCC(=O)NC2=O)C3=O)C([2H])([2H])C1([2H])[2H] ; *N1CC(C)N(c2ccc3c(c2)C(=O)N(C2CCC(=O)NC2=O)C3=O)C(C)C1 ; *N1C(C)CN(c2ccc3c(c2)C(=O)N(C2CCC(=O)NC2=O)C3=O)CC1C]

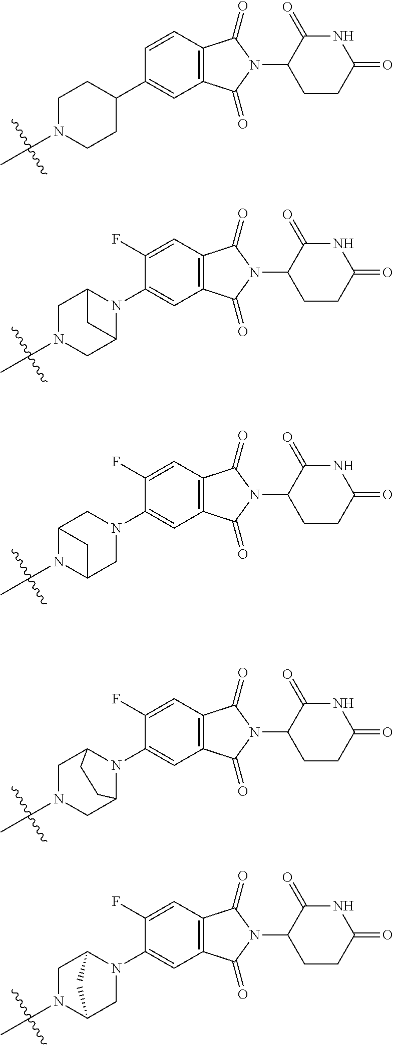 [SMILES: *N1CCC(c2ccc3c(c2)C(=O)N(C2CCC(=O)NC2=O)C3=O)CC1 ; *N1CC2CC(C1)N2c1cc2c(cc1F)C(=O)N(C1CCC(=O)NC1=O)C2=O ; *N1C2CC1CN(c1cc3c(cc1F)C(=O)N(C1CCC(=O)NC1=O)C3=O)C2 ; *N1CC2CCC(C1)N2c1cc2c(cc1F)C(=O)N(C1CCC(=O)NC1=O)C2=O ; *N1C[C@@H]2C[C@H]1CN2c1cc2c(cc1F)C(=O)N(C1CCC(=O)NC1=O)C2=O]

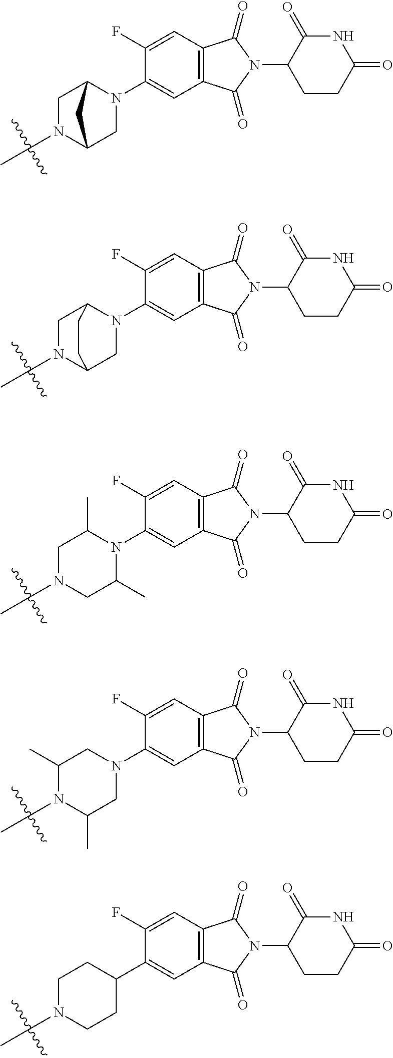 [SMILES: *N1C[C@H]2C[C@@H]1CN2c1cc2c(cc1F)C(=O)N(C1CCC(=O)NC1=O)C2=O ; *N1CC2CCC1CN2c1cc2c(cc1F)C(=O)N(C1CCC(=O)NC1=O)C2=O ; *N1CC(C)N(c2cc3c(cc2F)C(=O)N(C2CCC(=O)NC2=O)C3=O)C(C)C1 ; *N1C(C)CN(c2cc3c(cc2F)C(=O)N(C2CCC(=O)NC2=O)C3=O)CC1C ; *N1CCC(c2cc3c(cc2F)C(=O)N(C2CCC(=O)NC2=O)C3=O)CC1]

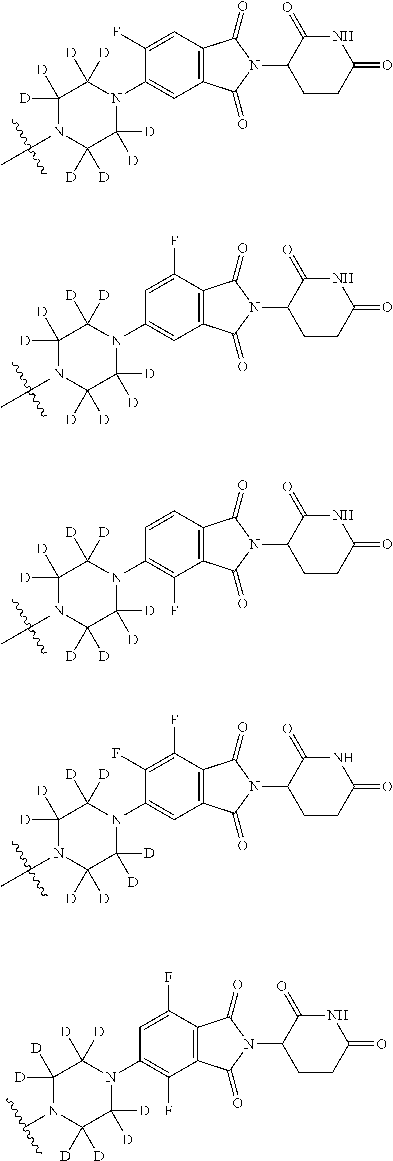 [SMILES: *N1C([2H])([2H])C([2H])([2H])N(c2cc3c(cc2F)C(=O)N(C2CCC(=O)NC2=O)C3=O)C([2H])([2H])C1([2H])[2H] ; *N1C([2H])([2H])C([2H])([2H])N(c2cc(F)c3c(c2)C(=O)N(C2CCC(=O)NC2=O)C3=O)C([2H])([2H])C1([2H])[2H] ; *N1C([2H])([2H])C([2H])([2H])N(c2ccc3c(c2F)C(=O)N(C2CCC(=O)NC2=O)C3=O)C([2H])([2H])C1([2H])[2H] ; *N1C([2H])([2H])C([2H])([2H])N(c2cc3c(c(F)c2F)C(=O)N(C2CCC(=O)NC2=O)C3=O)C([2H])([2H])C1([2H])[2H] ; *N1C([2H])([2H])C([2H])([2H])N(c2cc(F)c3c(c2F)C(=O)N(C2CCC(=O)NC2=O)C3=O)C([2H])([2H])C1([2H])[2H]]

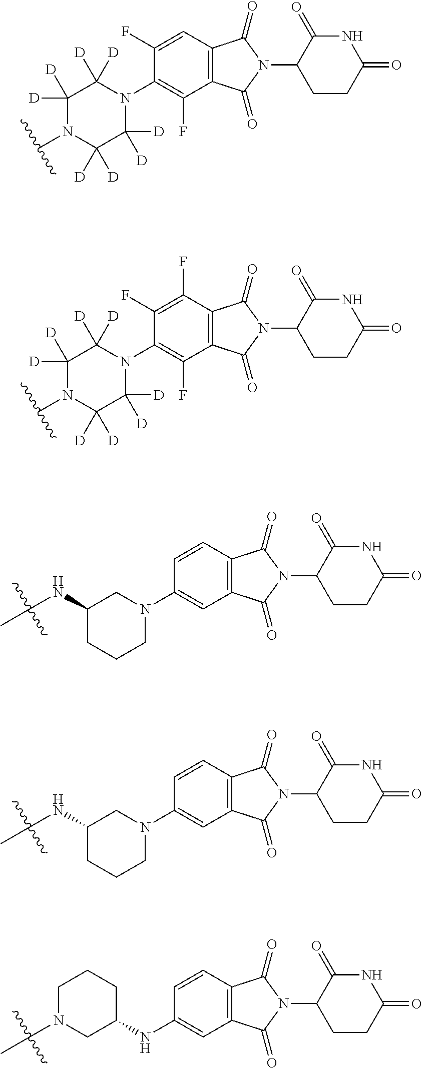 [SMILES: *N1C([2H])([2H])C([2H])([2H])N(c2c(F)cc3c(c2F)C(=O)N(C2CCC(=O)NC2=O)C3=O)C([2H])([2H])C1([2H])[2H] ; *N1C([2H])([2H])C([2H])([2H])N(c2c(F)c(F)c3c(c2F)C(=O)N(C2CCC(=O)NC2=O)C3=O)C([2H])([2H])C1([2H])[2H] ; *N[C@@H]1CCCN(c2ccc3c(c2)C(=O)N(C2CCC(=O)NC2=O)C3=O)C1 ; *N[C@H]1CCCN(c2ccc3c(c2)C(=O)N(C2CCC(=O)NC2=O)C3=O)C1 ; *N1CCC[C@H](Nc2ccc3c(c2)C(=O)N(C2CCC(=O)NC2=O)C3=O)C1]

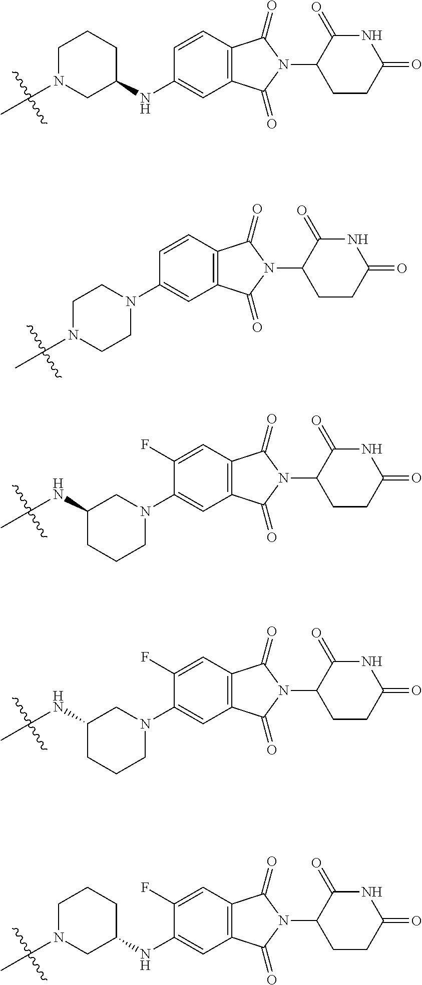 [SMILES: *N1CCC[C@@H](Nc2ccc3c(c2)C(=O)N(C2CCC(=O)NC2=O)C3=O)C1 ; *N1CCN(c2ccc3c(c2)C(=O)N(C2CCC(=O)NC2=O)C3=O)CC1 ; *N[C@@H]1CCCN(c2cc3c(cc2F)C(=O)N(C2CCC(=O)NC2=O)C3=O)C1 ; *N[C@H]1CCCN(c2cc3c(cc2F)C(=O)N(C2CCC(=O)NC2=O)C3=O)C1 ; *N1CCC[C@H](Nc2cc3c(cc2F)C(=O)N(C2CCC(=O)NC2=O)C3=O)C1]

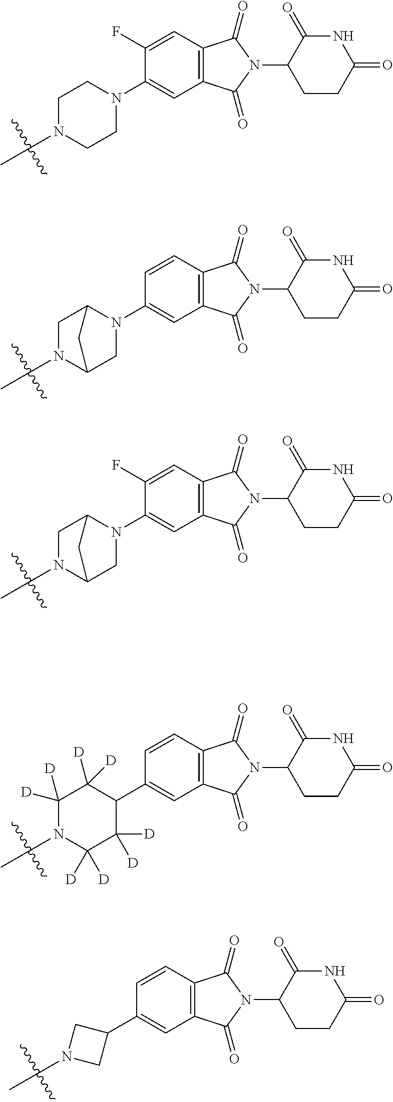 [SMILES: *N1CCN(c2cc3c(cc2F)C(=O)N(C2CCC(=O)NC2=O)C3=O)CC1 ; *N1CC2CC1CN2c1ccc2c(c1)C(=O)N(C1CCC(=O)NC1=O)C2=O ; *N1CC2CC1CN2c1cc2c(cc1F)C(=O)N(C1CCC(=O)NC1=O)C2=O ; *N1C([2H])([2H])C([2H])([2H])C(c2ccc3c(c2)C(=O)N(C2CCC(=O)NC2=O)C3=O)C([2H])([2H])C1([2H])[2H] ; *N1CC(c2ccc3c(c2)C(=O)N(C2CCC(=O)NC2=O)C3=O)C1]

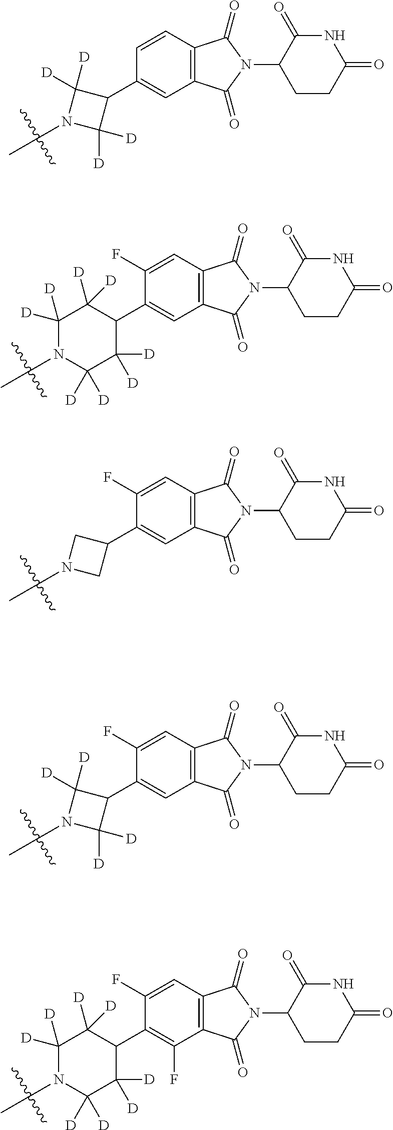 [SMILES: *N1C([2H])([2H])C(c2ccc3c(c2)C(=O)N(C2CCC(=O)NC2=O)C3=O)C1([2H])[2H] ; *N1C([2H])([2H])C([2H])([2H])C(c2cc3c(cc2F)C(=O)N(C2CCC(=O)NC2=O)C3=O)C([2H])([2H])C1([2H])[2H] ; *N1CC(c2cc3c(cc2F)C(=O)N(C2CCC(=O)NC2=O)C3=O)C1 ; *N1C([2H])([2H])C(c2cc3c(cc2F)C(=O)N(C2CCC(=O)NC2=O)C3=O)C1([2H])[2H] ; *N1C([2H])([2H])C([2H])([2H])C(c2c(F)cc3c(c2F)C(=O)N(C2CCC(=O)NC2=O)C3=O)C([2H])([2H])C1([2H])[2H]]

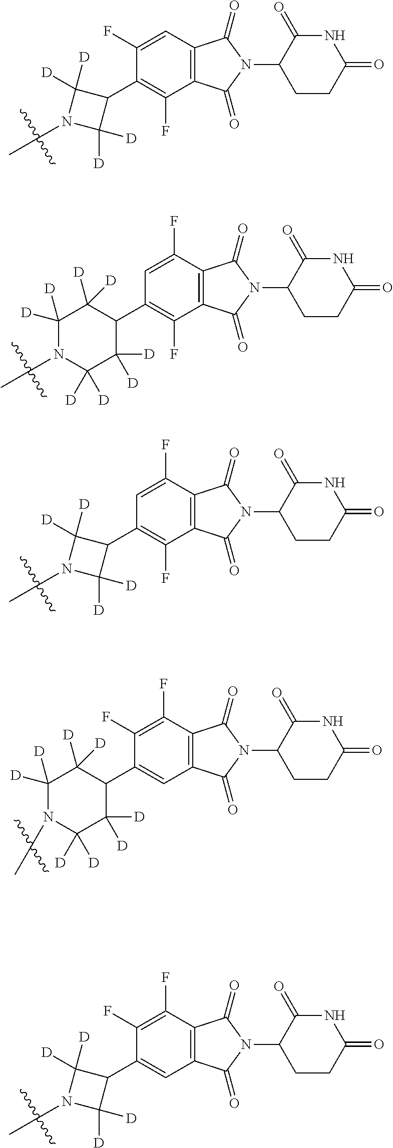 [SMILES: *N1C([2H])([2H])C(c2c(F)cc3c(c2F)C(=O)N(C2CCC(=O)NC2=O)C3=O)C1([2H])[2H] ; *N1C([2H])([2H])C([2H])([2H])C(c2cc(F)c3c(c2F)C(=O)N(C2CCC(=O)NC2=O)C3=O)C([2H])([2H])C1([2H])[2H] ; *N1C([2H])([2H])C(c2cc(F)c3c(c2F)C(=O)N(C2CCC(=O)NC2=O)C3=O)C1([2H])[2H] ; *N1C([2H])([2H])C([2H])([2H])C(c2cc3c(c(F)c2F)C(=O)N(C2CCC(=O)NC2=O)C3=O)C([2H])([2H])C1([2H])[2H] ; *N1C([2H])([2H])C(c2cc3c(c(F)c2F)C(=O)N(C2CCC(=O)NC2=O)C3=O)C1([2H])[2H]]

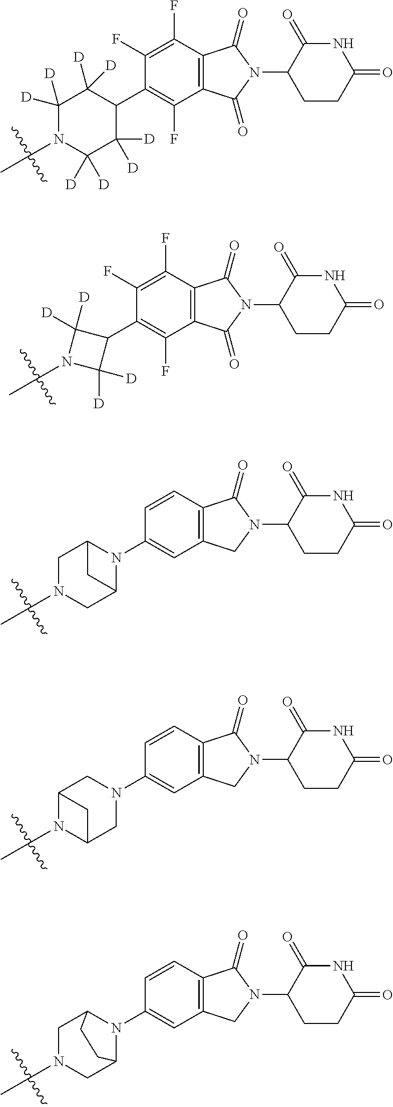 [SMILES: *N1C([2H])([2H])C([2H])([2H])C(c2c(F)c(F)c3c(c2F)C(=O)N(C2CCC(=O)NC2=O)C3=O)C([2H])([2H])C1([2H])[2H] ; *N1C([2H])([2H])C(c2c(F)c(F)c3c(c2F)C(=O)N(C2CCC(=O)NC2=O)C3=O)C1([2H])[2H] ; *N1CC2CC(C1)N2c1ccc2c(c1)CN(C1CCC(=O)NC1=O)C2=O ; *N1C2CC1CN(c1ccc3c(c1)CN(C1CCC(=O)NC1=O)C3=O)C2 ; *N1CC2CCC(C1)N2c1ccc2c(c1)CN(C1CCC(=O)NC1=O)C2=O]

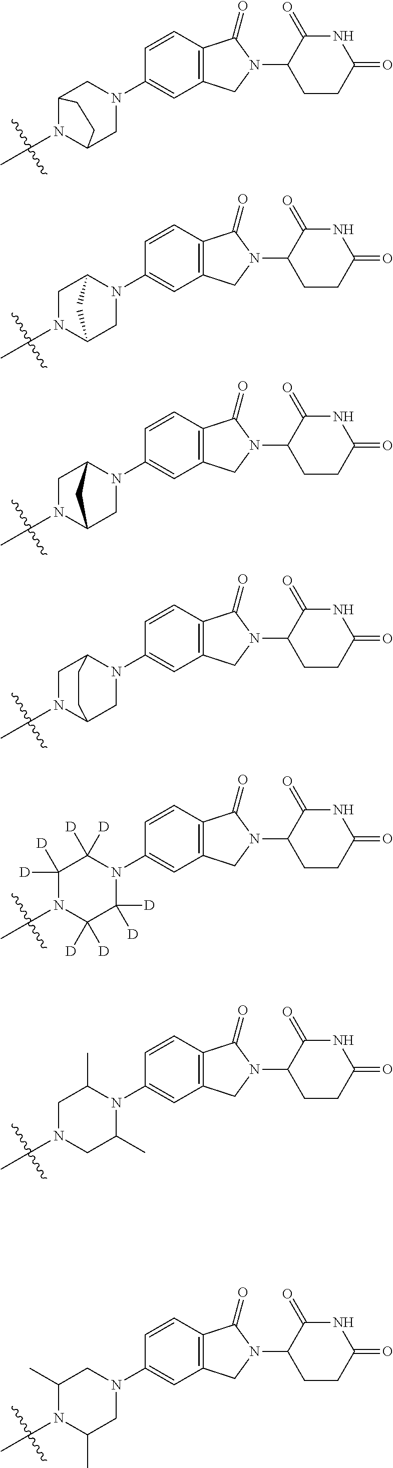

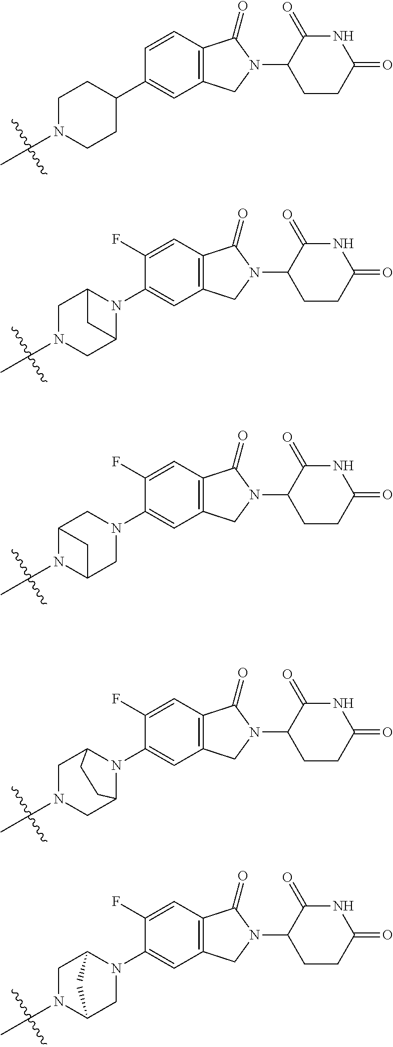 [SMILES: *N1CCC(c2ccc3c(c2)CN(C2CCC(=O)NC2=O)C3=O)CC1 ; *N1CC2CC(C1)N2c1cc2c(cc1F)C(=O)N(C1CCC(=O)NC1=O)C2 ; *N1C2CC1CN(c1cc3c(cc1F)C(=O)N(C1CCC(=O)NC1=O)C3)C2 ; *N1CC2CCC(C1)N2c1cc2c(cc1F)C(=O)N(C1CCC(=O)NC1=O)C2 ; *N1C[C@@H]2C[C@H]1CN2c1cc2c(cc1F)C(=O)N(C1CCC(=O)NC1=O)C2]

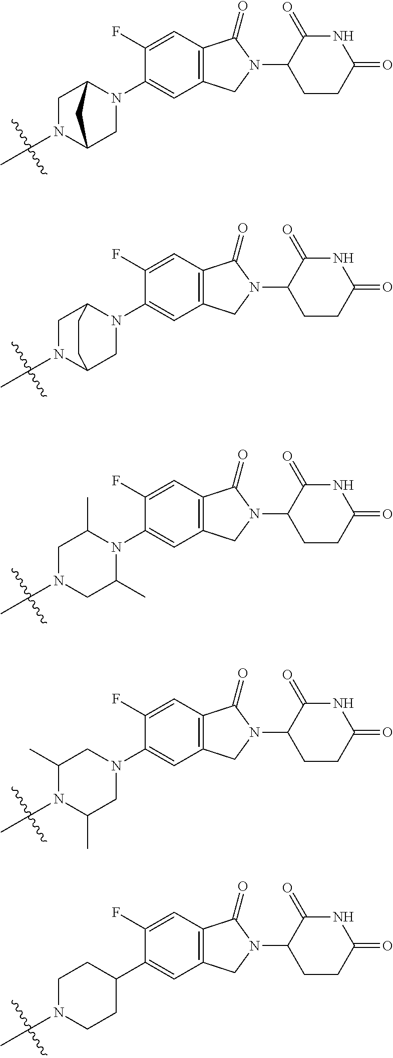 [SMILES: *N1C[C@H]2C[C@@H]1CN2c1cc2c(cc1F)C(=O)N(C1CCC(=O)NC1=O)C2 ; *N1CC2CCC1CN2c1cc2c(cc1F)C(=O)N(C1CCC(=O)NC1=O)C2 ; *N1CC(C)N(c2cc3c(cc2F)C(=O)N(C2CCC(=O)NC2=O)C3)C(C)C1 ; *N1C(C)CN(c2cc3c(cc2F)C(=O)N(C2CCC(=O)NC2=O)C3)CC1C ; *N1CCC(c2cc3c(cc2F)C(=O)N(C2CCC(=O)NC2=O)C3)CC1]

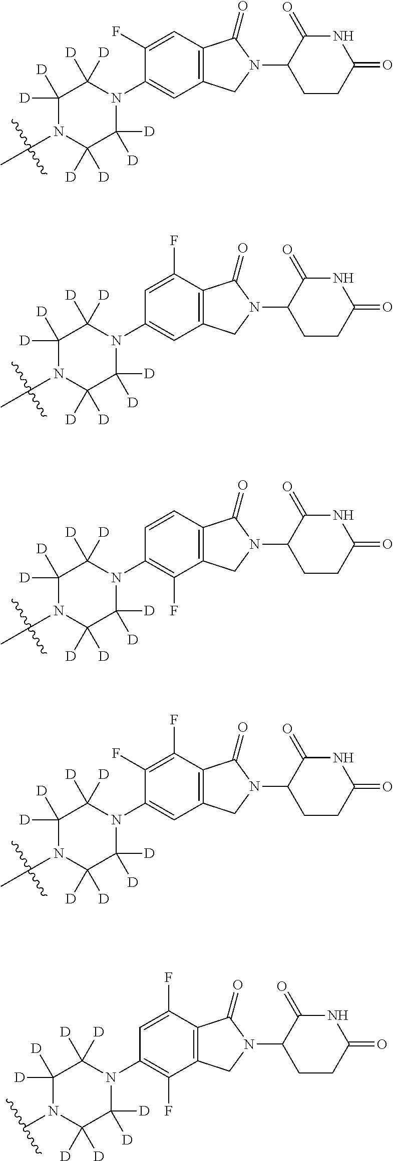 [SMILES: *N1C([2H])([2H])C([2H])([2H])N(c2cc3c(cc2F)C(=O)N(C2CCC(=O)NC2=O)C3)C([2H])([2H])C1([2H])[2H] ; *N1C([2H])([2H])C([2H])([2H])N(c2cc(F)c3c(c2)CN(C2CCC(=O)NC2=O)C3=O)C([2H])([2H])C1([2H])[2H] ; *N1C([2H])([2H])C([2H])([2H])N(c2ccc3c(c2F)CN(C2CCC(=O)NC2=O)C3=O)C([2H])([2H])C1([2H])[2H] ; *N1C([2H])([2H])C([2H])([2H])N(c2cc3c(c(F)c2F)C(=O)N(C2CCC(=O)NC2=O)C3)C([2H])([2H])C1([2H])[2H] ; *N1C([2H])([2H])C([2H])([2H])N(c2cc(F)c3c(c2F)CN(C2CCC(=O)NC2=O)C3=O)C([2H])([2H])C1([2H])[2H]]

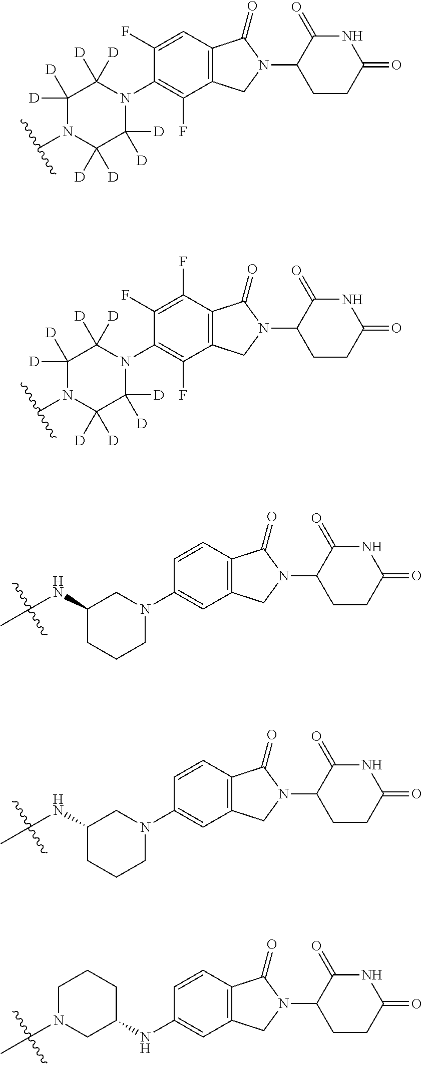 [SMILES: *N1C([2H])([2H])C([2H])([2H])N(c2c(F)cc3c(c2F)CN(C2CCC(=O)NC2=O)C3=O)C([2H])([2H])C1([2H])[2H] ; *N1C([2H])([2H])C([2H])([2H])N(c2c(F)c(F)c3c(c2F)CN(C2CCC(=O)NC2=O)C3=O)C([2H])([2H])C1([2H])[2H] ; *N[C@@H]1CCCN(c2ccc3c(c2)CN(C2CCC(=O)NC2=O)C3=O)C1 ; *N[C@H]1CCCN(c2ccc3c(c2)CN(C2CCC(=O)NC2=O)C3=O)C1 ; *N1CCC[C@H](Nc2ccc3c(c2)CN(C2CCC(=O)NC2=O)C3=O)C1]

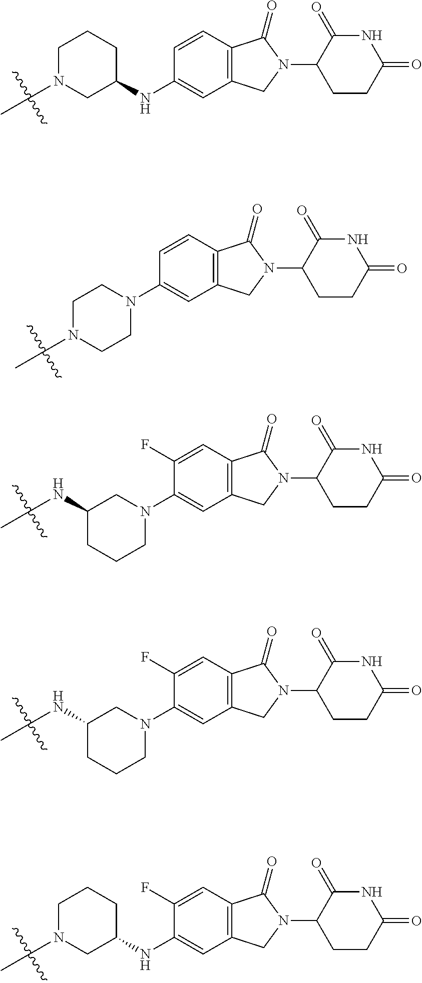 [SMILES: *N1CCC[C@@H](Nc2ccc3c(c2)CN(C2CCC(=O)NC2=O)C3=O)C1 ; *N1CCN(c2ccc3c(c2)CN(C2CCC(=O)NC2=O)C3=O)CC1 ; *N[C@@H]1CCCN(c2cc3c(cc2F)C(=O)N(C2CCC(=O)NC2=O)C3)C1 ; *N[C@H]1CCCN(c2cc3c(cc2F)C(=O)N(C2CCC(=O)NC2=O)C3)C1 ; *N1CCC[C@H](Nc2cc3c(cc2F)C(=O)N(C2CCC(=O)NC2=O)C3)C1]

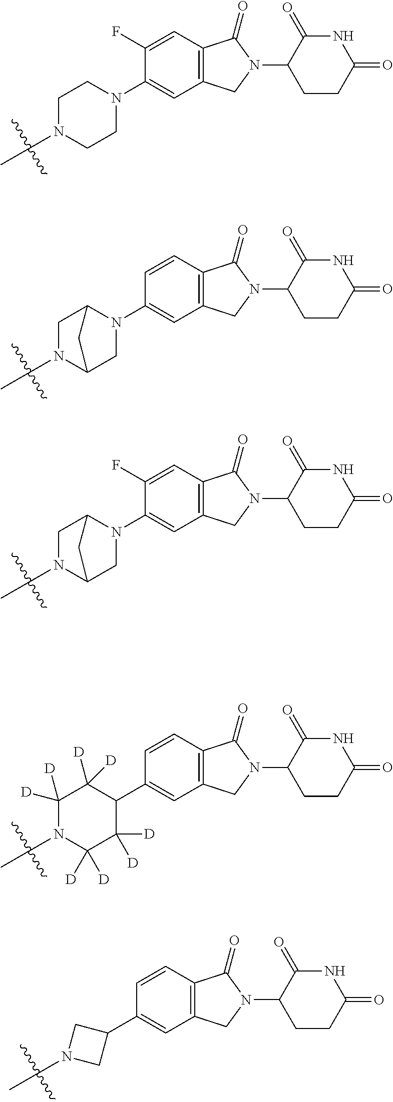 [SMILES: *N1CCN(c2cc3c(cc2F)C(=O)N(C2CCC(=O)NC2=O)C3)CC1 ; *N1CC2CC1CN2c1ccc2c(c1)CN(C1CCC(=O)NC1=O)C2=O ; *N1CC2CC1CN2c1cc2c(cc1F)C(=O)N(C1CCC(=O)NC1=O)C2 ; *N1C([2H])([2H])C([2H])([2H])C(c2ccc3c(c2)CN(C2CCC(=O)NC2=O)C3=O)C([2H])([2H])C1([2H])[2H] ; *N1CC(c2ccc3c(c2)CN(C2CCC(=O)NC2=O)C3=O)C1]

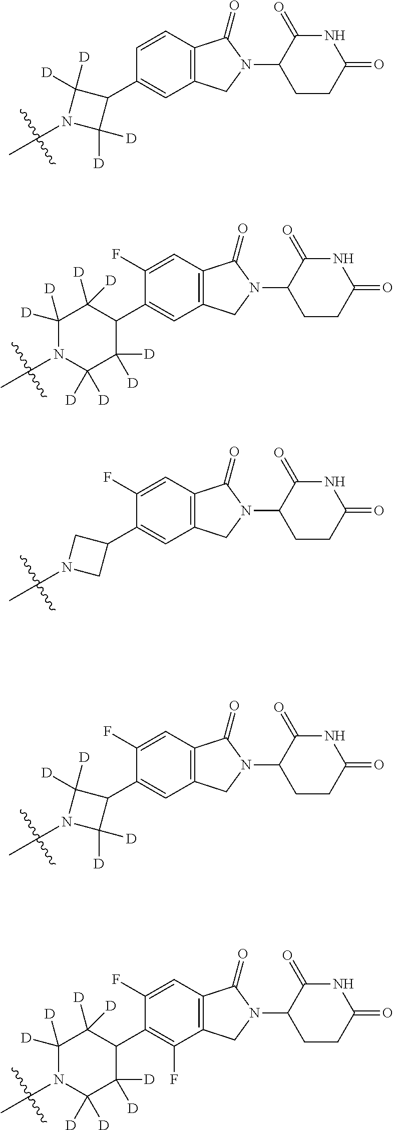 [SMILES: *N1C([2H])([2H])C(c2ccc3c(c2)CN(C2CCC(=O)NC2=O)C3=O)C1([2H])[2H] ; *N1C([2H])([2H])C([2H])([2H])C(c2cc3c(cc2F)C(=O)N(C2CCC(=O)NC2=O)C3)C([2H])([2H])C1([2H])[2H] ; *N1CC(c2cc3c(cc2F)C(=O)N(C2CCC(=O)NC2=O)C3)C1 ; *N1C([2H])([2H])C(c2cc3c(cc2F)C(=O)N(C2CCC(=O)NC2=O)C3)C1([2H])[2H] ; *N1C([2H])([2H])C([2H])([2H])C(c2c(F)cc3c(c2F)CN(C2CCC(=O)NC2=O)C3=O)C([2H])([2H])C1([2H])[2H]]

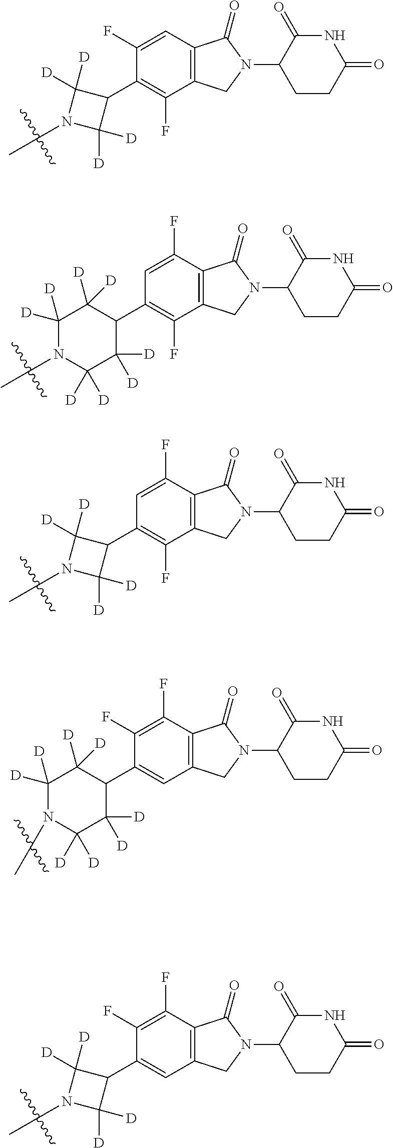 [SMILES: *N1C([2H])([2H])C(c2c(F)cc3c(c2F)CN(C2CCC(=O)NC2=O)C3=O)C1([2H])[2H] ; *N1C([2H])([2H])C([2H])([2H])C(c2cc(F)c3c(c2F)CN(C2CCC(=O)NC2=O)C3=O)C([2H])([2H])C1([2H])[2H] ; *N1C([2H])([2H])C(c2cc(F)c3c(c2F)CN(C2CCC(=O)NC2=O)C3=O)C1([2H])[2H] ; *N1C([2H])([2H])C([2H])([2H])C(c2cc3c(c(F)c2F)C(=O)N(C2CCC(=O)NC2=O)C3)C([2H])([2H])C1([2H])[2H] ; *N1C([2H])([2H])C(c2cc3c(c(F)c2F)C(=O)N(C2CCC(=O)NC2=O)C3)C1([2H])[2H]]

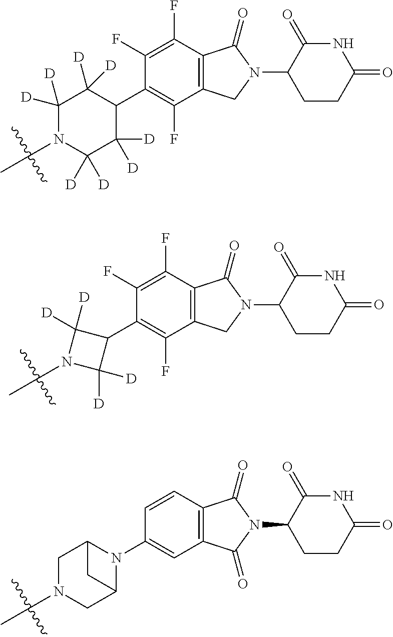 [SMILES: *N1C([2H])([2H])C([2H])([2H])C(c2c(F)c(F)c3c(c2F)CN(C2CCC(=O)NC2=O)C3=O)C([2H])([2H])C1([2H])[2H] ; *N1C([2H])([2H])C(c2c(F)c(F)c3c(c2F)CN(C2CCC(=O)NC2=O)C3=O)C1([2H])[2H] ; *N1CC2CC(C1)N2c1ccc2c(c1)C(=O)N([C@@H]1CCC(=O)NC1=O)C2=O]

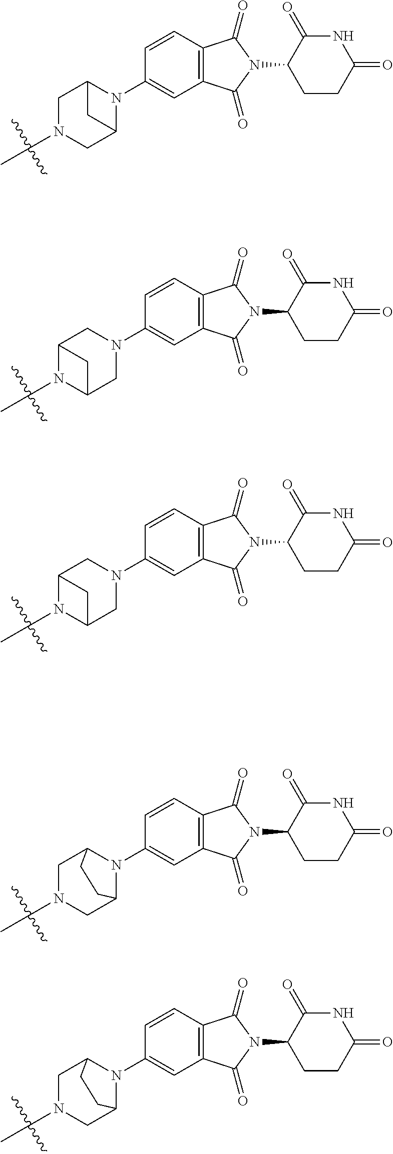 [SMILES: *N1CC2CC(C1)N2c1ccc2c(c1)C(=O)N([C@H]1CCC(=O)NC1=O)C2=O ; *N1C2CC1CN(c1ccc3c(c1)C(=O)N([C@@H]1CCC(=O)NC1=O)C3=O)C2 ; *N1C2CC1CN(c1ccc3c(c1)C(=O)N([C@H]1CCC(=O)NC1=O)C3=O)C2 ; *N1CC2CCC(C1)N2c1ccc2c(c1)C(=O)N([C@@H]1CCC(=O)NC1=O)C2=O ; *N1CC2CCC(C1)N2c1ccc2c(c1)C(=O)N([C@@H]1CCC(=O)NC1=O)C2=O]

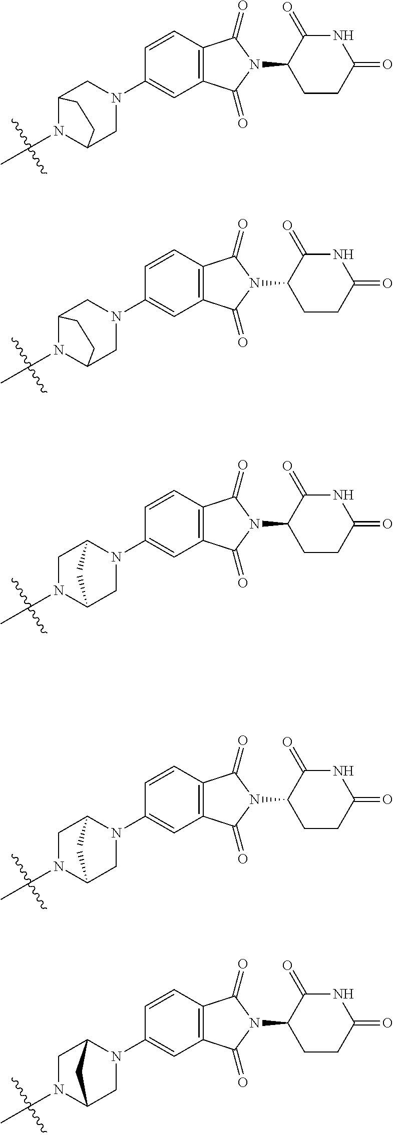 [SMILES: *N1C2CCC1CN(c1ccc3c(c1)C(=O)N([C@@H]1CCC(=O)NC1=O)C3=O)C2 ; *N1C2CCC1CN(c1ccc3c(c1)C(=O)N([C@H]1CCC(=O)NC1=O)C3=O)C2 ; *N1C[C@@H]2C[C@H]1CN2c1ccc2c(c1)C(=O)N([C@@H]1CCC(=O)NC1=O)C2=O ; *N1C[C@@H]2C[C@H]1CN2c1ccc2c(c1)C(=O)N([C@H]1CCC(=O)NC1=O)C2=O ; *N1C[C@H]2C[C@@H]1CN2c1ccc2c(c1)C(=O)N([C@@H]1CCC(=O)NC1=O)C2=O]

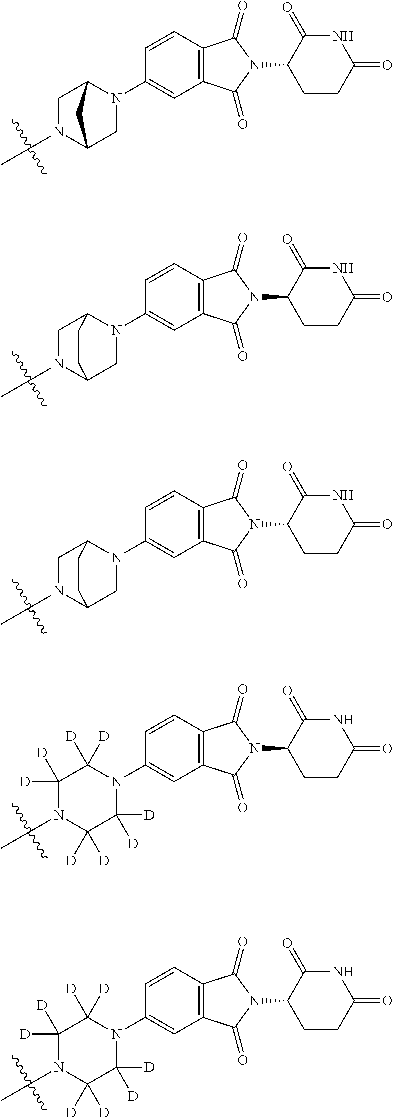 [SMILES: *N1C[C@H]2C[C@@H]1CN2c1ccc2c(c1)C(=O)N([C@H]1CCC(=O)NC1=O)C2=O ; *N1CC2CCC1CN2c1ccc2c(c1)C(=O)N([C@@H]1CCC(=O)NC1=O)C2=O ; *N1CC2CCC1CN2c1ccc2c(c1)C(=O)N([C@H]1CCC(=O)NC1=O)C2=O ; *N1C([2H])([2H])C([2H])([2H])N(c2ccc3c(c2)C(=O)N([C@@H]2CCC(=O)NC2=O)C3=O)C([2H])([2H])C1([2H])[2H] ; *N1C([2H])([2H])C([2H])([2H])N(c2ccc3c(c2)C(=O)N([C@H]2CCC(=O)NC2=O)C3=O)C([2H])([2H])C1([2H])[2H]]

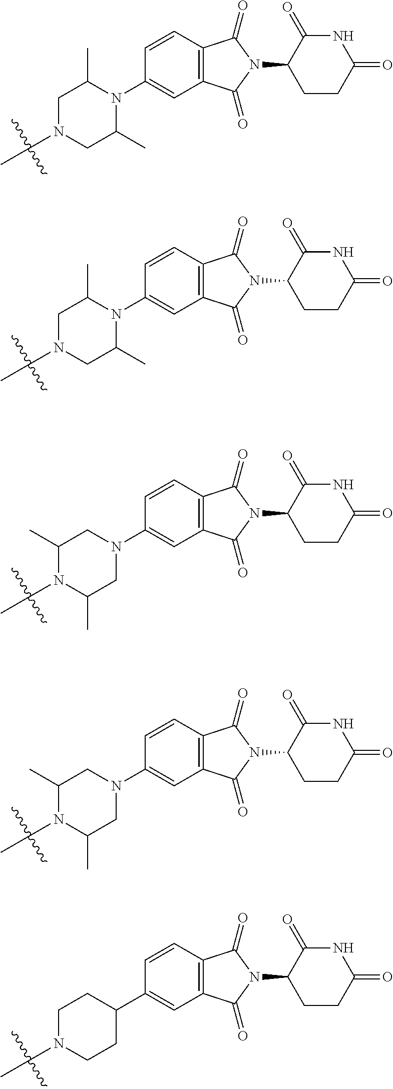 [SMILES: *N1CC(C)N(c2ccc3c(c2)C(=O)N([C@@H]2CCC(=O)NC2=O)C3=O)C(C)C1 ; *N1CC(C)N(c2ccc3c(c2)C(=O)N([C@H]2CCC(=O)NC2=O)C3=O)C(C)C1 ; *N1C(C)CN(c2ccc3c(c2)C(=O)N([C@@H]2CCC(=O)NC2=O)C3=O)CC1C ; *N1C(C)CN(c2ccc3c(c2)C(=O)N([C@H]2CCC(=O)NC2=O)C3=O)CC1C ; *N1CCC(c2ccc3c(c2)C(=O)N([C@@H]2CCC(=O)NC2=O)C3=O)CC1]

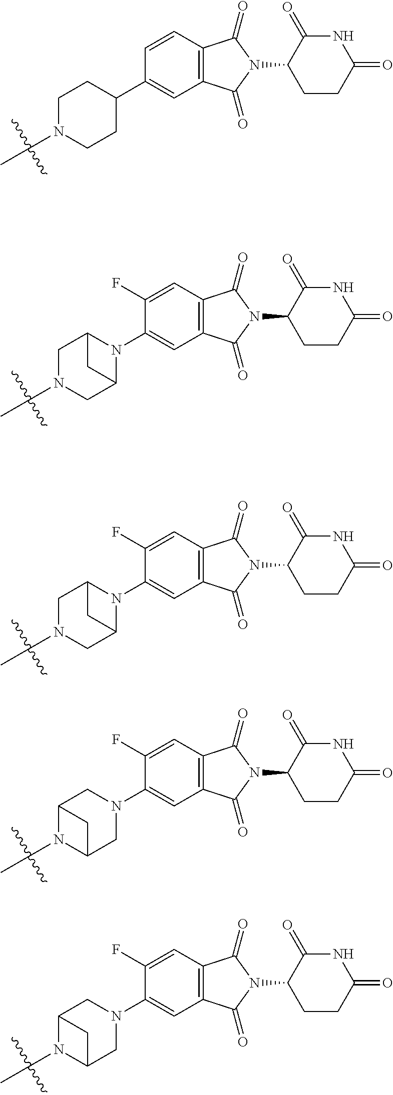 [SMILES: *N1CCC(c2ccc3c(c2)C(=O)N([C@H]2CCC(=O)NC2=O)C3=O)CC1 ; *N1CC2CC(C1)N2c1cc2c(cc1F)C(=O)N([C@@H]1CCC(=O)NC1=O)C2=O ; *N1CC2CC(C1)N2c1cc2c(cc1F)C(=O)N([C@H]1CCC(=O)NC1=O)C2=O ; *N1C2CC1CN(c1cc3c(cc1F)C(=O)N([C@@H]1CCC(=O)NC1=O)C3=O)C2 ; *N1C2CC1CN(c1cc3c(cc1F)C(=O)N([C@H]1CCC(=O)NC1=O)C3=O)C2]

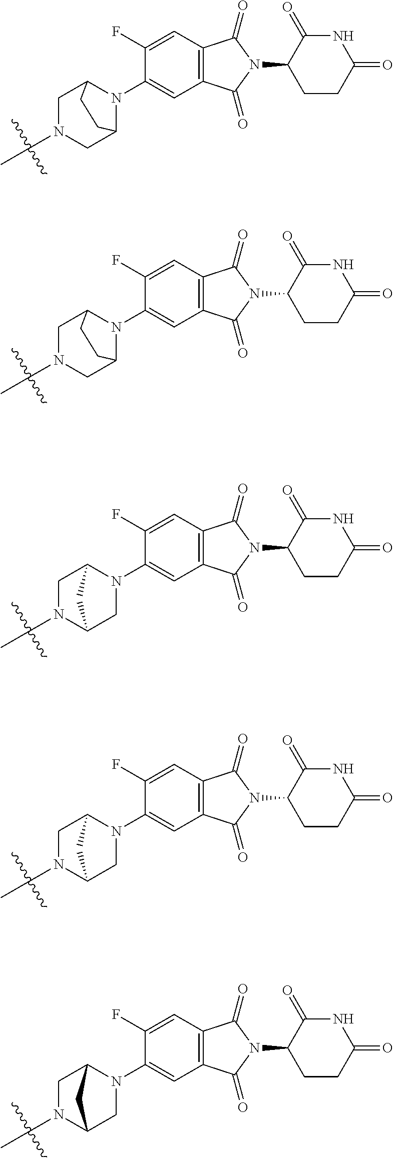 [SMILES: *N1CC2CCC(C1)N2c1cc2c(cc1F)C(=O)N([C@@H]1CCC(=O)NC1=O)C2=O ; *N1CC2CCC(C1)N2c1cc2c(cc1F)C(=O)N([C@H]1CCC(=O)NC1=O)C2=O ; *N1C[C@@H]2C[C@H]1CN2c1cc2c(cc1F)C(=O)N([C@@H]1CCC(=O)NC1=O)C2=O ; *N1C[C@@H]2C[C@H]1CN2c1cc2c(cc1F)C(=O)N([C@H]1CCC(=O)NC1=O)C2=O ; *N1C[C@H]2C[C@@H]1CN2c1cc2c(cc1F)C(=O)N([C@@H]1CCC(=O)NC1=O)C2=O]

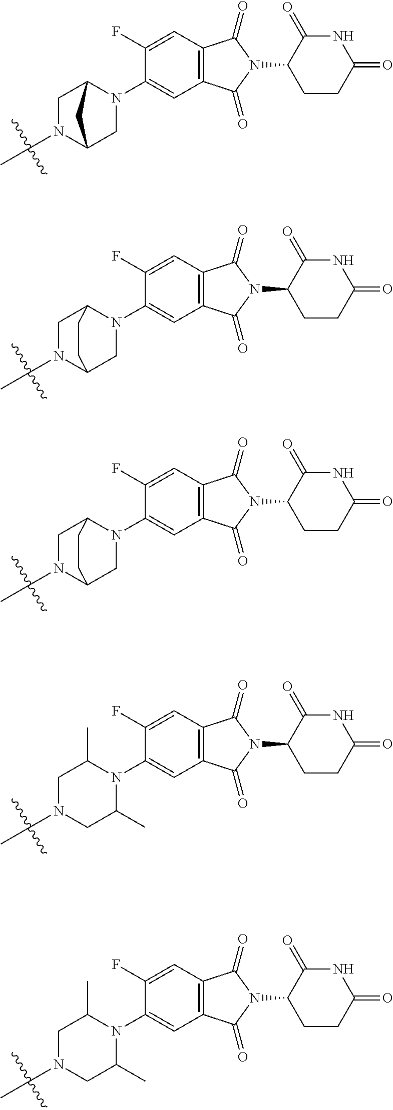 [SMILES: *N1C[C@H]2C[C@@H]1CN2c1cc2c(cc1F)C(=O)N([C@H]1CCC(=O)NC1=O)C2=O ; *N1CC2CCC1CN2c1cc2c(cc1F)C(=O)N([C@@H]1CCC(=O)NC1=O)C2=O ; *N1CC2CCC1CN2c1cc2c(cc1F)C(=O)N([C@H]1CCC(=O)NC1=O)C2=O ; *N1CC(C)N(c2cc3c(cc2F)C(=O)N([C@@H]2CCC(=O)NC2=O)C3=O)C(C)C1 ; *N1CC(C)N(c2cc3c(cc2F)C(=O)N([C@H]2CCC(=O)NC2=O)C3=O)C(C)C1]

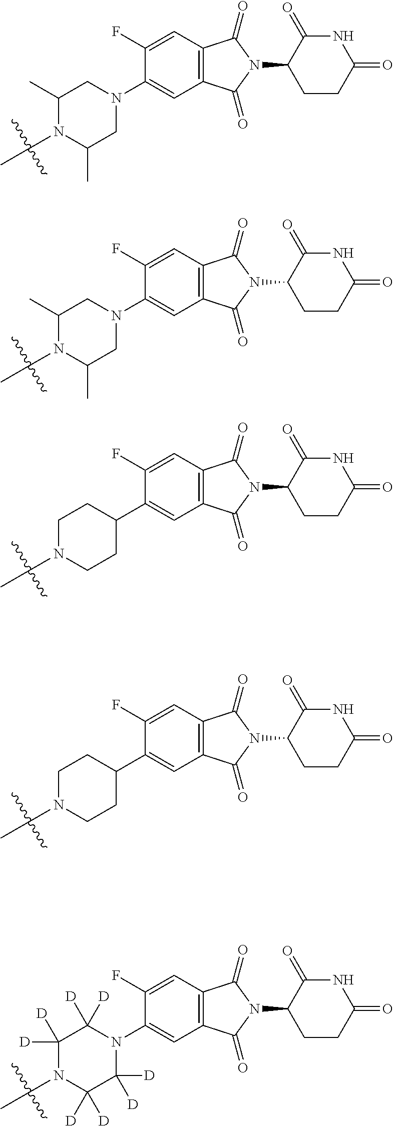 [SMILES: *N1C(C)CN(c2cc3c(cc2F)C(=O)N([C@@H]2CCC(=O)NC2=O)C3=O)CC1C ; *N1C(C)CN(c2cc3c(cc2F)C(=O)N([C@H]2CCC(=O)NC2=O)C3=O)CC1C ; *N1CCC(c2cc3c(cc2F)C(=O)N([C@@H]2CCC(=O)NC2=O)C3=O)CC1 ; *N1CCC(c2cc3c(cc2F)C(=O)N([C@H]2CCC(=O)NC2=O)C3=O)CC1 ; *N1C([2H])([2H])C([2H])([2H])N(c2cc3c(cc2F)C(=O)N([C@@H]2CCC(=O)NC2=O)C3=O)C([2H])([2H])C1([2H])[2H]]

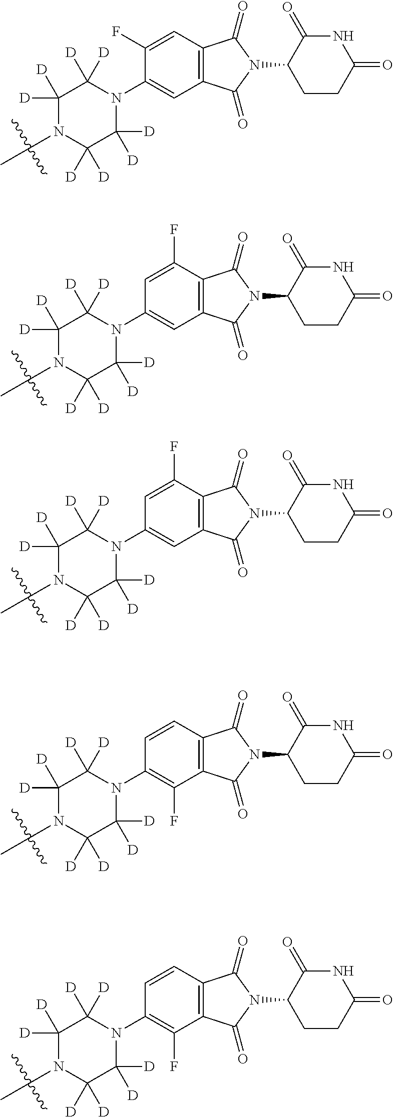

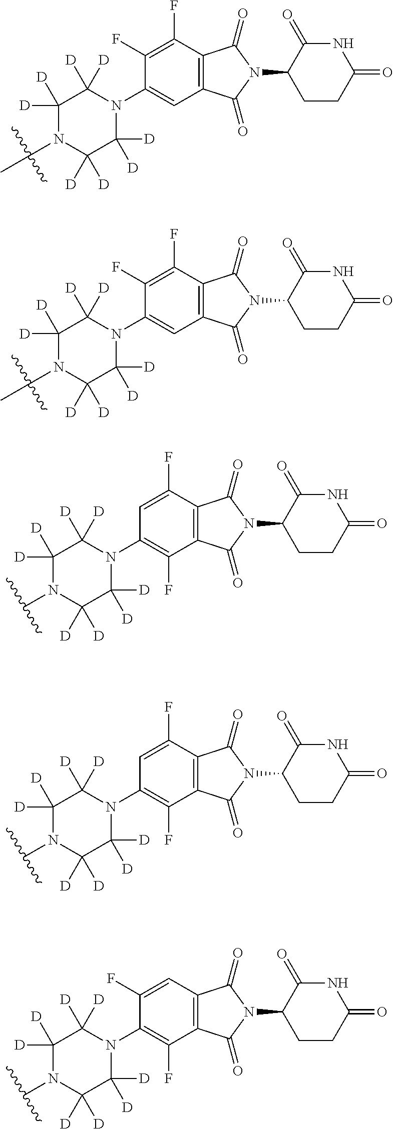

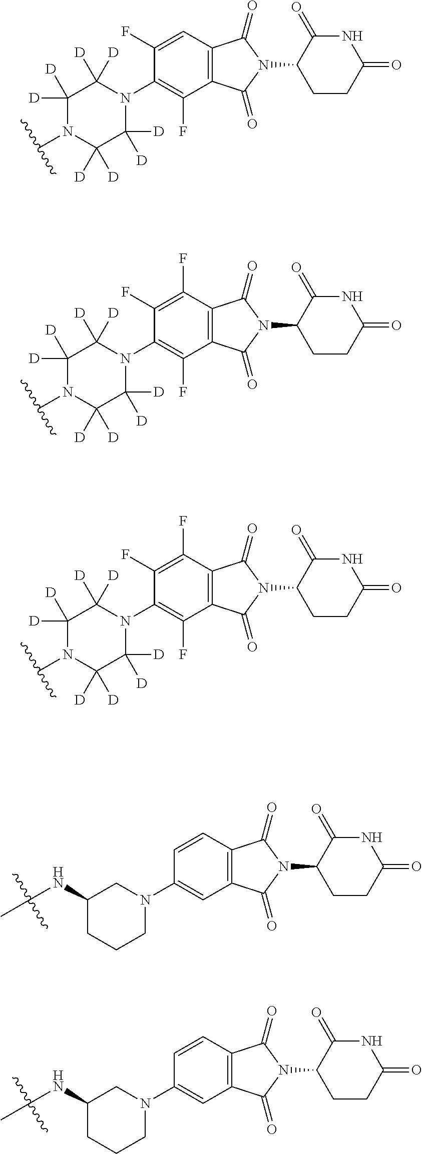 [SMILES: *N1C([2H])([2H])C([2H])([2H])N(c2c(F)cc3c(c2F)C(=O)N([C@H]2CCC(=O)NC2=O)C3=O)C([2H])([2H])C1([2H])[2H] ; *N1C([2H])([2H])C([2H])([2H])N(c2c(F)c(F)c3c(c2F)C(=O)N([C@@H]2CCC(=O)NC2=O)C3=O)C([2H])([2H])C1([2H])[2H] ; *N1C([2H])([2H])C([2H])([2H])N(c2c(F)c(F)c3c(c2F)C(=O)N([C@H]2CCC(=O)NC2=O)C3=O)C([2H])([2H])C1([2H])[2H] ; *N[C@@H]1CCCN(c2ccc3c(c2)C(=O)N([C@@H]2CCC(=O)NC2=O)C3=O)C1 ; *N[C@@H]1CCCN(c2ccc3c(c2)C(=O)N([C@H]2CCC(=O)NC2=O)C3=O)C1]

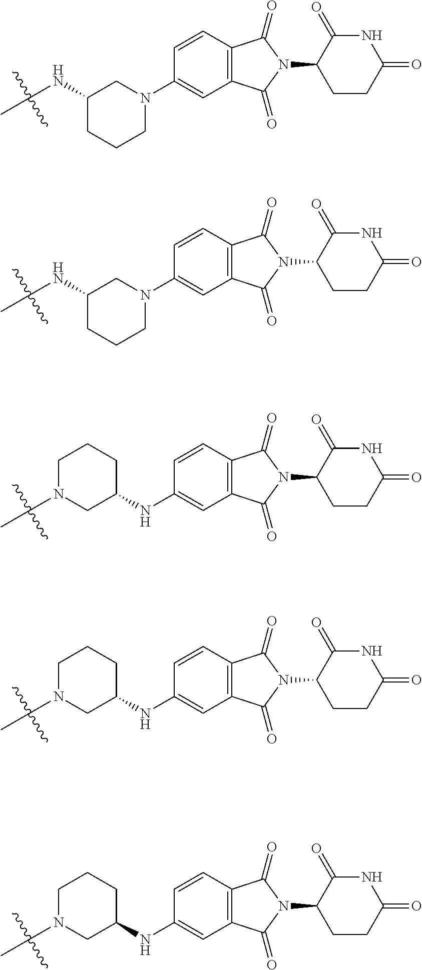 [SMILES: *N[C@H]1CCCN(c2ccc3c(c2)C(=O)N([C@@H]2CCC(=O)NC2=O)C3=O)C1 ; *N[C@H]1CCCN(c2ccc3c(c2)C(=O)N([C@H]2CCC(=O)NC2=O)C3=O)C1 ; *N1CCC[C@H](Nc2ccc3c(c2)C(=O)N([C@@H]2CCC(=O)NC2=O)C3=O)C1 ; *N1CCC[C@H](Nc2ccc3c(c2)C(=O)N([C@H]2CCC(=O)NC2=O)C3=O)C1 ; *N1CCC[C@@H](Nc2ccc3c(c2)C(=O)N([C@@H]2CCC(=O)NC2=O)C3=O)C1]

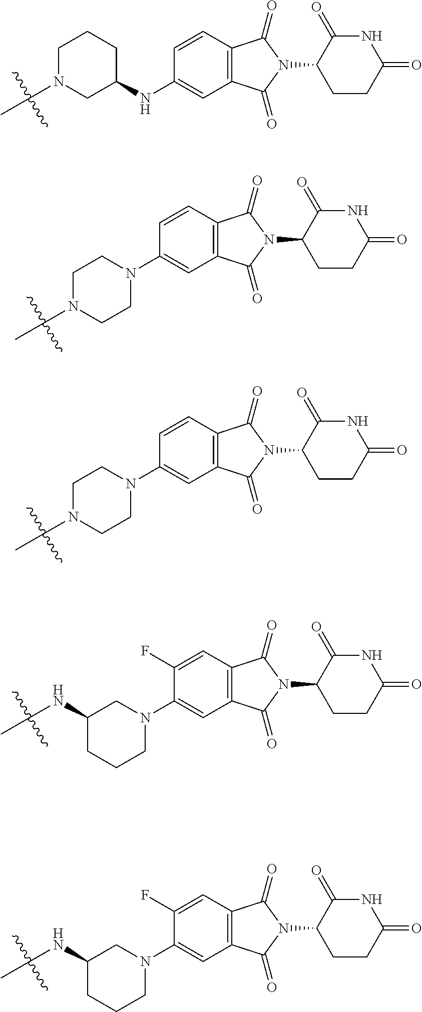 [SMILES: *N1CCC[C@@H](Nc2ccc3c(c2)C(=O)N([C@H]2CCC(=O)NC2=O)C3=O)C1 ; *N1CCN(c2ccc3c(c2)C(=O)N([C@@H]2CCC(=O)NC2=O)C3=O)CC1 ; *N1CCN(c2ccc3c(c2)C(=O)N([C@H]2CCC(=O)NC2=O)C3=O)CC1 ; *N[C@@H]1CCCN(c2cc3c(cc2F)C(=O)N([C@@H]2CCC(=O)NC2=O)C3=O)C1 ; *N[C@@H]1CCCN(c2cc3c(cc2F)C(=O)N([C@H]2CCC(=O)NC2=O)C3=O)C1]

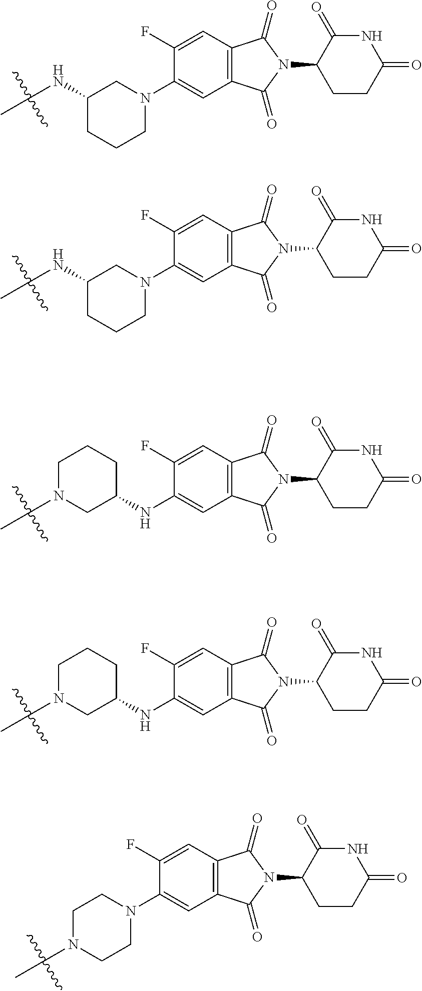 [SMILES: *N[C@H]1CCCN(c2cc3c(cc2F)C(=O)N([C@@H]2CCC(=O)NC2=O)C3=O)C1 ; *N[C@H]1CCCN(c2cc3c(cc2F)C(=O)N([C@H]2CCC(=O)NC2=O)C3=O)C1 ; *N1CCC[C@H](Nc2cc3c(cc2F)C(=O)N([C@@H]2CCC(=O)NC2=O)C3=O)C1 ; *N1CCC[C@H](Nc2cc3c(cc2F)C(=O)N([C@H]2CCC(=O)NC2=O)C3=O)C1 ; *N1CCN(c2cc3c(cc2F)C(=O)N([C@@H]2CCC(=O)NC2=O)C3=O)CC1]

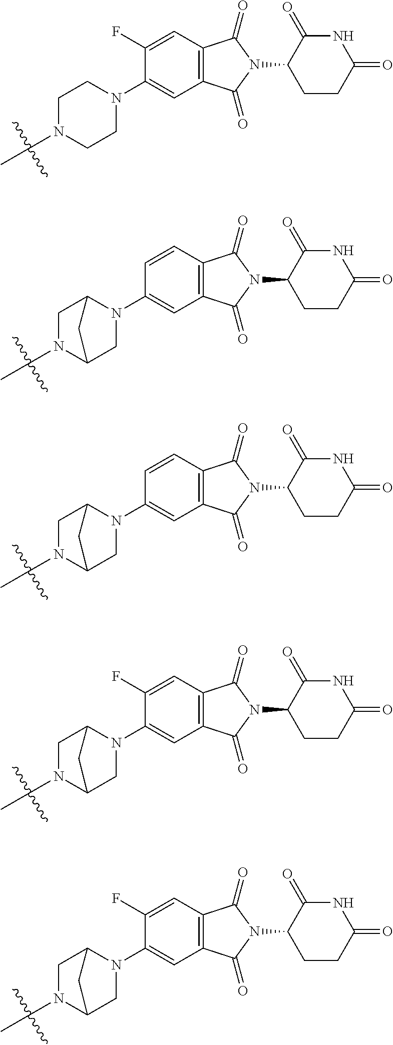 [SMILES: *N1CCN(c2cc3c(cc2F)C(=O)N([C@H]2CCC(=O)NC2=O)C3=O)CC1 ; *N1CC2CC1CN2c1ccc2c(c1)C(=O)N([C@@H]1CCC(=O)NC1=O)C2=O ; *N1CC2CC1CN2c1ccc2c(c1)C(=O)N([C@H]1CCC(=O)NC1=O)C2=O ; *N1CC2CC1CN2c1cc2c(cc1F)C(=O)N([C@@H]1CCC(=O)NC1=O)C2=O ; *N1CC2CC1CN2c1cc2c(cc1F)C(=O)N([C@H]1CCC(=O)NC1=O)C2=O]

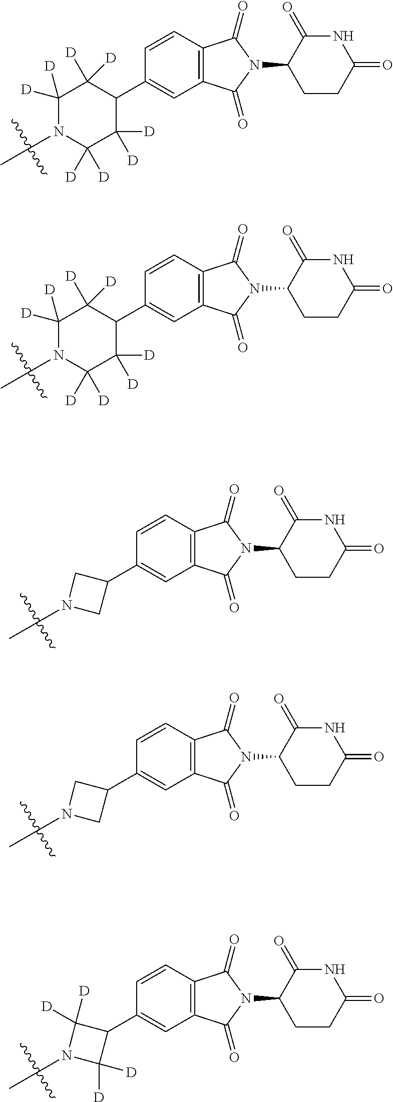 [SMILES: *N1C([2H])([2H])C([2H])([2H])C(c2ccc3c(c2)C(=O)N([C@@H]2CCC(=O)NC2=O)C3=O)C([2H])([2H])C1([2H])[2H] ; *N1C([2H])([2H])C([2H])([2H])C(c2ccc3c(c2)C(=O)N([C@H]2CCC(=O)NC2=O)C3=O)C([2H])([2H])C1([2H])[2H] ; *N1CC(c2ccc3c(c2)C(=O)N([C@@H]2CCC(=O)NC2=O)C3=O)C1 ; *N1CC(c2ccc3c(c2)C(=O)N([C@H]2CCC(=O)NC2=O)C3=O)C1 ; *N1C([2H])([2H])C(c2ccc3c(c2)C(=O)N([C@@H]2CCC(=O)NC2=O)C3=O)C1([2H])[2H]]

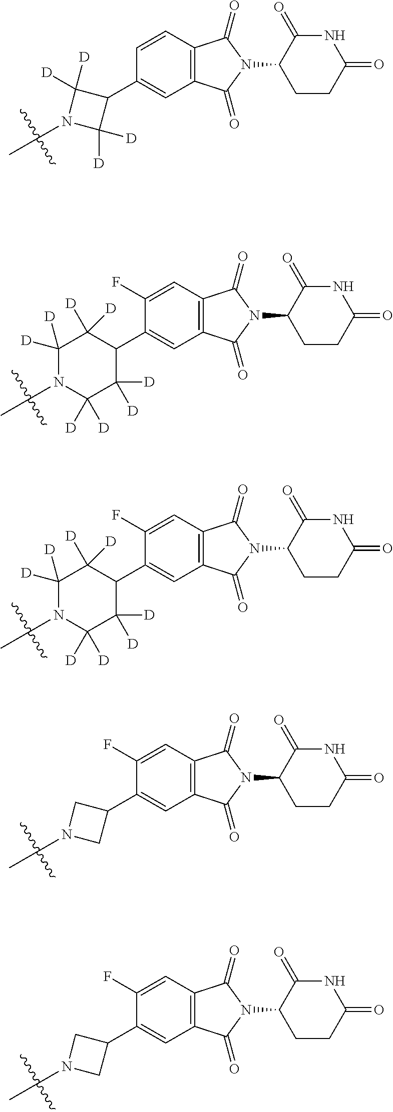 [SMILES: *N1C([2H])([2H])C(c2ccc3c(c2)C(=O)N([C@H]2CCC(=O)NC2=O)C3=O)C1([2H])[2H] ; *N1C([2H])([2H])C([2H])([2H])C(c2cc3c(cc2F)C(=O)N([C@@H]2CCC(=O)NC2=O)C3=O)C([2H])([2H])C1([2H])[2H] ; *N1C([2H])([2H])C([2H])([2H])C(c2cc3c(cc2F)C(=O)N([C@H]2CCC(=O)NC2=O)C3=O)C([2H])([2H])C1([2H])[2H] ; *N1CC(c2cc3c(cc2F)C(=O)N([C@@H]2CCC(=O)NC2=O)C3=O)C1 ; *N1CC(c2cc3c(cc2F)C(=O)N([C@H]2CCC(=O)NC2=O)C3=O)C1]

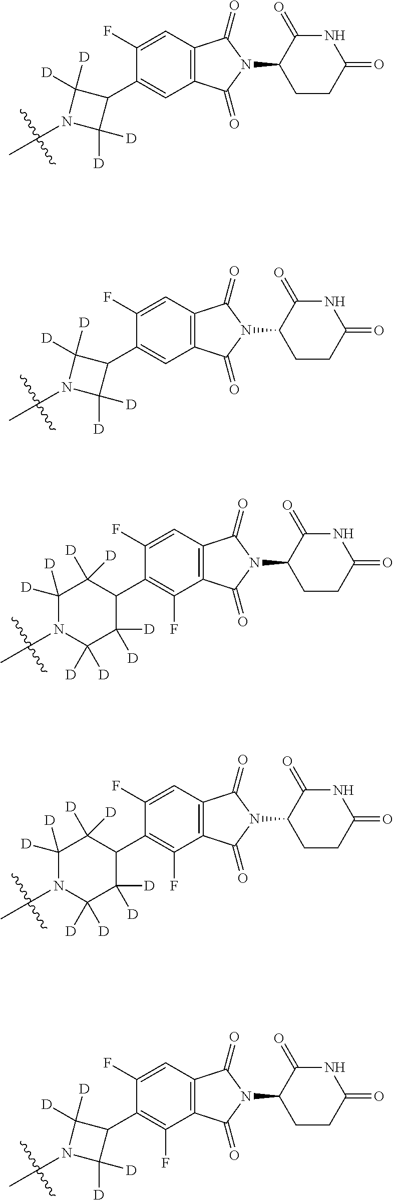 [SMILES: *N1C([2H])([2H])C(c2cc3c(cc2F)C(=O)N([C@@H]2CCC(=O)NC2=O)C3=O)C1([2H])[2H] ; *N1C([2H])([2H])C(c2cc3c(cc2F)C(=O)N([C@H]2CCC(=O)NC2=O)C3=O)C1([2H])[2H] ; *N1C([2H])([2H])C([2H])([2H])C(c2c(F)cc3c(c2F)C(=O)N([C@@H]2CCC(=O)NC2=O)C3=O)C([2H])([2H])C1([2H])[2H] ; *N1C([2H])([2H])C([2H])([2H])C(c2c(F)cc3c(c2F)C(=O)N([C@H]2CCC(=O)NC2=O)C3=O)C([2H])([2H])C1([2H])[2H] ; *N1C([2H])([2H])C(c2c(F)cc3c(c2F)C(=O)N([C@@H]2CCC(=O)NC2=O)C3=O)C1([2H])[2H]]

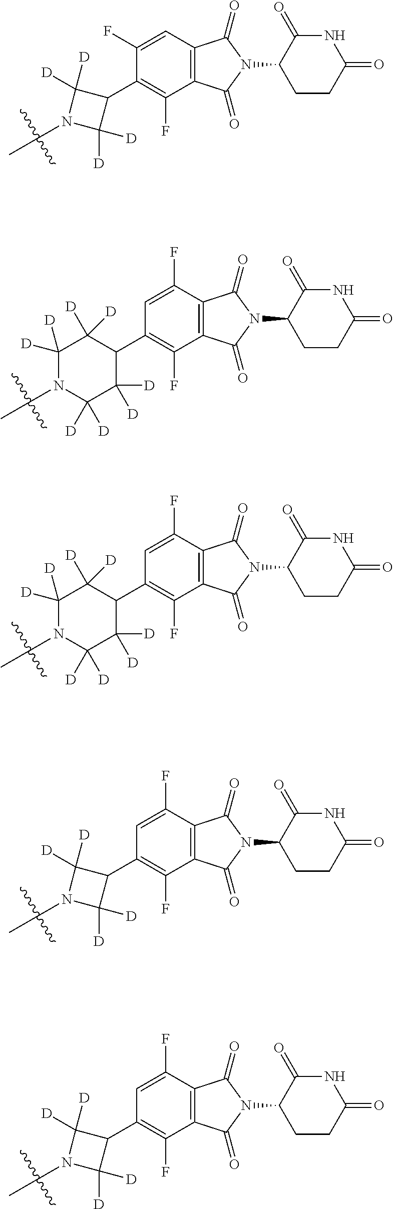 [SMILES: *N1C([2H])([2H])C(c2c(F)cc3c(c2F)C(=O)N([C@H]2CCC(=O)NC2=O)C3=O)C1([2H])[2H] ; *N1C([2H])([2H])C([2H])([2H])C(c2cc(F)c3c(c2F)C(=O)N([C@@H]2CCC(=O)NC2=O)C3=O)C([2H])([2H])C1([2H])[2H] ; *N1C([2H])([2H])C([2H])([2H])C(c2cc(F)c3c(c2F)C(=O)N([C@H]2CCC(=O)NC2=O)C3=O)C([2H])([2H])C1([2H])[2H] ; *N1C([2H])([2H])C(c2cc(F)c3c(c2F)C(=O)N([C@@H]2CCC(=O)NC2=O)C3=O)C1([2H])[2H] ; *N1C([2H])([2H])C(c2cc(F)c3c(c2F)C(=O)N([C@H]2CCC(=O)NC2=O)C3=O)C1([2H])[2H]]

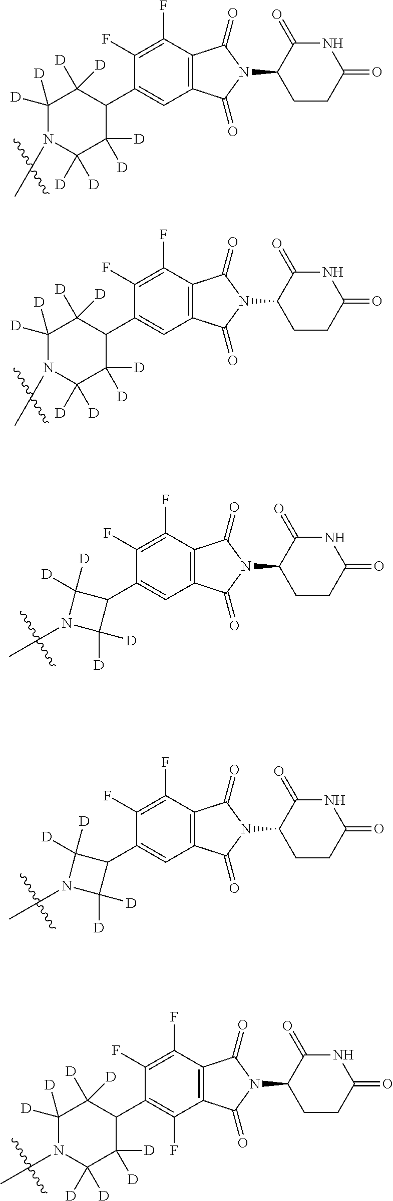 [SMILES: *N1C([2H])([2H])C([2H])([2H])C(c2cc3c(c(F)c2F)C(=O)N([C@@H]2CCC(=O)NC2=O)C3=O)C([2H])([2H])C1([2H])[2H] ; *N1C([2H])([2H])C([2H])([2H])C(c2cc3c(c(F)c2F)C(=O)N([C@H]2CCC(=O)NC2=O)C3=O)C([2H])([2H])C1([2H])[2H] ; *N1C([2H])([2H])C(c2cc3c(c(F)c2F)C(=O)N([C@@H]2CCC(=O)NC2=O)C3=O)C1([2H])[2H] ; *N1C([2H])([2H])C(c2cc3c(c(F)c2F)C(=O)N([C@H]2CCC(=O)NC2=O)C3=O)C1([2H])[2H] ; *N1C([2H])([2H])C([2H])([2H])C(c2c(F)c(F)c3c(c2F)C(=O)N([C@@H]2CCC(=O)NC2=O)C3=O)C([2H])([2H])C1([2H])[2H]]

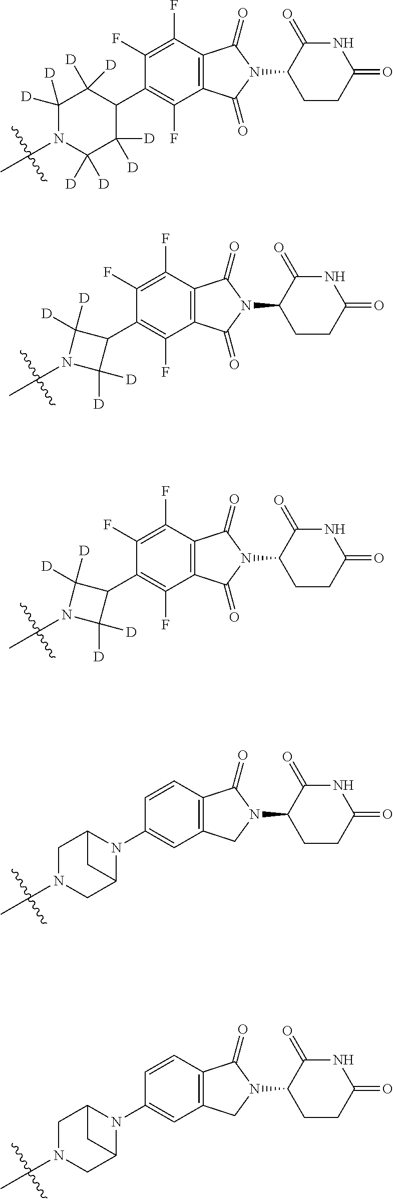 [SMILES: *N1C([2H])([2H])C([2H])([2H])C(c2c(F)c(F)c3c(c2F)C(=O)N([C@H]2CCC(=O)NC2=O)C3=O)C([2H])([2H])C1([2H])[2H] ; *N1C([2H])([2H])C(c2c(F)c(F)c3c(c2F)C(=O)N([C@@H]2CCC(=O)NC2=O)C3=O)C1([2H])[2H] ; *N1C([2H])([2H])C(c2c(F)c(F)c3c(c2F)C(=O)N([C@H]2CCC(=O)NC2=O)C3=O)C1([2H])[2H] ; *N1CC2CC(C1)N2c1ccc2c(c1)CN([C@@H]1CCC(=O)NC1=O)C2=O ; *N1CC2CC(C1)N2c1ccc2c(c1)CN([C@H]1CCC(=O)NC1=O)C2=O]

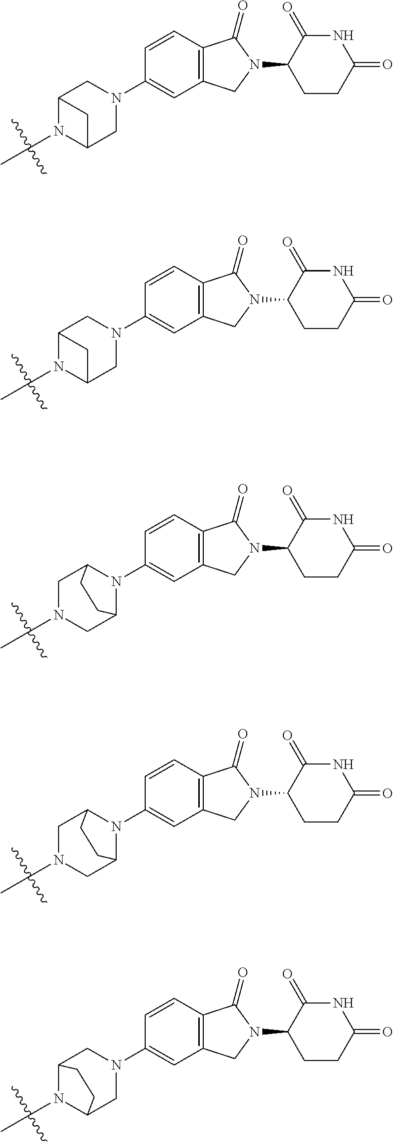 [SMILES: *N1C2CC1CN(c1ccc3c(c1)CN([C@@H]1CCC(=O)NC1=O)C3=O)C2 ; *N1C2CC1CN(c1ccc3c(c1)CN([C@H]1CCC(=O)NC1=O)C3=O)C2 ; *N1CC2CCC(C1)N2c1ccc2c(c1)CN([C@@H]1CCC(=O)NC1=O)C2=O ; *N1CC2CCC(C1)N2c1ccc2c(c1)CN([C@H]1CCC(=O)NC1=O)C2=O ; *N1C2CCC1CN(c1ccc3c(c1)CN([C@@H]1CCC(=O)NC1=O)C3=O)C2]

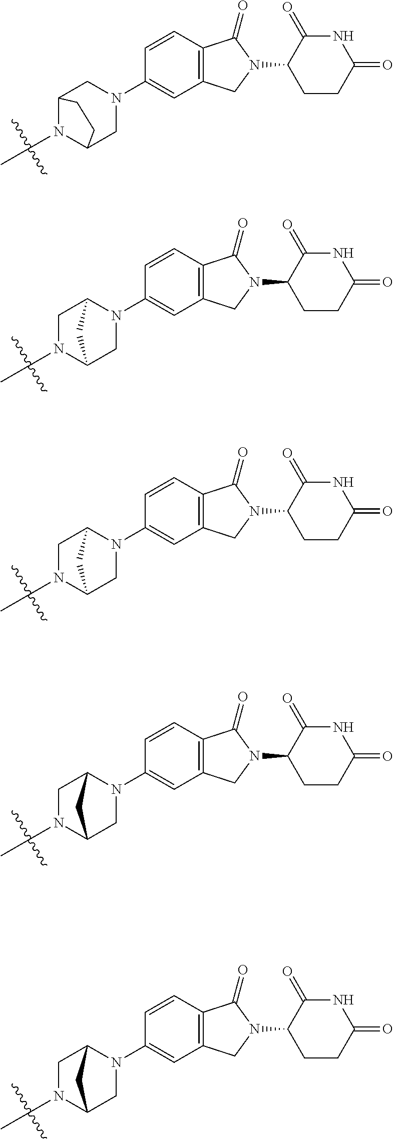 [SMILES: *N1C2CCC1CN(c1ccc3c(c1)CN([C@H]1CCC(=O)NC1=O)C3=O)C2 ; *N1C[C@@H]2C[C@H]1CN2c1ccc2c(c1)CN([C@@H]1CCC(=O)NC1=O)C2=O ; *N1C[C@@H]2C[C@H]1CN2c1ccc2c(c1)CN([C@H]1CCC(=O)NC1=O)C2=O ; *N1C[C@H]2C[C@@H]1CN2c1ccc2c(c1)CN([C@@H]1CCC(=O)NC1=O)C2=O ; *N1C[C@H]2C[C@@H]1CN2c1ccc2c(c1)CN([C@H]1CCC(=O)NC1=O)C2=O]

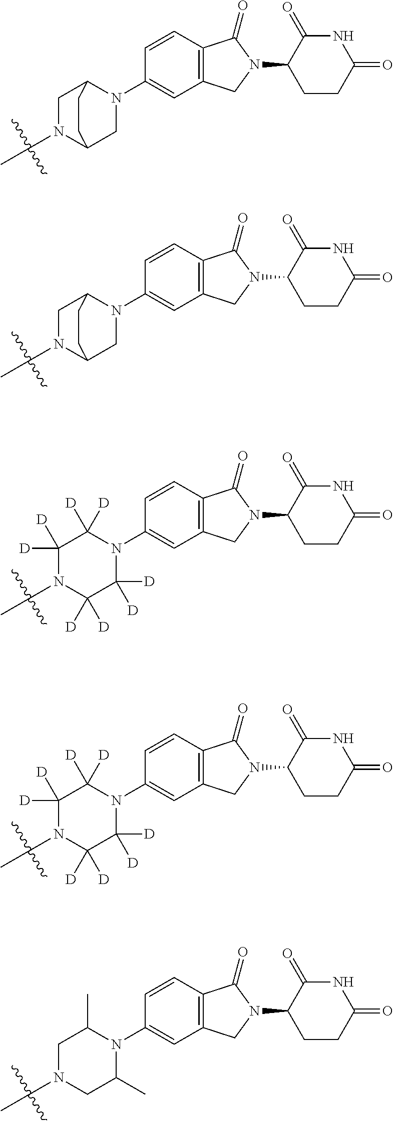 [SMILES: *N1CC2CCC1CN2c1ccc2c(c1)CN([C@@H]1CCC(=O)NC1=O)C2=O ; *N1CC2CCC1CN2c1ccc2c(c1)CN([C@H]1CCC(=O)NC1=O)C2=O ; *N1C([2H])([2H])C([2H])([2H])N(c2ccc3c(c2)CN([C@@H]2CCC(=O)NC2=O)C3=O)C([2H])([2H])C1([2H])[2H] ; *N1C([2H])([2H])C([2H])([2H])N(c2ccc3c(c2)CN([C@H]2CCC(=O)NC2=O)C3=O)C([2H])([2H])C1([2H])[2H] ; *N1CC(C)N(c2ccc3c(c2)CN([C@@H]2CCC(=O)NC2=O)C3=O)C(C)C1]

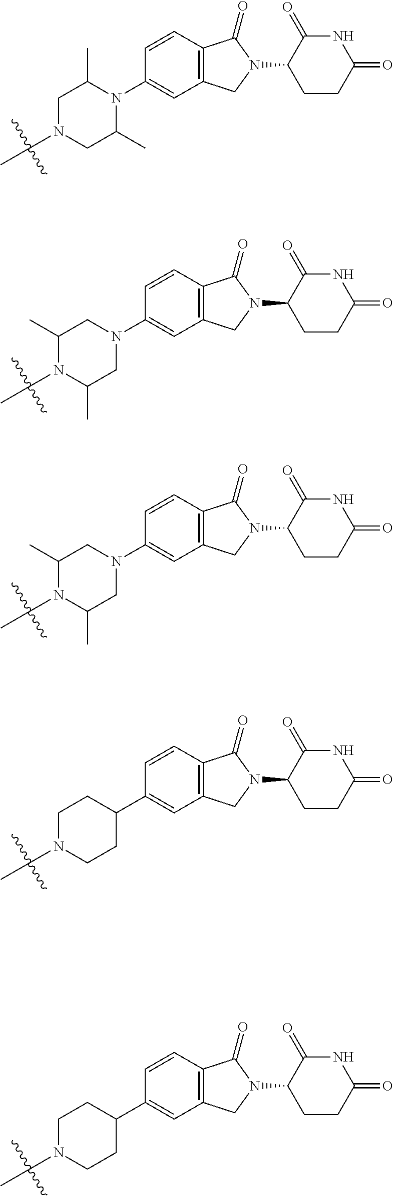 [SMILES: *N1CC(C)N(c2ccc3c(c2)CN([C@H]2CCC(=O)NC2=O)C3=O)C(C)C1 ; *N1C(C)CN(c2ccc3c(c2)CN([C@@H]2CCC(=O)NC2=O)C3=O)CC1C ; *N1C(C)CN(c2ccc3c(c2)CN([C@H]2CCC(=O)NC2=O)C3=O)CC1C ; *N1CCC(c2ccc3c(c2)CN([C@@H]2CCC(=O)NC2=O)C3=O)CC1 ; *N1CCC(c2ccc3c(c2)CN([C@H]2CCC(=O)NC2=O)C3=O)CC1]

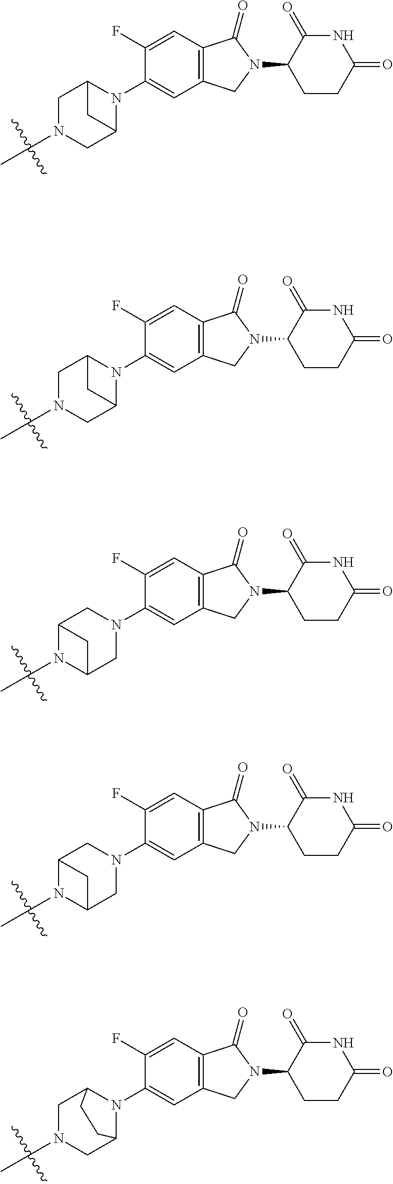 [SMILES: *N1CC2CC(C1)N2c1cc2c(cc1F)C(=O)N([C@@H]1CCC(=O)NC1=O)C2 ; *N1CC2CC(C1)N2c1cc2c(cc1F)C(=O)N([C@H]1CCC(=O)NC1=O)C2 ; *N1C2CC1CN(c1cc3c(cc1F)C(=O)N([C@@H]1CCC(=O)NC1=O)C3)C2 ; *N1C2CC1CN(c1cc3c(cc1F)C(=O)N([C@H]1CCC(=O)NC1=O)C3)C2 ; *N1CC2CCC(C1)N2c1cc2c(cc1F)C(=O)N([C@@H]1CCC(=O)NC1=O)C2]

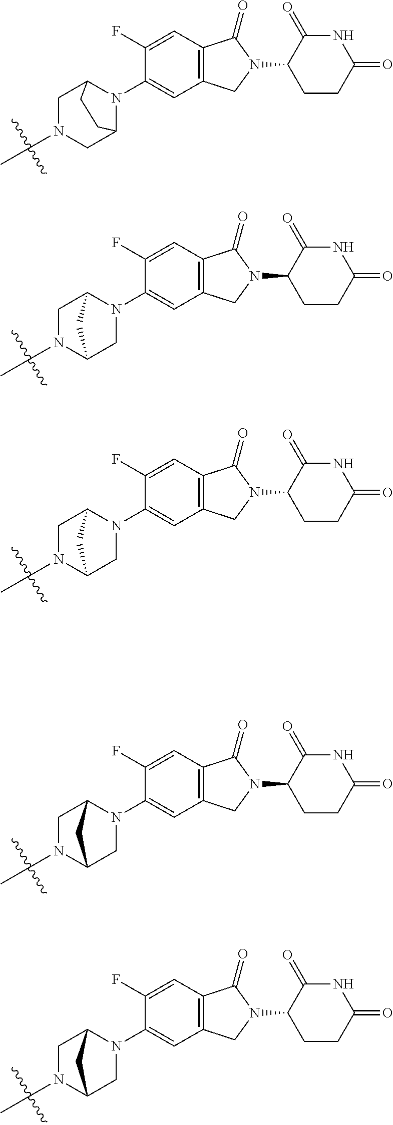 [SMILES: *N1CC2CCC(C1)N2c1cc2c(cc1F)C(=O)N([C@H]1CCC(=O)NC1=O)C2 ; *N1C[C@@H]2C[C@H]1CN2c1cc2c(cc1F)C(=O)N([C@@H]1CCC(=O)NC1=O)C2 ; *N1C[C@@H]2C[C@H]1CN2c1cc2c(cc1F)C(=O)N([C@H]1CCC(=O)NC1=O)C2 ; *N1C[C@H]2C[C@@H]1CN2c1cc2c(cc1F)C(=O)N([C@@H]1CCC(=O)NC1=O)C2 ; *N1C[C@H]2C[C@@H]1CN2c1cc2c(cc1F)C(=O)N([C@H]1CCC(=O)NC1=O)C2]

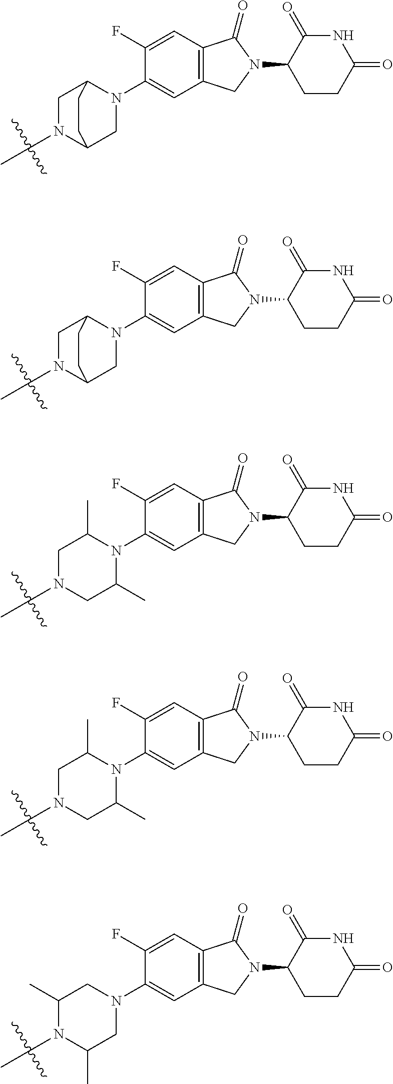 [SMILES: *N1CC2CCC1CN2c1cc2c(cc1F)C(=O)N([C@@H]1CCC(=O)NC1=O)C2 ; *N1CC2CCC1CN2c1cc2c(cc1F)C(=O)N([C@H]1CCC(=O)NC1=O)C2 ; *N1CC(C)N(c2cc3c(cc2F)C(=O)N([C@@H]2CCC(=O)NC2=O)C3)C(C)C1 ; *N1CC(C)N(c2cc3c(cc2F)C(=O)N([C@H]2CCC(=O)NC2=O)C3)C(C)C1 ; *N1C(C)CN(c2cc3c(cc2F)C(=O)N([C@@H]2CCC(=O)NC2=O)C3)CC1C]

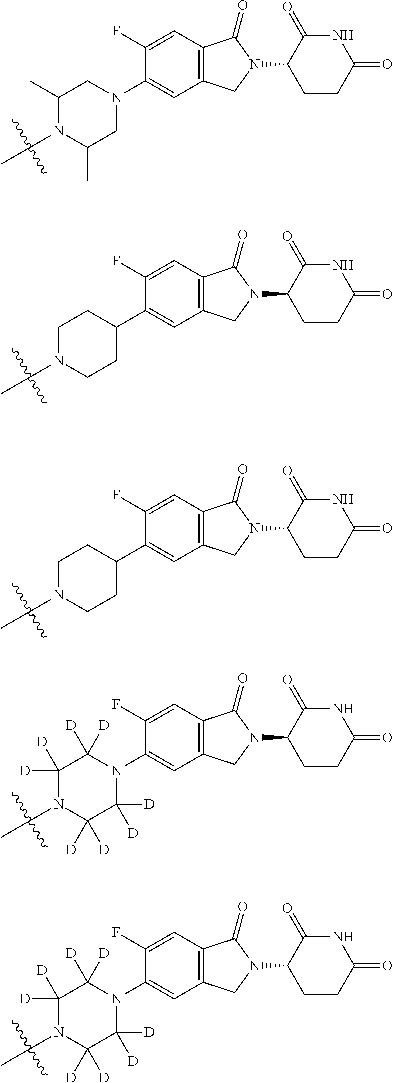 [SMILES: *N1C(C)CN(c2cc3c(cc2F)C(=O)N([C@H]2CCC(=O)NC2=O)C3)CC1C ; *N1CCC(c2cc3c(cc2F)C(=O)N([C@@H]2CCC(=O)NC2=O)C3)CC1 ; *N1CCC(c2cc3c(cc2F)C(=O)N([C@H]2CCC(=O)NC2=O)C3)CC1 ; *N1C([2H])([2H])C([2H])([2H])N(c2cc3c(cc2F)C(=O)N([C@@H]2CCC(=O)NC2=O)C3)C([2H])([2H])C1([2H])[2H] ; *N1C([2H])([2H])C([2H])([2H])N(c2cc3c(cc2F)C(=O)N([C@H]2CCC(=O)NC2=O)C3)C([2H])([2H])C1([2H])[2H]]

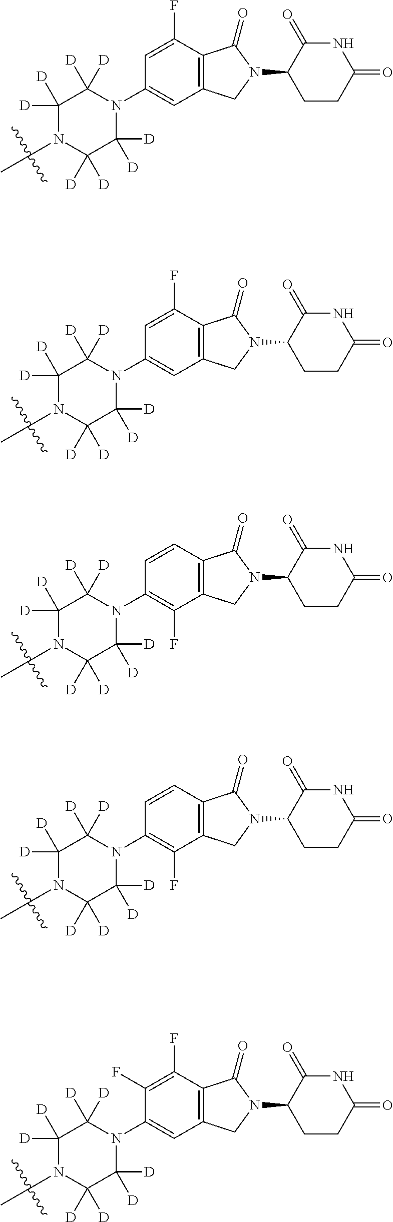 [SMILES: *N1C([2H])([2H])C([2H])([2H])N(c2cc(F)c3c(c2)CN([C@@H]2CCC(=O)NC2=O)C3=O)C([2H])([2H])C1([2H])[2H] ; *N1C([2H])([2H])C([2H])([2H])N(c2cc(F)c3c(c2)CN([C@H]2CCC(=O)NC2=O)C3=O)C([2H])([2H])C1([2H])[2H] ; *N1C([2H])([2H])C([2H])([2H])N(c2ccc3c(c2F)CN([C@@H]2CCC(=O)NC2=O)C3=O)C([2H])([2H])C1([2H])[2H] ; *N1C([2H])([2H])C([2H])([2H])N(c2ccc3c(c2F)CN([C@H]2CCC(=O)NC2=O)C3=O)C([2H])([2H])C1([2H])[2H] ; *N1C([2H])([2H])C([2H])([2H])N(c2cc3c(c(F)c2F)C(=O)N([C@@H]2CCC(=O)NC2=O)C3)C([2H])([2H])C1([2H])[2H]]

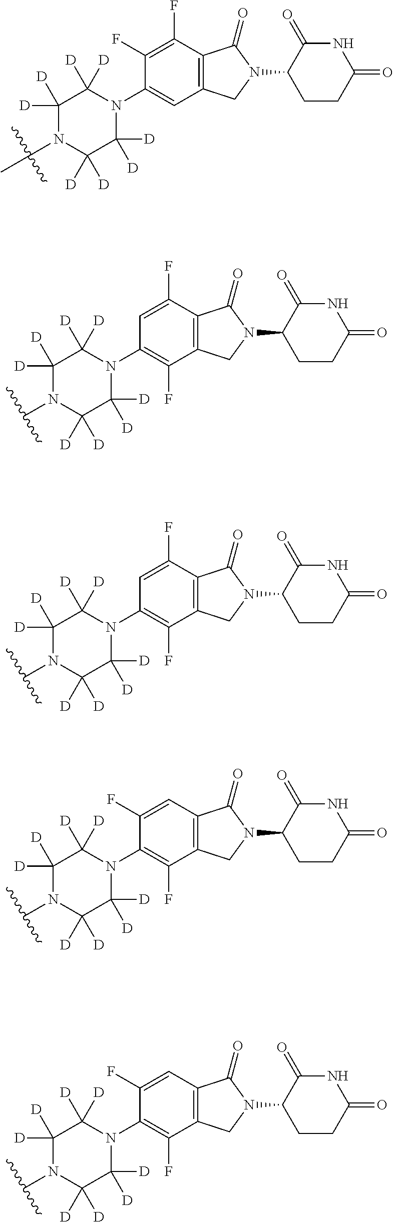

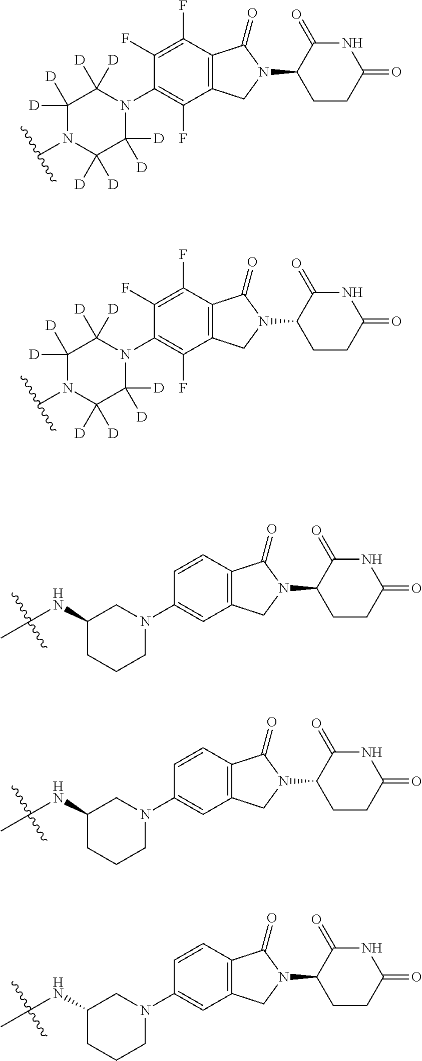 [SMILES: *N1C([2H])([2H])C([2H])([2H])N(c2c(F)c(F)c3c(c2F)CN([C@@H]2CCC(=O)NC2=O)C3=O)C([2H])([2H])C1([2H])[2H] ; *N1C([2H])([2H])C([2H])([2H])N(c2c(F)c(F)c3c(c2F)CN([C@H]2CCC(=O)NC2=O)C3=O)C([2H])([2H])C1([2H])[2H] ; *N[C@@H]1CCCN(c2ccc3c(c2)CN([C@@H]2CCC(=O)NC2=O)C3=O)C1 ; *N[C@@H]1CCCN(c2ccc3c(c2)CN([C@H]2CCC(=O)NC2=O)C3=O)C1 ; *N[C@H]1CCCN(c2ccc3c(c2)CN([C@@H]2CCC(=O)NC2=O)C3=O)C1]

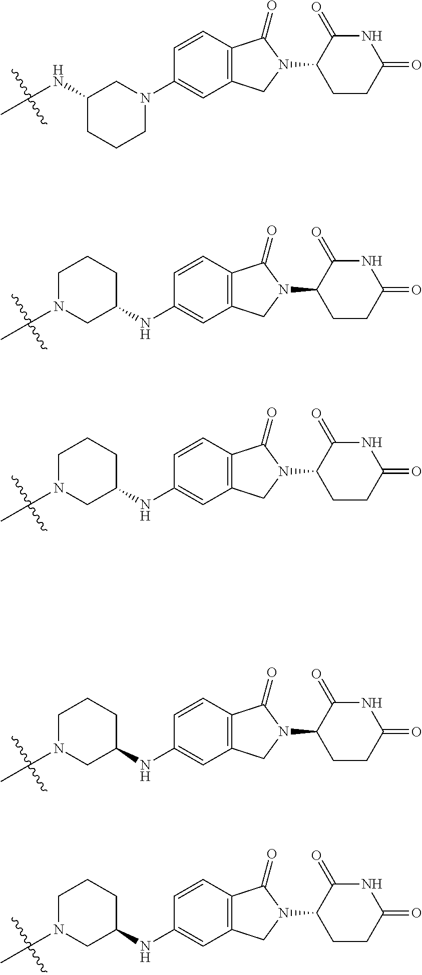 [SMILES: *N[C@H]1CCCN(c2ccc3c(c2)CN([C@H]2CCC(=O)NC2=O)C3=O)C1 ; *N1CCC[C@H](Nc2ccc3c(c2)CN([C@@H]2CCC(=O)NC2=O)C3=O)C1 ; *N1CCC[C@H](Nc2ccc3c(c2)CN([C@H]2CCC(=O)NC2=O)C3=O)C1 ; *N1CCC[C@@H](Nc2ccc3c(c2)CN([C@@H]2CCC(=O)NC2=O)C3=O)C1 ; *N1CCC[C@@H](Nc2ccc3c(c2)CN([C@H]2CCC(=O)NC2=O)C3=O)C1]

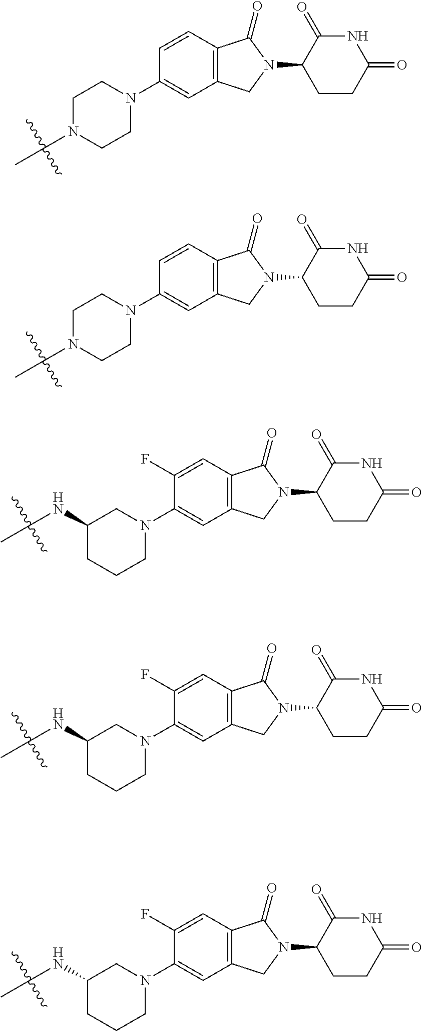 [SMILES: *N1CCN(c2ccc3c(c2)CN([C@@H]2CCC(=O)NC2=O)C3=O)CC1 ; *N1CCN(c2ccc3c(c2)CN([C@H]2CCC(=O)NC2=O)C3=O)CC1 ; *N[C@@H]1CCCN(c2cc3c(cc2F)C(=O)N([C@@H]2CCC(=O)NC2=O)C3)C1 ; *N[C@@H]1CCCN(c2cc3c(cc2F)C(=O)N([C@H]2CCC(=O)NC2=O)C3)C1 ; *N[C@H]1CCCN(c2cc3c(cc2F)C(=O)N([C@@H]2CCC(=O)NC2=O)C3)C1]

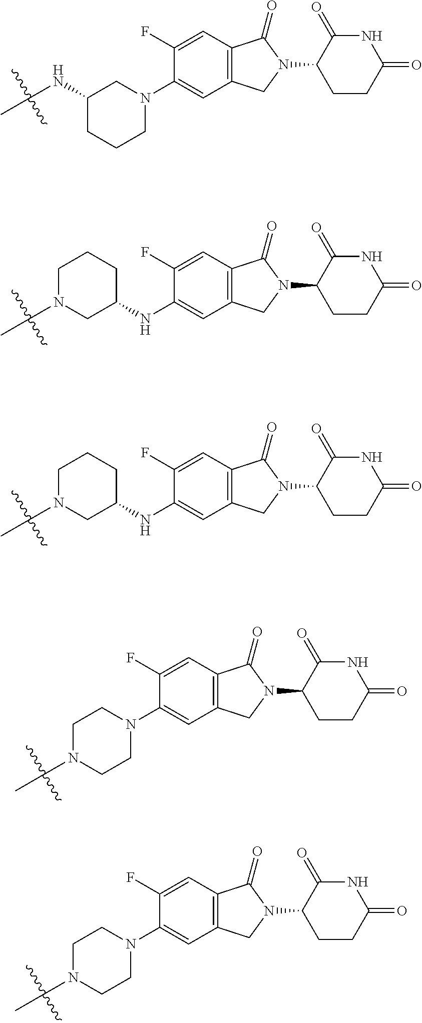 [SMILES: *N[C@H]1CCCN(c2cc3c(cc2F)C(=O)N([C@H]2CCC(=O)NC2=O)C3)C1 ; *N1CCC[C@H](Nc2cc3c(cc2F)C(=O)N([C@@H]2CCC(=O)NC2=O)C3)C1 ; *N1CCC[C@H](Nc2cc3c(cc2F)C(=O)N([C@H]2CCC(=O)NC2=O)C3)C1 ; *N1CCN(c2cc3c(cc2F)C(=O)N([C@@H]2CCC(=O)NC2=O)C3)CC1 ; *N1CCN(c2cc3c(cc2F)C(=O)N([C@H]2CCC(=O)NC2=O)C3)CC1]

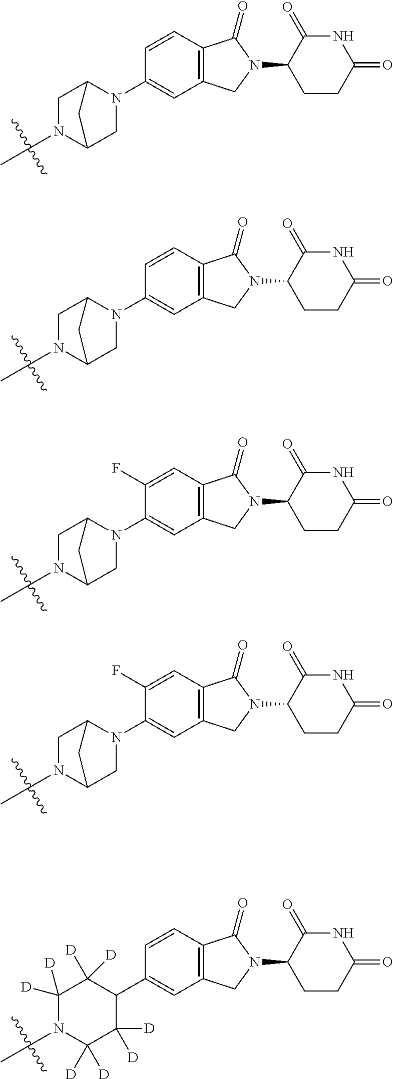 [SMILES: *N1CC2CC1CN2c1ccc2c(c1)CN([C@@H]1CCC(=O)NC1=O)C2=O ; *N1CC2CC1CN2c1ccc2c(c1)CN([C@H]1CCC(=O)NC1=O)C2=O ; *N1CC2CC1CN2c1cc2c(cc1F)C(=O)N([C@@H]1CCC(=O)NC1=O)C2 ; *N1CC2CC1CN2c1cc2c(cc1F)C(=O)N([C@H]1CCC(=O)NC1=O)C2 ; *N1C([2H])([2H])C([2H])([2H])C(c2ccc3c(c2)CN([C@@H]2CCC(=O)NC2=O)C3=O)C([2H])([2H])C1([2H])[2H]]

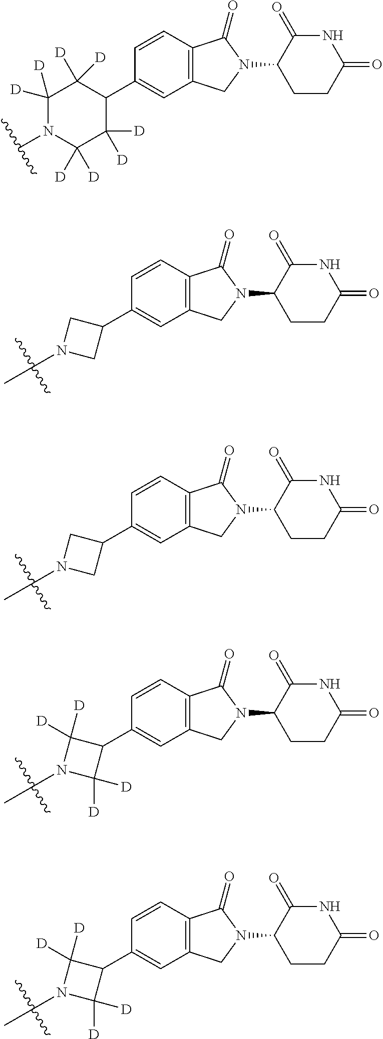 [SMILES: *N1C([2H])([2H])C([2H])([2H])C(c2ccc3c(c2)CN([C@H]2CCC(=O)NC2=O)C3=O)C([2H])([2H])C1([2H])[2H] ; *N1CC(c2ccc3c(c2)CN([C@@H]2CCC(=O)NC2=O)C3=O)C1 ; *N1CC(c2ccc3c(c2)CN([C@H]2CCC(=O)NC2=O)C3=O)C1 ; *N1C([2H])([2H])C(c2ccc3c(c2)CN([C@@H]2CCC(=O)NC2=O)C3=O)C1([2H])[2H] ; *N1C([2H])([2H])C(c2ccc3c(c2)CN([C@H]2CCC(=O)NC2=O)C3=O)C1([2H])[2H]]

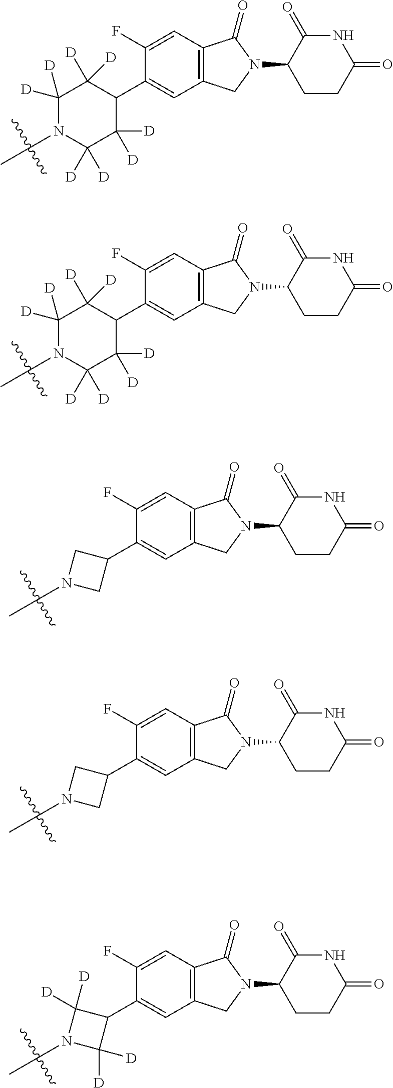 [SMILES: *N1C([2H])([2H])C([2H])([2H])C(c2cc3c(cc2F)C(=O)N([C@@H]2CCC(=O)NC2=O)C3)C([2H])([2H])C1([2H])[2H] ; *N1C([2H])([2H])C([2H])([2H])C(c2cc3c(cc2F)C(=O)N([C@H]2CCC(=O)NC2=O)C3)C([2H])([2H])C1([2H])[2H] ; *N1CC(c2cc3c(cc2F)C(=O)N([C@@H]2CCC(=O)NC2=O)C3)C1 ; *N1CC(c2cc3c(cc2F)C(=O)N([C@H]2CCC(=O)NC2=O)C3)C1 ; *N1C([2H])([2H])C(c2cc3c(cc2F)C(=O)N([C@@H]2CCC(=O)NC2=O)C3)C1([2H])[2H]]

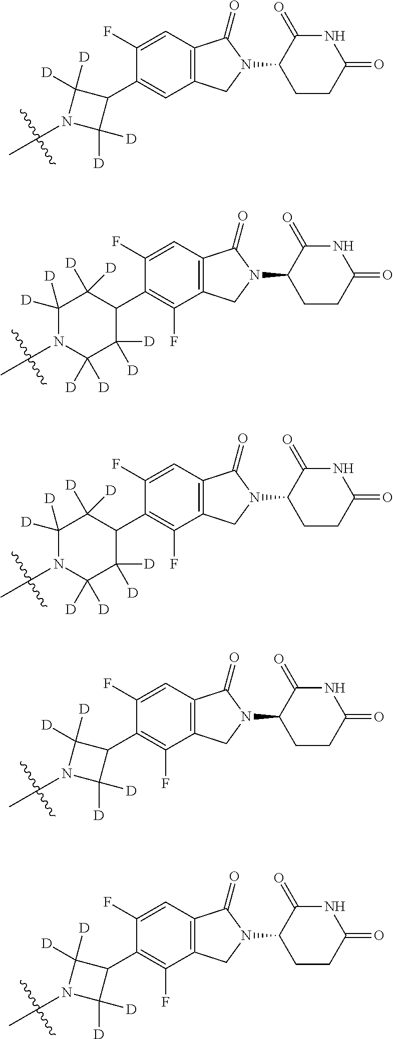 [SMILES: *N1C([2H])([2H])C(c2cc3c(cc2F)C(=O)N([C@H]2CCC(=O)NC2=O)C3)C1([2H])[2H] ; *N1C([2H])([2H])C([2H])([2H])C(c2c(F)cc3c(c2F)CN([C@@H]2CCC(=O)NC2=O)C3=O)C([2H])([2H])C1([2H])[2H] ; *N1C([2H])([2H])C([2H])([2H])C(c2c(F)cc3c(c2F)CN([C@H]2CCC(=O)NC2=O)C3=O)C([2H])([2H])C1([2H])[2H] ; *N1C([2H])([2H])C(c2c(F)cc3c(c2F)CN([C@@H]2CCC(=O)NC2=O)C3=O)C1([2H])[2H] ; *N1C([2H])([2H])C(c2c(F)cc3c(c2F)CN([C@H]2CCC(=O)NC2=O)C3=O)C1([2H])[2H]]

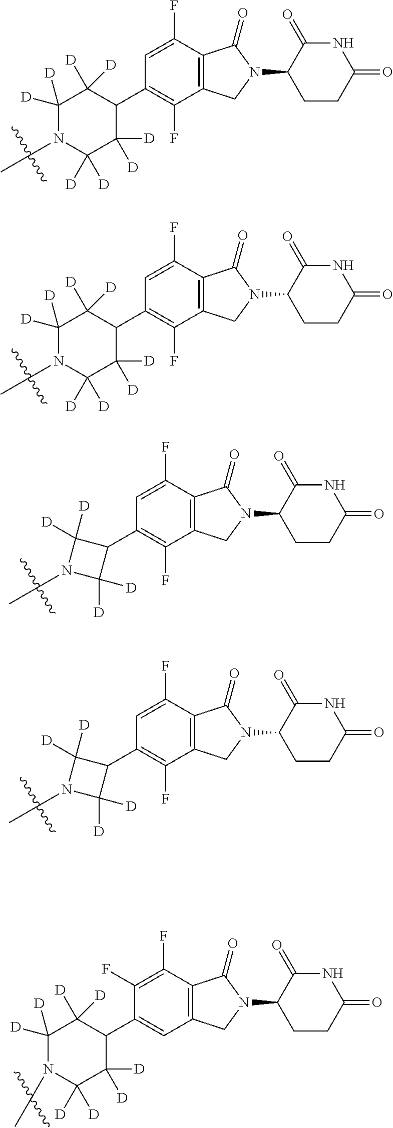 [SMILES: *N1C([2H])([2H])C([2H])([2H])C(c2cc(F)c3c(c2F)CN([C@@H]2CCC(=O)NC2=O)C3=O)C([2H])([2H])C1([2H])[2H] ; *N1C([2H])([2H])C([2H])([2H])C(c2cc(F)c3c(c2F)CN([C@H]2CCC(=O)NC2=O)C3=O)C([2H])([2H])C1([2H])[2H] ; *N1C([2H])([2H])C(c2cc(F)c3c(c2F)CN([C@@H]2CCC(=O)NC2=O)C3=O)C1([2H])[2H] ; *N1C([2H])([2H])C(c2cc(F)c3c(c2F)CN([C@H]2CCC(=O)NC2=O)C3=O)C1([2H])[2H] ; *N1C([2H])([2H])C([2H])([2H])C(c2cc3c(c(F)c2F)C(=O)N([C@@H]2CCC(=O)NC2=O)C3)C([2H])([2H])C1([2H])[2H]]

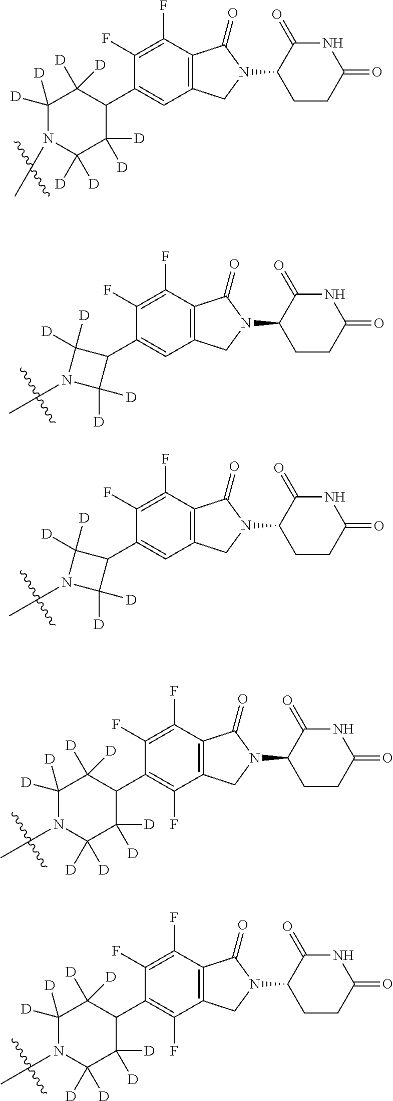 [SMILES: *N1C([2H])([2H])C([2H])([2H])C(c2cc3c(c(F)c2F)C(=O)N([C@H]2CCC(=O)NC2=O)C3)C([2H])([2H])C1([2H])[2H] ; *N1C([2H])([2H])C(c2cc3c(c(F)c2F)C(=O)N([C@@H]2CCC(=O)NC2=O)C3)C1([2H])[2H] ; *N1C([2H])([2H])C(c2cc3c(c(F)c2F)C(=O)N([C@H]2CCC(=O)NC2=O)C3)C1([2H])[2H] ; *N1C([2H])([2H])C([2H])([2H])C(c2c(F)c(F)c3c(c2F)CN([C@@H]2CCC(=O)NC2=O)C3=O)C([2H])([2H])C1([2H])[2H] ; *N1C([2H])([2H])C([2H])([2H])C(c2c(F)c(F)c3c(c2F)CN([C@H]2CCC(=O)NC2=O)C3=O)C([2H])([2H])C1([2H])[2H]]

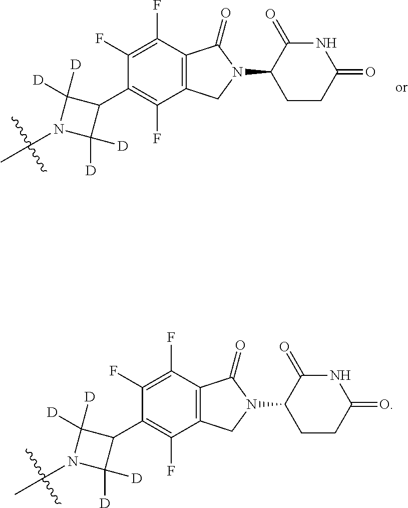 [SMILES: *N1C([2H])([2H])C(c2c(F)c(F)c3c(c2F)CN([C@@H]2CCC(=O)NC2=O)C3=O)C1([2H])[2H] ; *N1C([2H])([2H])C(c2c(F)c(F)c3c(c2F)CN([C@H]2CCC(=O)NC2=O)C3=O)C1([2H])[2H]]

32-34. (canceled)

35. The compound of Formula (III) or salts, enantiomers, stereoisomers, solvates, isotopically enriched analogs, prodrugs, or polymorphs thereof as claimed in claim 20, wherein —W¹-LIN—U₂—R_{c}—U₁— represents the following groups: 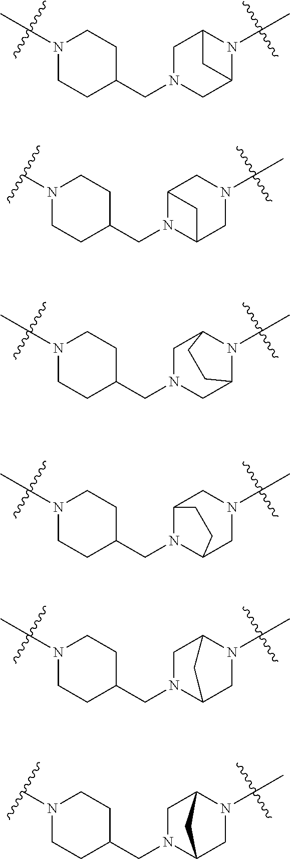 [SMILES: *N1CCC(CN2CC3CC(C2)N3*)CC1 ; *N1CCC(CN2C3CC2CN(*)C3)CC1 ; *N1CCC(CN2CC3CCC(C2)N3*)CC1 ; *N1CCC(CN2C3CCC2CN(*)C3)CC1 ; *N1CCC(CN2CC3CC2CN3*)CC1 ; *N1CCC(CN2C[C@H]3C[C@@H]2CN3*)CC1] 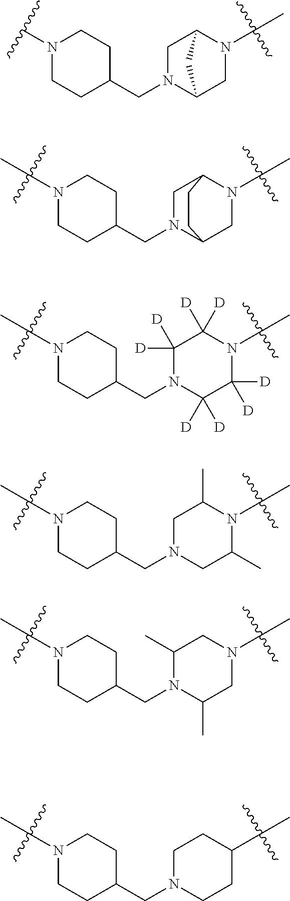 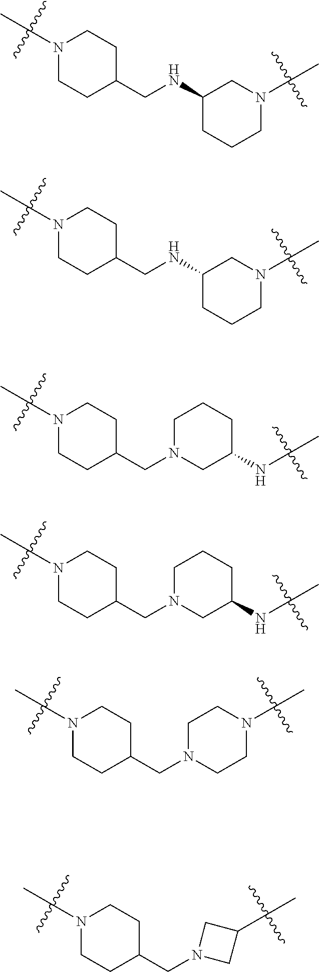 [SMILES: *N1CCC(CN[C@@H]2CCCN(*)C2)CC1 ; *N1CCC(CN[C@H]2CCCN(*)C2)CC1 ; *N[C@H]1CCCN(CC2CCN(*)CC2)C1 ; *N[C@@H]1CCCN(CC2CCN(*)CC2)C1 ; *N1CCC(CN2CCN(*)CC2)CC1 ; *C1CN(CC2CCN(*)CC2)C1] 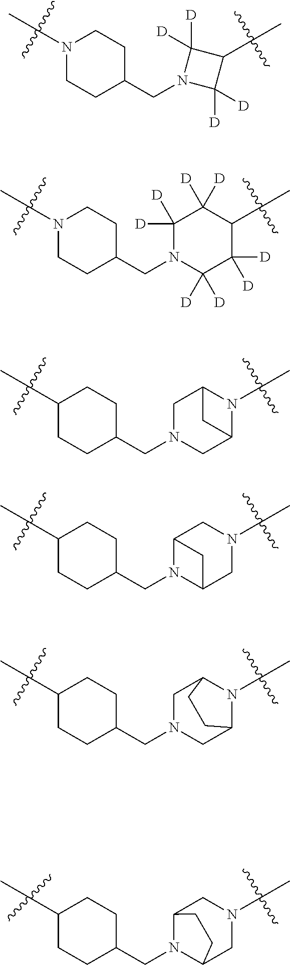 [SMILES: *C1C([2H])([2H])N(CC2CCN(*)CC2)C1([2H])[2H] ; *C1C([2H])([2H])C([2H])([2H])N(CC2CCN(*)CC2)C([2H])([2H])C1([2H])[2H] ; *C1CCC(CN2CC3CC(C2)N3*)CC1 ; *C1CCC(CN2C3CC2CN(*)C3)CC1 ; *C1CCC(CN2CC3CCC(C2)N3*)CC1 ; *C1CCC(CN2C3CCC2CN(*)C3)CC1] 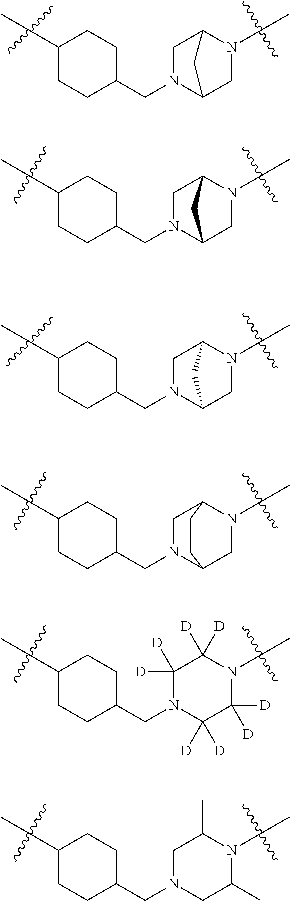 [SMILES: *C1CCC(CN2CC3CC2CN3*)CC1 ; *C1CCC(CN2C[C@H]3C[C@@H]2CN3*)CC1 ; *C1CCC(CN2C[C@@H]3C[C@H]2CN3*)CC1 ; *C1CCC(CN2CC3CCC2CN3*)CC1 ; *C1CCC(CN2C([2H])([2H])C([2H])([2H])N(*)C([2H])([2H])C2([2H])[2H])CC1 ; *C1CCC(CN2CC(C)N(*)C(C)C2)CC1] 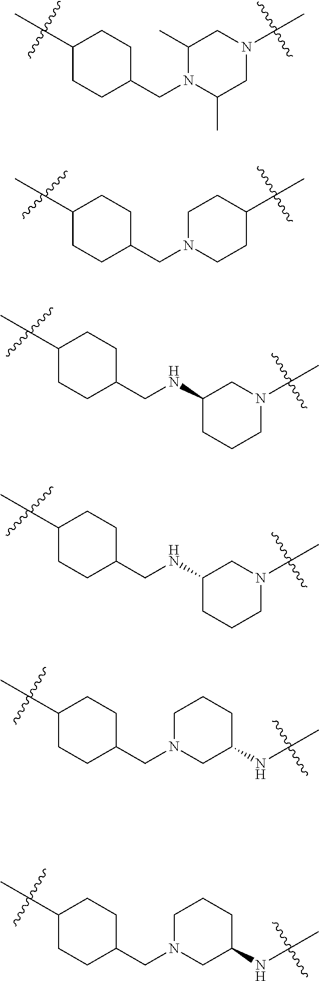 [SMILES: *C1CCC(CN2C(C)CN(*)CC2C)CC1 ; *C1CCC(CN2CCC(*)CC2)CC1 ; *C1CCC(CN[C@@H]2CCCN(*)C2)CC1 ; *C1CCC(CN[C@H]2CCCN(*)C2)CC1 ; *N[C@H]1CCCN(CC2CCC(*)CC2)C1 ; *N[C@@H]1CCCN(CC2CCC(*)CC2)C1] 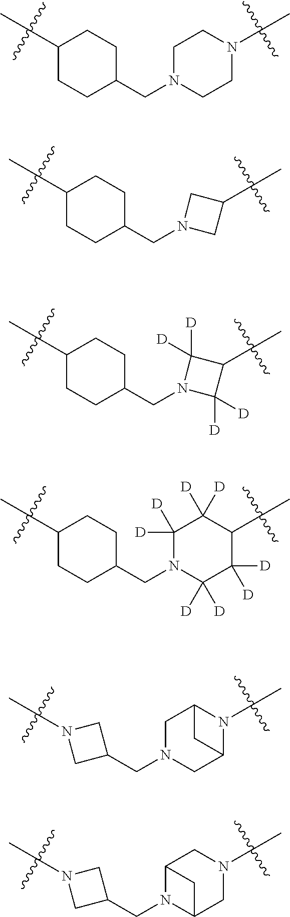 [SMILES: *C1CCC(CN2CCN(*)CC2)CC1 ; *C1CCC(CN2CC(*)C2)CC1 ; *C1CCC(CN2C([2H])([2H])C(*)C2([2H])[2H])CC1 ; *C1CCC(CN2C([2H])([2H])C([2H])([2H])C(*)C([2H])([2H])C2([2H])[2H])CC1 ; *N1CC(CN2CC3CC(C2)N3*)C1 ; *N1CC(CN2C3CC2CN(*)C3)C1] 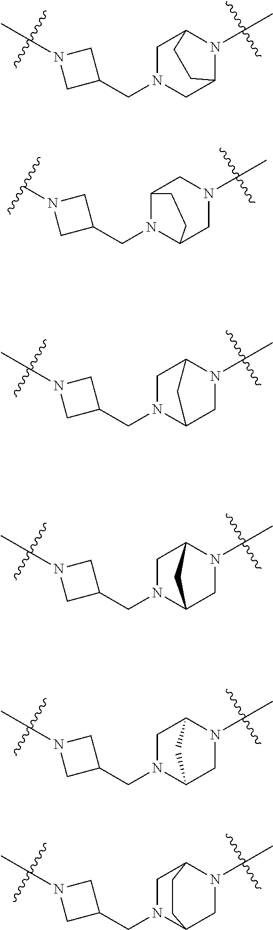 [SMILES: *N1CC(CN2CC3CCC(C2)N3*)C1 ; *N1CC(CN2C3CCC2CN(*)C3)C1 ; *N1CC(CN2CC3CC2CN3*)C1 ; *N1CC(CN2C[C@H]3C[C@@H]2CN3*)C1 ; *N1CC(CN2C[C@@H]3C[C@H]2CN3*)C1 ; *N1CC(CN2CC3CCC2CN3*)C1] 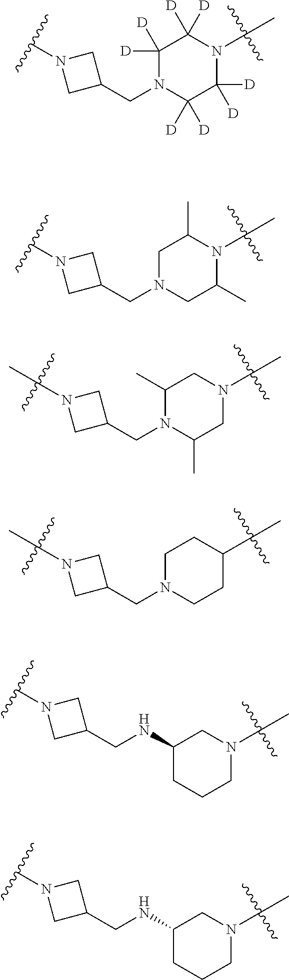 [SMILES: *N1CC(CN2C([2H])([2H])C([2H])([2H])N(*)C([2H])([2H])C2([2H])[2H])C1 ; *N1CC(CN2CC(C)N(*)C(C)C2)C1 ; *N1CC(CN2C(C)CN(*)CC2C)C1 ; *C1CCN(CC2CN(*)C2)CC1 ; *N1CC(CN[C@@H]2CCCN(*)C2)C1 ; *N1CC(CN[C@H]2CCCN(*)C2)C1] 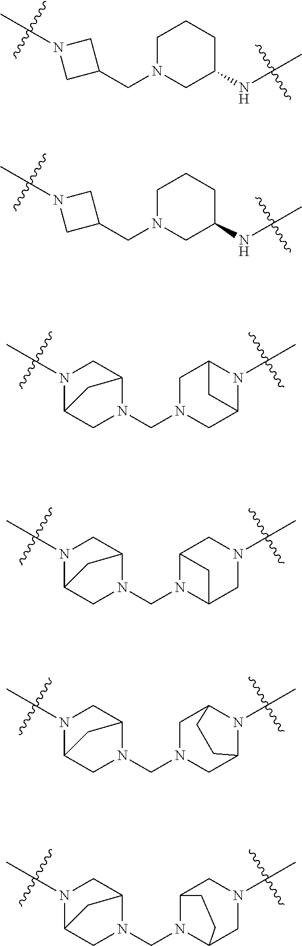 [SMILES: *N[C@H]1CCCN(CC2CN(*)C2)C1 ; *N[C@@H]1CCCN(CC2CN(*)C2)C1 ; *N1CC2CC1CN2CN1CC2CC(C1)N2* ; *N1CC2CC(C1)N2CN1CC2CC1CN2* ; *N1CC2CC1CN2CN1CC2CCC(C1)N2* ; *N1CC2CCC(C1)N2CN1CC2CC1CN2*] 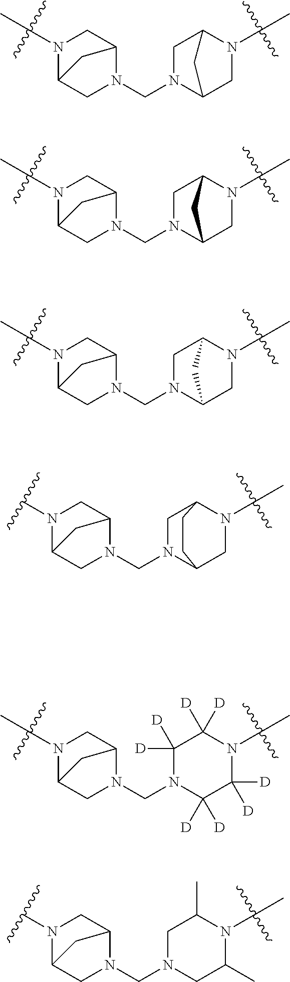 [SMILES: *N1CC2CC1CN2CN1CC2CC1CN2* ; *N1CC2CC1CN2CN1C[C@H]2C[C@@H]1CN2* ; *N1CC2CC1CN2CN1C[C@@H]2C[C@H]1CN2* ; *N1CC2CCC1CN2CN1CC2CC1CN2* ; *N1CC2CC1CN2CN1C([2H])([2H])C([2H])([2H])N(*)C([2H])([2H])C1([2H])[2H] ; *N1CC2CC1CN2CN1CC(C)N(*)C(C)C1] 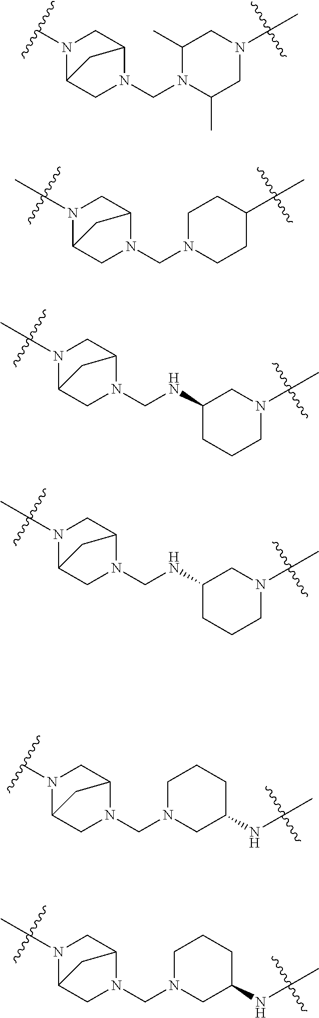 [SMILES: *N1CC(C)N(CN2CC3CC2CN3*)C(C)C1 ; *C1CCN(CN2CC3CC2CN3*)CC1 ; *N1CCC[C@@H](NCN2CC3CC2CN3*)C1 ; *N1CCC[C@H](NCN2CC3CC2CN3*)C1 ; *N[C@H]1CCCN(CN2CC3CC2CN3*)C1 ; *N[C@@H]1CCCN(CN2CC3CC2CN3*)C1] 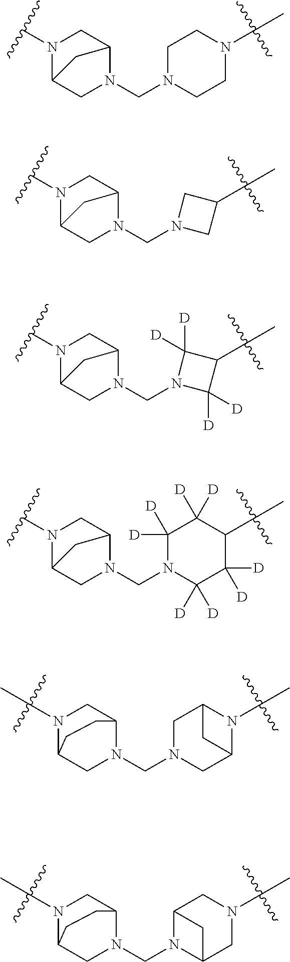 [SMILES: *N1CCN(CN2CC3CC2CN3*)CC1 ; *C1CN(CN2CC3CC2CN3*)C1 ; *C1C([2H])([2H])N(CN2CC3CC2CN3*)C1([2H])[2H] ; *C1C([2H])([2H])C([2H])([2H])N(CN2CC3CC2CN3*)C([2H])([2H])C1([2H])[2H] ; *N1CC2CCC1CN2CN1CC2CC(C1)N2* ; *N1CC2CC(C1)N2CN1CC2CCC1CN2*] 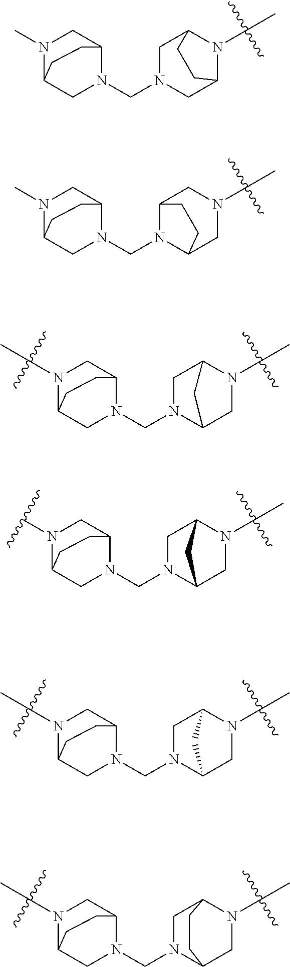 [SMILES: *N1C2CCC1CN(CN1CC3CCC1CN3C)C2 ; *N1CC2CCC(C1)N2CN1CC2CCC1CN2C ; *N1CC2CCC1CN2CN1CC2CC1CN2* ; *N1CC2CCC1CN2CN1C[C@H]2C[C@@H]1CN2* ; *N1CC2CCC1CN2CN1C[C@@H]2C[C@H]1CN2* ; *N1CC2CCC1CN2CN1CC2CCC1CN2*] 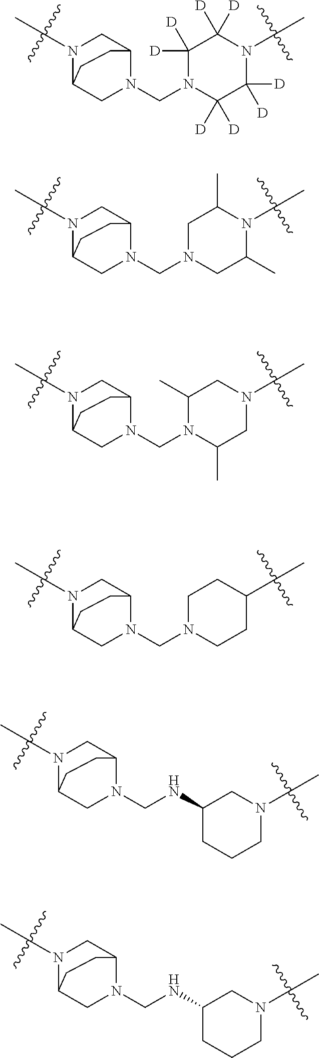 [SMILES: *N1CC2CCC1CN2CN1C([2H])([2H])C([2H])([2H])N(*)C([2H])([2H])C1([2H])[2H] ; *N1CC2CCC1CN2CN1CC(C)N(*)C(C)C1 ; *N1CC(C)N(CN2CC3CCC2CN3*)C(C)C1 ; *C1CCN(CN2CC3CCC2CN3*)CC1 ; *N1CCC[C@@H](NCN2CC3CCC2CN3*)C1 ; *N1CCC[C@H](NCN2CC3CCC2CN3*)C1] 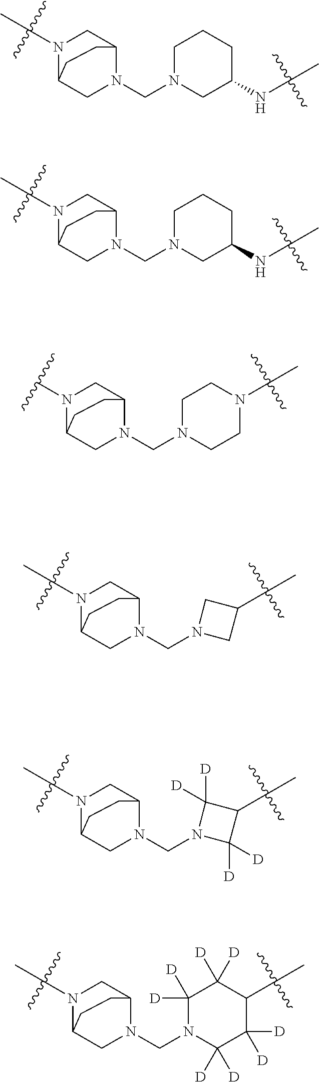 [SMILES: *N[C@H]1CCCN(CN2CC3CCC2CN3*)C1 ; *N[C@@H]1CCCN(CN2CC3CCC2CN3*)C1 ; *N1CCN(CN2CC3CCC2CN3*)CC1 ; *C1CN(CN2CC3CCC2CN3*)C1 ; *C1C([2H])([2H])N(CN2CC3CCC2CN3*)C1([2H])[2H] ; *C1C([2H])([2H])C([2H])([2H])N(CN2CC3CCC2CN3*)C([2H])([2H])C1([2H])[2H]] 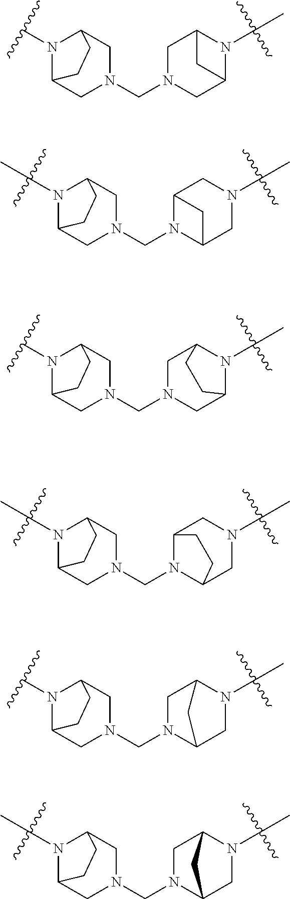 [SMILES: *N1C2CCC1CN(CN1CC3CC(C1)N3*)C2 ; *N1CC2CC(C1)N2CN1CC2CCC(C1)N2* ; *N1C2CCC1CN(CN1CC3CCC(C1)N3*)C2 ; *N1CC2CCC(C1)N2CN1CC2CCC(C1)N2* ; *N1CC2CC1CN2CN1CC2CCC(C1)N2* ; *N1C2CCC1CN(CN1C[C@H]3C[C@@H]1CN3*)C2] 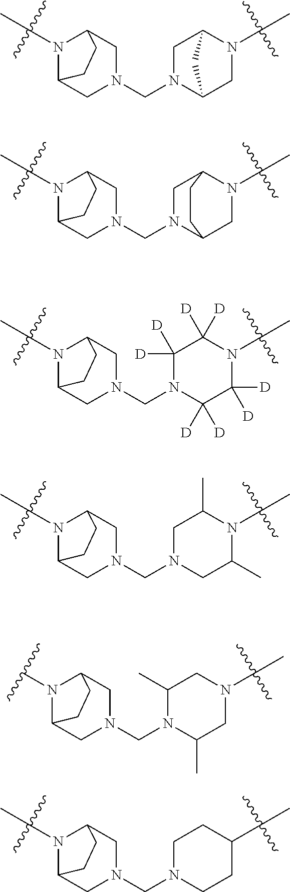 [SMILES: *N1C2CCC1CN(CN1C[C@@H]3C[C@H]1CN3*)C2 ; *N1CC2CCC1CN2CN1CC2CCC(C1)N2* ; *N1C2CCC1CN(CN1C([2H])([2H])C([2H])([2H])N(*)C([2H])([2H])C1([2H])[2H])C2 ; *N1C(C)CN(CN2CC3CCC(C2)N3*)CC1C ; *N1CC(C)N(CN2CC3CCC(C2)N3*)C(C)C1 ; *C1CCN(CN2CC3CCC(C2)N3*)CC1] 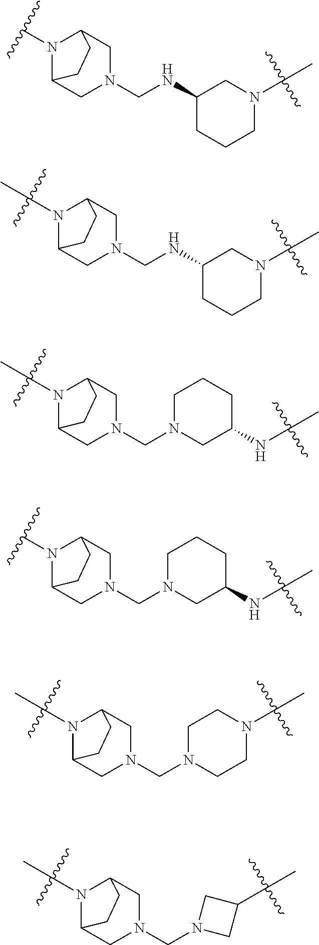 [SMILES: *N1CCC[C@@H](NCN2CC3CCC(C2)N3*)C1 ; *N1CCC[C@H](NCN2CC3CCC(C2)N3*)C1 ; *N[C@H]1CCCN(CN2CC3CCC(C2)N3*)C1 ; *N[C@@H]1CCCN(CN2CC3CCC(C2)N3*)C1 ; *N1CCN(CN2CC3CCC(C2)N3*)CC1 ; *C1CN(CN2CC3CCC(C2)N3*)C1] 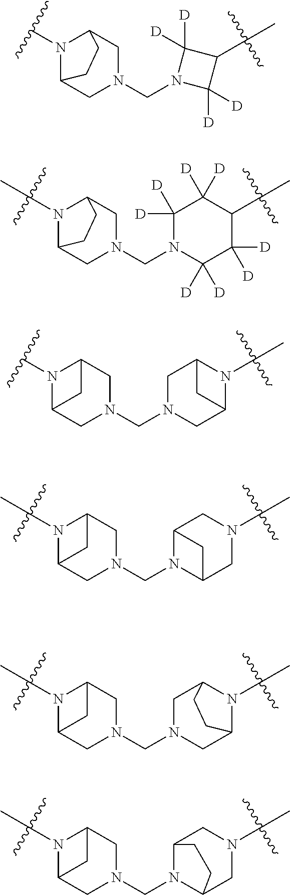 [SMILES: *C1C([2H])([2H])N(CN2CC3CCC(C2)N3*)C1([2H])[2H] ; *C1C([2H])([2H])C([2H])([2H])N(CN2CC3CCC(C2)N3*)C([2H])([2H])C1([2H])[2H] ; *N1C2CC1CN(CN1CC3CC(C1)N3*)C2 ; *N1CC2CC(C1)N2CN1CC2CC(C1)N2* ; *N1C2CCC1CN(CN1CC3CC(C1)N3*)C2 ; *N1CC2CCC(C1)N2CN1CC2CC(C1)N2*] 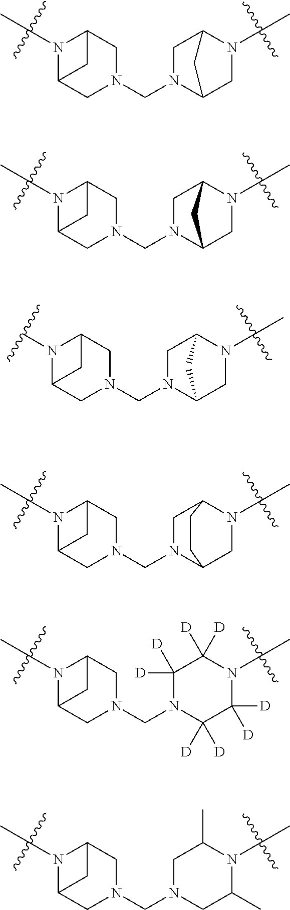 [SMILES: *N1CC2CC1CN2CN1CC2CC(C1)N2* ; *N1C2CC1CN(CN1C[C@H]3C[C@@H]1CN3*)C2 ; *N1C2CC1CN(CN1C[C@@H]3C[C@H]1CN3*)C2 ; *N1CC2CCC1CN2CN1CC2CC(C1)N2* ; *N1C2CC1CN(CN1C([2H])([2H])C([2H])([2H])N(*)C([2H])([2H])C1([2H])[2H])C2 ; *N1C(C)CN(CN2CC3CC(C2)N3*)CC1C] 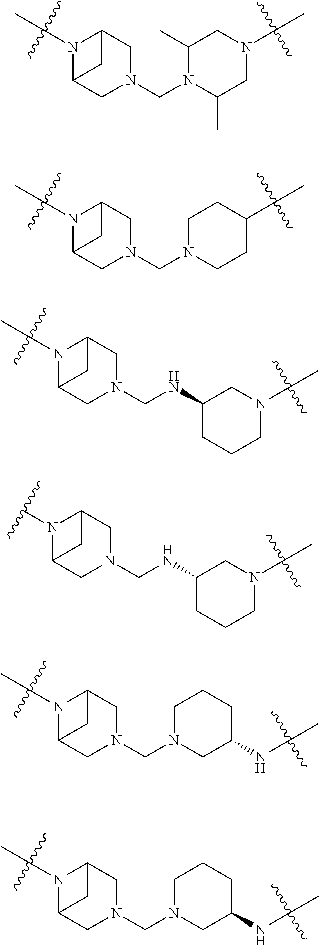 [SMILES: *N1CC(C)N(CN2CC3CC(C2)N3*)C(C)C1 ; *C1CCN(CN2CC3CC(C2)N3*)CC1 ; *N1CCC[C@@H](NCN2CC3CC(C2)N3*)C1 ; *N1CCC[C@H](NCN2CC3CC(C2)N3*)C1 ; *N[C@H]1CCCN(CN2CC3CC(C2)N3*)C1 ; *N[C@@H]1CCCN(CN2CC3CC(C2)N3*)C1] 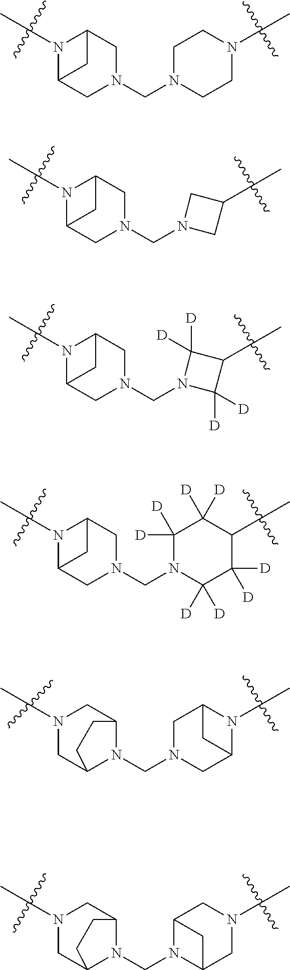 [SMILES: *N1CCN(CN2CC3CC(C2)N3*)CC1 ; *C1CN(CN2CC3CC(C2)N3*)C1 ; *C1C([2H])([2H])N(CN2CC3CC(C2)N3*)C1([2H])[2H] ; *C1C([2H])([2H])C([2H])([2H])N(CN2CC3CC(C2)N3*)C([2H])([2H])C1([2H])[2H] ; *N1CC2CCC(C1)N2CN1CC2CC(C1)N2* ; *N1CC2CCC(C1)N2CN1C2CC1CN(*)C2] 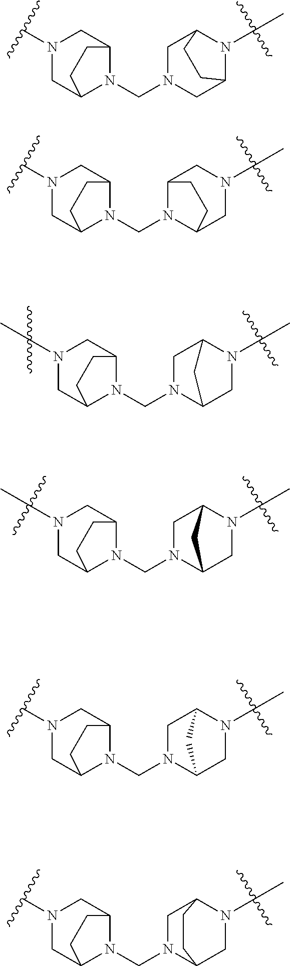 [SMILES: *N1CC2CCC(C1)N2CN1CC2CCC(C1)N2* ; *N1CC2CCC(C1)N2CN1C2CCC1CN(*)C2 ; *N1CC2CCC(C1)N2CN1CC2CC1CN2* ; *N1CC2CCC(C1)N2CN1C[C@H]2C[C@@H]1CN2* ; *N1CC2CCC(C1)N2CN1C[C@@H]2C[C@H]1CN2* ; *N1CC2CCC(C1)N2CN1CC2CCC1CN2*] 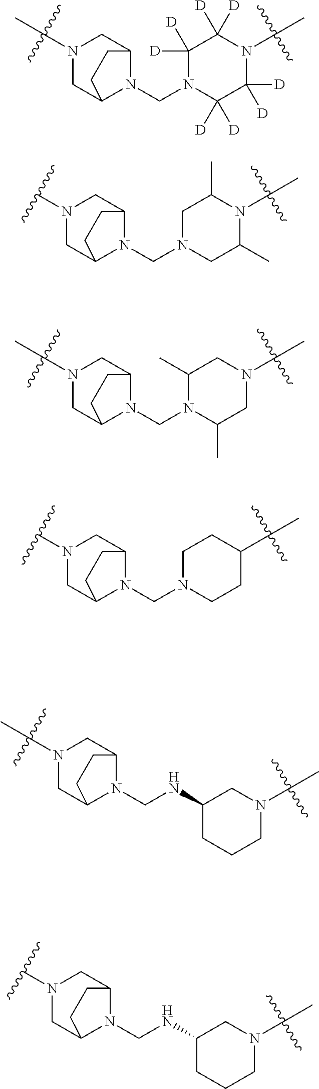 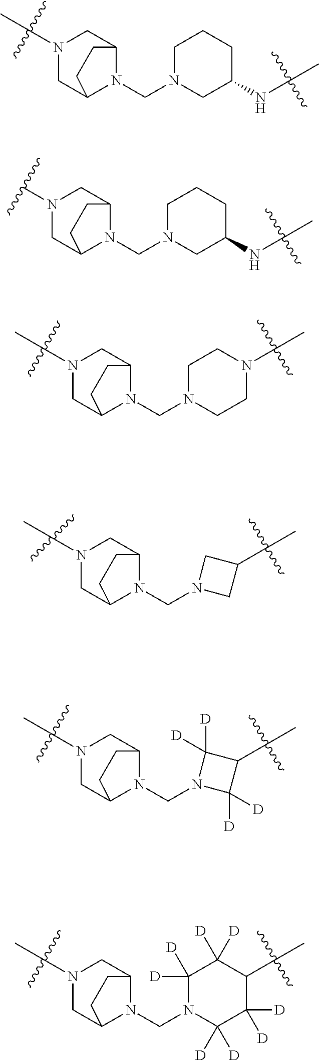 [SMILES: *N[C@H]1CCCN(CN2C3CCC2CN(*)C3)C1 ; *N[C@@H]1CCCN(CN2C3CCC2CN(*)C3)C1 ; *N1CCN(CN2C3CCC2CN(*)C3)CC1 ; *C1CN(CN2C3CCC2CN(*)C3)C1 ; *C1C([2H])([2H])N(CN2C3CCC2CN(*)C3)C1([2H])[2H] ; *C1C([2H])([2H])C([2H])([2H])N(CN2C3CCC2CN(*)C3)C([2H])([2H])C1([2H])[2H]] 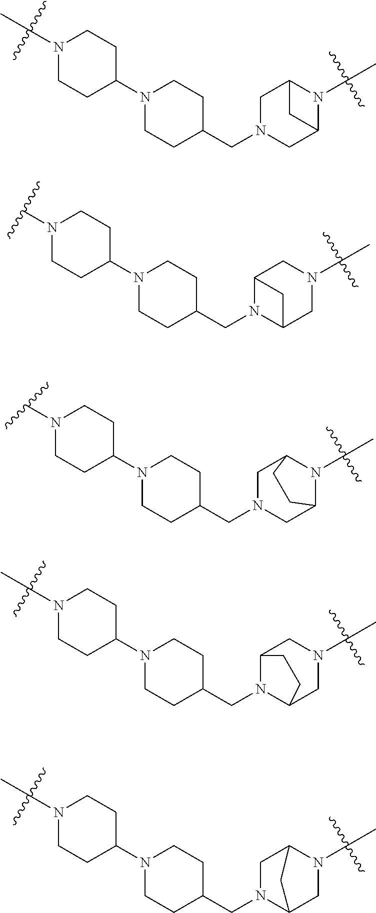 [SMILES: *N1CCC(N2CCC(CN3CC4CC(C3)N4*)CC2)CC1 ; *N1CCC(N2CCC(CN3C4CC3CN(*)C4)CC2)CC1 ; *N1CCC(N2CCC(CN3CC4CCC(C3)N4*)CC2)CC1 ; *N1CCC(N2CCC(CN3C4CCC3CN(*)C4)CC2)CC1 ; *N1CCC(N2CCC(CN3CC4CC3CN4*)CC2)CC1] 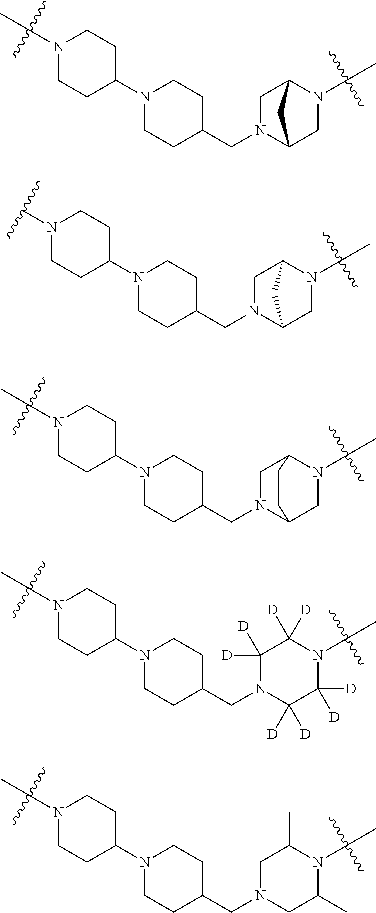 [SMILES: *N1CCC(N2CCC(CN3C[C@H]4C[C@@H]3CN4*)CC2)CC1 ; *N1CCC(N2CCC(CN3C[C@@H]4C[C@H]3CN4*)CC2)CC1 ; *N1CCC(N2CCC(CN3CC4CCC3CN4*)CC2)CC1 ; *N1CCC(N2CCC(CN3C([2H])([2H])C([2H])([2H])N(*)C([2H])([2H])C3([2H])[2H])CC2)CC1 ; *N1CCC(N2CCC(CN3CC(C)N(*)C(C)C3)CC2)CC1] 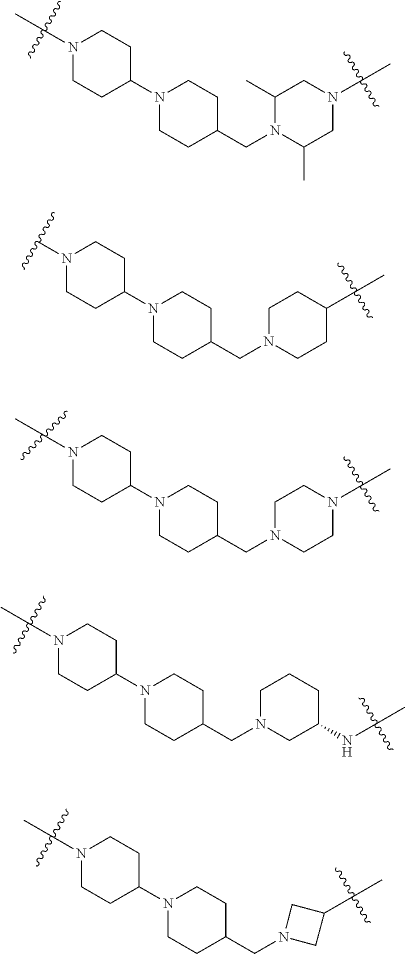 [SMILES: *N1CCC(N2CCC(CN3C(C)CN(*)CC3C)CC2)CC1 ; *C1CCN(CC2CCN(C3CCN(*)CC3)CC2)CC1 ; *N1CCC(N2CCC(CN3CCN(*)CC3)CC2)CC1 ; *N[C@H]1CCCN(CC2CCN(C3CCN(*)CC3)CC2)C1 ; *C1CN(CC2CCN(C3CCN(*)CC3)CC2)C1] 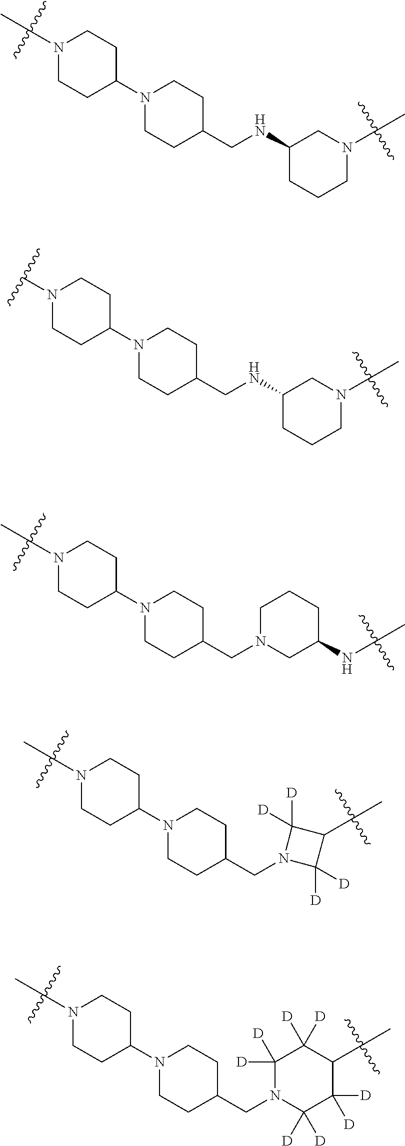 [SMILES: *N1CCC(N2CCC(CN[C@@H]3CCCN(*)C3)CC2)CC1 ; *N1CCC(N2CCC(CN[C@H]3CCCN(*)C3)CC2)CC1 ; *N[C@@H]1CCCN(CC2CCN(C3CCN(*)CC3)CC2)C1 ; *C1C([2H])([2H])N(CC2CCN(C3CCN(*)CC3)CC2)C1([2H])[2H] ; *C1C([2H])([2H])C([2H])([2H])N(CC2CCN(C3CCN(*)CC3)CC2)C([2H])([2H])C1([2H])[2H]] 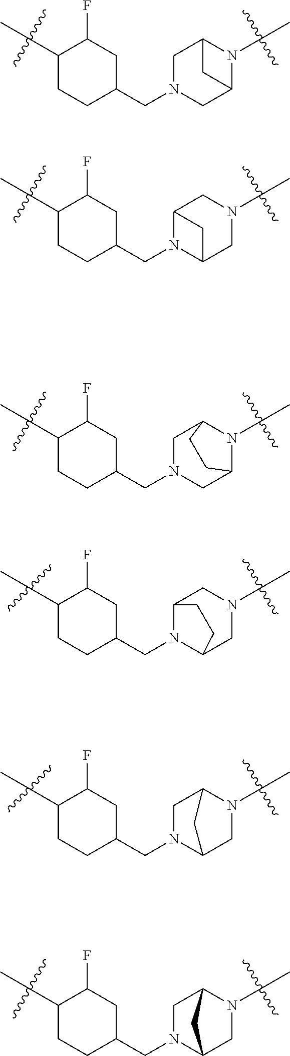 [SMILES: *C1CCC(CN2CC3CC(C2)N3*)CC1F ; *C1CCC(CN2C3CC2CN(*)C3)CC1F ; *C1CCC(CN2CC3CCC(C2)N3*)CC1F ; *C1CCC(CN2C3CCC2CN(*)C3)CC1F ; *C1CCC(CN2CC3CC2CN3*)CC1F ; *C1CCC(CN2C[C@H]3C[C@@H]2CN3*)CC1F] 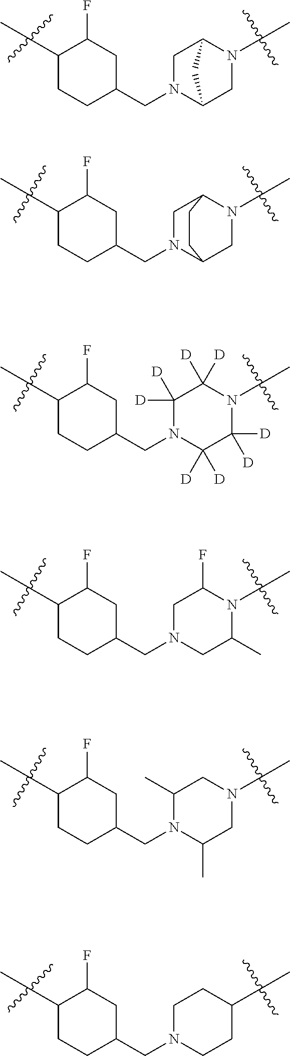 [SMILES: *C1CCC(CN2C[C@@H]3C[C@H]2CN3*)CC1F ; *C1CCC(CN2CC3CCC2CN3*)CC1F ; *C1CCC(CN2C([2H])([2H])C([2H])([2H])N(*)C([2H])([2H])C2([2H])[2H])CC1F ; *C1CCC(CN2CC(C)N(*)C(F)C2)CC1F ; *C1CCC(CN2C(C)CN(*)CC2C)CC1F ; *C1CCN(CC2CCC(*)C(F)C2)CC1] 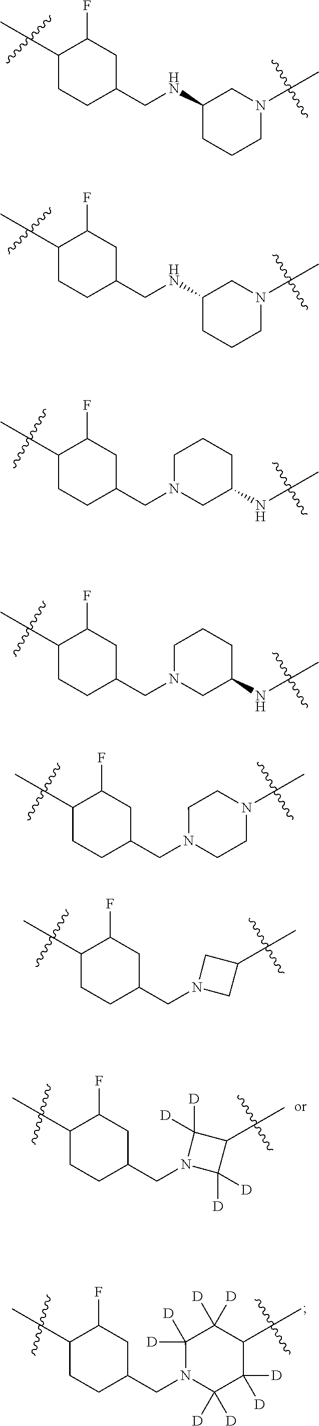
wherein the groups are optionally substituted with one or more substituents selected from the group consisting of deuterium, halogen, cyano, amino, C₁₋₆ alkyl, halogenated C₁₋₆ alkyl, deuterated C₁₋₆ alkyl, C₁₋₆ alkoxy, halogenated C₁₋₆ alkoxy, deuterated C₁₋₆ alkoxy, or any combination thereof.

36. The compound of Formula (III) or salts, enantiomers, stereoisomers, solvates, isotopically enriched analogs, prodrugs, or polymorphs thereof as claimed in claim 22, which is also of Formula (III-1), Formula (III-2), Formula (III-3), Formula (III-4), Formula (III-5), Formula (III-6), Formula (III-7), Formula (III-8), Formula (III-9), Formula (III-10), Formula (III-11), Formula (III-12), Formula (III-13), Formula (III-14), Formula (III-15), Formula (III-16), Formula (III-17), Formula (III-18), Formula (III-19), Formula (III-20), Formula (III-21), Formula (III-22), Formula (III-23) or Formula (III-24): 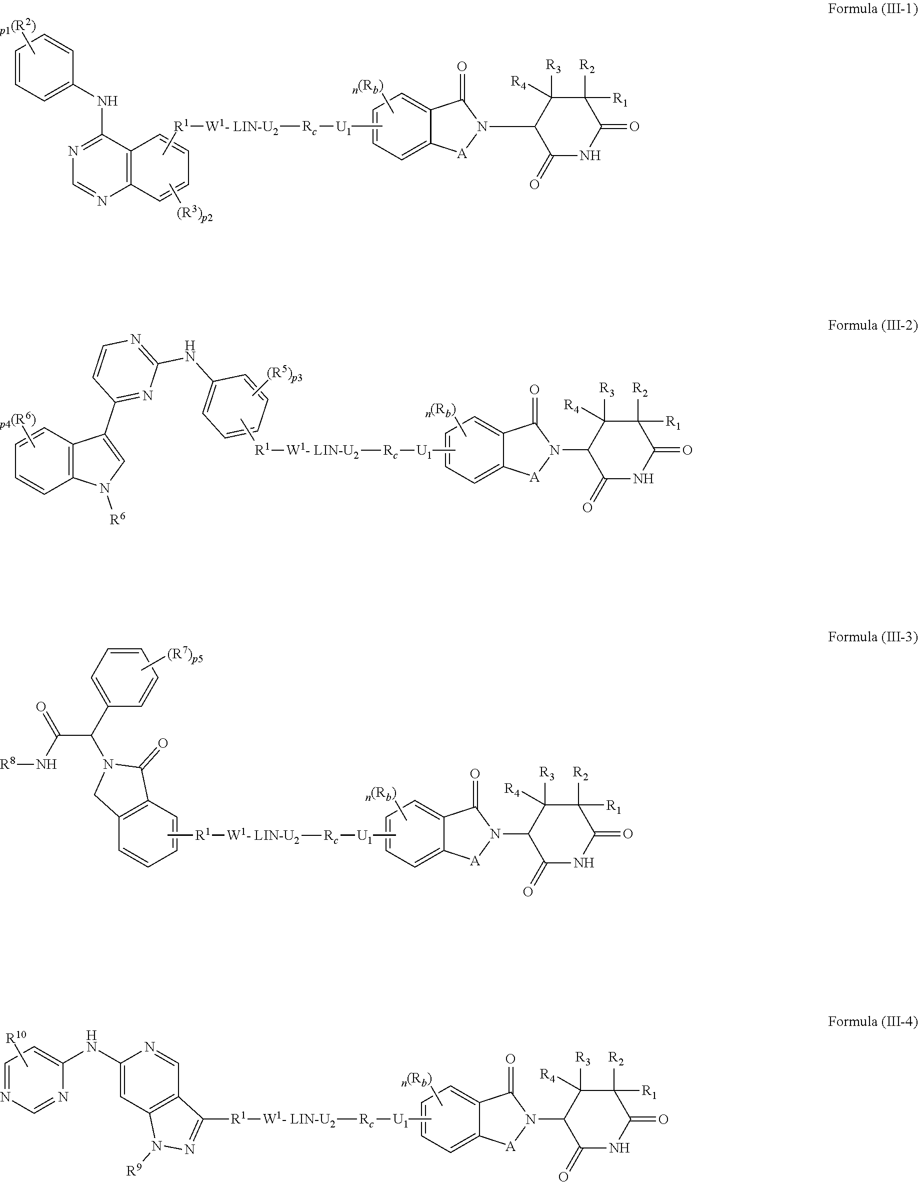 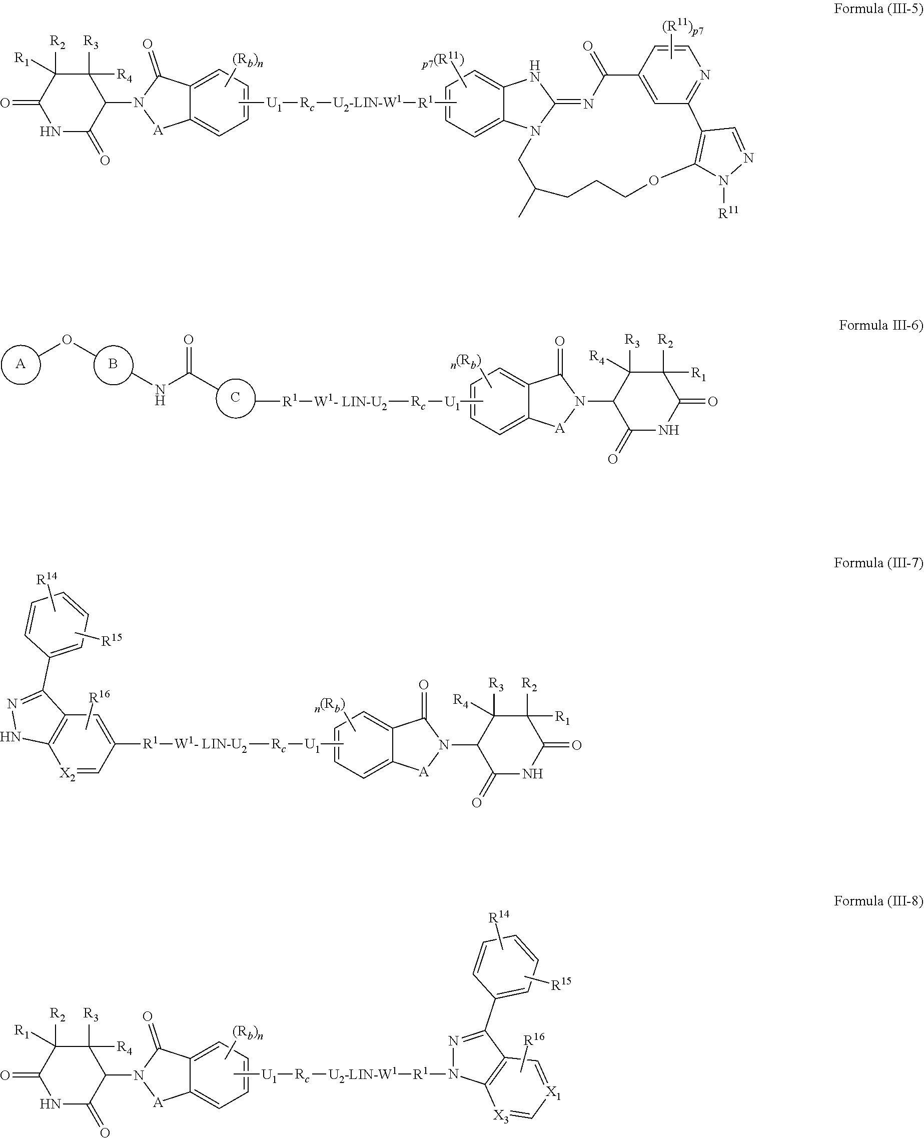 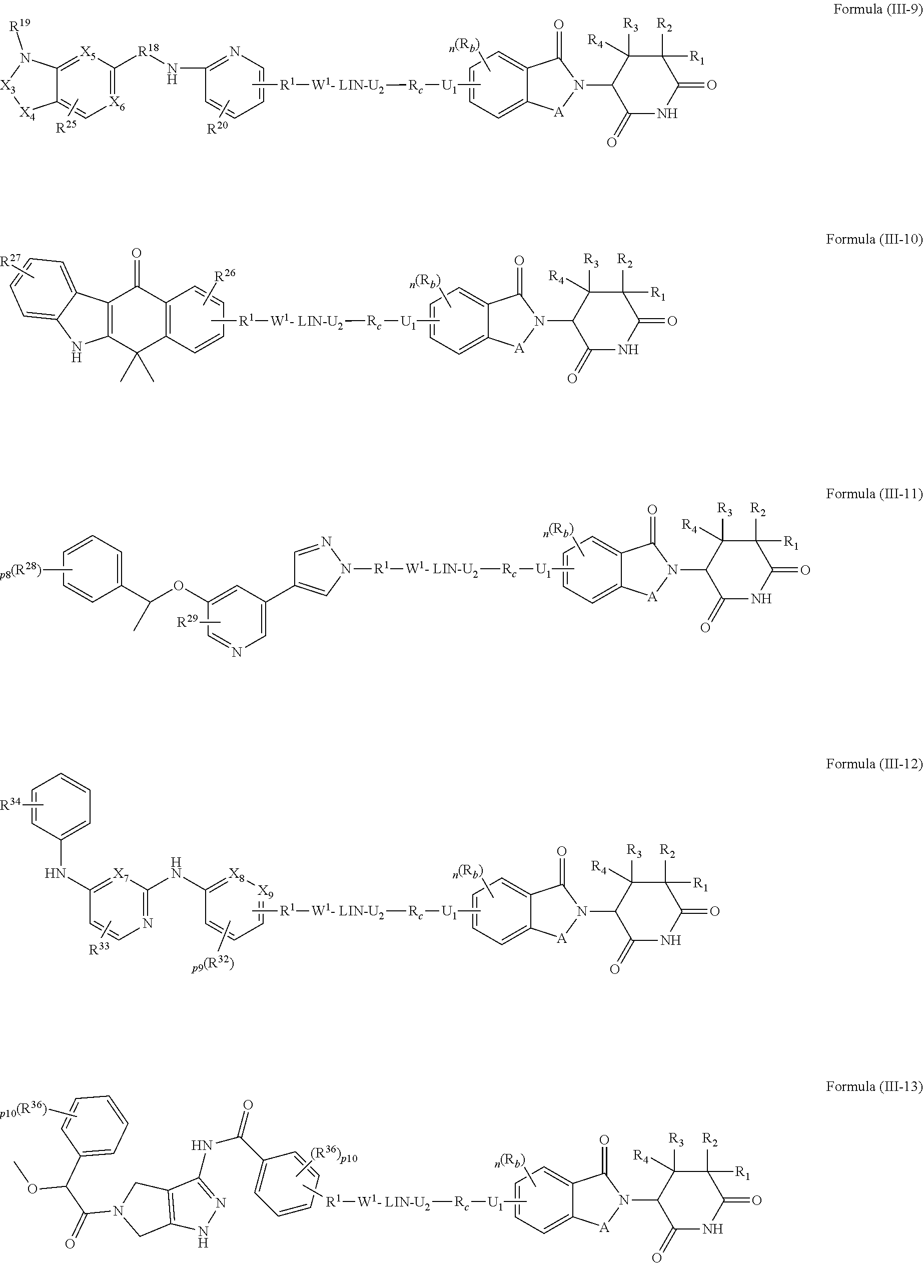 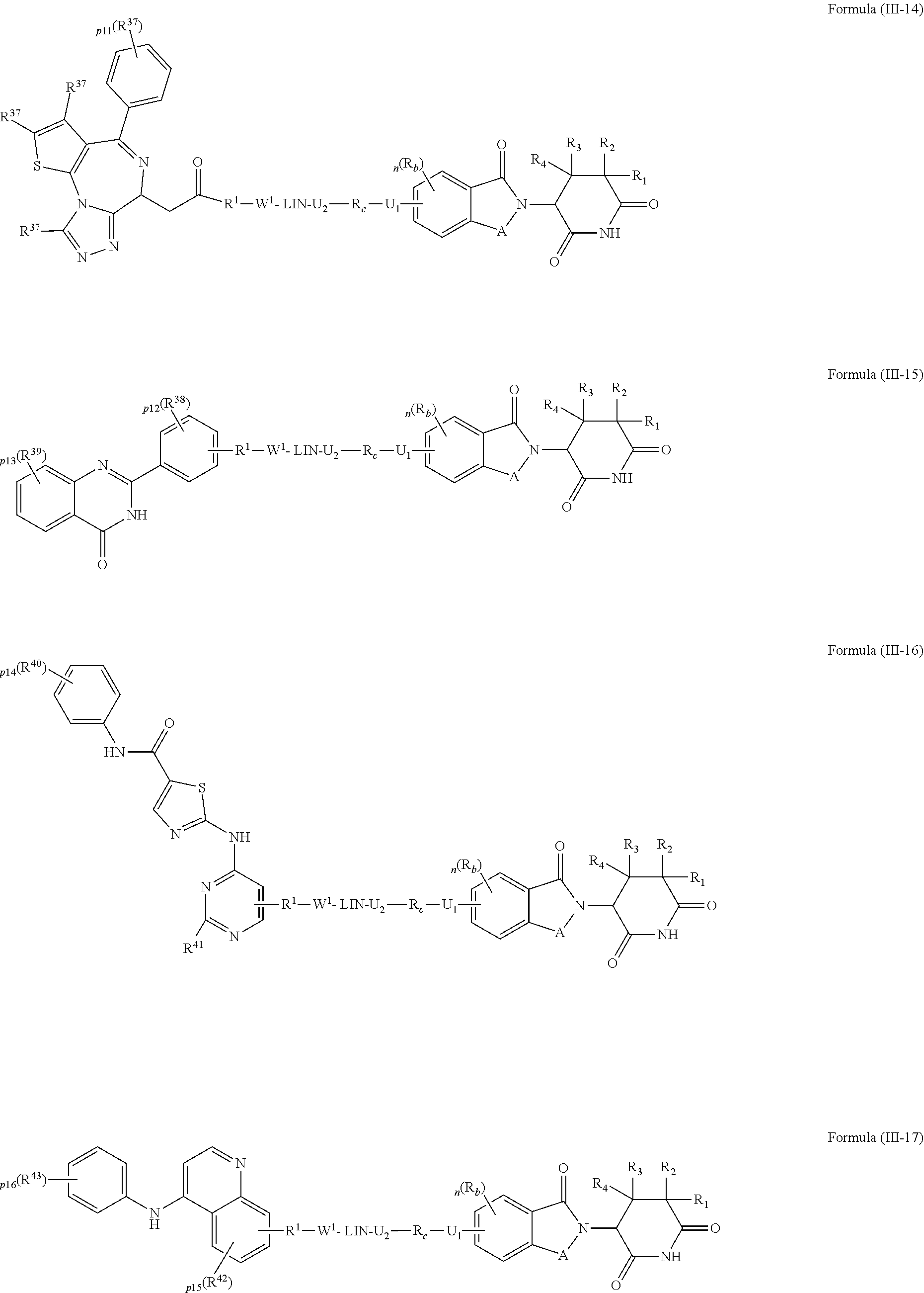 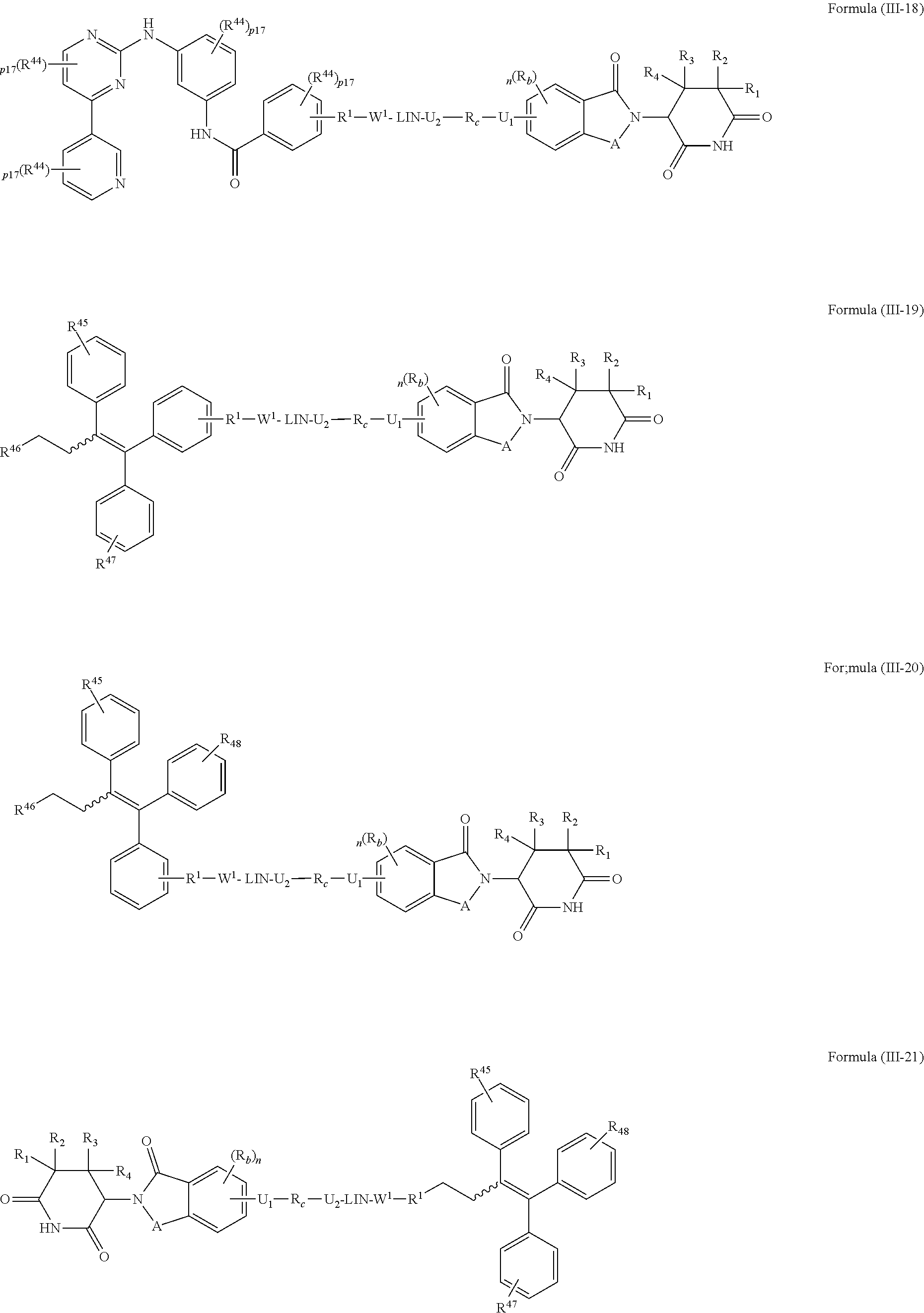 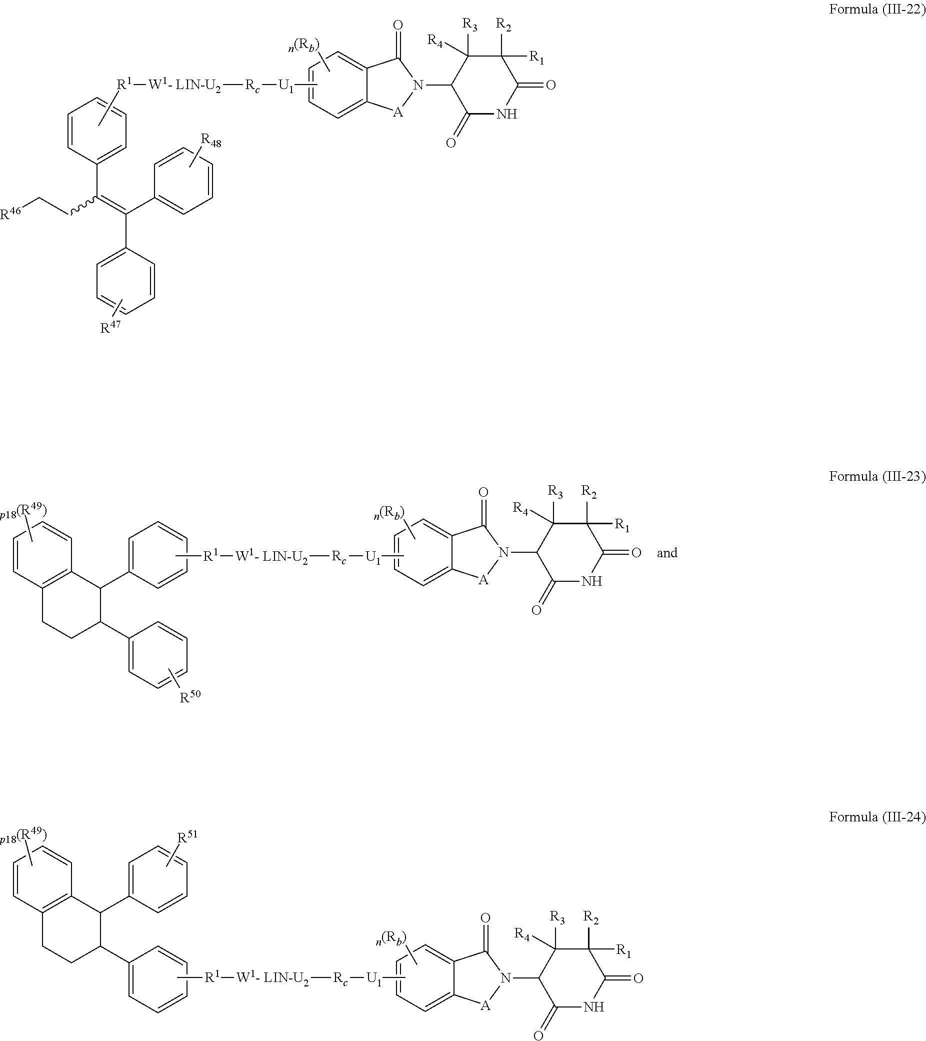
wherein A, R₁, R₂, R₃, R₄, (R_{b})ₙ, U₁, U₂, R_{c}, LIN, W¹, R¹, (R²)ₚ₁, (R³)ₚ₂, (R⁵)ₚ₃, (R⁶)ₚ₄, (R⁷)ₚ₅, R⁸, R⁹, R¹⁰, (R¹¹)ₚ₇, ring A, ring B, ring C, X₁, X₂, R¹⁴, R¹⁵, R¹⁶, X₃, X₄, X₅, X₆, R¹⁸, R¹⁹, R²⁰, R²⁵, R²⁶, R²⁷, (R²⁸)ₚ₈, R²⁹, X₇, X₈, X₉, (R³²)ₚ₉, R³³, R³⁴, (R³⁶)P₁₀, (R³⁷)ₚ₁₁, (R³⁸)ₚ₁₂, (R³⁹)ₚ₁₃, (R⁴⁰)ₚ₁₄, R⁴¹, (R⁴²)ₚ₁₅, (R⁴³)ₚ₁₆, (R⁴⁴)ₚ₁₇, R⁴⁵, R⁴⁶, R⁴⁷, R⁴⁸, (R⁴⁹)ₚ₁₈, R⁵⁰, and R⁵¹ are as defined in claim 22.

37. The compound of Formula (III) or salts, enantiomers, stereoisomers, solvates, isotopically enriched analogs, prodrugs, or polymorphs thereof as claimed in claim 20, which is selected from:
N-((1r,4r)-4-(3-chloro-4-cyanophenoxy)cyclohexyl)-6-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)pyridazine-3-carboxamide;
N-((1r,4r)-4-(3-chloro-4-cyanophenoxy)cyclohexyl)-6-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)pyridazine-3-carboxamide;
N-((1r,4r)-4-(3-chloro-4-cyanophenoxy)cyclohexyl)-6-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)pyridazine-3-carboxamide;
N-((1r,4r)-4-(3-chloro-4-cyanophenoxy)cyclohexyl)-6-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)pyridazine-3-carboxamide;
N-((1r,4r)-4-(3-chloro-4-cyanophenoxy)cyclohexyl)-6-(4-(((1S,4S)-5-(2-(2,6-dioxopiperidin-3-yl)-1,3-dioxoisoindolin-5-yl)-2,5-diazabicyclo[2.2.1]heptan-2-yl)methyl)piperidin-1-yl)pyridazine-3-carboxamide;
N-((1r,4r)-4-(3-chloro-4-cyanophenoxy)cyclohexyl)-6-(4-(((1R,4R)-5-(2-(2,6-dioxopiperidin-3-yl)-1,3-dioxoisoindolin-5-yl)-2,5-diazabicyclo[2.2.1]heptan-2-yl)methyl)piperidin-1-yl)pyridazine-3-carboxamide;
N-((1r,4r)-4-(3-chloro-4-cyanophenoxy)cyclohexyl)-6-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-1,3-dioxoisoindolin-5-yl)-3,5-dimethylpiperazin-1-yl)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-1,3-dioxoisoindolin-5-yl)-2,6-dimethylpiperazin-1-yl)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-1,3-dioxoisoindolin-5-yl)piperidin-1-yl)methyl)piperidin-1-yl)pyridazine-3-carboxamide;
N-((1r,4r)-4-(3-chloro-4-cyanophenoxy)cyclohexyl)-6-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)pyridazine-3-carboxamide;
N-((1r,4r)-4-(3-chloro-4-cyanophenoxy)cyclohexyl)-6-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)pyridazine-3-carboxamide;
N-((1r,4r)-4-(3-chloro-4-cyanophenoxy)cyclohexyl)-6-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)pyridazine-3-carboxamide;
N-((1r,4r)-4-(3-chloro-4-cyanophenoxy)cyclohexyl)-6-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)pyridazine-3-carboxamide;
N-((1r,4r)-4-(3-chloro-4-cyanophenoxy)cyclohexyl)-6-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-6-fluoro-1,3-dioxoisoindolin-5-yl)piperidin-1-yl)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)pyridazine-3-carboxamide;
N-((1r,4r)-4-(3-chloro-4-cyanophenoxy)cyclohexyl)-6-(4-((((3R)-1-(2-(2,6-dioxopiperidin-3-yl)-1,3-dioxoisoindolin-5-yl)piperidin-3-yl)amino)methyl)piperidin-1-yl)pyridazine-3-carboxamide;
N-((1r,4r)-4-(3-chloro-4-cyanophenoxy)cyclohexyl)-6-(4-((((3S)-1-(2-(2,6-dioxopiperidin-3-yl)-1,3-dioxoisoindolin-5-yl)piperidin-3-yl)amino)methyl)piperidin-1-yl)pyridazine-3-carboxamide;
N-((1r,4r)-4-(3-chloro-4-cyanophenoxy)cyclohexyl)-6-(4-(((3S)-3-((2-(2,6-dioxopiperidin-3-yl)-1,3-dioxoisoindolin-5-yl)amino)piperidin-1-yl)methyl)piperidin-1-yl)pyridazine-3-carboxamide;
N-((1r,4r)-4-(3-chloro-4-cyanophenoxy)cyclohexyl)-6-(4-(((3R)-3-((2-(2,6-dioxopiperidin-3-yl)-1,3-dioxoisoindolin-5-yl)amino)piperidin-1-yl)methyl)piperidin-1-yl)pyridazine-3-carboxamide;
N-((1r,4r)-4-(3-chloro-4-cyanophenoxy)cyclohexyl)-6-(3-((1-(2-(2,6-dioxopiperidin-3-yl)-6-fluoro-1,3-dioxoisoindolin-5-yl)piperidin-4-yl)methyl)-3,8-diazabicyclo[3.2.1]octan-8-yl)pyridazine-3-carboxamide;
N-((1r,4r)-4-(3-chloro-4-cyanophenoxy)cyclohexyl)-6-(8-((1-(2-(2,6-dioxopiperidin-3-yl)-6-fluoro-1,3-dioxoisoindolin-5-yl)piperidin-4-yl)methyl)-3,8-diazabicyclo[3.2.1]octan-3-yl)pyridazine-3-carboxamide;
N-((1r,4r)-4-(3-chloro-4-cyanophenoxy)cyclohexyl)-6-(3-((1-(2-(2,6-dioxopiperidin-3-yl)-6-fluoro-1,3-dioxoisoindolin-5-yl)piperidin-4-yl)methyl)-3,6-diazabicyclo[3.1.1]heptan-6-yl)pyridazine-3-carboxamide;
N-((1r,4r)-4-(3-chloro-4-cyanophenoxy)cyclohexyl)-6-(6-((1-(2-(2,6-dioxopiperidin-3-yl)-6-fluoro-1,3-dioxoisoindolin-5-yl)piperidin-4-yl)methyl)-3,6-diazabicyclo[3.1.1]heptan-3-yl)pyridazine-3-carboxamide;
N-((1r,3r)-3-(3-chloro-4-cyanophenoxy)-2,2,4,4-tetramethylcyclobutyl)-6-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)nicotinamide;
N-((1r,3r)-3-(3-chloro-4-cyanophenoxy)-2,2,4,4-tetramethylcyclobutyl)-6-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)nicotinamide;
N-((1r,3r)-3-(3-chloro-4-cyanophenoxy)-2,2,4,4-tetramethylcyclobutyl)-6-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)nicotinamide;
N-((1r,3r)-3-(3-chloro-4-cyanophenoxy)-2,2,4,4-tetramethylcyclobutyl)-6-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)nicotinamide;
N-((1r,3S)-3-(3-chloro-4-cyanophenoxy)-2,2,4,4-tetramethylcyclobutyl)-6-(4-(((1S,4S)-5-(2-(2,6-dioxopiperidin-3-yl)-1,3-dioxoisoindolin-5-yl)-2,5-diazabicyclo[2.2.1]heptan-2-yl)methyl)piperidin-1-yl)nicotinamide;
N-((1r,3R)-3-(3-chloro-4-cyanophenoxy)-2,2,4,4-tetramethylcyclobutyl)-6-(4-(((1R,4R)-5-(2-(2,6-dioxopiperidin-3-yl)-1,3-dioxoisoindolin-5-yl)-2,5-diazabicyclo[2.2.1]heptan-2-yl)methyl)piperidin-1-yl)nicotinamide;
N-((1r,3r)-3-(3-chloro-4-cyanophenoxy)-2,2,4,4-tetramethylcyclobutyl)-6-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-1,3-dioxoisoindolin-5-yl)-3,5-dimethylpiperazin-1-yl)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-1,3-dioxoisoindolin-5-yl)-2,6-dimethylpiperazin-1-yl)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-1,3-dioxoisoindolin-5-yl)piperidin-1-yl)methyl)piperidin-1-yl)nicotinamide;
N-((1r,3r)-3-(3-chloro-4-cyanophenoxy)-2,2,4,4-tetramethylcyclobutyl)-6-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)nicotinamide;
N-((1r,3r)-3-(3-chloro-4-cyanophenoxy)-2,2,4,4-tetramethylcyclobutyl)-6-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)nicotinamide;
N-((1r,3r)-3-(3-chloro-4-cyanophenoxy)-2,2,4,4-tetramethylcyclobutyl)-6-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)nicotinamide;
N-((1r,3r)-3-(3-chloro-4-cyanophenoxy)-2,2,4,4-tetramethylcyclobutyl)-6-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)nicotinamide;
N-((1r,3r)-3-(3-chloro-4-cyanophenoxy)-2,2,4,4-tetramethylcyclobutyl)-6-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-6-fluoro-1,3-dioxoisoindolin-5-yl)piperidin-1-yl)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)nicotinamide;
N-((1r,3R)-3-(3-chloro-4-cyanophenoxy)-2,2,4,4-tetramethylcyclobutyl)-6-(4-((((3R)-1-(2-(2,6-dioxopiperidin-3-yl)-1,3-dioxoisoindolin-5-yl)piperidin-3-yl)amino)methyl)piperidin-1-yl)nicotinamide;
N-((1r,3S)-3-(3-chloro-4-cyanophenoxy)-2,2,4,4-tetramethylcyclobutyl)-6-(4-((((3S)-1-(2-(2,6-dioxopiperidin-3-yl)-1,3-dioxoisoindolin-5-yl)piperidin-3-yl)amino)methyl)piperidin-1-yl)nicotinamide;
N-((1r,3r)-3-(3-chloro-4-cyanophenoxy)-2,2,4,4-tetramethylcyclobutyl)-6-(4-(((3S)-3-((2-(2,6-dioxopiperidin-3-yl)-1,3-dioxoisoindolin-5-yl)amino)piperidin-1-yl)methyl)piperidin-1-yl)nicotinamide;
N-((1r,3r)-3-(3-chloro-4-cyanophenoxy)-2,2,4,4-tetramethylcyclobutyl)-6-(4-(((3R)-3-((2-(2,6-dioxopiperidin-3-yl)-1,3-dioxoisoindolin-5-yl)amino)piperidin-1-yl)methyl)piperidin-1-yl)nicotinamide;
N-((1r,3r)-3-(3-chloro-4-cyanophenoxy)-2,2,4,4-tetramethylcyclobutyl)-6-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)pyridazine-3-carboxamide;
N-((1r,3r)-3-(3-chloro-4-cyanophenoxy)-2,2,4,4-tetramethylcyclobutyl)-6-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)pyridazine-3-carboxamide;
N-((1r,3r)-3-(3-chloro-4-cyanophenoxy)-2,2,4,4-tetramethylcyclobutyl)-6-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)pyridazine-3-carboxamide;
N-((1r,3r)-3-(3-chloro-4-cyanophenoxy)-2,2,4,4-tetramethylcyclobutyl)-6-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)pyridazine-3-carboxamide;
5-(4-((4-(4-amino-3-(4-phenoxyphenyl)-1H-pyrazolo[3,4-d]pyrimidin-1-yl)cyclohexyl)methyl)piperazin-1-yl)-2-(2,6-dioxopiperidin-3-yl)isoindoline-1,3-dione;
5-(4-((4-(4-amino-3-(4-phenoxyphenyl)-1H-pyrazolo[3,4-d]pyrimidin-1-yl)-3-fluorocyclohexyl)methyl)piperazin-1-yl)-2-(2,6-dioxopiperidin-3-yl)isoindoline-1,3-dione;
5-(3-((4-(4-amino-3-(4-phenoxyphenyl)-1H-pyrazolo[3,4-d]pyrimidin-1-yl)cyclohexyl)methyl)-3,6-diazabicyclo[3.1.1]heptan-6-yl)-2-(2,6-dioxopiperidin-3-yl)isoindoline-1,3-dione;
5-(3-((4-(4-amino-3-(4-phenoxyphenyl)-1H-pyrazolo[3,4-d]pyrimidin-1-yl)-3-fluorocyclohexyl)methyl)-3,6-diazabicyclo[3.1.1]heptan-6-yl)-2-(2,6-dioxopiperidin-3-yl)isoindoline-1,3-dione;
5-(6-((4-(4-amino-3-(4-phenoxyphenyl)-1H-pyrazolo[3,4-d]pyrimidin-1-yl)cyclohexyl)methyl)-3,6-diazabicyclo[3.1.1]heptan-3-yl)-2-(2,6-dioxopiperidin-3-yl)isoindoline-1,3-dione;
5-(6-((4-(4-amino-3-(4-phenoxyphenyl)-1H-pyrazolo[3,4-d]pyrimidin-1-yl)-3-fluorocyclohexyl)methyl)-3,6-diazabicyclo[3.1.1]heptan-3-yl)-2-(2,6-dioxopiperidin-3-5-(3-((4-(4-amino-3-(4-phenoxyphenyl)-1H-pyrazolo[3,4-d]pyrimidin-1-yl)cyclohexyl)methyl)-3,8-diazabicyclo[3.2.1]octan-8-yl)-2-(2,6-dioxopiperidin-3-yl)isoindoline-1,3-dione;
5-(3-((4-(4-amino-3-(4-phenoxyphenyl)-1H-pyrazolo[3,4-d]pyrimidin-1-yl)-3-fluorocyclohexyl)methyl)-3,8-diazabicyclo[3.2.1]octan-8-yl)-2-(2,6-dioxopiperidin-3-yl)isoindoline-1,3-dione;
5-(8-((4-(4-amino-3-(4-phenoxyphenyl)-1H-pyrazolo[3,4-d]pyrimidin-1-yl)cyclohexyl)methyl)-3,8-diazabicyclo[3.2.1]octan-3-yl)-2-(2,6-dioxopiperidin-3-yl)isoindoline-1,3-dione;
5-(8-((4-(4-amino-3-(4-phenoxyphenyl)-1H-pyrazolo[3,4-d]pyrimidin-1-yl)-3-fluorocyclohexyl)methyl)-3,8-diazabicyclo[3.2.1]octan-3-yl)-2-(2,6-dioxopiperidin-3-yl)isoindoline-1,3-dione;
5-((1S,4S)-5-((4-(4-amino-3-(4-phenoxyphenyl)-1H-pyrazolo[3,4-d]pyrimidin-1-yl)cyclohexyl)methyl)-2,5-diazabicyclo[2.2.1]heptan-2-yl)-2-(2,6-dioxopiperidin-3-yl)isoindoline-1,3-dione;
5-((1S,4S)-5-((4-(4-amino-3-(4-phenoxyphenyl)-1H-pyrazolo[3,4-d]pyrimidin-1-yl)-3-fluorocyclohexyl)methyl)-2,5-diazabicyclo[2.2.1]heptan-2-yl)-2-(2,6-dioxopiperidin-3-yl)isoindoline-1,3-dione;
5-((1R,4R)-5-((4-(4-amino-3-(4-phenoxyphenyl)-1H-pyrazolo[3,4-d]pyrimidin-1-yl)cyclohexyl)methyl)-2,5-diazabicyclo[2.2.1]heptan-2-yl)-2-(2,6-dioxopiperidin-3-yl)isoindoline-1,3-dione;
5-((1R,4R)-5-((4-(4-amino-3-(4-phenoxyphenyl)-1H-pyrazolo[3,4-d]pyrimidin-1-yl)-3-fluorocyclohexyl)methyl)-2,5-diazabicyclo[2.2.1]heptan-2-yl)-2-(2,6-dioxopiperidin-3-yl)isoindoline-1,3-dione;
5-(5-((4-(4-amino-3-(4-phenoxyphenyl)-1H-pyrazolo[3,4-d]pyrimidin-1-yl)cyclohexyl)methyl)-2,5-diazabicyclo[2.2.2]octan-2-yl)-2-(2,6-dioxopiperidin-3-yl)isoindoline-1,3-dione;
5-(5-((4-(4-amino-3-(4-phenoxyphenyl)-1H-pyrazolo[3,4-d]pyrimidin-1-yl)-3-fluorocyclohexyl)methyl)-2,5-diazabicyclo[2.2.2]octan-2-yl)-2-(2,6-dioxopiperidin-3-yl)isoindoline-1,3-dione;
5-(4-((4-(4-amino-3-(4-phenoxyphenyl)-1H-pyrazolo[3,4-d]pyrimidin-1-yl)cyclohexyl)methyl)piperazin-1-yl-2,2,3,3,5,5,6,6-d₈)-2-(2,6-dioxopiperidin-3-yl)isoindoline-1,3-dione;
5-(4-((4-(4-amino-3-(4-phenoxyphenyl)-1H-pyrazolo[3,4-d]pyrimidin-1-yl)-3-fluorocyclohexyl)methyl)piperazin-1-yl-2,2,3,3,5,5,6,6-d₈)-2-(2,6-dioxopiperidin-3-yl)isoindoline-1,3-dione;
5-(4-((4-(4-amino-3-(4-phenoxyphenyl)-1H-pyrazolo[3,4-d]pyrimidin-1-yl)cyclohexyl)methyl)-2,6-dimethylpiperazin-1-yl)-2-(2,6-dioxopiperidin-3-yl)isoindoline-1,3-dione;
5-(4-((4-(4-amino-3-(4-phenoxyphenyl)-1H-pyrazolo[3,4-d]pyrimidin-1-yl)-3-fluorocyclohexyl)methyl)-2,6-dimethylpiperazin-1-yl)-2-(2,6-dioxopiperidin-3-yl)isoindoline-1,3-dione;
5-(4-((4-(4-amino-3-(4-phenoxyphenyl)-1H-pyrazolo[3,4-d]pyrimidin-1-yl)cyclohexyl)methyl)-3,5-dimethylpiperazin-1-yl)-2-(2,6-dioxopiperidin-3-yl)isoindoline-1,3-dione;
5-(4-((4-(4-amino-3-(4-phenoxyphenyl)-1H-pyrazolo[3,4-d]pyrimidin-1-yl)-3-fluorocyclohexyl)methyl)-3,5-dimethylpiperazin-1-yl)-2-(2,6-dioxopiperidin-3-yl)isoindoline-1,3-dione;
5-(1-((4-(4-amino-3-(4-phenoxyphenyl)-1H-pyrazolo[3,4-d]pyrimidin-1-yl)cyclohexyl)methyl)piperidin-4-yl)-2-(2,6-dioxopiperidin-3-yl)isoindoline-1,3-dione;
5-(1-((4-(4-amino-3-(4-phenoxyphenyl)-1H-pyrazolo[3,4-d]pyrimidin-1-yl)-3-fluorocyclohexyl)methyl)piperidin-4-yl)-2-(2,6-dioxopiperidin-3-yl)isoindoline-1,3-dione;
5-(4-((4-(4-amino-3-(4-phenoxyphenyl)-1H-pyrazolo[3,4-d]pyrimidin-1-yl)cyclohexyl)methyl)piperazin-1-yl)-2-(2,6-dioxopiperidin-3-yl)-6-fluoroisoindoline-1,3-dione;
5-(4-((4-(4-amino-3-(4-phenoxyphenyl)-1H-pyrazolo[3,4-d]pyrimidin-1-yl)-3-fluorocyclohexyl)methyl)piperazin-1-yl)-2-(2,6-dioxopiperidin-3-yl)-6-fluoroisoindoline-1,3-dione;
5-(3-((4-(4-amino-3-(4-phenoxyphenyl)-1H-pyrazolo[3,4-d]pyrimidin-1-yl)cyclohexyl)methyl)-3,6-diazabicyclo[3.1.1]heptan-6-yl)-2-(2,6-dioxopiperidin-3-yl)-6-fluoroisoindoline-1,3-dione;
5-(3-((4-(4-amino-3-(4-phenoxyphenyl)-1H-pyrazolo[3,4-d]pyrimidin-1-yl)-3-fluorocyclohexyl)methyl)-3,6-diazabicyclo[3.1.1]heptan-6-yl)-2-(2,6-dioxopiperidin-3-yl)-6-fluoroisoindoline-1,3-dione;
5-(6-((4-(4-amino-3-(4-phenoxyphenyl)-1H-pyrazolo[3,4-d]pyrimidin-1-yl)cyclohexyl)methyl)-3,6-diazabicyclo[3.1.1]heptan-3-yl)-2-(2,6-dioxopiperidin-3-yl)-6-fluoroisoindoline-1,3-dione;
5-(6-((4-(4-amino-3-(4-phenoxyphenyl)-1H-pyrazolo[3,4-d]pyrimidin-1-yl)-3-fluorocyclohexyl)methyl)-3,6-diazabicyclo[3.1.1]heptan-3-yl)-2-(2,6-dioxopiperidin-3-yl)-6-fluoroisoindoline-1,3-dione;
5-(3-((4-(4-amino-3-(4-phenoxyphenyl)-1H-pyrazolo[3,4-d]pyrimidin-1-yl)cyclohexyl)methyl)-3,8-diazabicyclo[3.2.1]octan-8-yl)-2-(2,6-dioxopiperidin-3-yl)-6-fluoroisoindoline-1,3-dione;
5-(3-((4-(4-amino-3-(4-phenoxyphenyl)-1H-pyrazolo[3,4-d]pyrimidin-1-yl)-3-fluorocyclohexyl)methyl)-3,8-diazabicyclo[3.2.1]octan-8-yl)-2-(2,6-dioxopiperidin-3-yl)-6-fluoroisoindoline-1,3-dione;
5-(8-((4-(4-amino-3-(4-phenoxyphenyl)-1H-pyrazolo[3,4-d]pyrimidin-1-yl)cyclohexyl)methyl)-3,8-diazabicyclo[3.2.1]octan-3-yl)-2-(2,6-dioxopiperidin-3-yl)-6-fluoroisoindoline-1,3-dione;
5-(8-((4-(4-amino-3-(4-phenoxyphenyl)-1H-pyrazolo[3,4-d]pyrimidin-1-yl)-3-fluorocyclohexyl)methyl)-3,8-diazabicyclo[3.2.1]octan-3-yl)-2-(2,6-dioxopiperidin-3-yl)-6-fluoroisoindoline-1,3-dione;
5-((1S,4S)-5-((4-(4-amino-3-(4-phenoxyphenyl)-1H-pyrazolo[3,4-d]pyrimidin-1-yl)cyclohexyl)methyl)-2,5-diazabicyclo[2.2.1]heptan-2-yl)-2-(2,6-dioxopiperidin-3-yl)-6-fluoroisoindoline-1,3-dione;
5-((1S,4S)-5-((4-(4-amino-3-(4-phenoxyphenyl)-1H-pyrazolo[3,4-d]pyrimidin-1-yl)-3-fluorocyclohexyl)methyl)-2,5-diazabicyclo[2.2.1]heptan-2-yl)-2-(2,6-dioxopiperidin-3-yl)-6-fluoroisoindoline-1,3-dione;
5-((1R,4R)-5-((4-(4-amino-3-(4-phenoxyphenyl)-1H-pyrazolo[3,4-d]pyrimidin-1-yl)cyclohexyl)methyl)-2,5-diazabicyclo[2.2.1]heptan-2-yl)-2-(2,6-dioxopiperidin-3-yl)-6-fluoroisoindoline-1,3-dione;
5-((1R,4R)-5-((4-(4-amino-3-(4-phenoxyphenyl)-1H-pyrazolo[3,4-d]pyrimidin-1-yl)-3-fluorocyclohexyl)methyl)-2,5-diazabicyclo[2.2.1]heptan-2-yl)-2-(2,6-dioxopiperidin-3-yl)-6-fluoroisoindoline-1,3-dione;
5-(5-((4-(4-amino-3-(4-phenoxyphenyl)-1H-pyrazolo[3,4-d]pyrimidin-1-yl)cyclohexyl)methyl)-2,5-diazabicyclo[2.2.2]octan-2-yl)-2-(2,6-dioxopiperidin-3-yl)-6-fluoroisoindoline-1,3-dione;
5-(5-((4-(4-amino-3-(4-phenoxyphenyl)-1H-pyrazolo[3,4-d]pyrimidin-1-yl)-3-fluorocyclohexyl)methyl)-2,5-diazabicyclo[2.2.2]octan-2-yl)-2-(2,6-dioxopiperidin-3-yl)-6-fluoroisoindoline-1,3-dione;
5-(4-((4-(4-amino-3-(4-phenoxyphenyl)-1H-pyrazolo[3,4-d]pyrimidin-1-yl)cyclohexyl)methyl)-2,6-dimethylpiperazin-1-yl)-2-(2,6-dioxopiperidin-3-yl)-6-fluoroisoindoline-1,3-dione;
5-(4-((4-(4-amino-3-(4-phenoxyphenyl)-1H-pyrazolo[3,4-d]pyrimidin-1-yl)-3-fluorocyclohexyl)methyl)-2,6-dimethylpiperazin-1-yl)-2-(2,6-dioxopiperidin-3-yl)-6-fluoroisoindoline-1,3-dione;
5-(4-((4-(4-amino-3-(4-phenoxyphenyl)-1H-pyrazolo[3,4-d]pyrimidin-1-yl)cyclohexyl)methyl)-3,5-dimethylpiperazin-1-yl)-2-(2,6-dioxopiperidin-3-yl)-6-fluoroisoindoline-1,3-dione;
5-(4-((4-(4-amino-3-(4-phenoxyphenyl)-1H-pyrazolo[3,4-d]pyrimidin-1-yl)-3-fluorocyclohexyl)methyl)-3,5-dimethylpiperazin-1-yl)-2-(2,6-dioxopiperidin-3-yl)-6-fluoroisoindoline-1,3-dione;
5-(1-((4-(4-amino-3-(4-phenoxyphenyl)-1H-pyrazolo[3,4-d]pyrimidin-1-yl)cyclohexyl)methyl)piperidin-4-yl)-2-(2,6-dioxopiperidin-3-yl)-6-fluoroisoindoline-1,3-dione;
5-(1-((4-(4-amino-3-(4-phenoxyphenyl)-1H-pyrazolo[3,4-d]pyrimidin-1-yl)-3-fluorocyclohexyl)methyl)piperidin-4-yl)-2-(2,6-dioxopiperidin-3-yl)-6-fluoroisoindoline-1,3-dione;
5-(4-((4-(4-amino-3-(4-phenoxyphenyl)-1H-pyrazolo[3,4-d]pyrimidin-1-yl)cyclohexyl)methyl)piperazin-1-yl-2,2,3,3,5,5,6,6-d₈)-2-(2,6-dioxopiperidin-3-yl)-6-fluoroisoindoline-1,3-dione;
5-(4-((4-(4-amino-3-(4-phenoxyphenyl)-1H-pyrazolo[3,4-d]pyrimidin-1-yl)-3-fluorocyclohexyl)methyl)piperazin-1-yl-2,2,3,3,5,5,6,6-d₈)-2-(2,6-dioxopiperidin-3-yl)-6-fluoroisoindoline-1,3-dione;
6-(4-((4-(4-amino-3-(4-phenoxyphenyl)-1H-pyrazolo[3,4-d]pyrimidin-1-yl)cyclohexyl)methyl)piperazin-1-yl-2,2,3,3,5,5,6,6-d₈)-2-(2,6-dioxopiperidin-3-yl)-4-fluoroisoindoline-1,3-dione;
6-(4-((4-(4-amino-3-(4-phenoxyphenyl)-1H-pyrazolo[3,4-d]pyrimidin-1-yl)-3-fluorocyclohexyl)methyl)piperazin-1-yl-2,2,3,3,5,5,6,6-d₈)-2-(2,6-dioxopiperidin-3-yl)-4-fluoroisoindoline-1,3-dione;
5-(4-((4-(4-amino-3-(4-phenoxyphenyl)-1H-pyrazolo[3,4-d]pyrimidin-1-yl)cyclohexyl)methyl)piperazin-1-yl-2,2,3,3,5,5,6,6-d₈)-2-(2,6-dioxopiperidin-3-yl)-4-fluoroisoindoline-1,3-dione;
5-(4-((4-(4-amino-3-(4-phenoxyphenyl)-1H-pyrazolo[3,4-d]pyrimidin-1-yl)-3-fluorocyclohexyl)methyl)piperazin-1-yl-2,2,3,3,5,5,6,6-d₈)-2-(2,6-dioxopiperidin-3-yl)-4-fluoroisoindoline-1,3-dione;
5-(4-((4-(4-amino-3-(4-phenoxyphenyl)-1H-pyrazolo[3,4-d]pyrimidin-1-yl)cyclohexyl)methyl)piperazin-1-yl-2,2,3,3,5,5,6,6-d₈)-2-(2,6-dioxopiperidin-3-yl)-4,7-difluoroisoindoline-1,3-dione;
5-(4-((4-(4-amino-3-(4-phenoxyphenyl)-1H-pyrazolo[3,4-d]pyrimidin-1-yl)-3-fluorocyclohexyl)methyl)piperazin-1-yl-2,2,3,3,5,5,6,6-d₈)-2-(2,6-dioxopiperidin-3-yl)-4,7-difluoroisoindoline-1,3-dione;
5-(4-((4-(4-amino-3-(4-phenoxyphenyl)-1H-pyrazolo[3,4-d]pyrimidin-1-yl)cyclohexyl)methyl)piperazin-1-yl-2,2,3,3,5,5,6,6-d₈)-2-(2,6-dioxopiperidin-3-yl)-4,6-difluoroisoindoline-1,3-dione;
5-(4-((4-(4-amino-3-(4-phenoxyphenyl)-1H-pyrazolo[3,4-d]pyrimidin-1-yl)-3-fluorocyclohexyl)methyl)piperazin-1-yl-2,2,3,3,5,5,6,6-d₈)-2-(2,6-dioxopiperidin-3-yl)-4,6-difluoroisoindoline-1,3-dione;
6-(4-((4-(4-amino-3-(4-phenoxyphenyl)-1H-pyrazolo[3,4-d]pyrimidin-1-yl)cyclohexyl)methyl)piperazin-1-yl-2,2,3,3,5,5,6,6-d₈)-2-(2,6-dioxopiperidin-3-yl)-4,5-difluoroisoindoline-1,3-dione;
6-(4-((4-(4-amino-3-(4-phenoxyphenyl)-1H-pyrazolo[3,4-d]pyrimidin-1-yl)-3-fluorocyclohexyl)methyl)piperazin-1-yl-2,2,3,3,5,5,6,6-d₈)-2-(2,6-dioxopiperidin-3-yl)-4,5-difluoroisoindoline-1,3-dione;
5-(4-((4-(4-amino-3-(4-phenoxyphenyl)-1H-pyrazolo[3,4-d]pyrimidin-1-yl)cyclohexyl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((4-(4-amino-3-(4-phenoxyphenyl)-1H-pyrazolo[3,4-d]pyrimidin-1-yl)-3-fluorocyclohexyl)methyl)piperazin-1-yl-2,2,3,3,5,5,6,6-d₈)-2-(2,6-dioxopiperidin-3-yl)-4,6,7-trifluoroisoindoline-1,3-dione;
5-((R)-3-(((4-(4-amino-3-(4-phenoxyphenyl)-1H-pyrazolo[3,4-d]pyrimidin-1-yl)cyclohexyl)methyl)amino)piperidin-1-yl)-2-(2,6-dioxopiperidin-3-yl)isoindoline-1,3-dione;
5-((3R)-3-(((4-(4-amino-3-(4-phenoxyphenyl)-1H-pyrazolo[3,4-d]pyrimidin-1-yl)-3-fluorocyclohexyl)methyl)amino)piperidin-1-yl)-2-(2,6-dioxopiperidin-3-yl)isoindoline-1,3-dione;
5-((S)-3-(((4-(4-amino-3-(4-phenoxyphenyl)-1H-pyrazolo[3,4-d]pyrimidin-1-yl)cyclohexyl)methyl)amino)piperidin-1-yl)-2-(2,6-dioxopiperidin-3-yl)isoindoline-1,3-dione;
5-((3S)-3-(((4-(4-amino-3-(4-phenoxyphenyl)-1H-pyrazolo[3,4-d]pyrimidin-1-yl)-3-fluorocyclohexyl)methyl)amino)piperidin-1-yl)-2-(2,6-dioxopiperidin-3-yl)isoindoline-1,3-dione;
5-(((S)-1-((4-(4-amino-3-(4-phenoxyphenyl)-1H-pyrazolo[3,4-d]pyrimidin-1-yl)cyclohexyl)methyl)piperidin-3-yl)amino)-2-(2,6-dioxopiperidin-3-yl)isoindoline-1,3-dione;
5-(((3S)-1-((4-(4-amino-3-(4-phenoxyphenyl)-1H-pyrazolo[3,4-d]pyrimidin-1-yl)-3-fluorocyclohexyl)methyl)piperidin-3-yl)amino)-2-(2,6-dioxopiperidin-3-yl)isoindoline-1,3-dione;
5-(((R)-1-((4-(4-amino-3-(4-phenoxyphenyl)-1H-pyrazolo[3,4-d]pyrimidin-1-yl)cyclohexyl)methyl)piperidin-3-yl)amino)-2-(2,6-dioxopiperidin-3-yl)isoindoline-1,3-dione;
5-(((3R)-1-((4-(4-amino-3-(4-phenoxyphenyl)-1H-pyrazolo[3,4-d]pyrimidin-1-yl)-3-fluorocyclohexyl)methyl)piperidin-3-yl)amino)-2-(2,6-dioxopiperidin-3-yl)isoindoline-1,3-dione;
3-(tert-butyl)-N-(4-(5-(4-(4-((4-(2-(2,6-dioxopiperidin-3-yl)-1,3-dioxoisoindolin-5-yl)piperazin-1-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-(((1S,4S)-5-(2-(2,6-dioxopiperidin-3-yl)-1,3-dioxoisoindolin-5-yl)-2,5-diazabicyclo[2.2.1]heptan-2-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-(((1R,4R)-5-(2-(2,6-dioxopiperidin-3-yl)-1,3-dioxoisoindolin-5-yl)-2,5-diazabicyclo[2.2.1]heptan-2-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-1,3-dioxoisoindolin-5-yl)-3,5-dimethylpiperazin-1-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-1,3-dioxoisoindolin-5-yl)-2,6-dimethylpiperazin-1-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-1,3-dioxoisoindolin-5-yl)piperidin-1-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-6-fluoro-1,3-dioxoisoindolin-5-yl)piperazin-1-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-(((1S,4S)-5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-(((1R,4R)-5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((4-(2-(2,6-dioxopiperidin-3-yl)-6-fluoro-1,3-dioxoisoindolin-5-yl)piperidin-1-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((((3R)-1-(2-(2,6-dioxopiperidin-3-yl)-1,3-dioxoisoindolin-5-yl)piperidin-3-yl)amino)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-((((3S)-1-(2-(2,6-dioxopiperidin-3-yl)-1,3-dioxoisoindolin-5-yl)piperidin-3-yl)amino)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-(((3S)-3-((2-(2,6-dioxopiperidin-3-yl)-1,3-dioxoisoindolin-5-yl)amino)piperidin-1-yl)methyl)piperidin-1-yl)phenyl)-1H-indazol-3-yl)-2-methylbenzyl)-1,2,4-oxadiazole-5-carboxamide;
3-(tert-butyl)-N-(4-(5-(4-(4-(((3R)-3-((2-(2,6-dioxopiperidin-3-yl)-1,3-dioxoisoindolin-5-yl)amino)piperidin-1-yl)methyl)piperidin-1-yl)phenyl)-1H-indazol-3-yl)-2-methylbenzyl)-1,2,4-oxadiazole-5-carboxamide;
2-(2,6-dioxopiperidin-3-yl)-5-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperazin-1-yl)isoindoline-1,3-dione;
2-(2,6-dioxopiperidin-3-yl)-5-fluoro-6-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperazin-1-yl)isoindoline-1,3-dione;
2-(2,6-dioxopiperidin-3-yl)-5-(3-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-3,6-diazabicyclo[3.1.1]heptan-6-yl)isoindoline-1,3-dione;
2-(2,6-dioxopiperidin-3-yl)-5-fluoro-6-(3-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-3,6-diazabicyclo[3.1.1]heptan-6-yl)isoindoline-1,3-dione;
2-(2,6-dioxopiperidin-3-yl)-5-(6-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-3,6-diazabicyclo[3.1.1]heptan-3-yl)isoindoline-1,3-dione;
2-(2,6-dioxopiperidin-3-yl)-5-fluoro-6-(6-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-3,6-diazabicyclo[3.1.1]heptan-3-yl)isoindoline-1,3-dione;
2-(2,6-dioxopiperidin-3-yl)-5-(3-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-3,8-diazabicyclo[3.2.1]octan-8-yl)isoindoline-1,3-dione;
2-(2,6-dioxopiperidin-3-yl)-5-fluoro-6-(3-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-3,8-diazabicyclo[3.2.1]octan-8-yl)isoindoline-1,3-dione;
2-(2,6-dioxopiperidin-3-yl)-5-fluoro-6-(8-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-3,8-diazabicyclo[3.2.1]octan-3-yl)isoindoline-1,3-dione;
2-(2,6-dioxopiperidin-3-yl)-5-(8-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-3,8-diazabicyclo[3.2.1]octan-3-yl)isoindoline-1,3-dione;
2-(2,6-dioxopiperidin-3-yl)-5-((1S,4S)-5-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-2,5-diazabicyclo[2.2.1]heptan-2-yl)isoindoline-1,3-dione;
2-(2,6-dioxopiperidin-3-yl)-5-fluoro-6-((1S,4S)-5-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-2,5-diazabicyclo[2.2.1]heptan-2-yl)isoindoline-1,3-dione;
2-(2,6-dioxopiperidin-3-yl)-5-((1R,4R)-5-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-2,5-diazabicyclo[2.2.1]heptan-2-yl)isoindoline-1,3-dione;
2-(2,6-dioxopiperidin-3-yl)-5-fluoro-6-((1R,4R)-5-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-2,5-diazabicyclo[2.2.1]heptan-2-yl)isoindoline-1,3-dione;
2-(2,6-dioxopiperidin-3-yl)-5-(5-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-2,5-diazabicyclo[2.2.2]octan-2-yl)isoindoline-1,3-dione;
2-(2,6-dioxopiperidin-3-yl)-5-fluoro-6-(5-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-2,5-diazabicyclo[2.2.2]octan-2-yl)isoindoline-1,3-dione;
2-(2,6-dioxopiperidin-3-yl)-5-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperazin-1-yl-2,2,3,3,5,5,6,6-d₈)isoindoline-1,3-dione;
2-(2,6-dioxopiperidin-3-yl)-5-fluoro-6-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperazin-1-yl-2,2,3,3,5,5,6,6-d₈)isoindoline-1,3-dione;
2-(2,6-dioxopiperidin-3-yl)-4-fluoro-5-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperazin-1-yl-2,2,3,3,5,5,6,6-d₈)isoindoline-1,3-dione;
2-(2,6-dioxopiperidin-3-yl)-4-fluoro-6-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperazin-1-yl-2,2,3,3,5,5,6,6-d₈)isoindoline-1,3-dione;
2-(2,6-dioxopiperidin-3-yl)-4,5-difluoro-6-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperazin-1-yl-2,2,3,3,5,5,6,6-d₈)isoindoline-1,3-dione;
2-(2,6-dioxopiperidin-3-yl)-4,7-difluoro-5-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperazin-1-yl-2,2,3,3,5,5,6,6-d₈)isoindoline-1,3-dione;
2-(2,6-dioxopiperidin-3-yl)-4,6-difluoro-5-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperazin-1-yl-2,2,3,3,5,5,6,6-d₈)isoindoline-1,3-dione;
2-(2,6-dioxopiperidin-3-yl)-4,5,7-trifluoro-6-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperazin-1-yl-2,2,3,3,5,5,6,6-d₈)isoindoline-1,3-dione;
2-(2,6-dioxopiperidin-3-yl)-5-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-2,6-dimethylpiperazin-1-yl)isoindoline-1,3-dione;
2-(2,6-dioxopiperidin-3-yl)-5-fluoro-6-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-2,6-dimethylpiperazin-1-yl)isoindoline-1,3-dione;
2-(2,6-dioxopiperidin-3-yl)-5-fluoro-6-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-3,5-dimethylpiperazin-1-yl)isoindoline-1,3-dione;
2-(2,6-dioxopiperidin-3-yl)-5-(4-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)-3,5-dimethylpiperazin-1-yl)isoindoline-1,3-dione;
2-(2,6-dioxopiperidin-3-yl)-5-(1-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperidin-4-yl)isoindoline-1,3-dione;
2-(2,6-dioxopiperidin-3-yl)-5-fluoro-6-(1-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperidin-4-yl)isoindoline-1,3-dione;
2-(2,6-dioxopiperidin-3-yl)-5-((R)-3-(((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)amino)piperidin-1-yl)isoindoline-1,3-dione;
2-(2,6-dioxopiperidin-3-yl)-5-((S)-3-(((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)amino)piperidin-1-yl)isoindoline-1,3-dione;
2-(2,6-dioxopiperidin-3-yl)-5-(((R)-1-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperidin-3-yl)amino)isoindoline-1,3-dione;
2-(2,6-dioxopiperidin-3-yl)-5-(((S)-1-((1-((6-((5-fluoro-4-(4-fluoro-1-isopropyl-2-methyl-1H-benzo[d]imidazol-6-yl)pyrimidin-2-yl)amino)pyridin-3-yl)methyl)piperidin-4-yl)methyl)piperidin-3-yl)amino)isoindoline-1,3-dione;
7-cyclopentyl-2-((5-(4-((4-(2-(2,6-dioxopiperidin-3-yl)-1,3-dioxoisoindolin-5-yl)piperazin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((4-(2-(2,6-dioxopiperidin-3-yl)-6-fluoro-1,3-dioxoisoindolin-5-yl)piperazin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-(((1R,4R)-5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-(((1S,4S)-5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-(((1S,4S)-5-(2-(2,6-dioxopiperidin-3-yl)-1,3-dioxoisoindolin-5-yl)-2,5-diazabicyclo[2.2.1]heptan-2-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-(((1R,4R)-5-(2-(2,6-dioxopiperidin-3-yl)-1,3-dioxoisoindolin-5-yl)-2,5-diazabicyclo[2.2.1]heptan-2-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)pyridin-2-yl)amino)-N,N-dimethyl-7H-pyrrolo [2,3-d]pyrimidine-6-carboxamide;
7-cyclopentyl-2-((5-(4-((4-(2-(2,6-dioxopiperidin-3-yl)-1,3-dioxoisoindolin-5-yl)-3,5-dimethylpiperazin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((4-(2-(2,6-dioxopiperidin-3-yl)-1,3-dioxoisoindolin-5-yl)-2,6-dimethylpiperazin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((4-(2-(2,6-dioxopiperidin-3-yl)-1,3-dioxoisoindolin-5-yl)piperidin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((4-(2-(2,6-dioxopiperidin-3-yl)-6-fluoro-1,3-dioxoisoindolin-5-yl)piperidin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((((3R)-1-(2-(2,6-dioxopiperidin-3-yl)-1,3-dioxoisoindolin-5-yl)piperidin-3-yl)amino)methyl)piperidin-1-yl)pyridin-2-yl)amino)-N,N-dimethyl-7H-pyrrolo[2,3-d]pyrimidine-6-carboxamide;
7-cyclopentyl-2-((5-(4-((((3S)-1-(2-(2,6-dioxopiperidin-3-yl)-1,3-dioxoisoindolin-5-yl)piperidin-3-yl)amino)methyl)piperidin-1-yl)pyridin-2-yl)amino)-N,N-dimethyl-7H-pyrrolo[2,3-d]pyrimidine-6-carboxamide;
7-cyclopentyl-2-((5-(4-(((3S)-3-((2-(2,6-dioxopiperidin-3-yl)-1,3-dioxoisoindolin-5-yl)amino)piperidin-1-yl)methyl)piperidin-1-yl)pyridin-2-yl)amino)-N,N-dimethyl-7H-pyrrolo[2,3-d]pyrimidine-6-carboxamide;
7-cyclopentyl-2-((5-(4-(((3R)-3-((2-(2,6-dioxopiperidin-3-yl)-1,3-dioxoisoindolin-5-yl)amino)piperidin-1-yl)methyl)piperidin-1-yl)pyridin-2-yl)amino)-N,N-dimethyl-7H-pyrrolo[2,3-d]pyrimidine-6-carboxamide;
5-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperazin-1-yl)-2-(2,6-dioxopiperidin-3-yl)isoindoline-1,3-dione;
5-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperazin-1-yl)-2-(2,6-dioxopiperidin-3-yl)-6-fluoroisoindoline-1,3-dione;
5-(3-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-3,6-diazabicyclo[3.1.1]heptan-6-yl)-2-(2,6-dioxopiperidin-3-yl)-6-fluoroisoindoline-1,3-dione;
5-(3-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-3,6-diazabicyclo[3.1.1]heptan-6-yl)-2-(2,6-dioxopiperidin-3-yl)isoindoline-1,3-dione;
5-(6-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-3,6-diazabicyclo[3.1.1]heptan-3-yl)-2-(2,6-dioxopiperidin-3-yl)isoindoline-1,3-dione;
5-(6-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-3,6-diazabicyclo[3.1.1]heptan-3-yl)-2-(2,6-dioxopiperidin-3-yl)-6-fluoroisoindoline-1,3-dione;
5-(3-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-3,8-diazabicyclo[3.2.1]octan-8-yl)-2-(2,6-dioxopiperidin-3-yl)-6-fluoroisoindoline-1,3-dione;
5-(3-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-3,8-diazabicyclo[3.2.1]octan-8-yl)-2-(2,6-dioxopiperidin-3-yl)isoindoline-1,3-dione;
5-(8-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-3,8-diazabicyclo[3.2.1]octan-3-yl)-2-(2,6-dioxopiperidin-3-yl)isoindoline-1,3-dione;
5-(8-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-3,8-diazabicyclo[3.2.1]octan-3-yl)-2-(2,6-dioxopiperidin-3-yl)-6-fluoroisoindoline-1,3-dione;
5-(5-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-2,5-diazabicyclo[2.2.1]heptan-2-yl)-2-(2,6-dioxopiperidin-3-yl)-6-fluoroisoindoline-1,3-dione;
5-(5-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-2,5-diazabicyclo[2.2.1]heptan-2-yl)-2-(2,6-dioxopiperidin-3-yl)isoindoline-1,3-dione;
5-(5-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-2,5-diazabicyclo[2.2.2]octan-2-yl)-2-(2,6-dioxopiperidin-3-yl)isoindoline-1,3-dione;
5-(5-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-2,5-diazabicyclo[2.2.2]octan-2-yl)-2-(2,6-dioxopiperidin-3-yl)-6-fluoroisoindoline-1,3-dione;
5-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperazin-1-yl-2,2,3,3,5,5,6,6-d₈)-2-(2,6-dioxopiperidin-3-yl)isoindoline-1,3-dione;
5-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperazin-1-yl-2,2,3,3,5,5,6,6-d₈)-2-(2,6-dioxopiperidin-3-yl)-6-fluoroisoindoline-1,3-dione;
6-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
5-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
6-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
5-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-2,6-dimethylpiperazin-1-yl)-2-(2,6-dioxopiperidin-3-yl)isoindoline-1,3-dione;
5-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-2,6-dimethylpiperazin-1-yl)-2-(2,6-dioxopiperidin-3-yl)-6-fluoroisoindoline-1,3-dione;
5-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-3,5-dimethylpiperazin-1-yl)-2-(2,6-dioxopiperidin-3-yl)-6-fluoroisoindoline-1,3-dione;
5-(4-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)-3,5-dimethylpiperazin-1-yl)-2-(2,6-dioxopiperidin-3-yl)isoindoline-1,3-dione;
5-(1-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperidin-4-yl)-2-(2,6-dioxopiperidin-3-yl)isoindoline-1,3-dione;
5-(1-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperidin-4-yl)-2-(2,6-dioxopiperidin-3-yl)-6-fluoroisoindoline-1,3-dione;
5-((R)-3-(((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)amino)piperidin-1-yl)-2-(2,6-dioxopiperidin-3-yl)isoindoline-1,3-dione;
5-((S)-3-(((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)amino)piperidin-1-yl)-2-(2,6-dioxopiperidin-3-yl)isoindoline-1,3-dione;
5-(((S)-1-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperidin-3-yl)amino)-2-(2,6-dioxopiperidin-3-yl)isoindoline-1,3-dione;
5-(((R)-1-((1-(6-((6-acetyl-8-cyclopentyl-5-methyl-7-oxo-7,8-dihydropyrido[2,3-d]pyrimidin-2-yl)amino)pyridin-3-yl)piperidin-4-yl)methyl)piperidin-3-yl)amino)-2-(2,6-dioxopiperidin-3-yl)isoindoline-1,3-dione;
5-(4-((1-(4-((5-chloro-4-((2-(dimethylphosphoryl)phenyl)amino)pyrimidin-2-yl)amino)-3-methoxyphenyl)piperidin-4-yl)methyl)piperazin-1-yl)-2-(2,6-dioxopiperidin-3-yl)isoindoline-1,3-dione;
5-(4-((1-(4-((5-chloro-4-((2-(dimethylphosphoryl)phenyl)amino)pyrimidin-2-yl)amino)-3-methoxyphenyl)piperidin-4-yl)methyl)piperazin-1-yl)-2-(2,6-dioxopiperidin-3-yl)-6-fluoroisoindoline-1,3-dione;
5-(3-((1-(4-((5-chloro-4-((2-(dimethylphosphoryl)phenyl)amino)pyrimidin-2-yl)amino)-3-methoxyphenyl)piperidin-4-yl)methyl)-3,6-diazabicyclo[3.1.1]heptan-6-yl)-2-(2,6-dioxopiperidin-3-yl)-6-fluoroisoindoline-1,3-dione;
5-(3-((1-(4-((5-chloro-4-((2-(dimethylphosphoryl)phenyl)amino)pyrimidin-2-yl)amino)-3-methoxyphenyl)piperidin-4-yl)methyl)-3,6-diazabicyclo[3.1.1]heptan-6-yl)-2-(2,6-dioxopiperidin-3-yl)isoindoline-1,3-dione;
5-(6-((1-(4-((5-chloro-4-((2-(dimethylphosphoryl)phenyl)amino)pyrimidin-2-yl)amino)-3-methoxyphenyl)piperidin-4-yl)methyl)-3,6-diazabicyclo[3.1.1]heptan-3-yl)-2-(2,6-dioxopiperidin-3-yl)isoindoline-1,3-dione;
5-(6-((1-(4-((5-chloro-4-((2-(dimethylphosphoryl)phenyl)amino)pyrimidin-2-yl)amino)-3-methoxyphenyl)piperidin-4-yl)methyl)-3,6-diazabicyclo[3.1.1]heptan-3-yl)-2-(2,6-dioxopiperidin-3-yl)-6-fluoroisoindoline-1,3-dione;
5-(3-((1-(4-((5-chloro-4-((2-(dimethylphosphoryl)phenyl)amino)pyrimidin-2-yl)amino)-3-methoxyphenyl)piperidin-4-yl)methyl)-3,8-diazabicyclo[3.2.1]octan-8-yl)-2-(2,6-dioxopiperidin-3-yl)-6-fluoroisoindoline-1,3-dione;
5-(3-((1-(4-((5-chloro-4-((2-(dimethylphosphoryl)phenyl)amino)pyrimidin-2-yl)amino)-3-methoxyphenyl)piperidin-4-yl)methyl)-3,8-diazabicyclo[3.2.1]octan-8-yl)-2-(2,6-dioxopiperidin-3-yl)isoindoline-1,3-dione;
5-(8-((1-(4-((5-chloro-4-((2-(dimethylphosphoryl)phenyl)amino)pyrimidin-2-yl)amino)-3-methoxyphenyl)piperidin-4-yl)methyl)-3,8-diazabicyclo[3.2.1]octan-3-yl)-2-(2,6-dioxopiperidin-3-yl)isoindoline-1,3-dione;
5-(8-((1-(4-((5-chloro-4-((2-(dimethylphosphoryl)phenyl)amino)pyrimidin-2-yl)amino)-3-methoxyphenyl)piperidin-4-yl)methyl)-3,8-diazabicyclo[3.2.1]octan-3-yl)-2-(2,6-dioxopiperidin-3-yl)-6-fluoroisoindoline-1,3-dione;
5-(5-((1-(4-((5-chloro-4-((2-(dimethylphosphoryl)phenyl)amino)pyrimidin-2-yl)amino)-3-methoxyphenyl)piperidin-4-yl)methyl)-2,5-diazabicyclo[2.2.1]heptan-2-yl)-2-(2,6-dioxopiperidin-3-yl)-6-fluoroisoindoline-1,3-dione;
5-(5-((1-(4-((5-chloro-4-((2-(dimethylphosphoryl)phenyl)amino)pyrimidin-2-yl)amino)-3-methoxyphenyl)piperidin-4-yl)methyl)-2,5-diazabicyclo[2.2.1]heptan-2-yl)-2-(2,6-dioxopiperidin-3-yl)isoindoline-1,3-dione;
5-(5-((1-(4-((5-chloro-4-((2-(dimethylphosphoryl)phenyl)amino)pyrimidin-2-yl)amino)-3-methoxyphenyl)piperidin-4-yl)methyl)-2,5-diazabicyclo[2.2.2]octan-2-yl)-2-(2,6-dioxopiperidin-3-yl)isoindoline-1,3-dione;
5-(5-((1-(4-((5-chloro-4-((2-(dimethylphosphoryl)phenyl)amino)pyrimidin-2-yl)amino)-3-methoxyphenyl)piperidin-4-yl)methyl)-2,5-diazabicyclo[2.2.2]octan-2-yl)-2-(2,6-dioxopiperidin-3-yl)-6-fluoroisoindoline-1,3-dione;
5-(4-((1-(4-((5-chloro-4-((2-(dimethylphosphoryl)phenyl)amino)pyrimidin-2-yl)amino)-3-methoxyphenyl)piperidin-4-yl)methyl)piperazin-1-yl-2,2,3,3,5,5,6,6-d₈)-2-(2,6-dioxopiperidin-3-yl)isoindoline-1,3-dione;
5-(4-((1-(4-((5-chloro-4-((2-(dimethylphosphoryl)phenyl)amino)pyrimidin-2-yl)amino)-3-methoxyphenyl)piperidin-4-yl)methyl)piperazin-1-yl-2,2,3,3,5,5,6,6-d₈)-2-(2,6-dioxopiperidin-3-yl)-6-fluoroisoindoline-1,3-dione;
6-(4-((1-(4-((5-chloro-4-((2-(dimethylphosphoryl)phenyl)amino)pyrimidin-2-yl)amino)-3-methoxyphenyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(4-((5-chloro-4-((2-(dimethylphosphoryl)phenyl)amino)pyrimidin-2-yl)amino)-3-methoxyphenyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(4-((5-chloro-4-((2-(dimethylphosphoryl)phenyl)amino)pyrimidin-2-yl)amino)-3-methoxyphenyl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
5-(4-((1-(4-((5-chloro-4-((2-(dimethylphosphoryl)phenyl)amino)pyrimidin-2-yl)amino)-3-methoxyphenyl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
6-(4-((1-(4-((5-chloro-4-((2-(dimethylphosphoryl)phenyl)amino)pyrimidin-2-yl)amino)-3-methoxyphenyl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
5-(4-((1-(4-((5-chloro-4-((2-(dimethylphosphoryl)phenyl)amino)pyrimidin-2-yl)amino)-3-methoxyphenyl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((1-(4-((5-chloro-4-((2-(dimethylphosphoryl)phenyl)amino)pyrimidin-2-yl)amino)-3-methoxyphenyl)piperidin-4-yl)methyl)-2,6-dimethylpiperazin-1-yl)-2-(2,6-dioxopiperidin-3-yl)isoindoline-1,3-dione;
5-(4-((1-(4-((5-chloro-4-((2-(dimethylphosphoryl)phenyl)amino)pyrimidin-2-yl)amino)-3-methoxyphenyl)piperidin-4-yl)methyl)-2,6-dimethylpiperazin-1-yl)-2-(2,6-dioxopiperidin-3-yl)-6-fluoroisoindoline-1,3-dione;
5-(4-((1-(4-((5-chloro-4-((2-(dimethylphosphoryl)phenyl)amino)pyrimidin-2-yl)amino)-3-methoxyphenyl)piperidin-4-yl)methyl)-3,5-dimethylpiperazin-1-yl)-2-(2,6-dioxopiperidin-3-yl)isoindoline-1,3-dione;
5-(4-((1-(4-((5-chloro-4-((2-(dimethylphosphoryl)phenyl)amino)pyrimidin-2-yl)amino)-3-methoxyphenyl)piperidin-4-yl)methyl)-3,5-dimethylpiperazin-1-yl)-2-(2,6-dioxopiperidin-3-yl)-6-fluoroisoindoline-1,3-dione;
5-(1-((1-(4-((5-chloro-4-((2-(dimethylphosphoryl)phenyl)amino)pyrimidin-2-yl)amino)-3-methoxyphenyl)piperidin-4-yl)methyl)piperidin-4-yl)-2-(2,6-dioxopiperidin-3-yl)-6-fluoroisoindoline-1,3-dione;
5-(1-((1-(4-((5-chloro-4-((2-(dimethylphosphoryl)phenyl)amino)pyrimidin-2-yl)amino)-3-methoxyphenyl)piperidin-4-yl)methyl)piperidin-4-yl)-2-(2,6-dioxopiperidin-3-yl)isoindoline-1,3-dione;
5-((R)-3-(((1-(4-((5-chloro-4-((2-(dimethylphosphoryl)phenyl)amino)pyrimidin-2-yl)amino)-3-methoxyphenyl)piperidin-4-yl)methyl)amino)piperidin-1-yl)-2-(2,6-dioxopiperidin-3-yl)isoindoline-1,3-dione;
5-((S)-3-(((1-(4-((5-chloro-4-((2-(dimethylphosphoryl)phenyl)amino)pyrimidin-2-yl)amino)-3-methoxyphenyl)piperidin-4-yl)methyl)amino)piperidin-1-yl)-2-(2,6-dioxopiperidin-3-yl)isoindoline-1,3-dione;
5-(((S)-1-((1-(4-((5-chloro-4-((2-(dimethylphosphoryl)phenyl)amino)pyrimidin-2-yl)amino)-3-methoxyphenyl)piperidin-4-yl)methyl)piperidin-3-yl)amino)-2-(2,6-dioxopiperidin-3-yl)isoindoline-1,3-dione;
5-(((R)-1-((1-(4-((5-chloro-4-((2-(dimethylphosphoryl)phenyl)amino)pyrimidin-2-yl)amino)-3-methoxyphenyl)piperidin-4-yl)methyl)piperidin-3-yl)amino)-2-(2,6-dioxopiperidin-3-yl)isoindoline-1,3-dione;
5-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)piperazin-1-yl)-2-(2,6-dioxopiperidin-3-yl)isoindoline-1,3-dione;
5-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)piperazin-1-yl)-2-(2,6-dioxopiperidin-3-yl)-6-fluoroisoindoline-1,3-dione;
5-(3-((1-(4-((5-chloro-4-((2-(isopropylsulfonyl)phenyl)amino)pyrimidin-2-yl)amino)-5-isopropoxy-2-methylphenyl)piperidin-4-yl)methyl)-3,6-diazabicyclo[3.1.1]heptan-6-yl)-2-(2,6-dioxopiperidin-3-yl)-6-fluoroisoindoline-1,3-dione;
5-(3-((1-(4-((5-chloro-4-((2-(isopropylsulfonyl)phenyl)amino)pyrimidin-2-yl)amino)-5-isopropoxy-2-methylphenyl)piperidin-4-yl)methyl)-3,6-diazabicyclo[3.1.1]heptan-6-yl)-2-(2,6-dioxopiperidin-3-yl)isoindoline-1,3-dione;
5-(6-((1-(4-((5-chloro-4-((2-(isopropylsulfonyl)phenyl)amino)pyrimidin-2-yl)amino)-5-isopropoxy-2-methylphenyl)piperidin-4-yl)methyl)-3,6-diazabicyclo[3.1.1]heptan-3-yl)-2-(2,6-dioxopiperidin-3-yl)isoindoline-1,3-dione;
5-(6-((1-(4-((5-chloro-4-((2-(isopropylsulfonyl)phenyl)amino)pyrimidin-2-yl)amino)-5-isopropoxy-2-methylphenyl)piperidin-4-yl)methyl)-3,6-diazabicyclo[3.1.1]heptan-3-yl)-2-(2,6-dioxopiperidin-3-yl)-6-fluoroisoindoline-1,3-dione;
5-(3-((1-(4-((5-chloro-4-((2-(isopropylsulfonyl)phenyl)amino)pyrimidin-2-yl)amino)-5-isopropoxy-2-methylphenyl)piperidin-4-yl)methyl)-3,8-diazabicyclo[3.2.1]octan-8-yl)-2-(2,6-dioxopiperidin-3-yl)-6-fluoroisoindoline-1,3-dione;
5-(3-((1-(4-((5-chloro-4-((2-(isopropylsulfonyl)phenyl)amino)pyrimidin-2-yl)amino)-5-isopropoxy-2-methylphenyl)piperidin-4-yl)methyl)-3,8-diazabicyclo[3.2.1]octan-8-yl)-2-(2,6-dioxopiperidin-3-yl)isoindoline-1,3-dione;
5-(8-((1-(4-((5-chloro-4-((2-(isopropylsulfonyl)phenyl)amino)pyrimidin-2-yl)amino)-5-isopropoxy-2-methylphenyl)piperidin-4-yl)methyl)-3,8-diazabicyclo[3.2.1]octan-3-yl)-2-(2,6-dioxopiperidin-3-yl)isoindoline-1,3-dione;
5-(8-((1-(4-((5-chloro-4-((2-(isopropylsulfonyl)phenyl)amino)pyrimidin-2-yl)amino)-5-isopropoxy-2-methylphenyl)piperidin-4-yl)methyl)-3,8-diazabicyclo[3.2.1]octan-3-yl)-2-(2,6-dioxopiperidin-3-yl)-6-fluoroisoindoline-1,3-dione;
5-(5-((1-(4-((5-chloro-4-((2-(isopropylsulfonyl)phenyl)amino)pyrimidin-2-yl)amino)-5-isopropoxy-2-methylphenyl)piperidin-4-yl)methyl)-2,5-diazabicyclo[2.2.1]heptan-2-yl)-2-(2,6-dioxopiperidin-3-yl)isoindoline-1,3-dione;
5-(5-((1-(4-((5-chloro-4-((2-(isopropylsulfonyl)phenyl)amino)pyrimidin-2-yl)amino)-5-isopropoxy-2-methylphenyl)piperidin-4-yl)methyl)-2,5-diazabicyclo[2.2.1]heptan-2-yl)-2-(2,6-dioxopiperidin-3-yl)-6-fluoroisoindoline-1,3-dione;
5-(5-((1-(4-((5-chloro-4-((2-(isopropylsulfonyl)phenyl)amino)pyrimidin-2-yl)amino)-5-isopropoxy-2-methylphenyl)piperidin-4-yl)methyl)-2,5-diazabicyclo[2.2.2]octan-2-yl)-2-(2,6-dioxopiperidin-3-yl)isoindoline-1,3-dione;
5-(5-((1-(4-((5-chloro-4-((2-(isopropylsulfonyl)phenyl)amino)pyrimidin-2-yl)amino)-5-isopropoxy-2-methylphenyl)piperidin-4-yl)methyl)-2,5-diazabicyclo[2.2.2]octan-2-yl)-2-(2,6-dioxopiperidin-3-yl)-6-fluoroisoindoline-1,3-dione;
5-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)piperazin-1-yl-2,2,3,3,5,5,6,6-d₈)-2-(2,6-dioxopiperidin-3-yl)isoindoline-1,3-dione;
5-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)piperazin-1-yl-2,2,3,3,5,5,6,6-d₈)-2-(2,6-dioxopiperidin-3-yl)-6-fluoroisoindoline-1,3-dione;
6-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
5-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
5-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
6-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
5-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)-2,6-dimethylpiperazin-1-yl)-2-(2,6-dioxopiperidin-3-yl)isoindoline-1,3-dione;
5-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)-2,6-dimethylpiperazin-1-yl)-2-(2,6-dioxopiperidin-3-yl)-6-fluoroisoindoline-1,3-dione;
5-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)-3,5-dimethylpiperazin-1-yl)-2-(2,6-dioxopiperidin-3-yl)isoindoline-1,3-dione;
5-(4-((1-(4-((5-chloro-4-((2-(isopropylsulfonyl)phenyl)amino)pyrimidin-2-yl)amino)-5-isopropoxy-2-methylphenyl)piperidin-4-yl)methyl)-3,5-dimethylpiperazin-1-yl)-2-(2,6-dioxopiperidin-3-yl)-6-fluoroisoindoline-1,3-dione;
5-(1-((1-(4-((5-chloro-4-((2-(isopropylsulfonyl)phenyl)amino)pyrimidin-2-yl)amino)-5-isopropoxy-2-methylphenyl)piperidin-4-yl)methyl)piperidin-4-yl)-2-(2,6-dioxopiperidin-3-yl)isoindoline-1,3-dione;
5-(1-((1-(4-((5-chloro-4-((2-(isopropylsulfonyl)phenyl)amino)pyrimidin-2-yl)amino)-5-isopropoxy-2-methylphenyl)piperidin-4-yl)methyl)piperidin-4-yl)-2-(2,6-dioxopiperidin-3-yl)-6-fluoroisoindoline-1,3-dione;
5-((R)-3-(((1-(4-((5-chloro-4-((2-(isopropylsulfonyl)phenyl)amino)pyrimidin-2-yl)amino)-5-isopropoxy-2-methylphenyl)piperidin-4-yl)methyl)amino)piperidin-1-yl)-2-(2,6-dioxopiperidin-3-yl)isoindoline-1,3-dione;
5-((S)-3-(((1-(4-((5-chloro-4-((2-(isopropylsulfonyl)phenyl)amino)pyrimidin-2-yl)amino)-5-isopropoxy-2-methylphenyl)piperidin-4-yl)methyl)amino)piperidin-1-yl)-2-(2,6-dioxopiperidin-3-yl)isoindoline-1,3-dione;
5-(((S)-1-((1-(4-((5-chloro-4-((2-(isopropylsulfonyl)phenyl)amino)pyrimidin-2-yl)amino)-5-isopropoxy-2-methylphenyl)piperidin-4-yl)methyl)piperidin-3-yl)amino)-2-(2,6-dioxopiperidin-3-yl)isoindoline-1,3-dione;
5-(((R)-1-((1-(4-((5-chloro-4-((2-(isopropylsulfonyl)phenyl)amino)pyrimidin-2-yl)amino)-5-isopropoxy-2-methylphenyl)piperidin-4-yl)methyl)piperidin-3-yl)amino)-2-(2,6-dioxopiperidin-3-yl)isoindoline-1,3-dione;
8-(4-((4-(2-(2,6-dioxopiperidin-3-yl)-1,3-dioxoisoindolin-5-yl)piperazin-1-yl)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-6-fluoro-1,3-dioxoisoindolin-5-yl)piperazin-1-yl)methyl)piperidin-1-yl)-9-ethyl-6,6-dimethyl-11-oxo-6,11-dihydro-5H-benzo[b]carbazole-3-carbonitrile;
8-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)-9-ethyl-6,6-dimethyl-11-oxo-6,11-dihydro-5H-benzo[b]carbazole-3-carbonitrile;
8-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)-9-ethyl-6,6-dimethyl-11-oxo-6,11-dihydro-5H-benzo[b]carbazole-3-carbonitrile;
8-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)-9-ethyl-6,6-dimethyl-11-oxo-6,11-dihydro-5H-benzo[b]carbazole-3-carbonitrile;
8-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)-9-ethyl-6,6-dimethyl-11-oxo-6,11-dihydro-5H-benzo[b]carbazole-3-carbonitrile;
8-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)-9-ethyl-6,6-dimethyl-11-oxo-6,11-dihydro-5H-benzo[b]carbazole-3-carbonitrile;
8-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)-9-ethyl-6,6-dimethyl-11-oxo-6,11-dihydro-5H-benzo[b]carbazole-3-carbonitrile;
8-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)-9-ethyl-6,6-dimethyl-11-oxo-6,11-dihydro-5H-benzo[b]carbazole-3-carbonitrile;
8-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)-9-ethyl-6,6-dimethyl-11-oxo-6,11-dihydro-5H-benzo[b]carbazole-3-carbonitrile;
8-(4-((5-(2-(2,6-dioxopiperidin-3-yl)-1,3-dioxoisoindolin-5-yl)-2,5-diazabicyclo[2.2.1]heptan-2-yl)methyl)piperidin-1-yl)-9-ethyl-6,6-dimethyl-11-oxo-6,11-dihydro-5H-benzo[b]carbazole-3-carbonitrile;
8-(4-((5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)-9-ethyl-6,6-dimethyl-11-oxo-6,11-dihydro-5H-benzo[b]carbazole-3-carbonitrile;
8-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)-9-ethyl-6,6-dimethyl-11-oxo-6,11-dihydro-5H-benzo[b]carbazole-3-carbonitrile;
8-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-1,3-dioxoisoindolin-5-yl)-3,5-dimethylpiperazin-1-yl)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-1,3-dioxoisoindolin-5-yl)-2,6-dimethylpiperazin-1-yl)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-6-fluoro-1,3-dioxoisoindolin-5-yl)piperidin-1-yl)methyl)piperidin-1-yl)-9-ethyl-6,6-dimethyl-11-oxo-6,11-dihydro-5H-benzo[b]carbazole-3-carbonitrile;
8-(4-((4-(2-(2,6-dioxopiperidin-3-yl)-1,3-dioxoisoindolin-5-yl)piperidin-1-yl)methyl)piperidin-1-yl)-9-ethyl-6,6-dimethyl-11-oxo-6,11-dihydro-5H-benzo[b]carbazole-3-carbonitrile;
5-(4-((4-(4-(6-amino-5-((R)-1-(2,6-dichloro-3-fluorophenyl)ethoxy)pyridin-3-yl)-1H-pyrazol-1-yl)cyclohexyl)methyl)piperazin-1-yl)-2-(2,6-dioxopiperidin-3-yl)isoindoline-1,3-dione;
5-(4-((4-(4-(6-amino-5-((R)-1-(2,6-dichloro-3-fluorophenyl)ethoxy)pyridin-3-yl)-1H-pyrazol-1-yl)cyclohexyl)methyl)piperazin-1-yl)-2-(2,6-dioxopiperidin-3-yl)-6-fluoroisoindoline-1,3-dione;
5-(3-((4-(4-(6-amino-5-((R)-1-(2,6-dichloro-3-fluorophenyl)ethoxy)pyridin-3-yl)-1H-pyrazol-1-yl)cyclohexyl)methyl)-3,6-diazabicyclo[3.1.1]heptan-6-yl)-2-(2,6-dioxopiperidin-3-yl)isoindoline-1,3-dione;
5-(3-((4-(4-(6-amino-5-((R)-1-(2,6-dichloro-3-fluorophenyl)ethoxy)pyridin-3-yl)-1H-pyrazol-1-yl)cyclohexyl)methyl)-3,6-diazabicyclo[3.1.1]heptan-6-yl)-2-(2,6-dioxopiperidin-3-yl)-6-fluoroisoindoline-1,3-dione;
5-(6-((4-(4-(6-amino-5-((R)-1-(2,6-dichloro-3-fluorophenyl)ethoxy)pyridin-3-yl)-1H-pyrazol-1-yl)cyclohexyl)methyl)-3,6-diazabicyclo[3.1.1]heptan-3-yl)-2-(2,6-dioxopiperidin-3-yl)isoindoline-1,3-dione;
5-(6-((4-(4-(6-amino-5-((R)-1-(2,6-dichloro-3-fluorophenyl)ethoxy)pyridin-3-yl)-1H-pyrazol-1-yl)cyclohexyl)methyl)-3,6-diazabicyclo[3.1.1]heptan-3-yl)-2-(2,6-dioxopiperidin-3-yl)-6-fluoroisoindoline-1,3-dione;
5-(3-((4-(4-(6-amino-5-((R)-1-(2,6-dichloro-3-fluorophenyl)ethoxy)pyridin-3-yl)-1H-pyrazol-1-yl)cyclohexyl)methyl)-3,8-diazabicyclo[3.2.1]octan-8-yl)-2-(2,6-dioxopiperidin-3-yl)isoindoline-1,3-dione;
5-(3-((4-(4-(6-amino-5-((R)-1-(2,6-dichloro-3-fluorophenyl)ethoxy)pyridin-3-yl)-1H-pyrazol-1-yl)cyclohexyl)methyl)-3,8-diazabicyclo[3.2.1]octan-8-yl)-2-(2,6-dioxopiperidin-3-yl)-6-fluoroisoindoline-1,3-dione;
5-(8-((4-(4-(6-amino-5-((R)-1-(2,6-dichloro-3-fluorophenyl)ethoxy)pyridin-3-yl)-1H-pyrazol-1-yl)cyclohexyl)methyl)-3,8-diazabicyclo[3.2.1]octan-3-yl)-2-(2,6-dioxopiperidin-3-yl)isoindoline-1,3-dione;
5-(8-((4-(4-(6-amino-5-((R)-1-(2,6-dichloro-3-fluorophenyl)ethoxy)pyridin-3-yl)-1H-pyrazol-1-yl)cyclohexyl)methyl)-3,8-diazabicyclo[3.2.1]octan-3-yl)-2-(2,6-dioxopiperidin-3-yl)-6-fluoroisoindoline-1,3-dione;
5-(5-((4-(4-(6-amino-5-((R)-1-(2,6-dichloro-3-fluorophenyl)ethoxy)pyridin-3-yl)-1H-pyrazol-1-yl)cyclohexyl)methyl)-2,5-diazabicyclo[2.2.1]heptan-2-yl)-2-(2,6-dioxopiperidin-3-yl)isoindoline-1,3-dione;
5-(5-((4-(4-(6-amino-5-((R)-1-(2,6-dichloro-3-fluorophenyl)ethoxy)pyridin-3-yl)-1H-pyrazol-1-yl)cyclohexyl)methyl)-2,5-diazabicyclo[2.2.1]heptan-2-yl)-2-(2,6-dioxopiperidin-3-yl)-6-fluoroisoindoline-1,3-dione;
5-(4-((4-(4-(6-amino-5-((R)-1-(2,6-dichloro-3-fluorophenyl)ethoxy)pyridin-3-yl)-1H-pyrazol-1-yl)cyclohexyl)methyl)piperazin-1-yl)-2-(2,6-dioxopiperidin-3-yl)isoindoline-1,3-dione;
5-(5-((4-(4-(6-amino-5-((R)-1-(2,6-dichloro-3-fluorophenyl)ethoxy)pyridin-3-yl)-1H-pyrazol-1-yl)cyclohexyl)methyl)-2,5-diazabicyclo[2.2.2]octan-2-yl)-2-(2,6-dioxopiperidin-3-yl)-6-fluoroisoindoline-1,3-dione;
5-(4-((4-(4-(6-amino-5-((R)-1-(2,6-dichloro-3-fluorophenyl)ethoxy)pyridin-3-yl)-1H-pyrazol-1-yl)cyclohexyl)methyl)piperazin-1-yl-2,2,3,3,5,5,6,6-d₈)-2-(2,6-dioxopiperidin-3-yl)isoindoline-1,3-dione;
5-(4-((4-(4-(6-amino-5-((R)-1-(2,6-dichloro-3-fluorophenyl)ethoxy)pyridin-3-yl)-1H-pyrazol-1-yl)cyclohexyl)methyl)piperazin-1-yl-2,2,3,3,5,5,6,6-d₈)-2-(2,6-dioxopiperidin-3-yl)-6-fluoroisoindoline-1,3-dione;
5-(4-((4-(4-(6-amino-5-((R)-1-(2,6-dichloro-3-fluorophenyl)ethoxy)pyridin-3-yl)-1H-pyrazol-1-yl)cyclohexyl)methyl)piperazin-1-yl-2,2,3,3,5,5,6,6-d₈)-2-(2,6-dioxopiperidin-3-yl)-4-fluoroisoindoline-1,3-dione;
6-(4-((4-(4-(6-amino-5-((R)-1-(2,6-dichloro-3-fluorophenyl)ethoxy)pyridin-3-yl)-1H-pyrazol-1-yl)cyclohexyl)methyl)piperazin-1-yl-2,2,3,3,5,5,6,6-d₈)-2-(2,6-dioxopiperidin-3-yl)-4-fluoroisoindoline-1,3-dione;
6-(4-((4-(4-(6-amino-5-((R)-1-(2,6-dichloro-3-fluorophenyl)ethoxy)pyridin-3-yl)-1H-pyrazol-1-yl)cyclohexyl)methyl)piperazin-1-yl-2,2,3,3,5,5,6,6-d₈)-2-(2,6-dioxopiperidin-3-yl)-4,5-difluoroisoindoline-1,3-dione;
5-(4-((4-(4-(6-amino-5-((R)-1-(2,6-dichloro-3-fluorophenyl)ethoxy)pyridin-3-yl)-1H-pyrazol-1-yl)cyclohexyl)methyl)piperazin-1-yl-2,2,3,3,5,5,6,6-d₈)-2-(2,6-dioxopiperidin-3-yl)-4,7-difluoroisoindoline-1,3-dione;
5-(4-((4-(4-(6-amino-5-((R)-1-(2,6-dichloro-3-fluorophenyl)ethoxy)pyridin-3-yl)-1H-pyrazol-1-yl)cyclohexyl)methyl)piperazin-1-yl-2,2,3,3,5,5,6,6-d₈)-2-(2,6-dioxopiperidin-3-yl)-4,6-difluoroisoindoline-1,3-dione;
5-(4-((4-(4-(6-amino-5-((R)-1-(2,6-dichloro-3-fluorophenyl)ethoxy)pyridin-3-yl)-1H-pyrazol-1-yl)cyclohexyl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((4-(4-(6-amino-5-((R)-1-(2,6-dichloro-3-fluorophenyl)ethoxy)pyridin-3-yl)-1H-pyrazol-1-yl)cyclohexyl)methyl)-2,6-dimethylpiperazin-1-yl)-2-(2,6-dioxopiperidin-3-yl)isoindoline-1,3-dione;
5-(4-((4-(4-(6-amino-5-((R)-1-(2,6-dichloro-3-fluorophenyl)ethoxy)pyridin-3-yl)-1H-pyrazol-1-yl)cyclohexyl)methyl)-2,6-dimethylpiperazin-1-yl)-2-(2,6-dioxopiperidin-3-yl)-6-fluoroisoindoline-1,3-dione;
5-(4-((4-(4-(6-amino-5-((R)-1-(2,6-dichloro-3-fluorophenyl)ethoxy)pyridin-3-yl)-1H-pyrazol-1-yl)cyclohexyl)methyl)-3,5-dimethylpiperazin-1-yl)-2-(2,6-dioxopiperidin-3-yl)isoindoline-1,3-dione;
5-(4-((4-(4-(6-amino-5-((R)-1-(2,6-dichloro-3-fluorophenyl)ethoxy)pyridin-3-yl)-1H-pyrazol-1-yl)cyclohexyl)methyl)-3,5-dimethylpiperazin-1-yl)-2-(2,6-dioxopiperidin-3-yl)-6-fluoroisoindoline-1,3-dione;
5-(1-((4-(4-(6-amino-5-((R)-1-(2,6-dichloro-3-fluorophenyl)ethoxy)pyridin-3-yl)-1H-pyrazol-1-yl)cyclohexyl)methyl)piperidin-4-yl)-2-(2,6-dioxopiperidin-3-yl)isoindoline-1,3-dione;
5-(1-((4-(4-(6-amino-5-((R)-1-(2,6-dichloro-3-fluorophenyl)ethoxy)pyridin-3-yl)-1H-pyrazol-1-yl)cyclohexyl)methyl)piperidin-4-yl)-2-(2,6-dioxopiperidin-3-yl)-6-fluoroisoindoline-1,3-dione;
5-((R)-3-(((4-(4-(6-amino-5-((R)-1-(2,6-dichloro-3-fluorophenyl)ethoxy)pyridin-3-yl)-1H-pyrazol-1-yl)cyclohexyl)methyl)amino)piperidin-1-yl)-2-(2,6-dioxopiperidin-3-yl)isoindoline-1,3-dione;
5-((S)-3-(((4-(4-(6-amino-5-((R)-1-(2,6-dichloro-3-fluorophenyl)ethoxy)pyridin-3-yl)-1H-pyrazol-1-yl)cyclohexyl)methyl)amino)piperidin-1-yl)-2-(2,6-dioxopiperidin-3-yl)isoindoline-1,3-dione;
5-(((S)-1-((4-(4-(6-amino-5-((R)-1-(2,6-dichloro-3-fluorophenyl)ethoxy)pyridin-3-yl)-1H-pyrazol-1-yl)cyclohexyl)methyl)piperidin-3-yl)amino)-2-(2,6-dioxopiperidin-3-yl)isoindoline-1,3-dione;
5-(((R)-1-((4-(4-(6-amino-5-((R)-1-(2,6-dichloro-3-fluorophenyl)ethoxy)pyridin-3-yl)-1H-pyrazol-1-yl)cyclohexyl)methyl)piperidin-3-yl)amino)-2-(2,6-dioxopiperidin-3-yl)isoindoline-1,3-dione;
(E)-N-(4-((3-chloro-4-fluorophenyl)amino)-7-methoxyquinazolin-6-yl)-4-(4-((4-(2-(2,6-dioxopiperidin-3-yl)-1,3-dioxoisoindolin-5-yl)piperazin-1-yl)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-6-fluoro-1,3-dioxoisoindolin-5-yl)piperazin-1-yl)methyl)piperidin-1-yl)but-2-enamide;
(E)-N-(4-((3-chloro-4-fluorophenyl)amino)-7-methoxyquinazolin-6-yl)-4-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)but-2-enamide;
(E)-N-(4-((3-chloro-4-fluorophenyl)amino)-7-methoxyquinazolin-6-yl)-4-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)but-2-enamide;
(E)-N-(4-((3-chloro-4-fluorophenyl)amino)-7-methoxyquinazolin-6-yl)-4-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)but-2-enamide;
(E)-N-(4-((3-chloro-4-fluorophenyl)amino)-7-methoxyquinazolin-6-yl)-4-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)but-2-enamide;
(E)-N-(4-((3-chloro-4-fluorophenyl)amino)-7-methoxyquinazolin-6-yl)-4-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)but-2-enamide;
(E)-N-(4-((3-chloro-4-fluorophenyl)amino)-7-methoxyquinazolin-6-yl)-4-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)but-2-enamide;
(E)-N-(4-((3-chloro-4-fluorophenyl)amino)-7-methoxyquinazolin-6-yl)-4-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)but-2-enamide;
(E)-N-(4-((3-chloro-4-fluorophenyl)amino)-7-methoxyquinazolin-6-yl)-4-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)but-2-enamide;
(E)-N-(4-((3-chloro-4-fluorophenyl)amino)-7-methoxyquinazolin-6-yl)-4-(4-((5-(2-(2,6-dioxopiperidin-3-yl)-1,3-dioxoisoindolin-5-yl)-2,5-diazabicyclo[2.2.1]heptan-2-yl)methyl)piperidin-1-yl)but-2-enamide;
(E)-N-(4-((3-chloro-4-fluorophenyl)amino)-7-methoxyquinazolin-6-yl)-4-(4-((5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)but-2-enamide;
(E)-N-(4-((3-chloro-4-fluorophenyl)amino)-7-methoxyquinazolin-6-yl)-4-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)but-2-enamide;
(E)-N-(4-((3-chloro-4-fluorophenyl)amino)-7-methoxyquinazolin-6-yl)-4-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-1,3-dioxoisoindolin-5-yl)-3,5-dimethylpiperazin-1-yl)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-1,3-dioxoisoindolin-5-yl)-2,6-dimethylpiperazin-1-yl)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-1,3-dioxoisoindolin-5-yl)piperidin-1-yl)methyl)piperidin-1-yl)but-2-enamide;
(E)-N-(4-((3-chloro-4-fluorophenyl)amino)-7-methoxyquinazolin-6-yl)-4-(4-((4-(2-(2,6-dioxopiperidin-3-yl)-6-fluoro-1,3-dioxoisoindolin-5-yl)piperidin-1-yl)methyl)piperidin-1-yl)but-2-enamide;
(E)-N-(4-((3-chloro-4-fluorophenyl)amino)-7-methoxyquinazolin-6-yl)-4-(4-((((3R)-1-(2-(2,6-dioxopiperidin-3-yl)-1,3-dioxoisoindolin-5-yl)piperidin-3-yl)amino)methyl)piperidin-1-yl)but-2-enamide;
(E)-N-(4-((3-chloro-4-fluorophenyl)amino)-7-methoxyquinazolin-6-yl)-4-(4-((((3S)-1-(2-(2,6-dioxopiperidin-3-yl)-1,3-dioxoisoindolin-5-yl)piperidin-3-yl)amino)methyl)piperidin-1-yl)but-2-enamide;
(E)-N-(4-((3-chloro-4-fluorophenyl)amino)-7-methoxyquinazolin-6-yl)-4-(4-(((3S)-3-((2-(2,6-dioxopiperidin-3-yl)-1,3-dioxoisoindolin-5-yl)amino)piperidin-1-yl)methyl)piperidin-1-yl)but-2-enamide;
(E)-N-(4-((3-chloro-4-fluorophenyl)amino)-7-methoxyquinazolin-6-yl)-4-(4-(((3R)-3-((2-(2,6-dioxopiperidin-3-yl)-1,3-dioxoisoindolin-5-yl)amino)piperidin-1-yl)methyl)piperidin-1-yl)but-2-enamide;
N-(4-((3-chloro-4-fluorophenyl)amino)-7-(3-(4-((4-(2-(2,6-dioxopiperidin-3-yl)-1,3-dioxoisoindolin-5-yl)piperazin-1-yl)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-6-fluoro-1,3-dioxoisoindolin-5-yl)piperazin-1-yl)methyl)piperidin-1-yl)propoxy)quinazolin-6-yl)acrylamide;
N-(4-((3-chloro-4-fluorophenyl)amino)-7-(3-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)propoxy)quinazolin-6-yl)acrylamide;
N-(4-((3-chloro-4-fluorophenyl)amino)-7-(3-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)propoxy)quinazolin-6-yl)acrylamide;
N-(4-((3-chloro-4-fluorophenyl)amino)-7-(3-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)propoxy)quinazolin-6-yl)acrylamide;
N-(4-((3-chloro-4-fluorophenyl)amino)-7-(3-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)propoxy)quinazolin-6-yl)acrylamide;
N-(4-((3-chloro-4-fluorophenyl)amino)-7-(3-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)propoxy)quinazolin-6-yl)acrylamide;
N-(4-((3-chloro-4-fluorophenyl)amino)-7-(3-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)propoxy)quinazolin-6-yl)acrylamide;
N-(4-((3-chloro-4-fluorophenyl)amino)-7-(3-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)propoxy)quinazolin-6-yl)acrylamide;
N-(4-((3-chloro-4-fluorophenyl)amino)-7-(3-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)propoxy)quinazolin-6-yl)acrylamide;
N-(4-((3-chloro-4-fluorophenyl)amino)-7-(3-(4-((5-(2-(2,6-dioxopiperidin-3-yl)-1,3-dioxoisoindolin-5-yl)-2,5-diazabicyclo[2.2.1]heptan-2-yl)methyl)piperidin-1-yl)propoxy)quinazolin-6-yl)acrylamide;
N-(4-((3-chloro-4-fluorophenyl)amino)-7-(3-(4-((5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)propoxy)quinazolin-6-yl)acrylamide;
N-(4-((3-chloro-4-fluorophenyl)amino)-7-(3-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)propoxy)quinazolin-6-yl)acrylamide;
N-(4-((3-chloro-4-fluorophenyl)amino)-7-(3-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-1,3-dioxoisoindolin-5-yl)-3,5-dimethylpiperazin-1-yl)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-1,3-dioxoisoindolin-5-yl)-2,6-dimethylpiperazin-1-yl)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-1,3-dioxoisoindolin-5-yl)piperidin-1-yl)methyl)piperidin-1-yl)propoxy)quinazolin-6-yl)acrylamide;
N-(4-((3-chloro-4-fluorophenyl)amino)-7-(3-(4-((4-(2-(2,6-dioxopiperidin-3-yl)-6-fluoro-1,3-dioxoisoindolin-5-yl)piperidin-1-yl)methyl)piperidin-1-yl)propoxy)quinazolin-6-yl)acrylamide;
N-(4-((3-chloro-4-fluorophenyl)amino)-7-(3-(4-((((3R)-1-(2-(2,6-dioxopiperidin-3-yl)-1,3-dioxoisoindolin-5-yl)piperidin-3-yl)amino)methyl)piperidin-1-yl)propoxy)quinazolin-6-yl)acrylamide;
N-(4-((3-chloro-4-fluorophenyl)amino)-7-(3-(4-((((3S)-1-(2-(2,6-dioxopiperidin-3-yl)-1,3-dioxoisoindolin-5-yl)piperidin-3-yl)amino)methyl)piperidin-1-yl)propoxy)quinazolin-6-yl)acrylamide;
N-(4-((3-chloro-4-fluorophenyl)amino)-7-(3-(4-(((3S)-3-((2-(2,6-dioxopiperidin-3-yl)-1,3-dioxoisoindolin-5-yl)amino)piperidin-1-yl)methyl)piperidin-1-yl)propoxy)quinazolin-6-yl)acrylamide;
N-(4-((3-chloro-4-fluorophenyl)amino)-7-(3-(4-(((3R)-3-((2-(2,6-dioxopiperidin-3-yl)-1,3-dioxoisoindolin-5-yl)amino)piperidin-1-yl)methyl)piperidin-1-yl)propoxy)quinazolin-6-yl)acrylamide;
5-(4-((4-((4-((3,4-dichloro-2-fluorophenyl)amino)-7-methoxyquinazolin-6-yl)oxy)cyclohexyl)methyl)piperazin-1-yl)-2-(2,6-dioxopiperidin-3-yl)isoindoline-1,3-dione;
5-(4-((4-((4-((3,4-dichloro-2-fluorophenyl)amino)-7-methoxyquinazolin-6-yl)oxy)cyclohexyl)methyl)piperazin-1-yl)-2-(2,6-dioxopiperidin-3-yl)-6-fluoroisoindoline-1,3-dione;
5-(3-((4-((4-((3,4-dichloro-2-fluorophenyl)amino)-7-methoxyquinazolin-6-yl)oxy)cyclohexyl)methyl)-3,6-diazabicyclo[3.1.1]heptan-6-yl)-2-(2,6-dioxopiperidin-3-yl)isoindoline-1,3-dione;
5-(3-((4-((4-((3,4-dichloro-2-fluorophenyl)amino)-7-methoxyquinazolin-6-yl)oxy)cyclohexyl)methyl)-3,6-diazabicyclo[3.1.1]heptan-6-yl)-2-(2,6-dioxopiperidin-3-yl)-6-fluoroisoindoline-1,3-dione;
5-(6-((4-((4-((3,4-dichloro-2-fluorophenyl)amino)-7-methoxyquinazolin-6-yl)oxy)cyclohexyl)methyl)-3,6-diazabicyclo[3.1.1]heptan-3-yl)-2-(2,6-dioxopiperidin-3-yl)isoindoline-1,3-dione;
5-(6-((4-((4-((3,4-dichloro-2-fluorophenyl)amino)-7-methoxyquinazolin-6-yl)oxy)cyclohexyl)methyl)-3,6-diazabicyclo[3.1.1]heptan-3-yl)-2-(2,6-dioxopiperidin-3-yl)-6-fluoroisoindoline-1,3-dione;
5-(3-((4-((4-((3,4-dichloro-2-fluorophenyl)amino)-7-methoxyquinazolin-6-yl)oxy)cyclohexyl)methyl)-3,8-diazabicyclo[3.2.1]octan-8-yl)-2-(2,6-dioxopiperidin-3-yl)isoindoline-1,3-dione;
5-(3-((4-((4-((3,4-dichloro-2-fluorophenyl)amino)-7-methoxyquinazolin-6-yl)oxy)cyclohexyl)methyl)-3,8-diazabicyclo[3.2.1]octan-8-yl)-2-(2,6-dioxopiperidin-3-yl)-6-fluoroisoindoline-1,3-dione;
5-(8-((4-((4-((3,4-dichloro-2-fluorophenyl)amino)-7-methoxyquinazolin-6-yl)oxy)cyclohexyl)methyl)-3,8-diazabicyclo[3.2.1]octan-3-yl)-2-(2,6-dioxopiperidin-3-yl)isoindoline-1,3-dione;
5-(8-((4-((4-((3,4-dichloro-2-fluorophenyl)amino)-7-methoxyquinazolin-6-yl)oxy)cyclohexyl)methyl)-3,8-diazabicyclo[3.2.1]octan-3-yl)-2-(2,6-dioxopiperidin-3-yl)-6-fluoroisoindoline-1,3-dione;
5-(5-((4-((4-((3,4-dichloro-2-fluorophenyl)amino)-7-methoxyquinazolin-6-yl)oxy)cyclohexyl)methyl)-2,5-diazabicyclo[2.2.1]heptan-2-yl)-2-(2,6-dioxopiperidin-3-yl)isoindoline-1,3-dione;
5-(5-((4-((4-((3,4-dichloro-2-fluorophenyl)amino)-7-methoxyquinazolin-6-yl)oxy)cyclohexyl)methyl)-2,5-diazabicyclo[2.2.1]heptan-2-yl)-2-(2,6-dioxopiperidin-3-yl)-6-fluoroisoindoline-1,3-dione;
5-(5-((4-((4-((3,4-dichloro-2-fluorophenyl)amino)-7-methoxyquinazolin-6-yl)oxy)cyclohexyl)methyl)-2,5-diazabicyclo[2.2.2]octan-2-yl)-2-(2,6-dioxopiperidin-3-yl)isoindoline-1,3-dione;
5-(5-((4-((4-((3,4-dichloro-2-fluorophenyl)amino)-7-methoxyquinazolin-6-yl)oxy)cyclohexyl)methyl)-2,5-diazabicyclo[2.2.2]octan-2-yl)-2-(2,6-dioxopiperidin-3-yl)-6-fluoroisoindoline-1,3-dione;
5-(4-((4-((4-((3,4-dichloro-2-fluorophenyl)amino)-7-methoxyquinazolin-6-yl)oxy)cyclohexyl)methyl)piperazin-1-yl-2,2,3,3,5,5,6,6-d₈)-2-(2,6-dioxopiperidin-3-yl)isoindoline-1,3-dione;
5-(4-((4-((4-((3,4-dichloro-2-fluorophenyl)amino)-7-methoxyquinazolin-6-yl)oxy)cyclohexyl)methyl)piperazin-1-yl-2,2,3,3,5,5,6,6-d₈)-2-(2,6-dioxopiperidin-3-yl)-6-fluoroisoindoline-1,3-dione;
6-(4-((4-((4-((3,4-dichloro-2-fluorophenyl)amino)-7-methoxyquinazolin-6-yl)oxy)cyclohexyl)methyl)piperazin-1-yl-2,2,3,3,5,5,6,6-d₈)-2-(2,6-dioxopiperidin-3-yl)-4-fluoroisoindoline-1,3-dione;
5-(4-((4-((4-((3,4-dichloro-2-fluorophenyl)amino)-7-methoxyquinazolin-6-yl)oxy)cyclohexyl)methyl)piperazin-1-yl-2,2,3,3,5,5,6,6-d₈)-2-(2,6-dioxopiperidin-3-yl)-4-fluoroisoindoline-1,3-dione;
5-(4-((4-((4-((3,4-dichloro-2-fluorophenyl)amino)-7-methoxyquinazolin-6-yl)oxy)cyclohexyl)methyl)piperazin-1-yl-2,2,3,3,5,5,6,6-d₈)-2-(2,6-dioxopiperidin-3-yl)-4,6-difluoroisoindoline-1,3-dione;
5-(4-((4-((4-((3,4-dichloro-2-fluorophenyl)amino)-7-methoxyquinazolin-6-yl)oxy)cyclohexyl)methyl)piperazin-1-yl-2,2,3,3,5,5,6,6-d₈)-2-(2,6-dioxopiperidin-3-yl)-4,7-difluoroisoindoline-1,3-dione;
6-(4-((4-((4-((3,4-dichloro-2-fluorophenyl)amino)-7-methoxyquinazolin-6-yl)oxy)cyclohexyl)methyl)piperazin-1-yl-2,2,3,3,5,5,6,6-d₈)-2-(2,6-dioxopiperidin-3-yl)-4,5-difluoroisoindoline-1,3-dione;
5-(4-((4-((4-((3,4-dichloro-2-fluorophenyl)amino)-7-methoxyquinazolin-6-yl)oxy)cyclohexyl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((4-((4-((3,4-dichloro-2-fluorophenyl)amino)-7-methoxyquinazolin-6-yl)oxy)cyclohexyl)methyl)-2,6-dimethylpiperazin-1-yl)-2-(2,6-dioxopiperidin-3-yl)isoindoline-1,3-dione;
5-(4-((4-((4-((3,4-dichloro-2-fluorophenyl)amino)-7-methoxyquinazolin-6-yl)oxy)cyclohexyl)methyl)-2,6-dimethylpiperazin-1-yl)-2-(2,6-dioxopiperidin-3-yl)-6-fluoroisoindoline-1,3-dione;
5-(4-((4-((4-((3,4-dichloro-2-fluorophenyl)amino)-7-methoxyquinazolin-6-yl)oxy)cyclohexyl)methyl)-3,5-dimethylpiperazin-1-yl)-2-(2,6-dioxopiperidin-3-yl)isoindoline-1,3-dione;
5-(4-((4-((4-((3,4-dichloro-2-fluorophenyl)amino)-7-methoxyquinazolin-6-yl)oxy)cyclohexyl)methyl)-3,5-dimethylpiperazin-1-yl)-2-(2,6-dioxopiperidin-3-yl)-6-fluoroisoindoline-1,3-dione;
5-(1-((4-((4-((3,4-dichloro-2-fluorophenyl)amino)-7-methoxyquinazolin-6-yl)oxy)cyclohexyl)methyl)piperidin-4-yl)-2-(2,6-dioxopiperidin-3-yl)isoindoline-1,3-dione;
5-(1-((4-((4-((3,4-dichloro-2-fluorophenyl)amino)-7-methoxyquinazolin-6-yl)oxy)cyclohexyl)methyl)piperidin-4-yl)-2-(2,6-dioxopiperidin-3-yl)-6-fluoroisoindoline-1,3-dione;
5-(4-((1-(3-((4-((3-chloro-4-fluorophenyl)amino)-7-methoxyquinazolin-6-yl)oxy)propyl)piperidin-4-yl)methyl)piperazin-1-yl)-2-(2,6-dioxopiperidin-3-yl)isoindoline-1,3-dione;
5-(4-((1-(3-((4-((3-chloro-4-fluorophenyl)amino)-7-methoxyquinazolin-6-yl)oxy)propyl)piperidin-4-yl)methyl)piperazin-1-yl)-2-(2,6-dioxopiperidin-3-yl)-6-fluoroisoindoline-1,3-dione;
5-(3-((1-(3-((4-((3-chloro-4-fluorophenyl)amino)-7-methoxyquinazolin-6-yl)oxy)propyl)piperidin-4-yl)methyl)-3,6-diazabicyclo[3.1.1]heptan-6-yl)-2-(2,6-dioxopiperidin-3-yl)isoindoline-1,3-dione;
5-(3-((1-(3-((4-((3-chloro-4-fluorophenyl)amino)-7-methoxyquinazolin-6-yl)oxy)propyl)piperidin-4-yl)methyl)-3,6-diazabicyclo[3.1.1]heptan-6-yl)-2-(2,6-dioxopiperidin-3-yl)-6-fluoroisoindoline-1,3-dione;
5-(6-((1-(3-((4-((3-chloro-4-fluorophenyl)amino)-7-methoxyquinazolin-6-yl)oxy)propyl)piperidin-4-yl)methyl)-3,6-diazabicyclo[3.1.1]heptan-3-yl)-2-(2,6-dioxopiperidin-3-yl)isoindoline-1,3-dione;
5-(6-((1-(3-((4-((3-chloro-4-fluorophenyl)amino)-7-methoxyquinazolin-6-yl)oxy)propyl)piperidin-4-yl)methyl)-3,6-diazabicyclo[3.1.1]heptan-3-yl)-2-(2,6-dioxopiperidin-3-yl)-6-fluoroisoindoline-1,3-dione;
5-(3-((1-(3-((4-((3-chloro-4-fluorophenyl)amino)-7-methoxyquinazolin-6-yl)oxy)propyl)piperidin-4-yl)methyl)-3,8-diazabicyclo[3.2.1]octan-8-yl)-2-(2,6-dioxopiperidin-3-5-(3-((1-(3-((4-((3-chloro-4-fluorophenyl)amino)-7-methoxyquinazolin-6-yl)oxy)propyl)piperidin-4-yl)methyl)-3,8-diazabicyclo[3.2.1]octan-8-yl)-2-(2,6-dioxopiperidin-3-yl)-6-fluoroisoindoline-1,3-dione;
5-(8-((1-(3-((4-((3-chloro-4-fluorophenyl)amino)-7-methoxyquinazolin-6-yl)oxy)propyl)piperidin-4-yl)methyl)-3,8-diazabicyclo[3.2.1]octan-3-yl)-2-(2,6-dioxopiperidin-3-yl)isoindoline-1,3-dione;
5-(8-((1-(3-((4-((3-chloro-4-fluorophenyl)amino)-7-methoxyquinazolin-6-yl)oxy)propyl)piperidin-4-yl)methyl)-3,8-diazabicyclo[3.2.1]octan-3-yl)-2-(2,6-dioxopiperidin-3-yl)-6-fluoroisoindoline-1,3-dione;
5-(5-((1-(3-((4-((3-chloro-4-fluorophenyl)amino)-7-methoxyquinazolin-6-yl)oxy)propyl)piperidin-4-yl)methyl)-2,5-diazabicyclo[2.2.1]heptan-2-yl)-2-(2,6-dioxopiperidin-3-yl)isoindoline-1,3-dione;
5-(5-((1-(3-((4-((3-chloro-4-fluorophenyl)amino)-7-methoxyquinazolin-6-yl)oxy)propyl)piperidin-4-yl)methyl)-2,5-diazabicyclo[2.2.1]heptan-2-yl)-2-(2,6-dioxopiperidin-3-yl)-6-fluoroisoindoline-1,3-dione;
5-(5-((1-(3-((4-((3-chloro-4-fluorophenyl)amino)-7-methoxyquinazolin-6-yl)oxy)propyl)piperidin-4-yl)methyl)-2,5-diazabicyclo[2.2.2]octan-2-yl)-2-(2,6-dioxopiperidin-3-yl)isoindoline-1,3-dione;
5-(5-((1-(3-((4-((3-chloro-4-fluorophenyl)amino)-7-methoxyquinazolin-6-yl)oxy)propyl)piperidin-4-yl)methyl)-2,5-diazabicyclo[2.2.2]octan-2-yl)-2-(2,6-dioxopiperidin-3-yl)-6-fluoroisoindoline-1,3-dione;
5-(4-((1-(3-((4-((3-chloro-4-fluorophenyl)amino)-7-methoxyquinazolin-6-yl)oxy)propyl)piperidin-4-yl)methyl)piperazin-1-yl-2,2,3,3,5,5,6,6-d₈)-2-(2,6-dioxopiperidin-3-yl)isoindoline-1,3-dione;
5-(4-((1-(3-((4-((3-chloro-4-fluorophenyl)amino)-7-methoxyquinazolin-6-yl)oxy)propyl)piperidin-4-yl)methyl)piperazin-1-yl-2,2,3,3,5,5,6,6-d₈)-2-(2,6-dioxopiperidin-3-yl)-6-fluoroisoindoline-1,3-dione;
6-(4-((1-(3-((4-((3-chloro-4-fluorophenyl)amino)-7-methoxyquinazolin-6-yl)oxy)propyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(3-((4-((3-chloro-4-fluorophenyl)amino)-7-methoxyquinazolin-6-yl)oxy)propyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(3-((4-((3-chloro-4-fluorophenyl)amino)-7-methoxyquinazolin-6-yl)oxy)propyl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
5-(4-((1-(3-((4-((3-chloro-4-fluorophenyl)amino)-7-methoxyquinazolin-6-yl)oxy)propyl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
6-(4-((1-(3-((4-((3-chloro-4-fluorophenyl)amino)-7-methoxyquinazolin-6-yl)oxy)propyl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
5-(4-((1-(3-((4-((3-chloro-4-fluorophenyl)amino)-7-methoxyquinazolin-6-yl)oxy)propyl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((1-(3-((4-((3-chloro-4-fluorophenyl)amino)-7-methoxyquinazolin-6-yl)oxy)propyl)piperidin-4-yl)methyl)-2,6-dimethylpiperazin-1-yl)-2-(2,6-dioxopiperidin-3-yl)isoindoline-1,3-dione;
5-(4-((1-(3-((4-((3-chloro-4-fluorophenyl)amino)-7-methoxyquinazolin-6-yl)oxy)propyl)piperidin-4-yl)methyl)-2,6-dimethylpiperazin-1-yl)-2-(2,6-dioxopiperidin-3-yl)-6-fluoroisoindoline-1,3-dione;
5-(4-((1-(3-((4-((3-chloro-4-fluorophenyl)amino)-7-methoxyquinazolin-6-yl)oxy)propyl)piperidin-4-yl)methyl)-3,5-dimethylpiperazin-1-yl)-2-(2,6-dioxopiperidin-3-yl)isoindoline-1,3-dione;
5-(4-((1-(3-((4-((3-chloro-4-fluorophenyl)amino)-7-methoxyquinazolin-6-yl)oxy)propyl)piperidin-4-yl)methyl)-3,5-dimethylpiperazin-1-yl)-2-(2,6-dioxopiperidin-3-yl)-6-fluoroisoindoline-1,3-dione;
5-(1-((1-(3-((4-((3-chloro-4-fluorophenyl)amino)-7-methoxyquinazolin-6-yl)oxy)propyl)piperidin-4-yl)methyl)piperidin-4-yl)-2-(2,6-dioxopiperidin-3-yl)isoindoline-1,3-dione;
5-(1-((1-(3-((4-((3-chloro-4-fluorophenyl)amino)-7-methoxyquinazolin-6-yl)oxy)propyl)piperidin-4-yl)methyl)piperidin-4-yl)-2-(2,6-dioxopiperidin-3-yl)-6-fluoroisoindoline-1,3-dione;
5-((R)-3-(((1-(3-((4-((3-chloro-4-fluorophenyl)amino)-7-methoxyquinazolin-6-yl)oxy)propyl)piperidin-4-yl)methyl)amino)piperidin-1-yl)-2-(2,6-dioxopiperidin-3-yl)isoindoline-1,3-dione;
5-((S)-3-(((1-(3-((4-((3-chloro-4-fluorophenyl)amino)-7-methoxyquinazolin-6-yl)oxy)propyl)piperidin-4-yl)methyl)amino)piperidin-1-yl)-2-(2,6-dioxopiperidin-3-yl)isoindoline-1,3-dione;
5-(((S)-1-((1-(3-((4-((3-chloro-4-fluorophenyl)amino)-7-methoxyquinazolin-6-yl)oxy)propyl)piperidin-4-yl)methyl)piperidin-3-yl)amino)-2-(2,6-dioxopiperidin-3-yl)isoindoline-1,3-dione;
5-(((R)-1-((1-(3-((4-((3-chloro-4-fluorophenyl)amino)-7-methoxyquinazolin-6-yl)oxy)propyl)piperidin-4-yl)methyl)piperidin-3-yl)amino)-2-(2,6-dioxopiperidin-3-yl)isoindoline-1,3-dione;
N-(2-((2-(4-((4-(2-(2,6-dioxopiperidin-3-yl)-1,3-dioxoisoindolin-5-yl)piperazin-1-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-6-fluoro-1,3-dioxoisoindolin-5-yl)piperazin-1-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((5-(2-(2,6-dioxopiperidin-3-yl)-1,3-dioxoisoindolin-5-yl)-2,5-diazabicyclo[2.2.1]heptan-2-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-1,3-dioxoisoindolin-5-yl)-3,5-dimethylpiperazin-1-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-1,3-dioxoisoindolin-5-yl)-2,6-dimethylpiperazin-1-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-1,3-dioxoisoindolin-5-yl)piperidin-1-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((4-(2-(2,6-dioxopiperidin-3-yl)-6-fluoro-1,3-dioxoisoindolin-5-yl)piperidin-1-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((((3R)-1-(2-(2,6-dioxopiperidin-3-yl)-1,3-dioxoisoindolin-5-yl)piperidin-3-yl)amino)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-((((3S)-1-(2-(2,6-dioxopiperidin-3-yl)-1,3-dioxoisoindolin-5-yl)piperidin-3-yl)amino)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-(((3S)-3-((2-(2,6-dioxopiperidin-3-yl)-1,3-dioxoisoindolin-5-yl)amino)piperidin-1-yl)methyl)piperidin-1-yl)ethyl)(methyl)amino)-4-methoxy-5-((4-(1-methyl-1H-indol-3-yl)pyrimidin-2-yl)amino)phenyl)acrylamide;
N-(2-((2-(4-(((3R)-3-((2-(2,6-dioxopiperidin-3-yl)-1,3-dioxoisoindolin-5-yl)amino)piperidin-1-yl)methyl)piperidin-1-yl)ethyl)(methyl)amino)-4-methoxy-5-((4-(1-methyl-1H-indol-3-yl)pyrimidin-2-yl)amino)phenyl)acrylamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-1,3-dioxoisoindolin-5-yl)piperazin-1-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-6-fluoro-1,3-dioxoisoindolin-5-yl)piperazin-1-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((5-(2-(2,6-dioxopiperidin-3-yl)-1,3-dioxoisoindolin-5-yl)-2,5-diazabicyclo[2.2.1]heptan-2-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-1,3-dioxoisoindolin-5-yl)-3,5-dimethylpiperazin-1-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-1,3-dioxoisoindolin-5-yl)-2,6-dimethylpiperazin-1-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-1,3-dioxoisoindolin-5-yl)piperidin-1-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-6-fluoro-1,3-dioxoisoindolin-5-yl)piperidin-1-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((((3R)-1-(2-(2,6-dioxopiperidin-3-yl)-1,3-dioxoisoindolin-5-yl)piperidin-3-yl)amino)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-((((3S)-1-(2-(2,6-dioxopiperidin-3-yl)-1,3-dioxoisoindolin-5-yl)piperidin-3-yl)amino)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-(((3S)-3-((2-(2,6-dioxopiperidin-3-yl)-1,3-dioxoisoindolin-5-yl)amino)piperidin-1-yl)methyl)piperidin-1-yl)but-2-enamide;
(E)-N-(4-((3-chloro-4-fluorophenyl)amino)-7-(((S)-tetrahydrofuran-3-yl)oxy)quinazolin-6-yl)-4-(4-(((3R)-3-((2-(2,6-dioxopiperidin-3-yl)-1,3-dioxoisoindolin-5-yl)amino)piperidin-1-yl)methyl)piperidin-1-yl)but-2-enamide;
2-(6-(6-(4-((4-(2-(2,6-dioxopiperidin-3-yl)-1,3-dioxoisoindolin-5-yl)piperazin-1-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-6-fluoro-1,3-dioxoisoindolin-5-yl)piperazin-1-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((5-(2-(2,6-dioxopiperidin-3-yl)-1,3-dioxoisoindolin-5-yl)-2,5-diazabicyclo[2.2.1]heptan-2-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-1,3-dioxoisoindolin-5-yl)-3,5-dimethylpiperazin-1-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-1,3-dioxoisoindolin-5-yl)-2,6-dimethylpiperazin-1-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-1,3-dioxoisoindolin-5-yl)piperidin-1-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((4-(2-(2,6-dioxopiperidin-3-yl)-6-fluoro-1,3-dioxoisoindolin-5-yl)piperidin-1-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((((3R)-1-(2-(2,6-dioxopiperidin-3-yl)-1,3-dioxoisoindolin-5-yl)piperidin-3-yl)amino)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-((((3S)-1-(2-(2,6-dioxopiperidin-3-yl)-1,3-dioxoisoindolin-5-yl)piperidin-3-yl)amino)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-(((3S)-3-((2-(2,6-dioxopiperidin-3-yl)-1,3-dioxoisoindolin-5-yl)amino)piperidin-1-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(6-(6-(4-(((3R)-3-((2-(2,6-dioxopiperidin-3-yl)-1,3-dioxoisoindolin-5-yl)amino)piperidin-1-yl)methyl)piperidin-1-yl)pyridazin-3-yl)-1-oxoisoindolin-2-yl)-2-(5-fluoro-2-hydroxyphenyl)-N-(thiazol-2-yl)acetamide;
2-(2,6-dioxopiperidin-3-yl)-5-(4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl)isoindoline-1,3-dione;
2-(2,6-dioxopiperidin-3-yl)-5-fluoro-6-(4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl)isoindoline-1,3-dione;
2-(2,6-dioxopiperidin-3-yl)-5-(3-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)-3,6-diazabicyclo[3.1.1]heptan-6-yl)isoindoline-1,3-dione;
2-(2,6-dioxopiperidin-3-yl)-5-fluoro-6-(3-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)-3,6-diazabicyclo[3.1.1]heptan-6-yl)isoindoline-1,3-dione;
2-(2,6-dioxopiperidin-3-yl)-5-(6-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)-3,6-diazabicyclo[3.1.1]heptan-3-yl)isoindoline-1,3-dione;
2-(2,6-dioxopiperidin-3-yl)-5-fluoro-6-(6-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)-3,6-diazabicyclo[3.1.1]heptan-3-yl)isoindoline-1,3-dione;
2-(2,6-dioxopiperidin-3-yl)-5-(3-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)-3,8-diazabicyclo[3.2.1]octan-8-yl)isoindoline-1,3-dione;
2-(2,6-dioxopiperidin-3-yl)-5-fluoro-6-(3-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)-3,8-diazabicyclo[3.2.1]octan-8-yl)isoindoline-1,3-dione;
2-(2,6-dioxopiperidin-3-yl)-5-(8-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)-3,8-diazabicyclo[3.2.1]octan-3-yl)isoindoline-1,3-dione;
2-(2,6-dioxopiperidin-3-yl)-5-fluoro-6-(8-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)-3,8-diazabicyclo[3.2.1]octan-3-yl)isoindoline-1,3-dione;
2-(2,6-dioxopiperidin-3-yl)-5-fluoro-6-(5-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)-2,5-diazabicyclo[2.2.1]heptan-2-yl)isoindoline-1,3-dione;
2-(2,6-dioxopiperidin-3-yl)-5-(5-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)-2,5-diazabicyclo[2.2.1]heptan-2-yl)isoindoline-1,3-dione;
2-(2,6-dioxopiperidin-3-yl)-5-(5-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)-2,5-diazabicyclo[2.2.2]octan-2-yl)isoindoline-1,3-dione;
2-(2,6-dioxopiperidin-3-yl)-5-fluoro-6-(5-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)-2,5-diazabicyclo[2.2.2]octan-2-yl)isoindoline-1,3-dione;
2-(2,6-dioxopiperidin-3-yl)-5-(4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl-2,2,3,3,5,5,6,6-d₈)isoindoline-1,3-dione;
2-(2,6-dioxopiperidin-3-yl)-5-fluoro-6-(4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl-2,2,3,3,5,5,6,6-d₈)isoindoline-1,3-dione;
2-(2,6-dioxopiperidin-3-yl)-4-fluoro-6-(4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl-2,2,3,3,5,5,6,6-d₈)isoindoline-1,3-dione;
2-(2,6-dioxopiperidin-3-yl)-4-fluoro-5-(4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl-2,2,3,3,5,5,6,6-d₈)isoindoline-1,3-dione;
2-(2,6-dioxopiperidin-3-yl)-4,6-difluoro-5-(4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl-2,2,3,3,5,5,6,6-d₈)isoindoline-1,3-dione;
2-(2,6-dioxopiperidin-3-yl)-4,7-difluoro-5-(4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl-2,2,3,3,5,5,6,6-d₈)isoindoline-1,3-dione;
2-(2,6-dioxopiperidin-3-yl)-4,5-difluoro-6-(4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl-2,2,3,3,5,5,6,6-d₈)isoindoline-1,3-dione;
2-(2,6-dioxopiperidin-3-yl)-4,5,7-trifluoro-6-(4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl-2,2,3,3,5,5,6,6-d₈)isoindoline-1,3-dione;
5-(2,6-dimethyl-4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl)-2-(2,6-dioxopiperidin-3-yl)isoindoline-1,3-dione;
5-(2,6-dimethyl-4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl)-2-(2,6-dioxopiperidin-3-yl)-6-fluoroisoindoline-1,3-dione;
5-(3,5-dimethyl-4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl)-2-(2,6-dioxopiperidin-3-yl)isoindoline-1,3-dione;
5-(3,5-dimethyl-4-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperazin-1-yl)-2-(2,6-dioxopiperidin-3-yl)-6-fluoroisoindoline-1,3-dione;
2-(2,6-dioxopiperidin-3-yl)-5-(1-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperidin-4-yl)isoindoline-1,3-dione;
2-(2,6-dioxopiperidin-3-yl)-5-fluoro-6-(1-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperidin-4-yl)isoindoline-1,3-dione;
2-(2,6-dioxopiperidin-3-yl)-5-((R)-3-(((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)amino)piperidin-1-yl)isoindoline-1,3-dione;
2-(2,6-dioxopiperidin-3-yl)-5-((S)-3-(((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)amino)piperidin-1-yl)isoindoline-1,3-dione;
2-(2,6-dioxopiperidin-3-yl)-5-(((S)-1-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperidin-3-yl)amino)isoindoline-1,3-dione;
2-(2,6-dioxopiperidin-3-yl)-5-(((R)-1-((1-(((R,E)-1¹,2⁶,7-trimethyl-3-oxo-5²,5³-dihydro-1¹H,5¹H-11-oxa-4-aza-5(2,1)-benzo[d]imidazola-2(2,4)-pyridina-1(4,5)-pyrazolacycloundecaphane-5⁶-yl)methyl)piperidin-4-yl)methyl)piperidin-3-yl)amino)isoindoline-1,3-dione;
5-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperazin-1-yl)-2-(2,6-dioxopiperidin-3-yl)isoindoline-1,3-dione;
5-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperazin-1-yl)-2-(2,6-dioxopiperidin-3-yl)-6-fluoroisoindoline-1,3-dione;
5-(3-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)-3,6-diazabicyclo[3.1.1]heptan-6-yl)-2-(2,6-dioxopiperidin-3-yl)isoindoline-1,3-dione;
5-(3-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)-3,6-diazabicyclo[3.1.1]heptan-6-yl)-2-(2,6-dioxopiperidin-3-yl)-6-fluoroisoindoline-1,3-dione;
5-(6-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)-3,6-diazabicyclo[3.1.1]heptan-3-yl)-2-(2,6-dioxopiperidin-3-yl)isoindoline-1,3-dione;
5-(6-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)-3,6-diazabicyclo[3.1.1]heptan-3-yl)-2-(2,6-dioxopiperidin-3-yl)-6-fluoroisoindoline-1,3-dione;
5-(5-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)-2,5-diazabicyclo[2.2.1]heptan-2-yl)-2-(2,6-dioxopiperidin-3-yl)isoindoline-1,3-dione;
5-(5-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)-2,5-diazabicyclo[2.2.1]heptan-2-yl)-2-(2,6-dioxopiperidin-3-yl)-6-fluoroisoindoline-1,3-dione;
5-(5-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)-2,5-diazabicyclo[2.2.2]octan-2-yl)-2-(2,6-dioxopiperidin-3-yl)isoindoline-1,3-dione;
5-(5-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)-2,5-diazabicyclo[2.2.2]octan-2-yl)-2-(2,6-dioxopiperidin-3-yl)-6-fluoroisoindoline-1,3-dione;
5-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperazin-1-yl-2,2,3,3,5,5,6,6-d₈)-2-(2,6-dioxopiperidin-3-yl)isoindoline-1,3-dione;
5-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperazin-1-yl-2,2,3,3,5,5,6,6-d₈)-2-(2,6-dioxopiperidin-3-yl)-6-fluoroisoindoline-1,3-dione;
6-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
5-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
6-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
5-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)-2,6-dimethylpiperazin-1-yl)-2-(2,6-dioxopiperidin-3-yl)isoindoline-1,3-dione;
5-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)-2,6-dimethylpiperazin-1-yl)-2-(2,6-dioxopiperidin-3-yl)-6-fluoroisoindoline-1,3-dione;
5-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)-3,5-dimethylpiperazin-1-yl)-2-(2,6-dioxopiperidin-3-yl)isoindoline-1,3-dione;
5-(4-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)-3,5-dimethylpiperazin-1-yl)-2-(2,6-dioxopiperidin-3-yl)-6-fluoroisoindoline-1,3-dione;
5-(1-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperidin-4-yl)-2-(2,6-dioxopiperidin-3-yl)isoindoline-1,3-dione;
5-(1-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperidin-4-yl)-2-(2,6-dioxopiperidin-3-yl)-6-fluoroisoindoline-1,3-dione;
5-((R)-3-(((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)amino)piperidin-1-yl)-2-(2,6-dioxopiperidin-3-yl)isoindoline-1,3-dione;
5-((S)-3-(((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)amino)piperidin-1-yl)-2-(2,6-dioxopiperidin-3-yl)isoindoline-1,3-dione;
5-(((S)-1-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperidin-3-yl)amino)-2-(2,6-dioxopiperidin-3-yl)isoindoline-1,3-dione;
5-(((R)-1-((1-(6-((2-(1-(cyclopropylsulfonyl)-1H-pyrazol-4-yl)pyrimidin-4-yl)amino)-1-isopropyl-1H-pyrazolo[4,3-c]pyridin-3-yl)piperidin-4-yl)methyl)piperidin-3-yl)amino)-2-(2,6-dioxopiperidin-3-yl)isoindoline-1,3-dione;
2-(2,6-dioxopiperidin-3-yl)-5-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)piperazin-1-yl)isoindoline-1,3-dione;
2-(2,6-dioxopiperidin-3-yl)-5-fluoro-6-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)piperazin-1-yl)isoindoline-1,3-dione;
2-(2,6-dioxopiperidin-3-yl)-5-(3-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-3,6-diazabicyclo[3.1.1]heptan-6-yl)isoindoline-1,3-dione;
2-(2,6-dioxopiperidin-3-yl)-5-fluoro-6-(3-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-3,6-diazabicyclo[3.1.1]heptan-6-yl)isoindoline-1,3-dione;
2-(2,6-dioxopiperidin-3-yl)-5-(6-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-3,6-diazabicyclo[3.1.1]heptan-3-yl)isoindoline-1,3-dione;
2-(2,6-dioxopiperidin-3-yl)-5-fluoro-6-(6-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-3,6-diazabicyclo[3.1.1]heptan-3-yl)isoindoline-1,3-dione;
2-(2,6-dioxopiperidin-3-yl)-5-(3-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-3,8-diazabicyclo[3.2.1]octan-8-yl)isoindoline-1,3-dione;
2-(2,6-dioxopiperidin-3-yl)-5-fluoro-6-(3-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-3,8-diazabicyclo[3.2.1]octan-8-yl)isoindoline-1,3-dione;
2-(2,6-dioxopiperidin-3-yl)-5-(8-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-3,8-diazabicyclo[3.2.1]octan-3-2-(2,6-dioxopiperidin-3-yl)-5-fluoro-6-(8-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-3,8-diazabicyclo[3.2.1]octan-3-yl)isoindoline-1,3-dione;
2-(2,6-dioxopiperidin-3-yl)-5-(5-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-2,5-diazabicyclo[2.2.1]heptan-2-yl)isoindoline-1,3-dione;
2-(2,6-dioxopiperidin-3-yl)-5-fluoro-6-(5-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-2,5-diazabicyclo[2.2.1]heptan-2-yl)isoindoline-1,3-dione;
2-(2,6-dioxopiperidin-3-yl)-5-(5-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-2,5-diazabicyclo[2.2.2]octan-2-yl)isoindoline-1,3-dione;
2-(2,6-dioxopiperidin-3-yl)-5-fluoro-6-(5-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-2,5-diazabicyclo[2.2.2]octan-2-yl)isoindoline-1,3-dione;
2-(2,6-dioxopiperidin-3-yl)-5-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)piperazin-1-yl-2,2,3,3,5,5,6,6-d₈)isoindoline-1,3-dione;
2-(2,6-dioxopiperidin-3-yl)-5-fluoro-6-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)piperazin-1-yl-2,2,3,3,5,5,6,6-d₈)isoindoline-1,3-dione;
2-(2,6-dioxopiperidin-3-yl)-4-fluoro-6-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)piperazin-1-yl-2,2,3,3,5,5,6,6-d₈)isoindoline-1,3-dione;
2-(2,6-dioxopiperidin-3-yl)-4-fluoro-5-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)piperazin-1-yl-2,2,3,3,5,5,6,6-d₈)isoindoline-1,3-dione;
2-(2,6-dioxopiperidin-3-yl)-4,6-difluoro-5-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)piperazin-1-yl-2,2,3,3,5,5,6,6-d₈)isoindoline-1,3-dione;
2-(2,6-dioxopiperidin-3-yl)-4,7-difluoro-5-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)piperazin-1-yl-2,2,3,3,5,5,6,6-d₈)isoindoline-1,3-dione;
2-(2,6-dioxopiperidin-3-yl)-4,5-difluoro-6-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)piperazin-1-yl-2,2,3,3,5,5,6,6-d₈)isoindoline-1,3-dione;
2-(2,6-dioxopiperidin-3-yl)-4,5,7-trifluoro-6-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)piperazin-1-yl-2,2,3,3,5,5,6,6-d₈)isoindoline-1,3-dione;
2-(2,6-dioxopiperidin-3-yl)-5-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-2,6-dimethylpiperazin-1-yl)isoindoline-1,3-dione;
2-(2,6-dioxopiperidin-3-yl)-5-fluoro-6-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-2,6-dimethylpiperazin-1-yl)isoindoline-1,3-dione;
2-(2,6-dioxopiperidin-3-yl)-5-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-3,5-dimethylpiperazin-1-yl)isoindoline-1,3-dione;
2-(2,6-dioxopiperidin-3-yl)-5-fluoro-6-(4-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)-3,5-dimethylpiperazin-1-yl)isoindoline-1,3-dione;
2-(2,6-dioxopiperidin-3-yl)-5-(1-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)piperidin-4-yl)isoindoline-1,3-dione;
2-(2,6-dioxopiperidin-3-yl)-5-fluoro-6-(1-((1-(1-isopropyl-6-((2-(4-methoxypiperidin-1-yl)pyrimidin-4-yl)amino)-1H-pyrazolo[4,3-c]pyridin-3-yl)piperidin-4-yl)methyl)piperidin-4-yl)isoindoline-1,3-dione;
N-(tert-butyl)-3-((2-((4-(2-(4-((4-(2-(2,6-dioxopiperidin-3-yl)-1,3-dioxoisoindolin-5-yl)piperazin-1-yl)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-6-fluoro-1,3-dioxoisoindolin-5-yl)piperazin-1-yl)methyl)piperidin-1-yl)ethoxy)phenyl)amino)-5-methylpyrimidin-4-yl)amino)benzenesulfonamide;
N-(tert-butyl)-3-((2-((4-(2-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)ethoxy)phenyl)amino)-5-methylpyrimidin-4-yl)amino)benzenesulfonamide;
N-(tert-butyl)-3-((2-((4-(2-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)ethoxy)phenyl)amino)-5-methylpyrimidin-4-yl)amino)benzenesulfonamide;
N-(tert-butyl)-3-((2-((4-(2-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)ethoxy)phenyl)amino)-5-methylpyrimidin-4-yl)amino)benzenesulfonamide;
N-(tert-butyl)-3-((2-((4-(2-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)ethoxy)phenyl)amino)-5-methylpyrimidin-4-yl)amino)benzenesulfonamide;
N-(tert-butyl)-3-((2-((4-(2-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)ethoxy)phenyl)amino)-5-methylpyrimidin-4-yl)amino)benzenesulfonamide;
N-(tert-butyl)-3-((2-((4-(2-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)ethoxy)phenyl)amino)-5-methylpyrimidin-4-yl)amino)benzenesulfonamide;
N-(tert-butyl)-3-((2-((4-(2-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)ethoxy)phenyl)amino)-5-methylpyrimidin-4-yl)amino)benzenesulfonamide;
N-(tert-butyl)-3-((2-((4-(2-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)ethoxy)phenyl)amino)-5-methylpyrimidin-4-yl)amino)benzenesulfonamide;
N-(tert-butyl)-3-((2-((4-(2-(4-((5-(2-(2,6-dioxopiperidin-3-yl)-1,3-dioxoisoindolin-5-yl)-2,5-diazabicyclo[2.2.1]heptan-2-yl)methyl)piperidin-1-yl)ethoxy)phenyl)amino)-5-methylpyrimidin-4-yl)amino)benzenesulfonamide;
N-(tert-butyl)-3-((2-((4-(2-(4-((5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)ethoxy)phenyl)amino)-5-methylpyrimidin-4-yl)amino)benzenesulfonamide;
N-(tert-butyl)-3-((2-((4-(2-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)ethoxy)phenyl)amino)-5-methylpyrimidin-4-yl)amino)benzenesulfonamide;
N-(tert-butyl)-3-((2-((4-(2-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-1,3-dioxoisoindolin-5-yl)-3,5-dimethylpiperazin-1-yl)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-1,3-dioxoisoindolin-5-yl)-2,6-dimethylpiperazin-1-yl)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-1,3-dioxoisoindolin-5-yl)piperidin-1-yl)methyl)piperidin-1-yl)ethoxy)phenyl)amino)-5-methylpyrimidin-4-yl)amino)benzenesulfonamide;
N-(tert-butyl)-3-((2-((4-(2-(4-((4-(2-(2,6-dioxopiperidin-3-yl)-6-fluoro-1,3-dioxoisoindolin-5-yl)piperidin-1-yl)methyl)piperidin-1-yl)ethoxy)phenyl)amino)-5-methylpyrimidin-4-yl)amino)benzenesulfonamide;
N-(tert-butyl)-3-((2-((4-(2-(4-((((3R)-1-(2-(2,6-dioxopiperidin-3-yl)-1,3-dioxoisoindolin-5-yl)piperidin-3-yl)amino)methyl)piperidin-1-yl)ethoxy)phenyl)amino)-5-methylpyrimidin-4-yl)amino)benzenesulfonamide;
N-(tert-butyl)-3-((2-((4-(2-(4-((((3S)-1-(2-(2,6-dioxopiperidin-3-yl)-1,3-dioxoisoindolin-5-yl)piperidin-3-yl)amino)methyl)piperidin-1-yl)ethoxy)phenyl)amino)-5-methylpyrimidin-4-yl)amino)benzenesulfonamide;
N-(tert-butyl)-3-((2-((4-(2-(4-(((3S)-3-((2-(2,6-dioxopiperidin-3-yl)-1,3-dioxoisoindolin-5-yl)amino)piperidin-1-yl)methyl)piperidin-1-yl)ethoxy)phenyl)amino)-5-methylpyrimidin-4-yl)amino)benzenesulfonamide;
N-(tert-butyl)-3-((2-((4-(2-(4-(((3R)-3-((2-(2,6-dioxopiperidin-3-yl)-1,3-dioxoisoindolin-5-yl)amino)piperidin-1-yl)methyl)piperidin-1-yl)ethoxy)phenyl)amino)-5-methylpyrimidin-4-yl)amino)benzenesulfonamide;
4-(4-((4-(2-(2,6-dioxopiperidin-3-yl)-1,3-dioxoisoindolin-5-yl)piperazin-1-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-6-fluoro-1,3-dioxoisoindolin-5-yl)piperazin-1-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((5-(2-(2,6-dioxopiperidin-3-yl)-1,3-dioxoisoindolin-5-yl)-2,5-diazabicyclo[2.2.1]heptan-2-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-1,3-dioxoisoindolin-5-yl)-3,5-dimethylpiperazin-1-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-1,3-dioxoisoindolin-5-yl)-2,6-dimethylpiperazin-1-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-1,3-dioxoisoindolin-5-yl)piperidin-1-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((4-(2-(2,6-dioxopiperidin-3-yl)-6-fluoro-1,3-dioxoisoindolin-5-yl)piperidin-1-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((((3R)-1-(2-(2,6-dioxopiperidin-3-yl)-1,3-dioxoisoindolin-5-yl)piperidin-3-yl)amino)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-((((3S)-1-(2-(2,6-dioxopiperidin-3-yl)-1,3-dioxoisoindolin-5-yl)piperidin-3-yl)amino)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-(((3S)-3-((2-(2,6-dioxopiperidin-3-yl)-1,3-dioxoisoindolin-5-yl)amino)piperidin-1-yl)methyl)piperidin-1-yl)-N-(5-((R)-2-methoxy-2-phenylacetyl)-1,4,5,6-tetrahydropyrrolo[3,4-c]pyrazol-3-yl)benzamide;
4-(4-(((3R)-3-((2-(2,6-dioxopiperidin-3-yl)-1,3-dioxoisoindolin-5-yl)amino)piperidin-1-yl)methyl)piperidin-1-yl)-N-(5-((R)-2-methoxy-2-phenylacetyl)-1,4,5,6-tetrahydropyrrolo[3,4-c]pyrazol-3-yl)benzamide;
5-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperazin-1-yl)-2-(2,6-dioxopiperidin-3-yl)isoindoline-1,3-dione;
5-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperazin-1-yl)-2-(2,6-dioxopiperidin-3-yl)-6-fluoroisoindoline-1,3-dione;
5-(3-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-3,6-diazabicyclo[3.1.1]heptan-6-yl)-2-(2,6-dioxopiperidin-3-yl)isoindoline-1,3-dione;
5-(3-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-3,6-diazabicyclo[3.1.1]heptan-6-yl)-2-(2,6-dioxopiperidin-3-yl)-6-fluoroisoindoline-1,3-dione;
5-(6-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-3,6-diazabicyclo[3.1.1]heptan-3-yl)-2-(2,6-dioxopiperidin-3-yl)isoindoline-1,3-dione;
5-(6-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-3,6-diazabicyclo[3.1.1]heptan-3-yl)-2-(2,6-dioxopiperidin-3-yl)-6-fluoroisoindoline-1,3-dione;
5-(3-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-3,8-diazabicyclo[3.2.1]octan-8-yl)-2-(2,6-dioxopiperidin-3-yl)isoindoline-1,3-dione;
5-(3-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-3,8-diazabicyclo[3.2.1]octan-8-yl)-2-(2,6-dioxopiperidin-3-yl)-6-fluoroisoindoline-1,3-dione;
5-(3-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-3,8-diazabicyclo[3.2.1]octan-8-yl)-2-(2,6-dioxopiperidin-3-yl)-6-fluoroisoindoline-1,3-dione;
6-(3-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-3,8-diazabicyclo[3.2.1]octan-8-yl)-2-(2,6-dioxopiperidin-3-yl)-4-fluoroisoindoline-1,3-dione;
5-(8-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-3,8-diazabicyclo[3.2.1]octan-3-yl)-2-(2,6-dioxopiperidin-3-yl)isoindoline-1,3-dione;
5-(8-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-3,8-diazabicyclo[3.2.1]octan-3-yl)-2-(2,6-dioxopiperidin-3-yl)-6-fluoroisoindoline-1,3-dione;
5-(5-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-2,5-diazabicyclo[2.2.1]heptan-2-yl)-2-(2,6-dioxopiperidin-3-yl)isoindoline-1,3-dione;
5-(5-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-2,5-diazabicyclo[2.2.1]heptan-2-yl)-2-(2,6-dioxopiperidin-3-yl)-6-fluoroisoindoline-1,3-dione;
5-(5-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-2,5-diazabicyclo[2.2.2]octan-2-yl)-2-(2,6-dioxopiperidin-3-yl)isoindoline-1,3-dione;
5-(5-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-2,5-diazabicyclo[2.2.2]octan-2-yl)-2-(2,6-dioxopiperidin-3-yl)-6-fluoroisoindoline-1,3-dione;
5-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperazin-1-yl-2,2,3,3,5,5,6,6-d₈)-2-(2,6-dioxopiperidin-3-yl)isoindoline-1,3-dione;
5-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperazin-1-yl-2,2,3,3,5,5,6,6-d₈)-2-(2,6-dioxopiperidin-3-yl)-6-fluoroisoindoline-1,3-dione;
6-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
5-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
6-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
5-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-2,6-dimethylpiperazin-1-yl)-2-(2,6-dioxopiperidin-3-yl)isoindoline-1,3-dione;
5-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-2,6-dimethylpiperazin-1-yl)-2-(2,6-dioxopiperidin-3-yl)-6-fluoroisoindoline-1,3-dione;
5-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-3,5-dimethylpiperazin-1-yl)-2-(2,6-dioxopiperidin-3-yl)isoindoline-1,3-dione;
5-(4-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)-3,5-dimethylpiperazin-1-yl)-2-(2,6-dioxopiperidin-3-yl)-6-fluoroisoindoline-1,3-dione;
5-(1-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperidin-4-yl)-2-(2,6-dioxopiperidin-3-5-(1-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperidin-4-yl)-2-(2,6-dioxopiperidin-3-yl)-6-fluoroisoindoline-1,3-dione;
5-((R)-3-(((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)amino)piperidin-1-yl)-2-(2,6-dioxopiperidin-3-yl)isoindoline-1,3-dione;
5-((S)-3-(((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)amino)piperidin-1-yl)-2-(2,6-dioxopiperidin-3-yl)isoindoline-1,3-dione;
5-(((S)-1-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperidin-3-yl)amino)-2-(2,6-dioxopiperidin-3-yl)isoindoline-1,3-dione;
5-(((R)-1-((1-(2-((S)-4-(4-chlorophenyl)-2,3,9-trimethyl-6H-thieno[3,2-f][1,2,4]triazolo[4,3-a][1,4]diazepin-6-yl)acetyl)piperidin-4-yl)methyl)piperidin-3-yl)amino)-2-(2,6-dioxopiperidin-3-yl)isoindoline-1,3-dione;
5-(4-((1-(4-(5,7-dimethoxy-4-oxo-3,4-dihydroquinazolin-2-yl)phenyl)piperidin-4-yl)methyl)piperazin-1-yl)-2-(2,6-dioxopiperidin-3-yl)isoindoline-1,3-dione;
5-(4-((1-(4-(5,7-dimethoxy-4-oxo-3,4-dihydroquinazolin-2-yl)phenyl)piperidin-4-yl)methyl)piperazin-1-yl)-2-(2,6-dioxopiperidin-3-yl)-6-fluoroisoindoline-1,3-dione;
5-(3-((1-(4-(5,7-dimethoxy-4-oxo-3,4-dihydroquinazolin-2-yl)phenyl)piperidin-4-yl)methyl)-3,6-diazabicyclo[3.1.1]heptan-6-yl)-2-(2,6-dioxopiperidin-3-yl)isoindoline-1,3-dione;
5-(3-((1-(4-(5,7-dimethoxy-4-oxo-3,4-dihydroquinazolin-2-yl)phenyl)piperidin-4-yl)methyl)-3,6-diazabicyclo[3.1.1]heptan-6-yl)-2-(2,6-dioxopiperidin-3-yl)-6-fluoroisoindoline-1,3-dione;
5-(6-((1-(4-(5,7-dimethoxy-4-oxo-3,4-dihydroquinazolin-2-yl)phenyl)piperidin-4-yl)methyl)-3,6-diazabicyclo[3.1.1]heptan-3-yl)-2-(2,6-dioxopiperidin-3-yl)isoindoline-1,3-dione;
5-(6-((1-(4-(5,7-dimethoxy-4-oxo-3,4-dihydroquinazolin-2-yl)phenyl)piperidin-4-yl)methyl)-3,6-diazabicyclo[3.1.1]heptan-3-yl)-2-(2,6-dioxopiperidin-3-yl)-6-fluoroisoindoline-1,3-dione;
5-(3-((1-(4-(5,7-dimethoxy-4-oxo-3,4-dihydroquinazolin-2-yl)phenyl)piperidin-4-yl)methyl)-3,8-diazabicyclo[3.2.1]octan-8-yl)-2-(2,6-dioxopiperidin-3-yl)isoindoline-1,3-dione;
5-(3-((1-(4-(5,7-dimethoxy-4-oxo-3,4-dihydroquinazolin-2-yl)phenyl)piperidin-4-yl)methyl)-3,8-diazabicyclo[3.2.1]octan-8-yl)-2-(2,6-dioxopiperidin-3-yl)isoindoline-1,3-dione;
5-(8-((1-(4-(5,7-dimethoxy-4-oxo-3,4-dihydroquinazolin-2-yl)phenyl)piperidin-4-yl)methyl)-3,8-diazabicyclo[3.2.1]octan-3-yl)-2-(2,6-dioxopiperidin-3-yl)isoindoline-1,3-dione;
5-(8-((1-(4-(5,7-dimethoxy-4-oxo-3,4-dihydroquinazolin-2-yl)phenyl)piperidin-4-yl)methyl)-3,8-diazabicyclo[3.2.1]octan-3-yl)-2-(2,6-dioxopiperidin-3-yl)-6-fluoroisoindoline-1,3-dione;
5-(5-((1-(4-(5,7-dimethoxy-4-oxo-3,4-dihydroquinazolin-2-yl)phenyl)piperidin-4-yl)methyl)-2,5-diazabicyclo[2.2.1]heptan-2-yl)-2-(2,6-dioxopiperidin-3-yl)isoindoline-1,3-dione;
5-(5-((1-(4-(5,7-dimethoxy-4-oxo-3,4-dihydroquinazolin-2-yl)phenyl)piperidin-4-yl)methyl)-2,5-diazabicyclo[2.2.1]heptan-2-yl)-2-(2,6-dioxopiperidin-3-yl)-6-fluoroisoindoline-1,3-dione;
5-(5-((1-(4-(5,7-dimethoxy-4-oxo-3,4-dihydroquinazolin-2-yl)phenyl)piperidin-4-yl)methyl)-2,5-diazabicyclo[2.2.2]octan-2-yl)-2-(2,6-dioxopiperidin-3-yl)isoindoline-1,3-dione;
5-(5-((1-(4-(5,7-dimethoxy-4-oxo-3,4-dihydroquinazolin-2-yl)phenyl)piperidin-4-yl)methyl)-2,5-diazabicyclo[2.2.2]octan-2-yl)-2-(2,6-dioxopiperidin-3-yl)-6-fluoroisoindoline-1,3-dione;
5-(4-((1-(4-(5,7-dimethoxy-4-oxo-3,4-dihydroquinazolin-2-yl)phenyl)piperidin-4-yl)methyl)piperazin-1-yl-2,2,3,3,5,5,6,6-d₈)-2-(2,6-dioxopiperidin-3-yl)isoindoline-1,3-dione;
5-(4-((1-(4-(5,7-dimethoxy-4-oxo-3,4-dihydroquinazolin-2-yl)phenyl)piperidin-4-yl)methyl)piperazin-1-yl-2,2,3,3,5,5,6,6-d₈)-2-(2,6-dioxopiperidin-3-yl)-6-fluoroisoindoline-1,3-dione;
6-(4-((1-(4-(5,7-dimethoxy-4-oxo-3,4-dihydroquinazolin-2-yl)phenyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(4-(5,7-dimethoxy-4-oxo-3,4-dihydroquinazolin-2-yl)phenyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(4-(5,7-dimethoxy-4-oxo-3,4-dihydroquinazolin-2-yl)phenyl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
5-(4-((1-(4-(5,7-dimethoxy-4-oxo-3,4-dihydroquinazolin-2-yl)phenyl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
6-(4-((1-(4-(5,7-dimethoxy-4-oxo-3,4-dihydroquinazolin-2-yl)phenyl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
5-(4-((1-(4-(5,7-dimethoxy-4-oxo-3,4-dihydroquinazolin-2-yl)phenyl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((1-(4-(5,7-dimethoxy-4-oxo-3,4-dihydroquinazolin-2-yl)phenyl)piperidin-4-yl)methyl)-2,6-dimethylpiperazin-1-yl)-2-(2,6-dioxopiperidin-3-yl)isoindoline-1,3-dione;
5-(4-((1-(4-(5,7-dimethoxy-4-oxo-3,4-dihydroquinazolin-2-yl)phenyl)piperidin-4-yl)methyl)-2,6-dimethylpiperazin-1-yl)-2-(2,6-dioxopiperidin-3-yl)-6-fluoroisoindoline-1,3-dione;
5-(4-((1-(4-(5,7-dimethoxy-4-oxo-3,4-dihydroquinazolin-2-yl)phenyl)piperidin-4-yl)methyl)-3,5-dimethylpiperazin-1-yl)-2-(2,6-dioxopiperidin-3-yl)isoindoline-1,3-dione;
5-(4-((1-(4-(5,7-dimethoxy-4-oxo-3,4-dihydroquinazolin-2-yl)phenyl)piperidin-4-yl)methyl)-3,5-dimethylpiperazin-1-yl)-2-(2,6-dioxopiperidin-3-yl)-6-fluoroisoindoline-1,3-dione;
5-(1-((1-(4-(5,7-dimethoxy-4-oxo-3,4-dihydroquinazolin-2-yl)phenyl)piperidin-4-yl)methyl)piperidin-4-yl)-2-(2,6-dioxopiperidin-3-yl)isoindoline-1,3-dione;
5-(1-((1-(4-(5,7-dimethoxy-4-oxo-3,4-dihydroquinazolin-2-yl)phenyl)piperidin-4-yl)methyl)piperidin-4-yl)-2-(2,6-dioxopiperidin-3-yl)-6-fluoroisoindoline-1,3-dione;
5-((R)-3-(((1-(4-(5,7-dimethoxy-4-oxo-3,4-dihydroquinazolin-2-yl)phenyl)piperidin-4-yl)methyl)amino)piperidin-1-yl)-2-(2,6-dioxopiperidin-3-yl)isoindoline-1,3-dione;
5-((S)-3-(((1-(4-(5,7-dimethoxy-4-oxo-3,4-dihydroquinazolin-2-yl)phenyl)piperidin-4-yl)methyl)amino)piperidin-1-yl)-2-(2,6-dioxopiperidin-3-yl)isoindoline-1,3-dione;
5-(((S)-1-((1-(4-(5,7-dimethoxy-4-oxo-3,4-dihydroquinazolin-2-yl)phenyl)piperidin-4-yl)methyl)piperidin-3-yl)amino)-2-(2,6-dioxopiperidin-3-yl)isoindoline-1,3-dione;
5-(((R)-1-((1-(4-(5,7-dimethoxy-4-oxo-3,4-dihydroquinazolin-2-yl)phenyl)piperidin-4-yl)methyl)piperidin-3-yl)amino)-2-(2,6-dioxopiperidin-3-yl)isoindoline-1,3-dione;
N-(2-chloro-6-methylphenyl)-2-((6-(4-((4-(2-(2,6-dioxopiperidin-3-yl)-1,3-dioxoisoindolin-5-yl)piperazin-1-yl)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-6-fluoro-1,3-dioxoisoindolin-5-yl)piperazin-1-yl)methyl)piperidin-1-yl)-2-methylpyrimidin-4-yl)amino)thiazole-5-carboxamide;
N-(2-chloro-6-methylphenyl)-2-((6-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)-2-methylpyrimidin-4-yl)amino)thiazole-5-carboxamide;
N-(2-chloro-6-methylphenyl)-2-((6-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)-2-methylpyrimidin-4-yl)amino)thiazole-5-carboxamide;
N-(2-chloro-6-methylphenyl)-2-((6-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)-2-methylpyrimidin-4-yl)amino)thiazole-5-carboxamide;
N-(2-chloro-6-methylphenyl)-2-((6-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)-2-methylpyrimidin-4-yl)amino)thiazole-5-carboxamide;
N-(2-chloro-6-methylphenyl)-2-((6-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)-2-methylpyrimidin-4-yl)amino)thiazole-5-carboxamide;
N-(2-chloro-6-methylphenyl)-2-((6-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)-2-methylpyrimidin-4-yl)amino)thiazole-5-carboxamide;
N-(2-chloro-6-methylphenyl)-2-((6-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)-2-methylpyrimidin-4-yl)amino)thiazole-5-carboxamide;
N-(2-chloro-6-methylphenyl)-2-((6-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)-2-methylpyrimidin-4-yl)amino)thiazole-5-carboxamide;
N-(2-chloro-6-methylphenyl)-2-((6-(4-((5-(2-(2,6-dioxopiperidin-3-yl)-1,3-dioxoisoindolin-5-yl)-2,5-diazabicyclo[2.2.1]heptan-2-yl)methyl)piperidin-1-yl)-2-methylpyrimidin-4-yl)amino)thiazole-5-carboxamide;
N-(2-chloro-6-methylphenyl)-2-((6-(4-((5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)-2-methylpyrimidin-4-yl)amino)thiazole-5-carboxamide;
N-(2-chloro-6-methylphenyl)-2-((6-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)-2-methylpyrimidin-4-yl)amino)thiazole-5-carboxamide;
N-(2-chloro-6-methylphenyl)-2-((6-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-1,3-dioxoisoindolin-5-yl)-3,5-dimethylpiperazin-1-yl)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-1,3-dioxoisoindolin-5-yl)-2,6-dimethylpiperazin-1-yl)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-1,3-dioxoisoindolin-5-yl)piperidin-1-yl)methyl)piperidin-1-yl)-2-methylpyrimidin-4-yl)amino)thiazole-5-carboxamide;
N-(2-chloro-6-methylphenyl)-2-((6-(4-((4-(2-(2,6-dioxopiperidin-3-yl)-6-fluoro-1,3-dioxoisoindolin-5-yl)piperidin-1-yl)methyl)piperidin-1-yl)-2-methylpyrimidin-4-yl)amino)thiazole-5-carboxamide;
N-(2-chloro-6-methylphenyl)-2-((6-(4-((((3R)-1-(2-(2,6-dioxopiperidin-3-yl)-1,3-dioxoisoindolin-5-yl)piperidin-3-yl)amino)methyl)piperidin-1-yl)-2-methylpyrimidin-4-yl)amino)thiazole-5-carboxamide;
N-(2-chloro-6-methylphenyl)-2-((6-(4-((((3S)-1-(2-(2,6-dioxopiperidin-3-yl)-1,3-dioxoisoindolin-5-yl)piperidin-3-yl)amino)methyl)piperidin-1-yl)-2-methylpyrimidin-4-yl)amino)thiazole-5-carboxamide;
N-(2-chloro-6-methylphenyl)-2-((6-(4-(((3S)-3-((2-(2,6-dioxopiperidin-3-yl)-1,3-dioxoisoindolin-5-yl)amino)piperidin-1-yl)methyl)piperidin-1-yl)-2-methylpyrimidin-4-yl)amino)thiazole-5-carboxamide;
N-(2-chloro-6-methylphenyl)-2-((6-(4-(((3R)-3-((2-(2,6-dioxopiperidin-3-yl)-1,3-dioxoisoindolin-5-yl)amino)piperidin-1-yl)methyl)piperidin-1-yl)-2-methylpyrimidin-4-yl)amino)thiazole-5-carboxamide;
4-((2,4-dichloro-5-methoxyphenyl)amino)-7-(3-(4-((4-(2-(2,6-dioxopiperidin-3-yl)-1,3-dioxoisoindolin-5-yl)piperazin-1-yl)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-6-fluoro-1,3-dioxoisoindolin-5-yl)piperazin-1-yl)methyl)piperidin-1-yl)propoxy)-6-methoxyquinoline-3-carbonitrile;
4-((2,4-dichloro-5-methoxyphenyl)amino)-7-(3-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)propoxy)-6-methoxyquinoline-3-carbonitrile;
4-((2,4-dichloro-5-methoxyphenyl)amino)-7-(3-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)propoxy)-6-methoxyquinoline-3-carbonitrile;
4-((2,4-dichloro-5-methoxyphenyl)amino)-7-(3-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)propoxy)-6-methoxyquinoline-3-carbonitrile;
4-((2,4-dichloro-5-methoxyphenyl)amino)-7-(3-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)propoxy)-6-methoxyquinoline-3-carbonitrile;
4-((2,4-dichloro-5-methoxyphenyl)amino)-7-(3-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)propoxy)-6-methoxyquinoline-3-carbonitrile;
4-((2,4-dichloro-5-methoxyphenyl)amino)-7-(3-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)propoxy)-6-methoxyquinoline-3-carbonitrile;
4-((2,4-dichloro-5-methoxyphenyl)amino)-7-(3-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)propoxy)-6-methoxyquinoline-3-carbonitrile;
4-((2,4-dichloro-5-methoxyphenyl)amino)-7-(3-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)propoxy)-6-methoxyquinoline-3-carbonitrile;
4-((2,4-dichloro-5-methoxyphenyl)amino)-7-(3-(4-((5-(2-(2,6-dioxopiperidin-3-yl)-1,3-dioxoisoindolin-5-yl)-2,5-diazabicyclo[2.2.1]heptan-2-yl)methyl)piperidin-1-yl)propoxy)-6-methoxyquinoline-3-carbonitrile;
4-((2,4-dichloro-5-methoxyphenyl)amino)-7-(3-(4-((5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)propoxy)-6-methoxyquinoline-3-carbonitrile;
4-((2,4-dichloro-5-methoxyphenyl)amino)-7-(3-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)propoxy)-6-methoxyquinoline-3-carbonitrile;
4-((2,4-dichloro-5-methoxyphenyl)amino)-7-(3-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-1,3-dioxoisoindolin-5-yl)-3,5-dimethylpiperazin-1-yl)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-1,3-dioxoisoindolin-5-yl)-2,6-dimethylpiperazin-1-yl)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-1,3-dioxoisoindolin-5-yl)piperidin-1-yl)methyl)piperidin-1-yl)propoxy)-6-methoxyquinoline-3-carbonitrile;
4-((2,4-dichloro-5-methoxyphenyl)amino)-7-(3-(4-((4-(2-(2,6-dioxopiperidin-3-yl)-6-fluoro-1,3-dioxoisoindolin-5-yl)piperidin-1-yl)methyl)piperidin-1-yl)propoxy)-6-methoxyquinoline-3-carbonitrile;
4-((2,4-dichloro-5-methoxyphenyl)amino)-7-(3-(4-((((3R)-1-(2-(2,6-dioxopiperidin-3-yl)-1,3-dioxoisoindolin-5-yl)piperidin-3-yl)amino)methyl)piperidin-1-yl)propoxy)-6-methoxyquinoline-3-carbonitrile;
4-((2,4-dichloro-5-methoxyphenyl)amino)-7-(3-(4-((((3S)-1-(2-(2,6-dioxopiperidin-3-yl)-1,3-dioxoisoindolin-5-yl)piperidin-3-yl)amino)methyl)piperidin-1-yl)propoxy)-6-methoxyquinoline-3-carbonitrile;
4-((2,4-dichloro-5-methoxyphenyl)amino)-7-(3-(4-(((3S)-3-((2-(2,6-dioxopiperidin-3-yl)-1,3-dioxoisoindolin-5-yl)amino)piperidin-1-yl)methyl)piperidin-1-yl)propoxy)-6-methoxyquinoline-3-carbonitrile;
4-((2,4-dichloro-5-methoxyphenyl)amino)-7-(3-(4-(((3R)-3-((2-(2,6-dioxopiperidin-3-yl)-1,3-dioxoisoindolin-5-yl)amino)piperidin-1-yl)methyl)piperidin-1-yl)propoxy)-6-methoxyquinoline-3-carbonitrile;
4-((4-((4-(2-(2,6-dioxopiperidin-3-yl)-1,3-dioxoisoindolin-5-yl)piperazin-1-yl)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-6-fluoro-1,3-dioxoisoindolin-5-yl)piperazin-1-yl)methyl)piperidin-1-yl)methyl)-N-(4-methyl-3-((4-(pyridin-3-yl)pyrimidin-2-yl)amino)phenyl)benzamide;
4-((4-((6-(2-(2,6-dioxopiperidin-3-yl)-1,3-dioxoisoindolin-5-yl)-3,6-diazabicyclo[3.1.1]heptan-3-yl)methyl)piperidin-1-yl)methyl)-N-(4-methyl-3-((4-(pyridin-3-yl)pyrimidin-2-yl)amino)phenyl)benzamide;
4-((4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)methyl)-N-(4-methyl-3-((4-(pyridin-3-yl)pyrimidin-2-yl)amino)phenyl)benzamide;
4-((4-((3-(2-(2,6-dioxopiperidin-3-yl)-1,3-dioxoisoindolin-5-yl)-3,6-diazabicyclo[3.1.1]heptan-6-yl)methyl)piperidin-1-yl)methyl)-N-(4-methyl-3-((4-(pyridin-3-yl)pyrimidin-2-yl)amino)phenyl)benzamide;
4-((4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)methyl)-N-(4-methyl-3-((4-(pyridin-3-yl)pyrimidin-2-yl)amino)phenyl)benzamide;
4-((4-((8-(2-(2,6-dioxopiperidin-3-yl)-1,3-dioxoisoindolin-5-yl)-3,8-diazabicyclo[3.2.1]octan-3-yl)methyl)piperidin-1-yl)methyl)-N-(4-methyl-3-((4-(pyridin-3-yl)pyrimidin-2-yl)amino)phenyl)benzamide;
4-((4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)methyl)-N-(4-methyl-3-((4-(pyridin-3-yl)pyrimidin-2-yl)amino)phenyl)benzamide;
4-((4-((3-(2-(2,6-dioxopiperidin-3-yl)-1,3-dioxoisoindolin-5-yl)-3,8-diazabicyclo[3.2.1]octan-8-yl)methyl)piperidin-1-yl)methyl)-N-(4-methyl-3-((4-(pyridin-3-yl)pyrimidin-2-yl)amino)phenyl)benzamide;
4-((4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)methyl)-N-(4-methyl-3-((4-(pyridin-3-yl)pyrimidin-2-yl)amino)phenyl)benzamide;
4-((4-((5-(2-(2,6-dioxopiperidin-3-yl)-1,3-dioxoisoindolin-5-yl)-2,5-diazabicyclo[2.2.1]heptan-2-yl)methyl)piperidin-1-yl)methyl)-N-(4-methyl-3-((4-(pyridin-3-yl)pyrimidin-2-yl)amino)phenyl)benzamide;
4-((4-((5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)methyl)-N-(4-methyl-3-((4-(pyridin-3-yl)pyrimidin-2-yl)amino)phenyl)benzamide;
4-((4-((5-(2-(2,6-dioxopiperidin-3-yl)-1,3-dioxoisoindolin-5-yl)-2,5-diazabicyclo[2.2.2]octan-2-yl)methyl)piperidin-1-yl)methyl)-N-(4-methyl-3-((4-(pyridin-3-yl)pyrimidin-2-yl)amino)phenyl)benzamide;
4-((4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-1,3-dioxoisoindolin-5-yl)-3,5-dimethylpiperazin-1-yl)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-6-fluoro-1,3-dioxoisoindolin-5-yl)-3,5-dimethylpiperazin-1-yl)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-1,3-dioxoisoindolin-5-yl)-2,6-dimethylpiperazin-1-yl)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-6-fluoro-1,3-dioxoisoindolin-5-yl)-2,6-dimethylpiperazin-1-yl)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-1,3-dioxoisoindolin-5-yl)piperidin-1-yl)methyl)piperidin-1-yl)methyl)-N-(4-methyl-3-((4-(pyridin-3-yl)pyrimidin-2-yl)amino)phenyl)benzamide;
4-((4-((4-(2-(2,6-dioxopiperidin-3-yl)-6-fluoro-1,3-dioxoisoindolin-5-yl)piperidin-1-yl)methyl)piperidin-1-yl)methyl)-N-(4-methyl-3-((4-(pyridin-3-yl)pyrimidin-2-yl)amino)phenyl)benzamide;
4-((4-((((3R)-1-(2-(2,6-dioxopiperidin-3-yl)-1,3-dioxoisoindolin-5-yl)piperidin-3-yl)amino)methyl)piperidin-1-yl)methyl)-N-(4-methyl-3-((4-(pyridin-3-yl)pyrimidin-2-yl)amino)phenyl)benzamide;
4-((4-((((3S)-1-(2-(2,6-dioxopiperidin-3-yl)-1,3-dioxoisoindolin-5-yl)piperidin-3-yl)amino)methyl)piperidin-1-yl)methyl)-N-(4-methyl-3-((4-(pyridin-3-yl)pyrimidin-2-yl)amino)phenyl)benzamide;
4-((4-(((3S)-3-((2-(2,6-dioxopiperidin-3-yl)-1,3-dioxoisoindolin-5-yl)amino)piperidin-1-yl)methyl)piperidin-1-yl)methyl)-N-(4-methyl-3-((4-(pyridin-3-yl)pyrimidin-2-yl)amino)phenyl)benzamide;
4-((4-(((3R)-3-((2-(2,6-dioxopiperidin-3-yl)-1,3-dioxoisoindolin-5-yl)amino)piperidin-1-yl)methyl)piperidin-1-yl)methyl)-N-(4-methyl-3-((4-(pyridin-3-yl)pyrimidin-2-yl)amino)phenyl)benzamide;
2-((2-((4-(4-((4-(2-(2,6-dioxopiperidin-3-yl)-1,3-dioxoisoindolin-5-yl)piperazin-1-yl)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-6-fluoro-1,3-dioxoisoindolin-5-yl)piperazin-1-yl)methyl)piperidin-1-yl)-2-methoxyphenyl)amino)-5-(trifluoromethyl)pyridin-4-yl)amino)-N-methylbenzamide;
2-((2-((4-(4-((6-(2-(2,6-dioxopiperidin-3-yl)-1,3-dioxoisoindolin-5-yl)-3,6-diazabicyclo[3.1.1]heptan-3-yl)methyl)piperidin-1-yl)-2-methoxyphenyl)amino)-5-(trifluoromethyl)pyridin-4-yl)amino)-N-methylbenzamide;
2-((2-((4-(4-((6-(2-(2,6-dioxopiperidin-3-yl)-6-fluoro-1,3-dioxoisoindolin-5-yl)-3,6-diazabicyclo[3.1.1]heptan-3-yl)methyl)piperidin-1-yl)-2-methoxyphenyl)amino)-5-(trifluoromethyl)pyridin-4-yl)amino)-N-methylbenzamide;
2-((2-((4-(4-((3-(2-(2,6-dioxopiperidin-3-yl)-1,3-dioxoisoindolin-5-yl)-3,6-diazabicyclo[3.1.1]heptan-6-yl)methyl)piperidin-1-yl)-2-methoxyphenyl)amino)-5-(trifluoromethyl)pyridin-4-yl)amino)-N-methylbenzamide;
2-((2-((4-(4-((3-(2-(2,6-dioxopiperidin-3-yl)-6-fluoro-1,3-dioxoisoindolin-5-yl)-3,6-diazabicyclo[3.1.1]heptan-6-yl)methyl)piperidin-1-yl)-2-methoxyphenyl)amino)-5-(trifluoromethyl)pyridin-4-yl)amino)-N-methylbenzamide;
2-((2-((4-(4-((8-(2-(2,6-dioxopiperidin-3-yl)-1,3-dioxoisoindolin-5-yl)-3,8-diazabicyclo[3.2.1]octan-3-yl)methyl)piperidin-1-yl)-2-methoxyphenyl)amino)-5-(trifluoromethyl)pyridin-4-yl)amino)-N-methylbenzamide;
2-((2-((4-(4-((8-(2-(2,6-dioxopiperidin-3-yl)-6-fluoro-1,3-dioxoisoindolin-5-yl)-3,8-diazabicyclo[3.2.1]octan-3-yl)methyl)piperidin-1-yl)-2-methoxyphenyl)amino)-5-(trifluoromethyl)pyridin-4-yl)amino)-N-methylbenzamide;
2-((2-((4-(4-((3-(2-(2,6-dioxopiperidin-3-yl)-1,3-dioxoisoindolin-5-yl)-3,8-diazabicyclo[3.2.1]octan-8-yl)methyl)piperidin-1-yl)-2-methoxyphenyl)amino)-5-(trifluoromethyl)pyridin-4-yl)amino)-N-methylbenzamide;
2-((2-((4-(4-((3-(2-(2,6-dioxopiperidin-3-yl)-6-fluoro-1,3-dioxoisoindolin-5-yl)-3,8-diazabicyclo[3.2.1]octan-8-yl)methyl)piperidin-1-yl)-2-methoxyphenyl)amino)-5-(trifluoromethyl)pyridin-4-yl)amino)-N-methylbenzamide;
2-((2-((4-(4-((5-(2-(2,6-dioxopiperidin-3-yl)-1,3-dioxoisoindolin-5-yl)-2,5-diazabicyclo[2.2.1]heptan-2-yl)methyl)piperidin-1-yl)-2-methoxyphenyl)amino)-5-(trifluoromethyl)pyridin-4-yl)amino)-N-methylbenzamide;
2-((2-((4-(4-((5-(2-(2,6-dioxopiperidin-3-yl)-6-fluoro-1,3-dioxoisoindolin-5-yl)-2,5-diazabicyclo[2.2.1]heptan-2-yl)methyl)piperidin-1-yl)-2-methoxyphenyl)amino)-5-(trifluoromethyl)pyridin-4-yl)amino)-N-methylbenzamide;
2-((2-((4-(4-((5-(2-(2,6-dioxopiperidin-3-yl)-1,3-dioxoisoindolin-5-yl)-2,5-diazabicyclo[2.2.2]octan-2-yl)methyl)piperidin-1-yl)-2-methoxyphenyl)amino)-5-(trifluoromethyl)pyridin-4-yl)amino)-N-methylbenzamide;
2-((2-((4-(4-((5-(2-(2,6-dioxopiperidin-3-yl)-6-fluoro-1,3-dioxoisoindolin-5-yl)-2,5-diazabicyclo[2.2.2]octan-2-yl)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-1,3-dioxoisoindolin-5-yl)piperazin-1-yl-2,2,3,3,5,5,6,6-d₈)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-6-fluoro-1,3-dioxoisoindolin-5-yl)piperazin-1-yl-2,2,3,3,5,5,6,6-d₈)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-7-fluoro-1,3-dioxoisoindolin-5-yl)piperazin-1-yl-2,2,3,3,5,5,6,6-d₈)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-4-fluoro-1,3-dioxoisoindolin-5-yl)piperazin-1-yl-2,2,3,3,5,5,6,6-d₈)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-4,6-difluoro-1,3-dioxoisoindolin-5-yl)piperazin-1-yl-2,2,3,3,5,5,6,6-d₈)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-4,7-difluoro-1,3-dioxoisoindolin-5-yl)piperazin-1-yl-2,2,3,3,5,5,6,6-d₈)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-6,7-difluoro-1,3-dioxoisoindolin-5-yl)piperazin-1-yl-2,2,3,3,5,5,6,6-d₈)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-4,6,7-trifluoro-1,3-dioxoisoindolin-5-yl)piperazin-1-yl-2,2,3,3,5,5,6,6-d₈)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-1,3-dioxoisoindolin-5-yl)-3,5-dimethylpiperazin-1-yl)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-6-fluoro-1,3-dioxoisoindolin-5-yl)-3,5-dimethylpiperazin-1-yl)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-1,3-dioxoisoindolin-5-yl)-2,6-dimethylpiperazin-1-yl)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-6-fluoro-1,3-dioxoisoindolin-5-yl)-2,6-dimethylpiperazin-1-yl)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-1,3-dioxoisoindolin-5-yl)piperidin-1-yl)methyl)piperidin-1-yl)-2-methoxyphenyl)amino)-5-(trifluoromethyl)pyridin-4-yl)amino)-N-methylbenzamide;
2-((2-((4-(4-((4-(2-(2,6-dioxopiperidin-3-yl)-6-fluoro-1,3-dioxoisoindolin-5-yl)piperidin-1-yl)methyl)piperidin-1-yl)-2-methoxyphenyl)amino)-5-(trifluoromethyl)pyridin-4-yl)amino)-N-methylbenzamide;
2-((2-((4-(4-((((3R)-1-(2-(2,6-dioxopiperidin-3-yl)-1,3-dioxoisoindolin-5-yl)piperidin-3-yl)amino)methyl)piperidin-1-yl)-2-methoxyphenyl)amino)-5-(trifluoromethyl)pyridin-4-yl)amino)-N-methylbenzamide;
2-((2-((4-(4-((((3S)-1-(2-(2,6-dioxopiperidin-3-yl)-1,3-dioxoisoindolin-5-yl)piperidin-3-yl)amino)methyl)piperidin-1-yl)-2-methoxyphenyl)amino)-5-(trifluoromethyl)pyridin-4-yl)amino)-N-methylbenzamide;
2-((2-((4-(4-(((3S)-3-((2-(2,6-dioxopiperidin-3-yl)-1,3-dioxoisoindolin-5-yl)amino)piperidin-1-yl)methyl)piperidin-1-yl)-2-methoxyphenyl)amino)-5-(trifluoromethyl)pyridin-4-yl)amino)-N-methylbenzamide;
2-((2-((4-(4-(((3R)-3-((2-(2,6-dioxopiperidin-3-yl)-1,3-dioxoisoindolin-5-yl)amino)piperidin-1-yl)methyl)piperidin-1-yl)-2-methoxyphenyl)amino)-5-(trifluoromethyl)pyridin-4-yl)amino)-N-methylbenzamide;
5-(4-((1-(2-(4-(4-chloro-1,2-diphenylbut-1-en-1-yl)phenoxy)ethyl)piperidin-4-yl)methyl)piperazin-1-yl)-2-(2,6-dioxopiperidin-3-yl)isoindoline-1,3-dione;
5-(4-((1-(2-(4-(4-chloro-1-(4-hydroxyphenyl)-2-phenylbut-1-en-1-yl)phenoxy)ethyl)piperidin-4-yl)methyl)piperazin-1-yl)-2-(2,6-dioxopiperidin-3-yl)isoindoline-1,3-dione;
5-(4-((1-(2-(4-(4-chloro-1,2-bis(4-hydroxyphenyl)but-1-en-1-yl)phenoxy)ethyl)piperidin-4-yl)methyl)piperazin-1-yl)-2-(2,6-dioxopiperidin-3-yl)isoindoline-1,3-dione;
5-(4-((1-(2-(4-(4-chloro-1,2-diphenylbut-1-en-1-yl)phenoxy)ethyl)piperidin-4-yl)methyl)piperazin-1-yl)-2-(2,6-dioxopiperidin-3-yl)-6-fluoroisoindoline-1,3-dione;
5-(4-((1-(2-(4-(4-chloro-1-(4-hydroxyphenyl)-2-phenylbut-1-en-1-yl)phenoxy)ethyl)piperidin-4-yl)methyl)piperazin-1-yl)-2-(2,6-dioxopiperidin-3-yl)-6-fluoroisoindoline-1,3-dione;
5-(4-((1-(2-(4-(4-chloro-1,2-bis(4-hydroxyphenyl)but-1-en-1-yl)phenoxy)ethyl)piperidin-4-yl)methyl)piperazin-1-yl)-2-(2,6-dioxopiperidin-3-yl)-6-fluoroisoindoline-1,3-dione;
5-(3-((1-(2-(4-(4-chloro-1,2-diphenylbut-1-en-1-yl)phenoxy)ethyl)piperidin-4-yl)methyl)-3,6-diazabicyclo[3.1.1]heptan-6-yl)-2-(2,6-dioxopiperidin-3-yl)isoindoline-1,3-dione;
5-(3-((1-(2-(4-(4-chloro-1-(4-hydroxyphenyl)-2-phenylbut-1-en-1-yl)phenoxy)ethyl)piperidin-4-yl)methyl)-3,6-diazabicyclo[3.1.1]heptan-6-yl)-2-(2,6-dioxopiperidin-3-yl)isoindoline-1,3-dione;
5-(3-((1-(2-(4-(4-chloro-1,2-bis(4-hydroxyphenyl)but-1-en-1-yl)phenoxy)ethyl)piperidin-4-yl)methyl)-3,6-diazabicyclo[3.1.1]heptan-6-yl)-2-(2,6-dioxopiperidin-3-yl)isoindoline-1,3-dione;
5-(3-((1-(2-(4-(4-chloro-1,2-diphenylbut-1-en-1-yl)phenoxy)ethyl)piperidin-4-yl)methyl)-3,6-diazabicyclo[3.1.1]heptan-6-yl)-2-(2,6-dioxopiperidin-3-yl)-6-fluoroisoindoline-1,3-dione;
5-(3-((1-(2-(4-(4-chloro-1-(4-hydroxyphenyl)-2-phenylbut-1-en-1-yl)phenoxy)ethyl)piperidin-4-yl)methyl)-3,6-diazabicyclo[3.1.1]heptan-6-yl)-2-(2,6-dioxopiperidin-3-yl)-6-fluoroisoindoline-1,3-dione;
5-(3-((1-(2-(4-(4-chloro-1,2-bis(4-hydroxyphenyl)but-1-en-1-yl)phenoxy)ethyl)piperidin-4-yl)methyl)-3,6-diazabicyclo[3.1.1]heptan-6-yl)-2-(2,6-dioxopiperidin-3-yl)-6-fluoroisoindoline-1,3-dione;
5-(6-((1-(2-(4-(4-chloro-1,2-diphenylbut-1-en-1-yl)phenoxy)ethyl)piperidin-4-yl)methyl)-3,6-diazabicyclo[3.1.1]heptan-3-yl)-2-(2,6-dioxopiperidin-3-yl)isoindoline-1,3-dione;
5-(6-((1-(2-(4-(4-chloro-1-(4-hydroxyphenyl)-2-phenylbut-1-en-1-yl)phenoxy)ethyl)piperidin-4-yl)methyl)-3,6-diazabicyclo[3.1.1]heptan-3-yl)-2-(2,6-dioxopiperidin-3-yl)isoindoline-1,3-dione;
5-(6-((1-(2-(4-(4-chloro-1,2-bis(4-hydroxyphenyl)but-1-en-1-yl)phenoxy)ethyl)piperidin-4-yl)methyl)-3,6-diazabicyclo[3.1.1]heptan-3-yl)-2-(2,6-dioxopiperidin-3-yl)isoindoline-1,3-dione;
5-(6-((1-(2-(4-(4-chloro-1,2-diphenylbut-1-en-1-yl)phenoxy)ethyl)piperidin-4-yl)methyl)-3,6-diazabicyclo[3.1.1]heptan-3-yl)-2-(2,6-dioxopiperidin-3-yl)-6-fluoroisoindoline-1,3-dione;
5-(6-((1-(2-(4-(4-chloro-1-(4-hydroxyphenyl)-2-phenylbut-1-en-1-yl)phenoxy)ethyl)piperidin-4-yl)methyl)-3,6-diazabicyclo[3.1.1]heptan-3-yl)-2-(2,6-dioxopiperidin-3-yl)-6-fluoroisoindoline-1,3-dione;
5-(6-((1-(2-(4-(4-chloro-1,2-bis(4-hydroxyphenyl)but-1-en-1-yl)phenoxy)ethyl)piperidin-4-yl)methyl)-3,6-diazabicyclo[3.1.1]heptan-3-yl)-2-(2,6-dioxopiperidin-3-yl)-6-fluoroisoindoline-1,3-dione;
5-(3-((1-(2-(4-(4-chloro-1,2-diphenylbut-1-en-1-yl)phenoxy)ethyl)piperidin-4-yl)methyl)-3,8-diazabicyclo[3.2.1]octan-8-yl)-2-(2,6-dioxopiperidin-3-yl)isoindoline-1,3-dione;
5-(3-((1-(2-(4-(4-chloro-1-(4-hydroxyphenyl)-2-phenylbut-1-en-1-yl)phenoxy)ethyl)piperidin-4-yl)methyl)-3,8-diazabicyclo[3.2.1]octan-8-yl)-2-(2,6-dioxopiperidin-3-yl)isoindoline-1,3-dione;
5-(3-((1-(2-(4-(4-chloro-1,2-bis(4-hydroxyphenyl)but-1-en-1-yl)phenoxy)ethyl)piperidin-4-yl)methyl)-3,8-diazabicyclo[3.2.1]octan-8-yl)-2-(2,6-dioxopiperidin-3-yl)isoindoline-1,3-dione;
5-(3-((1-(2-(4-(4-chloro-1,2-diphenylbut-1-en-1-yl)phenoxy)ethyl)piperidin-4-yl)methyl)-3,8-diazabicyclo[3.2.1]octan-8-yl)-2-(2,6-dioxopiperidin-3-yl)-6-fluoroisoindoline-1,3-dione;
5-(3-((1-(2-(4-(4-chloro-1-(4-hydroxyphenyl)-2-phenylbut-1-en-1-yl)phenoxy)ethyl)piperidin-4-yl)methyl)-3,8-diazabicyclo[3.2.1]octan-8-yl)-2-(2,6-dioxopiperidin-3-yl)-6-fluoroisoindoline-1,3-dione;
5-(3-((1-(2-(4-(4-chloro-1,2-bis(4-hydroxyphenyl)but-1-en-1-yl)phenoxy)ethyl)piperidin-4-yl)methyl)-3,8-diazabicyclo[3.2.1]octan-8-yl)-2-(2,6-dioxopiperidin-3-yl)-6-fluoroisoindoline-1,3-dione;
5-(8-((1-(2-(4-(4-chloro-1,2-diphenylbut-1-en-1-yl)phenoxy)ethyl)piperidin-4-yl)methyl)-3,8-diazabicyclo[3.2.1]octan-3-yl)-2-(2,6-dioxopiperidin-3-yl)isoindoline-1,3-dione;
5-(8-((1-(2-(4-(4-chloro-1-(4-hydroxyphenyl)-2-phenylbut-1-en-1-yl)phenoxy)ethyl)piperidin-4-yl)methyl)-3,8-diazabicyclo[3.2.1]octan-3-yl)-2-(2,6-dioxopiperidin-3-yl)isoindoline-1,3-dione;
5-(8-((1-(2-(4-(4-chloro-1,2-bis(4-hydroxyphenyl)but-1-en-1-yl)phenoxy)ethyl)piperidin-4-yl)methyl)-3,8-diazabicyclo[3.2.1]octan-3-yl)-2-(2,6-dioxopiperidin-3-yl)isoindoline-1,3-dione;
5-(8-((1-(2-(4-(4-chloro-1,2-diphenylbut-1-en-1-yl)phenoxy)ethyl)piperidin-4-yl)methyl)-3,8-diazabicyclo[3.2.1]octan-3-yl)-2-(2,6-dioxopiperidin-3-yl)-6-fluoroisoindoline-1,3-dione;
5-(8-((1-(2-(4-(4-chloro-1-(4-hydroxyphenyl)-2-phenylbut-1-en-1-yl)phenoxy)ethyl)piperidin-4-yl)methyl)-3,8-diazabicyclo[3.2.1]octan-3-yl)-2-(2,6-dioxopiperidin-3-yl)-6-fluoroisoindoline-1,3-dione;
5-(8-((1-(2-(4-(4-chloro-1,2-bis(4-hydroxyphenyl)but-1-en-1-yl)phenoxy)ethyl)piperidin-4-yl)methyl)-3,8-diazabicyclo[3.2.1]octan-3-yl)-2-(2,6-dioxopiperidin-3-yl)-6-fluoroisoindoline-1,3-dione;
5-(5-((1-(2-(4-(4-chloro-1,2-diphenylbut-1-en-1-yl)phenoxy)ethyl)piperidin-4-yl)methyl)-2,5-diazabicyclo[2.2.1]heptan-2-yl)-2-(2,6-dioxopiperidin-3-yl)isoindoline-1,3-dione;
5-(5-((1-(2-(4-(4-chloro-1-(4-hydroxyphenyl)-2-phenylbut-1-en-1-yl)phenoxy)ethyl)piperidin-4-yl)methyl)-2,5-diazabicyclo[2.2.1]heptan-2-yl)-2-(2,6-dioxopiperidin-3-yl)isoindoline-1,3-dione;
5-(5-((1-(2-(4-(4-chloro-1,2-bis(4-hydroxyphenyl)but-1-en-1-yl)phenoxy)ethyl)piperidin-4-yl)methyl)-2,5-diazabicyclo[2.2.1]heptan-2-yl)-2-(2,6-dioxopiperidin-3-yl)isoindoline-1,3-dione;
5-(5-((1-(2-(4-(4-chloro-1,2-diphenylbut-1-en-1-yl)phenoxy)ethyl)piperidin-4-yl)methyl)-2,5-diazabicyclo[2.2.1]heptan-2-yl)-2-(2,6-dioxopiperidin-3-yl)-6-fluoroisoindoline-1,3-dione;
5-(5-((1-(2-(4-(4-chloro-1-(4-hydroxyphenyl)-2-phenylbut-1-en-1-yl)phenoxy)ethyl)piperidin-4-yl)methyl)-2,5-diazabicyclo[2.2.1]heptan-2-yl)-2-(2,6-dioxopiperidin-3-yl)-6-fluoroisoindoline-1,3-dione;
5-(5-((1-(2-(4-(4-chloro-1,2-bis(4-hydroxyphenyl)but-1-en-1-yl)phenoxy)ethyl)piperidin-4-yl)methyl)-2,5-diazabicyclo[2.2.1]heptan-2-yl)-2-(2,6-dioxopiperidin-3-yl)-6-fluoroisoindoline-1,3-dione;
5-(5-((1-(2-(4-(4-chloro-1,2-diphenylbut-1-en-1-yl)phenoxy)ethyl)piperidin-4-yl)methyl)-2,5-diazabicyclo[2.2.2]octan-2-yl)-2-(2,6-dioxopiperidin-3-yl)isoindoline-1,3-dione;
5-(5-((1-(2-(4-(4-chloro-1-(4-hydroxyphenyl)-2-phenylbut-1-en-1-yl)phenoxy)ethyl)piperidin-4-yl)methyl)-2,5-diazabicyclo[2.2.2]octan-2-yl)-2-(2,6-dioxopiperidin-3-yl)isoindoline-1,3-dione;
5-(5-((1-(2-(4-(4-chloro-1,2-bis(4-hydroxyphenyl)but-1-en-1-yl)phenoxy)ethyl)piperidin-4-yl)methyl)-2,5-diazabicyclo[2.2.2]octan-2-yl)-2-(2,6-dioxopiperidin-3-yl)isoindoline-1,3-dione;
5-(5-((1-(2-(4-(4-chloro-1,2-diphenylbut-1-en-1-yl)phenoxy)ethyl)piperidin-4-yl)methyl)-2,5-diazabicyclo[2.2.2]octan-2-yl)-2-(2,6-dioxopiperidin-3-yl)-6-fluoroisoindoline-1,3-dione;
5-(5-((1-(2-(4-(4-chloro-1-(4-hydroxyphenyl)-2-phenylbut-1-en-1-yl)phenoxy)ethyl)piperidin-4-yl)methyl)-2,5-diazabicyclo[2.2.2]octan-2-yl)-2-(2,6-dioxopiperidin-3-yl)-6-fluoroisoindoline-1,3-dione;
5-(5-((1-(2-(4-(4-chloro-1,2-bis(4-hydroxyphenyl)but-1-en-1-yl)phenoxy)ethyl)piperidin-4-yl)methyl)-2,5-diazabicyclo[2.2.2]octan-2-yl)-2-(2,6-dioxopiperidin-3-yl)-6-fluoroisoindoline-1,3-dione;
5-(4-((1-(2-(4-(4-chloro-1,2-diphenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)isoindoline-1,3-dione;
5-(4-((1-(2-(4-(4-chloro-1-(4-hydroxyphenyl)-2-phenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)isoindoline-1,3-dione;
5-(4-((1-(2-(4-(4-chloro-1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)isoindoline-1,3-dione;
5-(4-((1-(2-(4-(4-chloro-1,2-diphenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-6-fluoroisoindoline-1,3-dione;
5-(4-((1-(2-(4-(4-chloro-1-(4-hydroxyphenyl)-2-phenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-6-fluoroisoindoline-1,3-dione;
5-(4-((1-(2-(4-(4-chloro-1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-6-fluoroisoindoline-1,3-dione;
6-(4-((1-(2-(4-(4-chloro-1,2-diphenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
6-(4-((1-(2-(4-(4-chloro-1-(4-hydroxyphenyl)-2-phenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
6-(4-((1-(2-(4-(4-chloro-1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(2-(4-(4-chloro-1,2-diphenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(2-(4-(4-chloro-1-(4-hydroxyphenyl)-2-phenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(2-(4-(4-chloro-1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(2-(4-(4-chloro-1,2-diphenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
5-(4-((1-(2-(4-(4-chloro-1-(4-hydroxyphenyl)-2-phenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
5-(4-((1-(2-(4-(4-chloro-1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
5-(4-((1-(2-(4-(4-chloro-1,2-diphenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
5-(4-((1-(2-(4-(4-chloro-1-(4-hydroxyphenyl)-2-phenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
5-(4-((1-(2-(4-(4-chloro-1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
6-(4-((1-(2-(4-(4-chloro-1,2-diphenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
6-(4-((1-(2-(4-(4-chloro-1-(4-hydroxyphenyl)-2-phenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
6-(4-((1-(2-(4-(4-chloro-1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
5-(4-((1-(2-(4-(4-chloro-1,2-diphenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((1-(2-(4-(4-chloro-1-(4-hydroxyphenyl)-2-phenylbut-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((1-(2-(4-(4-chloro-1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((1-(2-(4-(4-chloro-1,2-diphenylbut-1-en-1-yl)phenoxy)ethyl)piperidin-4-yl)methyl)-2,6-dimethylpiperazin-1-yl)-2-(2,6-dioxopiperidin-3-yl)isoindoline-1,3-dione;
5-(4-((1-(2-(4-(4-chloro-1-(4-hydroxyphenyl)-2-phenylbut-1-en-1-yl)phenoxy)ethyl)piperidin-4-yl)methyl)-2,6-dimethylpiperazin-1-yl)-2-(2,6-dioxopiperidin-3-yl)isoindoline-1,3-dione;
5-(4-((1-(2-(4-(4-chloro-1,2-bis(4-hydroxyphenyl)but-1-en-1-yl)phenoxy)ethyl)piperidin-4-yl)methyl)-2,6-dimethylpiperazin-1-yl)-2-(2,6-dioxopiperidin-3-yl)isoindoline-1,3-dione;
5-(4-((1-(2-(4-(4-chloro-1,2-diphenylbut-1-en-1-yl)phenoxy)ethyl)piperidin-4-yl)methyl)-2,6-dimethylpiperazin-1-yl)-2-(2,6-dioxopiperidin-3-yl)-6-fluoroisoindoline-1,3-dione;
5-(4-((1-(2-(4-(4-chloro-1-(4-hydroxyphenyl)-2-phenylbut-1-en-1-yl)phenoxy)ethyl)piperidin-4-yl)methyl)-2,6-dimethylpiperazin-1-yl)-2-(2,6-dioxopiperidin-3-yl)-6-fluoroisoindoline-1,3-dione;
5-(4-((1-(2-(4-(4-chloro-1,2-bis(4-hydroxyphenyl)but-1-en-1-yl)phenoxy)ethyl)piperidin-4-yl)methyl)-2,6-dimethylpiperazin-1-yl)-2-(2,6-dioxopiperidin-3-yl)-6-fluoroisoindoline-1,3-dione;
5-(4-((1-(2-(4-(4-chloro-1,2-diphenylbut-1-en-1-yl)phenoxy)ethyl)piperidin-4-yl)methyl)-3,5-dimethylpiperazin-1-yl)-2-(2,6-dioxopiperidin-3-yl)isoindoline-1,3-dione;
5-(4-((1-(2-(4-(4-chloro-1-(4-hydroxyphenyl)-2-phenylbut-1-en-1-yl)phenoxy)ethyl)piperidin-4-yl)methyl)-3,5-dimethylpiperazin-1-yl)-2-(2,6-dioxopiperidin-3-yl)isoindoline-1,3-dione;
5-(4-((1-(2-(4-(4-chloro-1,2-bis(4-hydroxyphenyl)but-1-en-1-yl)phenoxy)ethyl)piperidin-4-yl)methyl)-3,5-dimethylpiperazin-1-yl)-2-(2,6-dioxopiperidin-3-yl)isoindoline-1,3-dione;
5-(4-((1-(2-(4-(4-chloro-1,2-diphenylbut-1-en-1-yl)phenoxy)ethyl)piperidin-4-yl)methyl)-3,5-dimethylpiperazin-1-yl)-2-(2,6-dioxopiperidin-3-yl)-6-fluoroisoindoline-1,3-dione;
5-(4-((1-(2-(4-(4-chloro-1-(4-hydroxyphenyl)-2-phenylbut-1-en-1-yl)phenoxy)ethyl)piperidin-4-yl)methyl)-3,5-dimethylpiperazin-1-yl)-2-(2,6-dioxopiperidin-3-yl)-6-fluoroisoindoline-1,3-dione;
5-(4-((1-(2-(4-(4-chloro-1,2-bis(4-hydroxyphenyl)but-1-en-1-yl)phenoxy)ethyl)piperidin-4-yl)methyl)-3,5-dimethylpiperazin-1-yl)-2-(2,6-dioxopiperidin-3-yl)-6-fluoroisoindoline-1,3-dione;
5-(1-((1-(2-(4-(4-chloro-1,2-diphenylbut-1-en-1-yl)phenoxy)ethyl)piperidin-4-yl)methyl)piperidin-4-yl)-2-(2,6-dioxopiperidin-3-yl)isoindoline-1,3-dione;
5-(1-((1-(2-(4-(4-chloro-1-(4-hydroxyphenyl)-2-phenylbut-1-en-1-yl)phenoxy)ethyl)piperidin-4-yl)methyl)piperidin-4-yl)-2-(2,6-dioxopiperidin-3-yl)isoindoline-1,3-dione;
5-(1-((1-(2-(4-(4-chloro-1,2-bis(4-hydroxyphenyl)but-1-en-1-yl)phenoxy)ethyl)piperidin-4-yl)methyl)piperidin-4-yl)-2-(2,6-dioxopiperidin-3-yl)isoindoline-1,3-dione;
5-(1-((1-(2-(4-(4-chloro-1,2-diphenylbut-1-en-1-yl)phenoxy)ethyl)piperidin-4-yl)methyl)piperidin-4-yl)-2-(2,6-dioxopiperidin-3-yl)-6-fluoroisoindoline-1,3-dione;
5-(1-((1-(2-(4-(4-chloro-1-(4-hydroxyphenyl)-2-phenylbut-1-en-1-yl)phenoxy)ethyl)piperidin-4-yl)methyl)piperidin-4-yl)-2-(2,6-dioxopiperidin-3-yl)-6-fluoroisoindoline-1,3-dione;
5-(1-((1-(2-(4-(4-chloro-1,2-bis(4-hydroxyphenyl)but-1-en-1-yl)phenoxy)ethyl)piperidin-4-yl)methyl)piperidin-4-yl)-2-(2,6-dioxopiperidin-3-yl)-6-fluoroisoindoline-1,3-dione;
5-((R)-3-(((1-(2-(4-(4-chloro-1,2-diphenylbut-1-en-1-yl)phenoxy)ethyl)piperidin-4-yl)methyl)amino)piperidin-1-yl)-2-(2,6-dioxopiperidin-3-yl)isoindoline-1,3-dione;
5-((R)-3-(((1-(2-(4-(4-chloro-1-(4-hydroxyphenyl)-2-phenylbut-1-en-1-yl)phenoxy)ethyl)piperidin-4-yl)methyl)amino)piperidin-1-yl)-2-(2,6-dioxopiperidin-3-yl)isoindoline-1,3-dione;
5-((R)-3-(((1-(2-(4-(4-chloro-1,2-bis(4-hydroxyphenyl)but-1-en-1-yl)phenoxy)ethyl)piperidin-4-yl)methyl)amino)piperidin-1-yl)-2-(2,6-dioxopiperidin-3-yl)isoindoline-1,3-dione;
5-((S)-3-(((1-(2-(4-(4-chloro-1,2-diphenylbut-1-en-1-yl)phenoxy)ethyl)piperidin-4-yl)methyl)amino)piperidin-1-yl)-2-(2,6-dioxopiperidin-3-yl)isoindoline-1,3-dione;
5-((S)-3-(((1-(2-(4-(4-chloro-1-(4-hydroxyphenyl)-2-phenylbut-1-en-1-yl)phenoxy)ethyl)piperidin-4-yl)methyl)amino)piperidin-1-yl)-2-(2,6-dioxopiperidin-3-5-((S)-3-(((1-(2-(4-(4-chloro-1,2-bis(4-hydroxyphenyl)but-1-en-1-yl)phenoxy)ethyl)piperidin-4-yl)methyl)amino)piperidin-1-yl)-2-(2,6-dioxopiperidin-3-yl)isoindoline-1,3-dione;
5-(((S)-1-((1-(2-(4-(4-chloro-1,2-diphenylbut-1-en-1-yl)phenoxy)ethyl)piperidin-4-yl)methyl)piperidin-3-yl)amino)-2-(2,6-dioxopiperidin-3-yl)isoindoline-1,3-dione;
5-(((S)-1-((1-(2-(4-(4-chloro-1-(4-hydroxyphenyl)-2-phenylbut-1-en-1-yl)phenoxy)ethyl)piperidin-4-yl)methyl)piperidin-3-yl)amino)-2-(2,6-dioxopiperidin-3-yl)isoindoline-1,3-dione;
5-(((S)-1-((1-(2-(4-(4-chloro-1,2-bis(4-hydroxyphenyl)but-1-en-1-yl)phenoxy)ethyl)piperidin-4-yl)methyl)piperidin-3-yl)amino)-2-(2,6-dioxopiperidin-3-yl)isoindoline-1,3-dione;
5-(((R)-1-((1-(2-(4-(4-chloro-1,2-diphenylbut-1-en-1-yl)phenoxy)ethyl)piperidin-4-yl)methyl)piperidin-3-yl)amino)-2-(2,6-dioxopiperidin-3-yl)isoindoline-1,3-dione;
5-(((R)-1-((1-(2-(4-(4-chloro-1-(4-hydroxyphenyl)-2-phenylbut-1-en-1-yl)phenoxy)ethyl)piperidin-4-yl)methyl)piperidin-3-yl)amino)-2-(2,6-dioxopiperidin-3-yl)isoindoline-1,3-dione;
5-(((R)-1-((1-(2-(4-(4-chloro-1,2-bis(4-hydroxyphenyl)but-1-en-1-yl)phenoxy)ethyl)piperidin-4-yl)methyl)piperidin-3-yl)amino)-2-(2,6-dioxopiperidin-3-yl)isoindoline-1,3-dione;
5-(4-((1-(4-(4-chloro-1-(4-hydroxyphenyl)-2-phenylbut-1-en-1-yl)phenyl)piperidin-4-yl)methyl)piperazin-1-yl)-2-(2,6-dioxopiperidin-3-yl)isoindoline-1,3-dione;
5-(4-((1-(4-(4-chloro-1-(4-hydroxyphenyl)-2-phenylbut-1-en-1-yl)phenyl)piperidin-4-yl)methyl)piperazin-1-yl)-2-(2,6-dioxopiperidin-3-yl)-6-fluoroisoindoline-1,3-dione;
5-(3-((1-(4-(4-chloro-1-(4-hydroxyphenyl)-2-phenylbut-1-en-1-yl)phenyl)piperidin-4-yl)methyl)-3,6-diazabicyclo[3.1.1]heptan-6-yl)-2-(2,6-dioxopiperidin-3-yl)isoindoline-1,3-dione;
5-(3-((1-(4-(4-chloro-1-(4-hydroxyphenyl)-2-phenylbut-1-en-1-yl)phenyl)piperidin-4-yl)methyl)-3,6-diazabicyclo[3.1.1]heptan-6-yl)-2-(2,6-dioxopiperidin-3-yl)-6-fluoroisoindoline-1,3-dione;
5-(6-((1-(4-(4-chloro-1-(4-hydroxyphenyl)-2-phenylbut-1-en-1-yl)phenyl)piperidin-4-yl)methyl)-3,6-diazabicyclo[3.1.1]heptan-3-yl)-2-(2,6-dioxopiperidin-3-yl)isoindoline-1,3-dione;
5-(6-((1-(4-(4-chloro-1-(4-hydroxyphenyl)-2-phenylbut-1-en-1-yl)phenyl)piperidin-4-yl)methyl)-3,6-diazabicyclo[3.1.1]heptan-3-yl)-2-(2,6-dioxopiperidin-3-yl)-6-fluoroisoindoline-1,3-dione;
5-(3-((1-(4-(4-chloro-1-(4-hydroxyphenyl)-2-phenylbut-1-en-1-yl)phenyl)piperidin-4-yl)methyl)-3,8-diazabicyclo[3.2.1]octan-8-yl)-2-(2,6-dioxopiperidin-3-yl)isoindoline-1,3-dione;
5-(3-((1-(4-(4-chloro-1-(4-hydroxyphenyl)-2-phenylbut-1-en-1-yl)phenyl)piperidin-4-yl)methyl)-3,8-diazabicyclo[3.2.1]octan-8-yl)-2-(2,6-dioxopiperidin-3-yl)-6-fluoroisoindoline-1,3-dione;
5-(8-((1-(4-(4-chloro-1-(4-hydroxyphenyl)-2-phenylbut-1-en-1-yl)phenyl)piperidin-4-yl)methyl)-3,8-diazabicyclo[3.2.1]octan-3-yl)-2-(2,6-dioxopiperidin-3-yl)isoindoline-1,3-dione;
5-(8-((1-(4-(4-chloro-1-(4-hydroxyphenyl)-2-phenylbut-1-en-1-yl)phenyl)piperidin-4-yl)methyl)-3,8-diazabicyclo[3.2.1]octan-3-yl)-2-(2,6-dioxopiperidin-3-yl)-6-fluoroisoindoline-1,3-dione;
5-(5-((1-(4-(4-chloro-1-(4-hydroxyphenyl)-2-phenylbut-1-en-1-yl)phenyl)piperidin-4-yl)methyl)-2,5-diazabicyclo[2.2.1]heptan-2-yl)-2-(2,6-dioxopiperidin-3-yl)isoindoline-1,3-dione;
5-(5-((1-(4-(4-chloro-1-(4-hydroxyphenyl)-2-phenylbut-1-en-1-yl)phenyl)piperidin-4-yl)methyl)-2,5-diazabicyclo[2.2.1]heptan-2-yl)-2-(2,6-dioxopiperidin-3-yl)-6-fluoroisoindoline-1,3-dione;
5-(5-((1-(4-(4-chloro-1-(4-hydroxyphenyl)-2-phenylbut-1-en-1-yl)phenyl)piperidin-4-yl)methyl)-2,5-diazabicyclo[2.2.2]octan-2-yl)-2-(2,6-dioxopiperidin-3-yl)isoindoline-1,3-dione;
5-(5-((1-(4-(4-chloro-1-(4-hydroxyphenyl)-2-phenylbut-1-en-1-yl)phenyl)piperidin-4-yl)methyl)-2,5-diazabicyclo[2.2.2]octan-2-yl)-2-(2,6-dioxopiperidin-3-yl)-6-fluoroisoindoline-1,3-dione;
5-(4-((1-(4-(4-chloro-1-(4-hydroxyphenyl)-2-phenylbut-1-en-1-yl)phenyl)piperidin-4-yl)methyl)piperazin-1-yl-2,2,3,3,5,5,6,6-d₈)-2-(2,6-dioxopiperidin-3-yl)isoindoline-1,3-dione;
5-(4-((1-(4-(4-chloro-1-(4-hydroxyphenyl)-2-phenylbut-1-en-1-yl)phenyl)piperidin-4-yl)methyl)piperazin-1-yl-2,2,3,3,5,5,6,6-d₈)-2-(2,6-dioxopiperidin-3-yl)-6-fluoroisoindoline-1,3-dione;
6-(4-((1-(4-(4-chloro-1-(4-hydroxyphenyl)-2-phenylbut-1-en-1-yl)phenyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(4-(4-chloro-1-(4-hydroxyphenyl)-2-phenylbut-1-en-1-yl)phenyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(4-(4-chloro-1-(4-hydroxyphenyl)-2-phenylbut-1-en-1-yl)phenyl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
5-(4-((1-(4-(4-chloro-1-(4-hydroxyphenyl)-2-phenylbut-1-en-1-yl)phenyl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
6-(4-((1-(4-(4-chloro-1-(4-hydroxyphenyl)-2-phenylbut-1-en-1-yl)phenyl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
5-(4-((1-(4-(4-chloro-1-(4-hydroxyphenyl)-2-phenylbut-1-en-1-yl)phenyl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((1-(4-(4-chloro-1-(4-hydroxyphenyl)-2-phenylbut-1-en-1-yl)phenyl)piperidin-4-yl)methyl)-2,6-dimethylpiperazin-1-yl)-2-(2,6-dioxopiperidin-3-yl)isoindoline-1,3-dione;
5-(4-((1-(4-(4-chloro-1-(4-hydroxyphenyl)-2-phenylbut-1-en-1-yl)phenyl)piperidin-4-yl)methyl)-2,6-dimethylpiperazin-1-yl)-2-(2,6-dioxopiperidin-3-yl)-6-fluoroisoindoline-1,3-dione;
5-(4-((1-(4-(4-chloro-1-(4-hydroxyphenyl)-2-phenylbut-1-en-1-yl)phenyl)piperidin-4-yl)methyl)-3,5-dimethylpiperazin-1-yl)-2-(2,6-dioxopiperidin-3-yl)isoindoline-1,3-dione;
5-(4-((1-(4-(4-chloro-1-(4-hydroxyphenyl)-2-phenylbut-1-en-1-yl)phenyl)piperidin-4-yl)methyl)-3,5-dimethylpiperazin-1-yl)-2-(2,6-dioxopiperidin-3-yl)-6-fluoroisoindoline-1,3-dione;
5-(1-((1-(4-(4-chloro-1-(4-hydroxyphenyl)-2-phenylbut-1-en-1-yl)phenyl)piperidin-4-yl)methyl)piperidin-4-yl)-2-(2,6-dioxopiperidin-3-yl)isoindoline-1,3-dione;
5-(1-((1-(4-(4-chloro-1-(4-hydroxyphenyl)-2-phenylbut-1-en-1-yl)phenyl)piperidin-4-yl)methyl)piperidin-4-yl)-2-(2,6-dioxopiperidin-3-yl)-6-fluoroisoindoline-1,3-dione;
5-((R)-3-(((1-(4-(4-chloro-1-(4-hydroxyphenyl)-2-phenylbut-1-en-1-yl)phenyl)piperidin-4-yl)methyl)amino)piperidin-1-yl)-2-(2,6-dioxopiperidin-3-yl)isoindoline-1,3-dione;
5-((S)-3-(((1-(4-(4-chloro-1-(4-hydroxyphenyl)-2-phenylbut-1-en-1-yl)phenyl)piperidin-4-yl)methyl)amino)piperidin-1-yl)-2-(2,6-dioxopiperidin-3-yl)isoindoline-1,3-dione;
5-(((S)-1-((1-(4-(4-chloro-1-(4-hydroxyphenyl)-2-phenylbut-1-en-1-yl)phenyl)piperidin-4-yl)methyl)piperidin-3-yl)amino)-2-(2,6-dioxopiperidin-3-yl)isoindoline-1,3-dione;
5-(((R)-1-((1-(4-(4-chloro-1-(4-hydroxyphenyl)-2-phenylbut-1-en-1-yl)phenyl)piperidin-4-yl)methyl)piperidin-3-yl)amino)-2-(2,6-dioxopiperidin-3-yl)isoindoline-1,3-dione;
5-(4-((1-(4-(4-chloro-1,2-bis(4-hydroxyphenyl)but-1-en-1-yl)phenyl)piperidin-4-yl)methyl)piperazin-1-yl)-2-(2,6-dioxopiperidin-3-yl)isoindoline-1,3-dione;
5-(4-((1-(4-(4-chloro-1,2-bis(4-hydroxyphenyl)but-1-en-1-yl)phenyl)piperidin-4-yl)methyl)piperazin-1-yl)-2-(2,6-dioxopiperidin-3-yl)-6-fluoroisoindoline-1,3-dione;
5-(3-((1-(4-(4-chloro-1,2-bis(4-hydroxyphenyl)but-1-en-1-yl)phenyl)piperidin-4-yl)methyl)-3,6-diazabicyclo[3.1.1]heptan-6-yl)-2-(2,6-dioxopiperidin-3-yl)isoindoline-1,3-dione;
5-(3-((1-(4-(4-chloro-1,2-bis(4-hydroxyphenyl)but-1-en-1-yl)phenyl)piperidin-4-yl)methyl)-3,6-diazabicyclo[3.1.1]heptan-6-yl)-2-(2,6-dioxopiperidin-3-yl)-6-fluoroisoindoline-1,3-dione;
5-(6-((1-(4-(4-chloro-1,2-bis(4-hydroxyphenyl)but-1-en-1-yl)phenyl)piperidin-4-yl)methyl)-3,6-diazabicyclo[3.1.1]heptan-3-yl)-2-(2,6-dioxopiperidin-3-yl)isoindoline-1,3-dione;
5-(6-((1-(4-(4-chloro-1,2-bis(4-hydroxyphenyl)but-1-en-1-yl)phenyl)piperidin-4-yl)methyl)-3,6-diazabicyclo[3.1.1]heptan-3-yl)-2-(2,6-dioxopiperidin-3-yl)-6-fluoroisoindoline-1,3-dione;
5-(3-((1-(4-(4-chloro-1,2-bis(4-hydroxyphenyl)but-1-en-1-yl)phenyl)piperidin-4-yl)methyl)-3,8-diazabicyclo[3.2.1]octan-8-yl)-2-(2,6-dioxopiperidin-3-yl)isoindoline-1,3-dione;
5-(3-((1-(4-(4-chloro-1,2-bis(4-hydroxyphenyl)but-1-en-1-yl)phenyl)piperidin-4-yl)methyl)-3,8-diazabicyclo[3.2.1]octan-8-yl)-2-(2,6-dioxopiperidin-3-yl)-6-fluoroisoindoline-1,3-dione;
5-(8-((1-(4-(4-chloro-1,2-bis(4-hydroxyphenyl)but-1-en-1-yl)phenyl)piperidin-4-yl)methyl)-3,8-diazabicyclo[3.2.1]octan-3-yl)-2-(2,6-dioxopiperidin-3-yl)isoindoline-1,3-dione;
5-(8-((1-(4-(4-chloro-1,2-bis(4-hydroxyphenyl)but-1-en-1-yl)phenyl)piperidin-4-yl)methyl)-3,8-diazabicyclo[3.2.1]octan-3-yl)-2-(2,6-dioxopiperidin-3-yl)-6-fluoroisoindoline-1,3-dione;
5-(5-((1-(4-(4-chloro-1,2-bis(4-hydroxyphenyl)but-1-en-1-yl)phenyl)piperidin-4-yl)methyl)-2,5-diazabicyclo[2.2.1]heptan-2-yl)-2-(2,6-dioxopiperidin-3-yl)isoindoline-1,3-dione;
5-(5-((1-(4-(4-chloro-1,2-bis(4-hydroxyphenyl)but-1-en-1-yl)phenyl)piperidin-4-yl)methyl)-2,5-diazabicyclo[2.2.1]heptan-2-yl)-2-(2,6-dioxopiperidin-3-yl)-6-fluoroisoindoline-1,3-dione;
5-(5-((1-(4-(4-chloro-1,2-bis(4-hydroxyphenyl)but-1-en-1-yl)phenyl)piperidin-4-yl)methyl)-2,5-diazabicyclo[2.2.2]octan-2-yl)-2-(2,6-dioxopiperidin-3-yl)isoindoline-1,3-dione;
5-(5-((1-(4-(4-chloro-1,2-bis(4-hydroxyphenyl)but-1-en-1-yl)phenyl)piperidin-4-yl)methyl)-2,5-diazabicyclo[2.2.2]octan-2-yl)-2-(2,6-dioxopiperidin-3-yl)-6-fluoroisoindoline-1,3-dione;
5-(4-((1-(4-(4-chloro-1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)isoindoline-1,3-dione;
5-(4-((1-(4-(4-chloro-1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-6-fluoroisoindoline-1,3-dione;
6-(4-((1-(4-(4-chloro-1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(4-(4-chloro-1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(4-(4-chloro-1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
5-(4-((1-(4-(4-chloro-1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
6-(4-((1-(4-(4-chloro-1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
5-(4-((1-(4-(4-chloro-1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((1-(4-(4-chloro-1,2-bis(4-hydroxyphenyl)but-1-en-1-yl)phenyl)piperidin-4-yl)methyl)-2,6-dimethylpiperazin-1-yl)-2-(2,6-dioxopiperidin-3-yl)isoindoline-1,3-dione;
5-(4-((1-(4-(4-chloro-1,2-bis(4-hydroxyphenyl)but-1-en-1-yl)phenyl)piperidin-4-yl)methyl)-2,6-dimethylpiperazin-1-yl)-2-(2,6-dioxopiperidin-3-yl)-6-fluoroisoindoline-1,3-dione;
5-(4-((1-(4-(4-chloro-1,2-bis(4-hydroxyphenyl)but-1-en-1-yl)phenyl)piperidin-4-yl)methyl)-3,5-dimethylpiperazin-1-yl)-2-(2,6-dioxopiperidin-3-yl)isoindoline-1,3-dione;
5-(4-((1-(4-(4-chloro-1,2-bis(4-hydroxyphenyl)but-1-en-1-yl)phenyl)piperidin-4-yl)methyl)-3,5-dimethylpiperazin-1-yl)-2-(2,6-dioxopiperidin-3-yl)-6-fluoroisoindoline-1,3-dione;
5-(1-((1-(4-(4-chloro-1,2-bis(4-hydroxyphenyl)but-1-en-1-yl)phenyl)piperidin-4-yl)methyl)piperidin-4-yl)-2-(2,6-dioxopiperidin-3-yl)isoindoline-1,3-dione;
5-(1-((1-(4-(4-chloro-1,2-bis(4-hydroxyphenyl)but-1-en-1-yl)phenyl)piperidin-4-yl)methyl)piperidin-4-yl)-2-(2,6-dioxopiperidin-3-yl)-6-fluoroisoindoline-1,3-dione;
5-((R)-3-(((1-(4-(4-chloro-1,2-bis(4-hydroxyphenyl)but-1-en-1-yl)phenyl)piperidin-4-yl)methyl)amino)piperidin-1-yl)-2-(2,6-dioxopiperidin-3-yl)isoindoline-1,3-dione;
5-((S)-3-(((1-(4-(4-chloro-1,2-bis(4-hydroxyphenyl)but-1-en-1-yl)phenyl)piperidin-4-yl)methyl)amino)piperidin-1-yl)-2-(2,6-dioxopiperidin-3-yl)isoindoline-1,3-dione;
5-(((S)-1-((1-(4-(4-chloro-1,2-bis(4-hydroxyphenyl)but-1-en-1-yl)phenyl)piperidin-4-yl)methyl)piperidin-3-yl)amino)-2-(2,6-dioxopiperidin-3-yl)isoindoline-1,3-dione;
5-(((R)-1-((1-(4-(4-chloro-1,2-bis(4-hydroxyphenyl)but-1-en-1-yl)phenyl)piperidin-4-yl)methyl)piperidin-3-yl)amino)-2-(2,6-dioxopiperidin-3-yl)isoindoline-1,3-dione;
2-(2,6-dioxopiperidin-3-yl)-5-(4-((1-(2-(4-(1,2-diphenylbut-1-en-1-yl)phenoxy)ethyl)piperidin-4-yl)methyl)piperazin-1-yl)isoindoline-1,3-dione;
5-(4-((1-(2-(4-(1,2-bis(4-hydroxyphenyl)but-1-en-1-yl)phenoxy)ethyl)piperidin-4-yl)methyl)piperazin-1-yl)-2-(2,6-dioxopiperidin-3-yl)isoindoline-1,3-dione;
2-(2,6-dioxopiperidin-3-yl)-5-(4-((1-(2-(4-(1,2-diphenylbut-1-en-1-yl)phenoxy)ethyl)piperidin-4-yl)methyl)piperazin-1-yl)-6-fluoroisoindoline-1,3-dione;
2-(2,6-dioxopiperidin-3-yl)-5-fluoro-6-(4-((1-(2-(4-(1-(4-hydroxyphenyl)-2-phenylbut-1-en-1-yl)phenoxy)ethyl)piperidin-4-yl)methyl)piperazin-1-yl)isoindoline-1,3-dione;
2-(2,6-dioxopiperidin-3-yl)-5-fluoro-6-(4-((1-(4-(1-(4-hydroxyphenyl)-2-phenylbut-1-en-1-yl)phenyl)piperidin-4-yl)methyl)piperazin-1-yl)isoindoline-1,3-dione;
5-(4-((1-(2-(4-(1,2-bis(4-hydroxyphenyl)but-1-en-1-yl)phenoxy)ethyl)piperidin-4-yl)methyl)piperazin-1-yl)-2-(2,6-dioxopiperidin-3-yl)-6-fluoroisoindoline-1,3-dione;
2-(2,6-dioxopiperidin-3-yl)-5-(3-((1-(2-(4-(1,2-diphenylbut-1-en-1-yl)phenoxy)ethyl)piperidin-4-yl)methyl)-3,6-diazabicyclo[3.1.1]heptan-6-yl)isoindoline-1,3-dione;
2-(2,6-dioxopiperidin-3-yl)-5-(3-((1-(2-(4-(1-(4-hydroxyphenyl)-2-phenylbut-1-en-1-yl)phenoxy)ethyl)piperidin-4-yl)methyl)-3,6-diazabicyclo[3.1.1]heptan-6-yl)isoindoline-1,3-dione;
5-(3-((1-(2-(4-(1,2-bis(4-hydroxyphenyl)but-1-en-1-yl)phenoxy)ethyl)piperidin-4-yl)methyl)-3,6-diazabicyclo[3.1.1]heptan-6-yl)-2-(2,6-dioxopiperidin-3-yl)isoindoline-1,3-dione;
2-(2,6-dioxopiperidin-3-yl)-5-(3-((1-(2-(4-(1,2-diphenylbut-1-en-1-yl)phenoxy)ethyl)piperidin-4-yl)methyl)-3,6-diazabicyclo[3.1.1]heptan-6-yl)-6-fluoroisoindoline-1,3-dione;
2-(2,6-dioxopiperidin-3-yl)-5-fluoro-6-(3-((1-(2-(4-(1-(4-hydroxyphenyl)-2-phenylbut-1-en-1-yl)phenoxy)ethyl)piperidin-4-yl)methyl)-3,6-diazabicyclo[3.1.1]heptan-6-yl)isoindoline-1,3-dione;
5-(3-((1-(2-(4-(1,2-bis(4-hydroxyphenyl)but-1-en-1-yl)phenoxy)ethyl)piperidin-4-yl)methyl)-3,6-diazabicyclo[3.1.1]heptan-6-yl)-2-(2,6-dioxopiperidin-3-yl)-6-fluoroisoindoline-1,3-dione;
2-(2,6-dioxopiperidin-3-yl)-5-(6-((1-(2-(4-(1,2-diphenylbut-1-en-1-yl)phenoxy)ethyl)piperidin-4-yl)methyl)-3,6-diazabicyclo[3.1.1]heptan-3-yl)isoindoline-1,3-dione;
2-(2,6-dioxopiperidin-3-yl)-5-(6-((1-(2-(4-(1-(4-hydroxyphenyl)-2-phenylbut-1-en-1-yl)phenoxy)ethyl)piperidin-4-yl)methyl)-3,6-diazabicyclo[3.1.1]heptan-3-yl)isoindoline-1,3-dione;
5-(6-((1-(2-(4-(1,2-bis(4-hydroxyphenyl)but-1-en-1-yl)phenoxy)ethyl)piperidin-4-yl)methyl)-3,6-diazabicyclo[3.1.1]heptan-3-yl)-2-(2,6-dioxopiperidin-3-yl)isoindoline-1,3-dione;
2-(2,6-dioxopiperidin-3-yl)-5-(6-((1-(2-(4-(1,2-diphenylbut-1-en-1-yl)phenoxy)ethyl)piperidin-4-yl)methyl)-3,6-diazabicyclo[3.1.1]heptan-3-yl)-6-fluoroisoindoline-1,3-dione;
2-(2,6-dioxopiperidin-3-yl)-5-fluoro-6-(6-((1-(2-(4-(1-(4-hydroxyphenyl)-2-phenylbut-1-en-1-yl)phenoxy)ethyl)piperidin-4-yl)methyl)-3,6-diazabicyclo[3.1.1]heptan-3-yl)isoindoline-1,3-dione;
5-(6-((1-(2-(4-(1,2-bis(4-hydroxyphenyl)but-1-en-1-yl)phenoxy)ethyl)piperidin-4-yl)methyl)-3,6-diazabicyclo[3.1.1]heptan-3-yl)-2-(2,6-dioxopiperidin-3-yl)-6-fluoroisoindoline-1,3-dione;
2-(2,6-dioxopiperidin-3-yl)-5-(3-((1-(2-(4-(1,2-diphenylbut-1-en-1-yl)phenoxy)ethyl)piperidin-4-yl)methyl)-3,8-diazabicyclo[3.2.1]octan-8-yl)isoindoline-1,3-dione;
2-(2,6-dioxopiperidin-3-yl)-5-(3-((1-(2-(4-(1-(4-hydroxyphenyl)-2-phenylbut-1-en-1-yl)phenoxy)ethyl)piperidin-4-yl)methyl)-3,8-diazabicyclo[3.2.1]octan-8-yl)isoindoline-1,3-dione;
5-(3-((1-(2-(4-(1,2-bis(4-hydroxyphenyl)but-1-en-1-yl)phenoxy)ethyl)piperidin-4-yl)methyl)-3,8-diazabicyclo[3.2.1]octan-8-yl)-2-(2,6-dioxopiperidin-3-yl)isoindoline-1,3-dione;
2-(2,6-dioxopiperidin-3-yl)-5-(3-((1-(2-(4-(1,2-diphenylbut-1-en-1-yl)phenoxy)ethyl)piperidin-4-yl)methyl)-3,8-diazabicyclo[3.2.1]octan-8-yl)-6-fluoroisoindoline-1,3-dione;
2-(2,6-dioxopiperidin-3-yl)-5-fluoro-6-(3-((1-(2-(4-(1-(4-hydroxyphenyl)-2-phenylbut-1-en-1-yl)phenoxy)ethyl)piperidin-4-yl)methyl)-3,8-diazabicyclo[3.2.1]octan-8-yl)isoindoline-1,3-dione;
5-(3-((1-(2-(4-(1,2-bis(4-hydroxyphenyl)but-1-en-1-yl)phenoxy)ethyl)piperidin-4-yl)methyl)-3,8-diazabicyclo[3.2.1]octan-8-yl)-2-(2,6-dioxopiperidin-3-yl)-6-fluoroisoindoline-1,3-dione;
2-(2,6-dioxopiperidin-3-yl)-5-(8-((1-(2-(4-(1,2-diphenylbut-1-en-1-yl)phenoxy)ethyl)piperidin-4-yl)methyl)-3,8-diazabicyclo[3.2.1]octan-3-yl)isoindoline-1,3-dione;
2-(2,6-dioxopiperidin-3-yl)-5-(8-((1-(2-(4-(1-(4-hydroxyphenyl)-2-phenylbut-1-en-1-yl)phenoxy)ethyl)piperidin-4-yl)methyl)-3,8-diazabicyclo[3.2.1]octan-3-yl)isoindoline-1,3-dione;
5-(8-((1-(2-(4-(1,2-bis(4-hydroxyphenyl)but-1-en-1-yl)phenoxy)ethyl)piperidin-4-yl)methyl)-3,8-diazabicyclo[3.2.1]octan-3-yl)-2-(2,6-dioxopiperidin-3-yl)isoindoline-1,3-dione;
2-(2,6-dioxopiperidin-3-yl)-5-(8-((1-(2-(4-(1,2-diphenylbut-1-en-1-yl)phenoxy)ethyl)piperidin-4-yl)methyl)-3,8-diazabicyclo[3.2.1]octan-3-yl)-6-fluoroisoindoline-1,3-dione;
2-(2,6-dioxopiperidin-3-yl)-5-fluoro-6-(8-((1-(2-(4-(1-(4-hydroxyphenyl)-2-phenylbut-1-en-1-yl)phenoxy)ethyl)piperidin-4-yl)methyl)-3,8-diazabicyclo[3.2.1]octan-3-yl)isoindoline-1,3-dione;
5-(8-((1-(2-(4-(1,2-bis(4-hydroxyphenyl)but-1-en-1-yl)phenoxy)ethyl)piperidin-4-yl)methyl)-3,8-diazabicyclo[3.2.1]octan-3-yl)-2-(2,6-dioxopiperidin-3-yl)-6-fluoroisoindoline-1,3-dione;
2-(2,6-dioxopiperidin-3-yl)-5-(5-((1-(2-(4-(1,2-diphenylbut-1-en-1-yl)phenoxy)ethyl)piperidin-4-yl)methyl)-2,5-diazabicyclo[2.2.1]heptan-2-yl)isoindoline-1,3-dione;
5-(5-((1-(2-(4-(1,2-bis(4-hydroxyphenyl)but-1-en-1-yl)phenoxy)ethyl)piperidin-4-yl)methyl)-2,5-diazabicyclo[2.2.1]heptan-2-yl)-2-(2,6-dioxopiperidin-3-yl)isoindoline-1,3-dione;
2-(2,6-dioxopiperidin-3-yl)-5-(5-((1-(2-(4-(1,2-diphenylbut-1-en-1-yl)phenoxy)ethyl)piperidin-4-yl)methyl)-2,5-diazabicyclo[2.2.1]heptan-2-yl)-6-fluoroisoindoline-1,3-dione;
2-(2,6-dioxopiperidin-3-yl)-5-fluoro-6-(5-((1-(2-(4-(1-(4-hydroxyphenyl)-2-phenylbut-1-en-1-yl)phenoxy)ethyl)piperidin-4-yl)methyl)-2,5-diazabicyclo[2.2.1]heptan-2-yl)isoindoline-1,3-dione;
5-(5-((1-(2-(4-(1,2-bis(4-hydroxyphenyl)but-1-en-1-yl)phenoxy)ethyl)piperidin-4-yl)methyl)-2,5-diazabicyclo[2.2.1]heptan-2-yl)-2-(2,6-dioxopiperidin-3-yl)-6-fluoroisoindoline-1,3-dione;
2-(2,6-dioxopiperidin-3-yl)-5-(5-((1-(2-(4-(1,2-diphenylbut-1-en-1-yl)phenoxy)ethyl)piperidin-4-yl)methyl)-2,5-diazabicyclo[2.2.2]octan-2-yl)isoindoline-1,3-dione;
2-(2,6-dioxopiperidin-3-yl)-5-(5-((1-(2-(4-(1-(4-hydroxyphenyl)-2-phenylbut-1-en-1-yl)phenoxy)ethyl)piperidin-4-yl)methyl)-2,5-diazabicyclo[2.2.2]octan-2-yl)isoindoline-1,3-dione;
5-(5-((1-(2-(4-(1,2-bis(4-hydroxyphenyl)but-1-en-1-yl)phenoxy)ethyl)piperidin-4-yl)methyl)-2,5-diazabicyclo[2.2.2]octan-2-yl)-2-(2,6-dioxopiperidin-3-yl)isoindoline-1,3-dione;
2-(2,6-dioxopiperidin-3-yl)-5-(5-((1-(2-(4-(1,2-diphenylbut-1-en-1-yl)phenoxy)ethyl)piperidin-4-yl)methyl)-2,5-diazabicyclo[2.2.2]octan-2-yl)-6-fluoroisoindoline-1,3-dione;
2-(2,6-dioxopiperidin-3-yl)-5-fluoro-6-(5-((1-(2-(4-(1-(4-hydroxyphenyl)-2-phenylbut-1-en-1-yl)phenoxy)ethyl)piperidin-4-yl)methyl)-2,5-diazabicyclo[2.2.2]octan-2-yl)isoindoline-1,3-dione;
5-(5-((1-(2-(4-(1,2-bis(4-hydroxyphenyl)but-1-en-1-yl)phenoxy)ethyl)piperidin-4-yl)methyl)-2,5-diazabicyclo[2.2.2]octan-2-yl)-2-(2,6-dioxopiperidin-3-yl)-6-fluoroisoindoline-1,3-dione;
2-(2,6-dioxopiperidin-3-yl)-5-(4-((1-(2-(4-(1,2-diphenylbut-1-en-1-yl)phenoxy)ethyl)piperidin-4-yl)methyl)piperazin-1-yl-2,2,3,3,5,5,6,6-d₈)isoindoline-1,3-dione;
2-(2,6-dioxopiperidin-3-yl)-5-(4-((1-(2-(4-(1-(4-hydroxyphenyl)-2-phenylbut-1-en-1-yl)phenoxy)ethyl)piperidin-4-yl)methyl)piperazin-1-yl-2,2,3,3,5,5,6,6-d₈)isoindoline-1,3-dione;
5-(4-((1-(2-(4-(1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)isoindoline-1,3-dione;
2-(2,6-dioxopiperidin-3-yl)-5-(4-((1-(2-(4-(1,2-diphenylbut-1-en-1-yl)phenoxy)ethyl)piperidin-4-yl)methyl)piperazin-1-yl-2,2,3,3,5,5,6,6-d₈)-6-fluoroisoindoline-1,3-dione;
2-(2,6-dioxopiperidin-3-yl)-5-fluoro-6-(4-((1-(2-(4-(1-(4-hydroxyphenyl)-2-phenylbut-1-en-1-yl)phenoxy)ethyl)piperidin-4-yl)methyl)piperazin-1-yl-2,2,3,3,5,5,6,6-d₈)isoindoline-1,3-dione;
5-(4-((1-(2-(4-(1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-6-fluoroisoindoline-1,3-dione;
2-(2,6-dioxopiperidin-3-yl)-6-(4-((1-(2-(4-(1,2-diphenylbut-1-en-1-yl)phenoxy)ethyl)piperidin-4-yl)methyl)piperazin-1-yl-2,2,3,3,5,5,6,6-d₈)-4-fluoroisoindoline-1,3-dione;
2-(2,6-dioxopiperidin-3-yl)-4-fluoro-6-(4-((1-(2-(4-(1-(4-hydroxyphenyl)-2-phenylbut-1-en-1-yl)phenoxy)ethyl)piperidin-4-yl)methyl)piperazin-1-yl-2,2,3,3,5,5,6,6-d₈)isoindoline-1,3-dione;
6-(4-((1-(2-(4-(1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
2-(2,6-dioxopiperidin-3-yl)-5-(4-((1-(2-(4-(1,2-diphenylbut-1-en-1-yl)phenoxy)ethyl)piperidin-4-yl)methyl)piperazin-1-yl-2,2,3,3,5,5,6,6-d₈)-4-fluoroisoindoline-1,3-dione;
2-(2,6-dioxopiperidin-3-yl)-4-fluoro-5-(4-((1-(2-(4-(1-(4-hydroxyphenyl)-2-phenylbut-1-en-1-yl)phenoxy)ethyl)piperidin-4-yl)methyl)piperazin-1-yl-2,2,3,3,5,5,6,6-d₈)isoindoline-1,3-dione;
5-(4-((1-(2-(4-(1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
2-(2,6-dioxopiperidin-3-yl)-5-(4-((1-(2-(4-(1,2-diphenylbut-1-en-1-yl)phenoxy)ethyl)piperidin-4-yl)methyl)piperazin-1-yl-2,2,3,3,5,5,6,6-d₈)-4,6-difluoroisoindoline-1,3-dione;
2-(2,6-dioxopiperidin-3-yl)-4,6-difluoro-5-(4-((1-(2-(4-(1-(4-hydroxyphenyl)-2-phenylbut-1-en-1-yl)phenoxy)ethyl)piperidin-4-yl)methyl)piperazin-1-yl-2,2,3,3,5,5,6,6-d₈)isoindoline-1,3-dione;
5-(4-((1-(2-(4-(1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
2-(2,6-dioxopiperidin-3-yl)-5-(4-((1-(2-(4-(1,2-diphenylbut-1-en-1-yl)phenoxy)ethyl)piperidin-4-yl)methyl)piperazin-1-yl-2,2,3,3,5,5,6,6-d₈)-4,7-difluoroisoindoline-1,3-dione;
2-(2,6-dioxopiperidin-3-yl)-4,7-difluoro-5-(4-((1-(2-(4-(1-(4-hydroxyphenyl)-2-phenylbut-1-en-1-yl)phenoxy)ethyl)piperidin-4-yl)methyl)piperazin-1-yl-2,2,3,3,5,5,6,6-d₈)isoindoline-1,3-dione;
5-(4-((1-(2-(4-(1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
2-(2,6-dioxopiperidin-3-yl)-6-(4-((1-(2-(4-(1,2-diphenylbut-1-en-1-yl)phenoxy)ethyl)piperidin-4-yl)methyl)piperazin-1-yl-2,2,3,3,5,5,6,6-d₈)-4,5-difluoroisoindoline-1,3-dione;
2-(2,6-dioxopiperidin-3-yl)-4,5-difluoro-6-(4-((1-(2-(4-(1-(4-hydroxyphenyl)-2-phenylbut-1-en-1-yl)phenoxy)ethyl)piperidin-4-yl)methyl)piperazin-1-yl-2,2,3,3,5,5,6,6-d₈)isoindoline-1,3-dione;
6-(4-((1-(2-(4-(1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
2-(2,6-dioxopiperidin-3-yl)-5-(4-((1-(2-(4-(1,2-diphenylbut-1-en-1-yl)phenoxy)ethyl)piperidin-4-yl)methyl)piperazin-1-yl-2,2,3,3,5,5,6,6-d₈)-4,6,7-trifluoroisoindoline-1,3-dione;
2-(2,6-dioxopiperidin-3-yl)-4,5,7-trifluoro-6-(4-((1-(2-(4-(1-(4-hydroxyphenyl)-2-phenylbut-1-en-1-yl)phenoxy)ethyl)piperidin-4-yl)methyl)piperazin-1-yl-2,2,3,3,5,5,6,6-d₈)isoindoline-1,3-dione;
5-(4-((1-(2-(4-(1,2-bis(4-hydroxyphenyl)but-1-en-1-yl)phenoxy)ethyl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
2-(2,6-dioxopiperidin-3-yl)-5-(4-((1-(2-(4-(1,2-diphenylbut-1-en-1-yl)phenoxy)ethyl)piperidin-4-yl)methyl)-2,6-dimethylpiperazin-1-yl)isoindoline-1,3-dione;
2-(2,6-dioxopiperidin-3-yl)-5-(4-((1-(2-(4-(1-(4-hydroxyphenyl)-2-phenylbut-1-en-1-yl)phenoxy)ethyl)piperidin-4-yl)methyl)-2,6-dimethylpiperazin-1-yl)isoindoline-1,3-dione;
5-(4-((1-(2-(4-(1,2-bis(4-hydroxyphenyl)but-1-en-1-yl)phenoxy)ethyl)piperidin-4-yl)methyl)-2,6-dimethylpiperazin-1-yl)-2-(2,6-dioxopiperidin-3-yl)isoindoline-1,3-dione;
2-(2,6-dioxopiperidin-3-yl)-5-(4-((1-(2-(4-(1,2-diphenylbut-1-en-1-yl)phenoxy)ethyl)piperidin-4-yl)methyl)-2,6-dimethylpiperazin-1-yl)-6-fluoroisoindoline-1,3-dione;
2-(2,6-dioxopiperidin-3-yl)-5-fluoro-6-(4-((1-(2-(4-(1-(4-hydroxyphenyl)-2-phenylbut-1-en-1-yl)phenoxy)ethyl)piperidin-4-yl)methyl)-2,6-dimethylpiperazin-1-yl)isoindoline-1,3-dione;
5-(4-((1-(2-(4-(1,2-bis(4-hydroxyphenyl)but-1-en-1-yl)phenoxy)ethyl)piperidin-4-yl)methyl)-2,6-dimethylpiperazin-1-yl)-2-(2,6-dioxopiperidin-3-yl)-6-fluoroisoindoline-1,3-dione;
2-(2,6-dioxopiperidin-3-yl)-5-(4-((1-(2-(4-(1,2-diphenylbut-1-en-1-yl)phenoxy)ethyl)piperidin-4-yl)methyl)-3,5-dimethylpiperazin-1-yl)isoindoline-1,3-dione;
2-(2,6-dioxopiperidin-3-yl)-5-(4-((1-(2-(4-(1-(4-hydroxyphenyl)-2-phenylbut-1-en-1-yl)phenoxy)ethyl)piperidin-4-yl)methyl)-3,5-dimethylpiperazin-1-yl)isoindoline-1,3-dione;
5-(4-((1-(2-(4-(1,2-bis(4-hydroxyphenyl)but-1-en-1-yl)phenoxy)ethyl)piperidin-4-yl)methyl)-3,5-dimethylpiperazin-1-yl)-2-(2,6-dioxopiperidin-3-yl)isoindoline-1,3-dione;
2-(2,6-dioxopiperidin-3-yl)-5-(4-((1-(2-(4-(1,2-diphenylbut-1-en-1-yl)phenoxy)ethyl)piperidin-4-yl)methyl)-3,5-dimethylpiperazin-1-yl)-6-fluoroisoindoline-1,3-dione;
2-(2,6-dioxopiperidin-3-yl)-5-fluoro-6-(4-((1-(2-(4-(1-(4-hydroxyphenyl)-2-phenylbut-1-en-1-yl)phenoxy)ethyl)piperidin-4-yl)methyl)-3,5-dimethylpiperazin-1-yl)isoindoline-1,3-dione;
5-(4-((1-(2-(4-(1,2-bis(4-hydroxyphenyl)but-1-en-1-yl)phenoxy)ethyl)piperidin-4-yl)methyl)-3,5-dimethylpiperazin-1-yl)-2-(2,6-dioxopiperidin-3-yl)-6-fluoroisoindoline-1,3-dione;
2-(2,6-dioxopiperidin-3-yl)-5-(1-((1-(2-(4-(1,2-diphenylbut-1-en-1-yl)phenoxy)ethyl)piperidin-4-yl)methyl)piperidin-4-yl)isoindoline-1,3-dione;
2-(2,6-dioxopiperidin-3-yl)-5-(1-((1-(2-(4-(1-(4-hydroxyphenyl)-2-phenylbut-1-en-1-yl)phenoxy)ethyl)piperidin-4-yl)methyl)piperidin-4-yl)isoindoline-1,3-dione;
5-(1-((1-(2-(4-(1,2-bis(4-hydroxyphenyl)but-1-en-1-yl)phenoxy)ethyl)piperidin-4-yl)methyl)piperidin-4-yl)-2-(2,6-dioxopiperidin-3-yl)isoindoline-1,3-dione;
2-(2,6-dioxopiperidin-3-yl)-5-(1-((1-(2-(4-(1,2-diphenylbut-1-en-1-yl)phenoxy)ethyl)piperidin-4-yl)methyl)piperidin-4-yl)-6-fluoroisoindoline-1,3-dione;
2-(2,6-dioxopiperidin-3-yl)-5-fluoro-6-(1-((1-(2-(4-(1-(4-hydroxyphenyl)-2-phenylbut-1-en-1-yl)phenoxy)ethyl)piperidin-4-yl)methyl)piperidin-4-yl)isoindoline-1,3-dione;
5-(1-((1-(2-(4-(1,2-bis(4-hydroxyphenyl)but-1-en-1-yl)phenoxy)ethyl)piperidin-4-yl)methyl)piperidin-4-yl)-2-(2,6-dioxopiperidin-3-yl)-6-fluoroisoindoline-1,3-dione;
2-(2,6-dioxopiperidin-3-yl)-5-((R)-3-(((1-(2-(4-(1,2-diphenylbut-1-en-1-yl)phenoxy)ethyl)piperidin-4-yl)methyl)amino)piperidin-1-yl)isoindoline-1,3-dione;
2-(2,6-dioxopiperidin-3-yl)-5-((R)-3-(((1-(2-(4-(1-(4-hydroxyphenyl)-2-phenylbut-1-en-1-yl)phenoxy)ethyl)piperidin-4-yl)methyl)amino)piperidin-1-yl)isoindoline-1,3-dione;
5-((R)-3-(((1-(2-(4-(1,2-bis(4-hydroxyphenyl)but-1-en-1-yl)phenoxy)ethyl)piperidin-4-yl)methyl)amino)piperidin-1-yl)-2-(2,6-dioxopiperidin-3-yl)isoindoline-1,3-dione;
2-(2,6-dioxopiperidin-3-yl)-5-((S)-3-(((1-(2-(4-(1,2-diphenylbut-1-en-1-yl)phenoxy)ethyl)piperidin-4-yl)methyl)amino)piperidin-1-yl)isoindoline-1,3-dione;
2-(2,6-dioxopiperidin-3-yl)-5-((S)-3-(((1-(2-(4-(1-(4-hydroxyphenyl)-2-phenylbut-1-en-1-yl)phenoxy)ethyl)piperidin-4-yl)methyl)amino)piperidin-1-yl)isoindoline-1,3-dione;
5-((S)-3-(((1-(2-(4-(1,2-bis(4-hydroxyphenyl)but-1-en-1-yl)phenoxy)ethyl)piperidin-4-yl)methyl)amino)piperidin-1-yl)-2-(2,6-dioxopiperidin-3-yl)isoindoline-1,3-dione;
2-(2,6-dioxopiperidin-3-yl)-5-(((S)-1-((1-(2-(4-(1,2-diphenylbut-1-en-1-yl)phenoxy)ethyl)piperidin-4-yl)methyl)piperidin-3-yl)amino)isoindoline-1,3-dione;
2-(2,6-dioxopiperidin-3-yl)-5-(((S)-1-((1-(2-(4-(1-(4-hydroxyphenyl)-2-phenylbut-1-en-1-yl)phenoxy)ethyl)piperidin-4-yl)methyl)piperidin-3-yl)amino)isoindoline-1,3-dione;
5-(((S)-1-((1-(2-(4-(1,2-bis(4-hydroxyphenyl)but-1-en-1-yl)phenoxy)ethyl)piperidin-4-yl)methyl)piperidin-3-yl)amino)-2-(2,6-dioxopiperidin-3-yl)isoindoline-1,3-dione;
2-(2,6-dioxopiperidin-3-yl)-5-(((R)-1-((1-(2-(4-(1,2-diphenylbut-1-en-1-yl)phenoxy)ethyl)piperidin-4-yl)methyl)piperidin-3-yl)amino)isoindoline-1,3-dione;
2-(2,6-dioxopiperidin-3-yl)-5-(((R)-1-((1-(2-(4-(1-(4-hydroxyphenyl)-2-phenylbut-1-en-1-yl)phenoxy)ethyl)piperidin-4-yl)methyl)piperidin-3-yl)amino)isoindoline-1,3-dione;
5-(((R)-1-((1-(2-(4-(1,2-bis(4-hydroxyphenyl)but-1-en-1-yl)phenoxy)ethyl)piperidin-4-yl)methyl)piperidin-3-yl)amino)-2-(2,6-dioxopiperidin-3-yl)isoindoline-1,3-dione;
2-(2,6-dioxopiperidin-3-yl)-5-(4-((1-(4-(1-(4-hydroxyphenyl)-2-phenylbut-1-en-1-yl)phenyl)piperidin-4-yl)methyl)piperazin-1-yl)isoindoline-1,3-dione;
5-(4-((1-(4-(4-chloro-1-(4-hydroxyphenyl)-2-phenylbut-1-en-1-yl)phenyl)piperidin-4-yl)methyl)piperazin-1-yl)-2-(2,6-dioxopiperidin-3-yl)-6-fluoroisoindoline-1,3-dione;
2-(2,6-dioxopiperidin-3-yl)-5-(3-((1-(4-(1-(4-hydroxyphenyl)-2-phenylbut-1-en-1-yl)phenyl)piperidin-4-yl)methyl)-3,6-diazabicyclo[3.1.1]heptan-6-yl)isoindoline-1,3-dione;
2-(2,6-dioxopiperidin-3-yl)-5-fluoro-6-(3-((1-(4-(1-(4-hydroxyphenyl)-2-phenylbut-1-en-1-yl)phenyl)piperidin-4-yl)methyl)-3,6-diazabicyclo[3.1.1]heptan-6-yl)isoindoline-1,3-dione;
2-(2,6-dioxopiperidin-3-yl)-5-(6-((1-(4-(1-(4-hydroxyphenyl)-2-phenylbut-1-en-1-yl)phenyl)piperidin-4-yl)methyl)-3,6-diazabicyclo[3.1.1]heptan-3-yl)isoindoline-1,3-dione;
2-(2,6-dioxopiperidin-3-yl)-5-fluoro-6-(6-((1-(4-(1-(4-hydroxyphenyl)-2-phenylbut-1-en-1-yl)phenyl)piperidin-4-yl)methyl)-3,6-diazabicyclo[3.1.1]heptan-3-yl)isoindoline-1,3-dione;
2-(2,6-dioxopiperidin-3-yl)-5-(3-((1-(4-(1-(4-hydroxyphenyl)-2-phenylbut-1-en-1-yl)phenyl)piperidin-4-yl)methyl)-3,8-diazabicyclo[3.2.1]octan-8-yl)isoindoline-1,3-dione;
2-(2,6-dioxopiperidin-3-yl)-5-fluoro-6-(3-((1-(4-(1-(4-hydroxyphenyl)-2-phenylbut-1-en-1-yl)phenyl)piperidin-4-yl)methyl)-3,8-diazabicyclo[3.2.1]octan-8-yl)isoindoline-1,3-dione;
2-(2,6-dioxopiperidin-3-yl)-5-(8-((1-(4-(1-(4-hydroxyphenyl)-2-phenylbut-1-en-1-yl)phenyl)piperidin-4-yl)methyl)-3,8-diazabicyclo[3.2.1]octan-3-yl)isoindoline-1,3-dione;
2-(2,6-dioxopiperidin-3-yl)-5-fluoro-6-(8-((1-(4-(1-(4-hydroxyphenyl)-2-phenylbut-1-en-1-yl)phenyl)piperidin-4-yl)methyl)-3,8-diazabicyclo[3.2.1]octan-3-yl)isoindoline-1,3-dione;
2-(2,6-dioxopiperidin-3-yl)-5-(5-((1-(4-(1-(4-hydroxyphenyl)-2-phenylbut-1-en-1-yl)phenyl)piperidin-4-yl)methyl)-2,5-diazabicyclo[2.2.1]heptan-2-yl)isoindoline-1,3-dione;
2-(2,6-dioxopiperidin-3-yl)-5-fluoro-6-(5-((1-(4-(1-(4-hydroxyphenyl)-2-phenylbut-1-en-1-yl)phenyl)piperidin-4-yl)methyl)-2,5-diazabicyclo[2.2.1]heptan-2-yl)isoindoline-1,3-dione;
2-(2,6-dioxopiperidin-3-yl)-5-(5-((1-(4-(1-(4-hydroxyphenyl)-2-phenylbut-1-en-1-yl)phenyl)piperidin-4-yl)methyl)-2,5-diazabicyclo[2.2.2]octan-2-yl)isoindoline-1,3-dione;
2-(2,6-dioxopiperidin-3-yl)-5-fluoro-6-(5-((1-(4-(1-(4-hydroxyphenyl)-2-phenylbut-1-en-1-yl)phenyl)piperidin-4-yl)methyl)-2,5-diazabicyclo[2.2.2]octan-2-yl)isoindoline-1,3-dione;
2-(2,6-dioxopiperidin-3-yl)-5-(4-((1-(4-(1-(4-hydroxyphenyl)-2-phenylbut-1-en-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-5-fluoro-6-(4-((1-(4-(1-(4-hydroxyphenyl)-2-phenylbut-1-en-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-4-fluoro-6-(4-((1-(4-(1-(4-hydroxyphenyl)-2-phenylbut-1-en-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-4-fluoro-5-(4-((1-(4-(1-(4-hydroxyphenyl)-2-phenylbut-1-en-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-4,6-difluoro-5-(4-((1-(4-(1-(4-hydroxyphenyl)-2-phenylbut-1-en-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-4,7-difluoro-5-(4-((1-(4-(1-(4-hydroxyphenyl)-2-phenylbut-1-en-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-4,5-difluoro-6-(4-((1-(4-(1-(4-hydroxyphenyl)-2-phenylbut-1-en-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-4,5,7-trifluoro-6-(4-((1-(4-(1-(4-hydroxyphenyl)-2-phenylbut-1-en-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-5-(4-((1-(4-(1-(4-hydroxyphenyl)-2-phenylbut-1-en-1-yl)phenyl)piperidin-4-yl)methyl)-2,6-dimethylpiperazin-1-yl)isoindoline-1,3-dione;
2-(2,6-dioxopiperidin-3-yl)-5-fluoro-6-(4-((1-(4-(1-(4-hydroxyphenyl)-2-phenylbut-1-en-1-yl)phenyl)piperidin-4-yl)methyl)-2,6-dimethylpiperazin-1-yl)isoindoline-1,3-dione;
2-(2,6-dioxopiperidin-3-yl)-5-(4-((1-(4-(1-(4-hydroxyphenyl)-2-phenylbut-1-en-1-yl)phenyl)piperidin-4-yl)methyl)-3,5-dimethylpiperazin-1-yl)isoindoline-1,3-dione;
2-(2,6-dioxopiperidin-3-yl)-5-fluoro-6-(4-((1-(4-(1-(4-hydroxyphenyl)-2-phenylbut-1-en-1-yl)phenyl)piperidin-4-yl)methyl)-3,5-dimethylpiperazin-1-yl)isoindoline-1,3-dione;
2-(2,6-dioxopiperidin-3-yl)-5-(1-((1-(4-(1-(4-hydroxyphenyl)-2-phenylbut-1-en-1-yl)phenyl)piperidin-4-yl)methyl)piperidin-4-yl)isoindoline-1,3-dione;
2-(2,6-dioxopiperidin-3-yl)-5-fluoro-6-(1-((1-(4-(1-(4-hydroxyphenyl)-2-phenylbut-1-en-1-yl)phenyl)piperidin-4-yl)methyl)piperidin-4-yl)isoindoline-1,3-dione;
2-(2,6-dioxopiperidin-3-yl)-5-((R)-3-(((1-(4-(1-(4-hydroxyphenyl)-2-phenylbut-1-en-1-yl)phenyl)piperidin-4-yl)methyl)amino)piperidin-1-yl)isoindoline-1,3-dione;
2-(2,6-dioxopiperidin-3-yl)-5-((S)-3-(((1-(4-(1-(4-hydroxyphenyl)-2-phenylbut-1-en-1-yl)phenyl)piperidin-4-yl)methyl)amino)piperidin-1-yl)isoindoline-1,3-dione;
2-(2,6-dioxopiperidin-3-yl)-5-(((S)-1-((1-(4-(1-(4-hydroxyphenyl)-2-phenylbut-1-en-1-yl)phenyl)piperidin-4-yl)methyl)piperidin-3-yl)amino)isoindoline-1,3-dione;
2-(2,6-dioxopiperidin-3-yl)-5-(((R)-1-((1-(4-(1-(4-hydroxyphenyl)-2-phenylbut-1-en-1-yl)phenyl)piperidin-4-yl)methyl)piperidin-3-yl)amino)isoindoline-1,3-dione;
5-(4-((1-(4-(1,2-bis(4-hydroxyphenyl)but-1-en-1-yl)phenyl)piperidin-4-yl)methyl)piperazin-1-yl)-2-(2,6-dioxopiperidin-3-yl)isoindoline-1,3-dione;
5-(4-((1-(4-(1,2-bis(4-hydroxyphenyl)but-1-en-1-yl)phenyl)piperidin-4-yl)methyl)piperazin-1-yl)-2-(2,6-dioxopiperidin-3-yl)-6-fluoroisoindoline-1,3-dione;
5-(3-((1-(4-(1,2-bis(4-hydroxyphenyl)but-1-en-1-yl)phenyl)piperidin-4-yl)methyl)-3,6-diazabicyclo[3.1.1]heptan-6-yl)-2-(2,6-dioxopiperidin-3-yl)isoindoline-1,3-dione;
5-(3-((1-(4-(1,2-bis(4-hydroxyphenyl)but-1-en-1-yl)phenyl)piperidin-4-yl)methyl)-3,6-diazabicyclo[3.1.1]heptan-6-yl)-2-(2,6-dioxopiperidin-3-yl)-6-fluoroisoindoline-1,3-dione;
5-(6-((1-(4-(1,2-bis(4-hydroxyphenyl)but-1-en-1-yl)phenyl)piperidin-4-yl)methyl)-3,6-diazabicyclo[3.1.1]heptan-3-yl)-2-(2,6-dioxopiperidin-3-yl)isoindoline-1,3-dione;
5-(6-((1-(4-(1,2-bis(4-hydroxyphenyl)but-1-en-1-yl)phenyl)piperidin-4-yl)methyl)-3,6-diazabicyclo[3.1.1]heptan-3-yl)-2-(2,6-dioxopiperidin-3-yl)-6-fluoroisoindoline-1,3-dione;
5-(3-((1-(4-(1,2-bis(4-hydroxyphenyl)but-1-en-1-yl)phenyl)piperidin-4-yl)methyl)-3,8-diazabicyclo[3.2.1]octan-8-yl)-2-(2,6-dioxopiperidin-3-yl)isoindoline-1,3-dione;
5-(3-((1-(4-(1,2-bis(4-hydroxyphenyl)but-1-en-1-yl)phenyl)piperidin-4-yl)methyl)-3,8-diazabicyclo[3.2.1]octan-8-yl)-2-(2,6-dioxopiperidin-3-yl)-6-fluoroisoindoline-1,3-dione;
5-(8-((1-(4-(1,2-bis(4-hydroxyphenyl)but-1-en-1-yl)phenyl)piperidin-4-yl)methyl)-3,8-diazabicyclo[3.2.1]octan-3-yl)-2-(2,6-dioxopiperidin-3-yl)isoindoline-1,3-dione;
5-(8-((1-(4-(1,2-bis(4-hydroxyphenyl)but-1-en-1-yl)phenyl)piperidin-4-yl)methyl)-3,8-diazabicyclo[3.2.1]octan-3-yl)-2-(2,6-dioxopiperidin-3-yl)-6-fluoroisoindoline-1,3-dione;
5-(5-((1-(4-(1,2-bis(4-hydroxyphenyl)but-1-en-1-yl)phenyl)piperidin-4-yl)methyl)-2,5-diazabicyclo[2.2.1]heptan-2-yl)-2-(2,6-dioxopiperidin-3-yl)isoindoline-1,3-dione;
5-(5-((1-(4-(1,2-bis(4-hydroxyphenyl)but-1-en-1-yl)phenyl)piperidin-4-yl)methyl)-2,5-diazabicyclo[2.2.1]heptan-2-yl)-2-(2,6-dioxopiperidin-3-yl)-6-fluoroisoindoline-1,3-dione;
5-(5-((1-(4-(1,2-bis(4-hydroxyphenyl)but-1-en-1-yl)phenyl)piperidin-4-yl)methyl)-2,5-diazabicyclo[2.2.2]octan-2-yl)-2-(2,6-dioxopiperidin-3-yl)isoindoline-1,3-dione;
5-(5-((1-(4-(1,2-bis(4-hydroxyphenyl)but-1-en-1-yl)phenyl)piperidin-4-yl)methyl)-2,5-diazabicyclo[2.2.2]octan-2-yl)-2-(2,6-dioxopiperidin-3-yl)-6-fluoroisoindoline-1,3-dione;
5-(4-((1-(4-(1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)isoindoline-1,3-dione;
5-(4-((1-(4-(1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-6-fluoroisoindoline-1,3-dione;
6-(4-((1-(4-(1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(4-(1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-4-fluoroisoindoline-1,3-dione;
5-(4-((1-(4-(1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-4,6-difluoroisoindoline-1,3-dione;
5-(4-((1-(4-(1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-4,7-difluoroisoindoline-1,3-dione;
6-(4-((1-(4-(1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-4,5-difluoroisoindoline-1,3-dione;
5-(4-((1-(4-(1,2-bis(4-hydroxyphenyl)but-1-en-1-yl)phenyl)piperidin-4-yl)methyl)piperazin-1-yl-2,2,3,3,5,5,6,6-d₈)-2-(2,6-dioxopiperidin-3-yl)-4,6,7-trifluoroisoindoline-1,3-dione;
5-(4-((1-(4-(1,2-bis(4-hydroxyphenyl)but-1-en-1-yl)phenyl)piperidin-4-yl)methyl)-2,6-dimethylpiperazin-1-yl)-2-(2,6-dioxopiperidin-3-yl)isoindoline-1,3-dione;
5-(4-((1-(4-(1,2-bis(4-hydroxyphenyl)but-1-en-1-yl)phenyl)piperidin-4-yl)methyl)-2,6-dimethylpiperazin-1-yl)-2-(2,6-dioxopiperidin-3-yl)-6-fluoroisoindoline-1,3-dione;
5-(4-((1-(4-(1,2-bis(4-hydroxyphenyl)but-1-en-1-yl)phenyl)piperidin-4-yl)methyl)-3,5-dimethylpiperazin-1-yl)-2-(2,6-dioxopiperidin-3-yl)isoindoline-1,3-dione;
5-(4-((1-(4-(1,2-bis(4-hydroxyphenyl)but-1-en-1-yl)phenyl)piperidin-4-yl)methyl)-3,5-dimethylpiperazin-1-yl)-2-(2,6-dioxopiperidin-3-yl)-6-fluoroisoindoline-1,3-dione;
5-(1-((1-(4-(1,2-bis(4-hydroxyphenyl)but-1-en-1-yl)phenyl)piperidin-4-yl)methyl)piperidin-4-yl)-2-(2,6-dioxopiperidin-3-yl)isoindoline-1,3-dione;
5-(1-((1-(4-(1,2-bis(4-hydroxyphenyl)but-1-en-1-yl)phenyl)piperidin-4-yl)methyl)piperidin-4-yl)-2-(2,6-dioxopiperidin-3-yl)-6-fluoroisoindoline-1,3-dione;
5-((R)-3-(((1-(4-(1,2-bis(4-hydroxyphenyl)but-1-en-1-yl)phenyl)piperidin-4-yl)methyl)amino)piperidin-1-yl)-2-(2,6-dioxopiperidin-3-yl)isoindoline-1,3-dione;
5-((S)-3-(((1-(4-(1,2-bis(4-hydroxyphenyl)but-1-en-1-yl)phenyl)piperidin-4-yl)methyl)amino)piperidin-1-yl)-2-(2,6-dioxopiperidin-3-yl)isoindoline-1,3-dione;
5-(((S)-1-((1-(4-(1,2-bis(4-hydroxyphenyl)but-1-en-1-yl)phenyl)piperidin-4-yl)methyl)piperidin-3-yl)amino)-2-(2,6-dioxopiperidin-3-yl)isoindoline-1,3-dione;
5-(((R)-1-((1-(4-(1,2-bis(4-hydroxyphenyl)but-1-en-1-yl)phenyl)piperidin-4-yl)methyl)piperidin-3-yl)amino)-2-(2,6-dioxopiperidin-3-yl)isoindoline-1,3-dione;
2-(2,6-dioxopiperidin-3-yl)-5-fluoro-6-(4-((1-(4-((1R,2S)-6-hydroxy-2-phenyl-1,2,3,4-tetrahydronaphthalen-1-yl)phenyl)piperidin-4-yl)methyl)piperazin-1-yl)isoindoline-1,3-dione;
2-(2,6-dioxopiperidin-3-yl)-5-(3-((1-(4-((1R,2S)-6-hydroxy-2-phenyl-1,2,3,4-tetrahydronaphthalen-1-yl)phenyl)piperidin-4-yl)methyl)-3,6-diazabicyclo[3.1.1]heptan-6-yl)isoindoline-1,3-dione;
2-(2,6-dioxopiperidin-3-yl)-5-fluoro-6-(3-((1-(4-((1R,2S)-6-hydroxy-2-phenyl-1,2,3,4-tetrahydronaphthalen-1-yl)phenyl)piperidin-4-yl)methyl)-3,6-diazabicyclo[3.1.1]heptan-6-yl)isoindoline-1,3-dione;
2-(2,6-dioxopiperidin-3-yl)-5-(6-((1-(4-((1R,2S)-6-hydroxy-2-phenyl-1,2,3,4-tetrahydronaphthalen-1-yl)phenyl)piperidin-4-yl)methyl)-3,6-diazabicyclo[3.1.1]heptan-3-yl)isoindoline-1,3-dione;
2-(2,6-dioxopiperidin-3-yl)-5-fluoro-6-(6-((1-(4-((1R,2S)-6-hydroxy-2-phenyl-1,2,3,4-tetrahydronaphthalen-1-yl)phenyl)piperidin-4-yl)methyl)-3,6-diazabicyclo[3.1.1]heptan-3-yl)isoindoline-1,3-dione;
2-(2,6-dioxopiperidin-3-yl)-5-(3-((1-(4-((1R,2S)-6-hydroxy-2-phenyl-1,2,3,4-tetrahydronaphthalen-1-yl)phenyl)piperidin-4-yl)methyl)-3,8-diazabicyclo[3.2.1]octan-8-2-(2,6-dioxopiperidin-3-yl)-5-fluoro-6-(3-((1-(4-((1R,2S)-6-hydroxy-2-phenyl-1,2,3,4-tetrahydronaphthalen-1-yl)phenyl)piperidin-4-yl)methyl)-3,8-diazabicyclo[3.2.1]octan-8-yl)isoindoline-1,3-dione;
2-(2,6-dioxopiperidin-3-yl)-5-(8-((1-(4-((1R,2S)-6-hydroxy-2-phenyl-1,2,3,4-tetrahydronaphthalen-1-yl)phenyl)piperidin-4-yl)methyl)-3,8-diazabicyclo[3.2.1]octan-3-yl)isoindoline-1,3-dione;
2-(2,6-dioxopiperidin-3-yl)-5-fluoro-6-(8-((1-(4-((1R,2S)-6-hydroxy-2-phenyl-1,2,3,4-tetrahydronaphthalen-1-yl)phenyl)piperidin-4-yl)methyl)-3,8-diazabicyclo[3.2.1]octan-3-yl)isoindoline-1,3-dione;
2-(2,6-dioxopiperidin-3-yl)-5-fluoro-6-(5-((1-(4-((1R,2S)-6-hydroxy-2-phenyl-1,2,3,4-tetrahydronaphthalen-1-yl)phenyl)piperidin-4-yl)methyl)-2,5-diazabicyclo[2.2.1]heptan-2-yl)isoindoline-1,3-dione;
2-(2,6-dioxopiperidin-3-yl)-5-(5-((1-(4-((1R,2S)-6-hydroxy-2-phenyl-1,2,3,4-tetrahydronaphthalen-1-yl)phenyl)piperidin-4-yl)methyl)-2,5-diazabicyclo[2.2.2]octan-2-yl)isoindoline-1,3-dione;
2-(2,6-dioxopiperidin-3-yl)-5-fluoro-6-(5-((1-(4-((1R,2S)-6-hydroxy-2-phenyl-1,2,3,4-tetrahydronaphthalen-1-yl)phenyl)piperidin-4-yl)methyl)-2,5-diazabicyclo[2.2.2]octan-2-yl)isoindoline-1,3-dione;
2-(2,6-dioxopiperidin-3-yl)-5-(4-((1-(4-((1R,2S)-6-hydroxy-2-phenyl-1,2,3,4-tetrahydronaphthalen-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-5-fluoro-6-(4-((1-(4-((1R,2S)-6-hydroxy-2-phenyl-1,2,3,4-tetrahydronaphthalen-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-4-fluoro-6-(4-((1-(4-((1R,2S)-6-hydroxy-2-phenyl-1,2,3,4-tetrahydronaphthalen-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-4-fluoro-5-(4-((1-(4-((1R,2S)-6-hydroxy-2-phenyl-1,2,3,4-tetrahydronaphthalen-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-4,6-difluoro-5-(4-((1-(4-((1R,2S)-6-hydroxy-2-phenyl-1,2,3,4-tetrahydronaphthalen-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-4,7-difluoro-5-(4-((1-(4-((1R,2S)-6-hydroxy-2-phenyl-1,2,3,4-tetrahydronaphthalen-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-4,5-difluoro-6-(4-((1-(4-((1R,2S)-6-hydroxy-2-phenyl-1,2,3,4-tetrahydronaphthalen-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-4,5,7-trifluoro-6-(4-((1-(4-((1R,2S)-6-hydroxy-2-phenyl-1,2,3,4-tetrahydronaphthalen-1-yl)phenyl)piperidin-4-yl)methyl)piperazin-1-yl-2,2,3,3,5,5,6,6-d₈)isoindoline-1,3-dione;
2-(2,6-dioxopiperidin-3-yl)-5-(4-((1-(4-((1R,2S)-6-hydroxy-2-phenyl-1,2,3,4-tetrahydronaphthalen-1-yl)phenyl)piperidin-4-yl)methyl)-2,6-dimethylpiperazin-1-yl)isoindoline-1,3-dione;
2-(2,6-dioxopiperidin-3-yl)-5-fluoro-6-(4-((1-(4-((1R,2S)-6-hydroxy-2-phenyl-1,2,3,4-tetrahydronaphthalen-1-yl)phenyl)piperidin-4-yl)methyl)-2,6-dimethylpiperazin-1-yl)isoindoline-1,3-dione;
2-(2,6-dioxopiperidin-3-yl)-5-(4-((1-(4-((1R,2S)-6-hydroxy-2-phenyl-1,2,3,4-tetrahydronaphthalen-1-yl)phenyl)piperidin-4-yl)methyl)-3,5-dimethylpiperazin-1-yl)isoindoline-1,3-dione;
2-(2,6-dioxopiperidin-3-yl)-5-fluoro-6-(4-((1-(4-((1R,2S)-6-hydroxy-2-phenyl-1,2,3,4-tetrahydronaphthalen-1-yl)phenyl)piperidin-4-yl)methyl)-3,5-dimethylpiperazin-1-yl)isoindoline-1,3-dione;
2-(2,6-dioxopiperidin-3-yl)-5-(1-((1-(4-((1R,2S)-6-hydroxy-2-phenyl-1,2,3,4-tetrahydronaphthalen-1-yl)phenyl)piperidin-4-yl)methyl)piperidin-4-yl)isoindoline-1,3-dione;
2-(2,6-dioxopiperidin-3-yl)-5-fluoro-6-(1-((1-(4-((1R,2S)-6-hydroxy-2-phenyl-1,2,3,4-tetrahydronaphthalen-1-yl)phenyl)piperidin-4-yl)methyl)piperidin-4-yl)isoindoline-1,3-dione;
2-(2,6-dioxopiperidin-3-yl)-5-((R)-3-(((1-(4-((1R,2S)-6-hydroxy-2-phenyl-1,2,3,4-tetrahydronaphthalen-1-yl)phenyl)piperidin-4-yl)methyl)amino)piperidin-1-yl)isoindoline-1,3-dione;
2-(2,6-dioxopiperidin-3-yl)-5-((S)-3-(((1-(4-((1R,2S)-6-hydroxy-2-phenyl-1,2,3,4-tetrahydronaphthalen-1-yl)phenyl)piperidin-4-yl)methyl)amino)piperidin-1-yl)isoindoline-1,3-dione;
2-(2,6-dioxopiperidin-3-yl)-5-(((S)-1-((1-(4-((1R,2S)-6-hydroxy-2-phenyl-1,2,3,4-tetrahydronaphthalen-1-yl)phenyl)piperidin-4-yl)methyl)piperidin-3-yl)amino)isoindoline-1,3-dione;
2-(2,6-dioxopiperidin-3-yl)-5-(((R)-1-((1-(4-((1R,2S)-6-hydroxy-2-phenyl-1,2,3,4-tetrahydronaphthalen-1-yl)phenyl)piperidin-4-yl)methyl)piperidin-3-yl)amino)isoindoline-1,3-dione;
2-(2,6-dioxopiperidin-3-yl)-5-fluoro-6-(4-((1-(2-(4-((1R,2S)-6-hydroxy-2-phenyl-1,2,3,4-tetrahydronaphthalen-1-yl)phenoxy)ethyl)piperidin-4-yl)methyl)piperazin-1-yl)isoindoline-1,3-dione;
2-(2,6-dioxopiperidin-3-yl)-5-(3-((1-(2-(4-((1R,2S)-6-hydroxy-2-phenyl-1,2,3,4-tetrahydronaphthalen-1-yl)phenoxy)ethyl)piperidin-4-yl)methyl)-3,6-diazabicyclo[3.1.1]heptan-6-yl)isoindoline-1,3-dione;
2-(2,6-dioxopiperidin-3-yl)-5-fluoro-6-(3-((1-(2-(4-((1R,2S)-6-hydroxy-2-phenyl-1,2,3,4-tetrahydronaphthalen-1-yl)phenoxy)ethyl)piperidin-4-yl)methyl)-3,6-diazabicyclo[3.1.1]heptan-6-yl)isoindoline-1,3-dione;
2-(2,6-dioxopiperidin-3-yl)-5-(6-((1-(2-(4-((1R,2S)-6-hydroxy-2-phenyl-1,2,3,4-tetrahydronaphthalen-1-yl)phenoxy)ethyl)piperidin-4-yl)methyl)-3,6-diazabicyclo[3.1.1]heptan-3-yl)isoindoline-1,3-dione;
2-(2,6-dioxopiperidin-3-yl)-5-fluoro-6-(6-((1-(2-(4-((1R,2S)-6-hydroxy-2-phenyl-1,2,3,4-tetrahydronaphthalen-1-yl)phenoxy)ethyl)piperidin-4-yl)methyl)-3,6-diazabicyclo[3.1.1]heptan-3-yl)isoindoline-1,3-dione;
2-(2,6-dioxopiperidin-3-yl)-5-(3-((1-(2-(4-((1R,2S)-6-hydroxy-2-phenyl-1,2,3,4-tetrahydronaphthalen-1-yl)phenoxy)ethyl)piperidin-4-yl)methyl)-3,8-diazabicyclo[3.2.1]octan-8-yl)isoindoline-1,3-dione;
2-(2,6-dioxopiperidin-3-yl)-5-fluoro-6-(3-((1-(2-(4-((1R,2S)-6-hydroxy-2-phenyl-1,2,3,4-tetrahydronaphthalen-1-yl)phenoxy)ethyl)piperidin-4-yl)methyl)-3,8-diazabicyclo[3.2.1]octan-8-yl)isoindoline-1,3-dione;
2-(2,6-dioxopiperidin-3-yl)-5-(8-((1-(2-(4-((1R,2S)-6-hydroxy-2-phenyl-1,2,3,4-tetrahydronaphthalen-1-yl)phenoxy)ethyl)piperidin-4-yl)methyl)-3,8-diazabicyclo[3.2.1]octan-3-yl)isoindoline-1,3-dione;
2-(2,6-dioxopiperidin-3-yl)-5-fluoro-6-(8-((1-(2-(4-((1R,2S)-6-hydroxy-2-phenyl-1,2,3,4-tetrahydronaphthalen-1-yl)phenoxy)ethyl)piperidin-4-yl)methyl)-3,8-diazabicyclo[3.2.1]octan-3-2-(2,6-dioxopiperidin-3-yl)-5-fluoro-6-(5-((1-(2-(4-((1R,2S)-6-hydroxy-2-phenyl-1,2,3,4-tetrahydronaphthalen-1-yl)phenoxy)ethyl)piperidin-4-yl)methyl)-2,5-diazabicyclo[2.2.1]heptan-2-yl)isoindoline-1,3-dione;
2-(2,6-dioxopiperidin-3-yl)-5-fluoro-6-(5-((1-(2-(4-((1R,2S)-6-hydroxy-2-phenyl-1,2,3,4-tetrahydronaphthalen-1-yl)phenoxy)ethyl)piperidin-4-yl)methyl)-2,5-diazabicyclo[2.2.2]octan-2-yl)isoindoline-1,3-dione;
2-(2,6-dioxopiperidin-3-yl)-5-(4-((1-(2-(4-((1R,2S)-6-hydroxy-2-phenyl-1,2,3,4-tetrahydronaphthalen-1-yl)phenoxy)ethyl)piperidin-4-yl)methyl)piperazin-1-yl-2,2,3,3,5,5,6,6-d₈)isoindoline-1,3-dione;
2-(2,6-dioxopiperidin-3-yl)-5-fluoro-6-(4-((1-(2-(4-((1R,2S)-6-hydroxy-2-phenyl-1,2,3,4-tetrahydronaphthalen-1-yl)phenoxy)ethyl)piperidin-4-yl)methyl)piperazin-1-yl-2,2,3,3,5,5,6,6-d₈)isoindoline-1,3-dione;
2-(2,6-dioxopiperidin-3-yl)-4-fluoro-6-(4-((1-(2-(4-((1R,2S)-6-hydroxy-2-phenyl-1,2,3,4-tetrahydronaphthalen-1-yl)phenoxy)ethyl)piperidin-4-yl)methyl)piperazin-1-yl-2,2,3,3,5,5,6,6-d₈)isoindoline-1,3-dione;
2-(2,6-dioxopiperidin-3-yl)-4-fluoro-5-(4-((1-(2-(4-((1R,2S)-6-hydroxy-2-phenyl-1,2,3,4-tetrahydronaphthalen-1-yl)phenoxy)ethyl)piperidin-4-yl)methyl)piperazin-1-yl-2,2,3,3,5,5,6,6-d₈)isoindoline-1,3-dione;
2-(2,6-dioxopiperidin-3-yl)-4,6-difluoro-5-(4-((1-(2-(4-((1R,2S)-6-hydroxy-2-phenyl-1,2,3,4-tetrahydronaphthalen-1-yl)phenoxy)ethyl)piperidin-4-yl)methyl)piperazin-1-yl-2,2,3,3,5,5,6,6-d₈)isoindoline-1,3-dione;
2-(2,6-dioxopiperidin-3-yl)-4,7-difluoro-5-(4-((1-(2-(4-((1R,2S)-6-hydroxy-2-phenyl-1,2,3,4-tetrahydronaphthalen-1-yl)phenoxy)ethyl)piperidin-4-yl)methyl)piperazin-1-yl-2,2,3,3,5,5,6,6-d₈)isoindoline-1,3-dione;
2-(2,6-dioxopiperidin-3-yl)-4,5-difluoro-6-(4-((1-(2-(4-((1R,2S)-6-hydroxy-2-phenyl-1,2,3,4-tetrahydronaphthalen-1-yl)phenoxy)ethyl)piperidin-4-yl)methyl)piperazin-1-yl-2,2,3,3,5,5,6,6-d₈)isoindoline-1,3-dione;
2-(2,6-dioxopiperidin-3-yl)-4,5,7-trifluoro-6-(4-((1-(2-(4-((1R,2S)-6-hydroxy-2-phenyl-1,2,3,4-tetrahydronaphthalen-1-yl)phenoxy)ethyl)piperidin-4-yl)methyl)piperazin-1-yl-2,2,3,3,5,5,6,6-d₈)isoindoline-1,3-dione;
2-(2,6-dioxopiperidin-3-yl)-5-(4-((1-(2-(4-((1R,2S)-6-hydroxy-2-phenyl-1,2,3,4-tetrahydronaphthalen-1-yl)phenoxy)ethyl)piperidin-4-yl)methyl)-2,6-dimethylpiperazin-1-yl)isoindoline-1,3-dione;
2-(2,6-dioxopiperidin-3-yl)-5-fluoro-6-(4-((1-(2-(4-((1R,2S)-6-hydroxy-2-phenyl-1,2,3,4-tetrahydronaphthalen-1-yl)phenoxy)ethyl)piperidin-4-yl)methyl)-2,6-dimethylpiperazin-1-yl)isoindoline-1,3-dione;
2-(2,6-dioxopiperidin-3-yl)-5-(4-((1-(2-(4-((1R,2S)-6-hydroxy-2-phenyl-1,2,3,4-tetrahydronaphthalen-1-yl)phenoxy)ethyl)piperidin-4-yl)methyl)-3,5-dimethylpiperazin-1-yl)isoindoline-1,3-dione;
2-(2,6-dioxopiperidin-3-yl)-5-fluoro-6-(4-((1-(2-(4-((1R,2S)-6-hydroxy-2-phenyl-1,2,3,4-tetrahydronaphthalen-1-yl)phenoxy)ethyl)piperidin-4-yl)methyl)-3,5-dimethylpiperazin-1-yl)isoindoline-1,3-dione;
2-(2,6-dioxopiperidin-3-yl)-5-(1-((1-(2-(4-((1R,2S)-6-hydroxy-2-phenyl-1,2,3,4-tetrahydronaphthalen-1-yl)phenoxy)ethyl)piperidin-4-yl)methyl)piperidin-4-yl)isoindoline-1,3-dione;
2-(2,6-dioxopiperidin-3-yl)-5-fluoro-6-(1-((1-(2-(4-((1R,2S)-6-hydroxy-2-phenyl-1,2,3,4-tetrahydronaphthalen-1-yl)phenoxy)ethyl)piperidin-4-yl)methyl)piperidin-4-yl)isoindoline-1,3-dione;
2-(2,6-dioxopiperidin-3-yl)-5-((R)-3-(((1-(2-(4-((1R,2S)-6-hydroxy-2-phenyl-1,2,3,4-tetrahydronaphthalen-1-yl)phenoxy)ethyl)piperidin-4-yl)methyl)amino)piperidin-1-yl)isoindoline-1,3-dione;
2-(2,6-dioxopiperidin-3-yl)-5-((S)-3-(((1-(2-(4-((1R,2S)-6-hydroxy-2-phenyl-1,2,3,4-tetrahydronaphthalen-1-yl)phenoxy)ethyl)piperidin-4-yl)methyl)amino)piperidin-1-yl)isoindoline-1,3-dione;
2-(2,6-dioxopiperidin-3-yl)-5-(((S)-1-((1-(2-(4-((1R,2S)-6-hydroxy-2-phenyl-1,2,3,4-tetrahydronaphthalen-1-yl)phenoxy)ethyl)piperidin-4-yl)methyl)piperidin-3-yl)amino)isoindoline-1,3-dione;
2-(2,6-dioxopiperidin-3-yl)-5-(((R)-1-((1-(2-(4-((1R,2S)-6-hydroxy-2-phenyl-1,2,3,4-tetrahydronaphthalen-1-yl)phenoxy)ethyl)piperidin-4-yl)methyl)piperidin-3-yl)amino)isoindoline-1,3-dione;
N-((1r,4r)-4-(3-chloro-4-cyanophenoxy)cyclohexyl)-6-(4-((8-(2-(2,6-dioxopiperidin-3-yl)-1-oxoisoindolin-5-yl)-3,8-diazabicyclo[3.2.1]octan-3-yl)methyl)piperidin-1-yl)pyridazine-3-carboxamide;
N-((1r,4r)-4-(3-chloro-4-cyanophenoxy)cyclohexyl)-6-(4-((3-(2-(2,6-dioxopiperidin-3-yl)-1-oxoisoindolin-5-yl)-3,8-diazabicyclo[3.2.1]octan-8-yl)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-1-oxoisoindolin-5-yl)piperidin-1-yl)methyl)piperidin-1-yl)pyridazine-3-carboxamide;
N-((1r,4r)-4-(3-chloro-4-cyanophenoxy)cyclohexyl)-6-(4-(((3S,4S)-4-(2-(2,6-dioxopiperidin-3-yl)-1-oxoisoindolin-5-yl)-3,4-dihydroxypiperidin-1-yl)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-1-oxoisoindolin-5-yl)-3,6-dihydropyridin-1(2H)-yl)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-4-fluoro-1-oxoisoindolin-5-yl)piperidin-1-yl)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-4-fluoro-1-oxoisoindolin-5-yl)-3,6-dihydropyridin-1(2H)-yl)methyl)piperidin-1-yl)pyridazine-3-carboxamide;
N-((1r,4r)-4-(3-chloro-4-cyanophenoxy)cyclohexyl)-6-(4-((4-(2-(2,6-dioxopiperidin-3-yl)-7-fluoro-1-oxoisoindolin-5-yl)piperidin-1-yl)methyl)piperidin-1-yl)pyridazine-3-carboxamide;
N-((1r,3r)-3-(3-chloro-4-cyanophenoxy)-2,2,4,4-tetramethylcyclobutyl)-6-(4-((4-(2-(2,6-dioxopiperidin-3-yl)-1-oxoisoindolin-5-yl)piperidin-1-yl)methyl)piperidin-1-yl)nicotinamide;
N-((1r,3r)-3-(3-chloro-4-cyanophenoxy)-2,2,4,4-tetramethylcyclobutyl)-6-(4-((4-(2-(2,6-dioxopiperidin-3-yl)-1-oxoisoindolin-5-yl)-3,6-dihydropyridin-1(2H)-yl)methyl)piperidin-1-yl)nicotinamide;
7-cyclopentyl-2-((5-(4-((4-((2-(2,6-dioxopiperidin-3-yl)-1-oxoisoindolin-5-yl)amino)piperidin-1-yl)methyl)piperidin-1-yl)pyridin-2-yl)amino)-N,N-dimethyl-7H-pyrrolo[2,3-d]pyrimidine-6-carboxamide;
N-(2-chloro-6-methylphenyl)-2-((6-(4-((4-(2-(2,6-dioxopiperidin-3-yl)-1-oxoisoindolin-5-yl)piperidin-1-yl)methyl)piperidin-1-yl)-2-methylpyrimidin-4-yl)amino)thiazole-5-carboxamide; and
N-(2-chloro-6-methylphenyl)-2-((6-(4-(((3S,4S)-4-(2-(2,6-dioxopiperidin-3-yl)-1-oxoisoindolin-5-yl)-3,4-dihydroxypiperidin-1-yl)methyl)piperidin-1-yl)-2-methylpyrimidin-4-yl)amino)thiazole-5-carboxamide.

38. The compound of Formula (III) or salts, enantiomers, stereoisomers, solvates, isotopically enriched analogs, prodrugs, or polymorphs thereof as claimed in claim 20, which is hydrohalides (including hydrochlorides and hydrobromates), sulfates, maleates, sulfonates, citrates, lactates, lactobionates, L-tartrates, fumarates, L-malates, L-lactates, α-Ketoglutarates, hippurates, D-glucuronates, D-gluconates, α-D-glucoheptonates, glycolates, mucates, L-ascorbates, orotates, picrates, glycinates, alaninates, argininates, cinnamates, laurates, pamoates, sebacates, benzenesulfonates, methanesulfonates, ethanesulfonates, edisylates, formates, acetates, 2,2-dichloroacetates, trimethylacetates, propionates, valerates, palmitates, triphenylacetates, 2-ethylsuccinates, iodates, niacinates, L-pyroglutamates, L-prolinates, ferulates, 2-hydroxyethanesulfonates, nitrates, gentisates, cholates, salicylates, terephthalates, glutarates, adipates, stearates, oleates, undecenoates, camphorates, camphorsulfonates, dodecyl sulfonates, phosphates, thiocyanates, dihydrophosphates, pyrophosphates, metaphosphates, oxalates, carbonates, malonates, benzoates, mandelates, succinates, trifluoroacetates, pyruvates, para-chlorobenzenesulfonates, 1,5-naphthalenedisulfonates, 3-hydroxy-2-naphthoates, 1-hydroxy-2-naphthoates, 2-naphthalenesulfonates, hydroxyacetates, or p-toluenesulfonates of the compound of Formula (III).

39. A pharmaceutical composition comprising as active ingredient the compound of Formula (I) or pharmaceutically acceptable salts, enantiomers, stereoisomers, solvates, isotopically enriched analogs, prodrugs, or polymorphs thereof as claimed in claim 1 and at least one pharmaceutically acceptable carrier;
optionally comprising at least one additional therapeutic agent.

40. (canceled)

41. A pharmaceutical composition comprising as active ingredient the compound of Formula (III) or pharmaceutically acceptable salts, enantiomers, stereoisomers, solvates, isotopically enriched analogs, prodrugs, or polymorphs thereof as claimed in claim 20, and at least one pharmaceutically acceptable carrier;
optionally comprising at least one additional therapeutic agent, e.g., an anticancer agent.

42. (canceled)

43. A medicine kit or reagent kit comprising:
the compound of Formula (I) or a pharmaceutically acceptable salt thereof as claimed in claim 1, or a pharmaceutical composition comprising the compound of Formula (I) or a pharmaceutically acceptable salt thereof.

44. A method, comprising preparing of the compound of Formula (III) as claimed in claim 20 from a compound of Formula (I): 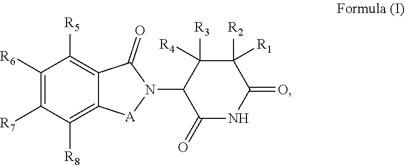
wherein A represents CO, CH₂ or CD₂;
R₁, R₂, R₃ and R₄ are the same or different and each independently represent hydrogen, deuterium, halogen, optionally deuterated C₁₋₆ alkyl, optionally deuterated C₁₋₆ alkoxy, or halogenated C₁₋₆ alkyl;
R₅, R₆, R₇ and R₈ are the same or different and each independently represent R₉, hydrogen, deuterium, halogen, hydroxy, mercapto, nitro, amino, cyano, optionally deuterated C₁₋₆ alkyl, optionally deuterated C₁₋₆ alkoxy, halogenated C₁₋₆ alkyl, C₂₋₆ alkenyl, or C₂₋₆ alkynyl, wherein one to three of R₅, R₆, R₇ and R₈ represents R₉;
R₉ represents R—U— group, wherein
U represents a bond, or U represents N(R₁₀) or O, wherein R₁₀ represents H or optionally deuterated C₁₋₆ alkyl;
R represents optionally substituted nitrogen-containing heterocyclyl, said optionally substituted nitrogen-containing heterocyclyl is optionally substituted nitrogen-containing monocyclic heterocyclyl or optionally substituted nitrogen-containing bridged heterocyclyl;
wherein
when two to four of R₅, R₆, R₇, and R₈ independently represent R₉, the compound of Formula (I) is optionally partially or fully deuterated; or
when only one of R₅, R₆, R₇, and R₈ represents R₉, the compound of Formula (I) is optionally partially or fully deuterated;
wherein in case that only one of R₅, R₆, R₇, and R₈ represents R₉ and the compound of Formula (I) is not deuterated:
when R represents optionally substituted nitrogen-containing bridged heterocyclyl, the following compounds are excluded:
5-(2,5-diazabicyclo[2.2.1]heptan-2-yl)-2-(2,6-dioxopyridin-3-yl)isoindoline-1,3-dione;
5-(2,5-diazabicyclo[2.2.1]heptan-2-yl)-2-(2,6-dioxopyridin-3-yl)-6-fluoroisoindoline-1,3-dione;
5-(2,5-diazabicyclo[2.2.1]heptan-2-yl)-2-(2,6-dioxopyridin-3-yl)-6-fluoroisoindoline-1,3-dione;
3-(5-(2,5-diazabicyclo[2.2.1]heptan-2-yl)-1-oxoisoindolin-2-yl)piperidine-2,6-dione;
3-(5-(8-azabicyclo[3.2.1]octan-3-yl)-1-oxoisoindolin-2-yl)piperidine-2,6-dione; and
3-(5-(9-ethyl-3-methyl-3,9-diazabicyclo[3.3.1]nonan-7-yl)-1-oxoisoindolin-2-yl)piperidine-2,6-dione; or
when A represents CO and one of R₆ and R₇ represents R₉, where R represents substituted nitrogen-containing monocyclic heterocyclyl, and the other one of R₆ and R₇, as well as R₅ and R₈ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, one to three of the other one of R₆ and R₇ as well as R₅ and R₈ represents halogen, and the nitrogen-containing monocyclic heterocyclyl is substituted with one or more substituents selected from the group consisting of hydroxy, amino, mercapto, nitro, halogen, cyano, C₁₋₆ alkyl, C₁₋₆ alkoxy, halogenated C₁₋₆ alkyl, NH₂—C₁₋₆ alkylene, C₁₋₆ alkyl-NH—, C₁₋₆ alkyl-NHC(O)—, C₁₋₆ alkyl-C(O)NH— or any combination thereof;
when A represents CO and one of R₆ and R₇ represents R₉, where R represents unsubstituted nitrogen-containing monocyclic heterocyclyl, the other one of R₆ and R₇ represents hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, and R₅ and R₈ each independently represent halogen;
when A represents CO and one of R₅ and R₈ represents R₉, where R represents substituted nitrogen-containing monocyclic heterocyclyl, and the other one of R₅ and R₈, as well as R₆ and R₇ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, one to three of the other one of R₅ and R₈ as well as R₆ and R₇ represents halogen, and the nitrogen-containing monocyclic heterocyclyl is substituted with one or more substituents selected from the group consisting of hydroxy, amino, mercapto, nitro, halogen, cyano, CHO, C₁₋₆ alkyl, halogenated C₁₋₆ alkyl, C₃₋₆ cycloalkyl, C₁₋₆ alkoxy, C₁₋₆ alkyl-NH—, NH₂—C₁₋₆ alkylene, C₁₋₆ alkyl-NHC(O)—, C₁₋₆ alkyl-C(O)NH— or any combination thereof;
when A represents CO and one of R₅ and R₈ represents R₉, where R represents unsubstituted nitrogen-containing monocyclic heterocyclyl, the other one of R₅ and R₈ and/or R₇ each independently represent halogen, and R₆ represents hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl;
when A represents CH₂ and one of R₆ and R₇ represents R₉, where R represents substituted nitrogen-containing monocyclic heterocyclyl, and the other one of R₆ and R₇, as well as R₅ and R₈ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, one to three of the other one of R₆ and R₇ as well as R₅ and R₈ represents halogen, and the nitrogen-containing monocyclic heterocyclyl is substituted with one or more substituents selected from the group consisting of hydroxy, amino, mercapto, nitro, halogen, CHO, cyano, C₁₋₆ alkyl, halogenated C₁₋₆ alkyl, C₃₋₆ cycloalkyl, C₁₋₆ alkoxy, C₁₋₆ alkyl-NH—, NH₂—C₁₋₆ alkylene, C₁₋₆ alkyl-NHC(O)—, C₁₋₆ alkyl-C(O)NH— or any combination thereof;
when A represents CH₂ and one of R₆ and R₇ represents R₉, where R represents unsubstituted nitrogen-containing monocyclic heterocyclyl, R₅ represents halogen, and the other one of R₆ and R₇ and/or R₈ each independently represent halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl;
when A represents CH₂ and one of R₅ and R₈ represents R₉, where R represents substituted nitrogen-containing monocyclic heterocyclyl, and the other one of R₅ and R₈, as well as R₆ and R₇ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, one to three of the other one of R₅ and R₈ as well as R₆ and R₇ represent halogen, and the nitrogen-containing monocyclic heterocyclyl is substituted with one or more substituents selected from the group consisting of hydroxy, amino, mercapto, nitro, halogen, CHO, cyano, C₁₋₆ alkyl, halogenated C₁₋₆ alkyl, C₃₋₆ cycloalkyl, C₁₋₆ alkoxy, C₁₋₆ alkyl-NH—, NH₂—C₁₋₆ alkylene, C₁₋₆ alkyl-NHC(O)—, C₁₋₆ alkyl-C(O)NH— or any combination thereof; or
when A represents CH₂ and one of R₅ and R₈ represents R₉, where R represents unsubstituted nitrogen-containing monocyclic heterocyclyl, and the other one of R₅ and R₈, as well as R₆ and R₇ each independently represent hydrogen, halogen, hydroxy, mercapto, nitro, amino, cyano, C₁₋₆ alkyl, C₁₋₆ alkoxy, halogenated C₁₋₆ alkyl, C₂₋₆ alkenyl, or C₂₋₆ alkynyl, the other one of R₅ and R₈, as well as R₆ and R₇ are not simultaneously hydrogen, and when R₆ is halogen, the other one of R₅ and R₈, as well as R₇ are not simultaneously hydrogen.

45-55. (canceled)

56. A method for treating or preventing a cereblon protein-mediated disease or disorder in a subject, comprising administering to the subject a therapeutically effective amount of the compound of Formula (I) or salts, enantiomers, stereoisomers, solvates, isotopically enriched analogs, prodrugs, or polymorphs thereof as claimed in claim 1, or a pharmaceutical composition comprising the compound of Formula (I) or a pharmaceutically acceptable salt thereof.

57. The method as claimed in claim 56, wherein the cereblon protein-mediated disease or disorder comprises: tumor, infectious disease, autoinflammatory disease, inflammatory disease, autoimmune disease, neurological disease, respiratory disease, anemia, hemorrhagic shock, transplant rejection, multiple organ dysfunction syndrome (MODS), sarcoidosis, adult respiratory distress syndrome, congestive heart failure, myocardial infarction, Unverricht's syndrome, Richter syndrome (RS), acute liver failure, and diabetes.

58. The method as claimed in claim 57, wherein the cereblon protein-mediated disease or disorder is selected from the group consisting of:
myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including acute myeloid leukemia, chronic myelogenous leukemia, B-cell chronic lymphocytic leukemia, leukemia-associated anemia, acute myeloid leukemia (AML); lymphoma, including diffuse large B-cell lymphoma, non-Hodgkin's lymphoma, anaplastic lymphoma, anaplastic large cell lymphoma, CD20 positive lymphoma, mantle cell lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; thyroid cancer; melanoma; lung cancer; inflammatory myofibroblastoma; colorectal cancer; brain glioma; astroblastoma; ovarian cancer; bronchial cancer; prostate cancer; breast cancer, including triple negative breast cancer, sporadic breast cancer and patients with Cowden syndrome; pancreatic cancer; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Unverricht's syndrome; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; autoinflammatory diseases, including gout, etc; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc.; septic shock; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; congestive heart failure; myocardial infarction; multiple organ dysfunction caused by cachexia and septic shock; and acute liver failure.

59. The method as claimed in claim 56, wherein the compound of Formula (I) or a pharmaceutically acceptable salt thereof, or the pharmaceutical composition is administered to the subject through at least one mode of administration selected from the group consisting of nasal administration, inhalation administration, topical administration, oral administration, oral mucosal administration, rectal administration, pleural cavity administration, peritoneal administration, vaginal administration, intramuscular administration, subcutaneous, transdermal, epidural, intrathecal, and intravenous administration.

60. A method for treating or preventing a disease or disorder in a subject, comprising administering to the subject a therapeutically effective amount of the compound of Formula (III) or salts, enantiomers, stereoisomers, solvates, isotopically enriched analogs, prodrugs, or polymorphs thereof as claimed in claim 20, or a pharmaceutical composition comprising the compound of Formula (III) or a pharmaceutically acceptable salt thereof, wherein the disease or disorder comprises tumor, infectious disease, autoinflammatory disease, inflammatory disease, autoimmune disease, neurological disease, respiratory disease, hemorrhagic shock, transplant rejection, multiple organ dysfunction syndrome (MODS), sarcoidosis, adult respiratory distress syndrome, congestive heart failure, myocardial infarction, Unverricht's syndrome, Richter syndrome (RS), acute liver failure, diabetes, Kennedy disease, seborrheic alopecia, hirsutism, skin disease, cardiovascular disease, dysfunctional uterine bleeding, anemia, pediatric aplastic anemia, endometriosis, transplant rejection, polycystic ovary syndrome, and thyroid disease.

61. The method as claimed in claim 60, wherein the disease or disorder is selected from the group consisting of:
myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including acute myeloid leukemia, chronic myelogenous leukemia, B-cell chronic lymphocytic leukemia, leukemia-associated anemia, acute myeloid leukemia (AML); lymphoma, including diffuse large B-cell lymphoma, non-Hodgkin's lymphoma, anaplastic lymphoma, anaplastic large cell lymphoma, CD20 positive lymphoma, mantle cell lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; thyroid cancer; melanoma; lung cancer, including lung adenocarcinoma, lung squamous cell carcinoma; inflammatory myofibroblastoma; colorectal cancer; brain glioma; astroblastoma; ovarian cancer; bronchial cancer; prostate cancer; breast cancer, including triple negative breast cancer, sporadic breast cancer and patients with Cowden syndrome; pancreatic cancer; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Unverricht's syndrome; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; autoinflammatory diseases, including gout, etc.; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc; septic shock; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; multiple organ dysfunction caused by cachexia and septic shock; acute liver failure; dysfunctional uterine bleeding; anemia; pediatric aplastic anemia; endometriosis; polycystic ovary syndrome; thyroid disease; cardiovascular diseases (e.g., coronary heart disease, congestive heart failure, myocardial infarction, atherosclerosis); skin diseases (e.g., acne); Kennedy disease; seborrheic alopecia; and hirsutism.

62. The method as claimed in claim 60, wherein the compound of Formula (III) or a pharmaceutically acceptable salt thereof, or the pharmaceutical composition is administered to the subject through at least one mode of administration selected from the group consisting of nasal administration, inhalation administration, topical administration, oral administration, oral mucosal administration, rectal administration, pleural cavity administration, peritoneal administration, vaginal administration, intramuscular administration, subcutaneous, transdermal, epidural, intrathecal, and intravenous administration.

63. A pharmaceutical composition comprising as active ingredient the compound of Formula (II) or pharmaceutically acceptable salts, enantiomers, stereoisomers, solvates, isotopically enriched analogs, prodrugs, or polymorphs thereof as claimed in claim 9, and at least one pharmaceutically acceptable carrier;
optionally comprising at least one additional therapeutic agent.

64. A medicine kit or reagent kit comprising:
the compound of Formula (II) or a pharmaceutically acceptable salt thereof as claimed in claim 9, or a pharmaceutical composition comprising the compound of Formula (II) or a pharmaceutically acceptable salt thereof.

65. A medicine kit or reagent kit comprising:
the compound of Formula (III) or a pharmaceutically acceptable salt thereof as claimed in claim 20, or a pharmaceutical composition comprising the compound of Formula (III) or a pharmaceutically acceptable salt thereof.

66. A method for treating or preventing a cereblon protein-mediated disease or disorder in a subject, comprising administering to the subject a therapeutically effective amount of the compound of Formula (II) or salts, enantiomers, stereoisomers, solvates, isotopically enriched analogs, prodrugs, or polymorphs thereof as claimed in claim 9, or a pharmaceutical composition comprising the compound of Formula (II) or a pharmaceutically acceptable salt thereof.

67. The method as claimed in claim 66, wherein the cereblon protein-mediated disease or disorder comprises: tumor, infectious disease, autoinflammatory disease, inflammatory disease, autoimmune disease, neurological disease, respiratory disease, anemia, hemorrhagic shock, transplant rejection, multiple organ dysfunction syndrome (MODS), sarcoidosis, adult respiratory distress syndrome, congestive heart failure, myocardial infarction, Unverricht's syndrome, Richter syndrome (RS), acute liver failure, and diabetes.

68. The method as claimed in claim 66, wherein the cereblon protein-mediated disease or disorder is selected from the group consisting of:
myeloma, including multiple myeloma, plasma cell myeloma, smoldering myeloma, smoldering multiple myeloma; myelofibrosis; bone marrow disease; myelodysplastic syndrome (MDS); previously treated myelodysplastic syndrome; transplantation-related cancer; neutropenia; leukemia, including acute myeloid leukemia, chronic myelogenous leukemia, B-cell chronic lymphocytic leukemia, leukemia-associated anemia, acute myeloid leukemia (AML); lymphoma, including diffuse large B-cell lymphoma, non-Hodgkin's lymphoma, anaplastic lymphoma, anaplastic large cell lymphoma, CD20 positive lymphoma, mantle cell lymphoma, primary lymphoma, B-cell lymphoma, recurrent B-cell non-Hodgkin's lymphoma, recurrent diffuse large B-cell lymphoma, recurrent mediastinal (thymic) large B-cell lymphoma, primary mediastinal (thymic) large B-cell lymphoma, recurrent transformed non-Hodgkin's lymphoma, refractory B-cell non-Hodgkin's lymphoma, refractory diffuse large B-cell lymphoma, refractory primary mediastinal (thymic) large B-cell lymphoma, refractory transformed non-Hodgkin's lymphoma; thyroid cancer; melanoma; lung cancer; inflammatory myofibroblastoma; colorectal cancer; brain glioma; astroblastoma; ovarian cancer; bronchial cancer; prostate cancer; breast cancer, including triple negative breast cancer, sporadic breast cancer and patients with Cowden syndrome; pancreatic cancer; neuroblastoma; extramedullary plasmacytoma; plasmacytoma; gastric cancer; gastrointestinal stromal tumors; esophageal cancer; colorectal adenocarcinoma; esophageal squamous cell carcinoma; liver cancer; renal cell carcinoma; bladder cancer; endometrial cancer; brain cancer; oral cancer; sarcoma, including rhabdomyosarcoma, various lipogenic tumors, Ewing's sarcoma/primitive neuroectodermal tumors (Ewing/PNETs), and leiomyosarcoma; urothelial carcinoma; basal cell carcinoma; oral squamous cell carcinoma; cholangiocarcinoma; bone cancer; cervical cancer; skin cancer; Unverricht's syndrome; Richter syndrome (RS); sepsis syndrome; autoimmune diseases, including rheumatoid arthritis, autoimmune encephalomyelitis, ankylosing spondylitis, psoriasis, systemic lupus erythematosus, multiple sclerosis, recurrent oral ulcers, Kawasaki disease, polymyositis/dermatomyositis, Sjogren's syndrome, and atopic dermatitis; keratoconjunctivitis; autoinflammatory diseases, including gout, etc; inflammatory diseases, including Crohn's disease and ulcerative colitis, pneumonia, osteoarthritis, synovitis, systemic inflammatory response syndrome, airway inflammation, bronchitis; cerebral malaria; infectious diseases, including viral pneumonia, Acquired immunodeficiency syndrome (AIDS), COVID-19 novel coronavirus infection, gram-negative bacteria infection, gram-positive bacteria infection, tuberculosis, etc.; septic shock; bacterial meningitis; chronic obstructive pulmonary disease; asthma; hemorrhagic shock; organ (including kidney, heart, lung) or tissue transplantation rejection; diabetes; sarcoidosis; adult respiratory distress syndrome; congestive heart failure; myocardial infarction; multiple organ dysfunction caused by cachexia and septic shock; and acute liver failure.

69. The method as claimed in claim 66, wherein the compound of Formula (II) or a pharmaceutically acceptable salt thereof, or the pharmaceutical composition is administered to the subject through at least one mode of administration selected from the group consisting of nasal administration, inhalation administration, topical administration, oral administration, oral mucosal administration, rectal administration, pleural cavity administration, peritoneal administration, vaginal administration, intramuscular administration, subcutaneous, transdermal, epidural, intrathecal, and intravenous administration.

70. A method, comprising preparing of the compound of Formula (III) as claimed in claim 20 from a compound of Formula (II): 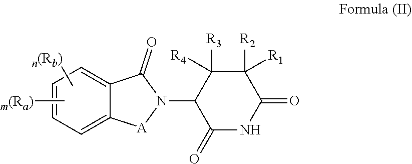
or salts, enantiomers, stereoisomers, solvates, isotopically enriched analogs, prodrugs, or polymorphs thereof,
wherein A represents CO, CH₂ or CD₂;
R₁, R₂, R₃ and R₄ are the same or different and each independently represent hydrogen, deuterium, halogen, optionally deuterated C₁₋₆ alkyl, optionally deuterated C₁₋₆ alkoxy, or halogenated C₁₋₆ alkyl;
(R_{b})ₙ indicates that benzene ring is substituted with n R_{b} substituents, and each R_{b} are the same or different and each independently represent hydrogen, deuterium, halogen, hydroxy, mercapto, nitro, amino, cyano, optionally deuterated C₁₋₆ alkyl, optionally deuterated C₁₋₆ alkoxy, halogenated C₁₋₆ alkyl, C₂₋₆ alkenyl or C₂₋₆ alkynyl;
(Rₐ)ₘ indicates that the benzene ring is substituted with m Rₐ substituents, and each Rₐ are the same or different and each independently represents the structure of the following formula:
W-LIN—U₂—R_{c}—U₁—,
wherein
U₁ and U₂ are the same or different and each independently represent a bond, N(R_{d}) or O, wherein R_{d} represents H or optionally deuterated C₁₋₆ alkyl;
R_{c} represents optionally substituted nitrogen-containing monocyclic heterocyclylene or optionally substituted nitrogen-containing bridged heterocyclylene;
W represents hydrogen, optionally substituted nitrogen-containing monocyclic heterocyclyl, optionally substituted nitrogen-containing bridged heterocyclyl, optionally substituted cycloalkyl, optionally substituted heteroaryl, optionally substituted aryl, C₂₋₆ alkenyl, C₂₋₆ alkynyl, a leaving group, CHO, COOH, NH₂, hydroxy or mercapto; and
wherein in case that the compound of Formula (II) is partially or fully deuterated:
when W represents optionally substituted nitrogen-containing monocyclic heterocyclyl, optionally substituted nitrogen-containing bridged heterocyclyl, optionally substituted cycloalkyl, optionally substituted heteroaryl or optionally substituted aryl, LIN represents a bond, optionally substituted methylene, or optionally substituted linear or branched C₂₋₃₀ alkylene, wherein one or more groups Rₑ and/or one or more groups R_{f} and/or any combination of one or more groups Rₑ and R_{f} are optionally inserted into the backbone carbon chain of the linear or branched C₂₋₃₀ alkylene group, wherein carbon-carbon bond between one or more pairs of adjacent carbon atoms in the backbone carbon chain is interrupted by the group Rₑ, R_{f}, or a combination of Rₑ and R_{f}; wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl, and in case that two or more groups Rₑ are inserted into the backbone carbon chain of the linear or branched C₂₋₃₀ alkylene group, the two or more groups Rₑ are not directly connected to each other; and wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano or any combination thereof; or
when W represents hydrogen, C₂₋₆ alkenyl, C₂₋₆ alkynyl, a leaving group, CHO, COOH, NH₂, hydroxy or mercapto, LIN represents optionally substituted methylene, or optionally substituted linear or branched C₂₋₃₀ alkylene, wherein one or more groups Rₑ and/or one or more groups R_{f} and/or any combination of one or more groups Rₑ and R_{f} are optionally inserted into the backbone carbon chain of the linear or branched C₂₋₃₀ alkylene group, wherein carbon-carbon bond between one or more pairs of adjacent carbon atoms in the backbone carbon chain is interrupted by the group Rₑ, R_{f}, or a combination of Rₑ and R_{f}; wherein each Rₑ are selected from the group consisting of O, N(R_{g}), C(O), C(O)O, OC(O), S(O), S(O)₂, S(O)₂NH, NHS(O)₂, C(O)N(R_{g}), N(R_{g})C(O), or N(R_{g})C(O)N(R_{g}), where each R_{g} independently represent H or C₁₋₆ alkyl, and in case that two or more groups Rₑ are inserted into the backbone carbon chain of the linear or branched C₂₋₃₀ alkylene group, the two or more groups Rₑ are not directly connected to each other; and wherein each R_{f} are selected from the group consisting of cycloalkylene, arylene, heterocyclylene, heteroarylene, alkynylene, alkenylene, or any combination thereof, wherein the cycloalkylene, arylene, heterocyclylene, and heteroarylene are each independently optionally substituted with a substituent selected from the group consisting of deuterium, optionally deuterated C₁₋₆ alkyl, optionally deuterated C₃₋₆ cycloalkyl, hydroxy, amino, mercapto, halogen, optionally deuterated C₁₋₆ alkoxy, optionally deuterated C₁₋₆ alkyl-NH—, halogenated C₁₋₆ alkyl, NH₂—C₁₋₆ alkylene, optionally deuterated C₁₋₆ alkyl-NHC(O)—, optionally deuterated C₁₋₆ alkyl-C(O)NH—, cyano or any combination thereof; or
in case that the compound of Formula (II) is not deuterated, LIN represents optionally substituted methylene, and W represents optionally substituted nitrogen-containing monocyclic heterocyclyl or optionally substituted nitrogen-containing bridged heterocyclyl; and
m represents an integer of 1, 2, 3 or 4, n represents an integer of 0, 1, 2 or 3, and the sum of m and n is an integer of 1, 2, 3 or 4;
with the proviso that when the compound of Formula (II) is not deuterated, the following compounds are excluded:
3-(4,6-difluoro-1-oxo-5-(4-(piperidin-4-ylmethyl)piperazin-1-yl)isoindolin-2-yl)piperidine-2,6-dione;
2-(2,6-dioxopiperidin-3-yl)-5-(4-(piperidin-4-ylmethyl)piperazin-1-yl)isoindoline-1,3-dione;
3-(4-chloro-1-oxo-5-(4-(piperidin-4-ylmethyl)piperidin-1-yl)isoindolin-2-yl)piperidine-2,6-dione;
5-(4-(azetidin-3-ylmethyl)piperazin-1-yl)-2-(2,6-dioxopiperidin-3-yl)isoindoline-1,3-dione; and
2-(2,6-dioxopiperidin-3-yl)-5-(3-(piperazin-1-ylmethyl)azetidin-1-yl)isoindoline-1,3-dione.
